(19) United States
(12) Patent Application Publication
(10) Pub. No.: US 2023/0086039 A1
(43) Pub. Date: Mar. 23, 2023

(54) ORGANIC LIGHT EMITTING DEVICE

(71) Applicant: LG CHEM, LTD., Seoul (KR)

(72) Inventors: Minjun KIM, Daejeon (KR); Dong Hoon LEE, Daejeon (KR); Yongbum CHA, Daejeon (KR); Sang Duk SUH, Daejeon (KR); Young Seok KIM, Daejeon (KR); Joongsuk OH, Daejeon (KR); Seoyeon KIM, Daejeon (KR)

(21) Appl. No.: 17/617,111

(22) PCT Filed: Mar. 11, 2021
(86) PCT No.: PCT/KR2021/003037

§ 371 Date: Dec. 7, 2021

(30) Foreign Application Priority Data
Mar. 11, 2020 (KR) 10-2020-0030232
Mar. 11, 2021 (KR) 10-2021-0031954

(51) Int. Cl. H01L 51/00; C07D 405/04; C09K 11/06; C07D 409/14; C07D 405/14; C07D 405/10; C07C 211/54; C07D 307/91; C07D 333/76; C07C 211/61; C07D 409/12

(52) U.S. Cl. CPC H01L 51/0073; C07D 405/04; C09K 11/06; H01L 51/0067; H01L 51/0052; H01L 51/0054; C07D 409/14; H01L 51/0074; H01L 51/0058; C07D 405/14; C07D 405/10; C07C 211/54; H01L 51/006; C07D 307/91; H01L 51/0061; C07D 333/76; C07C 211/61; C07D 409/12; H01L 51/5012

(57) ABSTRACT

Provided is an organic light emitting device comprising a light emitting layer comprising a compound of Chemical Formula 1 and a compound of Chemical Formula 2:

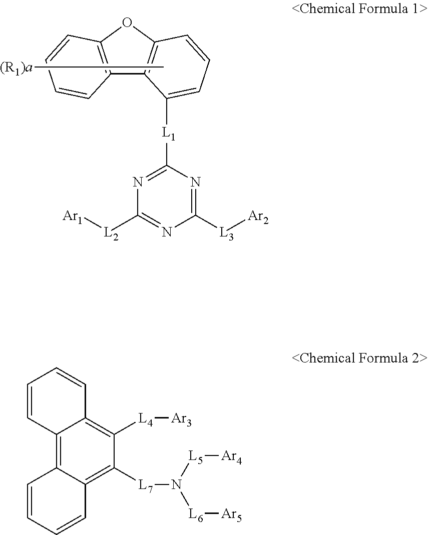

wherein:
Ar₁, Ar₂, Ar₄ and Ar₅ are each independently a substituted or unsubstituted C₆₋₆₀ aryl or C₂₋₆₀ heteroaryl containing N, O or S;
Ar₃ is hydrogen, or a substituted or unsubstituted C₆₋₆₀ aryl or C₂₋₆₀ heteroaryl containing N, O or S;
L₁ to L₃ are each independently a single bond or a substituted or unsubstituted C₆₋₆₀ arylene;
L₄ to L₆ are each independently a single bond or a substituted or unsubstituted C₆₋₆₀ arylene or C₂₋₆₀ heteroarylene containing N, O or S;
L₇ is a substituted or unsubstituted C₆₋₆₀ arylene;
R₁ is hydrogen, deuterium, or a substituted or unsubstituted C₆₋₆₀ aryl or C₂₋₆₀ heteroaryl containing one or of N, O and S;
a is 0 to 7.

### CROSS-REFERENCE TO RELATED APPLICATIONS

This application is a National Stage Application of International Application No. PCT/KR2021/003037 filed on Mar. 11, 2021, which claims priority to and the benefit of Korean Patent Application No. 10-2020-0030232 filed in the Korean Intellectual Property Office on Mar. 11, 2020, and Korean Patent Application No. 10-2021-0031954 filed in the Korean Intellectual Property Office on Mar. 11, 2021, the entire contents of which are incorporated herein by reference.

### FIELD OF THE INVENTION

The present disclosure relates to relates to an organic light emitting device having improved driving voltage, efficiency and lifetime.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer disposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

In the organic light emitting devices as described above, there is a continuing need for the development of an organic light emitting device having improved driving voltage, efficiency and lifetime.

### PRIOR ART LITERATURE

### Patent Literature

(Patent Literature 1) Korean Unexamined Patent Publication No. 10-2000-0051826

### SUMMARY OF THE INVENTION

### Technical Problem

The present disclosure relates to an organic light emitting device having improved driving voltage, efficiency and lifetime.

### Technical Solution

The following organic light emitting device is provided herein:

An organic light emitting device including: an anode, a cathode, and a light emitting layer disposed between the anode and the cathode,

wherein the light emitting layer includes a compound of the following Chemical Formula 1 and a compound of the following Chemical Formula 2:

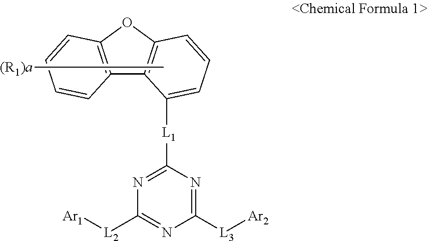

wherein in Chemical Formula 1:

Ar₁ and Ar₂ are each independently a substituted or unsubstituted C₆₋₆₀ aryl or a substituted or unsubstituted C₂₋₆₀ heteroaryl containing one or more selected from the group consisting of N, O and S;

L₁ to L₃ are each independently a single bond or a substituted or unsubstituted C₆₋₆₀ arylene;

R₁ is hydrogen, deuterium, a substituted or unsubstituted C₆₋₆₀ aryl, or a substituted or unsubstituted C₂₋₆₀ heteroaryl containing one or more selected from the group consisting of N, O and S;

a is an integer of 0 to 7;

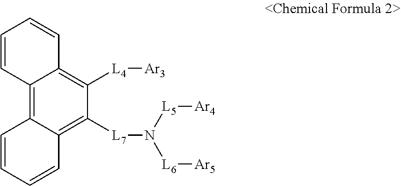

wherein in Chemical Formula 2:

Ar₃ is hydrogen, a substituted or unsubstituted C₆₋₆₀ aryl or a substituted or unsubstituted C₂₋₆₀ heteroaryl containing one or more selected from the group consisting of N, O and S;

Ar₄ and Ar₅ are each independently a substituted or unsubstituted C₆₋₆₀ aryl or a substituted or unsubstituted C₂₋₆₀ heteroaryl containing one or more selected from the group consisting of N, O and S;

L₄ to L₆ are each independently a single bond, a substituted or unsubstituted C₆₋₆₀ arylene, or a substituted or unsubstituted C₂₋₆₀ heteroarylene containing one or more selected from the group consisting of N, O and S; and

L₇ is a substituted or unsubstituted C₆₋₆₀ arylene.

### Advantageous Effects

The above-mentioned organic light emitting device can improve the efficiency, achieve low driving voltage and/or improve lifetime characteristics by containing the compound of Chemical Formula 1 and the compound of Chemical Formula 2 in the light emitting layer.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION

Hereinafter, embodiments of the present disclosure will be described in more detail to facilitate understanding of the invention.

As used herein, the notation [symbol] or [symbol] means a bond linked to another substituent group.

As used herein, the term “substituted or unsubstituted” means being unsubstituted or substituted with one or more substituents selected from the group consisting of deuterium, a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, or a heteroaryl containing at least one of N, O and S atoms, or being unsubstituted or substituted with a substituent to which two or more substituents of the above-exemplified substituents are connected. For example, “a substituent in which two or more substituents are connected” can be a biphenyl group. Namely, a biphenyl group can be an aryl group, or it can also be interpreted as a substituent in which two phenyl groups are connected.

In the present disclosure, the carbon number of a carbonyl group is not particularly limited, but is preferably 1 to 40. Specifically, the carbonyl group can be a compound having the following structural formulas, but is not limited thereto:

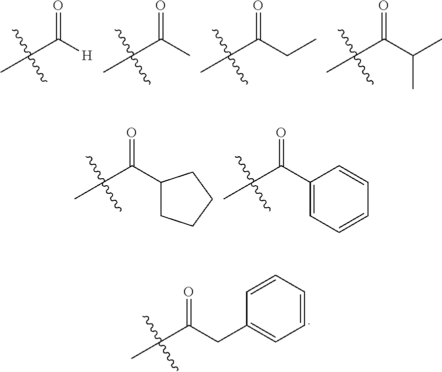

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a compound having the following structural formulas, but is not limited thereto:

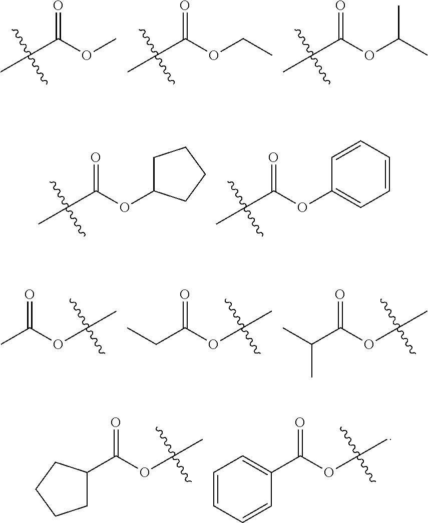

In the present disclosure, the carbon number of an imide group is not particularly limited, but is preferably 1 to 25. Specifically, the imide group can be a compound having the following structural formulas, but is not limited thereto:

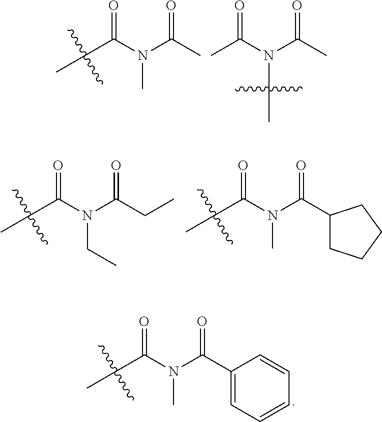

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, or the like, but is not limited thereto.

In the present disclosure, the fluorenyl group can be substituted, and two substituents can be linked with each other to form a spiro structure. In the case where the fluorenyl group is substituted,

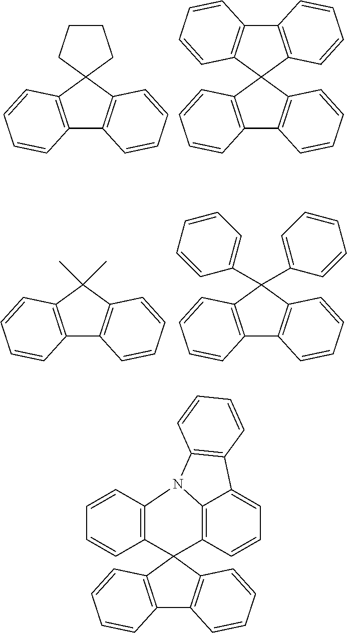

and the like can be formed. However, the structure is not limited thereto.

In the present disclosure, a heterocyclic group is a heterocyclic group containing one or more of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can be applied to the aforementioned description of the heterocyclic group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heteroaryl group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocyclic group is not a monovalent group but formed by combining two substituent groups.

Hereinafter, the present disclosure will be described in detail for each configuration.

### Anode and Cathode

The anode and cathode used in the present disclosure mean electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SNO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

### Hole Injection Layer

The organic light emitting device according to the present disclosure can further include a hole injection layer on the anode, if necessary.

The hole injection layer is a layer injecting holes from an electrode, and the hole injection material is preferably a compound which has an ability of transporting the holes, a hole injection effect in the anode and an excellent hole injection effect to the light emitting layer or the light emitting material, prevents movement of an exciton generated in the light emitting layer to the electron injection layer or the electron injection material, and has an excellent thin film forming ability. It is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaaza-triphenylene-based organic material, a quinacridone-based organic material, a perylene-based organic material, anthraquinone, polyaniline and polythiophene-based conductive polymer, and the like, but are not limited thereto.

### Hole Transport Layer

The organic light emitting device according to the present disclosure can include a hole transport layer on the anode (or on a hole injection layer when the hole injection layer is present), if necessary.

The hole transport layer is a layer that receives holes from an anode or a hole injection layer and transports the holes to the light emitting layer. The hole transport material is suitably a material having large mobility to the holes, which can receive holes from the anode or the hole injection layer and transfer the holes to the light emitting layer.

Specific examples of the hole transport material include an arylamine-based organic material, a conductive polymer, a block copolymer in which a conjugate portion and a non-conjugate portion are present together, and the like, but are not limited thereto.

### Electron Blocking Layer

The electron blocking layer is a layer provided between the hole transport layer and the light emitting layer in order to prevent the electrons injected in the cathode from being transferred to the hole transport layer without being recombined in the light emitting layer, which can also be referred to as an electron inhibition layer or an electron stopping layer. The electron blocking layer is preferably a material having a smaller electron affinity than the electron transport layer.

### Light Emitting Layer

The light emitting layer used in the present disclosure means a layer that can emit light in the visible light region by combining holes and electrons transported from the anode and the cathode. Generally, the light emitting layer includes a host material and a dopant material, and in the present disclosure, the compound of Chemical Formula 1 and the compound of Chemical Formula 2 are included as a host.

Preferably, the compound of Chemical Formula 1 can be of any one of the following Chemical Formulas 1-1 to 1-3:

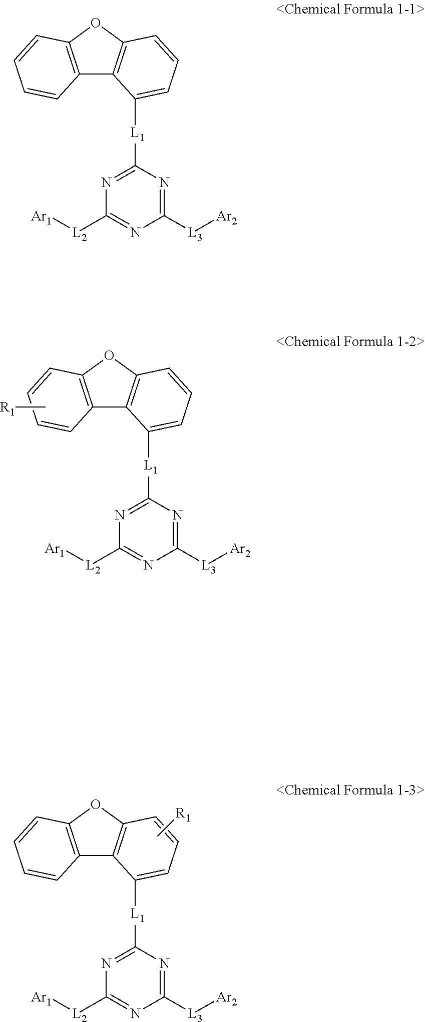

wherein in Chemical Formulas 1-1 to 1-3:

Ar₁ and Ar₂, L₁ to L₃ and R₁ are as defined in Chemical Formula 1.

Preferably, Ar₁ and Ar₂ can be each independently a substituted or unsubstituted C₆₋₂₀ aryl or a substituted or unsubstituted C₂₋₂₀ heteroaryl containing one or more selected from the group consisting of N, O and S,

more preferably, Ar₁ and Ar₂ can be each independently phenyl, biphenylyl, terphenylyl, naphthyl, phenanthrenyl, dibenzofuranyl, or dibenzothiophenyl, and

most preferably, Ar₁ and Ar₂ can be each independently any one selected from the group consisting of the following:

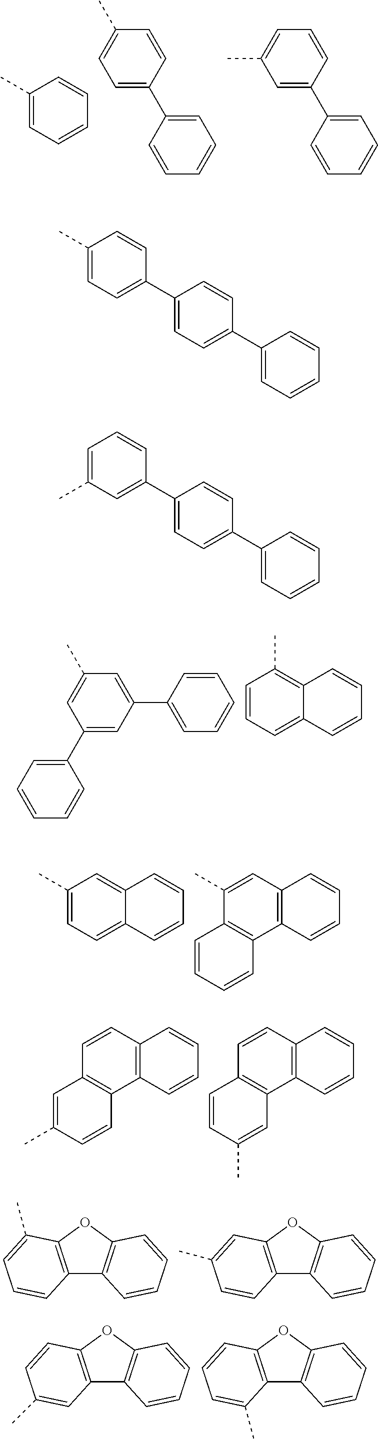

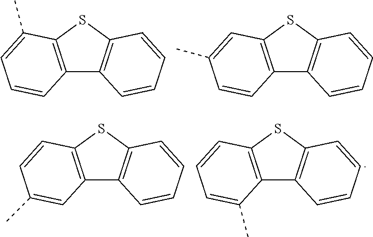

Preferably, L₁ to L₃ can be each independently a single bond or a substituted or unsubstituted C₆₋₂₀ arylene,

more preferably, L₁ to L₃ can be each independently a single bond, phenylene, biphenylylene or naphthylene, and

most preferably, L₁ to L₃ can be each independently a single bond or any one selected from the group consisting of the following:

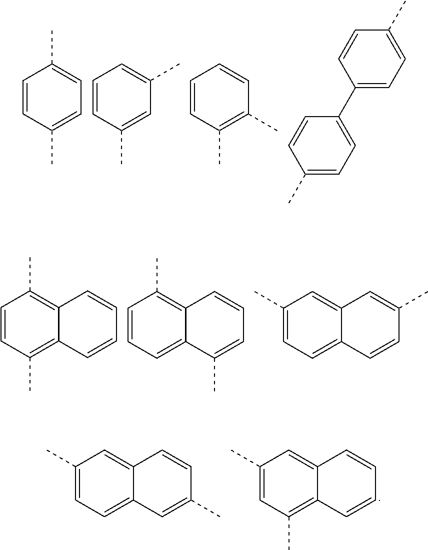

Preferably, each R₁ can be independently hydrogen, deuterium, a substituted or unsubstituted C₆₋₂₀ aryl, or a substituted or unsubstituted C₂₋₂₀ heteroaryl containing one or more selected from the group consisting of N, O and S, and

more preferably, each R₁ can be independently hydrogen, deuterium, phenyl, biphenylyl, terphenylyl, naphthyl, phenanthrenyl, triphenylenyl, naphthyl phenyl, phenyl naphthyl, fluoranthenyl, dihydroindenyl, dibenzofuranyl, dibenzothiophenyl, benzonaphthofuranyl, or benzonaphthothiophenyl.

Preferably, a can be 0 or 1. More preferably, a can be 1.

Preferably, at least one of Ar₁, Ar₂ and R₁ can be naphthyl, phenyl naphthyl, naphthyl phenyl, phenanthrenyl, fluoranthenyl, dibenzofuranyl, dibenzothiophenyl, benzonaphthofuranyl, or benzonaphthothiophenyl.

More preferably, at least one of Ar₁, Ar₂ and R₁ can be naphthyl, phenyl naphthyl, naphthyl phenyl, fluoranthenyl, dibenzofuranyl, benzonaphthofuranyl, or benzonaphthothiophenyl.

Representative examples of the compound of Chemical Formula 1 are as follows:

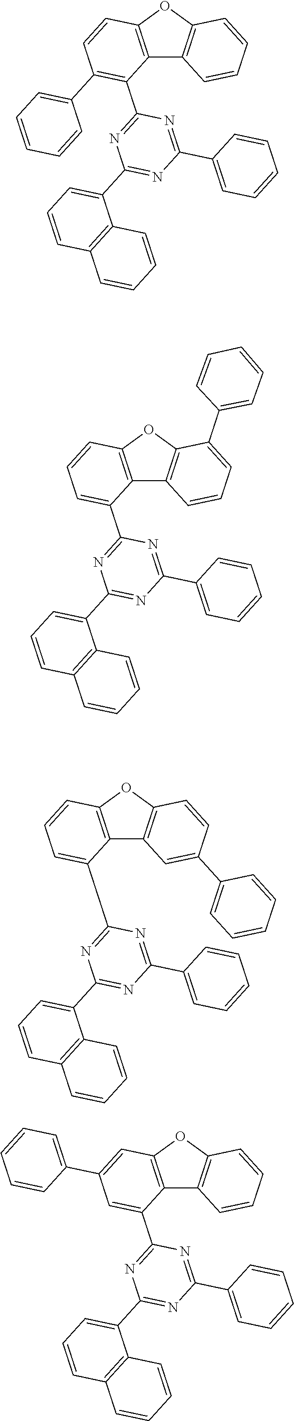

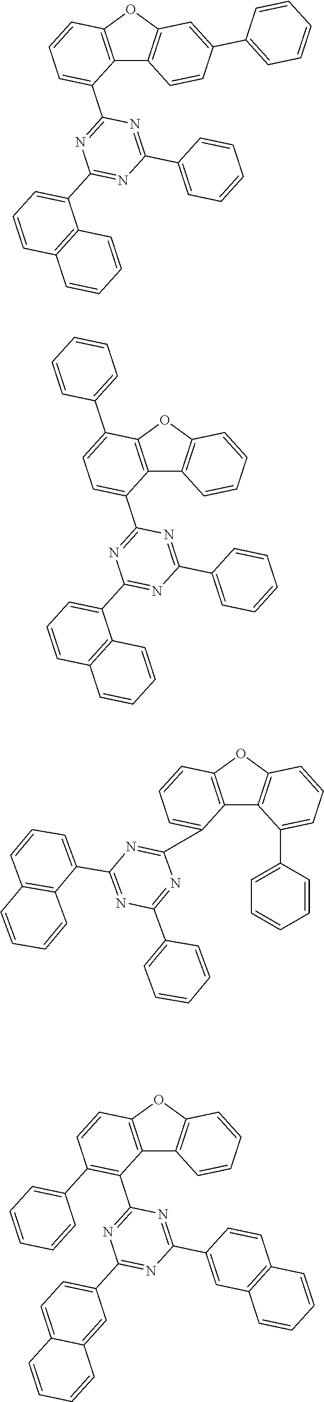 [SMILES: c1ccc(-c2ccc3c(c2)oc2cccc(-c4nc(-c5ccccc5)nc(-c5cccc6ccccc56)n4)c23)cc1 ; c1ccc(-c2nc(-c3cccc4ccccc34)nc(-c3ccc(-c4ccccc4)c4oc5ccccc5c34)n2)cc1 ; c1ccc(-c2nc(-c3cccc4ccccc34)nc(-c3cccc4oc5cccc(-c6ccccc6)c5c34)n2)cc1 ; c1ccc(-c2ccc3oc4ccccc4c3c2-c2nc(-c3ccc4ccccc4c3)nc(-c3ccc4ccccc4c3)n2)cc1]

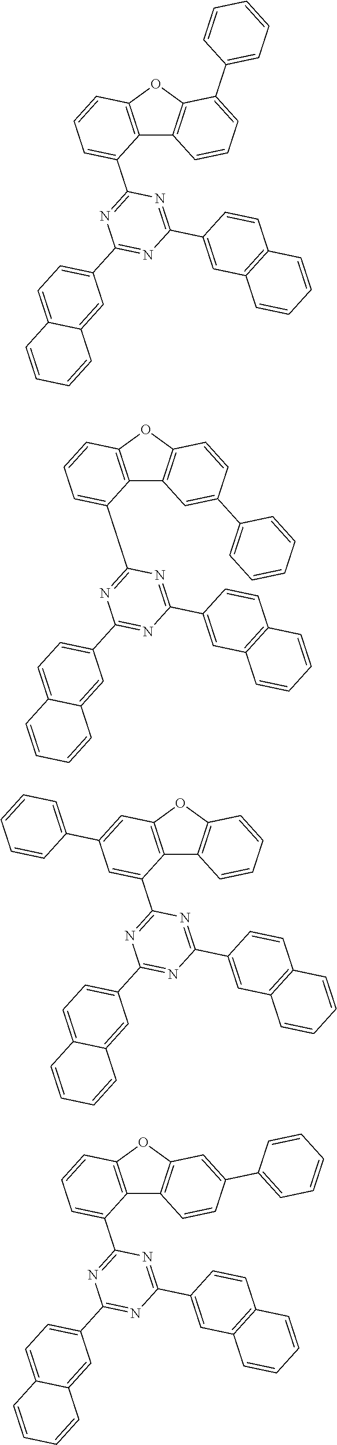

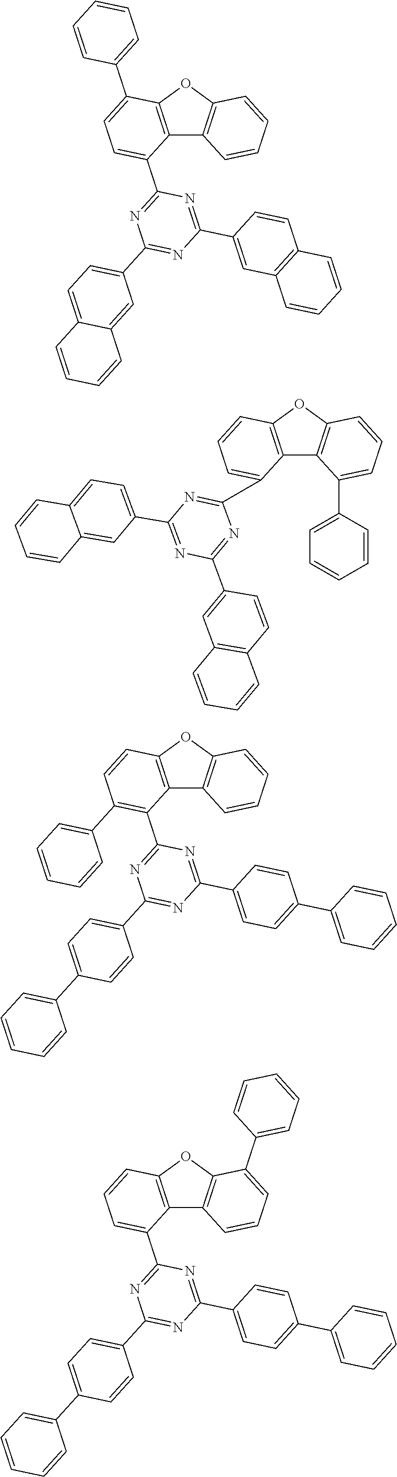

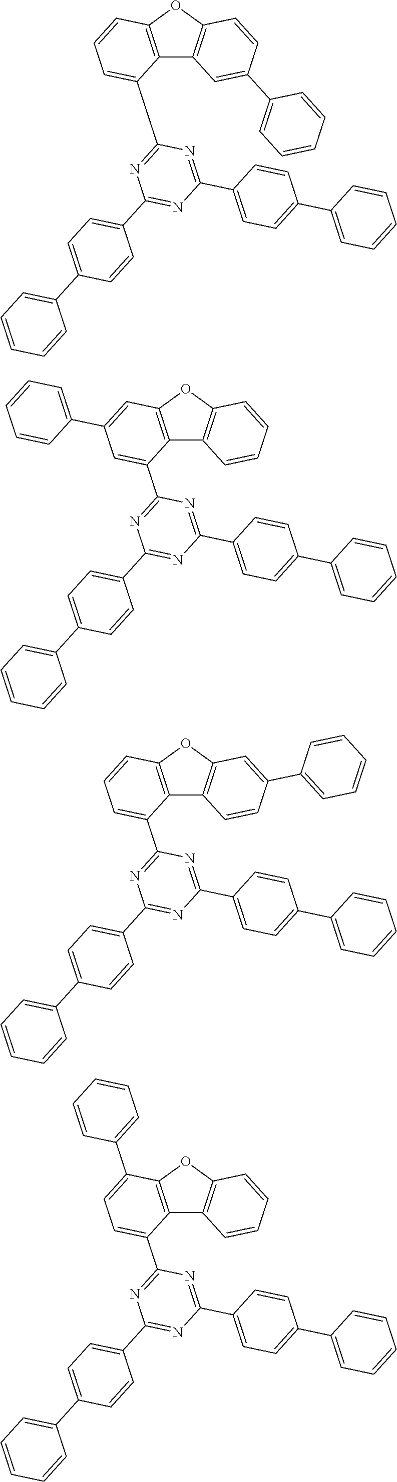

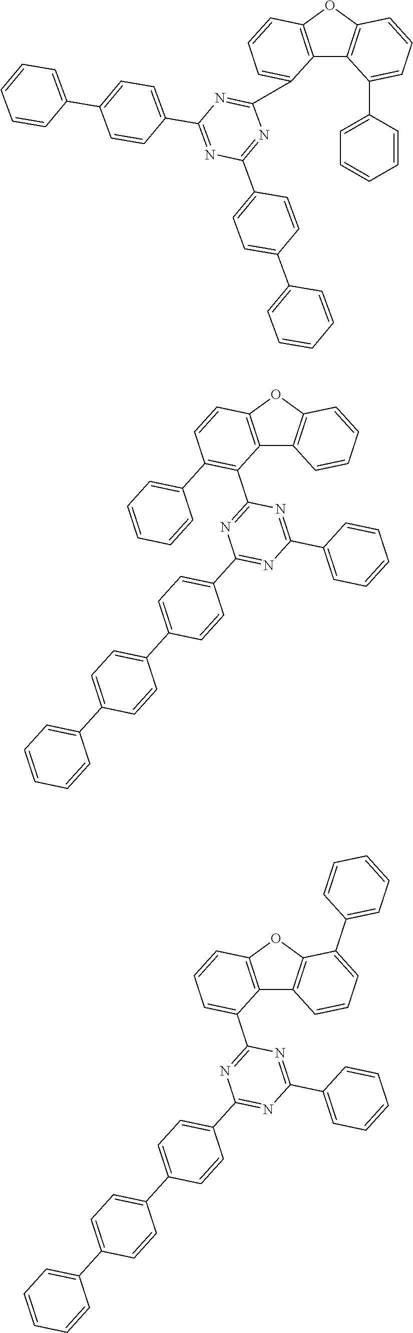 [SMILES: c1ccc(-c2ccc(-c3nc(-c4ccc(-c5ccccc5)cc4)nc(-c4cccc5oc6cccc(-c7ccccc7)c6c45)n3)cc2)cc1 ; c1ccc(-c2ccc(-c3ccc(-c4nc(-c5ccccc5)nc(-c5c(-c6ccccc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1]

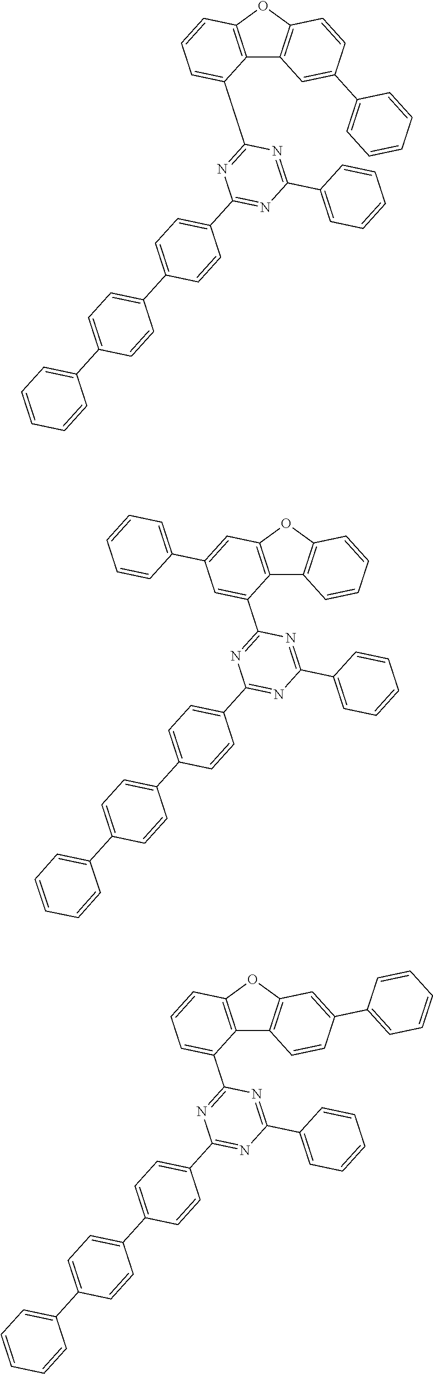 [SMILES: c1ccc(-c2ccc(-c3ccc(-c4nc(-c5ccccc5)nc(-c5cccc6oc7ccc(-c8ccccc8)cc7c56)n4)cc3)cc2)cc1 ; c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccccc8)ccc7c56)n4)cc3)cc2)cc1]

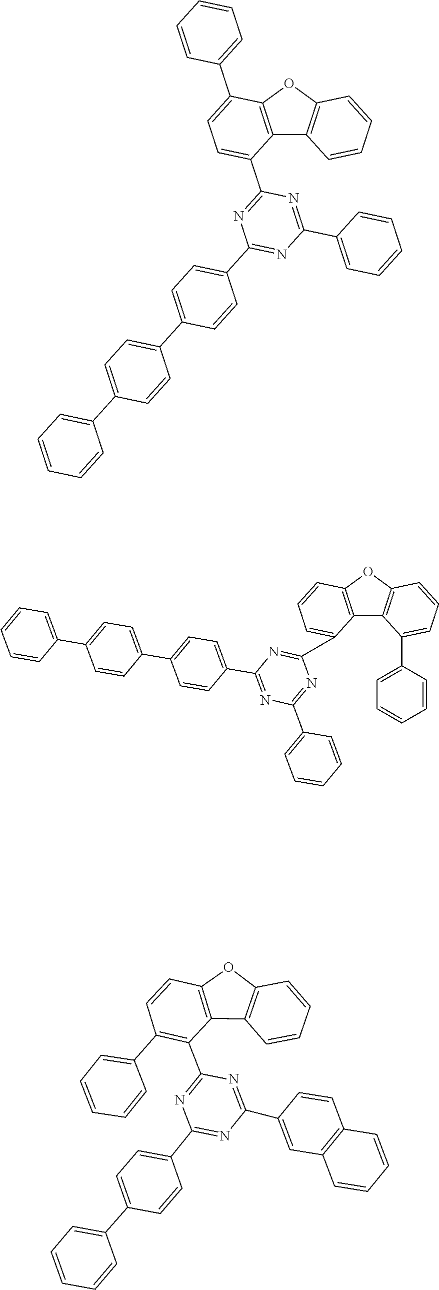 [SMILES: c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1 ; c1ccc(-c2ccc(-c3nc(-c4ccc5ccccc5c4)nc(-c4c(-c5ccccc5)ccc5oc6ccccc6c45)n3)cc2)cc1]

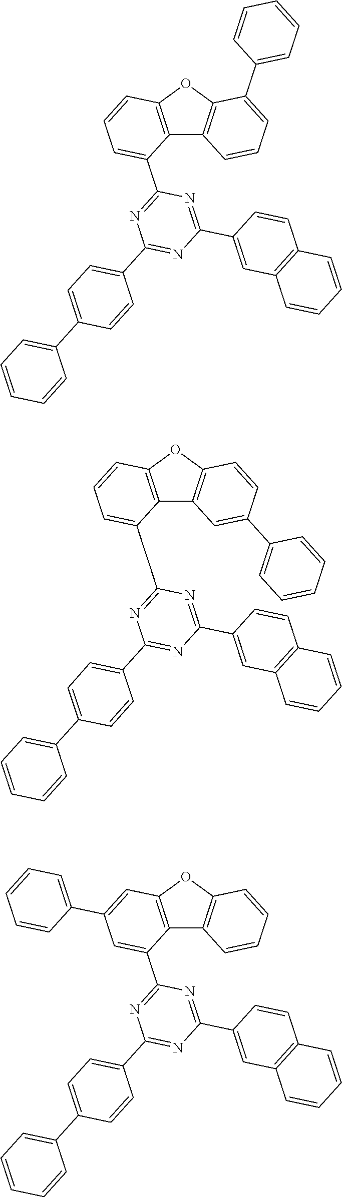 [SMILES: c1ccc(-c2ccc(-c3nc(-c4ccc5ccccc5c4)nc(-c4cccc5oc6c(-c7ccccc7)cccc6c45)n3)cc2)cc1 ; c1ccc(-c2ccc(-c3nc(-c4ccc5ccccc5c4)nc(-c4cccc5oc6ccc(-c7ccccc7)cc6c45)n3)cc2)cc1 ; c1ccc(-c2ccc(-c3nc(-c4ccc5ccccc5c4)nc(-c4cc(-c5ccccc5)cc5oc6ccccc6c45)n3)cc2)cc1]

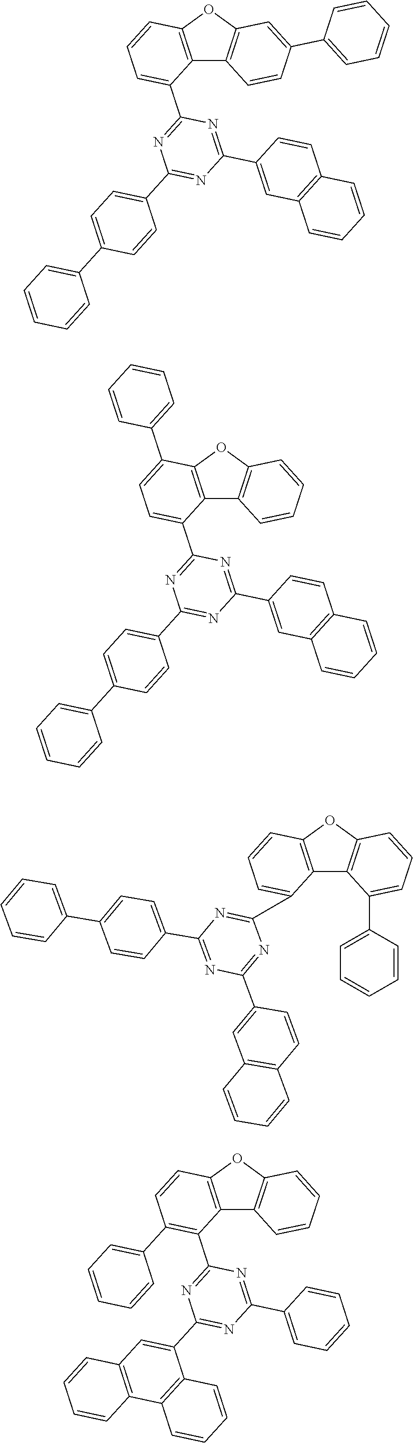

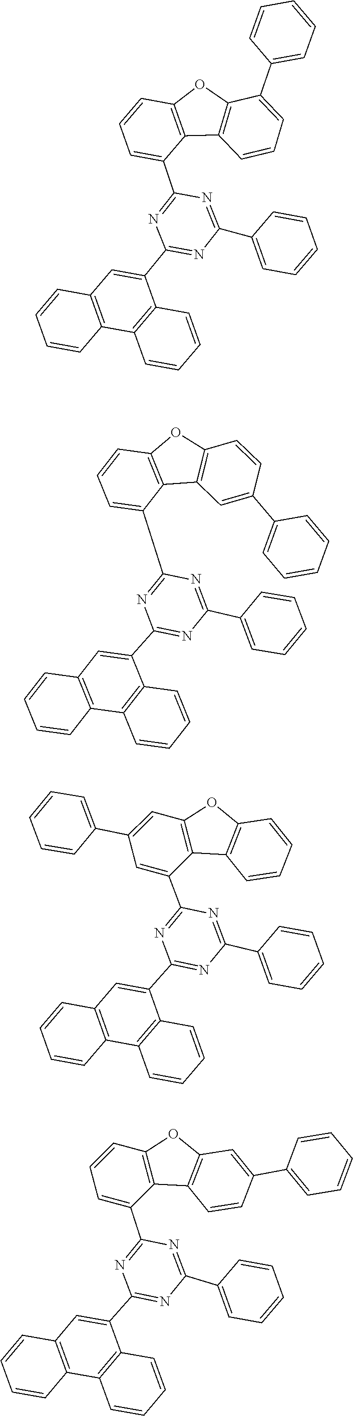 [SMILES: c1ccc(-c2nc(-c3cc4ccccc4c4ccccc34)nc(-c3cccc4oc5c(-c6ccccc6)cccc5c34)n2)cc1 ; c1ccc(-c2ccc3oc4cccc(-c5nc(-c6ccccc6)nc(-c6cc7ccccc7c7ccccc67)n5)c4c3c2)cc1 ; c1ccc(-c2cc(-c3nc(-c4ccccc4)nc(-c4cc5ccccc5c5ccccc45)n3)c3c(c2)oc2ccccc23)cc1 ; c1ccc(-c2ccc3c(c2)oc2cccc(-c4nc(-c5ccccc5)nc(-c5cc6ccccc6c6ccccc56)n4)c23)cc1]

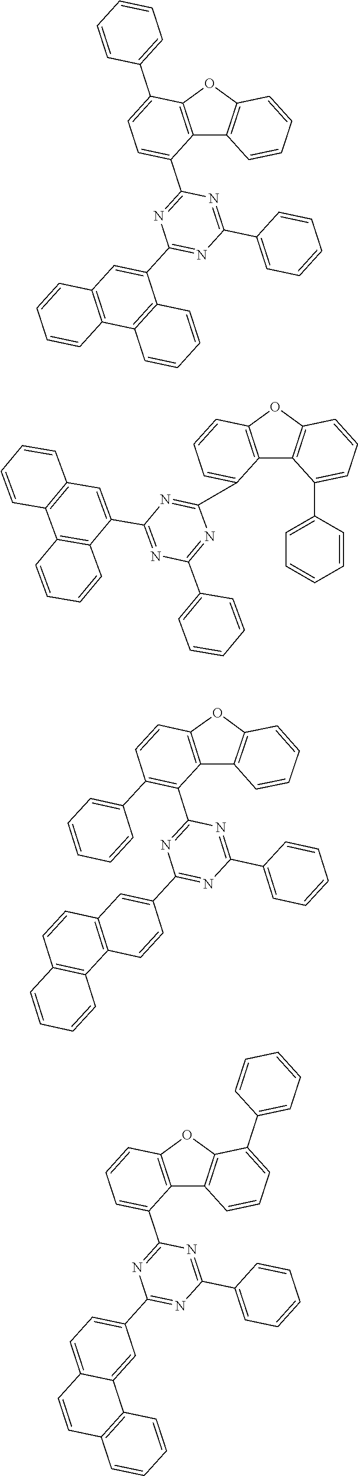 [SMILES: c1ccc(-c2nc(-c3cc4ccccc4c4ccccc34)nc(-c3ccc(-c4ccccc4)c4oc5ccccc5c34)n2)cc1 ; c1ccc(-c2nc(-c3cc4ccccc4c4ccccc34)nc(-c3cccc4oc5cccc(-c6ccccc6)c5c34)n2)cc1 ; c1ccc(-c2nc(-c3ccc4c(ccc5ccccc54)c3)nc(-c3c(-c4ccccc4)ccc4oc5ccccc5c34)n2)cc1 ; c1ccc(-c2nc(-c3ccc4ccc5ccccc5c4c3)nc(-c3cccc4oc5c(-c6ccccc6)cccc5c34)n2)cc1]

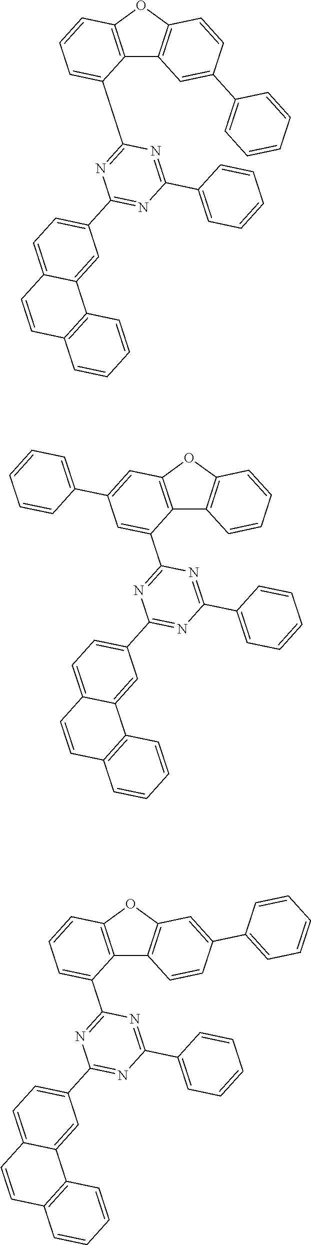 [SMILES: c1ccc(-c2ccc3oc4cccc(-c5nc(-c6ccccc6)nc(-c6ccc7ccc8ccccc8c7c6)n5)c4c3c2)cc1 ; c1ccc(-c2cc(-c3nc(-c4ccccc4)nc(-c4ccc5ccc6ccccc6c5c4)n3)c3c(c2)oc2ccccc23)cc1 ; c1ccc(-c2ccc3c(c2)oc2cccc(-c4nc(-c5ccccc5)nc(-c5ccc6ccc7ccccc7c6c5)n4)c23)cc1]

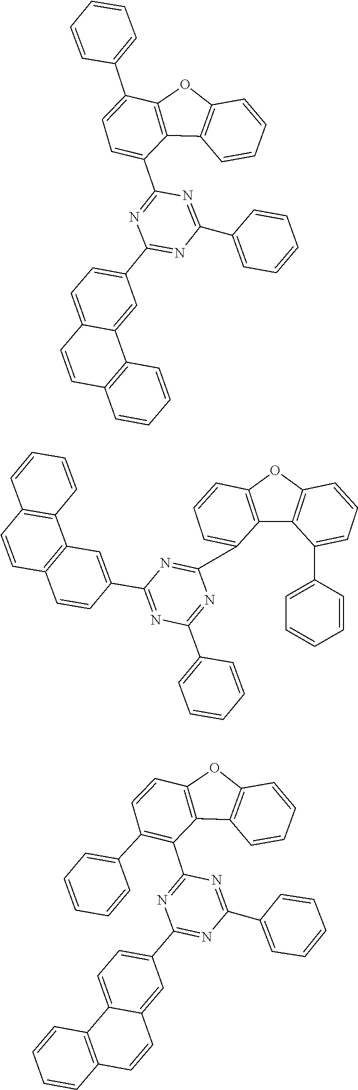 [SMILES: c1ccc(-c2nc(-c3ccc4ccc5ccccc5c4c3)nc(-c3ccc(-c4ccccc4)c4oc5ccccc5c34)n2)cc1 ; c1ccc(-c2nc(-c3ccc4ccc5ccccc5c4c3)nc(-c3cccc4oc5cccc(-c6ccccc6)c5c34)n2)cc1 ; c1ccc(-c2nc(-c3ccc4c(ccc5ccccc54)c3)nc(-c3c(-c4ccccc4)ccc4oc5ccccc5c34)n2)cc1]

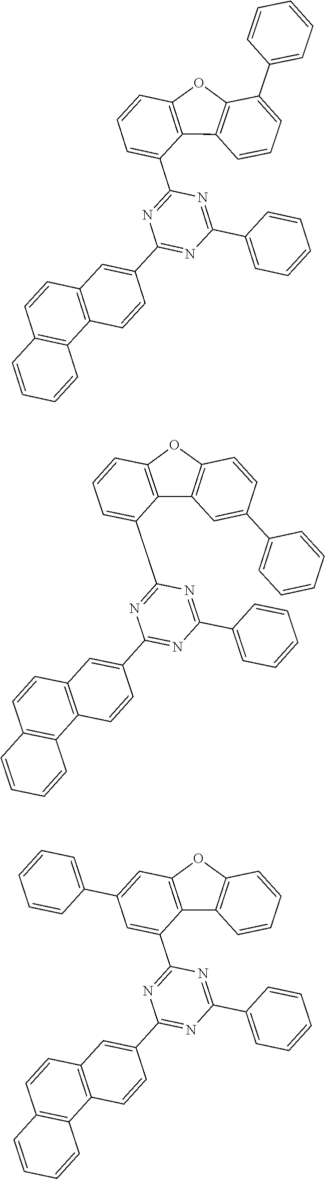 [SMILES: c1ccc(-c2nc(-c3ccc4c(ccc5ccccc54)c3)nc(-c3cccc4oc5c(-c6ccccc6)cccc5c34)n2)cc1 ; c1ccc(-c2ccc3oc4cccc(-c5nc(-c6ccccc6)nc(-c6ccc7c(ccc8ccccc87)c6)n5)c4c3c2)cc1 ; c1ccc(-c2cc(-c3nc(-c4ccccc4)nc(-c4ccc5c(ccc6ccccc65)c4)n3)c3c(c2)oc2ccccc23)cc1]

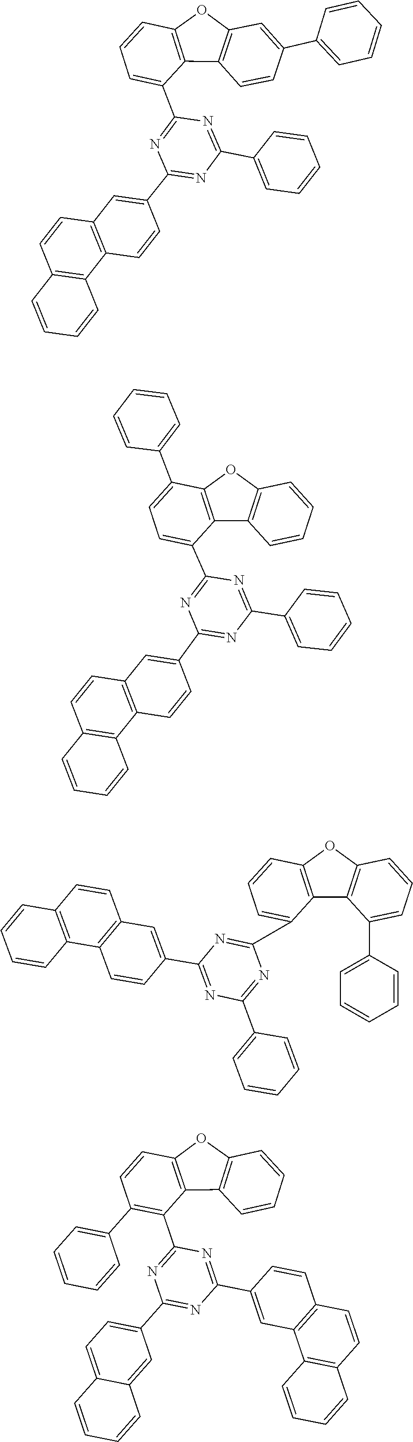 [SMILES: c1ccc(-c2ccc3c(c2)oc2cccc(-c4nc(-c5ccccc5)nc(-c5ccc6c(ccc7ccccc76)c5)n4)c23)cc1 ; c1ccc(-c2nc(-c3ccc4c(ccc5ccccc54)c3)nc(-c3ccc(-c4ccccc4)c4oc5ccccc5c34)n2)cc1 ; c1ccc(-c2nc(-c3ccc4c(ccc5ccccc54)c3)nc(-c3cccc4oc5cccc(-c6ccccc6)c5c34)n2)cc1 ; c1ccc(-c2ccc3oc4ccccc4c3c2-c2nc(-c3ccc4ccccc4c3)nc(-c3ccc4ccc5ccccc5c4c3)n2)cc1]

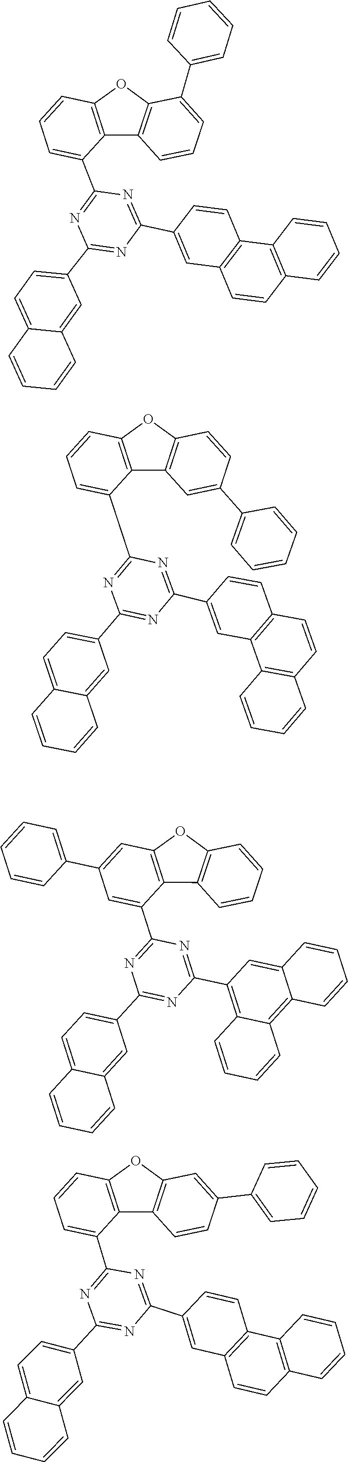

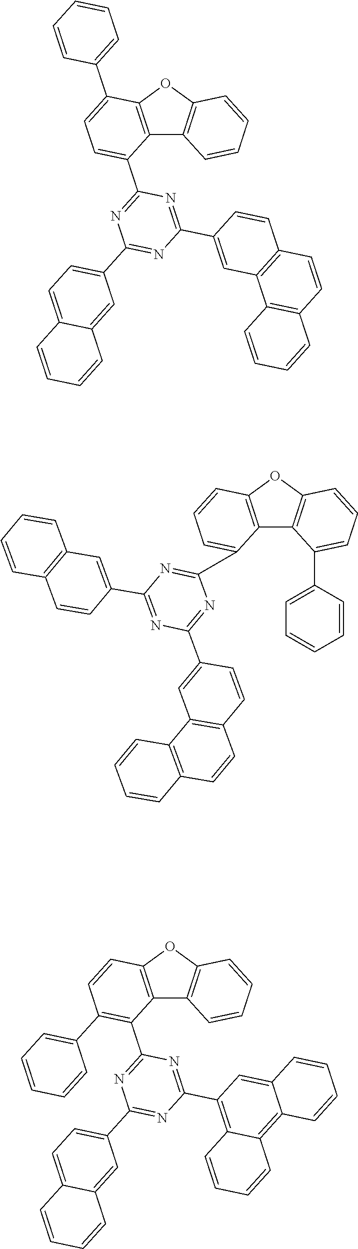 [SMILES: c1ccc(-c2ccc(-c3nc(-c4ccc5ccccc5c4)nc(-c4ccc5ccc6ccccc6c5c4)n3)c3c2oc2ccccc23)cc1 ; c1ccc(-c2cccc3oc4cccc(-c5nc(-c6ccc7ccccc7c6)nc(-c6ccc7ccc8ccccc8c7c6)n5)c4c23)cc1 ; c1ccc(-c2ccc3oc4ccccc4c3c2-c2nc(-c3ccc4ccccc4c3)nc(-c3cc4ccccc4c4ccccc34)n2)cc1]

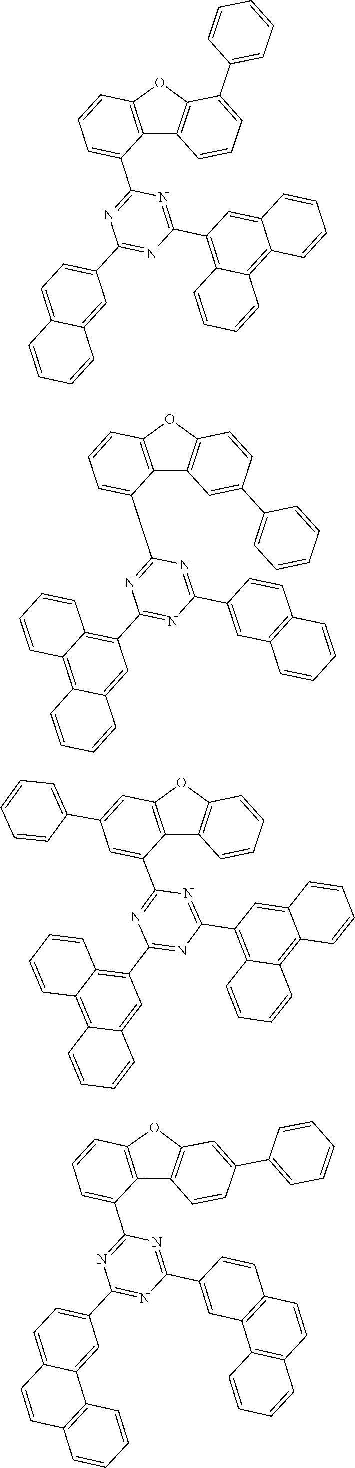

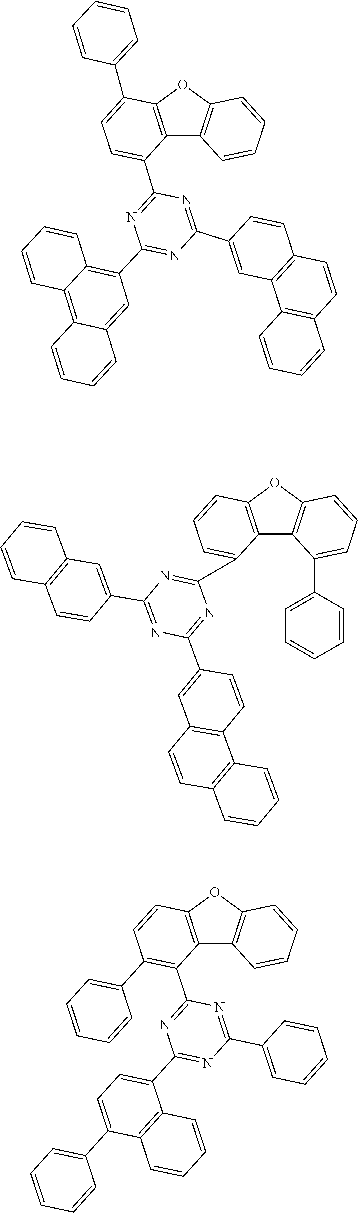 [SMILES: c1ccc(-c2ccc(-c3nc(-c4ccc5ccc6ccccc6c5c4)nc(-c4cc5ccccc5c5ccccc45)n3)c3c2oc2ccccc23)cc1 ; c1ccc(-c2cccc3oc4cccc(-c5nc(-c6ccc7ccccc7c6)nc(-c6ccc7c(ccc8ccccc87)c6)n5)c4c23)cc1 ; c1ccc(-c2nc(-c3ccc(-c4ccccc4)c4ccccc34)nc(-c3c(-c4ccccc4)ccc4oc5ccccc5c34)n2)cc1]

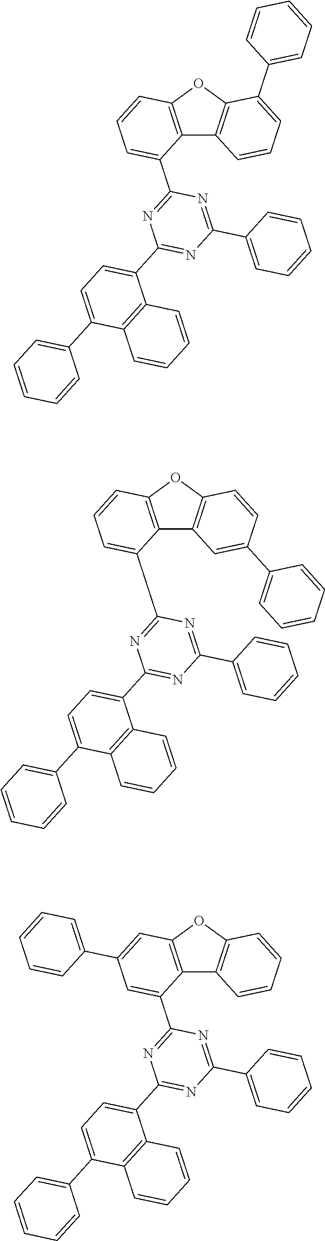 [SMILES: c1ccc(-c2nc(-c3ccc(-c4ccccc4)c4ccccc34)nc(-c3cccc4oc5c(-c6ccccc6)cccc5c34)n2)cc1 ; c1ccc(-c2ccc3oc4cccc(-c5nc(-c6ccccc6)nc(-c6ccc(-c7ccccc7)c7ccccc67)n5)c4c3c2)cc1 ; c1ccc(-c2cc(-c3nc(-c4ccccc4)nc(-c4ccc(-c5ccccc5)c5ccccc45)n3)c3c(c2)oc2ccccc23)cc1]

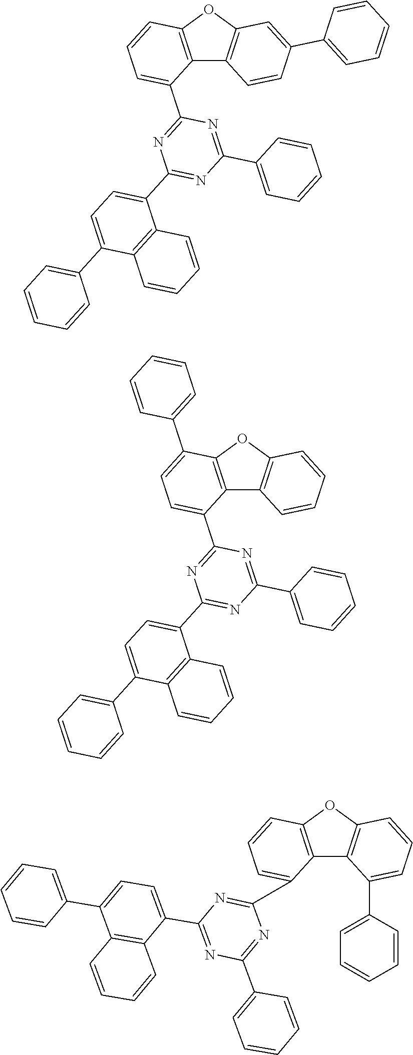 [SMILES: c1ccc(-c2ccc3c(c2)oc2cccc(-c4nc(-c5ccccc5)nc(-c5ccc(-c6ccccc6)c6ccccc56)n4)c23)cc1 ; c1ccc(-c2nc(-c3ccc(-c4ccccc4)c4ccccc34)nc(-c3ccc(-c4ccccc4)c4oc5ccccc5c34)n2)cc1 ; c1ccc(-c2nc(-c3ccc(-c4ccccc4)c4ccccc34)nc(-c3cccc4oc5cccc(-c6ccccc6)c5c34)n2)cc1]

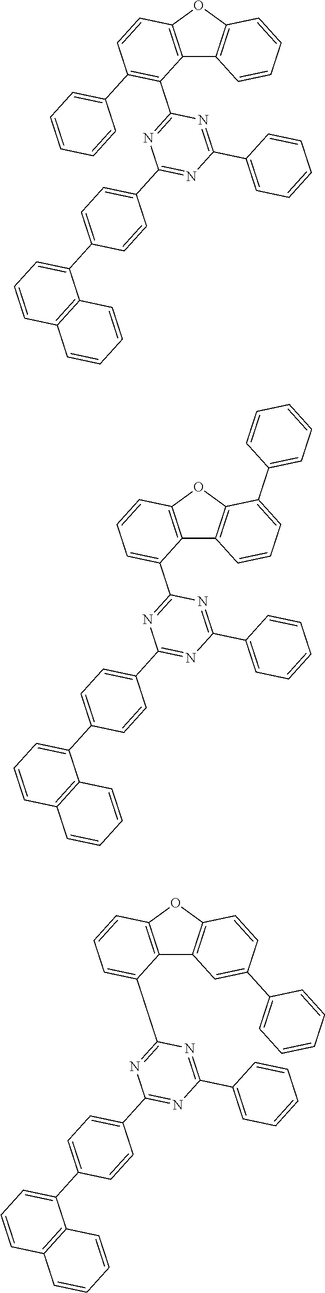 [SMILES: c1ccc(-c2nc(-c3ccc(-c4cccc5ccccc45)cc3)nc(-c3c(-c4ccccc4)ccc4oc5ccccc5c34)n2)cc1 ; c1ccc(-c2nc(-c3ccc(-c4cccc5ccccc45)cc3)nc(-c3cccc4oc5c(-c6ccccc6)cccc5c34)n2)cc1 ; c1ccc(-c2ccc3oc4cccc(-c5nc(-c6ccccc6)nc(-c6ccc(-c7cccc8ccccc78)cc6)n5)c4c3c2)cc1]

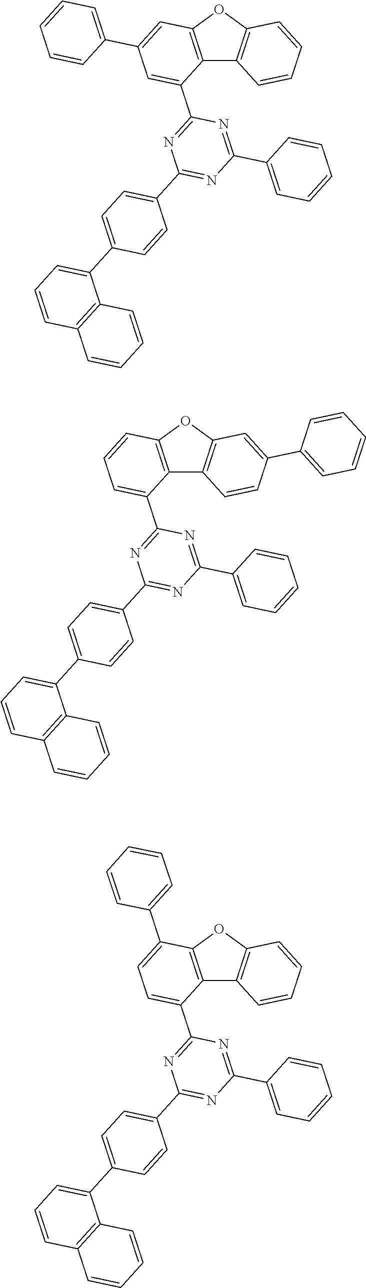 [SMILES: c1ccc(-c2cc(-c3nc(-c4ccccc4)nc(-c4ccc(-c5cccc6ccccc56)cc4)n3)c3c(c2)oc2ccccc23)cc1 ; c1ccc(-c2ccc3c(c2)oc2cccc(-c4nc(-c5ccccc5)nc(-c5ccc(-c6cccc7ccccc67)cc5)n4)c23)cc1 ; c1ccc(-c2nc(-c3ccc(-c4cccc5ccccc45)cc3)nc(-c3ccc(-c4ccccc4)c4oc5ccccc5c34)n2)cc1]

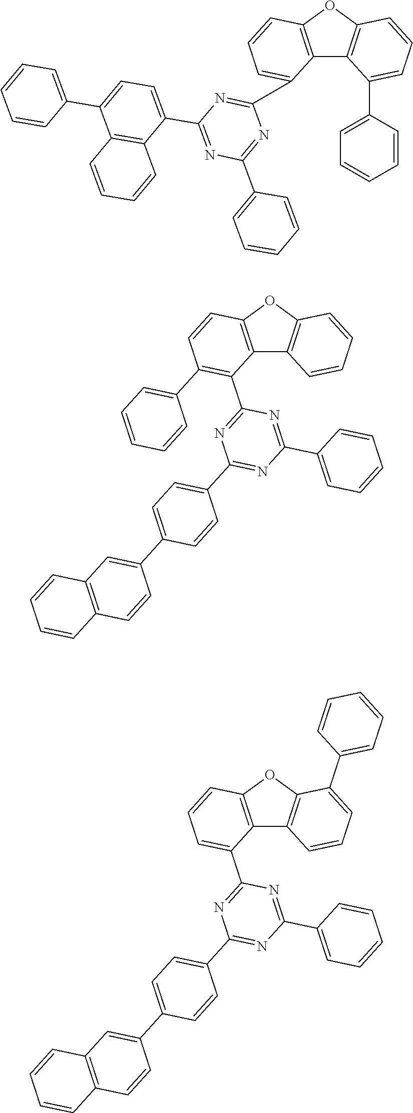

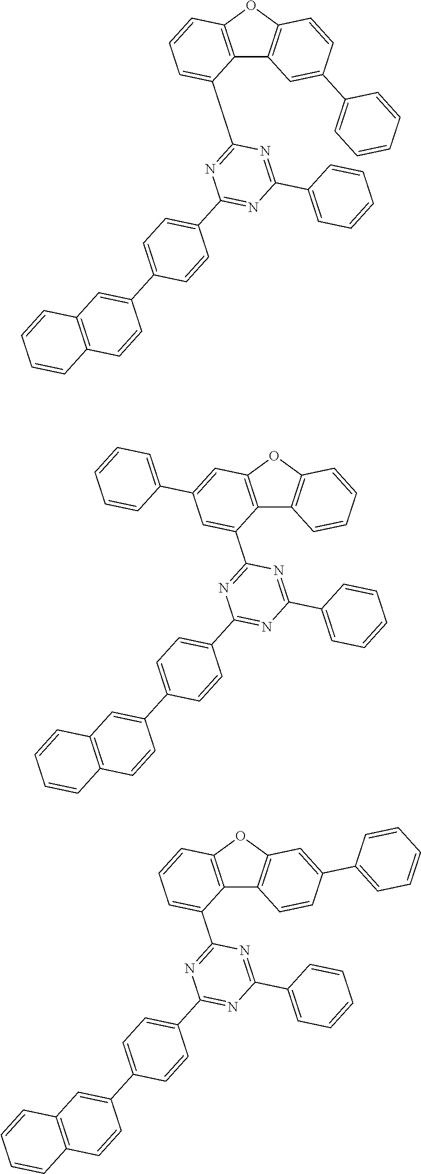 [SMILES: c1ccc(-c2ccc3oc4cccc(-c5nc(-c6ccccc6)nc(-c6ccc(-c7ccc8ccccc8c7)cc6)n5)c4c3c2)cc1 ; c1ccc(-c2cc(-c3nc(-c4ccccc4)nc(-c4ccc(-c5ccc6ccccc6c5)cc4)n3)c3c(c2)oc2ccccc23)cc1 ; c1ccc(-c2ccc3c(c2)oc2cccc(-c4nc(-c5ccccc5)nc(-c5ccc(-c6ccc7ccccc7c6)cc5)n4)c23)cc1]

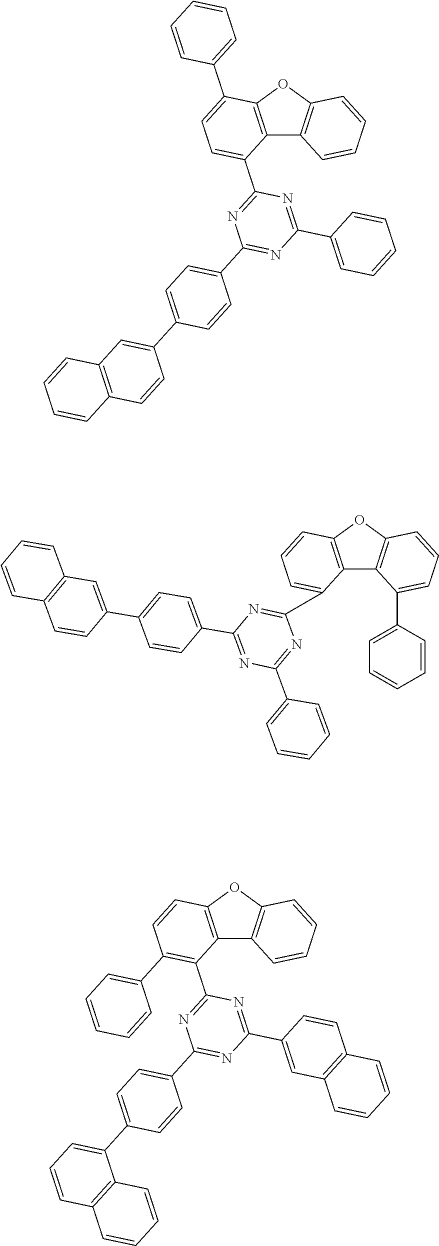 [SMILES: c1ccc(-c2nc(-c3ccc(-c4ccc5ccccc5c4)cc3)nc(-c3ccc(-c4ccccc4)c4oc5ccccc5c34)n2)cc1 ; c1ccc(-c2nc(-c3ccc(-c4ccc5ccccc5c4)cc3)nc(-c3cccc4oc5cccc(-c6ccccc6)c5c34)n2)cc1 ; c1ccc(-c2ccc3oc4ccccc4c3c2-c2nc(-c3ccc(-c4cccc5ccccc45)cc3)nc(-c3ccc4ccccc4c3)n2)cc1]

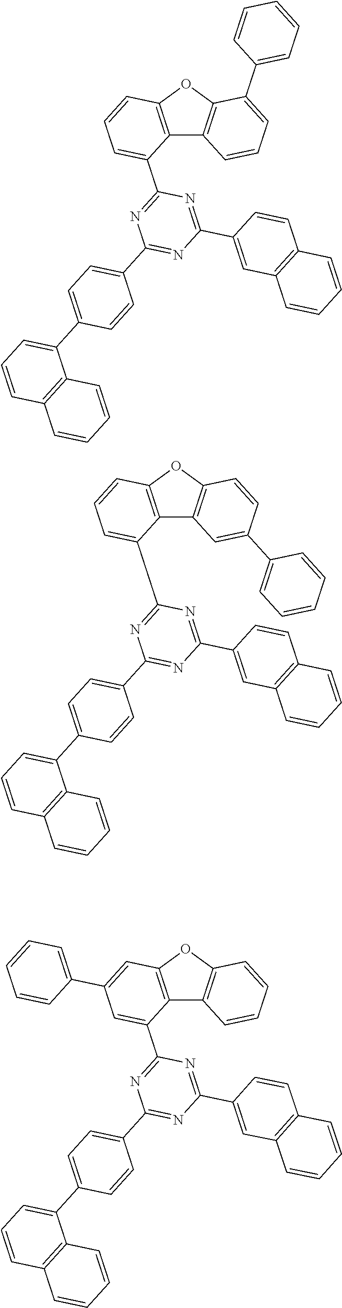 [SMILES: c1ccc(-c2cccc3c2oc2cccc(-c4nc(-c5ccc(-c6cccc7ccccc67)cc5)nc(-c5ccc6ccccc6c5)n4)c23)cc1 ; c1ccc(-c2ccc3oc4cccc(-c5nc(-c6ccc(-c7cccc8ccccc78)cc6)nc(-c6ccc7ccccc7c6)n5)c4c3c2)cc1 ; c1ccc(-c2cc(-c3nc(-c4ccc(-c5cccc6ccccc56)cc4)nc(-c4ccc5ccccc5c4)n3)c3c(c2)oc2ccccc23)cc1]

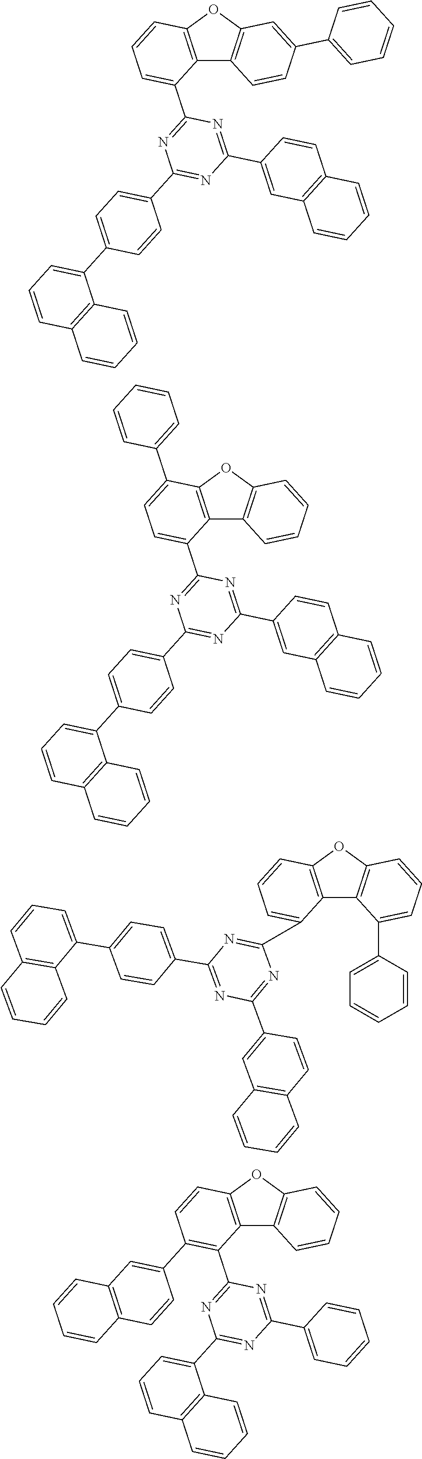

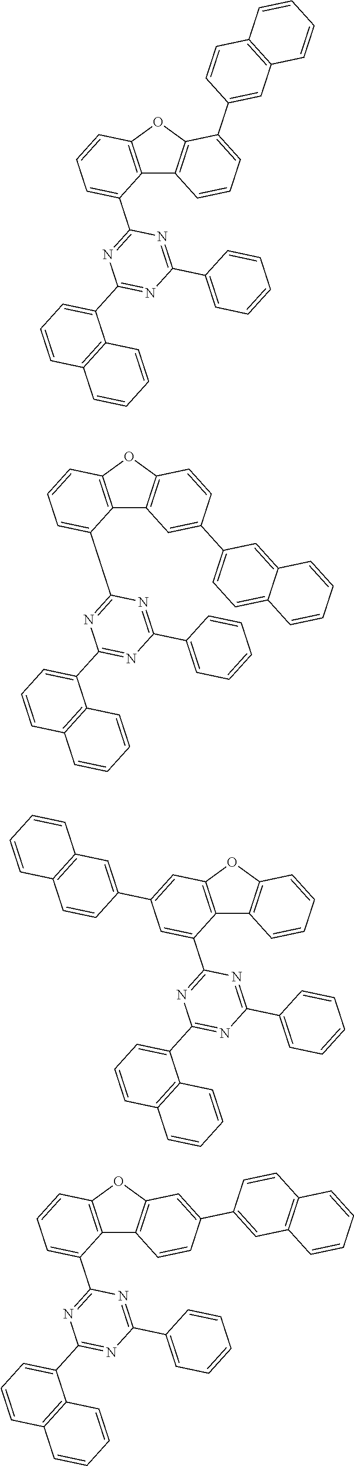 [SMILES: c1ccc(-c2nc(-c3cccc4ccccc34)nc(-c3cccc4oc5c(-c6ccc7ccccc7c6)cccc5c34)n2)cc1 ; c1ccc(-c2nc(-c3cccc4ccccc34)nc(-c3cccc4oc5ccc(-c6ccc7ccccc7c6)cc5c34)n2)cc1 ; c1ccc(-c2nc(-c3cccc4ccccc34)nc(-c3cc(-c4ccc5ccccc5c4)cc4oc5ccccc5c34)n2)cc1 ; c1ccc(-c2nc(-c3cccc4ccccc34)nc(-c3cccc4oc5cc(-c6ccc7ccccc7c6)ccc5c34)n2)cc1]

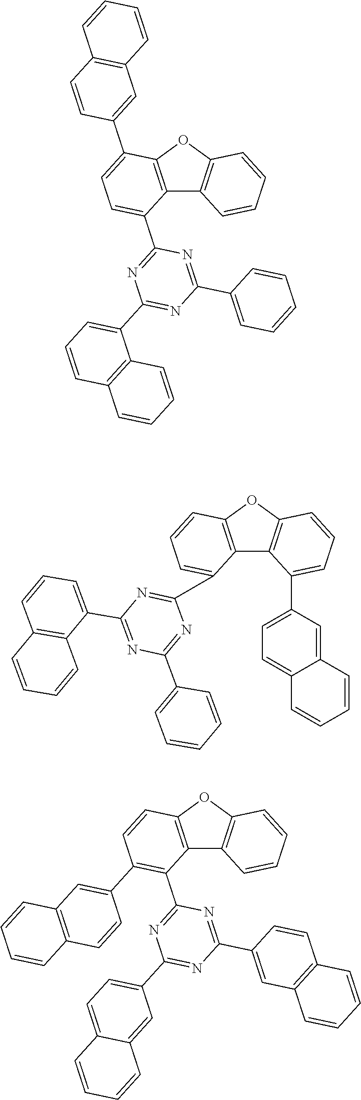 [SMILES: c1ccc(-c2nc(-c3cccc4ccccc34)nc(-c3ccc(-c4ccc5ccccc5c4)c4oc5ccccc5c34)n2)cc1 ; c1ccc(-c2nc(-c3cccc4ccccc34)nc(-c3cccc4oc5cccc(-c6ccc7ccccc7c6)c5c34)n2)cc1 ; c1ccc2cc(-c3nc(-c4ccc5ccccc5c4)nc(-c4c(-c5ccc6ccccc6c5)ccc5oc6ccccc6c45)n3)ccc2c1]

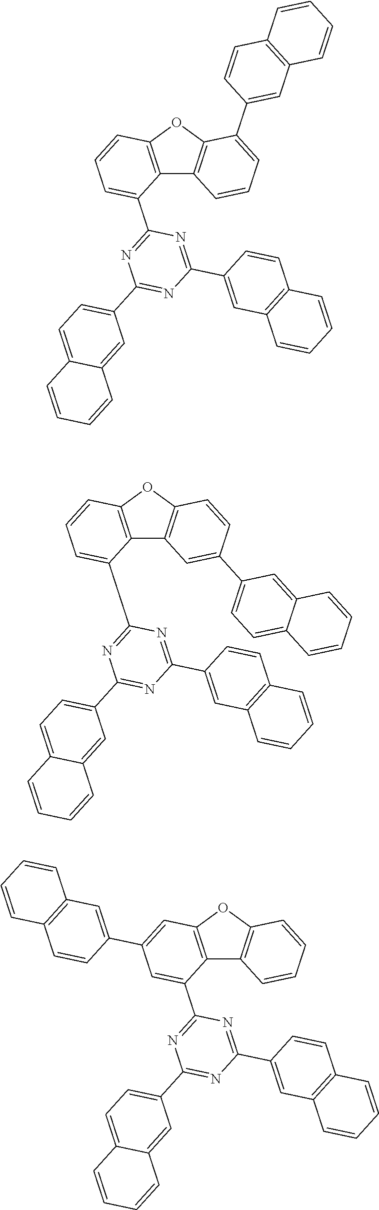 [SMILES: c1ccc2cc(-c3nc(-c4ccc5ccccc5c4)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7ccc8ccccc8c7)n6)c5c4c3)ccc2c1 ; c1ccc2cc(-c3cc(-c4nc(-c5ccc6ccccc6c5)nc(-c5ccc6ccccc6c5)n4)c4c(c3)oc3ccccc34)ccc2c1]

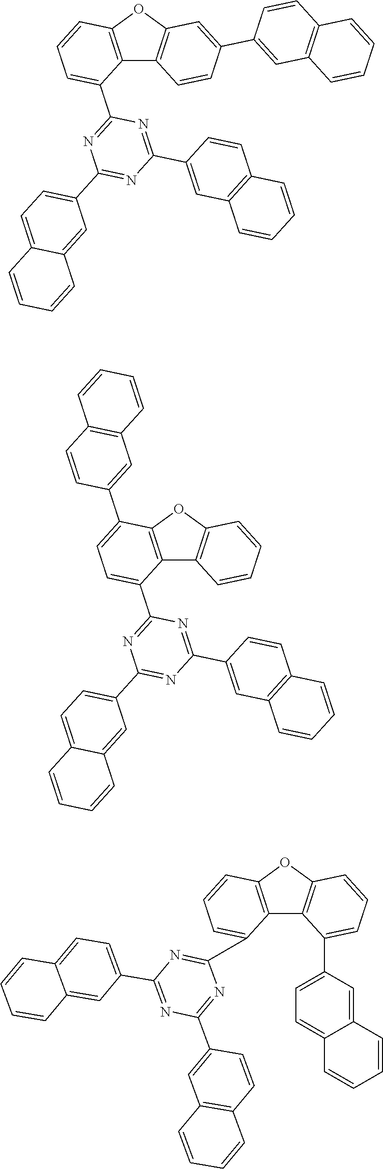 [SMILES: c1ccc2cc(-c3ccc4c(c3)oc3cccc(-c5nc(-c6ccc7ccccc7c6)nc(-c6ccc7ccccc7c6)n5)c34)ccc2c1 ; c1ccc2cc(-c3nc(-c4ccc5ccccc5c4)nc(-c4ccc(-c5ccc6ccccc6c5)c5oc6ccccc6c45)n3)ccc2c1 ; c1ccc2cc(-c3nc(-c4ccc5ccccc5c4)nc(-c4cccc5oc6cccc(-c7ccc8ccccc8c7)c6c45)n3)ccc2c1]

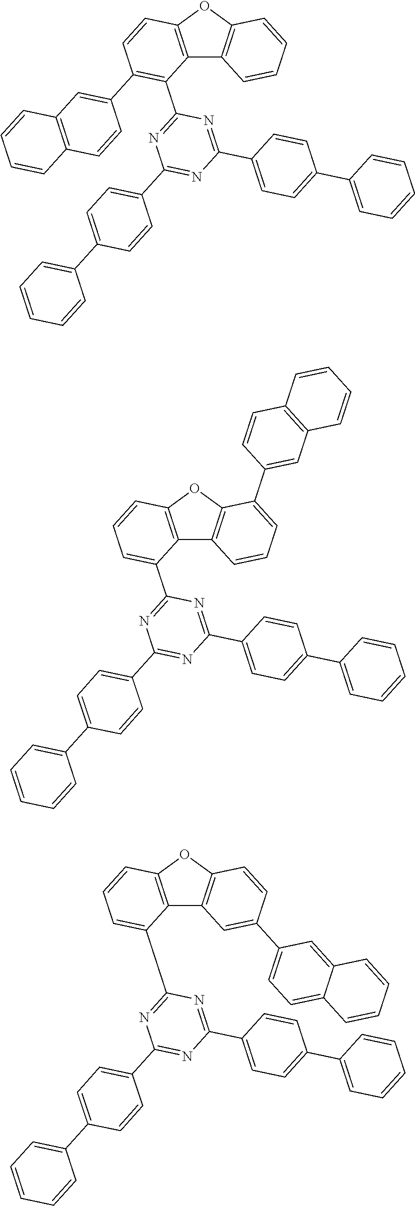 [SMILES: c1ccc(-c2ccc(-c3nc(-c4ccc(-c5ccccc5)cc4)nc(-c4c(-c5ccc6ccccc6c5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc8ccccc8c7)cccc6c45)n3)cc2)cc1 ; c1ccc(-c2ccc(-c3nc(-c4ccc(-c5ccccc5)cc4)nc(-c4cccc5oc6ccc(-c7ccc8ccccc8c7)cc6c45)n3)cc2)cc1]

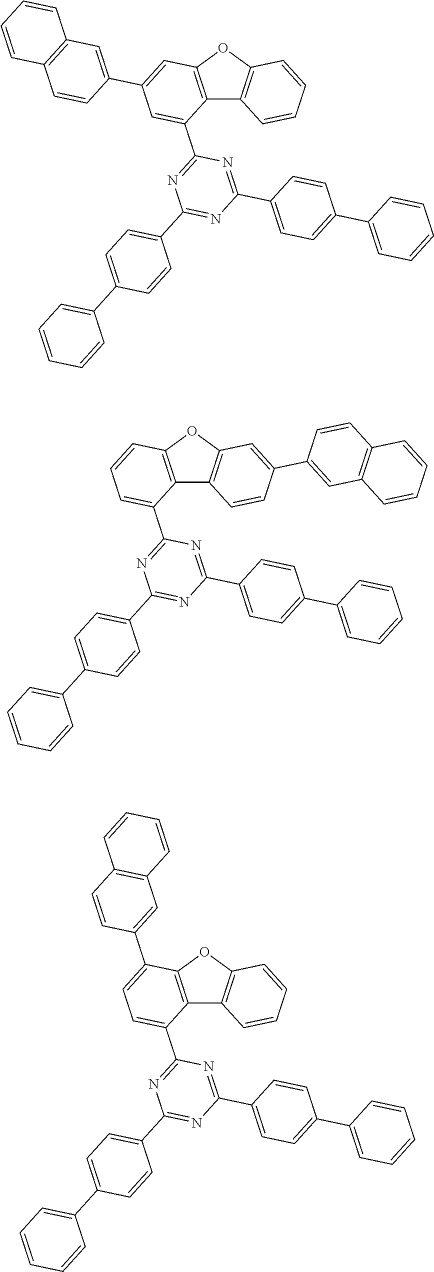 [SMILES: c1ccc(-c2ccc(-c3nc(-c4ccc(-c5ccccc5)cc4)nc(-c4cc(-c5ccc6ccccc6c5)cc5oc6ccccc6c45)n3)cc2)cc1 ; c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1]

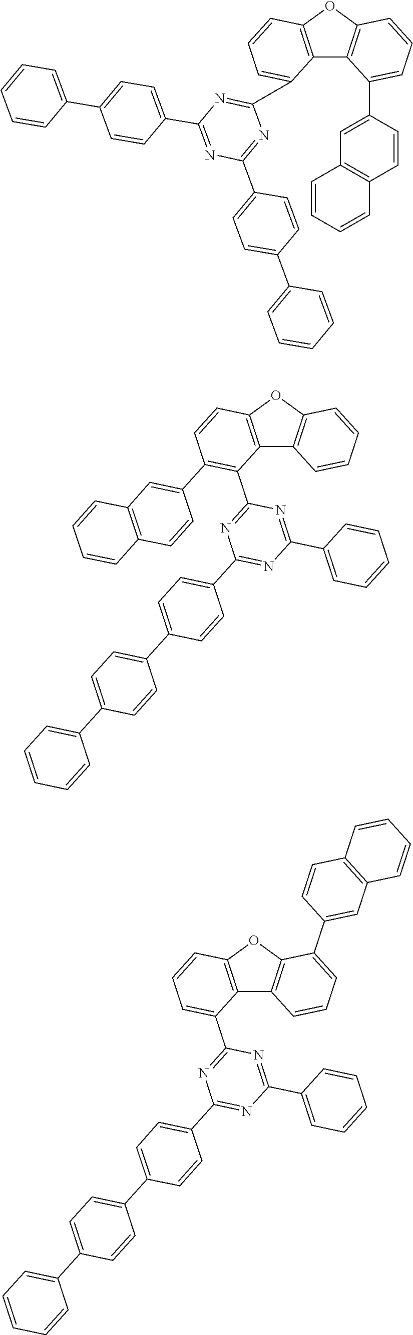 [SMILES: c1ccc(-c2ccc(-c3nc(-c4ccc(-c5ccccc5)cc4)nc(-c4cccc5oc6cccc(-c7ccc8ccccc8c7)c6c45)n3)cc2)cc1 ; c1ccc(-c2ccc(-c3ccc(-c4nc(-c5ccccc5)nc(-c5c(-c6ccc7ccccc7c6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1]

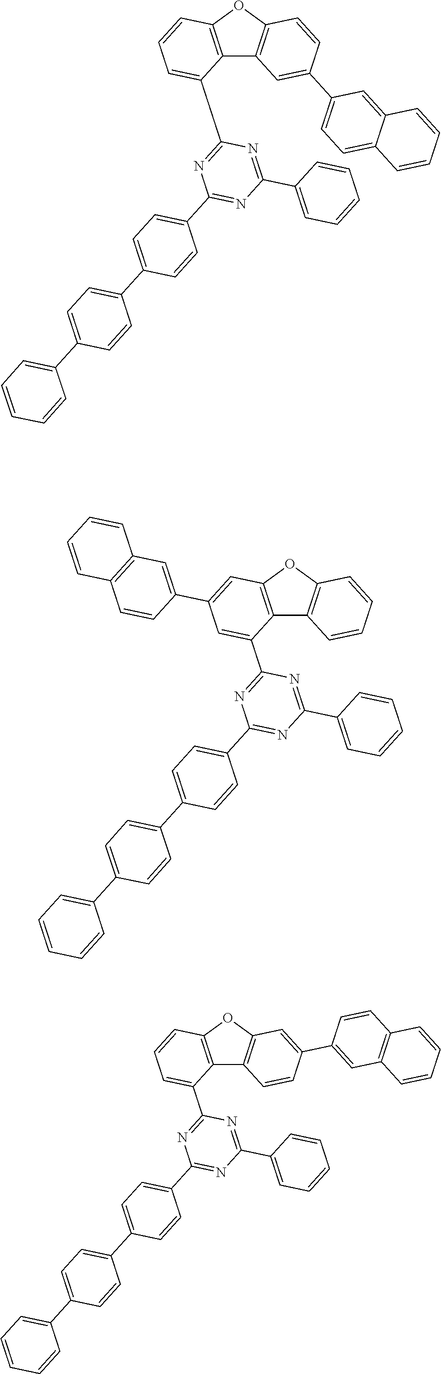 [SMILES: c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1]

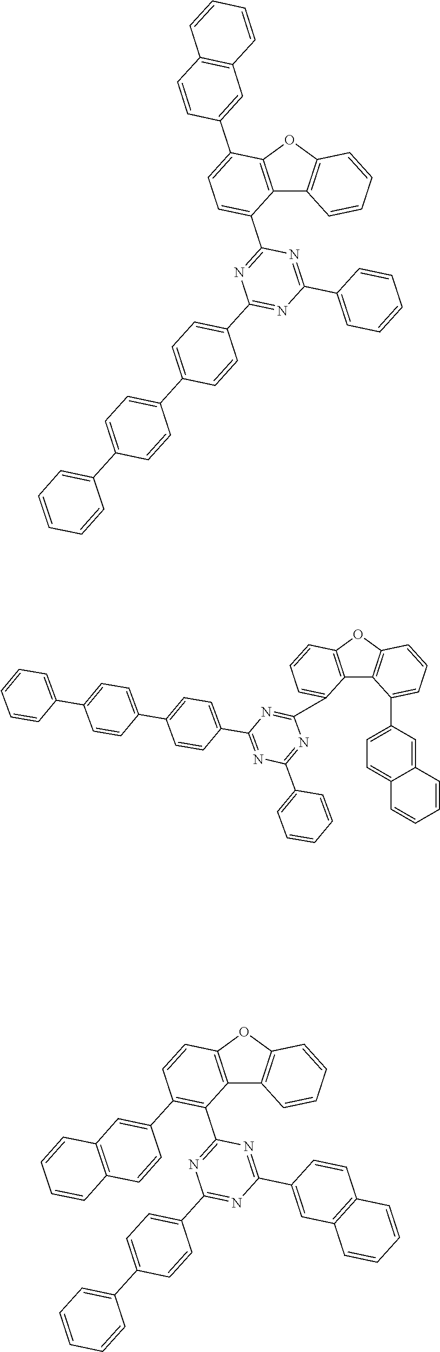 [SMILES: c1ccc(-c2ccc(-c3ccc(-c4nc(-c5ccccc5)nc(-c5ccc(-c6ccc7ccccc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3nc(-c4ccc5ccccc5c4)nc(-c4c(-c5ccc6ccccc6c5)ccc5oc6ccccc6c45)n3)cc2)cc1]

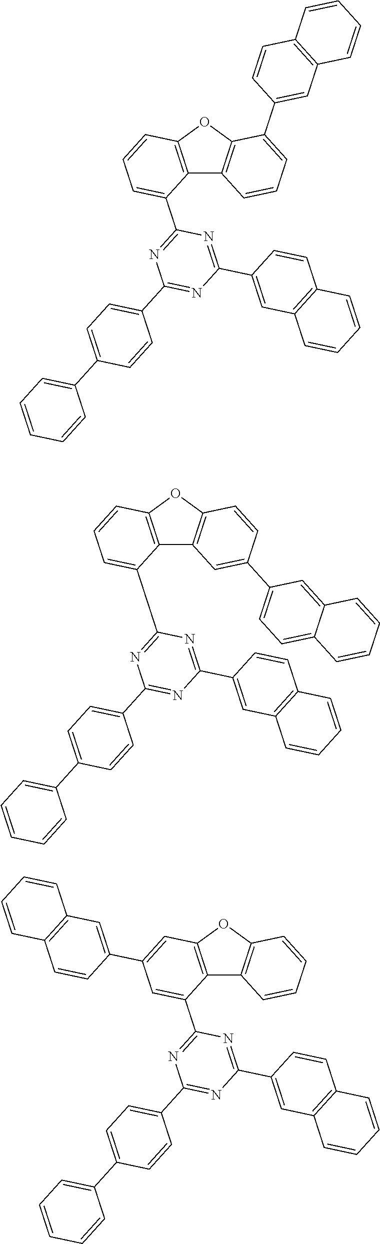

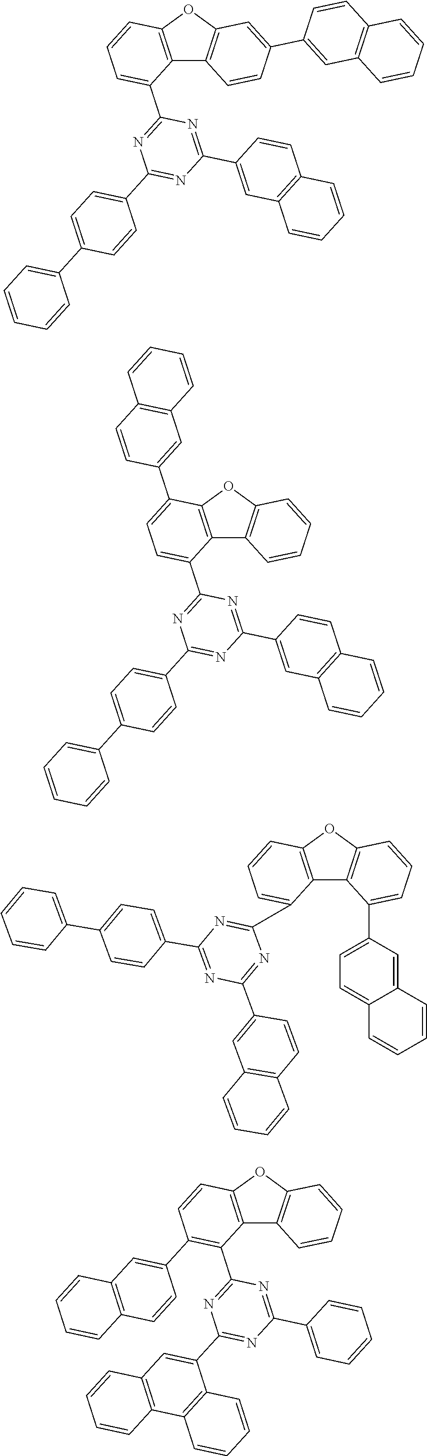

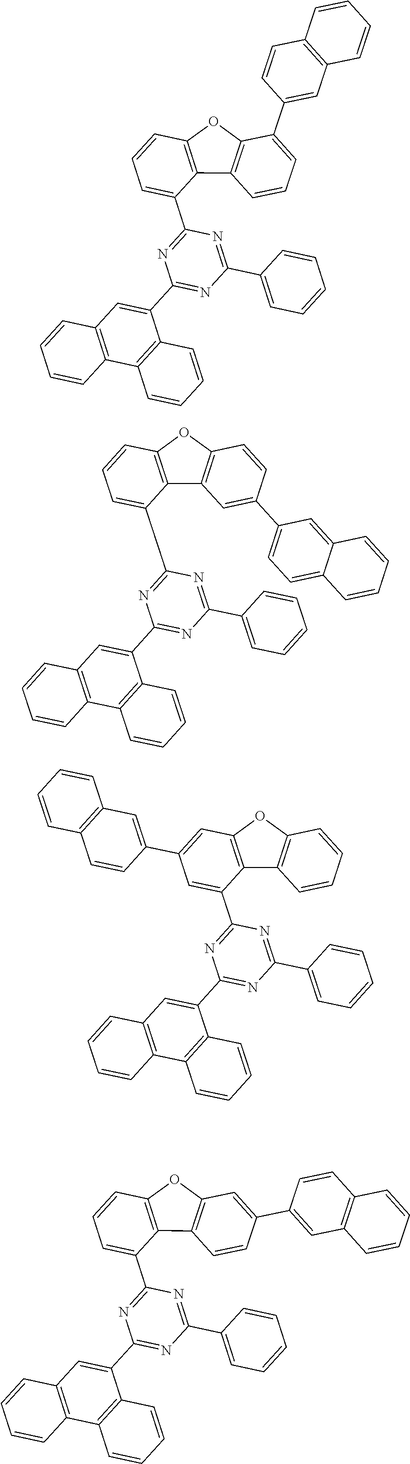

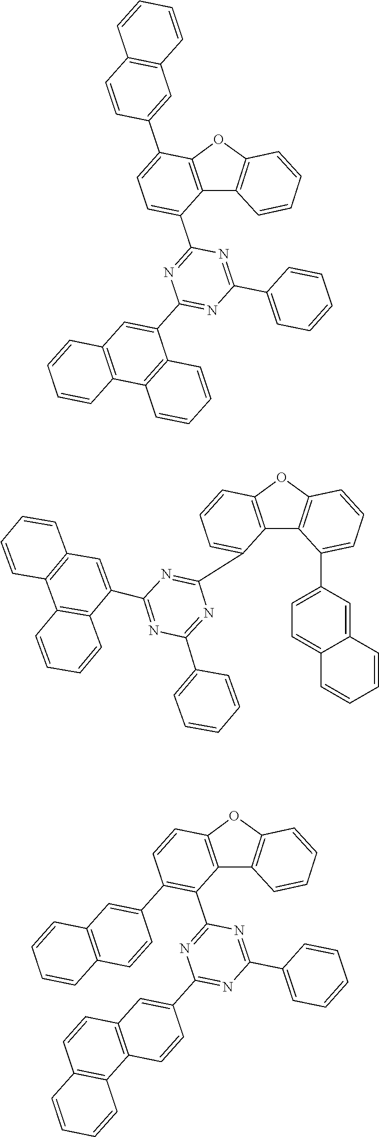 [SMILES: c1ccc(-c2nc(-c3cc4ccccc4c4ccccc34)nc(-c3ccc(-c4ccc5ccccc5c4)c4oc5ccccc5c34)n2)cc1 ; c1ccc(-c2nc(-c3cc4ccccc4c4ccccc34)nc(-c3cccc4oc5cccc(-c6ccc7ccccc7c6)c5c34)n2)cc1 ; c1ccc(-c2nc(-c3ccc4c(ccc5ccccc54)c3)nc(-c3c(-c4ccc5ccccc5c4)ccc4oc5ccccc5c34)n2)cc1]

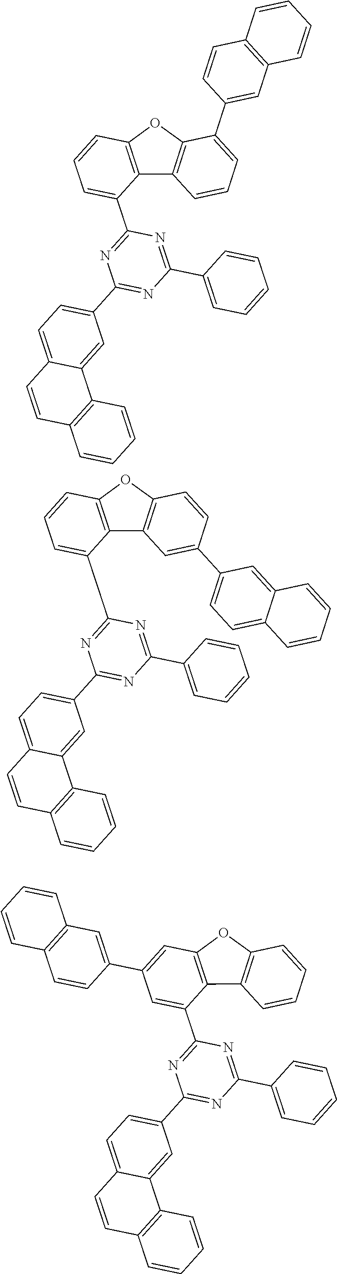

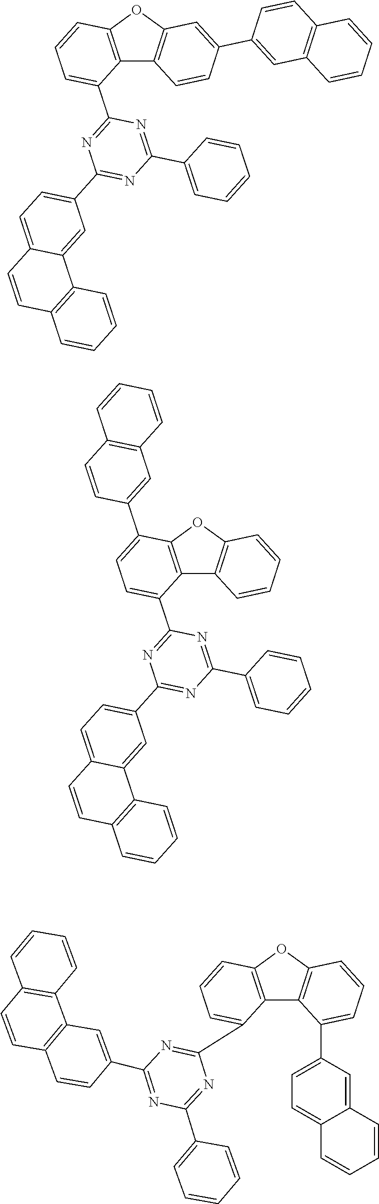

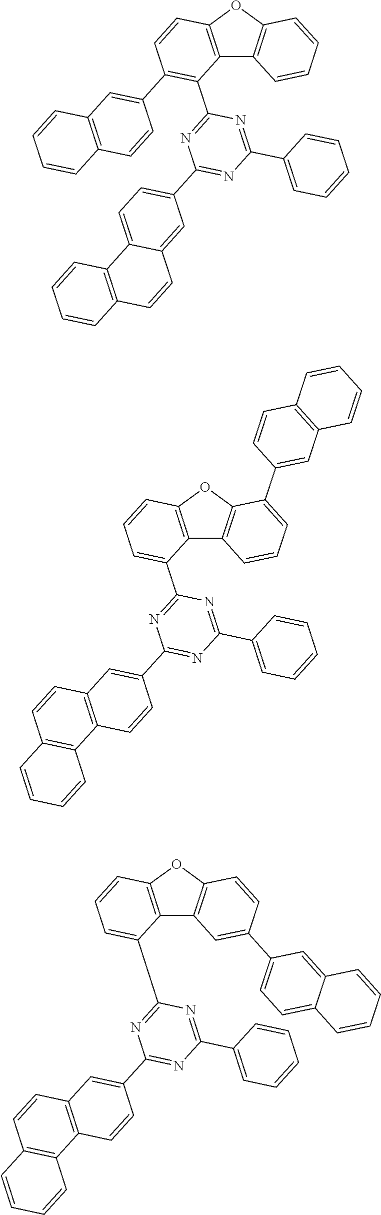 [SMILES: c1ccc(-c2nc(-c3ccc4c(ccc5ccccc54)c3)nc(-c3c(-c4ccc5ccccc5c4)ccc4oc5ccccc5c34)n2)cc1 ; c1ccc(-c2nc(-c3ccc4c(ccc5ccccc54)c3)nc(-c3cccc4oc5c(-c6ccc7ccccc7c6)cccc5c34)n2)cc1 ; c1ccc(-c2nc(-c3ccc4c(ccc5ccccc54)c3)nc(-c3cccc4oc5ccc(-c6ccc7ccccc7c6)cc5c34)n2)cc1]

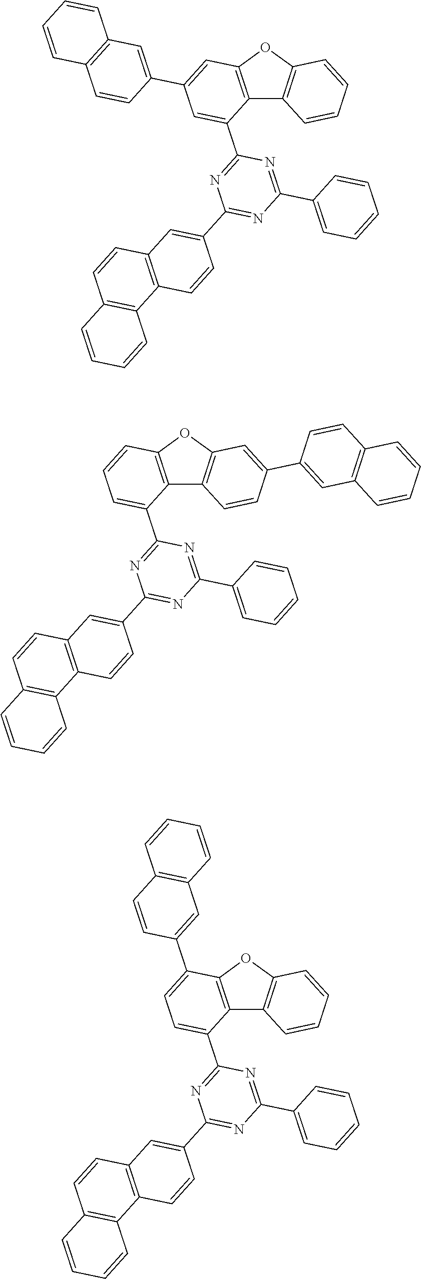 [SMILES: c1ccc(-c2nc(-c3ccc4c(ccc5ccccc54)c3)nc(-c3cc(-c4ccc5ccccc5c4)cc4oc5ccccc5c34)n2)cc1 ; c1ccc(-c2nc(-c3ccc4c(ccc5ccccc54)c3)nc(-c3cccc4oc5cc(-c6ccc7ccccc7c6)ccc5c34)n2)cc1 ; c1ccc(-c2nc(-c3ccc4c(ccc5ccccc54)c3)nc(-c3ccc(-c4ccc5ccccc5c4)c4oc5ccccc5c34)n2)cc1]

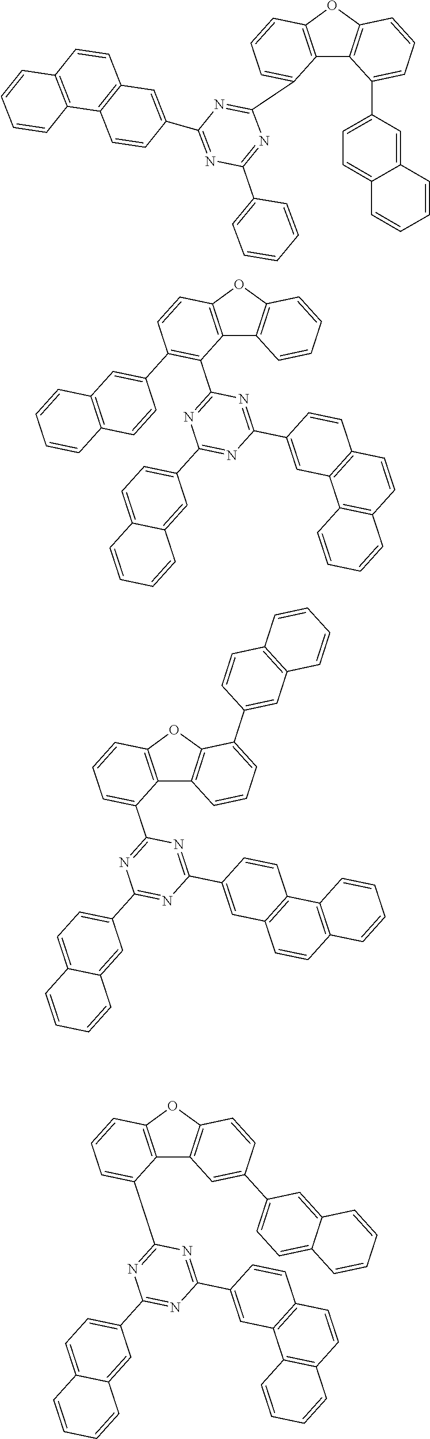

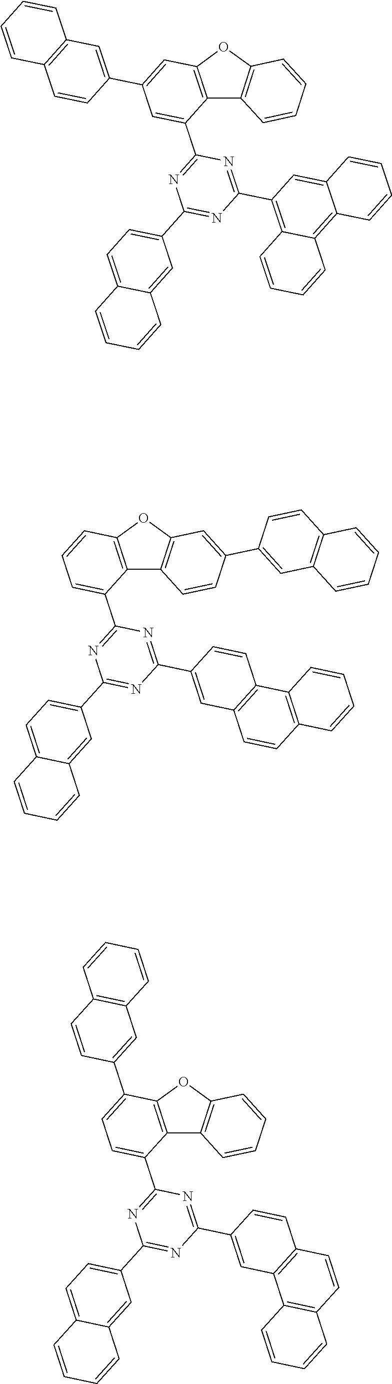 [SMILES: c1ccc2cc(-c3cc(-c4nc(-c5ccc6ccccc6c5)nc(-c5cc6ccccc6c6ccccc56)n4)c4c(c3)oc3ccccc34)ccc2c1 ; c1ccc2cc(-c3ccc4c(c3)oc3cccc(-c5nc(-c6ccc7ccccc7c6)nc(-c6ccc7c(ccc8ccccc87)c6)n5)c34)ccc2c1 ; c1ccc2cc(-c3nc(-c4ccc5ccc6ccccc6c5c4)nc(-c4ccc(-c5ccc6ccccc6c5)c5oc6ccccc6c45)n3)ccc2c1]

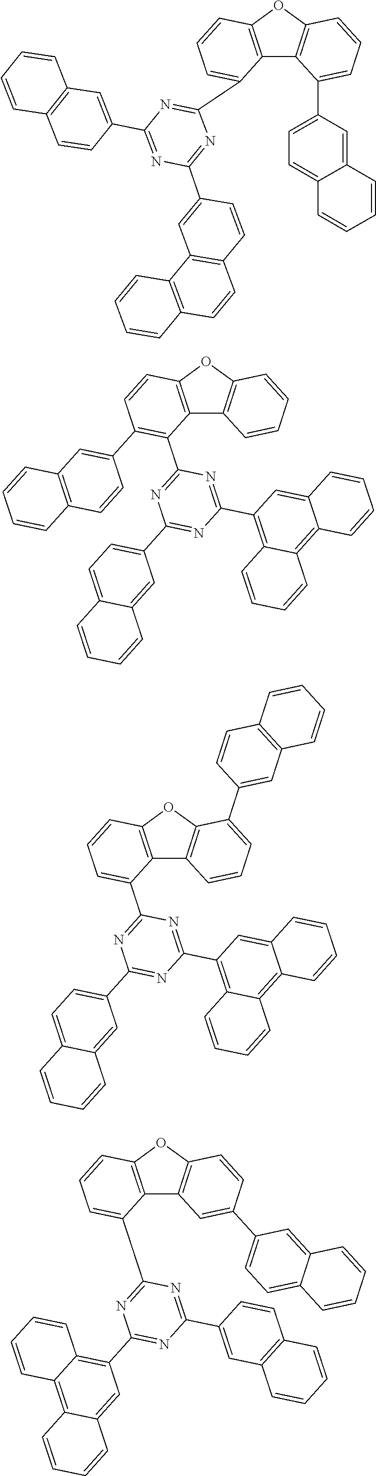

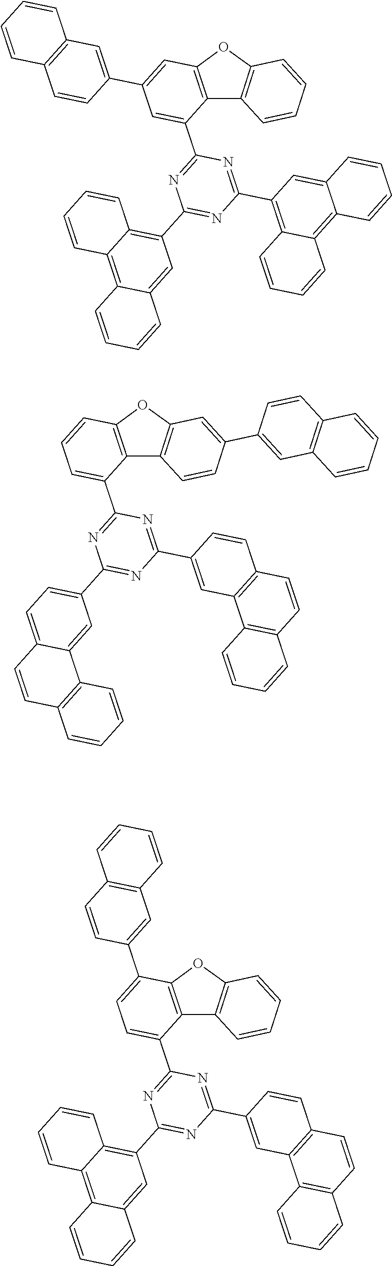 [SMILES: c1ccc2cc(-c3cc(-c4nc(-c5cc6ccccc6c6ccccc56)nc(-c5cc6ccccc6c6ccccc56)n4)c4c(c3)oc3ccccc34)ccc2c1 ; c1ccc2cc(-c3ccc4c(c3)oc3cccc(-c5nc(-c6ccc7ccc8ccccc8c7c6)nc(-c6ccc7ccc8ccccc8c7c6)n5)c34)ccc2c1 ; c1ccc2cc(-c3ccc(-c4nc(-c5ccc6ccc7ccccc7c6c5)nc(-c5cc6ccccc6c6ccccc56)n4)c4c3oc3ccccc34)ccc2c1]

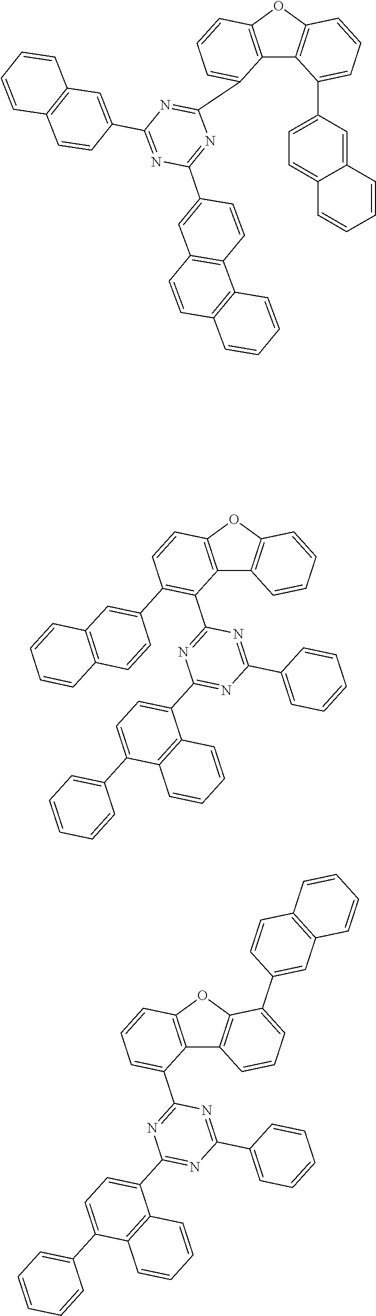 [SMILES: c1ccc2cc(-c3nc(-c4ccc5c(ccc6ccccc65)c4)nc(-c4cccc5oc6cccc(-c7ccc8ccccc8c7)c6c45)n3)ccc2c1 ; c1ccc(-c2nc(-c3ccc(-c4ccccc4)c4ccccc34)nc(-c3c(-c4ccc5ccccc5c4)ccc4oc5ccccc5c34)n2)cc1 ; c1ccc(-c2nc(-c3ccc(-c4ccccc4)c4ccccc34)nc(-c3cccc4oc5c(-c6ccc7ccccc7c6)cccc5c34)n2)cc1]

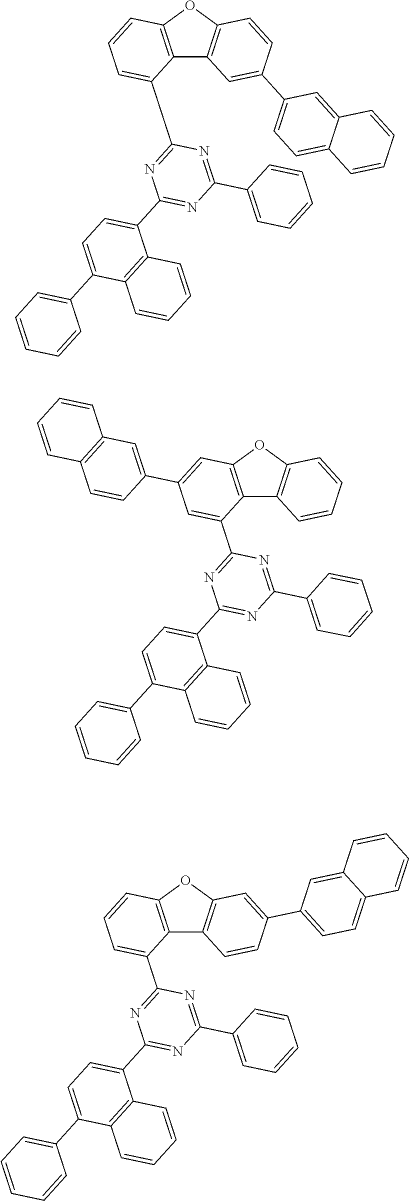

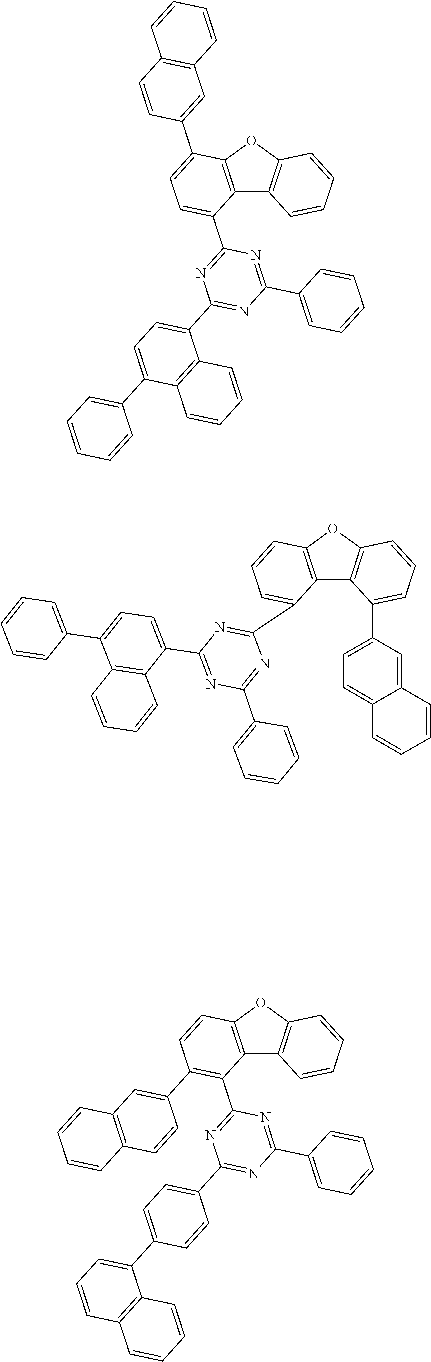 [SMILES: c1ccc(-c2nc(-c3ccc(-c4ccccc4)c4ccccc34)nc(-c3ccc(-c4ccc5ccccc5c4)c4oc5ccccc5c34)n2)cc1 ; c1ccc(-c2nc(-c3ccc(-c4ccccc4)c4ccccc34)nc(-c3cccc4oc5cccc(-c6ccc7ccccc7c6)c5c34)n2)cc1 ; c1ccc(-c2nc(-c3ccc(-c4cccc5ccccc45)cc3)nc(-c3c(-c4ccc5ccccc5c4)ccc4oc5ccccc5c34)n2)cc1]

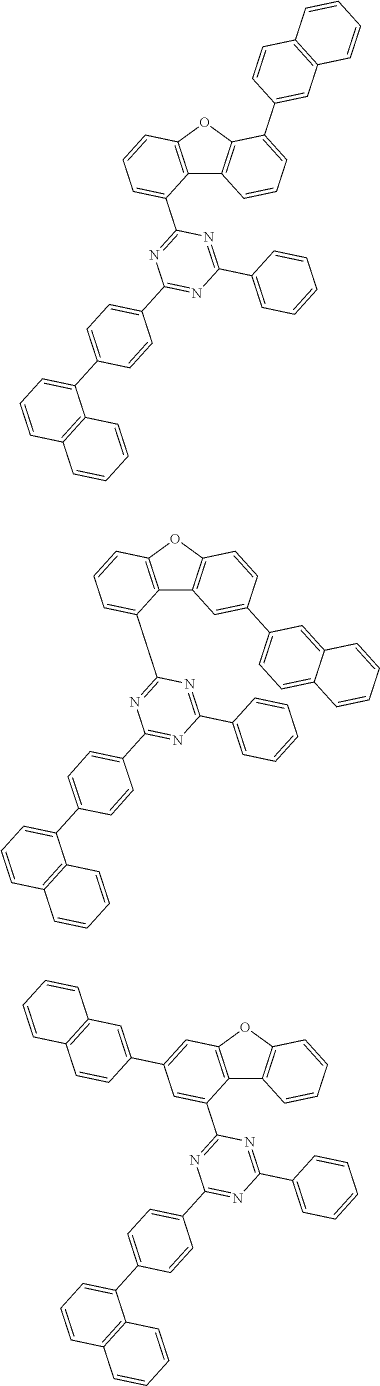 [SMILES: c1ccc(-c2nc(-c3ccc(-c4cccc5ccccc45)cc3)nc(-c3cccc4oc5c(-c6ccc7ccccc7c6)cccc5c34)n2)cc1 ; c1ccc(-c2nc(-c3ccc(-c4cccc5ccccc45)cc3)nc(-c3cccc4oc5ccc(-c6ccc7ccccc7c6)cc5c34)n2)cc1 ; c1ccc(-c2nc(-c3ccc(-c4cccc5ccccc45)cc3)nc(-c3cc(-c4ccc5ccccc5c4)cc4oc5ccccc5c34)n2)cc1]

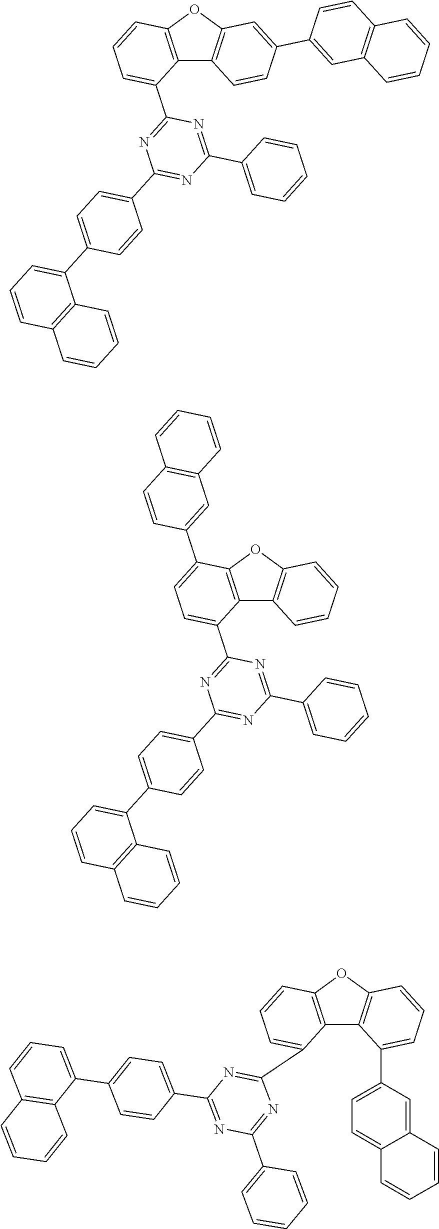 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2nc(-c3ccc(-c4cccc5ccccc45)cc3)nc(-c3ccc(-c4ccc5ccccc5c4)c4oc5ccccc5c34)n2)cc1 ; c1ccc(-c2nc(-c3ccc(-c4cccc5ccccc45)cc3)nc(-c3cccc4oc5cccc(-c6ccc7ccccc7c6)c5c34)n2)cc1]

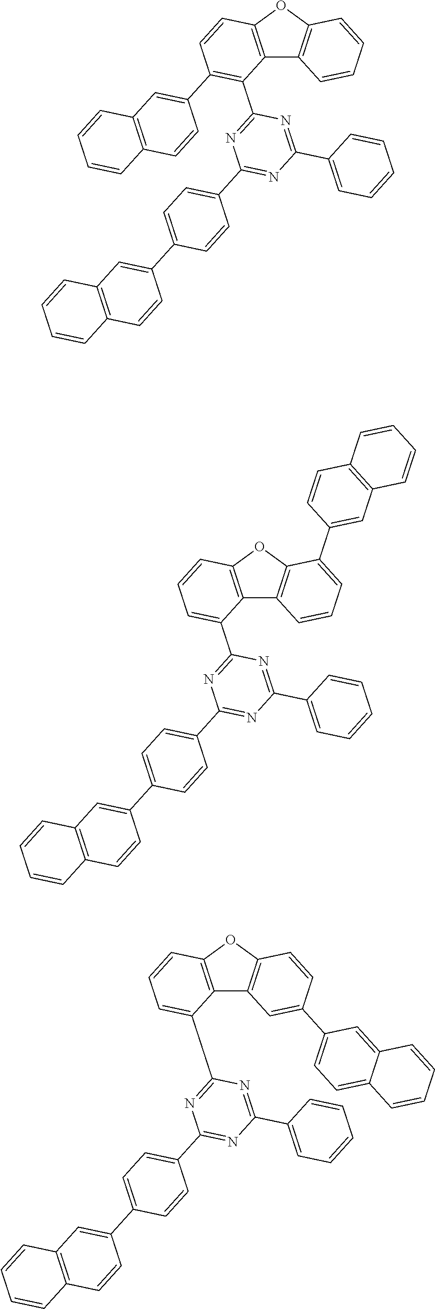 [SMILES: c1ccc(-c2nc(-c3ccc(-c4ccc5ccccc5c4)cc3)nc(-c3c(-c4ccc5ccccc5c4)ccc4oc5ccccc5c34)n2)cc1 ; c1ccc(-c2nc(-c3ccc(-c4ccc5ccccc5c4)cc3)nc(-c3cccc4oc5c(-c6ccc7ccccc7c6)cccc5c34)n2)cc1 ; c1ccc(-c2nc(-c3ccc(-c4ccc5ccccc5c4)cc3)nc(-c3cccc4oc5ccc(-c6ccc7ccccc7c6)cc5c34)n2)cc1]

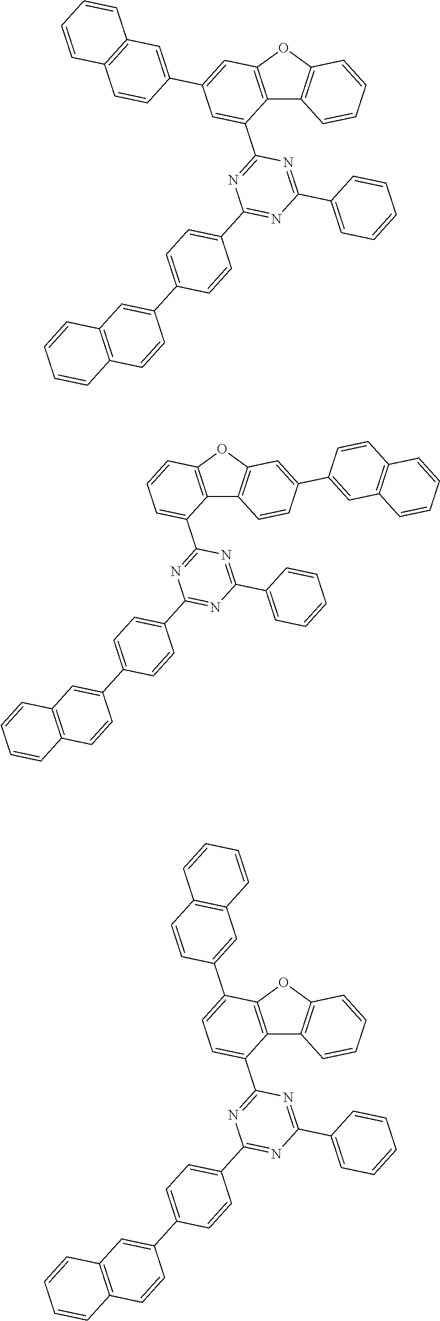 [SMILES: c1ccc(-c2nc(-c3ccc(-c4ccc5ccccc5c4)cc3)nc(-c3cc(-c4ccc5ccccc5c4)cc4oc5ccccc5c34)n2)cc1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1]

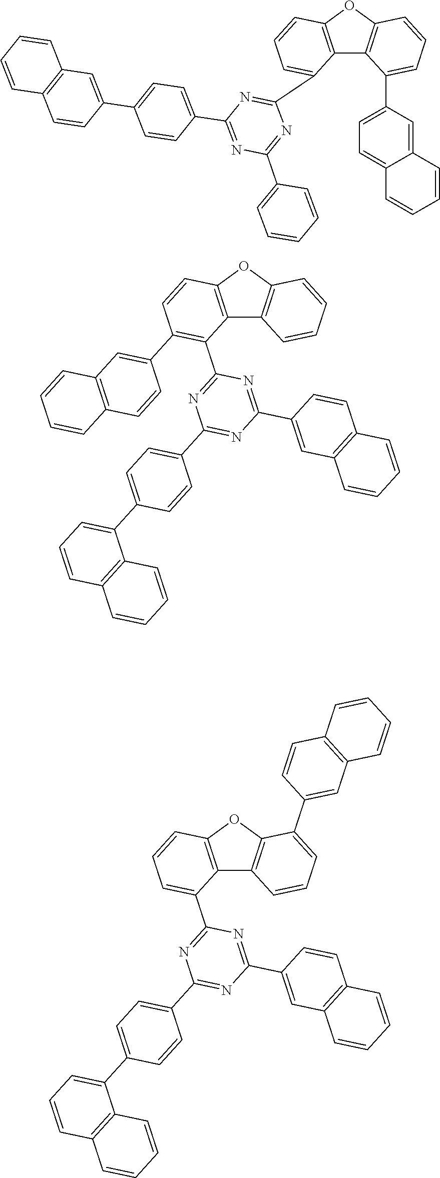

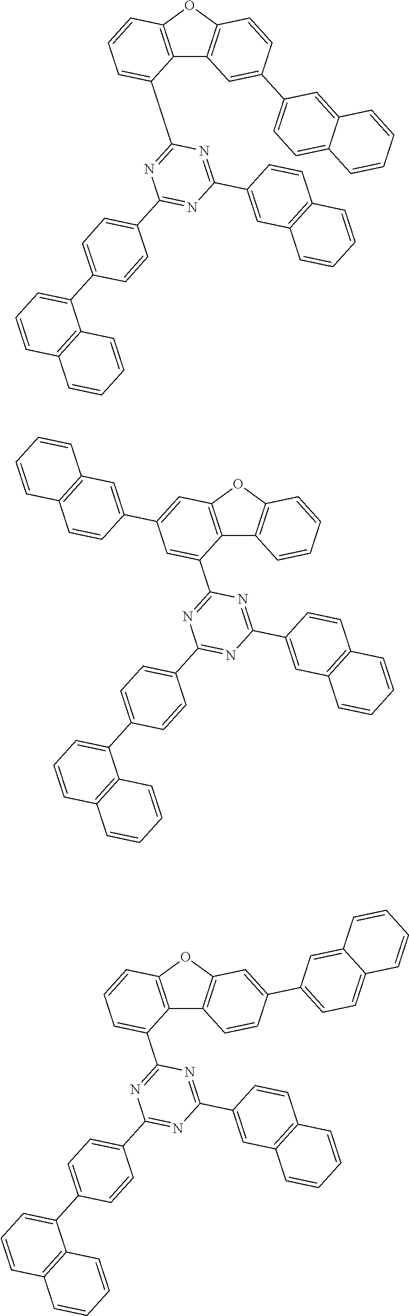 [SMILES: c1ccc2cc(-c3ccc4oc5cccc(-c6nc(-c7ccc(-c8cccc9ccccc89)cc7)nc(-c7ccc8ccccc8c7)n6)c5c4c3)ccc2c1 ; c1ccc2cc(-c3cc(-c4nc(-c5ccc(-c6cccc7ccccc67)cc5)nc(-c5ccc6ccccc6c5)n4)c4c(c3)oc3ccccc34)ccc2c1 ; c1ccc2cc(-c3ccc4c(c3)oc3cccc(-c5nc(-c6ccc(-c7cccc8ccccc78)cc6)nc(-c6ccc7ccccc7c6)n5)c34)ccc2c1]

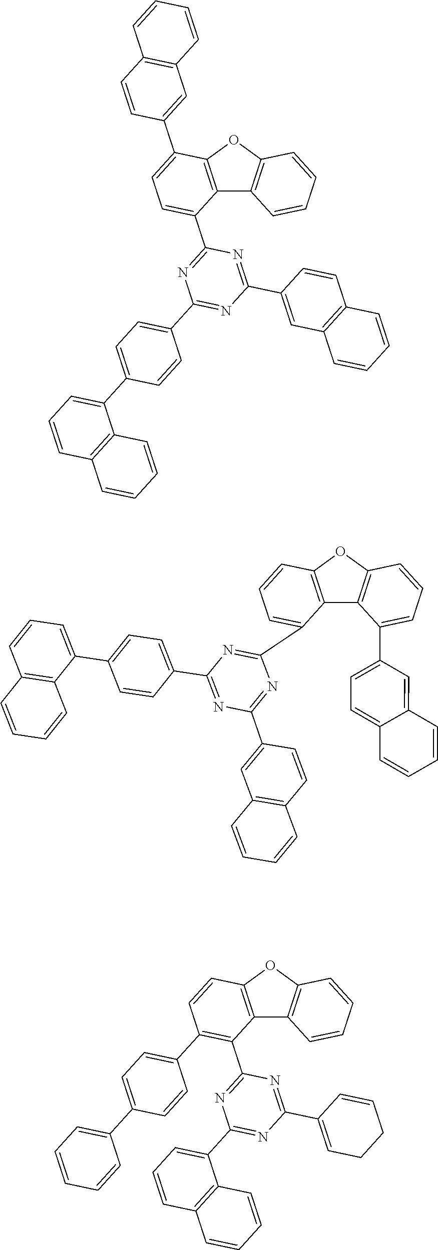 [SMILES: c1ccc2cc(-c3nc(-c4ccc(-c5cccc6ccccc56)cc4)nc(-c4ccc(-c5ccc6ccccc6c5)c5oc6ccccc6c45)n3)ccc2c1 ; c1ccc2cc(-c3nc(-c4ccc(-c5cccc6ccccc56)cc4)nc(-c4cccc5oc6cccc(-c7ccc8ccccc8c7)c6c45)n3)ccc2c1 ; C1=CC(c2nc(-c3cccc4ccccc34)nc(-c3c(-c4ccc(-c5ccccc5)cc4)ccc4oc5ccccc5c34)n2)=CCC1]

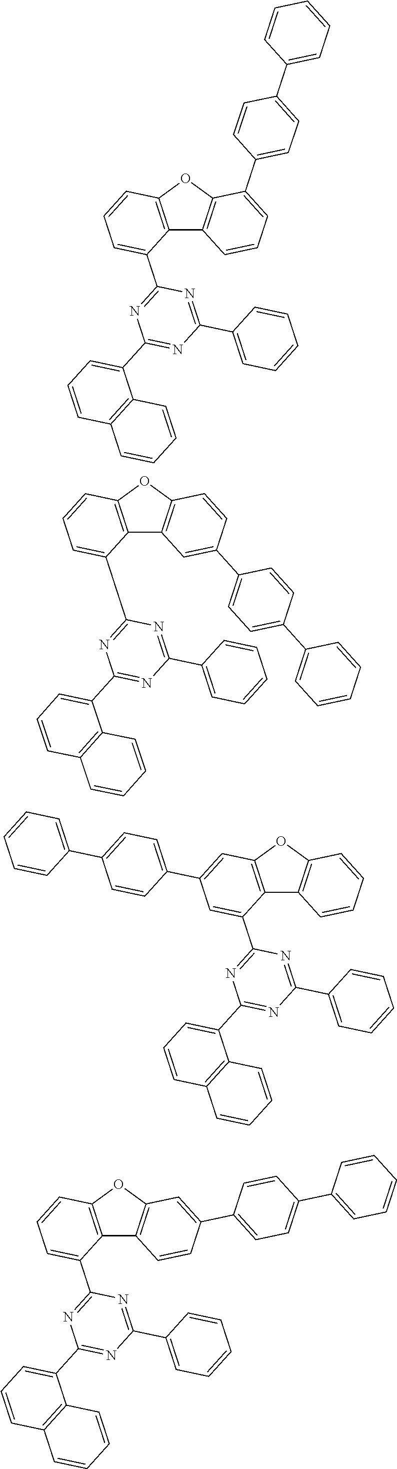

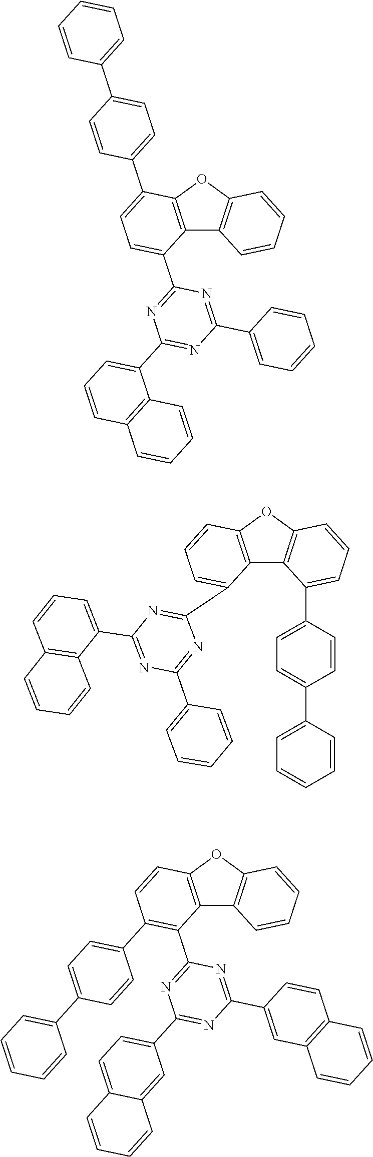 [SMILES: c1ccc(-c2ccc(-c3ccc(-c4nc(-c5ccccc5)nc(-c5cccc6ccccc56)n4)c4c3oc3ccccc34)cc2)cc1 ; c1ccc(-c2ccc(-c3cccc4oc5cccc(-c6nc(-c7ccccc7)nc(-c7cccc8ccccc78)n6)c5c34)cc2)cc1 ; c1ccc(-c2ccc(-c3ccc4oc5ccccc5c4c3-c3nc(-c4ccc5ccccc5c4)nc(-c4ccc5ccccc5c4)n3)cc2)cc1]

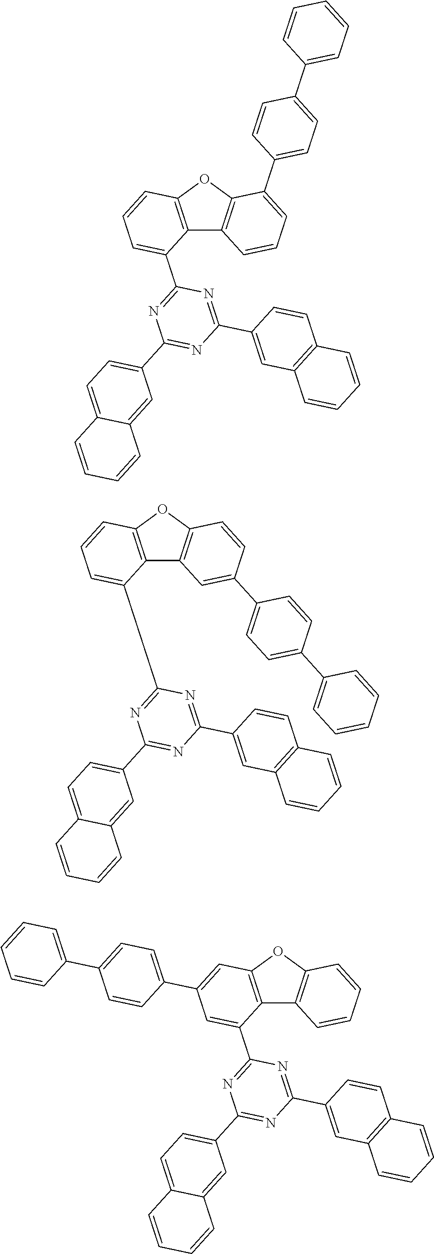

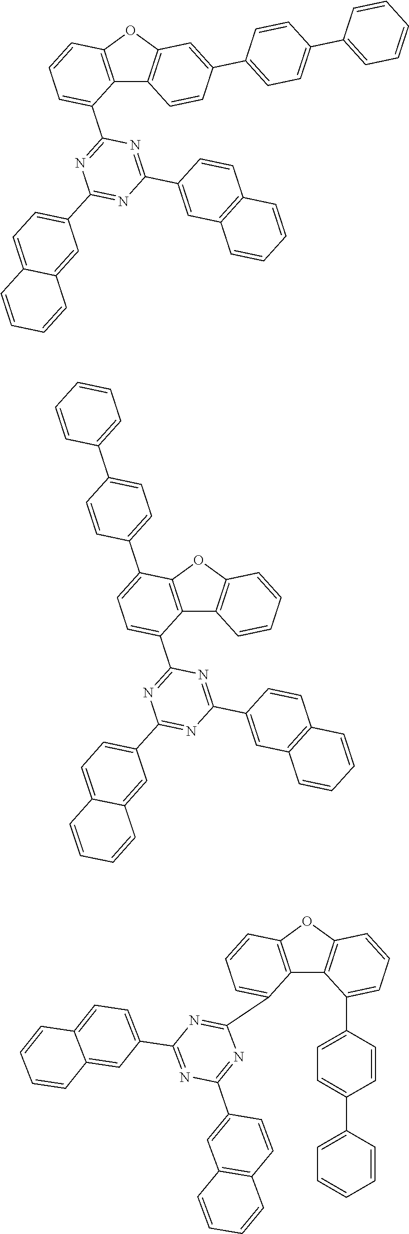 [SMILES: c1ccc(-c2ccc(-c3ccc4c(c3)oc3cccc(-c5nc(-c6ccc7ccccc7c6)nc(-c6ccc7ccccc7c6)n5)c34)cc2)cc1 ; c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1]

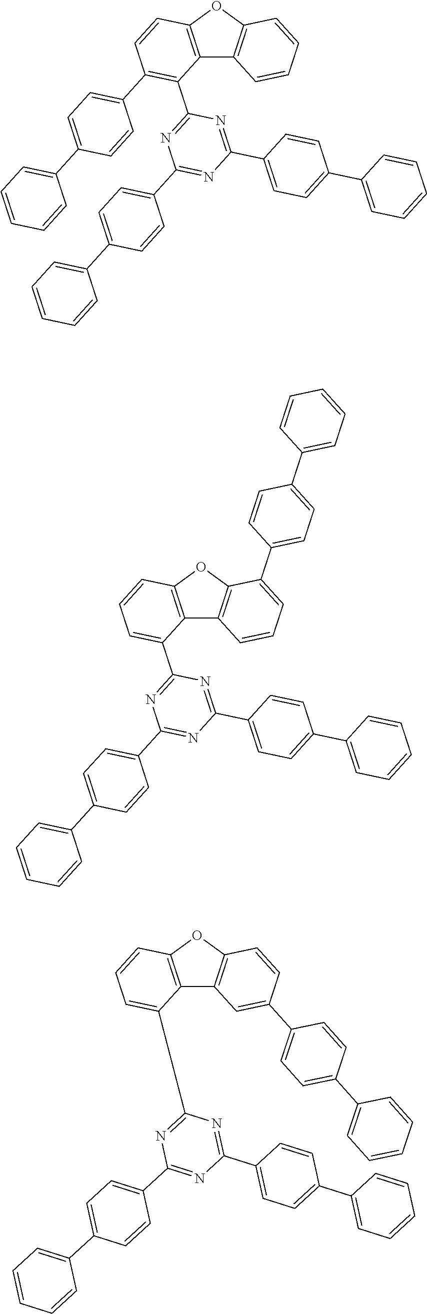 [SMILES: c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccc(-c8ccccc8)cc7)nc(-c7ccc(-c8ccccc8)cc7)n6)c5c4c3)cc2)cc1]

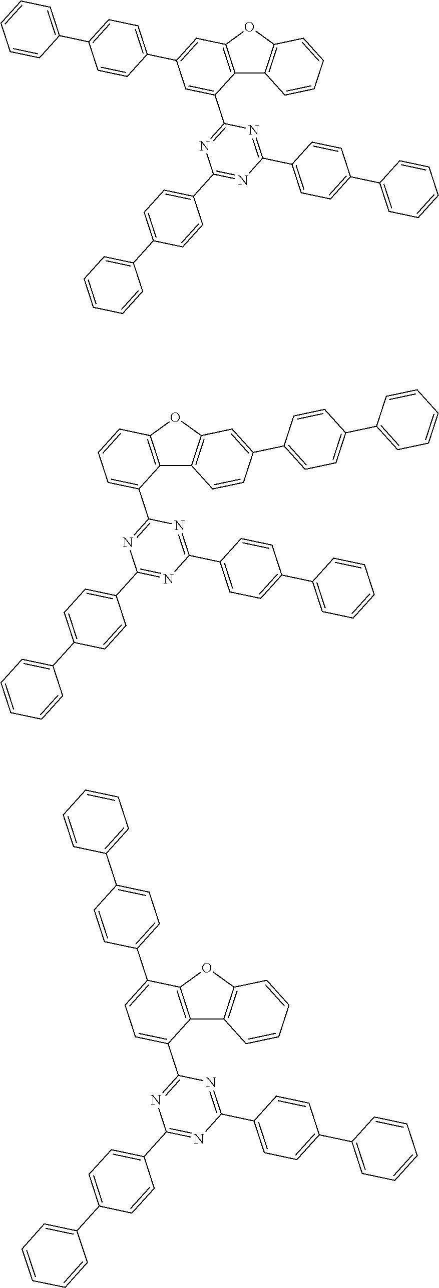 [SMILES: c1ccc(-c2ccc(-c3cc(-c4nc(-c5ccc(-c6ccccc6)cc5)nc(-c5ccc(-c6ccccc6)cc5)n4)c4c(c3)oc3ccccc34)cc2)cc1 ; c1ccc(-c2ccc(-c3ccc4c(c3)oc3cccc(-c5nc(-c6ccc(-c7ccccc7)cc6)nc(-c6ccc(-c7ccccc7)cc6)n5)c34)cc2)cc1 ; c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1]

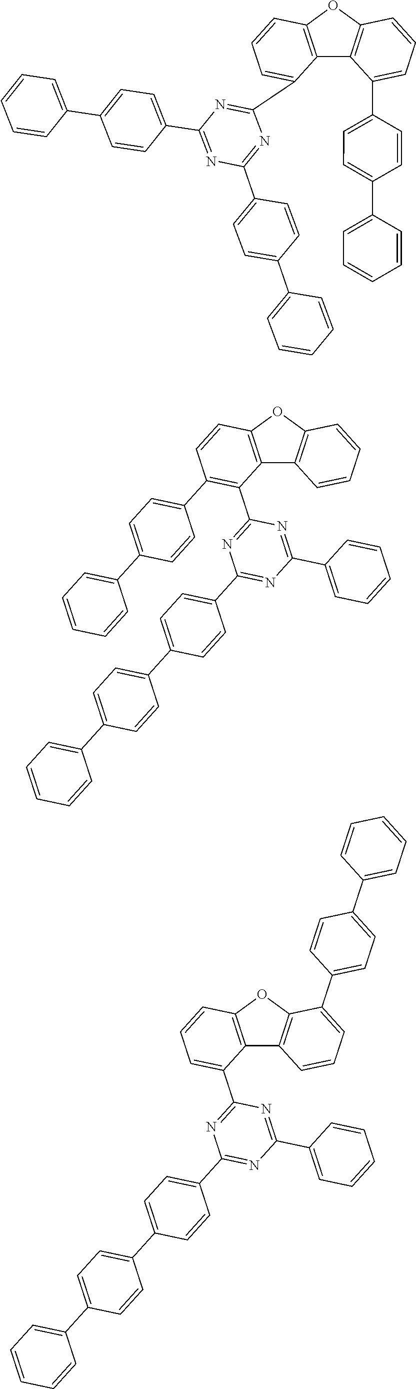 [SMILES: c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1]

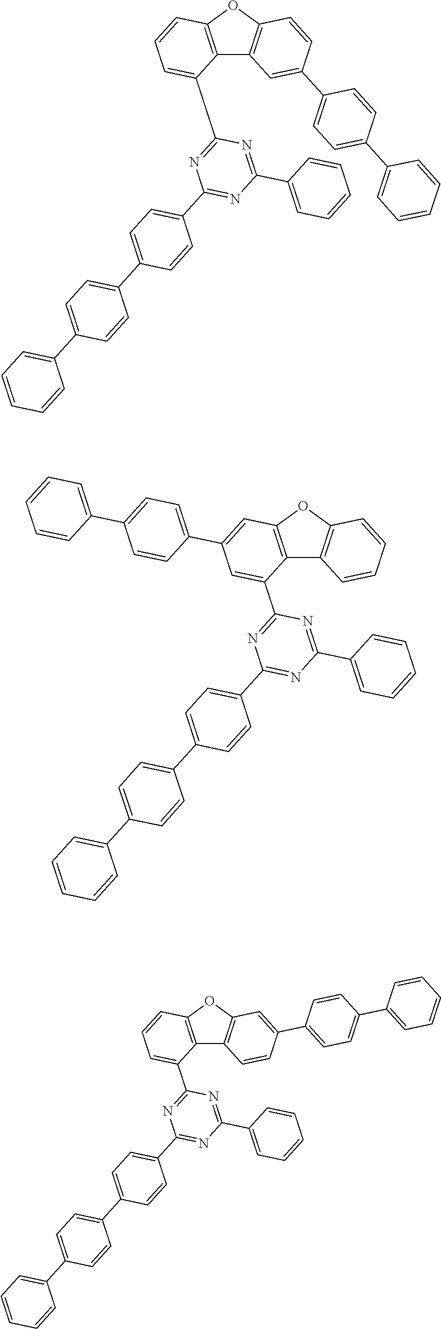 [SMILES: c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1 ; c1ccc(-c2ccc(-c3ccc(-c4nc(-c5ccccc5)nc(-c5cc(-c6ccc(-c7ccccc7)cc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc(-c9ccccc9)cc8)ccc7c56)n4)cc3)cc2)cc1]

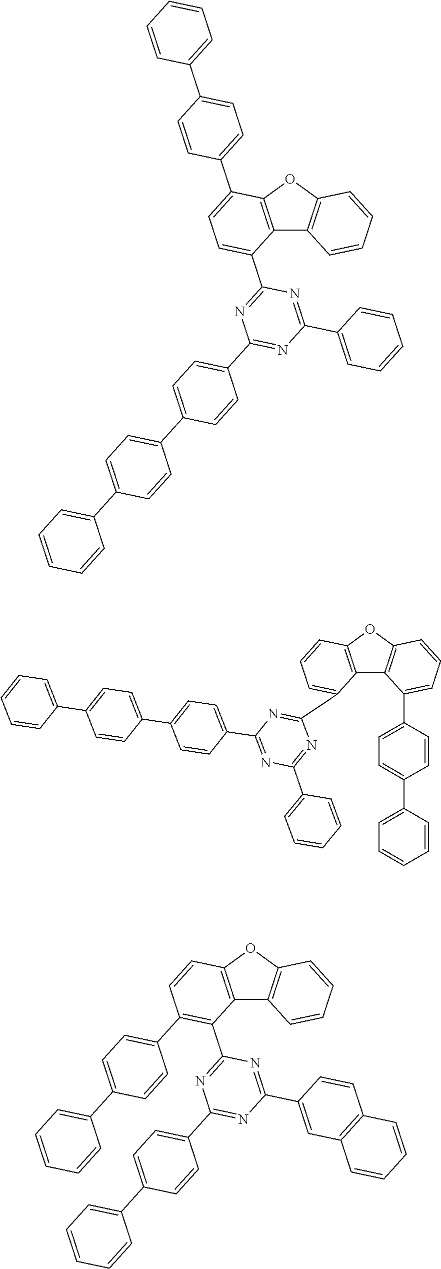 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1 ; c1ccc(-c2ccc(-c3nc(-c4ccc5ccccc5c4)nc(-c4c(-c5ccc(-c6ccccc6)cc5)ccc5oc6ccccc6c45)n3)cc2)cc1]

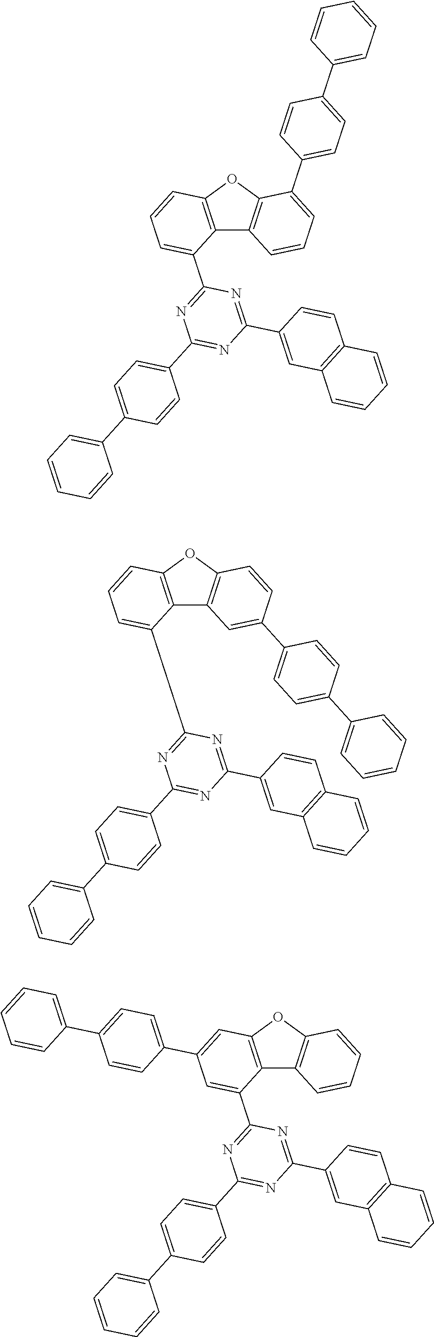 [SMILES: c1ccc(-c2ccc(-c3nc(-c4ccc5ccccc5c4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccc(-c8ccccc8)cc7)nc(-c7ccc8ccccc8c7)n6)c5c4c3)cc2)cc1 ; c1ccc(-c2ccc(-c3cc(-c4nc(-c5ccc(-c6ccccc6)cc5)nc(-c5ccc6ccccc6c5)n4)c4c(c3)oc3ccccc34)cc2)cc1]

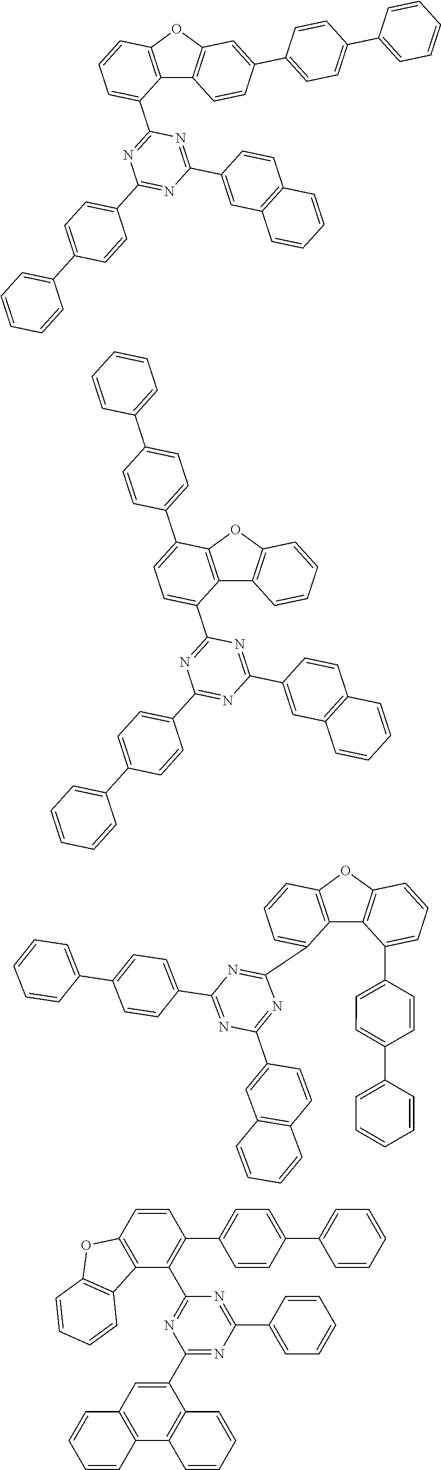

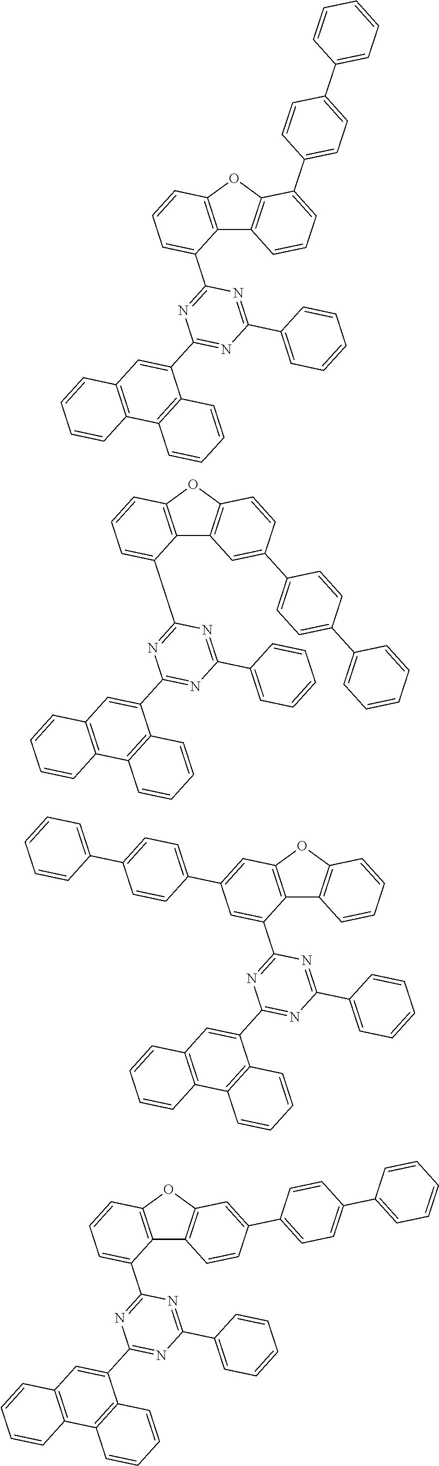

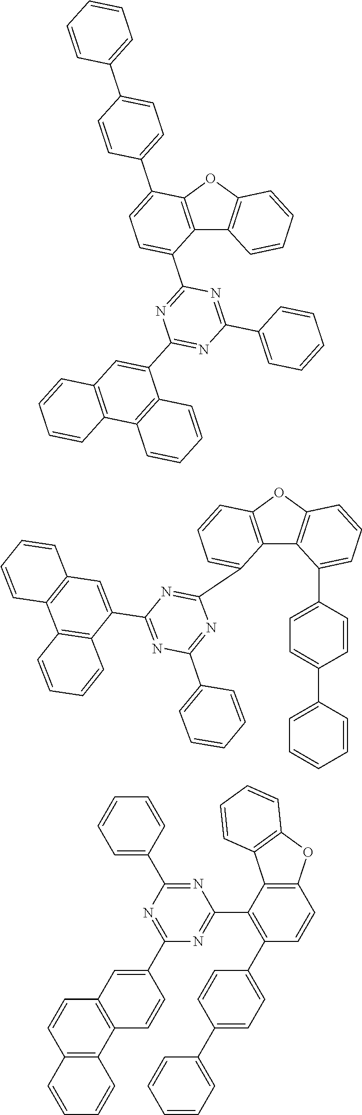

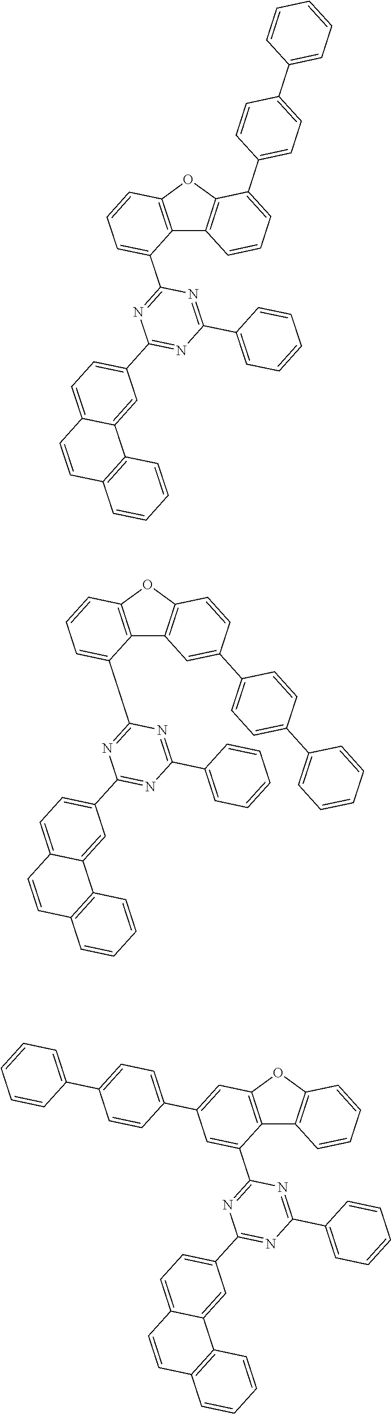 [SMILES: c1ccc(-c2ccc(-c3cccc4c3oc3cccc(-c5nc(-c6ccccc6)nc(-c6ccc7ccc8ccccc8c7c6)n5)c34)cc2)cc1 ; c1ccc(-c2ccc(-c3ccc4oc5cccc(-c6nc(-c7ccccc7)nc(-c7ccc8ccc9ccccc9c8c7)n6)c5c4c3)cc2)cc1 ; c1ccc(-c2ccc(-c3cc(-c4nc(-c5ccccc5)nc(-c5ccc6ccc7ccccc7c6c5)n4)c4c(c3)oc3ccccc34)cc2)cc1]

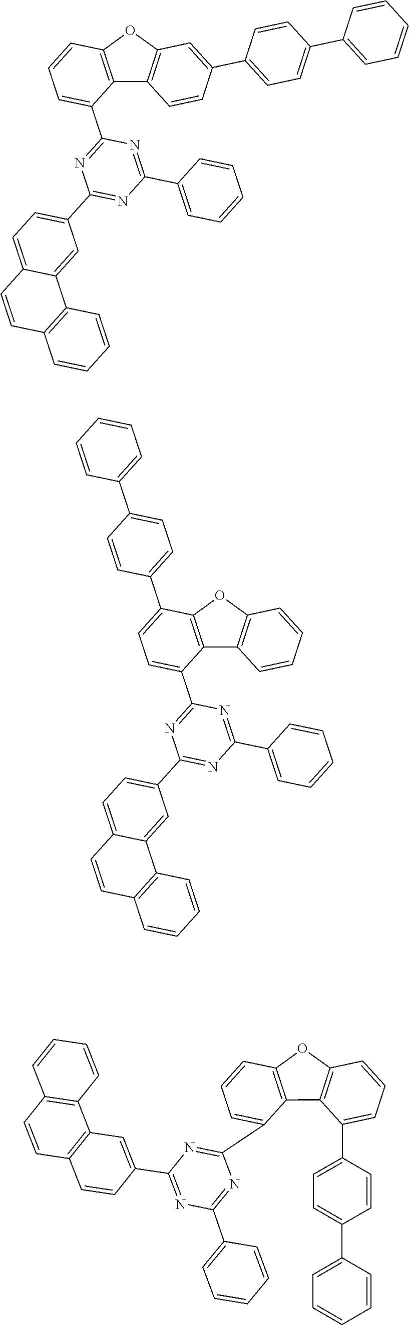 [SMILES: c1ccc(-c2ccc(-c3ccc4c(c3)oc3cccc(-c5nc(-c6ccccc6)nc(-c6ccc7ccc8ccccc8c7c6)n5)c34)cc2)cc1 ; c1ccc(-c2ccc(-c3ccc(-c4nc(-c5ccccc5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccccc7)nc(-c7ccc8ccc9ccccc9c8c7)n6)c5c34)cc2)cc1]

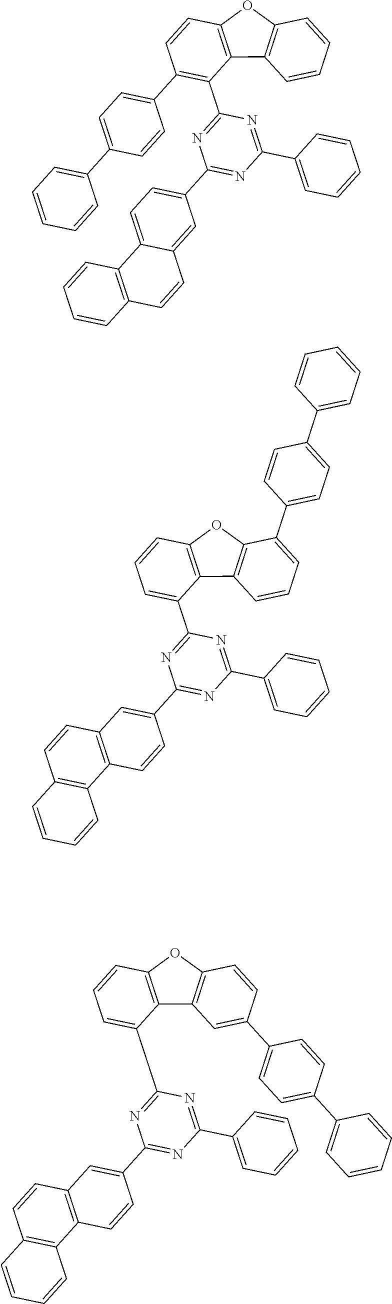 [SMILES: c1ccc(-c2ccc(-c3ccc4oc5ccccc5c4c3-c3nc(-c4ccccc4)nc(-c4ccc5c(ccc6ccccc65)c4)n3)cc2)cc1 ; c1ccc(-c2ccc(-c3cccc4c3oc3cccc(-c5nc(-c6ccccc6)nc(-c6ccc7c(ccc8ccccc87)c6)n5)c34)cc2)cc1 ; c1ccc(-c2ccc(-c3ccc4oc5cccc(-c6nc(-c7ccccc7)nc(-c7ccc8c(ccc9ccccc98)c7)n6)c5c4c3)cc2)cc1]

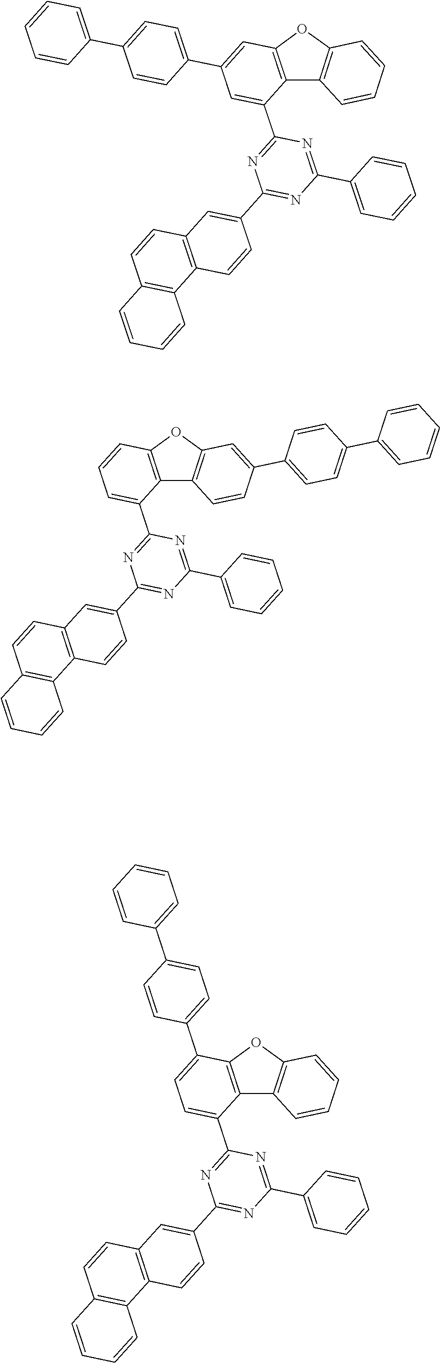 [SMILES: c1ccc(-c2ccc(-c3cc(-c4nc(-c5ccccc5)nc(-c5ccc6c(ccc7ccccc76)c5)n4)c4c(c3)oc3ccccc34)cc2)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2ccc(-c3ccc(-c4nc(-c5ccccc5)nc(-c5ccc6c(ccc7ccccc76)c5)n4)c4c3oc3ccccc34)cc2)cc1]

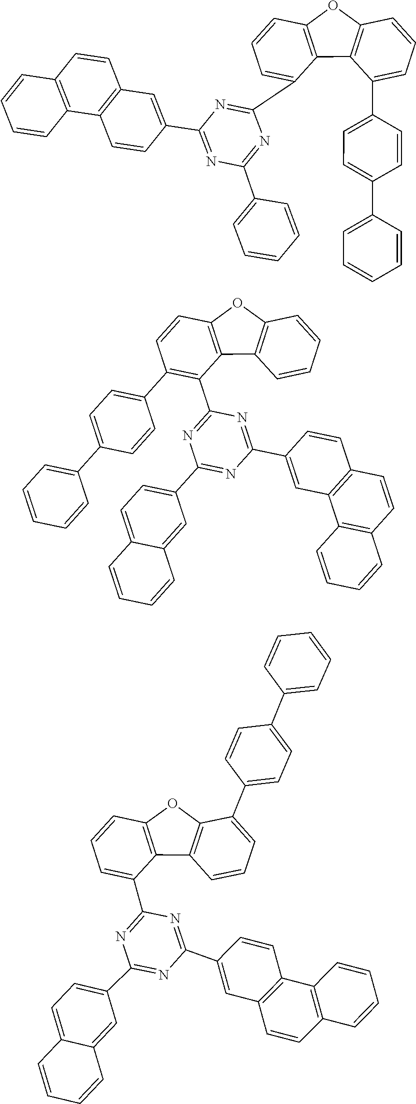 [SMILES: c1ccc(-c2ccc(-c3cccc4oc5cccc(-c6nc(-c7ccccc7)nc(-c7ccc8c(ccc9ccccc98)c7)n6)c5c34)cc2)cc1 ; c1ccc(-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3cccc4c3oc3cccc(-c5nc(-c6ccc7ccccc7c6)nc(-c6ccc7c(ccc8ccccc87)c6)n5)c34)cc2)cc1]

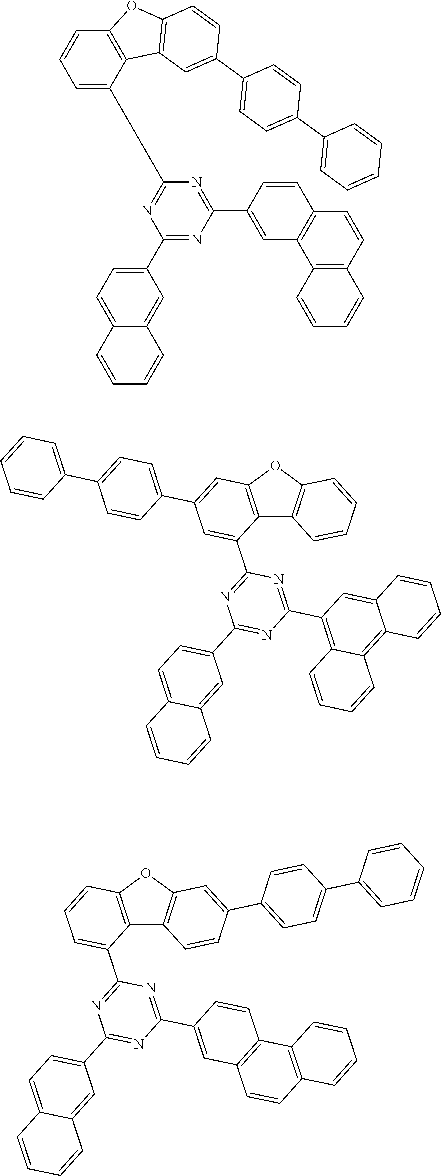 [SMILES: c1ccc(-c2ccc(-c3ccc4oc5cccc(-c6nc(-c7ccc8ccccc8c7)nc(-c7ccc8ccc9ccccc9c8c7)n6)c5c4c3)cc2)cc1 ; c1ccc(-c2ccc(-c3cc(-c4nc(-c5ccc6ccccc6c5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1]

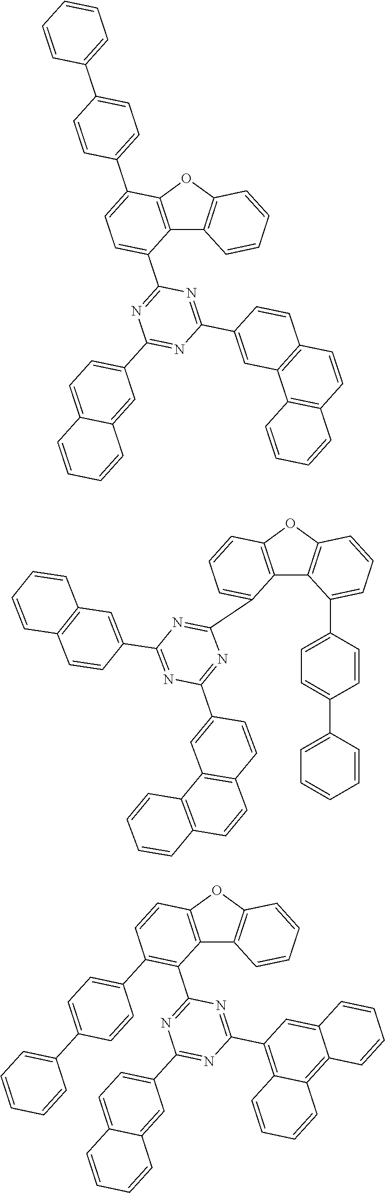 [SMILES: c1ccc(-c2ccc(-c3ccc(-c4nc(-c5ccc6ccccc6c5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccc9ccccc9c8c7)n6)c5c34)cc2)cc1 ; c1ccc(-c2ccc(-c3ccc4oc5ccccc5c4c3-c3nc(-c4ccc5ccccc5c4)nc(-c4cc5ccccc5c5ccccc45)n3)cc2)cc1]

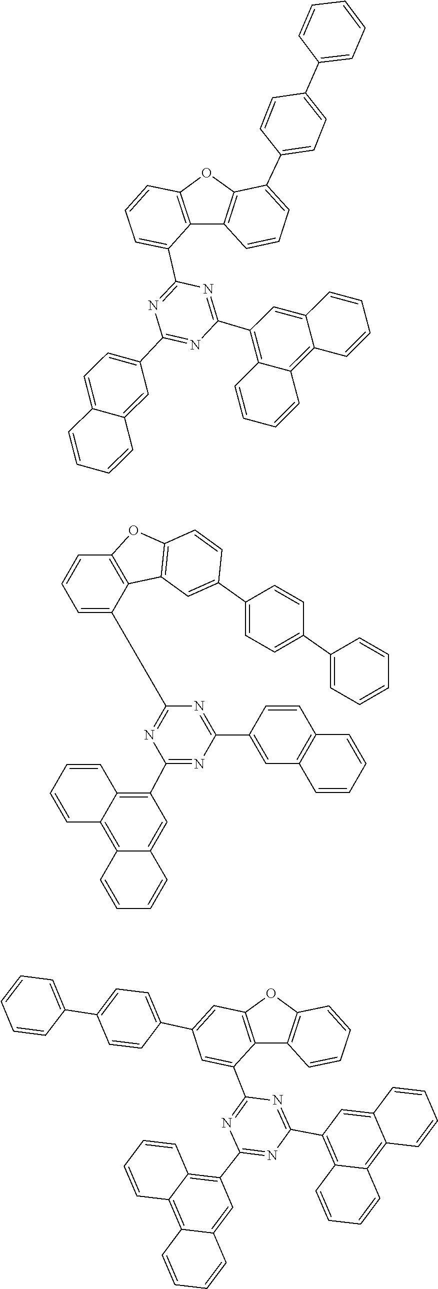 [SMILES: c1ccc(-c2ccc(-c3cccc4c3oc3cccc(-c5nc(-c6ccc7ccccc7c6)nc(-c6cc7ccccc7c7ccccc67)n5)c34)cc2)cc1 ; c1ccc(-c2ccc(-c3ccc4oc5cccc(-c6nc(-c7ccc8ccccc8c7)nc(-c7cc8ccccc8c8ccccc78)n6)c5c4c3)cc2)cc1 ; c1ccc(-c2ccc(-c3cc(-c4nc(-c5cc6ccccc6c6ccccc56)nc(-c5cc6ccccc6c6ccccc56)n4)c4c(c3)oc3ccccc34)cc2)cc1]

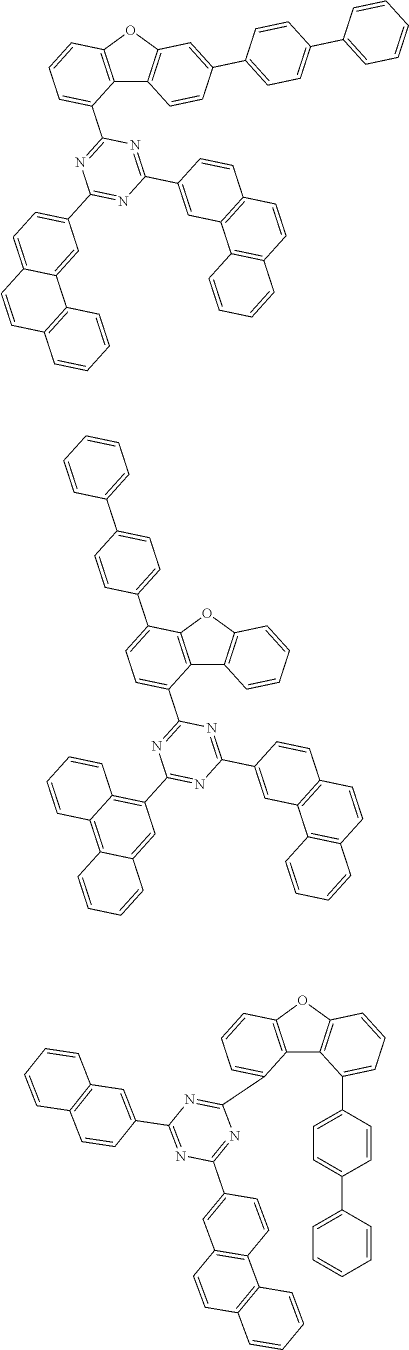 [SMILES: c1ccc(-c2ccc(-c3ccc4c(c3)oc3cccc(-c5nc(-c6ccc7ccc8ccccc8c7c6)nc(-c6ccc7ccc8ccccc8c7c6)n5)c34)cc2)cc1 ; c1ccc(-c2ccc(-c3ccc(-c4nc(-c5ccc6ccc7ccccc7c6c5)nc(-c5cc6ccccc6c6ccccc56)n4)c4c3oc3ccccc34)cc2)cc1 ; c1ccc(-c2ccc(-c3cccc4oc5cccc(-c6nc(-c7ccc8ccccc8c7)nc(-c7ccc8c(ccc9ccccc98)c7)n6)c5c34)cc2)cc1]

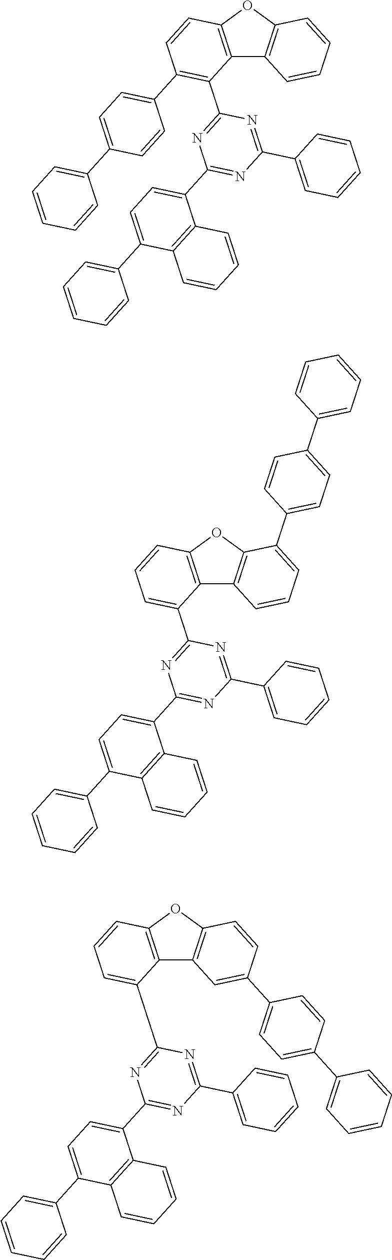 [SMILES: c1ccc(-c2ccc(-c3ccc4oc5ccccc5c4c3-c3nc(-c4ccccc4)nc(-c4ccc(-c5ccccc5)c5ccccc45)n3)cc2)cc1 ; c1ccc(-c2ccc(-c3cccc4c3oc3cccc(-c5nc(-c6ccccc6)nc(-c6ccc(-c7ccccc7)c7ccccc67)n5)c34)cc2)cc1 ; c1ccc(-c2ccc(-c3ccc4oc5cccc(-c6nc(-c7ccccc7)nc(-c7ccc(-c8ccccc8)c8ccccc78)n6)c5c4c3)cc2)cc1]

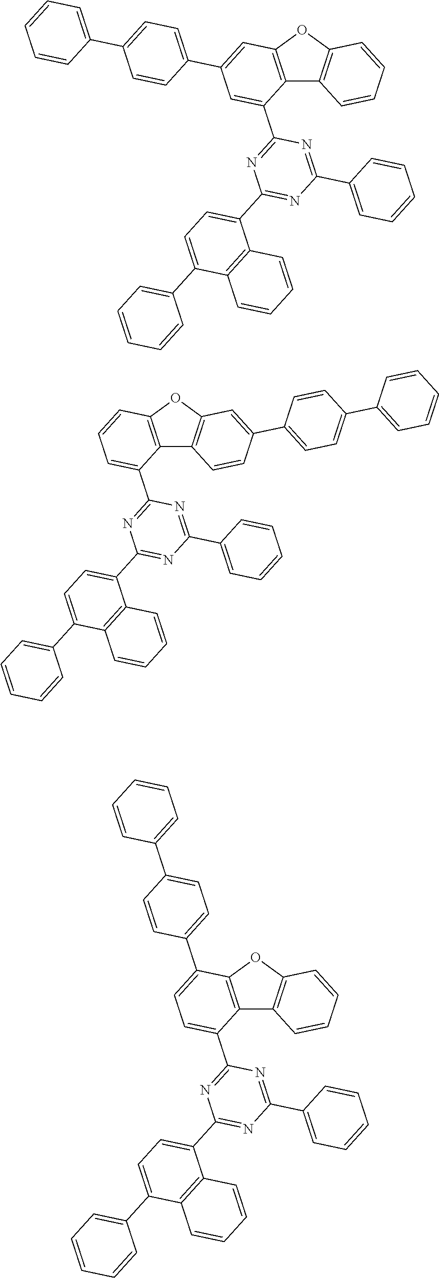 [SMILES: c1ccc(-c2ccc(-c3cc(-c4nc(-c5ccccc5)nc(-c5ccc(-c6ccccc6)c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7ccccc7)c7ccccc67)n5)c34)cc2)cc1 ; c1ccc(-c2ccc(-c3ccc(-c4nc(-c5ccccc5)nc(-c5ccc(-c6ccccc6)c6ccccc56)n4)c4c3oc3ccccc34)cc2)cc1]

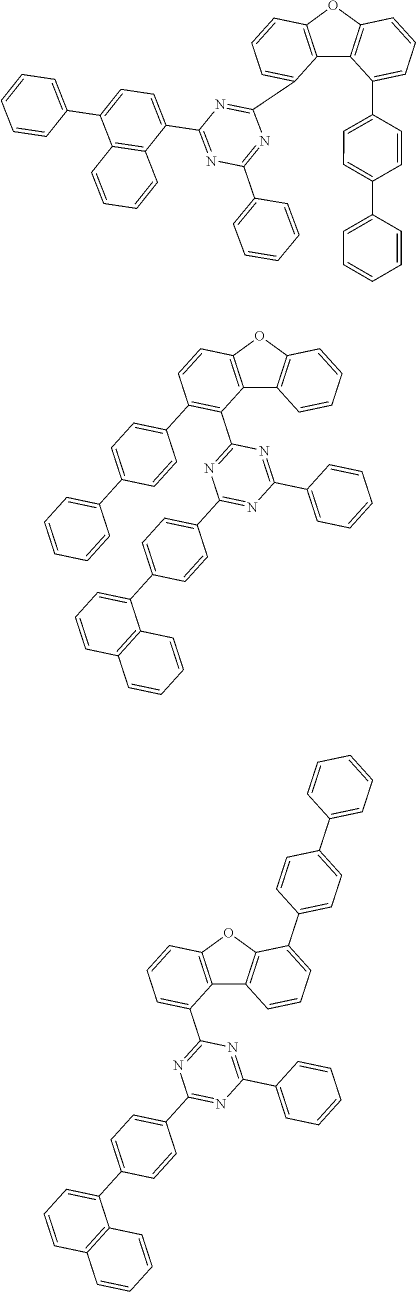 [SMILES: c1ccc(-c2ccc(-c3cccc4oc5cccc(-c6nc(-c7ccccc7)nc(-c7ccc(-c8ccccc8)c8ccccc78)n6)c5c34)cc2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1 ; c1ccc(-c2ccc(-c3cccc4c3oc3cccc(-c5nc(-c6ccccc6)nc(-c6ccc(-c7cccc8ccccc78)cc6)n5)c34)cc2)cc1]

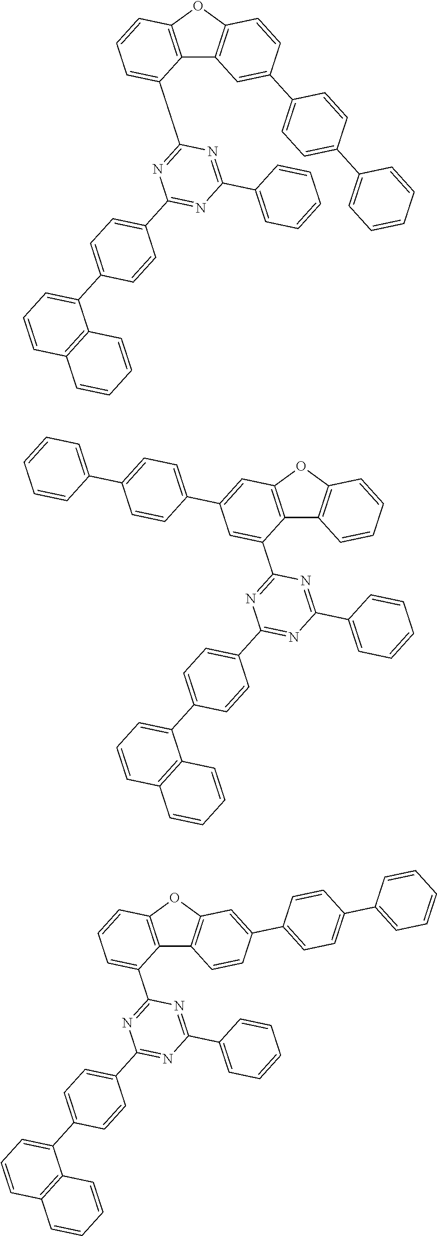 [SMILES: c1ccc(-c2ccc(-c3ccc4oc5cccc(-c6nc(-c7ccccc7)nc(-c7ccc(-c8cccc9ccccc89)cc7)n6)c5c4c3)cc2)cc1 ; c1ccc(-c2ccc(-c3cc(-c4nc(-c5ccccc5)nc(-c5ccc(-c6cccc7ccccc67)cc5)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7cccc8ccccc78)cc6)n5)c34)cc2)cc1]

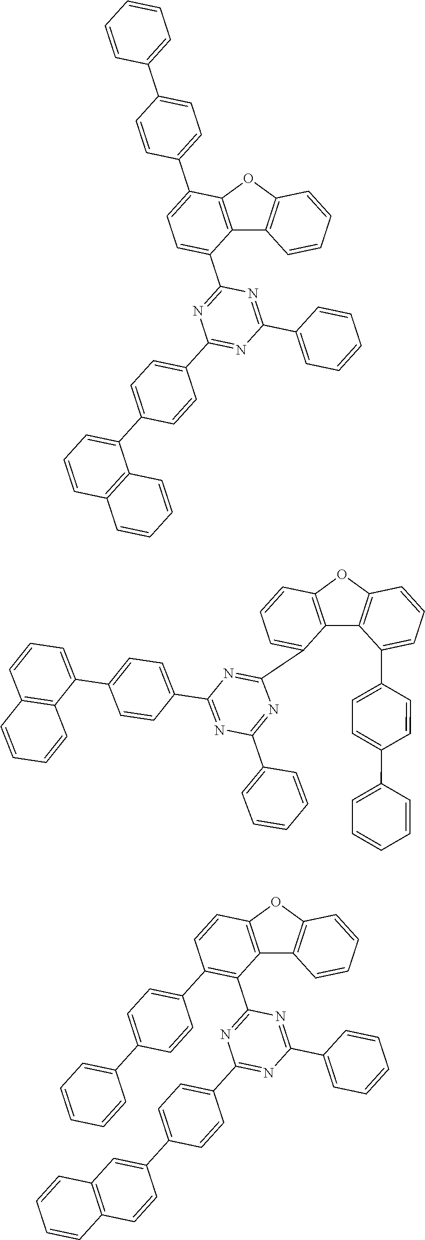 [SMILES: c1ccc(-c2ccc(-c3ccc(-c4nc(-c5ccccc5)nc(-c5ccc(-c6cccc7ccccc67)cc5)n4)c4c3oc3ccccc34)cc2)cc1 ; c1ccc(-c2ccc(-c3cccc4oc5cccc(-c6nc(-c7ccccc7)nc(-c7ccc(-c8cccc9ccccc89)cc7)n6)c5c34)cc2)cc1 ; c1ccc(-c2ccc(-c3ccc4oc5ccccc5c4c3-c3nc(-c4ccccc4)nc(-c4ccc(-c5ccc6ccccc6c5)cc4)n3)cc2)cc1]

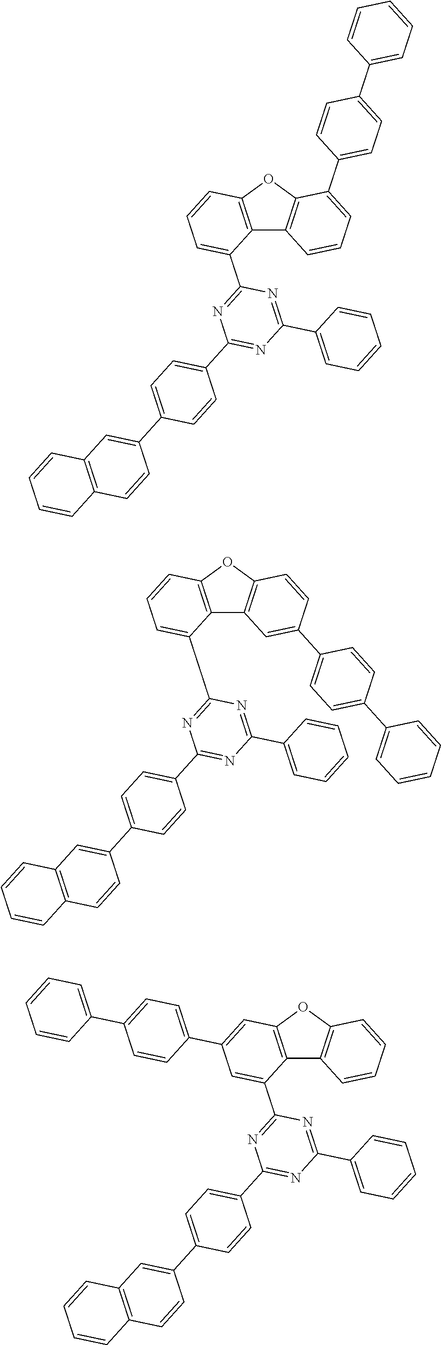 [SMILES: c1ccc(-c2ccc(-c3cccc4c3oc3cccc(-c5nc(-c6ccccc6)nc(-c6ccc(-c7ccc8ccccc8c7)cc6)n5)c34)cc2)cc1 ; c1ccc(-c2ccc(-c3ccc4oc5cccc(-c6nc(-c7ccccc7)nc(-c7ccc(-c8ccc9ccccc9c8)cc7)n6)c5c4c3)cc2)cc1 ; c1ccc(-c2ccc(-c3cc(-c4nc(-c5ccccc5)nc(-c5ccc(-c6ccc7ccccc7c6)cc5)n4)c4c(c3)oc3ccccc34)cc2)cc1]

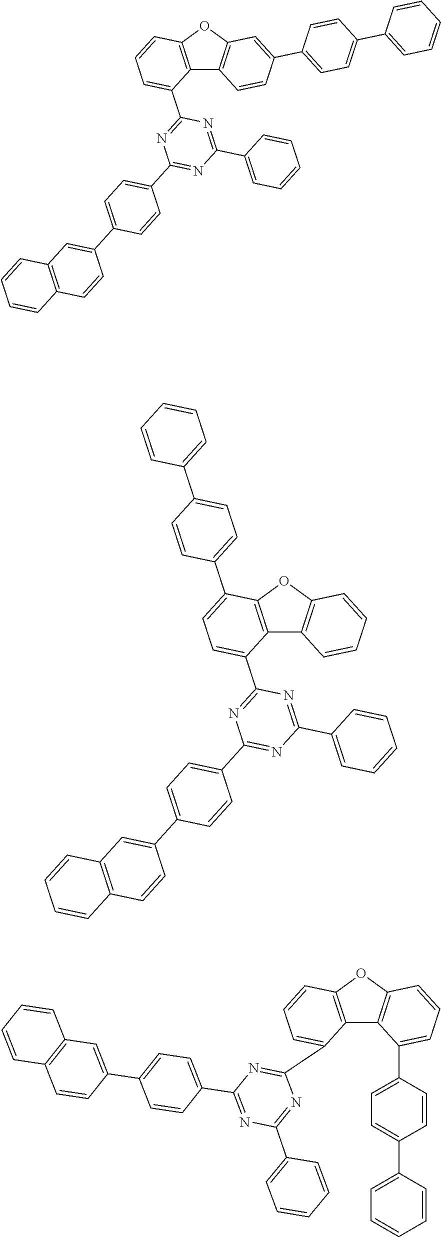 [SMILES: c1ccc(-c2ccc(-c3ccc4c(c3)oc3cccc(-c5nc(-c6ccccc6)nc(-c6ccc(-c7ccc8ccccc8c7)cc6)n5)c34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1 ; c1ccc(-c2ccc(-c3cccc4oc5cccc(-c6nc(-c7ccccc7)nc(-c7ccc(-c8ccc9ccccc9c8)cc7)n6)c5c34)cc2)cc1]

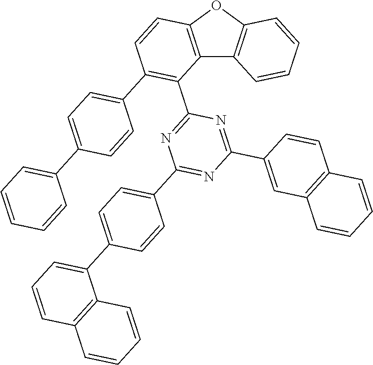 [SMILES: c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

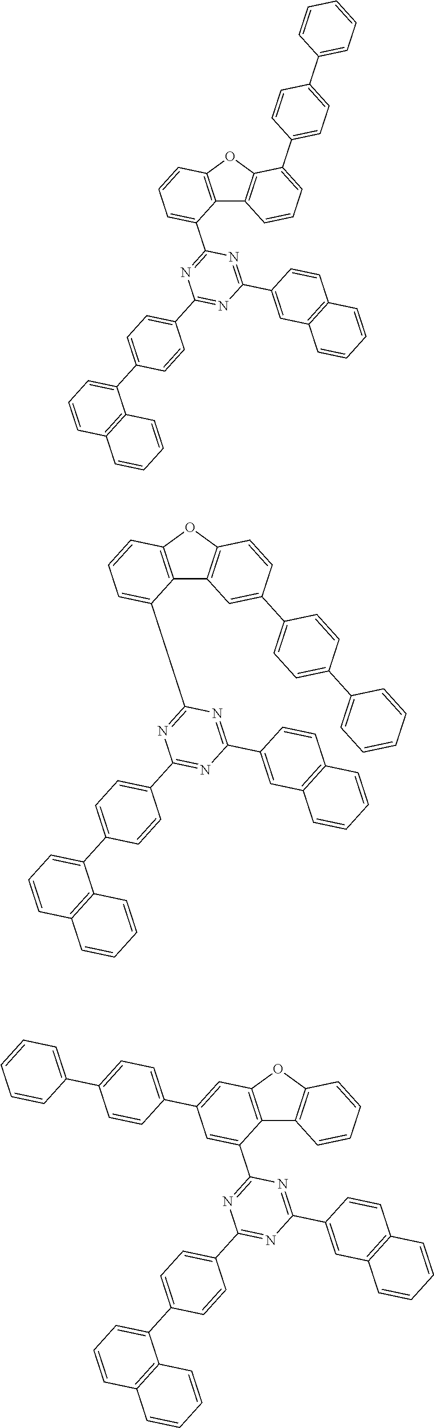 [SMILES: c1ccc(-c2ccc(-c3cccc4c3oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc4oc5cccc(-c6nc(-c7ccc(-c8cccc9ccccc89)cc7)nc(-c7ccc8ccccc8c7)n6)c5c4c3)cc2)cc1 ; c1ccc(-c2ccc(-c3cc(-c4nc(-c5ccc(-c6cccc7ccccc67)cc5)nc(-c5ccc6ccccc6c5)n4)c4c(c3)oc3ccccc34)cc2)cc1]

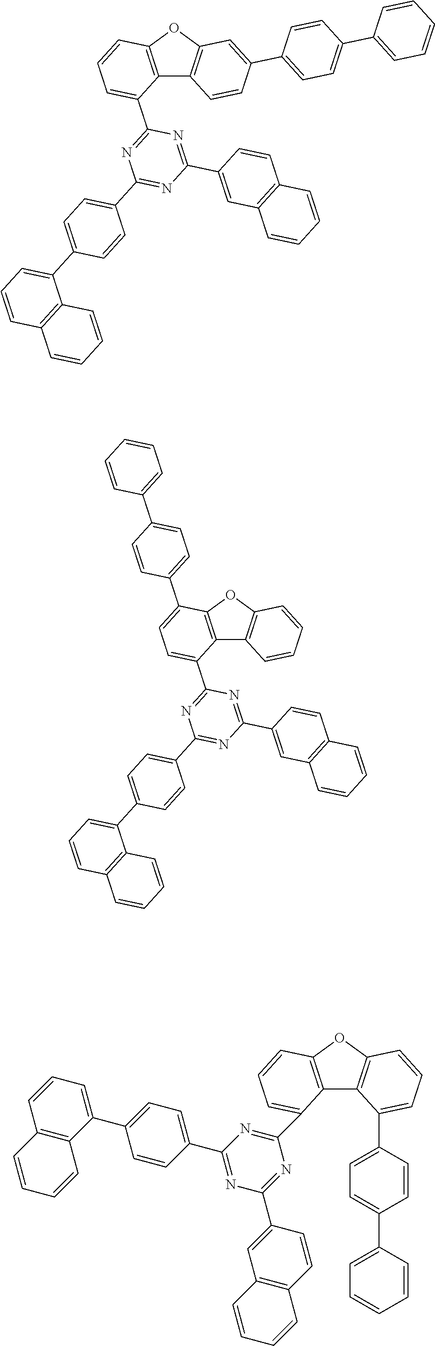 [SMILES: c1ccc(-c2ccc(-c3ccc4c(c3)oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc(-c4nc(-c5ccc(-c6cccc7ccccc67)cc5)nc(-c5ccc6ccccc6c5)n4)c4c3oc3ccccc34)cc2)cc1 ; c1ccc(-c2ccc(-c3cccc4oc5cccc(-c6nc(-c7ccc(-c8cccc9ccccc89)cc7)nc(-c7ccc8ccccc8c7)n6)c5c34)cc2)cc1]

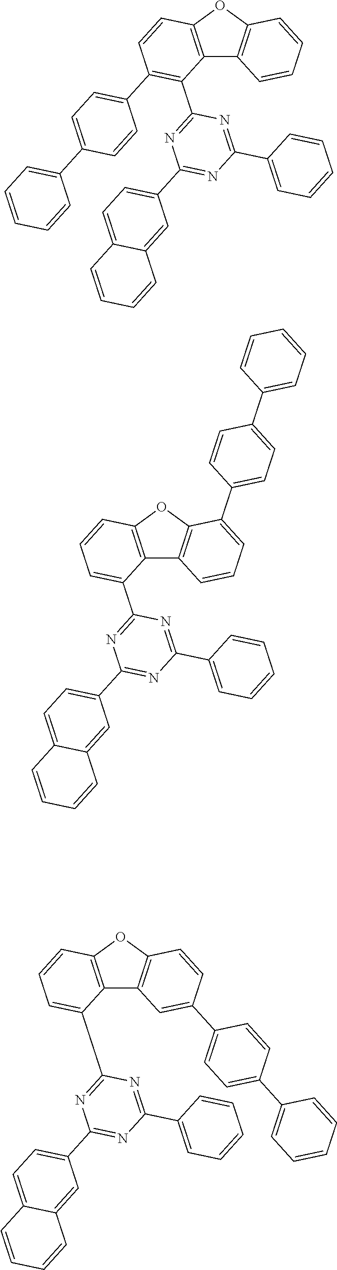 [SMILES: c1ccc(-c2ccc(-c3ccc4oc5ccccc5c4c3-c3nc(-c4ccccc4)nc(-c4ccc5ccccc5c4)n3)cc2)cc1 ; c1ccc(-c2ccc(-c3cccc4c3oc3cccc(-c5nc(-c6ccccc6)nc(-c6ccc7ccccc7c6)n5)c34)cc2)cc1 ; c1ccc(-c2ccc(-c3ccc4oc5cccc(-c6nc(-c7ccccc7)nc(-c7ccc8ccccc8c7)n6)c5c4c3)cc2)cc1]

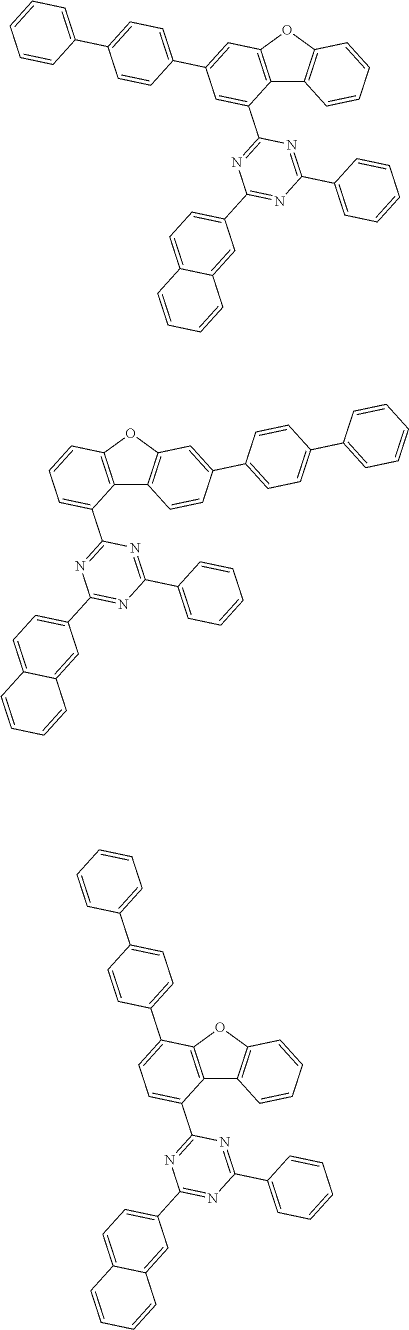 [SMILES: c1ccc(-c2ccc(-c3cc(-c4nc(-c5ccccc5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1]

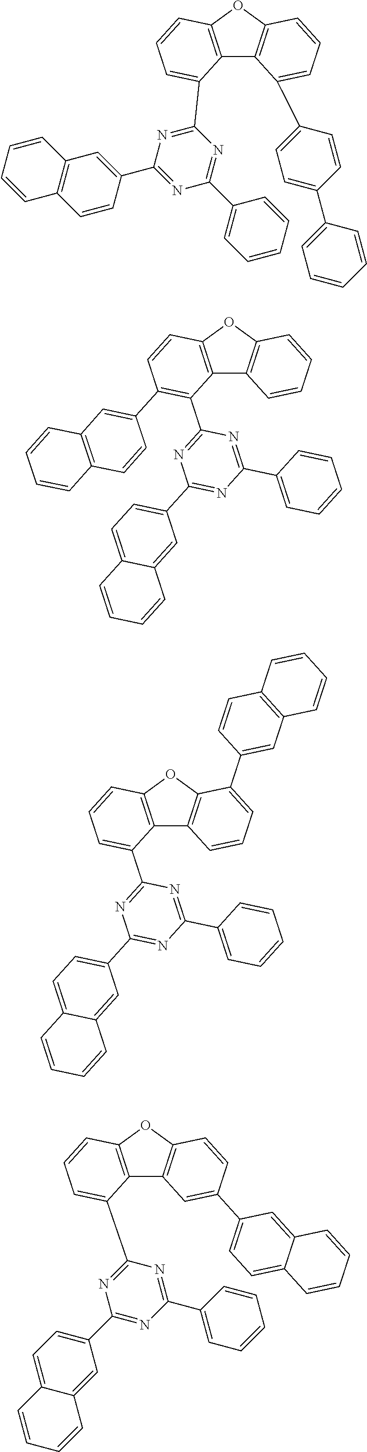 [SMILES: c1ccc(-c2ccc(-c3cccc4oc5cccc(-c6nc(-c7ccccc7)nc(-c7ccc8ccccc8c7)n6)c5c34)cc2)cc1 ; c1ccc(-c2nc(-c3ccc4ccccc4c3)nc(-c3c(-c4ccc5ccccc5c4)ccc4oc5ccccc5c34)n2)cc1 ; c1ccc(-c2nc(-c3ccc4ccccc4c3)nc(-c3cccc4oc5c(-c6ccc7ccccc7c6)cccc5c34)n2)cc1 ; c1ccc(-c2nc(-c3ccc4ccccc4c3)nc(-c3cccc4oc5ccc(-c6ccc7ccccc7c6)cc5c34)n2)cc1]

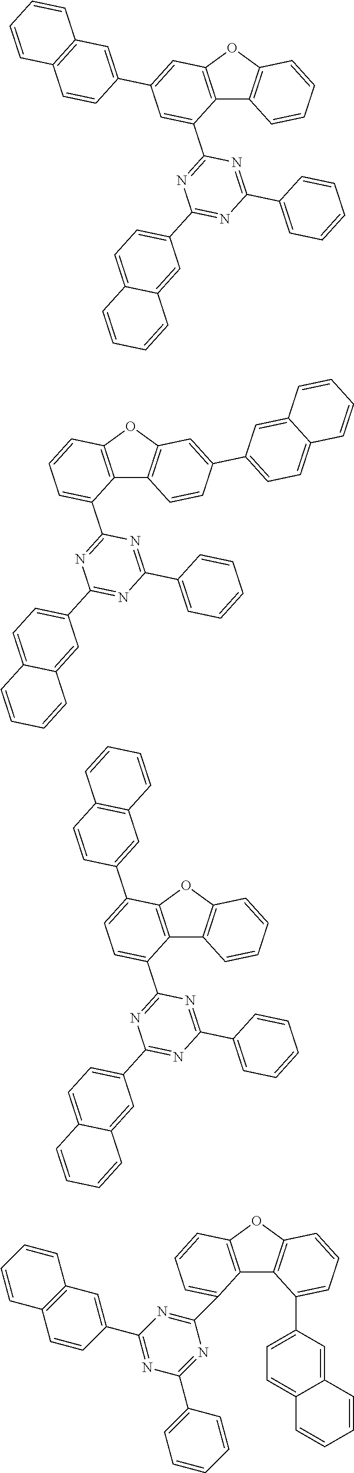 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2nc(-c3ccc4ccccc4c3)nc(-c3ccc(-c4ccc5ccccc5c4)c4oc5ccccc5c34)n2)cc1 ; c1ccc(-c2nc(-c3ccc4ccccc4c3)nc(-c3cccc4oc5cccc(-c6ccc7ccccc7c6)c5c34)n2)cc1]

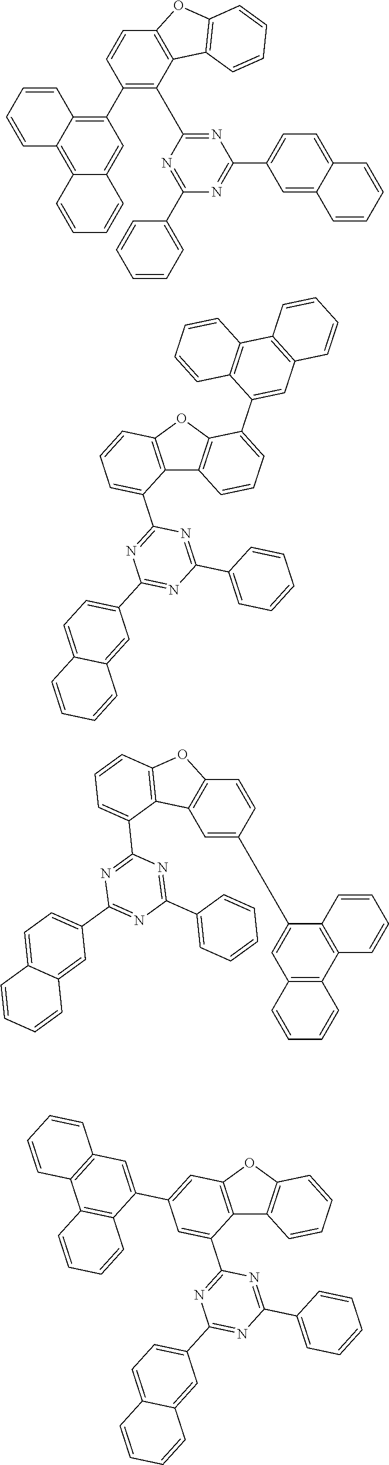

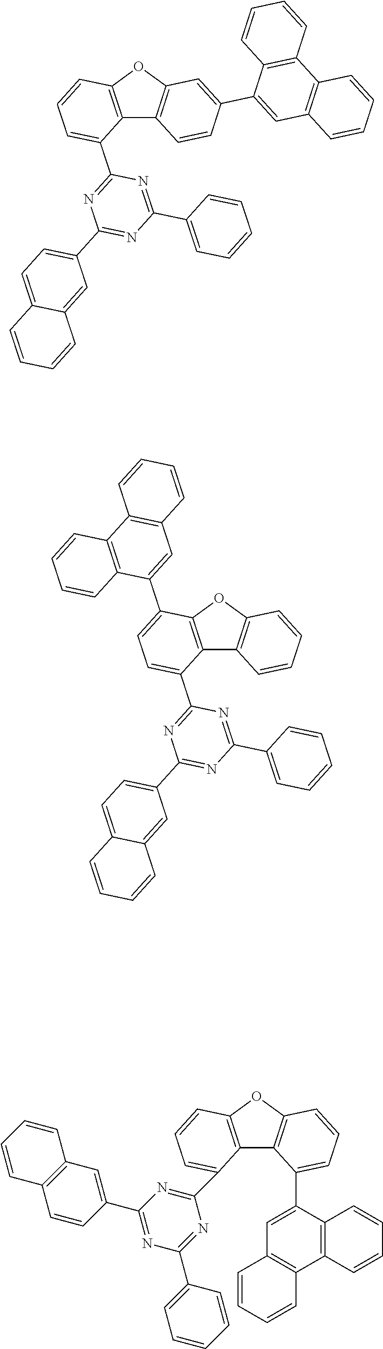 [SMILES: c1ccc(-c2nc(-c3ccc4ccccc4c3)nc(-c3cccc4oc5cc(-c6cc7ccccc7c7ccccc67)ccc5c34)n2)cc1 ; c1ccc(-c2nc(-c3ccc4ccccc4c3)nc(-c3ccc(-c4cc5ccccc5c5ccccc45)c4oc5ccccc5c34)n2)cc1 ; c1ccc(-c2nc(-c3ccc4ccccc4c3)nc(-c3cccc4oc5cccc(-c6cc7ccccc7c7ccccc67)c5c34)n2)cc1]

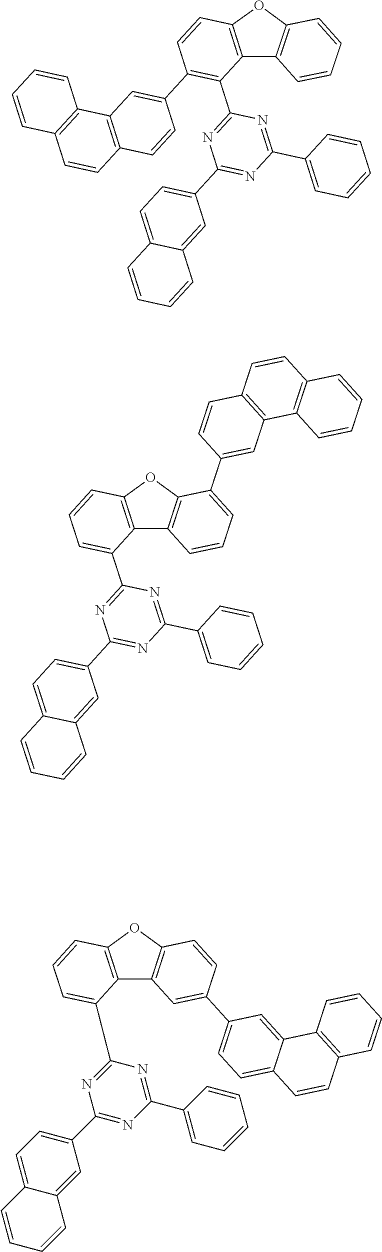 [SMILES: c1ccc(-c2nc(-c3ccc4ccccc4c3)nc(-c3c(-c4ccc5ccc6ccccc6c5c4)ccc4oc5ccccc5c34)n2)cc1 ; c1ccc(-c2nc(-c3ccc4ccccc4c3)nc(-c3cccc4oc5c(-c6ccc7ccc8ccccc8c7c6)cccc5c34)n2)cc1 ; c1ccc(-c2nc(-c3ccc4ccccc4c3)nc(-c3cccc4oc5ccc(-c6ccc7ccc8ccccc8c7c6)cc5c34)n2)cc1]

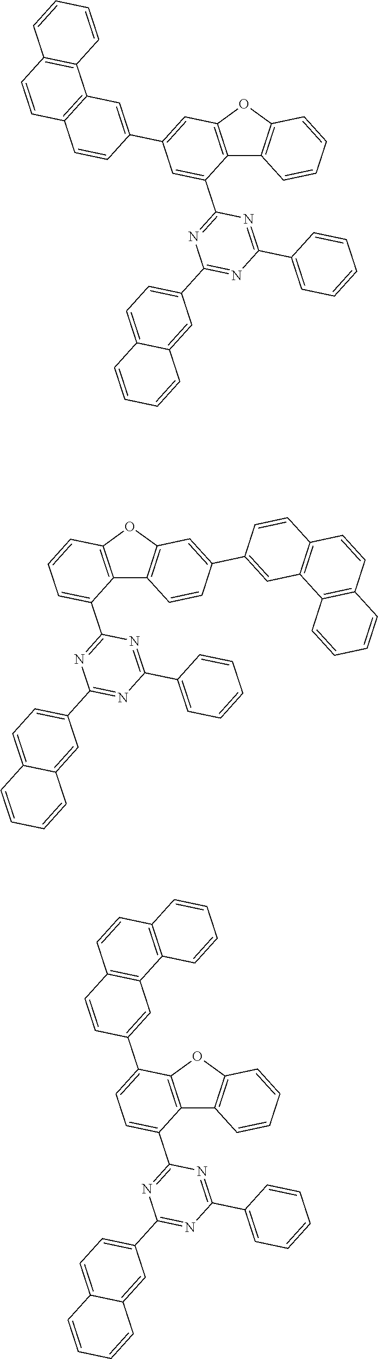 [SMILES: c1ccc(-c2nc(-c3ccc4ccccc4c3)nc(-c3cc(-c4ccc5ccc6ccccc6c5c4)cc4oc5ccccc5c34)n2)cc1 ; c1ccc(-c2nc(-c3ccc4ccccc4c3)nc(-c3cccc4oc5cc(-c6ccc7ccc8ccccc8c7c6)ccc5c34)n2)cc1 ; c1ccc(-c2nc(-c3ccc4ccccc4c3)nc(-c3ccc(-c4ccc5ccc6ccccc6c5c4)c4oc5ccccc5c34)n2)cc1]

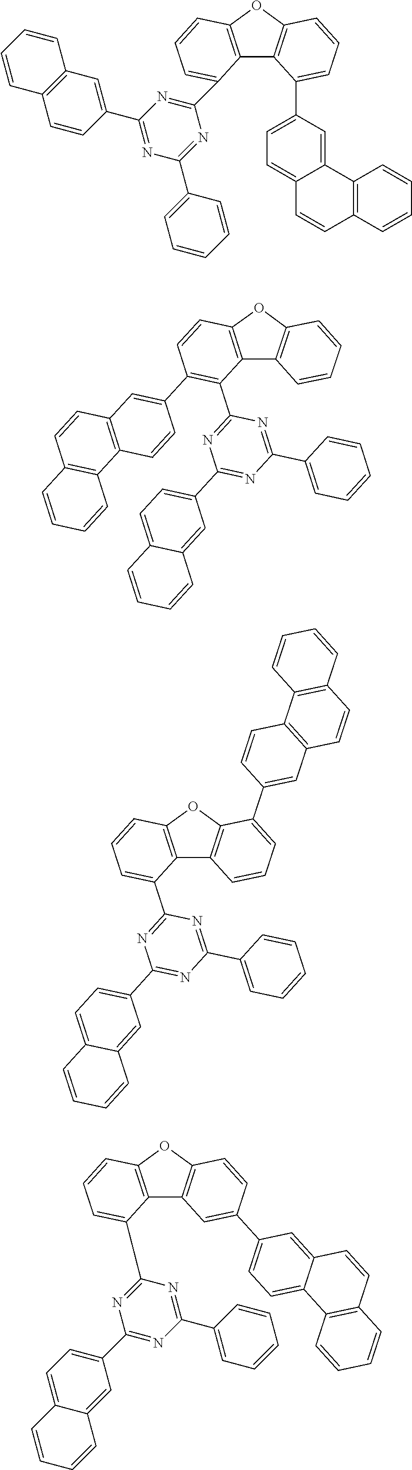 [SMILES: c1ccc(-c2nc(-c3ccc4ccccc4c3)nc(-c3cccc4oc5cccc(-c6ccc7ccc8ccccc8c7c6)c5c34)n2)cc1 ; c1ccc(-c2nc(-c3ccc4ccccc4c3)nc(-c3c(-c4ccc5c(ccc6ccccc65)c4)ccc4oc5ccccc5c34)n2)cc1 ; c1ccc(-c2nc(-c3ccc4ccccc4c3)nc(-c3cccc4oc5c(-c6ccc7c(ccc8ccccc87)c6)cccc5c34)n2)cc1 ; c1ccc(-c2nc(-c3ccc4ccccc4c3)nc(-c3cccc4oc5ccc(-c6ccc7c(ccc8ccccc87)c6)cc5c34)n2)cc1]

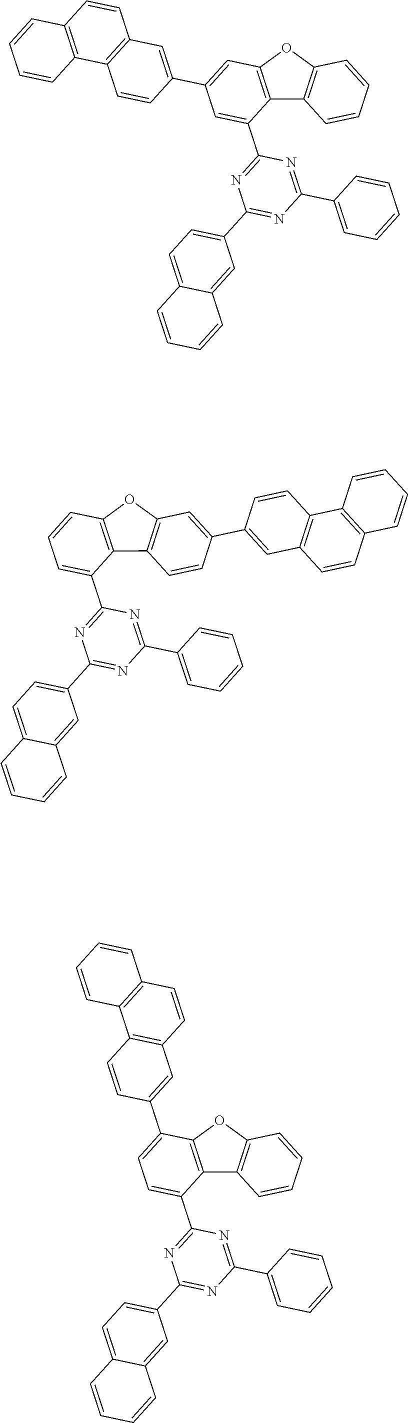 [SMILES: c1ccc(-c2nc(-c3ccc4ccccc4c3)nc(-c3cc(-c4ccc5c(ccc6ccccc65)c4)cc4oc5ccccc5c34)n2)cc1 ; c1ccc(-c2nc(-c3ccc4ccccc4c3)nc(-c3cccc4oc5cc(-c6ccc7c(ccc8ccccc87)c6)ccc5c34)n2)cc1 ; c1ccc(-c2nc(-c3ccc4ccccc4c3)nc(-c3ccc(-c4ccc5c(ccc6ccccc65)c4)c4oc5ccccc5c34)n2)cc1]

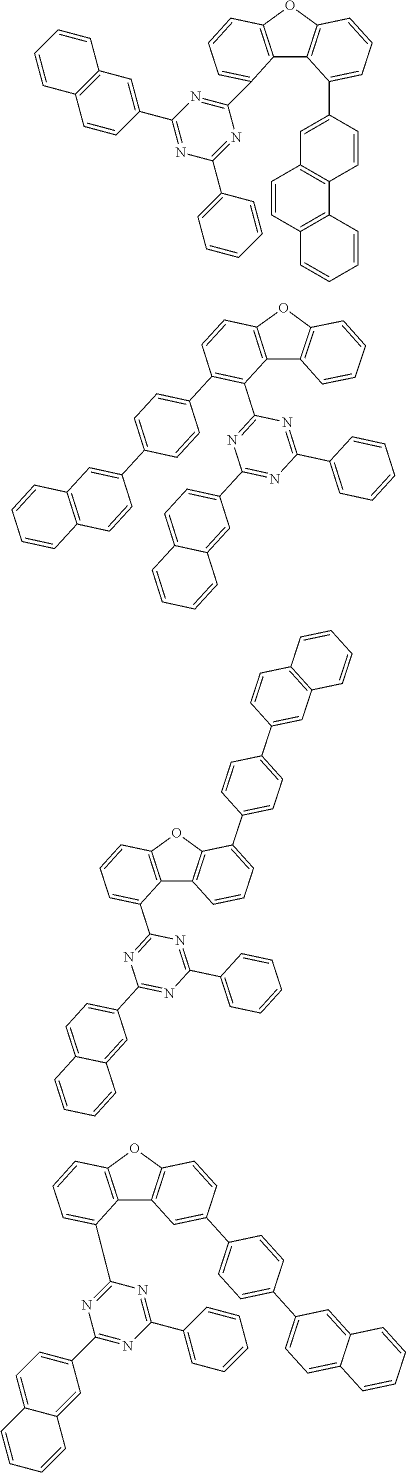

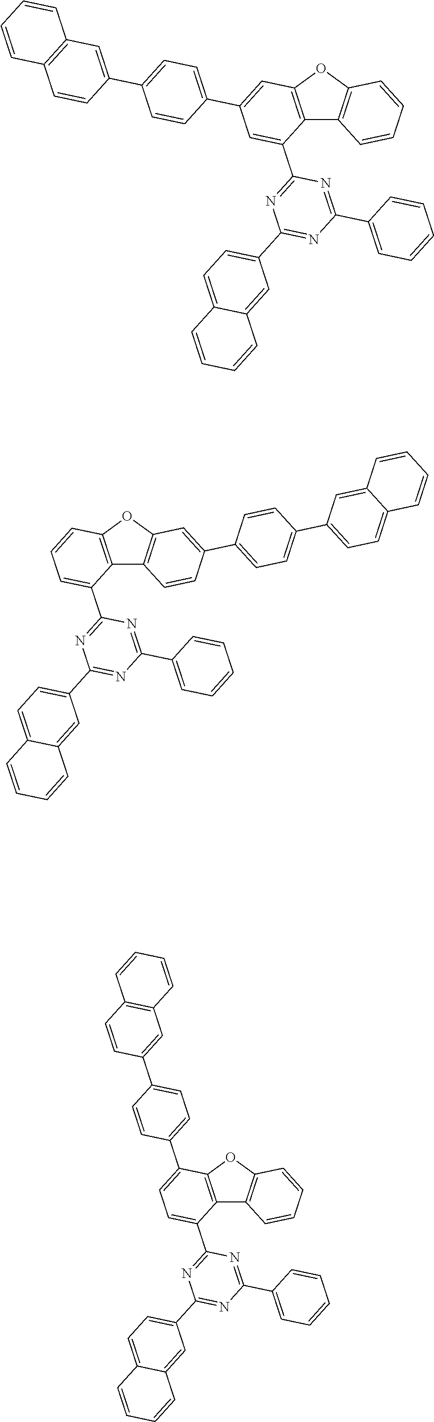 [SMILES: c1ccc(-c2nc(-c3ccc4ccccc4c3)nc(-c3cc(-c4ccc(-c5ccc6ccccc6c5)cc4)cc4oc5ccccc5c34)n2)cc1 ; c1ccc(-c2nc(-c3ccc4ccccc4c3)nc(-c3cccc4oc5cc(-c6ccc(-c7ccc8ccccc8c7)cc6)ccc5c34)n2)cc1 ; c1ccc(-c2nc(-c3ccc4ccccc4c3)nc(-c3ccc(-c4ccc(-c5ccc6ccccc6c5)cc4)c4oc5ccccc5c34)n2)cc1]

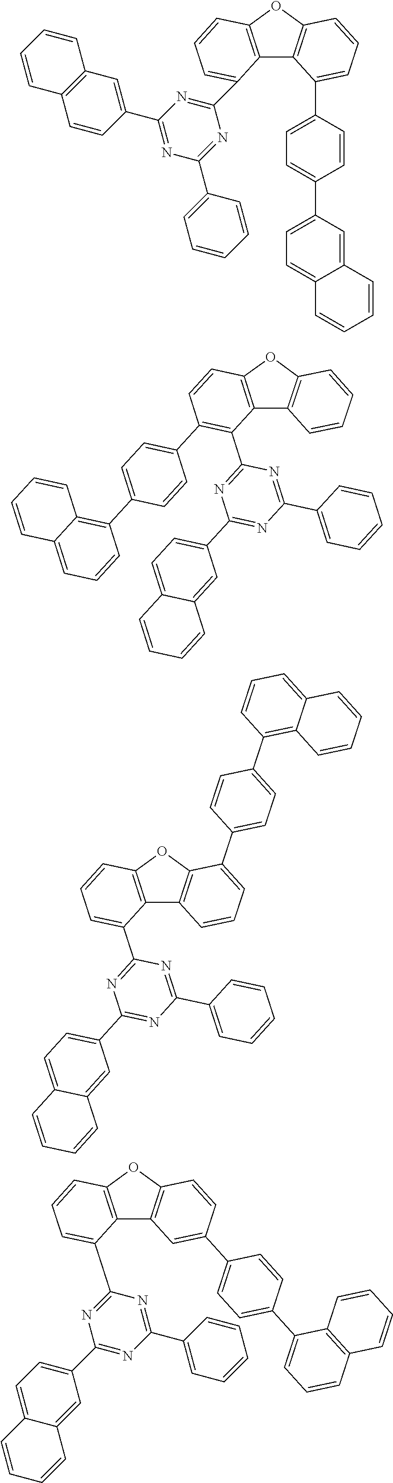

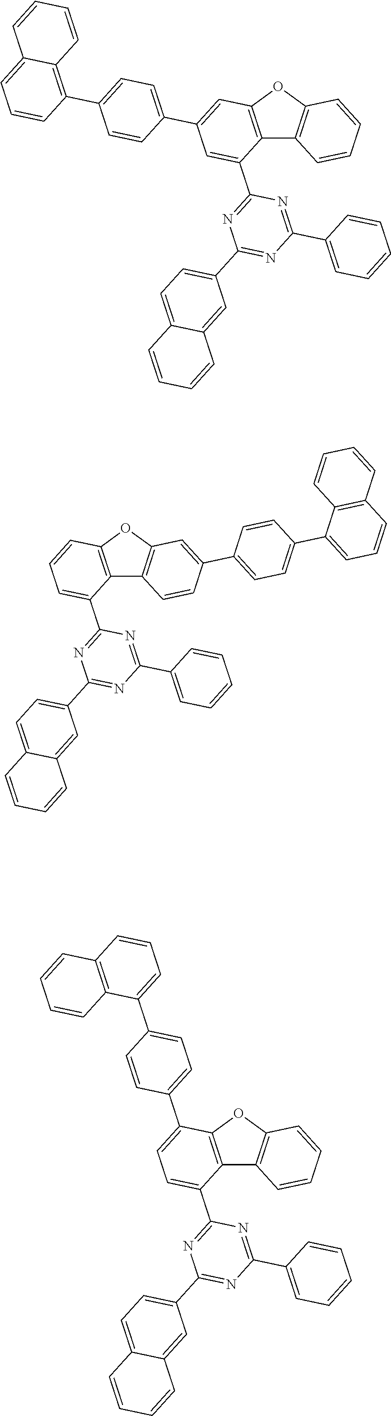 [SMILES: c1ccc(-c2nc(-c3ccc4ccccc4c3)nc(-c3cc(-c4ccc(-c5cccc6ccccc56)cc4)cc4oc5ccccc5c34)n2)cc1 ; c1ccc(-c2nc(-c3ccc4ccccc4c3)nc(-c3cccc4oc5cc(-c6ccc(-c7cccc8ccccc78)cc6)ccc5c34)n2)cc1 ; c1ccc(-c2nc(-c3ccc4ccccc4c3)nc(-c3ccc(-c4ccc(-c5cccc6ccccc56)cc4)c4oc5ccccc5c34)n2)cc1]

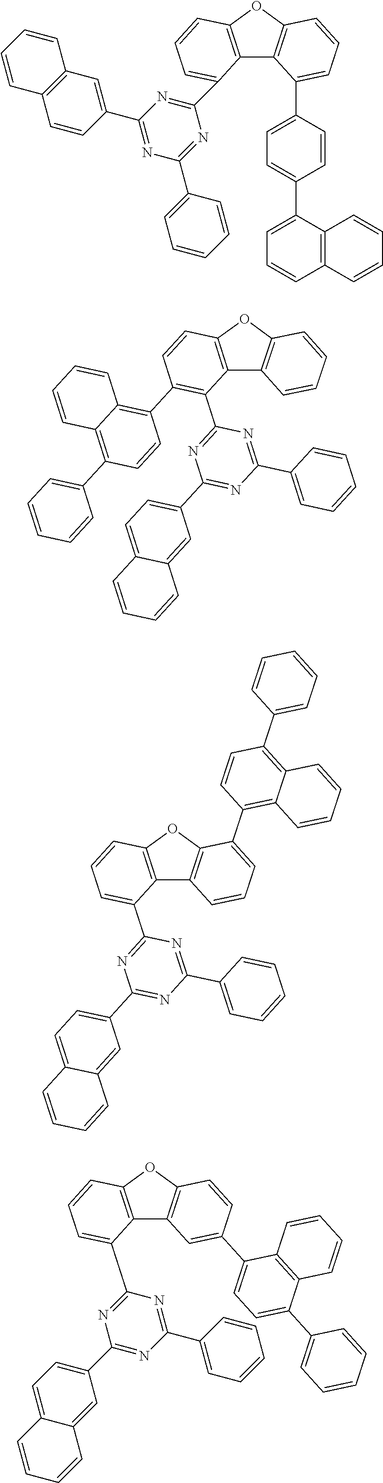 [SMILES: c1ccc(-c2nc(-c3ccc4ccccc4c3)nc(-c3cccc4oc5cccc(-c6ccc(-c7cccc8ccccc78)cc6)c5c34)n2)cc1 ; c1ccc(-c2nc(-c3ccc4ccccc4c3)nc(-c3c(-c4ccc(-c5ccccc5)c5ccccc45)ccc4oc5ccccc5c34)n2)cc1 ; c1ccc(-c2nc(-c3ccc4ccccc4c3)nc(-c3cccc4oc5c(-c6ccc(-c7ccccc7)c7ccccc67)cccc5c34)n2)cc1 ; c1ccc(-c2nc(-c3ccc4ccccc4c3)nc(-c3cccc4oc5ccc(-c6ccc(-c7ccccc7)c7ccccc67)cc5c34)n2)cc1]

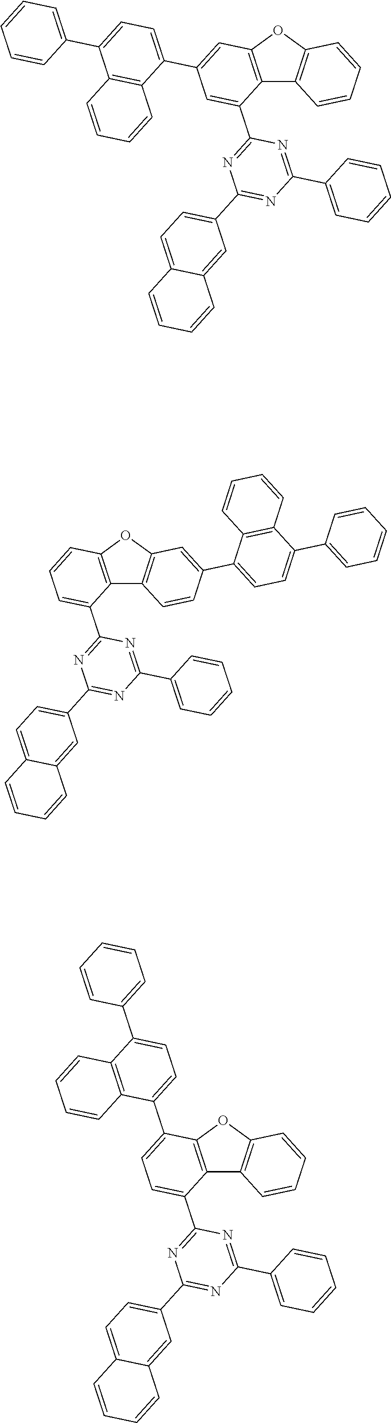 [SMILES: c1ccc(-c2nc(-c3ccc4ccccc4c3)nc(-c3cc(-c4ccc(-c5ccccc5)c5ccccc45)cc4oc5ccccc5c34)n2)cc1 ; c1ccc(-c2nc(-c3ccc4ccccc4c3)nc(-c3cccc4oc5cc(-c6ccc(-c7ccccc7)c7ccccc67)ccc5c34)n2)cc1 ; c1ccc(-c2nc(-c3ccc4ccccc4c3)nc(-c3ccc(-c4ccc(-c5ccccc5)c5ccccc45)c4oc5ccccc5c34)n2)cc1]

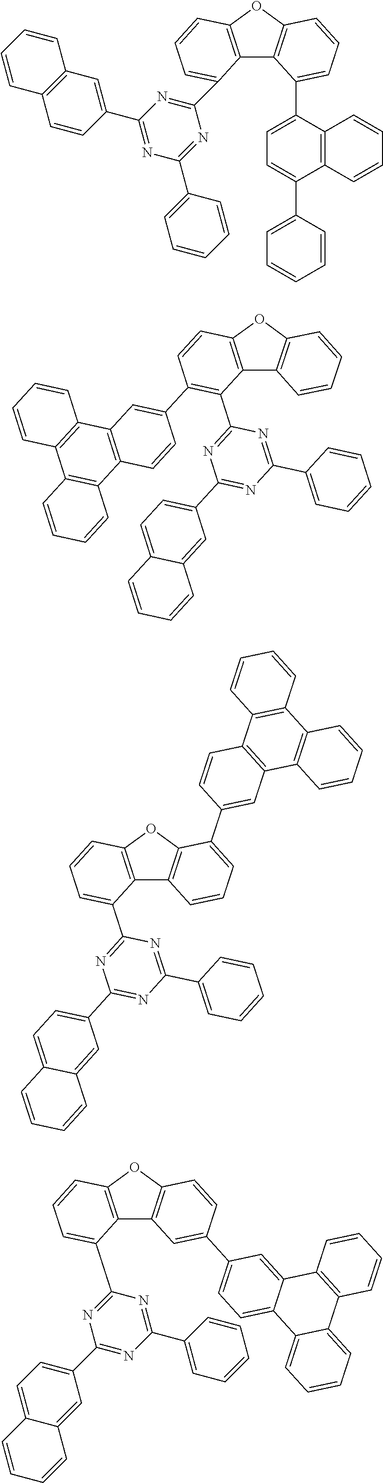 [SMILES: c1ccc(-c2nc(-c3ccc4ccccc4c3)nc(-c3cccc4oc5cccc(-c6ccc(-c7ccccc7)c7ccccc67)c5c34)n2)cc1 ; c1ccc(-c2nc(-c3ccc4ccccc4c3)nc(-c3c(-c4ccc5c6ccccc6c6ccccc6c5c4)ccc4oc5ccccc5c34)n2)cc1 ; c1ccc(-c2nc(-c3ccc4ccccc4c3)nc(-c3cccc4oc5c(-c6ccc7c8ccccc8c8ccccc8c7c6)cccc5c34)n2)cc1 ; c1ccc(-c2nc(-c3ccc4ccccc4c3)nc(-c3cccc4oc5ccc(-c6ccc7c8ccccc8c8ccccc8c7c6)cc5c34)n2)cc1]

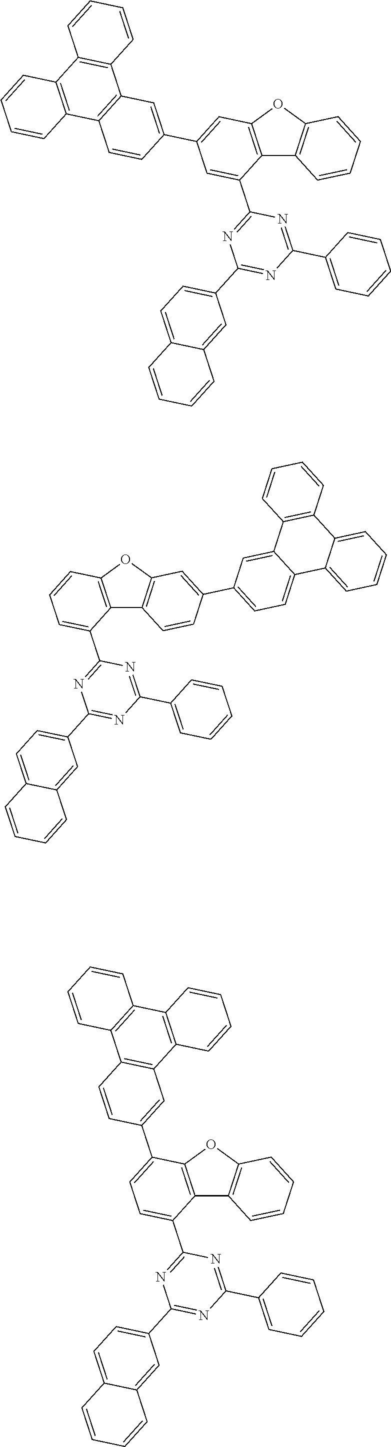 [SMILES: c1ccc(-c2nc(-c3ccc4ccccc4c3)nc(-c3cc(-c4ccc5c6ccccc6c6ccccc6c5c4)cc4oc5ccccc5c34)n2)cc1 ; c1ccc(-c2nc(-c3ccc4ccccc4c3)nc(-c3cccc4oc5cc(-c6ccc7c8ccccc8c8ccccc8c7c6)ccc5c34)n2)cc1 ; c1ccc(-c2nc(-c3ccc4ccccc4c3)nc(-c3ccc(-c4ccc5c6ccccc6c6ccccc6c5c4)c4oc5ccccc5c34)n2)cc1]

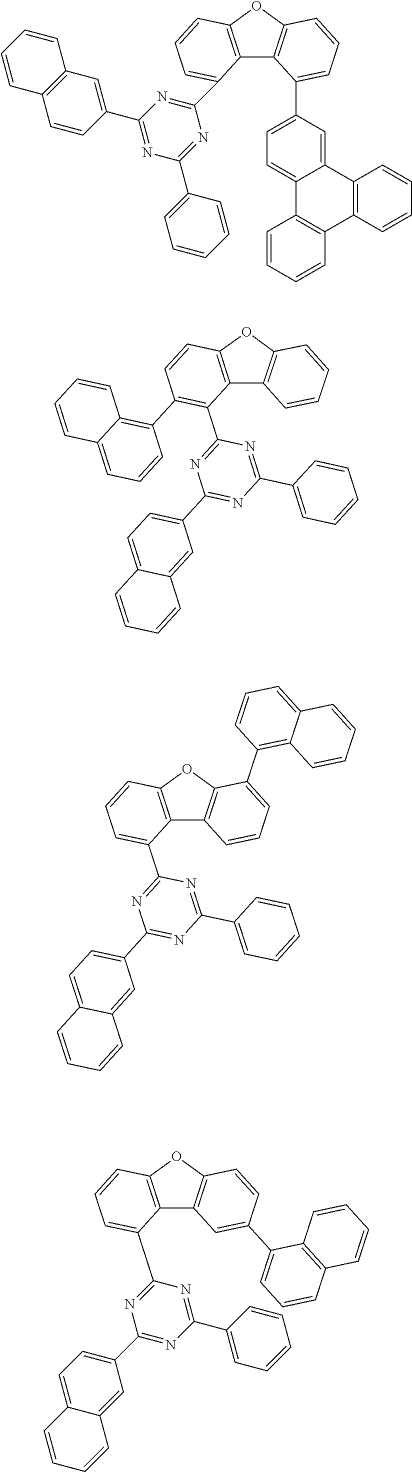 [SMILES: c1ccc(-c2nc(-c3ccc4ccccc4c3)nc(-c3cccc4oc5cccc(-c6ccc7c8ccccc8c8ccccc8c7c6)c5c34)n2)cc1 ; c1ccc(-c2nc(-c3ccc4ccccc4c3)nc(-c3c(-c4cccc5ccccc45)ccc4oc5ccccc5c34)n2)cc1 ; c1ccc(-c2nc(-c3ccc4ccccc4c3)nc(-c3cccc4oc5c(-c6cccc7ccccc67)cccc5c34)n2)cc1 ; c1ccc(-c2nc(-c3ccc4ccccc4c3)nc(-c3cccc4oc5ccc(-c6cccc7ccccc67)cc5c34)n2)cc1]

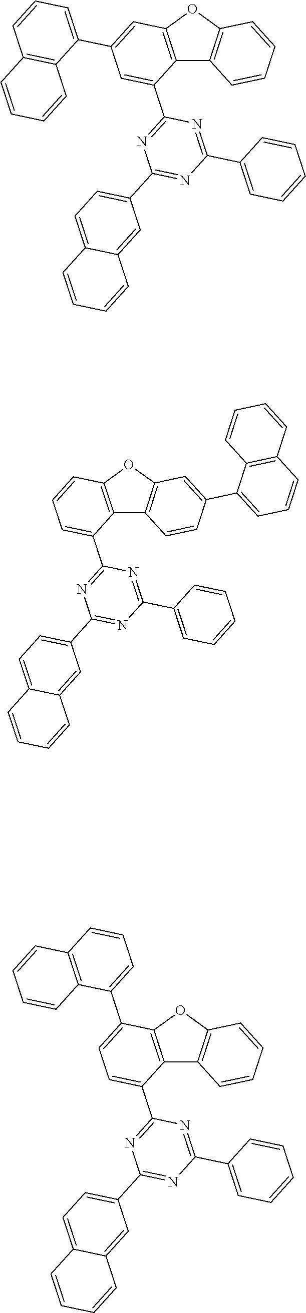 [SMILES: c1ccc(-c2nc(-c3ccc4ccccc4c3)nc(-c3cc(-c4cccc5ccccc45)cc4oc5ccccc5c34)n2)cc1 ; c1ccc(-c2nc(-c3ccc4ccccc4c3)nc(-c3cccc4oc5cc(-c6cccc7ccccc67)ccc5c34)n2)cc1 ; c1ccc(-c2nc(-c3ccc4ccccc4c3)nc(-c3ccc(-c4cccc5ccccc45)c4oc5ccccc5c34)n2)cc1]

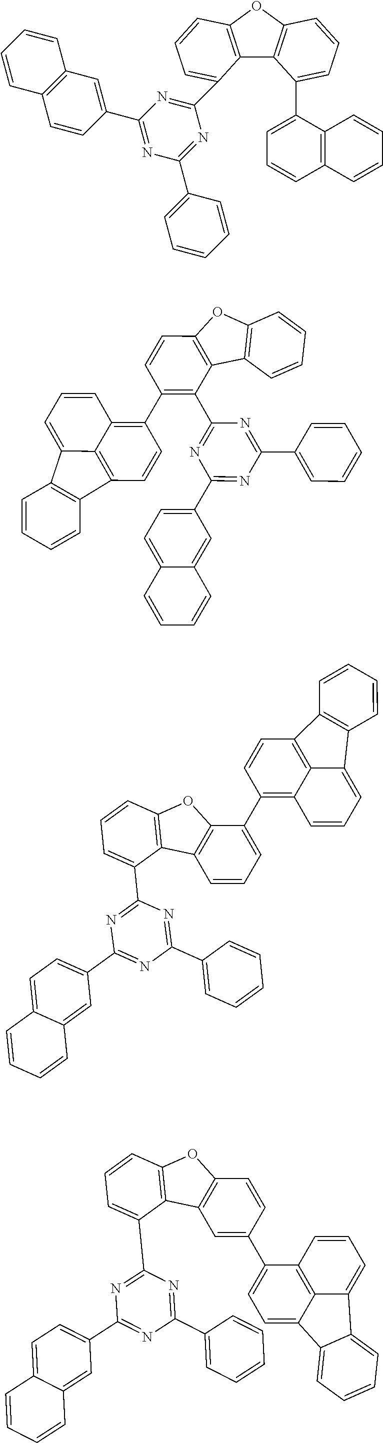 [SMILES: c1ccc(-c2nc(-c3ccc4ccccc4c3)nc(-c3cccc4oc5cccc(-c6cccc7ccccc67)c5c34)n2)cc1 ; c1ccc(-c2nc(-c3ccc4ccccc4c3)nc(-c3c(-c4ccc5c6c(cccc46)-c4ccccc4-5)ccc4oc5ccccc5c34)n2)cc1 ; c1ccc(-c2nc(-c3ccc4ccccc4c3)nc(-c3cccc4oc5c(-c6ccc7c8c(cccc68)-c6ccccc6-7)cccc5c34)n2)cc1 ; c1ccc(-c2nc(-c3ccc4ccccc4c3)nc(-c3cccc4oc5ccc(-c6ccc7c8c(cccc68)-c6ccccc6-7)cc5c34)n2)cc1]

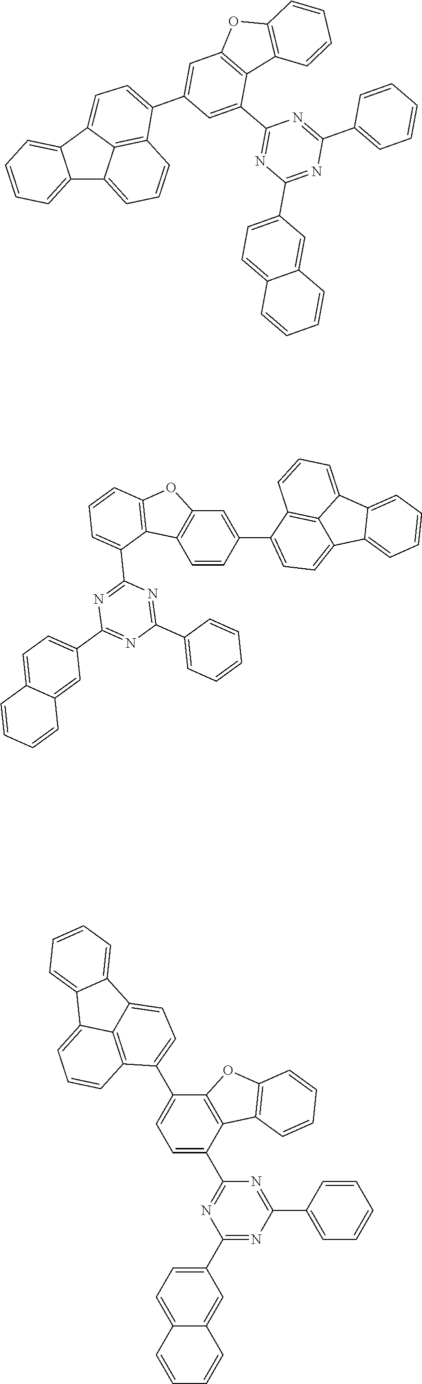 [SMILES: c1ccc(-c2nc(-c3ccc4ccccc4c3)nc(-c3cc(-c4ccc5c6c(cccc46)-c4ccccc4-5)cc4oc5ccccc5c34)n2)cc1 ; c1ccc(-c2nc(-c3ccc4ccccc4c3)nc(-c3cccc4oc5cc(-c6ccc7c8c(cccc68)-c6ccccc6-7)ccc5c34)n2)cc1 ; c1ccc(-c2nc(-c3ccc4ccccc4c3)nc(-c3ccc(-c4ccc5c6c(cccc46)-c4ccccc4-5)c4oc5ccccc5c34)n2)cc1]

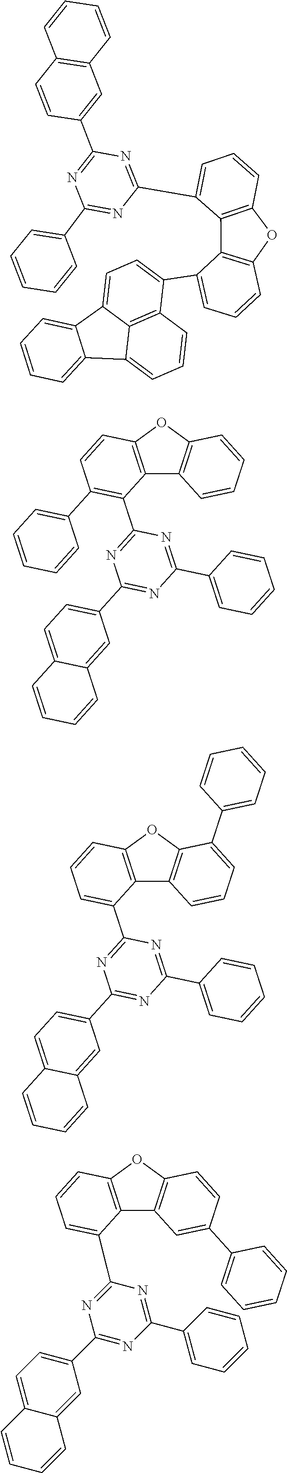

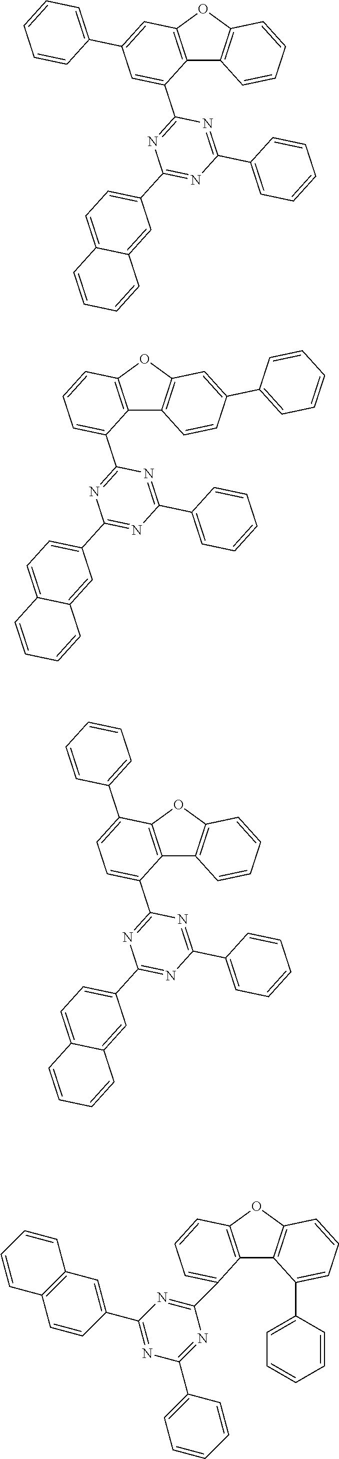 [SMILES: c1ccc(-c2cc(-c3nc(-c4ccccc4)nc(-c4ccc5ccccc5c4)n3)c3c(c2)oc2ccccc23)cc1 ; c1ccc(-c2ccc3c(c2)oc2cccc(-c4nc(-c5ccccc5)nc(-c5ccc6ccccc6c5)n4)c23)cc1 ; c1ccc(-c2nc(-c3ccc4ccccc4c3)nc(-c3ccc(-c4ccccc4)c4oc5ccccc5c34)n2)cc1 ; c1ccc(-c2nc(-c3ccc4ccccc4c3)nc(-c3cccc4oc5cccc(-c6ccccc6)c5c34)n2)cc1]

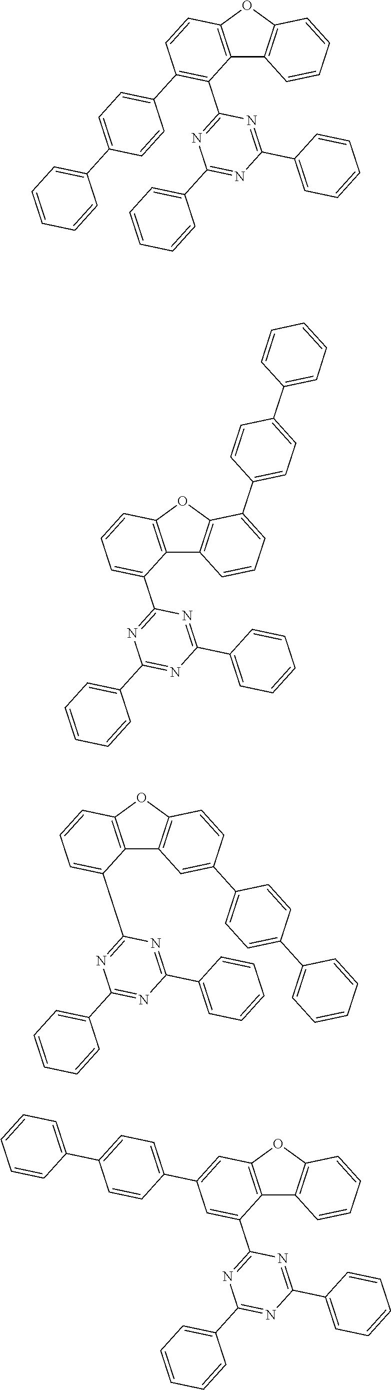 [SMILES: c1ccc(-c2ccc(-c3ccc4oc5ccccc5c4c3-c3nc(-c4ccccc4)nc(-c4ccccc4)n3)cc2)cc1 ; c1ccc(-c2ccc(-c3cccc4c3oc3cccc(-c5nc(-c6ccccc6)nc(-c6ccccc6)n5)c34)cc2)cc1 ; c1ccc(-c2ccc(-c3ccc4oc5cccc(-c6nc(-c7ccccc7)nc(-c7ccccc7)n6)c5c4c3)cc2)cc1 ; c1ccc(-c2ccc(-c3cc(-c4nc(-c5ccccc5)nc(-c5ccccc5)n4)c4c(c3)oc3ccccc34)cc2)cc1]

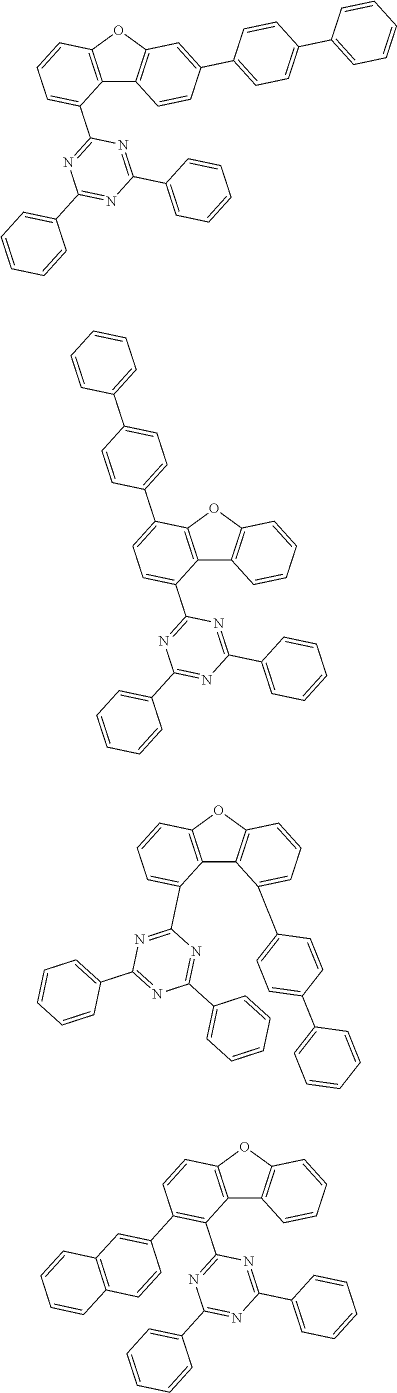 [SMILES: c1ccc(-c2ccc(-c3ccc4c(c3)oc3cccc(-c5nc(-c6ccccc6)nc(-c6ccccc6)n5)c34)cc2)cc1 ; c1ccc(-c2ccc(-c3ccc(-c4nc(-c5ccccc5)nc(-c5ccccc5)n4)c4c3oc3ccccc34)cc2)cc1 ; c1ccc(-c2ccc(-c3cccc4oc5cccc(-c6nc(-c7ccccc7)nc(-c7ccccc7)n6)c5c34)cc2)cc1 ; c1ccc(-c2nc(-c3ccccc3)nc(-c3c(-c4ccc5ccccc5c4)ccc4oc5ccccc5c34)n2)cc1]

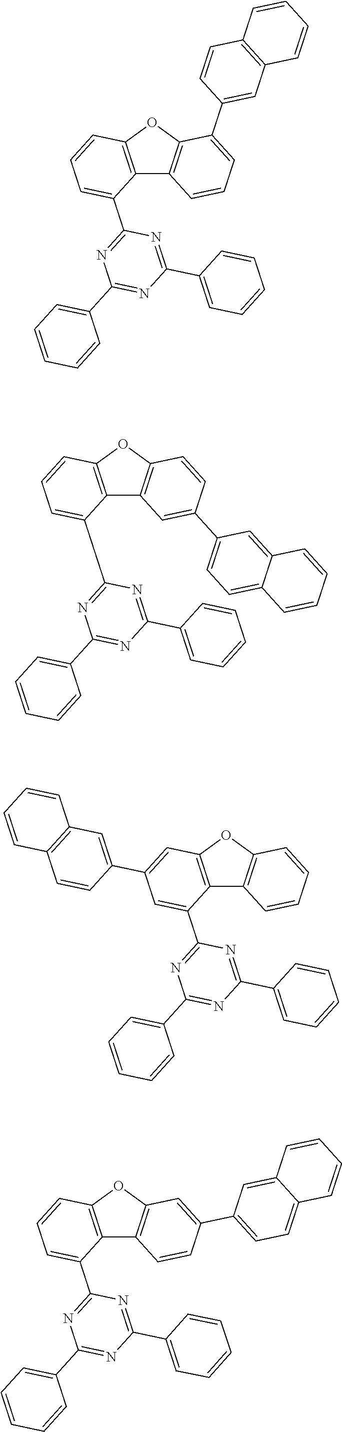 [SMILES: c1ccc(-c2nc(-c3ccccc3)nc(-c3cccc4oc5c(-c6ccc7ccccc7c6)cccc5c34)n2)cc1 ; c1ccc(-c2nc(-c3ccccc3)nc(-c3cccc4oc5ccc(-c6ccc7ccccc7c6)cc5c34)n2)cc1 ; c1ccc(-c2nc(-c3ccccc3)nc(-c3cc(-c4ccc5ccccc5c4)cc4oc5ccccc5c34)n2)cc1 ; c1ccc(-c2nc(-c3ccccc3)nc(-c3cccc4oc5cc(-c6ccc7ccccc7c6)ccc5c34)n2)cc1]

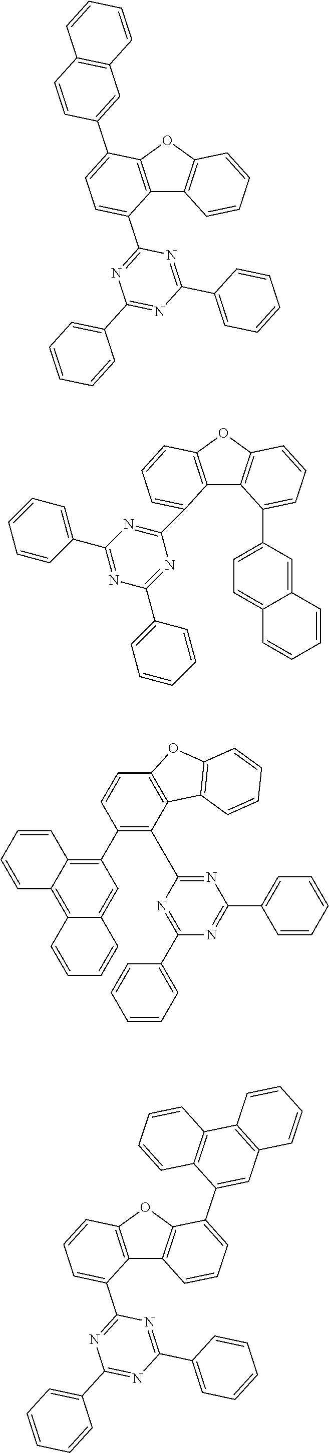 [SMILES: c1ccc(-c2nc(-c3ccccc3)nc(-c3ccc(-c4ccc5ccccc5c4)c4oc5ccccc5c34)n2)cc1 ; c1ccc(-c2nc(-c3ccccc3)nc(-c3cccc4oc5cccc(-c6ccc7ccccc7c6)c5c34)n2)cc1 ; c1ccc(-c2nc(-c3ccccc3)nc(-c3c(-c4cc5ccccc5c5ccccc45)ccc4oc5ccccc5c34)n2)cc1 ; c1ccc(-c2nc(-c3ccccc3)nc(-c3cccc4oc5c(-c6cc7ccccc7c7ccccc67)cccc5c34)n2)cc1]

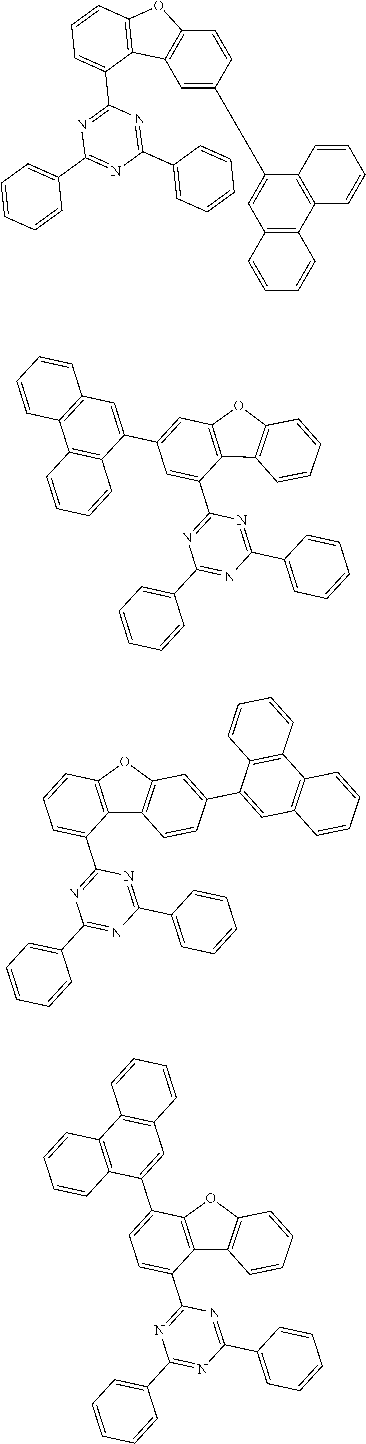 [SMILES: c1ccc(-c2nc(-c3ccccc3)nc(-c3cccc4oc5ccc(-c6cc7ccccc7c7ccccc67)cc5c34)n2)cc1 ; c1ccc(-c2nc(-c3ccccc3)nc(-c3cc(-c4cc5ccccc5c5ccccc45)cc4oc5ccccc5c34)n2)cc1 ; c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3ccc(-c4cc5ccccc5c5ccccc45)c4oc5ccccc5c34)n2)cc1]

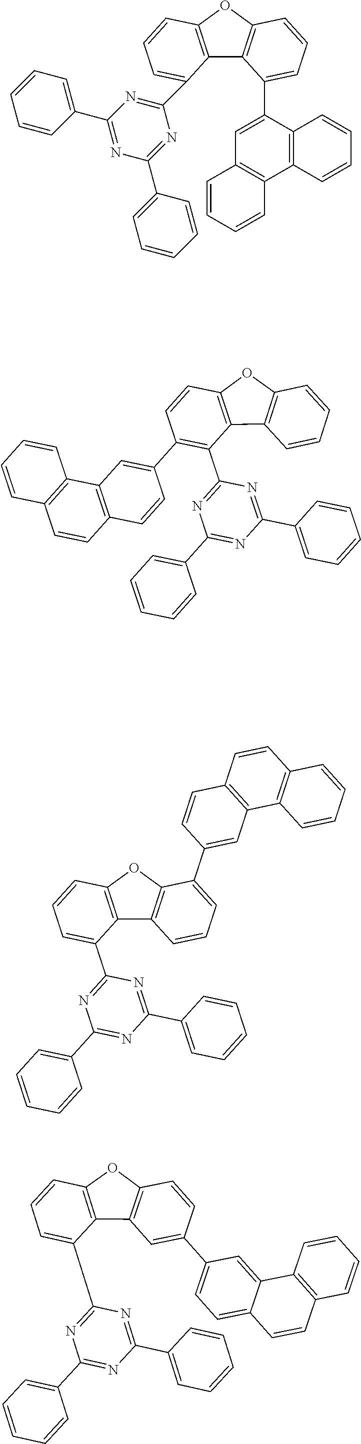 [SMILES: c1ccc(-c2nc(-c3ccccc3)nc(-c3cccc4oc5cccc(-c6cc7ccccc7c7ccccc67)c5c34)n2)cc1 ; c1ccc(-c2nc(-c3ccccc3)nc(-c3c(-c4ccc5ccc6ccccc6c5c4)ccc4oc5ccccc5c34)n2)cc1 ; c1ccc(-c2nc(-c3ccccc3)nc(-c3cccc4oc5c(-c6ccc7ccc8ccccc8c7c6)cccc5c34)n2)cc1 ; c1ccc(-c2nc(-c3ccccc3)nc(-c3cccc4oc5ccc(-c6ccc7ccc8ccccc8c7c6)cc5c34)n2)cc1]

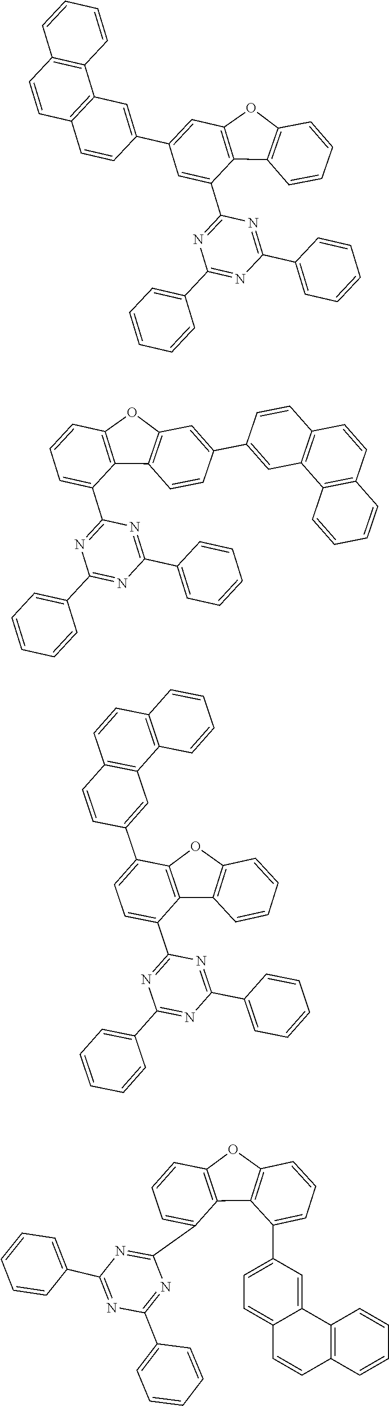 [SMILES: c1ccc(-c2nc(-c3ccccc3)nc(-c3cc(-c4ccc5ccc6ccccc6c5c4)cc4oc5ccccc5c34)n2)cc1 ; c1ccc(-c2nc(-c3ccccc3)nc(-c3cccc4oc5cc(-c6ccc7ccc8ccccc8c7c6)ccc5c34)n2)cc1 ; c1ccc(-c2nc(-c3ccccc3)nc(-c3ccc(-c4ccc5ccc6ccccc6c5c4)c4oc5ccccc5c34)n2)cc1 ; c1ccc(-c2nc(-c3ccccc3)nc(-c3cccc4oc5cccc(-c6ccc7ccc8ccccc8c7c6)c5c34)n2)cc1]

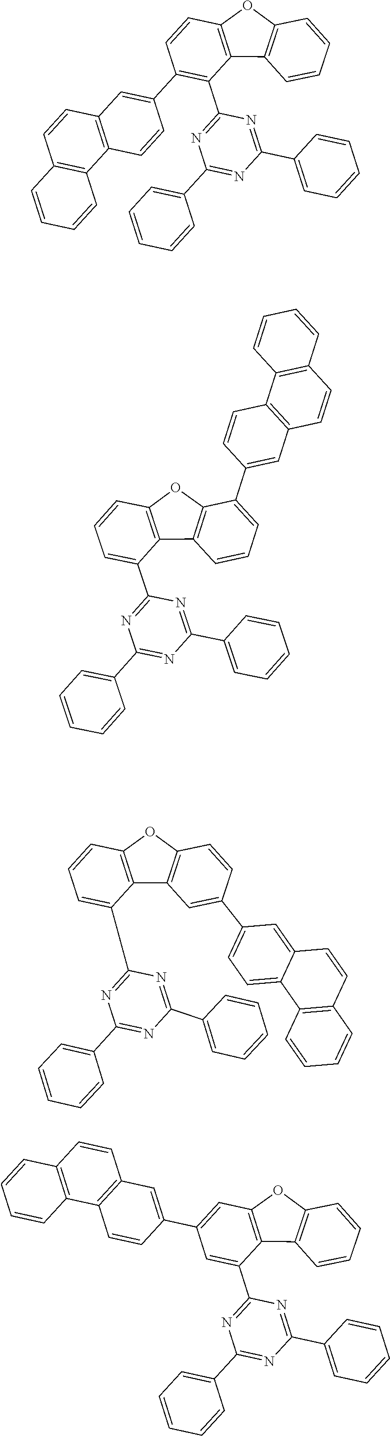

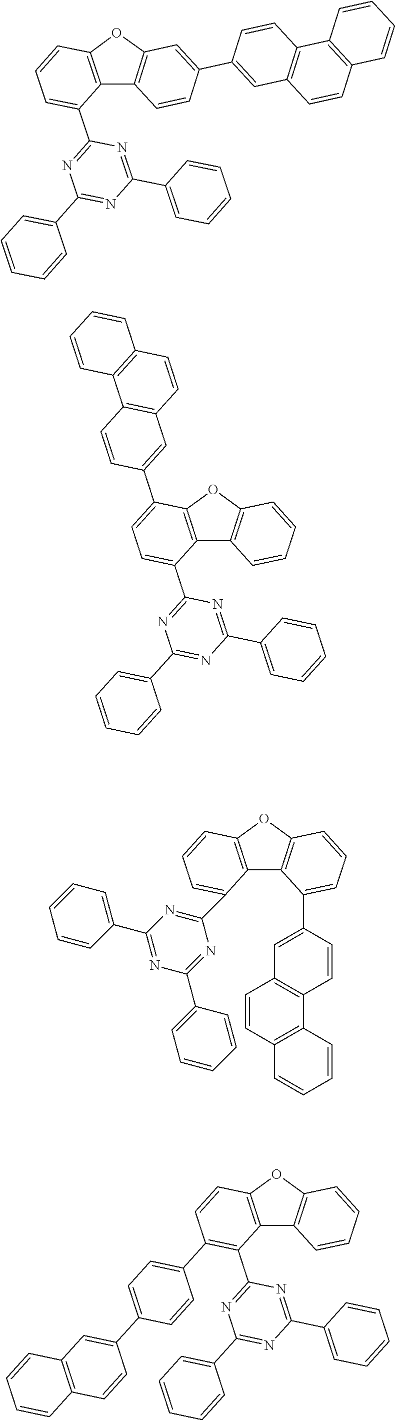 [SMILES: c1ccc(-c2nc(-c3ccccc3)nc(-c3cccc4oc5cc(-c6ccc7c(ccc8ccccc87)c6)ccc5c34)n2)cc1 ; c1ccc(-c2nc(-c3ccccc3)nc(-c3ccc(-c4ccc5c(ccc6ccccc65)c4)c4oc5ccccc5c34)n2)cc1 ; c1ccc(-c2nc(-c3ccccc3)nc(-c3cccc4oc5cccc(-c6ccc7c(ccc8ccccc87)c6)c5c34)n2)cc1 ; c1ccc(-c2nc(-c3ccccc3)nc(-c3c(-c4ccc(-c5ccc6ccccc6c5)cc4)ccc4oc5ccccc5c34)n2)cc1]

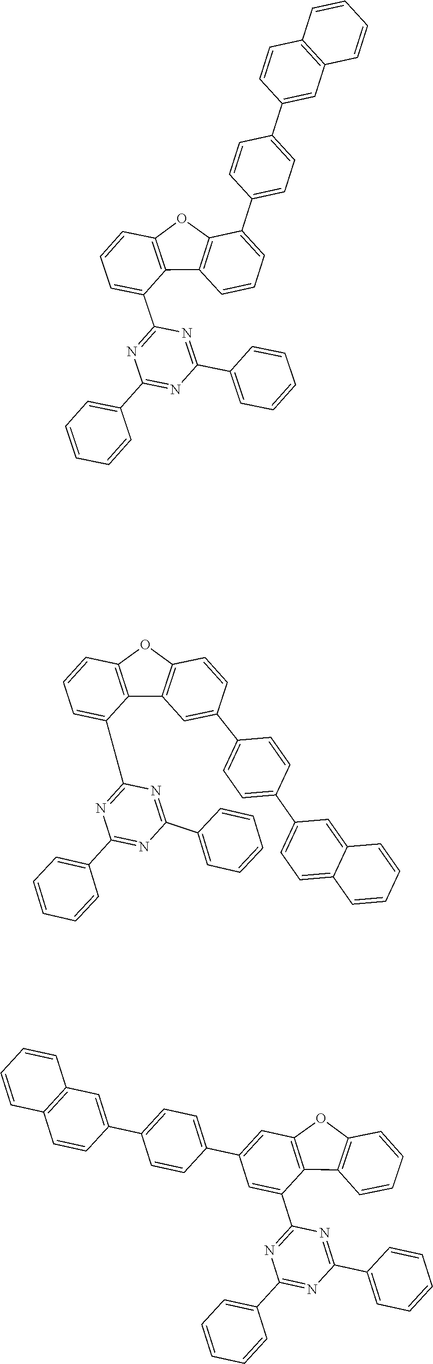 [SMILES: c1ccc(-c2nc(-c3ccccc3)nc(-c3cccc4oc5c(-c6ccc(-c7ccc8ccccc8c7)cc6)cccc5c34)n2)cc1 ; c1ccc(-c2nc(-c3ccccc3)nc(-c3cccc4oc5ccc(-c6ccc(-c7ccc8ccccc8c7)cc6)cc5c34)n2)cc1 ; c1ccc(-c2nc(-c3ccccc3)nc(-c3cc(-c4ccc(-c5ccc6ccccc6c5)cc4)cc4oc5ccccc5c34)n2)cc1]

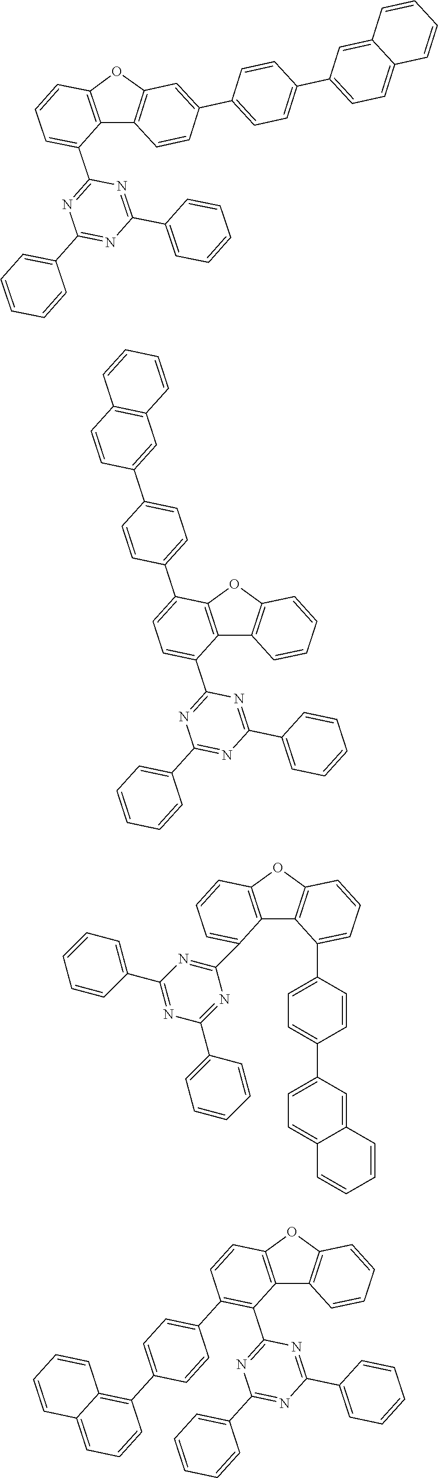 [SMILES: c1ccc(-c2nc(-c3ccccc3)nc(-c3cccc4oc5cc(-c6ccc(-c7ccc8ccccc8c7)cc6)ccc5c34)n2)cc1 ; c1ccc(-c2nc(-c3ccccc3)nc(-c3ccc(-c4ccc(-c5ccc6ccccc6c5)cc4)c4oc5ccccc5c34)n2)cc1 ; c1ccc(-c2nc(-c3ccccc3)nc(-c3cccc4oc5cccc(-c6ccc(-c7ccc8ccccc8c7)cc6)c5c34)n2)cc1 ; c1ccc(-c2nc(-c3ccccc3)nc(-c3c(-c4ccc(-c5cccc6ccccc56)cc4)ccc4oc5ccccc5c34)n2)cc1]

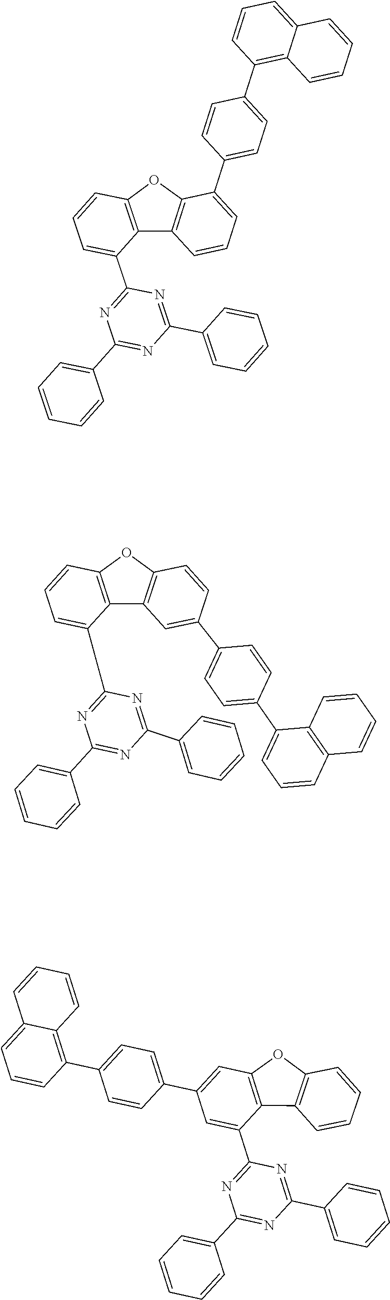 [SMILES: c1ccc(-c2nc(-c3ccccc3)nc(-c3cccc4oc5c(-c6ccc(-c7cccc8ccccc78)cc6)cccc5c34)n2)cc1 ; c1ccc(-c2nc(-c3ccccc3)nc(-c3cccc4oc5ccc(-c6ccc(-c7cccc8ccccc78)cc6)cc5c34)n2)cc1 ; c1ccc(-c2nc(-c3ccccc3)nc(-c3cc(-c4ccc(-c5cccc6ccccc56)cc4)cc4oc5ccccc5c34)n2)cc1]

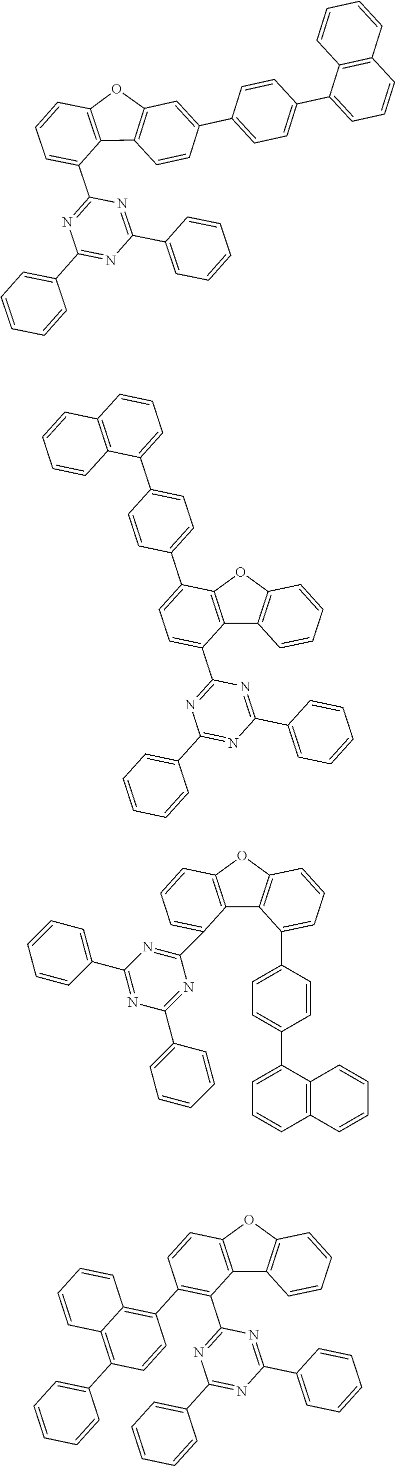 [SMILES: c1ccc(-c2nc(-c3ccccc3)nc(-c3cccc4oc5cc(-c6ccc(-c7cccc8ccccc78)cc6)ccc5c34)n2)cc1 ; c1ccc(-c2nc(-c3ccccc3)nc(-c3ccc(-c4ccc(-c5cccc6ccccc56)cc4)c4oc5ccccc5c34)n2)cc1 ; c1ccc(-c2nc(-c3ccccc3)nc(-c3cccc4oc5cccc(-c6ccc(-c7cccc8ccccc78)cc6)c5c34)n2)cc1 ; c1ccc(-c2nc(-c3ccccc3)nc(-c3c(-c4ccc(-c5ccccc5)c5ccccc45)ccc4oc5ccccc5c34)n2)cc1]

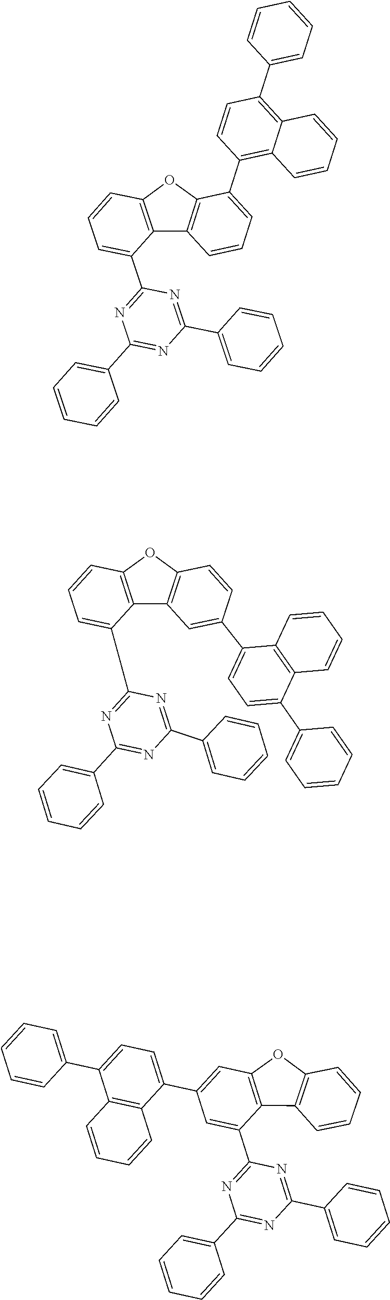 [SMILES: c1ccc(-c2nc(-c3ccccc3)nc(-c3cccc4oc5c(-c6ccc(-c7ccccc7)c7ccccc67)cccc5c34)n2)cc1 ; c1ccc(-c2nc(-c3ccccc3)nc(-c3cccc4oc5ccc(-c6ccc(-c7ccccc7)c7ccccc67)cc5c34)n2)cc1 ; c1ccc(-c2nc(-c3ccccc3)nc(-c3cc(-c4ccc(-c5ccccc5)c5ccccc45)cc4oc5ccccc5c34)n2)cc1]

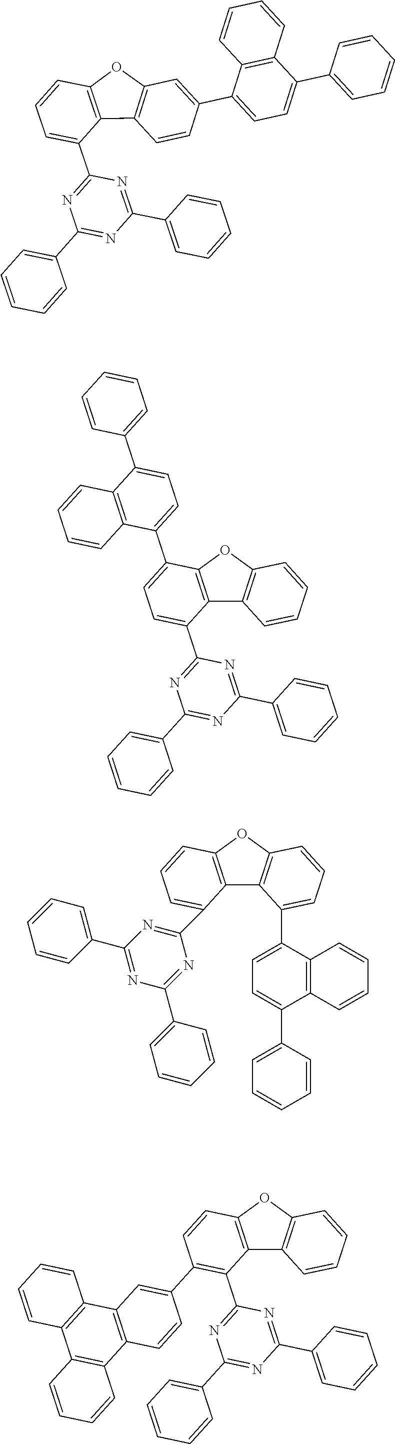 [SMILES: c1ccc(-c2nc(-c3ccccc3)nc(-c3cccc4oc5cc(-c6ccc(-c7ccccc7)c7ccccc67)ccc5c34)n2)cc1 ; c1ccc(-c2nc(-c3ccccc3)nc(-c3ccc(-c4ccc(-c5ccccc5)c5ccccc45)c4oc5ccccc5c34)n2)cc1 ; c1ccc(-c2nc(-c3ccccc3)nc(-c3cccc4oc5cccc(-c6ccc(-c7ccccc7)c7ccccc67)c5c34)n2)cc1 ; c1ccc(-c2nc(-c3ccccc3)nc(-c3c(-c4ccc5c6ccccc6c6ccccc6c5c4)ccc4oc5ccccc5c34)n2)cc1]

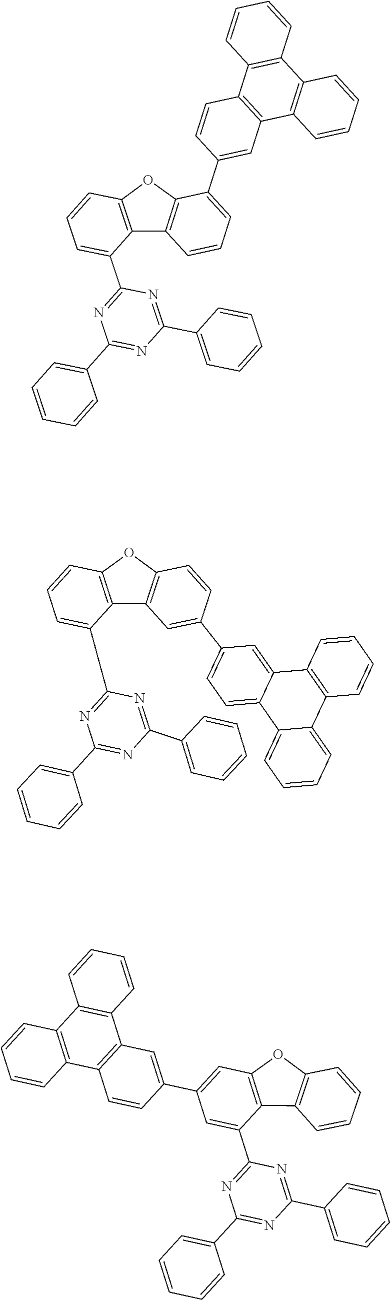 [SMILES: c1ccc(-c2nc(-c3ccccc3)nc(-c3cccc4oc5c(-c6ccc7c8ccccc8c8ccccc8c7c6)cccc5c34)n2)cc1 ; c1ccc(-c2nc(-c3ccccc3)nc(-c3cccc4oc5ccc(-c6ccc7c8ccccc8c8ccccc8c7c6)cc5c34)n2)cc1 ; c1ccc(-c2nc(-c3ccccc3)nc(-c3cc(-c4ccc5c6ccccc6c6ccccc6c5c4)cc4oc5ccccc5c34)n2)cc1]

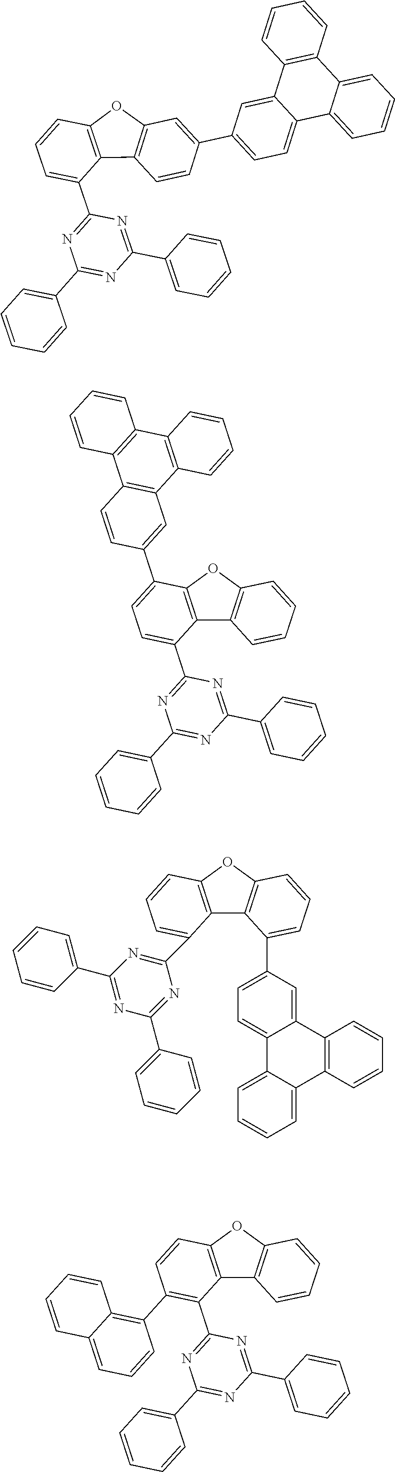 [SMILES: c1ccc(-c2nc(-c3ccccc3)nc(-c3cccc4oc5cc(-c6ccc7c8ccccc8c8ccccc8c7c6)ccc5c34)n2)cc1 ; c1ccc(-c2nc(-c3ccccc3)nc(-c3ccc(-c4ccc5c6ccccc6c6ccccc6c5c4)c4oc5ccccc5c34)n2)cc1 ; c1ccc(-c2nc(-c3ccccc3)nc(-c3cccc4oc5cccc(-c6ccc7c8ccccc8c8ccccc8c7c6)c5c34)n2)cc1 ; c1ccc(-c2nc(-c3ccccc3)nc(-c3c(-c4cccc5ccccc45)ccc4oc5ccccc5c34)n2)cc1]

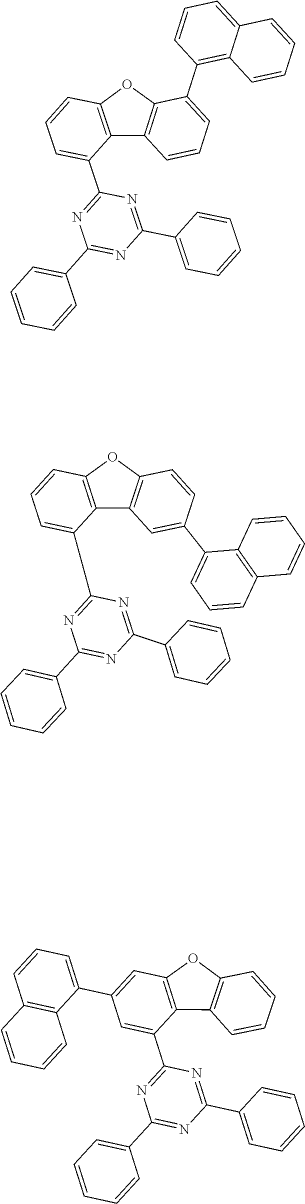 [SMILES: c1ccc(-c2nc(-c3ccccc3)nc(-c3cccc4oc5c(-c6cccc7ccccc67)cccc5c34)n2)cc1 ; c1ccc(-c2nc(-c3ccccc3)nc(-c3cccc4oc5ccc(-c6cccc7ccccc67)cc5c34)n2)cc1 ; c1ccc(-c2nc(-c3ccccc3)nc(-c3cc(-c4cccc5ccccc45)cc4oc5ccccc5c34)n2)cc1]

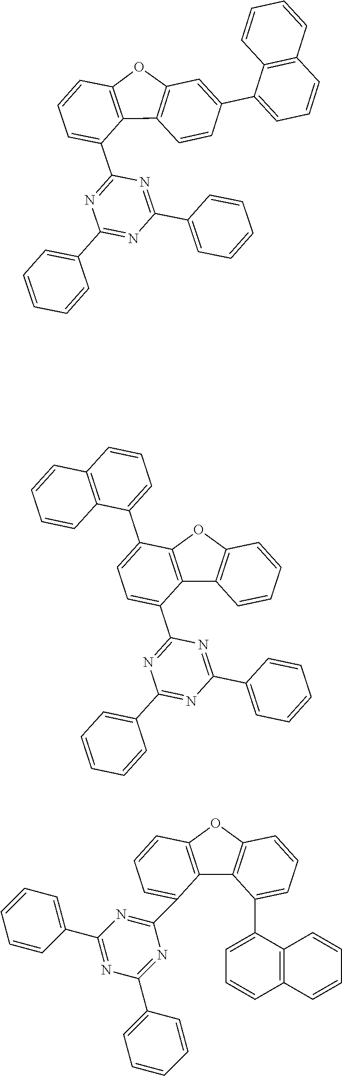 [SMILES: c1ccc(-c2nc(-c3ccccc3)nc(-c3cccc4oc5cc(-c6cccc7ccccc67)ccc5c34)n2)cc1 ; c1ccc(-c2nc(-c3ccccc3)nc(-c3ccc(-c4cccc5ccccc45)c4oc5ccccc5c34)n2)cc1 ; c1ccc(-c2nc(-c3ccccc3)nc(-c3cccc4oc5cccc(-c6cccc7ccccc67)c5c34)n2)cc1]

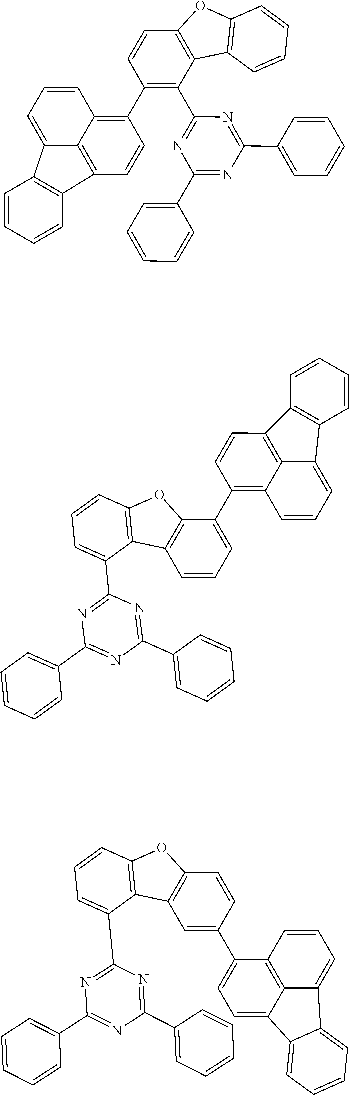 [SMILES: c1ccc(-c2nc(-c3ccccc3)nc(-c3c(-c4ccc5c6c(cccc46)-c4ccccc4-5)ccc4oc5ccccc5c34)n2)cc1 ; c1ccc(-c2nc(-c3ccccc3)nc(-c3cccc4oc5c(-c6ccc7c8c(cccc68)-c6ccccc6-7)cccc5c34)n2)cc1 ; c1ccc(-c2nc(-c3ccccc3)nc(-c3cccc4oc5ccc(-c6ccc7c8c(cccc68)-c6ccccc6-7)cc5c34)n2)cc1]

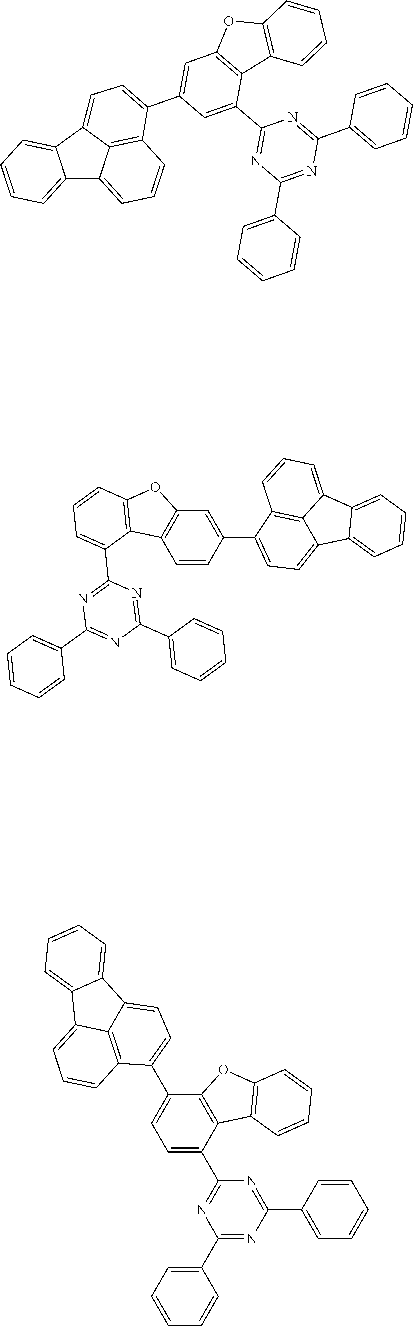 [SMILES: c1ccc(-c2nc(-c3ccccc3)nc(-c3cc(-c4ccc5c6c(cccc46)-c4ccccc4-5)cc4oc5ccccc5c34)n2)cc1 ; c1ccc(-c2nc(-c3ccccc3)nc(-c3cccc4oc5cc(-c6ccc7c8c(cccc68)-c6ccccc6-7)ccc5c34)n2)cc1 ; c1ccc(-c2nc(-c3ccccc3)nc(-c3ccc(-c4ccc5c6c(cccc46)-c4ccccc4-5)c4oc5ccccc5c34)n2)cc1]

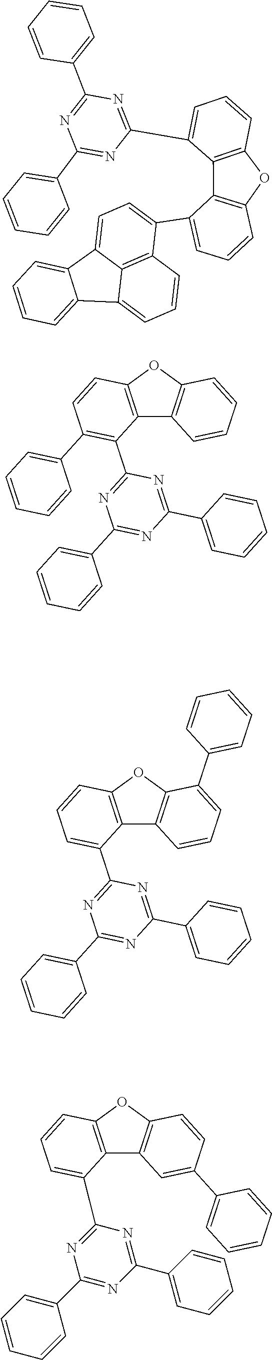

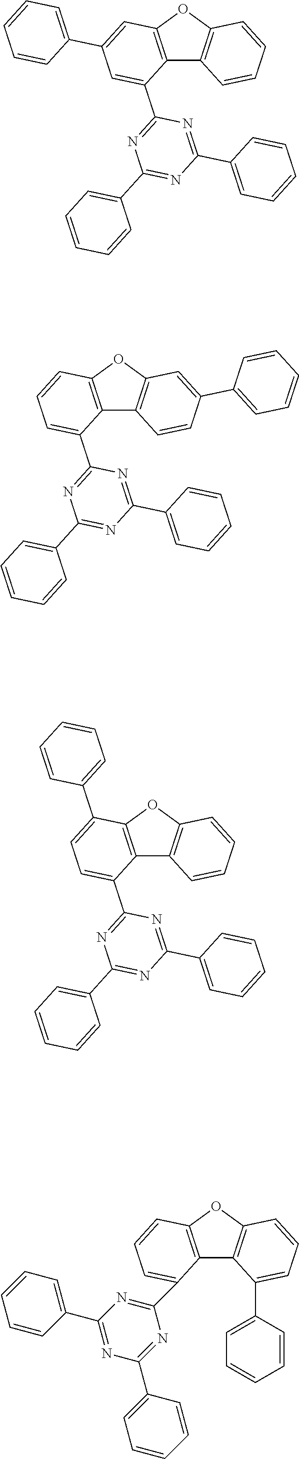 [SMILES: c1ccc(-c2cc(-c3nc(-c4ccccc4)nc(-c4ccccc4)n3)c3c(c2)oc2ccccc23)cc1 ; c1ccc(-c2ccc3c(c2)oc2cccc(-c4nc(-c5ccccc5)nc(-c5ccccc5)n4)c23)cc1 ; c1ccc(-c2nc(-c3ccccc3)nc(-c3ccc(-c4ccccc4)c4oc5ccccc5c34)n2)cc1 ; c1ccc(-c2nc(-c3ccccc3)nc(-c3cccc4oc5cccc(-c6ccccc6)c5c34)n2)cc1]

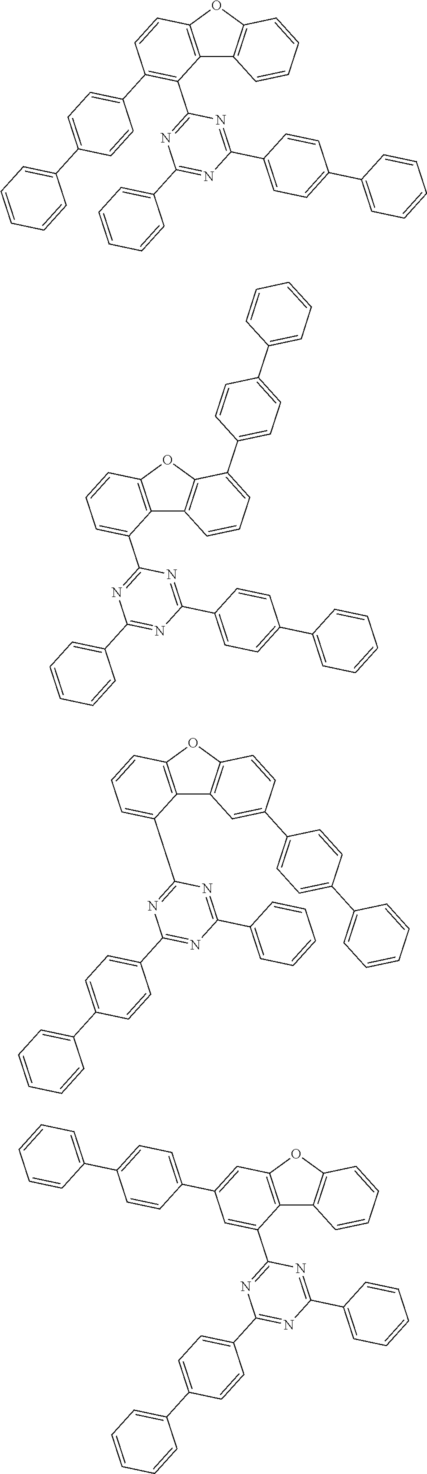

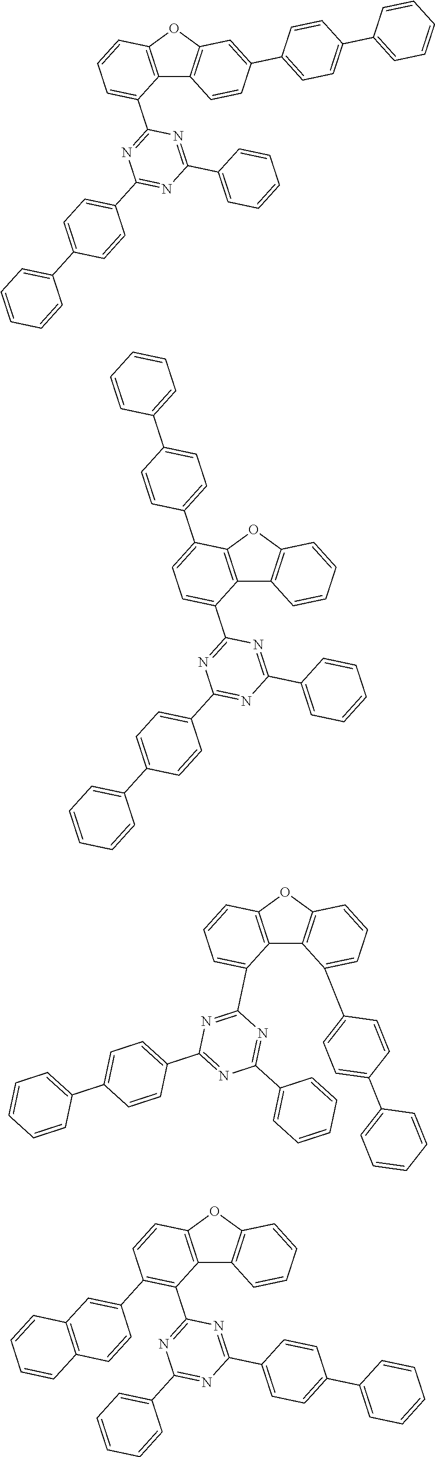 [SMILES: c1ccc(-c2ccc(-c3ccc4c(c3)oc3cccc(-c5nc(-c6ccccc6)nc(-c6ccc(-c7ccccc7)cc6)n5)c34)cc2)cc1 ; c1ccc(-c2ccc(-c3nc(-c4ccccc4)nc(-c4ccc(-c5ccc(-c6ccccc6)cc5)c5oc6ccccc6c45)n3)cc2)cc1 ; c1ccc(-c2ccc(-c3nc(-c4ccccc4)nc(-c4cccc5oc6cccc(-c7ccc(-c8ccccc8)cc7)c6c45)n3)cc2)cc1 ; c1ccc(-c2ccc(-c3nc(-c4ccccc4)nc(-c4c(-c5ccc6ccccc6c5)ccc5oc6ccccc6c45)n3)cc2)cc1]

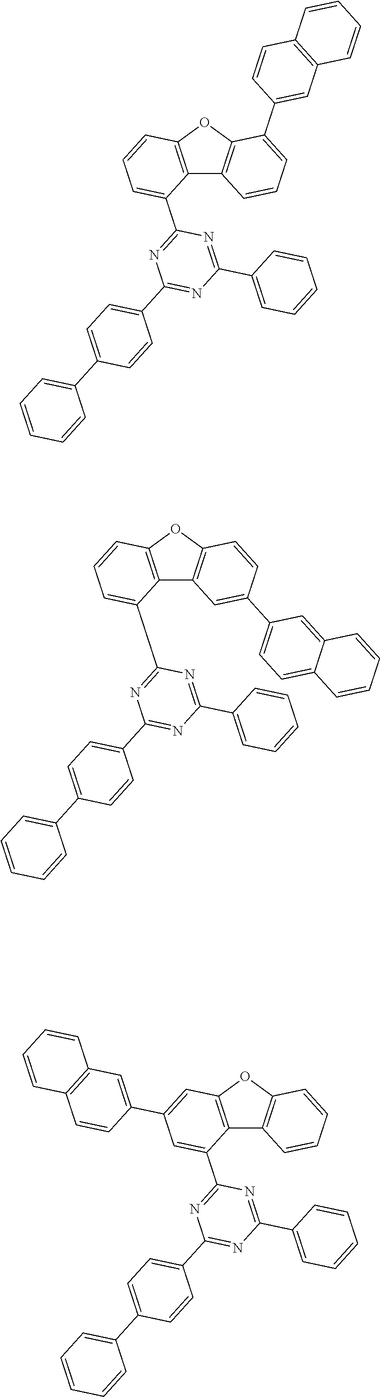 [SMILES: c1ccc(-c2ccc(-c3nc(-c4ccccc4)nc(-c4cccc5oc6c(-c7ccc8ccccc8c7)cccc6c45)n3)cc2)cc1 ; c1ccc(-c2ccc(-c3nc(-c4ccccc4)nc(-c4cccc5oc6ccc(-c7ccc8ccccc8c7)cc6c45)n3)cc2)cc1 ; c1ccc(-c2ccc(-c3nc(-c4ccccc4)nc(-c4cc(-c5ccc6ccccc6c5)cc5oc6ccccc6c45)n3)cc2)cc1]

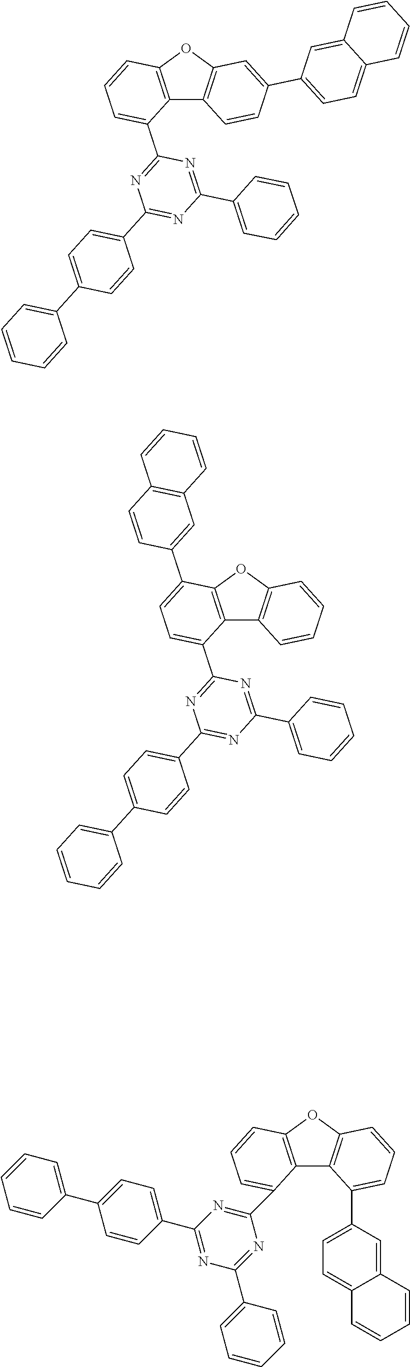 [SMILES: c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccccc4)nc(-c4ccc(-c5ccc6ccccc6c5)c5oc6ccccc6c45)n3)cc2)cc1 ; c1ccc(-c2ccc(-c3nc(-c4ccccc4)nc(-c4cccc5oc6cccc(-c7ccc8ccccc8c7)c6c45)n3)cc2)cc1]

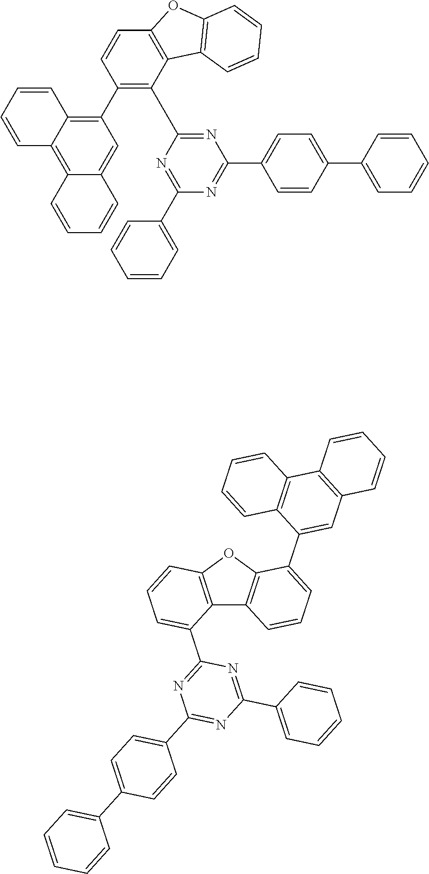 [SMILES: c1ccc(-c2ccc(-c3nc(-c4ccccc4)nc(-c4c(-c5cc6ccccc6c6ccccc56)ccc5oc6ccccc6c45)n3)cc2)cc1 ; c1ccc(-c2ccc(-c3nc(-c4ccccc4)nc(-c4cccc5oc6c(-c7cc8ccccc8c8ccccc78)cccc6c45)n3)cc2)cc1]

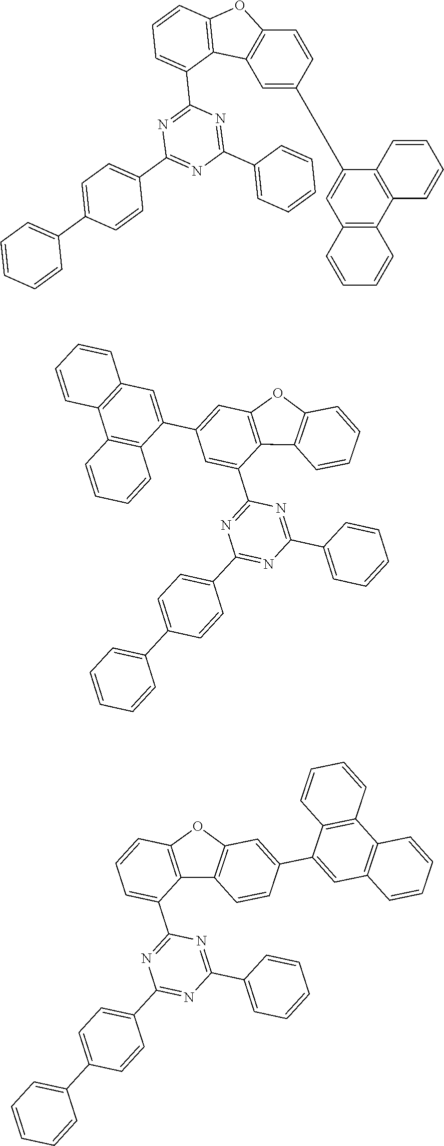 [SMILES: c1ccc(-c2ccc(-c3nc(-c4ccccc4)nc(-c4cccc5oc6ccc(-c7cc8ccccc8c8ccccc78)cc6c45)n3)cc2)cc1 ; c1ccc(-c2ccc(-c3nc(-c4ccccc4)nc(-c4cc(-c5cc6ccccc6c6ccccc56)cc5oc6ccccc6c45)n3)cc2)cc1 ; c1ccc(-c2ccc(-c3nc(-c4ccccc4)nc(-c4cccc5oc6cc(-c7cc8ccccc8c8ccccc78)ccc6c45)n3)cc2)cc1]

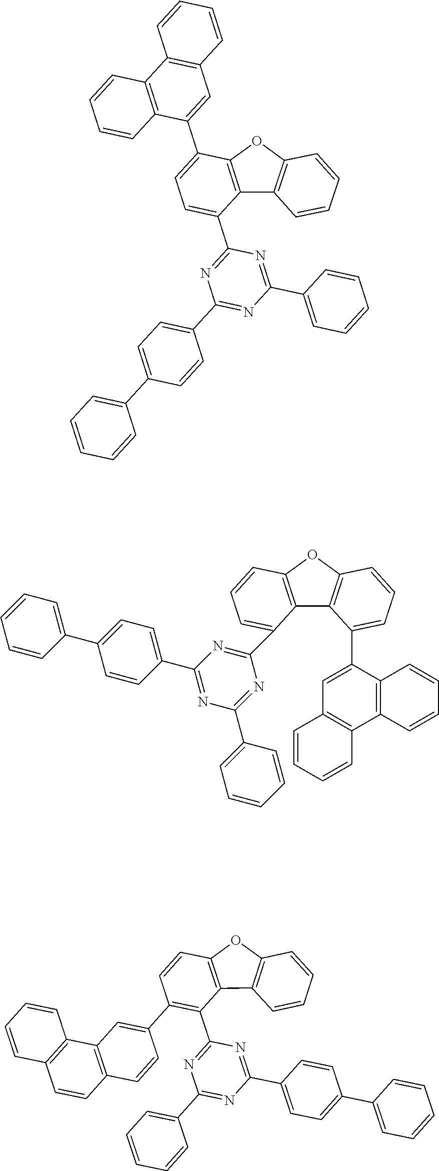 [SMILES: c1ccc(-c2ccc(-c3nc(-c4ccccc4)nc(-c4ccc(-c5cc6ccccc6c6ccccc56)c5oc6ccccc6c45)n3)cc2)cc1 ; c1ccc(-c2ccc(-c3nc(-c4ccccc4)nc(-c4cccc5oc6cccc(-c7cc8ccccc8c8ccccc78)c6c45)n3)cc2)cc1 ; c1ccc(-c2ccc(-c3nc(-c4ccccc4)nc(-c4c(-c5ccc6ccc7ccccc7c6c5)ccc5oc6ccccc6c45)n3)cc2)cc1]

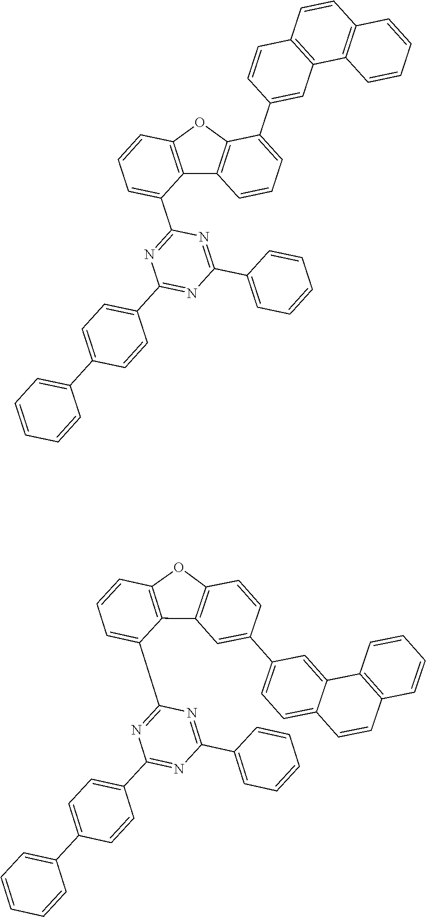 [SMILES: c1ccc(-c2ccc(-c3nc(-c4ccccc4)nc(-c4cccc5oc6c(-c7ccc8ccc9ccccc9c8c7)cccc6c45)n3)cc2)cc1 ; c1ccc(-c2ccc(-c3nc(-c4ccccc4)nc(-c4cccc5oc6ccc(-c7ccc8ccc9ccccc9c8c7)cc6c45)n3)cc2)cc1]

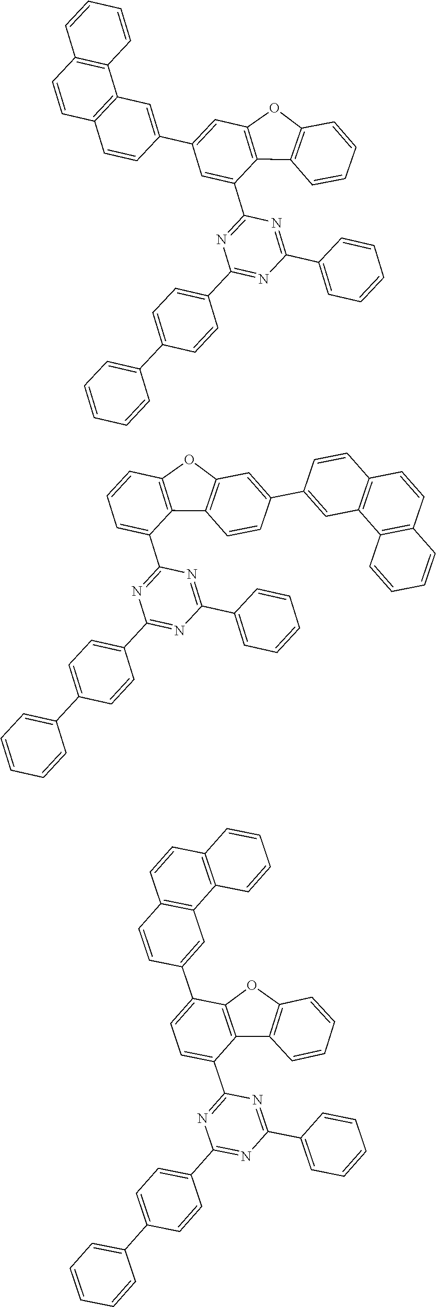

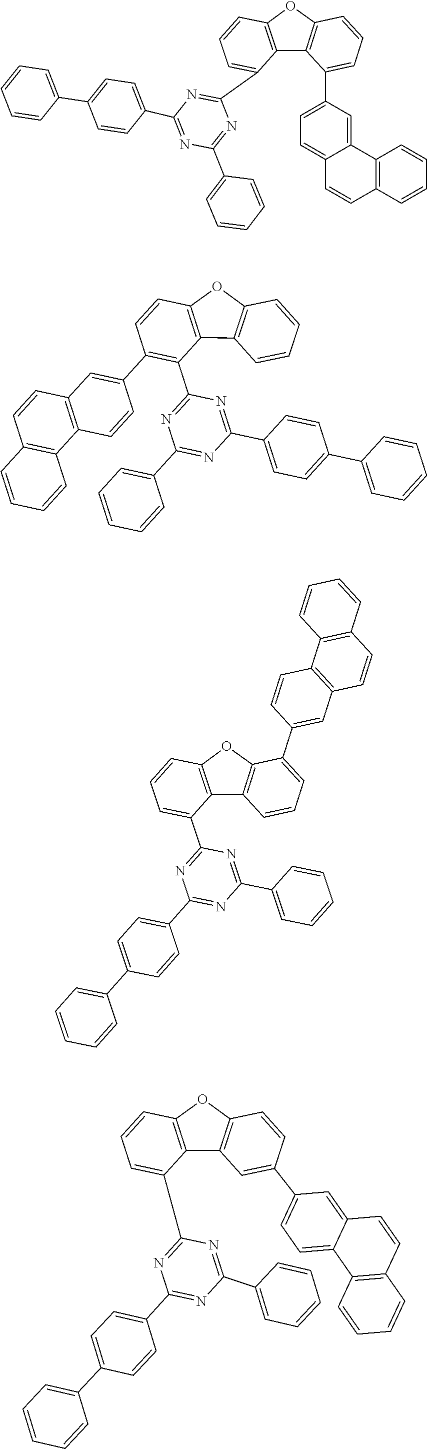 [SMILES: c1ccc(-c2ccc(-c3nc(-c4ccccc4)nc(-c4cccc5oc6cccc(-c7ccc8ccc9ccccc9c8c7)c6c45)n3)cc2)cc1 ; c1ccc(-c2ccc(-c3nc(-c4ccccc4)nc(-c4c(-c5ccc6c(ccc7ccccc76)c5)ccc5oc6ccccc6c45)n3)cc2)cc1 ; c1ccc(-c2ccc(-c3nc(-c4ccccc4)nc(-c4cccc5oc6c(-c7ccc8c(ccc9ccccc98)c7)cccc6c45)n3)cc2)cc1 ; c1ccc(-c2ccc(-c3nc(-c4ccccc4)nc(-c4cccc5oc6ccc(-c7ccc8c(ccc9ccccc98)c7)cc6c45)n3)cc2)cc1]

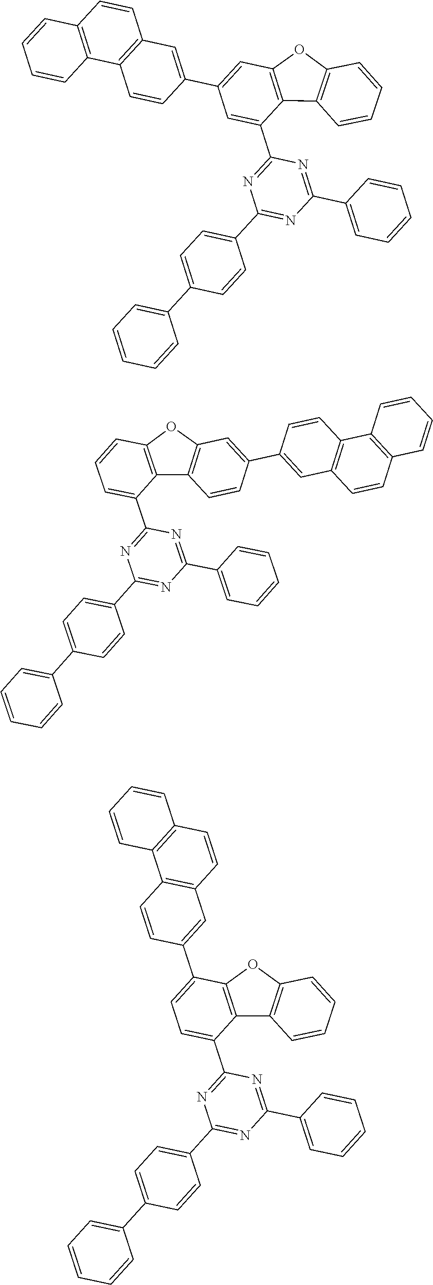 [SMILES: c1ccc(-c2ccc(-c3nc(-c4ccccc4)nc(-c4cc(-c5ccc6c(ccc7ccccc76)c5)cc5oc6ccccc6c45)n3)cc2)cc1 ; c1ccc(-c2ccc(-c3nc(-c4ccccc4)nc(-c4cccc5oc6cc(-c7ccc8c(ccc9ccccc98)c7)ccc6c45)n3)cc2)cc1 ; c1ccc(-c2ccc(-c3nc(-c4ccccc4)nc(-c4ccc(-c5ccc6c(ccc7ccccc76)c5)c5oc6ccccc6c45)n3)cc2)cc1]

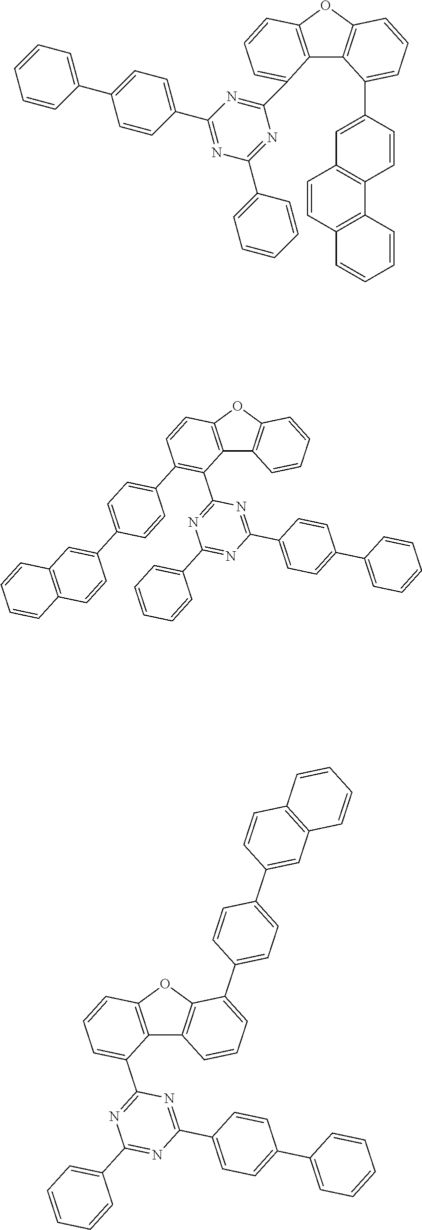 [SMILES: c1ccc(-c2ccc(-c3nc(-c4ccccc4)nc(-c4cccc5oc6cccc(-c7ccc8c(ccc9ccccc98)c7)c6c45)n3)cc2)cc1 ; c1ccc(-c2ccc(-c3nc(-c4ccccc4)nc(-c4c(-c5ccc(-c6ccc7ccccc7c6)cc5)ccc5oc6ccccc6c45)n3)cc2)cc1 ; c1ccc(-c2ccc(-c3nc(-c4ccccc4)nc(-c4cccc5oc6c(-c7ccc(-c8ccc9ccccc9c8)cc7)cccc6c45)n3)cc2)cc1]

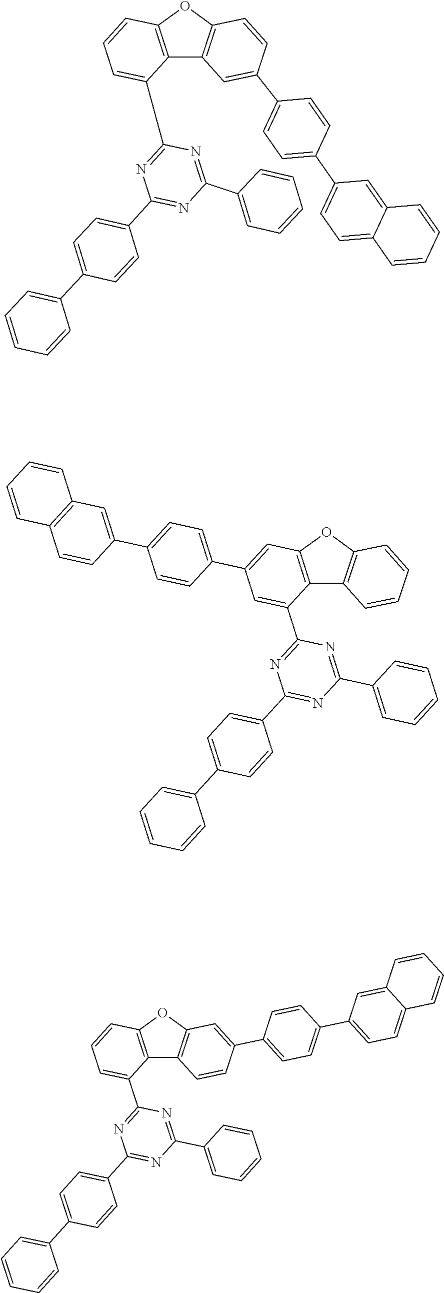 [SMILES: c1ccc(-c2ccc(-c3nc(-c4ccccc4)nc(-c4cccc5oc6ccc(-c7ccc(-c8ccc9ccccc9c8)cc7)cc6c45)n3)cc2)cc1 ; c1ccc(-c2ccc(-c3nc(-c4ccccc4)nc(-c4cc(-c5ccc(-c6ccc7ccccc7c6)cc5)cc5oc6ccccc6c45)n3)cc2)cc1 ; c1ccc(-c2ccc(-c3nc(-c4ccccc4)nc(-c4cccc5oc6cc(-c7ccc(-c8ccc9ccccc9c8)cc7)ccc6c45)n3)cc2)cc1]

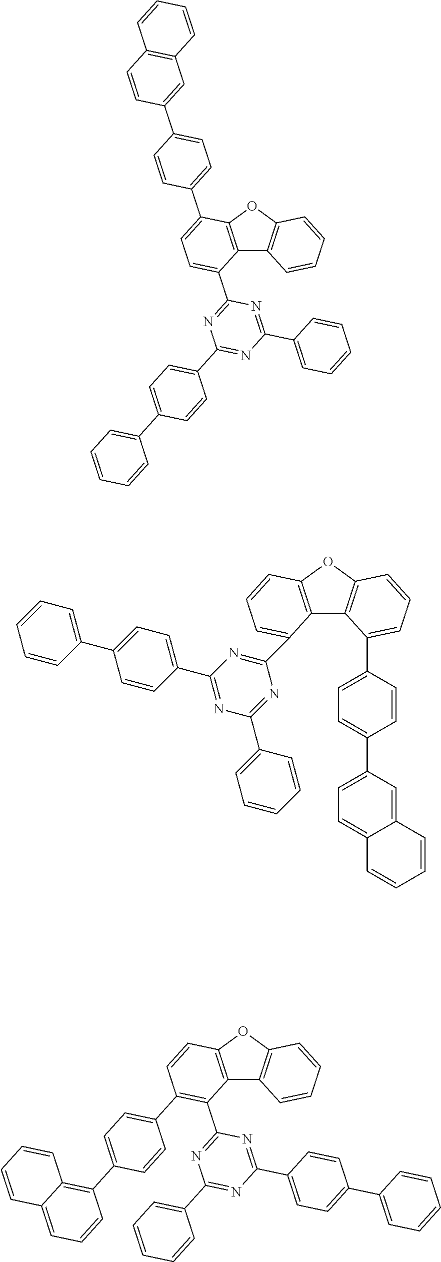 [SMILES: c1ccc(-c2ccc(-c3nc(-c4ccccc4)nc(-c4ccc(-c5ccc(-c6ccc7ccccc7c6)cc5)c5oc6ccccc6c45)n3)cc2)cc1 ; c1ccc(-c2ccc(-c3nc(-c4ccccc4)nc(-c4cccc5oc6cccc(-c7ccc(-c8ccc9ccccc9c8)cc7)c6c45)n3)cc2)cc1 ; c1ccc(-c2ccc(-c3nc(-c4ccccc4)nc(-c4c(-c5ccc(-c6cccc7ccccc67)cc5)ccc5oc6ccccc6c45)n3)cc2)cc1]

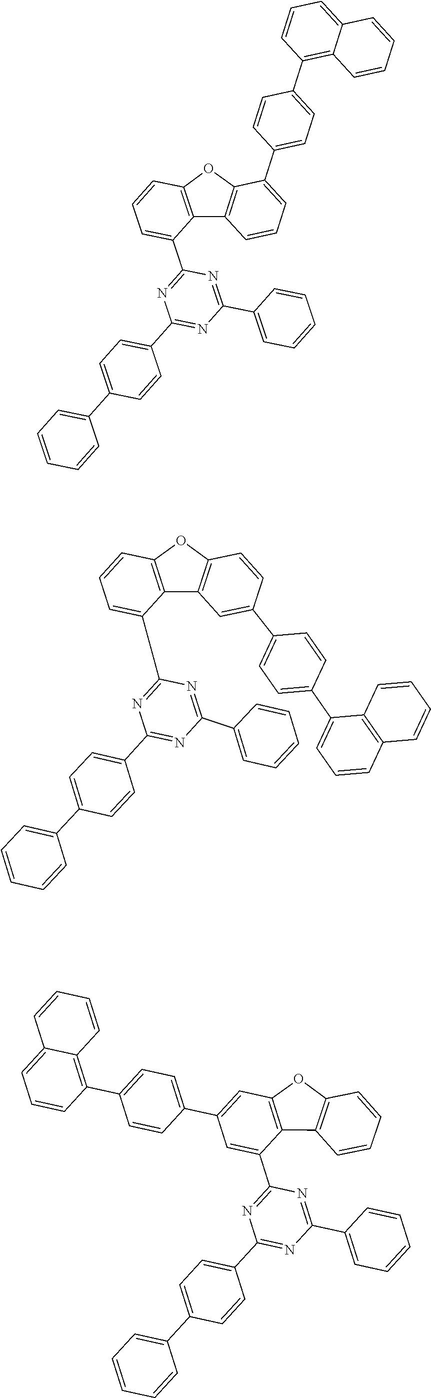 [SMILES: c1ccc(-c2ccc(-c3nc(-c4ccccc4)nc(-c4cccc5oc6c(-c7ccc(-c8cccc9ccccc89)cc7)cccc6c45)n3)cc2)cc1 ; c1ccc(-c2ccc(-c3nc(-c4ccccc4)nc(-c4cccc5oc6ccc(-c7ccc(-c8cccc9ccccc89)cc7)cc6c45)n3)cc2)cc1 ; c1ccc(-c2ccc(-c3nc(-c4ccccc4)nc(-c4cc(-c5ccc(-c6cccc7ccccc67)cc5)cc5oc6ccccc6c45)n3)cc2)cc1]

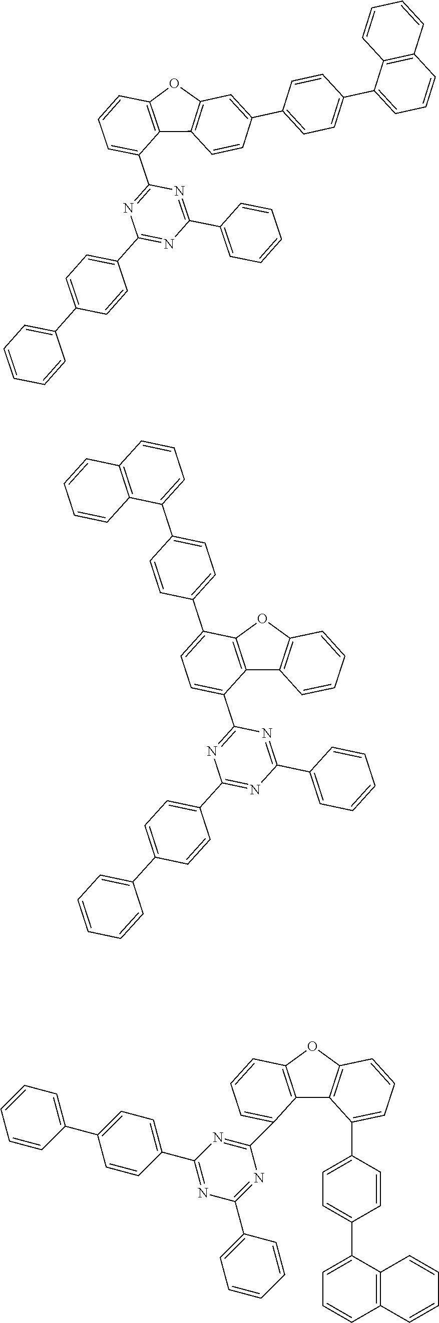 [SMILES: c1ccc(-c2ccc(-c3nc(-c4ccccc4)nc(-c4cccc5oc6cc(-c7ccc(-c8cccc9ccccc89)cc7)ccc6c45)n3)cc2)cc1 ; c1ccc(-c2ccc(-c3nc(-c4ccccc4)nc(-c4ccc(-c5ccc(-c6cccc7ccccc67)cc5)c5oc6ccccc6c45)n3)cc2)cc1 ; c1ccc(-c2ccc(-c3nc(-c4ccccc4)nc(-c4cccc5oc6cccc(-c7ccc(-c8cccc9ccccc89)cc7)c6c45)n3)cc2)cc1]

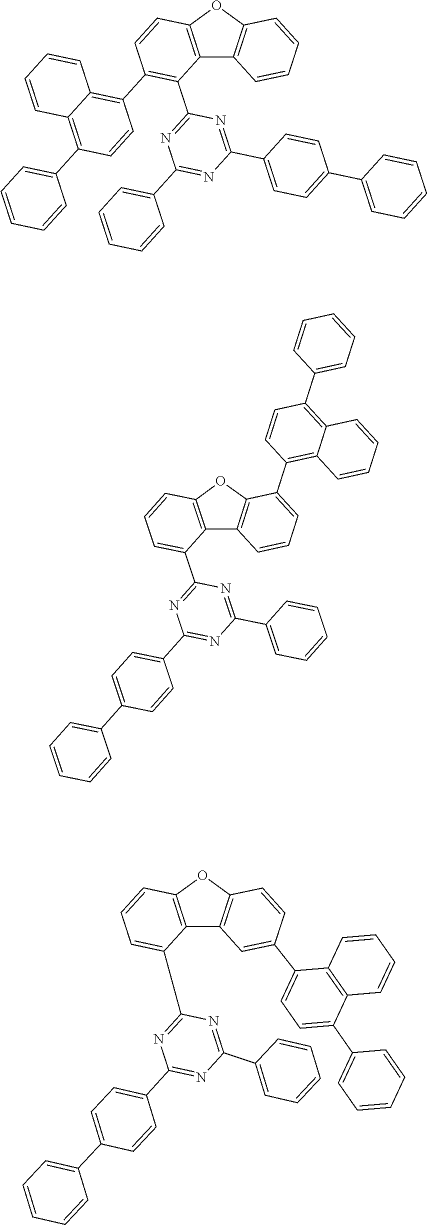 [SMILES: c1ccc(-c2ccc(-c3nc(-c4ccccc4)nc(-c4c(-c5ccc(-c6ccccc6)c6ccccc56)ccc5oc6ccccc6c45)n3)cc2)cc1 ; c1ccc(-c2ccc(-c3nc(-c4ccccc4)nc(-c4cccc5oc6c(-c7ccc(-c8ccccc8)c8ccccc78)cccc6c45)n3)cc2)cc1 ; c1ccc(-c2ccc(-c3nc(-c4ccccc4)nc(-c4cccc5oc6ccc(-c7ccc(-c8ccccc8)c8ccccc78)cc6c45)n3)cc2)cc1]

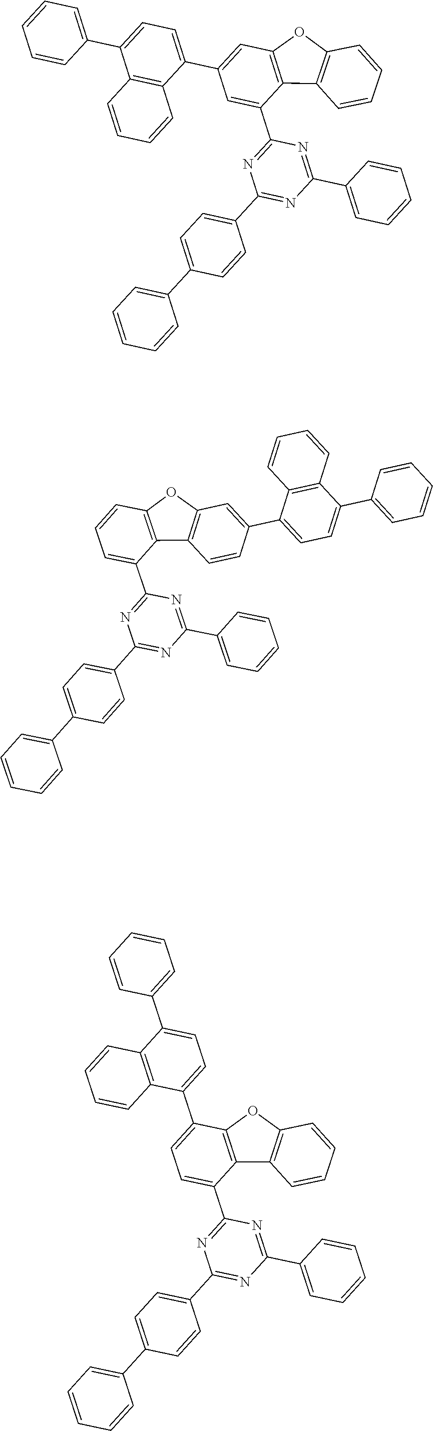 [SMILES: c1ccc(-c2ccc(-c3nc(-c4ccccc4)nc(-c4cc(-c5ccc(-c6ccccc6)c6ccccc56)cc5oc6ccccc6c45)n3)cc2)cc1 ; c1ccc(-c2ccc(-c3nc(-c4ccccc4)nc(-c4cccc5oc6cc(-c7ccc(-c8ccccc8)c8ccccc78)ccc6c45)n3)cc2)cc1 ; c1ccc(-c2ccc(-c3nc(-c4ccccc4)nc(-c4ccc(-c5ccc(-c6ccccc6)c6ccccc56)c5oc6ccccc6c45)n3)cc2)cc1]

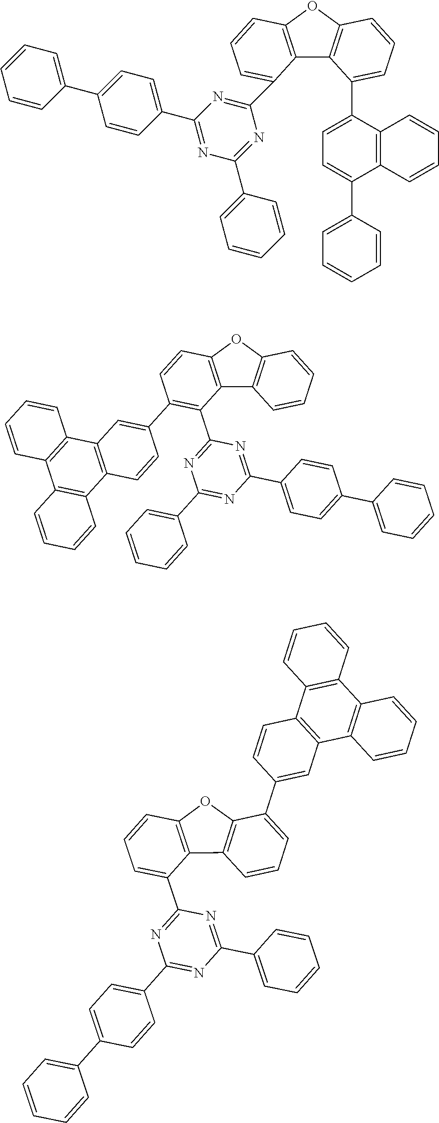 [SMILES: c1ccc(-c2ccc(-c3nc(-c4ccccc4)nc(-c4cccc5oc6cccc(-c7ccc(-c8ccccc8)c8ccccc78)c6c45)n3)cc2)cc1 ; c1ccc(-c2ccc(-c3nc(-c4ccccc4)nc(-c4c(-c5ccc6c7ccccc7c7ccccc7c6c5)ccc5oc6ccccc6c45)n3)cc2)cc1 ; c1ccc(-c2ccc(-c3nc(-c4ccccc4)nc(-c4cccc5oc6c(-c7ccc8c9ccccc9c9ccccc9c8c7)cccc6c45)n3)cc2)cc1]

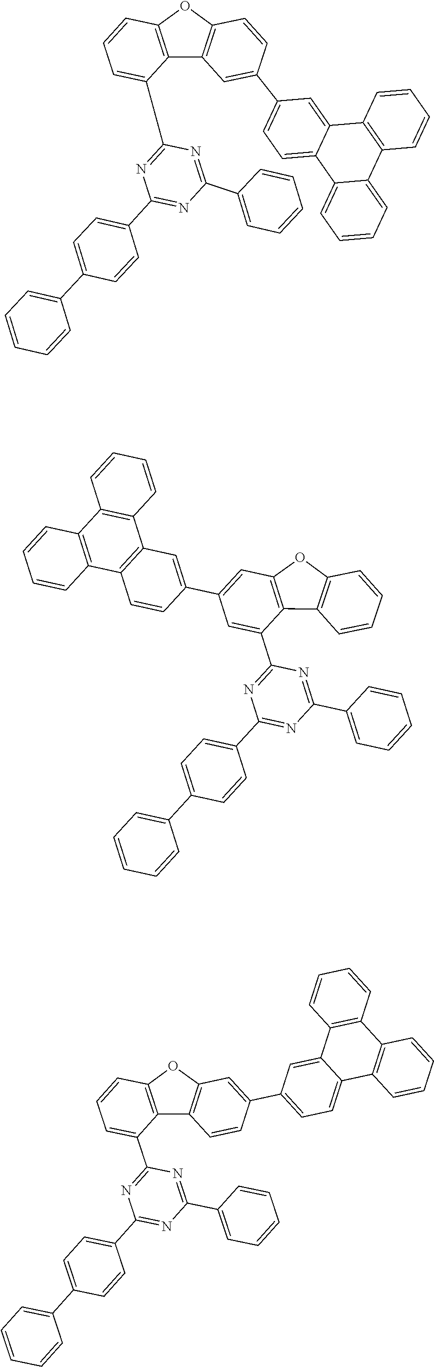 [SMILES: c1ccc(-c2ccc(-c3nc(-c4ccccc4)nc(-c4cccc5oc6ccc(-c7ccc8c9ccccc9c9ccccc9c8c7)cc6c45)n3)cc2)cc1 ; c1ccc(-c2ccc(-c3nc(-c4ccccc4)nc(-c4cc(-c5ccc6c7ccccc7c7ccccc7c6c5)cc5oc6ccccc6c45)n3)cc2)cc1 ; c1ccc(-c2ccc(-c3nc(-c4ccccc4)nc(-c4cccc5oc6cc(-c7ccc8c9ccccc9c9ccccc9c8c7)ccc6c45)n3)cc2)cc1]

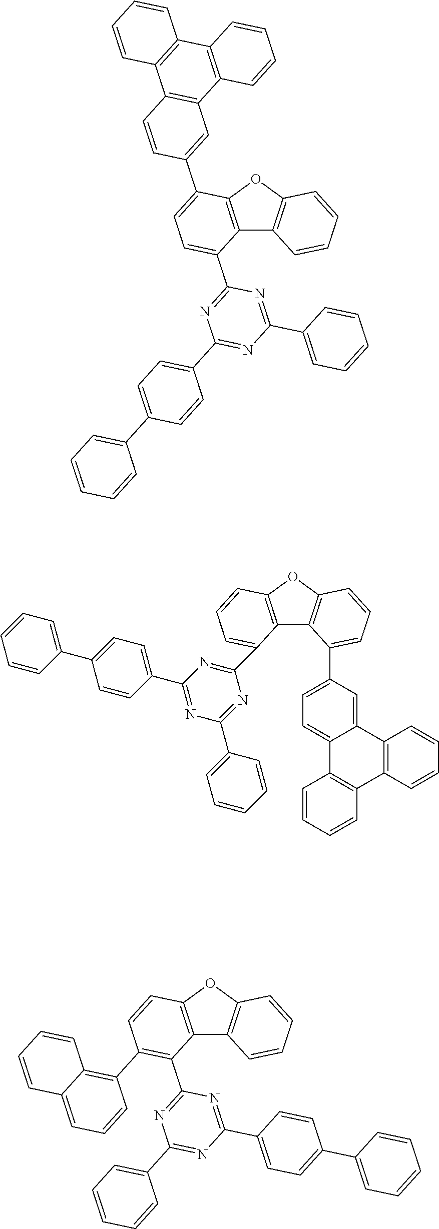 [SMILES: c1ccc(-c2ccc(-c3nc(-c4ccccc4)nc(-c4ccc(-c5ccc6c7ccccc7c7ccccc7c6c5)c5oc6ccccc6c45)n3)cc2)cc1 ; c1ccc(-c2ccc(-c3nc(-c4ccccc4)nc(-c4cccc5oc6cccc(-c7ccc8c9ccccc9c9ccccc9c8c7)c6c45)n3)cc2)cc1 ; c1ccc(-c2ccc(-c3nc(-c4ccccc4)nc(-c4c(-c5cccc6ccccc56)ccc5oc6ccccc6c45)n3)cc2)cc1]

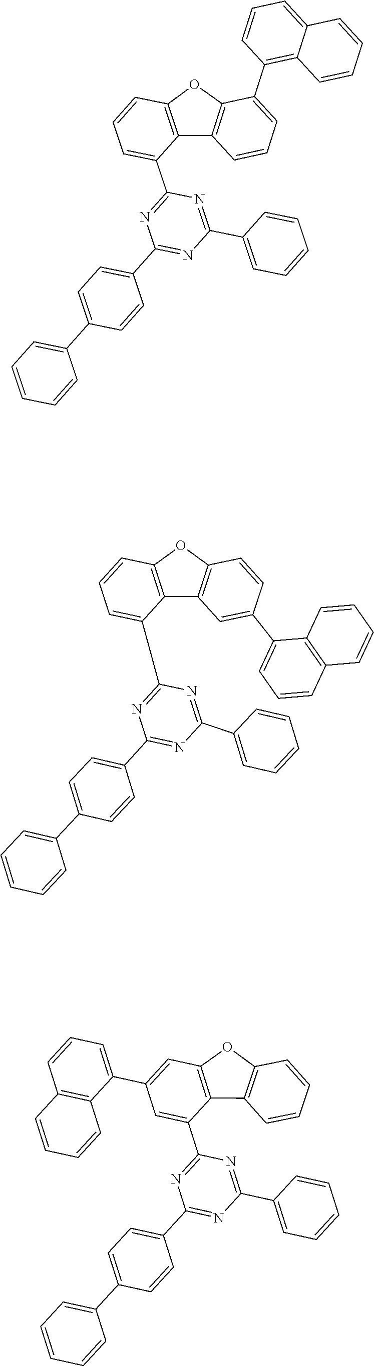 [SMILES: c1ccc(-c2ccc(-c3nc(-c4ccccc4)nc(-c4cccc5oc6c(-c7cccc8ccccc78)cccc6c45)n3)cc2)cc1 ; c1ccc(-c2ccc(-c3nc(-c4ccccc4)nc(-c4cccc5oc6ccc(-c7cccc8ccccc78)cc6c45)n3)cc2)cc1 ; c1ccc(-c2ccc(-c3nc(-c4ccccc4)nc(-c4cc(-c5cccc6ccccc56)cc5oc6ccccc6c45)n3)cc2)cc1]

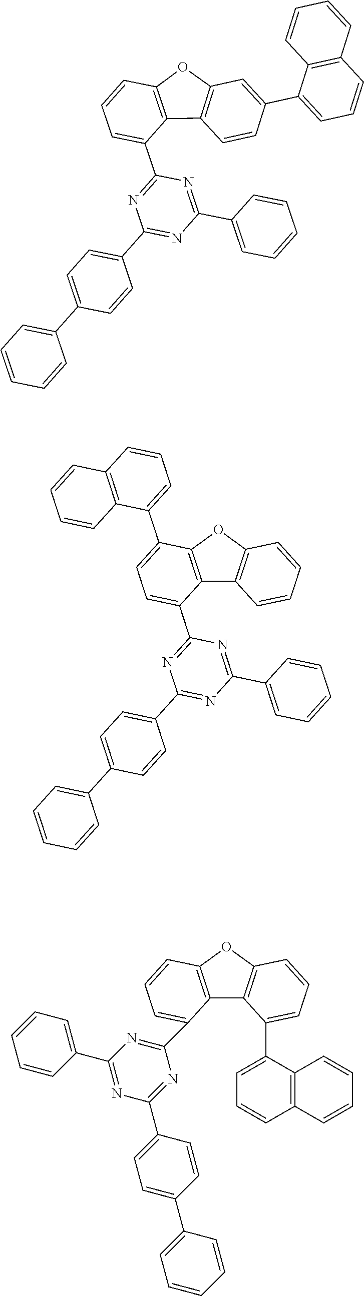 [SMILES: c1ccc(-c2ccc(-c3nc(-c4ccccc4)nc(-c4cccc5oc6cc(-c7cccc8ccccc78)ccc6c45)n3)cc2)cc1 ; c1ccc(-c2ccc(-c3nc(-c4ccccc4)nc(-c4ccc(-c5cccc6ccccc56)c5oc6ccccc6c45)n3)cc2)cc1 ; c1ccc(-c2ccc(-c3nc(-c4ccccc4)nc(-c4cccc5oc6cccc(-c7cccc8ccccc78)c6c45)n3)cc2)cc1]

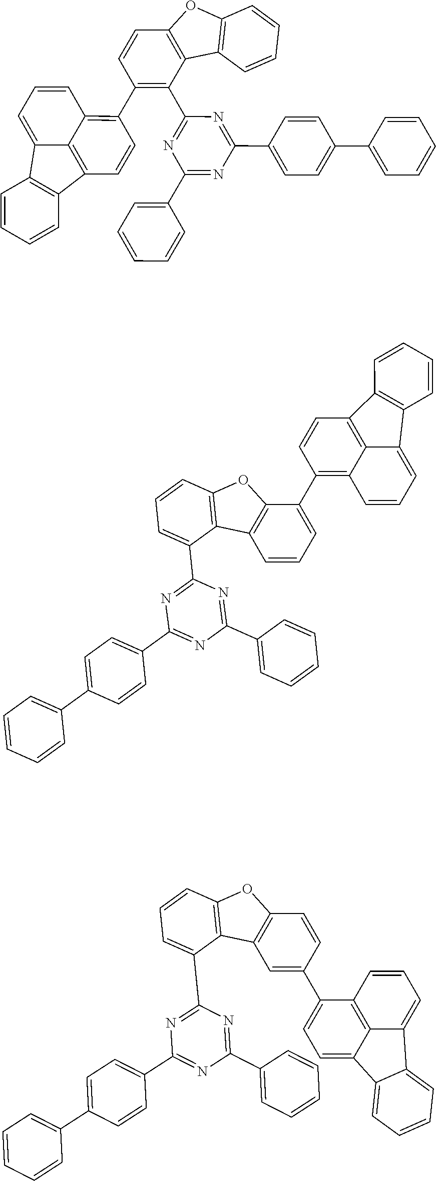 [SMILES: c1ccc(-c2ccc(-c3nc(-c4ccccc4)nc(-c4c(-c5ccc6c7c(cccc57)-c5ccccc5-6)ccc5oc6ccccc6c45)n3)cc2)cc1 ; c1ccc(-c2ccc(-c3nc(-c4ccccc4)nc(-c4cccc5oc6c(-c7ccc8c9c(cccc79)-c7ccccc7-8)cccc6c45)n3)cc2)cc1 ; c1ccc(-c2ccc(-c3nc(-c4ccccc4)nc(-c4cccc5oc6ccc(-c7ccc8c9c(cccc79)-c7ccccc7-8)cc6c45)n3)cc2)cc1]

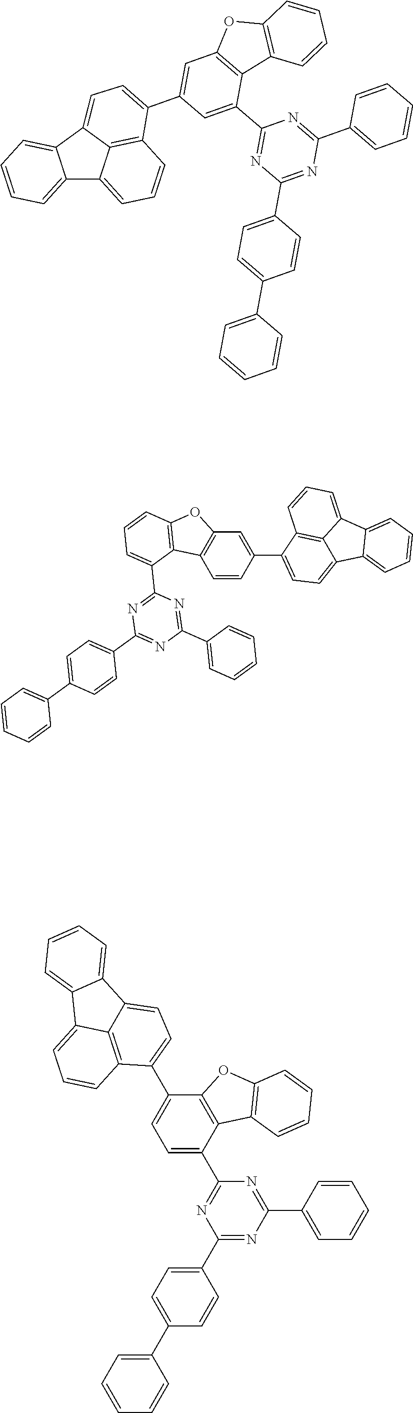 [SMILES: c1ccc(-c2ccc(-c3nc(-c4ccccc4)nc(-c4cc(-c5ccc6c7c(cccc57)-c5ccccc5-6)cc5oc6ccccc6c45)n3)cc2)cc1 ; c1ccc(-c2ccc(-c3nc(-c4ccccc4)nc(-c4cccc5oc6cc(-c7ccc8c9c(cccc79)-c7ccccc7-8)ccc6c45)n3)cc2)cc1 ; c1ccc(-c2ccc(-c3nc(-c4ccccc4)nc(-c4ccc(-c5ccc6c7c(cccc57)-c5ccccc5-6)c5oc6ccccc6c45)n3)cc2)cc1]

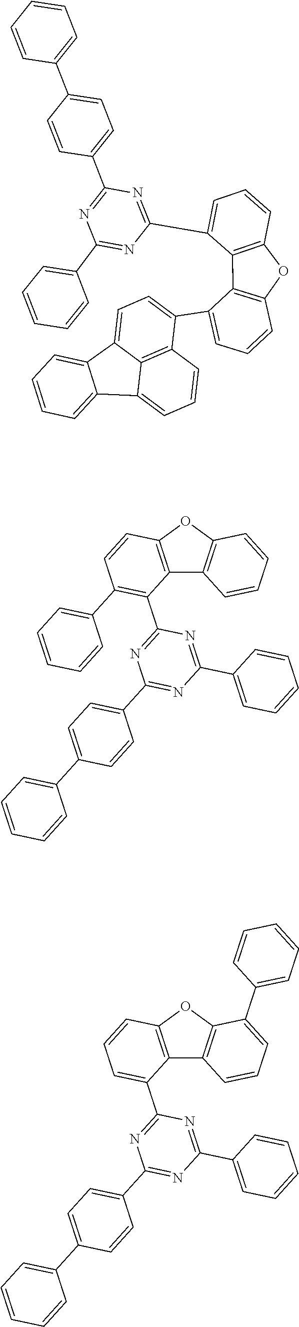 [SMILES: c1ccc(-c2ccc(-c3nc(-c4ccccc4)nc(-c4cccc5oc6cccc(-c7ccc8c9c(cccc79)-c7ccccc7-8)c6c45)n3)cc2)cc1 ; c1ccc(-c2ccc(-c3nc(-c4ccccc4)nc(-c4c(-c5ccccc5)ccc5oc6ccccc6c45)n3)cc2)cc1 ; c1ccc(-c2ccc(-c3nc(-c4ccccc4)nc(-c4cccc5oc6c(-c7ccccc7)cccc6c45)n3)cc2)cc1]

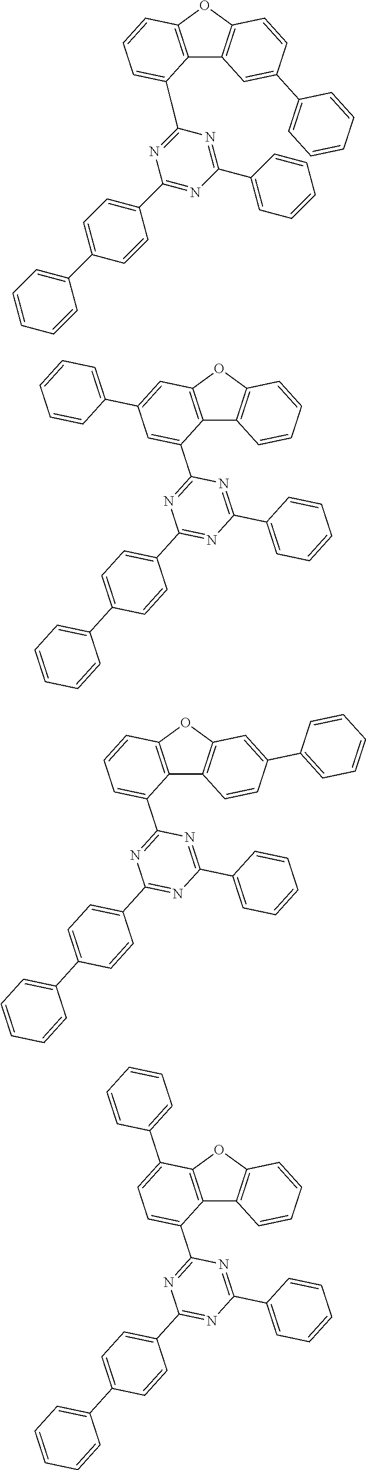

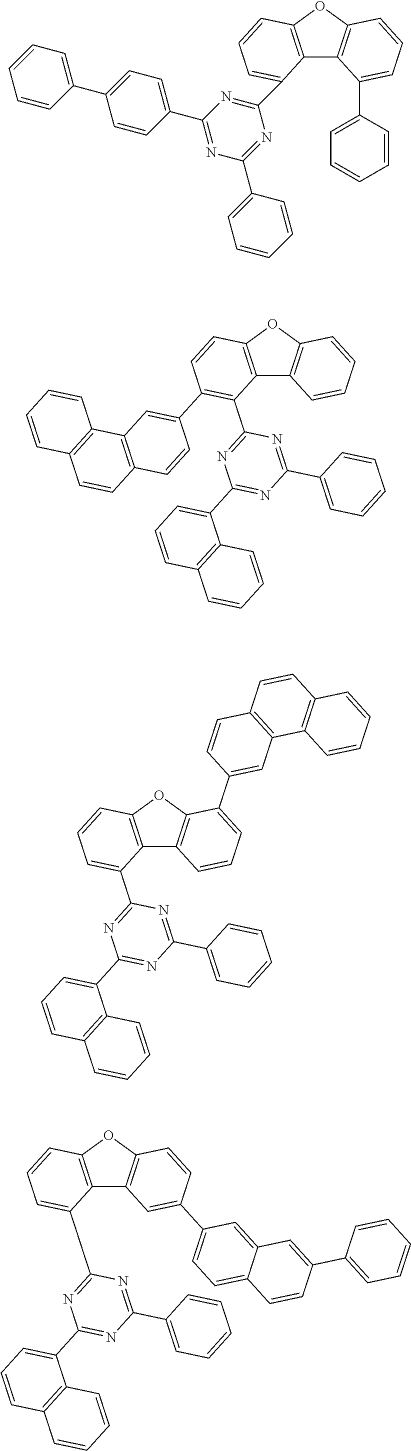 [SMILES: c1ccc(-c2ccc(-c3nc(-c4ccccc4)nc(-c4cccc5oc6cccc(-c7ccccc7)c6c45)n3)cc2)cc1 ; c1ccc(-c2nc(-c3cccc4ccccc34)nc(-c3c(-c4ccc5ccc6ccccc6c5c4)ccc4oc5ccccc5c34)n2)cc1 ; c1ccc(-c2nc(-c3cccc4ccccc34)nc(-c3cccc4oc5c(-c6ccc7ccc8ccccc8c7c6)cccc5c34)n2)cc1 ; c1ccc(-c2ccc3ccc(-c4ccc5oc6cccc(-c7nc(-c8ccccc8)nc(-c8cccc9ccccc89)n7)c6c5c4)cc3c2)cc1]

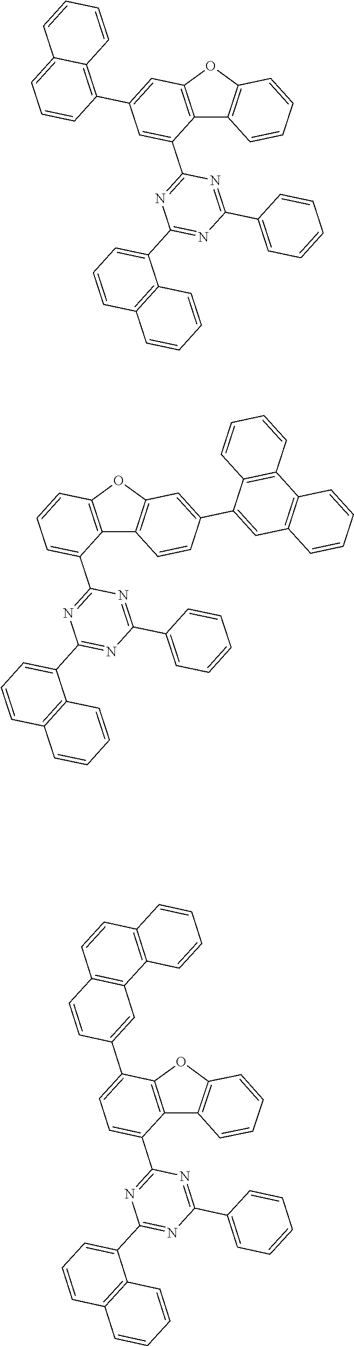 [SMILES: c1ccc(-c2nc(-c3cccc4ccccc34)nc(-c3cc(-c4cccc5ccccc45)cc4oc5ccccc5c34)n2)cc1 ; c1ccc(-c2nc(-c3cccc4ccccc34)nc(-c3cccc4oc5cc(-c6cc7ccccc7c7ccccc67)ccc5c34)n2)cc1 ; c1ccc(-c2nc(-c3cccc4ccccc34)nc(-c3ccc(-c4ccc5ccc6ccccc6c5c4)c4oc5ccccc5c34)n2)cc1]

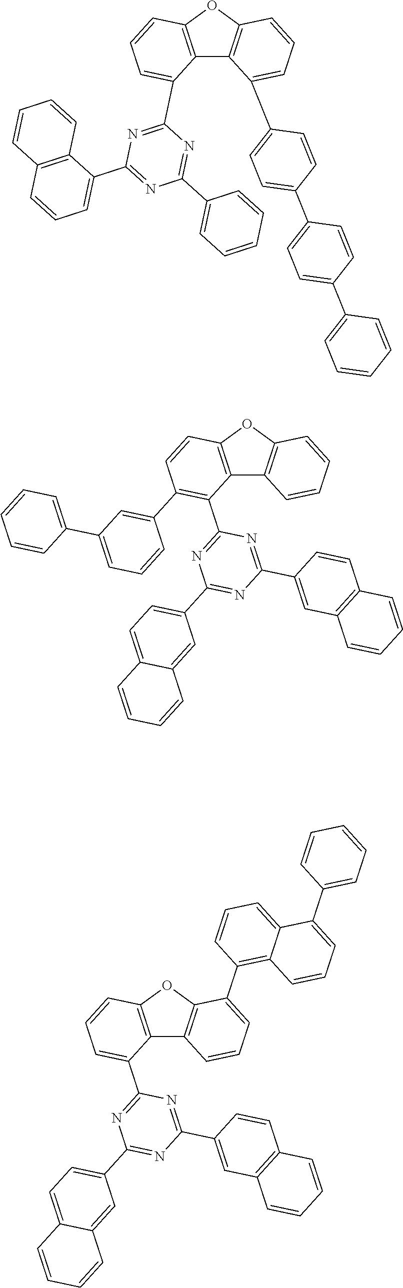 [SMILES: c1ccc(-c2ccc(-c3ccc(-c4cccc5oc6cccc(-c7nc(-c8ccccc8)nc(-c8cccc9ccccc89)n7)c6c45)cc3)cc2)cc1 ; c1ccc(-c2cccc(-c3ccc4oc5ccccc5c4c3-c3nc(-c4ccc5ccccc5c4)nc(-c4ccc5ccccc5c4)n3)c2)cc1 ; c1ccc(-c2cccc3c(-c4cccc5c4oc4cccc(-c6nc(-c7ccc8ccccc8c7)nc(-c7ccc8ccccc8c7)n6)c45)cccc23)cc1]

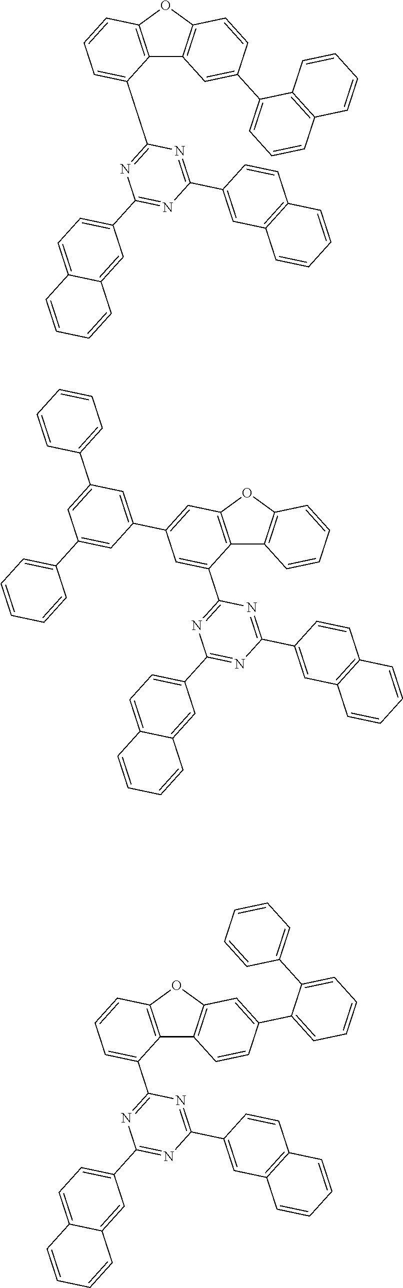 [SMILES: c1ccc2cc(-c3nc(-c4ccc5ccccc5c4)nc(-c4cccc5oc6ccc(-c7cccc8ccccc78)cc6c45)n3)ccc2c1 ; c1ccc(-c2cc(-c3ccccc3)cc(-c3cc(-c4nc(-c5ccc6ccccc6c5)nc(-c5ccc6ccccc6c5)n4)c4c(c3)oc3ccccc34)c2)cc1 ; c1ccc(-c2ccccc2-c2ccc3c(c2)oc2cccc(-c4nc(-c5ccc6ccccc6c5)nc(-c5ccc6ccccc6c5)n4)c23)cc1]

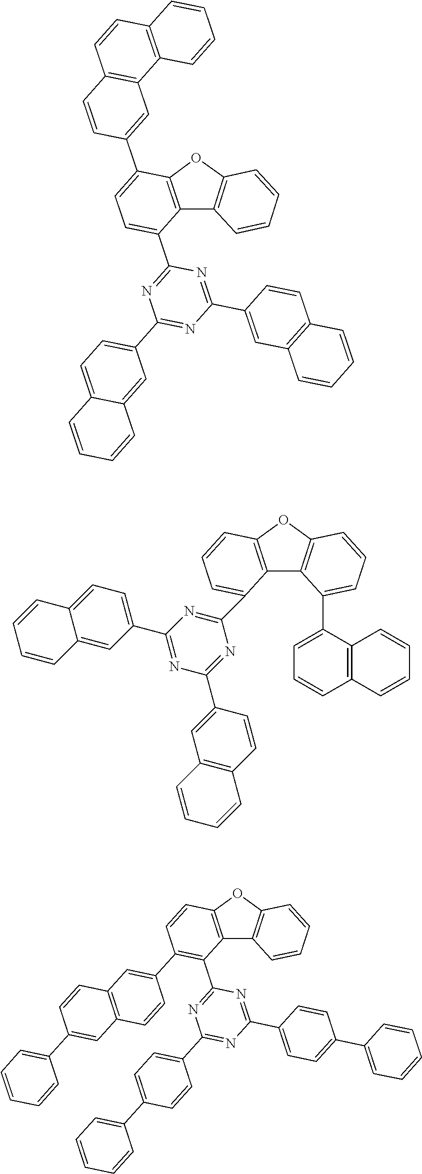 [SMILES: c1ccc2cc(-c3nc(-c4ccc5ccccc5c4)nc(-c4ccc(-c5ccc6ccc7ccccc7c6c5)c5oc6ccccc6c45)n3)ccc2c1 ; c1ccc2cc(-c3nc(-c4ccc5ccccc5c4)nc(-c4cccc5oc6cccc(-c7cccc8ccccc78)c6c45)n3)ccc2c1 ; c1ccc(-c2ccc(-c3nc(-c4ccc(-c5ccccc5)cc4)nc(-c4c(-c5ccc6cc(-c7ccccc7)ccc6c5)ccc5oc6ccccc6c45)n3)cc2)cc1]

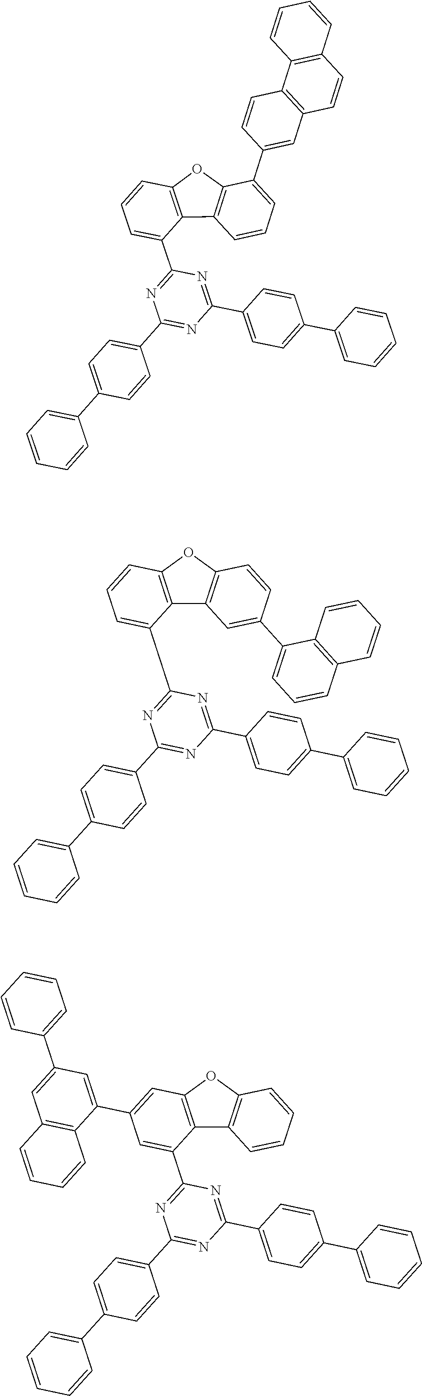 [SMILES: c1ccc(-c2ccc(-c3nc(-c4ccc(-c5ccccc5)cc4)nc(-c4cccc5oc6c(-c7ccc8c(ccc9ccccc98)c7)cccc6c45)n3)cc2)cc1 ; c1ccc(-c2ccc(-c3nc(-c4ccc(-c5ccccc5)cc4)nc(-c4cccc5oc6ccc(-c7cccc8ccccc78)cc6c45)n3)cc2)cc1 ; c1ccc(-c2ccc(-c3nc(-c4ccc(-c5ccccc5)cc4)nc(-c4cc(-c5cc(-c6ccccc6)cc6ccccc56)cc5oc6ccccc6c45)n3)cc2)cc1]

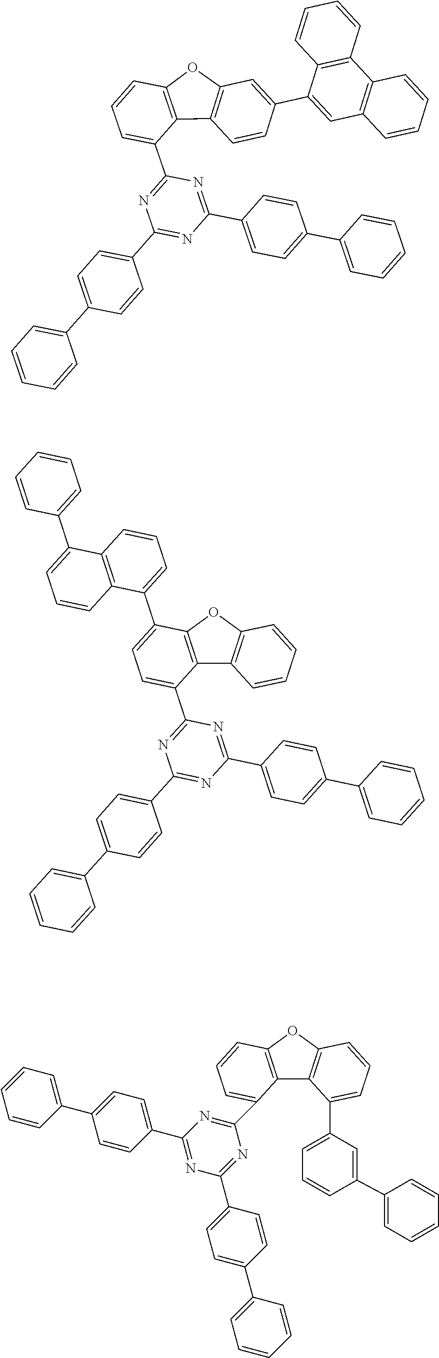 [SMILES: c1ccc(-c2ccc(-c3nc(-c4ccc(-c5ccccc5)cc4)nc(-c4cccc5oc6cc(-c7cc8ccccc8c8ccccc78)ccc6c45)n3)cc2)cc1 ; c1ccc(-c2ccc(-c3nc(-c4ccc(-c5ccccc5)cc4)nc(-c4ccc(-c5cccc6c(-c7ccccc7)cccc56)c5oc6ccccc6c45)n3)cc2)cc1 ; c1ccc(-c2ccc(-c3nc(-c4ccc(-c5ccccc5)cc4)nc(-c4cccc5oc6cccc(-c7cccc(-c8ccccc8)c7)c6c45)n3)cc2)cc1]

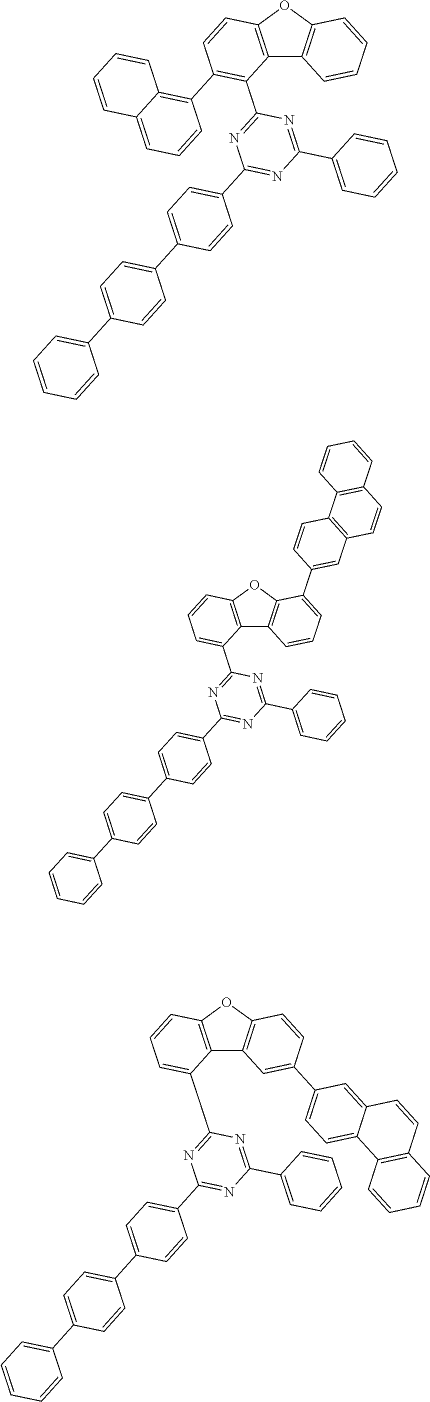 [SMILES: c1ccc(-c2ccc(-c3ccc(-c4nc(-c5ccccc5)nc(-c5c(-c6cccc7ccccc67)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9c(ccc%10ccccc%109)c8)cccc7c56)n4)cc3)cc2)cc1 ; c1ccc(-c2ccc(-c3ccc(-c4nc(-c5ccccc5)nc(-c5cccc6oc7ccc(-c8ccc9c(ccc%10ccccc%109)c8)cc7c56)n4)cc3)cc2)cc1]

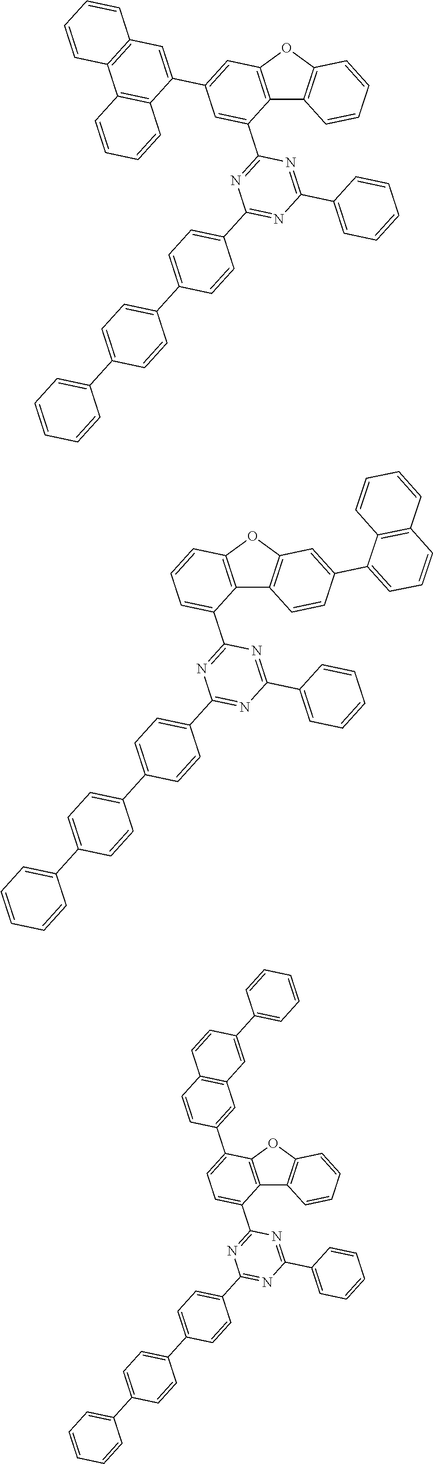 [SMILES: c1ccc(-c2ccc(-c3ccc(-c4nc(-c5ccccc5)nc(-c5cc(-c6cc7ccccc7c7ccccc67)cc6oc7ccccc7c56)n4)cc3)cc2)cc1 ; c1ccc(-c2ccc(-c3ccc(-c4nc(-c5ccccc5)nc(-c5cccc6oc7cc(-c8cccc9ccccc89)ccc7c56)n4)cc3)cc2)cc1 ; c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1]

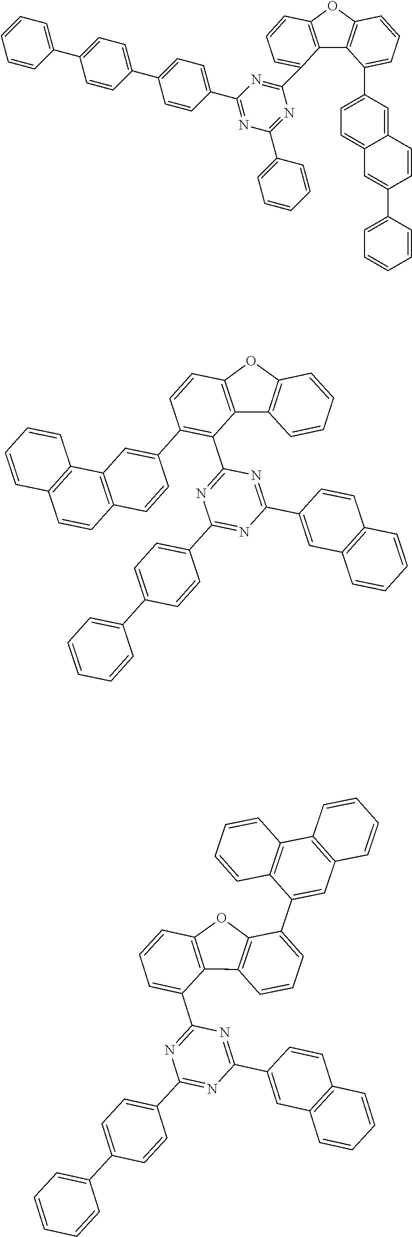 [SMILES: c1ccc(-c2ccc(-c3ccc(-c4nc(-c5ccccc5)nc(-c5cccc6oc7cccc(-c8ccc9cc(-c%10ccccc%10)ccc9c8)c7c56)n4)cc3)cc2)cc1 ; c1ccc(-c2ccc(-c3nc(-c4ccc5ccccc5c4)nc(-c4c(-c5ccc6ccc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8ccccc8c8ccccc78)cccc6c45)n3)cc2)cc1]

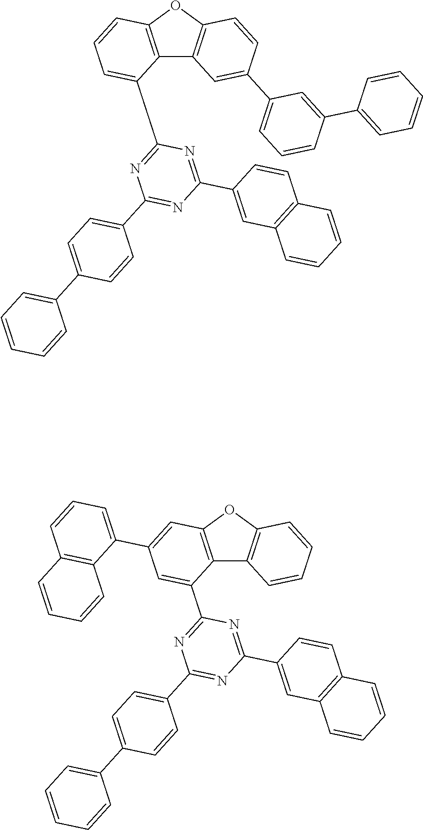 [SMILES: c1ccc(-c2ccc(-c3nc(-c4ccc5ccccc5c4)nc(-c4cccc5oc6ccc(-c7cccc(-c8ccccc8)c7)cc6c45)n3)cc2)cc1 ; c1ccc(-c2ccc(-c3nc(-c4ccc5ccccc5c4)nc(-c4cc(-c5cccc6ccccc56)cc5oc6ccccc6c45)n3)cc2)cc1]

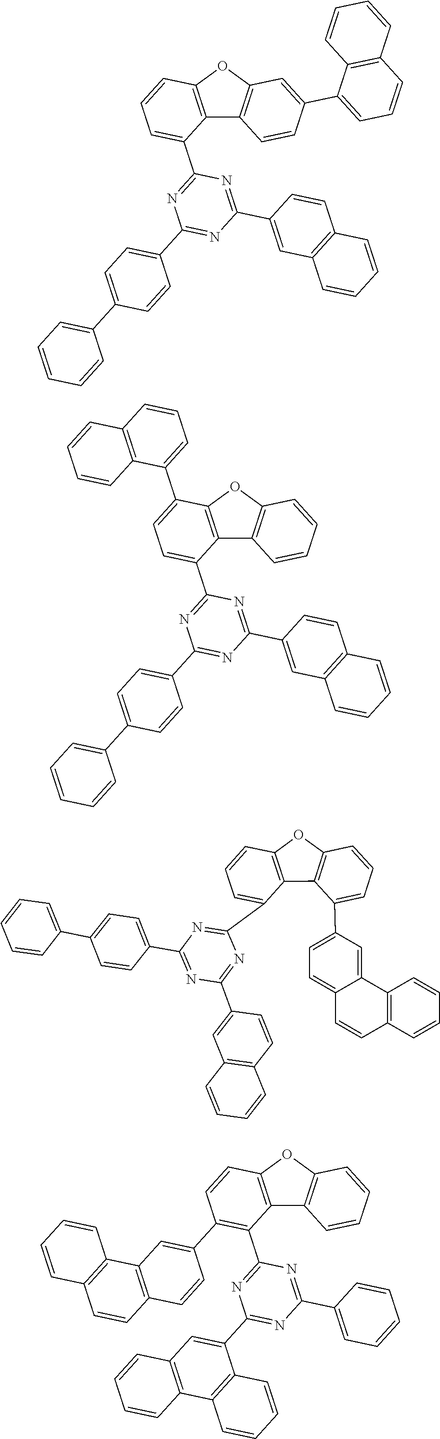

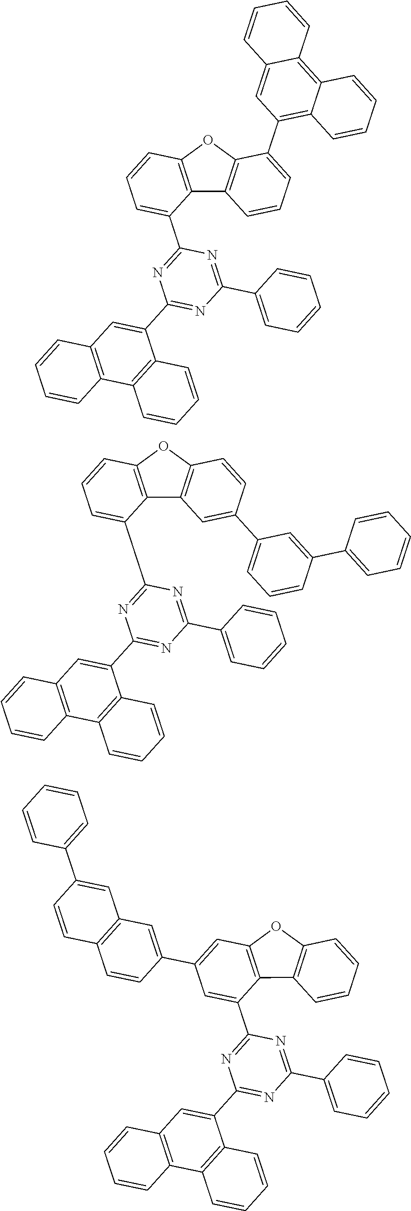

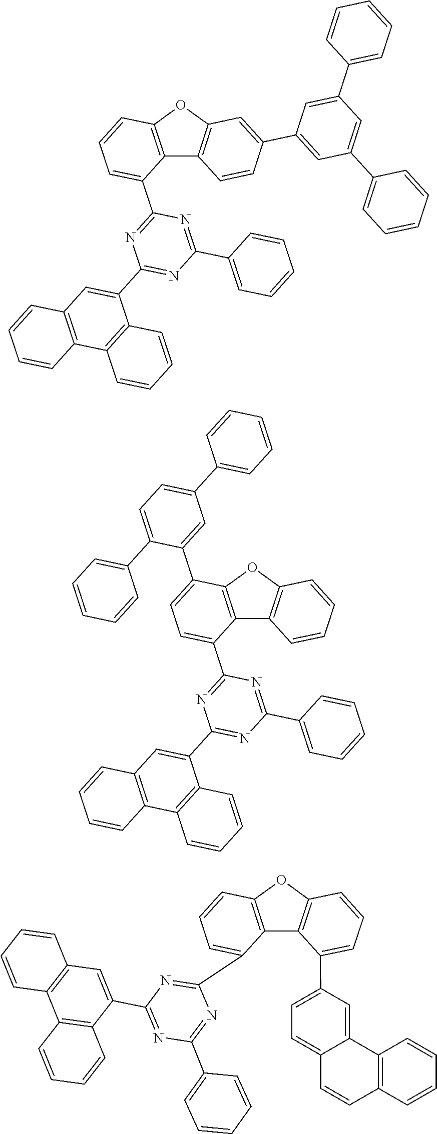

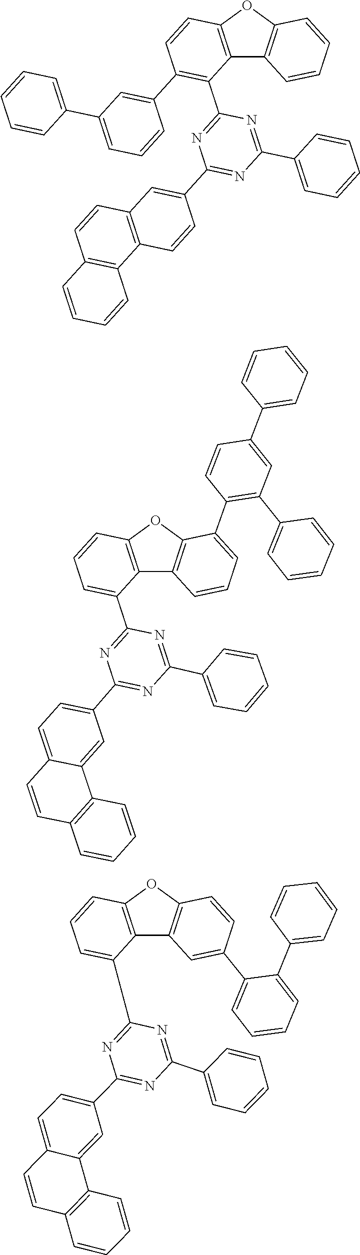

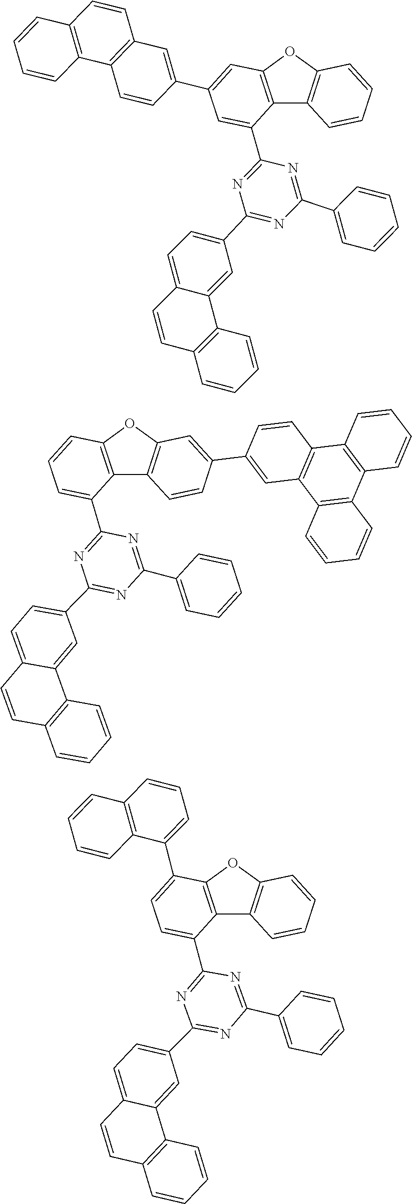

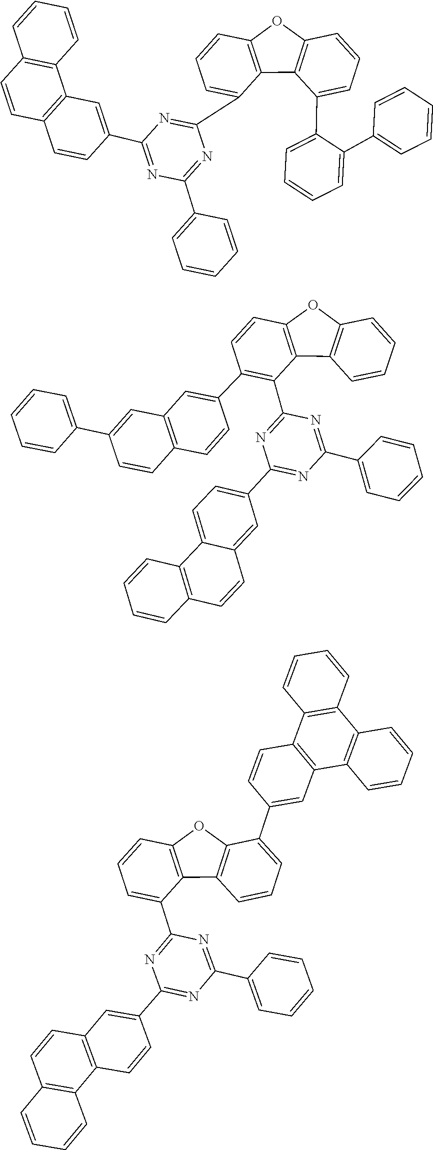 [SMILES: c1ccc(-c2nc(-c3ccc4ccc5ccccc5c4c3)nc(-c3cccc4oc5cccc(-c6ccccc6-c6ccccc6)c5c34)n2)cc1 ; c1ccc(-c2ccc3ccc(-c4ccc5oc6ccccc6c5c4-c4nc(-c5ccccc5)nc(-c5ccc6c(ccc7ccccc76)c5)n4)cc3c2)cc1 ; c1ccc(-c2nc(-c3ccc4c(ccc5ccccc54)c3)nc(-c3cccc4oc5c(-c6ccc7c8ccccc8c8ccccc8c7c6)cccc5c34)n2)cc1]

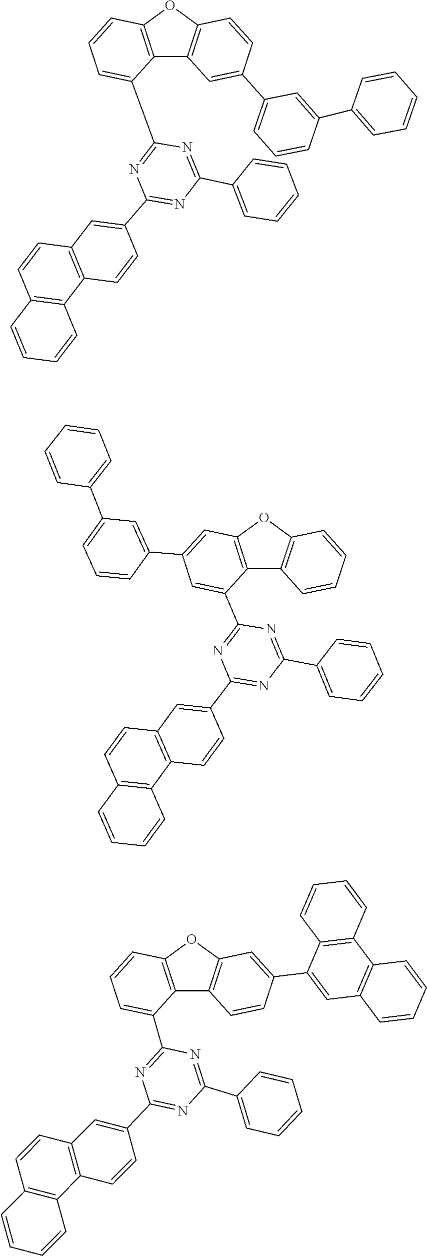 [SMILES: c1ccc(-c2cccc(-c3ccc4oc5cccc(-c6nc(-c7ccccc7)nc(-c7ccc8c(ccc9ccccc98)c7)n6)c5c4c3)c2)cc1 ; c1ccc(-c2cccc(-c3cc(-c4nc(-c5ccccc5)nc(-c5ccc6c(ccc7ccccc76)c5)n4)c4c(c3)oc3ccccc34)c2)cc1 ; c1ccc(-c2nc(-c3ccc4c(ccc5ccccc54)c3)nc(-c3cccc4oc5cc(-c6cc7ccccc7c7ccccc67)ccc5c34)n2)cc1]

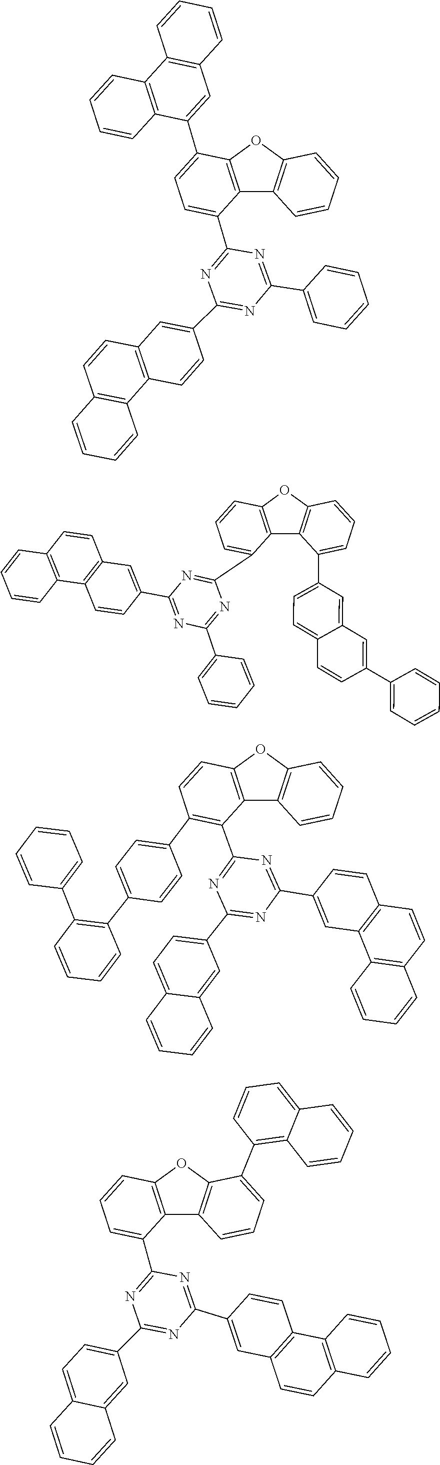 [SMILES: c1ccc(-c2nc(-c3ccc4c(ccc5ccccc54)c3)nc(-c3ccc(-c4cc5ccccc5c5ccccc45)c4oc5ccccc5c34)n2)cc1 ; c1ccc(-c2ccc3ccc(-c4cccc5oc6cccc(-c7nc(-c8ccccc8)nc(-c8ccc9c(ccc%10ccccc%109)c8)n7)c6c45)cc3c2)cc1 ; c1ccc(-c2ccccc2-c2ccc(-c3ccc4oc5ccccc5c4c3-c3nc(-c4ccc5ccccc5c4)nc(-c4ccc5ccc6ccccc6c5c4)n3)cc2)cc1 ; c1ccc2cc(-c3nc(-c4ccc5c(ccc6ccccc65)c4)nc(-c4cccc5oc6c(-c7cccc8ccccc78)cccc6c45)n3)ccc2c1]

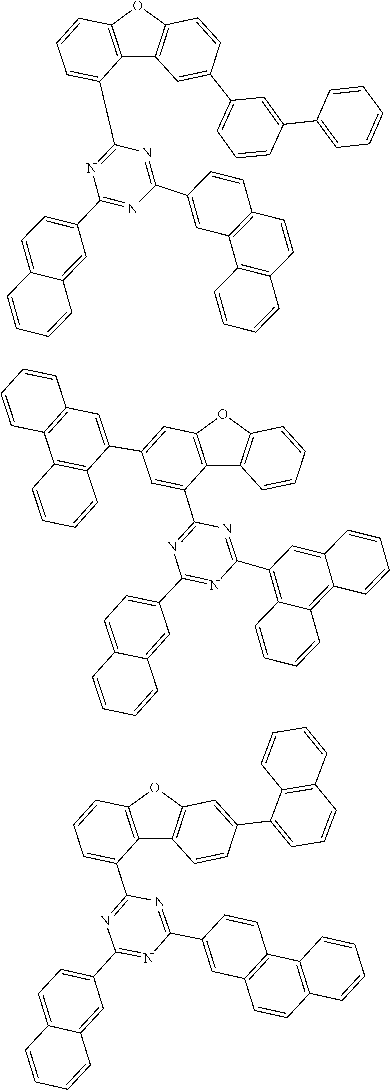

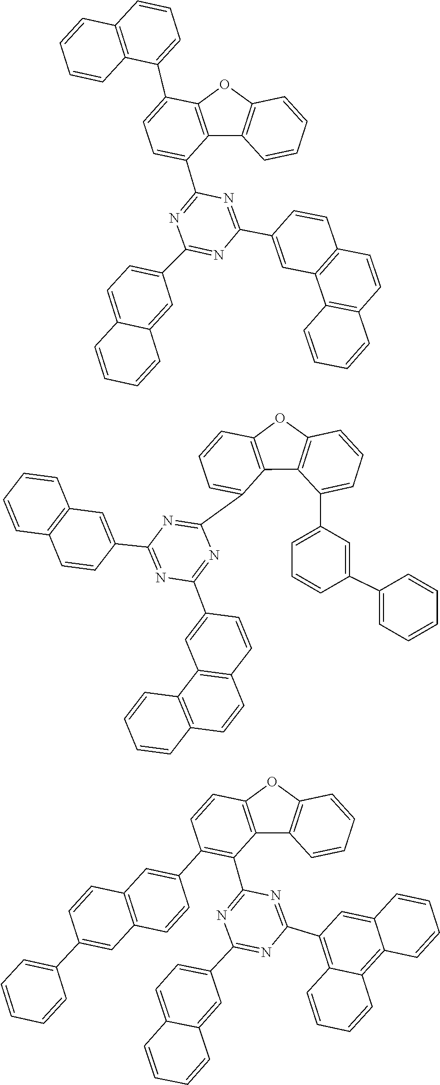 [SMILES: c1ccc2cc(-c3nc(-c4ccc5ccc6ccccc6c5c4)nc(-c4ccc(-c5cccc6ccccc56)c5oc6ccccc6c45)n3)ccc2c1 ; c1ccc(-c2cccc(-c3cccc4oc5cccc(-c6nc(-c7ccc8ccccc8c7)nc(-c7ccc8ccc9ccccc9c8c7)n6)c5c34)c2)cc1 ; c1ccc(-c2ccc3cc(-c4ccc5oc6ccccc6c5c4-c4nc(-c5ccc6ccccc6c5)nc(-c5cc6ccccc6c6ccccc56)n4)ccc3c2)cc1]

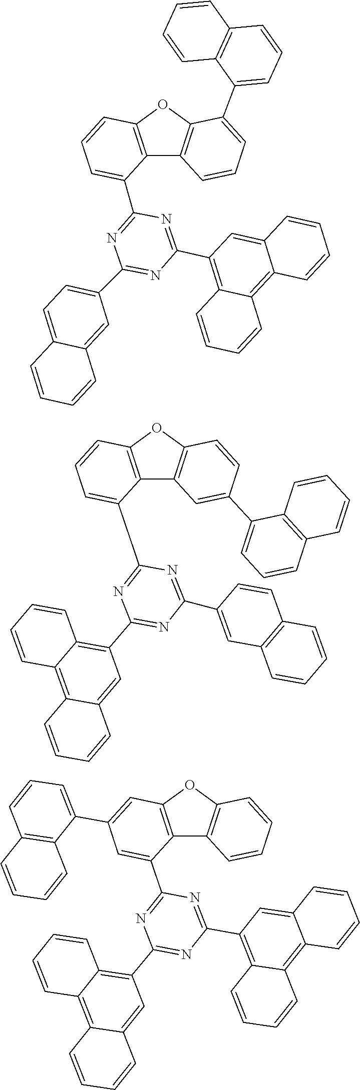

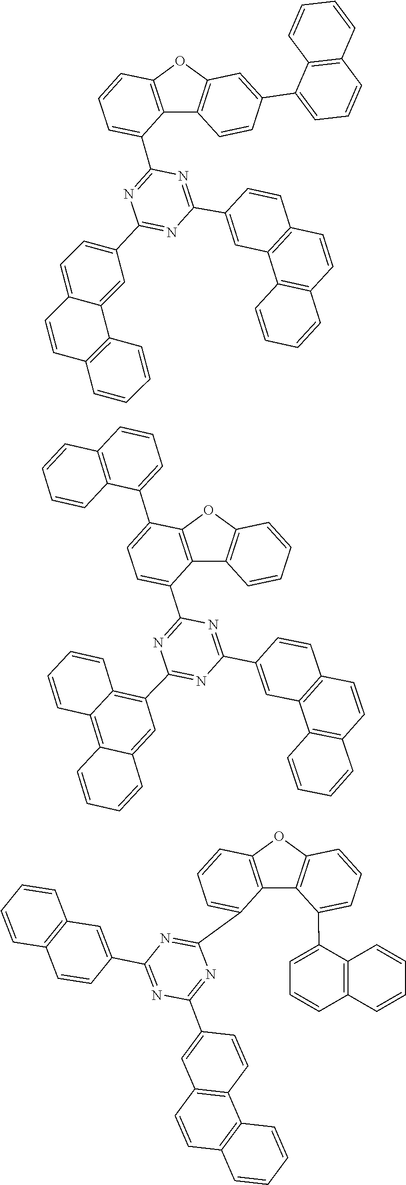

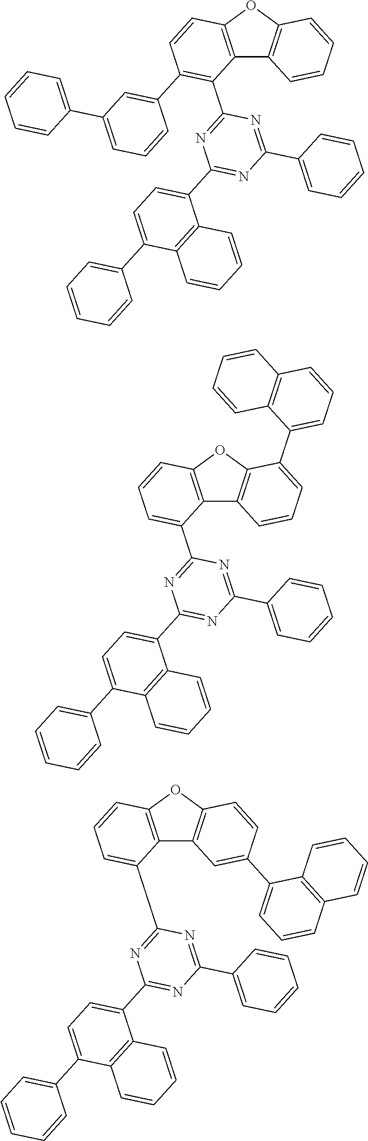 [SMILES: c1ccc(-c2cccc(-c3ccc4oc5ccccc5c4c3-c3nc(-c4ccccc4)nc(-c4ccc(-c5ccccc5)c5ccccc45)n3)c2)cc1 ; c1ccc(-c2nc(-c3ccc(-c4ccccc4)c4ccccc34)nc(-c3cccc4oc5c(-c6cccc7ccccc67)cccc5c34)n2)cc1 ; c1ccc(-c2nc(-c3ccc(-c4ccccc4)c4ccccc34)nc(-c3cccc4oc5ccc(-c6cccc7ccccc67)cc5c34)n2)cc1]

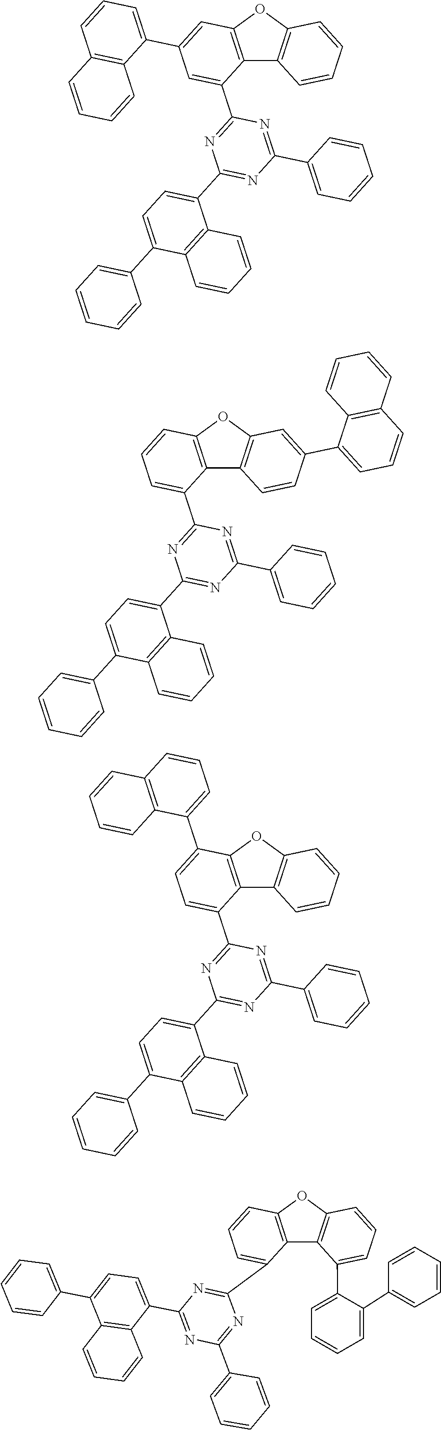

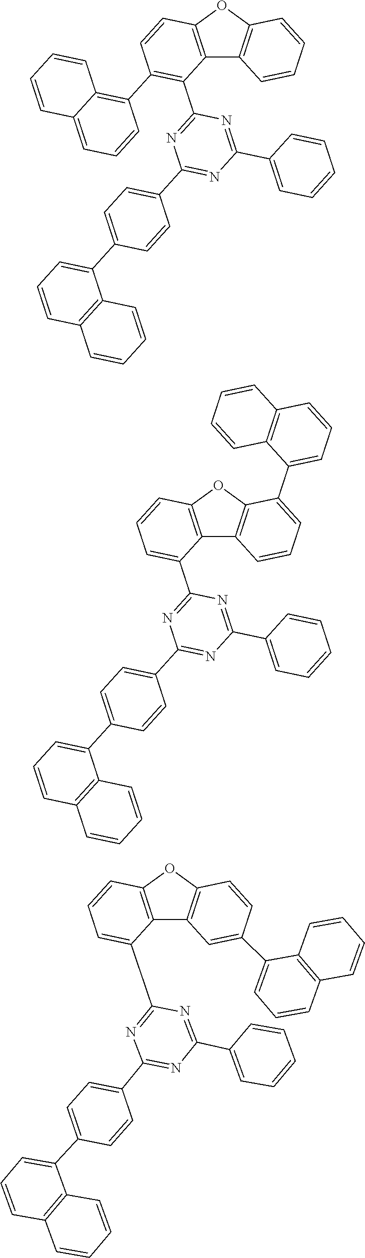

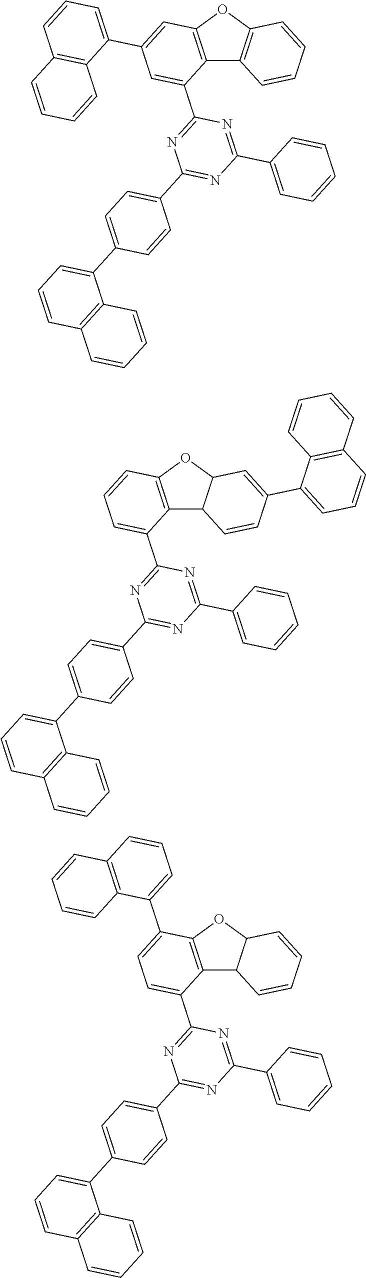

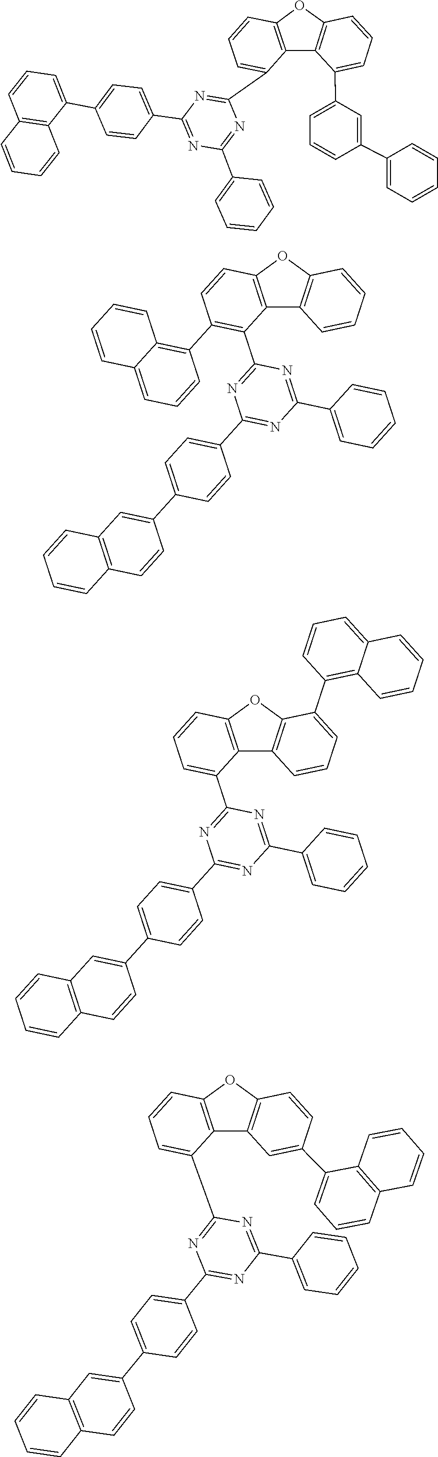

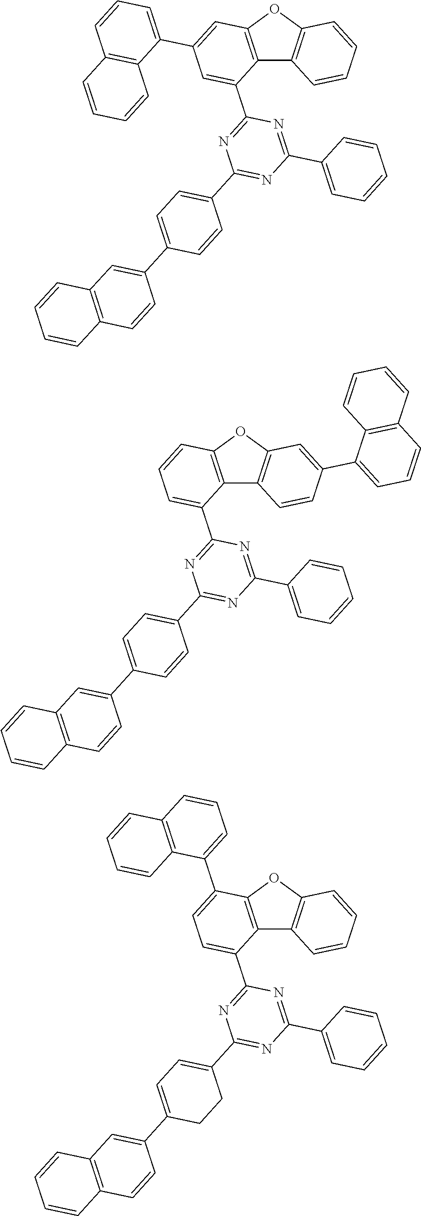 [SMILES: c1ccc(-c2nc(-c3ccc(-c4ccc5ccccc5c4)cc3)nc(-c3cc(-c4cccc5ccccc45)cc4oc5ccccc5c34)n2)cc1 ; c1ccc(-c2nc(-c3ccc(-c4ccc5ccccc5c4)cc3)nc(-c3cccc4oc5cc(-c6cccc7ccccc67)ccc5c34)n2)cc1 ; C1=C(c2ccc3ccccc3c2)CCC(c2nc(-c3ccccc3)nc(-c3ccc(-c4cccc5ccccc45)c4oc5ccccc5c34)n2)=C1]

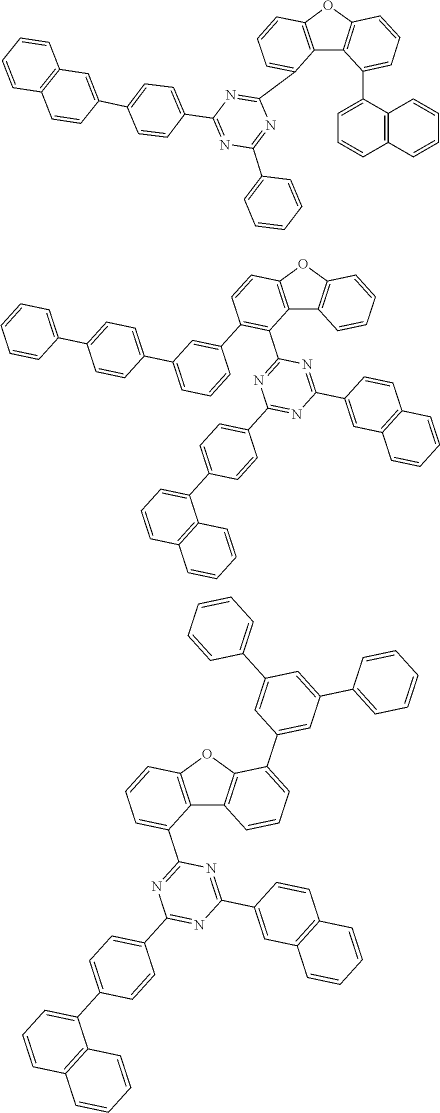

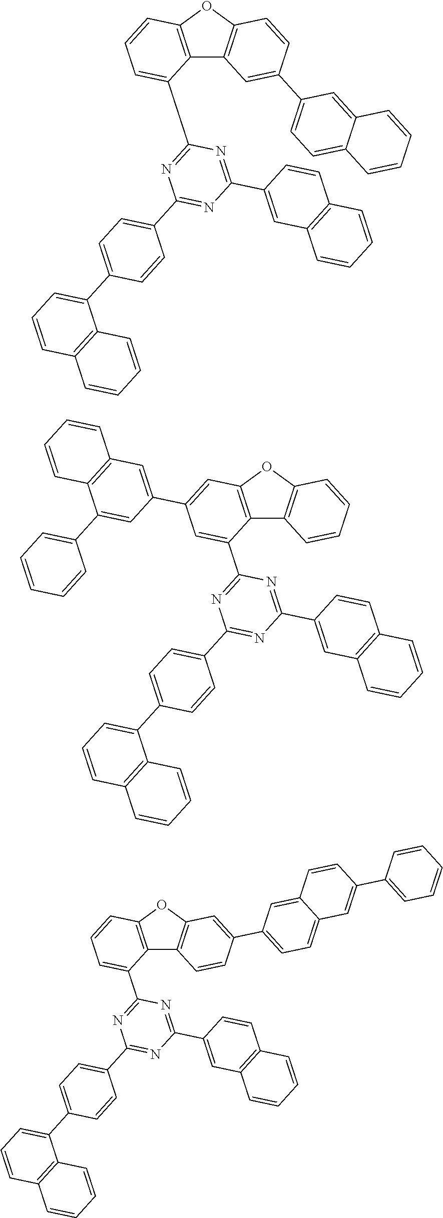

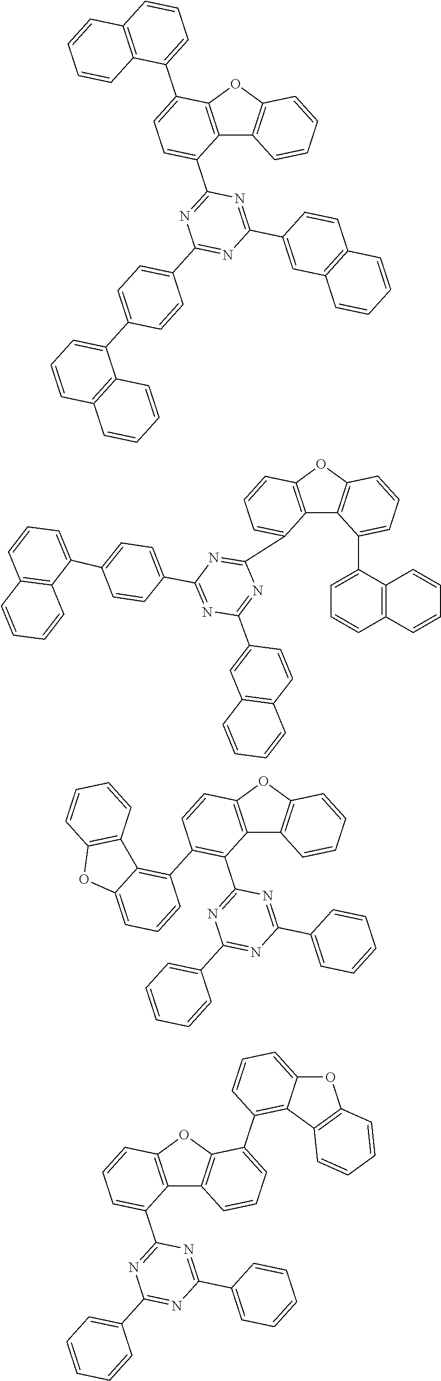

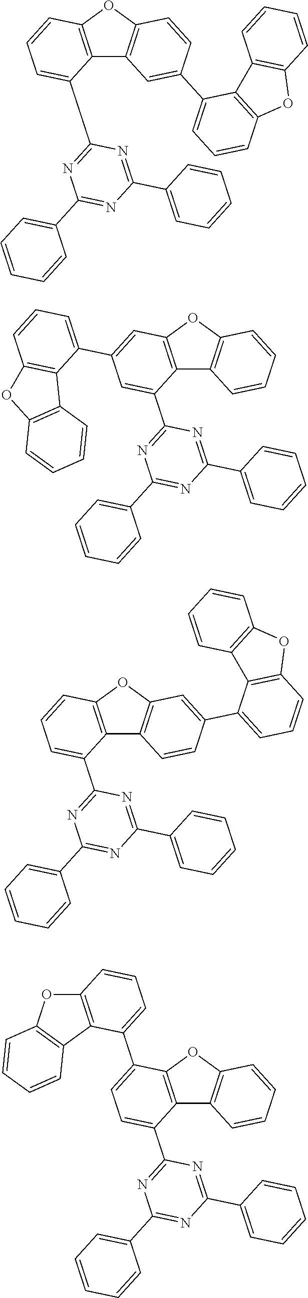

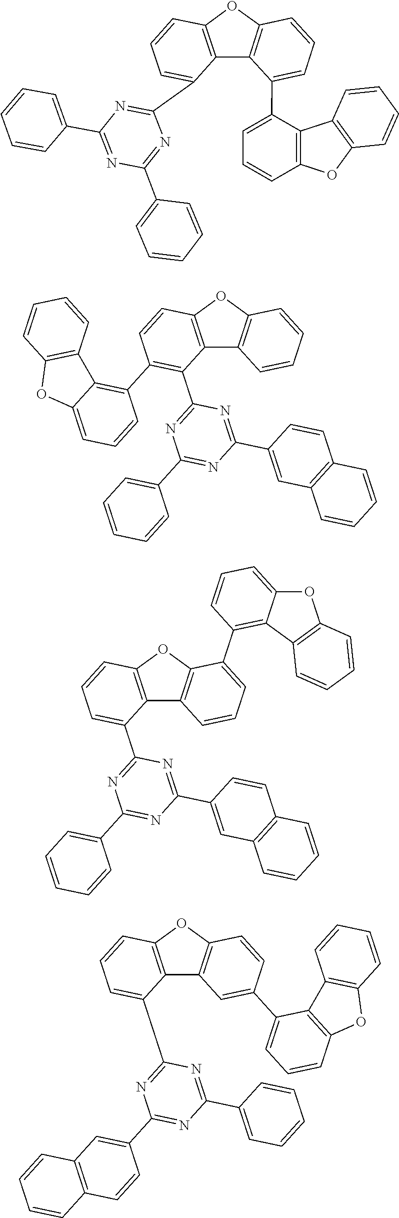 [SMILES: c1ccc(-c2nc(-c3ccccc3)nc(-c3cccc4oc5cccc(-c6cccc7oc8ccccc8c67)c5c34)n2)cc1 ; c1ccc(-c2nc(-c3ccc4ccccc4c3)nc(-c3c(-c4cccc5oc6ccccc6c45)ccc4oc5ccccc5c34)n2)cc1 ; c1ccc(-c2nc(-c3ccc4ccccc4c3)nc(-c3cccc4oc5c(-c6cccc7oc8ccccc8c67)cccc5c34)n2)cc1 ; c1ccc(-c2nc(-c3ccc4ccccc4c3)nc(-c3cccc4oc5ccc(-c6cccc7oc8ccccc8c67)cc5c34)n2)cc1]

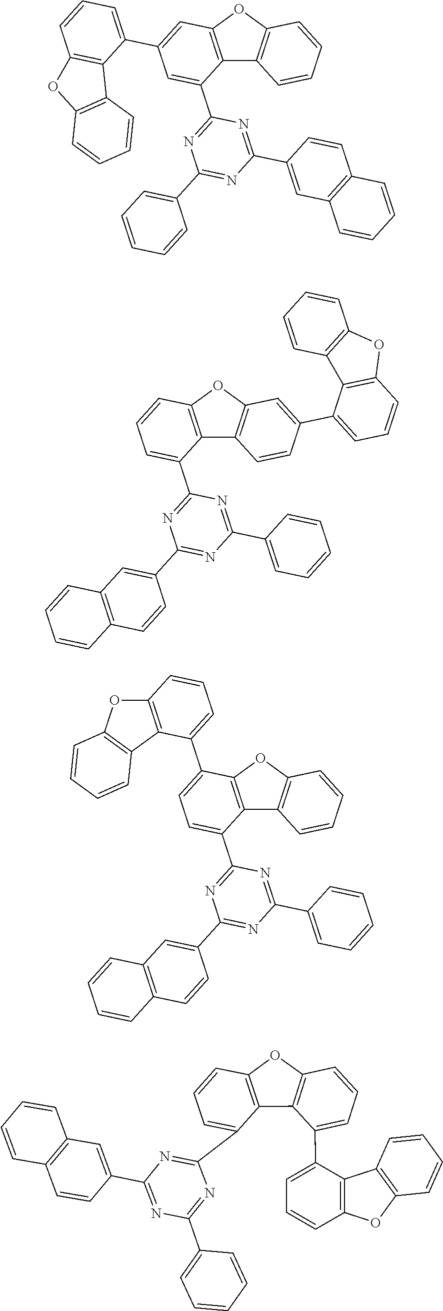 [SMILES: c1ccc(-c2nc(-c3ccc4ccccc4c3)nc(-c3cc(-c4cccc5oc6ccccc6c45)cc4oc5ccccc5c34)n2)cc1 ; c1ccc(-c2nc(-c3ccc4ccccc4c3)nc(-c3cccc4oc5cc(-c6cccc7oc8ccccc8c67)ccc5c34)n2)cc1 ; c1ccc(-c2nc(-c3ccc4ccccc4c3)nc(-c3ccc(-c4cccc5oc6ccccc6c45)c4oc5ccccc5c34)n2)cc1 ; c1ccc(-c2nc(-c3ccc4ccccc4c3)nc(-c3cccc4oc5cccc(-c6cccc7oc8ccccc8c67)c5c34)n2)cc1]

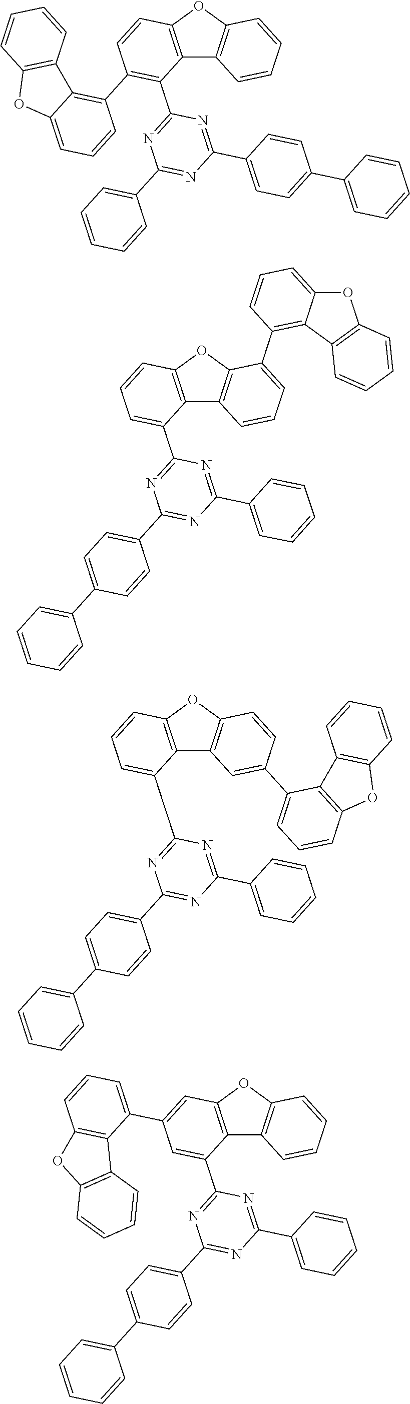

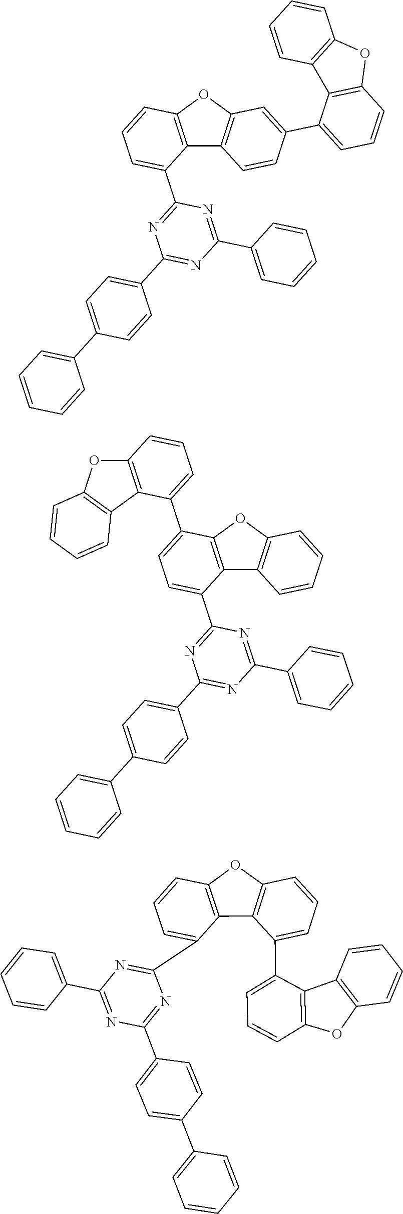 [SMILES: c1ccc(-c2ccc(-c3nc(-c4ccccc4)nc(-c4cccc5oc6cc(-c7cccc8oc9ccccc9c78)ccc6c45)n3)cc2)cc1 ; c1ccc(-c2ccc(-c3nc(-c4ccccc4)nc(-c4ccc(-c5cccc6oc7ccccc7c56)c5oc6ccccc6c45)n3)cc2)cc1 ; c1ccc(-c2ccc(-c3nc(-c4ccccc4)nc(-c4cccc5oc6cccc(-c7cccc8oc9ccccc9c78)c6c45)n3)cc2)cc1]

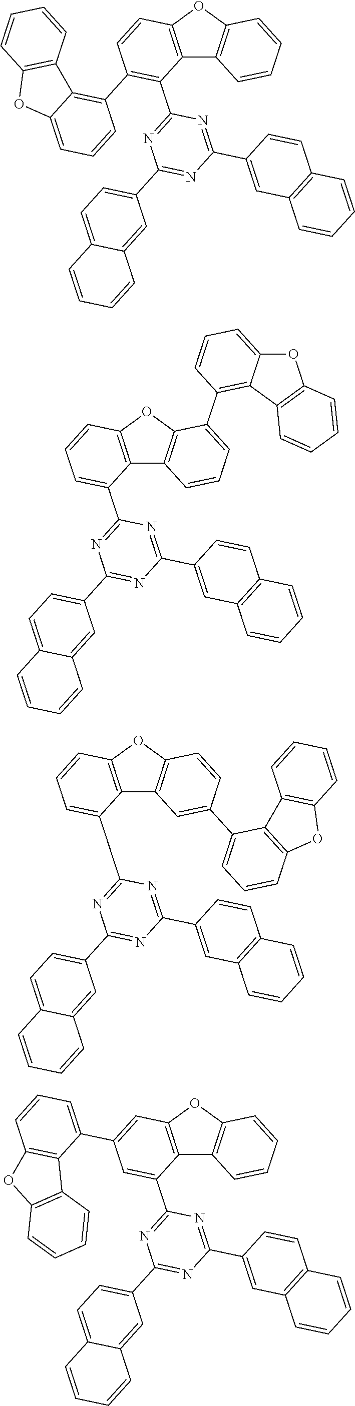

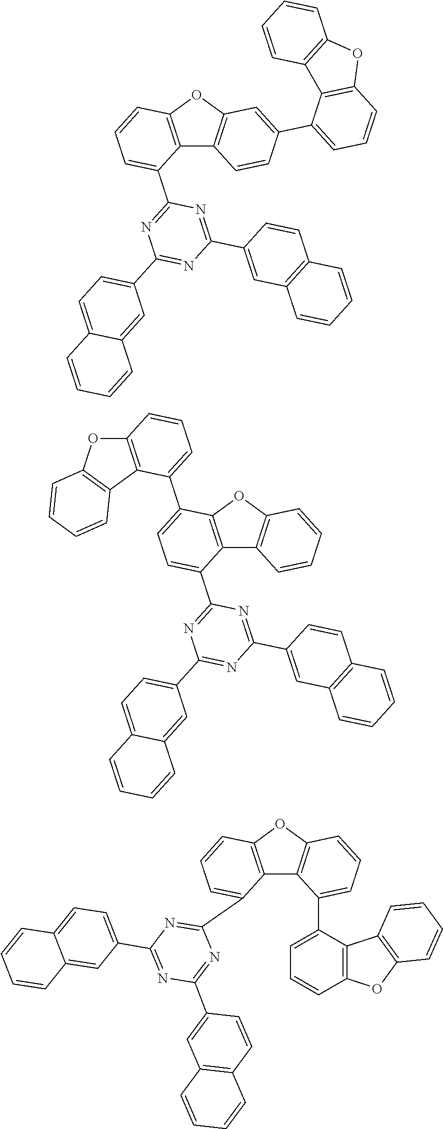

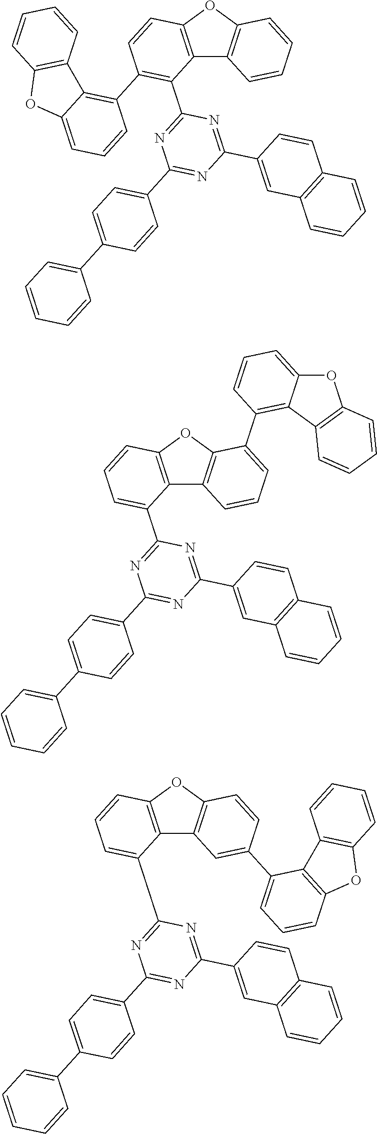 [SMILES: c1ccc(-c2ccc(-c3nc(-c4ccc5ccccc5c4)nc(-c4c(-c5cccc6oc7ccccc7c56)ccc5oc6ccccc6c45)n3)cc2)cc1 ; c1ccc(-c2ccc(-c3nc(-c4ccc5ccccc5c4)nc(-c4cccc5oc6c(-c7cccc8oc9ccccc9c78)cccc6c45)n3)cc2)cc1 ; c1ccc(-c2ccc(-c3nc(-c4ccc5ccccc5c4)nc(-c4cccc5oc6ccc(-c7cccc8oc9ccccc9c78)cc6c45)n3)cc2)cc1]

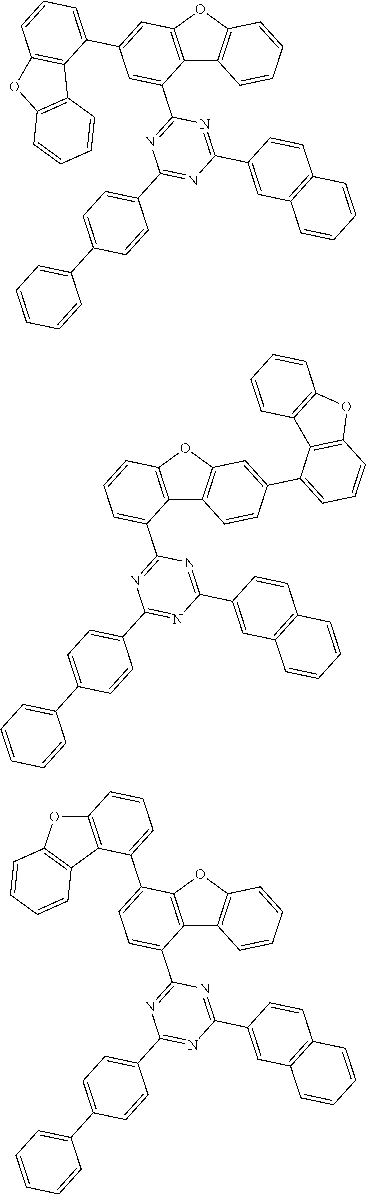 [SMILES: c1ccc(-c2ccc(-c3nc(-c4ccc5ccccc5c4)nc(-c4cc(-c5cccc6oc7ccccc7c56)cc5oc6ccccc6c45)n3)cc2)cc1 ; c1ccc(-c2ccc(-c3nc(-c4ccc5ccccc5c4)nc(-c4cccc5oc6cc(-c7cccc8oc9ccccc9c78)ccc6c45)n3)cc2)cc1 ; c1ccc(-c2ccc(-c3nc(-c4ccc5ccccc5c4)nc(-c4ccc(-c5cccc6oc7ccccc7c56)c5oc6ccccc6c45)n3)cc2)cc1]

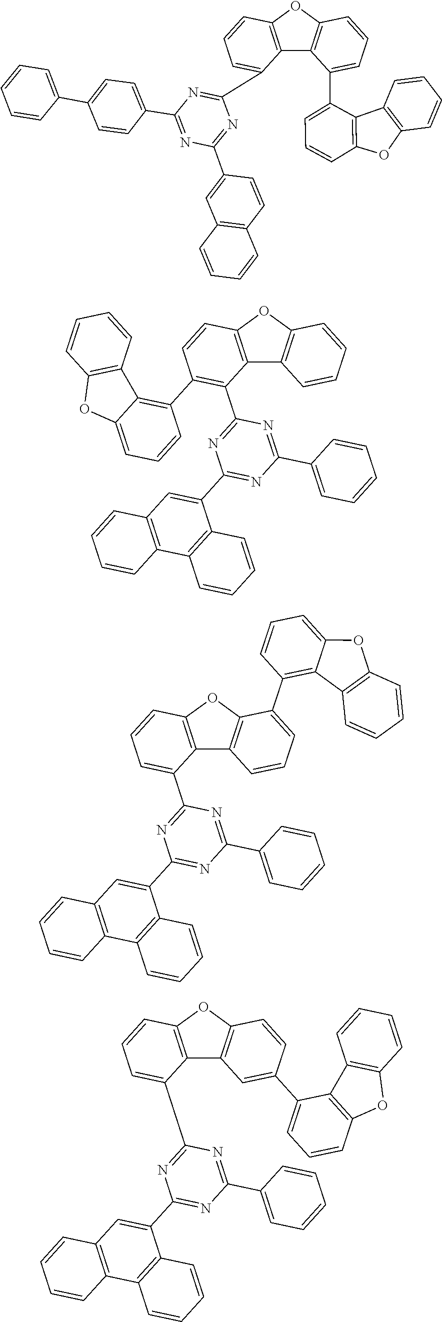

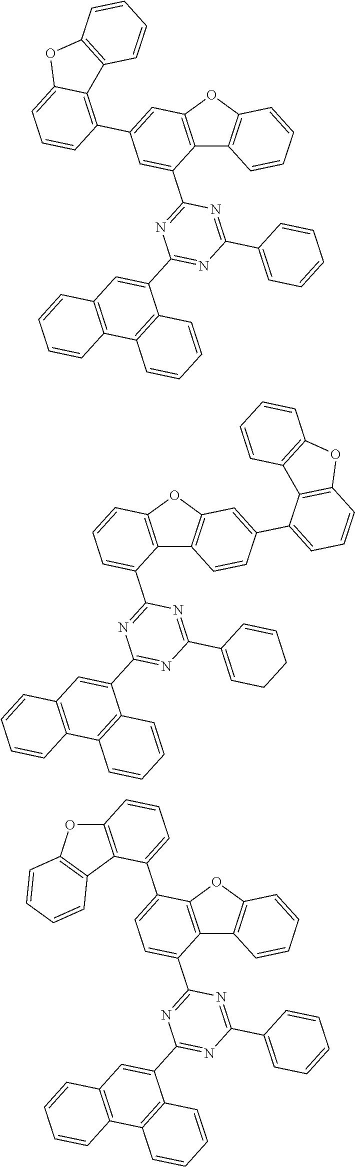

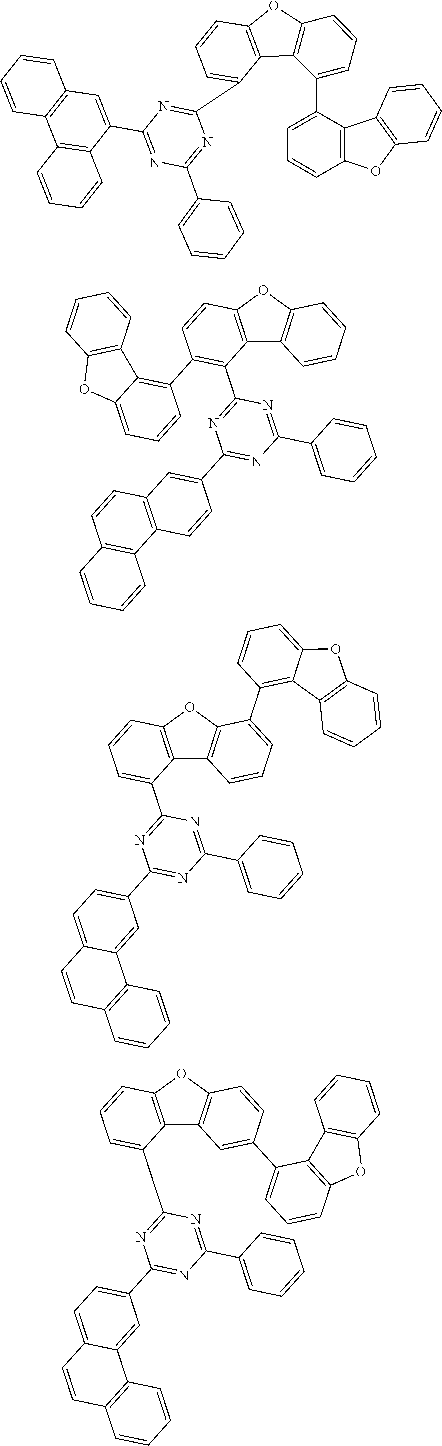

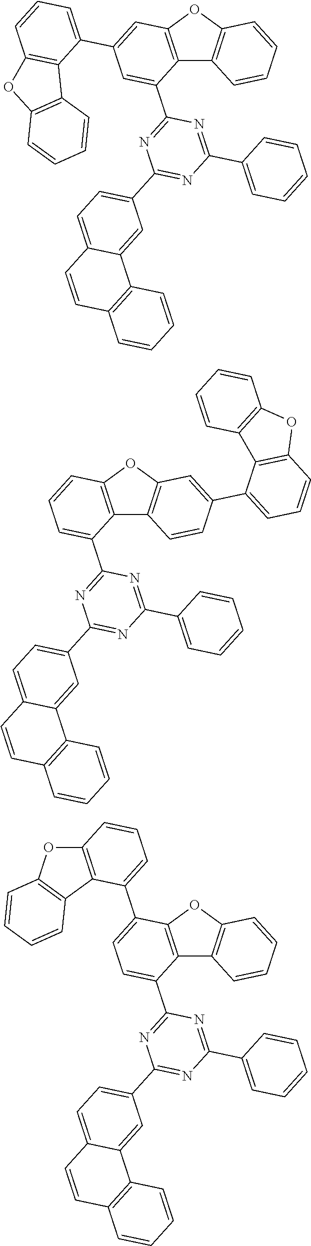

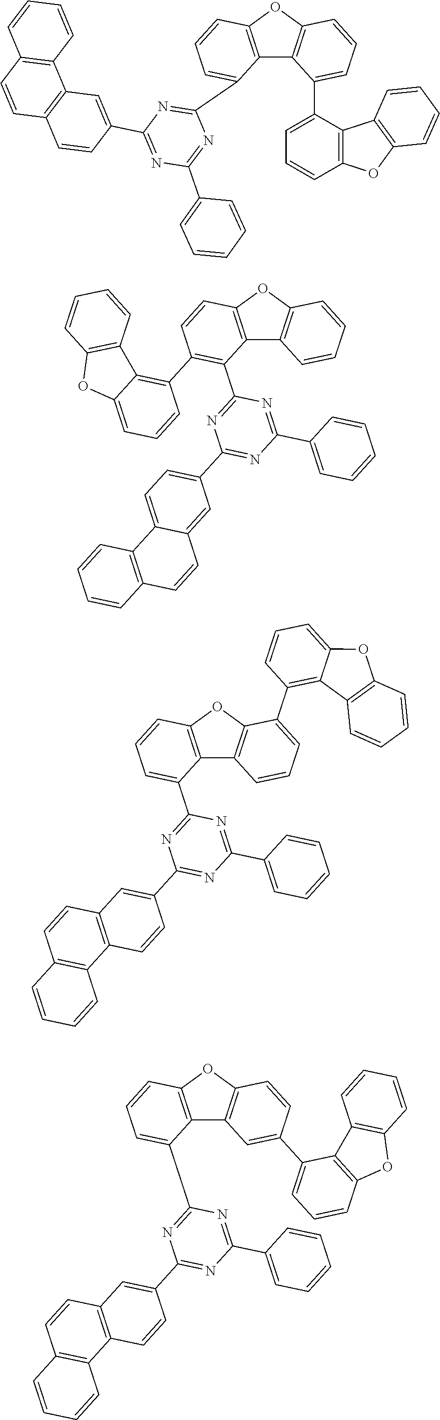 [SMILES: c1ccc(-c2nc(-c3ccc4ccc5ccccc5c4c3)nc(-c3cccc4oc5cccc(-c6cccc7oc8ccccc8c67)c5c34)n2)cc1 ; c1ccc(-c2nc(-c3ccc4c(ccc5ccccc54)c3)nc(-c3c(-c4cccc5oc6ccccc6c45)ccc4oc5ccccc5c34)n2)cc1 ; c1ccc(-c2nc(-c3ccc4c(ccc5ccccc54)c3)nc(-c3cccc4oc5c(-c6cccc7oc8ccccc8c67)cccc5c34)n2)cc1 ; c1ccc(-c2nc(-c3ccc4c(ccc5ccccc54)c3)nc(-c3cccc4oc5ccc(-c6cccc7oc8ccccc8c67)cc5c34)n2)cc1]

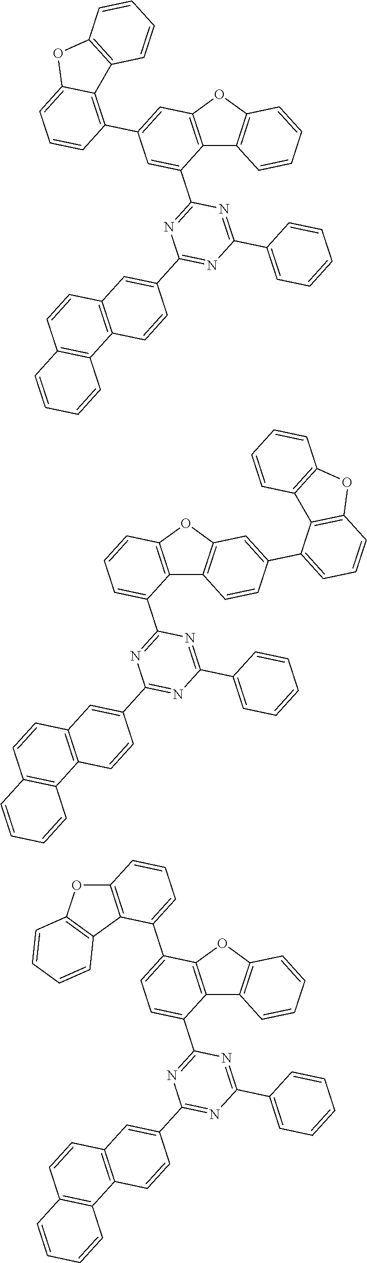 [SMILES: c1ccc(-c2nc(-c3ccc4c(ccc5ccccc54)c3)nc(-c3cc(-c4cccc5oc6ccccc6c45)cc4oc5ccccc5c34)n2)cc1 ; c1ccc(-c2nc(-c3ccc4c(ccc5ccccc54)c3)nc(-c3cccc4oc5cc(-c6cccc7oc8ccccc8c67)ccc5c34)n2)cc1 ; c1ccc(-c2nc(-c3ccc4c(ccc5ccccc54)c3)nc(-c3ccc(-c4cccc5oc6ccccc6c45)c4oc5ccccc5c34)n2)cc1]

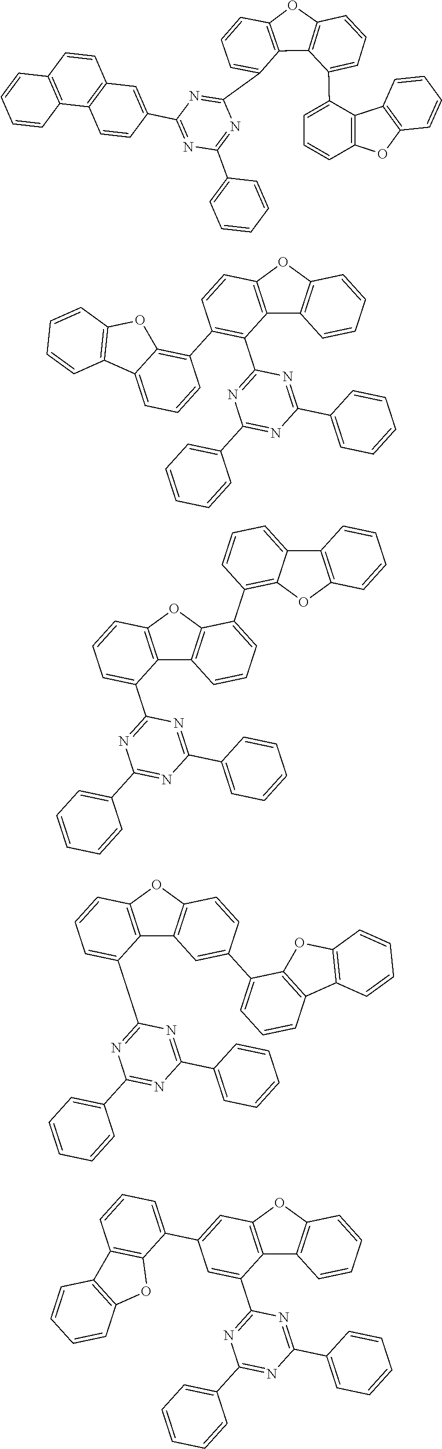

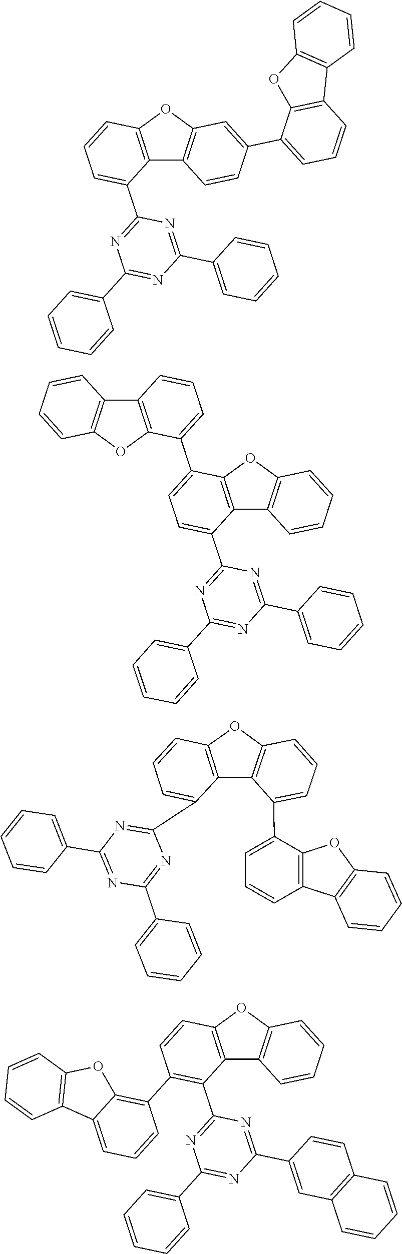

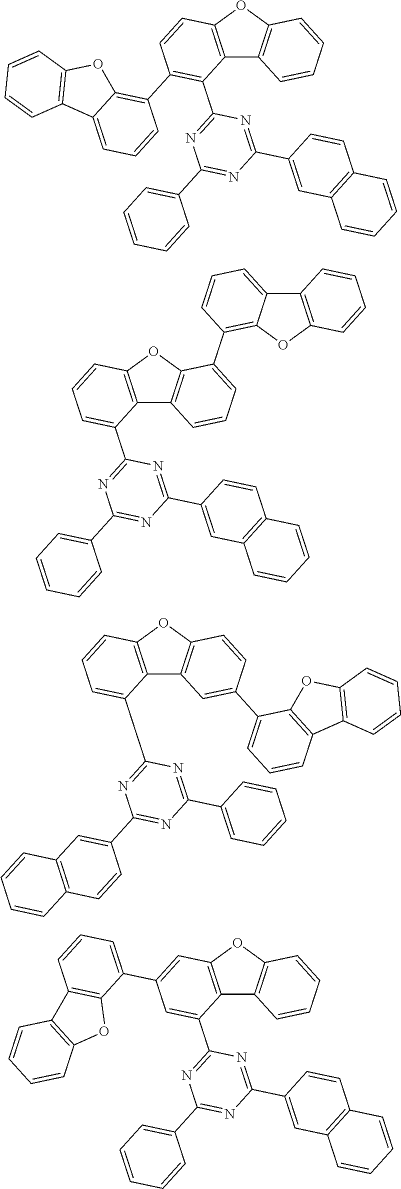

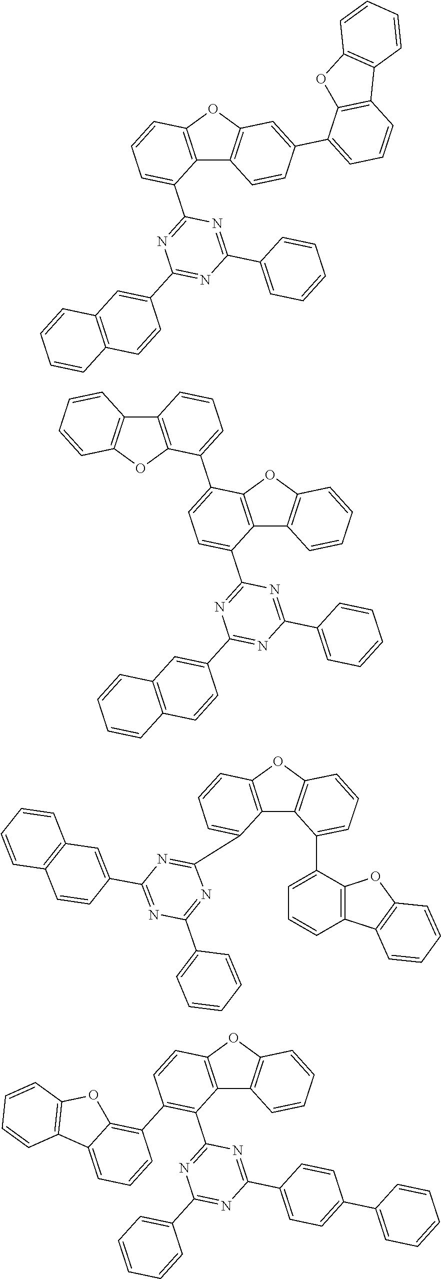

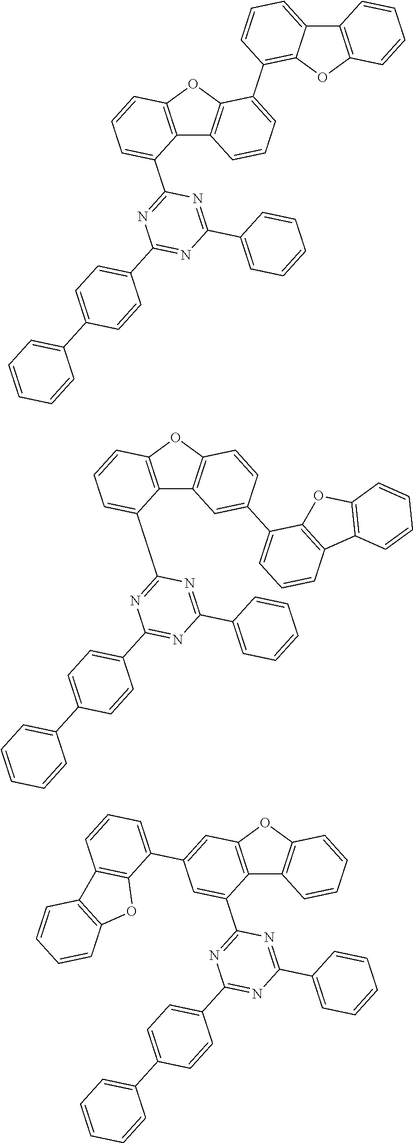 [SMILES: c1ccc(-c2ccc(-c3nc(-c4ccccc4)nc(-c4cccc5oc6c(-c7cccc8c7oc7ccccc78)cccc6c45)n3)cc2)cc1 ; c1ccc(-c2ccc(-c3nc(-c4ccccc4)nc(-c4cccc5oc6ccc(-c7cccc8c7oc7ccccc78)cc6c45)n3)cc2)cc1 ; c1ccc(-c2ccc(-c3nc(-c4ccccc4)nc(-c4cc(-c5cccc6c5oc5ccccc56)cc5oc6ccccc6c45)n3)cc2)cc1]

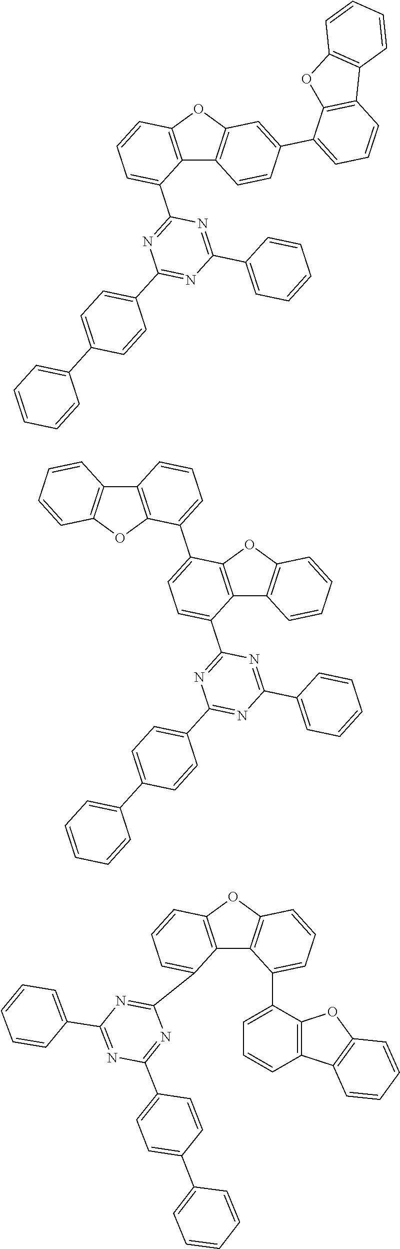

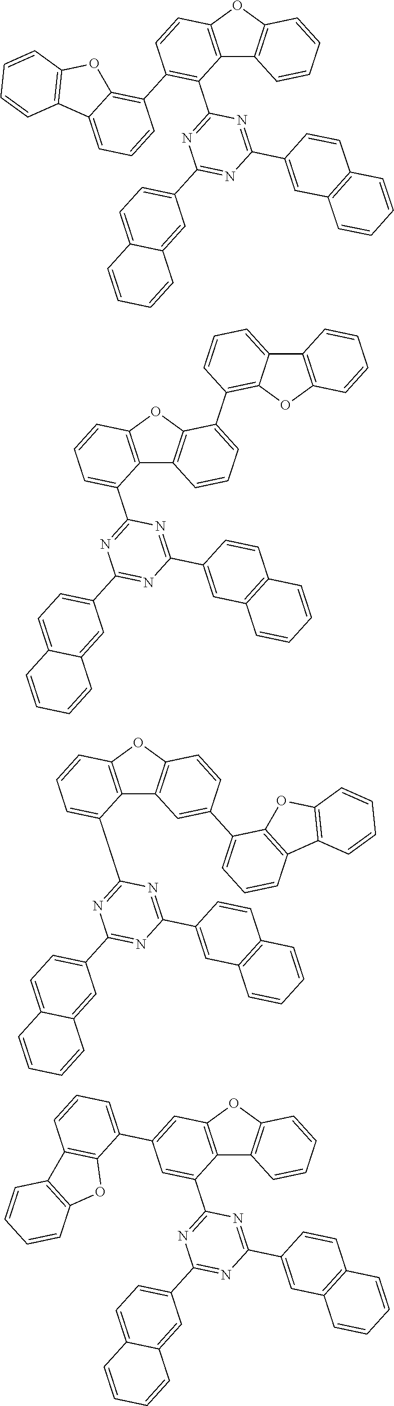

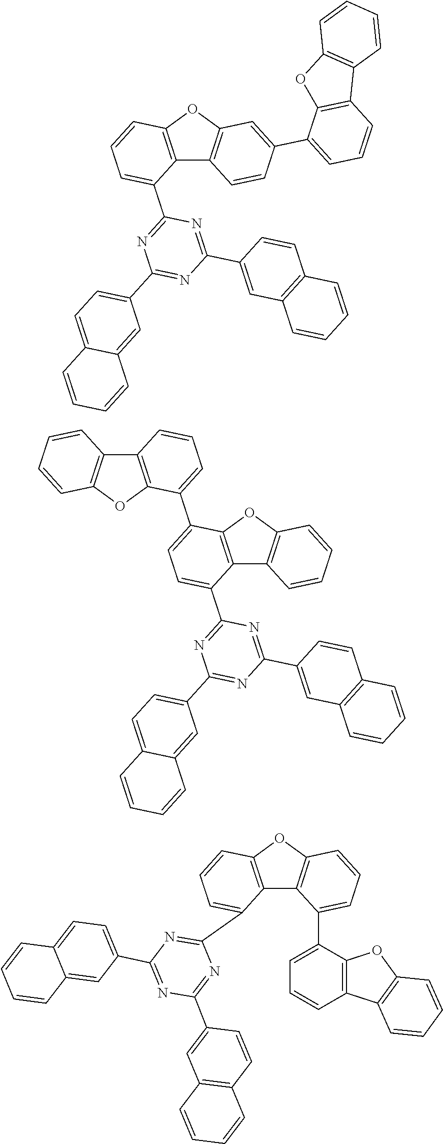

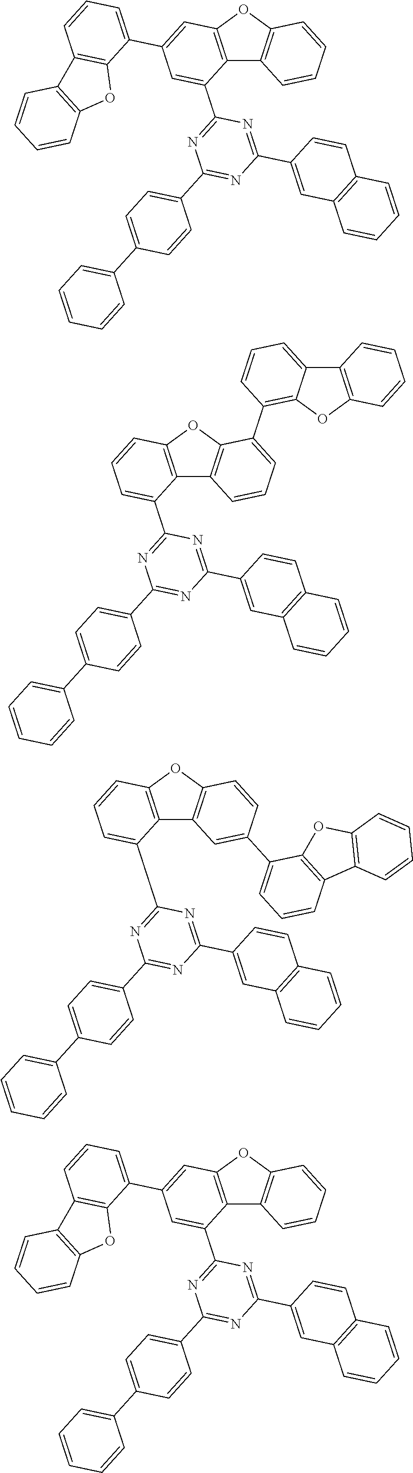 [SMILES: c1ccc(-c2ccc(-c3nc(-c4ccc5ccccc5c4)nc(-c4cc(-c5cccc6c5oc5ccccc56)cc5oc6ccccc6c45)n3)cc2)cc1 ; c1ccc(-c2ccc(-c3nc(-c4ccc5ccccc5c4)nc(-c4cccc5oc6c(-c7cccc8c7oc7ccccc78)cccc6c45)n3)cc2)cc1 ; c1ccc(-c2ccc(-c3nc(-c4ccc5ccccc5c4)nc(-c4cccc5oc6ccc(-c7cccc8c7oc7ccccc78)cc6c45)n3)cc2)cc1 ; c1ccc(-c2ccc(-c3nc(-c4ccc5ccccc5c4)nc(-c4cc(-c5cccc6c5oc5ccccc56)cc5oc6ccccc6c45)n3)cc2)cc1]

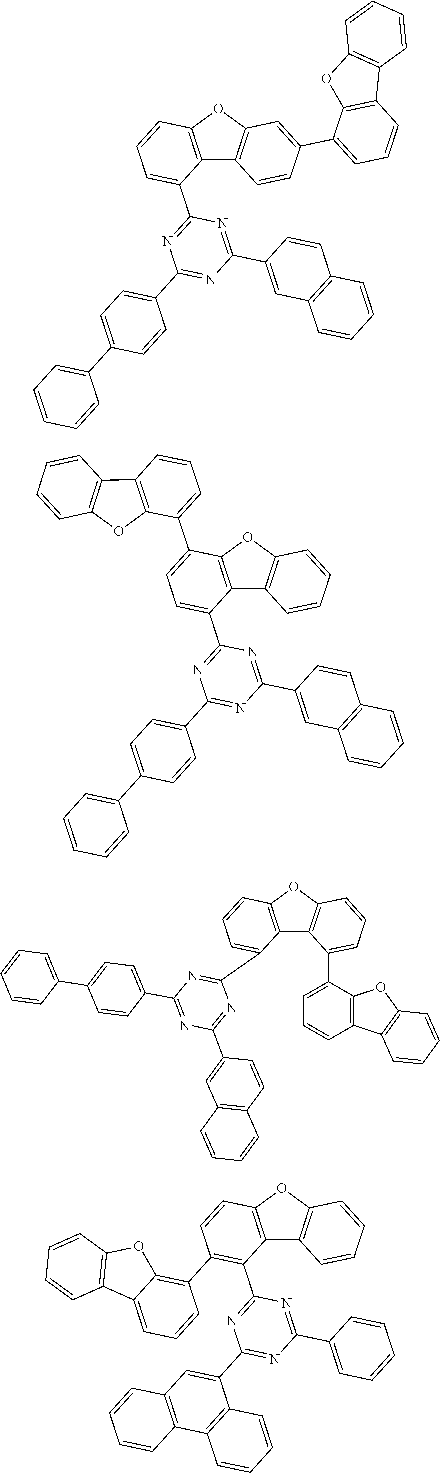

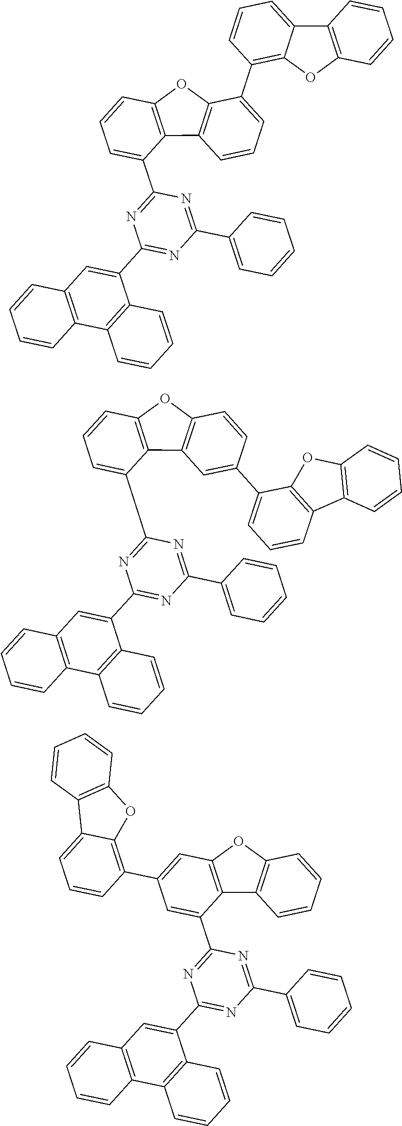

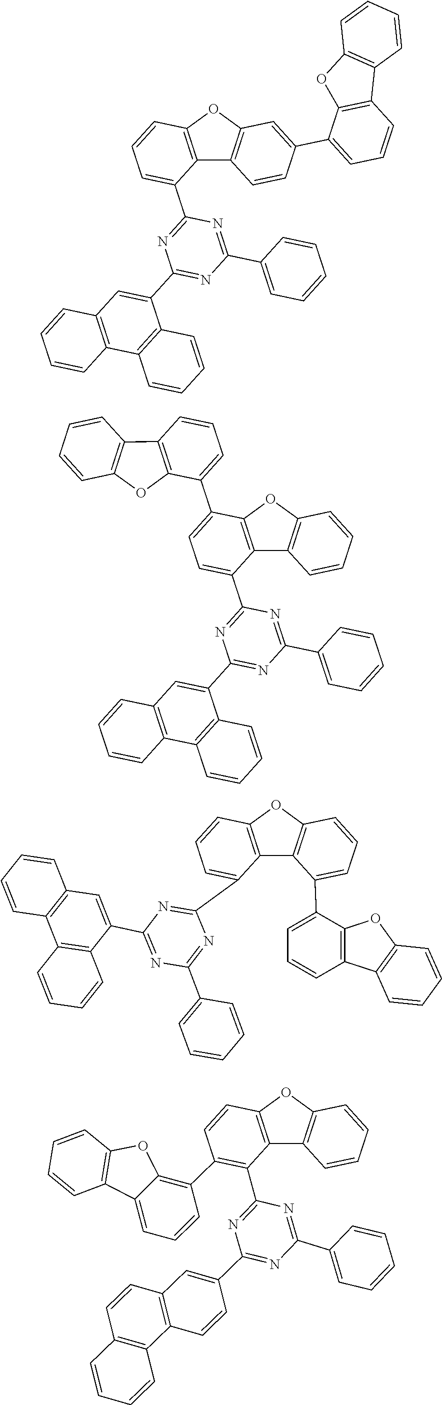

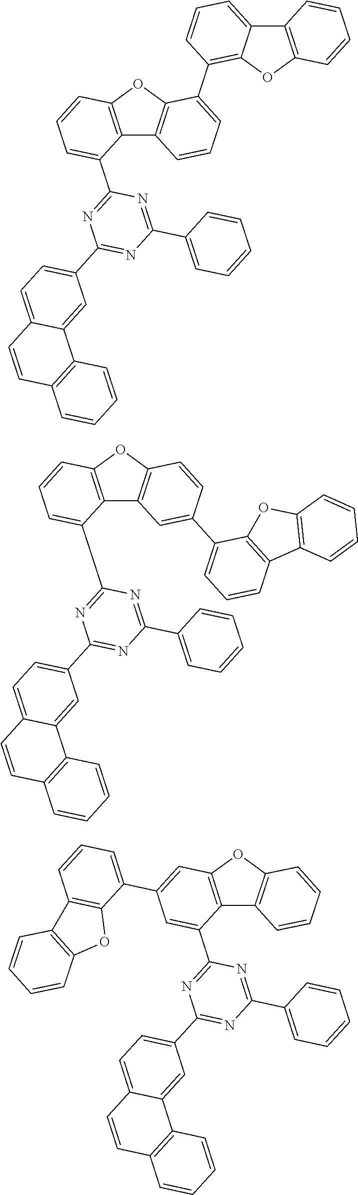

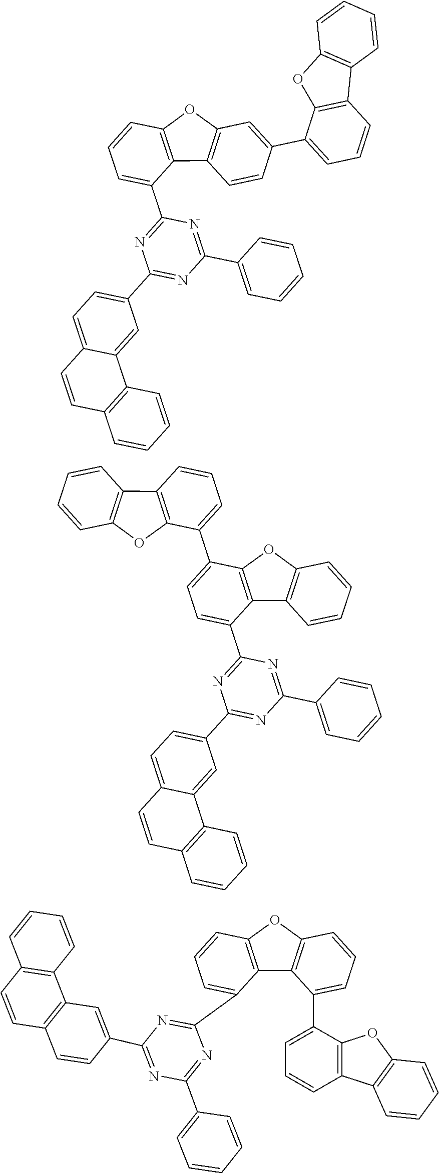

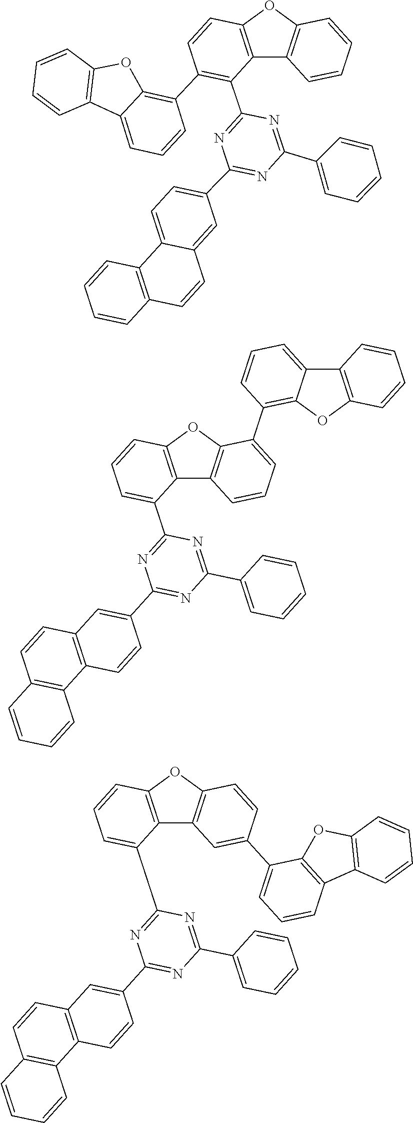 [SMILES: c1ccc(-c2nc(-c3ccc4c(ccc5ccccc54)c3)nc(-c3c(-c4cccc5c4oc4ccccc45)ccc4oc5ccccc5c34)n2)cc1 ; c1ccc(-c2nc(-c3ccc4c(ccc5ccccc54)c3)nc(-c3cccc4oc5c(-c6cccc7c6oc6ccccc67)cccc5c34)n2)cc1 ; c1ccc(-c2nc(-c3ccc4c(ccc5ccccc54)c3)nc(-c3cccc4oc5ccc(-c6cccc7c6oc6ccccc67)cc5c34)n2)cc1]

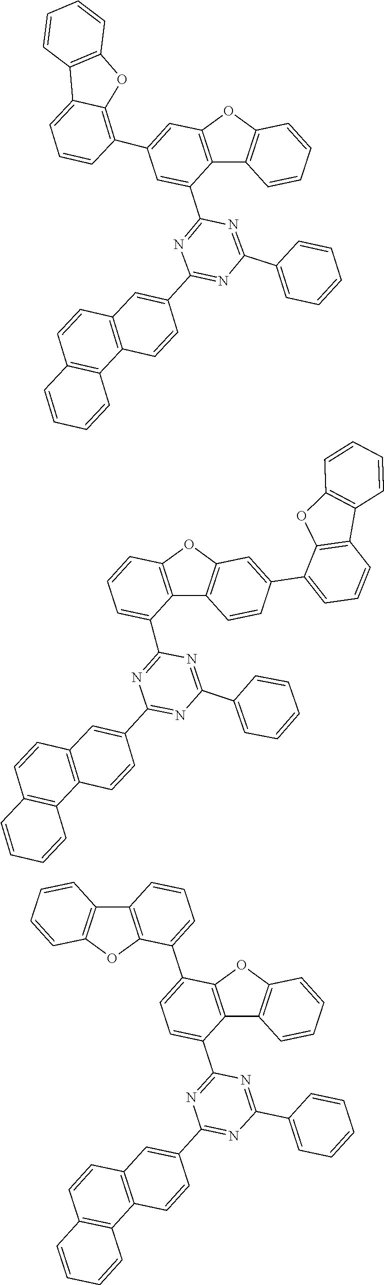

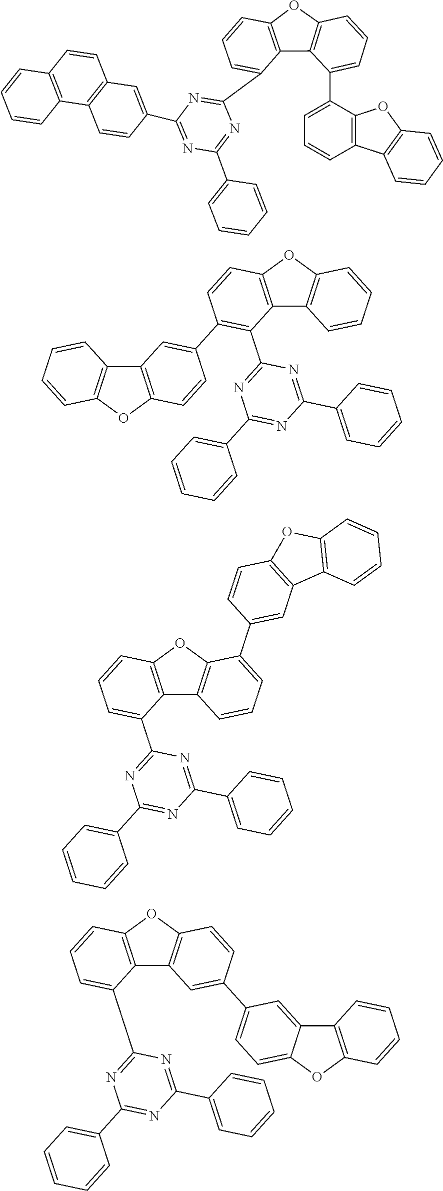

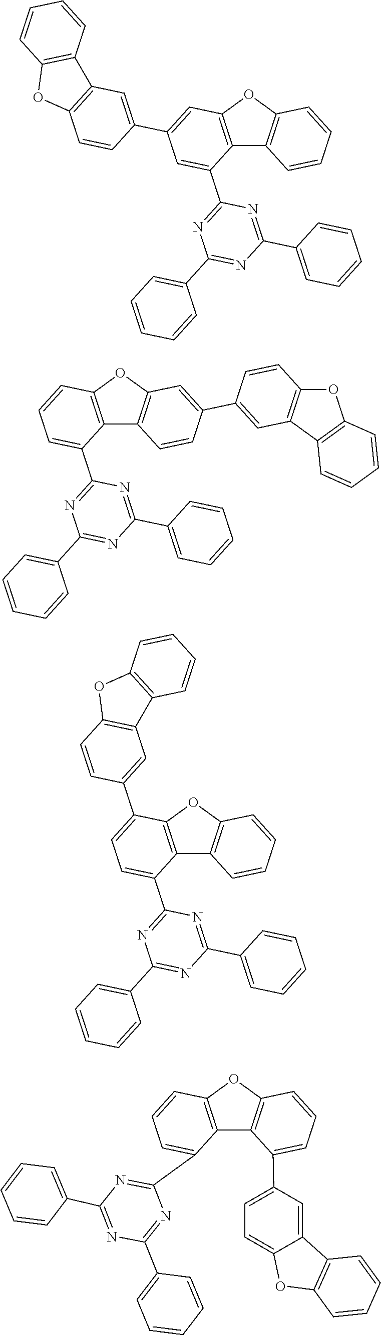

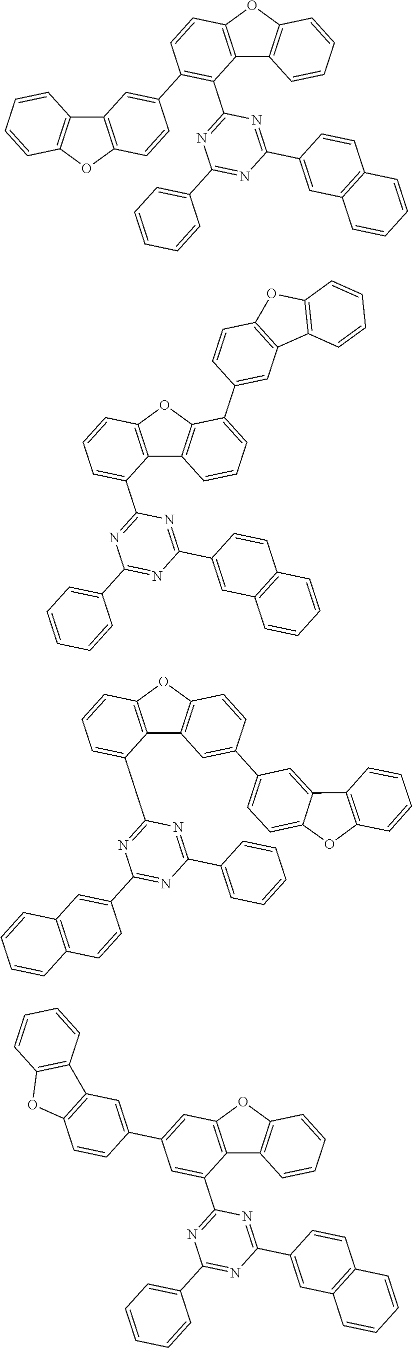

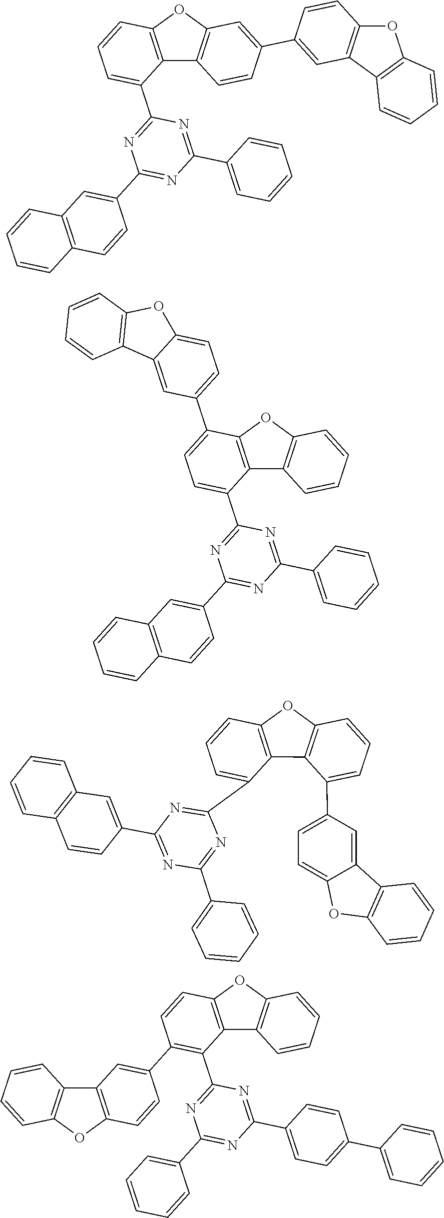

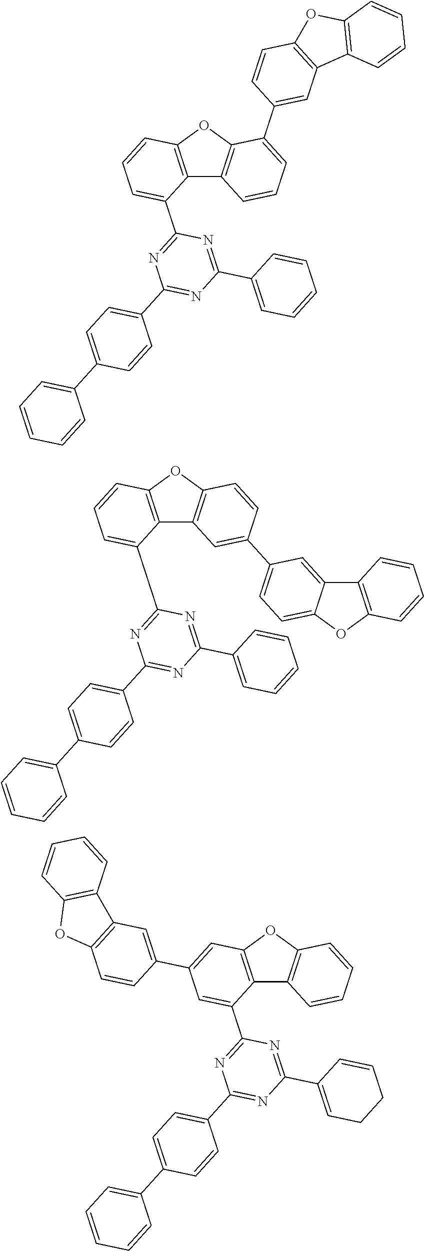

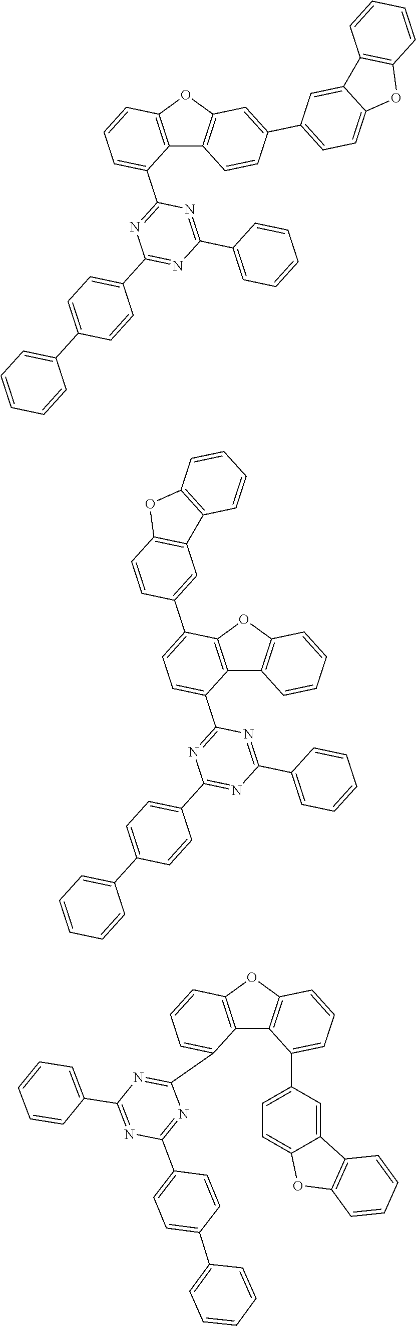 [SMILES: c1ccc(-c2ccc(-c3nc(-c4ccccc4)nc(-c4cccc5oc6cc(-c7ccc8oc9ccccc9c8c7)ccc6c45)n3)cc2)cc1 ; c1ccc(-c2ccc(-c3nc(-c4ccccc4)nc(-c4ccc(-c5ccc6oc7ccccc7c6c5)c5oc6ccccc6c45)n3)cc2)cc1 ; c1ccc(-c2ccc(-c3nc(-c4ccccc4)nc(-c4cccc5oc6cccc(-c7ccc8oc9ccccc9c8c7)c6c45)n3)cc2)cc1]

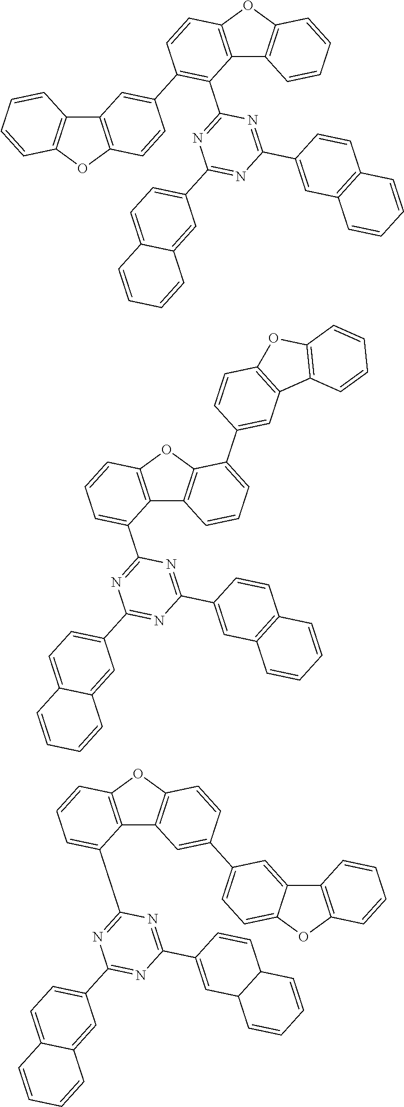

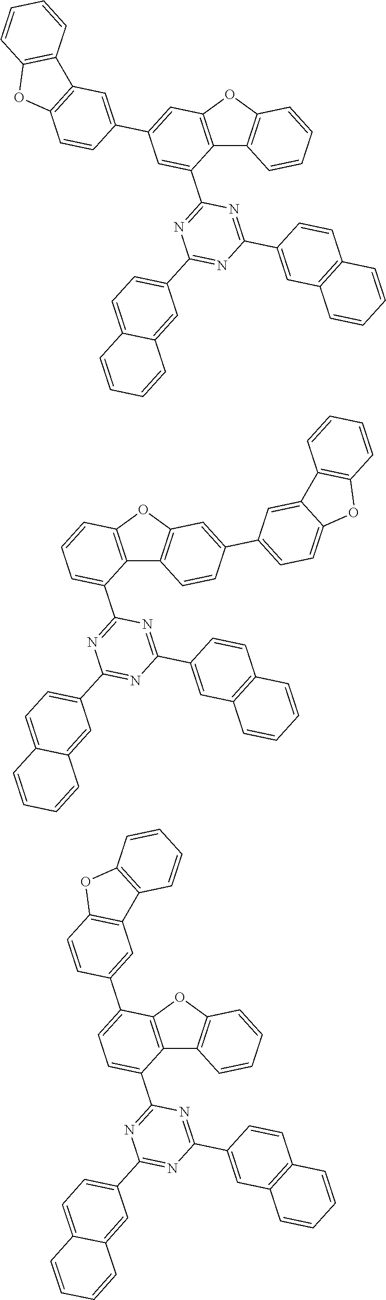 [SMILES: c1ccc2cc(-c3nc(-c4ccc5ccccc5c4)nc(-c4cc(-c5ccc6oc7ccccc7c6c5)cc5oc6ccccc6c45)n3)ccc2c1 ; c1ccc2cc(-c3nc(-c4ccc5ccccc5c4)nc(-c4cccc5oc6cc(-c7ccc8oc9ccccc9c8c7)ccc6c45)n3)ccc2c1 ; c1ccc2cc(-c3nc(-c4ccc5ccccc5c4)nc(-c4ccc(-c5ccc6oc7ccccc7c6c5)c5oc6ccccc6c45)n3)ccc2c1]

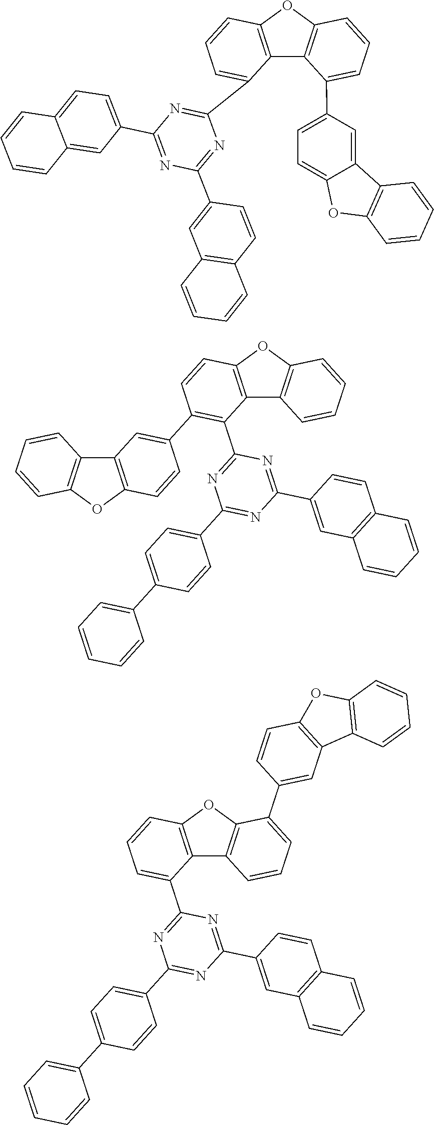

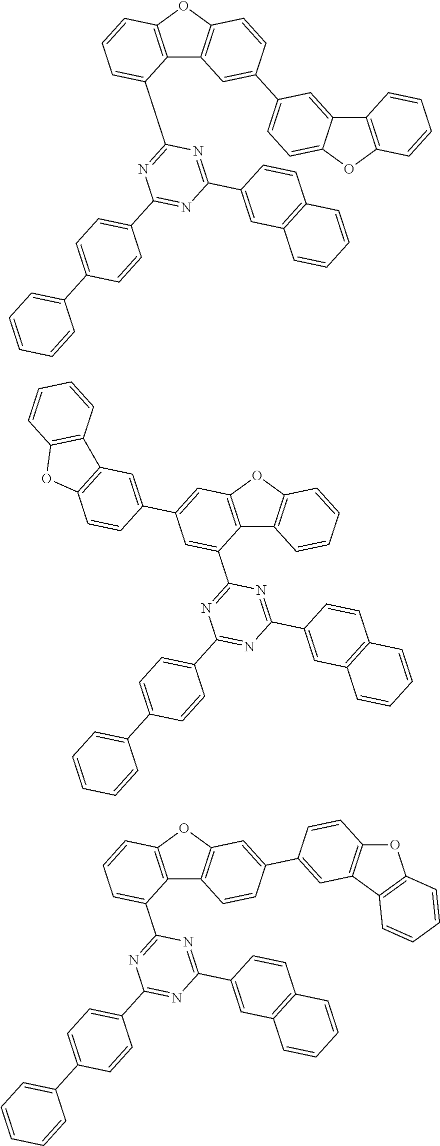

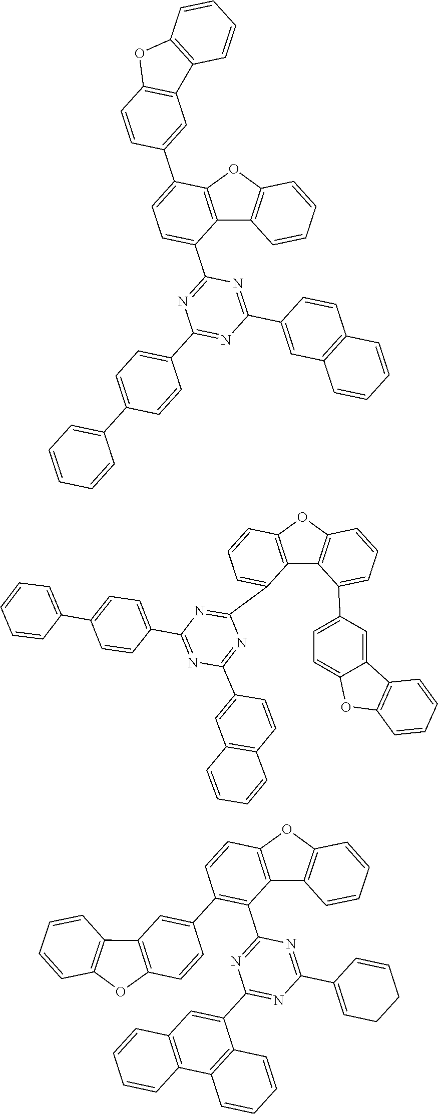

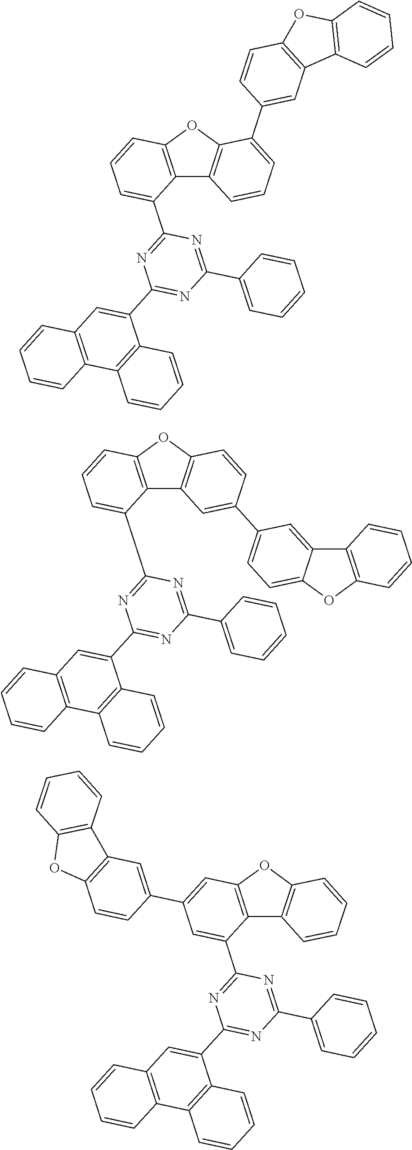

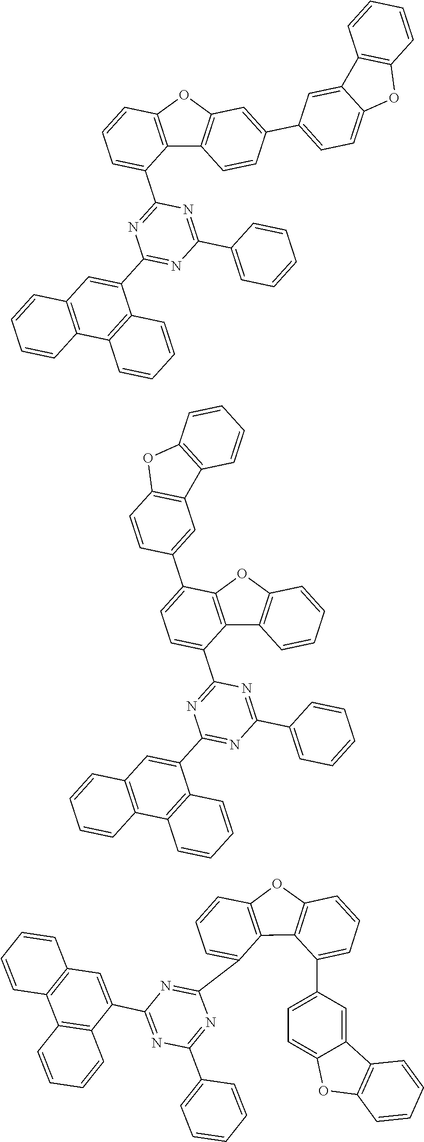

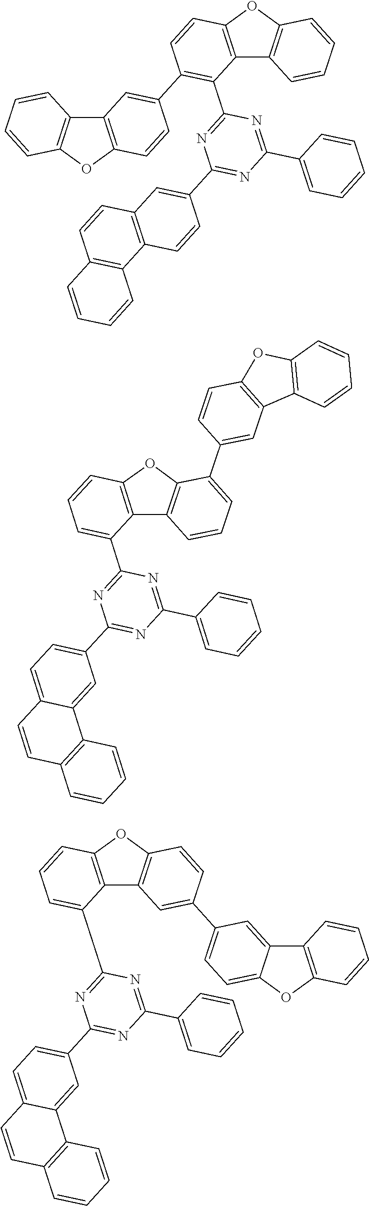

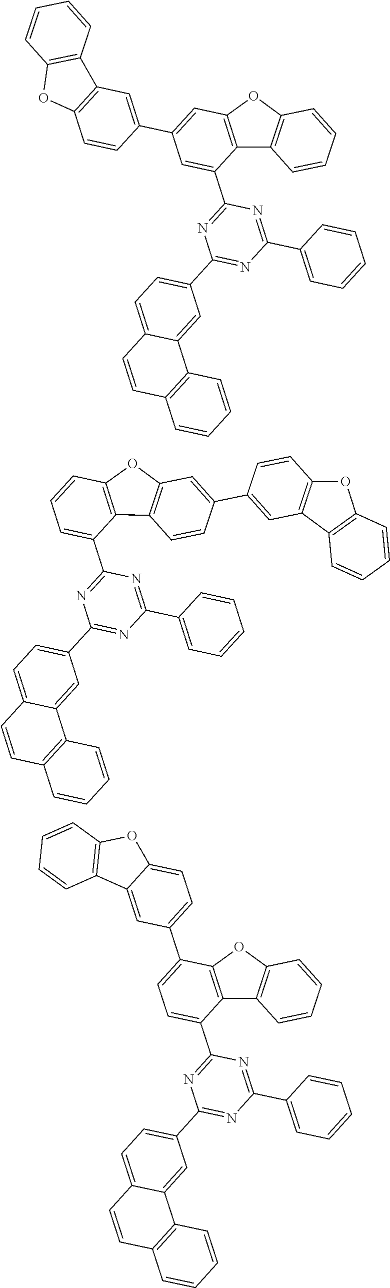

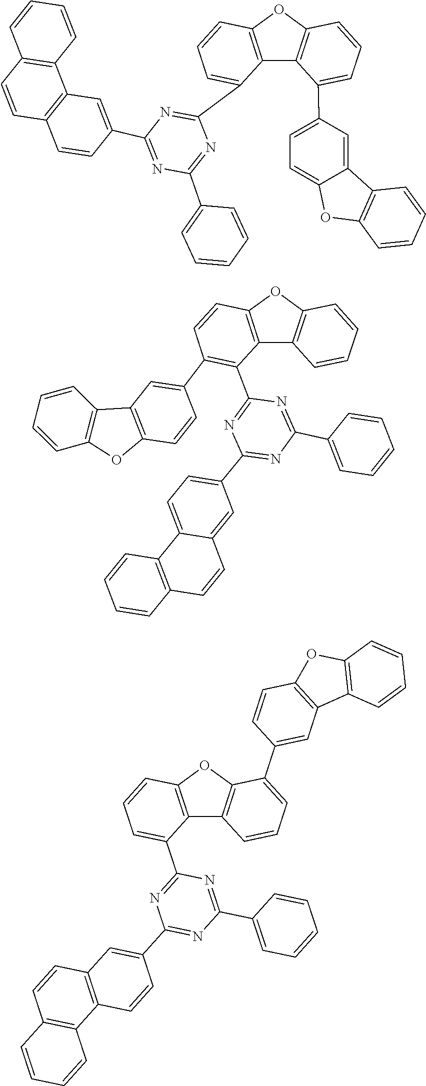

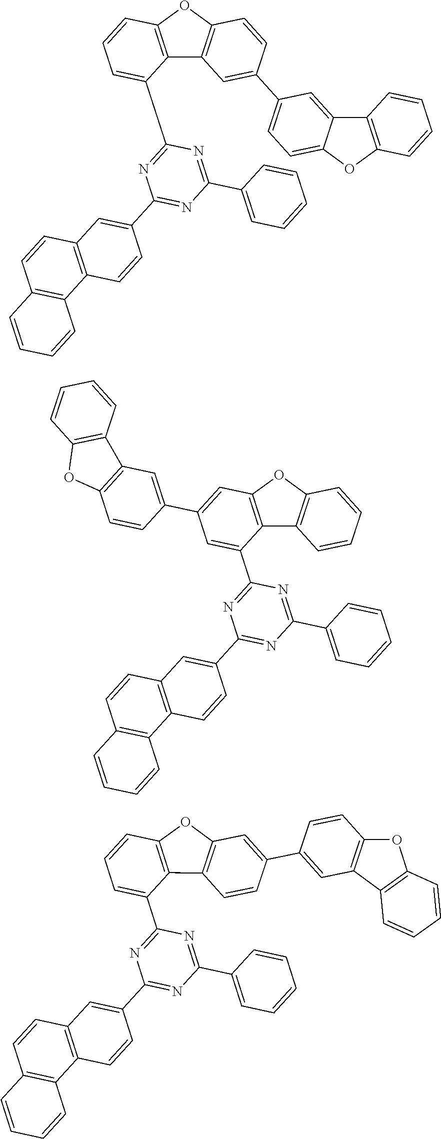

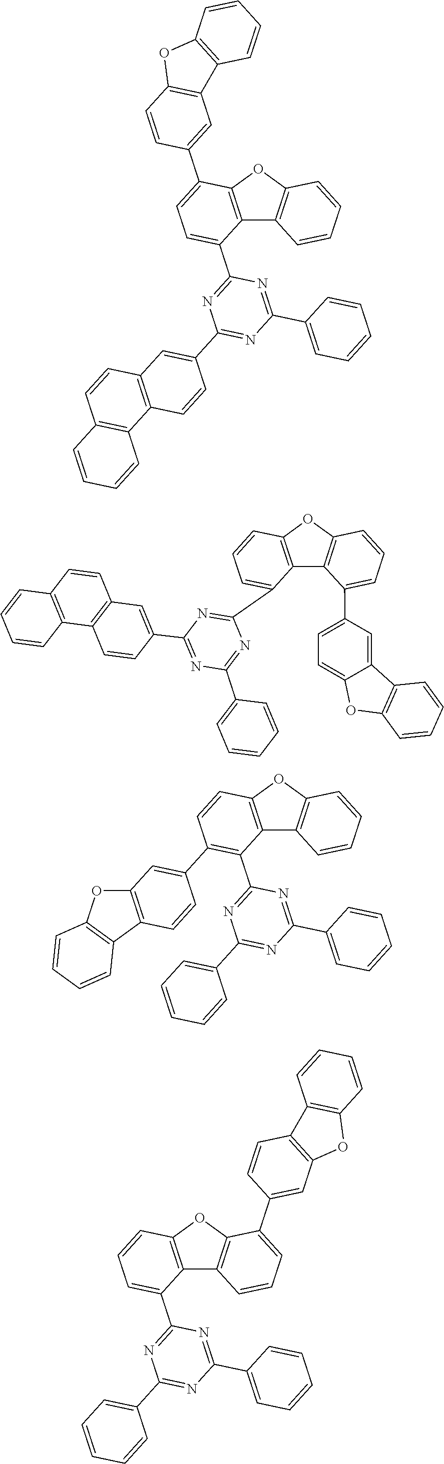

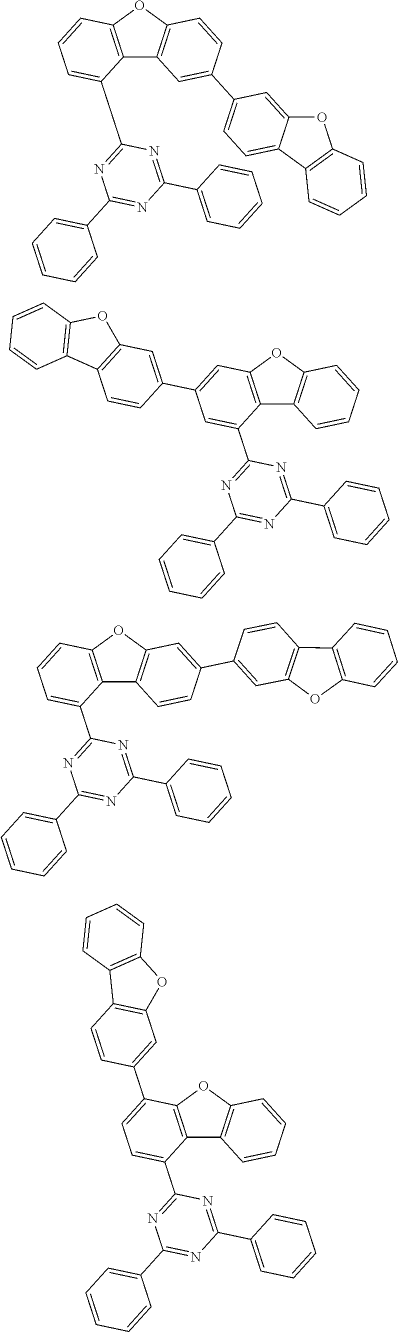

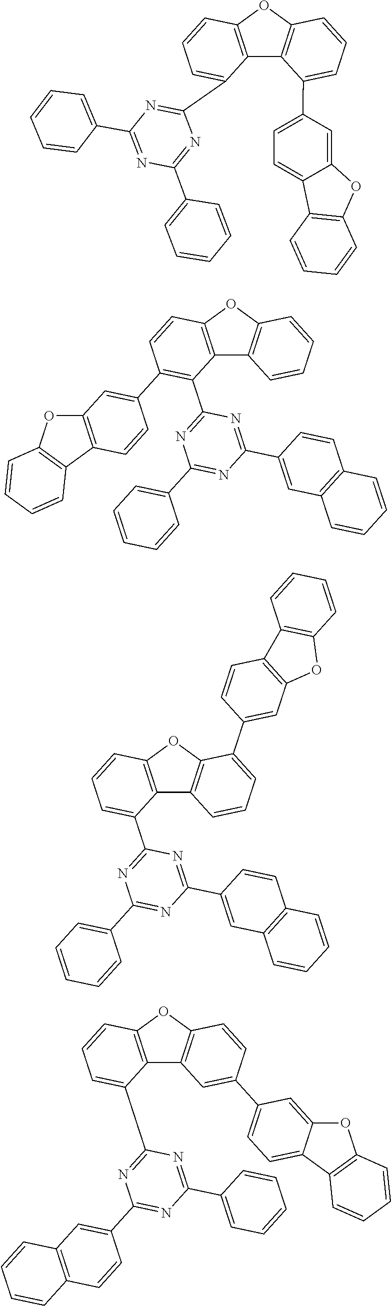

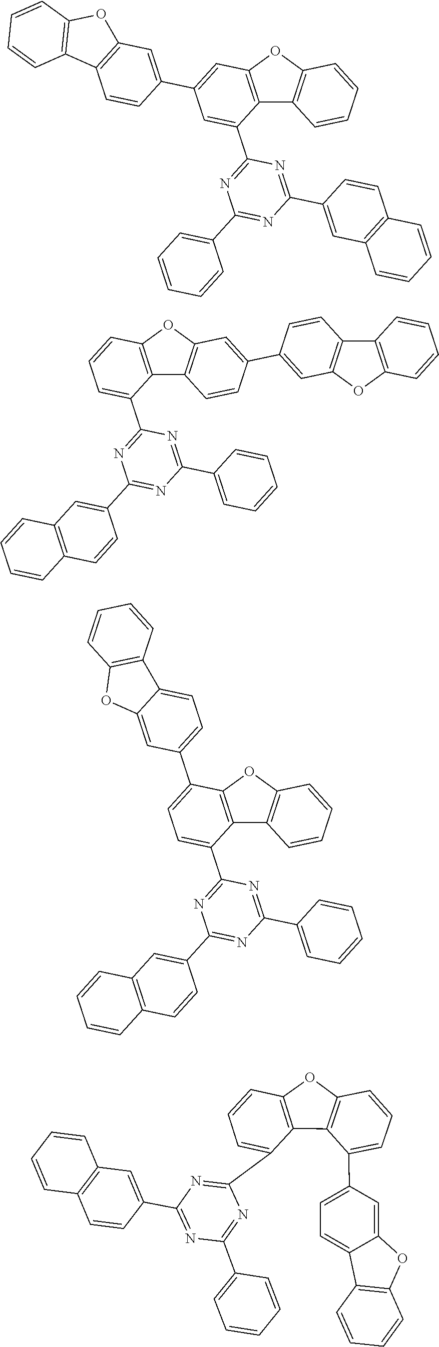

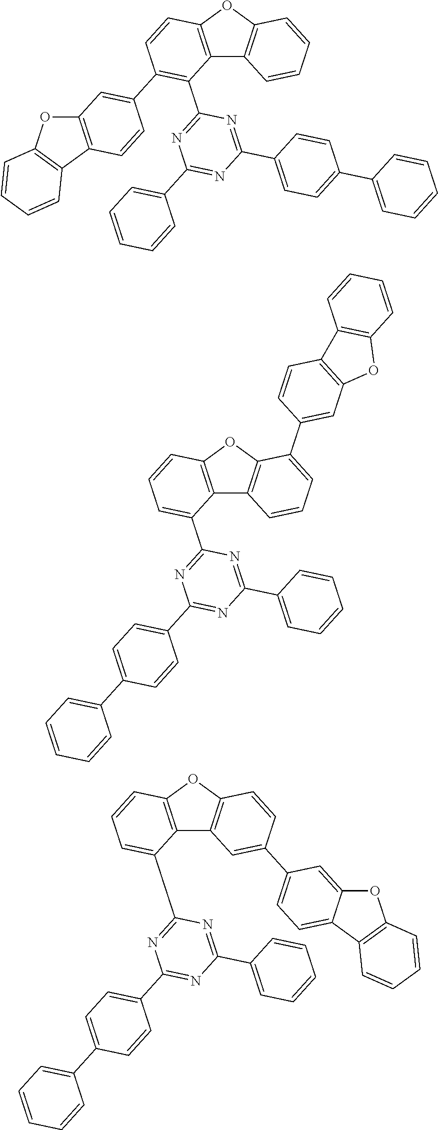 [SMILES: c1ccc(-c2ccc(-c3nc(-c4ccccc4)nc(-c4c(-c5ccc6c(c5)oc5ccccc56)ccc5oc6ccccc6c45)n3)cc2)cc1 ; c1ccc(-c2ccc(-c3nc(-c4ccccc4)nc(-c4cccc5oc6c(-c7ccc8c(c7)oc7ccccc78)cccc6c45)n3)cc2)cc1 ; c1ccc(-c2ccc(-c3nc(-c4ccccc4)nc(-c4cccc5oc6ccc(-c7ccc8c(c7)oc7ccccc78)cc6c45)n3)cc2)cc1]

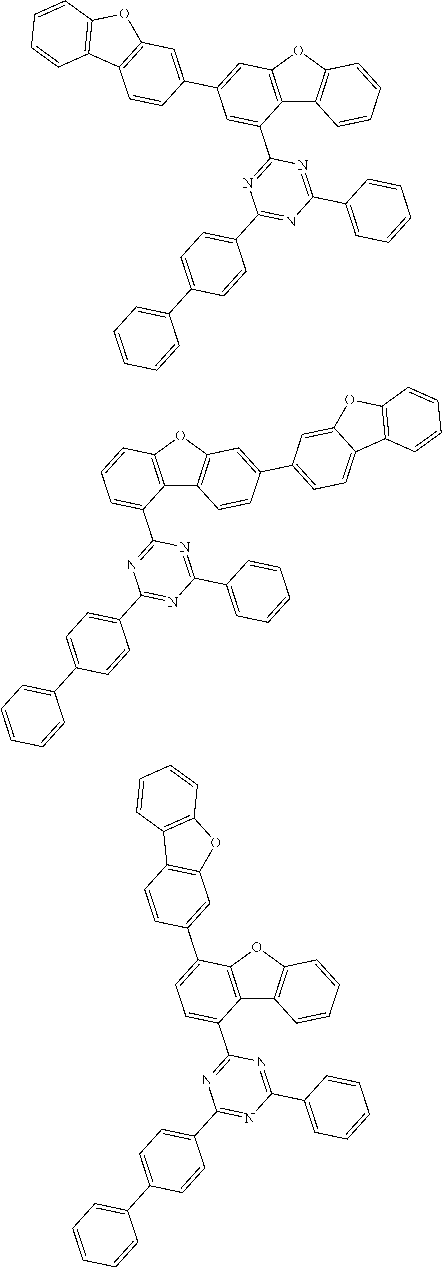 [SMILES: c1ccc(-c2ccc(-c3nc(-c4ccccc4)nc(-c4cc(-c5ccc6c(c5)oc5ccccc56)cc5oc6ccccc6c45)n3)cc2)cc1 ; c1ccc(-c2ccc(-c3nc(-c4ccccc4)nc(-c4cccc5oc6cc(-c7ccc8c(c7)oc7ccccc78)ccc6c45)n3)cc2)cc1 ; c1ccc(-c2ccc(-c3nc(-c4ccccc4)nc(-c4ccc(-c5ccc6c(c5)oc5ccccc56)c5oc6ccccc6c45)n3)cc2)cc1]

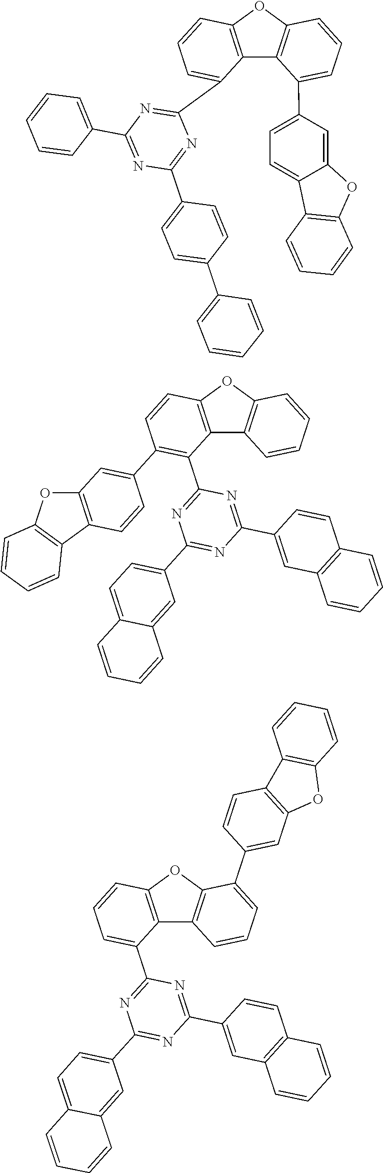

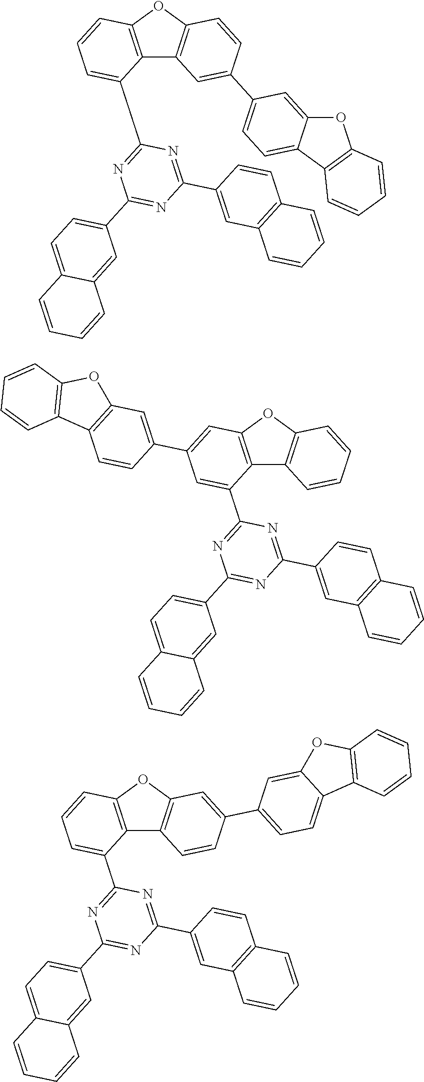

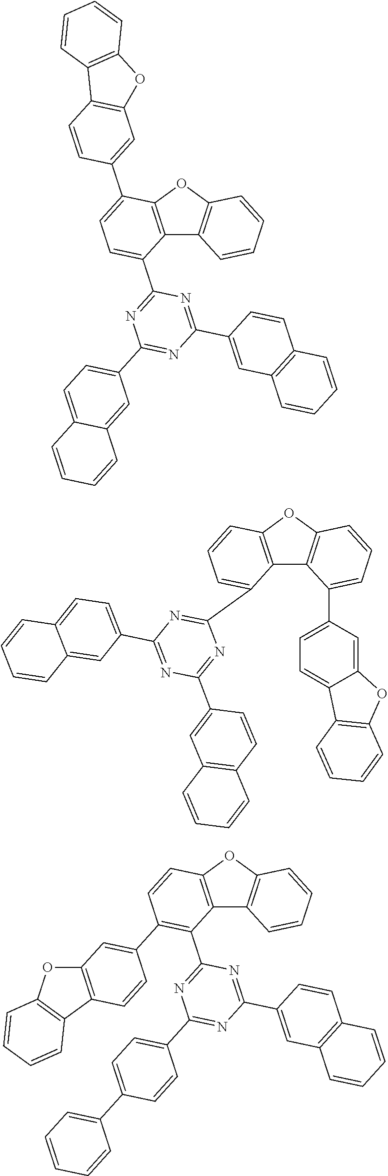

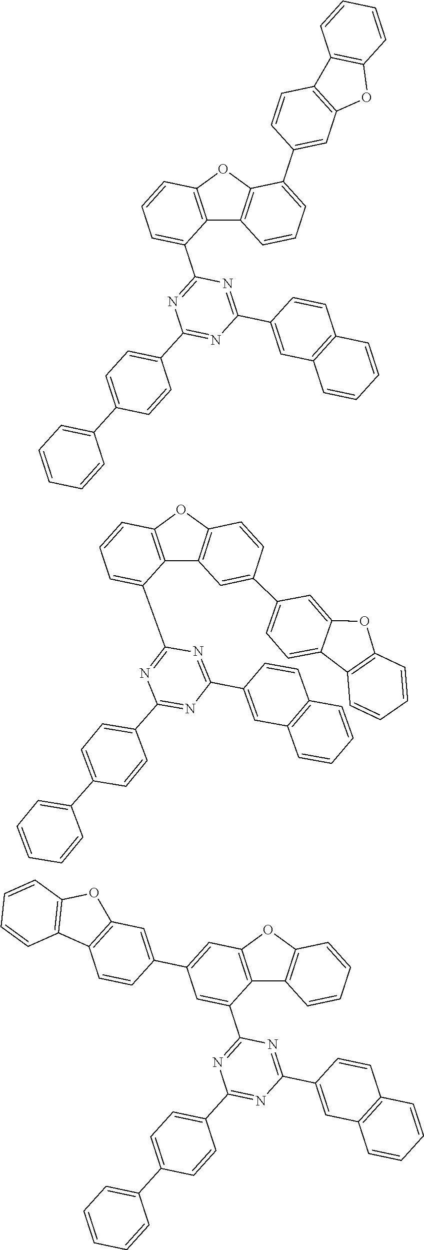

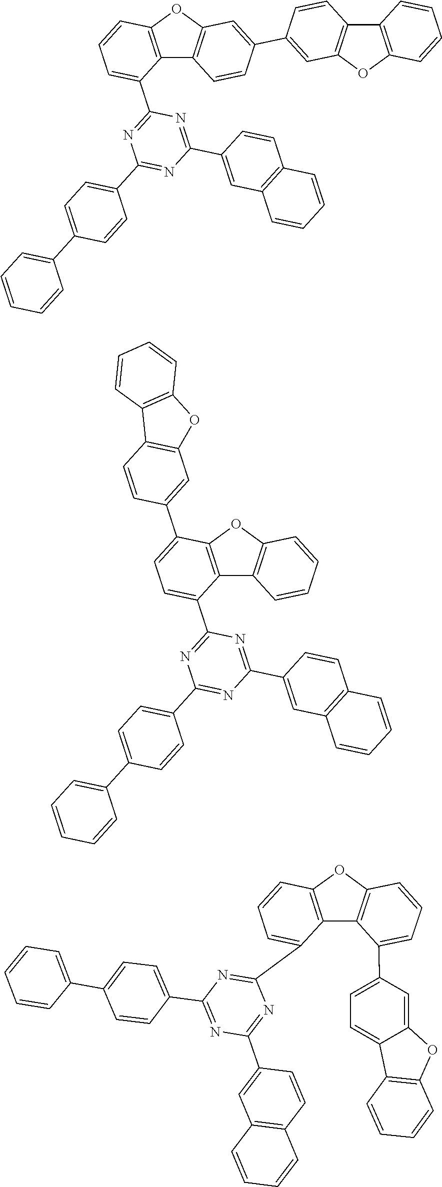 [SMILES: c1ccc(-c2ccc(-c3nc(-c4ccc5ccccc5c4)nc(-c4cccc5oc6cc(-c7ccc8c(c7)oc7ccccc78)ccc6c45)n3)cc2)cc1 ; c1ccc(-c2ccc(-c3nc(-c4ccc5ccccc5c4)nc(-c4ccc(-c5ccc6c(c5)oc5ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)oc7ccccc78)c6c45)n3)cc2)cc1]

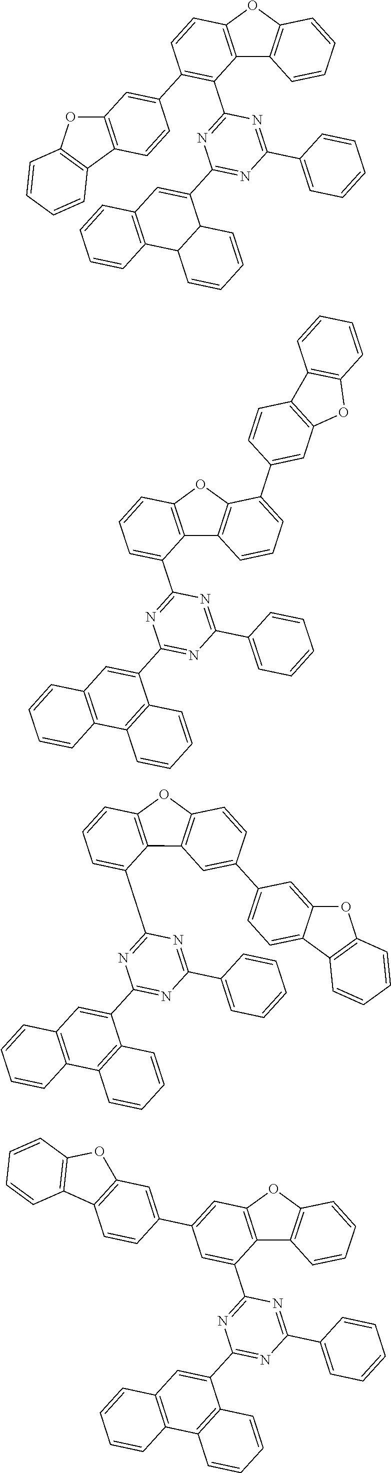

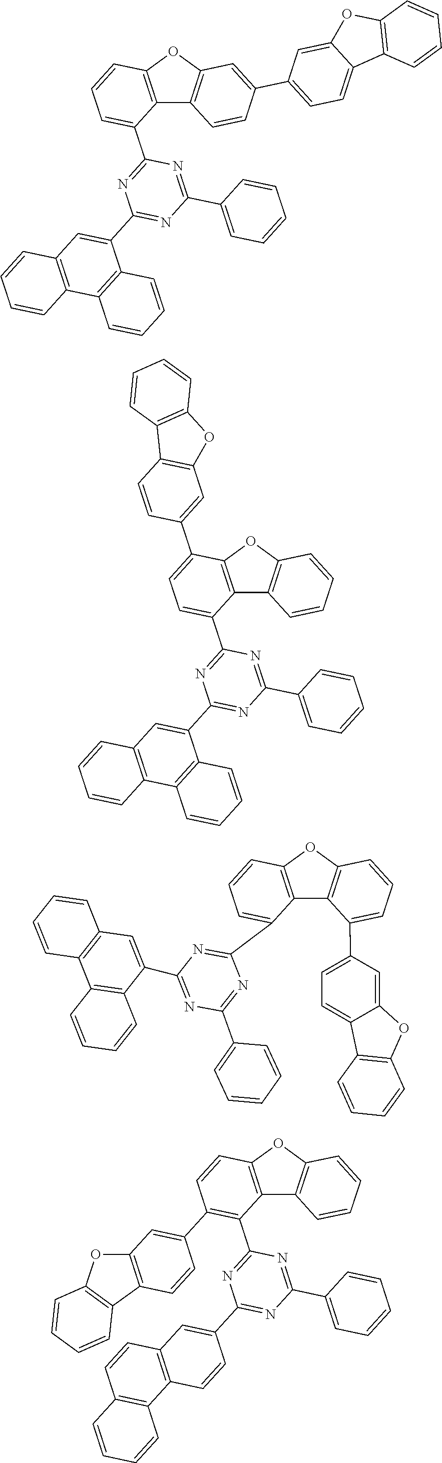

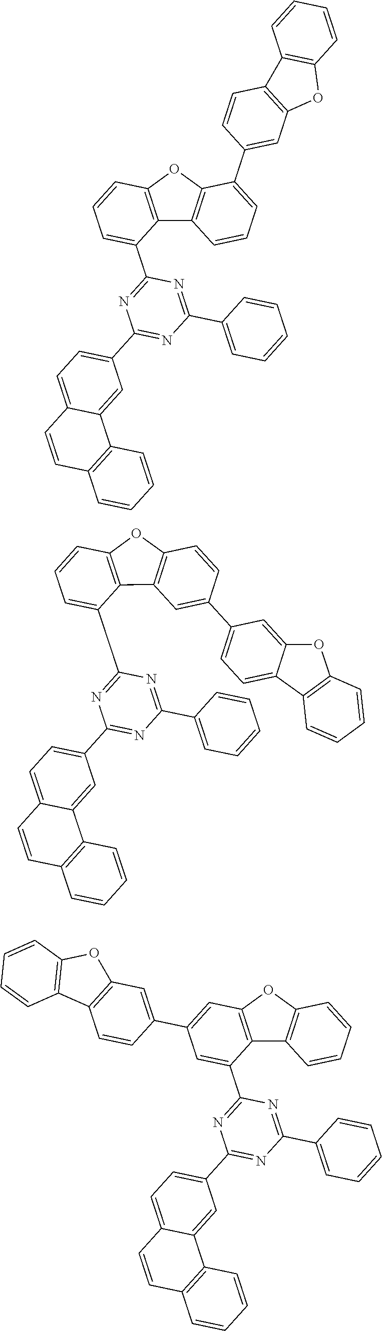

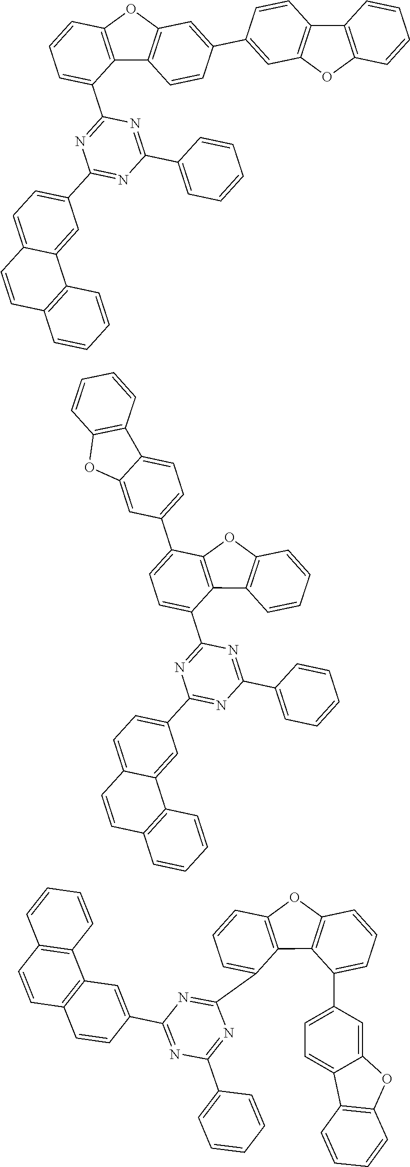

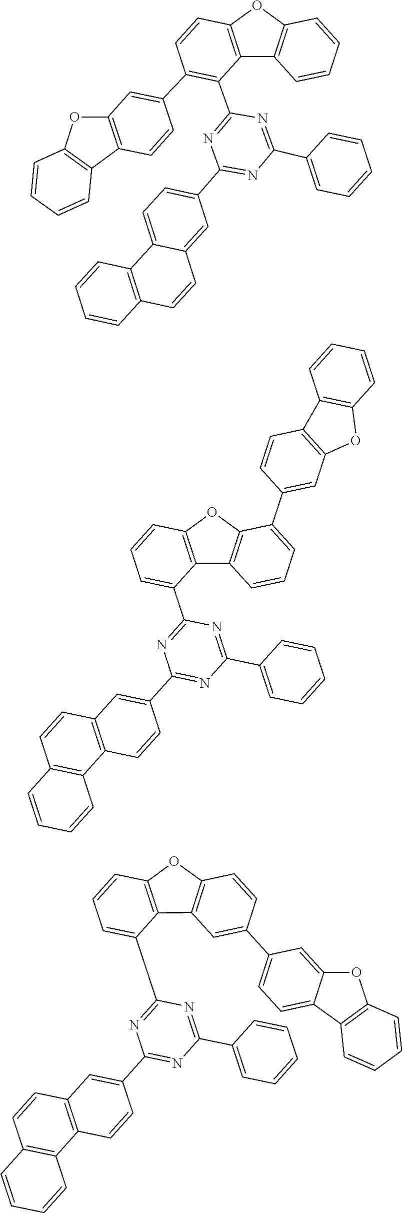 [SMILES: c1ccc(-c2nc(-c3ccc4c(ccc5ccccc54)c3)nc(-c3c(-c4ccc5c(c4)oc4ccccc45)ccc4oc5ccccc5c34)n2)cc1 ; c1ccc(-c2nc(-c3ccc4c(ccc5ccccc54)c3)nc(-c3cccc4oc5c(-c6ccc7c(c6)oc6ccccc67)cccc5c34)n2)cc1 ; c1ccc(-c2nc(-c3ccc4c(ccc5ccccc54)c3)nc(-c3cccc4oc5ccc(-c6ccc7c(c6)oc6ccccc67)cc5c34)n2)cc1]

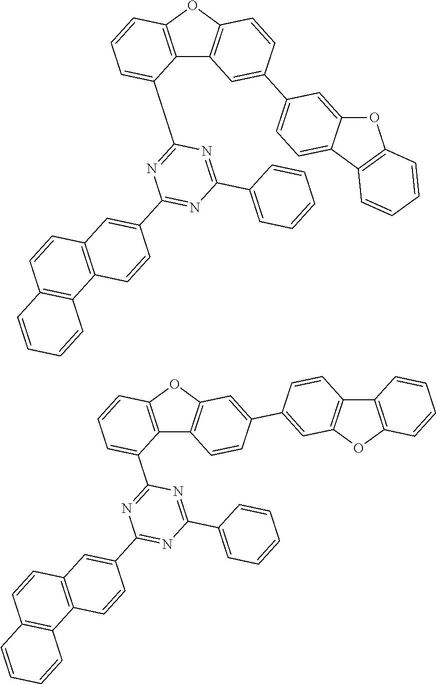 [SMILES: c1ccc(-c2nc(-c3ccc4c(ccc5ccccc54)c3)nc(-c3cccc4oc5ccc(-c6ccc7c(c6)oc6ccccc67)cc5c34)n2)cc1 ; c1ccc(-c2nc(-c3ccc4c(ccc5ccccc54)c3)nc(-c3cccc4oc5cc(-c6ccc7c(c6)oc6ccccc67)ccc5c34)n2)cc1]

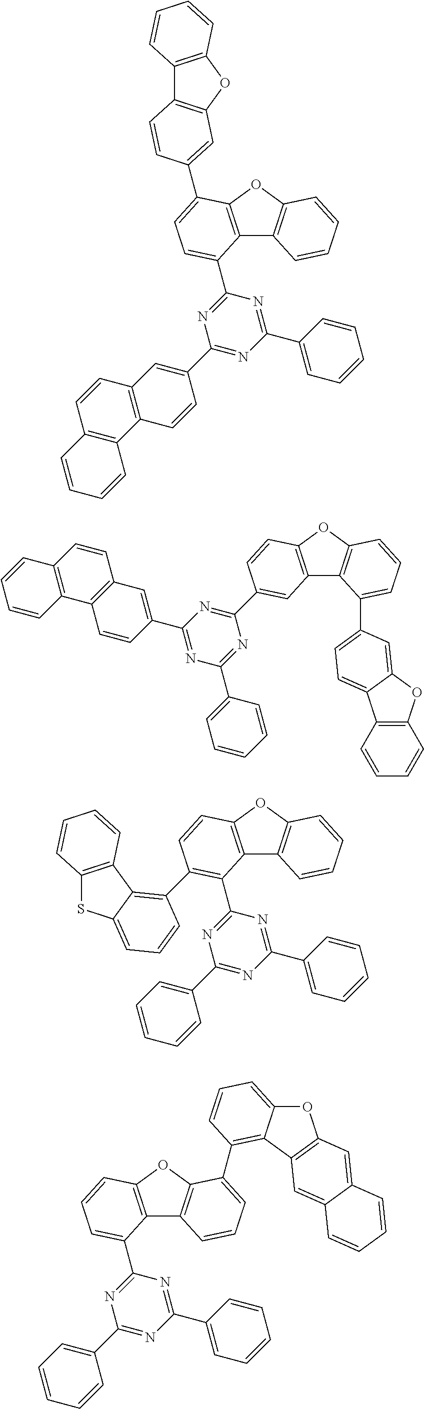 [SMILES: c1ccc(-c2nc(-c3ccc4c(ccc5ccccc54)c3)nc(-c3ccc(-c4ccc5c(c4)oc4ccccc45)c4oc5ccccc5c34)n2)cc1 ; c1ccc(-c2nc(-c3ccc4c(ccc5ccccc54)c3)nc(-c3ccc4oc5cccc(-c6ccc7c(c6)oc6ccccc67)c5c4c3)n2)cc1 ; c1ccc(-c2nc(-c3ccccc3)nc(-c3c(-c4cccc5sc6ccccc6c45)ccc4oc5ccccc5c34)n2)cc1 ; c1ccc(-c2nc(-c3ccccc3)nc(-c3cccc4oc5c(-c6cccc7oc8cc9ccccc9cc8c67)cccc5c34)n2)cc1]

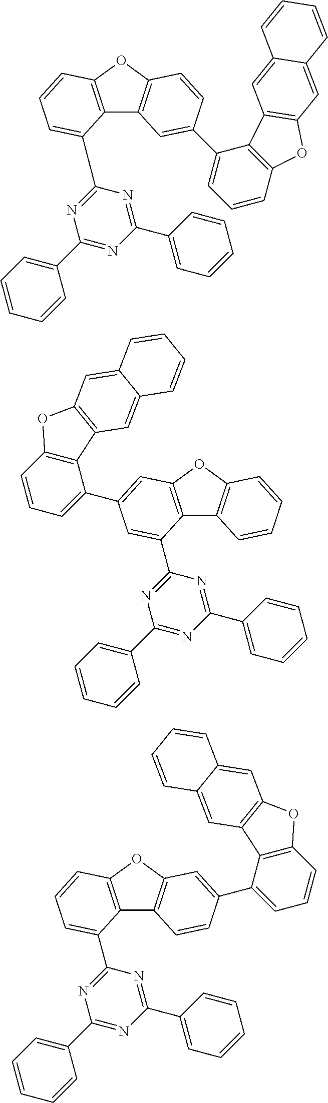 [SMILES: c1ccc(-c2nc(-c3ccccc3)nc(-c3cccc4oc5ccc(-c6cccc7oc8cc9ccccc9cc8c67)cc5c34)n2)cc1 ; c1ccc(-c2nc(-c3ccccc3)nc(-c3cc(-c4cccc5oc6cc7ccccc7cc6c45)cc4oc5ccccc5c34)n2)cc1 ; c1ccc(-c2nc(-c3ccccc3)nc(-c3cccc4oc5cc(-c6cccc7oc8cc9ccccc9cc8c67)ccc5c34)n2)cc1]

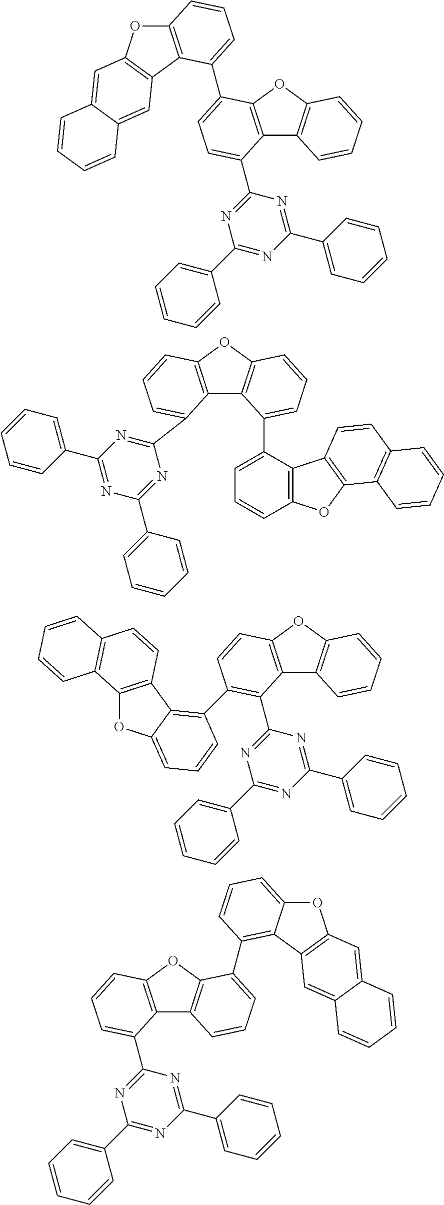

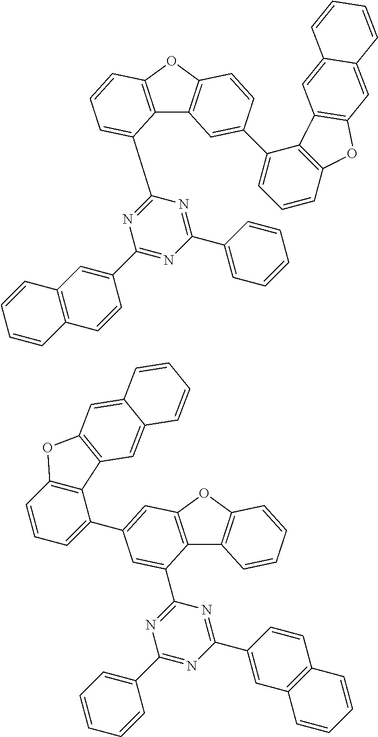 [SMILES: c1ccc(-c2nc(-c3ccc4ccccc4c3)nc(-c3cccc4oc5ccc(-c6cccc7oc8cc9ccccc9cc8c67)cc5c34)n2)cc1 ; c1ccc(-c2nc(-c3ccc4ccccc4c3)nc(-c3cc(-c4cccc5oc6cc7ccccc7cc6c45)cc4oc5ccccc5c34)n2)cc1]

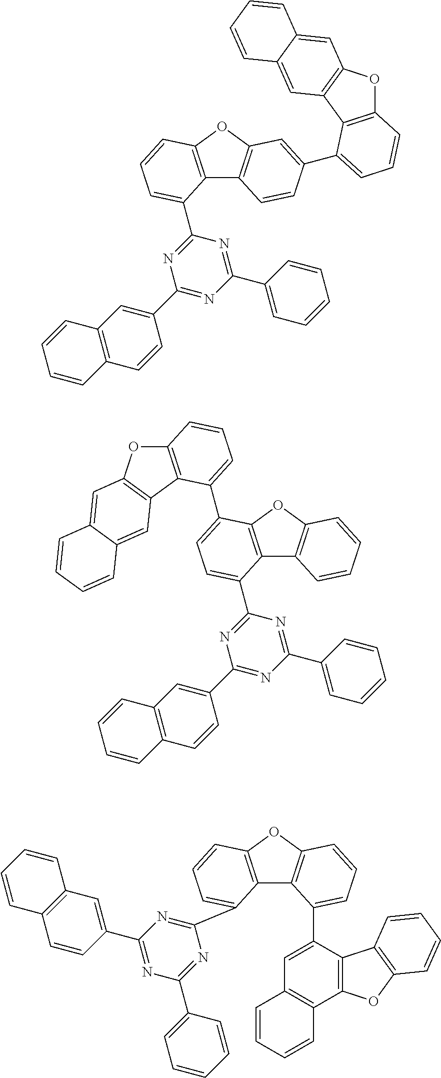 [SMILES: c1ccc(-c2nc(-c3ccc4ccccc4c3)nc(-c3cccc4oc5cc(-c6cccc7oc8cc9ccccc9cc8c67)ccc5c34)n2)cc1 ; c1ccc(-c2nc(-c3ccc4ccccc4c3)nc(-c3ccc(-c4cccc5oc6cc7ccccc7cc6c45)c4oc5ccccc5c34)n2)cc1 ; c1ccc(-c2nc(-c3ccc4ccccc4c3)nc(-c3cccc4oc5cccc(-c6cc7ccccc7c7oc8ccccc8c67)c5c34)n2)cc1]

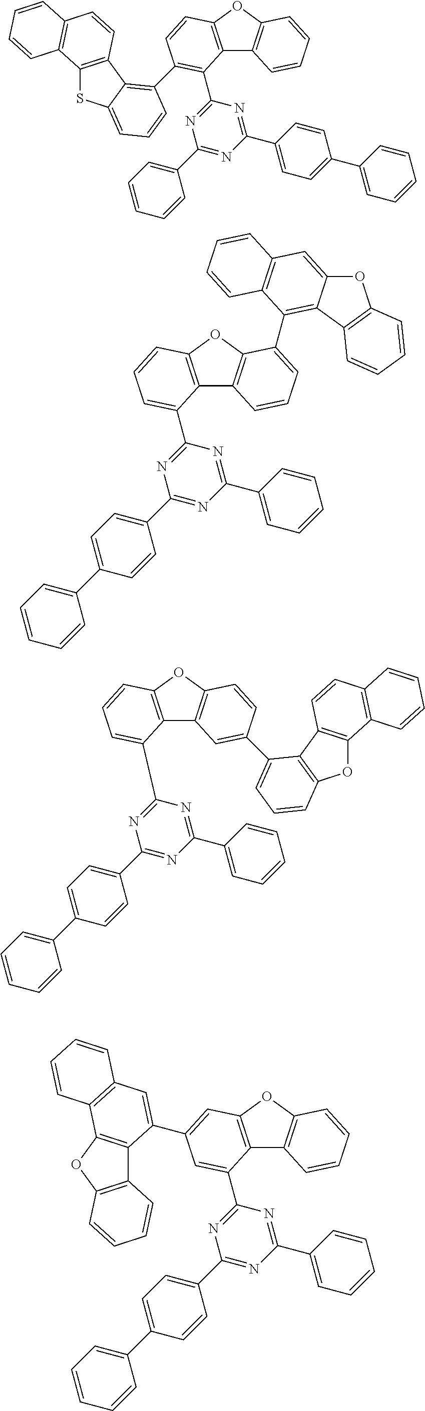 [SMILES: c1ccc(-c2ccc(-c3nc(-c4ccccc4)nc(-c4c(-c5cccc6sc7c8ccccc8ccc7c56)ccc5oc6ccccc6c45)n3)cc2)cc1 ; c1ccc(-c2ccc(-c3nc(-c4ccccc4)nc(-c4cccc5oc6c(-c7c8ccccc8cc8oc9ccccc9c78)cccc6c45)n3)cc2)cc1 ; c1ccc(-c2ccc(-c3nc(-c4ccccc4)nc(-c4cccc5oc6ccc(-c7cccc8oc9c%10ccccc%10ccc9c78)cc6c45)n3)cc2)cc1 ; c1ccc(-c2ccc(-c3nc(-c4ccccc4)nc(-c4cc(-c5cc6ccccc6c6oc7ccccc7c56)cc5oc6ccccc6c45)n3)cc2)cc1]

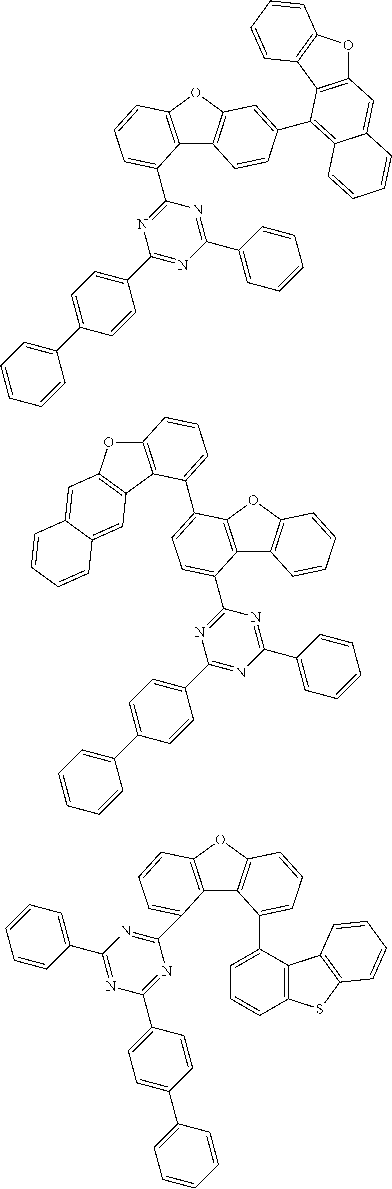 [SMILES: c1ccc(-c2ccc(-c3nc(-c4ccccc4)nc(-c4cccc5oc6cc(-c7c8ccccc8cc8oc9ccccc9c78)ccc6c45)n3)cc2)cc1 ; c1ccc(-c2ccc(-c3nc(-c4ccccc4)nc(-c4ccc(-c5cccc6oc7cc8ccccc8cc7c56)c5oc6ccccc6c45)n3)cc2)cc1 ; c1ccc(-c2ccc(-c3nc(-c4ccccc4)nc(-c4cccc5oc6cccc(-c7cccc8sc9ccccc9c78)c6c45)n3)cc2)cc1]

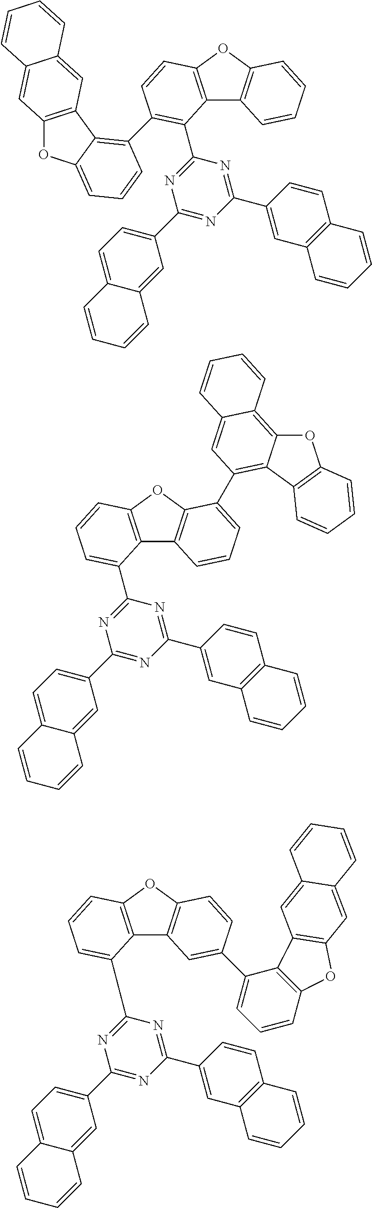 [SMILES: c1ccc2cc(-c3nc(-c4ccc5ccccc5c4)nc(-c4c(-c5cccc6oc7cc8ccccc8cc7c56)ccc5oc6ccccc6c45)n3)ccc2c1 ; c1ccc2cc(-c3nc(-c4ccc5ccccc5c4)nc(-c4cccc5oc6c(-c7cc8ccccc8c8oc9ccccc9c78)cccc6c45)n3)ccc2c1 ; c1ccc2cc(-c3nc(-c4ccc5ccccc5c4)nc(-c4cccc5oc6ccc(-c7cccc8oc9cc%10ccccc%10cc9c78)cc6c45)n3)ccc2c1]

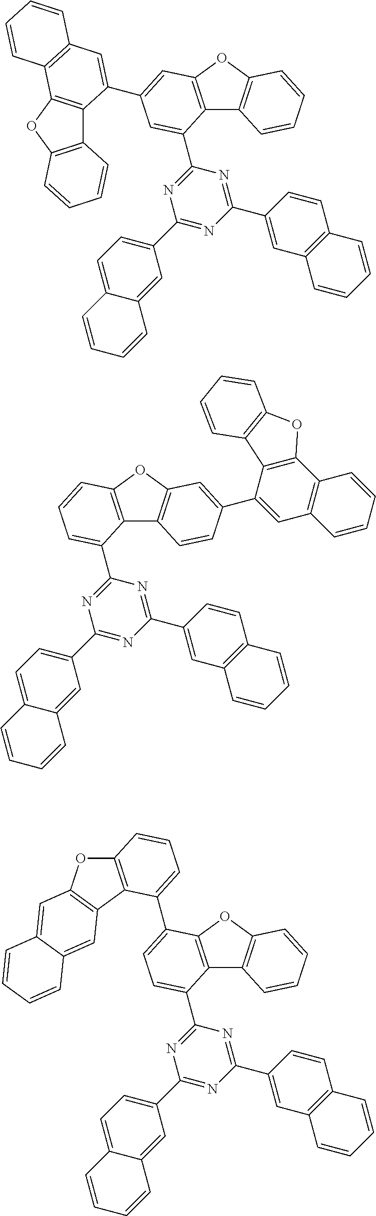 [SMILES: c1ccc2cc(-c3nc(-c4ccc5ccccc5c4)nc(-c4cc(-c5cc6ccccc6c6oc7ccccc7c56)cc5oc6ccccc6c45)n3)ccc2c1 ; c1ccc2cc(-c3nc(-c4ccc5ccccc5c4)nc(-c4cccc5oc6cc(-c7cc8ccccc8c8oc9ccccc9c78)ccc6c45)n3)ccc2c1 ; c1ccc2cc(-c3nc(-c4ccc5ccccc5c4)nc(-c4ccc(-c5cccc6oc7cc8ccccc8cc7c56)c5oc6ccccc6c45)n3)ccc2c1]

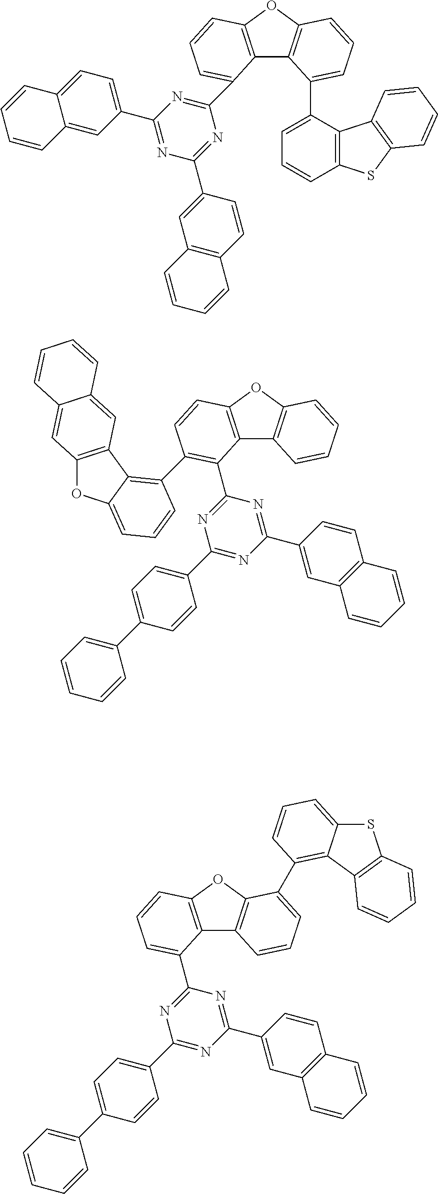 [SMILES: c1ccc2cc(-c3nc(-c4ccc5ccccc5c4)nc(-c4cccc5oc6cccc(-c7cccc8sc9ccccc9c78)c6c45)n3)ccc2c1 ; c1ccc(-c2ccc(-c3nc(-c4ccc5ccccc5c4)nc(-c4c(-c5cccc6oc7cc8ccccc8cc7c56)ccc5oc6ccccc6c45)n3)cc2)cc1 ; c1ccc(-c2ccc(-c3nc(-c4ccc5ccccc5c4)nc(-c4cccc5oc6c(-c7cccc8sc9ccccc9c78)cccc6c45)n3)cc2)cc1]

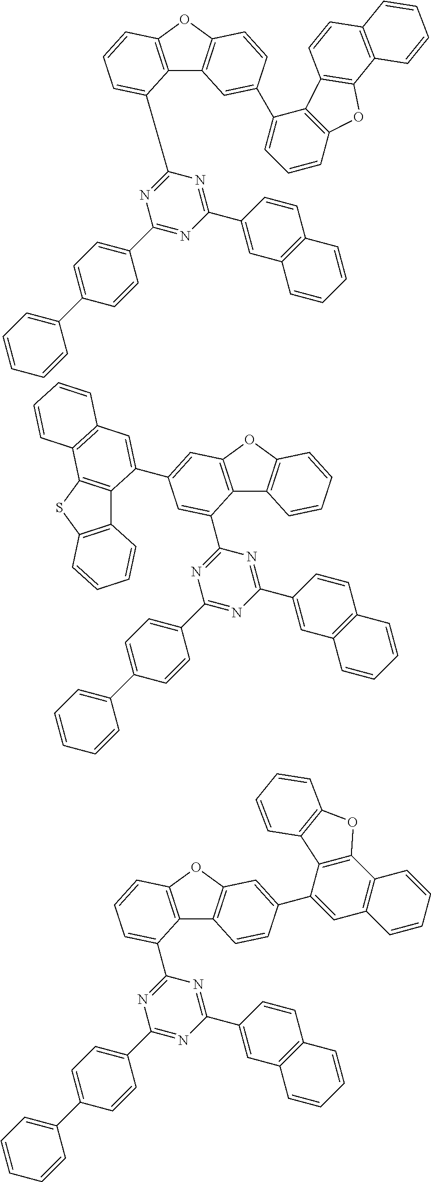

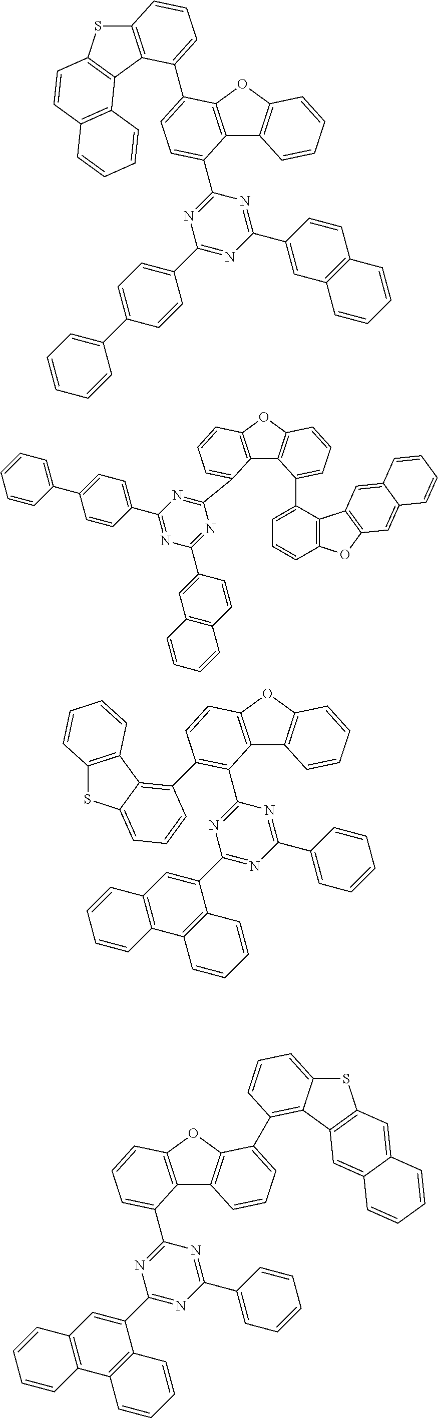

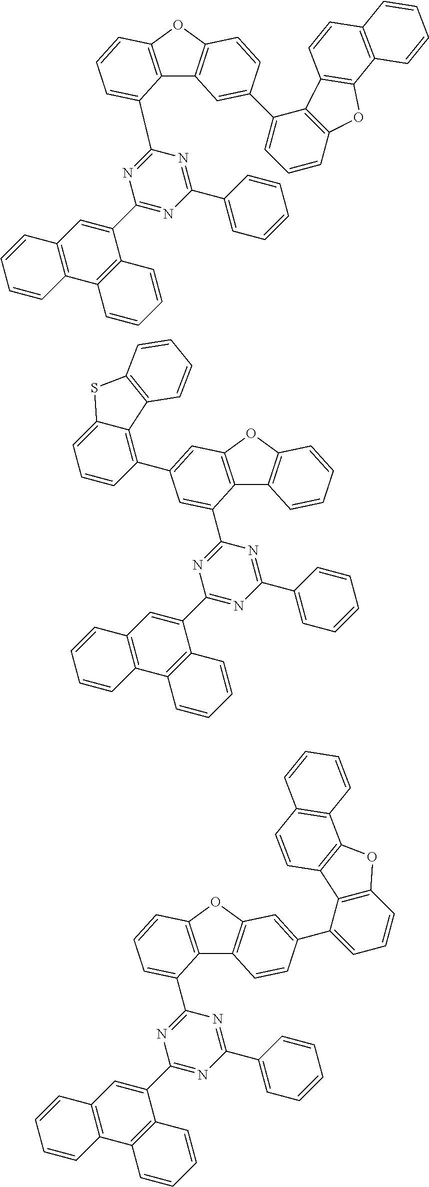

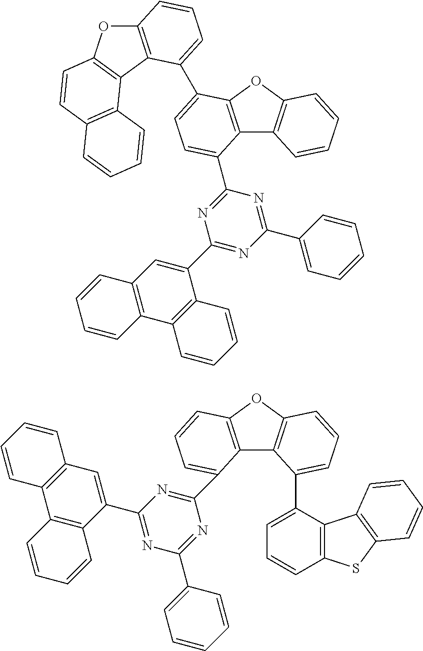 [SMILES: c1ccc(-c2nc(-c3cc4ccccc4c4ccccc34)nc(-c3ccc(-c4cccc5oc6ccc7ccccc7c6c45)c4oc5ccccc5c34)n2)cc1 ; c1ccc(-c2nc(-c3cc4ccccc4c4ccccc34)nc(-c3cccc4oc5cccc(-c6cccc7sc8ccccc8c67)c5c34)n2)cc1]

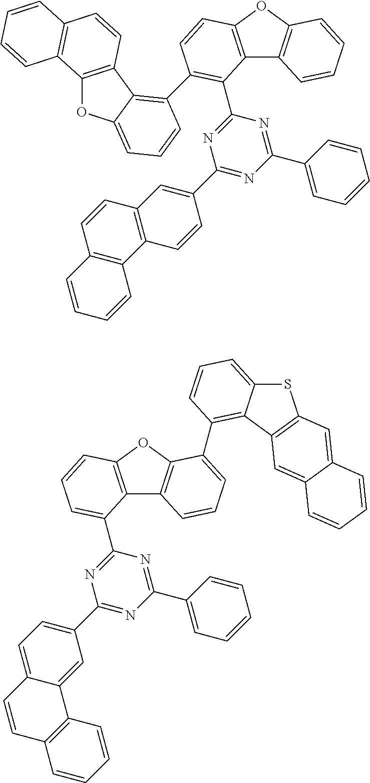 [SMILES: c1ccc(-c2nc(-c3ccc4c(ccc5ccccc54)c3)nc(-c3c(-c4cccc5oc6c7ccccc7ccc6c45)ccc4oc5ccccc5c34)n2)cc1 ; c1ccc(-c2nc(-c3ccc4ccc5ccccc5c4c3)nc(-c3cccc4oc5c(-c6cccc7sc8cc9ccccc9cc8c67)cccc5c34)n2)cc1]

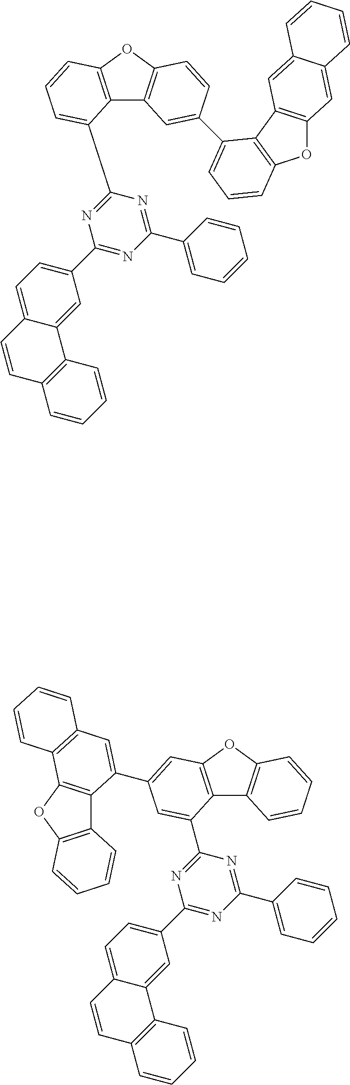 [SMILES: c1ccc(-c2nc(-c3ccc4ccc5ccccc5c4c3)nc(-c3cccc4oc5ccc(-c6cccc7oc8cc9ccccc9cc8c67)cc5c34)n2)cc1 ; c1ccc(-c2nc(-c3ccc4ccc5ccccc5c4c3)nc(-c3cc(-c4cc5ccccc5c5oc6ccccc6c45)cc4oc5ccccc5c34)n2)cc1]

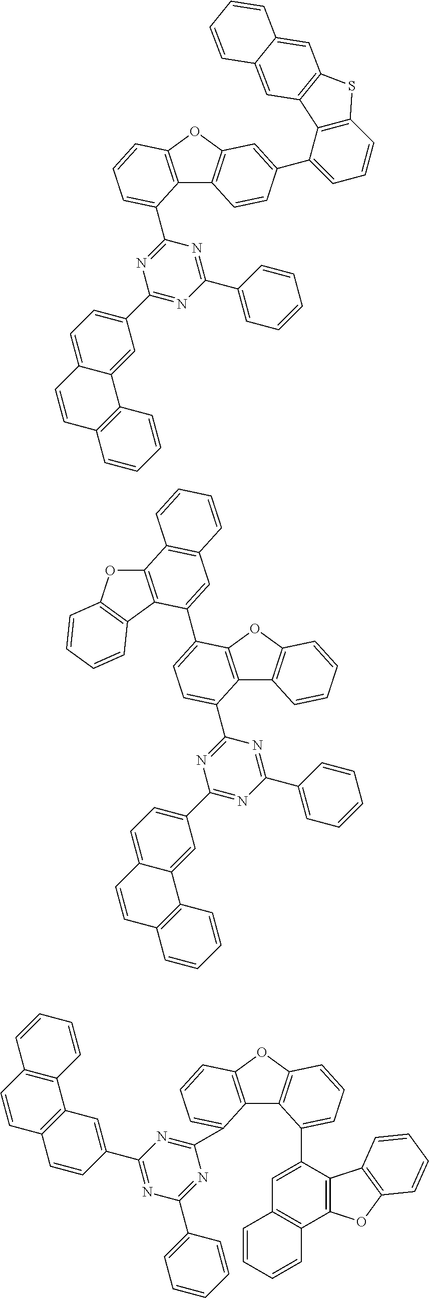

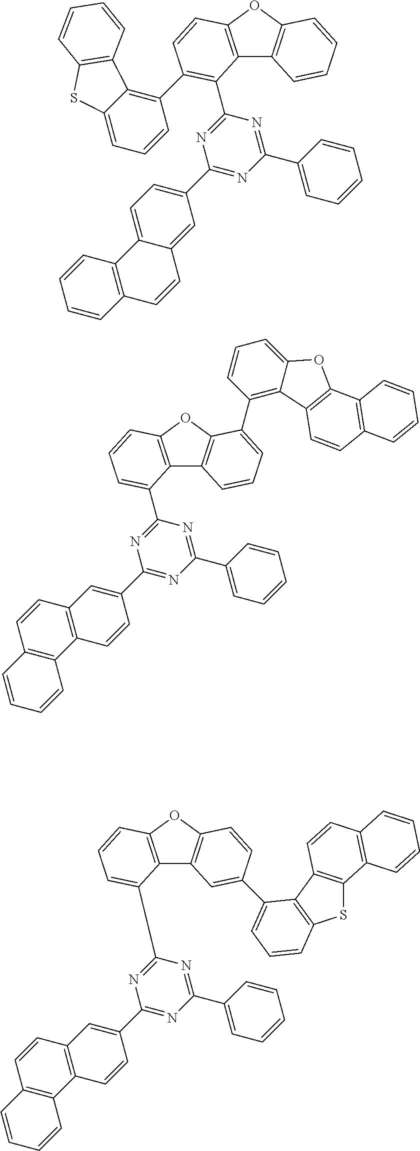 [SMILES: c1ccc(-c2nc(-c3ccc4c(ccc5ccccc54)c3)nc(-c3c(-c4cccc5sc6ccccc6c45)ccc4oc5ccccc5c34)n2)cc1 ; c1ccc(-c2nc(-c3ccc4c(ccc5ccccc54)c3)nc(-c3cccc4oc5c(-c6cccc7oc8c9ccccc9ccc8c67)cccc5c34)n2)cc1 ; c1ccc(-c2nc(-c3ccc4c(ccc5ccccc54)c3)nc(-c3cccc4oc5ccc(-c6cccc7sc8c9ccccc9ccc8c67)cc5c34)n2)cc1]

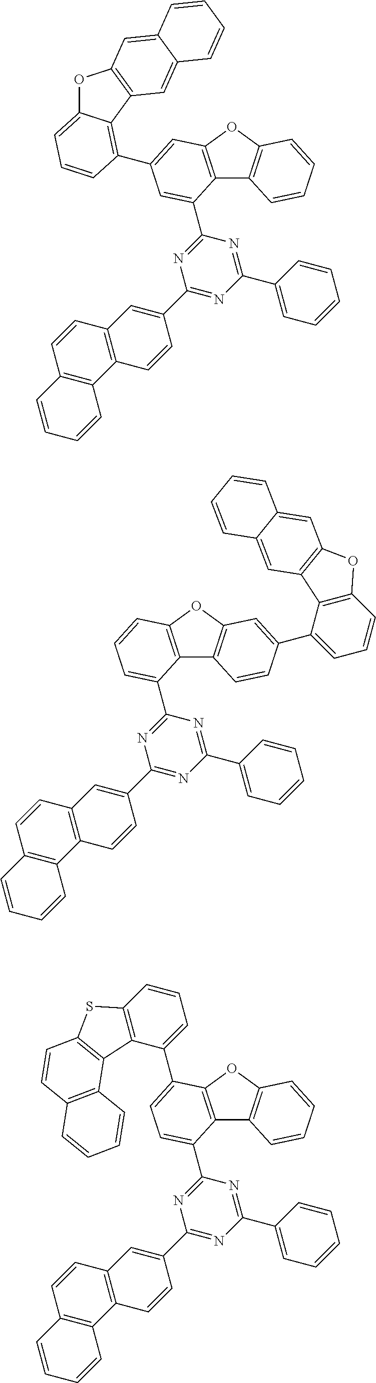 [SMILES: c1ccc(-c2nc(-c3ccc4c(ccc5ccccc54)c3)nc(-c3cc(-c4cccc5oc6cc7ccccc7cc6c45)cc4oc5ccccc5c34)n2)cc1 ; c1ccc(-c2nc(-c3ccc4c(ccc5ccccc54)c3)nc(-c3cccc4oc5cc(-c6cccc7oc8cc9ccccc9cc8c67)ccc5c34)n2)cc1 ; c1ccc(-c2nc(-c3ccc4c(ccc5ccccc54)c3)nc(-c3ccc(-c4cccc5sc6ccc7ccccc7c6c45)c4oc5ccccc5c34)n2)cc1]

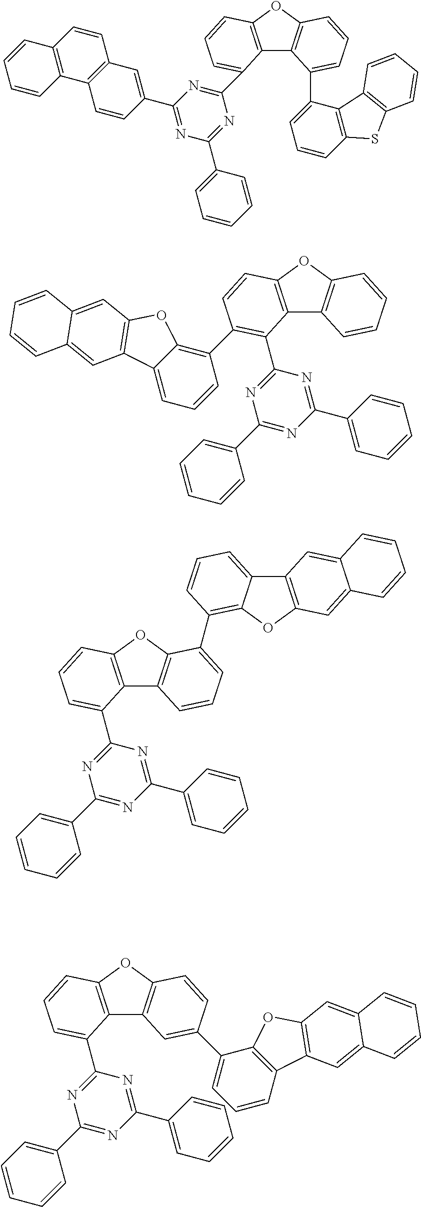 [SMILES: c1ccc(-c2nc(-c3ccc4c(ccc5ccccc54)c3)nc(-c3cccc4oc5cccc(-c6cccc7sc8ccccc8c67)c5c34)n2)cc1 ; c1ccc(-c2nc(-c3ccccc3)nc(-c3c(-c4cccc5c4oc4cc6ccccc6cc45)ccc4oc5ccccc5c34)n2)cc1 ; c1ccc(-c2nc(-c3ccccc3)nc(-c3cccc4oc5c(-c6cccc7c6oc6cc8ccccc8cc67)cccc5c34)n2)cc1 ; c1ccc(-c2nc(-c3ccccc3)nc(-c3cccc4oc5ccc(-c6cccc7c6oc6cc8ccccc8cc67)cc5c34)n2)cc1]

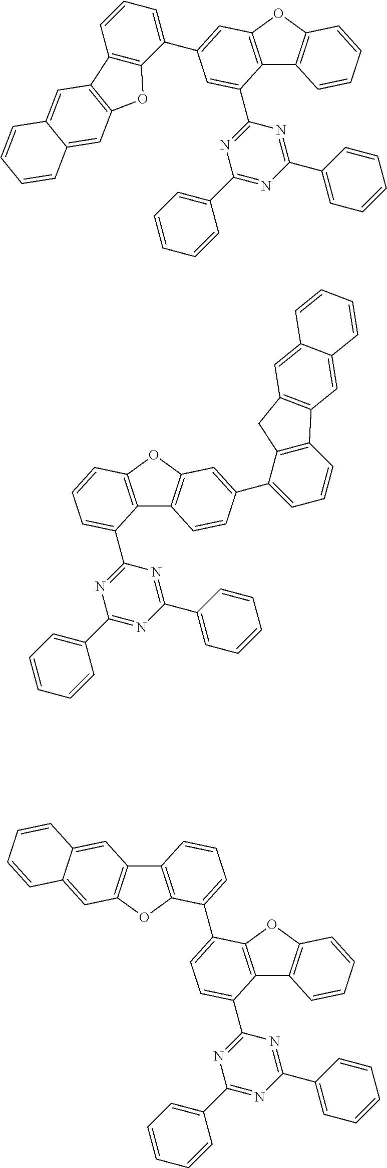 [SMILES: c1ccc(-c2nc(-c3ccccc3)nc(-c3cc(-c4cccc5c4oc4cc6ccccc6cc45)cc4oc5ccccc5c34)n2)cc1 ; c1ccc(-c2nc(-c3ccccc3)nc(-c3cccc4oc5cc(-c6cccc7c6Cc6cc8ccccc8cc6-7)ccc5c34)n2)cc1 ; c1ccc(-c2nc(-c3ccccc3)nc(-c3ccc(-c4cccc5c4oc4cc6ccccc6cc45)c4oc5ccccc5c34)n2)cc1]

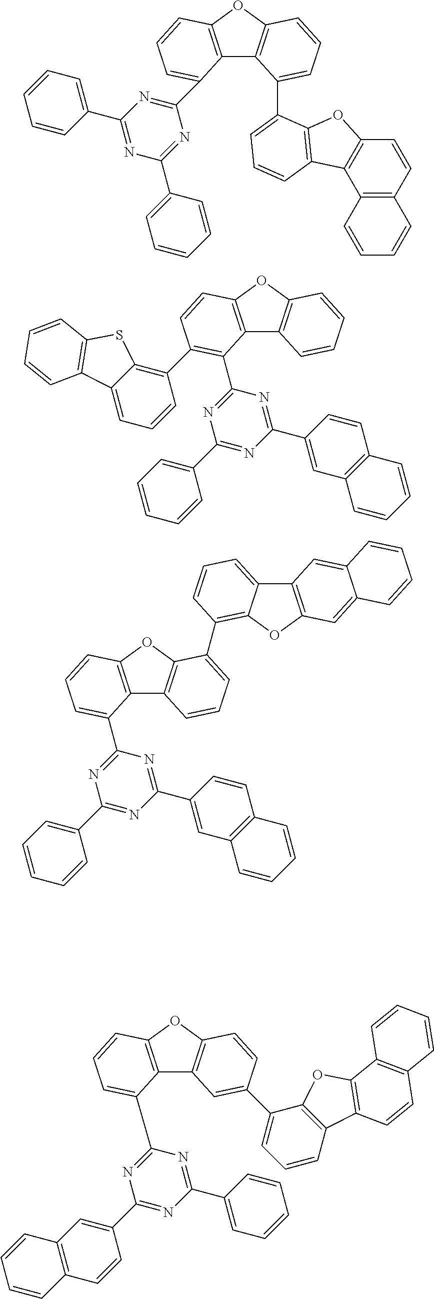

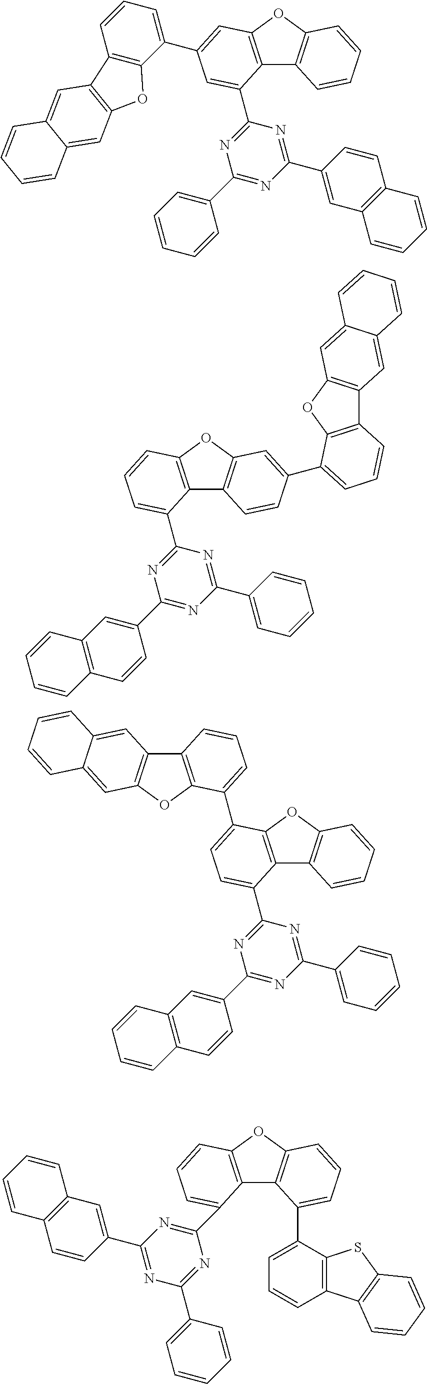

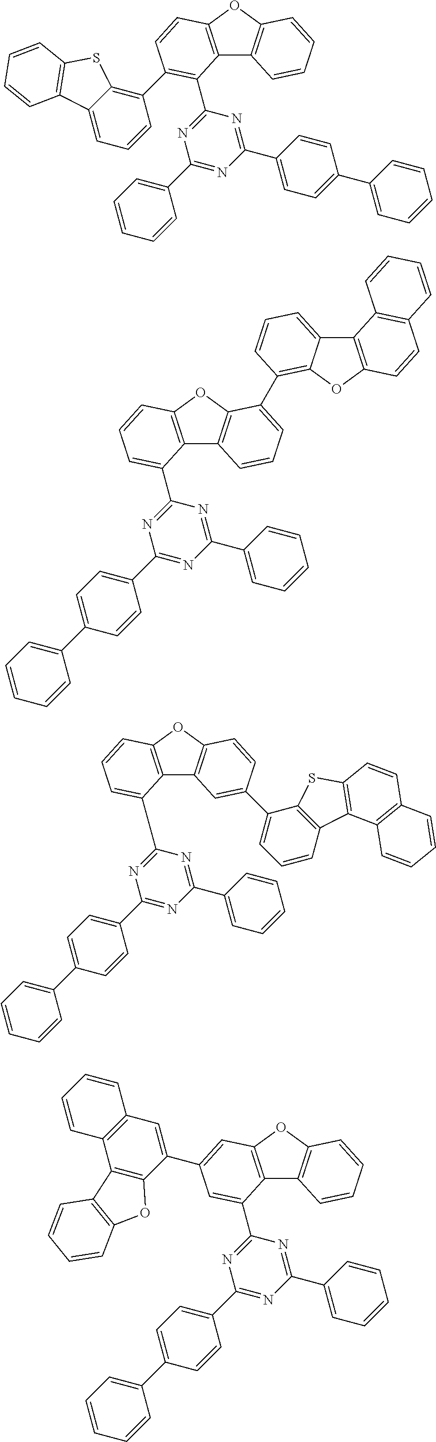

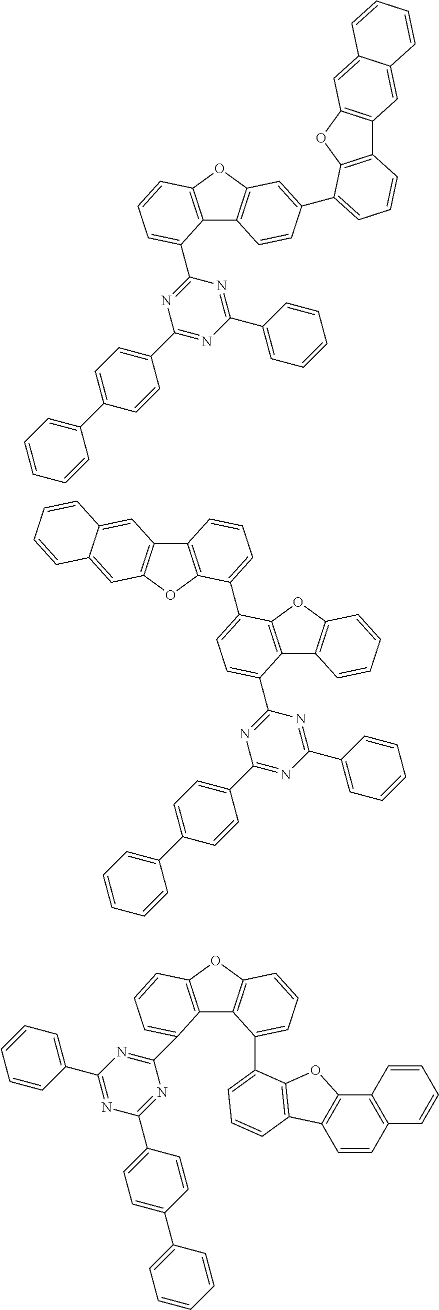

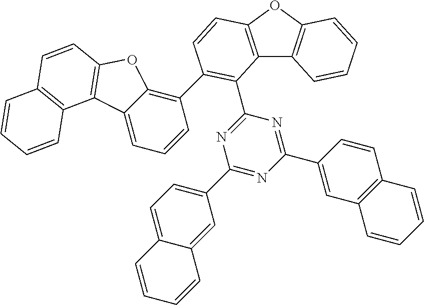 [SMILES: c1ccc2cc(-c3nc(-c4ccc5ccccc5c4)nc(-c4c(-c5cccc6c5oc5ccc7ccccc7c56)ccc5oc6ccccc6c45)n3)ccc2c1]

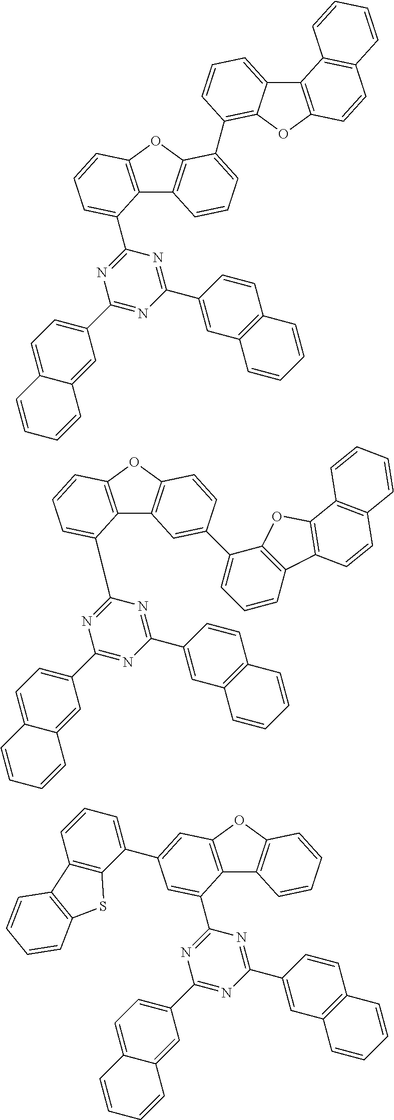

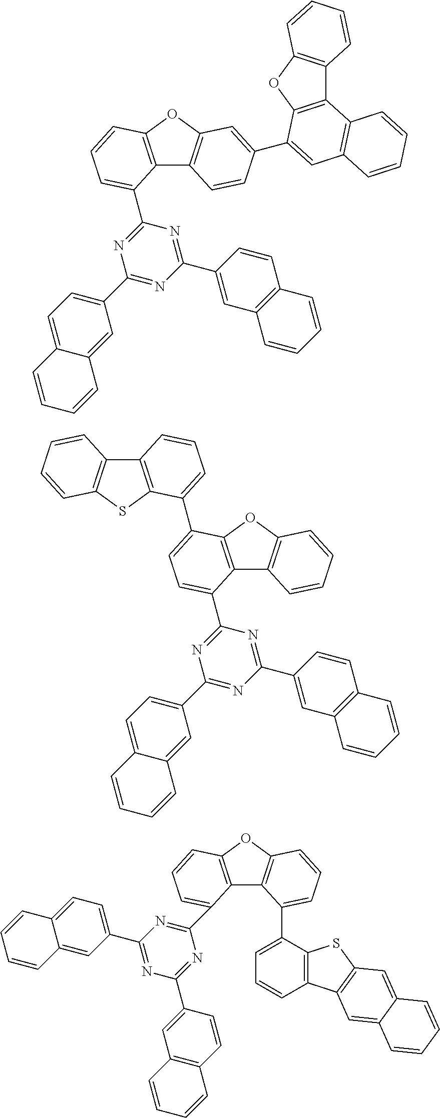

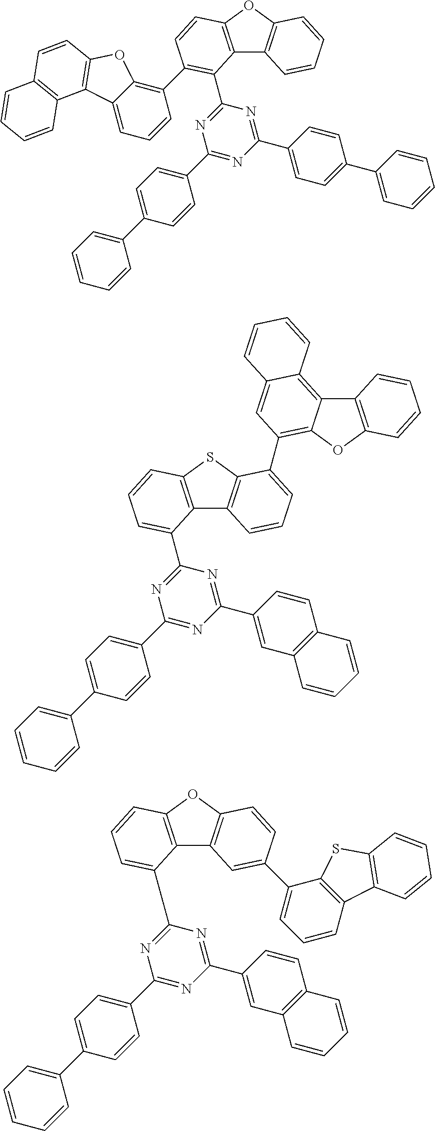 [SMILES: c1ccc(-c2ccc(-c3nc(-c4ccc(-c5ccccc5)cc4)nc(-c4c(-c5cccc6c5oc5ccc7ccccc7c56)ccc5oc6ccccc6c45)n3)cc2)cc1 ; c1ccc(-c2ccc(-c3nc(-c4ccc5ccccc5c4)nc(-c4cccc5sc6c(-c7cc8ccccc8c8c7oc7ccccc78)cccc6c45)n3)cc2)cc1 ; c1ccc(-c2ccc(-c3nc(-c4ccc5ccccc5c4)nc(-c4cccc5oc6ccc(-c7cccc8c7sc7ccccc78)cc6c45)n3)cc2)cc1]

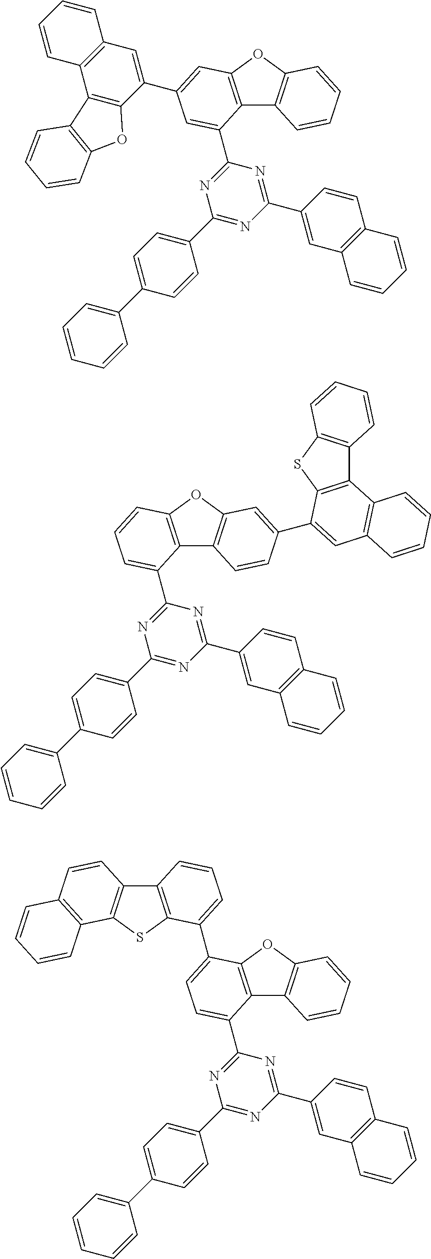 [SMILES: c1ccc(-c2ccc(-c3nc(-c4ccc5ccccc5c4)nc(-c4cc(-c5cc6ccccc6c6c5oc5ccccc56)cc5oc6ccccc6c45)n3)cc2)cc1 ; c1ccc(-c2ccc(-c3nc(-c4ccc5ccccc5c4)nc(-c4cccc5oc6cc(-c7cc8ccccc8c8c7sc7ccccc78)ccc6c45)n3)cc2)cc1 ; c1ccc(-c2ccc(-c3nc(-c4ccc5ccccc5c4)nc(-c4ccc(-c5cccc6c5sc5c7ccccc7ccc65)c5oc6ccccc6c45)n3)cc2)cc1]

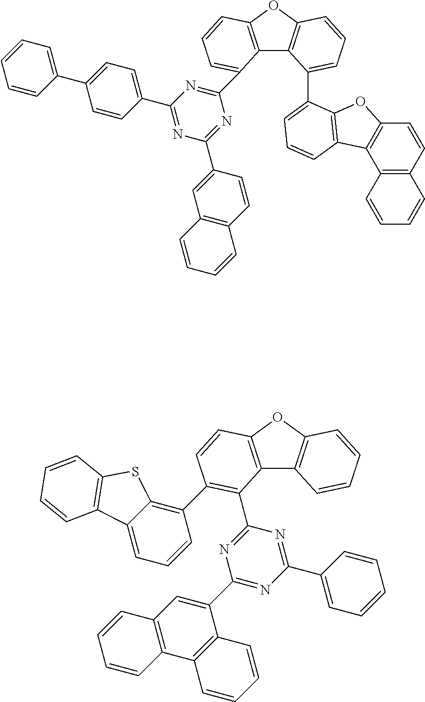 [SMILES: c1ccc(-c2ccc(-c3nc(-c4ccc5ccccc5c4)nc(-c4cccc5oc6cccc(-c7cccc8c7oc7ccc9ccccc9c78)c6c45)n3)cc2)cc1 ; c1ccc(-c2nc(-c3cc4ccccc4c4ccccc34)nc(-c3c(-c4cccc5c4sc4ccccc45)ccc4oc5ccccc5c34)n2)cc1]

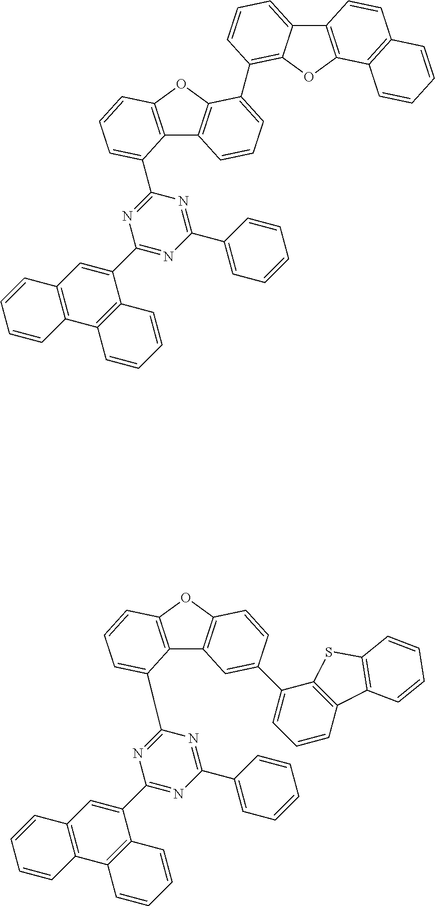 [SMILES: c1ccc(-c2nc(-c3cc4ccccc4c4ccccc34)nc(-c3cccc4oc5c(-c6cccc7c6oc6c8ccccc8ccc76)cccc5c34)n2)cc1 ; c1ccc(-c2nc(-c3cc4ccccc4c4ccccc34)nc(-c3cccc4oc5ccc(-c6cccc7c6sc6ccccc67)cc5c34)n2)cc1]

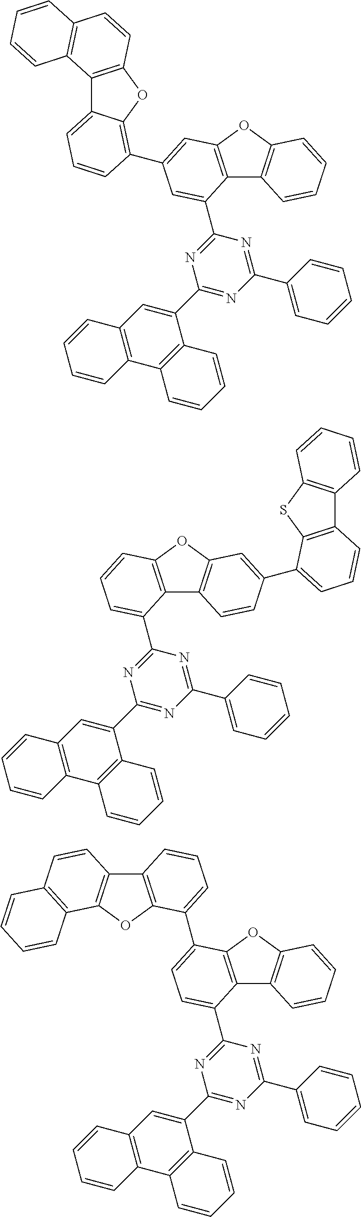

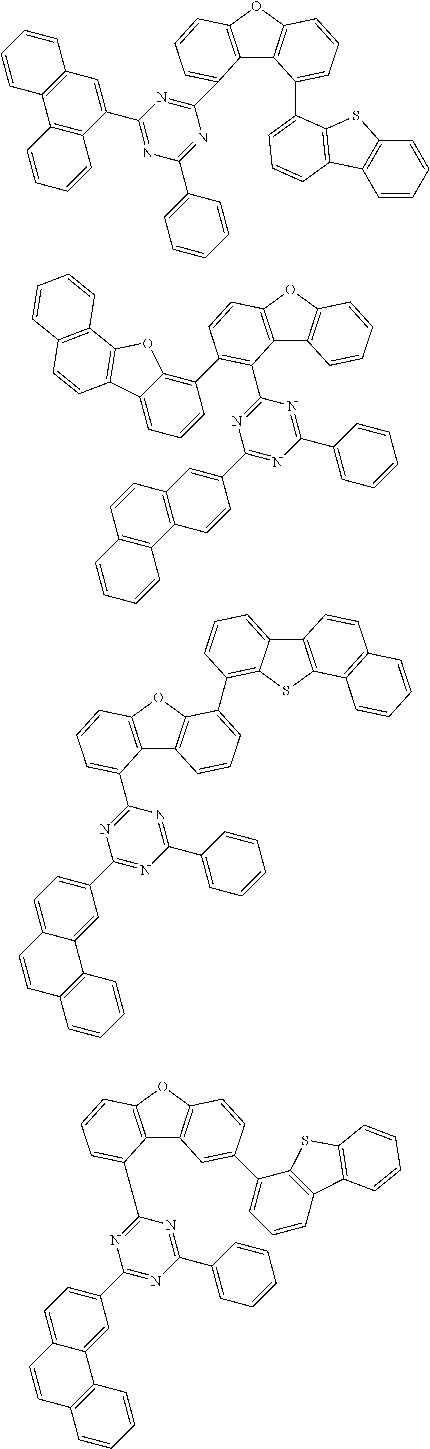 [SMILES: c1ccc(-c2nc(-c3cc4ccccc4c4ccccc34)nc(-c3cccc4oc5cccc(-c6cccc7c6sc6ccccc67)c5c34)n2)cc1 ; c1ccc(-c2nc(-c3ccc4c(ccc5ccccc54)c3)nc(-c3c(-c4cccc5c4oc4c6ccccc6ccc54)ccc4oc5ccccc5c34)n2)cc1 ; c1ccc(-c2nc(-c3ccc4ccc5ccccc5c4c3)nc(-c3cccc4oc5c(-c6cccc7c6sc6c8ccccc8ccc76)cccc5c34)n2)cc1 ; c1ccc(-c2nc(-c3ccc4ccc5ccccc5c4c3)nc(-c3cccc4oc5ccc(-c6cccc7c6sc6ccccc67)cc5c34)n2)cc1]

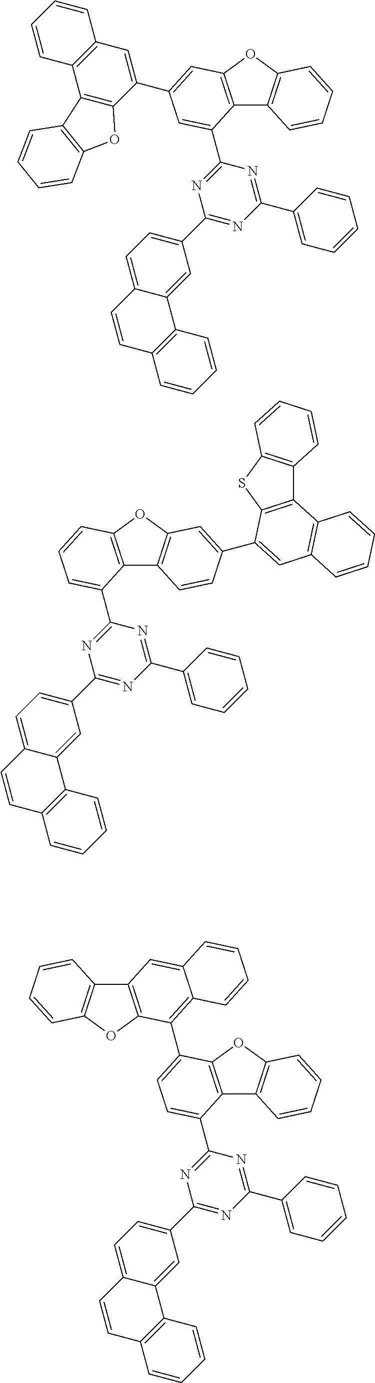 [SMILES: c1ccc(-c2nc(-c3ccc4ccc5ccccc5c4c3)nc(-c3cc(-c4cc5ccccc5c5c4oc4ccccc45)cc4oc5ccccc5c34)n2)cc1 ; c1ccc(-c2nc(-c3ccc4ccc5ccccc5c4c3)nc(-c3cccc4oc5cc(-c6cc7ccccc7c7c6sc6ccccc67)ccc5c34)n2)cc1 ; c1ccc(-c2nc(-c3ccc4ccc5ccccc5c4c3)nc(-c3ccc(-c4c5ccccc5cc5c4oc4ccccc45)c4oc5ccccc5c34)n2)cc1]

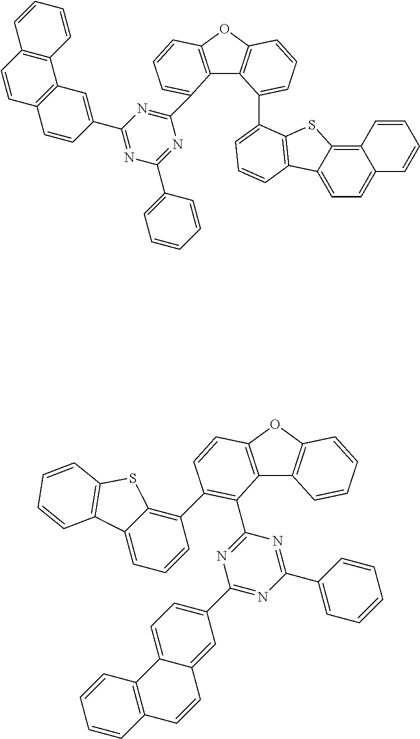 [SMILES: c1ccc(-c2nc(-c3ccc4ccc5ccccc5c4c3)nc(-c3cccc4oc5cccc(-c6cccc7c6sc6c8ccccc8ccc76)c5c34)n2)cc1 ; c1ccc(-c2nc(-c3ccc4c(ccc5ccccc54)c3)nc(-c3c(-c4cccc5c4sc4ccccc45)ccc4oc5ccccc5c34)n2)cc1]

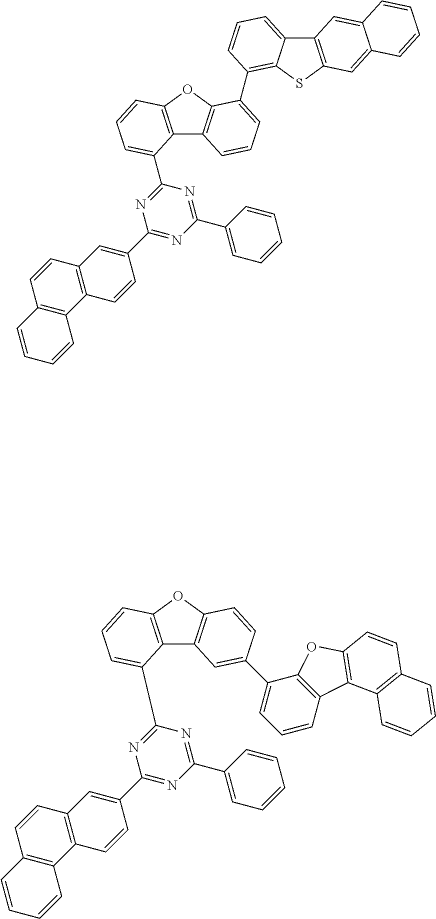 [SMILES: c1ccc(-c2nc(-c3ccc4c(ccc5ccccc54)c3)nc(-c3cccc4oc5c(-c6cccc7c6sc6cc8ccccc8cc67)cccc5c34)n2)cc1 ; c1ccc(-c2nc(-c3ccc4c(ccc5ccccc54)c3)nc(-c3cccc4oc5ccc(-c6cccc7c6oc6ccc8ccccc8c67)cc5c34)n2)cc1]

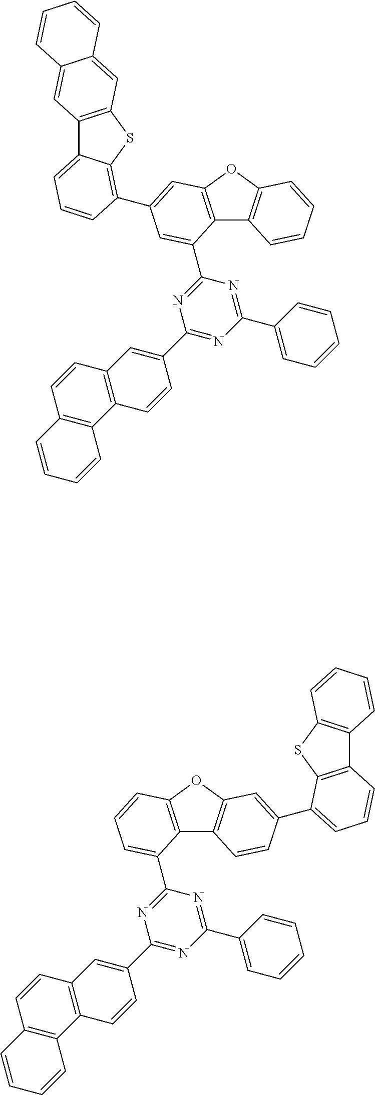 [SMILES: c1ccc(-c2nc(-c3ccc4c(ccc5ccccc54)c3)nc(-c3cc(-c4cccc5c4sc4cc6ccccc6cc45)cc4oc5ccccc5c34)n2)cc1 ; c1ccc(-c2nc(-c3ccc4c(ccc5ccccc54)c3)nc(-c3cccc4oc5cc(-c6cccc7c6sc6ccccc67)ccc5c34)n2)cc1]

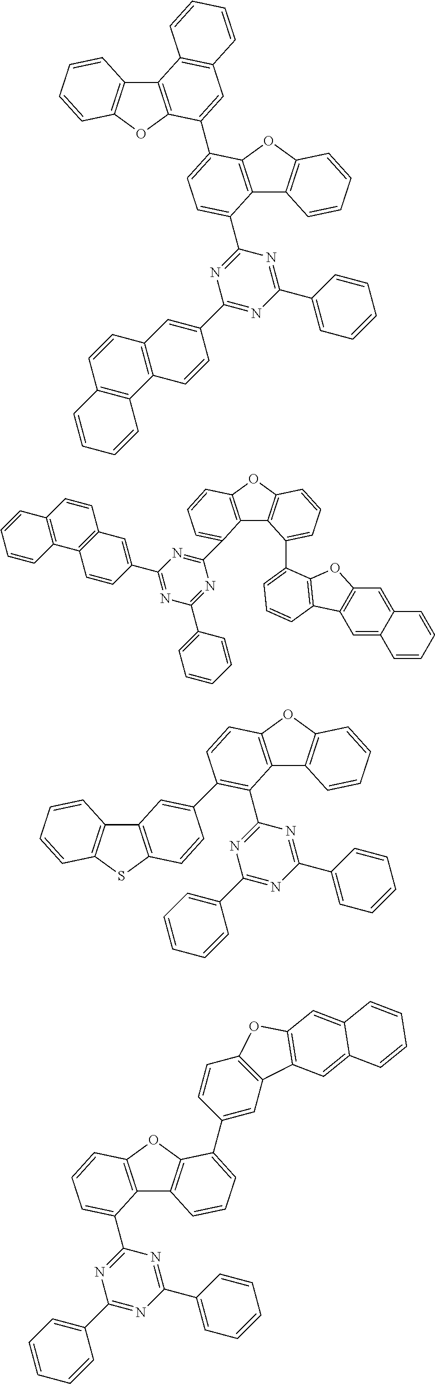

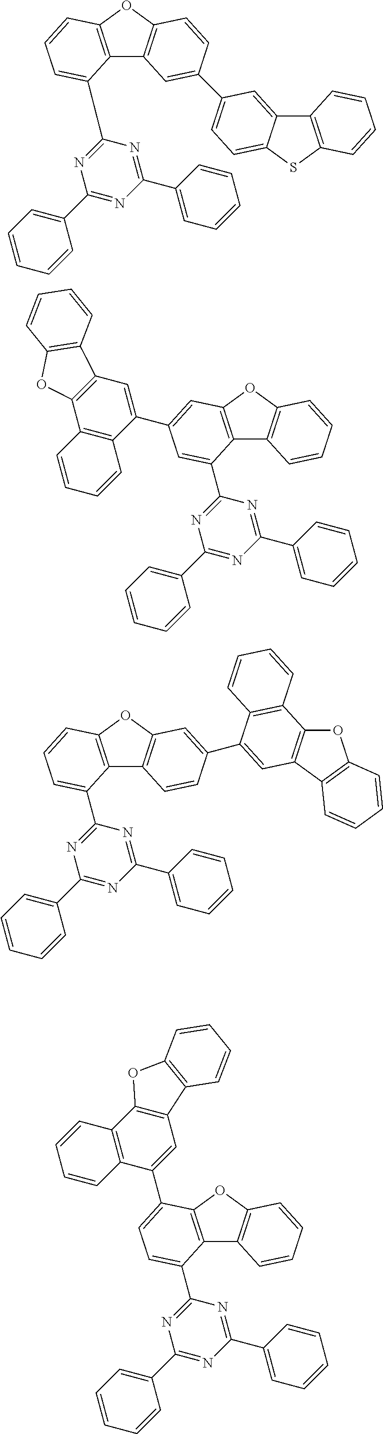

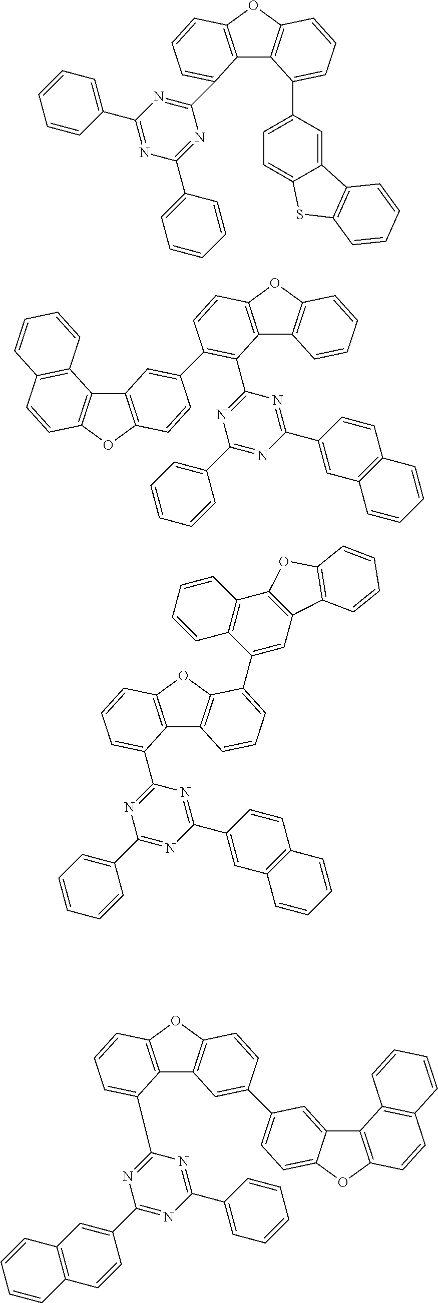

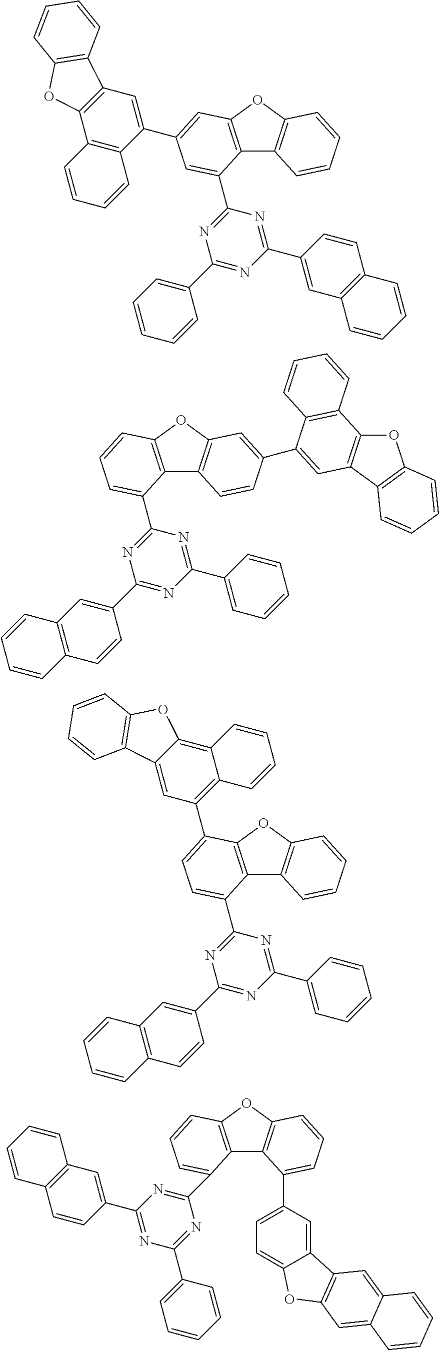

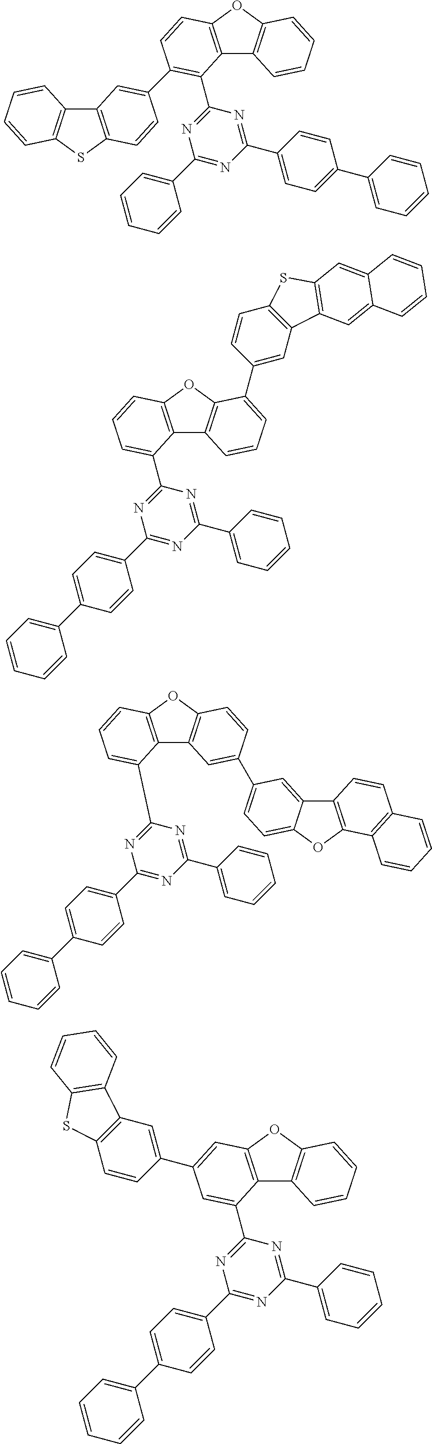

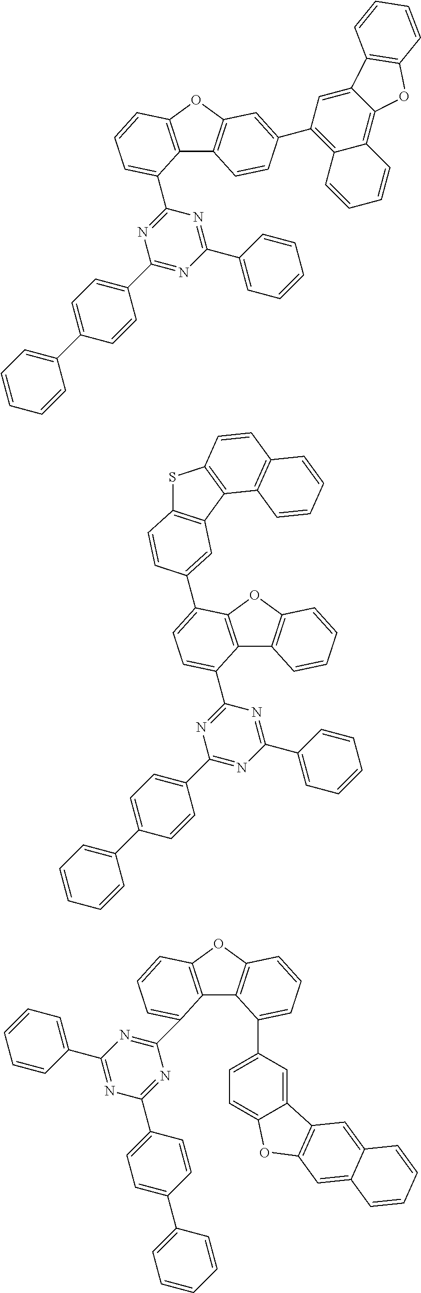 [SMILES: c1ccc(-c2ccc(-c3nc(-c4ccccc4)nc(-c4cccc5oc6cc(-c7cc8c9ccccc9oc8c8ccccc78)ccc6c45)n3)cc2)cc1 ; c1ccc(-c2ccc(-c3nc(-c4ccccc4)nc(-c4ccc(-c5ccc6sc7ccc8ccccc8c7c6c5)c5oc6ccccc6c45)n3)cc2)cc1 ; c1ccc(-c2ccc(-c3nc(-c4ccccc4)nc(-c4cccc5oc6cccc(-c7ccc8oc9cc%10ccccc%10cc9c8c7)c6c45)n3)cc2)cc1]

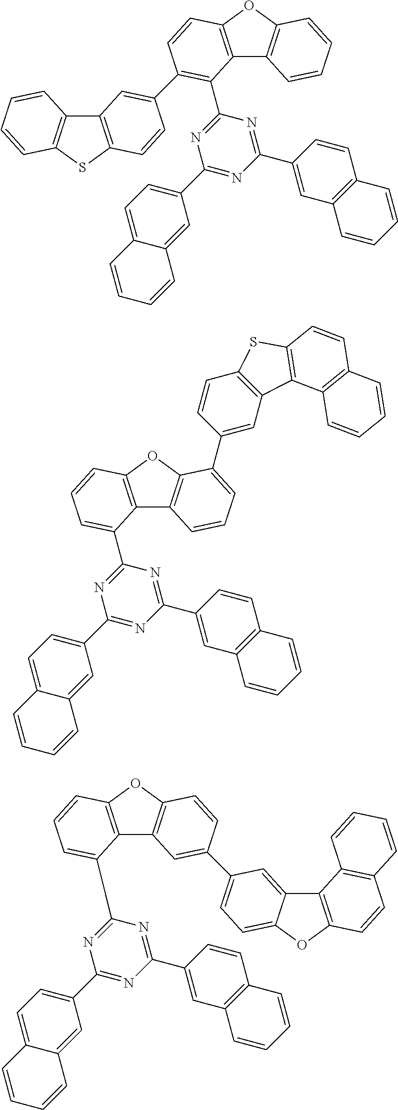

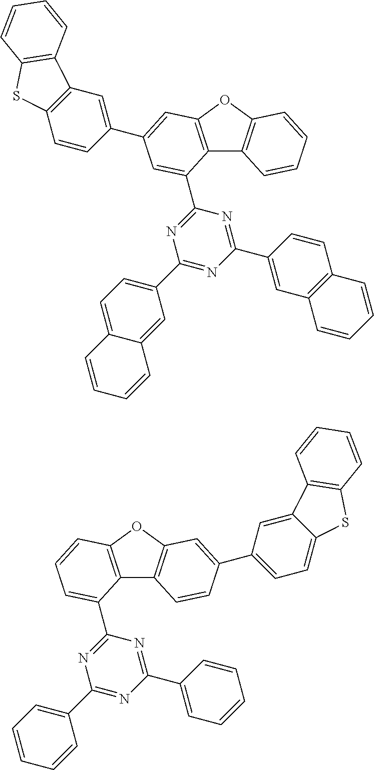 [SMILES: c1ccc2cc(-c3nc(-c4ccc5ccccc5c4)nc(-c4cc(-c5ccc6sc7ccccc7c6c5)cc5oc6ccccc6c45)n3)ccc2c1 ; c1ccc(-c2nc(-c3ccccc3)nc(-c3cccc4oc5cc(-c6ccc7sc8ccccc8c7c6)ccc5c34)n2)cc1]

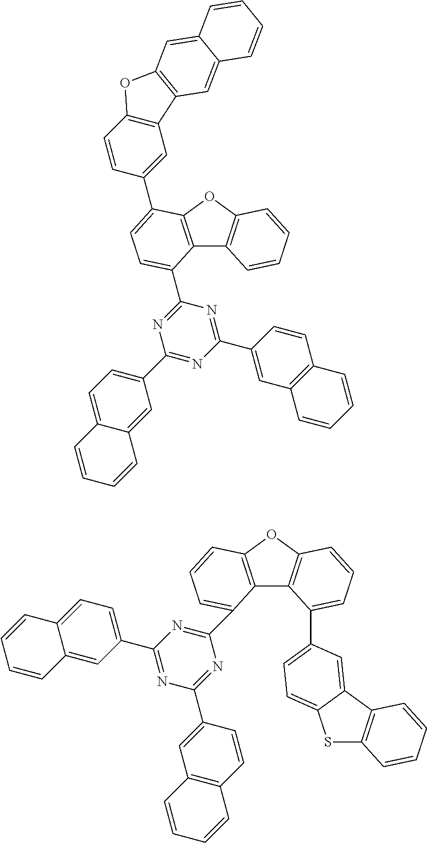 [SMILES: c1ccc2cc(-c3nc(-c4ccc5ccccc5c4)nc(-c4ccc(-c5ccc6oc7cc8ccccc8cc7c6c5)c5oc6ccccc6c45)n3)ccc2c1 ; c1ccc2cc(-c3nc(-c4ccc5ccccc5c4)nc(-c4cccc5oc6cccc(-c7ccc8sc9ccccc9c8c7)c6c45)n3)ccc2c1]

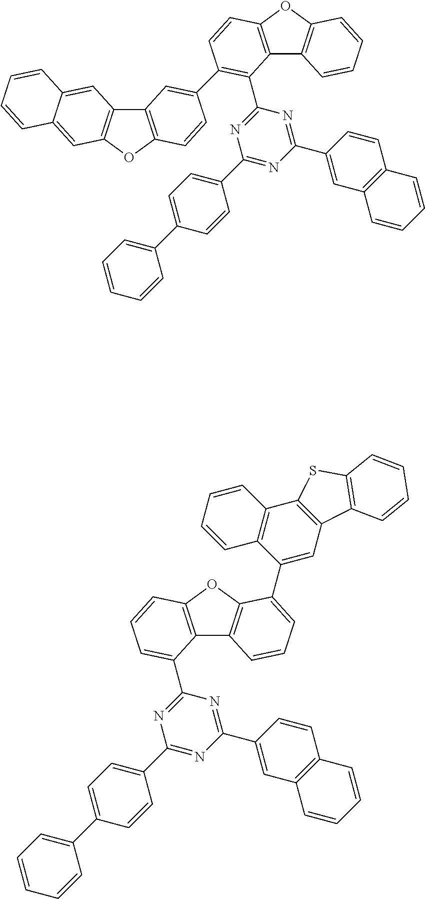 [SMILES: c1ccc(-c2ccc(-c3nc(-c4ccc5ccccc5c4)nc(-c4c(-c5ccc6oc7cc8ccccc8cc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8c9ccccc9sc8c8ccccc78)cccc6c45)n3)cc2)cc1]

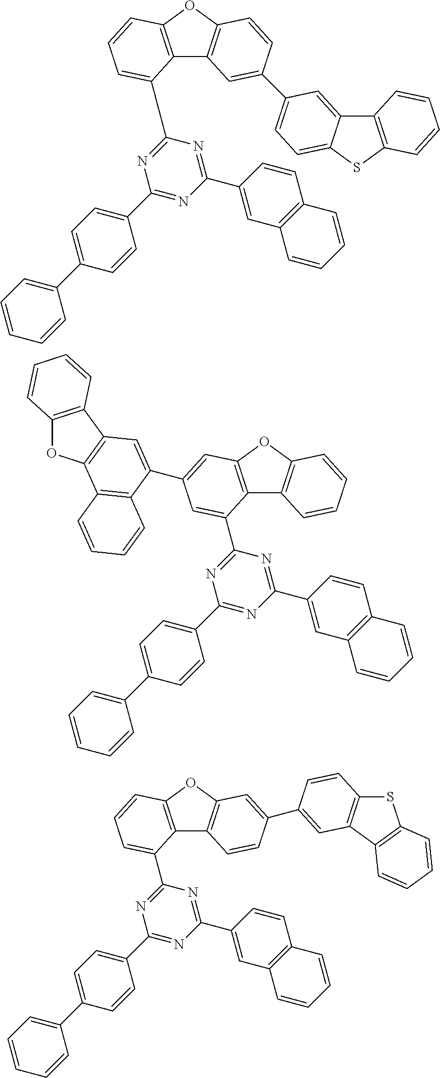

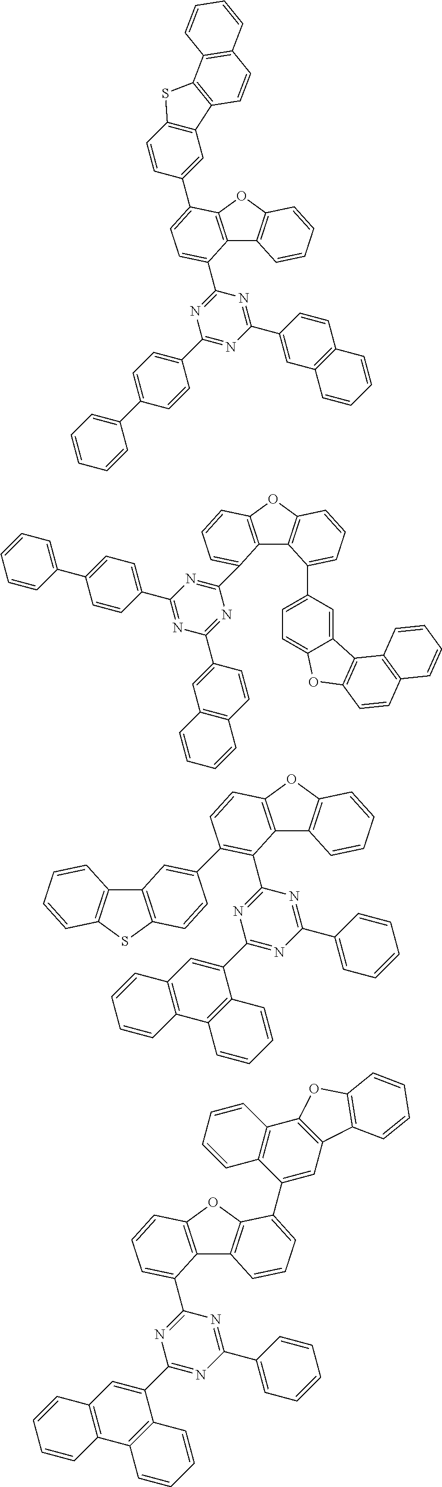

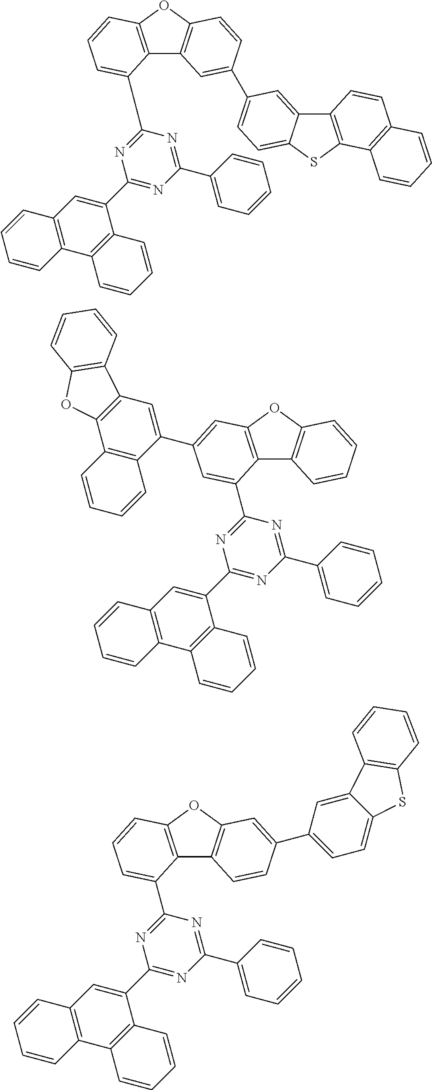

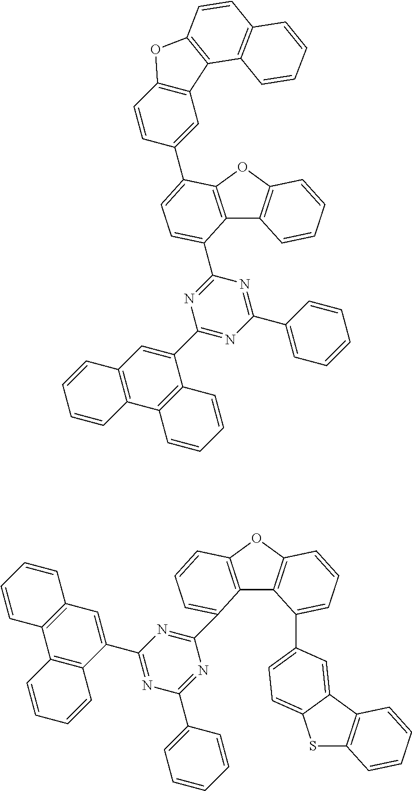 [SMILES: c1ccc(-c2nc(-c3cc4ccccc4c4ccccc34)nc(-c3ccc(-c4ccc5oc6ccc7ccccc7c6c5c4)c4oc5ccccc5c34)n2)cc1 ; c1ccc(-c2nc(-c3cc4ccccc4c4ccccc34)nc(-c3cccc4oc5cccc(-c6ccc7sc8ccccc8c7c6)c5c34)n2)cc1]

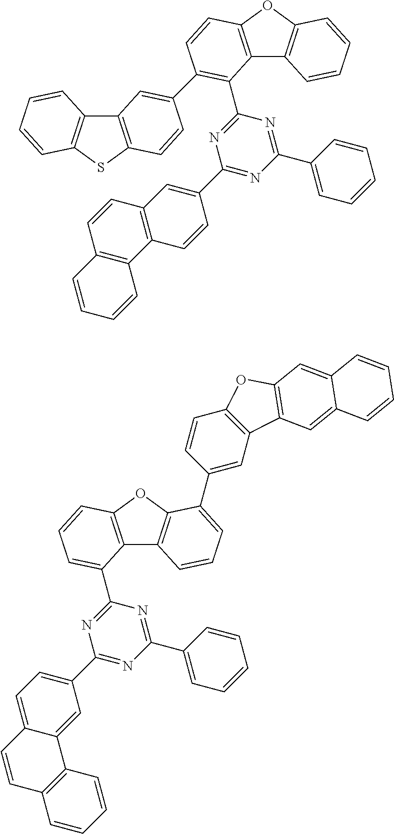 [SMILES: c1ccc(-c2nc(-c3ccc4c(ccc5ccccc54)c3)nc(-c3c(-c4ccc5sc6ccccc6c5c4)ccc4oc5ccccc5c34)n2)cc1 ; c1ccc(-c2nc(-c3ccc4ccc5ccccc5c4c3)nc(-c3cccc4oc5c(-c6ccc7oc8cc9ccccc9cc8c7c6)cccc5c34)n2)cc1]

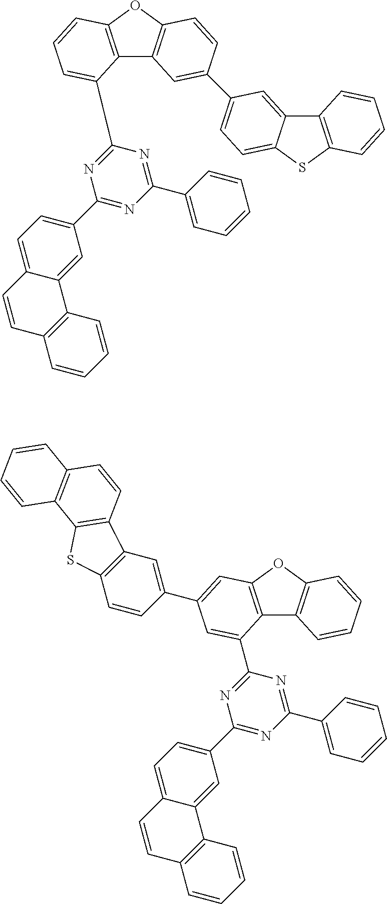 [SMILES: c1ccc(-c2nc(-c3ccc4ccc5ccccc5c4c3)nc(-c3cccc4oc5ccc(-c6ccc7sc8ccccc8c7c6)cc5c34)n2)cc1 ; c1ccc(-c2nc(-c3ccc4ccc5ccccc5c4c3)nc(-c3cc(-c4ccc5sc6c7ccccc7ccc6c5c4)cc4oc5ccccc5c34)n2)cc1]

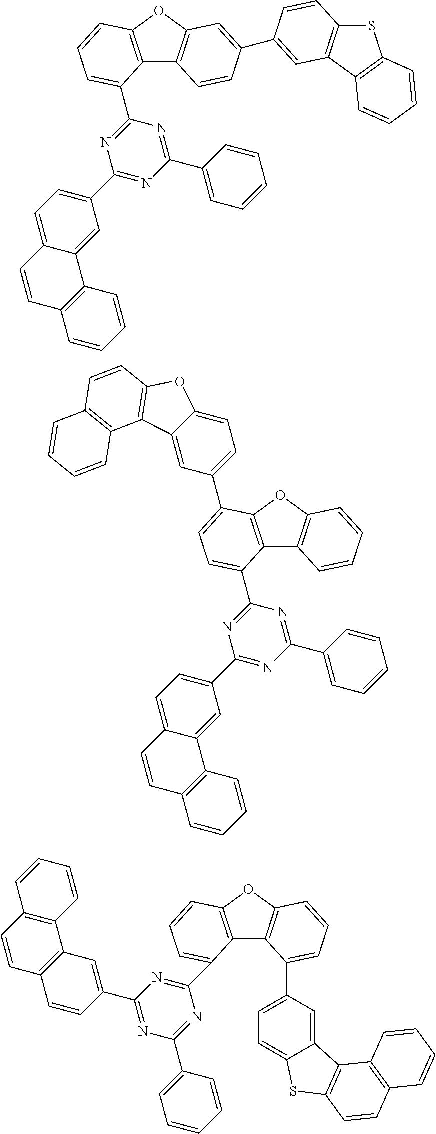

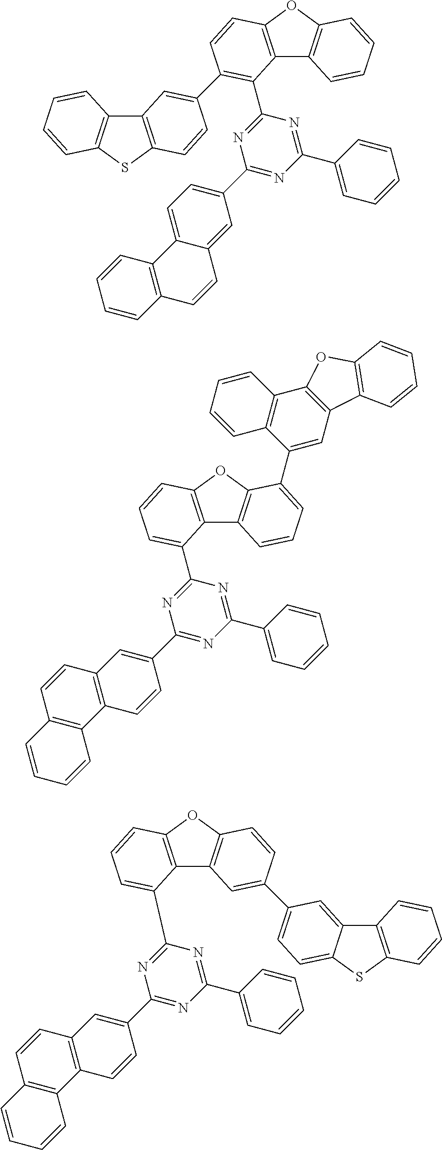 [SMILES: c1ccc(-c2nc(-c3ccc4c(ccc5ccccc54)c3)nc(-c3c(-c4ccc5sc6ccccc6c5c4)ccc4oc5ccccc5c34)n2)cc1 ; c1ccc(-c2nc(-c3ccc4c(ccc5ccccc54)c3)nc(-c3cccc4oc5c(-c6cc7c8ccccc8oc7c7ccccc67)cccc5c34)n2)cc1 ; c1ccc(-c2nc(-c3ccc4c(ccc5ccccc54)c3)nc(-c3cccc4oc5ccc(-c6ccc7sc8ccccc8c7c6)cc5c34)n2)cc1]

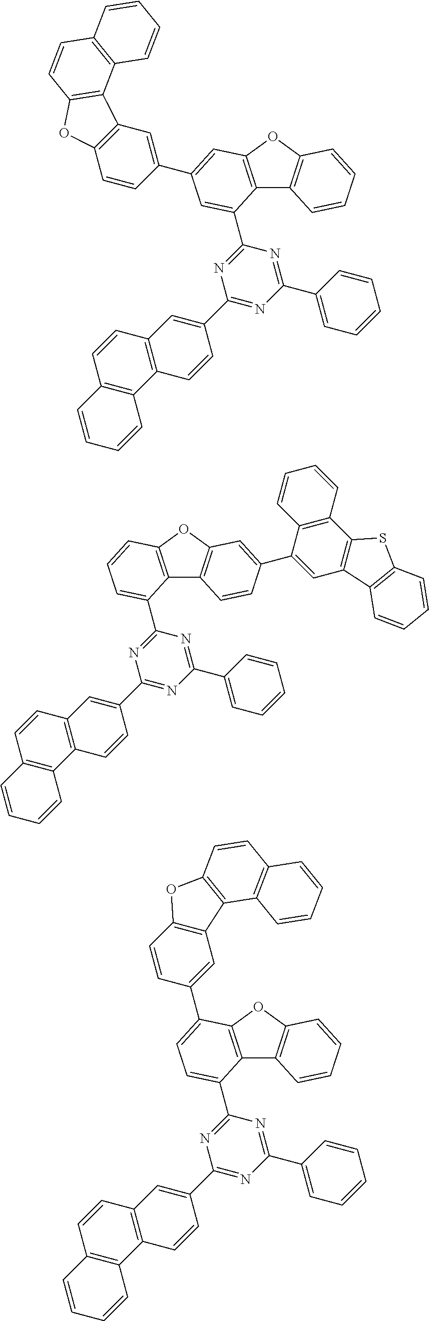 [SMILES: c1ccc(-c2nc(-c3ccc4c(ccc5ccccc54)c3)nc(-c3cc(-c4ccc5oc6ccc7ccccc7c6c5c4)cc4oc5ccccc5c34)n2)cc1 ; c1ccc(-c2nc(-c3ccc4c(ccc5ccccc54)c3)nc(-c3cccc4oc5cc(-c6cc7c8ccccc8sc7c7ccccc67)ccc5c34)n2)cc1 ; c1ccc(-c2nc(-c3ccc4c(ccc5ccccc54)c3)nc(-c3ccc(-c4ccc5oc6ccc7ccccc7c6c5c4)c4oc5ccccc5c34)n2)cc1]

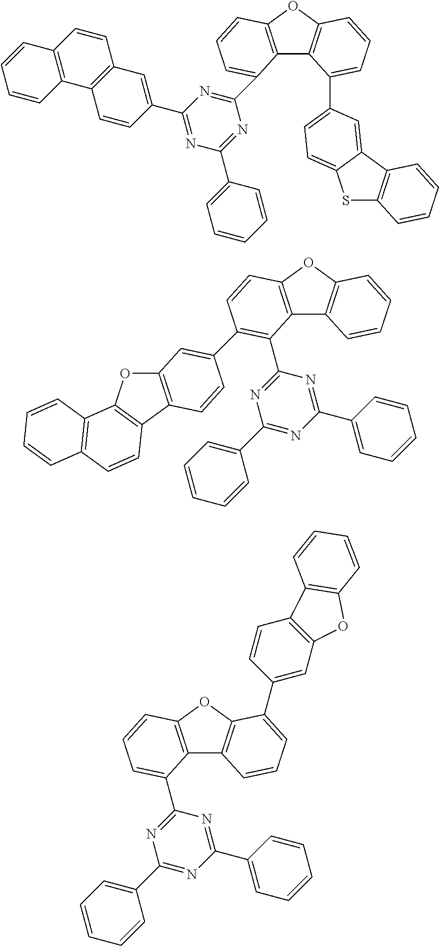

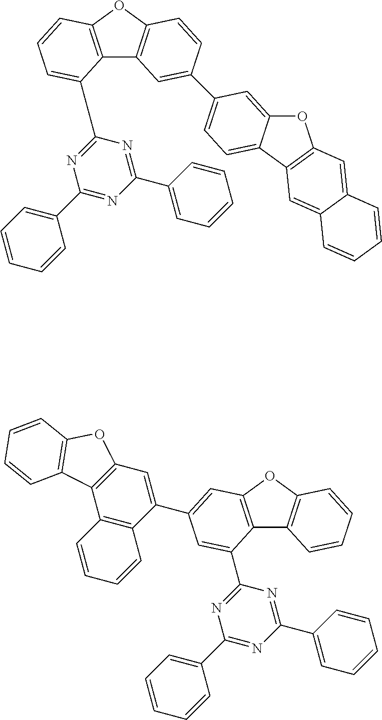 [SMILES: c1ccc(-c2nc(-c3ccccc3)nc(-c3cccc4oc5ccc(-c6ccc7c(c6)oc6cc8ccccc8cc67)cc5c34)n2)cc1 ; c1ccc(-c2nc(-c3ccccc3)nc(-c3cc(-c4cc5oc6ccccc6c5c5ccccc45)cc4oc5ccccc5c34)n2)cc1]

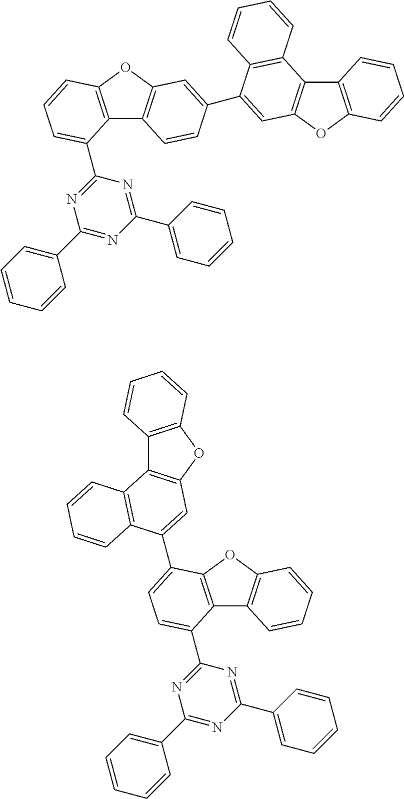 [SMILES: c1ccc(-c2nc(-c3ccccc3)nc(-c3cccc4oc5cc(-c6cc7oc8ccccc8c7c7ccccc67)ccc5c34)n2)cc1 ; c1ccc(-c2nc(-c3ccccc3)nc(-c3ccc(-c4cc5oc6ccccc6c5c5ccccc45)c4oc5ccccc5c34)n2)cc1]

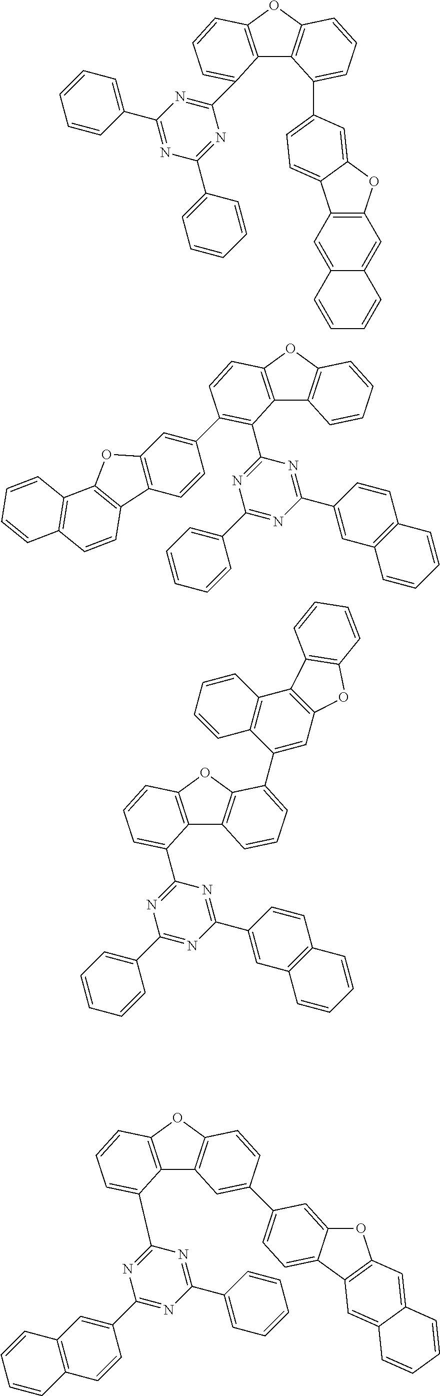

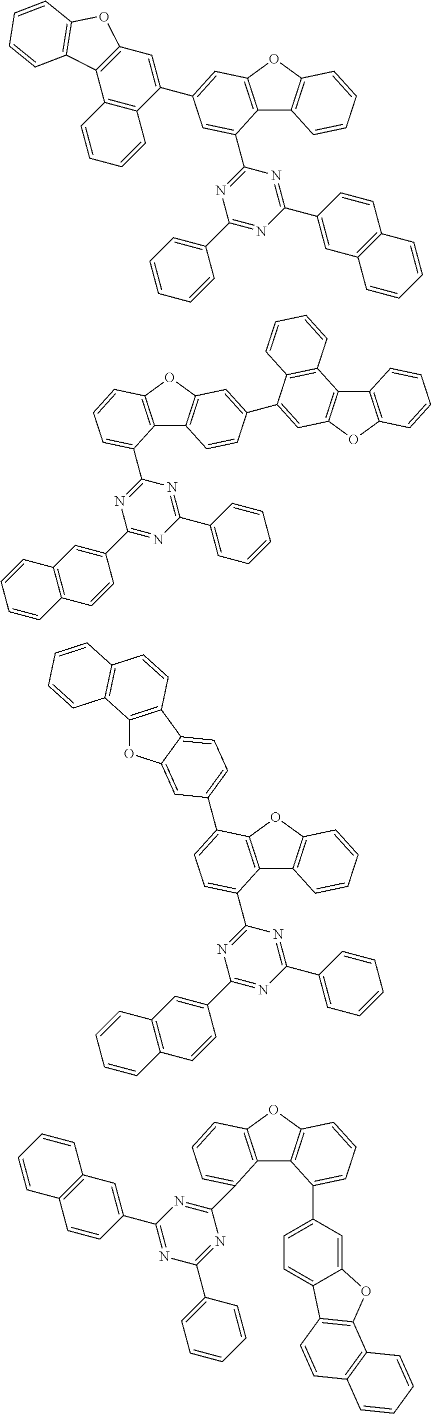

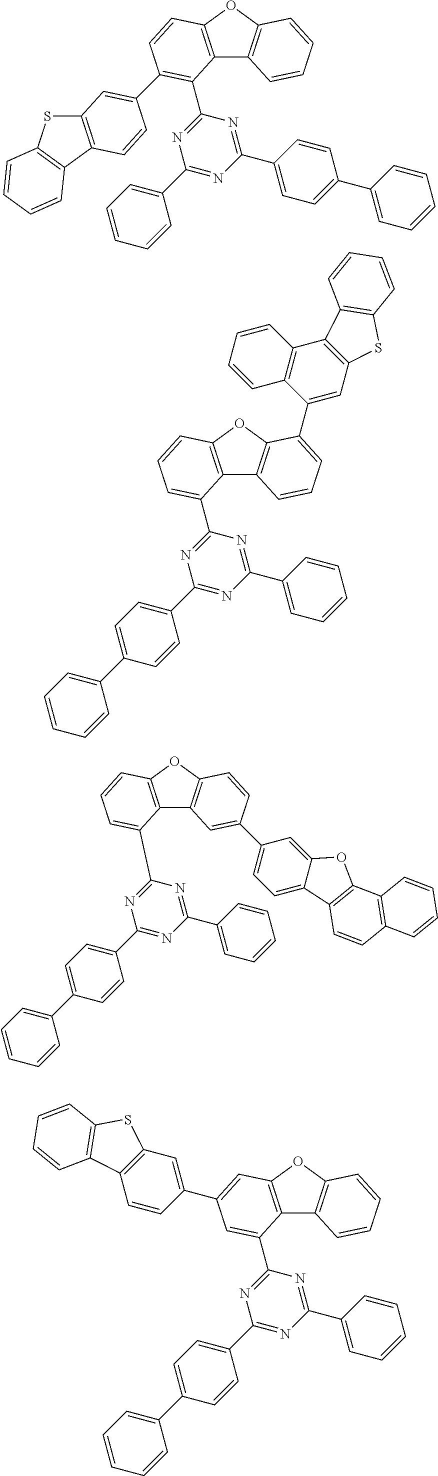

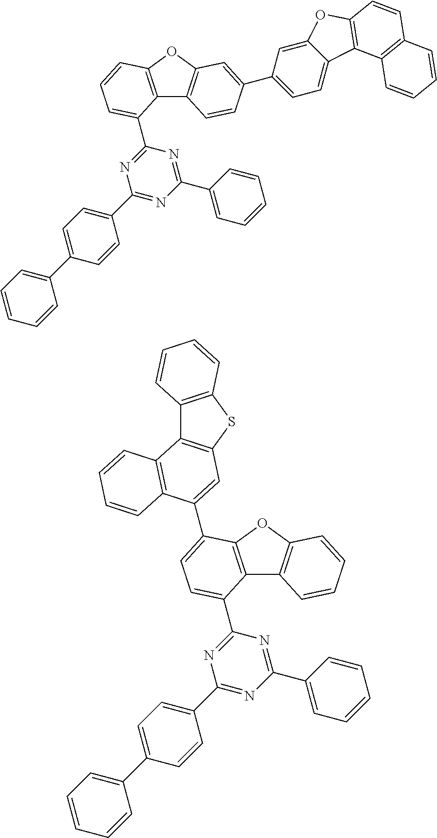 [SMILES: c1ccc(-c2ccc(-c3nc(-c4ccccc4)nc(-c4cccc5oc6cc(-c7ccc8c(c7)oc7ccc9ccccc9c78)ccc6c45)n3)cc2)cc1 ; c1ccc(-c2ccc(-c3nc(-c4ccccc4)nc(-c4ccc(-c5cc6sc7ccccc7c6c6ccccc56)c5oc6ccccc6c45)n3)cc2)cc1]

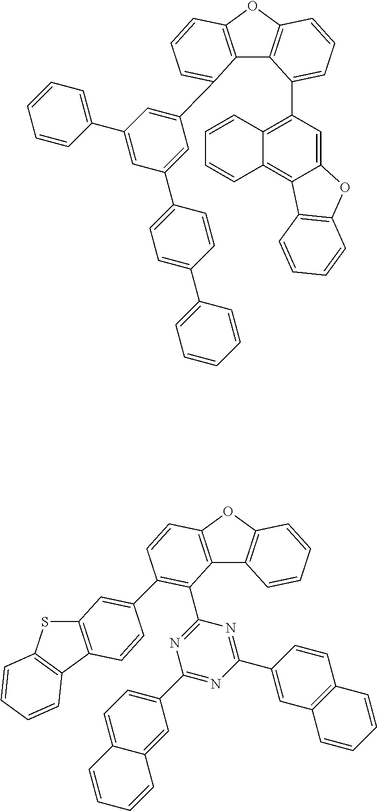 [SMILES: c1ccc(-c2ccc(-c3cc(-c4ccccc4)cc(-c4cccc5oc6cccc(-c7cc8oc9ccccc9c8c8ccccc78)c6c45)c3)cc2)cc1 ; c1ccc2cc(-c3nc(-c4ccc5ccccc5c4)nc(-c4c(-c5ccc6c(c5)sc5ccccc56)ccc5oc6ccccc6c45)n3)ccc2c1]

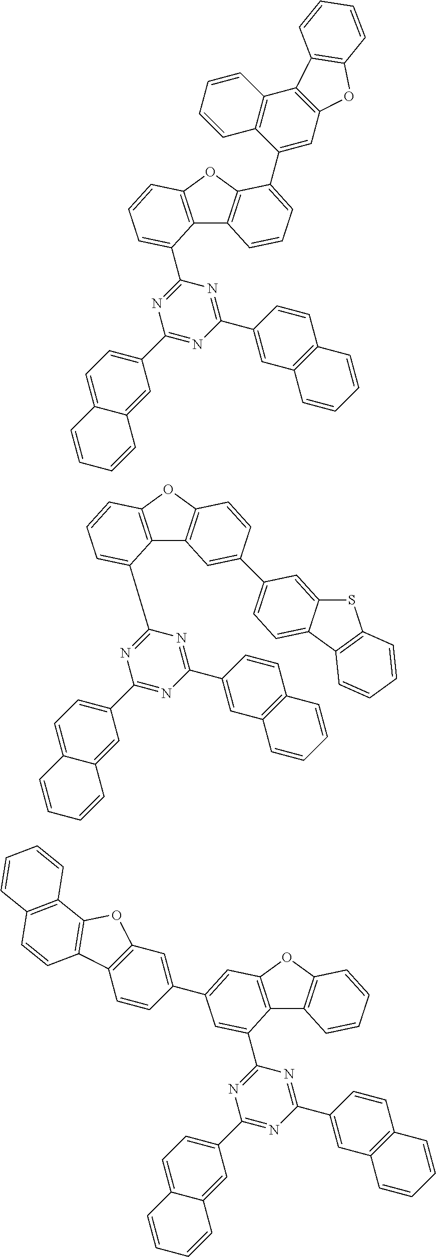

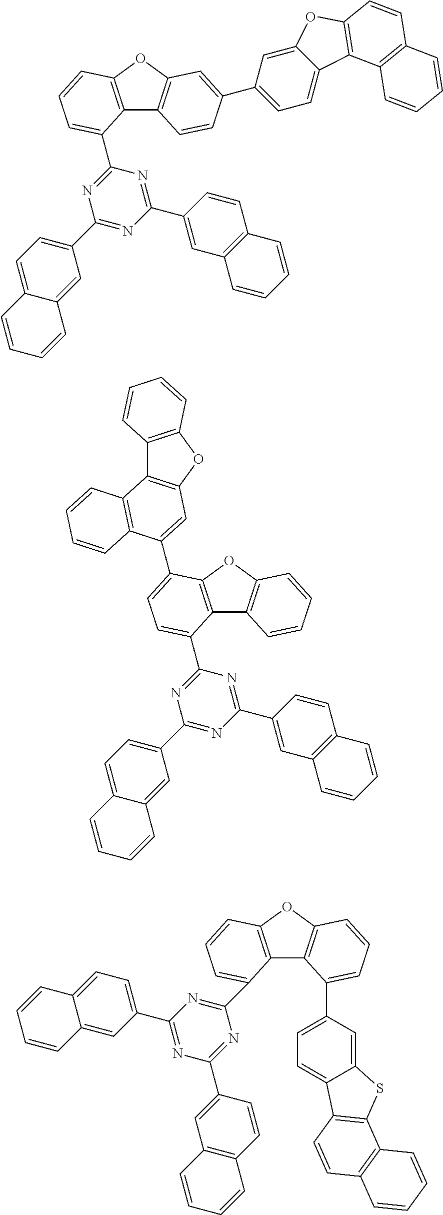 [SMILES: c1ccc2cc(-c3nc(-c4ccc5ccccc5c4)nc(-c4cccc5oc6cc(-c7ccc8c(c7)oc7ccc9ccccc9c78)ccc6c45)n3)ccc2c1 ; c1ccc2cc(-c3nc(-c4ccc5ccccc5c4)nc(-c4ccc(-c5cc6oc7ccccc7c6c6ccccc56)c5oc6ccccc6c45)n3)ccc2c1 ; c1ccc2cc(-c3nc(-c4ccc5ccccc5c4)nc(-c4cccc5oc6cccc(-c7ccc8c(c7)sc7c9ccccc9ccc87)c6c45)n3)ccc2c1]

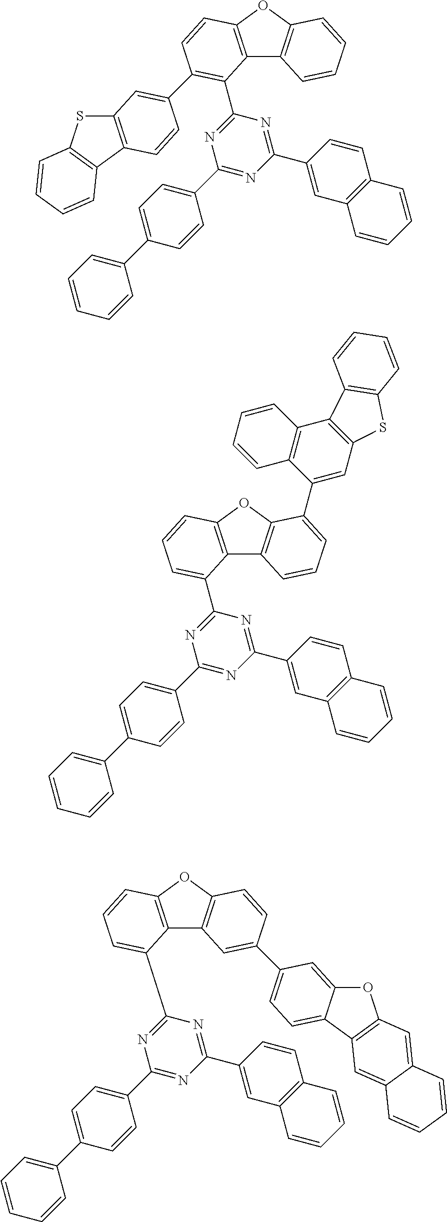 [SMILES: c1ccc(-c2ccc(-c3nc(-c4ccc5ccccc5c4)nc(-c4c(-c5ccc6c(c5)sc5ccccc56)ccc5oc6ccccc6c45)n3)cc2)cc1 ; c1ccc(-c2ccc(-c3nc(-c4ccc5ccccc5c4)nc(-c4cccc5oc6c(-c7cc8sc9ccccc9c8c8ccccc78)cccc6c45)n3)cc2)cc1 ; c1ccc(-c2ccc(-c3nc(-c4ccc5ccccc5c4)nc(-c4cccc5oc6ccc(-c7ccc8c(c7)oc7cc9ccccc9cc78)cc6c45)n3)cc2)cc1]

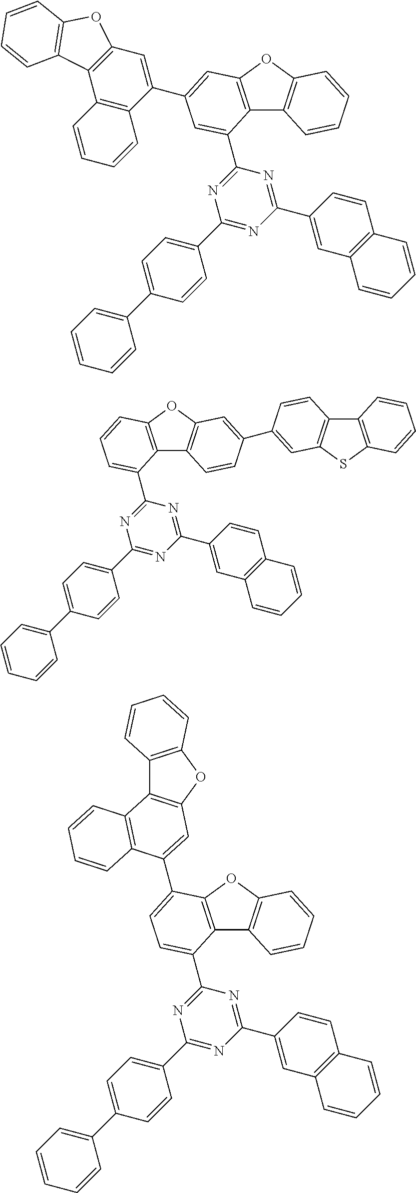

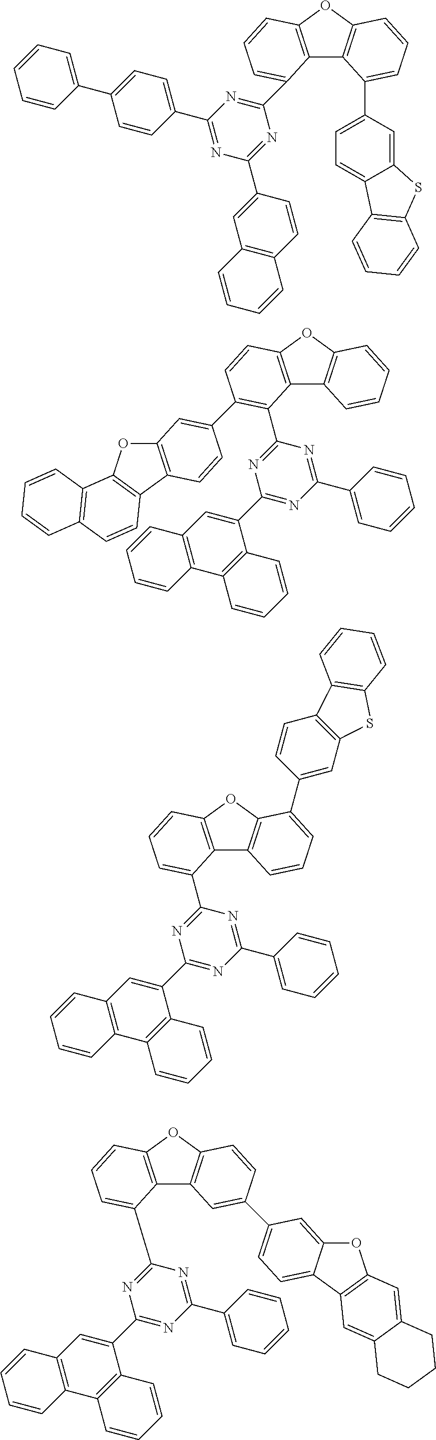

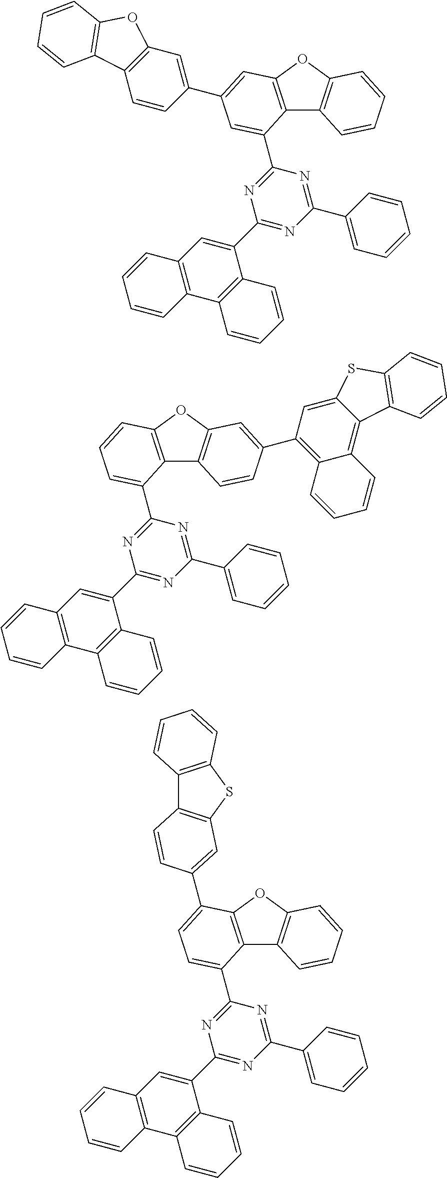

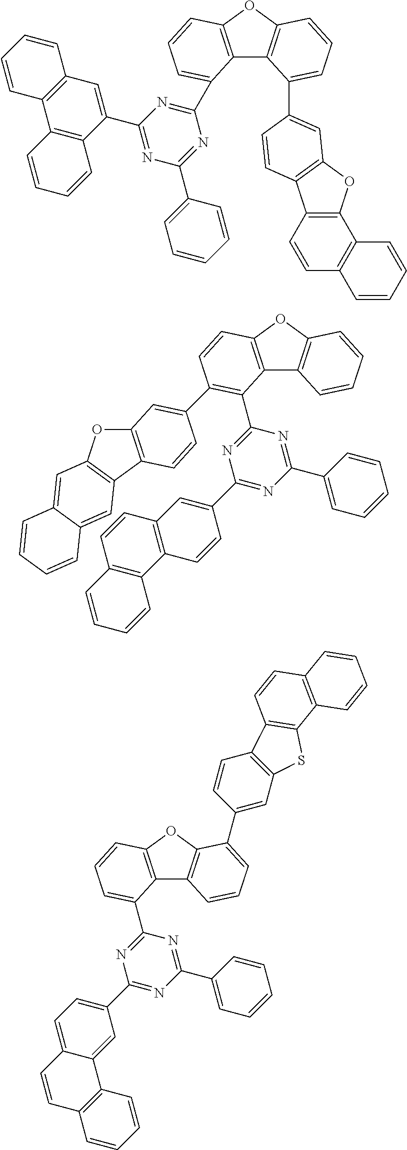

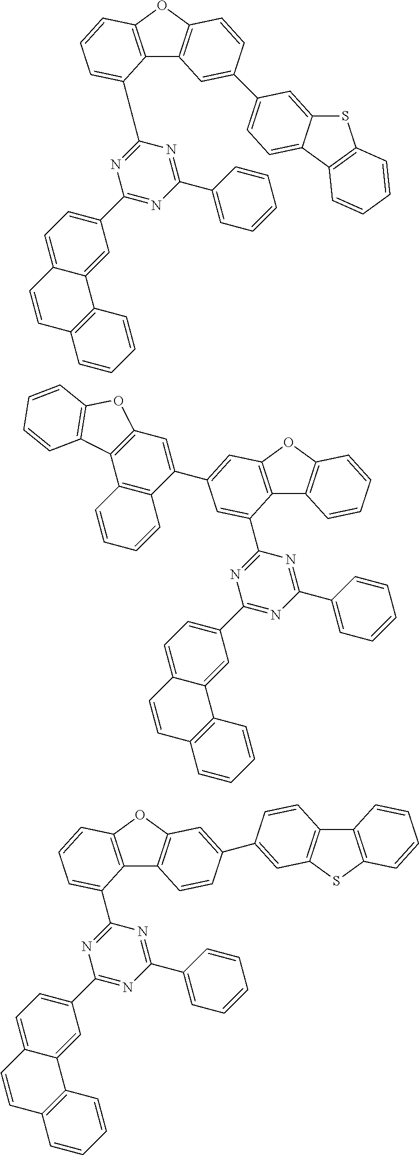

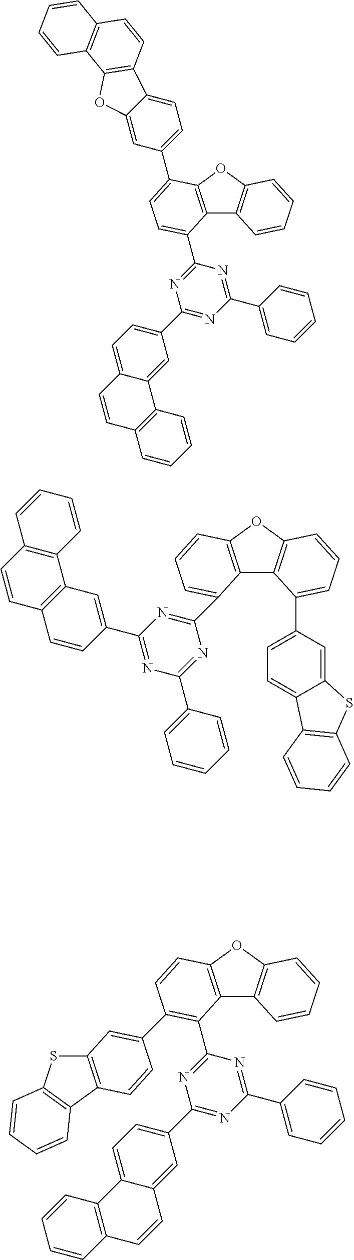 [SMILES: c1ccc(-c2nc(-c3ccc4ccc5ccccc5c4c3)nc(-c3ccc(-c4ccc5c(c4)oc4c6ccccc6ccc54)c4oc5ccccc5c34)n2)cc1 ; c1ccc(-c2nc(-c3ccc4ccc5ccccc5c4c3)nc(-c3cccc4oc5cccc(-c6ccc7c(c6)sc6ccccc67)c5c34)n2)cc1 ; c1ccc(-c2nc(-c3ccc4c(ccc5ccccc54)c3)nc(-c3c(-c4ccc5c(c4)sc4ccccc45)ccc4oc5ccccc5c34)n2)cc1]

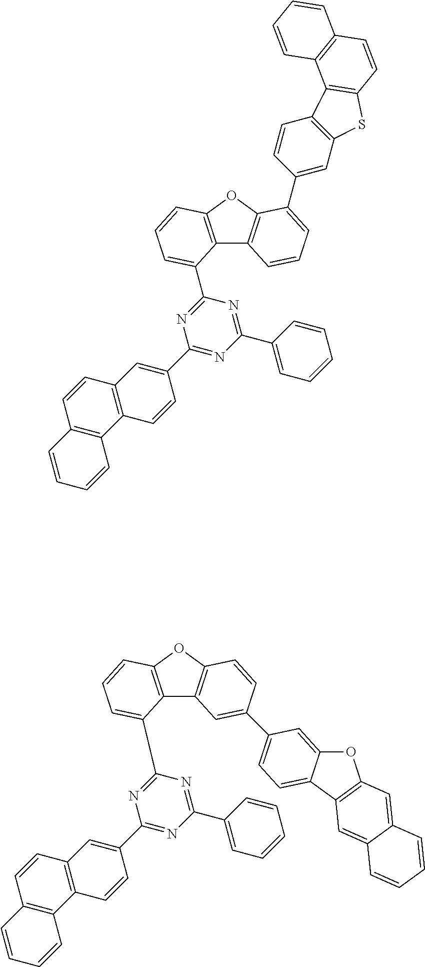 [SMILES: c1ccc(-c2nc(-c3ccc4c(ccc5ccccc54)c3)nc(-c3cccc4oc5c(-c6ccc7c(c6)sc6ccc8ccccc8c67)cccc5c34)n2)cc1 ; c1ccc(-c2nc(-c3ccc4c(ccc5ccccc54)c3)nc(-c3cccc4oc5ccc(-c6ccc7c(c6)oc6cc8ccccc8cc67)cc5c34)n2)cc1]

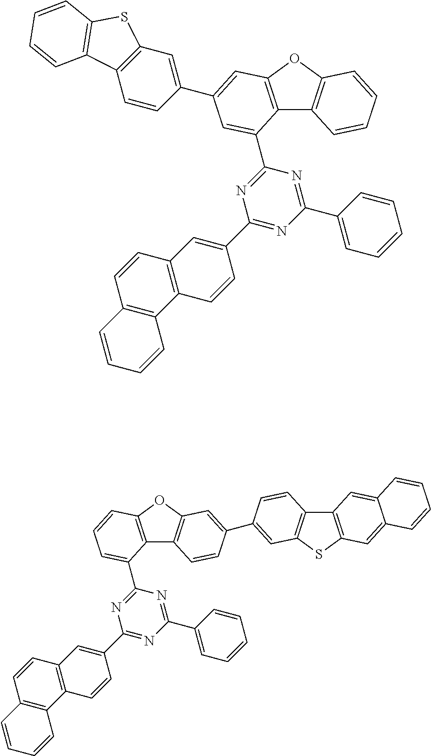 [SMILES: c1ccc(-c2nc(-c3ccc4c(ccc5ccccc54)c3)nc(-c3cc(-c4ccc5c(c4)sc4ccccc45)cc4oc5ccccc5c34)n2)cc1 ; c1ccc(-c2nc(-c3ccc4c(ccc5ccccc54)c3)nc(-c3cccc4oc5cc(-c6ccc7c(c6)sc6cc8ccccc8cc67)ccc5c34)n2)cc1]

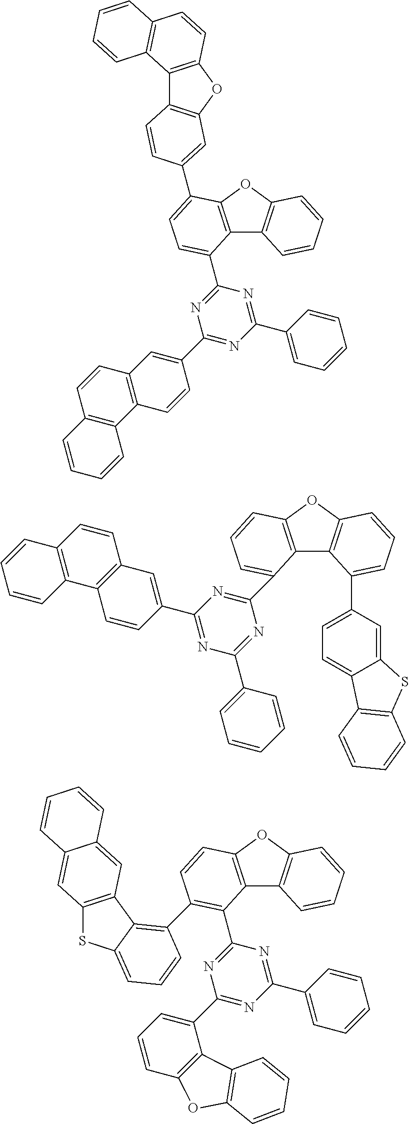 [SMILES: c1ccc(-c2nc(-c3ccc4c(ccc5ccccc54)c3)nc(-c3ccc(-c4ccc5c(c4)oc4ccc6ccccc6c45)c4oc5ccccc5c34)n2)cc1 ; c1ccc(-c2nc(-c3ccc4c(ccc5ccccc54)c3)nc(-c3cccc4oc5cccc(-c6ccc7c(c6)sc6ccccc67)c5c34)n2)cc1 ; c1ccc(-c2nc(-c3cccc4oc5ccccc5c34)nc(-c3c(-c4cccc5sc6cc7ccccc7cc6c45)ccc4oc5ccccc5c34)n2)cc1]

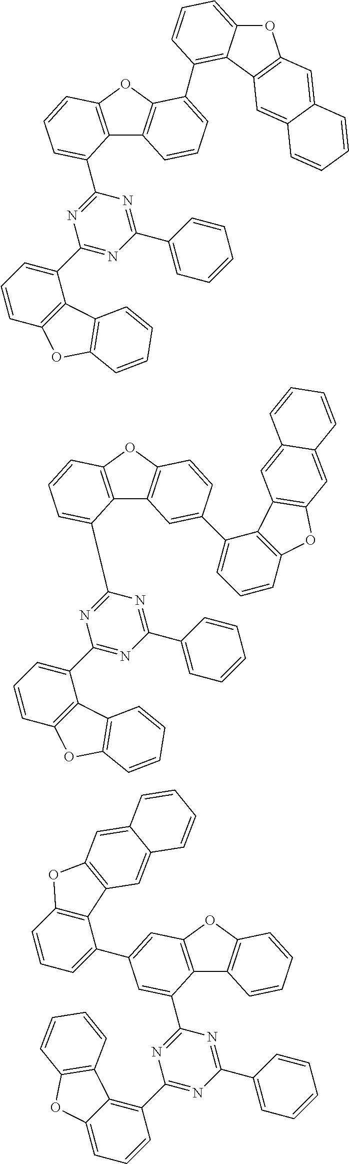

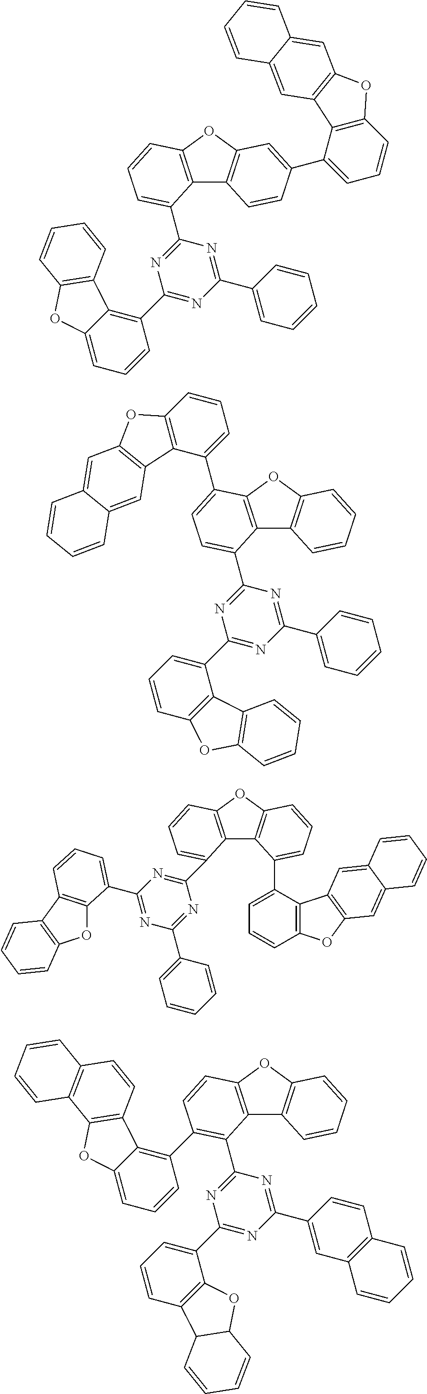

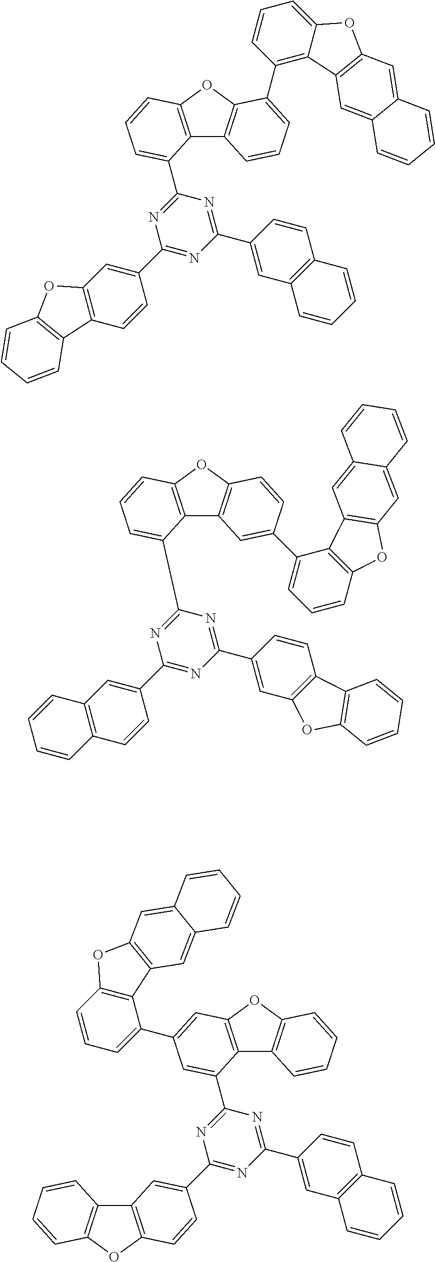 [SMILES: c1ccc2cc(-c3nc(-c4ccc5c(c4)oc4ccccc45)nc(-c4cccc5oc6c(-c7cccc8oc9cc%10ccccc%10cc9c78)cccc6c45)n3)ccc2c1 ; c1ccc2cc(-c3nc(-c4ccc5c(c4)oc4ccccc45)nc(-c4cccc5oc6ccc(-c7cccc8oc9cc%10ccccc%10cc9c78)cc6c45)n3)ccc2c1 ; c1ccc2cc(-c3nc(-c4ccc5oc6ccccc6c5c4)nc(-c4cc(-c5cccc6oc7cc8ccccc8cc7c56)cc5oc6ccccc6c45)n3)ccc2c1]

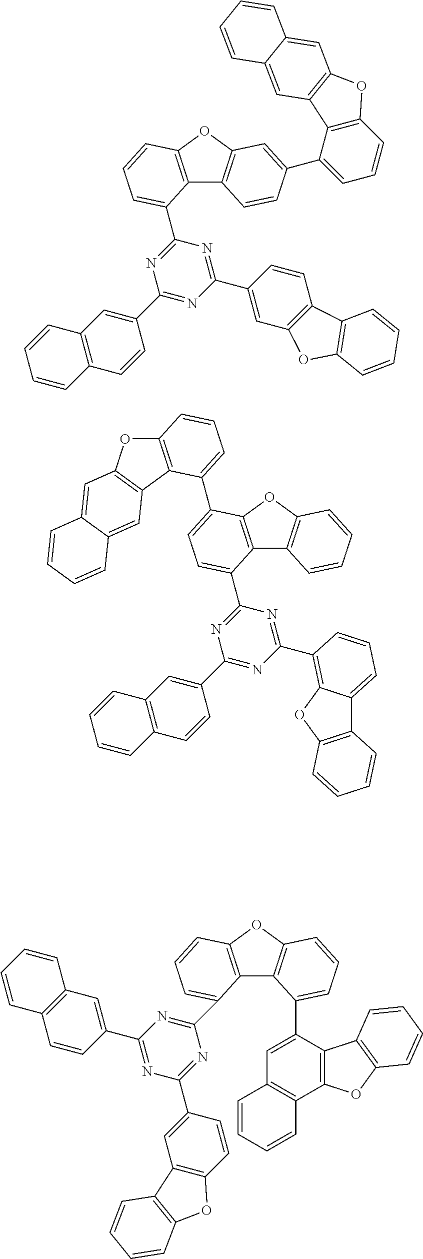 [SMILES: c1ccc2cc(-c3nc(-c4ccc5c(c4)oc4ccccc45)nc(-c4cccc5oc6cc(-c7cccc8oc9cc%10ccccc%10cc9c78)ccc6c45)n3)ccc2c1 ; c1ccc2cc(-c3nc(-c4cccc5c4oc4ccccc45)nc(-c4ccc(-c5cccc6oc7cc8ccccc8cc7c56)c5oc6ccccc6c45)n3)ccc2c1 ; c1ccc2cc(-c3nc(-c4ccc5oc6ccccc6c5c4)nc(-c4cccc5oc6cccc(-c7cc8ccccc8c8oc9ccccc9c78)c6c45)n3)ccc2c1]

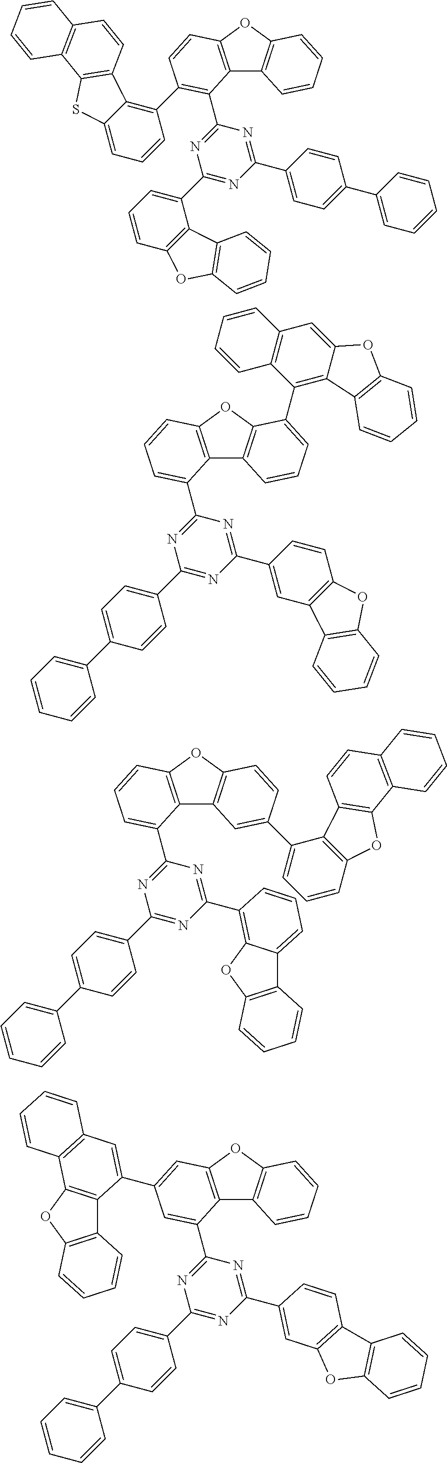

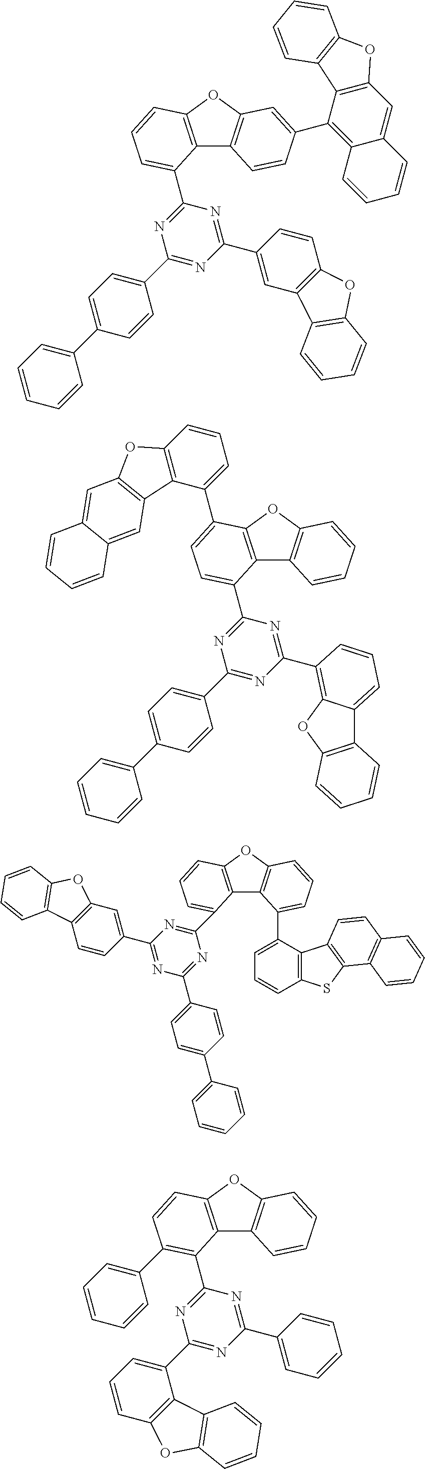 [SMILES: c1ccc(-c2ccc(-c3nc(-c4ccc5oc6ccccc6c5c4)nc(-c4cccc5oc6cc(-c7c8ccccc8cc8oc9ccccc9c78)ccc6c45)n3)cc2)cc1 ; c1ccc(-c2ccc(-c3nc(-c4cccc5c4oc4ccccc45)nc(-c4ccc(-c5cccc6oc7cc8ccccc8cc7c56)c5oc6ccccc6c45)n3)cc2)cc1 ; c1ccc(-c2ccc(-c3nc(-c4ccc5c(c4)oc4ccccc45)nc(-c4cccc5oc6cccc(-c7cccc8sc9c%10ccccc%10ccc9c78)c6c45)n3)cc2)cc1 ; c1ccc(-c2nc(-c3cccc4oc5ccccc5c34)nc(-c3c(-c4ccccc4)ccc4oc5ccccc5c34)n2)cc1]

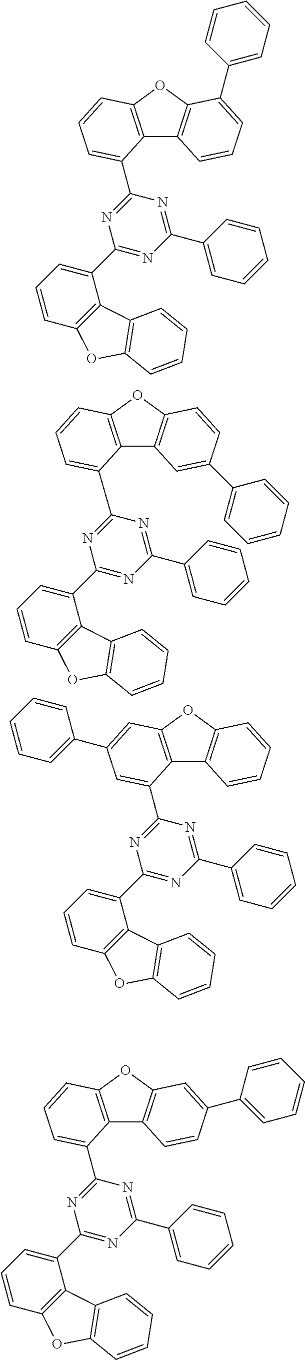

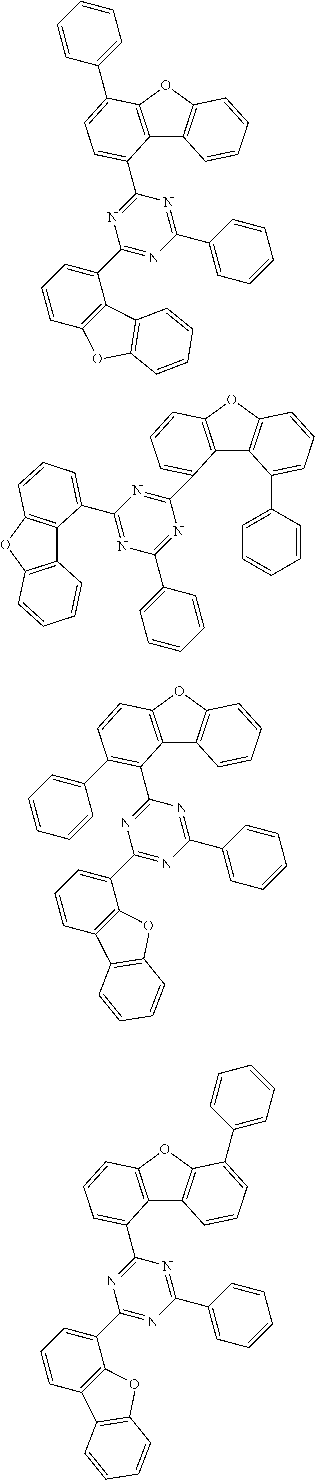

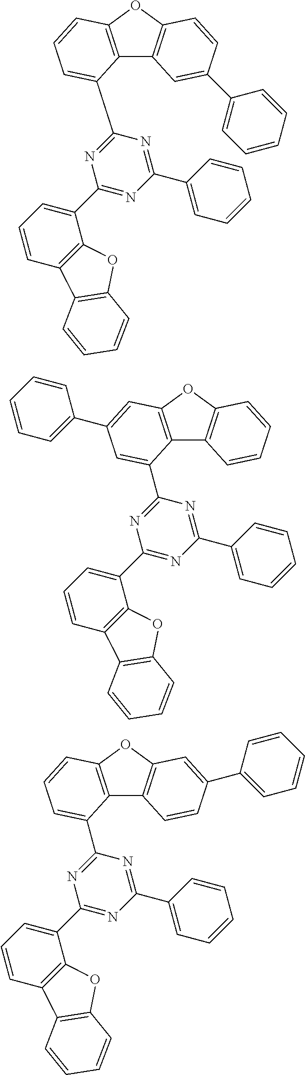

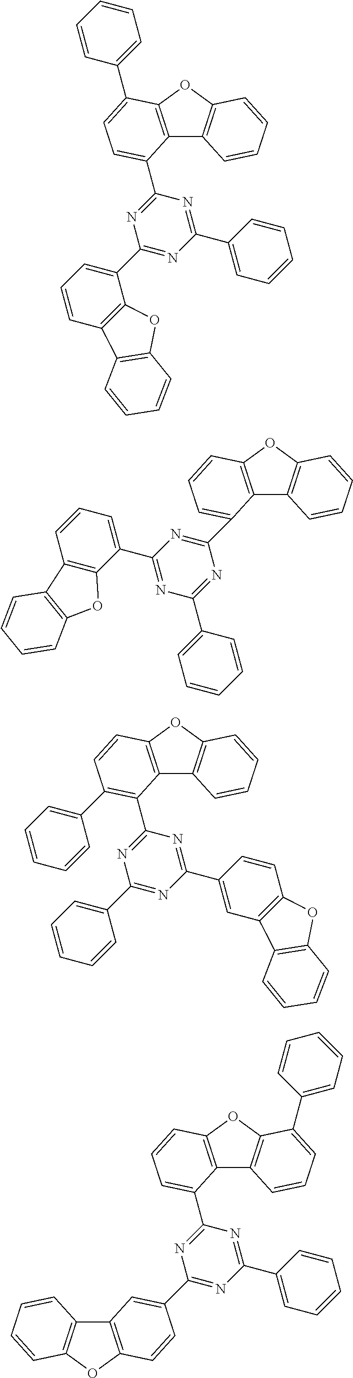

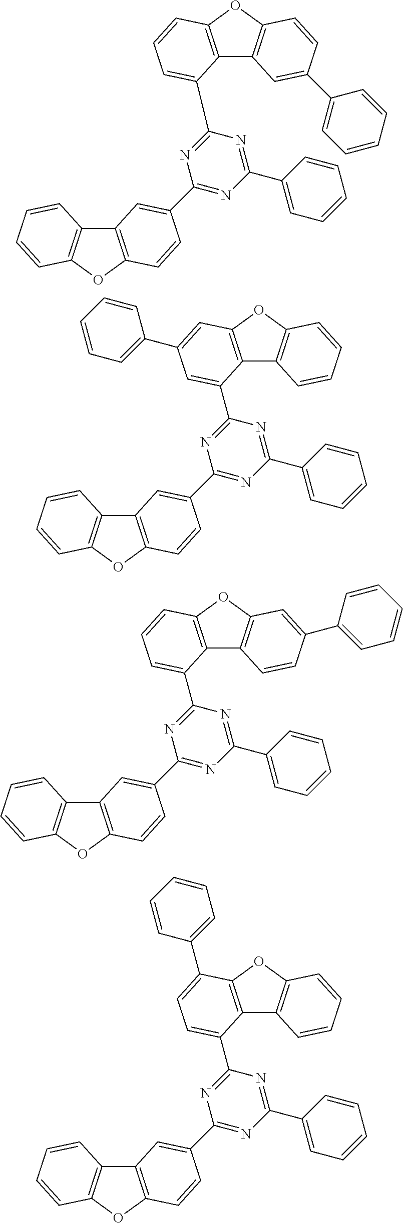

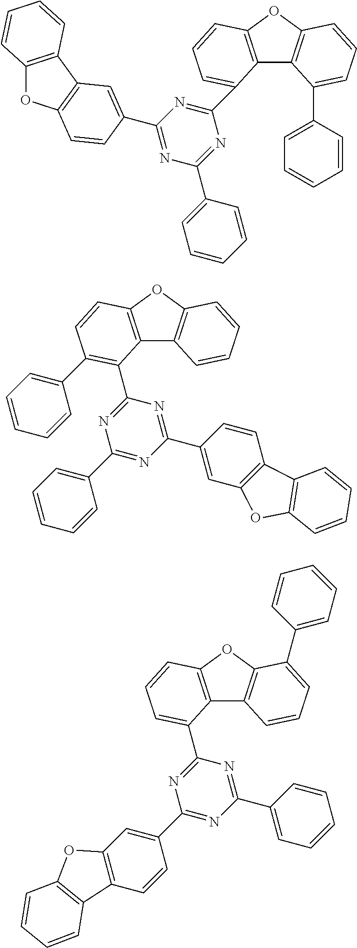

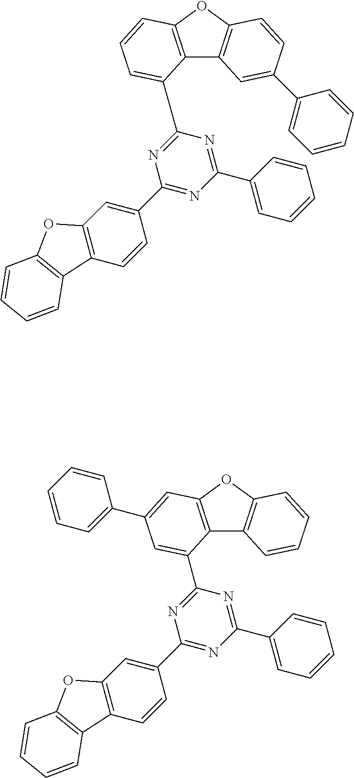 [SMILES: c1ccc(-c2ccc3oc4cccc(-c5nc(-c6ccccc6)nc(-c6ccc7c(c6)oc6ccccc67)n5)c4c3c2)cc1 ; c1ccc(-c2cc(-c3nc(-c4ccccc4)nc(-c4ccc5c(c4)oc4ccccc45)n3)c3c(c2)oc2ccccc23)cc1]

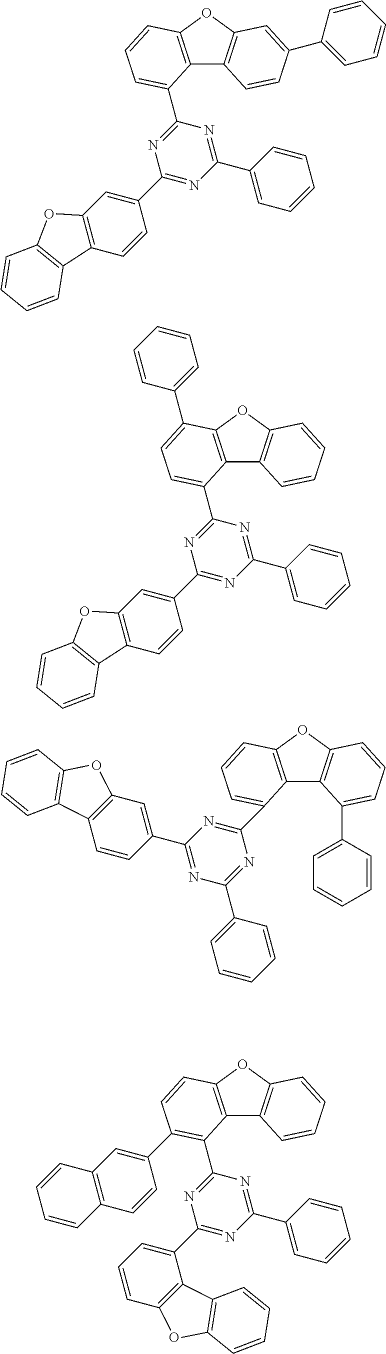 [SMILES: c1ccc(-c2ccc3c(c2)oc2cccc(-c4nc(-c5ccccc5)nc(-c5ccc6c(c5)oc5ccccc56)n4)c23)cc1 ; c1ccc(-c2nc(-c3ccc4c(c3)oc3ccccc34)nc(-c3ccc(-c4ccccc4)c4oc5ccccc5c34)n2)cc1 ; c1ccc(-c2nc(-c3ccc4c(c3)oc3ccccc34)nc(-c3cccc4oc5cccc(-c6ccccc6)c5c34)n2)cc1 ; c1ccc(-c2nc(-c3cccc4oc5ccccc5c34)nc(-c3c(-c4ccc5ccccc5c4)ccc4oc5ccccc5c34)n2)cc1]

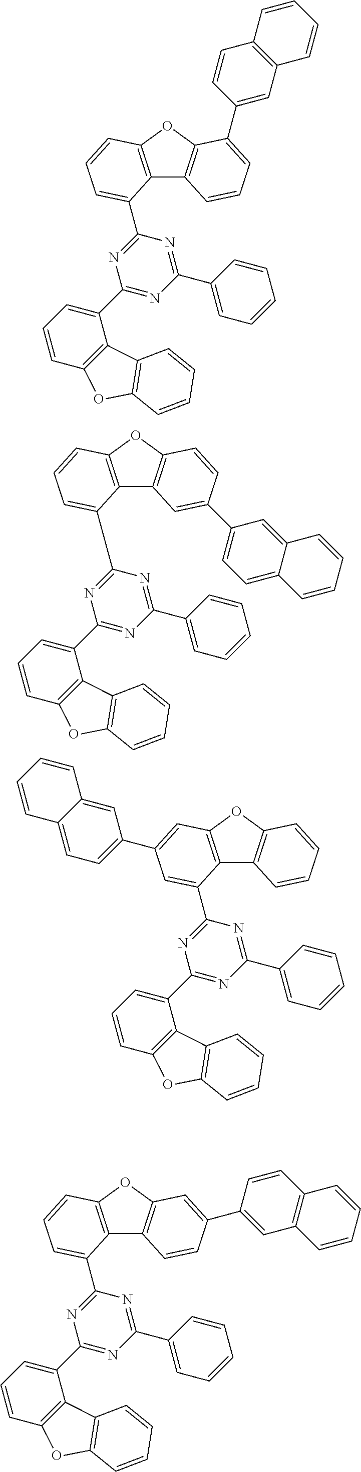

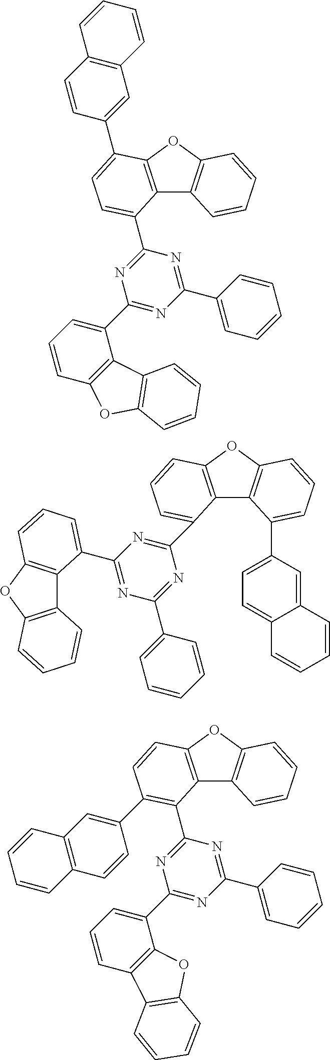

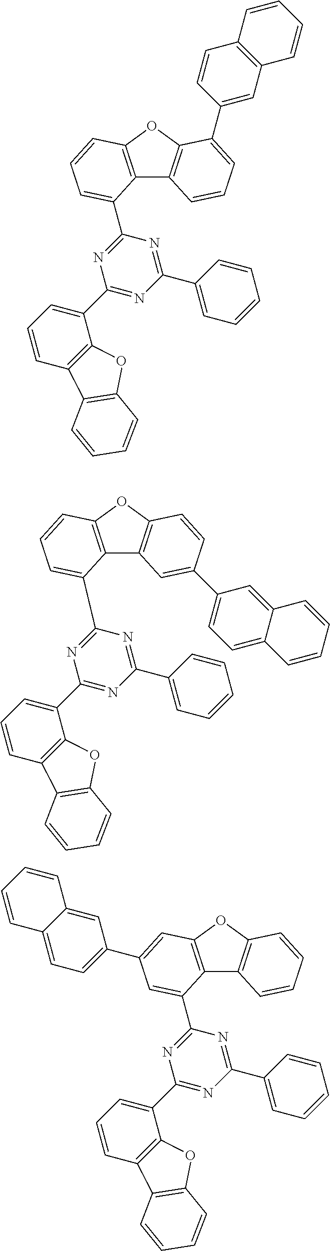

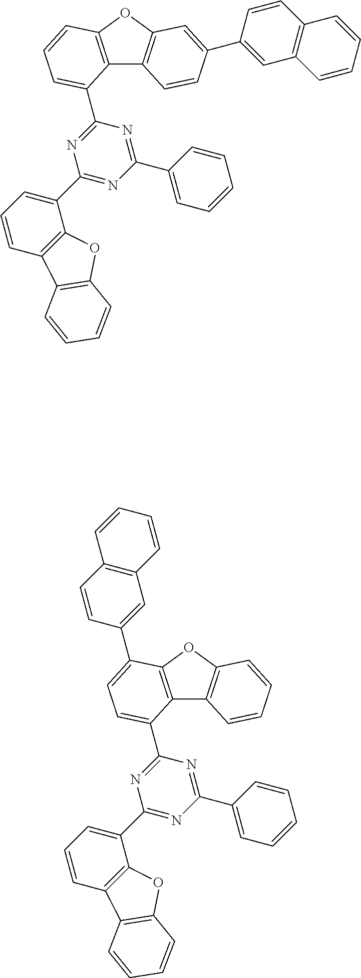 [SMILES: c1ccc(-c2nc(-c3cccc4c3oc3ccccc34)nc(-c3cccc4oc5cc(-c6ccc7ccccc7c6)ccc5c34)n2)cc1 ; c1ccc(-c2nc(-c3cccc4c3oc3ccccc34)nc(-c3ccc(-c4ccc5ccccc5c4)c4oc5ccccc5c34)n2)cc1]

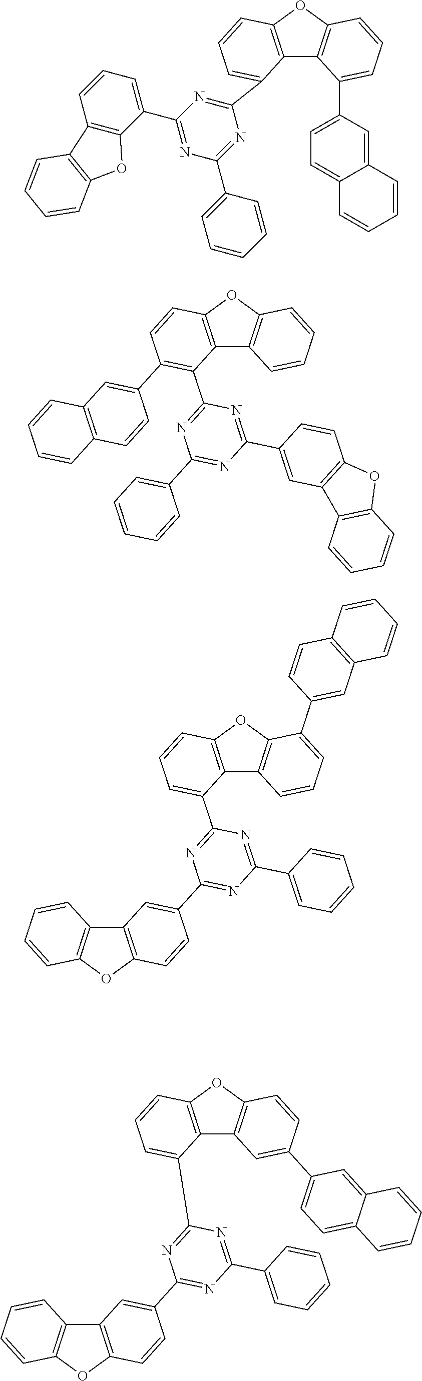

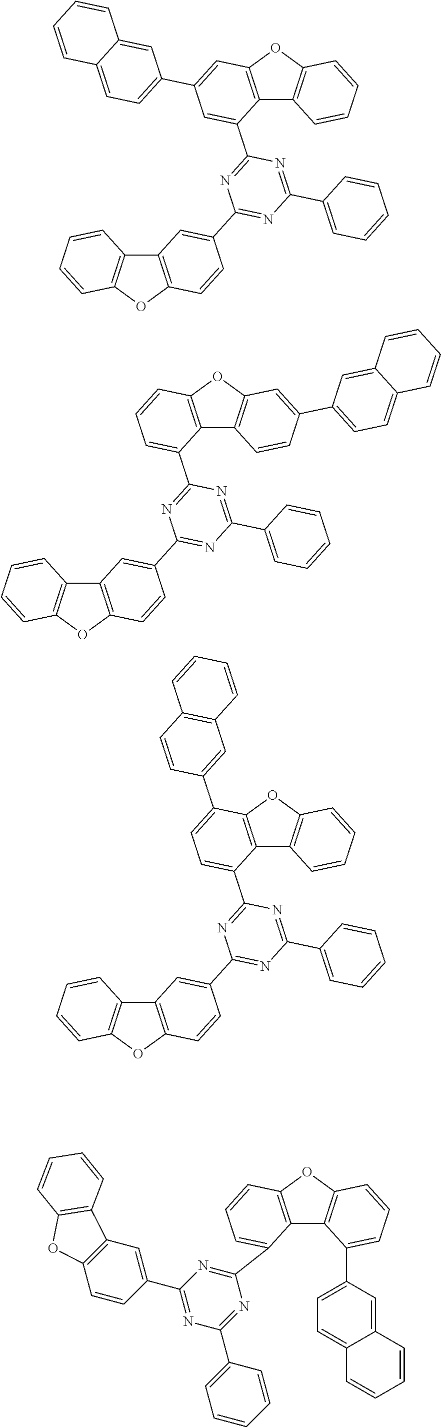 [SMILES: c1ccc(-c2nc(-c3ccc4oc5ccccc5c4c3)nc(-c3cc(-c4ccc5ccccc5c4)cc4oc5ccccc5c34)n2)cc1 ; c1ccc(-c2nc(-c3ccc4oc5ccccc5c4c3)nc(-c3cccc4oc5cc(-c6ccc7ccccc7c6)ccc5c34)n2)cc1 ; c1ccc(-c2nc(-c3ccc4oc5ccccc5c4c3)nc(-c3ccc(-c4ccc5ccccc5c4)c4oc5ccccc5c34)n2)cc1 ; c1ccc(-c2nc(-c3ccc4oc5ccccc5c4c3)nc(-c3cccc4oc5cccc(-c6ccc7ccccc7c6)c5c34)n2)cc1]

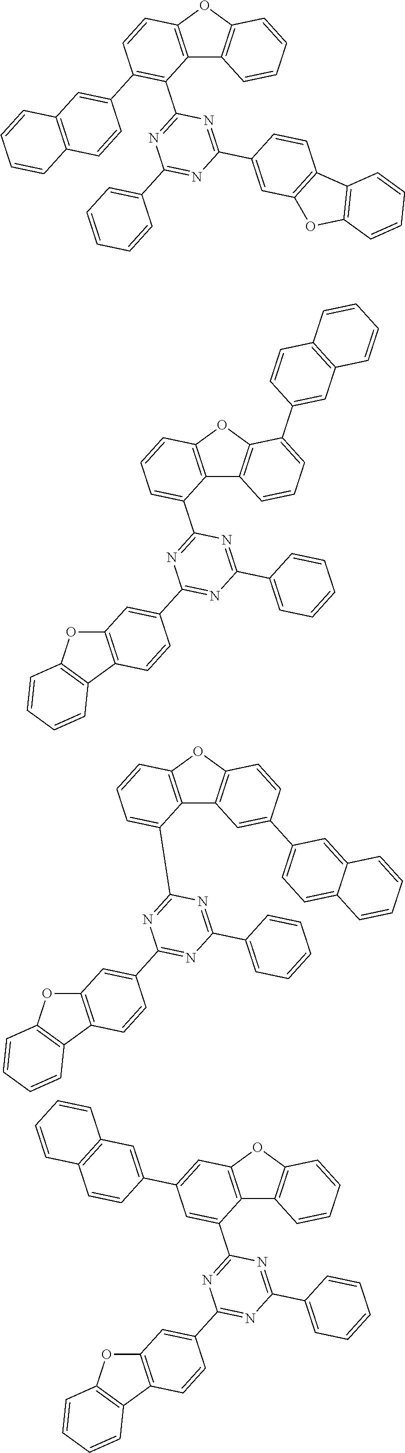

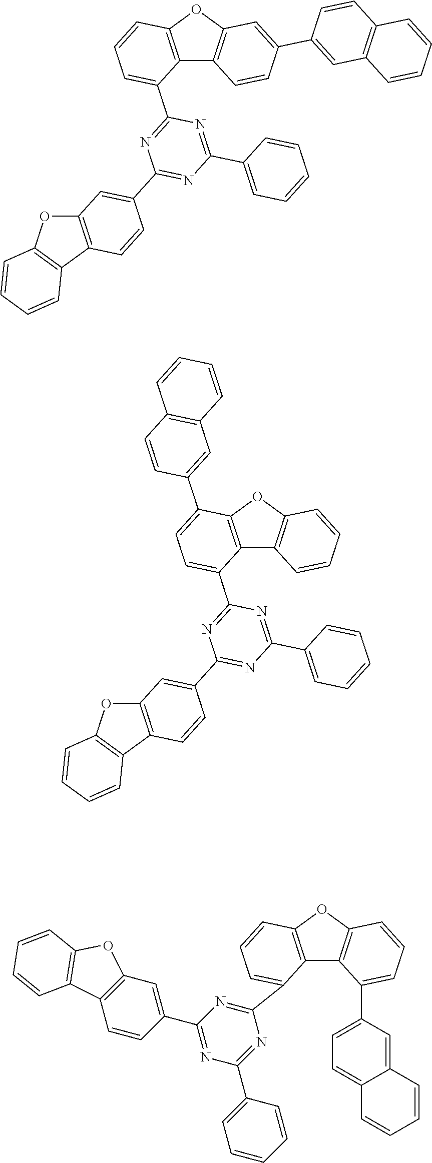 [SMILES: c1ccc(-c2nc(-c3ccc4c(c3)oc3ccccc34)nc(-c3cccc4oc5cc(-c6ccc7ccccc7c6)ccc5c34)n2)cc1 ; c1ccc(-c2nc(-c3ccc4c(c3)oc3ccccc34)nc(-c3ccc(-c4ccc5ccccc5c4)c4oc5ccccc5c34)n2)cc1 ; c1ccc(-c2nc(-c3ccc4c(c3)oc3ccccc34)nc(-c3cccc4oc5cccc(-c6ccc7ccccc7c6)c5c34)n2)cc1]

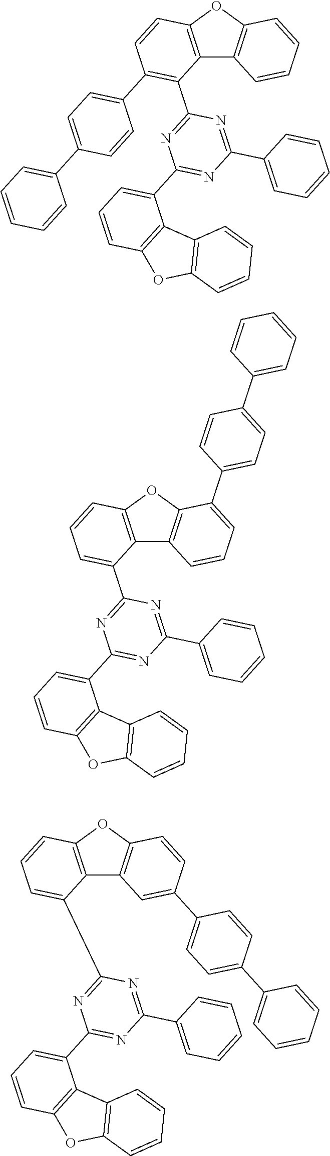

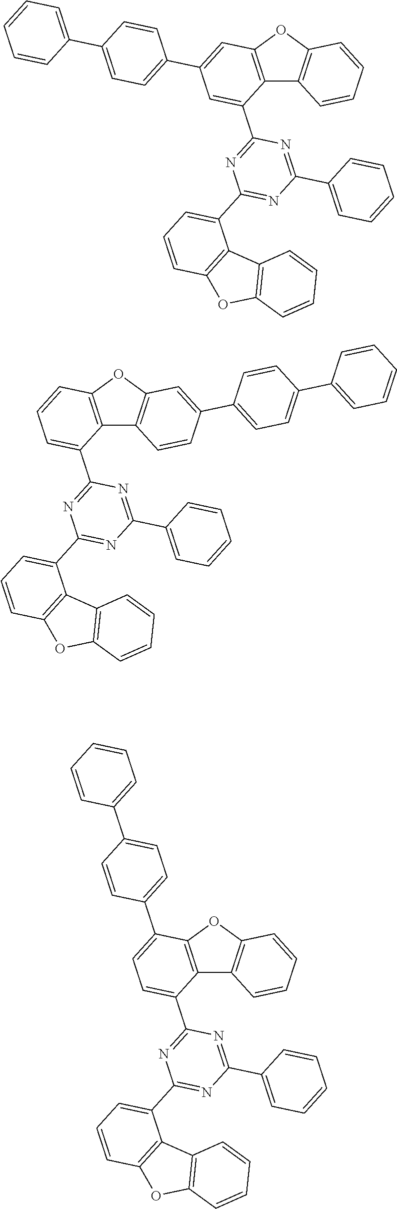 [SMILES: c1ccc(-c2ccc(-c3cc(-c4nc(-c5ccccc5)nc(-c5cccc6oc7ccccc7c56)n4)c4c(c3)oc3ccccc34)cc2)cc1 ; c1ccc(-c2ccc(-c3ccc4c(c3)oc3cccc(-c5nc(-c6ccccc6)nc(-c6cccc7oc8ccccc8c67)n5)c34)cc2)cc1 ; c1ccc(-c2ccc(-c3ccc(-c4nc(-c5ccccc5)nc(-c5cccc6oc7ccccc7c56)n4)c4c3oc3ccccc34)cc2)cc1]

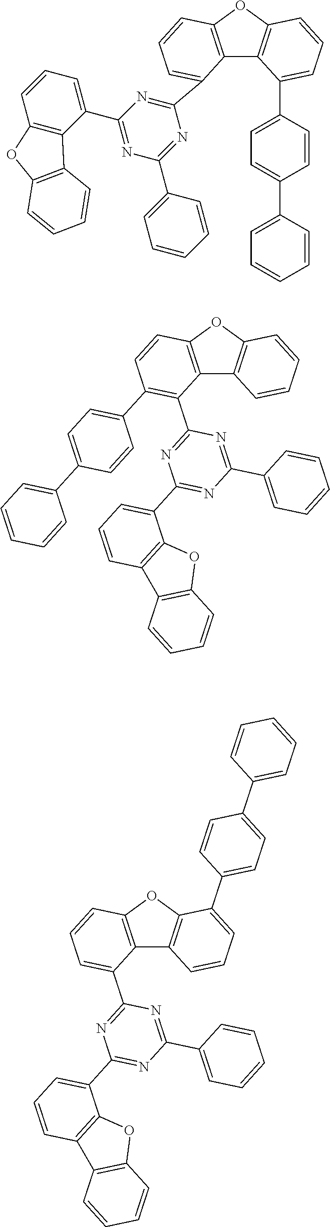 [SMILES: c1ccc(-c2ccc(-c3cccc4oc5cccc(-c6nc(-c7ccccc7)nc(-c7cccc8oc9ccccc9c78)n6)c5c34)cc2)cc1 ; c1ccc(-c2ccc(-c3ccc4oc5ccccc5c4c3-c3nc(-c4ccccc4)nc(-c4cccc5c4oc4ccccc45)n3)cc2)cc1 ; c1ccc(-c2ccc(-c3cccc4c3oc3cccc(-c5nc(-c6ccccc6)nc(-c6cccc7c6oc6ccccc67)n5)c34)cc2)cc1]

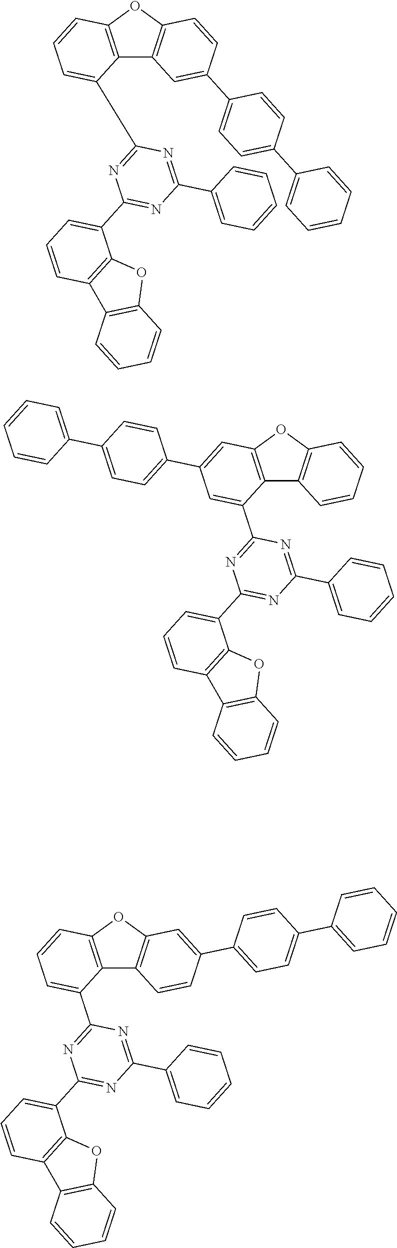 [SMILES: c1ccc(-c2ccc(-c3ccc4oc5cccc(-c6nc(-c7ccccc7)nc(-c7cccc8c7oc7ccccc78)n6)c5c4c3)cc2)cc1 ; c1ccc(-c2ccc(-c3cc(-c4nc(-c5ccccc5)nc(-c5cccc6c5oc5ccccc56)n4)c4c(c3)oc3ccccc34)cc2)cc1 ; c1ccc(-c2ccc(-c3ccc4c(c3)oc3cccc(-c5nc(-c6ccccc6)nc(-c6cccc7c6oc6ccccc67)n5)c34)cc2)cc1]

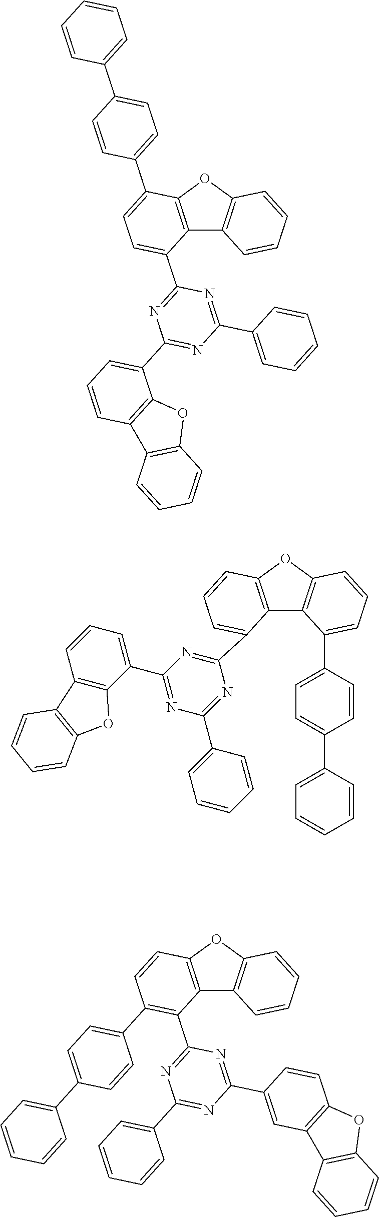 [SMILES: c1ccc(-c2ccc(-c3ccc(-c4nc(-c5ccccc5)nc(-c5cccc6c5oc5ccccc56)n4)c4c3oc3ccccc34)cc2)cc1 ; c1ccc(-c2ccc(-c3cccc4oc5cccc(-c6nc(-c7ccccc7)nc(-c7cccc8c7oc7ccccc78)n6)c5c34)cc2)cc1 ; c1ccc(-c2ccc(-c3ccc4oc5ccccc5c4c3-c3nc(-c4ccccc4)nc(-c4ccc5oc6ccccc6c5c4)n3)cc2)cc1]

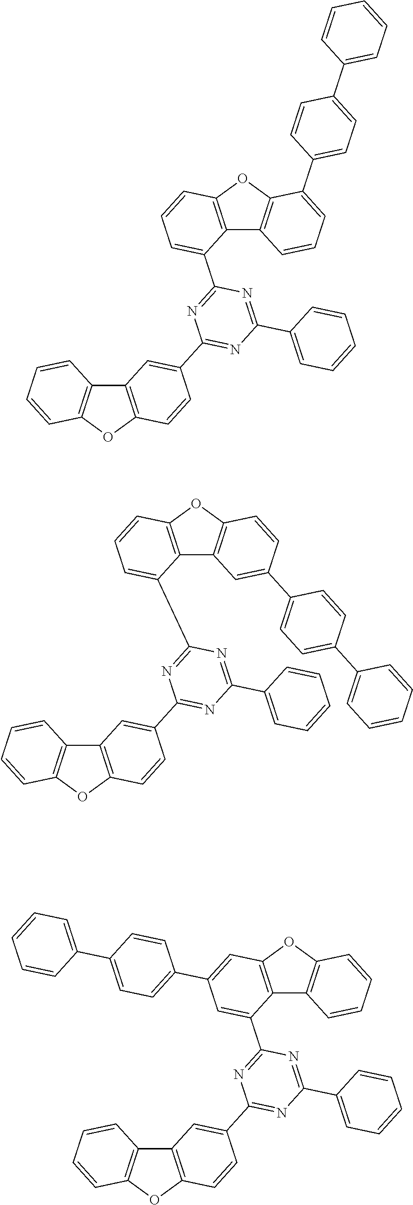 [SMILES: c1ccc(-c2ccc(-c3cccc4c3oc3cccc(-c5nc(-c6ccccc6)nc(-c6ccc7oc8ccccc8c7c6)n5)c34)cc2)cc1 ; c1ccc(-c2ccc(-c3ccc4oc5cccc(-c6nc(-c7ccccc7)nc(-c7ccc8oc9ccccc9c8c7)n6)c5c4c3)cc2)cc1 ; c1ccc(-c2ccc(-c3cc(-c4nc(-c5ccccc5)nc(-c5ccc6oc7ccccc7c6c5)n4)c4c(c3)oc3ccccc34)cc2)cc1]

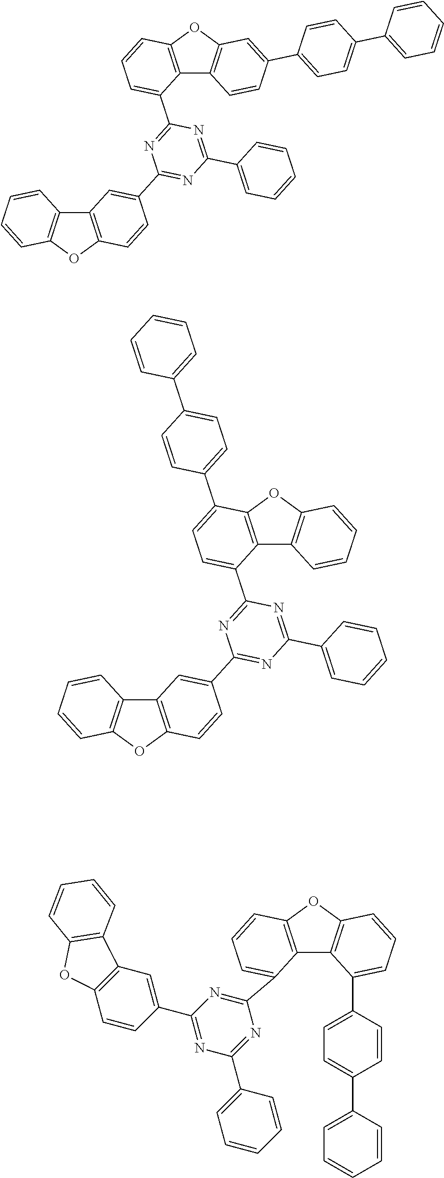 [SMILES: c1ccc(-c2ccc(-c3ccc4c(c3)oc3cccc(-c5nc(-c6ccccc6)nc(-c6ccc7oc8ccccc8c7c6)n5)c34)cc2)cc1 ; c1ccc(-c2ccc(-c3ccc(-c4nc(-c5ccccc5)nc(-c5ccc6oc7ccccc7c6c5)n4)c4c3oc3ccccc34)cc2)cc1 ; c1ccc(-c2ccc(-c3cccc4oc5cccc(-c6nc(-c7ccccc7)nc(-c7ccc8oc9ccccc9c8c7)n6)c5c34)cc2)cc1]

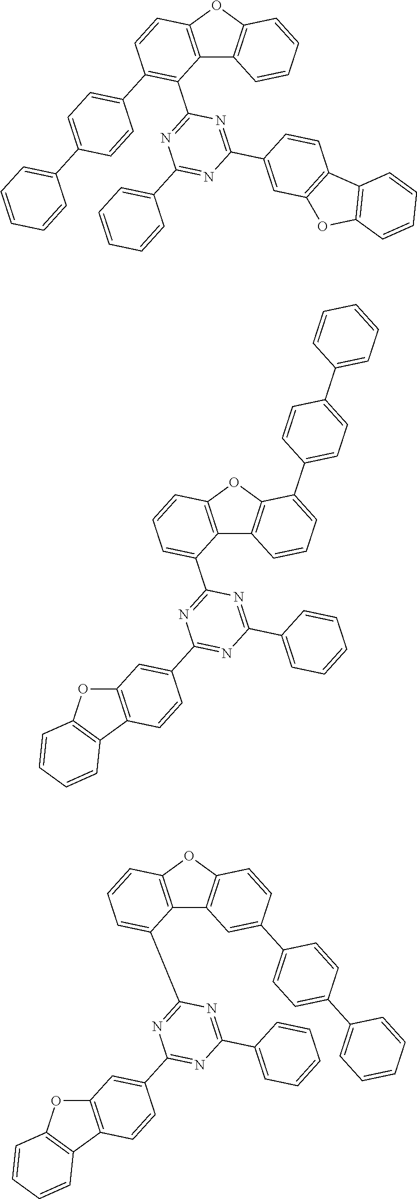 [SMILES: c1ccc(-c2ccc(-c3ccc4oc5ccccc5c4c3-c3nc(-c4ccccc4)nc(-c4ccc5c(c4)oc4ccccc45)n3)cc2)cc1 ; c1ccc(-c2ccc(-c3cccc4c3oc3cccc(-c5nc(-c6ccccc6)nc(-c6ccc7c(c6)oc6ccccc67)n5)c34)cc2)cc1 ; c1ccc(-c2ccc(-c3ccc4oc5cccc(-c6nc(-c7ccccc7)nc(-c7ccc8c(c7)oc7ccccc78)n6)c5c4c3)cc2)cc1]

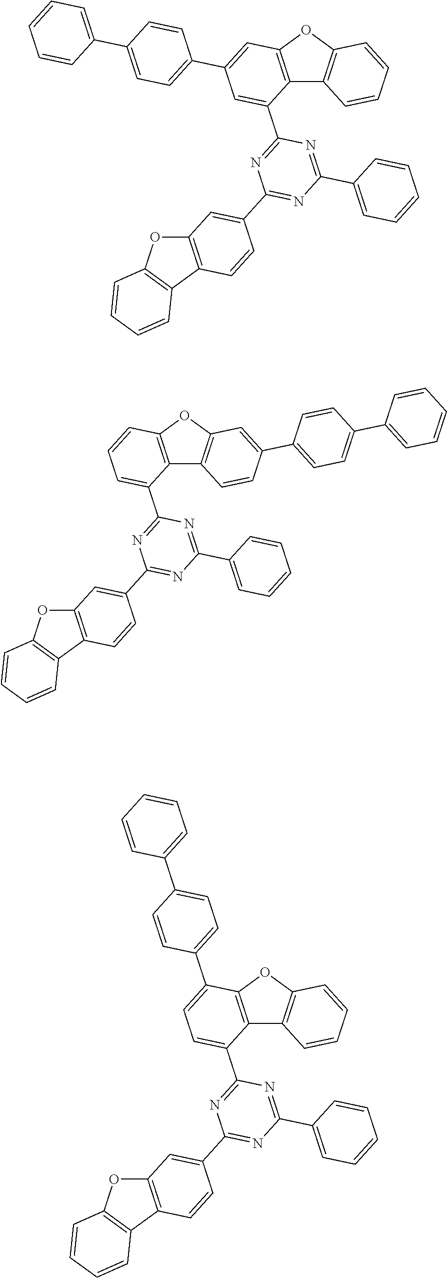 [SMILES: c1ccc(-c2ccc(-c3cc(-c4nc(-c5ccccc5)nc(-c5ccc6c(c5)oc5ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc7c(c6)oc6ccccc67)n5)c34)cc2)cc1 ; c1ccc(-c2ccc(-c3ccc(-c4nc(-c5ccccc5)nc(-c5ccc6c(c5)oc5ccccc56)n4)c4c3oc3ccccc34)cc2)cc1]

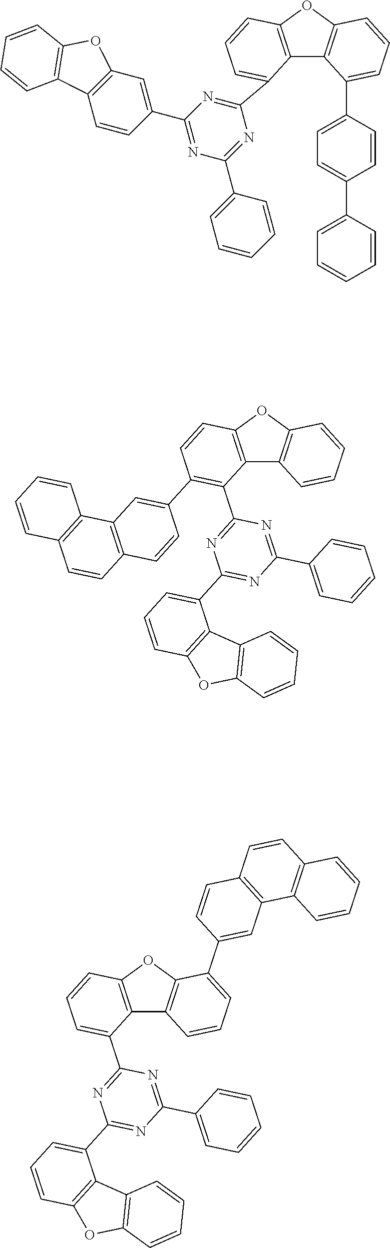 [SMILES: c1ccc(-c2ccc(-c3cccc4oc5cccc(-c6nc(-c7ccccc7)nc(-c7ccc8c(c7)oc7ccccc78)n6)c5c34)cc2)cc1 ; c1ccc(-c2nc(-c3cccc4oc5ccccc5c34)nc(-c3c(-c4ccc5ccc6ccccc6c5c4)ccc4oc5ccccc5c34)n2)cc1 ; c1ccc(-c2nc(-c3cccc4oc5ccccc5c34)nc(-c3cccc4oc5c(-c6ccc7ccc8ccccc8c7c6)cccc5c34)n2)cc1]

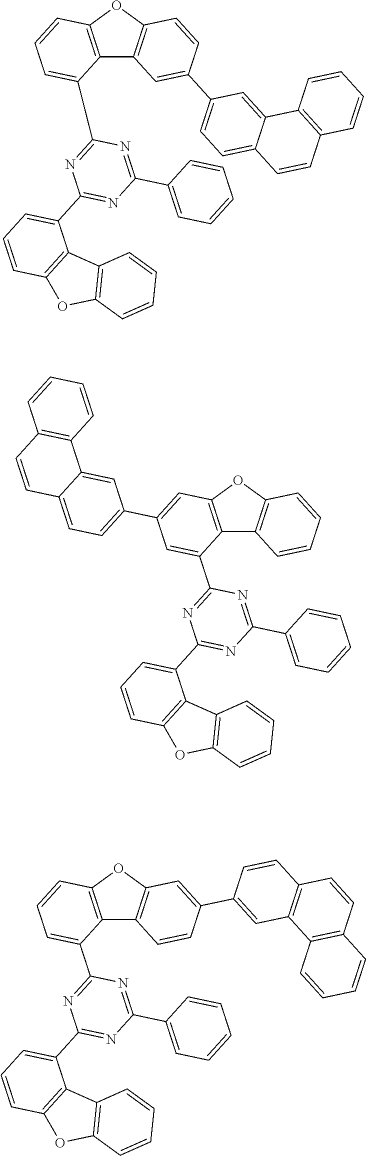 [SMILES: c1ccc(-c2nc(-c3cccc4oc5ccccc5c34)nc(-c3cccc4oc5ccc(-c6ccc7ccc8ccccc8c7c6)cc5c34)n2)cc1 ; c1ccc(-c2nc(-c3cccc4oc5ccccc5c34)nc(-c3cc(-c4ccc5ccc6ccccc6c5c4)cc4oc5ccccc5c34)n2)cc1 ; c1ccc(-c2nc(-c3cccc4oc5ccccc5c34)nc(-c3cccc4oc5cc(-c6ccc7ccc8ccccc8c7c6)ccc5c34)n2)cc1]

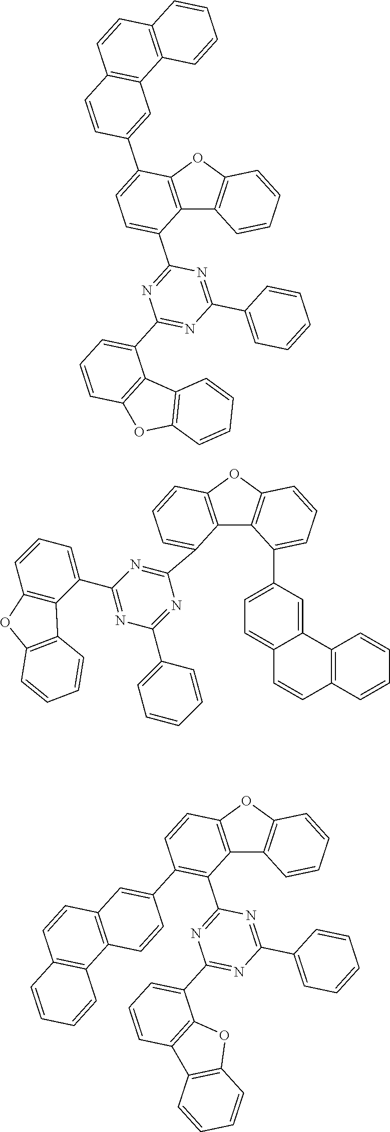

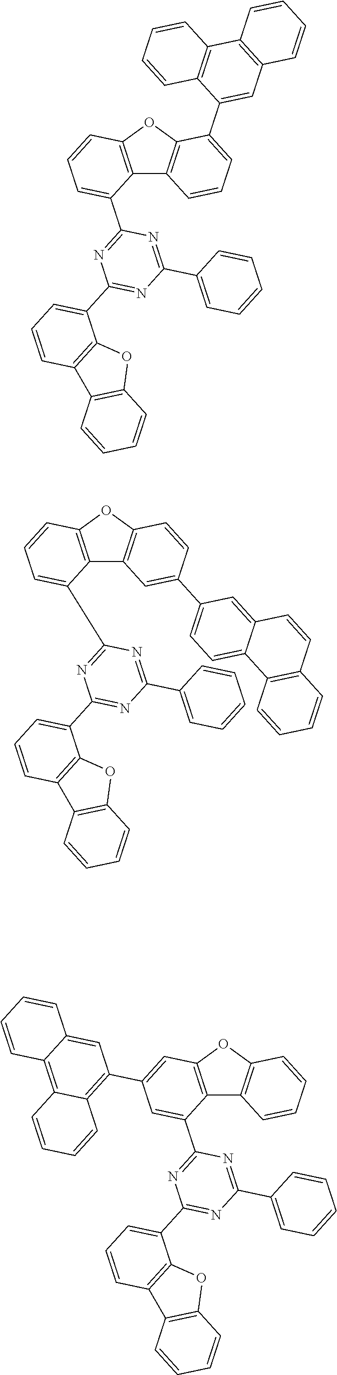 [SMILES: c1ccc(-c2nc(-c3cccc4c3oc3ccccc34)nc(-c3cccc4oc5c(-c6cc7ccccc7c7ccccc67)cccc5c34)n2)cc1 ; c1ccc(-c2nc(-c3cccc4c3oc3ccccc34)nc(-c3cccc4oc5ccc(-c6ccc7c(ccc8ccccc87)c6)cc5c34)n2)cc1 ; c1ccc(-c2nc(-c3cccc4c3oc3ccccc34)nc(-c3cc(-c4cc5ccccc5c5ccccc45)cc4oc5ccccc5c34)n2)cc1]

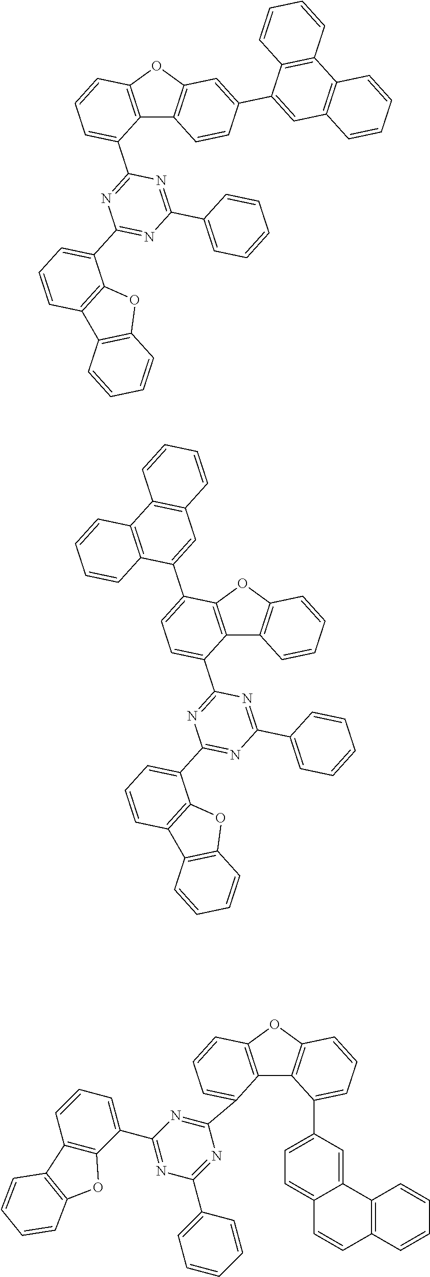 [SMILES: c1ccc(-c2nc(-c3cccc4c3oc3ccccc34)nc(-c3cccc4oc5cc(-c6cc7ccccc7c7ccccc67)ccc5c34)n2)cc1 ; c1ccc(-c2nc(-c3cccc4c3oc3ccccc34)nc(-c3ccc(-c4cc5ccccc5c5ccccc45)c4oc5ccccc5c34)n2)cc1 ; c1ccc(-c2nc(-c3cccc4c3oc3ccccc34)nc(-c3cccc4oc5cccc(-c6ccc7ccc8ccccc8c7c6)c5c34)n2)cc1]

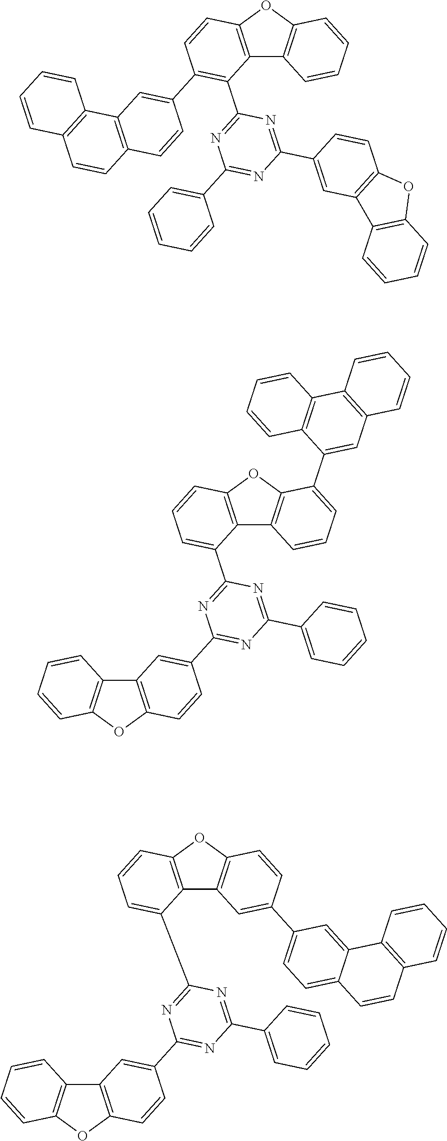 [SMILES: c1ccc(-c2nc(-c3ccc4oc5ccccc5c4c3)nc(-c3c(-c4ccc5ccc6ccccc6c5c4)ccc4oc5ccccc5c34)n2)cc1 ; c1ccc(-c2nc(-c3ccc4oc5ccccc5c4c3)nc(-c3cccc4oc5c(-c6cc7ccccc7c7ccccc67)cccc5c34)n2)cc1 ; c1ccc(-c2nc(-c3ccc4oc5ccccc5c4c3)nc(-c3cccc4oc5ccc(-c6ccc7ccc8ccccc8c7c6)cc5c34)n2)cc1]

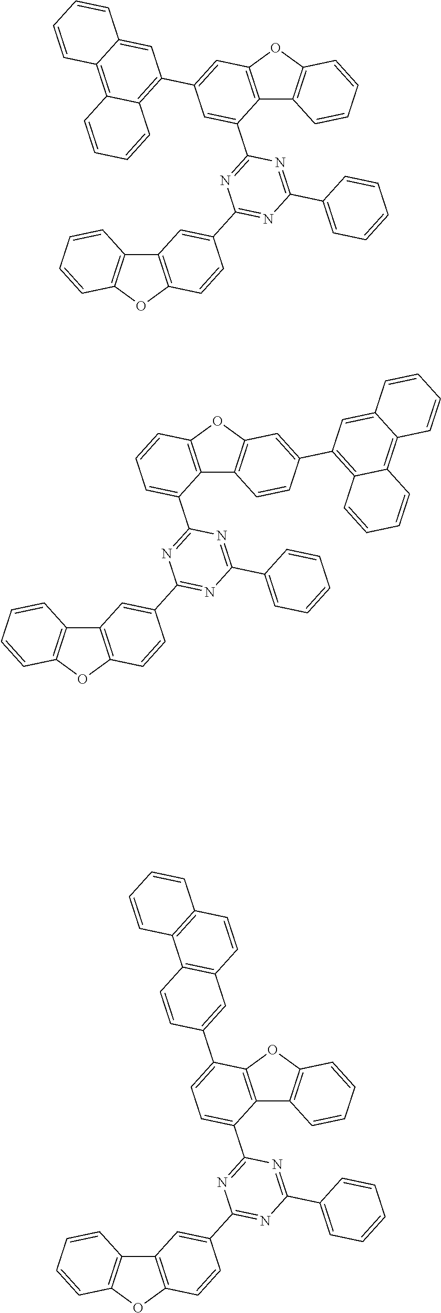 [SMILES: c1ccc(-c2nc(-c3ccc4oc5ccccc5c4c3)nc(-c3cc(-c4cc5ccccc5c5ccccc45)cc4oc5ccccc5c34)n2)cc1 ; c1ccc(-c2nc(-c3ccc4oc5ccccc5c4c3)nc(-c3cccc4oc5cc(-c6cc7ccccc7c7ccccc67)ccc5c34)n2)cc1 ; c1ccc(-c2nc(-c3ccc4oc5ccccc5c4c3)nc(-c3ccc(-c4ccc5c(ccc6ccccc65)c4)c4oc5ccccc5c34)n2)cc1]

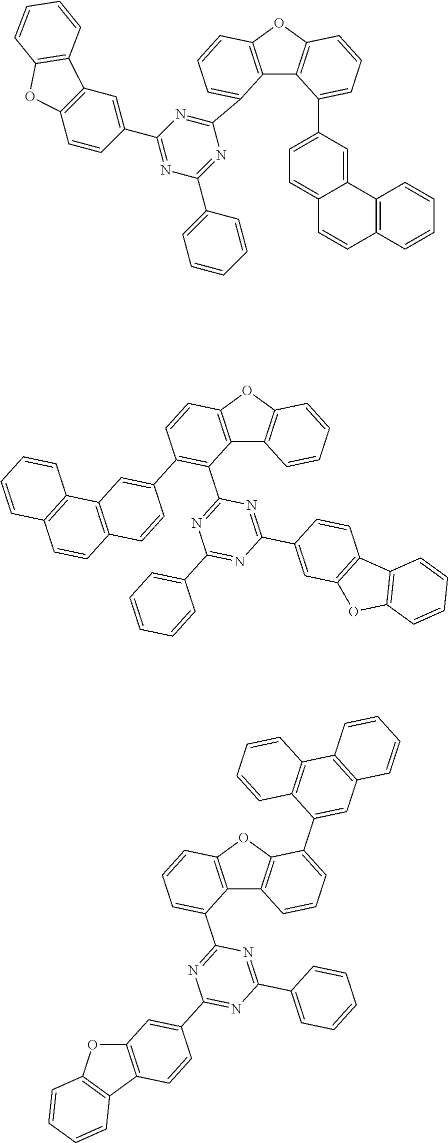 [SMILES: c1ccc(-c2nc(-c3ccc4oc5ccccc5c4c3)nc(-c3cccc4oc5cccc(-c6ccc7ccc8ccccc8c7c6)c5c34)n2)cc1 ; c1ccc(-c2nc(-c3ccc4c(c3)oc3ccccc34)nc(-c3c(-c4ccc5ccc6ccccc6c5c4)ccc4oc5ccccc5c34)n2)cc1 ; c1ccc(-c2nc(-c3ccc4c(c3)oc3ccccc34)nc(-c3cccc4oc5c(-c6cc7ccccc7c7ccccc67)cccc5c34)n2)cc1]

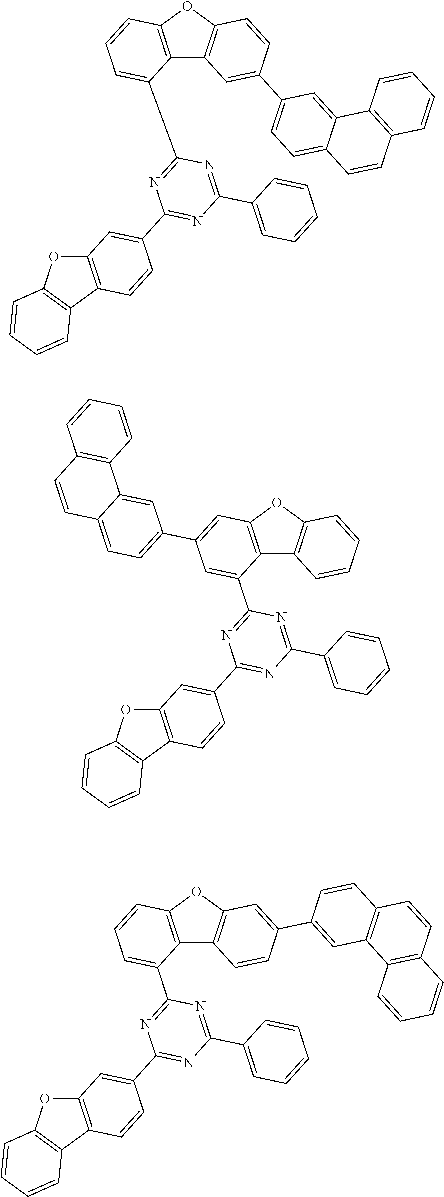 [SMILES: c1ccc(-c2nc(-c3ccc4c(c3)oc3ccccc34)nc(-c3cccc4oc5ccc(-c6ccc7ccc8ccccc8c7c6)cc5c34)n2)cc1 ; c1ccc(-c2nc(-c3ccc4c(c3)oc3ccccc34)nc(-c3cc(-c4ccc5ccc6ccccc6c5c4)cc4oc5ccccc5c34)n2)cc1 ; c1ccc(-c2nc(-c3ccc4c(c3)oc3ccccc34)nc(-c3cccc4oc5cc(-c6ccc7ccc8ccccc8c7c6)ccc5c34)n2)cc1]

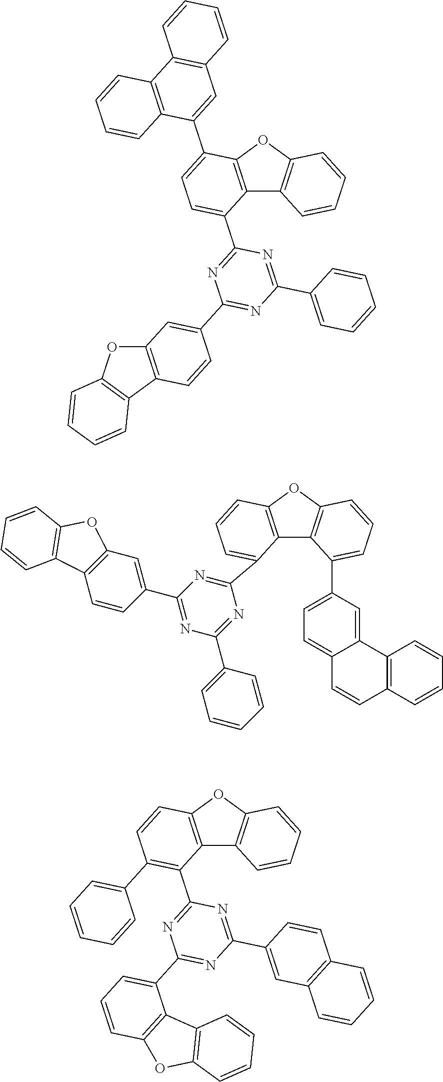 [SMILES: c1ccc(-c2nc(-c3ccc4c(c3)oc3ccccc34)nc(-c3ccc(-c4cc5ccccc5c5ccccc45)c4oc5ccccc5c34)n2)cc1 ; c1ccc(-c2nc(-c3ccc4c(c3)oc3ccccc34)nc(-c3cccc4oc5cccc(-c6ccc7ccc8ccccc8c7c6)c5c34)n2)cc1 ; c1ccc(-c2ccc3oc4ccccc4c3c2-c2nc(-c3ccc4ccccc4c3)nc(-c3cccc4oc5ccccc5c34)n2)cc1]

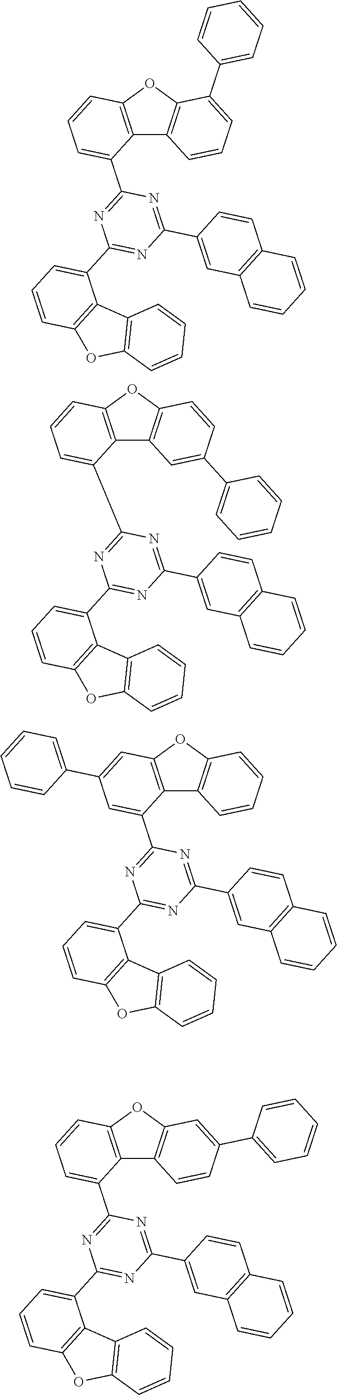

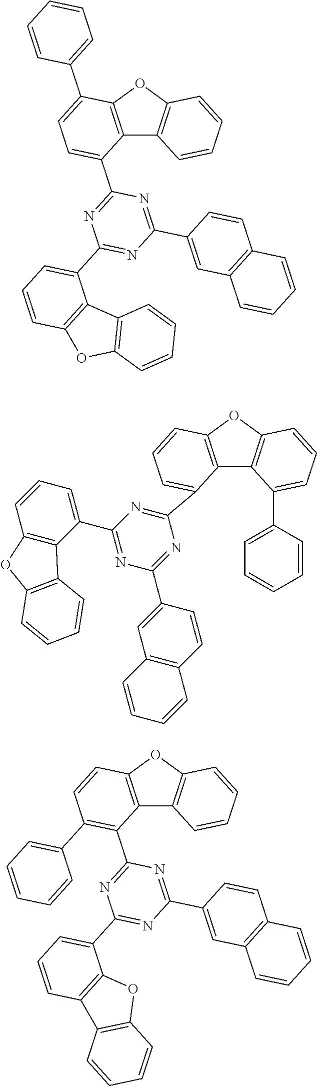

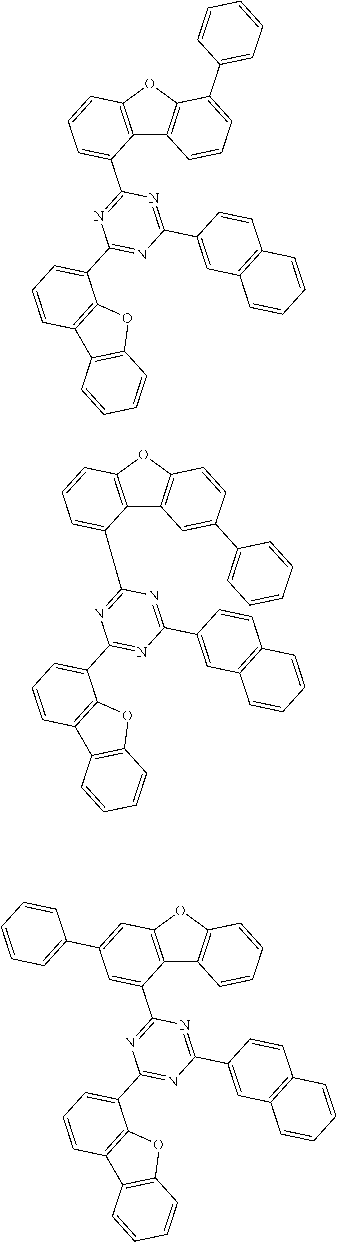 [SMILES: c1ccc(-c2cccc3c2oc2cccc(-c4nc(-c5ccc6ccccc6c5)nc(-c5cccc6c5oc5ccccc56)n4)c23)cc1 ; c1ccc(-c2ccc3oc4cccc(-c5nc(-c6ccc7ccccc7c6)nc(-c6cccc7c6oc6ccccc67)n5)c4c3c2)cc1 ; c1ccc(-c2cc(-c3nc(-c4ccc5ccccc5c4)nc(-c4cccc5c4oc4ccccc45)n3)c3c(c2)oc2ccccc23)cc1]

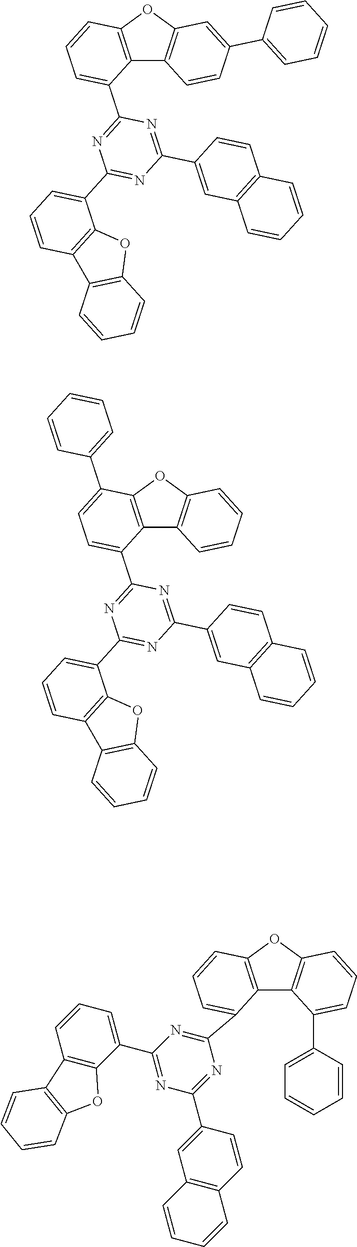 [SMILES: c1ccc(-c2ccc3c(c2)oc2cccc(-c4nc(-c5ccc6ccccc6c5)nc(-c5cccc6c5oc5ccccc56)n4)c23)cc1 ; c1ccc(-c2ccc(-c3nc(-c4ccc5ccccc5c4)nc(-c4cccc5c4oc4ccccc45)n3)c3c2oc2ccccc23)cc1 ; c1ccc(-c2cccc3oc4cccc(-c5nc(-c6ccc7ccccc7c6)nc(-c6cccc7c6oc6ccccc67)n5)c4c23)cc1]

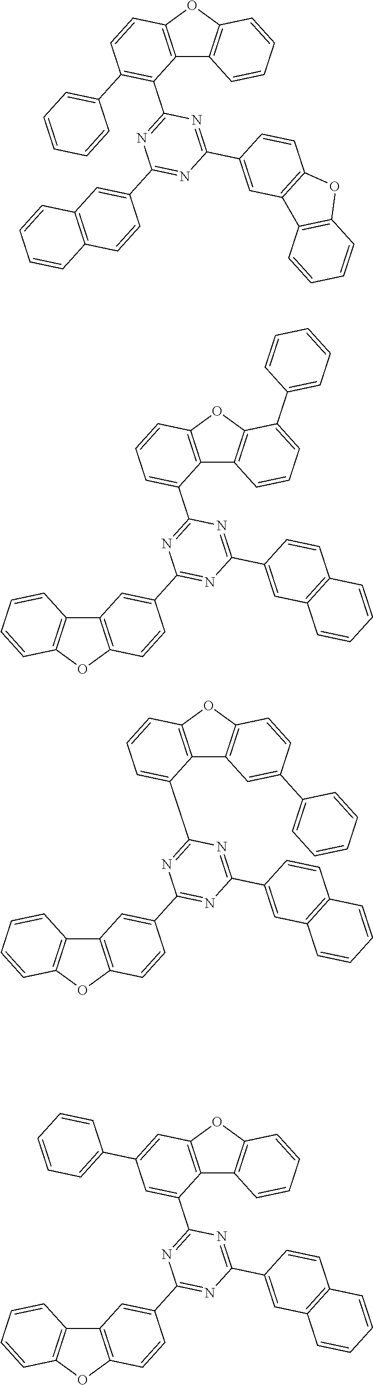

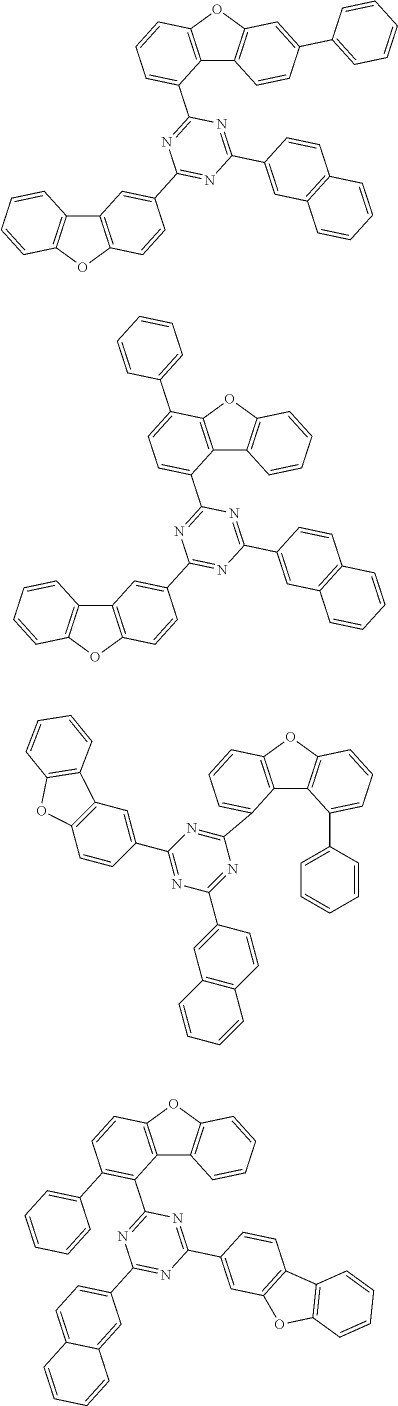 [SMILES: c1ccc(-c2ccc3c(c2)oc2cccc(-c4nc(-c5ccc6ccccc6c5)nc(-c5ccc6oc7ccccc7c6c5)n4)c23)cc1 ; c1ccc(-c2ccc(-c3nc(-c4ccc5ccccc5c4)nc(-c4ccc5oc6ccccc6c5c4)n3)c3c2oc2ccccc23)cc1 ; c1ccc(-c2cccc3oc4cccc(-c5nc(-c6ccc7ccccc7c6)nc(-c6ccc7oc8ccccc8c7c6)n5)c4c23)cc1 ; c1ccc(-c2ccc3oc4ccccc4c3c2-c2nc(-c3ccc4ccccc4c3)nc(-c3ccc4c(c3)oc3ccccc34)n2)cc1]

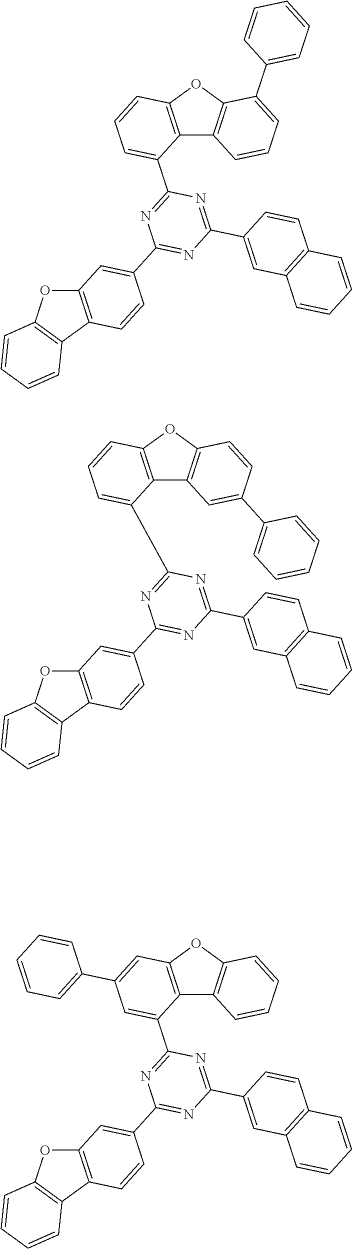 [SMILES: c1ccc(-c2cccc3c2oc2cccc(-c4nc(-c5ccc6ccccc6c5)nc(-c5ccc6c(c5)oc5ccccc56)n4)c23)cc1 ; c1ccc(-c2ccc3oc4cccc(-c5nc(-c6ccc7ccccc7c6)nc(-c6ccc7c(c6)oc6ccccc67)n5)c4c3c2)cc1 ; c1ccc(-c2cc(-c3nc(-c4ccc5ccccc5c4)nc(-c4ccc5c(c4)oc4ccccc45)n3)c3c(c2)oc2ccccc23)cc1]

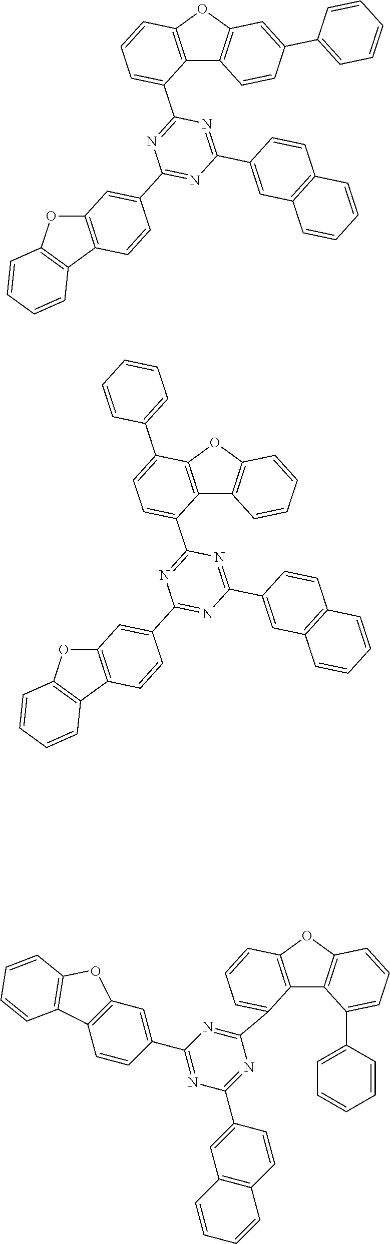 [SMILES: c1ccc(-c2ccc3c(c2)oc2cccc(-c4nc(-c5ccc6ccccc6c5)nc(-c5ccc6c(c5)oc5ccccc56)n4)c23)cc1 ; c1ccc(-c2ccc(-c3nc(-c4ccc5ccccc5c4)nc(-c4ccc5c(c4)oc4ccccc45)n3)c3c2oc2ccccc23)cc1 ; c1ccc(-c2cccc3oc4cccc(-c5nc(-c6ccc7ccccc7c6)nc(-c6ccc7c(c6)oc6ccccc67)n5)c4c23)cc1]

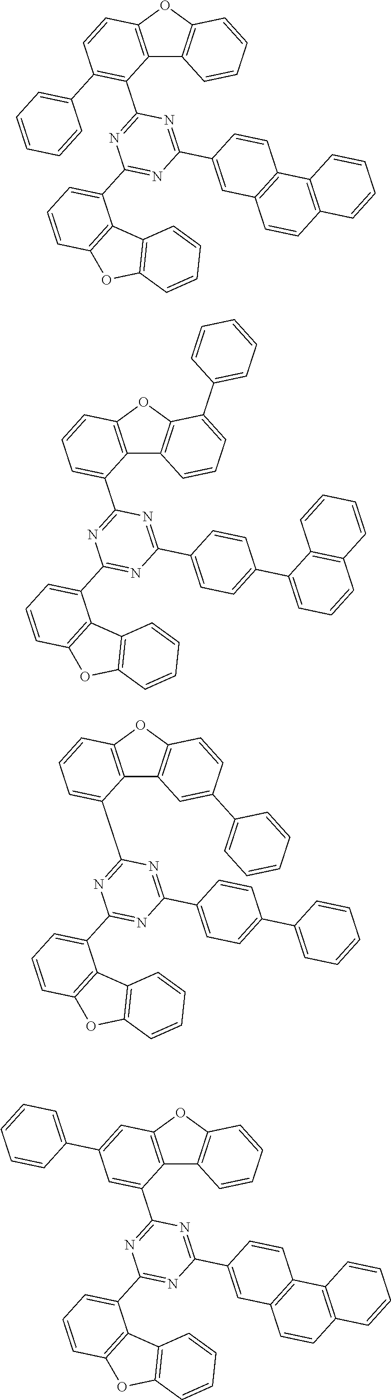

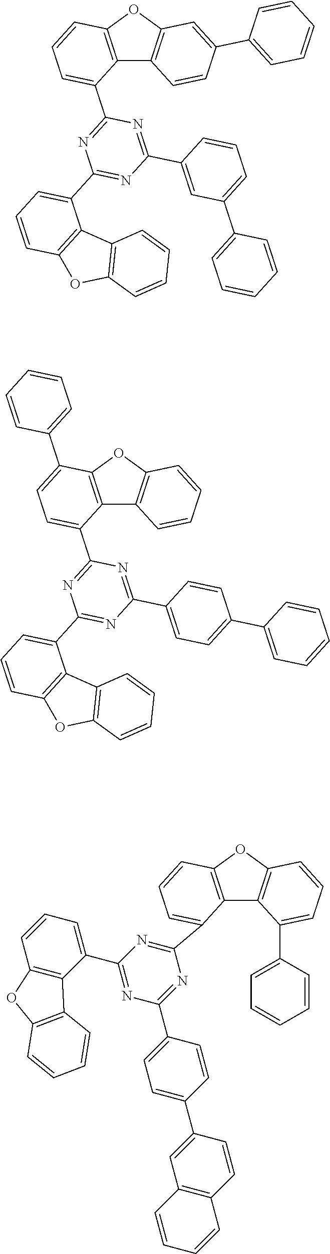 [SMILES: c1ccc(-c2cccc(-c3nc(-c4cccc5oc6ccccc6c45)nc(-c4cccc5oc6cc(-c7ccccc7)ccc6c45)n3)c2)cc1 ; c1ccc(-c2ccc(-c3nc(-c4cccc5oc6ccccc6c45)nc(-c4ccc(-c5ccccc5)c5oc6ccccc6c45)n3)cc2)cc1 ; c1ccc(-c2cccc3oc4cccc(-c5nc(-c6ccc(-c7ccc8ccccc8c7)cc6)nc(-c6cccc7oc8ccccc8c67)n5)c4c23)cc1]

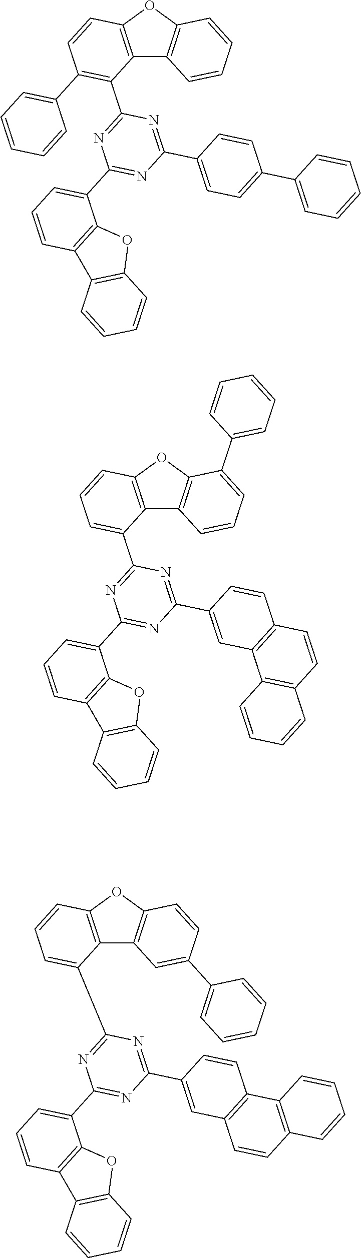 [SMILES: c1ccc(-c2ccc(-c3nc(-c4cccc5c4oc4ccccc45)nc(-c4c(-c5ccccc5)ccc5oc6ccccc6c45)n3)cc2)cc1 ; c1ccc(-c2cccc3c2oc2cccc(-c4nc(-c5ccc6ccc7ccccc7c6c5)nc(-c5cccc6c5oc5ccccc56)n4)c23)cc1 ; c1ccc(-c2ccc3oc4cccc(-c5nc(-c6ccc7c(ccc8ccccc87)c6)nc(-c6cccc7c6oc6ccccc67)n5)c4c3c2)cc1]

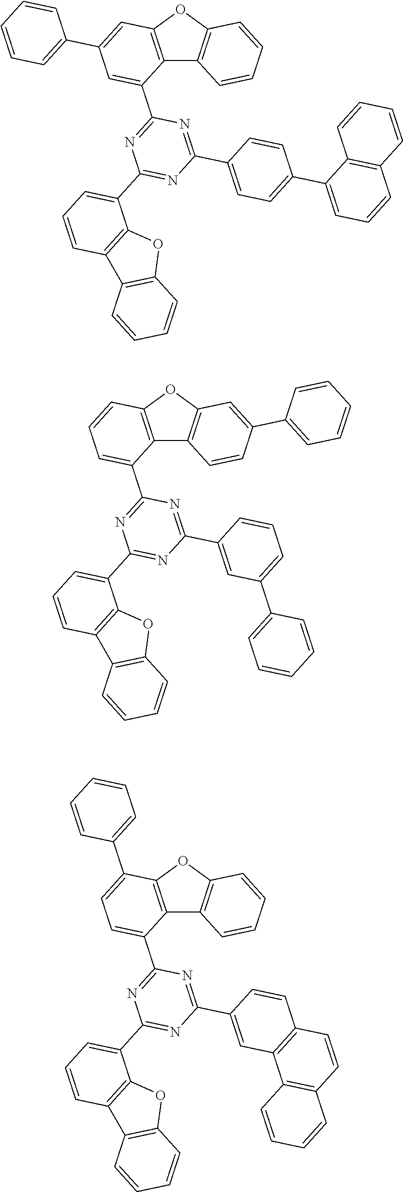 [SMILES: c1ccc(-c2cc(-c3nc(-c4ccc(-c5cccc6ccccc56)cc4)nc(-c4cccc5c4oc4ccccc45)n3)c3c(c2)oc2ccccc23)cc1 ; c1ccc(-c2cccc(-c3nc(-c4cccc5c4oc4ccccc45)nc(-c4cccc5oc6cc(-c7ccccc7)ccc6c45)n3)c2)cc1 ; c1ccc(-c2ccc(-c3nc(-c4ccc5ccc6ccccc6c5c4)nc(-c4cccc5c4oc4ccccc45)n3)c3c2oc2ccccc23)cc1]

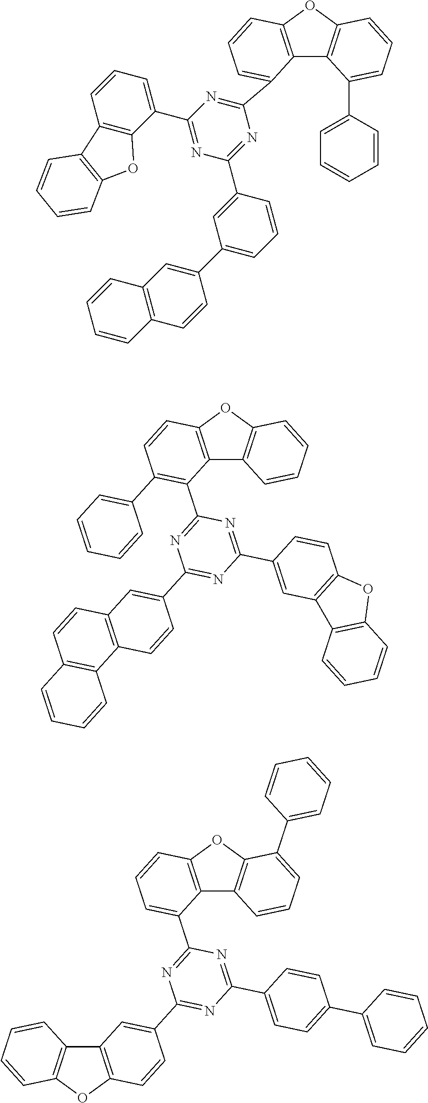 [SMILES: c1ccc(-c2cccc3oc4cccc(-c5nc(-c6cccc(-c7ccc8ccccc8c7)c6)nc(-c6cccc7c6oc6ccccc67)n5)c4c23)cc1 ; c1ccc(-c2ccc3oc4ccccc4c3c2-c2nc(-c3ccc4c(ccc5ccccc54)c3)nc(-c3ccc4oc5ccccc5c4c3)n2)cc1 ; c1ccc(-c2ccc(-c3nc(-c4ccc5oc6ccccc6c5c4)nc(-c4cccc5oc6c(-c7ccccc7)cccc6c45)n3)cc2)cc1]

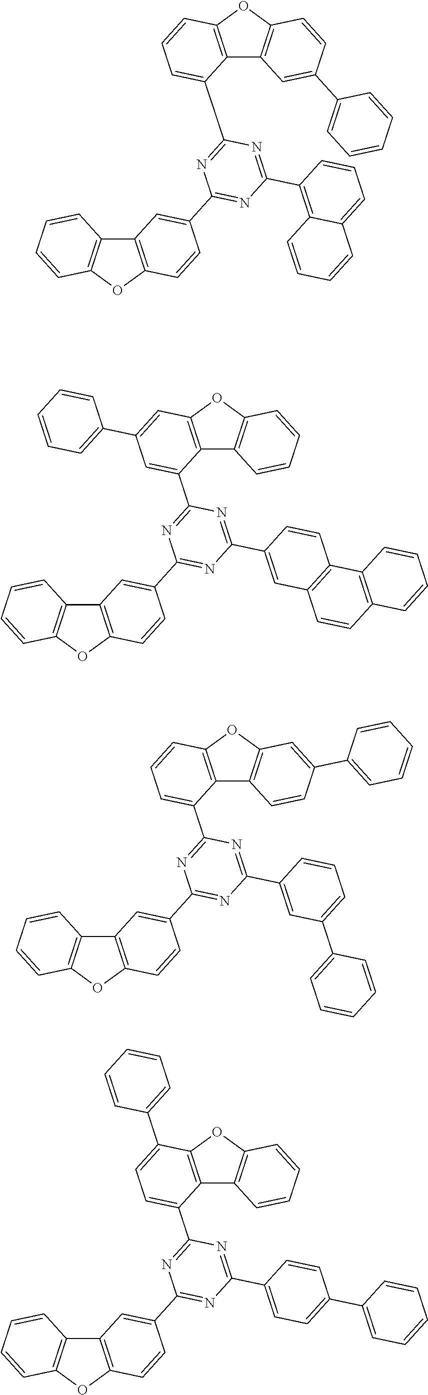 [SMILES: c1ccc(-c2ccc3oc4cccc(-c5nc(-c6ccc7oc8ccccc8c7c6)nc(-c6cccc7ccccc67)n5)c4c3c2)cc1 ; c1ccc(-c2cc(-c3nc(-c4ccc5c(ccc6ccccc65)c4)nc(-c4ccc5oc6ccccc6c5c4)n3)c3c(c2)oc2ccccc23)cc1 ; c1ccc(-c2cccc(-c3nc(-c4ccc5oc6ccccc6c5c4)nc(-c4cccc5oc6cc(-c7ccccc7)ccc6c45)n3)c2)cc1 ; c1ccc(-c2ccc(-c3nc(-c4ccc5oc6ccccc6c5c4)nc(-c4ccc(-c5ccccc5)c5oc6ccccc6c45)n3)cc2)cc1]

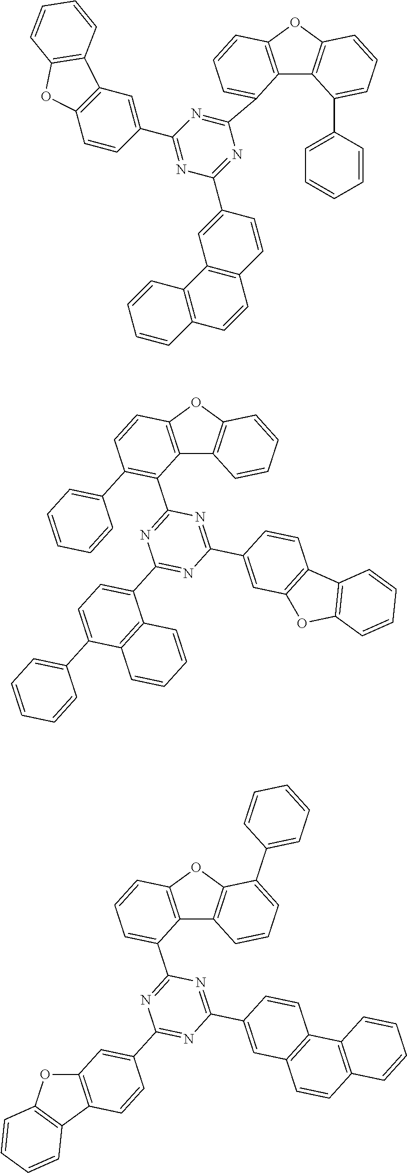 [SMILES: c1ccc(-c2cccc3oc4cccc(-c5nc(-c6ccc7ccc8ccccc8c7c6)nc(-c6ccc7oc8ccccc8c7c6)n5)c4c23)cc1 ; c1ccc(-c2ccc3oc4ccccc4c3c2-c2nc(-c3ccc4c(c3)oc3ccccc34)nc(-c3ccc(-c4ccccc4)c4ccccc34)n2)cc1 ; c1ccc(-c2cccc3c2oc2cccc(-c4nc(-c5ccc6c(ccc7ccccc76)c5)nc(-c5ccc6c(c5)oc5ccccc56)n4)c23)cc1]

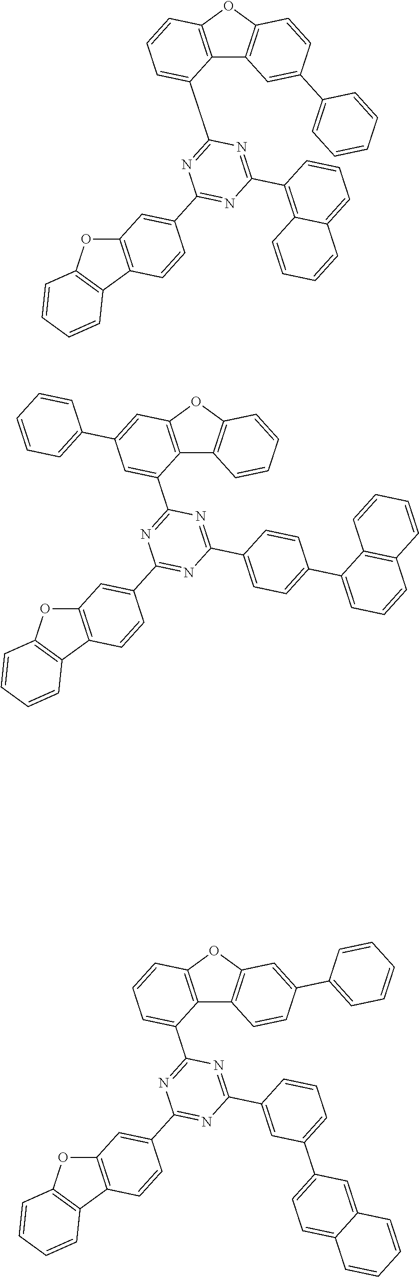 [SMILES: c1ccc(-c2ccc3oc4cccc(-c5nc(-c6ccc7c(c6)oc6ccccc67)nc(-c6cccc7ccccc67)n5)c4c3c2)cc1 ; c1ccc(-c2cc(-c3nc(-c4ccc(-c5cccc6ccccc56)cc4)nc(-c4ccc5c(c4)oc4ccccc45)n3)c3c(c2)oc2ccccc23)cc1 ; c1ccc(-c2ccc3c(c2)oc2cccc(-c4nc(-c5cccc(-c6ccc7ccccc7c6)c5)nc(-c5ccc6c(c5)oc5ccccc56)n4)c23)cc1]

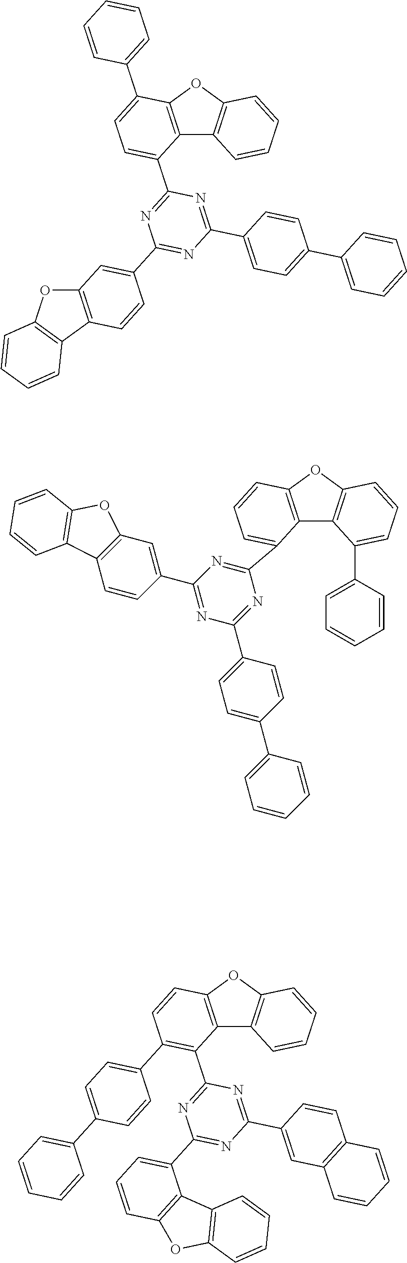 [SMILES: c1ccc(-c2ccc(-c3nc(-c4ccc5c(c4)oc4ccccc45)nc(-c4ccc(-c5ccccc5)c5oc6ccccc6c45)n3)cc2)cc1 ; c1ccc(-c2ccc(-c3nc(-c4ccc5c(c4)oc4ccccc45)nc(-c4cccc5oc6cccc(-c7ccccc7)c6c45)n3)cc2)cc1 ; c1ccc(-c2ccc(-c3ccc4oc5ccccc5c4c3-c3nc(-c4ccc5ccccc5c4)nc(-c4cccc5oc6ccccc6c45)n3)cc2)cc1]

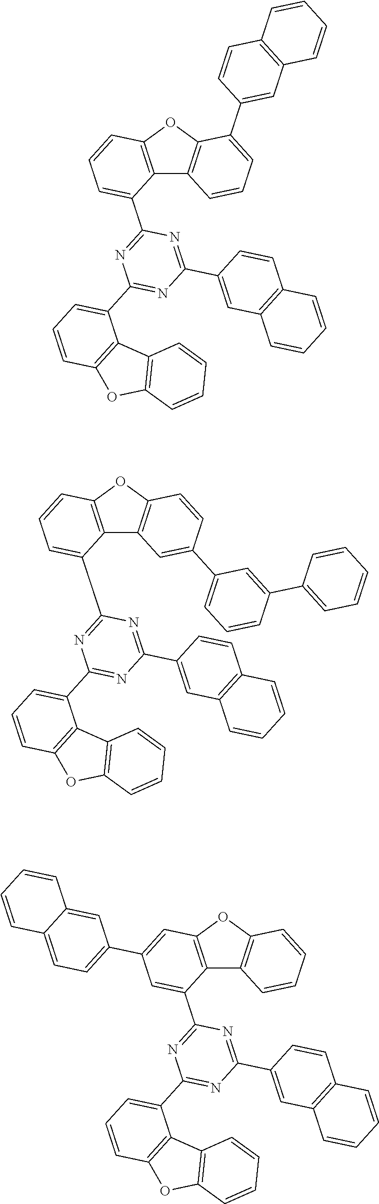 [SMILES: c1ccc2cc(-c3nc(-c4cccc5oc6ccccc6c45)nc(-c4cccc5oc6c(-c7ccc8ccccc8c7)cccc6c45)n3)ccc2c1 ; c1ccc(-c2cccc(-c3ccc4oc5cccc(-c6nc(-c7ccc8ccccc8c7)nc(-c7cccc8oc9ccccc9c78)n6)c5c4c3)c2)cc1 ; c1ccc2cc(-c3cc(-c4nc(-c5ccc6ccccc6c5)nc(-c5cccc6oc7ccccc7c56)n4)c4c(c3)oc3ccccc34)ccc2c1]

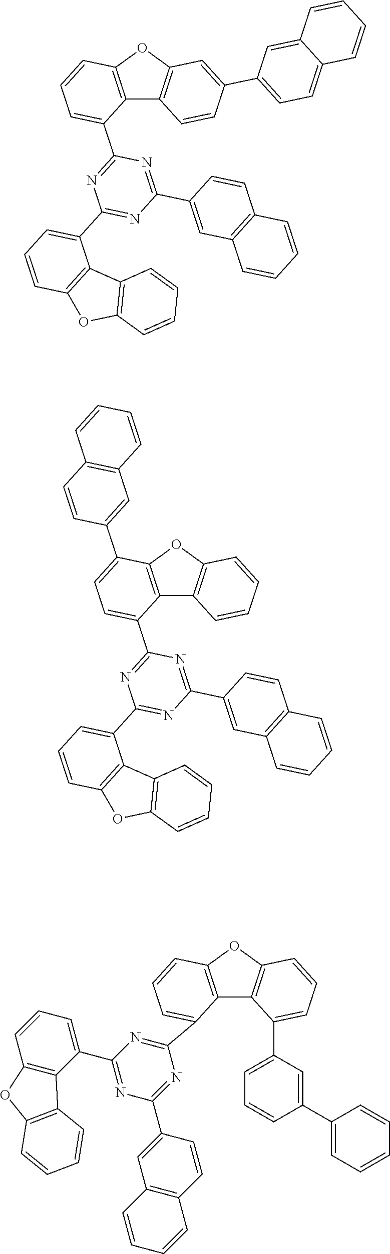 [SMILES: c1ccc2cc(-c3ccc4c(c3)oc3cccc(-c5nc(-c6ccc7ccccc7c6)nc(-c6cccc7oc8ccccc8c67)n5)c34)ccc2c1 ; c1ccc2cc(-c3nc(-c4cccc5oc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8oc9ccccc9c78)n6)c5c34)c2)cc1]

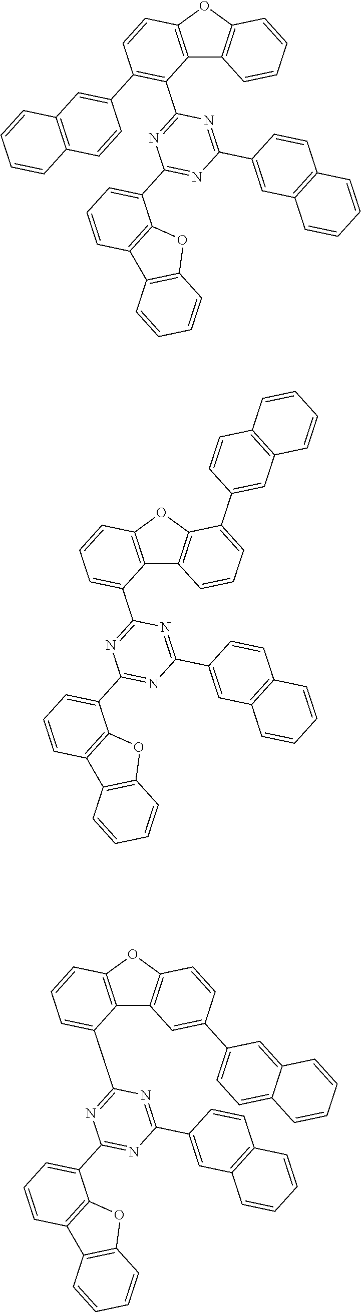 [SMILES: c1ccc2cc(-c3nc(-c4cccc5c4oc4ccccc45)nc(-c4c(-c5ccc6ccccc6c5)ccc5oc6ccccc6c45)n3)ccc2c1 ; c1ccc2cc(-c3nc(-c4cccc5c4oc4ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cccc8c7oc7ccccc78)n6)c5c4c3)ccc2c1]

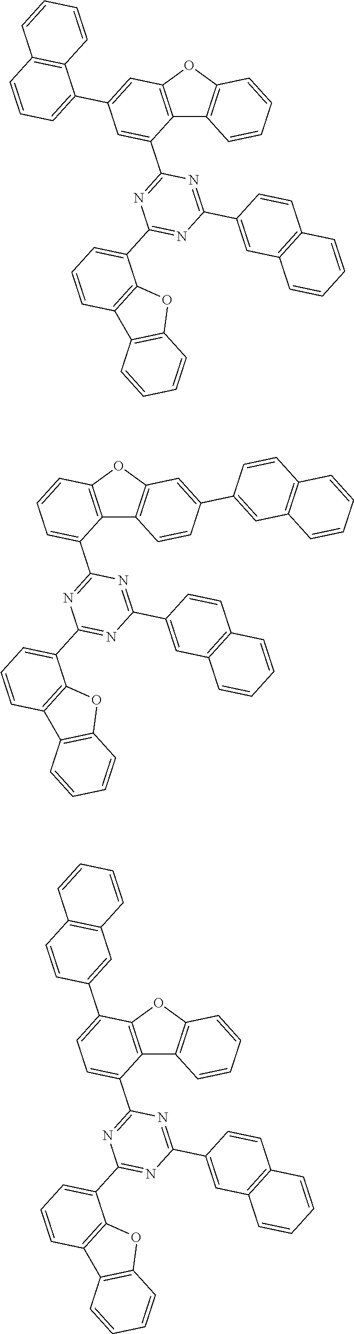 [SMILES: c1ccc2cc(-c3nc(-c4cccc5c4oc4ccccc45)nc(-c4cc(-c5cccc6ccccc56)cc5oc6ccccc6c45)n3)ccc2c1 ; c1ccc2cc(-c3ccc4c(c3)oc3cccc(-c5nc(-c6ccc7ccccc7c6)nc(-c6cccc7c6oc6ccccc67)n5)c34)ccc2c1 ; c1ccc2cc(-c3nc(-c4cccc5c4oc4ccccc45)nc(-c4ccc(-c5ccc6ccccc6c5)c5oc6ccccc6c45)n3)ccc2c1]

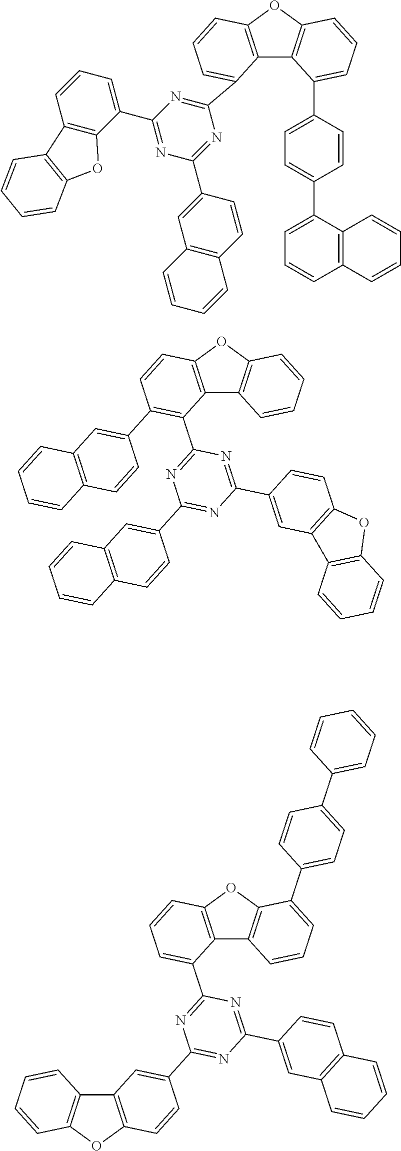

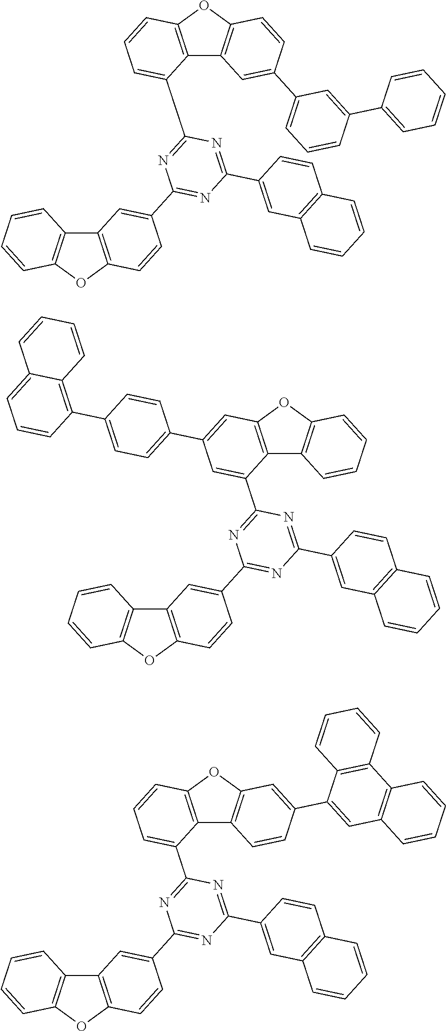

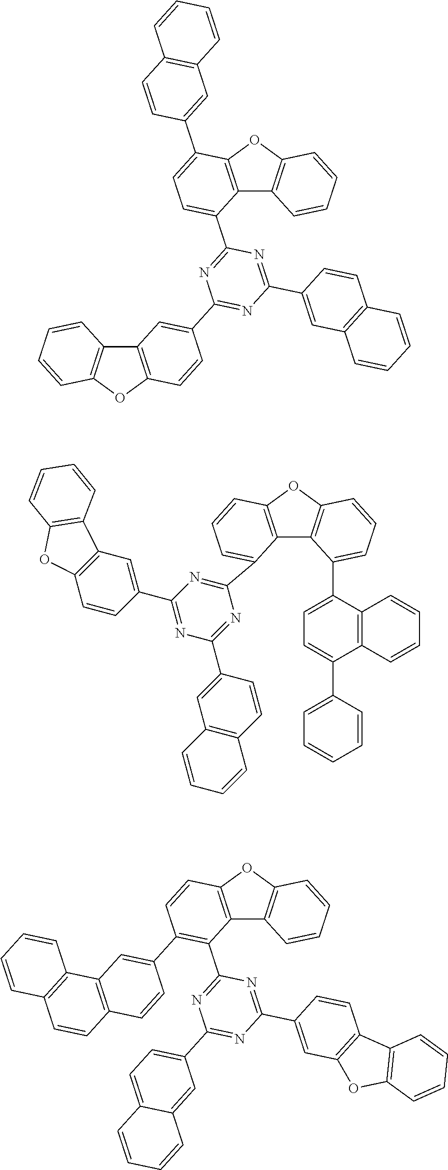 [SMILES: c1ccc2cc(-c3nc(-c4ccc5oc6ccccc6c5c4)nc(-c4ccc(-c5ccc6ccccc6c5)c5oc6ccccc6c45)n3)ccc2c1 ; c1ccc(-c2ccc(-c3cccc4oc5cccc(-c6nc(-c7ccc8ccccc8c7)nc(-c7ccc8oc9ccccc9c8c7)n6)c5c34)c3ccccc23)cc1 ; c1ccc2cc(-c3nc(-c4ccc5c(c4)oc4ccccc45)nc(-c4c(-c5ccc6ccc7ccccc7c6c5)ccc5oc6ccccc6c45)n3)ccc2c1]

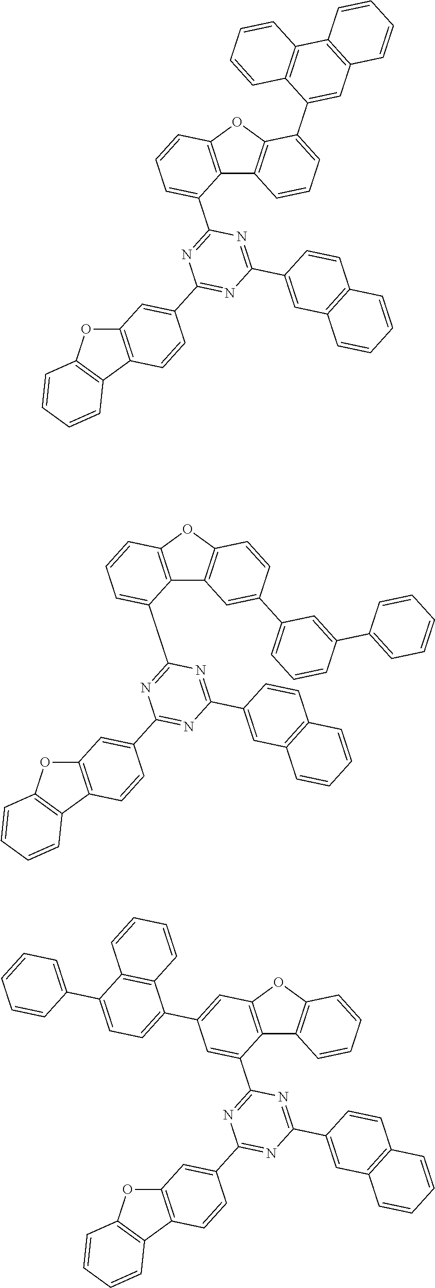 [SMILES: c1ccc2cc(-c3nc(-c4ccc5c(c4)oc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6nc(-c7ccc8ccccc8c7)nc(-c7ccc8c(c7)oc7ccccc78)n6)c5c4c3)c2)cc1 ; c1ccc(-c2ccc(-c3cc(-c4nc(-c5ccc6ccccc6c5)nc(-c5ccc6c(c5)oc5ccccc56)n4)c4c(c3)oc3ccccc34)c3ccccc23)cc1]

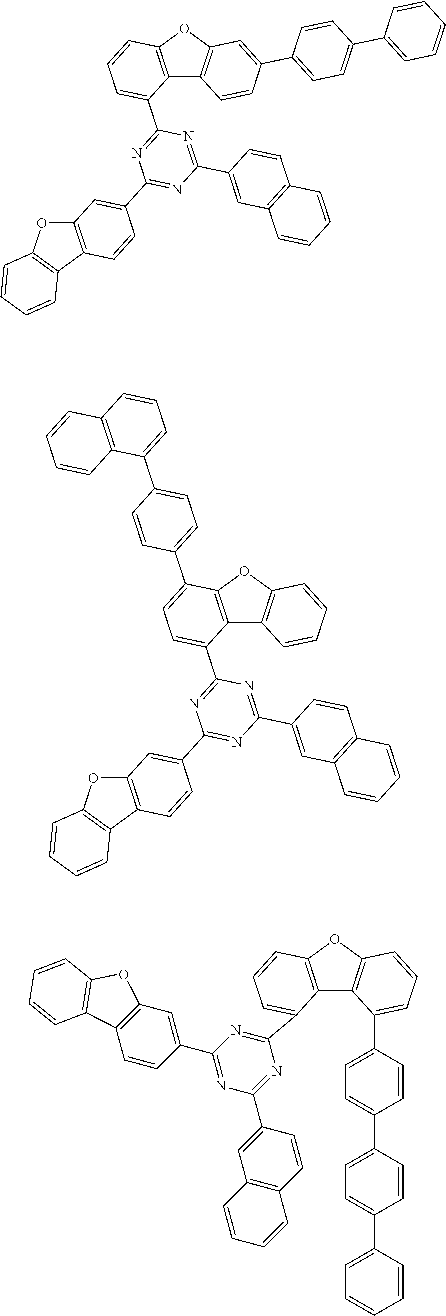 [SMILES: c1ccc(-c2ccc(-c3ccc4c(c3)oc3cccc(-c5nc(-c6ccc7ccccc7c6)nc(-c6ccc7c(c6)oc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1 ; c1ccc(-c2ccc(-c3ccc(-c4cccc5oc6cccc(-c7nc(-c8ccc9ccccc9c8)nc(-c8ccc9c(c8)oc8ccccc89)n7)c6c45)cc3)cc2)cc1]

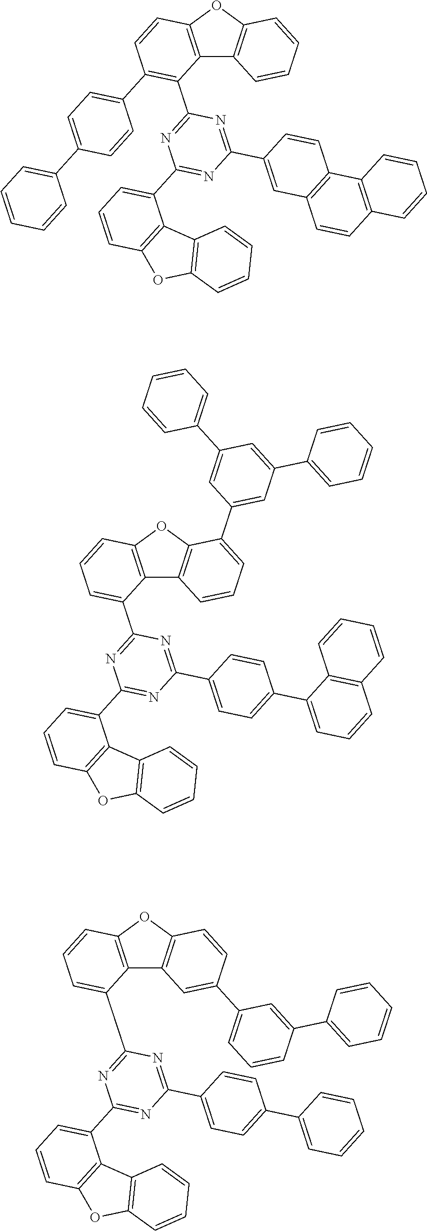 [SMILES: c1ccc(-c2ccc(-c3ccc4oc5ccccc5c4c3-c3nc(-c4ccc5c(ccc6ccccc65)c4)nc(-c4cccc5oc6ccccc6c45)n3)cc2)cc1 ; c1ccc(-c2cc(-c3ccccc3)cc(-c3cccc4c3oc3cccc(-c5nc(-c6ccc(-c7cccc8ccccc78)cc6)nc(-c6cccc7oc8ccccc8c67)n5)c34)c2)cc1 ; c1ccc(-c2ccc(-c3nc(-c4cccc5oc6ccccc6c45)nc(-c4cccc5oc6ccc(-c7cccc(-c8ccccc8)c7)cc6c45)n3)cc2)cc1]

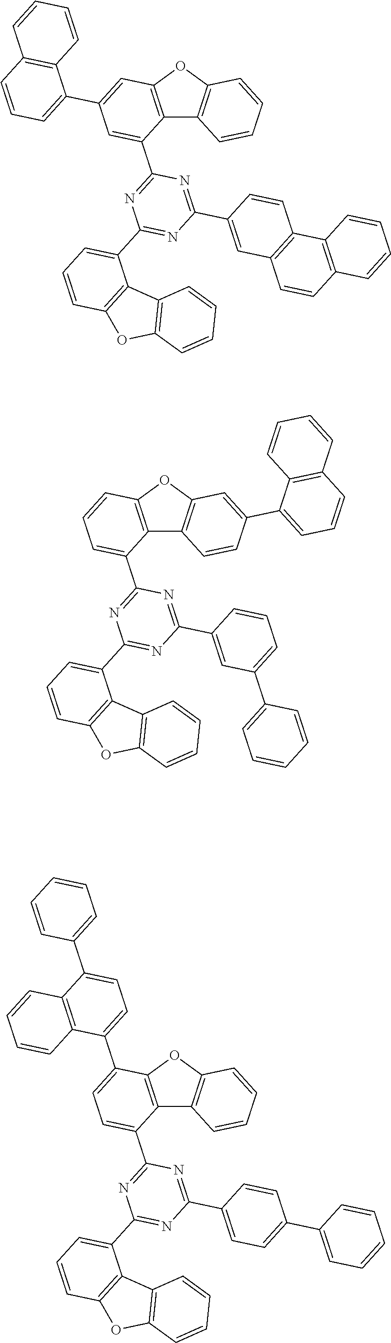 [SMILES: c1ccc2c(-c3cc(-c4nc(-c5ccc6c(ccc7ccccc76)c5)nc(-c5cccc6oc7ccccc7c56)n4)c4c(c3)oc3ccccc34)cccc2c1 ; c1ccc(-c2cccc(-c3nc(-c4cccc5oc6ccccc6c45)nc(-c4cccc5oc6cc(-c7cccc8ccccc78)ccc6c45)n3)c2)cc1 ; c1ccc(-c2ccc(-c3nc(-c4cccc5oc6ccccc6c45)nc(-c4ccc(-c5ccc(-c6ccccc6)c6ccccc56)c5oc6ccccc6c45)n3)cc2)cc1]

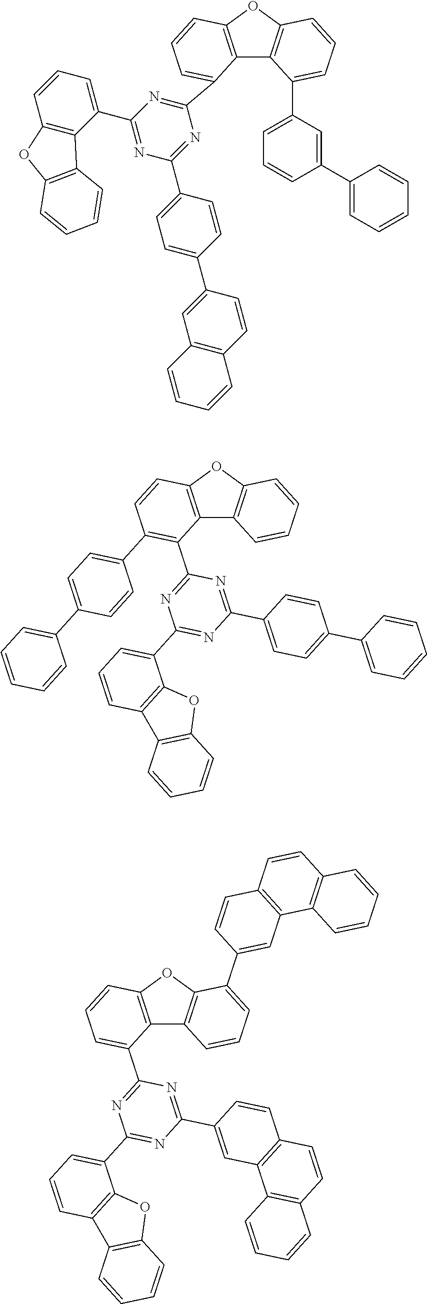 [SMILES: c1ccc(-c2cccc(-c3cccc4oc5cccc(-c6nc(-c7ccc(-c8ccc9ccccc9c8)cc7)nc(-c7cccc8oc9ccccc9c78)n6)c5c34)c2)cc1 ; c1ccc(-c2ccc(-c3nc(-c4cccc5c4oc4ccccc45)nc(-c4c(-c5ccc(-c6ccccc6)cc5)ccc5oc6ccccc6c45)n3)cc2)cc1 ; c1ccc2c(c1)ccc1ccc(-c3nc(-c4cccc5c4oc4ccccc45)nc(-c4cccc5oc6c(-c7ccc8ccc9ccccc9c8c7)cccc6c45)n3)cc12]

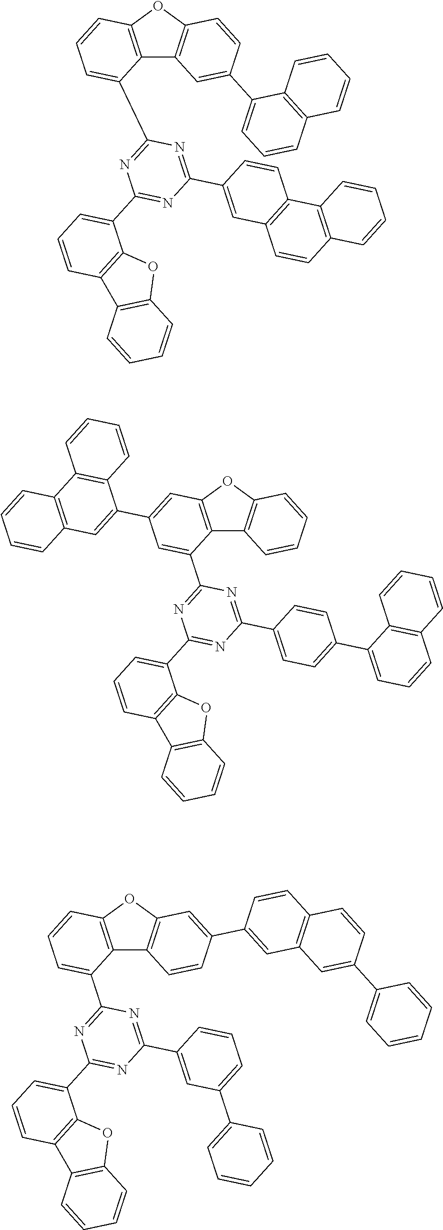 [SMILES: c1ccc2c(-c3ccc4oc5cccc(-c6nc(-c7ccc8c(ccc9ccccc98)c7)nc(-c7cccc8c7oc7ccccc78)n6)c5c4c3)cccc2c1 ; c1ccc2c(-c3ccc(-c4nc(-c5cccc6c5oc5ccccc56)nc(-c5cc(-c6cc7ccccc7c7ccccc67)cc6oc7ccccc7c56)n4)cc3)cccc2c1 ; c1ccc(-c2cccc(-c3nc(-c4cccc5c4oc4ccccc45)nc(-c4cccc5oc6cc(-c7ccc8ccc(-c9ccccc9)cc8c7)ccc6c45)n3)c2)cc1]

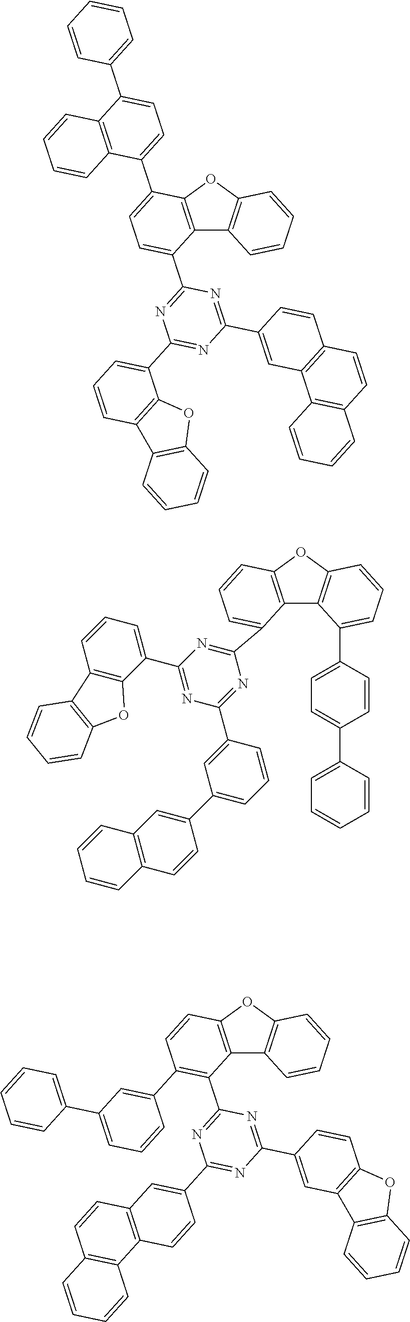 [SMILES: c1ccc(-c2ccc(-c3ccc(-c4nc(-c5ccc6ccc7ccccc7c6c5)nc(-c5cccc6c5oc5ccccc56)n4)c4c3oc3ccccc34)c3ccccc23)cc1 ; c1ccc(-c2ccc(-c3cccc4oc5cccc(-c6nc(-c7cccc(-c8ccc9ccccc9c8)c7)nc(-c7cccc8c7oc7ccccc78)n6)c5c34)cc2)cc1 ; c1ccc(-c2cccc(-c3ccc4oc5ccccc5c4c3-c3nc(-c4ccc5c(ccc6ccccc65)c4)nc(-c4ccc5oc6ccccc6c5c4)n3)c2)cc1]

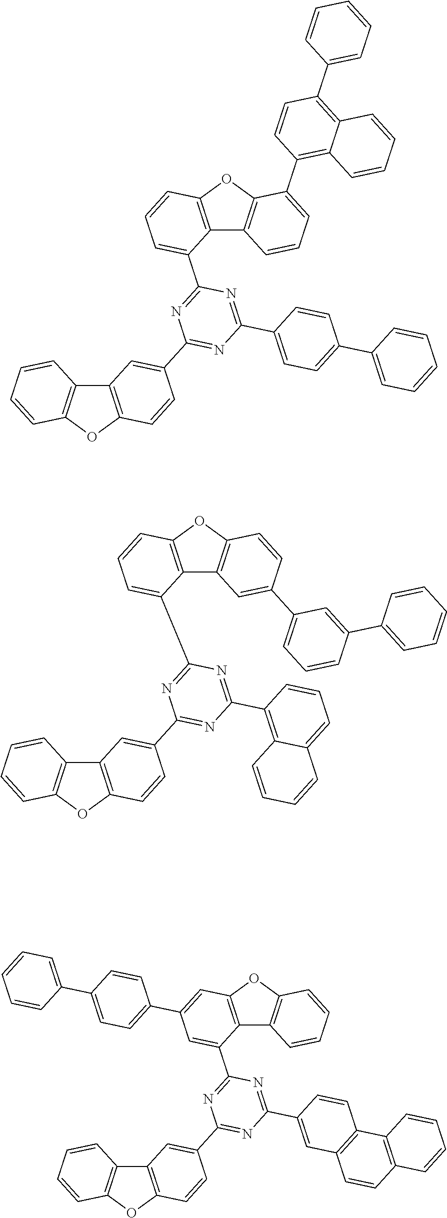 [SMILES: c1ccc(-c2ccc(-c3nc(-c4ccc5oc6ccccc6c5c4)nc(-c4cccc5oc6c(-c7ccc(-c8ccccc8)c8ccccc78)cccc6c45)n3)cc2)cc1 ; c1ccc(-c2cccc(-c3ccc4oc5cccc(-c6nc(-c7ccc8oc9ccccc9c8c7)nc(-c7cccc8ccccc78)n6)c5c4c3)c2)cc1 ; c1ccc(-c2ccc(-c3cc(-c4nc(-c5ccc6c(ccc7ccccc76)c5)nc(-c5ccc6oc7ccccc7c6c5)n4)c4c(c3)oc3ccccc34)cc2)cc1]

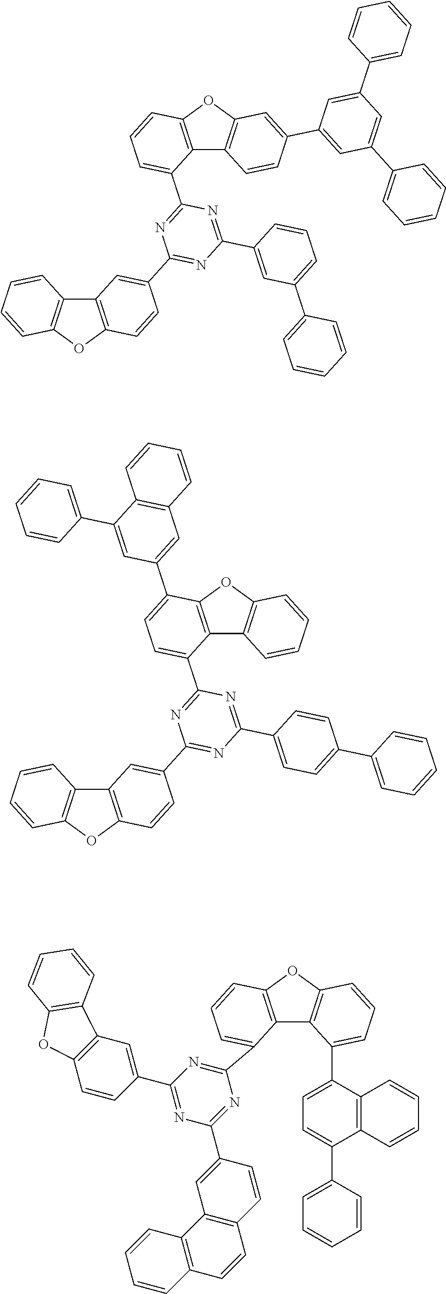 [SMILES: c1ccc(-c2cc(-c3ccccc3)cc(-c3ccc4c(c3)oc3cccc(-c5nc(-c6cccc(-c7ccccc7)c6)nc(-c6ccc7oc8ccccc8c7c6)n5)c34)c2)cc1 ; c1ccc(-c2ccc(-c3nc(-c4ccc5oc6ccccc6c5c4)nc(-c4ccc(-c5cc(-c6ccccc6)c6ccccc6c5)c5oc6ccccc6c45)n3)cc2)cc1 ; c1ccc(-c2ccc(-c3cccc4oc5cccc(-c6nc(-c7ccc8ccc9ccccc9c8c7)nc(-c7ccc8oc9ccccc9c8c7)n6)c5c34)c3ccccc23)cc1]

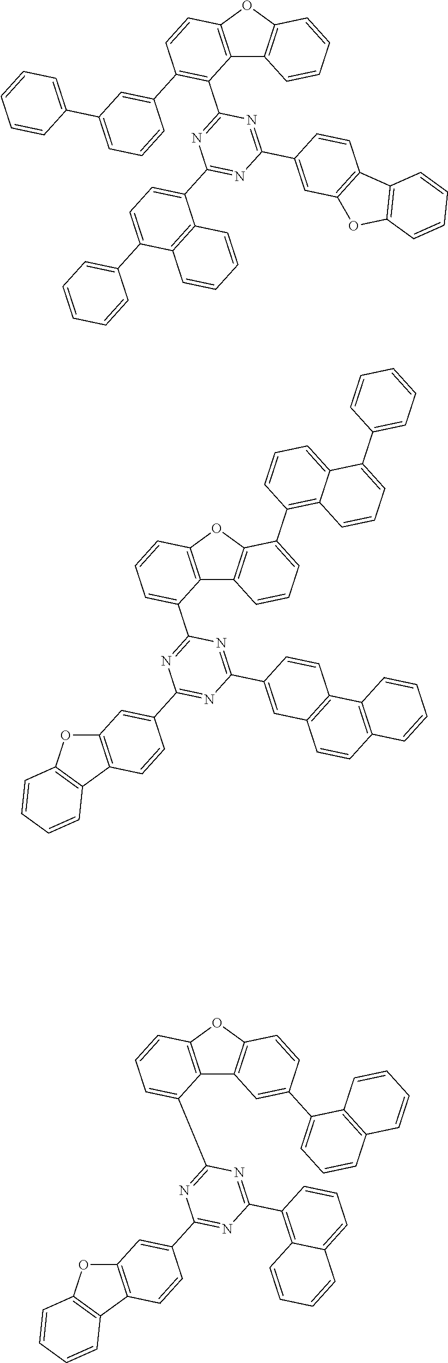 [SMILES: c1ccc(-c2cccc(-c3ccc4oc5ccccc5c4c3-c3nc(-c4ccc5c(c4)oc4ccccc45)nc(-c4ccc(-c5ccccc5)c5ccccc45)n3)c2)cc1 ; c1ccc(-c2cccc3c(-c4cccc5c4oc4cccc(-c6nc(-c7ccc8c(ccc9ccccc98)c7)nc(-c7ccc8c(c7)oc7ccccc78)n6)c45)cccc23)cc1 ; c1ccc2c(-c3ccc4oc5cccc(-c6nc(-c7ccc8c(c7)oc7ccccc78)nc(-c7cccc8ccccc78)n6)c5c4c3)cccc2c1]

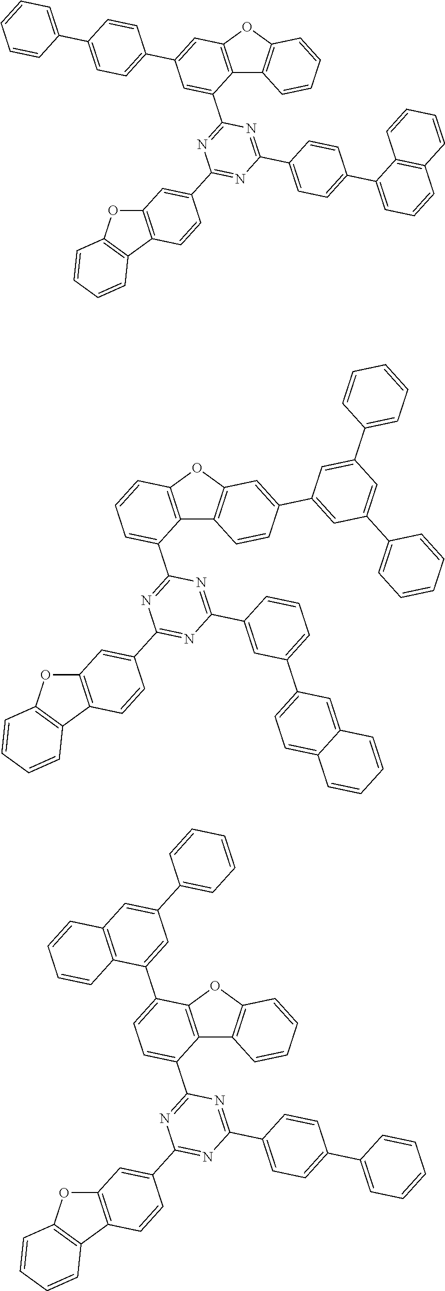 [SMILES: c1ccc(-c2ccc(-c3cc(-c4nc(-c5ccc(-c6cccc7ccccc67)cc5)nc(-c5ccc6c(c5)oc5ccccc56)n4)c4c(c3)oc3ccccc34)cc2)cc1 ; c1ccc(-c2cc(-c3ccccc3)cc(-c3ccc4c(c3)oc3cccc(-c5nc(-c6cccc(-c7ccc8ccccc8c7)c6)nc(-c6ccc7c(c6)oc6ccccc67)n5)c34)c2)cc1 ; c1ccc(-c2ccc(-c3nc(-c4ccc5c(c4)oc4ccccc45)nc(-c4ccc(-c5cc(-c6ccccc6)cc6ccccc56)c5oc6ccccc6c45)n3)cc2)cc1]

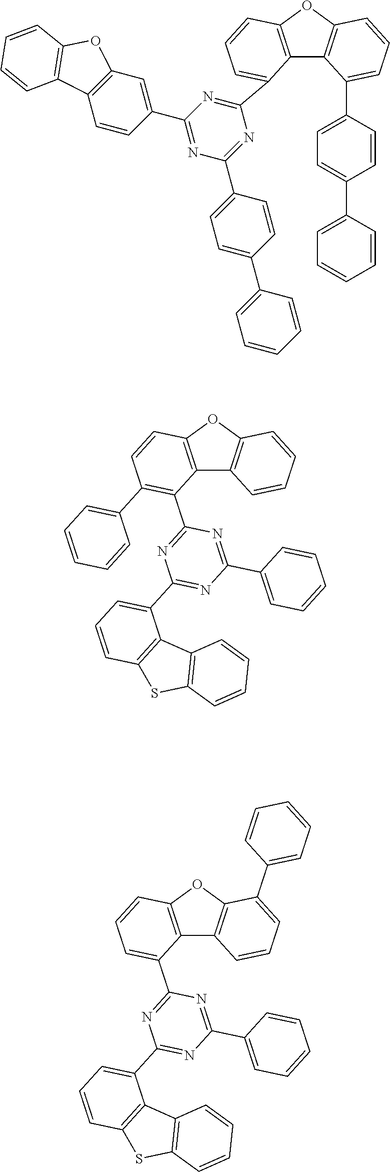 [SMILES: c1ccc(-c2ccc(-c3nc(-c4ccc5c(c4)oc4ccccc45)nc(-c4cccc5oc6cccc(-c7ccc(-c8ccccc8)cc7)c6c45)n3)cc2)cc1 ; c1ccc(-c2nc(-c3c(-c4ccccc4)ccc4oc5ccccc5c34)nc(-c3cccc4sc5ccccc5c34)n2)cc1 ; c1ccc(-c2nc(-c3cccc4oc5c(-c6ccccc6)cccc5c34)nc(-c3cccc4sc5ccccc5c34)n2)cc1]

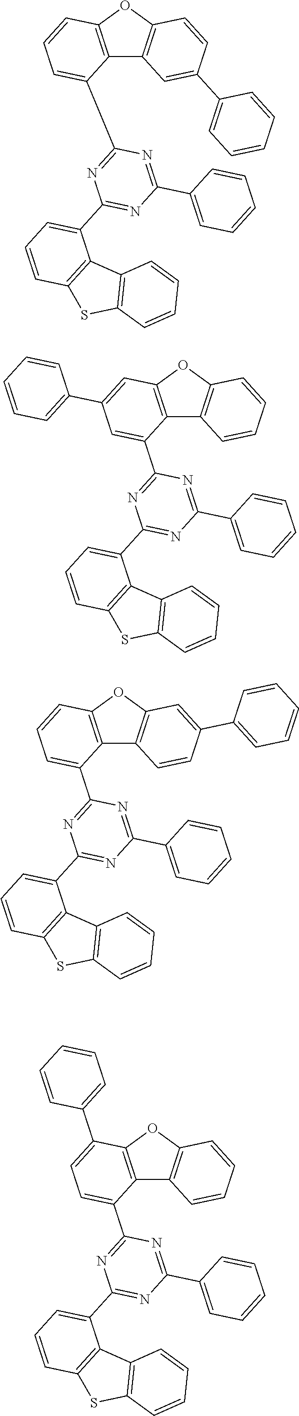 [SMILES: c1ccc(-c2ccc3oc4cccc(-c5nc(-c6ccccc6)nc(-c6cccc7sc8ccccc8c67)n5)c4c3c2)cc1 ; c1ccc(-c2cc(-c3nc(-c4ccccc4)nc(-c4cccc5sc6ccccc6c45)n3)c3c(c2)oc2ccccc23)cc1 ; c1ccc(-c2ccc3c(c2)oc2cccc(-c4nc(-c5ccccc5)nc(-c5cccc6sc7ccccc7c56)n4)c23)cc1 ; c1ccc(-c2nc(-c3cccc4sc5ccccc5c34)nc(-c3ccc(-c4ccccc4)c4oc5ccccc5c34)n2)cc1]

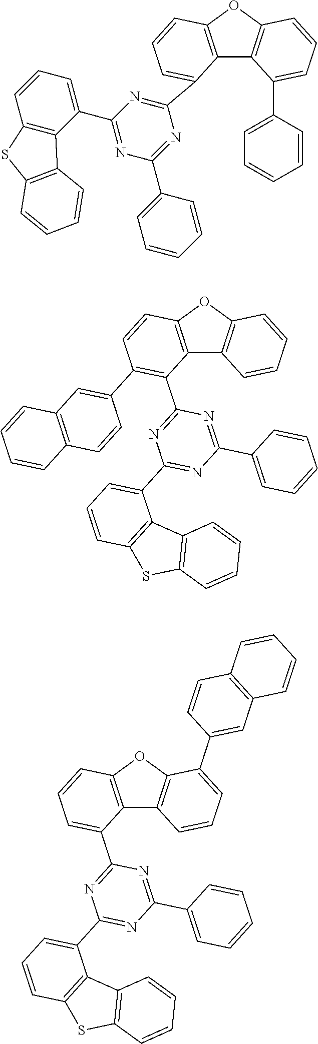 [SMILES: c1ccc(-c2nc(-c3cccc4sc5ccccc5c34)nc(-c3cccc4oc5cccc(-c6ccccc6)c5c34)n2)cc1 ; c1ccc(-c2nc(-c3c(-c4ccc5ccccc5c4)ccc4oc5ccccc5c34)nc(-c3cccc4sc5ccccc5c34)n2)cc1 ; c1ccc(-c2nc(-c3cccc4oc5c(-c6ccc7ccccc7c6)cccc5c34)nc(-c3cccc4sc5ccccc5c34)n2)cc1]

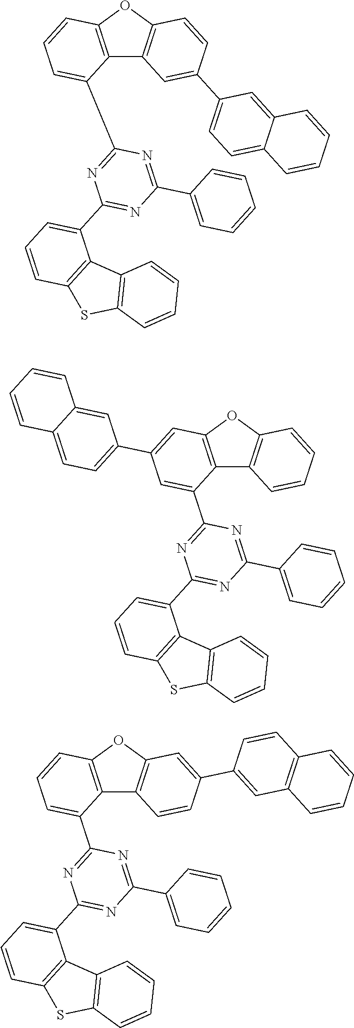

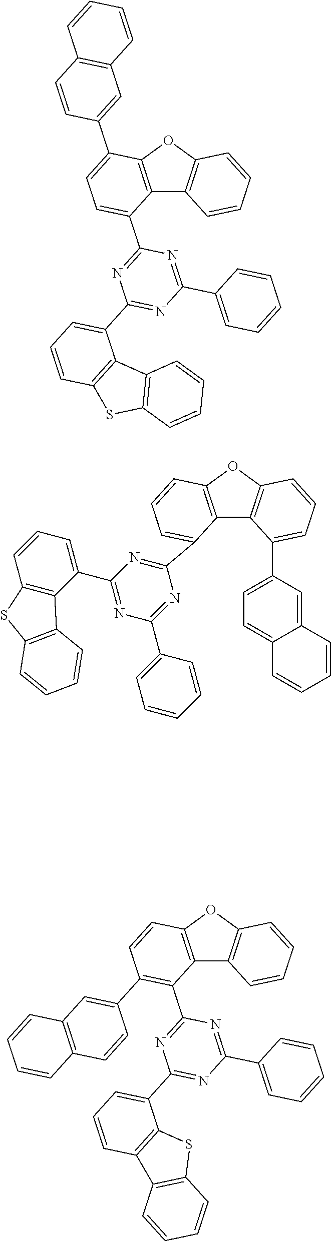 [SMILES: c1ccc(-c2nc(-c3cccc4sc5ccccc5c34)nc(-c3ccc(-c4ccc5ccccc5c4)c4oc5ccccc5c34)n2)cc1 ; c1ccc(-c2nc(-c3cccc4sc5ccccc5c34)nc(-c3cccc4oc5cccc(-c6ccc7ccccc7c6)c5c34)n2)cc1 ; c1ccc(-c2nc(-c3cccc4c3sc3ccccc34)nc(-c3c(-c4ccc5ccccc5c4)ccc4oc5ccccc5c34)n2)cc1]

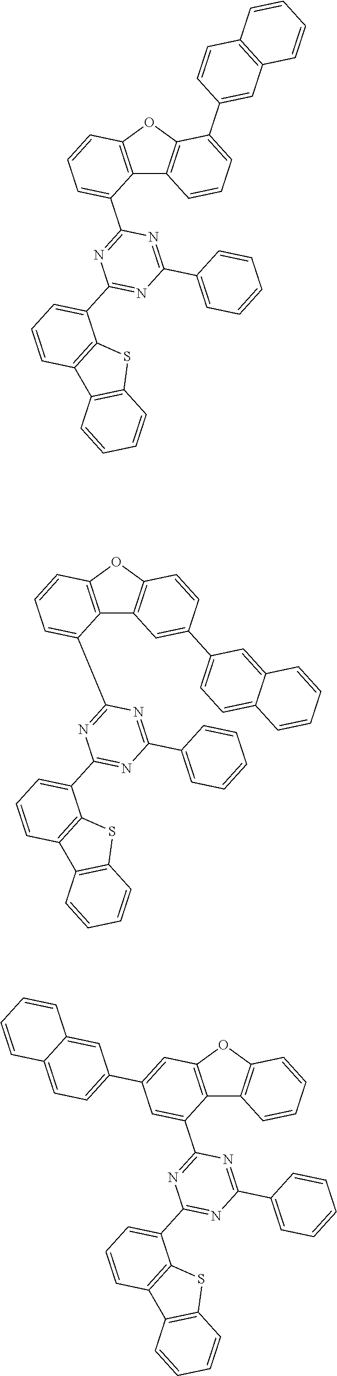 [SMILES: c1ccc(-c2nc(-c3cccc4c3sc3ccccc34)nc(-c3cccc4oc5c(-c6ccc7ccccc7c6)cccc5c34)n2)cc1 ; c1ccc(-c2nc(-c3cccc4c3sc3ccccc34)nc(-c3cccc4oc5ccc(-c6ccc7ccccc7c6)cc5c34)n2)cc1 ; c1ccc(-c2nc(-c3cccc4c3sc3ccccc34)nc(-c3cc(-c4ccc5ccccc5c4)cc4oc5ccccc5c34)n2)cc1]

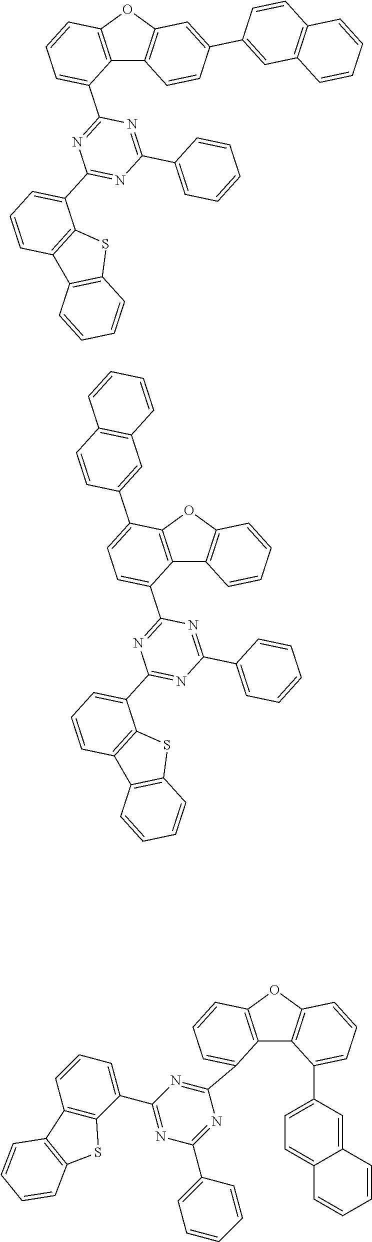

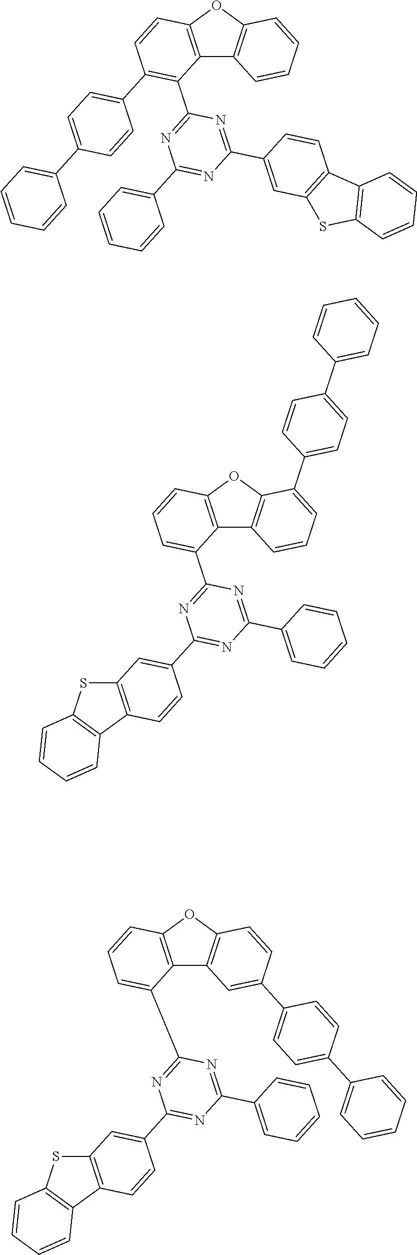 [SMILES: c1ccc(-c2ccc(-c3ccc4oc5ccccc5c4c3-c3nc(-c4ccccc4)nc(-c4ccc5c(c4)sc4ccccc45)n3)cc2)cc1 ; c1ccc(-c2ccc(-c3cccc4c3oc3cccc(-c5nc(-c6ccccc6)nc(-c6ccc7c(c6)sc6ccccc67)n5)c34)cc2)cc1 ; c1ccc(-c2ccc(-c3ccc4oc5cccc(-c6nc(-c7ccccc7)nc(-c7ccc8c(c7)sc7ccccc78)n6)c5c4c3)cc2)cc1]

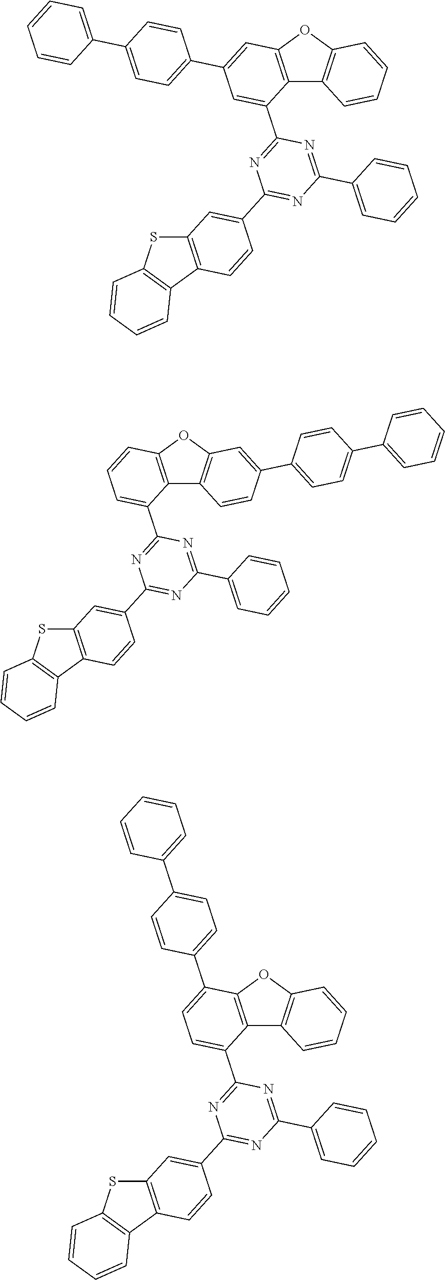 [SMILES: c1ccc(-c2ccc(-c3cc(-c4nc(-c5ccccc5)nc(-c5ccc6c(c5)sc5ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc7c(c6)sc6ccccc67)n5)c34)cc2)cc1 ; c1ccc(-c2ccc(-c3ccc(-c4nc(-c5ccccc5)nc(-c5ccc6c(c5)sc5ccccc56)n4)c4c3oc3ccccc34)cc2)cc1]

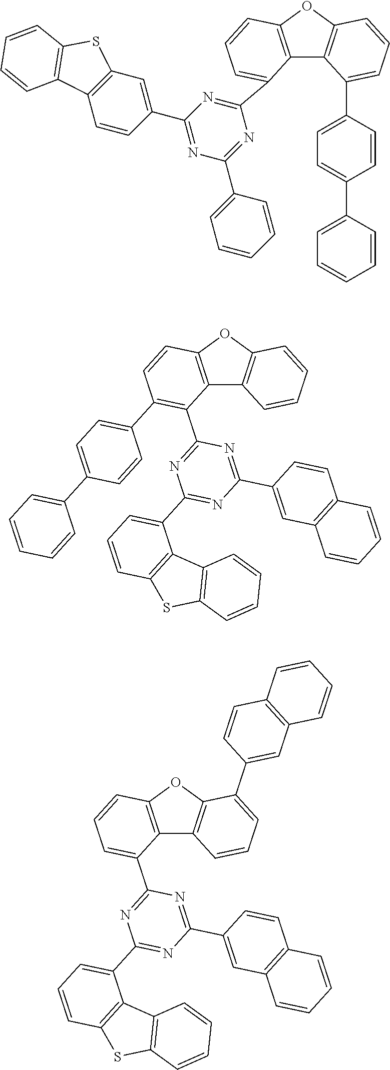 [SMILES: c1ccc(-c2ccc(-c3cccc4oc5cccc(-c6nc(-c7ccccc7)nc(-c7ccc8c(c7)sc7ccccc78)n6)c5c34)cc2)cc1 ; c1ccc(-c2ccc(-c3ccc4oc5ccccc5c4c3-c3nc(-c4ccc5ccccc5c4)nc(-c4cccc5sc6ccccc6c45)n3)cc2)cc1 ; c1ccc2cc(-c3nc(-c4cccc5oc6c(-c7ccc8ccccc8c7)cccc6c45)nc(-c4cccc5sc6ccccc6c45)n3)ccc2c1]

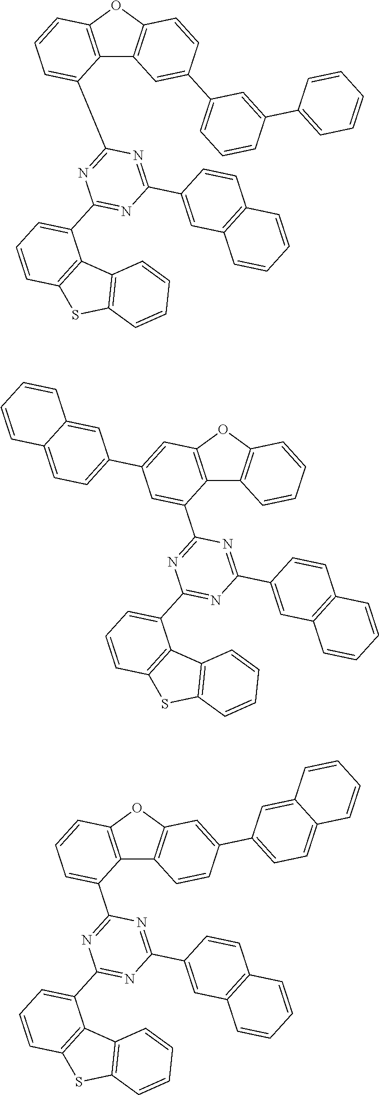 [SMILES: c1ccc(-c2cccc(-c3ccc4oc5cccc(-c6nc(-c7ccc8ccccc8c7)nc(-c7cccc8sc9ccccc9c78)n6)c5c4c3)c2)cc1 ; c1ccc2cc(-c3cc(-c4nc(-c5ccc6ccccc6c5)nc(-c5cccc6sc7ccccc7c56)n4)c4c(c3)oc3ccccc34)ccc2c1 ; c1ccc2cc(-c3ccc4c(c3)oc3cccc(-c5nc(-c6ccc7ccccc7c6)nc(-c6cccc7sc8ccccc8c67)n5)c34)ccc2c1]

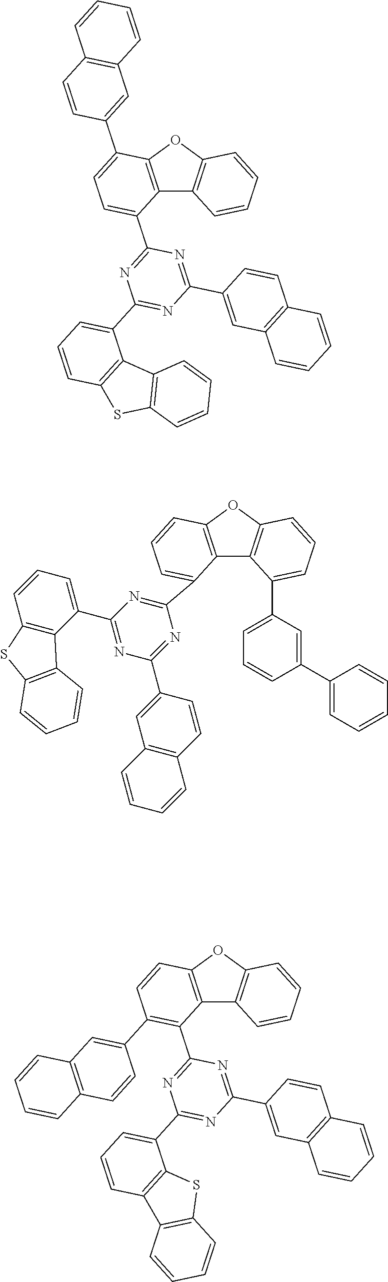 [SMILES: c1ccc2cc(-c3nc(-c4cccc5sc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8sc9ccccc9c78)n6)c5c34)c2)cc1 ; c1ccc2cc(-c3nc(-c4cccc5c4sc4ccccc45)nc(-c4c(-c5ccc6ccccc6c5)ccc5oc6ccccc6c45)n3)ccc2c1]

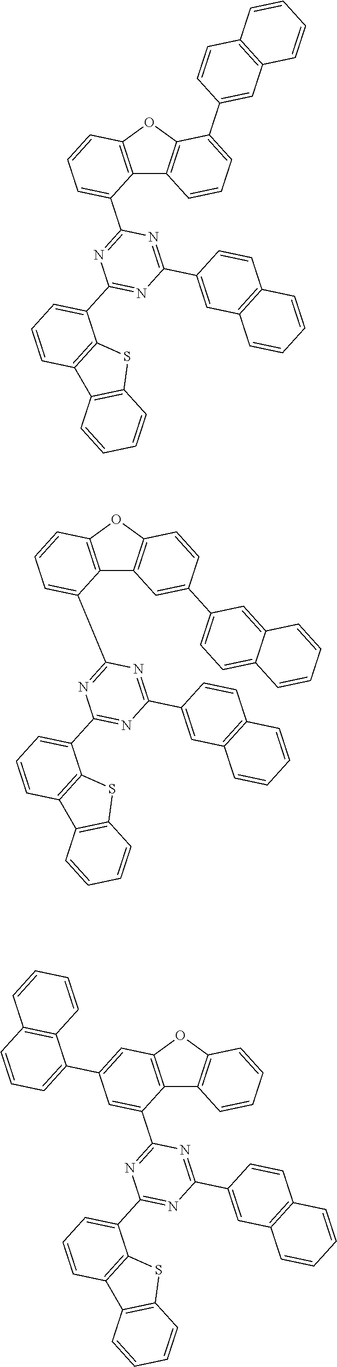 [SMILES: c1ccc2cc(-c3nc(-c4cccc5c4sc4ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cccc8c7sc7ccccc78)n6)c5c4c3)ccc2c1 ; c1ccc2cc(-c3nc(-c4cccc5c4sc4ccccc45)nc(-c4cc(-c5cccc6ccccc56)cc5oc6ccccc6c45)n3)ccc2c1]

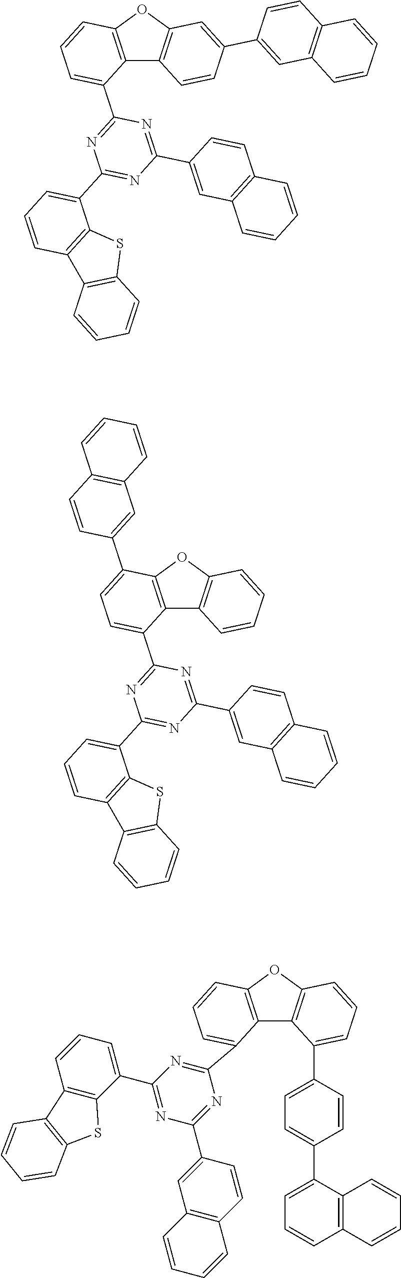 [SMILES: c1ccc2cc(-c3ccc4c(c3)oc3cccc(-c5nc(-c6ccc7ccccc7c6)nc(-c6cccc7c6sc6ccccc67)n5)c34)ccc2c1 ; c1ccc2cc(-c3nc(-c4cccc5c4sc4ccccc45)nc(-c4ccc(-c5ccc6ccccc6c5)c5oc6ccccc6c45)n3)ccc2c1 ; c1ccc2cc(-c3nc(-c4cccc5c4sc4ccccc45)nc(-c4cccc5oc6cccc(-c7ccc(-c8cccc9ccccc89)cc7)c6c45)n3)ccc2c1]

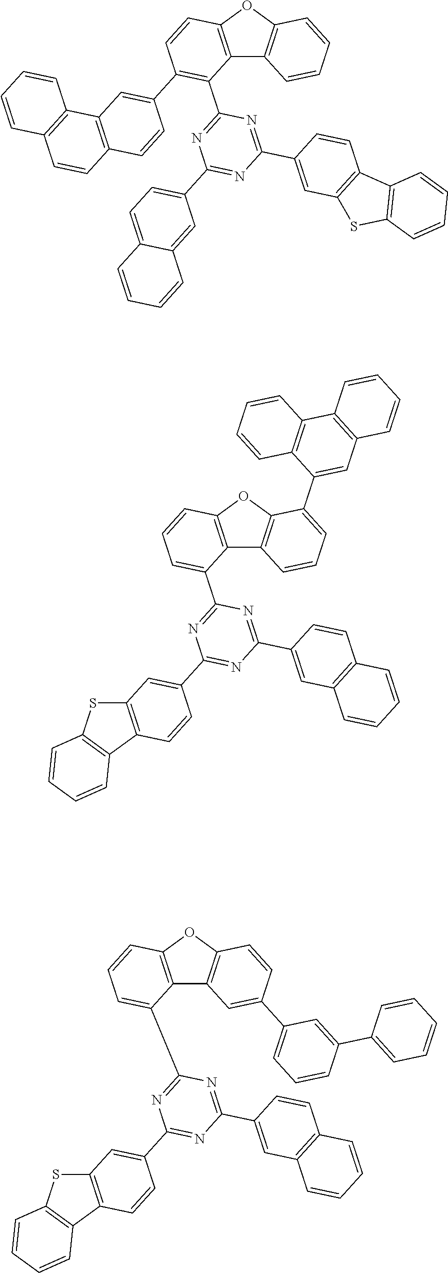 [SMILES: c1ccc2cc(-c3nc(-c4ccc5c(c4)sc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)sc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6nc(-c7ccc8ccccc8c7)nc(-c7ccc8c(c7)sc7ccccc78)n6)c5c4c3)c2)cc1]

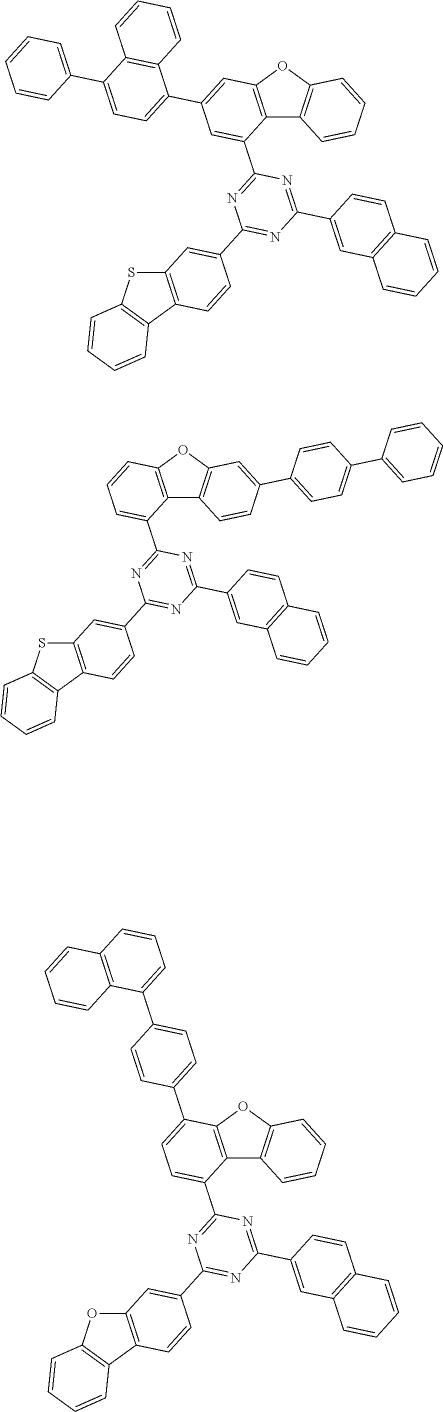 [SMILES: c1ccc(-c2ccc(-c3cc(-c4nc(-c5ccc6ccccc6c5)nc(-c5ccc6c(c5)sc5ccccc56)n4)c4c(c3)oc3ccccc34)c3ccccc23)cc1 ; c1ccc(-c2ccc(-c3ccc4c(c3)oc3cccc(-c5nc(-c6ccc7ccccc7c6)nc(-c6ccc7c(c6)sc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1]

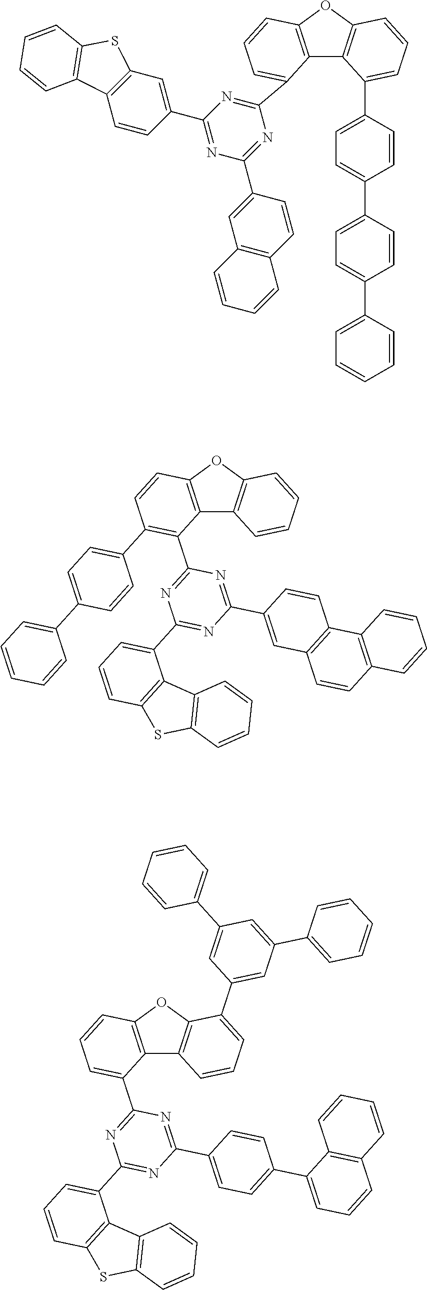 [SMILES: c1ccc(-c2ccc(-c3ccc(-c4cccc5oc6cccc(-c7nc(-c8ccc9ccccc9c8)nc(-c8ccc9c(c8)sc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5sc6ccccc6c45)n3)cc2)cc1 ; c1ccc(-c2cc(-c3ccccc3)cc(-c3cccc4c3oc3cccc(-c5nc(-c6ccc(-c7cccc8ccccc78)cc6)nc(-c6cccc7sc8ccccc8c67)n5)c34)c2)cc1]

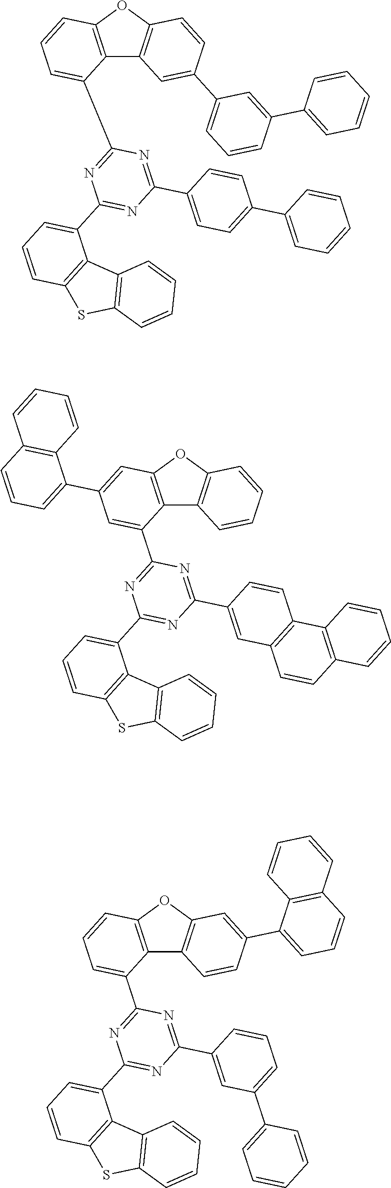 [SMILES: c1ccc(-c2ccc(-c3nc(-c4cccc5oc6ccc(-c7cccc(-c8ccccc8)c7)cc6c45)nc(-c4cccc5sc6ccccc6c45)n3)cc2)cc1 ; c1ccc2c(-c3cc(-c4nc(-c5ccc6c(ccc7ccccc76)c5)nc(-c5cccc6sc7ccccc7c56)n4)c4c(c3)oc3ccccc34)cccc2c1 ; c1ccc(-c2cccc(-c3nc(-c4cccc5oc6cc(-c7cccc8ccccc78)ccc6c45)nc(-c4cccc5sc6ccccc6c45)n3)c2)cc1]

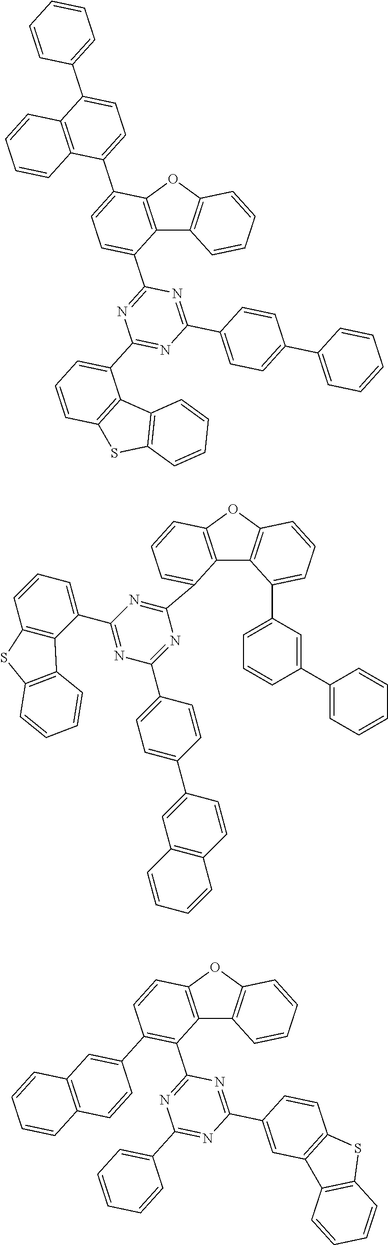 [SMILES: c1ccc(-c2ccc(-c3nc(-c4cccc5sc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8sc9ccccc9c78)n6)c5c34)c2)cc1 ; c1ccc(-c2nc(-c3ccc4sc5ccccc5c4c3)nc(-c3c(-c4ccc5ccccc5c4)ccc4oc5ccccc5c34)n2)cc1]

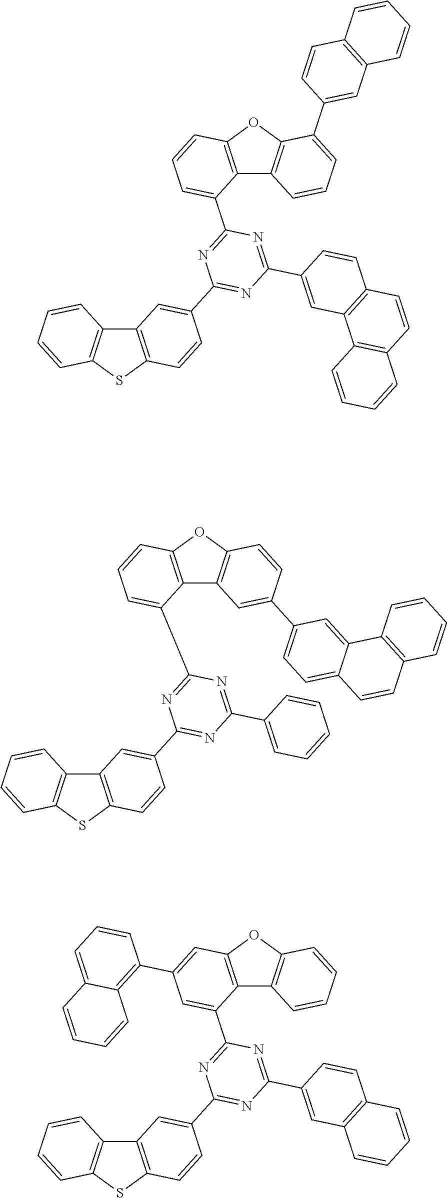 [SMILES: c1ccc2cc(-c3cccc4c3oc3cccc(-c5nc(-c6ccc7ccc8ccccc8c7c6)nc(-c6ccc7sc8ccccc8c7c6)n5)c34)ccc2c1 ; c1ccc(-c2nc(-c3ccc4sc5ccccc5c4c3)nc(-c3cccc4oc5ccc(-c6ccc7ccc8ccccc8c7c6)cc5c34)n2)cc1 ; c1ccc2cc(-c3nc(-c4ccc5sc6ccccc6c5c4)nc(-c4cc(-c5cccc6ccccc56)cc5oc6ccccc6c45)n3)ccc2c1]

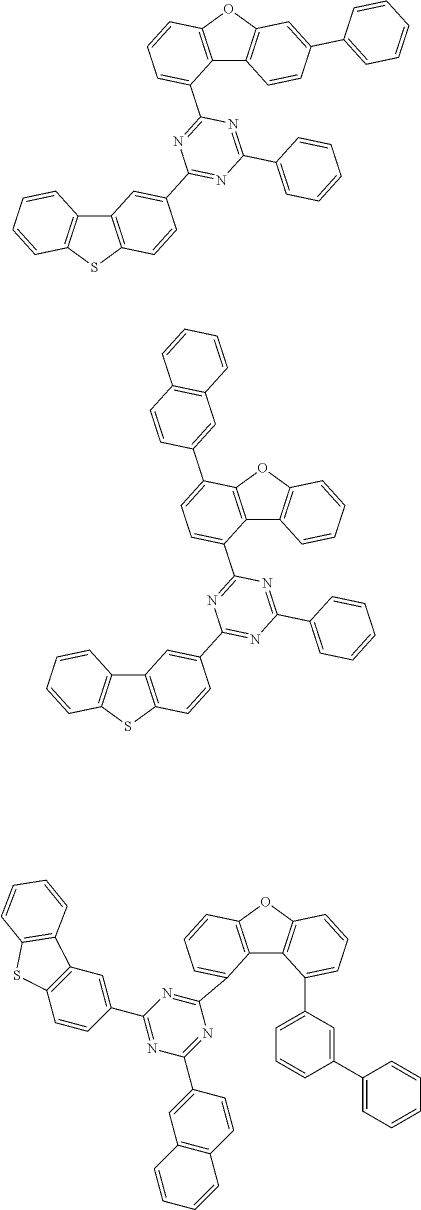 [SMILES: c1ccc(-c2ccc3c(c2)oc2cccc(-c4nc(-c5ccccc5)nc(-c5ccc6sc7ccccc7c6c5)n4)c23)cc1 ; c1ccc(-c2nc(-c3ccc4sc5ccccc5c4c3)nc(-c3ccc(-c4ccc5ccccc5c4)c4oc5ccccc5c34)n2)cc1 ; c1ccc(-c2cccc(-c3cccc4oc5cccc(-c6nc(-c7ccc8ccccc8c7)nc(-c7ccc8sc9ccccc9c8c7)n6)c5c34)c2)cc1]

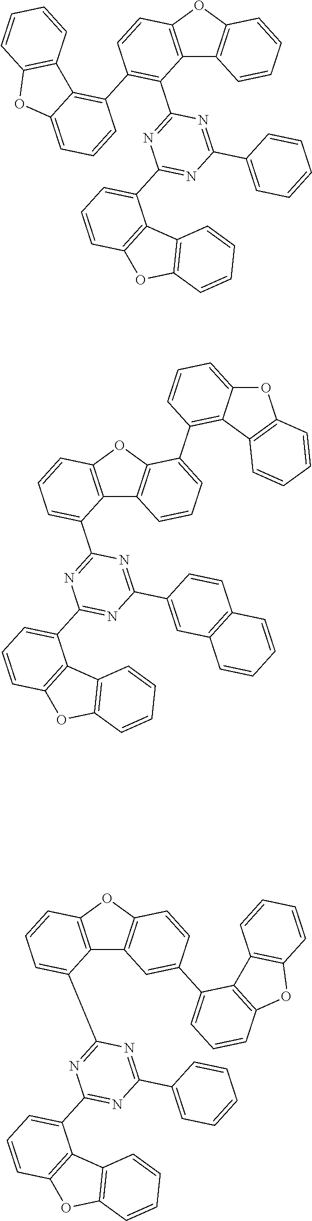 [SMILES: c1ccc(-c2nc(-c3cccc4oc5ccccc5c34)nc(-c3c(-c4cccc5oc6ccccc6c45)ccc4oc5ccccc5c34)n2)cc1 ; c1ccc2cc(-c3nc(-c4cccc5oc6ccccc6c45)nc(-c4cccc5oc6c(-c7cccc8oc9ccccc9c78)cccc6c45)n3)ccc2c1 ; c1ccc(-c2nc(-c3cccc4oc5ccccc5c34)nc(-c3cccc4oc5ccc(-c6cccc7oc8ccccc8c67)cc5c34)n2)cc1]

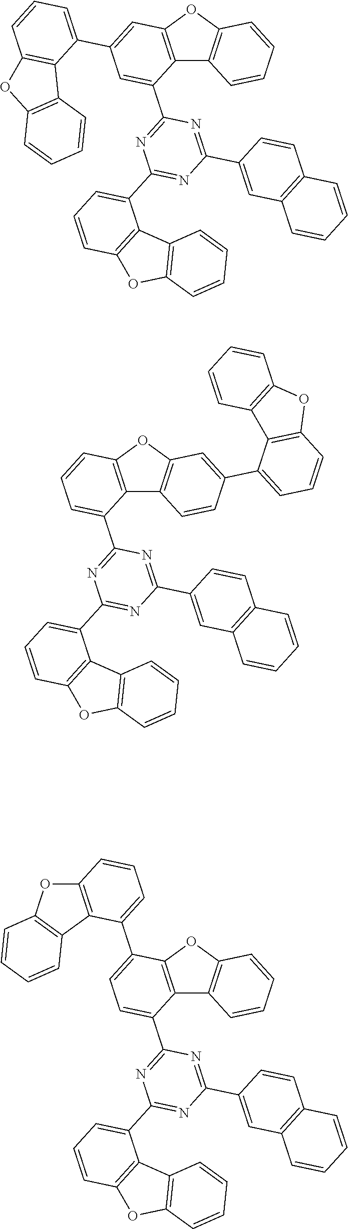 [SMILES: c1ccc2cc(-c3nc(-c4cccc5oc6ccccc6c45)nc(-c4cc(-c5cccc6oc7ccccc7c56)cc5oc6ccccc6c45)n3)ccc2c1 ; c1ccc2cc(-c3nc(-c4cccc5oc6ccccc6c45)nc(-c4cccc5oc6cc(-c7cccc8oc9ccccc9c78)ccc6c45)n3)ccc2c1 ; c1ccc2cc(-c3nc(-c4cccc5oc6ccccc6c45)nc(-c4ccc(-c5cccc6oc7ccccc7c56)c5oc6ccccc6c45)n3)ccc2c1]

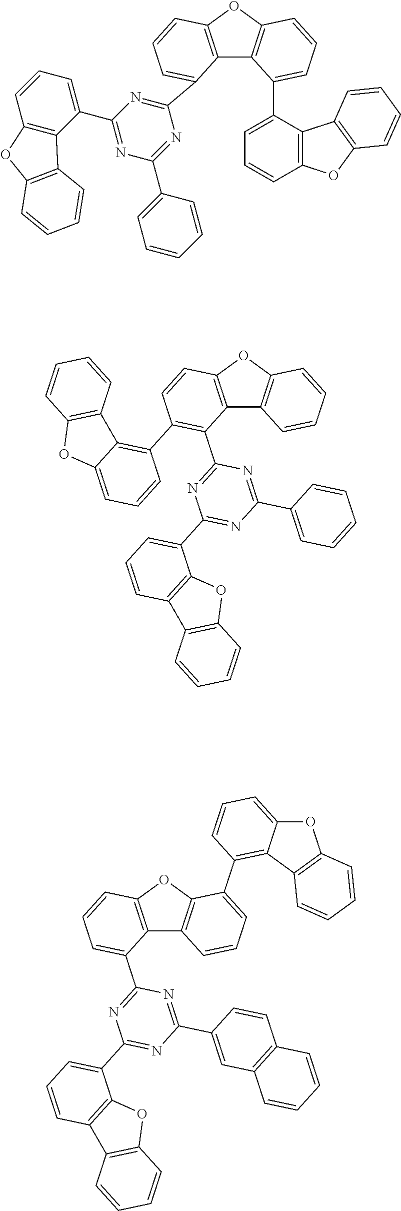 [SMILES: c1ccc(-c2nc(-c3cccc4oc5ccccc5c34)nc(-c3cccc4oc5cccc(-c6cccc7oc8ccccc8c67)c5c34)n2)cc1 ; c1ccc(-c2nc(-c3cccc4c3oc3ccccc34)nc(-c3c(-c4cccc5oc6ccccc6c45)ccc4oc5ccccc5c34)n2)cc1 ; c1ccc2cc(-c3nc(-c4cccc5c4oc4ccccc45)nc(-c4cccc5oc6c(-c7cccc8oc9ccccc9c78)cccc6c45)n3)ccc2c1]

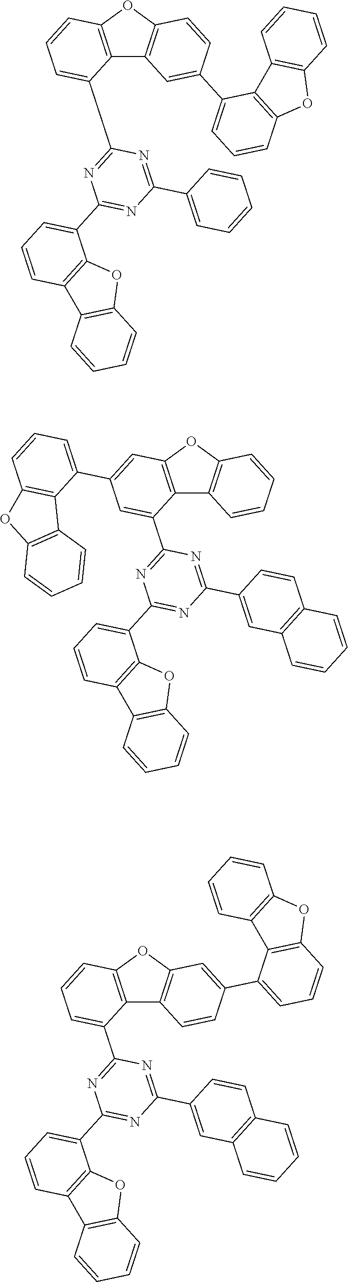 [SMILES: c1ccc(-c2nc(-c3cccc4c3oc3ccccc34)nc(-c3cccc4oc5ccc(-c6cccc7oc8ccccc8c67)cc5c34)n2)cc1 ; c1ccc2cc(-c3nc(-c4cccc5c4oc4ccccc45)nc(-c4cc(-c5cccc6oc7ccccc7c56)cc5oc6ccccc6c45)n3)ccc2c1 ; c1ccc2cc(-c3nc(-c4cccc5c4oc4ccccc45)nc(-c4cccc5oc6cc(-c7cccc8oc9ccccc9c78)ccc6c45)n3)ccc2c1]

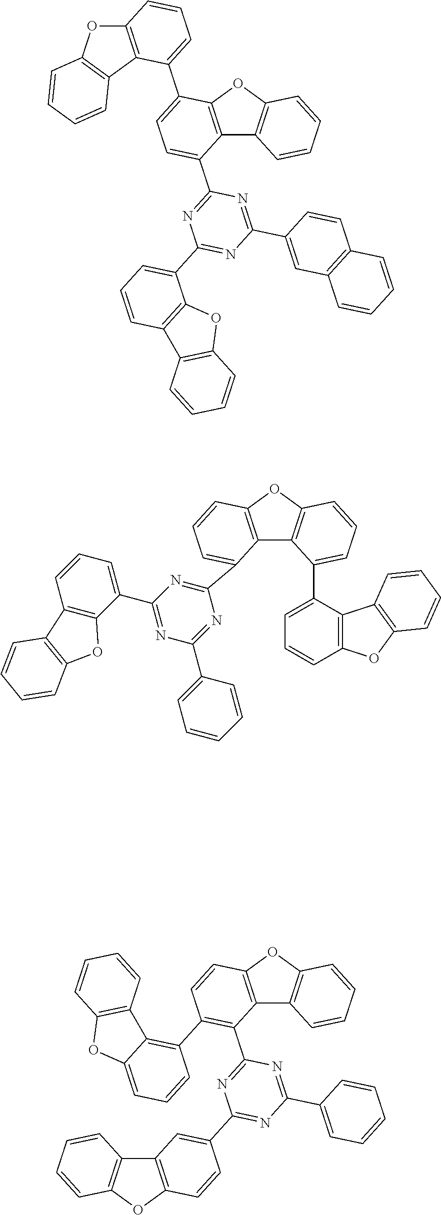 [SMILES: c1ccc2cc(-c3nc(-c4cccc5c4oc4ccccc45)nc(-c4ccc(-c5cccc6oc7ccccc7c56)c5oc6ccccc6c45)n3)ccc2c1 ; c1ccc(-c2nc(-c3cccc4c3oc3ccccc34)nc(-c3cccc4oc5cccc(-c6cccc7oc8ccccc8c67)c5c34)n2)cc1 ; c1ccc(-c2nc(-c3ccc4oc5ccccc5c4c3)nc(-c3c(-c4cccc5oc6ccccc6c45)ccc4oc5ccccc5c34)n2)cc1]

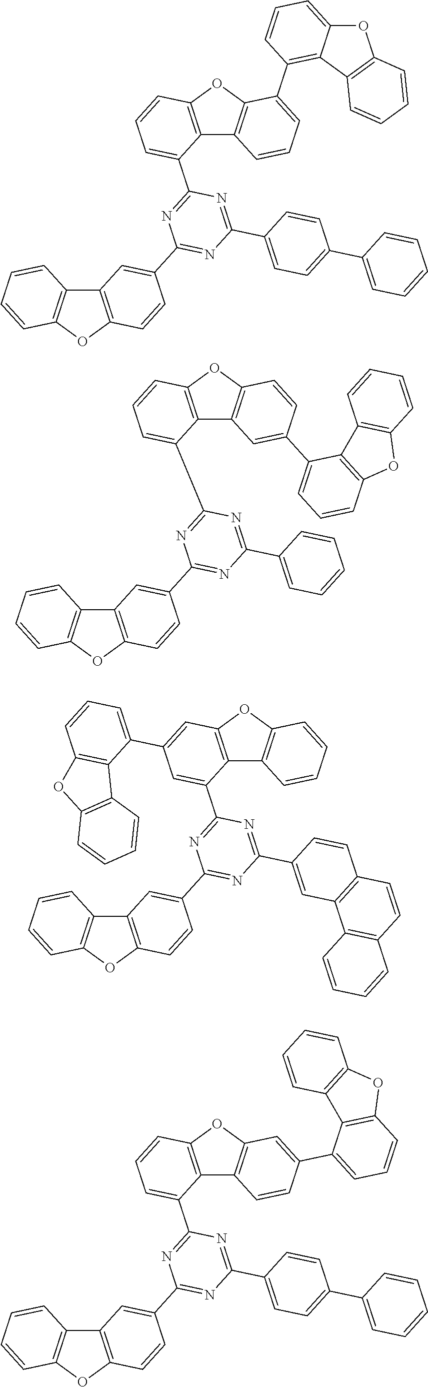

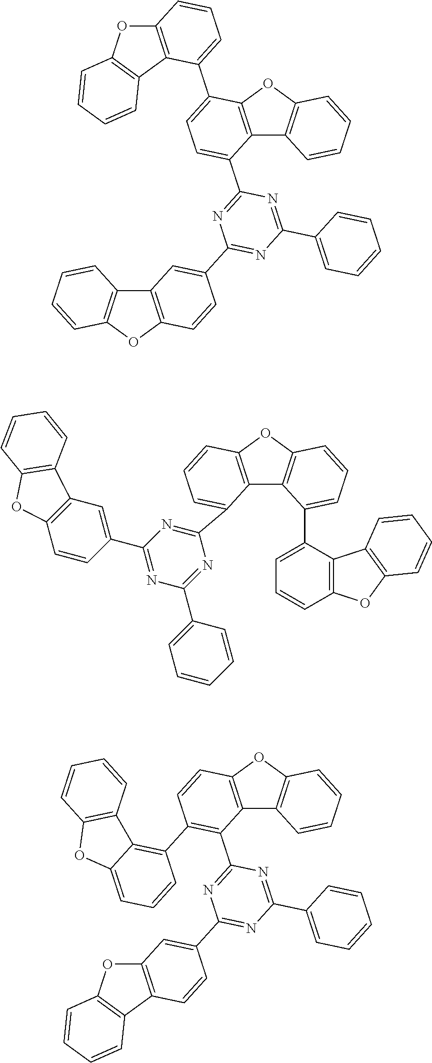 [SMILES: c1ccc(-c2nc(-c3ccc4oc5ccccc5c4c3)nc(-c3ccc(-c4cccc5oc6ccccc6c45)c4oc5ccccc5c34)n2)cc1 ; c1ccc(-c2nc(-c3ccc4oc5ccccc5c4c3)nc(-c3cccc4oc5cccc(-c6cccc7oc8ccccc8c67)c5c34)n2)cc1 ; c1ccc(-c2nc(-c3ccc4c(c3)oc3ccccc34)nc(-c3c(-c4cccc5oc6ccccc6c45)ccc4oc5ccccc5c34)n2)cc1]

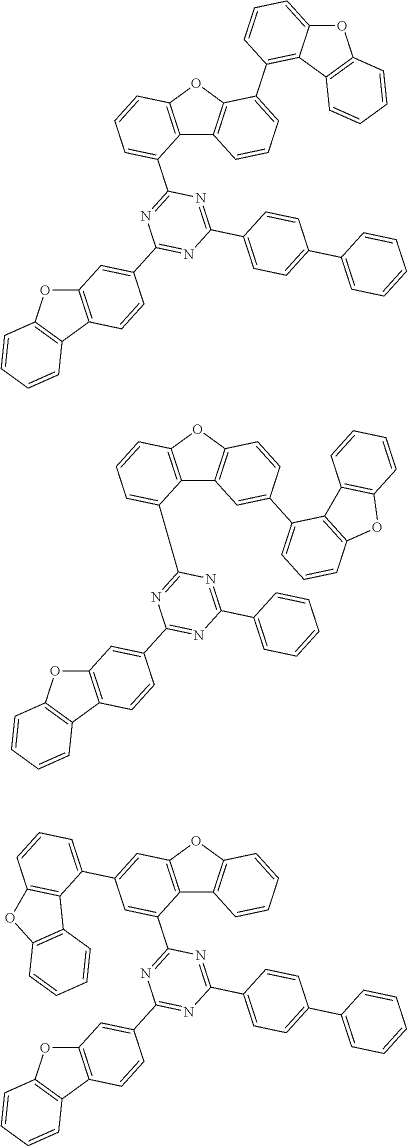 [SMILES: c1ccc(-c2ccc(-c3nc(-c4ccc5c(c4)oc4ccccc45)nc(-c4cccc5oc6c(-c7cccc8oc9ccccc9c78)cccc6c45)n3)cc2)cc1 ; c1ccc(-c2nc(-c3ccc4c(c3)oc3ccccc34)nc(-c3cccc4oc5ccc(-c6cccc7oc8ccccc8c67)cc5c34)n2)cc1 ; c1ccc(-c2ccc(-c3nc(-c4ccc5c(c4)oc4ccccc45)nc(-c4cc(-c5cccc6oc7ccccc7c56)cc5oc6ccccc6c45)n3)cc2)cc1]

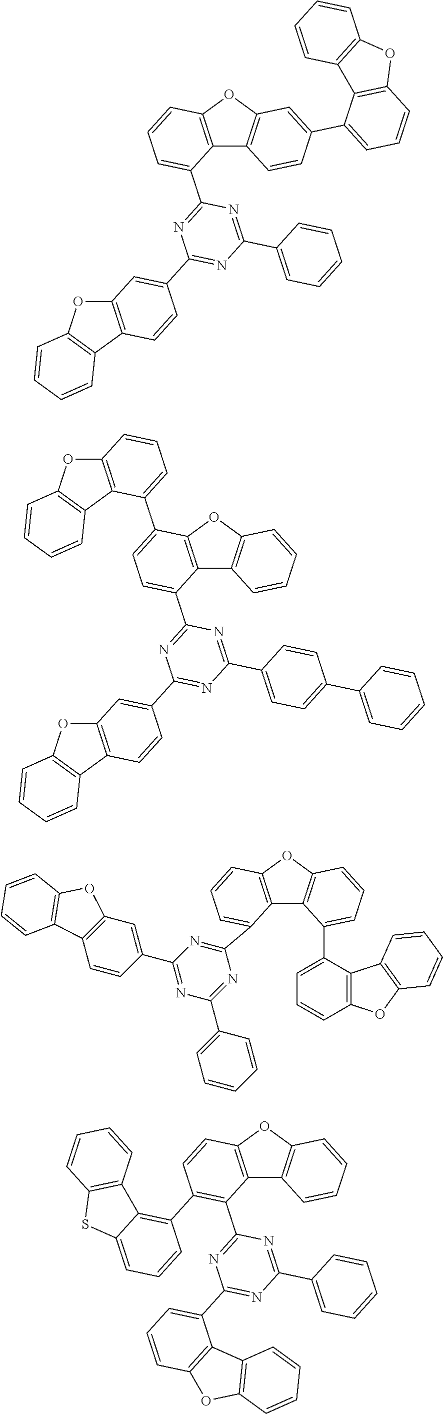

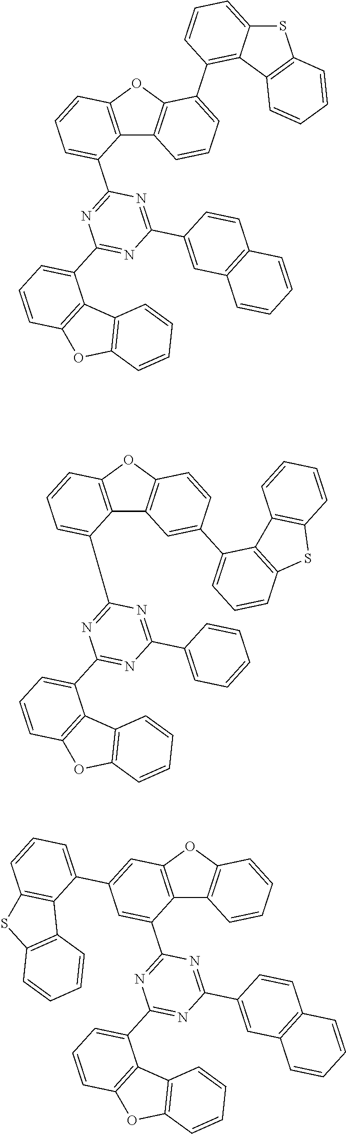 [SMILES: c1ccc2cc(-c3nc(-c4cccc5oc6ccccc6c45)nc(-c4cccc5oc6c(-c7cccc8sc9ccccc9c78)cccc6c45)n3)ccc2c1 ; c1ccc(-c2nc(-c3cccc4oc5ccccc5c34)nc(-c3cccc4oc5ccc(-c6cccc7sc8ccccc8c67)cc5c34)n2)cc1 ; c1ccc2cc(-c3nc(-c4cccc5oc6ccccc6c45)nc(-c4cc(-c5cccc6sc7ccccc7c56)cc5oc6ccccc6c45)n3)ccc2c1]

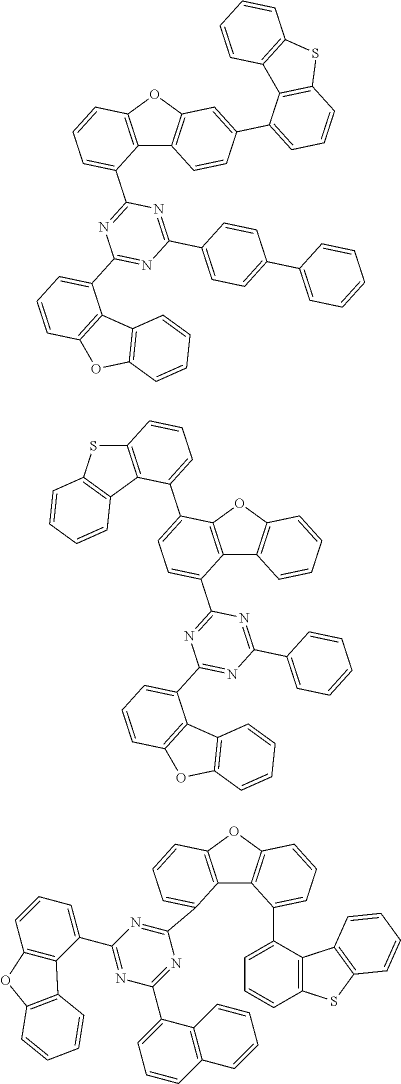

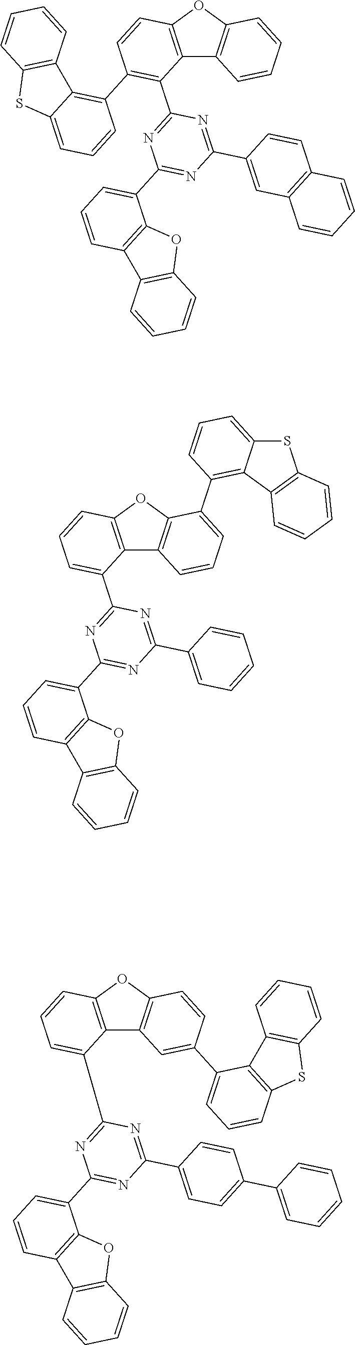 [SMILES: c1ccc2cc(-c3nc(-c4cccc5c4oc4ccccc45)nc(-c4c(-c5cccc6sc7ccccc7c56)ccc5oc6ccccc6c45)n3)ccc2c1 ; c1ccc(-c2nc(-c3cccc4c3oc3ccccc34)nc(-c3cccc4oc5c(-c6cccc7sc8ccccc8c67)cccc5c34)n2)cc1 ; c1ccc(-c2ccc(-c3nc(-c4cccc5c4oc4ccccc45)nc(-c4cccc5oc6ccc(-c7cccc8sc9ccccc9c78)cc6c45)n3)cc2)cc1]

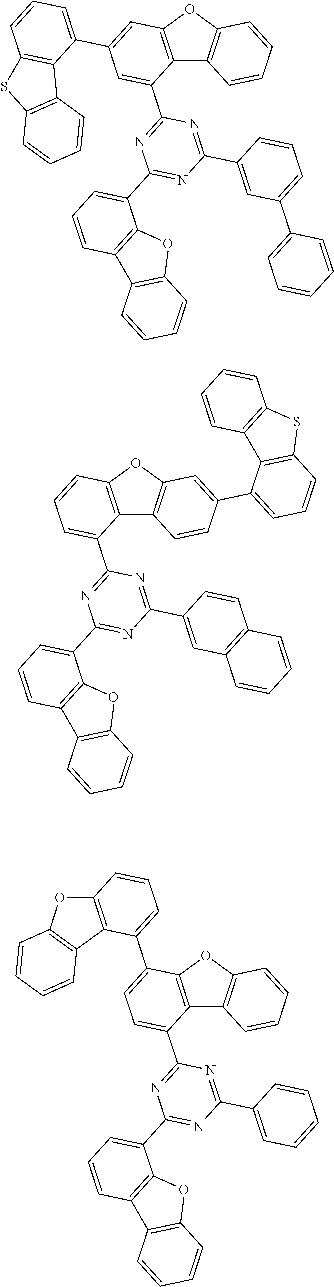 [SMILES: c1ccc(-c2cccc(-c3nc(-c4cccc5c4oc4ccccc45)nc(-c4cc(-c5cccc6sc7ccccc7c56)cc5oc6ccccc6c45)n3)c2)cc1 ; c1ccc2cc(-c3nc(-c4cccc5c4oc4ccccc45)nc(-c4cccc5oc6cc(-c7cccc8sc9ccccc9c78)ccc6c45)n3)ccc2c1 ; c1ccc(-c2nc(-c3cccc4c3oc3ccccc34)nc(-c3ccc(-c4cccc5oc6ccccc6c45)c4oc5ccccc5c34)n2)cc1]

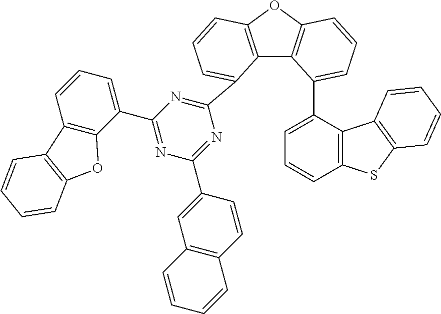 [SMILES: c1ccc2cc(-c3nc(-c4cccc5c4oc4ccccc45)nc(-c4cccc5oc6cccc(-c7cccc8sc9ccccc9c78)c6c45)n3)ccc2c1]

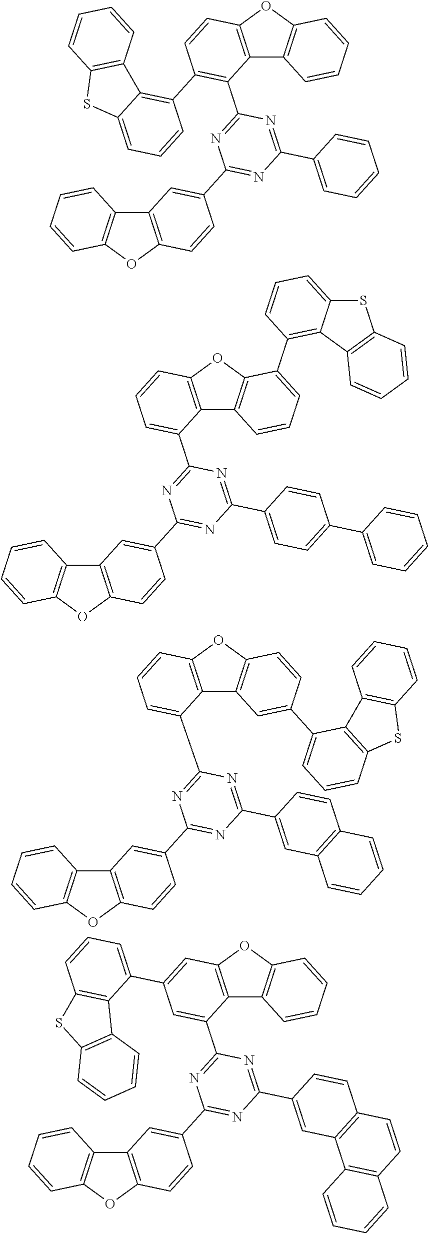

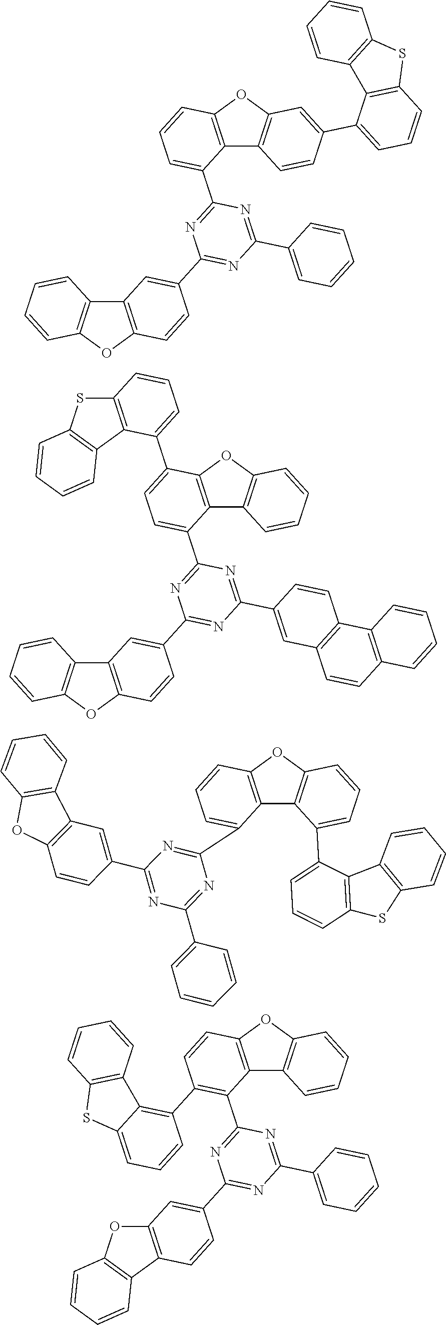

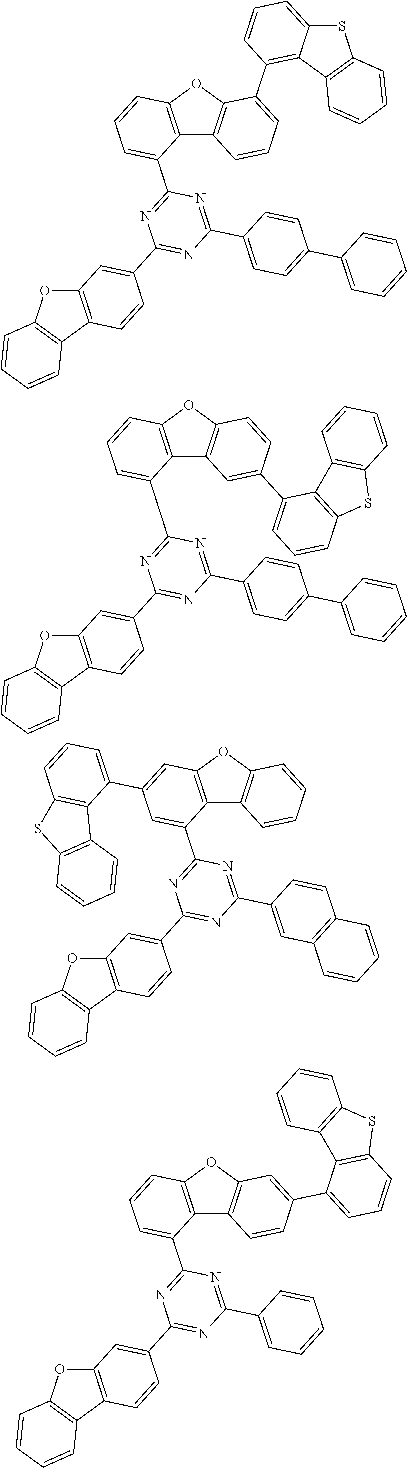

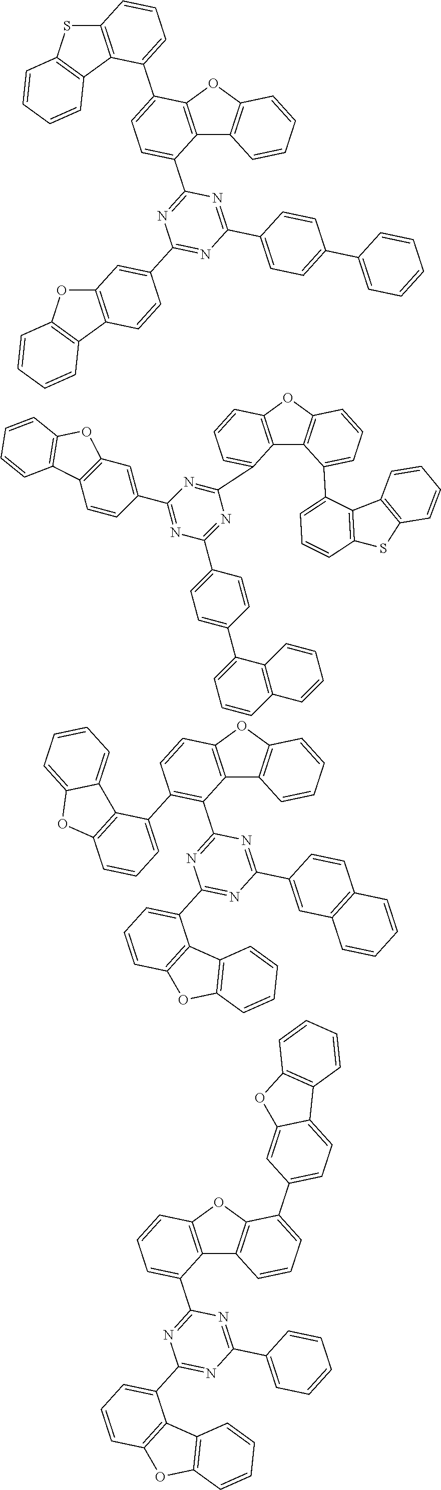

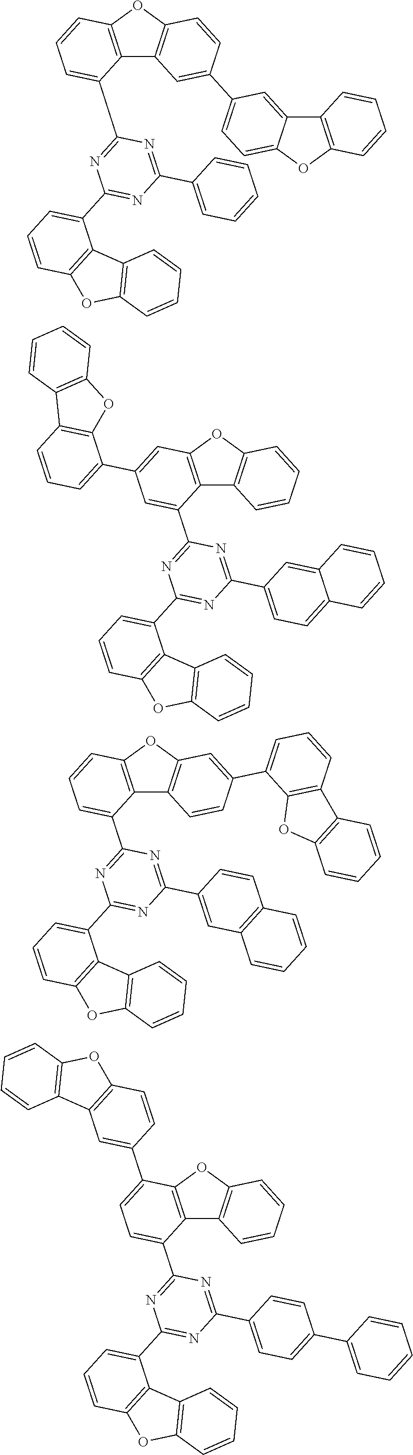

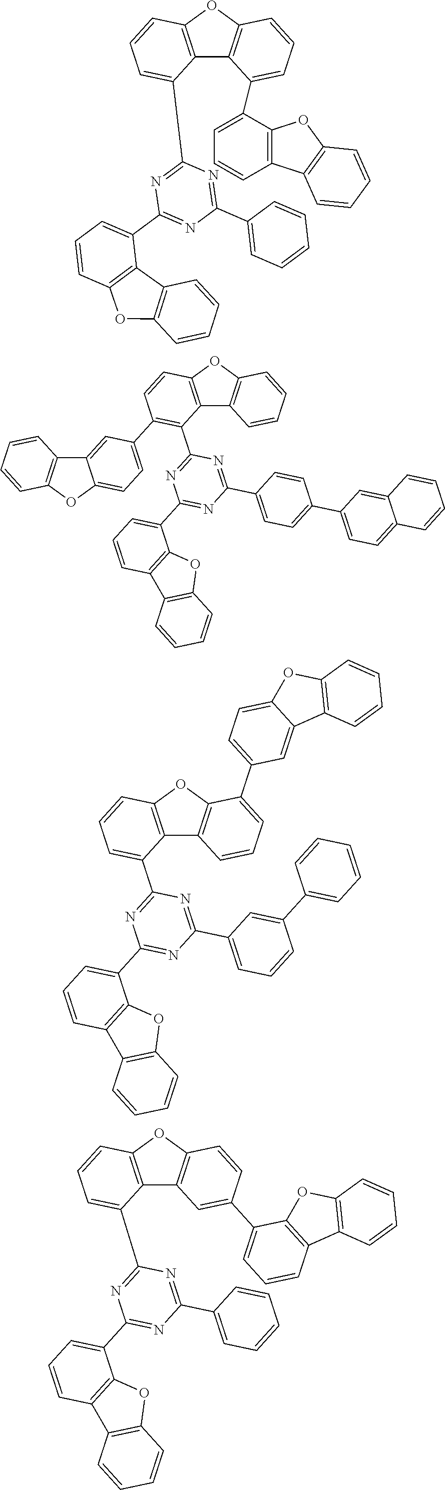

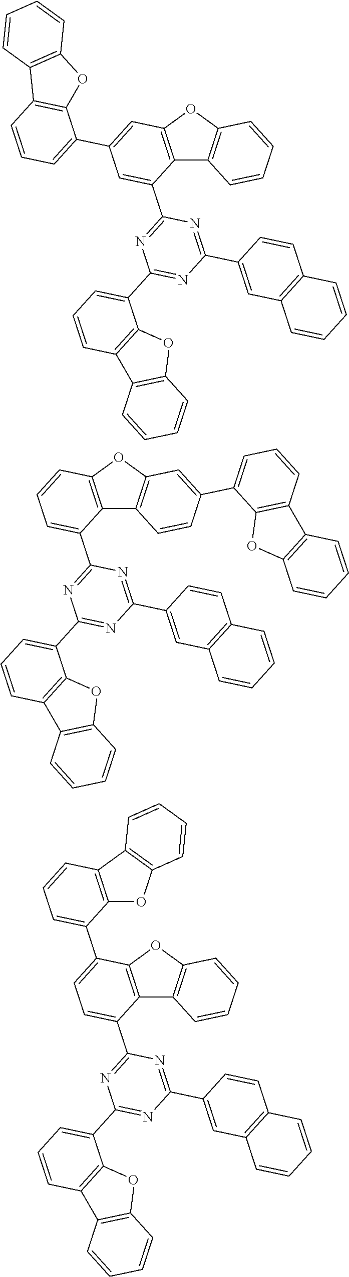

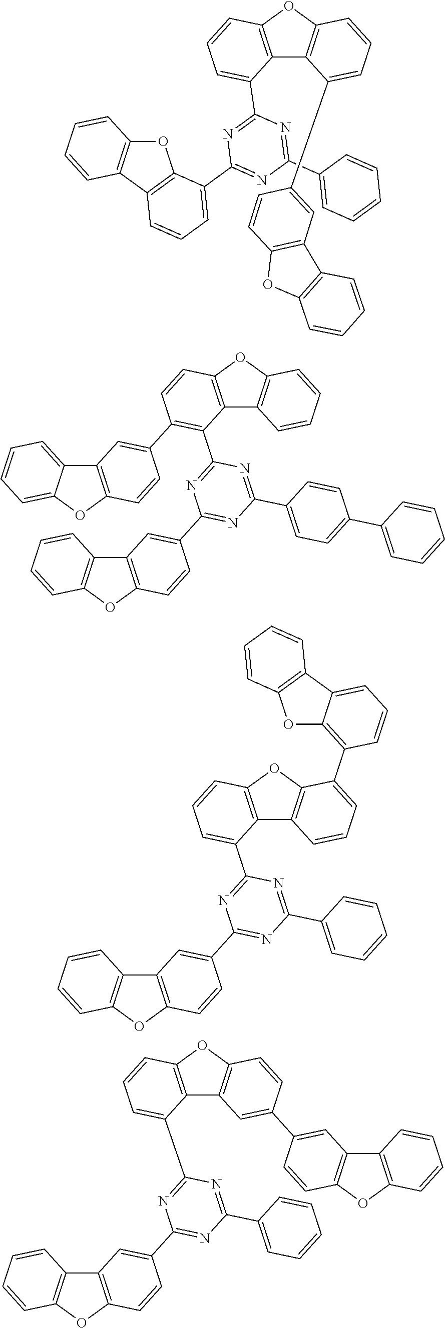

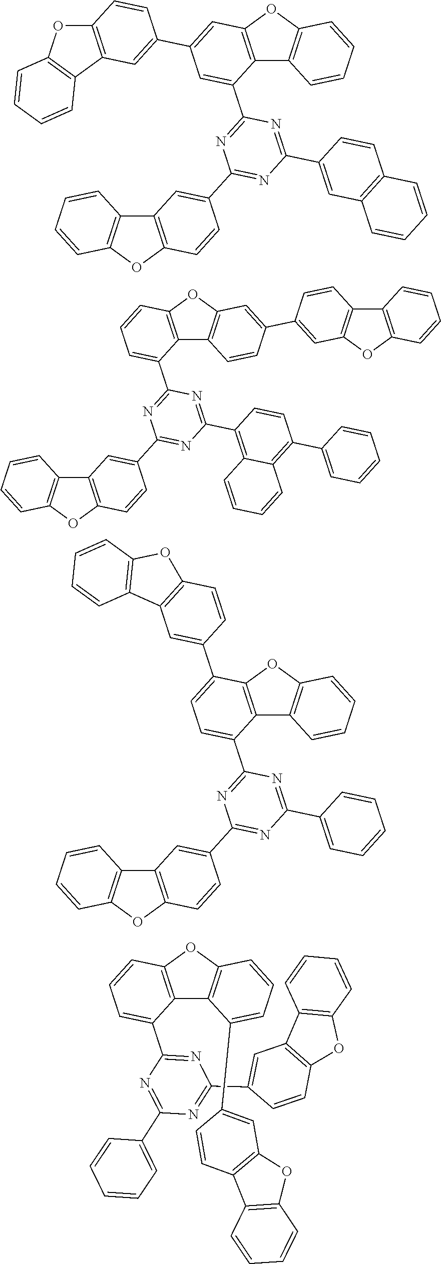

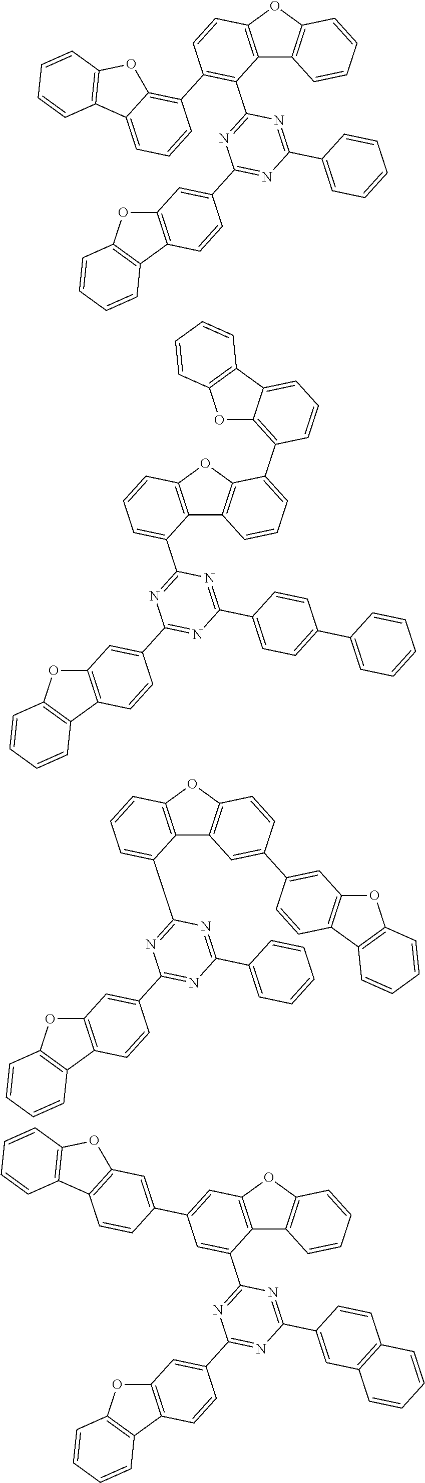

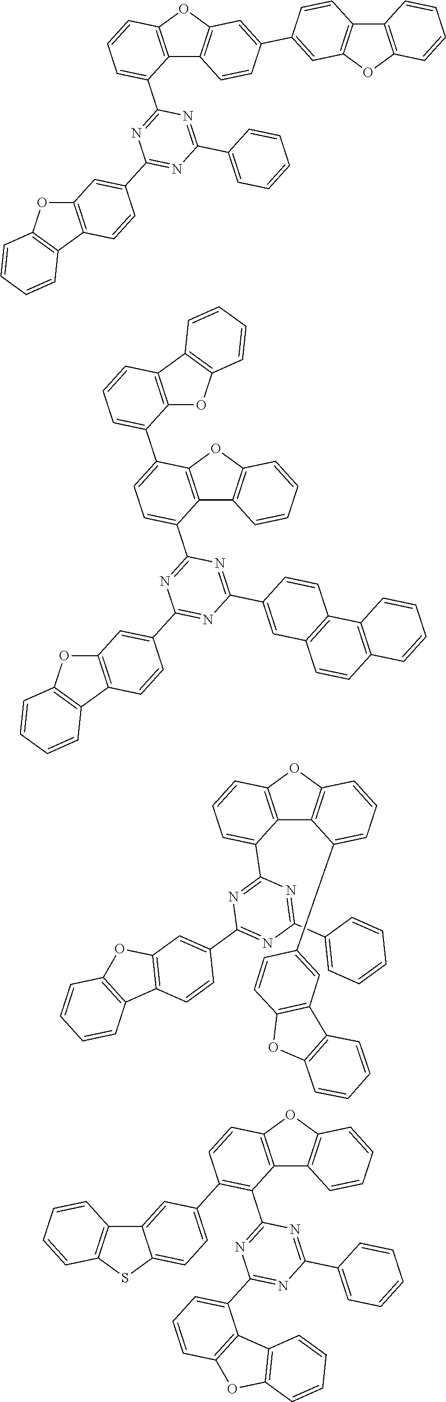

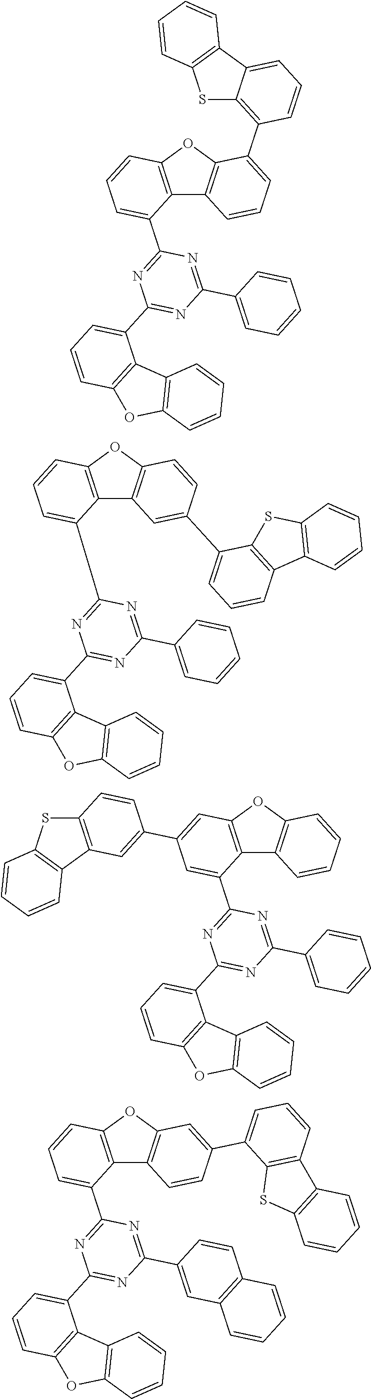

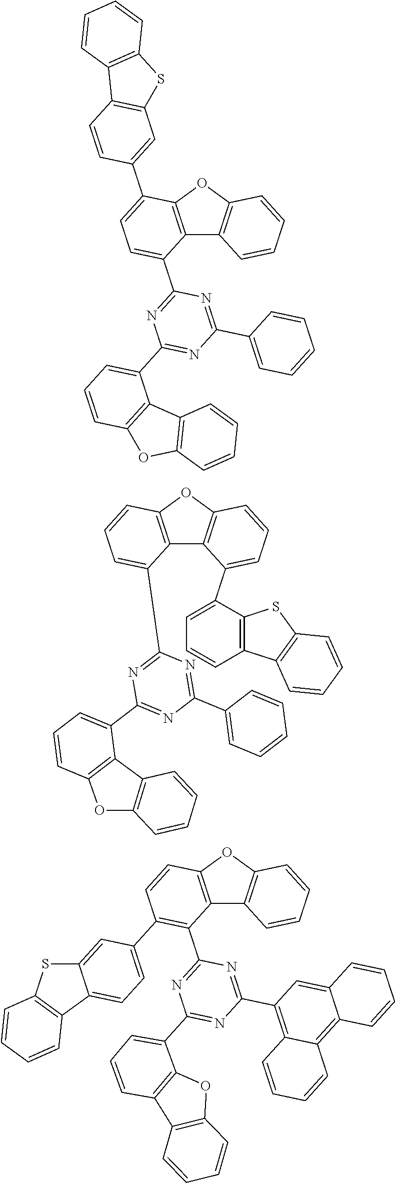

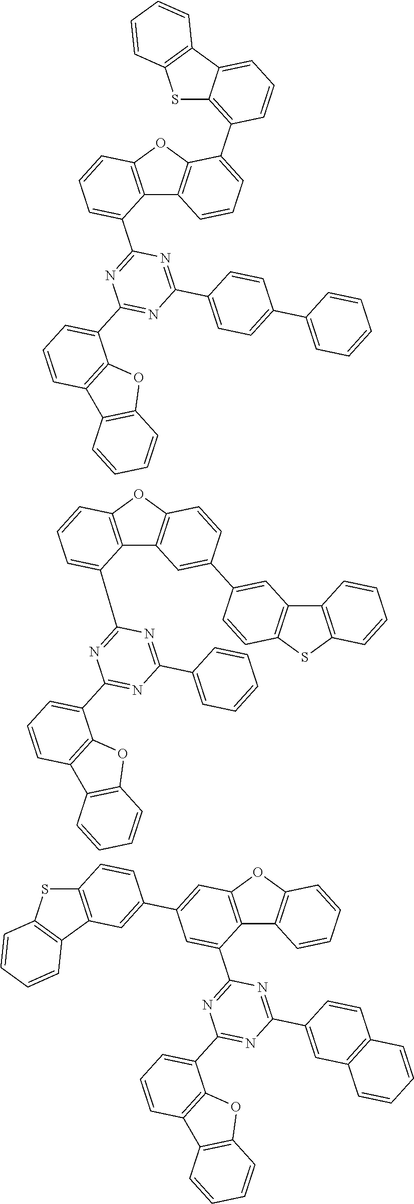

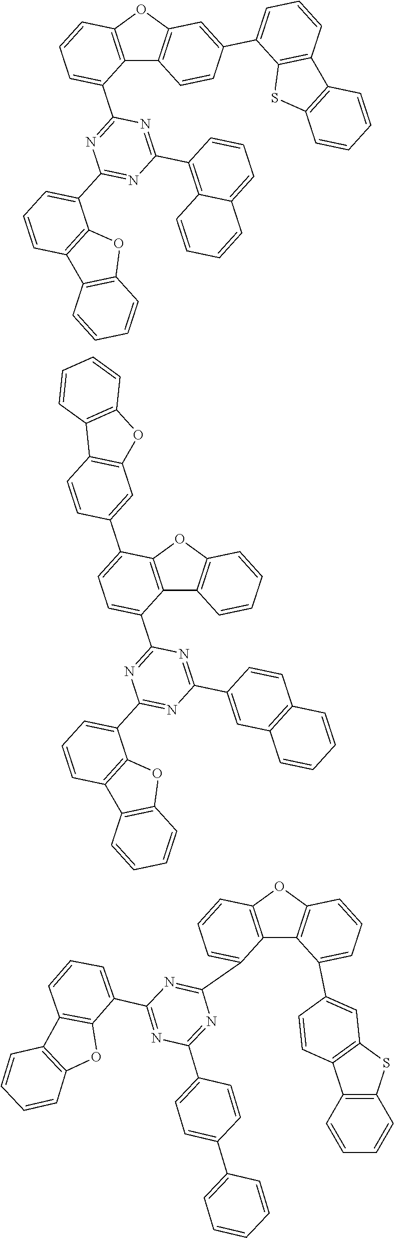

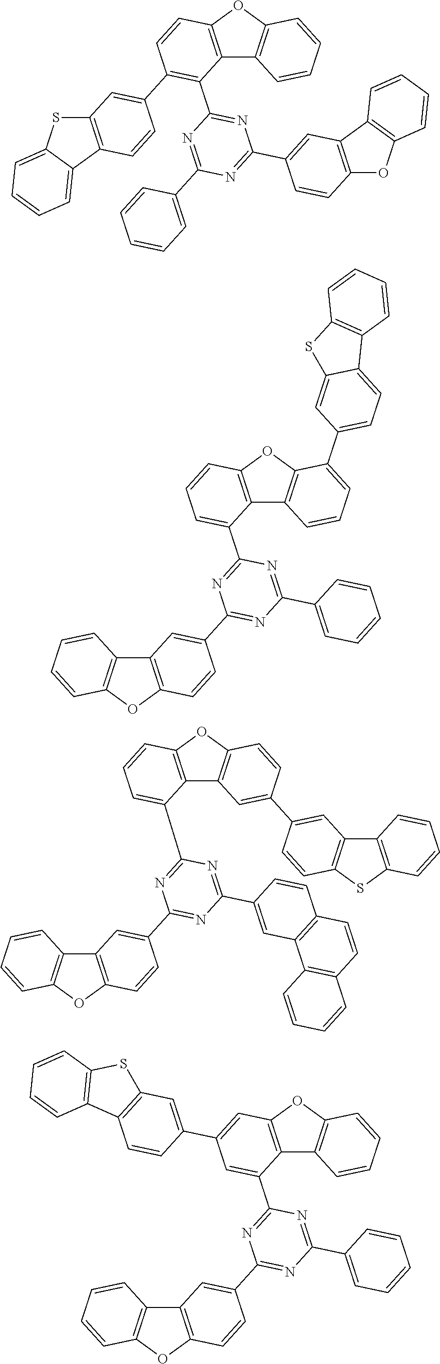

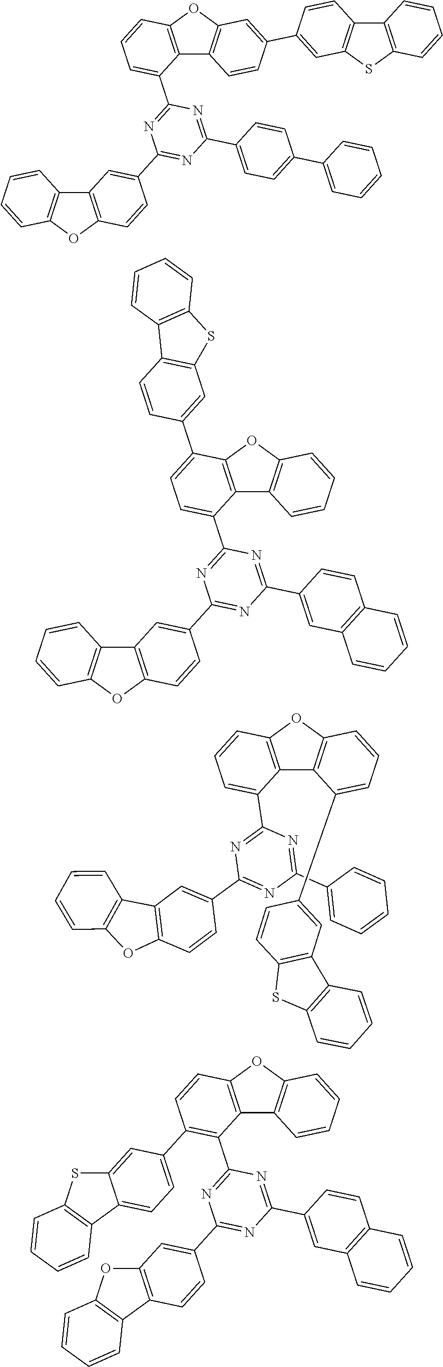

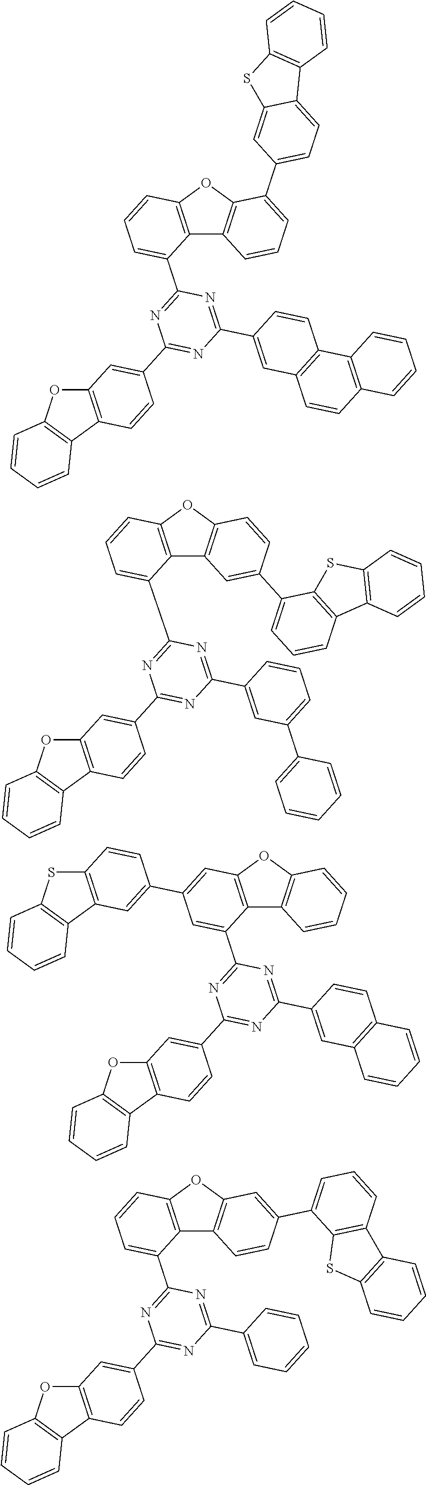

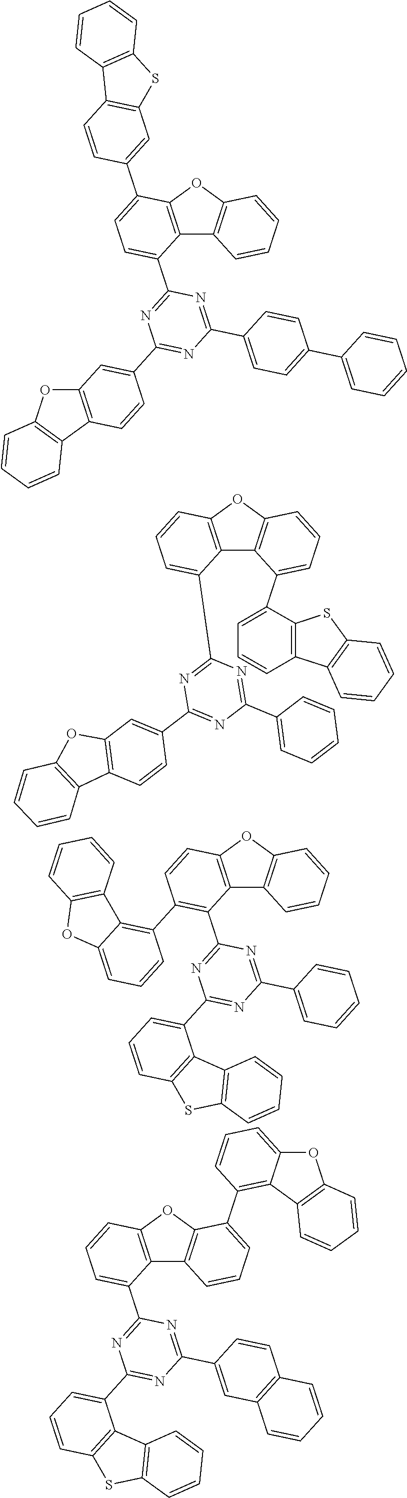

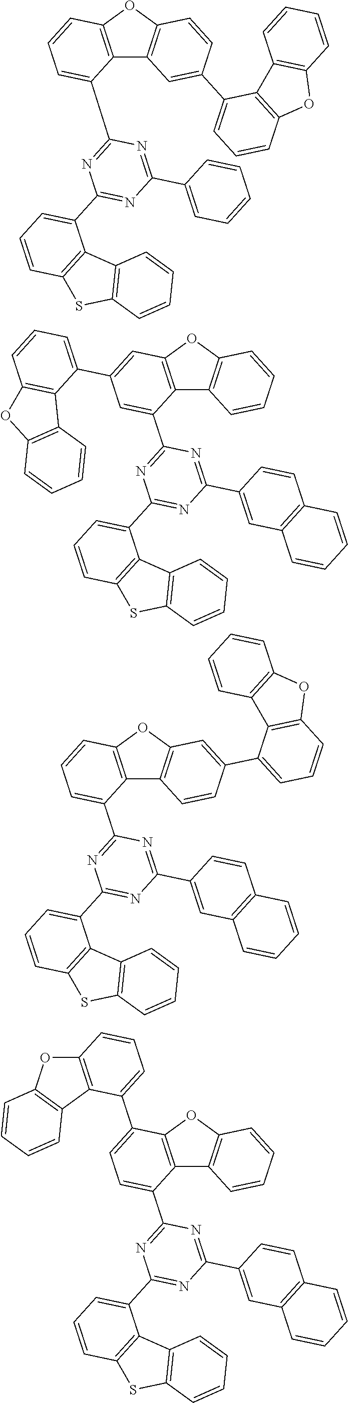

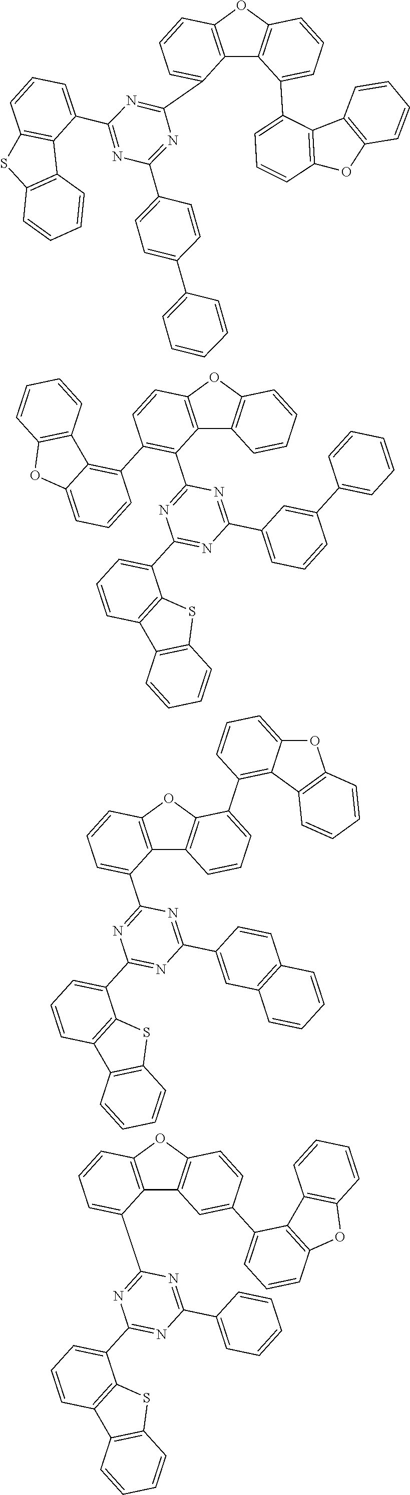

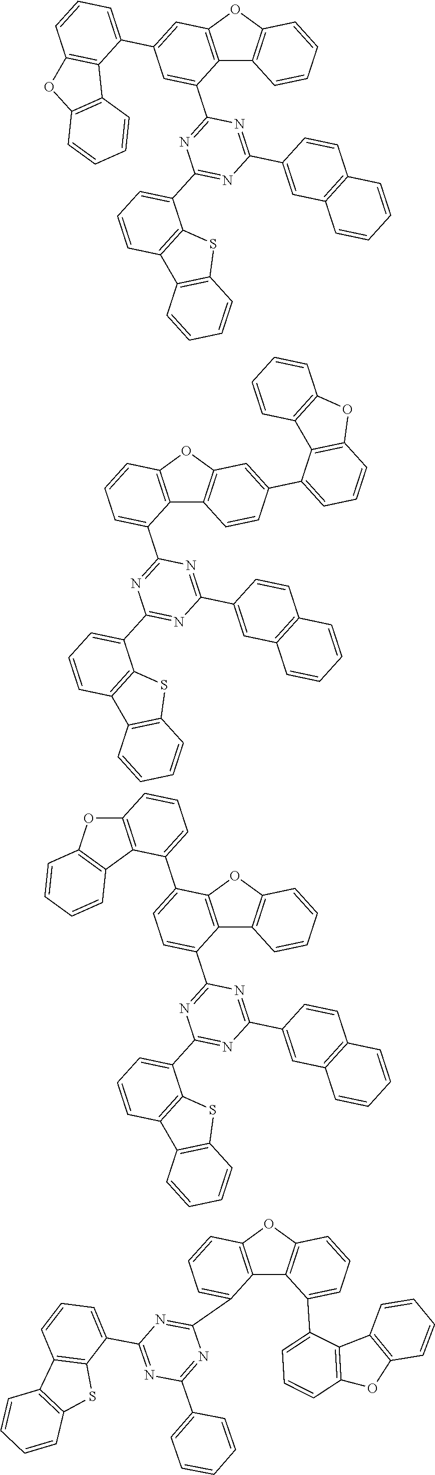

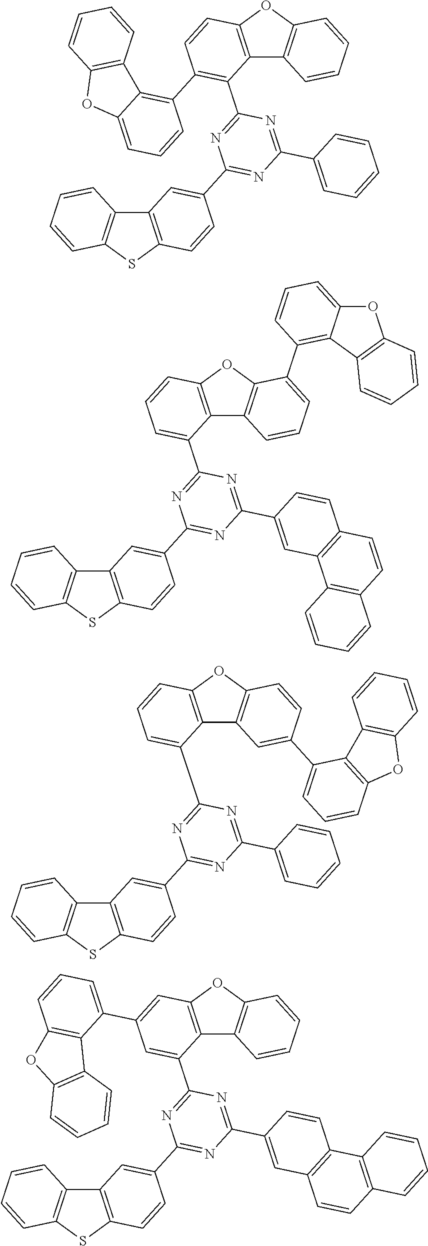

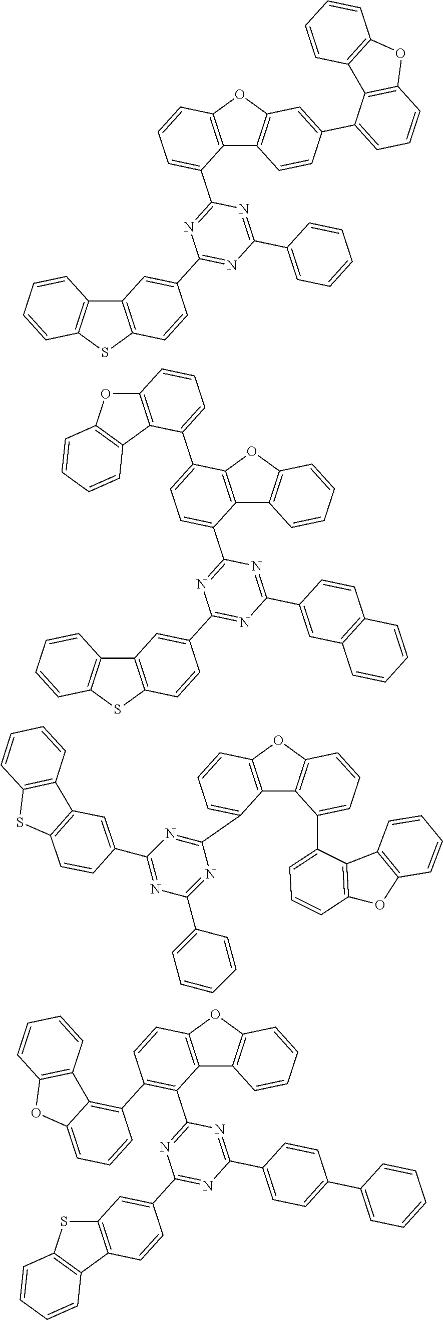

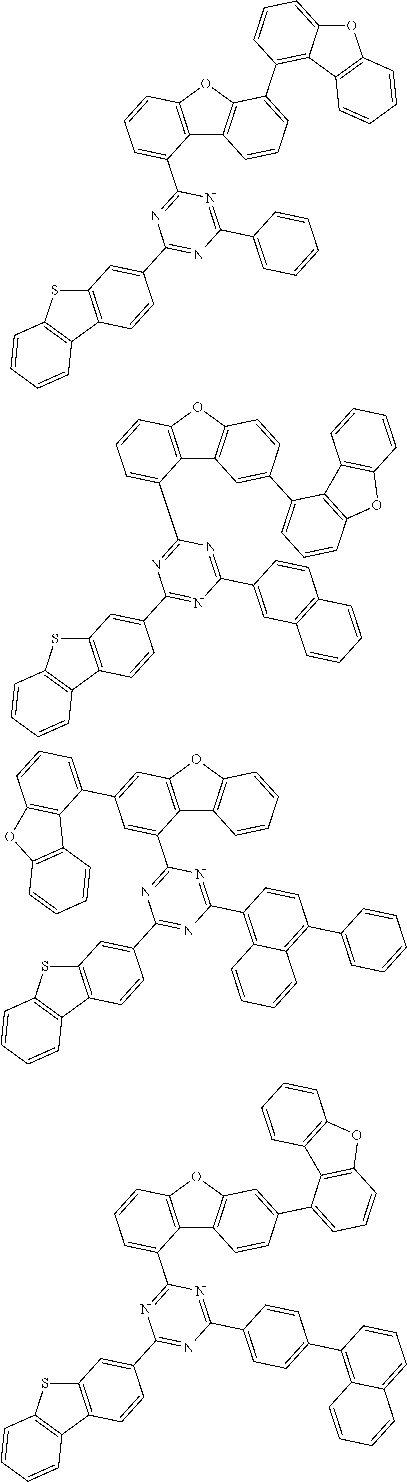

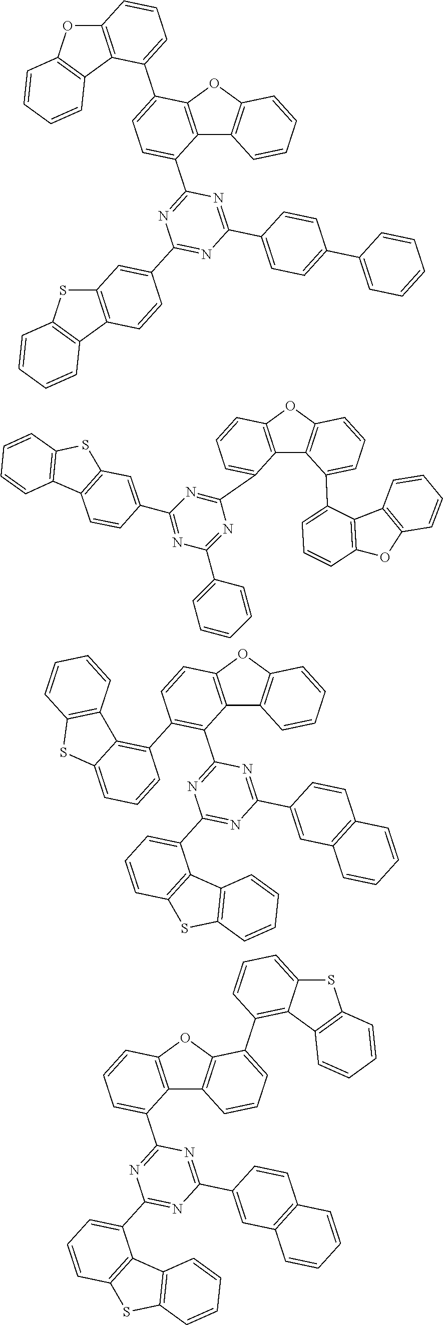

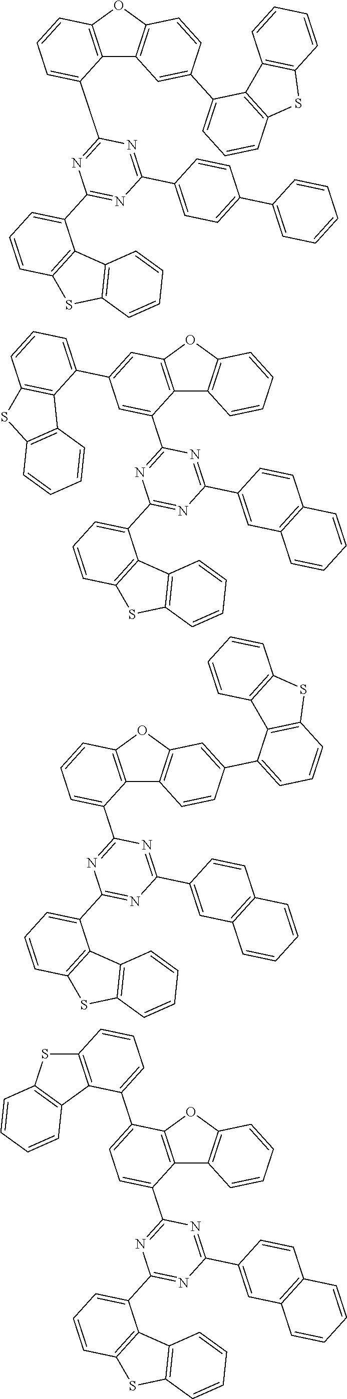

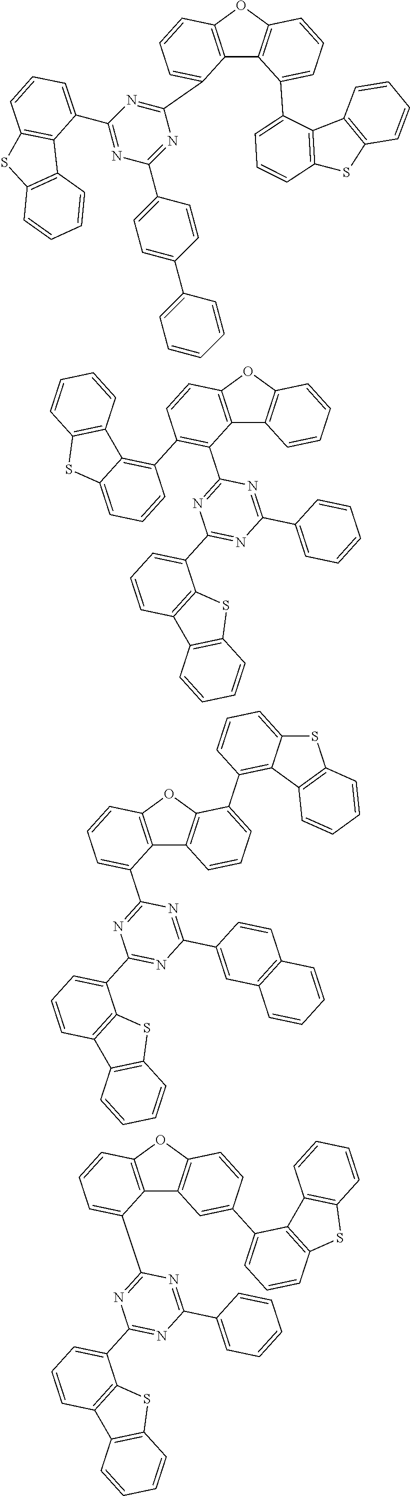

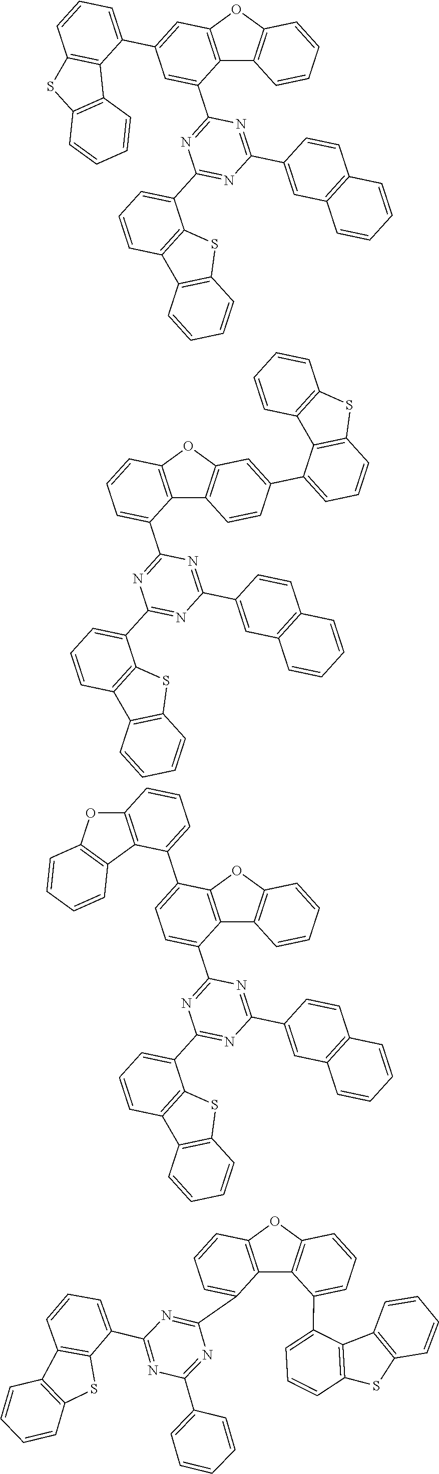

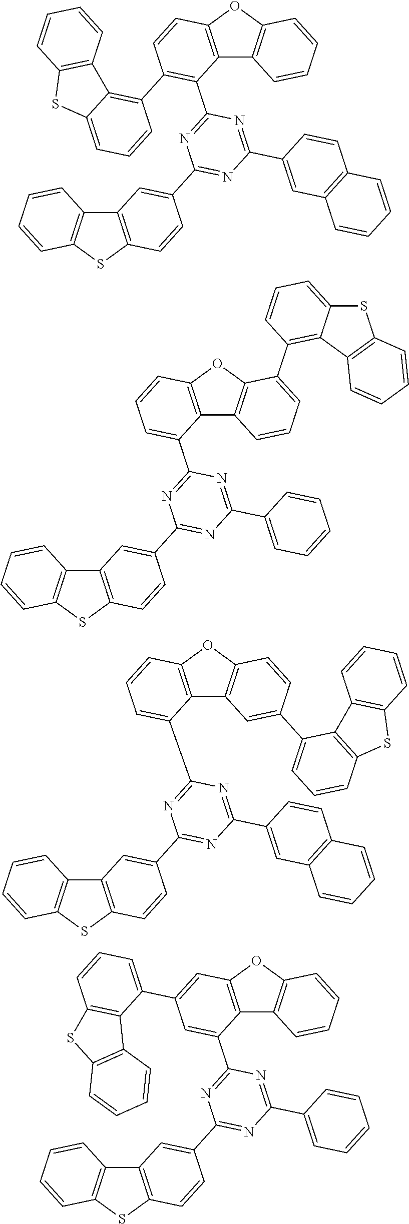

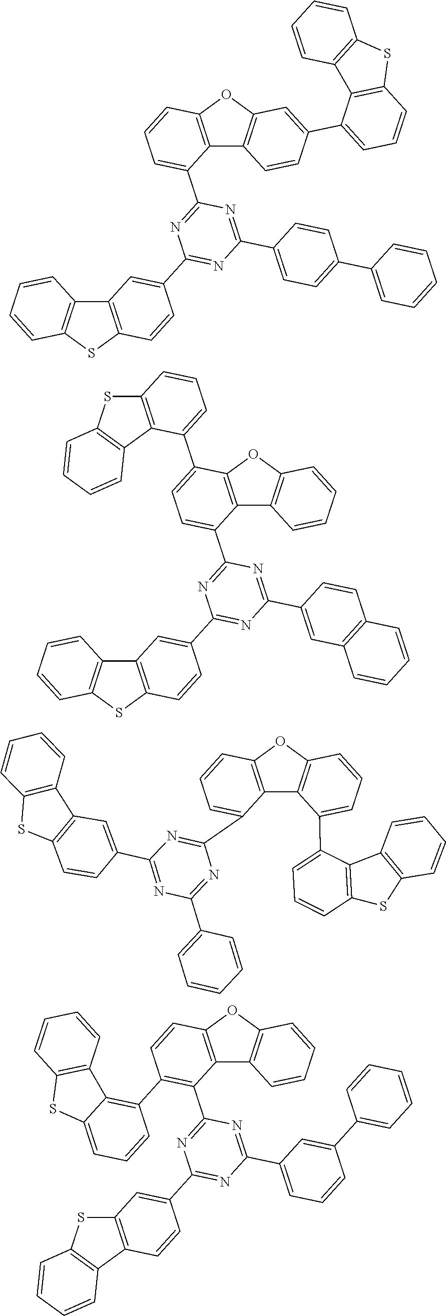

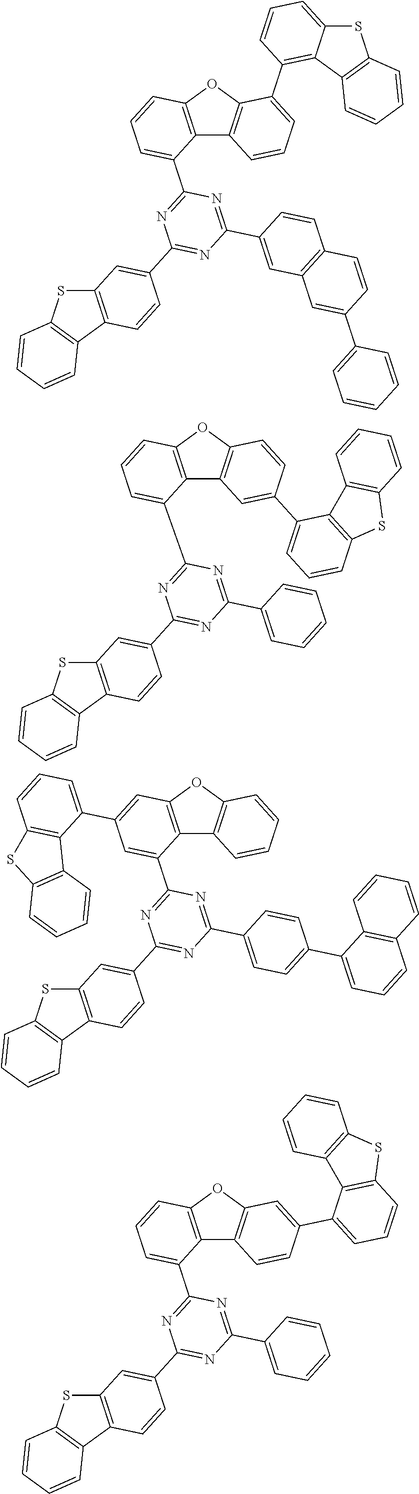

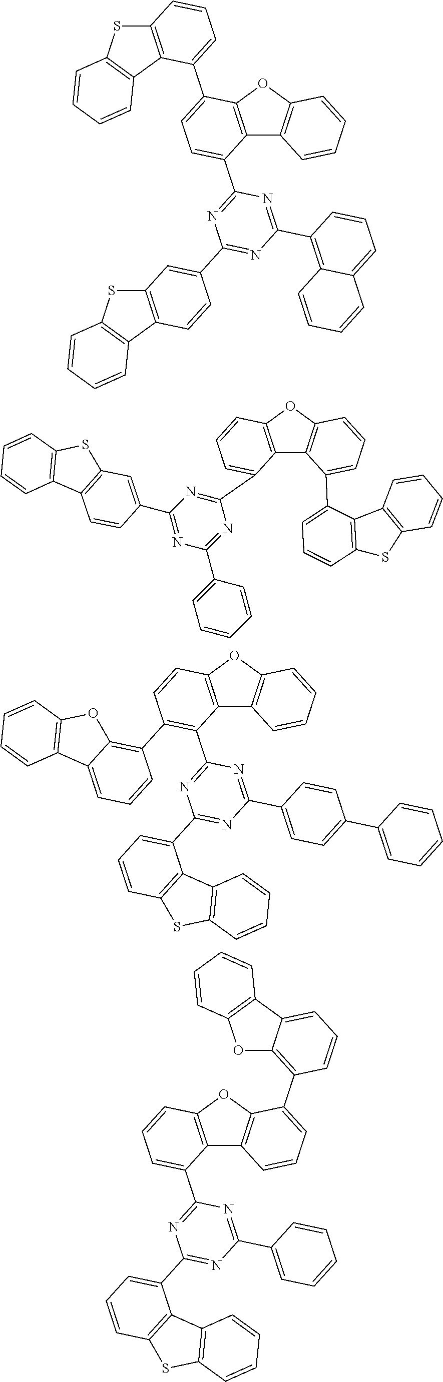

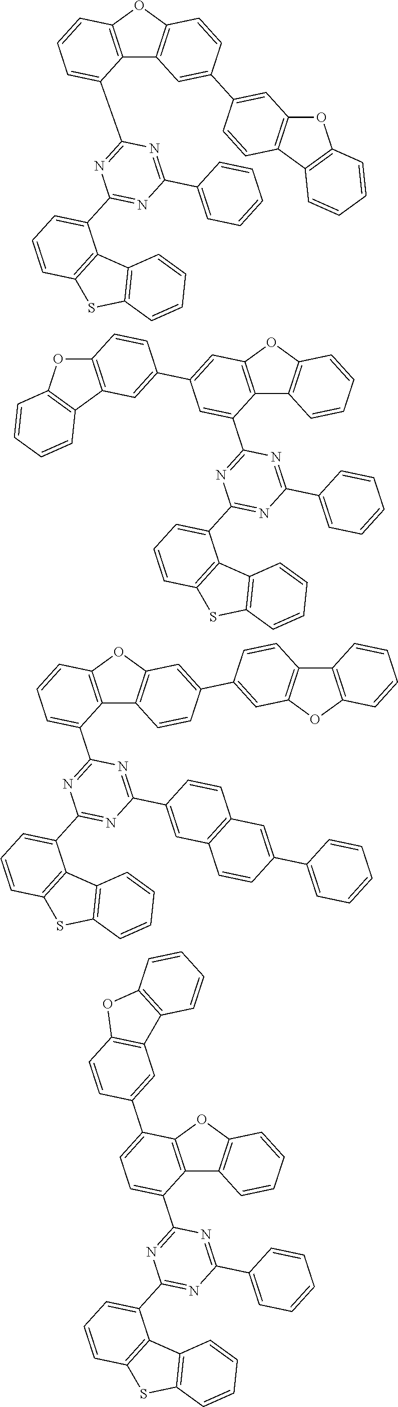

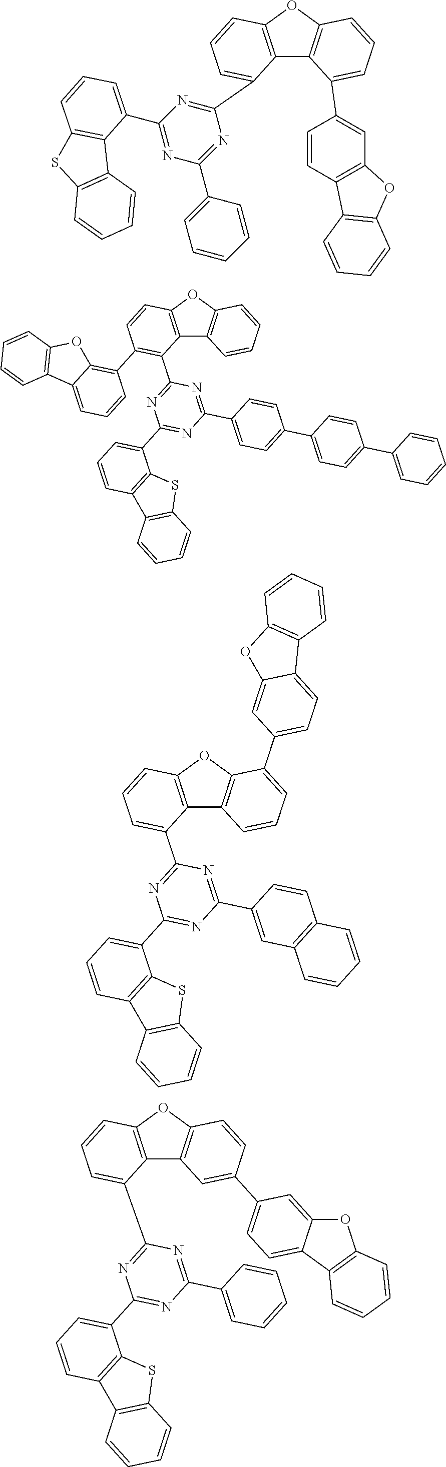

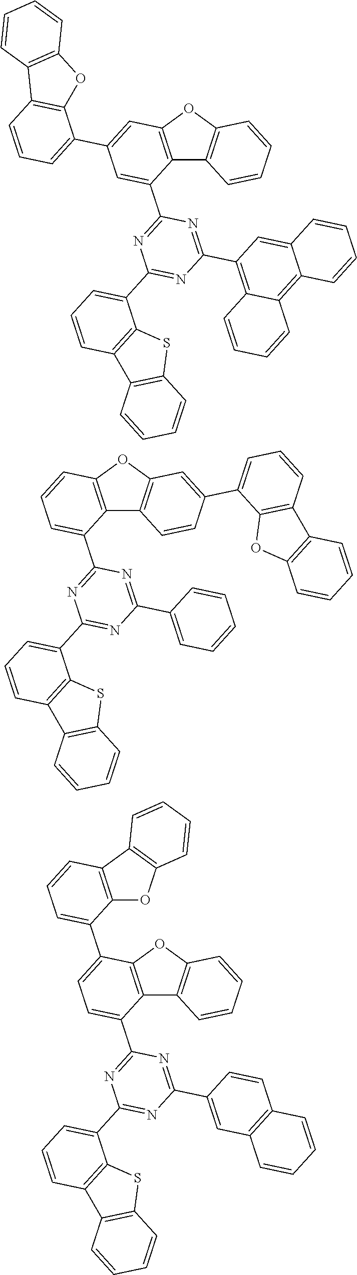

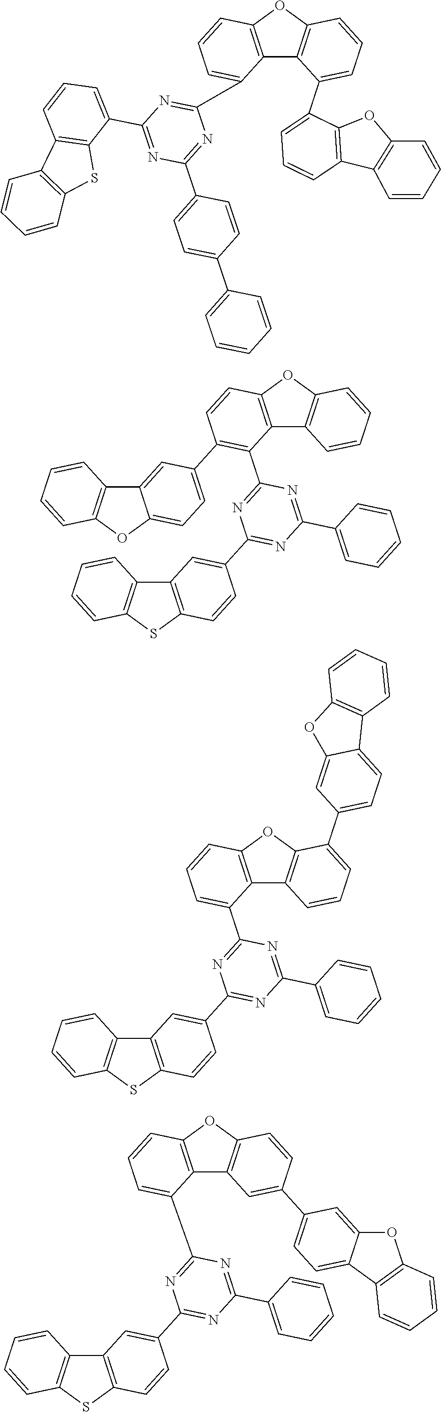

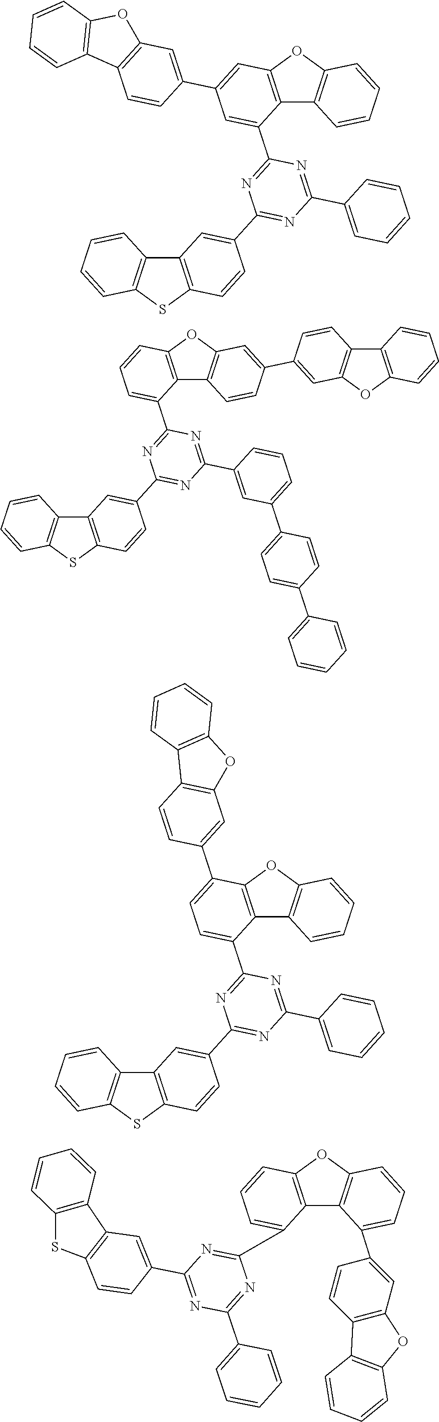

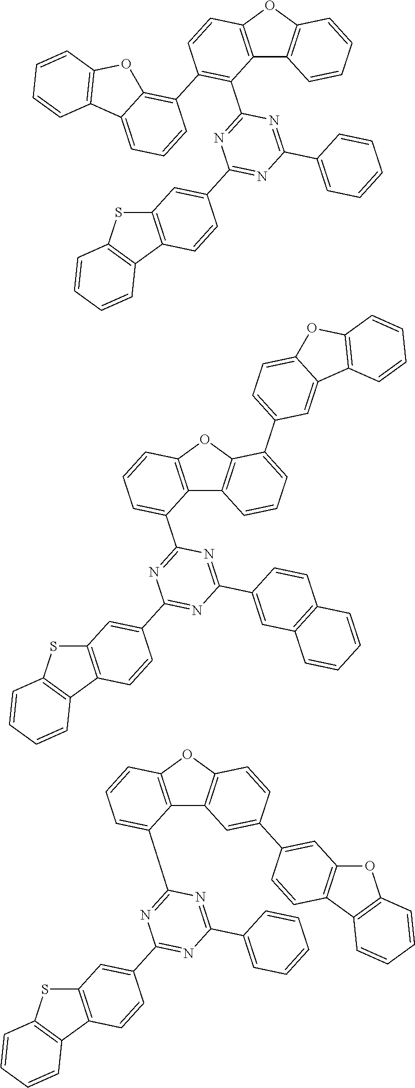 [SMILES: c1ccc(-c2nc(-c3ccc4c(c3)sc3ccccc34)nc(-c3c(-c4cccc5c4oc4ccccc45)ccc4oc5ccccc5c34)n2)cc1 ; c1ccc2cc(-c3nc(-c4ccc5c(c4)sc4ccccc45)nc(-c4cccc5oc6c(-c7ccc8oc9ccccc9c8c7)cccc6c45)n3)ccc2c1 ; c1ccc(-c2nc(-c3ccc4c(c3)sc3ccccc34)nc(-c3cccc4oc5ccc(-c6ccc7c(c6)oc6ccccc67)cc5c34)n2)cc1]

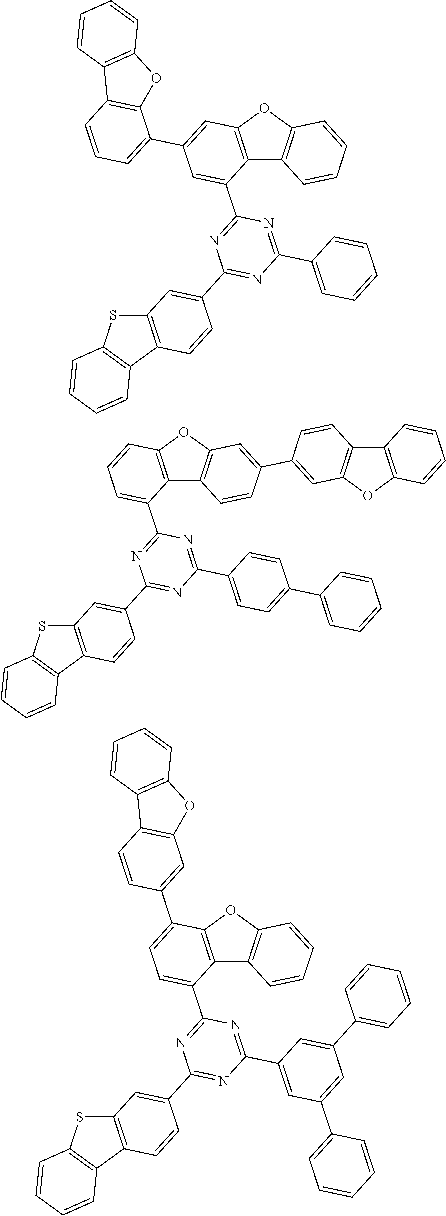

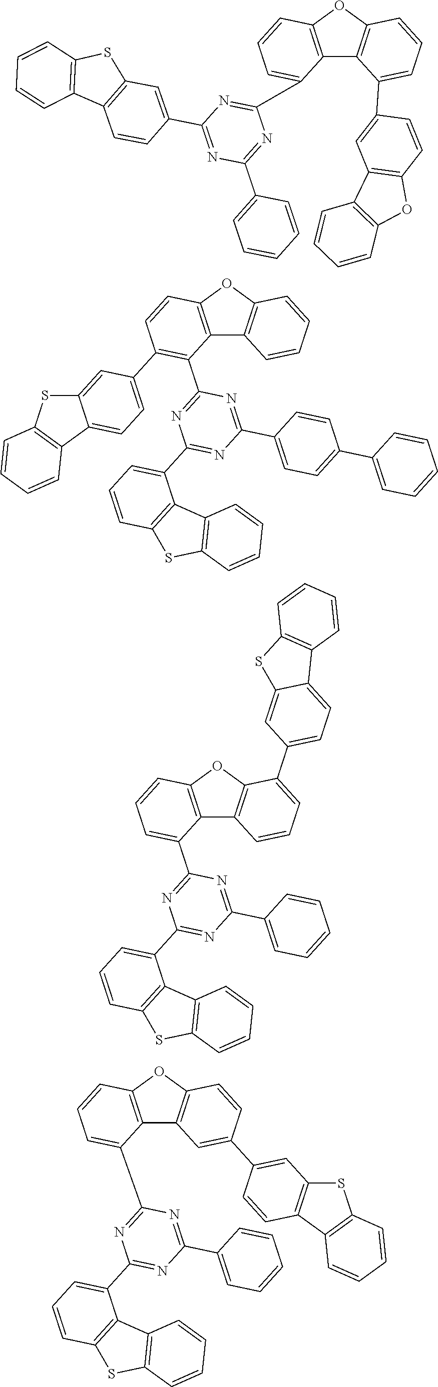

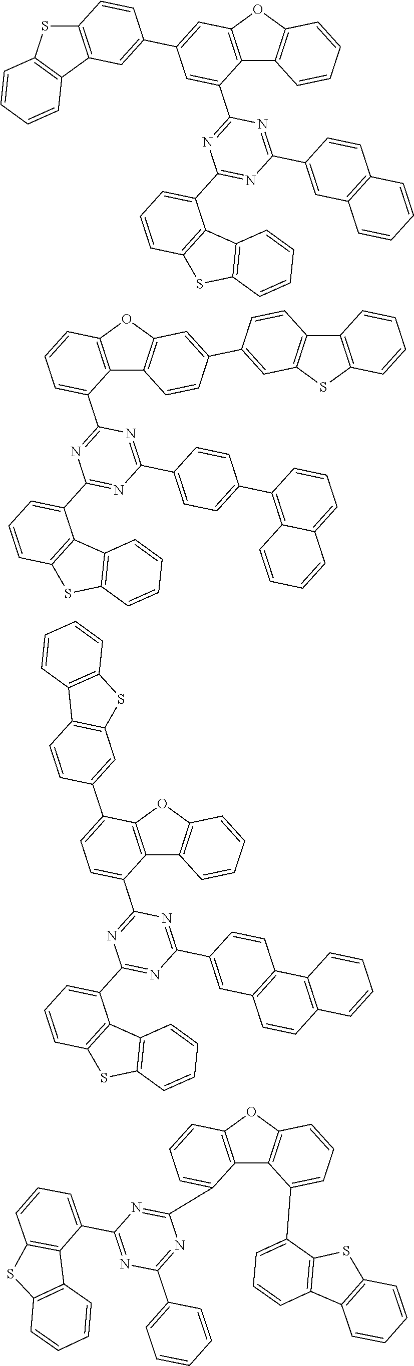

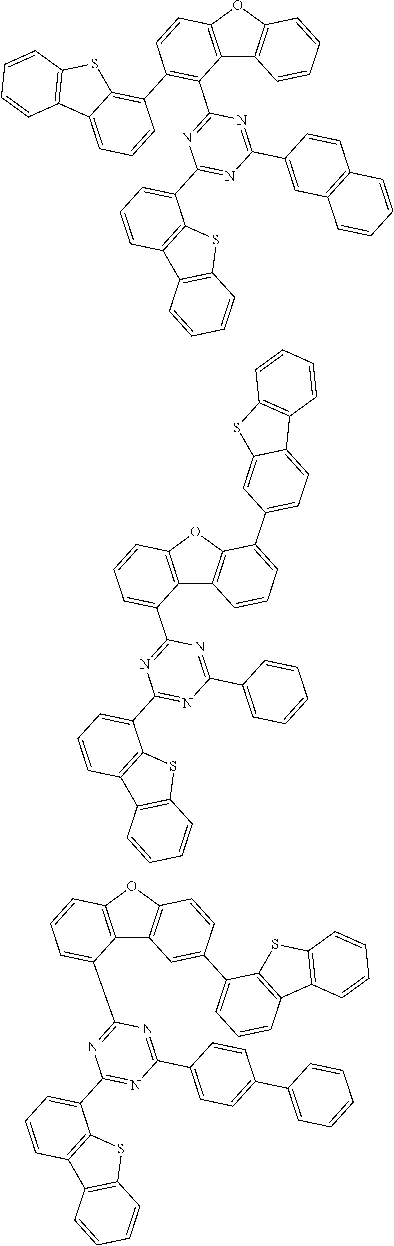

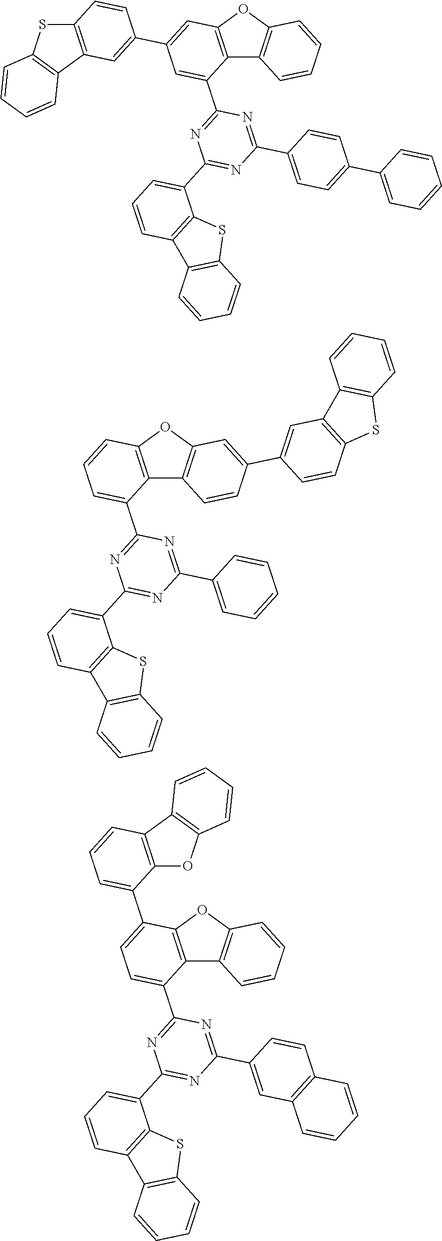

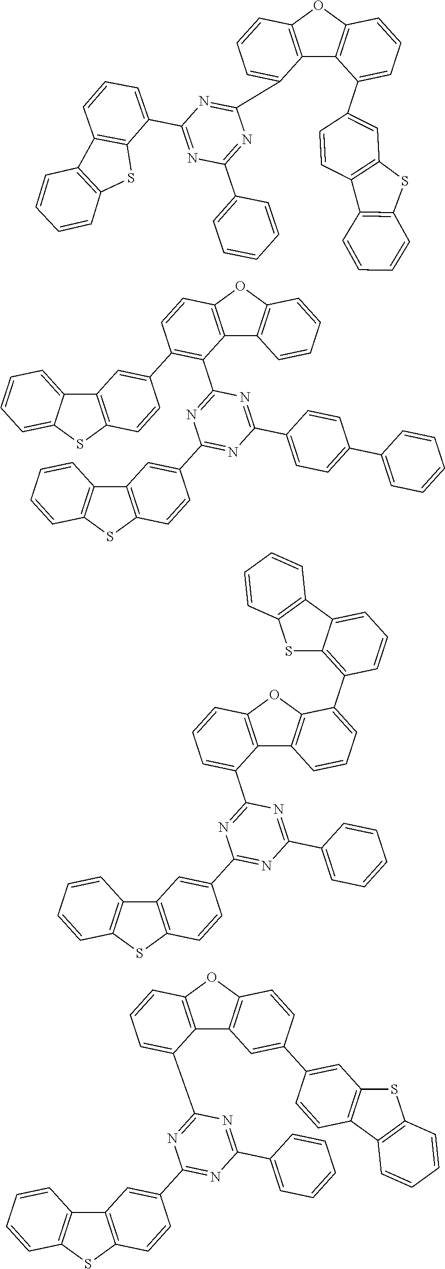

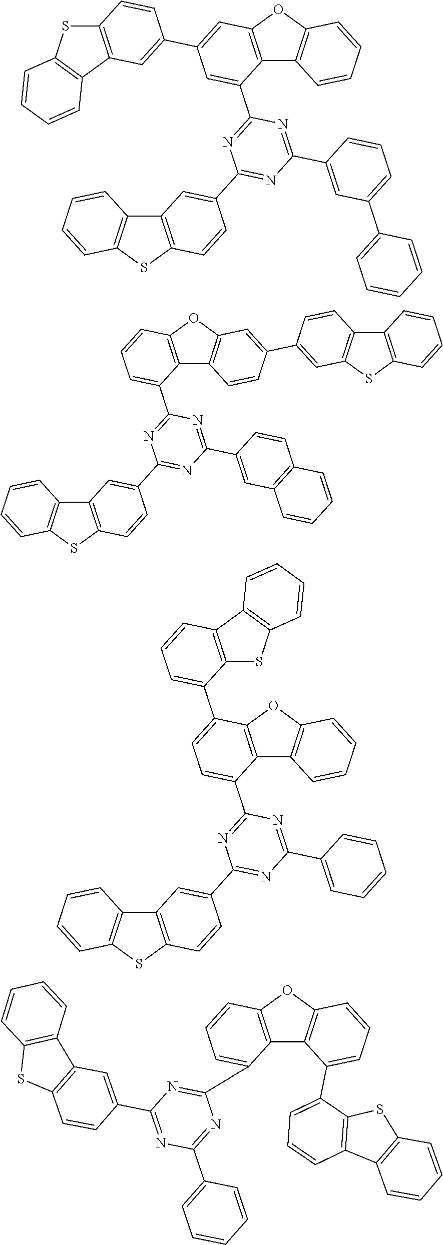

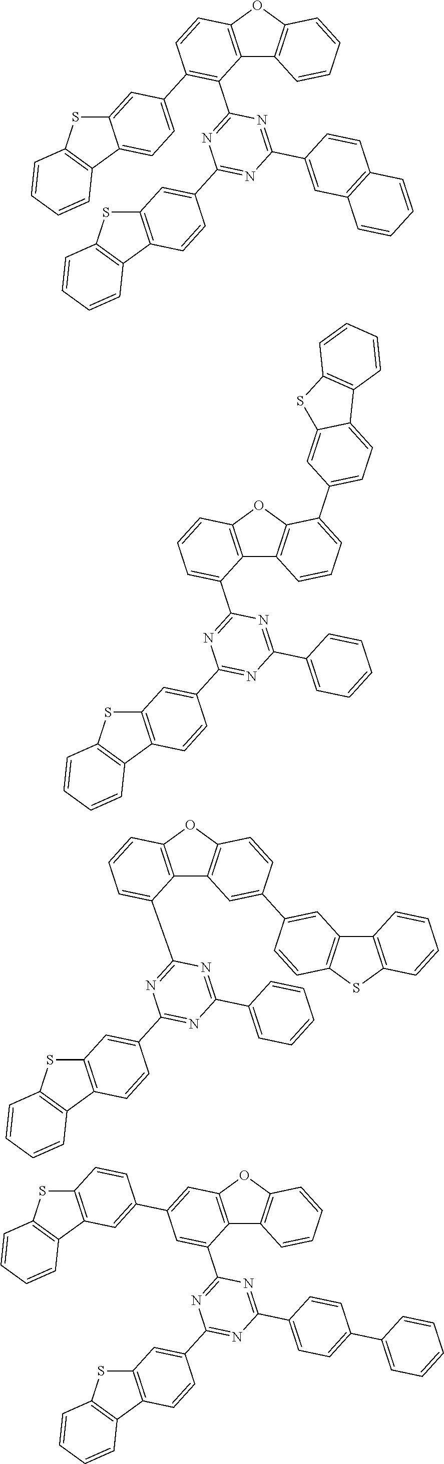

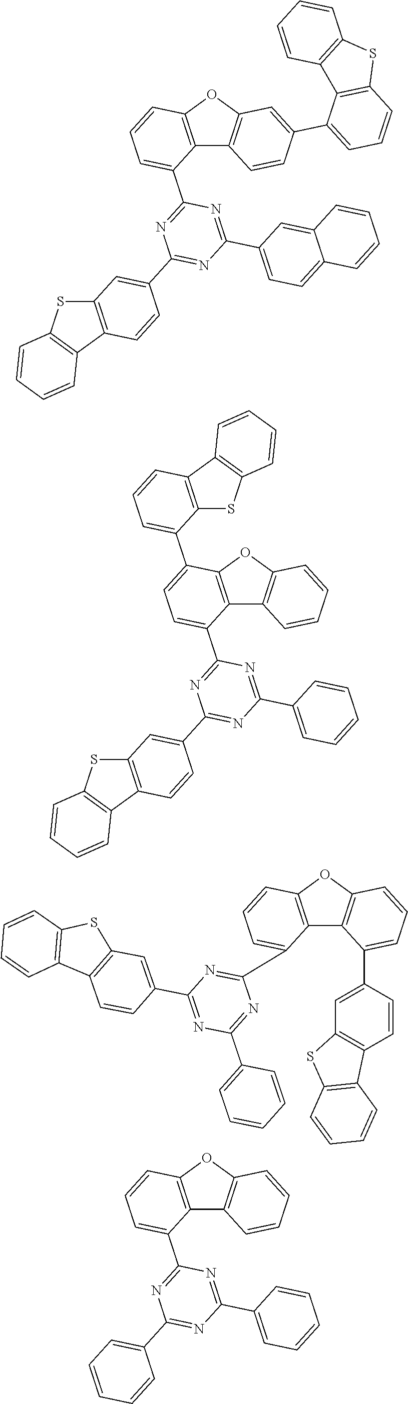

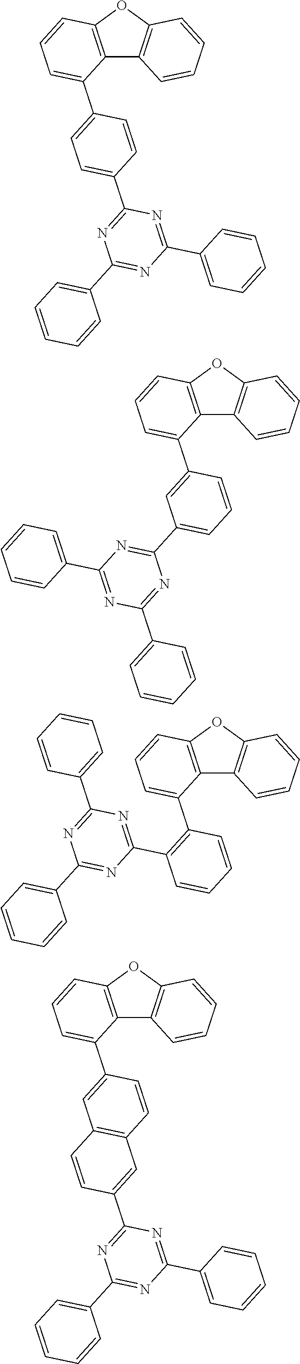

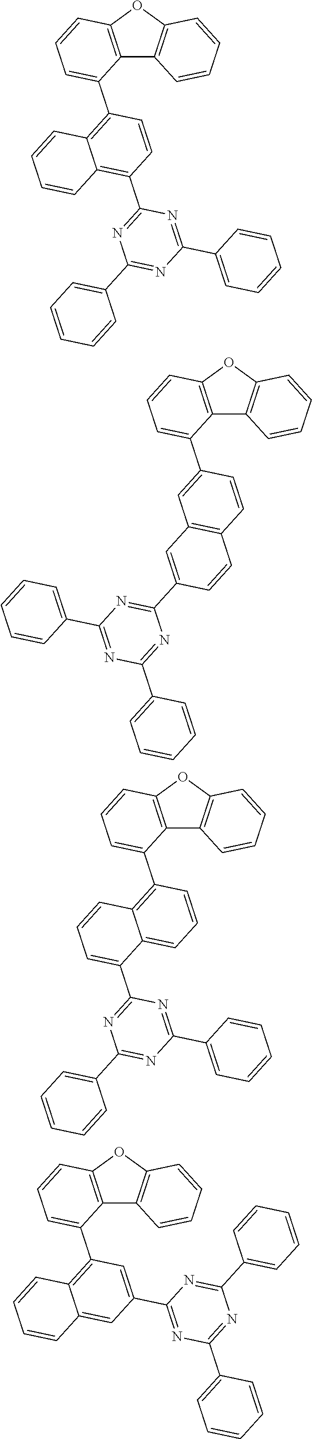

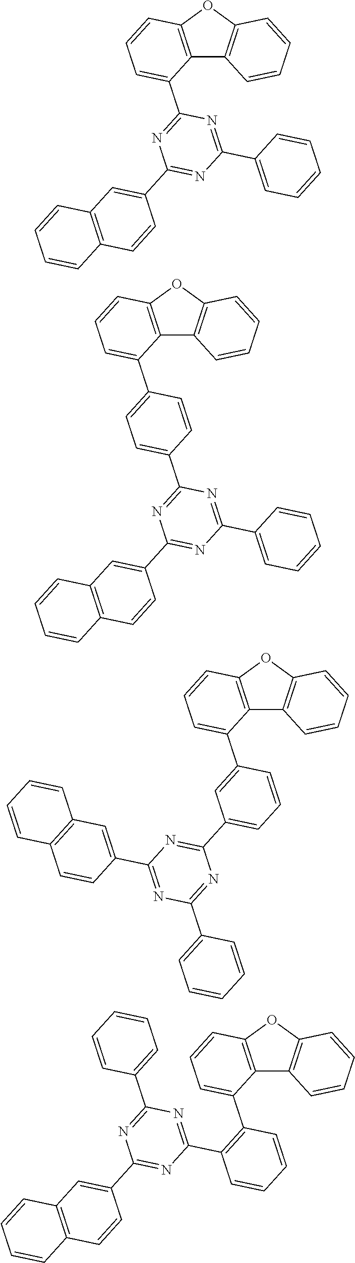

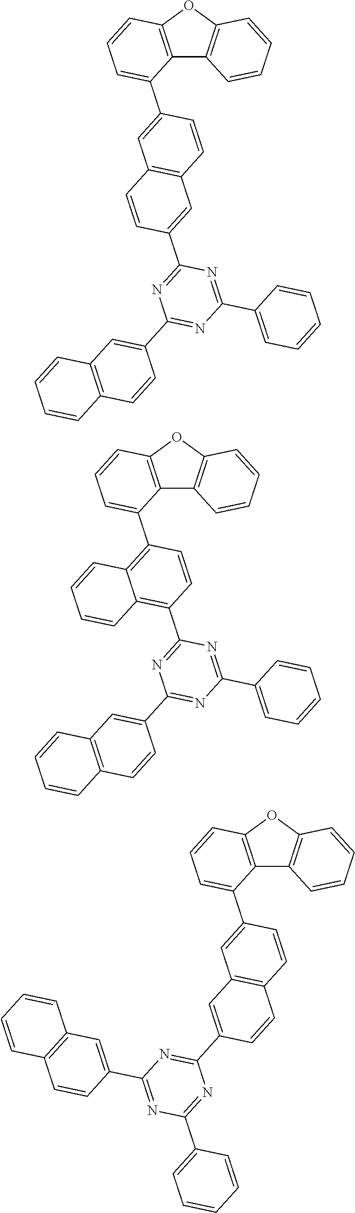 [SMILES: c1ccc(-c2nc(-c3ccc4ccccc4c3)nc(-c3ccc4cc(-c5cccc6oc7ccccc7c56)ccc4c3)n2)cc1 ; c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4ccccc4c3)nc(-c3ccc4ccc(-c5cccc6oc7ccccc7c56)cc4c3)n2)cc1]

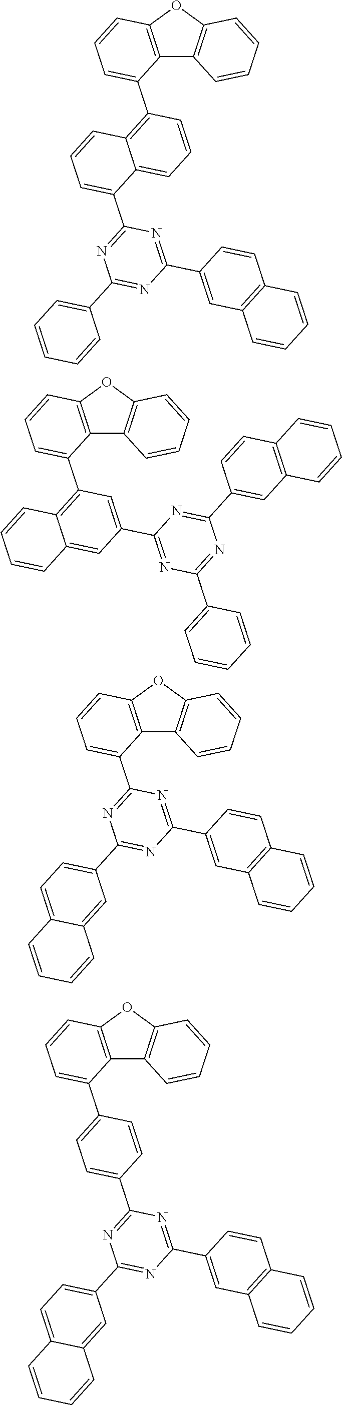

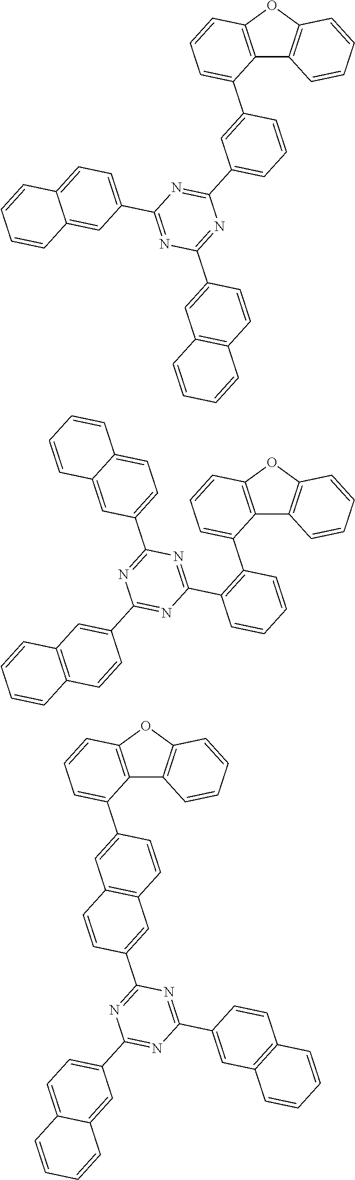 [SMILES: c1cc(-c2nc(-c3ccc4ccccc4c3)nc(-c3ccc4ccccc4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4ccccc4c3)n2)c1 ; c1ccc2cc(-c3nc(-c4ccc5ccccc5c4)nc(-c4ccc5cc(-c6cccc7oc8ccccc8c67)ccc5c4)n3)ccc2c1]

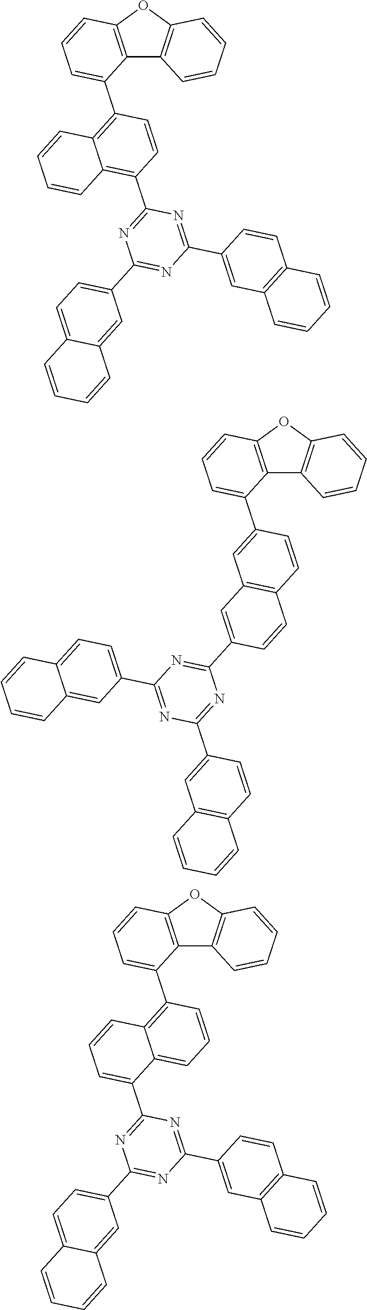

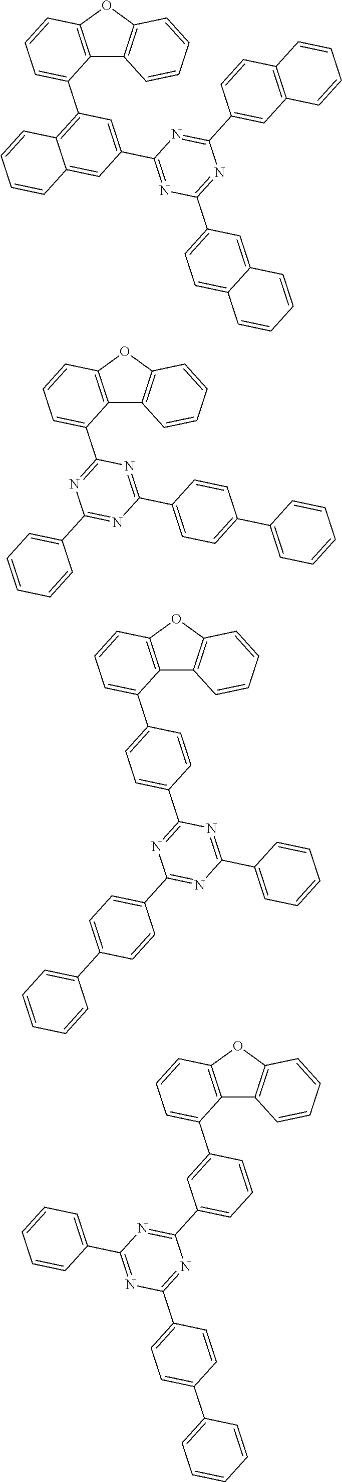 [SMILES: c1ccc2cc(-c3nc(-c4ccc5ccccc5c4)nc(-c4cc(-c5cccc6oc7ccccc7c56)c5ccccc5c4)n3)ccc2c1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1]

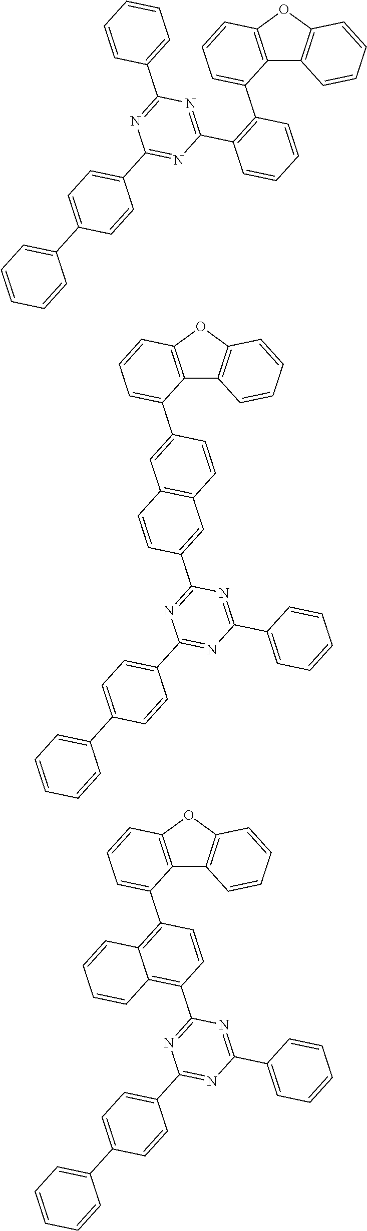 [SMILES: c1ccc(-c2ccc(-c3nc(-c4ccccc4)nc(-c4ccccc4-c4cccc5oc6ccccc6c45)n3)cc2)cc1 ; c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1]

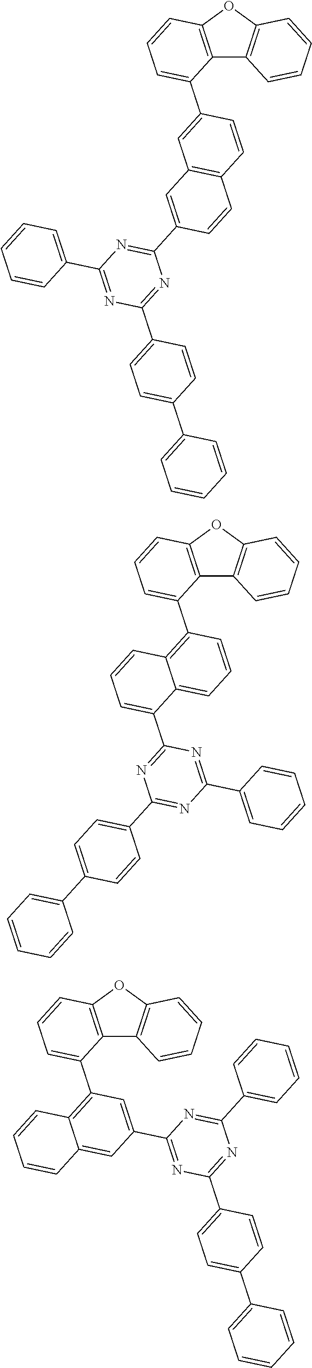

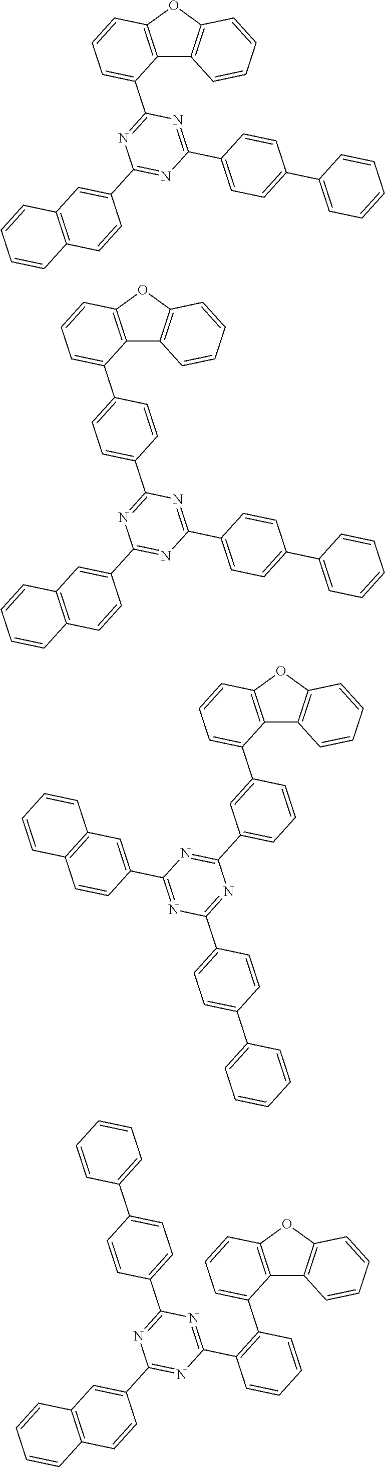 [SMILES: c1ccc(-c2ccc(-c3nc(-c4ccc5ccccc5c4)nc(-c4cccc5oc6ccccc6c45)n3)cc2)cc1 ; c1ccc(-c2ccc(-c3nc(-c4ccc(-c5cccc6oc7ccccc7c56)cc4)nc(-c4ccc5ccccc5c4)n3)cc2)cc1 ; c1ccc(-c2ccc(-c3nc(-c4cccc(-c5cccc6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6ccccc6c45)n3)cc2)cc1]

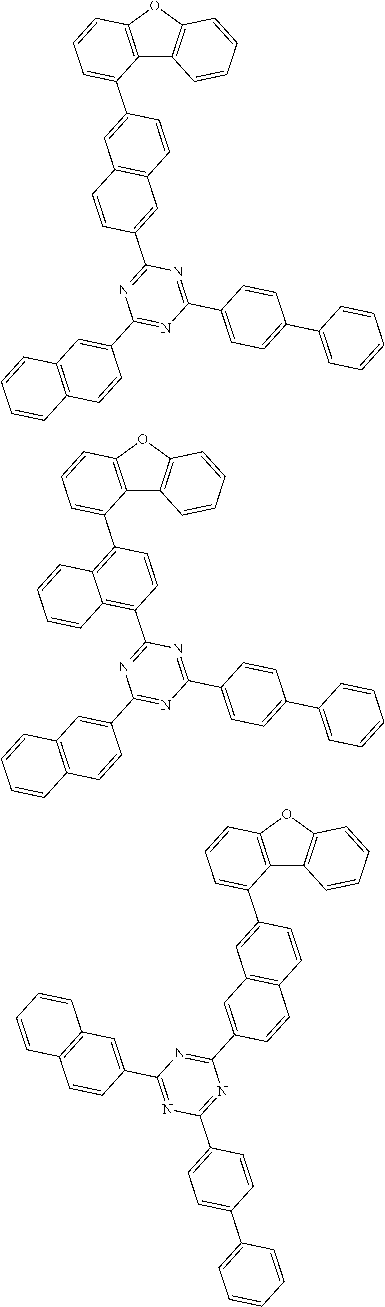 [SMILES: c1ccc(-c2ccc(-c3nc(-c4ccc5ccccc5c4)nc(-c4ccc5cc(-c6cccc7oc8ccccc8c67)ccc5c4)n3)cc2)cc1 ; c1ccc(-c2ccc(-c3nc(-c4ccc5ccccc5c4)nc(-c4ccc(-c5cccc6oc7ccccc7c56)c5ccccc45)n3)cc2)cc1 ; c1ccc(-c2ccc(-c3nc(-c4ccc5ccccc5c4)nc(-c4ccc5ccc(-c6cccc7oc8ccccc8c67)cc5c4)n3)cc2)cc1]

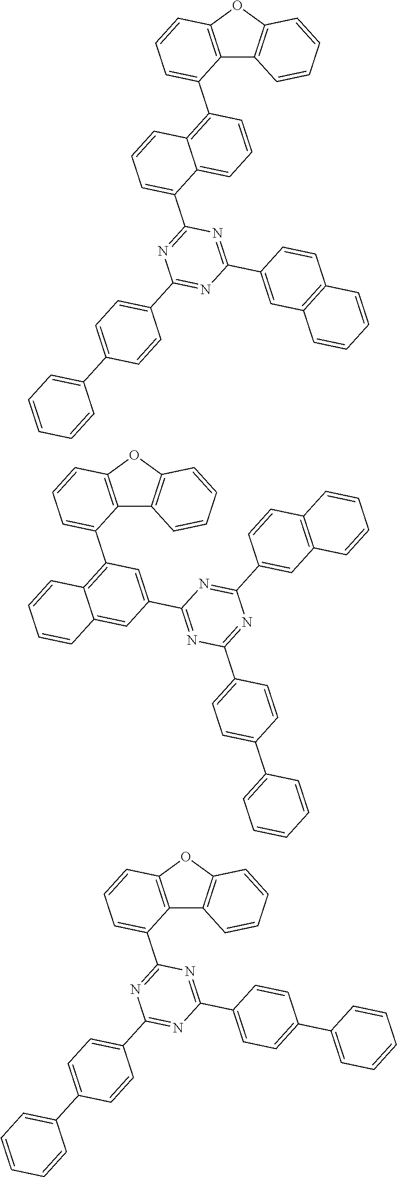

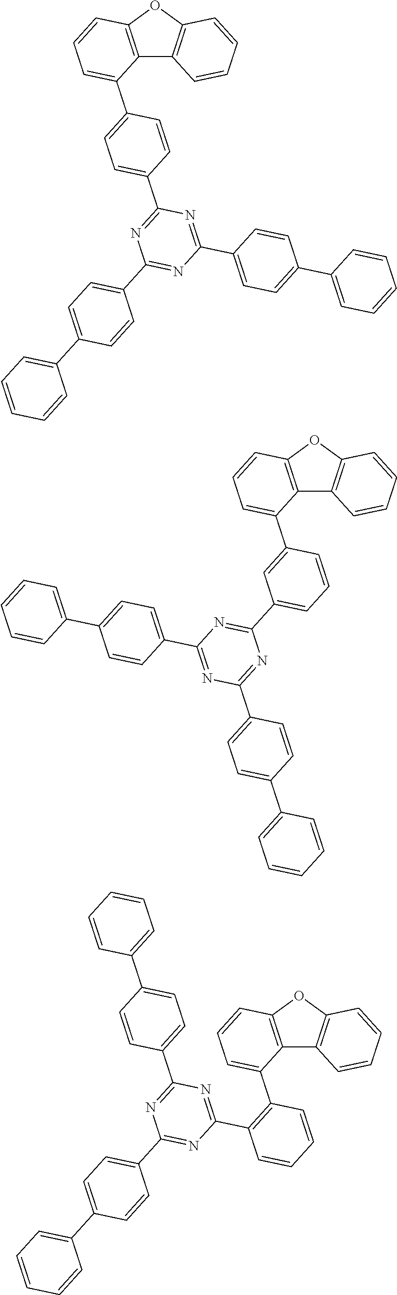 [SMILES: c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4ccccc4-c4cccc5oc6ccccc6c45)n3)cc2)cc1]

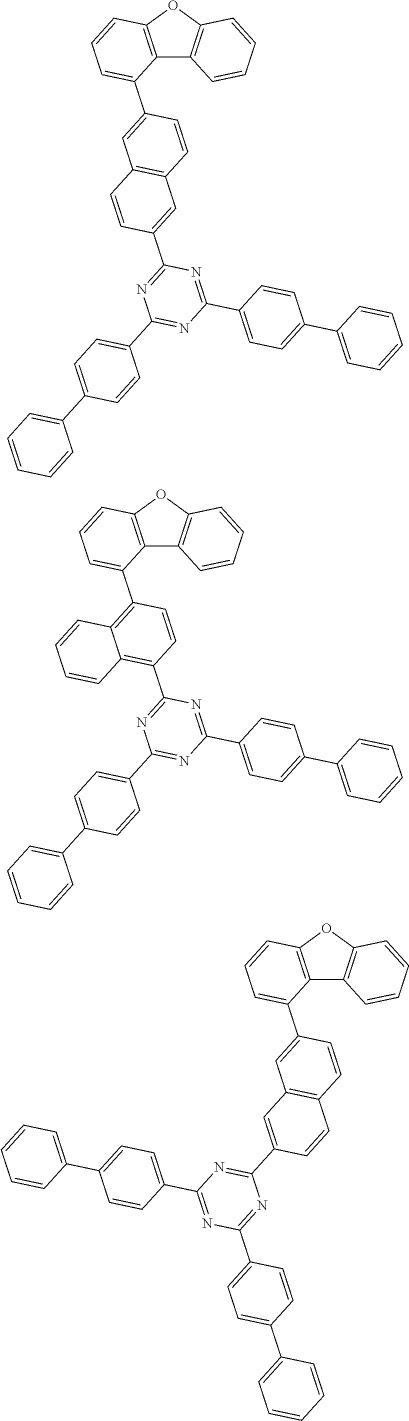 [SMILES: c1ccc(-c2ccc(-c3nc(-c4ccc(-c5ccccc5)cc4)nc(-c4ccc5cc(-c6cccc7oc8ccccc8c67)ccc5c4)n3)cc2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1 ; c1ccc(-c2ccc(-c3nc(-c4ccc(-c5ccccc5)cc4)nc(-c4ccc5ccc(-c6cccc7oc8ccccc8c67)cc5c4)n3)cc2)cc1]

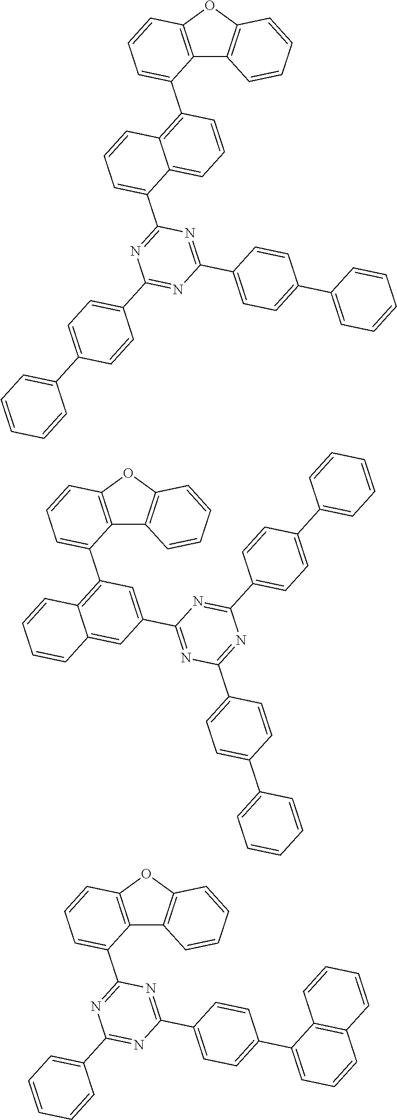 [SMILES: c1ccc(-c2ccc(-c3nc(-c4ccc(-c5ccccc5)cc4)nc(-c4cccc5c(-c6cccc7oc8ccccc8c67)cccc45)n3)cc2)cc1 ; c1ccc(-c2ccc(-c3nc(-c4ccc(-c5ccccc5)cc4)nc(-c4cc(-c5cccc6oc7ccccc7c56)c5ccccc5c4)n3)cc2)cc1 ; c1ccc(-c2nc(-c3ccc(-c4cccc5ccccc45)cc3)nc(-c3cccc4oc5ccccc5c34)n2)cc1]

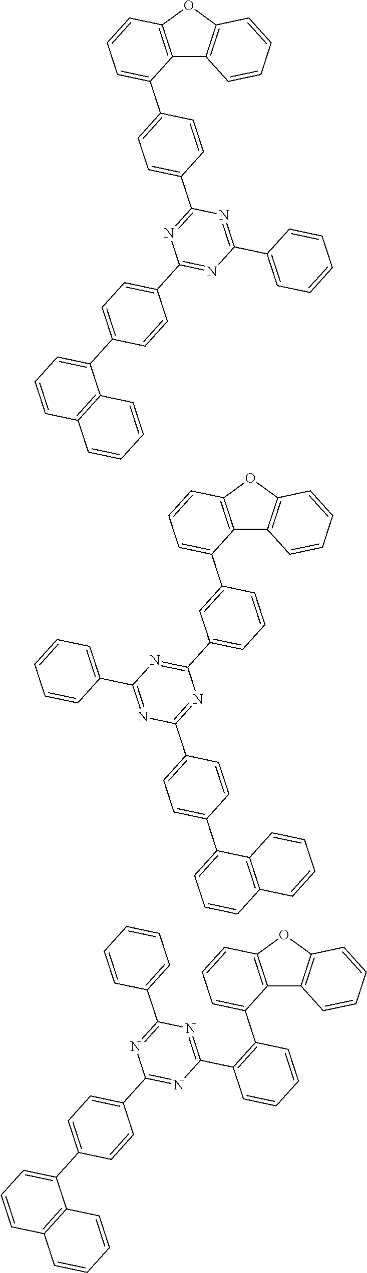

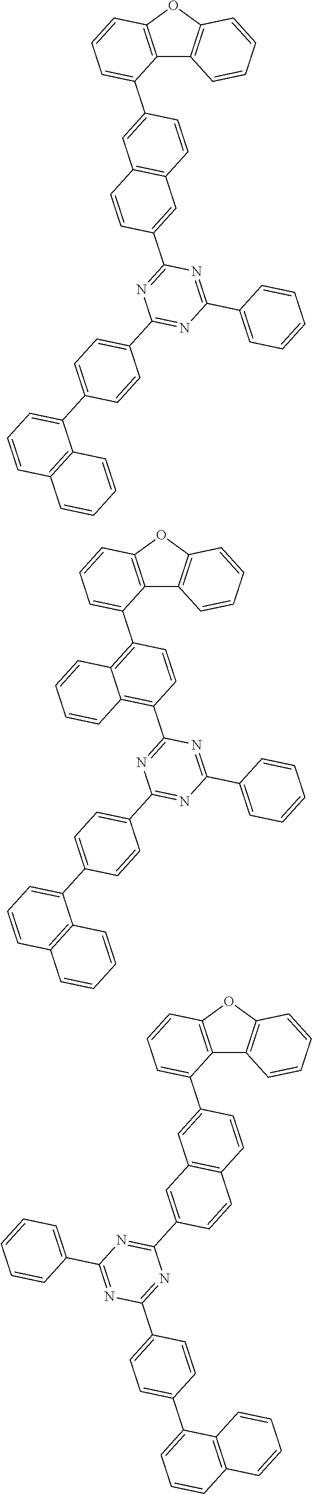

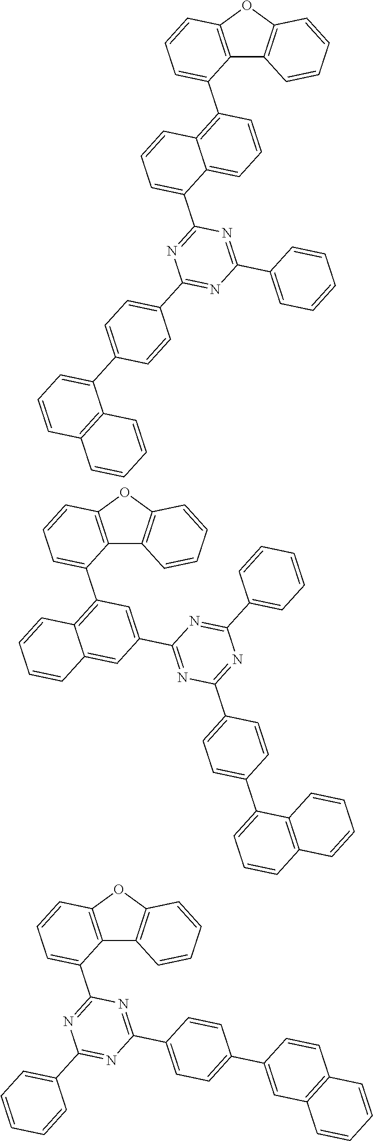

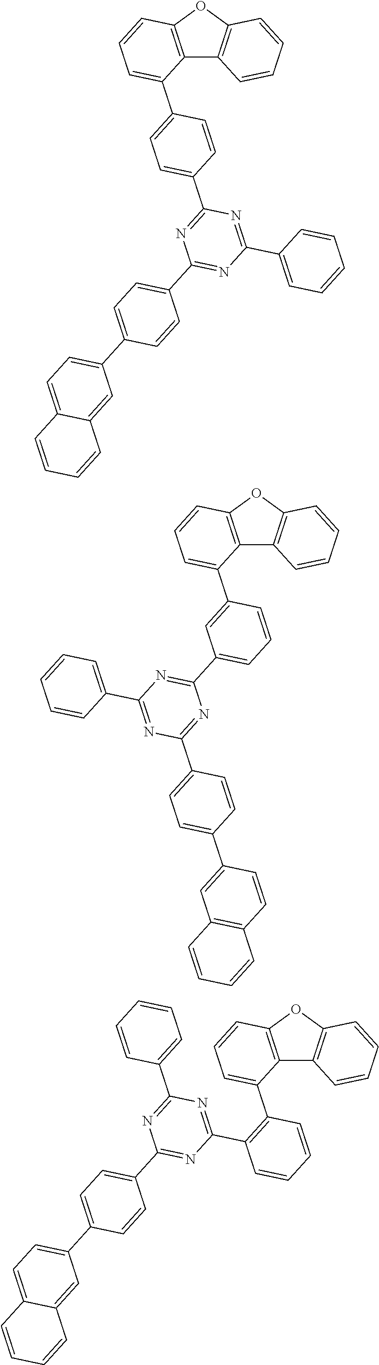

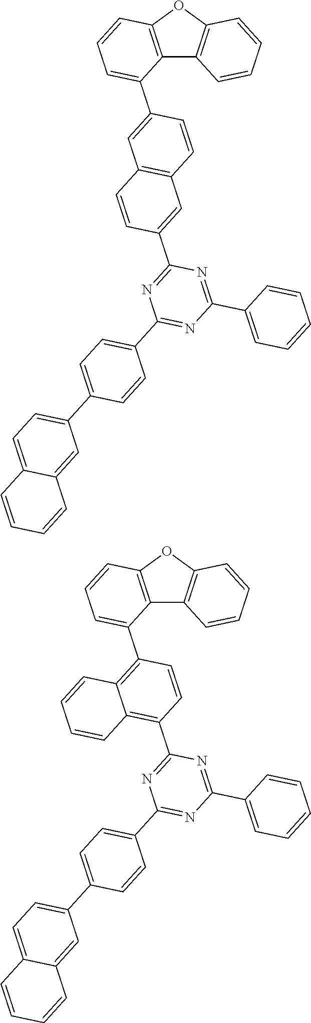 [SMILES: c1ccc(-c2nc(-c3ccc(-c4ccc5ccccc5c4)cc3)nc(-c3ccc4cc(-c5cccc6oc7ccccc7c56)ccc4c3)n2)cc1 ; c1ccc(-c2nc(-c3ccc(-c4ccc5ccccc5c4)cc3)nc(-c3ccc(-c4cccc5oc6ccccc6c45)c4ccccc34)n2)cc1]

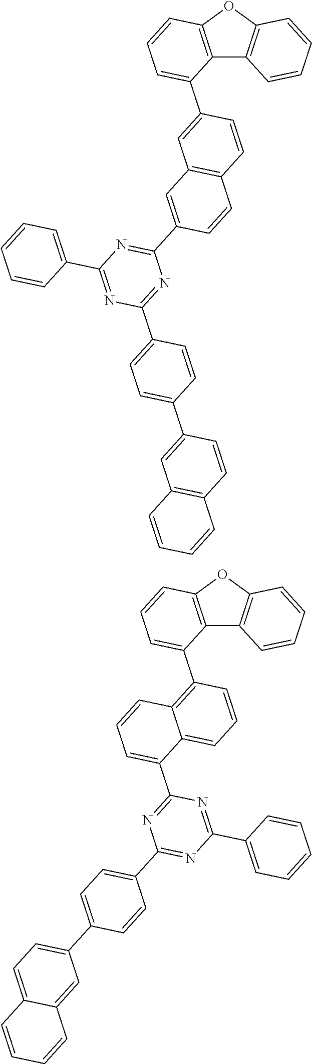

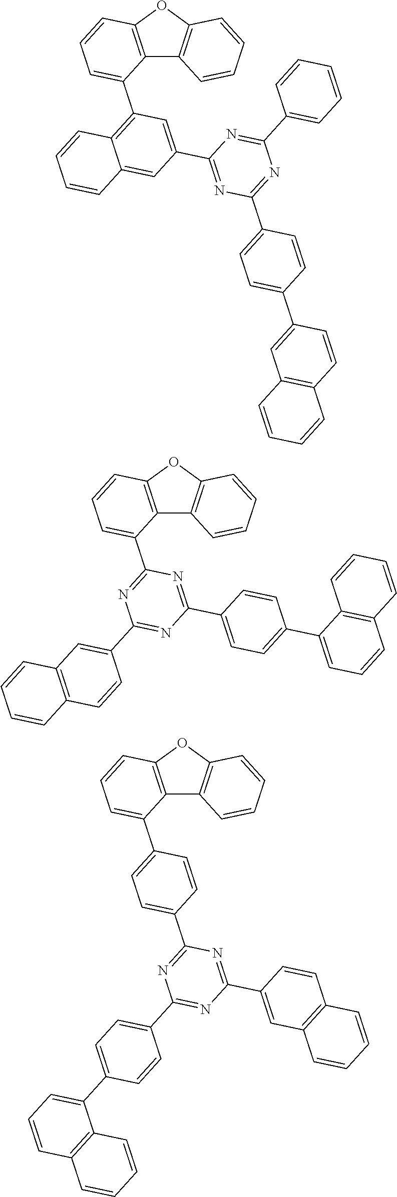 [SMILES: c1ccc(-c2nc(-c3ccc(-c4ccc5ccccc5c4)cc3)nc(-c3cc(-c4cccc5oc6ccccc6c45)c4ccccc4c3)n2)cc1 ; c1ccc2cc(-c3nc(-c4ccc(-c5cccc6ccccc56)cc4)nc(-c4cccc5oc6ccccc6c45)n3)ccc2c1 ; c1ccc2cc(-c3nc(-c4ccc(-c5cccc6ccccc56)cc4)nc(-c4ccc(-c5cccc6oc7ccccc7c56)cc4)n3)ccc2c1]

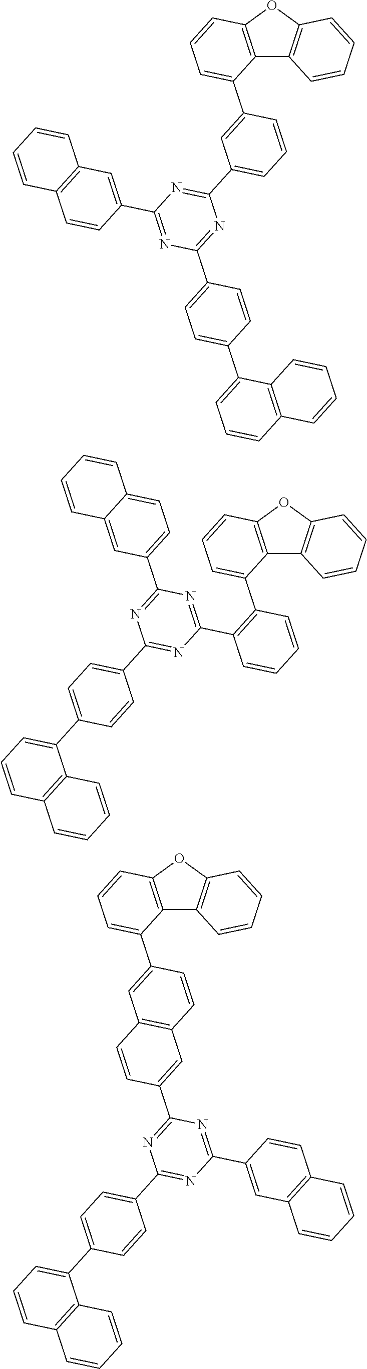

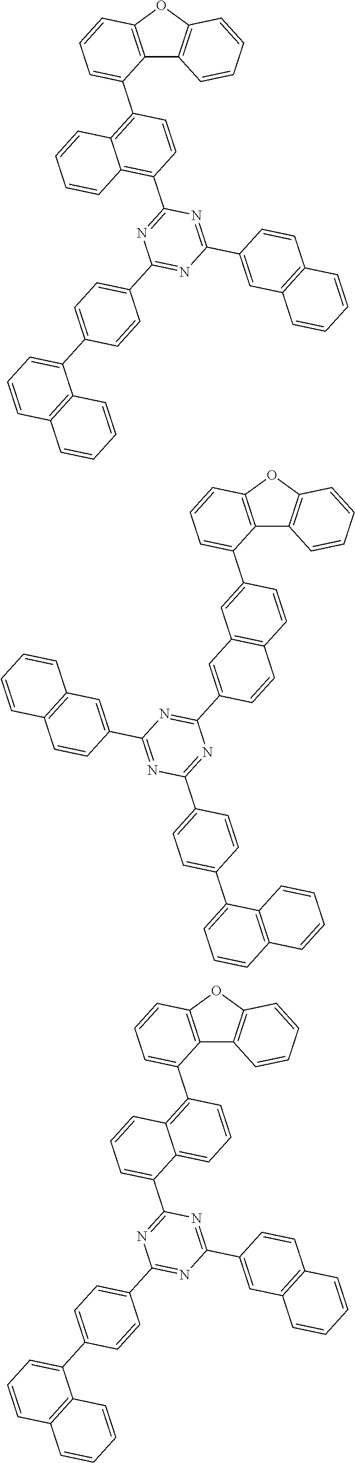

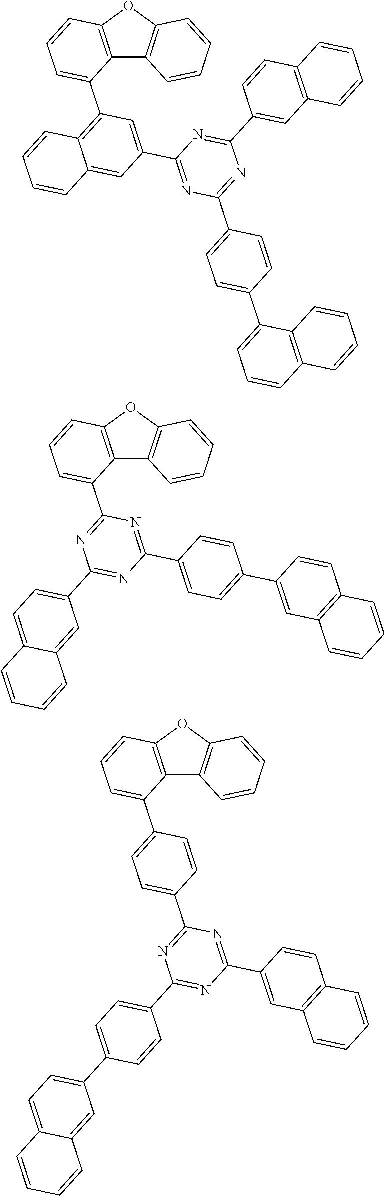 [SMILES: c1ccc2cc(-c3nc(-c4ccc(-c5cccc6ccccc56)cc4)nc(-c4cc(-c5cccc6oc7ccccc7c56)c5ccccc5c4)n3)ccc2c1 ; c1ccc2cc(-c3ccc(-c4nc(-c5ccc6ccccc6c5)nc(-c5cccc6oc7ccccc7c56)n4)cc3)ccc2c1 ; c1ccc2cc(-c3ccc(-c4nc(-c5ccc(-c6cccc7oc8ccccc8c67)cc5)nc(-c5ccc6ccccc6c5)n4)cc3)ccc2c1]

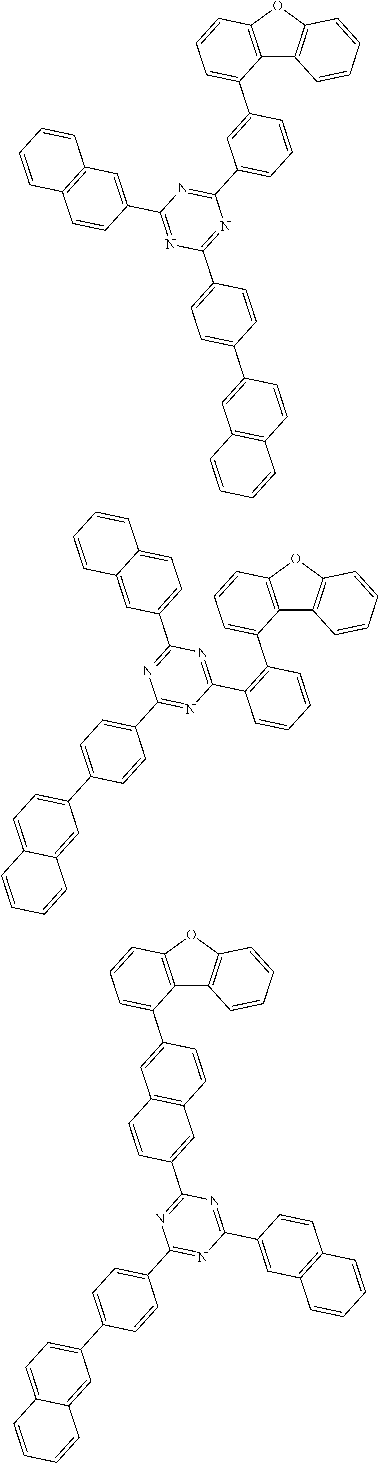 [SMILES: c1cc(-c2nc(-c3ccc(-c4ccc5ccccc5c4)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3ccc4ccccc4c3)n2)c1 ; c1ccc2cc(-c3ccc(-c4nc(-c5ccc6ccccc6c5)nc(-c5ccc6cc(-c7cccc8oc9ccccc9c78)ccc6c5)n4)cc3)ccc2c1]

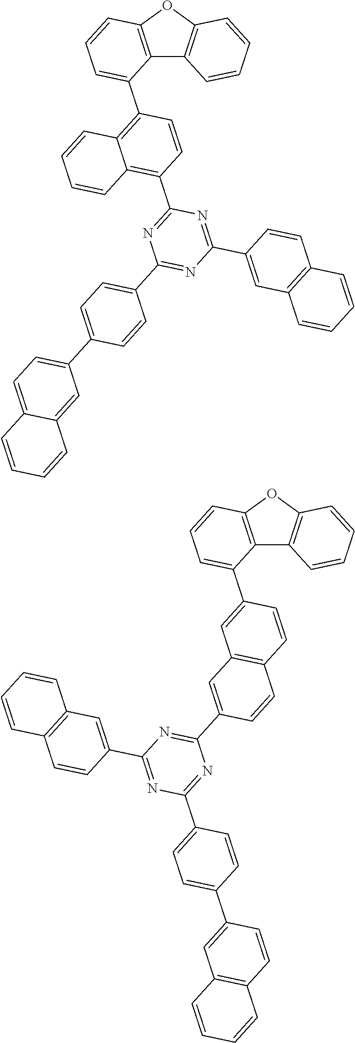 [SMILES: c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1]

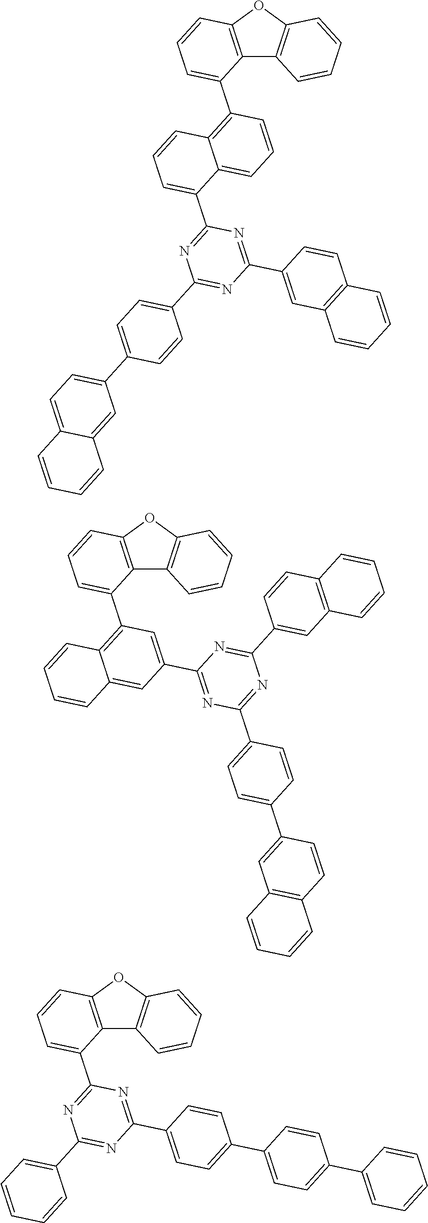

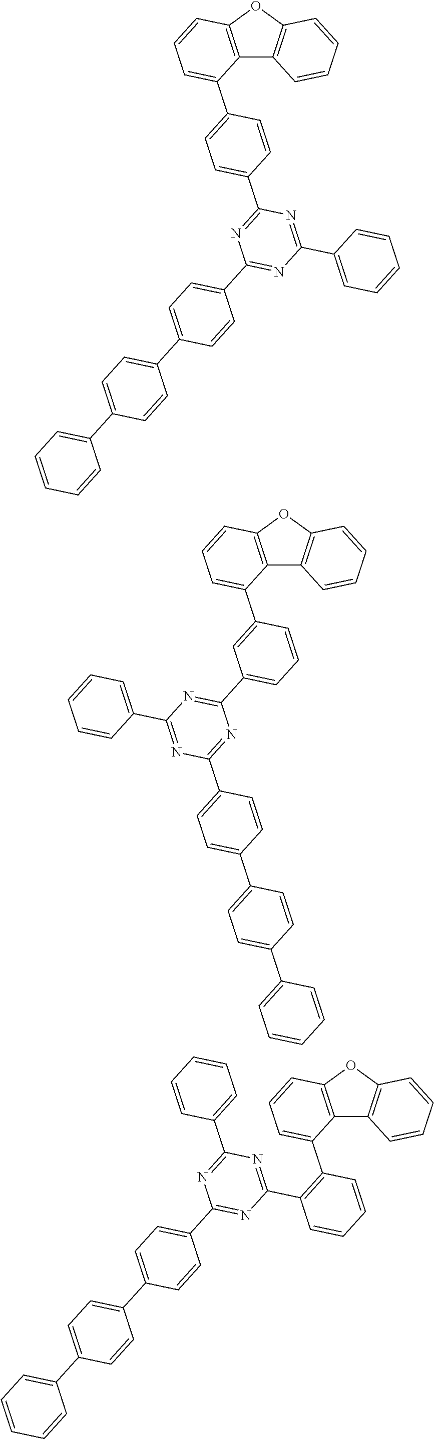

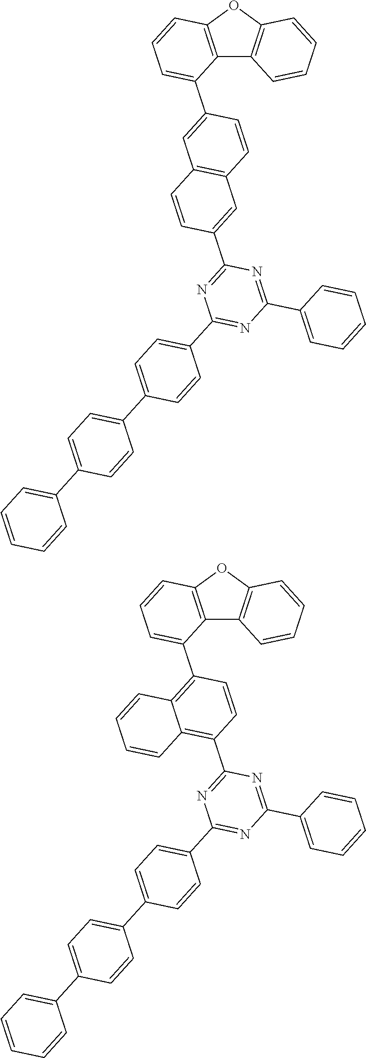 [SMILES: c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

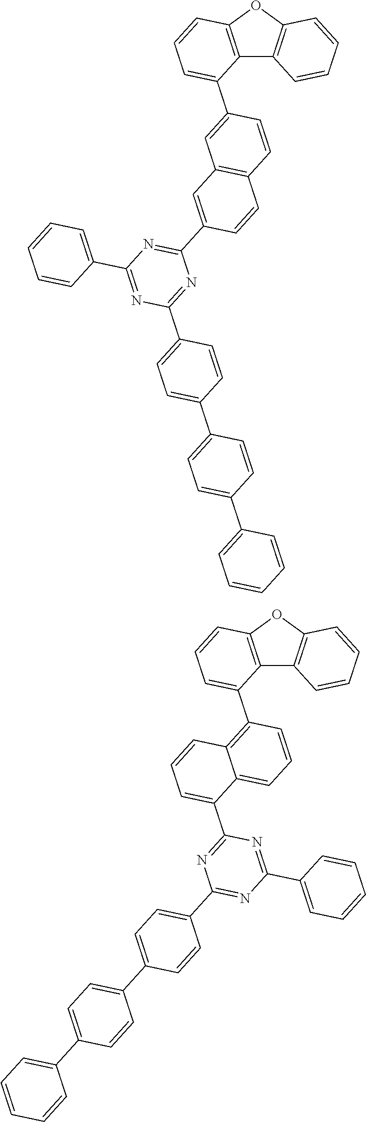

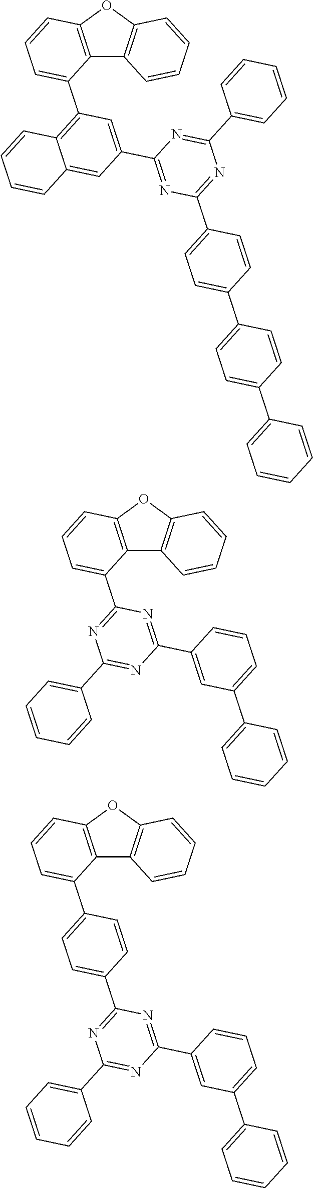 [SMILES: c1ccc(-c2ccc(-c3ccc(-c4nc(-c5ccccc5)nc(-c5cc(-c6cccc7oc8ccccc8c67)c6ccccc6c5)n4)cc3)cc2)cc1 ; c1ccc(-c2cccc(-c3nc(-c4ccccc4)nc(-c4cccc5oc6ccccc6c45)n3)c2)cc1 ; c1ccc(-c2cccc(-c3nc(-c4ccccc4)nc(-c4ccc(-c5cccc6oc7ccccc7c56)cc4)n3)c2)cc1]

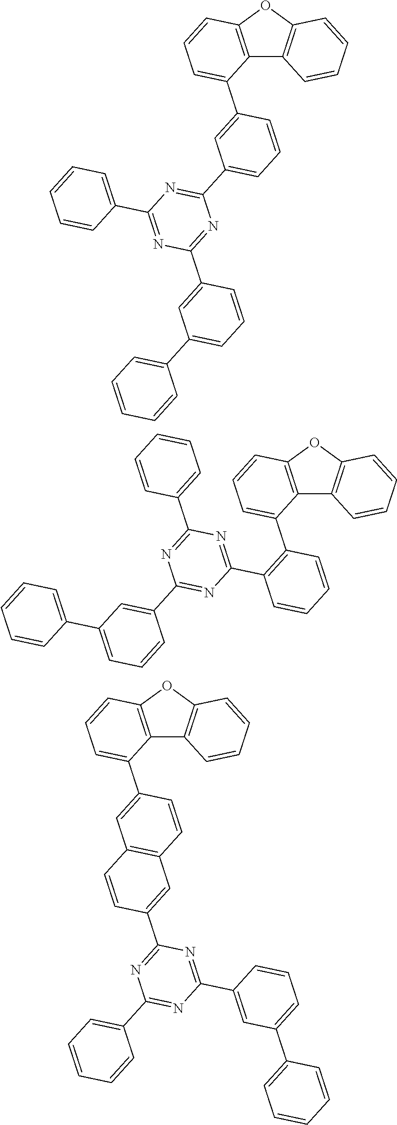

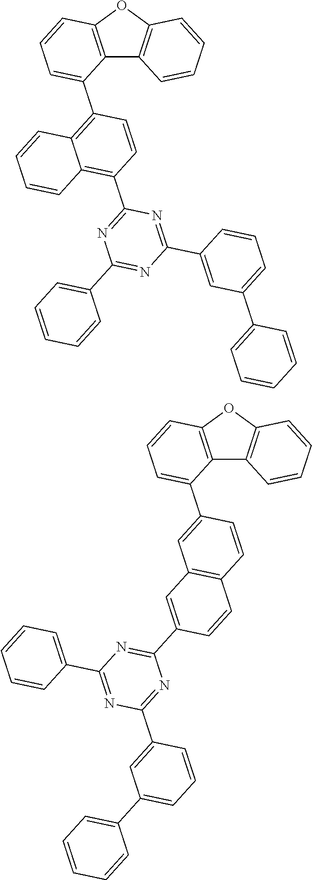

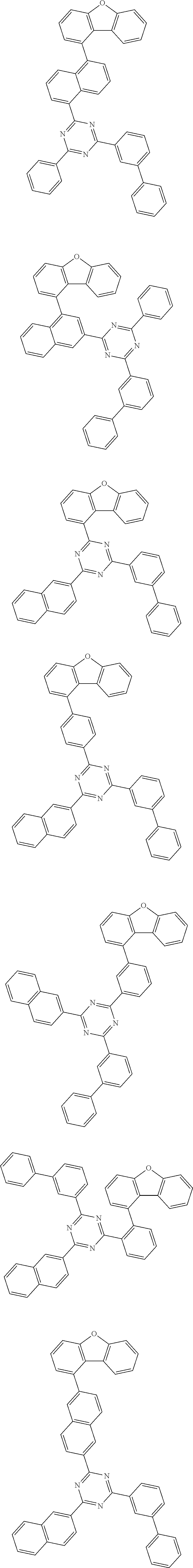

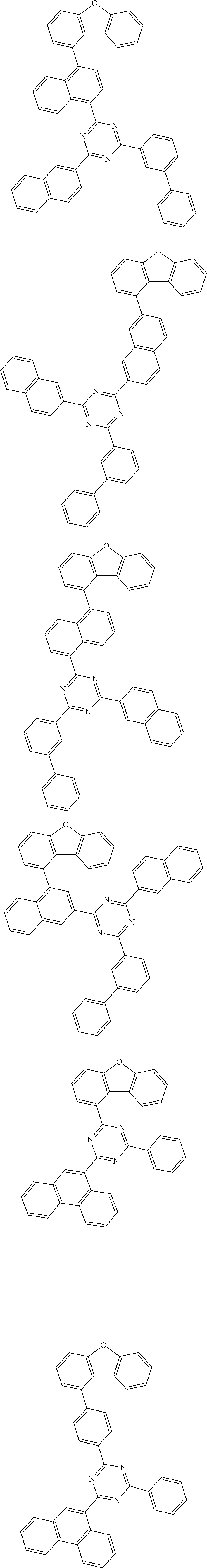

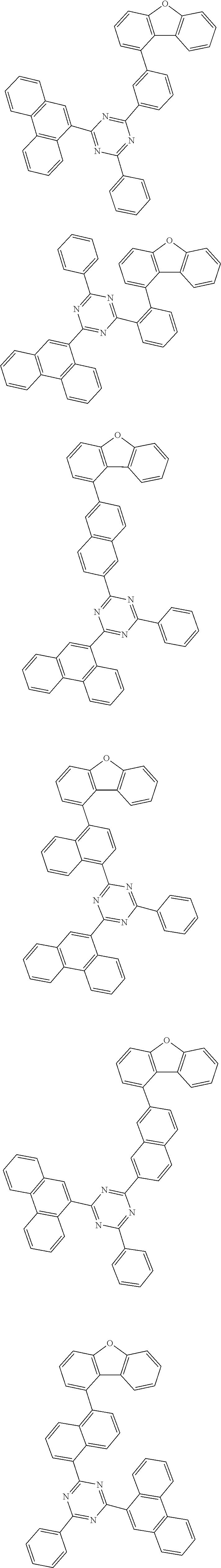 [SMILES: c1ccc(-c2nc(-c3cccc(-c4cccc5oc6ccccc6c45)c3)nc(-c3cc4ccccc4c4ccccc34)n2)cc1 ; c1ccc(-c2nc(-c3ccccc3-c3cccc4oc5ccccc5c34)nc(-c3cc4ccccc4c4ccccc34)n2)cc1 ; c1ccc(-c2nc(-c3ccc4cc(-c5cccc6oc7ccccc7c56)ccc4c3)nc(-c3cc4ccccc4c4ccccc34)n2)cc1 ; c1ccc(-c2nc(-c3ccc(-c4cccc5oc6ccccc6c45)c4ccccc34)nc(-c3cc4ccccc4c4ccccc34)n2)cc1 ; c1ccc(-c2nc(-c3ccc4ccc(-c5cccc6oc7ccccc7c56)cc4c3)nc(-c3cc4ccccc4c4ccccc34)n2)cc1 ; c1ccc(-c2nc(-c3cccc4c(-c5cccc6oc7ccccc7c56)cccc34)nc(-c3cc4ccccc4c4ccccc34)n2)cc1]

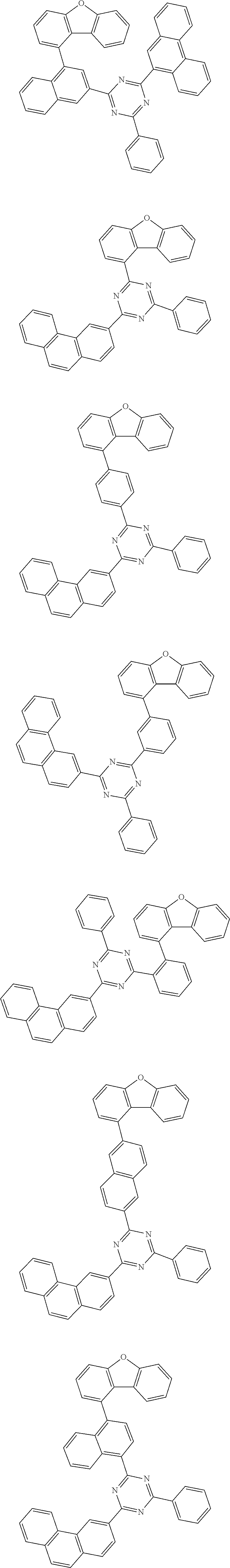

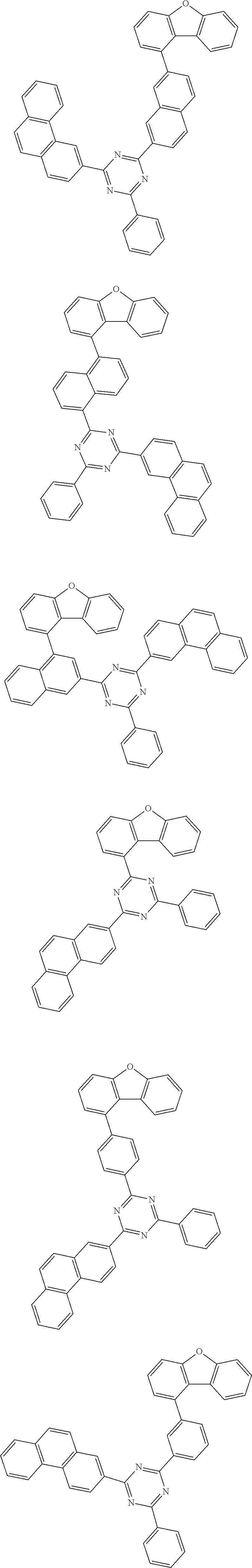 [SMILES: c1ccc(-c2nc(-c3ccc4ccc(-c5cccc6oc7ccccc7c56)cc4c3)nc(-c3ccc4ccc5ccccc5c4c3)n2)cc1 ; c1ccc(-c2nc(-c3ccc4ccc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4ccc5ccccc5c4c3)n2)cc1 ; c1ccc(-c2nc(-c3ccc4c(ccc5ccccc54)c3)nc(-c3cccc4oc5ccccc5c34)n2)cc1 ; c1ccc(-c2nc(-c3ccc(-c4cccc5oc6ccccc6c45)cc3)nc(-c3ccc4c(ccc5ccccc54)c3)n2)cc1 ; c1ccc(-c2nc(-c3cccc(-c4cccc5oc6ccccc6c45)c3)nc(-c3ccc4c(ccc5ccccc54)c3)n2)cc1]

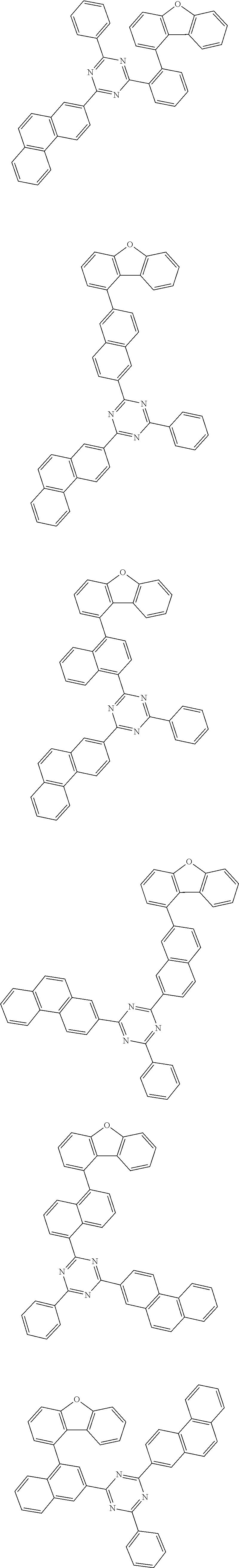

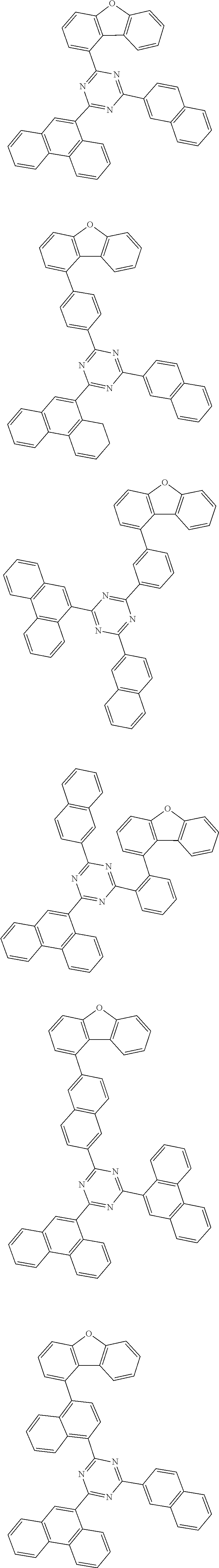

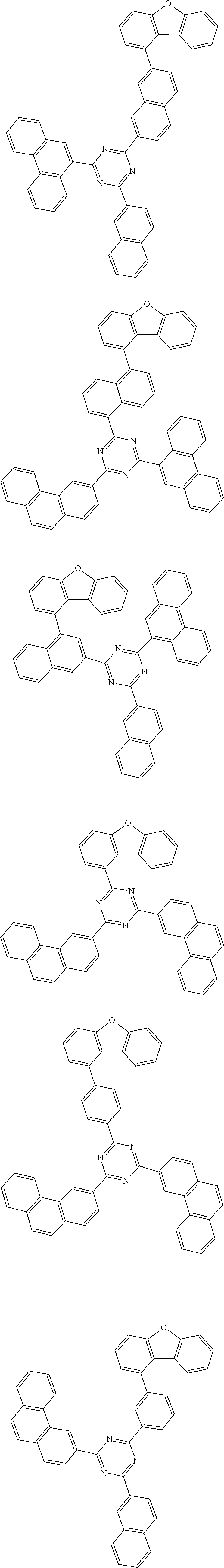

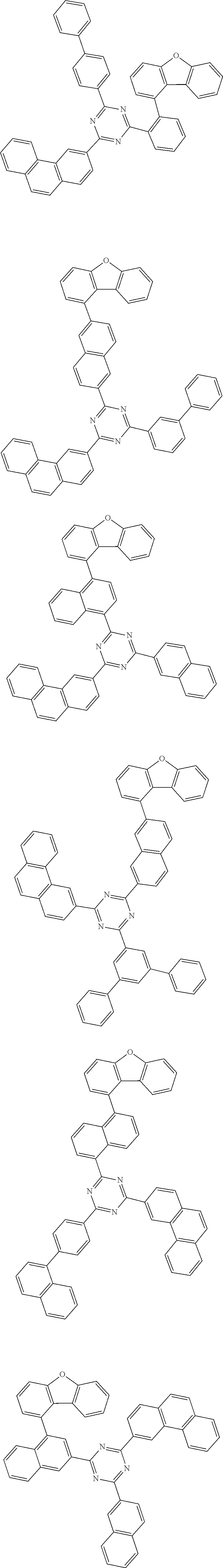

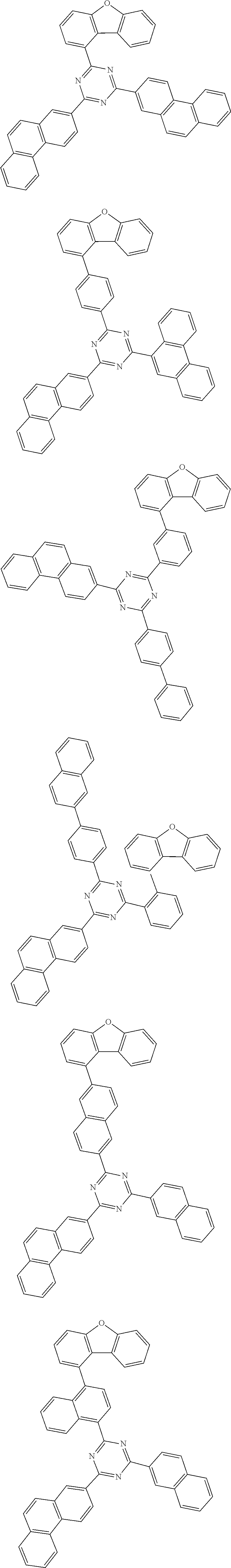

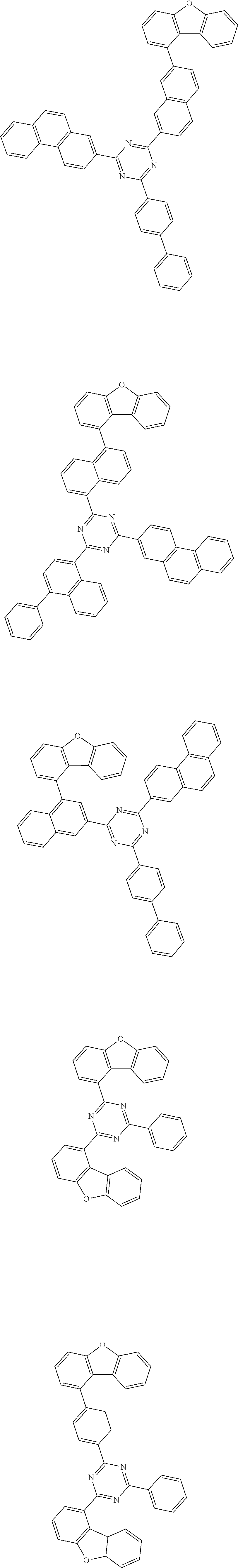 [SMILES: c1ccc(-c2ccc(-c3nc(-c4ccc5ccc(-c6cccc7oc8ccccc8c67)cc5c4)nc(-c4ccc5c(ccc6ccccc65)c4)n3)cc2)cc1 ; c1ccc(-c2ccc(-c3nc(-c4ccc5c(ccc6ccccc65)c4)nc(-c4cccc5c(-c6cccc7oc8ccccc8c67)cccc45)n3)c3ccccc23)cc1 ; c1ccc(-c2ccc(-c3nc(-c4cc(-c5cccc6oc7ccccc7c56)c5ccccc5c4)nc(-c4ccc5c(ccc6ccccc65)c4)n3)cc2)cc1 ; c1ccc(-c2nc(-c3cccc4oc5ccccc5c34)nc(-c3cccc4oc5ccccc5c34)n2)cc1 ; C1=CC2Oc3cccc(-c4nc(C5=CC=C(c6cccc7oc8ccccc8c67)CC5)nc(-c5ccccc5)n4)c3C2C=C1]

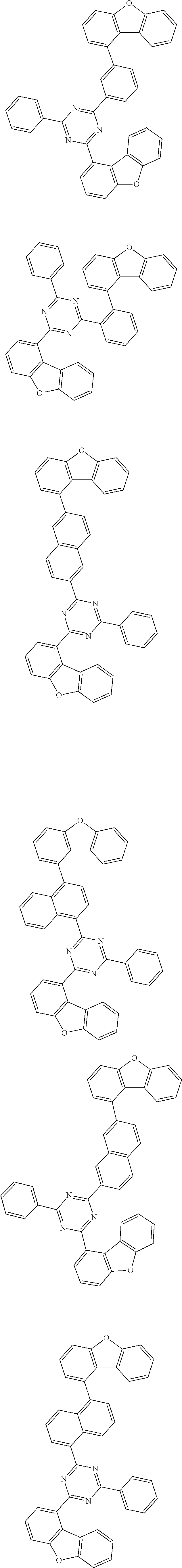

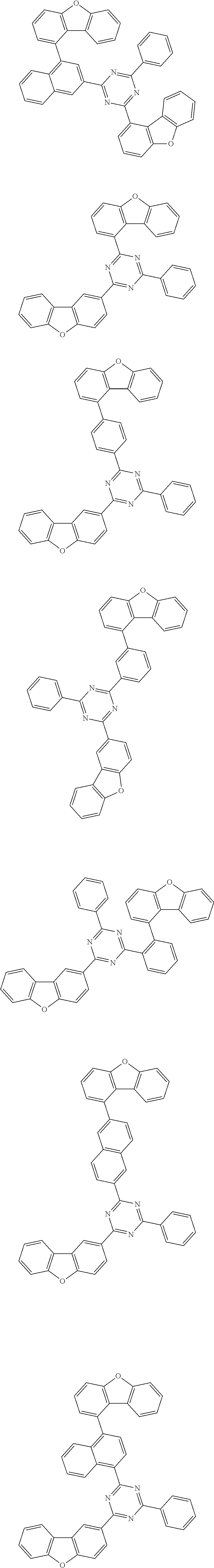

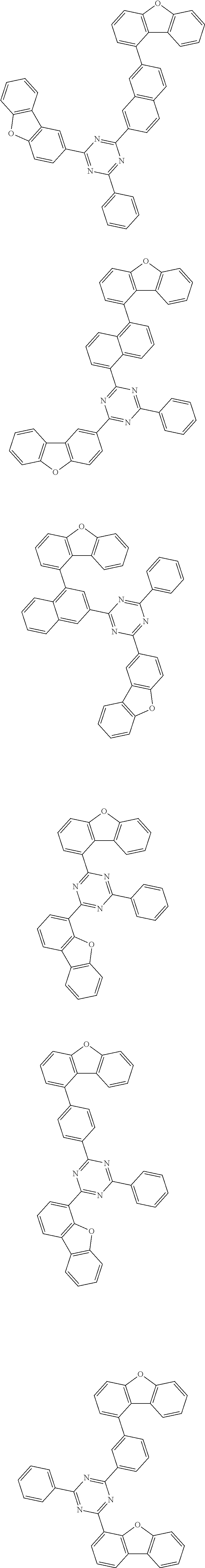 [SMILES: c1ccc(-c2nc(-c3ccc4ccc(-c5cccc6oc7ccccc7c56)cc4c3)nc(-c3ccc4oc5ccccc5c4c3)n2)cc1 ; c1ccc(-c2nc(-c3ccc4oc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4oc5ccccc5c4c3)n2)cc1 ; c1ccc(-c2nc(-c3cccc4c3oc3ccccc34)nc(-c3cccc4oc5ccccc5c34)n2)cc1 ; c1ccc(-c2nc(-c3ccc(-c4cccc5oc6ccccc6c45)cc3)nc(-c3cccc4c3oc3ccccc34)n2)cc1 ; c1ccc(-c2nc(-c3cccc(-c4cccc5oc6ccccc6c45)c3)nc(-c3cccc4c3oc3ccccc34)n2)cc1]

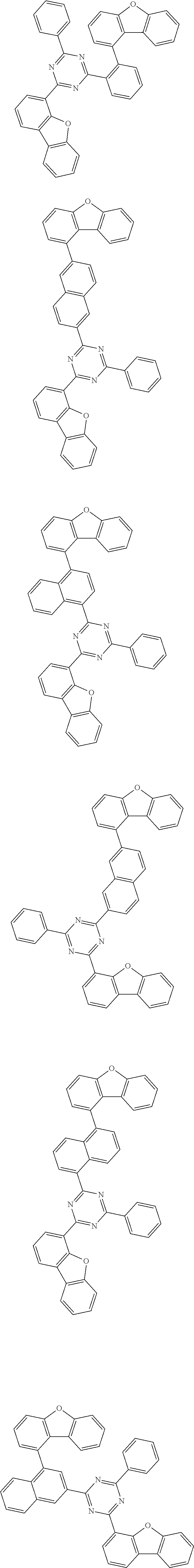 [SMILES: c1ccc(-c2nc(-c3ccccc3-c3cccc4oc5ccccc5c34)nc(-c3cccc4c3oc3ccccc34)n2)cc1 ; c1ccc(-c2nc(-c3ccc4cc(-c5cccc6oc7ccccc7c56)ccc4c3)nc(-c3cccc4c3oc3ccccc34)n2)cc1 ; c1ccc(-c2nc(-c3ccc(-c4cccc5oc6ccccc6c45)c4ccccc34)nc(-c3cccc4c3oc3ccccc34)n2)cc1 ; c1ccc(-c2nc(-c3ccc4ccc(-c5cccc6oc7ccccc7c56)cc4c3)nc(-c3cccc4c3oc3ccccc34)n2)cc1 ; c1ccc(-c2nc(-c3cccc4c(-c5cccc6oc7ccccc7c56)cccc34)nc(-c3cccc4c3oc3ccccc34)n2)cc1 ; c1ccc(-c2nc(-c3cc(-c4cccc5oc6ccccc6c45)c4ccccc4c3)nc(-c3cccc4c3oc3ccccc34)n2)cc1]

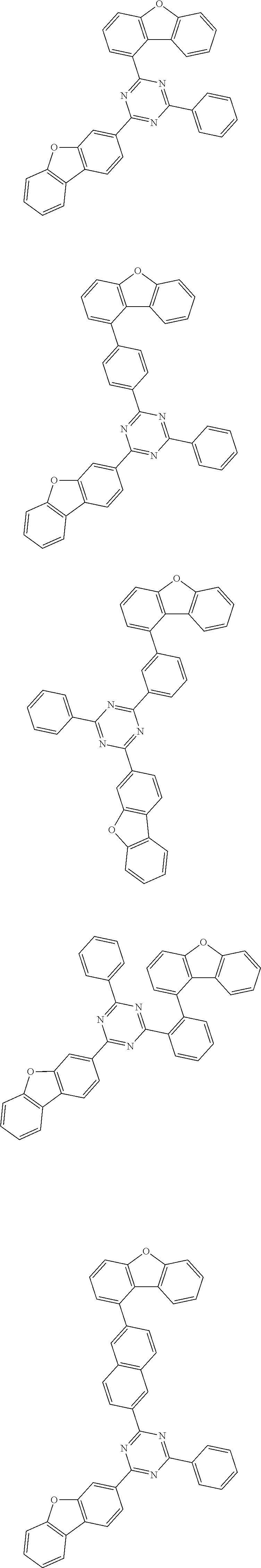 [SMILES: c1ccc(-c2nc(-c3ccc4c(c3)oc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)oc3ccccc34)n2)cc1 ; c1ccc(-c2nc(-c3cccc(-c4cccc5oc6ccccc6c45)c3)nc(-c3ccc4c(c3)oc3ccccc34)n2)cc1 ; c1ccc(-c2nc(-c3ccc4c(c3)oc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)oc3ccccc34)n2)cc1]

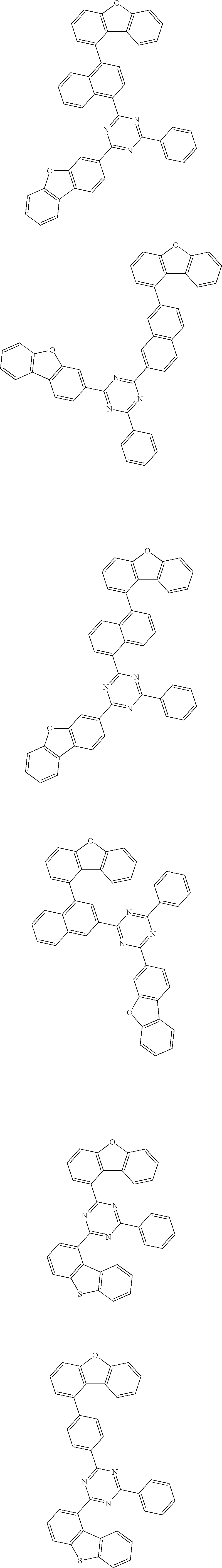 [SMILES: c1ccc(-c2nc(-c3ccc4c(c3)oc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)oc3ccccc34)n2)cc1 ; c1ccc(-c2nc(-c3ccc4c(c3)oc3ccccc34)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(c3)oc3ccccc34)n2)cc1 ; c1ccc(-c2nc(-c3cccc4oc5ccccc5c34)nc(-c3cccc4sc5ccccc5c34)n2)cc1 ; c1ccc(-c2nc(-c3ccc(-c4cccc5oc6ccccc6c45)cc3)nc(-c3cccc4sc5ccccc5c34)n2)cc1]

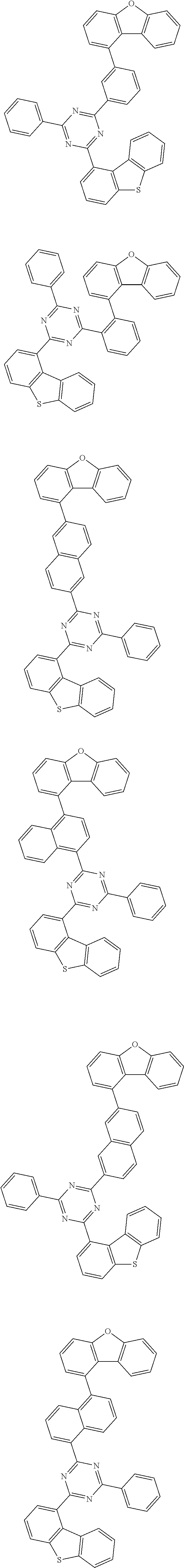 [SMILES: c1ccc(-c2nc(-c3cccc(-c4cccc5oc6ccccc6c45)c3)nc(-c3cccc4sc5ccccc5c34)n2)cc1 ; c1ccc(-c2nc(-c3ccccc3-c3cccc4oc5ccccc5c34)nc(-c3cccc4sc5ccccc5c34)n2)cc1 ; c1ccc(-c2nc(-c3ccc4cc(-c5cccc6oc7ccccc7c56)ccc4c3)nc(-c3cccc4sc5ccccc5c34)n2)cc1 ; c1ccc(-c2nc(-c3ccc(-c4cccc5oc6ccccc6c45)c4ccccc34)nc(-c3cccc4sc5ccccc5c34)n2)cc1 ; c1ccc(-c2nc(-c3ccc4ccc(-c5cccc6oc7ccccc7c56)cc4c3)nc(-c3cccc4sc5ccccc5c34)n2)cc1 ; c1ccc(-c2nc(-c3cccc4c(-c5cccc6oc7ccccc7c56)cccc34)nc(-c3cccc4sc5ccccc5c34)n2)cc1]

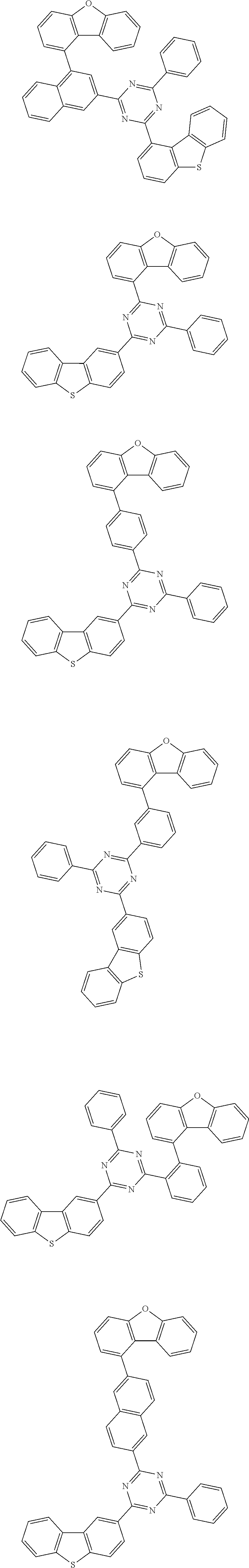 [SMILES: c1ccc(-c2nc(-c3cc(-c4cccc5oc6ccccc6c45)c4ccccc4c3)nc(-c3cccc4sc5ccccc5c34)n2)cc1 ; c1ccc(-c2nc(-c3ccc4sc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4sc5ccccc5c4c3)n2)cc1 ; c1ccc(-c2nc(-c3cccc(-c4cccc5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4sc5ccccc5c4c3)n2)cc1]

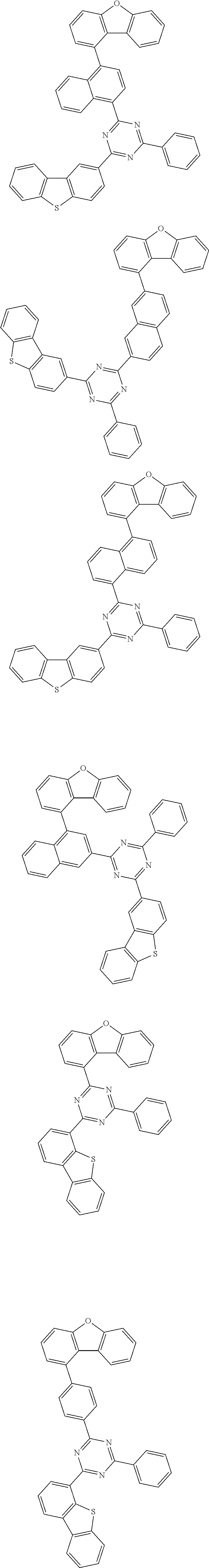

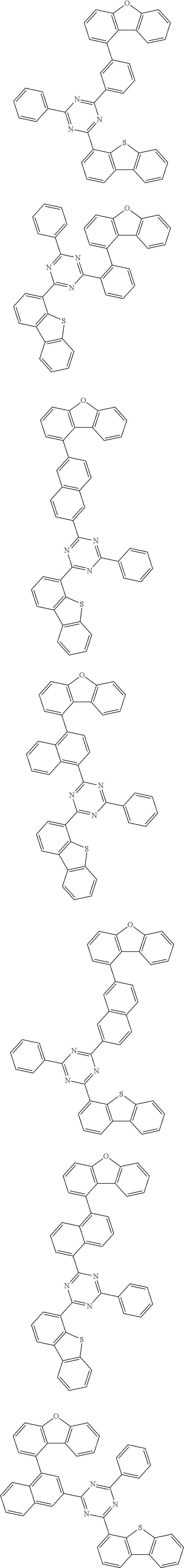

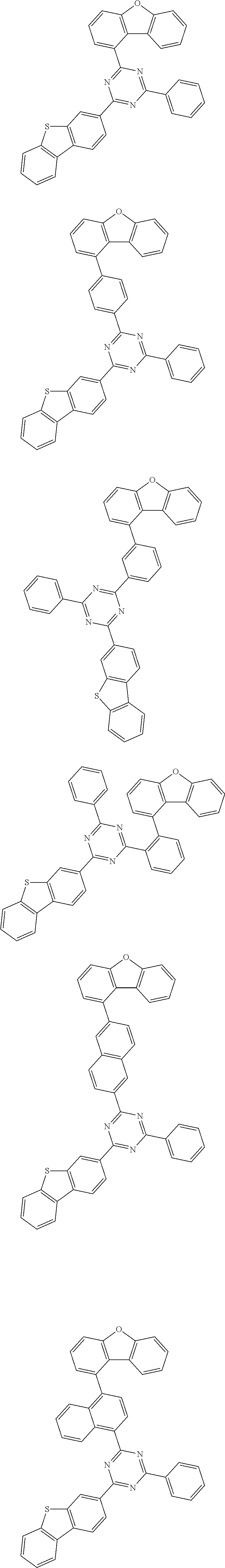 [SMILES: c1ccc(-c2nc(-c3ccc4c(c3)sc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)sc3ccccc34)n2)cc1 ; c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)sc3ccccc34)n2)cc1 ; c1ccc(-c2nc(-c3ccc4c(c3)sc3ccccc34)nc(-c3ccc(-c4cccc5oc6ccccc6c45)c4ccccc34)n2)cc1]

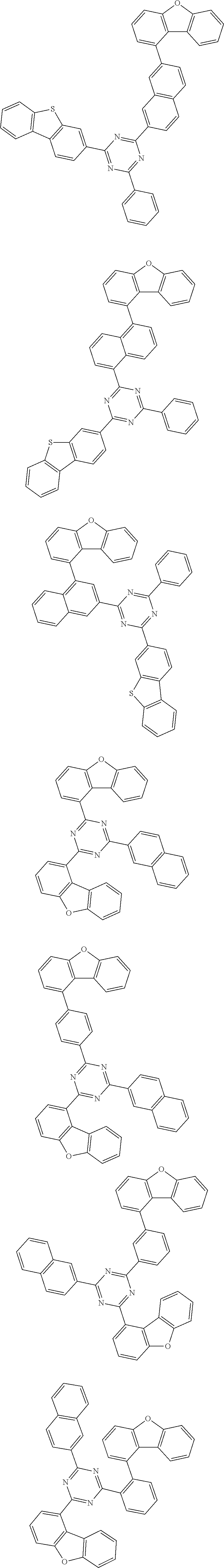

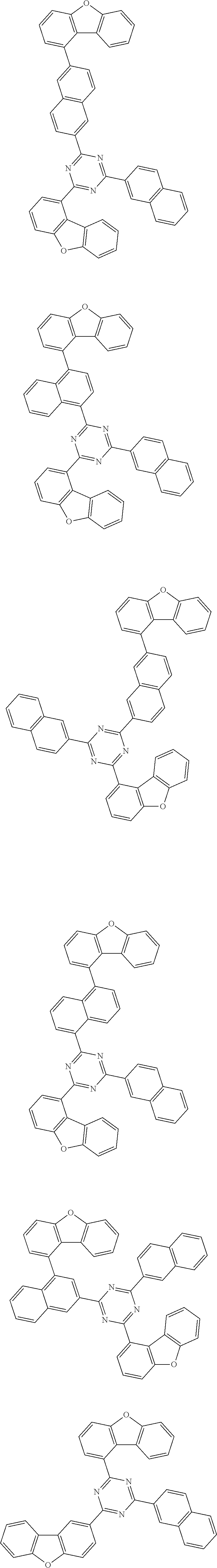

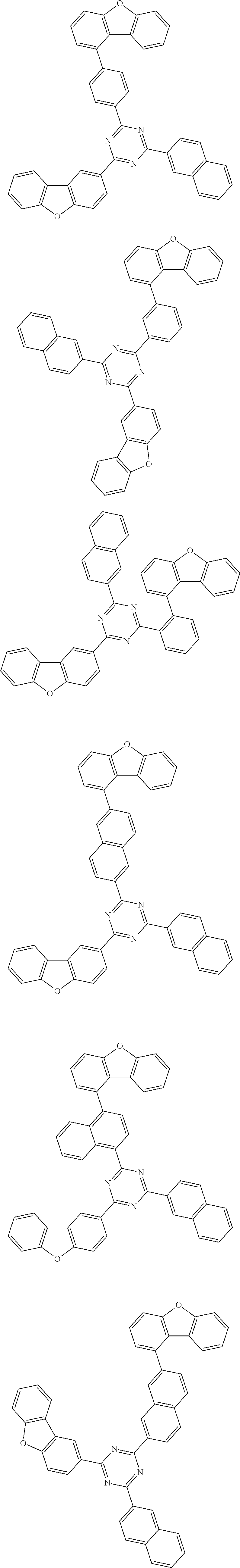

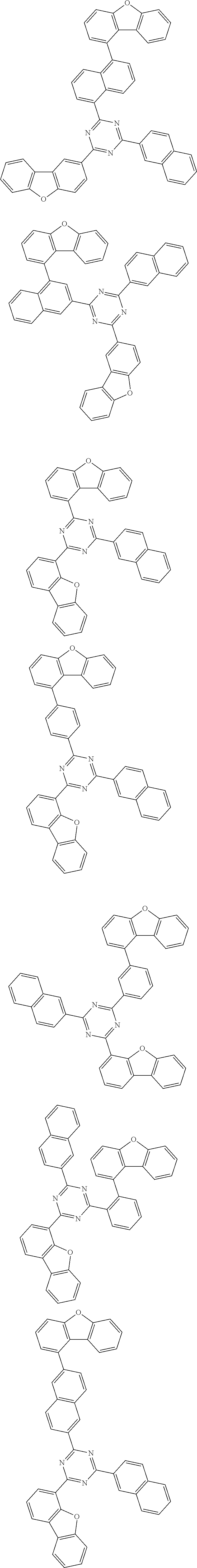

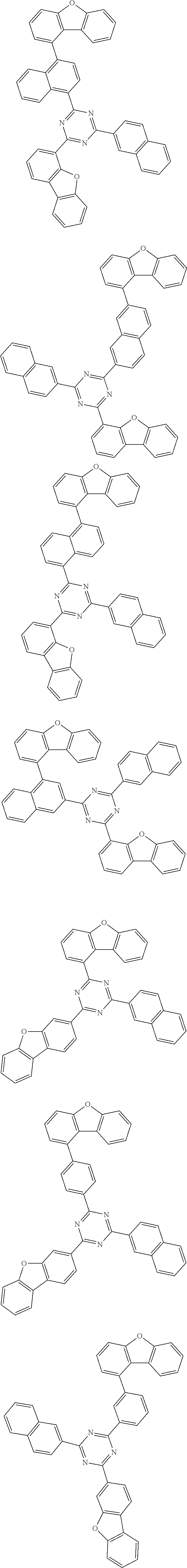

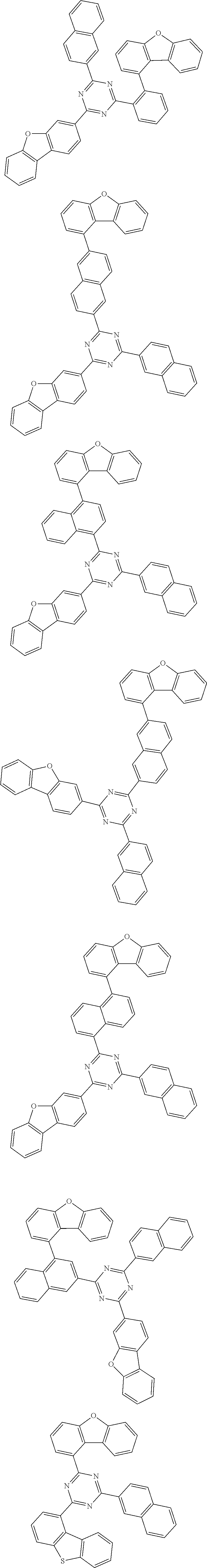

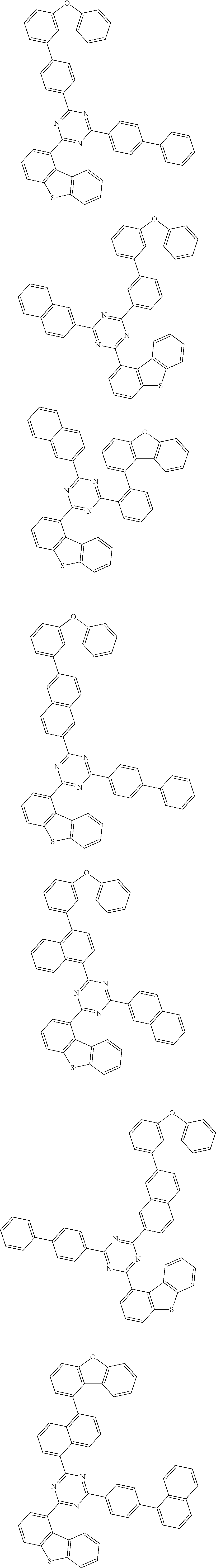

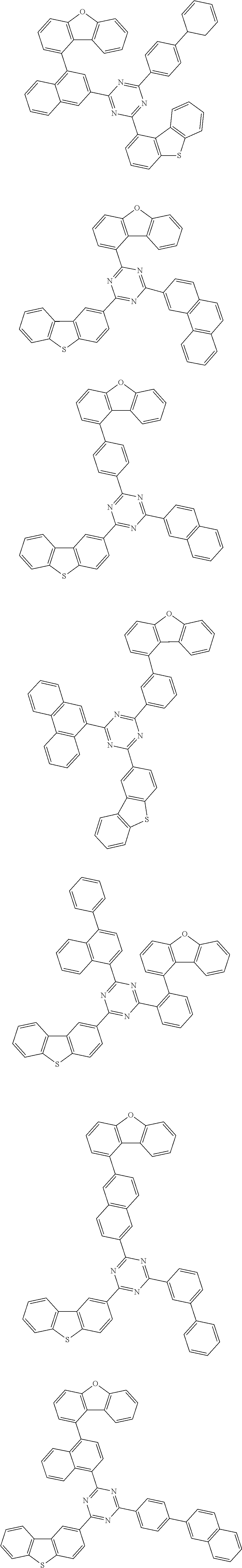

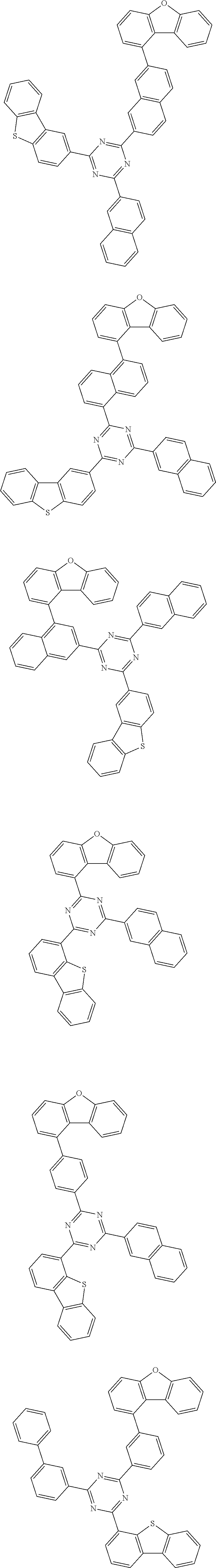

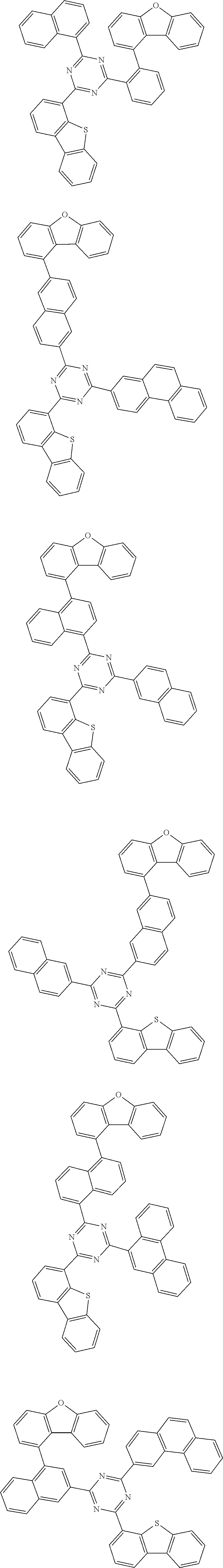

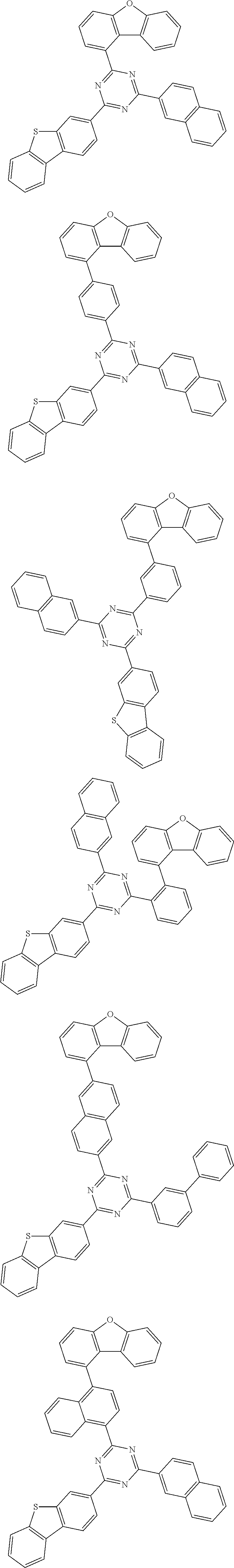

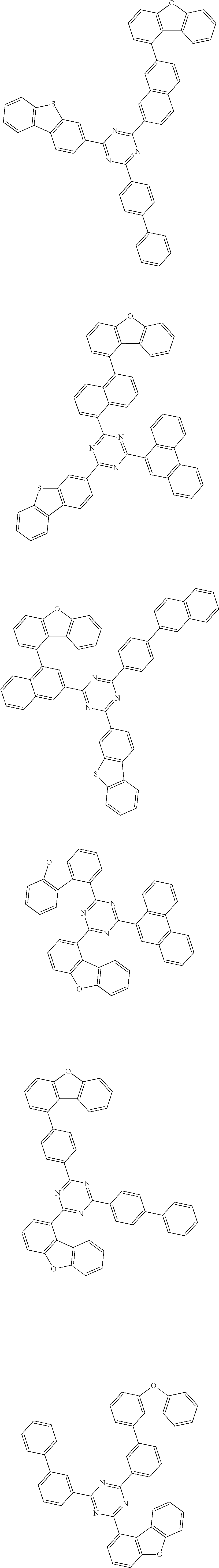

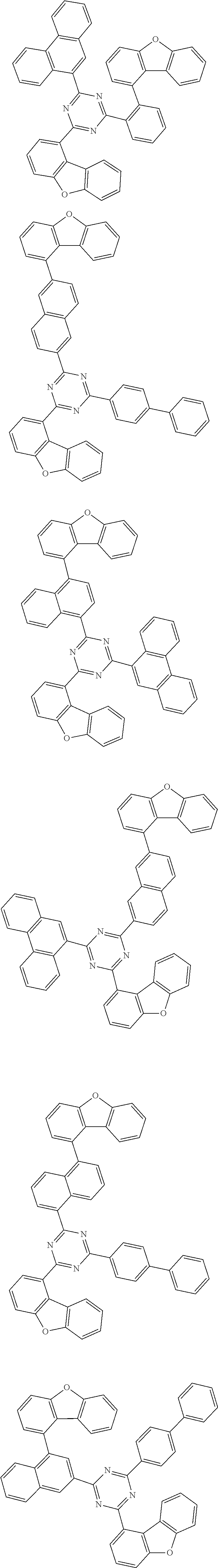

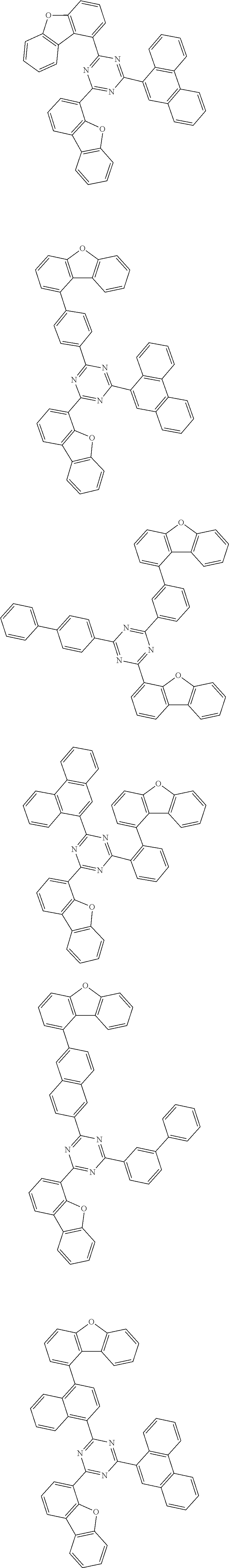

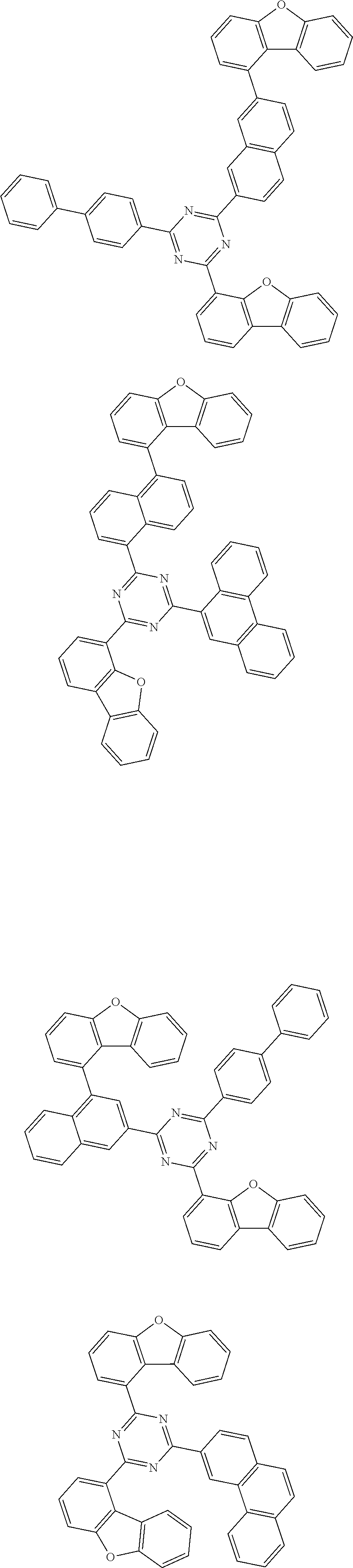 [SMILES: c1ccc(-c2ccc(-c3nc(-c4ccc5ccc(-c6cccc7oc8ccccc8c67)cc5c4)nc(-c4cccc5c4oc4ccccc45)n3)cc2)cc1 ; c1ccc2c(c1)cc(-c1nc(-c3cccc4c(-c5cccc6oc7ccccc7c56)cccc34)nc(-c3cccc4c3oc3ccccc34)n1)c1ccccc12 ; c1ccc(-c2ccc(-c3nc(-c4cc(-c5cccc6oc7ccccc7c56)c5ccccc5c4)nc(-c4cccc5c4oc4ccccc45)n3)cc2)cc1 ; c1ccc2c(c1)ccc1ccc(-c3nc(-c4cccc5oc6ccccc6c45)nc(-c4cccc5oc6ccccc6c45)n3)cc12]

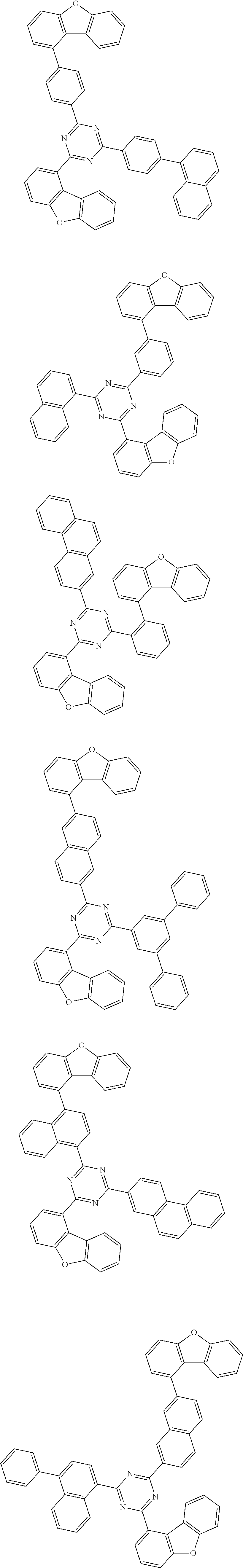

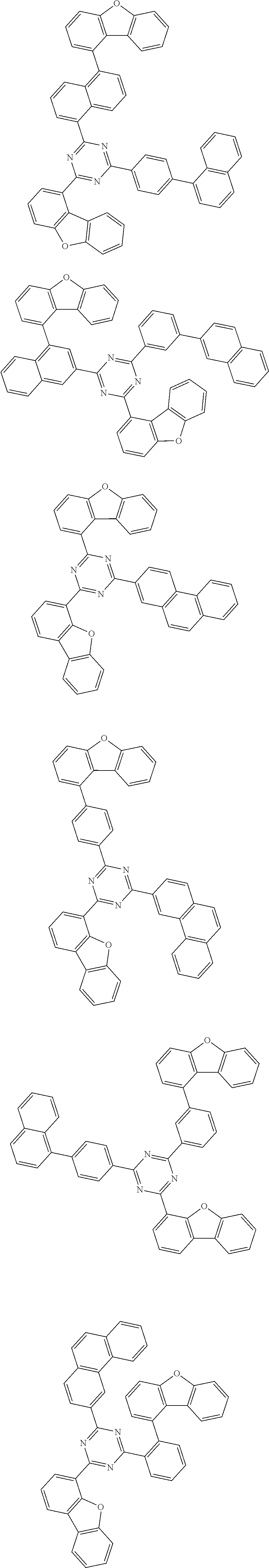

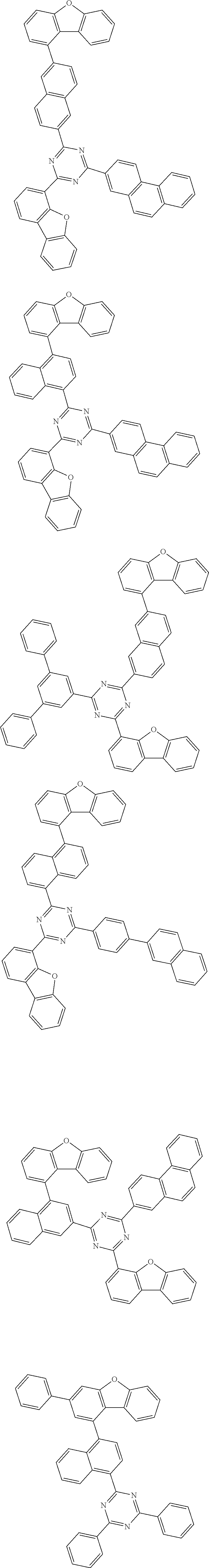

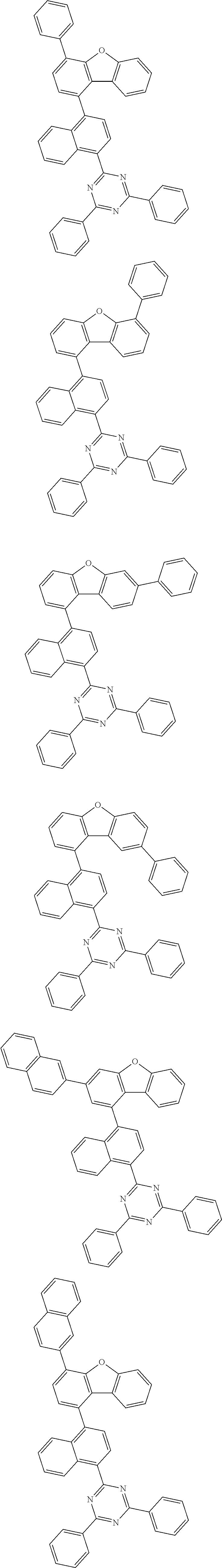

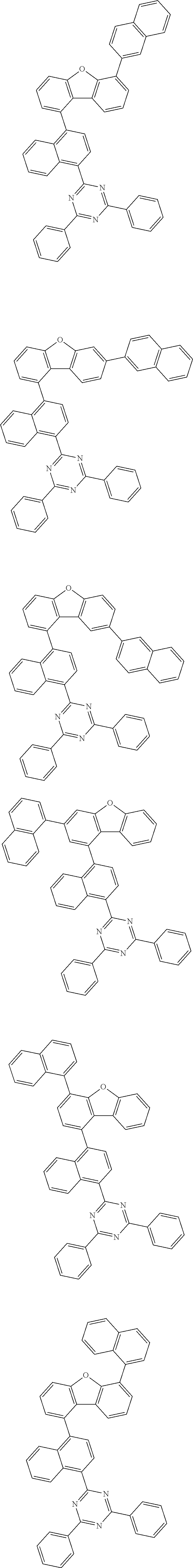

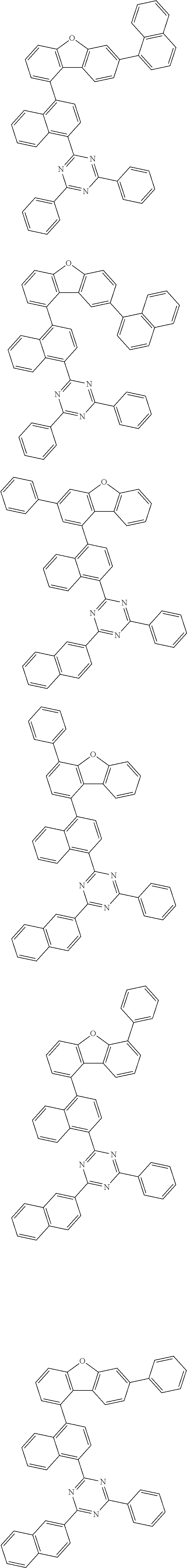

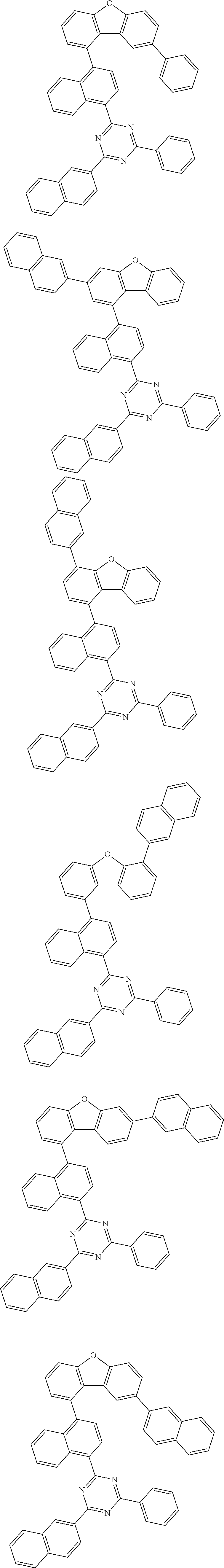

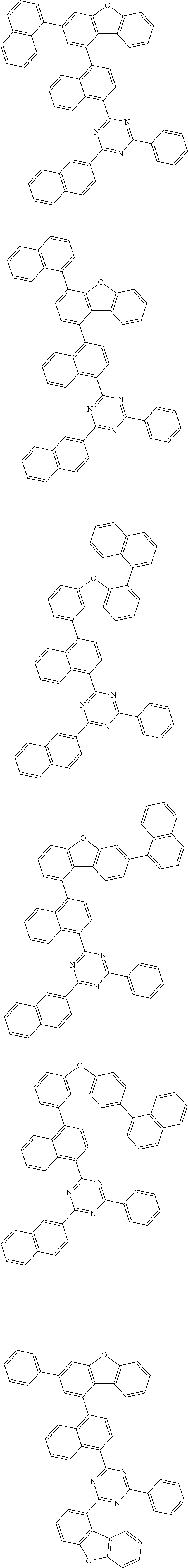

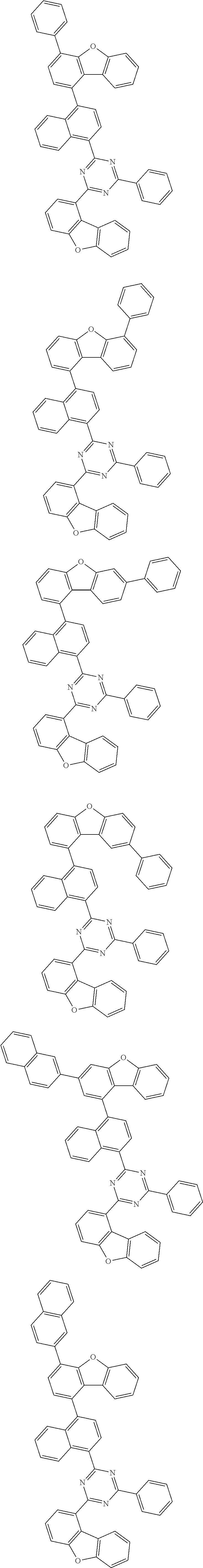

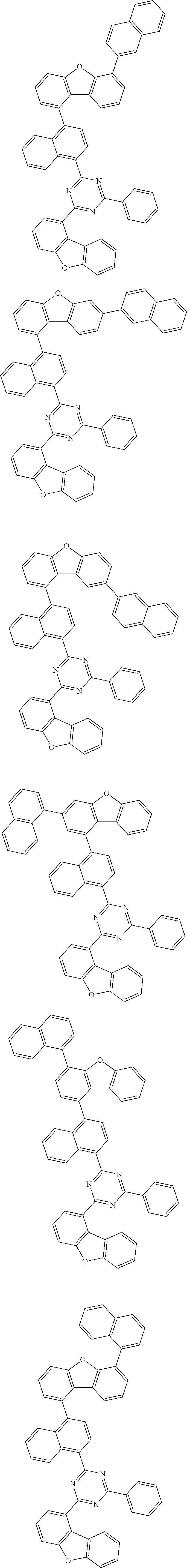

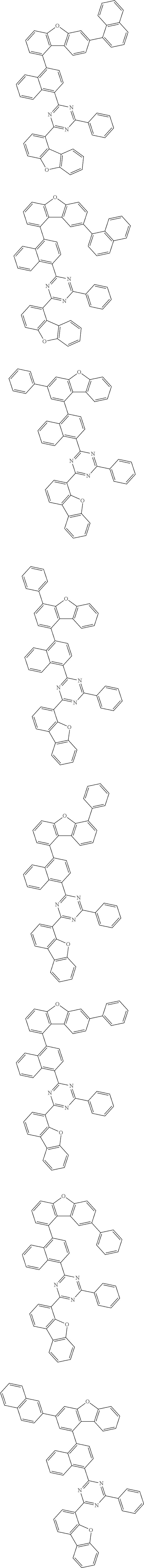

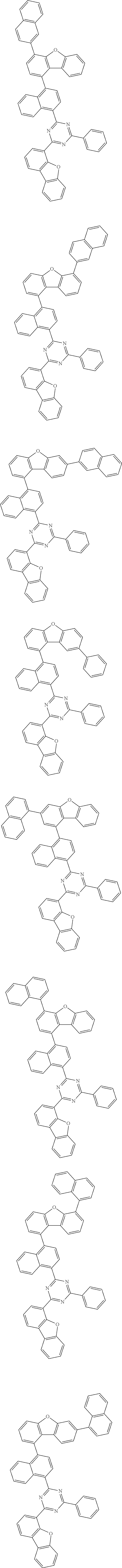

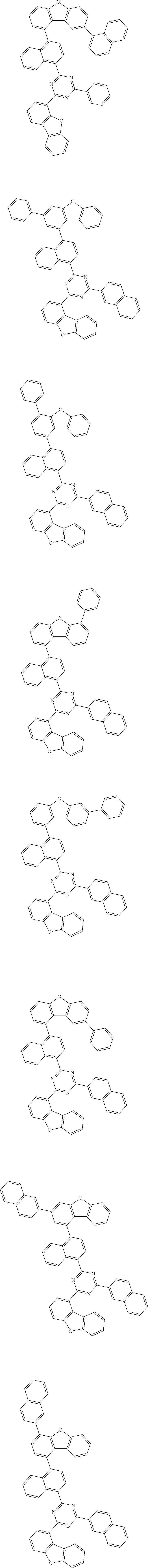

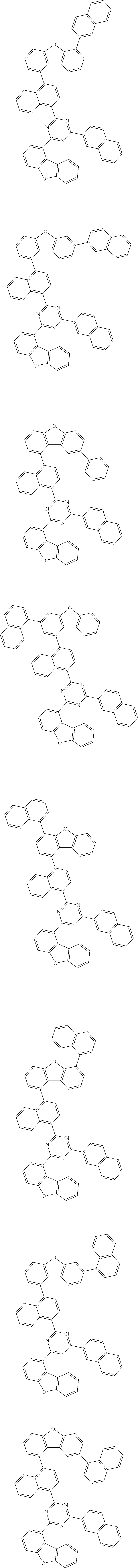

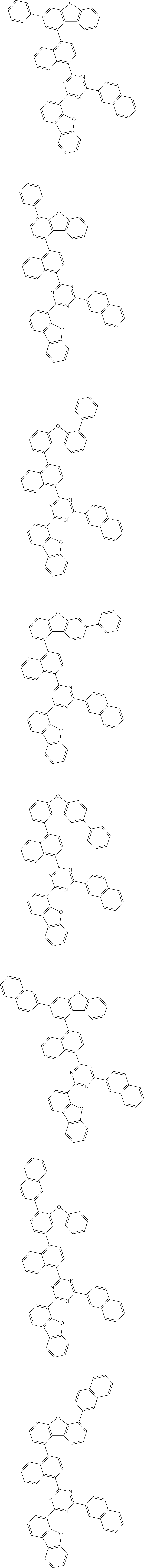

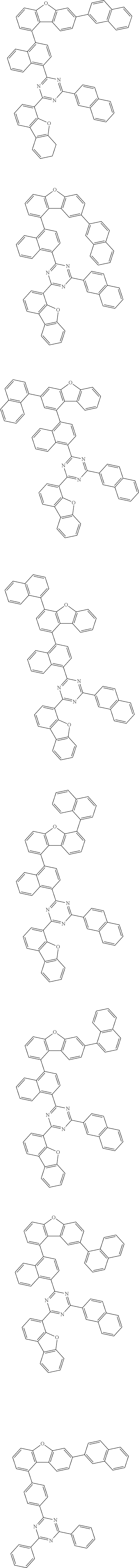

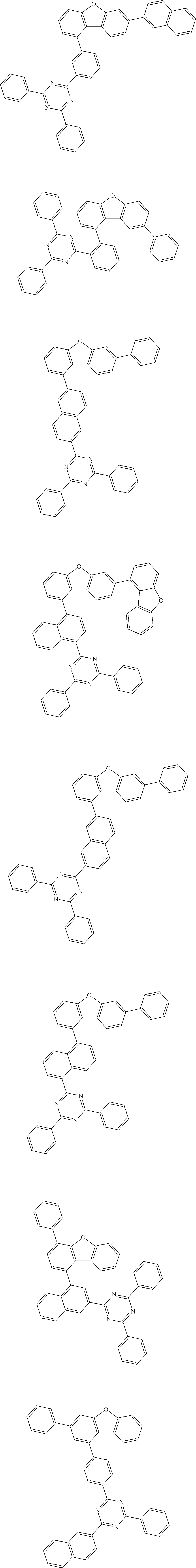

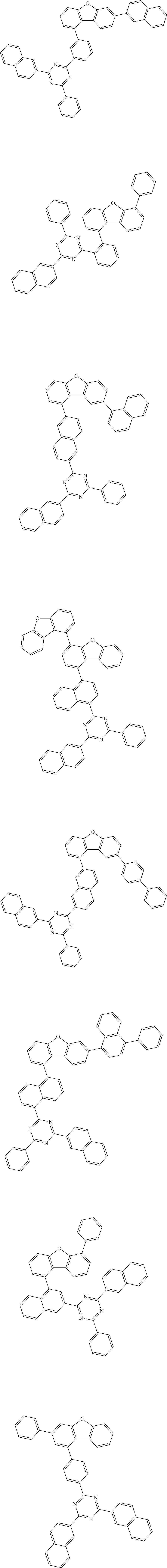

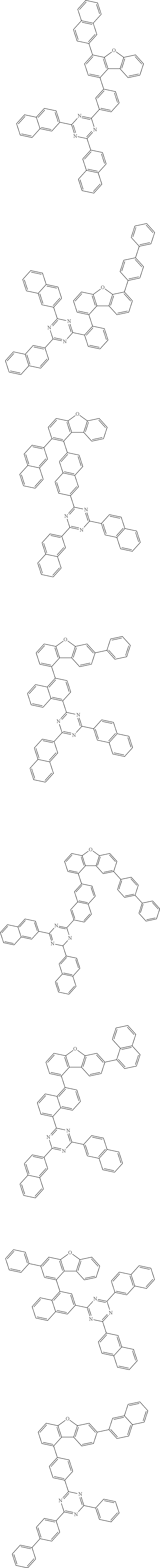

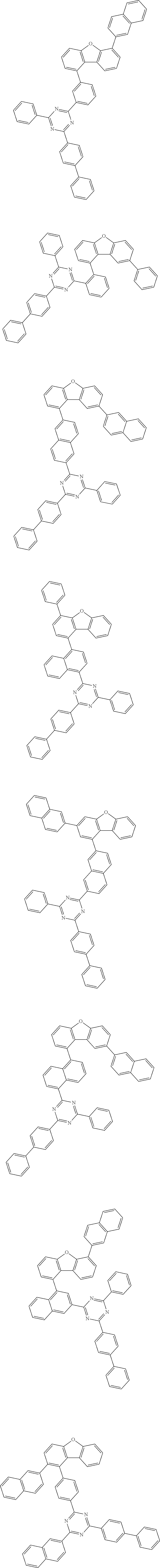

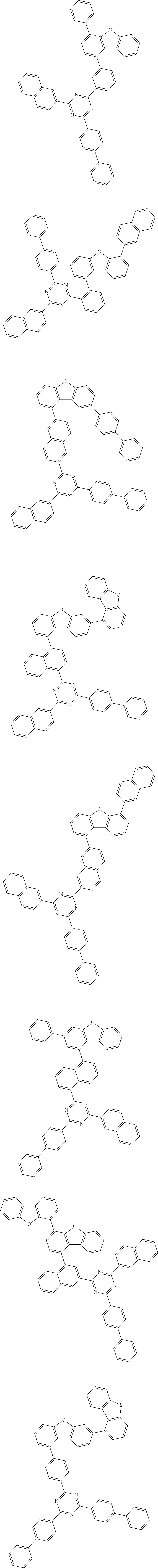

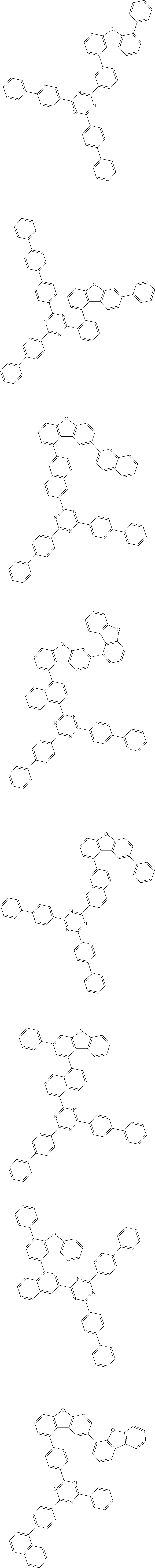

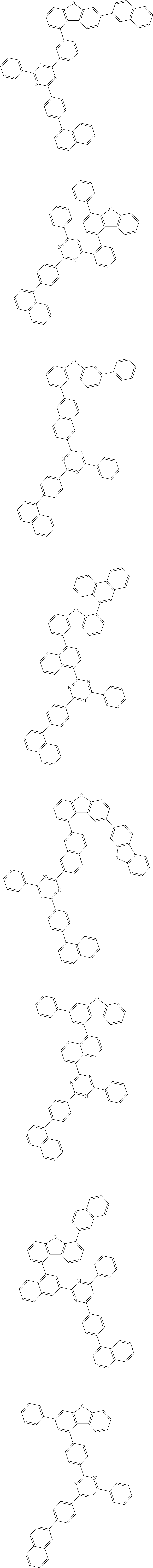

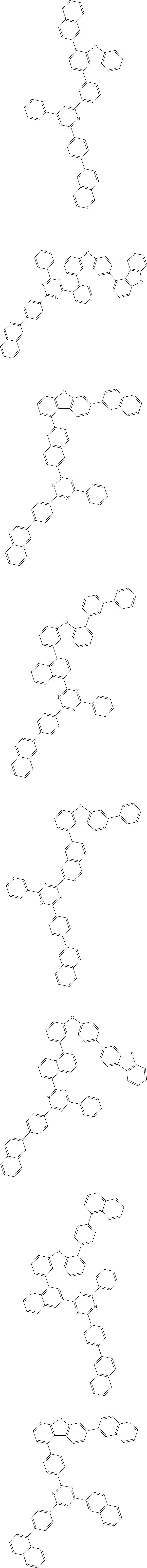

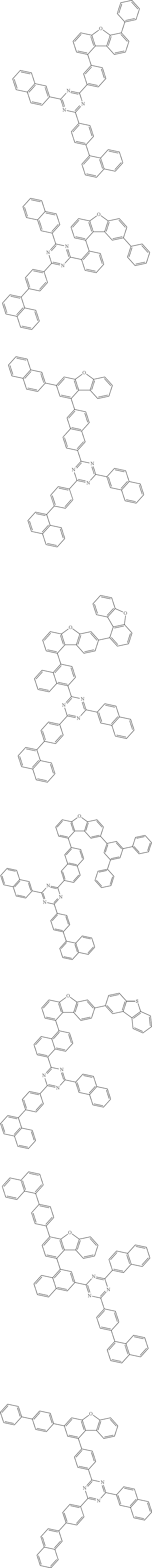

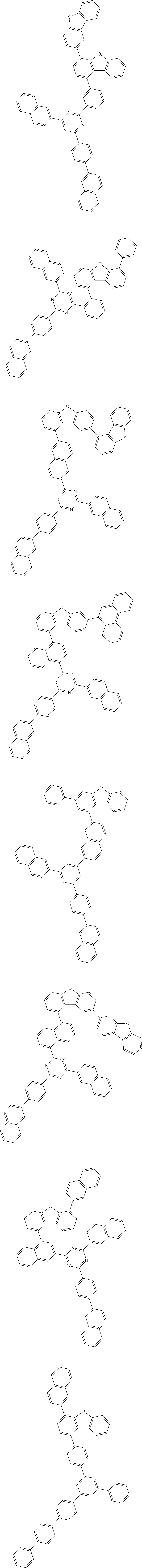

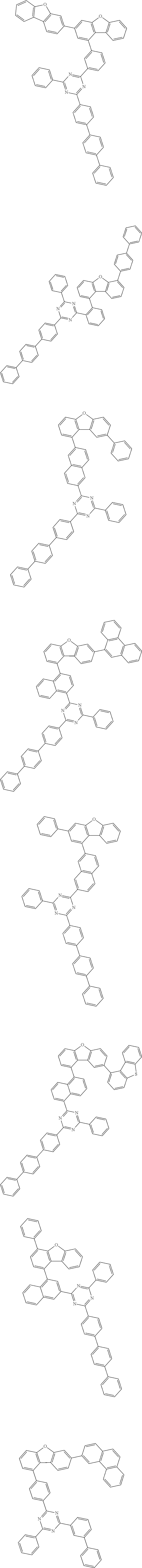

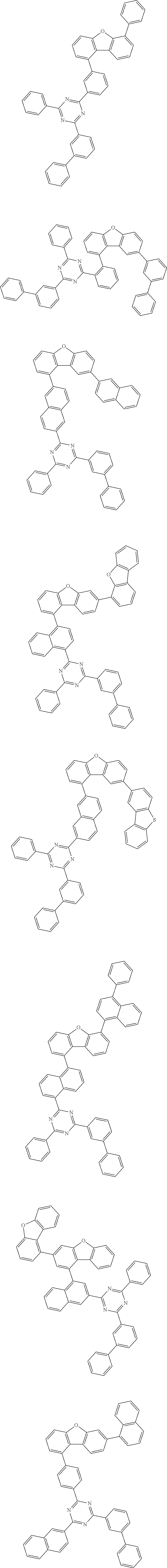

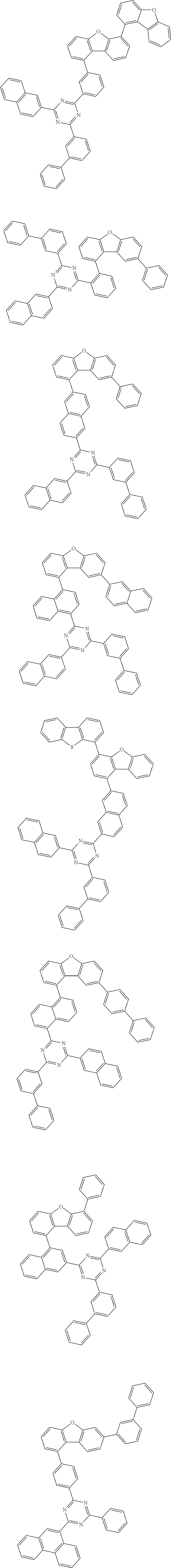

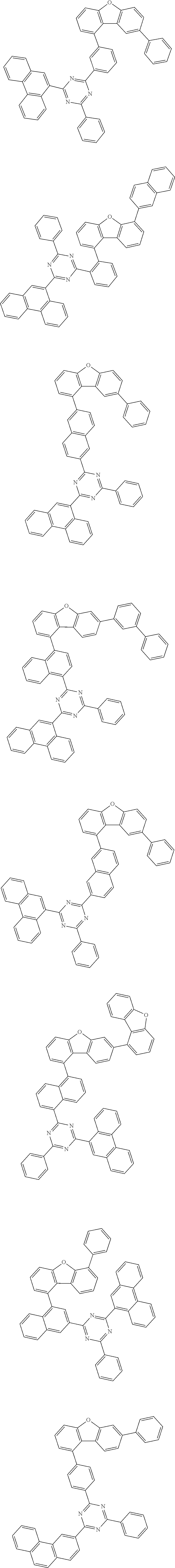

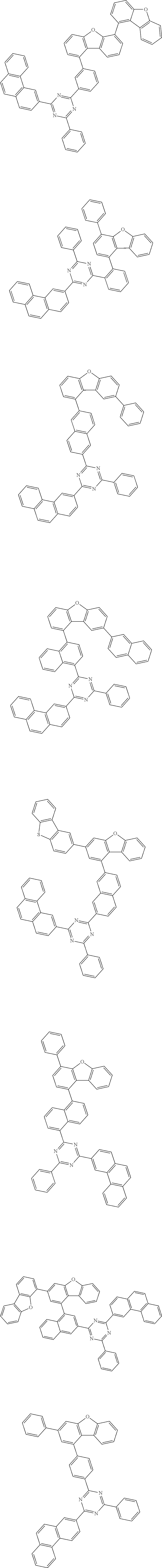

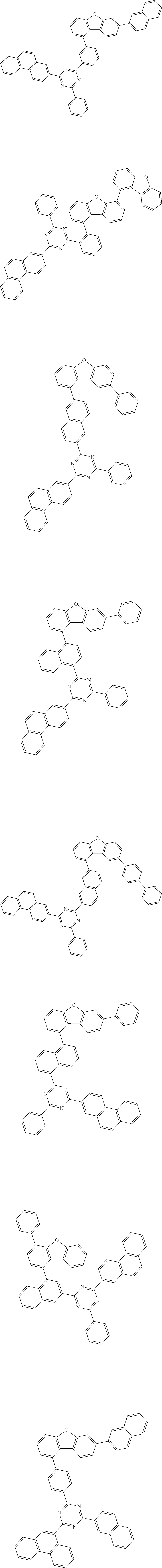

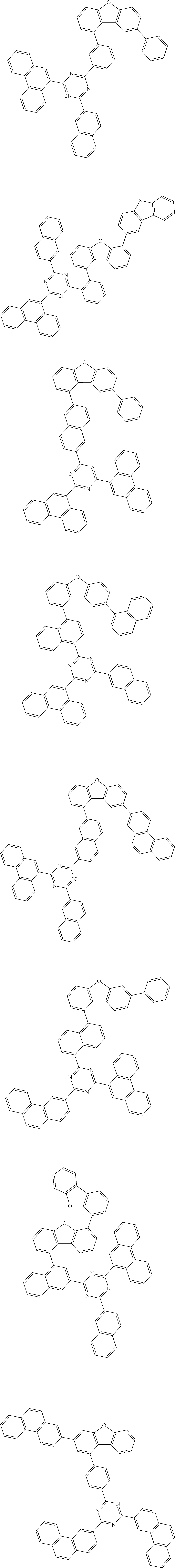

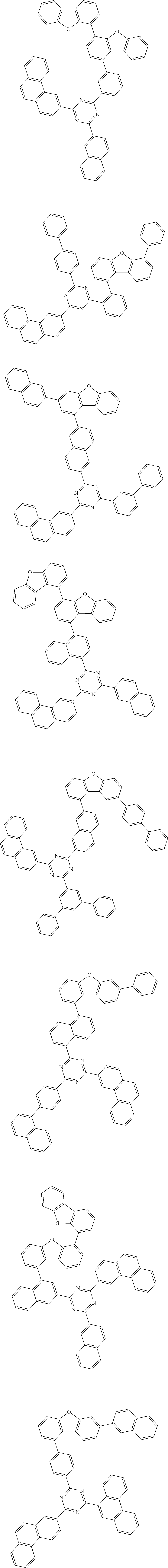

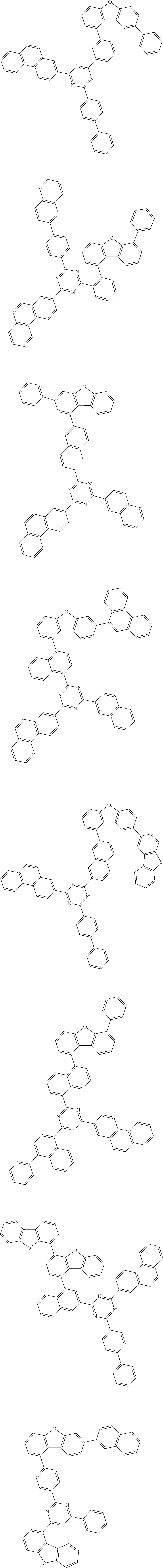

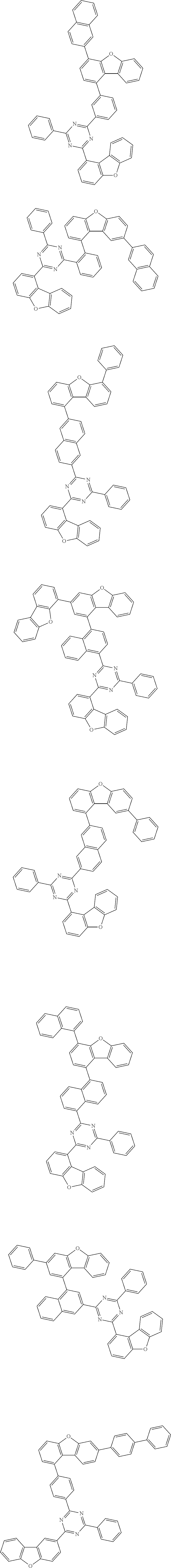

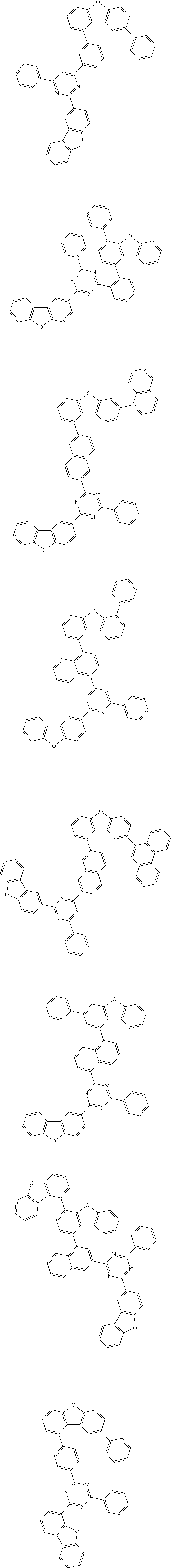

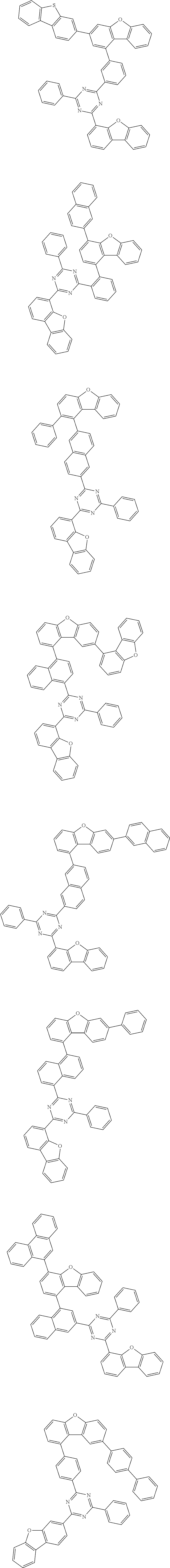

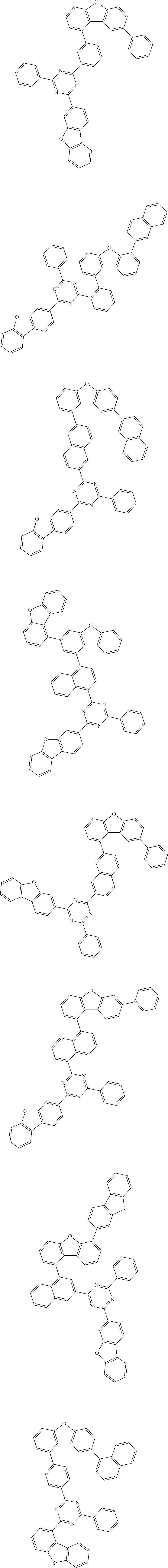

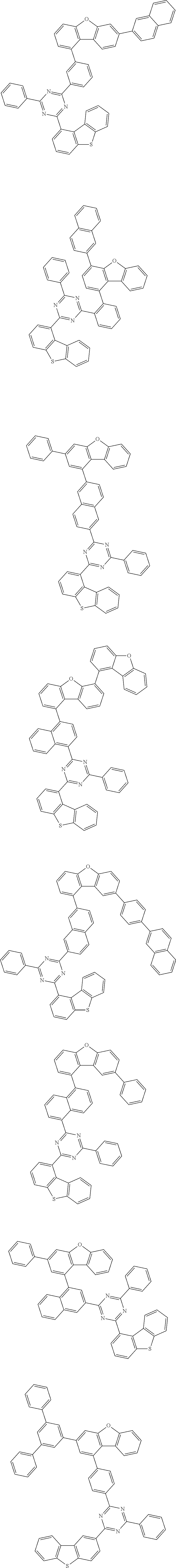

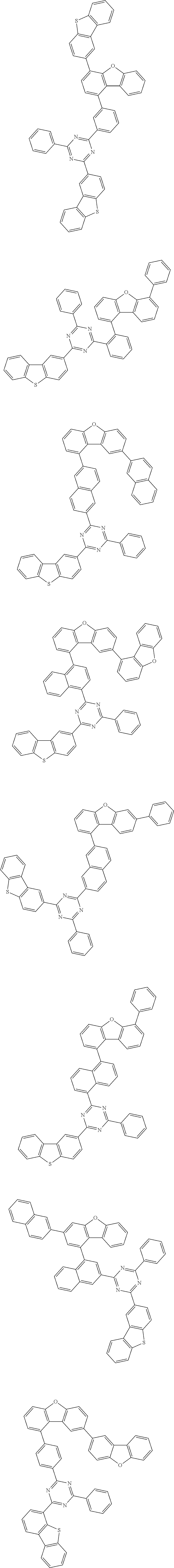

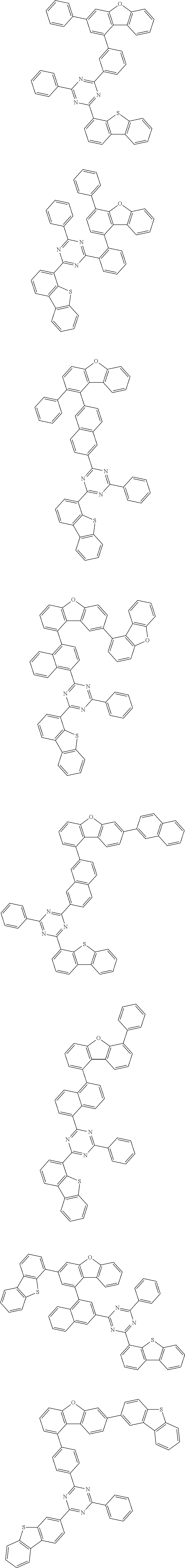

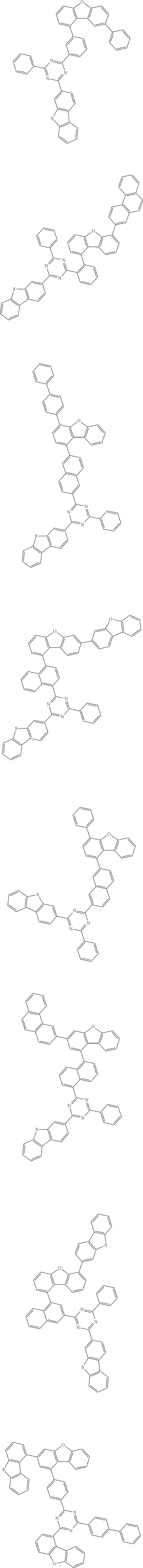

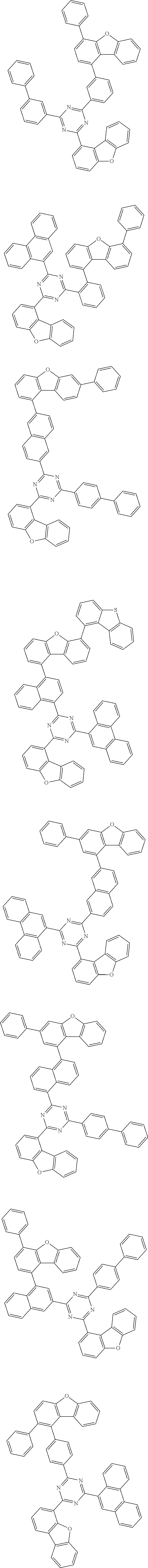

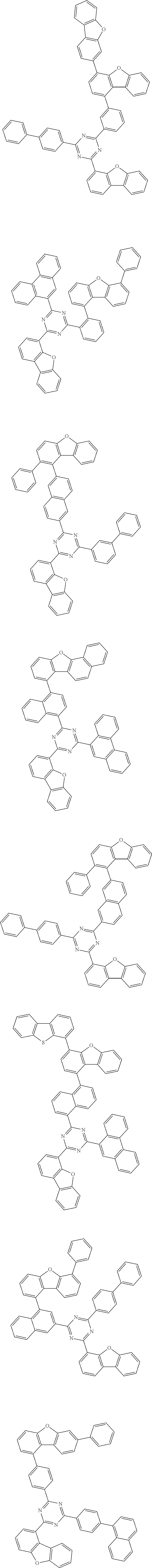

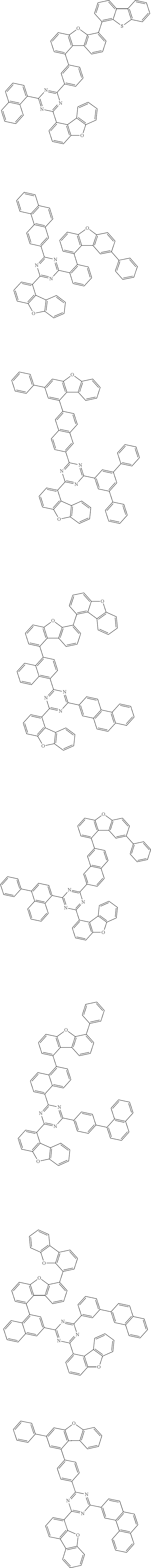

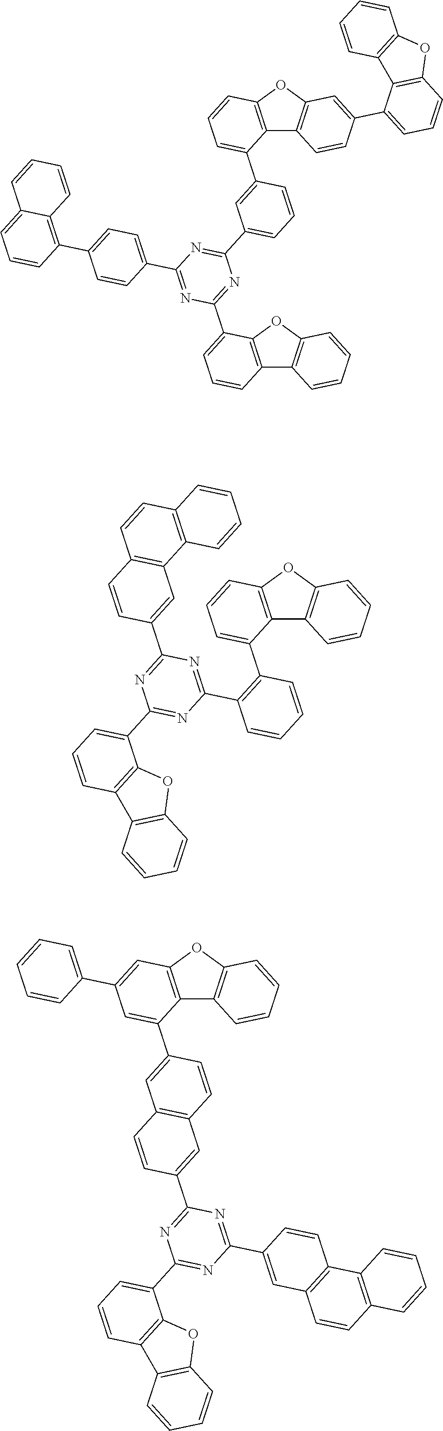 [SMILES: c1cc(-c2nc(-c3ccc(-c4cccc5ccccc45)cc3)nc(-c3cccc4c3oc3ccccc34)n2)cc(-c2cccc3oc4cc(-c5cccc6oc7ccccc7c56)ccc4c23)c1 ; c1ccc(-c2cccc3oc4ccccc4c23)c(-c2nc(-c3ccc4ccc5ccccc5c4c3)nc(-c3cccc4c3oc3ccccc34)n2)c1 ; c1ccc(-c2cc(-c3ccc4cc(-c5nc(-c6ccc7c(ccc8ccccc87)c6)nc(-c6cccc7c6oc6ccccc67)n5)ccc4c3)c3c(c2)oc2ccccc23)cc1]

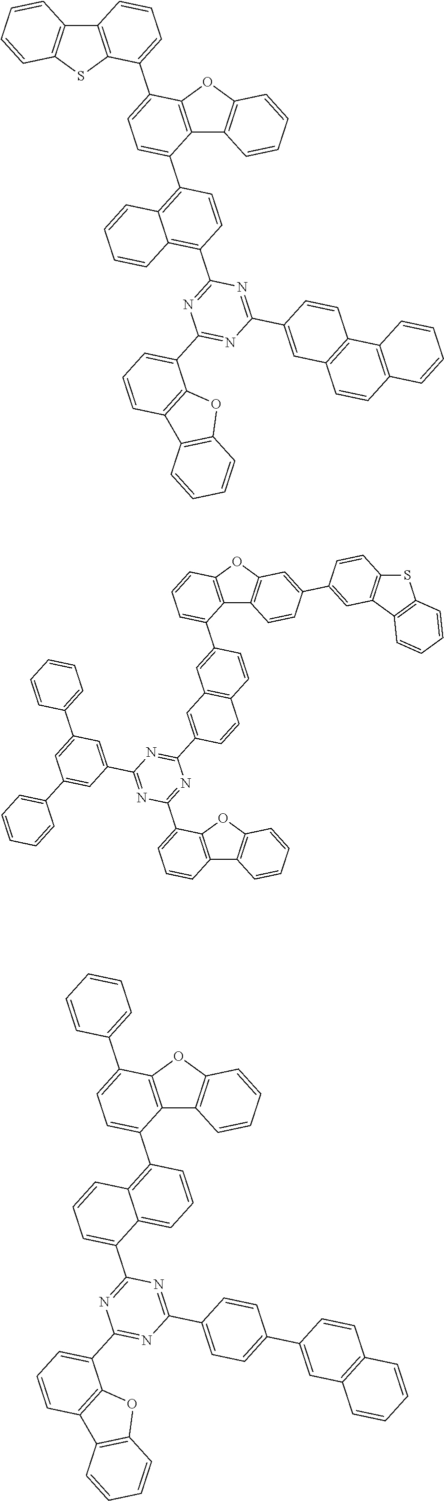 [SMILES: c1ccc2c(c1)ccc1cc(-c3nc(-c4ccc(-c5ccc(-c6cccc7c6sc6ccccc67)c6oc7ccccc7c56)c5ccccc45)nc(-c4cccc5c4oc4ccccc45)n3)ccc12 ; c1ccc(-c2cc(-c3ccccc3)cc(-c3nc(-c4ccc5ccc(-c6cccc7oc8cc(-c9ccc%10sc%11ccccc%11c%10c9)ccc8c67)cc5c4)nc(-c4cccc5c4oc4ccccc45)n3)c2)cc1 ; c1ccc(-c2ccc(-c3cccc4c(-c5nc(-c6ccc(-c7ccc8ccccc8c7)cc6)nc(-c6cccc7c6oc6ccccc67)n5)cccc34)c3c2oc2ccccc23)cc1]

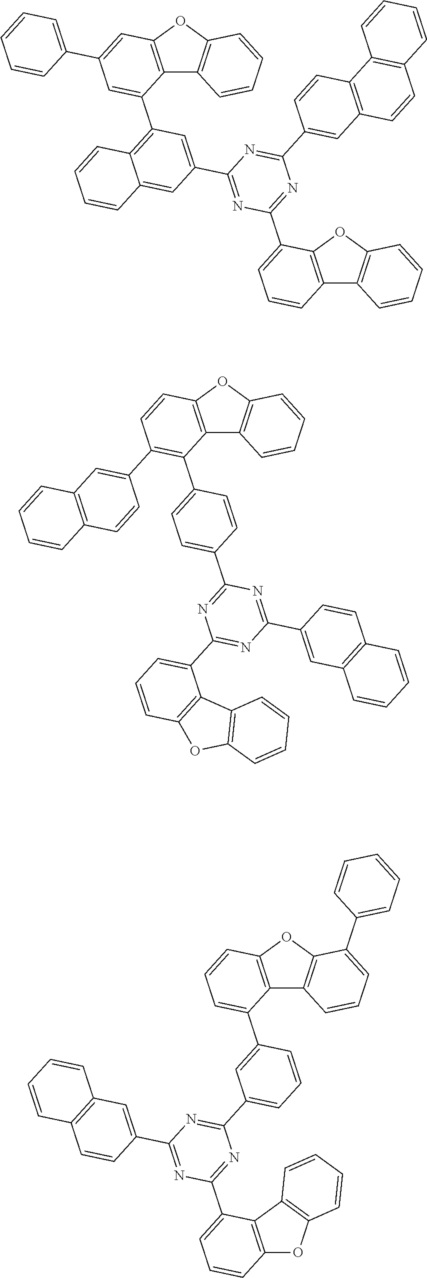 [SMILES: c1ccc(-c2cc(-c3cc(-c4nc(-c5ccc6c(ccc7ccccc76)c5)nc(-c5cccc6c5oc5ccccc56)n4)cc4ccccc34)c3c(c2)oc2ccccc23)cc1 ; c1ccc2cc(-c3nc(-c4ccc(-c5c(-c6ccc7ccccc7c6)ccc6oc7ccccc7c56)cc4)nc(-c4cccc5oc6ccccc6c45)n3)ccc2c1 ; c1ccc(-c2cccc3c2oc2cccc(-c4cccc(-c5nc(-c6ccc7ccccc7c6)nc(-c6cccc7oc8ccccc8c67)n5)c4)c23)cc1]

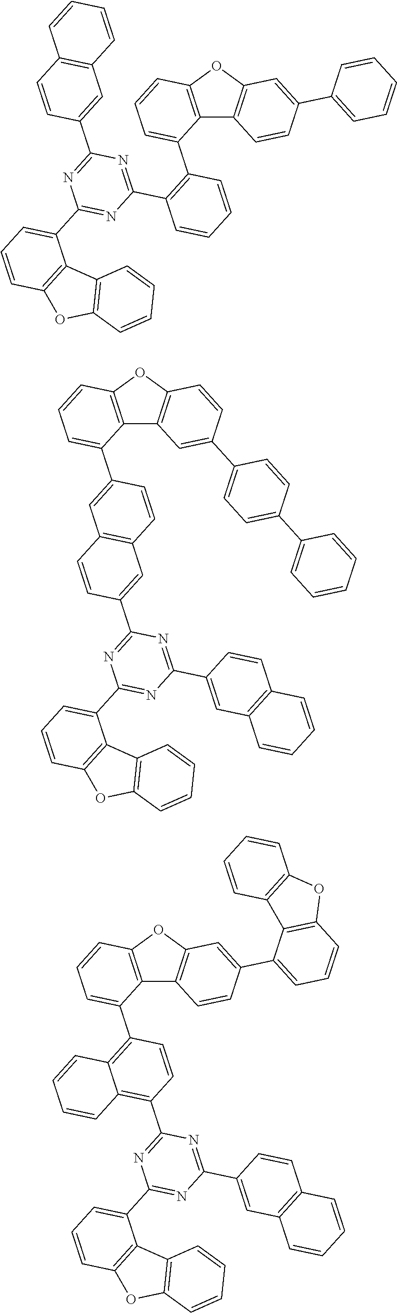

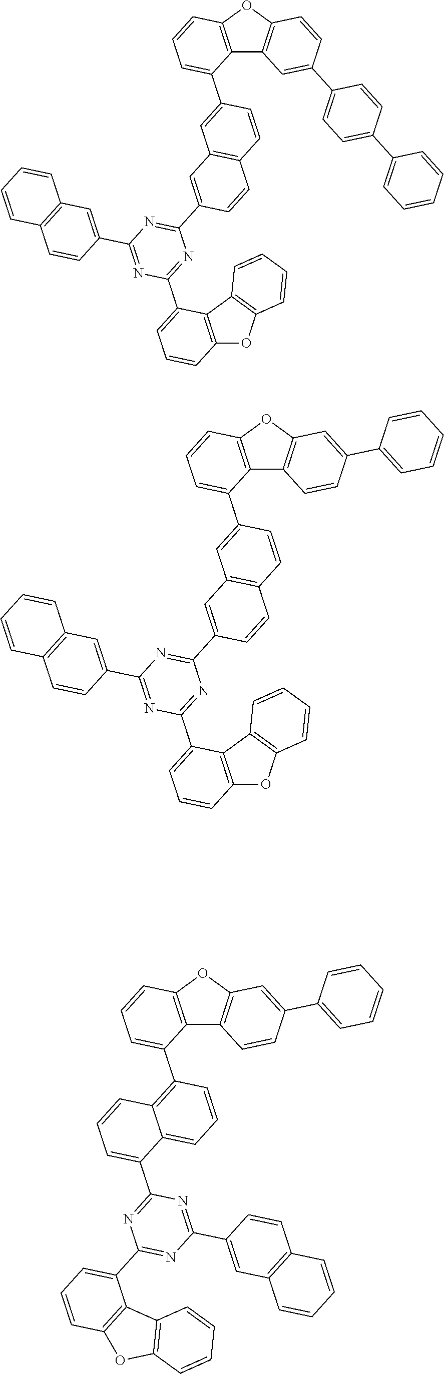 [SMILES: c1ccc(-c2ccc(-c3ccc4oc5cccc(-c6ccc7ccc(-c8nc(-c9ccc%10ccccc%10c9)nc(-c9cccc%10oc%11ccccc%11c9%10)n8)cc7c6)c5c4c3)cc2)cc1 ; c1ccc(-c2ccc3c(c2)oc2cccc(-c4ccc5ccc(-c6nc(-c7ccc8ccccc8c7)nc(-c7cccc8oc9ccccc9c78)n6)cc5c4)c23)cc1 ; c1ccc(-c2ccc3c(c2)oc2cccc(-c4cccc5c(-c6nc(-c7ccc8ccccc8c7)nc(-c7cccc8oc9ccccc9c78)n6)cccc45)c23)cc1]

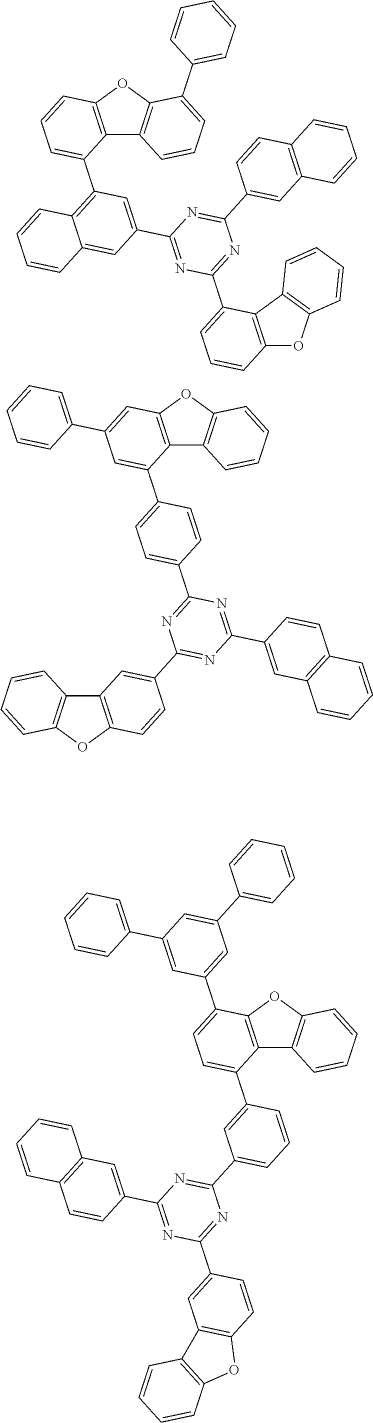

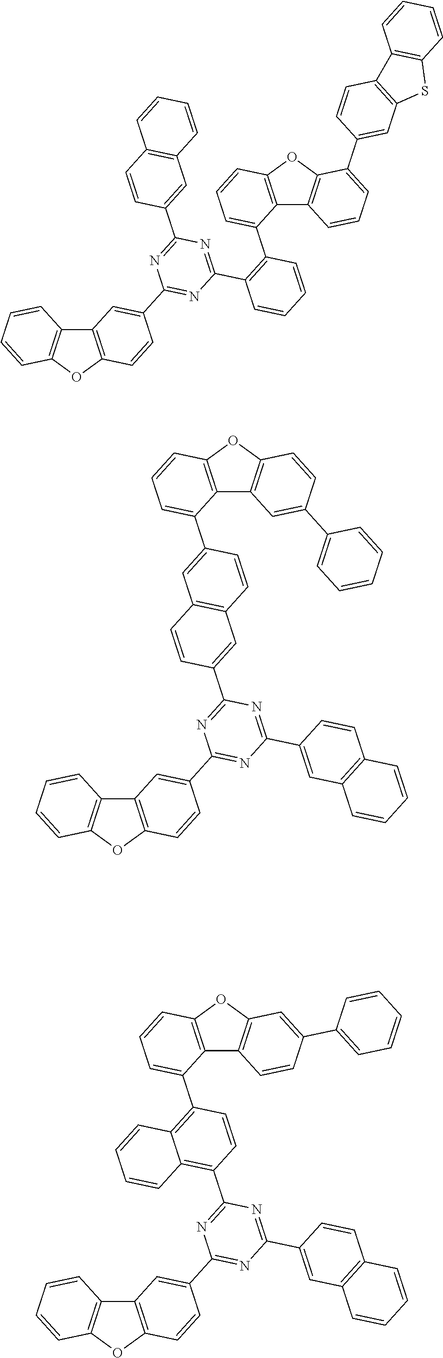 [SMILES: c1ccc(-c2cccc3oc4c(-c5ccc6c(c5)sc5ccccc56)cccc4c23)c(-c2nc(-c3ccc4ccccc4c3)nc(-c3ccc4oc5ccccc5c4c3)n2)c1 ; c1ccc(-c2ccc3oc4cccc(-c5ccc6cc(-c7nc(-c8ccc9ccccc9c8)nc(-c8ccc9oc%10ccccc%10c9c8)n7)ccc6c5)c4c3c2)cc1 ; c1ccc(-c2ccc3c(c2)oc2cccc(-c4ccc(-c5nc(-c6ccc7ccccc7c6)nc(-c6ccc7oc8ccccc8c7c6)n5)c5ccccc45)c23)cc1]

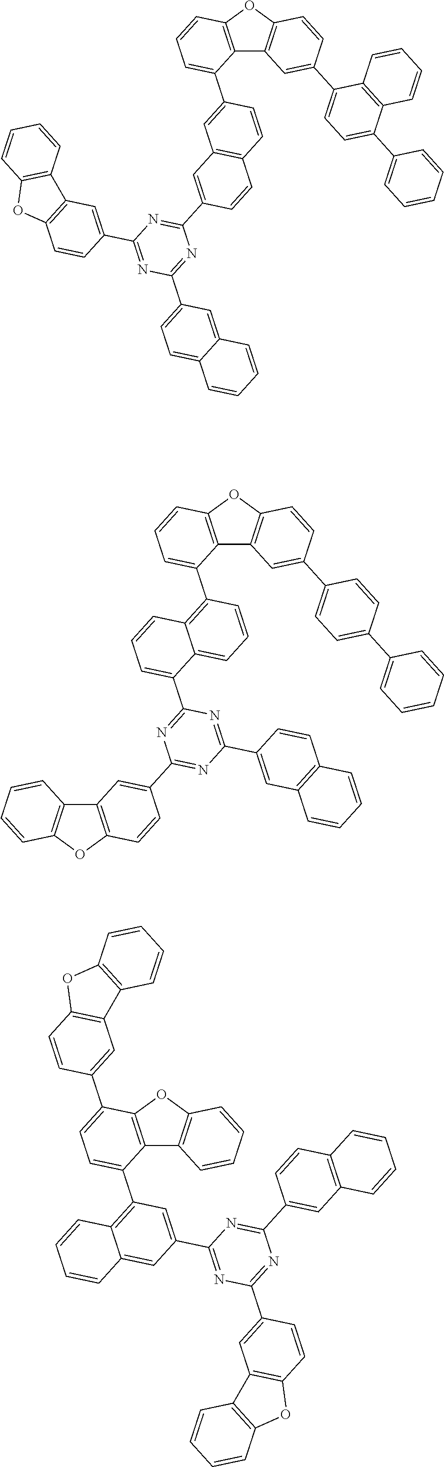 [SMILES: c1ccc(-c2ccc(-c3ccc4oc5cccc(-c6ccc7ccc(-c8nc(-c9ccc%10ccccc%10c9)nc(-c9ccc%10oc%11ccccc%11c%10c9)n8)cc7c6)c5c4c3)c3ccccc23)cc1 ; c1ccc(-c2ccc(-c3ccc4oc5cccc(-c6cccc7c(-c8nc(-c9ccc%10ccccc%10c9)nc(-c9ccc%10oc%11ccccc%11c%10c9)n8)cccc67)c5c4c3)cc2)cc1 ; c1ccc2cc(-c3nc(-c4cc(-c5ccc(-c6ccc7oc8ccccc8c7c6)c6oc7ccccc7c56)c5ccccc5c4)nc(-c4ccc5oc6ccccc6c5c4)n3)ccc2c1]

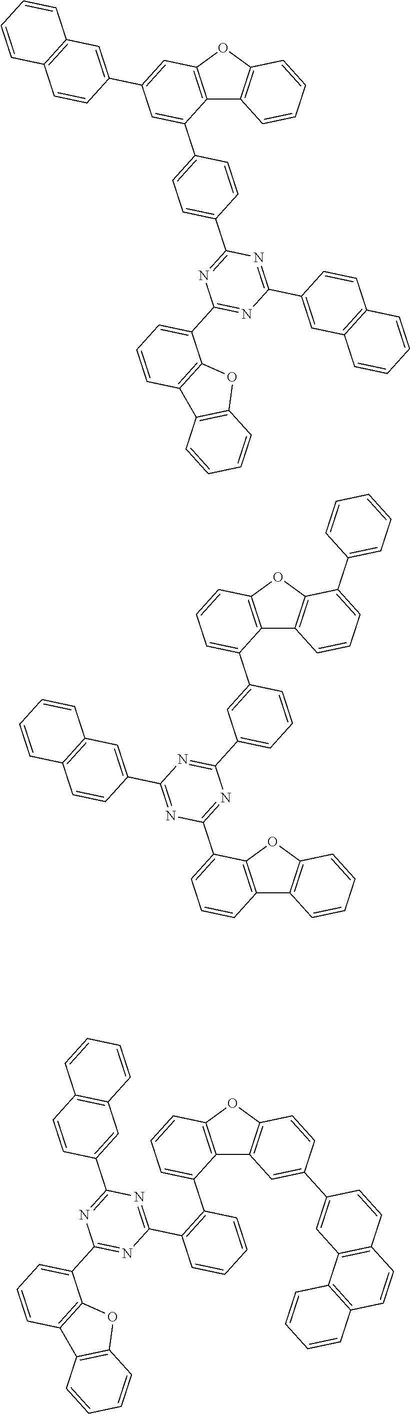 [SMILES: c1ccc2cc(-c3cc(-c4ccc(-c5nc(-c6ccc7ccccc7c6)nc(-c6cccc7c6oc6ccccc67)n5)cc4)c4c(c3)oc3ccccc34)ccc2c1 ; c1ccc(-c2cccc3c2oc2cccc(-c4cccc(-c5nc(-c6ccc7ccccc7c6)nc(-c6cccc7c6oc6ccccc67)n5)c4)c23)cc1 ; c1ccc(-c2cccc3oc4ccc(-c5ccc6ccc7ccccc7c6c5)cc4c23)c(-c2nc(-c3ccc4ccccc4c3)nc(-c3cccc4c3oc3ccccc34)n2)c1]

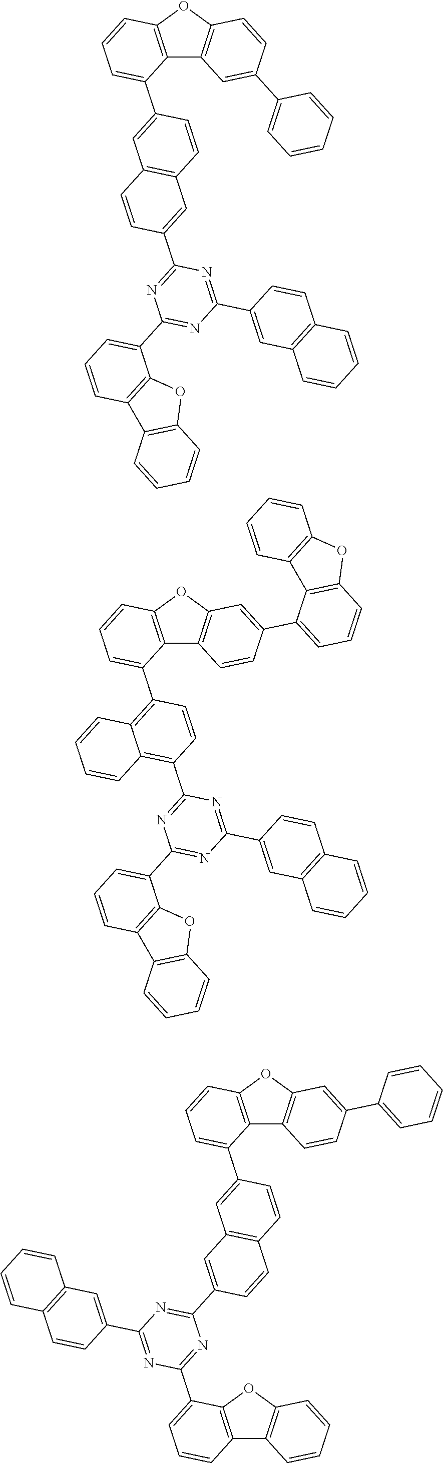 [SMILES: c1ccc(-c2ccc3oc4cccc(-c5ccc6cc(-c7nc(-c8ccc9ccccc9c8)nc(-c8cccc9c8oc8ccccc89)n7)ccc6c5)c4c3c2)cc1 ; c1ccc2cc(-c3nc(-c4ccc(-c5cccc6oc7cc(-c8cccc9oc%10ccccc%10c89)ccc7c56)c5ccccc45)nc(-c4cccc5c4oc4ccccc45)n3)ccc2c1 ; c1ccc(-c2ccc3c(c2)oc2cccc(-c4ccc5ccc(-c6nc(-c7ccc8ccccc8c7)nc(-c7cccc8c7oc7ccccc78)n6)cc5c4)c23)cc1]

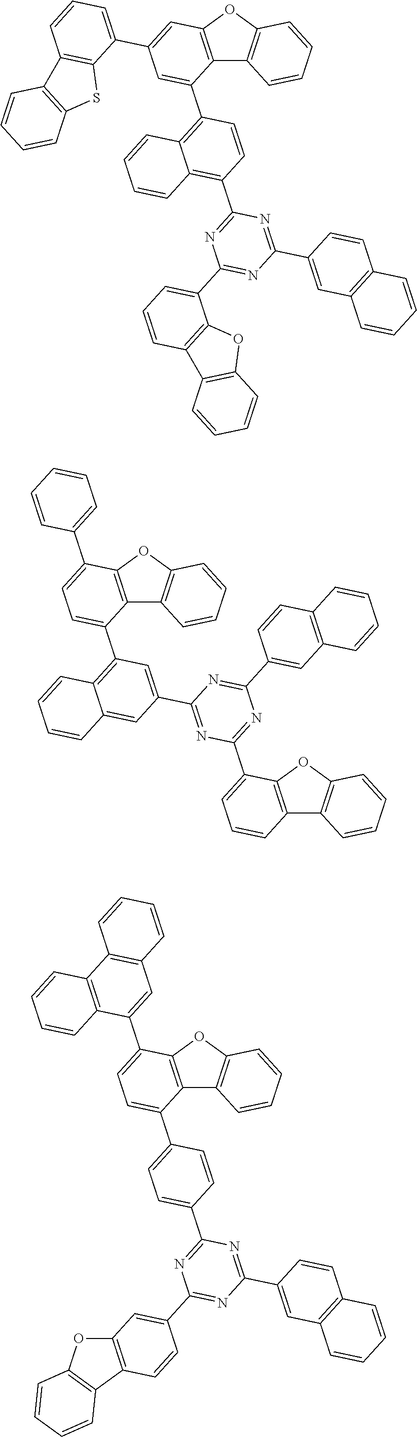 [SMILES: c1ccc2cc(-c3nc(-c4ccc(-c5cc(-c6cccc7c6sc6ccccc67)cc6oc7ccccc7c56)c5ccccc45)nc(-c4cccc5c4oc4ccccc45)n3)ccc2c1 ; c1ccc(-c2ccc(-c3cc(-c4nc(-c5ccc6ccccc6c5)nc(-c5cccc6c5oc5ccccc56)n4)cc4ccccc34)c3c2oc2ccccc23)cc1 ; c1ccc2cc(-c3nc(-c4ccc(-c5ccc(-c6cc7ccccc7c7ccccc67)c6oc7ccccc7c56)cc4)nc(-c4ccc5c(c4)oc4ccccc45)n3)ccc2c1]

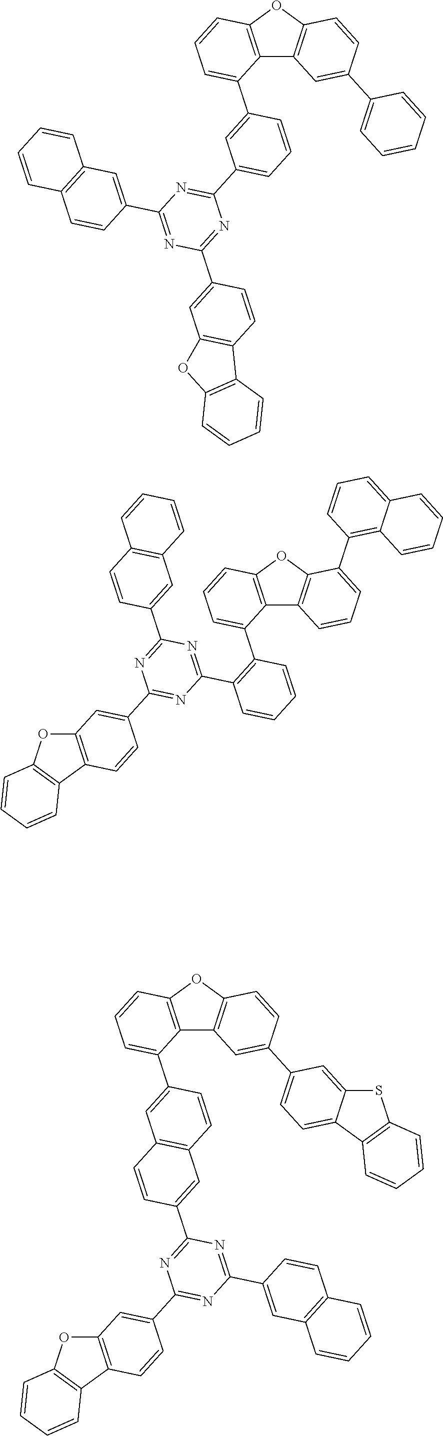 [SMILES: c1ccc(-c2ccc3oc4cccc(-c5cccc(-c6nc(-c7ccc8ccccc8c7)nc(-c7ccc8c(c7)oc7ccccc78)n6)c5)c4c3c2)cc1 ; c1ccc(-c2cccc3oc4c(-c5cccc6ccccc56)cccc4c23)c(-c2nc(-c3ccc4ccccc4c3)nc(-c3ccc4c(c3)oc3ccccc34)n2)c1 ; c1ccc2cc(-c3nc(-c4ccc5cc(-c6cccc7oc8ccc(-c9ccc%10c(c9)sc9ccccc9%10)cc8c67)ccc5c4)nc(-c4ccc5c(c4)oc4ccccc45)n3)ccc2c1]

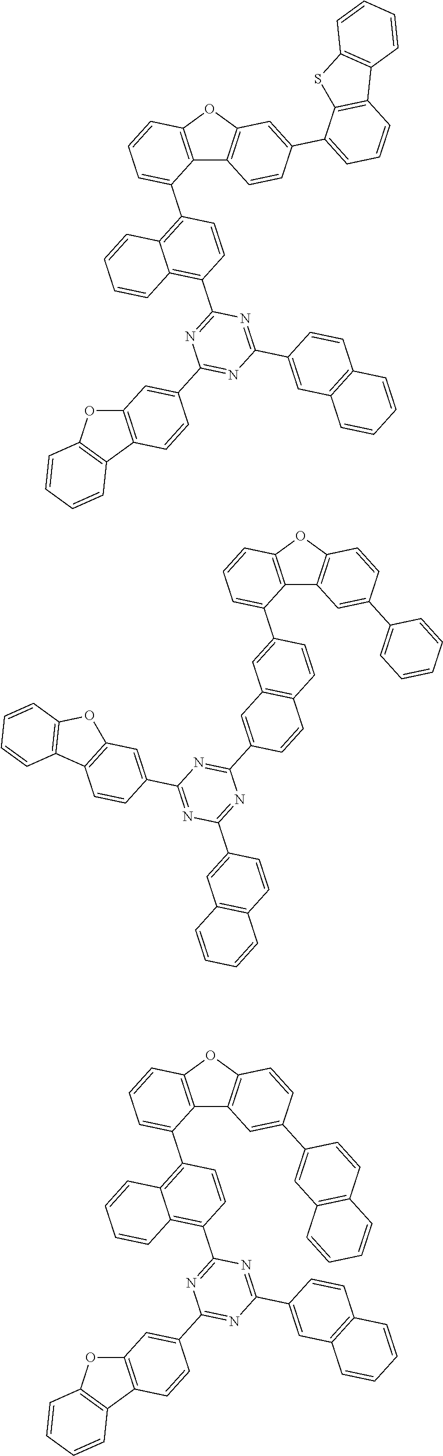 [SMILES: c1ccc2cc(-c3nc(-c4ccc5c(c4)oc4ccccc45)nc(-c4ccc(-c5cccc6oc7cc(-c8cccc9c8sc8ccccc89)ccc7c56)c5ccccc45)n3)ccc2c1 ; c1ccc(-c2ccc3oc4cccc(-c5ccc6ccc(-c7nc(-c8ccc9ccccc9c8)nc(-c8ccc9c(c8)oc8ccccc89)n7)cc6c5)c4c3c2)cc1 ; c1ccc2cc(-c3ccc4oc5cccc(-c6ccc(-c7nc(-c8ccc9ccccc9c8)nc(-c8ccc9c(c8)oc8ccccc89)n7)c7ccccc67)c5c4c3)ccc2c1]

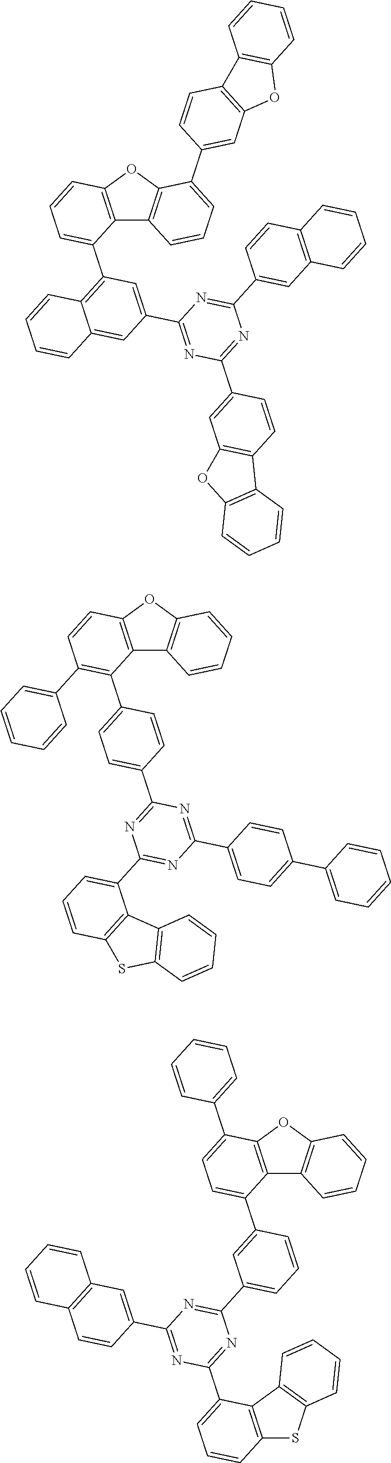 [SMILES: c1ccc2cc(-c3nc(-c4cc(-c5cccc6oc7c(-c8ccc9c(c8)oc8ccccc89)cccc7c56)c5ccccc5c4)nc(-c4ccc5c(c4)oc4ccccc45)n3)ccc2c1 ; c1ccc(-c2ccc(-c3nc(-c4ccc(-c5c(-c6ccccc6)ccc6oc7ccccc7c56)cc4)nc(-c4cccc5sc6ccccc6c45)n3)cc2)cc1 ; c1ccc(-c2ccc(-c3cccc(-c4nc(-c5ccc6ccccc6c5)nc(-c5cccc6sc7ccccc7c56)n4)c3)c3c2oc2ccccc23)cc1]

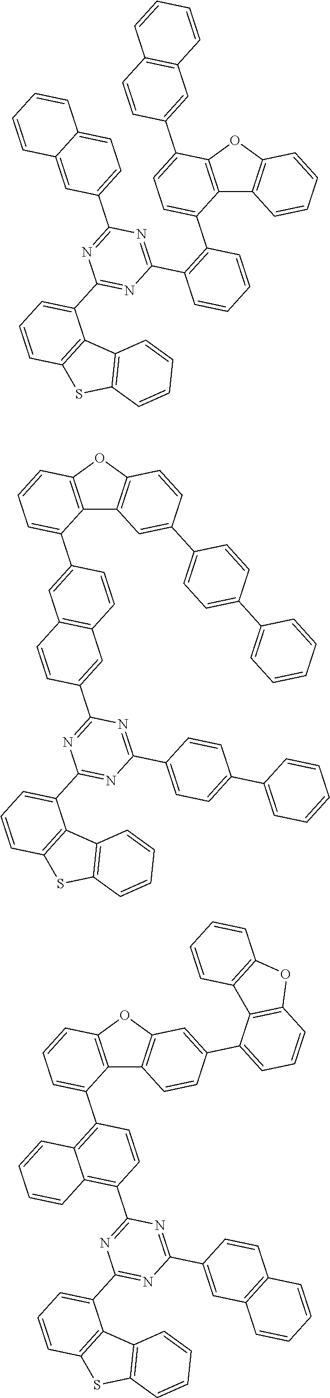 [SMILES: c1ccc(-c2ccc(-c3ccc4ccccc4c3)c3oc4ccccc4c23)c(-c2nc(-c3ccc4ccccc4c3)nc(-c3cccc4sc5ccccc5c34)n2)c1 ; c1ccc(-c2ccc(-c3ccc4oc5cccc(-c6ccc7cc(-c8nc(-c9ccc(-c%10ccccc%10)cc9)nc(-c9cccc%10sc%11ccccc%11c9%10)n8)ccc7c6)c5c4c3)cc2)cc1 ; c1ccc2cc(-c3nc(-c4ccc(-c5cccc6oc7cc(-c8cccc9oc%10ccccc%10c89)ccc7c56)c5ccccc45)nc(-c4cccc5sc6ccccc6c45)n3)ccc2c1]

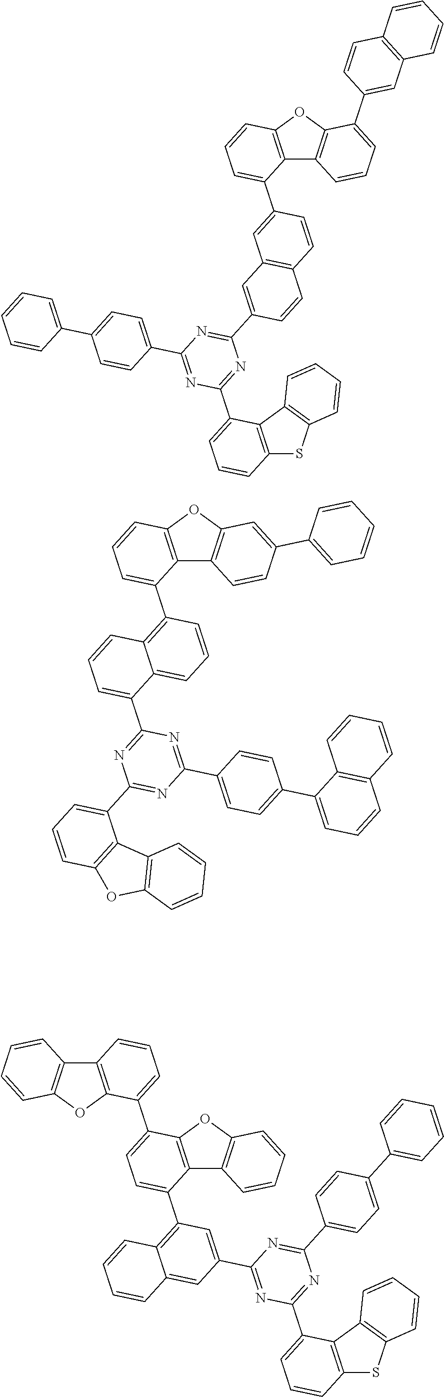 [SMILES: c1ccc(-c2ccc(-c3nc(-c4ccc5ccc(-c6cccc7oc8c(-c9ccc%10ccccc%10c9)cccc8c67)cc5c4)nc(-c4cccc5sc6ccccc6c45)n3)cc2)cc1 ; c1ccc(-c2ccc3c(c2)oc2cccc(-c4cccc5c(-c6nc(-c7ccc(-c8cccc9ccccc89)cc7)nc(-c7cccc8oc9ccccc9c78)n6)cccc45)c23)cc1 ; c1ccc(-c2ccc(-c3nc(-c4cc(-c5ccc(-c6cccc7c6oc6ccccc67)c6oc7ccccc7c56)c5ccccc5c4)nc(-c4cccc5sc6ccccc6c45)n3)cc2)cc1]

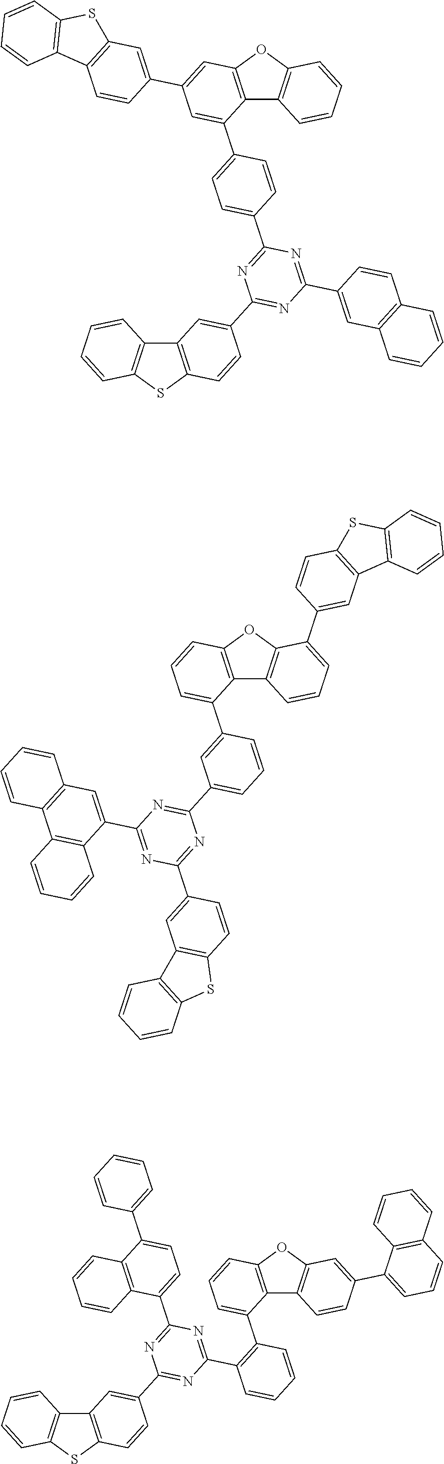 [SMILES: c1ccc2cc(-c3nc(-c4ccc(-c5cc(-c6ccc7c(c6)sc6ccccc67)cc6oc7ccccc7c56)cc4)nc(-c4ccc5sc6ccccc6c5c4)n3)ccc2c1 ; c1cc(-c2nc(-c3ccc4sc5ccccc5c4c3)nc(-c3cc4ccccc4c4ccccc34)n2)cc(-c2cccc3oc4c(-c5ccc6sc7ccccc7c6c5)cccc4c23)c1 ; c1ccc(-c2ccc(-c3nc(-c4ccc5sc6ccccc6c5c4)nc(-c4ccccc4-c4cccc5oc6cc(-c7cccc8ccccc78)ccc6c45)n3)c3ccccc23)cc1]

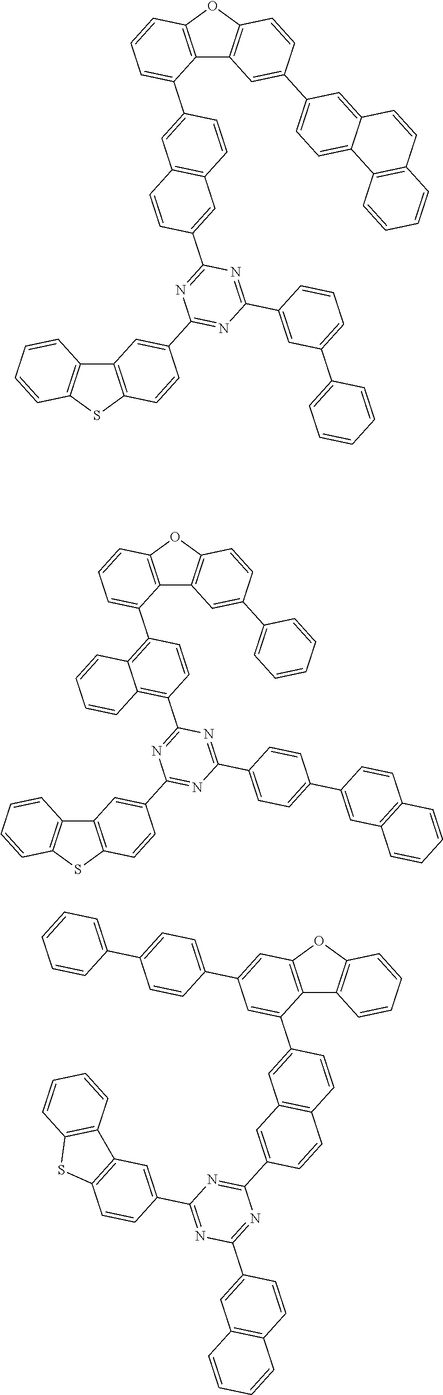

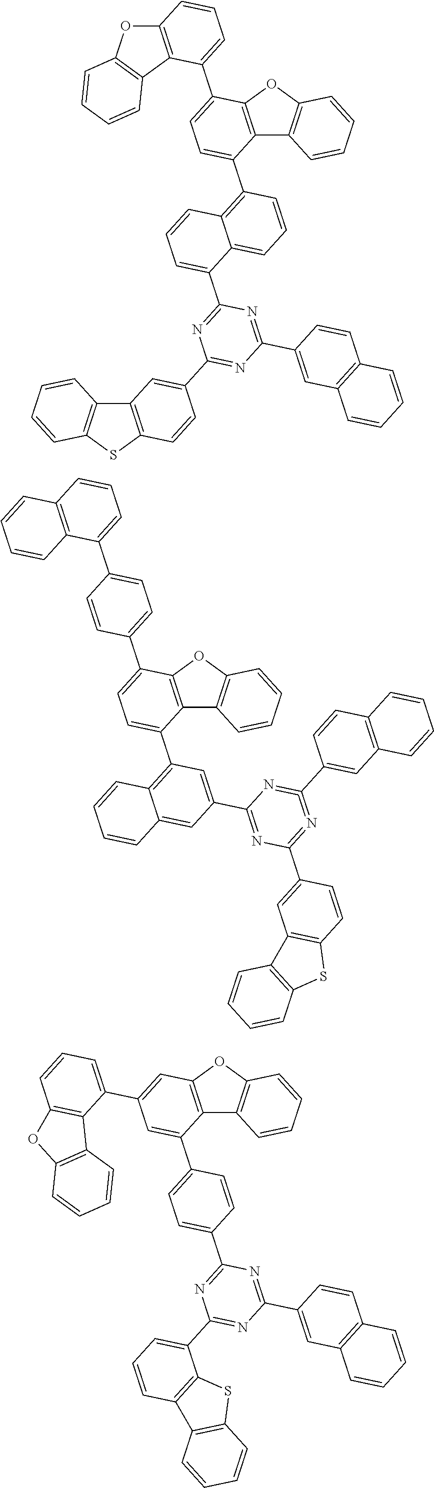

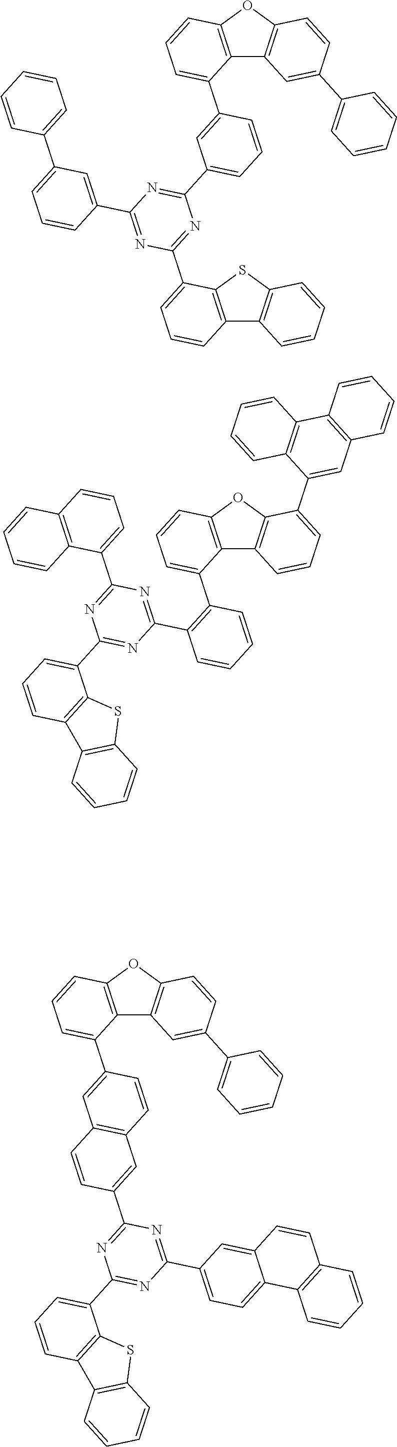 [SMILES: c1ccc(-c2cccc(-c3nc(-c4cccc(-c5cccc6oc7ccc(-c8ccccc8)cc7c56)c4)nc(-c4cccc5c4sc4ccccc45)n3)c2)cc1 ; c1ccc(-c2cccc3oc4c(-c5cc6ccccc6c6ccccc56)cccc4c23)c(-c2nc(-c3cccc4ccccc34)nc(-c3cccc4c3sc3ccccc34)n2)c1 ; c1ccc(-c2ccc3oc4cccc(-c5ccc6cc(-c7nc(-c8ccc9c(ccc%10ccccc%109)c8)nc(-c8cccc9c8sc8ccccc89)n7)ccc6c5)c4c3c2)cc1]

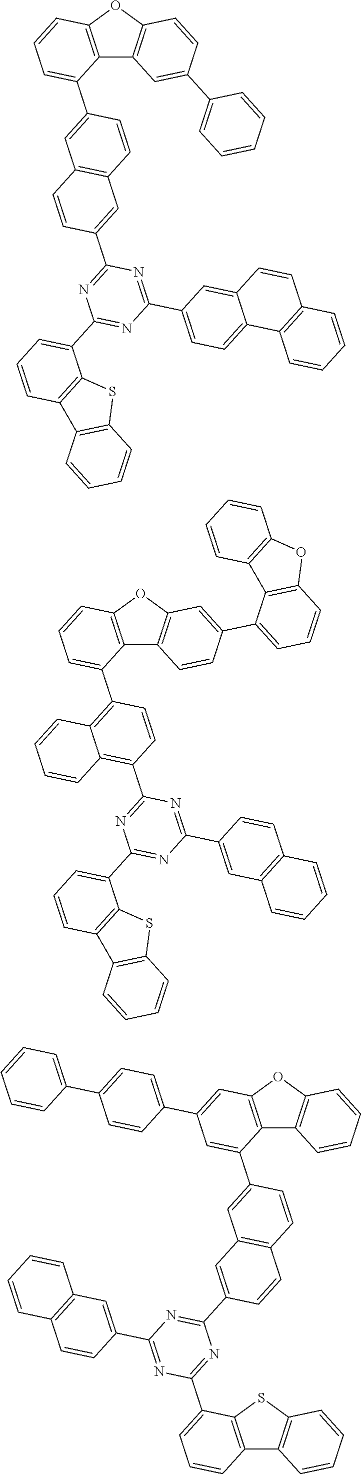 [SMILES: c1ccc(-c2ccc3oc4cccc(-c5ccc6cc(-c7nc(-c8ccc9c(ccc%10ccccc%109)c8)nc(-c8cccc9c8sc8ccccc89)n7)ccc6c5)c4c3c2)cc1 ; c1ccc2cc(-c3nc(-c4ccc(-c5cccc6oc7cc(-c8cccc9oc%10ccccc%10c89)ccc7c56)c5ccccc45)nc(-c4cccc5c4sc4ccccc45)n3)ccc2c1 ; c1ccc(-c2ccc(-c3cc(-c4ccc5ccc(-c6nc(-c7ccc8ccccc8c7)nc(-c7cccc8c7sc7ccccc78)n6)cc5c4)c4c(c3)oc3ccccc34)cc2)cc1]

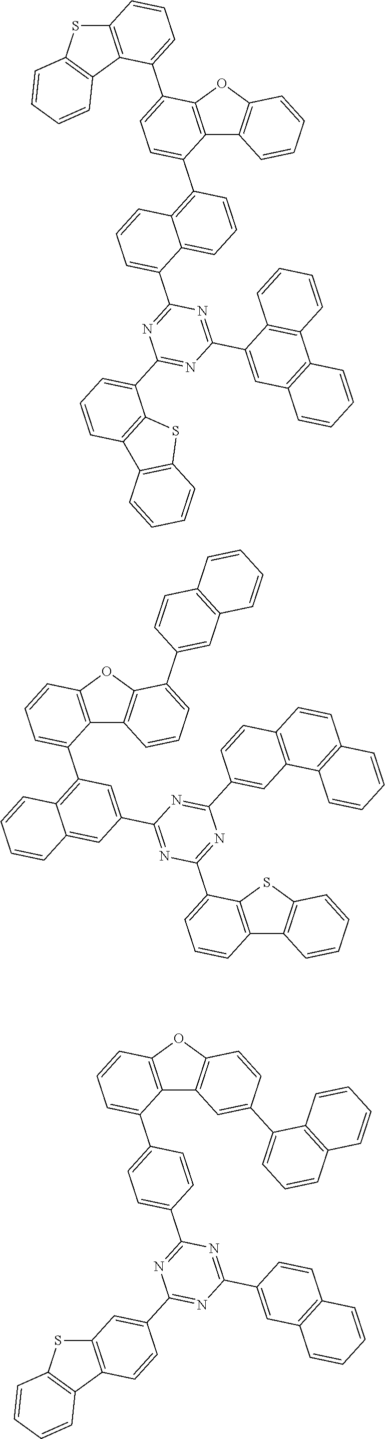

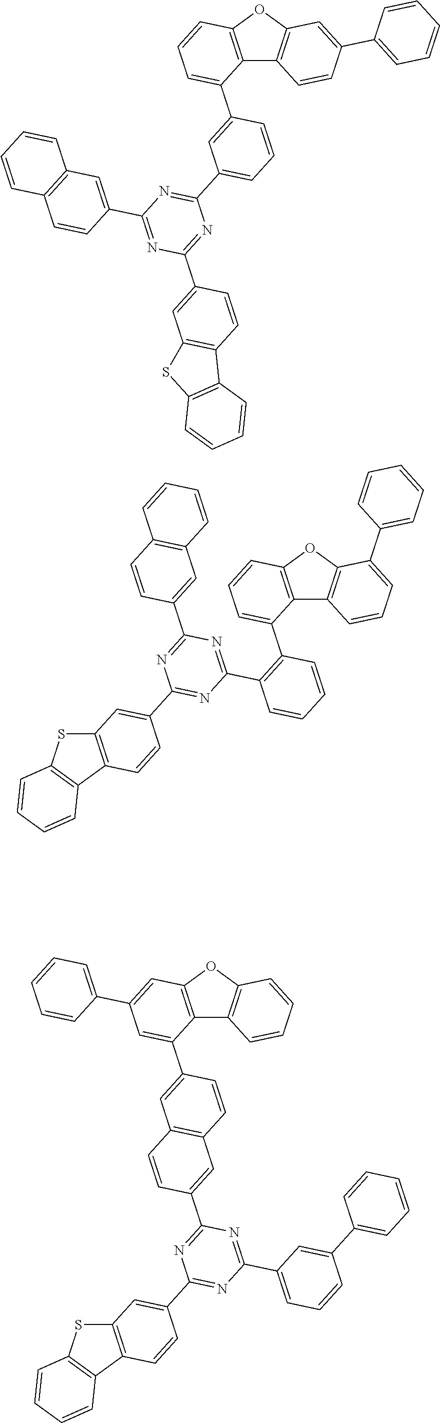

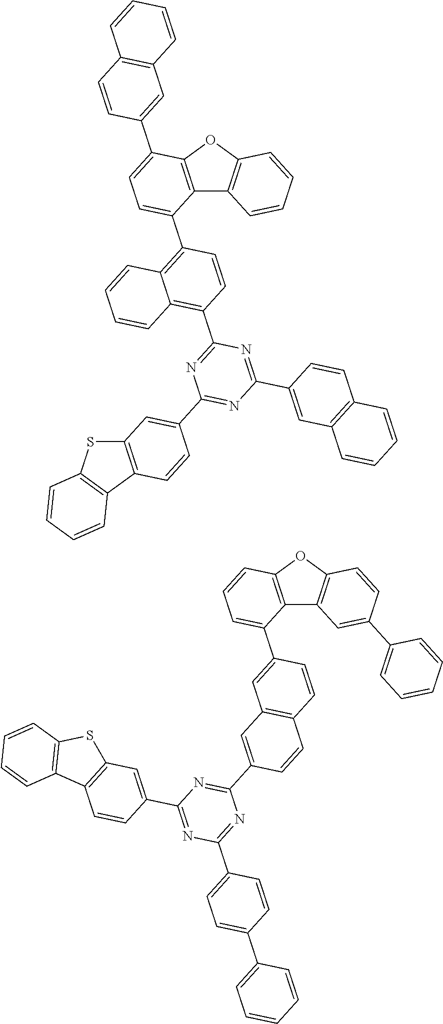 [SMILES: c1ccc2cc(-c3nc(-c4ccc5c(c4)sc4ccccc45)nc(-c4ccc(-c5ccc(-c6ccc7ccccc7c6)c6oc7ccccc7c56)c5ccccc45)n3)ccc2c1 ; c1ccc(-c2ccc(-c3nc(-c4ccc5ccc(-c6cccc7oc8ccc(-c9ccccc9)cc8c67)cc5c4)nc(-c4ccc5c(c4)sc4ccccc45)n3)cc2)cc1]

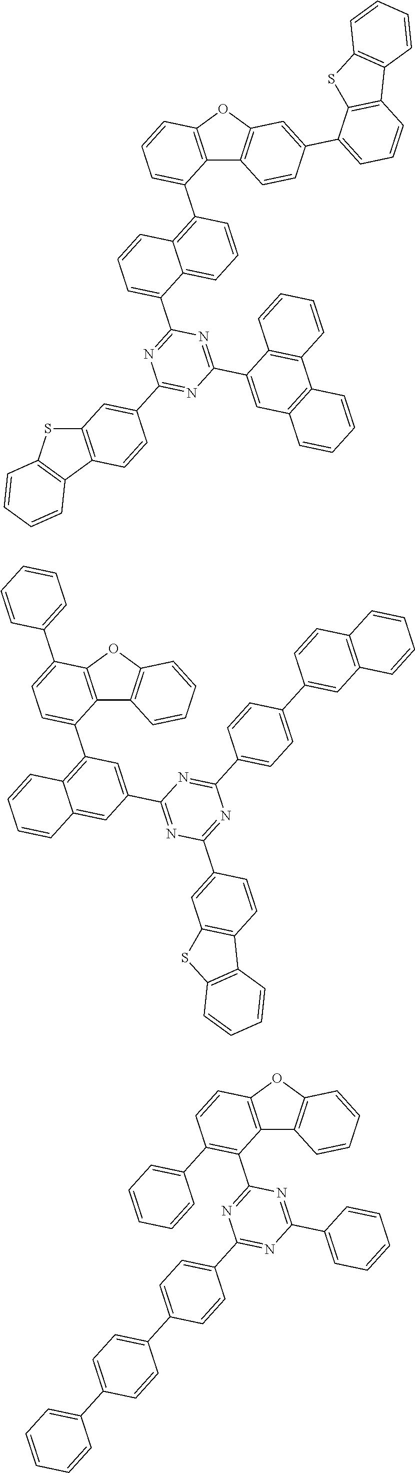

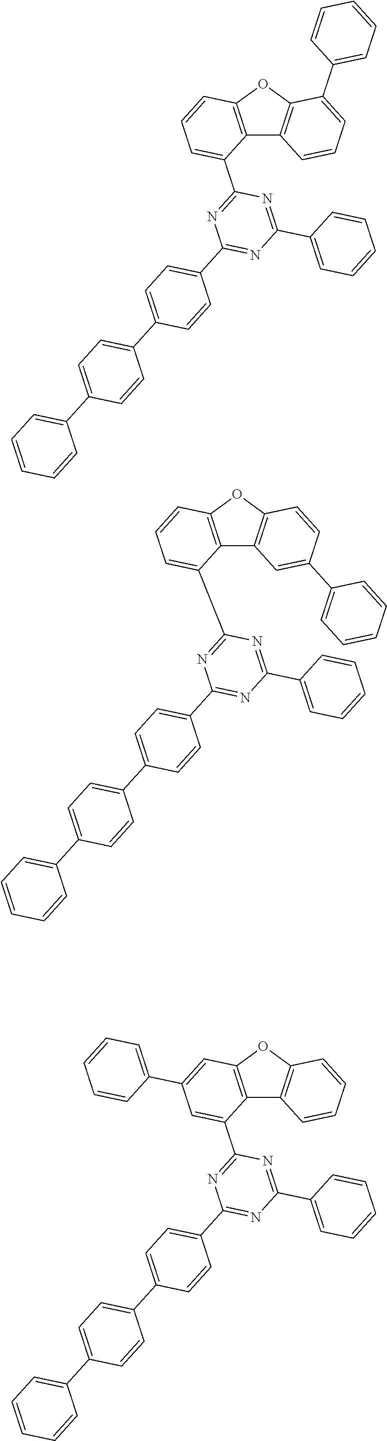 [SMILES: c1ccc(-c2ccc(-c3ccc(-c4nc(-c5ccccc5)nc(-c5cccc6oc7c(-c8ccccc8)cccc7c56)n4)cc3)cc2)cc1 ; c1ccc(-c2ccc(-c3ccc(-c4nc(-c5ccccc5)nc(-c5cccc6oc7ccc(-c8ccccc8)cc7c56)n4)cc3)cc2)cc1 ; c1ccc(-c2ccc(-c3ccc(-c4nc(-c5ccccc5)nc(-c5cc(-c6ccccc6)cc6oc7ccccc7c56)n4)cc3)cc2)cc1]

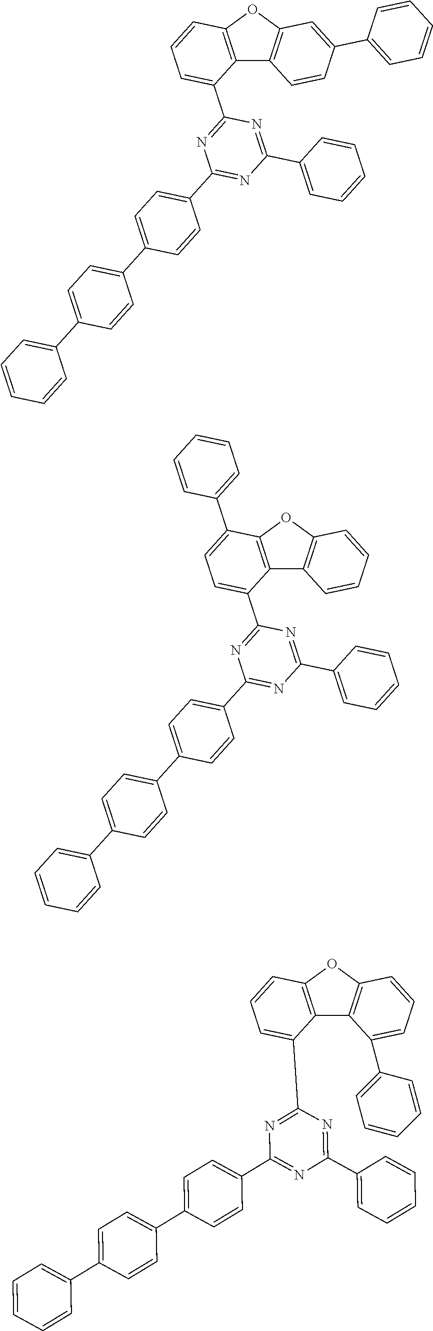 [SMILES: c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1]

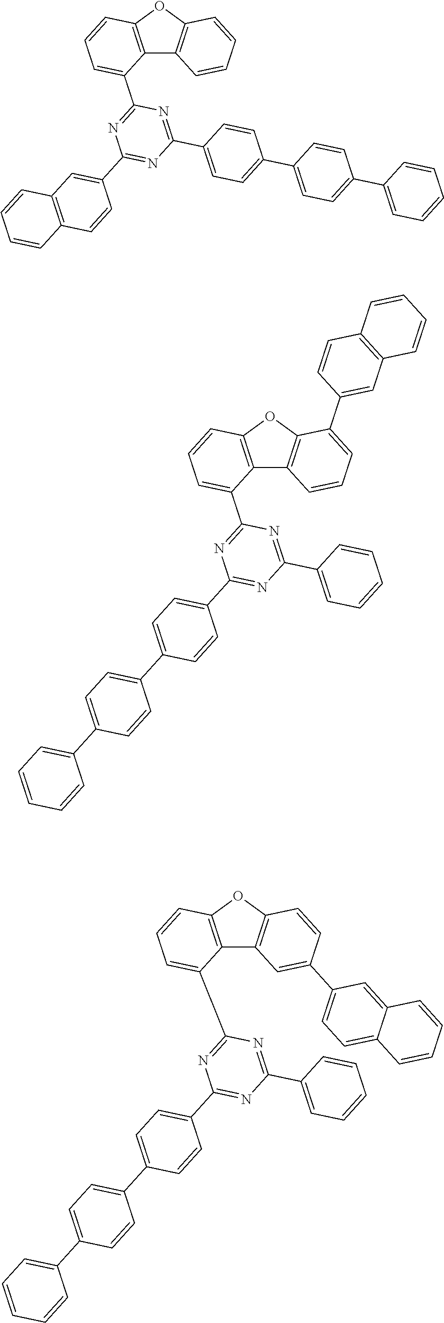 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1]

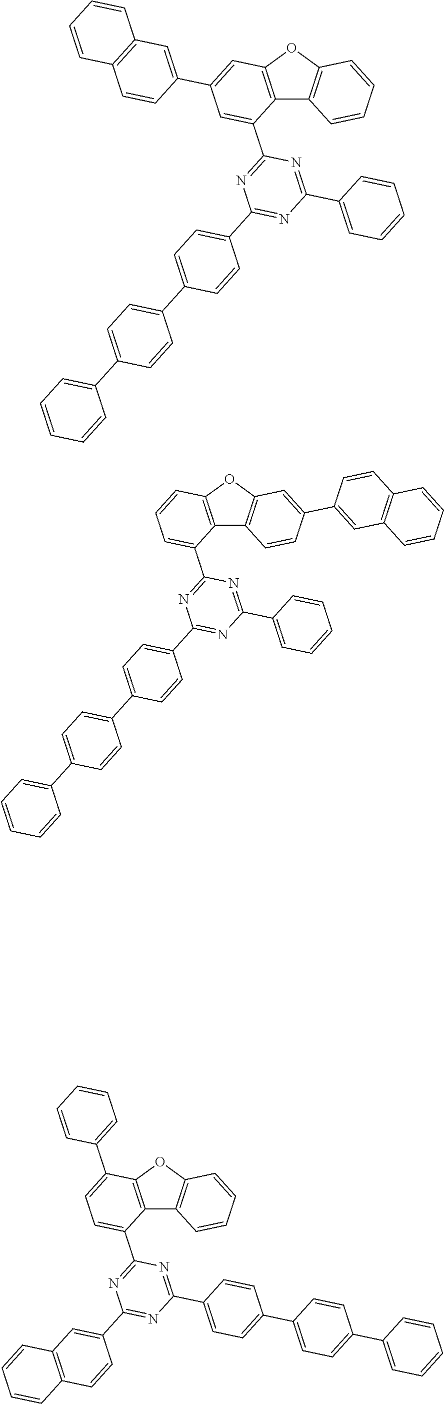 [SMILES: c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccc6ccccc6c5)nc(-c5ccc(-c6ccccc6)c6oc7ccccc7c56)n4)cc3)cc2)cc1]

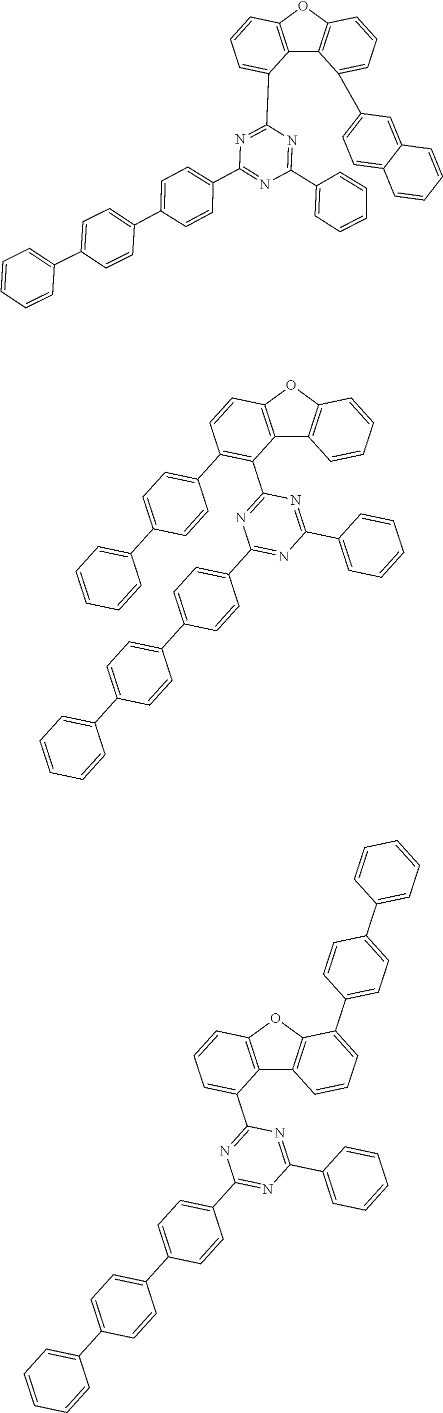 [SMILES: c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1]

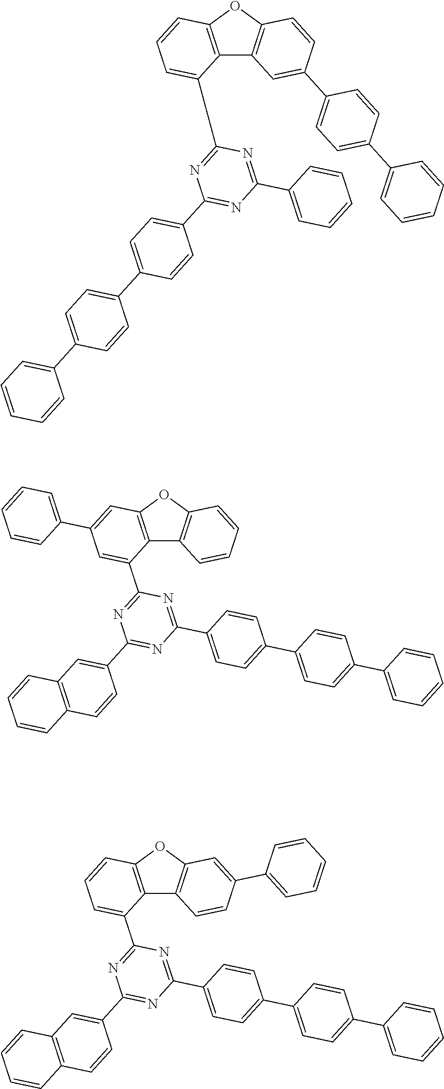 [SMILES: c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1 ; c1ccc(-c2ccc(-c3ccc(-c4nc(-c5ccc6ccccc6c5)nc(-c5cc(-c6ccccc6)cc6oc7ccccc7c56)n4)cc3)cc2)cc1 ; c1ccc(-c2ccc(-c3ccc(-c4nc(-c5ccc6ccccc6c5)nc(-c5cccc6oc7cc(-c8ccccc8)ccc7c56)n4)cc3)cc2)cc1]

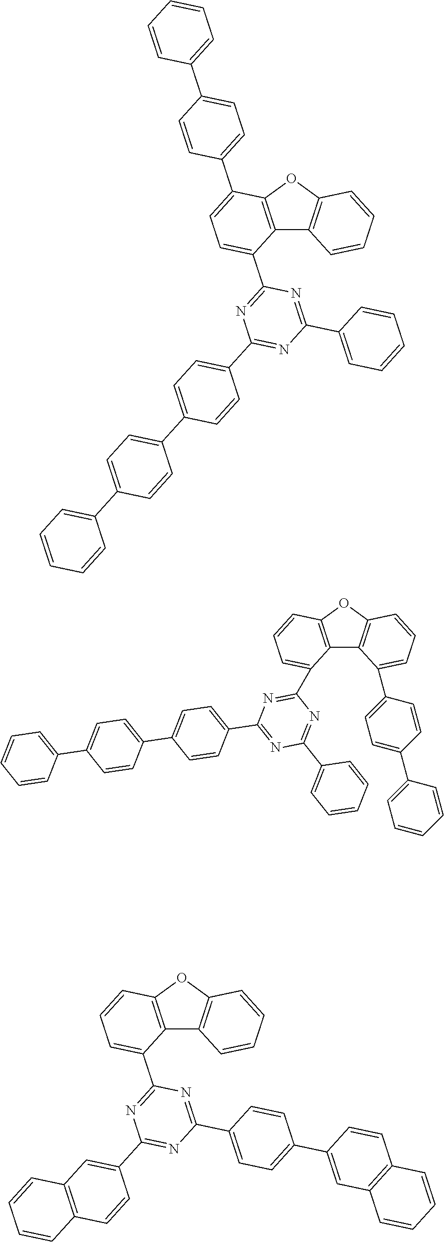 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

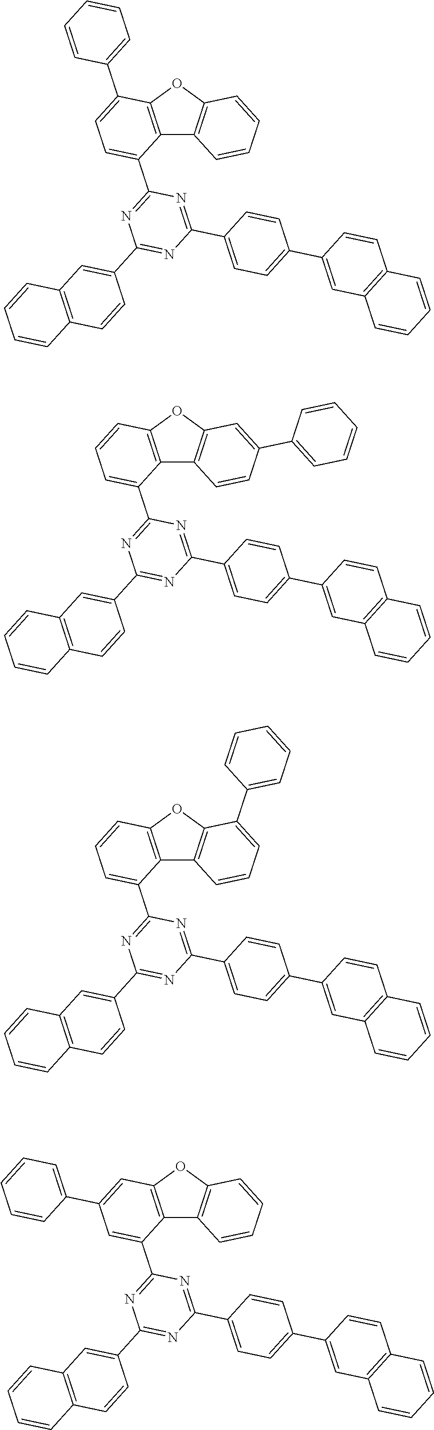 [SMILES: c1ccc(-c2ccc(-c3nc(-c4ccc(-c5ccc6ccccc6c5)cc4)nc(-c4ccc5ccccc5c4)n3)c3c2oc2ccccc23)cc1 ; c1ccc(-c2ccc3c(c2)oc2cccc(-c4nc(-c5ccc(-c6ccc7ccccc7c6)cc5)nc(-c5ccc6ccccc6c5)n4)c23)cc1 ; c1ccc(-c2cccc3c2oc2cccc(-c4nc(-c5ccc(-c6ccc7ccccc7c6)cc5)nc(-c5ccc6ccccc6c5)n4)c23)cc1 ; c1ccc(-c2cc(-c3nc(-c4ccc(-c5ccc6ccccc6c5)cc4)nc(-c4ccc5ccccc5c4)n3)c3c(c2)oc2ccccc23)cc1]

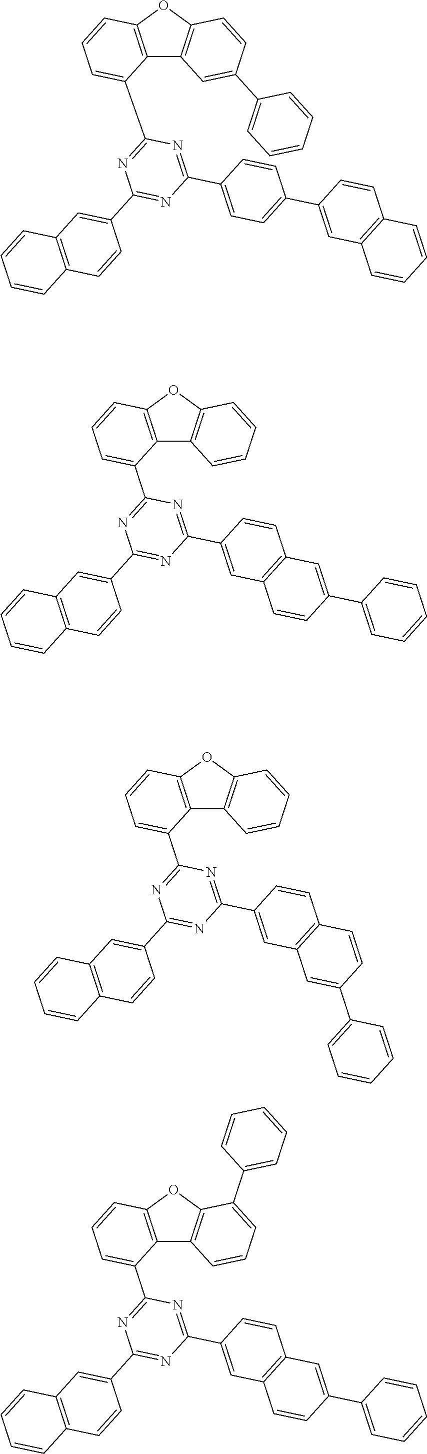 [SMILES: c1ccc(-c2ccc3oc4cccc(-c5nc(-c6ccc(-c7ccc8ccccc8c7)cc6)nc(-c6ccc7ccccc7c6)n5)c4c3c2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1 ; c1ccc(-c2ccc3cc(-c4nc(-c5ccc6ccccc6c5)nc(-c5cccc6oc7c(-c8ccccc8)cccc7c56)n4)ccc3c2)cc1]

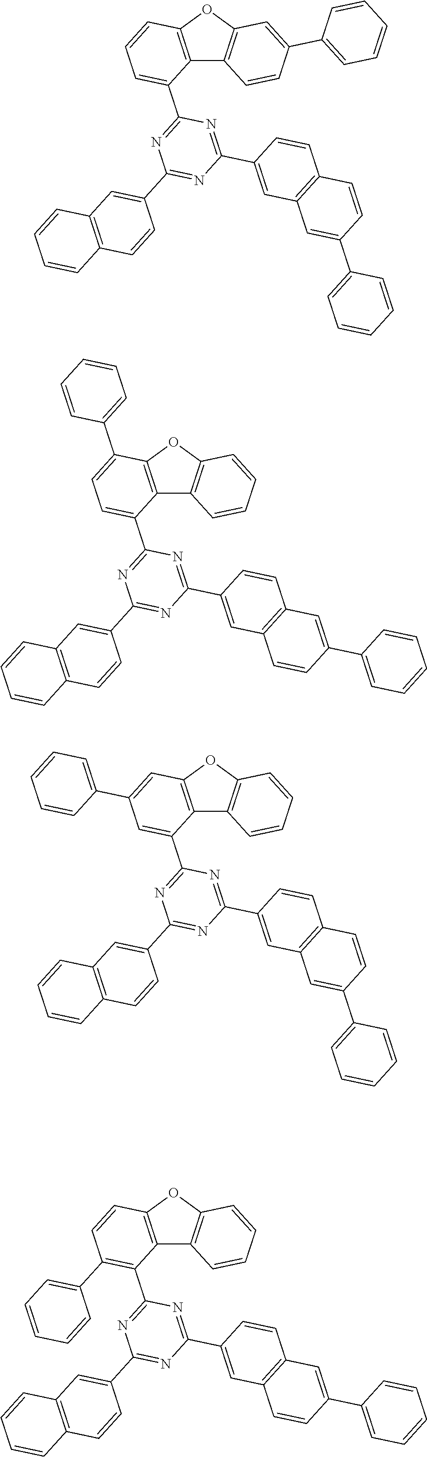 [SMILES: c1ccc(-c2ccc3ccc(-c4nc(-c5ccc6ccccc6c5)nc(-c5cccc6oc7cc(-c8ccccc8)ccc7c56)n4)cc3c2)cc1 ; c1ccc(-c2ccc3cc(-c4nc(-c5ccc6ccccc6c5)nc(-c5ccc(-c6ccccc6)c6oc7ccccc7c56)n4)ccc3c2)cc1 ; c1ccc(-c2ccc3ccc(-c4nc(-c5ccc6ccccc6c5)nc(-c5cc(-c6ccccc6)cc6oc7ccccc7c56)n4)cc3c2)cc1 ; c1ccc(-c2ccc3cc(-c4nc(-c5ccc6ccccc6c5)nc(-c5c(-c6ccccc6)ccc6oc7ccccc7c56)n4)ccc3c2)cc1]

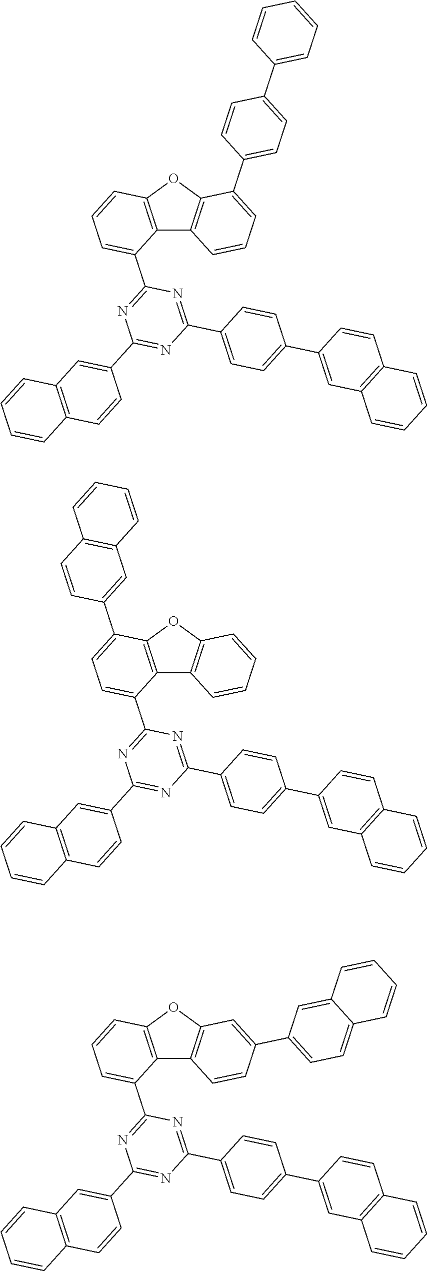 [SMILES: c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1 ; c1ccc2cc(-c3ccc(-c4nc(-c5ccc6ccccc6c5)nc(-c5cccc6oc7cc(-c8ccc9ccccc9c8)ccc7c56)n4)cc3)ccc2c1]

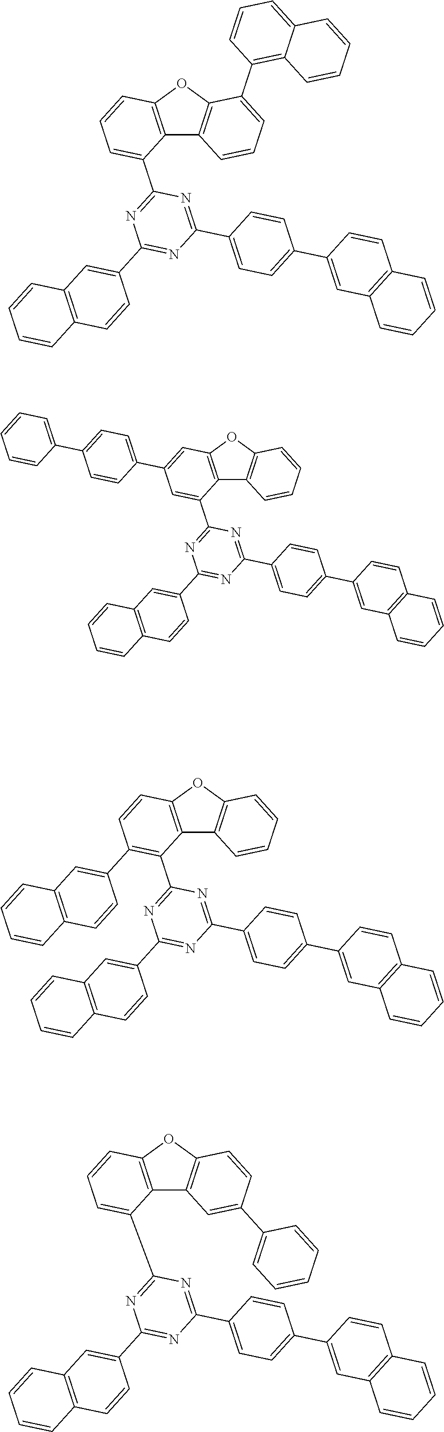 [SMILES: c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc(-c2ccc(-c3cc(-c4nc(-c5ccc(-c6ccc7ccccc7c6)cc5)nc(-c5ccc6ccccc6c5)n4)c4c(c3)oc3ccccc34)cc2)cc1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1 ; c1ccc(-c2ccc3oc4cccc(-c5nc(-c6ccc(-c7ccc8ccccc8c7)cc6)nc(-c6ccc7ccccc7c6)n5)c4c3c2)cc1]

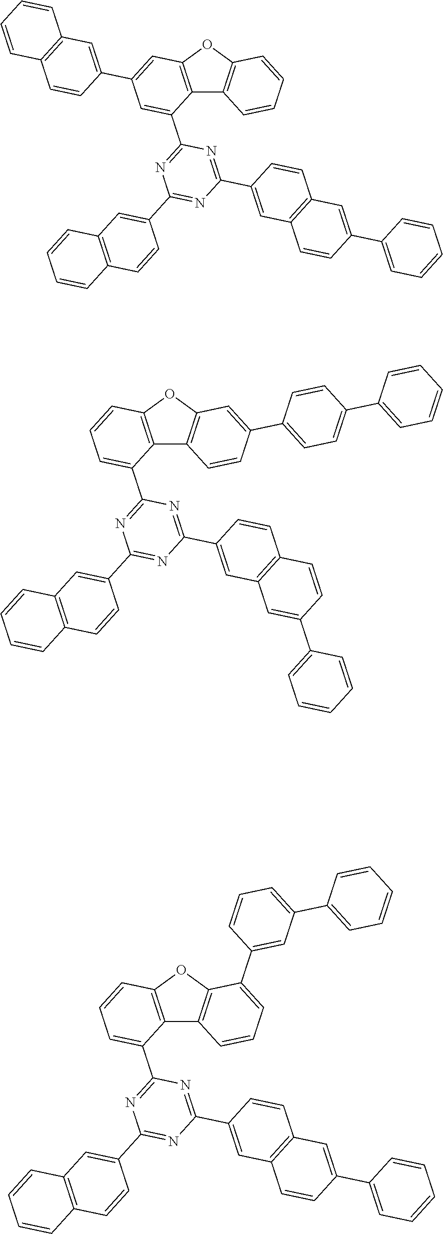 [SMILES: c1ccc(-c2ccc3cc(-c4nc(-c5ccc6ccccc6c5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccc7ccccc7c6)nc(-c6ccc7ccc(-c8ccccc8)cc7c6)n5)c34)cc2)cc1 ; c1ccc(-c2cccc(-c3cccc4c3oc3cccc(-c5nc(-c6ccc7ccccc7c6)nc(-c6ccc7cc(-c8ccccc8)ccc7c6)n5)c34)c2)cc1]

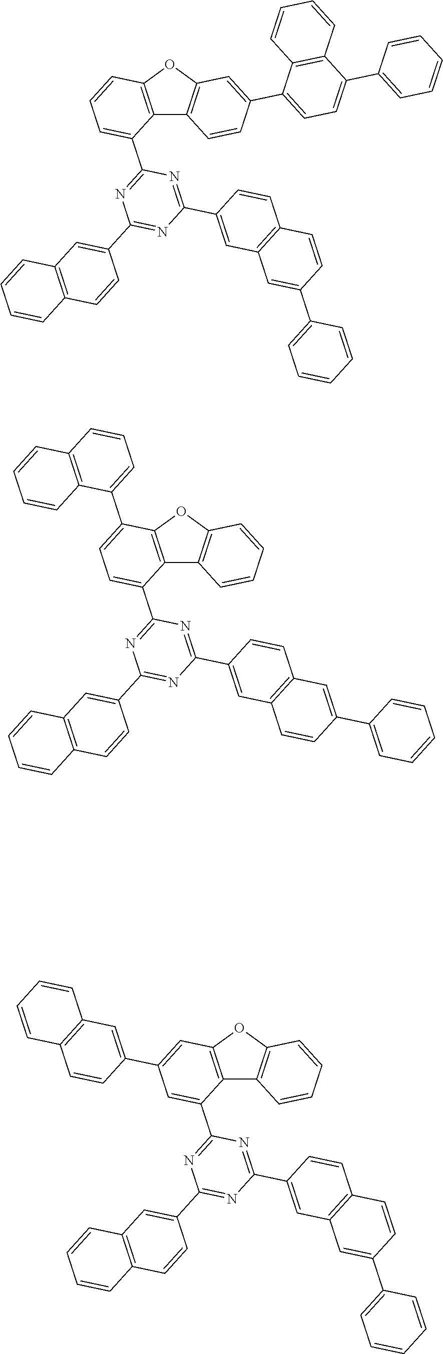 [SMILES: c1ccc(-c2ccc3ccc(-c4nc(-c5ccc6ccccc6c5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccc6ccccc6c5)nc(-c5ccc(-c6cccc7ccccc67)c6oc7ccccc7c56)n4)ccc3c2)cc1 ; c1ccc(-c2ccc3ccc(-c4nc(-c5ccc6ccccc6c5)nc(-c5cc(-c6ccc7ccccc7c6)cc6oc7ccccc7c56)n4)cc3c2)cc1]

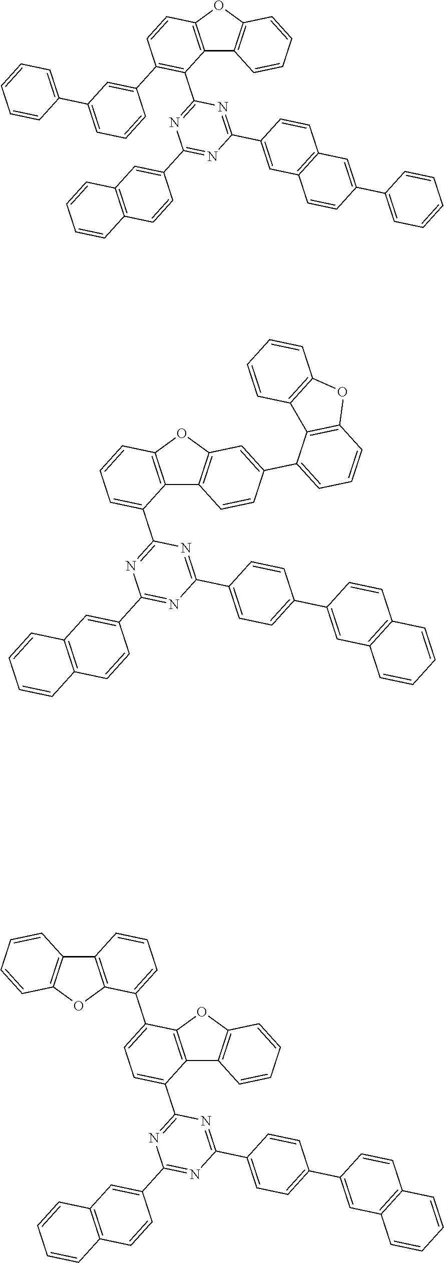 [SMILES: c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1 ; c1ccc2cc(-c3ccc(-c4nc(-c5ccc6ccccc6c5)nc(-c5cccc6oc7cc(-c8cccc9oc%10ccccc%10c89)ccc7c56)n4)cc3)ccc2c1 ; c1ccc2cc(-c3ccc(-c4nc(-c5ccc6ccccc6c5)nc(-c5ccc(-c6cccc7c6oc6ccccc67)c6oc7ccccc7c56)n4)cc3)ccc2c1]

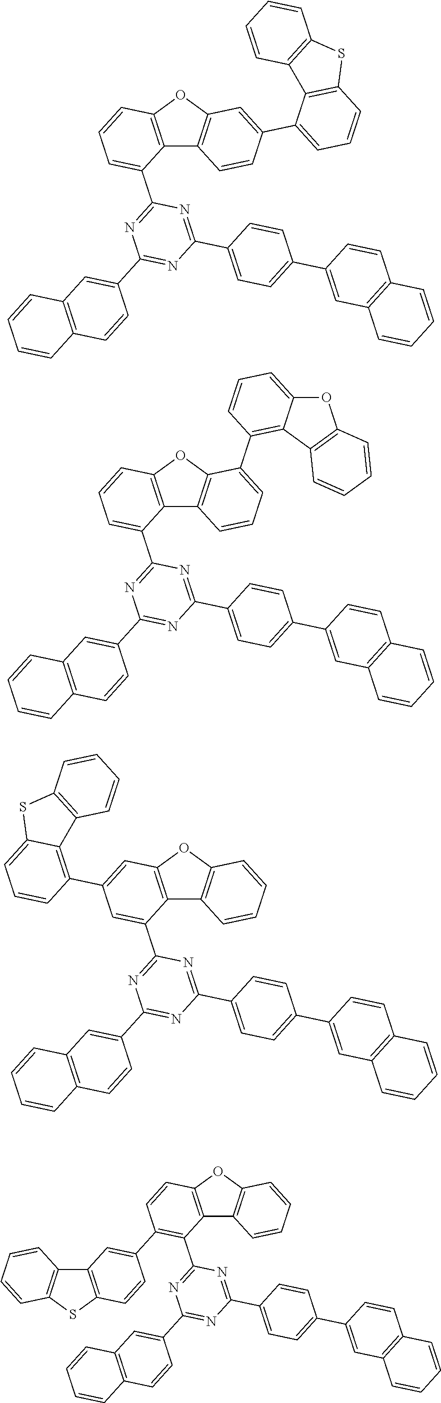

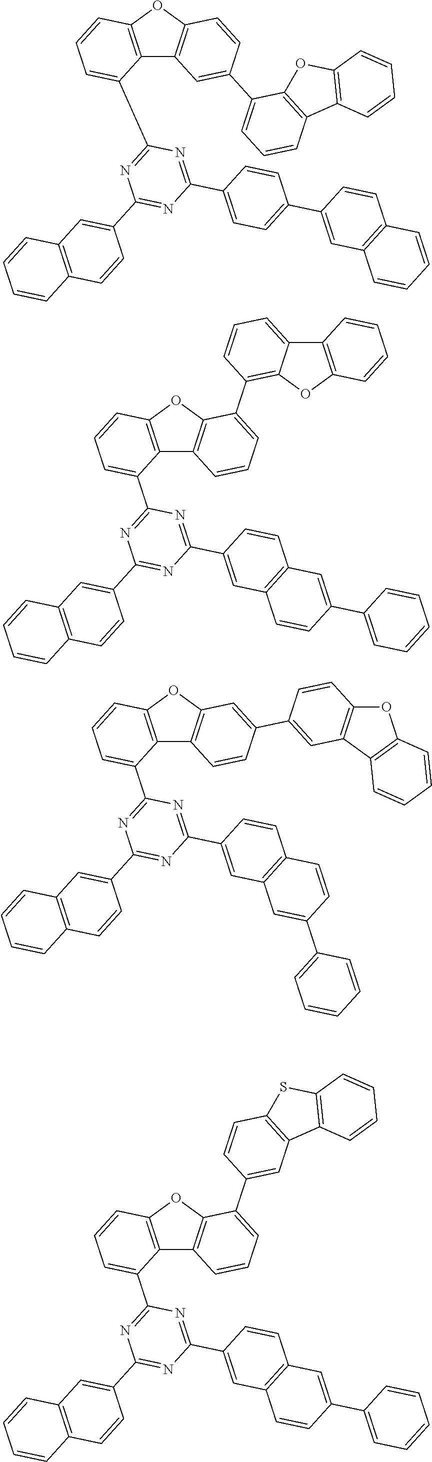

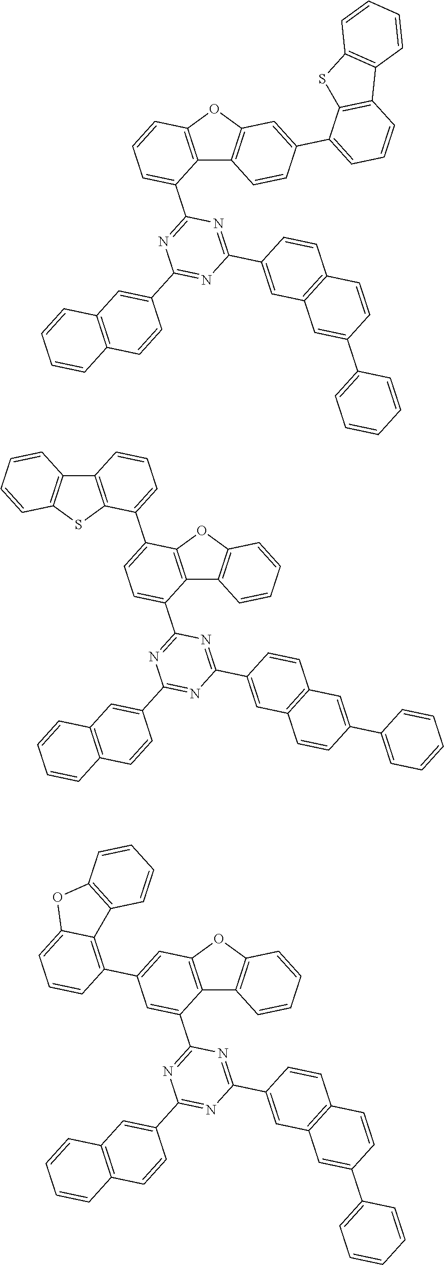 [SMILES: c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1 ; c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1 ; c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1]

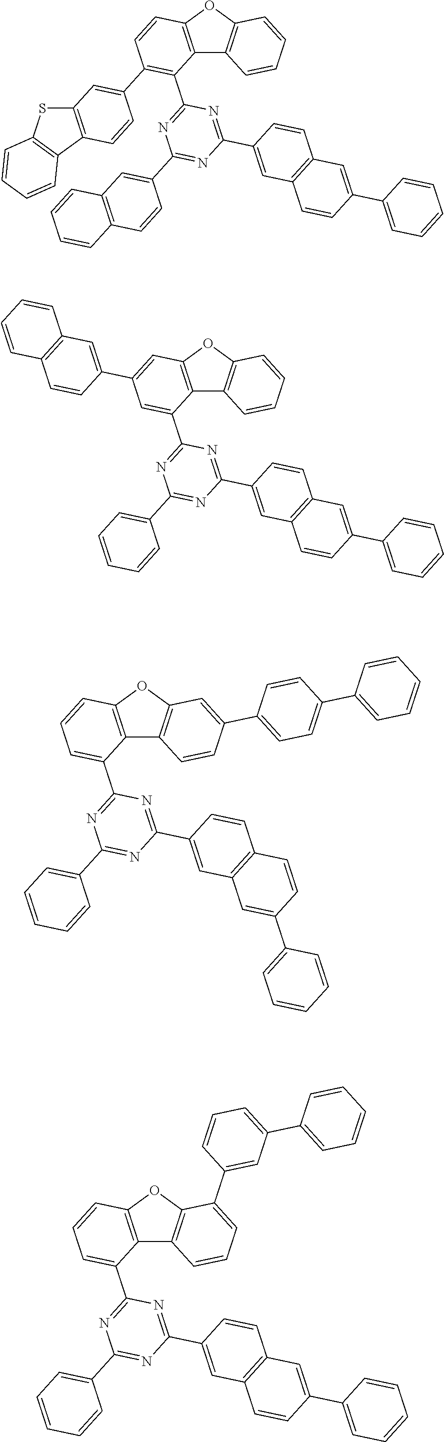 [SMILES: c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccccc6)nc(-c6ccc7ccc(-c8ccccc8)cc7c6)n5)c34)cc2)cc1 ; c1ccc(-c2cccc(-c3cccc4c3oc3cccc(-c5nc(-c6ccccc6)nc(-c6ccc7cc(-c8ccccc8)ccc7c6)n5)c34)c2)cc1]

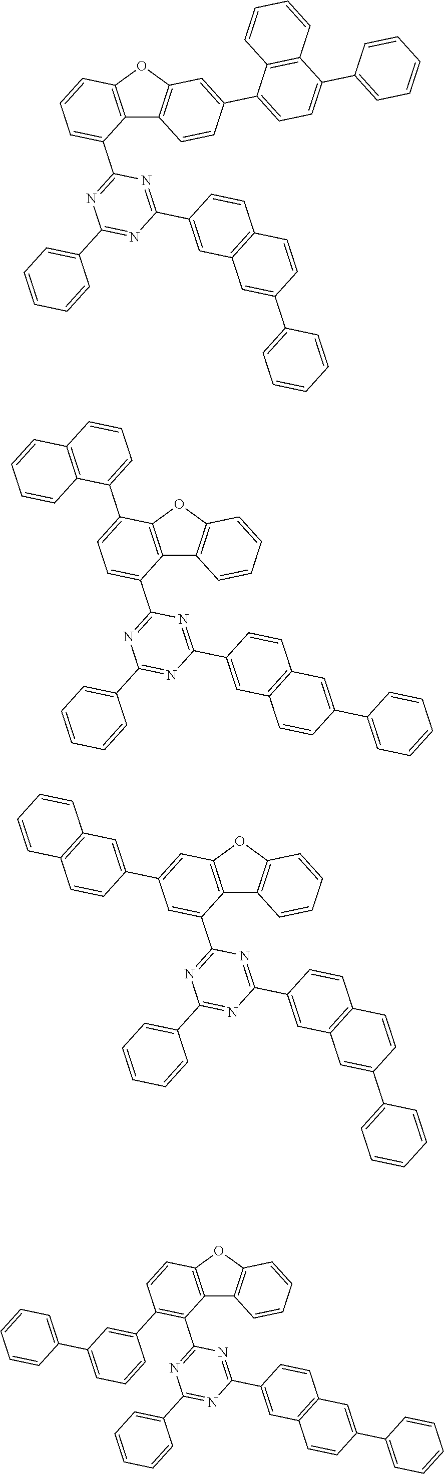

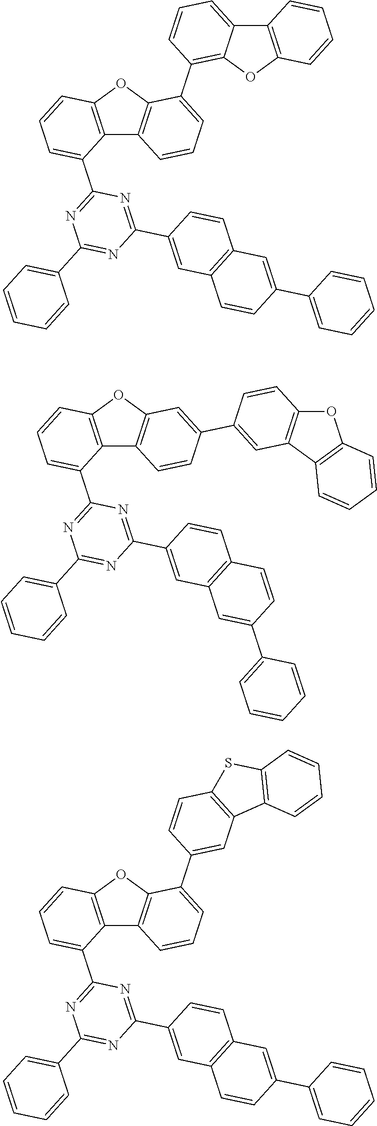

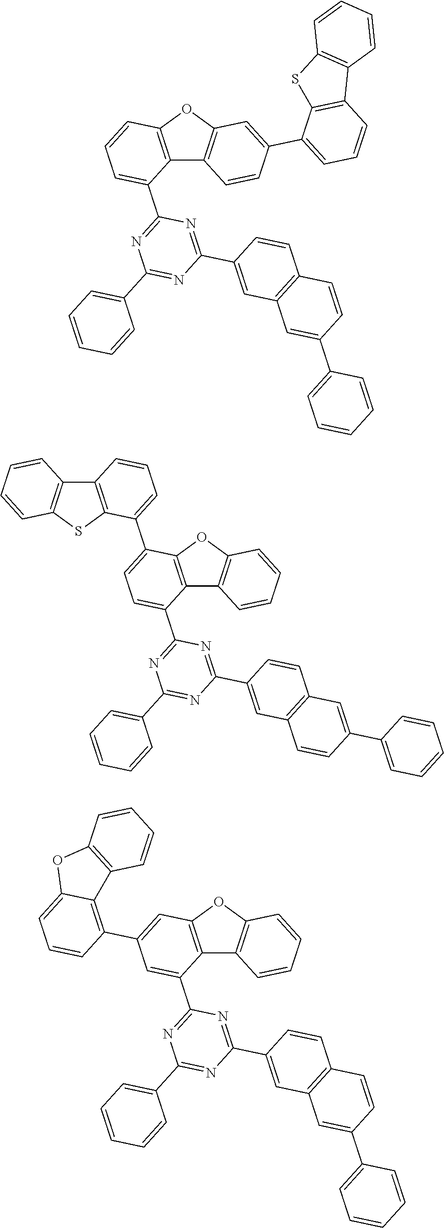

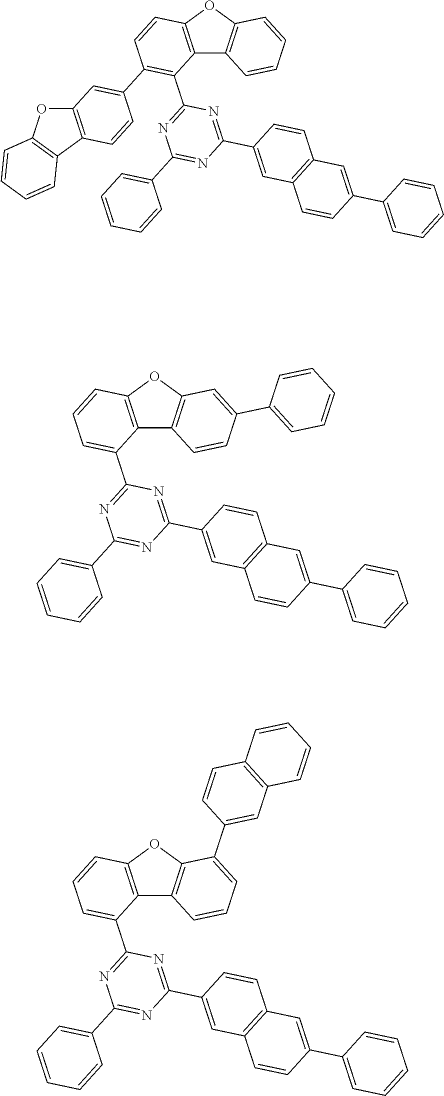 [SMILES: c1ccc(-c2ccc3cc(-c4nc(-c5ccccc5)nc(-c5c(-c6ccc7c(c6)oc6ccccc67)ccc6oc7ccccc7c56)n4)ccc3c2)cc1 ; c1ccc(-c2ccc3cc(-c4nc(-c5ccccc5)nc(-c5cccc6oc7cc(-c8ccccc8)ccc7c56)n4)ccc3c2)cc1 ; c1ccc(-c2ccc3cc(-c4nc(-c5ccccc5)nc(-c5cccc6oc7c(-c8ccc9ccccc9c8)cccc7c56)n4)ccc3c2)cc1]

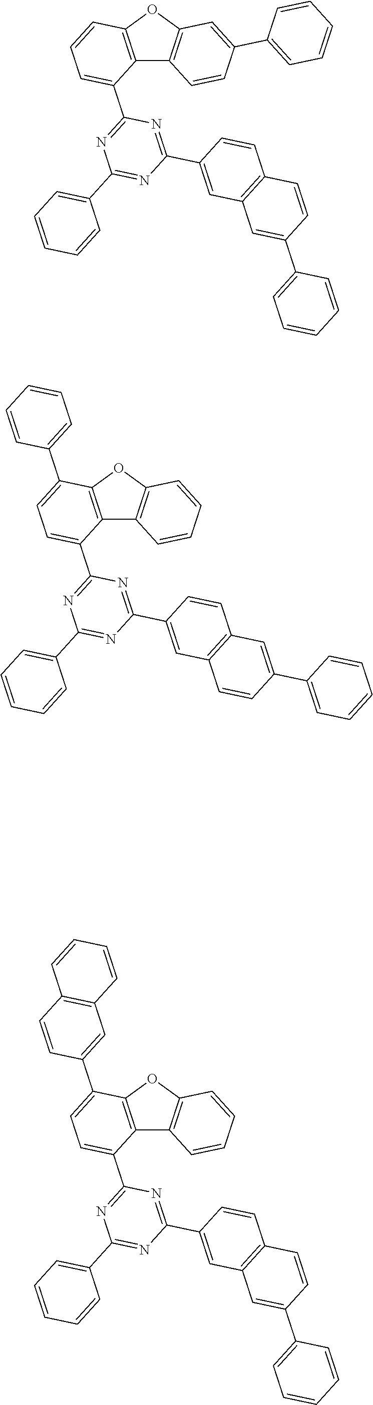 [SMILES: c1ccc(-c2ccc3ccc(-c4nc(-c5ccccc5)nc(-c5cccc6oc7cc(-c8ccccc8)ccc7c56)n4)cc3c2)cc1 ; c1ccc(-c2ccc3cc(-c4nc(-c5ccccc5)nc(-c5ccc(-c6ccccc6)c6oc7ccccc7c56)n4)ccc3c2)cc1 ; c1ccc(-c2ccc3ccc(-c4nc(-c5ccccc5)nc(-c5ccc(-c6ccc7ccccc7c6)c6oc7ccccc7c56)n4)cc3c2)cc1]

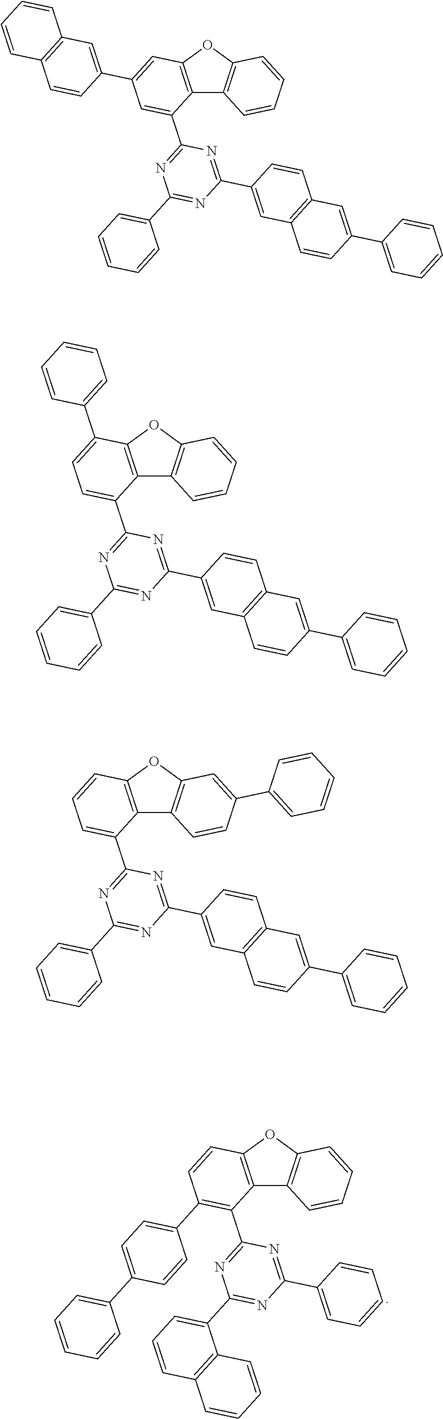 [SMILES: c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2ccc3cc(-c4nc(-c5ccccc5)nc(-c5ccc(-c6ccccc6)c6oc7ccccc7c56)n4)ccc3c2)cc1 ; c1ccc(-c2ccc3cc(-c4nc(-c5ccccc5)nc(-c5cccc6oc7cc(-c8ccccc8)ccc7c56)n4)ccc3c2)cc1 ; c1ccc(-c2ccc(-c3ccc4oc5ccccc5c4c3-c3nc(-c4ccccc4)nc(-c4cccc5ccccc45)n3)cc2)cc1]

The compound of Chemical Formula 1 can be prepared, for example, according to the preparation method as shown in the following Reaction Scheme 1, an d the other remaining compounds can be prepared in a similar manner.

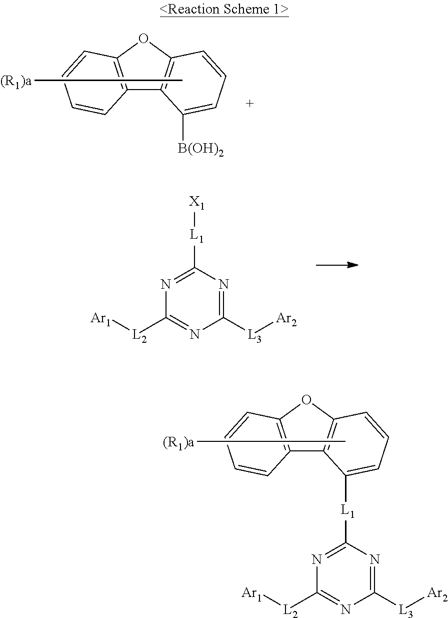

wherein in Reaction Scheme 1, Ar₁, Ar₂, L₁ to L₃, R₁ and a are as defined in Chemical Formula 1, and X₁ is halogen, preferably X₁ is chloro or bromo.

The Reaction Scheme 1 is a Suzuki coupling reaction, which is preferably carried out in the presence of a palladium catalyst and a base, and a reactive group for the Suzuki coupling reaction can be modified as known in the art. The above preparation method can be further embodied in the Preparation Examples described hereinafter.

Preferably, Ar₃ can be hydrogen, a substituted or unsubstituted C₆₋₂₀ aryl, or a substituted or unsubstituted C₂₋₂₀ heteroaryl containing one or more selected from the group consisting of N, O and S, and

more preferably, Ar₃ can be hydrogen or phenyl.

Preferably, Ar₄ and Ar₅ can be each independently a substituted or unsubstituted C₆₋₂₀ aryl or a substituted or unsubstituted C₂₋₂₀ heteroaryl containing one or more selected from the group consisting of N, O and S,

more preferably, Ar₄ and Ar₅ can be each independently phenyl, phenyl substituted with 5 deuteriums, biphenylyl, biphenylyl substituted with 4 deuteriums, biphenylyl substituted with 9 deuteriums, terphenylyl, terphenylyl substituted with 4 deuteriums, quaterphenylyl, naphthyl, phenanthrenyl, triphenylenyl, dimethylfluorenyl, diphenylfluorenyl, carbazolyl, phenylcarbazolyl, dibenzofuranyl, dibenzothiophenyl, or phenyl dibenzofuranyl, and

most preferably, Ar₄ and Ar₅ can be each independently any one selected from the group consisting of the following:

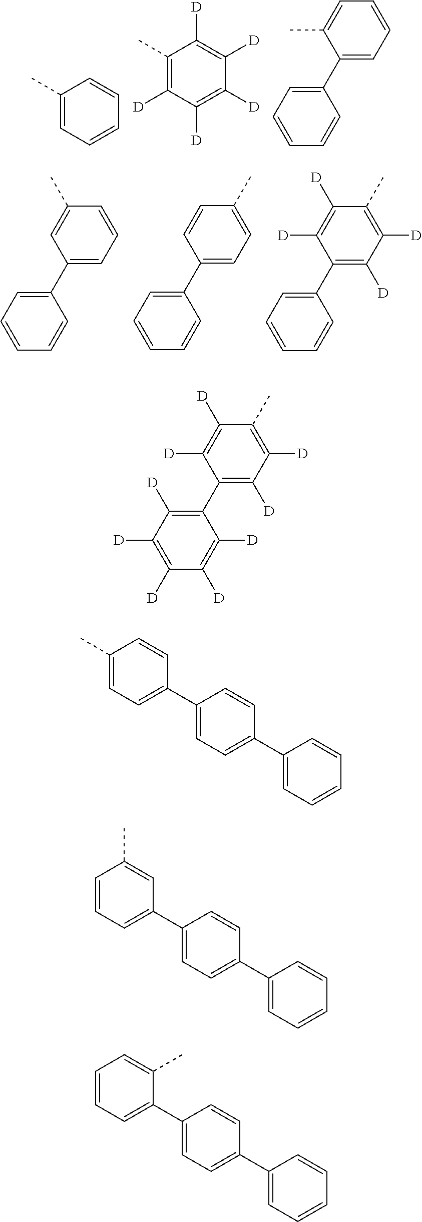

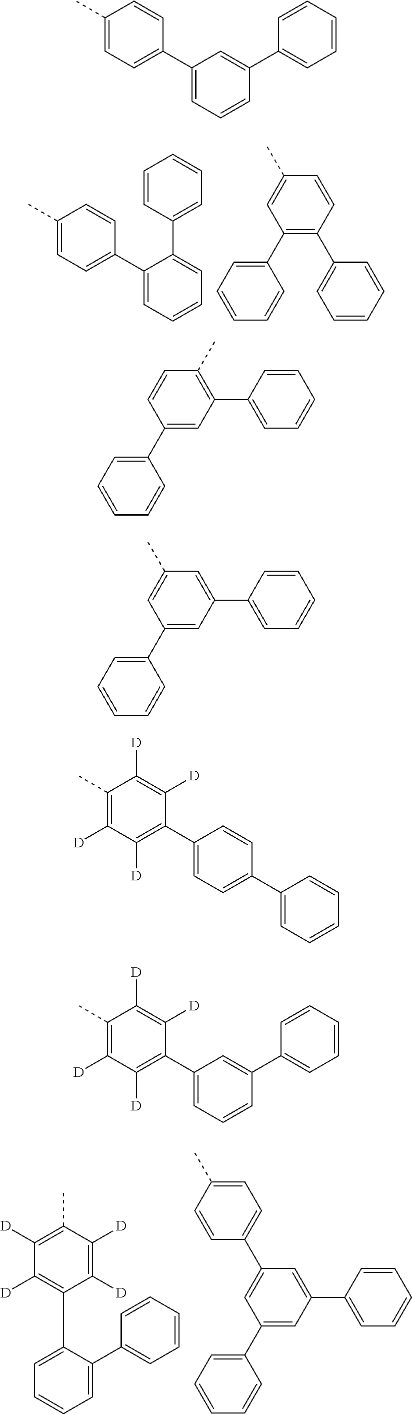

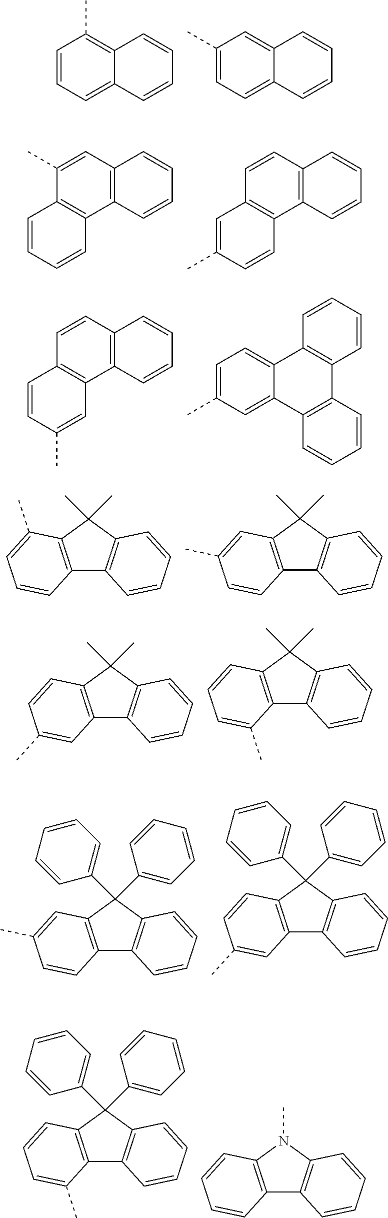

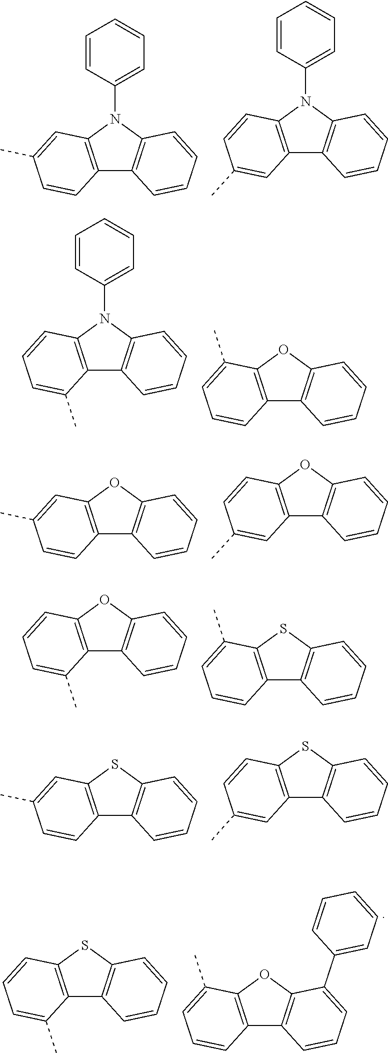

Preferably L₄ to L₆ can be each independently a single bond, a substituted or unsubstituted C₆₋₂₀ arylene, or a substituted or unsubstituted C₂₋₂₀ heteroarylene containing one or more selected from the group consisting of N, O and S,

more preferably, L₄ to L₆ can be each independently a single bond, phenylene, phenylene substituted with 4 deuteriums, biphenylylene, naphthylene, phenyl naphthylene, carbazolylene, phenyl carbazolylene, phenyl carbazolylene substituted with 4 deuteriums, dibenzofuranylene, phenyl dibenzofuranylene, phenyl dibenzofuranylene substituted with 4 deuteriums, or dimethylfluorenylene, and

most preferably, L₄ to L₆ can be each independently a single bond or any one selected from the group consisting of the following:

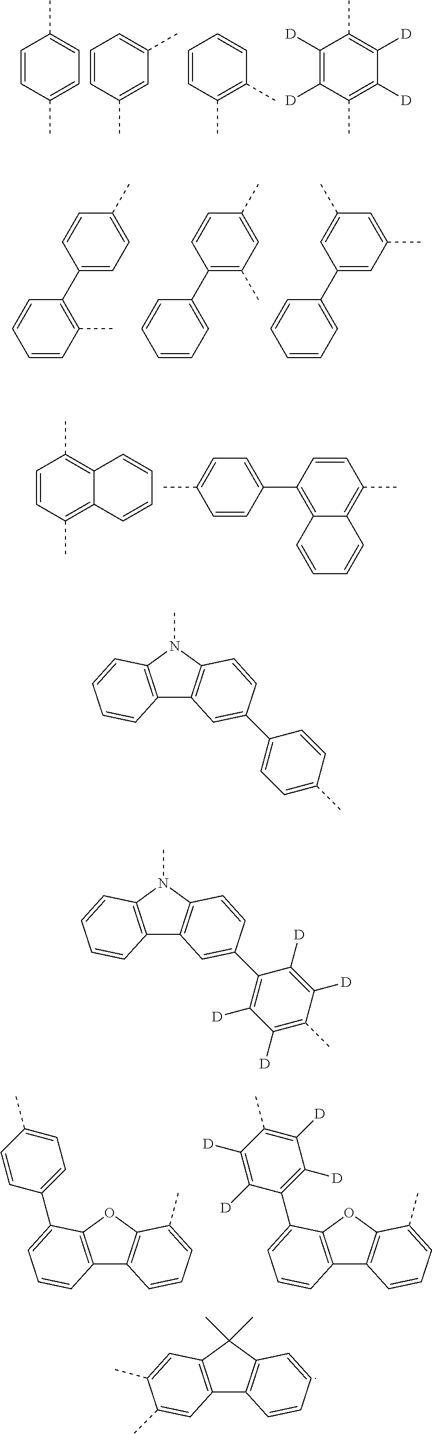

Preferably, L₄ is a single bond, and L₅ and L₆ can be each independently a single bond, a substituted or unsubstituted C₆₋₂₀ arylene, or a substituted or unsubstituted C₂₋₂₀ heteroarylene containing at least one selected from the group consisting of N, O and S,

more preferably, L₄ is a single bond, and L and L₆ can be each independently a single bond, phenylene, phenylene substituted with 4 deuteriums, biphenylylene, naphthylene, phenyl naphthylene, carbazolylene, phenyl carbazolylene, phenyl carbazolylene substituted with 4 deuteriums, dibenzofuranylene, phenyl dibenzofuranylene, phenyl dibenzofuranylene substituted with 4 deuteriums, or dimethylfluorenylene, and

most preferably, L₄ is a single bond, and L₅ and L₆ can be each independently a single bond or any one selected from the group consisting of the following:

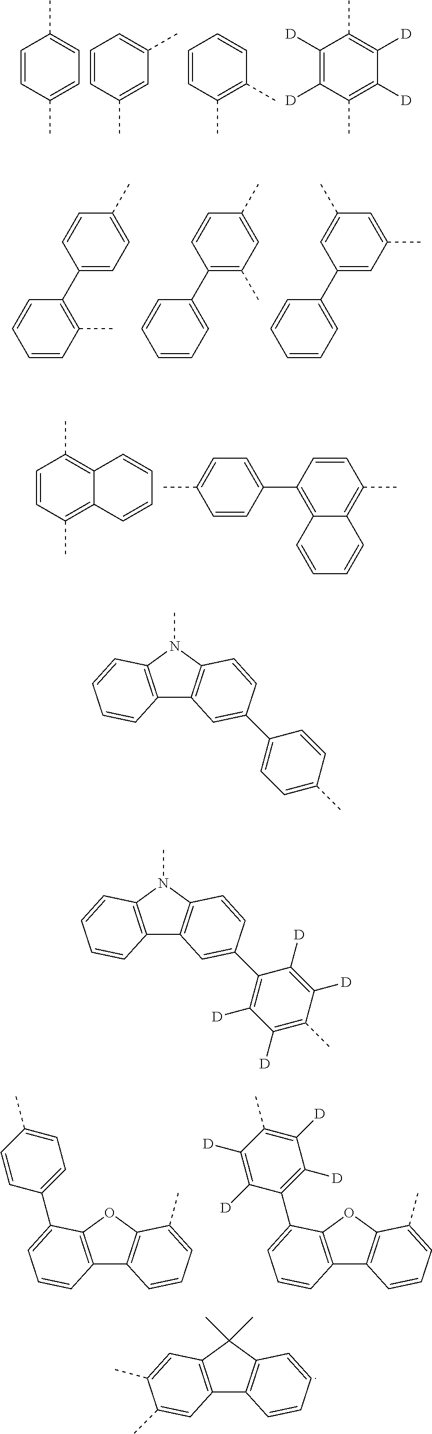

Preferably, L₇ can be a substituted or unsubstituted C₆₋₂₀ arylene,

more preferably, L₇ can be a substituted or unsubstituted phenylene, a substituted or unsubstituted biphenylylene, or a substituted or unsubstituted naphthylene, and

most preferably, L₇ can be phenylene, phenylene substituted with 4 deuteriums, biphenylylene, or naphthylene.

Preferably, the compound of Chemical Formula 2 can be of the following Chemical Formula 2-1:

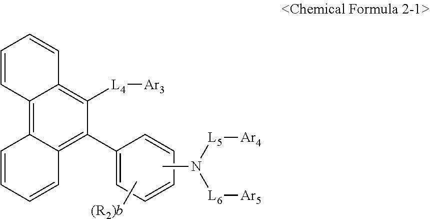

wherein in Chemical Formula 2-1:

Ar₃ to Ar₅ and L₄ to L₆ are as defined in Chemical Formula 2;

R₂ is hydrogen, deuterium, or a substituted or unsubstituted C₆₋₆₀ aryl; and

b is an integer of 0 to 4.

Preferably, R₂ can be hydrogen, deuterium, or a substituted or unsubstituted C₆₋₂₀ aryl, and

more preferably, R₂ can be hydrogen or deuterium.

Representative examples of the compound of Chemical Formula 2 are as follows:

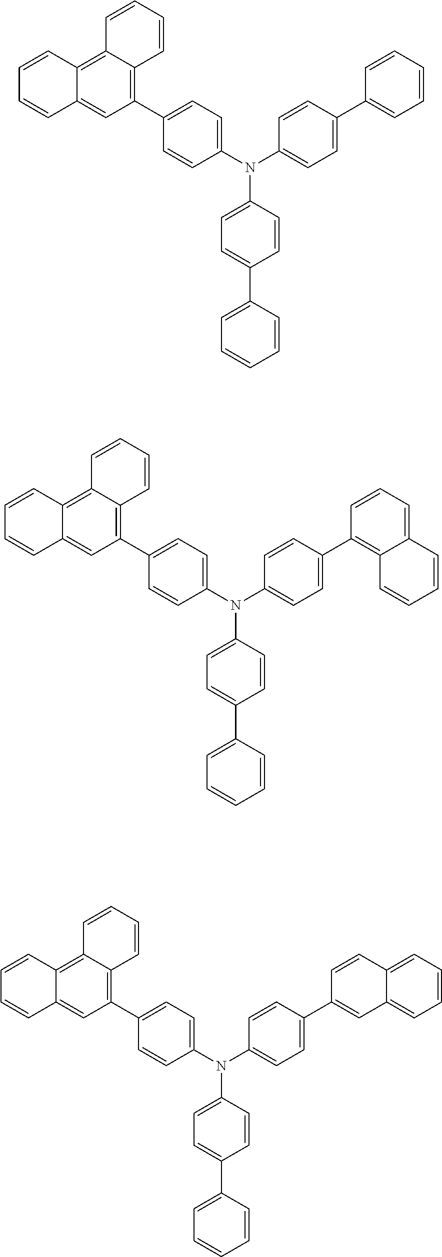 [SMILES: c1ccc(-c2ccc(N(c3ccc(-c4ccccc4)cc3)c3ccc(-c4cc5ccccc5c5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5ccccc5c4)cc3)c3ccc(-c4cc5ccccc5c5ccccc45)cc3)cc2)cc1]

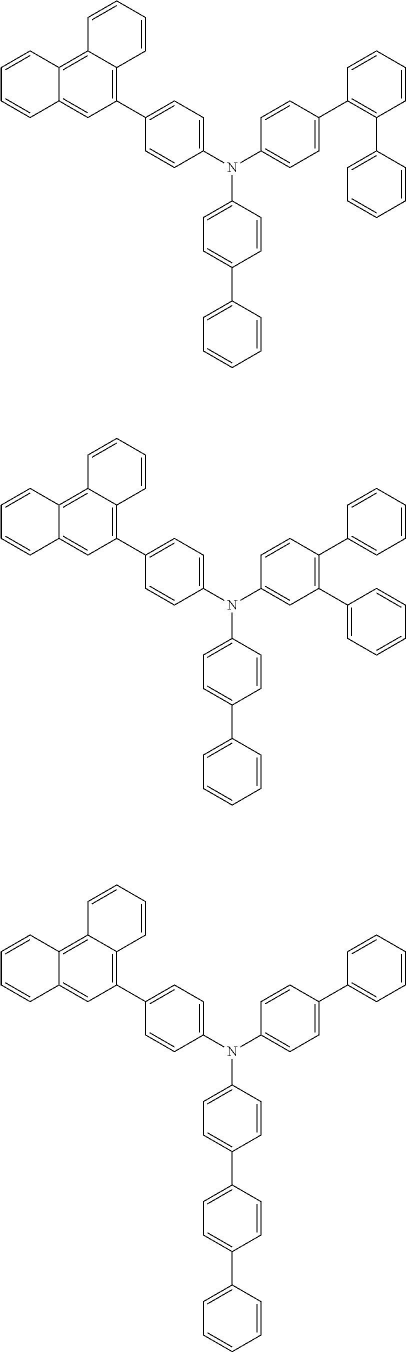 [SMILES: c1ccc(-c2ccc(N(c3ccc(-c4ccccc4-c4ccccc4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c(-c4ccccc4)c3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1]

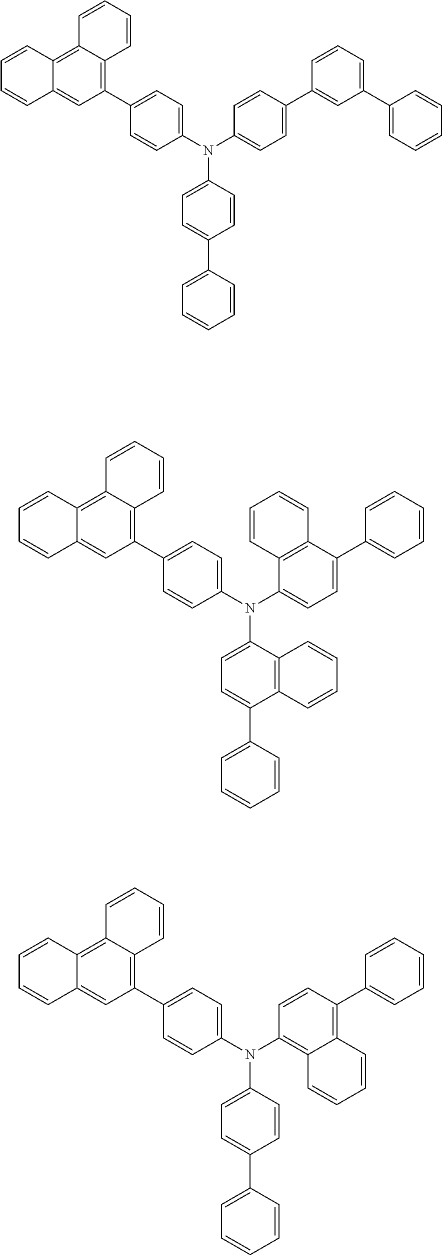 [SMILES: c1ccc(-c2ccc(N(c3ccc(-c4cccc(-c5ccccc5)c4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c4ccccc34)c3ccccc23)cc1 ; c1ccc(-c2ccc(N(c3ccc(-c4cc5ccccc5c5ccccc45)cc3)c3ccc(-c4ccccc4)c4ccccc34)cc2)cc1]

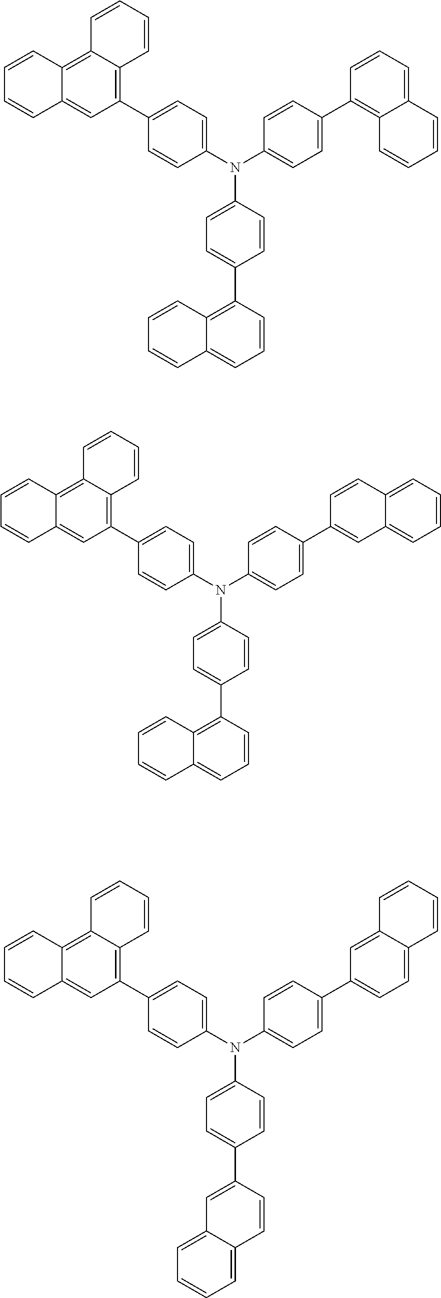 [SMILES: c1ccc2c(-c3ccc(N(c4ccc(-c5cccc6ccccc56)cc4)c4ccc(-c5cc6ccccc6c6ccccc56)cc4)cc3)cccc2c1 ; c1ccc2cc(-c3ccc(N(c4ccc(-c5cccc6ccccc56)cc4)c4ccc(-c5cc6ccccc6c6ccccc56)cc4)cc3)ccc2c1 ; c1ccc2cc(-c3ccc(N(c4ccc(-c5ccc6ccccc6c5)cc4)c4ccc(-c5cc6ccccc6c6ccccc56)cc4)cc3)ccc2c1]

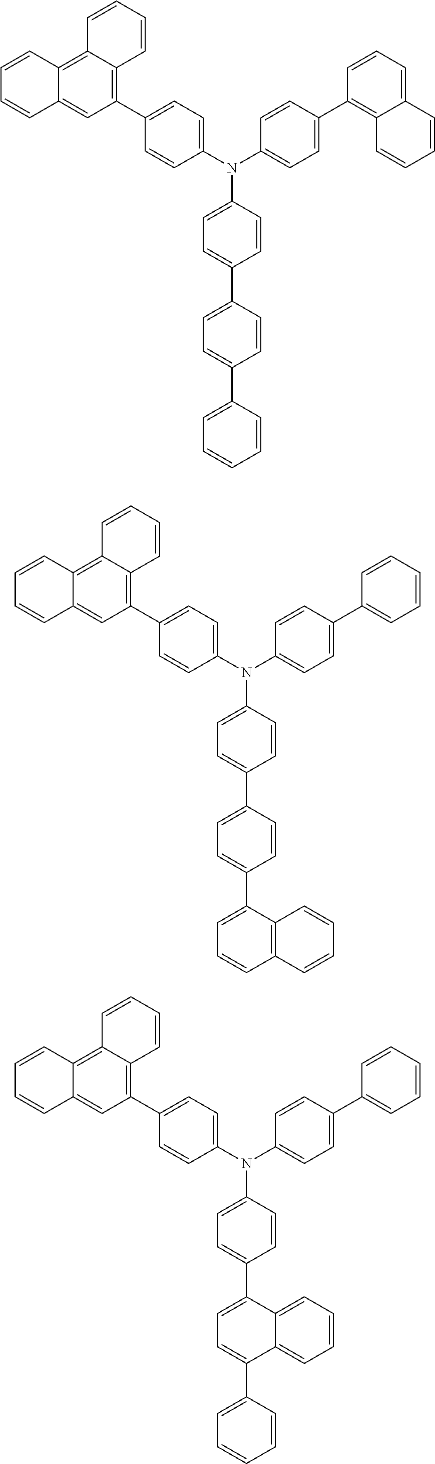 [SMILES: c1ccc(-c2ccc(-c3ccc(N(c4ccc(-c5cccc6ccccc56)cc4)c4ccc(-c5cc6ccccc6c6ccccc56)cc4)cc3)cc2)cc1 ; c1ccc(-c2ccc(N(c3ccc(-c4ccc(-c5cccc6ccccc56)cc4)cc3)c3ccc(-c4cc5ccccc5c5ccccc45)cc3)cc2)cc1 ; c1ccc(-c2ccc(N(c3ccc(-c4ccc(-c5ccccc5)c5ccccc45)cc3)c3ccc(-c4cc5ccccc5c5ccccc45)cc3)cc2)cc1]

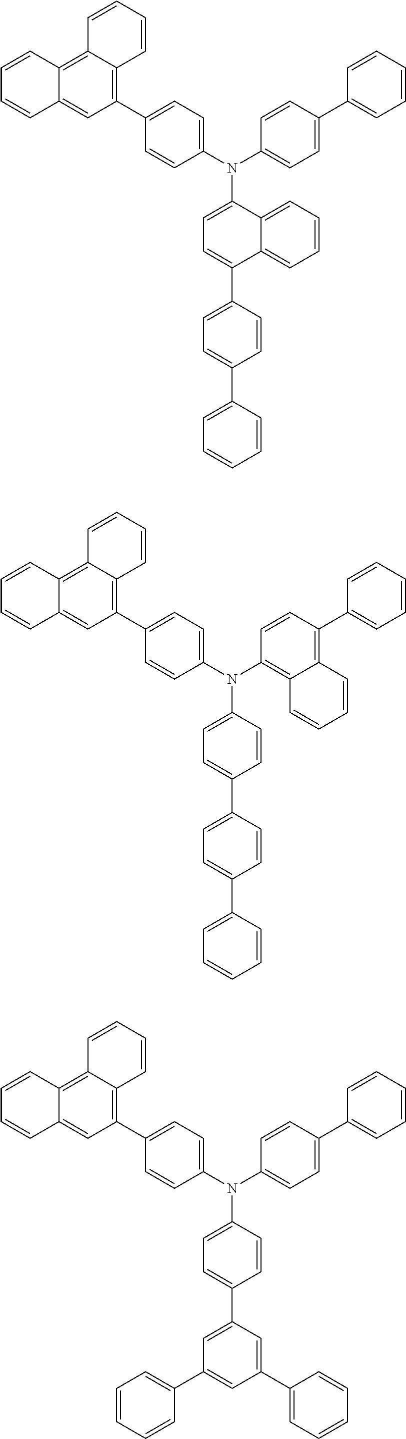 [SMILES: c1ccc(-c2ccc(-c3ccc(N(c4ccc(-c5ccccc5)cc4)c4ccc(-c5cc6ccccc6c6ccccc56)cc4)c4ccccc34)cc2)cc1 ; c1ccc(-c2ccc(-c3ccc(N(c4ccc(-c5cc6ccccc6c6ccccc56)cc4)c4ccc(-c5ccccc5)c5ccccc45)cc3)cc2)cc1 ; c1ccc(-c2ccc(N(c3ccc(-c4cc(-c5ccccc5)cc(-c5ccccc5)c4)cc3)c3ccc(-c4cc5ccccc5c5ccccc45)cc3)cc2)cc1]

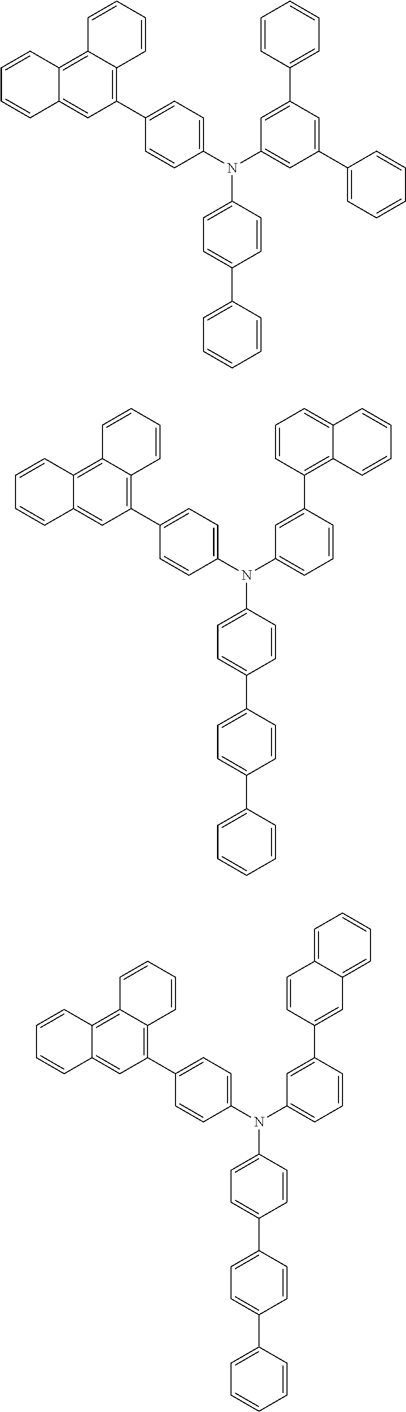 [SMILES: c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2ccc(-c3ccc(N(c4ccc(-c5cc6ccccc6c6ccccc56)cc4)c4cccc(-c5cccc6ccccc56)c4)cc3)cc2)cc1 ; c1ccc(-c2ccc(-c3ccc(N(c4ccc(-c5cc6ccccc6c6ccccc56)cc4)c4cccc(-c5ccc6ccccc6c5)c4)cc3)cc2)cc1]

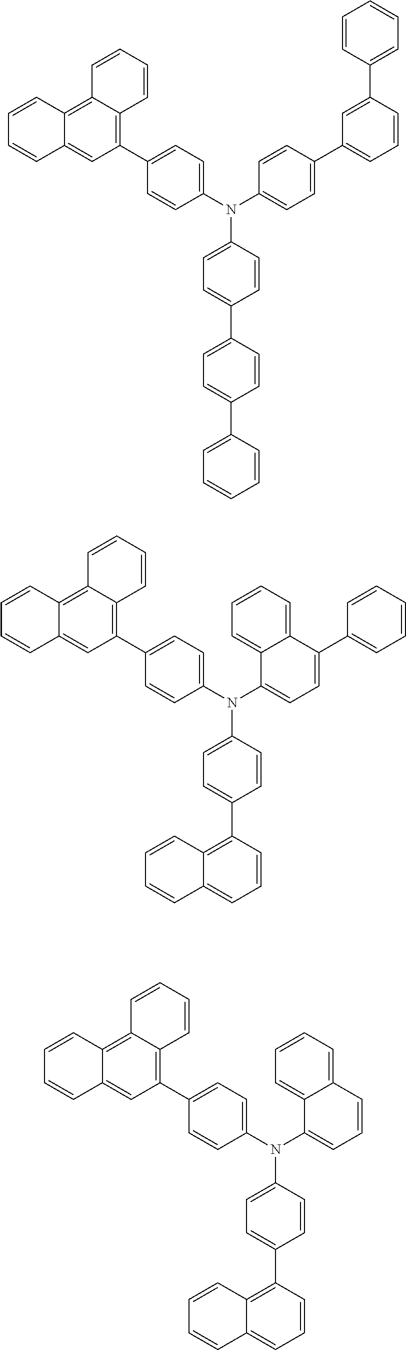 [SMILES: c1ccc(-c2ccc(-c3ccc(N(c4ccc(-c5cccc(-c6ccccc6)c5)cc4)c4ccc(-c5cc6ccccc6c6ccccc56)cc4)cc3)cc2)cc1 ; c1ccc(-c2ccc(N(c3ccc(-c4cccc5ccccc45)cc3)c3ccc(-c4cc5ccccc5c5ccccc45)cc3)c3ccccc23)cc1 ; c1ccc2c(-c3ccc(N(c4ccc(-c5cc6ccccc6c6ccccc56)cc4)c4cccc5ccccc45)cc3)cccc2c1]

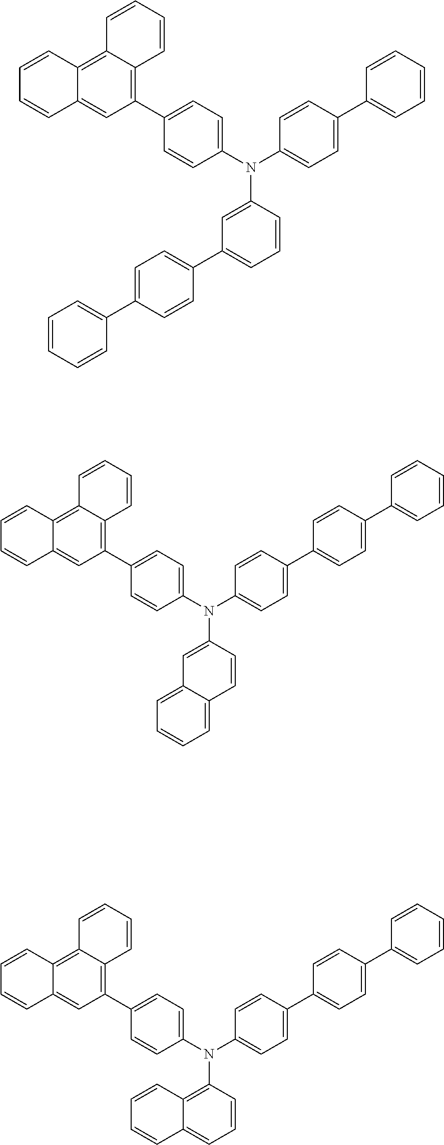 [SMILES: c1ccc(-c2ccc(-c3cccc(N(c4ccc(-c5ccccc5)cc4)c4ccc(-c5cc6ccccc6c6ccccc56)cc4)c3)cc2)cc1 ; c1ccc(-c2ccc(-c3ccc(N(c4ccc(-c5cc6ccccc6c6ccccc56)cc4)c4ccc5ccccc5c4)cc3)cc2)cc1 ; c1ccc(-c2ccc(-c3ccc(N(c4ccc(-c5cc6ccccc6c6ccccc56)cc4)c4cccc5ccccc45)cc3)cc2)cc1]

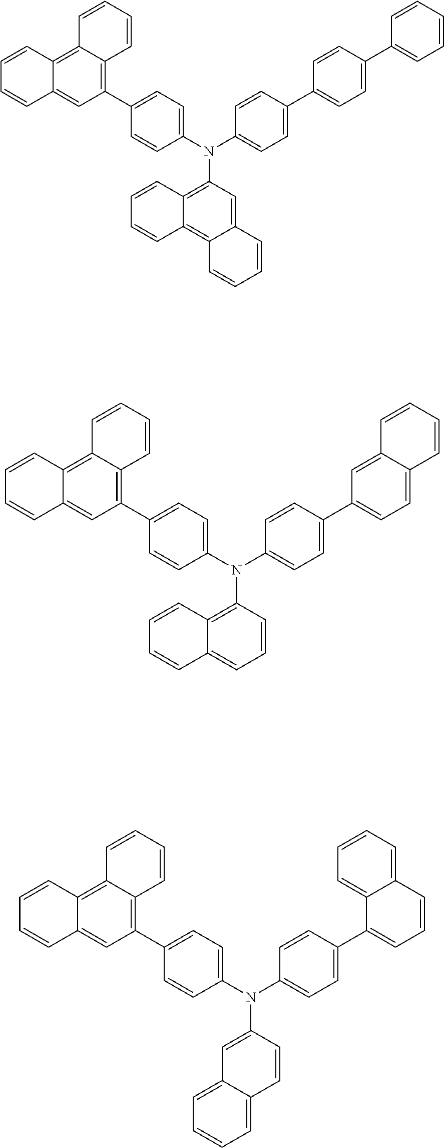 [SMILES: c1ccc(-c2ccc(-c3ccc(N(c4ccc(-c5cc6ccccc6c6ccccc56)cc4)c4cc5ccccc5c5ccccc45)cc3)cc2)cc1 ; c1ccc2cc(-c3ccc(N(c4ccc(-c5cc6ccccc6c6ccccc56)cc4)c4cccc5ccccc45)cc3)ccc2c1 ; c1ccc2cc(N(c3ccc(-c4cccc5ccccc45)cc3)c3ccc(-c4cc5ccccc5c5ccccc45)cc3)ccc2c1]

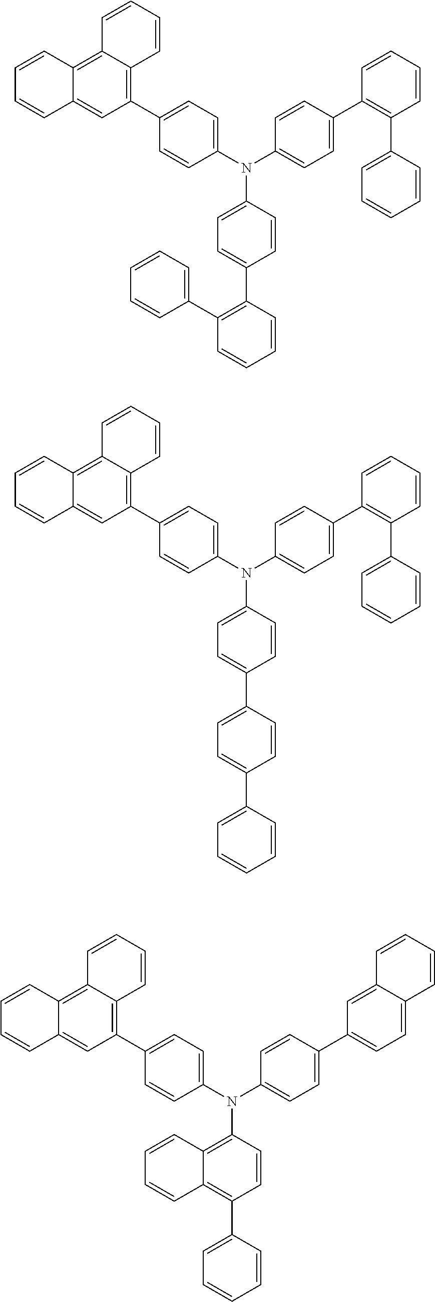 [SMILES: c1ccc(-c2ccccc2-c2ccc(N(c3ccc(-c4ccccc4-c4ccccc4)cc3)c3ccc(-c4cc5ccccc5c5ccccc45)cc3)cc2)cc1 ; c1ccc(-c2ccc(-c3ccc(N(c4ccc(-c5ccccc5-c5ccccc5)cc4)c4ccc(-c5cc6ccccc6c6ccccc56)cc4)cc3)cc2)cc1 ; c1ccc(-c2ccc(N(c3ccc(-c4ccc5ccccc5c4)cc3)c3ccc(-c4cc5ccccc5c5ccccc45)cc3)c3ccccc23)cc1]

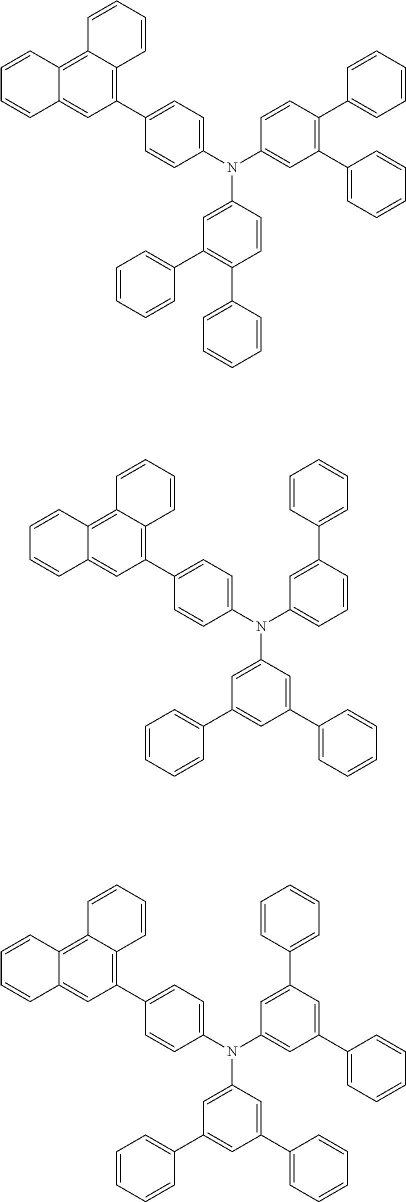 [SMILES: c1ccc(-c2ccc(N(c3ccc(-c4cc5ccccc5c5ccccc45)cc3)c3ccc(-c4ccccc4)c(-c4ccccc4)c3)cc2-c2ccccc2)cc1 ; c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2cc(-c3ccccc3)cc(N(c3ccc(-c4cc5ccccc5c5ccccc45)cc3)c3cc(-c4ccccc4)cc(-c4ccccc4)c3)c2)cc1]

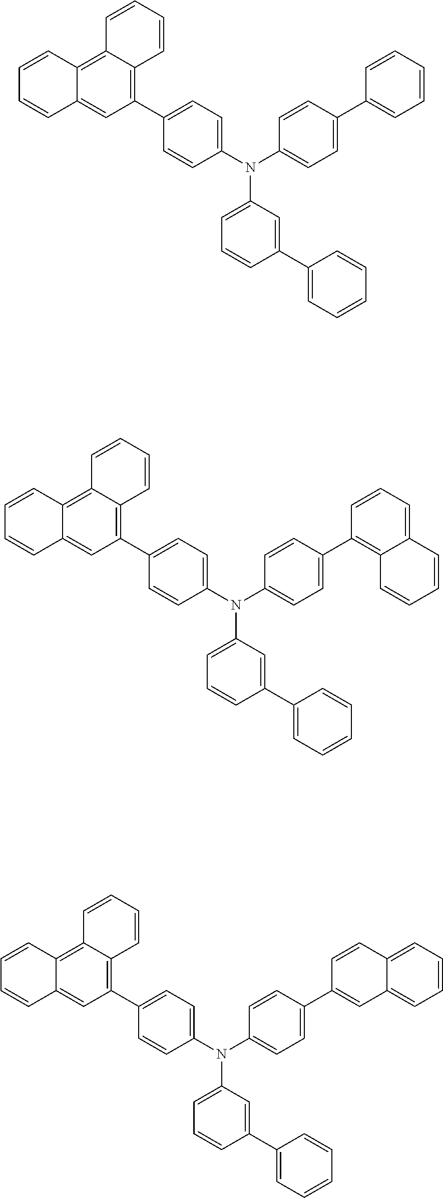 [SMILES: c1ccc(-c2ccc(N(c3ccc(-c4cc5ccccc5c5ccccc45)cc3)c3cccc(-c4ccccc4)c3)cc2)cc1 ; c1ccc(-c2cccc(N(c3ccc(-c4cccc5ccccc45)cc3)c3ccc(-c4cc5ccccc5c5ccccc45)cc3)c2)cc1 ; c1ccc(-c2cccc(N(c3ccc(-c4ccc5ccccc5c4)cc3)c3ccc(-c4cc5ccccc5c5ccccc45)cc3)c2)cc1]

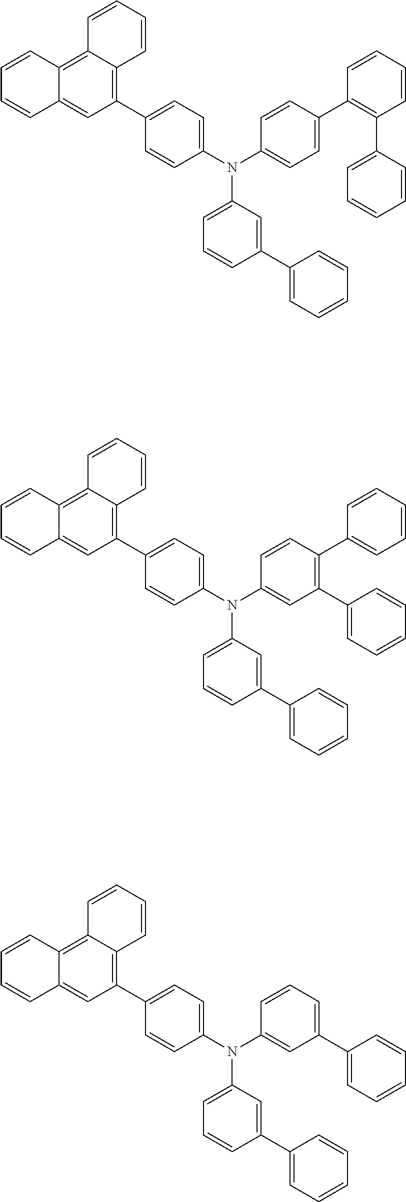 [SMILES: c1ccc(-c2cccc(N(c3ccc(-c4ccccc4-c4ccccc4)cc3)c3ccc(-c4cc5ccccc5c5ccccc45)cc3)c2)cc1 ; c1ccc(-c2cccc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cccc(-c4ccccc4)c3)c2)cc1]

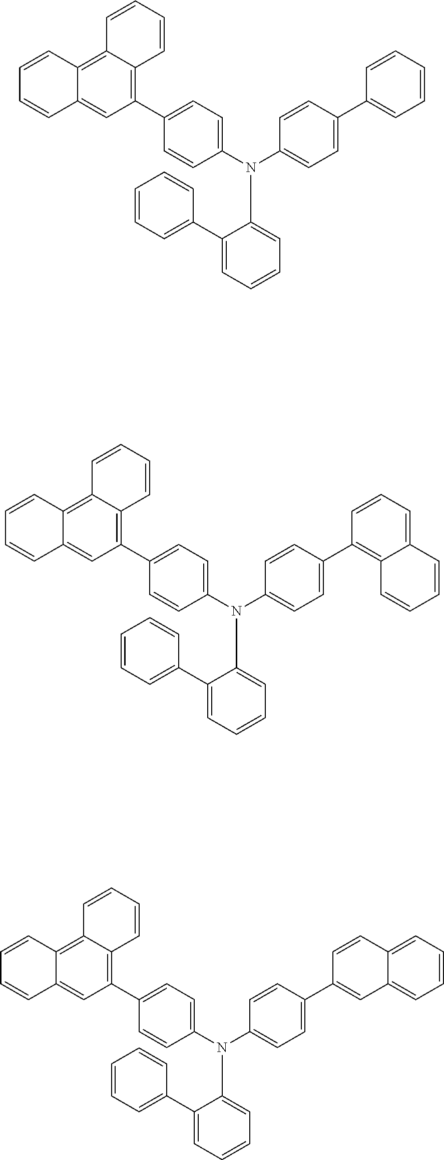 [SMILES: c1ccc(-c2ccc(N(c3ccc(-c4cc5ccccc5c5ccccc45)cc3)c3ccccc3-c3ccccc3)cc2)cc1 ; c1ccc(-c2ccccc2N(c2ccc(-c3cccc4ccccc34)cc2)c2ccc(-c3cc4ccccc4c4ccccc34)cc2)cc1 ; c1ccc(-c2ccccc2N(c2ccc(-c3ccc4ccccc4c3)cc2)c2ccc(-c3cc4ccccc4c4ccccc34)cc2)cc1]

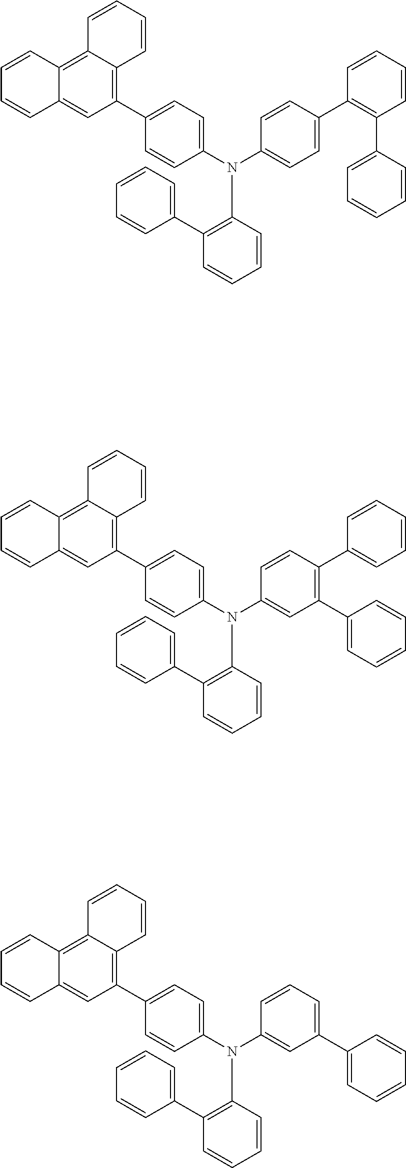 [SMILES: c1ccc(-c2ccccc2-c2ccc(N(c3ccc(-c4cc5ccccc5c5ccccc45)cc3)c3ccccc3-c3ccccc3)cc2)cc1 ; c1ccc(-c2ccc(N(c3ccc(-c4cc5ccccc5c5ccccc45)cc3)c3ccccc3-c3ccccc3)cc2-c2ccccc2)cc1 ; c1ccc(-c2cccc(N(c3ccc(-c4cc5ccccc5c5ccccc45)cc3)c3ccccc3-c3ccccc3)c2)cc1]

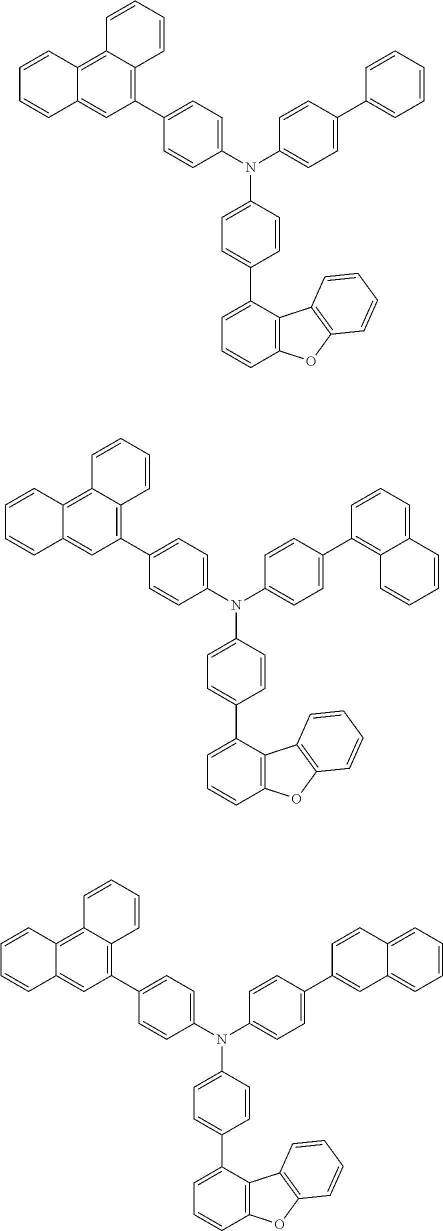 [SMILES: c1ccc(-c2ccc(N(c3ccc(-c4cc5ccccc5c5ccccc45)cc3)c3ccc(-c4cccc5oc6ccccc6c45)cc3)cc2)cc1 ; c1ccc2c(-c3ccc(N(c4ccc(-c5cc6ccccc6c6ccccc56)cc4)c4ccc(-c5cccc6oc7ccccc7c56)cc4)cc3)cccc2c1 ; c1ccc2cc(-c3ccc(N(c4ccc(-c5cc6ccccc6c6ccccc56)cc4)c4ccc(-c5cccc6oc7ccccc7c56)cc4)cc3)ccc2c1]

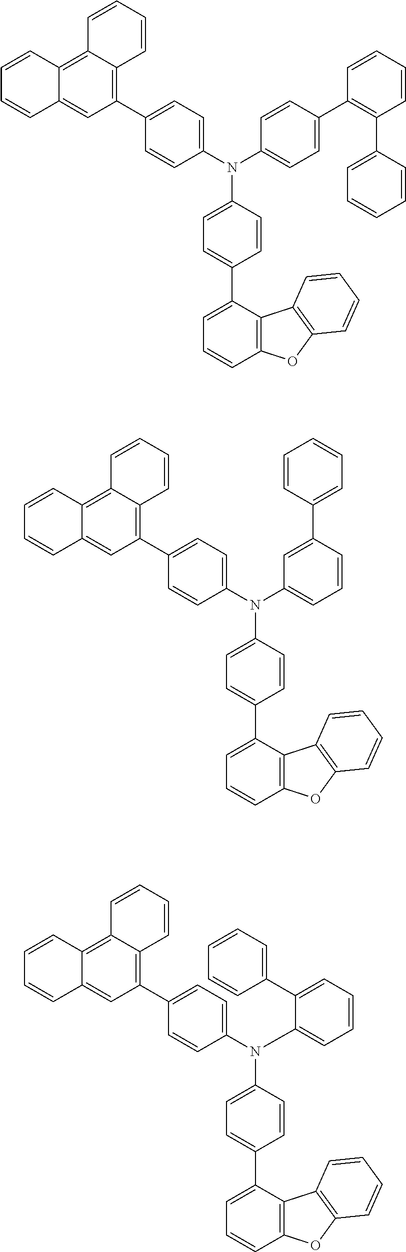 [SMILES: c1ccc(-c2ccccc2-c2ccc(N(c3ccc(-c4cc5ccccc5c5ccccc45)cc3)c3ccc(-c4cccc5oc6ccccc6c45)cc3)cc2)cc1 ; c1ccc(-c2cccc(N(c3ccc(-c4cc5ccccc5c5ccccc45)cc3)c3ccc(-c4cccc5oc6ccccc6c45)cc3)c2)cc1 ; c1ccc(-c2ccccc2N(c2ccc(-c3cc4ccccc4c4ccccc34)cc2)c2ccc(-c3cccc4oc5ccccc5c34)cc2)cc1]

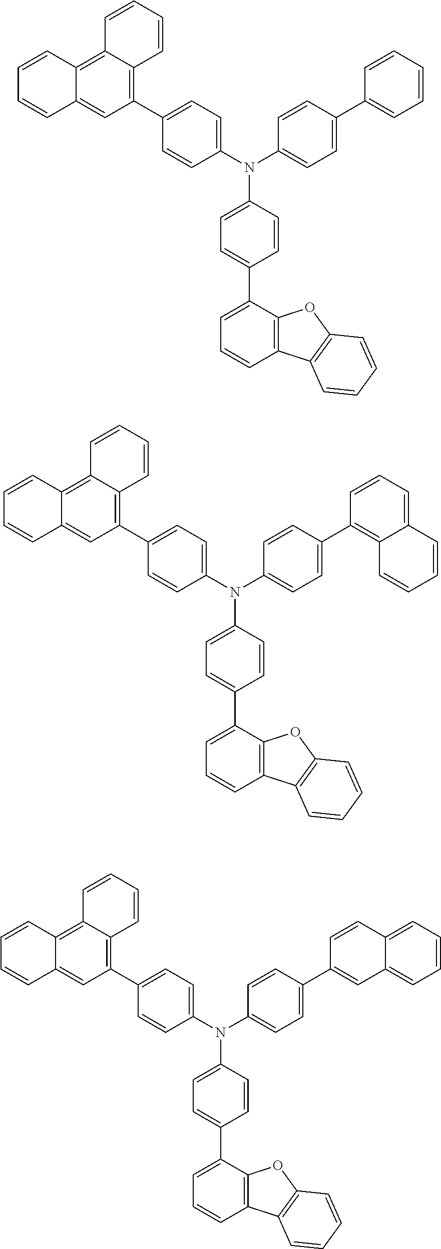 [SMILES: c1ccc(-c2ccc(N(c3ccc(-c4cc5ccccc5c5ccccc45)cc3)c3ccc(-c4cccc5c4oc4ccccc45)cc3)cc2)cc1 ; c1ccc2c(-c3ccc(N(c4ccc(-c5cc6ccccc6c6ccccc56)cc4)c4ccc(-c5cccc6c5oc5ccccc56)cc4)cc3)cccc2c1 ; c1ccc2cc(-c3ccc(N(c4ccc(-c5cc6ccccc6c6ccccc56)cc4)c4ccc(-c5cccc6c5oc5ccccc56)cc4)cc3)ccc2c1]

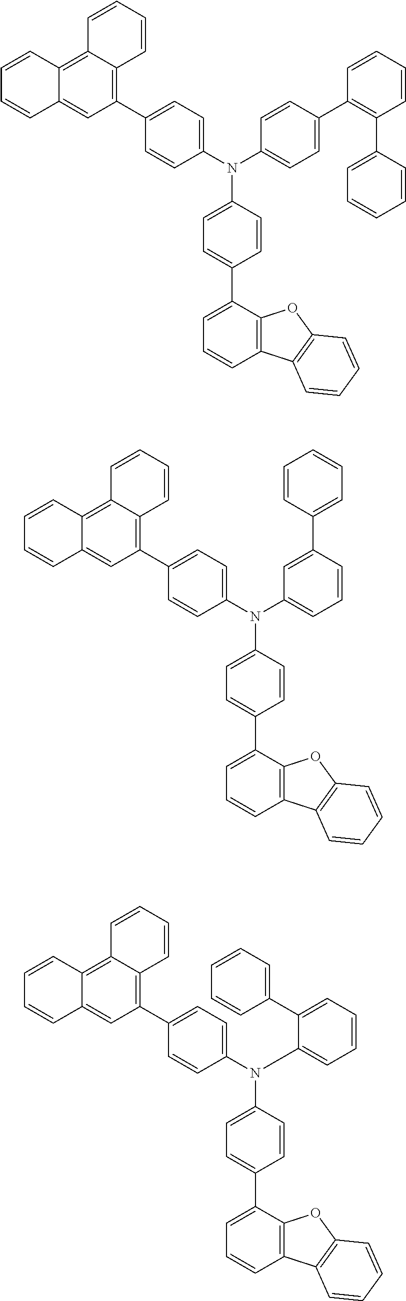 [SMILES: c1ccc(-c2ccccc2-c2ccc(N(c3ccc(-c4cc5ccccc5c5ccccc45)cc3)c3ccc(-c4cccc5c4oc4ccccc45)cc3)cc2)cc1 ; c1ccc(-c2cccc(N(c3ccc(-c4cc5ccccc5c5ccccc45)cc3)c3ccc(-c4cccc5c4oc4ccccc45)cc3)c2)cc1 ; c1ccc(-c2ccccc2N(c2ccc(-c3cc4ccccc4c4ccccc34)cc2)c2ccc(-c3cccc4c3oc3ccccc34)cc2)cc1]

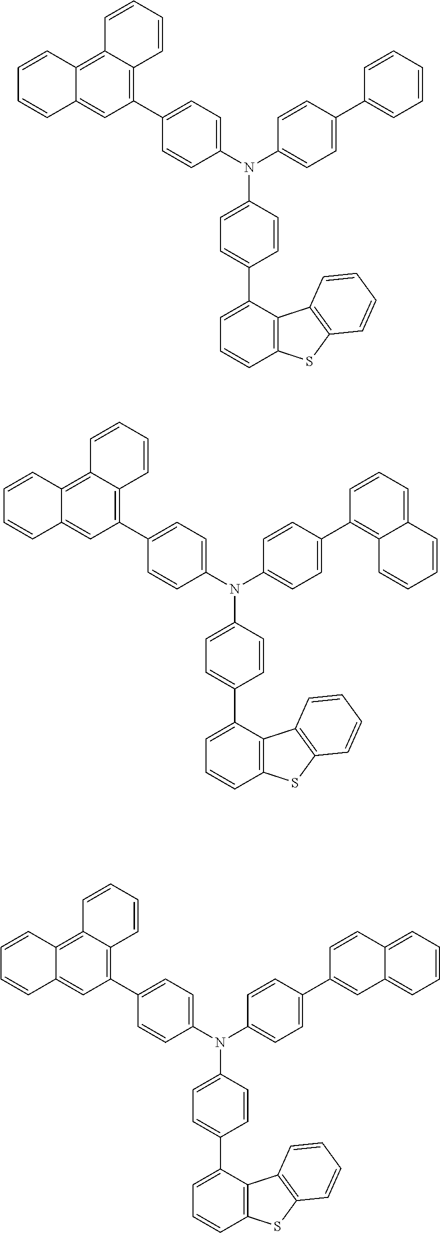 [SMILES: c1ccc(-c2ccc(N(c3ccc(-c4cc5ccccc5c5ccccc45)cc3)c3ccc(-c4cccc5sc6ccccc6c45)cc3)cc2)cc1 ; c1ccc2c(-c3ccc(N(c4ccc(-c5cc6ccccc6c6ccccc56)cc4)c4ccc(-c5cccc6sc7ccccc7c56)cc4)cc3)cccc2c1 ; c1ccc2cc(-c3ccc(N(c4ccc(-c5cc6ccccc6c6ccccc56)cc4)c4ccc(-c5cccc6sc7ccccc7c56)cc4)cc3)ccc2c1]

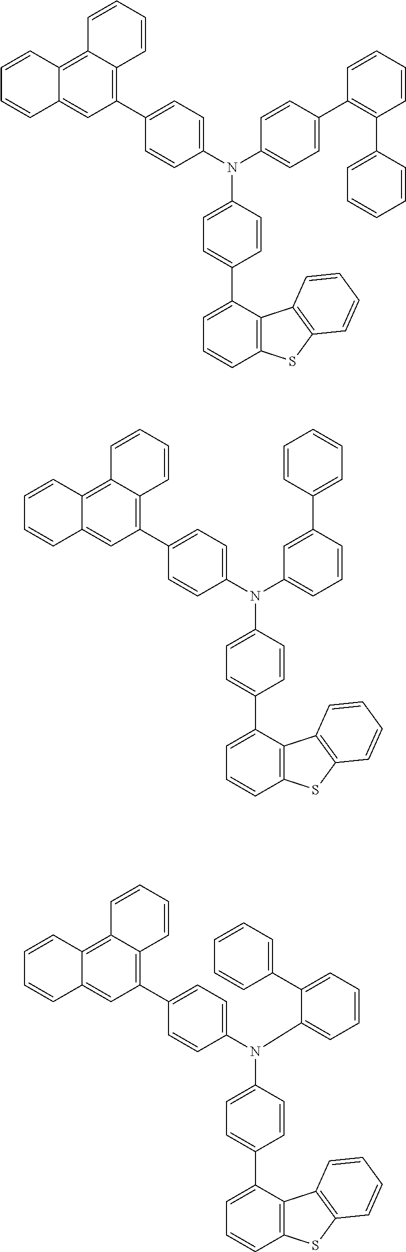 [SMILES: c1ccc(-c2ccccc2-c2ccc(N(c3ccc(-c4cc5ccccc5c5ccccc45)cc3)c3ccc(-c4cccc5sc6ccccc6c45)cc3)cc2)cc1 ; c1ccc(-c2cccc(N(c3ccc(-c4cc5ccccc5c5ccccc45)cc3)c3ccc(-c4cccc5sc6ccccc6c45)cc3)c2)cc1 ; c1ccc(-c2ccccc2N(c2ccc(-c3cc4ccccc4c4ccccc34)cc2)c2ccc(-c3cccc4sc5ccccc5c34)cc2)cc1]

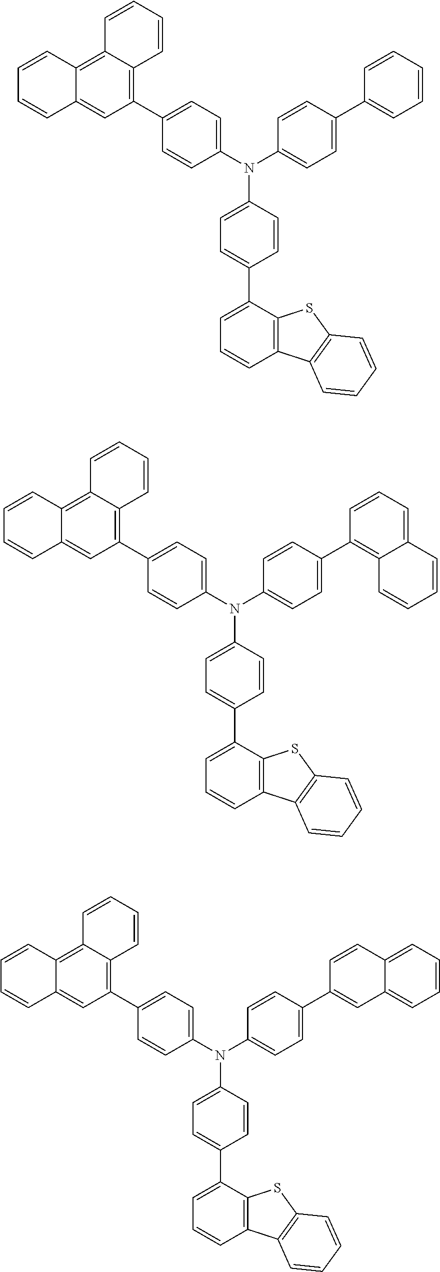 [SMILES: c1ccc(-c2ccc(N(c3ccc(-c4cc5ccccc5c5ccccc45)cc3)c3ccc(-c4cccc5c4sc4ccccc45)cc3)cc2)cc1 ; c1ccc2c(-c3ccc(N(c4ccc(-c5cc6ccccc6c6ccccc56)cc4)c4ccc(-c5cccc6c5sc5ccccc56)cc4)cc3)cccc2c1 ; c1ccc2cc(-c3ccc(N(c4ccc(-c5cc6ccccc6c6ccccc56)cc4)c4ccc(-c5cccc6c5sc5ccccc56)cc4)cc3)ccc2c1]

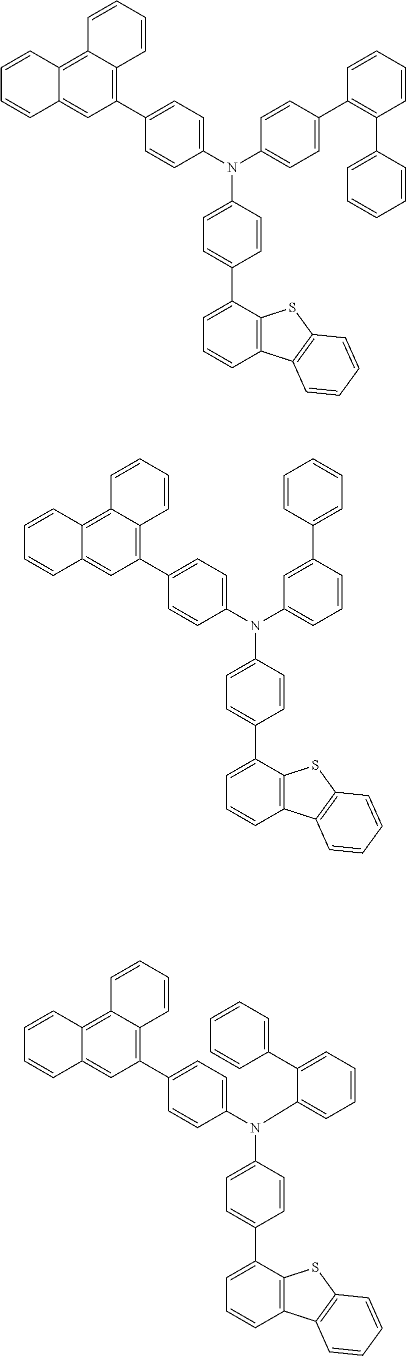 [SMILES: c1ccc(-c2ccccc2-c2ccc(N(c3ccc(-c4cc5ccccc5c5ccccc45)cc3)c3ccc(-c4cccc5c4sc4ccccc45)cc3)cc2)cc1 ; c1ccc(-c2cccc(N(c3ccc(-c4cc5ccccc5c5ccccc45)cc3)c3ccc(-c4cccc5c4sc4ccccc45)cc3)c2)cc1 ; c1ccc(-c2ccccc2N(c2ccc(-c3cc4ccccc4c4ccccc34)cc2)c2ccc(-c3cccc4c3sc3ccccc34)cc2)cc1]

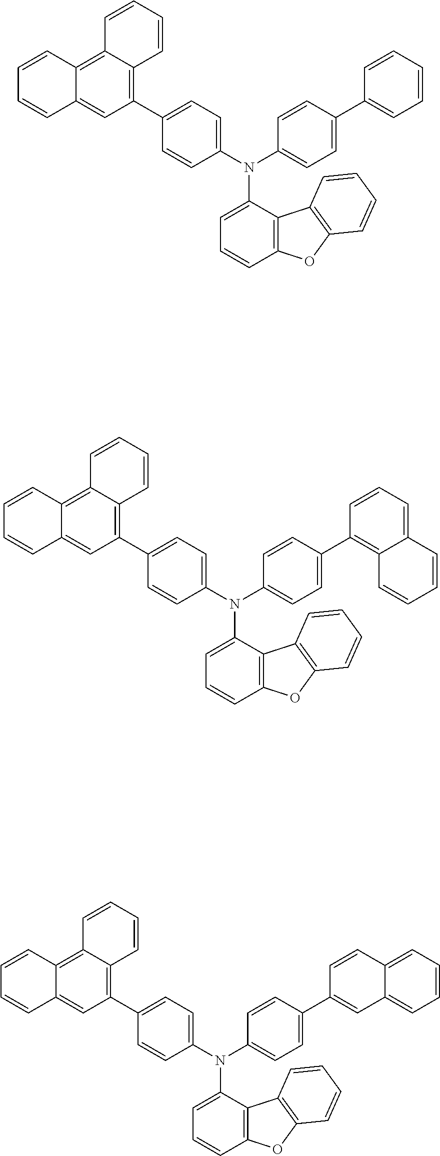 [SMILES: c1ccc(-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)cc3)cccc2c1 ; c1ccc2cc(-c3ccc(N(c4ccc(-c5cc6ccccc6c6ccccc56)cc4)c4cccc5oc6ccccc6c45)cc3)ccc2c1]

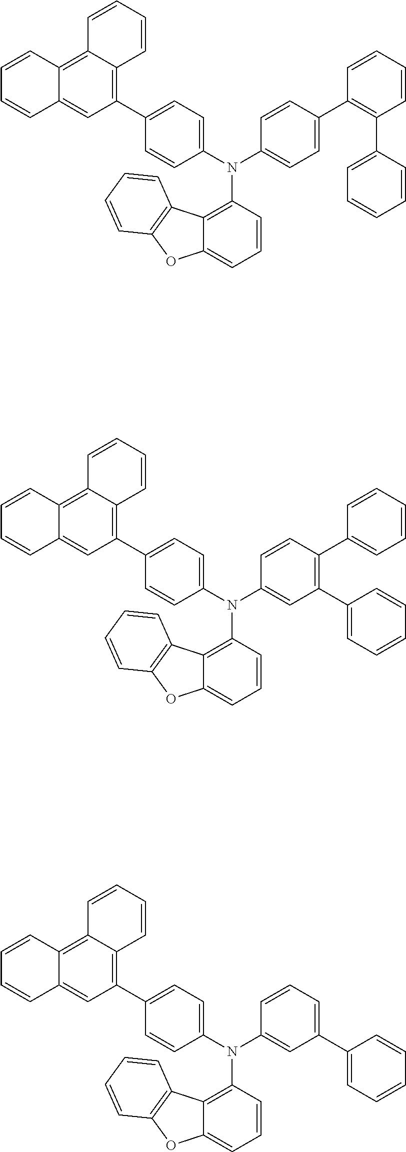 [SMILES: c1ccc(-c2ccccc2-c2ccc(N(c3ccc(-c4cc5ccccc5c5ccccc45)cc3)c3cccc4oc5ccccc5c34)cc2)cc1 ; c1ccc(-c2ccc(N(c3ccc(-c4cc5ccccc5c5ccccc45)cc3)c3cccc4oc5ccccc5c34)cc2-c2ccccc2)cc1 ; c1ccc(-c2cccc(N(c3ccc(-c4cc5ccccc5c5ccccc45)cc3)c3cccc4oc5ccccc5c34)c2)cc1]

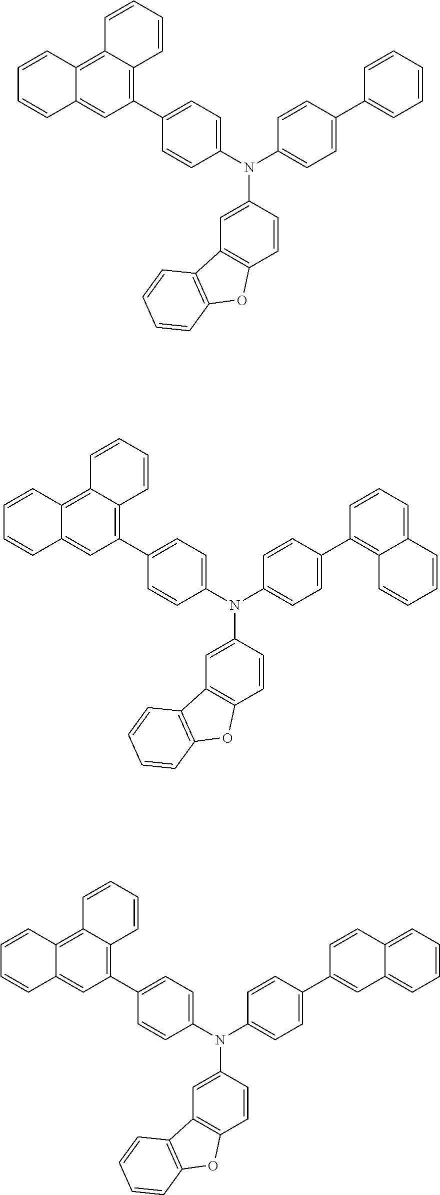 [SMILES: c1ccc(-c2ccc(N(c3ccc(-c4cc5ccccc5c5ccccc45)cc3)c3ccc4oc5ccccc5c4c3)cc2)cc1 ; c1ccc2c(-c3ccc(N(c4ccc(-c5cc6ccccc6c6ccccc56)cc4)c4ccc5oc6ccccc6c5c4)cc3)cccc2c1 ; c1ccc2cc(-c3ccc(N(c4ccc(-c5cc6ccccc6c6ccccc56)cc4)c4ccc5oc6ccccc6c5c4)cc3)ccc2c1]

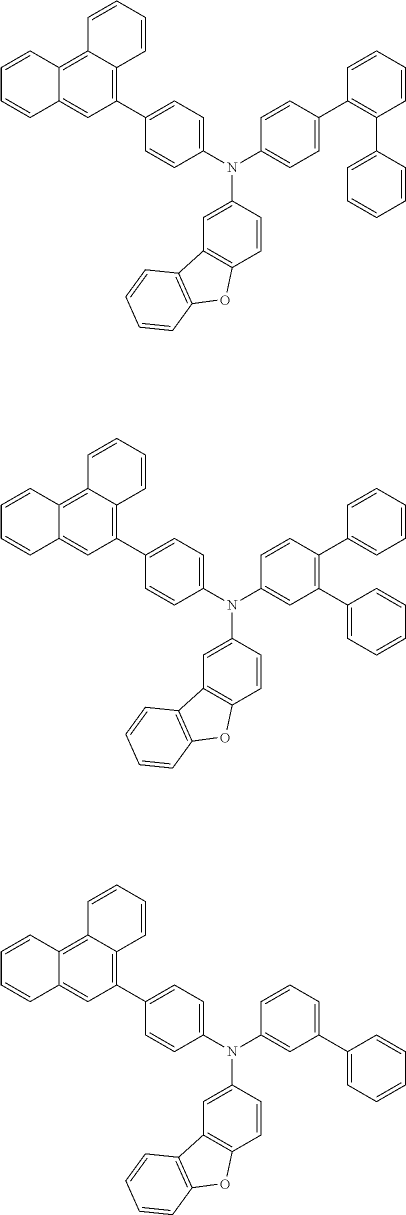 [SMILES: c1ccc(-c2ccccc2-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4oc5ccccc5c4c3)cc2-c2ccccc2)cc1 ; c1ccc(-c2cccc(N(c3ccc(-c4cc5ccccc5c5ccccc45)cc3)c3ccc4oc5ccccc5c4c3)c2)cc1]

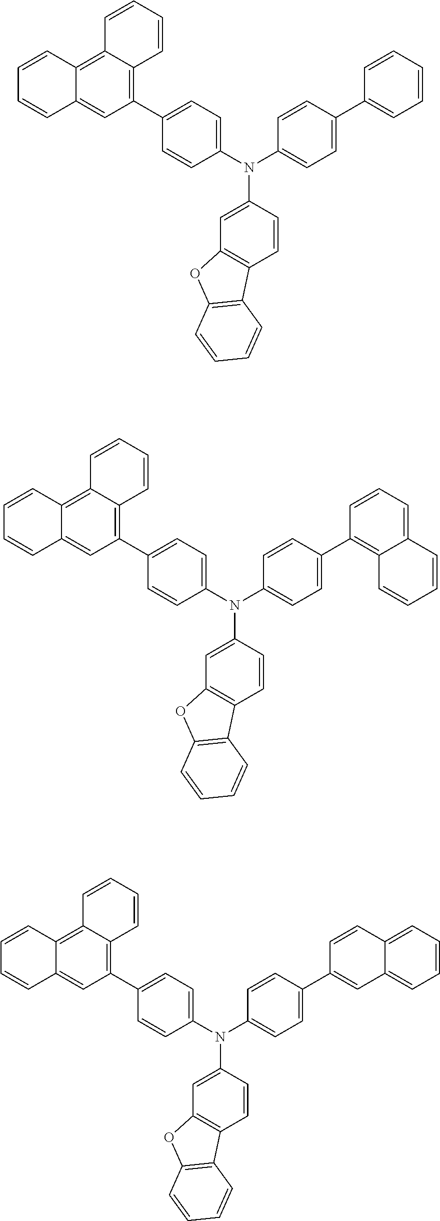 [SMILES: c1ccc(-c2ccc(N(c3ccc(-c4cc5ccccc5c5ccccc45)cc3)c3ccc4c(c3)oc3ccccc34)cc2)cc1 ; c1ccc2c(-c3ccc(N(c4ccc(-c5cc6ccccc6c6ccccc56)cc4)c4ccc5c(c4)oc4ccccc45)cc3)cccc2c1 ; c1ccc2cc(-c3ccc(N(c4ccc(-c5cc6ccccc6c6ccccc56)cc4)c4ccc5c(c4)oc4ccccc45)cc3)ccc2c1]

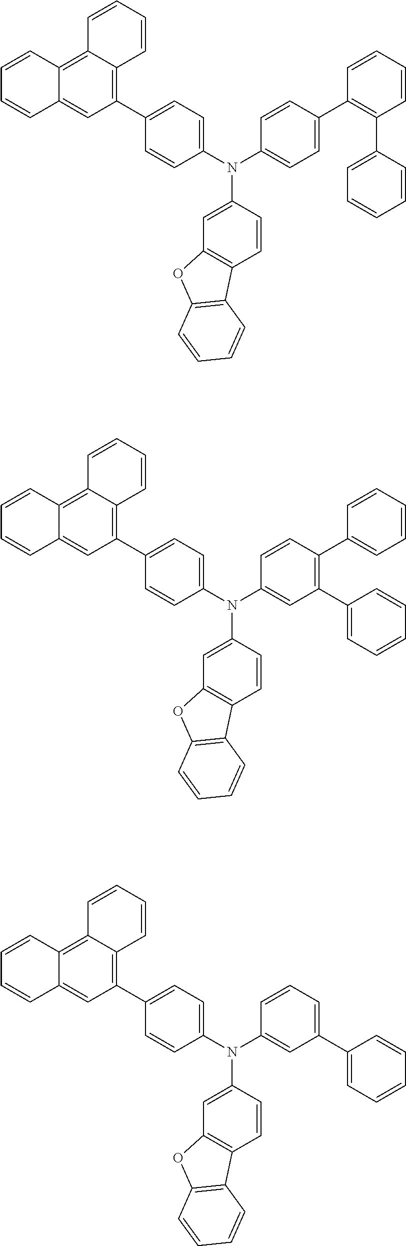 [SMILES: c1ccc(-c2ccccc2-c2ccc(N(c3ccc(-c4cc5ccccc5c5ccccc45)cc3)c3ccc4c(c3)oc3ccccc34)cc2)cc1 ; c1ccc(-c2ccc(N(c3ccc(-c4cc5ccccc5c5ccccc45)cc3)c3ccc4c(c3)oc3ccccc34)cc2-c2ccccc2)cc1 ; c1ccc(-c2cccc(N(c3ccc(-c4cc5ccccc5c5ccccc45)cc3)c3ccc4c(c3)oc3ccccc34)c2)cc1]

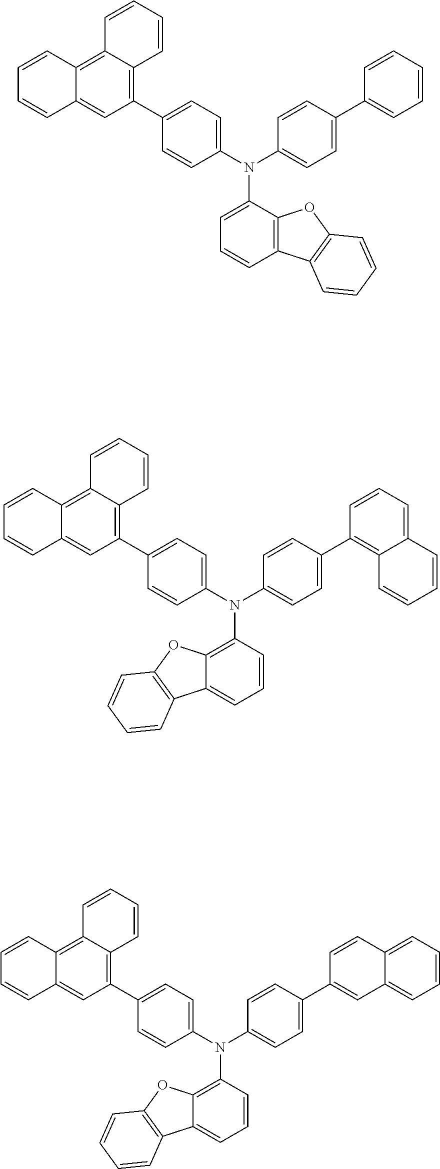 [SMILES: c1ccc(-c2ccc(N(c3ccc(-c4cc5ccccc5c5ccccc45)cc3)c3cccc4c3oc3ccccc34)cc2)cc1 ; c1ccc2c(-c3ccc(N(c4ccc(-c5cc6ccccc6c6ccccc56)cc4)c4cccc5c4oc4ccccc45)cc3)cccc2c1 ; c1ccc2cc(-c3ccc(N(c4ccc(-c5cc6ccccc6c6ccccc56)cc4)c4cccc5c4oc4ccccc45)cc3)ccc2c1]

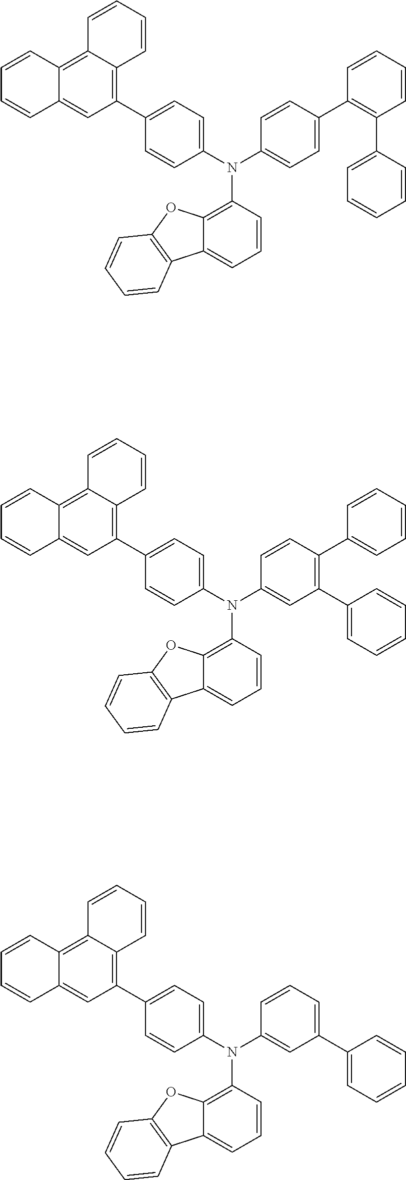 [SMILES: c1ccc(-c2ccccc2-c2ccc(N(c3ccc(-c4cc5ccccc5c5ccccc45)cc3)c3cccc4c3oc3ccccc34)cc2)cc1 ; c1ccc(-c2ccc(N(c3ccc(-c4cc5ccccc5c5ccccc45)cc3)c3cccc4c3oc3ccccc34)cc2-c2ccccc2)cc1 ; c1ccc(-c2cccc(N(c3ccc(-c4cc5ccccc5c5ccccc45)cc3)c3cccc4c3oc3ccccc34)c2)cc1]

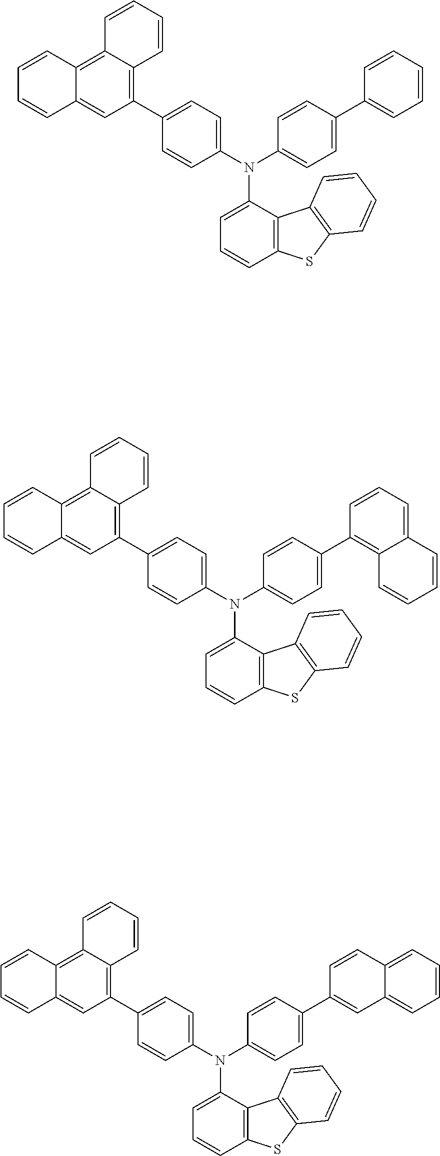 [SMILES: c1ccc(-c2ccc(N(c3ccc(-c4cc5ccccc5c5ccccc45)cc3)c3cccc4sc5ccccc5c34)cc2)cc1 ; c1ccc2c(-c3ccc(N(c4ccc(-c5cc6ccccc6c6ccccc56)cc4)c4cccc5sc6ccccc6c45)cc3)cccc2c1 ; c1ccc2cc(-c3ccc(N(c4ccc(-c5cc6ccccc6c6ccccc56)cc4)c4cccc5sc6ccccc6c45)cc3)ccc2c1]

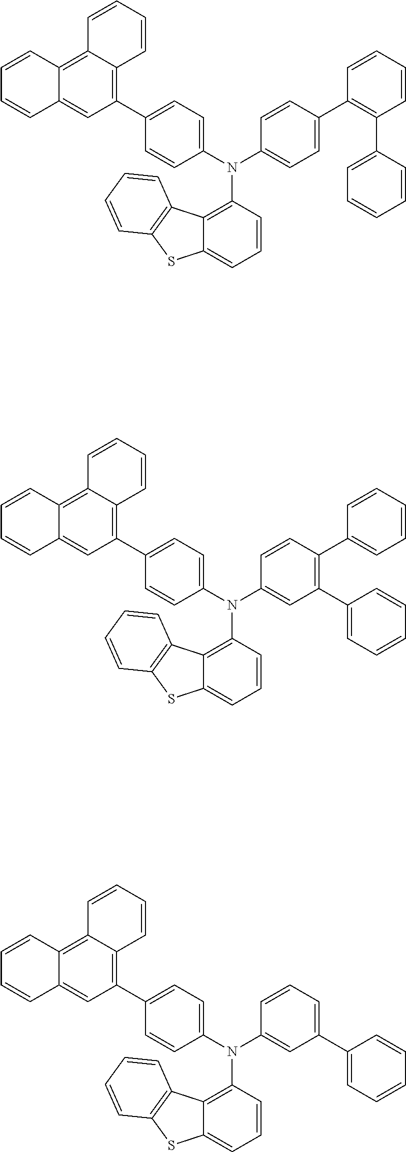 [SMILES: c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)cc2-c2ccccc2)cc1 ; c1ccc(-c2cccc(N(c3ccc(-c4cc5ccccc5c5ccccc45)cc3)c3cccc4sc5ccccc5c34)c2)cc1]

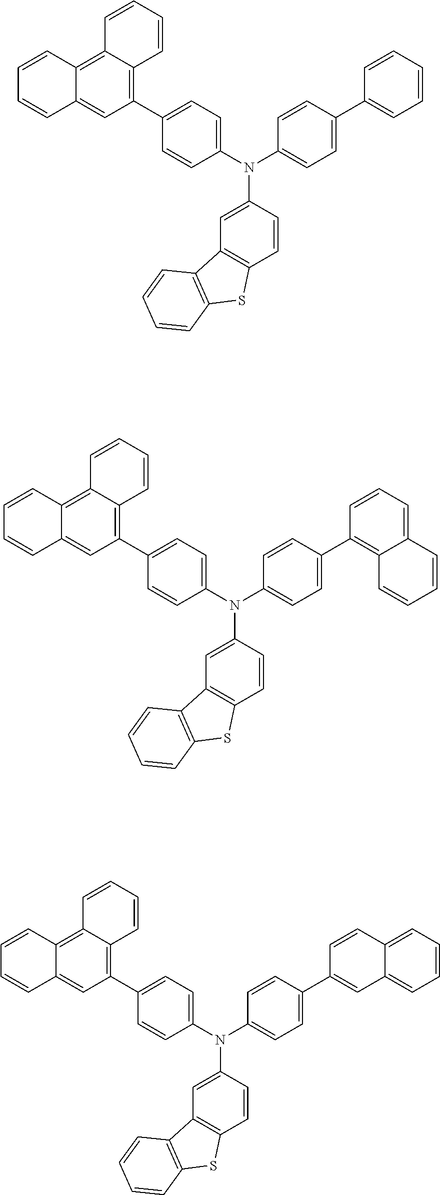 [SMILES: c1ccc(-c2ccc(N(c3ccc(-c4cc5ccccc5c5ccccc45)cc3)c3ccc4sc5ccccc5c4c3)cc2)cc1 ; c1ccc2c(-c3ccc(N(c4ccc(-c5cc6ccccc6c6ccccc56)cc4)c4ccc5sc6ccccc6c5c4)cc3)cccc2c1 ; c1ccc2cc(-c3ccc(N(c4ccc(-c5cc6ccccc6c6ccccc56)cc4)c4ccc5sc6ccccc6c5c4)cc3)ccc2c1]

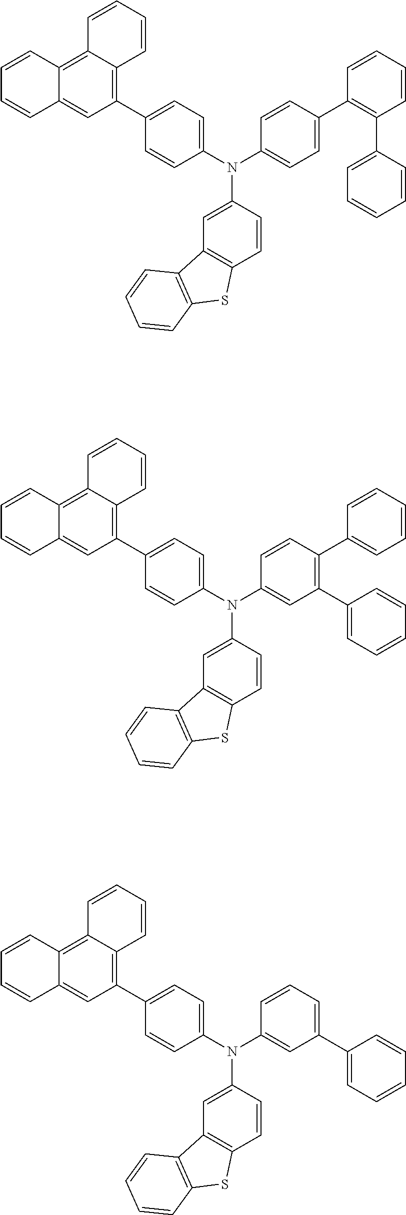 [SMILES: c1ccc(-c2ccccc2-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4sc5ccccc5c4c3)cc2-c2ccccc2)cc1 ; c1ccc(-c2cccc(N(c3ccc(-c4cc5ccccc5c5ccccc45)cc3)c3ccc4sc5ccccc5c4c3)c2)cc1]

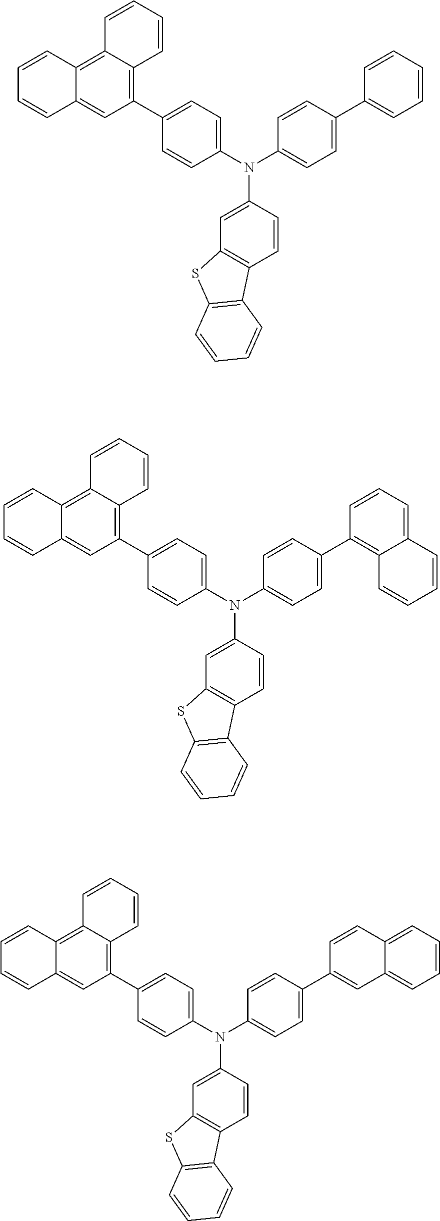 [SMILES: c1ccc(-c2ccc(N(c3ccc(-c4cc5ccccc5c5ccccc45)cc3)c3ccc4c(c3)sc3ccccc34)cc2)cc1 ; c1ccc2c(-c3ccc(N(c4ccc(-c5cc6ccccc6c6ccccc56)cc4)c4ccc5c(c4)sc4ccccc45)cc3)cccc2c1 ; c1ccc2cc(-c3ccc(N(c4ccc(-c5cc6ccccc6c6ccccc56)cc4)c4ccc5c(c4)sc4ccccc45)cc3)ccc2c1]

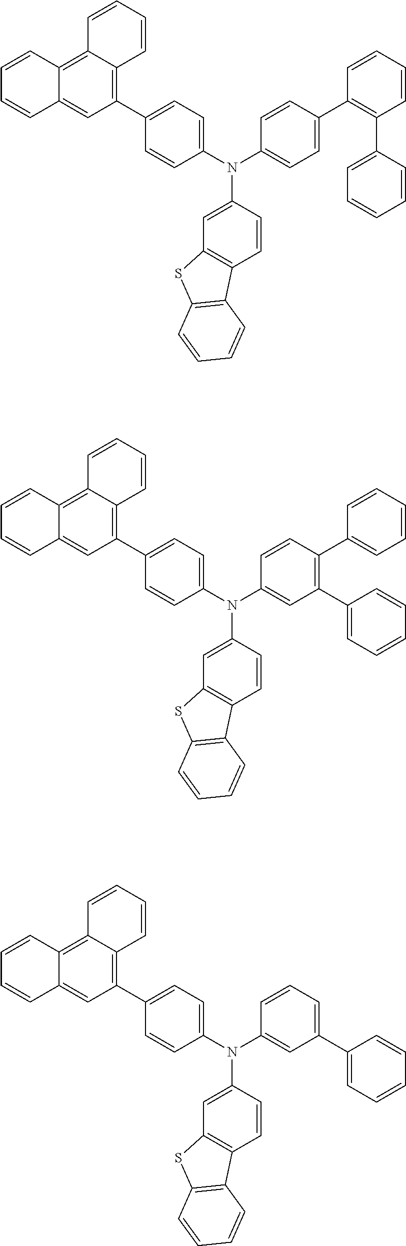 [SMILES: c1ccc(-c2ccccc2-c2ccc(N(c3ccc(-c4cc5ccccc5c5ccccc45)cc3)c3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2-c2ccccc2)cc1 ; c1ccc(-c2cccc(N(c3ccc(-c4cc5ccccc5c5ccccc45)cc3)c3ccc4c(c3)sc3ccccc34)c2)cc1]

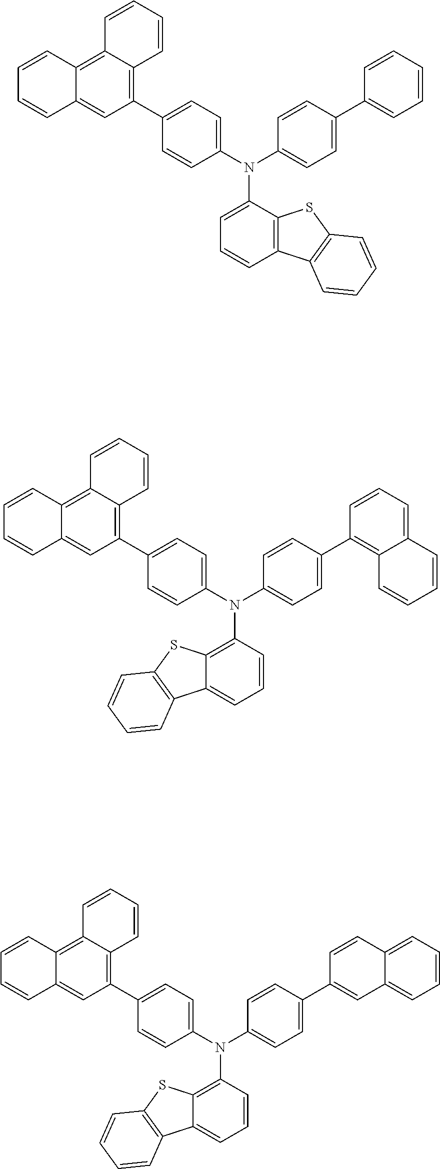 [SMILES: c1ccc(-c2ccc(N(c3ccc(-c4cc5ccccc5c5ccccc45)cc3)c3cccc4c3sc3ccccc34)cc2)cc1 ; c1ccc2c(-c3ccc(N(c4ccc(-c5cc6ccccc6c6ccccc56)cc4)c4cccc5c4sc4ccccc45)cc3)cccc2c1 ; c1ccc2cc(-c3ccc(N(c4ccc(-c5cc6ccccc6c6ccccc56)cc4)c4cccc5c4sc4ccccc45)cc3)ccc2c1]

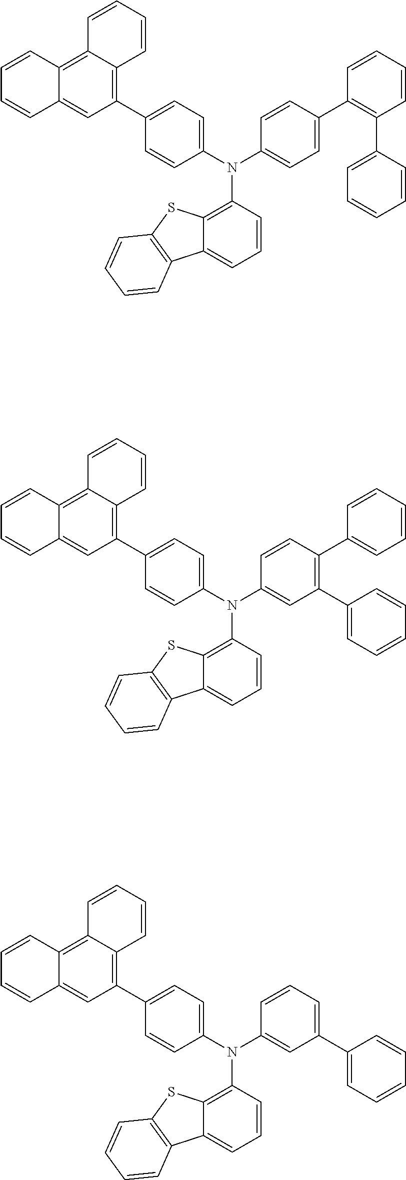 [SMILES: c1ccc(-c2ccccc2-c2ccc(N(c3ccc(-c4cc5ccccc5c5ccccc45)cc3)c3cccc4c3sc3ccccc34)cc2)cc1 ; c1ccc(-c2ccc(N(c3ccc(-c4cc5ccccc5c5ccccc45)cc3)c3cccc4c3sc3ccccc34)cc2-c2ccccc2)cc1 ; c1ccc(-c2cccc(N(c3ccc(-c4cc5ccccc5c5ccccc45)cc3)c3cccc4c3sc3ccccc34)c2)cc1]

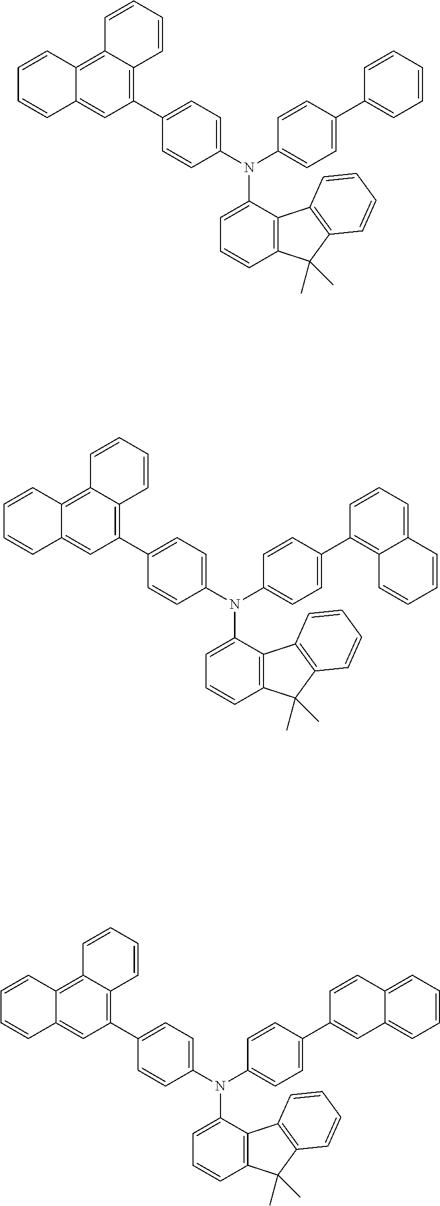 [SMILES: CC1(C)c2ccccc2-c2c(N(c3ccc(-c4ccccc4)cc3)c3ccc(-c4cc5ccccc5c5ccccc45)cc3)cccc21 ; CC1(C)c2ccccc2-c2c(N(c3ccc(-c4cccc5ccccc45)cc3)c3ccc(-c4cc5ccccc5c5ccccc45)cc3)cccc21 ; CC1(C)c2ccccc2-c2c(N(c3ccc(-c4ccc5ccccc5c4)cc3)c3ccc(-c4cc5ccccc5c5ccccc45)cc3)cccc21]

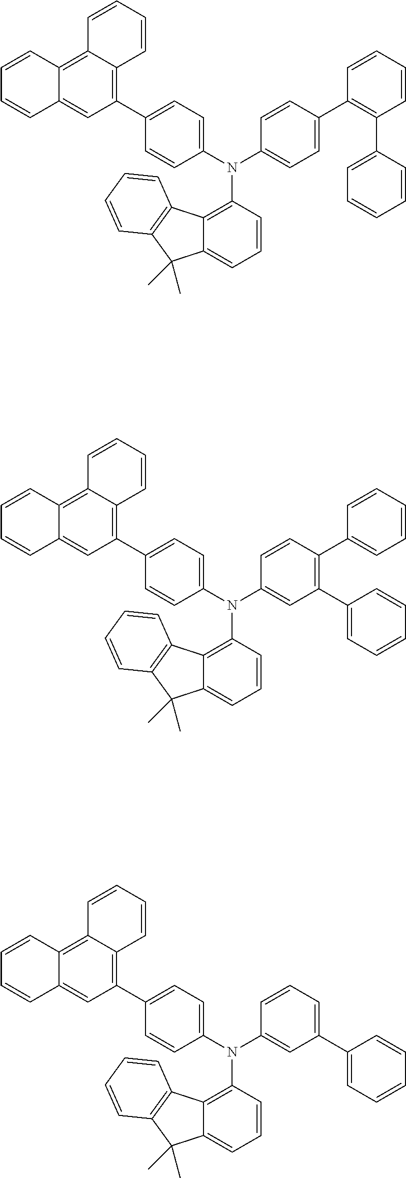 [SMILES: CC1(C)c2ccccc2-c2c(N(c3ccc(-c4ccccc4-c4ccccc4)cc3)c3ccc(-c4cc5ccccc5c5ccccc45)cc3)cccc21 ; CC1(C)c2ccccc2-c2c(N(c3ccc(-c4cc5ccccc5c5ccccc45)cc3)c3ccc(-c4ccccc4)c(-c4ccccc4)c3)cccc21 ; CC1(C)c2ccccc2-c2c(N(c3ccc(-c4cc5ccccc5c5ccccc45)cc3)c3cccc(-c4ccccc4)c3)cccc21]

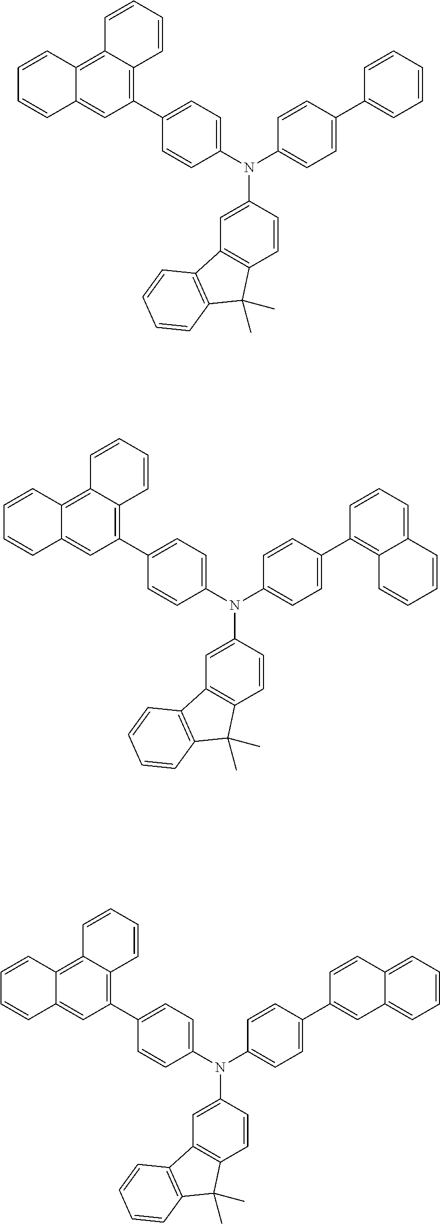 [SMILES: CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4cccc5ccccc45)cc3)c3ccc(-c4cc5ccccc5c5ccccc45)cc3)ccc21 ; CC1(C)c2ccccc2-c2cc(N(c3ccc(-c4ccc5ccccc5c4)cc3)c3ccc(-c4cc5ccccc5c5ccccc45)cc3)ccc21]

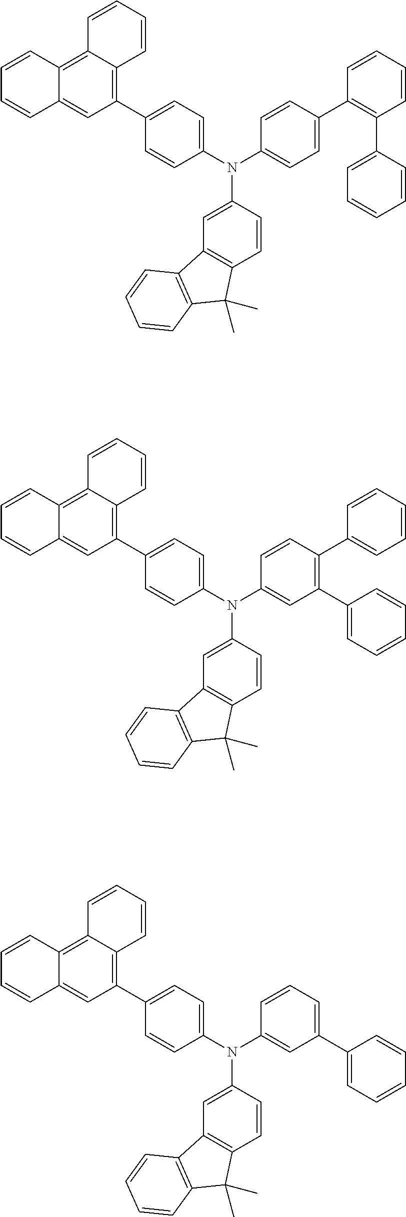 [SMILES: CC1(C)c2ccccc2-c2cc(N(c3ccc(-c4ccccc4-c4ccccc4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3ccc(-c4ccccc4)c(-c4ccccc4)c3)ccc21 ; CC1(C)c2ccccc2-c2cc(N(c3ccc(-c4cc5ccccc5c5ccccc45)cc3)c3cccc(-c4ccccc4)c3)ccc21]

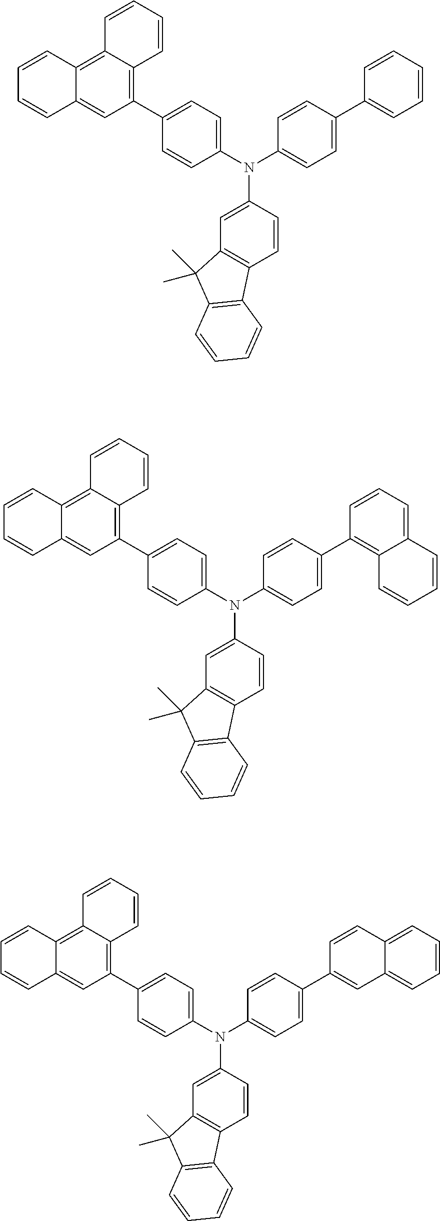 [SMILES: CC1(C)c2ccccc2-c2ccc(N(c3ccc(-c4ccccc4)cc3)c3ccc(-c4cc5ccccc5c5ccccc45)cc3)cc21 ; CC1(C)c2ccccc2-c2ccc(N(c3ccc(-c4cccc5ccccc45)cc3)c3ccc(-c4cc5ccccc5c5ccccc45)cc3)cc21 ; CC1(C)c2ccccc2-c2ccc(N(c3ccc(-c4ccc5ccccc5c4)cc3)c3ccc(-c4cc5ccccc5c5ccccc45)cc3)cc21]

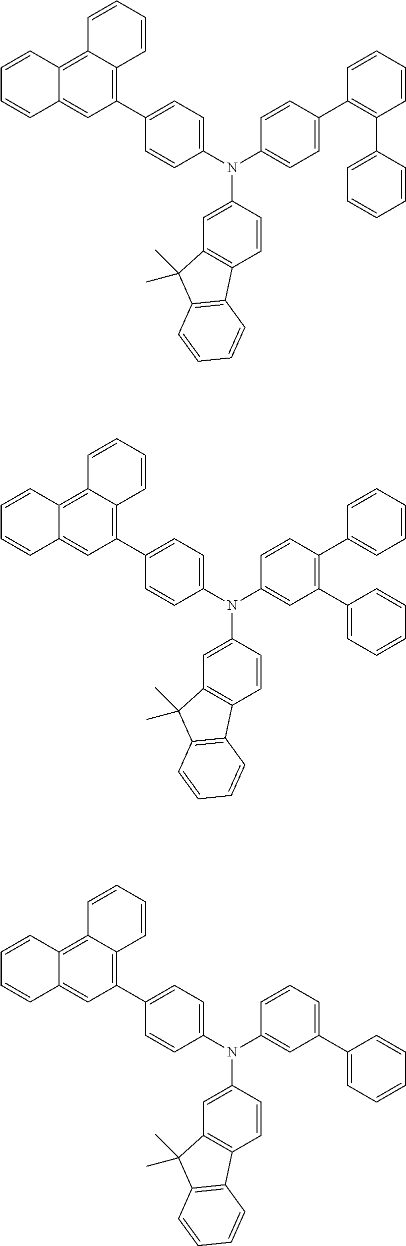 [SMILES: CC1(C)c2ccccc2-c2ccc(N(c3ccc(-c4ccccc4-c4ccccc4)cc3)c3ccc(-c4cc5ccccc5c5ccccc45)cc3)cc21 ; CC1(C)c2ccccc2-c2ccc(N(c3ccc(-c4cc5ccccc5c5ccccc45)cc3)c3ccc(-c4ccccc4)c(-c4ccccc4)c3)cc21 ; CC1(C)c2ccccc2-c2ccc(N(c3ccc(-c4cc5ccccc5c5ccccc45)cc3)c3cccc(-c4ccccc4)c3)cc21]

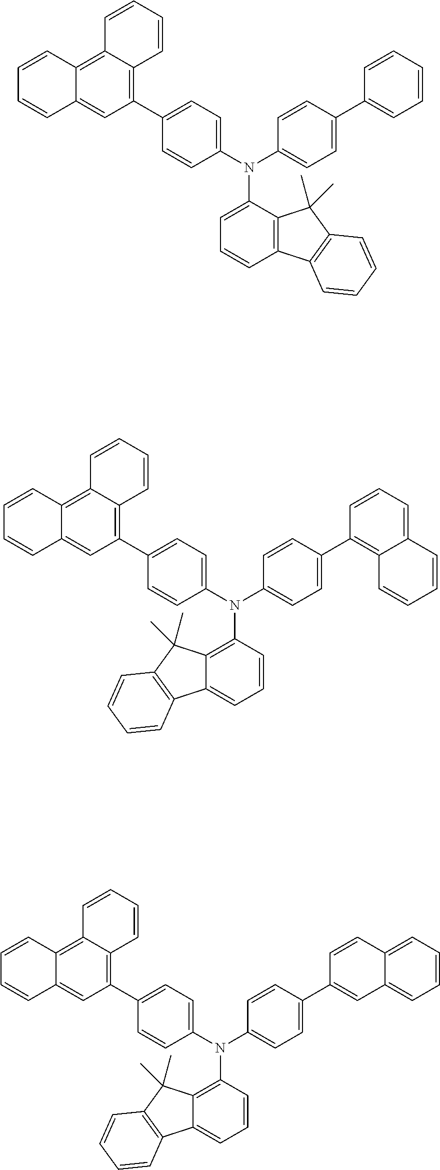 [SMILES: CC1(C)c2ccccc2-c2cccc(N(c3ccc(-c4ccccc4)cc3)c3ccc(-c4cc5ccccc5c5ccccc45)cc3)c21 ; CC1(C)c2ccccc2-c2cccc(N(c3ccc(-c4cccc5ccccc45)cc3)c3ccc(-c4cc5ccccc5c5ccccc45)cc3)c21 ; CC1(C)c2ccccc2-c2cccc(N(c3ccc(-c4ccc5ccccc5c4)cc3)c3ccc(-c4cc5ccccc5c5ccccc45)cc3)c21]

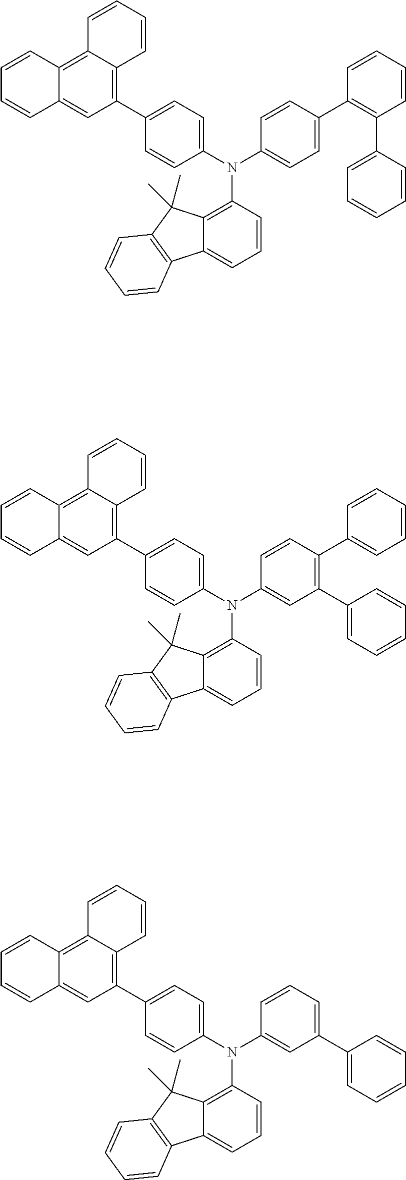 [SMILES: CC1(C)c2ccccc2-c2cccc(N(c3ccc(-c4ccccc4-c4ccccc4)cc3)c3ccc(-c4cc5ccccc5c5ccccc45)cc3)c21 ; CC1(C)c2ccccc2-c2cccc(N(c3ccc(-c4cc5ccccc5c5ccccc45)cc3)c3ccc(-c4ccccc4)c(-c4ccccc4)c3)c21 ; CC1(C)c2ccccc2-c2cccc(N(c3ccc(-c4cc5ccccc5c5ccccc45)cc3)c3cccc(-c4ccccc4)c3)c21]

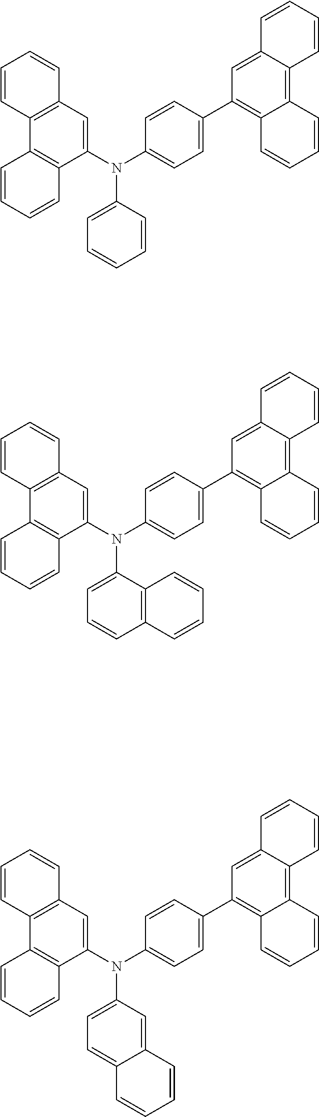 [SMILES: c1ccc(N(c2ccc(-c3cc4ccccc4c4ccccc34)cc2)c2cc3ccccc3c3ccccc23)cc1 ; c1ccc2c(N(c3ccc(-c4cc5ccccc5c5ccccc45)cc3)c3cc4ccccc4c4ccccc34)cccc2c1 ; c1ccc2cc(N(c3ccc(-c4cc5ccccc5c5ccccc45)cc3)c3cc4ccccc4c4ccccc34)ccc2c1]

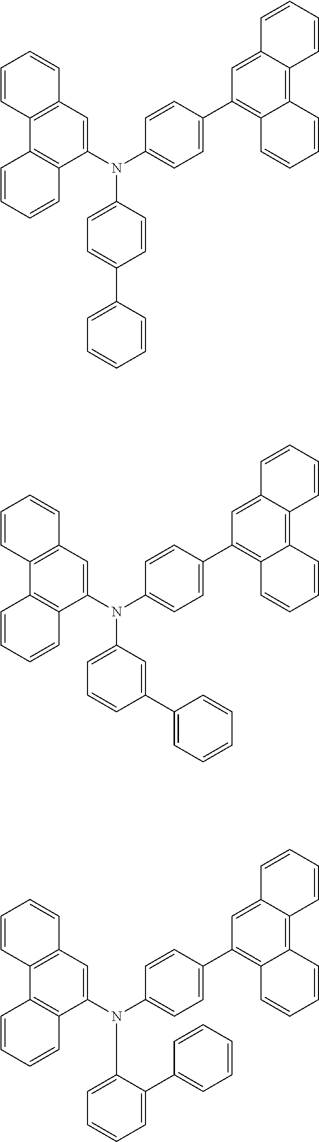 [SMILES: c1ccc(-c2ccc(N(c3ccc(-c4cc5ccccc5c5ccccc45)cc3)c3cc4ccccc4c4ccccc34)cc2)cc1 ; c1ccc(-c2cccc(N(c3ccc(-c4cc5ccccc5c5ccccc45)cc3)c3cc4ccccc4c4ccccc34)c2)cc1 ; c1ccc(-c2ccccc2N(c2ccc(-c3cc4ccccc4c4ccccc34)cc2)c2cc3ccccc3c3ccccc23)cc1]

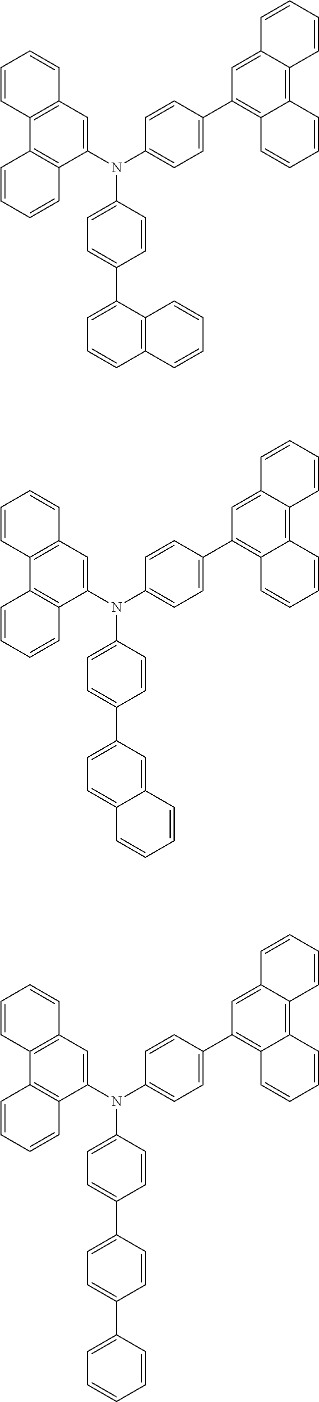 [SMILES: c1ccc2c(-c3ccc(N(c4ccc(-c5cc6ccccc6c6ccccc56)cc4)c4cc5ccccc5c5ccccc45)cc3)cccc2c1 ; c1ccc2cc(-c3ccc(N(c4ccc(-c5cc6ccccc6c6ccccc56)cc4)c4cc5ccccc5c5ccccc45)cc3)ccc2c1 ; c1ccc(-c2ccc(-c3ccc(N(c4ccc(-c5cc6ccccc6c6ccccc56)cc4)c4cc5ccccc5c5ccccc45)cc3)cc2)cc1]

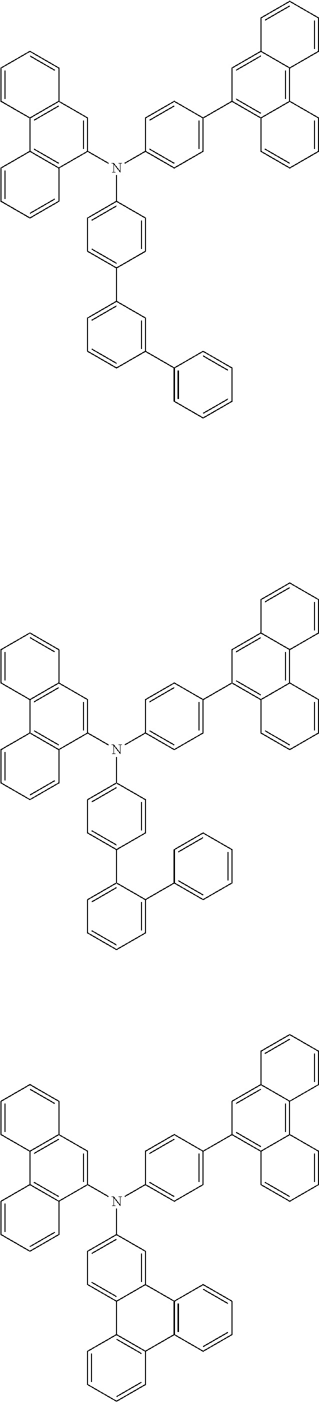 [SMILES: c1ccc(-c2cccc(-c3ccc(N(c4ccc(-c5cc6ccccc6c6ccccc56)cc4)c4cc5ccccc5c5ccccc45)cc3)c2)cc1 ; c1ccc(-c2ccccc2-c2ccc(N(c3ccc(-c4cc5ccccc5c5ccccc45)cc3)c3cc4ccccc4c4ccccc34)cc2)cc1 ; c1ccc2c(c1)cc(-c1ccc(N(c3ccc4c5ccccc5c5ccccc5c4c3)c3cc4ccccc4c4ccccc34)cc1)c1ccccc12]

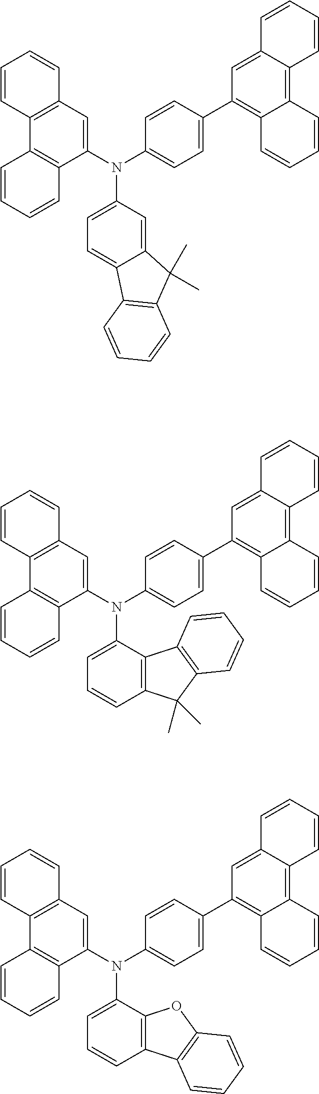 [SMILES: CC1(C)c2ccccc2-c2ccc(N(c3ccc(-c4cc5ccccc5c5ccccc45)cc3)c3cc4ccccc4c4ccccc34)cc21 ; CC1(C)c2ccccc2-c2c(N(c3ccc(-c4cc5ccccc5c5ccccc45)cc3)c3cc4ccccc4c4ccccc34)cccc21 ; c1ccc2c(c1)cc(-c1ccc(N(c3cc4ccccc4c4ccccc34)c3cccc4c3oc3ccccc34)cc1)c1ccccc12]

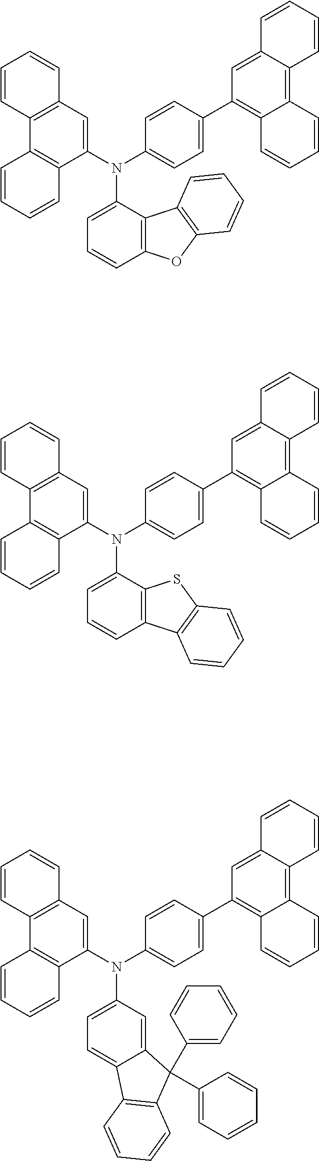 [SMILES: c1ccc2c(c1)cc(-c1ccc(N(c3cc4ccccc4c4ccccc34)c3cccc4oc5ccccc5c34)cc1)c1ccccc12 ; c1ccc2c(c1)cc(-c1ccc(N(c3cc4ccccc4c4ccccc34)c3cccc4c3sc3ccccc34)cc1)c1ccccc12 ; c1ccc(C2(c3ccccc3)c3ccccc3-c3ccc(N(c4ccc(-c5cc6ccccc6c6ccccc56)cc4)c4cc5ccccc5c5ccccc45)cc32)cc1]

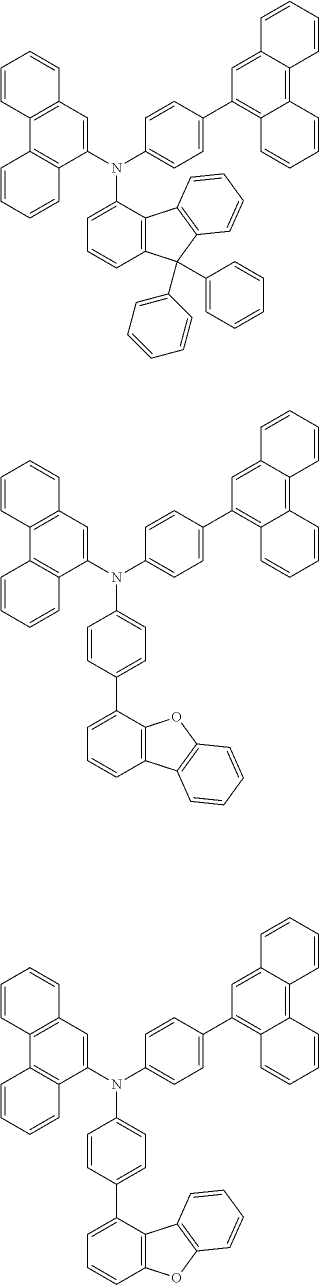 [SMILES: c1ccc(C2(c3ccccc3)c3ccccc3-c3c(N(c4ccc(-c5cc6ccccc6c6ccccc56)cc4)c4cc5ccccc5c5ccccc45)cccc32)cc1 ; c1ccc2c(c1)cc(-c1ccc(N(c3ccc(-c4cccc5c4oc4ccccc45)cc3)c3cc4ccccc4c4ccccc34)cc1)c1ccccc12 ; c1ccc2c(c1)cc(-c1ccc(N(c3ccc(-c4cccc5oc6ccccc6c45)cc3)c3cc4ccccc4c4ccccc34)cc1)c1ccccc12]

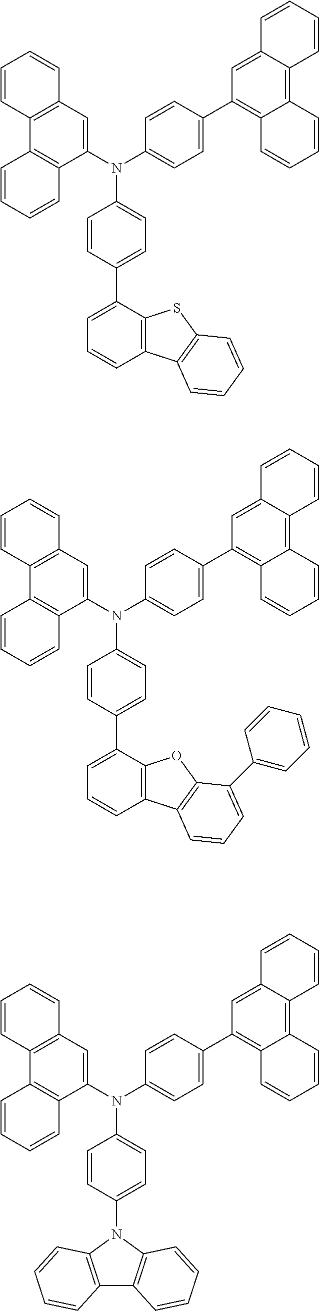 [SMILES: c1ccc2c(c1)cc(-c1ccc(N(c3ccc(-c4cccc5c4sc4ccccc45)cc3)c3cc4ccccc4c4ccccc34)cc1)c1ccccc12 ; c1ccc(-c2cccc3c2oc2c(-c4ccc(N(c5ccc(-c6cc7ccccc7c7ccccc67)cc5)c5cc6ccccc6c6ccccc56)cc4)cccc23)cc1 ; c1ccc2c(c1)cc(-c1ccc(N(c3ccc(-n4c5ccccc5c5ccccc54)cc3)c3cc4ccccc4c4ccccc34)cc1)c1ccccc12]

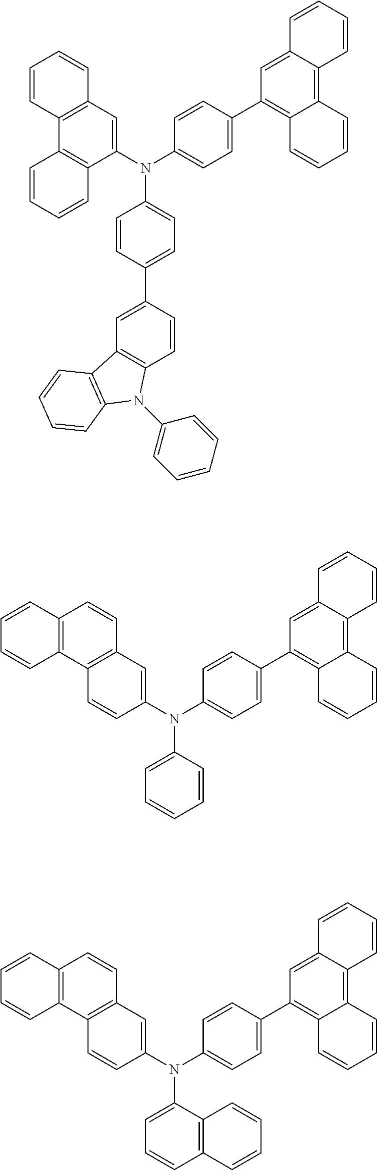 [SMILES: c1ccc(-n2c3ccccc3c3cc(-c4ccc(N(c5ccc(-c6cc7ccccc7c7ccccc67)cc5)c5cc6ccccc6c6ccccc56)cc4)ccc32)cc1 ; c1ccc(N(c2ccc(-c3cc4ccccc4c4ccccc34)cc2)c2ccc3c(ccc4ccccc43)c2)cc1 ; c1ccc2c(N(c3ccc(-c4cc5ccccc5c5ccccc45)cc3)c3ccc4c(ccc5ccccc54)c3)cccc2c1]

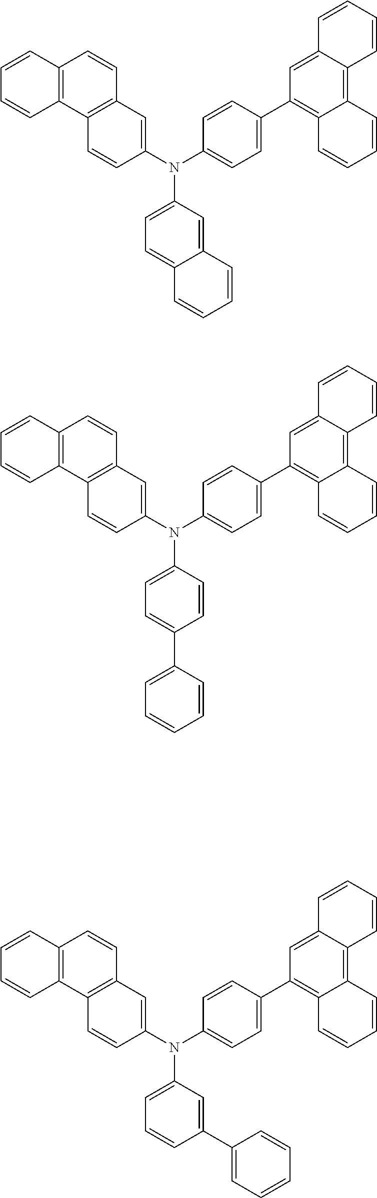 [SMILES: c1ccc2cc(N(c3ccc(-c4cc5ccccc5c5ccccc45)cc3)c3ccc4c(ccc5ccccc54)c3)ccc2c1 ; c1ccc(-c2ccc(N(c3ccc(-c4cc5ccccc5c5ccccc45)cc3)c3ccc4c(ccc5ccccc54)c3)cc2)cc1 ; c1ccc(-c2cccc(N(c3ccc(-c4cc5ccccc5c5ccccc45)cc3)c3ccc4c(ccc5ccccc54)c3)c2)cc1]

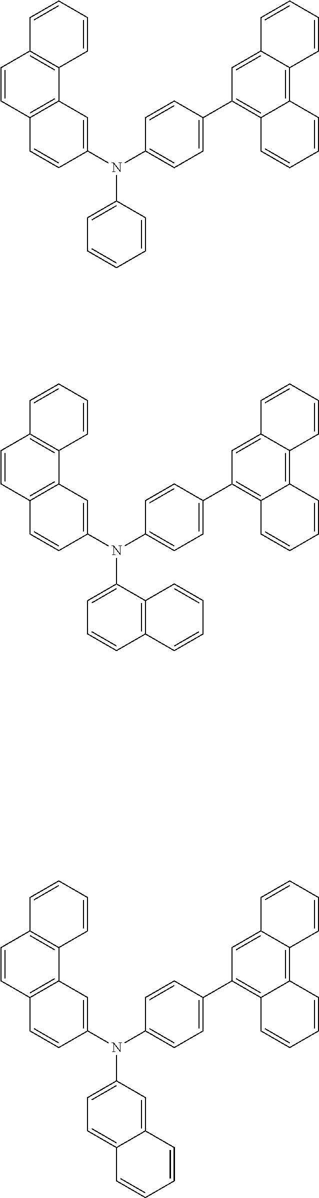 [SMILES: c1ccc(N(c2ccc(-c3cc4ccccc4c4ccccc34)cc2)c2ccc3ccc4ccccc4c3c2)cc1 ; c1ccc2c(N(c3ccc(-c4cc5ccccc5c5ccccc45)cc3)c3ccc4ccc5ccccc5c4c3)cccc2c1 ; c1ccc2cc(N(c3ccc(-c4cc5ccccc5c5ccccc45)cc3)c3ccc4ccc5ccccc5c4c3)ccc2c1]

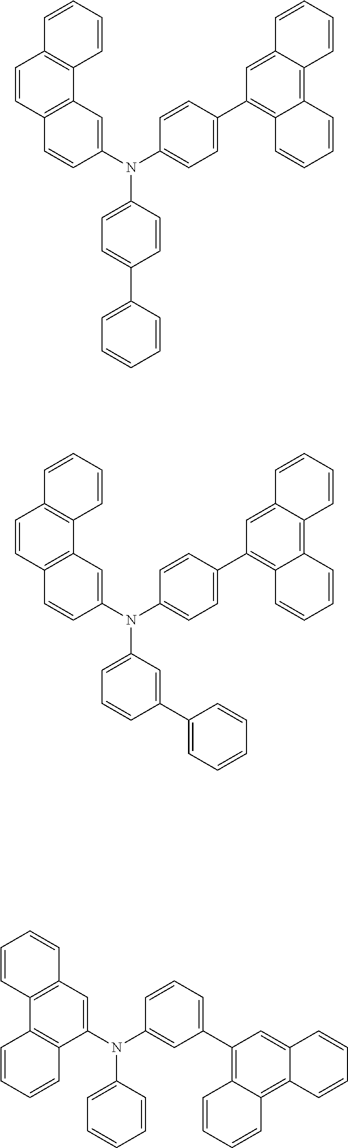 [SMILES: c1ccc(-c2ccc(N(c3ccc(-c4cc5ccccc5c5ccccc45)cc3)c3ccc4ccc5ccccc5c4c3)cc2)cc1 ; c1ccc(-c2cccc(N(c3ccc(-c4cc5ccccc5c5ccccc45)cc3)c3ccc4ccc5ccccc5c4c3)c2)cc1 ; c1ccc(N(c2cccc(-c3cc4ccccc4c4ccccc34)c2)c2cc3ccccc3c3ccccc23)cc1]

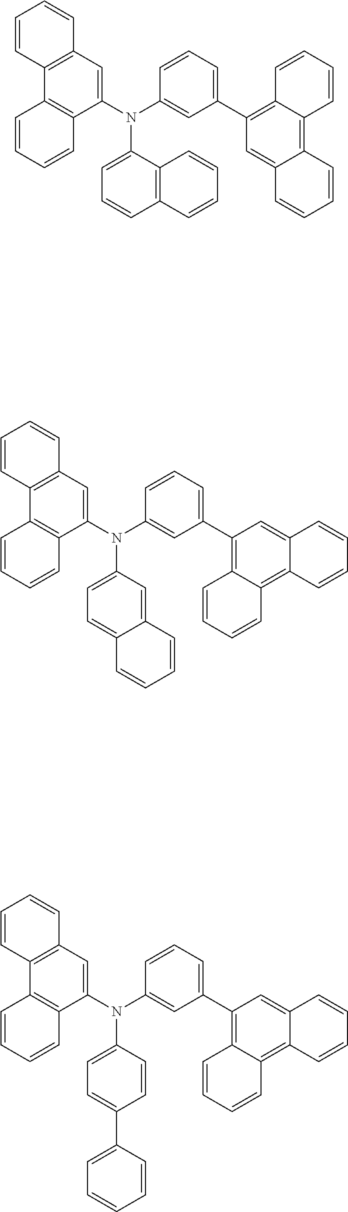 [SMILES: c1cc(-c2cc3ccccc3c3ccccc23)cc(N(c2cccc3ccccc23)c2cc3ccccc3c3ccccc23)c1 ; c1cc(-c2cc3ccccc3c3ccccc23)cc(N(c2ccc3ccccc3c2)c2cc3ccccc3c3ccccc23)c1 ; c1ccc(-c2ccc(N(c3cccc(-c4cc5ccccc5c5ccccc45)c3)c3cc4ccccc4c4ccccc34)cc2)cc1]

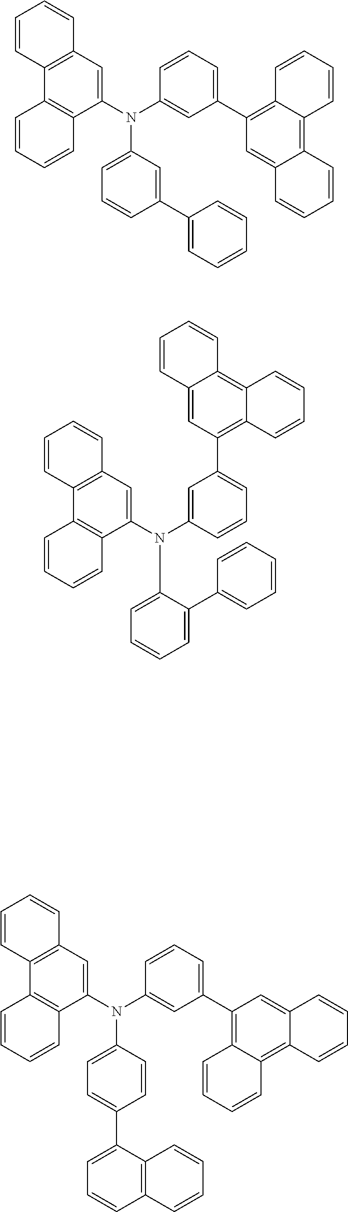 [SMILES: c1ccc(-c2cccc(N(c3cccc(-c4cc5ccccc5c5ccccc45)c3)c3cc4ccccc4c4ccccc34)c2)cc1 ; c1ccc(-c2ccccc2N(c2cccc(-c3cc4ccccc4c4ccccc34)c2)c2cc3ccccc3c3ccccc23)cc1 ; c1cc(-c2cc3ccccc3c3ccccc23)cc(N(c2ccc(-c3cccc4ccccc34)cc2)c2cc3ccccc3c3ccccc23)c1]

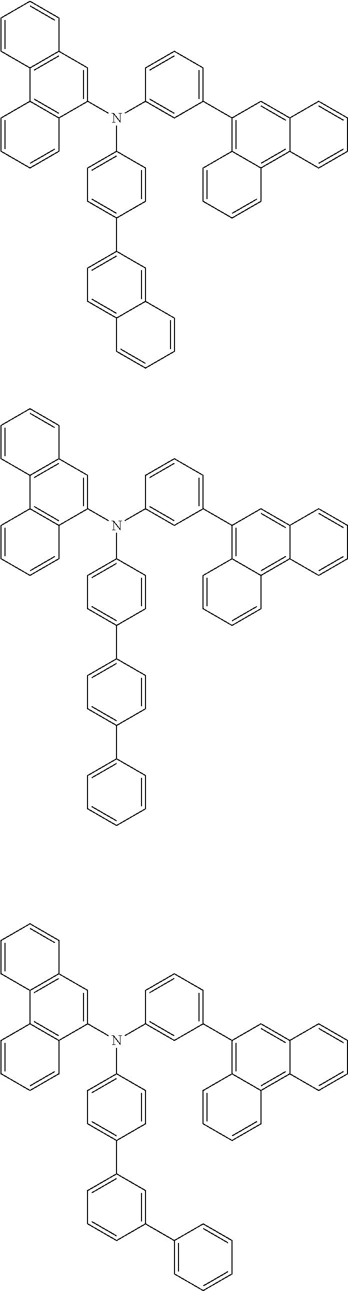 [SMILES: c1cc(-c2cc3ccccc3c3ccccc23)cc(N(c2ccc(-c3ccc4ccccc4c3)cc2)c2cc3ccccc3c3ccccc23)c1 ; c1ccc(-c2ccc(-c3ccc(N(c4cccc(-c5cc6ccccc6c6ccccc56)c4)c4cc5ccccc5c5ccccc45)cc3)cc2)cc1 ; c1ccc(-c2cccc(-c3ccc(N(c4cccc(-c5cc6ccccc6c6ccccc56)c4)c4cc5ccccc5c5ccccc45)cc3)c2)cc1]

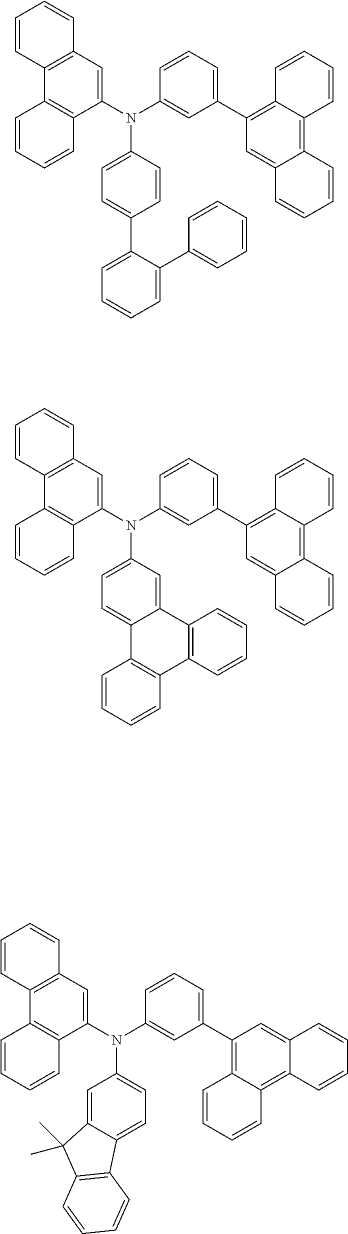 [SMILES: c1ccc(-c2ccccc2-c2ccc(N(c3cccc(-c4cc5ccccc5c5ccccc45)c3)c3cc4ccccc4c4ccccc34)cc2)cc1 ; c1cc(-c2cc3ccccc3c3ccccc23)cc(N(c2ccc3c4ccccc4c4ccccc4c3c2)c2cc3ccccc3c3ccccc23)c1 ; CC1(C)c2ccccc2-c2ccc(N(c3cccc(-c4cc5ccccc5c5ccccc45)c3)c3cc4ccccc4c4ccccc34)cc21]

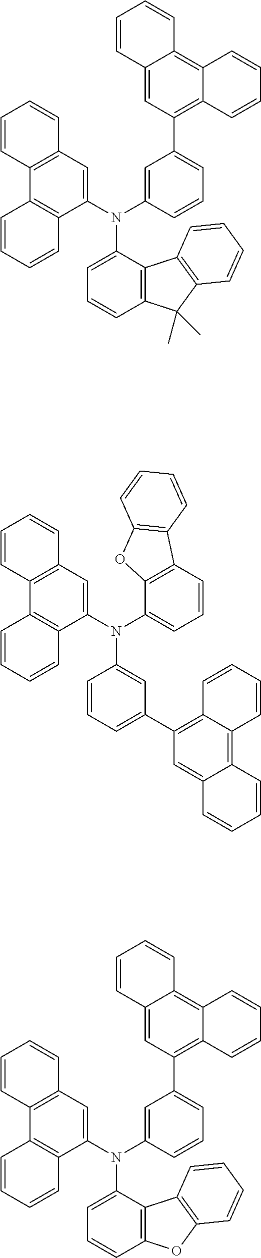 [SMILES: CC1(C)c2ccccc2-c2c(N(c3cccc(-c4cc5ccccc5c5ccccc45)c3)c3cc4ccccc4c4ccccc34)cccc21 ; c1cc(-c2cc3ccccc3c3ccccc23)cc(N(c2cc3ccccc3c3ccccc23)c2cccc3c2oc2ccccc23)c1 ; c1cc(-c2cc3ccccc3c3ccccc23)cc(N(c2cc3ccccc3c3ccccc23)c2cccc3oc4ccccc4c23)c1]

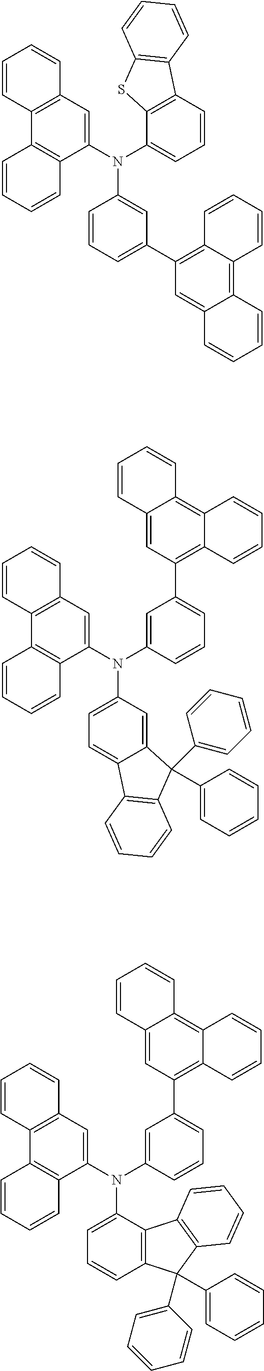 [SMILES: c1cc(-c2cc3ccccc3c3ccccc23)cc(N(c2cc3ccccc3c3ccccc23)c2cccc3c2sc2ccccc23)c1 ; c1ccc(C2(c3ccccc3)c3ccccc3-c3ccc(N(c4cccc(-c5cc6ccccc6c6ccccc56)c4)c4cc5ccccc5c5ccccc45)cc32)cc1 ; c1ccc(C2(c3ccccc3)c3ccccc3-c3c(N(c4cccc(-c5cc6ccccc6c6ccccc56)c4)c4cc5ccccc5c5ccccc45)cccc32)cc1]

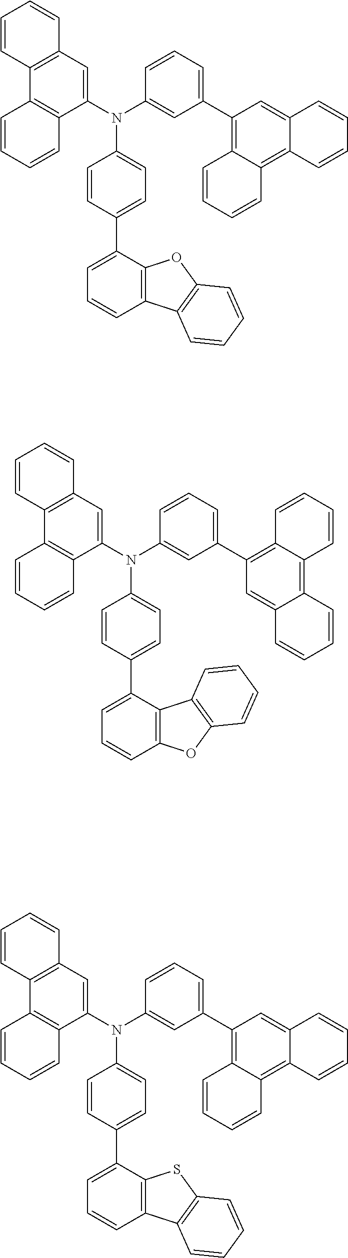 [SMILES: c1cc(-c2cc3ccccc3c3ccccc23)cc(N(c2ccc(-c3cccc4c3oc3ccccc34)cc2)c2cc3ccccc3c3ccccc23)c1 ; c1cc(-c2cc3ccccc3c3ccccc23)cc(N(c2ccc(-c3cccc4oc5ccccc5c34)cc2)c2cc3ccccc3c3ccccc23)c1 ; c1cc(-c2cc3ccccc3c3ccccc23)cc(N(c2ccc(-c3cccc4c3sc3ccccc34)cc2)c2cc3ccccc3c3ccccc23)c1]

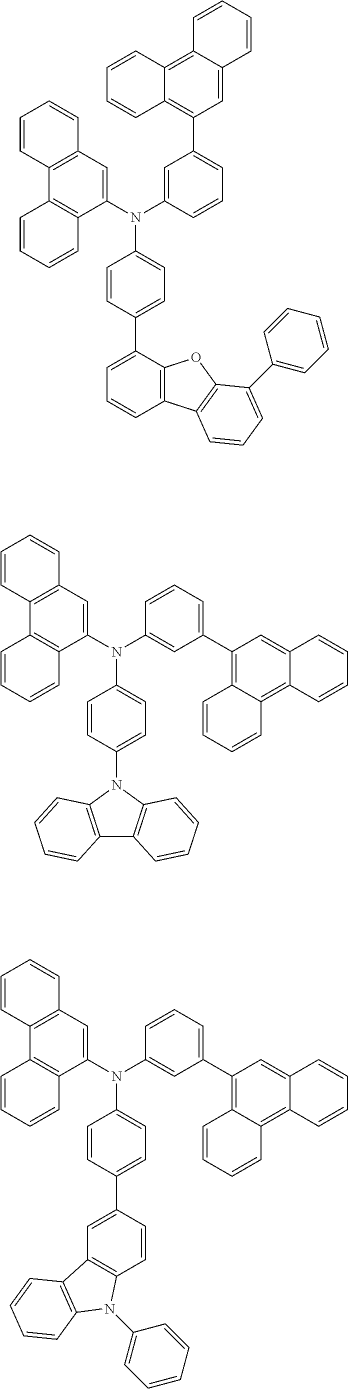 [SMILES: c1ccc(-c2cccc3c2oc2c(-c4ccc(N(c5cccc(-c6cc7ccccc7c7ccccc67)c5)c5cc6ccccc6c6ccccc56)cc4)cccc23)cc1 ; c1cc(-c2cc3ccccc3c3ccccc23)cc(N(c2ccc(-n3c4ccccc4c4ccccc43)cc2)c2cc3ccccc3c3ccccc23)c1 ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1]

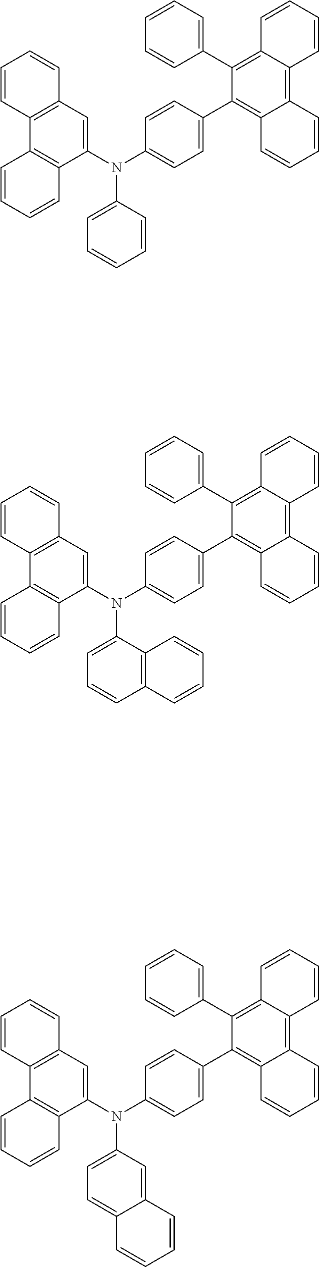 [SMILES: c1ccc(-c2c(-c3ccc(N(c4ccccc4)c4cc5ccccc5c5ccccc45)cc3)c3ccccc3c3ccccc23)cc1 ; c1ccc(-c2c(-c3ccc(N(c4cccc5ccccc45)c4cc5ccccc5c5ccccc45)cc3)c3ccccc3c3ccccc23)cc1 ; c1ccc(-c2c(-c3ccc(N(c4ccc5ccccc5c4)c4cc5ccccc5c5ccccc45)cc3)c3ccccc3c3ccccc23)cc1]

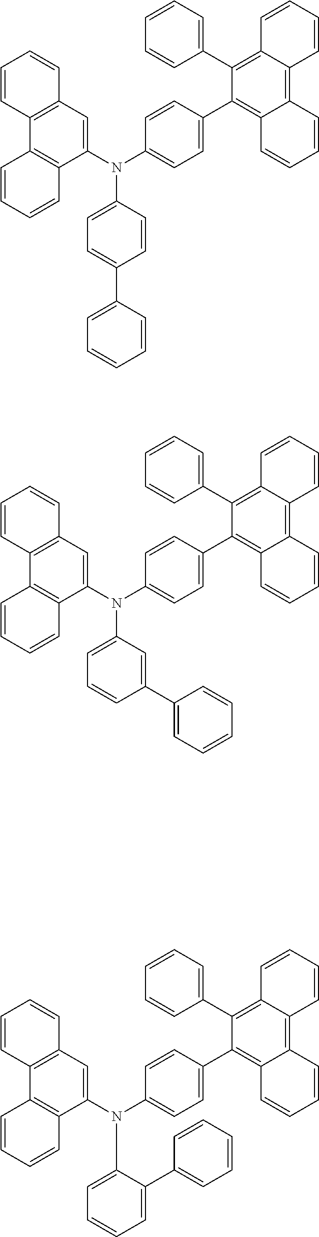 [SMILES: c1ccc(-c2ccc(N(c3ccc(-c4c(-c5ccccc5)c5ccccc5c5ccccc45)cc3)c3cc4ccccc4c4ccccc34)cc2)cc1 ; c1ccc(-c2cccc(N(c3ccc(-c4c(-c5ccccc5)c5ccccc5c5ccccc45)cc3)c3cc4ccccc4c4ccccc34)c2)cc1 ; c1ccc(-c2ccccc2N(c2ccc(-c3c(-c4ccccc4)c4ccccc4c4ccccc34)cc2)c2cc3ccccc3c3ccccc23)cc1]

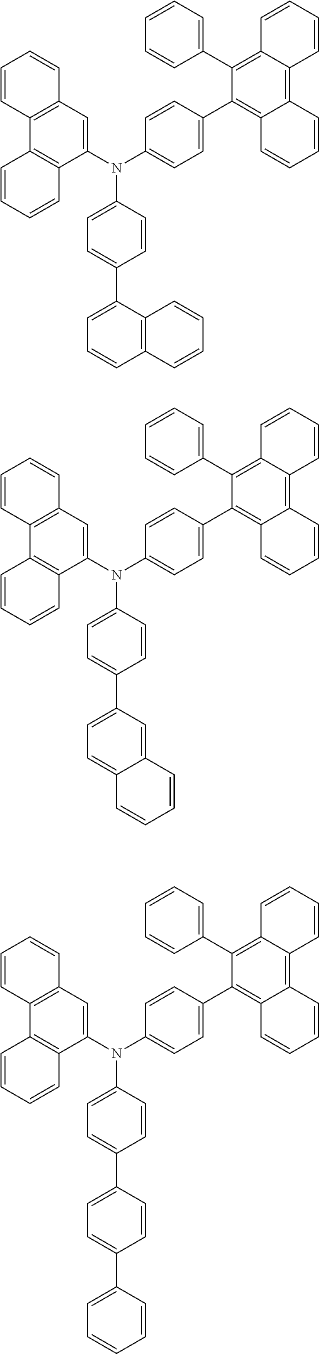

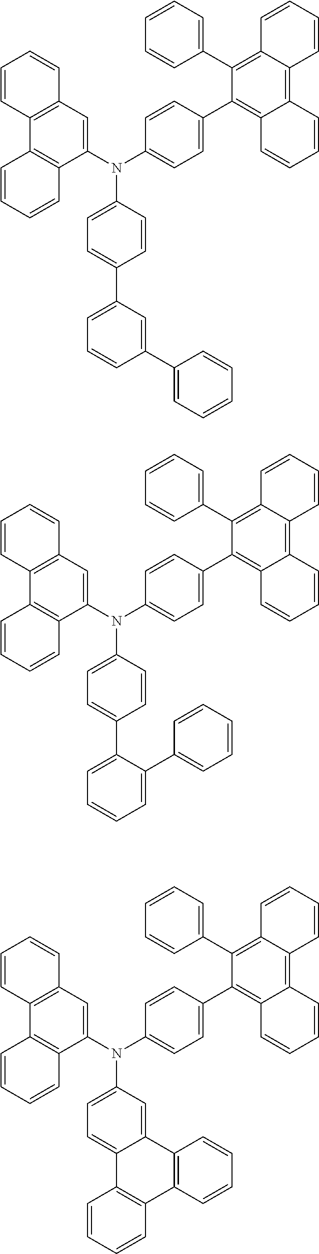

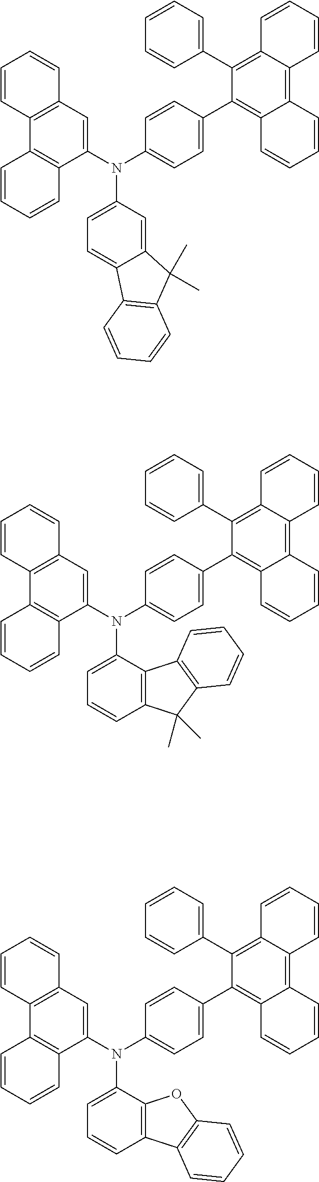 [SMILES: CC1(C)c2ccccc2-c2ccc(N(c3ccc(-c4c(-c5ccccc5)c5ccccc5c5ccccc45)cc3)c3cc4ccccc4c4ccccc34)cc21 ; CC1(C)c2ccccc2-c2c(N(c3ccc(-c4c(-c5ccccc5)c5ccccc5c5ccccc45)cc3)c3cc4ccccc4c4ccccc34)cccc21 ; c1ccc(-c2c(-c3ccc(N(c4cc5ccccc5c5ccccc45)c4cccc5c4oc4ccccc45)cc3)c3ccccc3c3ccccc23)cc1]

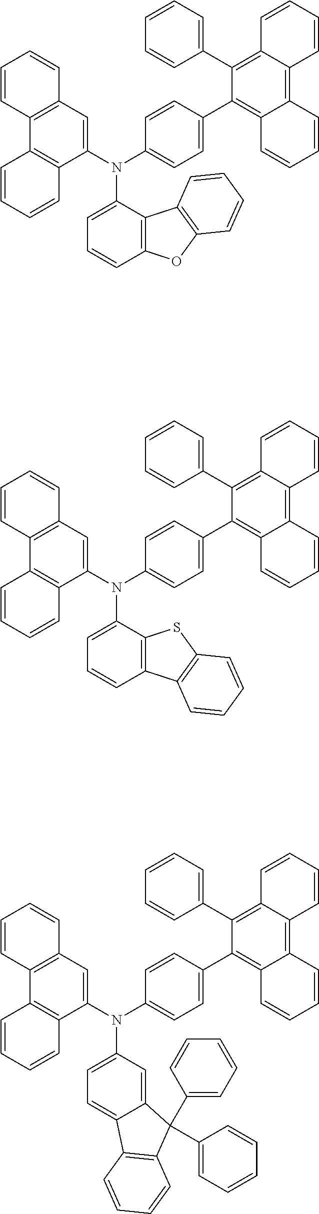 [SMILES: c1ccc(-c2c(-c3ccc(N(c4cc5ccccc5c5ccccc45)c4cccc5oc6ccccc6c45)cc3)c3ccccc3c3ccccc23)cc1 ; c1ccc(-c2c(-c3ccc(N(c4cc5ccccc5c5ccccc45)c4cccc5c4sc4ccccc45)cc3)c3ccccc3c3ccccc23)cc1 ; c1ccc(-c2c(-c3ccc(N(c4ccc5c(c4)C(c4ccccc4)(c4ccccc4)c4ccccc4-5)c4cc5ccccc5c5ccccc45)cc3)c3ccccc3c3ccccc23)cc1]

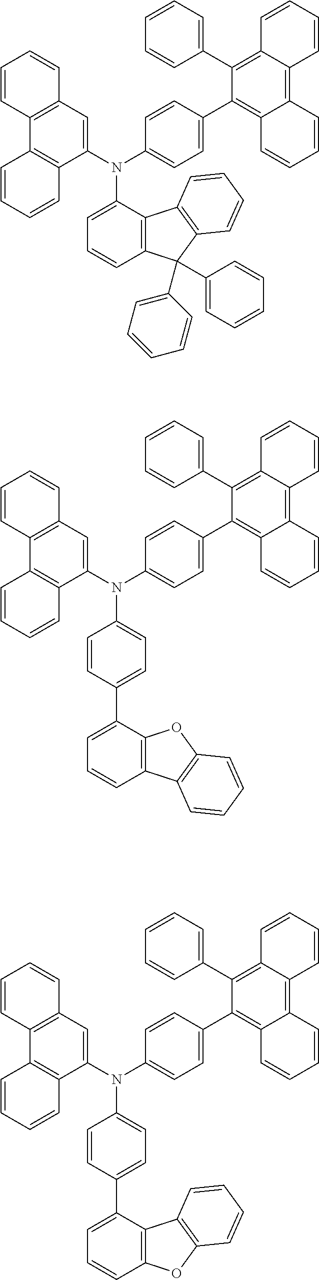 [SMILES: c1ccc(-c2c(-c3ccc(N(c4cccc5c4-c4ccccc4C5(c4ccccc4)c4ccccc4)c4cc5ccccc5c5ccccc45)cc3)c3ccccc3c3ccccc23)cc1 ; c1ccc(-c2c(-c3ccc(N(c4ccc(-c5cccc6c5oc5ccccc56)cc4)c4cc5ccccc5c5ccccc45)cc3)c3ccccc3c3ccccc23)cc1 ; c1ccc(-c2c(-c3ccc(N(c4ccc(-c5cccc6oc7ccccc7c56)cc4)c4cc5ccccc5c5ccccc45)cc3)c3ccccc3c3ccccc23)cc1]

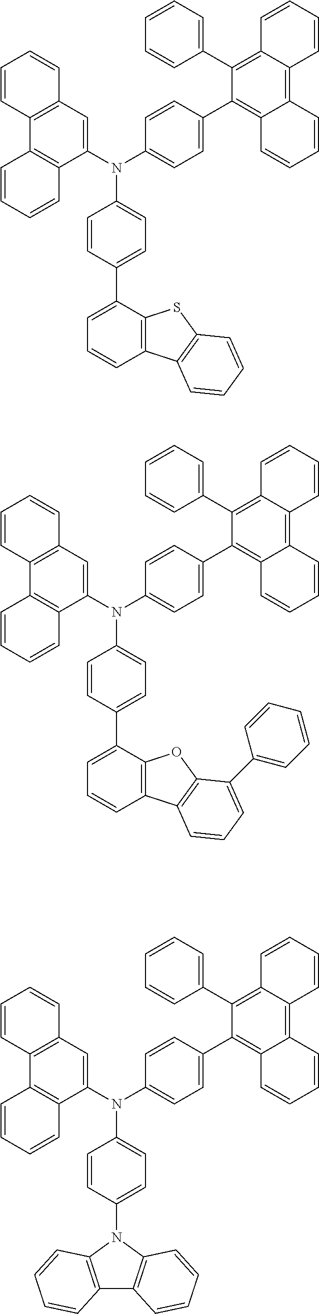 [SMILES: c1ccc(-c2c(-c3ccc(N(c4ccc(-c5cccc6c5sc5ccccc56)cc4)c4cc5ccccc5c5ccccc45)cc3)c3ccccc3c3ccccc23)cc1 ; c1ccc(-c2c(-c3ccc(N(c4ccc(-c5cccc6c5oc5c(-c7ccccc7)cccc56)cc4)c4cc5ccccc5c5ccccc45)cc3)c3ccccc3c3ccccc23)cc1 ; c1ccc(-c2c(-c3ccc(N(c4ccc(-n5c6ccccc6c6ccccc65)cc4)c4cc5ccccc5c5ccccc45)cc3)c3ccccc3c3ccccc23)cc1]

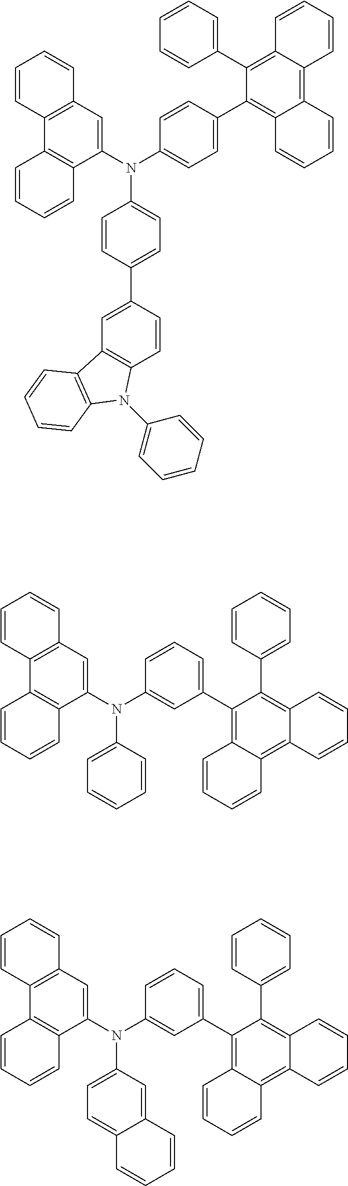 [SMILES: c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1 ; c1ccc(-c2c(-c3cccc(N(c4ccccc4)c4cc5ccccc5c5ccccc45)c3)c3ccccc3c3ccccc23)cc1 ; c1ccc(-c2c(-c3cccc(N(c4ccc5ccccc5c4)c4cc5ccccc5c5ccccc45)c3)c3ccccc3c3ccccc23)cc1]

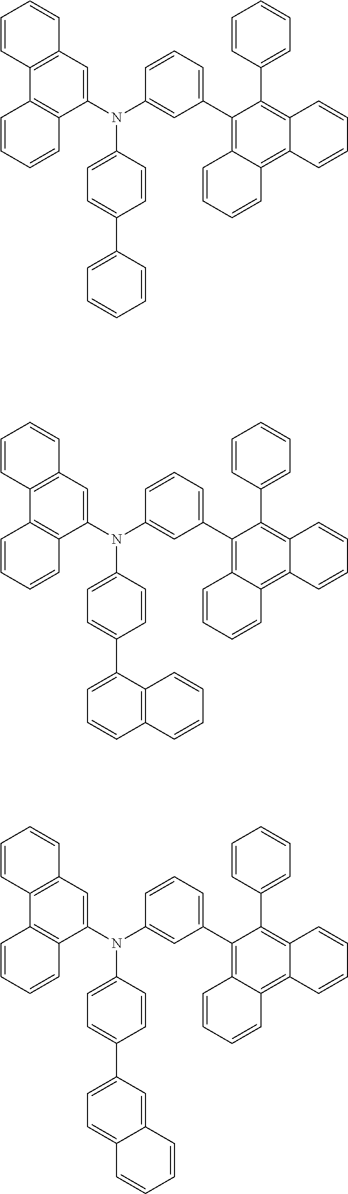 [SMILES: c1ccc(-c2ccc(N(c3cccc(-c4c(-c5ccccc5)c5ccccc5c5ccccc45)c3)c3cc4ccccc4c4ccccc34)cc2)cc1 ; c1ccc(-c2c(-c3cccc(N(c4ccc(-c5cccc6ccccc56)cc4)c4cc5ccccc5c5ccccc45)c3)c3ccccc3c3ccccc23)cc1 ; c1ccc(-c2c(-c3cccc(N(c4ccc(-c5ccc6ccccc6c5)cc4)c4cc5ccccc5c5ccccc45)c3)c3ccccc3c3ccccc23)cc1]

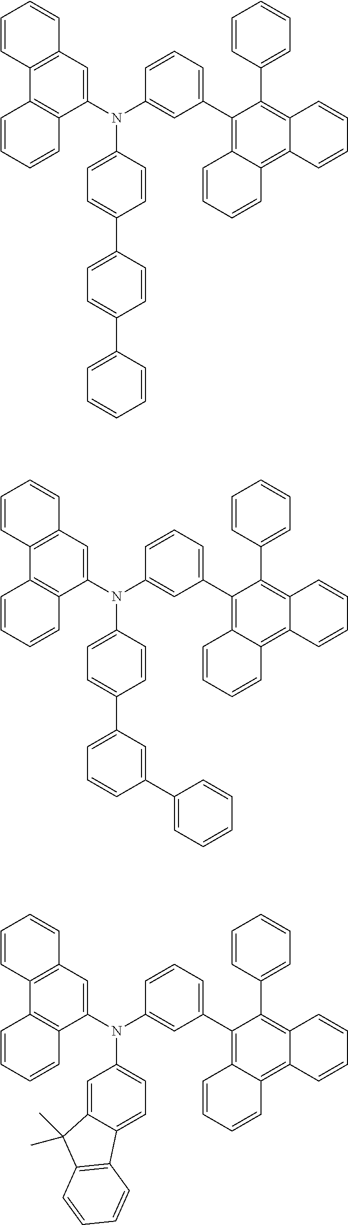 [SMILES: c1ccc(-c2ccc(-c3ccc(N(c4cccc(-c5c(-c6ccccc6)c6ccccc6c6ccccc56)c4)c4cc5ccccc5c5ccccc45)cc3)cc2)cc1 ; c1ccc(-c2cccc(-c3ccc(N(c4cccc(-c5c(-c6ccccc6)c6ccccc6c6ccccc56)c4)c4cc5ccccc5c5ccccc45)cc3)c2)cc1 ; CC1(C)c2ccccc2-c2ccc(N(c3cccc(-c4c(-c5ccccc5)c5ccccc5c5ccccc45)c3)c3cc4ccccc4c4ccccc34)cc21]

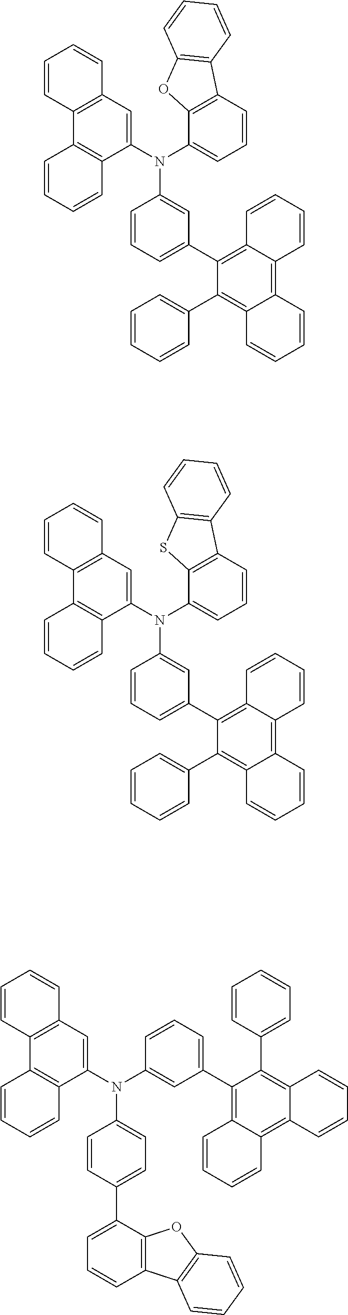 [SMILES: c1ccc(-c2c(-c3cccc(N(c4cc5ccccc5c5ccccc45)c4cccc5c4oc4ccccc45)c3)c3ccccc3c3ccccc23)cc1 ; c1ccc(-c2c(-c3cccc(N(c4cc5ccccc5c5ccccc45)c4cccc5c4sc4ccccc45)c3)c3ccccc3c3ccccc23)cc1 ; c1ccc(-c2c(-c3cccc(N(c4ccc(-c5cccc6c5oc5ccccc56)cc4)c4cc5ccccc5c5ccccc45)c3)c3ccccc3c3ccccc23)cc1]

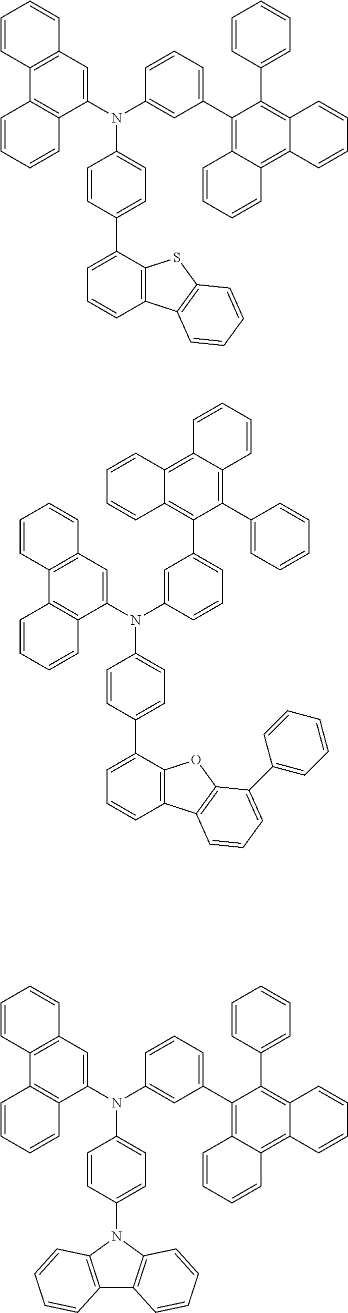 [SMILES: c1ccc(-c2c(-c3cccc(N(c4ccc(-c5cccc6c5sc5ccccc56)cc4)c4cc5ccccc5c5ccccc45)c3)c3ccccc3c3ccccc23)cc1 ; c1ccc(-c2c(-c3cccc(N(c4ccc(-c5cccc6c5oc5c(-c7ccccc7)cccc56)cc4)c4cc5ccccc5c5ccccc45)c3)c3ccccc3c3ccccc23)cc1 ; c1ccc(-c2c(-c3cccc(N(c4ccc(-n5c6ccccc6c6ccccc65)cc4)c4cc5ccccc5c5ccccc45)c3)c3ccccc3c3ccccc23)cc1]

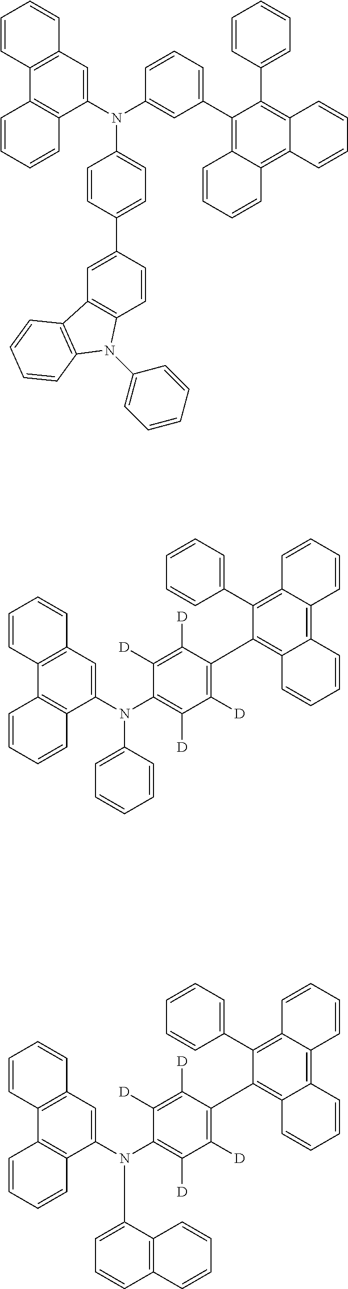 [SMILES: c1ccc(-c2c(-c3cccc(N(c4ccc(-c5ccc6c(c5)c5ccccc5n6-c5ccccc5)cc4)c4cc5ccccc5c5ccccc45)c3)c3ccccc3c3ccccc23)cc1 ; [2H]c1c([2H])c(N(c2ccccc2)c2cc3ccccc3c3ccccc23)c([2H])c([2H])c1-c1c(-c2ccccc2)c2ccccc2c2ccccc12 ; [2H]c1c([2H])c(N(c2cccc3ccccc23)c2cc3ccccc3c3ccccc23)c([2H])c([2H])c1-c1c(-c2ccccc2)c2ccccc2c2ccccc12]

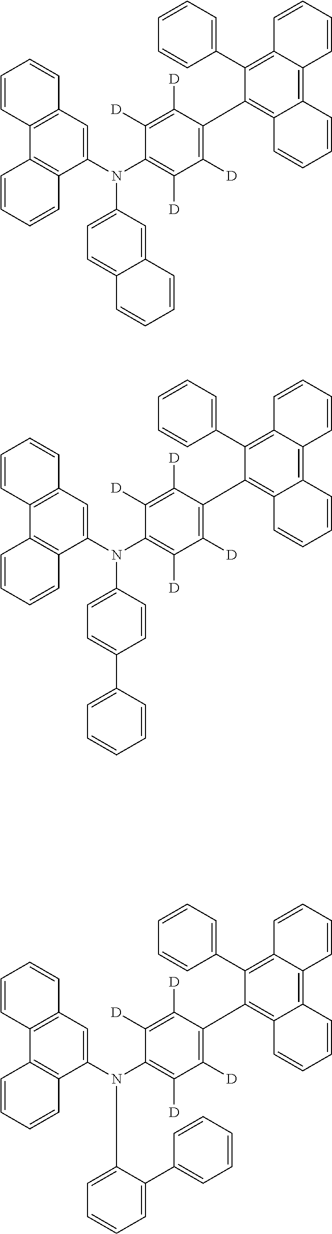 [SMILES: [2H]c1c([2H])c(N(c2ccc3ccccc3c2)c2cc3ccccc3c3ccccc23)c([2H])c([2H])c1-c1c(-c2ccccc2)c2ccccc2c2ccccc12 ; [2H]c1c([2H])c(N(c2ccc(-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccccc2-c2ccccc2)c2cc3ccccc3c3ccccc23)c([2H])c([2H])c1-c1c(-c2ccccc2)c2ccccc2c2ccccc12]

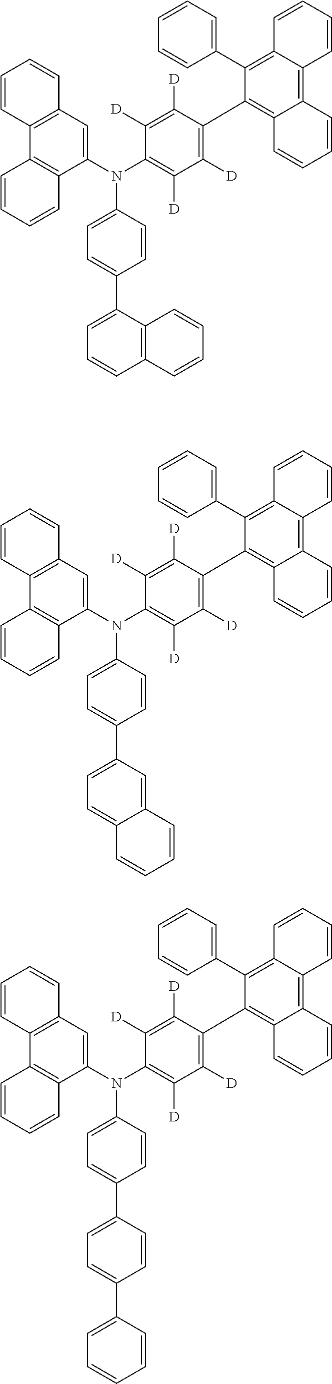 [SMILES: [2H]c1c([2H])c(N(c2ccc(-c3cccc4ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc4ccccc4c3)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1c(-c2ccccc2)c2ccccc2c2ccccc12]

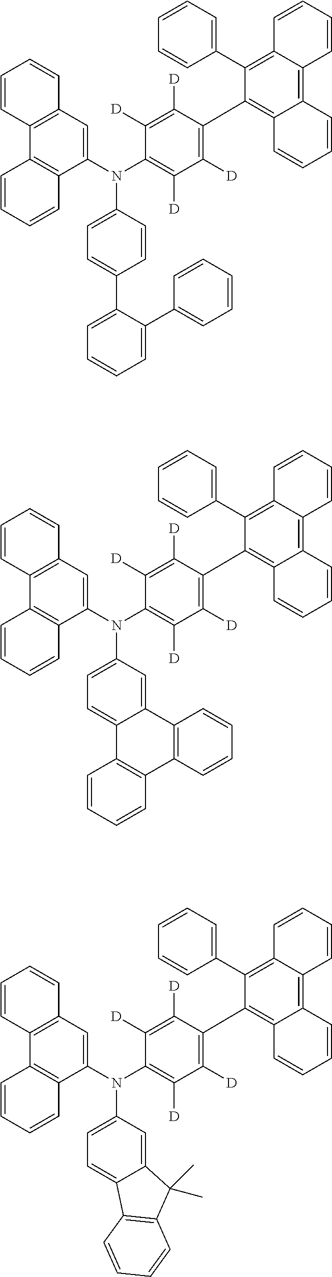 [SMILES: [2H]c1c([2H])c(N(c2ccc(-c3ccccc3-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccc3c4ccccc4c4ccccc4c3c2)c2cc3ccccc3c3ccccc23)c([2H])c([2H])c1-c1c(-c2ccccc2)c2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1c(-c2ccccc2)c2ccccc2c2ccccc12]

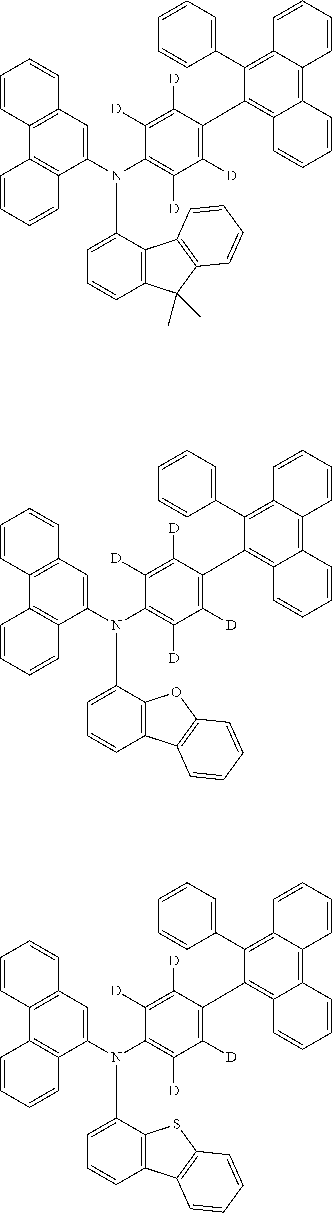 [SMILES: [2H]c1c([2H])c(N(c2cccc3c2-c2ccccc2C3(C)C)c2cc3ccccc3c3ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2oc2ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2sc2ccccc23)c([2H])c([2H])c1-c1c(-c2ccccc2)c2ccccc2c2ccccc12]

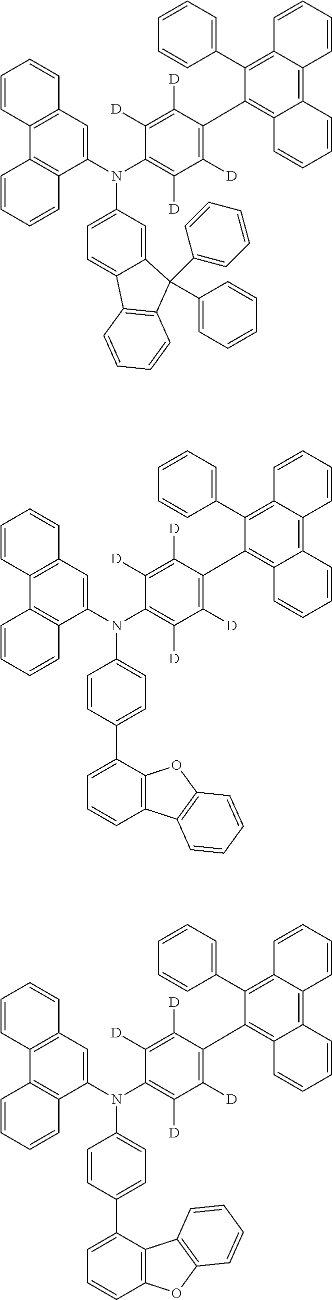 [SMILES: [2H]c1c([2H])c(N(c2ccc3c(c2)C(c2ccccc2)(c2ccccc2)c2ccccc2-3)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1c(-c2ccccc2)c2ccccc2c2ccccc12]

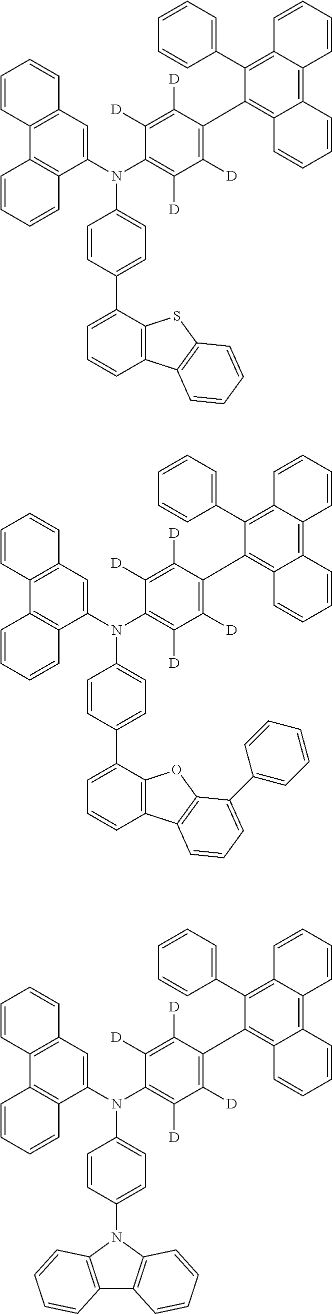 [SMILES: [2H]c1c([2H])c(N(c2ccc(-c3cccc4c3sc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1c(-c2ccccc2)c2ccccc2c2ccccc12]

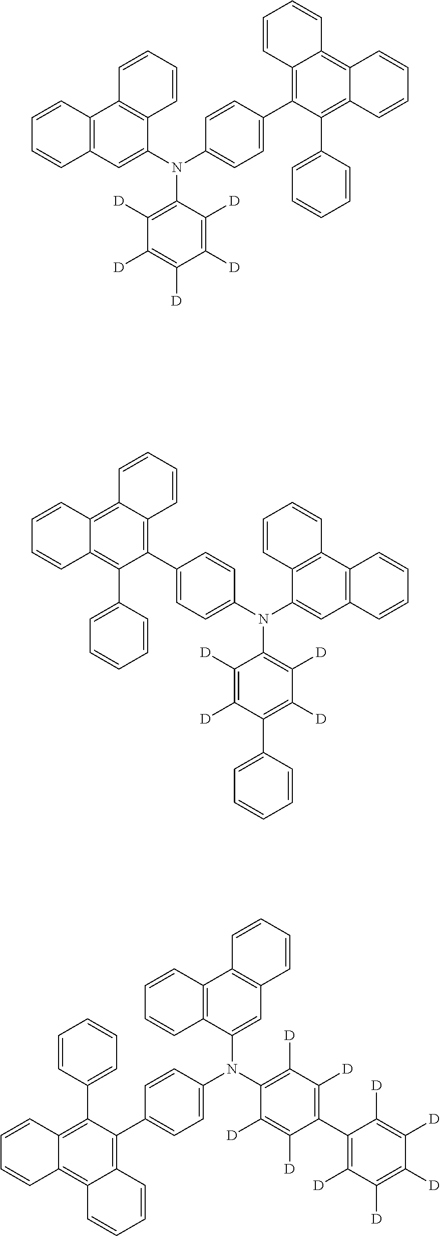 [SMILES: [2H]c1c([2H])c([2H])c(N(c2ccc(-c3c(-c4ccccc4)c4ccccc4c4ccccc34)cc2)c2cc3ccccc3c3ccccc23)c([2H])c1[2H] ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1 ; [2H]c1c([2H])c([2H])c(-c2c([2H])c([2H])c(N(c3ccc(-c4c(-c5ccccc5)c5ccccc5c5ccccc45)cc3)c3cc4ccccc4c4ccccc34)c([2H])c2[2H])c([2H])c1[2H]]

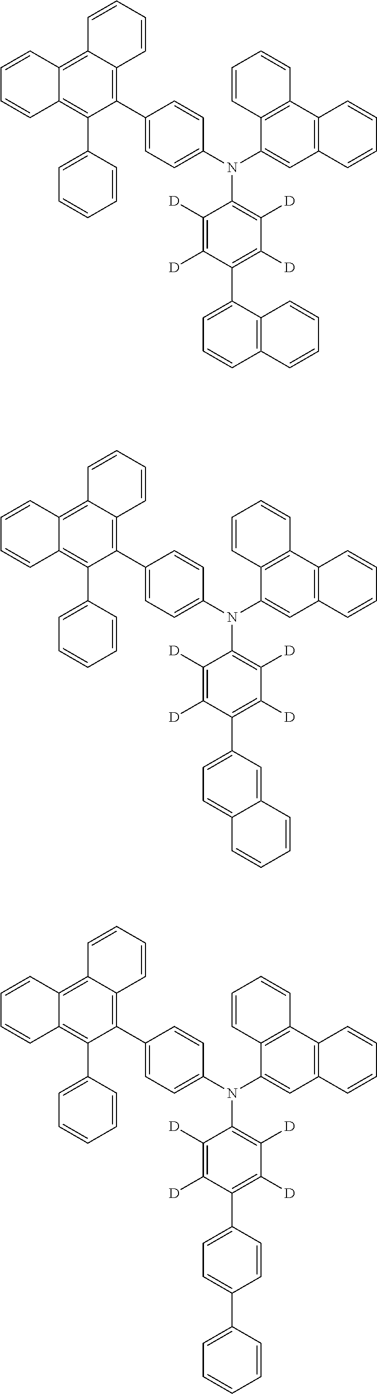 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc2ccccc2c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc(-c2ccccc2)cc1]

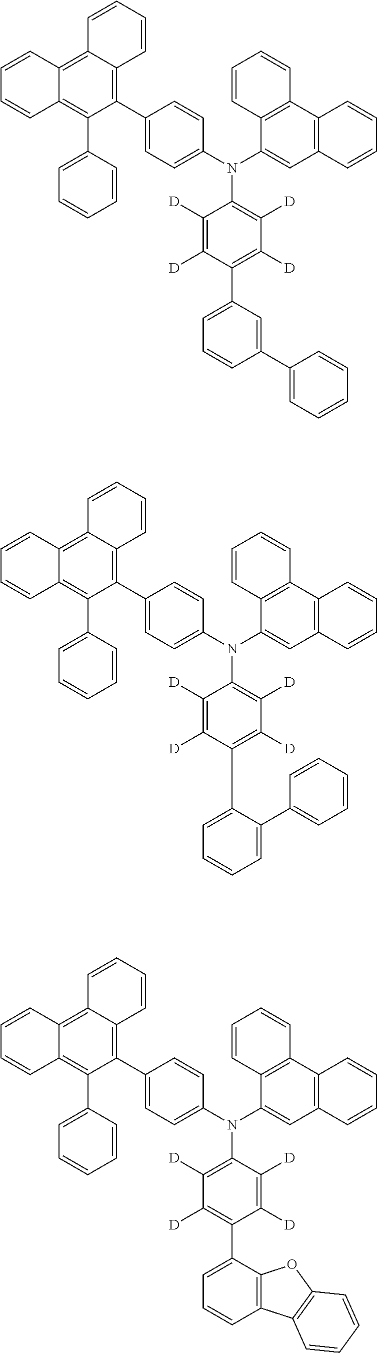 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc(-c2ccccc2)c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1oc1ccccc12]

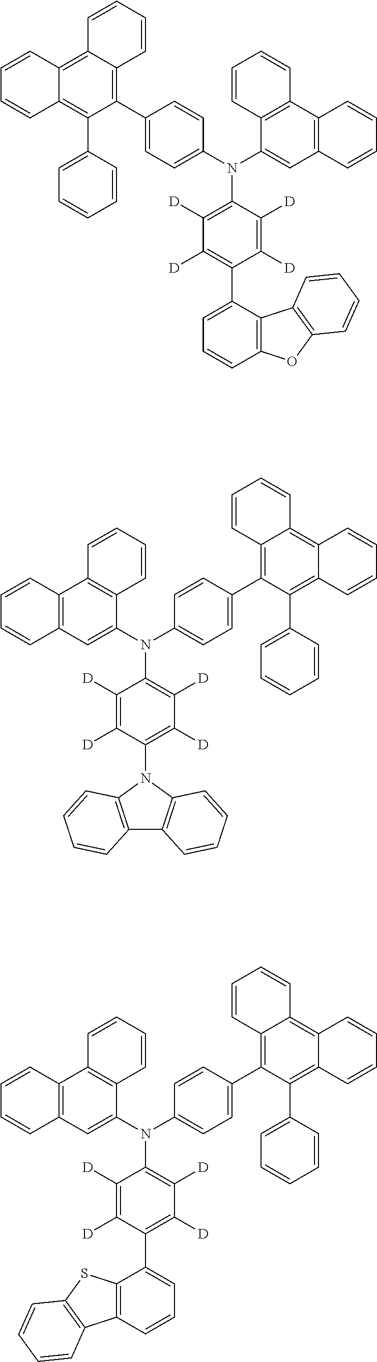 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2oc3ccccc3c12 ; [2H]c1c([2H])c(-n2c3ccccc3c3ccccc32)c([2H])c([2H])c1N(c1ccc(-c2c(-c3ccccc3)c3ccccc3c3ccccc23)cc1)c1cc2ccccc2c2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1sc1ccccc12]

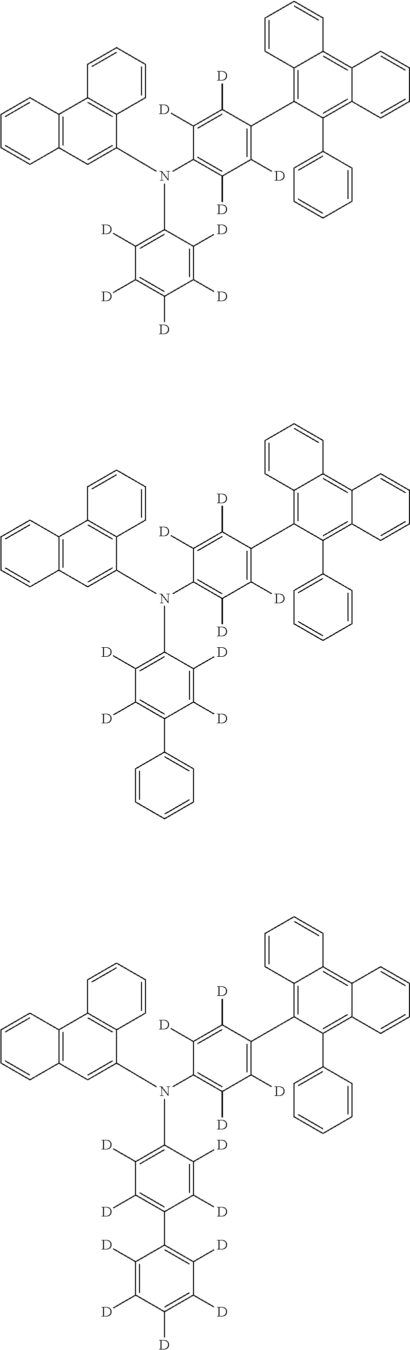 [SMILES: [2H]c1c([2H])c([2H])c(N(c2c([2H])c([2H])c(-c3c(-c4ccccc4)c4ccccc4c4ccccc34)c([2H])c2[2H])c2cc3ccccc3c3ccccc23)c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4c(-c5ccccc5)c5ccccc5c5ccccc45)c([2H])c3[2H])c3cc4ccccc4c4ccccc34)c([2H])c2[2H])c([2H])c1[2H]]

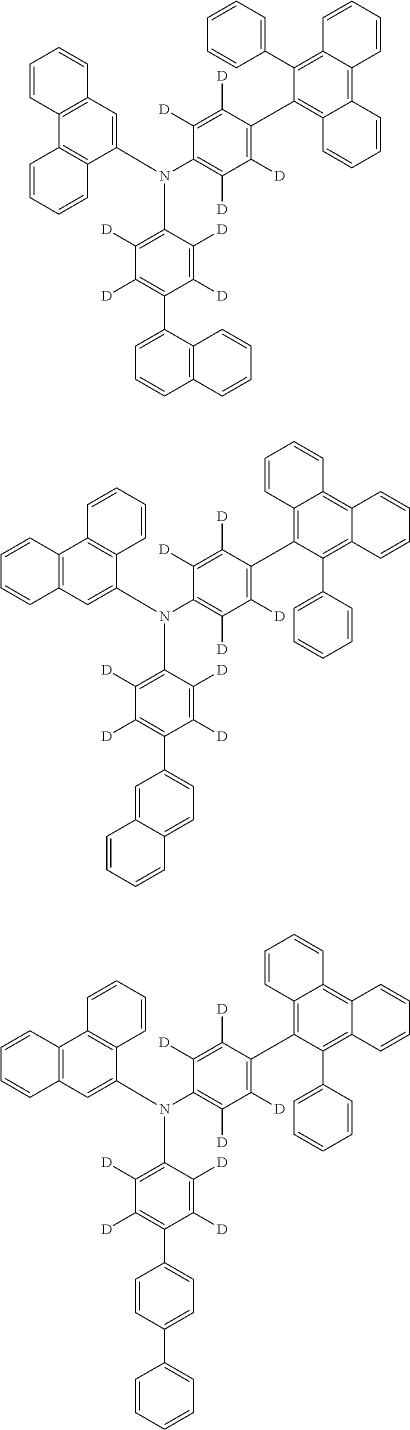 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1cccc2ccccc12 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc(-c2ccccc2)cc1]

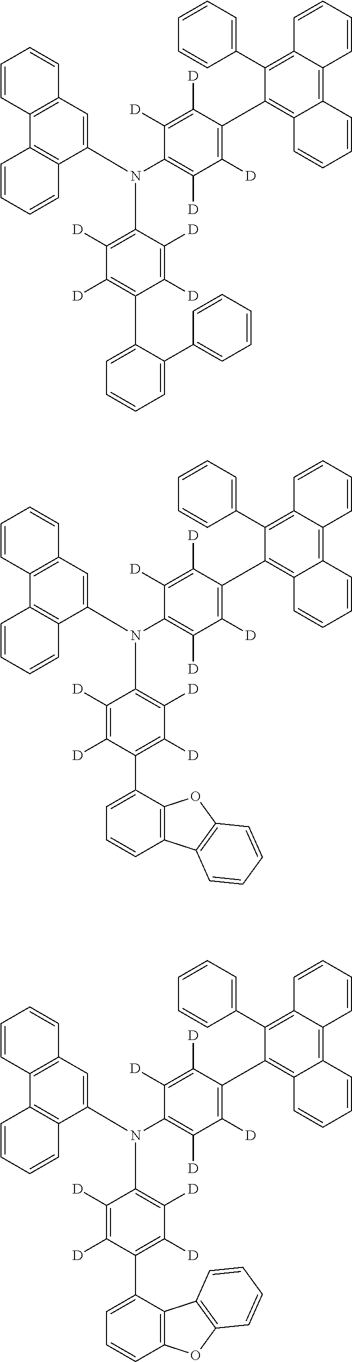 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1-c1ccccc1 ; [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1c(-c2ccccc2)c2ccccc2c2ccccc12]

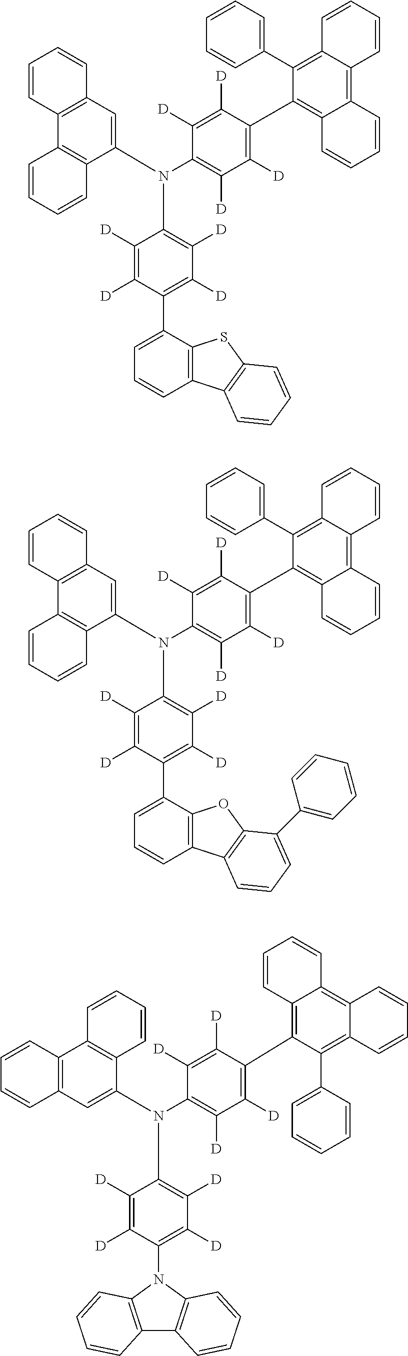 [SMILES: [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1c(-c2ccccc2)c2ccccc2c2ccccc12]

[chemical structure]

[chemical structure]

[chemical structure]

[chemical structure]

[chemical structure]

[chemical structure]

[chemical structure]

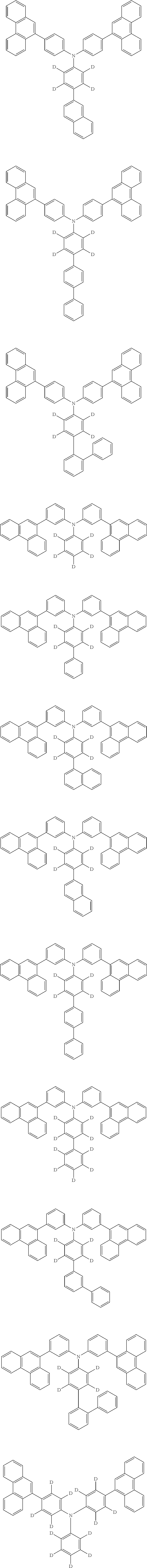

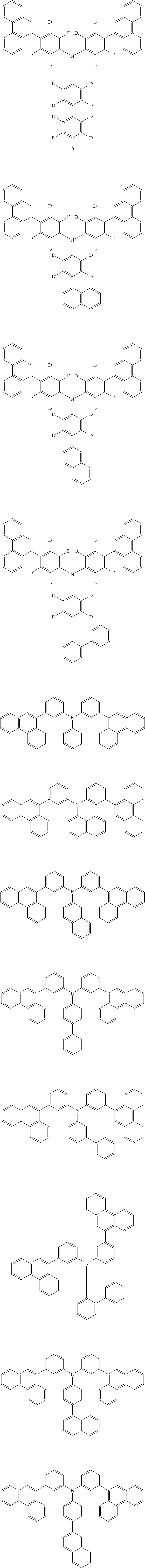

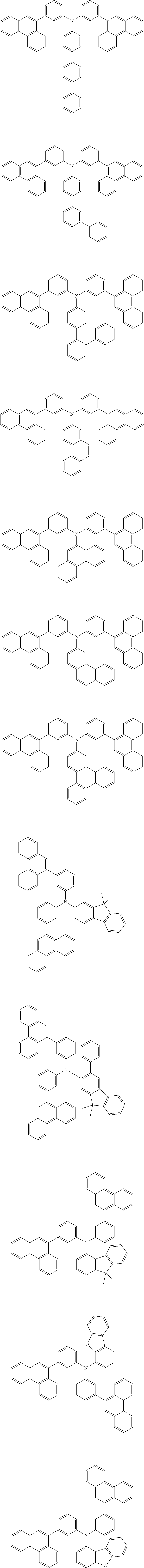

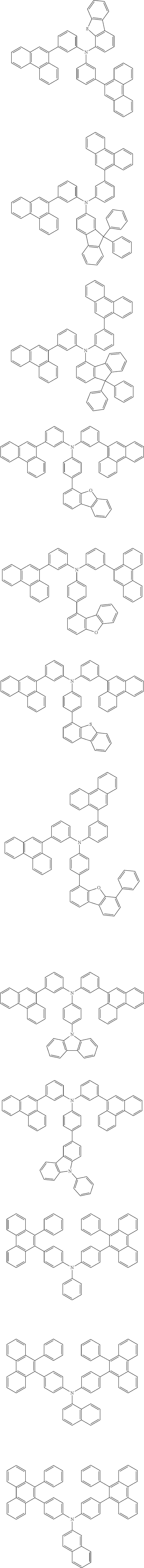

[chemical structure]

[chemical structure]

[chemical structure]

[chemical structure]

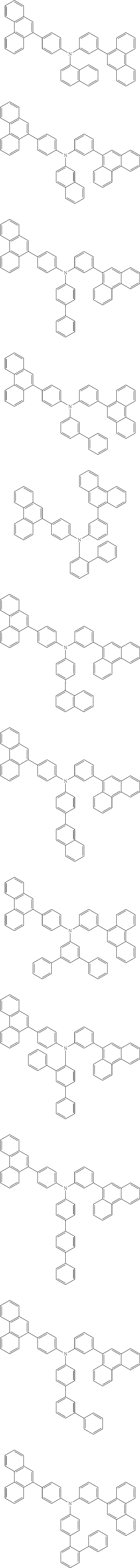

[chemical structure]

[chemical structure]

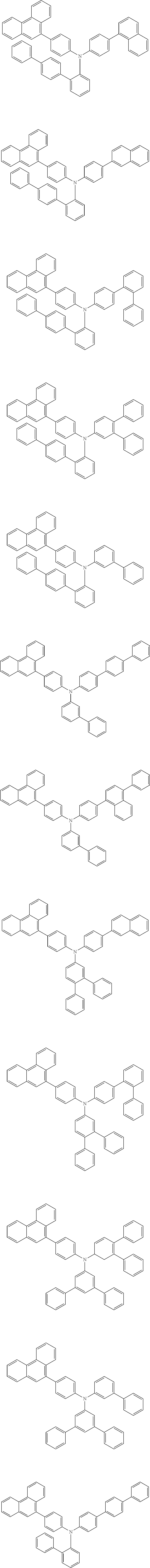

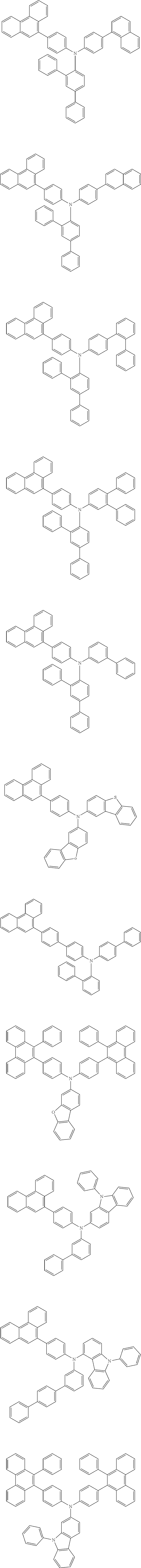

[chemical structure]

[chemical structure]

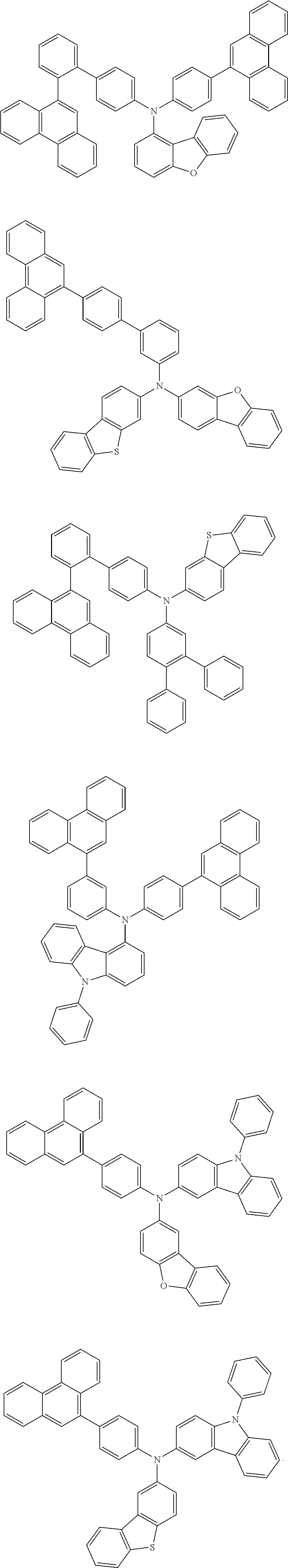

The compound of Chemical Formula 2 can be prepared, for example, according to the preparation method as shown in the following Reaction Scheme 2, an d the other remaining compounds can be prepared in a similar manner.

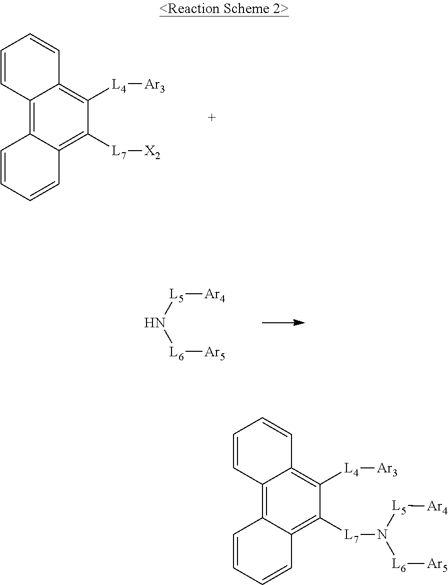

wherein in Reaction Scheme 2, Ar₃ to Ar₅ and L₄ to L₇ are as defined in Chemical Formula 2, and X₂ is halogen, preferably X₂ is chloro or bromo.

The Reaction Scheme 2 is an amine substitution reaction, which is preferably carried out in the presence of a palladium catalyst and a base, and a reactive group for the amine substitution reaction can be modified as known in the art. The above preparation method can be further embodied in the Preparation Examples described hereinafter.

Preferably, the weight ratio of the compound of Chemical Formula 1 and the compound of Chemical Formula 2 in the light emitting layer is 10:90 to 90:10, more preferably 20:80 to 80:20, 30:70 to 70:30 or 40:60 to 60:40.

Meanwhile, the light emitting layer can further include a dopant in addition to the host. The dopant material is not particularly limited as long as it is a material used for the organic light emitting device. As an example, an aromatic amine derivative, a styrylamine compound, a boron complex, a fluoranthene compound, a metal complex, and the like can be mentioned. Specific examples of the aromatic amine derivatives include substituted or unsubstituted fused aromatic ring derivatives having an arylamino group, examples thereof include pyrene, anthracene, chrysene, and periflanthene having the arylamino group, and the like. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

### Hole Blocking Layer

The hole blocking layer is a layer provided between the electron transport layer and the light emitting layer in order to prevent the holes injected in the anode from being transferred to the electron transport layer without being recombined in the light emitting layer, which can also be referred to as a hole inhibition layer or a hole stopping layer. The hole blocking layer is preferably a material having the large ionization energy.

### Electron Transport Layer

The organic light emitting device according to the present disclosure can include an electron transport layer on the light emitting layer, if necessary.

The electron transport layer is a layer that receives the electrons from the electron injection layer formed on the cathode and cathode and transports the electrons to the light emitting layer, and that suppress the transfer of holes from the light emitting layer, and an electron transport material is suitably a material which can receive electrons well from a cathode and transfer the electrons to a light emitting layer, and has a large mobility for electrons.

Specific examples of the electron transport material include: an Al complex of 8-hydroxyquinoline, a complex including Alq₃, an organic radical compound, a hydroxyflavone-metal complex, and the like, but are not limited thereto. The electron transport layer can be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

### Electron Injection Layer

The organic light emitting device according to the present disclosure can further include an electron injection layer on the light emitting layer (or on an electron transport layer when the electron transport layer is present), if necessary.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the materials that can be used as the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)-gallium, bis(2-methyl-8-quinolinato)(1-naphtholato)aluminum, bis(2-methyl-8-quinolinato)(2-naphtholato)gallium, and the like, but are not limited thereto.

Meanwhile, in the present disclosure, the “electron injection and transport layer” is a layer that performs both the roles of the electron injection layer and the electron transport layer, and the material that serves as each layer can be used alone or in combination, but is not limited thereto.

### Organic Light Emitting Device

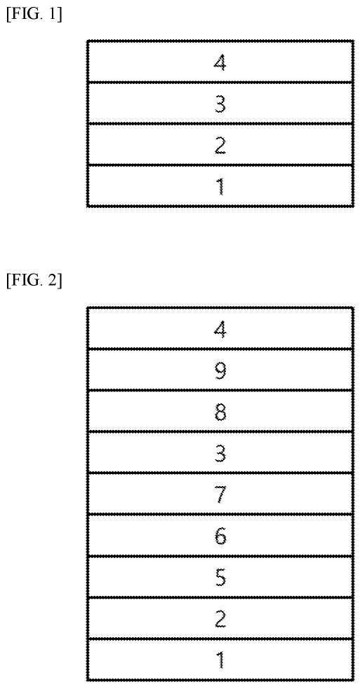

The structure of the organic light emitting device according to the present disclosure is illustrated in FIG. 1. FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

The organic light emitting device according to the present disclosure can be manufactured by sequentially stacking the above-described structures. In this case, the organic light emitting device can be manufactured by depositing a metal, metal oxides having conductivity, or an alloy thereof on the substrate by using a PVD (physical vapor deposition) method such as a sputtering method or an e-beam evaporation method to form the anode, forming the respective layers described above thereon, and then depositing a material that can be used as the cathode thereon. In addition to such a method, the organic light emitting device can be manufactured by sequentially depositing from the cathode material to the anode material on a substrate in the reverse order of the above-mentioned configuration (WO 2003/012890). Further, the light emitting layer can be formed by subjecting hosts and dopants to a vacuum deposition method and a solution coating method. Herein, the solution coating method means a spin coating, a dip coating, a doctor blading, an inkjet printing, a screen printing, a spray method, a roll coating, or the like, but is not limited thereto.

On the other hand, the organic light emitting device according to the present disclosure can be a front side emission type, a back side emission type, or a double side emission type according to the used material.

Hereinafter, preferred examples of the present disclosure are presented to aid in the understanding of the invention. However, these examples are presented for illustrative purposes only, and the scope of the present disclosure is not limited thereto.

### PREPARATION EXAMPLE

### Preparation Example 1-1: Preparation of Compound 1-1

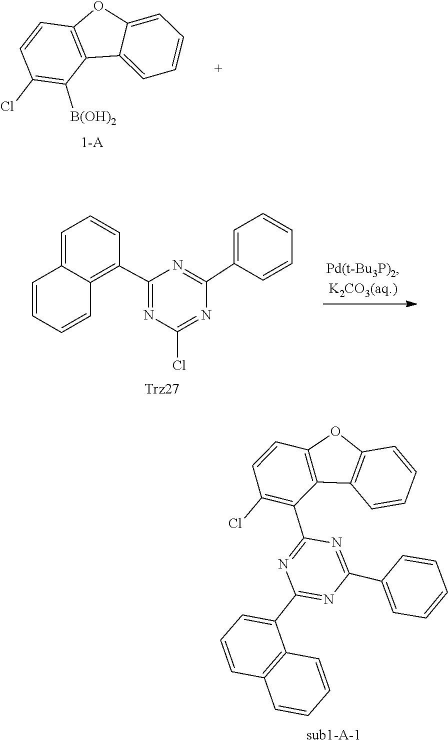 [SMILES: OB(O)c1c(Cl)ccc2oc3ccccc3c12 ; Clc1nc(-c2ccccc2)nc(-c2cccc3ccccc23)n1 ; Clc1ccc2oc3ccccc3c2c1-c1nc(-c2ccccc2)nc(-c2cccc3ccccc23)n1]

Compound 1-A (15 g, 60.9 mmol) and Compound Trz27 (19.3 g, 60.9 mmol) were added to 300 ml of THF under a nitrogen atmosphere, and the mixture was stirred and refluxed. Then, potassium carbonate (16.8 g, 121.7 mmol) was dissolved in 50 ml of water, added thereto, and the mixture was sufficiently stirred and then bis(tri-tert-butylphosphine)palladium (0) (0.3 g, 0.6 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 20.9 g of Compound sub1-A-1. (Yield: 71%, MS: [M+H]⁺=484)

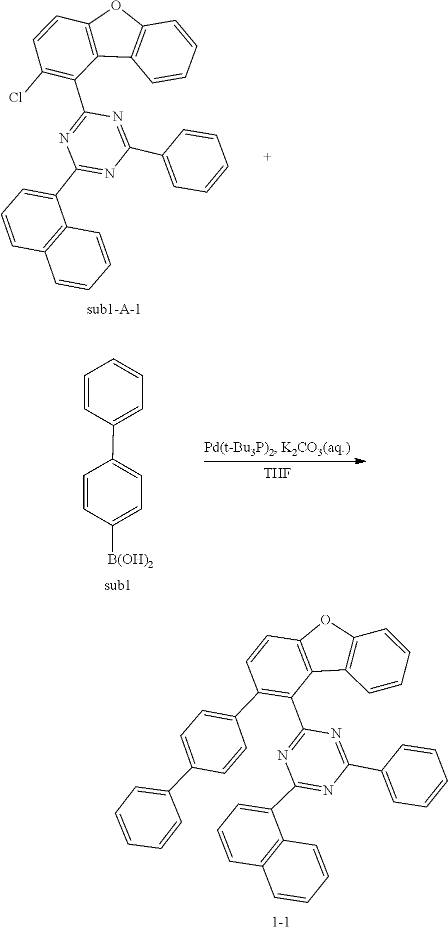 [SMILES: Clc1ccc2oc3ccccc3c2c1-c1nc(-c2ccccc2)nc(-c2cccc3ccccc23)n1 ; OB(O)c1ccc(-c2ccccc2)cc1 ; c1ccc(-c2ccc(-c3ccc4oc5ccccc5c4c3-c3nc(-c4ccccc4)nc(-c4cccc5ccccc45)n3)cc2)cc1 ; "THF"]

Compound sub1-A-1 (15 g, 31 mmol) and Compound sub1 (6.1 g, 31 mmol) were added to 300 ml of THF under a nitrogen atmosphere, and the mixture was stirred and refluxed. Then, potassium carbonate (8.6 g, 62 mmol) was dissolved in 26 ml of water, added thereto, and the mixture was sufficiently stirred and then bis(tri-tert-butylphosphine)palladium (0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.3 g of Compound 1-1. (Yield: 66%, MS: [M+H]⁺=602)

### Preparation Example 1-2: Preparation of Compound 1-2

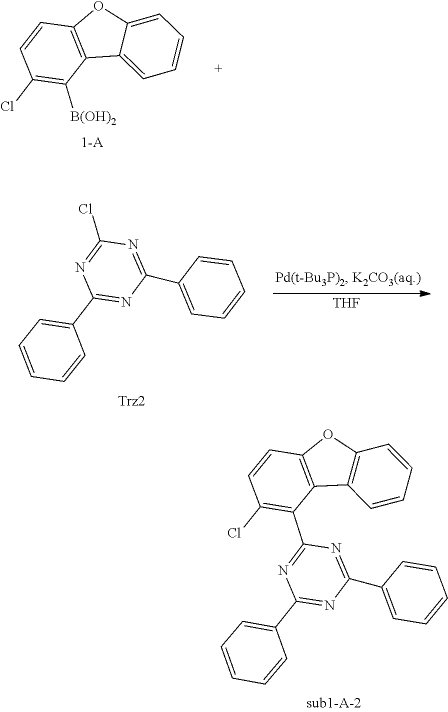 [SMILES: OB(O)c1c(Cl)ccc2oc3ccccc3c12 ; Clc1nc(-c2ccccc2)nc(-c2ccccc2)n1 ; Clc1ccc2oc3ccccc3c2c1-c1nc(-c2ccccc2)nc(-c2ccccc2)n1 ; "THF"]

Compound 1-A (15 g, 60.9 mmol) and Compound Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium (0) (0.3 g, 0.6 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 19.5 g of Compound sub1-A-2. (Yield: 74%, MS: [M+H]⁺=434)

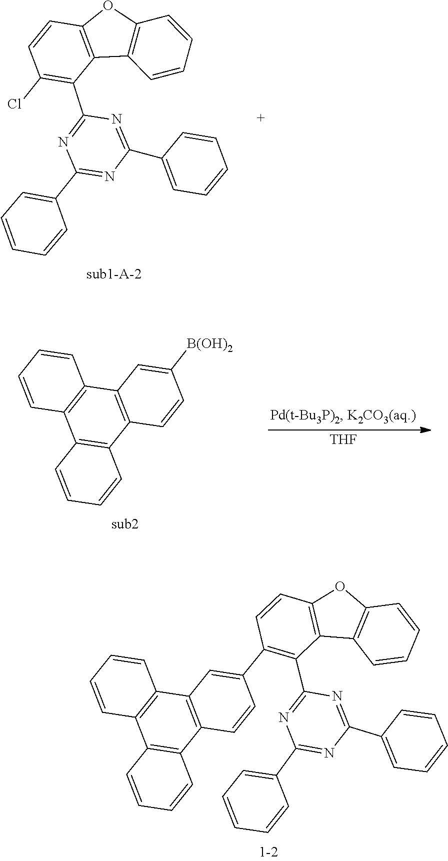 [SMILES: Clc1ccc2oc3ccccc3c2c1-c1nc(-c2ccccc2)nc(-c2ccccc2)n1 ; OB(O)c1ccc2c3ccccc3c3ccccc3c2c1 ; c1ccc(-c2nc(-c3ccccc3)nc(-c3c(-c4ccc5c6ccccc6c6ccccc6c5c4)ccc4oc5ccccc5c34)n2)cc1 ; "THF"]

Compound sub1-A-2 (15 g, 34.6 mmol) and Compound sub2 (9.4 g, 34.6 mmol) were added to 300 ml of THF under a nitrogen atmosphere, and the mixture was stirred and refluxed. Then, potassium carbonate (9.6 g, 69.1 mmol) was dissolved in 29 ml of water, added thereto, and the mixture was sufficiently stirred and then bis(tri-tert-butylphosphine)palladium(0) (0.2 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.3 g of Compound 1-2. (Yield: 66%, MS: [M+H]⁺=626)

### Preparation Example 1-3: Preparation of Compound 1-3

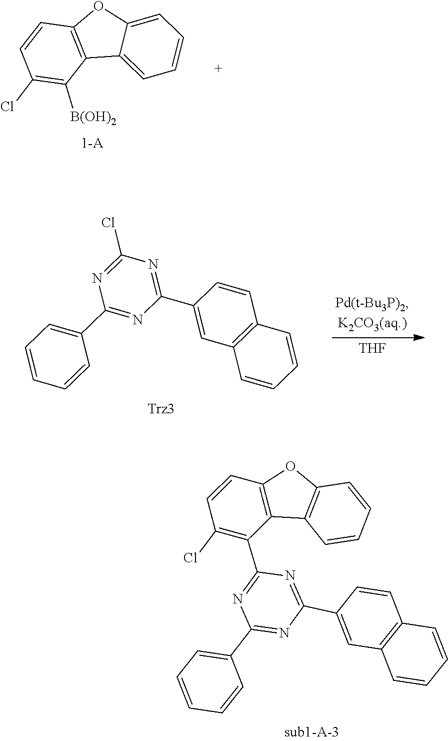 [SMILES: OB(O)c1c(Cl)ccc2oc3ccccc3c12 ; Clc1nc(-c2ccccc2)nc(-c2ccc3ccccc3c2)n1 ; Clc1ccc2oc3ccccc3c2c1-c1nc(-c2ccccc2)nc(-c2ccc3ccccc3c2)n1 ; "THF"]

Compound 1-A (15 g, 60.9 mmol) and Compound Trz3 (19.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium (0) (0.3 g, 0.6 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 23.2 g of Compound sub1-A-3. (Yield: 79%, MS: [M+H]⁺=484)

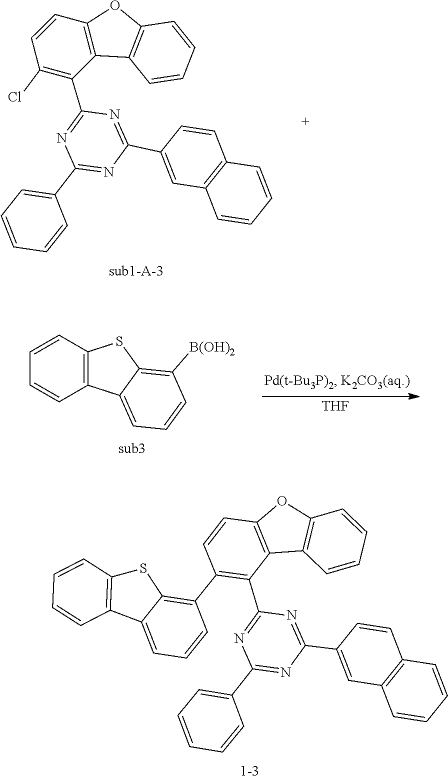 [SMILES: Clc1ccc2oc3ccccc3c2c1-c1nc(-c2ccccc2)nc(-c2ccc3ccccc3c2)n1 ; OB(O)c1cccc2c1sc1ccccc12 ; c1ccc(-c2nc(-c3ccc4ccccc4c3)nc(-c3c(-c4cccc5c4sc4ccccc45)ccc4oc5ccccc5c34)n2)cc1 ; "THF"]

Compound sub1-A-3 (15 g, 31 mmol) and Compound sub3 (7.1 g, 31 mmol) were added to 300 ml of THF under a nitrogen atmosphere, and the mixture was stirred and refluxed. Then, potassium carbonate (8.6 g, 62 mmol) was dissolved in 26 ml of water, added thereto, and the mixture was sufficiently stirred and then bis(tri-tert-butylphosphine)palladium(0) (0.2 g, 0.3 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.9 g of Compound 1-3. (Yield: 66%, MS: [M+H]⁺=632)

### Preparation Example 1-4: Preparation of Compound 1-4

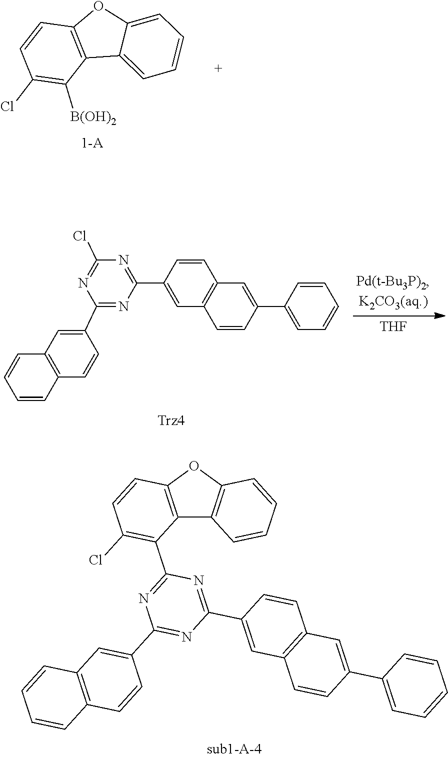 [SMILES: OB(O)c1c(Cl)ccc2oc3ccccc3c12 ; Clc1nc(-c2ccc3ccccc3c2)nc(-c2ccc3cc(-c4ccccc4)ccc3c2)n1 ; Clc1ccc2oc3ccccc3c2c1-c1nc(-c2ccc3ccccc3c2)nc(-c2ccc3cc(-c4ccccc4)ccc3c2)n1 ; "THF"]

Compound 1-A (15 g, 60.9 mmol) and Compound Trz4 (27 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium (0) (0.3 g, 0.6 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 26 g of Compound sub1-A-4. (Yield: 70%, MS: [M+H]⁺=610)

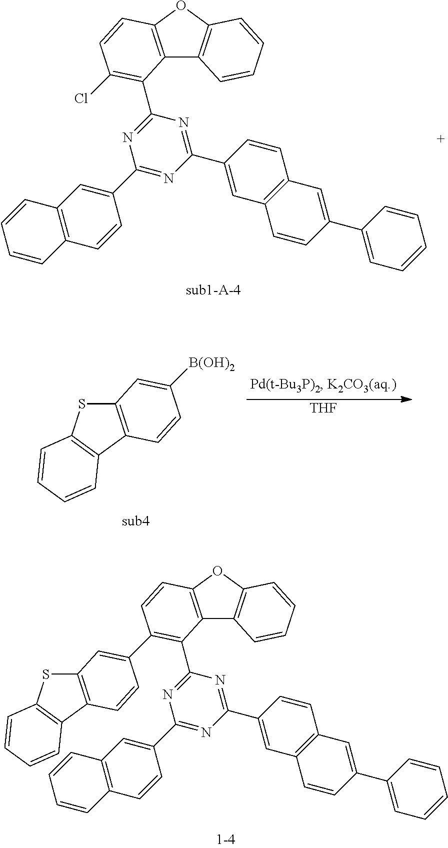 [SMILES: Clc1ccc2oc3ccccc3c2c1-c1nc(-c2ccc3ccccc3c2)nc(-c2ccc3cc(-c4ccccc4)ccc3c2)n1 ; OB(O)c1ccc2c(c1)sc1ccccc12 ; c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1 ; "THF"]

Compound sub1-A-4 (15 g, 24.6 mmol) and Compound sub4 (5.6 g, 24.6 mmol) were added to 300 ml of THF under a nitrogen atmosphere, and the mixture was stirred and refluxed. Then potassium carbonate (6.8 g, 49.2 mmol) was dissolved in 20 ml of water, added thereto, and the mixture was sufficiently stirred and then bis(tri-tert-butylphosphine)palladium(0) (0.1 g, 0.2 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 11.2 g of Compound 1-4. (Yield: 60%, MS: [M+H]⁺=758)

### Preparation Example 1-5: Preparation of Compound 1-5

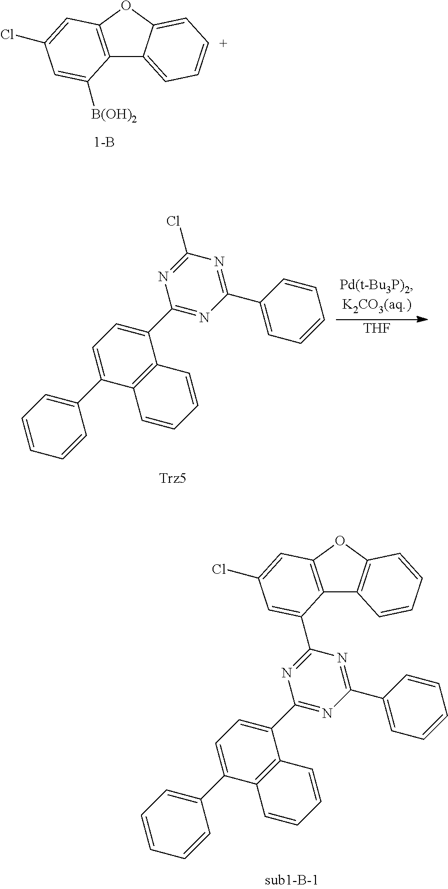 [SMILES: OB(O)c1cc(Cl)cc2oc3c(c12)=CC=[C+]C=3 ; Clc1nc(-c2ccccc2)nc(-c2ccc(-c3ccccc3)c3ccccc23)n1 ; Clc1cc(-c2nc(-c3ccccc3)nc(-c3ccc(-c4ccccc4)c4ccccc34)n2)c2c(c1)oc1ccccc12 ; "THF"]

Compound 1-B (15 g, 60.9 mmol) and Compound Trz5 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium (0) (0.3 g, 0.6 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 26.2 g of Compound sub1-B-1. (Yield: 77%, MS: [M+H]⁺=560)

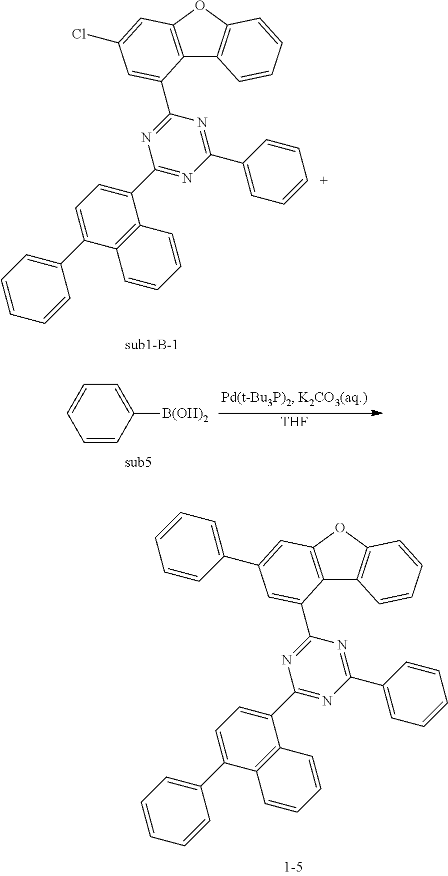 [SMILES: Clc1cc(-c2nc(-c3ccccc3)nc(-c3ccc(-c4ccccc4)c4ccccc34)n2)c2c(c1)oc1ccccc12 ; OB(O)c1ccccc1 ; c1ccc(-c2cc(-c3nc(-c4ccccc4)nc(-c4ccc(-c5ccccc5)c5ccccc45)n3)c3c(c2)oc2ccccc23)cc1 ; "THF"]

Compound sub1-B-1 (15 g, 26.8 mmol) and Compound sub5 (3.3 g, 26.8 mmol) were added to 300 ml of THF under a nitrogen atmosphere, and the mixture was stirred and refluxed. Then, potassium carbonate (7.4 g, 53.6 mmol) was dissolved in 22 ml of water, added thereto, and the mixture was sufficiently stirred and then bis(tri-tert-butylphosphine)palladium (0) (0.1 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.9 g of Compound 1-5. (Yield: 80%, MS: [M+H]⁺=602)

### Preparation Example 1-6: Preparation of Compound 1-6

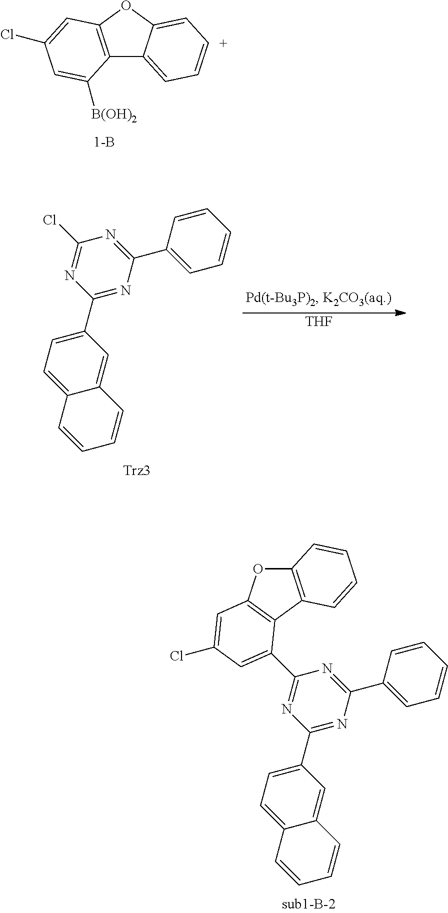 [SMILES: OB(O)c1cc(Cl)cc2oc3c(c12)=CC=[C+]C=3 ; Clc1nc(-c2ccccc2)nc(-c2ccc3ccccc3c2)n1 ; Clc1cc(-c2nc(-c3ccccc3)nc(-c3ccc4ccccc4c3)n2)c2c(c1)oc1ccccc12 ; "THF"]

Compound 1-B (15 g, 60.9 mmol) and Compound Trz3 (19.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 18.2 g of Compound sub1-B-2. (Yield: 62%, MS: [M+H]⁺=484)

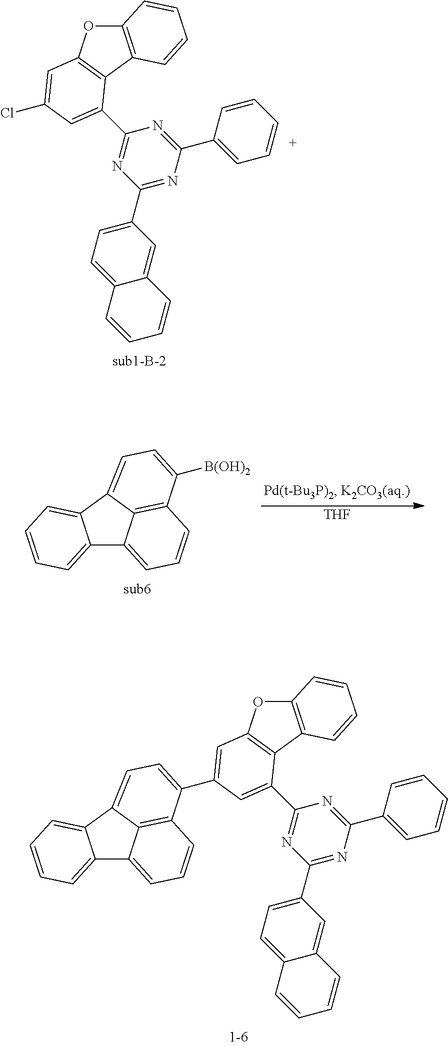 [SMILES: Clc1cc(-c2nc(-c3ccccc3)nc(-c3ccc4ccccc4c3)n2)c2c(c1)oc1ccccc12 ; OB(O)c1ccc2c3c(cccc13)-c1ccccc1-2 ; c1ccc(-c2nc(-c3ccc4ccccc4c3)nc(-c3cc(-c4ccc5c6c(cccc46)-c4ccccc4-5)cc4oc5ccccc5c34)n2)cc1 ; "THF"]

Compound sub1-B-2 (15 g, 31 mmol) and Compound sub6 (7.6 g, 31 mmol) were added to 300 ml of THF under a nitrogen atmosphere, and the mixture was stirred and refluxed. Then, potassium carbonate (8.6 g, 62 mmol) was dissolved in 26 ml of water, added thereto, and the mixture was sufficiently stirred and then bis(tri-tert-butylphosphine)palladium(0) (0.2 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15.3 g of Compound 1-6. (Yield: 76%, MS: [M+H]⁺=650)

### Preparation Example 1-7: Preparation of Compound 1-7

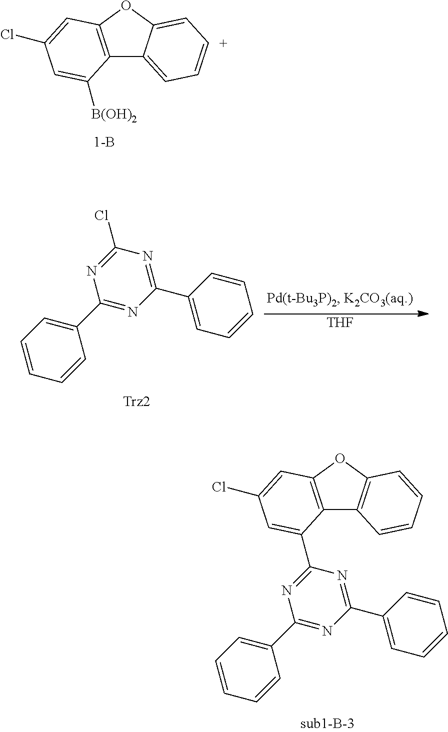 [SMILES: OB(O)c1cc(Cl)cc2oc3c(c12)=CC=[C+]C=3 ; Clc1nc(-c2ccccc2)nc(-c2ccccc2)n1 ; Clc1cc(-c2nc(-c3ccccc3)nc(-c3ccccc3)n2)c2c(c1)oc1ccccc12 ; "THF"]

Compound 1-B (15 g, 60.9 mmol) and Compound Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 20.8 g of Compound sub1-B-3. (Yield: 79%, MS: [M+H]⁺=434)

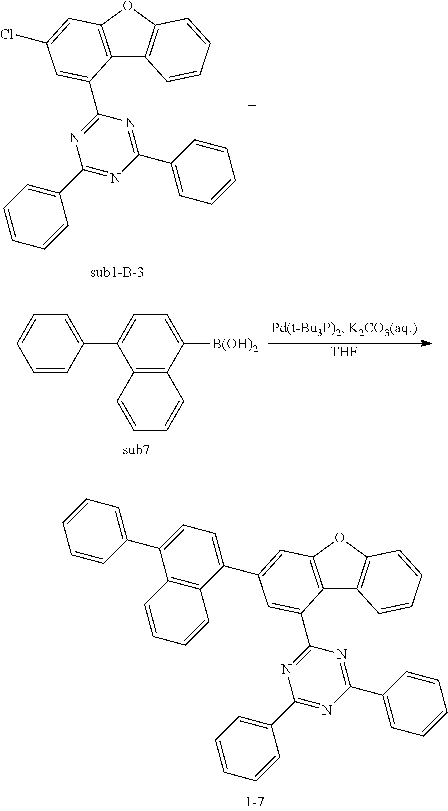 [SMILES: Clc1cc(-c2nc(-c3ccccc3)nc(-c3ccccc3)n2)c2c(c1)oc1ccccc12 ; OB(O)c1ccc(-c2ccccc2)c2ccccc12 ; c1ccc(-c2nc(-c3ccccc3)nc(-c3cc(-c4ccc(-c5ccccc5)c5ccccc45)cc4oc5ccccc5c34)n2)cc1 ; "THF"]

Compound sub1-B-3 (15 g, 34.6 mmol) and Compound sub7 (8.6 g, 34.6 mmol) were added to 300 ml of THF under a nitrogen atmosphere, and the mixture was stirred and refluxed. Then, potassium carbonate (9.6 g, 69.1 mmol) was dissolved in 29 ml of water, added thereto, and the mixture was sufficiently stirred and then bis(tri-tert-butylphosphine)palladium(0) (0.2 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15.4 g of Compound 1-7. (Yield: 74%, MS: [M+H]⁺=602)

### Preparation Example 1-8: Preparation of Compound 1-8

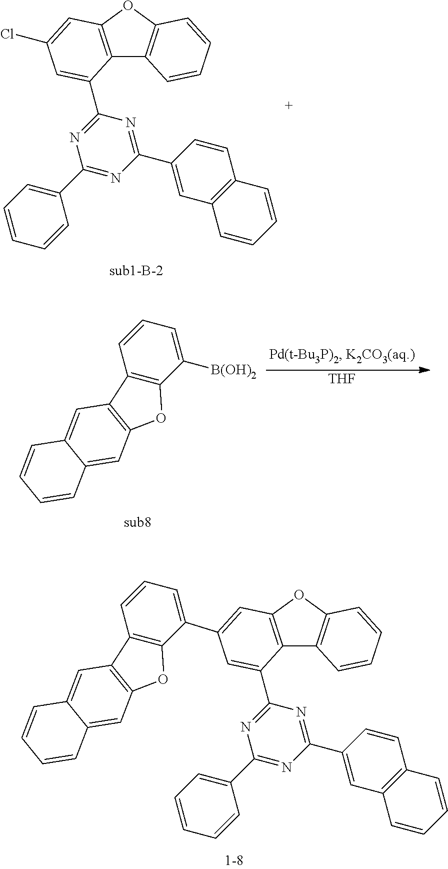 [SMILES: Clc1cc(-c2nc(-c3ccccc3)nc(-c3ccc4ccccc4c3)n2)c2c(c1)oc1ccccc12 ; OB(O)c1cccc2c1oc1cc3ccccc3cc12 ; c1ccc(-c2nc(-c3ccc4ccccc4c3)nc(-c3cc(-c4cccc5c4oc4cc6ccccc6cc45)cc4oc5ccccc5c34)n2)cc1 ; "THF"]

Compound sub1-B-2 (15 g, 31 mmol) and Compound sub8 (8.1 g, 31 mmol) were added to 300 ml of THF under a nitrogen atmosphere, and the mixture was stirred and refluxed. Then, potassium carbonate (8.6 g, 62 mmol) was dissolved in 26 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15.5 g of Compound 1-8. (Yield: 75%, MS: [M+H]⁺=666)

### Preparation Example 1-9: Preparation of Compound 1-9

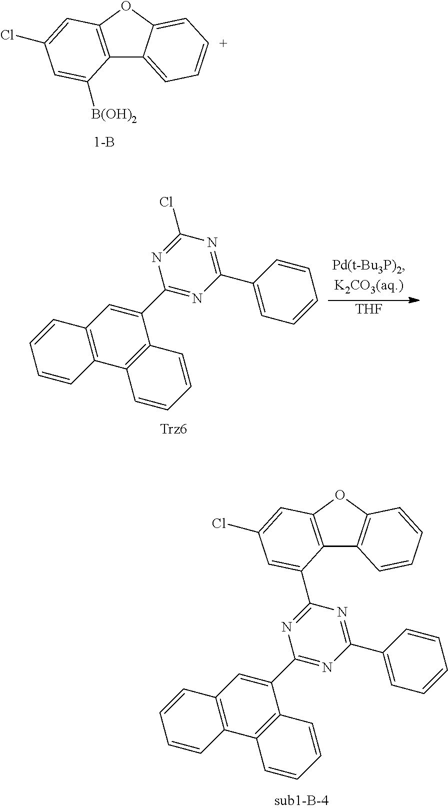 [SMILES: OB(O)c1cc(Cl)cc2oc3c(c12)=CC=[C+]C=3 ; Clc1nc(-c2ccccc2)nc(-c2cc3ccccc3c3ccccc23)n1 ; Clc1cc(-c2nc(-c3ccccc3)nc(-c3cc4ccccc4c4ccccc34)n2)c2c(c1)oc1ccccc12 ; "THF"]

Compound 1-B (15 g, 60.9 mmol) and Compound Trz6 (22.4 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 23.7 g of Compound sub1-B-4. (Yield: 73%, MS: [M+H]⁺=534)

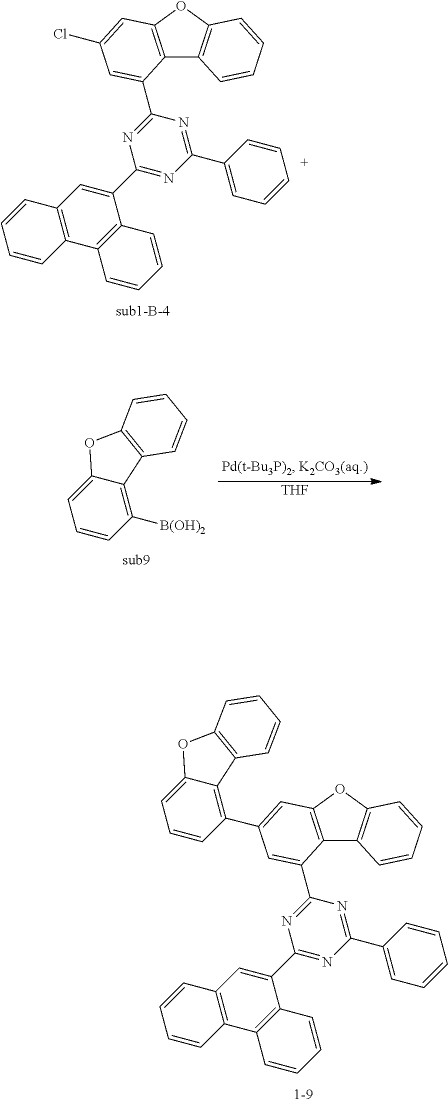 [SMILES: Clc1cc(-c2nc(-c3ccccc3)nc(-c3cc4ccccc4c4ccccc34)n2)c2c(c1)oc1ccccc12 ; OB(O)c1cccc2oc3ccccc3c12 ; c1ccc(-c2nc(-c3cc4ccccc4c4ccccc34)nc(-c3cc(-c4cccc5oc6ccccc6c45)cc4oc5ccccc5c34)n2)cc1 ; "THF"]

Compound sub1-B-4 (15 g, 28.1 mmol) and Compound sub9 (6 g, 28.1 mmol) were added to 300 ml of THF under a nitrogen atmosphere, and the mixture was stirred and refluxed. Then, potassium carbonate (7.8 g, 56.2 mmol) was dissolved in 23 ml of water, added thereto, and the mixture was sufficiently stirred and then bis(tri-tert-butylphosphine)palladium(0) (0.1 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 11.6 g of Compound 1-9. (Yield: 62%, MS: [M+H]⁺=666)

### Preparation Example 1-10: Preparation of Compound 1-10

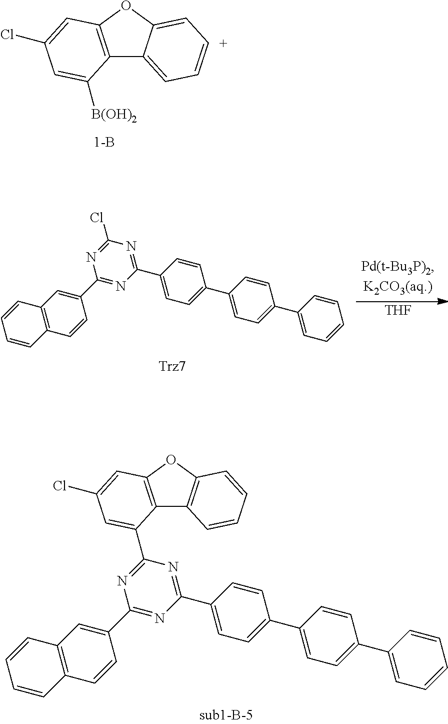 [SMILES: OB(O)c1cc(Cl)cc2oc3c(c12)=CC=[C+]C=3 ; Clc1nc(-c2ccc(-c3ccc(-c4ccccc4)cc3)cc2)nc(-c2ccc3ccccc3c2)n1 ; Clc1cc(-c2nc(-c3ccc(-c4ccc(-c5ccccc5)cc4)cc3)nc(-c3ccc4ccccc4c3)n2)c2c(c1)oc1ccccc12 ; "THF"]

Compound 1-B (15 g, 60.9 mmol) and Compound Trz7 (28.6 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 28.6 g of Compound sub1-B-5. (Yield: 74%, MS: [M+H]⁺=636)

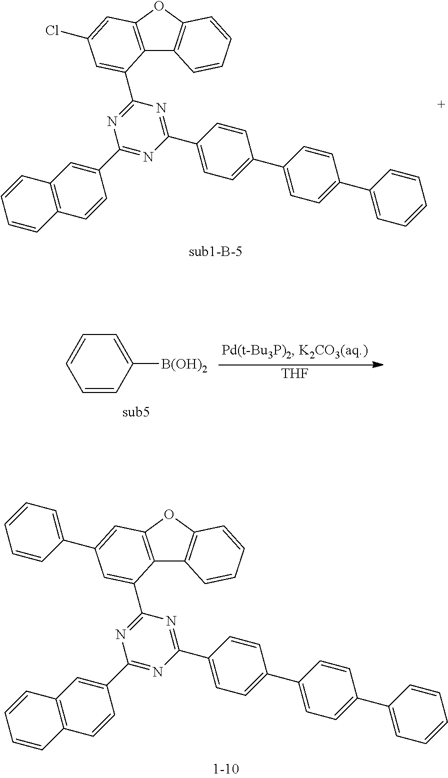 [SMILES: Clc1cc(-c2nc(-c3ccc(-c4ccc(-c5ccccc5)cc4)cc3)nc(-c3ccc4ccccc4c3)n2)c2c(c1)oc1ccccc12 ; OB(O)c1ccccc1 ; c1ccc(-c2ccc(-c3ccc(-c4nc(-c5ccc6ccccc6c5)nc(-c5cc(-c6ccccc6)cc6oc7ccccc7c56)n4)cc3)cc2)cc1 ; "THF"]

Compound sub1-B-5 (15 g, 23.6 mmol) and Compound sub5 (2.9 g, 23.6 mmol) were added to 300 ml of THF under a nitrogen atmosphere, and the mixture was stirred and refluxed. Then, potassium carbonate (6.5 g, 47.2 mmol) was dissolved in 20 ml of water, added thereto, and the mixture was sufficiently stirred and then bis(tri-tert-butylphosphine)palladium(0) (0.1 g, 0.2 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.4 g of Compound 1-10. (Yield: 65%, MS: [M+H]⁺=678)

### Preparation Example 1-11: Preparation of Compound 1-11

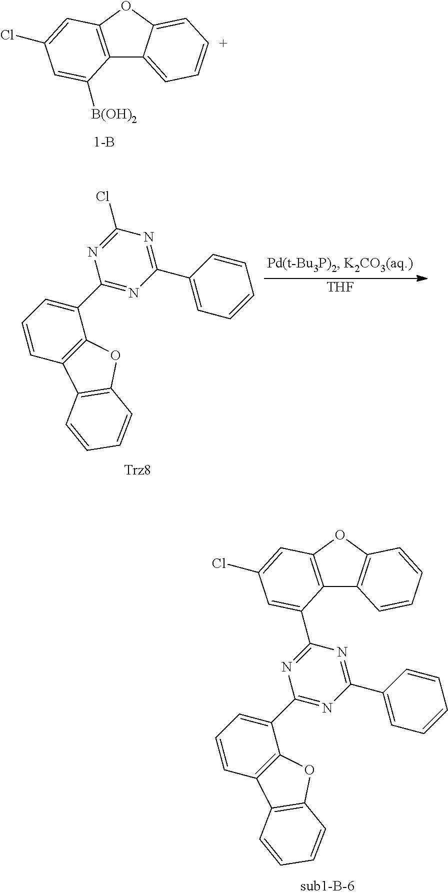 [SMILES: OB(O)c1cc(Cl)cc2oc3c(c12)=CC=[C+]C=3 ; Clc1nc(-c2ccccc2)nc(-c2cccc3c2oc2ccccc23)n1 ; Clc1cc(-c2nc(-c3ccccc3)nc(-c3cccc4c3oc3ccccc34)n2)c2c(c1)oc1ccccc12 ; "THF"]

Compound 1-B (15 g, 60.9 mmol) and Compound Trz8 (21.8 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 20.1 g of Compound sub1-B-6. (Yield: 63%, MS: [M+H]⁺=524)

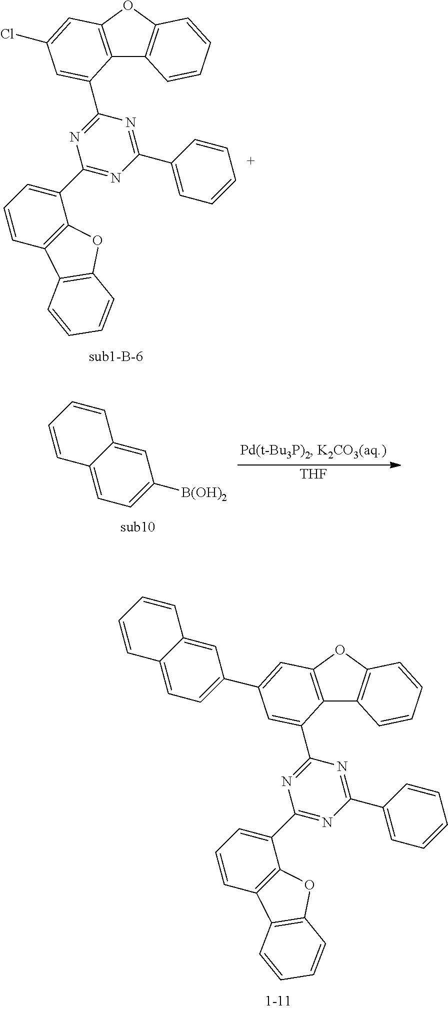 [SMILES: Clc1cc(-c2nc(-c3ccccc3)nc(-c3cccc4c3oc3ccccc34)n2)c2c(c1)oc1ccccc12 ; OB(O)c1ccc2ccccc2c1 ; c1ccc(-c2nc(-c3cccc4c3oc3ccccc34)nc(-c3cc(-c4ccc5ccccc5c4)cc4oc5ccccc5c34)n2)cc1 ; "THF"]

Compound sub1-B-6 (15 g, 28.6 mmol) and Compound sub10 (4.9 g, 28.6 mmol) were added to 300 ml of THF under a nitrogen atmosphere, and the mixture was stirred and refluxed. Then, potassium carbonate (7.9 g, 57.3 mmol) was dissolved in 24 ml of water, added thereto, and the mixture was sufficiently stirred and then bis(tri-tert-butylphosphine)palladium(0) (0.1 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 11.4 g of Compound 1-11. (Yield: 65%, MS: [M+H]⁺=616)

### Preparation Example 1-12: Preparation of Compound 1-12

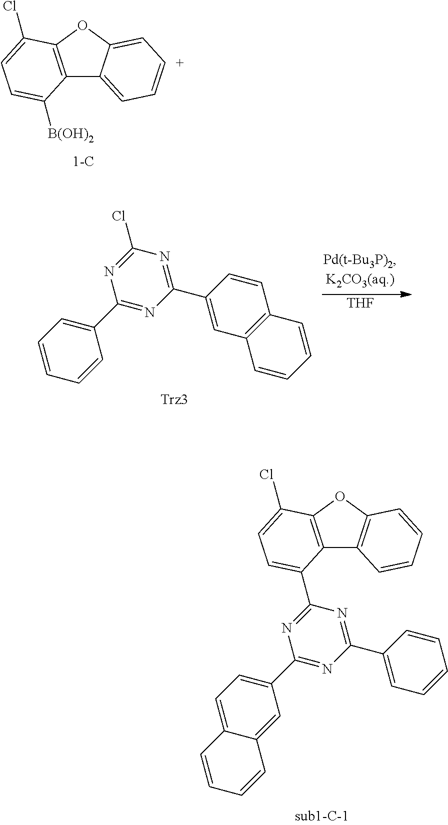 [SMILES: OB(O)c1ccc(Cl)c2oc3c(c12)=CC=[C+]C=3 ; Clc1nc(-c2ccccc2)nc(-c2ccc3ccccc3c2)n1 ; Clc1ccc(-c2nc(-c3ccccc3)nc(-c3ccc4ccccc4c3)n2)c2c1oc1ccccc12 ; "THF"]

Compound 1-C (15 g, 60.9 mmol) and Compound Trz3 (19.3 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 17.6 g of Compound sub1-C-1. (Yield: 60%, MS: [M+H]⁺=484)

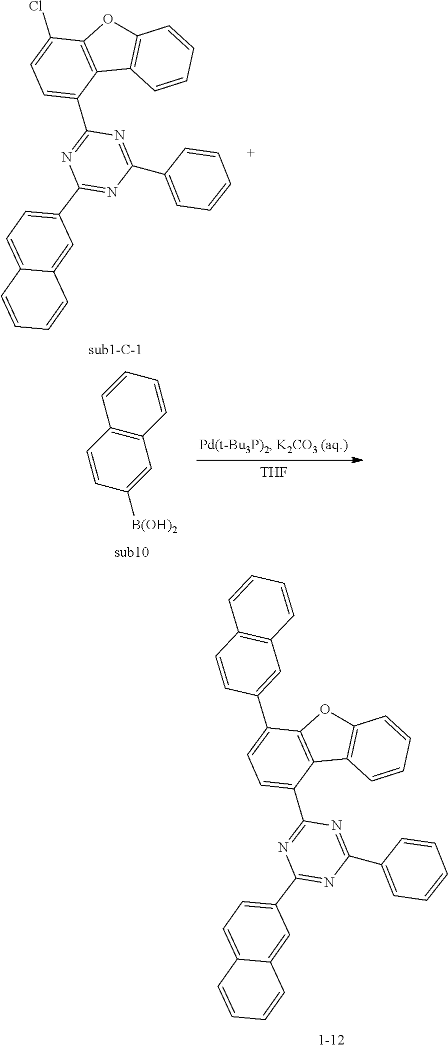 [SMILES: Clc1ccc(-c2nc(-c3ccccc3)nc(-c3ccc4ccccc4c3)n2)c2c1oc1ccccc12 ; OB(O)c1ccc2ccccc2c1 ; c1ccc(-c2nc(-c3ccc4ccccc4c3)nc(-c3ccc(-c4ccc5ccccc5c4)c4oc5ccccc5c34)n2)cc1 ; "THF"]

Compound sub1-C-1 (15 g, 31 mmol) and Compound sub10 (5.3 g, 31 mmol) were added to 300 ml of THF under a nitrogen atmosphere, and the mixture was stirred and refluxed. Then, potassium carbonate (8.6 g, 62 mmol) was dissolved in 26 ml of water, added thereto, and the mixture was sufficiently stirred and then bis(tri-tert-butylphosphine)palladium(0) (0.2 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.8 g of Compound 1-12. (Yield: 72%, MS: [M+H]⁺=576)

### Preparation Example 1-13: Preparation of Compound 1-13

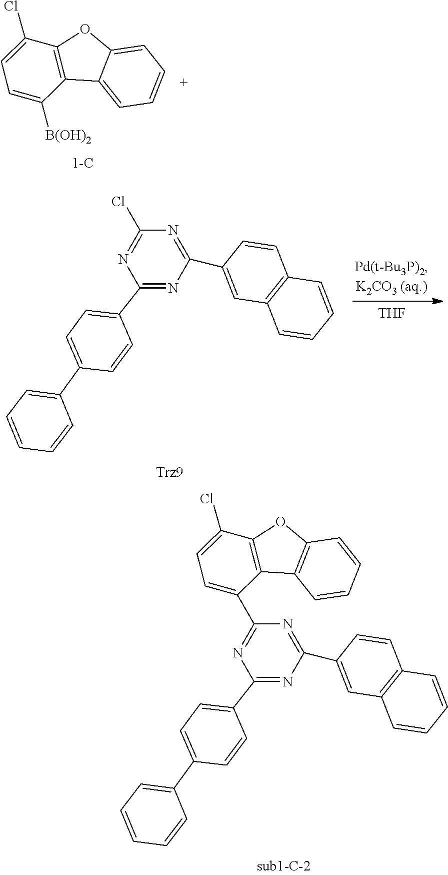 [SMILES: OB(O)c1ccc(Cl)c2oc3ccccc3c12 ; Clc1nc(-c2ccc(-c3ccccc3)cc2)nc(-c2ccc3ccccc3c2)n1 ; Clc1ccc(-c2nc(-c3ccc(-c4ccccc4)cc3)nc(-c3ccc4ccccc4c3)n2)c2c1oc1ccccc12 ; "THF"]

Compound 1-C (15 g, 60.9 mmol) and Compound Trz9 (24 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 23.5 g of Compound sub1-C-2. (Yield: 69%, MS: [M+H]⁺=560)

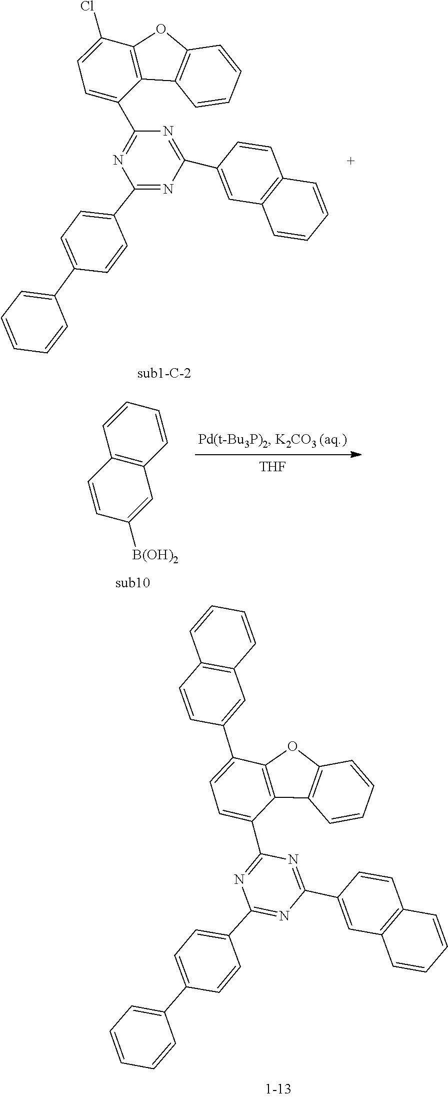 [SMILES: Clc1ccc(-c2nc(-c3ccc(-c4ccccc4)cc3)nc(-c3ccc4ccccc4c3)n2)c2c1oc1ccccc12 ; OB(O)c1ccc2ccccc2c1 ; c1ccc(-c2ccc(-c3nc(-c4ccc5ccccc5c4)nc(-c4ccc(-c5ccc6ccccc6c5)c5oc6ccccc6c45)n3)cc2)cc1 ; "THF"]

Compound sub1-C-2 (15 g, 26.8 mmol) and Compound sub10 (4.6 g, 26.8 mmol) were added to 300 ml of THF under a nitrogen atmosphere, and the mixture was stirred and refluxed. Then, potassium carbonate (7.4 g, 53.6 mmol) was dissolved in 22 ml of water, added thereto, and the mixture was sufficiently stirred and then bis(tri-tert-butylphosphine)palladium(0) (0.1 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14 g of Compound 1-13. (Yield: 80%, MS: [M+H]⁺=652)

### Preparation Example 1-14: Preparation of Compound 1-14

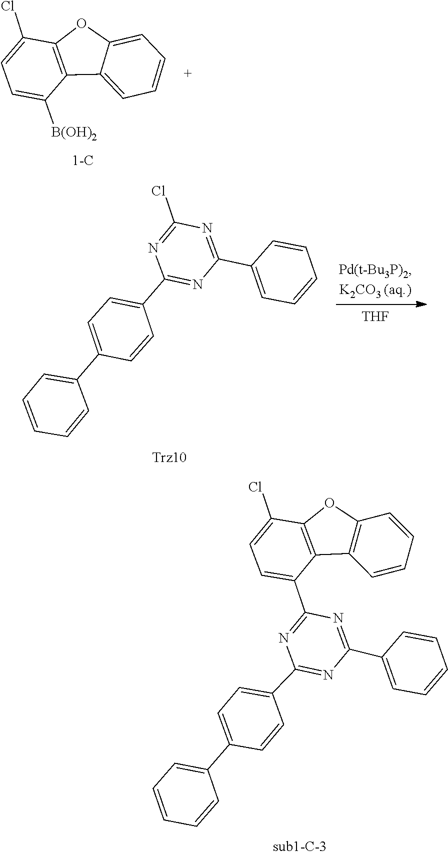 [SMILES: OB(O)c1ccc(Cl)c2oc3ccccc3c12 ; Clc1nc(-c2ccccc2)nc(-c2ccc(-c3ccccc3)cc2)n1 ; Clc1ccc(-c2nc(-c3ccccc3)nc(-c3ccc(-c4ccccc4)cc3)n2)c2c1oc1ccccc12 ; "THF"]

Compound 1-C (15 g, 60.9 mmol) and Compound Trz10 (20.9 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 20.5 g of Compound sub1-C-3. (Yield: 66%, MS: [M+H]⁺=510)

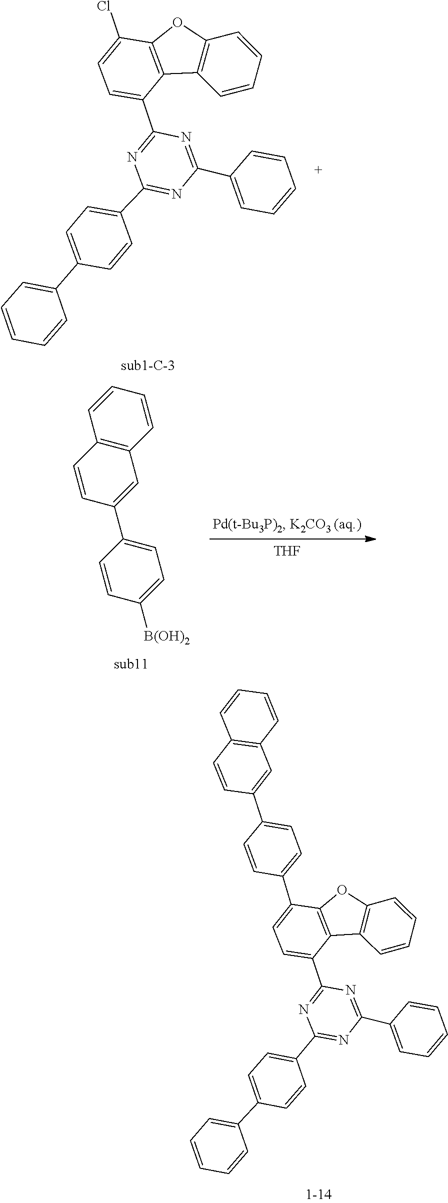 [SMILES: Clc1ccc(-c2nc(-c3ccccc3)nc(-c3ccc(-c4ccccc4)cc3)n2)c2c1oc1ccccc12 ; OB(O)c1ccc(-c2ccc3ccccc3c2)cc1 ; c1ccc(-c2ccc(-c3nc(-c4ccccc4)nc(-c4ccc(-c5ccc(-c6ccc7ccccc7c6)cc5)c5oc6ccccc6c45)n3)cc2)cc1 ; "THF"]

Compound sub1-C-3 (15 g, 29.4 mmol) and Compound sub11 (7.3 g, 29.4 mmol) were added to 300 ml of THF under a nitrogen atmosphere, and the mixture was stirred and refluxed. Then, potassium carbonate (8.1 g, 58.8 mmol) was dissolved in 24 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15.3 g of Compound 1-14. (Yield: 77%, MS: [M+H]⁺=678)

### Preparation Example 1-15: Preparation of Compound 1-15

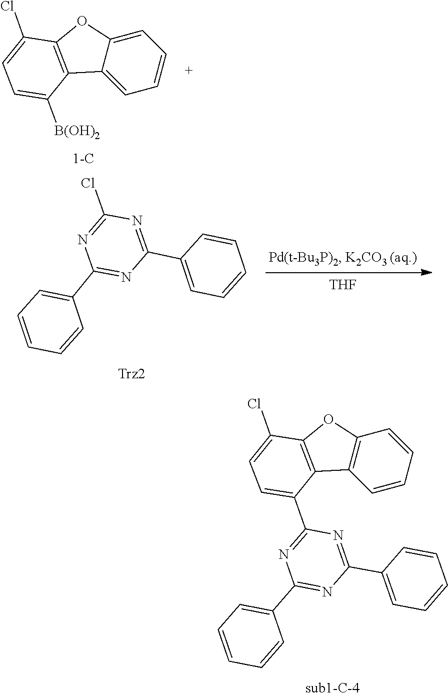 [SMILES: OB(O)c1ccc(Cl)c2oc3ccccc3c12 ; Clc1nc(-c2ccccc2)nc(-c2ccccc2)n1 ; Clc1ccc(-c2nc(-c3ccccc3)nc(-c3ccccc3)n2)c2c1oc1ccccc12 ; "THF"]

Compound 1-C (15 g, 60.9 mmol) and Compound Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 18.7 g of Compound sub1-C-4. (Yield: 71%, MS: [M+H]⁺=434)

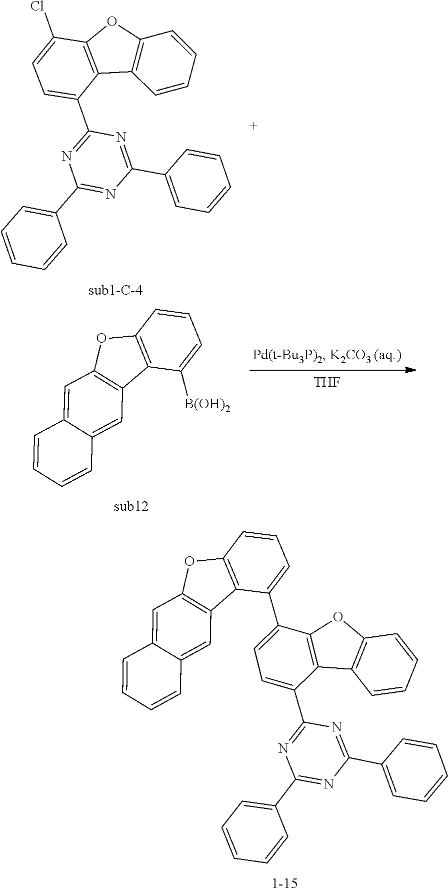 [SMILES: Clc1ccc(-c2nc(-c3ccccc3)nc(-c3ccccc3)n2)c2c1oc1ccccc12 ; OB(O)c1cccc2oc3cc4ccccc4cc3c12 ; c1ccc(-c2nc(-c3ccccc3)nc(-c3ccc(-c4cccc5oc6cc7ccccc7cc6c45)c4oc5ccccc5c34)n2)cc1 ; "THF"]

Compound sub1-C-4 (15 g, 37.1 mmol) and Compound sub12 (9.7 g, 37.1 mmol) were added to 300 ml of THF under a nitrogen atmosphere, and the mixture was stirred and refluxed. Then, potassium carbonate (10.3 g, 74.3 mmol) was dissolved in 31 ml of water, added thereto, and the mixture was sufficiently stirred and then bis(tri-tert-butylphosphine)palladium(0) (0.2 g, 0.4 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.6 g of Compound 1-15. (Yield: 64%, MS: [M+H]⁺=616)

### Preparation Example 1-16: Preparation of Compound 1-16

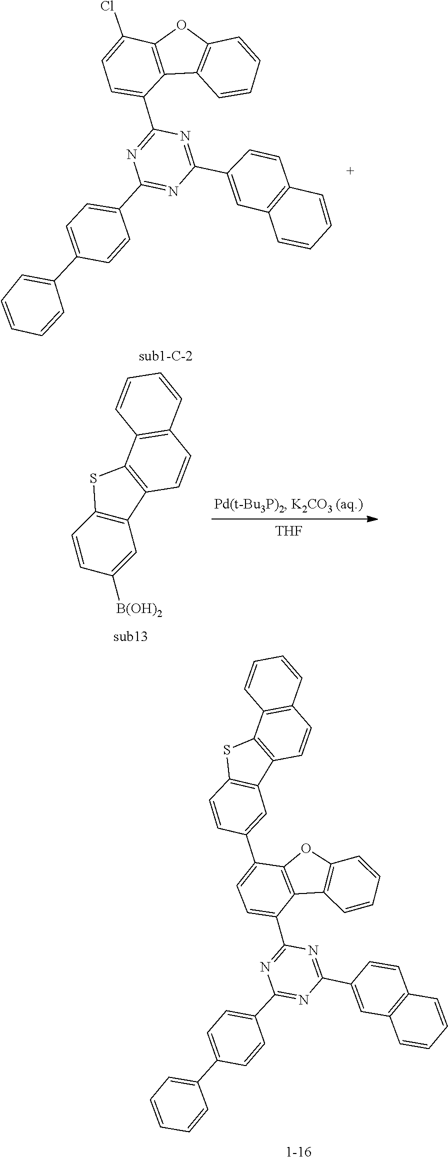 [SMILES: Clc1ccc(-c2nc(-c3ccc(-c4ccccc4)cc3)nc(-c3ccc4ccccc4c3)n2)c2c1oc1ccccc12 ; OB(O)c1ccc2sc3c4ccccc4ccc3c2c1 ; c1ccc(-c2ccc(-c3nc(-c4ccc5ccccc5c4)nc(-c4ccc(-c5ccc6sc7c8ccccc8ccc7c6c5)c5oc6ccccc6c45)n3)cc2)cc1 ; "THF"]

Compound sub1-C-2 (15 g, 26.8 mmol) and Compound sub13 (7.4 g, 26.8 mmol) were added to 300 ml of THF under a nitrogen atmosphere, and the mixture was stirred and refluxed. Then, potassium carbonate (7.4 g, 53.6 mmol) was dissolved in 22 ml of water, added thereto, and the mixture was sufficiently stirred and then bis(tri-tert-butylphosphine)palladium(0) (0.1 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 16.2 g of Compound 1-16. (Yield: 80%, MS: [M+H]⁺=758)

### Preparation Example 1-17: Preparation of Compound 1-17

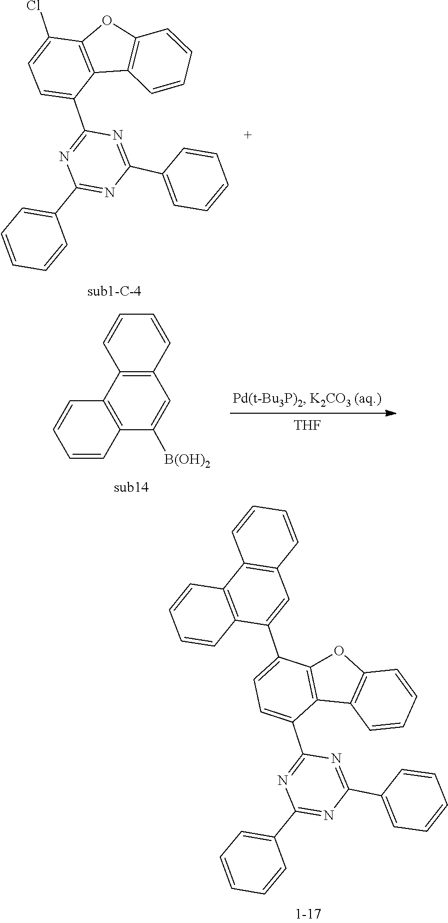 [SMILES: Clc1ccc(-c2nc(-c3ccccc3)nc(-c3ccccc3)n2)c2c1oc1ccccc12 ; OB(O)c1cc2ccccc2c2ccccc12 ; c1ccc(-c2nc(-c3ccccc3)nc(-c3ccc(-c4cc5ccccc5c5ccccc45)c4oc5ccccc5c34)n2)cc1 ; "THF"]

Compound sub1-C-4 (15 g, 34.6 mmol) and Compound sub14 (7.7 g, 34.6 mmol) were added to 300 ml of THF under a nitrogen atmosphere, and the mixture was stirred and refluxed. Then, potassium carbonate (9.6 g, 69.1 mmol) was dissolved in 29 ml of water, added thereto, and the mixture was sufficiently stirred and then bis(tri-tert-butylphosphine)palladium(0) (0.2 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.3 g of Compound 1-17. (Yield: 62%, MS: [M+H]⁺=576)

### Preparation Example 1-18: Preparation of Compound 1-18

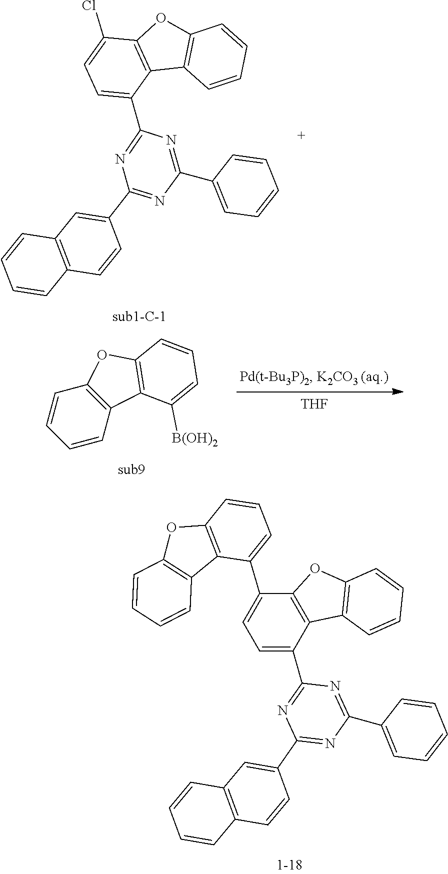 [SMILES: Clc1ccc(-c2nc(-c3ccccc3)nc(-c3ccc4ccccc4c3)n2)c2c1oc1ccccc12 ; OB(O)c1cccc2oc3ccccc3c12 ; c1ccc(-c2nc(-c3ccc4ccccc4c3)nc(-c3ccc(-c4cccc5oc6ccccc6c45)c4oc5ccccc5c34)n2)cc1 ; "THF"]

Compound sub1-C-1 (15 g, 31 mmol) and Compound sub9 (6.6 g, 31 mmol) were added to 300 ml of THF under a nitrogen atmosphere, and the mixture was stirred and refluxed. Then, potassium carbonate (8.6 g, 62 mmol) was dissolved in 26 ml of water, added thereto, and the mixture was sufficiently stirred and then bis(tri-tert-butylphosphine)palladium(0) (0.2 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12 g of Compound 1-18. (Yield: 63%, MS: [M+H]⁺=616)

### Preparation Example 1-19: Preparation of Compound 1-19

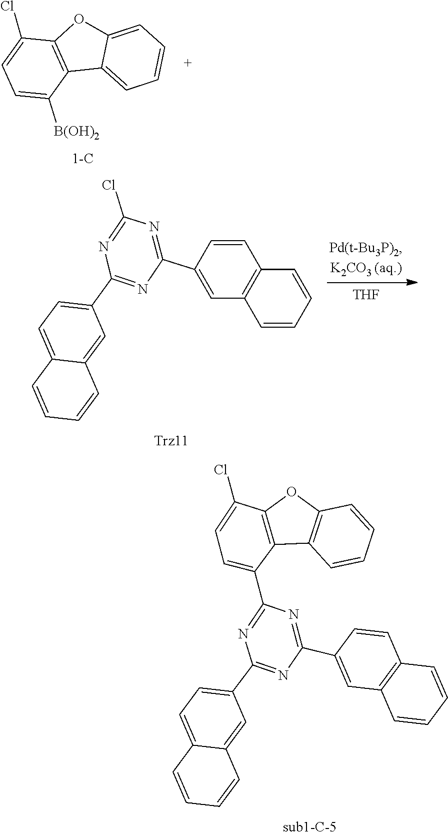 [SMILES: OB(O)c1ccc(Cl)c2oc3ccccc3c12 ; Clc1nc(-c2ccc3ccccc3c2)nc(-c2ccc3ccccc3c2)n1 ; Clc1ccc(-c2nc(-c3ccc4ccccc4c3)nc(-c3ccc4ccccc4c3)n2)c2c1oc1ccccc12 ; "THF"]

Compound 1-C (15 g, 60.9 mmol) and Compound Trz11 (22.4 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 22.4 g of Compound sub1-C-5. (Yield: 69%, MS: [M+H]⁺=534)

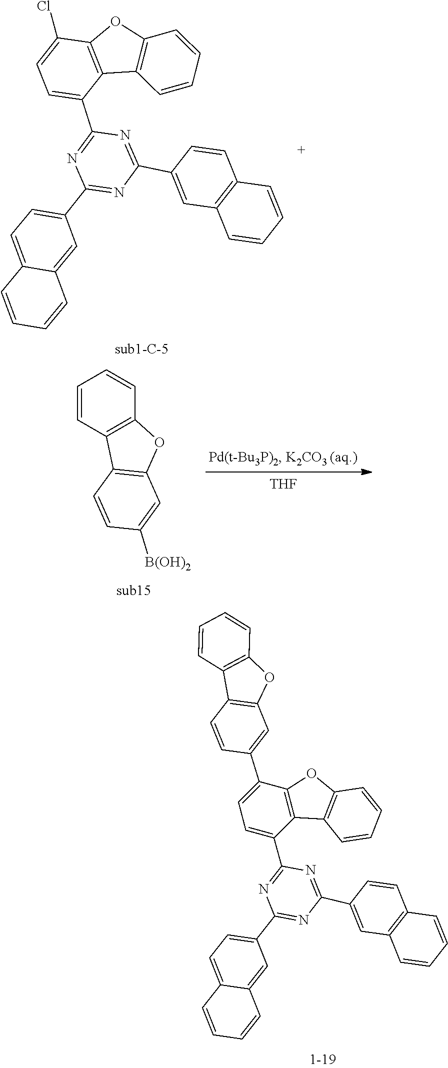 [SMILES: Clc1ccc(-c2nc(-c3ccc4ccccc4c3)nc(-c3ccc4ccccc4c3)n2)c2c1oc1ccccc12 ; OB(O)c1ccc2c(c1)oc1ccccc12 ; c1ccc2cc(-c3nc(-c4ccc5ccccc5c4)nc(-c4ccc(-c5ccc6c(c5)oc5ccccc56)c5oc6ccccc6c45)n3)ccc2c1 ; "THF"]

Compound sub1-C-5 (15 g, 28.1 mmol) and Compound sub15 (6 g, 28.1 mmol) were added to 300 ml of THF under a nitrogen atmosphere, and the mixture was stirred and refluxed. Then, potassium carbonate (7.8 g, 56.2 mmol) was dissolved in 23 ml of water, added thereto, and the mixture was sufficiently stirred and then bis(tri-tert-butylphosphine)palladium(0) (0.1 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.3 g of Compound 1-19. (Yield: 71%, MS: [M+H]⁺=666)

### Preparation Example 1-20: Preparation of Compound 1-20

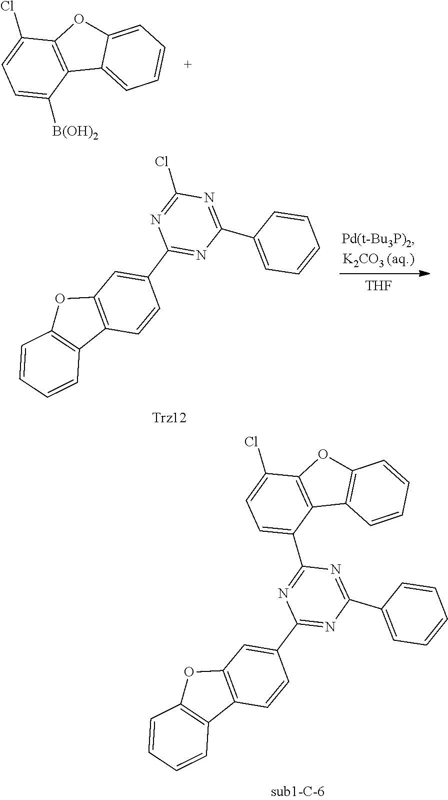 [SMILES: OB(O)c1ccc(Cl)c2oc3ccccc3c12 ; Clc1nc(-c2ccccc2)nc(-c2ccc3c(c2)oc2ccccc23)n1 ; Clc1ccc(-c2nc(-c3ccccc3)nc(-c3ccc4c(c3)oc3ccccc34)n2)c2c1oc1ccccc12 ; "THF"]

Compound 1-C (15 g, 60.9 mmol) and Compound Trz12 (21.8 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 21 g of Compound sub1-C-6. (Yield: 66%, MS: [M+H]⁺=524)

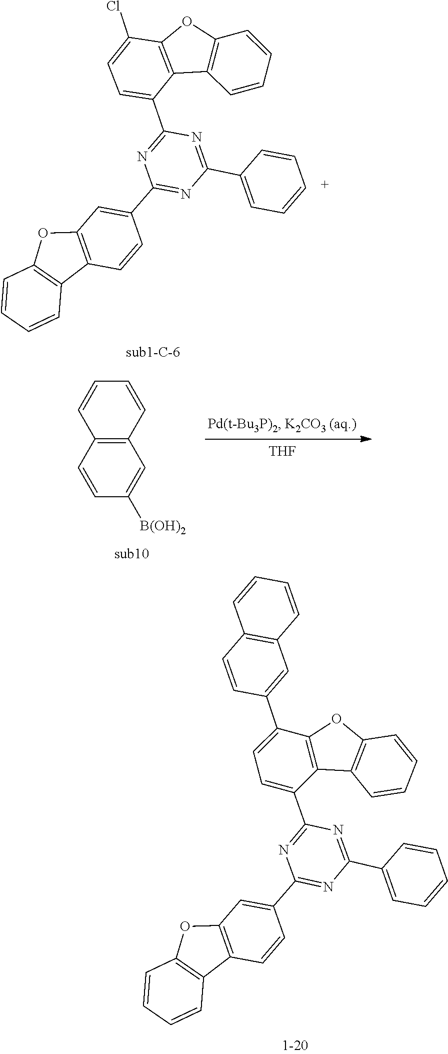 [SMILES: Clc1ccc(-c2nc(-c3ccccc3)nc(-c3ccc4c(c3)oc3ccccc34)n2)c2c1oc1ccccc12 ; OB(O)c1ccc2ccccc2c1 ; c1ccc(-c2nc(-c3ccc4c(c3)oc3ccccc34)nc(-c3ccc(-c4ccc5ccccc5c4)c4oc5ccccc5c34)n2)cc1 ; "THF"]

Compound sub1-C-6 (15 g, 28.6 mmol) and Compound sub10 (4.9 g, 28.6 mmol) were added to 300 ml of THF under a nitrogen atmosphere, and the mixture was stirred and refluxed. Then, potassium carbonate (11.9 g, 85.9 mmol) was dissolved in 36 ml of water, added thereto, and the mixture was sufficiently stirred and then bis(tri-tert-butylphosphine)palladium(0) (0.1 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.3 g of Compound 1-20. (Yield: 70%, MS: [M+H]⁺=616)

### Preparation Example 1-21: Preparation of Compound 1-21

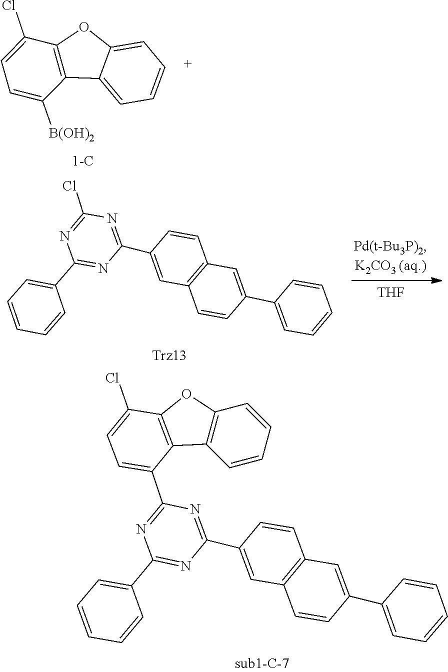 [SMILES: OB(O)c1ccc(Cl)c2oc3ccccc3c12 ; Clc1nc(-c2ccccc2)nc(-c2ccc3cc(-c4ccccc4)ccc3c2)n1 ; Clc1ccc(-c2nc(-c3ccccc3)nc(-c3ccc4cc(-c5ccccc5)ccc4c3)n2)c2c1oc1ccccc12 ; "THF"]

Compound 1-C (15 g, 60.9 mmol) and Compound Trz13 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 26.2 g of Compound sub1-C-7. (Yield: 77%, MS: [M+H]⁺=560)

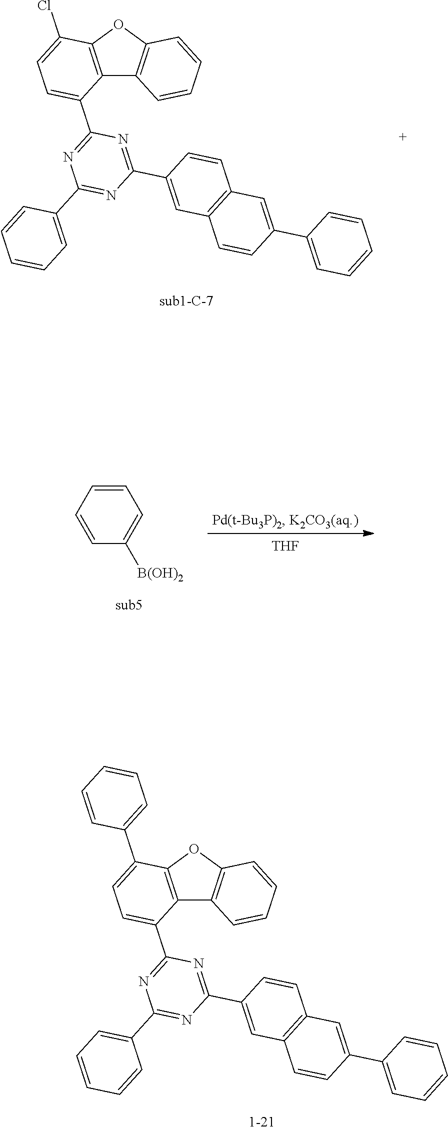 [SMILES: Clc1ccc(-c2nc(-c3ccccc3)nc(-c3ccc4cc(-c5ccccc5)ccc4c3)n2)c2c1oc1ccccc12 ; "THF" ; OB(O)c1ccccc1 ; c1ccc(-c2ccc3cc(-c4nc(-c5ccccc5)nc(-c5ccc(-c6ccccc6)c6oc7ccccc7c56)n4)ccc3c2)cc1]

Compound sub1-C-7 (15 g, 26.8 mmol) and Compound sub5 (3.3 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, added thereto, and the mixture was sufficiently stirred and then bis(tri-tert-butylphosphine)palladium(0) (0.1 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.5 g of Compound 1-21. (Yield: 65%, MS: [M+H]⁺=602)

### Preparation Example 1-22: Preparation of Compound 1-22

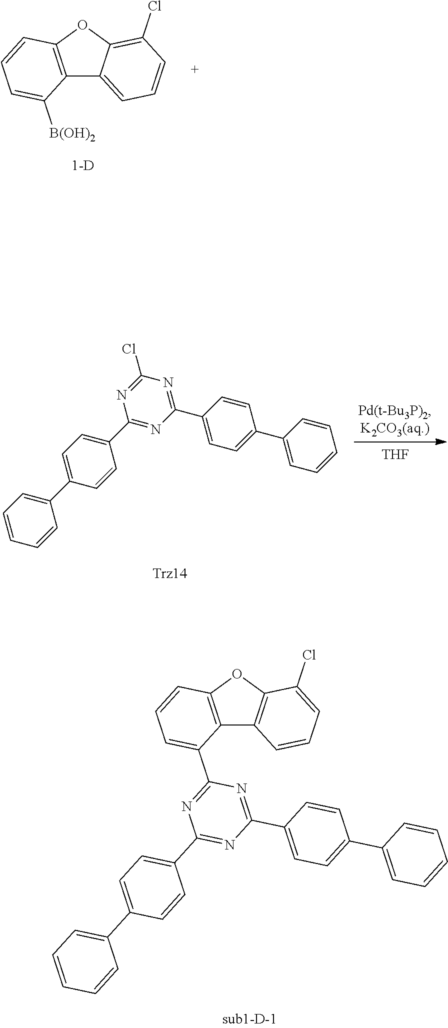 [SMILES: OB(O)c1cccc2oc3c(Cl)cccc3c12 ; Clc1nc(-c2ccc(-c3ccccc3)cc2)nc(-c2ccc(-c3ccccc3)cc2)n1 ; "THF" ; Clc1cccc2c1oc1cccc(-c3nc(-c4ccc(-c5ccccc5)cc4)nc(-c4ccc(-c5ccccc5)cc4)n3)c12]

Compound 1-D (15 g, 60.9 mmol) and Compound Trz14 (19.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 23.9 g of Compound sub1-D-1. (Yield: 67%, MS: [M+H]⁺=586)

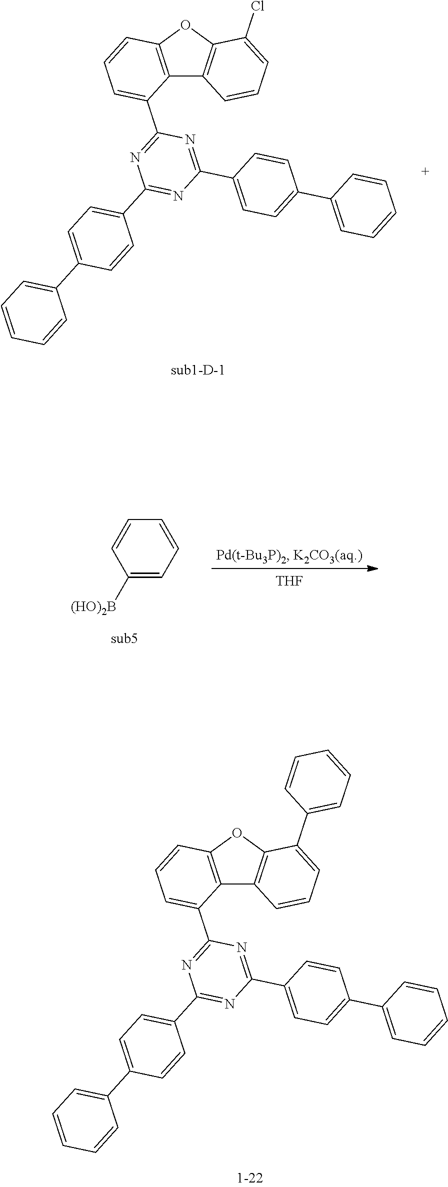 [SMILES: Clc1cccc2c1oc1cccc(-c3nc(-c4ccc(-c5ccccc5)cc4)nc(-c4ccc(-c5ccccc5)cc4)n3)c12 ; "THF" ; OB(O)c1ccccc1 ; c1ccc(-c2ccc(-c3nc(-c4ccc(-c5ccccc5)cc4)nc(-c4cccc5oc6c(-c7ccccc7)cccc6c45)n3)cc2)cc1]

Compound sub1-D-1 (15 g, 25.6 mmol) and Compound sub5 (3.1 g, 25.6 mmol) were added to 300 ml of THF under a nitrogen atmosphere, and the mixture was stirred and refluxed. Then, potassium carbonate (10.6 g, 76.8 mmol) was dissolved in 32 ml of water, added thereto, and the mixture was sufficiently stirred and then bis(tri-tert-butylphosphine)palladium(0) (0.1 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.3 g of Compound 1-22. (Yield: 64%, MS: [M+H]⁺=628)

### Preparation Example 1-23: Preparation of Compound 1-23

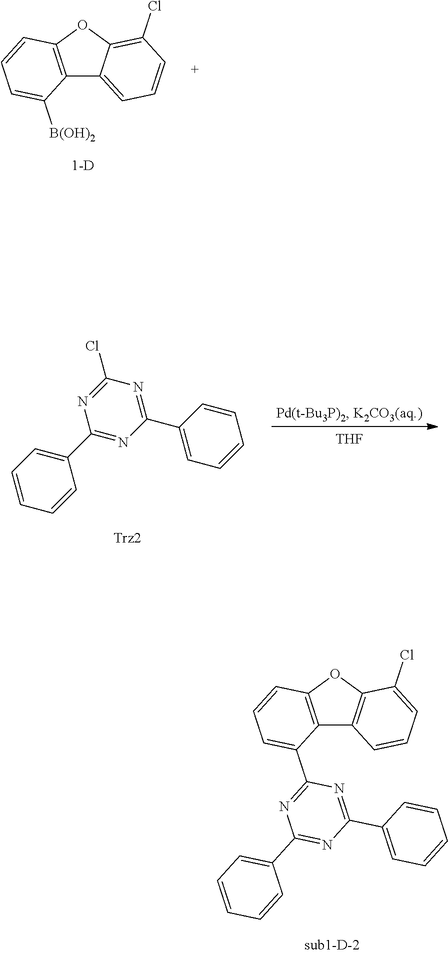 [SMILES: OB(O)c1cccc2oc3c(Cl)cccc3c12 ; Clc1nc(-c2ccccc2)nc(-c2ccccc2)n1 ; "THF" ; Clc1cccc2c1oc1cccc(-c3nc(-c4ccccc4)nc(-c4ccccc4)n3)c12]

Compound 1-D (15 g, 60.9 mmol) and Compound Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 20 g of Compound sub1-D-2. (Yield: 76%, MS: [M+H]⁺=434)

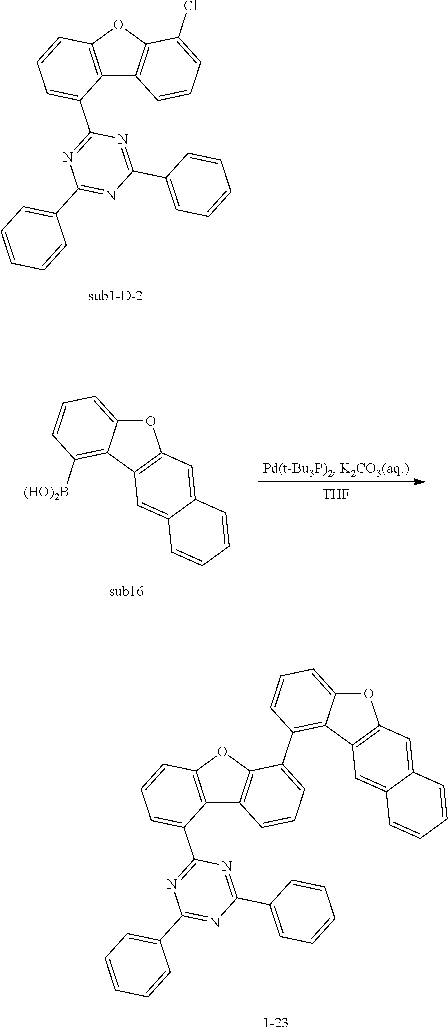 [SMILES: Clc1cccc2c1oc1cccc(-c3nc(-c4ccccc4)nc(-c4ccccc4)n3)c12 ; OB(O)c1cccc2oc3cc4ccccc4cc3c12 ; "THF" ; c1ccc(-c2nc(-c3ccccc3)nc(-c3cccc4oc5c(-c6cccc7oc8cc9ccccc9cc8c67)cccc5c34)n2)cc1]

Compound sub1-D-2 (15 g, 34.6 mmol) and Compound sub16 (9.1 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, added thereto, and the mixture was sufficiently stirred and then bis(tri-tert-butylphosphine)palladium(0) (0.2 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14 g of Compound 1-23. (Yield: 66%, MS: [M+H]⁺=616)

### Preparation Example 1-24: Preparation of Compound 1-24

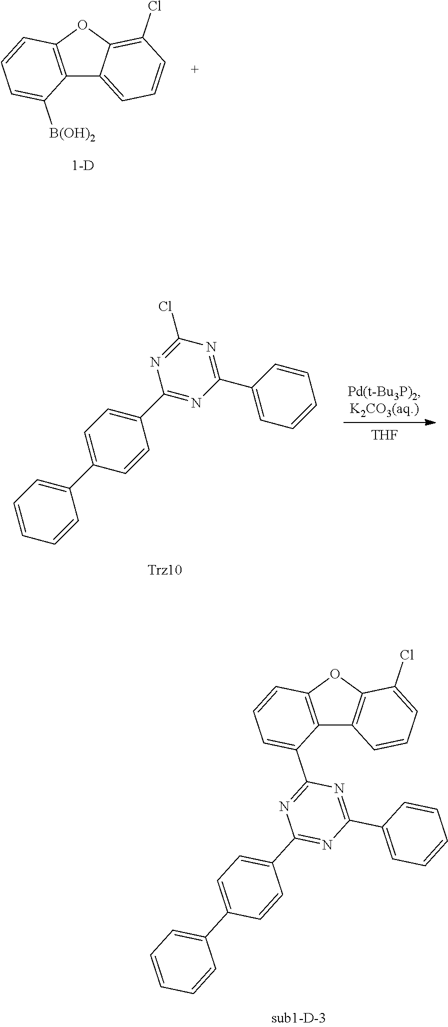 [SMILES: OB(O)c1cccc2oc3c(Cl)cccc3c12 ; Clc1nc(-c2ccccc2)nc(-c2ccc(-c3ccccc3)cc2)n1 ; "THF" ; Clc1cccc2c1oc1cccc(-c3nc(-c4ccccc4)nc(-c4ccc(-c5ccccc5)cc4)n3)c12]

Compound 1-D (15 g, 60.9 mmol) and Compound Trz10 (20.9 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 20.8 g of Compound sub1-D-3. (Yield: 67%, MS: [M+H]⁺=510)

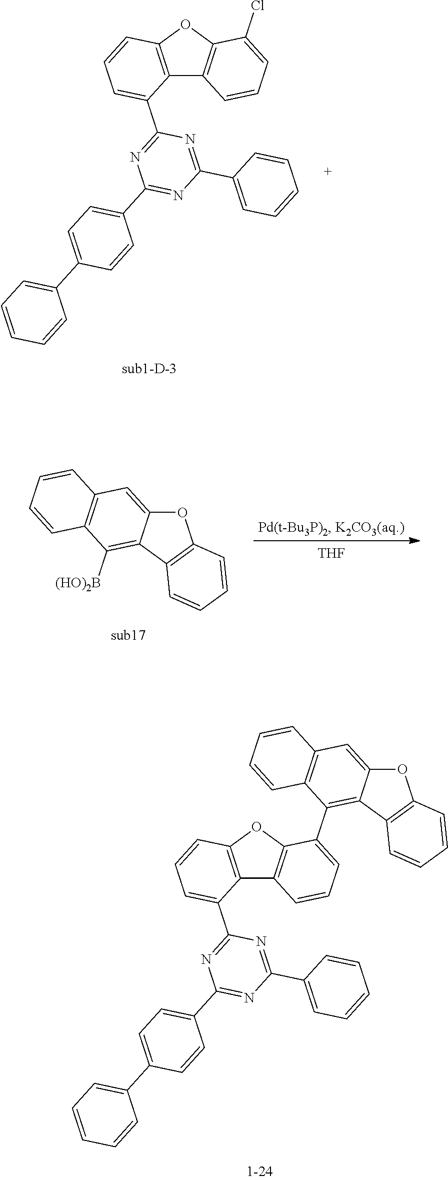 [SMILES: Clc1cccc2c1oc1cccc(-c3nc(-c4ccccc4)nc(-c4ccc(-c5ccccc5)cc4)n3)c12 ; "THF" ; OB(O)c1c2ccccc2cc2oc3ccccc3c12 ; c1ccc(-c2ccc(-c3nc(-c4ccccc4)nc(-c4cccc5oc6c(-c7c8ccccc8cc8oc9ccccc9c78)cccc6c45)n3)cc2)cc1]

Compound sub1-D-3 (15 g, 29.4 mmol) and Compound sub17 (7.7 g, 29.4 mmol) were added to 300 ml of THF under a nitrogen atmosphere, and the mixture was stirred and refluxed. Then, potassium carbonate (12.2 g, 88.2 mmol) was dissolved in 37 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.4 g of Compound 1-24. (Yield: 61%, MS: [M+H]⁺=692)

### Preparation Example 1-25: Preparation of Compound 1-25

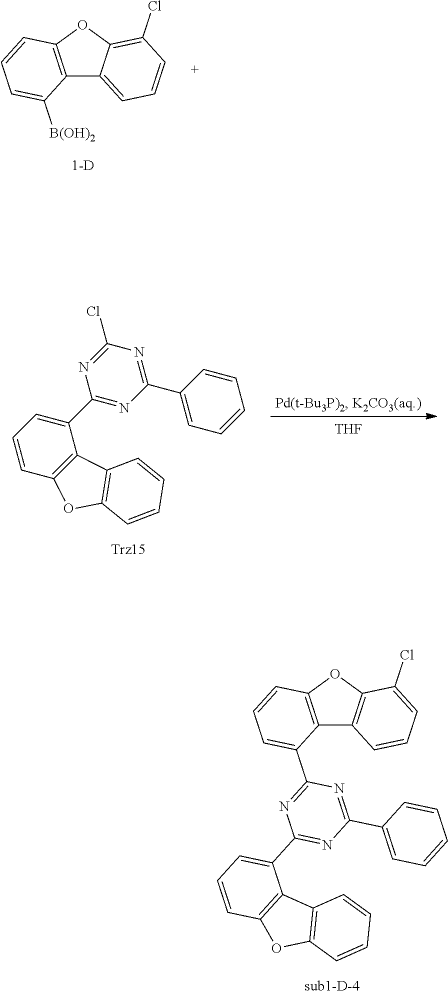 [SMILES: OB(O)c1cccc2oc3c(Cl)cccc3c12 ; Clc1nc(-c2ccccc2)nc(-c2cccc3oc4ccccc4c23)n1 ; "THF" ; Clc1cccc2c1oc1cccc(-c3nc(-c4ccccc4)nc(-c4cccc5oc6ccccc6c45)n3)c12]

Compound 1-D (15 g, 60.9 mmol) and Compound Trz15 (21.8 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 21.3 g of Compound sub1-D-4. (Yield: 67%, MS: [M+H]⁺=524)

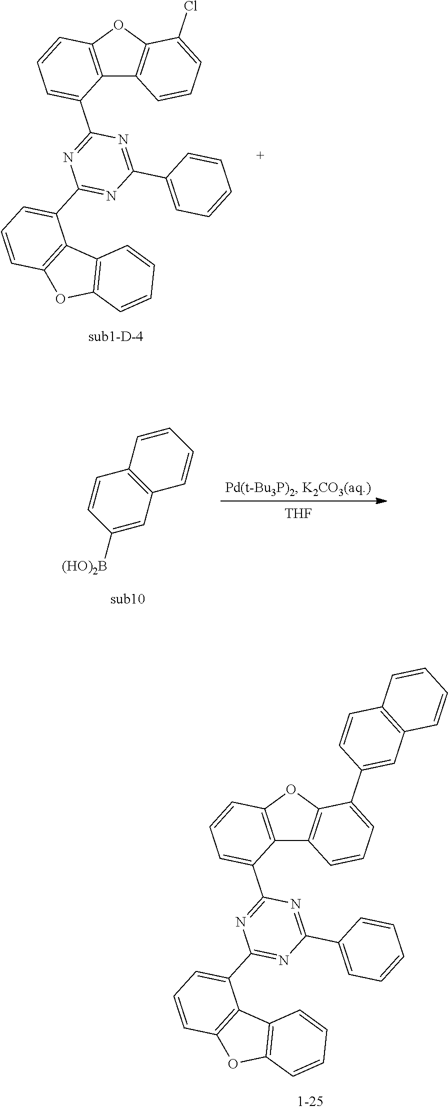 [SMILES: Clc1cccc2c1oc1cccc(-c3nc(-c4ccccc4)nc(-c4cccc5oc6ccccc6c45)n3)c12 ; "THF" ; OB(O)c1ccc2ccccc2c1 ; c1ccc(-c2nc(-c3cccc4oc5ccccc5c34)nc(-c3cccc4oc5c(-c6ccc7ccccc7c6)cccc5c34)n2)cc1]

Compound sub1-D-4 (15 g, 28.6 mmol) and Compound sub10 (4.9 g, 28.6 mmol) were added to 300 ml of THF under a nitrogen atmosphere, and the mixture was stirred and refluxed. Then, potassium carbonate (11.9 g, 85.9 mmol) was dissolved in 36 ml of water, added thereto, and the mixture was sufficiently stirred and then bis(tri-tert-butylphosphine)palladium(0) (0.1 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.7 g of Compound 1-25. (Yield: 61%, MS: [M+H]⁺=616)

### Preparation Example 1-26: Preparation of Compound 1-26

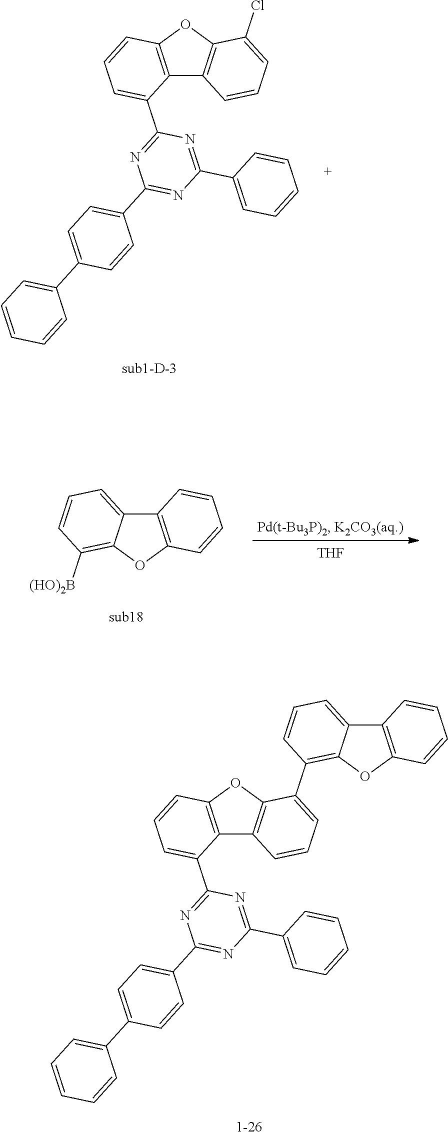 [SMILES: Clc1cccc2c1oc1cccc(-c3nc(-c4ccccc4)nc(-c4ccc(-c5ccccc5)cc4)n3)c12 ; "THF" ; OB(O)c1cccc2c1oc1ccccc12 ; c1ccc(-c2ccc(-c3nc(-c4ccccc4)nc(-c4cccc5oc6c(-c7cccc8c7oc7ccccc78)cccc6c45)n3)cc2)cc1]

Compound sub1-D-3 (15 g, 29.4 mmol) and Compound sub18 (6.2 g, 29.4 mmol) were added to 300 ml of THF under a nitrogen atmosphere, and the mixture was stirred and refluxed. Then, potassium carbonate (12.2 g, 88.2 mmol) was dissolved in 37 ml of water, added thereto, and the mixture was sufficiently stirred and then bis(tri-tert-butylphosphine)palladium(0) (0.2 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.3 g of Compound 1-26. (Yield: 76%, MS: [M+H]⁺=642)

### Preparation Example 1-27: Preparation of Compound 1-27

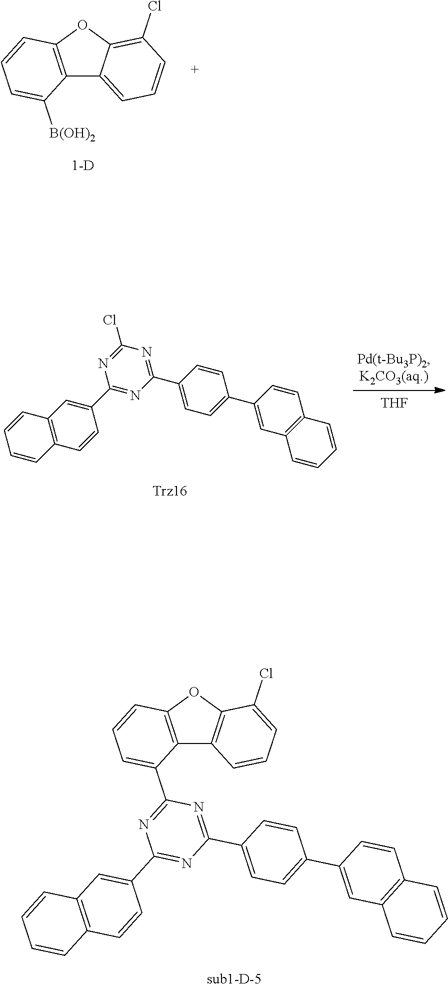 [SMILES: OB(O)c1cccc2oc3c(Cl)cccc3c12 ; "THF" ; Clc1nc(-c2ccc(-c3ccc4ccccc4c3)cc2)nc(-c2ccc3ccccc3c2)n1 ; Clc1cccc2c1oc1cccc(-c3nc(-c4ccc(-c5ccc6ccccc6c5)cc4)nc(-c4ccc5ccccc5c4)n3)c12]

Compound 1-D (15 g, 60.9 mmol) and Compound Trz16 (27 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 27.1 g of Compound sub1-D-5. (Yield: 73%, MS: [M+H]⁺=610)

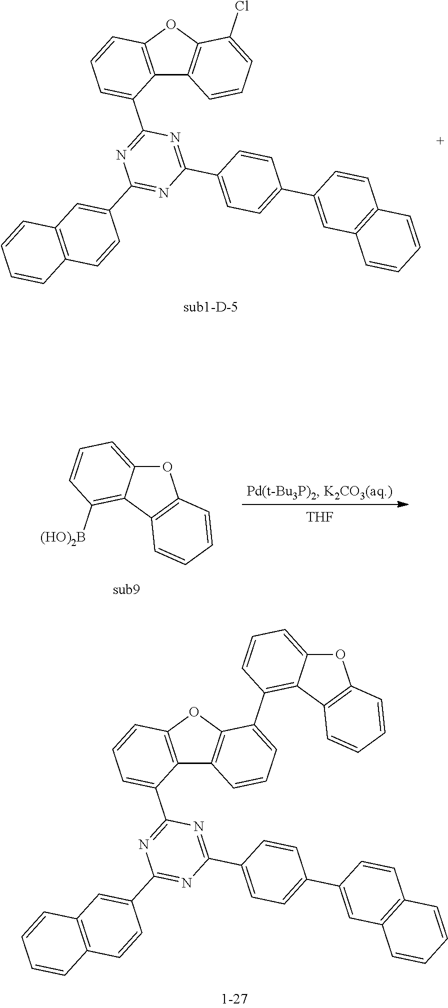 [SMILES: Clc1cccc2c1oc1cccc(-c3nc(-c4ccc(-c5ccc6ccccc6c5)cc4)nc(-c4ccc5ccccc5c4)n3)c12 ; "THF" ; OB(O)c1cccc2oc3ccccc3c12 ; c1ccc2cc(-c3ccc(-c4nc(-c5ccc6ccccc6c5)nc(-c5cccc6oc7c(-c8cccc9oc%10ccccc%10c89)cccc7c56)n4)cc3)ccc2c1]

Compound sub1-D-5 (15 g, 24.6 mmol) and Compound sub9 (5.2 g, 24.6 mmol) were added to 300 ml of THF under a nitrogen atmosphere, and the mixture was stirred and refluxed. Then, potassium carbonate (10.2 g, 73.8 mmol) was dissolved in 31 ml of water, added thereto, and the mixture was sufficiently stirred and then bis(tri-tert-butylphosphine)palladium(0) (0.1 g, 0.2 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.8 g of Compound 1-27. (Yield: 70%, MS: [M+H]⁺=742)

### Preparation Example 1-28: Preparation of Compound 1-28

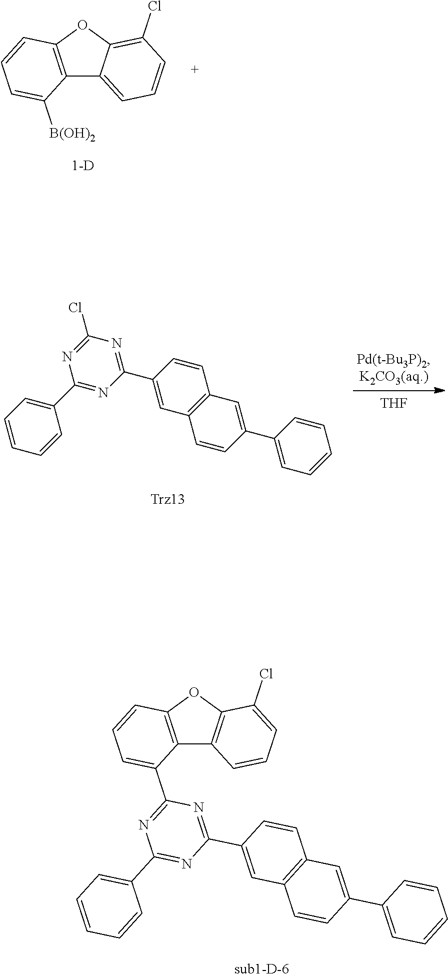 [SMILES: OB(O)c1cccc2oc3c(Cl)cccc3c12 ; Clc1nc(-c2ccccc2)nc(-c2ccc3cc(-c4ccccc4)ccc3c2)n1 ; "THF" ; Clc1cccc2c1oc1cccc(-c3nc(-c4ccccc4)nc(-c4ccc5cc(-c6ccccc6)ccc5c4)n3)c12]

Compound 1-D (15 g, 60.9 mmol) and Compound Trz13 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 20.8 g of Compound sub1-D-6. (Yield: 61%, MS: [M+H]⁺=560)

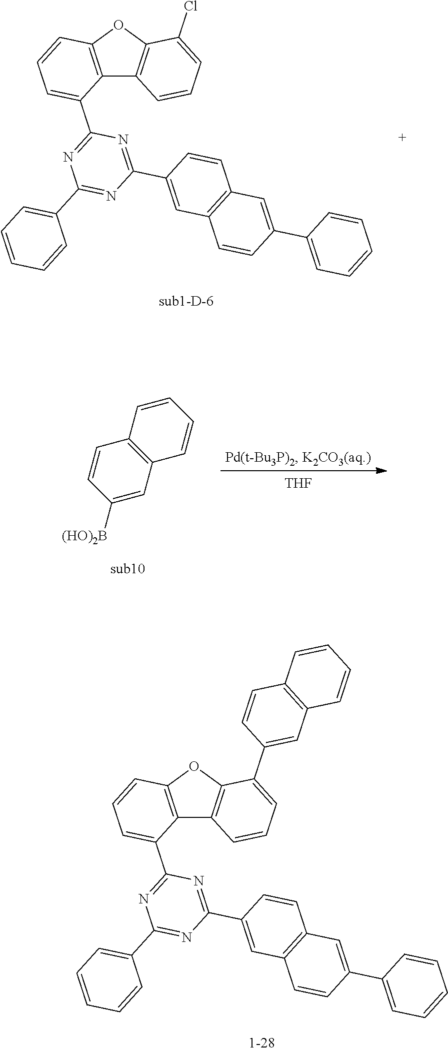 [SMILES: Clc1cccc2c1oc1cccc(-c3nc(-c4ccccc4)nc(-c4ccc5cc(-c6ccccc6)ccc5c4)n3)c12 ; "THF" ; OB(O)c1ccc2ccccc2c1 ; c1ccc(-c2ccc3cc(-c4nc(-c5ccccc5)nc(-c5cccc6oc7c(-c8ccc9ccccc9c8)cccc7c56)n4)ccc3c2)cc1]

Compound sub1-D-6 (15 g, 26.8 mmol) and Compound sub10 (4.6 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, added thereto, and the mixture was sufficiently stirred and then bis(tri-tert-butylphosphine)palladium(0) (0.1 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.2 g of Compound 1-28. (Yield: 70%, MS: [M+H]⁺=652)

### Preparation Example 1-29: Preparation of Compound 1-29

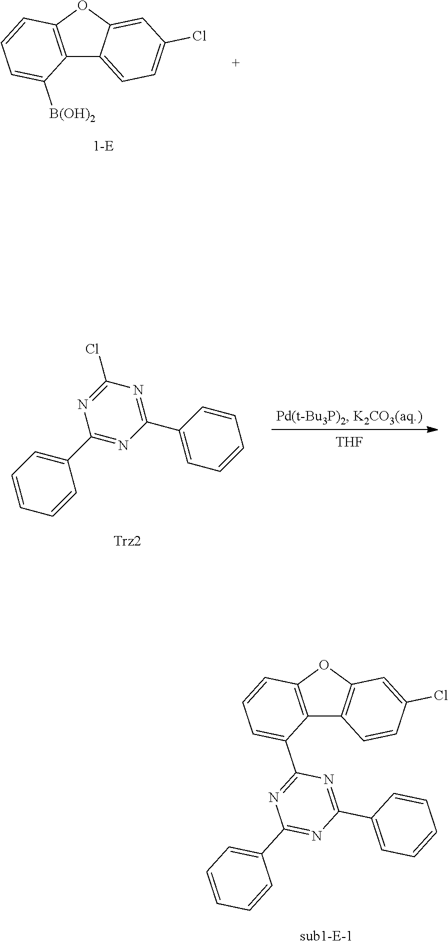 [SMILES: OB(O)c1cccc2oc3cc(Cl)ccc3c12 ; Clc1nc(-c2ccccc2)nc(-c2ccccc2)n1 ; "THF" ; Clc1ccc2c(c1)oc1cccc(-c3nc(-c4ccccc4)nc(-c4ccccc4)n3)c12]

Compound 1-E (15 g, 60.9 mmol) and Compound Trz12 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 17.1 g of Compound sub1-E-1. (Yield: 65%, MS: [M+H]⁺=434)

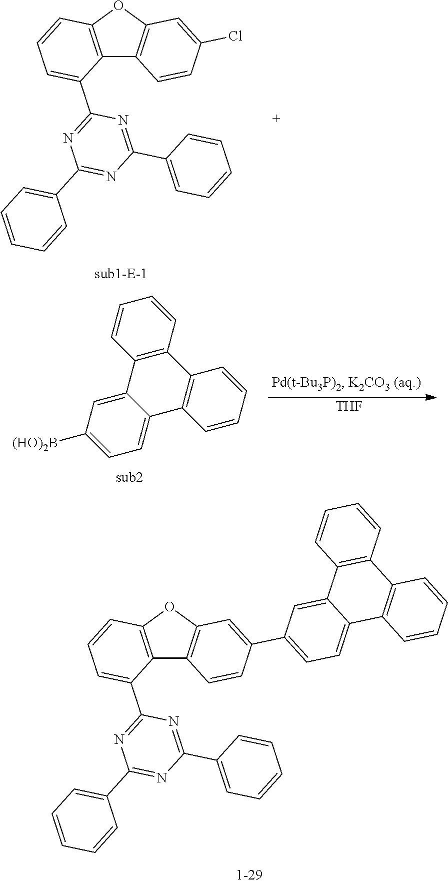 [SMILES: Clc1ccc2c(c1)oc1cccc(-c3nc(-c4ccccc4)nc(-c4ccccc4)n3)c12 ; OB(O)c1ccc2c3ccccc3c3ccccc3c2c1 ; c1ccc(-c2nc(-c3ccccc3)nc(-c3cccc4oc5cc(-c6ccc7c8ccccc8c8ccccc8c7c6)ccc5c34)n2)cc1 ; "THF"]

Compound sub1-E-1 (15 g, 34.6 mmol) and Compound sub2 (9.4 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, added thereto, and the mixture was sufficiently stirred and then bis(tri-tert-butylphosphine)palladium(0) (0.2 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.5 g of Compound 1-29. (Yield: 67%, MS: [M+H]⁺=626)

### Preparation Example 1-30: Preparation of Compound 1-30

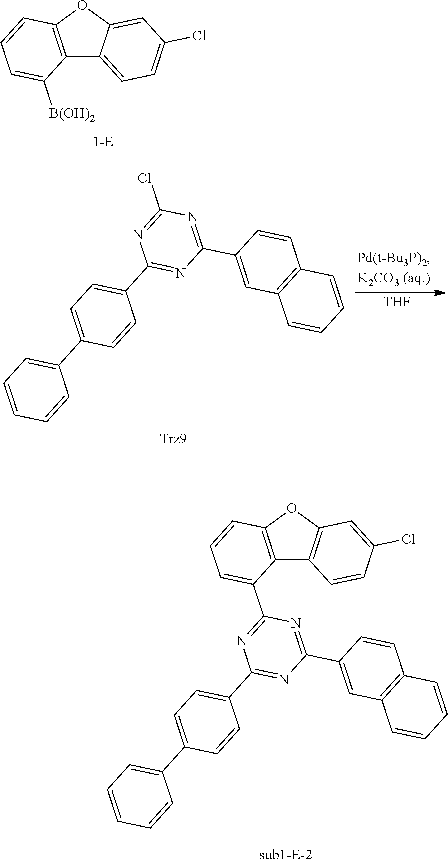 [SMILES: OB(O)c1cccc2oc3cc(Cl)ccc3c12 ; Clc1nc(-c2ccc(-c3ccccc3)cc2)nc(-c2ccc3ccccc3c2)n1 ; Clc1ccc2c(c1)oc1cccc(-c3nc(-c4ccc(-c5ccccc5)cc4)nc(-c4ccc5ccccc5c4)n3)c12 ; "THF"]

Compound 1-E (15 g, 60.9 mmol) and Compound Trz9 (24 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 26.9 g of Compound sub1-E-2. (Yield: 79%, MS: [M+H]⁺=560)

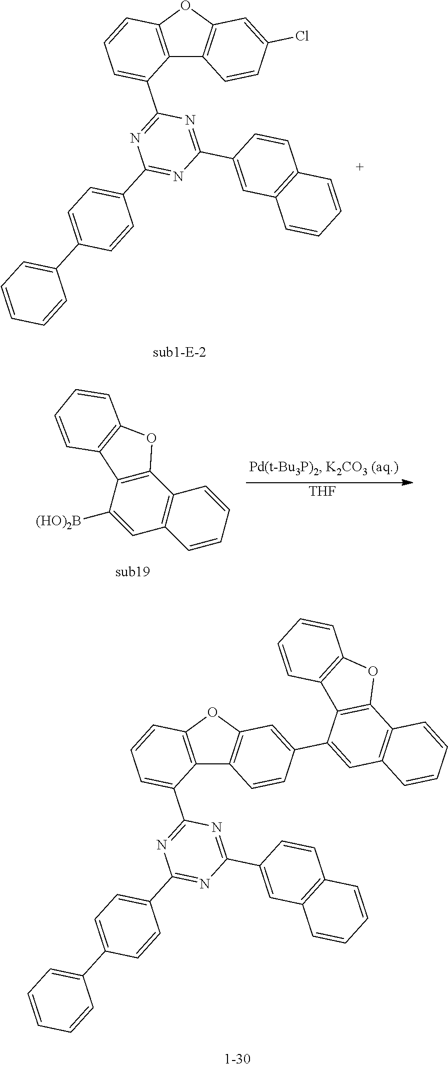 [SMILES: Clc1ccc2c(c1)oc1cccc(-c3nc(-c4ccc(-c5ccccc5)cc4)nc(-c4ccc5ccccc5c4)n3)c12 ; OB(O)c1cc2ccccc2c2oc3ccccc3c12 ; c1ccc(-c2ccc(-c3nc(-c4ccc5ccccc5c4)nc(-c4cccc5oc6cc(-c7cc8ccccc8c8oc9ccccc9c78)ccc6c45)n3)cc2)cc1 ; "THF"]

Compound sub1-E-2 (15 g, 26.8 mmol) and Compound sub19 (7 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, added thereto, and the mixture was sufficiently stirred and then bis(tri-tert-butylphosphine)palladium(0) (0.1 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15.9 g of Compound 1-30. (Yield: 80%, MS: [M+H]⁺=742)

### Preparation Example 1-31: Preparation of Compound 1-31

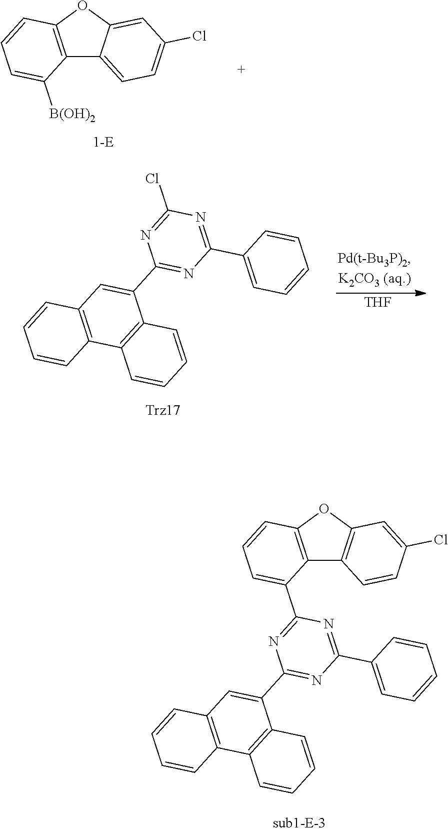 [SMILES: OB(O)c1cccc2oc3cc(Cl)ccc3c12 ; Clc1nc(-c2ccccc2)nc(-c2cc3ccccc3c3ccccc23)n1 ; Clc1ccc2c(c1)oc1cccc(-c3nc(-c4ccccc4)nc(-c4cc5ccccc5c5ccccc45)n3)c12 ; "THF"]

Compound 1-E (15 g, 60.9 mmol) and Compound Trz17 (22.4 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 25.3 g of Compound sub1-E-3. (Yield: 78%, MS: [M+H]⁺=534)

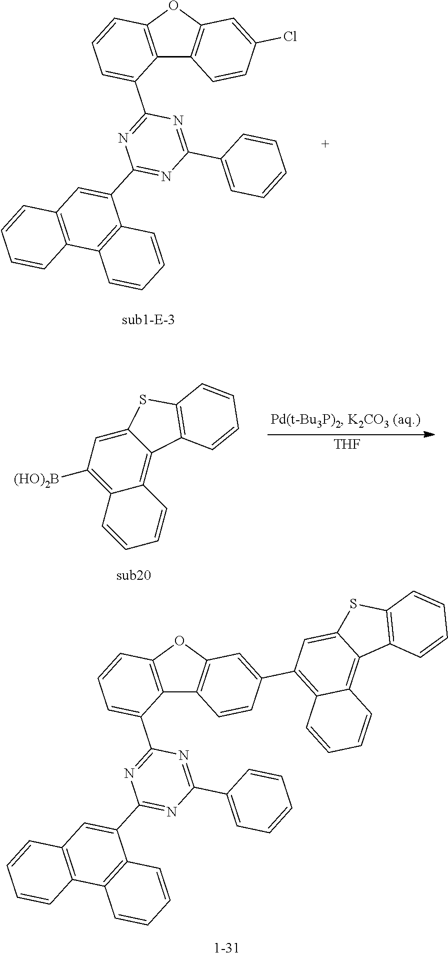 [SMILES: Clc1ccc2c(c1)oc1cccc(-c3nc(-c4ccccc4)nc(-c4cc5ccccc5c5ccccc45)n3)c12 ; OB(O)c1cc2sc3ccccc3c2c2ccccc12 ; c1ccc(-c2nc(-c3cc4ccccc4c4ccccc34)nc(-c3cccc4oc5cc(-c6cc7sc8ccccc8c7c7ccccc67)ccc5c34)n2)cc1 ; "THF"]

Compound sub1-E-3 (15 g, 28.1 mmol) and Compound sub20 (7.8 g, 28.1 mmol) were added to 300 ml of THF under a nitrogen atmosphere, and the mixture was stirred and refluxed. Then, potassium carbonate (11.6 g, 84.3 mmol) was dissolved in 35 ml of water, added thereto, and the mixture was sufficiently stirred and then bis(tri-tert-butylphosphine)palladium(0) (0.1 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.8 g of Compound 1-31. (Yield: 72%, MS: [M+H]⁺=732)

### Preparation Example 1-32: Preparation of Compound 1-32

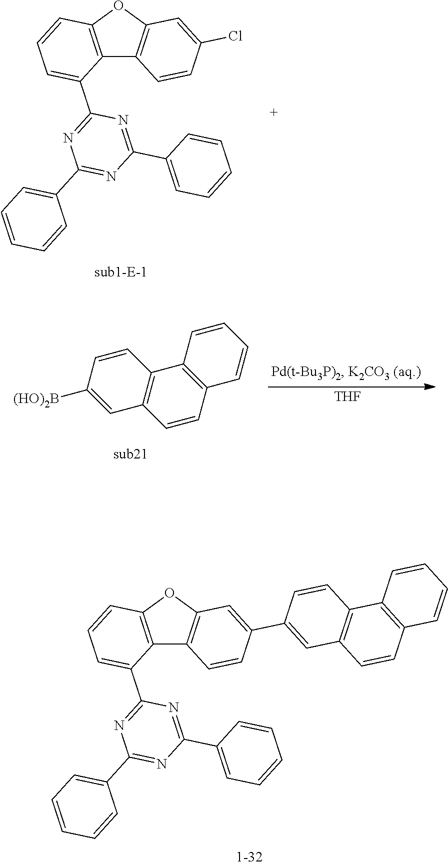 [SMILES: Clc1ccc2c(c1)oc1cccc(-c3nc(-c4ccccc4)nc(-c4ccccc4)n3)c12 ; OB(O)c1ccc2c(ccc3ccccc32)c1 ; c1ccc(-c2nc(-c3ccccc3)nc(-c3cccc4oc5cc(-c6ccc7c(ccc8ccccc87)c6)ccc5c34)n2)cc1 ; "THF"]

Compound sub1-E-1 (15 g, 34.6 mmol) and Compound sub21 (7.7 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, added thereto, and the mixture was sufficiently stirred and then bis(tri-tert-butylphosphine)palladium(0) (0.2 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.9 g of Compound 1-32. (Yield: 65%, MS: [M+H]⁺=576)

### Preparation Example 1-33: Preparation of Compound 1-33

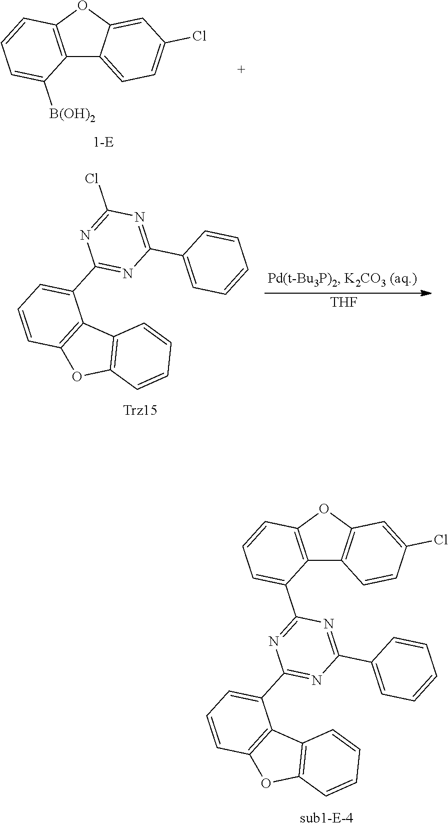 [SMILES: OB(O)c1cccc2oc3cc(Cl)ccc3c12 ; Clc1nc(-c2ccccc2)nc(-c2cccc3oc4ccccc4c23)n1 ; Clc1ccc2c(c1)oc1cccc(-c3nc(-c4ccccc4)nc(-c4cccc5oc6ccccc6c45)n3)c12 ; "THF"]

Compound 1-E (15 g, 60.9 mmol) and Compound Trz15 (21.8 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 25.5 g of Compound sub1-E-4. (Yield: 80%, MS: [M+H]⁺=524)

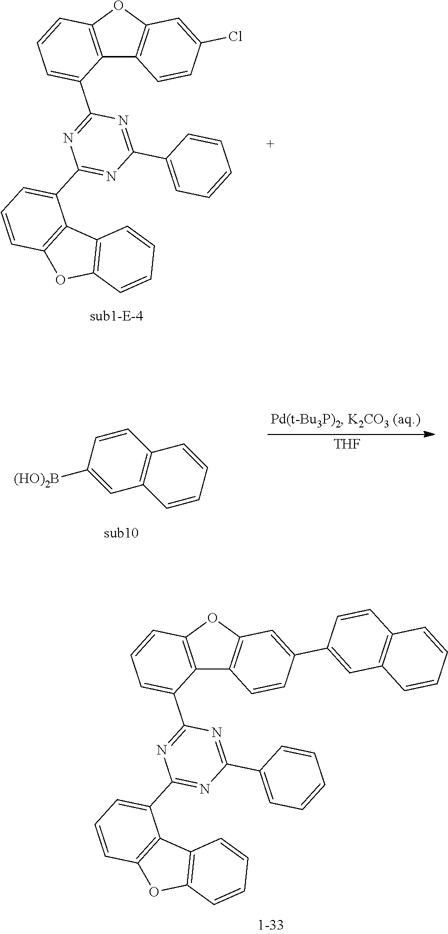 [SMILES: Clc1ccc2c(c1)oc1cccc(-c3nc(-c4ccccc4)nc(-c4cccc5oc6ccccc6c45)n3)c12 ; OB(O)c1ccc2ccccc2c1 ; c1ccc(-c2nc(-c3cccc4oc5ccccc5c34)nc(-c3cccc4oc5cc(-c6ccc7ccccc7c6)ccc5c34)n2)cc1 ; "THF"]

Compound sub1-E-4 (15 g, 28.6 mmol) and Compound sub10 (4.9 g, 28.6 mmol) were added to 300 ml of THF under a nitrogen atmosphere, and the mixture was stirred and refluxed. Then, potassium carbonate (11.9 g, 85.9 mmol) was dissolved in 36 ml of water, added thereto, and the mixture was sufficiently stirred and then bis(tri-tert-butylphosphine)palladium(0) (0.1 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.6 g of Compound 1-33. (Yield: 60%, MS: [M+H]⁺=616)

### Preparation Example 1-34: Preparation of Compound 1-34

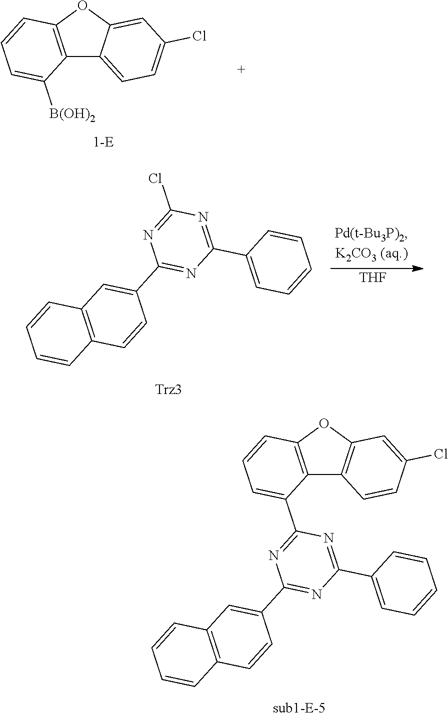 [SMILES: OB(O)c1cccc2oc3cc(Cl)ccc3c12 ; Clc1nc(-c2ccccc2)nc(-c2ccc3ccccc3c2)n1 ; Clc1ccc2c(c1)oc1cccc(-c3nc(-c4ccccc4)nc(-c4ccc5ccccc5c4)n3)c12 ; "THF"]

Compound 1-E (15 g, 60.9 mmol) and Compound Trz3 (19.3 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 17.6 g of Compound sub1-E-5. (Yield: 60%, MS: [M+H]⁺=484)

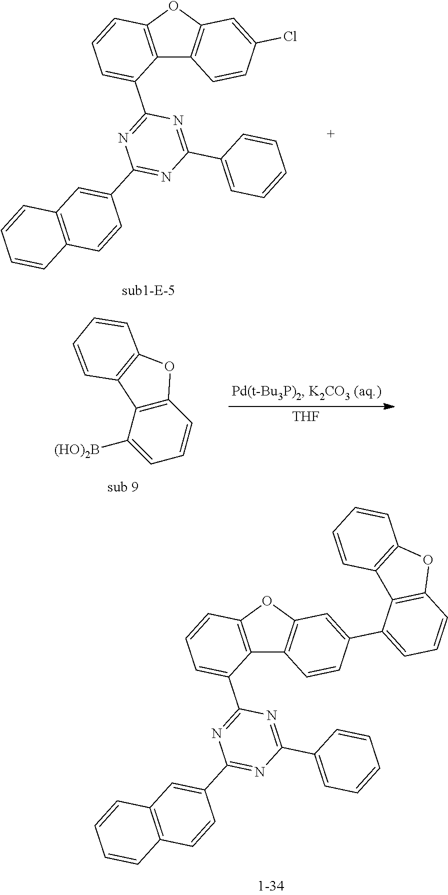 [SMILES: Clc1ccc2c(c1)oc1cccc(-c3nc(-c4ccccc4)nc(-c4ccc5ccccc5c4)n3)c12 ; OB(O)c1cccc2oc3ccccc3c12 ; c1ccc(-c2nc(-c3ccc4ccccc4c3)nc(-c3cccc4oc5cc(-c6cccc7oc8ccccc8c67)ccc5c34)n2)cc1 ; "THF"]

Compound sub1-E-5 (15 g, 31 mmol) and Compound sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, added thereto, and the mixture was sufficiently stirred and then bis(tri-tert-butylphosphine)palladium(0) (0.2 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 11.4 g of Compound 1-34. (Yield: 60%, MS: [M+H]⁺=616)

### Preparation Example 1-35: Preparation of Compound 1-35

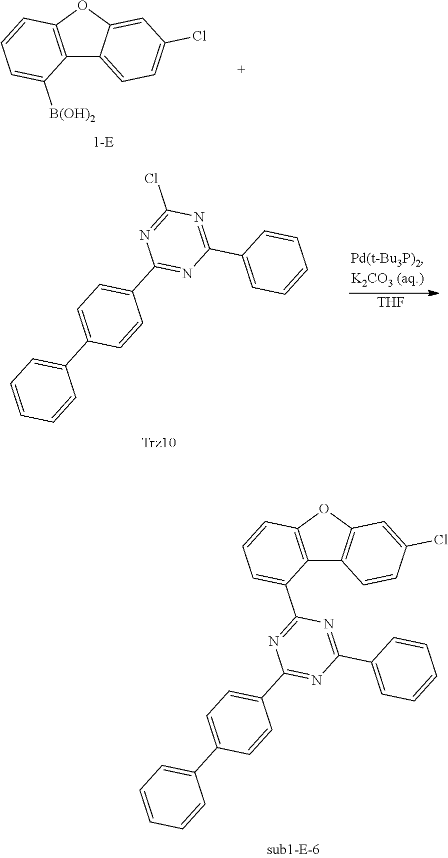 [SMILES: OB(O)c1cccc2oc3cc(Cl)ccc3c12 ; Clc1nc(-c2ccccc2)nc(-c2ccc(-c3ccccc3)cc2)n1 ; Clc1ccc2c(c1)oc1cccc(-c3nc(-c4ccccc4)nc(-c4ccc(-c5ccccc5)cc4)n3)c12 ; "THF"]

Compound 1-E (15 g, 60.9 mmol) and Compound Trz10 (20.9 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 21.7 g of Compound sub1-E-6. (Yield: 70%, MS: [M+H]⁺=510)

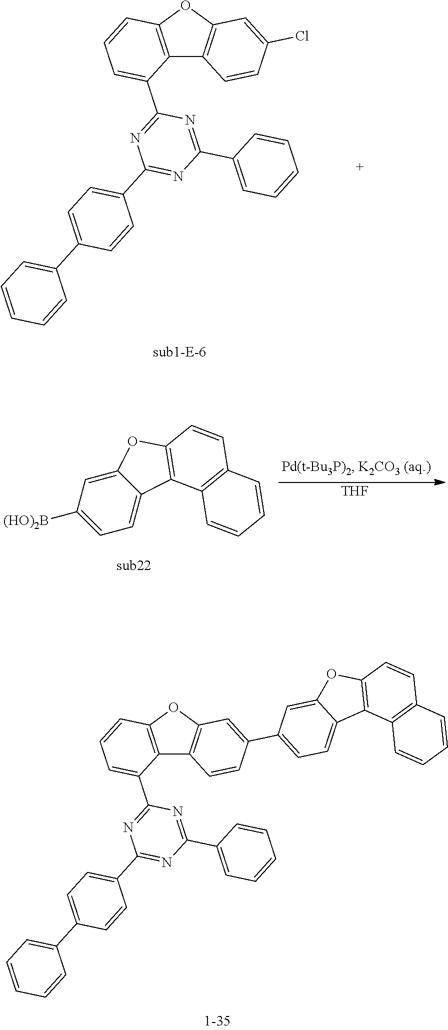 [SMILES: Clc1ccc2c(c1)oc1cccc(-c3nc(-c4ccccc4)nc(-c4ccc(-c5ccccc5)cc4)n3)c12 ; OB(O)c1ccc2c(c1)oc1ccc3ccccc3c12 ; c1ccc(-c2ccc(-c3nc(-c4ccccc4)nc(-c4cccc5oc6cc(-c7ccc8c(c7)oc7ccc9ccccc9c78)ccc6c45)n3)cc2)cc1 ; "THF"]

Compound sub1-E-6 (15 g, 29.4 mmol) and Compound sub22 (7.7 g, 29.4 mmol) were added to 300 ml of THF under a nitrogen atmosphere, and the mixture was stirred and refluxed. Then, potassium carbonate (12.2 g, 88.2 mmol) was dissolved in 37 ml of water, added thereto, and the mixture was sufficiently stirred and then bis(tri-tert-butylphosphine)palladium(0) (0.2 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.6 g of Compound 1-35. (Yield: 72%, MS: [M+H]⁺=692)

### Preparation Example 1-36: Preparation of Compound 1-36

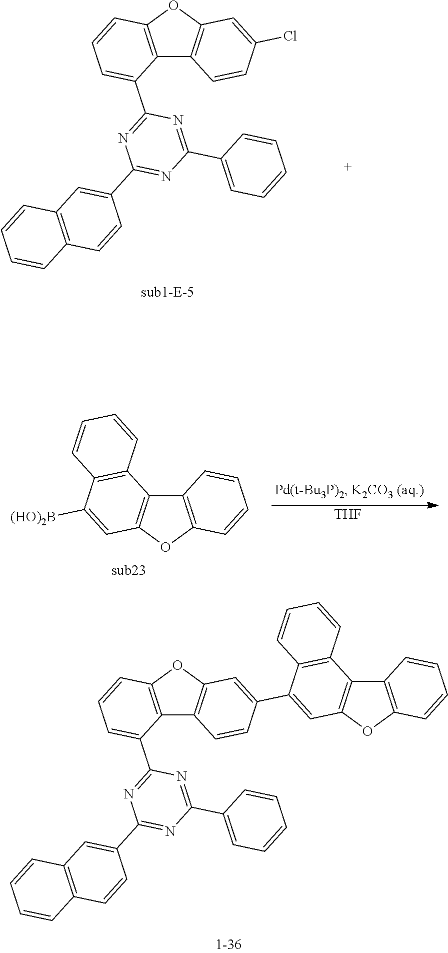 [SMILES: Clc1ccc2c(c1)oc1cccc(-c3nc(-c4ccccc4)nc(-c4ccc5ccccc5c4)n3)c12 ; OB(O)c1cc2oc3ccccc3c2c2ccccc12 ; c1ccc(-c2nc(-c3ccc4ccccc4c3)nc(-c3cccc4oc5cc(-c6cc7oc8ccccc8c7c7ccccc67)ccc5c34)n2)cc1 ; "THF"]

Compound sub1-E-5 (15 g, 31 mmol) and Compound sub23 (8.1 g, 31 mmol) were added to 300 ml of THF under a nitrogen atmosphere, and the mixture was stirred and refluxed. Then, potassium carbonate (12.9 g, 93 mmol) was dissolved in 39 ml of water, added thereto, and the mixture was sufficiently stirred and then bis(tri-tert-butylphosphine)palladium(0) (0.2 g, 0.3 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.4 g of Compound 1-36. (Yield: 60%, MS: [M+H]⁺=666)

### Preparation Example 1-37: Preparation of Compound 1-37

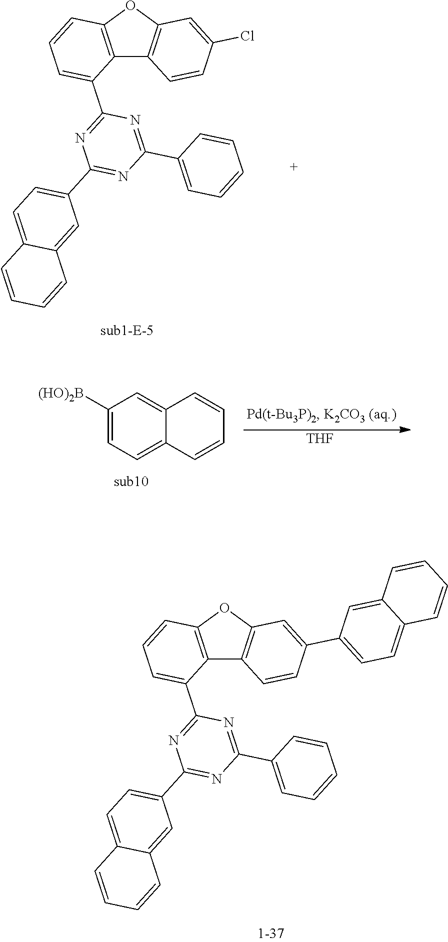 [SMILES: Clc1ccc2c(c1)oc1cccc(-c3nc(-c4ccccc4)nc(-c4ccc5ccccc5c4)n3)c12 ; OB(O)c1ccc2ccccc2c1 ; c1ccc(-c2nc(-c3ccc4ccccc4c3)nc(-c3cccc4oc5cc(-c6ccc7ccccc7c6)ccc5c34)n2)cc1 ; "THF"]

Compound sub1-E-5 (15 g, 31 mmol) and Compound sub10 (5.3 g, 31 mmol) were added to 300 ml of THF under a nitrogen atmosphere, and the mixture was stirred and refluxed. Then, potassium carbonate (12.9 g, 93 mmol) was dissolved in 39 ml of water, added thereto, and the mixture was sufficiently stirred and then bis(tri-tert-butylphosphine)palladium(0) (0.2 g, 0.3 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.1 g of Compound 1-37. (Yield: 79%, MS: [M+H]⁺=576)

### Preparation Example 1-38: Preparation of Compound 1-38

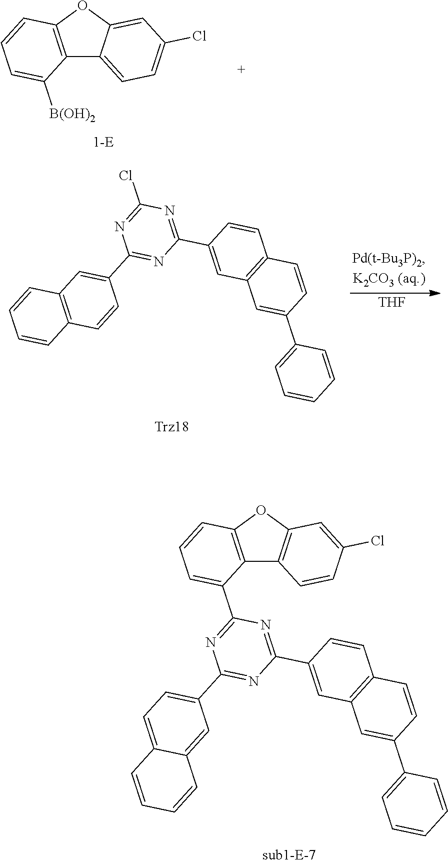 [SMILES: OB(O)c1cccc2oc3cc(Cl)ccc3c12 ; Clc1nc(-c2ccc3ccccc3c2)nc(-c2ccc3ccc(-c4ccccc4)cc3c2)n1 ; Clc1ccc2c(c1)oc1cccc(-c3nc(-c4ccc5ccccc5c4)nc(-c4ccc5ccc(-c6ccccc6)cc5c4)n3)c12 ; "THF"]

Compound 1-E (15 g, 60.9 mmol) and Compound Trz18 (27 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 24.1 g of Compound sub1-E-7. (Yield: 65%, MS: [M+H]⁺=610)

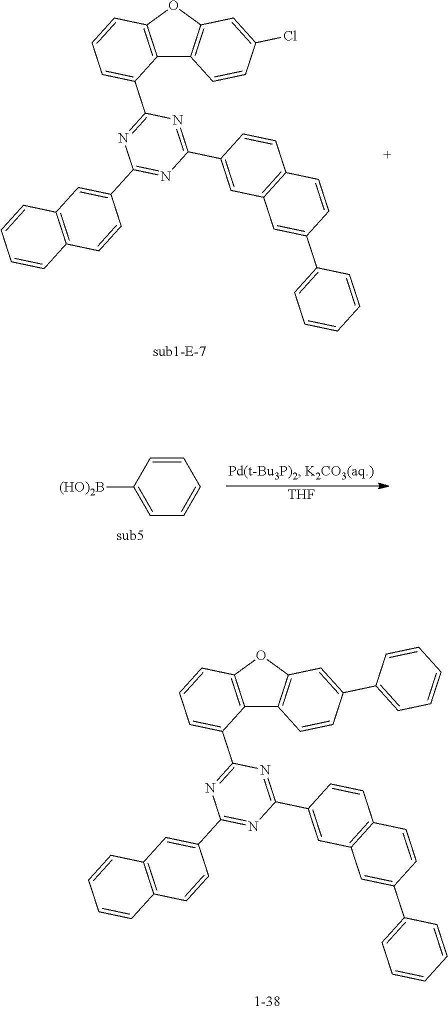 [SMILES: Clc1ccc2c(c1)oc1cccc(-c3nc(-c4ccc5ccccc5c4)nc(-c4ccc5ccc(-c6ccccc6)cc5c4)n3)c12 ; OB(O)c1ccccc1 ; "THF" ; c1ccc(-c2ccc3ccc(-c4nc(-c5ccc6ccccc6c5)nc(-c5cccc6oc7cc(-c8ccccc8)ccc7c56)n4)cc3c2)cc1]

Compound sub1-E-7 (15 g, 24.6 mmol) and Compound sub5 (3 g, 24.6 mmol) were added to 300 ml of THF under a nitrogen atmosphere, and the mixture was stirred and refluxed. Then, potassium carbonate (10.2 g, 73.8 mmol) was dissolved in 31 ml of water, added thereto, and the mixture was sufficiently stirred and then bis(tri-tert-butylphosphine)palladium(0) (0.1 g, 0.2 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.1 g of Compound 1-38. (Yield: 63%, MS: [M+H]⁺=652)

### Preparation Example 1-39: Preparation of Compound 1-39

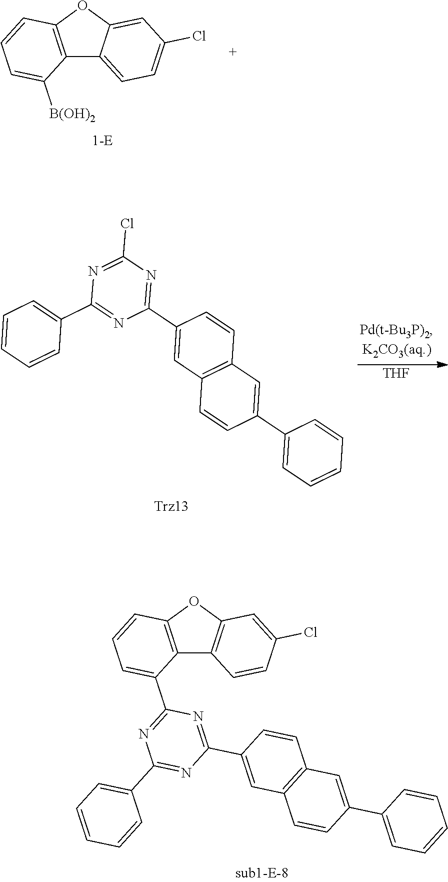 [SMILES: OB(O)c1cccc2oc3cc(Cl)ccc3c12 ; Clc1nc(-c2ccccc2)nc(-c2ccc3cc(-c4ccccc4)ccc3c2)n1 ; "THF" ; Clc1ccc2c(c1)oc1cccc(-c3nc(-c4ccccc4)nc(-c4ccc5cc(-c6ccccc6)ccc5c4)n3)c12]

Compound 1-E (15 g, 60.9 mmol) and Compound Trz13 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 26.2 g of Compound sub1-E-8. (Yield: 77%, MS: [M+H]⁺=560)

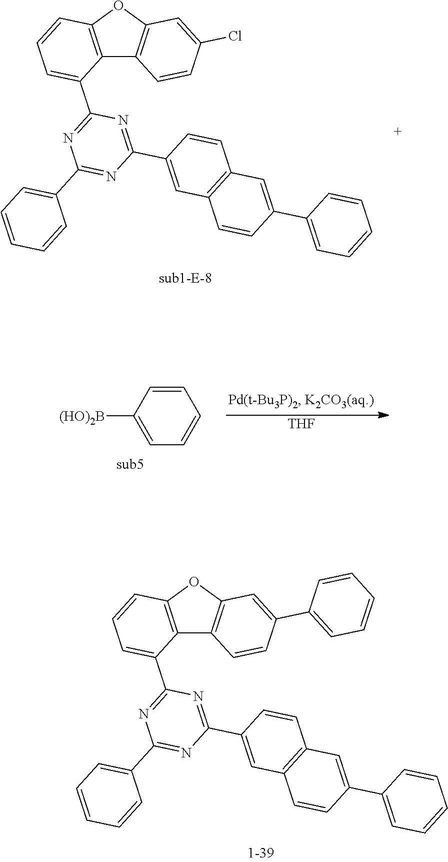 [SMILES: Clc1ccc2c(c1)oc1cccc(-c3nc(-c4ccccc4)nc(-c4ccc5cc(-c6ccccc6)ccc5c4)n3)c12 ; OB(O)c1ccccc1 ; "THF" ; c1ccc(-c2ccc3cc(-c4nc(-c5ccccc5)nc(-c5cccc6oc7cc(-c8ccccc8)ccc7c56)n4)ccc3c2)cc1]

Compound sub1-E-8 (15 g, 26.8 mmol) and Compound sub5 (3.3 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, added thereto, and the mixture was sufficiently stirred and then bis(tri-tert-butylphosphine)palladium(0) (0.1 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.9 g of Compound 1-39. (Yield: 68%, MS: [M+H]⁺=602)

### Preparation Example 1-40: Preparation of Compound 1-40

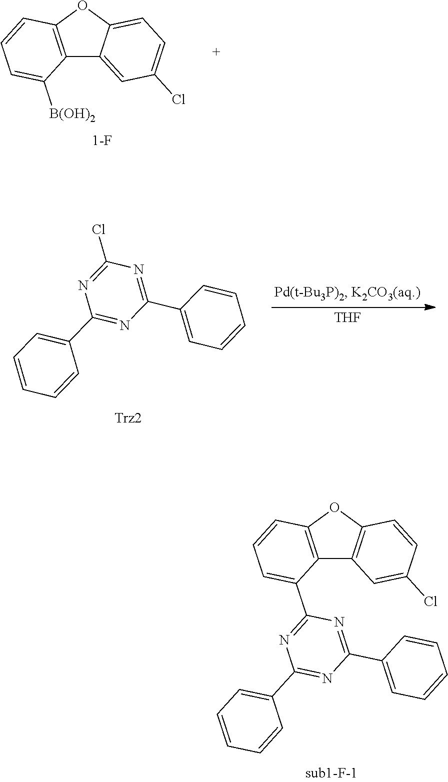 [SMILES: OB(O)c1cccc2oc3ccc(Cl)cc3c12 ; Clc1nc(-c2ccccc2)nc(-c2ccccc2)n1 ; "THF" ; Clc1ccc2oc3cccc(-c4nc(-c5ccccc5)nc(-c5ccccc5)n4)c3c2c1]

Compound 1-F (15 g, 60.9 mmol) and Compound Trz2 (16.3 g, 60.9 mmol) were added to 300 ml of THE under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 19.2 g of Compound sub1-F-1. (Yield: 73%, MS: [M+H]⁺=434)

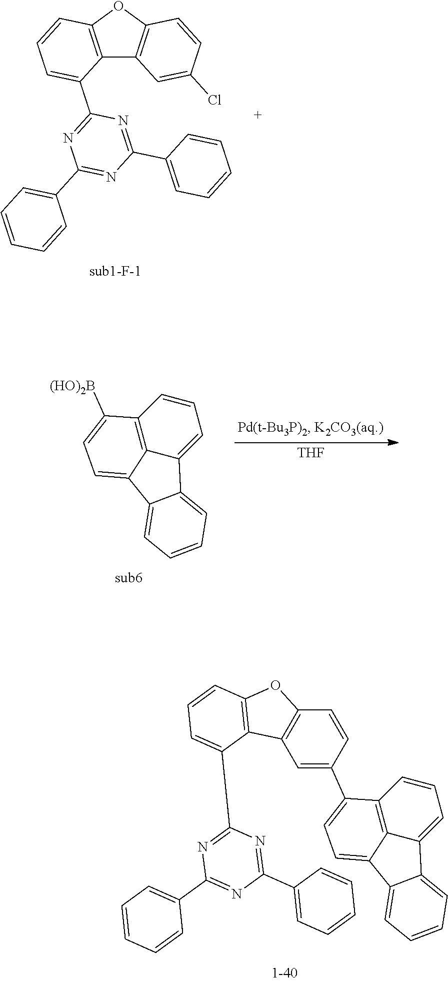 [SMILES: Clc1ccc2oc3cccc(-c4nc(-c5ccccc5)nc(-c5ccccc5)n4)c3c2c1 ; OB(O)c1ccc2c3c(cccc13)-c1ccccc1-2 ; "THF" ; c1ccc(-c2nc(-c3ccccc3)nc(-c3cccc4oc5ccc(-c6ccc7c8c(cccc68)-c6ccccc6-7)cc5c34)n2)cc1]

Compound sub1-F-1 (15 g, 34.6 mmol) and Compound sub6 (8.5 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.7 g of Compound 1-40. (Yield: 71%, MS: [M+H]⁺=600)

### Preparation Example 1-41: Preparation of Compound 1-41

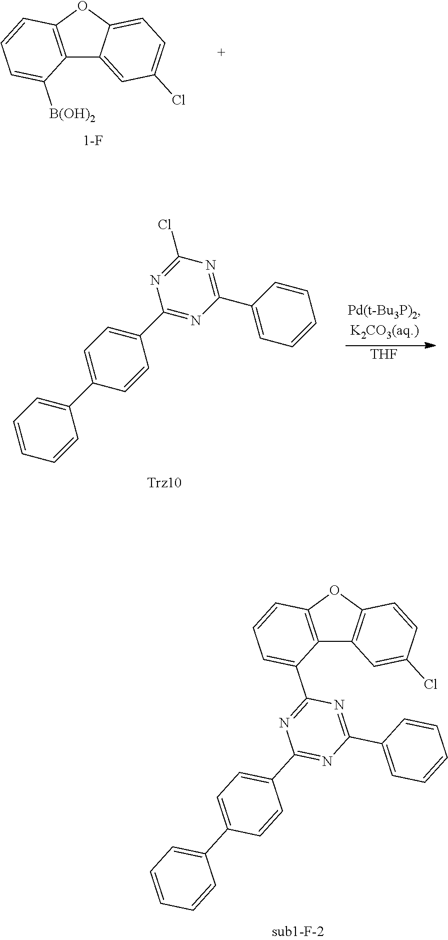 [SMILES: OB(O)c1cccc2oc3ccc(Cl)cc3c12 ; Clc1nc(-c2ccccc2)nc(-c2ccc(-c3ccccc3)cc2)n1 ; "THF" ; Clc1ccc2oc3cccc(-c4nc(-c5ccccc5)nc(-c5ccc(-c6ccccc6)cc5)n4)c3c2c1]

Compound 1-F (15 g, 60.9 mmol) and Compound Trz10 (20.9 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 21.1 g of Compound sub1-F-2. (Yield: 68%, MS: [M+H]⁺=510)

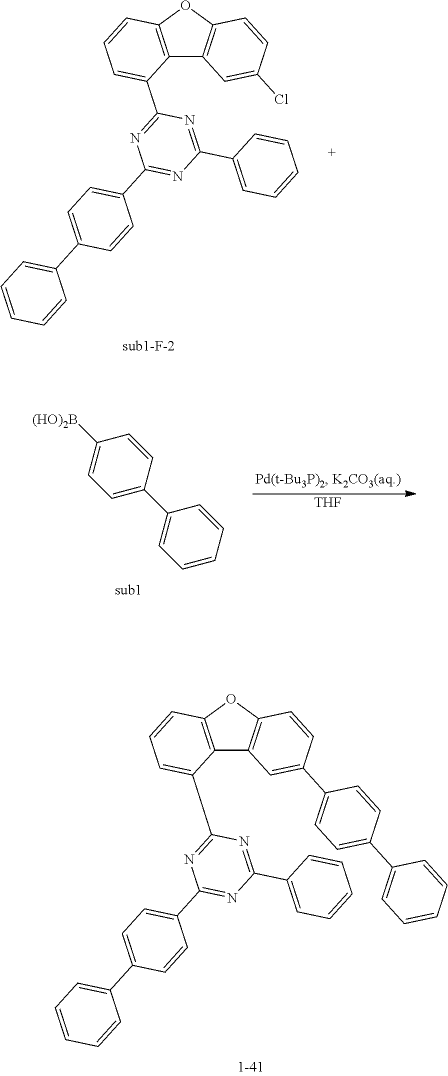 [SMILES: Clc1ccc2oc3cccc(-c4nc(-c5ccccc5)nc(-c5ccc(-c6ccccc6)cc5)n4)c3c2c1 ; OB(O)c1ccc(-c2ccccc2)cc1 ; "THF" ; c1ccc(-c2ccc(-c3ccc4oc5cccc(-c6nc(-c7ccccc7)nc(-c7ccc(-c8ccccc8)cc7)n6)c5c4c3)cc2)cc1]

Compound sub1-F-2 (15 g, 29.4 mmol) and Compound sub1 (5.8 g, 29.4 mmol) were added to 300 ml of THF under a nitrogen atmosphere, and the mixture was stirred and refluxed. Then, potassium carbonate (12.2 g, 88.2 mmol) was dissolved in 37 ml of water, added thereto, and the mixture was sufficiently stirred and then bis(tri-tert-butylphosphine)palladium(0) (0.2 g, 0.3 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.2 g of Compound 1-41. (Yield: 77%, MS: [M+H]⁺=628)

### Preparation Example 1-42: Preparation of Compound 1-42

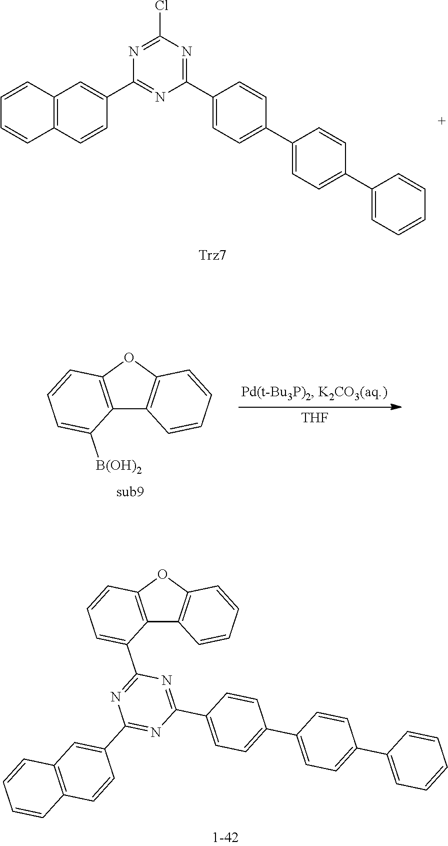 [SMILES: Clc1nc(-c2ccc(-c3ccc(-c4ccccc4)cc3)cc2)nc(-c2ccc3ccccc3c2)n1 ; OB(O)c1cccc2oc3ccccc3c12 ; c1ccc(-c2ccc(-c3ccc(-c4nc(-c5ccc6ccccc6c5)nc(-c5cccc6oc7ccccc7c56)n4)cc3)cc2)cc1 ; "THF"]

Compound Trz7 (15 g, 31.9 mmol) and Compound sub9 (6.8 g, 31.9 mmol) were added to 300 ml of THE under a nitrogen atmosphere, and the mixture was stirred and refluxed. Then, potassium carbonate (13.2 g, 95.8 mmol) was dissolved in 40 ml of water, added thereto, and the mixture was sufficiently stirred and then bis(tri-tert-butylphosphine)palladium(0) (0.2 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15.2 g of Compound 1-42. (Yield: 79%, MS: [M+H]⁺=602)

### Preparation Example 1-43: Preparation of Compound 1-43

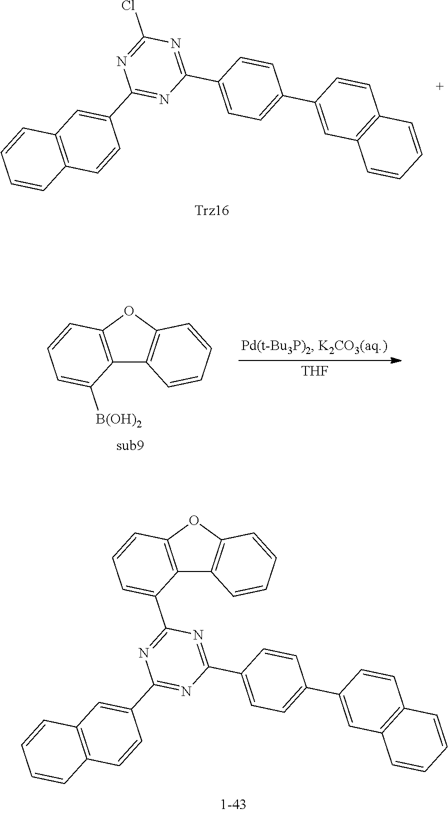

Compound Trz16 (15 g, 33.8 mmol) and Compound sub9 (7.2 g, 33.8 mmol) were added to 300 ml of THF under a nitrogen atmosphere, and the mixture was stirred and refluxed. Then, potassium carbonate (14 g, 101.4 mmol) was dissolved in 42 ml of water, added thereto, and the mixture was sufficiently stirred and then bis(tri-tert-butylphosphine)palladium(0) (0.2 g, 0.3 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15 g of Compound 1-43. (Yield: 77%, MS: [M+H]⁺=576)

### Preparation Example 1-44: Preparation of Compound 1-44

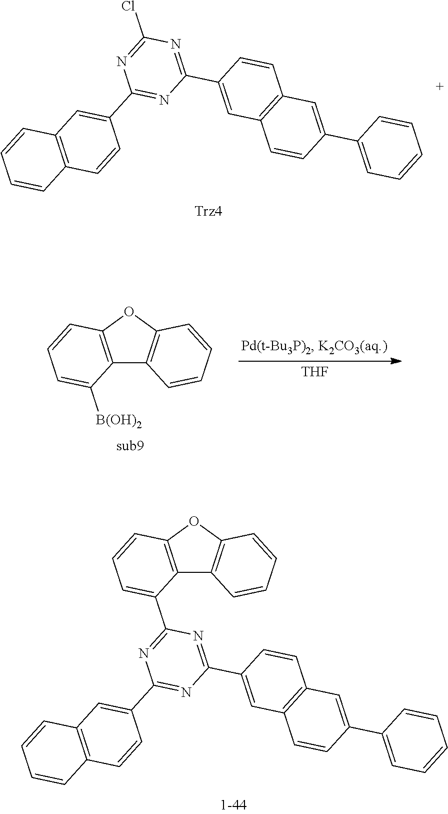

Compound Trz4 (15 g, 33.8 mmol) and Compound sub9 (7.2 g, 33.8 mmol) were added to 300 ml of THE under a nitrogen atmosphere, and the mixture was stirred and refluxed. Then, potassium carbonate (14 g, 101.4 mmol) was dissolved in 42 ml of water, added thereto, and the mixture was sufficiently stirred and then bis(tri-tert-butylphosphine)palladium(0) (0.2 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.2 g of Compound 1-44. (Yield: 73%, MS: [M+H]⁺=576)

### Preparation Example 1-45: Preparation of Compound 1-45

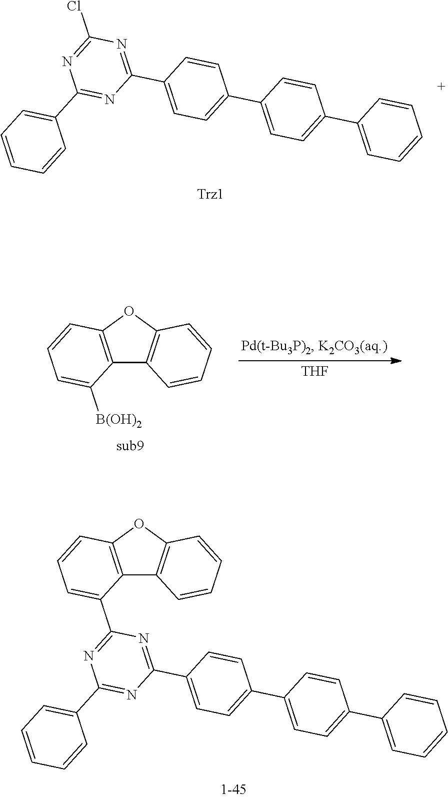

Compound Trz1 (15 g, 35.7 mmol) and Compound sub9 (7.6 g, 35.7 mmol) were added to 300 ml of THF under a nitrogen atmosphere, and the mixture was stirred and refluxed. Then, potassium carbonate (14.8 g, 107.2 mmol) was dissolved in 44 ml of water, added thereto, and the mixture was sufficiently stirred and then bis(tri-tert-butylphosphine)palladium(0) (0.2 g, 0.4 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.2 g of Compound 1-45. (Yield: 62%, MS: [M+H]⁺=552)

### Preparation Example 1-46: Preparation of Compound 1-46

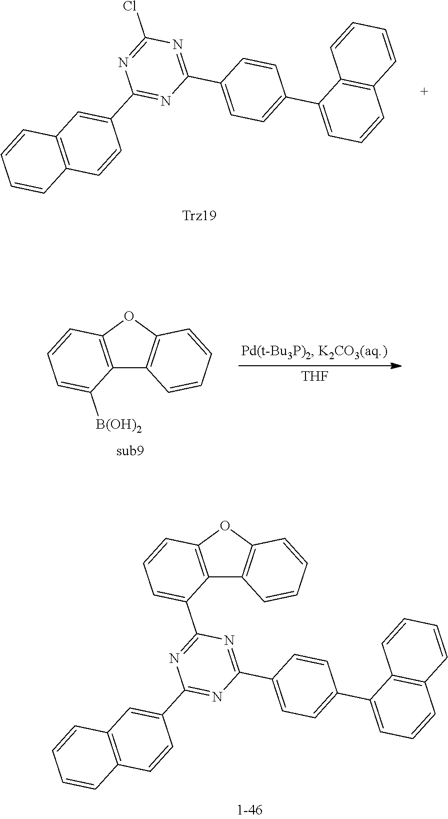 [SMILES: Clc1nc(-c2ccc(-c3cccc4ccccc34)cc2)nc(-c2ccc3ccccc3c2)n1 ; OB(O)c1cccc2oc3ccccc3c12 ; c1ccc2cc(-c3nc(-c4ccc(-c5cccc6ccccc56)cc4)nc(-c4cccc5oc6ccccc6c45)n3)ccc2c1 ; "THF"]

Compound Trz19 (15 g, 33.8 mmol) and Compound sub9 (7.2 g, 33.8 mmol) were added to 300 ml of THF under a nitrogen atmosphere, and the mixture was stirred and refluxed. Then, potassium carbonate (14 g, 101.4 mmol) was dissolved in 42 ml of water, added thereto, and the mixture was sufficiently stirred and then bis(tri-tert-butylphosphine)palladium(0) (0.2 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.6 g of Compound 1-46. (Yield: 70%, MS: [M+H]⁺=576)

### Preparation Example 1-47: Preparation of Compound 1-47

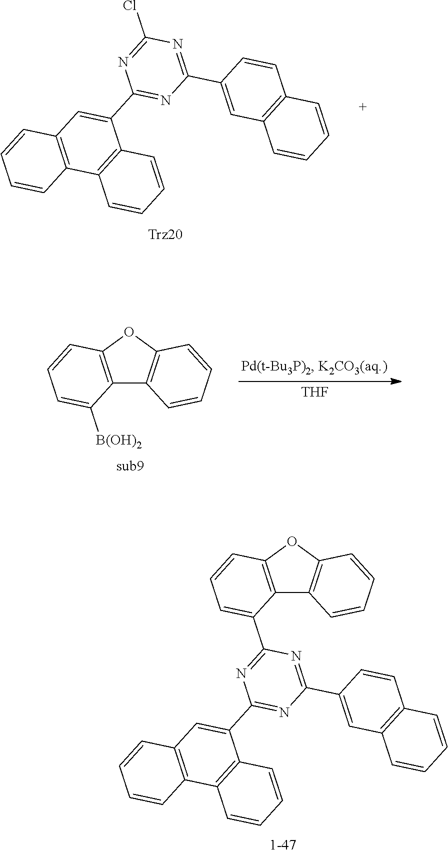 [SMILES: Clc1nc(-c2ccc3ccccc3c2)nc(-c2cc3ccccc3c3ccccc23)n1 ; OB(O)c1cccc2oc3ccccc3c12 ; c1ccc2cc(-c3nc(-c4cc5ccccc5c5ccccc45)nc(-c4cccc5oc6ccccc6c45)n3)ccc2c1 ; "THF"]

Compound Trz20 (15 g, 35.9 mmol) and Compound sub9 (7.6 g, 35.9 mmol) were added to 300 ml of THF under a nitrogen atmosphere, and the mixture was stirred and refluxed. Then, potassium carbonate (14.9 g, 107.7 mmol) was dissolved in 45 ml of water, added thereto, and the mixture was sufficiently stirred and then bis(tri-tert-butylphosphine)palladium(0) (0.2 g, 0.4 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15 g of Compound 1-47. (Yield: 76%, MS: [M+H]⁺=550)

### Preparation Example 1-48: Preparation of Compound 1-48

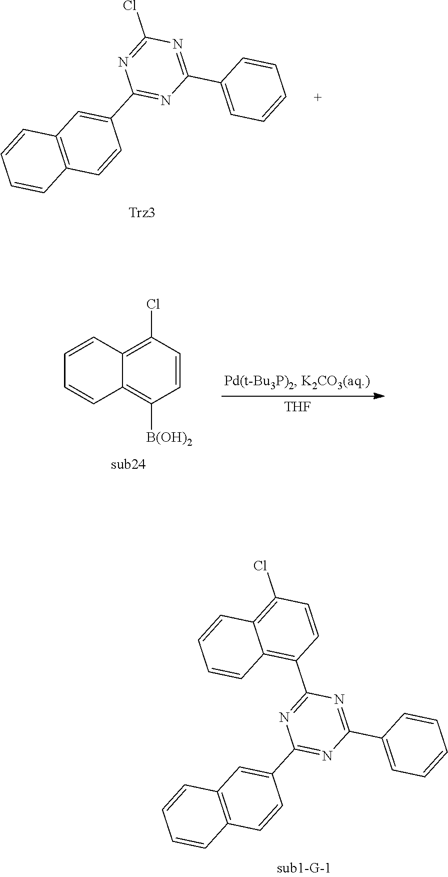 [SMILES: Clc1nc(-c2ccccc2)nc(-c2ccc3ccccc3c2)n1 ; OB(O)c1ccc(Cl)c2ccccc12 ; Clc1ccc(-c2nc(-c3ccccc3)nc(-c3ccc4ccccc4c3)n2)c2ccccc12 ; "THF"]

Compound Trz3 (15 g, 47.2 mmol) and Compound sub24 (9.7 g, 47.2 mmol) were added to 300 ml of THF under a nitrogen atmosphere, and the mixture was stirred and refluxed. Then, potassium carbonate (19.6 g, 141.6 mmol) was dissolved in 59 ml of water, added thereto, and the mixture was sufficiently stirred and then bis(tri-tert-butylphosphine)palladium(0) (0.2 g, 0.5 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13 g of Compound sub1-G-1. (Yield: 62%, MS: [M+H]⁺=444)

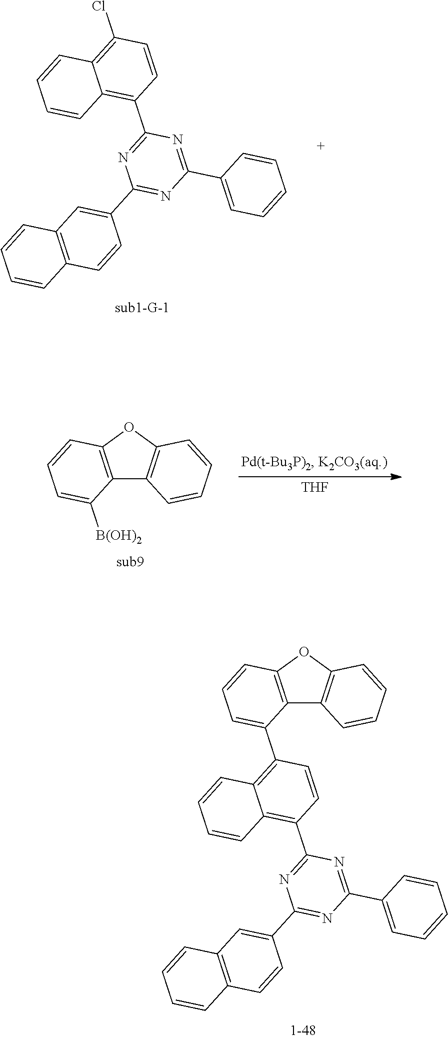 [SMILES: Clc1ccc(-c2nc(-c3ccccc3)nc(-c3ccc4ccccc4c3)n2)c2ccccc12 ; OB(O)c1cccc2oc3ccccc3c12 ; c1ccc(-c2nc(-c3ccc4ccccc4c3)nc(-c3ccc(-c4cccc5oc6ccccc6c45)c4ccccc34)n2)cc1 ; "THF"]

Compound sub1-G-1 (15 g, 33.8 mmol) and Compound sub9 (7.2 g, 33.8 mmol) were added to 300 ml of THF under a nitrogen atmosphere, and the mixture was stirred and refluxed. Then, potassium carbonate (14 g, 101.4 mmol) was dissolved in 42 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15.2 g of Compound 1-48. (Yield: 78%, MS: [M+H]⁺=576)

### Preparation Example 1-49: Preparation of Compound 1-49

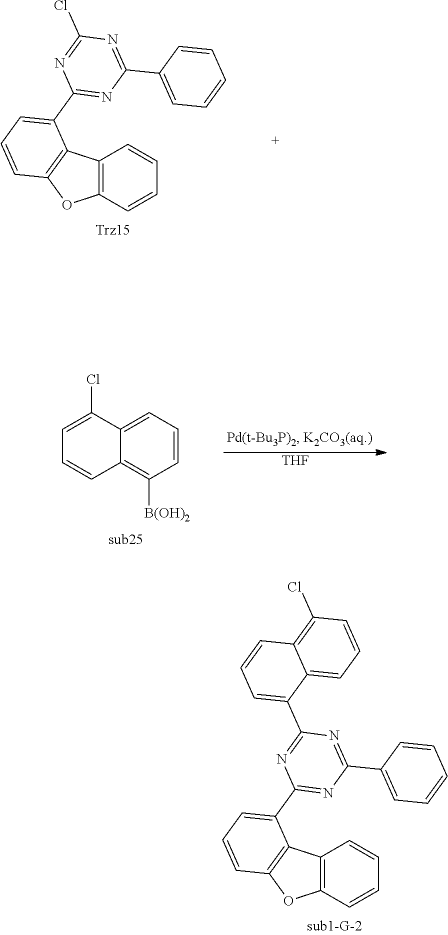 [SMILES: Clc1nc(-c2ccccc2)nc(-c2cccc3oc4ccccc4c23)n1 ; OB(O)c1cccc2c(Cl)cccc12 ; Clc1cccc2c(-c3nc(-c4ccccc4)nc(-c4cccc5oc6ccccc6c45)n3)cccc12 ; "THF"]

Compound Trz15 (15 g, 41.9 mmol) and Compound sub25 (8.7 g, 41.9 mmol) were added to 300 ml of THF under a nitrogen atmosphere, and the mixture was stirred and refluxed. Then, potassium carbonate (17.4 g, 125.8 mmol) was dissolved in 52 ml of water, added thereto, and the mixture was sufficiently stirred and then bis(tri-tert-butylphosphine)palladium(0) (0.2 g, 0.4 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.6 g of Compound sub1-G-2. (Yield: 62%, MS: [M+H]⁺=484)

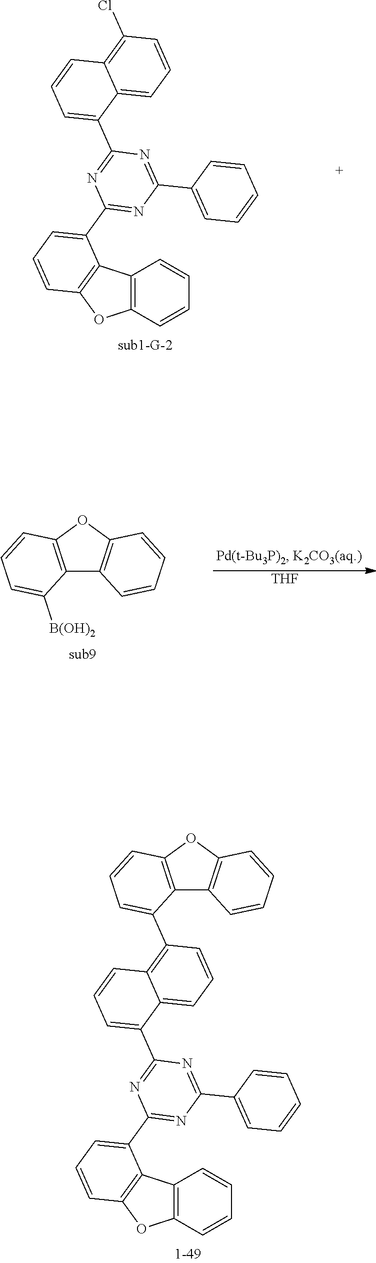 [SMILES: Clc1cccc2c(-c3nc(-c4ccccc4)nc(-c4cccc5oc6ccccc6c45)n3)cccc12 ; OB(O)c1cccc2oc3ccccc3c12 ; "THF" ; c1ccc(-c2nc(-c3cccc4c(-c5cccc6oc7ccccc7c56)cccc34)nc(-c3cccc4oc5ccccc5c34)n2)cc1]

Compound sub1-G-2 (15 g, 31 mmol) and Compound sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, added thereto, and the mixture was sufficiently stirred and then bis(tri-tert-butylphosphine)palladium(0) (0.2 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.7 g of Compound 1-49. (Yield: 72%, MS: [M+H]⁺=616)

### Preparation Example 1-50: Preparation of Compound 1-50

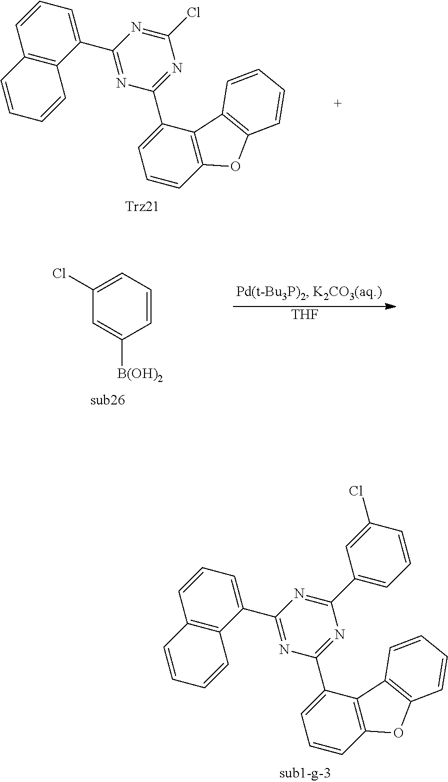 [SMILES: Clc1nc(-c2cccc3ccccc23)nc(-c2cccc3oc4ccccc4c23)n1 ; OB(O)c1cccc(Cl)c1 ; Clc1cccc(-c2nc(-c3cccc4ccccc34)nc(-c3cccc4oc5ccccc5c34)n2)c1 ; "THF"]

Compound Trz21 (15 g, 36.8 mmol) and Compound sub26 (5.8 g, 36.8 mmol) were added to 300 ml of THF under a nitrogen atmosphere, and the mixture was stirred and refluxed. Then, potassium carbonate (15.2 g, 110.3 mmol) was dissolved in 46 ml of water, added thereto, and the mixture was sufficiently stirred and then bis(tri-tert-butylphosphine)palladium(0) (0.2 g, 0.4 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.8 g of Compound sub1-G-3. (Yield: 72%, MS: [M+H]⁺=484)

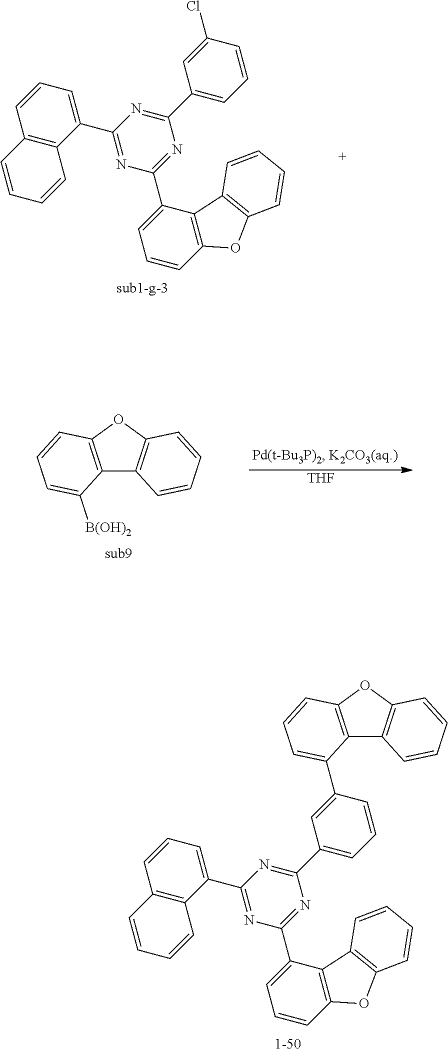 [SMILES: Clc1cccc(-c2nc(-c3cccc4ccccc34)nc(-c3cccc4oc5ccccc5c34)n2)c1 ; OB(O)c1cccc2oc3ccccc3c12 ; "THF" ; c1cc(-c2nc(-c3cccc4ccccc34)nc(-c3cccc4oc5ccccc5c34)n2)cc(-c2cccc3oc4ccccc4c23)c1]

Compound sub1-G-3 (15 g, 31 mmol) and Compound sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.2 g of Compound 1-50. (Yield: 69%, MS: [M+H]⁺=616)

### Preparation Example 1-51: Preparation of Compound 1-51

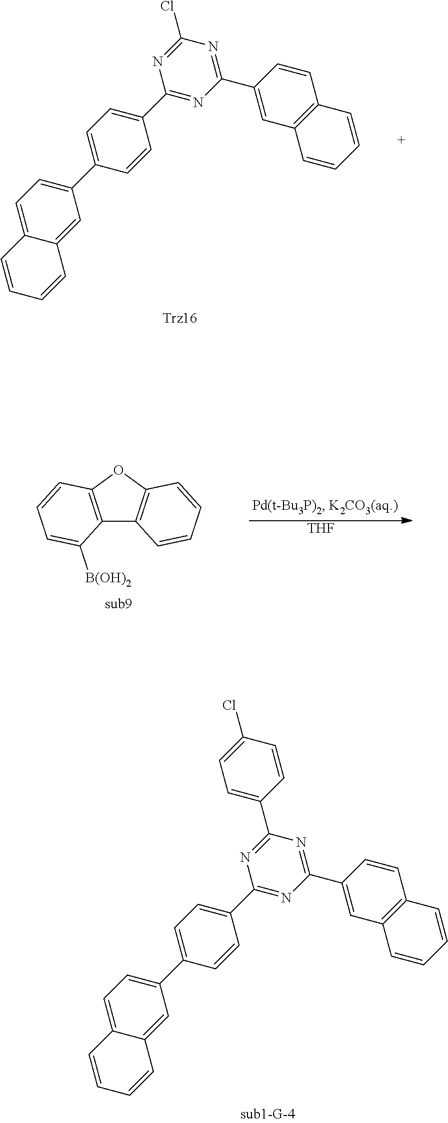 [SMILES: Clc1nc(-c2ccc(-c3ccc4ccccc4c3)cc2)nc(-c2ccc3ccccc3c2)n1 ; OB(O)c1cccc2oc3ccccc3c12 ; Clc1ccc(-c2nc(-c3ccc(-c4ccc5ccccc5c4)cc3)nc(-c3ccc4ccccc4c3)n2)cc1 ; "THF"]

Compound Trz16 (15 g, 36.8 mmol) and Compound sub27 (5.3 g, 33.8 mmol) were added to 300 ml of THF under a nitrogen atmosphere, and the mixture was stirred and refluxed. Then, potassium carbonate (14 g, 101.4 mmol) was dissolved in 42 ml of water, added thereto, and the mixture was sufficiently stirred and then bis(tri-tert-butylphosphine)palladium(0) (0.2 g, 0.3 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.3 g of Compound sub1-G-4. (Yield: 76%, MS: [M+H]⁺=520)

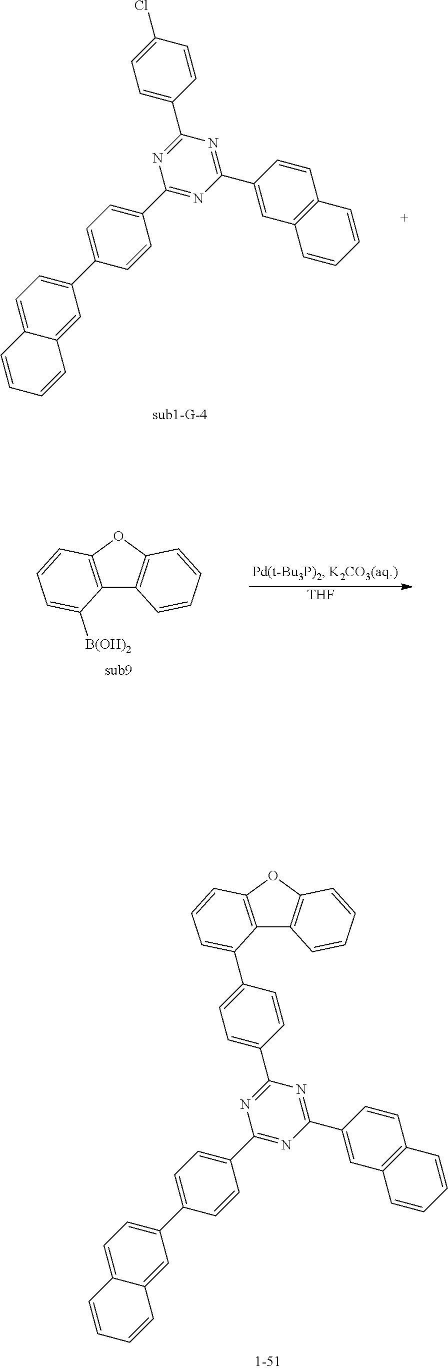 [SMILES: Clc1ccc(-c2nc(-c3ccc(-c4ccc5ccccc5c4)cc3)nc(-c3ccc4ccccc4c3)n2)cc1 ; OB(O)c1cccc2oc3ccccc3c12 ; "THF" ; c1ccc2cc(-c3ccc(-c4nc(-c5ccc(-c6cccc7oc8ccccc8c67)cc5)nc(-c5ccc6ccccc6c5)n4)cc3)ccc2c1]

Compound sub1-G-4 (15 g, 28.8 mmol) and Compound sub9 (6.1 g, 28.8 mmol) were added to 300 ml of THF under a nitrogen atmosphere, and the mixture was stirred and refluxed. Then, potassium carbonate (12 g, 86.5 mmol) was dissolved in 36 ml of water, added thereto, and the mixture was sufficiently stirred and then bis(tri-tert-butylphosphine)palladium(0) (0.1 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.3 g of Compound 1-51. (Yield: 71%, MS: [M+H]⁺=652)

### Preparation Example 1-52: Preparation of Compound 1-52

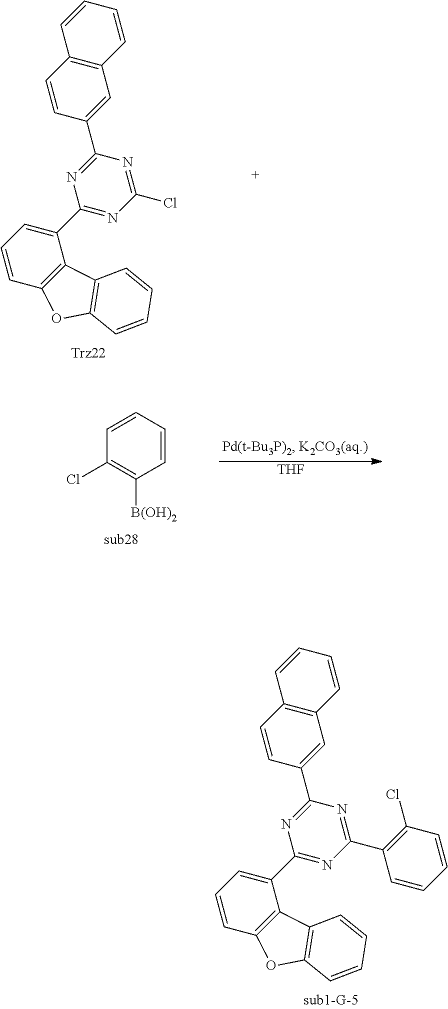 [SMILES: Clc1nc(-c2ccc3ccccc3c2)nc(-c2cccc3oc4ccccc4c23)n1 ; OB(O)c1ccccc1Cl ; Clc1ccccc1-c1nc(-c2ccc3ccccc3c2)nc(-c2cccc3oc4ccccc4c23)n1 ; "THF"]

Compound Trz22 (15 g, 36.8 mmol) and Compound sub28 (5.8 g, 36.8 mmol) were added to 300 ml of THF under a nitrogen atmosphere, and the mixture was stirred and refluxed. Then, potassium carbonate (15.2 g, 110.3 mmol) was dissolved in 46 ml of water, added thereto, and the mixture was sufficiently stirred and then bis(tri-tert-butylphosphine)palladium(0) (0.2 g, 0.4 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.8 g of Compound sub1-G-5. (Yield: 72%, MS: [M+H]⁺=484)

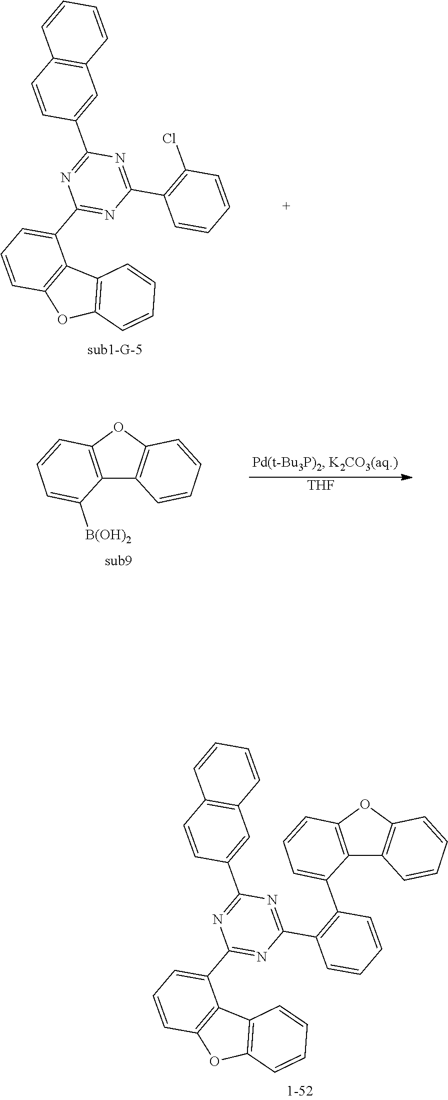 [SMILES: Clc1ccccc1-c1nc(-c2ccc3ccccc3c2)nc(-c2cccc3oc4ccccc4c23)n1 ; OB(O)c1cccc2oc3ccccc3c12 ; "THF" ; c1ccc(-c2cccc3oc4ccccc4c23)c(-c2nc(-c3ccc4ccccc4c3)nc(-c3cccc4oc5ccccc5c34)n2)c1]

Compound sub1-G-5 (15 g, 31 mmol) and Compound sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13 g of Compound 1-52. (Yield: 68%, MS: [M+H]⁺=616)

### Preparation Example 1-53: Preparation of Compound 1-53

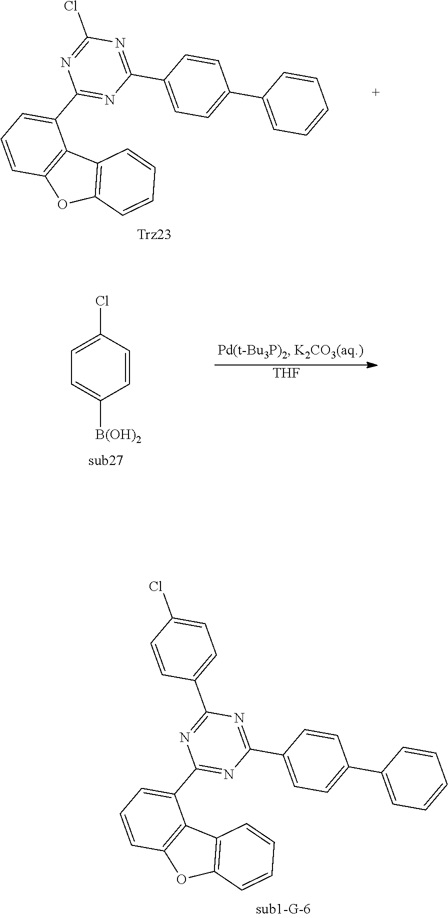 [SMILES: Clc1nc(-c2ccc(-c3ccccc3)cc2)nc(-c2cccc3oc4ccccc4c23)n1 ; OB(O)c1ccc(Cl)cc1 ; "THF" ; Clc1ccc(-c2nc(-c3ccc(-c4ccccc4)cc3)nc(-c3cccc4oc5ccccc5c34)n2)cc1]

Compound Trz23 (15 g, 34.6 mmol) and Compound sub27 (5.4 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 11.3 g of Compound sub1-G-6. (Yield: 64%, MS: [M+H]⁺=510)

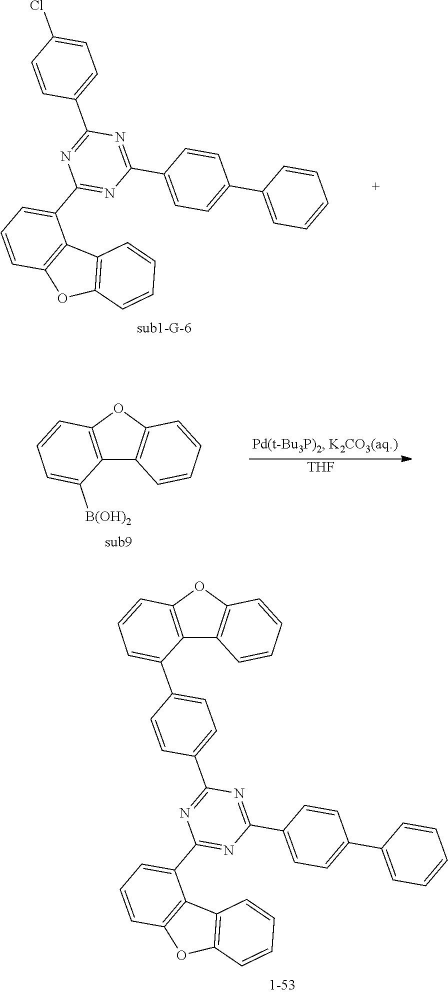 [SMILES: Clc1ccc(-c2nc(-c3ccc(-c4ccccc4)cc3)nc(-c3cccc4oc5ccccc5c34)n2)cc1 ; OB(O)c1cccc2oc3ccccc3c12 ; c1ccc(-c2ccc(-c3nc(-c4ccc(-c5cccc6oc7ccccc7c56)cc4)nc(-c4cccc5oc6ccccc6c45)n3)cc2)cc1 ; "THF"]

Compound sub1-G-6 (15 g, 31 mmol) and Compound sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13 g of Compound 1-53. (Yield: 68%, MS: [M+H]⁺=616)

### Preparation Example 1-54: Preparation of Compound 1-54

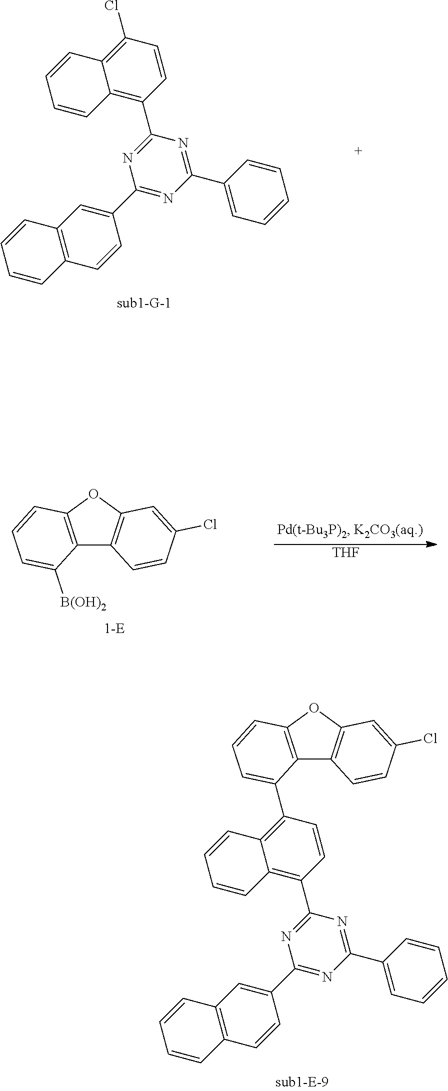 [SMILES: Clc1ccc(-c2nc(-c3ccccc3)nc(-c3ccc4ccccc4c3)n2)c2ccccc12 ; OB(O)c1cccc2oc3cc(Cl)ccc3c12 ; Clc1ccc2c(c1)oc1cccc(-c3ccc(-c4nc(-c5ccccc5)nc(-c5ccc6ccccc6c5)n4)c4ccccc34)c12 ; "THF"]

Compound sub-G-1 (15 g, 33.8 mmol) and Compound 1-E (8.3 g, 33.8 mmol) were added to 300 ml of THF under a nitrogen atmosphere, and the mixture was stirred and refluxed. Then, potassium carbonate (14 g, 101.4 mmol) was dissolved in 42 ml of water, added thereto, and the mixture was sufficiently stirred and then bis(tri-tert-butylphosphine)palladium(0) (0.2 g, 0.3 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.4 g of Compound sub1-E-9. (Yield: 70%, MS: [M+H]⁺=610)

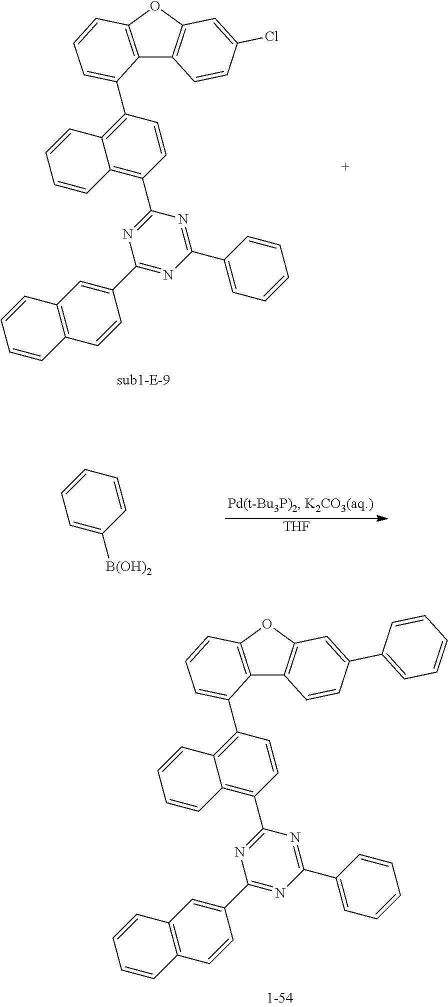 [SMILES: Clc1ccc2c(c1)oc1cccc(-c3ccc(-c4nc(-c5ccccc5)nc(-c5ccc6ccccc6c5)n4)c4ccccc34)c12 ; OB(O)c1ccccc1 ; c1ccc(-c2ccc3c(c2)oc2cccc(-c4ccc(-c5nc(-c6ccccc6)nc(-c6ccc7ccccc7c6)n5)c5ccccc45)c23)cc1 ; "THF"]

Compound sub1-E-9 (15 g, 24.6 mmol) and Compound sub10 (3 g, 24.6 mmol) were added to 300 ml of THF under a nitrogen atmosphere, and the mixture was stirred and refluxed. Then, potassium carbonate (10.2 g, 73.8 mmol) was dissolved in 31 ml of water, added thereto, and the mixture was sufficiently stirred and then bis(tri-tert-butylphosphine)palladium(0) (0.1 g, 0.2 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.2 g of Compound 1-54. (Yield: 76%, MS: [M+H]⁺=652)

### Preparation Example 1-55: Preparation of Compound 1-55

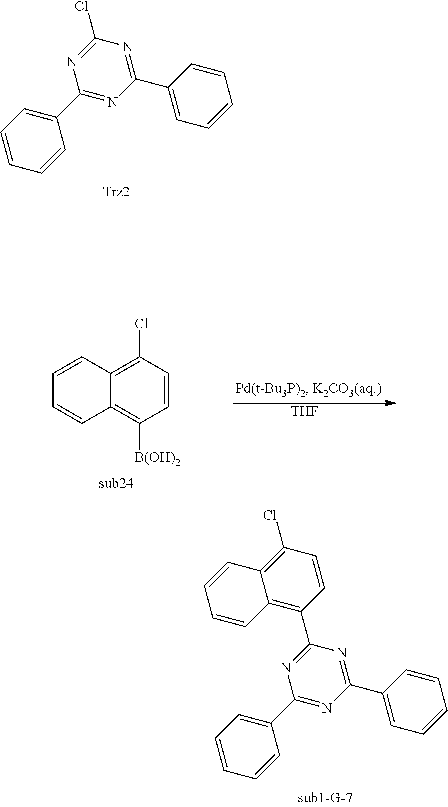 [SMILES: Clc1nc(-c2ccccc2)nc(-c2ccccc2)n1 ; OB(O)c1ccc(Cl)c2ccccc12 ; Clc1ccc(-c2nc(-c3ccccc3)nc(-c3ccccc3)n2)c2ccccc12 ; "THF"]

Compound Trz2 (15 g, 56 mmol) and Compound sub24 (11.6 g, 56 mmol) were added to 300 ml of THE under a nitrogen atmosphere, and the mixture was stirred and refluxed. Then, potassium carbonate (23.2 g, 168.1 mmol) was dissolved in 70 ml of water, added thereto, and the mixture was sufficiently stirred and then bis(tri-tert-butylphosphine)palladium(0) (0.3 g, 0.6 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15.6 g of Compound sub1-G-7. (Yield: 71%, MS: [M+H]⁺=394)

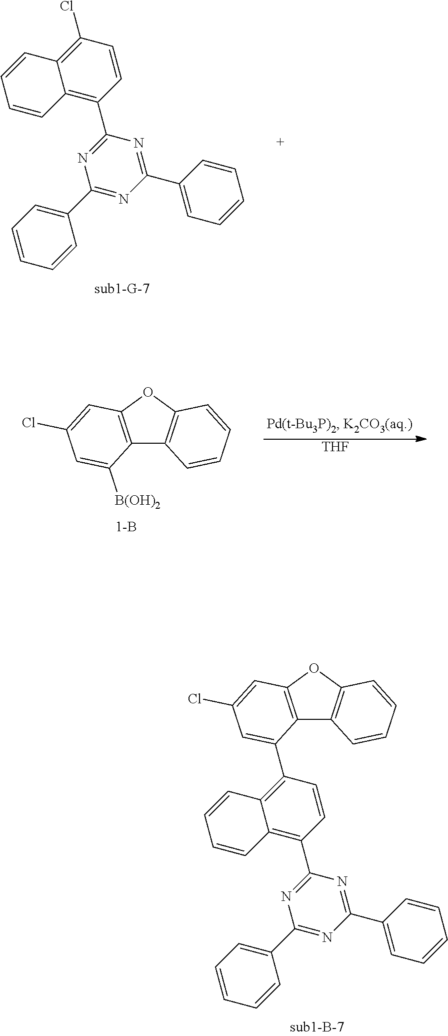 [SMILES: Clc1ccc(-c2nc(-c3ccccc3)nc(-c3ccccc3)n2)c2ccccc12 ; OB(O)c1cc(Cl)cc2oc3ccccc3c12 ; "THF" ; Clc1cc(-c2ccc(-c3nc(-c4ccccc4)nc(-c4ccccc4)n3)c3ccccc23)c2c(c1)oc1ccccc12]

Compound sub1-G-7 (15 g, 38.1 mmol) and Compound 1-B (9.4 g, 38.1 mmol) were added to 300 ml of THF under a nitrogen atmosphere, and the mixture was stirred and refluxed. Then, potassium carbonate (15.8 g, 114.3 mmol) was dissolved in 47 ml of water, added thereto, and the mixture was sufficiently stirred and then bis(tri-tert-butylphosphine)palladium(0) (0.2 g, 0.4 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.8 g of Compound sub1-B-7. (Yield: 65%, MS: [M+H]⁺=560)

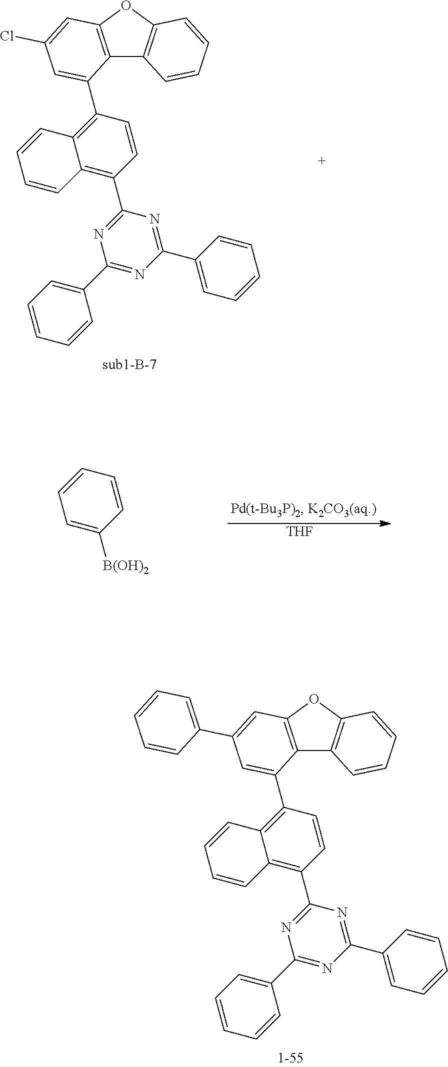 [SMILES: Clc1cc(-c2ccc(-c3nc(-c4ccccc4)nc(-c4ccccc4)n3)c3ccccc23)c2c(c1)oc1ccccc12 ; OB(O)c1ccccc1 ; c1ccc(-c2cc(-c3ccc(-c4nc(-c5ccccc5)nc(-c5ccccc5)n4)c4ccccc34)c3c(c2)oc2ccccc23)cc1 ; "THF"]

Compound sub1-B-7 (15 g, 26.8 mmol) and Compound sub10 (3.3 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, added thereto, and the mixture was sufficiently stirred and then bis(tri-tert-butylphosphine) palladium (0) (0.1 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.9 g of Compound 1-55. (Yield: 80%, MS: [M+H]⁺=602)

### Preparation Example 1-56: Preparation of Compound 1-56

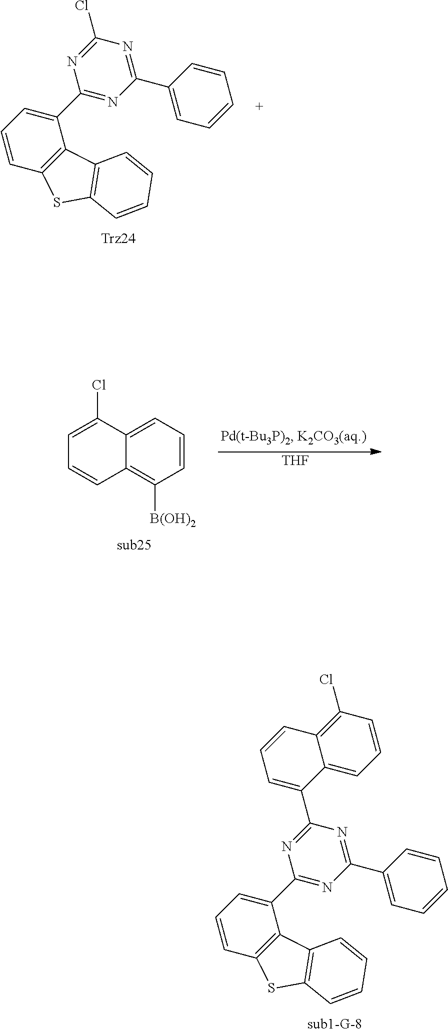 [SMILES: Clc1nc(-c2ccccc2)nc(-c2cccc3sc4ccccc4c23)n1 ; OB(O)c1cccc2c(Cl)cccc12 ; Clc1cccc2c(-c3nc(-c4ccccc4)nc(-c4cccc5sc6ccccc6c45)n3)cccc12 ; "THF"]

Compound Trz24 (15 g, 38.1 mmol) and Compound sub25 (9.4 g, 38.1 mmol) were added to 300 ml of THF under a nitrogen atmosphere, and the mixture was stirred and refluxed. Then, potassium carbonate (15.8 g, 114.3 mmol) was dissolved in 47 ml of water, added thereto, and the mixture was sufficiently stirred and then bis(tri-tert-butylphosphine)palladium(0) (0.2 g, 0.4 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.8 g of Compound sub1-G-8. (Yield: 65%, MS: [M+H]⁺=560)

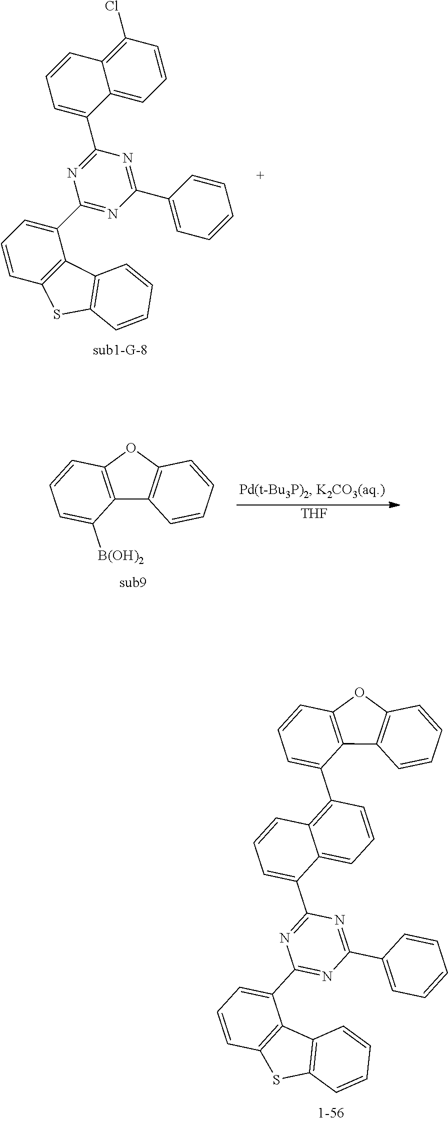 [SMILES: Clc1cccc2c(-c3nc(-c4ccccc4)nc(-c4cccc5sc6ccccc6c45)n3)cccc12 ; OB(O)c1cccc2oc3ccccc3c12 ; c1ccc(-c2nc(-c3cccc4c(-c5cccc6oc7ccccc7c56)cccc34)nc(-c3cccc4sc5ccccc5c34)n2)cc1 ; "THF"]

Compound sub1-G-8 (15 g, 30 mmol) and Compound sub9 (6.4 g, 30 mmol) were added to 300 ml of THF under a nitrogen atmosphere, and the mixture was stirred and refluxed. Then, potassium carbonate (12.4 g, 90 mmol) was dissolved in 37 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.4 g of Compound 1-56. (Yield: 71%, MS: [M+H]⁺=632)

### Preparation Example 1-57: Preparation of Compound 1-57

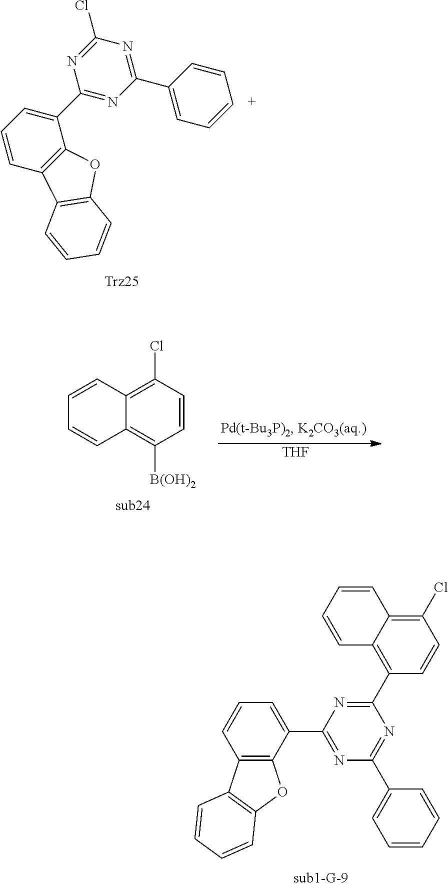 [SMILES: Clc1nc(-c2ccccc2)nc(-c2cccc3c2oc2ccccc23)n1 ; OB(O)c1ccc(Cl)c2ccccc12 ; Clc1ccc(-c2nc(-c3ccccc3)nc(-c3cccc4c3oc3ccccc34)n2)c2ccccc12 ; "THF"]

Compound Trz25 (15 g, 41.9 mmol) and Compound sub24 (8.7 g, 41.9 mmol) were added to 300 ml of THF under a nitrogen atmosphere, and the mixture was stirred and refluxed. Then, potassium carbonate (17.4 g, 125.8 mmol) was dissolved in 52 ml of water, added thereto, and the mixture was sufficiently stirred and then bis(tri-tert-butylphosphine)palladium(0) (0.2 g, 0.4 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.4 g of Compound sub1-G-9. (Yield: 61%, MS: [M+H]⁺=484)

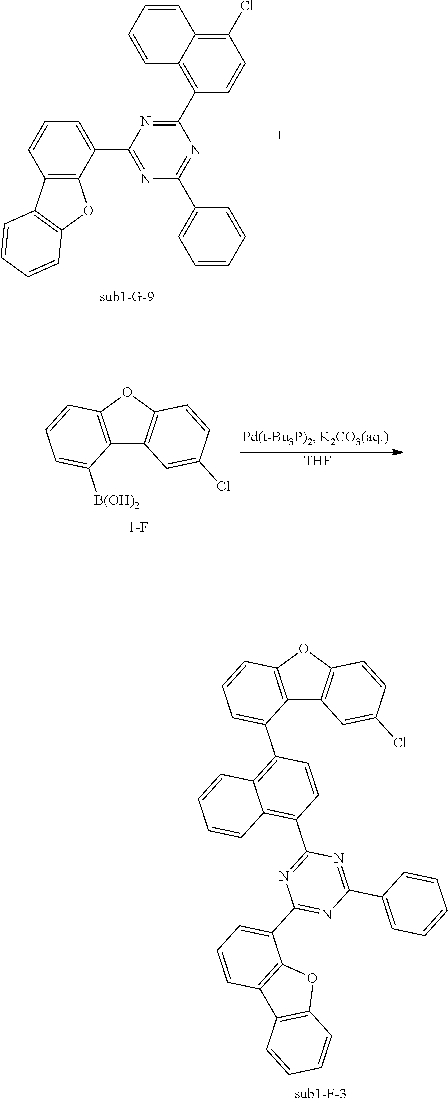 [SMILES: Clc1ccc(-c2nc(-c3ccccc3)nc(-c3cccc4c3oc3ccccc34)n2)c2ccccc12 ; OB(O)c1cccc2oc3ccc(Cl)cc3c12 ; Clc1ccc2oc3cccc(-c4ccc(-c5nc(-c6ccccc6)nc(-c6cccc7c6oc6ccccc67)n5)c5ccccc45)c3c2c1 ; "THF"]

Compound sub1-G-9 (15 g, 31 mmol) and Compound 1-F (7.6 g, 31 mmol) were added to 300 ml of THE under a nitrogen atmosphere, and the mixture was stirred and refluxed. Then, potassium carbonate (12.9 g, 93 mmol) was dissolved in 39 ml of water, added thereto, and the mixture was sufficiently stirred and then bis(tri-tert-butylphosphine)palladium(0) (0.2 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.5 g of Compound sub1-F-3. (Yield: 62%, MS: [M+H]⁺=650)

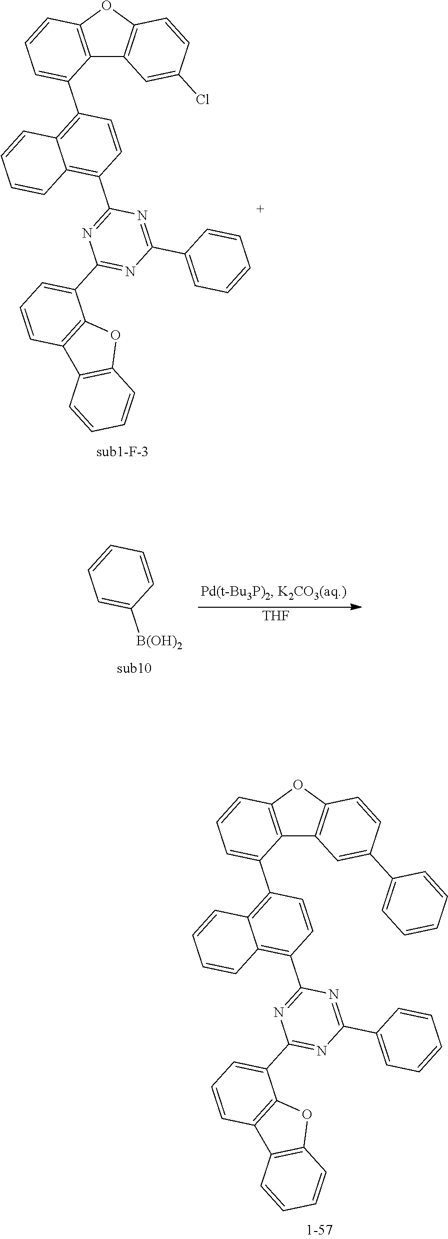 [SMILES: Clc1ccc2oc3cccc(-c4ccc(-c5nc(-c6ccccc6)nc(-c6cccc7c6oc6ccccc67)n5)c5ccccc45)c3c2c1 ; OB(O)c1ccccc1 ; c1ccc(-c2ccc3oc4cccc(-c5ccc(-c6nc(-c7ccccc7)nc(-c7cccc8c7oc7ccccc78)n6)c6ccccc56)c4c3c2)cc1 ; "THF"]

Compound sub1-F-3 (15 g, 23.1 mmol) and Compound sub10 (2.8 g, 23.1 mmol) were added to 300 ml of THF under a nitrogen atmosphere, and the mixture was stirred and refluxed. Then, potassium carbonate (9.6 g, 69.2 mmol) was dissolved in 29 ml of water, added thereto, and the mixture was sufficiently stirred and then bis(tri-tert-butylphosphine)palladium(0) (0.1 g, 0.2 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.8 g of Compound 1-57. (Yield: 80%, MS: [M+H]⁺=692)

### Preparation Example 1-58: Preparation of Compound 1-58

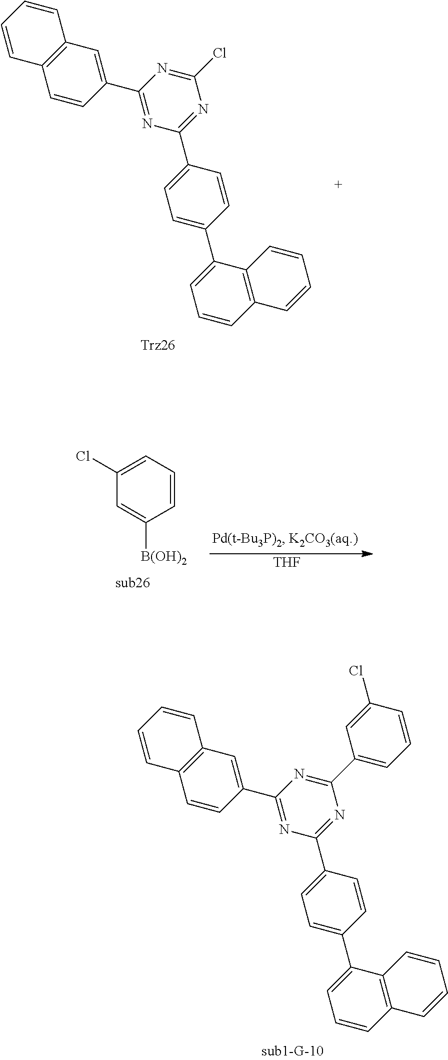 [SMILES: Clc1nc(-c2ccc(-c3cccc4ccccc34)cc2)nc(-c2ccc3ccccc3c2)n1 ; OB(O)c1cccc(Cl)c1 ; Clc1cccc(-c2nc(-c3ccc(-c4cccc5ccccc45)cc3)nc(-c3ccc4ccccc4c3)n2)c1 ; "THF"]

Compound Trz26 (15 g, 33.8 mmol) and Compound sub26 (5.3 g, 33.8 mmol) were added to 300 ml of THF under a nitrogen atmosphere, and the mixture was stirred and refluxed. Then, potassium carbonate (14 g, 101.4 mmol) was dissolved in 42 ml of water, added thereto, and the mixture was sufficiently stirred and then bis(tri-tert-butylphosphine)palladium(0) (0.2 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.5 g of Compound sub1-G-10. (Yield: 60%, MS: [M+H]⁺=520)

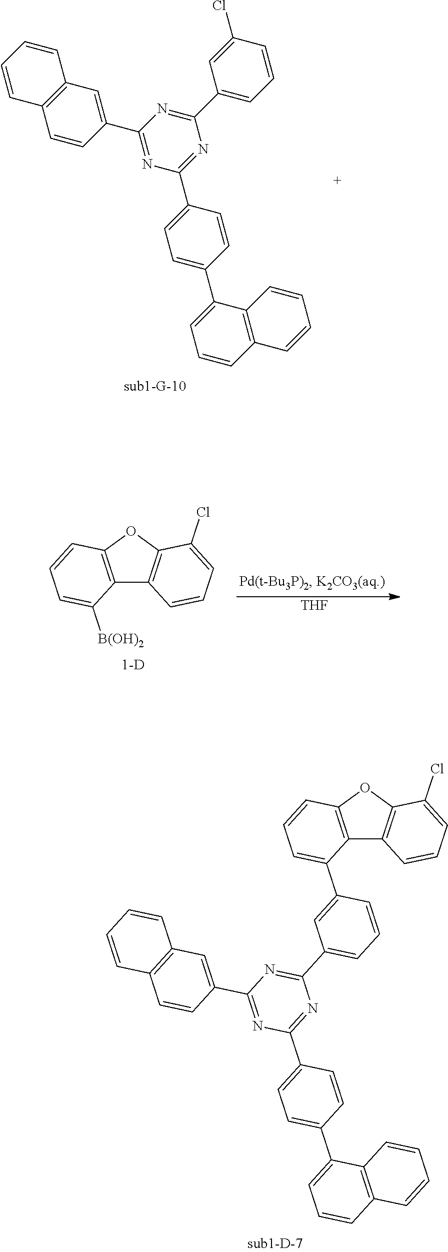 [SMILES: Clc1cccc(-c2nc(-c3ccc(-c4cccc5ccccc45)cc3)nc(-c3ccc4ccccc4c3)n2)c1 ; OB(O)c1cccc2oc3c(Cl)cccc3c12 ; Clc1cccc2c1oc1cccc(-c3cccc(-c4nc(-c5ccc(-c6cccc7ccccc67)cc5)nc(-c5ccc6ccccc6c5)n4)c3)c12 ; "THF"]

Compound sub1-G-10 (15 g, 28.8 mmol) and Compound 1-D (7.1 g, 28.8 mmol) were added to 300 ml of THF under a nitrogen atmosphere, and the mixture was stirred and refluxed. Then, potassium carbonate (12 g, 86.5 mmol) was dissolved in 36 ml of water, added thereto, and the mixture was sufficiently stirred and then bis(tri-tert-butylphosphine)palladium(0) (0.1 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15 g of Compound sub1-D-7. (Yield: 76%, MS: [M+H]⁺=686)

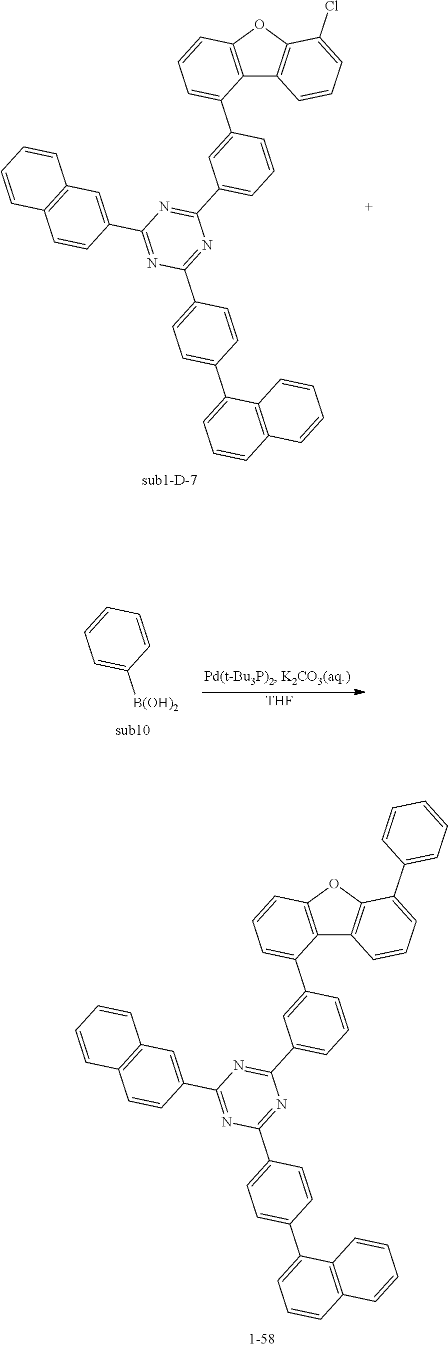 [SMILES: Clc1cccc2c1oc1cccc(-c3cccc(-c4nc(-c5ccc(-c6cccc7ccccc67)cc5)nc(-c5ccc6ccccc6c5)n4)c3)c12 ; OB(O)c1ccccc1 ; c1ccc(-c2cccc3c2oc2cccc(-c4cccc(-c5nc(-c6ccc(-c7cccc8ccccc78)cc6)nc(-c6ccc7ccccc7c6)n5)c4)c23)cc1 ; "THF"]

Compound sub1-D-7 (15 g, 21.9 mmol) and Compound sub10 (2.7 g, 21.9 mmol) were added to 300 ml of THF under a nitrogen atmosphere, and the mixture was stirred and refluxed. Then, potassium carbonate (9.1 g, 65.6 mmol) was dissolved in 27 ml of water, added thereto, and the mixture was sufficiently stirred and then bis(tri-tert-butylphosphine)palladium(0) (0.1 g, 0.2 mmol) was added. After 12-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 9.9 g of Compound 1-58. (Yield: 62%, MS: [M+H]⁺=728)

### Preparation Example 1-59: Preparation of Compound 1-59

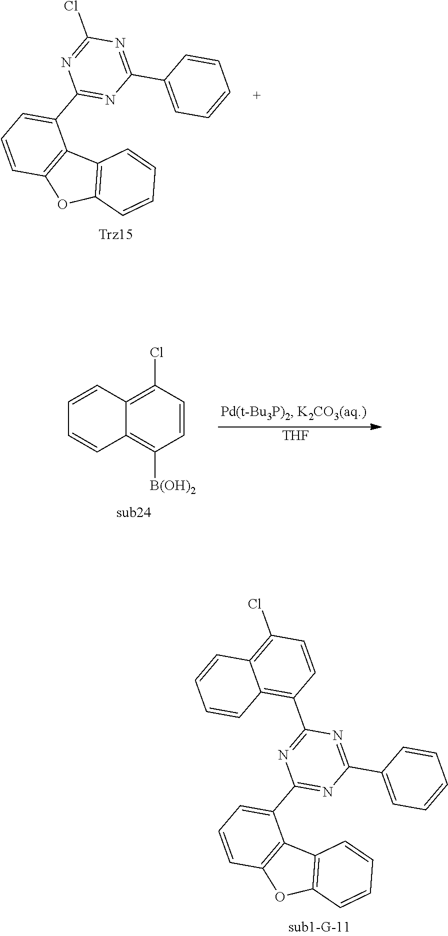 [SMILES: Clc1nc(-c2ccccc2)nc(-c2cccc3oc4ccccc4c23)n1 ; OB(O)c1ccc(Cl)c2ccccc12 ; Clc1ccc(-c2nc(-c3ccccc3)nc(-c3cccc4oc5ccccc5c34)n2)c2ccccc12 ; "THF"]

Compound Trz15 (15 g, 41.9 mmol) and Compound sub24 (8.7 g, 41.9 mmol) were added to 300 ml of THF under a nitrogen atmosphere, and the mixture was stirred and refluxed. Then, potassium carbonate (17.4 g, 125.8 mmol) was dissolved in 52 ml of water, added thereto, and the mixture was sufficiently stirred and then bis(tri-tert-butylphosphine)palladium(0) (0.2 g, 0.4 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.4 g of Compound sub1-G-11. (Yield: 61%, MS: [M+H]⁺=484)

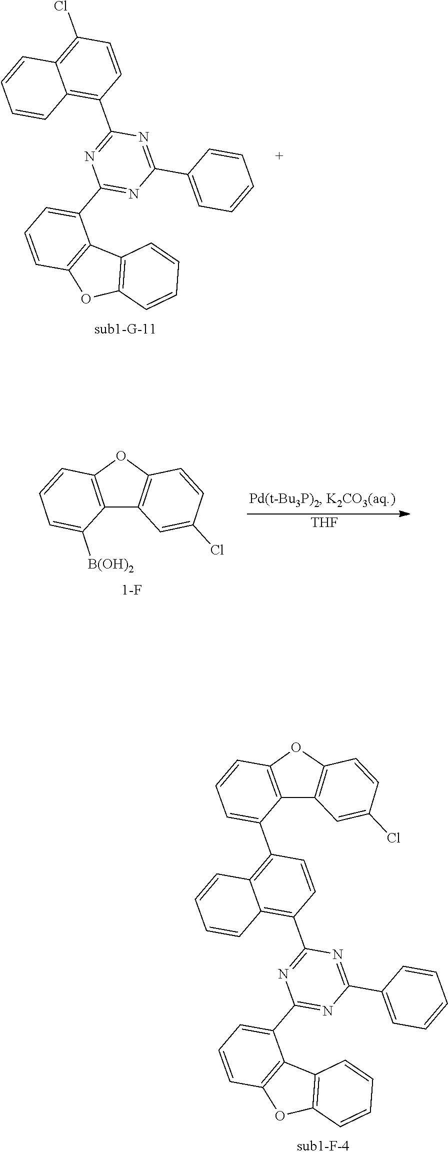 [SMILES: Clc1ccc(-c2nc(-c3ccccc3)nc(-c3cccc4oc5ccccc5c34)n2)c2ccccc12 ; OB(O)c1cccc2oc3ccc(Cl)cc3c12 ; Clc1ccc2oc3cccc(-c4ccc(-c5nc(-c6ccccc6)nc(-c6cccc7oc8ccccc8c67)n5)c5ccccc45)c3c2c1 ; "THF"]

Compound sub1-G-11 (15 g, 28.8 mmol) and Compound 1-F (7.1 g, 28.8 mmol) were added to 300 ml of THF under a nitrogen atmosphere, and the mixture was stirred and refluxed. Then, potassium carbonate (12 g, 86.5 mmol) was dissolved in 36 ml of water, added thereto, and the mixture was sufficiently stirred and then bis(tri-tert-butylphosphine)palladium(0) (0.1 g, 0.3 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 15 g of Compound sub1-F-4. (Yield: 76%, MS: [M+H]⁺=686)

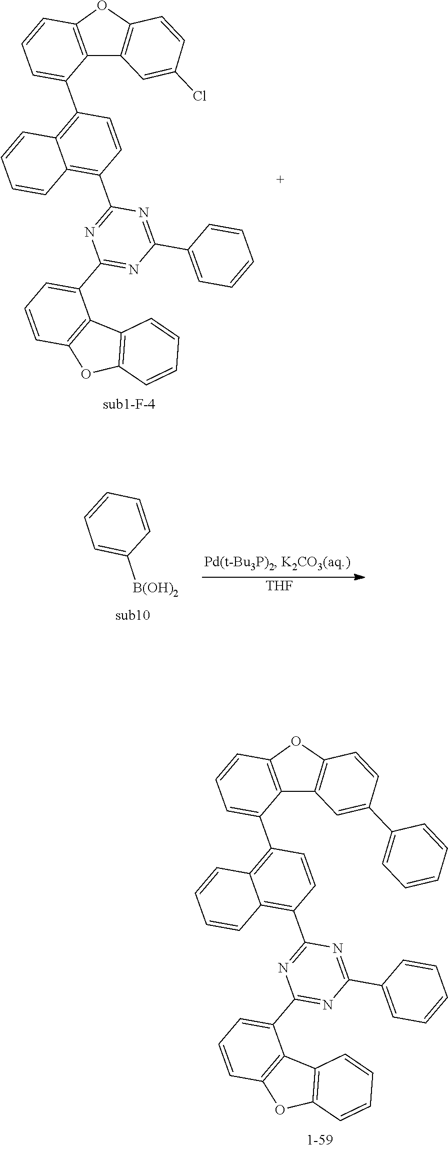 [SMILES: Clc1ccc2oc3cccc(-c4ccc(-c5nc(-c6ccccc6)nc(-c6cccc7oc8ccccc8c67)n5)c5ccccc45)c3c2c1 ; OB(O)c1ccccc1 ; c1ccc(-c2ccc3oc4cccc(-c5ccc(-c6nc(-c7ccccc7)nc(-c7cccc8oc9ccccc9c78)n6)c6ccccc56)c4c3c2)cc1 ; "THF"]

Compound sub1-F-4 (15 g, 23.1 mmol) and Compound sub10 (2.8 g, 23.1 mmol) were added to 300 ml of THF under a nitrogen atmosphere, and the mixture was stirred and refluxed. Then, potassium carbonate (9.6 g, 69.2 mmol) was dissolved in 29 ml of water, added thereto, and the mixture was sufficiently stirred and then bis(tri-tert-butylphosphine)palladium(0) (0.1 g, 0.2 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.1 g of Compound 1-59. (Yield: 76%, MS: [M+H]⁺=692)

### Preparation Example 1-60: Preparation of Compound 1-60

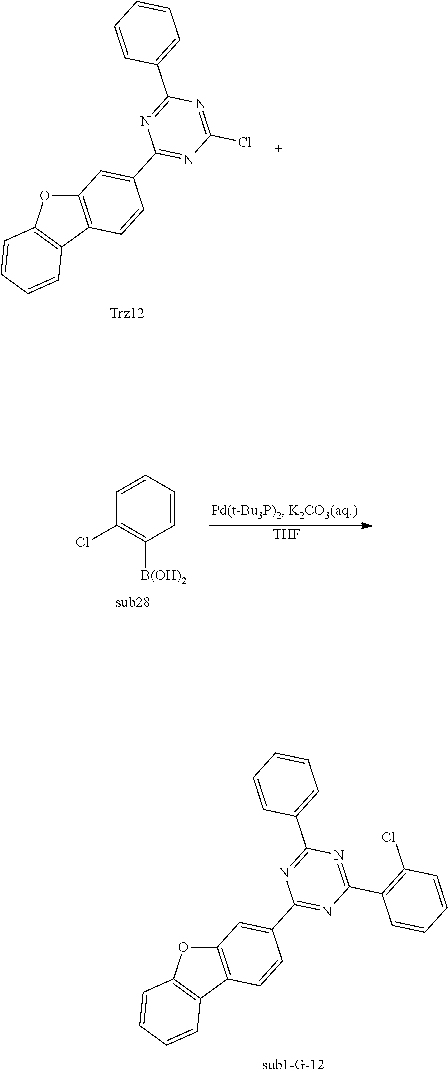 [SMILES: Clc1nc(-c2ccccc2)nc(-c2ccc3c(c2)oc2ccccc23)n1 ; OB(O)c1ccccc1Cl ; Clc1ccccc1-c1nc(-c2ccccc2)nc(-c2ccc3c(c2)oc2ccccc23)n1 ; "THF"]

Compound Trz12 (15 g, 41.9 mmol) and Compound sub28 (6.6 g, 41.9 mmol) were added to 300 ml of THF under a nitrogen atmosphere, and the mixture was stirred and refluxed. Then, potassium carbonate (17.4 g, 125.8 mmol) was dissolved in 52 ml of water, added thereto, and the mixture was sufficiently stirred and then bis(tri-tert-butylphosphine)palladium(0) (0.2 g, 0.4 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 11.1 g of Compound sub1-G-12. (Yield: 61%, MS: [M+H]⁺=434)

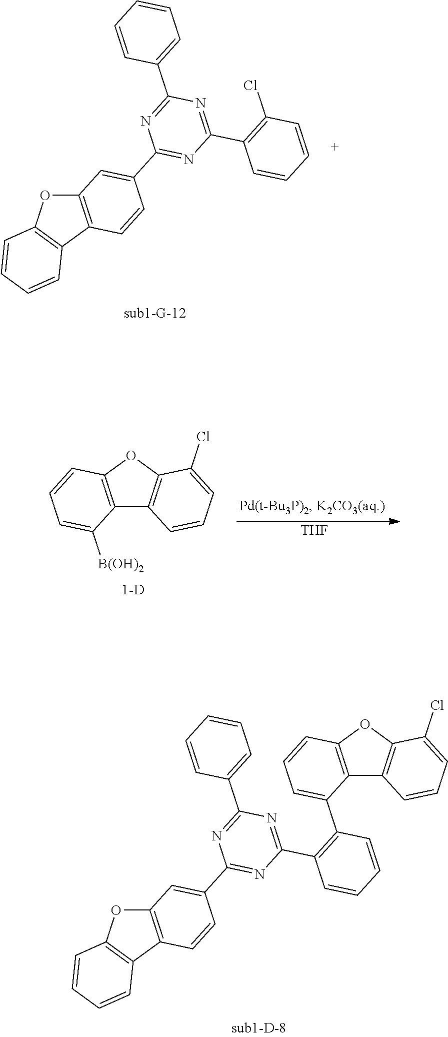 [SMILES: Clc1ccccc1-c1nc(-c2ccccc2)nc(-c2ccc3c(c2)oc2ccccc23)n1 ; OB(O)c1cccc2oc3c(Cl)cccc3c12 ; Clc1cccc2c1oc1cccc(-c3ccccc3-c3nc(-c4ccccc4)nc(-c4ccc5c(c4)oc4ccccc45)n3)c12 ; "THF"]

Compound sub1-G-12 (15 g, 34.6 mmol) and Compound 1-D (8.5 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, added thereto, and the mixture was sufficiently stirred and then bis(tri-tert-butylphosphine)palladium(0) (0.2 g, 0.3 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.6 g of Compound sub1-D-8. (Yield: 79%, MS: [M+H]⁺=500)

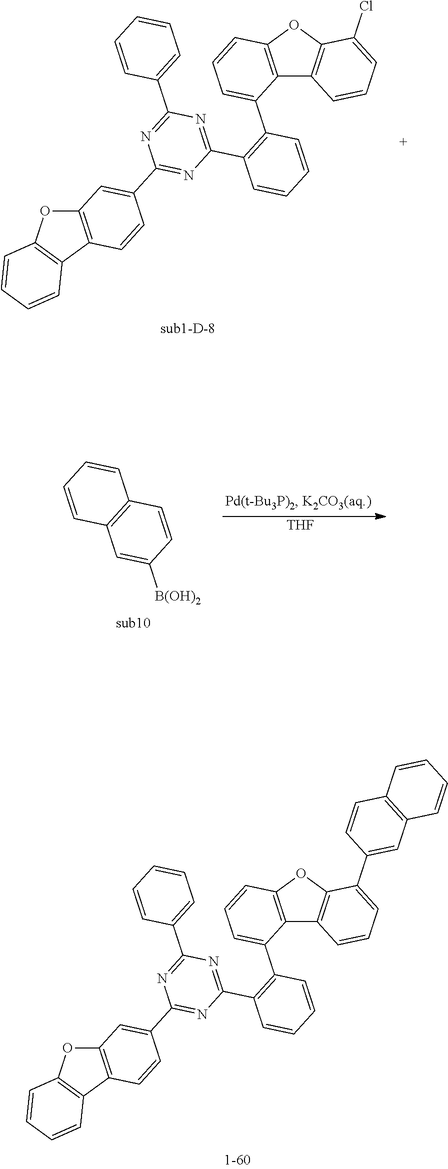 [SMILES: Clc1cccc2c1oc1cccc(-c3ccccc3-c3nc(-c4ccccc4)nc(-c4ccc5c(c4)oc4ccccc45)n3)c12 ; OB(O)c1ccc2ccccc2c1 ; "THF" ; c1ccc(-c2nc(-c3ccc4c(c3)oc3ccccc34)nc(-c3ccccc3-c3cccc4oc5c(-c6ccc7ccccc7c6)cccc5c34)n2)cc1]

Compound sub1-D-8 (15 g, 25 mmol) and Compound sub10 (4.3 g, 25 mmol) were added to 300 ml of THF under a nitrogen atmosphere, and the mixture was stirred and refluxed. Then, potassium carbonate (10.4 g, 75 mmol) was dissolved in 31 ml of water, added thereto, and the mixture was sufficiently stirred and then bis(tri-tert-butylphosphine)palladium(0) (0.1 g, 0.2 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 13.3 g of Compound 1-60. (Yield: 77%, MS: [M+H]⁺=692)

### Preparation Example 2-1: Preparation of Compound 2-1

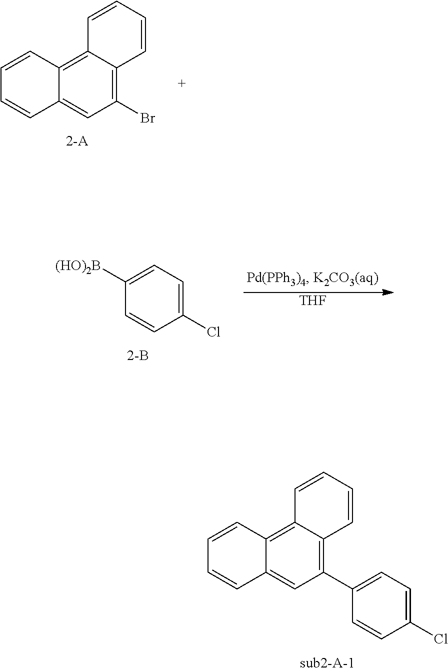 [SMILES: Brc1cc2ccccc2c2ccccc12 ; OB(O)c1ccc(Cl)cc1 ; Clc1ccc(-c2cc3ccccc3c3ccccc23)cc1 ; "THF"]

Compound 2-A (15 g, 58.3 mmol) and Compound 2-B (10 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water, added thereto, and the mixture was sufficiently stirred and then tetrakis(triphenylphosphine)palladium(0)(1.3 g, 1.2 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.6 g of Compound sub2-A-1. (Yield: 75%, MS: [M+H]⁺=289)

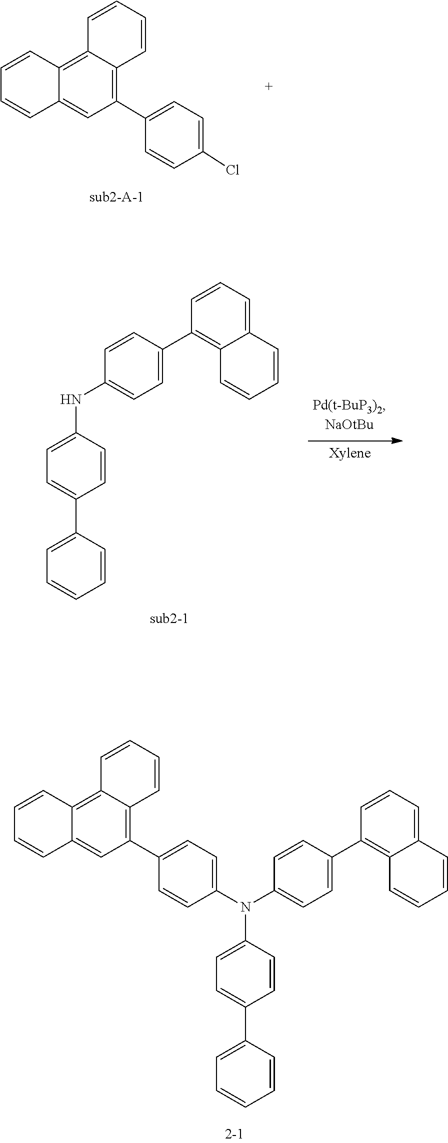 [SMILES: Clc1ccc(-c2cc3ccccc3c3ccccc23)cc1 ; c1ccc(-c2ccc(Nc3ccc(-c4cccc5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1]

Compound sub2-A-1 (10 g, 34.6 mmol), Compound sub2-1 (12.9 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.7 g of Compound 2-1. (Yield: 59%, MS: [M+H]⁺=624)

### Preparation Example 2-2: Preparation of Compound 2-2

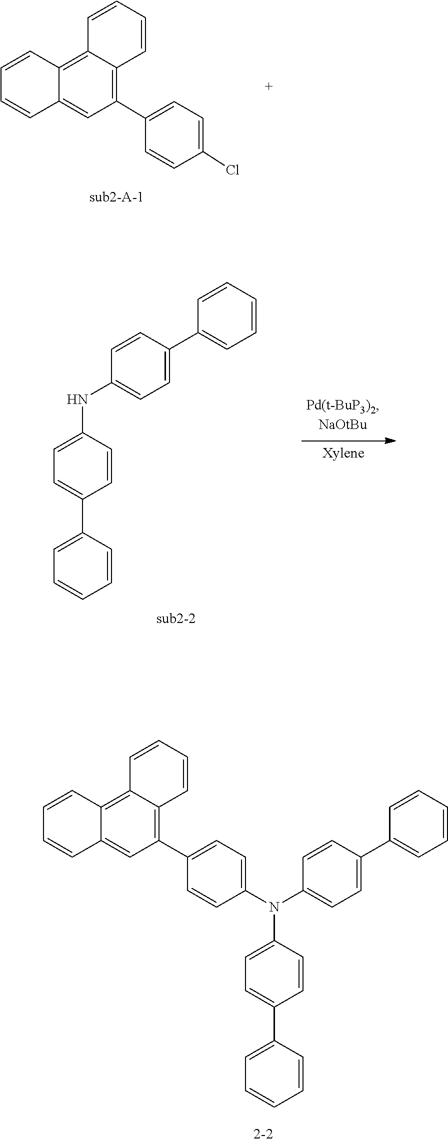 [SMILES: Clc1ccc(-c2cc3ccccc3c3ccccc23)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4cc5ccccc5c5ccccc45)cc3)cc2)cc1]

Compound sub2-A-1 (10 g, 34.6 mmol), Compound sub2-2 (11.1 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 10.1 g of Compound 2-2. (Yield: 51%, MS: [M+H]⁺=574)

### Preparation Example 2-3: Preparation of Compound 2-3

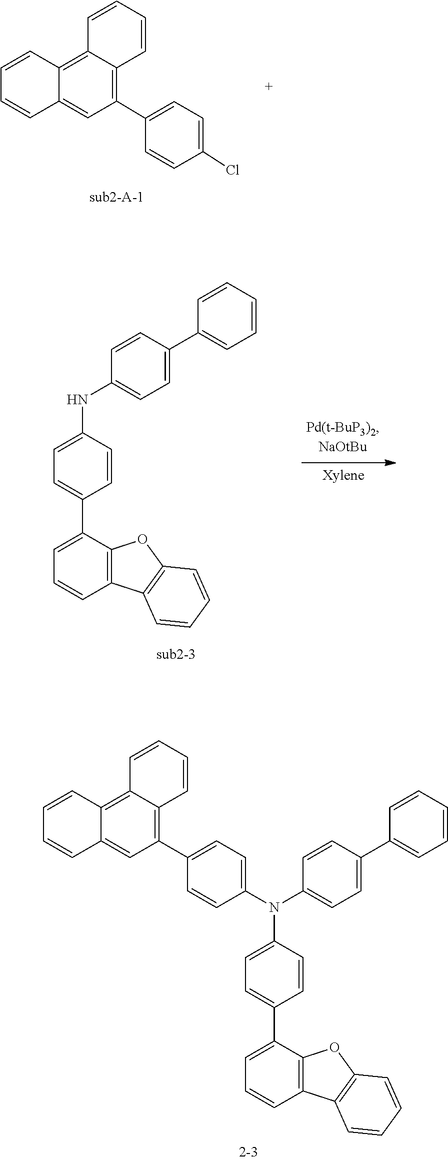 [SMILES: Clc1ccc(-c2cc3ccccc3c3ccccc23)cc1 ; c1ccc(-c2ccc(Nc3ccc(-c4cccc5c4oc4ccccc45)cc3)cc2)cc1 ; c1ccc(-c2ccc(N(c3ccc(-c4cc5ccccc5c5ccccc45)cc3)c3ccc(-c4cccc5c4oc4ccccc45)cc3)cc2)cc1]

Compound sub2-A-1 (10 g, 34.6 mmol), Compound sub2-3 (14.3 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.2 g of Compound 2-3. (Yield: 53%, MS: [M+H]⁺=664)

### Preparation Example 2-4: Preparation of Compound 2-4

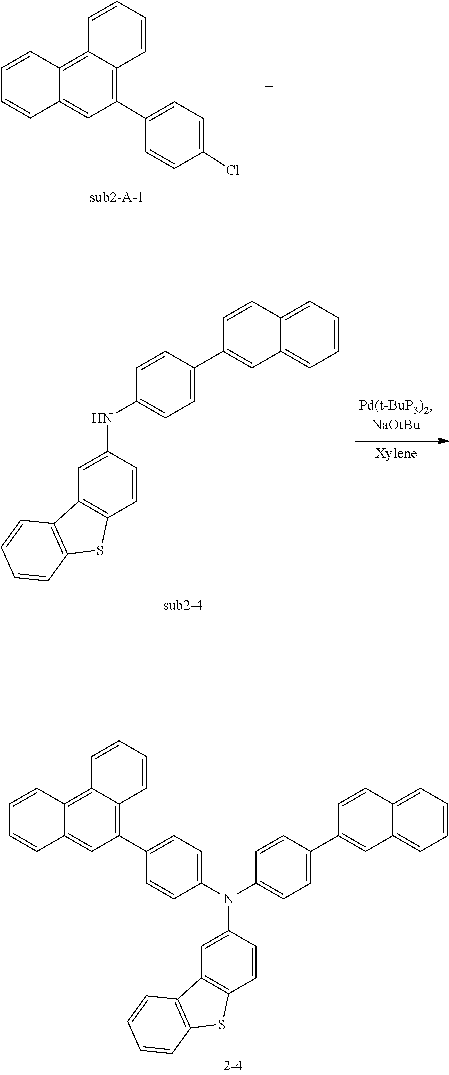 [SMILES: Clc1ccc(-c2cc3ccccc3c3ccccc23)cc1 ; c1ccc2cc(-c3ccc(Nc4ccc5sc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1]

Compound sub2-A-1 (10 g, 34.6 mmol), Compound sub2-4 (13.9 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 14 g of Compound 2-4. (Yield: 62%, MS: [M+H]⁺=654)

### Preparation Example 2-5: Preparation of Compound 2-5

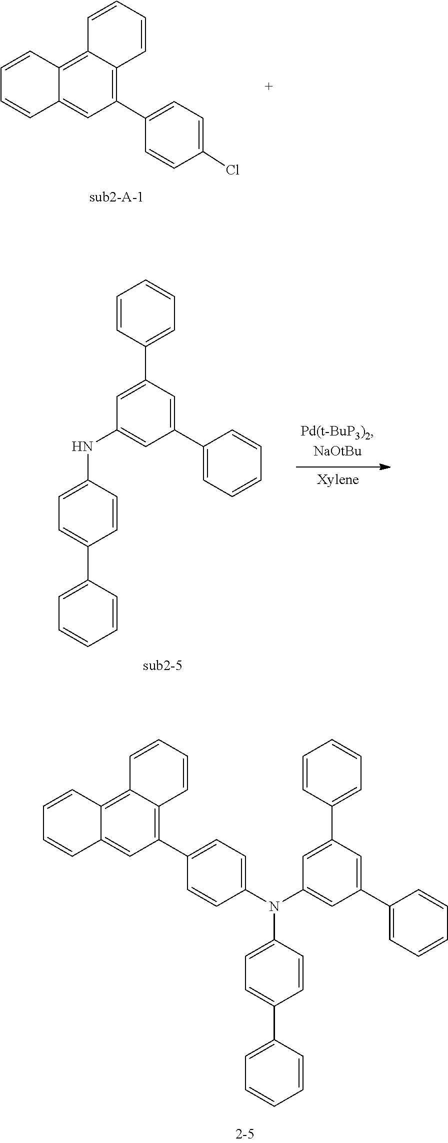 [SMILES: Clc1ccc(-c2cc3ccccc3c3ccccc23)cc1 ; c1ccc(-c2ccc(Nc3cc(-c4ccccc4)cc(-c4ccccc4)c3)cc2)cc1 ; c1ccc(-c2ccc(N(c3ccc(-c4cc5ccccc5c5ccccc45)cc3)c3cc(-c4ccccc4)cc(-c4ccccc4)c3)cc2)cc1]

Compound sub2-A-1 (10 g, 34.6 mmol), Compound sub2-5 (13.8 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.2 g of Compound 2-5. (Yield: 50%, MS: [M+H]⁺=650)

### Preparation Example 2-6: Preparation of Compound 2-6

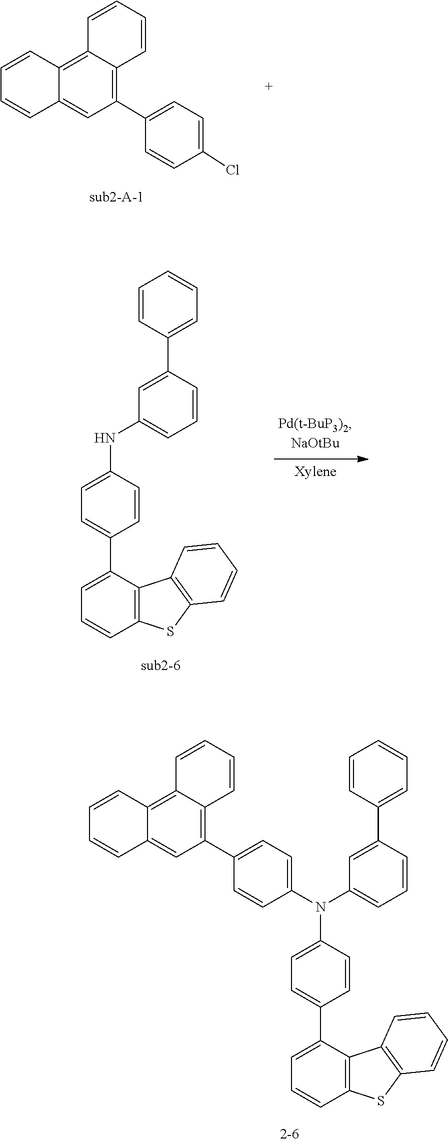 [SMILES: Clc1ccc(-c2cc3ccccc3c3ccccc23)cc1 ; c1ccc(-c2cccc(Nc3ccc(-c4cccc5sc6ccccc6c45)cc3)c2)cc1 ; c1ccc(-c2cccc(N(c3ccc(-c4cc5ccccc5c5ccccc45)cc3)c3ccc(-c4cccc5sc6ccccc6c45)cc3)c2)cc1]

Compound sub2-A-1 (10 g, 34.6 mmol), Compound sub2-6 (14.8 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.2 g of Compound 2-6. (Yield: 52%, MS: [M+H]⁺=680)

### Preparation Example 2-7: Preparation of Compound 2-7

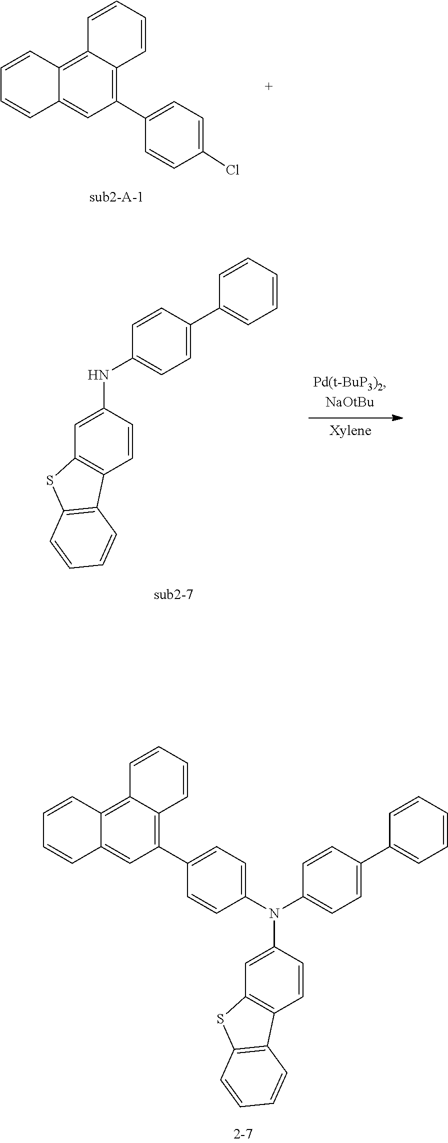 [SMILES: Clc1ccc(-c2cc3ccccc3c3ccccc23)cc1 ; c1ccc(-c2ccc(Nc3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2)cc1]

Compound sub2-A-1 (10 g, 34.6 mmol), Compound sub2-7 (12.2 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 1 g of Compound 2-7. (Yield: 50%, MS: [M+H]⁺=61)

### Preparation Example 2-8: Preparation of Compound 2-8

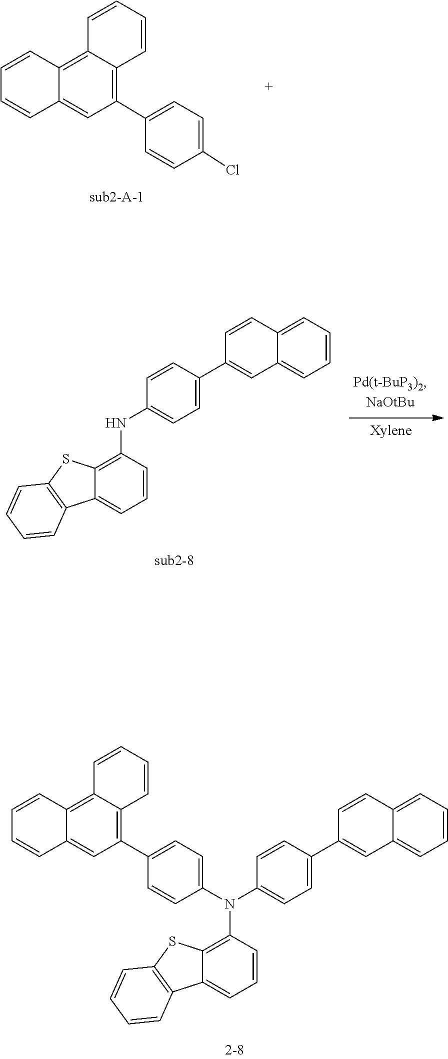 [SMILES: Clc1ccc(-c2cc3ccccc3c3ccccc23)cc1 ; c1ccc2cc(-c3ccc(Nc4cccc5c4sc4ccccc45)cc3)ccc2c1 ; c1ccc2cc(-c3ccc(N(c4ccc(-c5cc6ccccc6c6ccccc56)cc4)c4cccc5c4sc4ccccc45)cc3)ccc2c1]

Compound sub2-A-1 (10 g, 34.6 mmol), Compound sub2-8 (13.9 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 13.3 g of Compound 2-8. (Yield: 59%, MS: [M+H]⁺=654)

### Preparation Example 2-9: Preparation of Compound 2-9

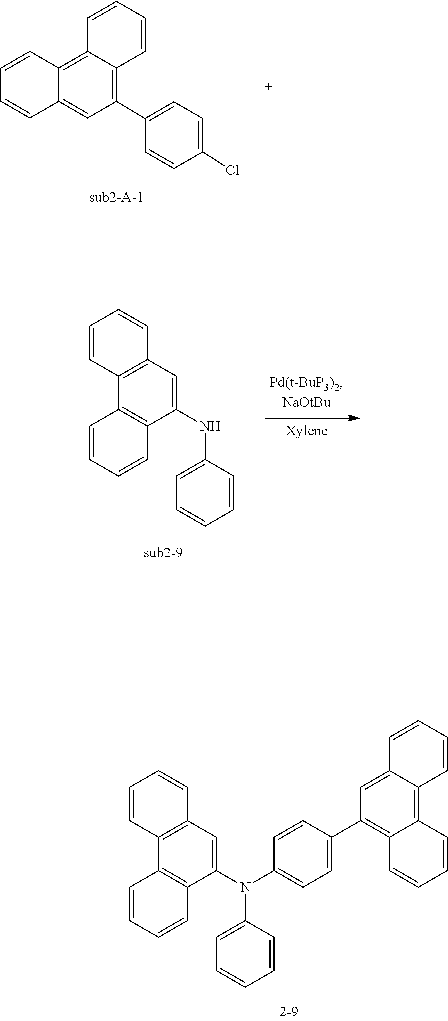 [SMILES: Clc1ccc(-c2cc3ccccc3c3ccccc23)cc1 ; c1ccc(Nc2cc3ccccc3c3ccccc23)cc1 ; c1ccc(N(c2ccc(-c3cc4ccccc4c4ccccc34)cc2)c2cc3ccccc3c3ccccc23)cc1]

Compound sub2-A-1 (10 g, 34.6 mmol), Compound sub2-9 (9.3 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.2 g of Compound 2-9. (Yield: 62%, MS: [M+H]⁺=522)

### Preparation Example 2-10: Preparation of Compound 2-10

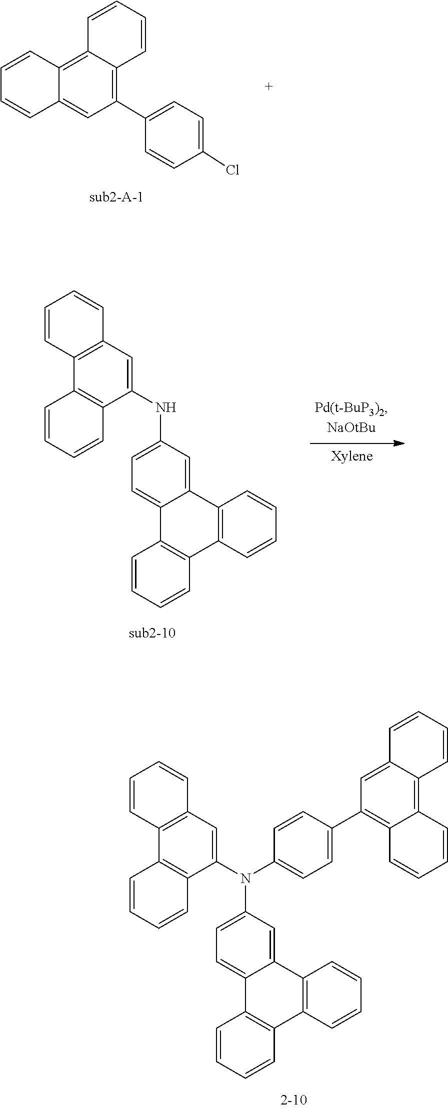 [SMILES: Clc1ccc(-c2cc3ccccc3c3ccccc23)cc1 ; c1ccc2c(c1)cc(Nc1ccc3c4ccccc4c4ccccc4c3c1)c1ccccc12 ; c1ccc2c(c1)cc(-c1ccc(N(c3ccc4c5ccccc5c5ccccc5c4c3)c3cc4ccccc4c4ccccc34)cc1)c1ccccc12]

Compound sub2-A-1 (10 g, 34.6 mmol), Compound sub2-10 (14.5 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 14.4 g of Compound 2-10. (Yield: 62%, MS: [M+H]⁺=572)

### Preparation Example 2-11: Preparation of Compound 2-11

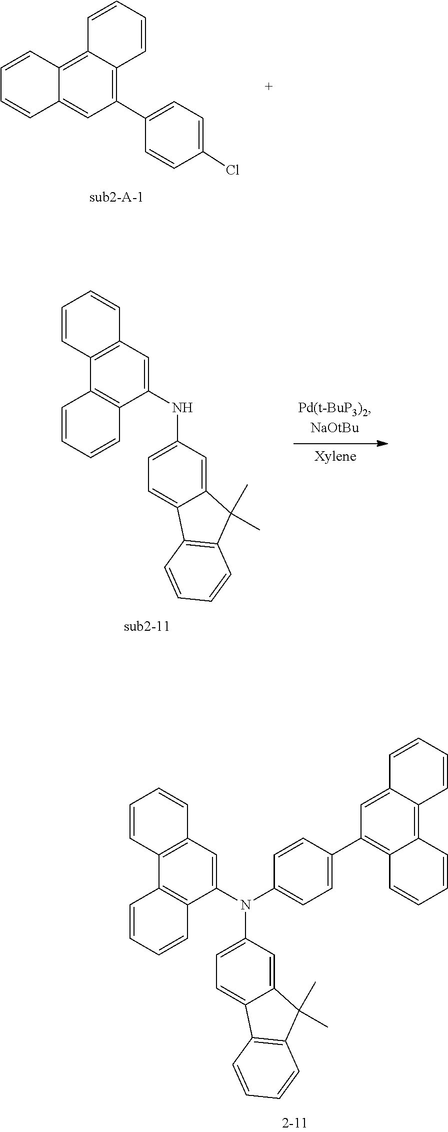 [SMILES: Clc1ccc(-c2cc3ccccc3c3ccccc23)cc1 ; CC1(C)c2ccccc2-c2ccc(Nc3cc4ccccc4c4ccccc34)cc21 ; CC1(C)c2ccccc2-c2ccc(N(c3ccc(-c4cc5ccccc5c5ccccc45)cc3)c3cc4ccccc4c4ccccc34)cc21]

Compound sub2-A-1 (10 g, 34.6 mmol), Compound sub2-11 (13.4 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.4 g of Compound 2-11. (Yield: 56%, MS: [M+H]⁺=638)

### Preparation Example 2-12: Preparation of Compound 2-12

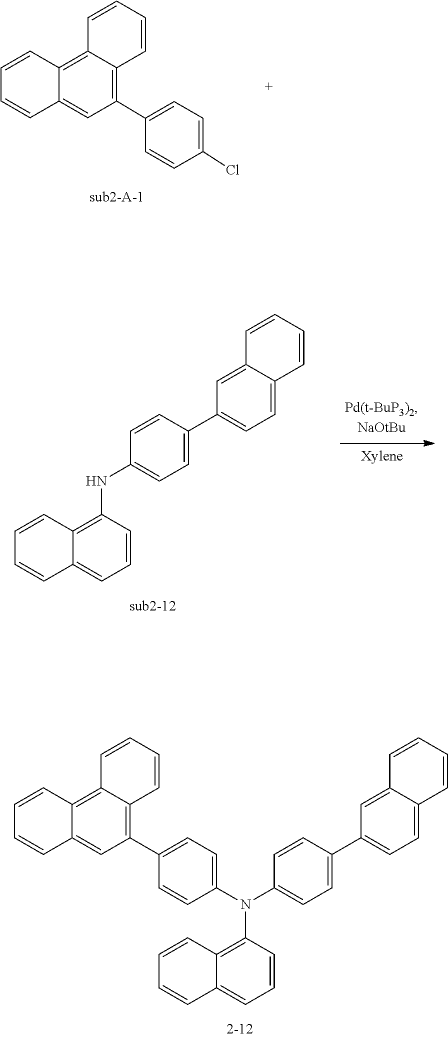 [SMILES: Clc1ccc(-c2cc3ccccc3c3ccccc23)cc1 ; c1ccc2cc(-c3ccc(Nc4cccc5ccccc45)cc3)ccc2c1 ; c1ccc2cc(-c3ccc(N(c4ccc(-c5cc6ccccc6c6ccccc56)cc4)c4cccc5ccccc45)cc3)ccc2c1]

Compound sub2-A-1 (10 g, 34.6 mmol), Compound sub2-12 (12 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11 g of Compound 2-12. (Yield: 53%, MS: [M+H]⁺=598)

### Preparation Example 2-13: Preparation of Compound 2-13

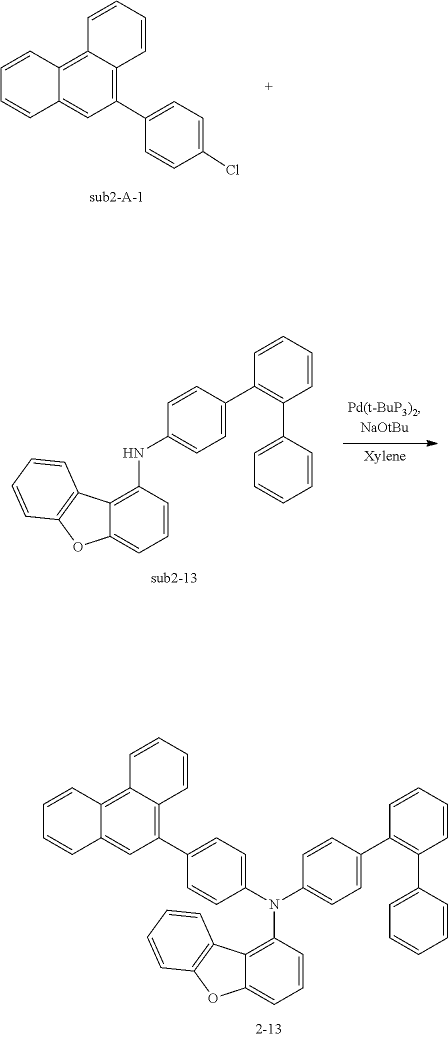 [SMILES: Clc1ccc(-c2cc3ccccc3c3ccccc23)cc1 ; c1ccc(-c2ccccc2-c2ccc(Nc3cccc4oc5ccccc5c34)cc2)cc1 ; c1ccc(-c2ccccc2-c2ccc(N(c3ccc(-c4cc5ccccc5c5ccccc45)cc3)c3cccc4oc5ccccc5c34)cc2)cc1]

Compound sub2-A-1 (10 g, 34.6 mmol), Compound sub2-13 (14.3 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 15.6 g of Compound 2-13. (Yield: 68%, MS: [M+H]⁺=664)

### Preparation Example 2-14: Preparation of Compound 2-14

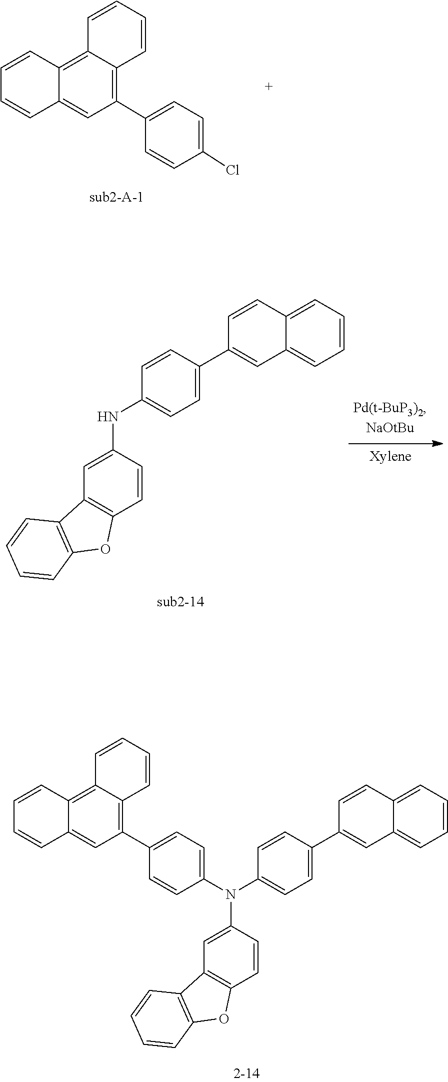 [SMILES: Clc1ccc(-c2cc3ccccc3c3ccccc23)cc1 ; c1ccc2cc(-c3ccc(Nc4ccc5oc6ccccc6c5c4)cc3)ccc2c1 ; c1ccc2cc(-c3ccc(N(c4ccc(-c5cc6ccccc6c6ccccc56)cc4)c4ccc5oc6ccccc6c5c4)cc3)ccc2c1]

Compound sub2-A-1 (10 g, 34.6 mmol), Compound sub2-14 (13.3 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 13.2 g of Compound 2-14. (Yield: 60%, MS: [M+H]⁺=638)

### Preparation Example 2-15: Preparation of Compound 2-15

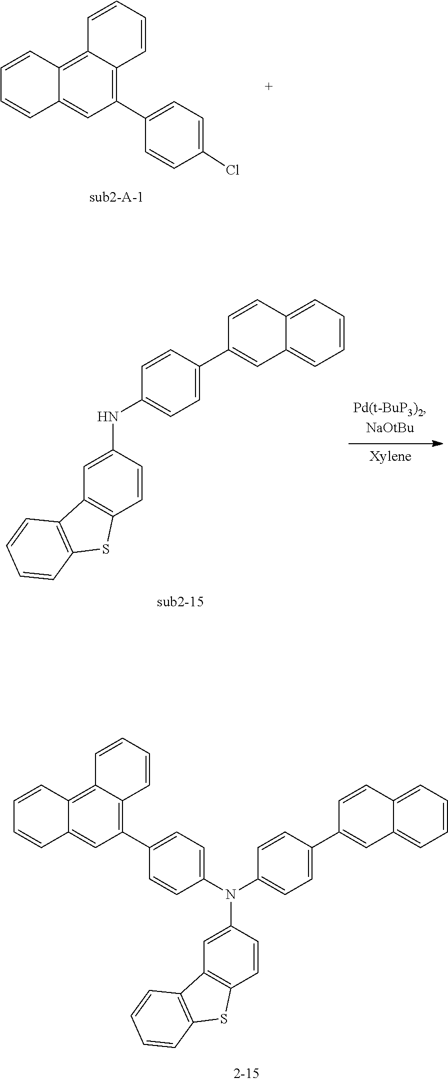 [SMILES: Clc1ccc(-c2cc3ccccc3c3ccccc23)cc1 ; c1ccc2cc(-c3ccc(Nc4ccc5sc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1]

Compound sub2-A-1 (10 g, 34.6 mmol), Compound sub2-15 (13.9 g, 34.6 mmol) and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12 g of Compound 2-15. (Yield: 53%, MS: [M+H]⁺=654)

### Preparation Example 2-16: Preparation of Compound 2-16

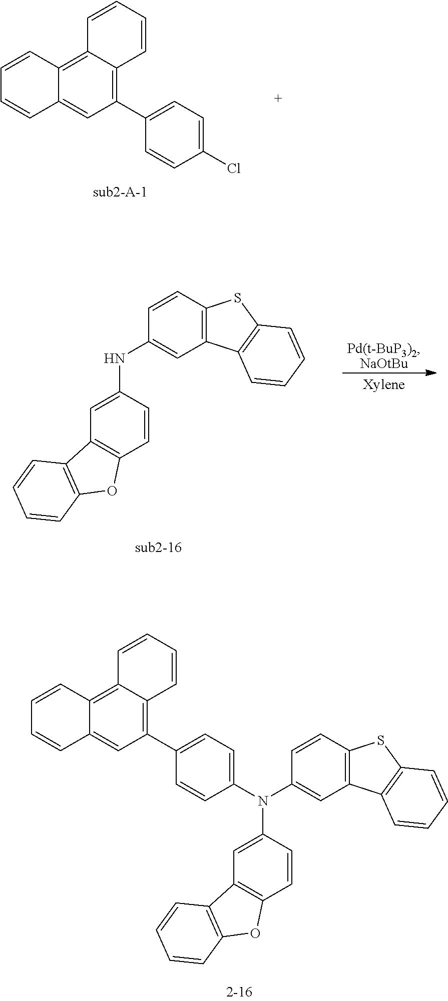

Compound sub2-A-1 (10 g, 34.6 mmol), Compound sub2-16 (12.7 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 13.7 g of Compound 2-16. (Yield: 64%, MS: [M+H]⁺=618)

### Preparation Example 2-17: Preparation of Compound 2-17

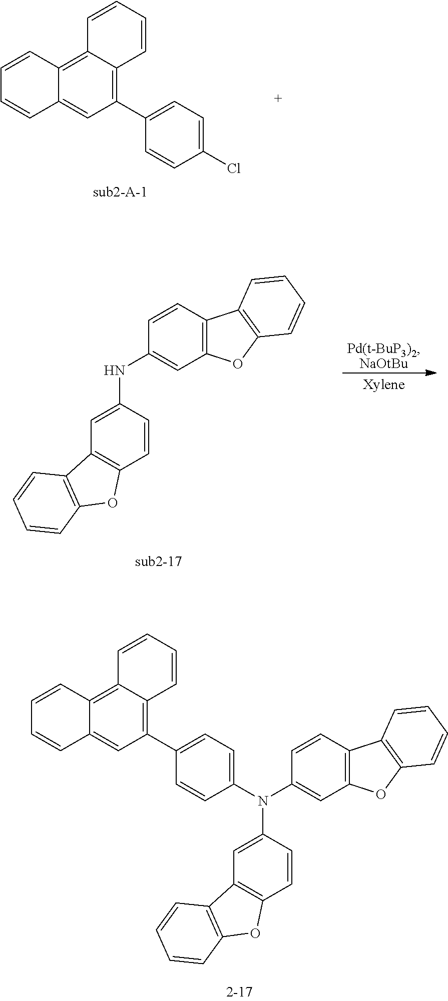

Compound sub2-A-1 (10 g, 34.6 mmol), Compound sub2-17 (12.1 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.5 g of Compound 2-17. (Yield: 55%, MS: [M+H]⁺=602)

### Preparation Example 2-18: Preparation of Compound 2-18

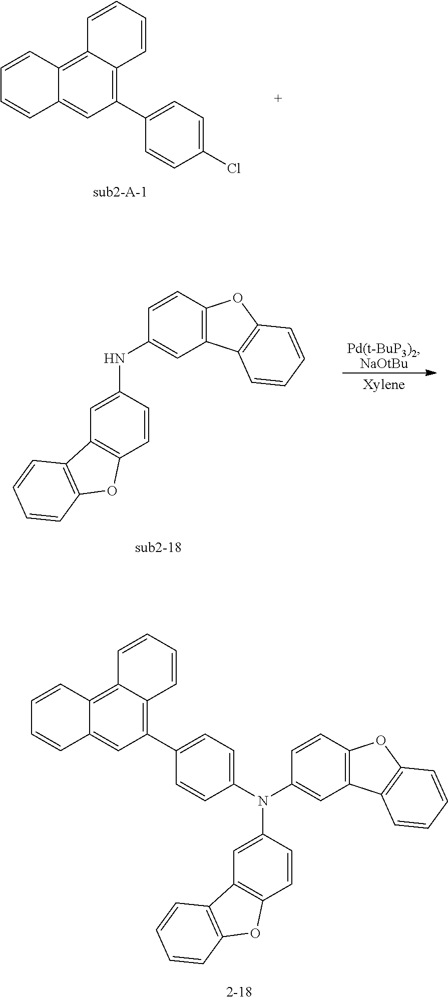

Compound sub2-A-1 (10 g, 34.6 mmol), Compound sub2-18 (12.1 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 14.4 g of Compound 2-18. (Yield: 69%, MS: [M+H]⁺=602)

### Preparation Example 2-19: Preparation of Compound 2-19

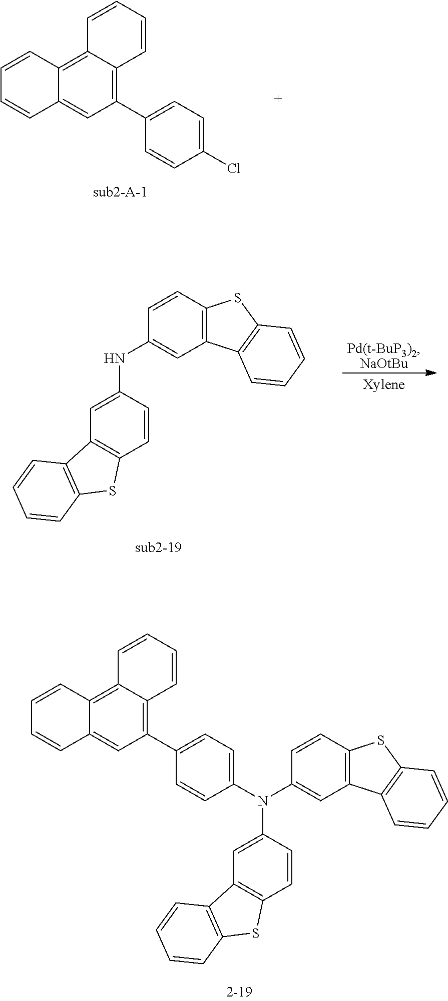

Compound sub2-A-1 (10 g, 34.6 mmol), Compound sub2-19 (13.2 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.4 g of Compound 2-19. (Yield: 52%, MS: [M+H]⁺=634)

### Preparation Example 2-20: Preparation of Compound 2-20

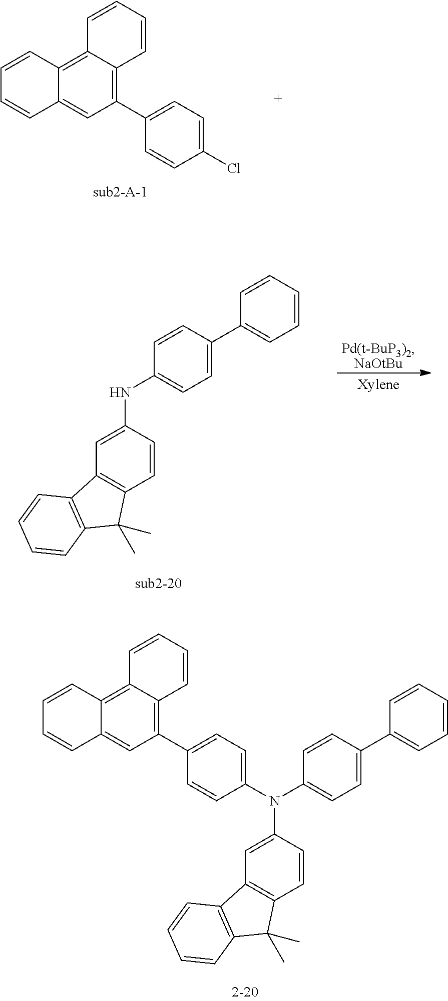

Compound sub2-A-1 (10 g, 34.6 mmol), Compound sub2-20 (12.5 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 13.2 g of Compound 2-20. (Yield: 62%, MS: [M+H]⁺=614)

### Preparation Example 2-21: Preparation of Compound 2-21

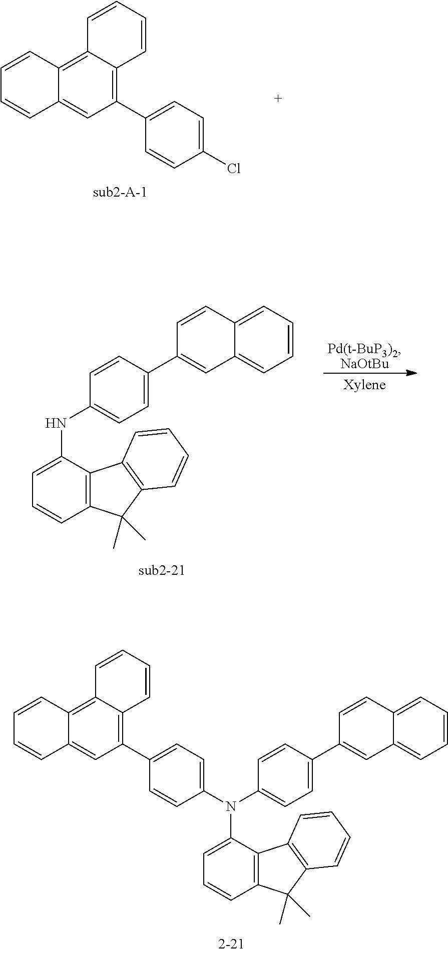

Compound sub2-A-1 (10 g, 34.6 mmol), Compound sub2-21 (14.3 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 14.2 g of Compound 2-21. (Yield: 62%, MS: [M+H]⁺=664)

### Preparation Example 2-22: Preparation of Compound 2-22

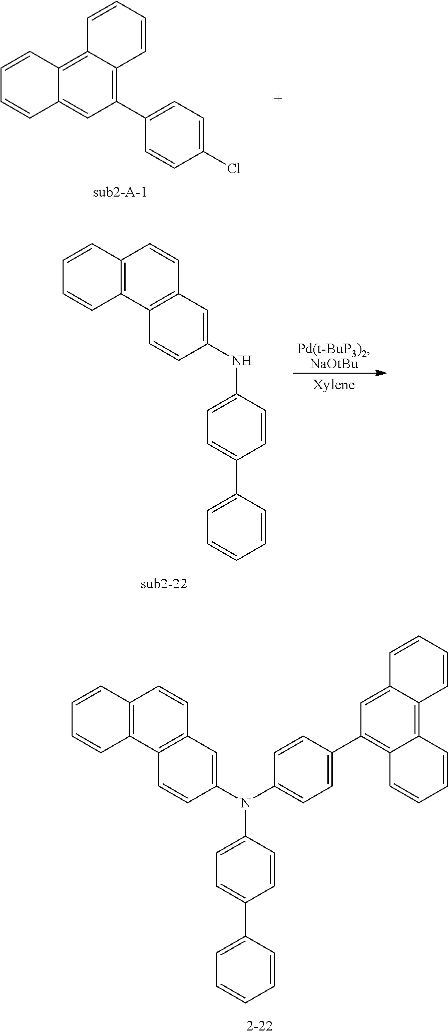

Compound sub2-A-1 (10 g, 34.6 mmol), Compound sub2-22 (12 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.2 g of Compound 2-22. (Yield: 54%, MS: [M+H]⁺=598)

### Preparation Example 2-23: Preparation of Compound 2-23

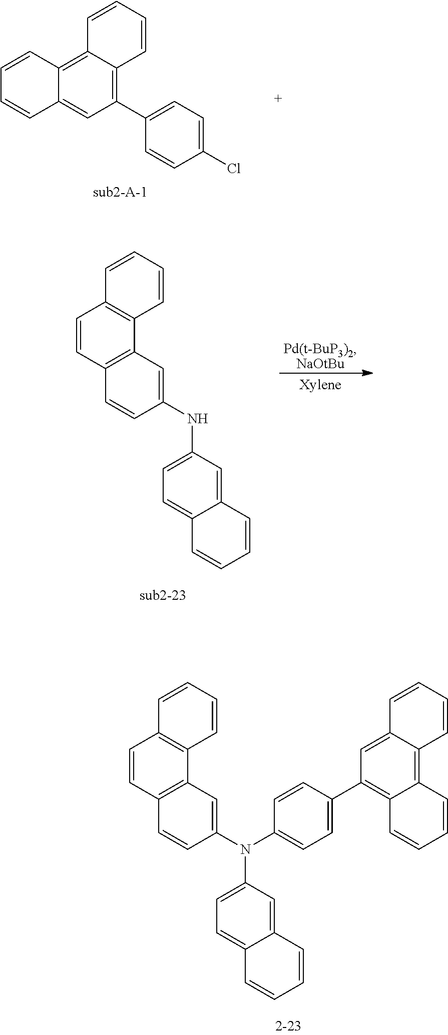

Compound sub2-A-1 (10 g, 34.6 mmol), Compound sub2-23 (11.1 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.9 g of Compound 2-23. (Yield: 60%, MS: [M+H]⁺=572)

### Preparation Example 2-24: Preparation of Compound 2-24

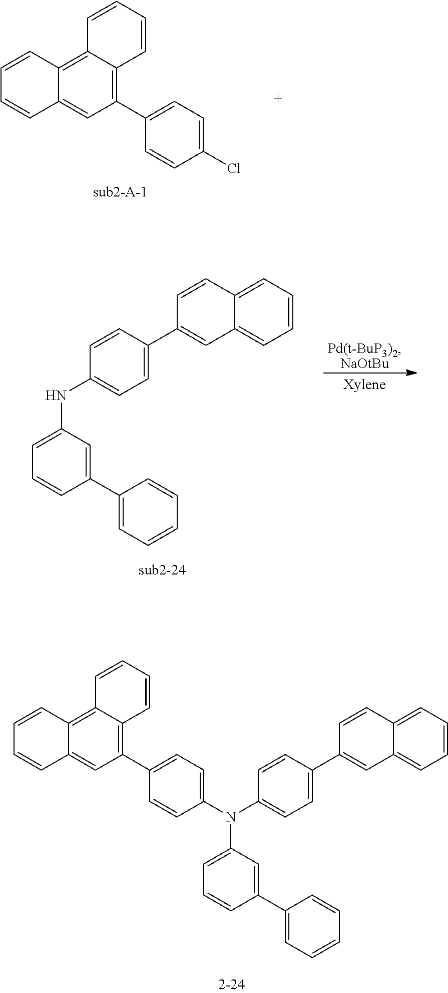

Compound sub2-A-1 (10 g, 34.6 mmol), Compound sub2-24 (12.9 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 13.6 g of Compound 2-24. (Yield: 63%, MS: [M+H]⁺=624)

### Preparation Example 2-25: Preparation of Compound 2-25

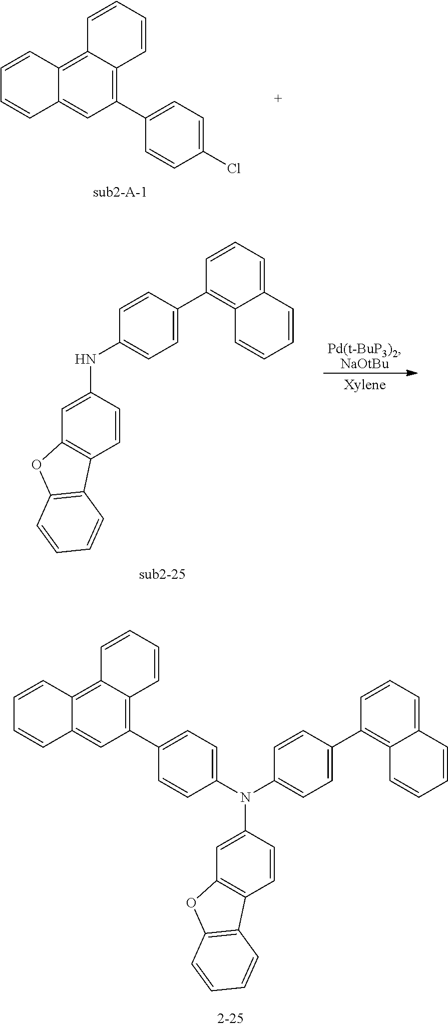

Compound sub2-A-1 (10 g, 34.6 mmol), Compound sub2-25 (13.3 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 14.3 g of Compound 2-25. (Yield: 65%, MS: [M+H]⁺=638)

### Preparation Example 2-26: Preparation of Compound 2-26

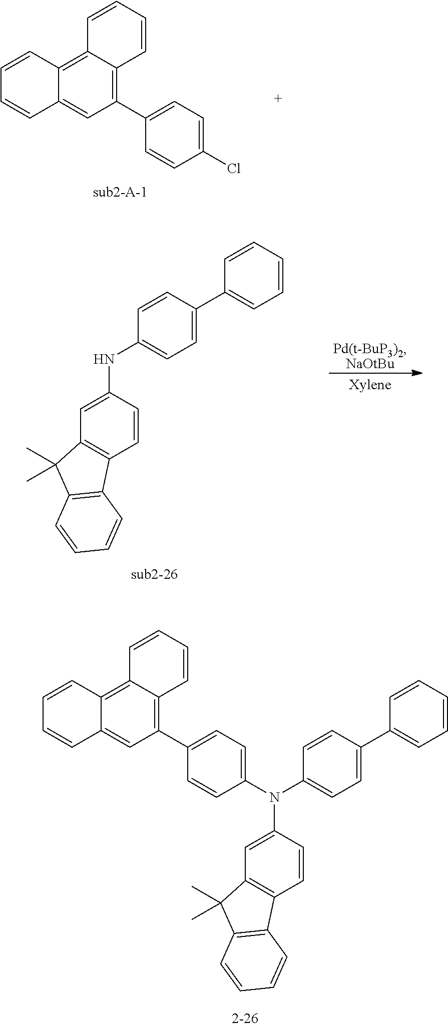

Compound sub2-A-1 (10 g, 34.6 mmol), Compound sub2-26 (12.5 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 10.8 g of Compound 2-26. (Yield: 51%, MS: [M+H]⁺=614)

### Preparation Example 2-27: Preparation of Compound 2-27

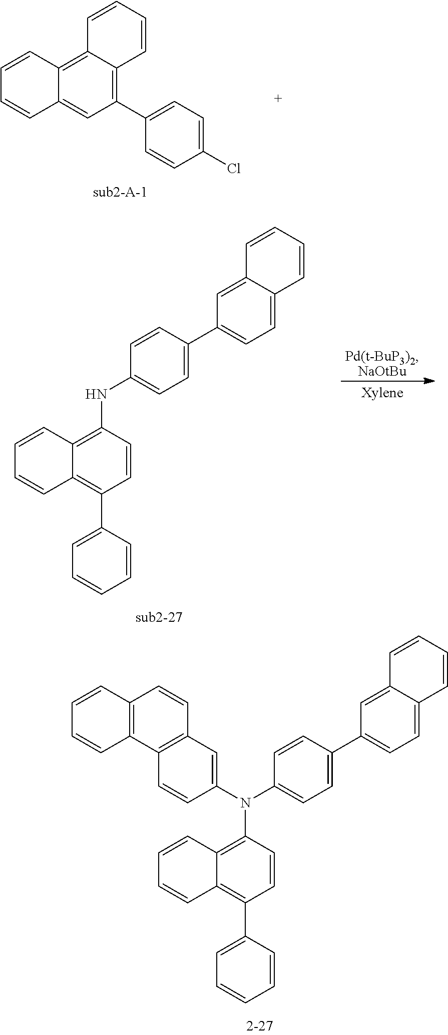

Compound sub2-A-1 (10 g, 34.6 mmol), Compound sub2-27 (14.6 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 16.1 g of Compound 2-27. (Yield: 69%, MS: [M+H]⁺=674)

### Preparation Example 2-28: Preparation of Compound 2-28

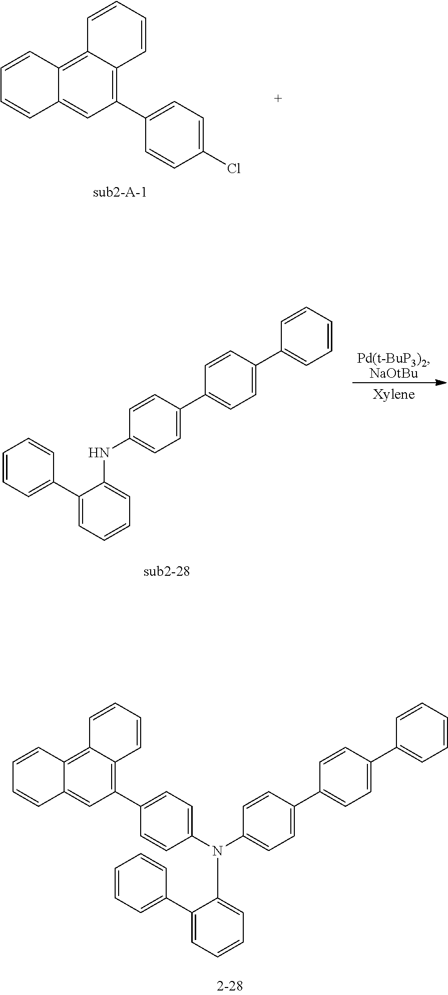

Compound sub2-A-1 (10 g, 34.6 mmol), Compound sub2-28 (13.8 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.2 g of Compound 2-28. (Yield: 50%, MS: [M+H]⁺=650)

### Preparation Example 2-29: Preparation of Compound 2-29

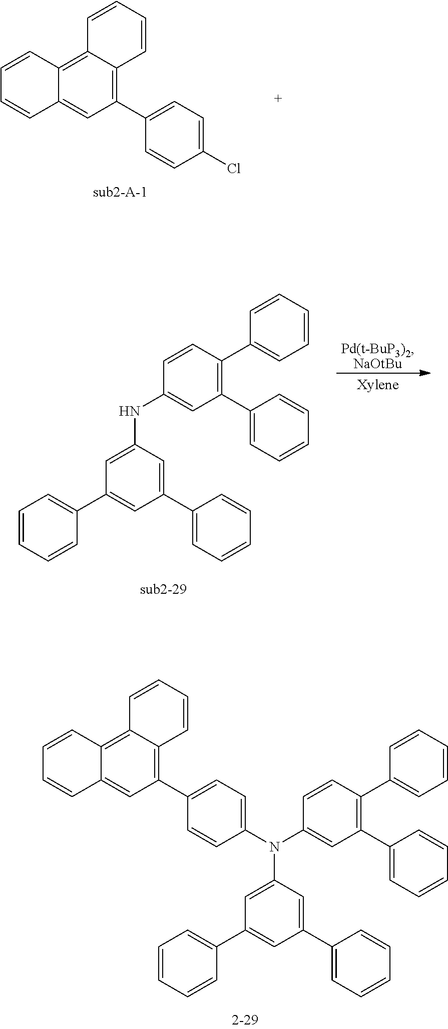

Compound sub2-A-1 (10 g, 34.6 mmol), Compound sub2-29 (16.4 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 17.1 g of Compound 2-29. (Yield: 68%, MS: [M+H]⁺=726)

### Preparation Example 2-30: Preparation of Compound 2-30

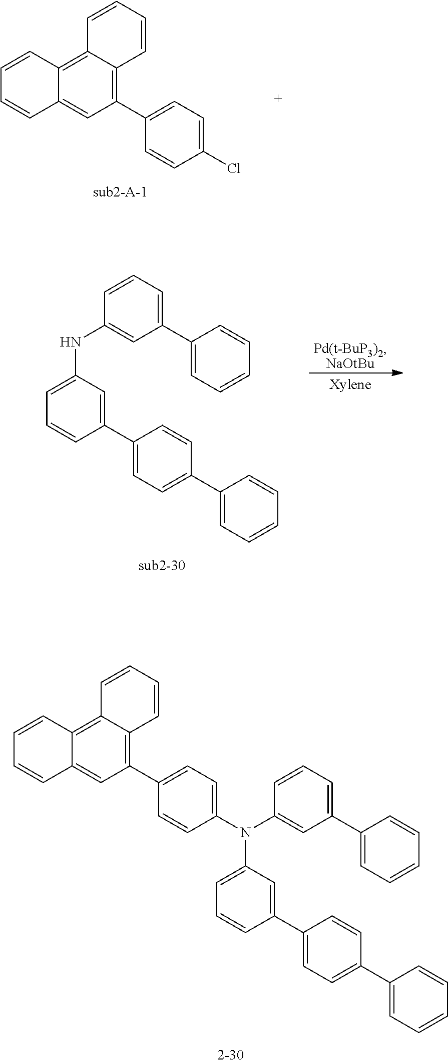

Compound sub2-A-1 (10 g, 34.6 mmol), Compound sub2-30 (13.8 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 14.4 g of Compound 2-30. (Yield: 64%, MS: [M+H]⁺=650)

### Preparation Example 2-31: Preparation of Compound 2-31

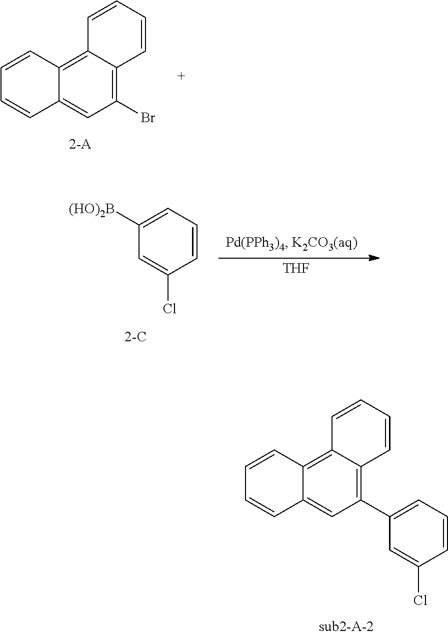 [SMILES: Brc1cc2ccccc2c2ccccc12 ; OB(O)c1cccc(Cl)c1 ; Clc1cccc(-c2cc3ccccc3c3ccccc23)c1 ; "THF"]

Compound 2-A (15 g, 58.3 mmol) and Compound 2-C (10 g, 64.2 mmol) were added to 300 ml of THF under a nitrogen atmosphere, and the mixture was stirred and refluxed. Then, potassium carbonate (16.1 g, 116.7 mmol) was dissolved in 48 ml of water, added thereto, and the mixture was sufficiently stirred and then tetrakis(triphenylphosphine)palladium(0) (1.3 g, 1.2 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 10.6 g of Compound sub2-A-2. (Yield: 63%, MS: [M+H]⁺=289)

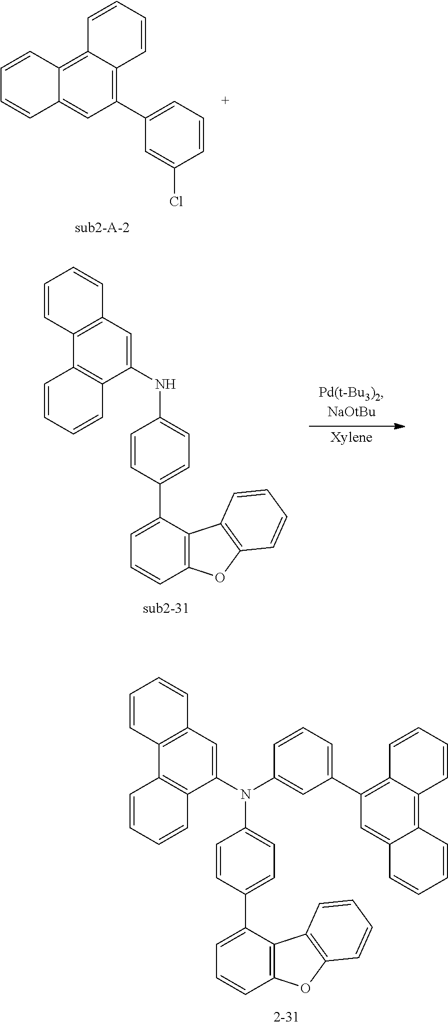 [SMILES: Clc1cccc(-c2cc3ccccc3c3ccccc23)c1 ; c1ccc2c(c1)cc(Nc1ccc(-c3cccc4oc5ccccc5c34)cc1)c1ccccc12 ; c1cc(-c2cc3ccccc3c3ccccc23)cc(N(c2ccc(-c3cccc4oc5ccccc5c34)cc2)c2cc3ccccc3c3ccccc23)c1]

Compound sub2-A-2 (10 g, 34.6 mmol), Compound sub2-31 (15.1 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 16.7 g of Compound 2-31. (Yield: 70%, MS: [M+H]⁺=688)

### Preparation Example 2-32: Preparation of Compound 2-32

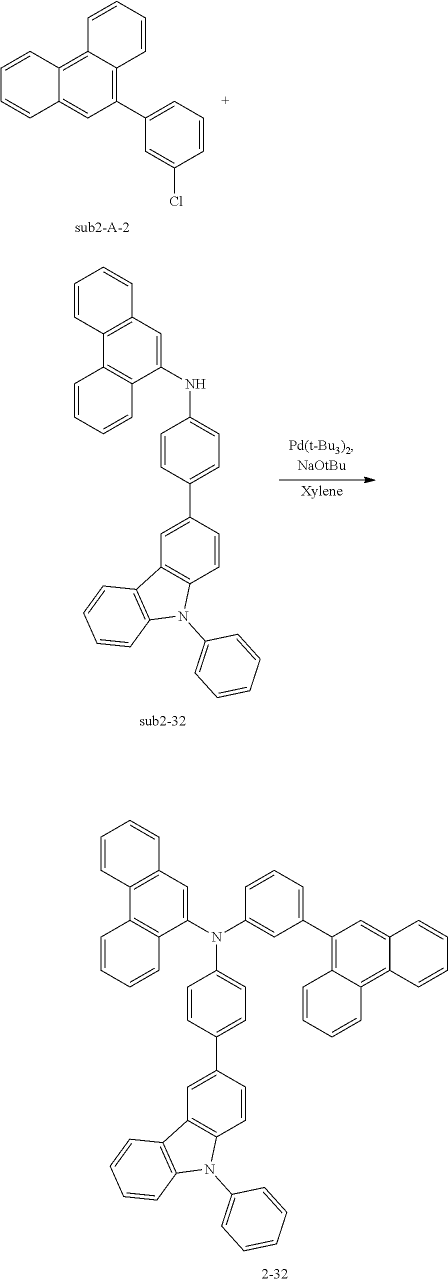 [SMILES: Clc1cccc(-c2cc3ccccc3c3ccccc23)c1 ; c1ccc(-n2c3ccccc3c3cc(-c4ccc(Nc5cc6ccccc6c6ccccc56)cc4)ccc32)cc1 ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1]

Compound sub2-A-2 (10 g, 34.6 mmol), Compound sub2-32 (17.7 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 16.6 g of Compound 2-32. (Yield: 63%, MS: [M+H]⁺=763)

### Preparation Example 2-33: Preparation of Compound 2-33

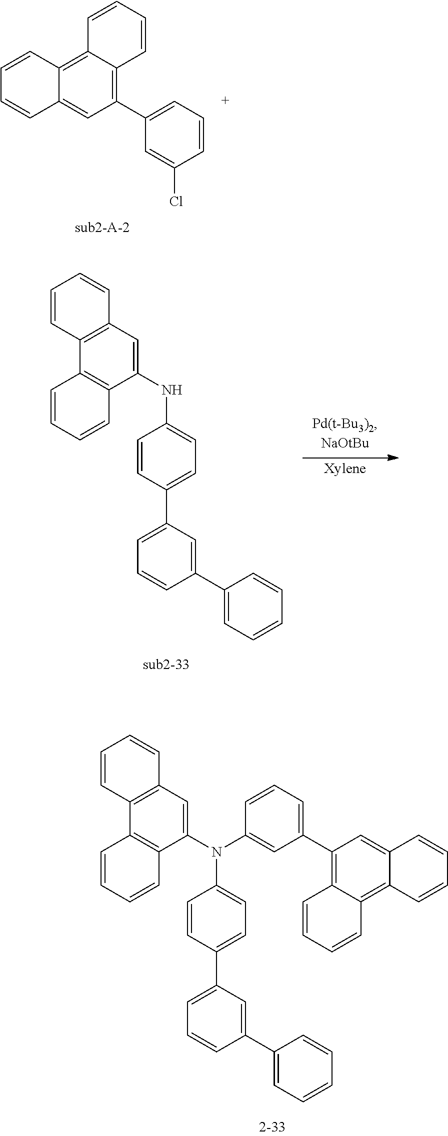 [SMILES: Clc1cccc(-c2cc3ccccc3c3ccccc23)c1 ; c1ccc(-c2cccc(-c3ccc(Nc4cc5ccccc5c5ccccc45)cc3)c2)cc1 ; c1ccc(-c2cccc(-c3ccc(N(c4cccc(-c5cc6ccccc6c6ccccc56)c4)c4cc5ccccc5c5ccccc45)cc3)c2)cc1]

Compound sub2-A-2 (10 g, 34.6 mmol), Compound sub2-33 (14.6 g, 34.6 mmol) and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.6 g of Compound 2-33. (Yield: 54%, MS: [M+H]⁺=674)

### Preparation Example 2-34: Preparation of Compound 2-34

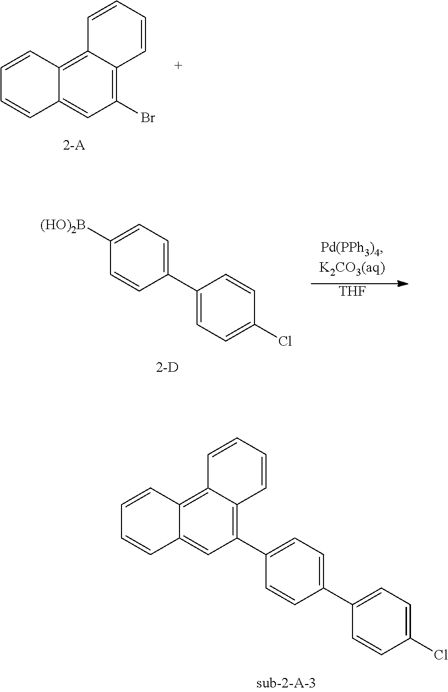 [SMILES: Brc1cc2ccccc2c2ccccc12 ; OB(O)c1ccc(-c2ccc(Cl)cc2)cc1 ; Clc1ccc(-c2ccc(-c3cc4ccccc4c4ccccc34)cc2)cc1 ; "THF"]

Compound 2-A (15 g, 58.3 mmol) and Compound 2-D (14.9 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water, added thereto, and the mixture was sufficiently stirred and then tetrakis(triphenylphosphine)palladium(0) (1.3 g, 1.2 mmol) was added. After 10-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 16.8 g of Compound sub2-A-3. (Yield: 79%, MS: [M+H]⁺=365)

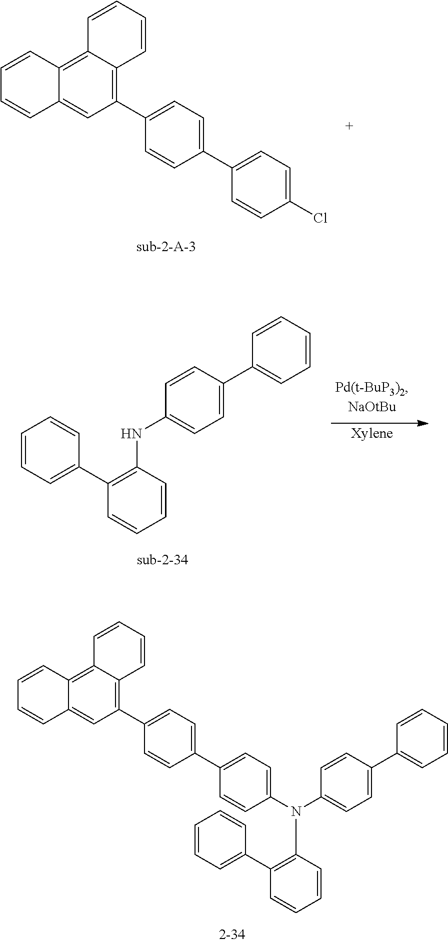 [SMILES: Clc1ccc(-c2ccc(-c3cc4ccccc4c4ccccc34)cc2)cc1 ; c1ccc(-c2ccc(Nc3ccccc3-c3ccccc3)cc2)cc1 ; "Xylene" ; c1ccc(-c2ccc(N(c3ccc(-c4ccc(-c5cc6ccccc6c6ccccc56)cc4)cc3)c3ccccc3-c3ccccc3)cc2)cc1]

Compound sub2-A-3 (10 g, 27.4 mmol), Compound sub2-34 (8.8 g, 27.4 mmol) and sodium tert-butoxide (3.4 g, 35.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.2 g of Compound 2-34. (Yield: 63%, MS: [M+H]⁺=650)

### Preparation Example 2-35: Preparation of Compound 2-35

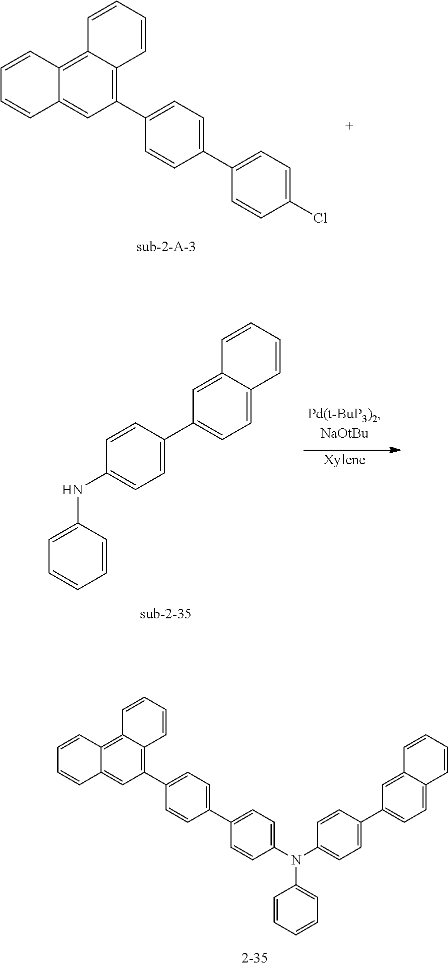 [SMILES: Clc1ccc(-c2ccc(-c3cc4ccccc4c4ccccc34)cc2)cc1 ; c1ccc(Nc2ccc(-c3ccc4ccccc4c3)cc2)cc1 ; "Xylene" ; c1ccc(N(c2ccc(-c3ccc(-c4cc5ccccc5c5ccccc45)cc3)cc2)c2ccc(-c3ccc4ccccc4c3)cc2)cc1]

Compound sub2-A-3 (10 g, 27.4 mmol), Compound sub2-35 (8.1 g, 27.4 mmol) and sodium tert-butoxide (3.4 g, 35.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 8.7 g of Compound 2-35. (Yield: 51%, MS: [M+H]⁺=624)

### Preparation Example 2-36: Preparation of Compound 2-36

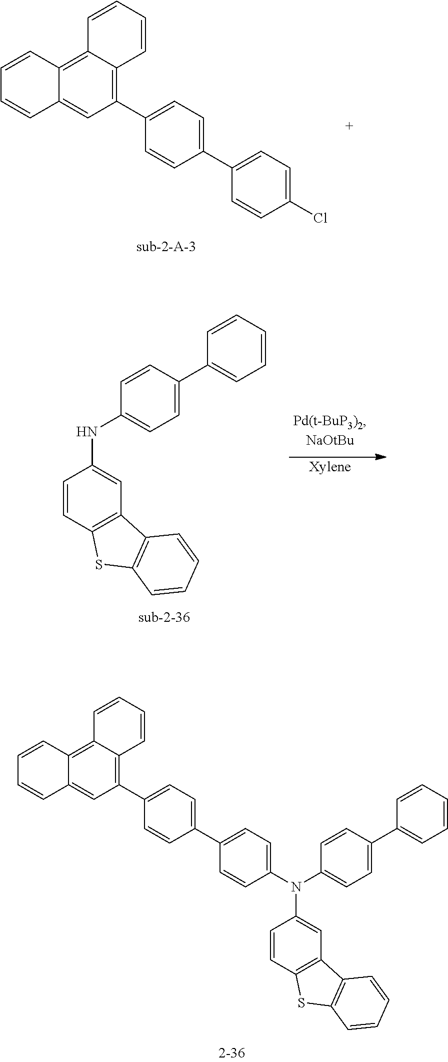 [SMILES: Clc1ccc(-c2ccc(-c3cc4ccccc4c4ccccc34)cc2)cc1 ; c1ccc(-c2ccc(Nc3ccc4sc5ccccc5c4c3)cc2)cc1 ; "Xylene" ; c1ccc(-c2ccc(N(c3ccc(-c4ccc(-c5cc6ccccc6c6ccccc56)cc4)cc3)c3ccc4sc5ccccc5c4c3)cc2)cc1]

Compound sub2-A-3 (10 g, 27.4 mmol), Compound sub2-36 (9.6 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.1 g of Compound 2-36. (Yield: 65%, MS: [M+H]⁺=680)

### Preparation Example 2-37: Preparation of Compound 2-37

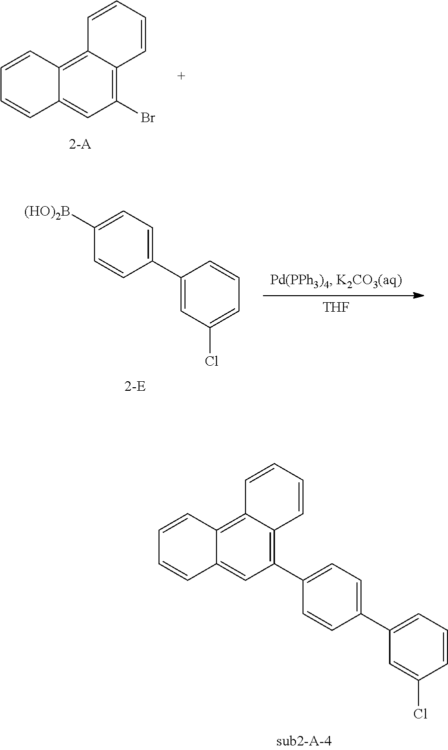 [SMILES: Brc1cc2ccccc2c2ccccc12 ; OB(O)c1ccc(-c2cccc(Cl)c2)cc1 ; Clc1cccc(-c2ccc(-c3cc4ccccc4c4ccccc34)cc2)c1 ; "THF"]

Compound 2-A (15 g, 58.3 mmol) and Compound 2-E (14.9 g, 64.2 mmol) were added to 300 ml of THF under a nitrogen atmosphere, and the mixture was stirred and refluxed. Then, potassium carbonate (16.1 g, 116.7 mmol) was dissolved in 48 ml of water, added thereto, and the mixture was sufficiently stirred and then tetrakis(triphenylphosphine)palladium(0) (1.3 g, 1.2 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.2 g of Compound sub2-A-4. (Yield: 67%, MS: [M+H]⁺=365)

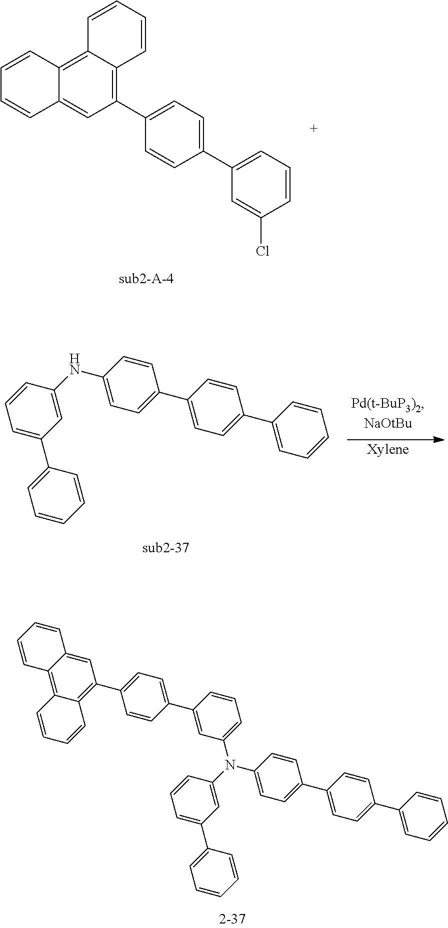 [SMILES: Clc1cccc(-c2ccc(-c3cc4ccccc4c4ccccc34)cc2)c1 ; c1ccc(-c2ccc(-c3ccc(Nc4cccc(-c5ccccc5)c4)cc3)cc2)cc1 ; "Xylene" ; c1ccc(-c2ccc(-c3ccc(N(c4cccc(-c5ccccc5)c4)c4cccc(-c5ccc(-c6cc7ccccc7c7ccccc67)cc5)c4)cc3)cc2)cc1]

Compound sub2-A-4 (10 g, 27.4 mmol), Compound sub2-37 (10.9 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 13.9 g of Compound 2-37. (Yield: 70%, MS: [M+H]⁺=726)

### Preparation Example 2-38: Preparation of Compound 2-38

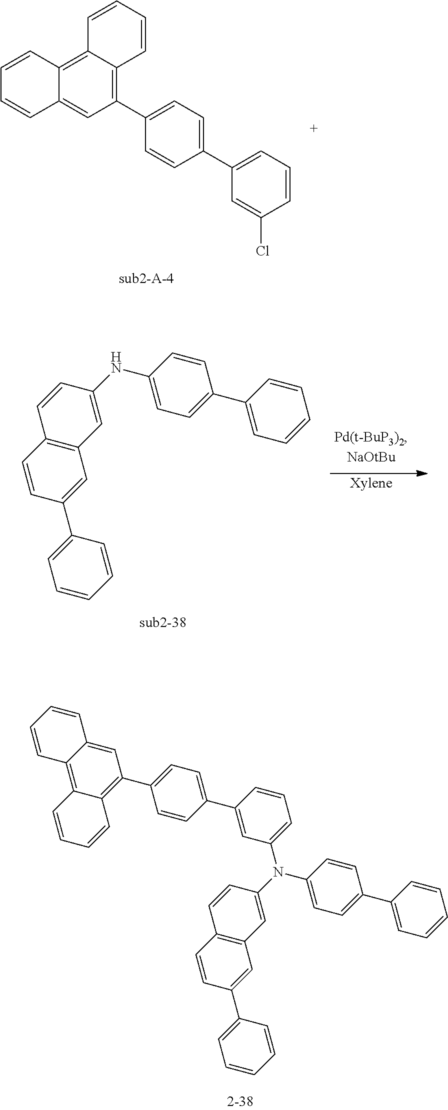 [SMILES: Clc1cccc(-c2ccc(-c3cc4ccccc4c4ccccc34)cc2)c1 ; c1ccc(-c2ccc(Nc3ccc4ccc(-c5ccccc5)cc4c3)cc2)cc1 ; "Xylene" ; c1ccc(-c2ccc(N(c3cccc(-c4ccc(-c5cc6ccccc6c6ccccc56)cc4)c3)c3ccc4ccc(-c5ccccc5)cc4c3)cc2)cc1]

Compound sub2-A-4 (10 g, 27.4 mmol), Compound sub2-38 (10.2 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 10.7 g of Compound 2-38. (Yield: 56%, MS: [M+H]⁺=700)

### Preparation Example 2-39: Preparation of Compound 2-39

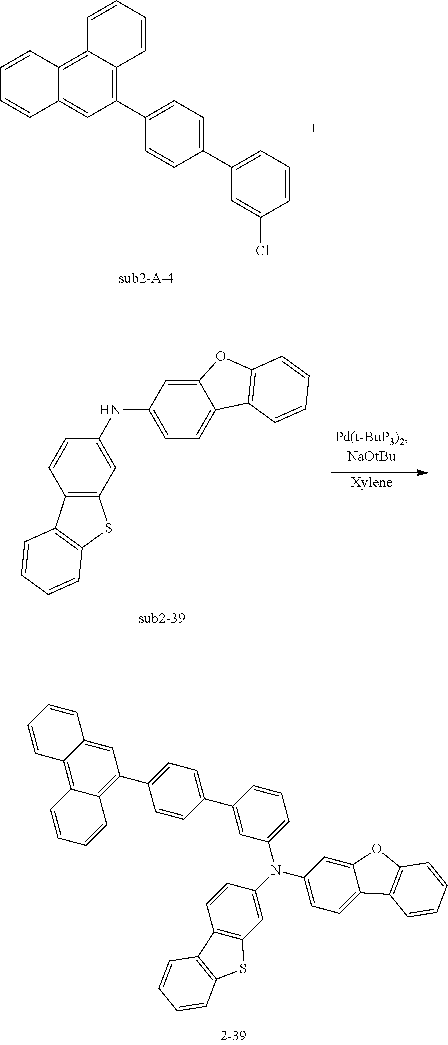 [SMILES: Clc1cccc(-c2ccc(-c3cc4ccccc4c4ccccc34)cc2)c1 ; c1ccc2c(c1)oc1cc(Nc3ccc4c(c3)sc3ccccc34)ccc12 ; "Xylene" ; c1cc(-c2ccc(-c3cc4ccccc4c4ccccc34)cc2)cc(N(c2ccc3c(c2)oc2ccccc23)c2ccc3c(c2)sc2ccccc23)c1]

Compound sub2-A-4 (10 g, 27.4 mmol), Compound sub2-39 (10 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.8 g of Compound 2-39. (Yield: 62%, MS: [M+H]⁺=694)

### Preparation Example 2-40: Preparation of Compound 2-40

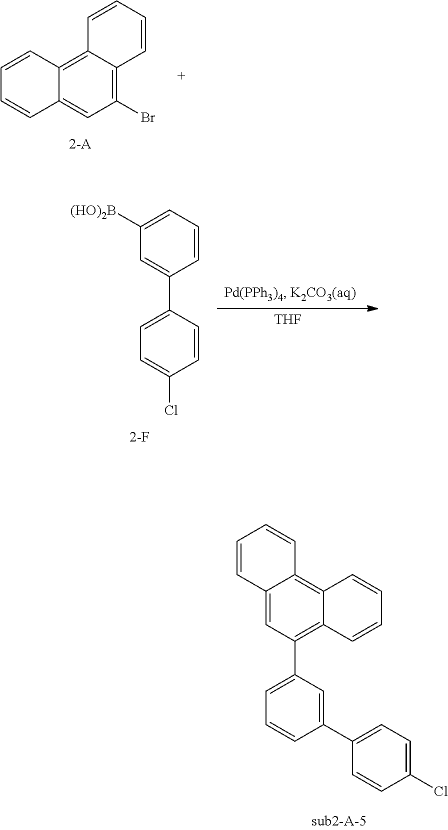 [SMILES: Brc1cc2ccccc2c2ccccc12 ; OB(O)c1cccc(-c2ccc(Cl)cc2)c1 ; Clc1ccc(-c2cccc(-c3cc4ccccc4c4ccccc34)c2)cc1 ; "THF"]

Compound 2-A (15 g, 58.3 mmol) and Compound 2-F (14.9 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water, added thereto, and the mixture was sufficiently stirred and then tetrakis(triphenylphosphine)palladium(0) (1.3 g, 1.2 mmol) was added. After 8-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.4 g of Compound sub2-A-5. (Yield: 68%, MS: [M+H]⁺=365)

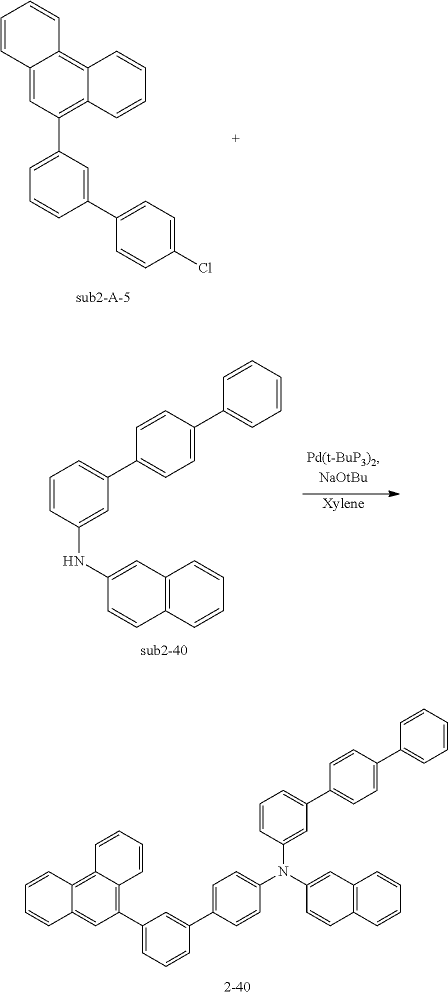 [SMILES: Clc1ccc(-c2cccc(-c3cc4ccccc4c4ccccc34)c2)cc1 ; c1ccc(-c2ccc(-c3cccc(Nc4ccc5ccccc5c4)c3)cc2)cc1 ; "Xylene" ; c1ccc(-c2ccc(-c3cccc(N(c4ccc(-c5cccc(-c6cc7ccccc7c7ccccc67)c5)cc4)c4ccc5ccccc5c4)c3)cc2)cc1]

Compound sub2-A-5 (10 g, 27.4 mmol), Compound sub2-40 (10.2 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 10.7 g of Compound 2-40. (Yield: 56%, MS: [M+H]⁺=700)

### Preparation Example 2-41: Preparation of Compound 2-41

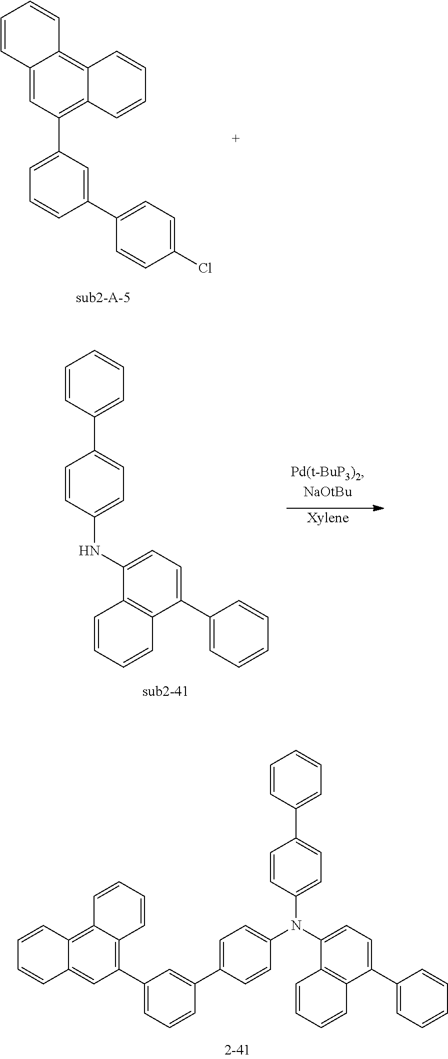 [SMILES: Clc1ccc(-c2cccc(-c3cc4ccccc4c4ccccc34)c2)cc1 ; c1ccc(-c2ccc(Nc3ccc(-c4ccccc4)c4ccccc34)cc2)cc1 ; "Xylene" ; c1ccc(-c2ccc(N(c3ccc(-c4cccc(-c5cc6ccccc6c6ccccc56)c4)cc3)c3ccc(-c4ccccc4)c4ccccc34)cc2)cc1]

Compound sub2-A-5 (10 g, 27.4 mmol), Compound sub2-41 (10.2 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 9.8 g of Compound 2-41. (Yield: 51%, MS: [M+H]⁺=700)

### Preparation Example 2-42: Preparation of Compound 2-42

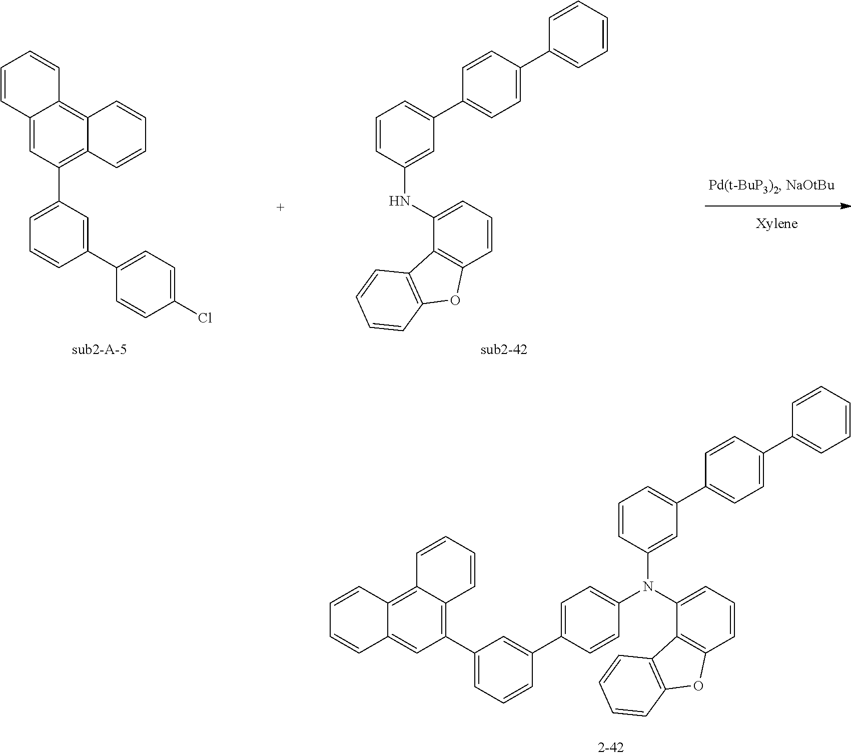 [SMILES: Clc1ccc(-c2cccc(-c3cc4ccccc4c4ccccc34)c2)cc1 ; c1ccc(-c2ccc(-c3cccc(Nc4cccc5oc6ccccc6c45)c3)cc2)cc1 ; c1ccc(-c2ccc(-c3cccc(N(c4ccc(-c5cccc(-c6cc7ccccc7c7ccccc67)c5)cc4)c4cccc5oc6ccccc6c45)c3)cc2)cc1]

Compound sub2-A-5 (10 g, 27.4 mmol), Compound sub2-42 (11.3 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.5 g of Compound 2-42. (Yield: 57%, MS: [M+H]⁺=740)

### Preparation Example 2-43: Preparation of Compound 2-43

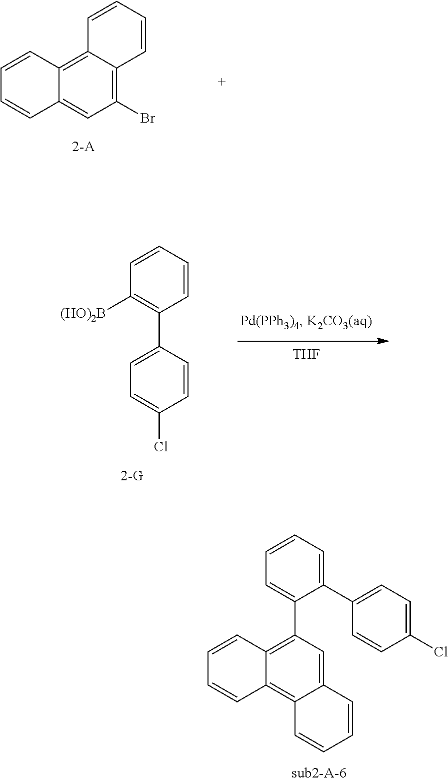 [SMILES: Brc1cc2ccccc2c2ccccc12 ; OB(O)c1ccccc1-c1ccc(Cl)cc1 ; Clc1ccc(-c2ccccc2-c2cc3ccccc3c3ccccc23)cc1 ; "THF"]

Compound 2-A (15 g, 58.3 mmol) and Compound 2-G (14.9 g, 64.2 mmol) were added to 300 ml of THF under a nitrogen atmosphere, and the mixture was stirred and refluxed. Then, potassium carbonate (16.1 g, 116.7 mmol) was dissolved in 48 ml of water, added thereto, and the mixture was sufficiently stirred and then tetrakis(triphenylphosphine)palladium(0) (1.3 g, 1.2 mmol) was added. After 9-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 14.7 g of Compound sub2-A-6. (Yield: 69%, MS: [M+H]⁺=365)

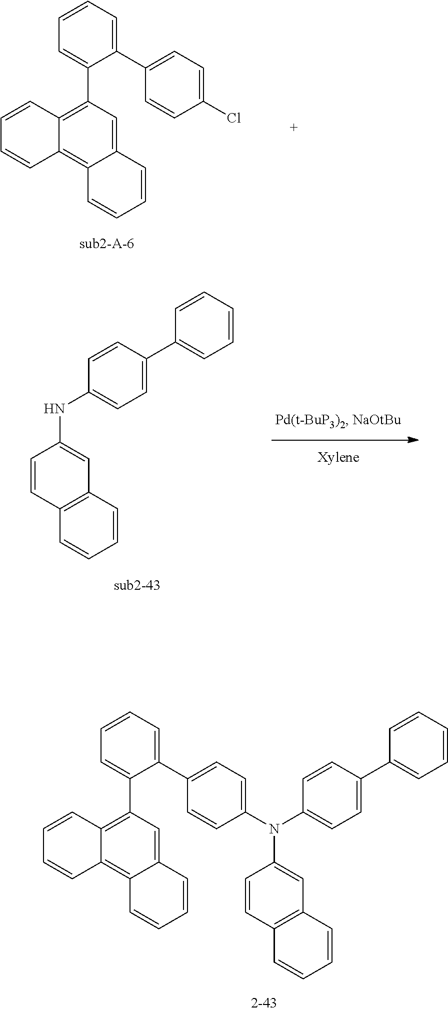 [SMILES: Clc1ccc(-c2ccccc2-c2cc3ccccc3c3ccccc23)cc1 ; c1ccc(-c2ccc(Nc3ccc4ccccc4c3)cc2)cc1 ; c1ccc(-c2ccc(N(c3ccc(-c4ccccc4-c4cc5ccccc5c5ccccc45)cc3)c3ccc4ccccc4c3)cc2)cc1]

Compound sub2-A-6 (10 g, 27.4 mmol), Compound sub2-43 (8.1 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 9.7 g of Compound 2-43. (Yield: 57%, MS: [M+H]⁺=624)

### Preparation Example 2-44: Preparation of Compound 2-44

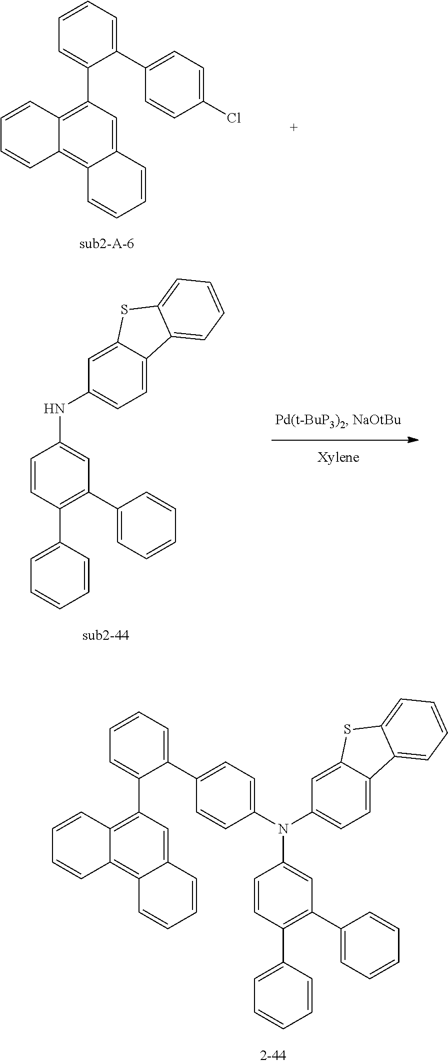 [SMILES: Clc1ccc(-c2ccccc2-c2cc3ccccc3c3ccccc23)cc1 ; c1ccc(-c2ccc(Nc3ccc4c(c3)sc3ccccc34)cc2-c2ccccc2)cc1 ; c1ccc(-c2ccc(N(c3ccc(-c4ccccc4-c4cc5ccccc5c5ccccc45)cc3)c3ccc4c(c3)sc3ccccc34)cc2-c2ccccc2)cc1]

Compound sub2-A-6 (10 g, 27.4 mmol), Compound sub2-44 (11.7 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12 g of Compound 2-44. (Yield: 58%, MS: [M+H]⁺=756)

### Preparation Example 2-45: Preparation of Compound 2-45

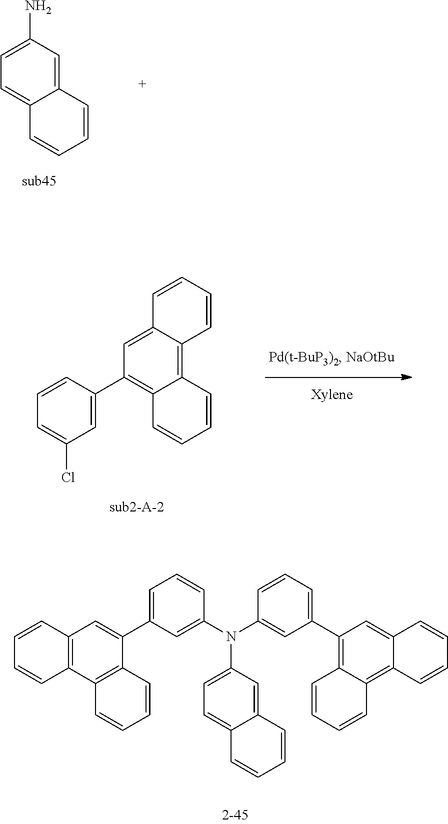 [SMILES: Nc1ccc2ccccc2c1 ; Clc1cccc(-c2cc3ccccc3c3ccccc23)c1 ; c1cc(-c2cc3ccccc3c3ccccc23)cc(N(c2cccc(-c3cc4ccccc4c4ccccc34)c2)c2ccc3ccccc3c2)c1]

Compound sub45 (10 g, 70.3 mmol), Compound sub2-A-2 (42.6 g, 147.7 mmol) and sodium tert-butoxide (16.9 g, 175.8 mmol) were added to 200 ml of xylene under a nitrogen atmosphere, and the mixture was stirred and refluxed. Then, bis(tri-tert-butylphosphine)palladium(0) (0.7 g, 1.4 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 31 g of Compound 2-45. (Yield: 68%, MS: [M+H]⁺=648)

### Preparation Example 2-46: Preparation of Compound 2-46

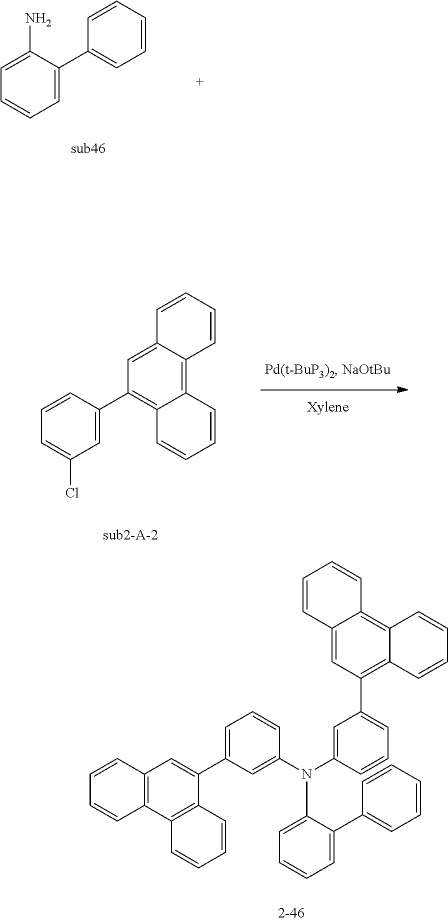 [SMILES: Nc1ccccc1-c1ccccc1 ; Clc1cccc(-c2cc3ccccc3c3ccccc23)c1 ; c1ccc(-c2ccccc2N(c2cccc(-c3cc4ccccc4c4ccccc34)c2)c2cccc(-c3cc4ccccc4c4ccccc34)c2)cc1]

Compound sub46 (10 g, 59.1 mmol), Compound sub2-A-2 (35.8 g, 124.1 mmol) and sodium tert-butoxide (14.2 g, 147.7 mmol) were added to 200 ml of xylene under a nitrogen atmosphere, and the mixture was stirred and refluxed. Then, bis(tri-tert-butylphosphine)palladium(0) (0.6 g, 1.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 26.7 g of Compound 2-46. (Yield: 67%, MS: [M+H]⁺=674)

### Preparation Example 2-47: Preparation of Compound 2-47

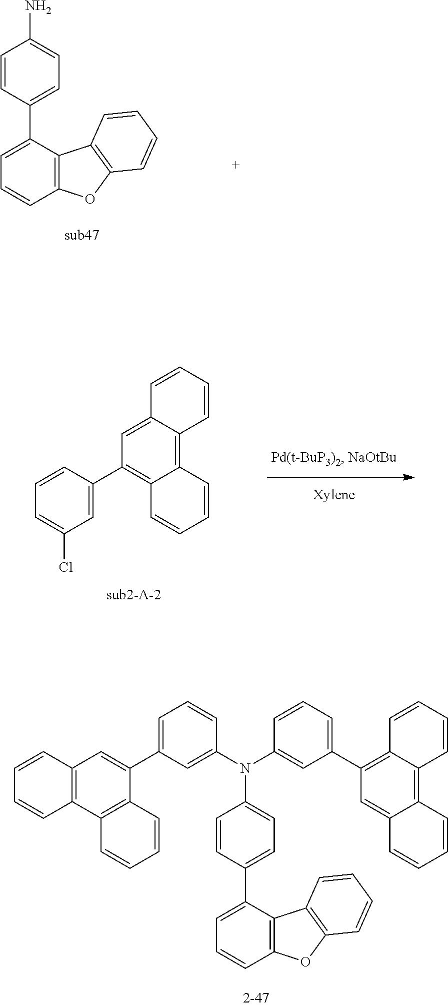 [SMILES: Nc1ccc(-c2cccc3oc4ccccc4c23)cc1 ; Clc1cccc(-c2cc3ccccc3c3ccccc23)c1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1]

Compound sub47 (10 g, 38.6 mmol), Compound sub2-A-2 (23.4 g, 81 mmol) and sodium tert-butoxide (9.3 g, 96.4 mmol) were added to 200 ml of xylene under a nitrogen atmosphere, and the mixture was stirred and refluxed. Then, bis(tri-tert-butylphosphine)palladium(0) (0.4 g, 0.8 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 15 g of Compound 2-47. (Yield: 51%, MS: [M+H]⁺=764)

### Preparation Example 2-48: Preparation of Compound 2-48

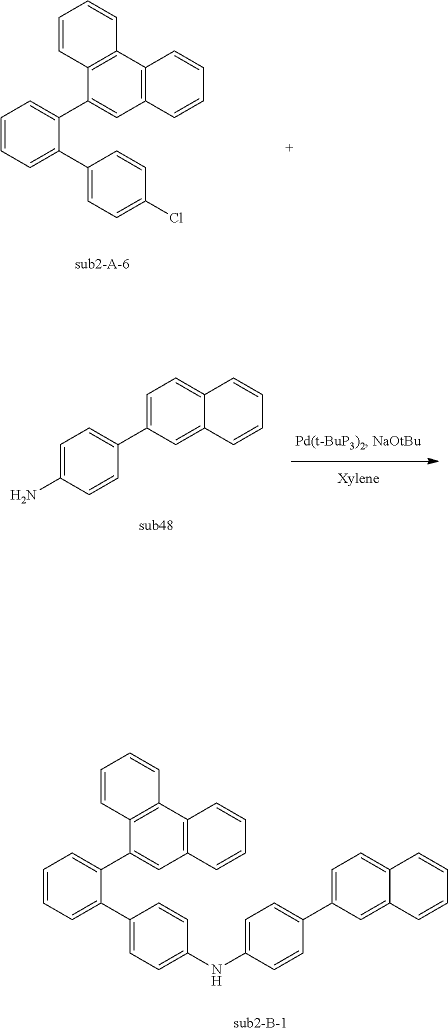 [SMILES: Clc1ccc(-c2ccccc2-c2cc3ccccc3c3ccccc23)cc1 ; Nc1ccc(-c2ccc3ccccc3c2)cc1 ; c1ccc(-c2cc3ccccc3c3ccccc23)c(-c2ccc(Nc3ccc(-c4ccc5ccccc5c4)cc3)cc2)c1]

Compound sub1-A-6 (10 g, 27.4 mmol), Compound sub48 (6 g, 27.4 mmol) and sodium tert-butoxide (2.9 g, 30.1 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 9 g of Compound sub2-B-1. (Yield: 60%, MS: [M+H]⁺=548)

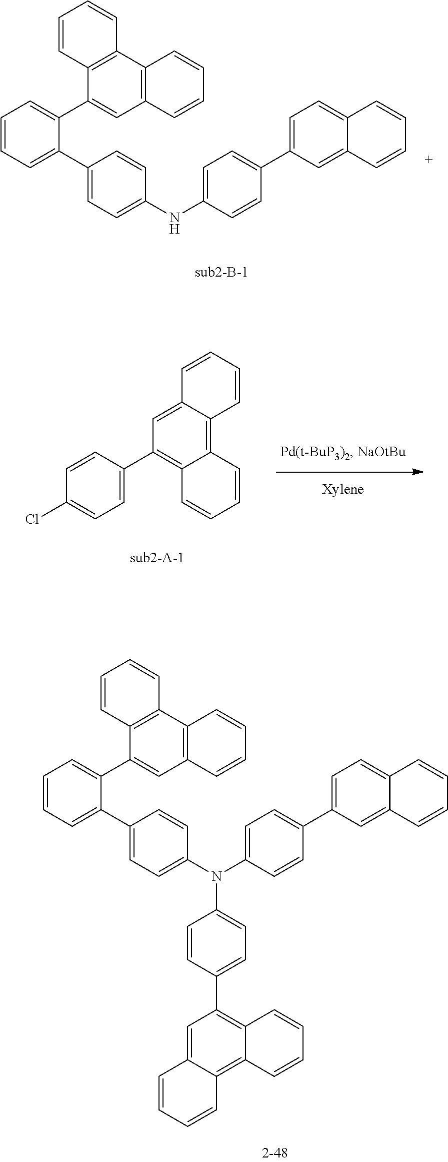 [SMILES: c1ccc(-c2cc3ccccc3c3ccccc23)c(-c2ccc(Nc3ccc(-c4ccc5ccccc5c4)cc3)cc2)c1 ; Clc1ccc(-c2cc3ccccc3c3ccccc23)cc1 ; c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1]

Compound sub2-B-1 (10 g, 18.3 mmol), Compound sub2-A-1 (5.3 g, 18.3 mmol) and sodium tert-butoxide (2.3 g, 23.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 7.7 g of Compound 2-48. (Yield: 53%, MS: [M+H]⁺=800)

### Preparation Example 2-49: Preparation of Compound 2-49

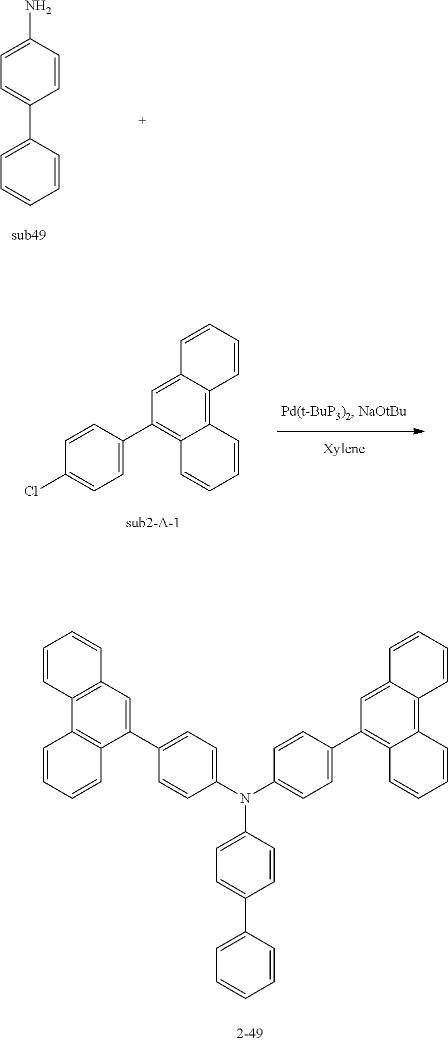 [SMILES: Nc1ccc(-c2ccccc2)cc1 ; Clc1ccc(-c2cc3ccccc3c3ccccc23)cc1 ; c1ccc(-c2ccc(N(c3ccc(-c4cc5ccccc5c5ccccc45)cc3)c3ccc(-c4cc5ccccc5c5ccccc45)cc3)cc2)cc1]

Compound sub49 (10 g, 59.1 mmol), Compound sub2-A-1 (35.8 g, 124.1 mmol) and sodium tert-butoxide (14.2 g, 147.7 mmol) were added to 200 ml of xylene under a nitrogen atmosphere, and the mixture was stirred and refluxed. Then, bis(tri-tert-butylphosphine)palladium(0) (0.6 g, 1.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 22.7 g of Compound 2-49. (Yield: 57%, MS: [M+H]⁺=674)

### Preparation Example 2-50: Preparation of Compound 2-50

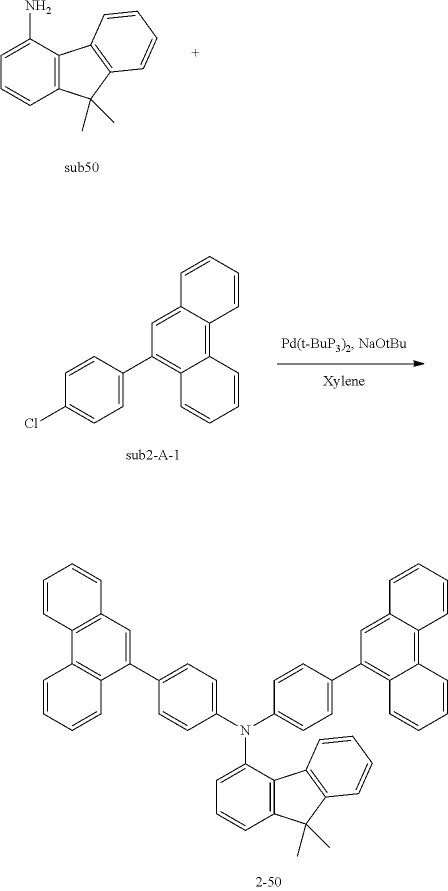 [SMILES: CC1(C)c2ccccc2-c2c(N)cccc21 ; Clc1ccc(-c2cc3ccccc3c3ccccc23)cc1 ; CC1(C)c2ccccc2-c2c(N(c3ccc(-c4cc5ccccc5c5ccccc45)cc3)c3ccc(-c4cc5ccccc5c5ccccc45)cc3)cccc21]

Compound sub50 (10 g, 47.8 mmol), Compound sub2-A-1 (29 g, 100.3 mmol) and sodium tert-butoxide (11.5 g, 119.5 mmol) were added to 200 ml of xylene under a nitrogen atmosphere, and the mixture was stirred and refluxed. Then, bis(tri-tert-butylphosphine)palladium(0) (0.5 g, 1 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 23.9 g of Compound 2-50. (Yield: 70%, MS: [M+H]⁺=714)

### Preparation Example 2-51: Preparation of Compound 2-51

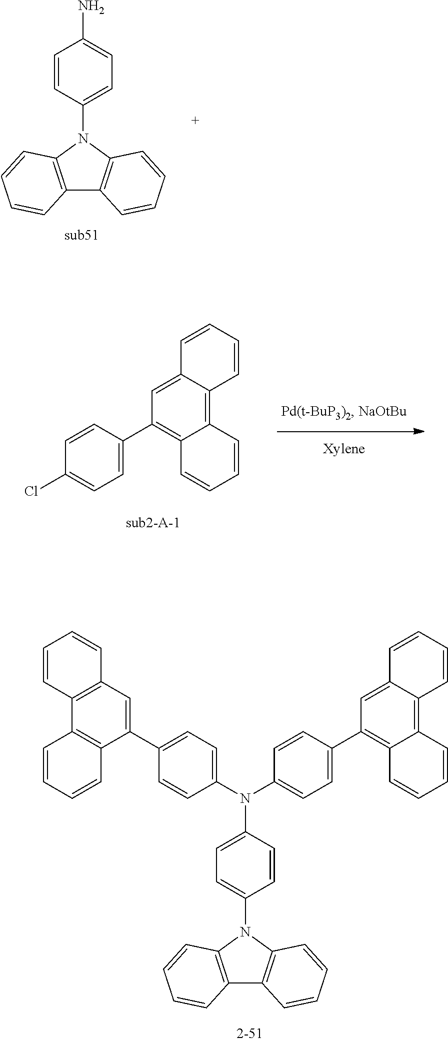 [SMILES: Nc1ccc(-n2c3ccccc3c3ccccc32)cc1 ; Clc1ccc(-c2cc3ccccc3c3ccccc23)cc1 ; c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12]

Compound sub51 (10 g, 38.7 mmol), Compound sub2-A-1 (23.5 g, 81.3 mmol) and sodium tert-butoxide (9.3 g, 96.8 mmol) were added to 200 ml of xylene under a nitrogen atmosphere, and the mixture was stirred and refluxed. Then, bis(tri-tert-butylphosphine)palladium(0) (0.4 g, 0.8 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 16.8 g of Compound 2-51. (Yield: 57%, MS: [M+H]⁺=763)

### Preparation Example 2-52: Preparation of Compound 2-52

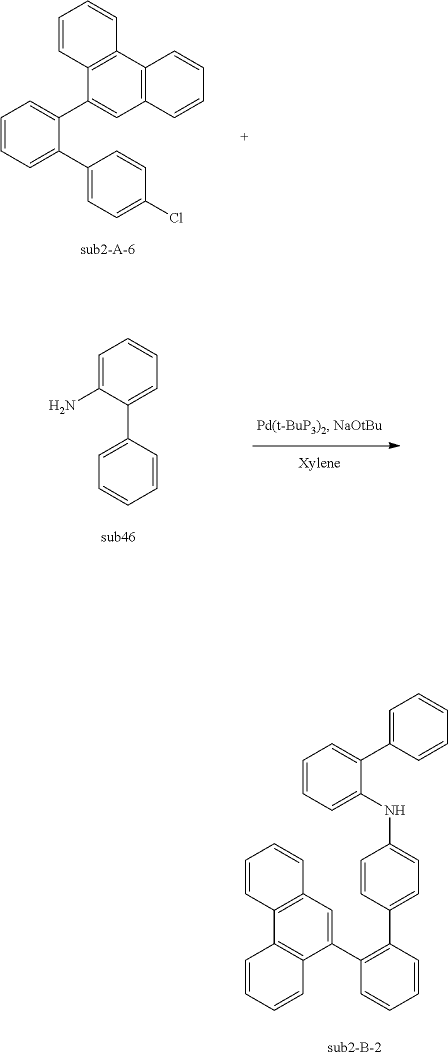 [SMILES: Clc1ccc(-c2ccccc2-c2cc3ccccc3c3ccccc23)cc1 ; Nc1ccccc1-c1ccccc1 ; c1ccc(-c2ccccc2Nc2ccc(-c3ccccc3-c3cc4ccccc4c4ccccc34)cc2)cc1]

Compound sub2-A-6 (10 g, 27.4 mmol), Compound sub46 (4.6 g, 27.4 mmol) and sodium tert-butoxide (2.9 g, 30.1 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 9.4 g of Compound sub2-B-2. (Yield: 69%, MS: [M+H]⁺=498)

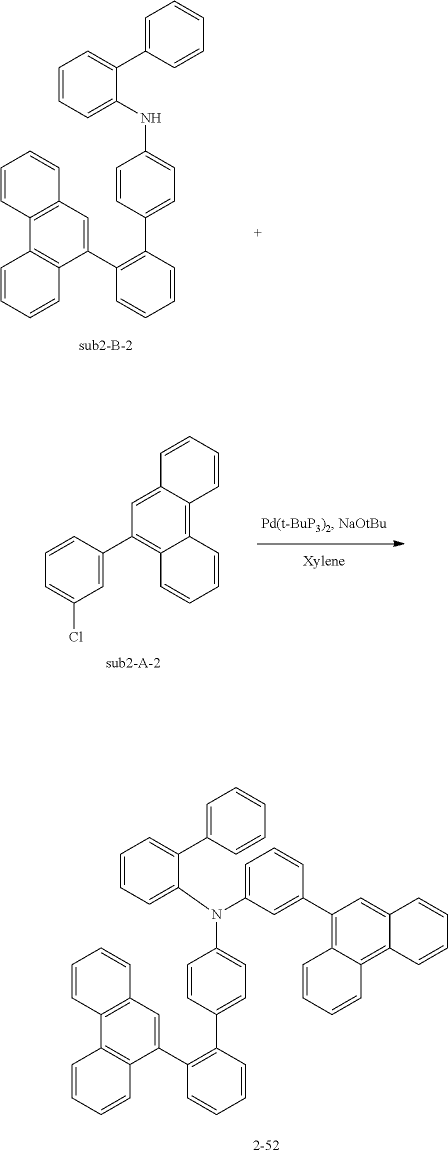 [SMILES: c1ccc(-c2ccccc2Nc2ccc(-c3ccccc3-c3cc4ccccc4c4ccccc34)cc2)cc1 ; Clc1cccc(-c2cc3ccccc3c3ccccc23)c1 ; c1ccc(-c2ccccc2N(c2ccc(-c3ccccc3-c3cc4ccccc4c4ccccc34)cc2)c2cccc(-c3cc4ccccc4c4ccccc34)c2)cc1]

Compound sub2-B-2 (10 g, 20.1 mmol), Compound sub2-A-2 (5.8 g, 20.1 mmol) and sodium tert-butoxide (2.5 g, 26.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 8.3 g of Compound 2-52. (Yield: 55%, MS: [M+H]⁺=750)

### Preparation Example 2-53: Preparation of Compound 2-53

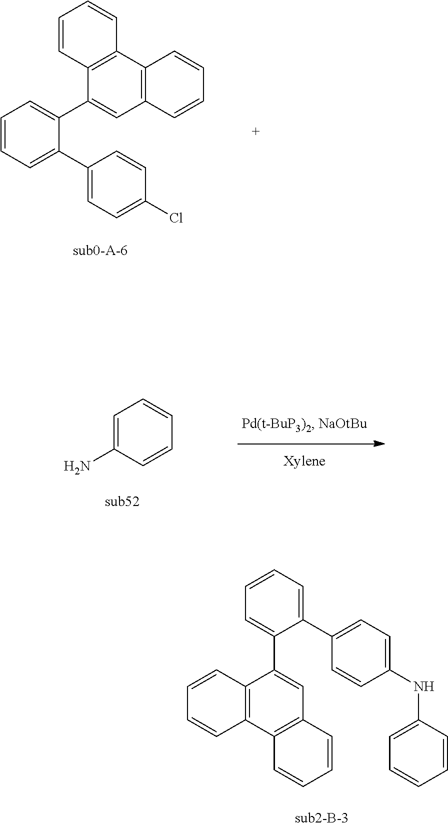 [SMILES: Clc1ccc(-c2ccccc2-c2cc3ccccc3c3ccccc23)cc1 ; Nc1ccccc1 ; c1ccc(Nc2ccc(-c3ccccc3-c3cc4ccccc4c4ccccc34)cc2)cc1]

Compound sub2-A-6 (10 g, 27.4 mmol), Compound sub52 (2.6 g, 27.4 mmol) and sodium tert-butoxide (2.9 g, 30.1 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 5.9 g of Compound sub2-B-3. (Yield: 51%, MS: [M+H]⁺=422)

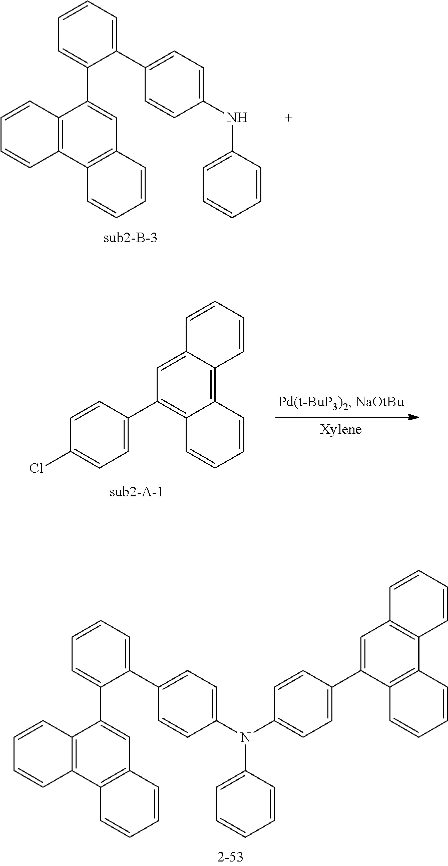 [SMILES: c1ccc(Nc2ccc(-c3ccccc3-c3cc4ccccc4c4ccccc34)cc2)cc1 ; Clc1ccc(-c2cc3ccccc3c3ccccc23)cc1 ; c1ccc(N(c2ccc(-c3ccccc3-c3cc4ccccc4c4ccccc34)cc2)c2ccc(-c3cc4ccccc4c4ccccc34)cc2)cc1]

Compound sub2-B-3 (10 g, 23.7 mmol), Compound sub2-A-1 (6.9 g, 23.7 mmol) and sodium tert-butoxide (3 g, 30.8 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 9.3 g of Compound 2-53. (Yield: 58%, MS: [M+H]⁺=674)

### Preparation Example 2-54: Preparation of Compound 2-54

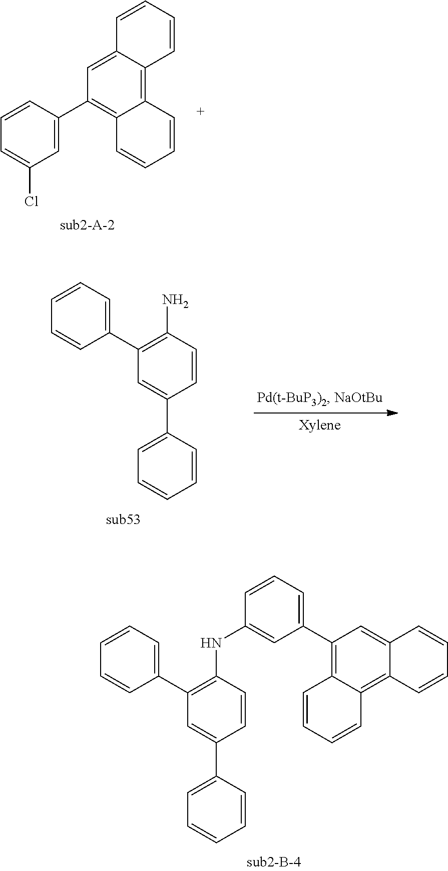 [SMILES: Clc1cccc(-c2cc3ccccc3c3ccccc23)c1 ; Nc1ccc(-c2ccccc2)cc1-c1ccccc1 ; c1ccc(-c2ccc(Nc3cccc(-c4cc5ccccc5c5ccccc45)c3)c(-c3ccccc3)c2)cc1]

Compound sub2-A-2 (10 g, 34.6 mmol), Compound sub53 (8.5 g, 34.6 mmol) and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.5 g of Compound sub2-B-4. (Yield: 67%, MS: [M+H]⁺=498)

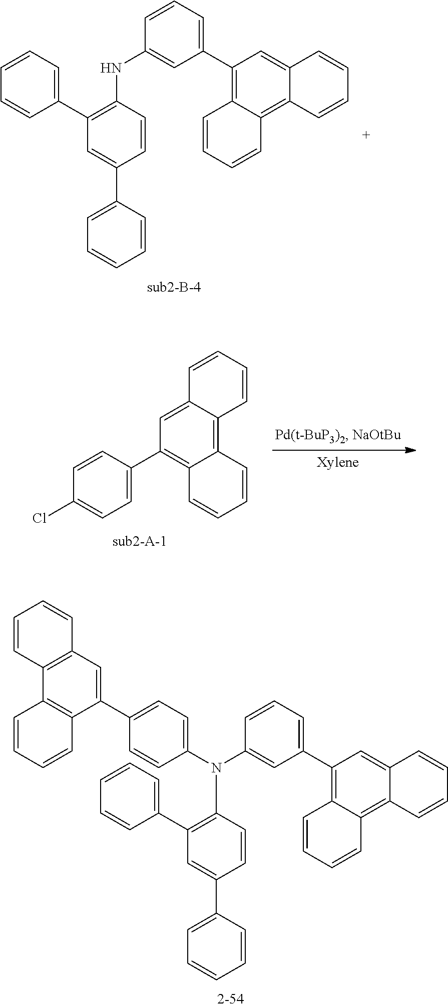 [SMILES: c1ccc(-c2ccc(Nc3cccc(-c4cc5ccccc5c5ccccc45)c3)c(-c3ccccc3)c2)cc1 ; Clc1ccc(-c2cc3ccccc3c3ccccc23)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1]

Compound sub2-B-4 (10 g, 20.1 mmol), Compound sub2-A-1 (5.8 g, 20.1 mmol) and sodium tert-butoxide (2.5 g, 26.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 7.5 g of Compound 2-54. (Yield: 50%, MS: [M+H]⁺=750)

### Preparation Example 2-55: Preparation of Compound 2-55

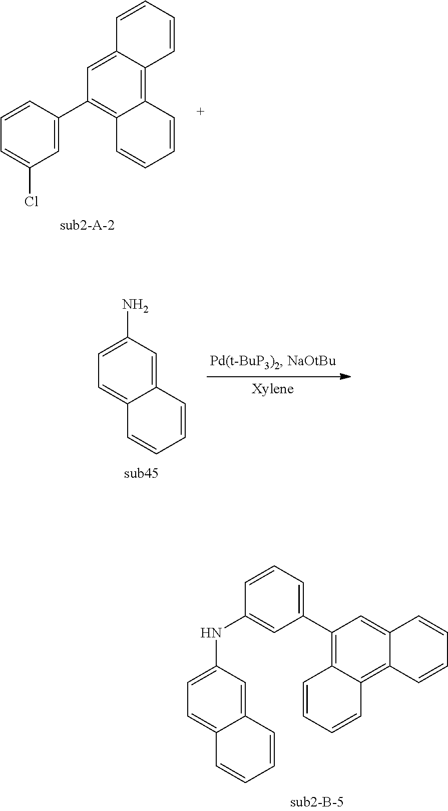 [SMILES: Clc1cccc(-c2cc3ccccc3c3ccccc23)c1 ; Nc1ccc2ccccc2c1 ; c1cc(Nc2ccc3ccccc3c2)cc(-c2cc3ccccc3c3ccccc23)c1]

Compound sub2-A-2 (10 g, 34.6 mmol), Compound sub45 (5 g, 34.6 mmol) and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 9.3 g of Compound sub2-B-5. (Yield: 68%, MS: [M+H]⁺=396)

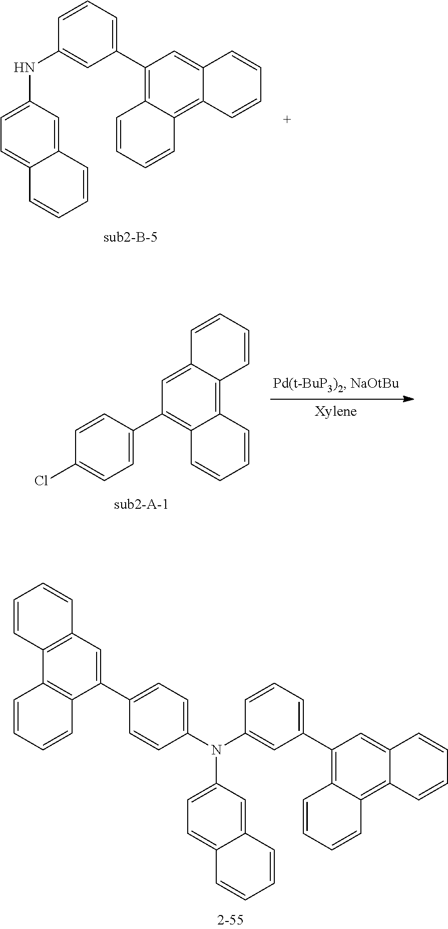 [SMILES: c1cc(Nc2ccc3ccccc3c2)cc(-c2cc3ccccc3c3ccccc23)c1 ; Clc1ccc(-c2cc3ccccc3c3ccccc23)cc1 ; c1cc(-c2cc3ccccc3c3ccccc23)cc(N(c2ccc(-c3cc4ccccc4c4ccccc34)cc2)c2ccc3ccccc3c2)c1]

Compound sub2-B-5 (10 g, 25.3 mmol), Compound sub2-A-1 (7.3 g, 25.3 mmol) and sodium tert-butoxide (3.2 g, 32.9 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 10 g of Compound 2-55. (Yield: 61%, MS: [M+H]⁺=648)

### Preparation Example 2-56: Preparation of Compound 2-56

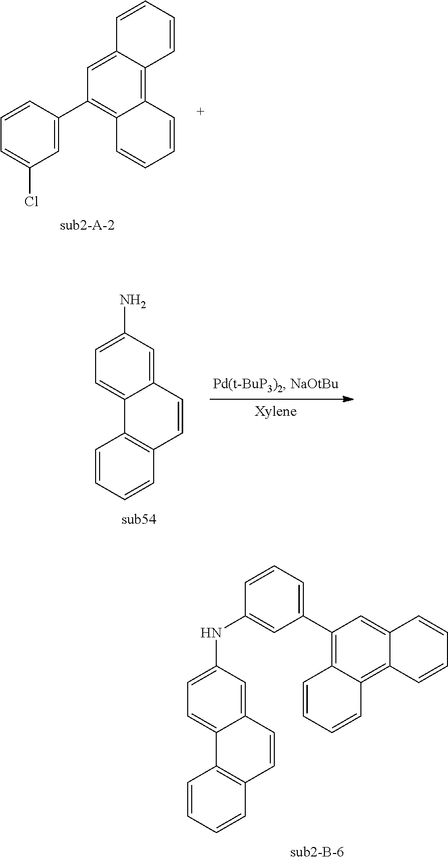 [SMILES: Clc1cccc(-c2cc3ccccc3c3ccccc23)c1 ; Nc1ccc2c(ccc3ccccc32)c1 ; c1cc(Nc2ccc3c(ccc4ccccc43)c2)cc(-c2cc3ccccc3c3ccccc23)c1]

Compound sub2-A-2 (10 g, 34.6 mmol), Compound sub54 (6.7 g, 34.6 mmol) and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 8.6 g of Compound sub2-B-6. (Yield: 56%, MS: [M+H]⁺=446)

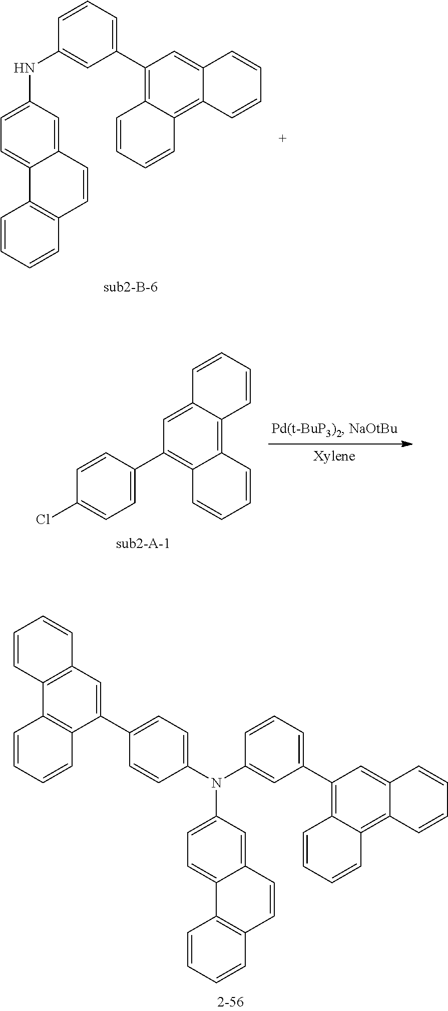 [SMILES: c1cc(Nc2ccc3c(ccc4ccccc43)c2)cc(-c2cc3ccccc3c3ccccc23)c1 ; Clc1ccc(-c2cc3ccccc3c3ccccc23)cc1 ; c1cc(-c2cc3ccccc3c3ccccc23)cc(N(c2ccc(-c3cc4ccccc4c4ccccc34)cc2)c2ccc3c(ccc4ccccc43)c2)c1]

Compound sub2-B-6 (10 g, 22.4 mmol), Compound sub2-A-1 (6.5 g, 22.4 mmol) and sodium tert-butoxide (2.8 g, 29.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 8.8 g of Compound 2-56. (Yield: 56%, MS: [M+H]⁺=698)

### Preparation Example 2-57: Preparation of Compound 2-57

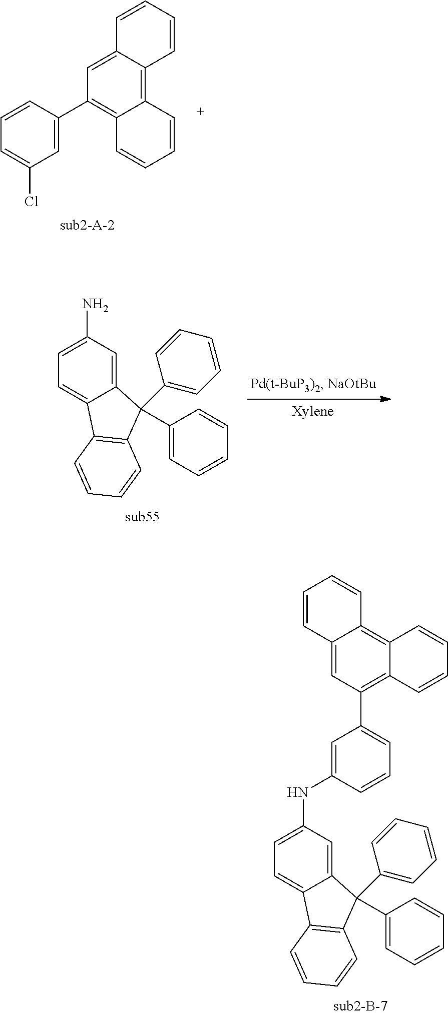 [SMILES: Clc1cccc(-c2cc3ccccc3c3ccccc23)c1 ; Nc1ccc2c(c1)C(c1ccccc1)(c1ccccc1)c1ccccc1-2 ; c1ccc(C2(c3ccccc3)c3ccccc3-c3ccc(Nc4cccc(-c5cc6ccccc6c6ccccc56)c4)cc32)cc1]

Compound sub2-A-2 (10 g, 34.6 mmol), Compound sub55 (11.5 g, 34.6 mmol) and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 13.2 g of Compound sub2-B-7. (Yield: 65%, MS: [M+H]⁺=586)

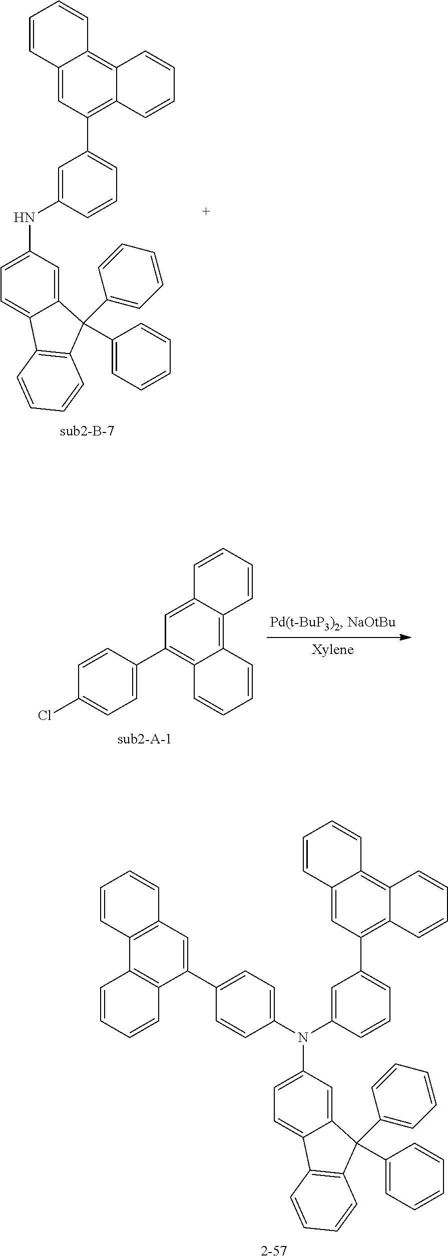 [SMILES: c1ccc(C2(c3ccccc3)c3ccccc3-c3ccc(Nc4cccc(-c5cc6ccccc6c6ccccc56)c4)cc32)cc1 ; Clc1ccc(-c2cc3ccccc3c3ccccc23)cc1 ; c1ccc(C2(c3ccccc3)c3ccccc3-c3ccc(N(c4ccc(-c5cc6ccccc6c6ccccc56)cc4)c4cccc(-c5cc6ccccc6c6ccccc56)c4)cc32)cc1]

Compound sub2-B-7 (10 g, 17.1 mmol), Compound sub2-A-1 (4.9 g, 17.1 mmol) and sodium tert-butoxide (2.1 g, 22.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 7.7 g of Compound 2-57. (Yield: 54%, MS: [M+H]⁺=838)

### Preparation Example 2-58: Preparation of Compound 2-58

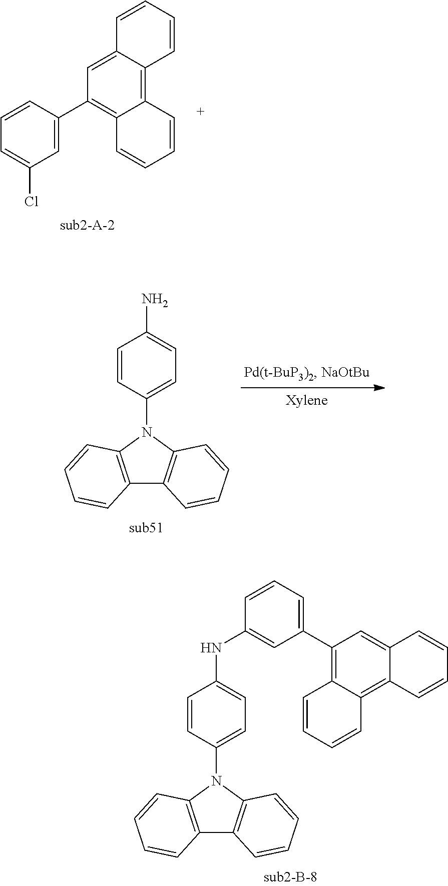 [SMILES: Clc1cccc(-c2cc3ccccc3c3ccccc23)c1 ; Nc1ccc(-n2c3ccccc3c3ccccc32)cc1 ; c1cc(Nc2ccc(-n3c4ccccc4c4ccccc43)cc2)cc(-c2cc3ccccc3c3ccccc23)c1]

Compound sub2-A-2 (10 g, 34.6 mmol), Compound sub51 (8.9 g, 34.6 mmol) and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 10.8 g of Compound sub2-B-8. (Yield: 61%, MS: [M+H]⁺=511)

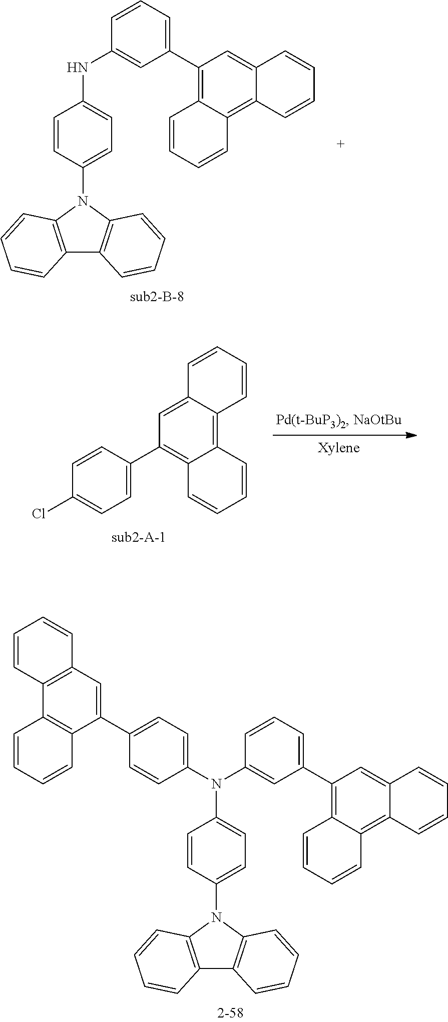 [SMILES: c1cc(Nc2ccc(-n3c4ccccc4c4ccccc43)cc2)cc(-c2cc3ccccc3c3ccccc23)c1 ; Clc1ccc(-c2cc3ccccc3c3ccccc23)cc1 ; c1cc(-c2cc3ccccc3c3ccccc23)cc(N(c2ccc(-c3cc4ccccc4c4ccccc34)cc2)c2ccc(-n3c4ccccc4c4ccccc43)cc2)c1]

Compound sub2-B-8 (10 g, 19.6 mmol), Compound sub2-A-1 (5.7 g, 19.6 mmol) and sodium tert-butoxide (2.4 g, 25.5 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 7.6 g of Compound 2-58. (Yield: 51%, MS: [M+H]⁺=763)

### Preparation Example 2-59: Preparation of Compound 2-59

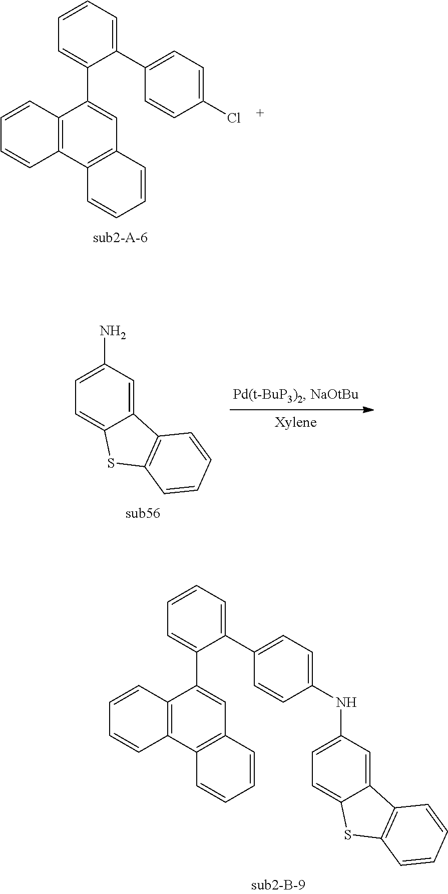 [SMILES: Clc1ccc(-c2ccccc2-c2cc3ccccc3c3ccccc23)cc1 ; Nc1ccc2sc3ccccc3c2c1 ; c1ccc(-c2cc3ccccc3c3ccccc23)c(-c2ccc(Nc3ccc4sc5ccccc5c4c3)cc2)c1]

Compound sub2-A-6 (10 g, 27.4 mmol), Compound sub56 (5.5 g, 27.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 7.5 g of Compound sub2-B-9. (Yield: 52%, MS: [M+H]⁺=528)

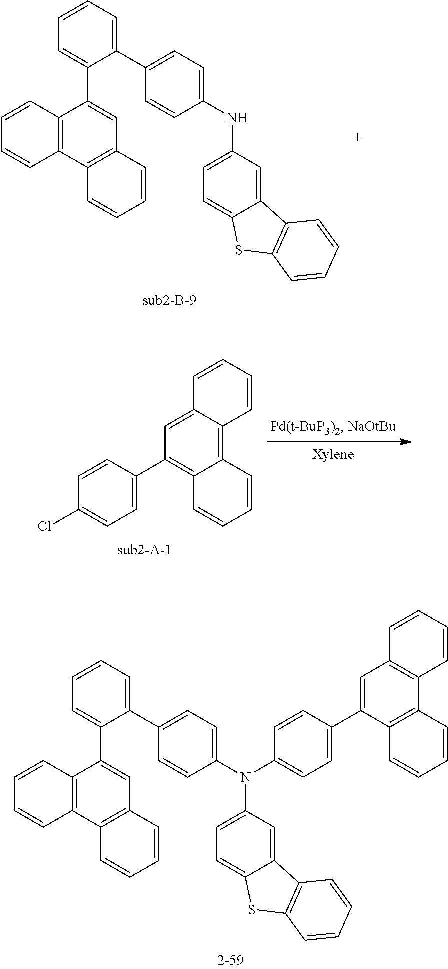 [SMILES: c1ccc(-c2cc3ccccc3c3ccccc23)c(-c2ccc(Nc3ccc4sc5ccccc5c4c3)cc2)c1 ; Clc1ccc(-c2cc3ccccc3c3ccccc23)cc1 ; c1ccc(-c2cc3ccccc3c3ccccc23)c(-c2ccc(N(c3ccc(-c4cc5ccccc5c5ccccc45)cc3)c3ccc4sc5ccccc5c4c3)cc2)c1]

Compound sub2-B-9 (10 g, 19 mmol), Compound sub2-A-1 (5.5 g, 19 mmol) and sodium tert-butoxide (2.4 g, 24.6 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 8.7 g of Compound 2-59. (Yield: 59%, MS: [M+H]⁺=780)

### Preparation Example 2-60: Preparation of Compound 2-60

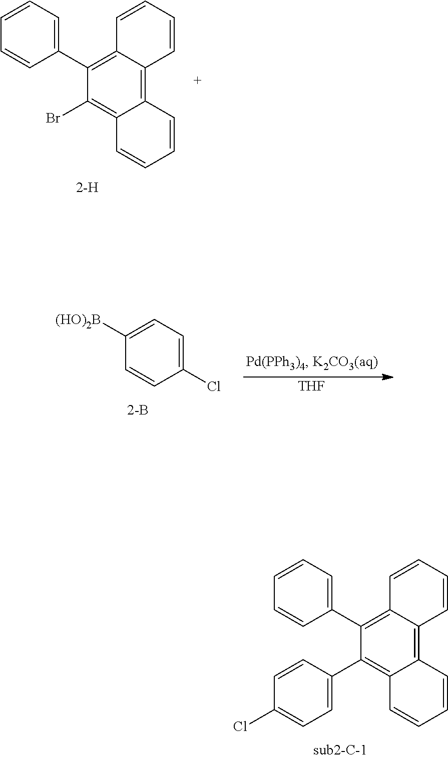 [SMILES: Brc1c(-c2ccccc2)c2ccccc2c2ccccc12 ; OB(O)c1ccc(Cl)cc1 ; Clc1ccc(-c2c(-c3ccccc3)c3ccccc3c3ccccc23)cc1 ; "THF"]

Compound 2-H (15 g, 45 mmol) and Compound 2-B (7.7 g, 49.5 mmol) were added to 300 ml of THE under a nitrogen atmosphere, and the mixture was stirred and refluxed. Then, potassium carbonate (12.4 g, 90 mmol) was dissolved in 37 ml of water, added thereto, and the mixture was sufficiently stirred and then tetrakis(triphenylphosphine)palladium(0) (1 g, 0.9 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.3 g of Compound sub2-C-1. (Yield: 75%, MS: [M+H]⁺=365)

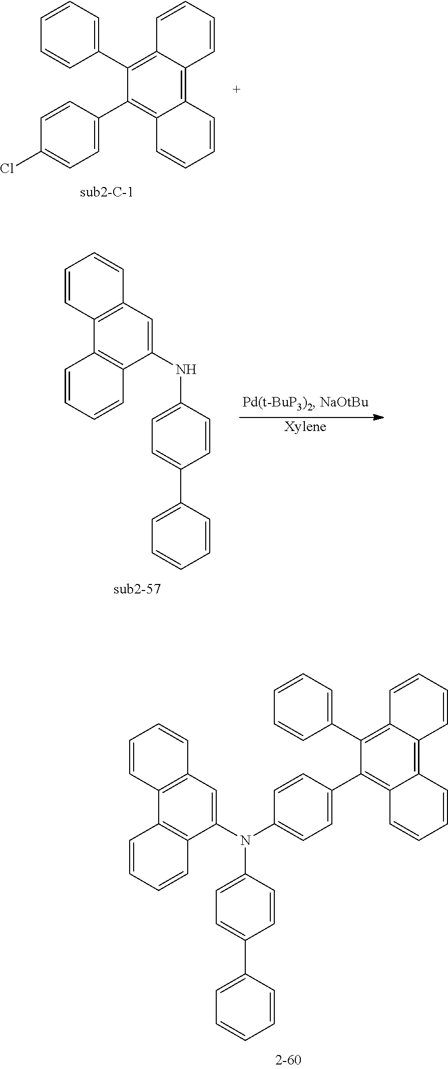 [SMILES: Clc1ccc(-c2c(-c3ccccc3)c3ccccc3c3ccccc23)cc1 ; c1ccc(-c2ccc(Nc3cc4ccccc4c4ccccc34)cc2)cc1 ; "Xylene" ; c1ccc(-c2ccc(N(c3ccc(-c4c(-c5ccccc5)c5ccccc5c5ccccc45)cc3)c3cc4ccccc4c4ccccc34)cc2)cc1]

Compound sub2-C-1 (10 g, 27.4 mmol), Compound sub2-57 (9.5 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.7 g of Compound 2-60. (Yield: 69%, MS: [M+H]⁺=674)

### Preparation Example 2-61: Preparation of Compound 2-61

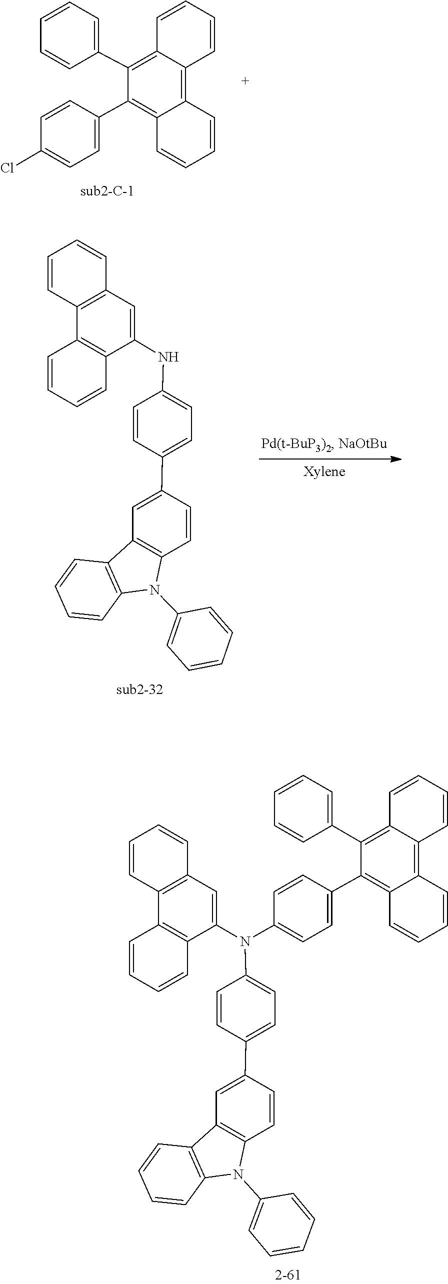 [SMILES: Clc1ccc(-c2c(-c3ccccc3)c3ccccc3c3ccccc23)cc1 ; c1ccc(-n2c3ccccc3c3cc(-c4ccc(Nc5cc6ccccc6c6ccccc56)cc4)ccc32)cc1 ; c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1]

Compound sub2-C-1 (10 g, 27.4 mmol), Compound sub2-32 (14 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.6 g of Compound 2-61. (Yield: 55%, MS: [M+H]⁺=839)

### Preparation Example 2-62: Preparation of Compound 2-62

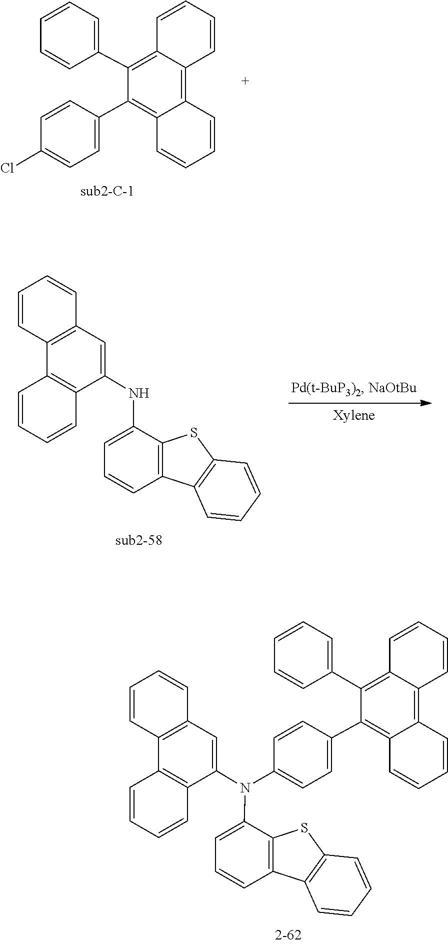 [SMILES: Clc1ccc(-c2c(-c3ccccc3)c3ccccc3c3ccccc23)cc1 ; c1ccc2c(c1)cc(Nc1cccc3c1sc1ccccc13)c1ccccc12 ; c1ccc(-c2c(-c3ccc(N(c4cc5ccccc5c5ccccc45)c4cccc5c4sc4ccccc45)cc3)c3ccccc3c3ccccc23)cc1]

Compound sub2-C-1 (10 g, 27.4 mmol), Compound sub2-58 (10.3 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.5 g of Compound 2-62. (Yield: 65%, MS: [M+H]⁺=704)

### Preparation Example 2-63: Preparation of Compound 2-63

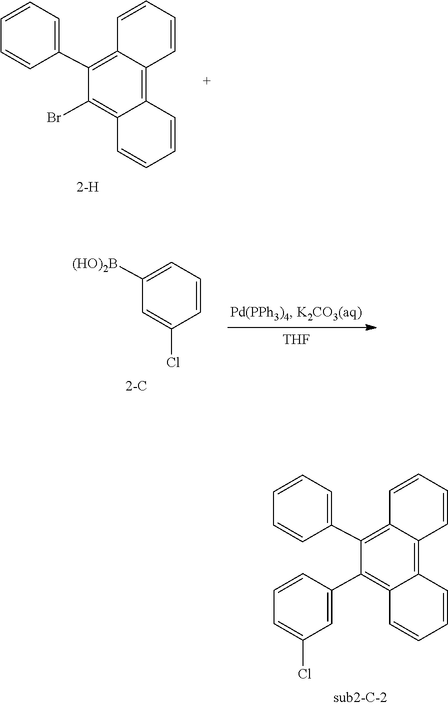 [SMILES: Brc1c(-c2ccccc2)c2ccccc2c2ccccc12 ; OB(O)c1cccc(Cl)c1 ; Clc1cccc(-c2c(-c3ccccc3)c3ccccc3c3ccccc23)c1 ; "THF"]

Compound 2-H (15 g, 45 mmol) and Compound 2-C (7.7 g, 49.5 mmol) were added to 300 ml of THE under a nitrogen atmosphere, and the mixture was stirred and refluxed. Then, potassium carbonate (12.4 g, 90 mmol) was dissolved in 37 ml of water, added thereto, and the mixture was sufficiently stirred and then tetrakis(triphenylphosphine)palladium(0) (1 g, 0.9 mmol) was added. After 11-hour reaction, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give 12.3 g of Compound sub2-C-2. (Yield: 75%, MS: [M+H]⁺=365)

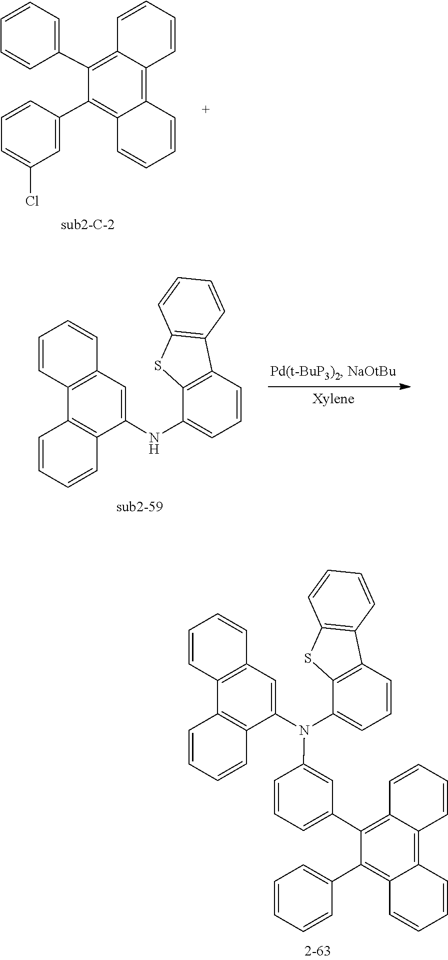 [SMILES: Clc1cccc(-c2c(-c3ccccc3)c3ccccc3c3ccccc23)c1 ; c1ccc2c(c1)cc(Nc1cccc3c1sc1ccccc13)c1ccccc12 ; c1ccc(-c2c(-c3cccc(N(c4cc5ccccc5c5ccccc45)c4cccc5c4sc4ccccc45)c3)c3ccccc3c3ccccc23)cc1]

Compound sub2-C-2 (10 g, 27.4 mmol), Compound sub2-59 (10.3 g, 27.4 mmol) and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 13.5 g of Compound 2-63. (Yield: 70%, MS: [M+H]⁺=704)

### Preparation Example 2-64: Preparation of Compound 2-64

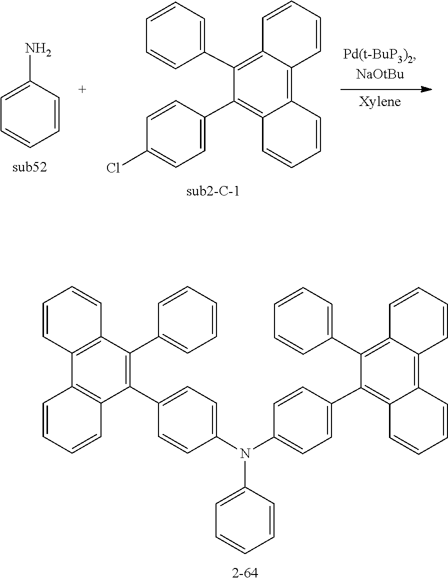 [SMILES: Nc1ccccc1 ; Clc1ccc(-c2c(-c3ccccc3)c3ccccc3c3ccccc23)cc1 ; c1ccc(-c2c(-c3ccc(N(c4ccccc4)c4ccc(-c5c(-c6ccccc6)c6ccccc6c6ccccc56)cc4)cc3)c3ccccc3c3ccccc23)cc1]

Compound sub52 (10 g, 107.4 mmol), Compound sub2-C-1 (82.3 g, 225.5 mmol) and sodium tert-butoxide (25.8 g, 268.4 mmol) were added to 200 ml of xylene under a nitrogen atmosphere, and the mixture was stirred and refluxed. Then, bis(tri-tert-butylphosphine)palladium(0) (1.1 g, 2.1 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 41 g of Compound 2-64. (Yield: 51%, MS: [M+H]⁺=750)

### Preparation Example 2-65: Preparation of Compound 2-65

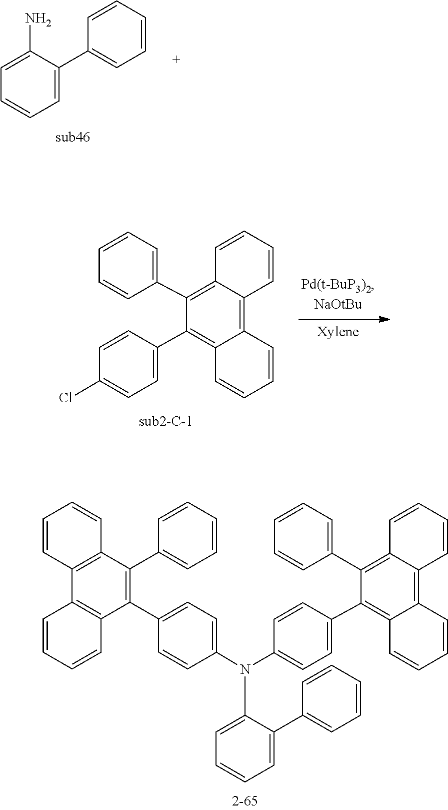 [SMILES: Nc1ccccc1-c1ccccc1 ; Clc1ccc(-c2c(-c3ccccc3)c3ccccc3c3ccccc23)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1]

Compound sub46 (10 g, 59.1 mmol), Compound sub2-C-1 (45.3 g, 124.1 mmol) and sodium tert-butoxide (14.2 g, 147.7 mmol) were added to 200 ml of xylene under a nitrogen atmosphere, and the mixture was stirred and refluxed. Then, bis(tri-tert-butylphosphine)palladium(0) (0.6 g, 1.2 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 31.2 g of Compound 2-65. (Yield: 64%, MS: [M+H]⁺=826)

### Preparation Example 2-66: Preparation of Compound 2-66

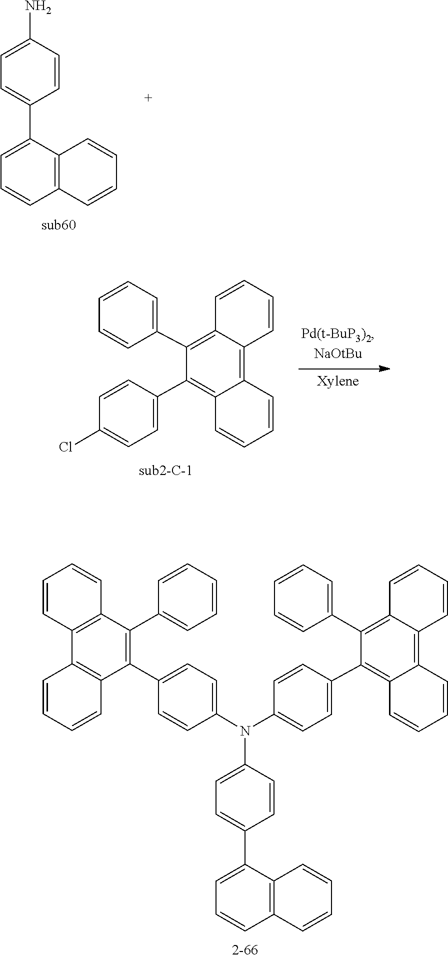 [SMILES: Nc1ccc(-c2cccc3ccccc23)cc1 ; Clc1ccc(-c2c(-c3ccccc3)c3ccccc3c3ccccc23)cc1 ; c1ccc(-c2c(-c3ccc(N(c4ccc(-c5cccc6ccccc56)cc4)c4ccc(-c5c(-c6ccccc6)c6ccccc6c6ccccc56)cc4)cc3)c3ccccc3c3ccccc23)cc1]

Compound sub60 (10 g, 45.6 mmol), Compound sub2-C-1 (34.9 g, 95.8 mmol) and sodium tert-butoxide (11 g, 114 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 26.7 g of Compound 2-66. (Yield: 67%, MS: [M+H]⁺=876)

### Preparation Example 2-67: Preparation of Compound 2-67

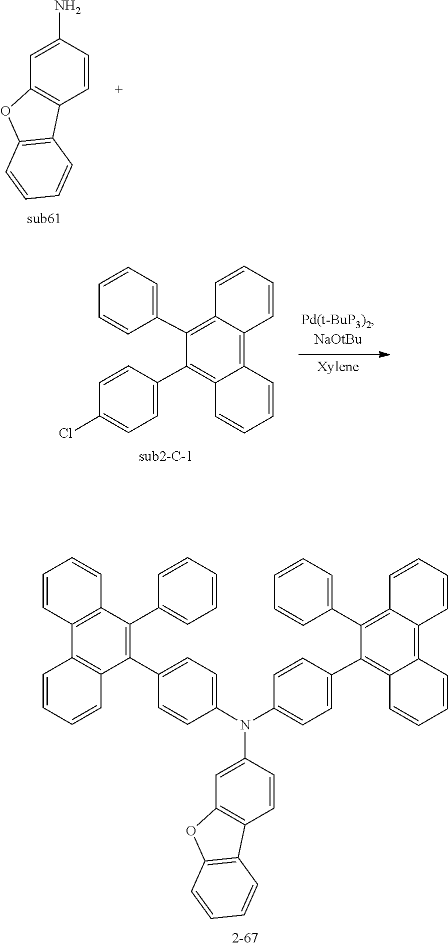 [SMILES: Nc1ccc2c(c1)oc1ccccc12 ; Clc1ccc(-c2c(-c3ccccc3)c3ccccc3c3ccccc23)cc1 ; c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1]

Compound sub61 (10 g, 54.6 mmol), Compound sub2-C-1 (41.8 g, 114.6 mmol) and sodium tert-butoxide (13.1 g, 136.5 mmol) were added to 200 ml of xylene under a nitrogen atmosphere, and the mixture was stirred and refluxed. Then, bis(tri-tert-butylphosphine)palladium(0) (0.6 g, 1.1 mmol) was added thereto. After 5 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 32.1 g of Compound 2-67. (Yield: 70%, MS: [M+H]⁺=840)

### Preparation Example 2-68: Preparation of Compound 2-68

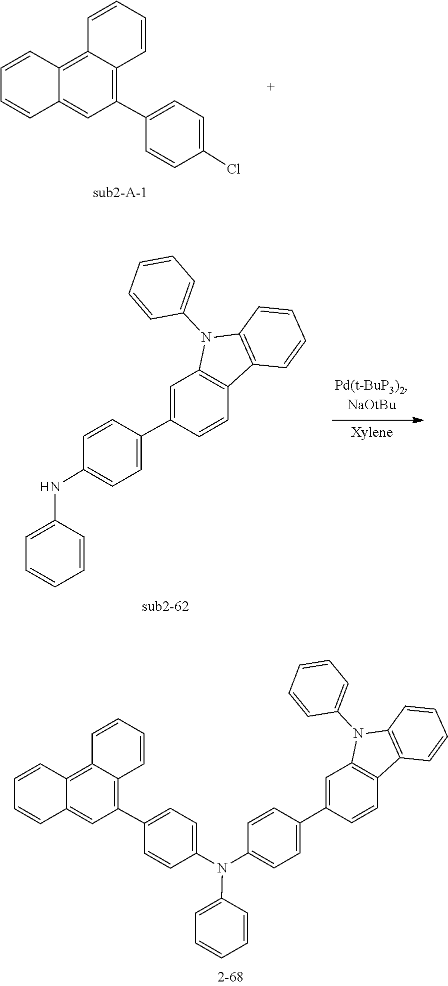 [SMILES: Clc1ccc(-c2cc3ccccc3c3ccccc23)cc1 ; c1ccc(Nc2ccc(-c3ccc4c5ccccc5n(-c5ccccc5)c4c3)cc2)cc1 ; c1ccc(N(c2ccc(-c3ccc4c5ccccc5n(-c5ccccc5)c4c3)cc2)c2ccc(-c3cc4ccccc4c4ccccc34)cc2)cc1]

Compound sub2-A-1 (10 g, 34.6 mmol), Compound sub2-62 (15.6 g, 38.1 mmol) and potassium phosphate (22.1 g, 103.9 mmol) were added to 200 ml of xylene under a nitrogen atmosphere, and the mixture was stirred and refluxed. Then, bis(tri-tert-butylphosphine)palladium(0) (0.4 g, 0.7 mmol) was added thereto. After 2 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.6 g of Compound 2-68. (Yield: 55%, MS: [M+H]⁺=663)

### Preparation Example 2-69: Preparation of Compound 2-69

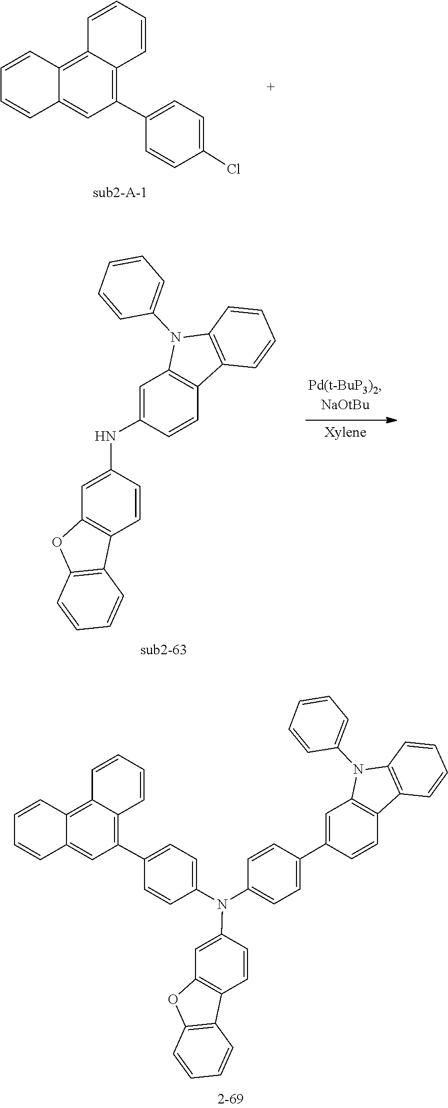 [SMILES: Clc1ccc(-c2cc3ccccc3c3ccccc23)cc1 ; c1ccc(-n2c3ccccc3c3ccc(Nc4ccc5c(c4)oc4ccccc45)cc32)cc1 ; c1ccc(-n2c3ccccc3c3ccc(-c4ccc(N(c5ccc(-c6cc7ccccc7c7ccccc67)cc5)c5ccc6c(c5)oc5ccccc56)cc4)cc32)cc1]

Compound sub2-A-1 (10 g, 34.6 mmol), Compound sub2-63 (16.2 g, 38.1 mmol) and potassium phosphate (22.1 g, 103.9 mmol) were added to 200 ml of xylene under a nitrogen atmosphere, and the mixture was stirred and refluxed. Then, bis(tri-tert-butylphosphine)palladium(0) (0.4 g, 0.7 mmol) was added thereto. After 2 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 12.6 g of Compound 2-69. (Yield: 54%, MS: [M+H]⁺=677)

### Preparation Example 2-70: Preparation of Compound 2-70

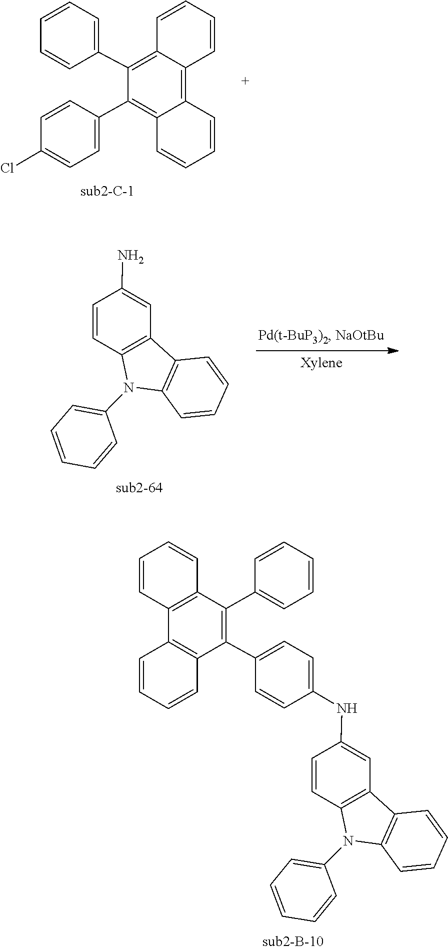 [SMILES: Clc1ccc(-c2c(-c3ccccc3)c3ccccc3c3ccccc23)cc1 ; Nc1ccc2c(c1)c1ccccc1n2-c1ccccc1 ; c1ccc(-c2c(-c3ccc(Nc4ccc5c(c4)c4ccccc4n5-c4ccccc4)cc3)c3ccccc3c3ccccc23)cc1]

Compound sub2-C-1 (10 g, 27.4 mmol), Compound sub2-64 (7.8 g, 30.1 mmol) and potassium phosphate (17.5 g, 82.2 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 2 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 11.2 g of Compound sub2-B-10. (Yield: 70%, MS: [M+H]⁺=587)

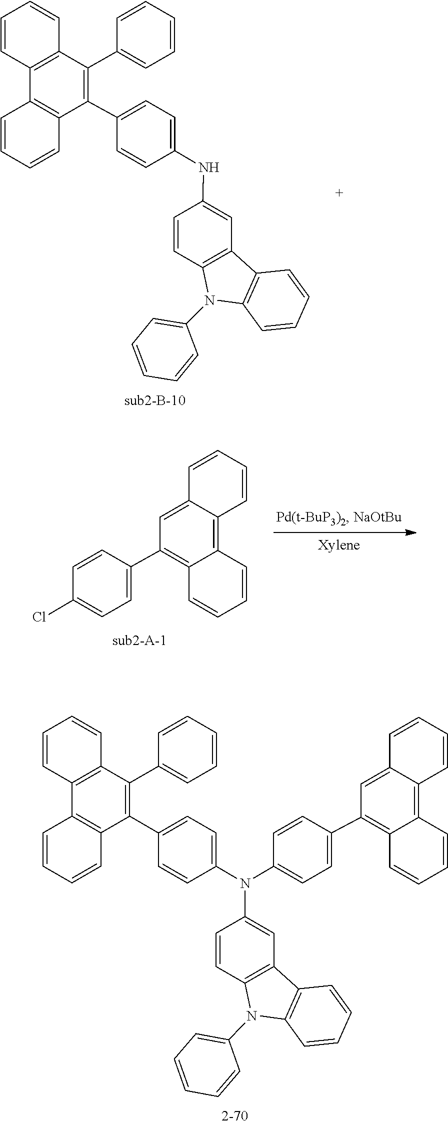 [SMILES: c1ccc(-c2c(-c3ccc(Nc4ccc5c(c4)c4ccccc4n5-c4ccccc4)cc3)c3ccccc3c3ccccc23)cc1 ; Clc1ccc(-c2cc3ccccc3c3ccccc23)cc1 ; c1ccc(-c2c(-c3ccc(N(c4ccc(-c5cc6ccccc6c6ccccc56)cc4)c4ccc5c(c4)c4ccccc4n5-c4ccccc4)cc3)c3ccccc3c3ccccc23)cc1]

Compound sub2-B-10 (10 g, 17 mmol), Compound sub2-A-1 (5.4 g, 18.7 mmol) and potassium phosphate (10.9 g, 51.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 3 hours, when the reaction was completed, the reaction mixture was cooled to room temperature and the solvent was removed under reduced pressure. Then, the compound was completely dissolved again in chloroform, washed twice with water, and then the organic layer was separated, treated with anhydrous magnesium sulfate, then filtered, and the filtrate was distilled under reduced pressure. The concentrated compound was purified by silica gel column chromatography to give 8.7 g of Compound 2-70. (Yield: 61%, MS: [M+H]⁺=839)

### EXAMPLES AND COMPARATIVE EXAMPLES

### Comparative Example 1

A glass substrate on which a thin film of ITO (indium tin oxide) was coated in a thickness of 1000 Å was put into distilled water containing a detergent dissolved therein and ultrasonically washed. In this case, the detergent used was a product commercially available from Fischer Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was formed as a hole injection layer, but the following compound A-1 was p-doped in an amount of 1.5% by weight. The following compound HT-1 was vacuum deposited on the hole injection layer to form a hole transport layer with a film thickness of 800 Å. Then, the following compound EB-1 was vacuum deposited to a film thickness of 150 Å on the hole transport layer to form an electron blocking layer. Then, the following compound 1-2 and the following compound Dp-7 were vacuum deposited in a weight ratio of 98:2 on the EB-1 deposited film to form a red light emitting layer with a thickness of 400 Å. The following compound HB-1 was vacuum deposited to a thickness of 30 Å on the light emitting layer to form a hole blocking layer. Then, the following compound ET-1 and the following compound LiQ were vacuum deposited in a ratio of 2:1 on the hole blocking layer to form an electron injection and transport layer with a thickness of 300 Å. Lithium fluoride (LiF) and aluminum were sequentially deposited to have a thickness of 12 Å and 1000 Å, respectively, on the electron injection and transport layer to form a cathode.

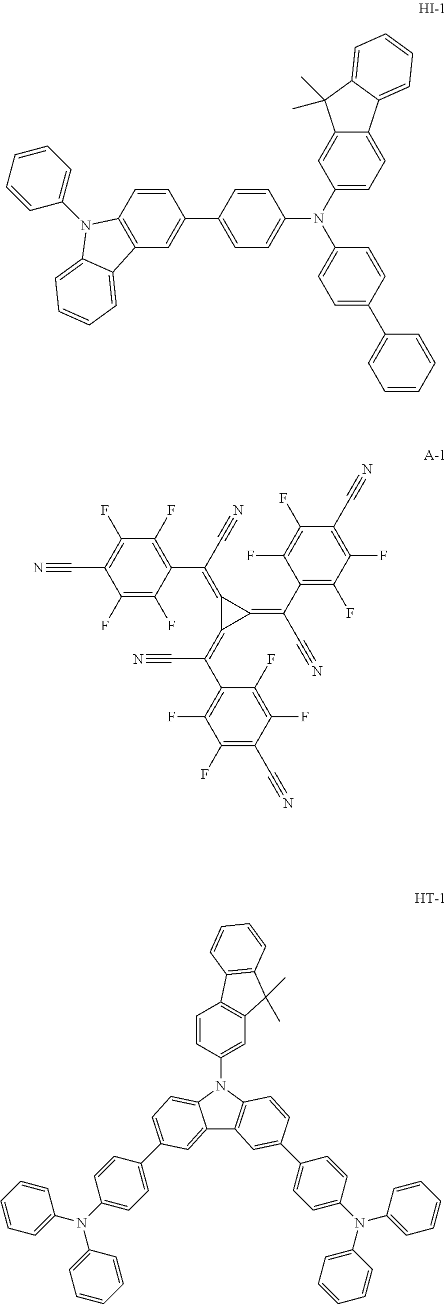 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(-n3c4ccc(-c5ccc(N(c6ccccc6)c6ccccc6)cc5)cc4c4cc(-c5ccc(N(c6ccccc6)c6ccccc6)cc5)ccc43)cc21]

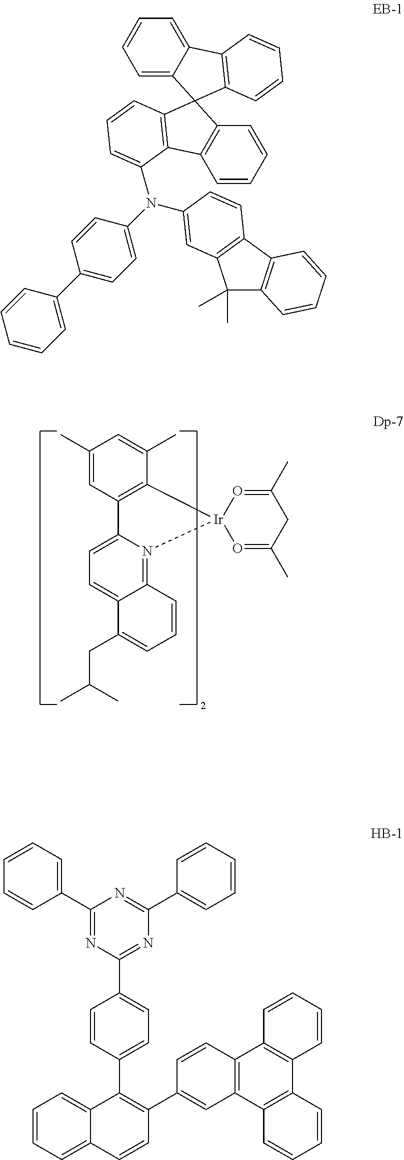

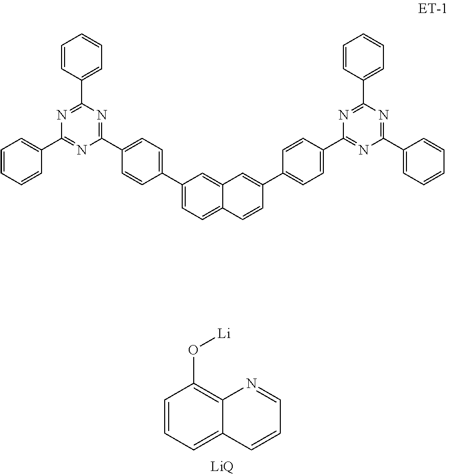

In the above-mentioned processes, the deposition rates of the organic materials were maintained at 0.4 to 0.7 Å/sec, the deposition rates of lithium fluoride and the aluminum of the cathode were maintained at 0.3 Å/sec and 2 Å/sec, respectively, and the degree of vacuum during the deposition was maintained at 2*10⁻⁷ to 5*10⁻⁶ torr.

### Examples 1 to 155

The organic light emitting devices were manufactured in the same manner as in Comparative Example 1, except that instead of Compound 1-2 as a host, the first host and the second host listed in Table 1 below were co-deposited at a weight ratio of 1:1 and used.

### Comparative Examples 2 to 61

The organic light emitting devices were manufactured in the same manner as in Comparative Example 1, except that the host material shown in Table 2 was used instead of Compound 1-2 as a host, and the compound shown in Table 2 below was used instead of Compound EB-1 as the electron blocking layer material.

### Comparative Examples 62 to 121

The organic light emitting devices were manufactured in the same manner as in Comparative Example 1, except that instead of Compound 1-2 as a host, the first host and the second host listed in Table 3 below were co-deposited at a weight ratio of 1:1 and used.

Compound B-1 to Compound B-12 in Table 3 are as follows:

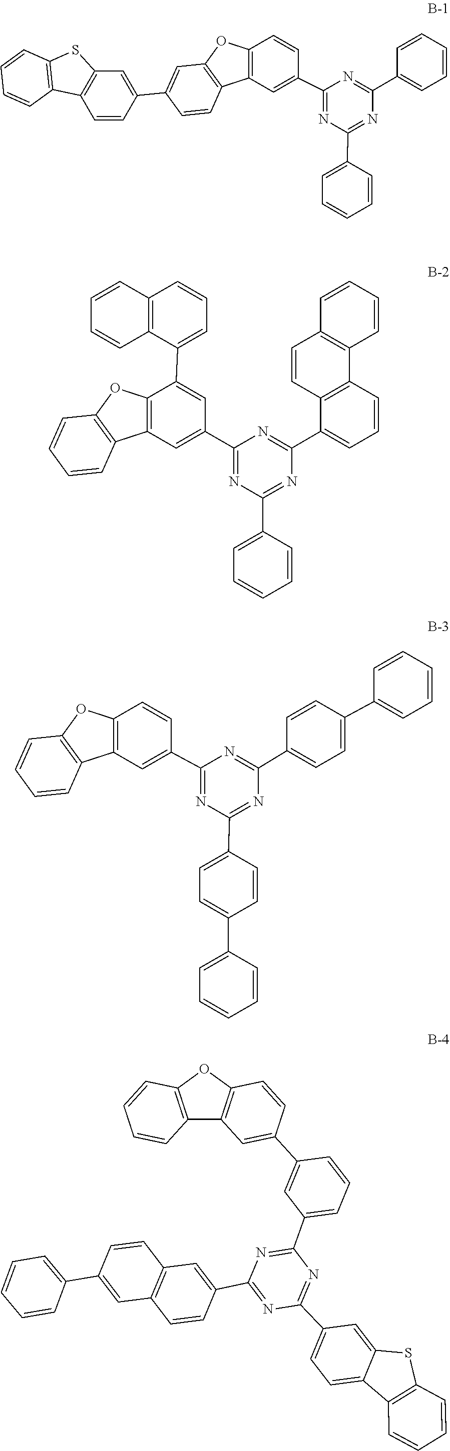

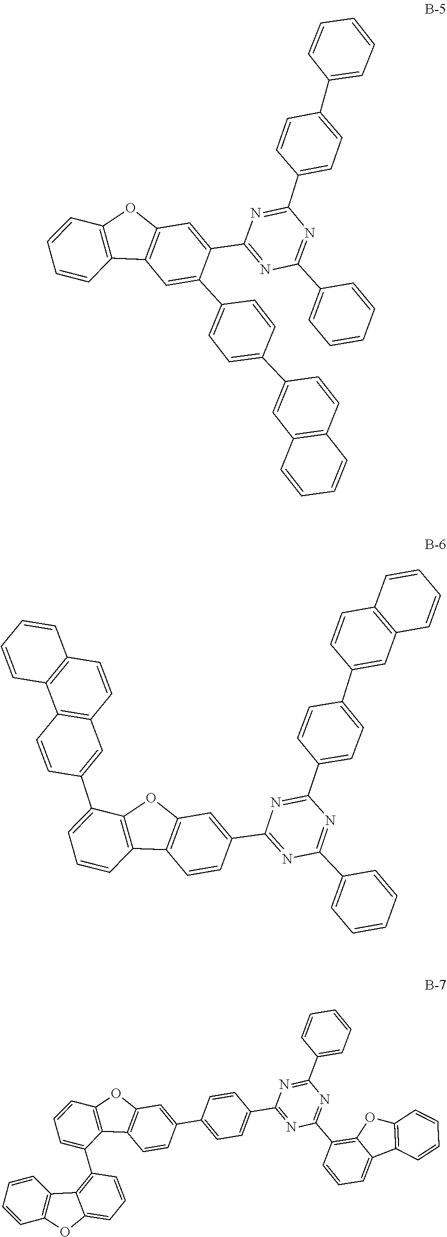 [SMILES: c1ccc(-c2ccc(-c3nc(-c4ccccc4)nc(-c4cc5oc6ccccc6c5cc4-c4ccc(-c5ccc6ccccc6c5)cc4)n3)cc2)cc1 ; c1ccc(-c2nc(-c3ccc(-c4ccc5ccccc5c4)cc3)nc(-c3ccc4c(c3)oc3c(-c5ccc6c(ccc7ccccc76)c5)cccc34)n2)cc1 ; c1ccc(-c2nc(-c3ccc(-c4ccc5c(c4)oc4cccc(-c6cccc7oc8ccccc8c67)c45)cc3)nc(-c3cccc4c3oc3ccccc34)n2)cc1]

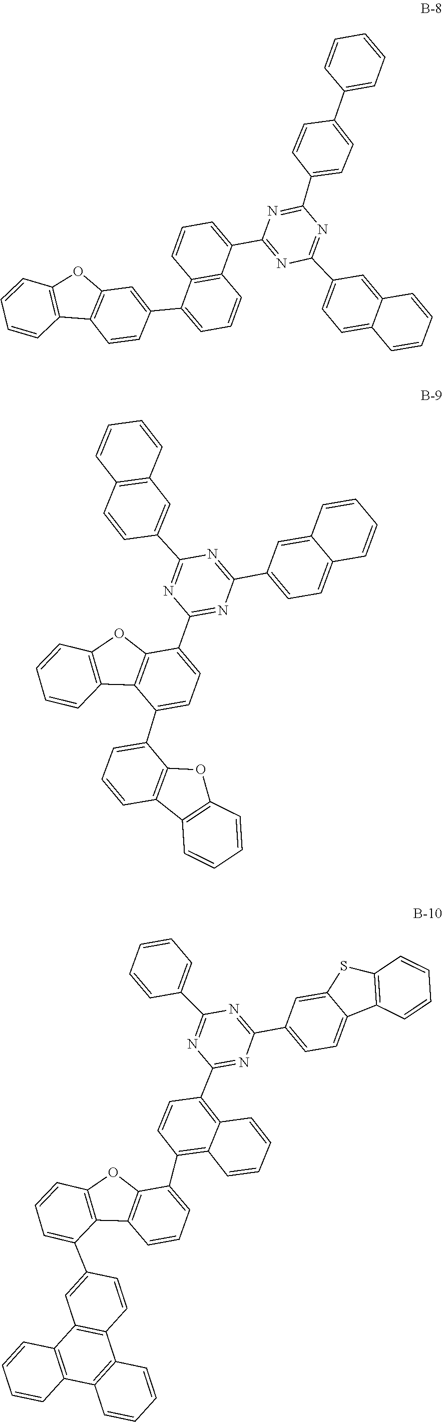 [SMILES: c1ccc(-c2ccc(-c3nc(-c4ccc5ccccc5c4)nc(-c4cccc5c(-c6ccc7c(c6)oc6ccccc67)cccc45)n3)cc2)cc1 ; c1ccc2cc(-c3nc(-c4ccc5ccccc5c4)nc(-c4ccc(-c5cccc6c5oc5ccccc56)c5c4oc4ccccc45)n3)ccc2c1 ; c1ccc(-c2nc(-c3ccc4c(c3)sc3ccccc34)nc(-c3ccc(-c4cccc5c4oc4cccc(-c6ccc7c8ccccc8c8ccccc8c7c6)c45)c4ccccc34)n2)cc1]

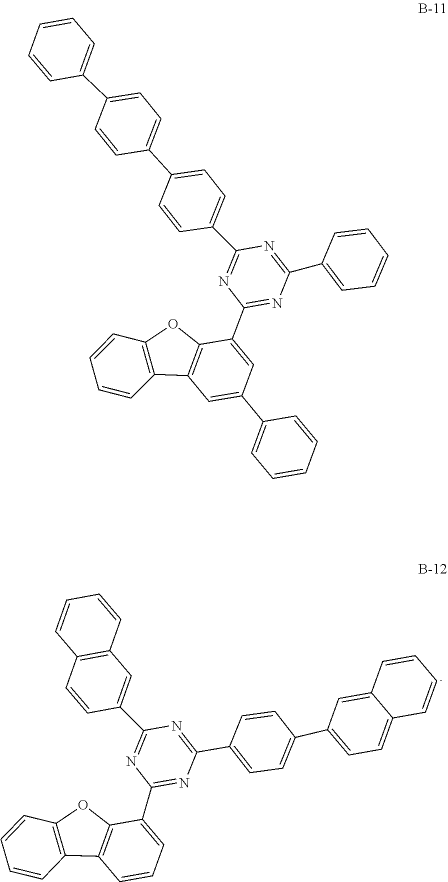 [SMILES: c1ccc(-c2ccc(-c3ccc(-c4nc(-c5ccccc5)nc(-c5cc(-c6ccccc6)cc6c5oc5ccccc56)n4)cc3)cc2)cc1 ; c1ccc2cc(-c3ccc(-c4nc(-c5ccc6ccccc6c5)nc(-c5cccc6c5oc5ccccc56)n4)cc3)ccc2c1]

### Comparative Examples 122 to 227

The organic light emitting devices were manufactured in the same manner as in Comparative Example 1, except that instead of Compound 1-2 as a host, the first host and the second host listed in Table 4 below were co-deposited at a weight ratio of 1:1 and used.

Compound C-1 to Compound C-12 in Table 4 are as follows:

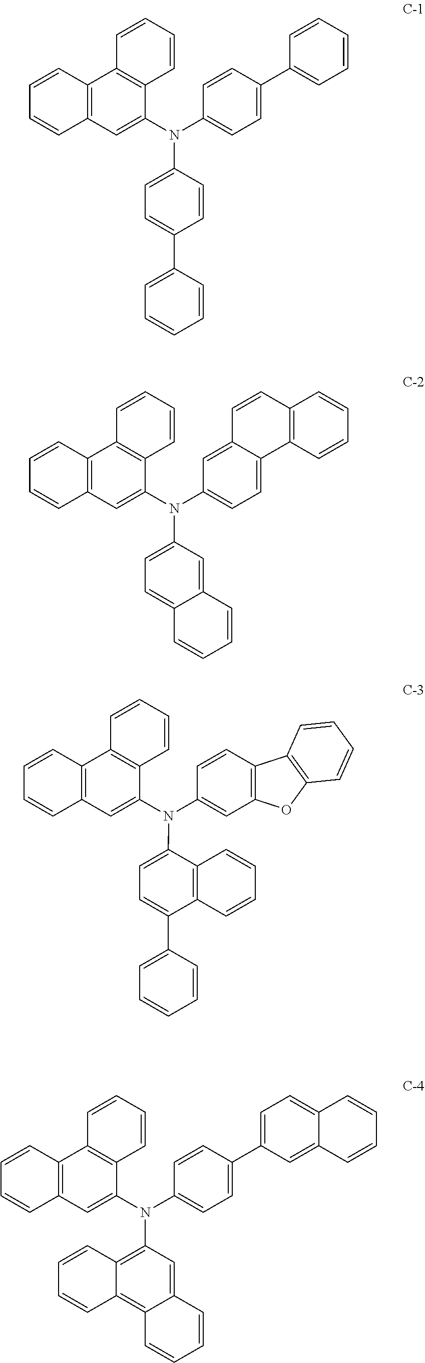 [SMILES: c1ccc(-c2ccc(N(c3ccc(-c4ccccc4)cc3)c3cc4ccccc4c4ccccc34)cc2)cc1 ; c1ccc2cc(N(c3ccc4c(ccc5ccccc54)c3)c3cc4ccccc4c4ccccc34)ccc2c1 ; c1ccc(-c2ccc(N(c3ccc4c(c3)oc3ccccc34)c3cc4ccccc4c4ccccc34)c3ccccc23)cc1 ; c1ccc2cc(-c3ccc(N(c4cc5ccccc5c5ccccc45)c4cc5ccccc5c5ccccc45)cc3)ccc2c1]

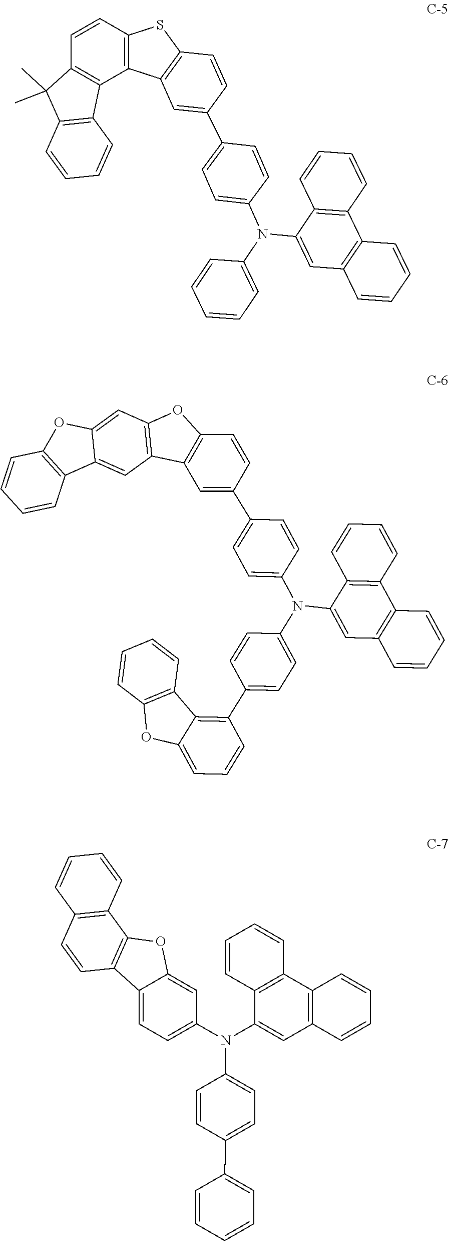 [SMILES: CC1(C)c2ccccc2-c2c1ccc1sc3ccc(-c4ccc(N(c5ccccc5)c5cc6ccccc6c6ccccc56)cc4)cc3c21 ; c1ccc2c(c1)cc(N(c1ccc(-c3ccc4oc5cc6oc7ccccc7c6cc5c4c3)cc1)c1ccc(-c3cccc4oc5ccccc5c34)cc1)c1ccccc12 ; c1ccc(-c2ccc(N(c3ccc4c(c3)oc3c5ccccc5ccc43)c3cc4ccccc4c4ccccc34)cc2)cc1]

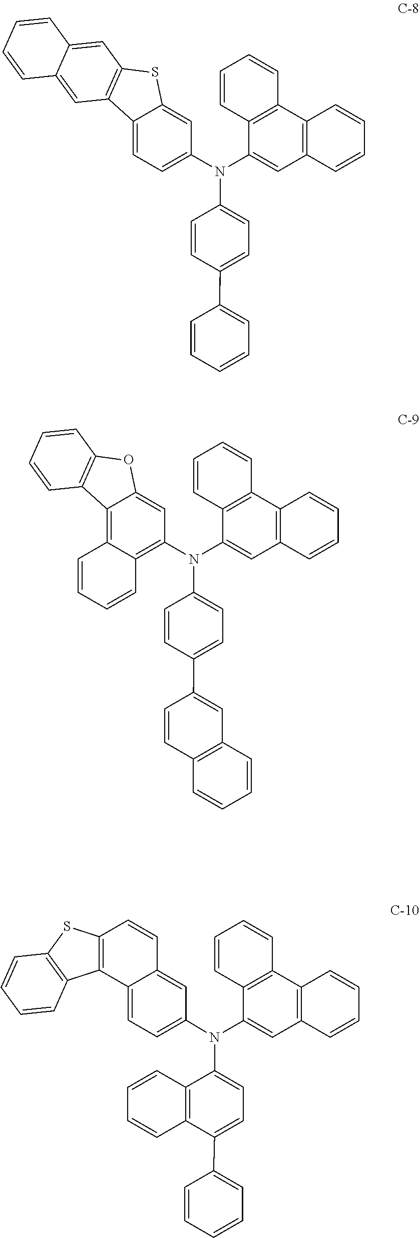 [SMILES: c1ccc(-c2ccc(N(c3ccc4c(c3)sc3cc5ccccc5cc34)c3cc4ccccc4c4ccccc34)cc2)cc1 ; c1ccc2cc(-c3ccc(N(c4cc5ccccc5c5ccccc45)c4cc5oc6ccccc6c5c5ccccc45)cc3)ccc2c1 ; c1ccc(-c2ccc(N(c3ccc4c(ccc5sc6ccccc6c54)c3)c3cc4ccccc4c4ccccc34)c3ccccc23)cc1]

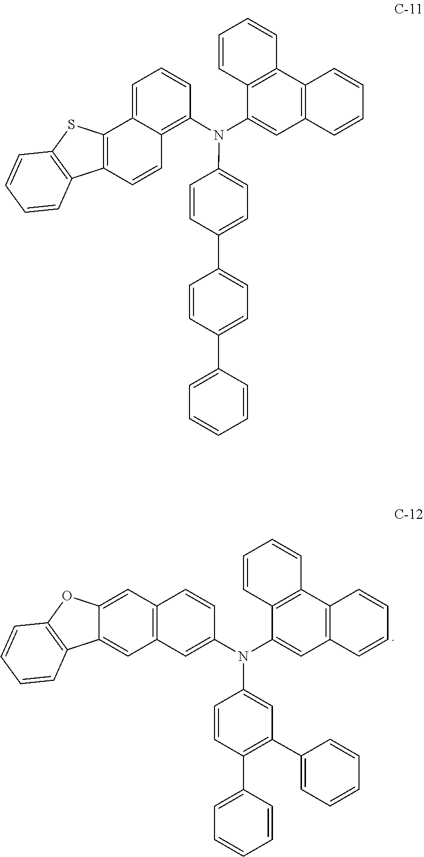 [SMILES: c1ccc(-c2ccc(-c3ccc(N(c4cc5ccccc5c5ccccc45)c4cccc5c4ccc4c6ccccc6sc54)cc3)cc2)cc1 ; c1ccc(-c2ccc(N(c3ccc4cc5oc6ccccc6c5cc4c3)c3cc4ccccc4c4ccccc34)cc2-c2ccccc2)cc1]

### Experimental Example

The voltage, efficiency, and lifetime were measured (based on 15 mA/cm²) by applying a current to the organic light emitting devices manufactured in Examples 1 to 155 and Comparative Examples 1 to 227, and the results are shown in Tables 1 to 4 below. Lifetime T95 means the time required for the luminance to be reduced to 95% of the initial luminance (6,000 nit).

TABLE 1

| | | | | | | Light |
|---|---|---|---|---|---|---|
| | | | Driving | Efficiency | Lifetime | emitting |
| Category | First host | Second host | voltage (V) | (cd/A) | T95 (hr) | color |
| Example 1 | Compound 1-2 | Compound 2-1 | 3.96 | 19.6 | 203 | Red |
| Example 2 | Compound 1-2 | Compound 2-27 | 3.94 | 19.3 | 207 | Red |
| Example 3 | Compound 1-2 | Compound 2-39 | 3.97 | 19.8 | 198 | Red |
| Example 4 | Compound 1-2 | Compound 2-54 | 3.88 | 20.4 | 185 | Red |
| Example 5 | Compound 1-2 | Compound 2-60 | 3.93 | 19.1 | 193 | Red |
| Example 6 | Compound 1-3 | Compound 2-3 | 3.92 | 18.9 | 191 | Red |
| Example 7 | Compound 1-3 | Compound 2-10 | 3.88 | 19.2 | 208 | Red |
| Example 8 | Compound 1-3 | Compound 2-68 | 3.90 | 19.6 | 197 | Red |
| Example 9 | Compound 1-3 | Compound 2-44 | 3.89 | 18.7 | 191 | Red |
| Example 10 | Compound 1-3 | Compound 2-49 | 3.92 | 185 | 205 | Red |
| Example 11 | Compound 1-7 | Compound 2-5 | 3.86 | 19.3 | 213 | Red |
| Example 12 | Compound 1-7 | Compound 2-14 | 3.82 | 19.8 | 212 | Red |
| Example 13 | Compound 1-7 | Compound 2-23 | 3.86 | 18.1 | 207 | Red |
| Example 14 | Compound 1-7 | Compound 2-58 | 3.83 | 19.5 | 218 | Red |
| Example 15 | Compound 1-7 | Compound 2-64 | 3.81 | 18.7 | 195 | Red |
| Example 16 | Compound 1-9 | Compound 2-17 | 3.88 | 19.3 | 203 | Red |
| Example 17 | Compound 1-9 | Compound 2-20 | 3.80 | 19.7 | 210 | Red |
| Example 18 | Compound 1-9 | Compound 2-28 | 3.84 | 19.0 | 207 | Red |
| Example 19 | Compound 1-9 | Compound 2-35 | 3.89 | 18.6 | 201 | Red |
| Example 20 | Compound 1-9 | Compound 2-57 | 3.86 | 19.2 | 195 | Red |
| Example 21 | Compound 1-11 | Compound 2-1 | 3.85 | 18.4 | 203 | Red |
| Example 22 | Compound 1-11 | Compound 2-27 | 3.81 | 18.6 | 205 | Red |
| Example 23 | Compound 1-11 | Compound 2-39 | 3.80 | 18.7 | 197 | Red |
| Example 24 | Compound 1-11 | Compound 2-54 | 3.88 | 19.0 | 206 | Red |
| Example 25 | Compound 1-11 | Compound 2-60 | 3.83 | 18.9 | 194 | Red |
| Example 26 | Compound 1-14 | Compound 2-3 | 3.89 | 20.1 | 213 | Red |
| Example 27 | Compound 1-14 | Compound 2-10 | 3.87 | 20.3 | 215 | Red |
| Example 28 | Compound 1-14 | Compound 2-68 | 3.88 | 20.2 | 201 | Red |
| Example 29 | Compound 1-14 | Compound 2-44 | 3.86 | 19.5 | 217 | Red |
| Example 30 | Compound 1-14 | Compound 2-49 | 3.86 | 20.4 | 220 | Red |
| Example 31 | Compound 1-15 | Compound 2-5 | 3.79 | 19.3 | 208 | Red |
| Example 32 | Compound 1-15 | Compound 2-14 | 3.72 | 20.2 | 211 | Red |
| Example 33 | Compound 1-15 | Compound 2-23 | 3.74 | 20.7 | 210 | Red |
| Example 34 | Compound 1-15 | Compound 2-58 | 3.70 | 19.8 | 203 | Red |
| Example 35 | Compound 1-15 | Compound 2-64 | 3.77 | 20.6 | 215 | Red |
| Example 36 | Compound 1-16 | Compound 2-17 | 3.76 | 19.9 | 221 | Red |
| Example 37 | Compound 1-16 | Compound 2-20 | 3.78 | 20.3 | 216 | Red |
| Example 38 | Compound 1-16 | Compound 2-28 | 3.74 | 20.1 | 220 | Red |
| Example 39 | Compound 1-16 | Compound 2-35 | 3.79 | 20.6 | 208 | Red |
| Example 40 | Compound 1-16 | Compound 2-70 | 3.82 | 20.4 | 215 | Red |
| Example 41 | Compound 1-17 | Compound 2-1 | 3.81 | 20.1 | 203 | Red |
| Example 42 | Compound 1-17 | Compound 2-27 | 3.83 | 20.3 | 214 | Red |
| Example 43 | Compound 1-17 | Compound 2-39 | 3.80 | 20.8 | 209 | Red |
| Example 44 | Compound 1-17 | Compound 2-54 | 3.85 | 20.5 | 205 | Red |
| Example 45 | Compound 1-17 | Compound 2-60 | 3.84 | 21.0 | 215 | Red |
| Example 46 | Compound 1-20 | Compound 2-3 | 3.93 | 19.3 | 201 | Red |
| Example 47 | Compound 1-20 | Compound 2-10 | 3.90 | 20.0 | 228 | Red |
| Example 48 | Compound 1-20 | Compound 2-68 | 3.92 | 20.6 | 210 | Red |
| Example 49 | Compound 1-20 | Compound 2-44 | 3.88 | 19.7 | 224 | Red |
| Example 50 | Compound 1-20 | Compound 2-49 | 3.86 | 20.0 | 217 | Red |
| Example 51 | Compound 1-22 | Compound 2-5 | 3.80 | 20.9 | 234 | Red |
| Example 52 | Compound 1-22 | Compound 2-14 | 3.81 | 20.5 | 221 | Red |
| Example 53 | Compound 1-22 | Compound 2-23 | 3.87 | 19.9 | 216 | Red |
| Example 54 | Compound 1-22 | Compound 2-58 | 3.86 | 20.6 | 224 | Red |
| Example 55 | Compound 1-22 | Compound 2-64 | 3.93 | 20.4 | 225 | Red |
| Example 56 | Compound 1-24 | Compound 2-17 | 3.78 | 20.3 | 217 | Red |
| Example 57 | Compound 1-24 | Compound 2-20 | 3.84 | 21.1 | 230 | Red |
| Example 58 | Compound 1-24 | Compound 2-28 | 3.80 | 21.4 | 228 | Red |
| Example 59 | Compound 1-24 | Compound 2-35 | 3.83 | 20.7 | 233 | Red |
| Example 60 | Compound 1-24 | Compound 2-57 | 3.85 | 20.9 | 215 | Red |
| Example 61 | Compound 1-27 | Compound 2-1 | 3.76 | 20.5 | 238 | Red |
| Example 62 | Compound 1-27 | Compound 2-27 | 3.82 | 19.3 | 211 | Red |
| Example 63 | Compound 1-27 | Compound 2-39 | 3.79 | 19.6 | 223 | Red |
| Example 64 | Compound 1-27 | Compound 2-54 | 3.78 | 19.1 | 226 | Red |
| Example 65 | Compound 1-27 | Compound 2-60 | 3.82 | 20.3 | 230 | Red |
| Example 66 | Compound 1-28 | Compound 2-3 | 3.88 | 19.7 | 227 | Red |
| Example 67 | Compound 1-28 | Compound 2-10 | 3.84 | 20.8 | 218 | Red |
| Example 68 | Compound 1-28 | Compound 2-68 | 3.78 | 19.3 | 215 | Red |
| Example 69 | Compound 1-28 | Compound 2-44 | 3.82 | 19.5 | 220 | Red |
| Example 70 | Compound 1-28 | Compound 2-49 | 3.86 | 20.2 | 218 | Red |
| Example 71 | Compound 1-31 | Compound 2-5 | 3.98 | 20.3 | 256 | Red |
| Example 72 | Compound 1-31 | Compound 2-14 | 3.92 | 20.0 | 248 | Red |
| Example 73 | Compound 1-31 | Compound 2-23 | 3.95 | 20.5 | 251 | Red |
| Example 74 | Compound 1-31 | Compound 2-58 | 3.90 | 20.1 | 252 | Red |
| Example 75 | Compound 1-31 | Compound 2-64 | 3.93 | 19.8 | 265 | Red |
| Example 76 | Compound 1-33 | Compound 2-1 | 3.68 | 22.3 | 281 | Red |
| Example 77 | Compound 1-33 | Compound 2-27 | 3.65 | 22.0 | 277 | Red |
| Example 78 | Compound 1-33 | Compound 2-39 | 3.70 | 21.3 | 288 | Red |
| Example 79 | Compound 1-33 | Compound 2-54 | 3.69 | 22.7 | 270 | Red |
| Example 80 | Compound 1-33 | Compound 2-60 | 3.71 | 21.8 | 274 | Red |
| Example 81 | Compound 1-37 | Compound 2-17 | 3.76 | 22.2 | 263 | Red |
| Example 82 | Compound 1-37 | Compound 2-20 | 3.70 | 21.9 | 275 | Red |
| Example 83 | Compound 1-37 | Compound 2-28 | 3.74 | 22.0 | 279 | Red |
| Example 84 | Compound 1-37 | Compound 2-35 | 3.79 | 21.8 | 256 | Red |
| Example 85 | Compound 1-37 | Compound 2-70 | 3.74 | 21.5 | 275 | Red |
| Example 86 | Compound 1-38 | Compound 2-3 | 3.72 | 21.0 | 275 | Red |
| Example 87 | Compound 1-38 | Compound 2-10 | 3.70 | 22.1 | 271 | Red |
| Example 88 | Compound 1-38 | Compound 2-68 | 3.75 | 21.5 | 284 | Red |
| Example 89 | Compound 1-38 | Compound 2-44 | 3.74 | 21.3 | 268 | Red |
| Example 90 | Compound 1-38 | Compound 2-49 | 3.71 | 22.3 | 275 | Red |
| Example 91 | Compound 1-40 | Compound 2-5 | 3.73 | 20.7 | 225 | Red |
| Example 92 | Compound 1-40 | Compound 2-14 | 3.70 | 19.1 | 238 | Red |
| Example 93 | Compound 1-40 | Compound 2-23 | 3.76 | 19.7 | 231 | Red |
| Example 94 | Compound 1-40 | Compound 2-58 | 3.74 | 19.0 | 240 | Red |
| Example 95 | Compound 1-40 | Compound 2-64 | 3.80 | 19.4 | 234 | Red |
| Example 96 | Compound 1-41 | Compound 2-17 | 3.83 | 19.0 | 241 | Red |
| Example 97 | Compound 1-41 | Compound 2-20 | 3.87 | 19.5 | 236 | Red |
| Example 98 | Compound 1-41 | Compound 2-28 | 3.83 | 18.9 | 228 | Red |
| Example 99 | Compound 1-41 | Compound 2-35 | 3.86 | 19.8 | 234 | Red |
| Example 100 | Compound 1-41 | Compound 2-57 | 3.79 | 19.4 | 225 | Red |
| Example 101 | Compound 1-43 | Compound 2-1 | 3.81 | 19.5 | 197 | Red |
| Example 102 | Compound 1-43 | Compound 2-27 | 3.80 | 20.0 | 202 | Red |
| Example 103 | Compound 1-43 | Compound 2-39 | 3.84 | 19.4 | 213 | Red |
| Example 104 | Compound 1-43 | Compound 2-54 | 3.79 | 20.8 | 204 | Red |
| Example 105 | Compound 1-43 | Compound 2-60 | 3.85 | 19.7 | 195 | Red |
| Example 106 | Compound 1-45 | Compound 2-3 | 3.87 | 20.1 | 194 | Red |
| Example 107 | Compound 1-45 | Compound 2-10 | 3.91 | 18.8 | 203 | Red |
| Example 108 | Compound 1-45 | Compound 2-68 | 3.88 | 19.3 | 201 | Red |
| Example 109 | Compound 1-45 | Compound 2-44 | 3.93 | 20.7 | 198 | Red |
| Example 110 | Compound 1-45 | Compound 2-49 | 3.85 | 18.9 | 194 | Red |
| Example 111 | Compound 1-47 | Compound 2-5 | 3.79 | 20.1 | 209 | Red |
| Example 112 | Compound 1-47 | Compound 2-14 | 3.82 | 20.3 | 201 | Red |
| Example 113 | Compound 1-47 | Compound 2-23 | 3.84 | 20.0 | 197 | Red |
| Example 114 | Compound 1-47 | Compound 2-58 | 3.86 | 19.9 | 194 | Red |
| Example 115 | Compound 1-47 | Compound 2-64 | 3.80 | 19.6 | 205 | Red |
| Example 116 | Compound 1-48 | Compound 2-17 | 3.83 | 19.6 | 217 | Red |
| Example 117 | Compound 1-48 | Compound 2-20 | 3.87 | 18.8 | 214 | Red |
| Example 118 | Compound 1-48 | Compound 2-28 | 3.80 | 19.7 | 203 | Red |
| Example 119 | Compound 1-48 | Compound 2-35 | 3.88 | 19.4 | 198 | Red |
| Example 120 | Compound 1-48 | Compound 2-70 | 3.92 | 18.9 | 201 | Red |
| Example 121 | Compound 1-52 | Compound 2-1 | 3.91 | 19.6 | 197 | Red |
| Example 122 | Compound 1-52 | Compound 2-27 | 3.94 | 19.3 | 190 | Red |
| Example 123 | Compound 1-52 | Compound 2-39 | 3.92 | 19.2 | 203 | Red |
| Example 124 | Compound 1-52 | Compound 2-54 | 3.88 | 18.9 | 194 | Red |
| Example 125 | Compound 1-52 | Compound 2-60 | 3.94 | 19.7 | 191 | Red |
| Example 126 | Compound 1-53 | Compound 2-3 | 3.87 | 18.8 | 203 | Red |
| Example 127 | Compound 1-53 | Compound 2-10 | 3.90 | 19.1 | 209 | Red |
| Example 128 | Compound 1-53 | Compound 2-68 | 3.86 | 18.6 | 198 | Red |
| Example 129 | Compound 1-53 | Compound 2-44 | 3.93 | 19.0 | 204 | Red |
| Example 130 | Compound 1-53 | Compound 2-49 | 3.90 | 19.3 | 195 | Red |
| Example 131 | Compound 1-55 | Compound 2-5 | 3.87 | 20.3 | 223 | Red |
| Example 132 | Compound 1-55 | Compound 2-14 | 3.82 | 19.9 | 224 | Red |
| Example 133 | Compound 1-55 | Compound 2-23 | 3.83 | 20.6 | 218 | Red |
| Example 134 | Compound 1-55 | Compound 2-58 | 3.84 | 20.4 | 208 | Red |
| Example 135 | Compound 1-55 | Compound 2-64 | 3.90 | 19.7 | 225 | Red |
| Example 136 | Compound 1-56 | Compound 2-17 | 3.98 | 20.0 | 195 | Red |
| Example 137 | Compound 1-56 | Compound 2-20 | 3.94 | 20.7 | 192 | Red |
| Example 138 | Compound 1-56 | Compound 2-28 | 3.92 | 19.4 | 197 | Red |
| Example 139 | Compound 1-56 | Compound 2-35 | 3.95 | 20.3 | 203 | Red |
| Example 140 | Compound 1-56 | Compound 2-57 | 3.97 | 19.8 | 195 | Red |
| Example 141 | Compound 1-57 | Compound 2-1 | 3.91 | 18.1 | 213 | Red |
| Example 142 | Compound 1-57 | Compound 2-27 | 3.94 | 19.0 | 220 | Red |
| Example 143 | Compound 1-57 | Compound 2-39 | 3.88 | 18.5 | 229 | Red |
| Example 144 | Compound 1-57 | Compound 2-54 | 3.93 | 18.9 | 217 | Red |
| Example 145 | Compound 1-57 | Compound 2-60 | 3.90 | 19.4 | 224 | Red |
| Example 146 | Compound 1-58 | Compound 2-3 | 3.87 | 18.7 | 199 | Red |
| Example 147 | Compound 1-58 | Compound 2-10 | 3.90 | 18.5 | 201 | Red |
| Example 148 | Compound 1-58 | Compound 2-68 | 3.93 | 19.2 | 203 | Red |
| Example 149 | Compound 1-58 | Compound 2-44 | 3.87 | 19.4 | 195 | Red |
| Example 150 | Compound 1-58 | Compound 2-49 | 3.84 | 19.0 | 198 | Red |
| Example 151 | Compound 1-60 | Compound 2-5 | 3.94 | 18.9 | 203 | Red |
| Example 152 | Compound 1-60 | Compound 2-14 | 3.85 | 19.1 | 197 | Red |
| Example 153 | Compound 1-60 | Compound 2-23 | 3.88 | 18.5 | 211 | Red |
| Example 154 | Compound 1-60 | Compound 2-58 | 3.95 | 19.6 | 207 | Red |
| Example 155 | Compound 1-60 | Compound 2-64 | 3.91 | 19.4 | 208 | Red |

TABLE 2

| | | Electron | | | | Light |
|---|---|---|---|---|---|---|
| | | blocking | Driving | Efficiency | Lifetime | emitting |
| Category | Host | layer | voltage(V) | (cd/A) | T95(hr) | color |
| Comparative | Compound 1-2 | Compound EB-1 | 4.37 | 14.8 | 117 | Red |
| Example 1 | | | | | | |
| Comparative | Compound 1-2 | Compound 2-1 | 4.39 | 14.0 | 101 | Red |
| Example 2 | | | | | | |
| Comparative | Compound 1-2 | Compound 2-27 | 4.48 | 14.1 | 103 | Red |
| Example 3 | | | | | | |
| Comparative | Compound 1-2 | Compound 2-39 | 4.49 | 14.8 | 98 | Red |
| Example 4 | | | | | | |
| Comparative | Compound 1-2 | Compound 2-54 | 4.40 | 15.5 | 92 | Red |
| Example 5 | | | | | | |
| Comparative | Compound 1-2 | Compound 2-60 | 4.43 | 15.1 | 111 | Red |
| Example 6 | | | | | | |
| Comparative | Compound 1-11 | Compound 2-3 | 4.36 | 15.2 | 114 | Red |
| Example 7 | | | | | | |
| Comparative | Compound 1-11 | Compound 2-10 | 4.39 | 15.0 | 103 | Red |
| Example 8 | | | | | | |
| Comparative | Compound 1-11 | Compound 2-68 | 4.41 | 15.3 | 104 | Red |
| Example 9 | | | | | | |
| Comparative | Compound 1-11 | Compound 2-44 | 4.42 | 14.4 | 107 | Red |
| Example 10 | | | | | | |
| Comparative | Compound 1-11 | Compound 2-49 | 4.39 | 14.9 | 106 | Red |
| Example 11 | | | | | | |
| Comparative | Compound 1-15 | Compound 2-5 | 4.40 | 15.2 | 138 | Red |
| Example 12 | | | | | | |
| Comparative | Compound 1-15 | Compound 2-14 | 4.45 | 15.6 | 127 | Red |
| Example 13 | | | | | | |
| Comparative | Compound 1-15 | Compound 2-23 | 4.43 | 15.0 | 121 | Red |
| Example 14 | | | | | | |
| Comparative | Compound 1-15 | Compound 2-58 | 4.48 | 15.3 | 114 | Red |
| Example 15 | | | | | | |
| Comparative | Compound 1-15 | Compound 2-64 | 4.45 | 15.7 | 121 | Red |
| Example 16 | | | | | | |
| Comparative | Compound 1-17 | Compound 2-17 | 4.43 | 15.3 | 105 | Red |
| Example 17 | | | | | | |
| Comparative | Compound 1-17 | Compound 2-20 | 4.44 | 15.4 | 103 | Red |
| Example 18 | | | | | | |
| Comparative | Compound 1-17 | Compound 2-28 | 4.46 | 15.4 | 110 | Red |
| Example 19 | | | | | | |
| Comparative | Compound 1-17 | Compound 2-35 | 4.41 | 14.8 | 107 | Red |
| Example 20 | | | | | | |
| Comparative | Compound 1-17 | Compound 2-70 | 4.44 | 14.9 | 111 | Red |
| Example 21 | | | | | | |
| Comparative | Compound 1-27 | Compound 2-1 | 4.38 | 15.0 | 108 | Red |
| Example 22 | | | | | | |
| Comparative | Compound 1-27 | Compound 2-27 | 4.37 | 14.8 | 103 | Red |
| Example 23 | | | | | | |
| Comparative | Compound 1-27 | Compound 2-39 | 4.36 | 14.1 | 100 | Red |
| Example 24 | | | | | | |
| Comparative | Compound 1-27 | Compound 2-54 | 4.39 | 14.4 | 101 | Red |
| Example 25 | | | | | | |
| Comparative | Compound 1-27 | Compound 2-60 | 4.38 | 14.7 | 111 | Red |
| Example 26 | | | | | | |
| Comparative | Compound 1-37 | Compound 2-3 | 4.37 | 15.3 | 106 | Red |
| Example 27 | | | | | | |
| Comparative | Compound 1-37 | Compound 2-10 | 4.39 | 15.4 | 108 | Red |
| Example 28 | | | | | | |
| Comparative | Compound 1-37 | Compound 2-68 | 4.38 | 15.3 | 117 | Red |
| Example 29 | | | | | | |
| Comparative | Compound 1-37 | Compound 2-44 | 4.37 | 15.0 | 104 | Red |
| Example 30 | | | | | | |
| Comparative | Compound 1-37 | Compound 2-49 | 4.36 | 15.1 | 111 | Red |
| Example 31 | | | | | | |
| Comparative | Compound 1-41 | Compound 2-5 | 4.42 | 14.6 | 109 | Red |
| Example 32 | | | | | | |
| Comparative | Compound 1-41 | Compound 2-14 | 4.45 | 14.0 | 91 | Red |
| Example 33 | | | | | | |
| Comparative | Compound 1-41 | Compound 2-23 | 4.36 | 14.2 | 95 | Red |
| Example 34 | | | | | | |
| Comparative | Compound 1-41 | Compound 2-58 | 4.39 | 14.3 | 108 | Red |
| Example 35 | | | | | | |
| Comparative | Compound 1-41 | Compound 2-64 | 4.35 | 14.0 | 94 | Red |
| Example 36 | | | | | | |
| Comparative | Compound 1-45 | Compound 2-17 | 4.38 | 14.1 | 92 | Red |
| Example 37 | | | | | | |
| Comparative | Compound 1-45 | Compound 2-20 | 4.39 | 14.5 | 98 | Red |
| Example 38 | | | | | | |
| Comparative | Compound 1-45 | Compound 2-28 | 4.37 | 14.3 | 95 | Red |
| Example 39 | | | | | | |
| Comparative | Compound 1-45 | Compound 2-35 | 4.36 | 14.7 | 91 | Red |
| Example 40 | | | | | | |
| Comparative | Compound 1-45 | Compound 2-57 | 4.38 | 14.0 | 89 | Red |
| Example 41 | | | | | | |
| Comparative | Compound 1-53 | Compound 2-1 | 4.38 | 15.1 | 97 | Red |
| Example 42 | | | | | | |
| Comparative | Compound 1-53 | Compound 2-7 | 4.39 | 15.3 | 101 | Red |
| Example 43 | | | | | | |
| Comparative | Compound 1-53 | Compound 2-39 | 4.42 | 15.0 | 96 | Red |
| Example 44 | | | | | | |
| Comparative | Compound 1-53 | Compound 2-54 | 4.40 | 14.8 | 98 | Red |
| Example 45 | | | | | | |
| Comparative | Compound 1-53 | Compound 2-60 | 4.34 | 14.9 | 94 | Red |
| Example 46 | | | | | | |
| Comparative | Compound 1-55 | Compound 2-3 | 4.46 | 14.7 | 104 | Red |
| Example 47 | | | | | | |
| Comparative | Compound 1-55 | Compound 2-10 | 4.38 | 15.0 | 107 | Red |
| Example 48 | | | | | | |
| Comparative | Compound 1-55 | Compound 2-68 | 4.39 | 14.8 | 106 | Red |
| Example 49 | | | | | | |
| Comparative | Compound 1-55 | Compound 2-44 | 4.47 | 15.3 | 104 | Red |
| Example 50 | | | | | | |
| Comparative | Compound 1-55 | Compound 2-49 | 4.42 | 15.0 | 112 | Red |
| Example 51 | | | | | | |
| Comparative | Compound 1-57 | Compound 2-5 | 4.41 | 14.1 | 101 | Red |
| Example 52 | | | | | | |
| Comparative | Compound 1-57 | Compound 2-14 | 4.40 | 14.9 | 107 | Red |
| Example 53 | | | | | | |
| Comparative | Compound 1-57 | Compound 2-23 | 4.43 | 14.6 | 108 | Red |
| Example 54 | | | | | | |
| Comparative | Compound 1-57 | Compound 2-58 | 4.39 | 14.7 | 99 | Red |
| Example 55 | | | | | | |
| Comparative | Compound 1-57 | Compound 2-64 | 4.45 | 15.0 | 101 | Red |
| Example 56 | | | | | | |
| Comparative | Compound 1-60 | Compound 2-17 | 4.40 | 14.5 | 91 | Red |
| Example 57 | | | | | | |
| Comparative | Compound 1-60 | Compound 2-20 | 4.41 | 15.0 | 99 | Red |
| Example 58 | | | | | | |
| Comparative | Compound 1-60 | Compound 2-28 | 4.42 | 14.3 | 104 | Red |
| Example 59 | | | | | | |
| Comparative | Compound 1-60 | Compound 2-35 | 4.44 | 15.1 | 96 | Red |
| Example 60 | | | | | | |
| Comparative | Compound 1-60 | Compound 2-70 | 4.39 | 14.5 | 93 | Red |
| Example 61 | | | | | | |

TABLE 3

| | | | | | | Light |
|---|---|---|---|---|---|---|
| | | | Driving | Efficiency | Lifetime | emitting |
| Category | First host | Second host | voltage(V) | (cd/A) | T95(hr) | color |
| Comparative | Compound B-1 | Compound 2-1 | 4.31 | 13.1 | 91 | Red |
| Example 62 | | | | | | |
| Comparative | Compound B-1 | Compound 2-27 | 4.36 | 13.8 | 103 | Red |
| Example 63 | | | | | | |
| Comparative | Compound B-1 | Compound 2-39 | 4.34 | 13.6 | 97 | Red |
| Example 64 | | | | | | |
| Comparative | Compound B-1 | Compound 2-54 | 4.39 | 14.0 | 102 | Red |
| Example 65 | | | | | | |
| Comparative | Compound B-1 | Compound 2-60 | 4.36 | 13.9 | 95 | Red |
| Example 66 | | | | | | |
| Comparative | Compound B-2 | Compound 2-3 | 4.33 | 13.3 | 84 | Red |
| Example 67 | | | | | | |
| Comparative | Compound B-2 | Compound 2-10 | 4.40 | 13.8 | 93 | Red |
| Example 68 | | | | | | |
| Comparative | Compound B-2 | Compound 2-68 | 4.38 | 13.6 | 95 | Red |
| Example 69 | | | | | | |
| Comparative | Compound B-2 | Compound 2-44 | 4.31 | 13.9 | 87 | Red |
| Example 70 | | | | | | |
| Comparative | Compound B-2 | Compound 2-49 | 4.36 | 14.1 | 95 | Red |
| Example 71 | | | | | | |
| Comparative | Compound B-3 | Compound 2-5 | 4.40 | 13.2 | 108 | Red |
| Example 72 | | | | | | |
| Comparative | Compound B-3 | Compound 2-14 | 4.43 | 14.0 | 97 | Red |
| Example 73 | | | | | | |
| Comparative | Compound B-3 | Compound 2-23 | 4.41 | 13.4 | 101 | Red |
| Example 74 | | | | | | |
| Comparative | Compound B-3 | Compound 2-58 | 4.39 | 14.3 | 104 | Red |
| Example 75 | | | | | | |
| Comparative | Compound B-3 | Compound 2-64 | 4.42 | 14.2 | 101 | Red |
| Example 76 | | | | | | |
| Comparative | Compound B-4 | Compound 2-17 | 4.35 | 13.8 | 90 | Red |
| Example 77 | | | | | | |
| Comparative | Compound B-4 | Compound 2-20 | 4.38 | 14.2 | 97 | Red |
| Example 78 | | | | | | |
| Comparative | Compound B-4 | Compound 2-28 | 4.36 | 13.6 | 91 | Red |
| Example 79 | | | | | | |
| Comparative | Compound B-4 | Compound 2-35 | 4.40 | 14.1 | 92 | Red |
| Example 80 | | | | | | |
| Comparative | Compound B-4 | Compound 2-57 | 4.42 | 13.7 | 89 | Red |
| Example 81 | | | | | | |
| Comparative | Compound B-5 | Compound 2-1 | 4.27 | 15.0 | 128 | Red |
| Example 82 | | | | | | |
| Comparative | Compound B-5 | Compound 2-27 | 4.29 | 14.8 | 127 | Red |
| Example 83 | | | | | | |
| Comparative | Compound B-5 | Compound 2-39 | 4.30 | 15.3 | 124 | Red |
| Example 84 | | | | | | |
| Comparative | Compound B-5 | Compound 2-54 | 4.27 | 14.9 | 121 | Red |
| Example 85 | | | | | | |
| Comparative | Compound B-5 | Compound 2-60 | 4.29 | 14.7 | 128 | Red |
| Example 86 | | | | | | |
| Comparative | Compound B-6 | Compound 2-3 | 4.30 | 15.3 | 114 | Red |
| Example 87 | | | | | | |
| Comparative | Compound B-6 | Compound 2-10 | 4.28 | 15.0 | 124 | Red |
| Example 88 | | | | | | |
| Comparative | Compound B-6 | Compound 2-68 | 4.26 | 15.5 | 117 | Red |
| Example 89 | | | | | | |
| Comparative | Compound B-6 | Compound 2-44 | 4.31 | 15.8 | 124 | Red |
| Example 90 | | | | | | |
| Comparative | Compound B-6 | Compound 2-49 | 4.27 | 14.6 | 111 | Red |
| Example 91 | | | | | | |
| Comparative | Compound B-7 | Compound 2-5 | 4.29 | 15.6 | 129 | Red |
| Example 92 | | | | | | |
| Comparative | Compound B-7 | Compound 2-14 | 4.31 | 14.7 | 131 | Red |
| Example 93 | | | | | | |
| Comparative | Compound B-7 | Compound 2-23 | 4.27 | 15.0 | 125 | Red |
| Example 94 | | | | | | |
| Comparative | Compound B-7 | Compound 2-58 | 4.24 | 14.9 | 117 | Red |
| Example 95 | | | | | | |
| Comparative | Compound B-7 | Compound 2-64 | 4.28 | 14.6 | 124 | Red |
| Example 96 | | | | | | |
| Comparative | Compound B-8 | Compound 2-17 | 4.21 | 14.0 | 112 | Red |
| Example 97 | | | | | | |
| Comparative | Compound B-8 | Compound 2-20 | 4.29 | 15.3 | 116 | Red |
| Example 98 | | | | | | |
| Comparative | Compound B-8 | Compound 2-28 | 4.25 | 14.1 | 120 | Red |
| Example 99 | | | | | | |
| Comparative | Compound B-8 | Compound 2-35 | 4.23 | 15.2 | 109 | Red |
| Example 100 | | | | | | |
| Comparative | Compound B-8 | Compound 2-57 | 4.20 | 15.0 | 124 | Red |
| Example 101 | | | | | | |
| Comparative | Compound B-9 | Compound 2-1 | 4.11 | 15.5 | 143 | Red |
| Example 102 | | | | | | |
| Comparative | Compound B-9 | Compound 2-27 | 4.10 | 15.7 | 139 | Red |
| Example 103 | | | | | | |
| Comparative | Compound B-9 | Compound 2-39 | 4.13 | 15.8 | 137 | Red |
| Example 104 | | | | | | |
| Comparative | Compound B-9 | Compound 2-54 | 4.15 | 14.9 | 130 | Red |
| Example 105 | | | | | | |
| Comparative | Compound B-9 | Compound 2-60 | 4.09 | 15.1 | 141 | Red |
| Example 106 | | | | | | |
| Comparative | Compound B-10 | Compound 2-3 | 4.13 | 15.6 | 137 | Red |
| Example 107 | | | | | | |
| Comparative | Compound B-10 | Compound 2-10 | 4.15 | 16.1 | 126 | Red |
| Example 108 | | | | | | |
| Comparative | Compound B-10 | Compound 2-68 | 4.17 | 16.0 | 134 | Red |
| Example 109 | | | | | | |
| Comparative | Compound B-10 | Compound 2-44 | 4.10 | 15.7 | 126 | Red |
| Example 110 | | | | | | |
| Comparative | Compound B-10 | Compound 2-49 | 4.14 | 15.4 | 129 | Red |
| Example 111 | | | | | | |
| Comparative | Compound B-11 | Compound 2-5 | 4.10 | 15.8 | 142 | Red |
| Example 112 | | | | | | |
| Comparative | Compound B-11 | Compound 2-14 | 4.09 | 16.3 | 137 | Red |
| Example 113 | | | | | | |
| Comparative | Compound B-11 | Compound 2-23 | 4.17 | 15.9 | 130 | Red |
| Example 114 | | | | | | |
| Comparative | Compound B-11 | Compound 2-58 | 4.16 | 16.4 | 124 | Red |
| Example 115 | | | | | | |
| Comparative | Compound B-11 | Compound 2-64 | 4.18 | 16.1 | 132 | Red |
| Example 116 | | | | | | |
| Comparative | Compound B-12 | Compound 2-17 | 4.09 | 16.0 | 147 | Red |
| Example 117 | | | | | | |
| Comparative | Compound B-12 | Compound 2-20 | 4.12 | 16.5 | 129 | Red |
| Example 118 | | | | | | |
| Comparative | Compound B-12 | Compound 2-28 | 4.08 | 16.3 | 144 | Red |
| Example 119 | | | | | | |
| Comparative | Compound B-12 | Compound 2-35 | 4.17 | 15.7 | 136 | Red |
| Example 120 | | | | | | |
| Comparative | Compound B-12 | Compound 2-70 | 4.13 | 15.4 | 141 | Red |
| Example 121 | | | | | | |

TABLE 4

| | | | | | | Light |
|---|---|---|---|---|---|---|
| | | | Driving | Efficiency | Lifetime | emitting |
| Category | First host | Second host | voltage(V) | (cd/A) | T95 (hr) | color |
| Comparative | Compound 1-2 | Compound C-1 | 4.21 | 15.0 | 131 | Red |
| Example 122 | | | | | | |
| Comparative | Compound 1-11 | Compound C-1 | 4.26 | 14.8 | 123 | Red |
| Example 123 | | | | | | |
| Comparative | Compound 1-15 | Compound C-1 | 4.17 | 15.3 | 117 | Red |
| Example 124 | | | | | | |
| Comparative | Compound 1-28 | Compound C-1 | 4.25 | 14.9 | 128 | Red |
| Example 125 | | | | | | |
| Comparative | Compound 1-33 | Compound C-1 | 4.16 | 14.7 | 105 | Red |
| Example 126 | | | | | | |
| Comparative | Compound 1-40 | Compound C-1 | 4.23 | 15.3 | 122 | Red |
| Example 127 | | | | | | |
| Comparative | Compound 1-43 | Compound C-1 | 4.30 | 15.8 | 111 | Red |
| Example 128 | | | | | | |
| Comparative | Compound 1-55 | Compound C-1 | 4.28 | 14.6 | 130 | Red |
| Example 129 | | | | | | |
| Comparative | Compound 1-3 | Compound C-2 | 4.20 | 14.0 | 114 | Red |
| Example 130 | | | | | | |
| Comparative | Compound 1-7 | Compound C-2 | 4.16 | 14.8 | 105 | Red |
| Example 131 | | | | | | |
| Comparative | Compound 1-17 | Compound C-2 | 4.30 | 15.3 | 109 | Red |
| Example 132 | | | | | | |
| Comparative | Compound 1-24 | Compound C-2 | 4.13 | 14.9 | 102 | Red |
| Example 133 | | | | | | |
| Comparative | Compound 1-37 | Compound C-2 | 4.21 | 15.7 | 123 | Red |
| Example 134 | | | | | | |
| Comparative | Compound 1-47 | Compound C-2 | 4.19 | 16.0 | 127 | Red |
| Example 135 | | | | | | |
| Comparative | Compound 1-48 | Compound C-2 | 4.22 | 15.4 | 120 | Red |
| Example 136 | | | | | | |
| Comparative | Compound 1-58 | Compound C-2 | 4.31 | 15.1 | 114 | Red |
| Example 137 | | | | | | |
| Comparative | Compound 1-9 | Compound C-3 | 4.18 | 14.3 | 135 | Red |
| Example 138 | | | | | | |
| Comparative | Compound 1-16 | Compound C-3 | 4.26 | 15.1 | 134 | Red |
| Example 139 | | | | | | |
| Comparative | Compound 1-22 | Compound C-3 | 4.20 | 14.7 | 129 | Red |
| Example 140 | | | | | | |
| Comparative | Compound 1-38 | Compound C-3 | 4.12 | 14.0 | 135 | Red |
| Example 141 | | | | | | |
| Comparative | Compound 1-41 | Compound C-3 | 4.17 | 14.9 | 124 | Red |
| Example 142 | | | | | | |
| Comparative | Compound 1-45 | Compound C-3 | 4.24 | 15.1 | 132 | Red |
| Example 143 | | | | | | |
| Comparative | Compound 1-53 | Compound C-3 | 4.22 | 14.8 | 127 | Red |
| Example 144 | | | | | | |
| Comparative | Compound 1-57 | Compound C-3 | 4.22 | 15.6 | 121 | Red |
| Example 145 | | | | | | |
| Comparative | Compound 1-2 | Compound C-4 | 4.19 | 17.0 | 141 | Red |
| Example 146 | | | | | | |
| Comparative | Compound 1-14 | Compound C-4 | 4.20 | 16.8 | 135 | Red |
| Example 147 | | | | | | |
| Comparative | Compound 1-20 | Compound C-4 | 4.21 | 15.7 | 149 | Red |
| Example 148 | | | | | | |
| Comparative | Compound 1-27 | Compound C-4 | 4.16 | 17.9 | 136 | Red |
| Example 149 | | | | | | |
| Comparative | Compound 1-31 | Compound C-4 | 4.11 | 16.3 | 148 | Red |
| Example 150 | | | | | | |
| Comparative | Compound 1-52 | Compound C-4 | 4.17 | 17.3 | 152 | Red |
| Example 151 | | | | | | |
| Comparative | Compound 1-56 | Compound C-4 | 4.19 | 16.8 | 134 | Red |
| Example 152 | | | | | | |
| Comparative | Compound 1-60 | Compound C-4 | 4.15 | 17.6 | 141 | Red |
| Example 153 | | | | | | |
| Comparative | Compound 1-2 | Compound C-5 | 4.37 | 13.0 | 81 | Red |
| Example 154 | | | | | | |
| Comparative | Compound 1-11 | Compound C-5 | 4.34 | 13.9 | 92 | Red |
| Example 155 | | | | | | |
| Comparative | Compound 1-15 | Compound C-5 | 4.38 | 13.1 | 89 | Red |
| Example 156 | | | | | | |
| Comparative | Compound 1-28 | Compound C-5 | 4.41 | 12.8 | 93 | Red |
| Example 157 | | | | | | |
| Comparative | Compound 1-33 | Compound C-5 | 4.39 | 13.3 | 95 | Red |
| Example 158 | | | | | | |
| Comparative | Compound 1-40 | Compound C-5 | 4.31 | 13.8 | 87 | Red |
| Example 159 | | | | | | |
| Comparative | Compound 1-43 | Compound C-5 | 4.33 | 13.2 | 84 | Red |
| Example 160 | | | | | | |
| Comparative | Compound 1-55 | Compound C-5 | 4.34 | 13.0 | 80 | Red |
| Example 161 | | | | | | |
| Comparative | Compound 1-3 | Compound C-6 | 4.31 | 14.1 | 86 | Red |
| Example 162 | | | | | | |
| Comparative | Compound 1-7 | Compound C-6 | 4.30 | 14.4 | 80 | Red |
| Example 163 | | | | | | |
| Comparative | Compound 1-17 | Compound C-6 | 4.35 | 15.1 | 93 | Red |
| Example 164 | | | | | | |
| Comparative | Compound 1-24 | Compound C-6 | 4.33 | 14.7 | 92 | Red |
| Example 165 | | | | | | |
| Comparative | Compound 1-37 | Compound C-6 | 4.31 | 14.6 | 95 | Red |
| Example 166 | | | | | | |
| Comparative | Compound 1-47 | Compound C-6 | 4.29 | 15.3 | 97 | Red |
| Example 167 | | | | | | |
| Comparative | Compound 1-48 | Compound C-6 | 4.32 | 14.8 | 92 | Red |
| Example 168 | | | | | | |
| Comparative | Compound 1-58 | Compound C-6 | 4.30 | 15.3 | 86 | Red |
| Example 169 | | | | | | |
| Comparative | Compound 1-9 | Compound C-7 | 4.29 | 16.6 | 129 | Red |
| Example 170 | | | | | | |
| Comparative | Compound 1-16 | Compound C-7 | 4.23 | 16.1 | 131 | Red |
| Example 171 | | | | | | |
| Comparative | Compound 1-22 | Compound C-7 | 4.27 | 16.5 | 134 | Red |
| Example 172 | | | | | | |
| Comparative | Compound 1-38 | Compound C-7 | 4.28 | 15.8 | 140 | Red |
| Example 173 | | | | | | |
| Comparative | Compound 1-41 | Compound C-7 | 4.20 | 15.9 | 137 | Red |
| Example 174 | | | | | | |
| Comparative | Compound 1-45 | Compound C-7 | 4.29 | 16.3 | 143 | Red |
| Example 175 | | | | | | |
| Comparative | Compound 1-53 | Compound C-7 | 4.30 | 16.8 | 129 | Red |
| Example 176 | | | | | | |
| Comparative | Compound 1-57 | Compound C-7 | 4.27 | 16.2 | 143 | Red |
| Example 177 | | | | | | |
| Comparative | Compound 1-2 | Compound C-8 | 4.15 | 17.2 | 132 | Red |
| Example 178 | | | | | | |
| Comparative | Compound 1-14 | Compound C-8 | 4.13 | 17.3 | 124 | Red |
| Example 179 | | | | | | |
| Comparative | Compound 1-20 | Compound C-8 | 4.21 | 16.9 | 126 | Red |
| Example 180 | | | | | | |
| Comparative | Compound 1-27 | Compound C-8 | 4.18 | 16.2 | 119 | Red |
| Example 181 | | | | | | |
| Comparative | Compound 1-31 | Compound C-8 | 4.12 | 16.1 | 137 | Red |
| Example 182 | | | | | | |
| Comparative | Compound 1-52 | Compound C-8 | 4.17 | 16.7 | 124 | Red |
| Example 183 | | | | | | |
| Comparative | Compound 1-56 | Compound C-8 | 4.20 | 17.2 | 130 | Red |
| Example 184 | | | | | | |
| Comparative | Compound 1-60 | Compound C-8 | 4.19 | 17.5 | 142 | Red |
| Example 185 | | | | | | |
| Comparative | Compound 1-2 | Compound C-9 | 4.21 | 15.3 | 102 | Red |
| Example 186 | | | | | | |
| Comparative | Compound 1-11 | Compound C-9 | 4.16 | 15.6 | 107 | Red |
| Example 187 | | | | | | |
| Comparative | Compound 1-15 | Compound C-9 | 4.24 | 15.1 | 105 | Red |
| Example 188 | | | | | | |
| Comparative | Compound 1-28 | Compound C-9 | 4.27 | 16.0 | 94 | Red |
| Example 189 | | | | | | |
| Comparative | Compound 1-33 | Compound C-9 | 4.19 | 15.3 | 103 | Red |
| Example 190 | | | | | | |
| Comparative | Compound 1-40 | Compound C-9 | 4.22 | 15.8 | 98 | Red |
| Example 201 | | | | | | |
| Comparative | Compound 1-43 | Compound C-9 | 4.26 | 14.7 | 91 | Red |
| Example 202 | | | | | | |
| Comparative | Compound 1-55 | Compound C-9 | 4.27 | 14.9 | 97 | Red |
| Example 203 | | | | | | |
| Comparative | Compound 1-3 | Compound C-10 | 4.20 | 16.9 | 106 | Red |
| Example 204 | | | | | | |
| Comparative | Compound 1-7 | Compound C-10 | 4.21 | 15.4 | 100 | Red |
| Example 205 | | | | | | |
| Comparative | Compound 1-17 | Compound C-10 | 4.19 | 16.8 | 109 | Red |
| Example 206 | | | | | | |
| Comparative | Compound 1-24 | Compound C-10 | 4.22 | 15.0 | 98 | Red |
| Example 207 | | | | | | |
| Comparative | Compound 1-37 | Compound C-10 | 4.17 | 16.2 | 112 | Red |
| Example 208 | | | | | | |
| Comparative | Compound 1-47 | Compound C-10 | 4.26 | 16.7 | 101 | Red |
| Example 209 | | | | | | |
| Comparative | Compound 1-48 | Compound C-10 | 4.28 | 16.0 | 94 | Red |
| Example 210 | | | | | | |
| Comparative | Compound 1-58 | Compound C-10 | 4.26 | 15.4 | 110 | Red |
| Example 211 | | | | | | |
| Comparative | Compound 1-9 | Compound C-11 | 4.31 | 17.2 | 116 | Red |
| Example 212 | | | | | | |
| Comparative | Compound 1-16 | Compound C-11 | 4.30 | 16.6 | 121 | Red |
| Example 213 | | | | | | |
| Comparative | Compound 1-22 | Compound C-11 | 4.35 | 17.1 | 108 | Red |
| Example 214 | | | | | | |
| Comparative | Compound 1-38 | Compound C-11 | 4.35 | 16.4 | 129 | Red |
| Example 215 | | | | | | |
| Comparative | Compound 1-41 | Compound C-11 | 4.34 | 17.8 | 120 | Red |
| Example 216 | | | | | | |
| Comparative | Compound 1-45 | Compound C-11 | 4.31 | 17.8 | 113 | Red |
| Example 217 | | | | | | |
| Comparative | Compound 1-53 | Compound C-11 | 4.32 | 16.5 | 114 | Red |
| Example 218 | | | | | | |
| Comparative | Compound 1-57 | Compound C-11 | 4.39 | 16.9 | 109 | Red |
| Example 219 | | | | | | |
| Comparative | Compound 1-2 | Compound C-12 | 4.18 | 16.7 | 103 | Red |
| Example 220 | | | | | | |
| Comparative | Compound 1-14 | Compound C-12 | 4.21 | 17.0 | 92 | Red |
| Example 221 | | | | | | |
| Comparative | Compound 1-20 | Compound C-12 | 4.23 | 17.4 | 99 | Red |
| Example 222 | | | | | | |
| Comparative | Compound 1-27 | Compound C-12 | 4.20 | 16.8 | 108 | Red |
| Example 223 | | | | | | |
| Comparative | Compound 1-31 | Compound C-12 | 4.27 | 17.2 | 96 | Red |
| Example 224 | | | | | | |
| Comparative | Compound 1-52 | Compound C-12 | 4.26 | 17.3 | 91 | Red |
| Example 225 | | | | | | |
| Comparative | Compound 1-56 | Compound C-12 | 4.24 | 17.5 | 105 | Red |
| Example 226 | | | | | | |
| Comparative | Compound 1-60 | Compound C-12 | 4.19 | 16.1 | 112 | Red |
| Example 227 | | | | | | |

When a current was applied to the organic light emitting devices manufactured according to Examples 1 to 155 and Comparative Examples 1 to 227, the results of Tables 1 to 4 above were obtained. In the organic light emitting device of Comparative Example 1, a material that has been widely used conventionally was used.

In Comparative Examples 2 to 61, the organic light emitting devices were manufactured by using the compound of Chemical Formula 2 of the present disclosure as the electron blocking layer, and using a single host as the light emitting layer in the same manner as in Comparative Example 1. When the compound of Chemical Formula 1 and the compound of Chemical Formula 2 were co-deposited and used as the light emitting layer as in Example of Table 1, it was confirmed that the driving voltage decreased and the efficiency and lifetime increased as compared with Comparative Example of Table 2.

Further, when Comparative Example compounds B-1 to B-12 and the compound of Chemical Formula 2 of the present disclosure were co-deposited and used as the light emitting layer as shown in Table 3, the result showed that the driving voltage generally increased and the efficiency and lifetime decreased as compared with a combination of the present disclosure. As shown in Table 4, even when Comparative Example compounds C-1 to C-12 and the compounds of Chemical Formula 1 of the present disclosure were co-deposited and used as the light emitting layer, the result showed that the driving voltage increased and the efficiency and lifetime decreased.

From these results, it can be seen that when the compound of Chemical Formula 1 as the first host and the compound of Chemical Formula 2 as the second host were used in combination, energy transfer to the red dopant in the light emitting layer was well performed, thereby improving the driving voltage and increasing the efficiency and lifetime. In addition, it can be inferred that the combination of the compounds of Examples can form a more stable balance within the light emitting layer than the combination of the compounds of Comparative Examples to form excitons by the combination of electrons and holes, thereby further improving the efficiency and lifetime of the manufactured organic light emitting devices.

That is, when the compound of Chemical Formula 1 and the compound of Chemical Formula 2 of the present disclosure were used in combination as a host of the light emitting layer, the driving voltage, luminous efficiency, and lifetime characteristics of the organic light emitting devices could be improved.

Description of Symbols

| | |
|---|---|
| 1: substrate | 2: anode |
| 3: light emitting layer | 4: cathode |
| 5: hole injection layer | 6: hole transport layer |
| 7: electron blocking layer | 8: hole blocking layer |
| 9: electron injection and transport layer | |

1. An organic light emitting device comprising:
an anode;
a cathode; and
a light emitting layer disposed between the anode and the cathode,
wherein the light emitting layer includes a compound of the following Chemical Formula 1 and a compound of the following Chemical Formula 2: 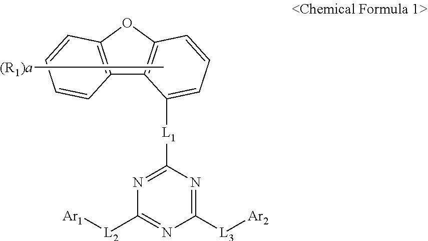
wherein in Chemical Formula 1:
Ar₁ and Ar₂ are each independently a substituted or unsubstituted C₆₋₆₀ aryl or a substituted or unsubstituted C₂₋₆₀ heteroaryl containing one or more selected from the group consisting of N, O and S;
L₁ to L₃ are each independently a single bond or a substituted or unsubstituted C₆₋₆₀ arylene;
R₁ is hydrogen, deuterium, a substituted or unsubstituted C₆₋₆₀ aryl, or a substituted or unsubstituted C₂₋₆₀ heteroaryl containing one or more selected from the group consisting of N, O and S;
a is an integer of 0 to 7; 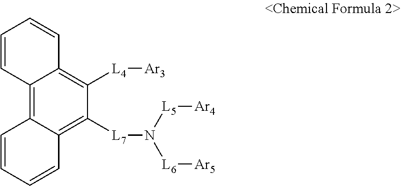
wherein in Chemical Formula 2:
Ar₃ is hydrogen, a substituted or unsubstituted C₆₋₆₀ aryl, or a substituted or unsubstituted C₂₋₆₀ heteroaryl containing one or more selected from the group consisting of N, O and S;
Ar₄ and Ar₅ are each independently a substituted or unsubstituted C₆₋₆₀ aryl or a substituted or unsubstituted C₂₋₆₀ heteroaryl containing one or more selected from the group consisting of N, O and S;
L₄ to L₆ are each independently a single bond, a substituted or unsubstituted C₆₋₆₀ arylene, or a substituted or unsubstituted C₂₋₆₀ heteroarylene containing one or more selected from the group consisting of N, O and S; and
L₇ is a substituted or unsubstituted C₆₋₆₀ arylene.

2. The organic light emitting device according to claim 1,
wherein the compound of Chemical Formula 1 is any one of the following Chemical Formulas 1-1 to 1-3: 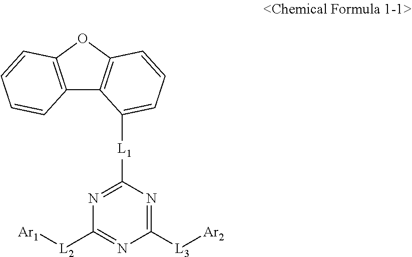 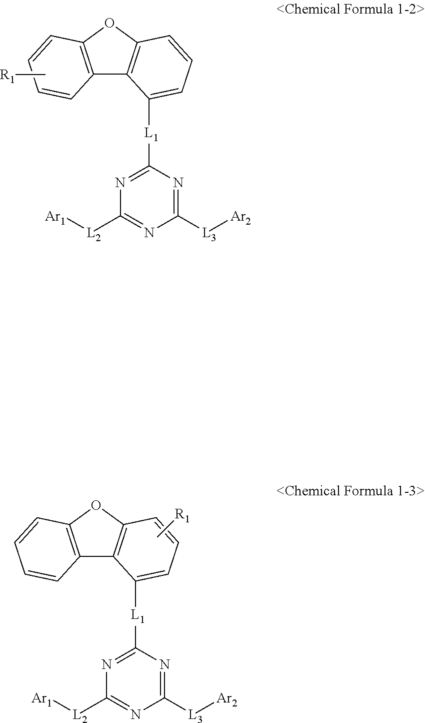
wherein in Chemical Formulas 1-1 to 1-3,
Ar₁ and Ar₂, L₁ to L₃ and R₁ are as defined in claim 1.

3. The organic light emitting device according to claim 1,
wherein Ar₁ and Ar₂ are each independently phenyl, biphenylyl, terphenylyl, naphthyl, phenanthrenyl, dibenzofuranyl, or dibenzothiophenyl.

4. The organic light emitting device according to claim 1,
wherein L₁ to L₃ are each independently a single bond or any one selected from the group consisting of the following.

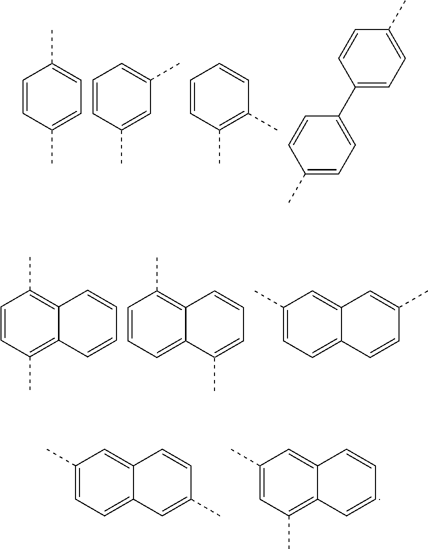

5. The organic light emitting device according to claim 1,
wherein each R₁ is each independently hydrogen, deuterium, phenyl, biphenylyl, terphenylyl, naphthyl, phenanthrenyl, triphenylenyl, naphthyl phenyl, phenyl naphthyl, fluoranthenyl, dihydroindenyl, dibenzofuranyl, dibenzothiophenyl, benzonaphthofuranyl, or benzonaphthothiophenyl.

6. The organic light emitting device according to claim 1,
wherein at least one of Ar₁, Ar₂ and R₁ is naphthyl, phenyl naphthyl, naphthyl phenyl, phenanthrenyl, fluoranthenyl, dibenzofuranyl, dibenzothiophenyl, benzonaphtho-furanyl, or benzonaphthothiophenyl.

7. The organic light emitting device according to claim 1,
wherein a is 0 or 1.

8. The organic light emitting device according to claim 1,
wherein the compound of Chemical Formula 1 is any one selected from the group consisting of the following:

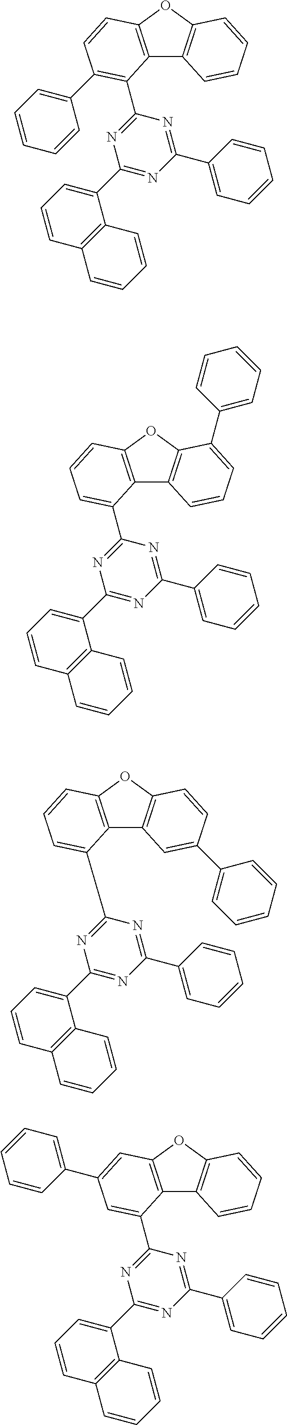 [SMILES: c1ccc(-c2nc(-c3cccc4ccccc34)nc(-c3c(-c4ccccc4)ccc4oc5ccccc5c34)n2)cc1 ; c1ccc(-c2nc(-c3cccc4ccccc34)nc(-c3cccc4oc5c(-c6ccccc6)cccc5c34)n2)cc1 ; c1ccc(-c2ccc3oc4cccc(-c5nc(-c6ccccc6)nc(-c6cccc7ccccc67)n5)c4c3c2)cc1 ; c1ccc(-c2cc(-c3nc(-c4ccccc4)nc(-c4cccc5ccccc45)n3)c3c(c2)oc2ccccc23)cc1]

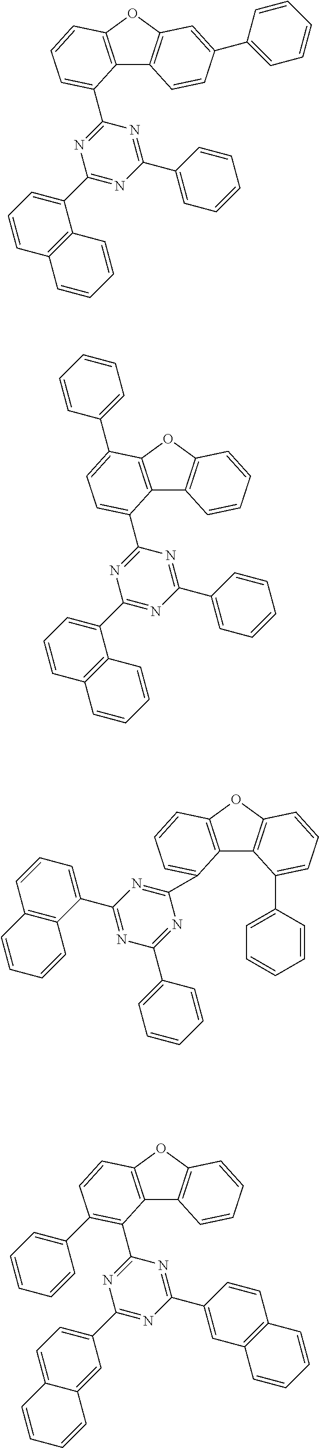 [SMILES: c1ccc(-c2ccc3c(c2)oc2cccc(-c4nc(-c5ccccc5)nc(-c5cccc6ccccc56)n4)c23)cc1 ; c1ccc(-c2nc(-c3cccc4ccccc34)nc(-c3ccc(-c4ccccc4)c4oc5ccccc5c34)n2)cc1 ; c1ccc(-c2nc(-c3cccc4ccccc34)nc(-c3cccc4oc5cccc(-c6ccccc6)c5c34)n2)cc1 ; c1ccc(-c2ccc3oc4ccccc4c3c2-c2nc(-c3ccc4ccccc4c3)nc(-c3ccc4ccccc4c3)n2)cc1]

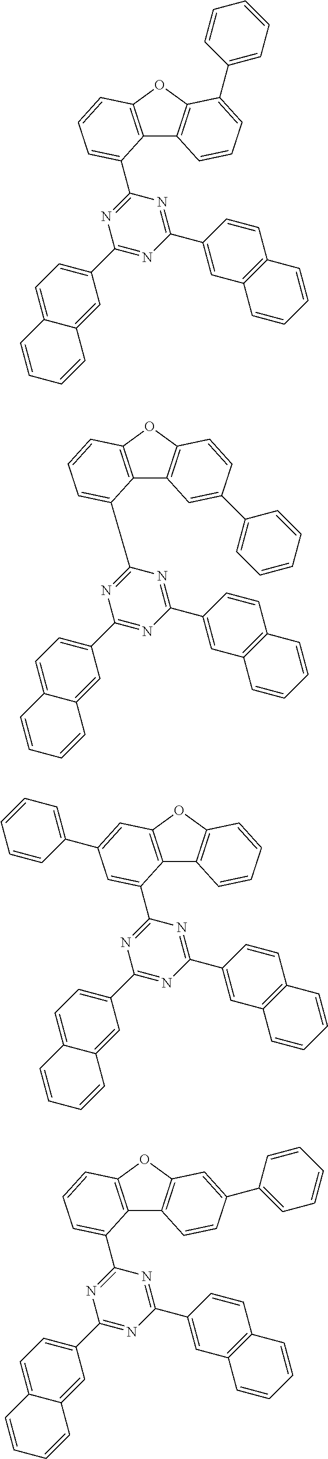 [SMILES: c1ccc(-c2cccc3c2oc2cccc(-c4nc(-c5ccc6ccccc6c5)nc(-c5ccc6ccccc6c5)n4)c23)cc1 ; c1ccc(-c2ccc3oc4cccc(-c5nc(-c6ccc7ccccc7c6)nc(-c6ccc7ccccc7c6)n5)c4c3c2)cc1 ; c1ccc(-c2cc(-c3nc(-c4ccc5ccccc5c4)nc(-c4ccc5ccccc5c4)n3)c3c(c2)oc2ccccc23)cc1 ; c1ccc(-c2ccc3c(c2)oc2cccc(-c4nc(-c5ccc6ccccc6c5)nc(-c5ccc6ccccc6c5)n4)c23)cc1]

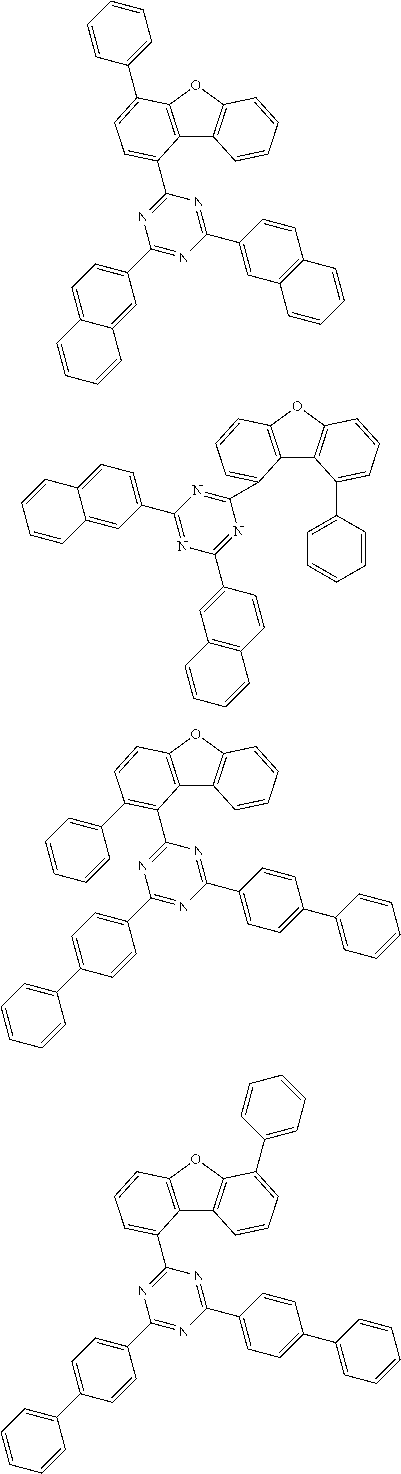

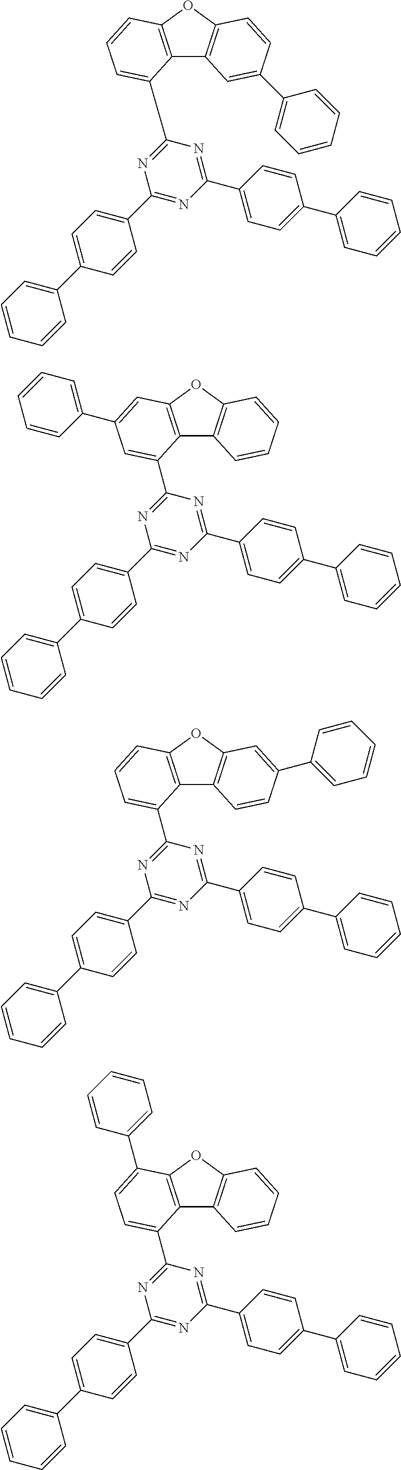

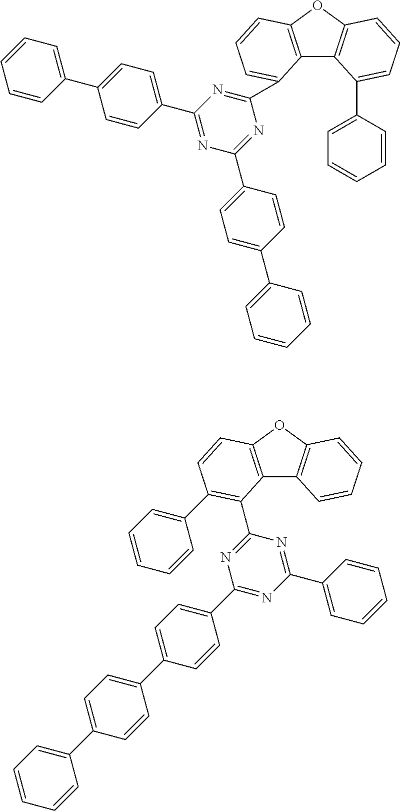 [SMILES: c1ccc(-c2ccc(-c3nc(-c4ccc(-c5ccccc5)cc4)nc(-c4cccc5oc6cccc(-c7ccccc7)c6c45)n3)cc2)cc1 ; c1ccc(-c2ccc(-c3ccc(-c4nc(-c5ccccc5)nc(-c5c(-c6ccccc6)ccc6oc7ccccc7c56)n4)cc3)cc2)cc1]

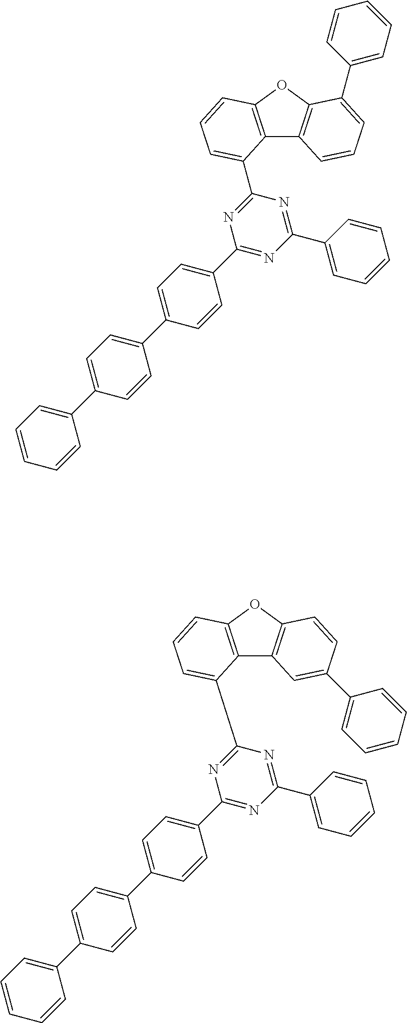 [SMILES: c1ccc(-c2ccc(-c3ccc(-c4nc(-c5ccccc5)nc(-c5cccc6oc7c(-c8ccccc8)cccc7c56)n4)cc3)cc2)cc1 ; c1ccc(-c2ccc(-c3ccc(-c4nc(-c5ccccc5)nc(-c5cccc6oc7ccc(-c8ccccc8)cc7c56)n4)cc3)cc2)cc1]

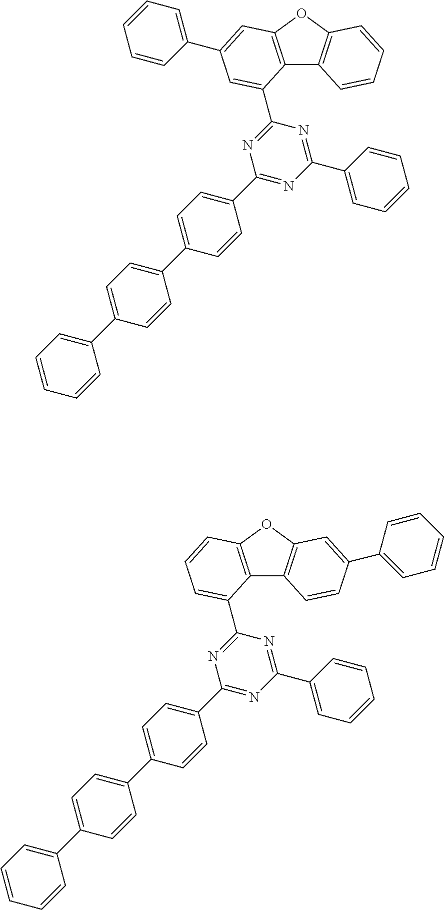 [SMILES: c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccccc8)ccc7c56)n4)cc3)cc2)cc1]

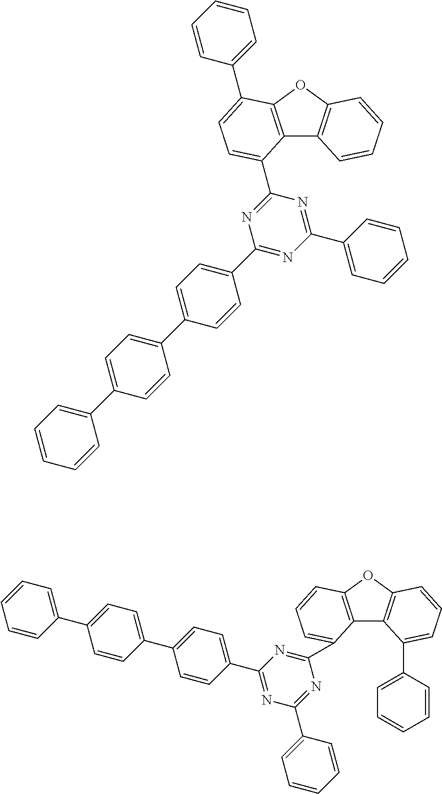 [SMILES: c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1]

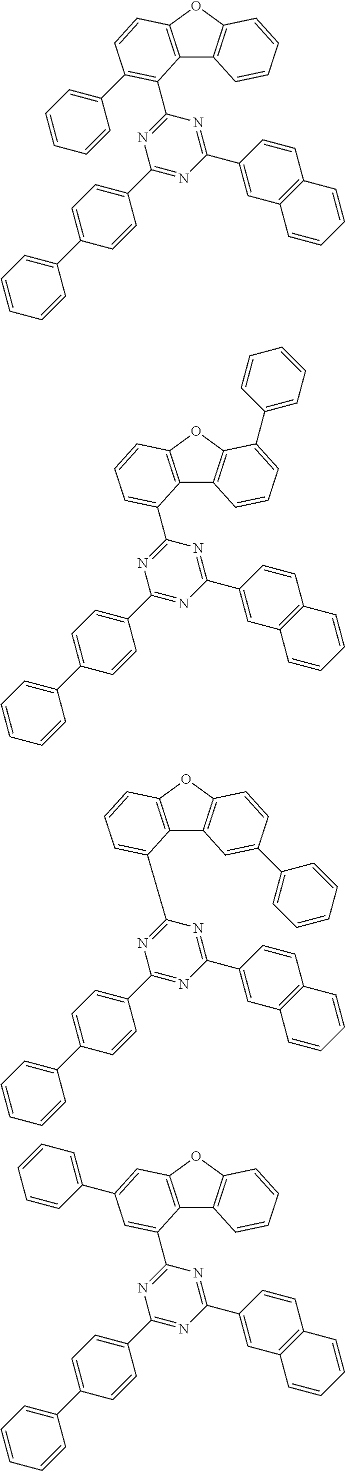

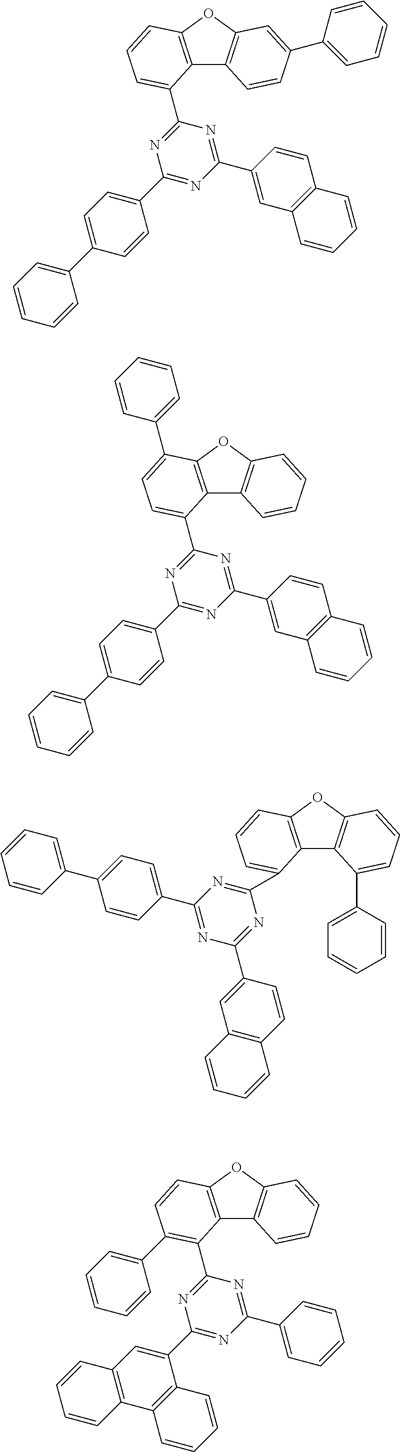 [SMILES: c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc(-c3nc(-c4ccc5ccccc5c4)nc(-c4ccc(-c5ccccc5)c5oc6ccccc6c45)n3)cc2)cc1 ; c1ccc(-c2ccc(-c3nc(-c4ccc5ccccc5c4)nc(-c4cccc5oc6cccc(-c7ccccc7)c6c45)n3)cc2)cc1 ; c1ccc(-c2nc(-c3cc4ccccc4c4ccccc34)nc(-c3c(-c4ccccc4)ccc4oc5ccccc5c34)n2)cc1]

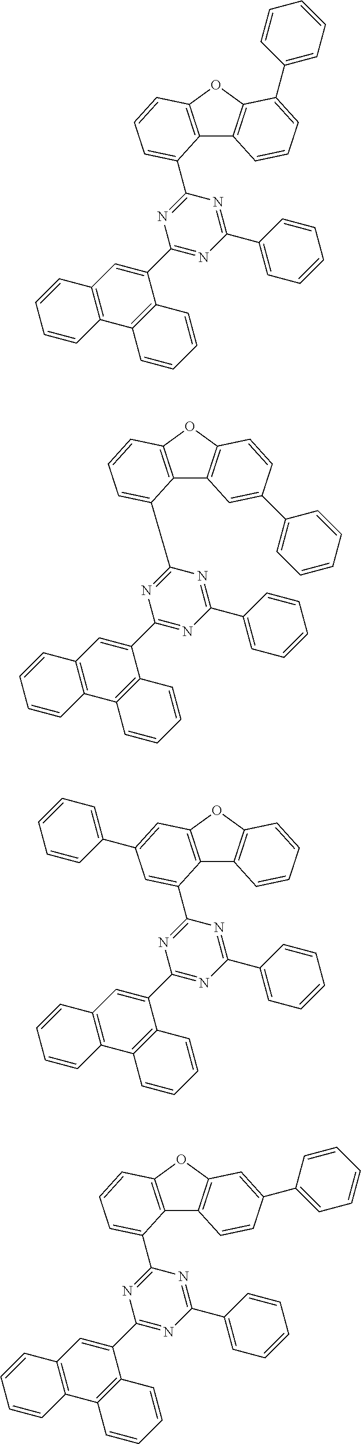 [SMILES: c1ccc(-c2nc(-c3cc4ccccc4c4ccccc34)nc(-c3cccc4oc5c(-c6ccccc6)cccc5c34)n2)cc1 ; c1ccc(-c2ccc3oc4cccc(-c5nc(-c6ccccc6)nc(-c6cc7ccccc7c7ccccc67)n5)c4c3c2)cc1 ; c1ccc(-c2cc(-c3nc(-c4ccccc4)nc(-c4cc5ccccc5c5ccccc45)n3)c3c(c2)oc2ccccc23)cc1 ; c1ccc(-c2ccc3c(c2)oc2cccc(-c4nc(-c5ccccc5)nc(-c5cc6ccccc6c6ccccc56)n4)c23)cc1]

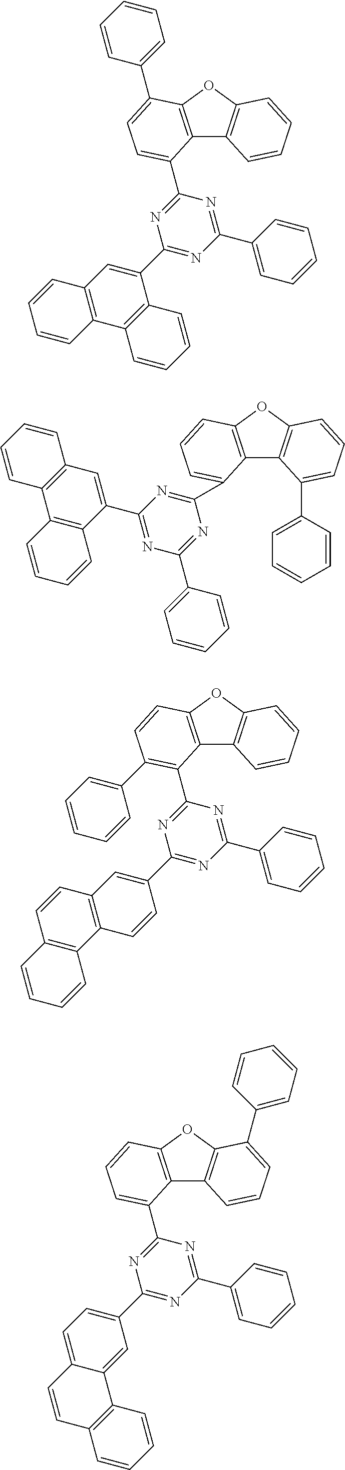

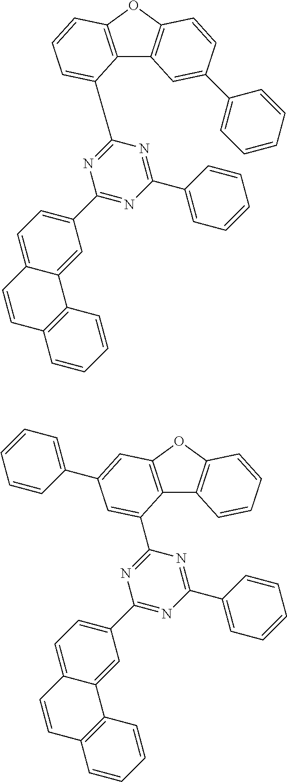 [SMILES: c1ccc(-c2ccc3oc4cccc(-c5nc(-c6ccccc6)nc(-c6ccc7ccc8ccccc8c7c6)n5)c4c3c2)cc1 ; c1ccc(-c2cc(-c3nc(-c4ccccc4)nc(-c4ccc5ccc6ccccc6c5c4)n3)c3c(c2)oc2ccccc23)cc1]

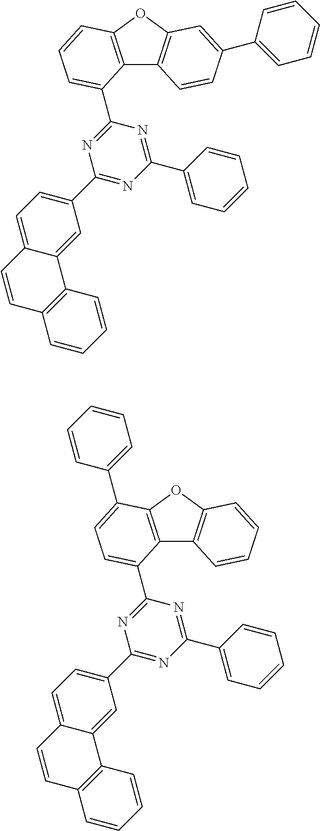 [SMILES: c1ccc(-c2ccc3c(c2)oc2cccc(-c4nc(-c5ccccc5)nc(-c5ccc6ccc7ccccc7c6c5)n4)c23)cc1 ; c1ccc(-c2nc(-c3ccc4ccc5ccccc5c4c3)nc(-c3ccc(-c4ccccc4)c4oc5ccccc5c34)n2)cc1]

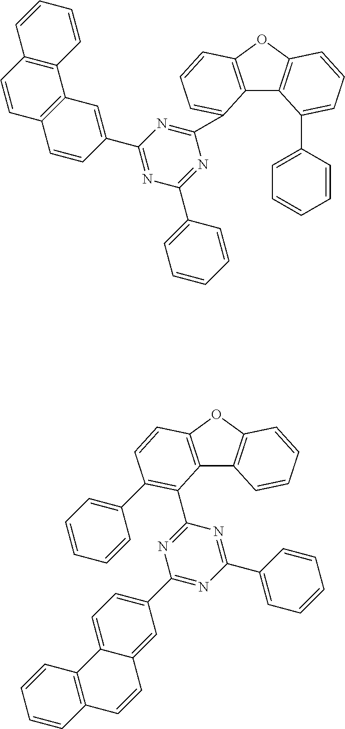 [SMILES: c1ccc(-c2nc(-c3ccc4ccc5ccccc5c4c3)nc(-c3cccc4oc5cccc(-c6ccccc6)c5c34)n2)cc1 ; c1ccc(-c2nc(-c3ccc4c(ccc5ccccc54)c3)nc(-c3c(-c4ccccc4)ccc4oc5ccccc5c34)n2)cc1]

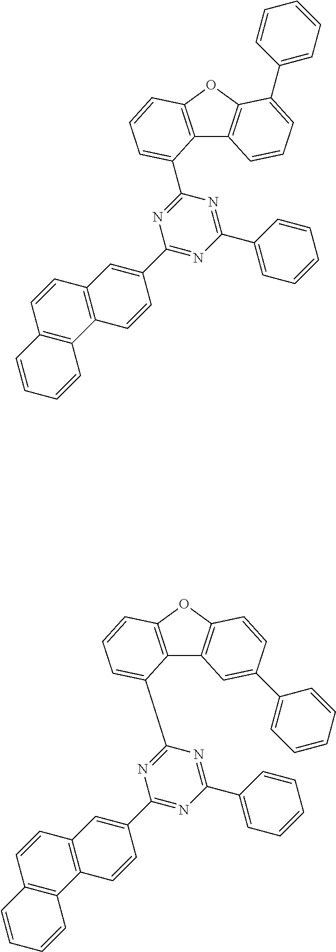 [SMILES: c1ccc(-c2nc(-c3ccc4c(ccc5ccccc54)c3)nc(-c3cccc4oc5c(-c6ccccc6)cccc5c34)n2)cc1 ; c1ccc(-c2ccc3oc4cccc(-c5nc(-c6ccccc6)nc(-c6ccc7c(ccc8ccccc87)c6)n5)c4c3c2)cc1]

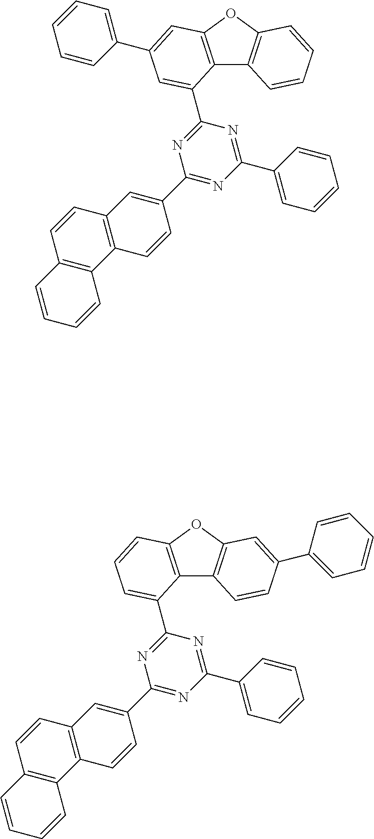 [SMILES: c1ccc(-c2cc(-c3nc(-c4ccccc4)nc(-c4ccc5c(ccc6ccccc65)c4)n3)c3c(c2)oc2ccccc23)cc1 ; c1ccc(-c2ccc3c(c2)oc2cccc(-c4nc(-c5ccccc5)nc(-c5ccc6c(ccc7ccccc76)c5)n4)c23)cc1]

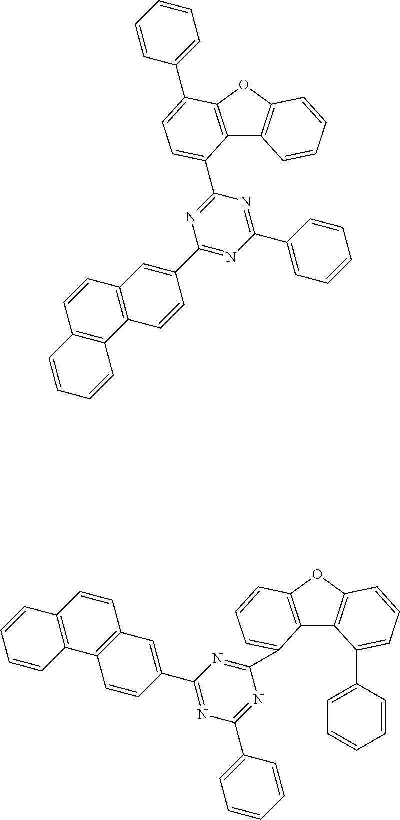 [SMILES: c1ccc(-c2nc(-c3ccc4c(ccc5ccccc54)c3)nc(-c3ccc(-c4ccccc4)c4oc5ccccc5c34)n2)cc1 ; c1ccc(-c2nc(-c3ccc4c(ccc5ccccc54)c3)nc(-c3cccc4oc5cccc(-c6ccccc6)c5c34)n2)cc1]

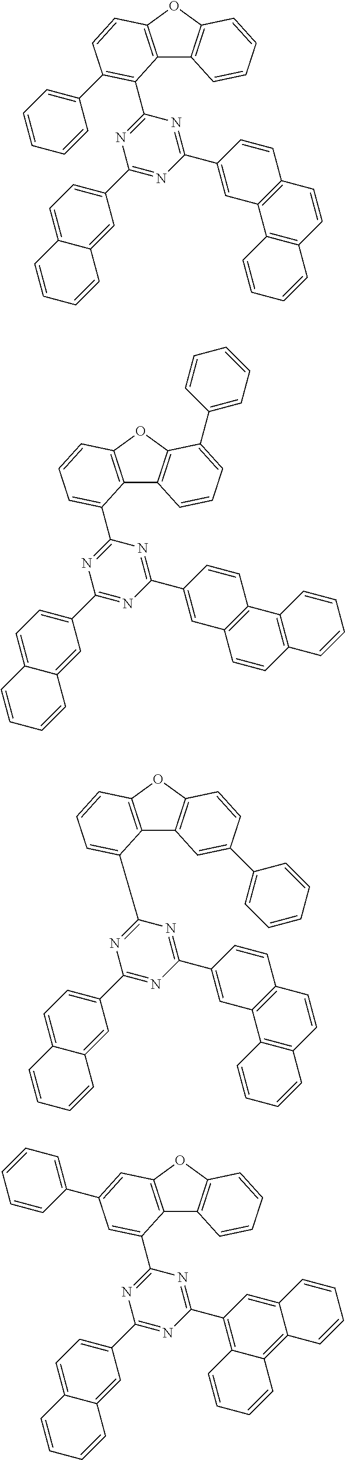 [SMILES: c1ccc(-c2ccc3oc4ccccc4c3c2-c2nc(-c3ccc4ccccc4c3)nc(-c3ccc4ccc5ccccc5c4c3)n2)cc1 ; c1ccc(-c2cccc3c2oc2cccc(-c4nc(-c5ccc6ccccc6c5)nc(-c5ccc6c(ccc7ccccc76)c5)n4)c23)cc1 ; c1ccc(-c2ccc3oc4cccc(-c5nc(-c6ccc7ccccc7c6)nc(-c6ccc7ccc8ccccc8c7c6)n5)c4c3c2)cc1 ; c1ccc(-c2cc(-c3nc(-c4ccc5ccccc5c4)nc(-c4cc5ccccc5c5ccccc45)n3)c3c(c2)oc2ccccc23)cc1]

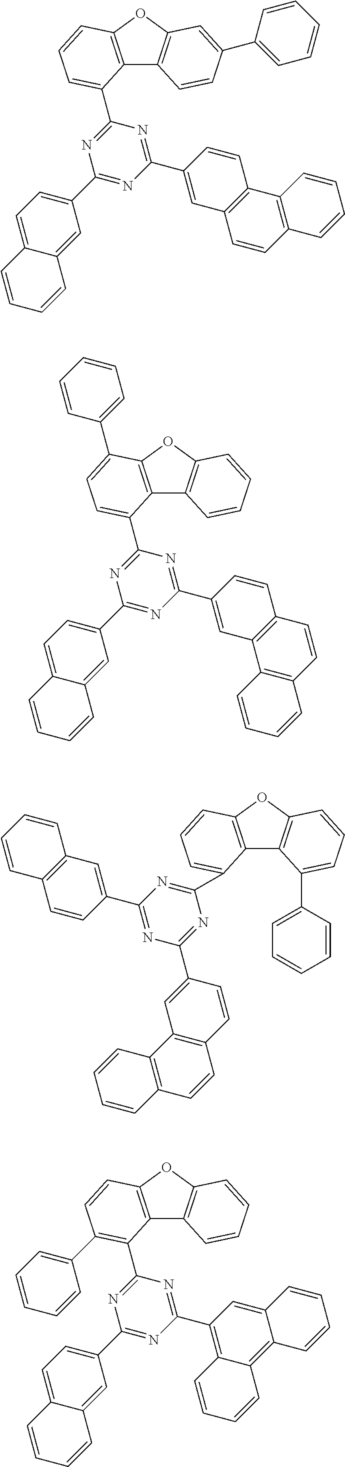 [SMILES: c1ccc(-c2ccc3c(c2)oc2cccc(-c4nc(-c5ccc6ccccc6c5)nc(-c5ccc6c(ccc7ccccc76)c5)n4)c23)cc1 ; c1ccc(-c2ccc(-c3nc(-c4ccc5ccccc5c4)nc(-c4ccc5ccc6ccccc6c5c4)n3)c3c2oc2ccccc23)cc1 ; c1ccc(-c2cccc3oc4cccc(-c5nc(-c6ccc7ccccc7c6)nc(-c6ccc7ccc8ccccc8c7c6)n5)c4c23)cc1 ; c1ccc(-c2ccc3oc4ccccc4c3c2-c2nc(-c3ccc4ccccc4c3)nc(-c3cc4ccccc4c4ccccc34)n2)cc1]

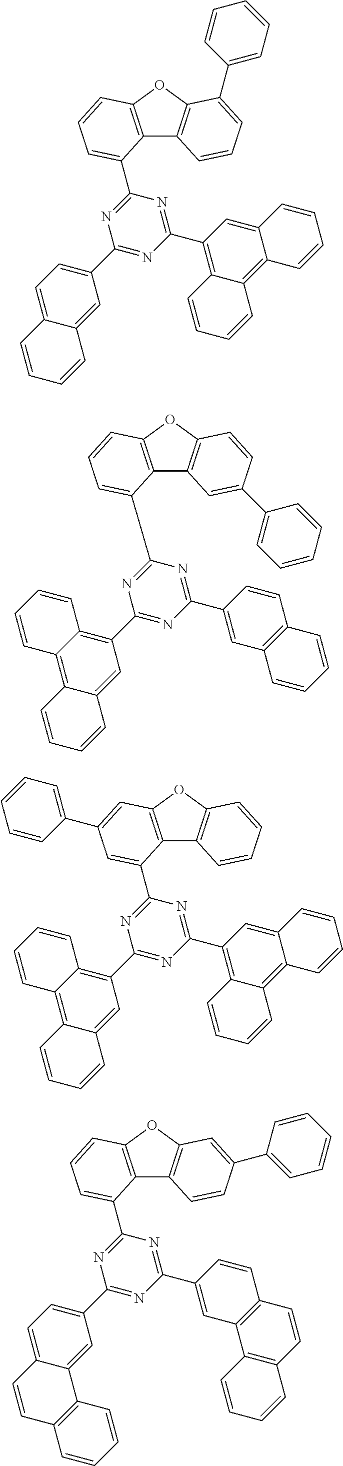

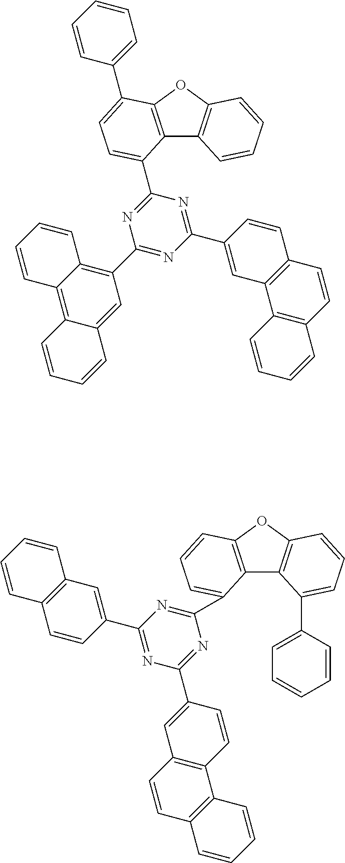 [SMILES: c1ccc(-c2ccc(-c3nc(-c4ccc5ccc6ccccc6c5c4)nc(-c4cc5ccccc5c5ccccc45)n3)c3c2oc2ccccc23)cc1 ; c1ccc(-c2cccc3oc4cccc(-c5nc(-c6ccc7ccccc7c6)nc(-c6ccc7c(ccc8ccccc87)c6)n5)c4c23)cc1]

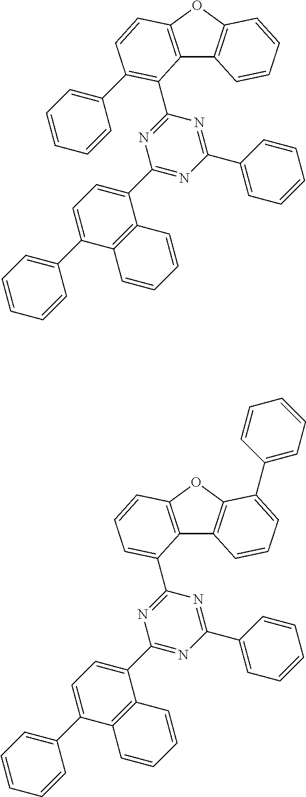 [SMILES: c1ccc(-c2nc(-c3ccc(-c4ccccc4)c4ccccc34)nc(-c3c(-c4ccccc4)ccc4oc5ccccc5c34)n2)cc1 ; c1ccc(-c2nc(-c3ccc(-c4ccccc4)c4ccccc34)nc(-c3cccc4oc5c(-c6ccccc6)cccc5c34)n2)cc1]

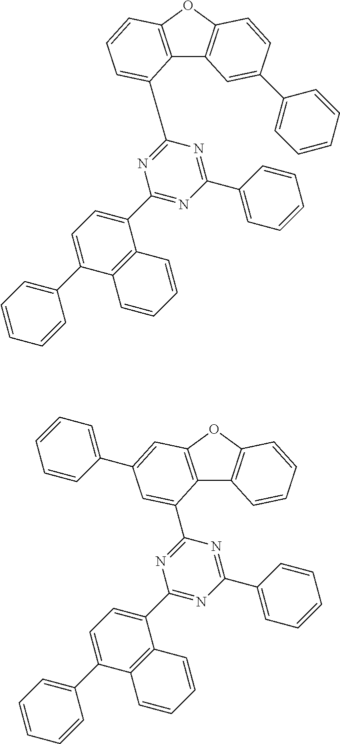 [SMILES: c1ccc(-c2ccc3oc4cccc(-c5nc(-c6ccccc6)nc(-c6ccc(-c7ccccc7)c7ccccc67)n5)c4c3c2)cc1 ; c1ccc(-c2cc(-c3nc(-c4ccccc4)nc(-c4ccc(-c5ccccc5)c5ccccc45)n3)c3c(c2)oc2ccccc23)cc1]

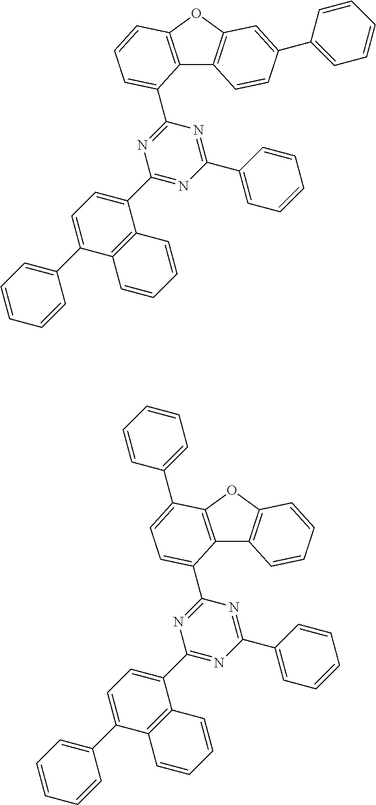 [SMILES: c1ccc(-c2ccc3c(c2)oc2cccc(-c4nc(-c5ccccc5)nc(-c5ccc(-c6ccccc6)c6ccccc56)n4)c23)cc1 ; c1ccc(-c2nc(-c3ccc(-c4ccccc4)c4ccccc34)nc(-c3ccc(-c4ccccc4)c4oc5ccccc5c34)n2)cc1]

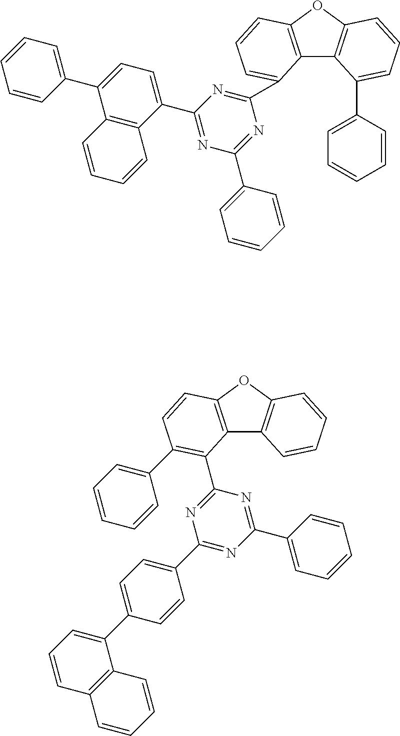 [SMILES: c1ccc(-c2nc(-c3ccc(-c4ccccc4)c4ccccc34)nc(-c3cccc4oc5cccc(-c6ccccc6)c5c34)n2)cc1 ; c1ccc(-c2nc(-c3ccc(-c4cccc5ccccc45)cc3)nc(-c3c(-c4ccccc4)ccc4oc5ccccc5c34)n2)cc1]

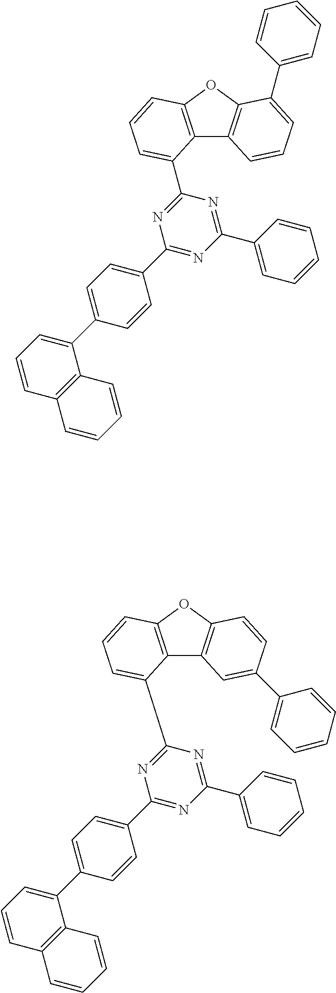 [SMILES: c1ccc(-c2nc(-c3ccc(-c4cccc5ccccc45)cc3)nc(-c3cccc4oc5c(-c6ccccc6)cccc5c34)n2)cc1 ; c1ccc(-c2ccc3oc4cccc(-c5nc(-c6ccccc6)nc(-c6ccc(-c7cccc8ccccc78)cc6)n5)c4c3c2)cc1]

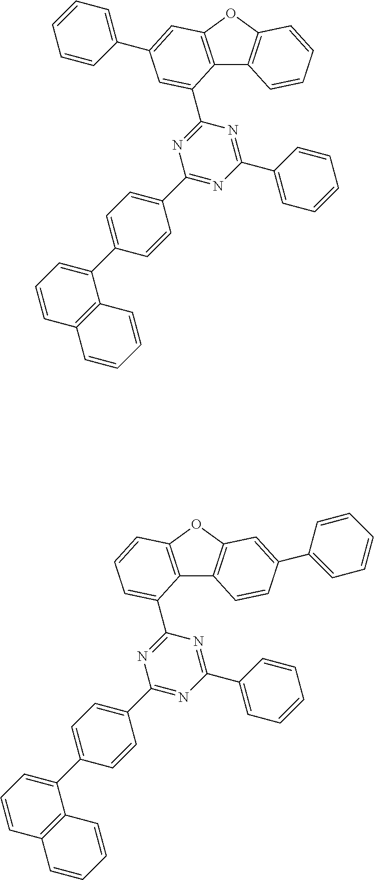 [SMILES: c1ccc(-c2cc(-c3nc(-c4ccccc4)nc(-c4ccc(-c5cccc6ccccc56)cc4)n3)c3c(c2)oc2ccccc23)cc1 ; c1ccc(-c2ccc3c(c2)oc2cccc(-c4nc(-c5ccccc5)nc(-c5ccc(-c6cccc7ccccc67)cc5)n4)c23)cc1]

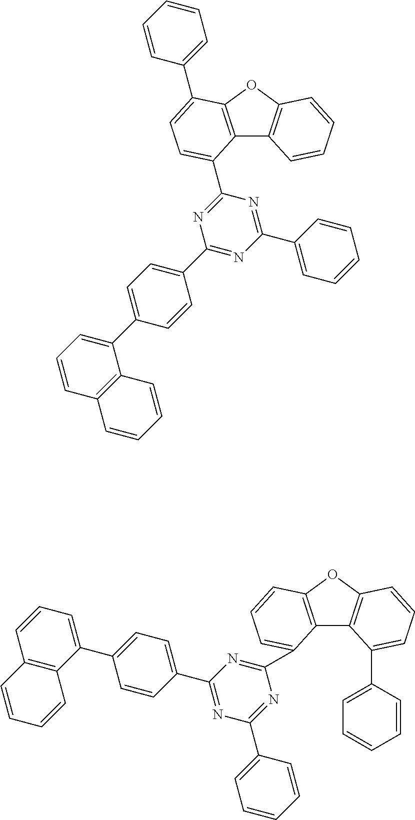 [SMILES: c1ccc(-c2nc(-c3ccc(-c4cccc5ccccc45)cc3)nc(-c3ccc(-c4ccccc4)c4oc5ccccc5c34)n2)cc1 ; c1ccc(-c2nc(-c3ccc(-c4cccc5ccccc45)cc3)nc(-c3cccc4oc5cccc(-c6ccccc6)c5c34)n2)cc1]

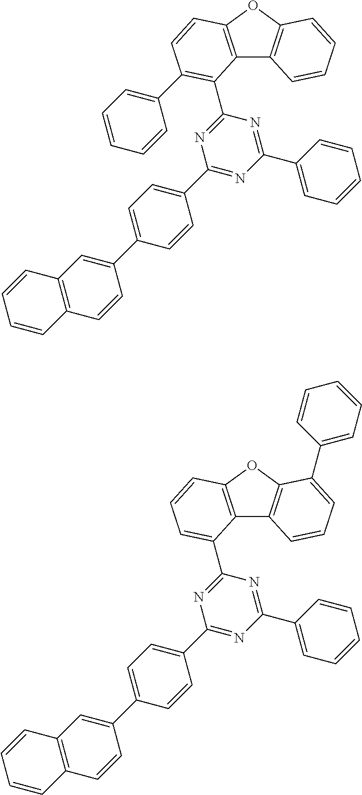 [SMILES: c1ccc(-c2nc(-c3ccc(-c4ccc5ccccc5c4)cc3)nc(-c3c(-c4ccccc4)ccc4oc5ccccc5c34)n2)cc1 ; c1ccc(-c2nc(-c3ccc(-c4ccc5ccccc5c4)cc3)nc(-c3cccc4oc5c(-c6ccccc6)cccc5c34)n2)cc1]

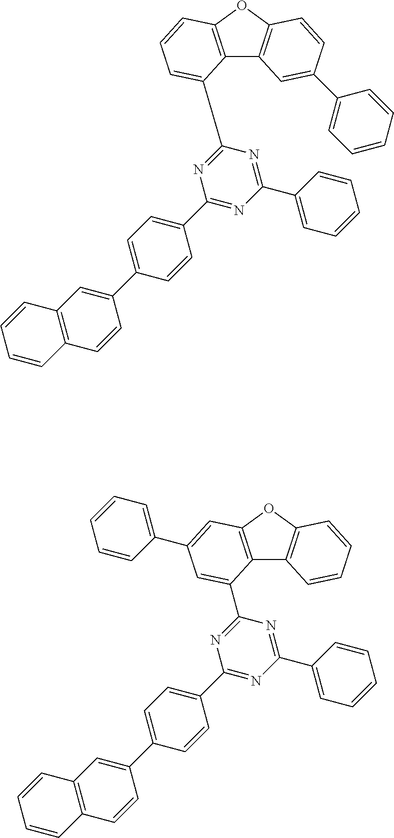 [SMILES: c1ccc(-c2ccc3oc4cccc(-c5nc(-c6ccccc6)nc(-c6ccc(-c7ccc8ccccc8c7)cc6)n5)c4c3c2)cc1 ; c1ccc(-c2cc(-c3nc(-c4ccccc4)nc(-c4ccc(-c5ccc6ccccc6c5)cc4)n3)c3c(c2)oc2ccccc23)cc1]

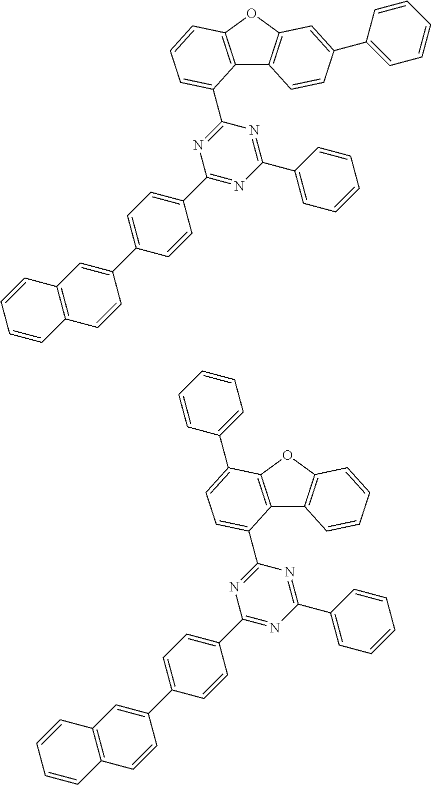 [SMILES: c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2nc(-c3ccc(-c4ccc5ccccc5c4)cc3)nc(-c3ccc(-c4ccccc4)c4oc5ccccc5c34)n2)cc1]

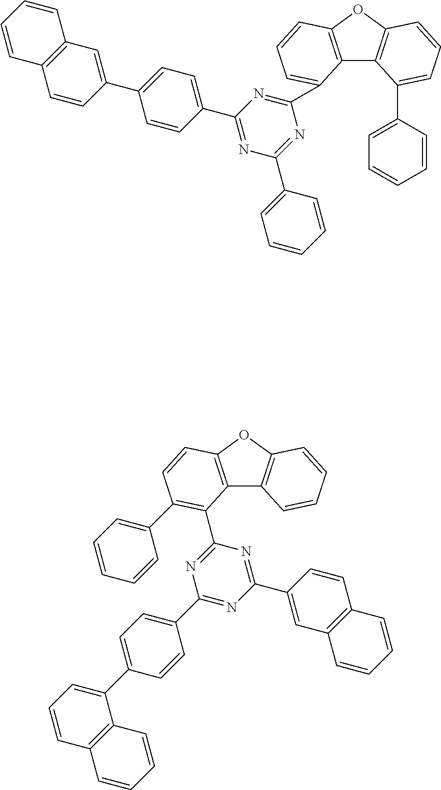 [SMILES: c1ccc(-c2nc(-c3ccc(-c4ccc5ccccc5c4)cc3)nc(-c3cccc4oc5cccc(-c6ccccc6)c5c34)n2)cc1 ; c1ccc(-c2ccc3oc4ccccc4c3c2-c2nc(-c3ccc(-c4cccc5ccccc45)cc3)nc(-c3ccc4ccccc4c3)n2)cc1]

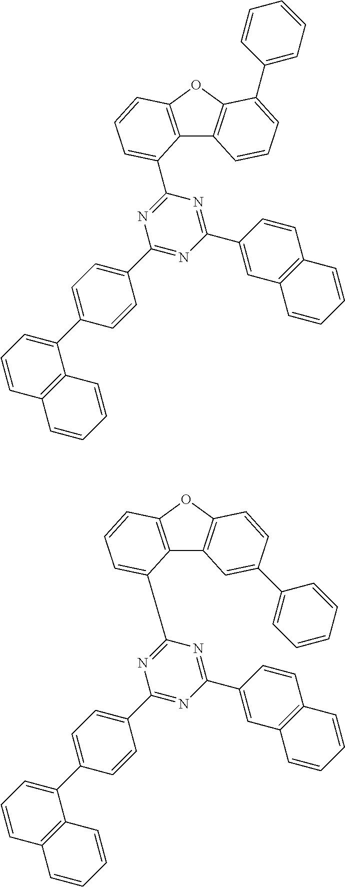 [SMILES: c1ccc(-c2cccc3c2oc2cccc(-c4nc(-c5ccc(-c6cccc7ccccc67)cc5)nc(-c5ccc6ccccc6c5)n4)c23)cc1 ; c1ccc(-c2ccc3oc4cccc(-c5nc(-c6ccc(-c7cccc8ccccc78)cc6)nc(-c6ccc7ccccc7c6)n5)c4c3c2)cc1]

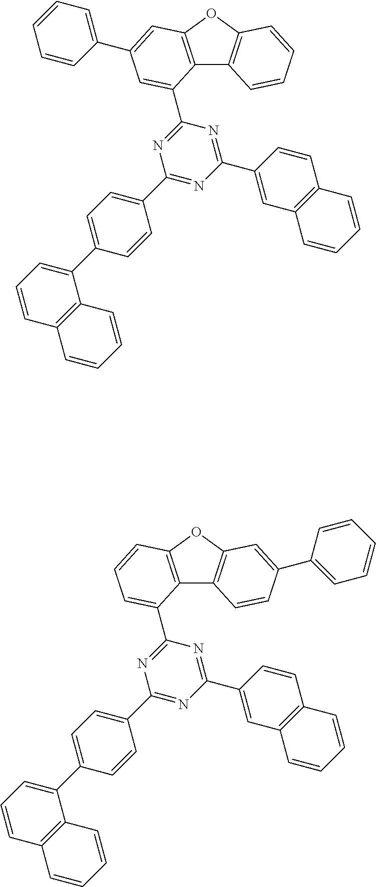 [SMILES: c1ccc(-c2cc(-c3nc(-c4ccc(-c5cccc6ccccc56)cc4)nc(-c4ccc5ccccc5c4)n3)c3c(c2)oc2ccccc23)cc1 ; c1ccc(-c2ccc3c(c2)oc2cccc(-c4nc(-c5ccc(-c6cccc7ccccc67)cc5)nc(-c5ccc6ccccc6c5)n4)c23)cc1]

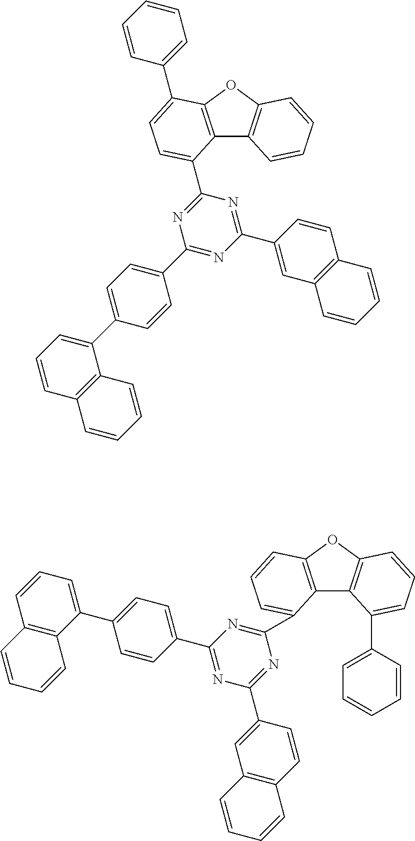 [SMILES: c1ccc(-c2ccc(-c3nc(-c4ccc(-c5cccc6ccccc56)cc4)nc(-c4ccc5ccccc5c4)n3)c3c2oc2ccccc23)cc1 ; c1ccc(-c2cccc3oc4cccc(-c5nc(-c6ccc(-c7cccc8ccccc78)cc6)nc(-c6ccc7ccccc7c6)n5)c4c23)cc1]

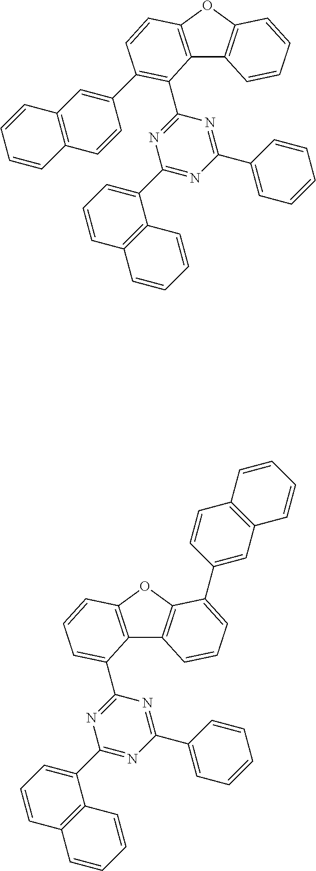 [SMILES: c1ccc(-c2nc(-c3cccc4ccccc34)nc(-c3c(-c4ccc5ccccc5c4)ccc4oc5ccccc5c34)n2)cc1 ; c1ccc(-c2nc(-c3cccc4ccccc34)nc(-c3cccc4oc5c(-c6ccc7ccccc7c6)cccc5c34)n2)cc1]

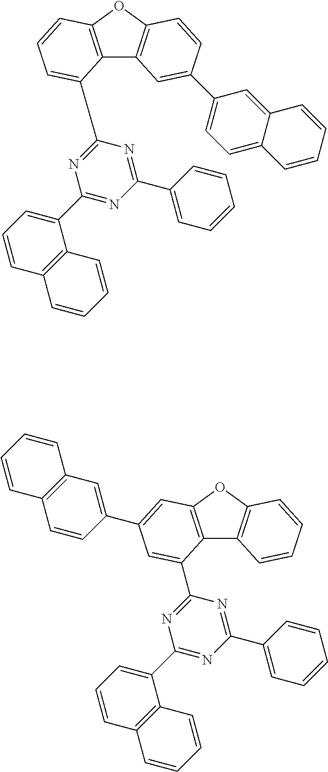 [SMILES: c1ccc(-c2nc(-c3cccc4ccccc34)nc(-c3cccc4oc5ccc(-c6ccc7ccccc7c6)cc5c34)n2)cc1 ; c1ccc(-c2nc(-c3cccc4ccccc34)nc(-c3cc(-c4ccc5ccccc5c4)cc4oc5ccccc5c34)n2)cc1]

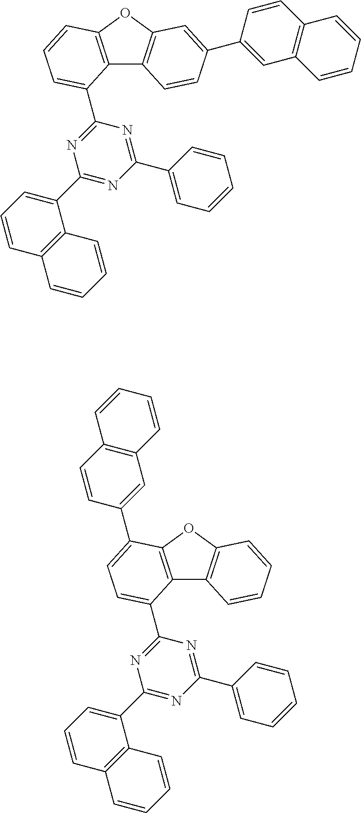 [SMILES: c1ccc(-c2nc(-c3cccc4ccccc34)nc(-c3cccc4oc5cc(-c6ccc7ccccc7c6)ccc5c34)n2)cc1 ; c1ccc(-c2nc(-c3cccc4ccccc34)nc(-c3ccc(-c4ccc5ccccc5c4)c4oc5ccccc5c34)n2)cc1]

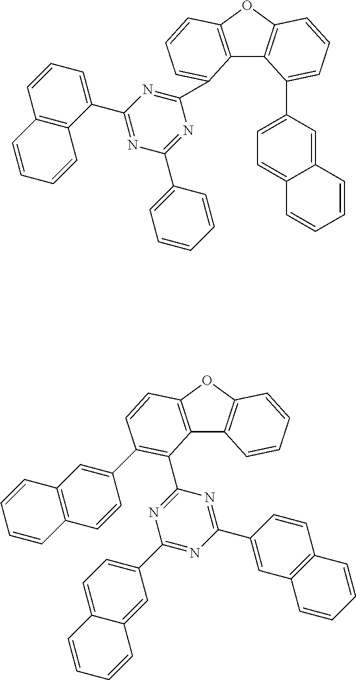 [SMILES: c1ccc(-c2nc(-c3cccc4ccccc34)nc(-c3cccc4oc5cccc(-c6ccc7ccccc7c6)c5c34)n2)cc1 ; c1ccc2cc(-c3nc(-c4ccc5ccccc5c4)nc(-c4c(-c5ccc6ccccc6c5)ccc5oc6ccccc6c45)n3)ccc2c1]

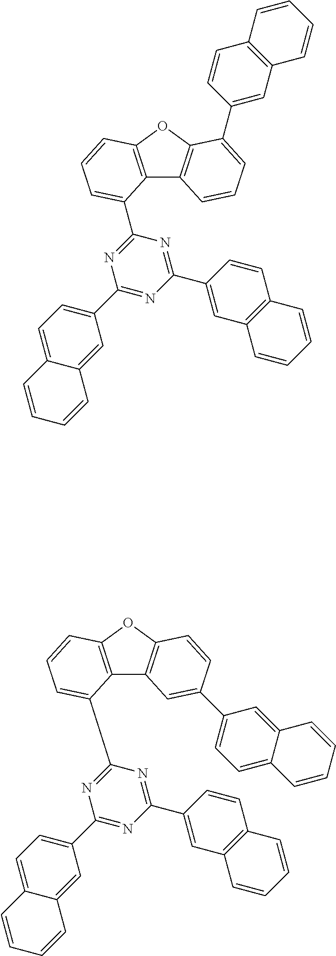 [SMILES: c1ccc2cc(-c3nc(-c4ccc5ccccc5c4)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7ccc8ccccc8c7)n6)c5c4c3)ccc2c1]

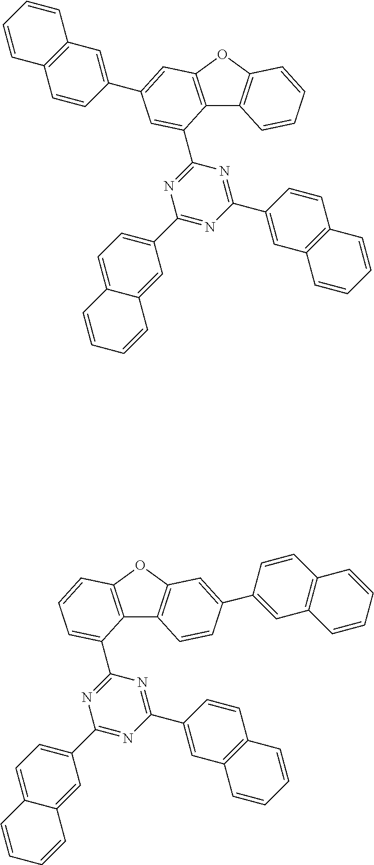 [SMILES: c1ccc2cc(-c3cc(-c4nc(-c5ccc6ccccc6c5)nc(-c5ccc6ccccc6c5)n4)c4c(c3)oc3ccccc34)ccc2c1 ; c1ccc2cc(-c3ccc4c(c3)oc3cccc(-c5nc(-c6ccc7ccccc7c6)nc(-c6ccc7ccccc7c6)n5)c34)ccc2c1]

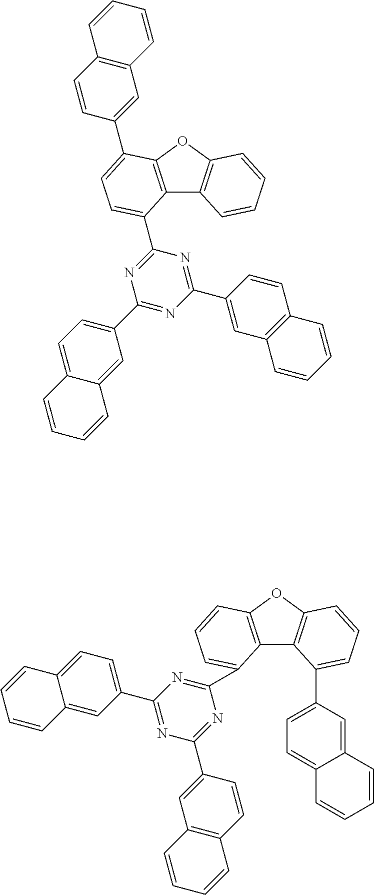 [SMILES: c1ccc2cc(-c3nc(-c4ccc5ccccc5c4)nc(-c4ccc(-c5ccc6ccccc6c5)c5oc6ccccc6c45)n3)ccc2c1 ; c1ccc2cc(-c3nc(-c4ccc5ccccc5c4)nc(-c4cccc5oc6cccc(-c7ccc8ccccc8c7)c6c45)n3)ccc2c1]

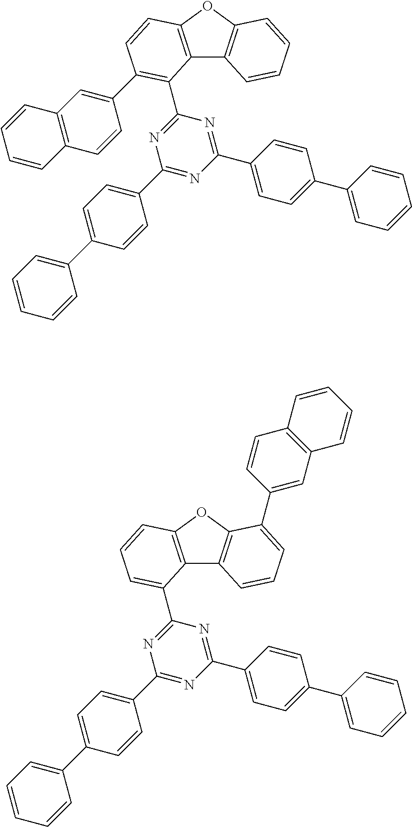 [SMILES: c1ccc(-c2ccc(-c3nc(-c4ccc(-c5ccccc5)cc4)nc(-c4c(-c5ccc6ccccc6c5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc8ccccc8c7)cccc6c45)n3)cc2)cc1]

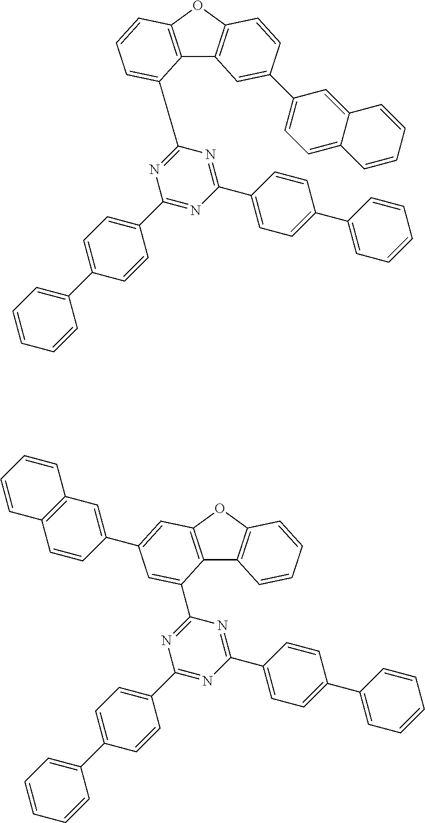 [SMILES: c1ccc(-c2ccc(-c3nc(-c4ccc(-c5ccccc5)cc4)nc(-c4cccc5oc6ccc(-c7ccc8ccccc8c7)cc6c45)n3)cc2)cc1 ; c1ccc(-c2ccc(-c3nc(-c4ccc(-c5ccccc5)cc4)nc(-c4cc(-c5ccc6ccccc6c5)cc5oc6ccccc6c45)n3)cc2)cc1]

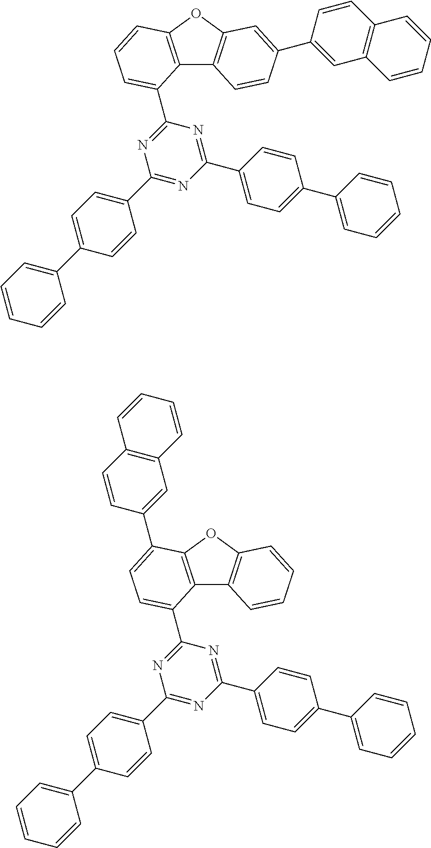 [SMILES: c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1]

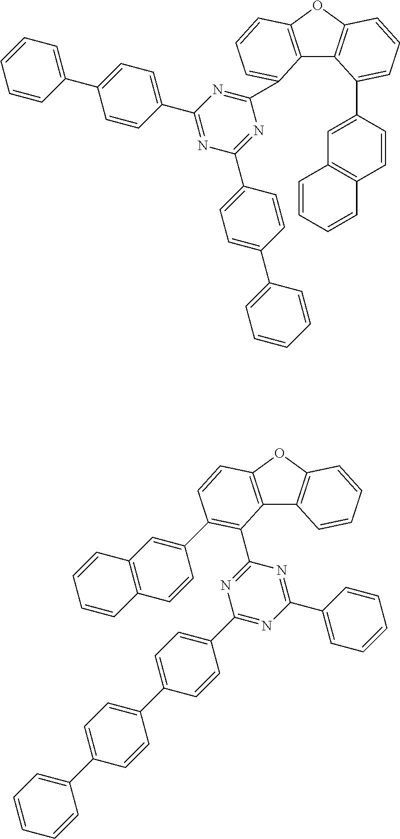 [SMILES: c1ccc(-c2ccc(-c3nc(-c4ccc(-c5ccccc5)cc4)nc(-c4cccc5oc6cccc(-c7ccc8ccccc8c7)c6c45)n3)cc2)cc1 ; c1ccc(-c2ccc(-c3ccc(-c4nc(-c5ccccc5)nc(-c5c(-c6ccc7ccccc7c6)ccc6oc7ccccc7c56)n4)cc3)cc2)cc1]

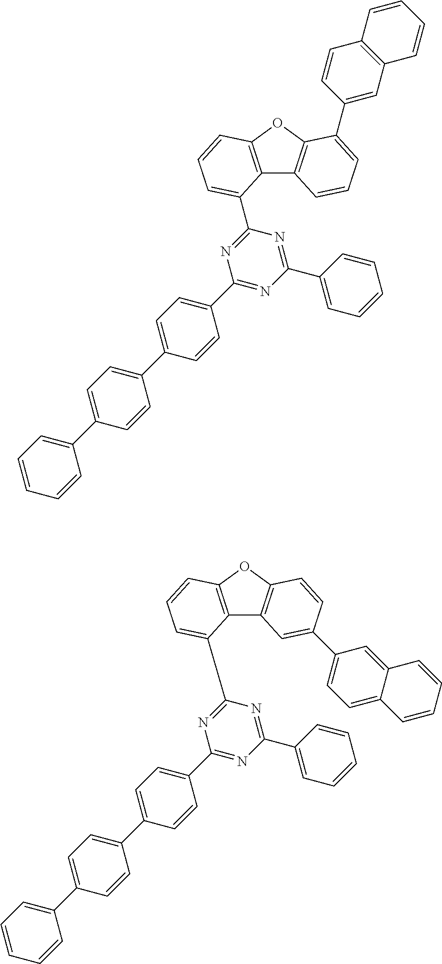 [SMILES: c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1]

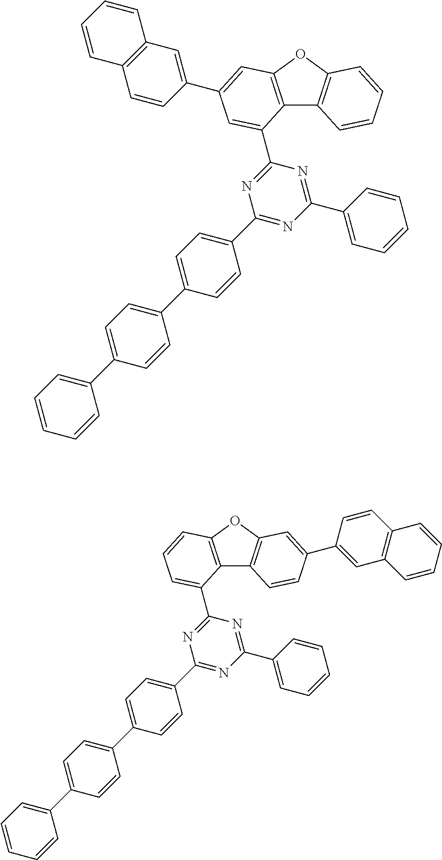 [SMILES: c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1]

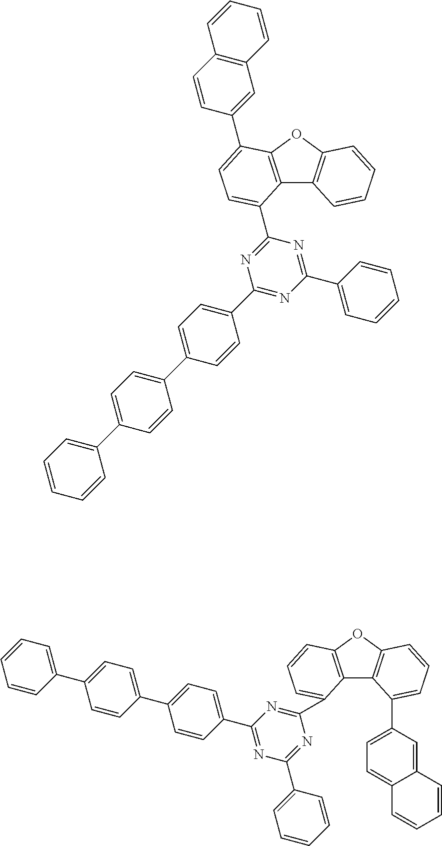 [SMILES: c1ccc(-c2ccc(-c3ccc(-c4nc(-c5ccccc5)nc(-c5ccc(-c6ccc7ccccc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1]

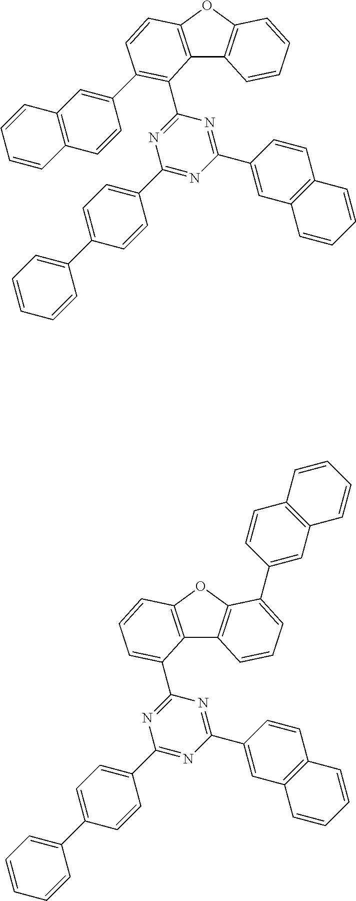 [SMILES: c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2ccc(-c3nc(-c4ccc5ccccc5c4)nc(-c4cccc5oc6c(-c7ccc8ccccc8c7)cccc6c45)n3)cc2)cc1]

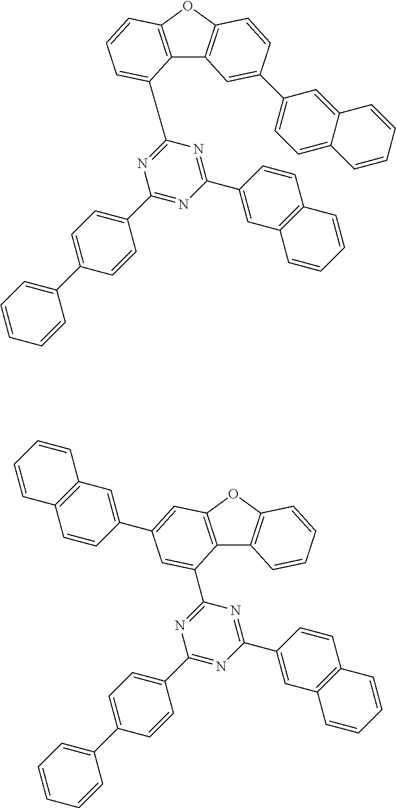 [SMILES: c1ccc(-c2ccc(-c3nc(-c4ccc5ccccc5c4)nc(-c4cccc5oc6ccc(-c7ccc8ccccc8c7)cc6c45)n3)cc2)cc1 ; c1ccc(-c2ccc(-c3nc(-c4ccc5ccccc5c4)nc(-c4cc(-c5ccc6ccccc6c5)cc5oc6ccccc6c45)n3)cc2)cc1]

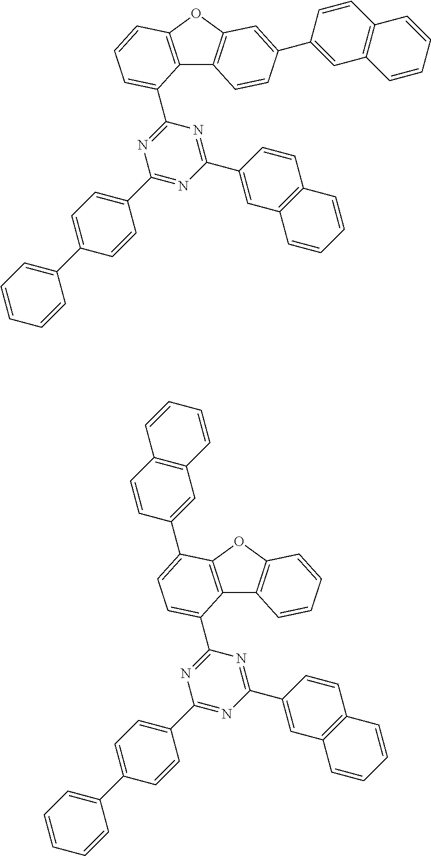 [SMILES: c1ccc(-c2ccc(-c3nc(-c4ccc5ccccc5c4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4ccc(-c5ccc6ccccc6c5)c5oc6ccccc6c45)n3)cc2)cc1]

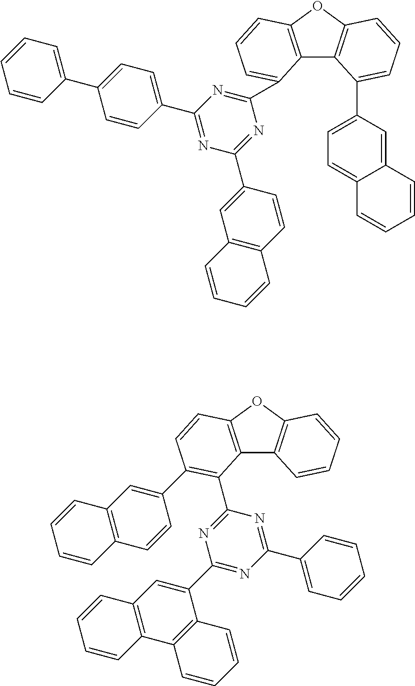 [SMILES: c1ccc(-c2ccc(-c3nc(-c4ccc5ccccc5c4)nc(-c4cccc5oc6cccc(-c7ccc8ccccc8c7)c6c45)n3)cc2)cc1 ; c1ccc(-c2nc(-c3cc4ccccc4c4ccccc34)nc(-c3c(-c4ccc5ccccc5c4)ccc4oc5ccccc5c34)n2)cc1]

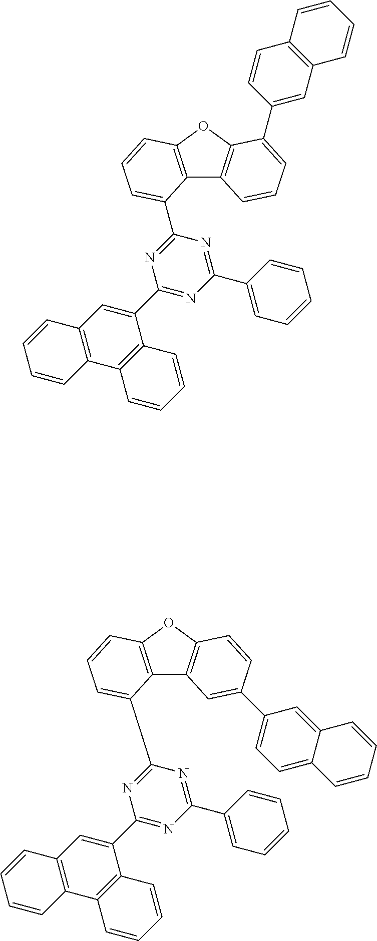 [SMILES: c1ccc(-c2nc(-c3cc4ccccc4c4ccccc34)nc(-c3cccc4oc5c(-c6ccc7ccccc7c6)cccc5c34)n2)cc1 ; c1ccc(-c2nc(-c3cc4ccccc4c4ccccc34)nc(-c3cccc4oc5ccc(-c6ccc7ccccc7c6)cc5c34)n2)cc1]

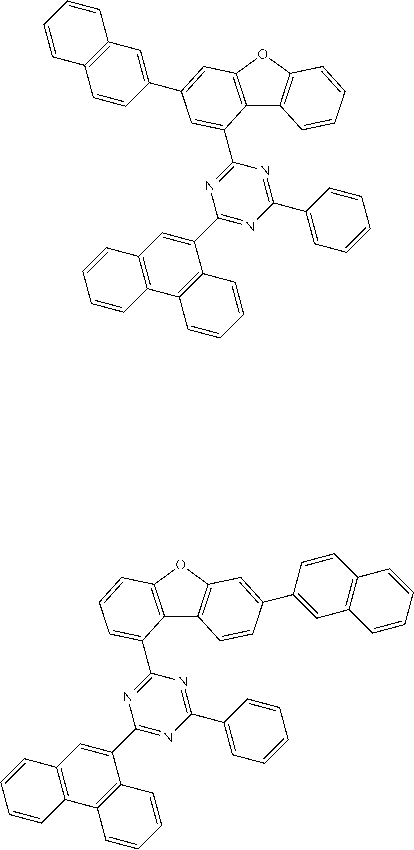 [SMILES: c1ccc(-c2nc(-c3cc4ccccc4c4ccccc34)nc(-c3cc(-c4ccc5ccccc5c4)cc4oc5ccccc5c34)n2)cc1 ; c1ccc(-c2nc(-c3cc4ccccc4c4ccccc34)nc(-c3cccc4oc5cc(-c6ccc7ccccc7c6)ccc5c34)n2)cc1]

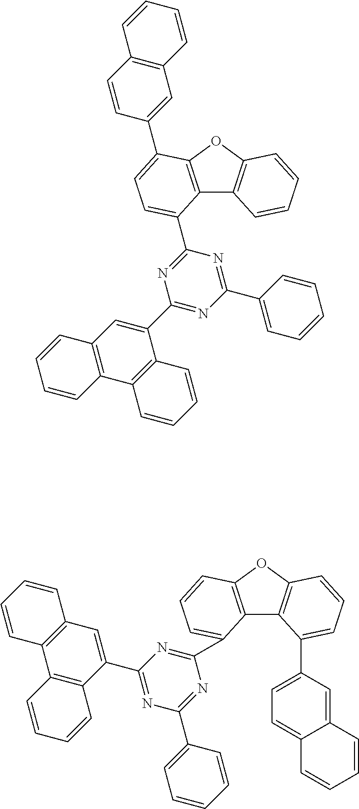 [SMILES: c1ccc(-c2nc(-c3cc4ccccc4c4ccccc34)nc(-c3ccc(-c4ccc5ccccc5c4)c4oc5ccccc5c34)n2)cc1 ; c1ccc(-c2nc(-c3cc4ccccc4c4ccccc34)nc(-c3cccc4oc5cccc(-c6ccc7ccccc7c6)c5c34)n2)cc1]

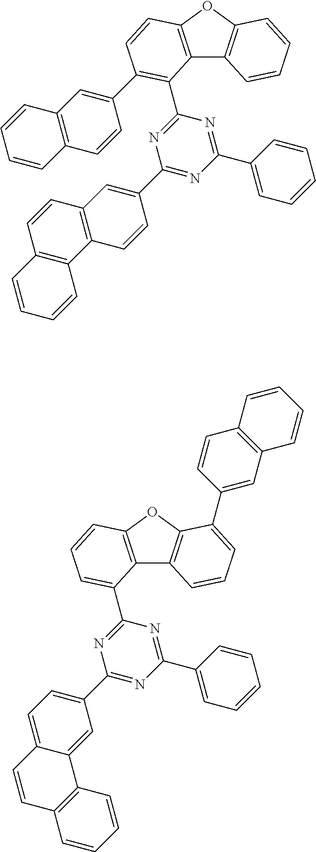 [SMILES: c1ccc(-c2nc(-c3ccc4c(ccc5ccccc54)c3)nc(-c3c(-c4ccc5ccccc5c4)ccc4oc5ccccc5c34)n2)cc1 ; c1ccc(-c2nc(-c3ccc4ccc5ccccc5c4c3)nc(-c3cccc4oc5c(-c6ccc7ccccc7c6)cccc5c34)n2)cc1]

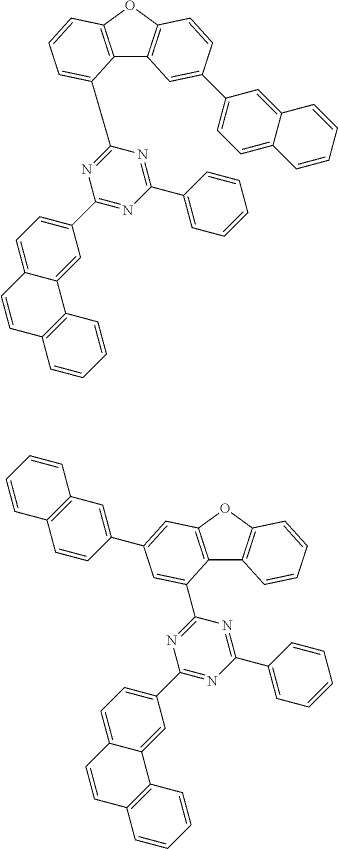 [SMILES: c1ccc(-c2nc(-c3ccc4ccc5ccccc5c4c3)nc(-c3cccc4oc5ccc(-c6ccc7ccccc7c6)cc5c34)n2)cc1 ; c1ccc(-c2nc(-c3ccc4ccc5ccccc5c4c3)nc(-c3cc(-c4ccc5ccccc5c4)cc4oc5ccccc5c34)n2)cc1]

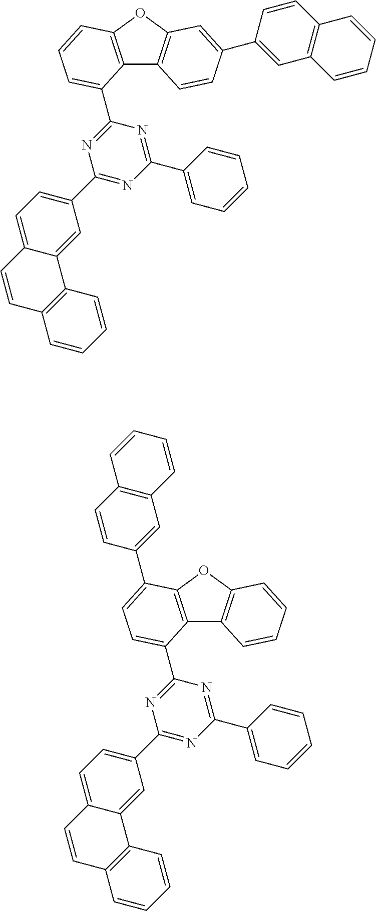 [SMILES: c1ccc(-c2nc(-c3ccc4ccc5ccccc5c4c3)nc(-c3cccc4oc5cc(-c6ccc7ccccc7c6)ccc5c34)n2)cc1 ; c1ccc(-c2nc(-c3ccc4ccc5ccccc5c4c3)nc(-c3ccc(-c4ccc5ccccc5c4)c4oc5ccccc5c34)n2)cc1]

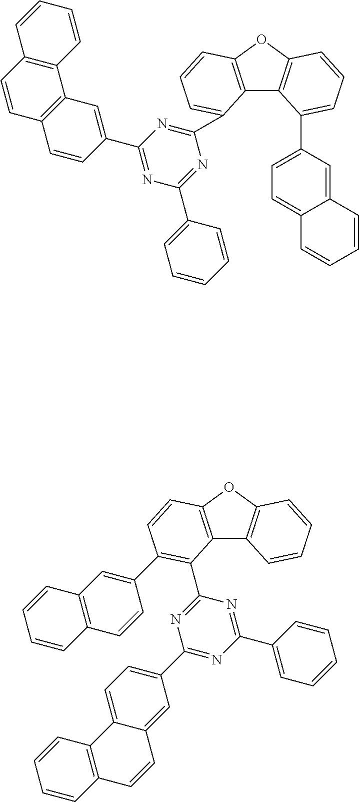 [SMILES: c1ccc(-c2nc(-c3ccc4ccc5ccccc5c4c3)nc(-c3cccc4oc5cccc(-c6ccc7ccccc7c6)c5c34)n2)cc1 ; c1ccc(-c2nc(-c3ccc4c(ccc5ccccc54)c3)nc(-c3c(-c4ccc5ccccc5c4)ccc4oc5ccccc5c34)n2)cc1]

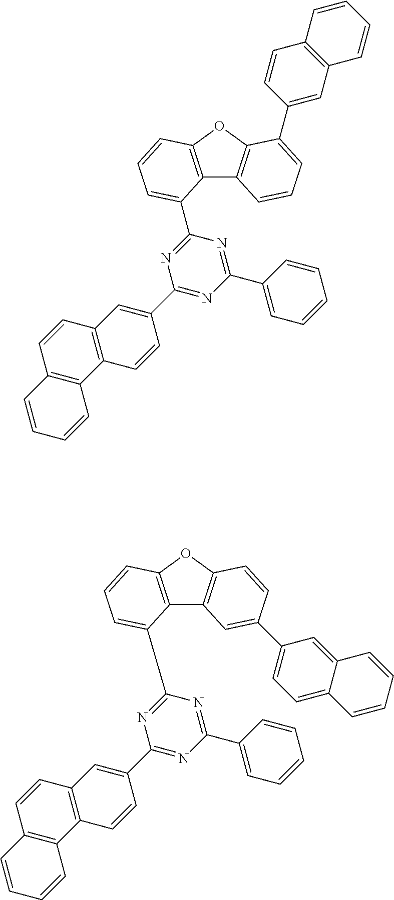 [SMILES: c1ccc(-c2nc(-c3ccc4c(ccc5ccccc54)c3)nc(-c3cccc4oc5c(-c6ccc7ccccc7c6)cccc5c34)n2)cc1 ; c1ccc(-c2nc(-c3ccc4c(ccc5ccccc54)c3)nc(-c3cccc4oc5ccc(-c6ccc7ccccc7c6)cc5c34)n2)cc1]

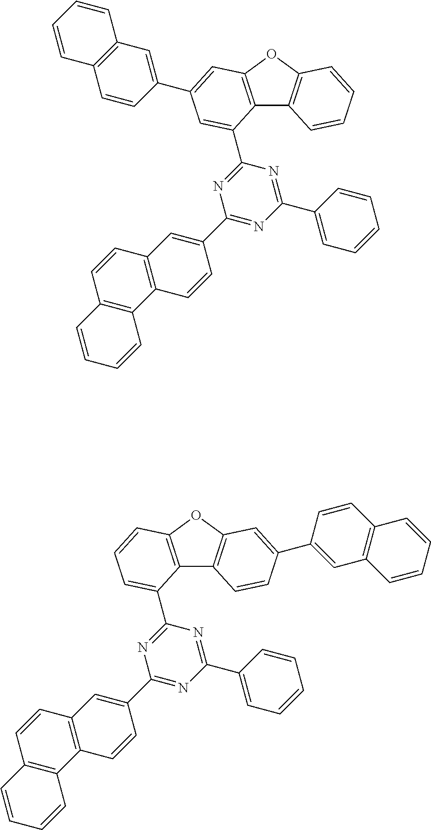 [SMILES: c1ccc(-c2nc(-c3ccc4c(ccc5ccccc54)c3)nc(-c3cc(-c4ccc5ccccc5c4)cc4oc5ccccc5c34)n2)cc1 ; c1ccc(-c2nc(-c3ccc4c(ccc5ccccc54)c3)nc(-c3cccc4oc5cc(-c6ccc7ccccc7c6)ccc5c34)n2)cc1]

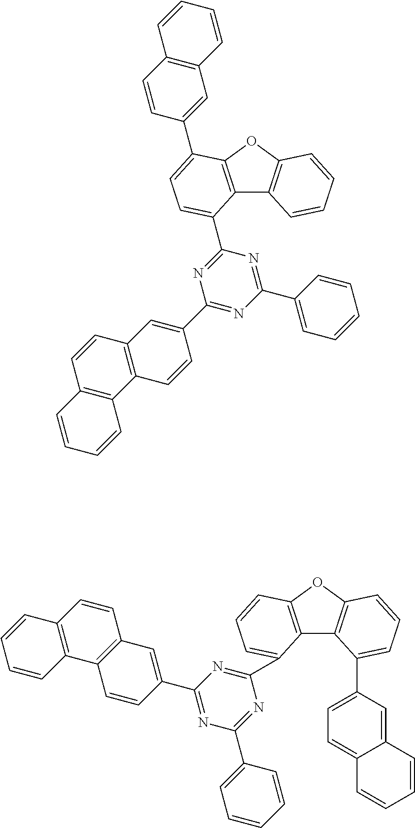 [SMILES: c1ccc(-c2nc(-c3ccc4c(ccc5ccccc54)c3)nc(-c3ccc(-c4ccc5ccccc5c4)c4oc5ccccc5c34)n2)cc1 ; c1ccc(-c2nc(-c3ccc4c(ccc5ccccc54)c3)nc(-c3cccc4oc5cccc(-c6ccc7ccccc7c6)c5c34)n2)cc1]

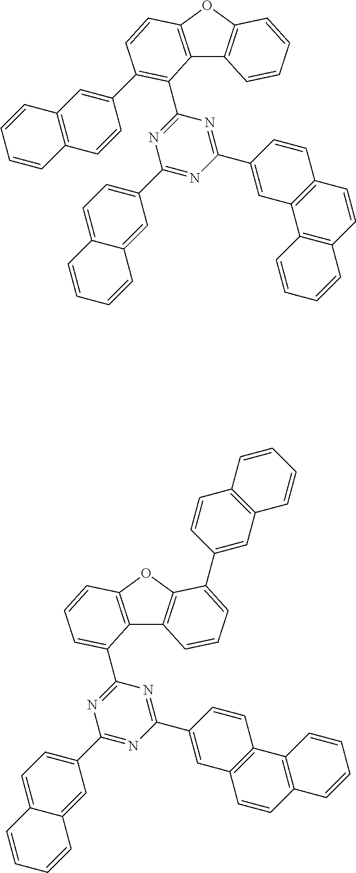 [SMILES: c1ccc2cc(-c3nc(-c4ccc5ccc6ccccc6c5c4)nc(-c4c(-c5ccc6ccccc6c5)ccc5oc6ccccc6c45)n3)ccc2c1 ; c1ccc2cc(-c3nc(-c4ccc5c(ccc6ccccc65)c4)nc(-c4cccc5oc6c(-c7ccc8ccccc8c7)cccc6c45)n3)ccc2c1]

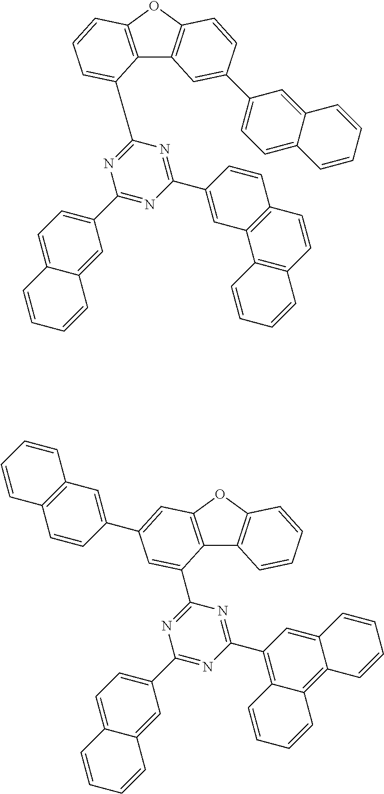 [SMILES: c1ccc2cc(-c3ccc4oc5cccc(-c6nc(-c7ccc8ccccc8c7)nc(-c7ccc8ccc9ccccc9c8c7)n6)c5c4c3)ccc2c1 ; c1ccc2cc(-c3cc(-c4nc(-c5ccc6ccccc6c5)nc(-c5cc6ccccc6c6ccccc56)n4)c4c(c3)oc3ccccc34)ccc2c1]

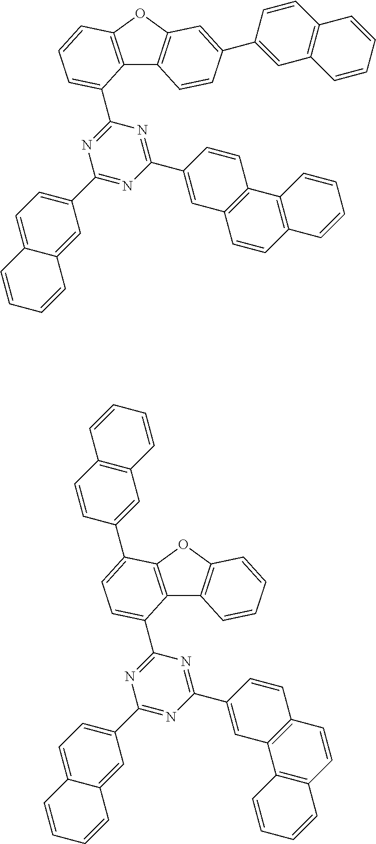 [SMILES: c1ccc2cc(-c3ccc4c(c3)oc3cccc(-c5nc(-c6ccc7ccccc7c6)nc(-c6ccc7c(ccc8ccccc87)c6)n5)c34)ccc2c1 ; c1ccc2cc(-c3nc(-c4ccc5ccc6ccccc6c5c4)nc(-c4ccc(-c5ccc6ccccc6c5)c5oc6ccccc6c45)n3)ccc2c1]

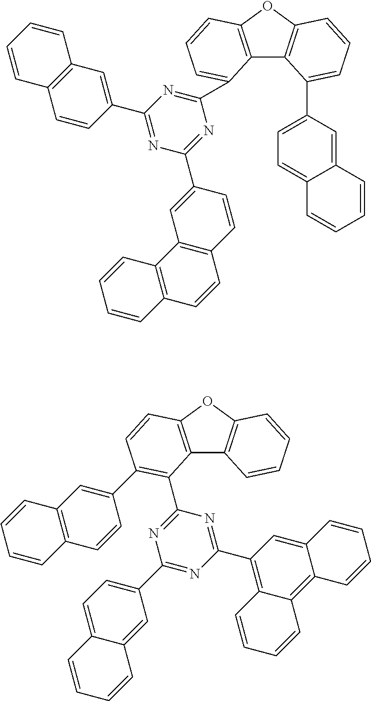 [SMILES: c1ccc2cc(-c3nc(-c4ccc5ccc6ccccc6c5c4)nc(-c4cccc5oc6cccc(-c7ccc8ccccc8c7)c6c45)n3)ccc2c1 ; c1ccc2cc(-c3nc(-c4cc5ccccc5c5ccccc45)nc(-c4c(-c5ccc6ccccc6c5)ccc5oc6ccccc6c45)n3)ccc2c1]

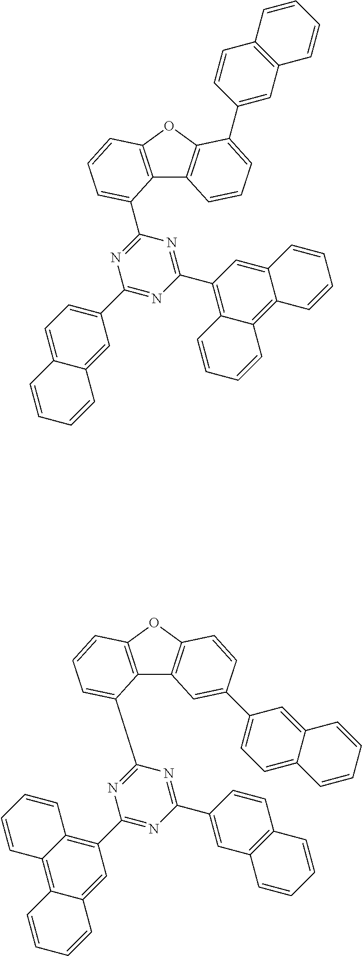 [SMILES: c1ccc2cc(-c3nc(-c4cc5ccccc5c5ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cc8ccccc8c8ccccc78)n6)c5c4c3)ccc2c1]

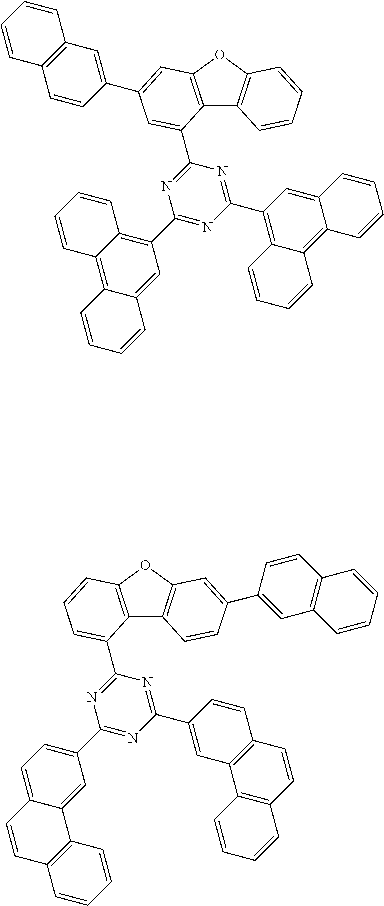 [SMILES: c1ccc2cc(-c3cc(-c4nc(-c5cc6ccccc6c6ccccc56)nc(-c5cc6ccccc6c6ccccc56)n4)c4c(c3)oc3ccccc34)ccc2c1 ; c1ccc2cc(-c3ccc4c(c3)oc3cccc(-c5nc(-c6ccc7ccc8ccccc8c7c6)nc(-c6ccc7ccc8ccccc8c7c6)n5)c34)ccc2c1]

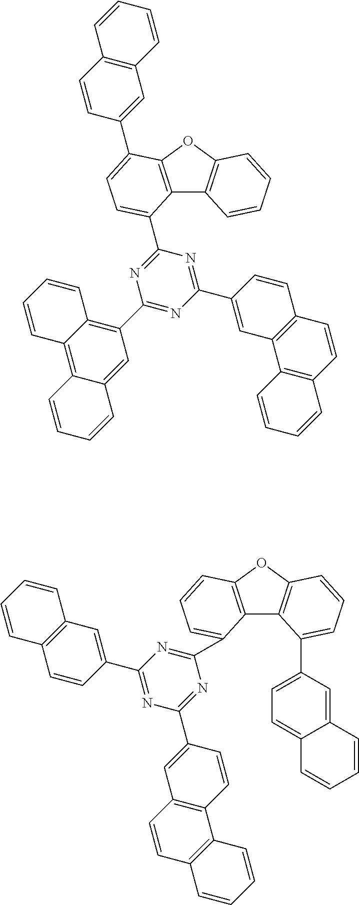 [SMILES: c1ccc2cc(-c3ccc(-c4nc(-c5ccc6ccc7ccccc7c6c5)nc(-c5cc6ccccc6c6ccccc56)n4)c4c3oc3ccccc34)ccc2c1 ; c1ccc2cc(-c3nc(-c4ccc5c(ccc6ccccc65)c4)nc(-c4cccc5oc6cccc(-c7ccc8ccccc8c7)c6c45)n3)ccc2c1]

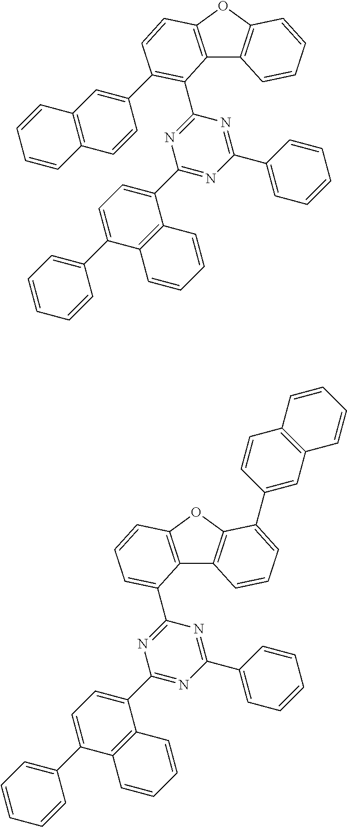 [SMILES: c1ccc(-c2nc(-c3ccc(-c4ccccc4)c4ccccc34)nc(-c3c(-c4ccc5ccccc5c4)ccc4oc5ccccc5c34)n2)cc1 ; c1ccc(-c2nc(-c3ccc(-c4ccccc4)c4ccccc34)nc(-c3cccc4oc5c(-c6ccc7ccccc7c6)cccc5c34)n2)cc1]

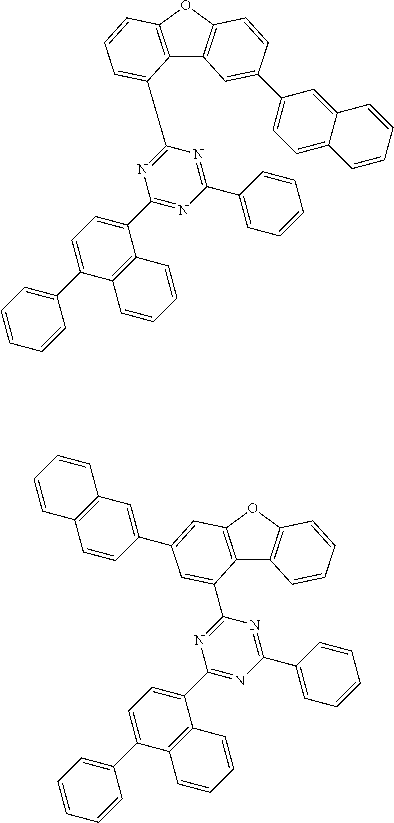 [SMILES: c1ccc(-c2nc(-c3ccc(-c4ccccc4)c4ccccc34)nc(-c3cccc4oc5ccc(-c6ccc7ccccc7c6)cc5c34)n2)cc1 ; c1ccc(-c2nc(-c3ccc(-c4ccccc4)c4ccccc34)nc(-c3cc(-c4ccc5ccccc5c4)cc4oc5ccccc5c34)n2)cc1]

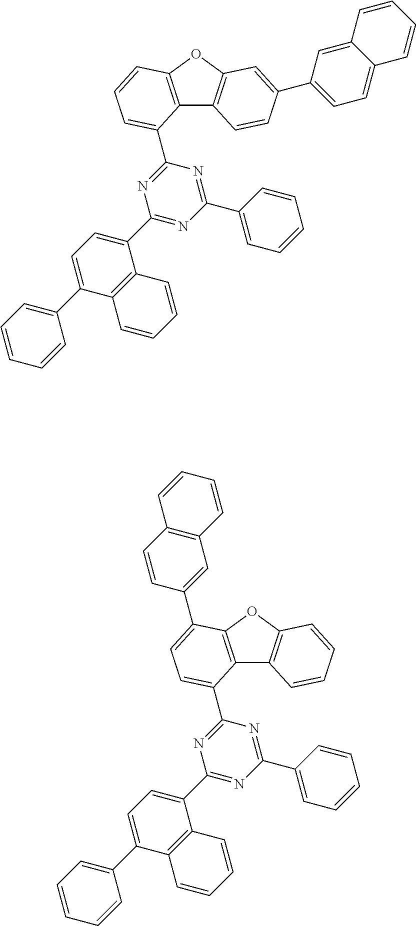 [SMILES: c1ccc(-c2nc(-c3ccc(-c4ccccc4)c4ccccc34)nc(-c3cccc4oc5cc(-c6ccc7ccccc7c6)ccc5c34)n2)cc1 ; c1ccc(-c2nc(-c3ccc(-c4ccccc4)c4ccccc34)nc(-c3ccc(-c4ccc5ccccc5c4)c4oc5ccccc5c34)n2)cc1]

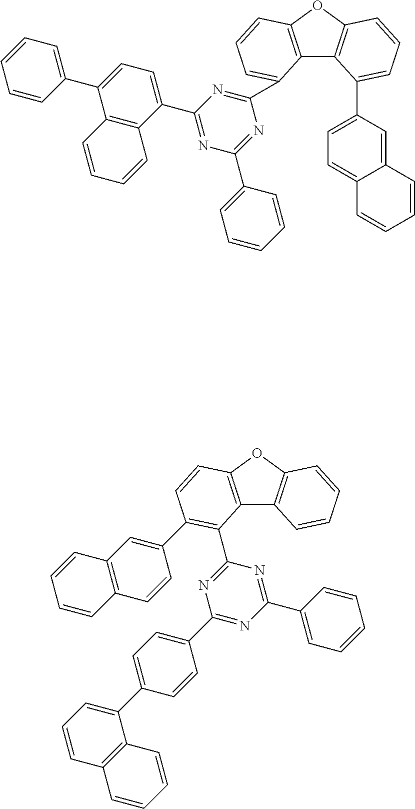 [SMILES: c1ccc(-c2nc(-c3ccc(-c4ccccc4)c4ccccc34)nc(-c3cccc4oc5cccc(-c6ccc7ccccc7c6)c5c34)n2)cc1 ; c1ccc(-c2nc(-c3ccc(-c4cccc5ccccc45)cc3)nc(-c3c(-c4ccc5ccccc5c4)ccc4oc5ccccc5c34)n2)cc1]

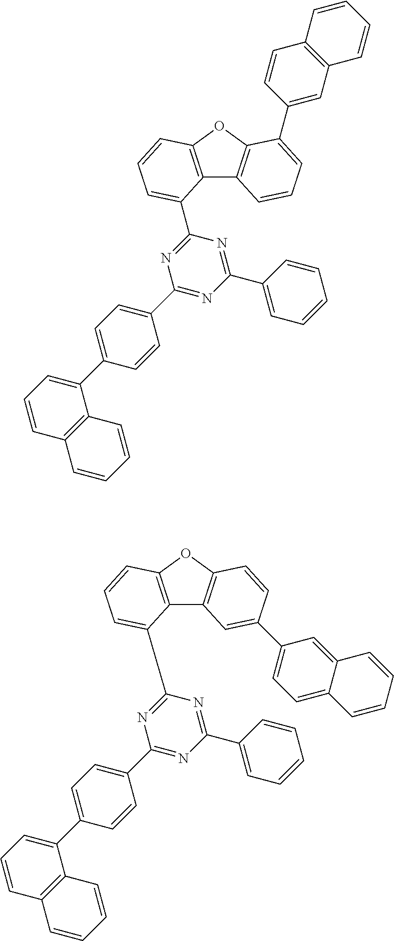 [SMILES: c1ccc(-c2nc(-c3ccc(-c4cccc5ccccc45)cc3)nc(-c3cccc4oc5c(-c6ccc7ccccc7c6)cccc5c34)n2)cc1 ; c1ccc(-c2nc(-c3ccc(-c4cccc5ccccc45)cc3)nc(-c3cccc4oc5ccc(-c6ccc7ccccc7c6)cc5c34)n2)cc1]

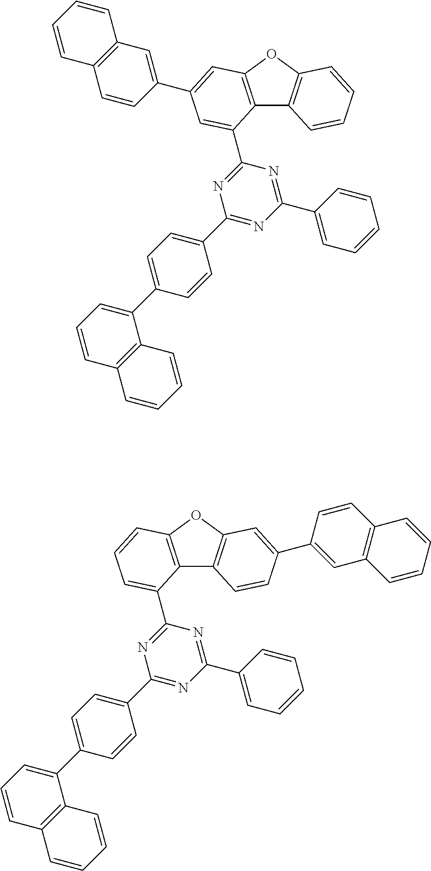 [SMILES: c1ccc(-c2nc(-c3ccc(-c4cccc5ccccc45)cc3)nc(-c3cc(-c4ccc5ccccc5c4)cc4oc5ccccc5c34)n2)cc1 ; c1ccc(-c2nc(-c3ccc(-c4cccc5ccccc45)cc3)nc(-c3cccc4oc5cc(-c6ccc7ccccc7c6)ccc5c34)n2)cc1]

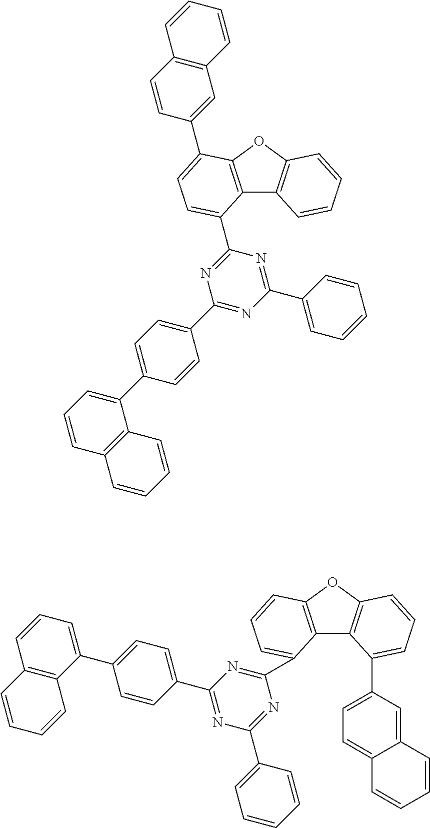 [SMILES: c1ccc(-c2nc(-c3ccc(-c4cccc5ccccc45)cc3)nc(-c3ccc(-c4ccc5ccccc5c4)c4oc5ccccc5c34)n2)cc1 ; c1ccc(-c2nc(-c3ccc(-c4cccc5ccccc45)cc3)nc(-c3cccc4oc5cccc(-c6ccc7ccccc7c6)c5c34)n2)cc1]

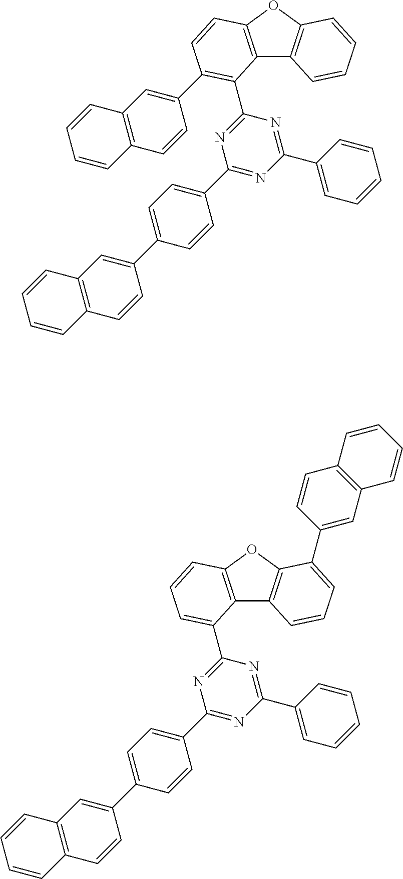 [SMILES: c1ccc(-c2nc(-c3ccc(-c4ccc5ccccc5c4)cc3)nc(-c3c(-c4ccc5ccccc5c4)ccc4oc5ccccc5c34)n2)cc1 ; c1ccc(-c2nc(-c3ccc(-c4ccc5ccccc5c4)cc3)nc(-c3cccc4oc5c(-c6ccc7ccccc7c6)cccc5c34)n2)cc1]

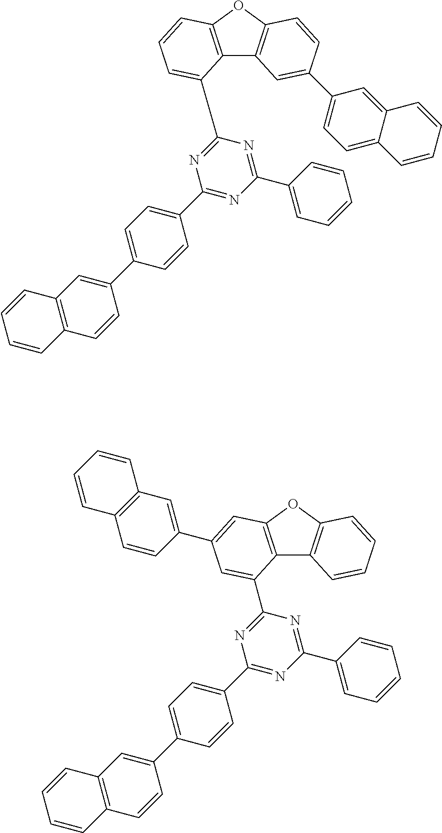 [SMILES: c1ccc(-c2nc(-c3ccc(-c4ccc5ccccc5c4)cc3)nc(-c3cccc4oc5ccc(-c6ccc7ccccc7c6)cc5c34)n2)cc1 ; c1ccc(-c2nc(-c3ccc(-c4ccc5ccccc5c4)cc3)nc(-c3cc(-c4ccc5ccccc5c4)cc4oc5ccccc5c34)n2)cc1]

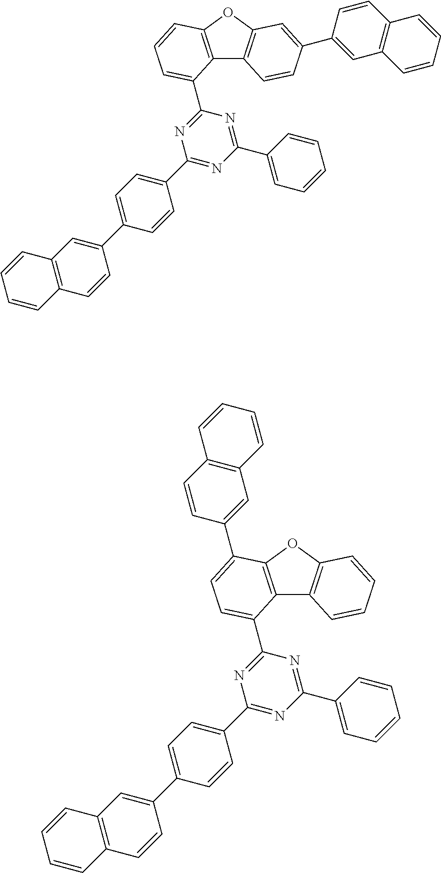 [SMILES: c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1]

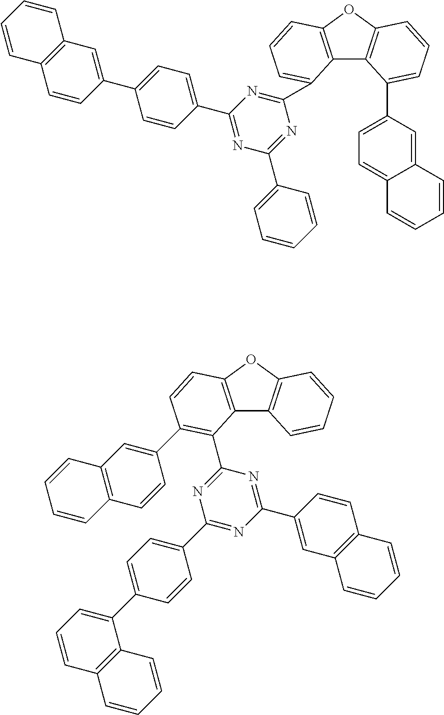 [SMILES: c1ccc(-c2nc(-c3ccc(-c4ccc5ccccc5c4)cc3)nc(-c3cccc4oc5cccc(-c6ccc7ccccc7c6)c5c34)n2)cc1 ; c1ccc2cc(-c3nc(-c4ccc(-c5cccc6ccccc56)cc4)nc(-c4c(-c5ccc6ccccc6c5)ccc5oc6ccccc6c45)n3)ccc2c1]

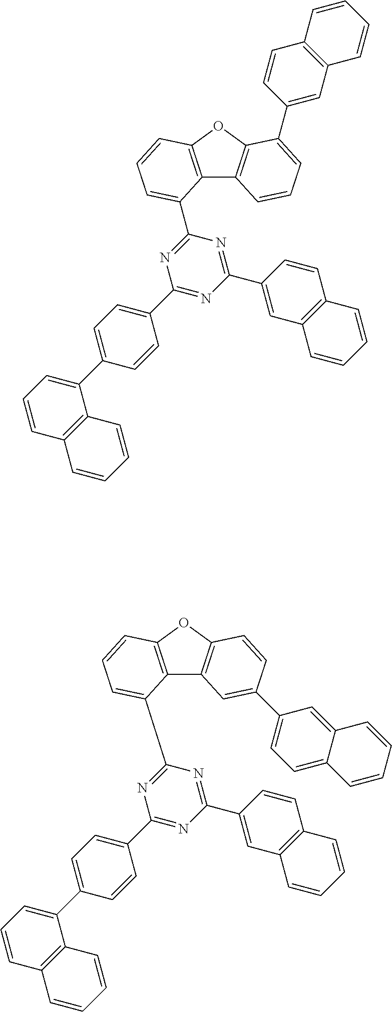 [SMILES: c1ccc2cc(-c3nc(-c4ccc(-c5cccc6ccccc56)cc4)nc(-c4cccc5oc6c(-c7ccc8ccccc8c7)cccc6c45)n3)ccc2c1 ; c1ccc2cc(-c3ccc4oc5cccc(-c6nc(-c7ccc(-c8cccc9ccccc89)cc7)nc(-c7ccc8ccccc8c7)n6)c5c4c3)ccc2c1]

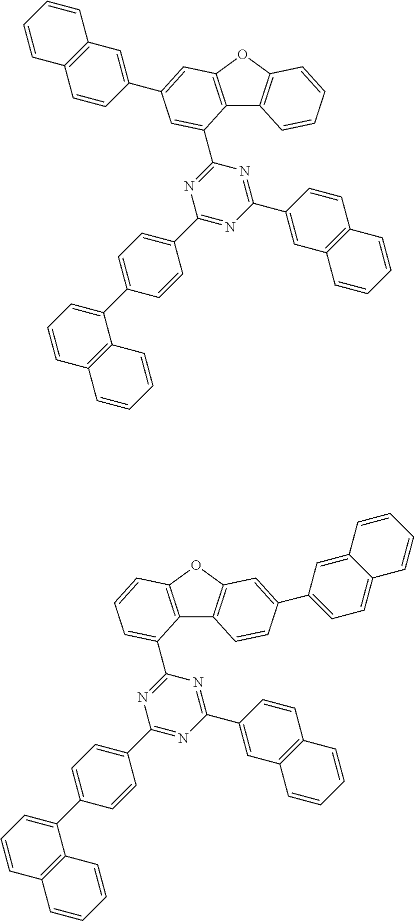 [SMILES: c1ccc2cc(-c3cc(-c4nc(-c5ccc(-c6cccc7ccccc67)cc5)nc(-c5ccc6ccccc6c5)n4)c4c(c3)oc3ccccc34)ccc2c1 ; c1ccc2cc(-c3ccc4c(c3)oc3cccc(-c5nc(-c6ccc(-c7cccc8ccccc78)cc6)nc(-c6ccc7ccccc7c6)n5)c34)ccc2c1]

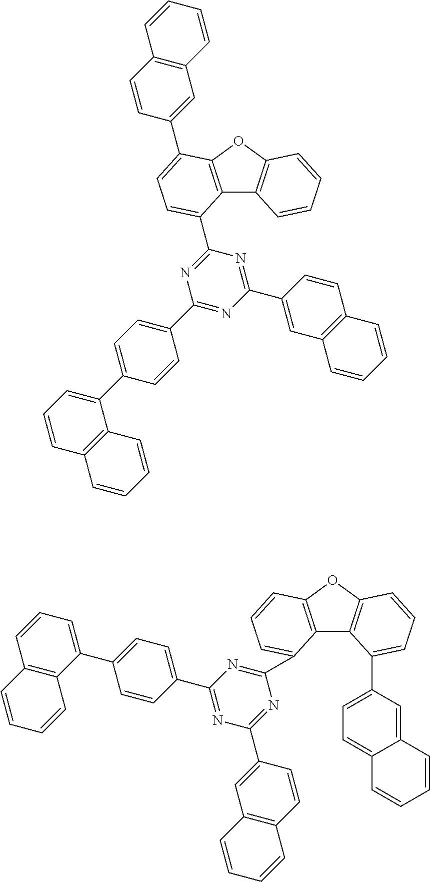 [SMILES: c1ccc2cc(-c3nc(-c4ccc(-c5cccc6ccccc56)cc4)nc(-c4ccc(-c5ccc6ccccc6c5)c5oc6ccccc6c45)n3)ccc2c1 ; c1ccc2cc(-c3nc(-c4ccc(-c5cccc6ccccc56)cc4)nc(-c4cccc5oc6cccc(-c7ccc8ccccc8c7)c6c45)n3)ccc2c1]

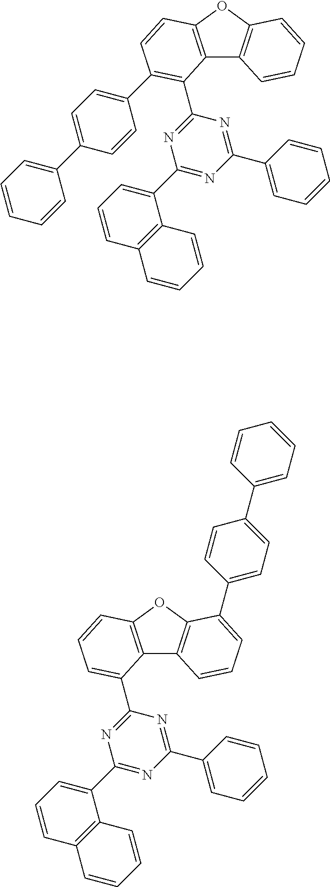 [SMILES: c1ccc(-c2ccc(-c3ccc4oc5ccccc5c4c3-c3nc(-c4ccccc4)nc(-c4cccc5ccccc45)n3)cc2)cc1 ; c1ccc(-c2ccc(-c3cccc4c3oc3cccc(-c5nc(-c6ccccc6)nc(-c6cccc7ccccc67)n5)c34)cc2)cc1]

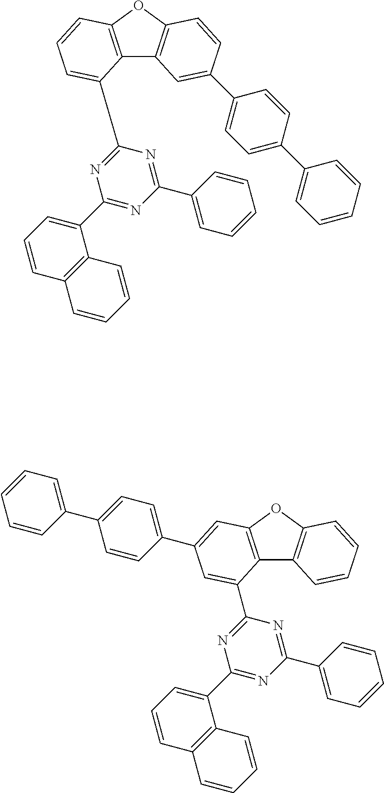 [SMILES: c1ccc(-c2ccc(-c3ccc4oc5cccc(-c6nc(-c7ccccc7)nc(-c7cccc8ccccc78)n6)c5c4c3)cc2)cc1 ; c1ccc(-c2ccc(-c3cc(-c4nc(-c5ccccc5)nc(-c5cccc6ccccc56)n4)c4c(c3)oc3ccccc34)cc2)cc1]

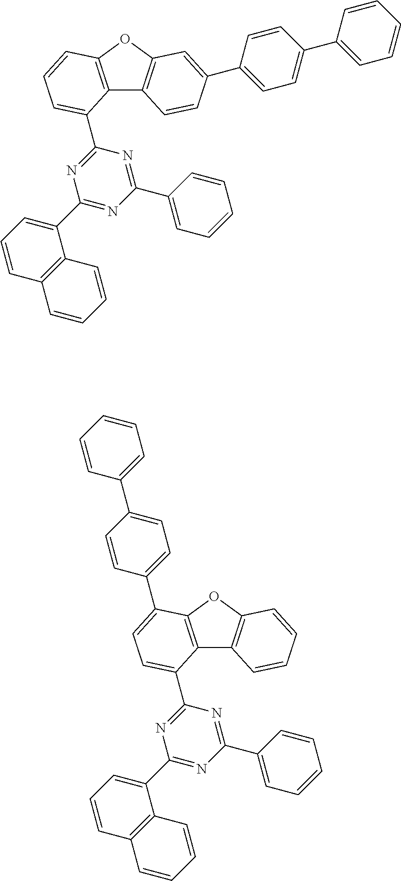 [SMILES: c1ccc(-c2ccc(-c3ccc4c(c3)oc3cccc(-c5nc(-c6ccccc6)nc(-c6cccc7ccccc67)n5)c34)cc2)cc1 ; c1ccc(-c2ccc(-c3ccc(-c4nc(-c5ccccc5)nc(-c5cccc6ccccc56)n4)c4c3oc3ccccc34)cc2)cc1]

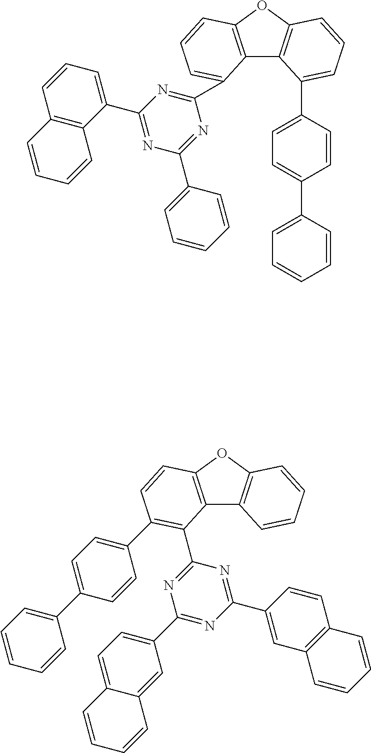 [SMILES: c1ccc(-c2ccc(-c3cccc4oc5cccc(-c6nc(-c7ccccc7)nc(-c7cccc8ccccc78)n6)c5c34)cc2)cc1 ; c1ccc(-c2ccc(-c3ccc4oc5ccccc5c4c3-c3nc(-c4ccc5ccccc5c4)nc(-c4ccc5ccccc5c4)n3)cc2)cc1]

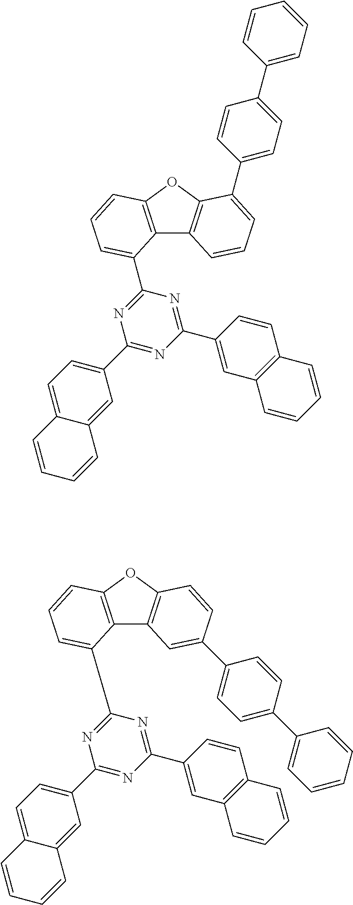 [SMILES: c1ccc(-c2ccc(-c3cccc4c3oc3cccc(-c5nc(-c6ccc7ccccc7c6)nc(-c6ccc7ccccc7c6)n5)c34)cc2)cc1 ; c1ccc(-c2ccc(-c3ccc4oc5cccc(-c6nc(-c7ccc8ccccc8c7)nc(-c7ccc8ccccc8c7)n6)c5c4c3)cc2)cc1]

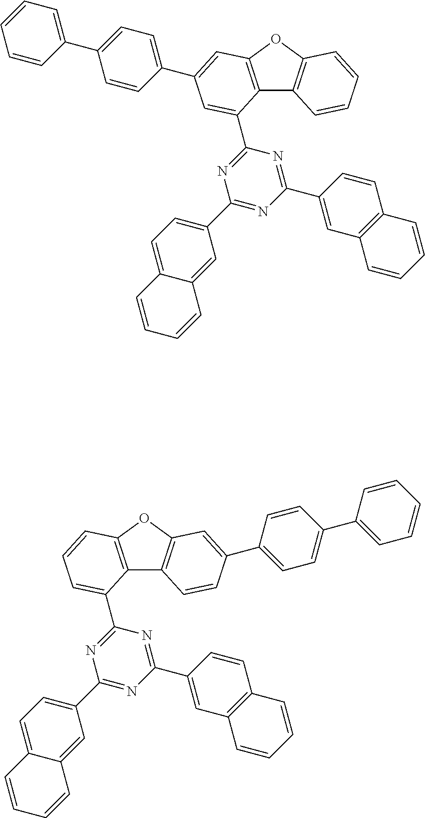 [SMILES: c1ccc(-c2ccc(-c3cc(-c4nc(-c5ccc6ccccc6c5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7ccccc7c6)n5)c34)cc2)cc1]

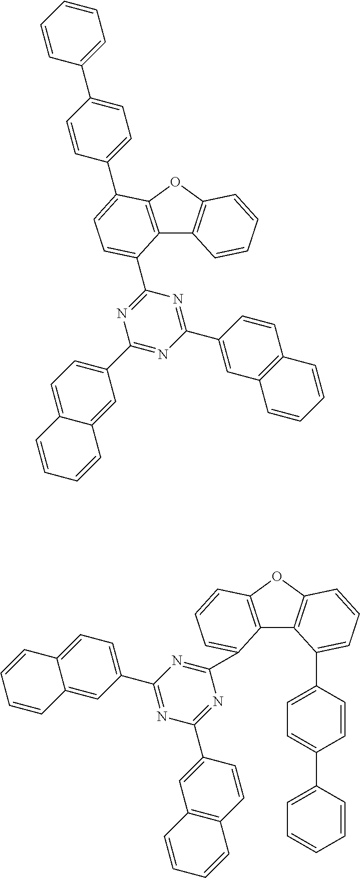 [SMILES: c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1]

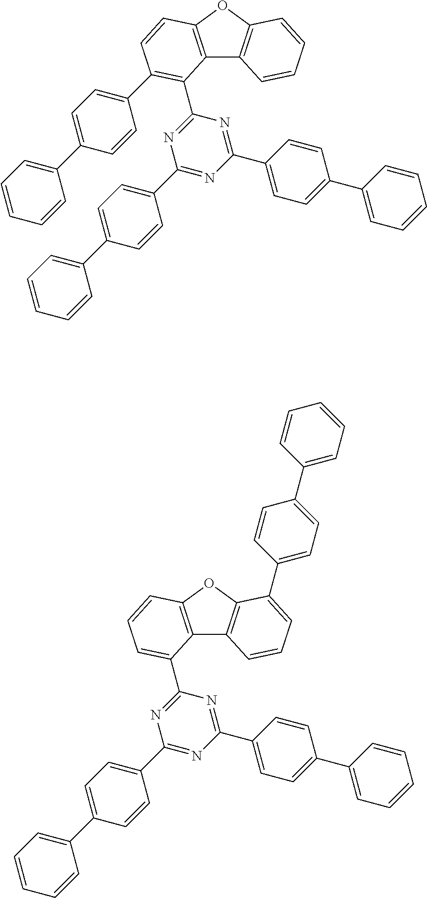 [SMILES: c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc(-c8ccccc8)cc7)cccc6c45)n3)cc2)cc1]

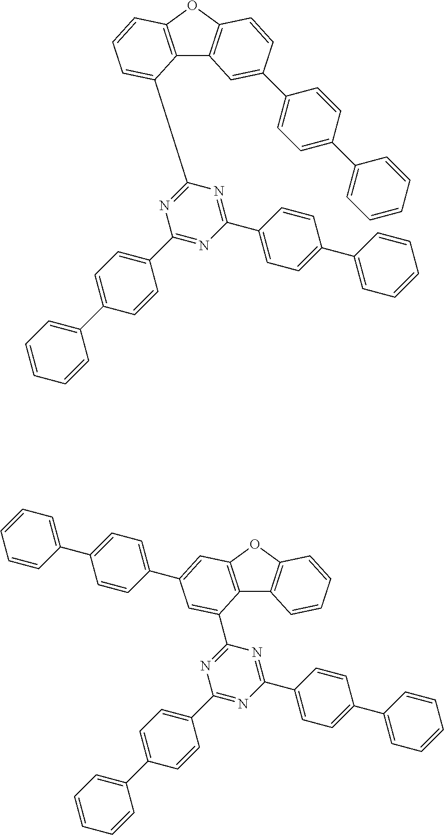 [SMILES: c1ccc(-c2ccc(-c3ccc4oc5cccc(-c6nc(-c7ccc(-c8ccccc8)cc7)nc(-c7ccc(-c8ccccc8)cc7)n6)c5c4c3)cc2)cc1 ; c1ccc(-c2ccc(-c3cc(-c4nc(-c5ccc(-c6ccccc6)cc5)nc(-c5ccc(-c6ccccc6)cc5)n4)c4c(c3)oc3ccccc34)cc2)cc1]

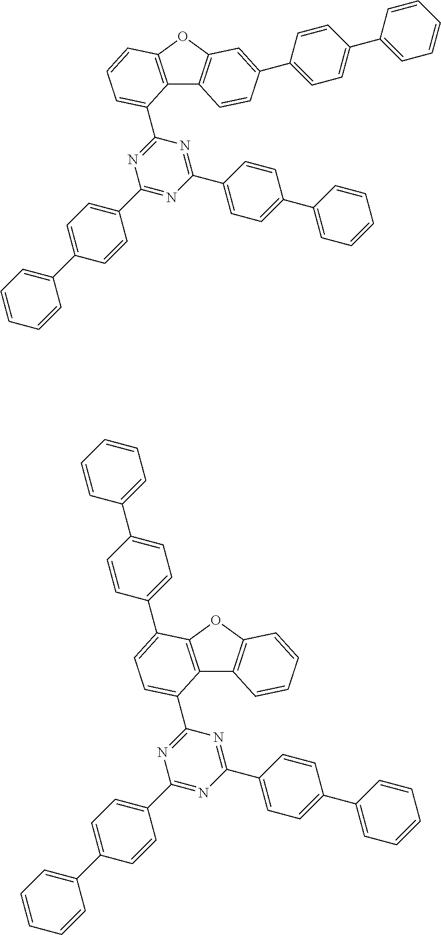 [SMILES: c1ccc(-c2ccc(-c3ccc4c(c3)oc3cccc(-c5nc(-c6ccc(-c7ccccc7)cc6)nc(-c6ccc(-c7ccccc7)cc6)n5)c34)cc2)cc1 ; c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1]

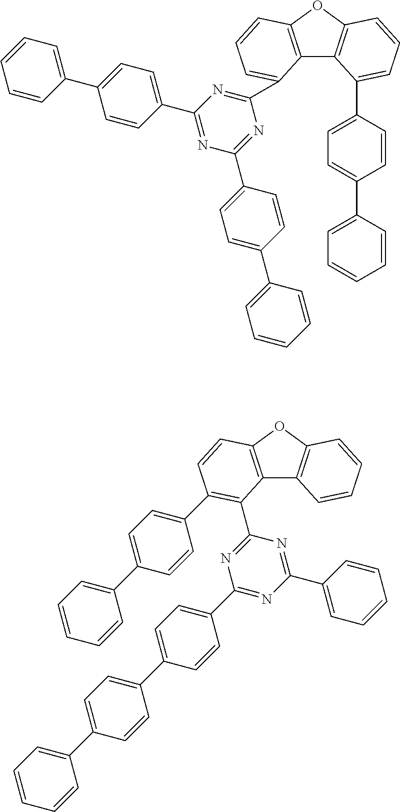 [SMILES: c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1]

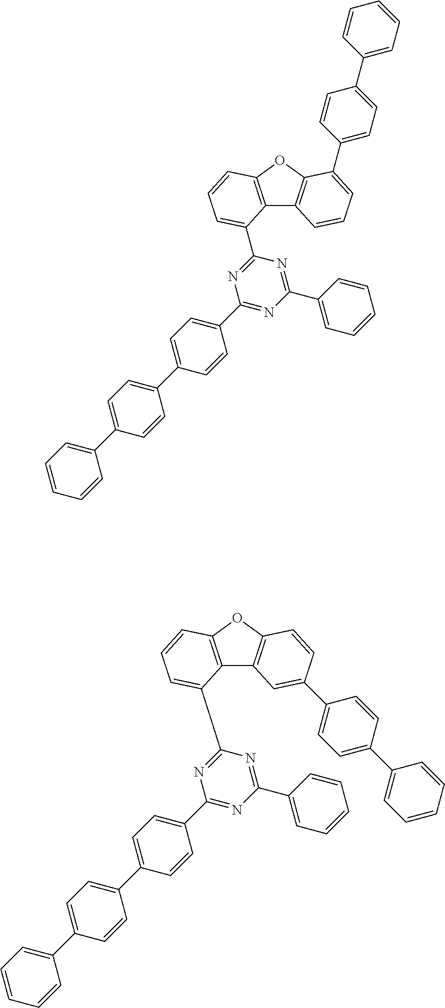 [SMILES: c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1]

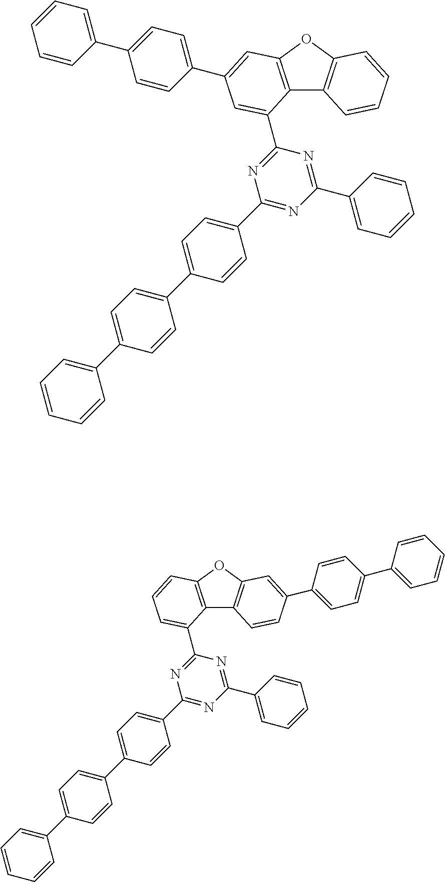 [SMILES: c1ccc(-c2ccc(-c3ccc(-c4nc(-c5ccccc5)nc(-c5cc(-c6ccc(-c7ccccc7)cc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc(-c9ccccc9)cc8)ccc7c56)n4)cc3)cc2)cc1]

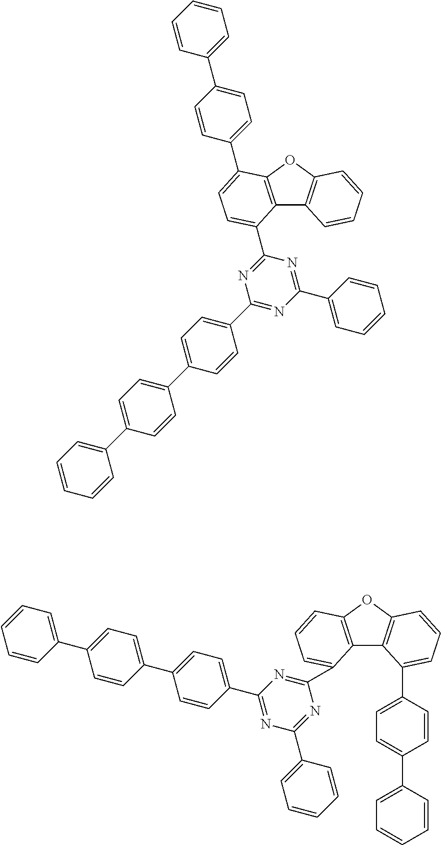 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

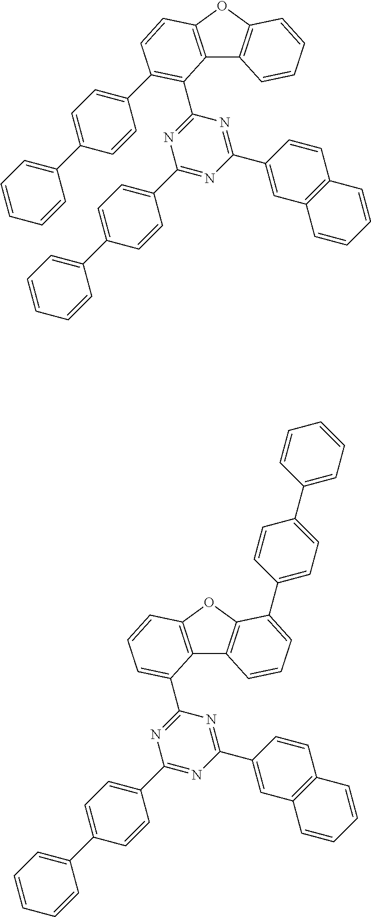 [SMILES: c1ccc(-c2ccc(-c3nc(-c4ccc5ccccc5c4)nc(-c4c(-c5ccc(-c6ccccc6)cc5)ccc5oc6ccccc6c45)n3)cc2)cc1 ; c1ccc(-c2ccc(-c3nc(-c4ccc5ccccc5c4)nc(-c4cccc5oc6c(-c7ccc(-c8ccccc8)cc7)cccc6c45)n3)cc2)cc1]

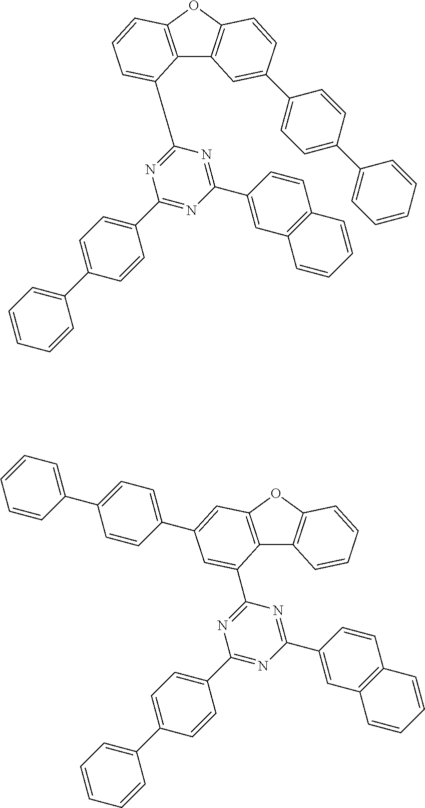 [SMILES: c1ccc(-c2ccc(-c3ccc4oc5cccc(-c6nc(-c7ccc(-c8ccccc8)cc7)nc(-c7ccc8ccccc8c7)n6)c5c4c3)cc2)cc1 ; c1ccc(-c2ccc(-c3cc(-c4nc(-c5ccc(-c6ccccc6)cc5)nc(-c5ccc6ccccc6c5)n4)c4c(c3)oc3ccccc34)cc2)cc1]

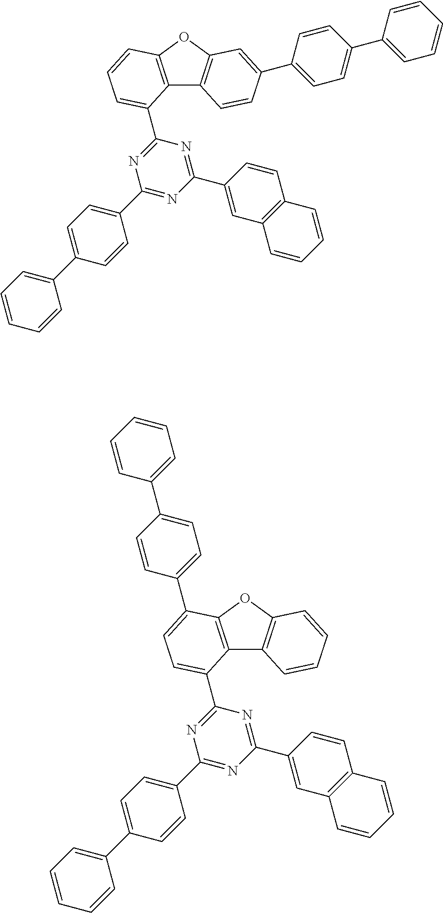 [SMILES: c1ccc(-c2ccc(-c3ccc4c(c3)oc3cccc(-c5nc(-c6ccc(-c7ccccc7)cc6)nc(-c6ccc7ccccc7c6)n5)c34)cc2)cc1 ; c1ccc(-c2ccc(-c3nc(-c4ccc5ccccc5c4)nc(-c4ccc(-c5ccc(-c6ccccc6)cc5)c5oc6ccccc6c45)n3)cc2)cc1]

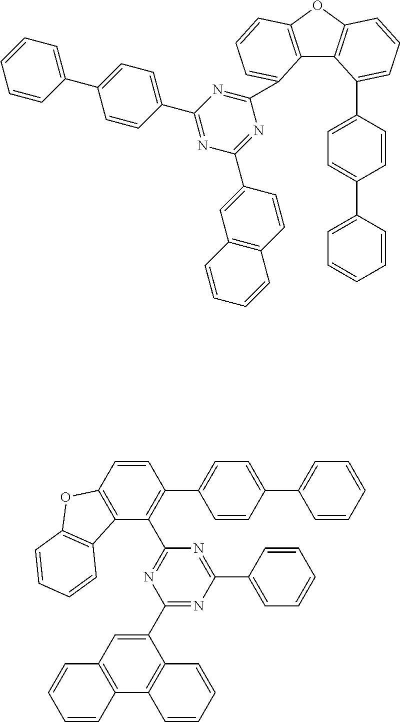 [SMILES: c1ccc(-c2ccc(-c3nc(-c4ccc5ccccc5c4)nc(-c4cccc5oc6cccc(-c7ccc(-c8ccccc8)cc7)c6c45)n3)cc2)cc1 ; c1ccc(-c2ccc(-c3ccc4oc5ccccc5c4c3-c3nc(-c4ccccc4)nc(-c4cc5ccccc5c5ccccc45)n3)cc2)cc1]

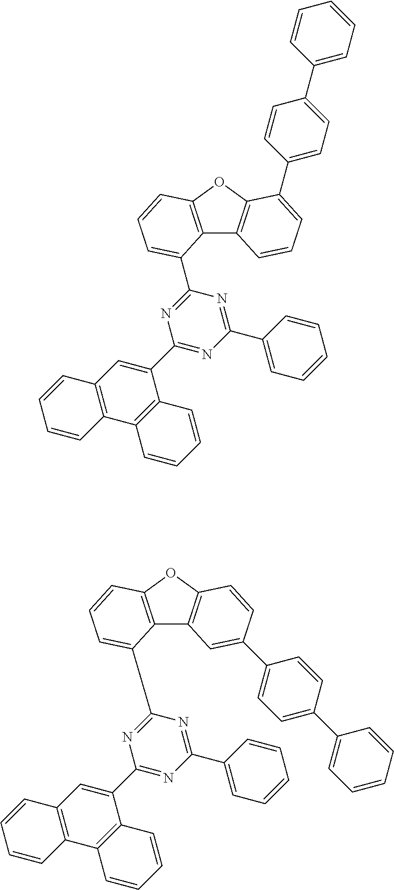 [SMILES: c1ccc(-c2ccc(-c3cccc4c3oc3cccc(-c5nc(-c6ccccc6)nc(-c6cc7ccccc7c7ccccc67)n5)c34)cc2)cc1 ; c1ccc(-c2ccc(-c3ccc4oc5cccc(-c6nc(-c7ccccc7)nc(-c7cc8ccccc8c8ccccc78)n6)c5c4c3)cc2)cc1]

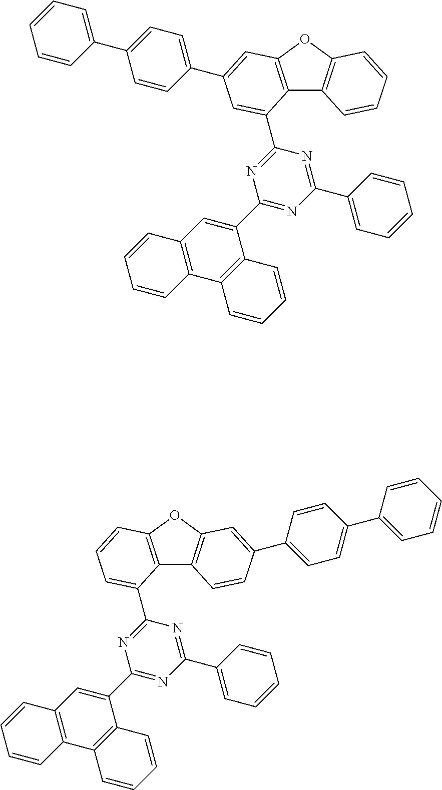 [SMILES: c1ccc(-c2ccc(-c3cc(-c4nc(-c5ccccc5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6cc7ccccc7c7ccccc67)n5)c34)cc2)cc1]

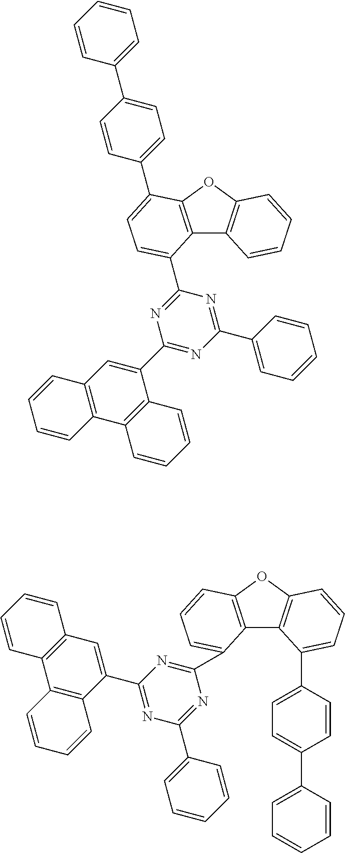 [SMILES: c1ccc(-c2ccc(-c3ccc(-c4nc(-c5ccccc5)nc(-c5cc6ccccc6c6ccccc56)n4)c4c3oc3ccccc34)cc2)cc1 ; c1ccc(-c2ccc(-c3cccc4oc5cccc(-c6nc(-c7ccccc7)nc(-c7cc8ccccc8c8ccccc78)n6)c5c34)cc2)cc1]

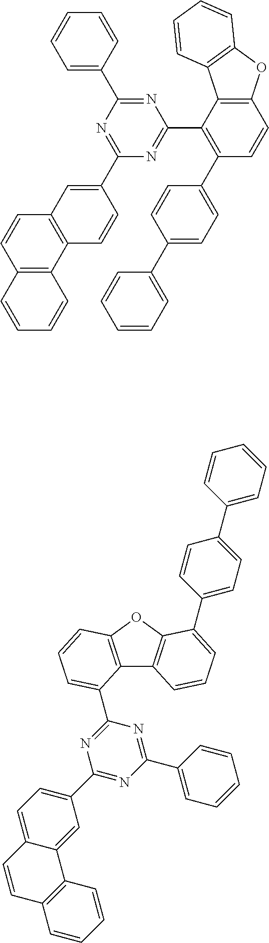 [SMILES: c1ccc(-c2ccc(-c3ccc4oc5ccccc5c4c3-c3nc(-c4ccccc4)nc(-c4ccc5c(ccc6ccccc65)c4)n3)cc2)cc1 ; c1ccc(-c2ccc(-c3cccc4c3oc3cccc(-c5nc(-c6ccccc6)nc(-c6ccc7ccc8ccccc8c7c6)n5)c34)cc2)cc1]

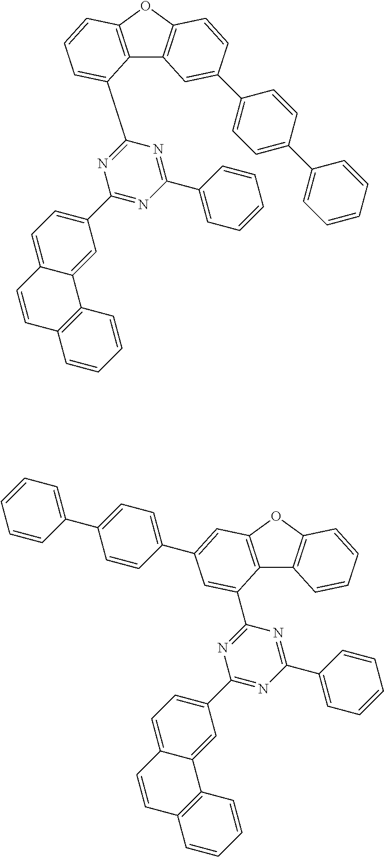 [SMILES: c1ccc(-c2ccc(-c3ccc4oc5cccc(-c6nc(-c7ccccc7)nc(-c7ccc8ccc9ccccc9c8c7)n6)c5c4c3)cc2)cc1 ; c1ccc(-c2ccc(-c3cc(-c4nc(-c5ccccc5)nc(-c5ccc6ccc7ccccc7c6c5)n4)c4c(c3)oc3ccccc34)cc2)cc1]

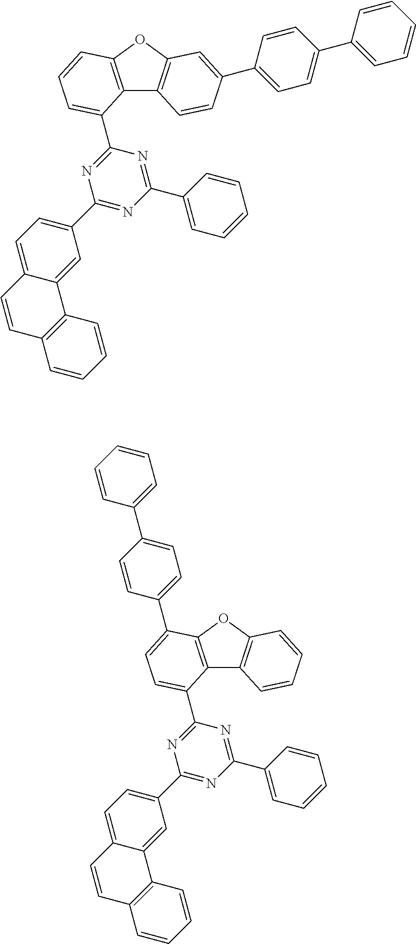 [SMILES: c1ccc(-c2ccc(-c3ccc4c(c3)oc3cccc(-c5nc(-c6ccccc6)nc(-c6ccc7ccc8ccccc8c7c6)n5)c34)cc2)cc1 ; c1ccc(-c2ccc(-c3ccc(-c4nc(-c5ccccc5)nc(-c5ccc6ccc7ccccc7c6c5)n4)c4c3oc3ccccc34)cc2)cc1]

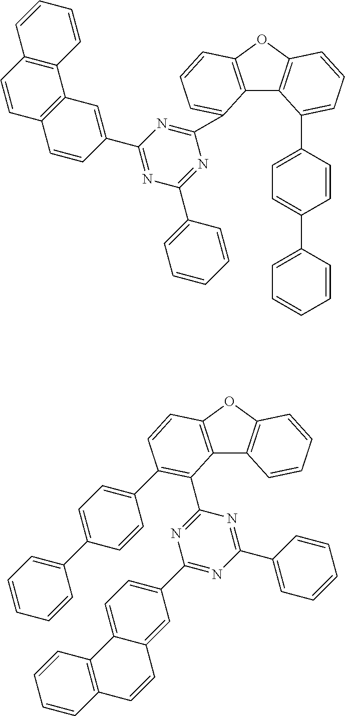 [SMILES: c1ccc(-c2ccc(-c3cccc4oc5cccc(-c6nc(-c7ccccc7)nc(-c7ccc8ccc9ccccc9c8c7)n6)c5c34)cc2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1]

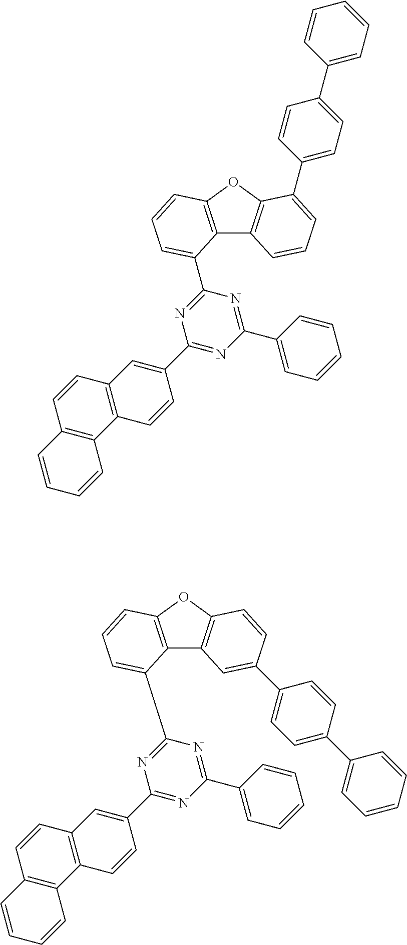 [SMILES: c1ccc(-c2ccc(-c3cccc4c3oc3cccc(-c5nc(-c6ccccc6)nc(-c6ccc7c(ccc8ccccc87)c6)n5)c34)cc2)cc1 ; c1ccc(-c2ccc(-c3ccc4oc5cccc(-c6nc(-c7ccccc7)nc(-c7ccc8c(ccc9ccccc98)c7)n6)c5c4c3)cc2)cc1]

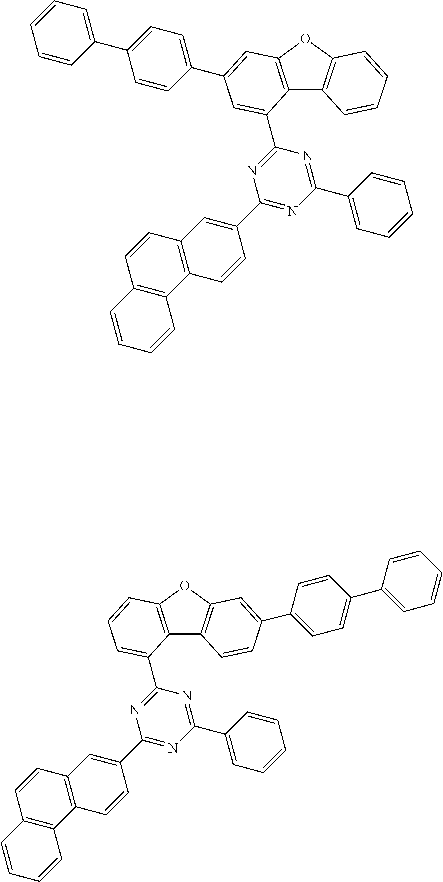 [SMILES: c1ccc(-c2ccc(-c3cc(-c4nc(-c5ccccc5)nc(-c5ccc6c(ccc7ccccc76)c5)n4)c4c(c3)oc3ccccc34)cc2)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1]

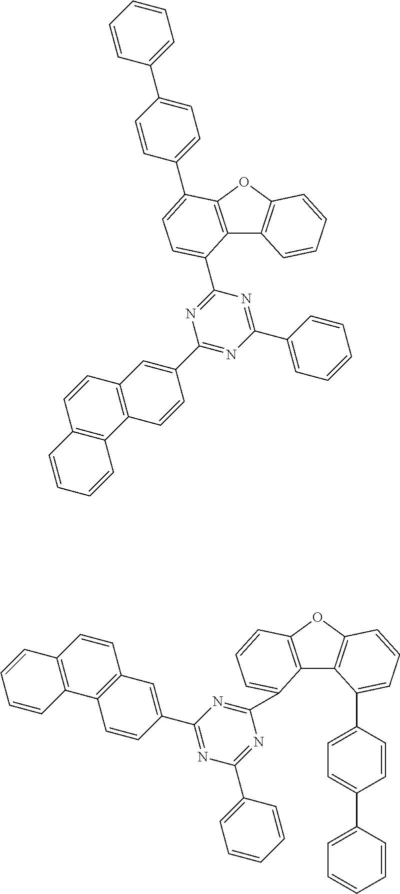 [SMILES: c1ccc(-c2ccc(-c3ccc(-c4nc(-c5ccccc5)nc(-c5ccc6c(ccc7ccccc76)c5)n4)c4c3oc3ccccc34)cc2)cc1 ; c1ccc(-c2ccc(-c3cccc4oc5cccc(-c6nc(-c7ccccc7)nc(-c7ccc8c(ccc9ccccc98)c7)n6)c5c34)cc2)cc1]

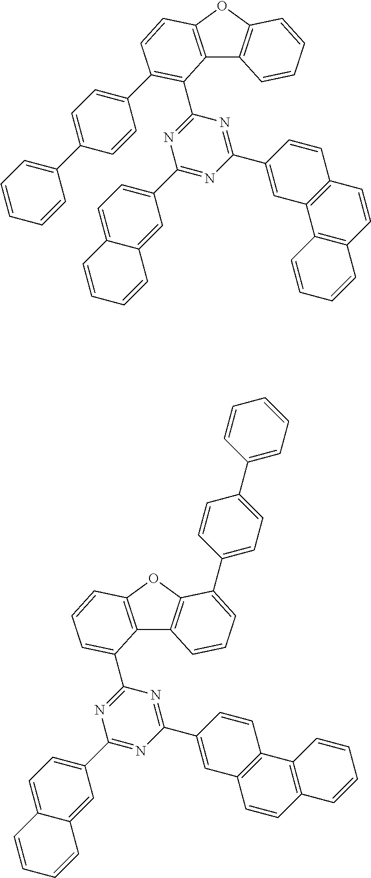 [SMILES: c1ccc(-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3cccc4c3oc3cccc(-c5nc(-c6ccc7ccccc7c6)nc(-c6ccc7c(ccc8ccccc87)c6)n5)c34)cc2)cc1]

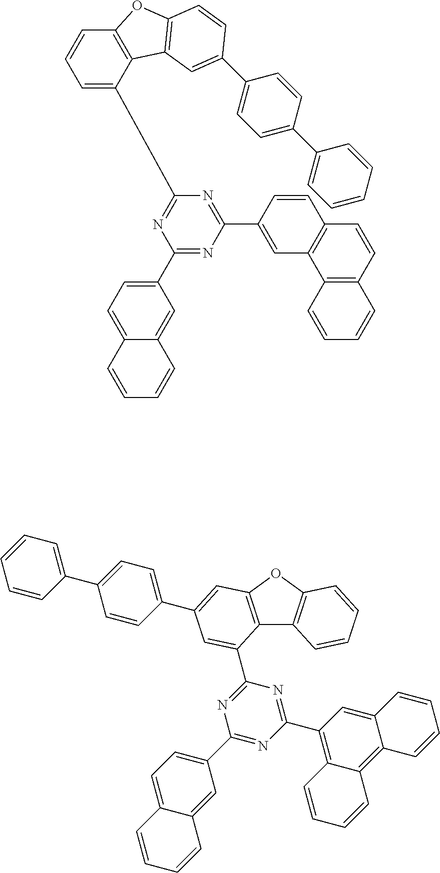 [SMILES: c1ccc(-c2ccc(-c3ccc4oc5cccc(-c6nc(-c7ccc8ccccc8c7)nc(-c7ccc8ccc9ccccc9c8c7)n6)c5c4c3)cc2)cc1 ; c1ccc(-c2ccc(-c3cc(-c4nc(-c5ccc6ccccc6c5)nc(-c5cc6ccccc6c6ccccc56)n4)c4c(c3)oc3ccccc34)cc2)cc1]

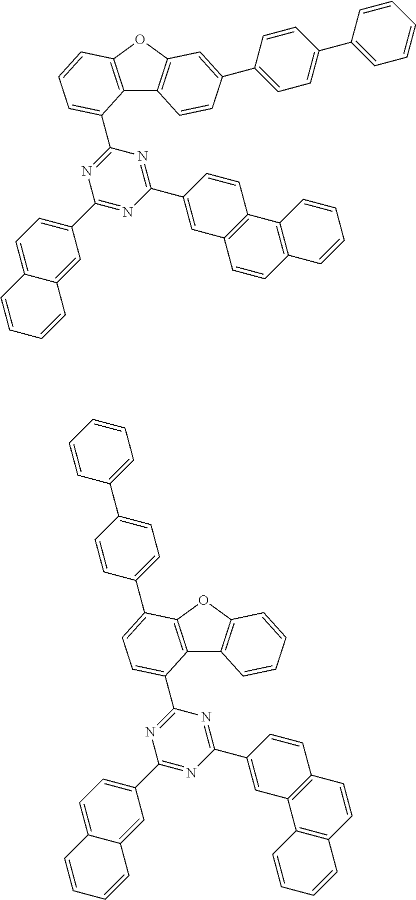 [SMILES: c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1 ; c1ccc(-c2ccc(-c3ccc(-c4nc(-c5ccc6ccccc6c5)nc(-c5ccc6ccc7ccccc7c6c5)n4)c4c3oc3ccccc34)cc2)cc1]

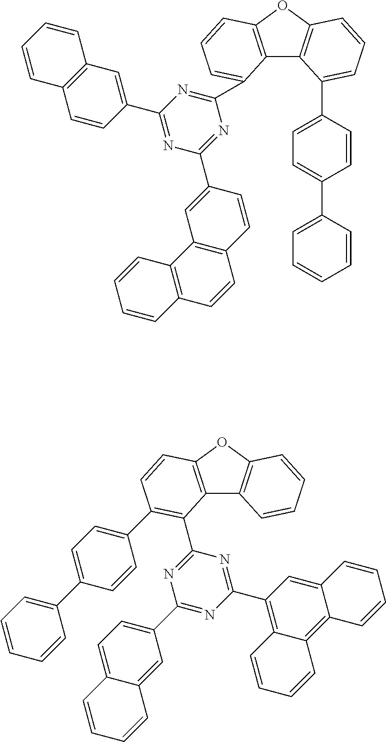 [SMILES: c1ccc(-c2ccc(-c3cccc4oc5cccc(-c6nc(-c7ccc8ccccc8c7)nc(-c7ccc8ccc9ccccc9c8c7)n6)c5c34)cc2)cc1 ; c1ccc(-c2ccc(-c3ccc4oc5ccccc5c4c3-c3nc(-c4ccc5ccccc5c4)nc(-c4cc5ccccc5c5ccccc45)n3)cc2)cc1]

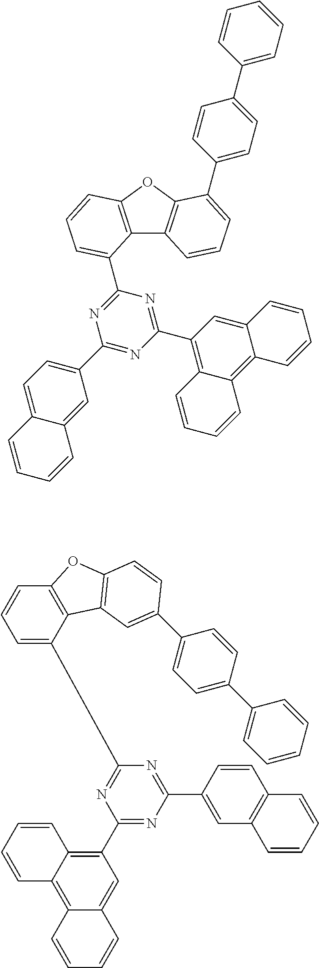 [SMILES: c1ccc(-c2ccc(-c3cccc4c3oc3cccc(-c5nc(-c6ccc7ccccc7c6)nc(-c6cc7ccccc7c7ccccc67)n5)c34)cc2)cc1 ; c1ccc(-c2ccc(-c3ccc4oc5cccc(-c6nc(-c7ccc8ccccc8c7)nc(-c7cc8ccccc8c8ccccc78)n6)c5c4c3)cc2)cc1]

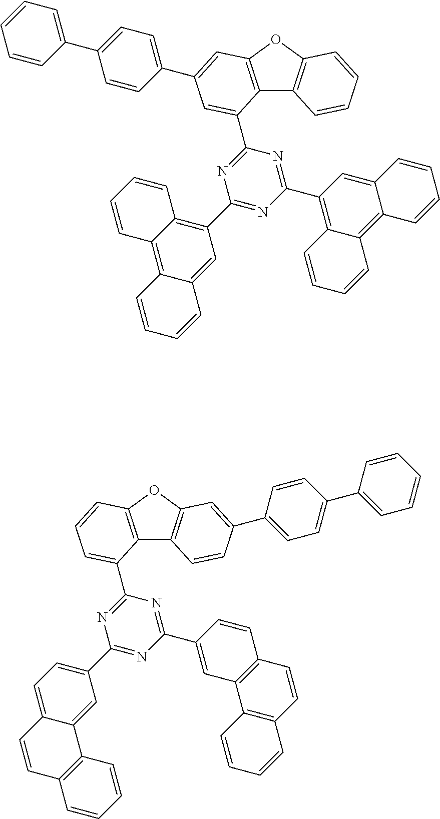 [SMILES: c1ccc(-c2ccc(-c3cc(-c4nc(-c5cc6ccccc6c6ccccc56)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccc7ccc8ccccc8c7c6)nc(-c6ccc7ccc8ccccc8c7c6)n5)c34)cc2)cc1]

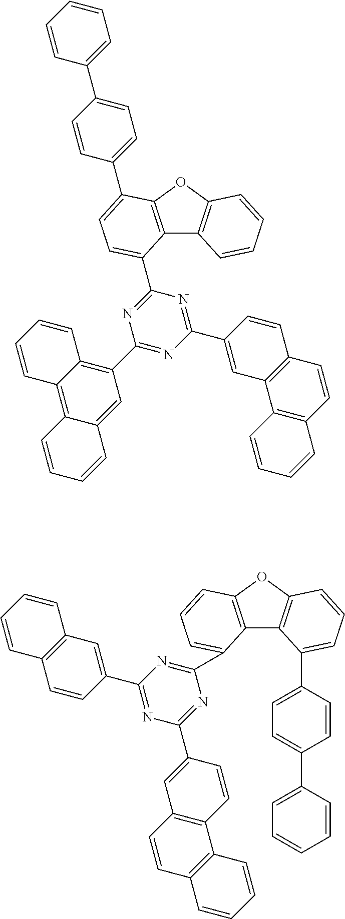 [SMILES: c1ccc(-c2ccc(-c3ccc(-c4nc(-c5ccc6ccc7ccccc7c6c5)nc(-c5cc6ccccc6c6ccccc56)n4)c4c3oc3ccccc34)cc2)cc1 ; c1ccc(-c2ccc(-c3cccc4oc5cccc(-c6nc(-c7ccc8ccccc8c7)nc(-c7ccc8c(ccc9ccccc98)c7)n6)c5c34)cc2)cc1]

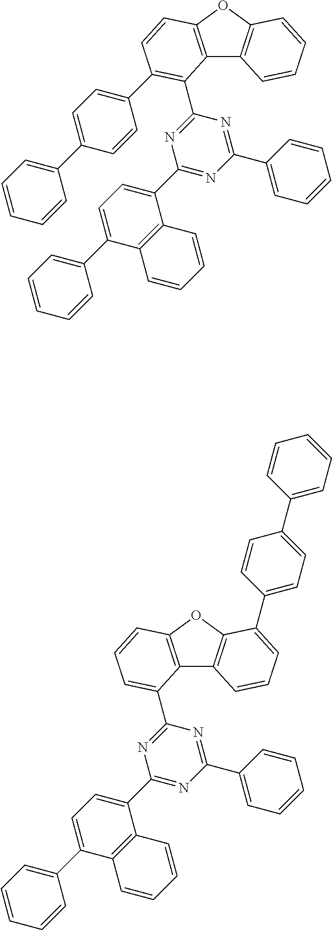 [SMILES: c1ccc(-c2ccc(-c3ccc4oc5ccccc5c4c3-c3nc(-c4ccccc4)nc(-c4ccc(-c5ccccc5)c5ccccc45)n3)cc2)cc1 ; c1ccc(-c2ccc(-c3cccc4c3oc3cccc(-c5nc(-c6ccccc6)nc(-c6ccc(-c7ccccc7)c7ccccc67)n5)c34)cc2)cc1]

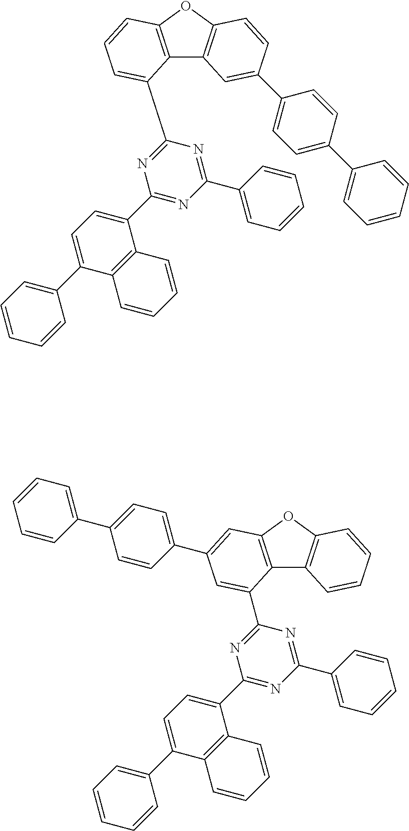 [SMILES: c1ccc(-c2ccc(-c3ccc4oc5cccc(-c6nc(-c7ccccc7)nc(-c7ccc(-c8ccccc8)c8ccccc78)n6)c5c4c3)cc2)cc1 ; c1ccc(-c2ccc(-c3cc(-c4nc(-c5ccccc5)nc(-c5ccc(-c6ccccc6)c6ccccc56)n4)c4c(c3)oc3ccccc34)cc2)cc1]

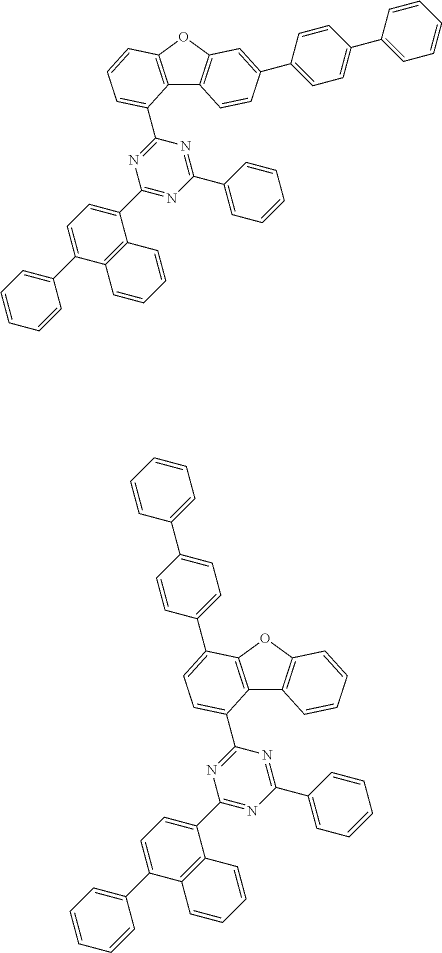 [SMILES: c1ccc(-c2ccc(-c3ccc4c(c3)oc3cccc(-c5nc(-c6ccccc6)nc(-c6ccc(-c7ccccc7)c7ccccc67)n5)c34)cc2)cc1 ; c1ccc(-c2ccc(-c3ccc(-c4nc(-c5ccccc5)nc(-c5ccc(-c6ccccc6)c6ccccc56)n4)c4c3oc3ccccc34)cc2)cc1]

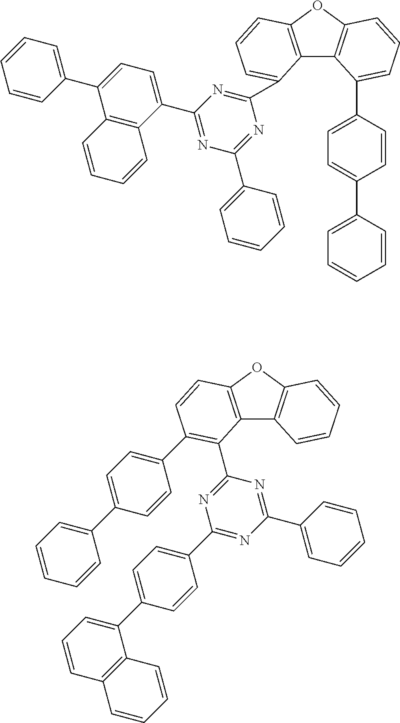 [SMILES: c1ccc(-c2ccc(-c3cccc4oc5cccc(-c6nc(-c7ccccc7)nc(-c7ccc(-c8ccccc8)c8ccccc78)n6)c5c34)cc2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1]

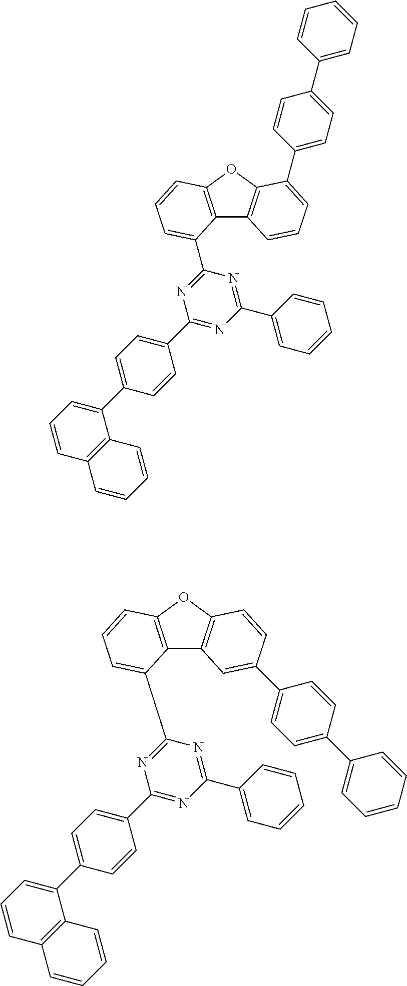 [SMILES: c1ccc(-c2ccc(-c3cccc4c3oc3cccc(-c5nc(-c6ccccc6)nc(-c6ccc(-c7cccc8ccccc78)cc6)n5)c34)cc2)cc1 ; c1ccc(-c2ccc(-c3ccc4oc5cccc(-c6nc(-c7ccccc7)nc(-c7ccc(-c8cccc9ccccc89)cc7)n6)c5c4c3)cc2)cc1]

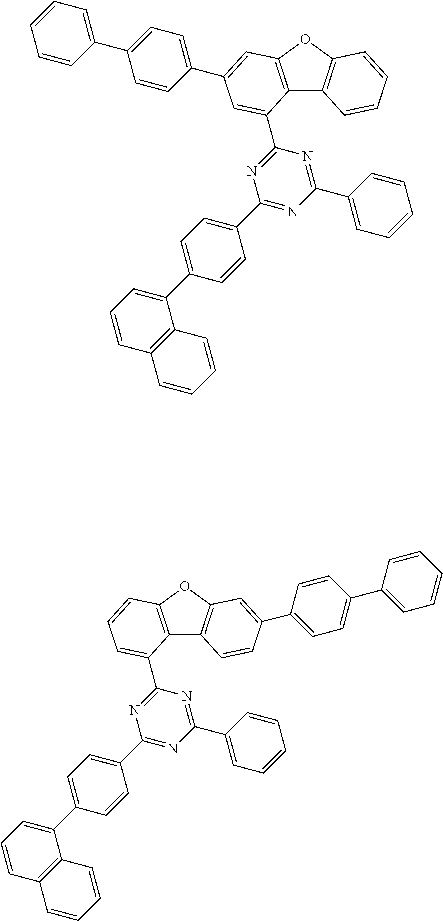 [SMILES: c1ccc(-c2ccc(-c3cc(-c4nc(-c5ccccc5)nc(-c5ccc(-c6cccc7ccccc67)cc5)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7cccc8ccccc78)cc6)n5)c34)cc2)cc1]

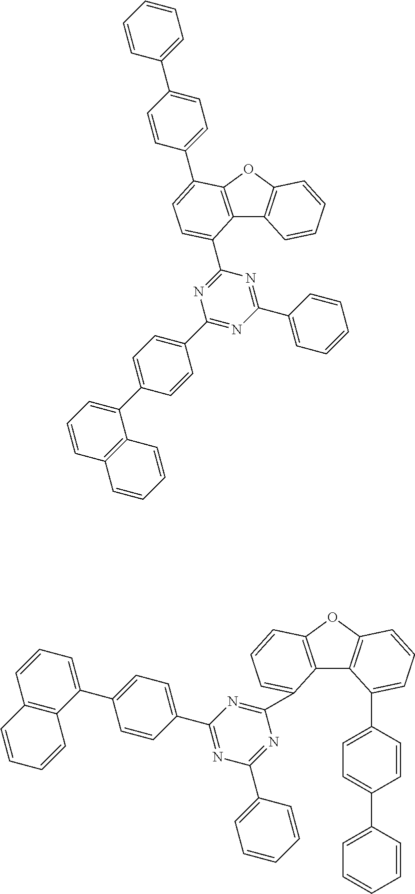 [SMILES: c1ccc(-c2ccc(-c3ccc(-c4nc(-c5ccccc5)nc(-c5ccc(-c6cccc7ccccc67)cc5)n4)c4c3oc3ccccc34)cc2)cc1 ; c1ccc(-c2ccc(-c3cccc4oc5cccc(-c6nc(-c7ccccc7)nc(-c7ccc(-c8cccc9ccccc89)cc7)n6)c5c34)cc2)cc1]

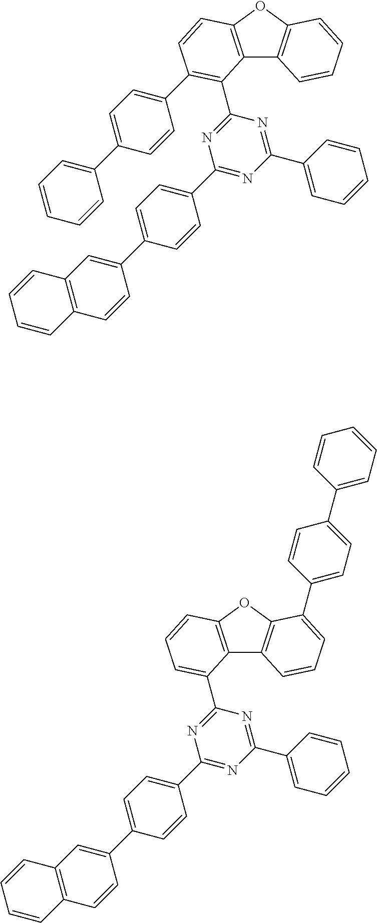 [SMILES: c1ccc(-c2ccc(-c3ccc4oc5ccccc5c4c3-c3nc(-c4ccccc4)nc(-c4ccc(-c5ccc6ccccc6c5)cc4)n3)cc2)cc1 ; c1ccc(-c2ccc(-c3cccc4c3oc3cccc(-c5nc(-c6ccccc6)nc(-c6ccc(-c7ccc8ccccc8c7)cc6)n5)c34)cc2)cc1]

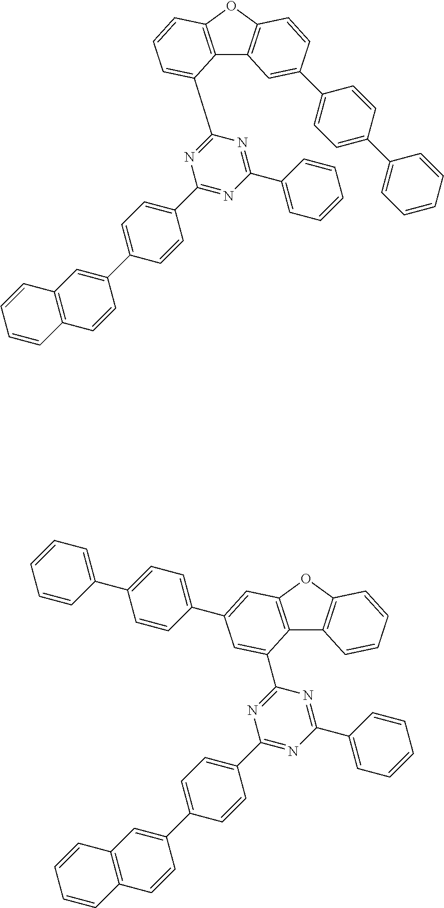 [SMILES: c1ccc(-c2ccc(-c3ccc4oc5cccc(-c6nc(-c7ccccc7)nc(-c7ccc(-c8ccc9ccccc9c8)cc7)n6)c5c4c3)cc2)cc1 ; c1ccc(-c2ccc(-c3cc(-c4nc(-c5ccccc5)nc(-c5ccc(-c6ccc7ccccc7c6)cc5)n4)c4c(c3)oc3ccccc34)cc2)cc1]

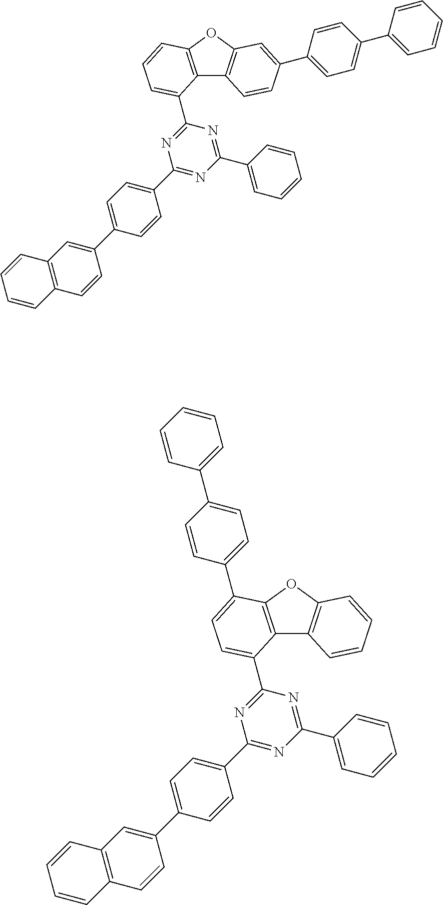 [SMILES: c1ccc(-c2ccc(-c3ccc4c(c3)oc3cccc(-c5nc(-c6ccccc6)nc(-c6ccc(-c7ccc8ccccc8c7)cc6)n5)c34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1]

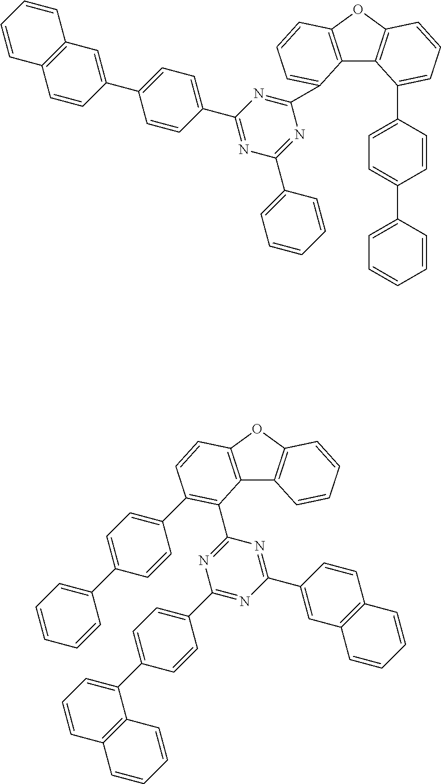 [SMILES: c1ccc(-c2ccc(-c3cccc4oc5cccc(-c6nc(-c7ccccc7)nc(-c7ccc(-c8ccc9ccccc9c8)cc7)n6)c5c34)cc2)cc1 ; c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

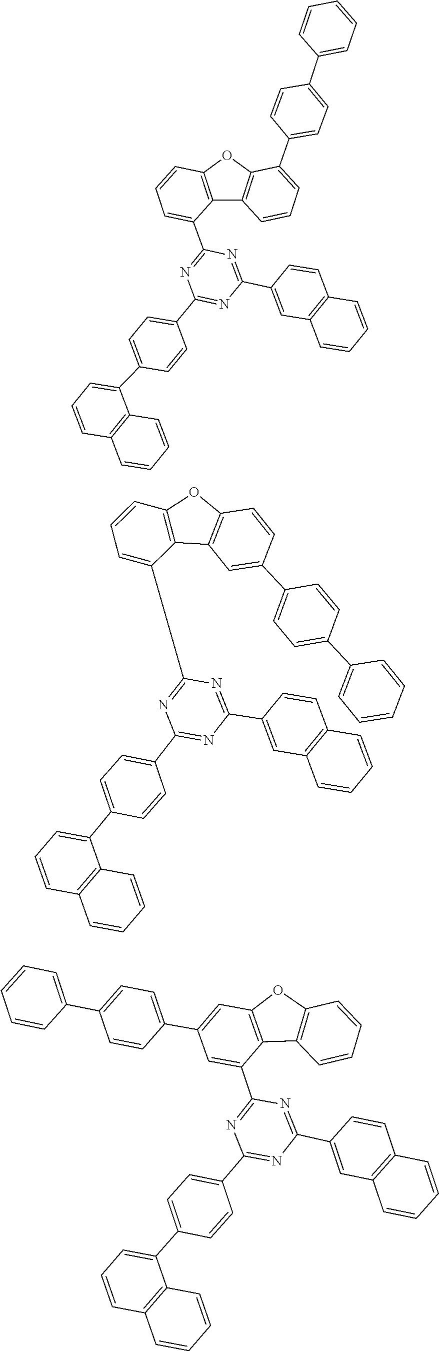

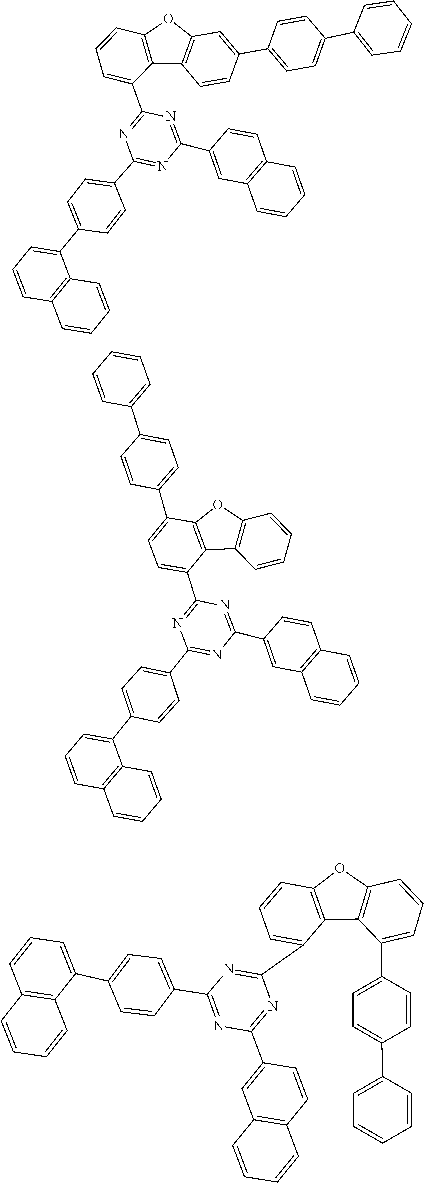

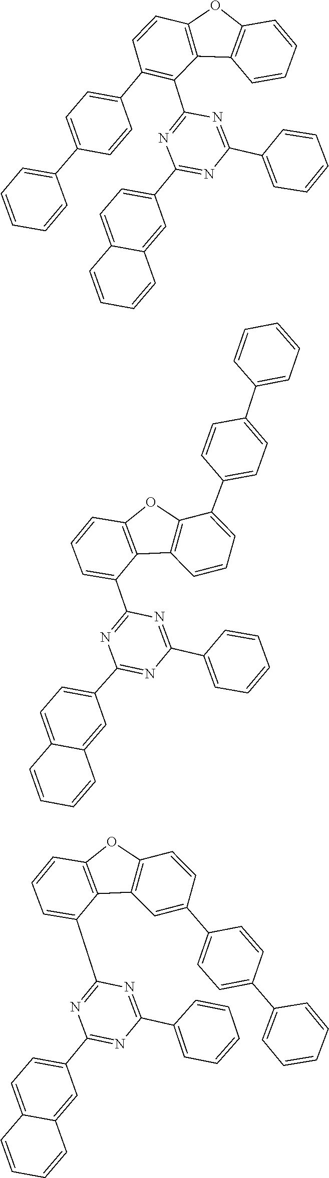 [SMILES: c1ccc(-c2ccc(-c3ccc4oc5ccccc5c4c3-c3nc(-c4ccccc4)nc(-c4ccc5ccccc5c4)n3)cc2)cc1 ; c1ccc(-c2ccc(-c3cccc4c3oc3cccc(-c5nc(-c6ccccc6)nc(-c6ccc7ccccc7c6)n5)c34)cc2)cc1 ; c1ccc(-c2ccc(-c3ccc4oc5cccc(-c6nc(-c7ccccc7)nc(-c7ccc8ccccc8c7)n6)c5c4c3)cc2)cc1]

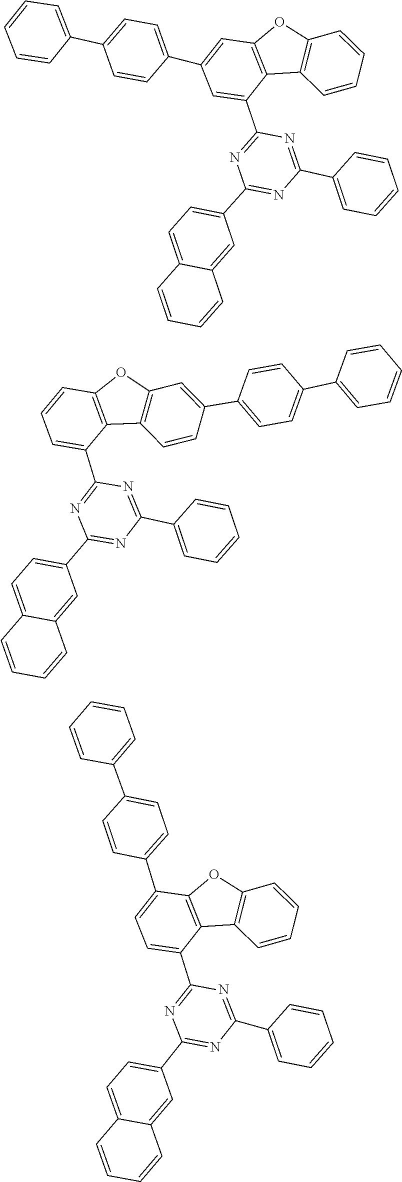 [SMILES: c1ccc(-c2ccc(-c3cc(-c4nc(-c5ccccc5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1]

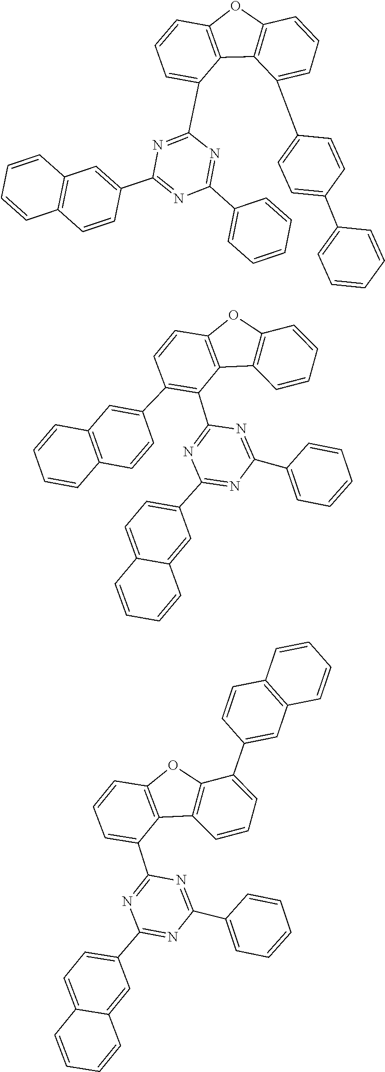 [SMILES: c1ccc(-c2ccc(-c3cccc4oc5cccc(-c6nc(-c7ccccc7)nc(-c7ccc8ccccc8c7)n6)c5c34)cc2)cc1 ; c1ccc(-c2nc(-c3ccc4ccccc4c3)nc(-c3c(-c4ccc5ccccc5c4)ccc4oc5ccccc5c34)n2)cc1 ; c1ccc(-c2nc(-c3ccc4ccccc4c3)nc(-c3cccc4oc5c(-c6ccc7ccccc7c6)cccc5c34)n2)cc1]

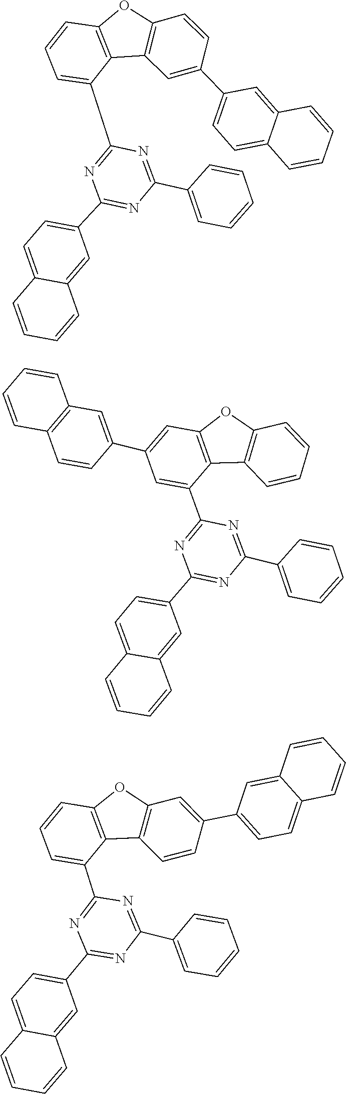

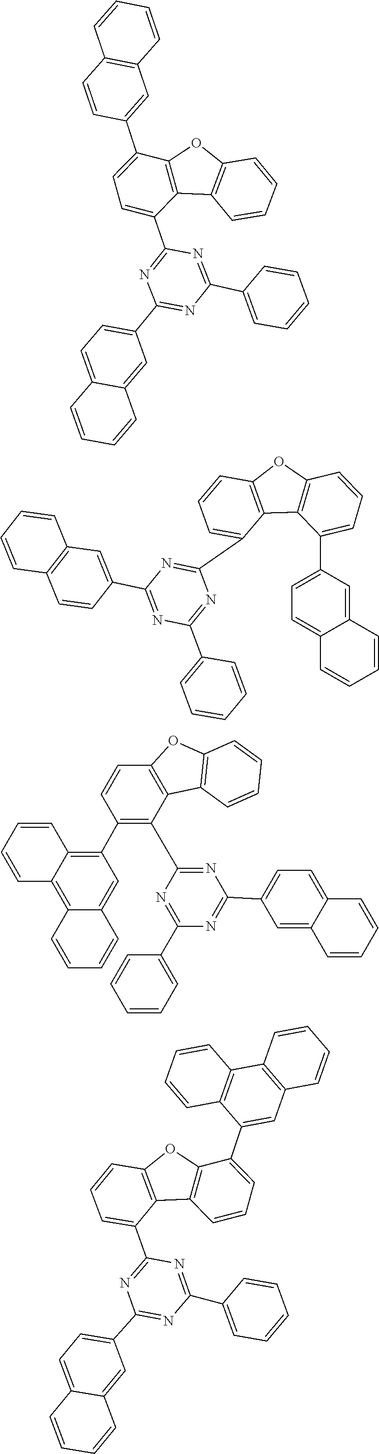

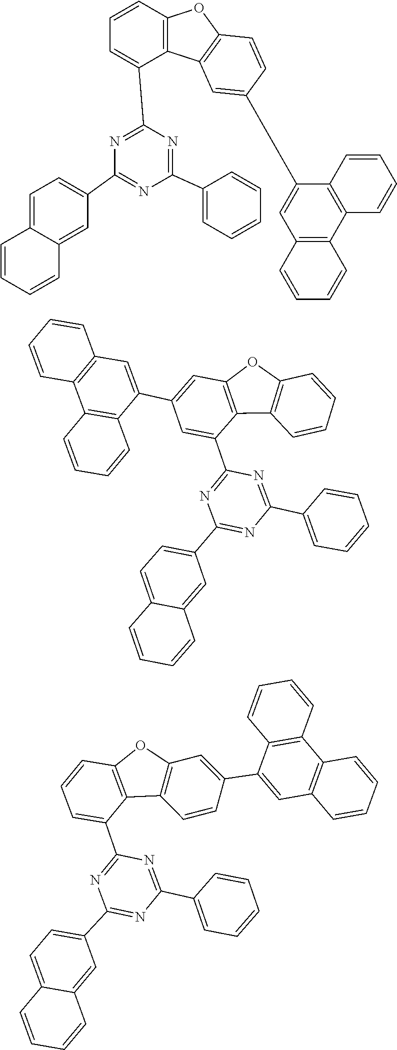

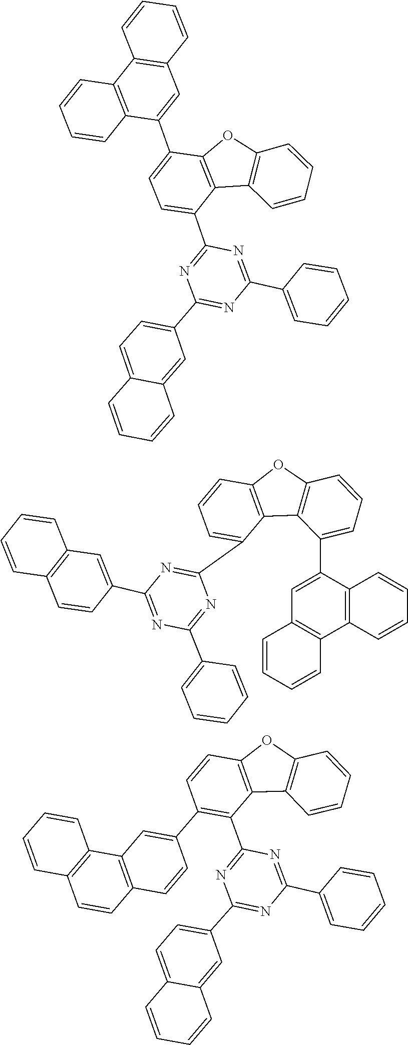

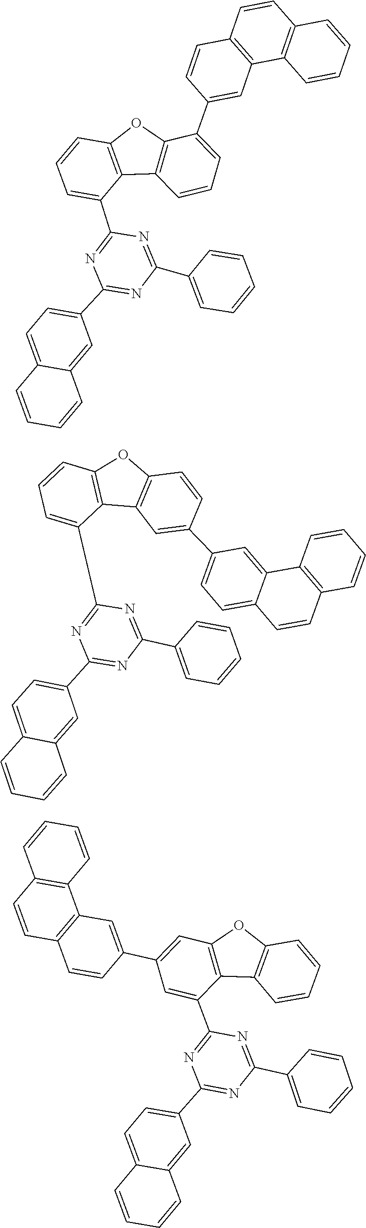

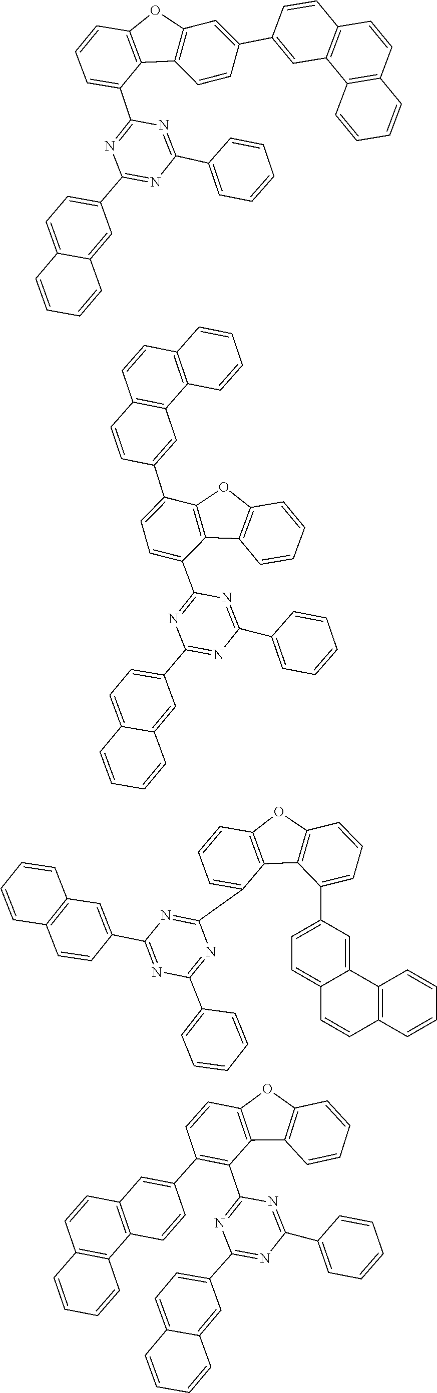

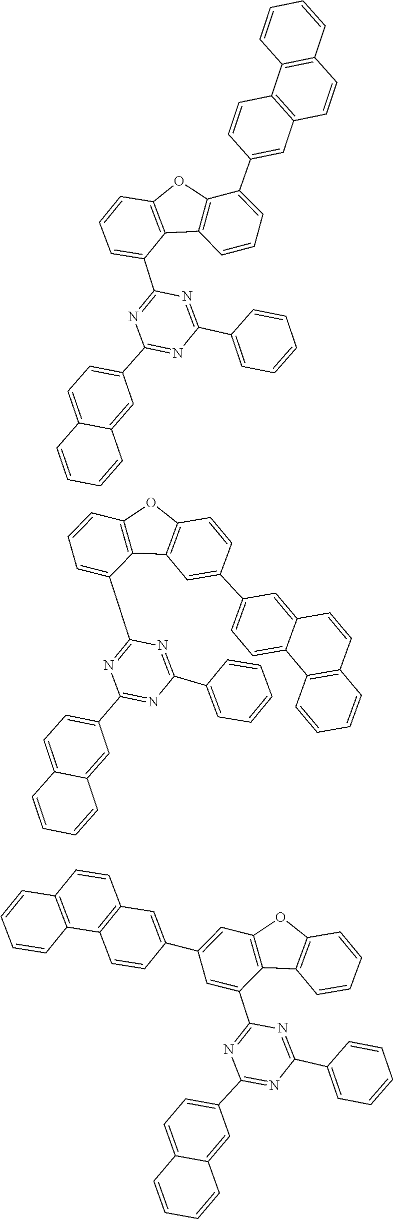

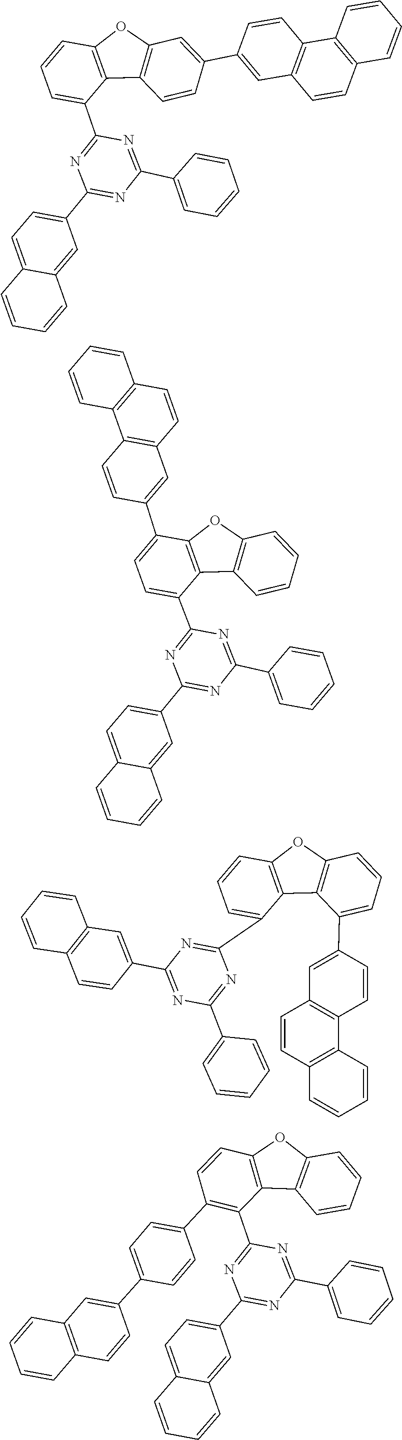

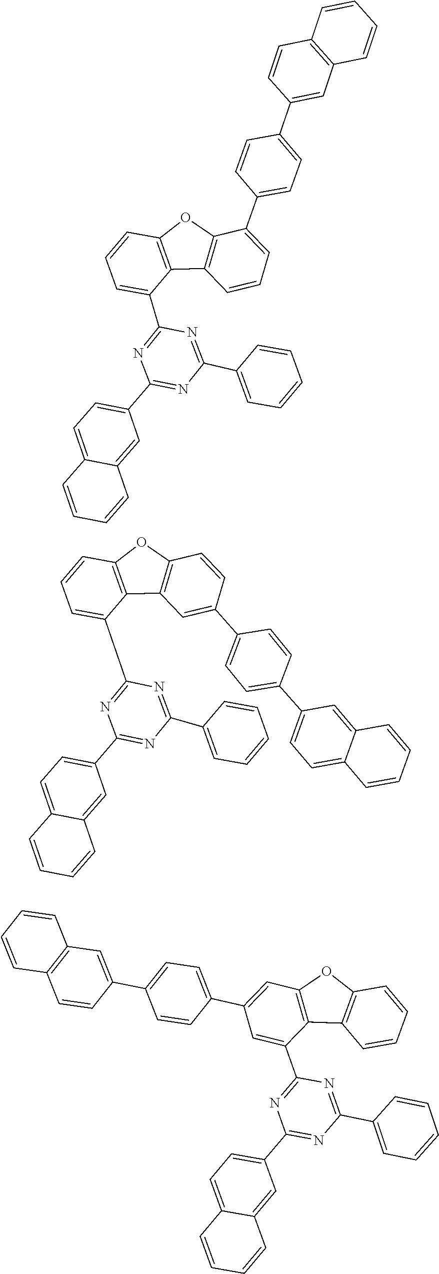

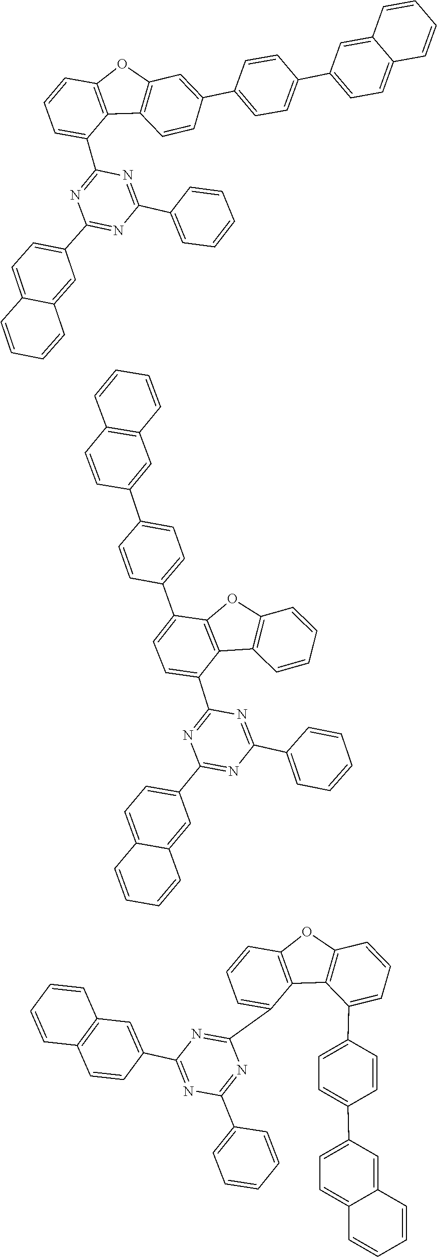 [SMILES: c1ccc(-c2nc(-c3ccc4ccccc4c3)nc(-c3cccc4oc5cc(-c6ccc(-c7ccc8ccccc8c7)cc6)ccc5c34)n2)cc1 ; c1ccc(-c2nc(-c3ccc4ccccc4c3)nc(-c3ccc(-c4ccc(-c5ccc6ccccc6c5)cc4)c4oc5ccccc5c34)n2)cc1 ; c1ccc(-c2nc(-c3ccc4ccccc4c3)nc(-c3cccc4oc5cccc(-c6ccc(-c7ccc8ccccc8c7)cc6)c5c34)n2)cc1]

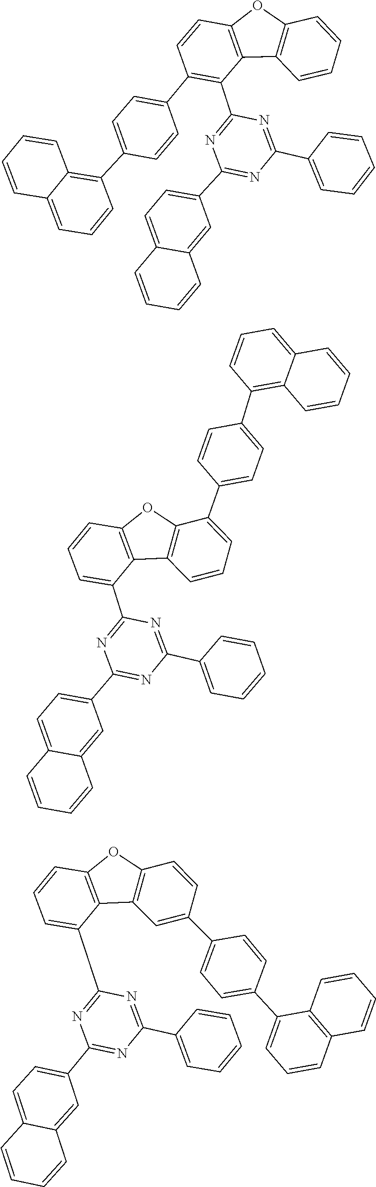 [SMILES: c1ccc(-c2nc(-c3ccc4ccccc4c3)nc(-c3c(-c4ccc(-c5cccc6ccccc56)cc4)ccc4oc5ccccc5c34)n2)cc1 ; c1ccc(-c2nc(-c3ccc4ccccc4c3)nc(-c3cccc4oc5c(-c6ccc(-c7cccc8ccccc78)cc6)cccc5c34)n2)cc1 ; c1ccc(-c2nc(-c3ccc4ccccc4c3)nc(-c3cccc4oc5ccc(-c6ccc(-c7cccc8ccccc78)cc6)cc5c34)n2)cc1]

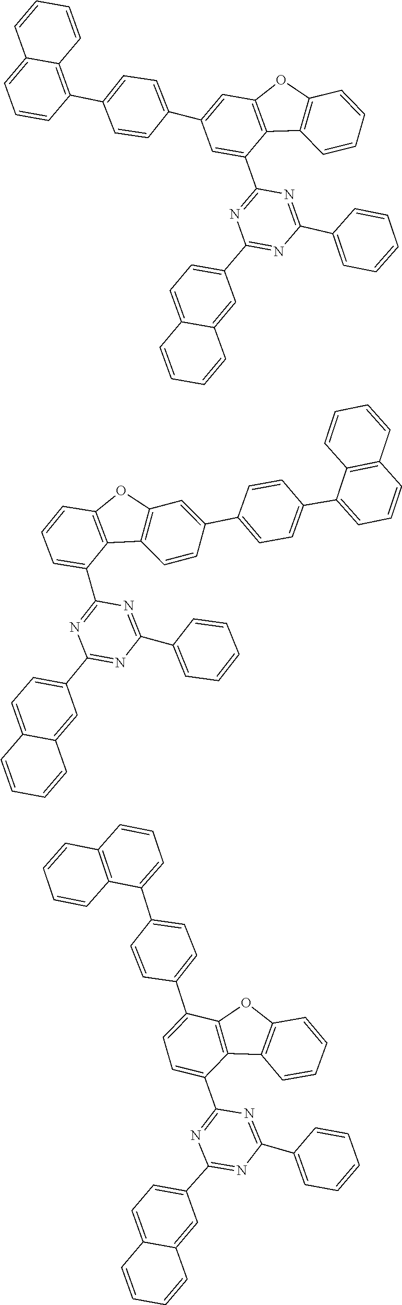 [SMILES: c1ccc(-c2nc(-c3ccc4ccccc4c3)nc(-c3cc(-c4ccc(-c5cccc6ccccc56)cc4)cc4oc5ccccc5c34)n2)cc1 ; c1ccc(-c2nc(-c3ccc4ccccc4c3)nc(-c3cccc4oc5cc(-c6ccc(-c7cccc8ccccc78)cc6)ccc5c34)n2)cc1 ; c1ccc(-c2nc(-c3ccc4ccccc4c3)nc(-c3ccc(-c4ccc(-c5cccc6ccccc56)cc4)c4oc5ccccc5c34)n2)cc1]

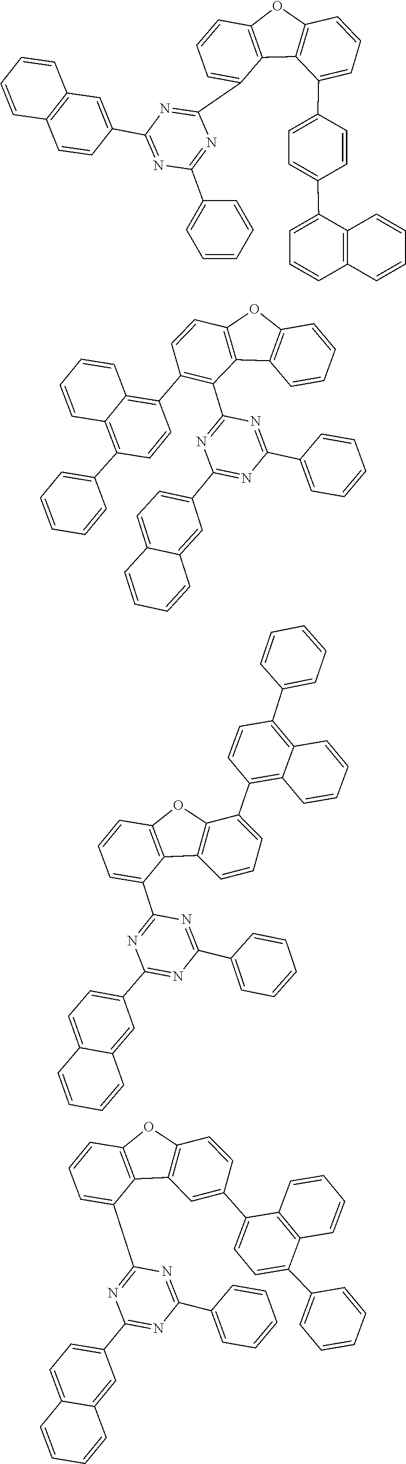

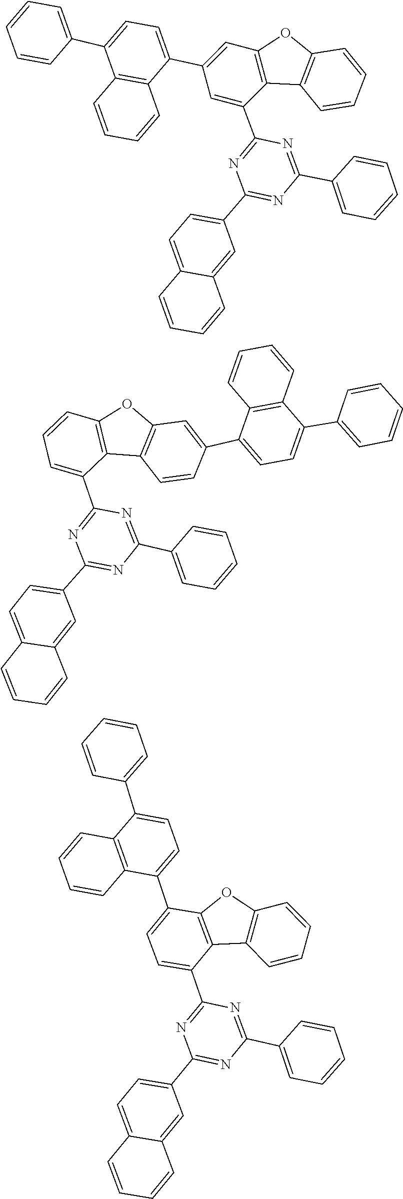

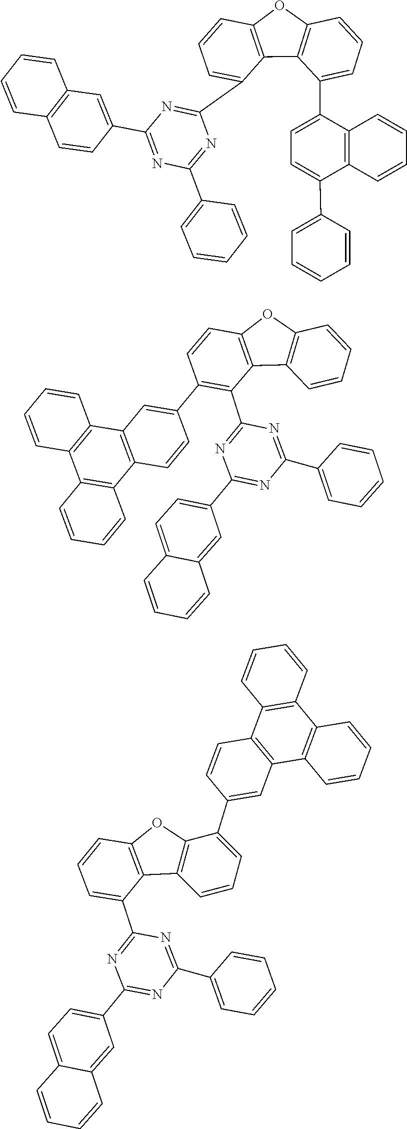

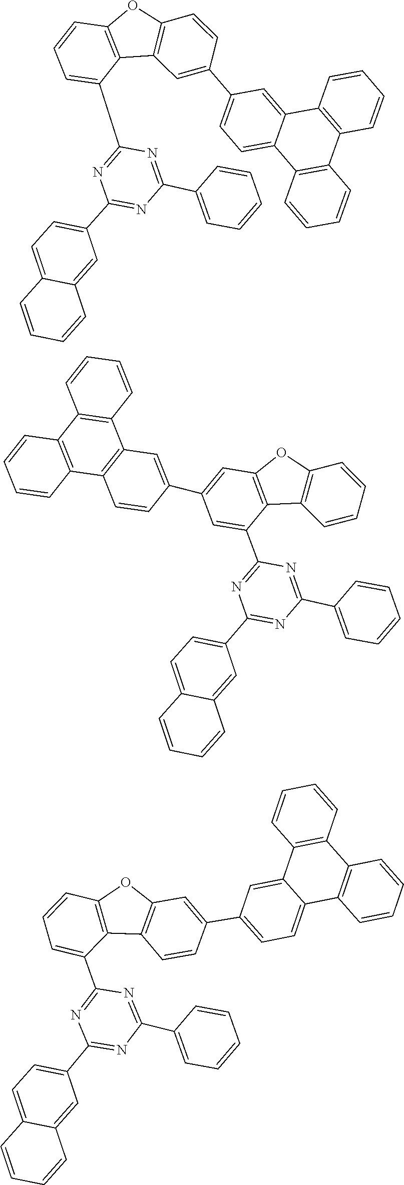

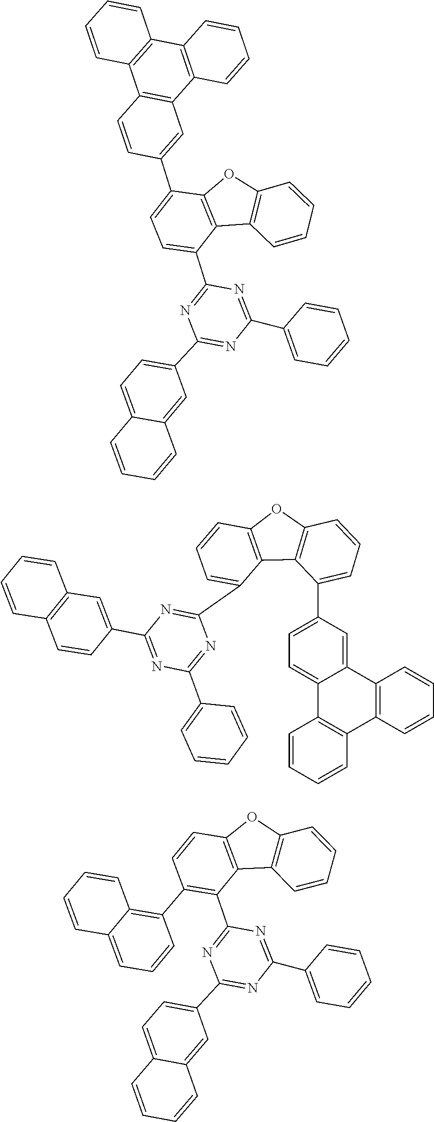

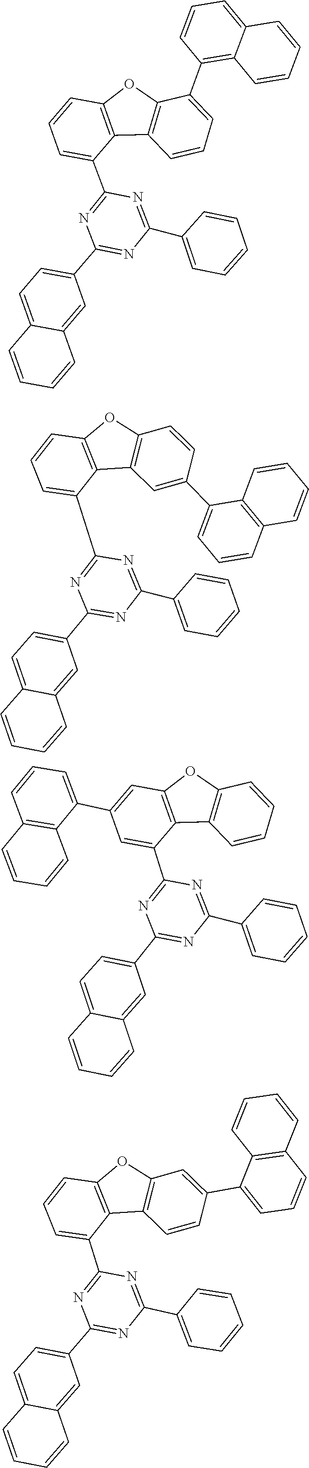

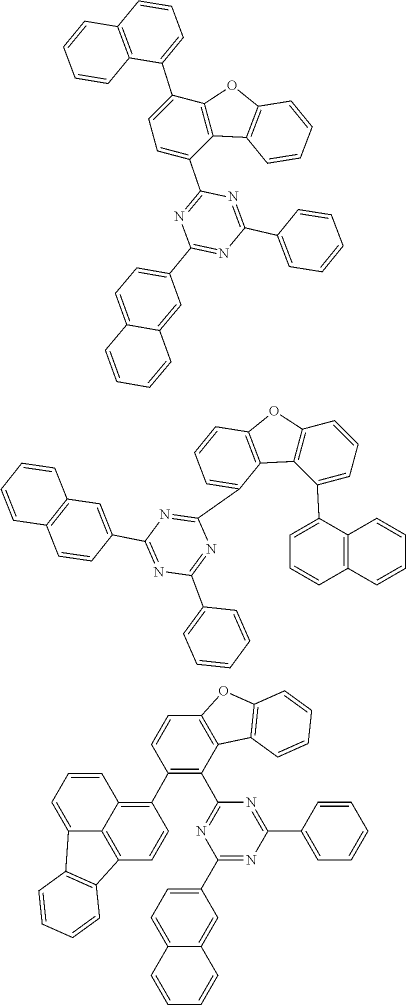

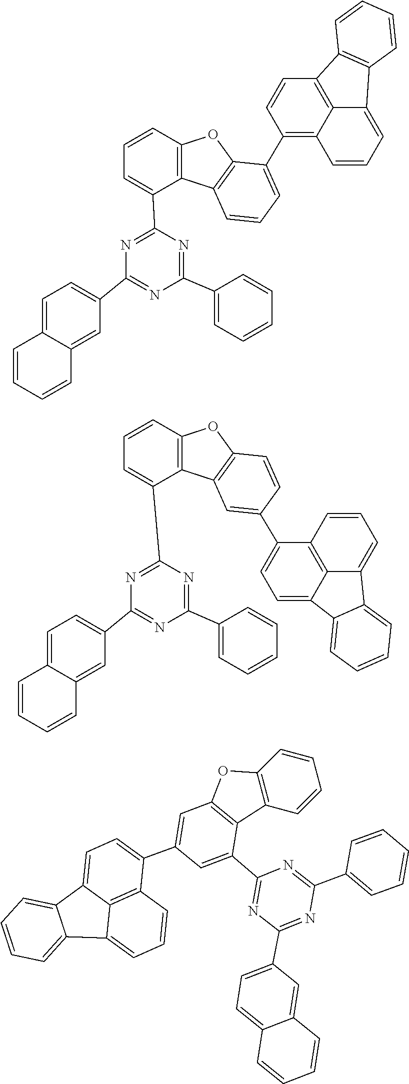 [SMILES: c1ccc(-c2nc(-c3ccc4ccccc4c3)nc(-c3cccc4oc5c(-c6ccc7c8c(cccc68)-c6ccccc6-7)cccc5c34)n2)cc1 ; c1ccc(-c2nc(-c3ccc4ccccc4c3)nc(-c3cccc4oc5ccc(-c6ccc7c8c(cccc68)-c6ccccc6-7)cc5c34)n2)cc1 ; c1ccc(-c2nc(-c3ccc4ccccc4c3)nc(-c3cc(-c4ccc5c6c(cccc46)-c4ccccc4-5)cc4oc5ccccc5c34)n2)cc1]

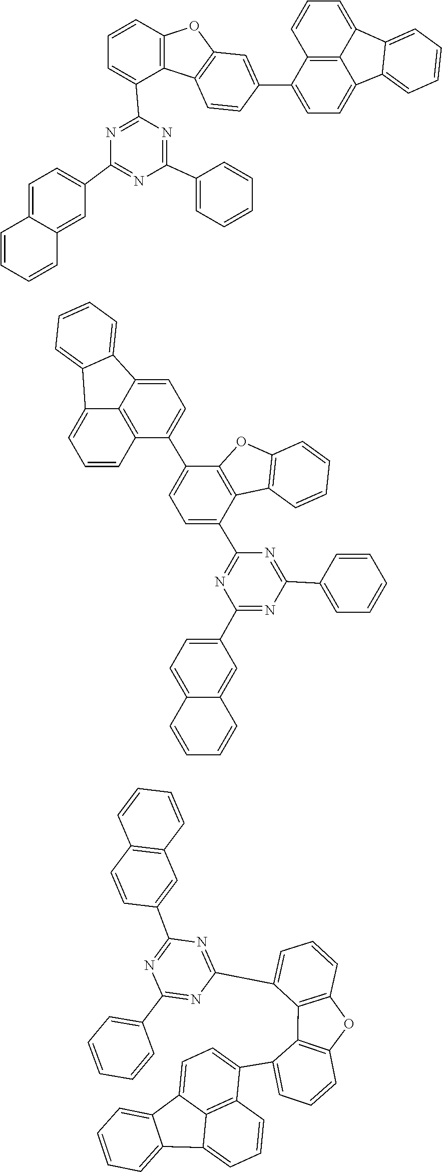

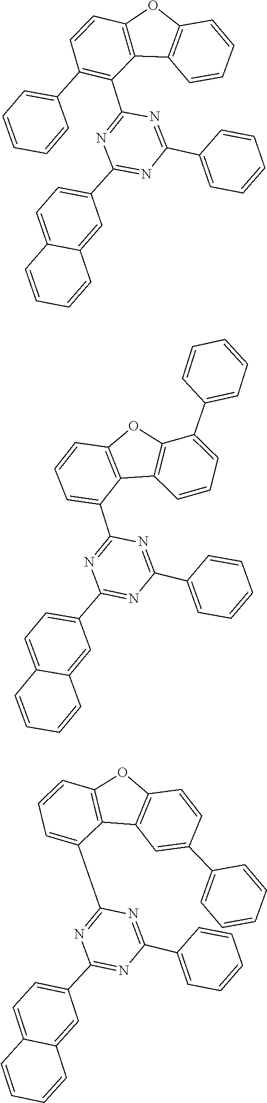 [SMILES: c1ccc(-c2nc(-c3ccc4ccccc4c3)nc(-c3c(-c4ccccc4)ccc4oc5ccccc5c34)n2)cc1 ; c1ccc(-c2nc(-c3ccc4ccccc4c3)nc(-c3cccc4oc5c(-c6ccccc6)cccc5c34)n2)cc1 ; c1ccc(-c2ccc3oc4cccc(-c5nc(-c6ccccc6)nc(-c6ccc7ccccc7c6)n5)c4c3c2)cc1]

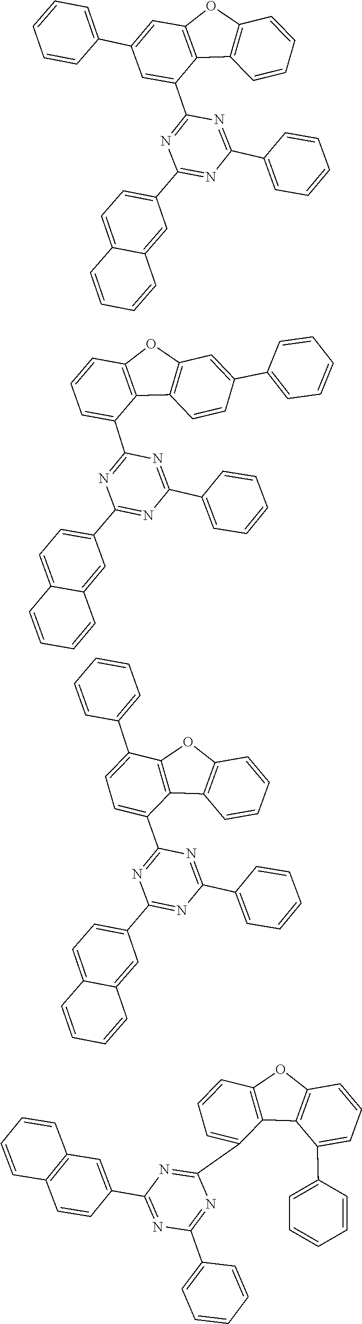

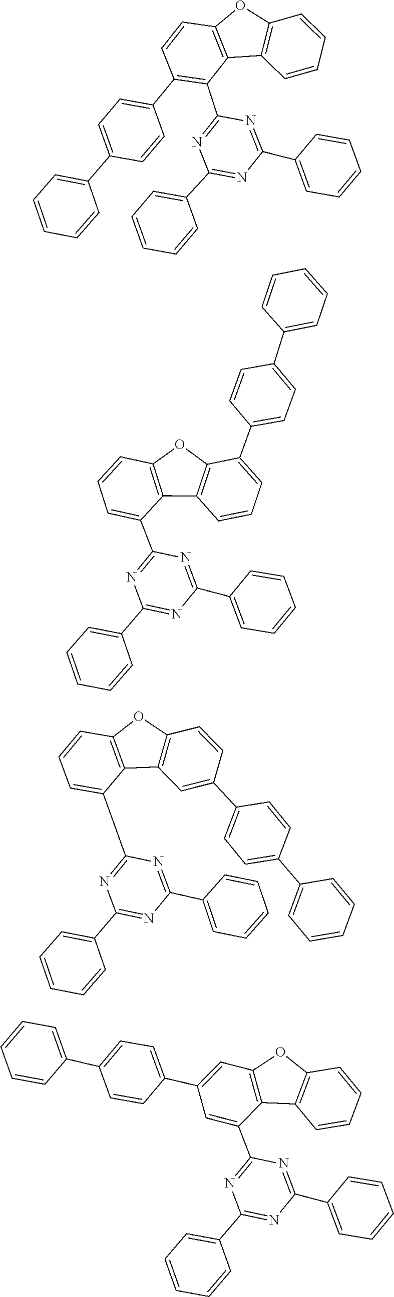

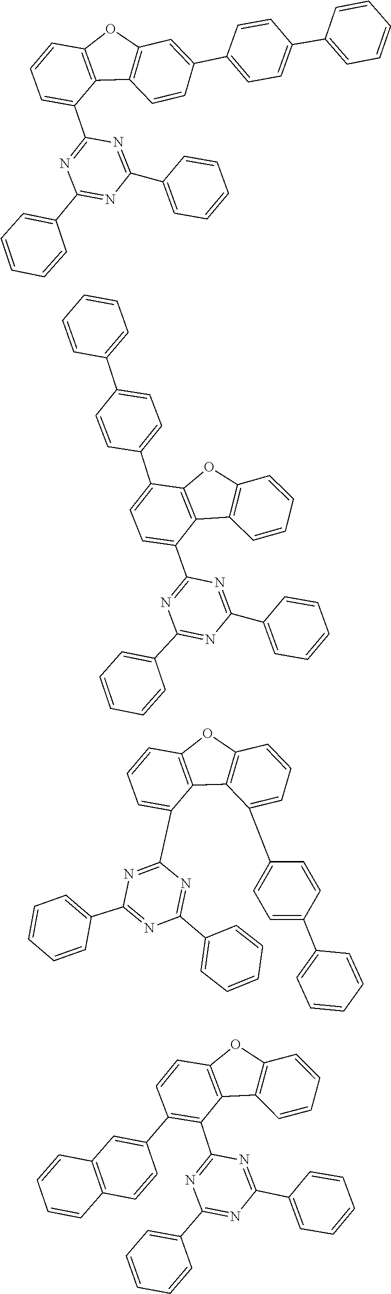

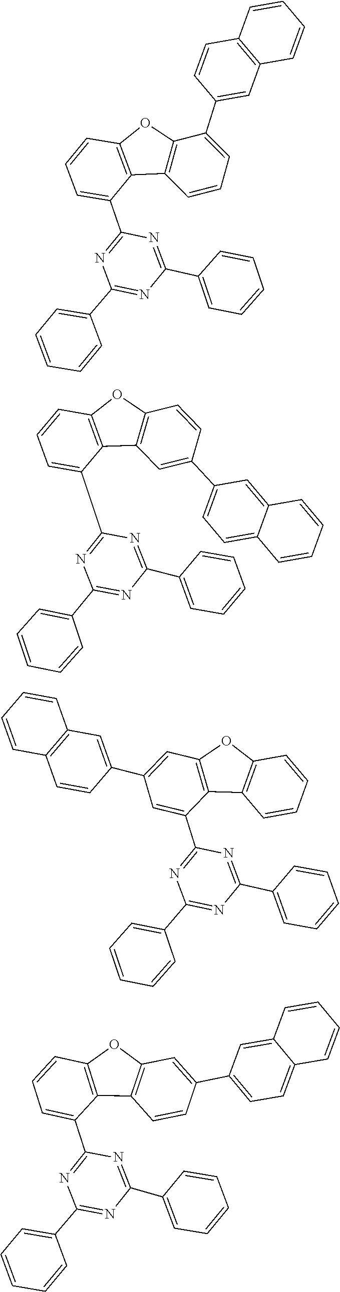

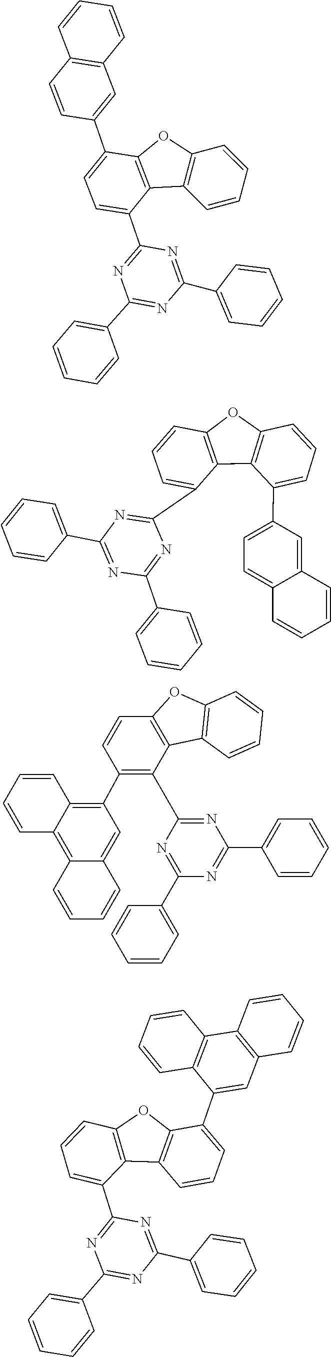

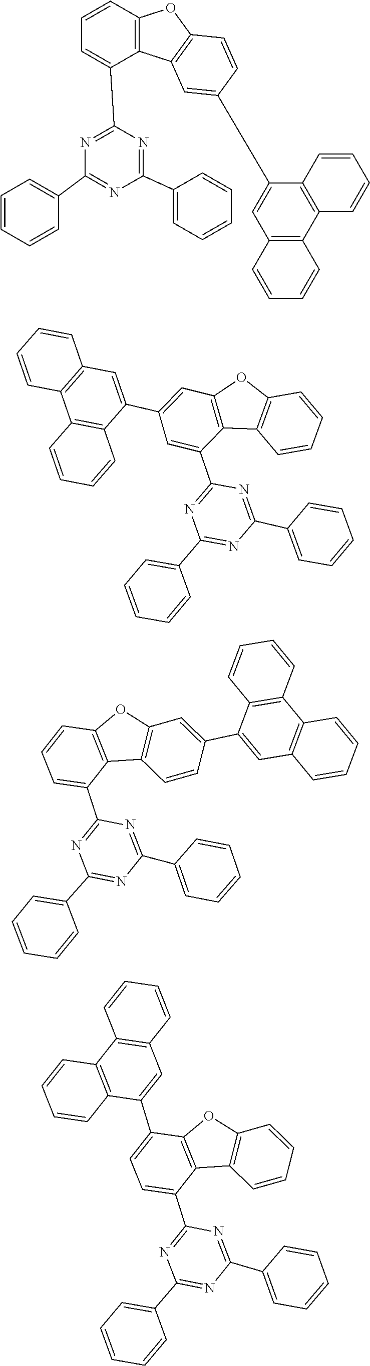 [SMILES: c1ccc(-c2nc(-c3ccccc3)nc(-c3cccc4oc5ccc(-c6cc7ccccc7c7ccccc67)cc5c34)n2)cc1 ; c1ccc(-c2nc(-c3ccccc3)nc(-c3cc(-c4cc5ccccc5c5ccccc45)cc4oc5ccccc5c34)n2)cc1 ; c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3ccc(-c4cc5ccccc5c5ccccc45)c4oc5ccccc5c34)n2)cc1]

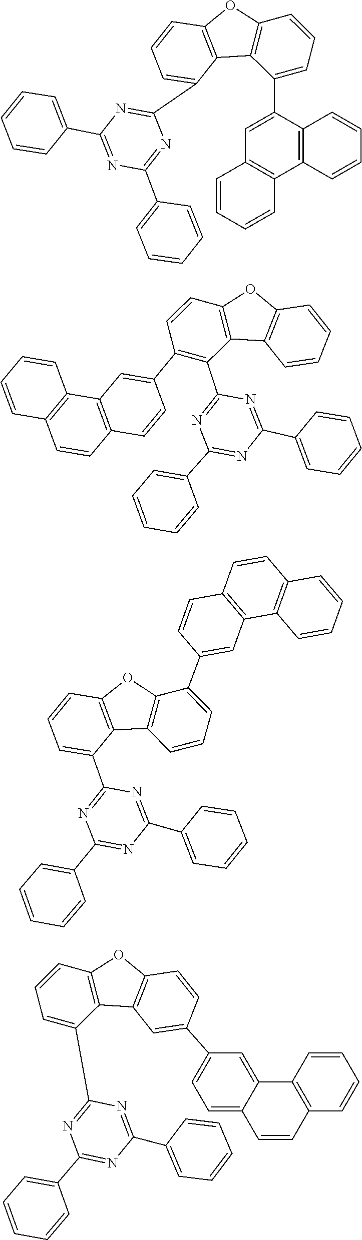

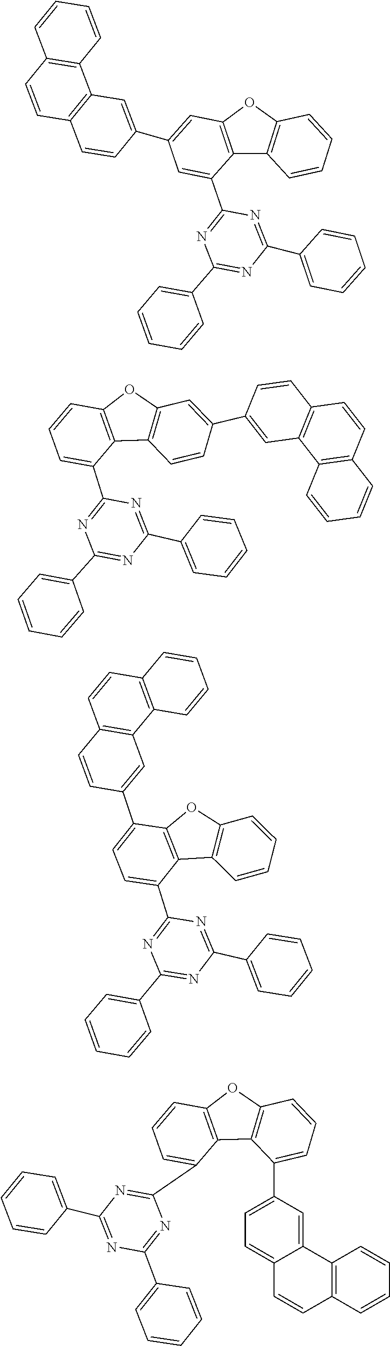 [SMILES: c1ccc(-c2nc(-c3ccccc3)nc(-c3cc(-c4ccc5ccc6ccccc6c5c4)cc4oc5ccccc5c34)n2)cc1 ; c1ccc(-c2nc(-c3ccccc3)nc(-c3cccc4oc5cc(-c6ccc7ccc8ccccc8c7c6)ccc5c34)n2)cc1 ; c1ccc(-c2nc(-c3ccccc3)nc(-c3ccc(-c4ccc5ccc6ccccc6c5c4)c4oc5ccccc5c34)n2)cc1 ; c1ccc(-c2nc(-c3ccccc3)nc(-c3cccc4oc5cccc(-c6ccc7ccc8ccccc8c7c6)c5c34)n2)cc1]

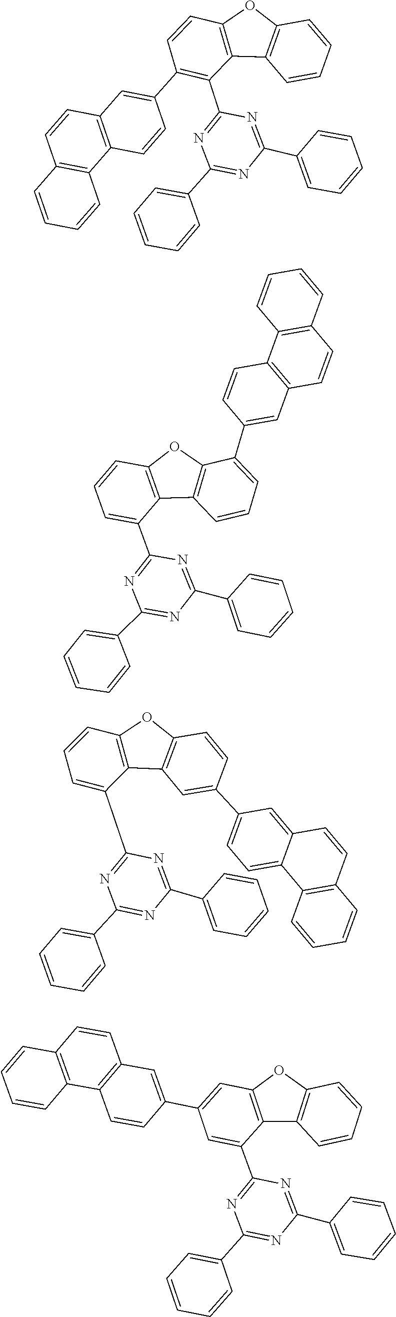

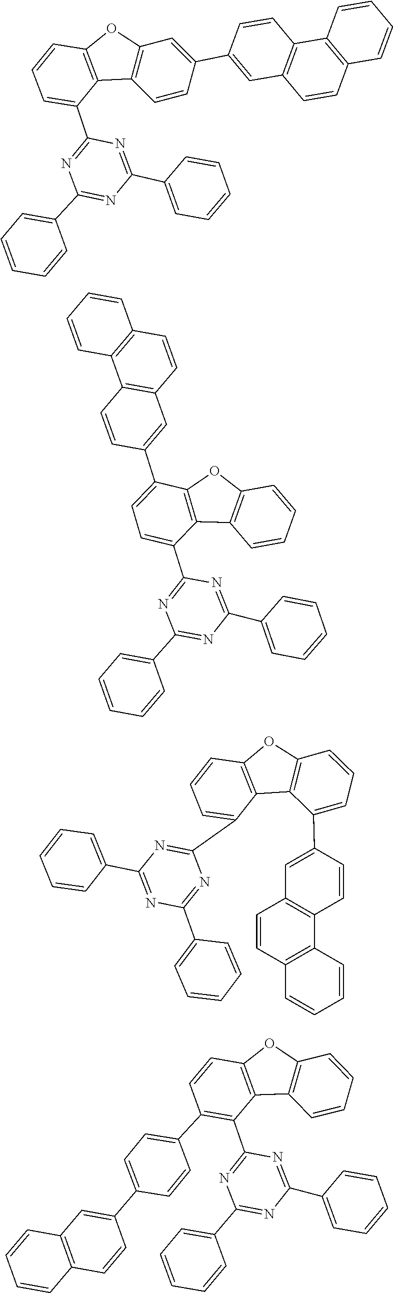

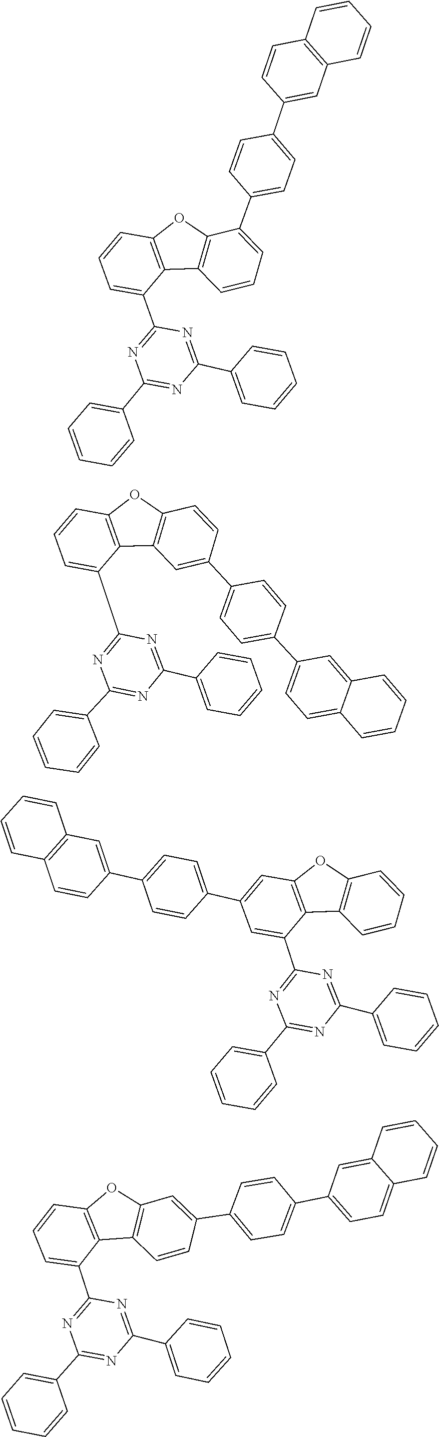

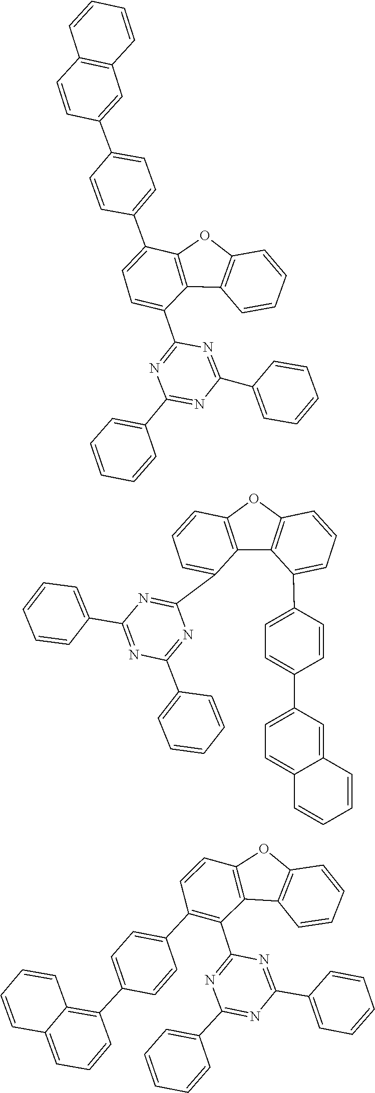

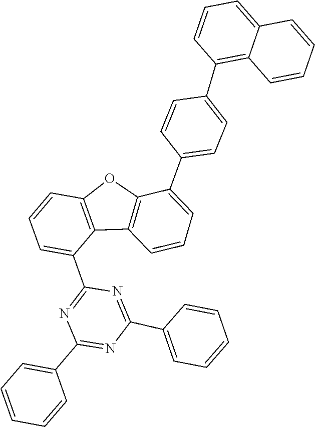 [SMILES: c1ccc(-c2nc(-c3ccccc3)nc(-c3cccc4oc5c(-c6ccc(-c7cccc8ccccc78)cc6)cccc5c34)n2)cc1]

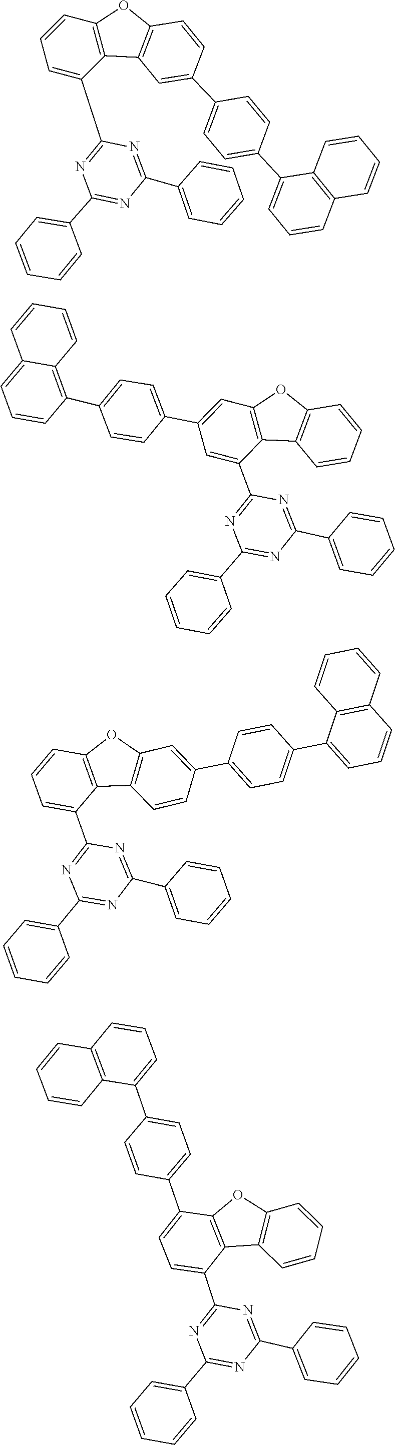

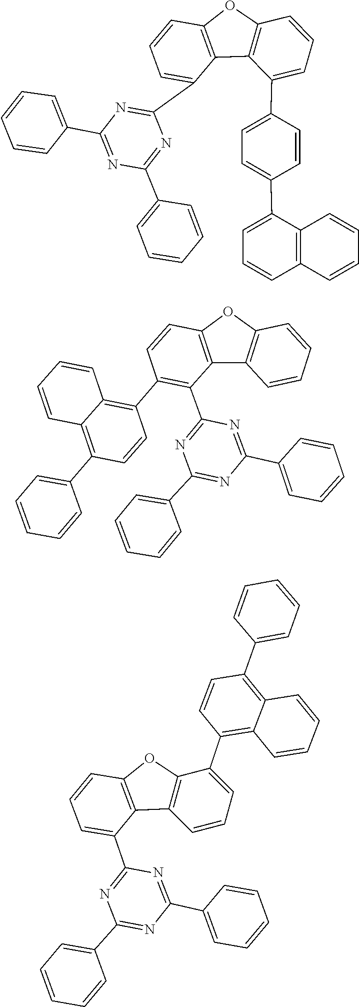

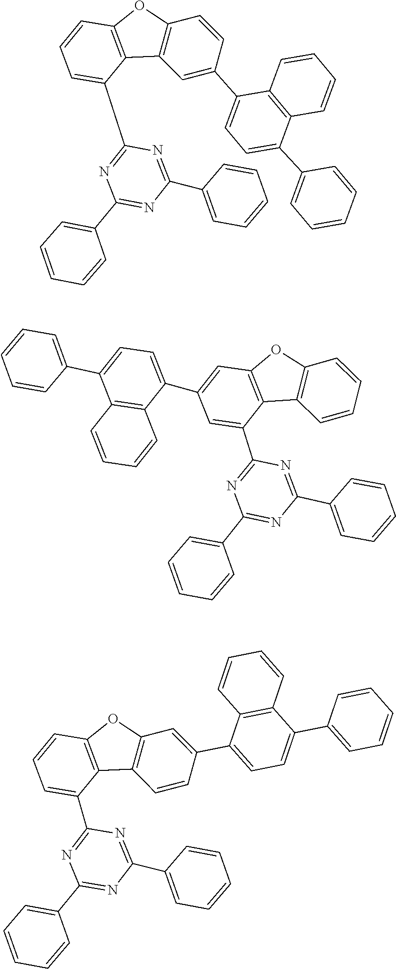 [SMILES: c1ccc(-c2nc(-c3ccccc3)nc(-c3cccc4oc5ccc(-c6ccc(-c7ccccc7)c7ccccc67)cc5c34)n2)cc1 ; c1ccc(-c2nc(-c3ccccc3)nc(-c3cc(-c4ccc(-c5ccccc5)c5ccccc45)cc4oc5ccccc5c34)n2)cc1 ; c1ccc(-c2nc(-c3ccccc3)nc(-c3cccc4oc5cc(-c6ccc(-c7ccccc7)c7ccccc67)ccc5c34)n2)cc1]

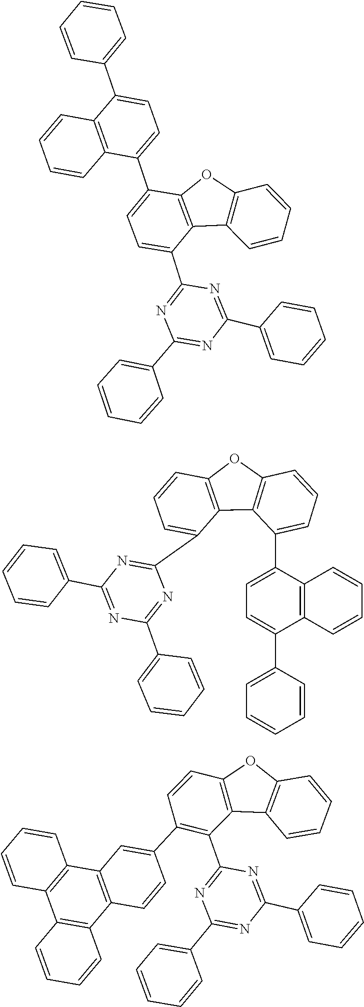

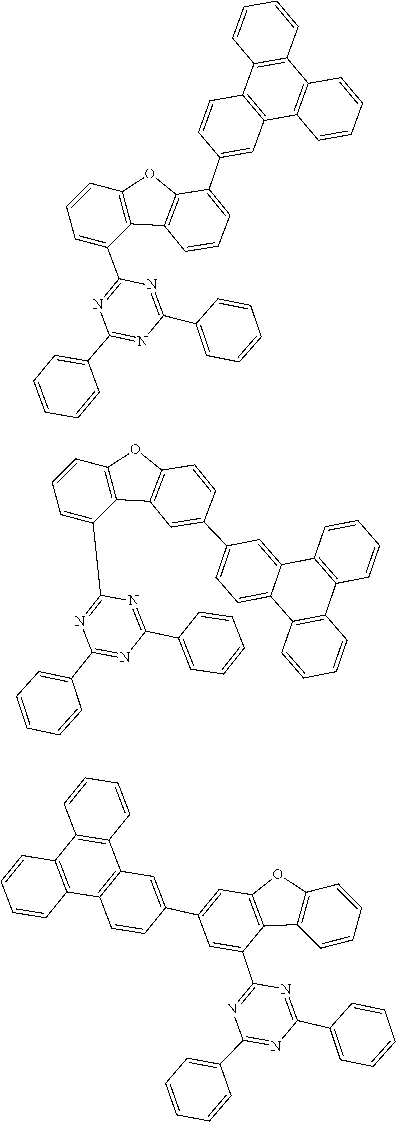

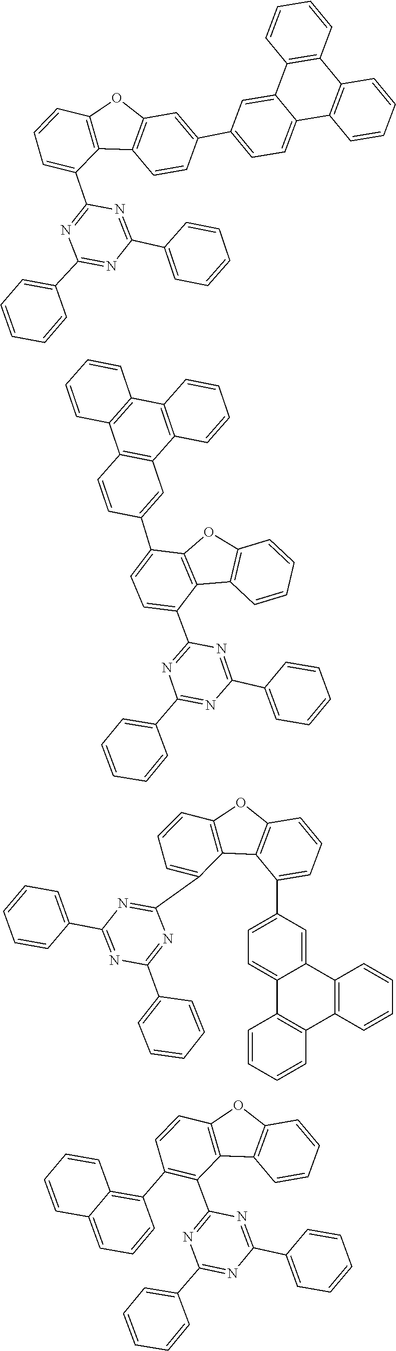

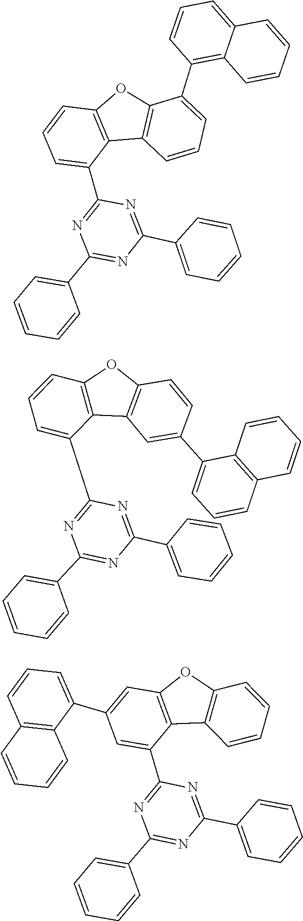

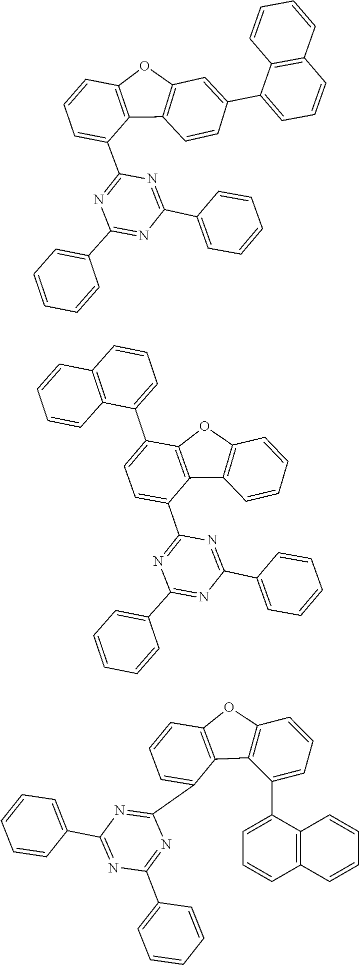 [SMILES: c1ccc(-c2nc(-c3ccccc3)nc(-c3cccc4oc5cc(-c6cccc7ccccc67)ccc5c34)n2)cc1 ; c1ccc(-c2nc(-c3ccccc3)nc(-c3ccc(-c4cccc5ccccc45)c4oc5ccccc5c34)n2)cc1 ; c1ccc(-c2nc(-c3ccccc3)nc(-c3cccc4oc5cccc(-c6cccc7ccccc67)c5c34)n2)cc1]

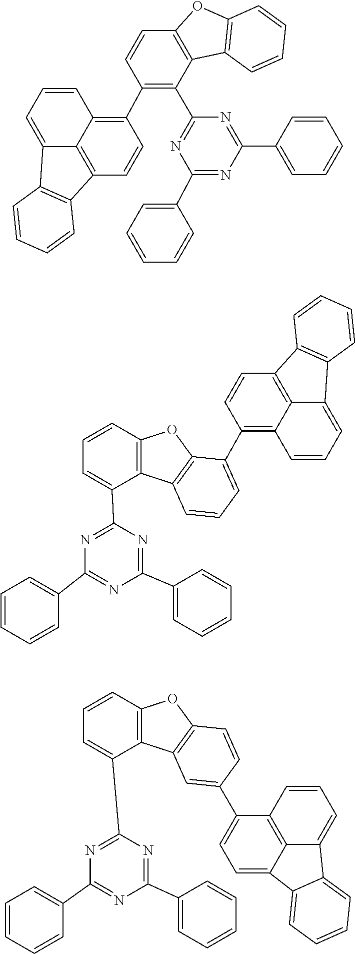 [SMILES: c1ccc(-c2nc(-c3ccccc3)nc(-c3c(-c4ccc5c6c(cccc46)-c4ccccc4-5)ccc4oc5ccccc5c34)n2)cc1 ; c1ccc(-c2nc(-c3ccccc3)nc(-c3cccc4oc5c(-c6ccc7c8c(cccc68)-c6ccccc6-7)cccc5c34)n2)cc1 ; c1ccc(-c2nc(-c3ccccc3)nc(-c3cccc4oc5ccc(-c6ccc7c8c(cccc68)-c6ccccc6-7)cc5c34)n2)cc1]

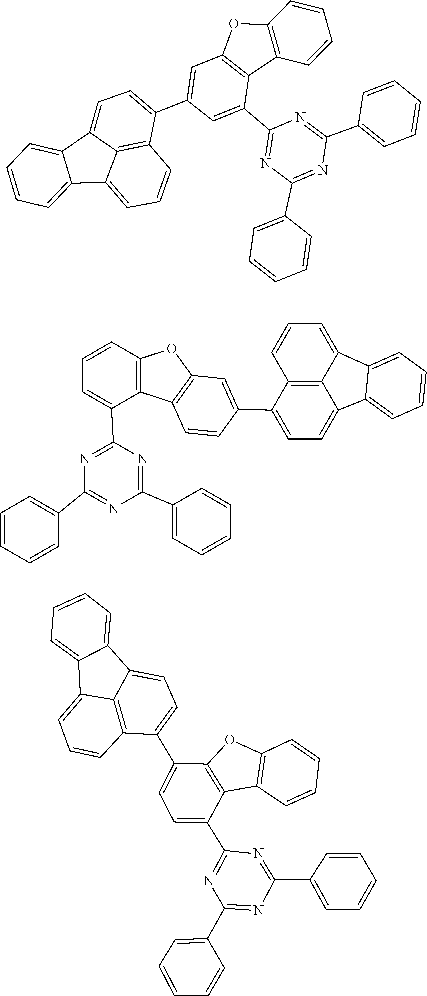

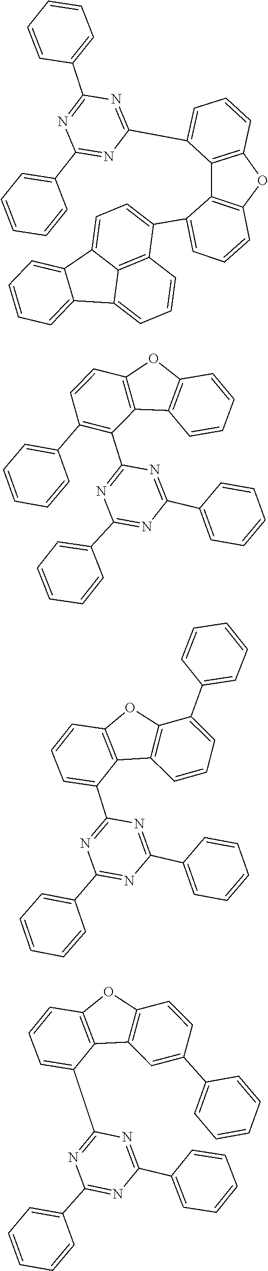

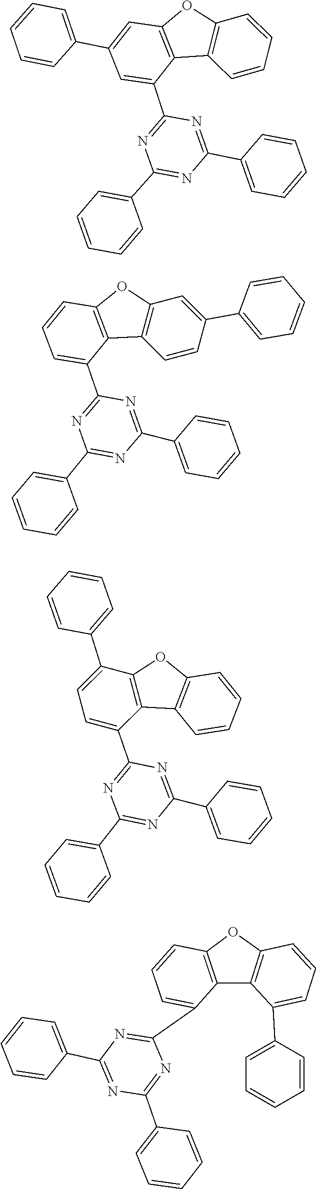

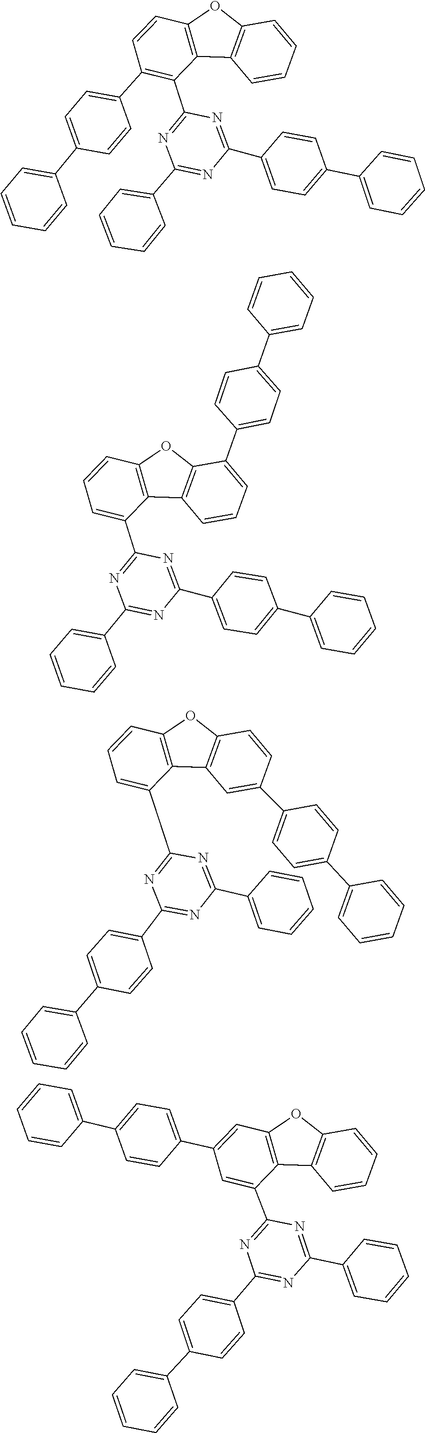

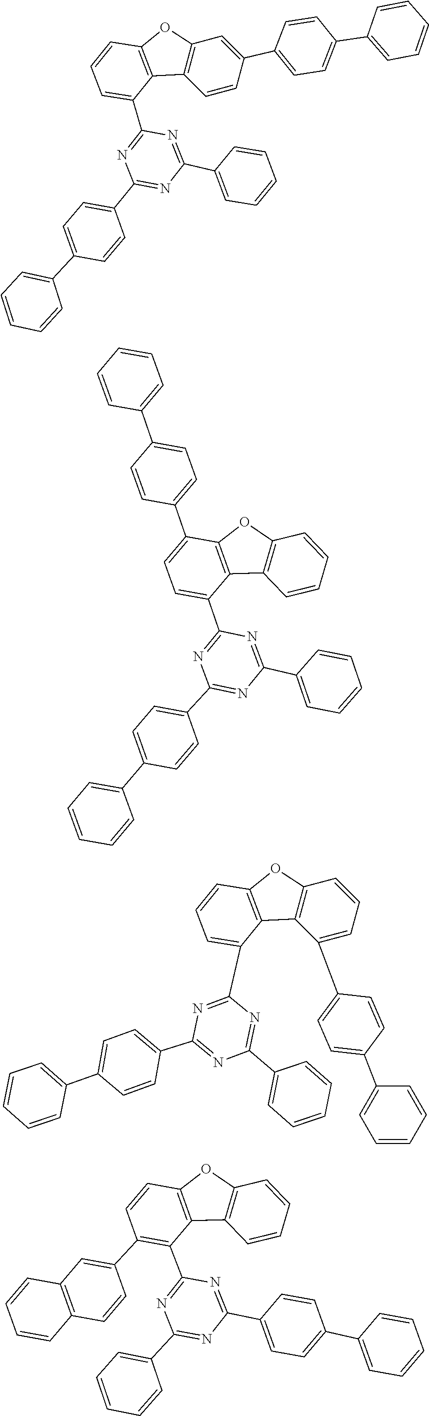 [SMILES: c1ccc(-c2ccc(-c3ccc4c(c3)oc3cccc(-c5nc(-c6ccccc6)nc(-c6ccc(-c7ccccc7)cc6)n5)c34)cc2)cc1 ; c1ccc(-c2ccc(-c3nc(-c4ccccc4)nc(-c4ccc(-c5ccc(-c6ccccc6)cc5)c5oc6ccccc6c45)n3)cc2)cc1 ; c1ccc(-c2ccc(-c3nc(-c4ccccc4)nc(-c4cccc5oc6cccc(-c7ccc(-c8ccccc8)cc7)c6c45)n3)cc2)cc1 ; c1ccc(-c2ccc(-c3nc(-c4ccccc4)nc(-c4c(-c5ccc6ccccc6c5)ccc5oc6ccccc6c45)n3)cc2)cc1]

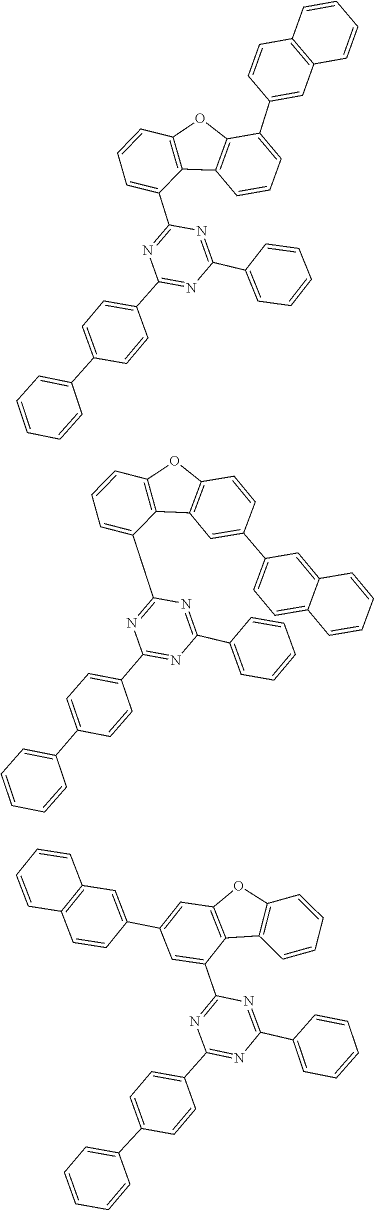

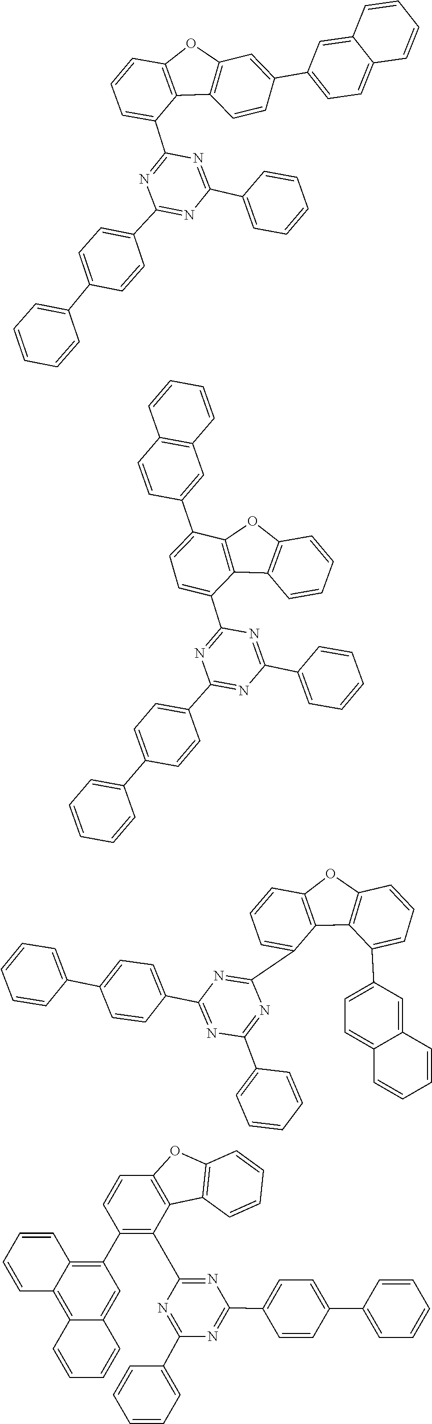

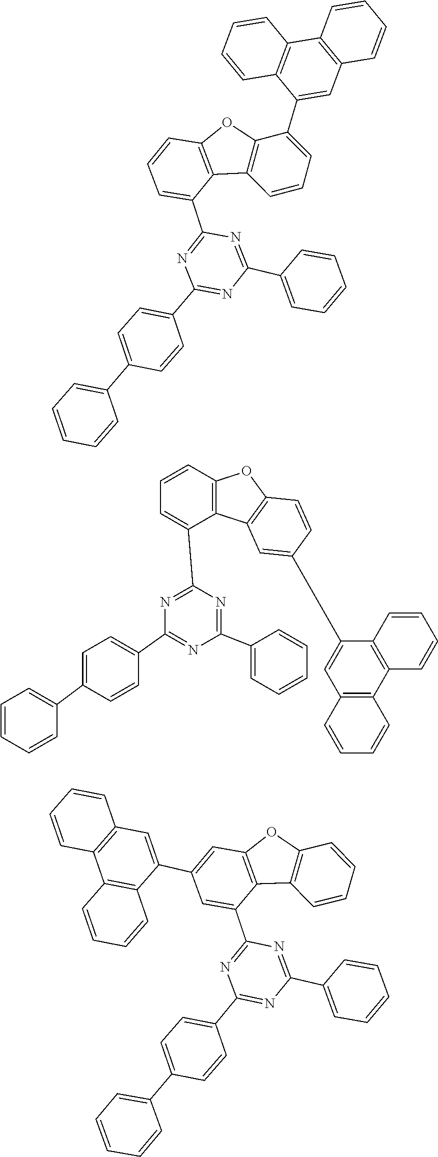 [SMILES: c1ccc(-c2ccc(-c3nc(-c4ccccc4)nc(-c4cccc5oc6c(-c7cc8ccccc8c8ccccc78)cccc6c45)n3)cc2)cc1 ; c1ccc(-c2ccc(-c3nc(-c4ccccc4)nc(-c4cccc5oc6ccc(-c7cc8ccccc8c8ccccc78)cc6c45)n3)cc2)cc1 ; c1ccc(-c2ccc(-c3nc(-c4ccccc4)nc(-c4cc(-c5cc6ccccc6c6ccccc56)cc5oc6ccccc6c45)n3)cc2)cc1]

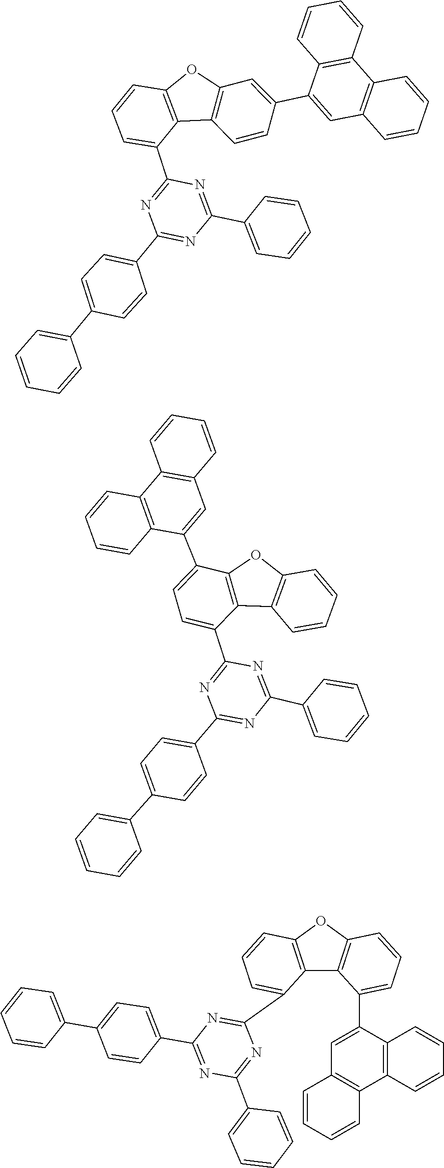 [SMILES: c1ccc(-c2ccc(-c3nc(-c4ccccc4)nc(-c4cccc5oc6cc(-c7cc8ccccc8c8ccccc78)ccc6c45)n3)cc2)cc1 ; c1ccc(-c2ccc(-c3nc(-c4ccccc4)nc(-c4ccc(-c5cc6ccccc6c6ccccc56)c5oc6ccccc6c45)n3)cc2)cc1 ; c1ccc(-c2ccc(-c3nc(-c4ccccc4)nc(-c4cccc5oc6cccc(-c7cc8ccccc8c8ccccc78)c6c45)n3)cc2)cc1]

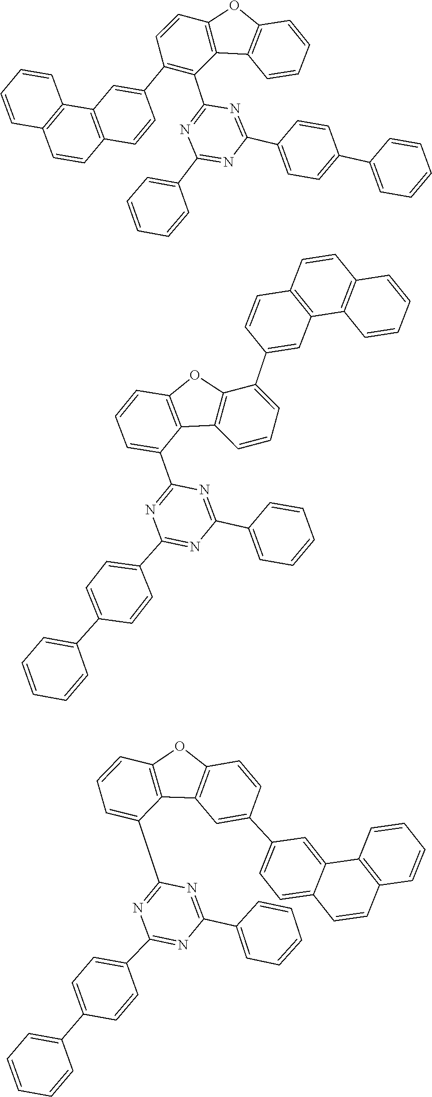 [SMILES: c1ccc(-c2ccc(-c3nc(-c4ccccc4)nc(-c4c(-c5ccc6ccc7ccccc7c6c5)ccc5oc6ccccc6c45)n3)cc2)cc1 ; c1ccc(-c2ccc(-c3nc(-c4ccccc4)nc(-c4cccc5oc6c(-c7ccc8ccc9ccccc9c8c7)cccc6c45)n3)cc2)cc1 ; c1ccc(-c2ccc(-c3nc(-c4ccccc4)nc(-c4cccc5oc6ccc(-c7ccc8ccc9ccccc9c8c7)cc6c45)n3)cc2)cc1]

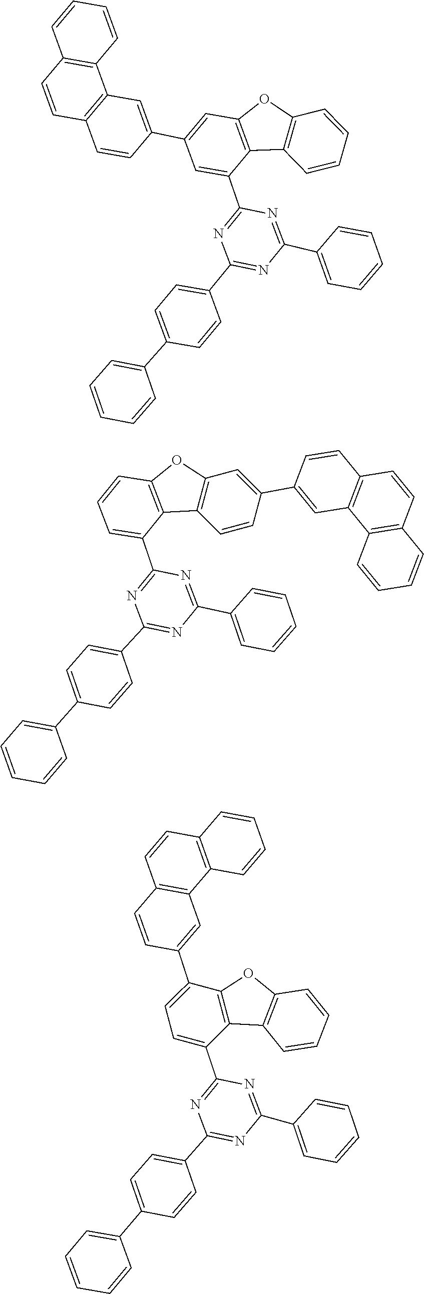 [SMILES: c1ccc(-c2ccc(-c3nc(-c4ccccc4)nc(-c4cc(-c5ccc6ccc7ccccc7c6c5)cc5oc6ccccc6c45)n3)cc2)cc1 ; c1ccc(-c2ccc(-c3nc(-c4ccccc4)nc(-c4cccc5oc6cc(-c7ccc8ccc9ccccc9c8c7)ccc6c45)n3)cc2)cc1 ; c1ccc(-c2ccc(-c3nc(-c4ccccc4)nc(-c4ccc(-c5ccc6ccc7ccccc7c6c5)c5oc6ccccc6c45)n3)cc2)cc1]

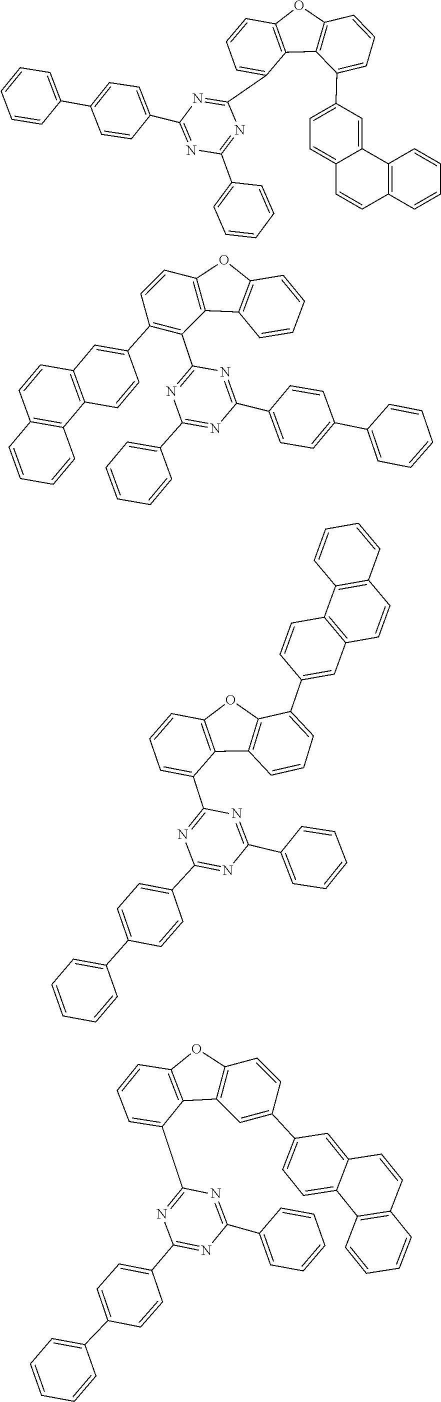

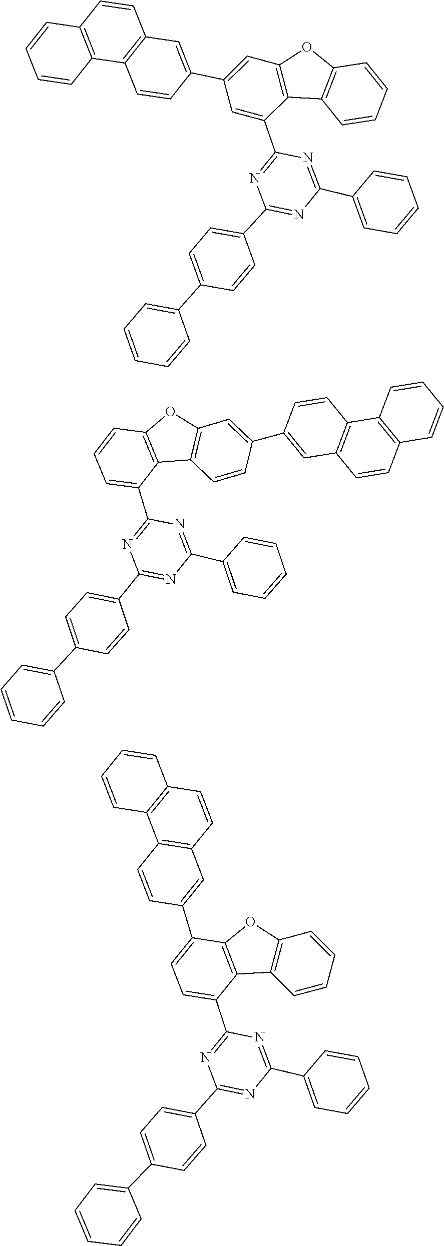

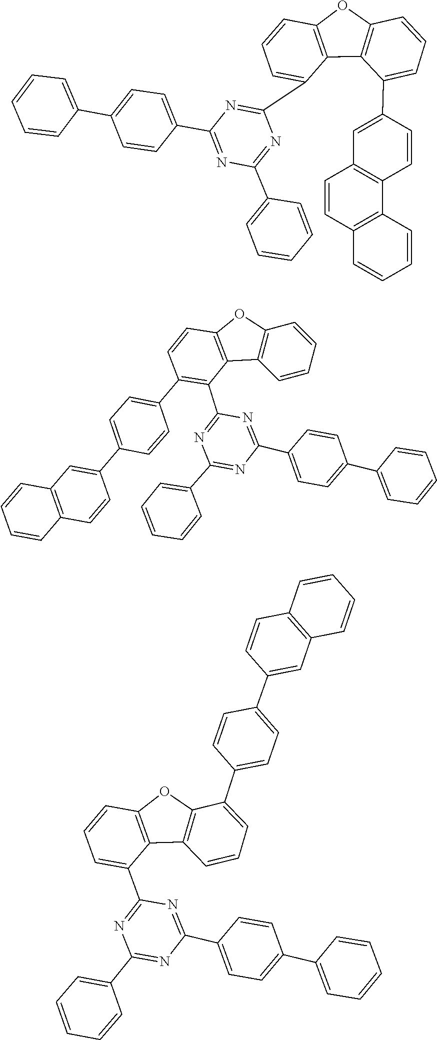 [SMILES: c1ccc(-c2ccc(-c3nc(-c4ccccc4)nc(-c4cccc5oc6cccc(-c7ccc8c(ccc9ccccc98)c7)c6c45)n3)cc2)cc1 ; c1ccc(-c2ccc(-c3nc(-c4ccccc4)nc(-c4c(-c5ccc(-c6ccc7ccccc7c6)cc5)ccc5oc6ccccc6c45)n3)cc2)cc1 ; c1ccc(-c2ccc(-c3nc(-c4ccccc4)nc(-c4cccc5oc6c(-c7ccc(-c8ccc9ccccc9c8)cc7)cccc6c45)n3)cc2)cc1]

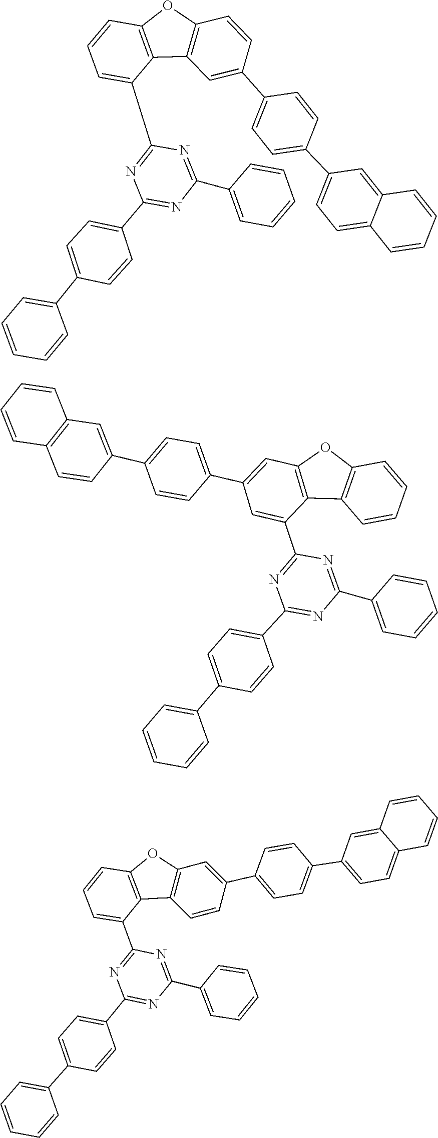

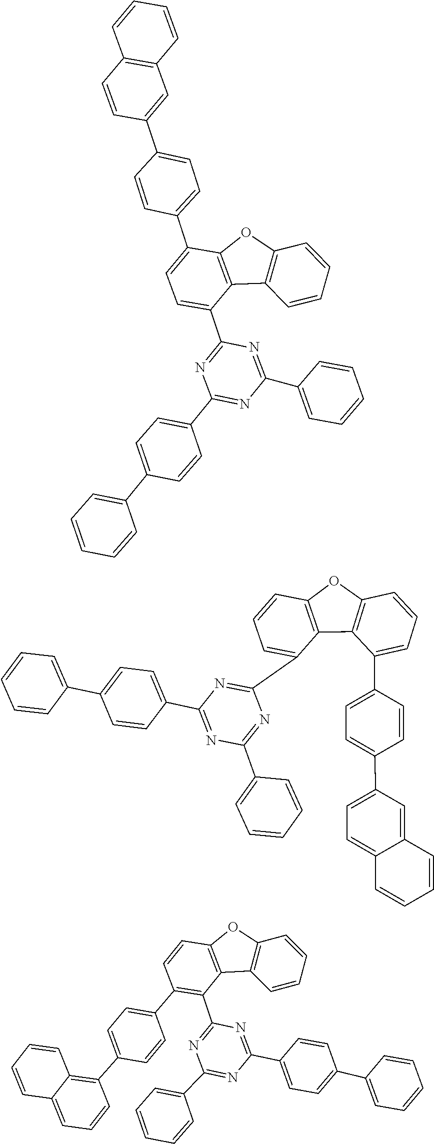 [SMILES: c1ccc(-c2ccc(-c3nc(-c4ccccc4)nc(-c4ccc(-c5ccc(-c6ccc7ccccc7c6)cc5)c5oc6ccccc6c45)n3)cc2)cc1 ; c1ccc(-c2ccc(-c3nc(-c4ccccc4)nc(-c4cccc5oc6cccc(-c7ccc(-c8ccc9ccccc9c8)cc7)c6c45)n3)cc2)cc1 ; c1ccc(-c2ccc(-c3nc(-c4ccccc4)nc(-c4c(-c5ccc(-c6cccc7ccccc67)cc5)ccc5oc6ccccc6c45)n3)cc2)cc1]

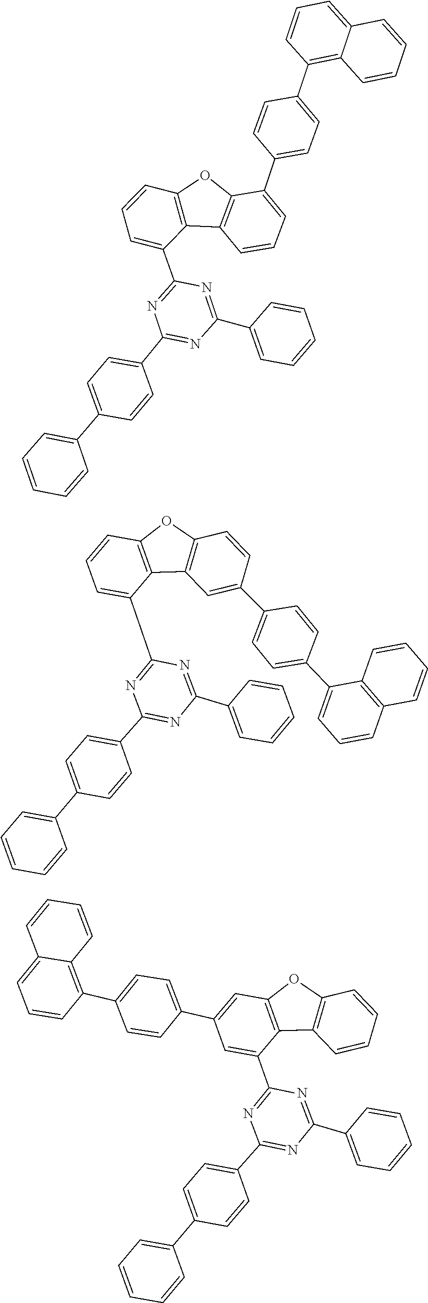

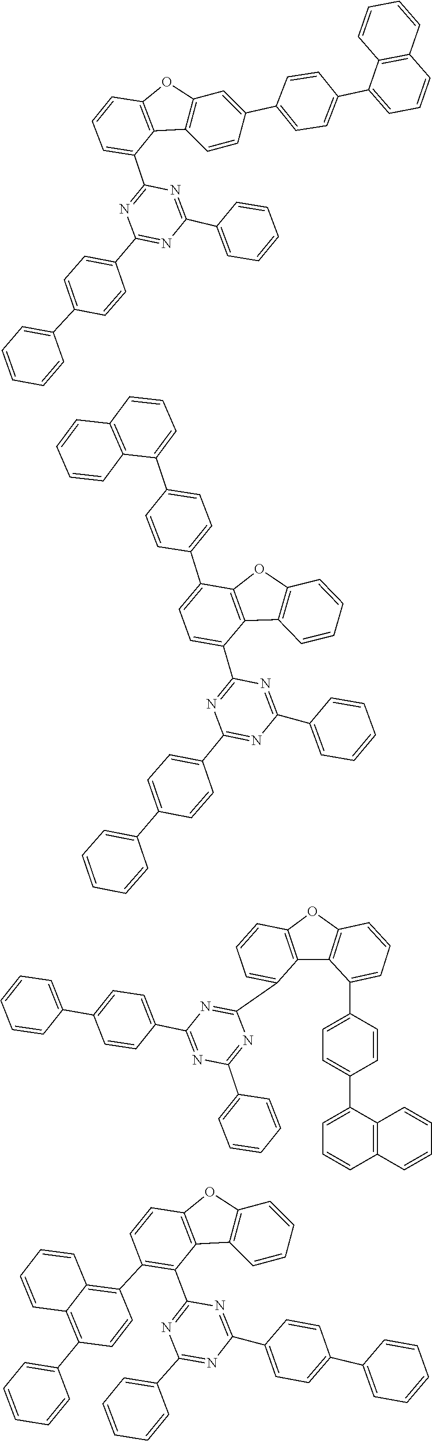 [SMILES: c1ccc(-c2ccc(-c3nc(-c4ccccc4)nc(-c4cccc5oc6cc(-c7ccc(-c8cccc9ccccc89)cc7)ccc6c45)n3)cc2)cc1 ; c1ccc(-c2ccc(-c3nc(-c4ccccc4)nc(-c4ccc(-c5ccc(-c6cccc7ccccc67)cc5)c5oc6ccccc6c45)n3)cc2)cc1 ; c1ccc(-c2ccc(-c3nc(-c4ccccc4)nc(-c4cccc5oc6cccc(-c7ccc(-c8cccc9ccccc89)cc7)c6c45)n3)cc2)cc1 ; c1ccc(-c2ccc(-c3nc(-c4ccccc4)nc(-c4c(-c5ccc(-c6ccccc6)c6ccccc56)ccc5oc6ccccc6c45)n3)cc2)cc1]

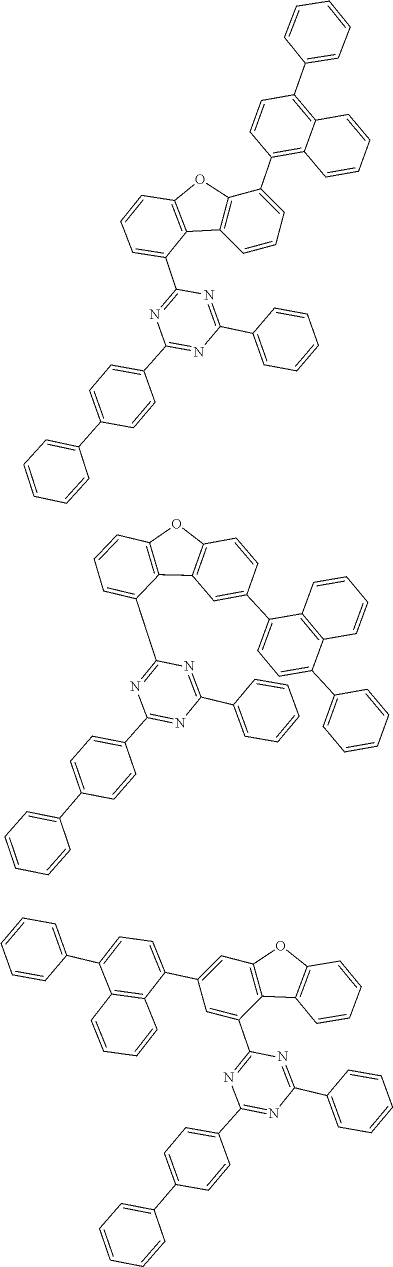 [SMILES: c1ccc(-c2ccc(-c3nc(-c4ccccc4)nc(-c4cccc5oc6c(-c7ccc(-c8ccccc8)c8ccccc78)cccc6c45)n3)cc2)cc1 ; c1ccc(-c2ccc(-c3nc(-c4ccccc4)nc(-c4cccc5oc6ccc(-c7ccc(-c8ccccc8)c8ccccc78)cc6c45)n3)cc2)cc1 ; c1ccc(-c2ccc(-c3nc(-c4ccccc4)nc(-c4cc(-c5ccc(-c6ccccc6)c6ccccc56)cc5oc6ccccc6c45)n3)cc2)cc1]

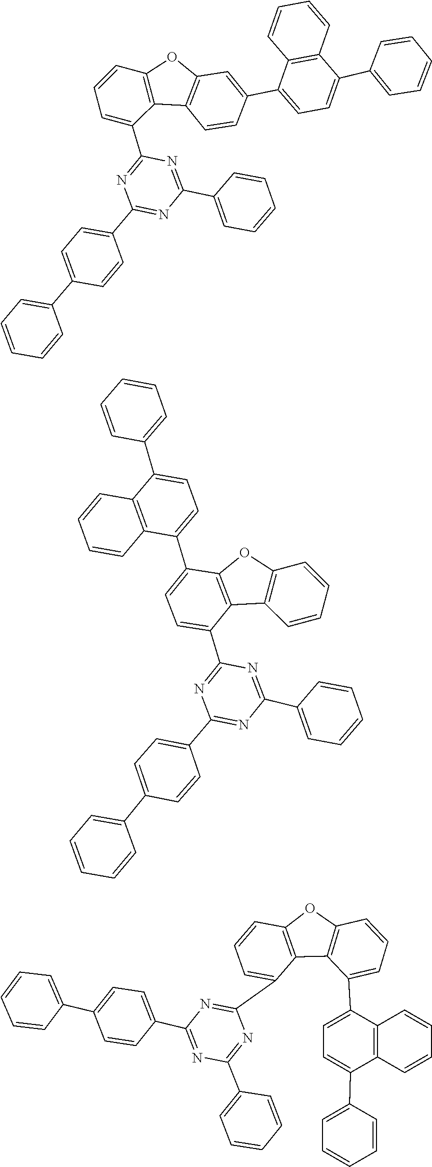 [SMILES: c1ccc(-c2ccc(-c3nc(-c4ccccc4)nc(-c4cccc5oc6cc(-c7ccc(-c8ccccc8)c8ccccc78)ccc6c45)n3)cc2)cc1 ; c1ccc(-c2ccc(-c3nc(-c4ccccc4)nc(-c4ccc(-c5ccc(-c6ccccc6)c6ccccc56)c5oc6ccccc6c45)n3)cc2)cc1 ; c1ccc(-c2ccc(-c3nc(-c4ccccc4)nc(-c4cccc5oc6cccc(-c7ccc(-c8ccccc8)c8ccccc78)c6c45)n3)cc2)cc1]

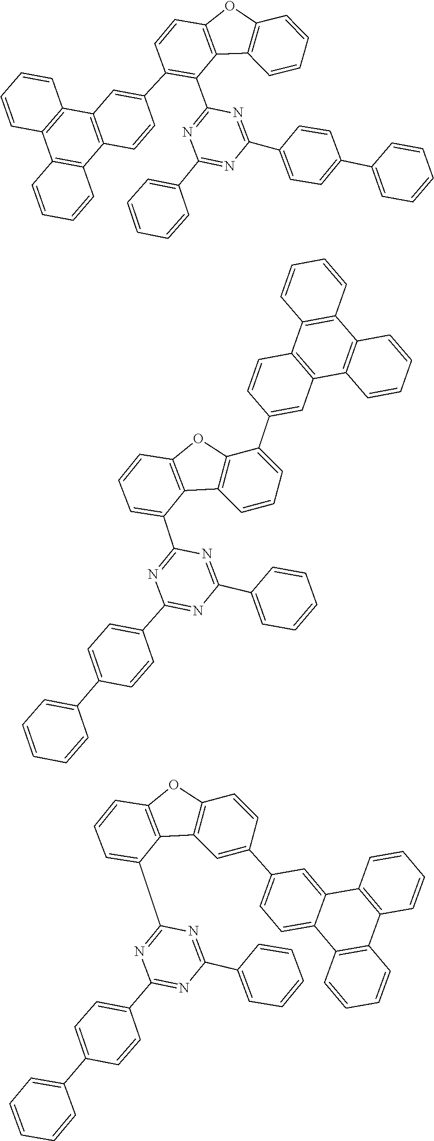 [SMILES: c1ccc(-c2ccc(-c3nc(-c4ccccc4)nc(-c4c(-c5ccc6c7ccccc7c7ccccc7c6c5)ccc5oc6ccccc6c45)n3)cc2)cc1 ; c1ccc(-c2ccc(-c3nc(-c4ccccc4)nc(-c4cccc5oc6c(-c7ccc8c9ccccc9c9ccccc9c8c7)cccc6c45)n3)cc2)cc1 ; c1ccc(-c2ccc(-c3nc(-c4ccccc4)nc(-c4cccc5oc6ccc(-c7ccc8c9ccccc9c9ccccc9c8c7)cc6c45)n3)cc2)cc1]

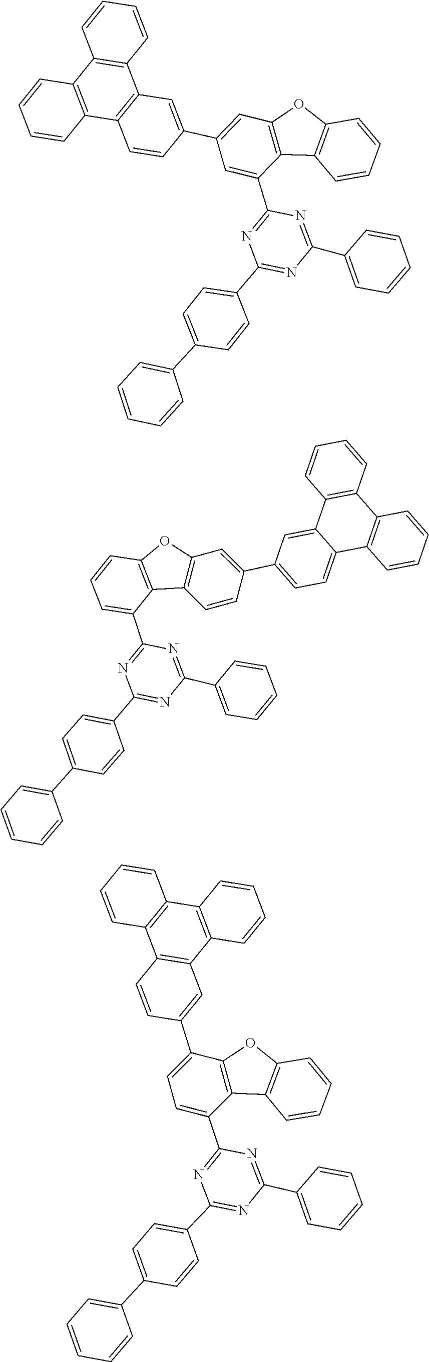 [SMILES: c1ccc(-c2ccc(-c3nc(-c4ccccc4)nc(-c4cc(-c5ccc6c7ccccc7c7ccccc7c6c5)cc5oc6ccccc6c45)n3)cc2)cc1 ; c1ccc(-c2ccc(-c3nc(-c4ccccc4)nc(-c4cccc5oc6cc(-c7ccc8c9ccccc9c9ccccc9c8c7)ccc6c45)n3)cc2)cc1 ; c1ccc(-c2ccc(-c3nc(-c4ccccc4)nc(-c4ccc(-c5ccc6c7ccccc7c7ccccc7c6c5)c5oc6ccccc6c45)n3)cc2)cc1]

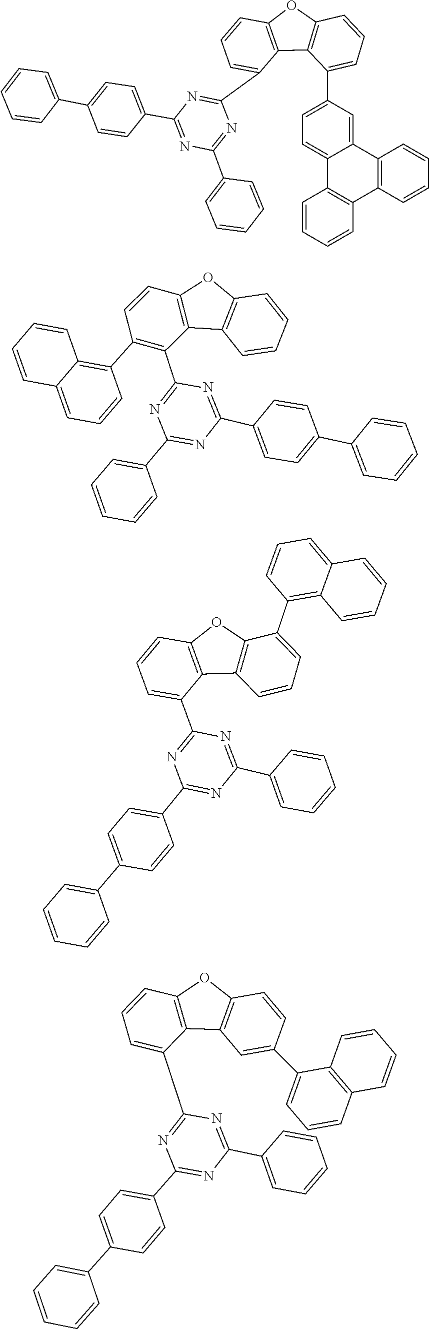 [SMILES: c1ccc(-c2ccc(-c3nc(-c4ccccc4)nc(-c4cccc5oc6cccc(-c7ccc8c9ccccc9c9ccccc9c8c7)c6c45)n3)cc2)cc1 ; c1ccc(-c2ccc(-c3nc(-c4ccccc4)nc(-c4c(-c5cccc6ccccc56)ccc5oc6ccccc6c45)n3)cc2)cc1 ; c1ccc(-c2ccc(-c3nc(-c4ccccc4)nc(-c4cccc5oc6c(-c7cccc8ccccc78)cccc6c45)n3)cc2)cc1 ; c1ccc(-c2ccc(-c3nc(-c4ccccc4)nc(-c4cccc5oc6ccc(-c7cccc8ccccc78)cc6c45)n3)cc2)cc1]

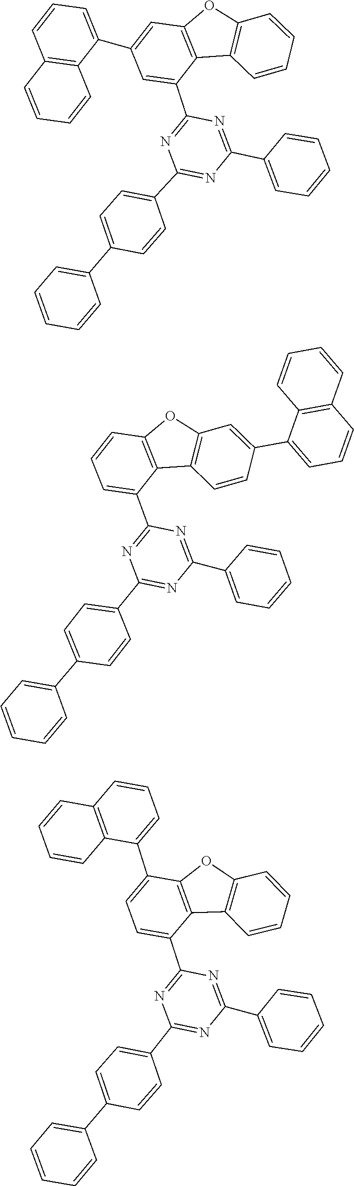 [SMILES: c1ccc(-c2ccc(-c3nc(-c4ccccc4)nc(-c4cc(-c5cccc6ccccc56)cc5oc6ccccc6c45)n3)cc2)cc1 ; c1ccc(-c2ccc(-c3nc(-c4ccccc4)nc(-c4cccc5oc6cc(-c7cccc8ccccc78)ccc6c45)n3)cc2)cc1 ; c1ccc(-c2ccc(-c3nc(-c4ccccc4)nc(-c4ccc(-c5cccc6ccccc56)c5oc6ccccc6c45)n3)cc2)cc1]

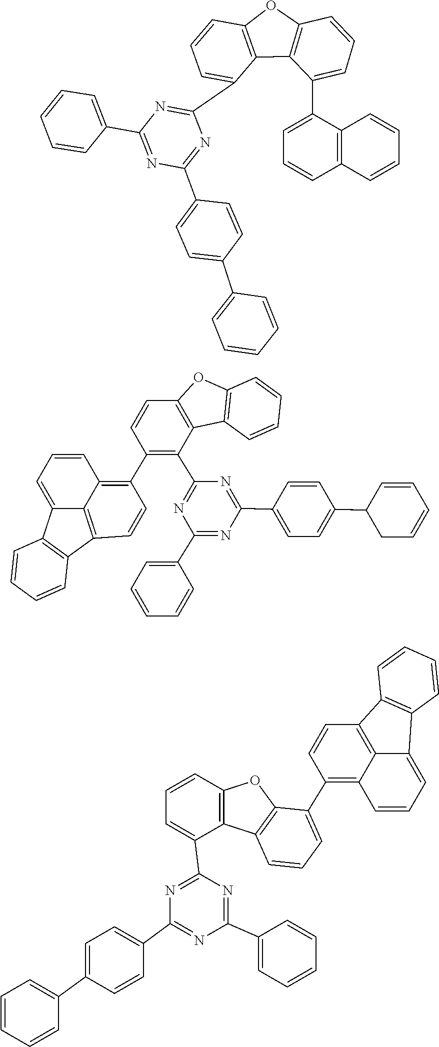

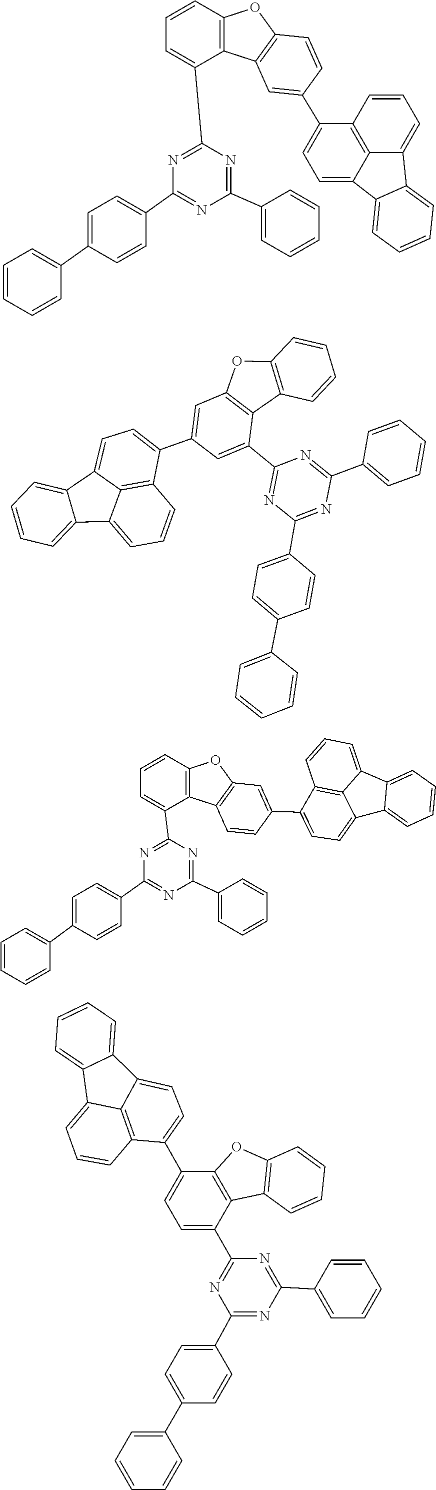

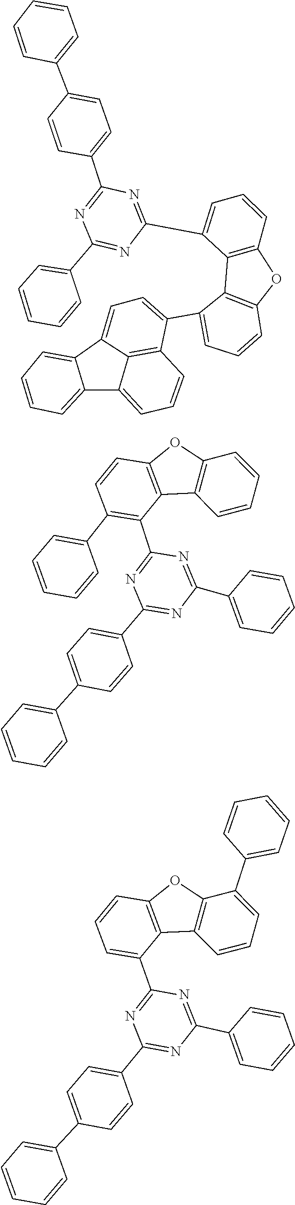

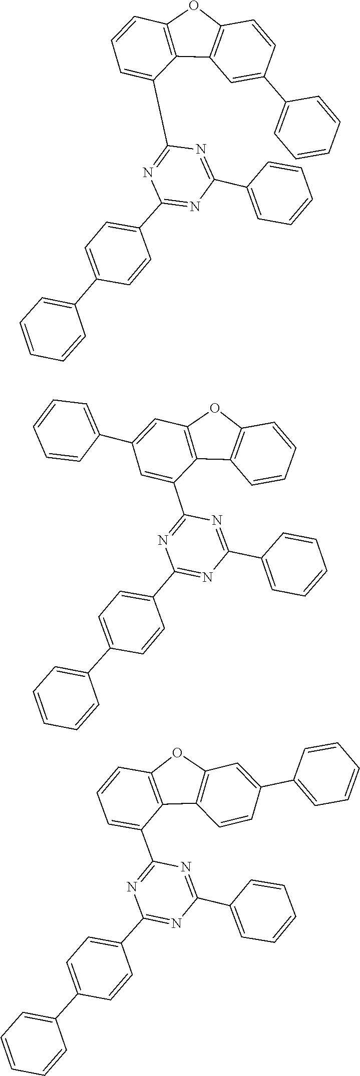

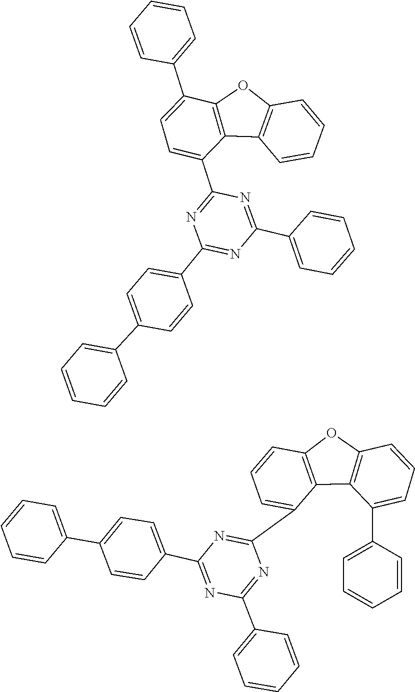 [SMILES: c1ccc(-c2ccc(-c3nc(-c4ccccc4)nc(-c4ccc(-c5ccccc5)c5oc6ccccc6c45)n3)cc2)cc1 ; c1ccc(-c2ccc(-c3nc(-c4ccccc4)nc(-c4cccc5oc6cccc(-c7ccccc7)c6c45)n3)cc2)cc1]

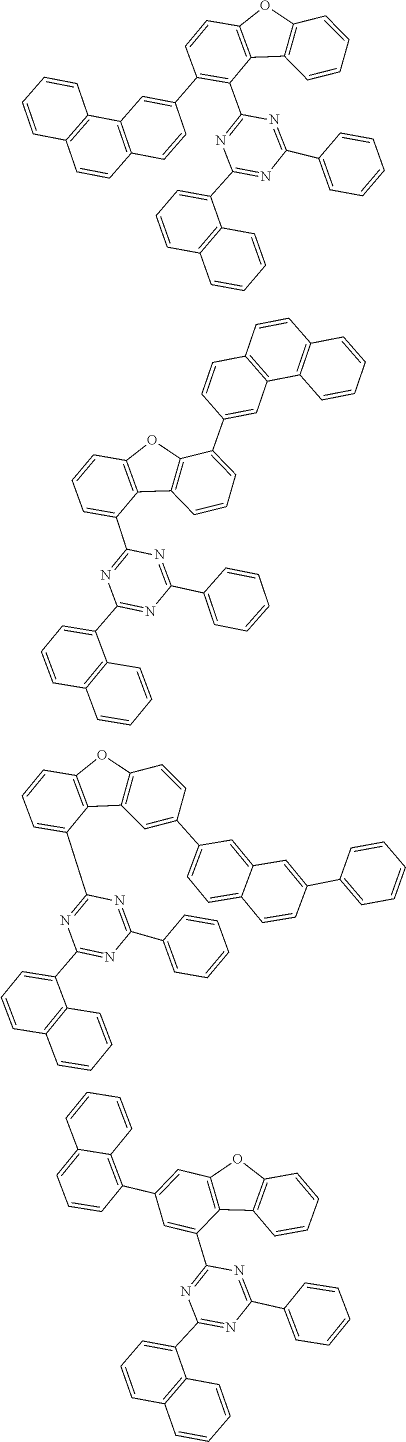

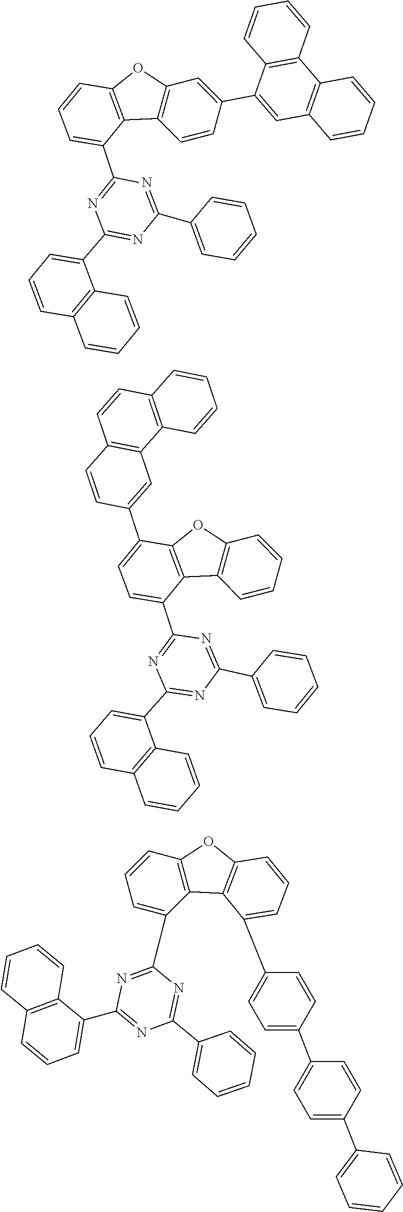

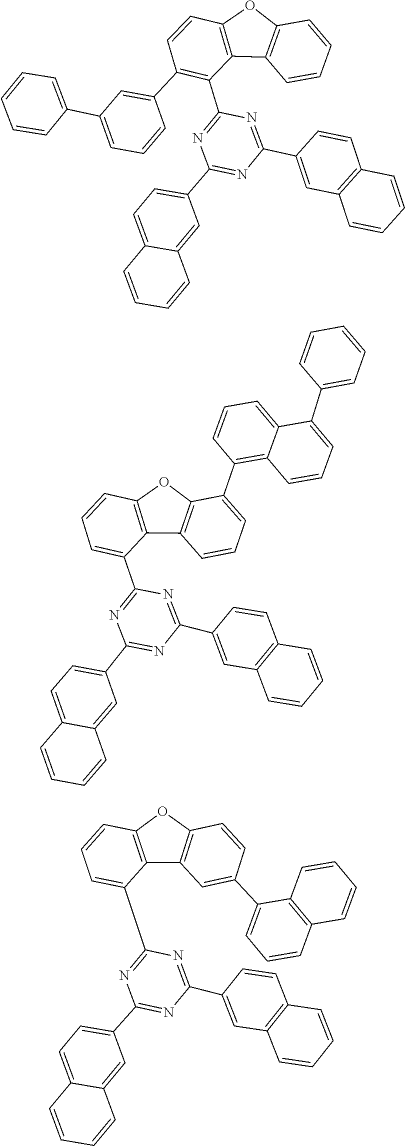

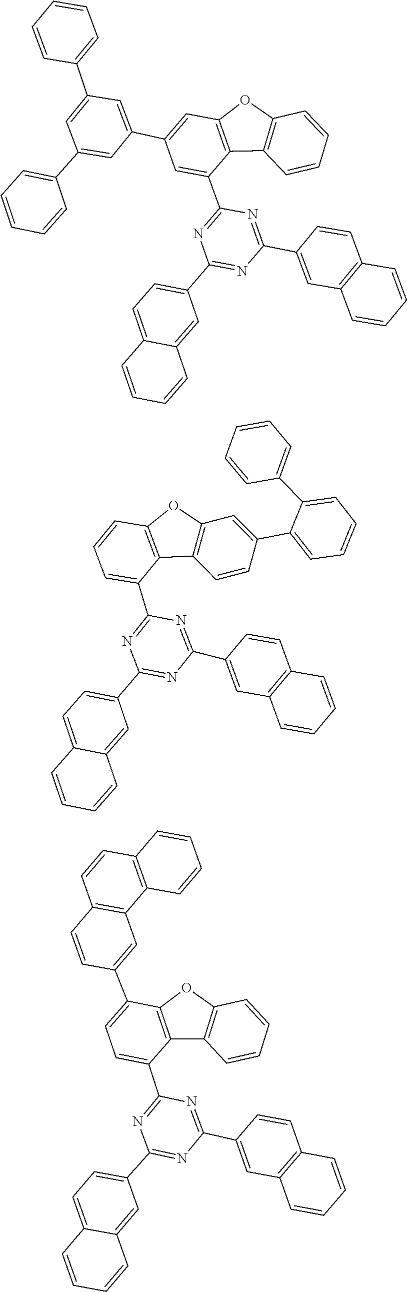

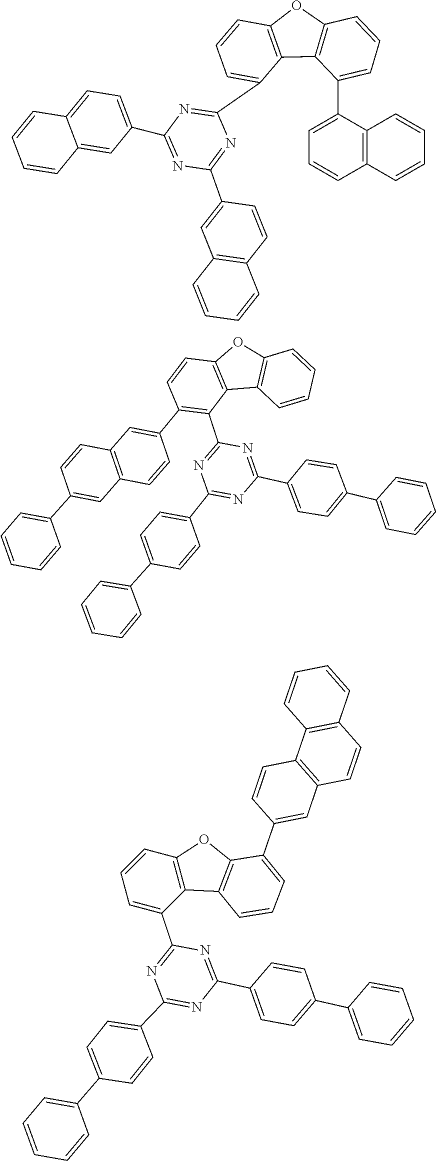

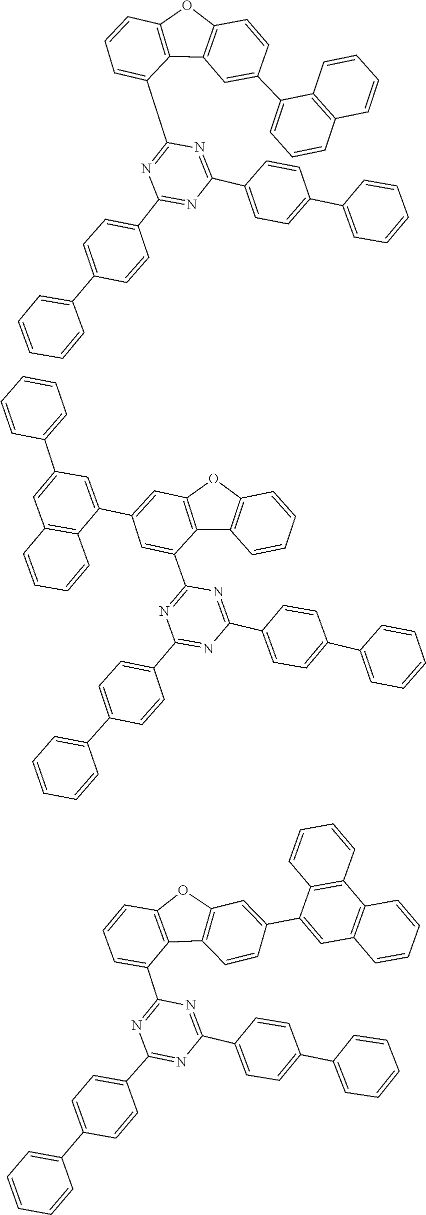

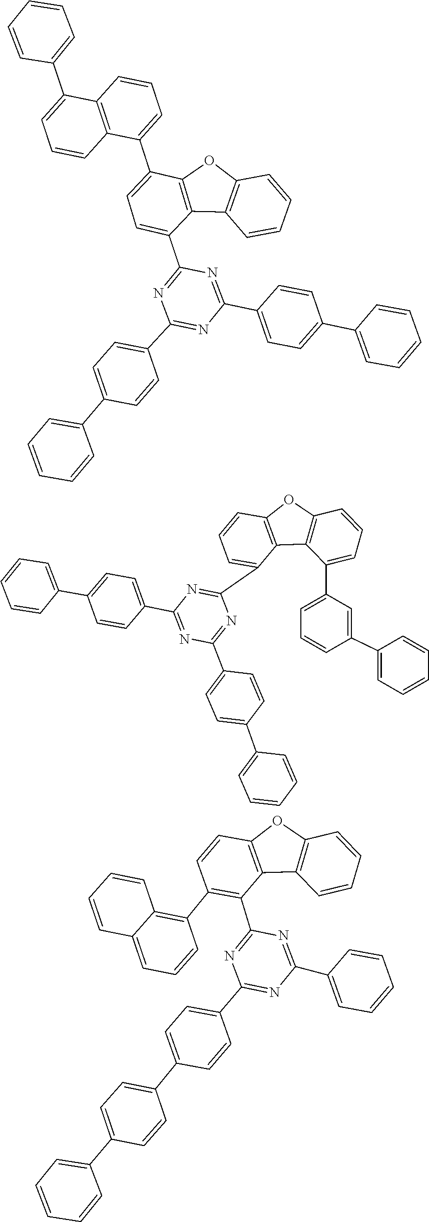

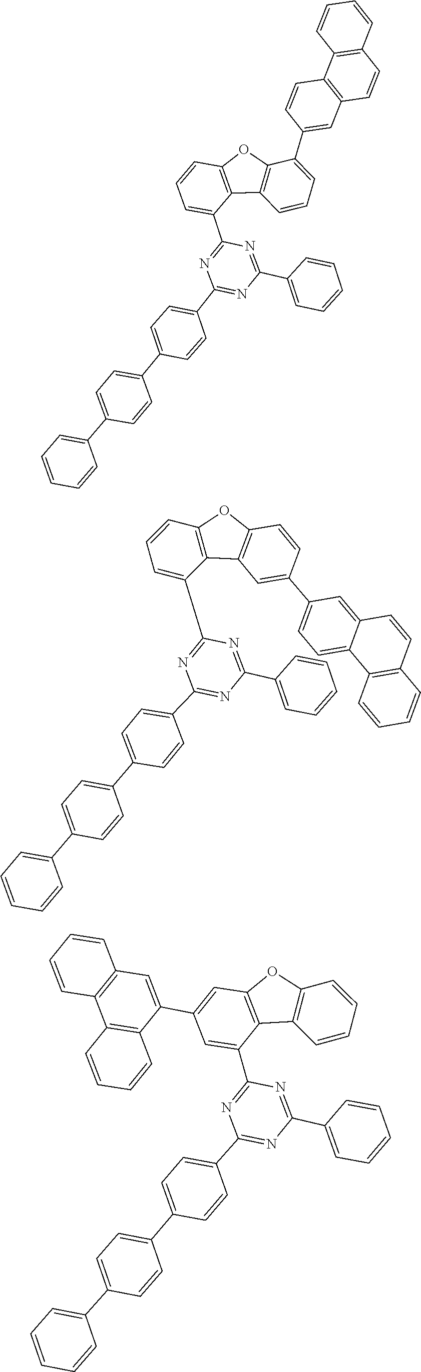

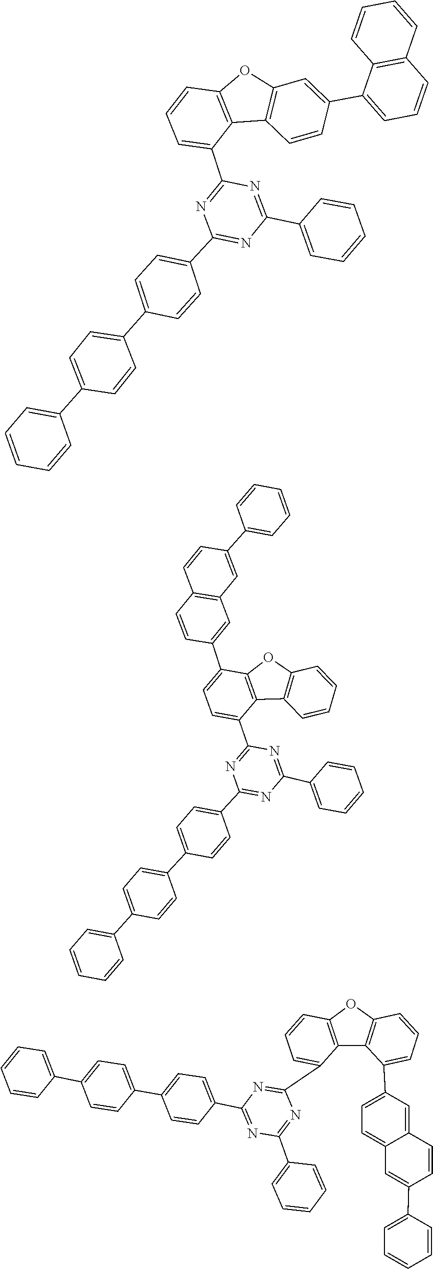 [SMILES: c1ccc(-c2ccc(-c3ccc(-c4nc(-c5ccccc5)nc(-c5cccc6oc7cc(-c8cccc9ccccc89)ccc7c56)n4)cc3)cc2)cc1 ; c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9cc(-c%10ccccc%10)ccc9c8)c7c56)n4)cc3)cc2)cc1]

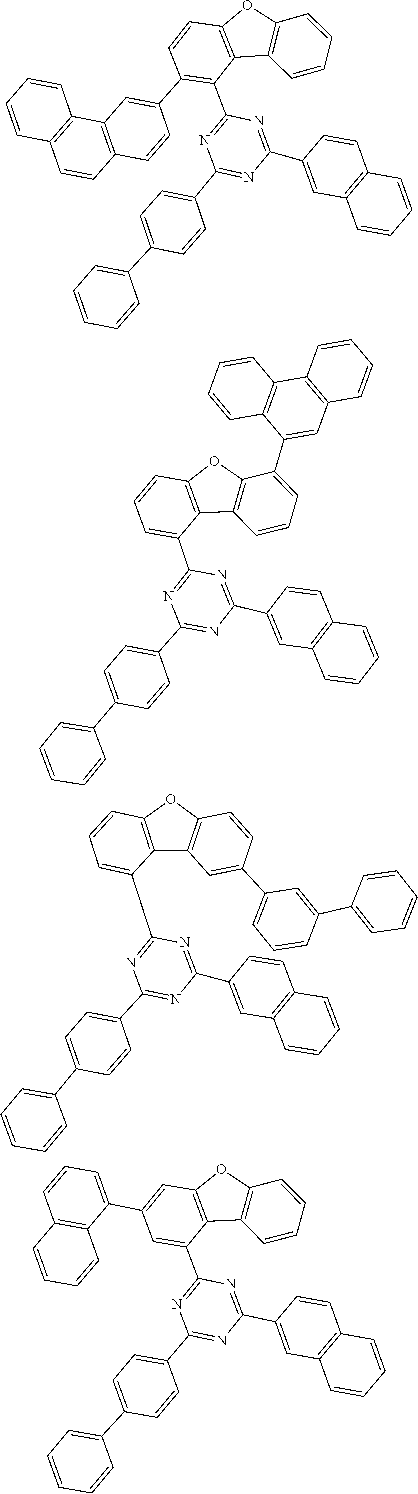

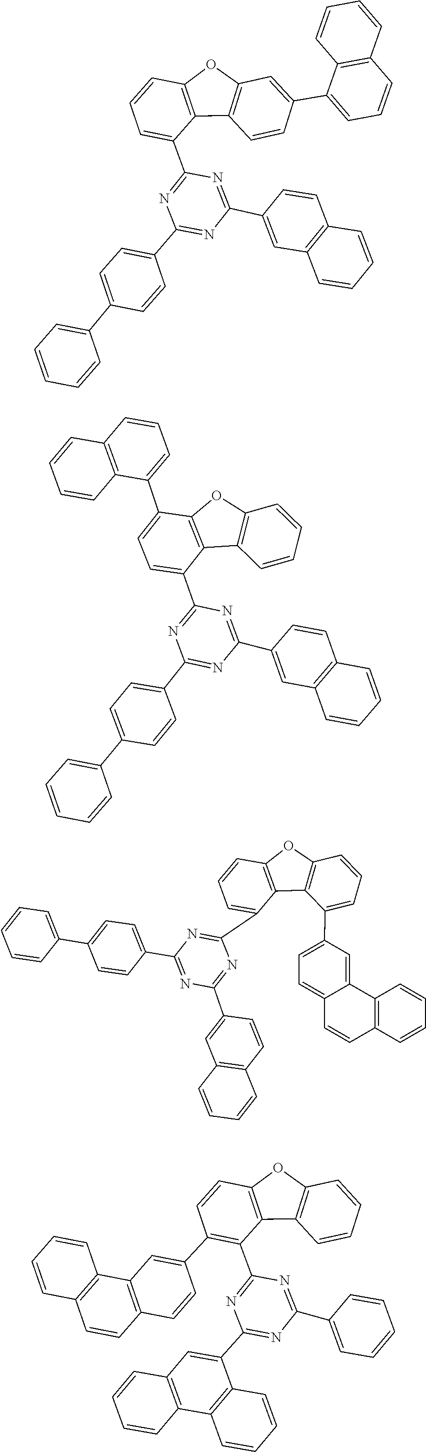 [SMILES: c1ccc(-c2ccc(-c3nc(-c4ccc5ccccc5c4)nc(-c4cccc5oc6cc(-c7cccc8ccccc78)ccc6c45)n3)cc2)cc1 ; c1ccc(-c2ccc(-c3nc(-c4ccc5ccccc5c4)nc(-c4ccc(-c5cccc6ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8ccc9ccccc9c8c7)c6c45)n3)cc2)cc1 ; c1ccc(-c2nc(-c3cc4ccccc4c4ccccc34)nc(-c3c(-c4ccc5ccc6ccccc6c5c4)ccc4oc5ccccc5c34)n2)cc1]

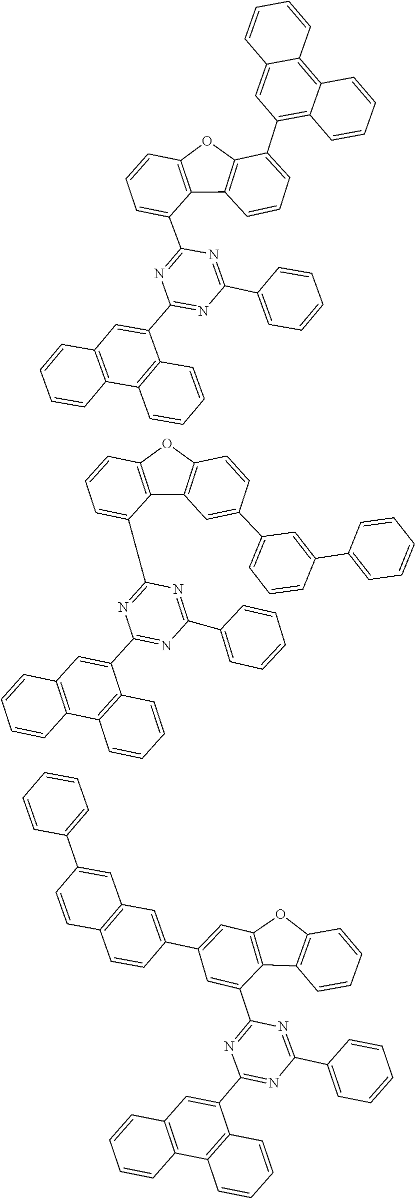

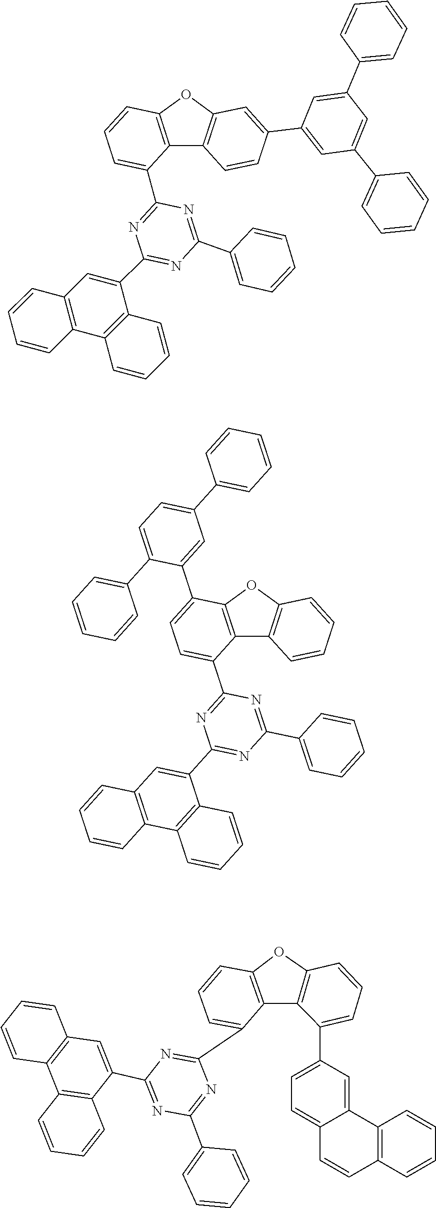 [SMILES: c1ccc(-c2cc(-c3ccccc3)cc(-c3ccc4c(c3)oc3cccc(-c5nc(-c6ccccc6)nc(-c6cc7ccccc7c7ccccc67)n5)c34)c2)cc1 ; c1ccc(-c2ccc(-c3ccccc3)c(-c3ccc(-c4nc(-c5ccccc5)nc(-c5cc6ccccc6c6ccccc56)n4)c4c3oc3ccccc34)c2)cc1 ; c1ccc(-c2nc(-c3cc4ccccc4c4ccccc34)nc(-c3cccc4oc5cccc(-c6ccc7ccc8ccccc8c7c6)c5c34)n2)cc1]

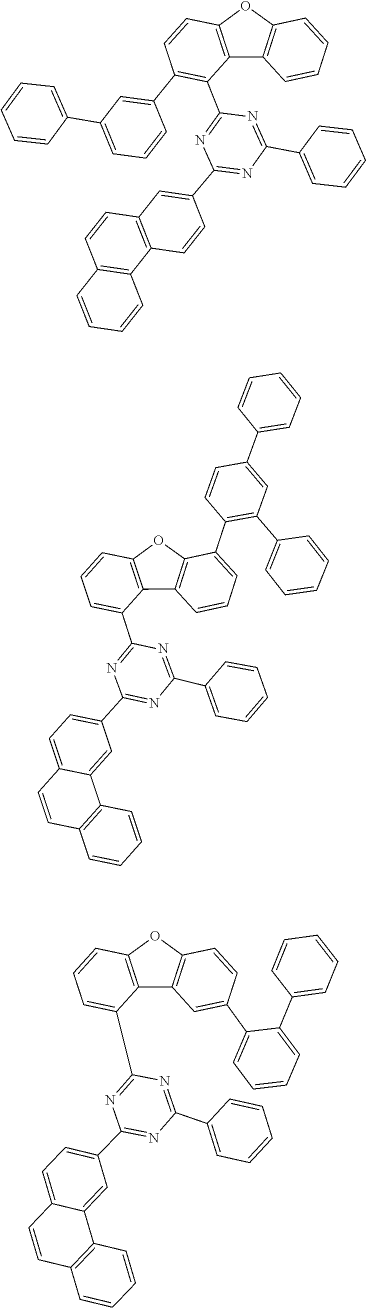 [SMILES: c1ccc(-c2cccc(-c3ccc4oc5ccccc5c4c3-c3nc(-c4ccccc4)nc(-c4ccc5c(ccc6ccccc65)c4)n3)c2)cc1 ; c1ccc(-c2ccc(-c3cccc4c3oc3cccc(-c5nc(-c6ccccc6)nc(-c6ccc7ccc8ccccc8c7c6)n5)c34)c(-c3ccccc3)c2)cc1 ; c1ccc(-c2nc(-c3ccc4ccc5ccccc5c4c3)nc(-c3cccc4oc5ccc(-c6ccccc6-c6ccccc6)cc5c34)n2)cc1]

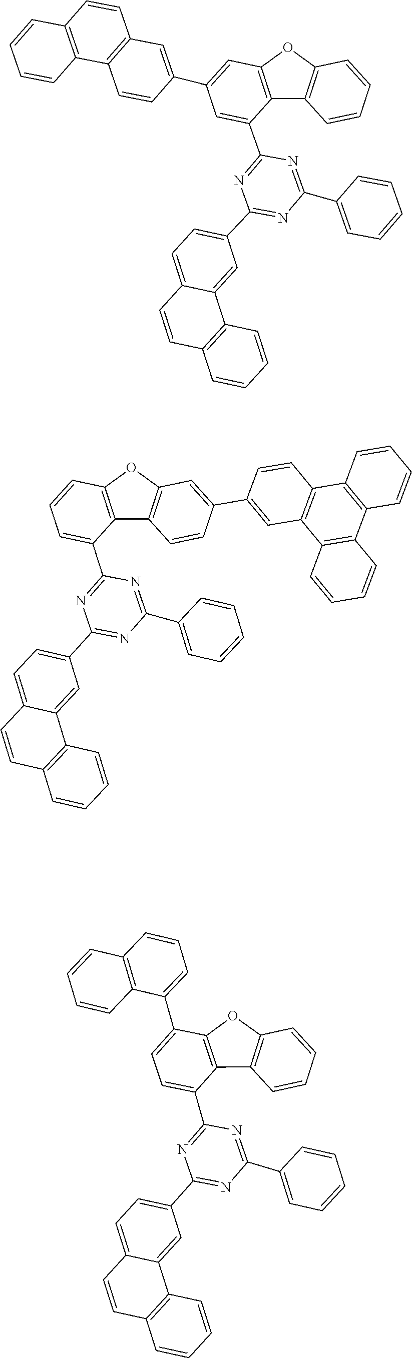 [SMILES: c1ccc(-c2nc(-c3ccc4ccc5ccccc5c4c3)nc(-c3cc(-c4ccc5c(ccc6ccccc65)c4)cc4oc5ccccc5c34)n2)cc1 ; c1ccc(-c2nc(-c3ccc4ccc5ccccc5c4c3)nc(-c3cccc4oc5cc(-c6ccc7c8ccccc8c8ccccc8c7c6)ccc5c34)n2)cc1 ; c1ccc(-c2nc(-c3ccc4ccc5ccccc5c4c3)nc(-c3ccc(-c4cccc5ccccc45)c4oc5ccccc5c34)n2)cc1]

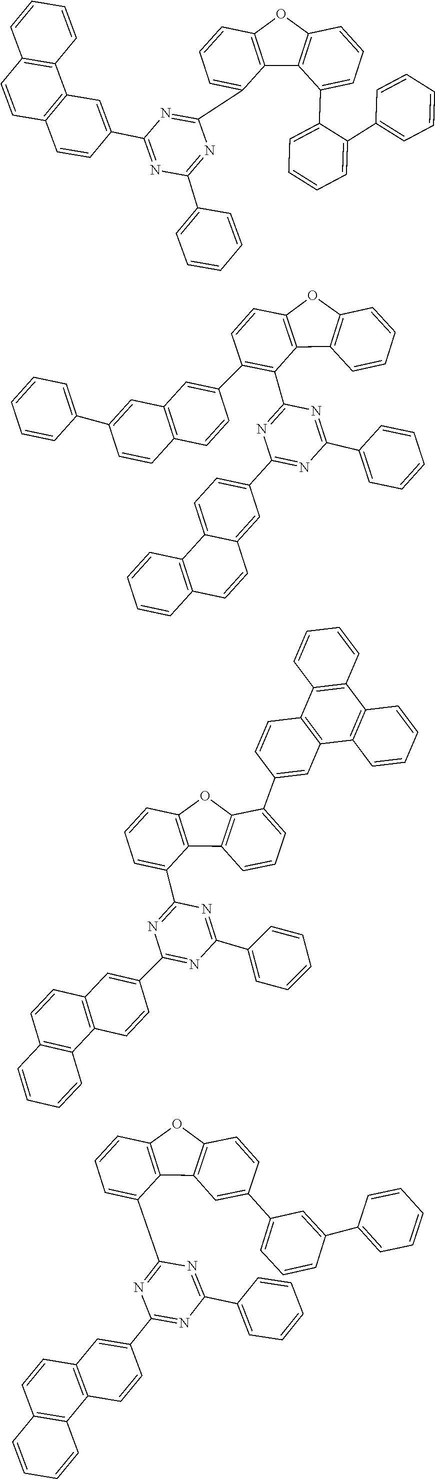 [SMILES: c1ccc(-c2nc(-c3ccc4ccc5ccccc5c4c3)nc(-c3cccc4oc5cccc(-c6ccccc6-c6ccccc6)c5c34)n2)cc1 ; c1ccc(-c2ccc3ccc(-c4ccc5oc6ccccc6c5c4-c4nc(-c5ccccc5)nc(-c5ccc6c(ccc7ccccc76)c5)n4)cc3c2)cc1 ; c1ccc(-c2nc(-c3ccc4c(ccc5ccccc54)c3)nc(-c3cccc4oc5c(-c6ccc7c8ccccc8c8ccccc8c7c6)cccc5c34)n2)cc1 ; c1ccc(-c2cccc(-c3ccc4oc5cccc(-c6nc(-c7ccccc7)nc(-c7ccc8c(ccc9ccccc98)c7)n6)c5c4c3)c2)cc1]

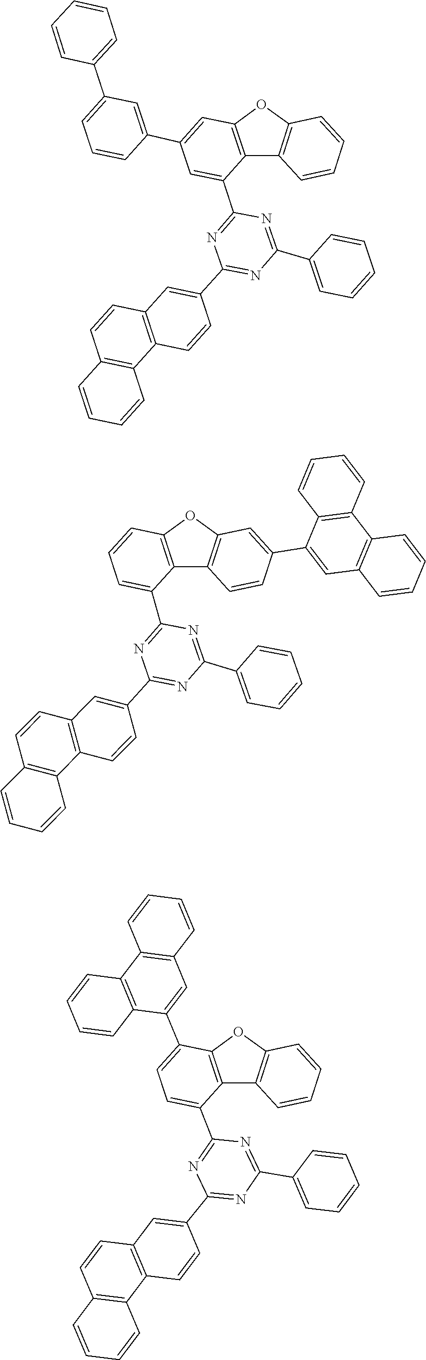 [SMILES: c1ccc(-c2cccc(-c3cc(-c4nc(-c5ccccc5)nc(-c5ccc6c(ccc7ccccc76)c5)n4)c4c(c3)oc3ccccc34)c2)cc1 ; c1ccc(-c2nc(-c3ccc4c(ccc5ccccc54)c3)nc(-c3cccc4oc5cc(-c6cc7ccccc7c7ccccc67)ccc5c34)n2)cc1 ; c1ccc(-c2nc(-c3ccc4c(ccc5ccccc54)c3)nc(-c3ccc(-c4cc5ccccc5c5ccccc45)c4oc5ccccc5c34)n2)cc1]

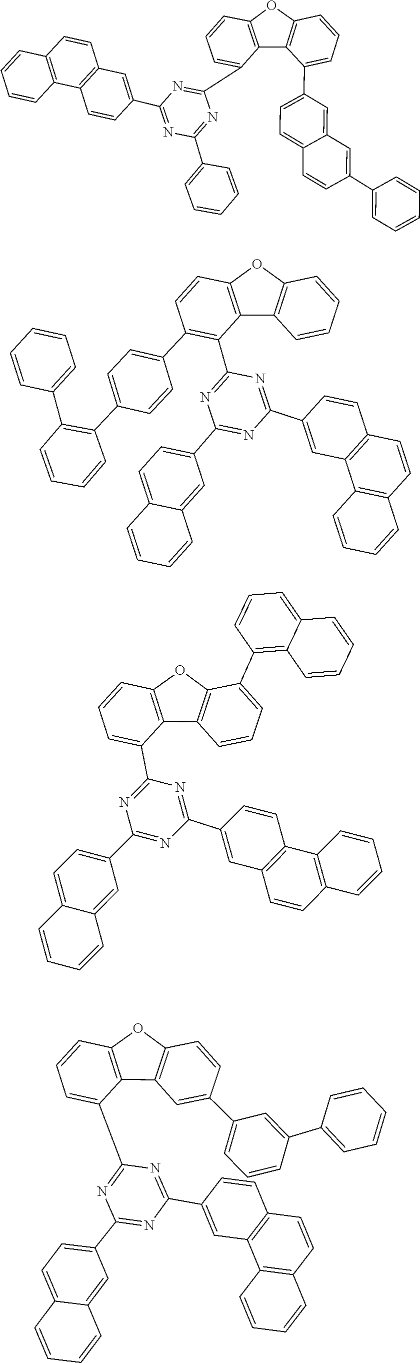 [SMILES: c1ccc(-c2ccc3ccc(-c4cccc5oc6cccc(-c7nc(-c8ccccc8)nc(-c8ccc9c(ccc%10ccccc%109)c8)n7)c6c45)cc3c2)cc1 ; c1ccc(-c2ccccc2-c2ccc(-c3ccc4oc5ccccc5c4c3-c3nc(-c4ccc5ccccc5c4)nc(-c4ccc5ccc6ccccc6c5c4)n3)cc2)cc1 ; c1ccc2cc(-c3nc(-c4ccc5c(ccc6ccccc65)c4)nc(-c4cccc5oc6c(-c7cccc8ccccc78)cccc6c45)n3)ccc2c1 ; c1ccc(-c2cccc(-c3ccc4oc5cccc(-c6nc(-c7ccc8ccccc8c7)nc(-c7ccc8ccc9ccccc9c8c7)n6)c5c4c3)c2)cc1]

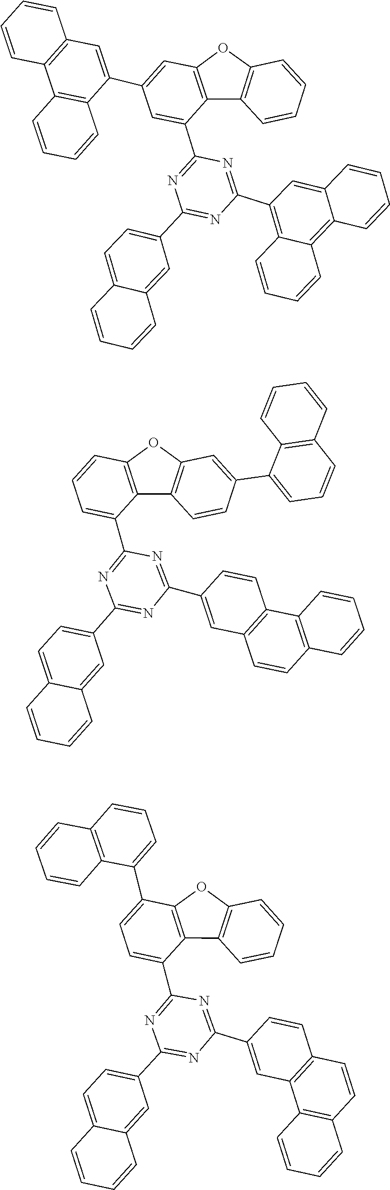 [SMILES: c1ccc2cc(-c3nc(-c4cc5ccccc5c5ccccc45)nc(-c4cc(-c5cc6ccccc6c6ccccc56)cc5oc6ccccc6c45)n3)ccc2c1 ; c1ccc2cc(-c3nc(-c4ccc5c(ccc6ccccc65)c4)nc(-c4cccc5oc6cc(-c7cccc8ccccc78)ccc6c45)n3)ccc2c1 ; c1ccc2cc(-c3nc(-c4ccc5ccc6ccccc6c5c4)nc(-c4ccc(-c5cccc6ccccc56)c5oc6ccccc6c45)n3)ccc2c1]

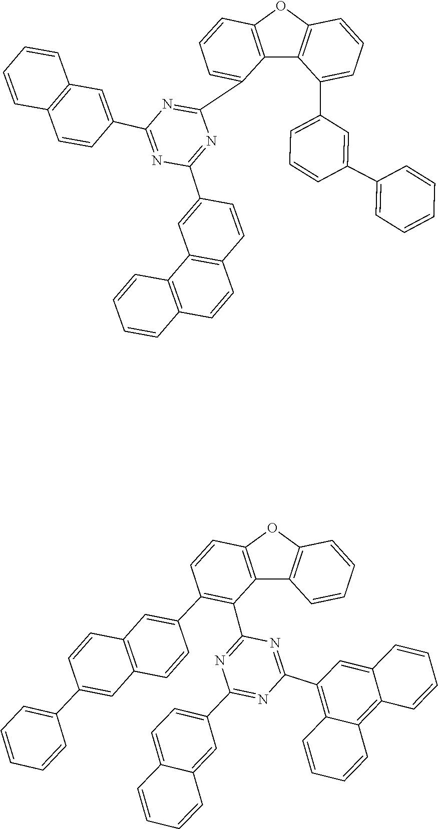 [SMILES: c1ccc(-c2cccc(-c3cccc4oc5cccc(-c6nc(-c7ccc8ccccc8c7)nc(-c7ccc8ccc9ccccc9c8c7)n6)c5c34)c2)cc1 ; c1ccc(-c2ccc3cc(-c4ccc5oc6ccccc6c5c4-c4nc(-c5ccc6ccccc6c5)nc(-c5cc6ccccc6c6ccccc56)n4)ccc3c2)cc1]

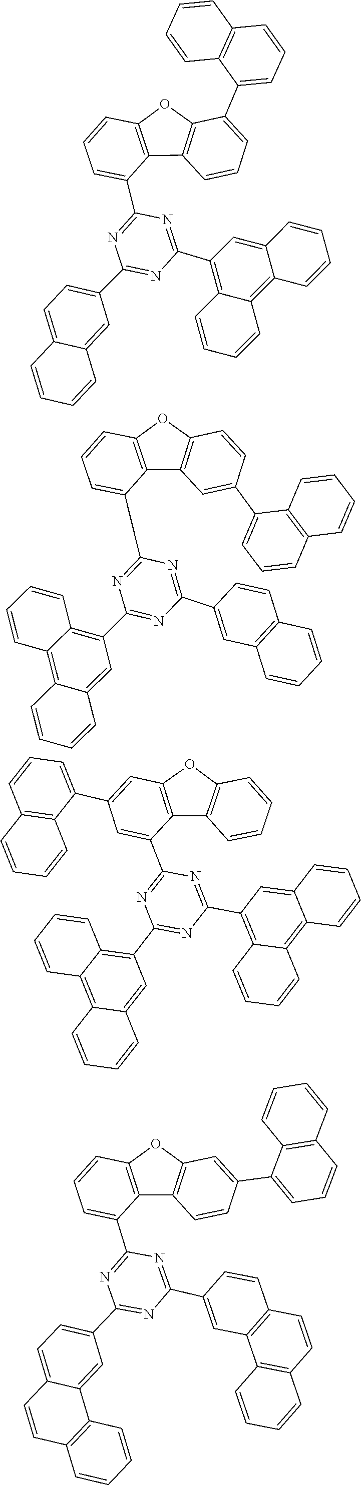

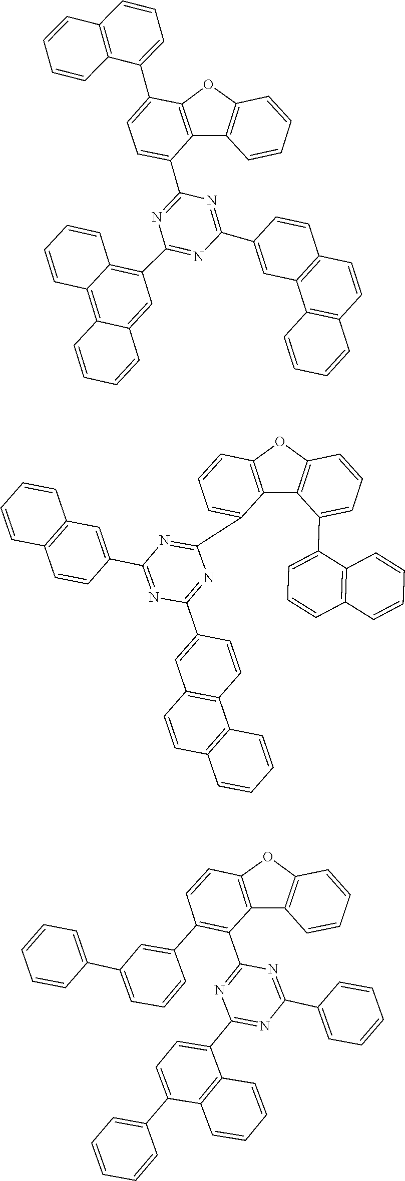 [SMILES: c1ccc2c(-c3ccc(-c4nc(-c5ccc6ccc7ccccc7c6c5)nc(-c5cc6ccccc6c6ccccc56)n4)c4c3oc3ccccc34)cccc2c1 ; c1ccc2cc(-c3nc(-c4ccc5c(ccc6ccccc65)c4)nc(-c4cccc5oc6cccc(-c7cccc8ccccc78)c6c45)n3)ccc2c1 ; c1ccc(-c2cccc(-c3ccc4oc5ccccc5c4c3-c3nc(-c4ccccc4)nc(-c4ccc(-c5ccccc5)c5ccccc45)n3)c2)cc1]

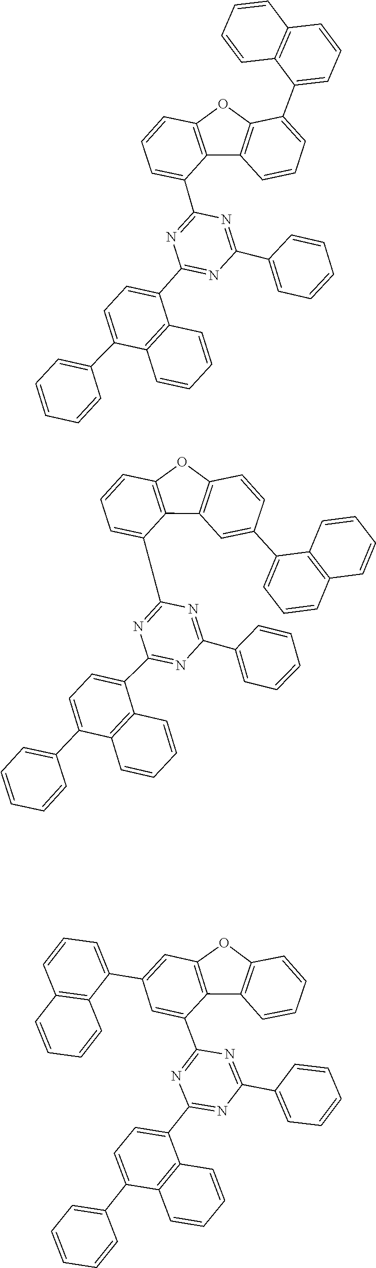 [SMILES: c1ccc(-c2nc(-c3ccc(-c4ccccc4)c4ccccc34)nc(-c3cccc4oc5c(-c6cccc7ccccc67)cccc5c34)n2)cc1 ; c1ccc(-c2nc(-c3ccc(-c4ccccc4)c4ccccc34)nc(-c3cccc4oc5ccc(-c6cccc7ccccc67)cc5c34)n2)cc1 ; c1ccc(-c2nc(-c3ccc(-c4ccccc4)c4ccccc34)nc(-c3cc(-c4cccc5ccccc45)cc4oc5ccccc5c34)n2)cc1]

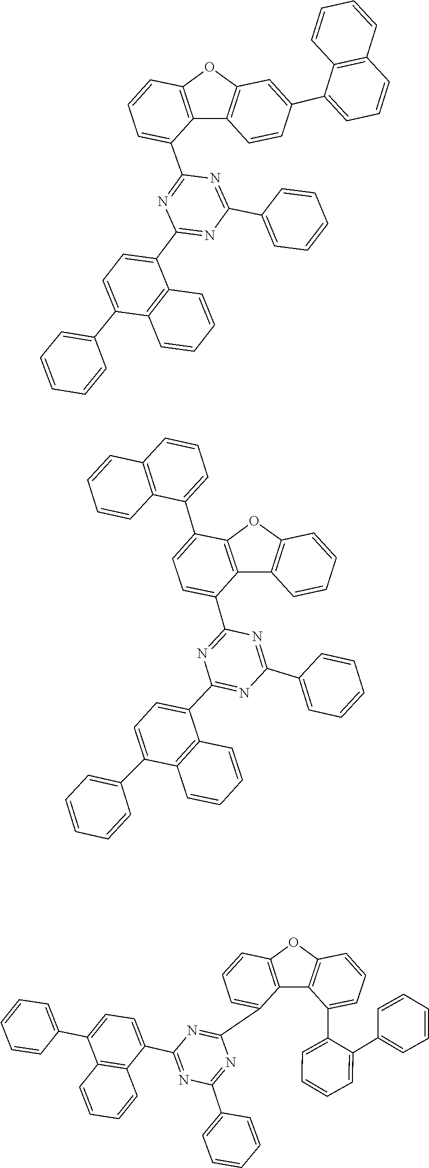 [SMILES: c1ccc(-c2nc(-c3ccc(-c4ccccc4)c4ccccc34)nc(-c3cccc4oc5cc(-c6cccc7ccccc67)ccc5c34)n2)cc1 ; c1ccc(-c2nc(-c3ccc(-c4ccccc4)c4ccccc34)nc(-c3ccc(-c4cccc5ccccc45)c4oc5ccccc5c34)n2)cc1 ; c1ccc(-c2nc(-c3ccc(-c4ccccc4)c4ccccc34)nc(-c3cccc4oc5cccc(-c6ccccc6-c6ccccc6)c5c34)n2)cc1]

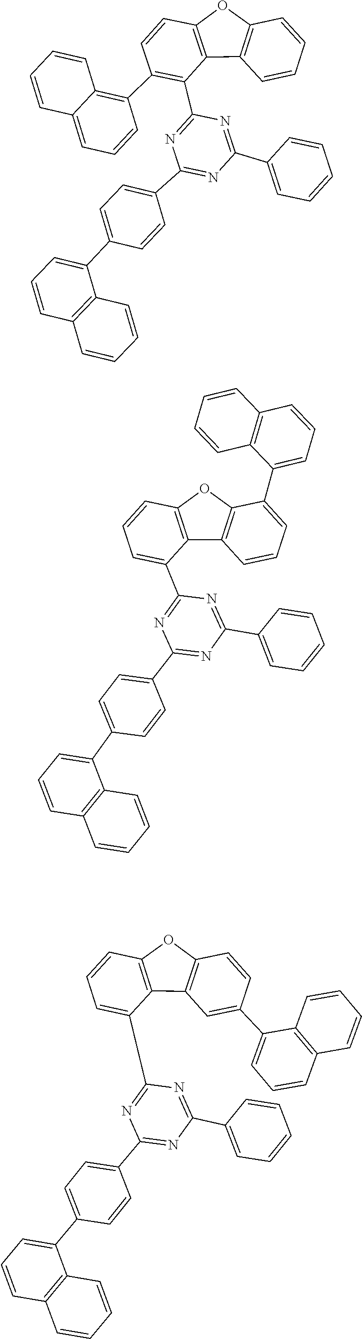 [SMILES: c1ccc(-c2nc(-c3ccc(-c4cccc5ccccc45)cc3)nc(-c3c(-c4cccc5ccccc45)ccc4oc5ccccc5c34)n2)cc1 ; c1ccc(-c2nc(-c3ccc(-c4cccc5ccccc45)cc3)nc(-c3cccc4oc5c(-c6cccc7ccccc67)cccc5c34)n2)cc1 ; c1ccc(-c2nc(-c3ccc(-c4cccc5ccccc45)cc3)nc(-c3cccc4oc5ccc(-c6cccc7ccccc67)cc5c34)n2)cc1]

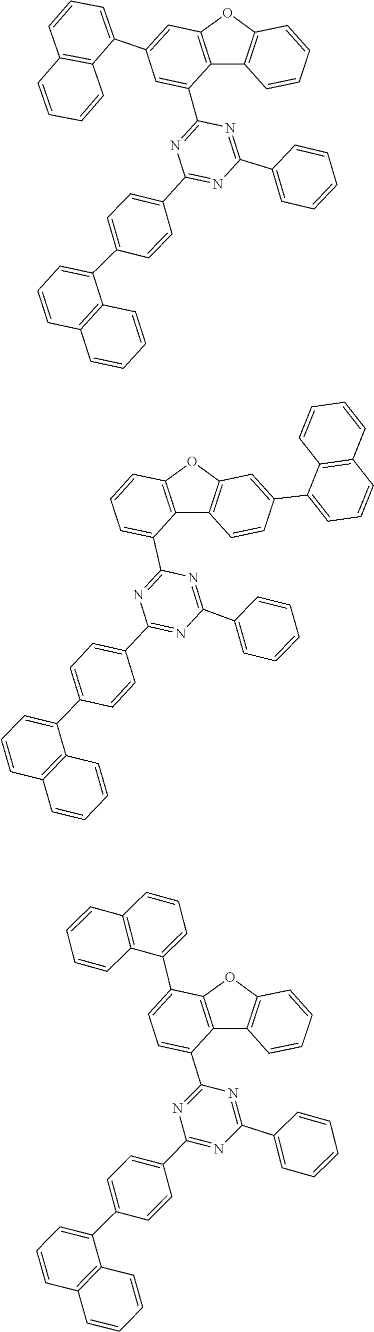 [SMILES: c1ccc(-c2nc(-c3ccc(-c4cccc5ccccc45)cc3)nc(-c3cc(-c4cccc5ccccc45)cc4oc5ccccc5c34)n2)cc1 ; c1ccc(-c2nc(-c3ccc(-c4cccc5ccccc45)cc3)nc(-c3cccc4oc5cc(-c6cccc7ccccc67)ccc5c34)n2)cc1 ; c1ccc(-c2nc(-c3ccc(-c4cccc5ccccc45)cc3)nc(-c3ccc(-c4cccc5ccccc45)c4oc5ccccc5c34)n2)cc1]

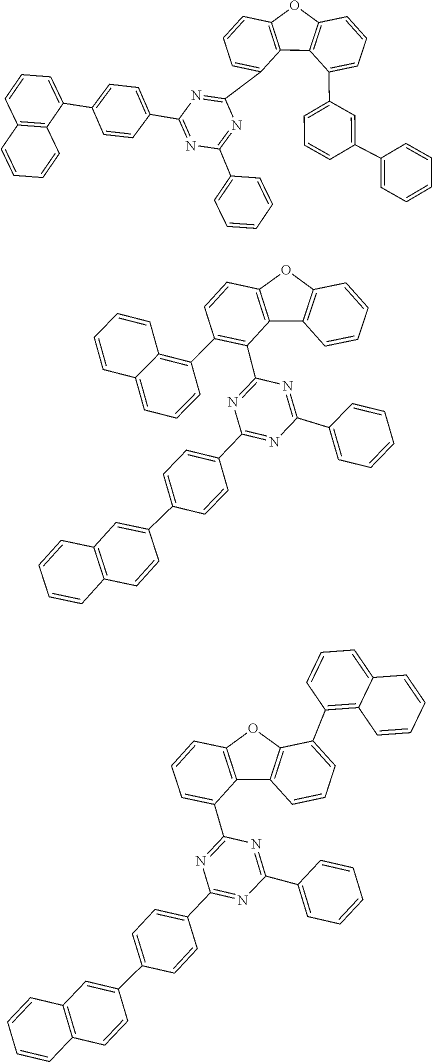 [SMILES: c1ccc(-c2cccc(-c3cccc4oc5cccc(-c6nc(-c7ccccc7)nc(-c7ccc(-c8cccc9ccccc89)cc7)n6)c5c34)c2)cc1 ; c1ccc(-c2nc(-c3ccc(-c4ccc5ccccc5c4)cc3)nc(-c3c(-c4cccc5ccccc45)ccc4oc5ccccc5c34)n2)cc1 ; c1ccc(-c2nc(-c3ccc(-c4ccc5ccccc5c4)cc3)nc(-c3cccc4oc5c(-c6cccc7ccccc67)cccc5c34)n2)cc1]

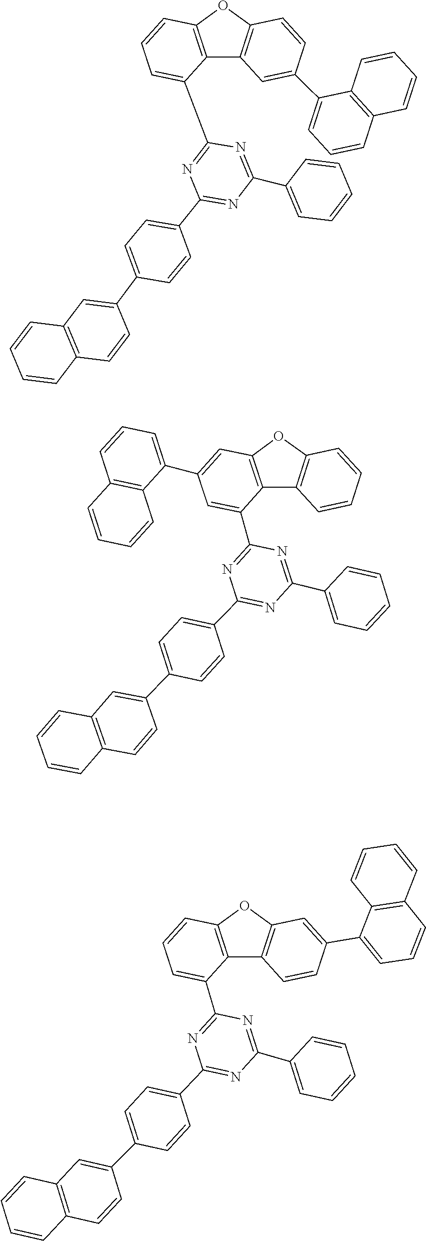 [SMILES: c1ccc(-c2nc(-c3ccc(-c4ccc5ccccc5c4)cc3)nc(-c3cccc4oc5ccc(-c6cccc7ccccc67)cc5c34)n2)cc1 ; c1ccc(-c2nc(-c3ccc(-c4ccc5ccccc5c4)cc3)nc(-c3cc(-c4cccc5ccccc45)cc4oc5ccccc5c34)n2)cc1 ; c1ccc(-c2nc(-c3ccc(-c4ccc5ccccc5c4)cc3)nc(-c3cccc4oc5cc(-c6cccc7ccccc67)ccc5c34)n2)cc1]

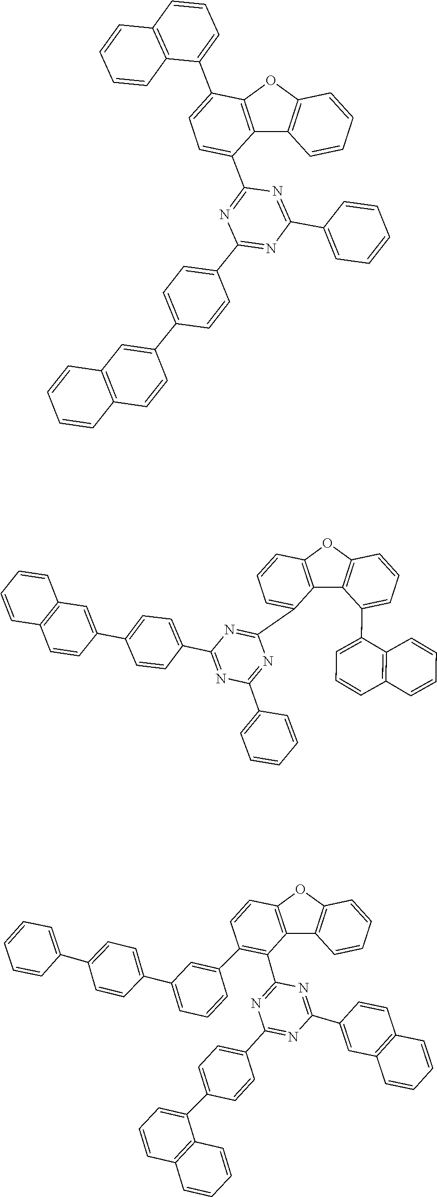 [SMILES: c1ccc(-c2nc(-c3ccc(-c4ccc5ccccc5c4)cc3)nc(-c3ccc(-c4cccc5ccccc45)c4oc5ccccc5c34)n2)cc1 ; c1ccc(-c2nc(-c3ccc(-c4ccc5ccccc5c4)cc3)nc(-c3cccc4oc5cccc(-c6cccc7ccccc67)c5c34)n2)cc1 ; c1ccc(-c2ccc(-c3cccc(-c4ccc5oc6ccccc6c5c4-c4nc(-c5ccc(-c6cccc7ccccc67)cc5)nc(-c5ccc6ccccc6c5)n4)c3)cc2)cc1]

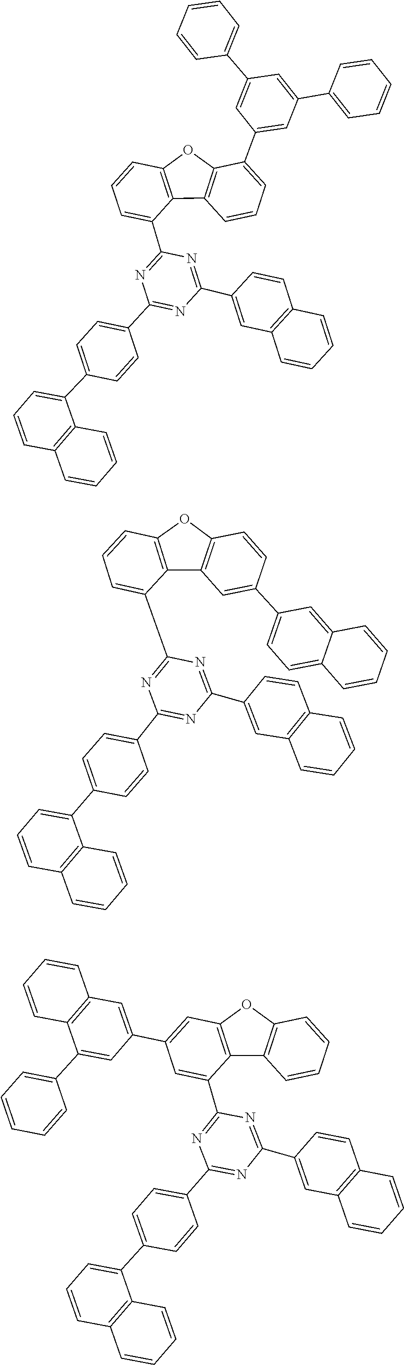 [SMILES: c1ccc(-c2cc(-c3ccccc3)cc(-c3cccc4c3oc3cccc(-c5nc(-c6ccc(-c7cccc8ccccc78)cc6)nc(-c6ccc7ccccc7c6)n5)c34)c2)cc1 ; c1ccc2cc(-c3ccc4oc5cccc(-c6nc(-c7ccc(-c8cccc9ccccc89)cc7)nc(-c7ccc8ccccc8c7)n6)c5c4c3)ccc2c1 ; c1ccc(-c2cc(-c3cc(-c4nc(-c5ccc(-c6cccc7ccccc67)cc5)nc(-c5ccc6ccccc6c5)n4)c4c(c3)oc3ccccc34)cc3ccccc23)cc1]

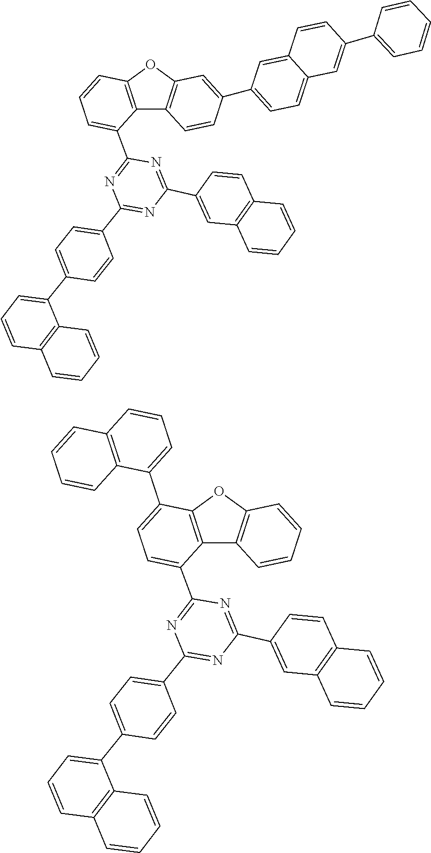 [SMILES: c1ccc(-c2ccc3cc(-c4ccc5c(c4)oc4cccc(-c6nc(-c7ccc(-c8cccc9ccccc89)cc7)nc(-c7ccc8ccccc8c7)n6)c45)ccc3c2)cc1 ; c1ccc2cc(-c3nc(-c4ccc(-c5cccc6ccccc56)cc4)nc(-c4ccc(-c5cccc6ccccc56)c5oc6ccccc6c45)n3)ccc2c1]

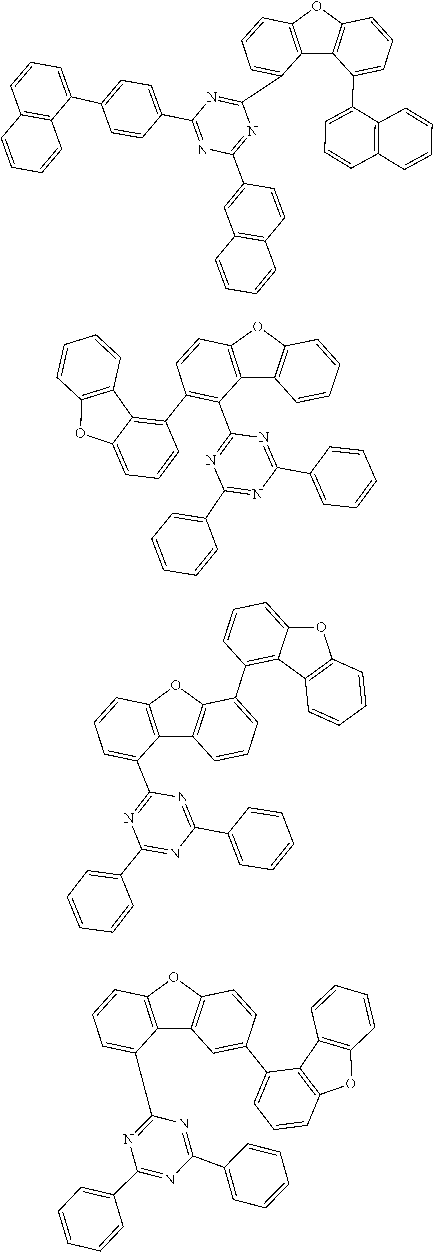

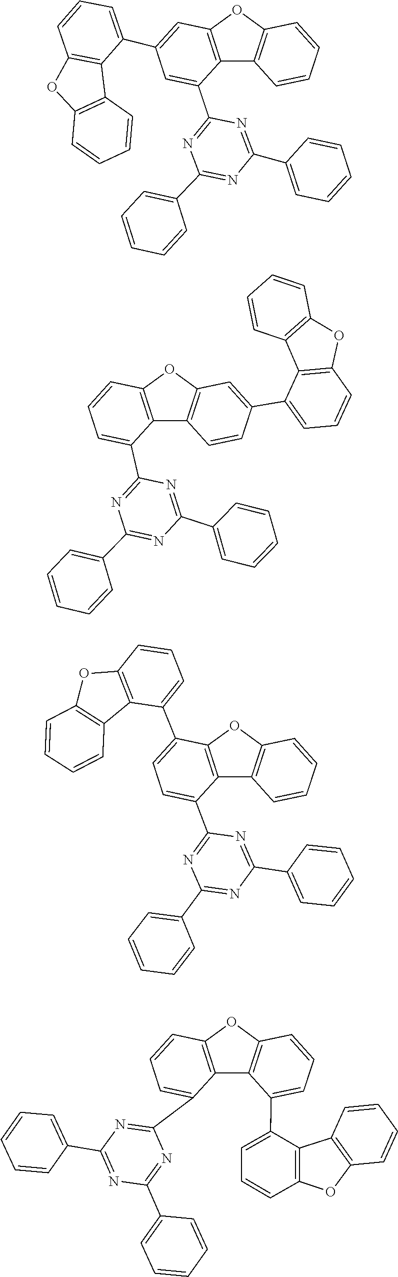 [SMILES: c1ccc(-c2nc(-c3ccccc3)nc(-c3cc(-c4cccc5oc6ccccc6c45)cc4oc5ccccc5c34)n2)cc1 ; c1ccc(-c2nc(-c3ccccc3)nc(-c3cccc4oc5cc(-c6cccc7oc8ccccc8c67)ccc5c34)n2)cc1 ; c1ccc(-c2nc(-c3ccccc3)nc(-c3ccc(-c4cccc5oc6ccccc6c45)c4oc5ccccc5c34)n2)cc1 ; c1ccc(-c2nc(-c3ccccc3)nc(-c3cccc4oc5cccc(-c6cccc7oc8ccccc8c67)c5c34)n2)cc1]

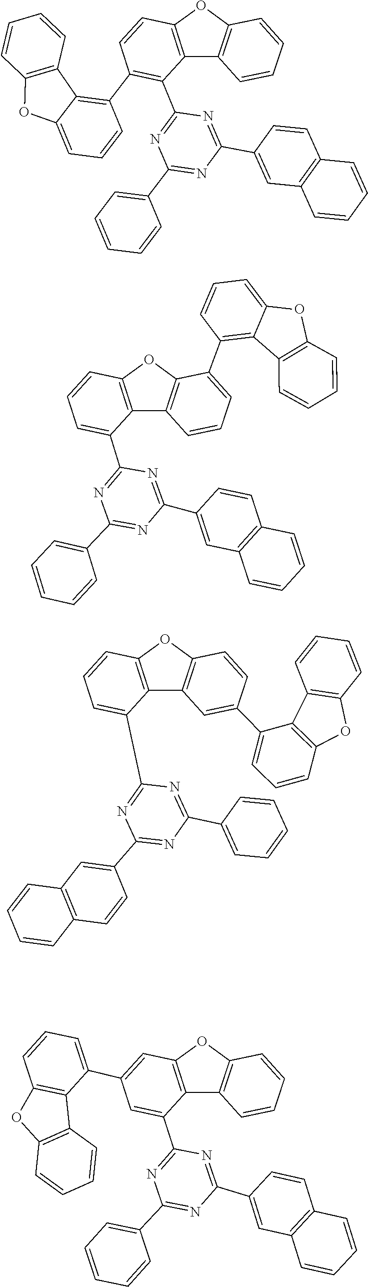 [SMILES: c1ccc(-c2nc(-c3ccc4ccccc4c3)nc(-c3c(-c4cccc5oc6ccccc6c45)ccc4oc5ccccc5c34)n2)cc1 ; c1ccc(-c2nc(-c3ccc4ccccc4c3)nc(-c3cccc4oc5c(-c6cccc7oc8ccccc8c67)cccc5c34)n2)cc1 ; c1ccc(-c2nc(-c3ccc4ccccc4c3)nc(-c3cccc4oc5ccc(-c6cccc7oc8ccccc8c67)cc5c34)n2)cc1 ; c1ccc(-c2nc(-c3ccc4ccccc4c3)nc(-c3cc(-c4cccc5oc6ccccc6c45)cc4oc5ccccc5c34)n2)cc1]

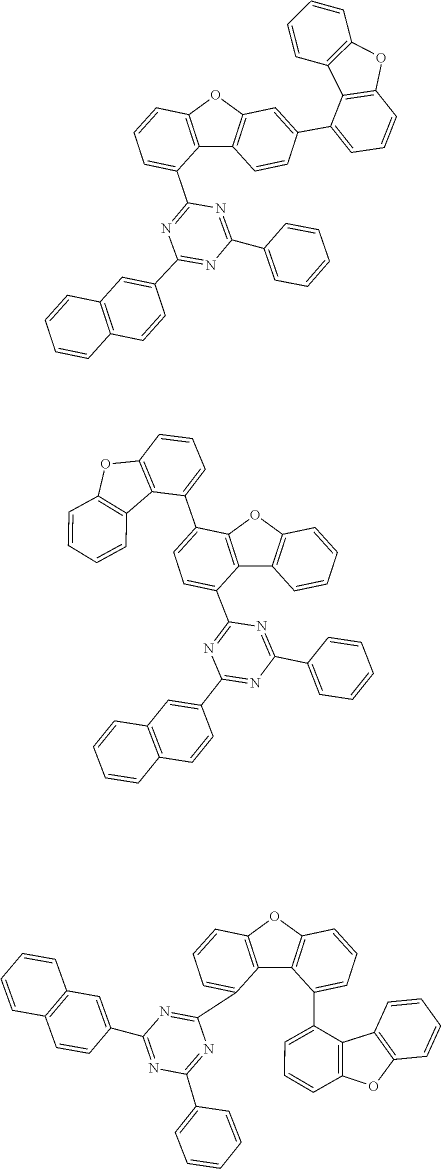 [SMILES: c1ccc(-c2nc(-c3ccc4ccccc4c3)nc(-c3cccc4oc5cc(-c6cccc7oc8ccccc8c67)ccc5c34)n2)cc1 ; c1ccc(-c2nc(-c3ccc4ccccc4c3)nc(-c3ccc(-c4cccc5oc6ccccc6c45)c4oc5ccccc5c34)n2)cc1 ; c1ccc(-c2nc(-c3ccc4ccccc4c3)nc(-c3cccc4oc5cccc(-c6cccc7oc8ccccc8c67)c5c34)n2)cc1]

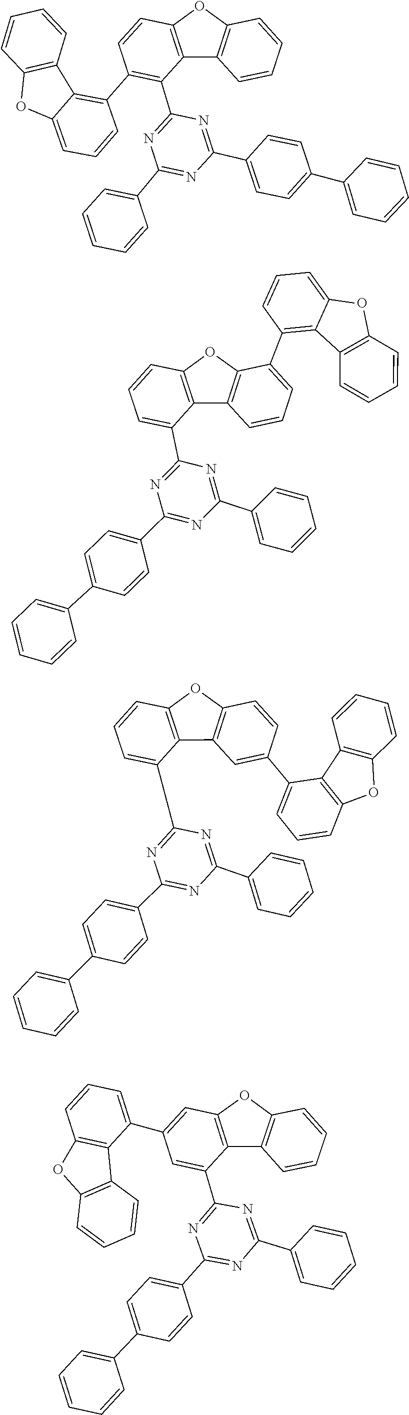 [SMILES: c1ccc(-c2ccc(-c3nc(-c4ccccc4)nc(-c4c(-c5cccc6oc7ccccc7c56)ccc5oc6ccccc6c45)n3)cc2)cc1 ; c1ccc(-c2ccc(-c3nc(-c4ccccc4)nc(-c4cccc5oc6c(-c7cccc8oc9ccccc9c78)cccc6c45)n3)cc2)cc1 ; c1ccc(-c2ccc(-c3nc(-c4ccccc4)nc(-c4cccc5oc6ccc(-c7cccc8oc9ccccc9c78)cc6c45)n3)cc2)cc1 ; c1ccc(-c2ccc(-c3nc(-c4ccccc4)nc(-c4cc(-c5cccc6oc7ccccc7c56)cc5oc6ccccc6c45)n3)cc2)cc1]

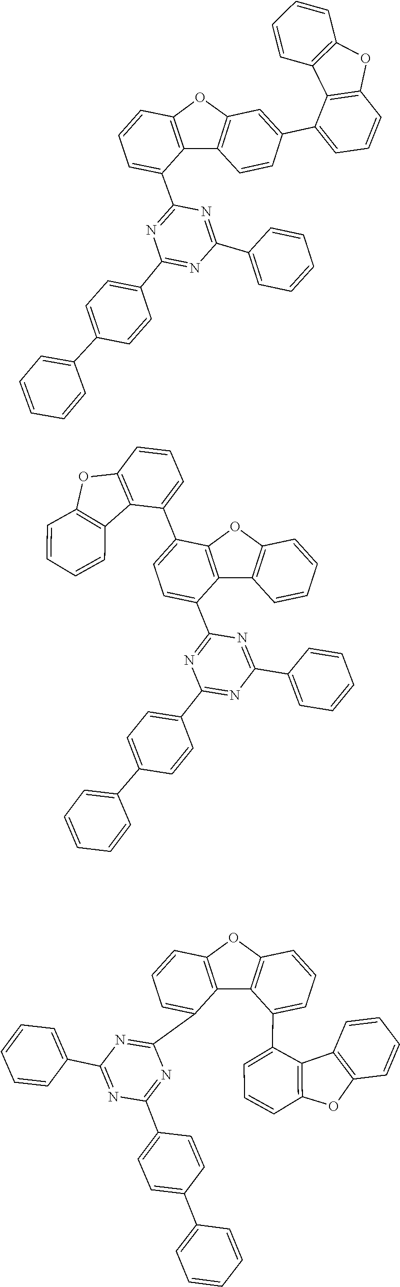 [SMILES: c1ccc(-c2ccc(-c3nc(-c4ccccc4)nc(-c4cccc5oc6cc(-c7cccc8oc9ccccc9c78)ccc6c45)n3)cc2)cc1 ; c1ccc(-c2ccc(-c3nc(-c4ccccc4)nc(-c4ccc(-c5cccc6oc7ccccc7c56)c5oc6ccccc6c45)n3)cc2)cc1 ; c1ccc(-c2ccc(-c3nc(-c4ccccc4)nc(-c4cccc5oc6cccc(-c7cccc8oc9ccccc9c78)c6c45)n3)cc2)cc1]

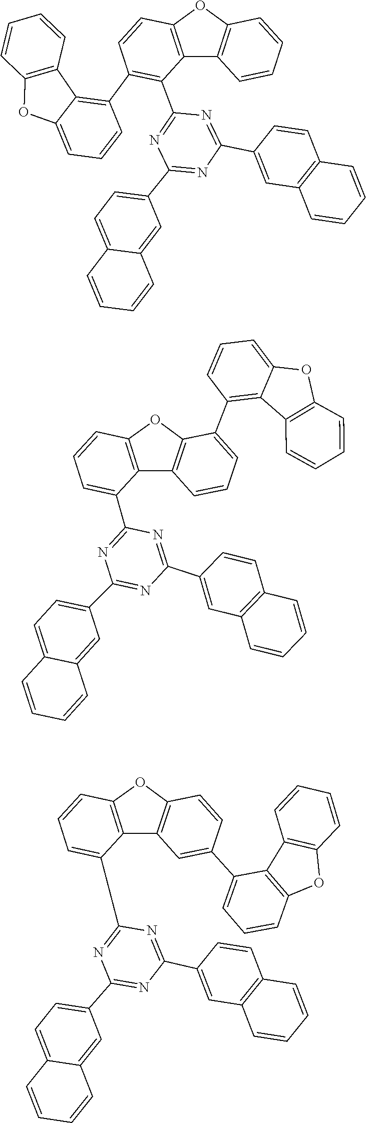 [SMILES: c1ccc2cc(-c3nc(-c4ccc5ccccc5c4)nc(-c4c(-c5cccc6oc7ccccc7c56)ccc5oc6ccccc6c45)n3)ccc2c1 ; c1ccc2cc(-c3nc(-c4ccc5ccccc5c4)nc(-c4cccc5oc6c(-c7cccc8oc9ccccc9c78)cccc6c45)n3)ccc2c1 ; c1ccc2cc(-c3nc(-c4ccc5ccccc5c4)nc(-c4cccc5oc6ccc(-c7cccc8oc9ccccc9c78)cc6c45)n3)ccc2c1]

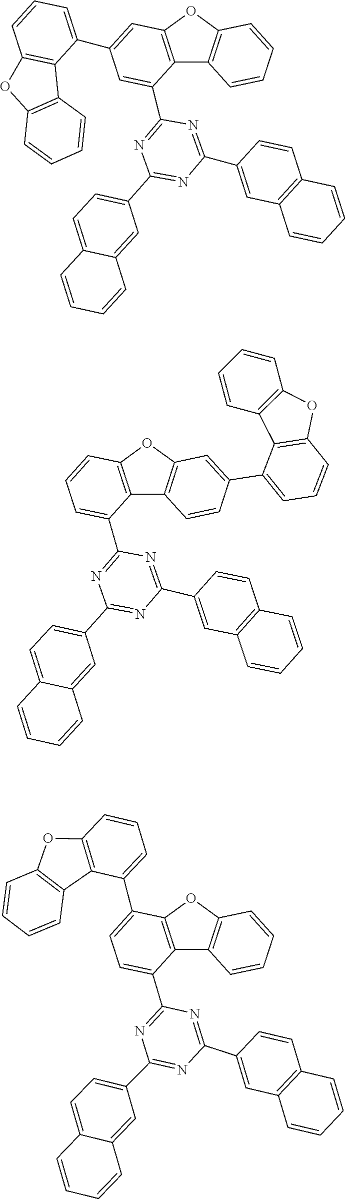 [SMILES: c1ccc2cc(-c3nc(-c4ccc5ccccc5c4)nc(-c4cc(-c5cccc6oc7ccccc7c56)cc5oc6ccccc6c45)n3)ccc2c1 ; c1ccc2cc(-c3nc(-c4ccc5ccccc5c4)nc(-c4cccc5oc6cc(-c7cccc8oc9ccccc9c78)ccc6c45)n3)ccc2c1 ; c1ccc2cc(-c3nc(-c4ccc5ccccc5c4)nc(-c4ccc(-c5cccc6oc7ccccc7c56)c5oc6ccccc6c45)n3)ccc2c1]

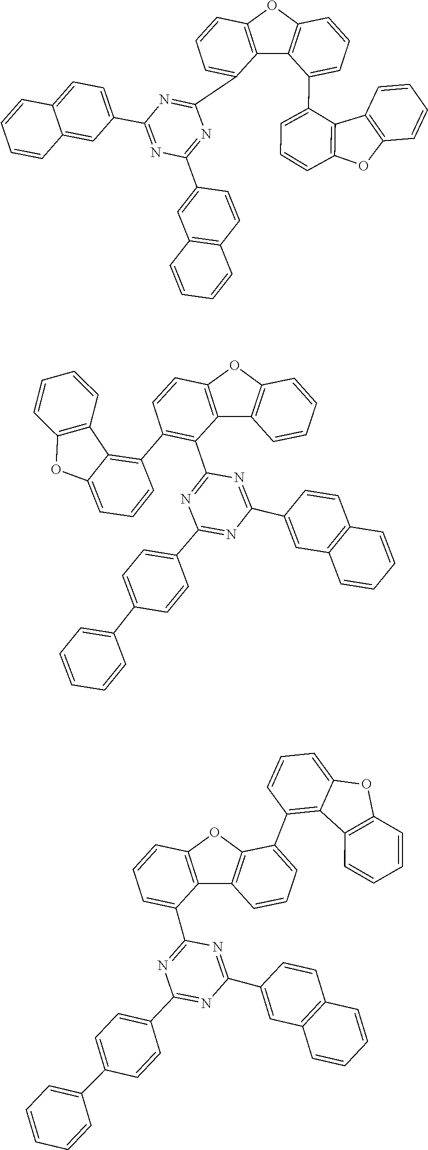 [SMILES: c1ccc2cc(-c3nc(-c4ccc5ccccc5c4)nc(-c4cccc5oc6cccc(-c7cccc8oc9ccccc9c78)c6c45)n3)ccc2c1 ; c1ccc(-c2ccc(-c3nc(-c4ccc5ccccc5c4)nc(-c4c(-c5cccc6oc7ccccc7c56)ccc5oc6ccccc6c45)n3)cc2)cc1 ; c1ccc(-c2ccc(-c3nc(-c4ccc5ccccc5c4)nc(-c4cccc5oc6c(-c7cccc8oc9ccccc9c78)cccc6c45)n3)cc2)cc1]

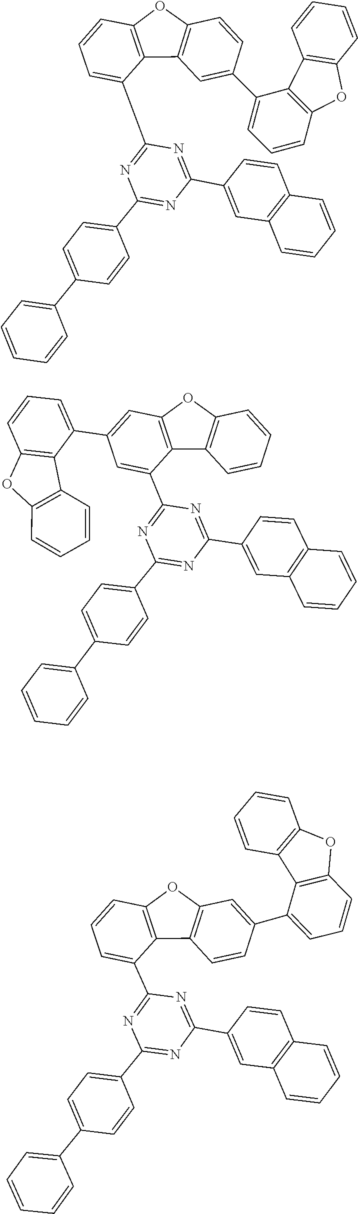

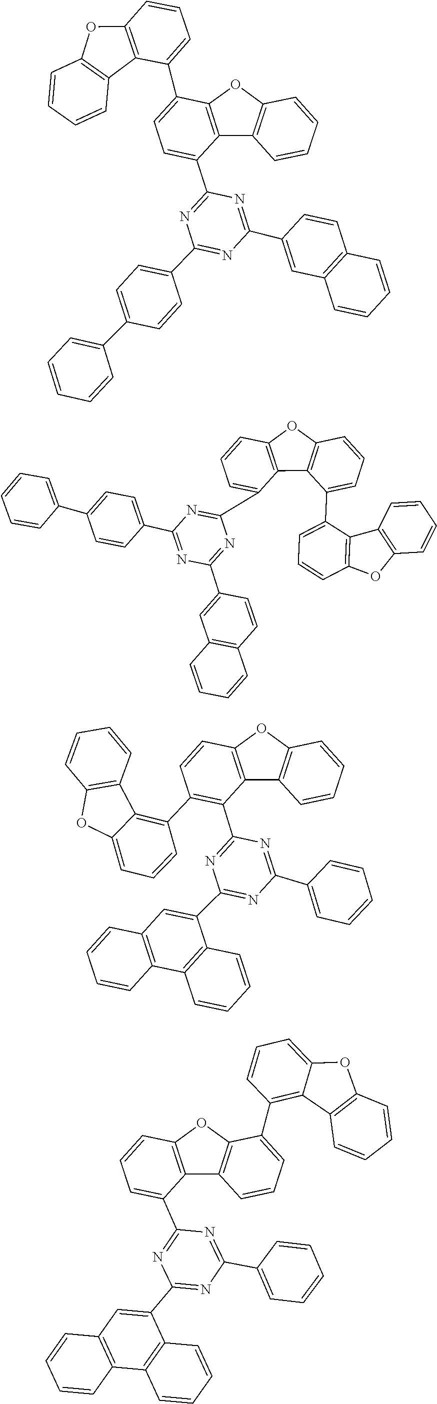

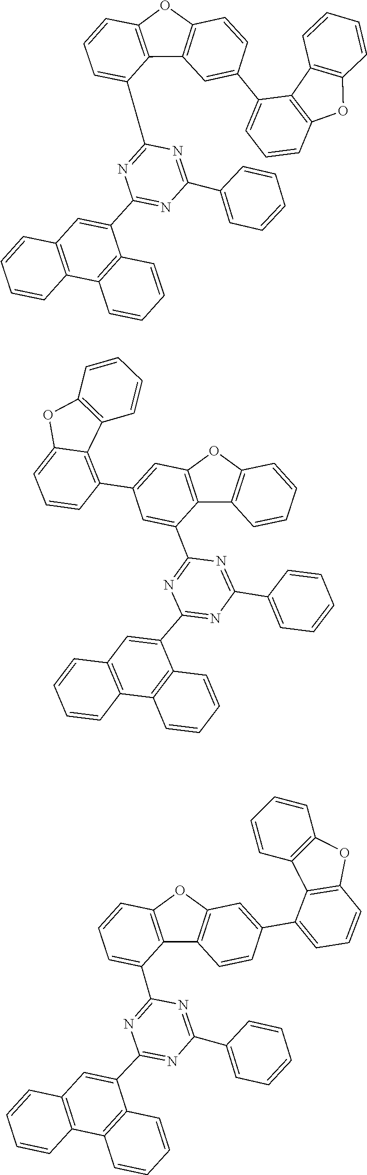

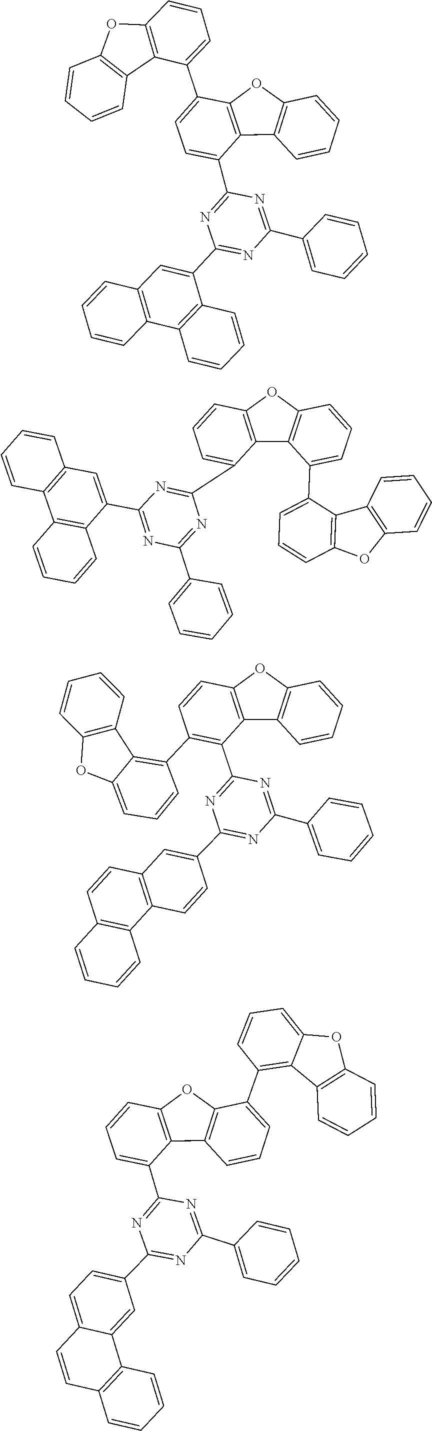

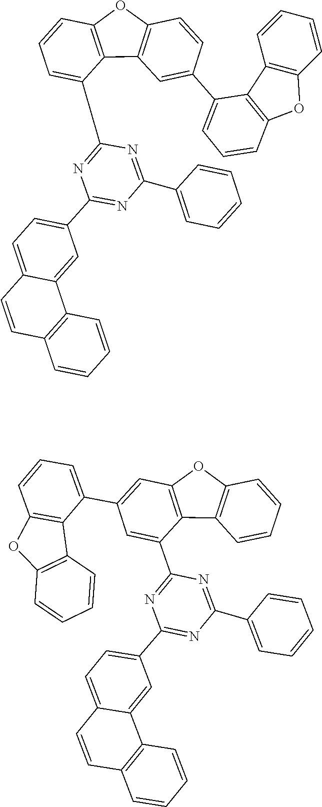 [SMILES: c1ccc(-c2nc(-c3ccc4ccc5ccccc5c4c3)nc(-c3cccc4oc5ccc(-c6cccc7oc8ccccc8c67)cc5c34)n2)cc1 ; c1ccc(-c2nc(-c3ccc4ccc5ccccc5c4c3)nc(-c3cc(-c4cccc5oc6ccccc6c45)cc4oc5ccccc5c34)n2)cc1]

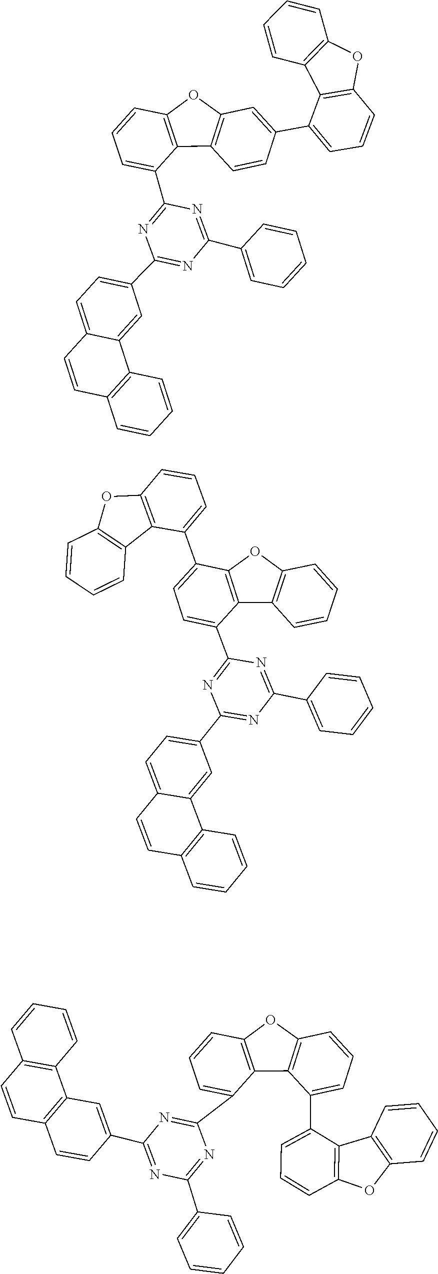

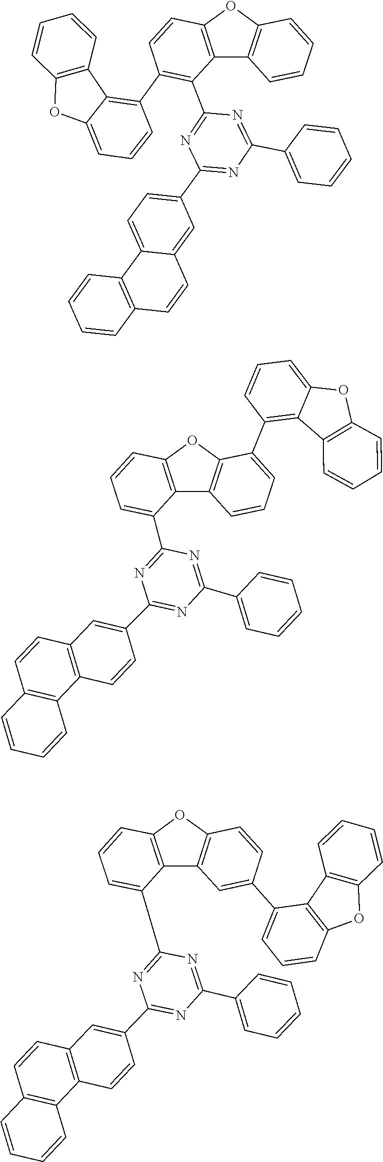 [SMILES: c1ccc(-c2nc(-c3ccc4c(ccc5ccccc54)c3)nc(-c3c(-c4cccc5oc6ccccc6c45)ccc4oc5ccccc5c34)n2)cc1 ; c1ccc(-c2nc(-c3ccc4c(ccc5ccccc54)c3)nc(-c3cccc4oc5c(-c6cccc7oc8ccccc8c67)cccc5c34)n2)cc1 ; c1ccc(-c2nc(-c3ccc4c(ccc5ccccc54)c3)nc(-c3cccc4oc5ccc(-c6cccc7oc8ccccc8c67)cc5c34)n2)cc1]

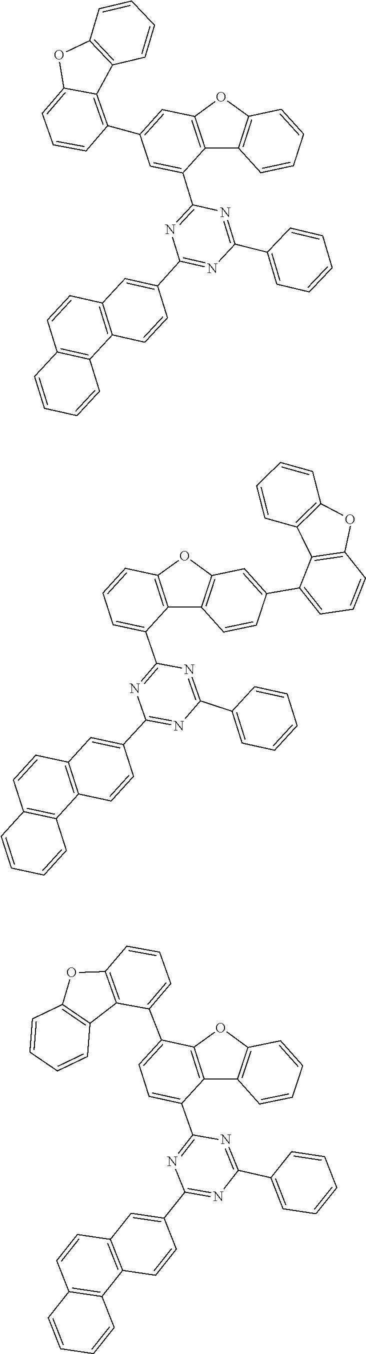 [SMILES: c1ccc(-c2nc(-c3ccc4c(ccc5ccccc54)c3)nc(-c3cc(-c4cccc5oc6ccccc6c45)cc4oc5ccccc5c34)n2)cc1 ; c1ccc(-c2nc(-c3ccc4c(ccc5ccccc54)c3)nc(-c3cccc4oc5cc(-c6cccc7oc8ccccc8c67)ccc5c34)n2)cc1 ; c1ccc(-c2nc(-c3ccc4c(ccc5ccccc54)c3)nc(-c3ccc(-c4cccc5oc6ccccc6c45)c4oc5ccccc5c34)n2)cc1]

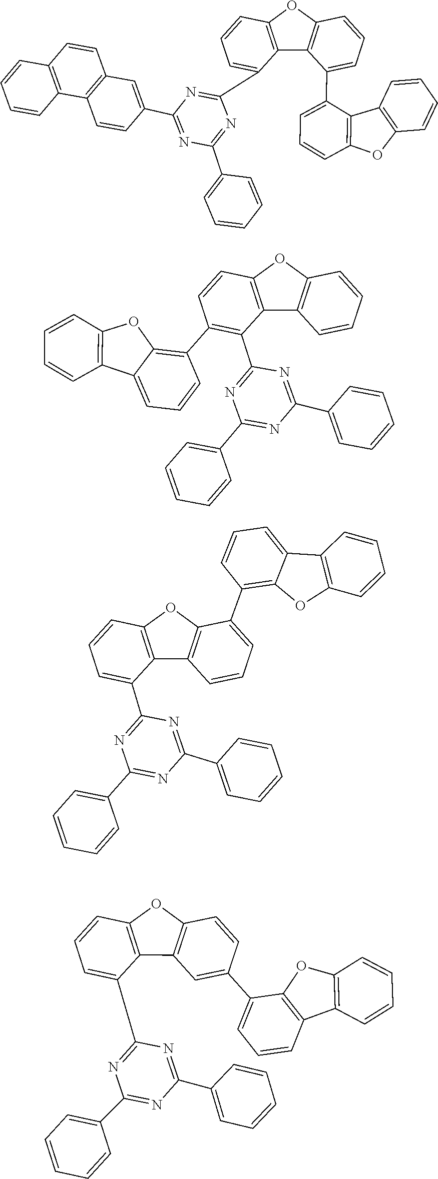

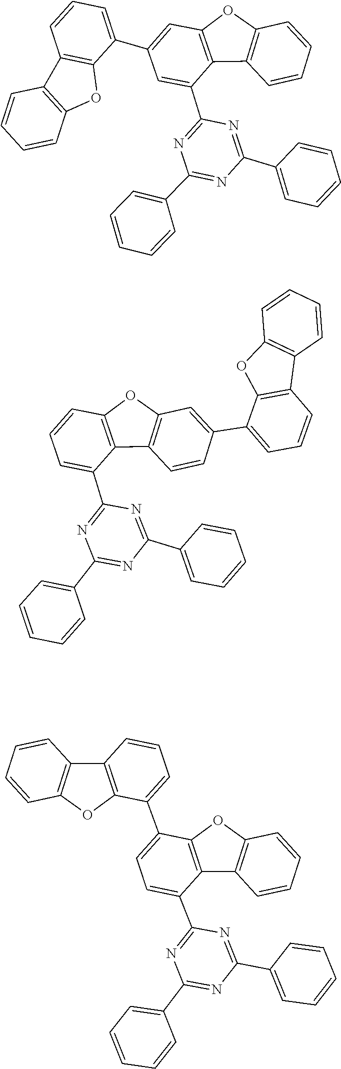 [SMILES: c1ccc(-c2nc(-c3ccccc3)nc(-c3cc(-c4cccc5c4oc4ccccc45)cc4oc5ccccc5c34)n2)cc1 ; c1ccc(-c2nc(-c3ccccc3)nc(-c3cccc4oc5cc(-c6cccc7c6oc6ccccc67)ccc5c34)n2)cc1 ; c1ccc(-c2nc(-c3ccccc3)nc(-c3ccc(-c4cccc5c4oc4ccccc45)c4oc5ccccc5c34)n2)cc1]

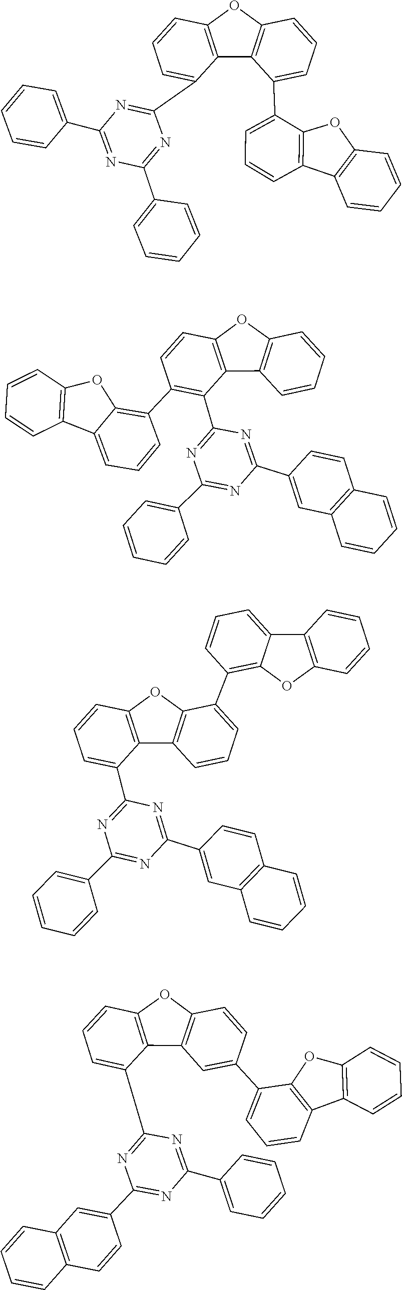 [SMILES: c1ccc(-c2nc(-c3ccccc3)nc(-c3cccc4oc5cccc(-c6cccc7c6oc6ccccc67)c5c34)n2)cc1 ; c1ccc(-c2nc(-c3ccc4ccccc4c3)nc(-c3c(-c4cccc5c4oc4ccccc45)ccc4oc5ccccc5c34)n2)cc1 ; c1ccc(-c2nc(-c3ccc4ccccc4c3)nc(-c3cccc4oc5c(-c6cccc7c6oc6ccccc67)cccc5c34)n2)cc1 ; c1ccc(-c2nc(-c3ccc4ccccc4c3)nc(-c3cccc4oc5ccc(-c6cccc7c6oc6ccccc67)cc5c34)n2)cc1]

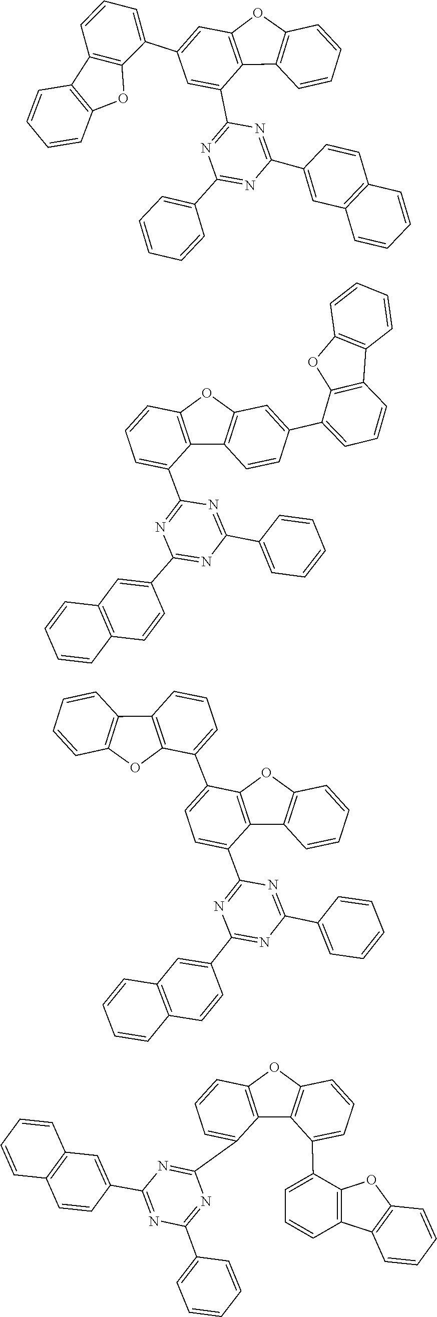

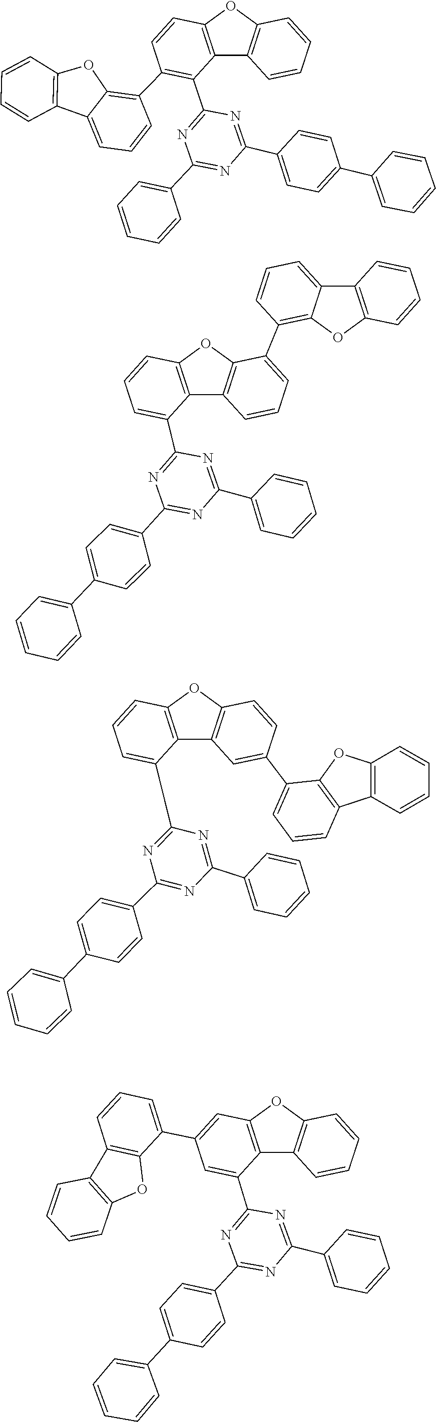 [SMILES: c1ccc(-c2ccc(-c3nc(-c4ccccc4)nc(-c4c(-c5cccc6c5oc5ccccc56)ccc5oc6ccccc6c45)n3)cc2)cc1 ; c1ccc(-c2ccc(-c3nc(-c4ccccc4)nc(-c4cccc5oc6c(-c7cccc8c7oc7ccccc78)cccc6c45)n3)cc2)cc1 ; c1ccc(-c2ccc(-c3nc(-c4ccccc4)nc(-c4cccc5oc6ccc(-c7cccc8c7oc7ccccc78)cc6c45)n3)cc2)cc1 ; c1ccc(-c2ccc(-c3nc(-c4ccccc4)nc(-c4cc(-c5cccc6c5oc5ccccc56)cc5oc6ccccc6c45)n3)cc2)cc1]

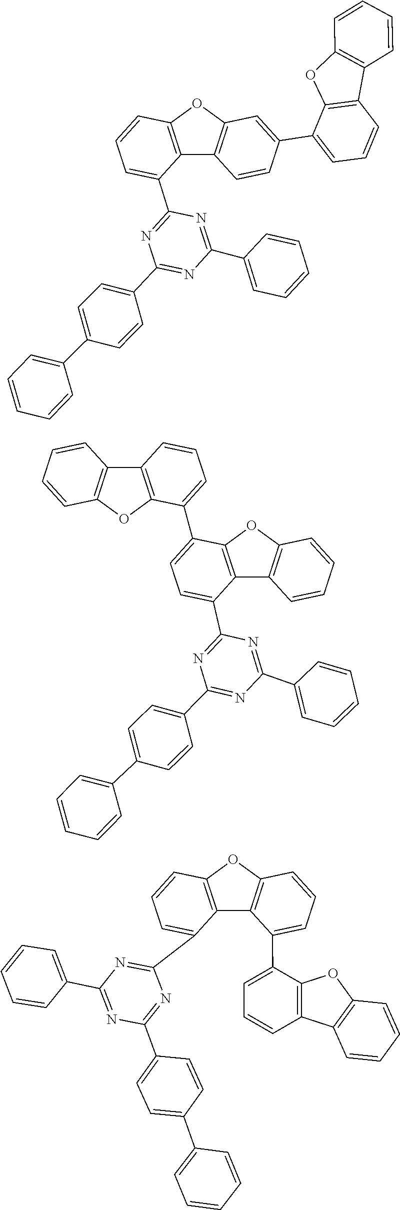

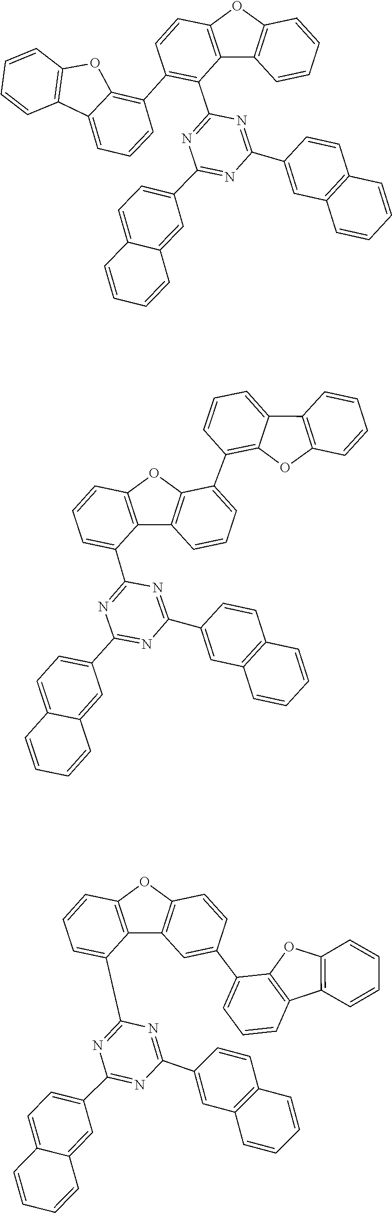 [SMILES: c1ccc2cc(-c3nc(-c4ccc5ccccc5c4)nc(-c4c(-c5cccc6c5oc5ccccc56)ccc5oc6ccccc6c45)n3)ccc2c1 ; c1ccc2cc(-c3nc(-c4ccc5ccccc5c4)nc(-c4cccc5oc6c(-c7cccc8c7oc7ccccc78)cccc6c45)n3)ccc2c1 ; c1ccc2cc(-c3nc(-c4ccc5ccccc5c4)nc(-c4cccc5oc6ccc(-c7cccc8c7oc7ccccc78)cc6c45)n3)ccc2c1]

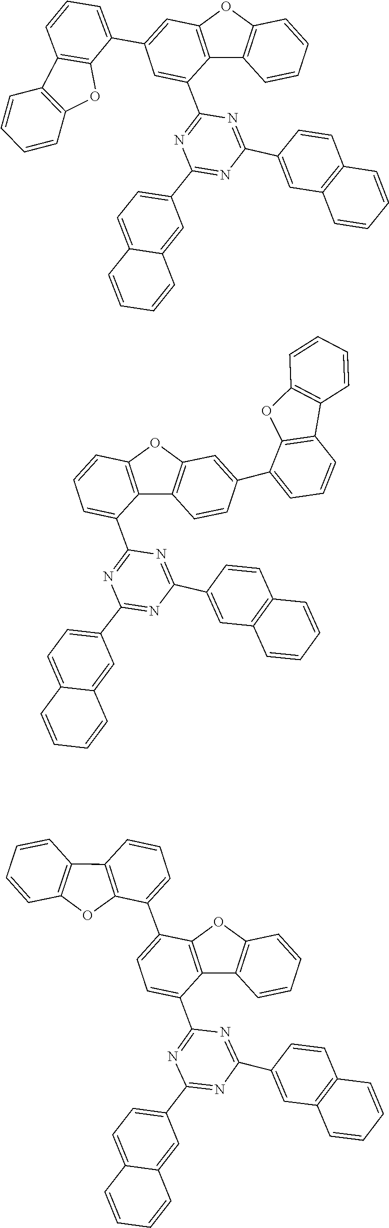 [SMILES: c1ccc2cc(-c3nc(-c4ccc5ccccc5c4)nc(-c4cc(-c5cccc6c5oc5ccccc56)cc5oc6ccccc6c45)n3)ccc2c1 ; c1ccc2cc(-c3nc(-c4ccc5ccccc5c4)nc(-c4cccc5oc6cc(-c7cccc8c7oc7ccccc78)ccc6c45)n3)ccc2c1 ; c1ccc2cc(-c3nc(-c4ccc5ccccc5c4)nc(-c4ccc(-c5cccc6c5oc5ccccc56)c5oc6ccccc6c45)n3)ccc2c1]

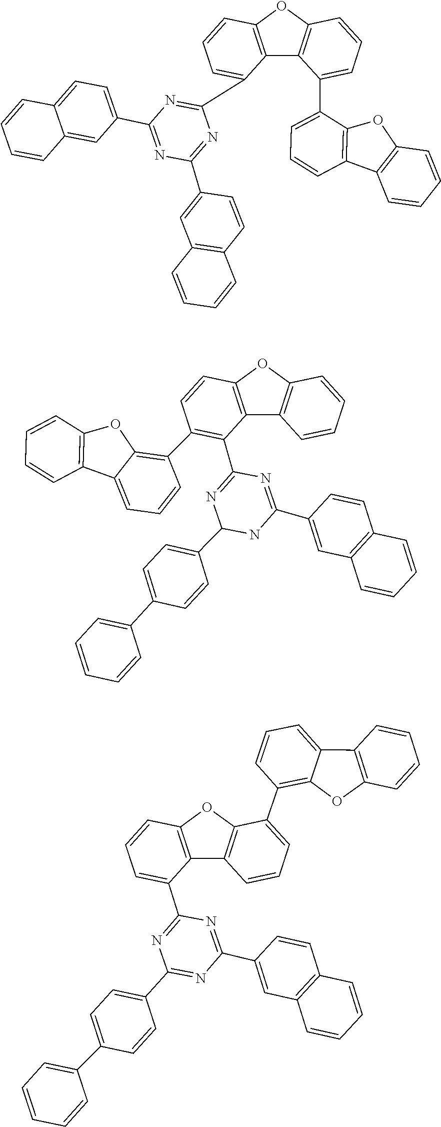

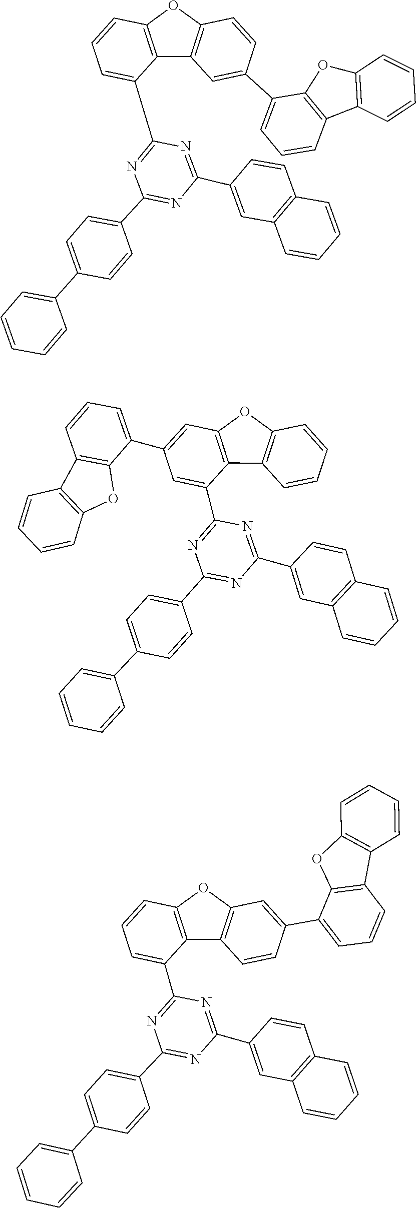 [SMILES: c1ccc(-c2ccc(-c3nc(-c4ccc5ccccc5c4)nc(-c4cccc5oc6ccc(-c7cccc8c7oc7ccccc78)cc6c45)n3)cc2)cc1 ; c1ccc(-c2ccc(-c3nc(-c4ccc5ccccc5c4)nc(-c4cc(-c5cccc6c5oc5ccccc56)cc5oc6ccccc6c45)n3)cc2)cc1 ; c1ccc(-c2ccc(-c3nc(-c4ccc5ccccc5c4)nc(-c4cccc5oc6cc(-c7cccc8c7oc7ccccc78)ccc6c45)n3)cc2)cc1]

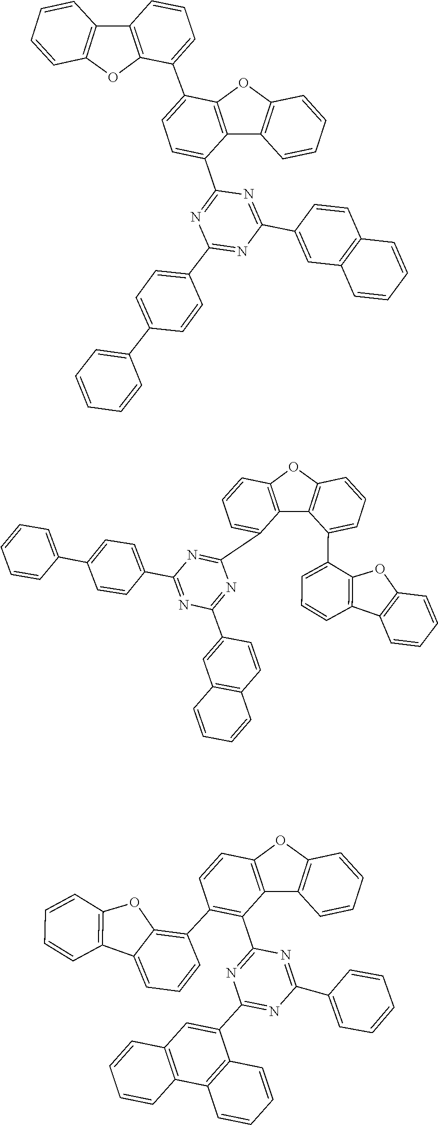 [SMILES: c1ccc(-c2ccc(-c3nc(-c4ccc5ccccc5c4)nc(-c4ccc(-c5cccc6c5oc5ccccc56)c5oc6ccccc6c45)n3)cc2)cc1 ; c1ccc(-c2ccc(-c3nc(-c4ccc5ccccc5c4)nc(-c4cccc5oc6cccc(-c7cccc8c7oc7ccccc78)c6c45)n3)cc2)cc1 ; c1ccc(-c2nc(-c3cc4ccccc4c4ccccc34)nc(-c3c(-c4cccc5c4oc4ccccc45)ccc4oc5ccccc5c34)n2)cc1]

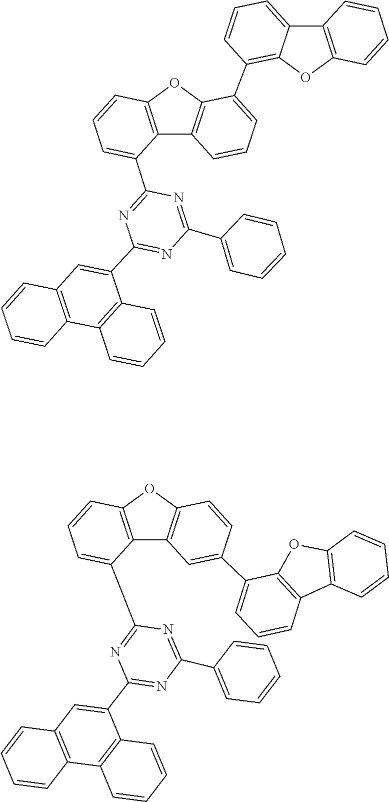 [SMILES: c1ccc(-c2nc(-c3cc4ccccc4c4ccccc34)nc(-c3cccc4oc5c(-c6cccc7c6oc6ccccc67)cccc5c34)n2)cc1 ; c1ccc(-c2nc(-c3cc4ccccc4c4ccccc34)nc(-c3cccc4oc5ccc(-c6cccc7c6oc6ccccc67)cc5c34)n2)cc1]

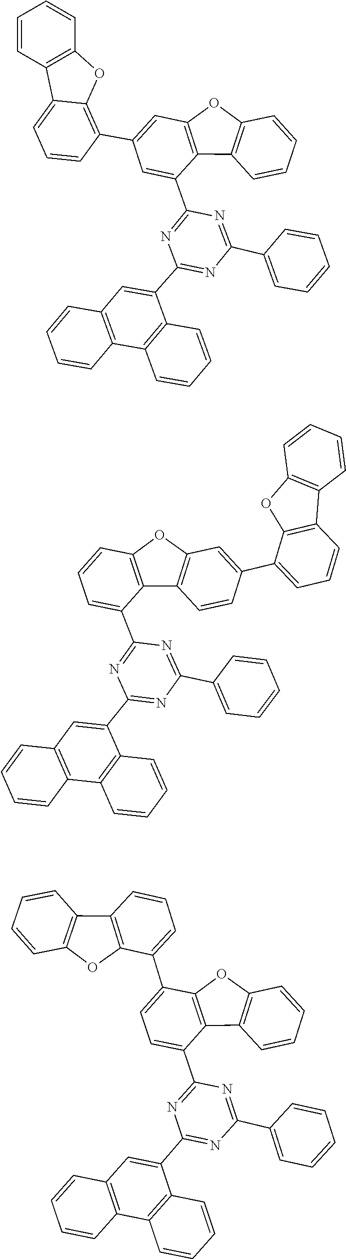 [SMILES: c1ccc(-c2nc(-c3cc4ccccc4c4ccccc34)nc(-c3cc(-c4cccc5c4oc4ccccc45)cc4oc5ccccc5c34)n2)cc1 ; c1ccc(-c2nc(-c3cc4ccccc4c4ccccc34)nc(-c3cccc4oc5cc(-c6cccc7c6oc6ccccc67)ccc5c34)n2)cc1 ; c1ccc(-c2nc(-c3cc4ccccc4c4ccccc34)nc(-c3ccc(-c4cccc5c4oc4ccccc45)c4oc5ccccc5c34)n2)cc1]

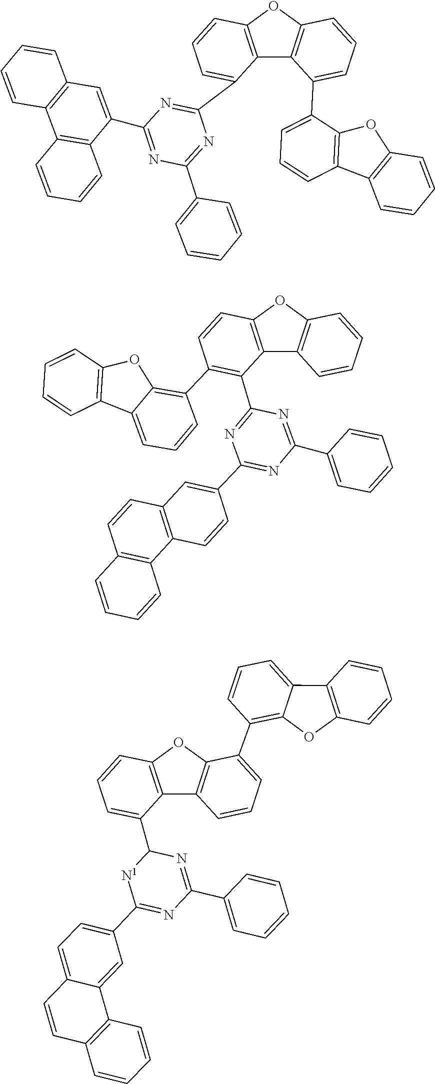

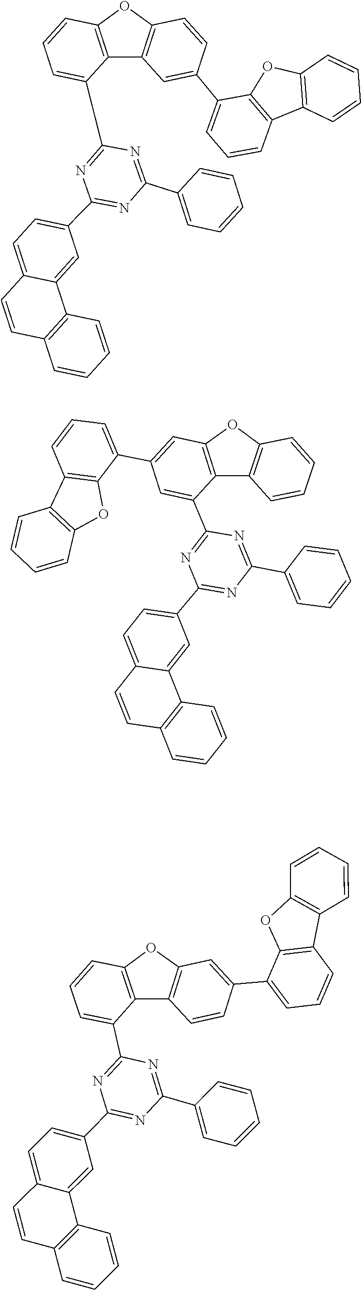

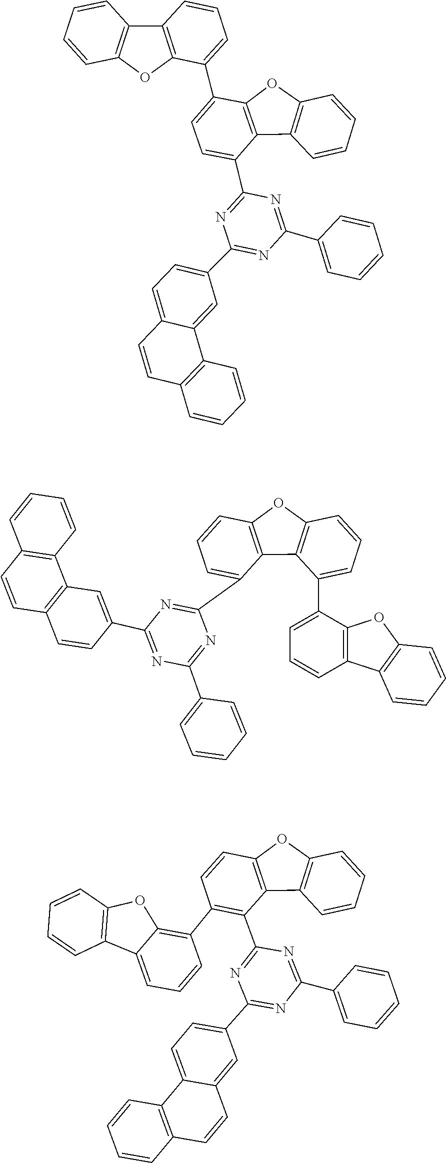 [SMILES: c1ccc(-c2nc(-c3ccc4ccc5ccccc5c4c3)nc(-c3ccc(-c4cccc5c4oc4ccccc45)c4oc5ccccc5c34)n2)cc1 ; c1ccc(-c2nc(-c3ccc4ccc5ccccc5c4c3)nc(-c3cccc4oc5cccc(-c6cccc7c6oc6ccccc67)c5c34)n2)cc1 ; c1ccc(-c2nc(-c3ccc4c(ccc5ccccc54)c3)nc(-c3c(-c4cccc5c4oc4ccccc45)ccc4oc5ccccc5c34)n2)cc1]

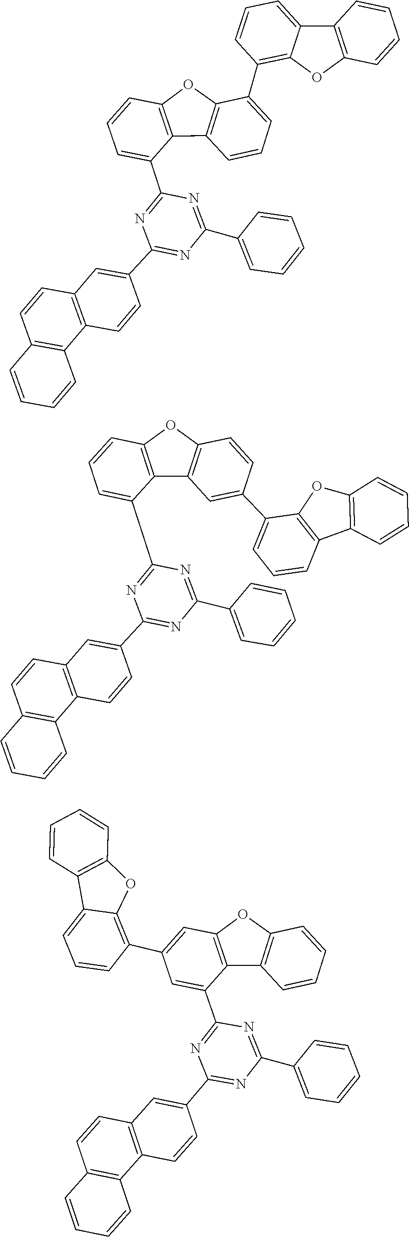 [SMILES: c1ccc(-c2nc(-c3ccc4c(ccc5ccccc54)c3)nc(-c3cccc4oc5c(-c6cccc7c6oc6ccccc67)cccc5c34)n2)cc1 ; c1ccc(-c2nc(-c3ccc4c(ccc5ccccc54)c3)nc(-c3cccc4oc5ccc(-c6cccc7c6oc6ccccc67)cc5c34)n2)cc1 ; c1ccc(-c2nc(-c3ccc4c(ccc5ccccc54)c3)nc(-c3cc(-c4cccc5c4oc4ccccc45)cc4oc5ccccc5c34)n2)cc1]

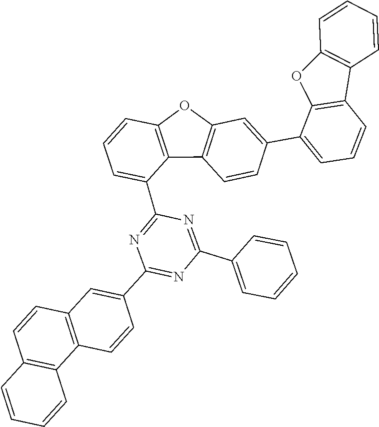 [SMILES: c1ccc(-c2nc(-c3ccc4c(ccc5ccccc54)c3)nc(-c3cccc4oc5cc(-c6cccc7c6oc6ccccc67)ccc5c34)n2)cc1]

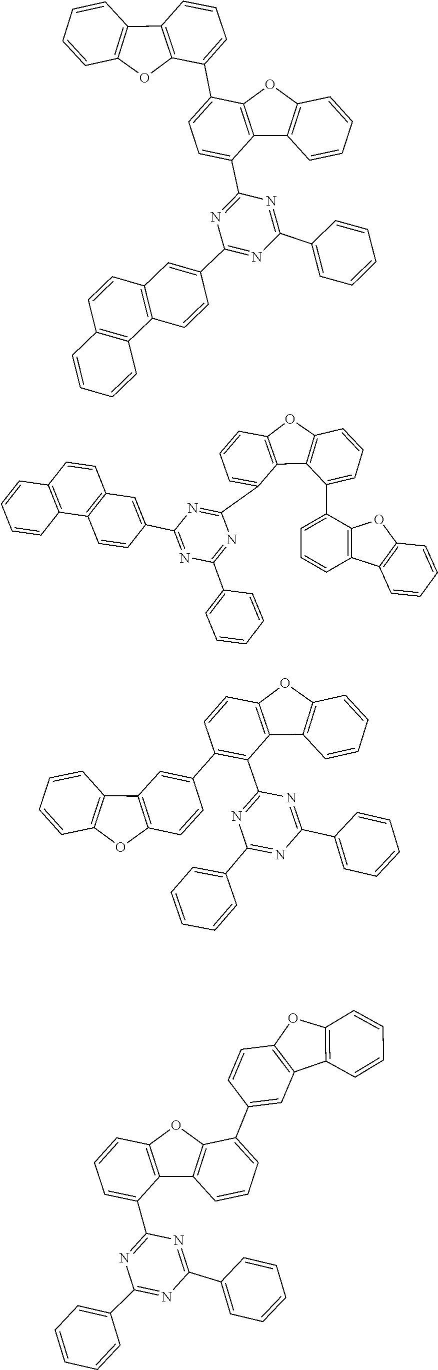 [SMILES: c1ccc(-c2nc(-c3ccc4c(ccc5ccccc54)c3)nc(-c3ccc(-c4cccc5c4oc4ccccc45)c4oc5ccccc5c34)n2)cc1 ; c1ccc(-c2nc(-c3ccc4c(ccc5ccccc54)c3)nc(-c3cccc4oc5cccc(-c6cccc7c6oc6ccccc67)c5c34)n2)cc1 ; c1ccc(-c2nc(-c3ccccc3)nc(-c3c(-c4ccc5oc6ccccc6c5c4)ccc4oc5ccccc5c34)n2)cc1 ; c1ccc(-c2nc(-c3ccccc3)nc(-c3cccc4oc5c(-c6ccc7oc8ccccc8c7c6)cccc5c34)n2)cc1]

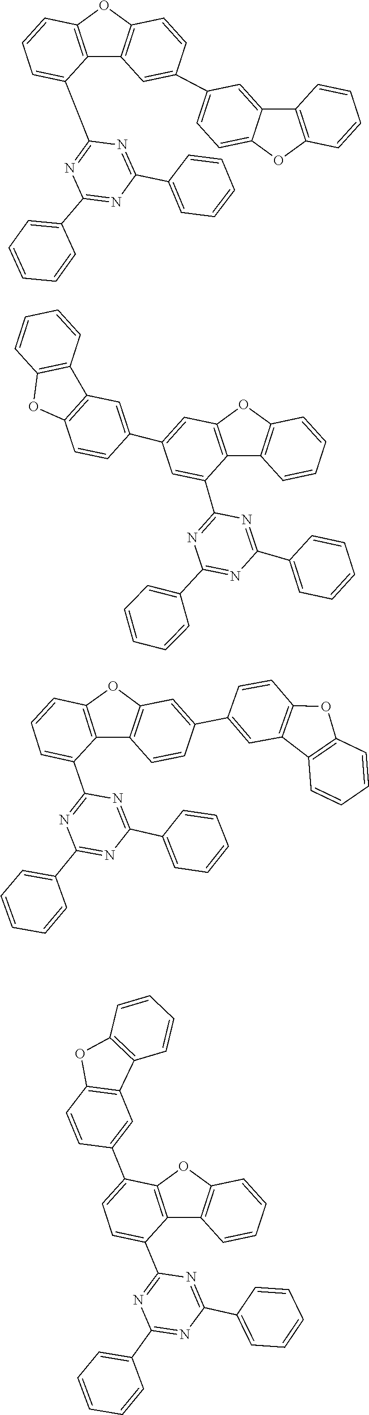

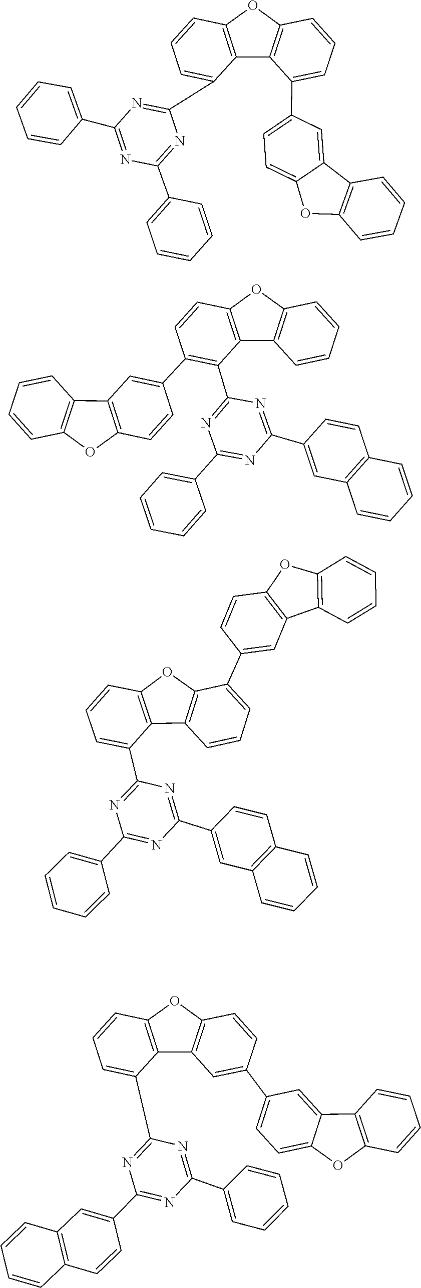

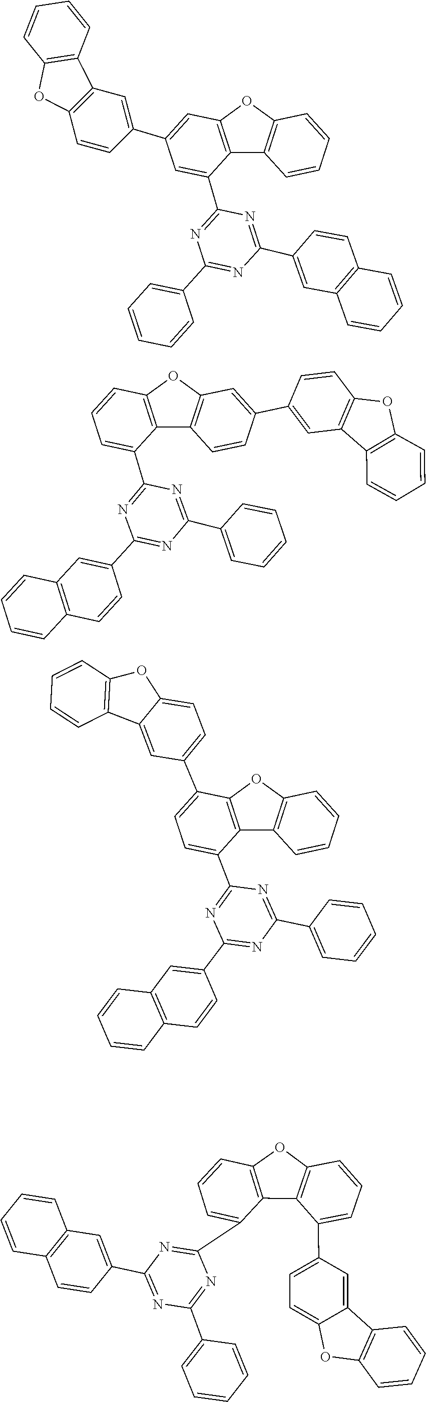

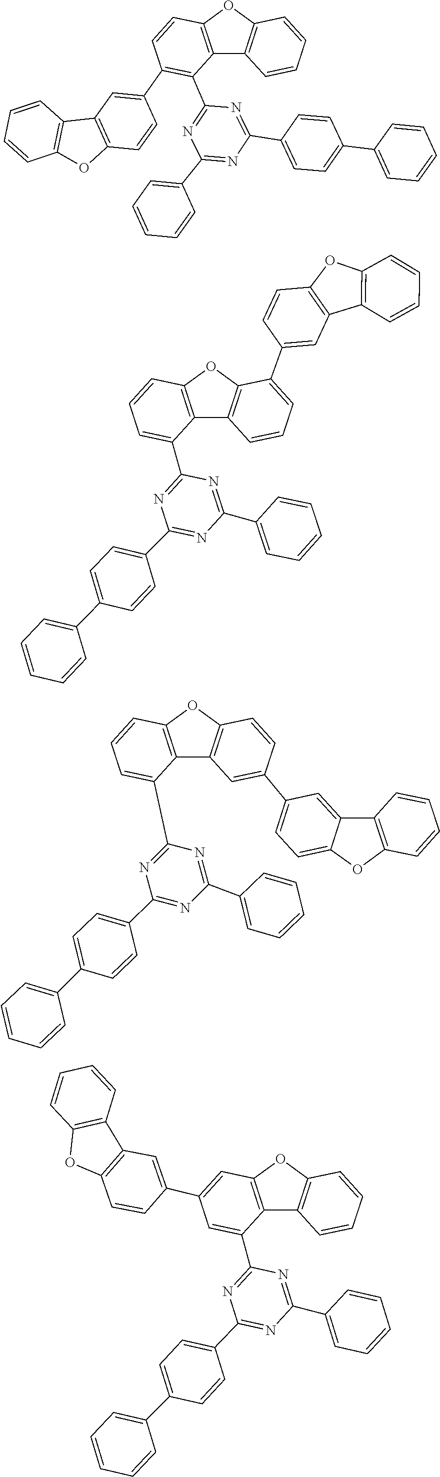

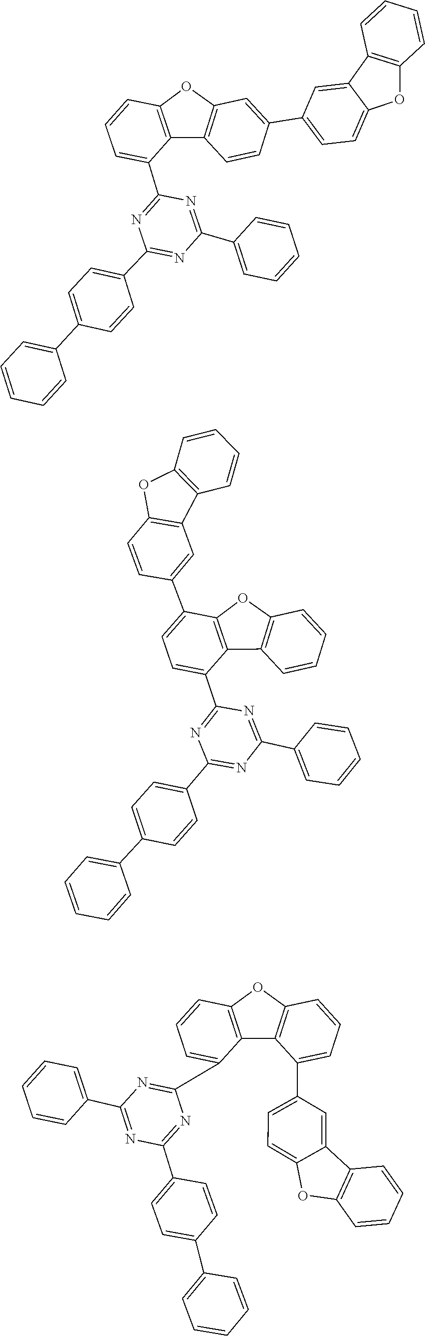 [SMILES: c1ccc(-c2ccc(-c3nc(-c4ccccc4)nc(-c4cccc5oc6cc(-c7ccc8oc9ccccc9c8c7)ccc6c45)n3)cc2)cc1 ; c1ccc(-c2ccc(-c3nc(-c4ccccc4)nc(-c4ccc(-c5ccc6oc7ccccc7c6c5)c5oc6ccccc6c45)n3)cc2)cc1 ; c1ccc(-c2ccc(-c3nc(-c4ccccc4)nc(-c4cccc5oc6cccc(-c7ccc8oc9ccccc9c8c7)c6c45)n3)cc2)cc1]

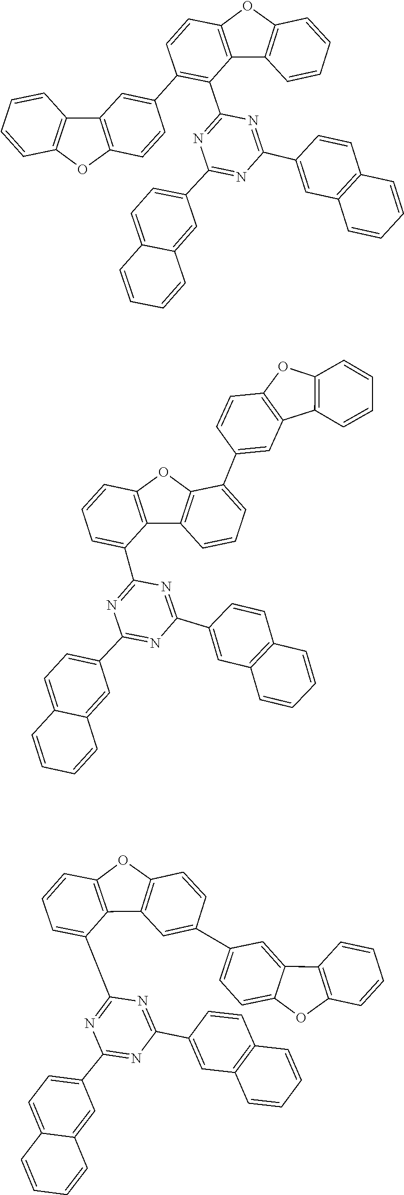 [SMILES: c1ccc2cc(-c3nc(-c4ccc5ccccc5c4)nc(-c4c(-c5ccc6oc7ccccc7c6c5)ccc5oc6ccccc6c45)n3)ccc2c1 ; c1ccc2cc(-c3nc(-c4ccc5ccccc5c4)nc(-c4cccc5oc6c(-c7ccc8oc9ccccc9c8c7)cccc6c45)n3)ccc2c1 ; c1ccc2cc(-c3nc(-c4ccc5ccccc5c4)nc(-c4cccc5oc6ccc(-c7ccc8oc9ccccc9c8c7)cc6c45)n3)ccc2c1]

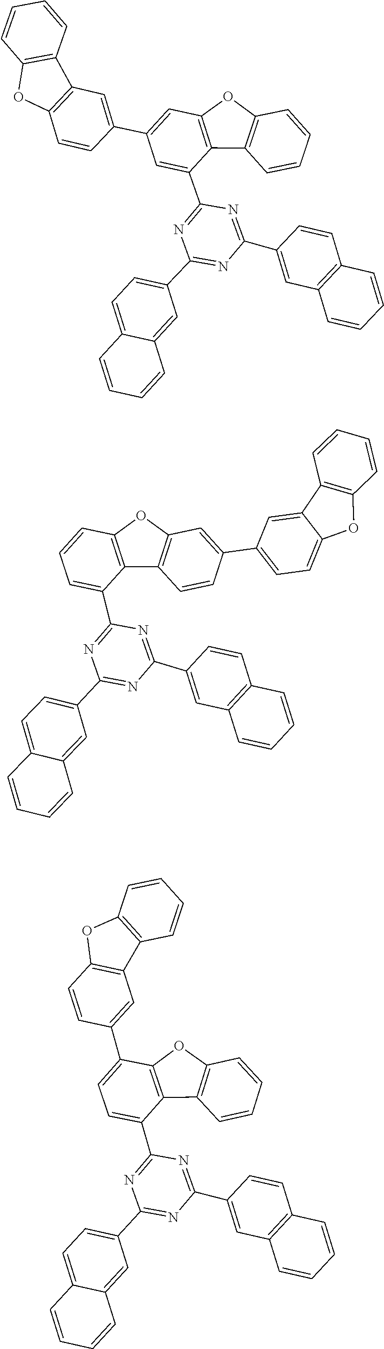 [SMILES: c1ccc2cc(-c3nc(-c4ccc5ccccc5c4)nc(-c4cc(-c5ccc6oc7ccccc7c6c5)cc5oc6ccccc6c45)n3)ccc2c1 ; c1ccc2cc(-c3nc(-c4ccc5ccccc5c4)nc(-c4cccc5oc6cc(-c7ccc8oc9ccccc9c8c7)ccc6c45)n3)ccc2c1 ; c1ccc2cc(-c3nc(-c4ccc5ccccc5c4)nc(-c4ccc(-c5ccc6oc7ccccc7c6c5)c5oc6ccccc6c45)n3)ccc2c1]

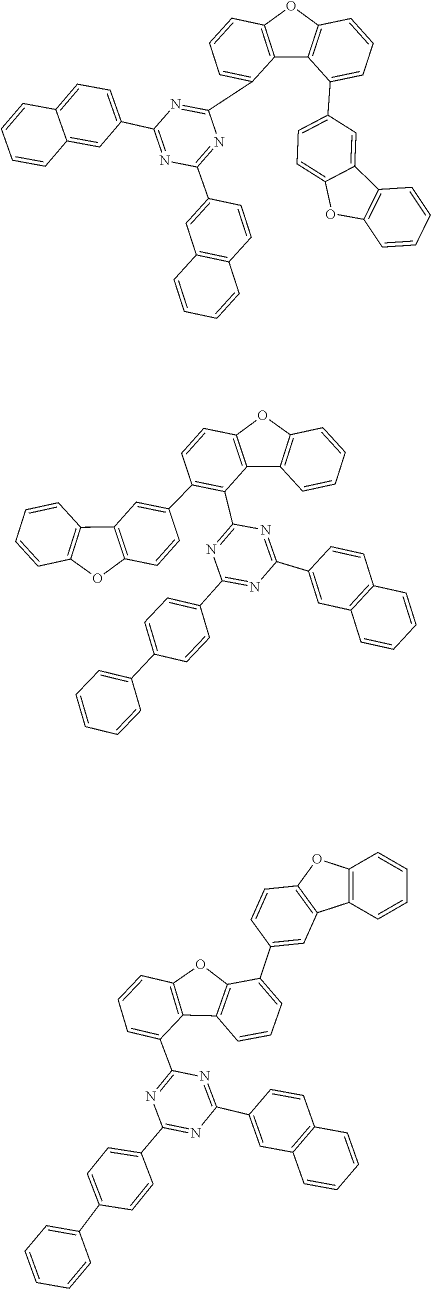 [SMILES: c1ccc2cc(-c3nc(-c4ccc5ccccc5c4)nc(-c4cccc5oc6cccc(-c7ccc8oc9ccccc9c8c7)c6c45)n3)ccc2c1 ; c1ccc(-c2ccc(-c3nc(-c4ccc5ccccc5c4)nc(-c4c(-c5ccc6oc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7ccc8oc9ccccc9c8c7)cccc6c45)n3)cc2)cc1]

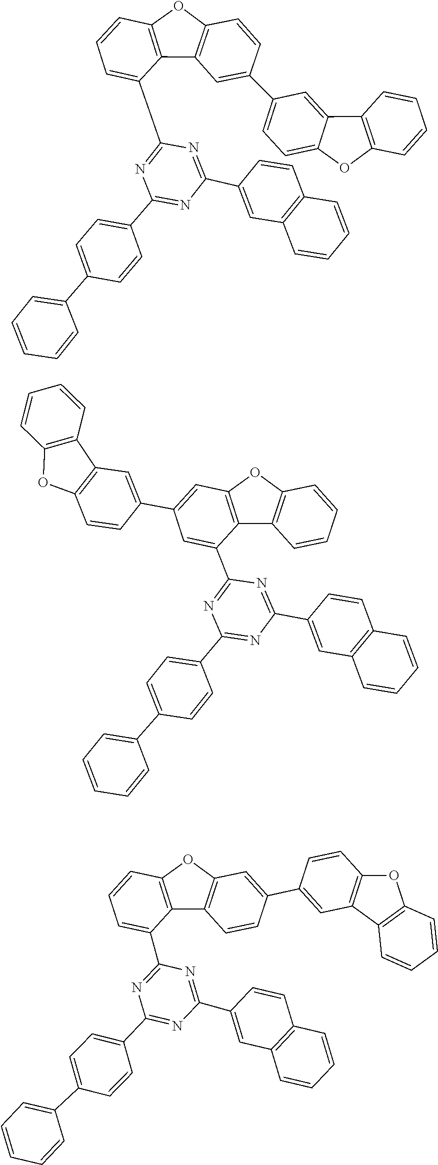

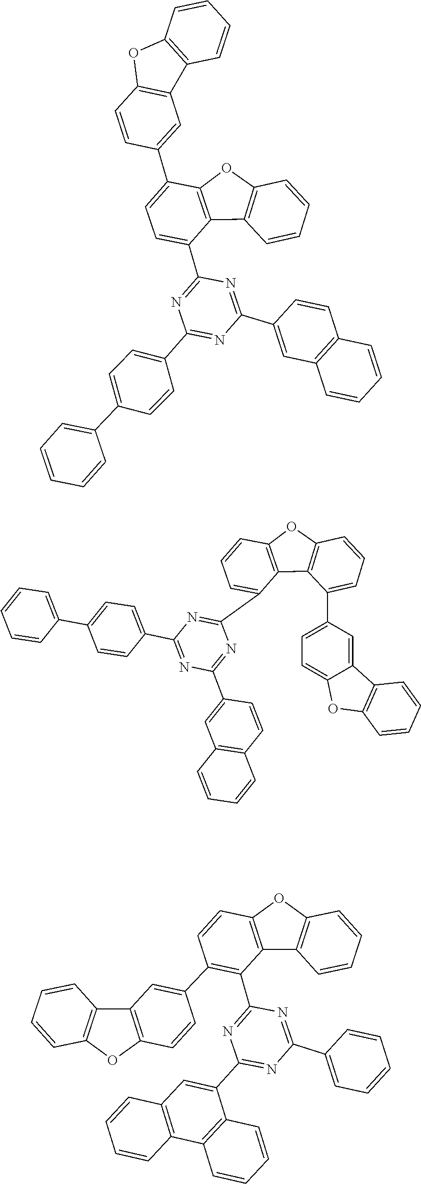 [SMILES: c1ccc(-c2ccc(-c3nc(-c4ccc5ccccc5c4)nc(-c4ccc(-c5ccc6oc7ccccc7c6c5)c5oc6ccccc6c45)n3)cc2)cc1 ; c1ccc(-c2ccc(-c3nc(-c4ccc5ccccc5c4)nc(-c4cccc5oc6cccc(-c7ccc8oc9ccccc9c8c7)c6c45)n3)cc2)cc1 ; c1ccc(-c2nc(-c3cc4ccccc4c4ccccc34)nc(-c3c(-c4ccc5oc6ccccc6c5c4)ccc4oc5ccccc5c34)n2)cc1]

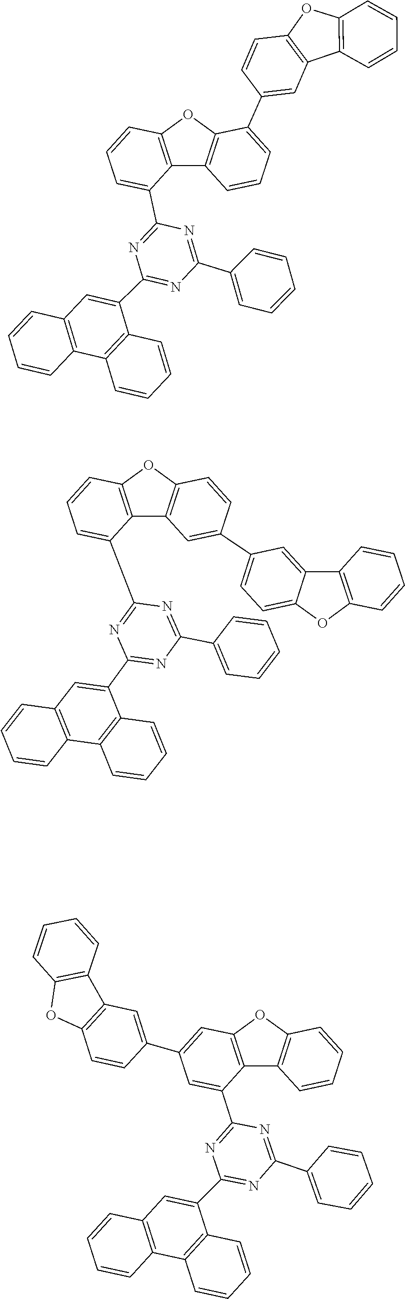 [SMILES: c1ccc(-c2nc(-c3cc4ccccc4c4ccccc34)nc(-c3cccc4oc5c(-c6ccc7oc8ccccc8c7c6)cccc5c34)n2)cc1 ; c1ccc(-c2nc(-c3cc4ccccc4c4ccccc34)nc(-c3cccc4oc5ccc(-c6ccc7oc8ccccc8c7c6)cc5c34)n2)cc1 ; c1ccc(-c2nc(-c3cc4ccccc4c4ccccc34)nc(-c3cc(-c4ccc5oc6ccccc6c5c4)cc4oc5ccccc5c34)n2)cc1]

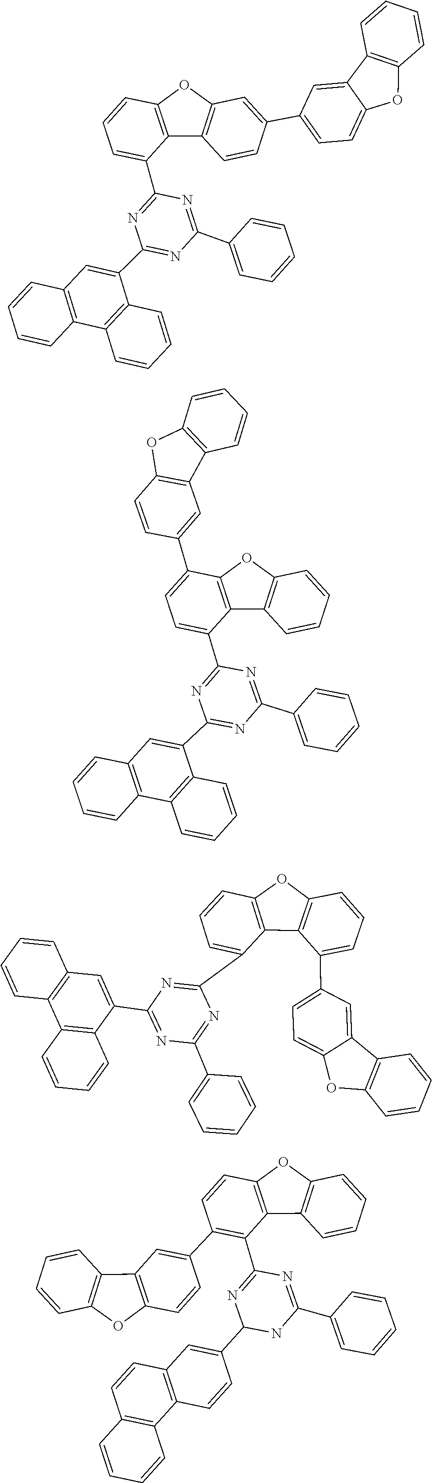

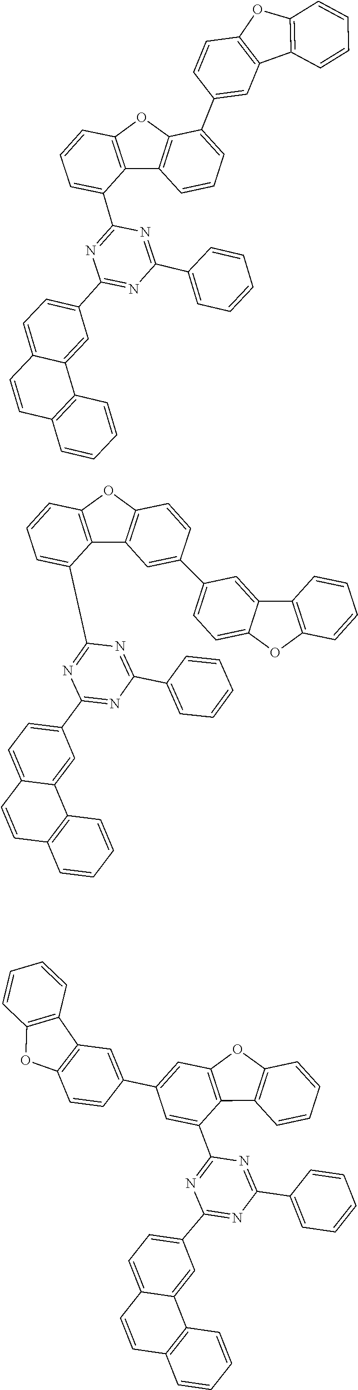

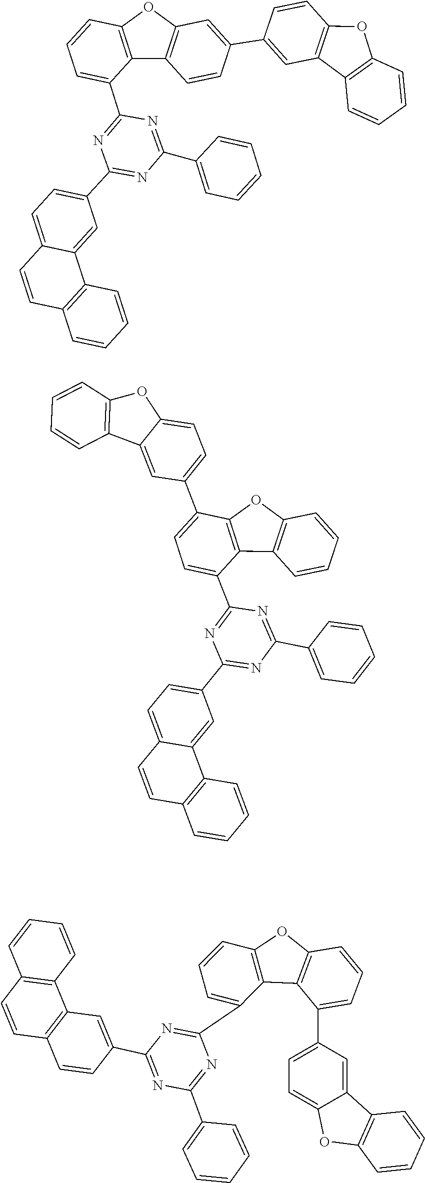

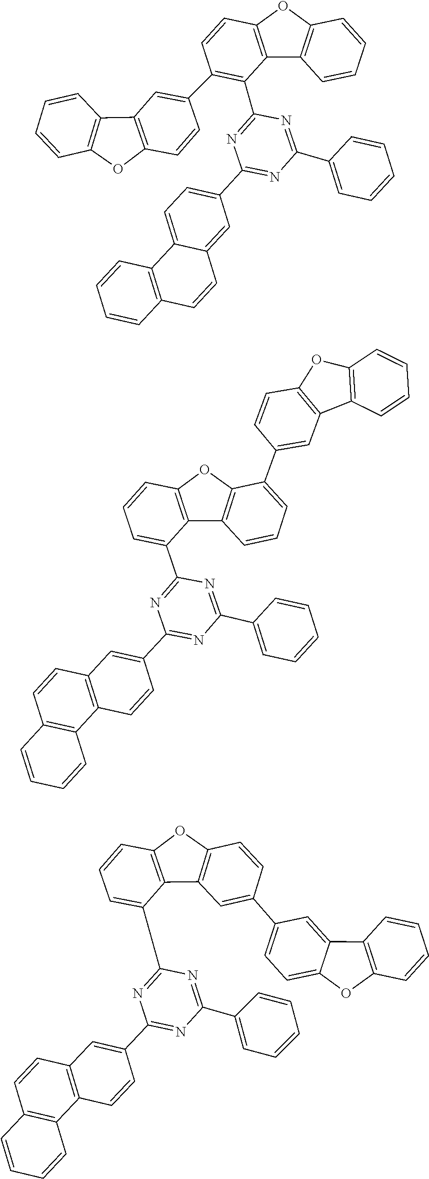 [SMILES: c1ccc(-c2nc(-c3ccc4c(ccc5ccccc54)c3)nc(-c3c(-c4ccc5oc6ccccc6c5c4)ccc4oc5ccccc5c34)n2)cc1 ; c1ccc(-c2nc(-c3ccc4c(ccc5ccccc54)c3)nc(-c3cccc4oc5c(-c6ccc7oc8ccccc8c7c6)cccc5c34)n2)cc1 ; c1ccc(-c2nc(-c3ccc4c(ccc5ccccc54)c3)nc(-c3cccc4oc5ccc(-c6ccc7oc8ccccc8c7c6)cc5c34)n2)cc1]

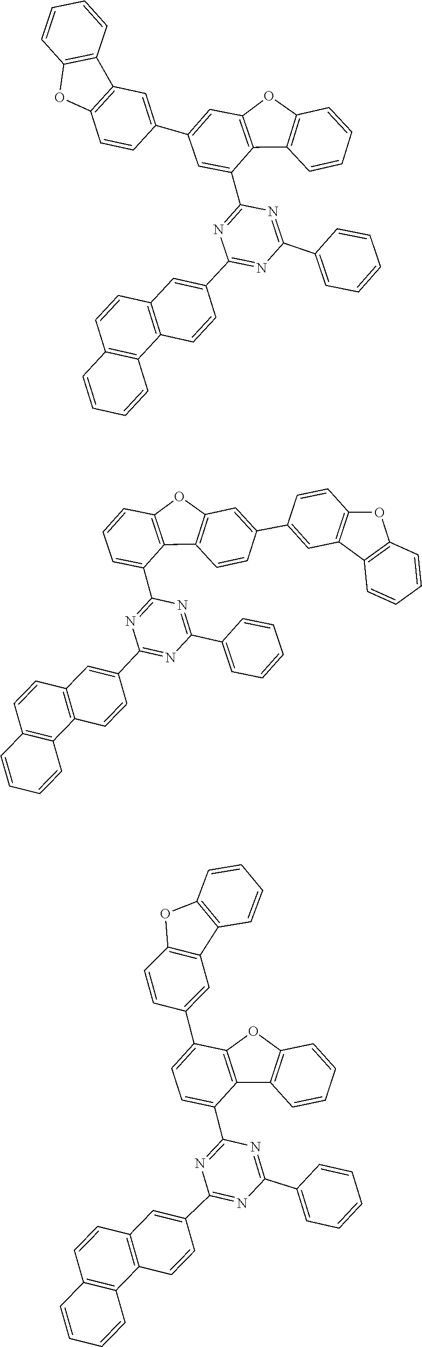 [SMILES: c1ccc(-c2nc(-c3ccc4c(ccc5ccccc54)c3)nc(-c3cc(-c4ccc5oc6ccccc6c5c4)cc4oc5ccccc5c34)n2)cc1 ; c1ccc(-c2nc(-c3ccc4c(ccc5ccccc54)c3)nc(-c3cccc4oc5cc(-c6ccc7oc8ccccc8c7c6)ccc5c34)n2)cc1 ; c1ccc(-c2nc(-c3ccc4c(ccc5ccccc54)c3)nc(-c3ccc(-c4ccc5oc6ccccc6c5c4)c4oc5ccccc5c34)n2)cc1]

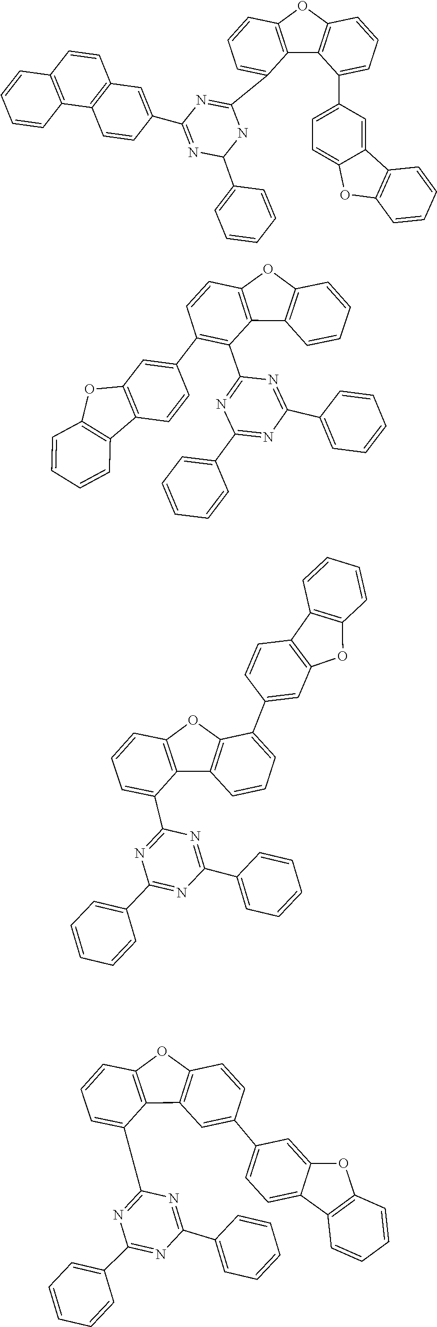

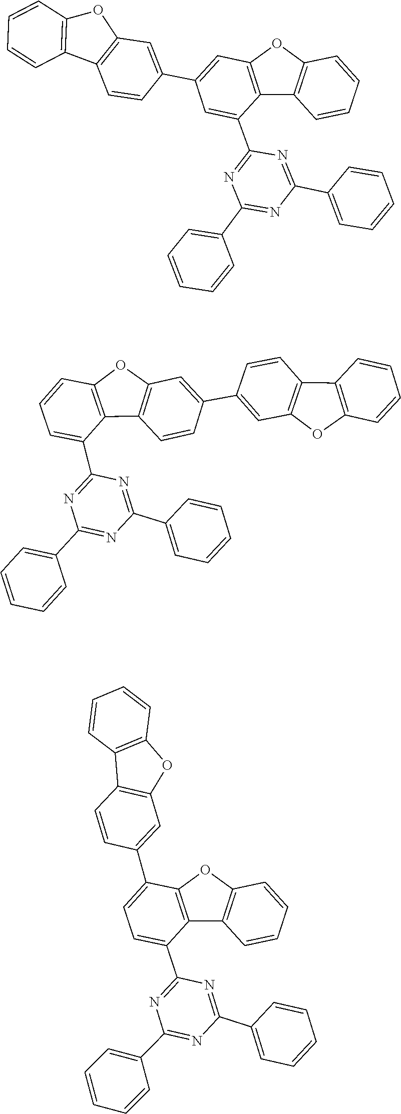 [SMILES: c1ccc(-c2nc(-c3ccccc3)nc(-c3cc(-c4ccc5c(c4)oc4ccccc45)cc4oc5ccccc5c34)n2)cc1 ; c1ccc(-c2nc(-c3ccccc3)nc(-c3cccc4oc5cc(-c6ccc7c(c6)oc6ccccc67)ccc5c34)n2)cc1 ; c1ccc(-c2nc(-c3ccccc3)nc(-c3ccc(-c4ccc5c(c4)oc4ccccc45)c4oc5ccccc5c34)n2)cc1]

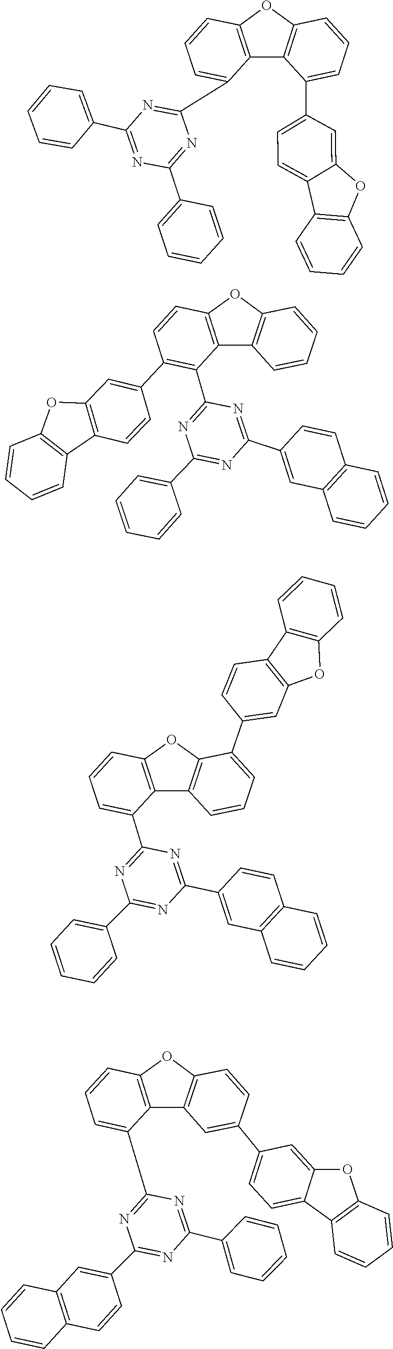

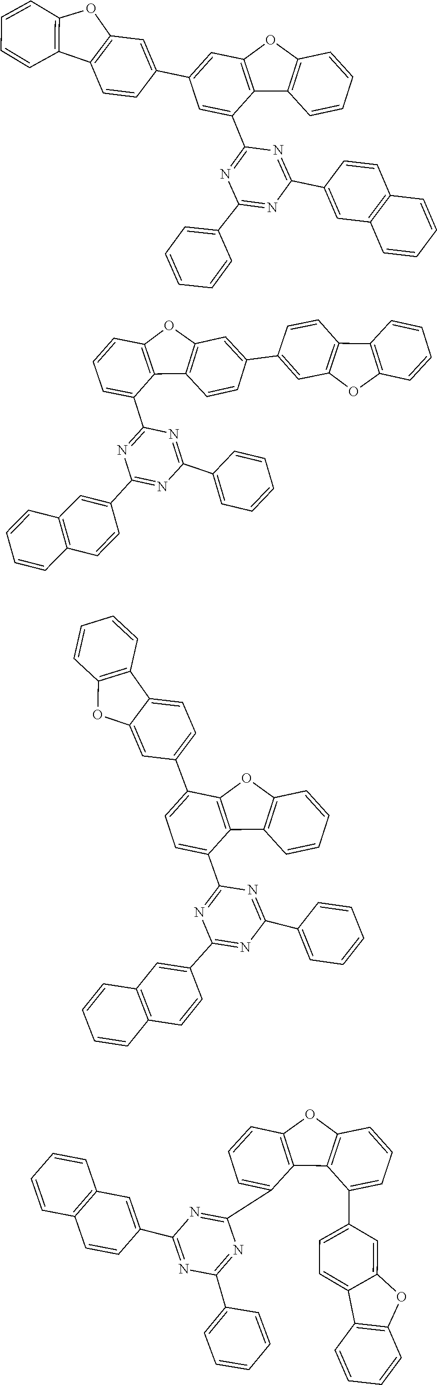

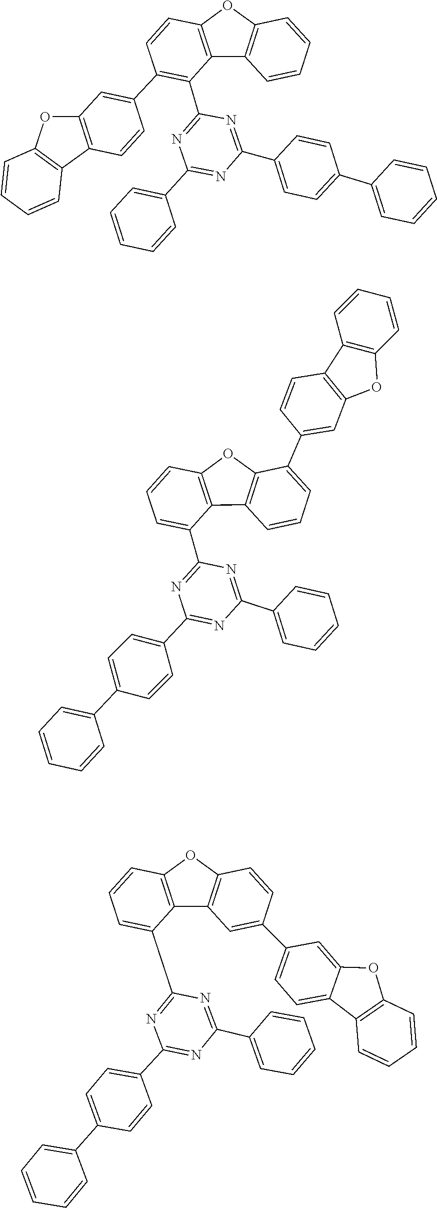 [SMILES: c1ccc(-c2ccc(-c3nc(-c4ccccc4)nc(-c4c(-c5ccc6c(c5)oc5ccccc56)ccc5oc6ccccc6c45)n3)cc2)cc1 ; c1ccc(-c2ccc(-c3nc(-c4ccccc4)nc(-c4cccc5oc6c(-c7ccc8c(c7)oc7ccccc78)cccc6c45)n3)cc2)cc1 ; c1ccc(-c2ccc(-c3nc(-c4ccccc4)nc(-c4cccc5oc6ccc(-c7ccc8c(c7)oc7ccccc78)cc6c45)n3)cc2)cc1]

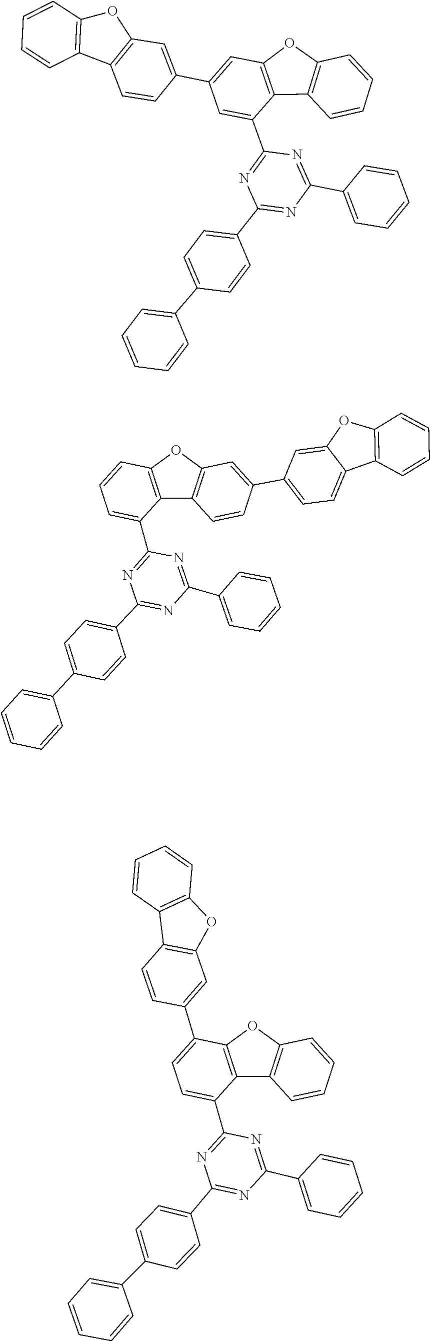 [SMILES: c1ccc(-c2ccc(-c3nc(-c4ccccc4)nc(-c4cc(-c5ccc6c(c5)oc5ccccc56)cc5oc6ccccc6c45)n3)cc2)cc1 ; c1ccc(-c2ccc(-c3nc(-c4ccccc4)nc(-c4cccc5oc6cc(-c7ccc8c(c7)oc7ccccc78)ccc6c45)n3)cc2)cc1 ; c1ccc(-c2ccc(-c3nc(-c4ccccc4)nc(-c4ccc(-c5ccc6c(c5)oc5ccccc56)c5oc6ccccc6c45)n3)cc2)cc1]

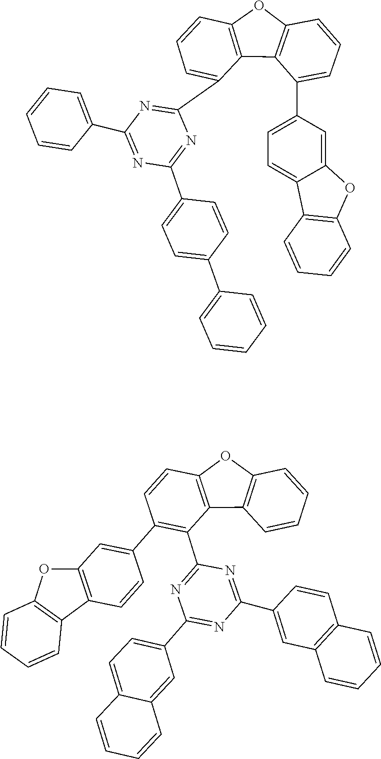 [SMILES: c1ccc(-c2ccc(-c3nc(-c4ccccc4)nc(-c4cccc5oc6cccc(-c7ccc8c(c7)oc7ccccc78)c6c45)n3)cc2)cc1 ; c1ccc2cc(-c3nc(-c4ccc5ccccc5c4)nc(-c4c(-c5ccc6c(c5)oc5ccccc56)ccc5oc6ccccc6c45)n3)ccc2c1]

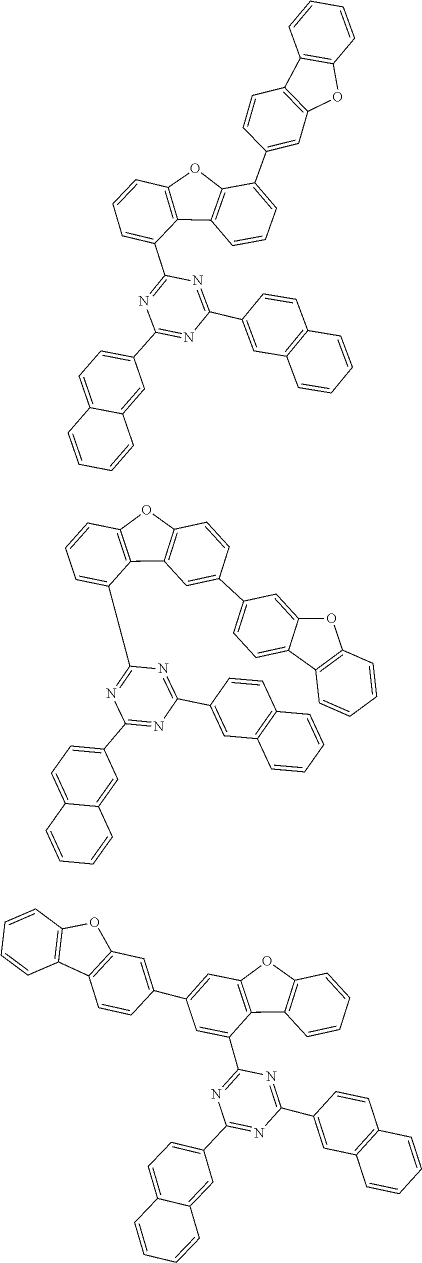 [SMILES: c1ccc2cc(-c3nc(-c4ccc5ccccc5c4)nc(-c4cccc5oc6c(-c7ccc8c(c7)oc7ccccc78)cccc6c45)n3)ccc2c1 ; c1ccc2cc(-c3nc(-c4ccc5ccccc5c4)nc(-c4cccc5oc6ccc(-c7ccc8c(c7)oc7ccccc78)cc6c45)n3)ccc2c1 ; c1ccc2cc(-c3nc(-c4ccc5ccccc5c4)nc(-c4cc(-c5ccc6c(c5)oc5ccccc56)cc5oc6ccccc6c45)n3)ccc2c1]

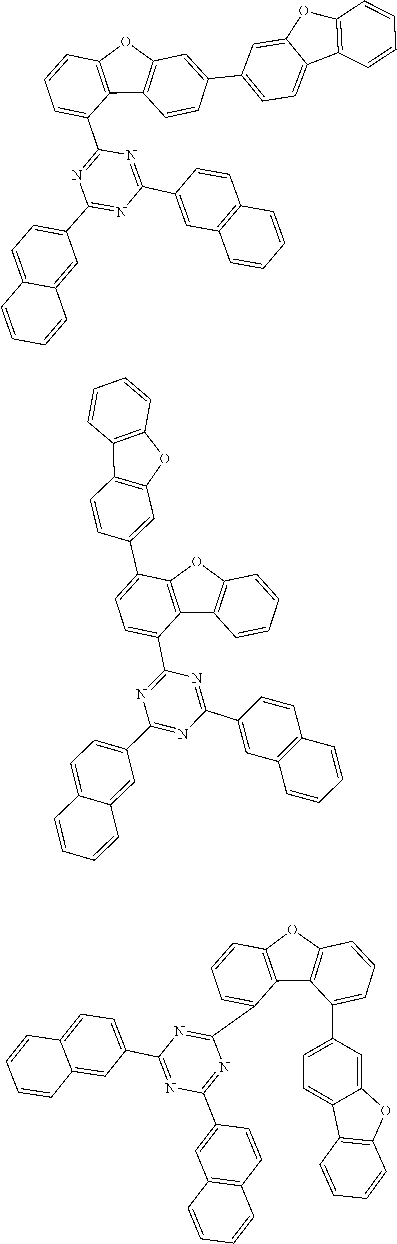 [SMILES: c1ccc2cc(-c3nc(-c4ccc5ccccc5c4)nc(-c4cccc5oc6cc(-c7ccc8c(c7)oc7ccccc78)ccc6c45)n3)ccc2c1 ; c1ccc2cc(-c3nc(-c4ccc5ccccc5c4)nc(-c4ccc(-c5ccc6c(c5)oc5ccccc56)c5oc6ccccc6c45)n3)ccc2c1 ; c1ccc2cc(-c3nc(-c4ccc5ccccc5c4)nc(-c4cccc5oc6cccc(-c7ccc8c(c7)oc7ccccc78)c6c45)n3)ccc2c1]

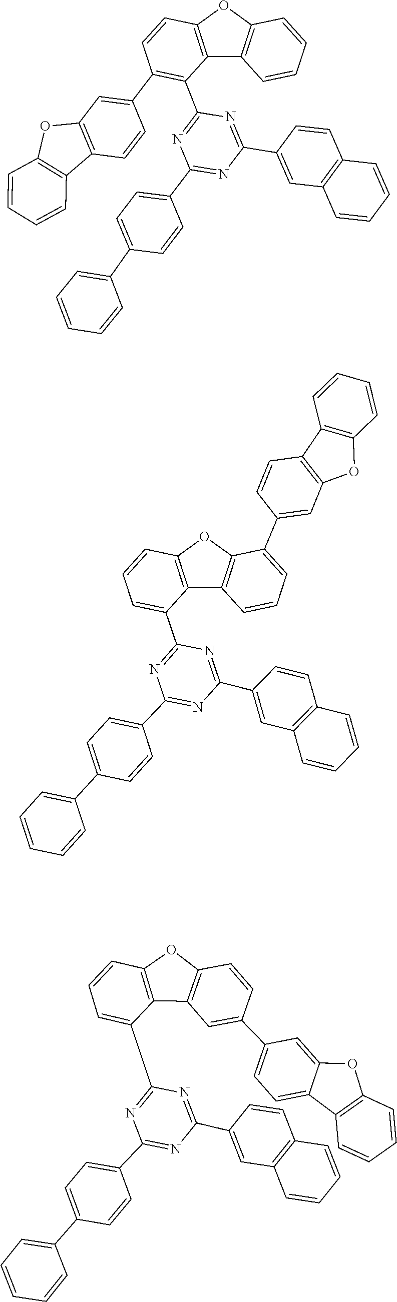 [SMILES: c1ccc(-c2ccc(-c3nc(-c4ccc5ccccc5c4)nc(-c4c(-c5ccc6c(c5)oc5ccccc56)ccc5oc6ccccc6c45)n3)cc2)cc1 ; c1ccc(-c2ccc(-c3nc(-c4ccc5ccccc5c4)nc(-c4cccc5oc6c(-c7ccc8c(c7)oc7ccccc78)cccc6c45)n3)cc2)cc1 ; c1ccc(-c2ccc(-c3nc(-c4ccc5ccccc5c4)nc(-c4cccc5oc6ccc(-c7ccc8c(c7)oc7ccccc78)cc6c45)n3)cc2)cc1]

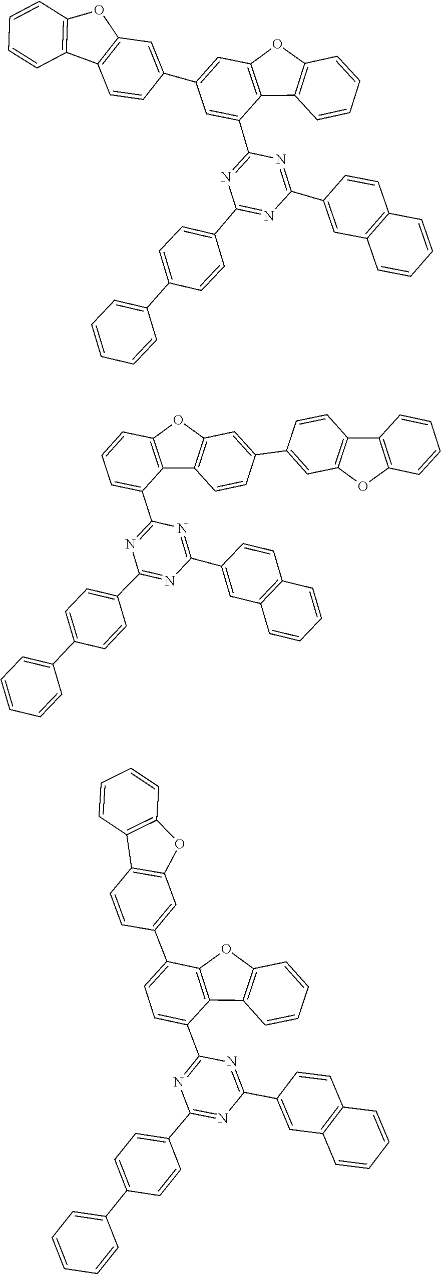 [SMILES: c1ccc(-c2ccc(-c3nc(-c4ccc5ccccc5c4)nc(-c4cc(-c5ccc6c(c5)oc5ccccc56)cc5oc6ccccc6c45)n3)cc2)cc1 ; c1ccc(-c2ccc(-c3nc(-c4ccc5ccccc5c4)nc(-c4cccc5oc6cc(-c7ccc8c(c7)oc7ccccc78)ccc6c45)n3)cc2)cc1 ; c1ccc(-c2ccc(-c3nc(-c4ccc5ccccc5c4)nc(-c4ccc(-c5ccc6c(c5)oc5ccccc56)c5oc6ccccc6c45)n3)cc2)cc1]

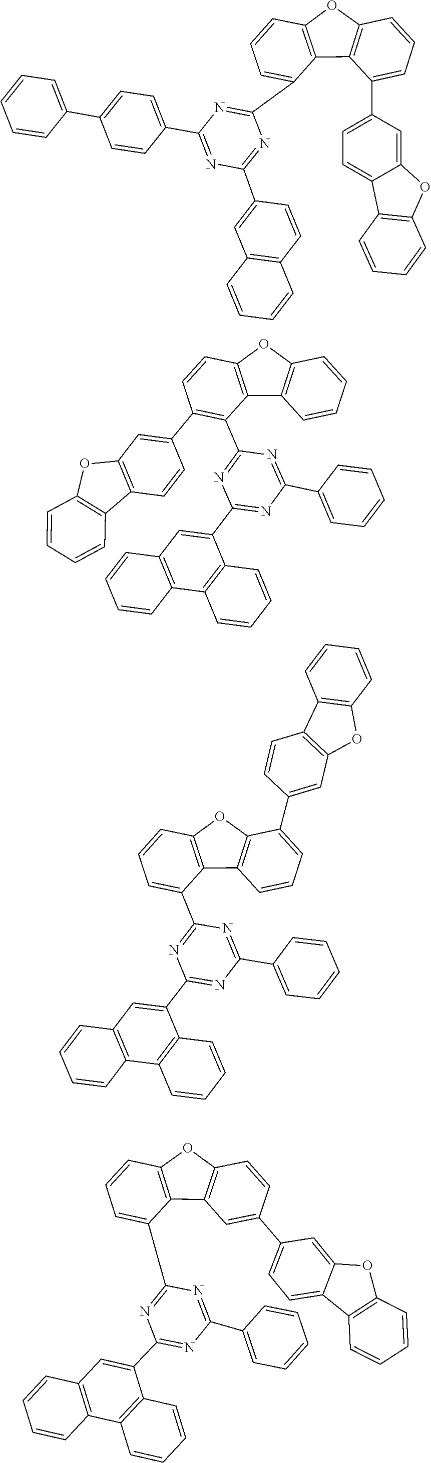

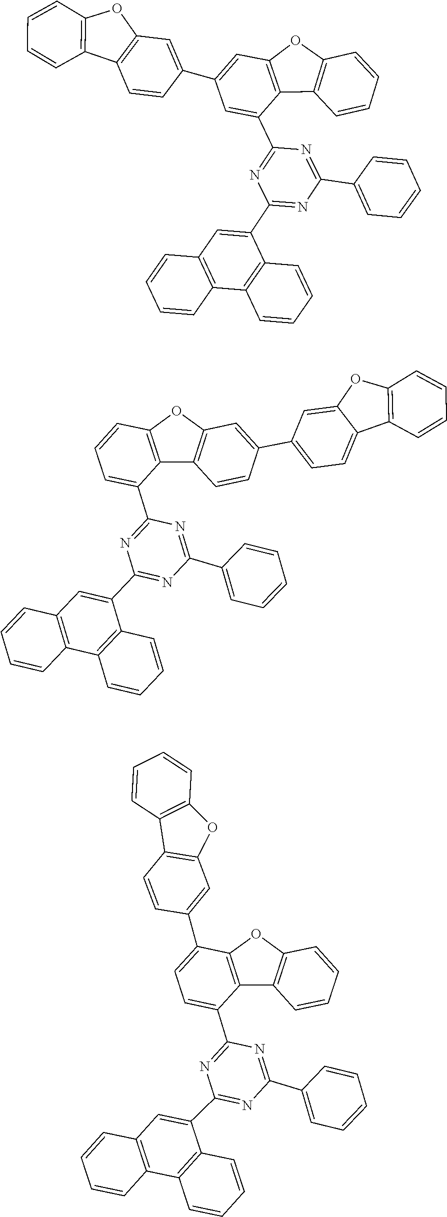 [SMILES: c1ccc(-c2nc(-c3cc4ccccc4c4ccccc34)nc(-c3cc(-c4ccc5c(c4)oc4ccccc45)cc4oc5ccccc5c34)n2)cc1 ; c1ccc(-c2nc(-c3cc4ccccc4c4ccccc34)nc(-c3cccc4oc5cc(-c6ccc7c(c6)oc6ccccc67)ccc5c34)n2)cc1 ; c1ccc(-c2nc(-c3cc4ccccc4c4ccccc34)nc(-c3ccc(-c4ccc5c(c4)oc4ccccc45)c4oc5ccccc5c34)n2)cc1]

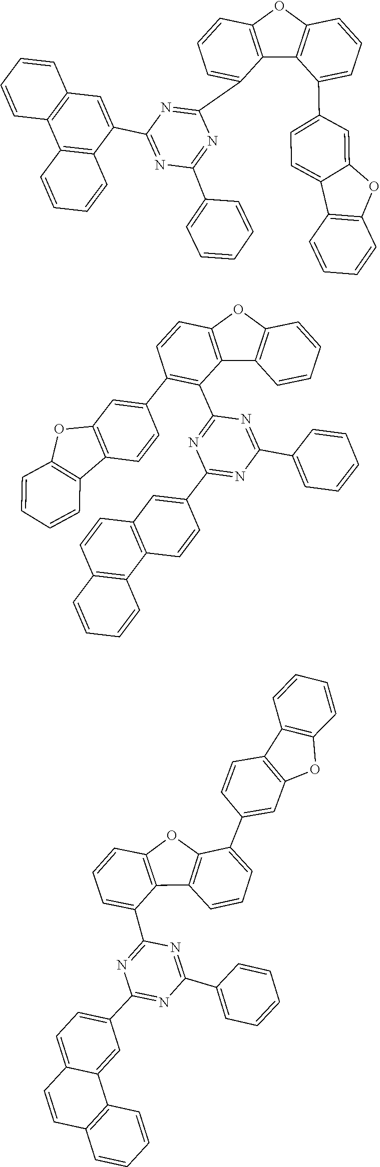 [SMILES: c1ccc(-c2nc(-c3cc4ccccc4c4ccccc34)nc(-c3cccc4oc5cccc(-c6ccc7c(c6)oc6ccccc67)c5c34)n2)cc1 ; c1ccc(-c2nc(-c3ccc4c(ccc5ccccc54)c3)nc(-c3c(-c4ccc5c(c4)oc4ccccc45)ccc4oc5ccccc5c34)n2)cc1 ; c1ccc(-c2nc(-c3ccc4ccc5ccccc5c4c3)nc(-c3cccc4oc5c(-c6ccc7c(c6)oc6ccccc67)cccc5c34)n2)cc1]

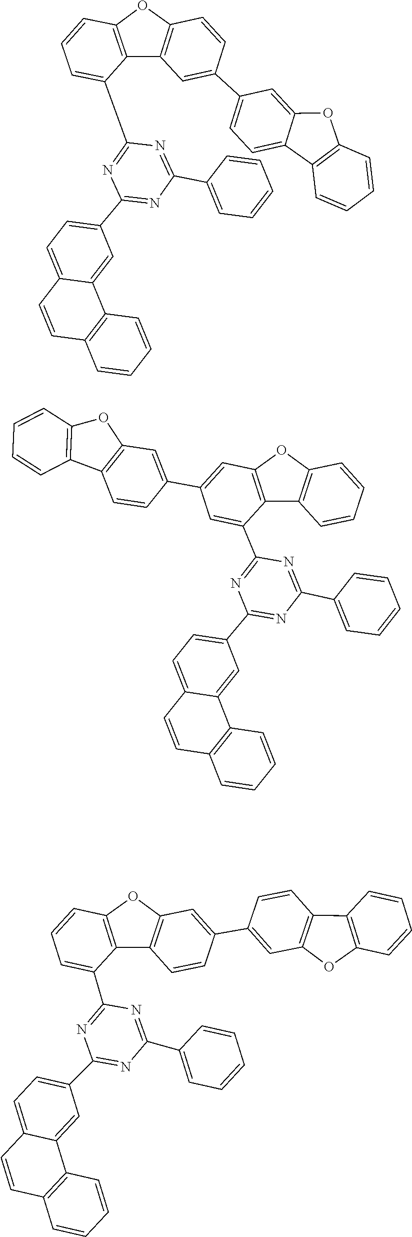 [SMILES: c1ccc(-c2nc(-c3ccc4ccc5ccccc5c4c3)nc(-c3cccc4oc5ccc(-c6ccc7c(c6)oc6ccccc67)cc5c34)n2)cc1 ; c1ccc(-c2nc(-c3ccc4ccc5ccccc5c4c3)nc(-c3cc(-c4ccc5c(c4)oc4ccccc45)cc4oc5ccccc5c34)n2)cc1 ; c1ccc(-c2nc(-c3ccc4ccc5ccccc5c4c3)nc(-c3cccc4oc5cc(-c6ccc7c(c6)oc6ccccc67)ccc5c34)n2)cc1]

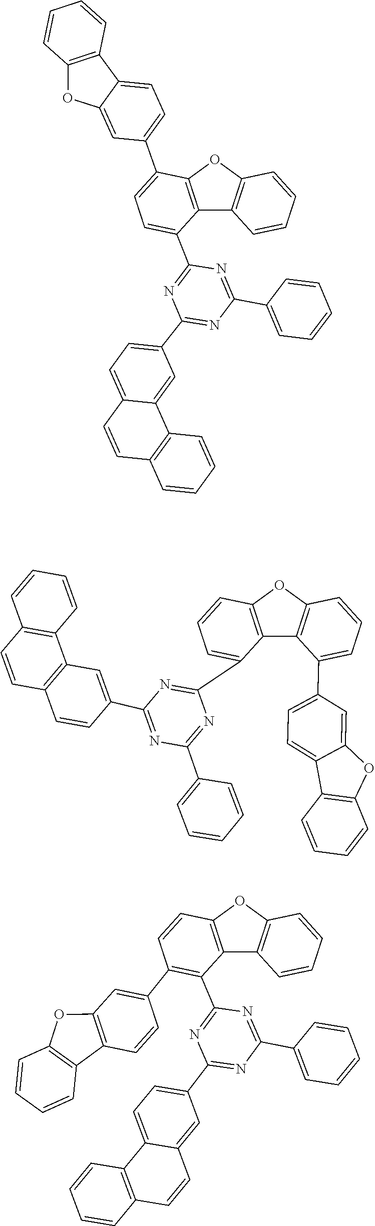 [SMILES: c1ccc(-c2nc(-c3ccc4ccc5ccccc5c4c3)nc(-c3ccc(-c4ccc5c(c4)oc4ccccc45)c4oc5ccccc5c34)n2)cc1 ; c1ccc(-c2nc(-c3ccc4ccc5ccccc5c4c3)nc(-c3cccc4oc5cccc(-c6ccc7c(c6)oc6ccccc67)c5c34)n2)cc1 ; c1ccc(-c2nc(-c3ccc4c(ccc5ccccc54)c3)nc(-c3c(-c4ccc5c(c4)oc4ccccc45)ccc4oc5ccccc5c34)n2)cc1]

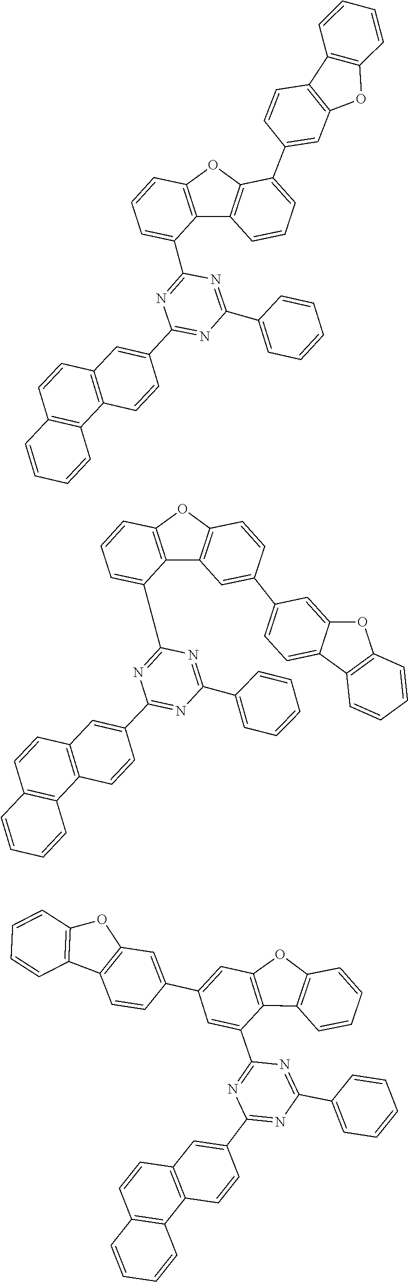 [SMILES: c1ccc(-c2nc(-c3ccc4c(ccc5ccccc54)c3)nc(-c3cccc4oc5c(-c6ccc7c(c6)oc6ccccc67)cccc5c34)n2)cc1 ; c1ccc(-c2nc(-c3ccc4c(ccc5ccccc54)c3)nc(-c3cccc4oc5ccc(-c6ccc7c(c6)oc6ccccc67)cc5c34)n2)cc1 ; c1ccc(-c2nc(-c3ccc4c(ccc5ccccc54)c3)nc(-c3cc(-c4ccc5c(c4)oc4ccccc45)cc4oc5ccccc5c34)n2)cc1]

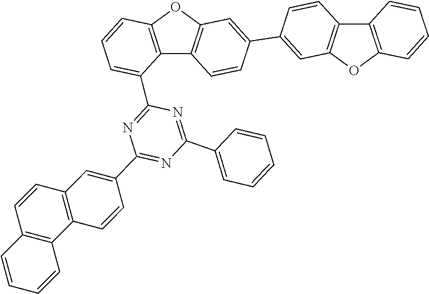 [SMILES: c1ccc(-c2nc(-c3ccc4c(ccc5ccccc54)c3)nc(-c3cccc4oc5cc(-c6ccc7c(c6)oc6ccccc67)ccc5c34)n2)cc1]

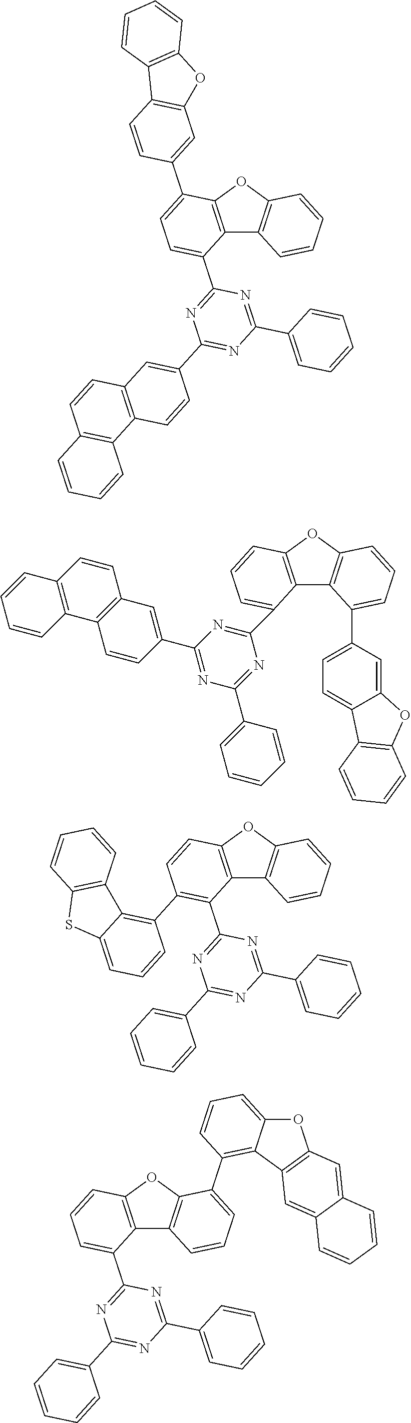

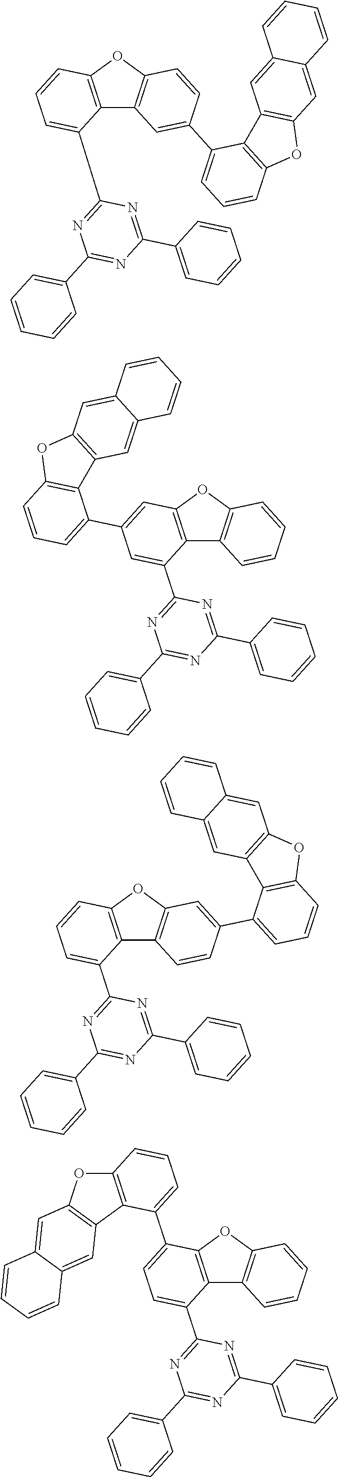

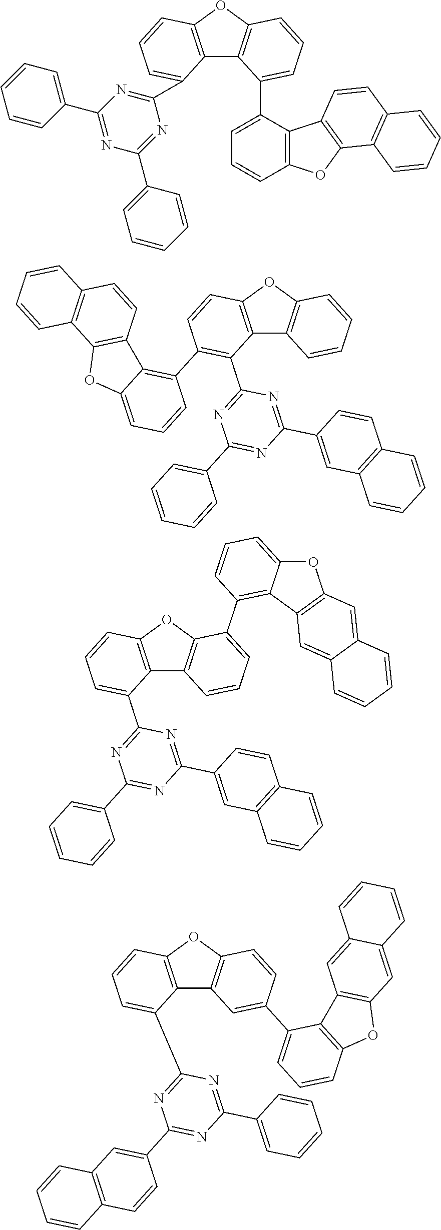

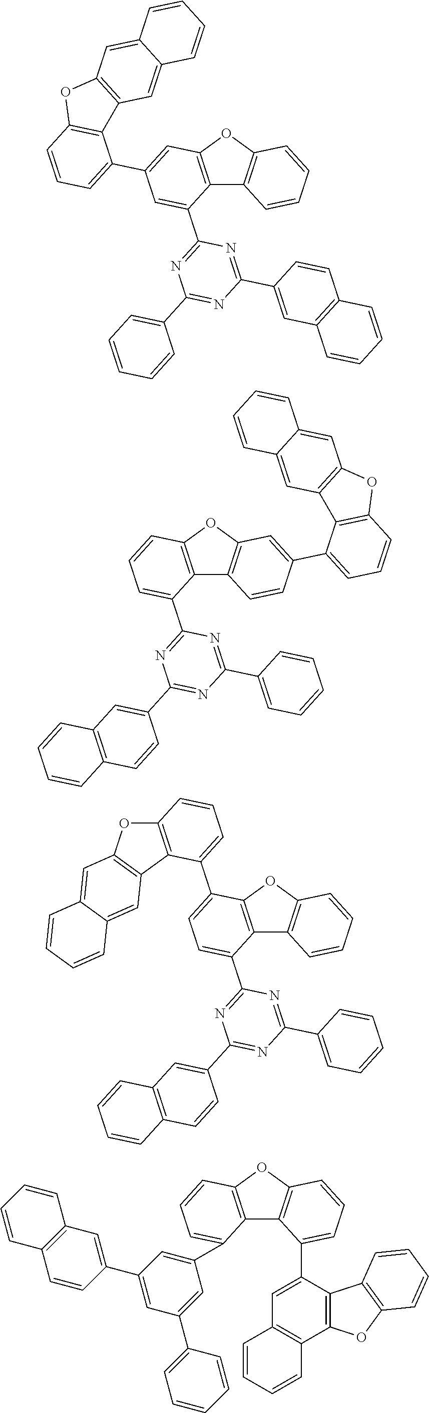 [SMILES: c1ccc(-c2nc(-c3ccc4ccccc4c3)nc(-c3cc(-c4cccc5oc6cc7ccccc7cc6c45)cc4oc5ccccc5c34)n2)cc1 ; c1ccc(-c2nc(-c3ccc4ccccc4c3)nc(-c3cccc4oc5cc(-c6cccc7oc8cc9ccccc9cc8c67)ccc5c34)n2)cc1 ; c1ccc(-c2nc(-c3ccc4ccccc4c3)nc(-c3ccc(-c4cccc5oc6cc7ccccc7cc6c45)c4oc5ccccc5c34)n2)cc1 ; c1ccc(-c2cc(-c3ccc4ccccc4c3)cc(-c3cccc4oc5cccc(-c6cc7ccccc7c7oc8ccccc8c67)c5c34)c2)cc1]

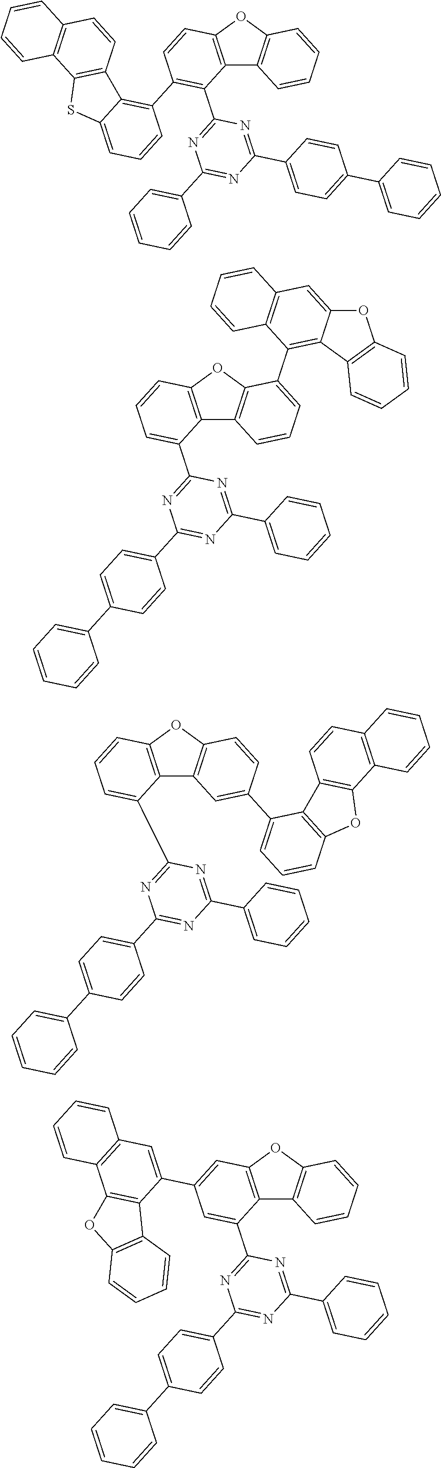

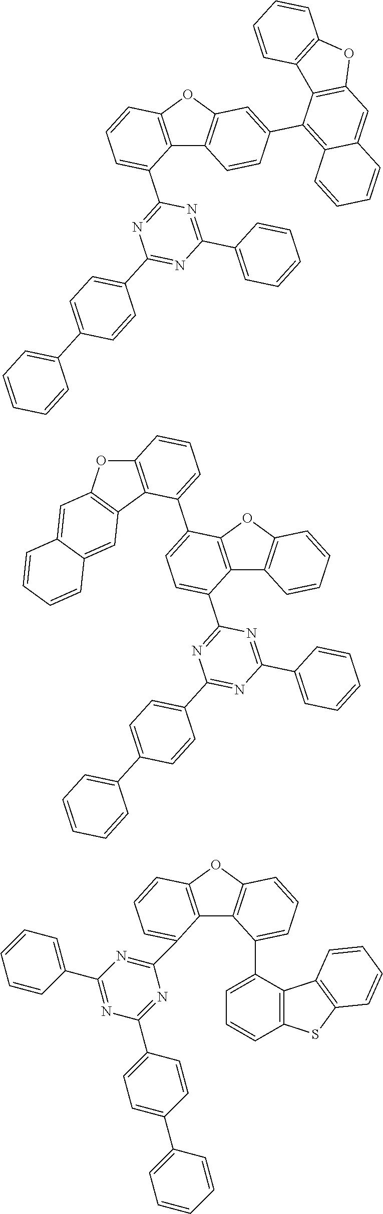 [SMILES: c1ccc(-c2ccc(-c3nc(-c4ccccc4)nc(-c4cccc5oc6cc(-c7c8ccccc8cc8oc9ccccc9c78)ccc6c45)n3)cc2)cc1 ; c1ccc(-c2ccc(-c3nc(-c4ccccc4)nc(-c4ccc(-c5cccc6oc7cc8ccccc8cc7c56)c5oc6ccccc6c45)n3)cc2)cc1 ; c1ccc(-c2ccc(-c3nc(-c4ccccc4)nc(-c4cccc5oc6cccc(-c7cccc8sc9ccccc9c78)c6c45)n3)cc2)cc1]

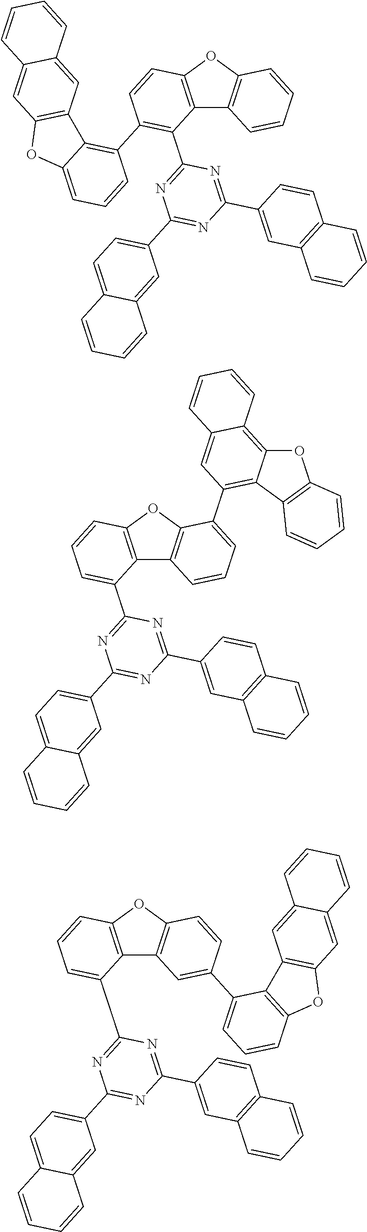 [SMILES: c1ccc2cc(-c3nc(-c4ccc5ccccc5c4)nc(-c4c(-c5cccc6oc7cc8ccccc8cc7c56)ccc5oc6ccccc6c45)n3)ccc2c1 ; c1ccc2cc(-c3nc(-c4ccc5ccccc5c4)nc(-c4cccc5oc6c(-c7cc8ccccc8c8oc9ccccc9c78)cccc6c45)n3)ccc2c1 ; c1ccc2cc(-c3nc(-c4ccc5ccccc5c4)nc(-c4cccc5oc6ccc(-c7cccc8oc9cc%10ccccc%10cc9c78)cc6c45)n3)ccc2c1]

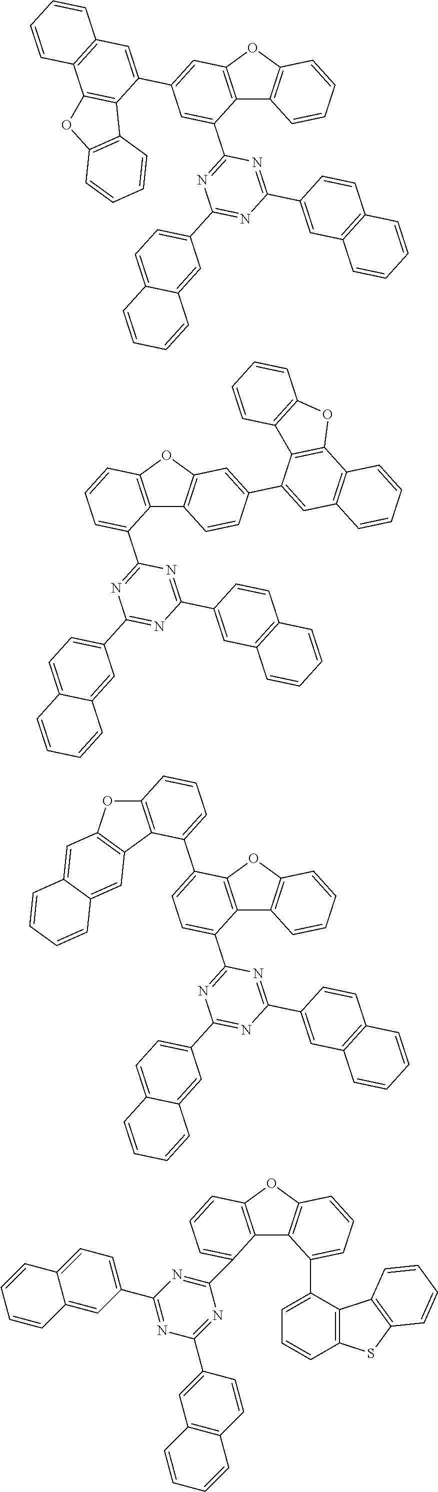 [SMILES: c1ccc2cc(-c3nc(-c4ccc5ccccc5c4)nc(-c4cc(-c5cc6ccccc6c6oc7ccccc7c56)cc5oc6ccccc6c45)n3)ccc2c1 ; c1ccc2cc(-c3nc(-c4ccc5ccccc5c4)nc(-c4cccc5oc6cc(-c7cc8ccccc8c8oc9ccccc9c78)ccc6c45)n3)ccc2c1 ; c1ccc2cc(-c3nc(-c4ccc5ccccc5c4)nc(-c4ccc(-c5cccc6oc7cc8ccccc8cc7c56)c5oc6ccccc6c45)n3)ccc2c1 ; c1ccc2cc(-c3nc(-c4ccc5ccccc5c4)nc(-c4cccc5oc6cccc(-c7cccc8sc9ccccc9c78)c6c45)n3)ccc2c1]

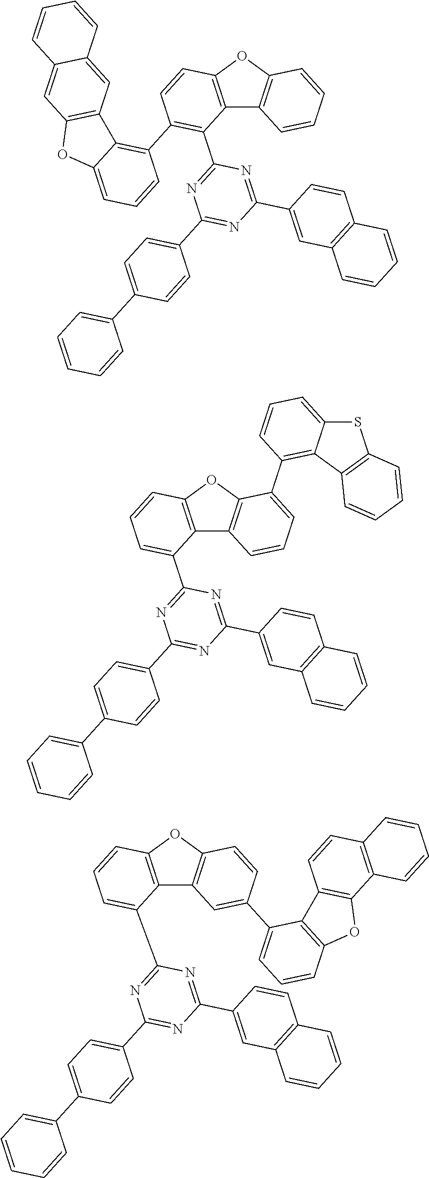 [SMILES: c1ccc(-c2ccc(-c3nc(-c4ccc5ccccc5c4)nc(-c4c(-c5cccc6oc7cc8ccccc8cc7c56)ccc5oc6ccccc6c45)n3)cc2)cc1 ; c1ccc(-c2ccc(-c3nc(-c4ccc5ccccc5c4)nc(-c4cccc5oc6c(-c7cccc8sc9ccccc9c78)cccc6c45)n3)cc2)cc1 ; c1ccc(-c2ccc(-c3nc(-c4ccc5ccccc5c4)nc(-c4cccc5oc6ccc(-c7cccc8oc9c%10ccccc%10ccc9c78)cc6c45)n3)cc2)cc1]

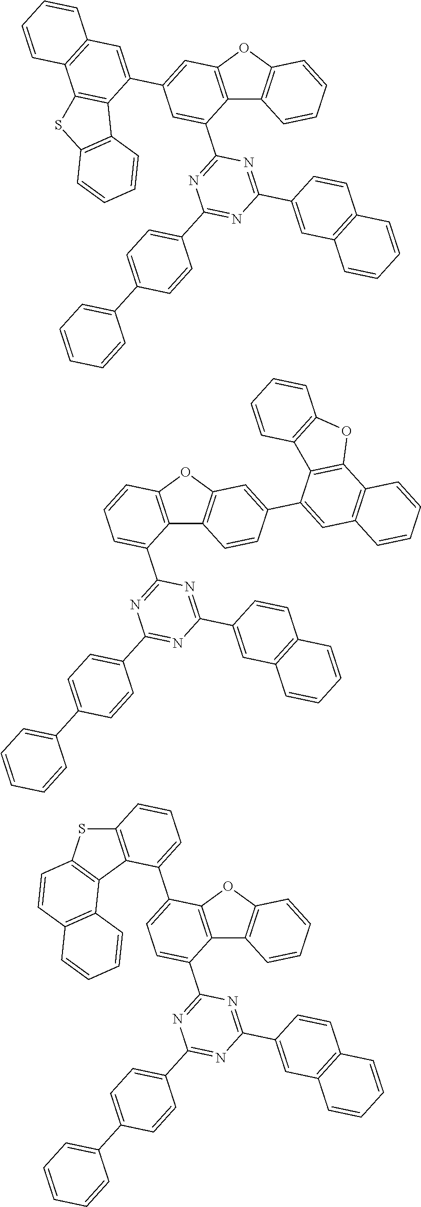 [SMILES: c1ccc(-c2ccc(-c3nc(-c4ccc5ccccc5c4)nc(-c4cc(-c5cc6ccccc6c6sc7ccccc7c56)cc5oc6ccccc6c45)n3)cc2)cc1 ; c1ccc(-c2ccc(-c3nc(-c4ccc5ccccc5c4)nc(-c4cccc5oc6cc(-c7cc8ccccc8c8oc9ccccc9c78)ccc6c45)n3)cc2)cc1 ; c1ccc(-c2ccc(-c3nc(-c4ccc5ccccc5c4)nc(-c4ccc(-c5cccc6sc7ccc8ccccc8c7c56)c5oc6ccccc6c45)n3)cc2)cc1]

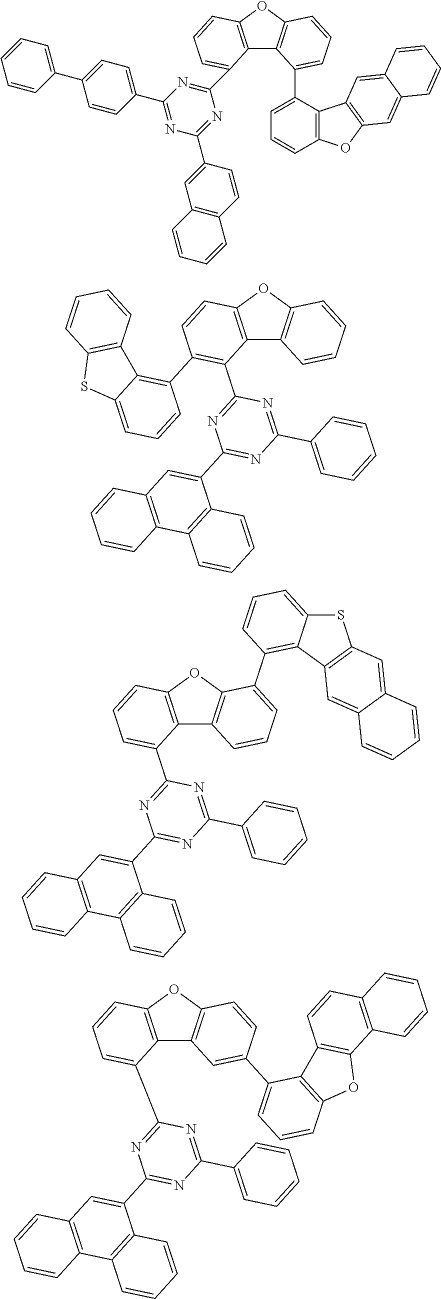

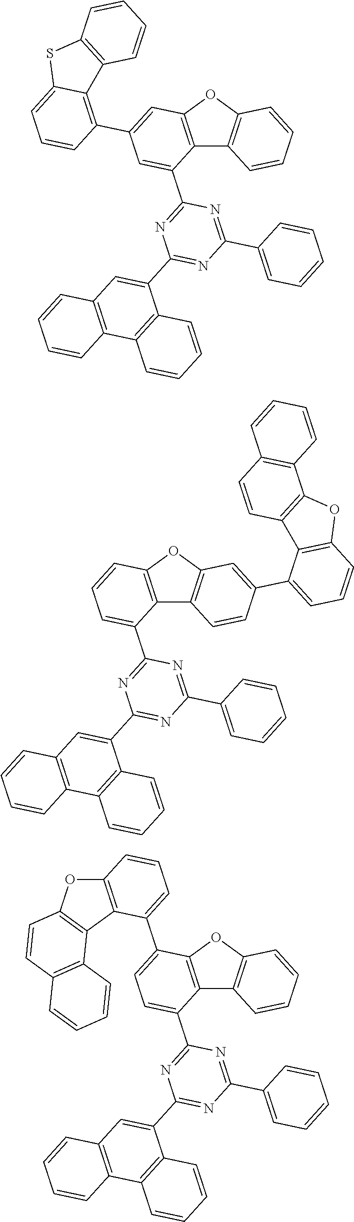

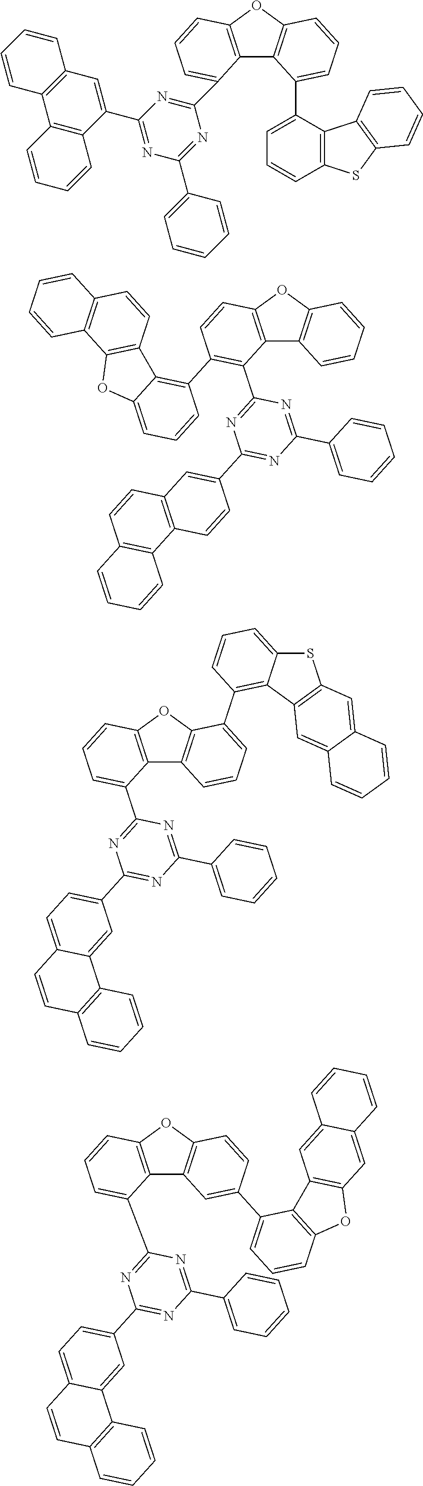 [SMILES: c1ccc(-c2nc(-c3cc4ccccc4c4ccccc34)nc(-c3cccc4oc5cccc(-c6cccc7sc8ccccc8c67)c5c34)n2)cc1 ; c1ccc(-c2nc(-c3ccc4c(ccc5ccccc54)c3)nc(-c3c(-c4cccc5oc6c7ccccc7ccc6c45)ccc4oc5ccccc5c34)n2)cc1 ; c1ccc(-c2nc(-c3ccc4ccc5ccccc5c4c3)nc(-c3cccc4oc5c(-c6cccc7sc8cc9ccccc9cc8c67)cccc5c34)n2)cc1 ; c1ccc(-c2nc(-c3ccc4ccc5ccccc5c4c3)nc(-c3cccc4oc5ccc(-c6cccc7oc8cc9ccccc9cc8c67)cc5c34)n2)cc1]

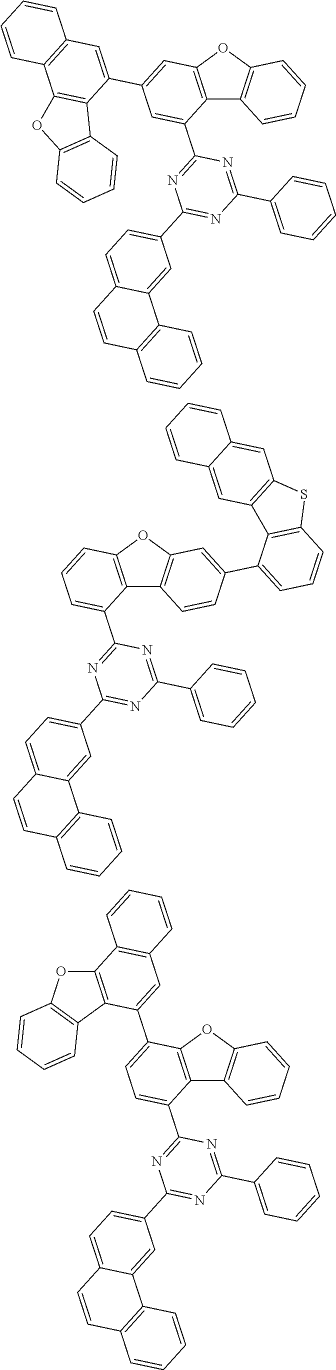

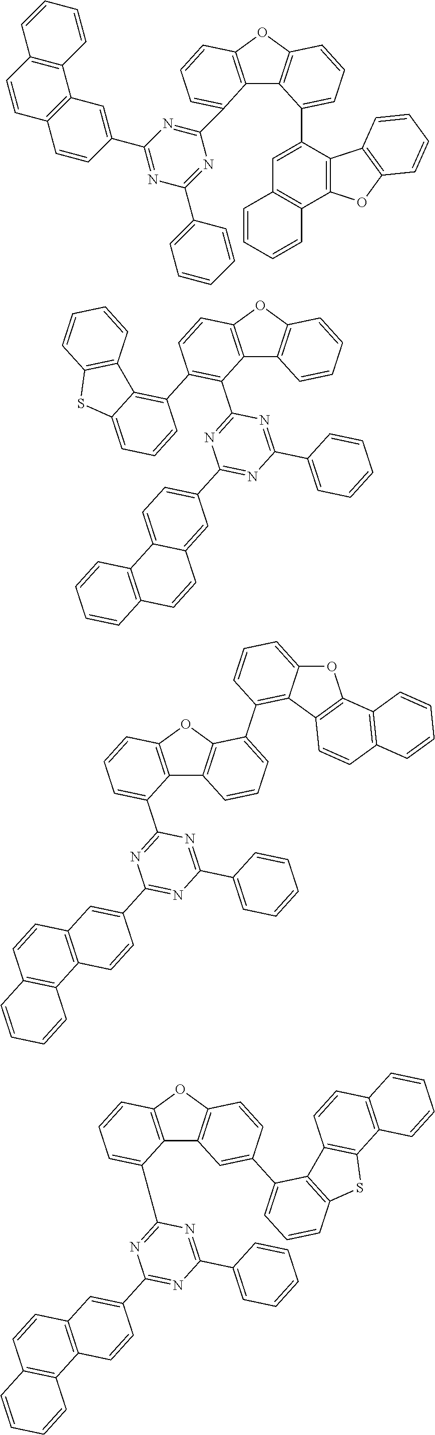

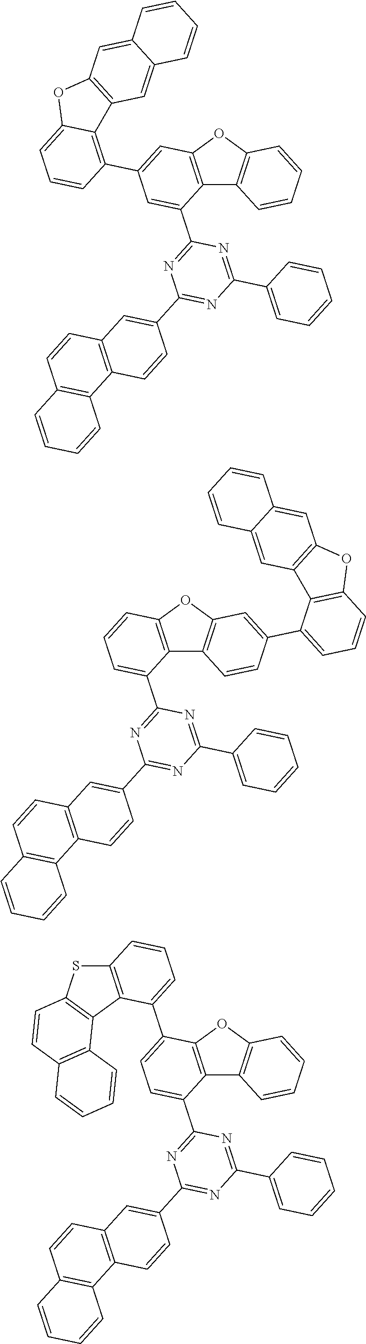 [SMILES: c1ccc(-c2nc(-c3ccc4c(ccc5ccccc54)c3)nc(-c3cc(-c4cccc5oc6cc7ccccc7cc6c45)cc4oc5ccccc5c34)n2)cc1 ; c1ccc(-c2nc(-c3ccc4c(ccc5ccccc54)c3)nc(-c3cccc4oc5cc(-c6cccc7oc8cc9ccccc9cc8c67)ccc5c34)n2)cc1 ; c1ccc(-c2nc(-c3ccc4c(ccc5ccccc54)c3)nc(-c3ccc(-c4cccc5sc6ccc7ccccc7c6c45)c4oc5ccccc5c34)n2)cc1]

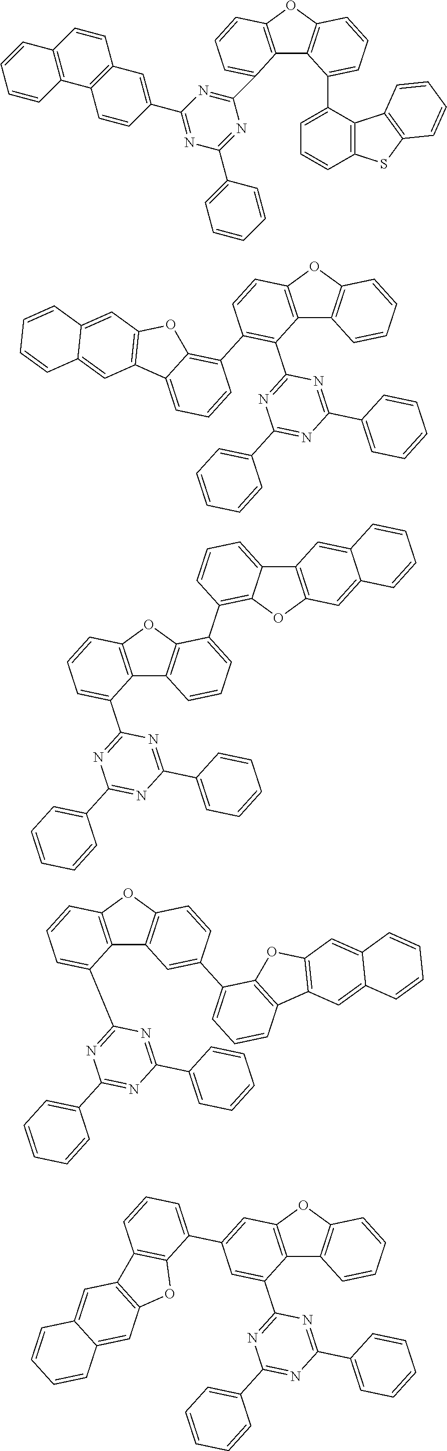

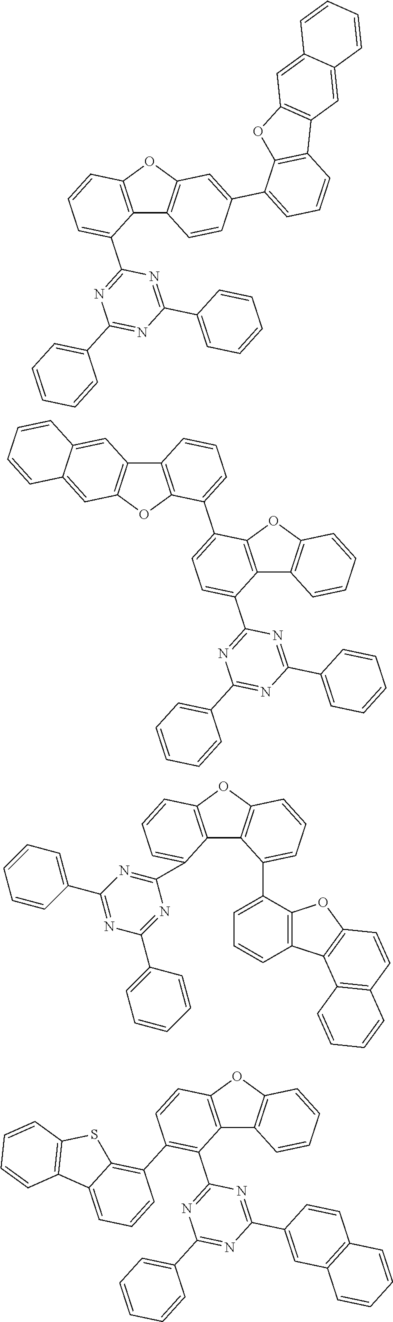

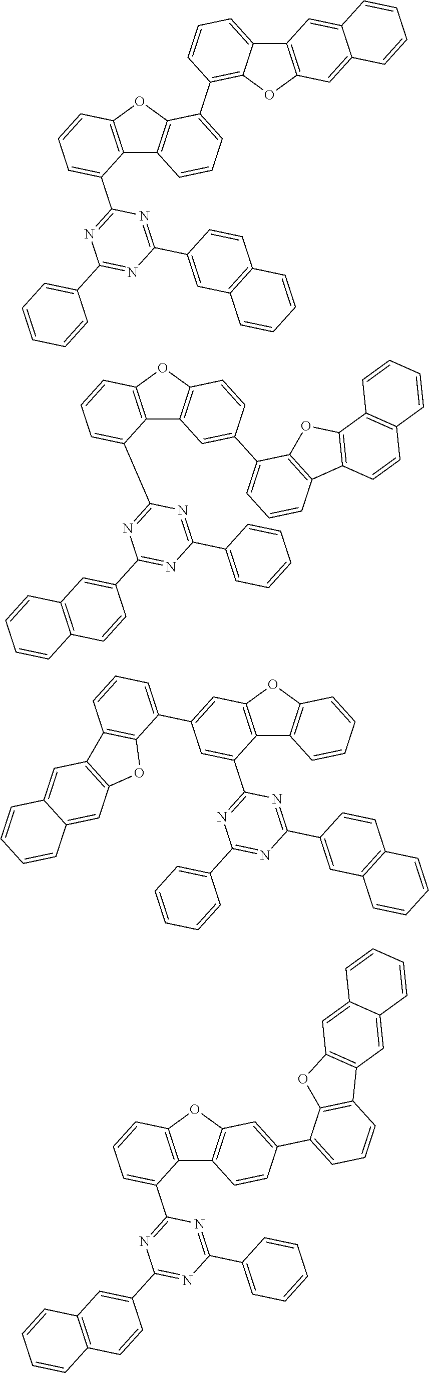

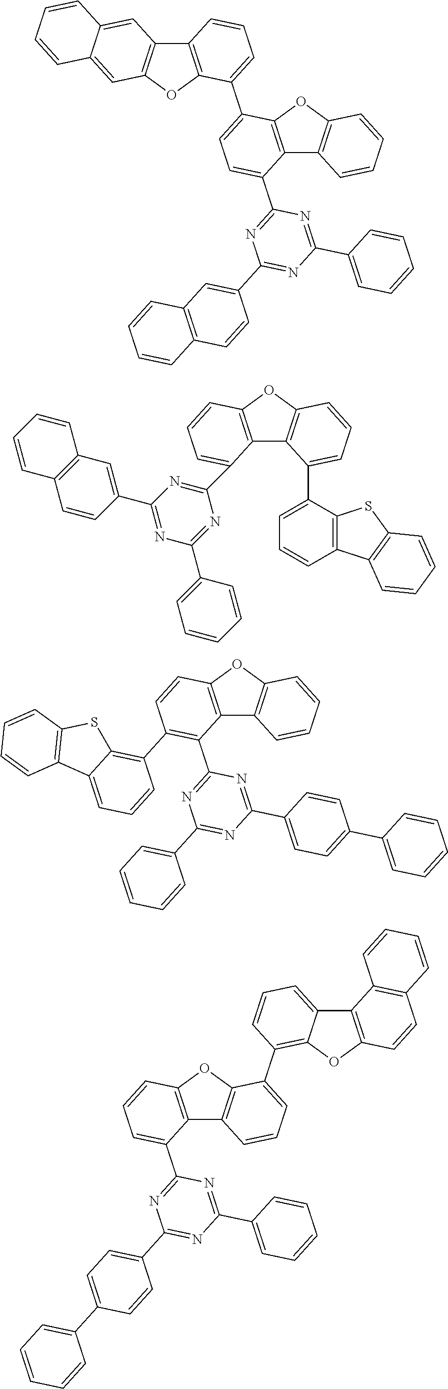

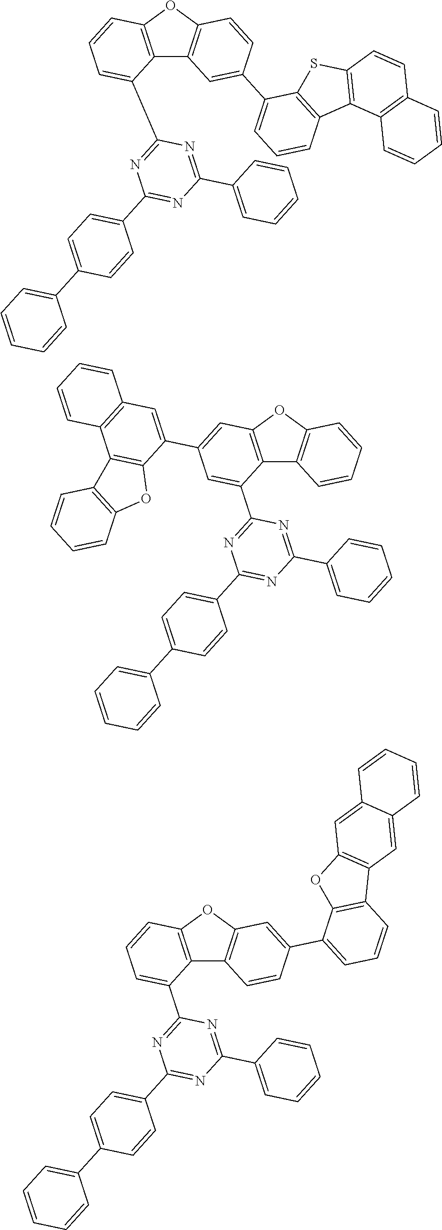

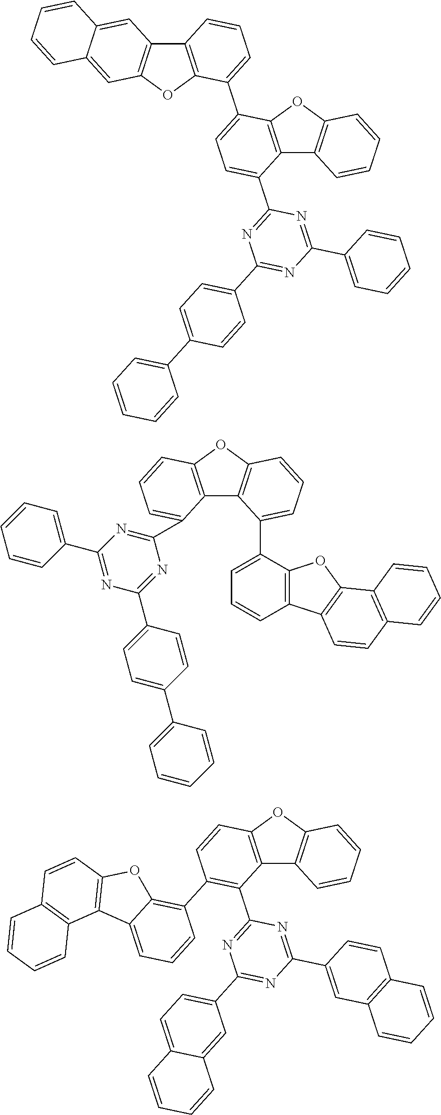

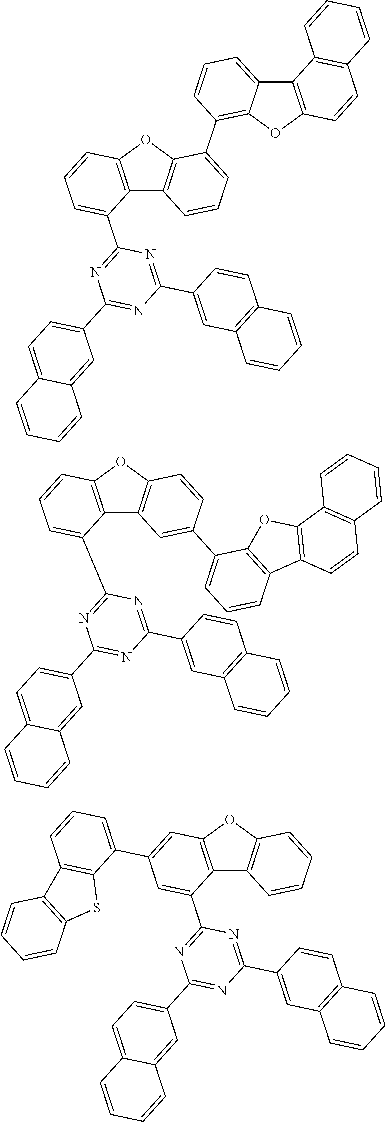

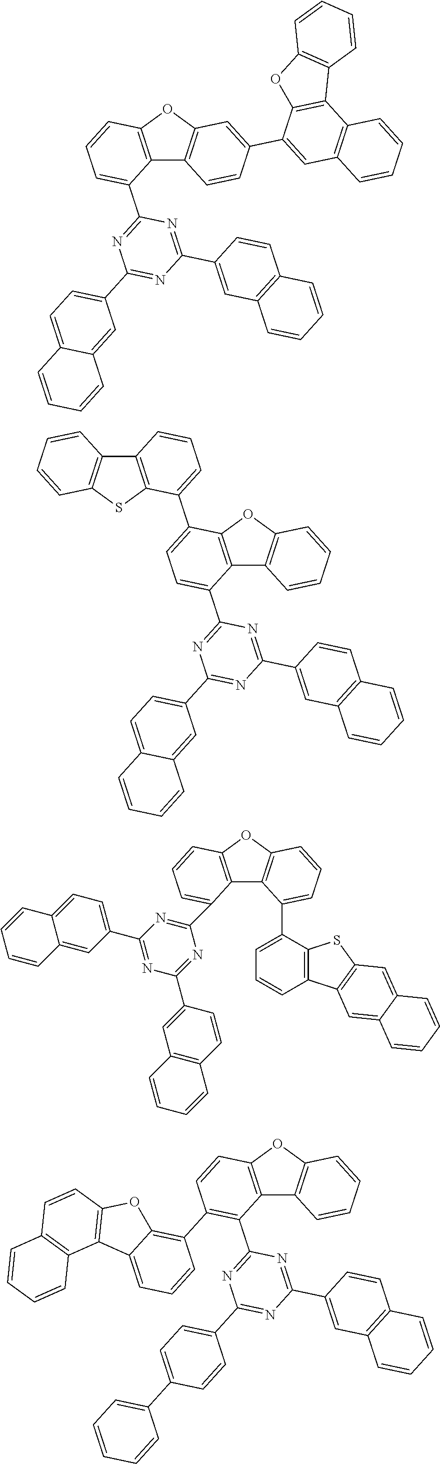

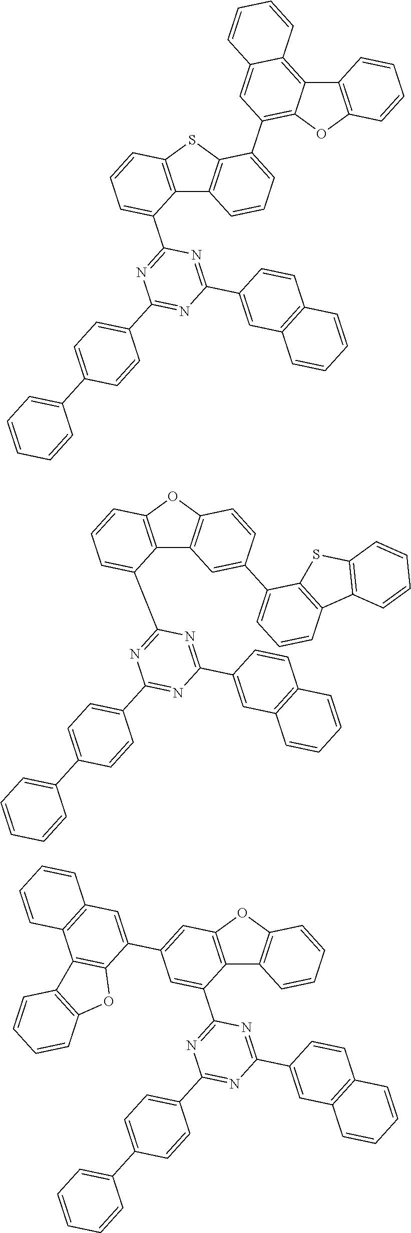

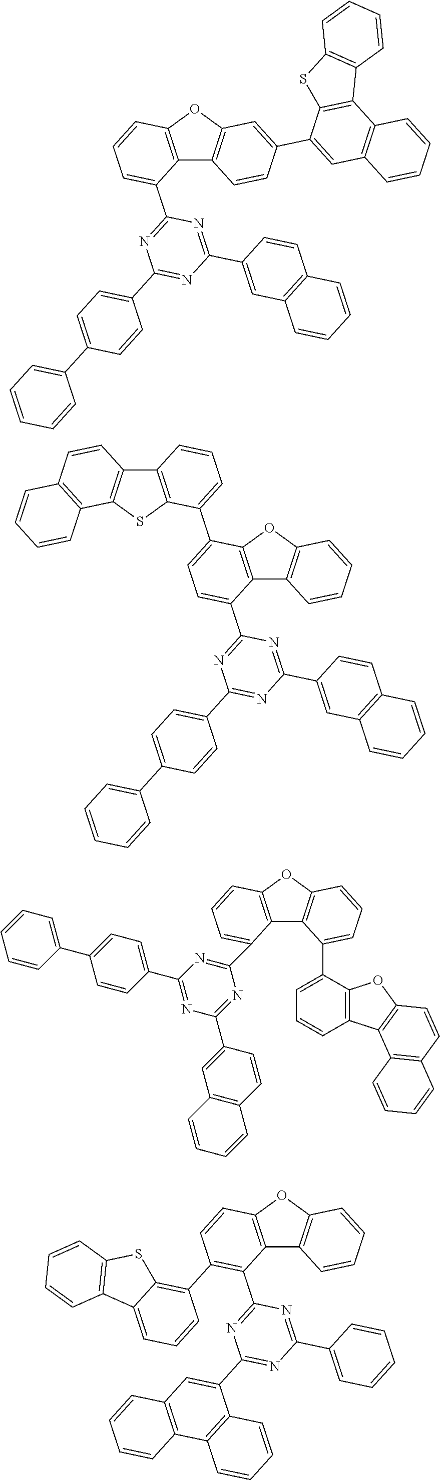

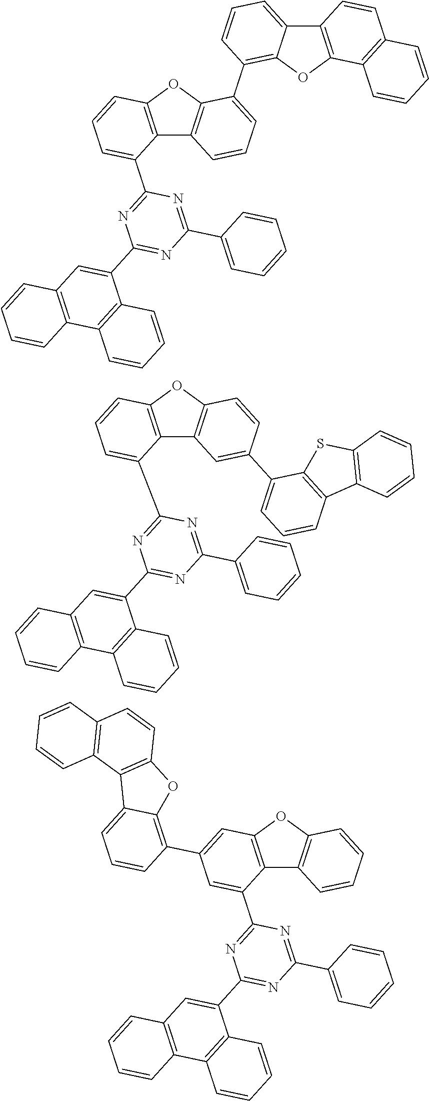

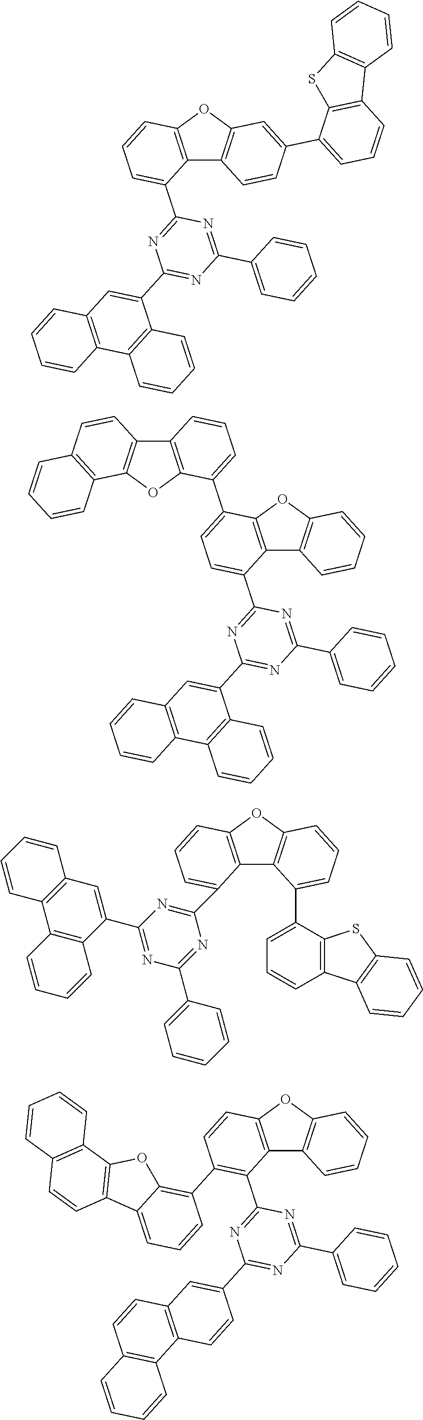

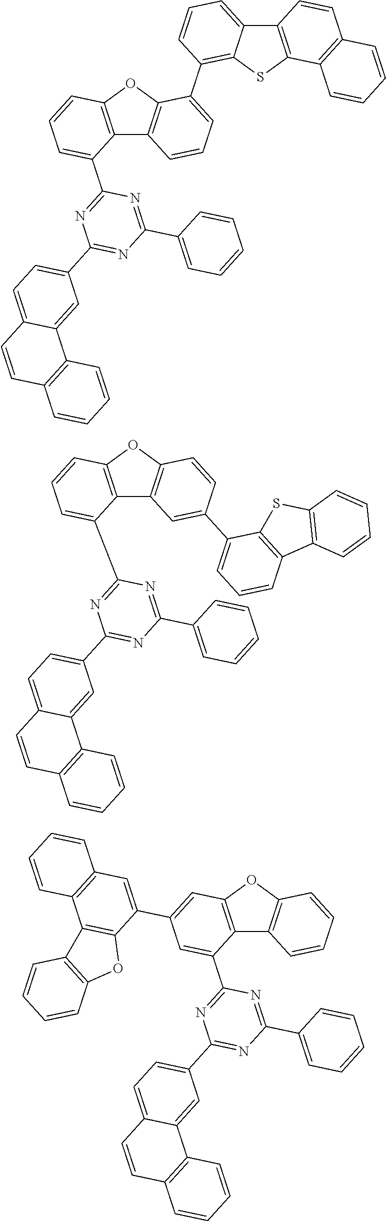

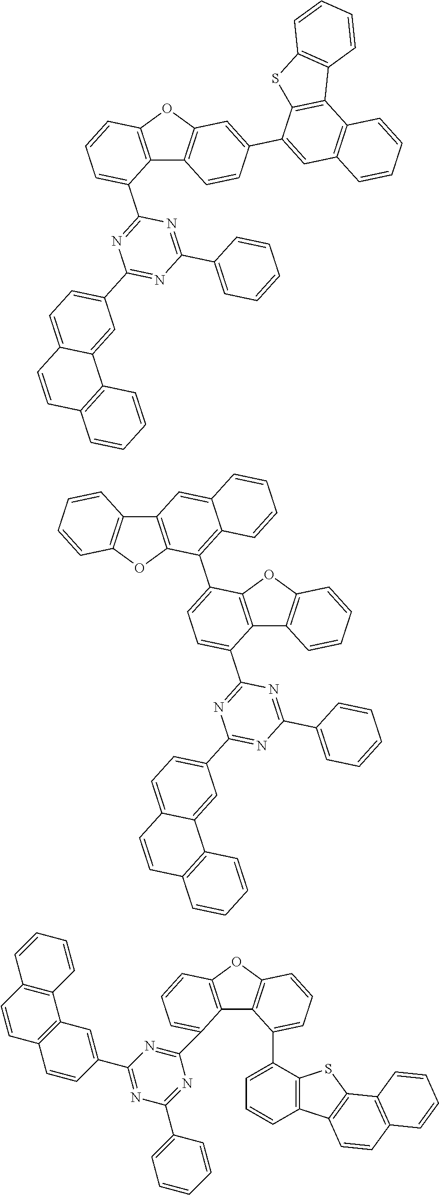 [SMILES: c1ccc(-c2nc(-c3ccc4ccc5ccccc5c4c3)nc(-c3cccc4oc5cc(-c6cc7ccccc7c7c6sc6ccccc67)ccc5c34)n2)cc1 ; c1ccc(-c2nc(-c3ccc4ccc5ccccc5c4c3)nc(-c3ccc(-c4c5ccccc5cc5c4oc4ccccc45)c4oc5ccccc5c34)n2)cc1 ; c1ccc(-c2nc(-c3ccc4ccc5ccccc5c4c3)nc(-c3cccc4oc5cccc(-c6cccc7c6sc6c8ccccc8ccc76)c5c34)n2)cc1]

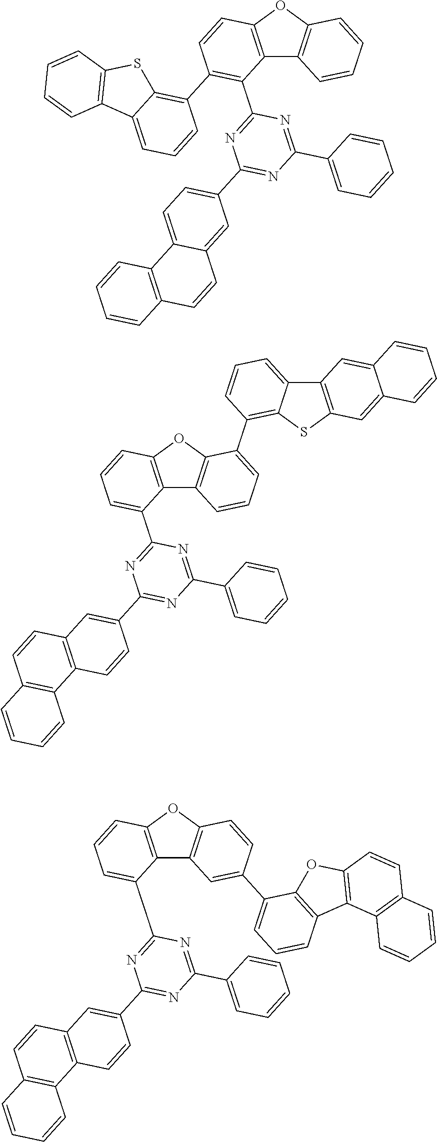 [SMILES: c1ccc(-c2nc(-c3ccc4c(ccc5ccccc54)c3)nc(-c3c(-c4cccc5c4sc4ccccc45)ccc4oc5ccccc5c34)n2)cc1 ; c1ccc(-c2nc(-c3ccc4c(ccc5ccccc54)c3)nc(-c3cccc4oc5c(-c6cccc7c6sc6cc8ccccc8cc67)cccc5c34)n2)cc1 ; c1ccc(-c2nc(-c3ccc4c(ccc5ccccc54)c3)nc(-c3cccc4oc5ccc(-c6cccc7c6oc6ccc8ccccc8c67)cc5c34)n2)cc1]

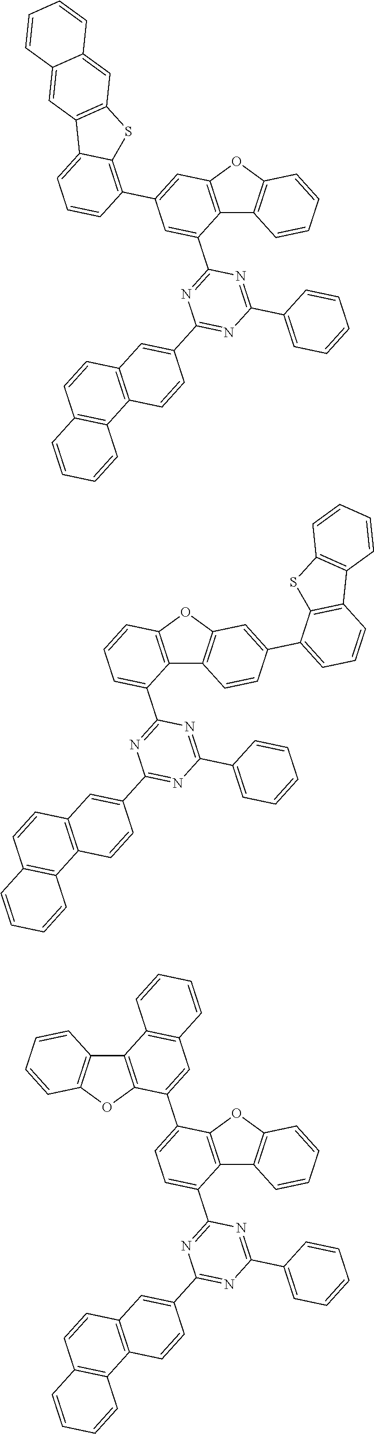 [SMILES: c1ccc(-c2nc(-c3ccc4c(ccc5ccccc54)c3)nc(-c3cc(-c4cccc5c4sc4cc6ccccc6cc45)cc4oc5ccccc5c34)n2)cc1 ; c1ccc(-c2nc(-c3ccc4c(ccc5ccccc54)c3)nc(-c3cccc4oc5cc(-c6cccc7c6sc6ccccc67)ccc5c34)n2)cc1 ; c1ccc(-c2nc(-c3ccc4c(ccc5ccccc54)c3)nc(-c3ccc(-c4cc5ccccc5c5c4oc4ccccc45)c4oc5ccccc5c34)n2)cc1]

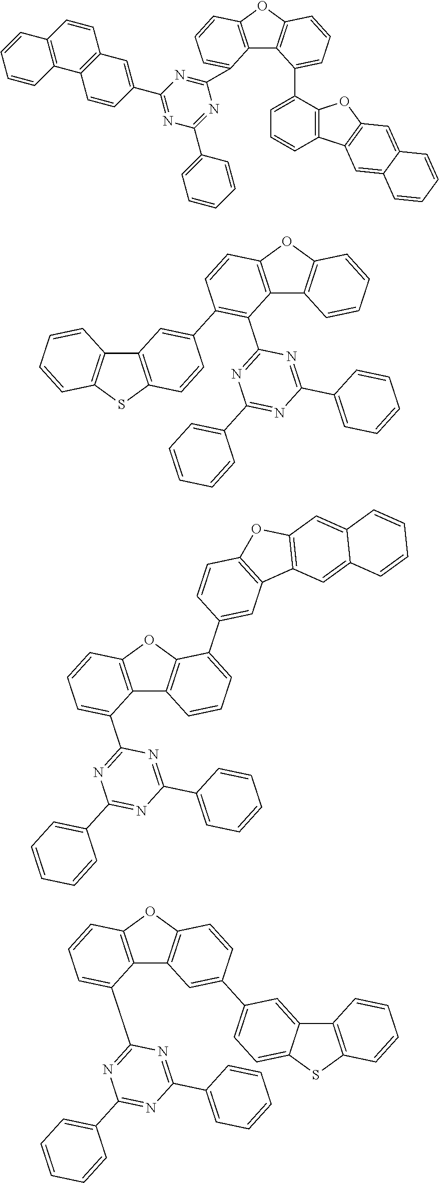

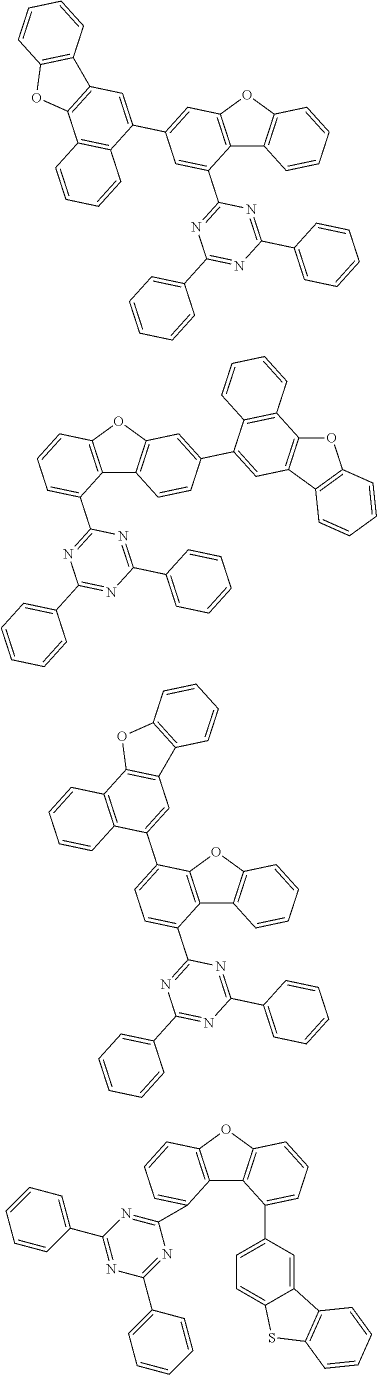 [SMILES: c1ccc(-c2nc(-c3ccccc3)nc(-c3cc(-c4cc5c6ccccc6oc5c5ccccc45)cc4oc5ccccc5c34)n2)cc1 ; c1ccc(-c2nc(-c3ccccc3)nc(-c3cccc4oc5cc(-c6cc7c8ccccc8oc7c7ccccc67)ccc5c34)n2)cc1 ; c1ccc(-c2nc(-c3ccccc3)nc(-c3ccc(-c4cc5c6ccccc6oc5c5ccccc45)c4oc5ccccc5c34)n2)cc1 ; c1ccc(-c2nc(-c3ccccc3)nc(-c3cccc4oc5cccc(-c6ccc7sc8ccccc8c7c6)c5c34)n2)cc1]

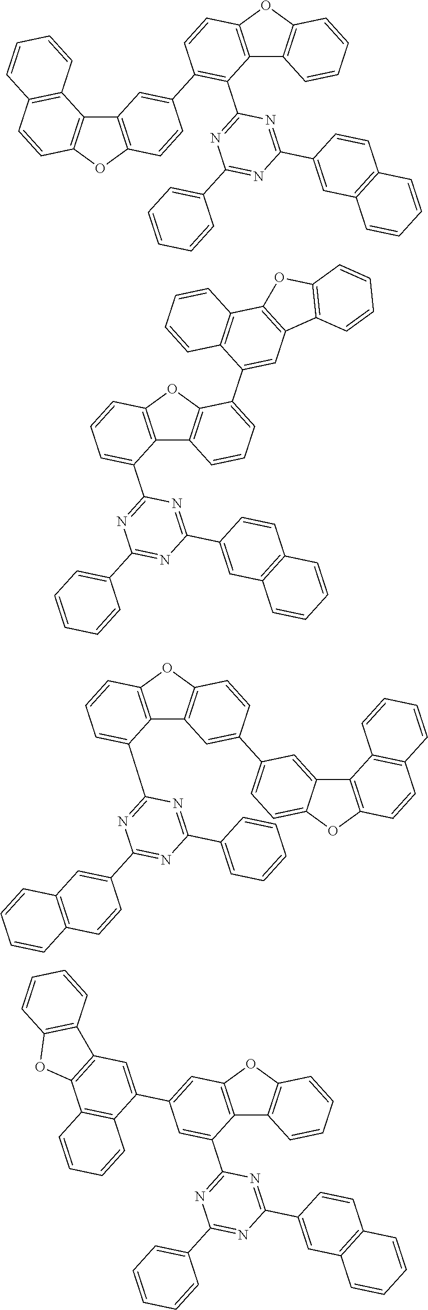

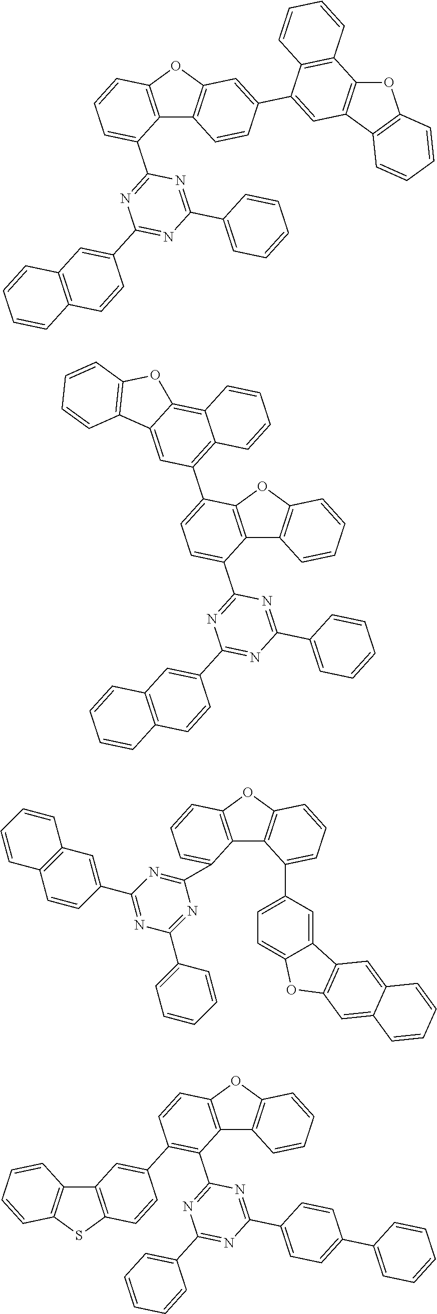 [SMILES: c1ccc(-c2nc(-c3ccc4ccccc4c3)nc(-c3cccc4oc5cc(-c6cc7c8ccccc8oc7c7ccccc67)ccc5c34)n2)cc1 ; c1ccc(-c2nc(-c3ccc4ccccc4c3)nc(-c3ccc(-c4cc5c6ccccc6oc5c5ccccc45)c4oc5ccccc5c34)n2)cc1 ; c1ccc(-c2nc(-c3ccc4ccccc4c3)nc(-c3cccc4oc5cccc(-c6ccc7oc8cc9ccccc9cc8c7c6)c5c34)n2)cc1 ; c1ccc(-c2ccc(-c3nc(-c4ccccc4)nc(-c4c(-c5ccc6sc7ccccc7c6c5)ccc5oc6ccccc6c45)n3)cc2)cc1]

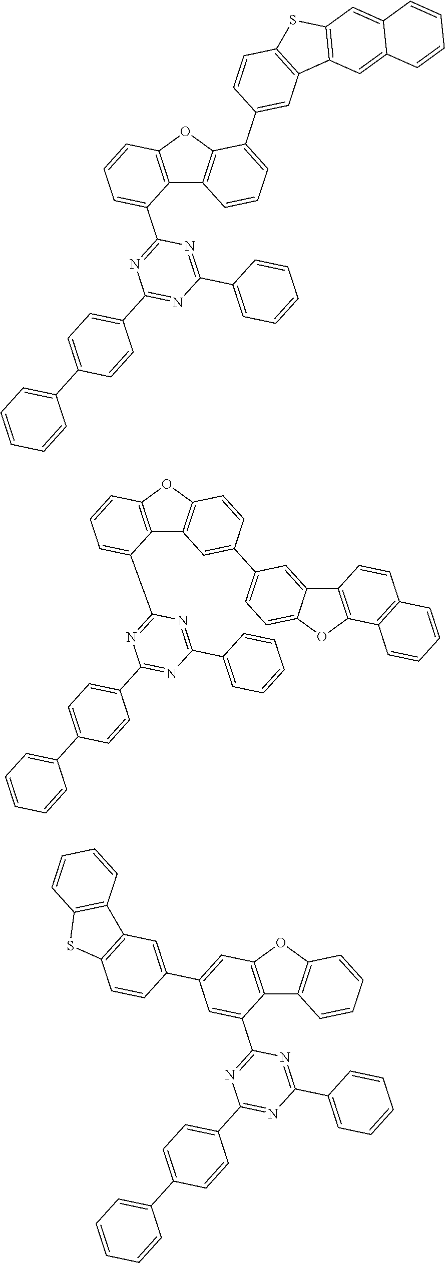

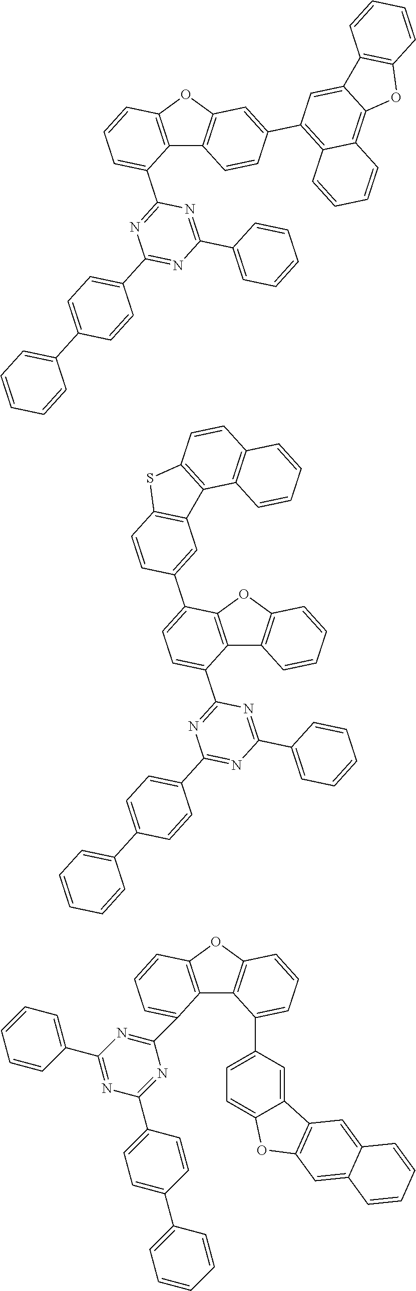 [SMILES: c1ccc(-c2ccc(-c3nc(-c4ccccc4)nc(-c4cccc5oc6cc(-c7cc8c9ccccc9oc8c8ccccc78)ccc6c45)n3)cc2)cc1 ; c1ccc(-c2ccc(-c3nc(-c4ccccc4)nc(-c4ccc(-c5ccc6sc7ccc8ccccc8c7c6c5)c5oc6ccccc6c45)n3)cc2)cc1 ; c1ccc(-c2ccc(-c3nc(-c4ccccc4)nc(-c4cccc5oc6cccc(-c7ccc8oc9cc%10ccccc%10cc9c8c7)c6c45)n3)cc2)cc1]

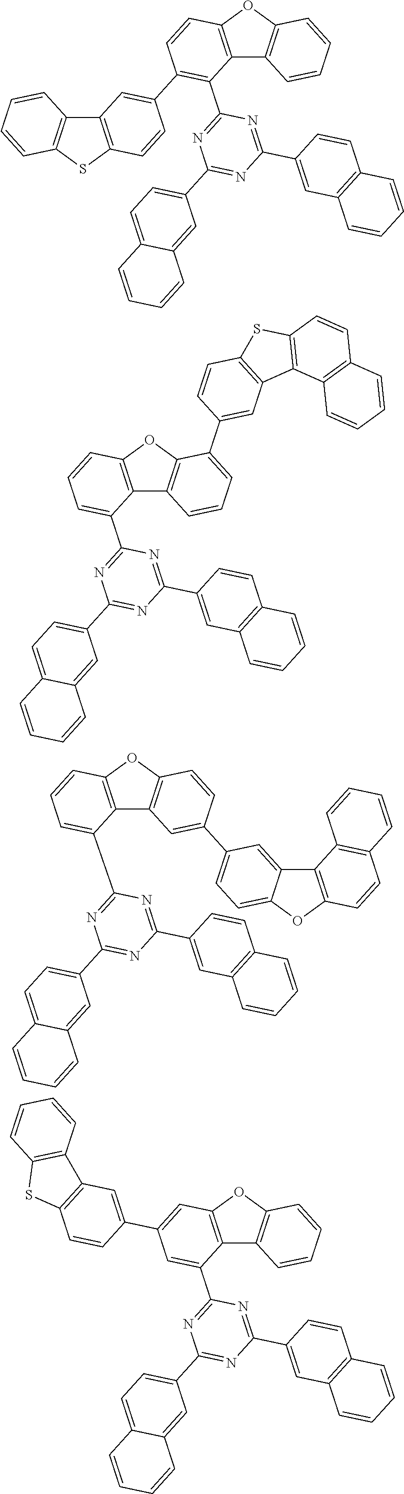

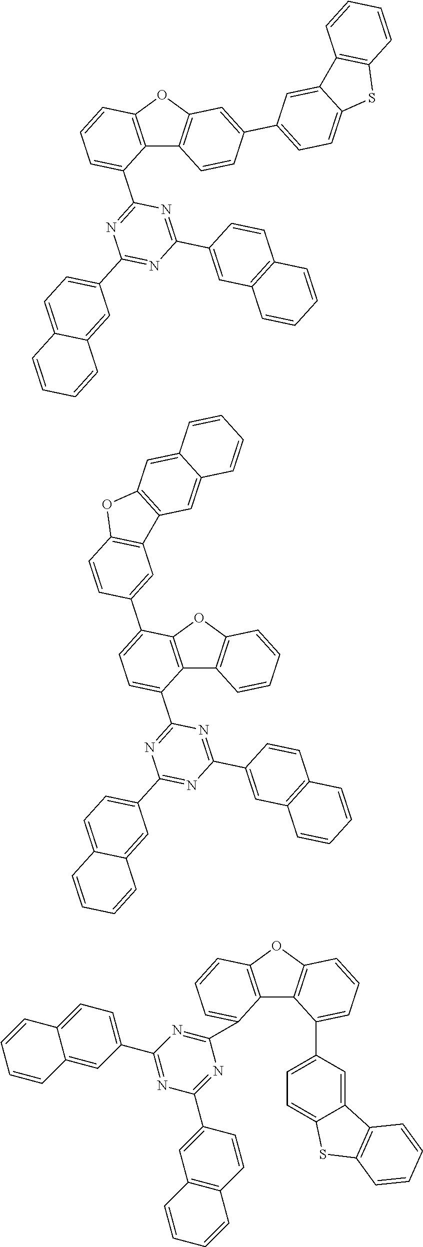 [SMILES: c1ccc2cc(-c3nc(-c4ccc5ccccc5c4)nc(-c4cccc5oc6cc(-c7ccc8sc9ccccc9c8c7)ccc6c45)n3)ccc2c1 ; c1ccc2cc(-c3nc(-c4ccc5ccccc5c4)nc(-c4ccc(-c5ccc6oc7cc8ccccc8cc7c6c5)c5oc6ccccc6c45)n3)ccc2c1 ; c1ccc2cc(-c3nc(-c4ccc5ccccc5c4)nc(-c4cccc5oc6cccc(-c7ccc8sc9ccccc9c8c7)c6c45)n3)ccc2c1]

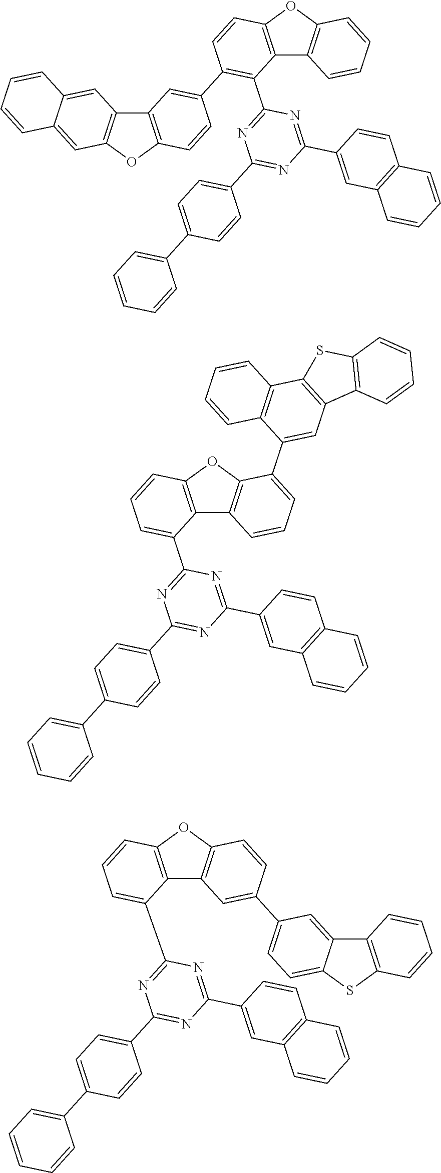 [SMILES: c1ccc(-c2ccc(-c3nc(-c4ccc5ccccc5c4)nc(-c4c(-c5ccc6oc7cc8ccccc8cc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8c9ccccc9sc8c8ccccc78)cccc6c45)n3)cc2)cc1 ; c1ccc(-c2ccc(-c3nc(-c4ccc5ccccc5c4)nc(-c4cccc5oc6ccc(-c7ccc8sc9ccccc9c8c7)cc6c45)n3)cc2)cc1]

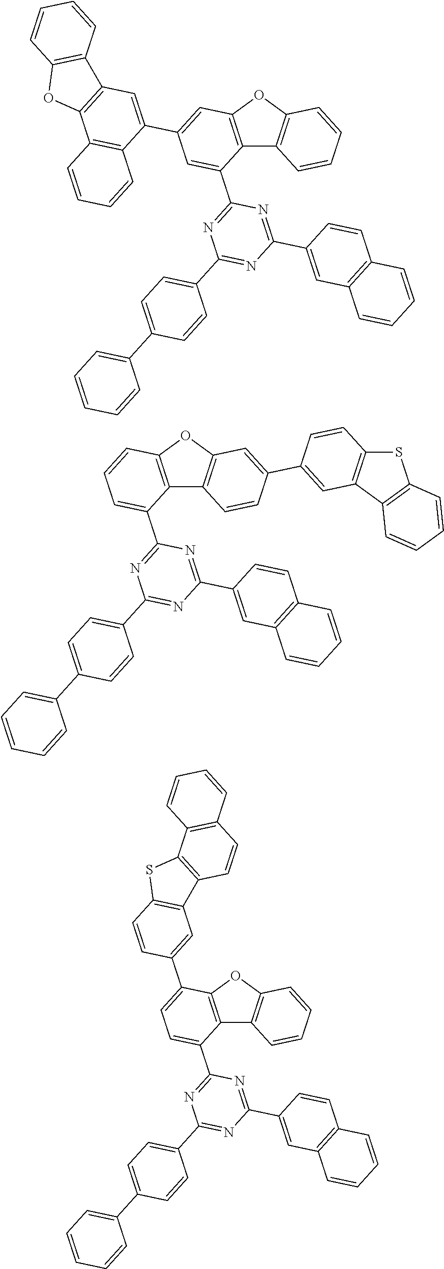

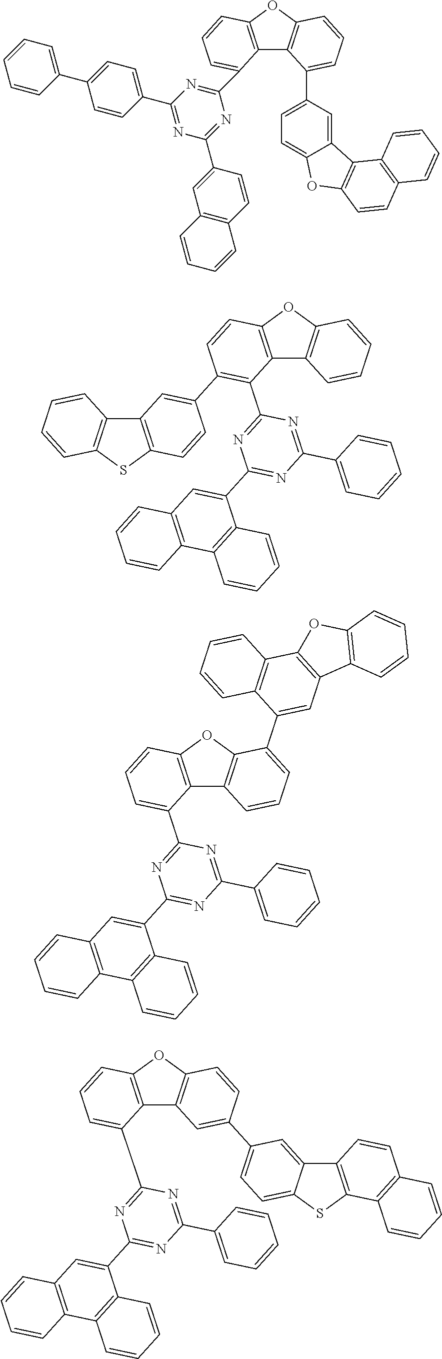

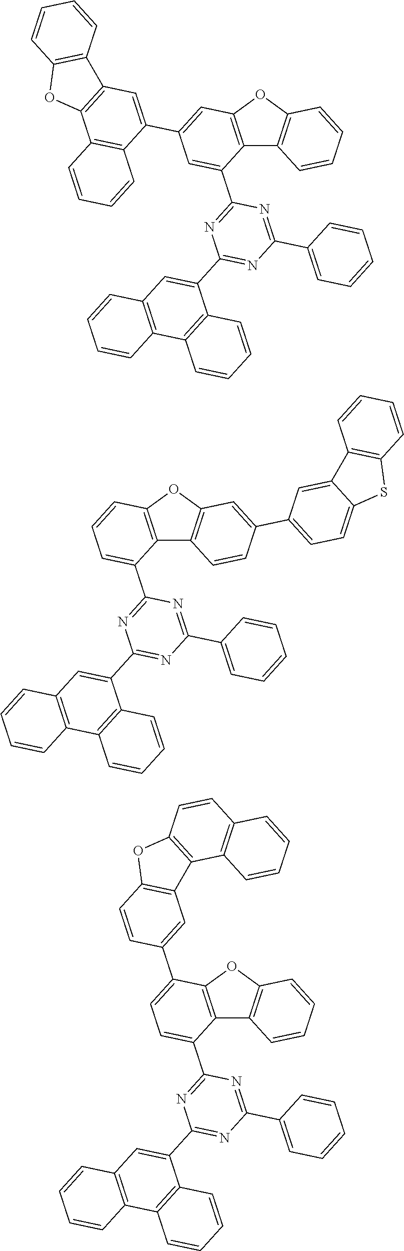 [SMILES: c1ccc(-c2nc(-c3cc4ccccc4c4ccccc34)nc(-c3cc(-c4cc5c6ccccc6oc5c5ccccc45)cc4oc5ccccc5c34)n2)cc1 ; c1ccc(-c2nc(-c3cc4ccccc4c4ccccc34)nc(-c3cccc4oc5cc(-c6ccc7sc8ccccc8c7c6)ccc5c34)n2)cc1 ; c1ccc(-c2nc(-c3cc4ccccc4c4ccccc34)nc(-c3ccc(-c4ccc5oc6ccc7ccccc7c6c5c4)c4oc5ccccc5c34)n2)cc1]

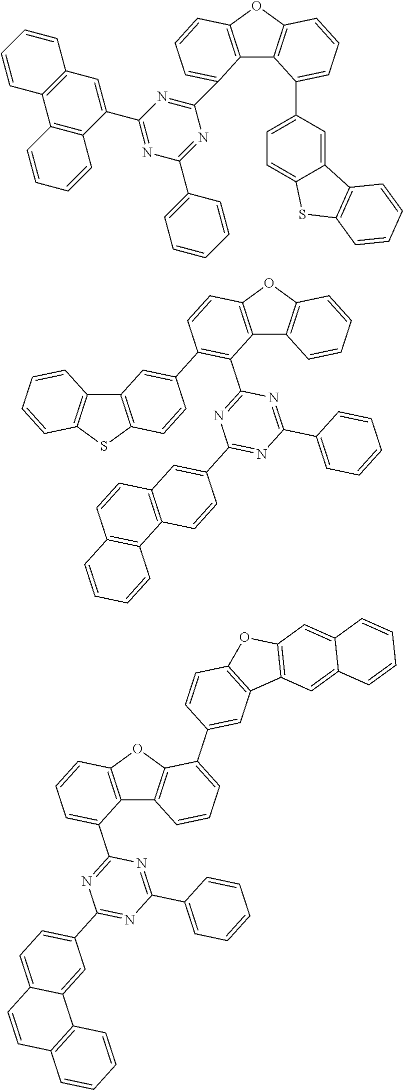

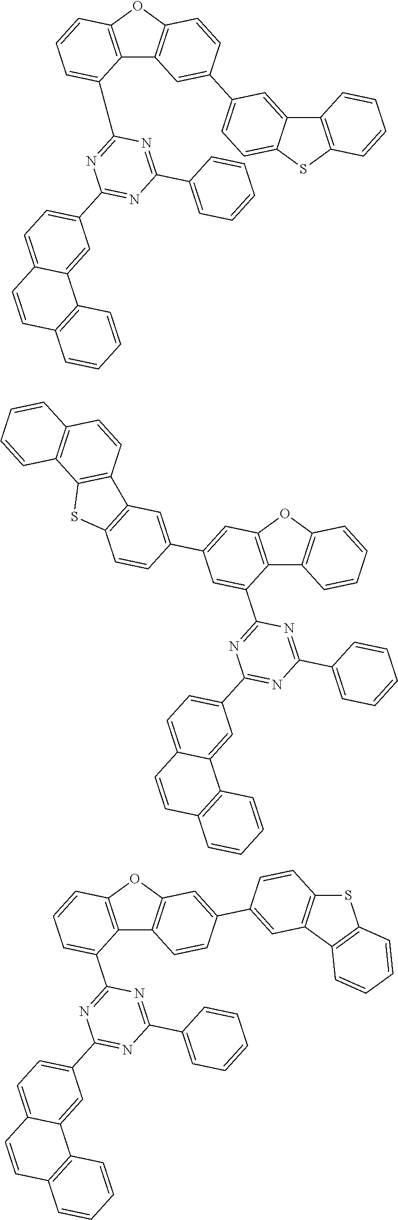

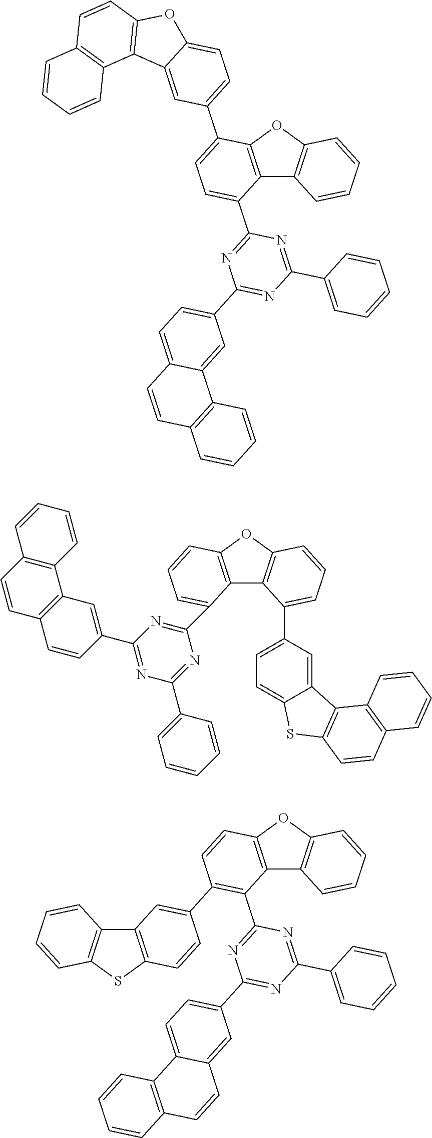 [SMILES: c1ccc(-c2nc(-c3ccc4ccc5ccccc5c4c3)nc(-c3ccc(-c4ccc5oc6ccc7ccccc7c6c5c4)c4oc5ccccc5c34)n2)cc1 ; c1ccc(-c2nc(-c3ccc4ccc5ccccc5c4c3)nc(-c3cccc4oc5cccc(-c6ccc7sc8ccc9ccccc9c8c7c6)c5c34)n2)cc1 ; c1ccc(-c2nc(-c3ccc4c(ccc5ccccc54)c3)nc(-c3c(-c4ccc5sc6ccccc6c5c4)ccc4oc5ccccc5c34)n2)cc1]

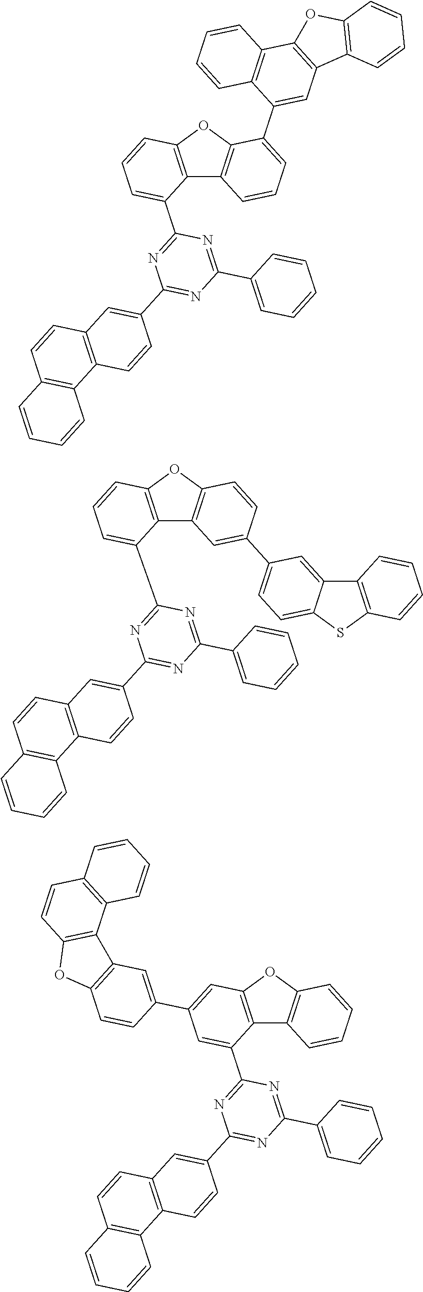 [SMILES: c1ccc(-c2nc(-c3ccc4c(ccc5ccccc54)c3)nc(-c3cccc4oc5c(-c6cc7c8ccccc8oc7c7ccccc67)cccc5c34)n2)cc1 ; c1ccc(-c2nc(-c3ccc4c(ccc5ccccc54)c3)nc(-c3cccc4oc5ccc(-c6ccc7sc8ccccc8c7c6)cc5c34)n2)cc1 ; c1ccc(-c2nc(-c3ccc4c(ccc5ccccc54)c3)nc(-c3cc(-c4ccc5oc6ccc7ccccc7c6c5c4)cc4oc5ccccc5c34)n2)cc1]

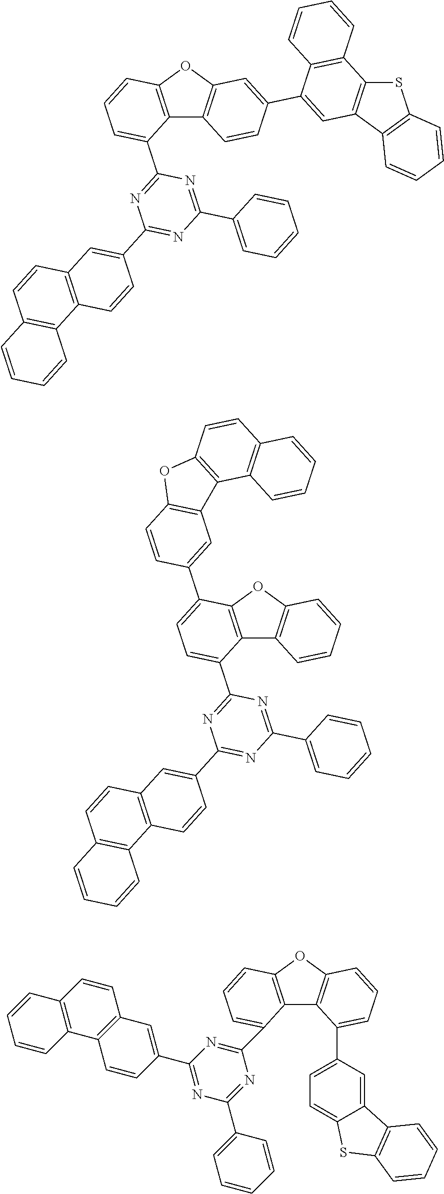 [SMILES: c1ccc(-c2nc(-c3ccc4c(ccc5ccccc54)c3)nc(-c3cccc4oc5cc(-c6cc7c8ccccc8sc7c7ccccc67)ccc5c34)n2)cc1 ; c1ccc(-c2nc(-c3ccc4c(ccc5ccccc54)c3)nc(-c3ccc(-c4ccc5oc6ccc7ccccc7c6c5c4)c4oc5ccccc5c34)n2)cc1 ; c1ccc(-c2nc(-c3ccc4c(ccc5ccccc54)c3)nc(-c3cccc4oc5cccc(-c6ccc7sc8ccccc8c7c6)c5c34)n2)cc1]

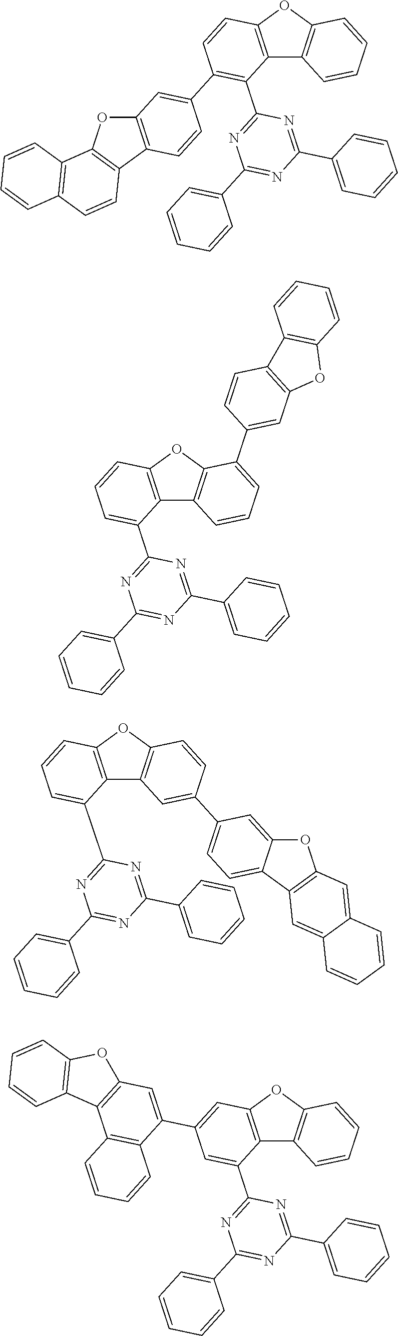

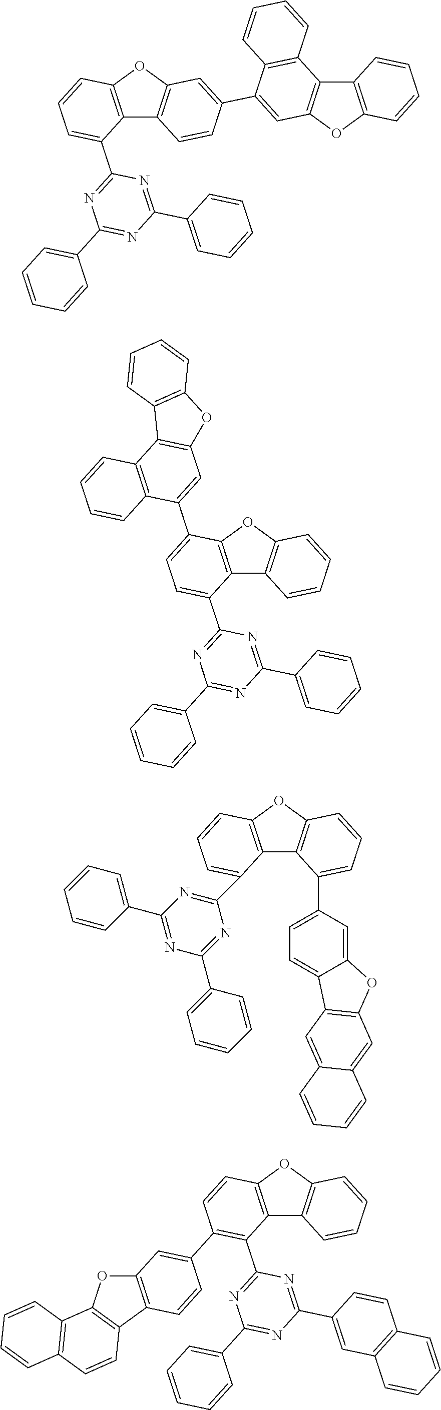 [SMILES: c1ccc(-c2nc(-c3ccccc3)nc(-c3cccc4oc5cc(-c6cc7oc8ccccc8c7c7ccccc67)ccc5c34)n2)cc1 ; c1ccc(-c2nc(-c3ccccc3)nc(-c3ccc(-c4cc5oc6ccccc6c5c5ccccc45)c4oc5ccccc5c34)n2)cc1 ; c1ccc(-c2nc(-c3ccccc3)nc(-c3cccc4oc5cccc(-c6ccc7c(c6)oc6cc8ccccc8cc67)c5c34)n2)cc1 ; c1ccc(-c2nc(-c3ccc4ccccc4c3)nc(-c3c(-c4ccc5c(c4)oc4c6ccccc6ccc54)ccc4oc5ccccc5c34)n2)cc1]

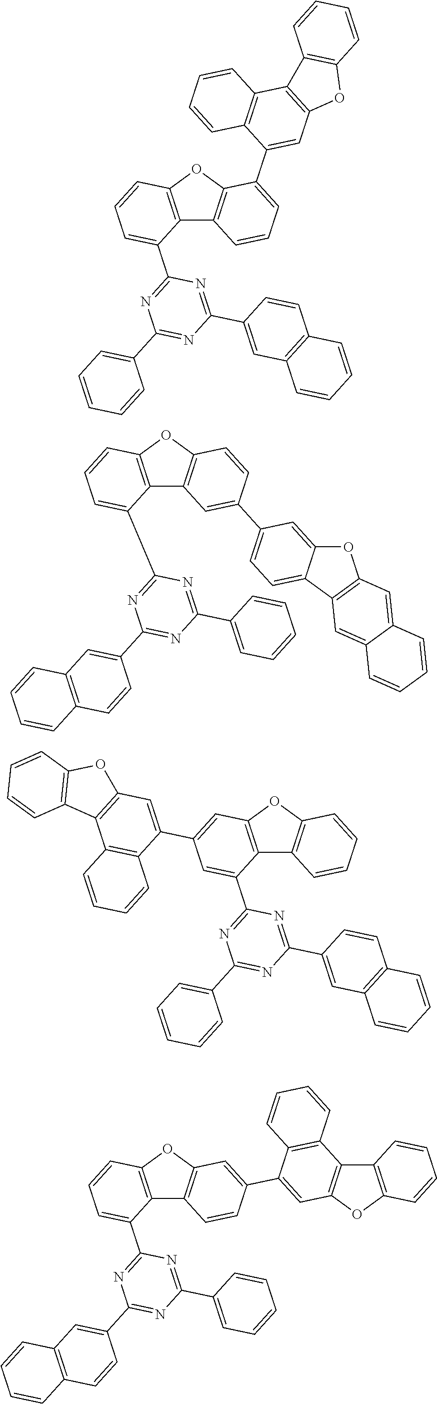

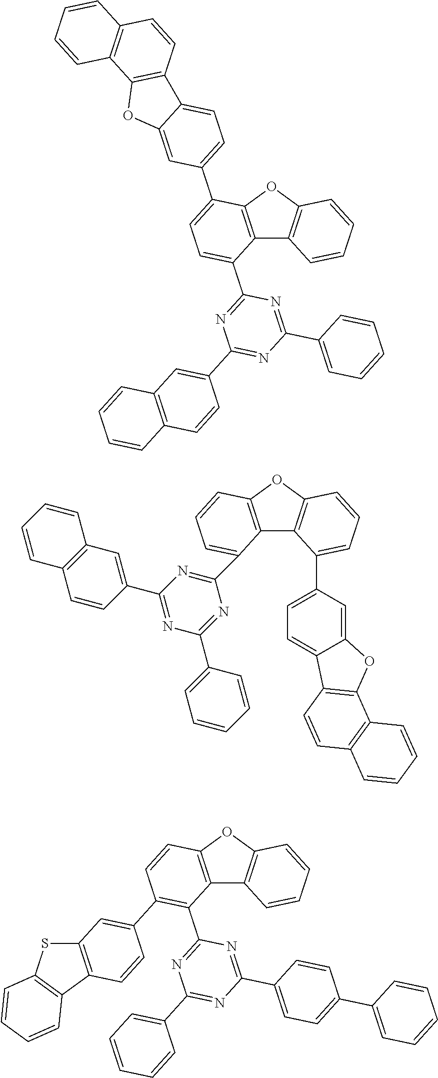 [SMILES: c1ccc(-c2nc(-c3ccc4ccccc4c3)nc(-c3ccc(-c4ccc5c(c4)oc4c6ccccc6ccc54)c4oc5ccccc5c34)n2)cc1 ; c1ccc(-c2nc(-c3ccc4ccccc4c3)nc(-c3cccc4oc5cccc(-c6ccc7c(c6)oc6c8ccccc8ccc76)c5c34)n2)cc1 ; c1ccc(-c2ccc(-c3nc(-c4ccccc4)nc(-c4c(-c5ccc6c(c5)sc5ccccc56)ccc5oc6ccccc6c45)n3)cc2)cc1]

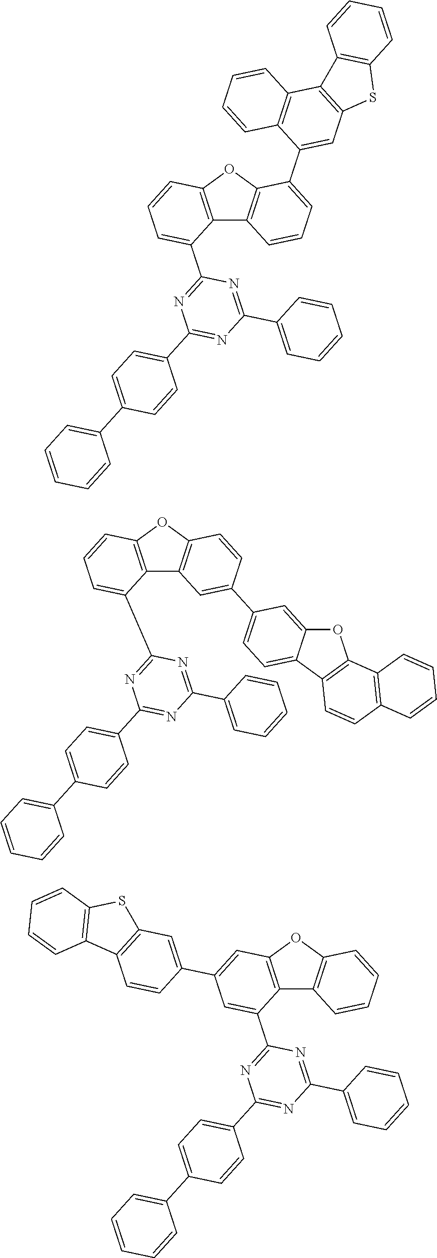

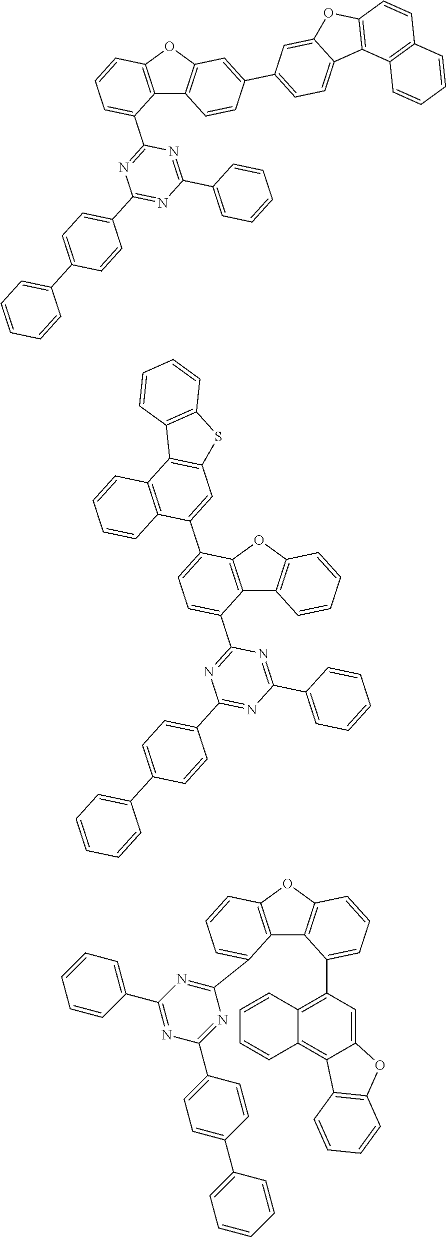 [SMILES: c1ccc(-c2ccc(-c3nc(-c4ccccc4)nc(-c4cccc5oc6cc(-c7ccc8c(c7)oc7ccc9ccccc9c78)ccc6c45)n3)cc2)cc1 ; c1ccc(-c2ccc(-c3nc(-c4ccccc4)nc(-c4ccc(-c5cc6sc7ccccc7c6c6ccccc56)c5oc6ccccc6c45)n3)cc2)cc1 ; c1ccc(-c2ccc(-c3nc(-c4ccccc4)nc(-c4cccc5oc6cccc(-c7cc8oc9ccccc9c8c8ccccc78)c6c45)n3)cc2)cc1]

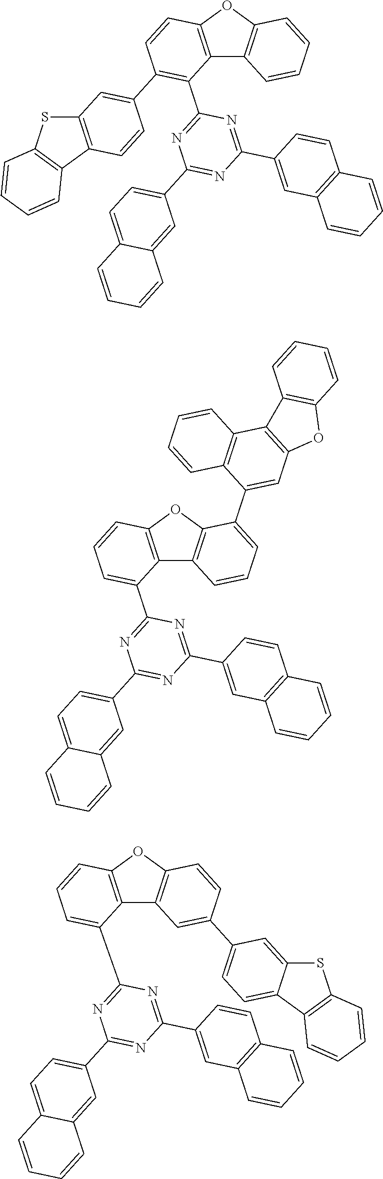 [SMILES: c1ccc2cc(-c3nc(-c4ccc5ccccc5c4)nc(-c4c(-c5ccc6c(c5)sc5ccccc56)ccc5oc6ccccc6c45)n3)ccc2c1 ; c1ccc2cc(-c3nc(-c4ccc5ccccc5c4)nc(-c4cccc5oc6c(-c7cc8oc9ccccc9c8c8ccccc78)cccc6c45)n3)ccc2c1 ; c1ccc2cc(-c3nc(-c4ccc5ccccc5c4)nc(-c4cccc5oc6ccc(-c7ccc8c(c7)sc7ccccc78)cc6c45)n3)ccc2c1]

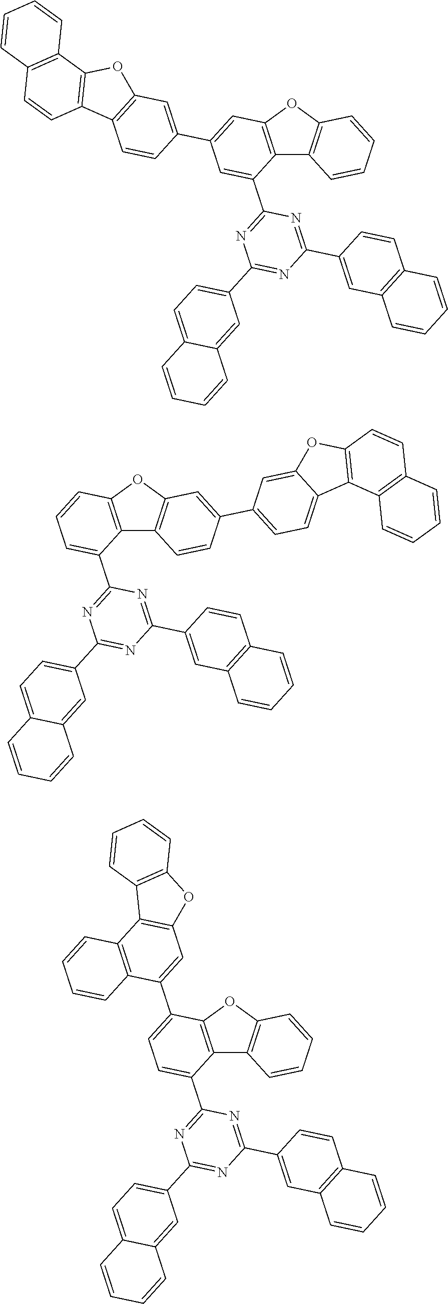 [SMILES: c1ccc2cc(-c3nc(-c4ccc5ccccc5c4)nc(-c4cc(-c5ccc6c(c5)oc5c7ccccc7ccc65)cc5oc6ccccc6c45)n3)ccc2c1 ; c1ccc2cc(-c3nc(-c4ccc5ccccc5c4)nc(-c4cccc5oc6cc(-c7ccc8c(c7)oc7ccc9ccccc9c78)ccc6c45)n3)ccc2c1 ; c1ccc2cc(-c3nc(-c4ccc5ccccc5c4)nc(-c4ccc(-c5cc6oc7ccccc7c6c6ccccc56)c5oc6ccccc6c45)n3)ccc2c1]

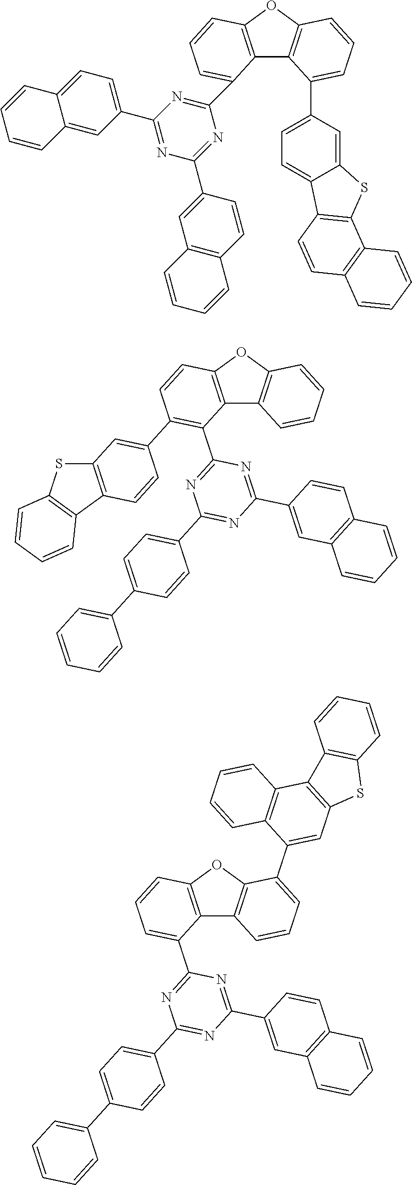 [SMILES: c1ccc2cc(-c3nc(-c4ccc5ccccc5c4)nc(-c4cccc5oc6cccc(-c7ccc8c(c7)sc7c9ccccc9ccc87)c6c45)n3)ccc2c1 ; c1ccc(-c2ccc(-c3nc(-c4ccc5ccccc5c4)nc(-c4c(-c5ccc6c(c5)sc5ccccc56)ccc5oc6ccccc6c45)n3)cc2)cc1 ; c1ccc(-c2ccc(-c3nc(-c4ccc5ccccc5c4)nc(-c4cccc5oc6c(-c7cc8sc9ccccc9c8c8ccccc78)cccc6c45)n3)cc2)cc1]

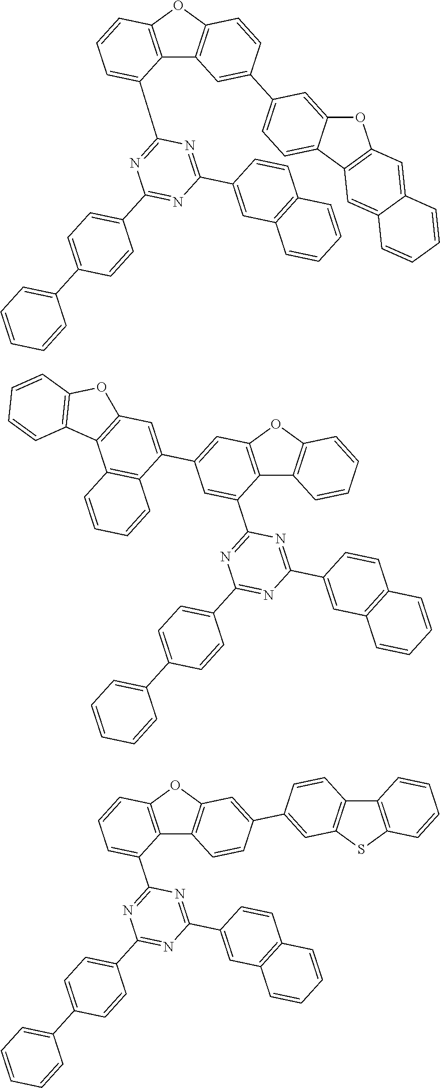

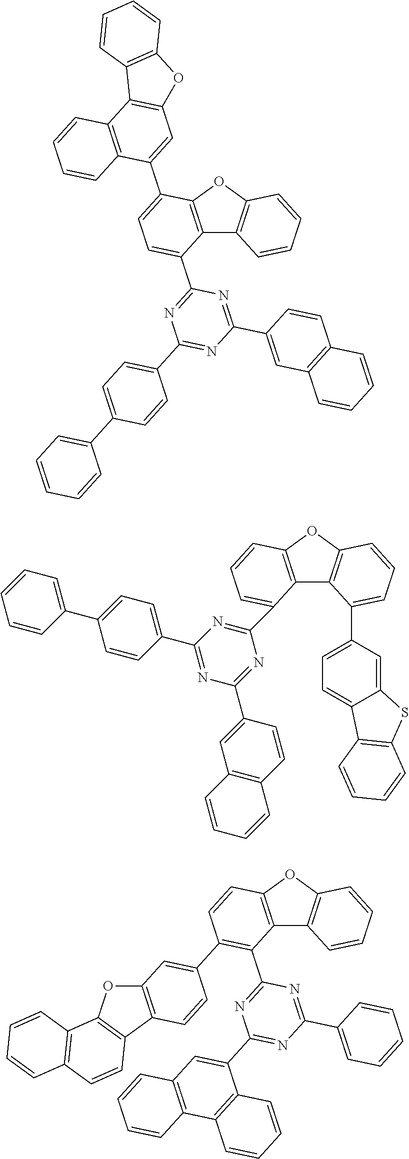 [SMILES: c1ccc(-c2ccc(-c3nc(-c4ccc5ccccc5c4)nc(-c4ccc(-c5cc6oc7ccccc7c6c6ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)sc7ccccc78)c6c45)n3)cc2)cc1 ; c1ccc(-c2nc(-c3cc4ccccc4c4ccccc34)nc(-c3c(-c4ccc5c(c4)oc4c6ccccc6ccc54)ccc4oc5ccccc5c34)n2)cc1]

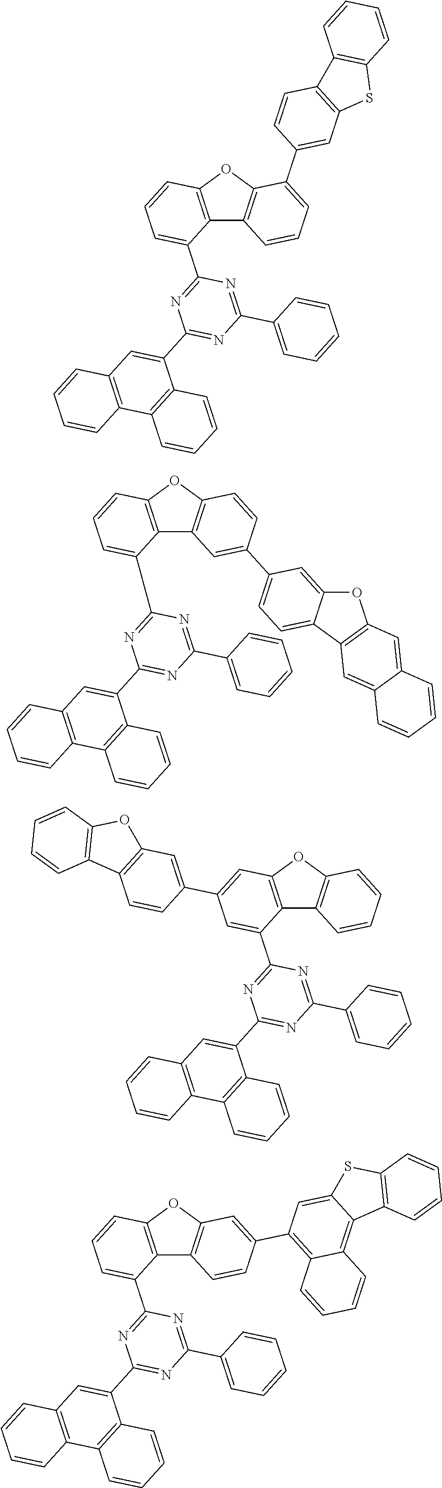

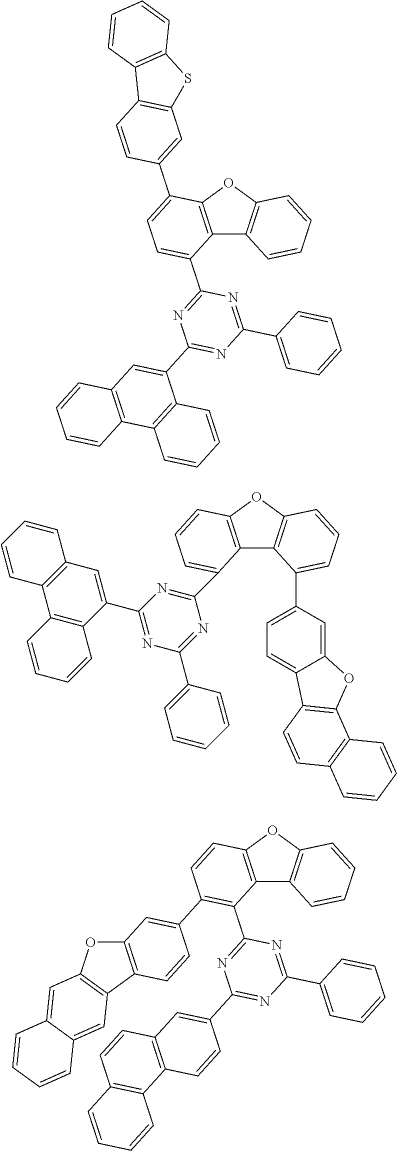

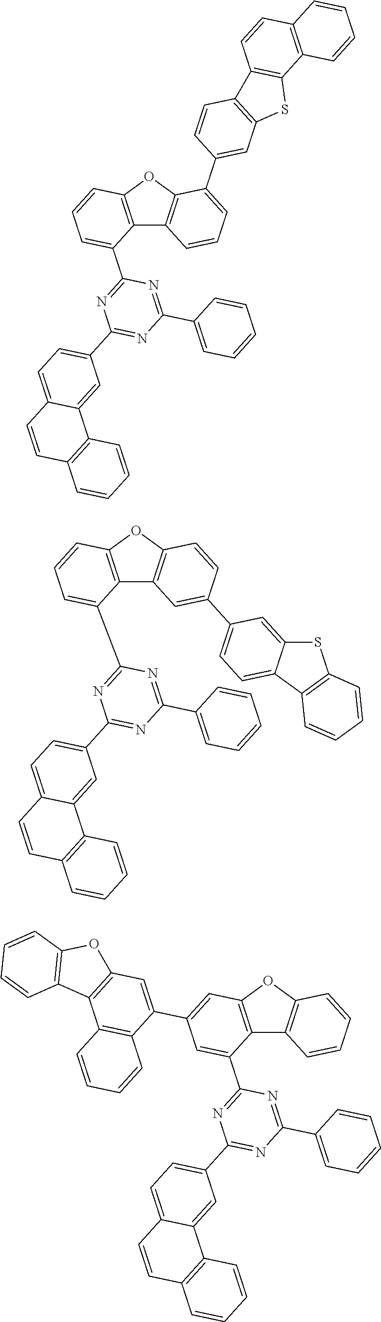

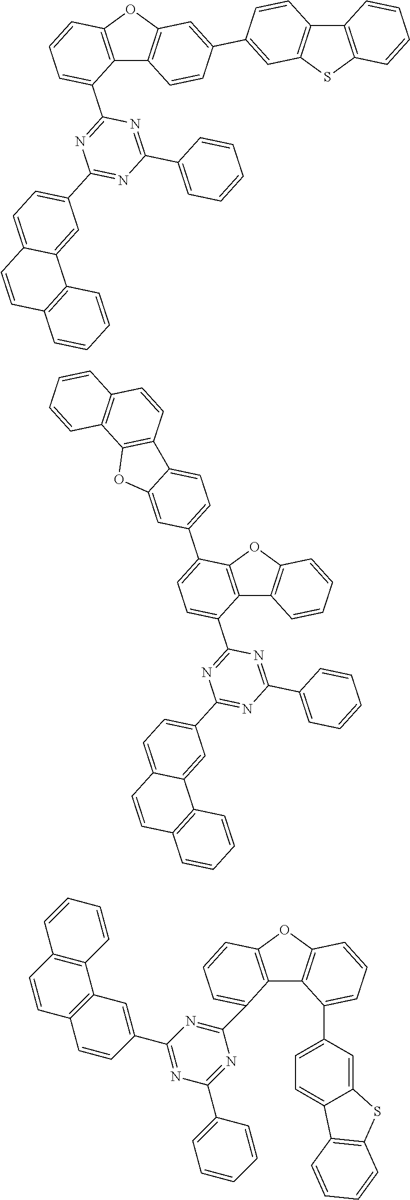

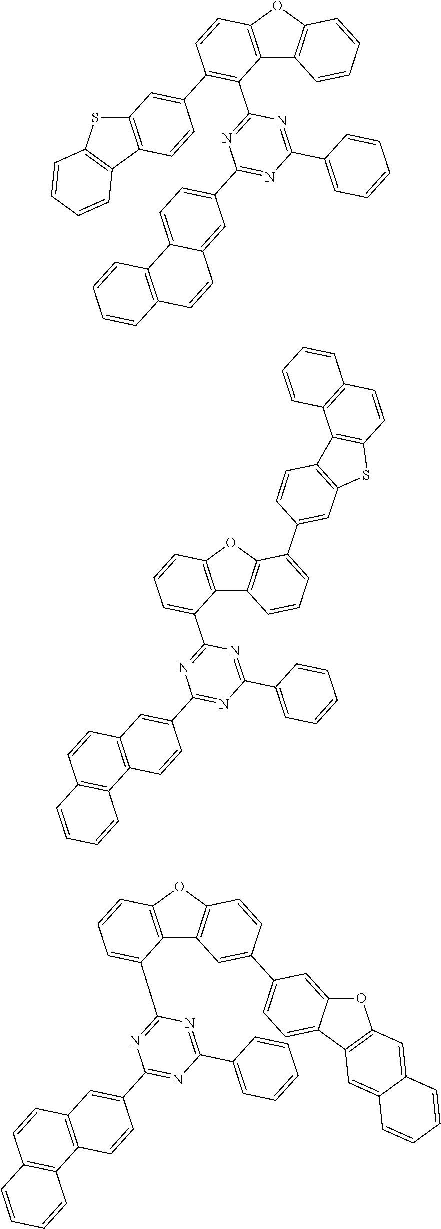 [SMILES: c1ccc(-c2nc(-c3ccc4c(ccc5ccccc54)c3)nc(-c3c(-c4ccc5c(c4)sc4ccccc45)ccc4oc5ccccc5c34)n2)cc1 ; c1ccc(-c2nc(-c3ccc4c(ccc5ccccc54)c3)nc(-c3cccc4oc5c(-c6ccc7c(c6)sc6ccc8ccccc8c67)cccc5c34)n2)cc1 ; c1ccc(-c2nc(-c3ccc4c(ccc5ccccc54)c3)nc(-c3cccc4oc5ccc(-c6ccc7c(c6)oc6cc8ccccc8cc67)cc5c34)n2)cc1]

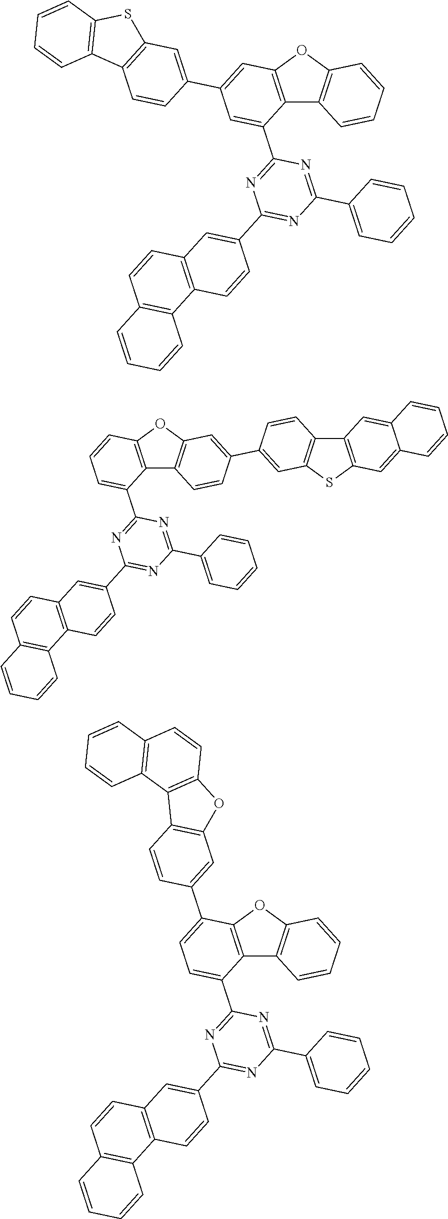 [SMILES: c1ccc(-c2nc(-c3ccc4c(ccc5ccccc54)c3)nc(-c3cc(-c4ccc5c(c4)sc4ccccc45)cc4oc5ccccc5c34)n2)cc1 ; c1ccc(-c2nc(-c3ccc4c(ccc5ccccc54)c3)nc(-c3cccc4oc5cc(-c6ccc7c(c6)sc6cc8ccccc8cc67)ccc5c34)n2)cc1 ; c1ccc(-c2nc(-c3ccc4c(ccc5ccccc54)c3)nc(-c3ccc(-c4ccc5c(c4)oc4ccc6ccccc6c45)c4oc5ccccc5c34)n2)cc1]

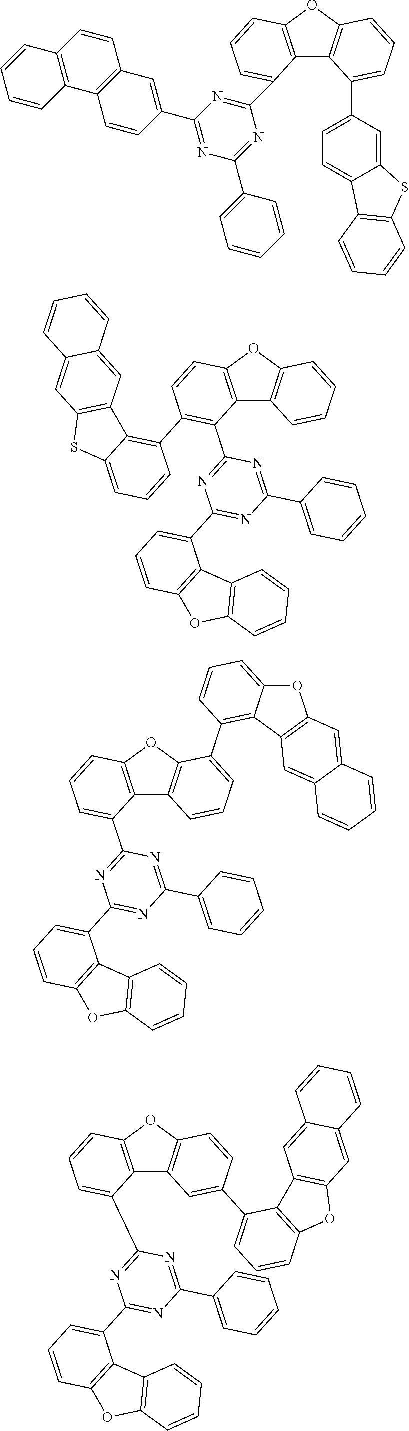

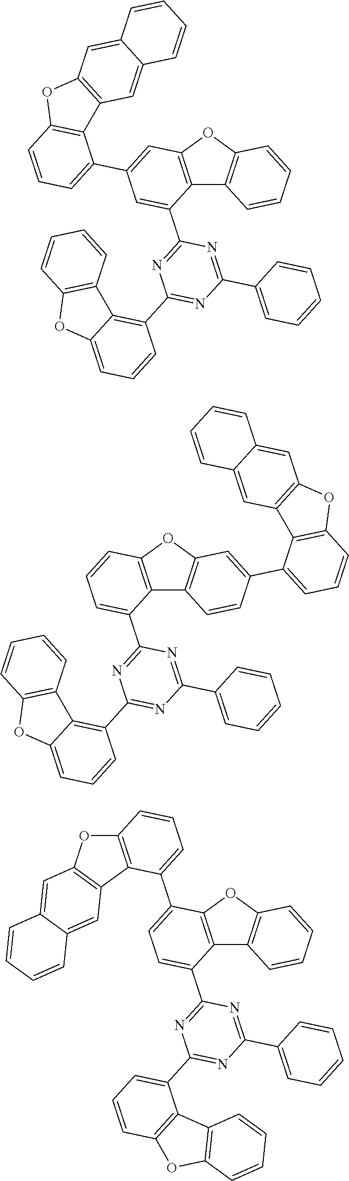 [SMILES: c1ccc(-c2nc(-c3cccc4oc5ccccc5c34)nc(-c3cc(-c4cccc5oc6cc7ccccc7cc6c45)cc4oc5ccccc5c34)n2)cc1 ; c1ccc(-c2nc(-c3cccc4oc5ccccc5c34)nc(-c3cccc4oc5cc(-c6cccc7oc8cc9ccccc9cc8c67)ccc5c34)n2)cc1 ; c1ccc(-c2nc(-c3cccc4oc5ccccc5c34)nc(-c3ccc(-c4cccc5oc6cc7ccccc7cc6c45)c4oc5ccccc5c34)n2)cc1]

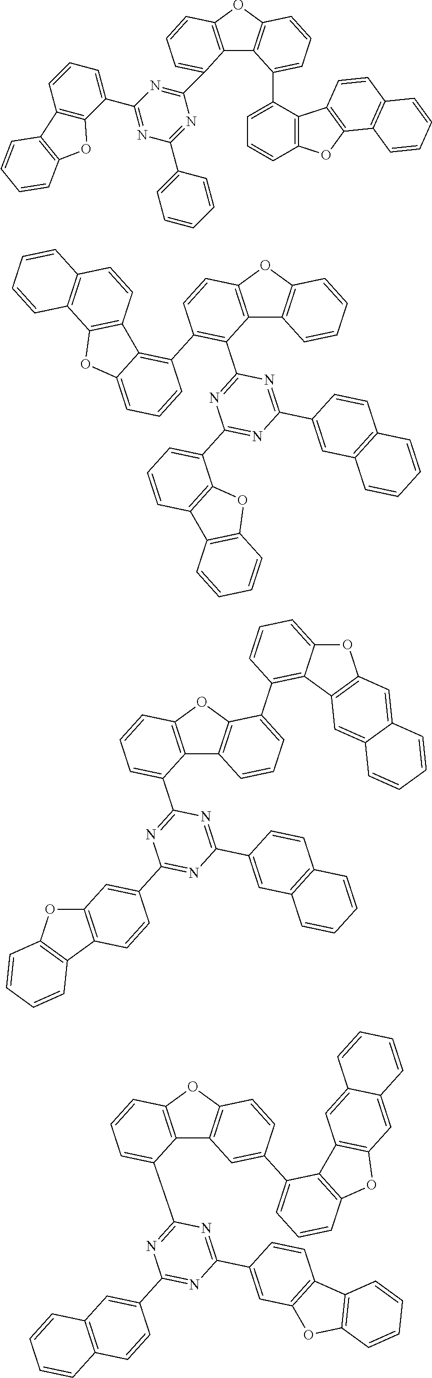

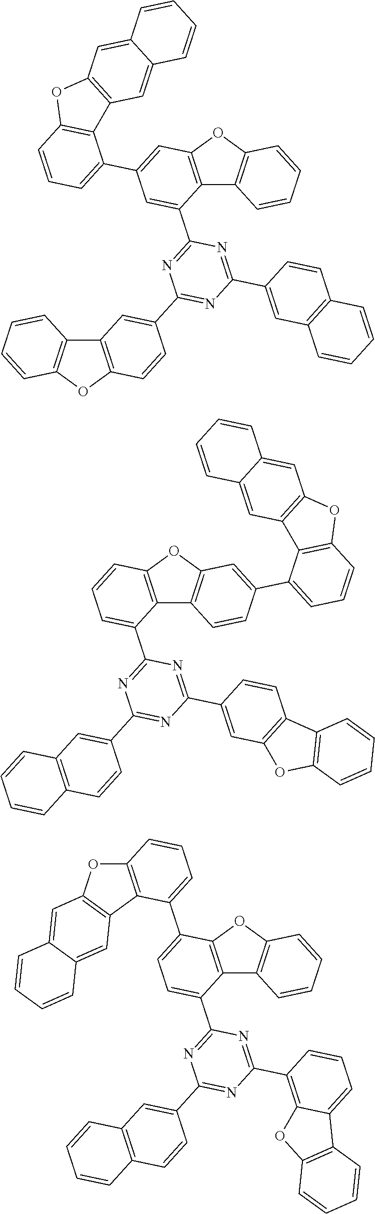 [SMILES: c1ccc2cc(-c3nc(-c4ccc5oc6ccccc6c5c4)nc(-c4cc(-c5cccc6oc7cc8ccccc8cc7c56)cc5oc6ccccc6c45)n3)ccc2c1 ; c1ccc2cc(-c3nc(-c4ccc5c(c4)oc4ccccc45)nc(-c4cccc5oc6cc(-c7cccc8oc9cc%10ccccc%10cc9c78)ccc6c45)n3)ccc2c1 ; c1ccc2cc(-c3nc(-c4cccc5c4oc4ccccc45)nc(-c4ccc(-c5cccc6oc7cc8ccccc8cc7c56)c5oc6ccccc6c45)n3)ccc2c1]

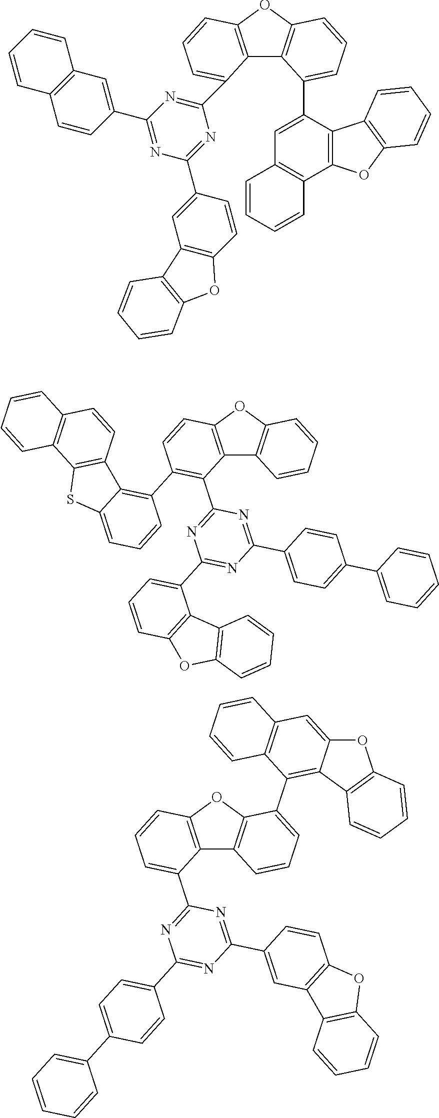

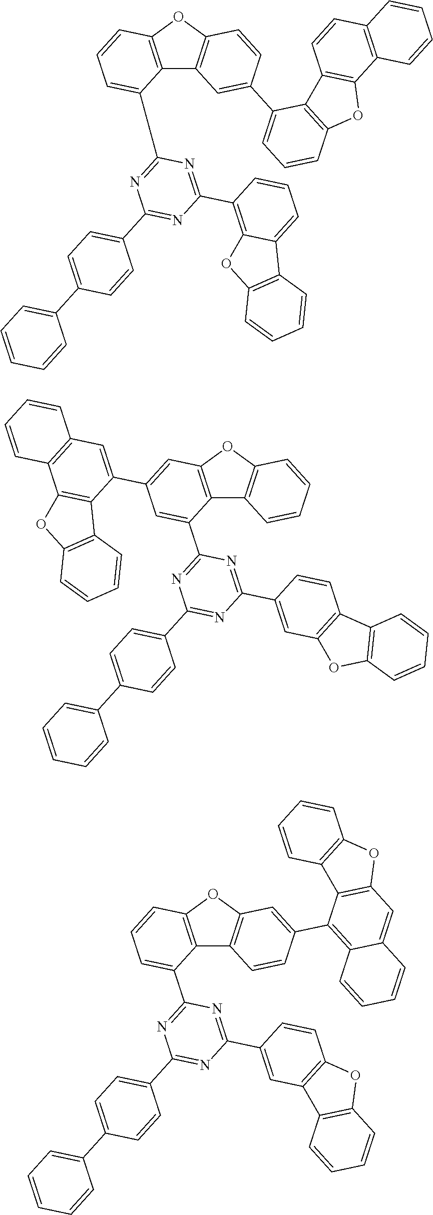

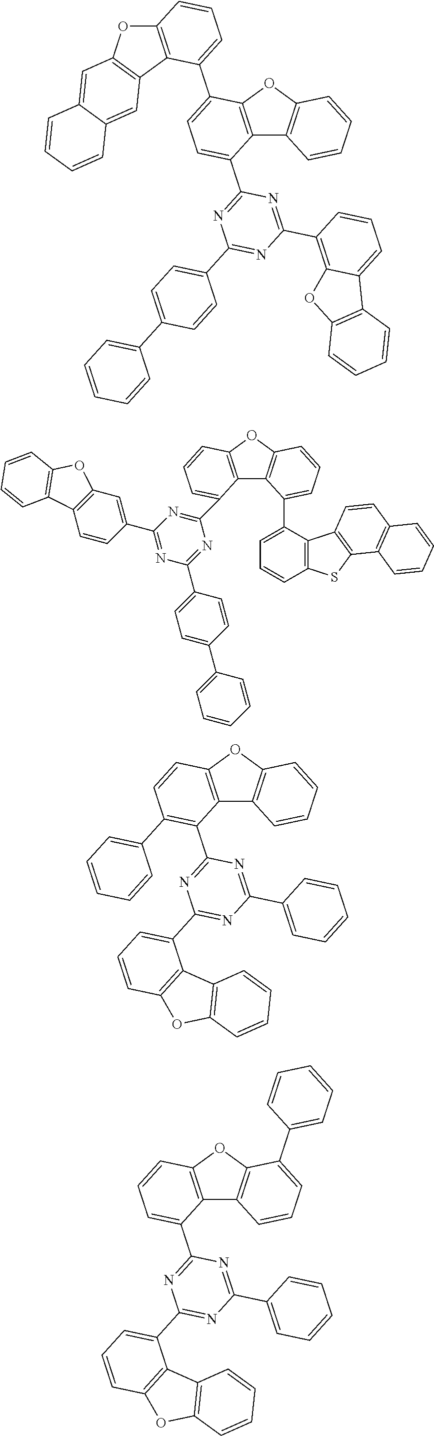

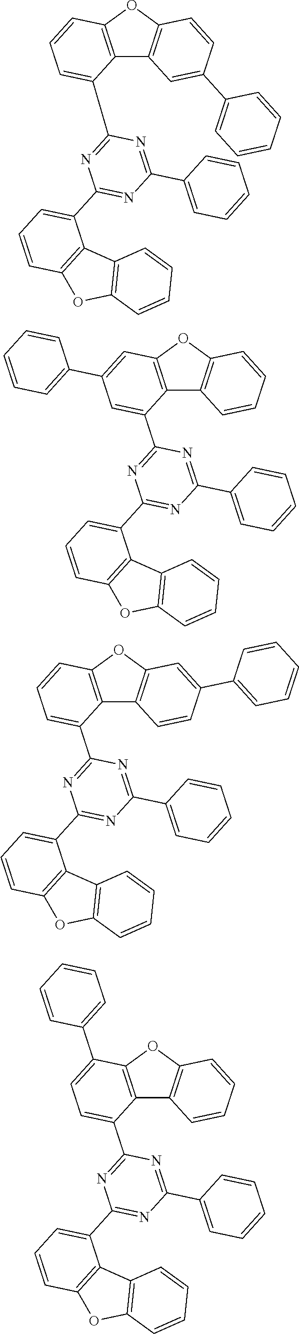

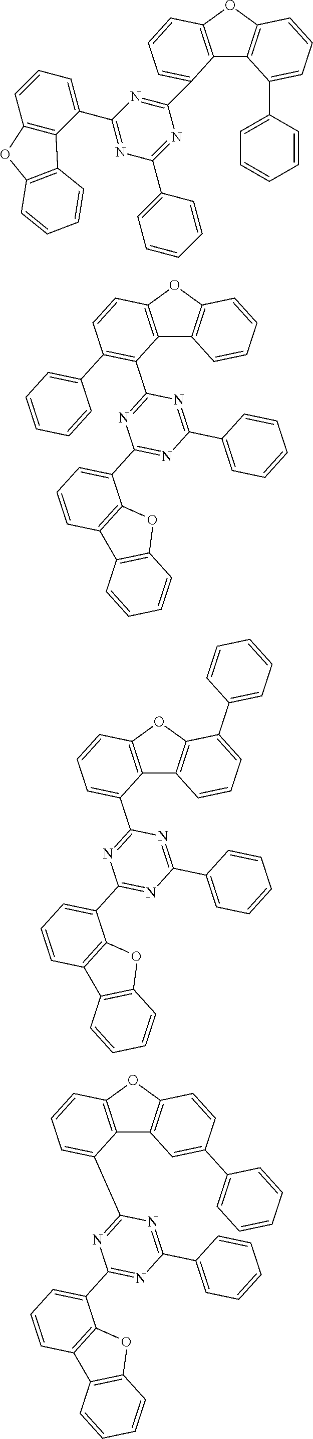

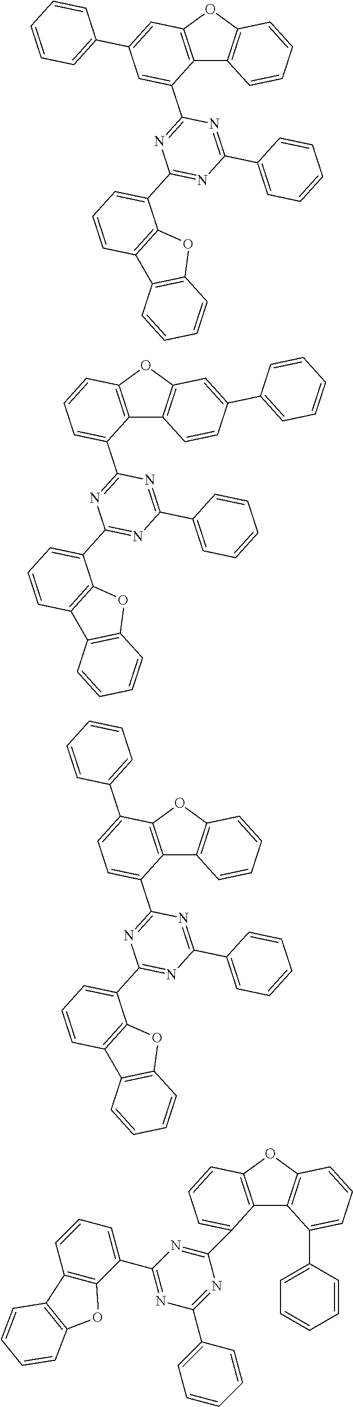

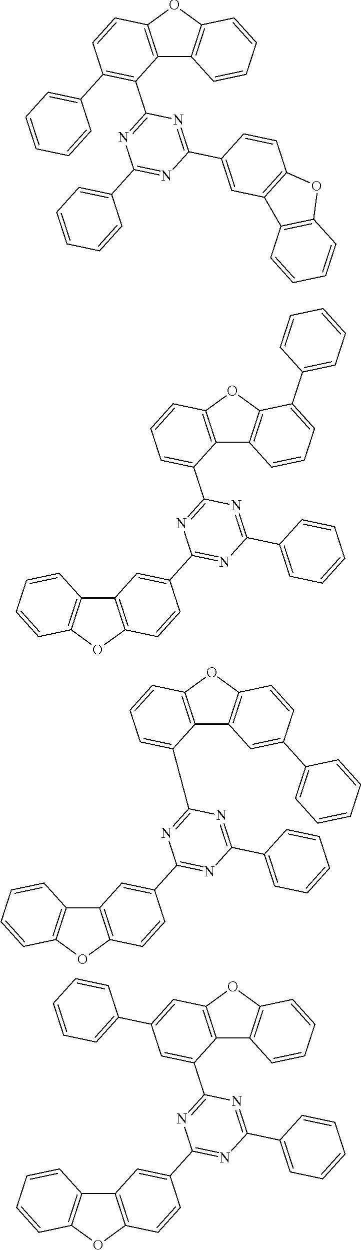

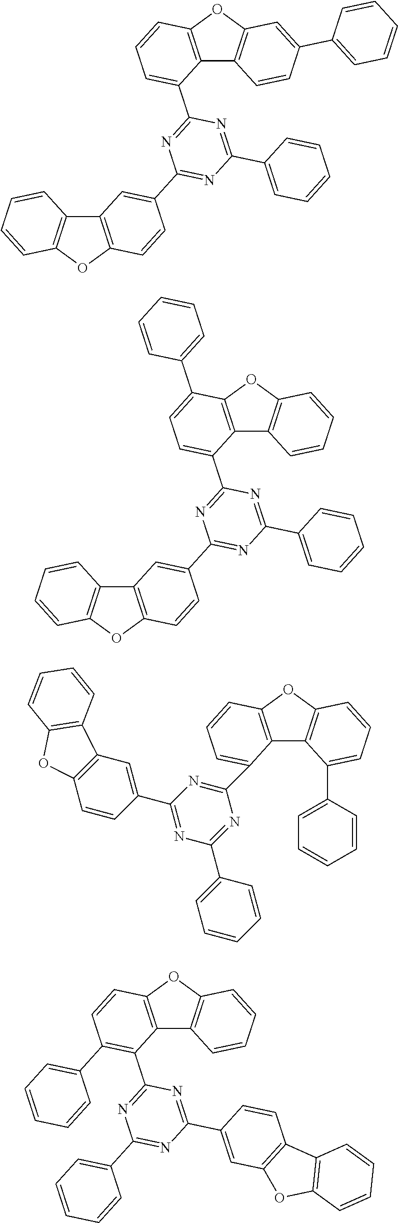

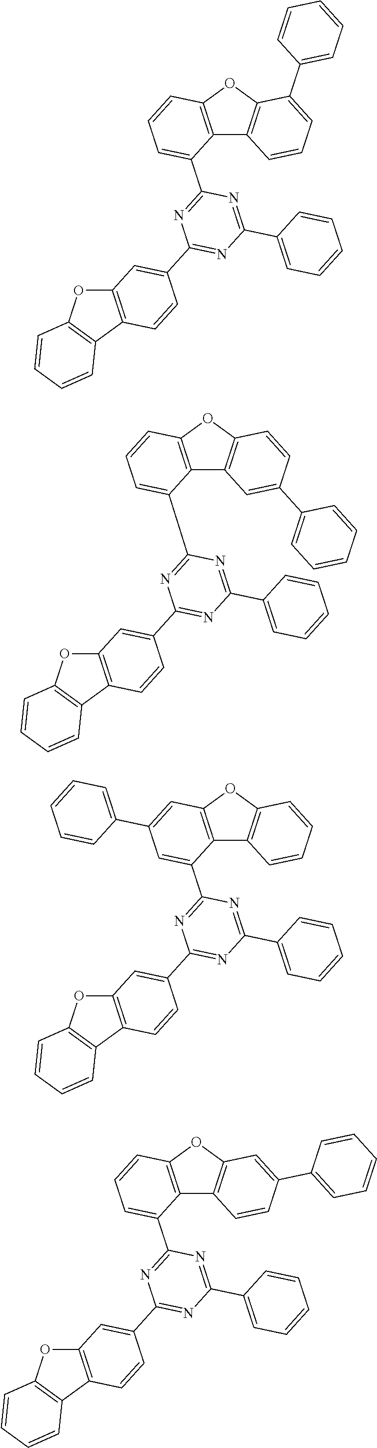

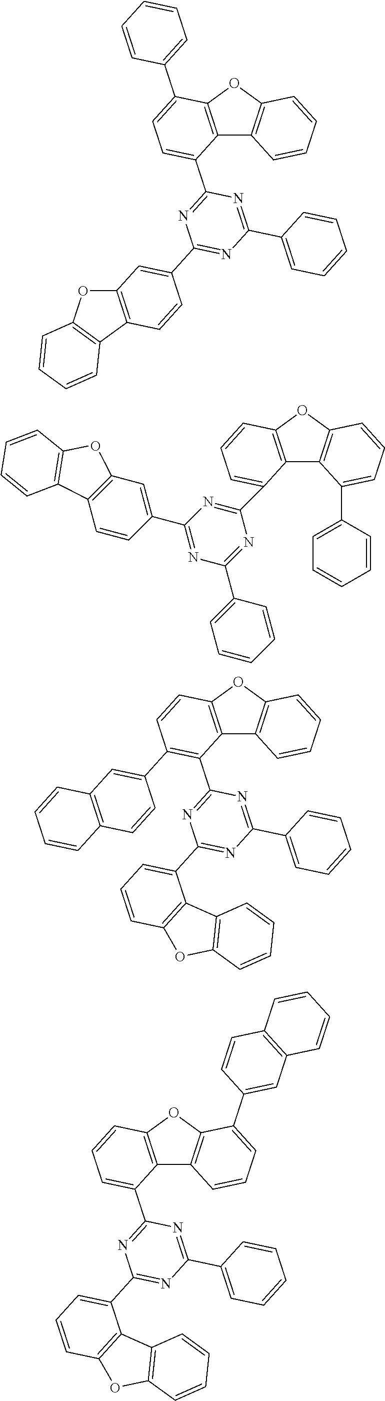

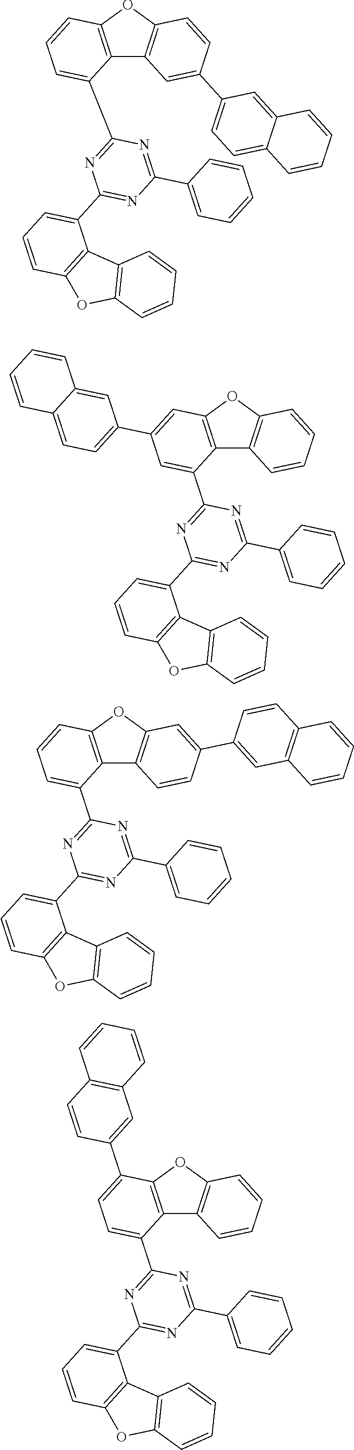

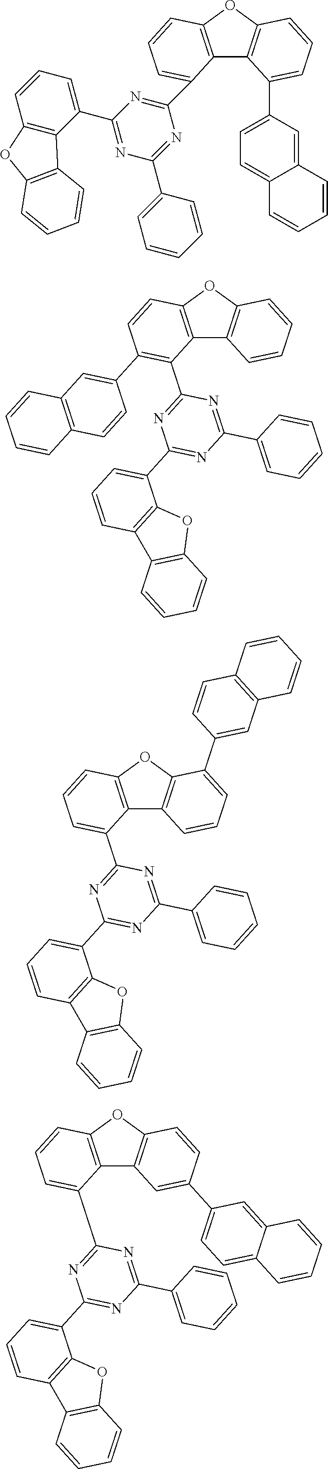

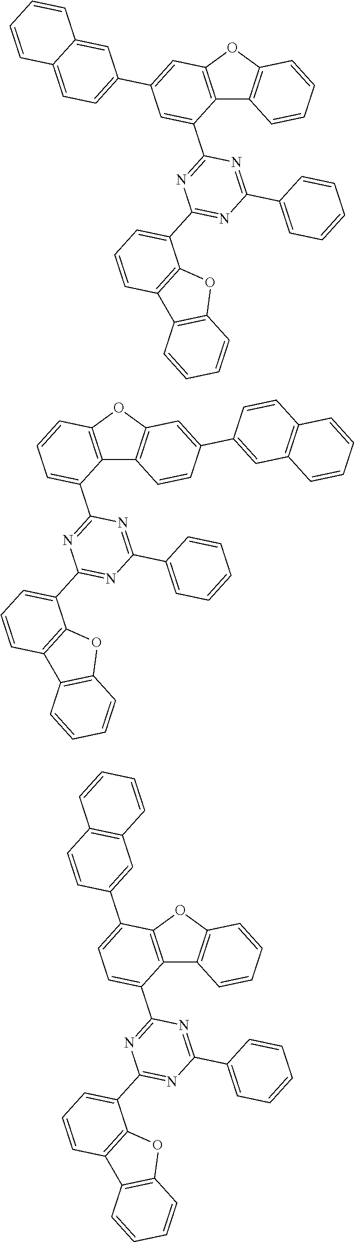

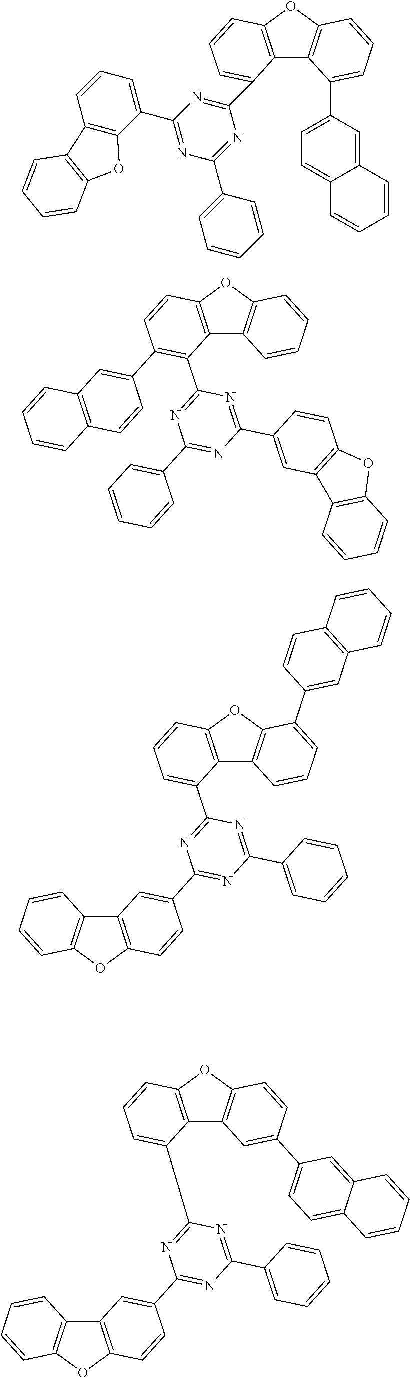

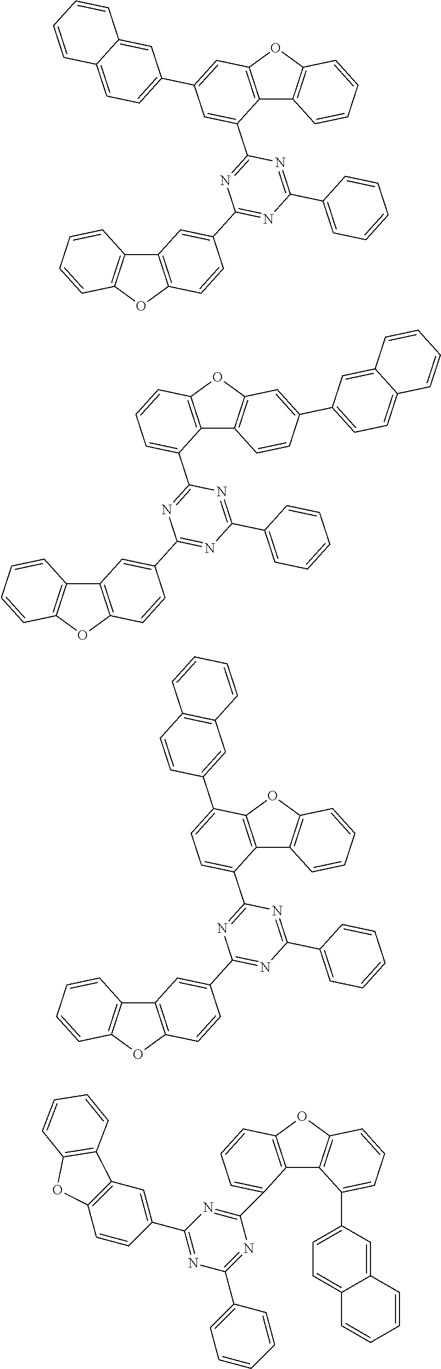 [SMILES: c1ccc(-c2nc(-c3ccc4oc5ccccc5c4c3)nc(-c3cc(-c4ccc5ccccc5c4)cc4oc5ccccc5c34)n2)cc1 ; c1ccc(-c2nc(-c3ccc4oc5ccccc5c4c3)nc(-c3cccc4oc5cc(-c6ccc7ccccc7c6)ccc5c34)n2)cc1 ; c1ccc(-c2nc(-c3ccc4oc5ccccc5c4c3)nc(-c3ccc(-c4ccc5ccccc5c4)c4oc5ccccc5c34)n2)cc1 ; c1ccc(-c2nc(-c3ccc4oc5ccccc5c4c3)nc(-c3cccc4oc5cccc(-c6ccc7ccccc7c6)c5c34)n2)cc1]

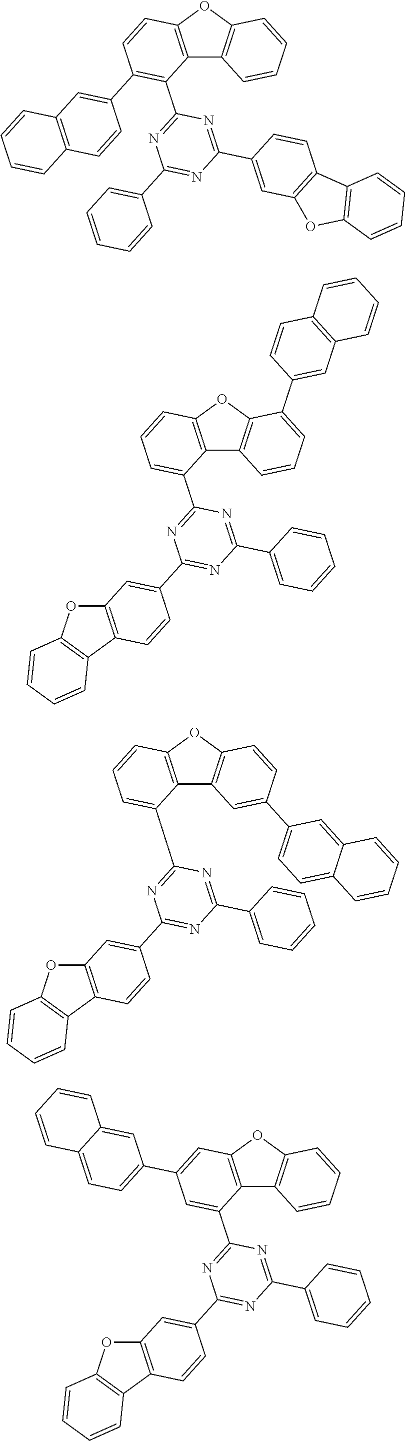

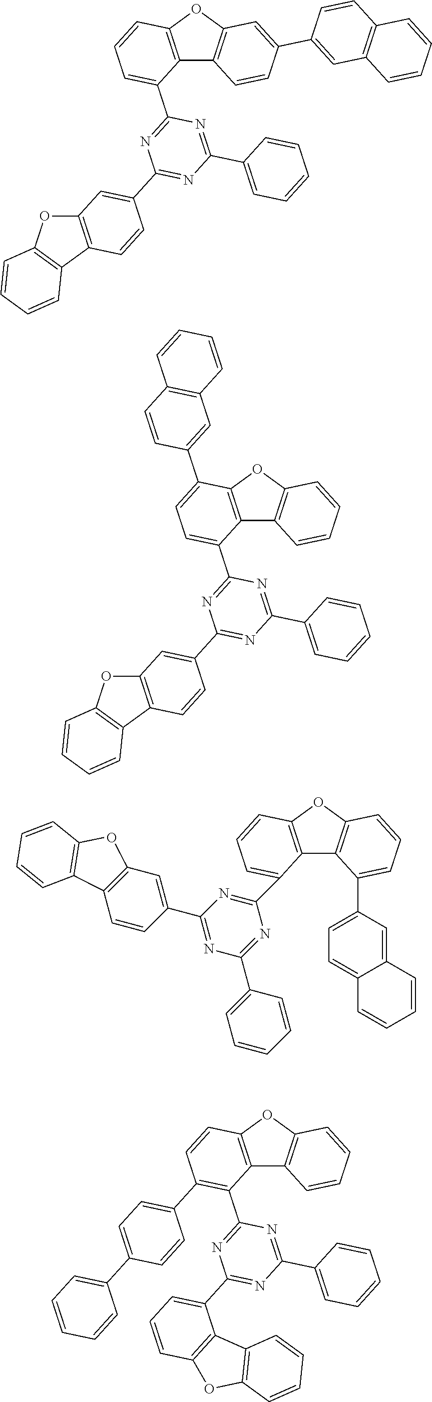 [SMILES: c1ccc(-c2nc(-c3ccc4c(c3)oc3ccccc34)nc(-c3cccc4oc5cc(-c6ccc7ccccc7c6)ccc5c34)n2)cc1 ; c1ccc(-c2nc(-c3ccc4c(c3)oc3ccccc34)nc(-c3ccc(-c4ccc5ccccc5c4)c4oc5ccccc5c34)n2)cc1 ; c1ccc(-c2nc(-c3ccc4c(c3)oc3ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4cccc5oc6ccccc6c45)n3)cc2)cc1]

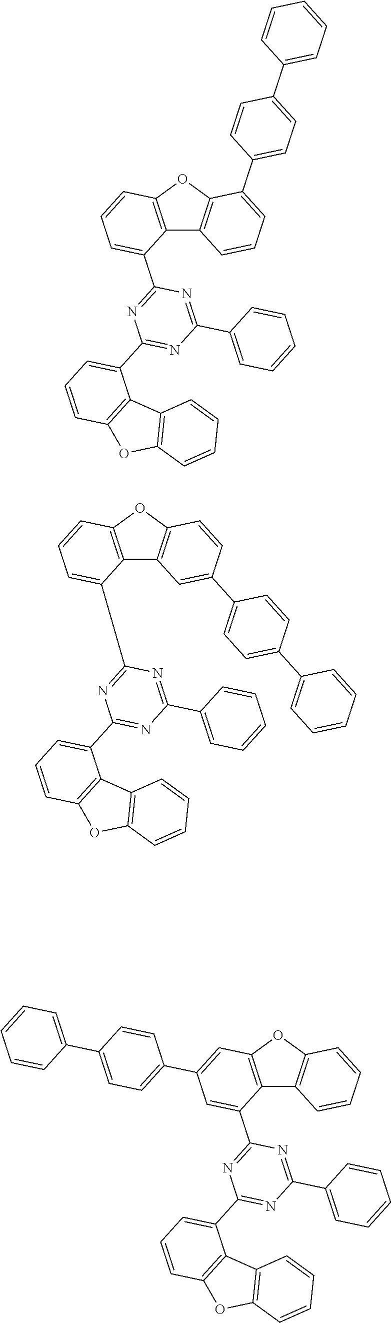 [SMILES: c1ccc(-c2ccc(-c3cccc4c3oc3cccc(-c5nc(-c6ccccc6)nc(-c6cccc7oc8ccccc8c67)n5)c34)cc2)cc1 ; c1ccc(-c2ccc(-c3ccc4oc5cccc(-c6nc(-c7ccccc7)nc(-c7cccc8oc9ccccc9c78)n6)c5c4c3)cc2)cc1 ; c1ccc(-c2ccc(-c3cc(-c4nc(-c5ccccc5)nc(-c5cccc6oc7ccccc7c56)n4)c4c(c3)oc3ccccc34)cc2)cc1]

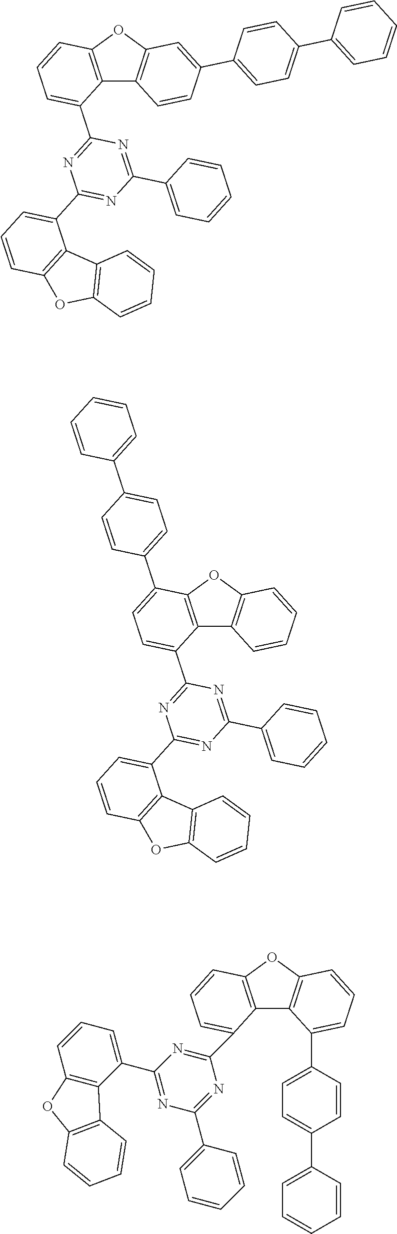 [SMILES: c1ccc(-c2ccc(-c3ccc4c(c3)oc3cccc(-c5nc(-c6ccccc6)nc(-c6cccc7oc8ccccc8c67)n5)c34)cc2)cc1 ; c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2ccc(-c3cccc4oc5cccc(-c6nc(-c7ccccc7)nc(-c7cccc8oc9ccccc9c78)n6)c5c34)cc2)cc1]

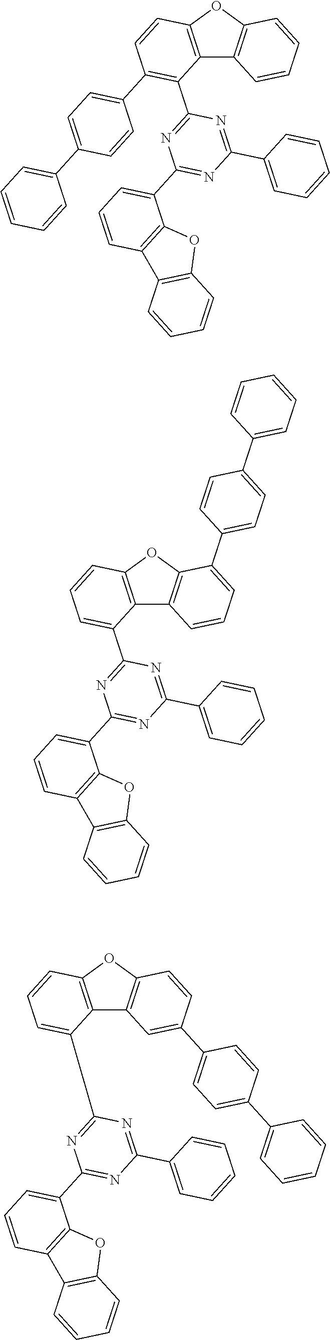 [SMILES: c1ccc(-c2ccc(-c3ccc4oc5ccccc5c4c3-c3nc(-c4ccccc4)nc(-c4cccc5c4oc4ccccc45)n3)cc2)cc1 ; c1ccc(-c2ccc(-c3cccc4c3oc3cccc(-c5nc(-c6ccccc6)nc(-c6cccc7c6oc6ccccc67)n5)c34)cc2)cc1 ; c1ccc(-c2ccc(-c3ccc4oc5cccc(-c6nc(-c7ccccc7)nc(-c7cccc8c7oc7ccccc78)n6)c5c4c3)cc2)cc1]

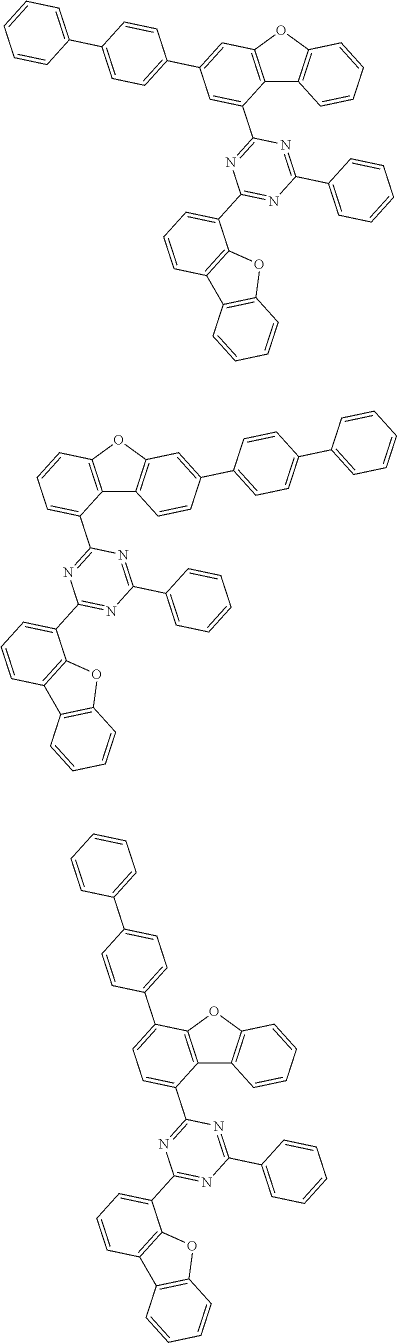 [SMILES: c1ccc(-c2ccc(-c3cc(-c4nc(-c5ccccc5)nc(-c5cccc6c5oc5ccccc56)n4)c4c(c3)oc3ccccc34)cc2)cc1 ; c1ccc(-c2ccc(-c3ccc4c(c3)oc3cccc(-c5nc(-c6ccccc6)nc(-c6cccc7c6oc6ccccc67)n5)c34)cc2)cc1 ; c1ccc(-c2ccc(-c3ccc(-c4nc(-c5ccccc5)nc(-c5cccc6c5oc5ccccc56)n4)c4c3oc3ccccc34)cc2)cc1]

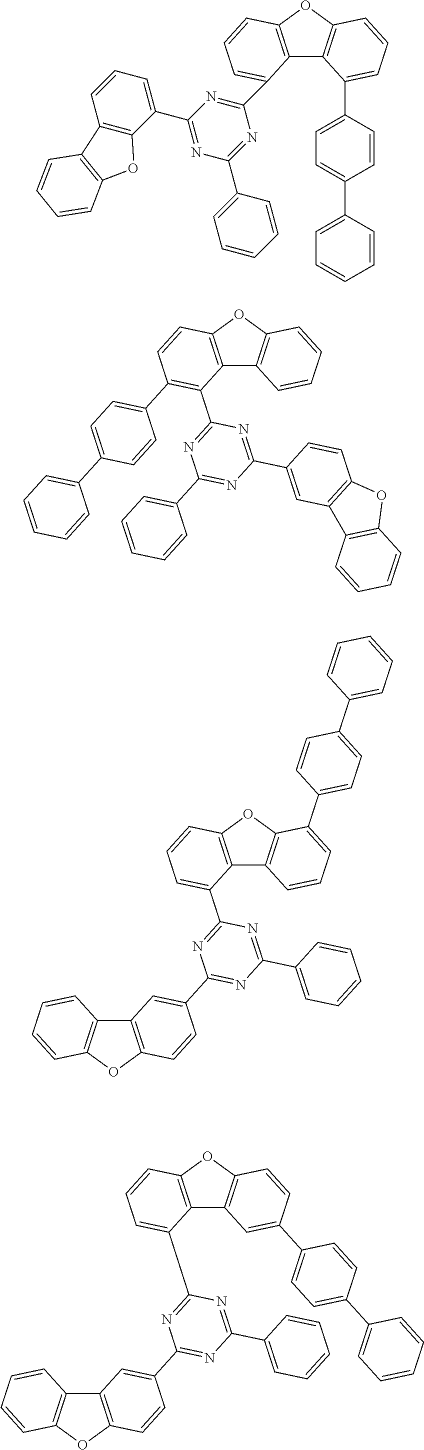

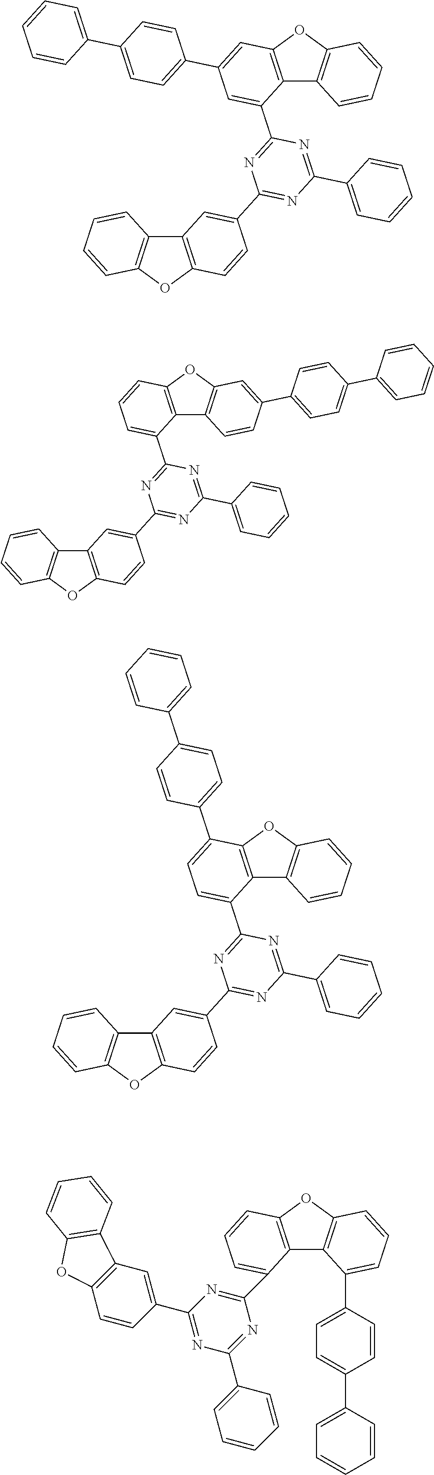 [SMILES: c1ccc(-c2ccc(-c3cc(-c4nc(-c5ccccc5)nc(-c5ccc6oc7ccccc7c6c5)n4)c4c(c3)oc3ccccc34)cc2)cc1 ; c1ccc(-c2ccc(-c3ccc4c(c3)oc3cccc(-c5nc(-c6ccccc6)nc(-c6ccc7oc8ccccc8c7c6)n5)c34)cc2)cc1 ; c1ccc(-c2ccc(-c3ccc(-c4nc(-c5ccccc5)nc(-c5ccc6oc7ccccc7c6c5)n4)c4c3oc3ccccc34)cc2)cc1 ; c1ccc(-c2ccc(-c3cccc4oc5cccc(-c6nc(-c7ccccc7)nc(-c7ccc8oc9ccccc9c8c7)n6)c5c34)cc2)cc1]

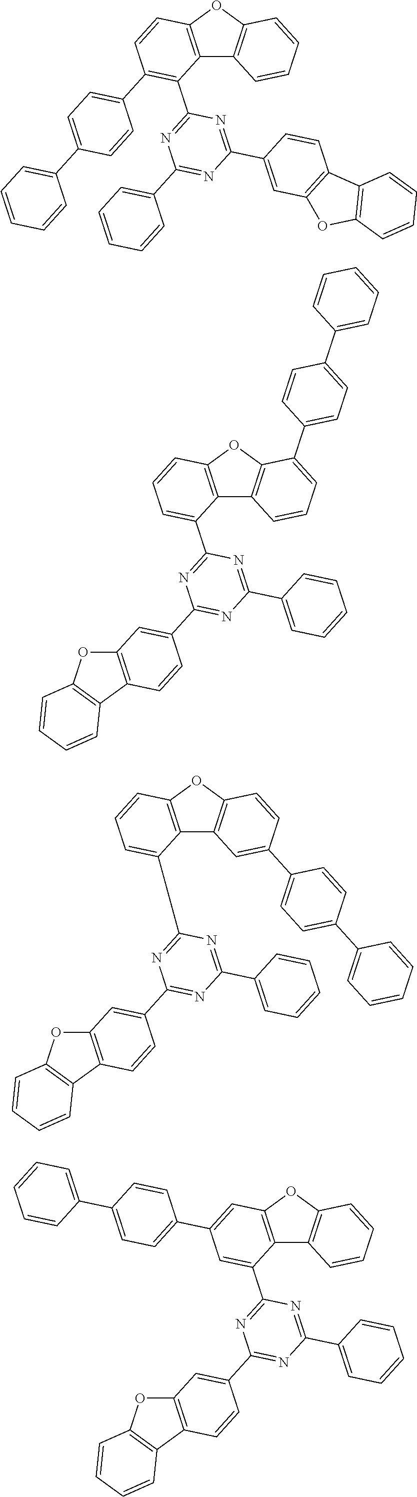

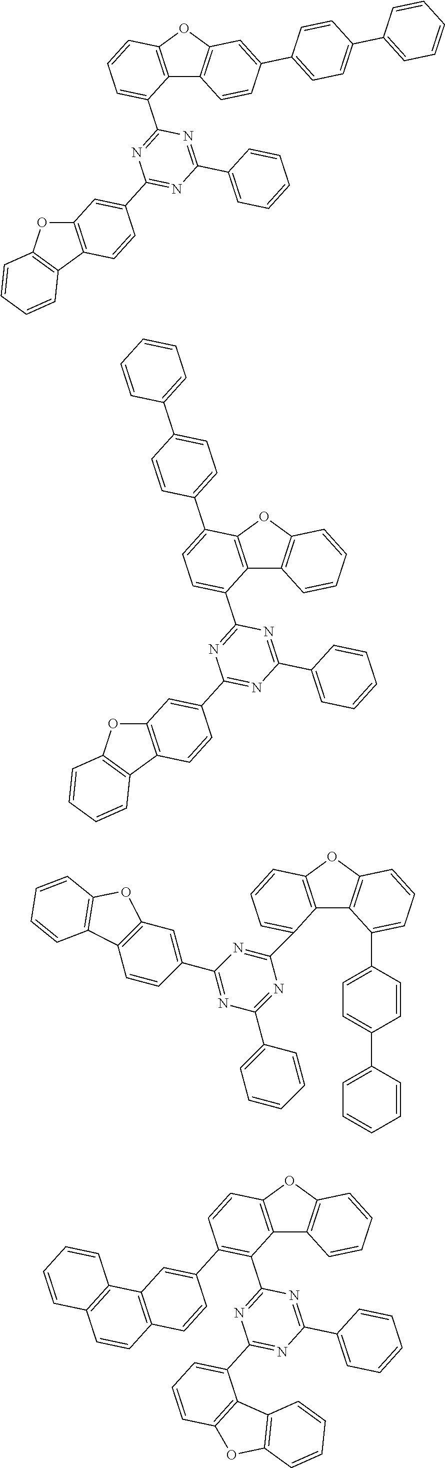 [SMILES: c1ccc(-c2ccc(-c3ccc4c(c3)oc3cccc(-c5nc(-c6ccccc6)nc(-c6ccc7c(c6)oc6ccccc67)n5)c34)cc2)cc1 ; c1ccc(-c2ccc(-c3ccc(-c4nc(-c5ccccc5)nc(-c5ccc6c(c5)oc5ccccc56)n4)c4c3oc3ccccc34)cc2)cc1 ; c1ccc(-c2ccc(-c3cccc4oc5cccc(-c6nc(-c7ccccc7)nc(-c7ccc8c(c7)oc7ccccc78)n6)c5c34)cc2)cc1 ; c1ccc(-c2nc(-c3cccc4oc5ccccc5c34)nc(-c3c(-c4ccc5ccc6ccccc6c5c4)ccc4oc5ccccc5c34)n2)cc1]

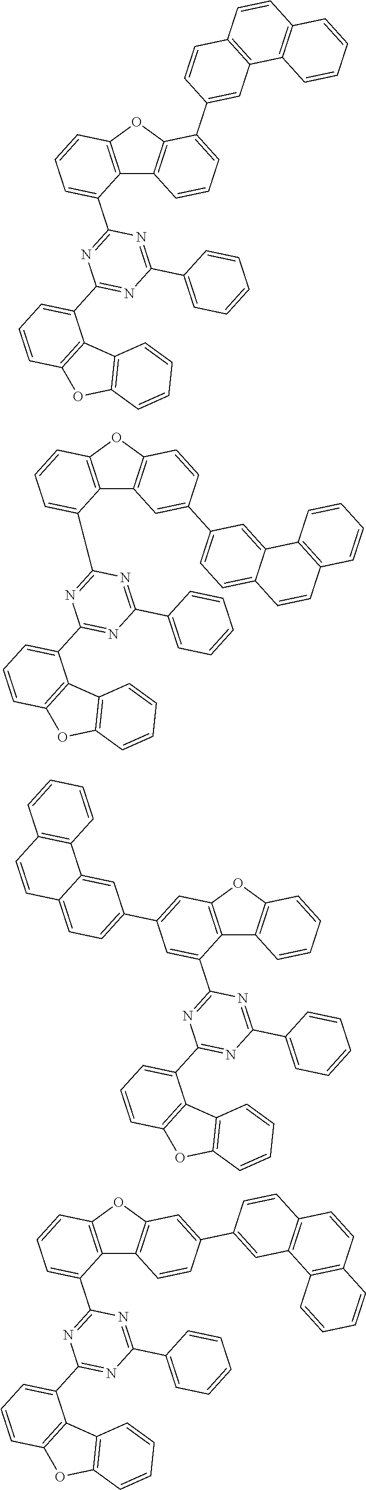

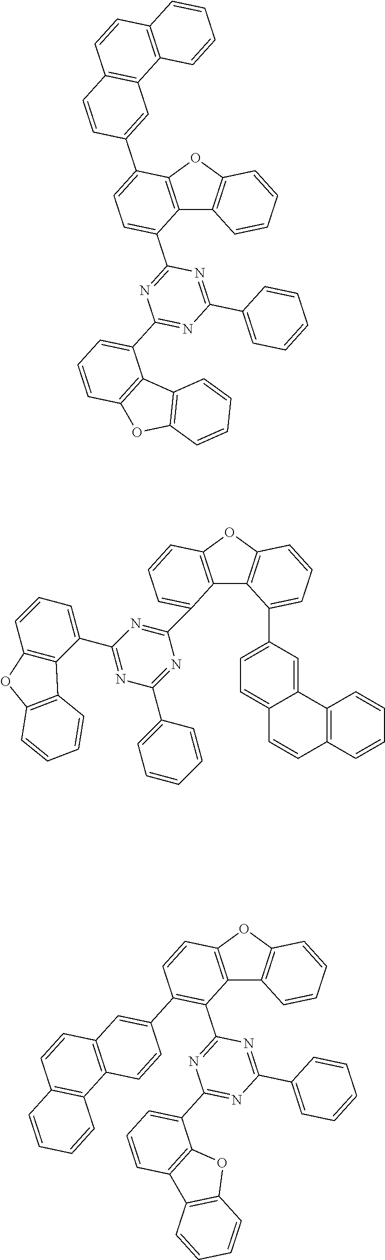 [SMILES: c1ccc(-c2nc(-c3cccc4oc5ccccc5c34)nc(-c3ccc(-c4ccc5ccc6ccccc6c5c4)c4oc5ccccc5c34)n2)cc1 ; c1ccc(-c2nc(-c3cccc4oc5ccccc5c34)nc(-c3cccc4oc5cccc(-c6ccc7ccc8ccccc8c7c6)c5c34)n2)cc1 ; c1ccc(-c2nc(-c3cccc4c3oc3ccccc34)nc(-c3c(-c4ccc5c(ccc6ccccc65)c4)ccc4oc5ccccc5c34)n2)cc1]

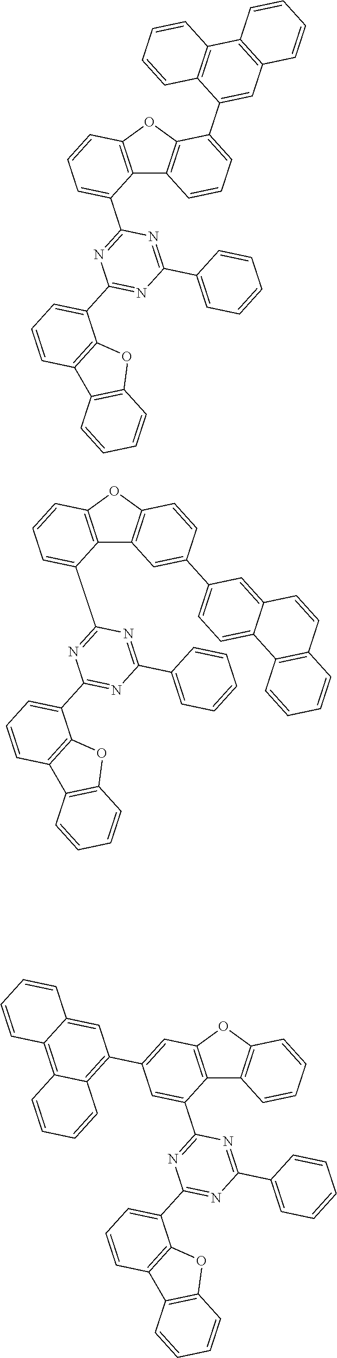

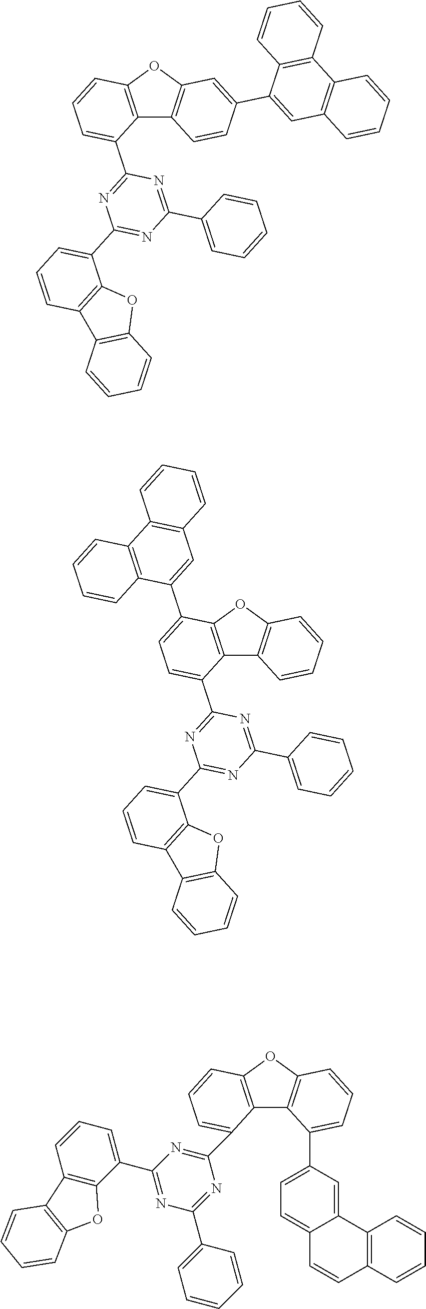 [SMILES: c1ccc(-c2nc(-c3cccc4c3oc3ccccc34)nc(-c3cccc4oc5cc(-c6cc7ccccc7c7ccccc67)ccc5c34)n2)cc1 ; c1ccc(-c2nc(-c3cccc4c3oc3ccccc34)nc(-c3ccc(-c4cc5ccccc5c5ccccc45)c4oc5ccccc5c34)n2)cc1 ; c1ccc(-c2nc(-c3cccc4c3oc3ccccc34)nc(-c3cccc4oc5cccc(-c6ccc7ccc8ccccc8c7c6)c5c34)n2)cc1]

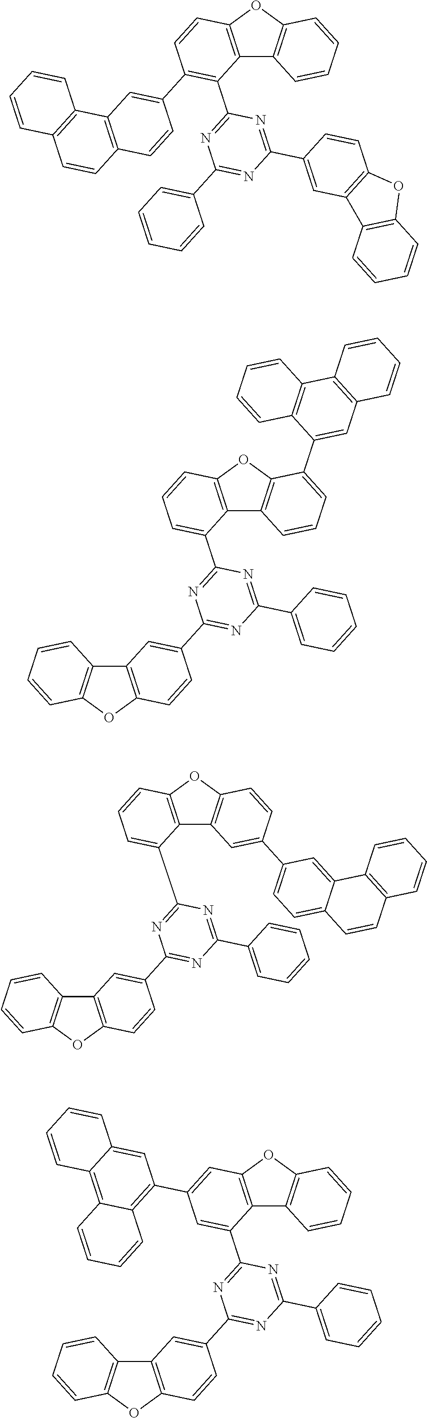 [SMILES: c1ccc(-c2nc(-c3ccc4oc5ccccc5c4c3)nc(-c3c(-c4ccc5ccc6ccccc6c5c4)ccc4oc5ccccc5c34)n2)cc1 ; c1ccc(-c2nc(-c3ccc4oc5ccccc5c4c3)nc(-c3cccc4oc5c(-c6cc7ccccc7c7ccccc67)cccc5c34)n2)cc1 ; c1ccc(-c2nc(-c3ccc4oc5ccccc5c4c3)nc(-c3cccc4oc5ccc(-c6ccc7ccc8ccccc8c7c6)cc5c34)n2)cc1 ; c1ccc(-c2nc(-c3ccc4oc5ccccc5c4c3)nc(-c3cc(-c4cc5ccccc5c5ccccc45)cc4oc5ccccc5c34)n2)cc1]

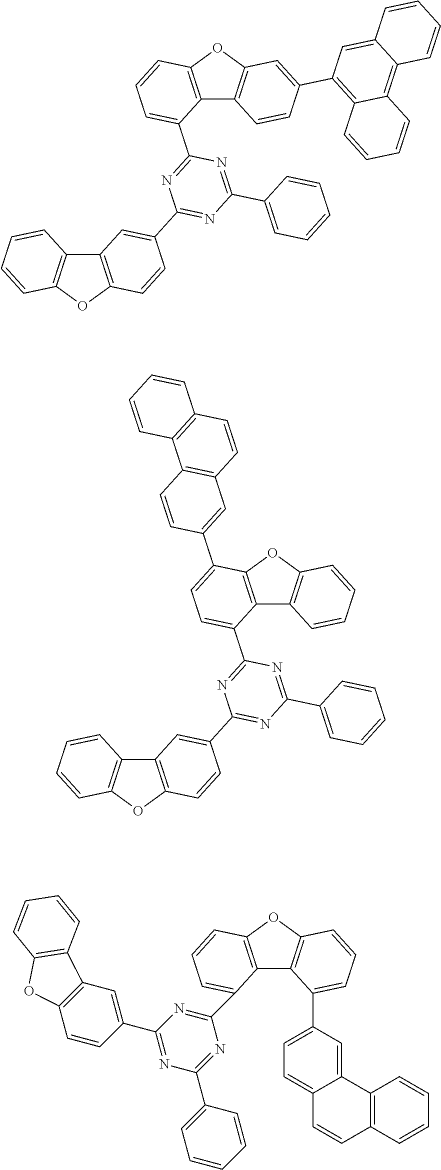 [SMILES: c1ccc(-c2nc(-c3ccc4oc5ccccc5c4c3)nc(-c3cccc4oc5cc(-c6cc7ccccc7c7ccccc67)ccc5c34)n2)cc1 ; c1ccc(-c2nc(-c3ccc4oc5ccccc5c4c3)nc(-c3ccc(-c4ccc5c(ccc6ccccc65)c4)c4oc5ccccc5c34)n2)cc1 ; c1ccc(-c2nc(-c3ccc4oc5ccccc5c4c3)nc(-c3cccc4oc5cccc(-c6ccc7ccc8ccccc8c7c6)c5c34)n2)cc1]

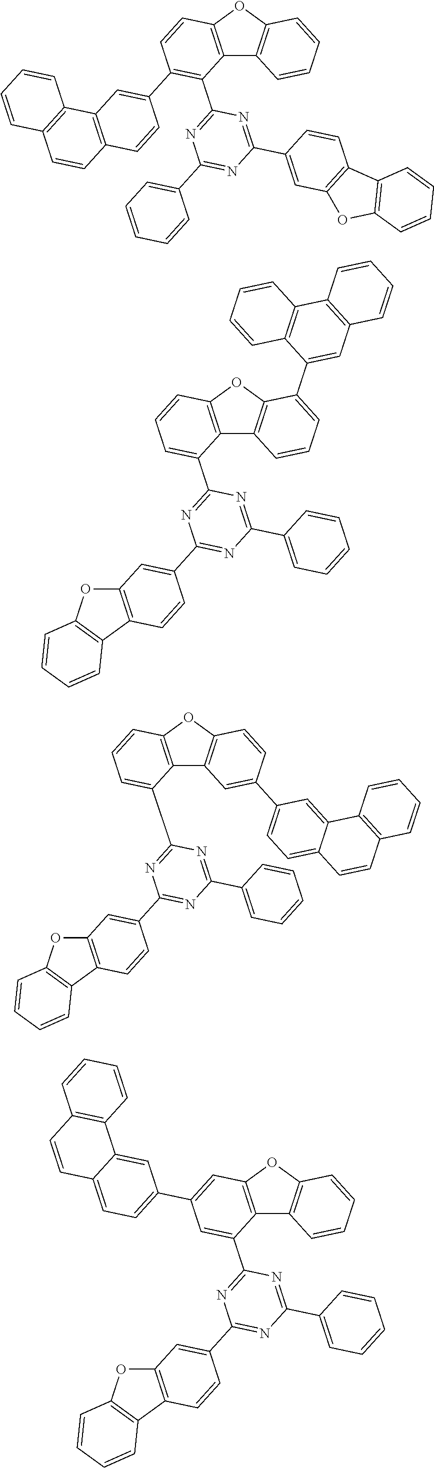

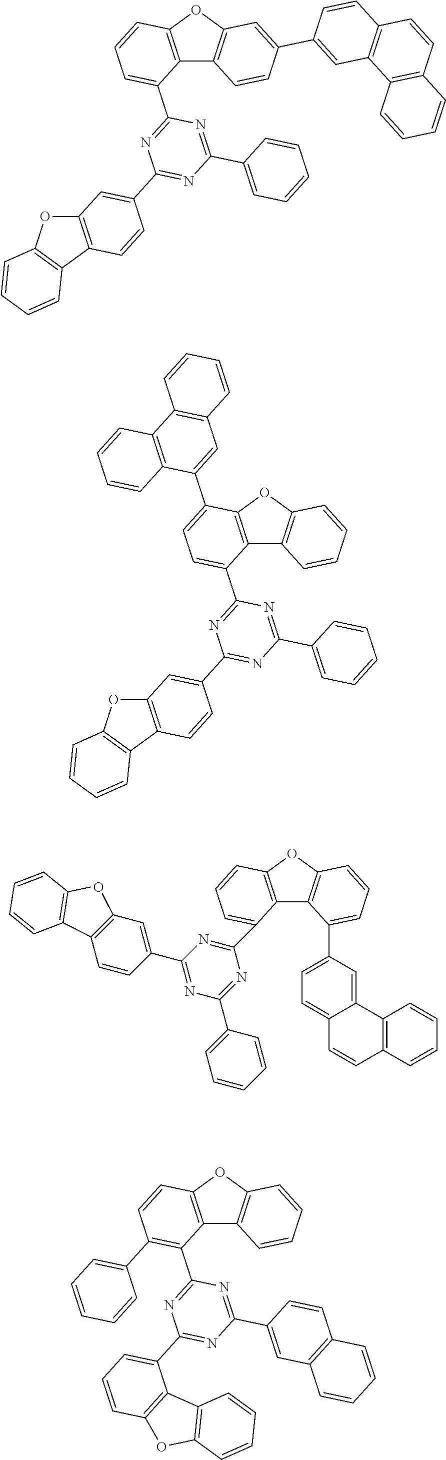 [SMILES: c1ccc(-c2nc(-c3ccc4c(c3)oc3ccccc34)nc(-c3cccc4oc5cc(-c6ccc7ccc8ccccc8c7c6)ccc5c34)n2)cc1 ; c1ccc(-c2nc(-c3ccc4c(c3)oc3ccccc34)nc(-c3ccc(-c4cc5ccccc5c5ccccc45)c4oc5ccccc5c34)n2)cc1 ; c1ccc(-c2nc(-c3ccc4c(c3)oc3ccccc34)nc(-c3cccc4oc5cccc(-c6ccc7ccc8ccccc8c7c6)c5c34)n2)cc1 ; c1ccc(-c2ccc3oc4ccccc4c3c2-c2nc(-c3ccc4ccccc4c3)nc(-c3cccc4oc5ccccc5c34)n2)cc1]

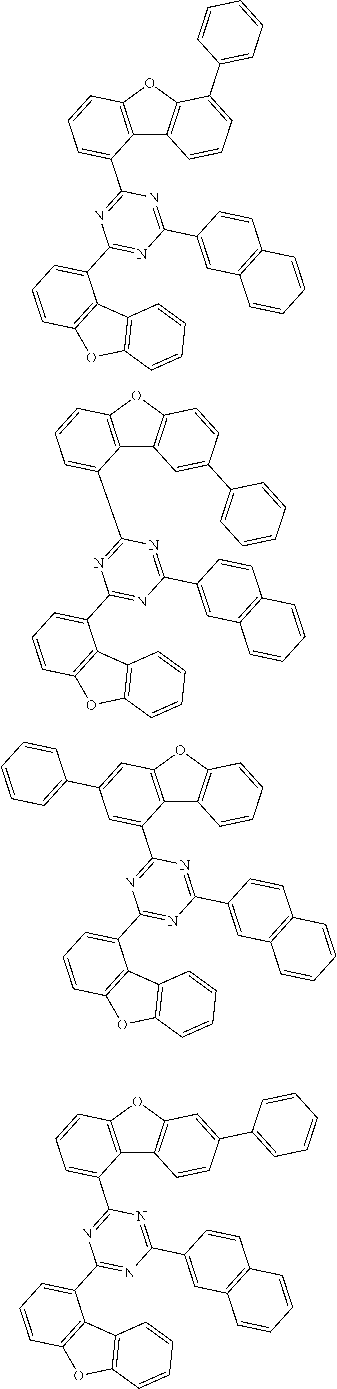

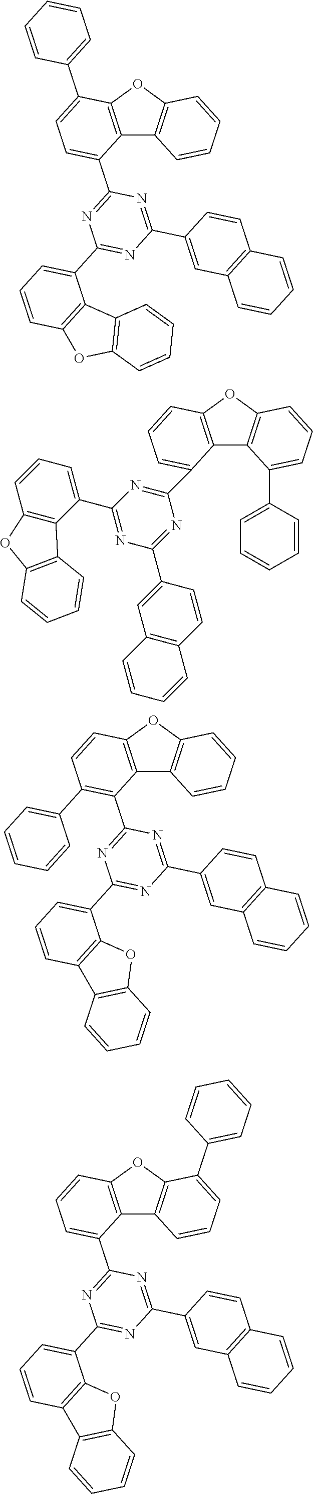

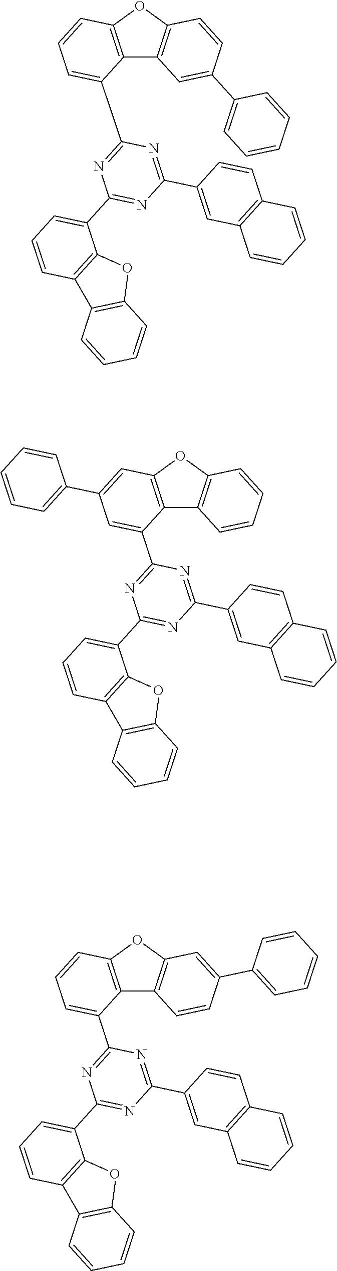 [SMILES: c1ccc(-c2ccc3oc4cccc(-c5nc(-c6ccc7ccccc7c6)nc(-c6cccc7c6oc6ccccc67)n5)c4c3c2)cc1 ; c1ccc(-c2cc(-c3nc(-c4ccc5ccccc5c4)nc(-c4cccc5c4oc4ccccc45)n3)c3c(c2)oc2ccccc23)cc1 ; c1ccc(-c2ccc3c(c2)oc2cccc(-c4nc(-c5ccc6ccccc6c5)nc(-c5cccc6c5oc5ccccc56)n4)c23)cc1]

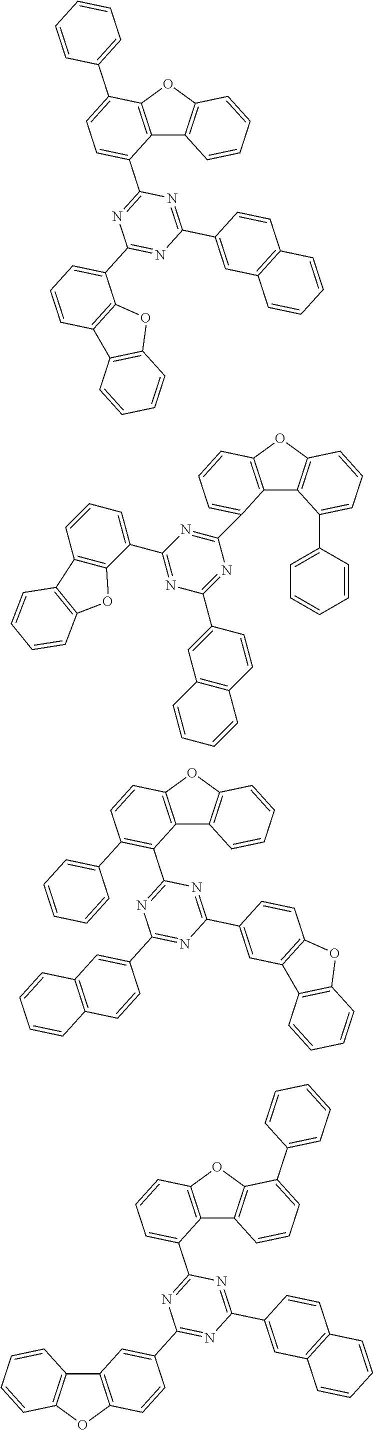

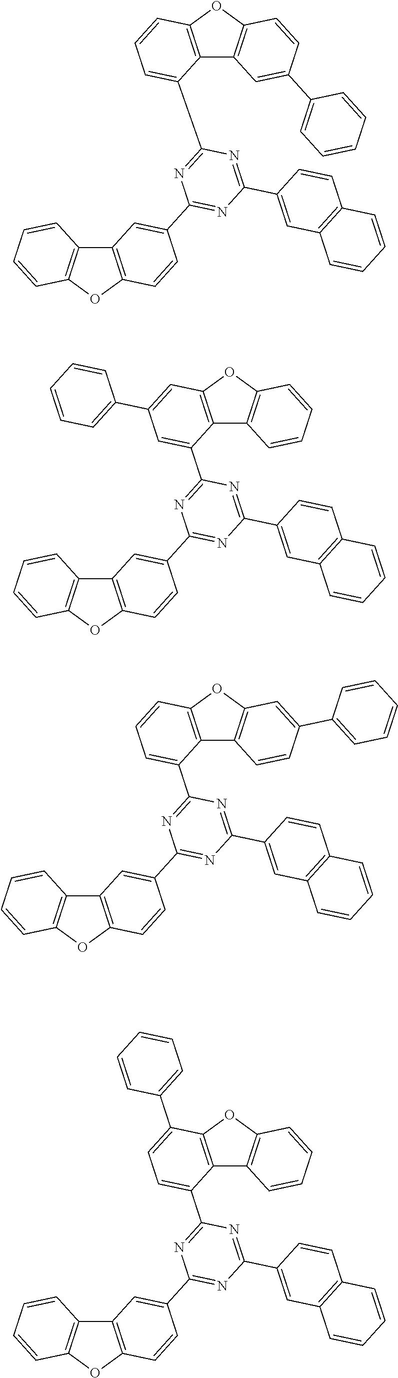 [SMILES: c1ccc(-c2ccc3oc4cccc(-c5nc(-c6ccc7ccccc7c6)nc(-c6ccc7oc8ccccc8c7c6)n5)c4c3c2)cc1 ; c1ccc(-c2cc(-c3nc(-c4ccc5ccccc5c4)nc(-c4ccc5oc6ccccc6c5c4)n3)c3c(c2)oc2ccccc23)cc1 ; c1ccc(-c2ccc3c(c2)oc2cccc(-c4nc(-c5ccc6ccccc6c5)nc(-c5ccc6oc7ccccc7c6c5)n4)c23)cc1 ; c1ccc(-c2ccc(-c3nc(-c4ccc5ccccc5c4)nc(-c4ccc5oc6ccccc6c5c4)n3)c3c2oc2ccccc23)cc1]

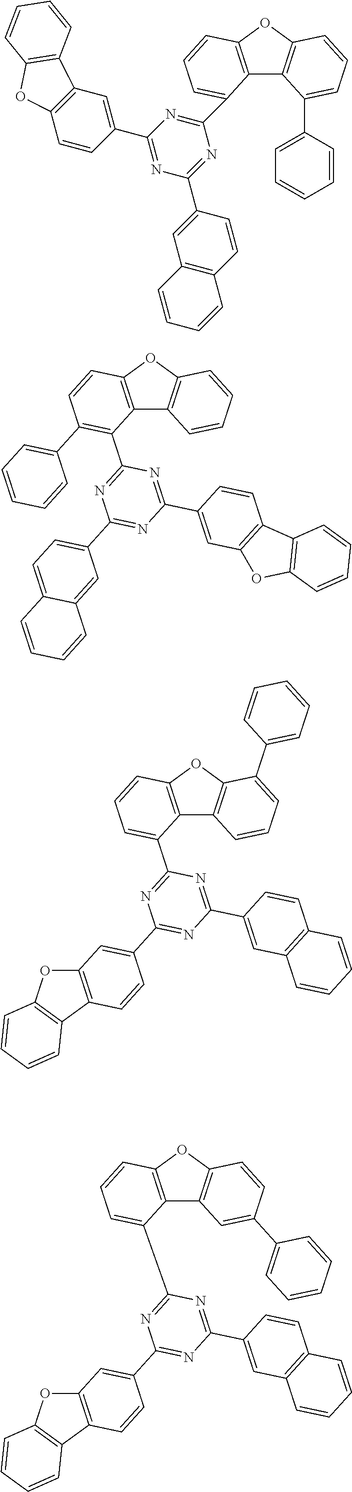

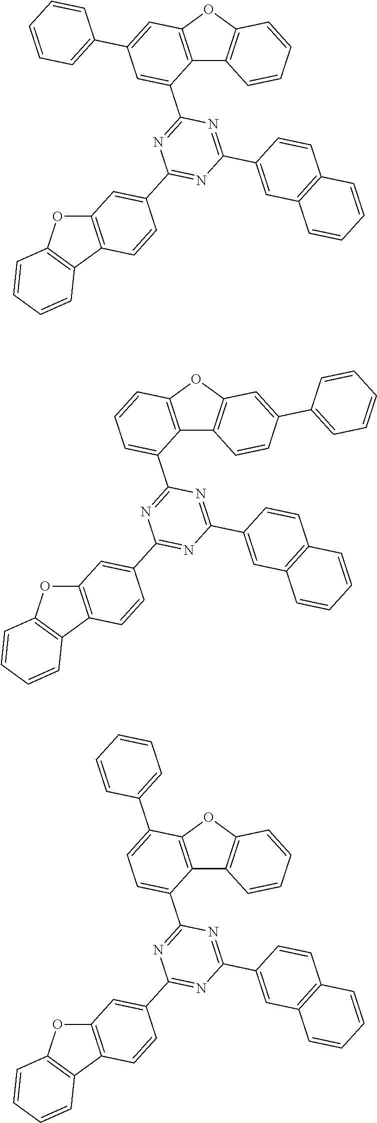 [SMILES: c1ccc(-c2cc(-c3nc(-c4ccc5ccccc5c4)nc(-c4ccc5c(c4)oc4ccccc45)n3)c3c(c2)oc2ccccc23)cc1 ; c1ccc(-c2ccc3c(c2)oc2cccc(-c4nc(-c5ccc6ccccc6c5)nc(-c5ccc6c(c5)oc5ccccc56)n4)c23)cc1 ; c1ccc(-c2ccc(-c3nc(-c4ccc5ccccc5c4)nc(-c4ccc5c(c4)oc4ccccc45)n3)c3c2oc2ccccc23)cc1]

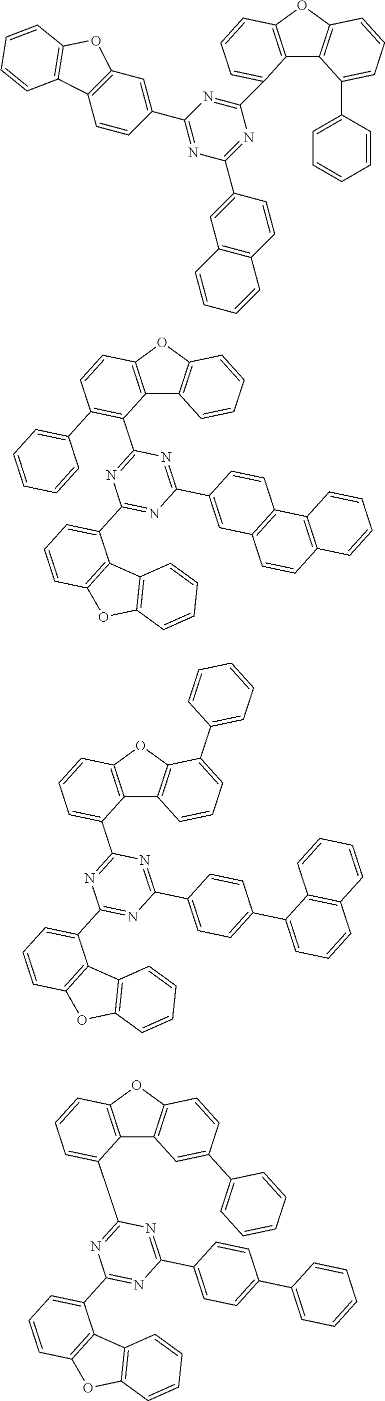

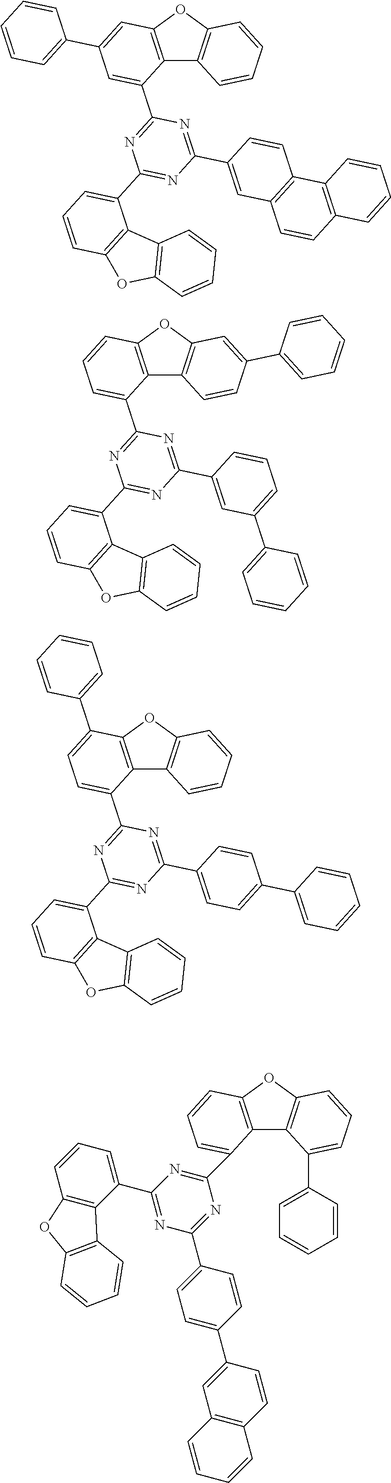

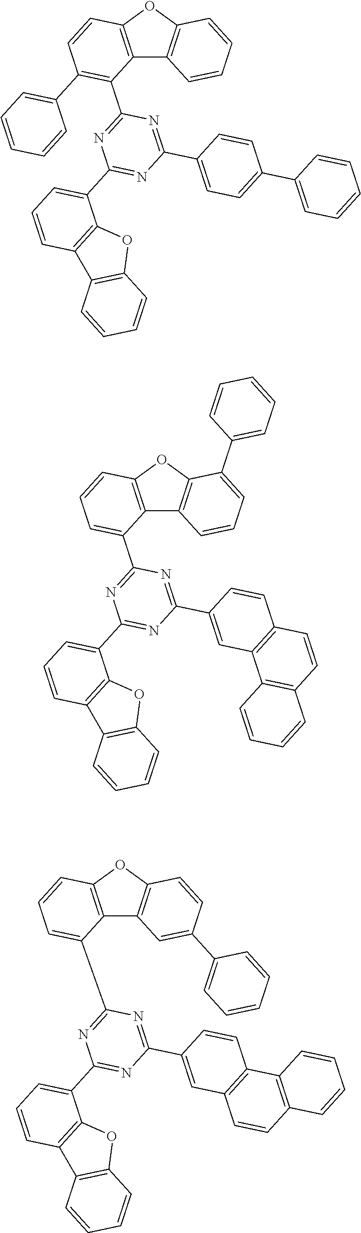 [SMILES: c1ccc(-c2ccc(-c3nc(-c4cccc5c4oc4ccccc45)nc(-c4c(-c5ccccc5)ccc5oc6ccccc6c45)n3)cc2)cc1 ; c1ccc(-c2cccc3c2oc2cccc(-c4nc(-c5ccc6ccc7ccccc7c6c5)nc(-c5cccc6c5oc5ccccc56)n4)c23)cc1 ; c1ccc(-c2ccc3oc4cccc(-c5nc(-c6ccc7c(ccc8ccccc87)c6)nc(-c6cccc7c6oc6ccccc67)n5)c4c3c2)cc1]

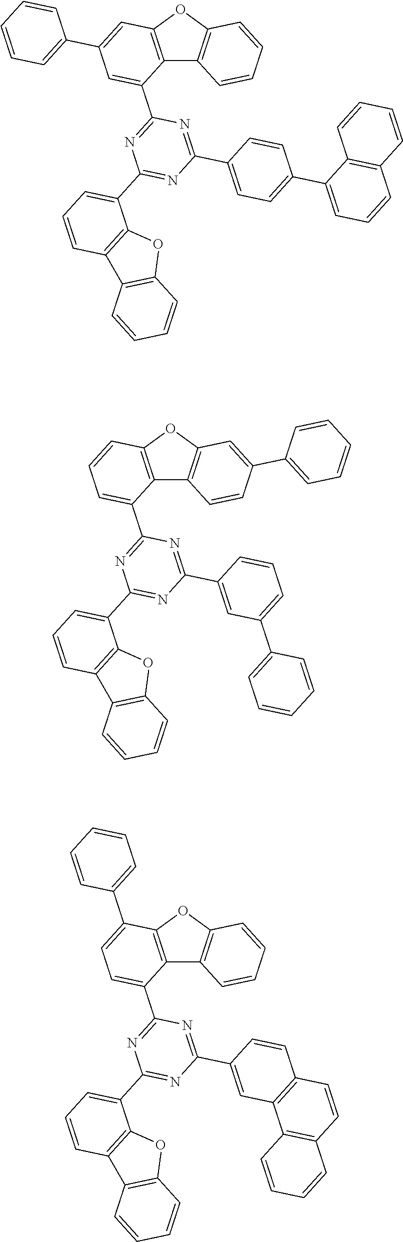 [SMILES: c1ccc(-c2cc(-c3nc(-c4ccc(-c5cccc6ccccc56)cc4)nc(-c4cccc5c4oc4ccccc45)n3)c3c(c2)oc2ccccc23)cc1 ; c1ccc(-c2cccc(-c3nc(-c4cccc5c4oc4ccccc45)nc(-c4cccc5oc6cc(-c7ccccc7)ccc6c45)n3)c2)cc1 ; c1ccc(-c2ccc(-c3nc(-c4ccc5ccc6ccccc6c5c4)nc(-c4cccc5c4oc4ccccc45)n3)c3c2oc2ccccc23)cc1]

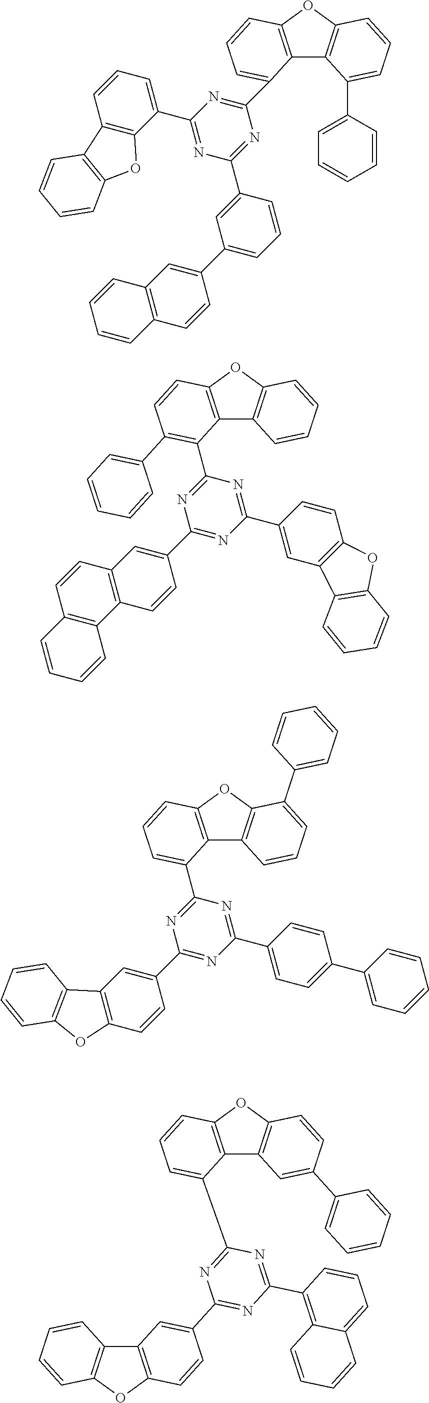 [SMILES: c1ccc(-c2cccc3oc4cccc(-c5nc(-c6cccc(-c7ccc8ccccc8c7)c6)nc(-c6cccc7c6oc6ccccc67)n5)c4c23)cc1 ; c1ccc(-c2ccc3oc4ccccc4c3c2-c2nc(-c3ccc4c(ccc5ccccc54)c3)nc(-c3ccc4oc5ccccc5c4c3)n2)cc1 ; c1ccc(-c2ccc(-c3nc(-c4ccc5oc6ccccc6c5c4)nc(-c4cccc5oc6c(-c7ccccc7)cccc6c45)n3)cc2)cc1 ; c1ccc(-c2ccc3oc4cccc(-c5nc(-c6ccc7oc8ccccc8c7c6)nc(-c6cccc7ccccc67)n5)c4c3c2)cc1]

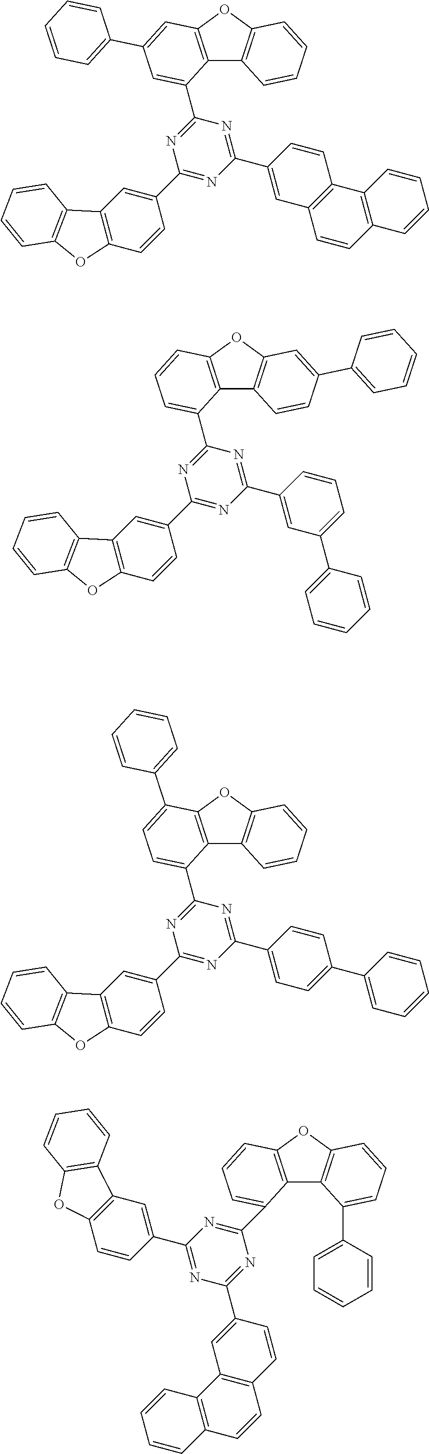 [SMILES: c1ccc(-c2cc(-c3nc(-c4ccc5c(ccc6ccccc65)c4)nc(-c4ccc5oc6ccccc6c5c4)n3)c3c(c2)oc2ccccc23)cc1 ; c1ccc(-c2cccc(-c3nc(-c4ccc5oc6ccccc6c5c4)nc(-c4cccc5oc6cc(-c7ccccc7)ccc6c45)n3)c2)cc1 ; c1ccc(-c2ccc(-c3nc(-c4ccc5oc6ccccc6c5c4)nc(-c4ccc(-c5ccccc5)c5oc6ccccc6c45)n3)cc2)cc1 ; c1ccc(-c2cccc3oc4cccc(-c5nc(-c6ccc7ccc8ccccc8c7c6)nc(-c6ccc7oc8ccccc8c7c6)n5)c4c23)cc1]

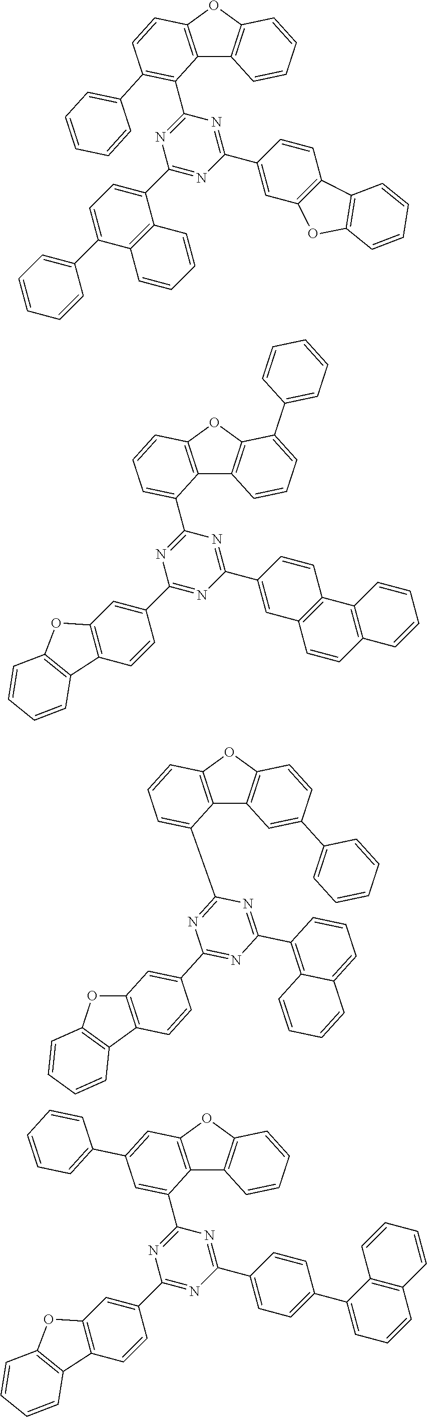

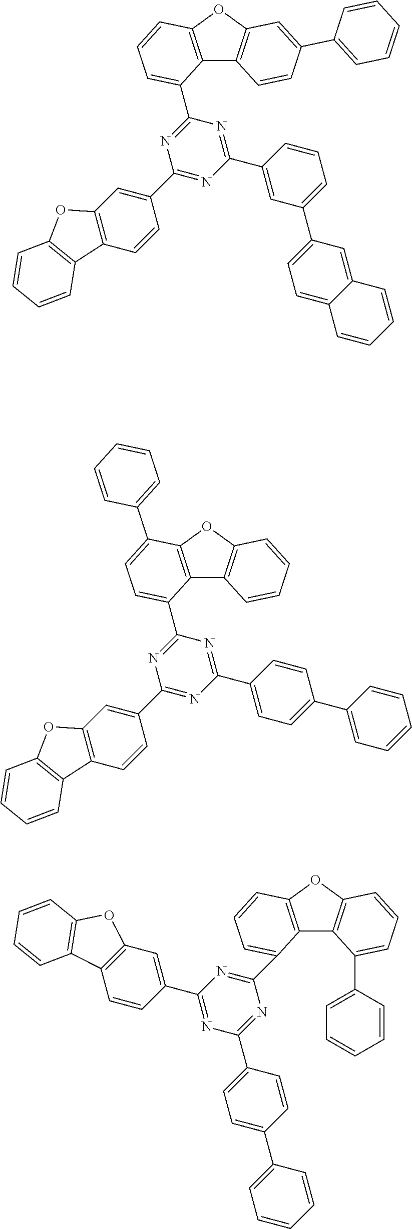 [SMILES: c1ccc(-c2ccc3c(c2)oc2cccc(-c4nc(-c5cccc(-c6ccc7ccccc7c6)c5)nc(-c5ccc6c(c5)oc5ccccc56)n4)c23)cc1 ; c1ccc(-c2ccc(-c3nc(-c4ccc5c(c4)oc4ccccc45)nc(-c4ccc(-c5ccccc5)c5oc6ccccc6c45)n3)cc2)cc1 ; c1ccc(-c2ccc(-c3nc(-c4ccc5c(c4)oc4ccccc45)nc(-c4cccc5oc6cccc(-c7ccccc7)c6c45)n3)cc2)cc1]

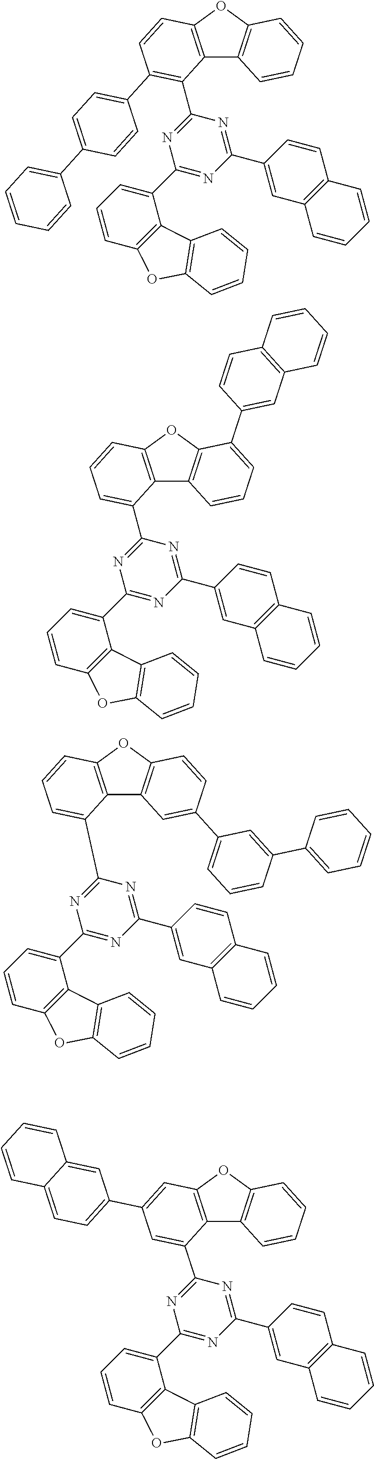

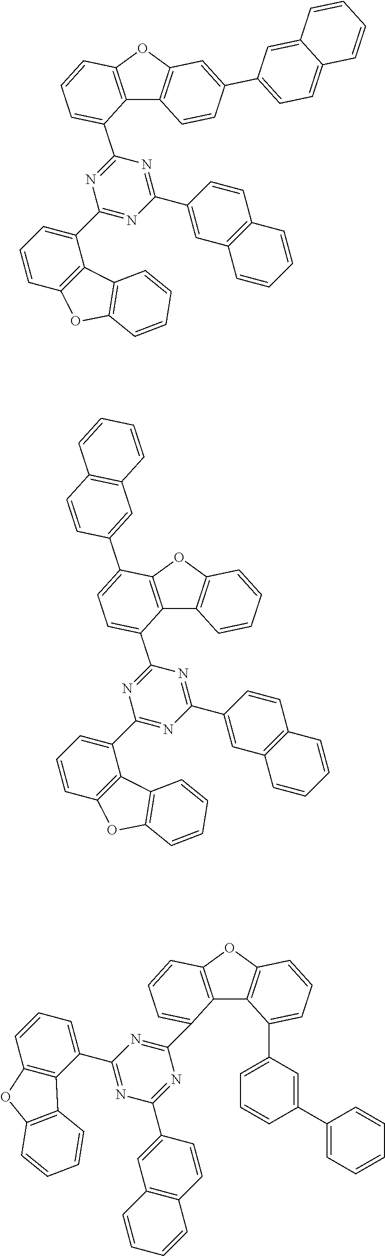 [SMILES: c1ccc2cc(-c3ccc4c(c3)oc3cccc(-c5nc(-c6ccc7ccccc7c6)nc(-c6cccc7oc8ccccc8c67)n5)c34)ccc2c1 ; c1ccc2cc(-c3nc(-c4cccc5oc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8oc9ccccc9c78)n6)c5c34)c2)cc1]

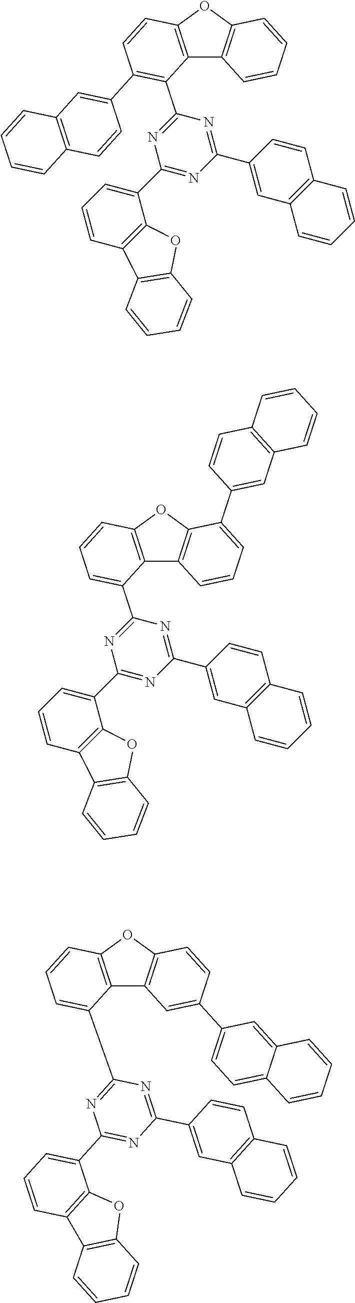 [SMILES: c1ccc2cc(-c3nc(-c4cccc5c4oc4ccccc45)nc(-c4c(-c5ccc6ccccc6c5)ccc5oc6ccccc6c45)n3)ccc2c1 ; c1ccc2cc(-c3nc(-c4cccc5c4oc4ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cccc8c7oc7ccccc78)n6)c5c4c3)ccc2c1]

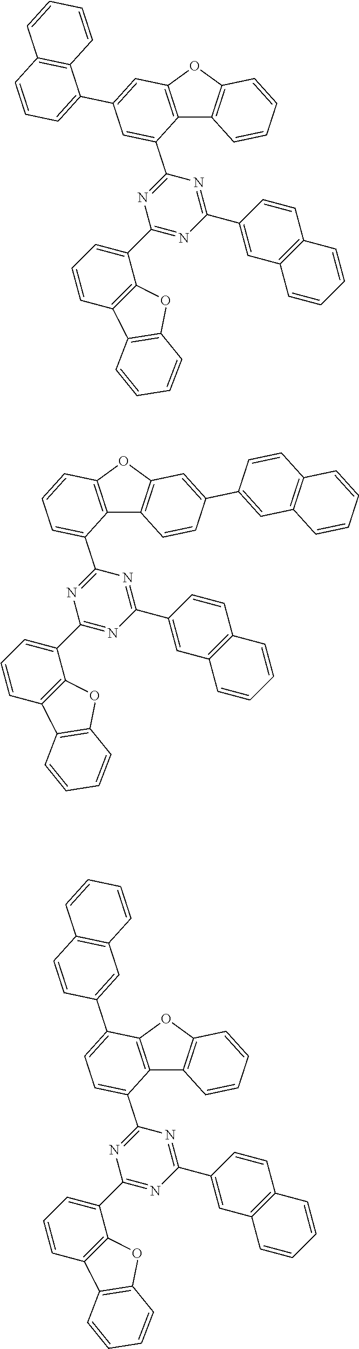 [SMILES: c1ccc2cc(-c3nc(-c4cccc5c4oc4ccccc45)nc(-c4cc(-c5cccc6ccccc56)cc5oc6ccccc6c45)n3)ccc2c1 ; c1ccc2cc(-c3ccc4c(c3)oc3cccc(-c5nc(-c6ccc7ccccc7c6)nc(-c6cccc7c6oc6ccccc67)n5)c34)ccc2c1 ; c1ccc2cc(-c3nc(-c4cccc5c4oc4ccccc45)nc(-c4ccc(-c5ccc6ccccc6c5)c5oc6ccccc6c45)n3)ccc2c1]

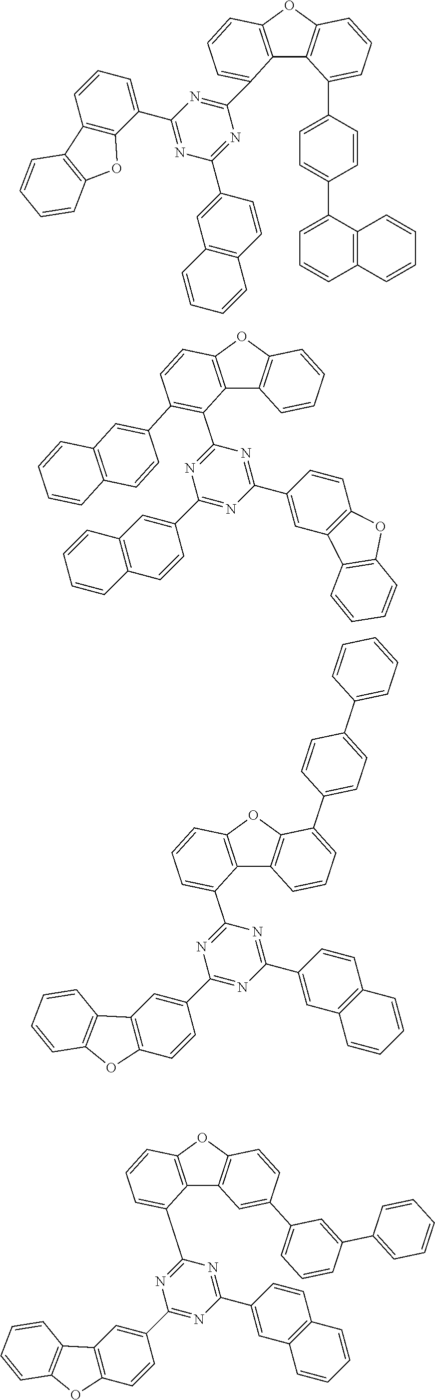

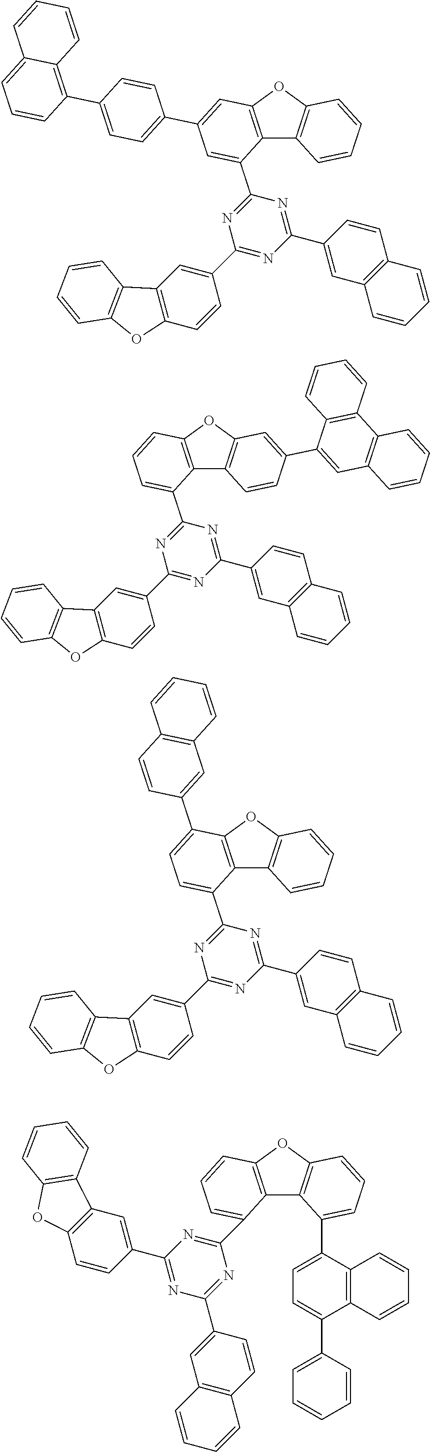 [SMILES: c1ccc2cc(-c3nc(-c4ccc5oc6ccccc6c5c4)nc(-c4cc(-c5ccc(-c6cccc7ccccc67)cc5)cc5oc6ccccc6c45)n3)ccc2c1 ; c1ccc2cc(-c3nc(-c4ccc5oc6ccccc6c5c4)nc(-c4cccc5oc6cc(-c7cc8ccccc8c8ccccc78)ccc6c45)n3)ccc2c1 ; c1ccc2cc(-c3nc(-c4ccc5oc6ccccc6c5c4)nc(-c4ccc(-c5ccc6ccccc6c5)c5oc6ccccc6c45)n3)ccc2c1 ; c1ccc(-c2ccc(-c3cccc4oc5cccc(-c6nc(-c7ccc8ccccc8c7)nc(-c7ccc8oc9ccccc9c8c7)n6)c5c34)c3ccccc23)cc1]

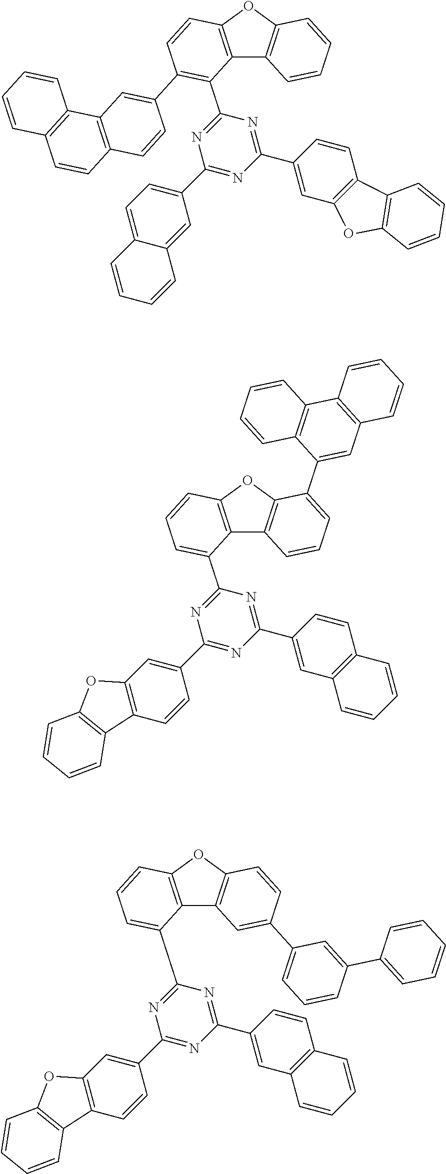 [SMILES: c1ccc2cc(-c3nc(-c4ccc5c(c4)oc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)oc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6nc(-c7ccc8ccccc8c7)nc(-c7ccc8c(c7)oc7ccccc78)n6)c5c4c3)c2)cc1]

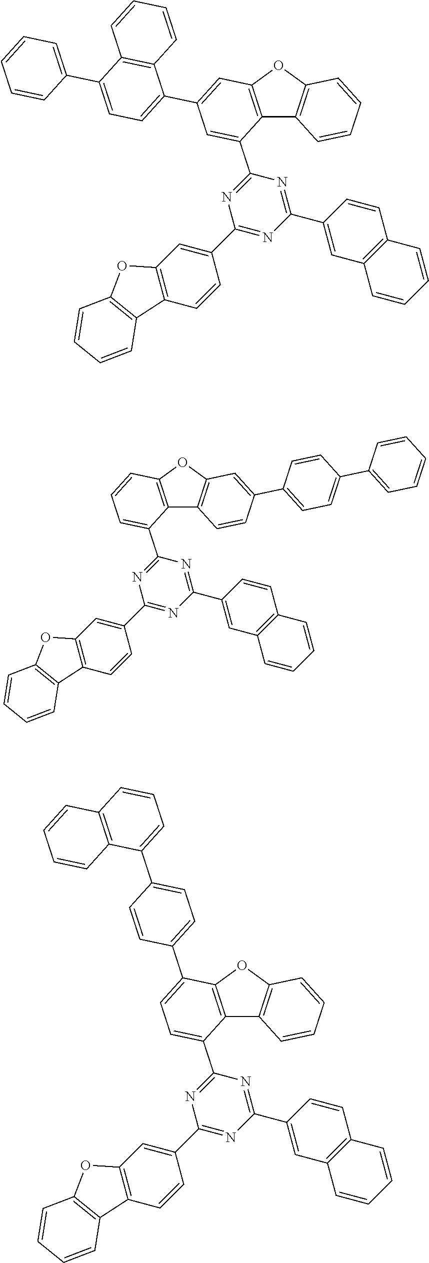 [SMILES: c1ccc(-c2ccc(-c3cc(-c4nc(-c5ccc6ccccc6c5)nc(-c5ccc6c(c5)oc5ccccc56)n4)c4c(c3)oc3ccccc34)c3ccccc23)cc1 ; c1ccc(-c2ccc(-c3ccc4c(c3)oc3cccc(-c5nc(-c6ccc7ccccc7c6)nc(-c6ccc7c(c6)oc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1]

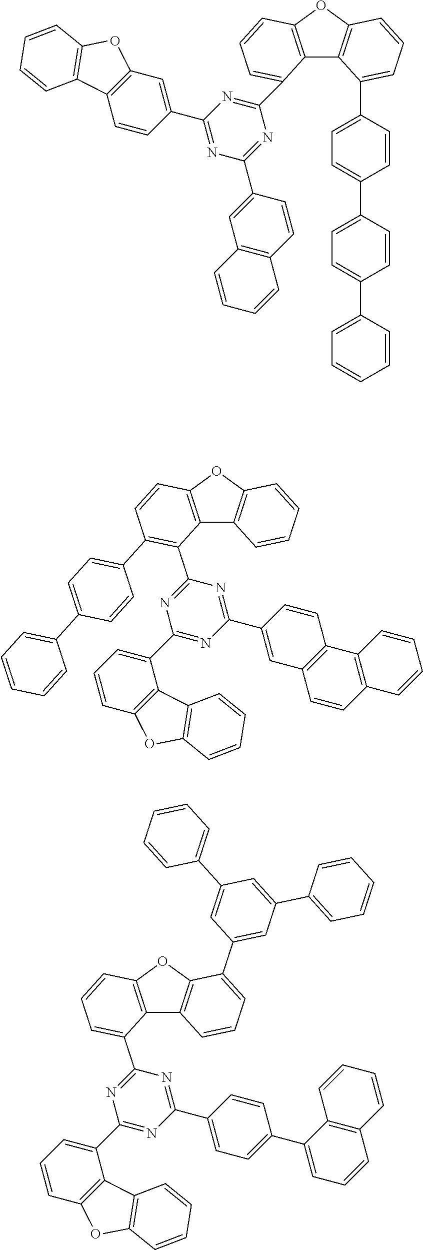 [SMILES: c1ccc(-c2ccc(-c3ccc(-c4cccc5oc6cccc(-c7nc(-c8ccc9ccccc9c8)nc(-c8ccc9c(c8)oc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5oc6ccccc6c45)n3)cc2)cc1 ; c1ccc(-c2cc(-c3ccccc3)cc(-c3cccc4c3oc3cccc(-c5nc(-c6ccc(-c7cccc8ccccc78)cc6)nc(-c6cccc7oc8ccccc8c67)n5)c34)c2)cc1]

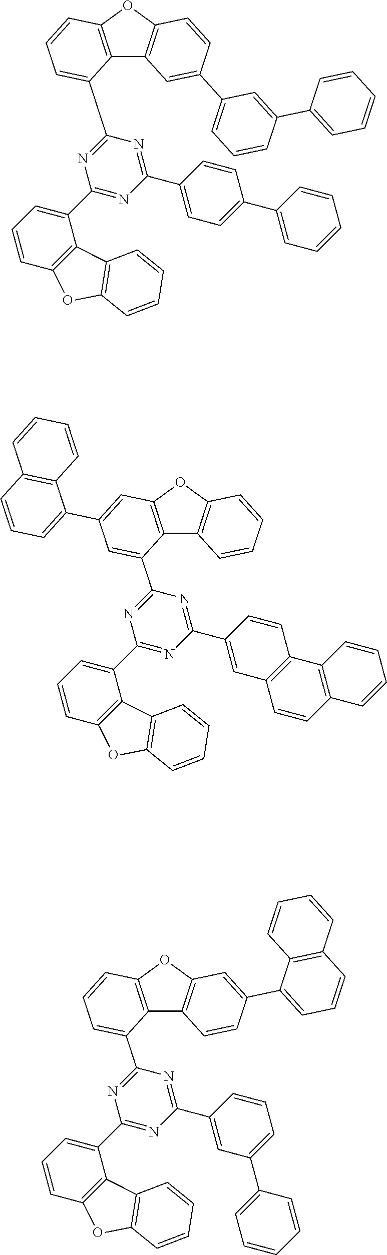 [SMILES: c1ccc(-c2ccc(-c3nc(-c4cccc5oc6ccccc6c45)nc(-c4cccc5oc6ccc(-c7cccc(-c8ccccc8)c7)cc6c45)n3)cc2)cc1 ; c1ccc2c(-c3cc(-c4nc(-c5ccc6c(ccc7ccccc76)c5)nc(-c5cccc6oc7ccccc7c56)n4)c4c(c3)oc3ccccc34)cccc2c1 ; c1ccc(-c2cccc(-c3nc(-c4cccc5oc6ccccc6c45)nc(-c4cccc5oc6cc(-c7cccc8ccccc78)ccc6c45)n3)c2)cc1]

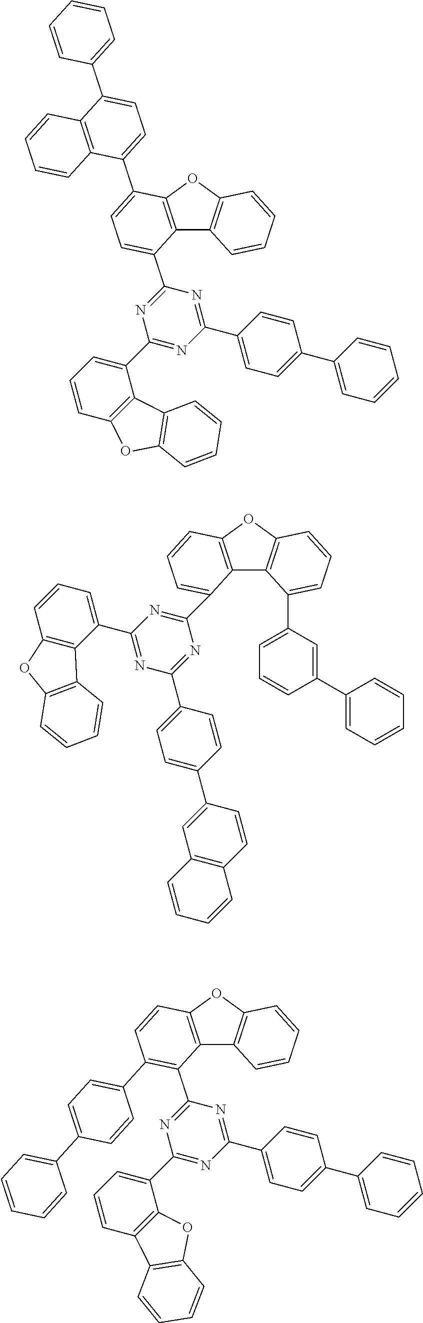 [SMILES: c1ccc(-c2ccc(-c3nc(-c4cccc5oc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8oc9ccccc9c78)n6)c5c34)c2)cc1 ; c1ccc(-c2ccc(-c3nc(-c4cccc5c4oc4ccccc45)nc(-c4c(-c5ccc(-c6ccccc6)cc5)ccc5oc6ccccc6c45)n3)cc2)cc1]

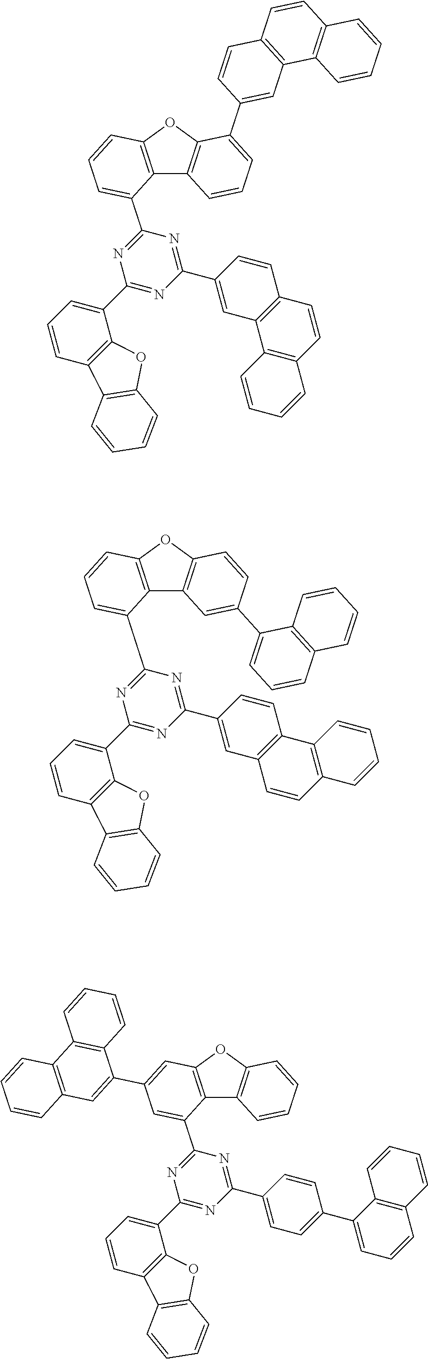 [SMILES: c1ccc2c(c1)ccc1ccc(-c3nc(-c4cccc5c4oc4ccccc45)nc(-c4cccc5oc6c(-c7ccc8ccc9ccccc9c8c7)cccc6c45)n3)cc12 ; c1ccc2c(-c3ccc4oc5cccc(-c6nc(-c7ccc8c(ccc9ccccc98)c7)nc(-c7cccc8c7oc7ccccc78)n6)c5c4c3)cccc2c1 ; c1ccc2c(-c3ccc(-c4nc(-c5cccc6c5oc5ccccc56)nc(-c5cc(-c6cc7ccccc7c7ccccc67)cc6oc7ccccc7c56)n4)cc3)cccc2c1]

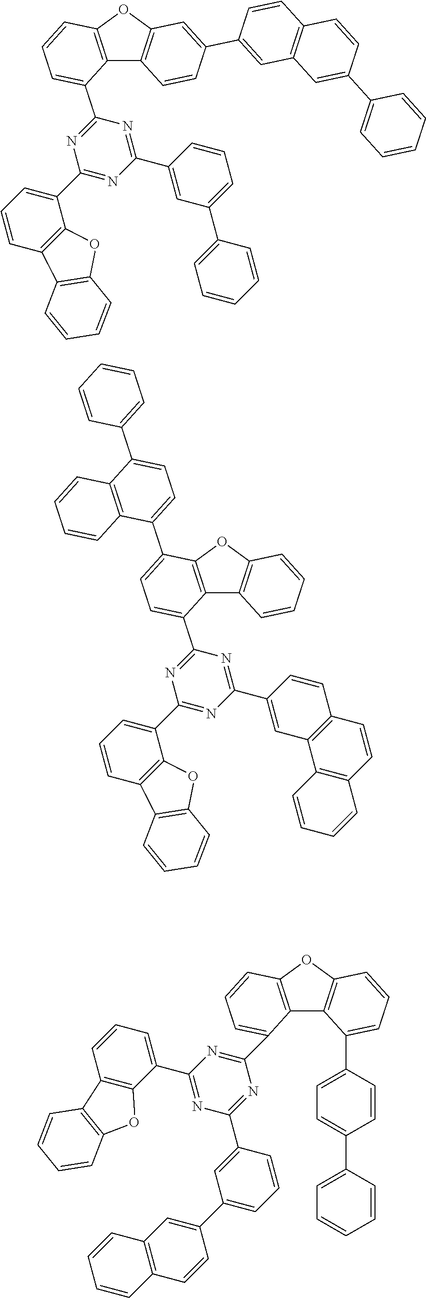

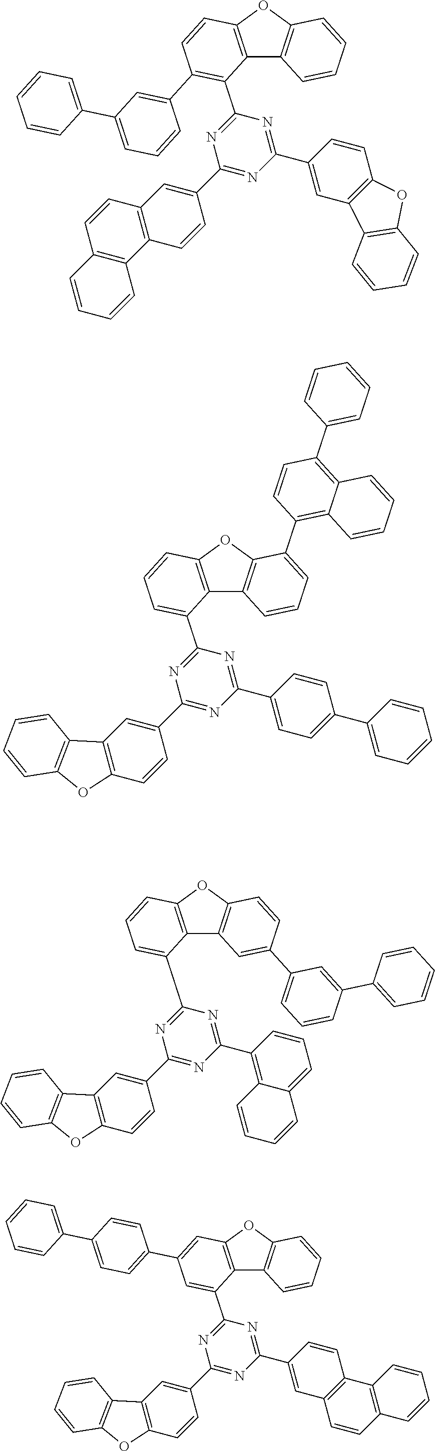 [SMILES: c1ccc(-c2cccc(-c3ccc4oc5ccccc5c4c3-c3nc(-c4ccc5c(ccc6ccccc65)c4)nc(-c4ccc5oc6ccccc6c5c4)n3)c2)cc1 ; c1ccc(-c2ccc(-c3nc(-c4ccc5oc6ccccc6c5c4)nc(-c4cccc5oc6c(-c7ccc(-c8ccccc8)c8ccccc78)cccc6c45)n3)cc2)cc1 ; c1ccc(-c2cccc(-c3ccc4oc5cccc(-c6nc(-c7ccc8oc9ccccc9c8c7)nc(-c7cccc8ccccc78)n6)c5c4c3)c2)cc1 ; c1ccc(-c2ccc(-c3cc(-c4nc(-c5ccc6c(ccc7ccccc76)c5)nc(-c5ccc6oc7ccccc7c6c5)n4)c4c(c3)oc3ccccc34)cc2)cc1]

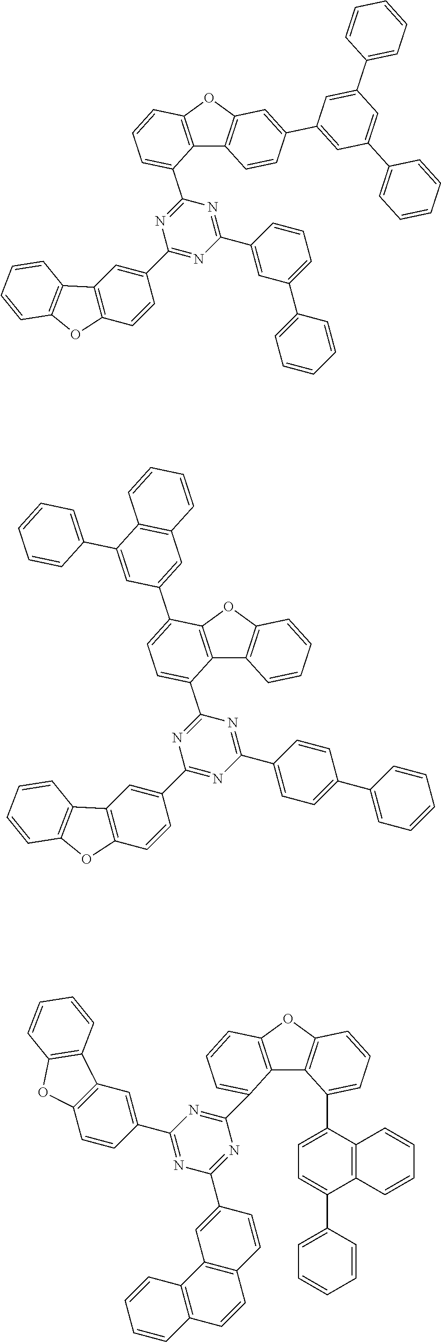 [SMILES: c1ccc(-c2cc(-c3ccccc3)cc(-c3ccc4c(c3)oc3cccc(-c5nc(-c6cccc(-c7ccccc7)c6)nc(-c6ccc7oc8ccccc8c7c6)n5)c34)c2)cc1 ; c1ccc(-c2ccc(-c3nc(-c4ccc5oc6ccccc6c5c4)nc(-c4ccc(-c5cc(-c6ccccc6)c6ccccc6c5)c5oc6ccccc6c45)n3)cc2)cc1 ; c1ccc(-c2ccc(-c3cccc4oc5cccc(-c6nc(-c7ccc8ccc9ccccc9c8c7)nc(-c7ccc8oc9ccccc9c8c7)n6)c5c34)c3ccccc23)cc1]

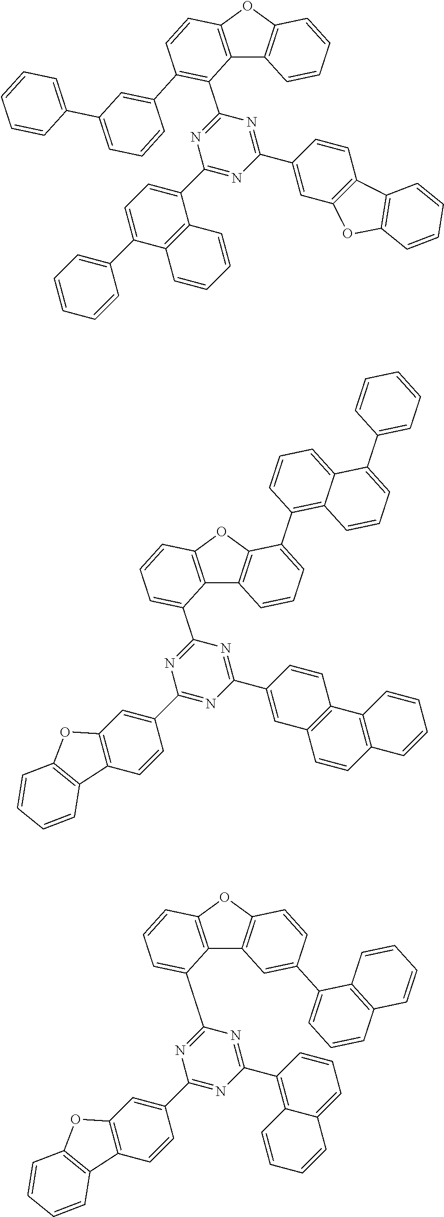 [SMILES: c1ccc(-c2cccc(-c3ccc4oc5ccccc5c4c3-c3nc(-c4ccc5c(c4)oc4ccccc45)nc(-c4ccc(-c5ccccc5)c5ccccc45)n3)c2)cc1 ; c1ccc(-c2cccc3c(-c4cccc5c4oc4cccc(-c6nc(-c7ccc8c(ccc9ccccc98)c7)nc(-c7ccc8c(c7)oc7ccccc78)n6)c45)cccc23)cc1 ; c1ccc2c(-c3ccc4oc5cccc(-c6nc(-c7ccc8c(c7)oc7ccccc78)nc(-c7cccc8ccccc78)n6)c5c4c3)cccc2c1]

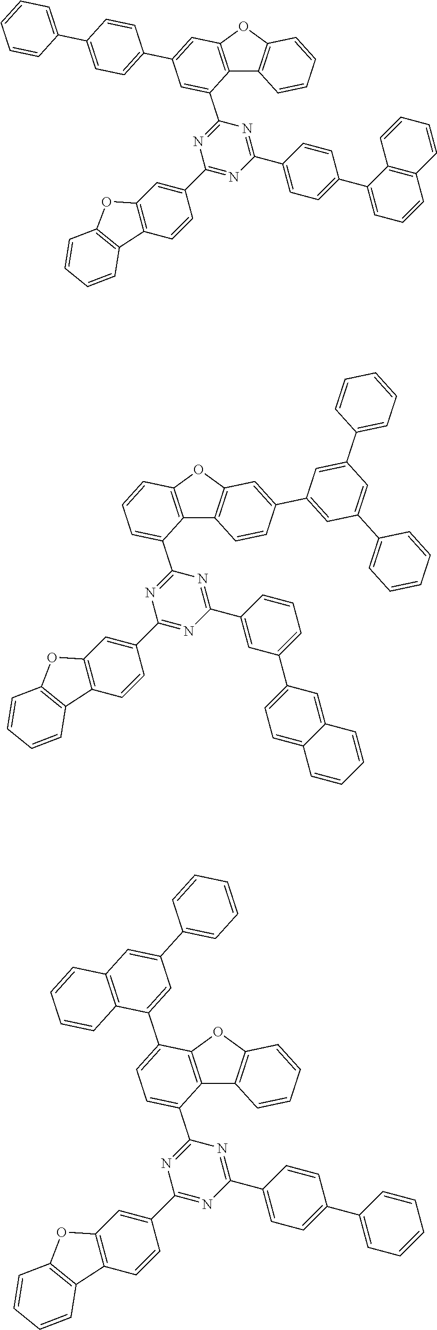 [SMILES: c1ccc(-c2ccc(-c3cc(-c4nc(-c5ccc(-c6cccc7ccccc67)cc5)nc(-c5ccc6c(c5)oc5ccccc56)n4)c4c(c3)oc3ccccc34)cc2)cc1 ; c1ccc(-c2cc(-c3ccccc3)cc(-c3ccc4c(c3)oc3cccc(-c5nc(-c6cccc(-c7ccc8ccccc8c7)c6)nc(-c6ccc7c(c6)oc6ccccc67)n5)c34)c2)cc1 ; c1ccc(-c2ccc(-c3nc(-c4ccc5c(c4)oc4ccccc45)nc(-c4ccc(-c5cc(-c6ccccc6)cc6ccccc56)c5oc6ccccc6c45)n3)cc2)cc1]

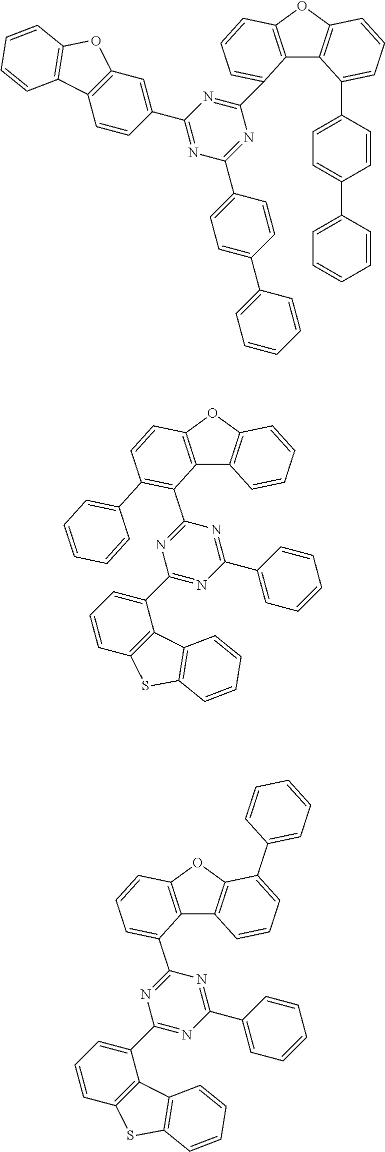 [SMILES: c1ccc(-c2ccc(-c3nc(-c4ccc5c(c4)oc4ccccc45)nc(-c4cccc5oc6cccc(-c7ccc(-c8ccccc8)cc7)c6c45)n3)cc2)cc1 ; c1ccc(-c2nc(-c3c(-c4ccccc4)ccc4oc5ccccc5c34)nc(-c3cccc4sc5ccccc5c34)n2)cc1 ; c1ccc(-c2nc(-c3cccc4oc5c(-c6ccccc6)cccc5c34)nc(-c3cccc4sc5ccccc5c34)n2)cc1]

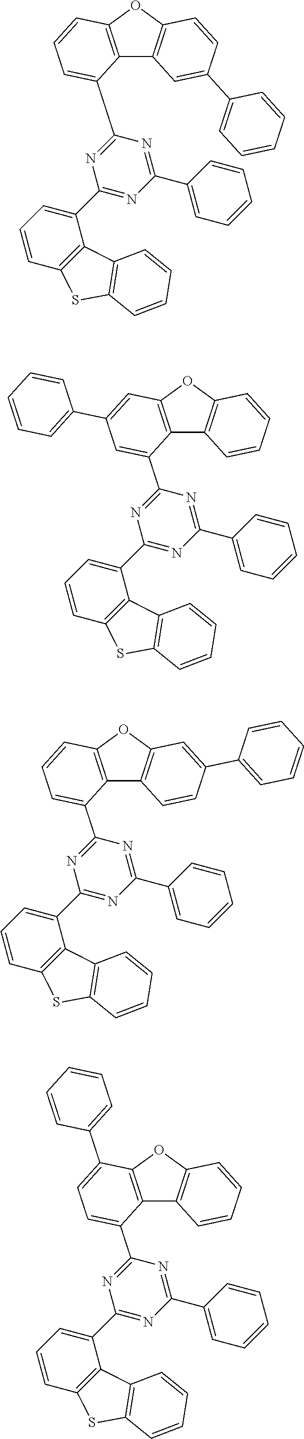 [SMILES: c1ccc(-c2ccc3oc4cccc(-c5nc(-c6ccccc6)nc(-c6cccc7sc8ccccc8c67)n5)c4c3c2)cc1 ; c1ccc(-c2cc(-c3nc(-c4ccccc4)nc(-c4cccc5sc6ccccc6c45)n3)c3c(c2)oc2ccccc23)cc1 ; c1ccc(-c2ccc3c(c2)oc2cccc(-c4nc(-c5ccccc5)nc(-c5cccc6sc7ccccc7c56)n4)c23)cc1 ; c1ccc(-c2nc(-c3cccc4sc5ccccc5c34)nc(-c3ccc(-c4ccccc4)c4oc5ccccc5c34)n2)cc1]

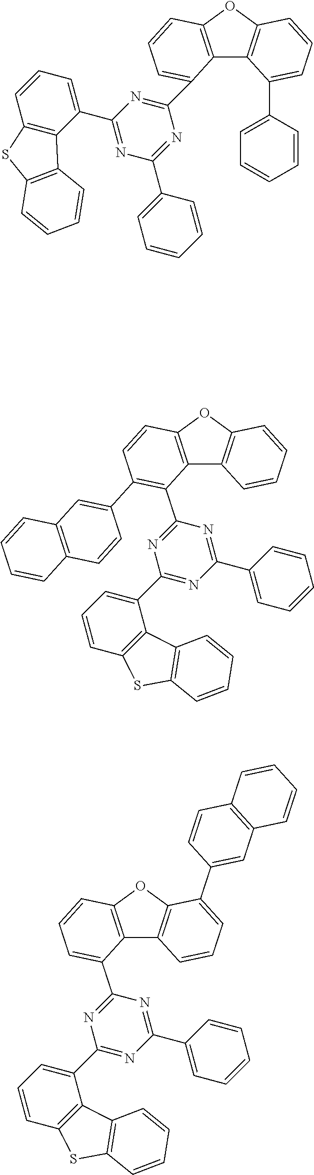 [SMILES: c1ccc(-c2nc(-c3cccc4sc5ccccc5c34)nc(-c3cccc4oc5cccc(-c6ccccc6)c5c34)n2)cc1 ; c1ccc(-c2nc(-c3c(-c4ccc5ccccc5c4)ccc4oc5ccccc5c34)nc(-c3cccc4sc5ccccc5c34)n2)cc1 ; c1ccc(-c2nc(-c3cccc4oc5c(-c6ccc7ccccc7c6)cccc5c34)nc(-c3cccc4sc5ccccc5c34)n2)cc1]

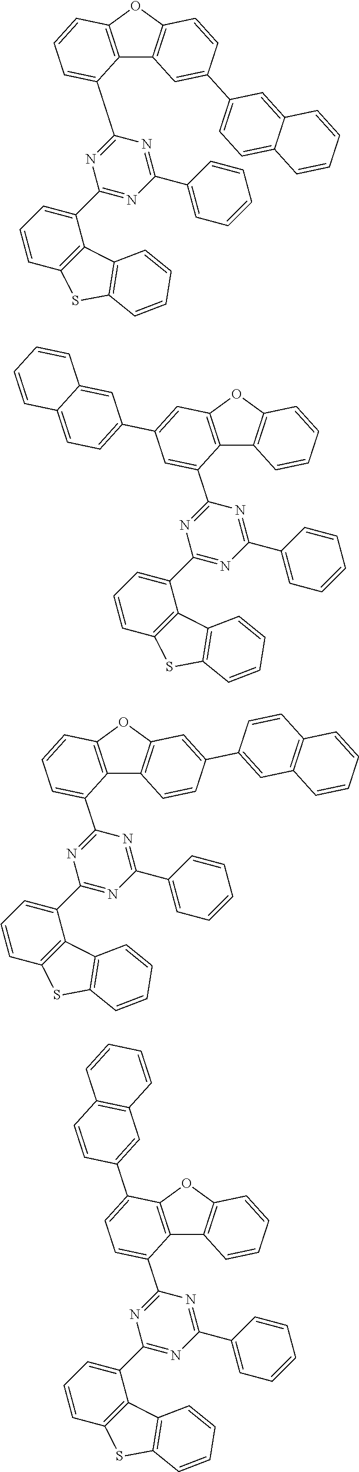

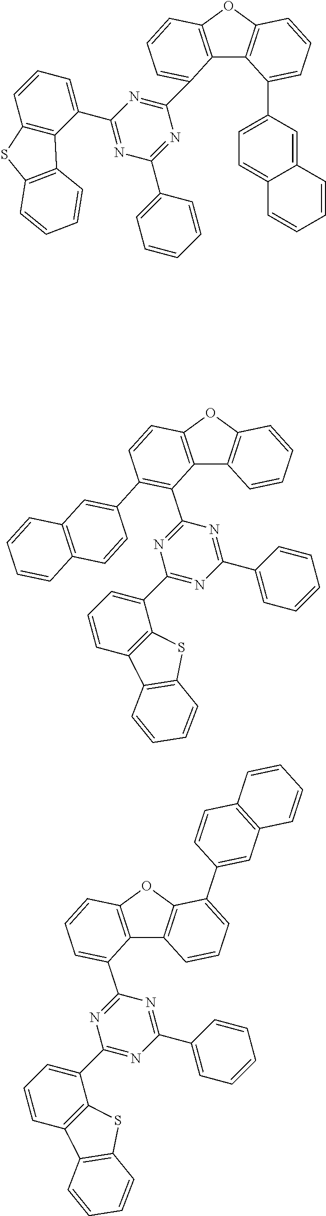 [SMILES: c1ccc(-c2nc(-c3cccc4sc5ccccc5c34)nc(-c3cccc4oc5cccc(-c6ccc7ccccc7c6)c5c34)n2)cc1 ; c1ccc(-c2nc(-c3cccc4c3sc3ccccc34)nc(-c3c(-c4ccc5ccccc5c4)ccc4oc5ccccc5c34)n2)cc1 ; c1ccc(-c2nc(-c3cccc4c3sc3ccccc34)nc(-c3cccc4oc5c(-c6ccc7ccccc7c6)cccc5c34)n2)cc1]

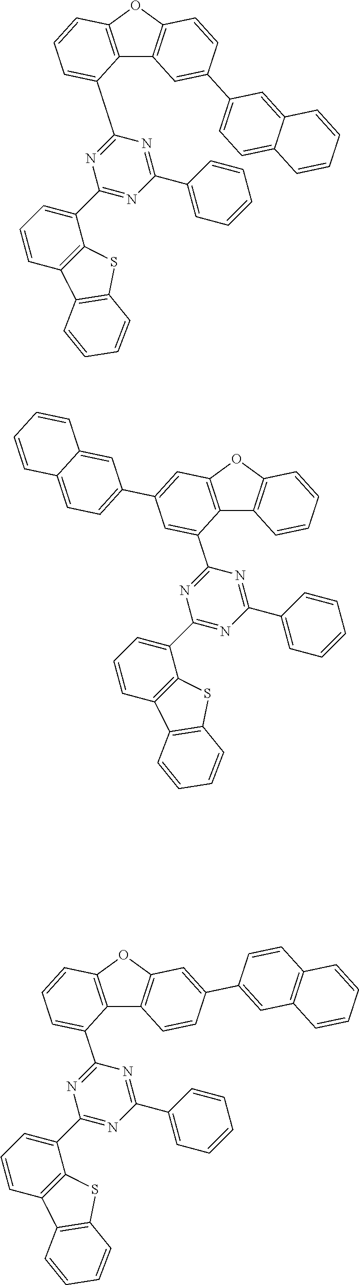 [SMILES: c1ccc(-c2nc(-c3cccc4c3sc3ccccc34)nc(-c3cccc4oc5ccc(-c6ccc7ccccc7c6)cc5c34)n2)cc1 ; c1ccc(-c2nc(-c3cccc4c3sc3ccccc34)nc(-c3cc(-c4ccc5ccccc5c4)cc4oc5ccccc5c34)n2)cc1 ; c1ccc(-c2nc(-c3cccc4c3sc3ccccc34)nc(-c3cccc4oc5cc(-c6ccc7ccccc7c6)ccc5c34)n2)cc1]

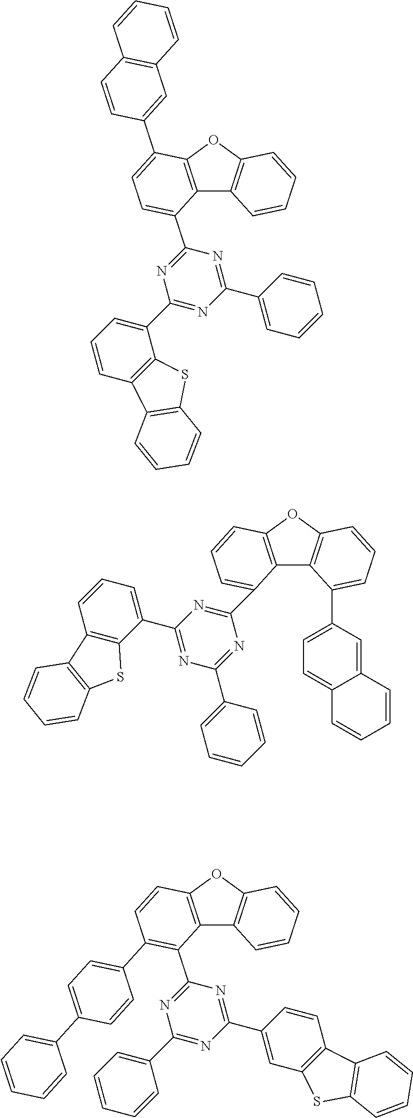 [SMILES: c1ccc(-c2nc(-c3cccc4c3sc3ccccc34)nc(-c3ccc(-c4ccc5ccccc5c4)c4oc5ccccc5c34)n2)cc1 ; c1ccc(-c2nc(-c3cccc4c3sc3ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc5c(c4)sc4ccccc45)n3)cc2)cc1]

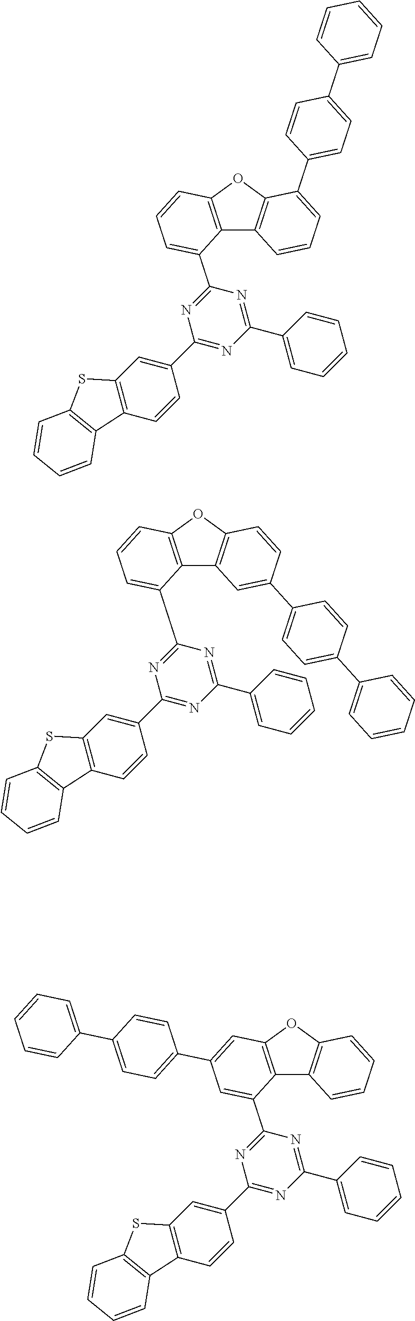 [SMILES: c1ccc(-c2ccc(-c3cccc4c3oc3cccc(-c5nc(-c6ccccc6)nc(-c6ccc7c(c6)sc6ccccc67)n5)c34)cc2)cc1 ; c1ccc(-c2ccc(-c3ccc4oc5cccc(-c6nc(-c7ccccc7)nc(-c7ccc8c(c7)sc7ccccc78)n6)c5c4c3)cc2)cc1 ; c1ccc(-c2ccc(-c3cc(-c4nc(-c5ccccc5)nc(-c5ccc6c(c5)sc5ccccc56)n4)c4c(c3)oc3ccccc34)cc2)cc1]

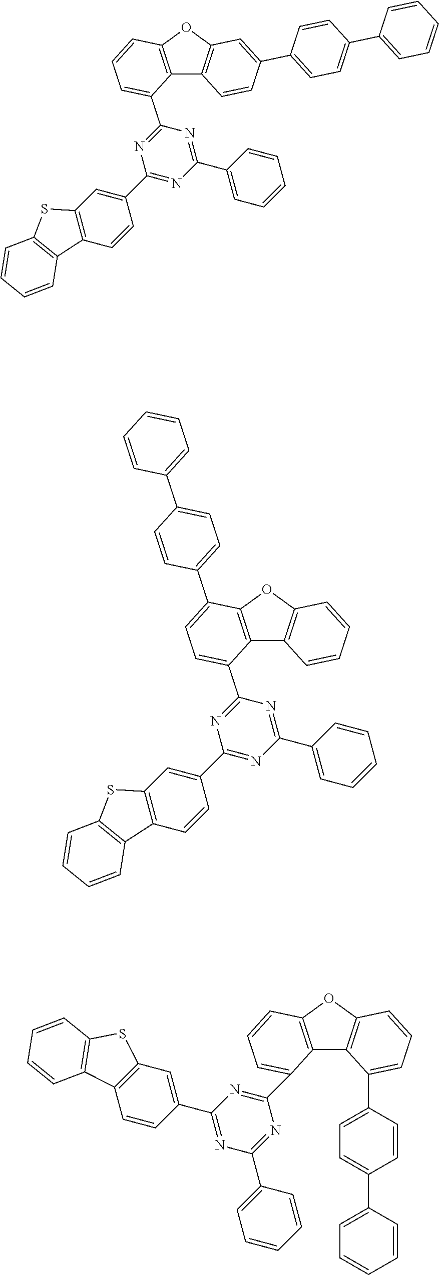 [SMILES: c1ccc(-c2ccc(-c3ccc4c(c3)oc3cccc(-c5nc(-c6ccccc6)nc(-c6ccc7c(c6)sc6ccccc67)n5)c34)cc2)cc1 ; c1ccc(-c2ccc(-c3ccc(-c4nc(-c5ccccc5)nc(-c5ccc6c(c5)sc5ccccc56)n4)c4c3oc3ccccc34)cc2)cc1 ; c1ccc(-c2ccc(-c3cccc4oc5cccc(-c6nc(-c7ccccc7)nc(-c7ccc8c(c7)sc7ccccc78)n6)c5c34)cc2)cc1]

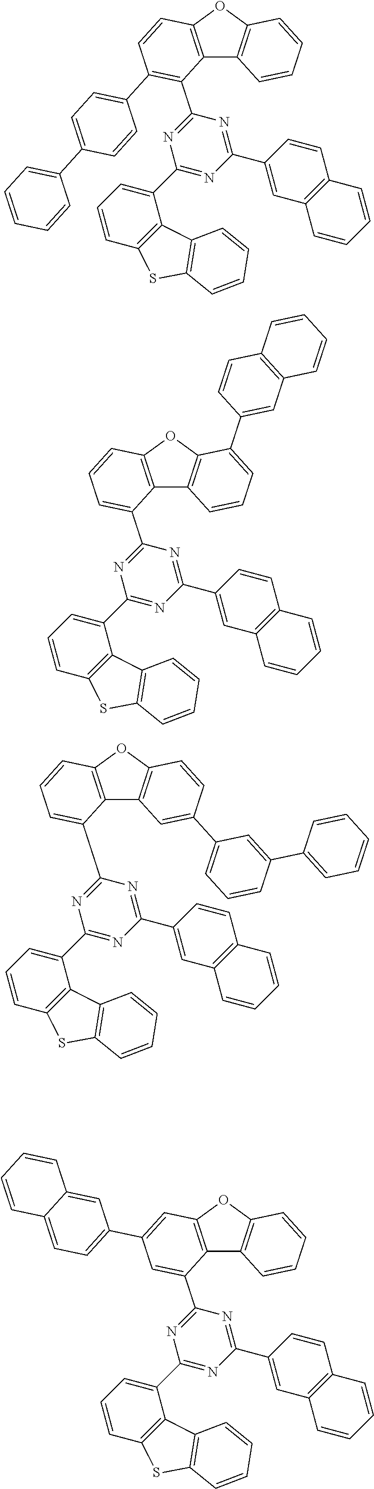

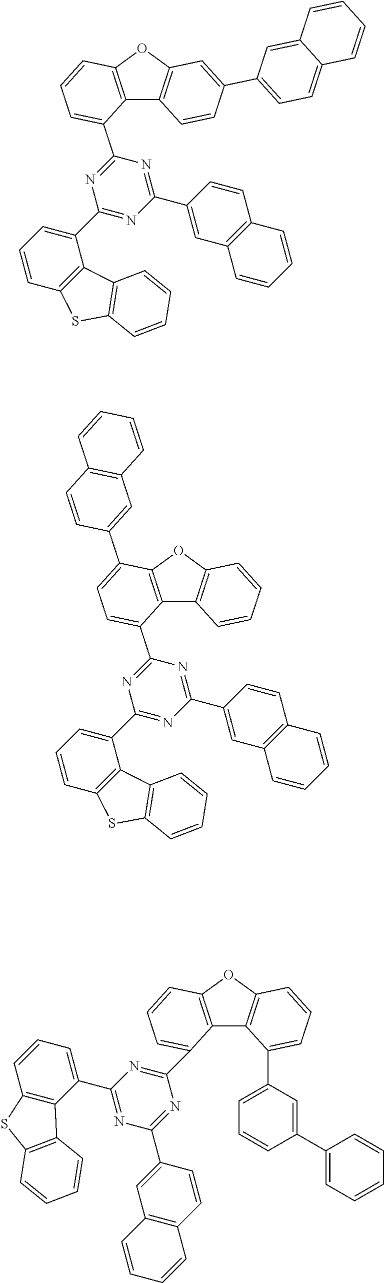 [SMILES: c1ccc2cc(-c3ccc4c(c3)oc3cccc(-c5nc(-c6ccc7ccccc7c6)nc(-c6cccc7sc8ccccc8c67)n5)c34)ccc2c1 ; c1ccc2cc(-c3nc(-c4cccc5sc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8sc9ccccc9c78)n6)c5c34)c2)cc1]

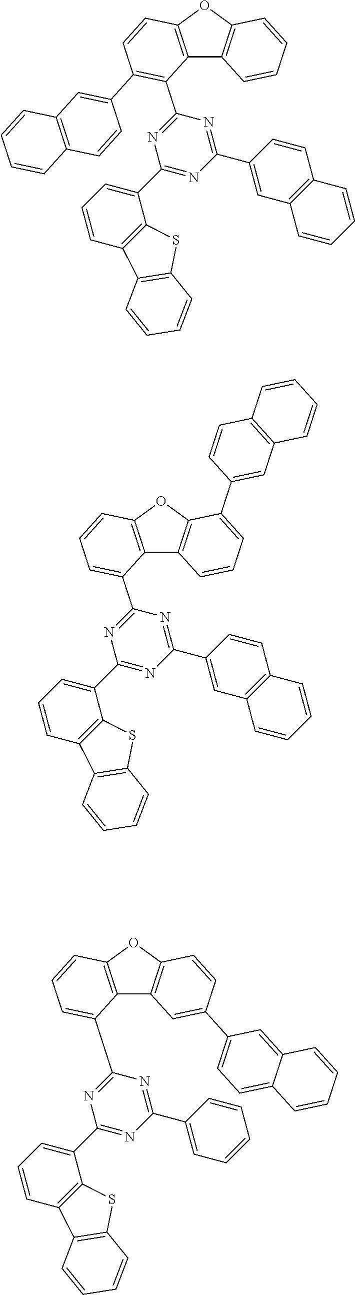 [SMILES: c1ccc2cc(-c3nc(-c4cccc5c4sc4ccccc45)nc(-c4c(-c5ccc6ccccc6c5)ccc5oc6ccccc6c45)n3)ccc2c1 ; c1ccc2cc(-c3nc(-c4cccc5c4sc4ccccc45)nc(-c4cccc5oc6c(-c7ccc8ccccc8c7)cccc6c45)n3)ccc2c1 ; c1ccc(-c2nc(-c3cccc4c3sc3ccccc34)nc(-c3cccc4oc5ccc(-c6ccc7ccccc7c6)cc5c34)n2)cc1]

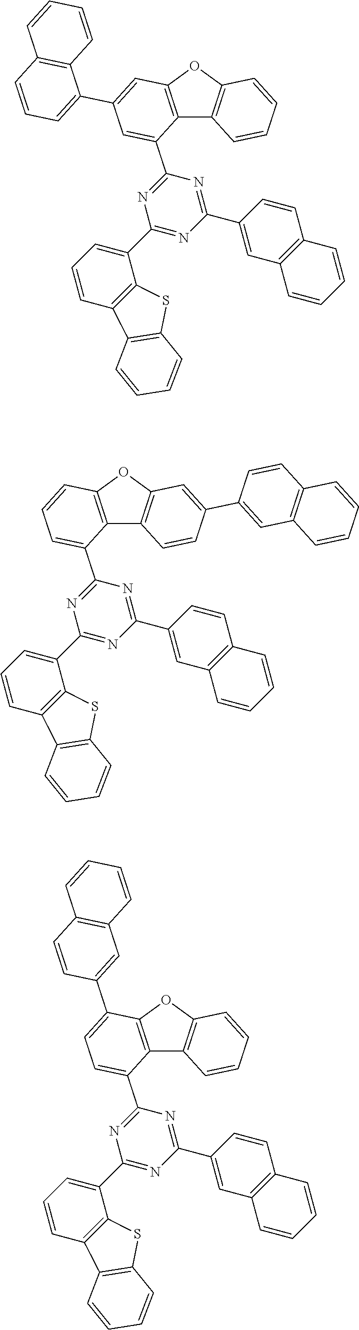 [SMILES: c1ccc2cc(-c3nc(-c4cccc5c4sc4ccccc45)nc(-c4cc(-c5cccc6ccccc56)cc5oc6ccccc6c45)n3)ccc2c1 ; c1ccc2cc(-c3ccc4c(c3)oc3cccc(-c5nc(-c6ccc7ccccc7c6)nc(-c6cccc7c6sc6ccccc67)n5)c34)ccc2c1 ; c1ccc2cc(-c3nc(-c4cccc5c4sc4ccccc45)nc(-c4ccc(-c5ccc6ccccc6c5)c5oc6ccccc6c45)n3)ccc2c1]

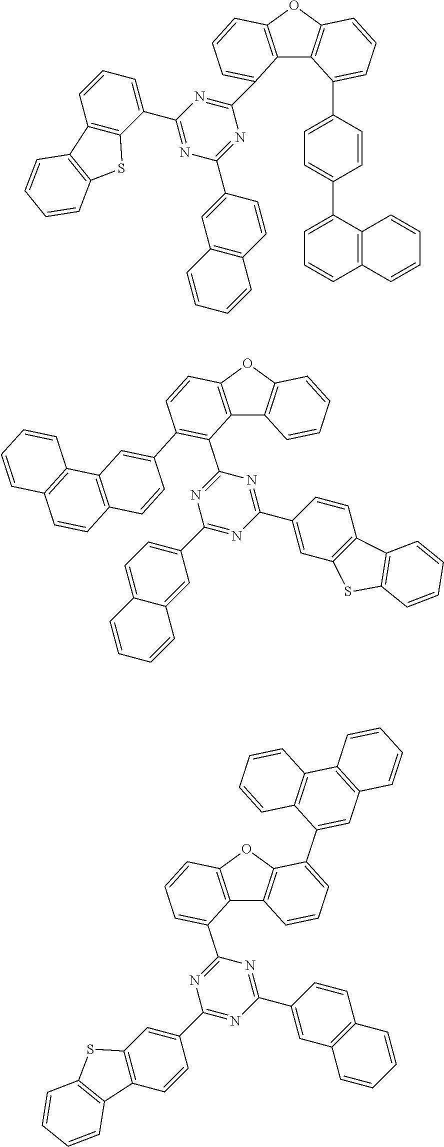 [SMILES: c1ccc2cc(-c3nc(-c4cccc5c4sc4ccccc45)nc(-c4cccc5oc6cccc(-c7ccc(-c8cccc9ccccc89)cc7)c6c45)n3)ccc2c1 ; c1ccc2cc(-c3nc(-c4ccc5c(c4)sc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)sc4ccccc45)nc(-c4cccc5oc6c(-c7cc8ccccc8c8ccccc78)cccc6c45)n3)ccc2c1]

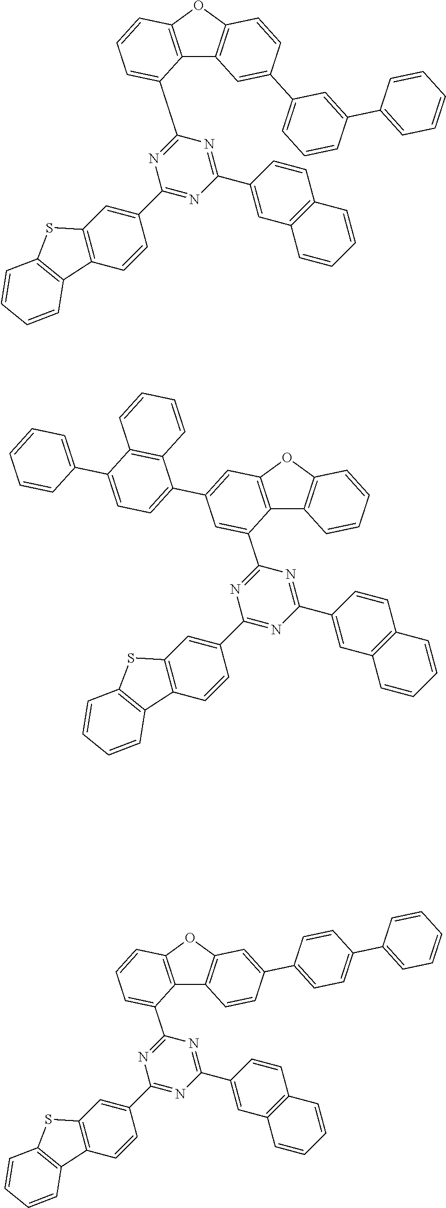 [SMILES: c1ccc(-c2cccc(-c3ccc4oc5cccc(-c6nc(-c7ccc8ccccc8c7)nc(-c7ccc8c(c7)sc7ccccc78)n6)c5c4c3)c2)cc1 ; c1ccc(-c2ccc(-c3cc(-c4nc(-c5ccc6ccccc6c5)nc(-c5ccc6c(c5)sc5ccccc56)n4)c4c(c3)oc3ccccc34)c3ccccc23)cc1 ; c1ccc(-c2ccc(-c3ccc4c(c3)oc3cccc(-c5nc(-c6ccc7ccccc7c6)nc(-c6ccc7c(c6)sc6ccccc67)n5)c34)cc2)cc1]

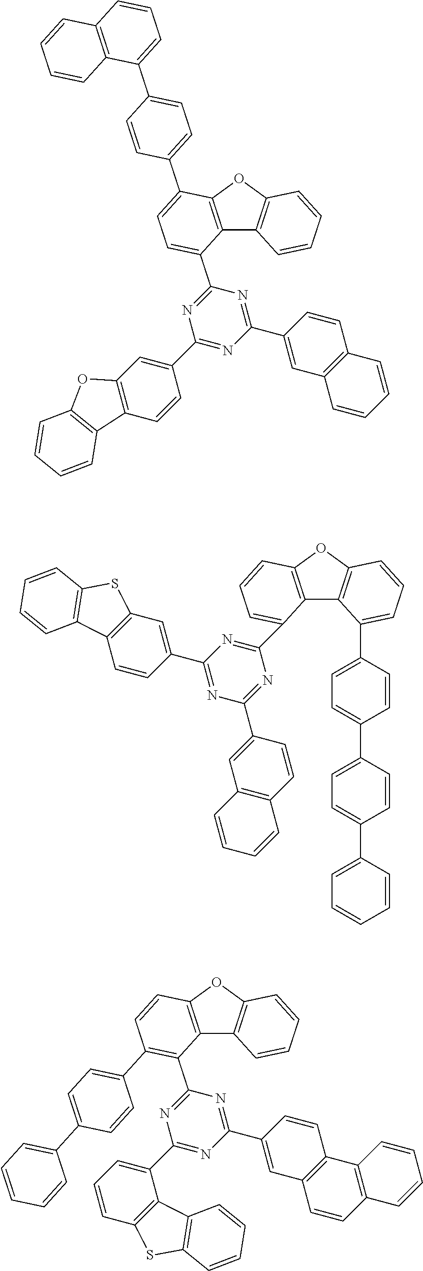 [SMILES: c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1 ; c1ccc(-c2ccc(-c3ccc(-c4cccc5oc6cccc(-c7nc(-c8ccc9ccccc9c8)nc(-c8ccc9c(c8)sc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5sc6ccccc6c45)n3)cc2)cc1]

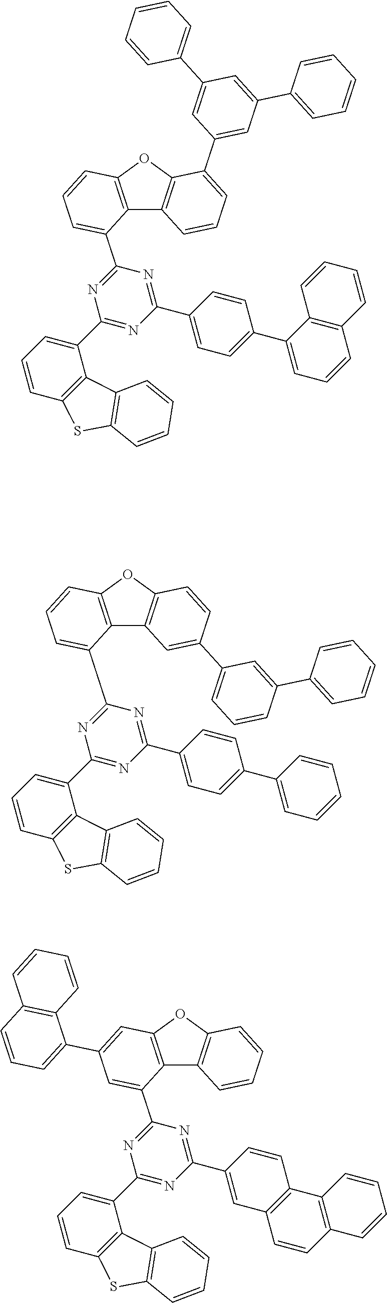 [SMILES: c1ccc(-c2cc(-c3ccccc3)cc(-c3cccc4c3oc3cccc(-c5nc(-c6ccc(-c7cccc8ccccc78)cc6)nc(-c6cccc7sc8ccccc8c67)n5)c34)c2)cc1 ; c1ccc(-c2ccc(-c3nc(-c4cccc5oc6ccc(-c7cccc(-c8ccccc8)c7)cc6c45)nc(-c4cccc5sc6ccccc6c45)n3)cc2)cc1 ; c1ccc2c(-c3cc(-c4nc(-c5ccc6c(ccc7ccccc76)c5)nc(-c5cccc6sc7ccccc7c56)n4)c4c(c3)oc3ccccc34)cccc2c1]

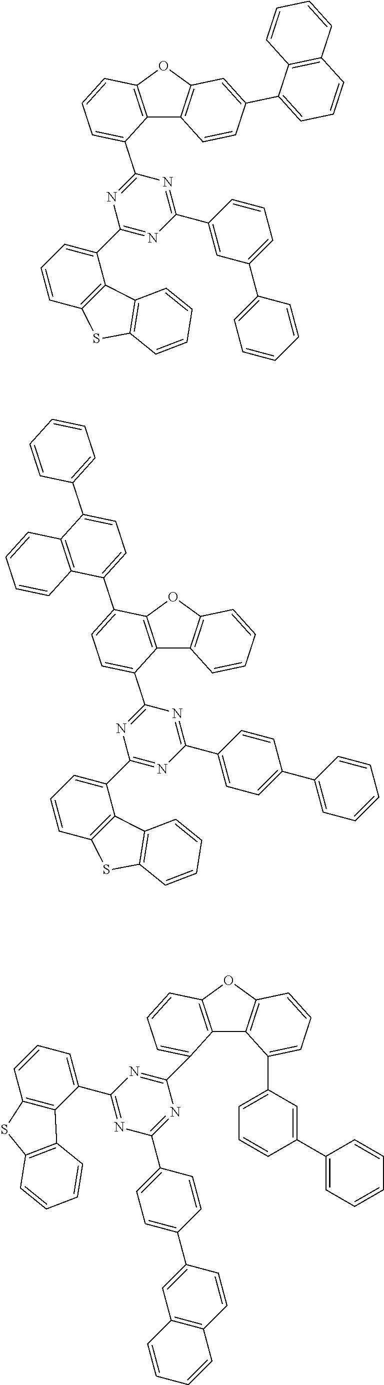 [SMILES: c1ccc(-c2cccc(-c3nc(-c4cccc5oc6cc(-c7cccc8ccccc78)ccc6c45)nc(-c4cccc5sc6ccccc6c45)n3)c2)cc1 ; c1ccc(-c2ccc(-c3nc(-c4cccc5sc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8sc9ccccc9c78)n6)c5c34)c2)cc1]

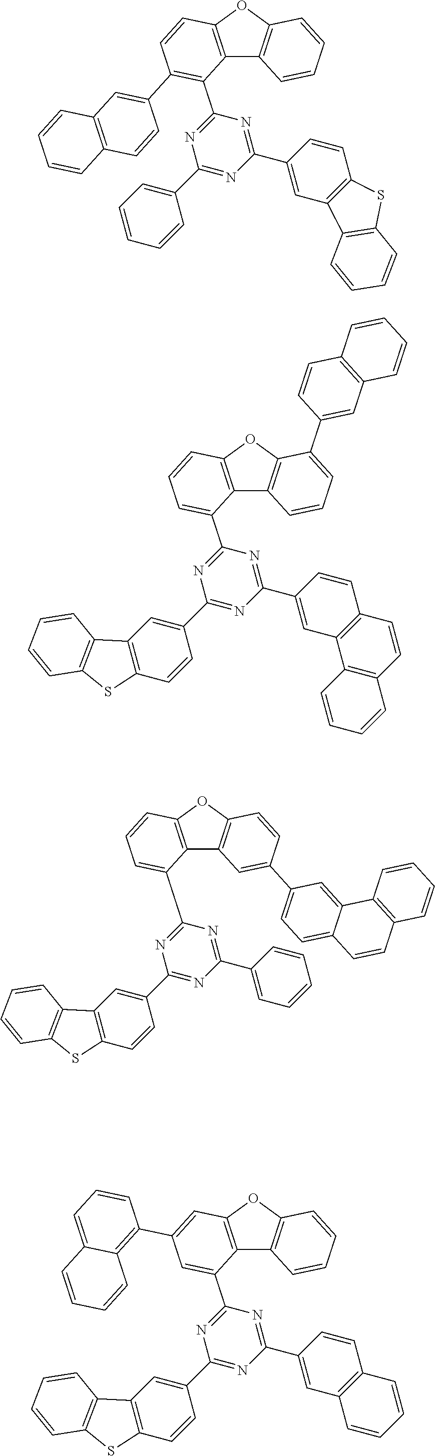

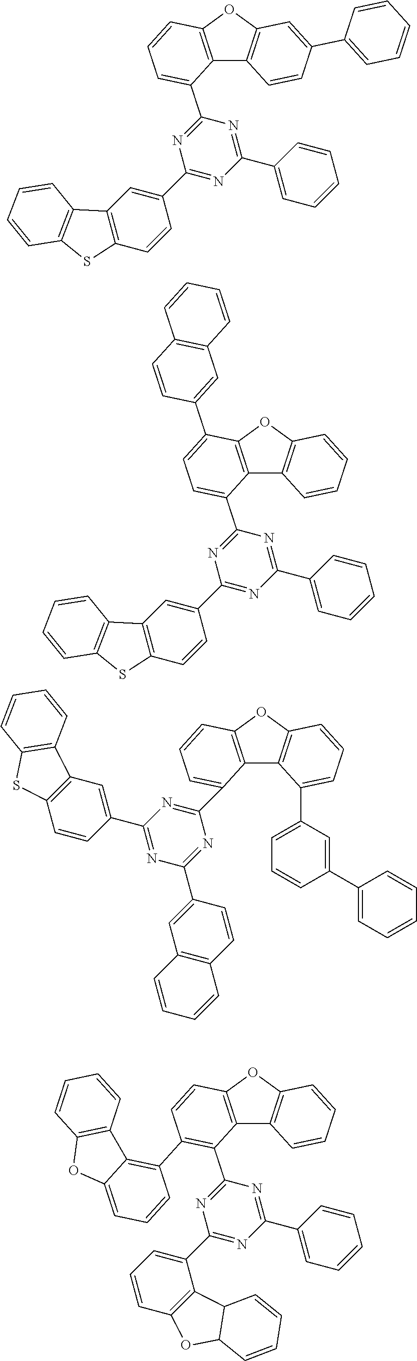

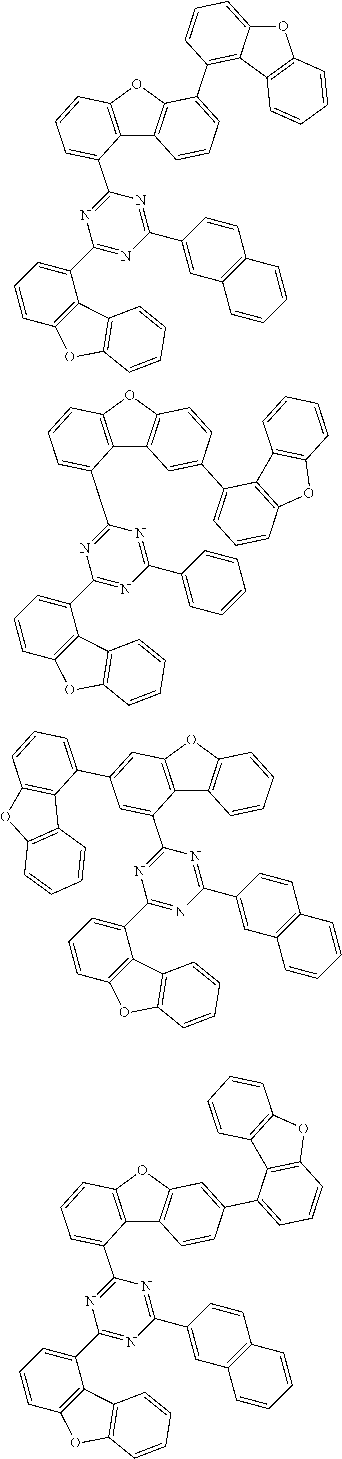

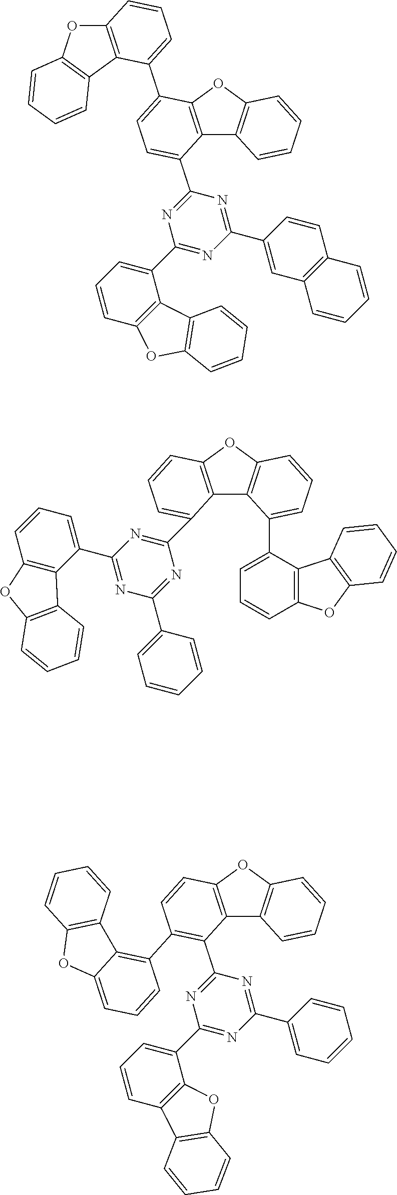 [SMILES: c1ccc2cc(-c3nc(-c4cccc5oc6ccccc6c45)nc(-c4ccc(-c5cccc6oc7ccccc7c56)c5oc6ccccc6c45)n3)ccc2c1 ; c1ccc(-c2nc(-c3cccc4oc5ccccc5c34)nc(-c3cccc4oc5cccc(-c6cccc7oc8ccccc8c67)c5c34)n2)cc1 ; c1ccc(-c2nc(-c3cccc4c3oc3ccccc34)nc(-c3c(-c4cccc5oc6ccccc6c45)ccc4oc5ccccc5c34)n2)cc1]

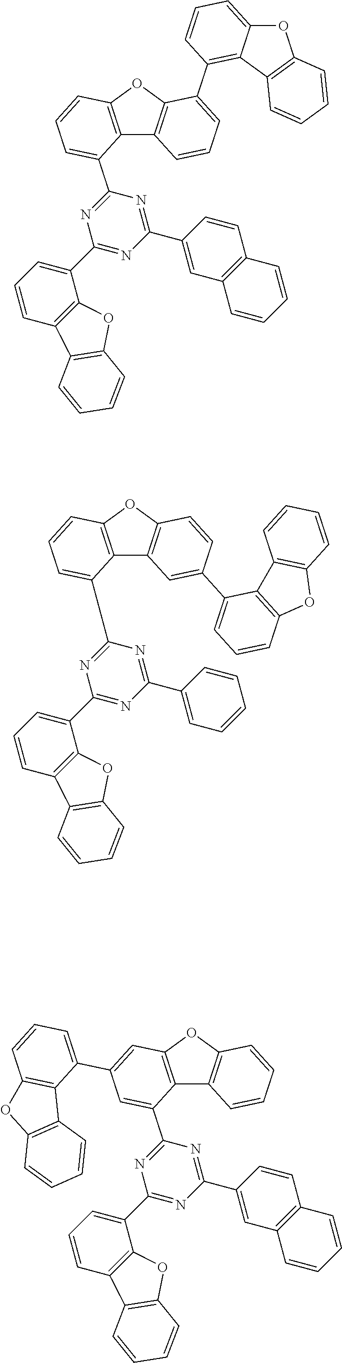 [SMILES: c1ccc2cc(-c3nc(-c4cccc5c4oc4ccccc45)nc(-c4cccc5oc6c(-c7cccc8oc9ccccc9c78)cccc6c45)n3)ccc2c1 ; c1ccc(-c2nc(-c3cccc4c3oc3ccccc34)nc(-c3cccc4oc5ccc(-c6cccc7oc8ccccc8c67)cc5c34)n2)cc1 ; c1ccc2cc(-c3nc(-c4cccc5c4oc4ccccc45)nc(-c4cc(-c5cccc6oc7ccccc7c56)cc5oc6ccccc6c45)n3)ccc2c1]

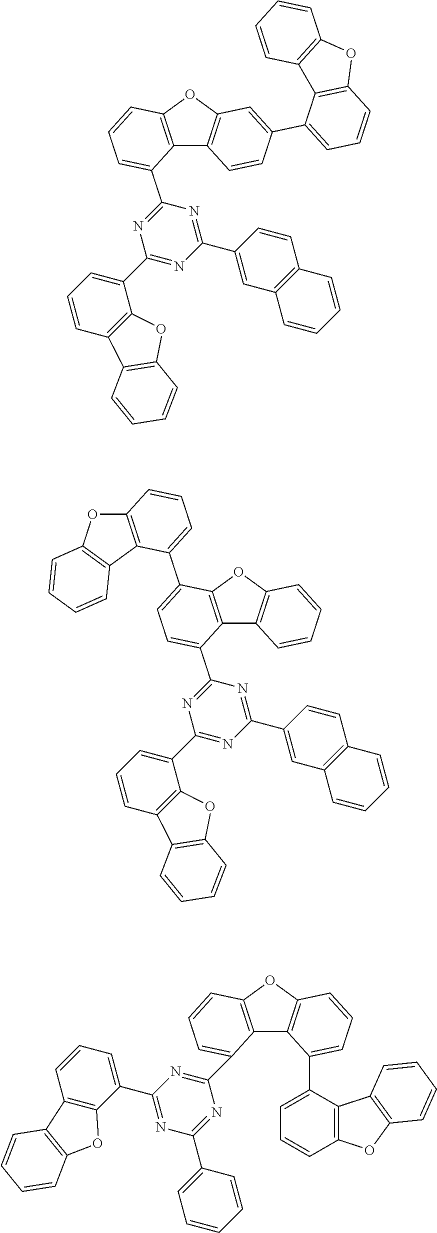 [SMILES: c1ccc2cc(-c3nc(-c4cccc5c4oc4ccccc45)nc(-c4cccc5oc6cc(-c7cccc8oc9ccccc9c78)ccc6c45)n3)ccc2c1 ; c1ccc2cc(-c3nc(-c4cccc5c4oc4ccccc45)nc(-c4ccc(-c5cccc6oc7ccccc7c56)c5oc6ccccc6c45)n3)ccc2c1 ; c1ccc(-c2nc(-c3cccc4c3oc3ccccc34)nc(-c3cccc4oc5cccc(-c6cccc7oc8ccccc8c67)c5c34)n2)cc1]

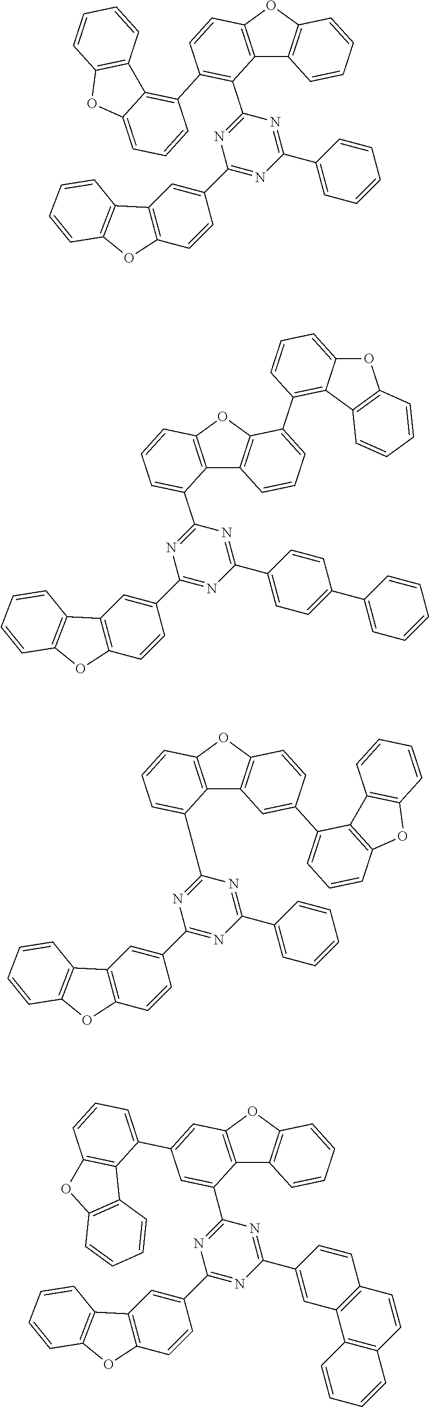 [SMILES: c1ccc(-c2nc(-c3ccc4oc5ccccc5c4c3)nc(-c3c(-c4cccc5oc6ccccc6c45)ccc4oc5ccccc5c34)n2)cc1 ; c1ccc(-c2ccc(-c3nc(-c4ccc5oc6ccccc6c5c4)nc(-c4cccc5oc6c(-c7cccc8oc9ccccc9c78)cccc6c45)n3)cc2)cc1 ; c1ccc(-c2nc(-c3ccc4oc5ccccc5c4c3)nc(-c3cccc4oc5ccc(-c6cccc7oc8ccccc8c67)cc5c34)n2)cc1 ; c1ccc2c(c1)ccc1ccc(-c3nc(-c4ccc5oc6ccccc6c5c4)nc(-c4cc(-c5cccc6oc7ccccc7c56)cc5oc6ccccc6c45)n3)cc12]

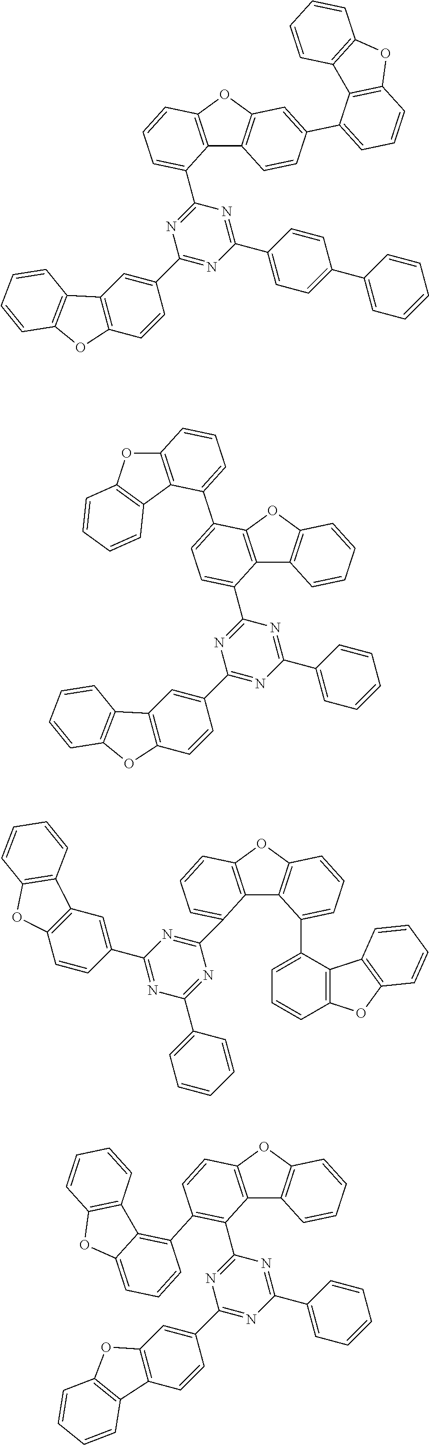 [SMILES: c1ccc(-c2ccc(-c3nc(-c4ccc5oc6ccccc6c5c4)nc(-c4cccc5oc6cc(-c7cccc8oc9ccccc9c78)ccc6c45)n3)cc2)cc1 ; c1ccc(-c2nc(-c3ccc4oc5ccccc5c4c3)nc(-c3ccc(-c4cccc5oc6ccccc6c45)c4oc5ccccc5c34)n2)cc1 ; c1ccc(-c2nc(-c3ccc4oc5ccccc5c4c3)nc(-c3cccc4oc5cccc(-c6cccc7oc8ccccc8c67)c5c34)n2)cc1 ; c1ccc(-c2nc(-c3ccc4c(c3)oc3ccccc34)nc(-c3c(-c4cccc5oc6ccccc6c45)ccc4oc5ccccc5c34)n2)cc1]

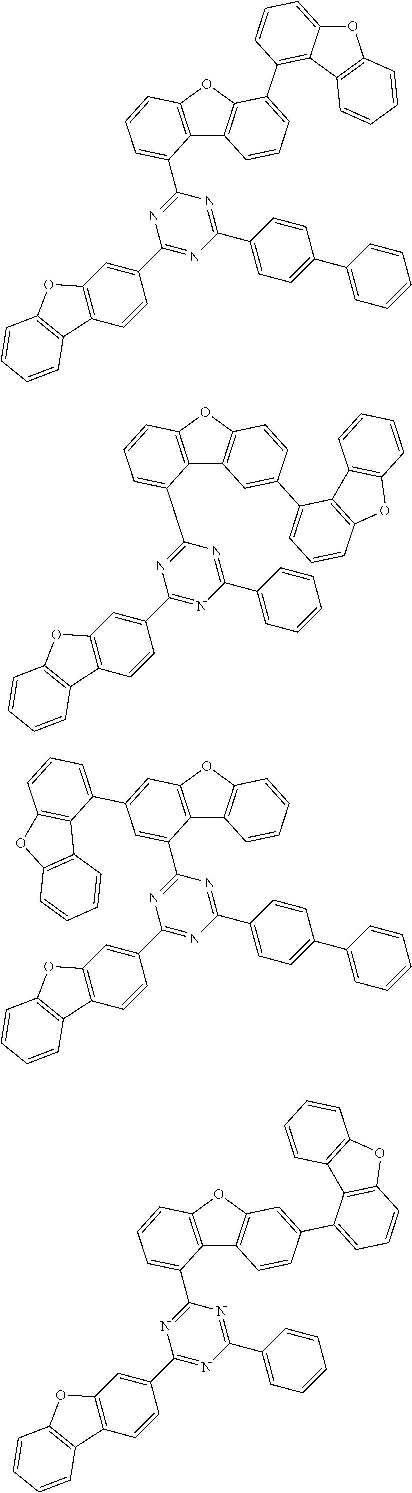

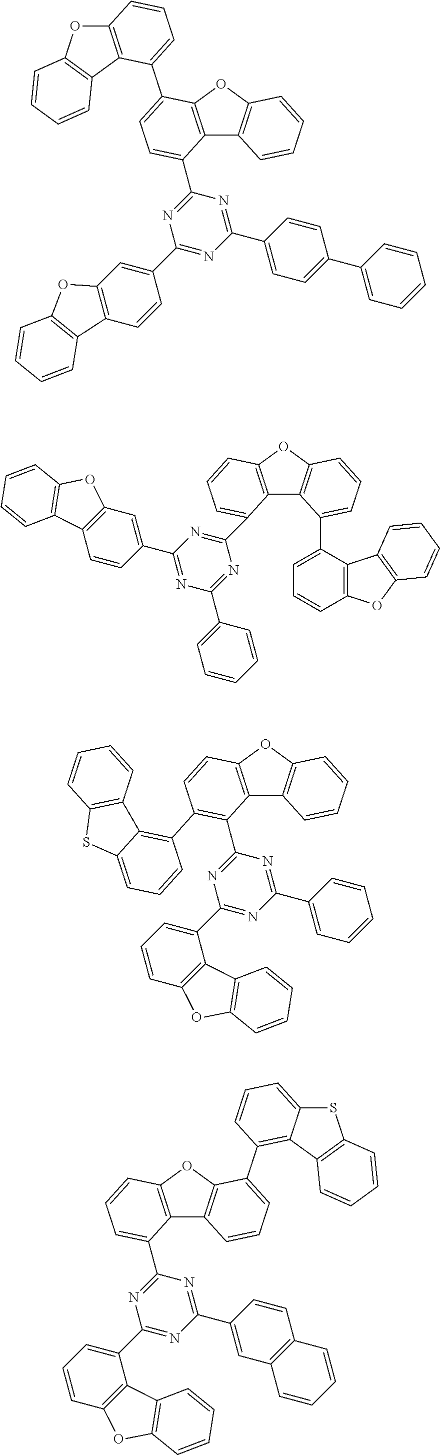 [SMILES: c1ccc(-c2ccc(-c3nc(-c4ccc5c(c4)oc4ccccc45)nc(-c4ccc(-c5cccc6oc7ccccc7c56)c5oc6ccccc6c45)n3)cc2)cc1 ; c1ccc(-c2nc(-c3ccc4c(c3)oc3ccccc34)nc(-c3cccc4oc5cccc(-c6cccc7oc8ccccc8c67)c5c34)n2)cc1 ; c1ccc(-c2nc(-c3cccc4oc5ccccc5c34)nc(-c3c(-c4cccc5sc6ccccc6c45)ccc4oc5ccccc5c34)n2)cc1 ; c1ccc2cc(-c3nc(-c4cccc5oc6ccccc6c45)nc(-c4cccc5oc6c(-c7cccc8sc9ccccc9c78)cccc6c45)n3)ccc2c1]

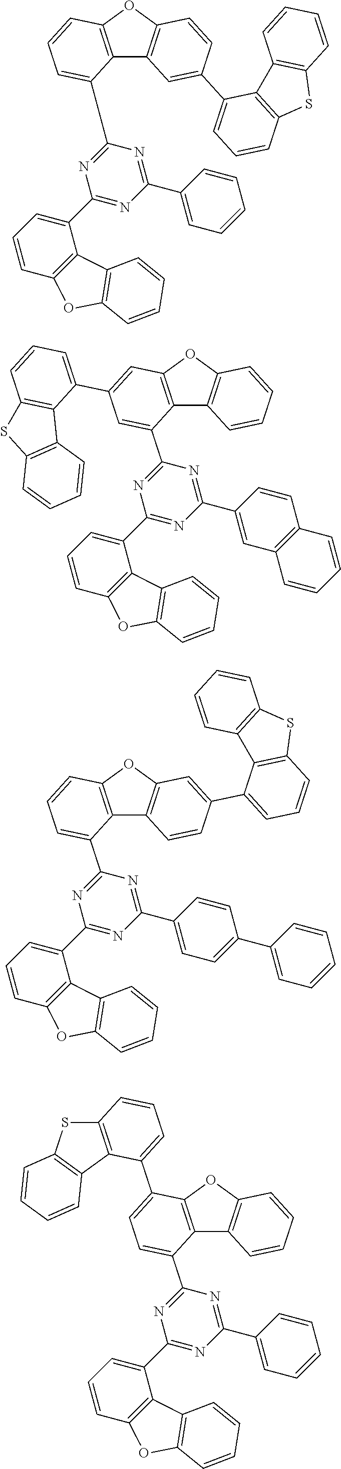

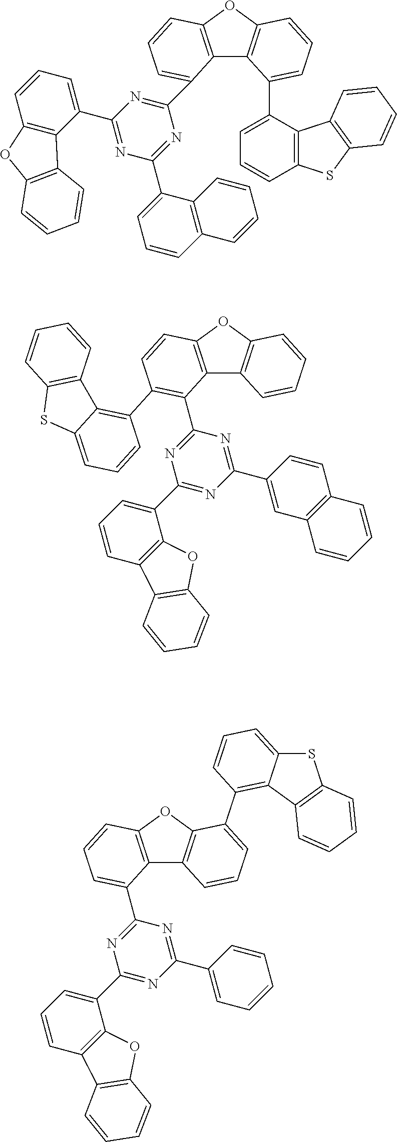 [SMILES: c1ccc2c(-c3nc(-c4cccc5oc6ccccc6c45)nc(-c4cccc5oc6cccc(-c7cccc8sc9ccccc9c78)c6c45)n3)cccc2c1 ; c1ccc2cc(-c3nc(-c4cccc5c4oc4ccccc45)nc(-c4c(-c5cccc6sc7ccccc7c56)ccc5oc6ccccc6c45)n3)ccc2c1 ; c1ccc(-c2nc(-c3cccc4c3oc3ccccc34)nc(-c3cccc4oc5c(-c6cccc7sc8ccccc8c67)cccc5c34)n2)cc1]

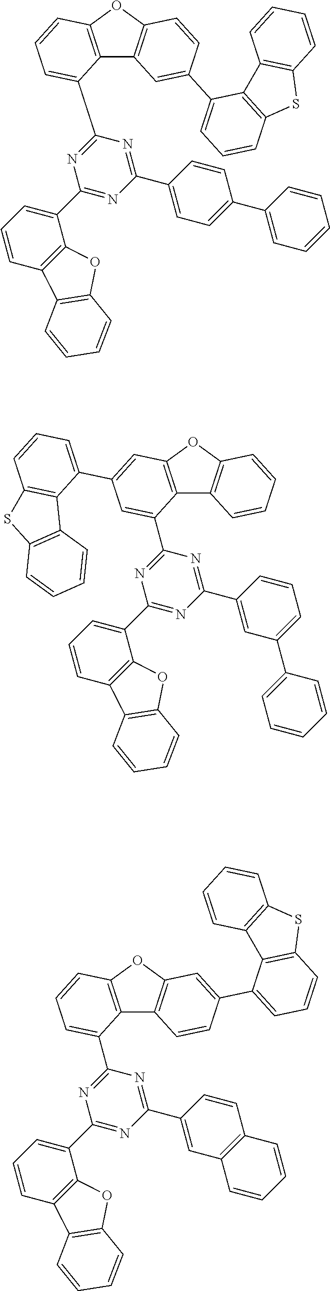 [SMILES: c1ccc(-c2ccc(-c3nc(-c4cccc5c4oc4ccccc45)nc(-c4cccc5oc6ccc(-c7cccc8sc9ccccc9c78)cc6c45)n3)cc2)cc1 ; c1ccc(-c2cccc(-c3nc(-c4cccc5c4oc4ccccc45)nc(-c4cc(-c5cccc6sc7ccccc7c56)cc5oc6ccccc6c45)n3)c2)cc1 ; c1ccc2cc(-c3nc(-c4cccc5c4oc4ccccc45)nc(-c4cccc5oc6cc(-c7cccc8sc9ccccc9c78)ccc6c45)n3)ccc2c1]

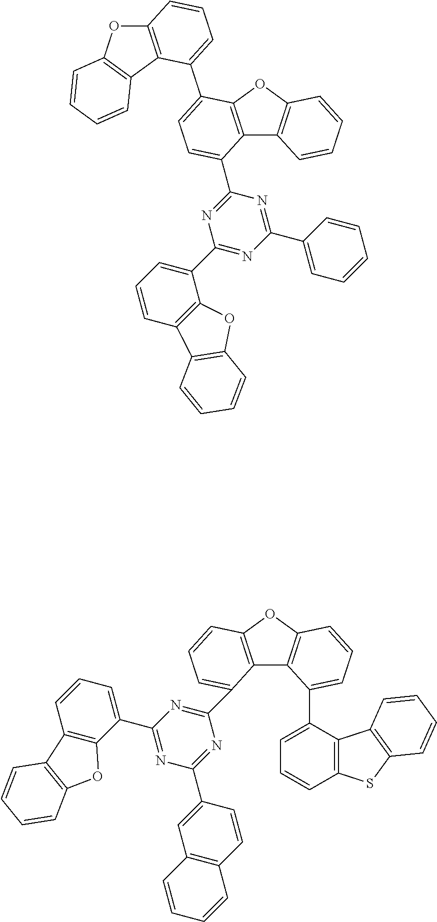 [SMILES: c1ccc(-c2nc(-c3cccc4c3oc3ccccc34)nc(-c3ccc(-c4cccc5oc6ccccc6c45)c4oc5ccccc5c34)n2)cc1 ; c1ccc2cc(-c3nc(-c4cccc5c4oc4ccccc45)nc(-c4cccc5oc6cccc(-c7cccc8sc9ccccc9c78)c6c45)n3)ccc2c1]

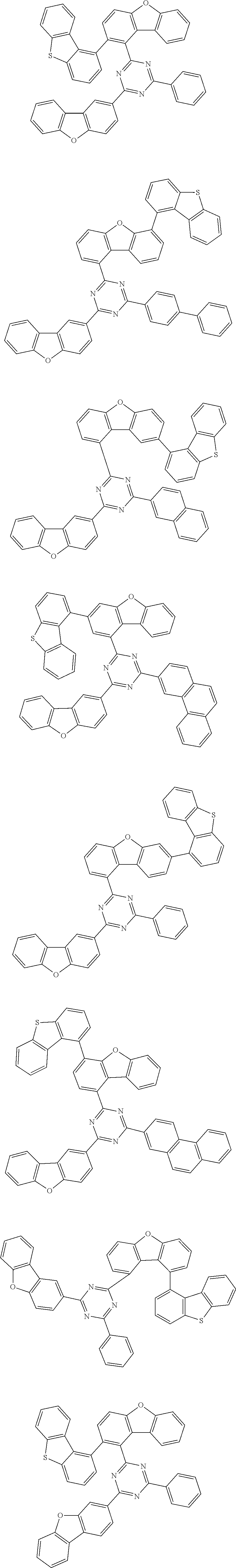

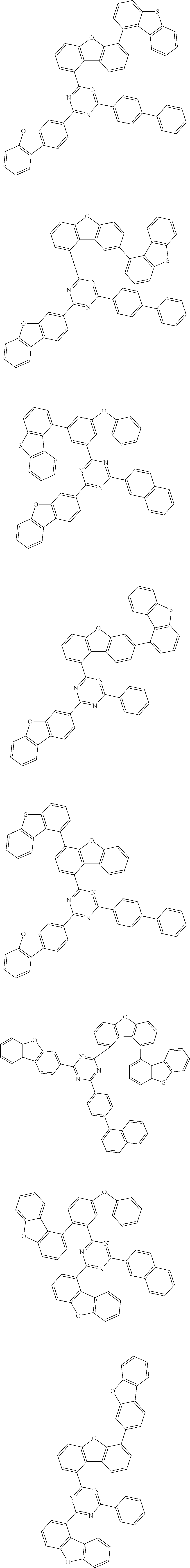

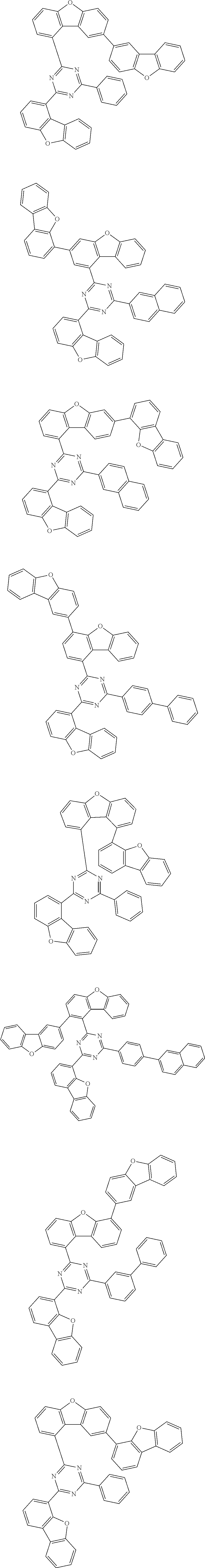

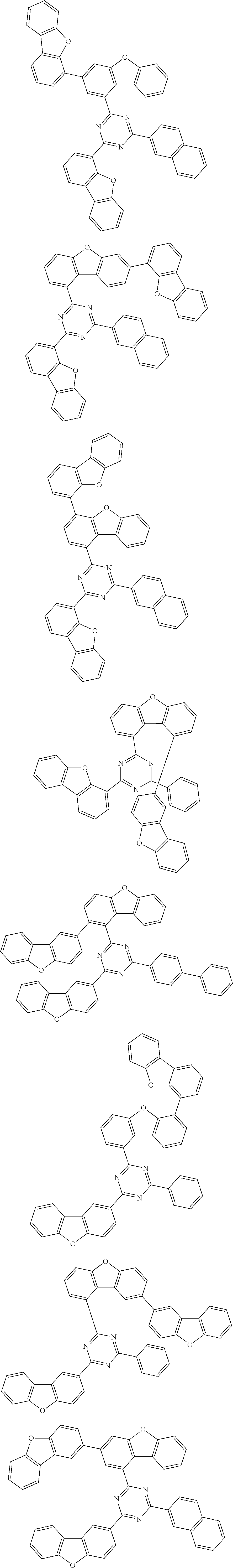

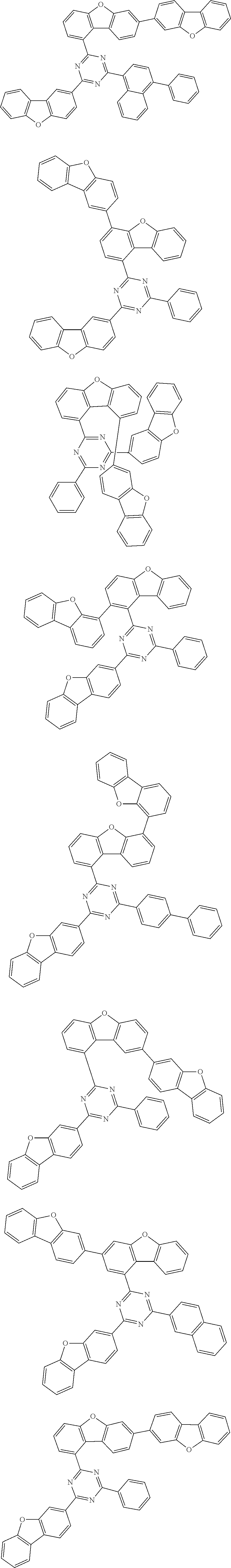

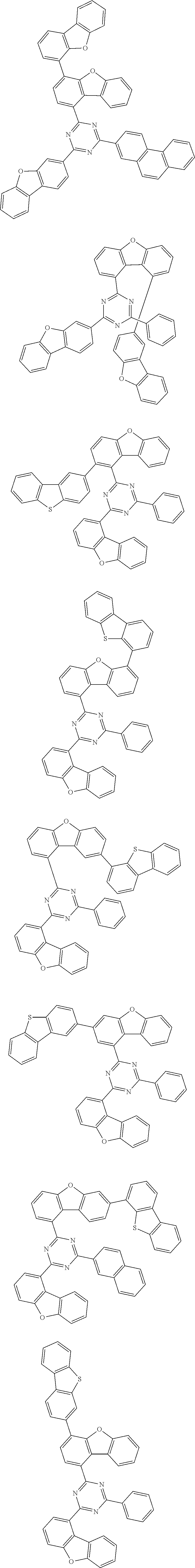

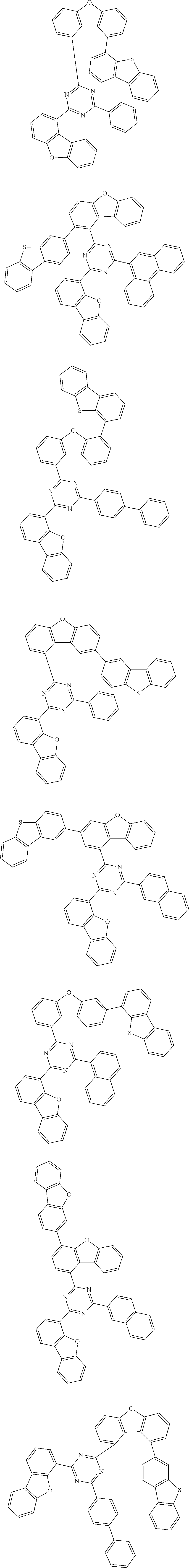

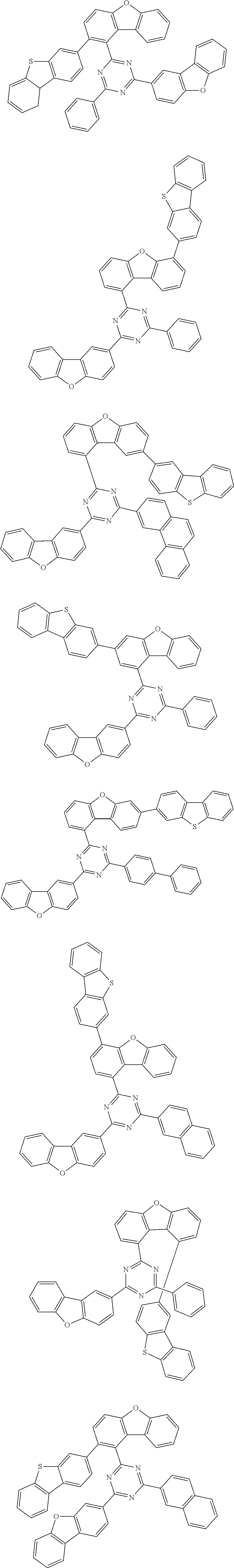

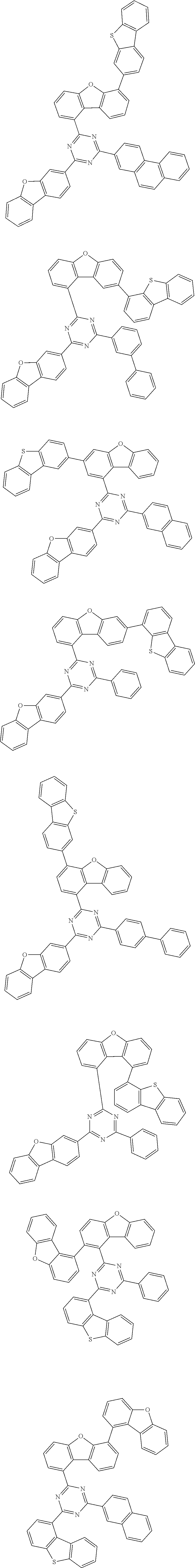

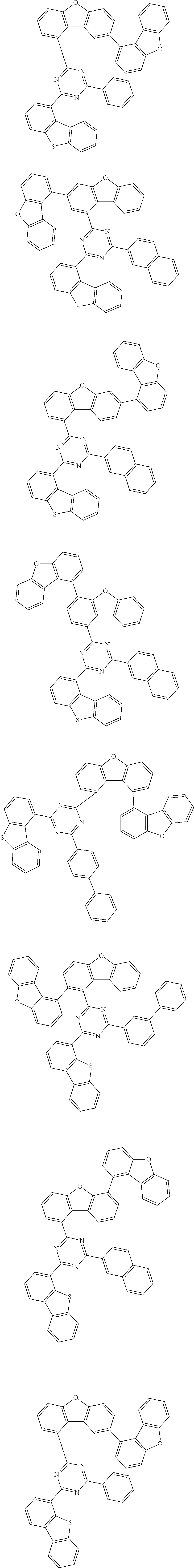

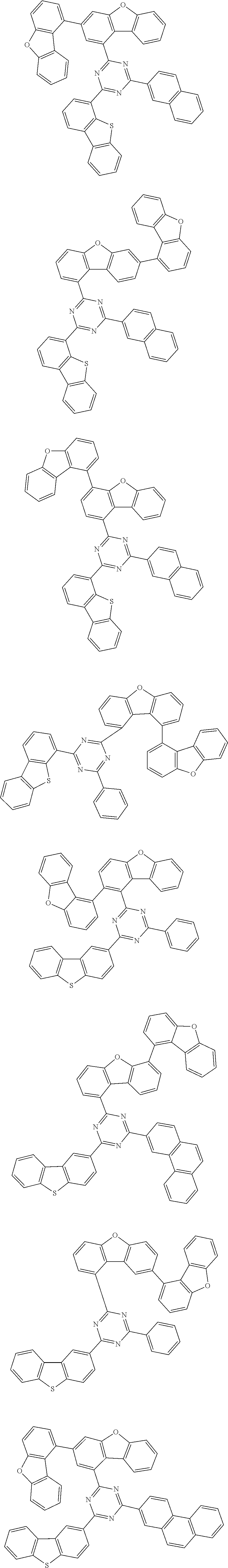

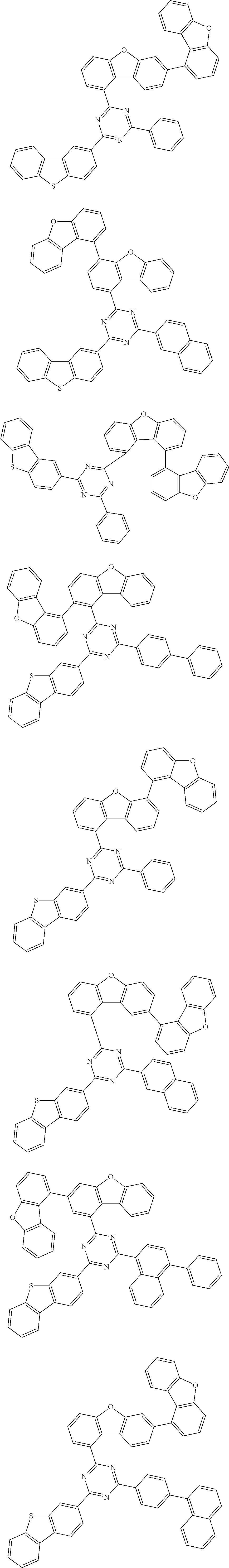

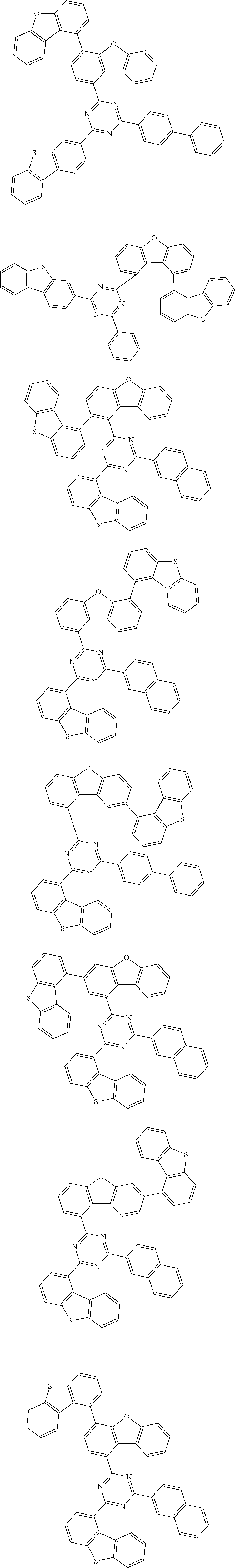

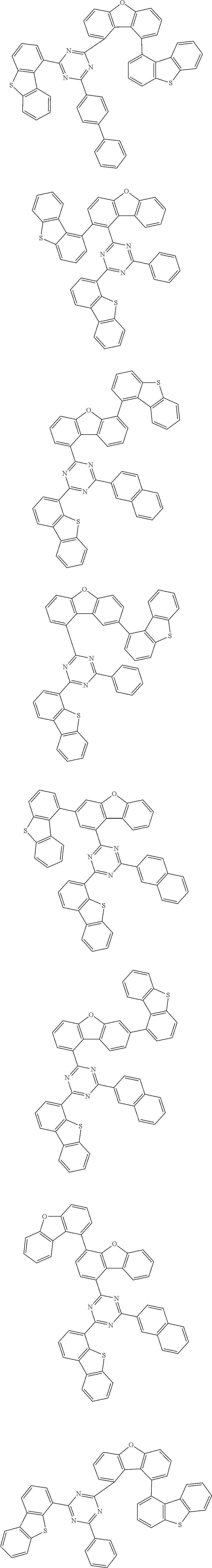

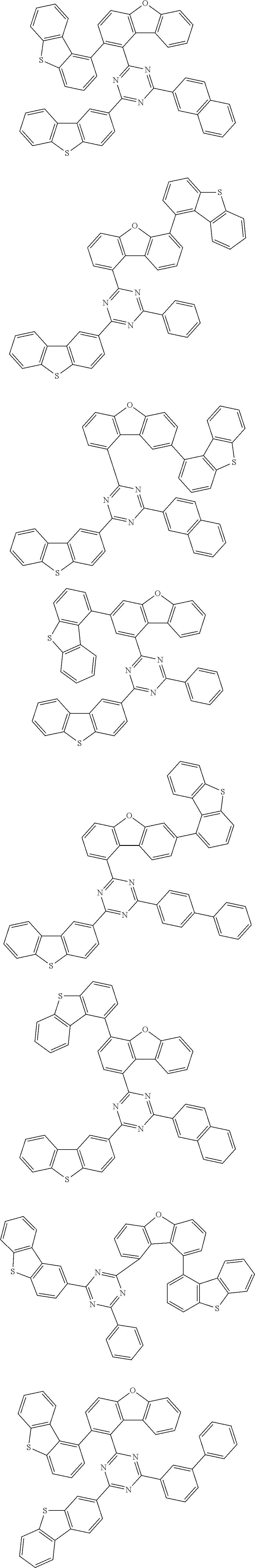

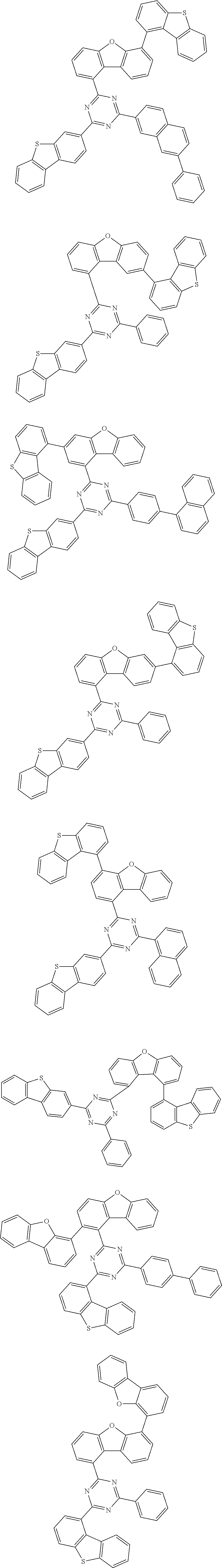

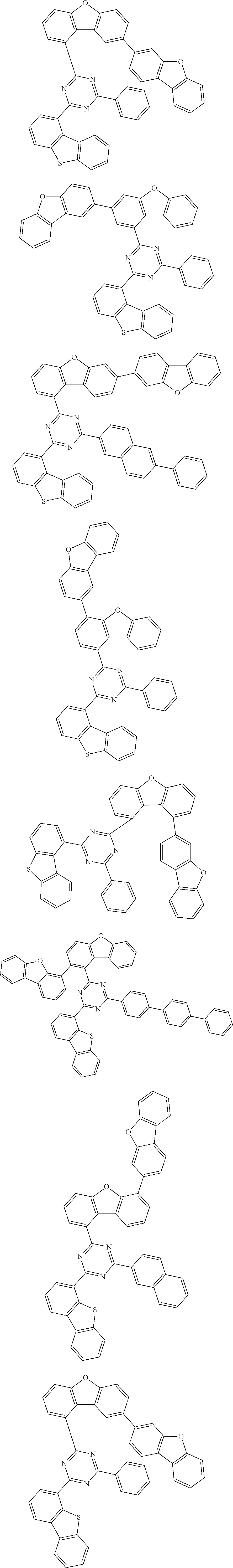

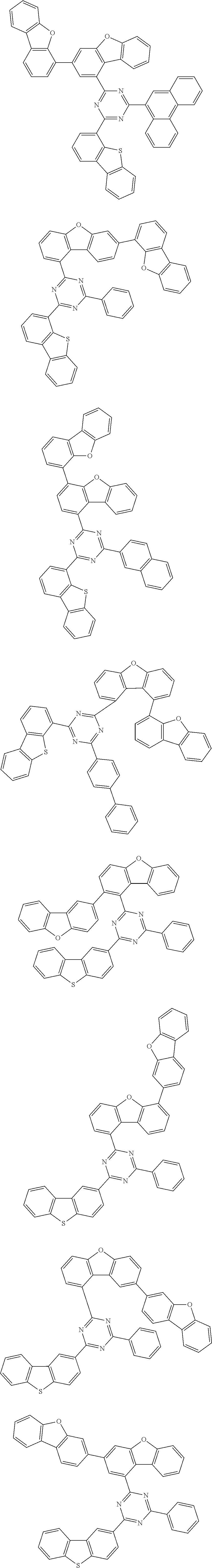

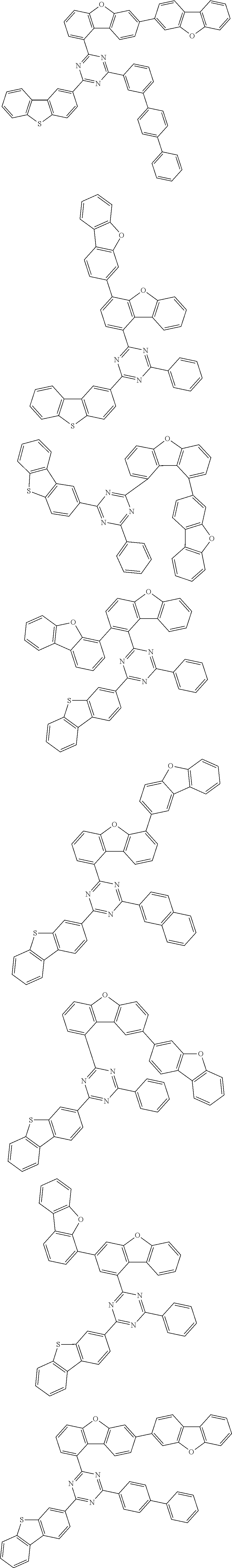

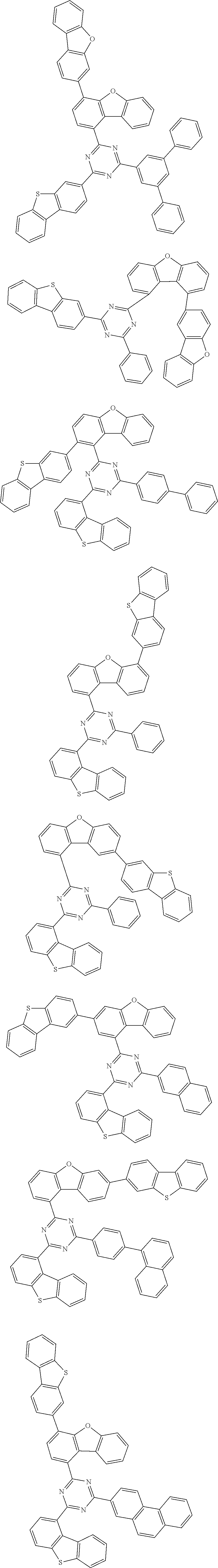

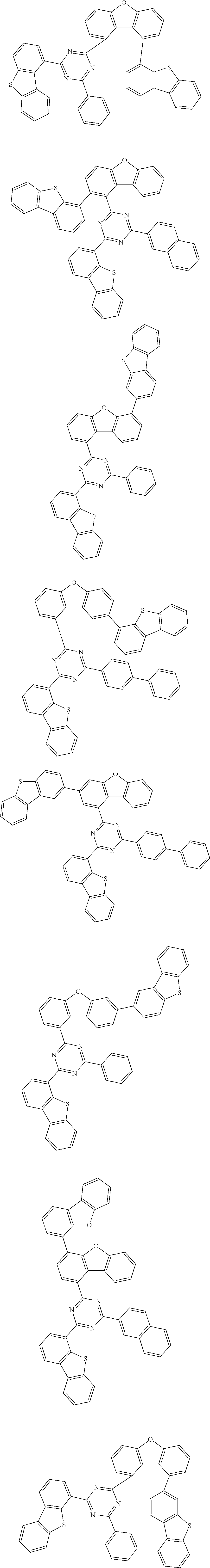

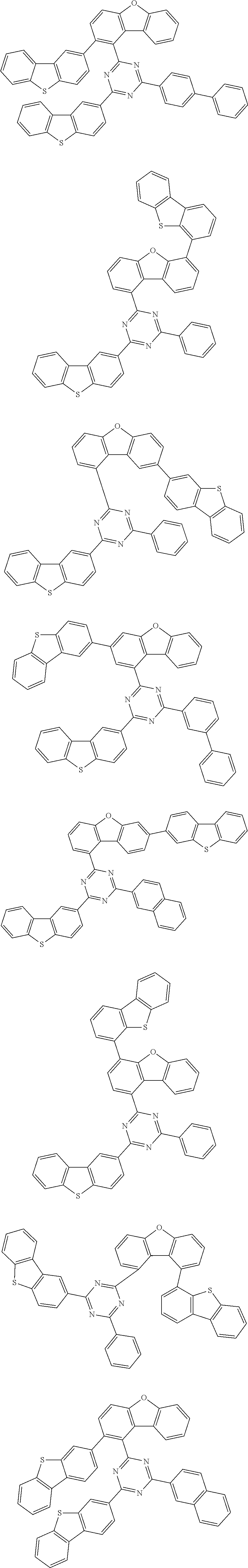

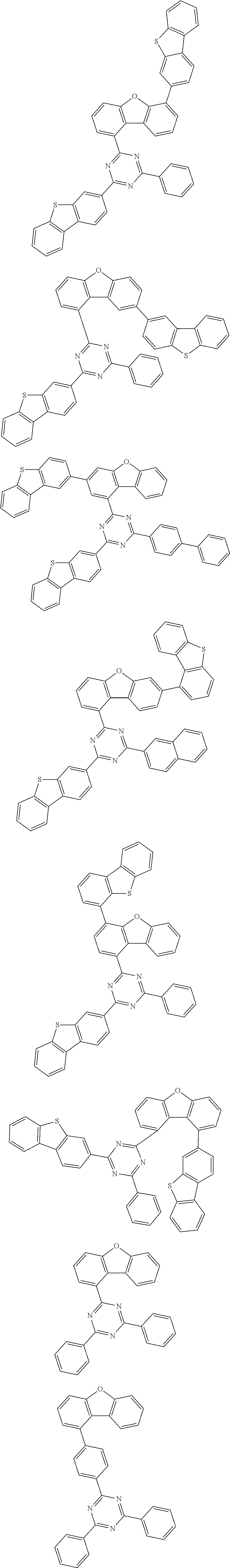

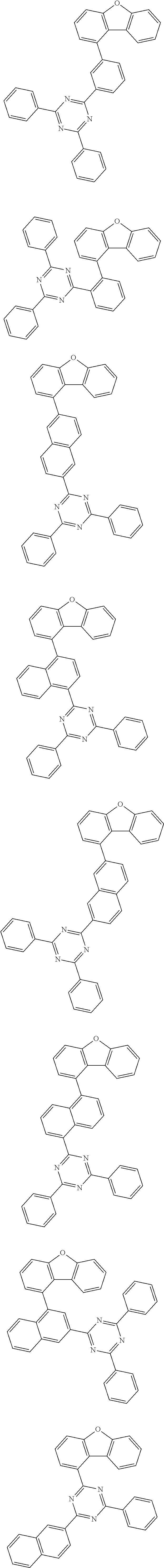

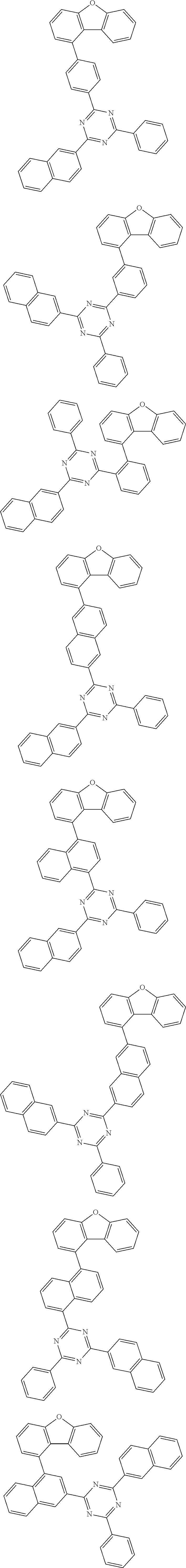

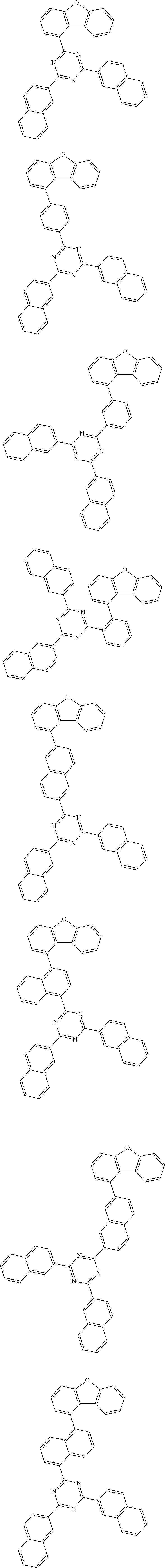

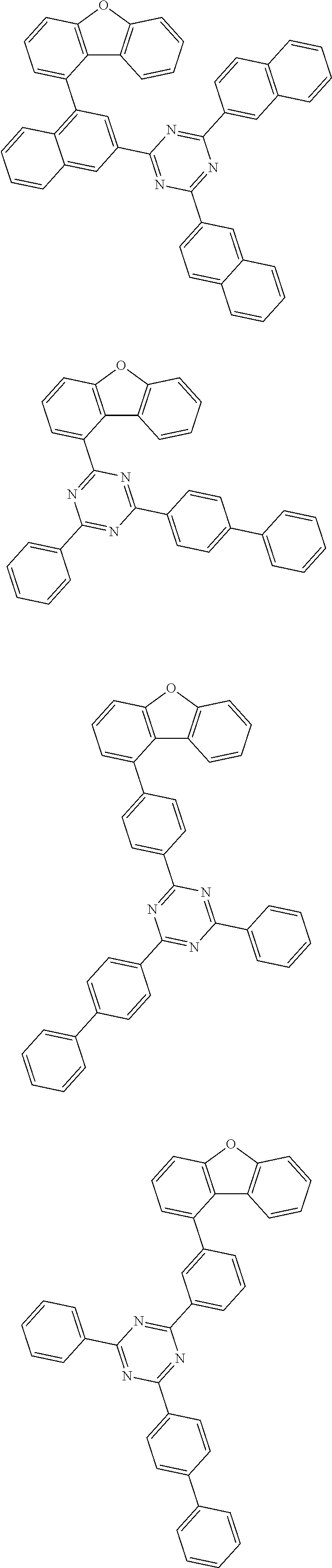 [SMILES: c1ccc2cc(-c3nc(-c4ccc5ccccc5c4)nc(-c4cc(-c5cccc6oc7ccccc7c56)c5ccccc5c4)n3)ccc2c1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1]

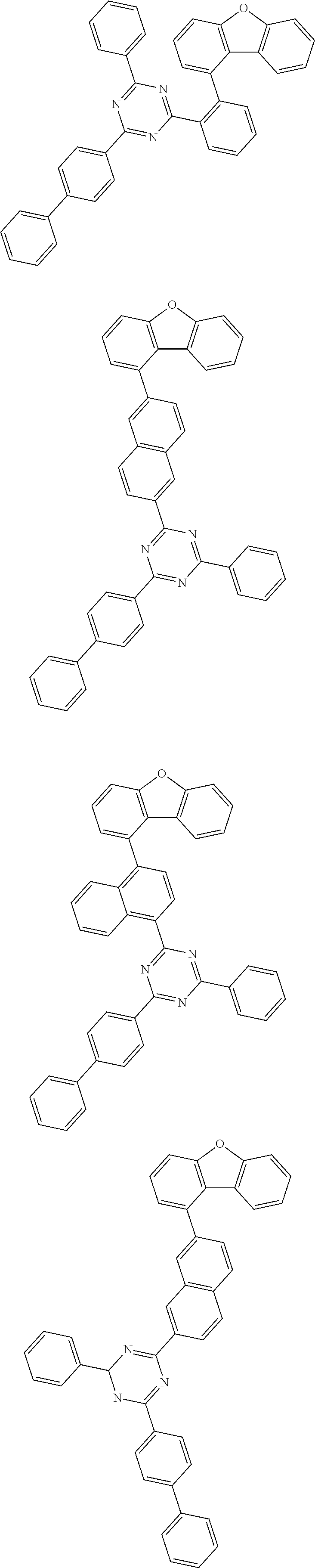

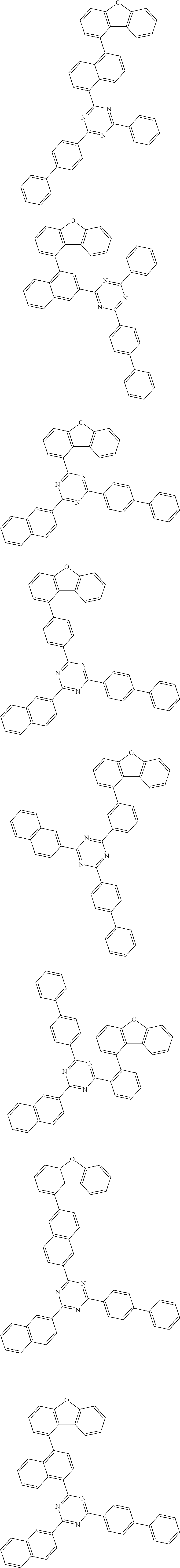

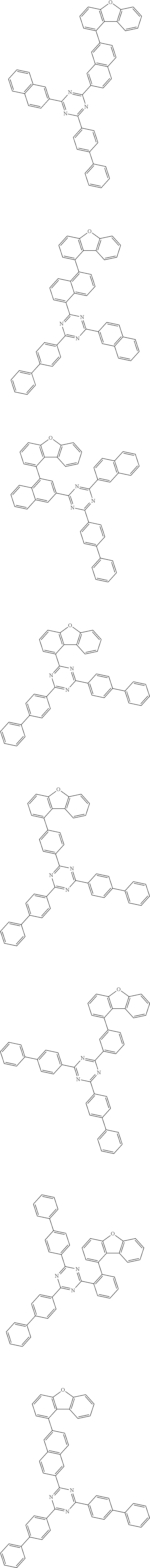

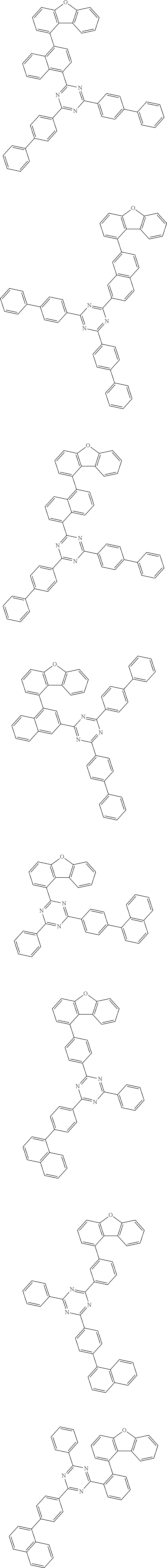

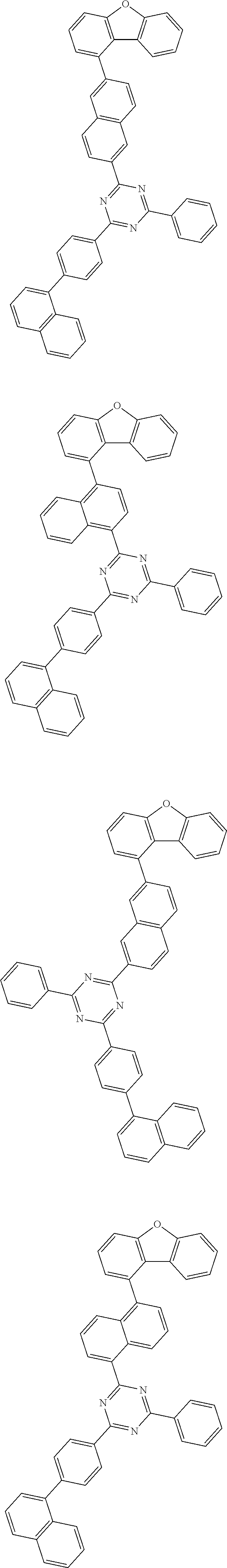 [SMILES: c1ccc(-c2nc(-c3ccc(-c4cccc5ccccc45)cc3)nc(-c3ccc4cc(-c5cccc6oc7ccccc7c56)ccc4c3)n2)cc1 ; c1ccc(-c2nc(-c3ccc(-c4cccc5ccccc45)cc3)nc(-c3ccc(-c4cccc5oc6ccccc6c45)c4ccccc34)n2)cc1 ; c1ccc(-c2nc(-c3ccc(-c4cccc5ccccc45)cc3)nc(-c3ccc4ccc(-c5cccc6oc7ccccc7c56)cc4c3)n2)cc1 ; c1ccc(-c2nc(-c3ccc(-c4cccc5ccccc45)cc3)nc(-c3cccc4c(-c5cccc6oc7ccccc7c56)cccc34)n2)cc1]

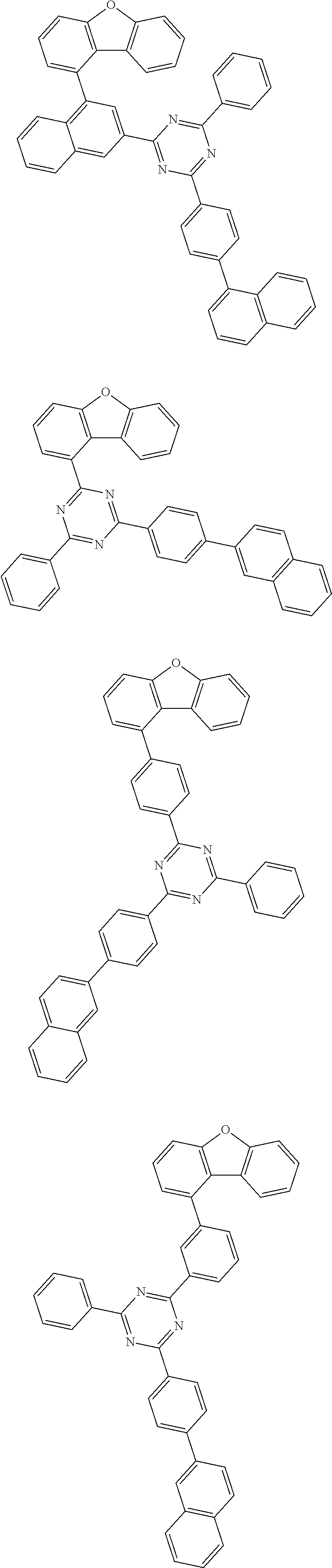 [SMILES: c1ccc(-c2nc(-c3ccc(-c4cccc5ccccc45)cc3)nc(-c3cc(-c4cccc5oc6ccccc6c45)c4ccccc4c3)n2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc(-c4cccc5oc6ccccc6c45)cc3)n2)cc1 ; c1ccc(-c2nc(-c3ccc(-c4ccc5ccccc5c4)cc3)nc(-c3cccc(-c4cccc5oc6ccccc6c45)c3)n2)cc1]

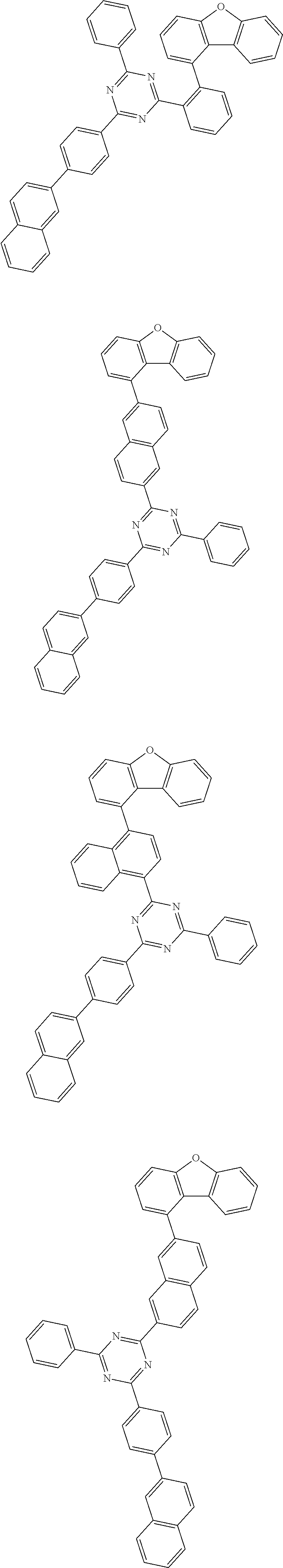 [SMILES: c1ccc(-c2nc(-c3ccc(-c4ccc5ccccc5c4)cc3)nc(-c3ccccc3-c3cccc4oc5ccccc5c34)n2)cc1 ; c1ccc(-c2nc(-c3ccc(-c4ccc5ccccc5c4)cc3)nc(-c3ccc4cc(-c5cccc6oc7ccccc7c56)ccc4c3)n2)cc1 ; c1ccc(-c2nc(-c3ccc(-c4ccc5ccccc5c4)cc3)nc(-c3ccc(-c4cccc5oc6ccccc6c45)c4ccccc34)n2)cc1 ; c1ccc(-c2nc(-c3ccc(-c4ccc5ccccc5c4)cc3)nc(-c3ccc4ccc(-c5cccc6oc7ccccc7c56)cc4c3)n2)cc1]

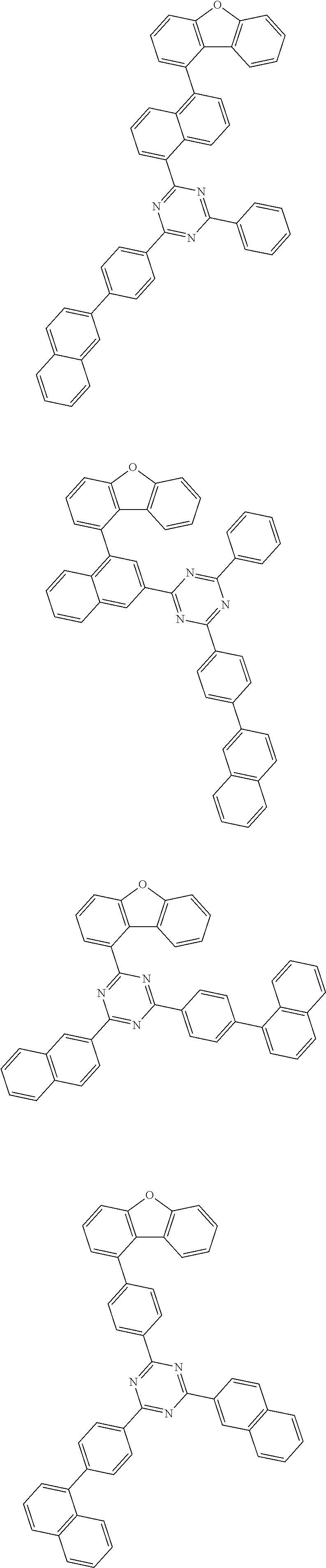 [SMILES: c1ccc(-c2nc(-c3ccc(-c4ccc5ccccc5c4)cc3)nc(-c3cccc4c(-c5cccc6oc7ccccc7c56)cccc34)n2)cc1 ; c1ccc(-c2nc(-c3ccc(-c4ccc5ccccc5c4)cc3)nc(-c3cc(-c4cccc5oc6ccccc6c45)c4ccccc4c3)n2)cc1 ; c1ccc2cc(-c3nc(-c4ccc(-c5cccc6ccccc56)cc4)nc(-c4cccc5oc6ccccc6c45)n3)ccc2c1 ; c1ccc2cc(-c3nc(-c4ccc(-c5cccc6ccccc56)cc4)nc(-c4ccc(-c5cccc6oc7ccccc7c56)cc4)n3)ccc2c1]

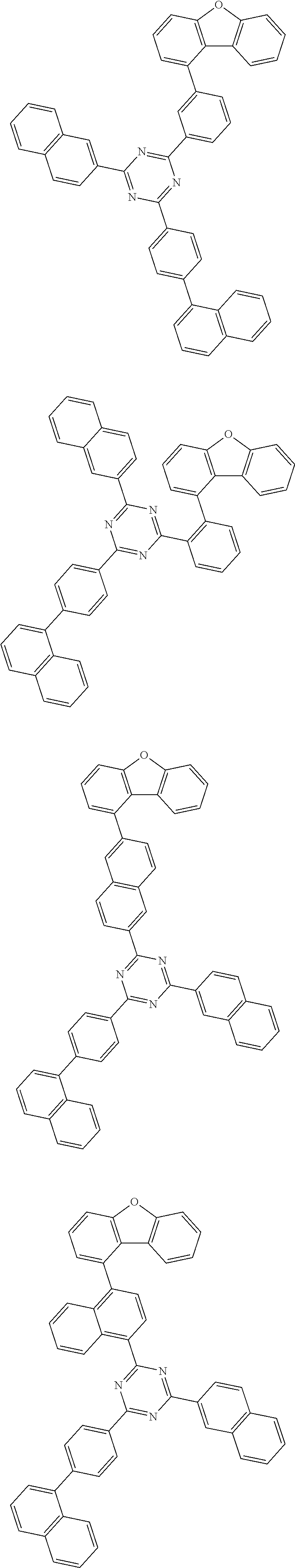 [SMILES: c1cc(-c2nc(-c3ccc(-c4cccc5ccccc45)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4cccc5ccccc45)cc3)nc(-c3ccc4ccccc4c3)n2)c1 ; c1ccc2cc(-c3nc(-c4ccc(-c5cccc6ccccc56)cc4)nc(-c4ccc5cc(-c6cccc7oc8ccccc8c67)ccc5c4)n3)ccc2c1 ; c1ccc2cc(-c3nc(-c4ccc(-c5cccc6ccccc56)cc4)nc(-c4ccc(-c5cccc6oc7ccccc7c56)c5ccccc45)n3)ccc2c1]

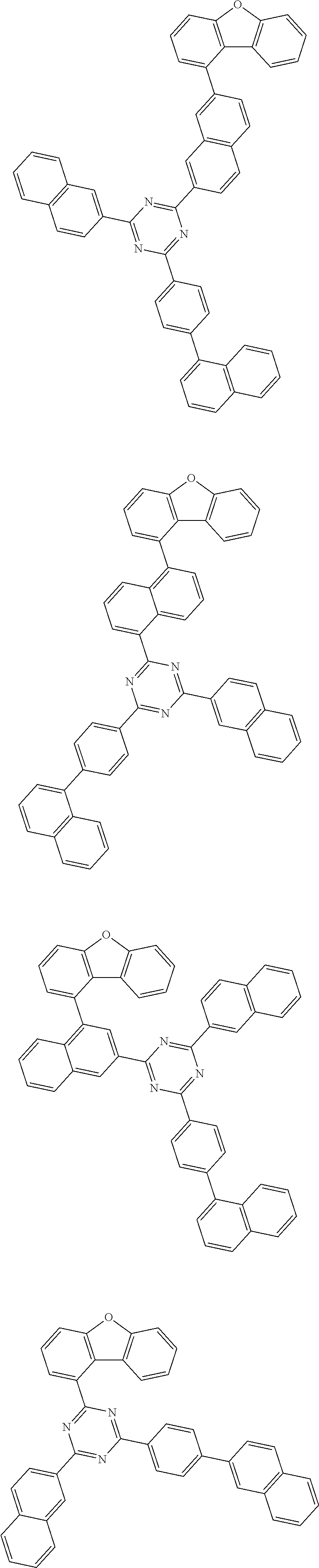 [SMILES: c1ccc2cc(-c3nc(-c4ccc(-c5cccc6ccccc56)cc4)nc(-c4ccc5ccc(-c6cccc7oc8ccccc8c67)cc5c4)n3)ccc2c1 ; c1ccc2cc(-c3nc(-c4ccc(-c5cccc6ccccc56)cc4)nc(-c4cccc5c(-c6cccc7oc8ccccc8c67)cccc45)n3)ccc2c1 ; c1ccc2cc(-c3nc(-c4ccc(-c5cccc6ccccc56)cc4)nc(-c4cc(-c5cccc6oc7ccccc7c56)c5ccccc5c4)n3)ccc2c1 ; c1ccc2cc(-c3ccc(-c4nc(-c5ccc6ccccc6c5)nc(-c5cccc6oc7ccccc7c56)n4)cc3)ccc2c1]

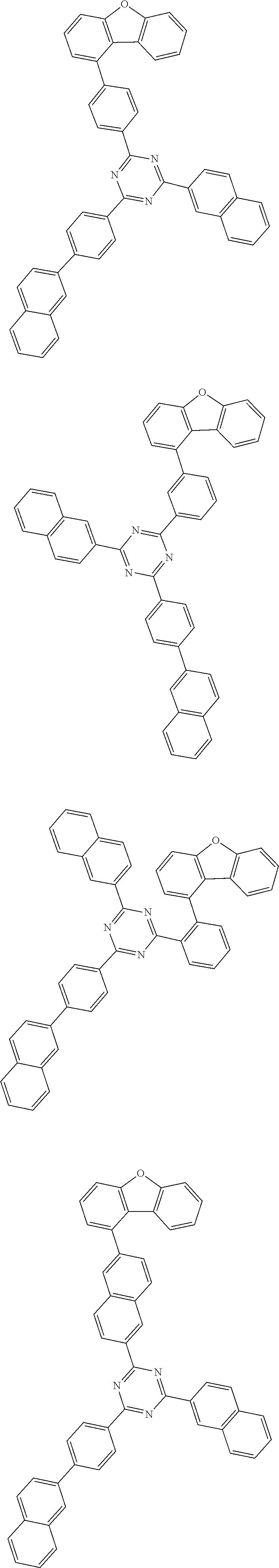 [SMILES: c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3ccc4ccccc4c3)n2)c1 ; c1ccc2cc(-c3ccc(-c4nc(-c5ccc6ccccc6c5)nc(-c5ccc6cc(-c7cccc8oc9ccccc9c78)ccc6c5)n4)cc3)ccc2c1]

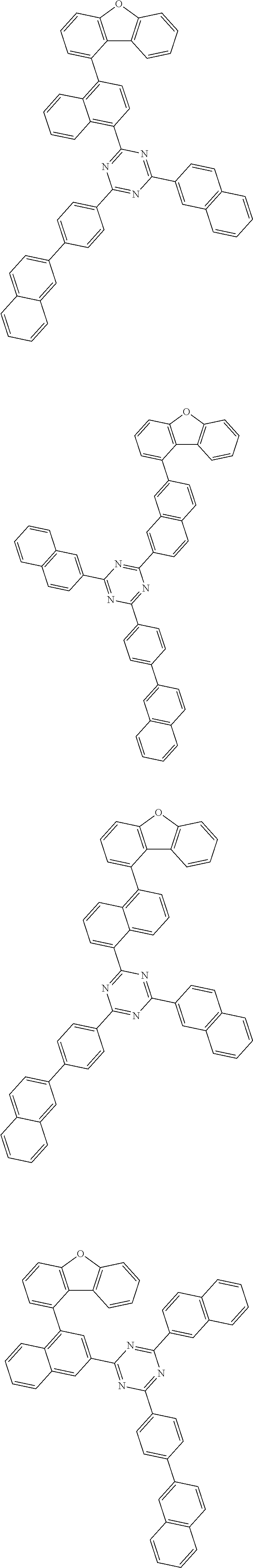 [SMILES: c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1 ; c1ccc2cc(-c3ccc(-c4nc(-c5ccc6ccccc6c5)nc(-c5cccc6c(-c7cccc8oc9ccccc9c78)cccc56)n4)cc3)ccc2c1 ; c1ccc2cc(-c3ccc(-c4nc(-c5ccc6ccccc6c5)nc(-c5cc(-c6cccc7oc8ccccc8c67)c6ccccc6c5)n4)cc3)ccc2c1]

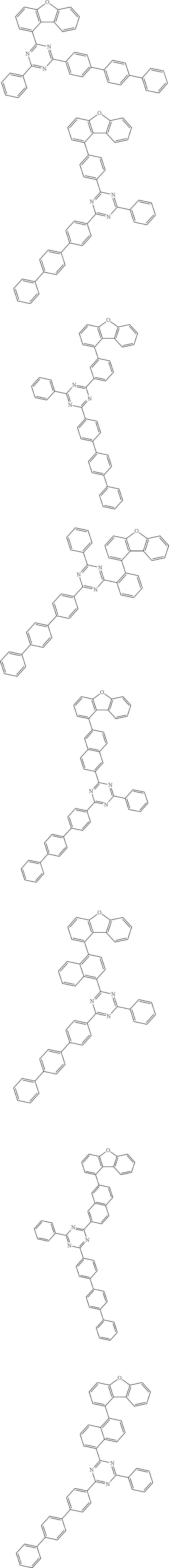

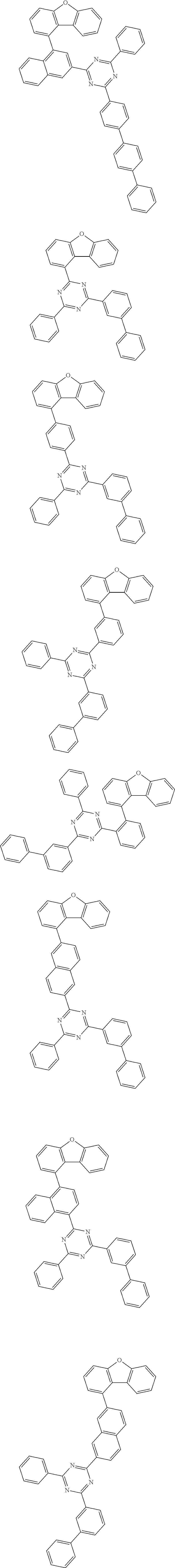

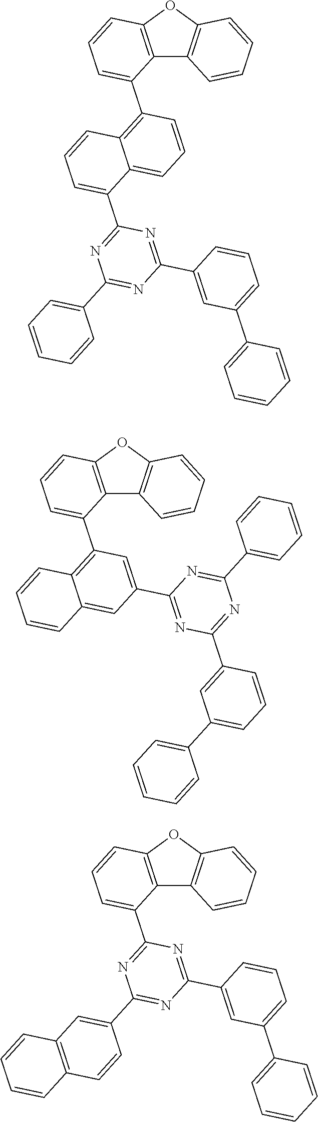

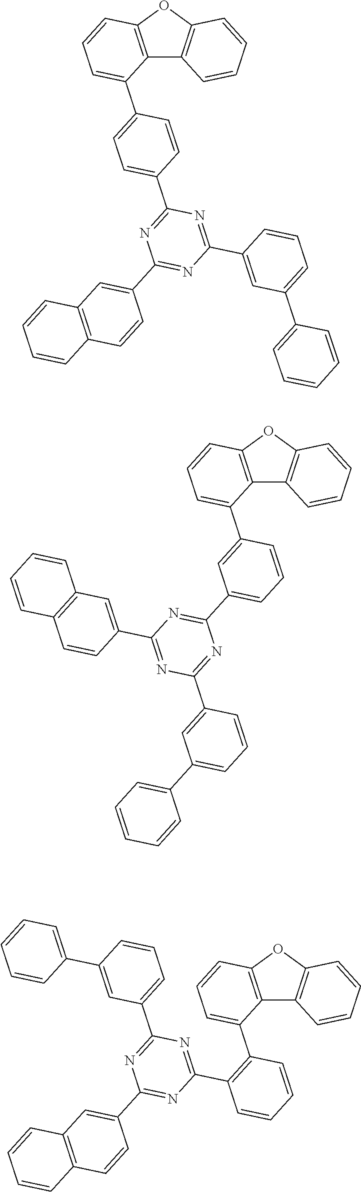 [SMILES: c1ccc(-c2cccc(-c3nc(-c4ccc(-c5cccc6oc7ccccc7c56)cc4)nc(-c4ccc5ccccc5c4)n3)c2)cc1 ; c1ccc(-c2cccc(-c3nc(-c4cccc(-c5cccc6oc7ccccc7c56)c4)nc(-c4ccc5ccccc5c4)n3)c2)cc1 ; c1ccc(-c2cccc(-c3nc(-c4ccc5ccccc5c4)nc(-c4ccccc4-c4cccc5oc6ccccc6c45)n3)c2)cc1]

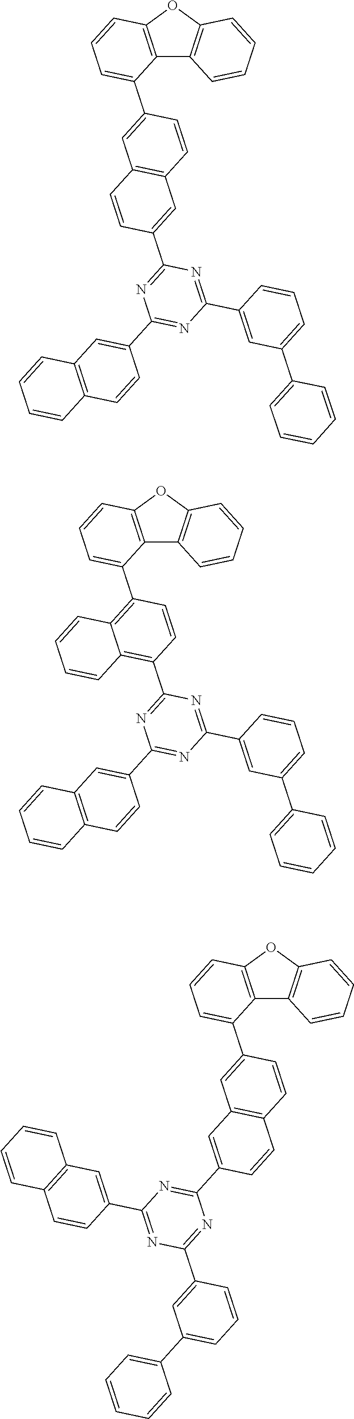 [SMILES: c1ccc(-c2cccc(-c3nc(-c4ccc5ccccc5c4)nc(-c4ccc5cc(-c6cccc7oc8ccccc8c67)ccc5c4)n3)c2)cc1 ; c1ccc(-c2cccc(-c3nc(-c4ccc5ccccc5c4)nc(-c4ccc(-c5cccc6oc7ccccc7c56)c5ccccc45)n3)c2)cc1 ; c1ccc(-c2cccc(-c3nc(-c4ccc5ccccc5c4)nc(-c4ccc5ccc(-c6cccc7oc8ccccc8c67)cc5c4)n3)c2)cc1]

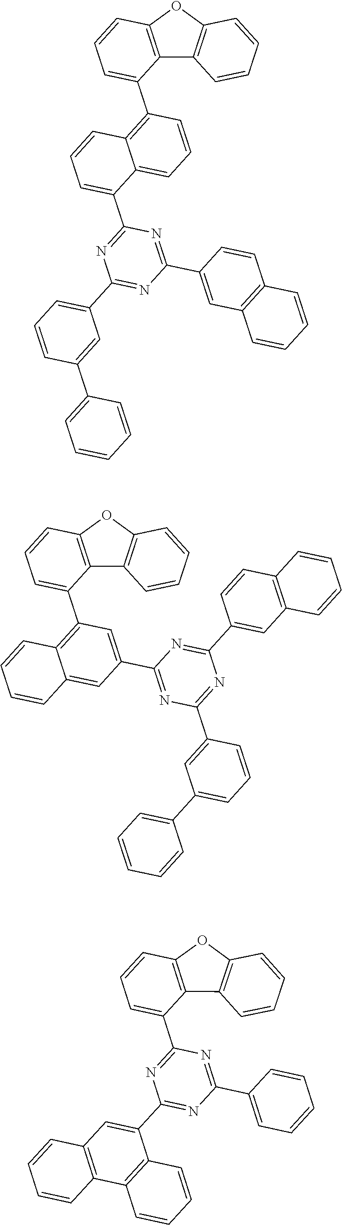 [SMILES: c1ccc(-c2cccc(-c3nc(-c4ccc5ccccc5c4)nc(-c4cccc5c(-c6cccc7oc8ccccc8c67)cccc45)n3)c2)cc1 ; c1ccc(-c2cccc(-c3nc(-c4ccc5ccccc5c4)nc(-c4cc(-c5cccc6oc7ccccc7c56)c5ccccc5c4)n3)c2)cc1 ; c1ccc(-c2nc(-c3cc4ccccc4c4ccccc34)nc(-c3cccc4oc5ccccc5c34)n2)cc1]

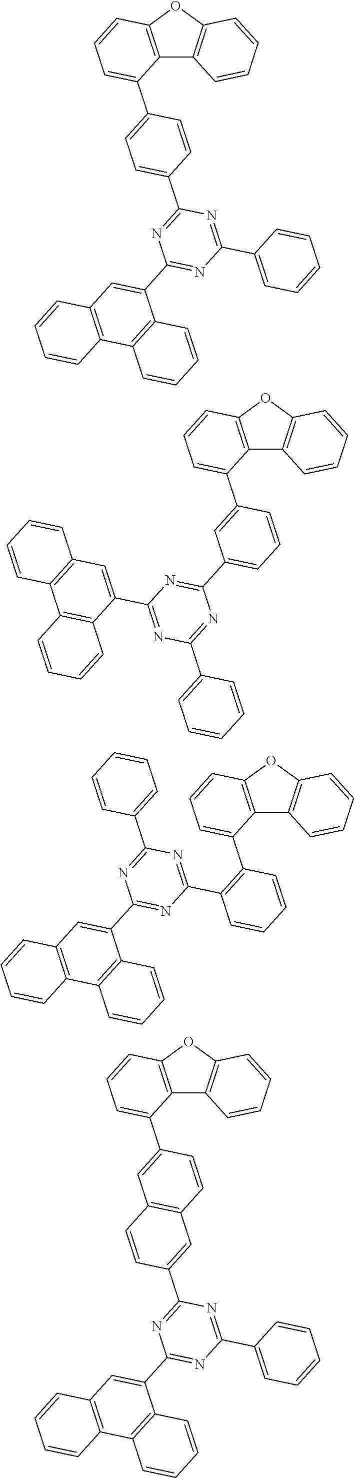

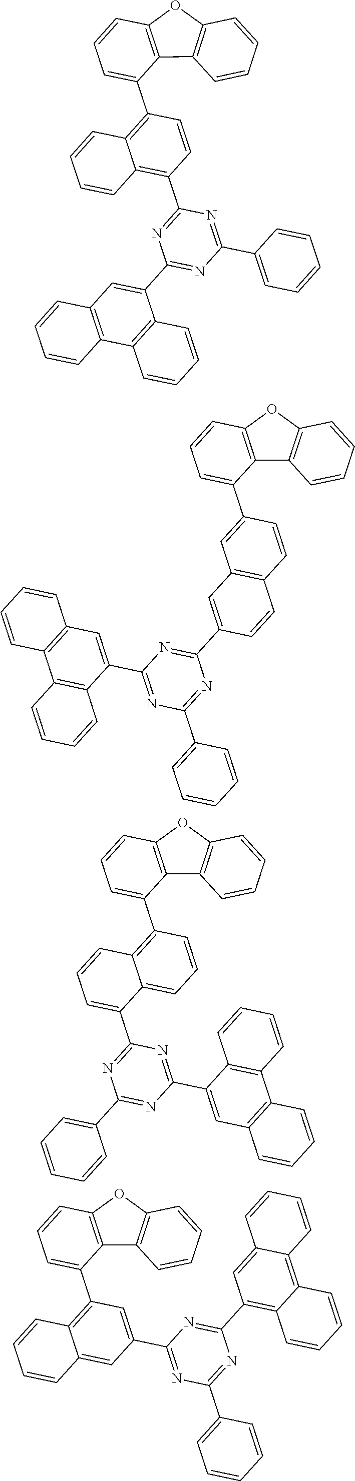

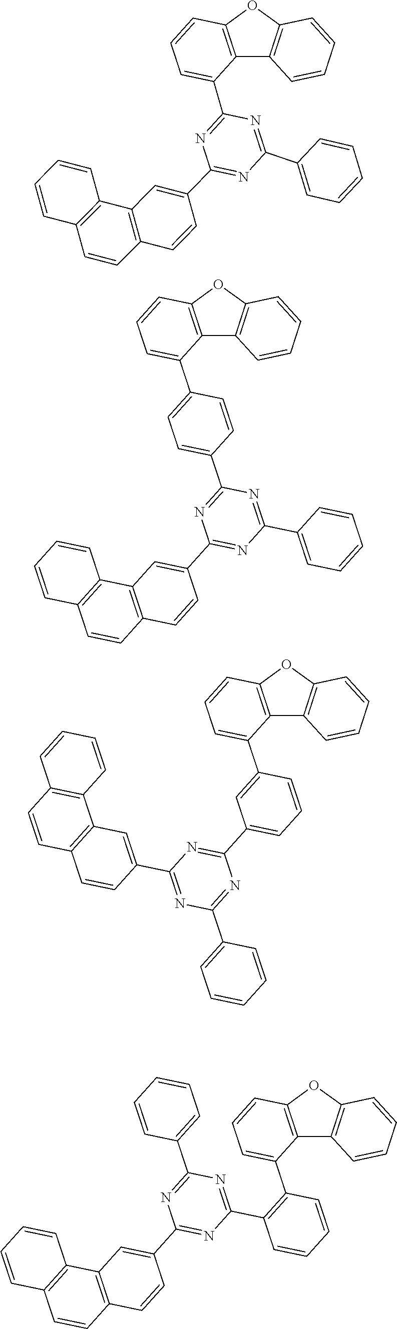 [SMILES: c1ccc(-c2nc(-c3ccc4ccc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4ccc5ccccc5c4c3)n2)cc1 ; c1ccc(-c2nc(-c3cccc(-c4cccc5oc6ccccc6c45)c3)nc(-c3ccc4ccc5ccccc5c4c3)n2)cc1 ; c1ccc(-c2nc(-c3ccc4ccc5ccccc5c4c3)nc(-c3ccccc3-c3cccc4oc5ccccc5c34)n2)cc1]

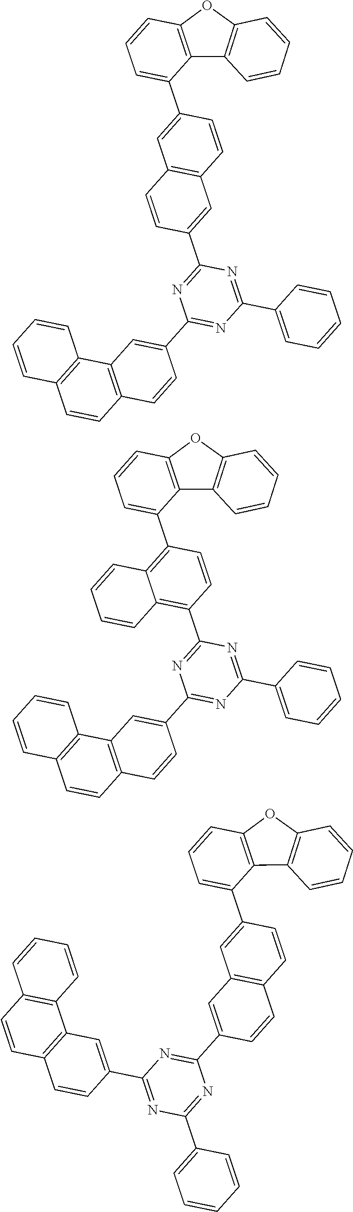 [SMILES: c1ccc(-c2nc(-c3ccc4cc(-c5cccc6oc7ccccc7c56)ccc4c3)nc(-c3ccc4ccc5ccccc5c4c3)n2)cc1 ; c1ccc(-c2nc(-c3ccc4ccc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4ccc5ccccc5c4c3)n2)cc1]

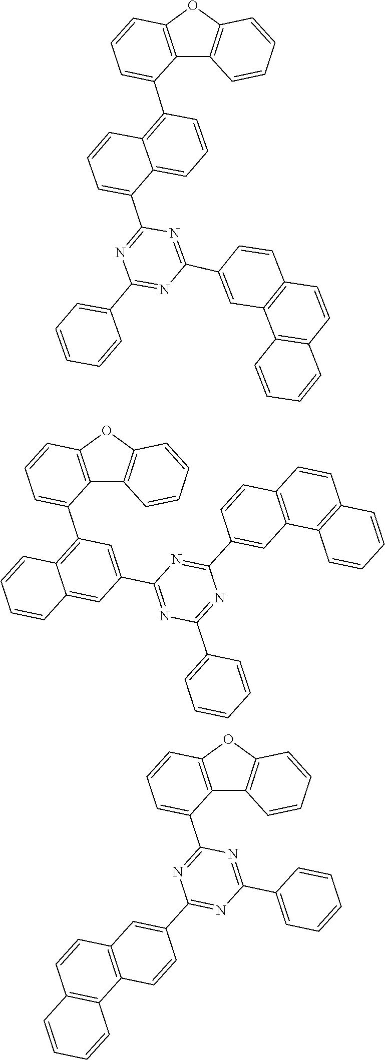

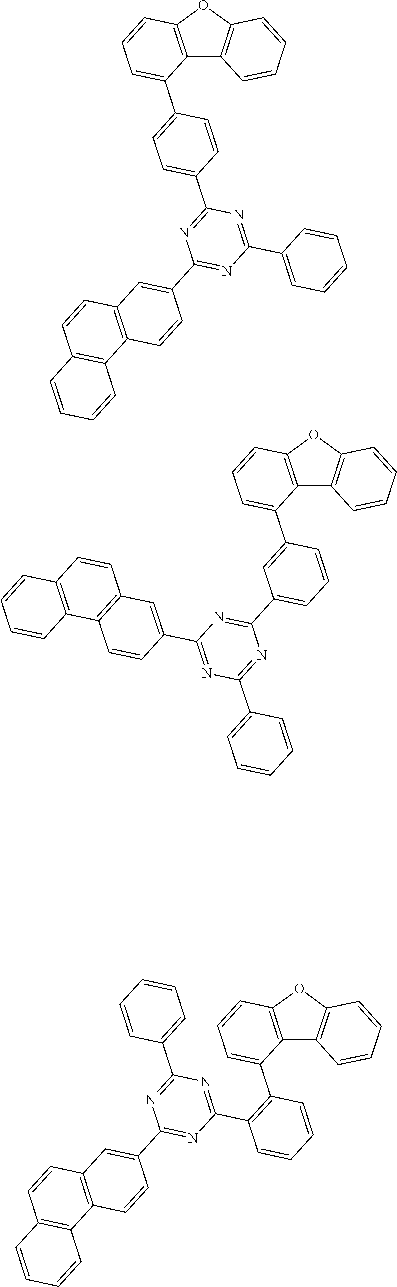 [SMILES: c1ccc(-c2nc(-c3ccc(-c4cccc5oc6ccccc6c45)cc3)nc(-c3ccc4c(ccc5ccccc54)c3)n2)cc1 ; c1ccc(-c2nc(-c3cccc(-c4cccc5oc6ccccc6c45)c3)nc(-c3ccc4c(ccc5ccccc54)c3)n2)cc1 ; c1ccc(-c2nc(-c3ccc4c(ccc5ccccc54)c3)nc(-c3ccccc3-c3cccc4oc5ccccc5c34)n2)cc1]

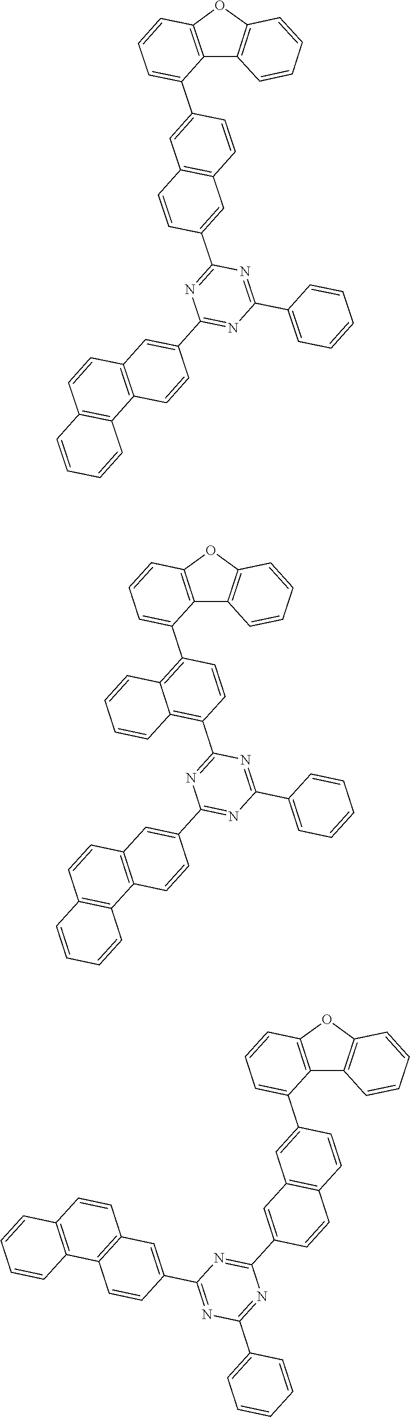 [SMILES: c1ccc(-c2nc(-c3ccc4cc(-c5cccc6oc7ccccc7c56)ccc4c3)nc(-c3ccc4c(ccc5ccccc54)c3)n2)cc1 ; c1ccc(-c2nc(-c3ccc4c(ccc5ccccc54)c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(ccc5ccccc54)c3)n2)cc1]

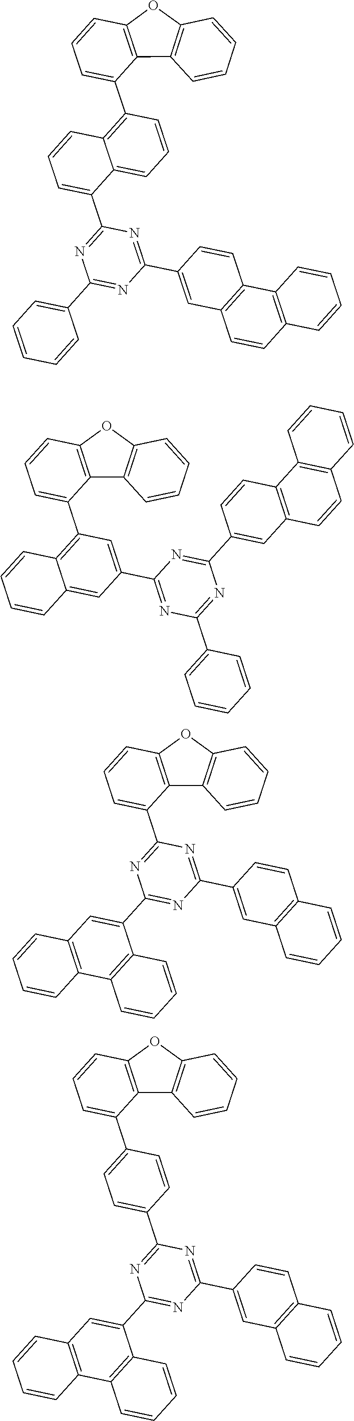

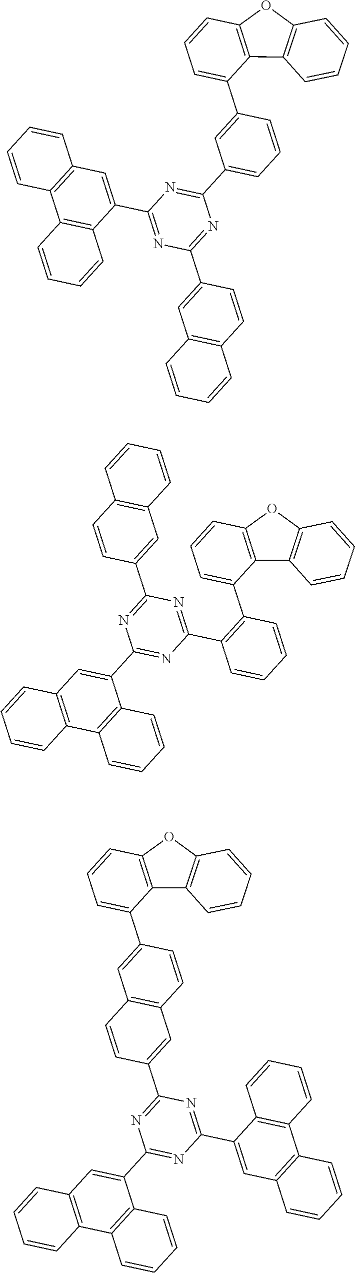 [SMILES: c1cc(-c2nc(-c3ccc4ccccc4c3)nc(-c3cc4ccccc4c4ccccc34)n2)cc(-c2cccc3oc4ccccc4c23)c1 ; c1ccc(-c2cccc3oc4ccccc4c23)c(-c2nc(-c3ccc4ccccc4c3)nc(-c3cc4ccccc4c4ccccc34)n2)c1 ; c1ccc2c(c1)cc(-c1nc(-c3ccc4cc(-c5cccc6oc7ccccc7c56)ccc4c3)nc(-c3cc4ccccc4c4ccccc34)n1)c1ccccc12]

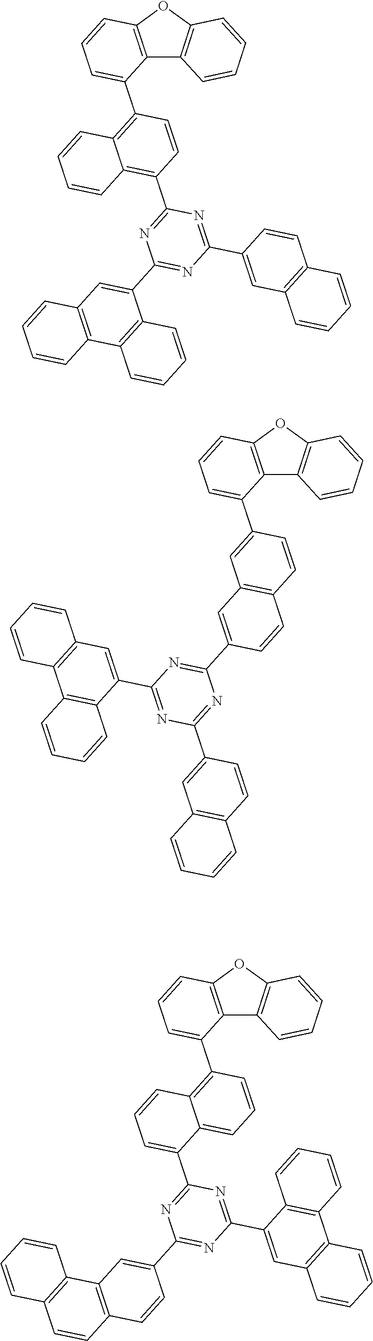 [SMILES: c1ccc2cc(-c3nc(-c4ccc(-c5cccc6oc7ccccc7c56)c5ccccc45)nc(-c4cc5ccccc5c5ccccc45)n3)ccc2c1 ; c1ccc2cc(-c3nc(-c4ccc5ccc(-c6cccc7oc8ccccc8c67)cc5c4)nc(-c4cc5ccccc5c5ccccc45)n3)ccc2c1 ; c1ccc2c(c1)ccc1ccc(-c3nc(-c4cccc5c(-c6cccc7oc8ccccc8c67)cccc45)nc(-c4cc5ccccc5c5ccccc45)n3)cc12]

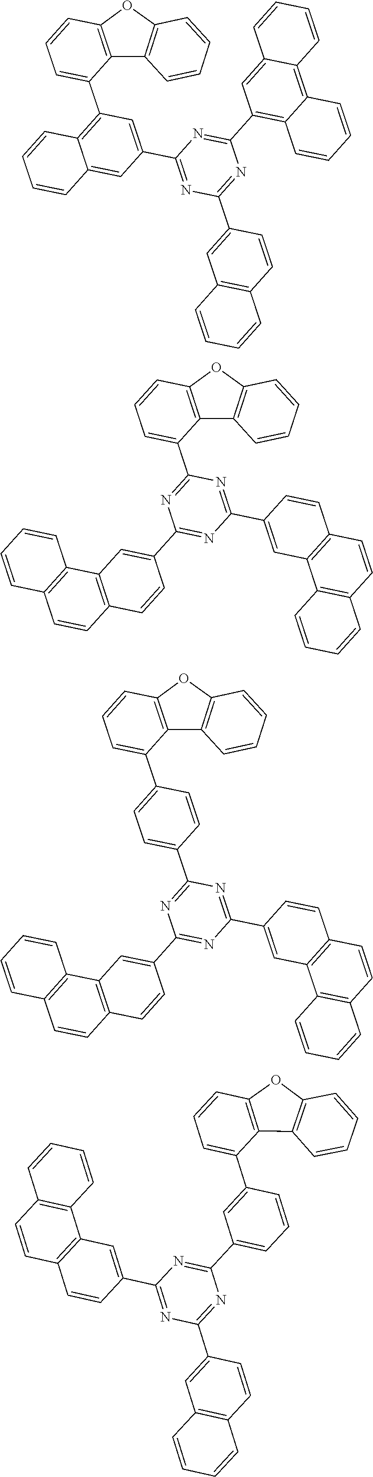

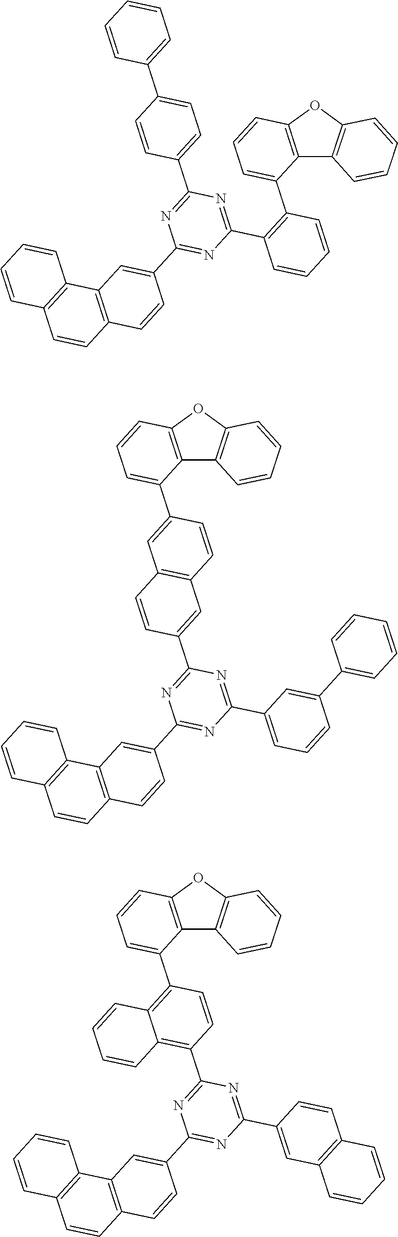 [SMILES: c1ccc(-c2ccc(-c3nc(-c4ccc5ccc6ccccc6c5c4)nc(-c4ccccc4-c4cccc5oc6ccccc6c45)n3)cc2)cc1 ; c1ccc(-c2cccc(-c3nc(-c4ccc5cc(-c6cccc7oc8ccccc8c67)ccc5c4)nc(-c4ccc5ccc6ccccc6c5c4)n3)c2)cc1 ; c1ccc2cc(-c3nc(-c4ccc5ccc6ccccc6c5c4)nc(-c4ccc(-c5cccc6oc7ccccc7c56)c5ccccc45)n3)ccc2c1]

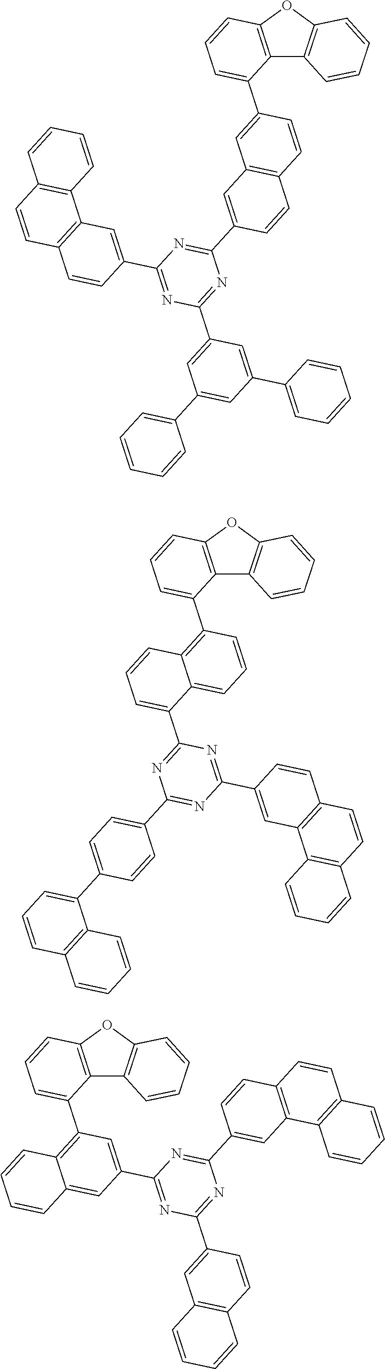

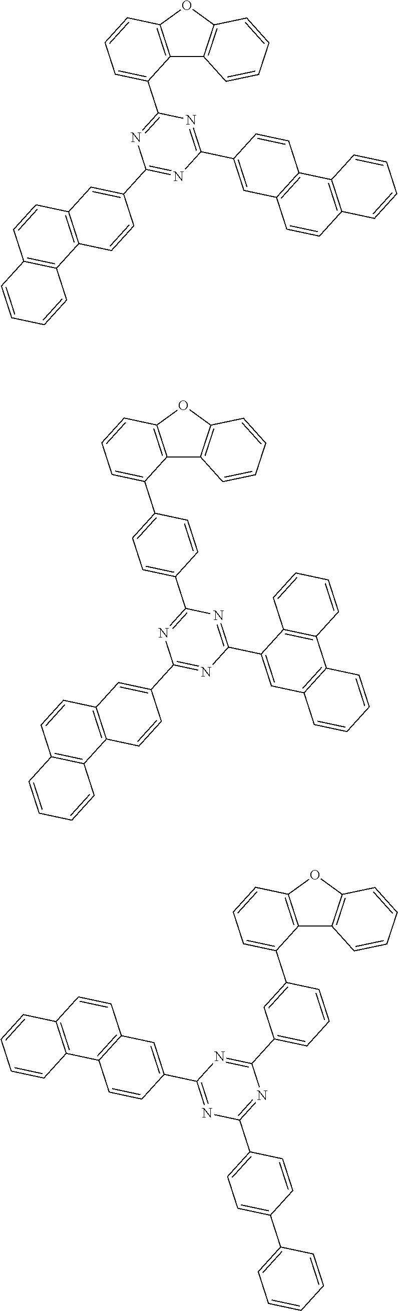 [SMILES: c1ccc2c(c1)ccc1cc(-c3nc(-c4ccc5c(ccc6ccccc65)c4)nc(-c4cccc5oc6ccccc6c45)n3)ccc12 ; c1ccc2c(c1)ccc1cc(-c3nc(-c4ccc(-c5cccc6oc7ccccc7c56)cc4)nc(-c4cc5ccccc5c5ccccc45)n3)ccc12 ; c1ccc(-c2ccc(-c3nc(-c4cccc(-c5cccc6oc7ccccc7c56)c4)nc(-c4ccc5c(ccc6ccccc65)c4)n3)cc2)cc1]

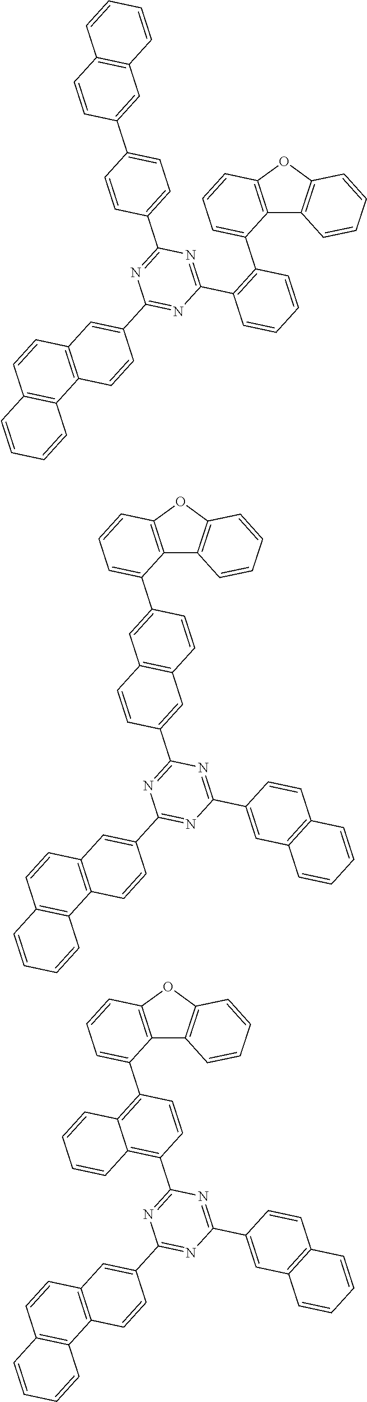 [SMILES: c1ccc(-c2cccc3oc4ccccc4c23)c(-c2nc(-c3ccc(-c4ccc5ccccc5c4)cc3)nc(-c3ccc4c(ccc5ccccc54)c3)n2)c1 ; c1ccc2cc(-c3nc(-c4ccc5cc(-c6cccc7oc8ccccc8c67)ccc5c4)nc(-c4ccc5c(ccc6ccccc65)c4)n3)ccc2c1 ; c1ccc2cc(-c3nc(-c4ccc5c(ccc6ccccc65)c4)nc(-c4ccc(-c5cccc6oc7ccccc7c56)c5ccccc45)n3)ccc2c1]

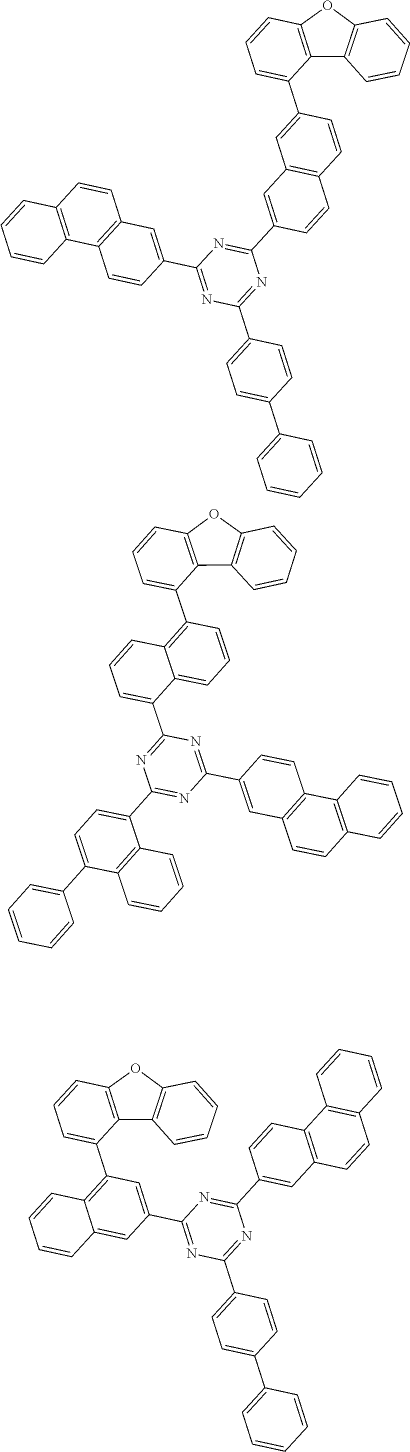

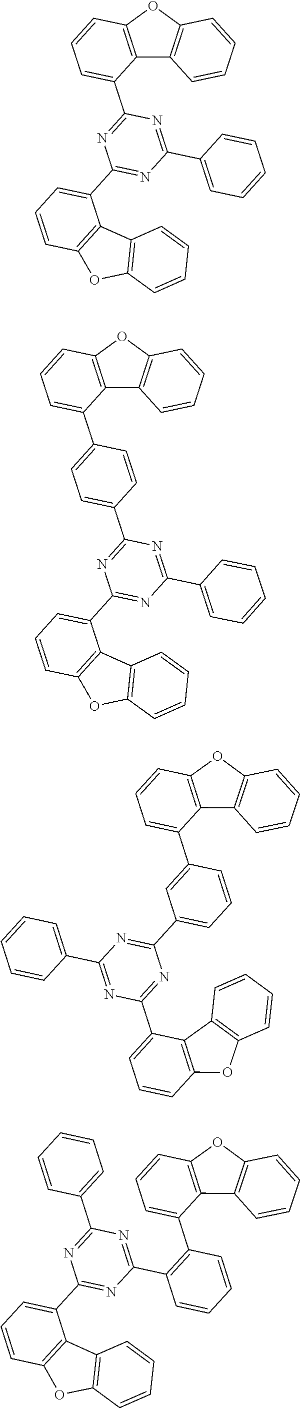 [SMILES: c1ccc(-c2nc(-c3cccc4oc5ccccc5c34)nc(-c3cccc4oc5ccccc5c34)n2)cc1 ; c1ccc(-c2nc(-c3ccc(-c4cccc5oc6ccccc6c45)cc3)nc(-c3cccc4oc5ccccc5c34)n2)cc1 ; c1ccc(-c2nc(-c3cccc(-c4cccc5oc6ccccc6c45)c3)nc(-c3cccc4oc5ccccc5c34)n2)cc1 ; c1ccc(-c2nc(-c3ccccc3-c3cccc4oc5ccccc5c34)nc(-c3cccc4oc5ccccc5c34)n2)cc1]

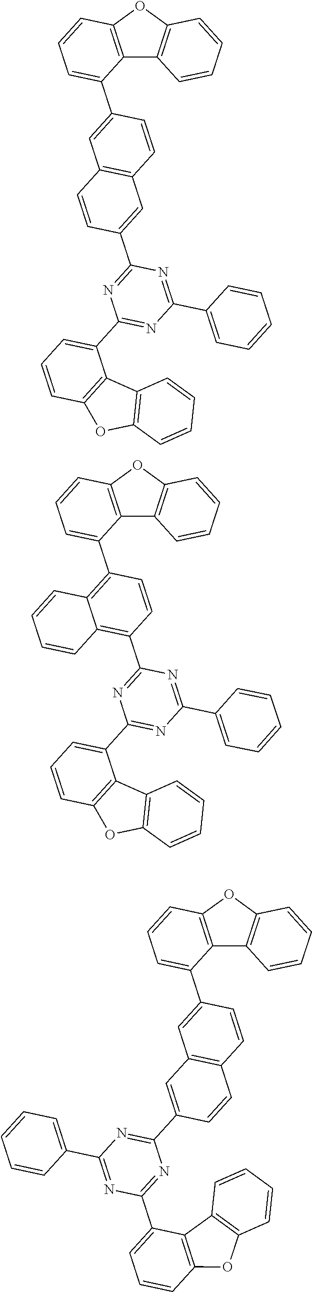

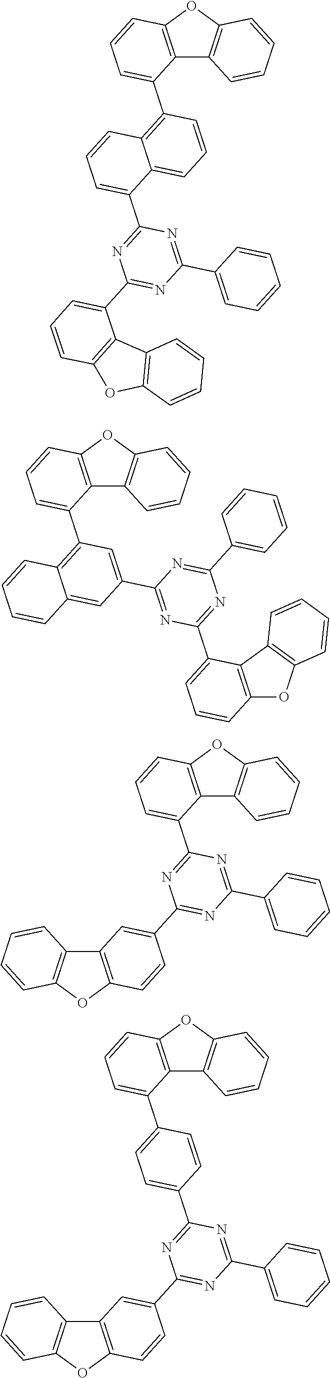

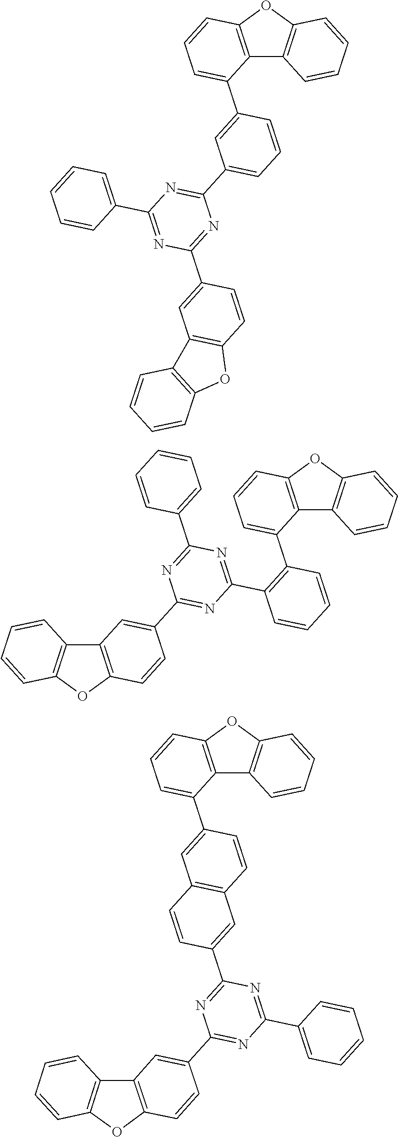

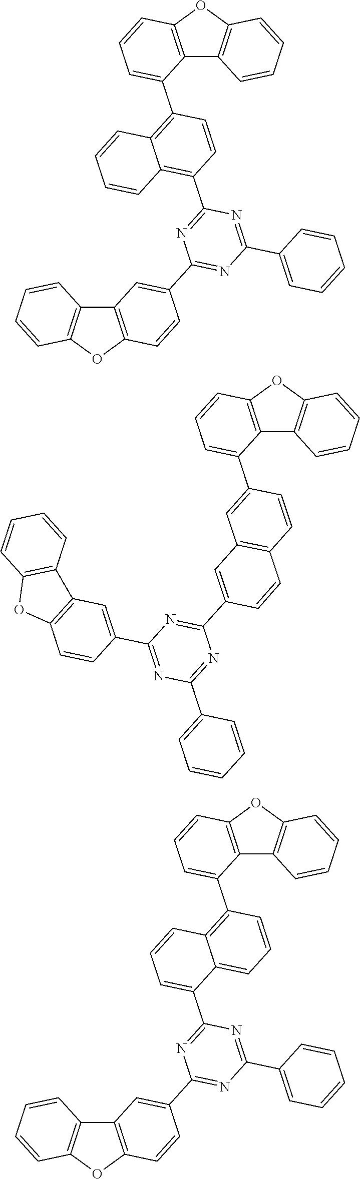

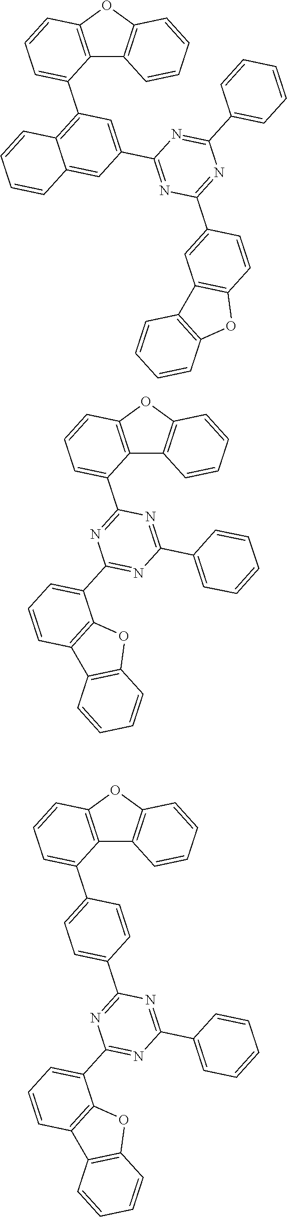

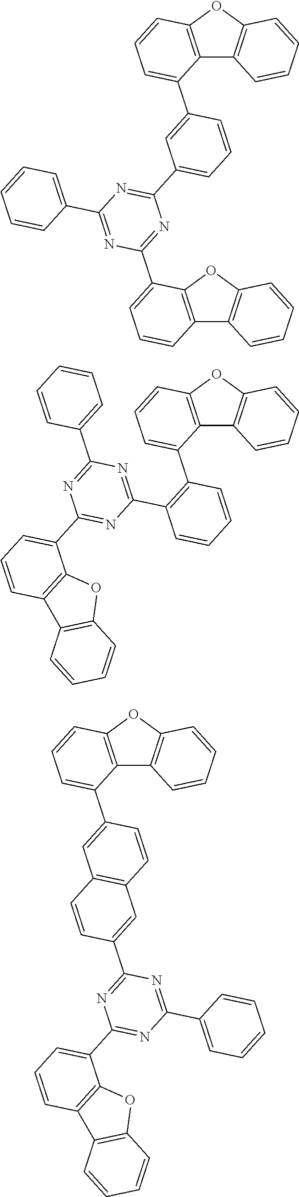

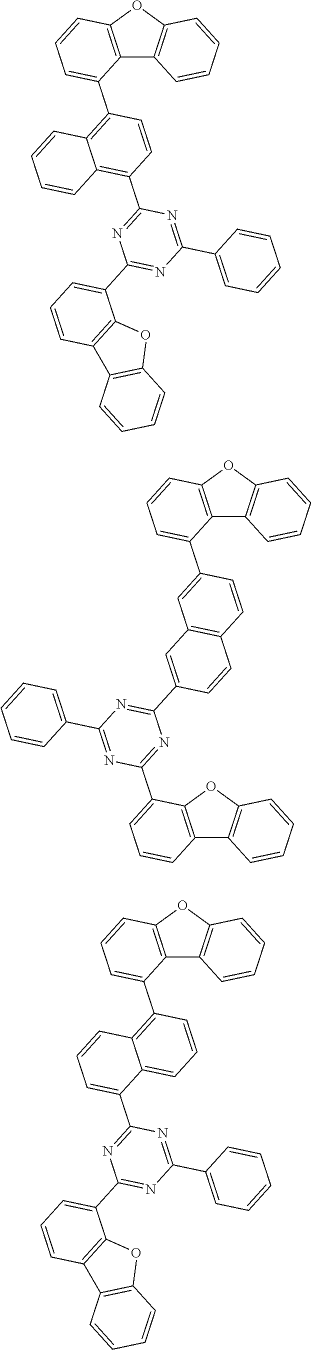

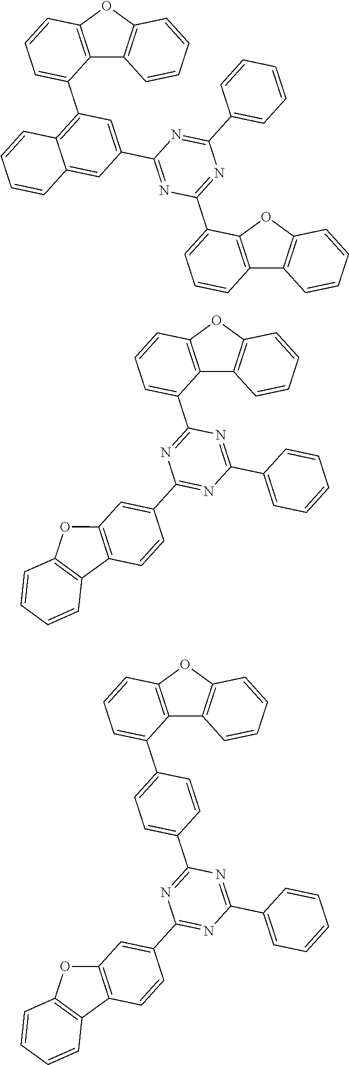

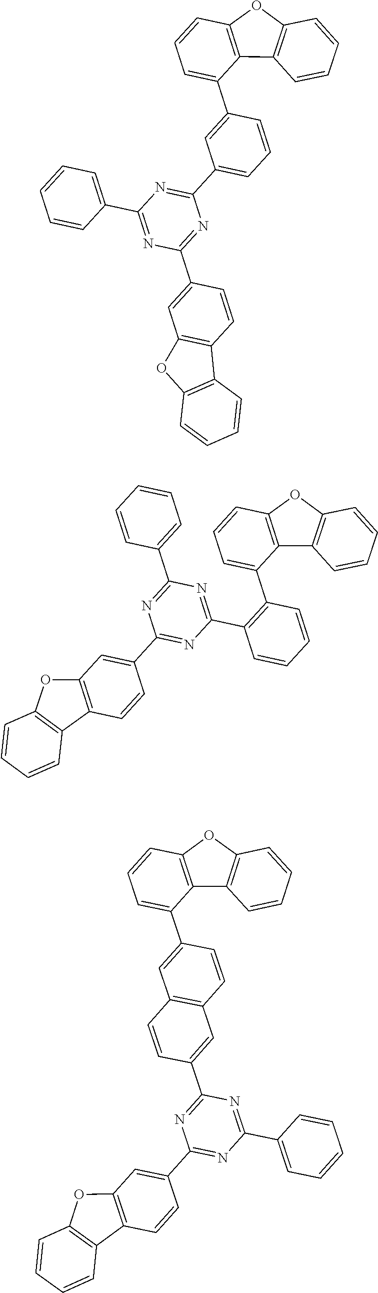 [SMILES: c1ccc(-c2nc(-c3cccc(-c4cccc5oc6ccccc6c45)c3)nc(-c3ccc4c(c3)oc3ccccc34)n2)cc1 ; c1ccc(-c2nc(-c3ccc4c(c3)oc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)oc3ccccc34)n2)cc1]

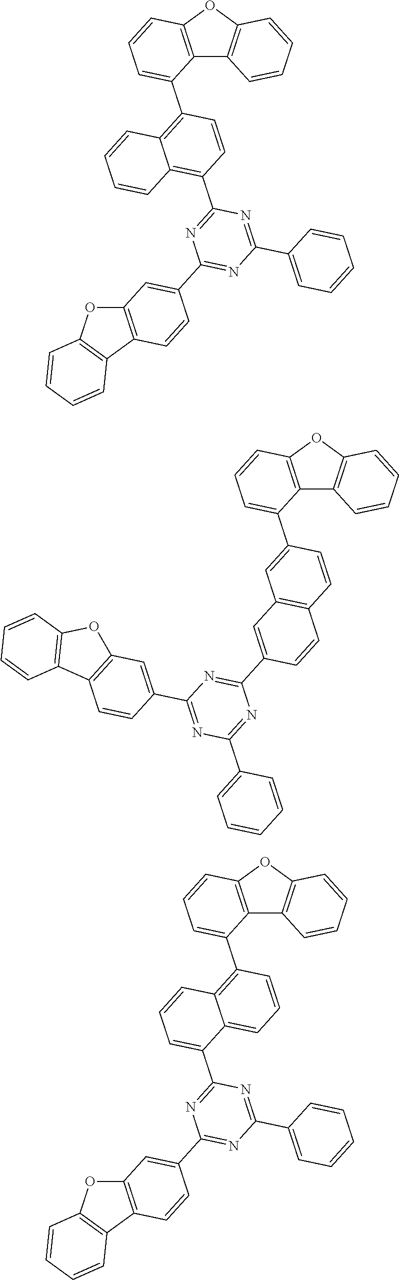

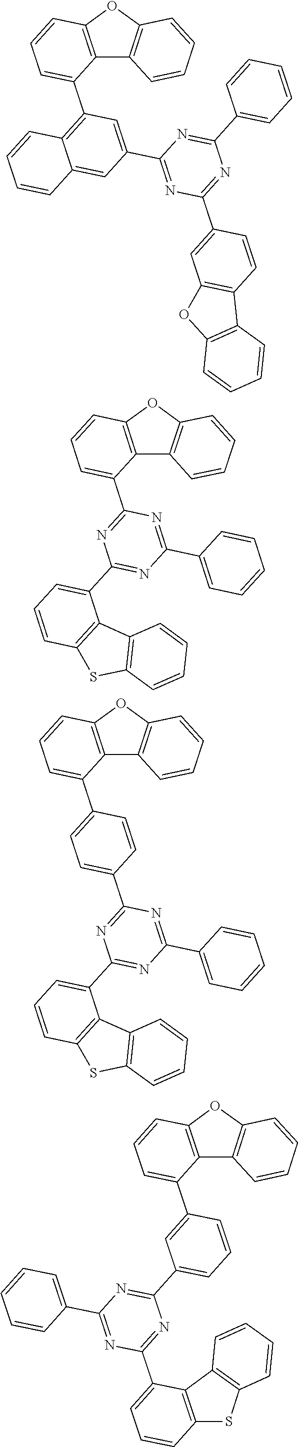

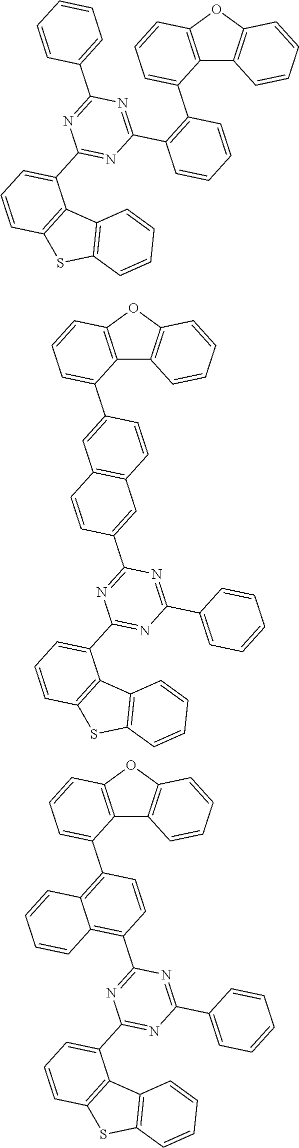

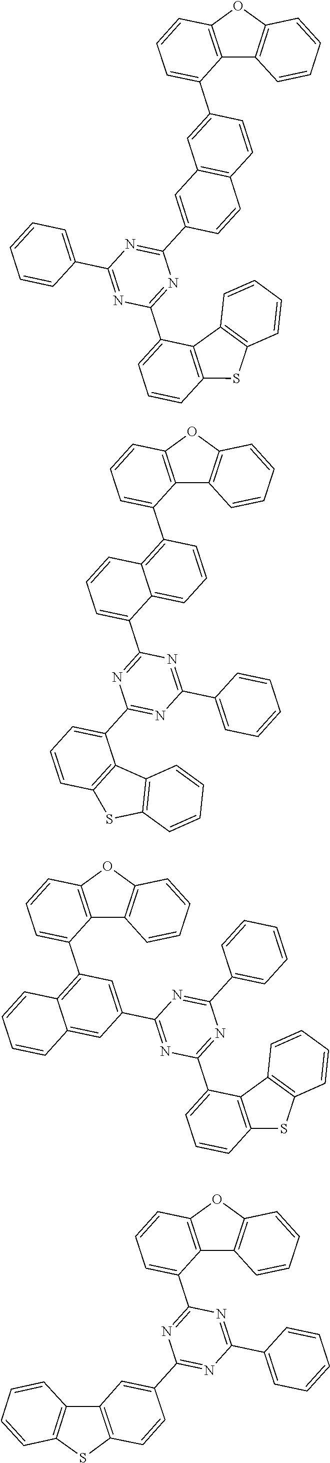

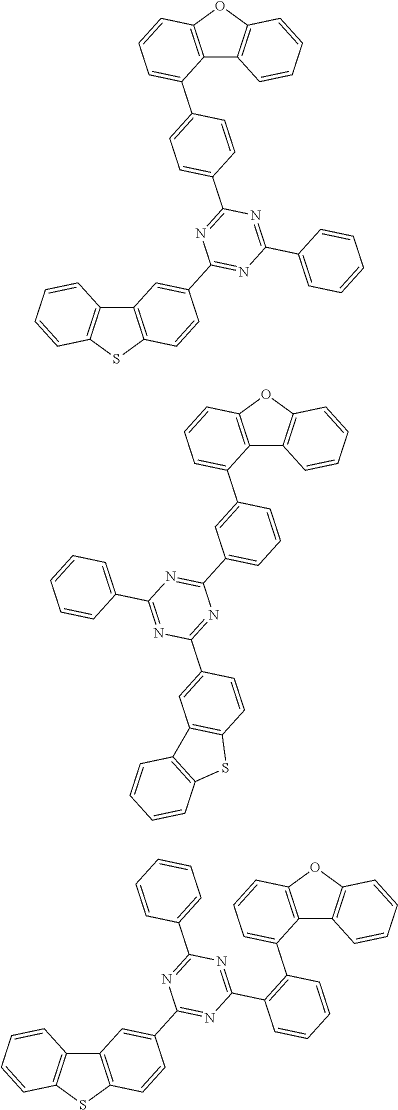

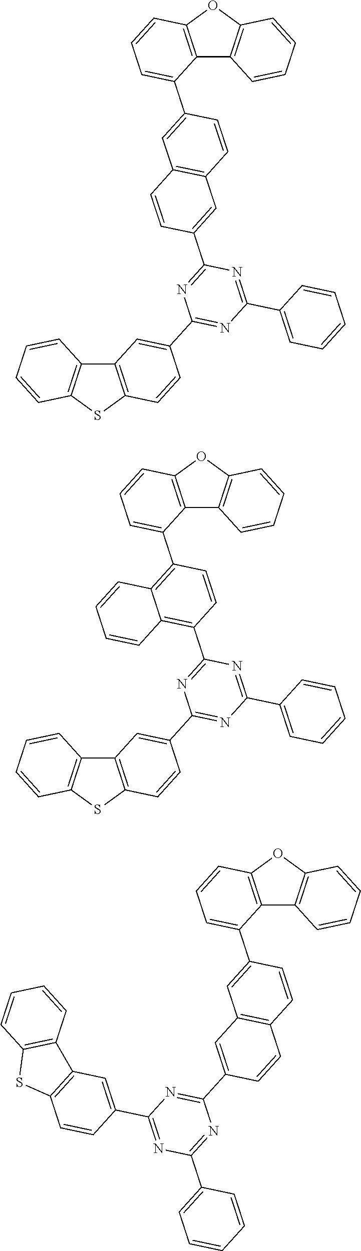 [SMILES: c1ccc(-c2nc(-c3ccc4cc(-c5cccc6oc7ccccc7c56)ccc4c3)nc(-c3ccc4sc5ccccc5c4c3)n2)cc1 ; c1ccc(-c2nc(-c3ccc4sc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4sc5ccccc5c4c3)n2)cc1]

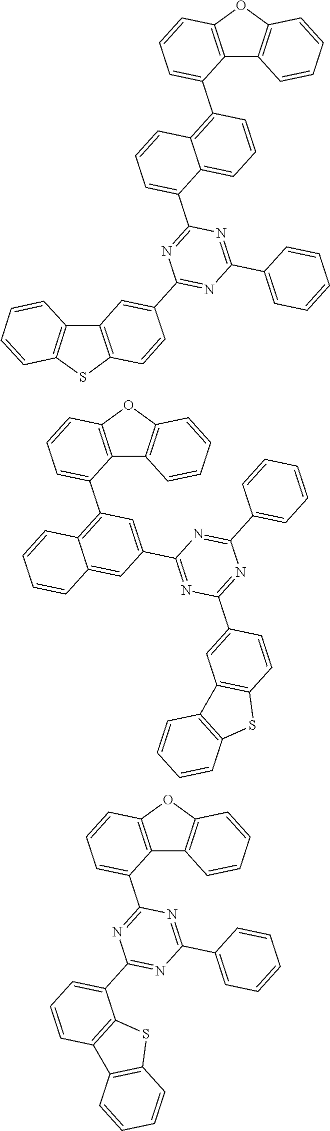

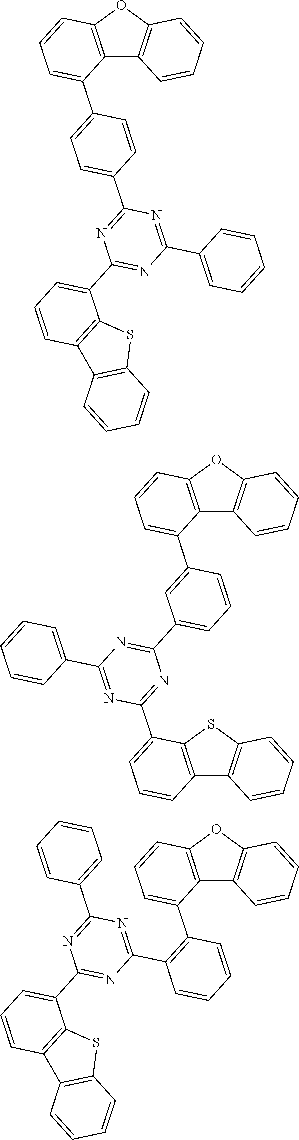

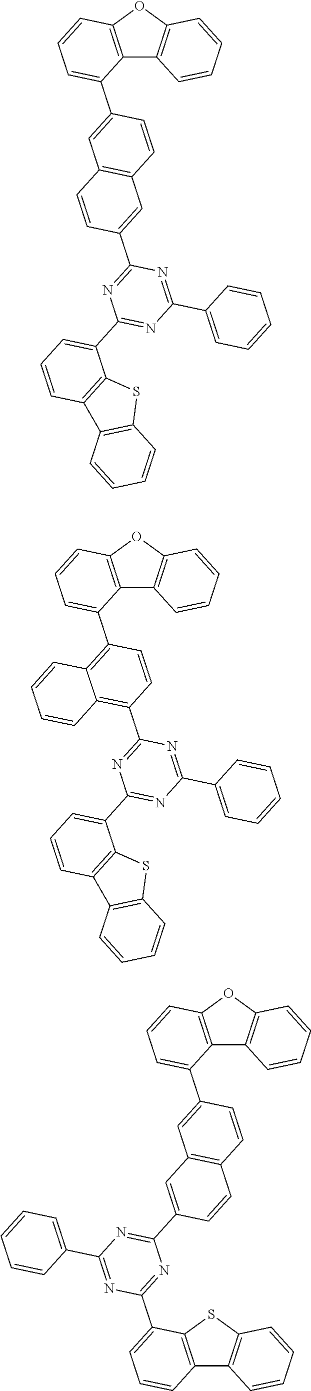 [SMILES: c1ccc(-c2nc(-c3ccc4cc(-c5cccc6oc7ccccc7c56)ccc4c3)nc(-c3cccc4c3sc3ccccc34)n2)cc1 ; c1ccc(-c2nc(-c3ccc(-c4cccc5oc6ccccc6c45)c4ccccc34)nc(-c3cccc4c3sc3ccccc34)n2)cc1 ; c1ccc(-c2nc(-c3ccc4ccc(-c5cccc6oc7ccccc7c56)cc4c3)nc(-c3cccc4c3sc3ccccc34)n2)cc1]

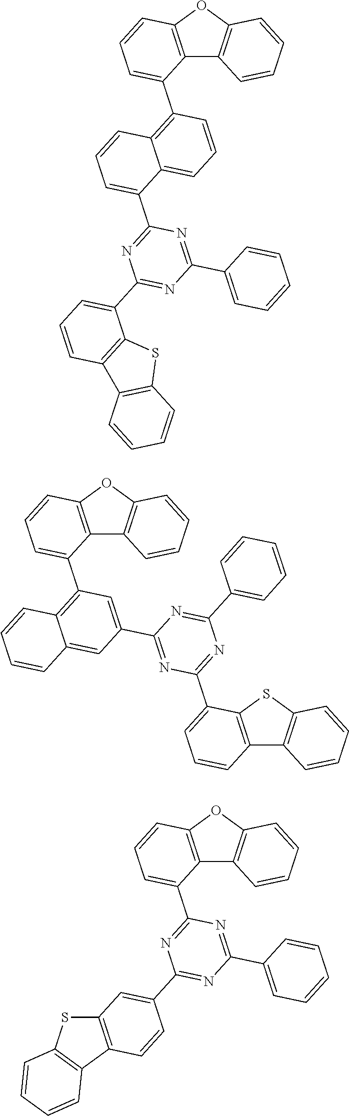

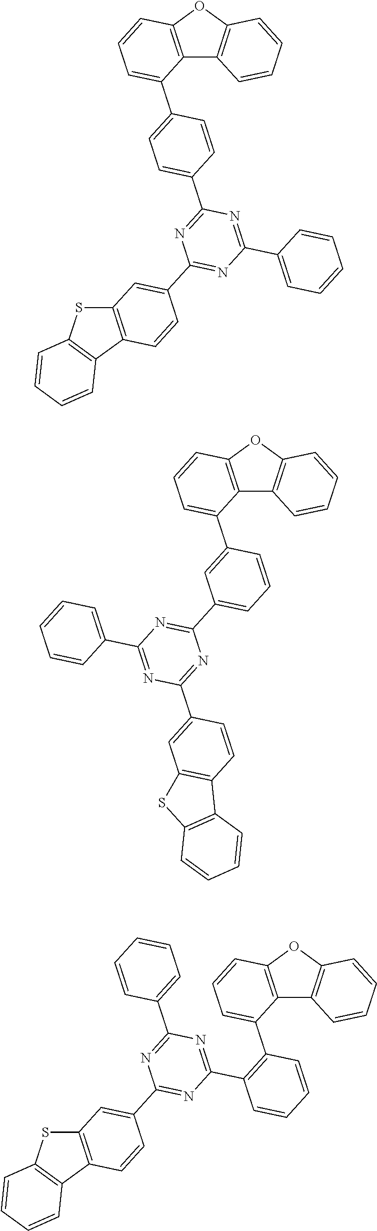 [SMILES: c1ccc(-c2nc(-c3ccc(-c4cccc5oc6ccccc6c45)cc3)nc(-c3ccc4c(c3)sc3ccccc34)n2)cc1 ; c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1]

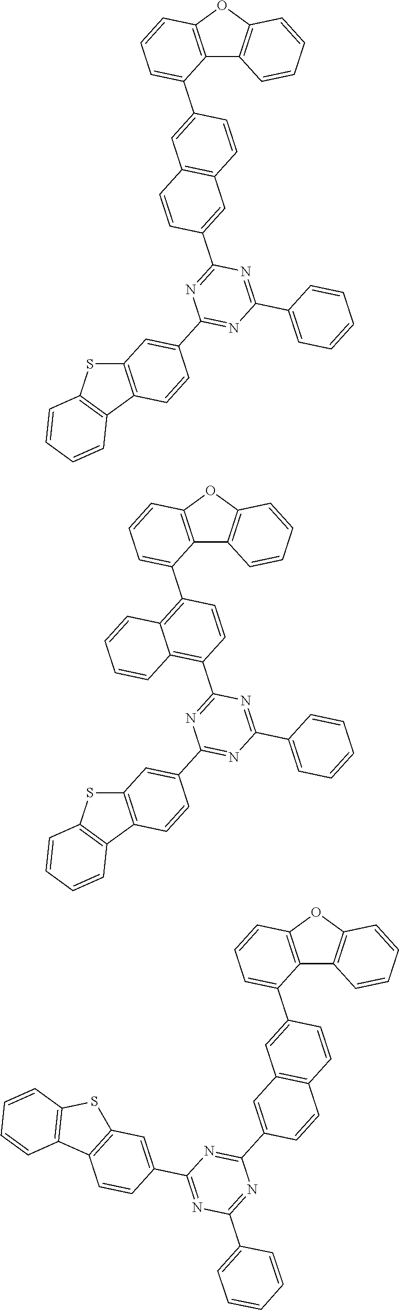 [SMILES: c1ccc(-c2nc(-c3ccc4cc(-c5cccc6oc7ccccc7c56)ccc4c3)nc(-c3ccc4c(c3)sc3ccccc34)n2)cc1 ; c1ccc(-c2nc(-c3ccc4c(c3)sc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)sc3ccccc34)n2)cc1]

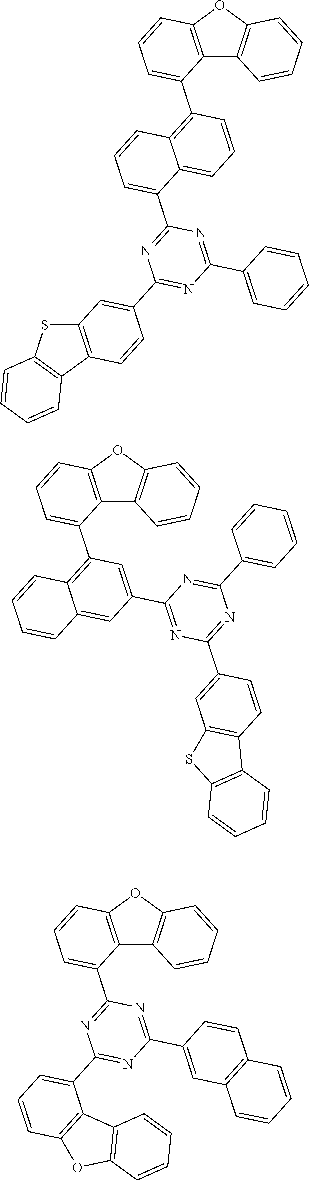 [SMILES: c1ccc(-c2nc(-c3ccc4c(c3)sc3ccccc34)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(c3)sc3ccccc34)n2)cc1 ; c1ccc2cc(-c3nc(-c4cccc5oc6ccccc6c45)nc(-c4cccc5oc6ccccc6c45)n3)ccc2c1]

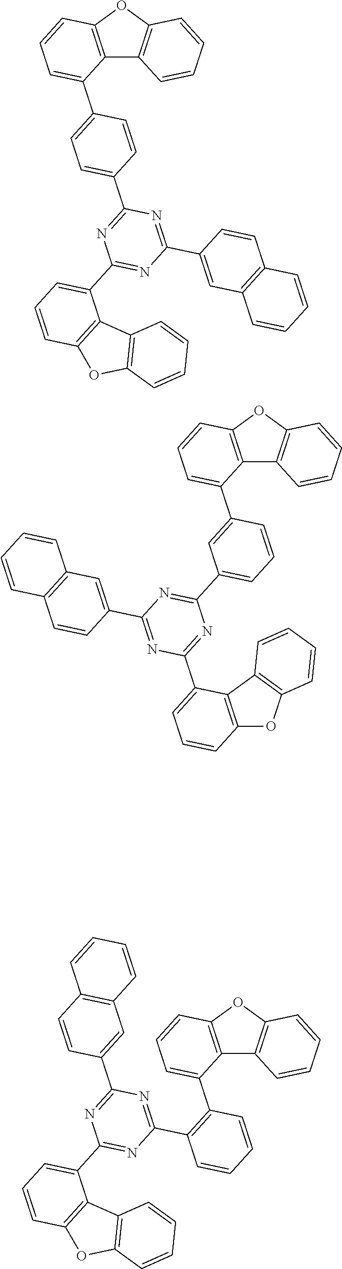 [SMILES: c1ccc2cc(-c3nc(-c4ccc(-c5cccc6oc7ccccc7c56)cc4)nc(-c4cccc5oc6ccccc6c45)n3)ccc2c1 ; c1cc(-c2nc(-c3ccc4ccccc4c3)nc(-c3cccc4oc5ccccc5c34)n2)cc(-c2cccc3oc4ccccc4c23)c1 ; c1ccc(-c2cccc3oc4ccccc4c23)c(-c2nc(-c3ccc4ccccc4c3)nc(-c3cccc4oc5ccccc5c34)n2)c1]

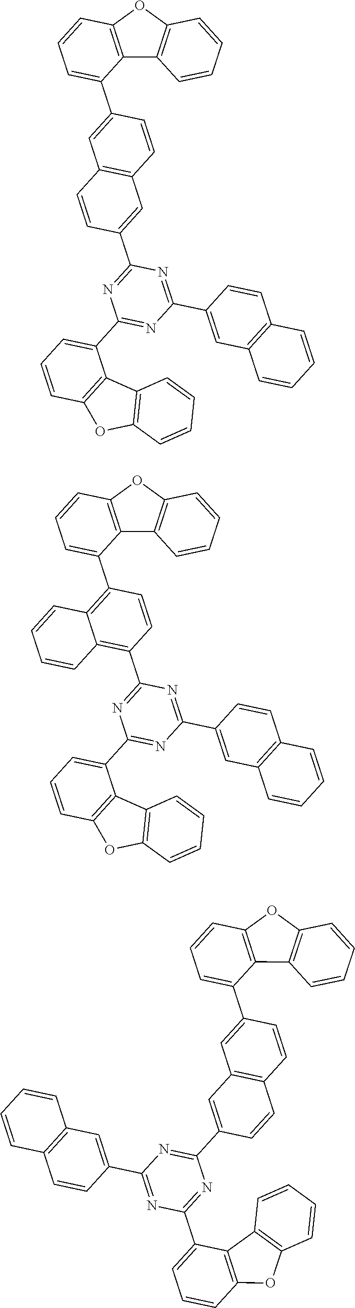

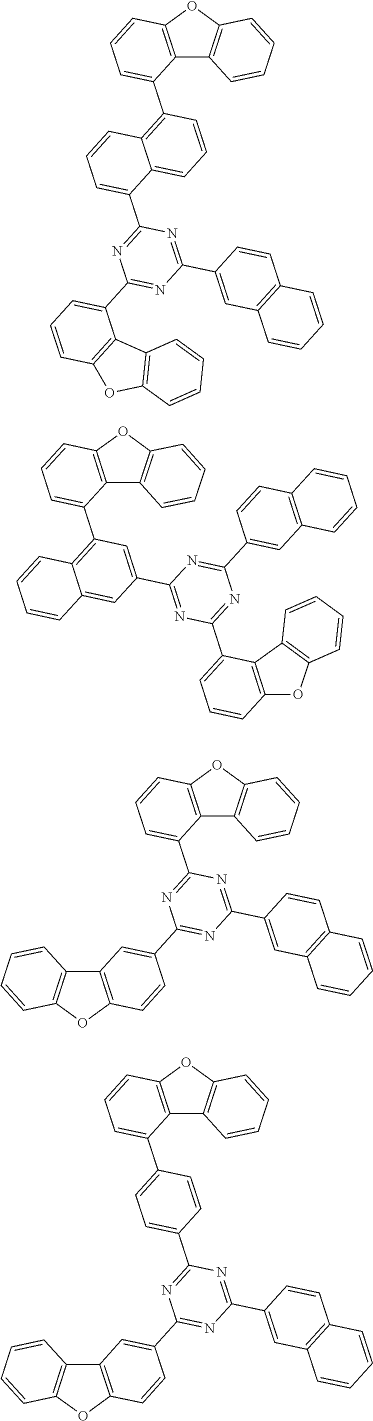

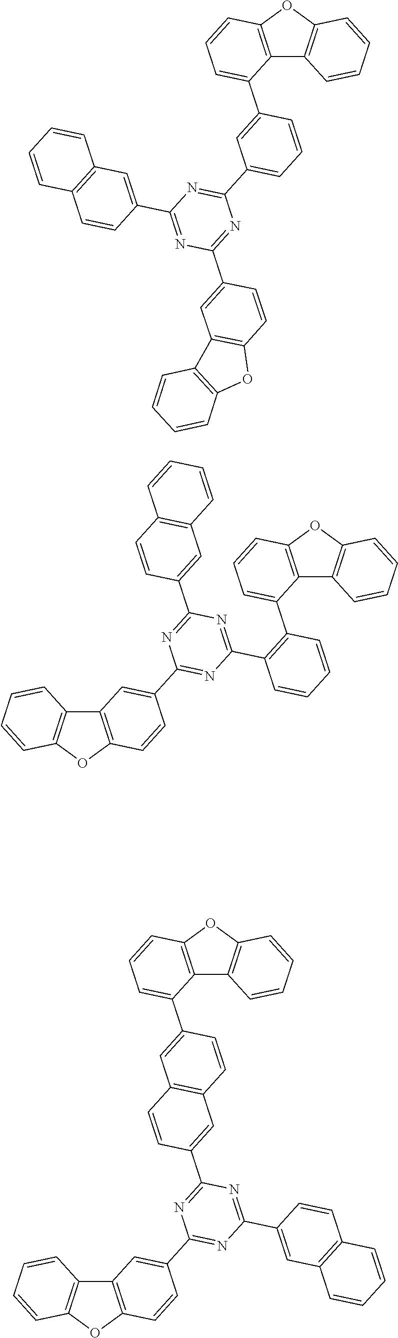

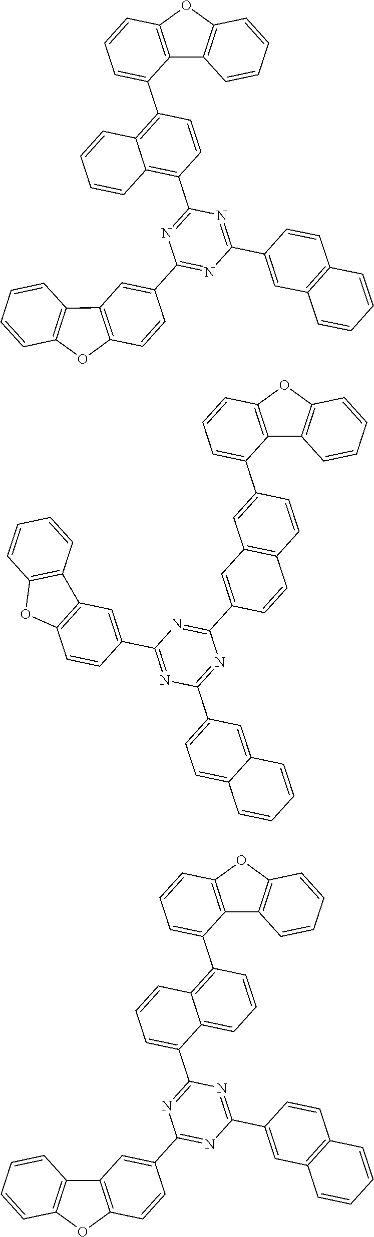

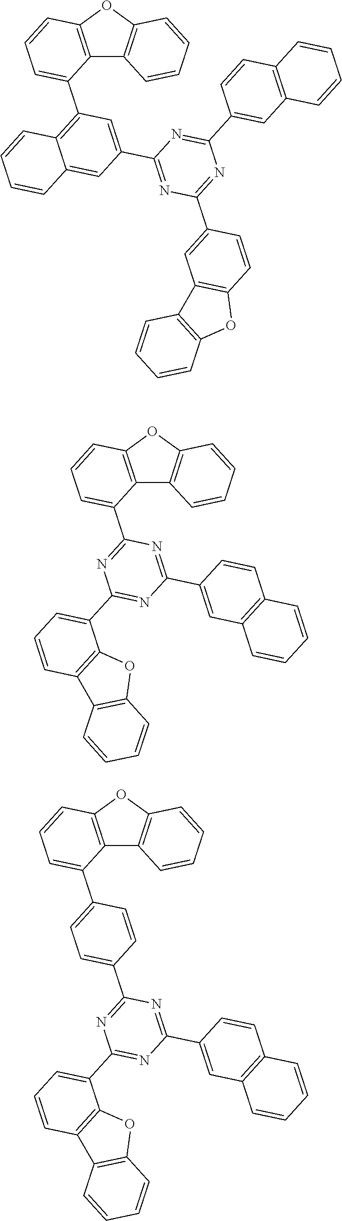

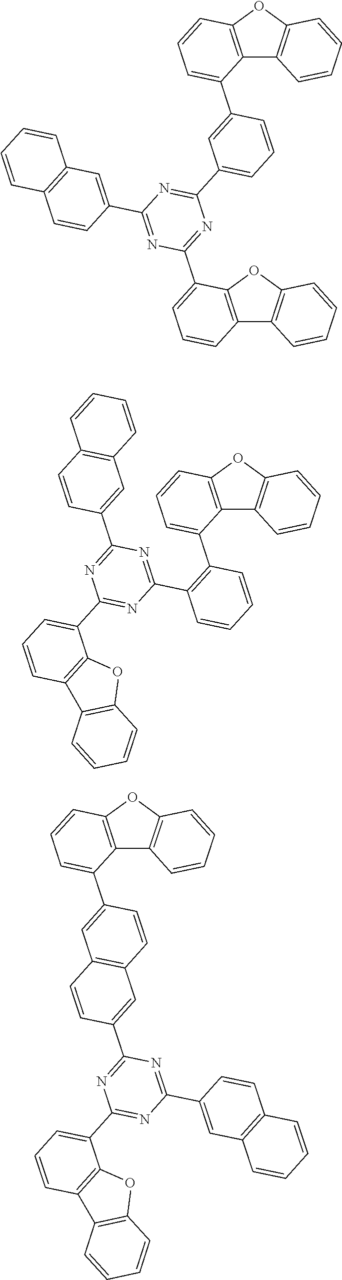

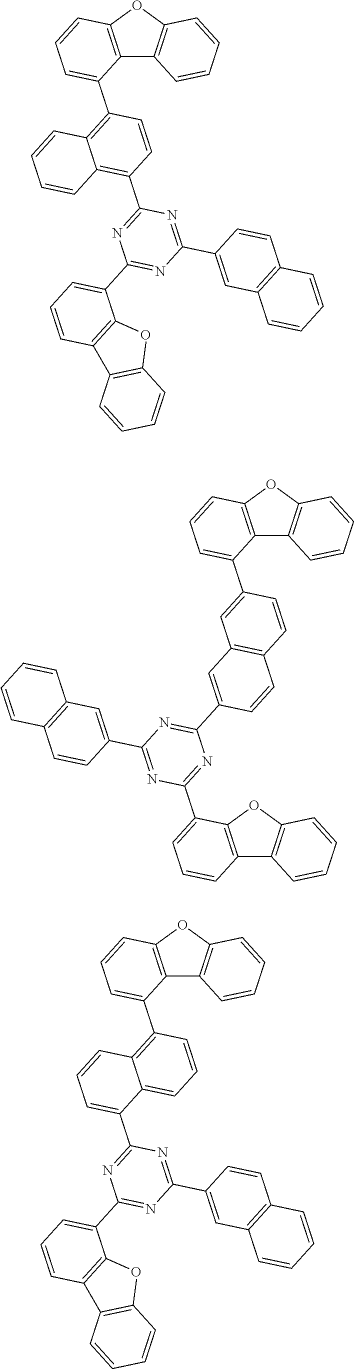 [SMILES: c1ccc2cc(-c3nc(-c4ccc(-c5cccc6oc7ccccc7c56)c5ccccc45)nc(-c4cccc5c4oc4ccccc45)n3)ccc2c1 ; c1ccc2cc(-c3nc(-c4ccc5ccc(-c6cccc7oc8ccccc8c67)cc5c4)nc(-c4cccc5c4oc4ccccc45)n3)ccc2c1 ; c1ccc2cc(-c3nc(-c4cccc5c(-c6cccc7oc8ccccc8c67)cccc45)nc(-c4cccc5c4oc4ccccc45)n3)ccc2c1]

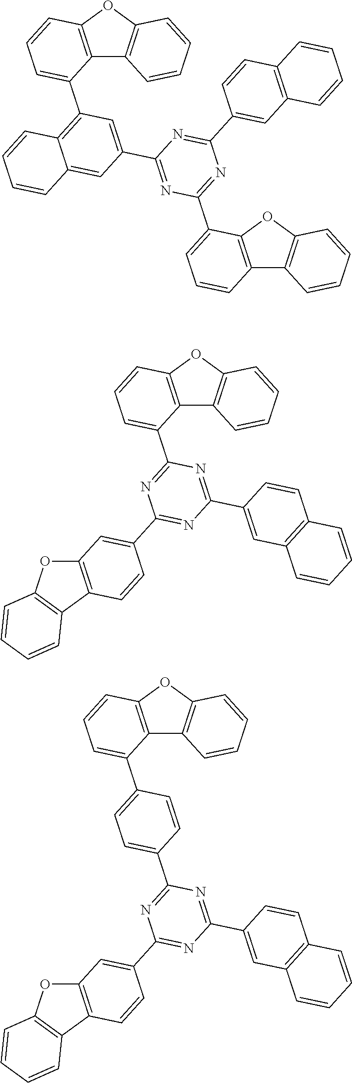 [SMILES: c1ccc2cc(-c3nc(-c4cc(-c5cccc6oc7ccccc7c56)c5ccccc5c4)nc(-c4cccc5c4oc4ccccc45)n3)ccc2c1 ; c1ccc2cc(-c3nc(-c4ccc5c(c4)oc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4ccc5c(c4)oc4ccccc45)n3)ccc2c1]

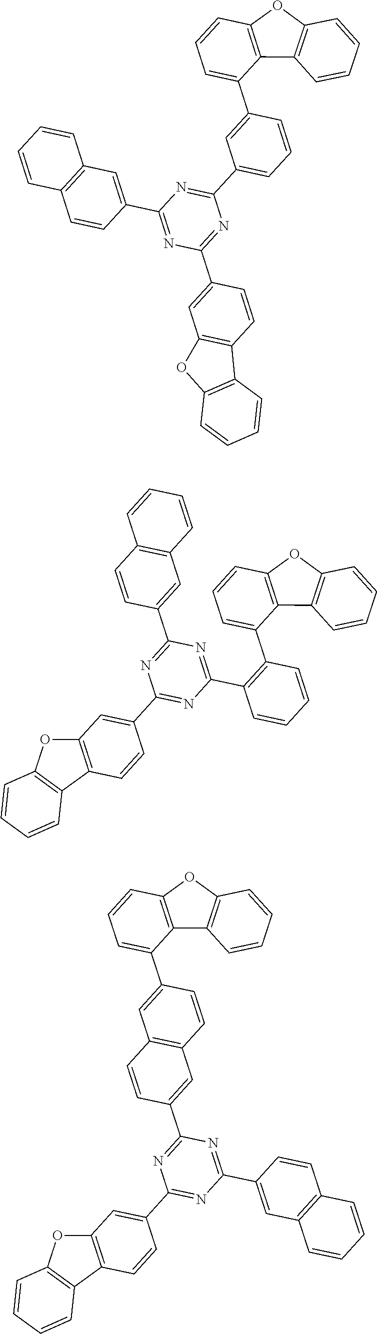 [SMILES: c1cc(-c2nc(-c3ccc4ccccc4c3)nc(-c3ccc4c(c3)oc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)oc3ccccc34)n2)c1 ; c1ccc2cc(-c3nc(-c4ccc5cc(-c6cccc7oc8ccccc8c67)ccc5c4)nc(-c4ccc5c(c4)oc4ccccc45)n3)ccc2c1]

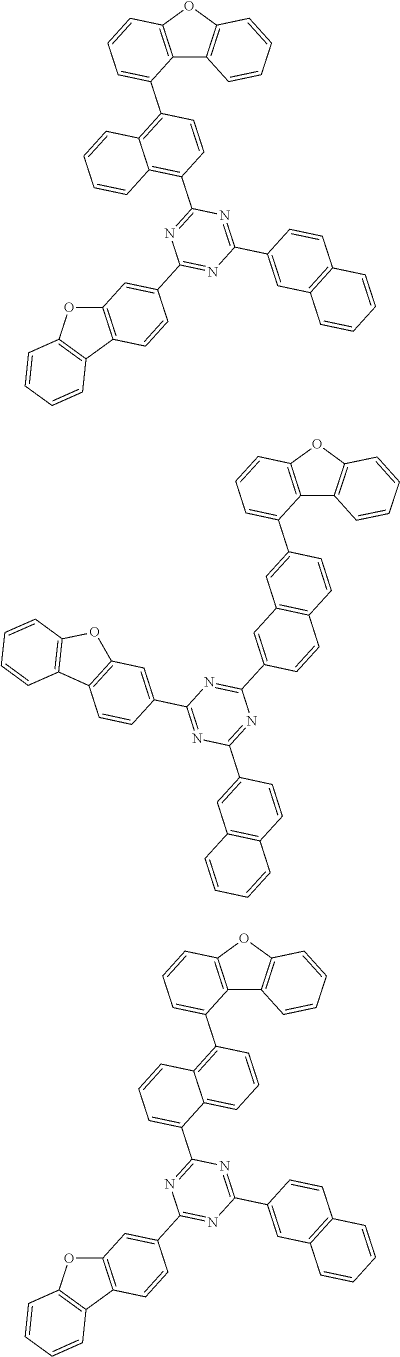

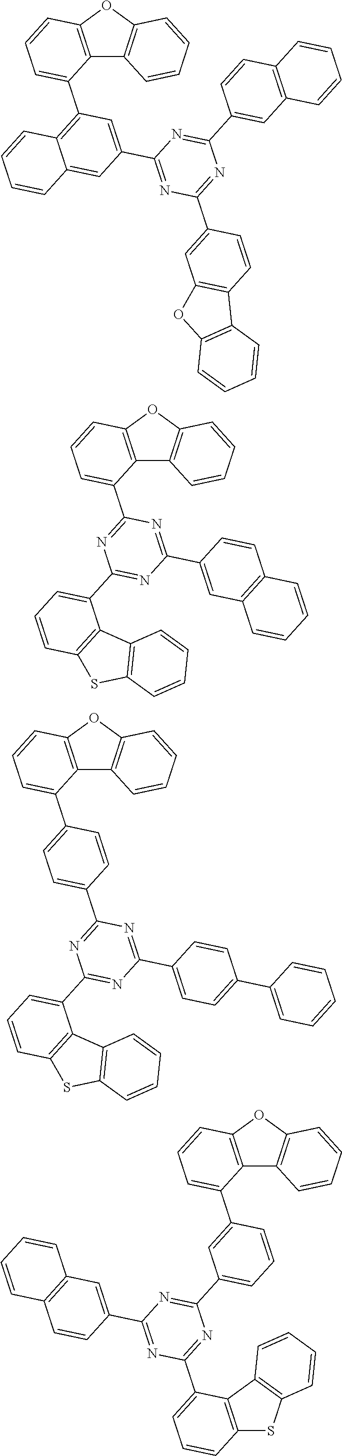

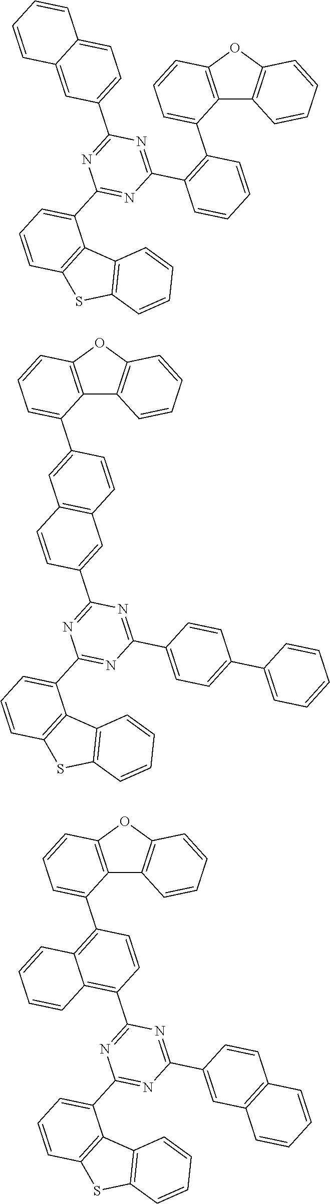

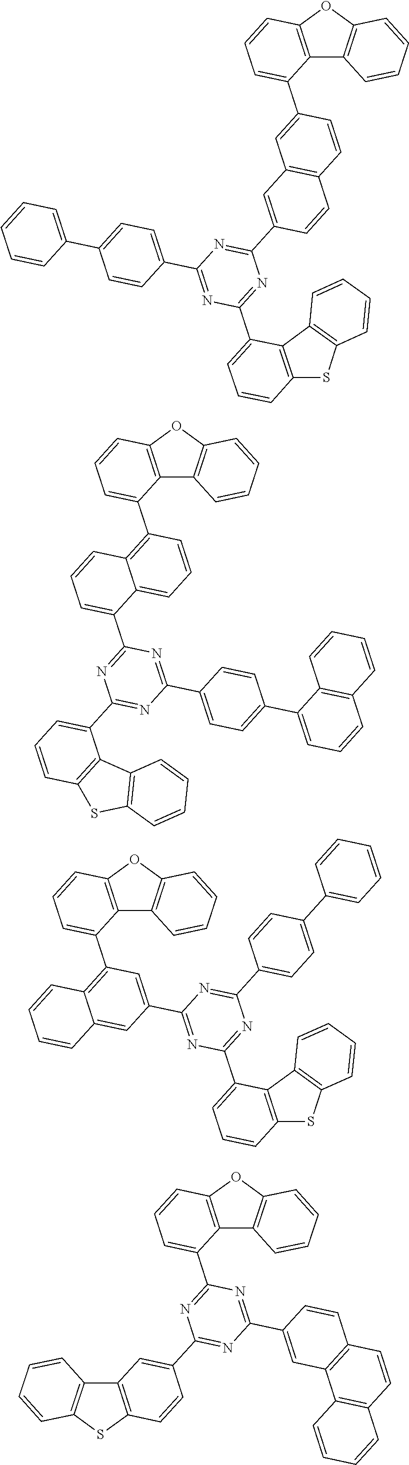

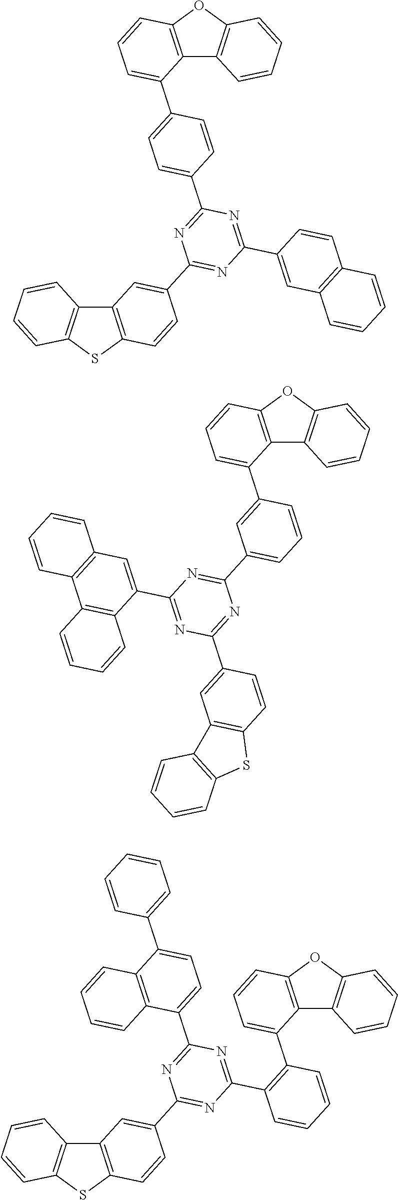

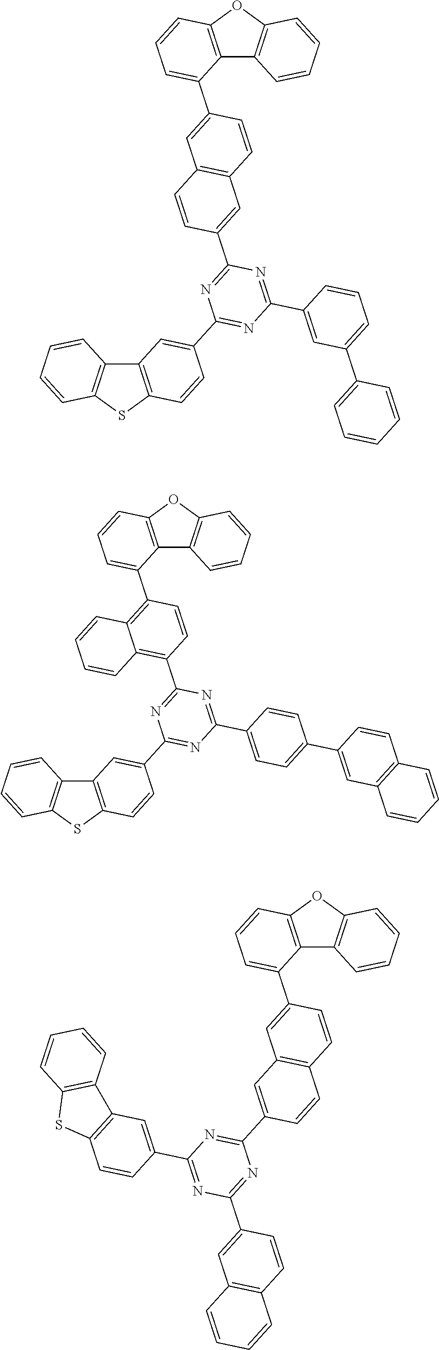 [SMILES: c1ccc(-c2cccc(-c3nc(-c4ccc5cc(-c6cccc7oc8ccccc8c67)ccc5c4)nc(-c4ccc5sc6ccccc6c5c4)n3)c2)cc1 ; c1ccc2cc(-c3ccc(-c4nc(-c5ccc6sc7ccccc7c6c5)nc(-c5ccc(-c6cccc7oc8ccccc8c67)c6ccccc56)n4)cc3)ccc2c1 ; c1ccc2cc(-c3nc(-c4ccc5ccc(-c6cccc7oc8ccccc8c67)cc5c4)nc(-c4ccc5sc6ccccc6c5c4)n3)ccc2c1]

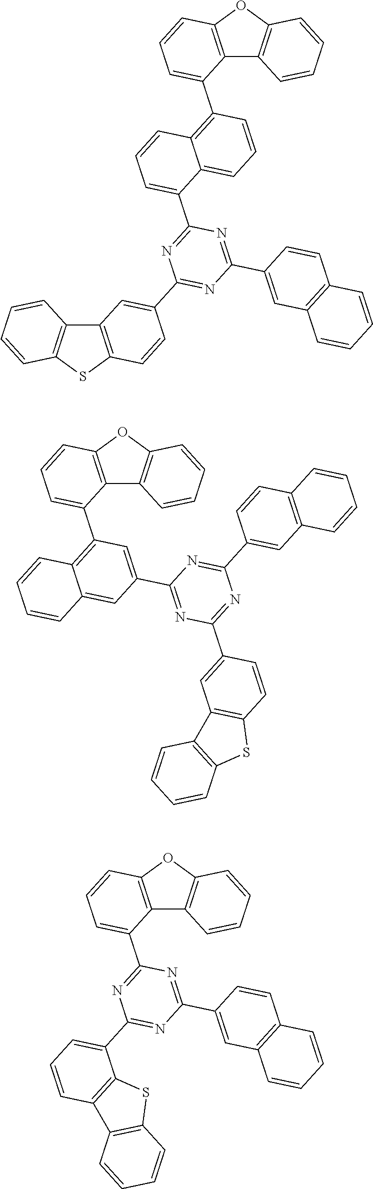 [SMILES: c1ccc2cc(-c3nc(-c4ccc5sc6ccccc6c5c4)nc(-c4cccc5c(-c6cccc7oc8ccccc8c67)cccc45)n3)ccc2c1 ; c1ccc2cc(-c3nc(-c4cc(-c5cccc6oc7ccccc7c56)c5ccccc5c4)nc(-c4ccc5sc6ccccc6c5c4)n3)ccc2c1 ; c1ccc2cc(-c3nc(-c4cccc5c4sc4ccccc45)nc(-c4cccc5oc6ccccc6c45)n3)ccc2c1]

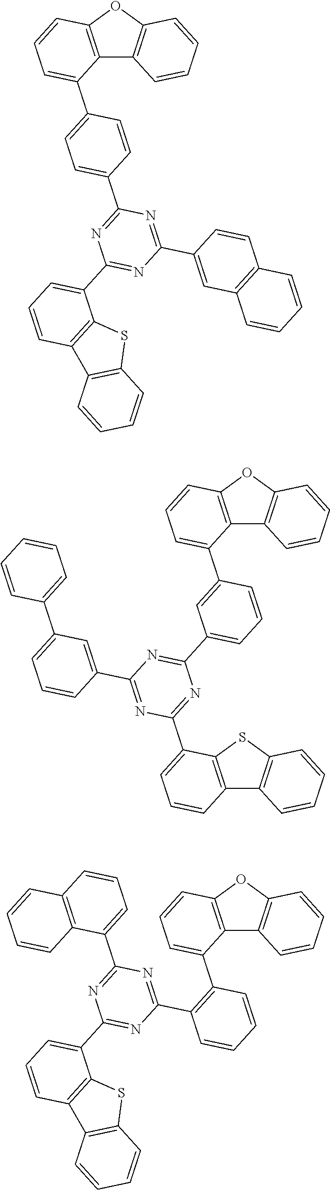 [SMILES: c1ccc2cc(-c3nc(-c4ccc(-c5cccc6oc7ccccc7c56)cc4)nc(-c4cccc5c4sc4ccccc45)n3)ccc2c1 ; c1ccc(-c2cccc(-c3nc(-c4cccc(-c5cccc6oc7ccccc7c56)c4)nc(-c4cccc5c4sc4ccccc45)n3)c2)cc1 ; c1ccc(-c2cccc3oc4ccccc4c23)c(-c2nc(-c3cccc4ccccc34)nc(-c3cccc4c3sc3ccccc34)n2)c1]

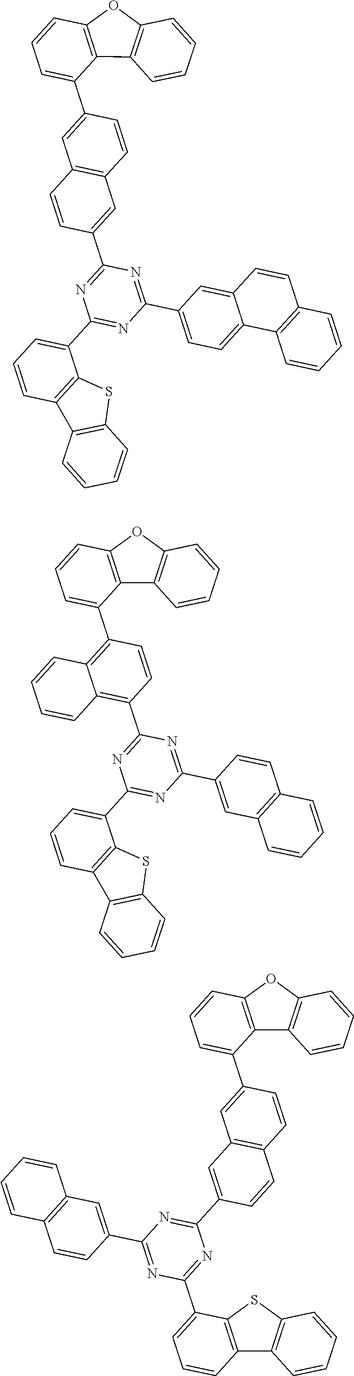 [SMILES: c1ccc2c(c1)ccc1cc(-c3nc(-c4ccc5cc(-c6cccc7oc8ccccc8c67)ccc5c4)nc(-c4cccc5c4sc4ccccc45)n3)ccc12 ; c1ccc2cc(-c3nc(-c4ccc(-c5cccc6oc7ccccc7c56)c5ccccc45)nc(-c4cccc5c4sc4ccccc45)n3)ccc2c1 ; c1ccc2cc(-c3nc(-c4ccc5ccc(-c6cccc7oc8ccccc8c67)cc5c4)nc(-c4cccc5c4sc4ccccc45)n3)ccc2c1]

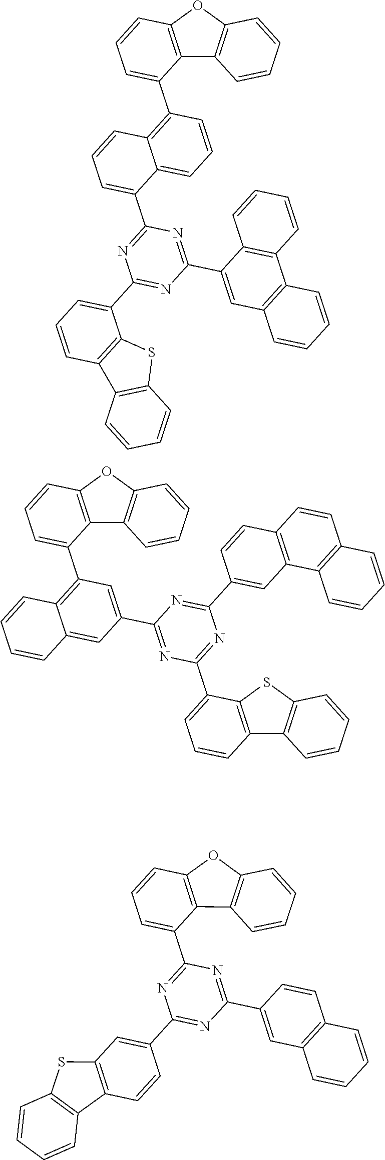

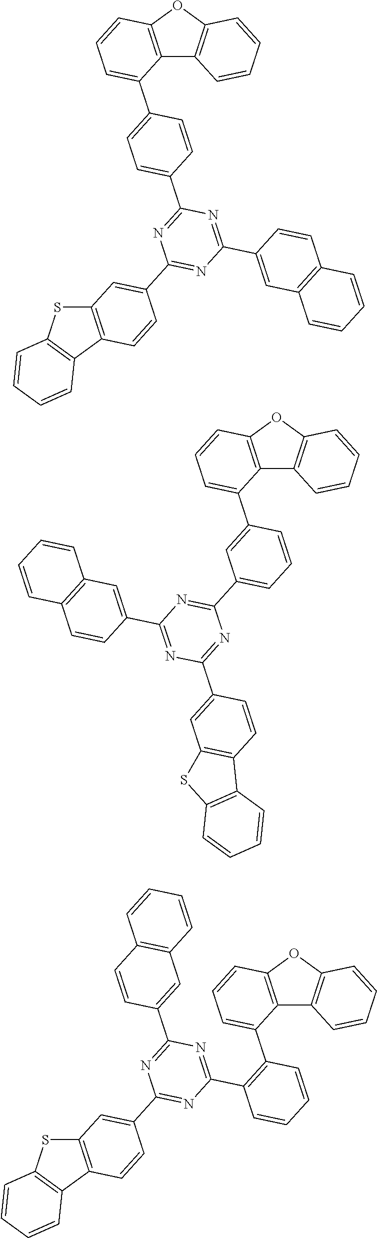 [SMILES: c1ccc2cc(-c3nc(-c4ccc(-c5cccc6oc7ccccc7c56)cc4)nc(-c4ccc5c(c4)sc4ccccc45)n3)ccc2c1 ; c1cc(-c2nc(-c3ccc4ccccc4c3)nc(-c3ccc4c(c3)sc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)sc3ccccc34)n2)c1]

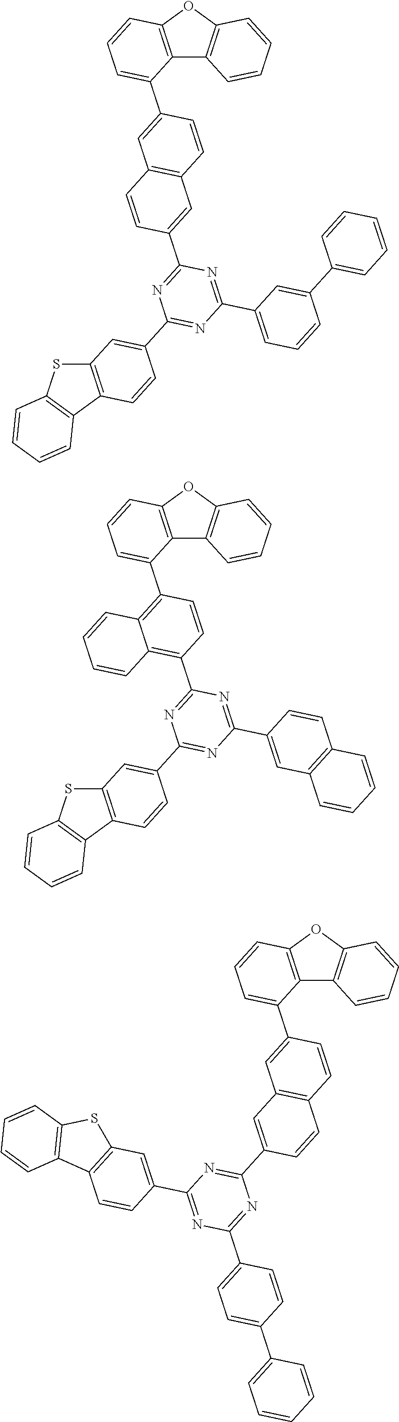 [SMILES: c1ccc(-c2cccc(-c3nc(-c4ccc5cc(-c6cccc7oc8ccccc8c67)ccc5c4)nc(-c4ccc5c(c4)sc4ccccc45)n3)c2)cc1 ; c1ccc2cc(-c3nc(-c4ccc5c(c4)sc4ccccc45)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(c4)sc4ccccc45)n3)cc2)cc1]

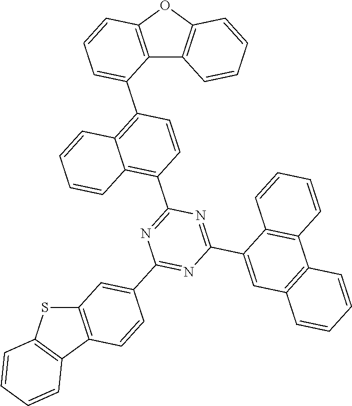 [SMILES: c1ccc2c(c1)cc(-c1nc(-c3ccc4c(c3)sc3ccccc34)nc(-c3ccc(-c4cccc5oc6ccccc6c45)c4ccccc34)n1)c1ccccc12]

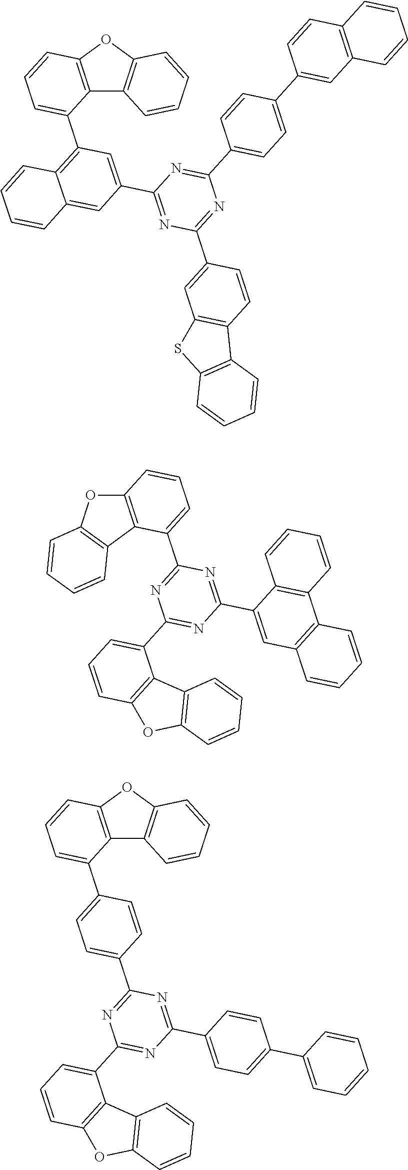 [SMILES: c1ccc2cc(-c3ccc(-c4nc(-c5cc(-c6cccc7oc8ccccc8c67)c6ccccc6c5)nc(-c5ccc6c(c5)sc5ccccc56)n4)cc3)ccc2c1 ; c1ccc2c(c1)cc(-c1nc(-c3cccc4oc5ccccc5c34)nc(-c3cccc4oc5ccccc5c34)n1)c1ccccc12 ; c1ccc(-c2ccc(-c3nc(-c4ccc(-c5cccc6oc7ccccc7c56)cc4)nc(-c4cccc5oc6ccccc6c45)n3)cc2)cc1]

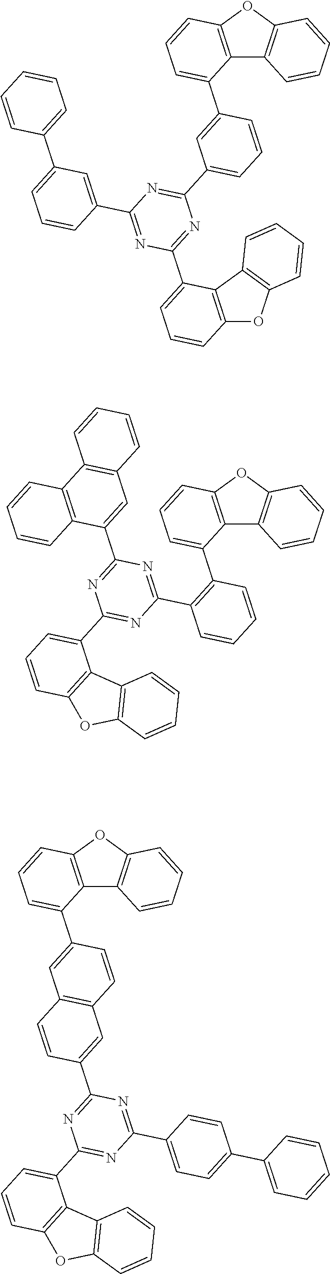 [SMILES: c1ccc(-c2cccc(-c3nc(-c4cccc(-c5cccc6oc7ccccc7c56)c4)nc(-c4cccc5oc6ccccc6c45)n3)c2)cc1 ; c1ccc(-c2cccc3oc4ccccc4c23)c(-c2nc(-c3cc4ccccc4c4ccccc34)nc(-c3cccc4oc5ccccc5c34)n2)c1 ; c1ccc(-c2ccc(-c3nc(-c4ccc5cc(-c6cccc7oc8ccccc8c67)ccc5c4)nc(-c4cccc5oc6ccccc6c45)n3)cc2)cc1]

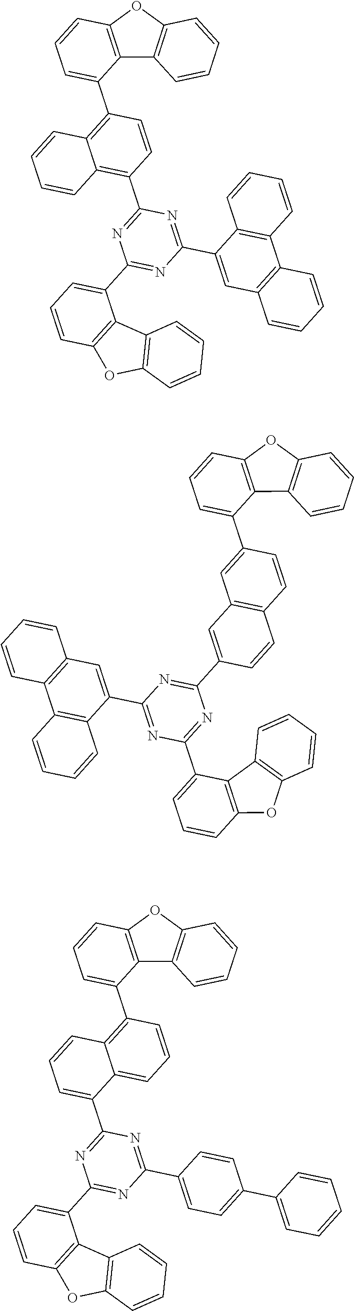 [SMILES: c1ccc2c(c1)cc(-c1nc(-c3ccc(-c4cccc5oc6ccccc6c45)c4ccccc34)nc(-c3cccc4oc5ccccc5c34)n1)c1ccccc12 ; c1ccc2c(c1)cc(-c1nc(-c3ccc4ccc(-c5cccc6oc7ccccc7c56)cc4c3)nc(-c3cccc4oc5ccccc5c34)n1)c1ccccc12 ; c1ccc(-c2ccc(-c3nc(-c4cccc5c(-c6cccc7oc8ccccc8c67)cccc45)nc(-c4cccc5oc6ccccc6c45)n3)cc2)cc1]

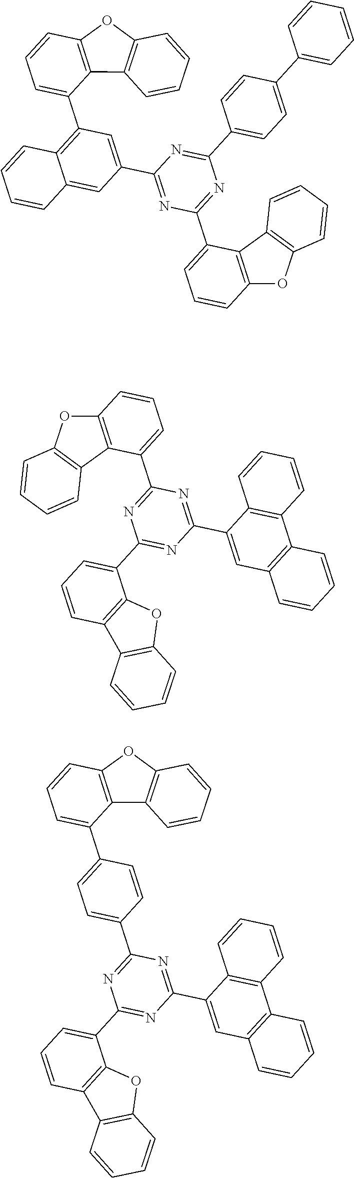

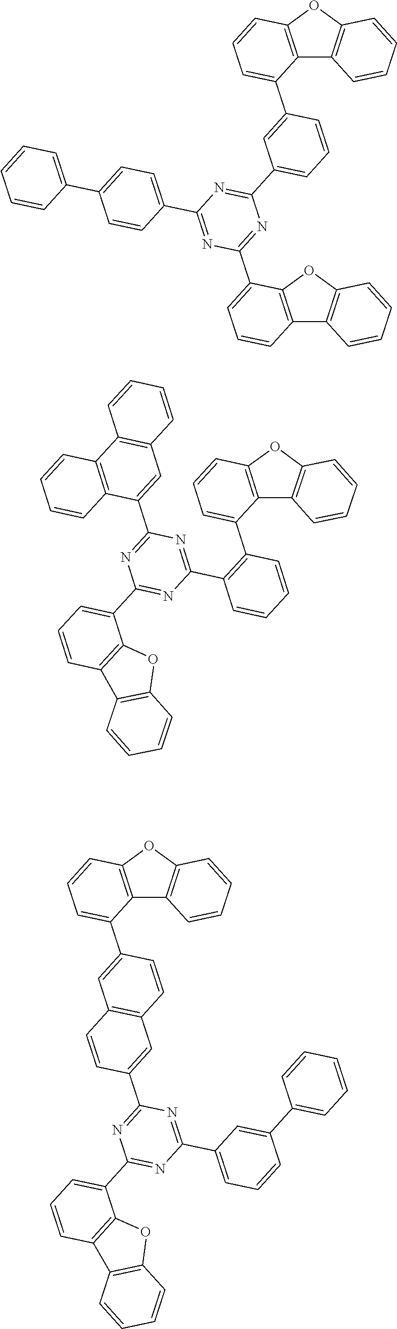 [SMILES: c1ccc(-c2ccc(-c3nc(-c4cccc(-c5cccc6oc7ccccc7c56)c4)nc(-c4cccc5c4oc4ccccc45)n3)cc2)cc1 ; c1ccc(-c2cccc3oc4ccccc4c23)c(-c2nc(-c3cc4ccccc4c4ccccc34)nc(-c3cccc4c3oc3ccccc34)n2)c1 ; c1ccc(-c2cccc(-c3nc(-c4ccc5cc(-c6cccc7oc8ccccc8c67)ccc5c4)nc(-c4cccc5c4oc4ccccc45)n3)c2)cc1]

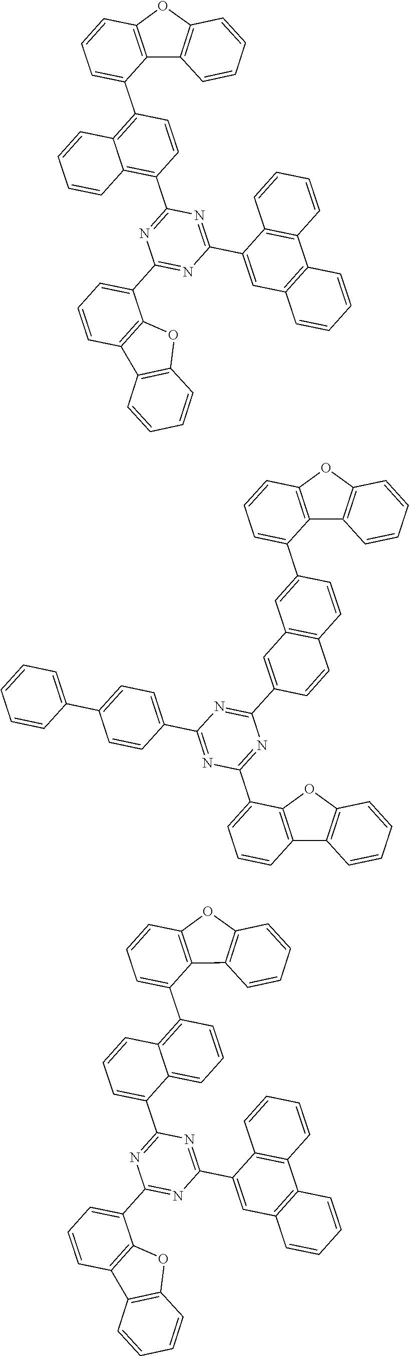 [SMILES: c1ccc2c(c1)cc(-c1nc(-c3ccc(-c4cccc5oc6ccccc6c45)c4ccccc34)nc(-c3cccc4c3oc3ccccc34)n1)c1ccccc12 ; c1ccc(-c2ccc(-c3nc(-c4ccc5ccc(-c6cccc7oc8ccccc8c67)cc5c4)nc(-c4cccc5c4oc4ccccc45)n3)cc2)cc1 ; c1ccc2c(c1)cc(-c1nc(-c3cccc4c(-c5cccc6oc7ccccc7c56)cccc34)nc(-c3cccc4c3oc3ccccc34)n1)c1ccccc12]

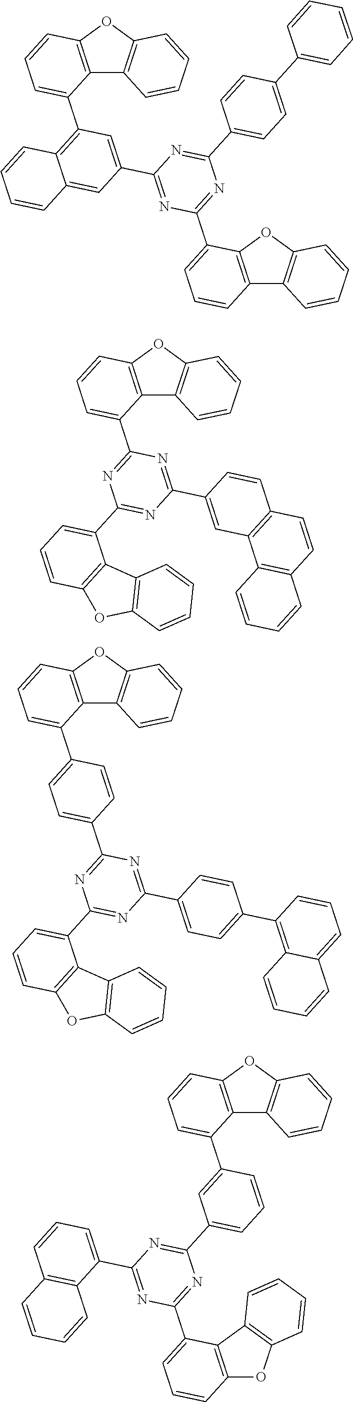

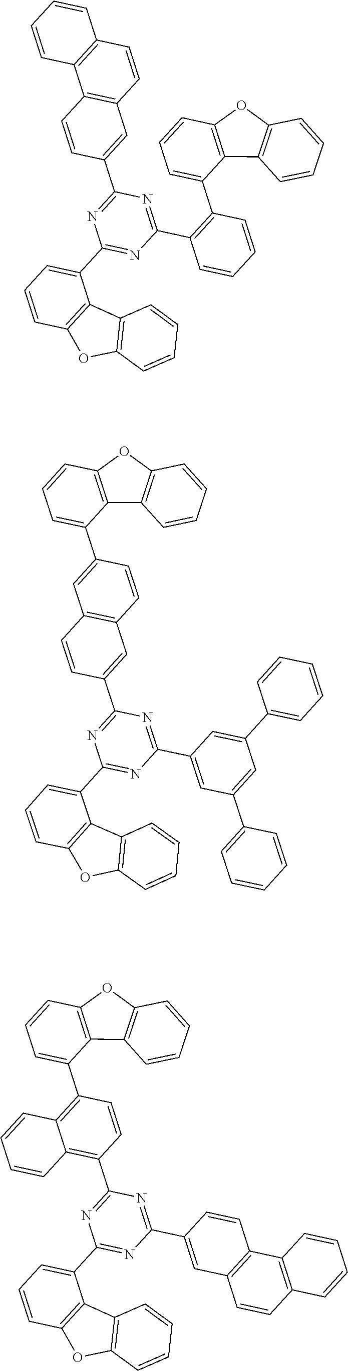 [SMILES: c1ccc(-c2cccc3oc4ccccc4c23)c(-c2nc(-c3ccc4c(ccc5ccccc54)c3)nc(-c3cccc4oc5ccccc5c34)n2)c1 ; c1ccc(-c2cc(-c3ccccc3)cc(-c3nc(-c4ccc5cc(-c6cccc7oc8ccccc8c67)ccc5c4)nc(-c4cccc5oc6ccccc6c45)n3)c2)cc1 ; c1ccc2c(c1)ccc1cc(-c3nc(-c4ccc(-c5cccc6oc7ccccc7c56)c5ccccc45)nc(-c4cccc5oc6ccccc6c45)n3)ccc12]

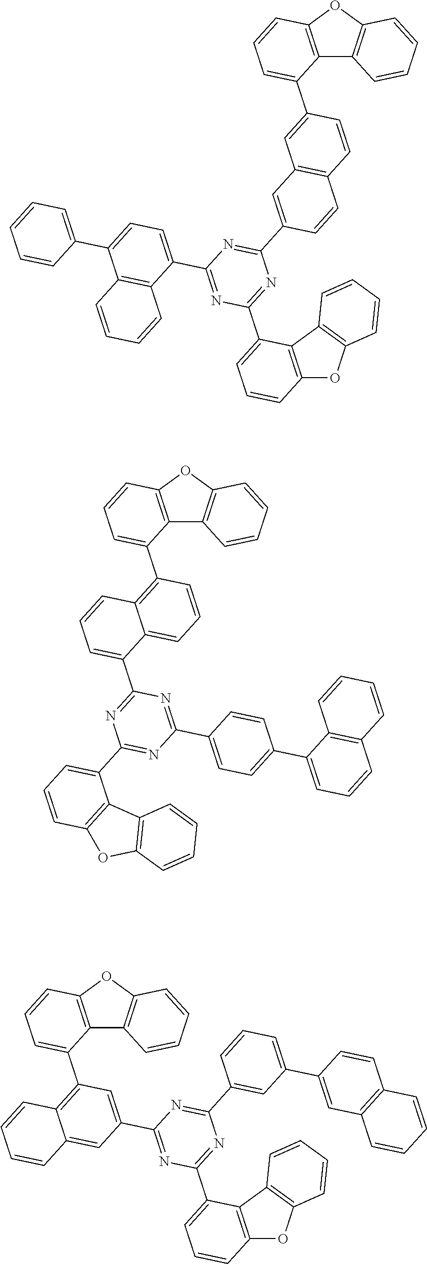 [SMILES: c1ccc(-c2ccc(-c3nc(-c4ccc5ccc(-c6cccc7oc8ccccc8c67)cc5c4)nc(-c4cccc5oc6ccccc6c45)n3)c3ccccc23)cc1 ; c1ccc2c(-c3ccc(-c4nc(-c5cccc6c(-c7cccc8oc9ccccc9c78)cccc56)nc(-c5cccc6oc7ccccc7c56)n4)cc3)cccc2c1 ; c1cc(-c2ccc3ccccc3c2)cc(-c2nc(-c3cc(-c4cccc5oc6ccccc6c45)c4ccccc4c3)nc(-c3cccc4oc5ccccc5c34)n2)c1]

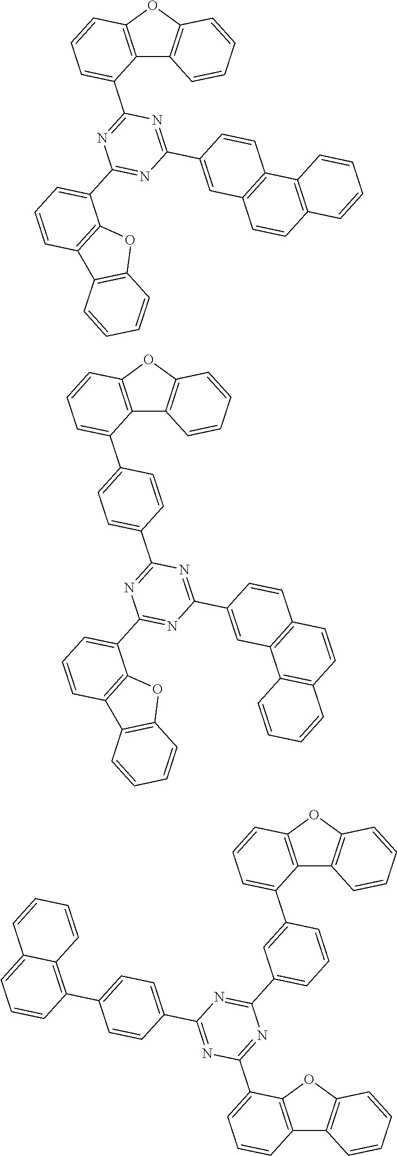

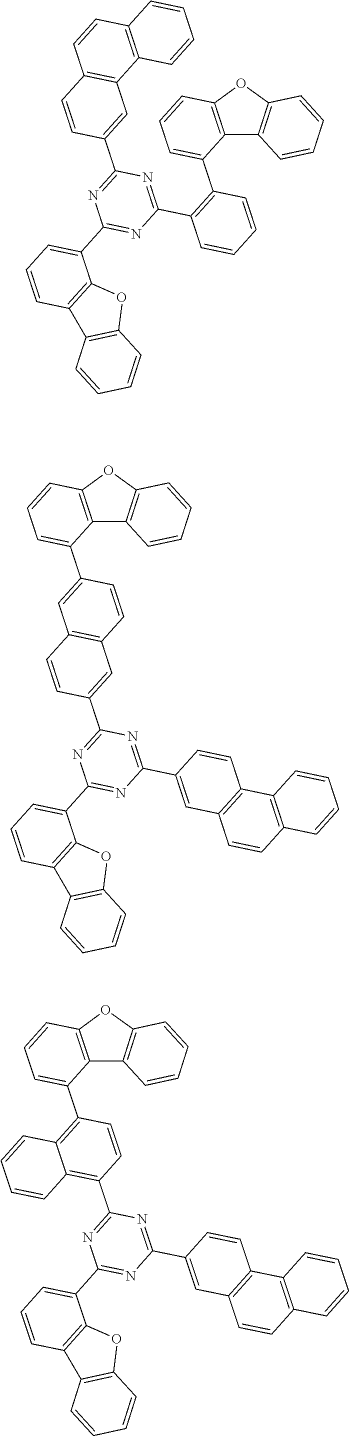 [SMILES: c1ccc(-c2cccc3oc4ccccc4c23)c(-c2nc(-c3ccc4ccc5ccccc5c4c3)nc(-c3cccc4c3oc3ccccc34)n2)c1 ; c1ccc2c(c1)ccc1cc(-c3nc(-c4ccc5cc(-c6cccc7oc8ccccc8c67)ccc5c4)nc(-c4cccc5c4oc4ccccc45)n3)ccc12 ; c1ccc2c(c1)ccc1cc(-c3nc(-c4ccc(-c5cccc6oc7ccccc7c56)c5ccccc45)nc(-c4cccc5c4oc4ccccc45)n3)ccc12]

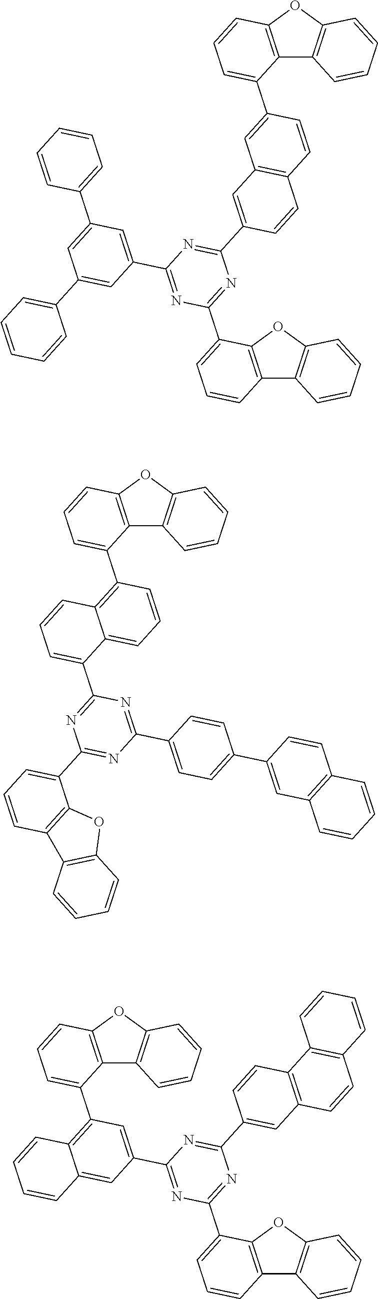 [SMILES: c1ccc(-c2cc(-c3ccccc3)cc(-c3nc(-c4ccc5ccc(-c6cccc7oc8ccccc8c67)cc5c4)nc(-c4cccc5c4oc4ccccc45)n3)c2)cc1 ; c1ccc2cc(-c3ccc(-c4nc(-c5cccc6c(-c7cccc8oc9ccccc9c78)cccc56)nc(-c5cccc6c5oc5ccccc56)n4)cc3)ccc2c1 ; c1ccc2c(-c3cccc4oc5ccccc5c34)cc(-c3nc(-c4ccc5c(ccc6ccccc65)c4)nc(-c4cccc5c4oc4ccccc45)n3)cc2c1]

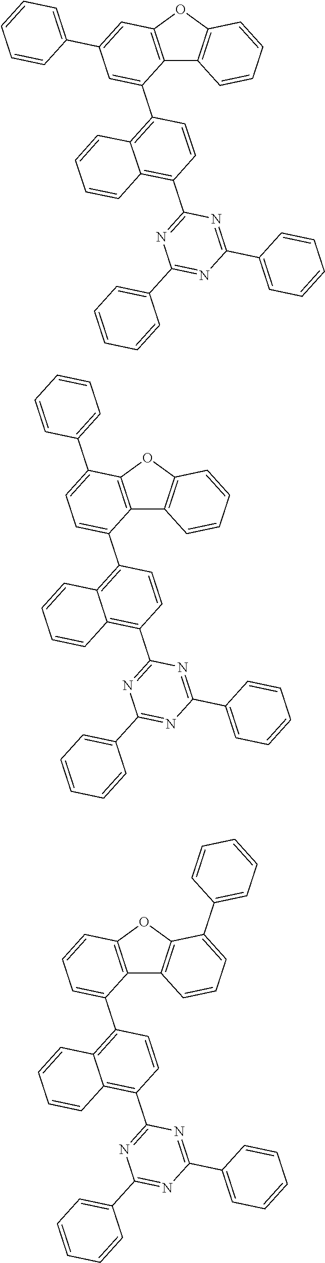

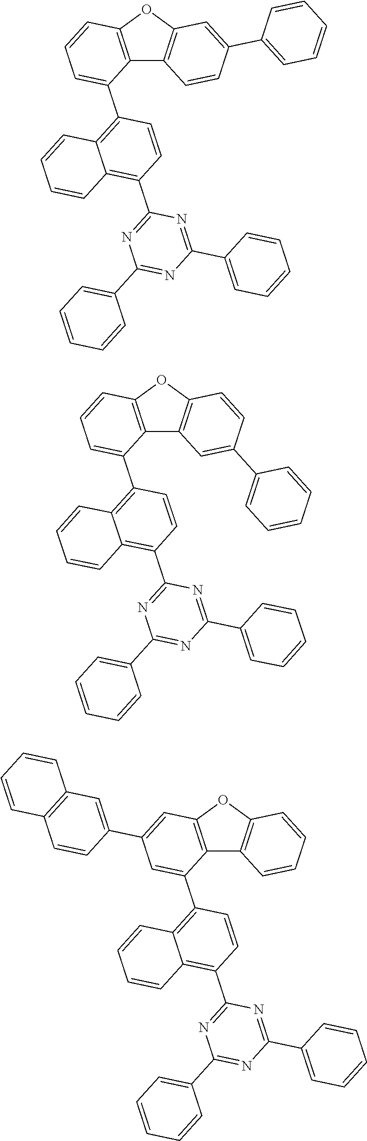

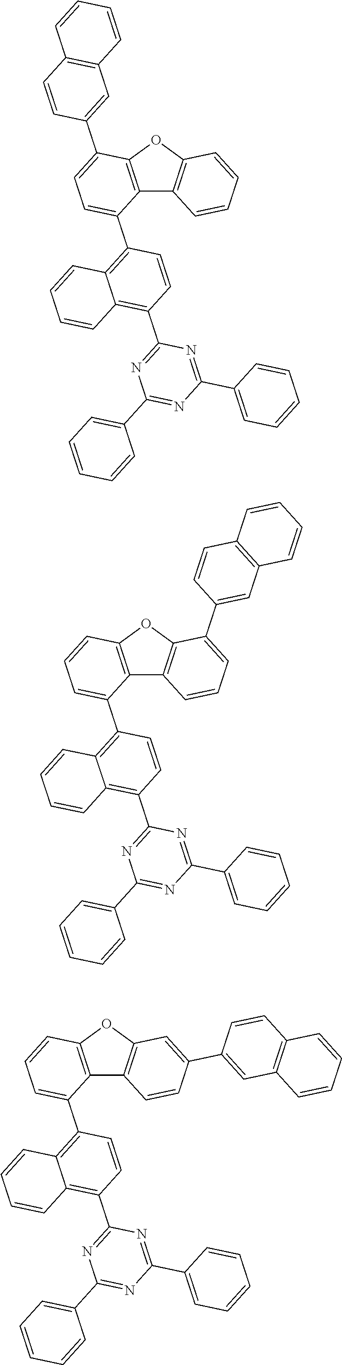 [SMILES: c1ccc(-c2nc(-c3ccccc3)nc(-c3ccc(-c4ccc(-c5ccc6ccccc6c5)c5oc6ccccc6c45)c4ccccc34)n2)cc1 ; c1ccc(-c2nc(-c3ccccc3)nc(-c3ccc(-c4cccc5oc6c(-c7ccc8ccccc8c7)cccc6c45)c4ccccc34)n2)cc1 ; c1ccc(-c2nc(-c3ccccc3)nc(-c3ccc(-c4cccc5oc6cc(-c7ccc8ccccc8c7)ccc6c45)c4ccccc34)n2)cc1]

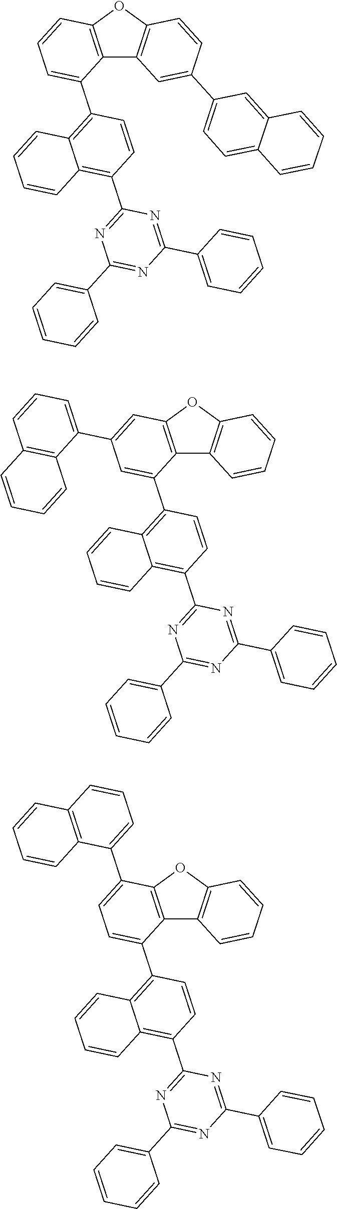 [SMILES: c1ccc(-c2nc(-c3ccccc3)nc(-c3ccc(-c4cccc5oc6ccc(-c7ccc8ccccc8c7)cc6c45)c4ccccc34)n2)cc1 ; c1ccc(-c2nc(-c3ccccc3)nc(-c3ccc(-c4cc(-c5cccc6ccccc56)cc5oc6ccccc6c45)c4ccccc34)n2)cc1 ; c1ccc(-c2nc(-c3ccccc3)nc(-c3ccc(-c4ccc(-c5cccc6ccccc56)c5oc6ccccc6c45)c4ccccc34)n2)cc1]

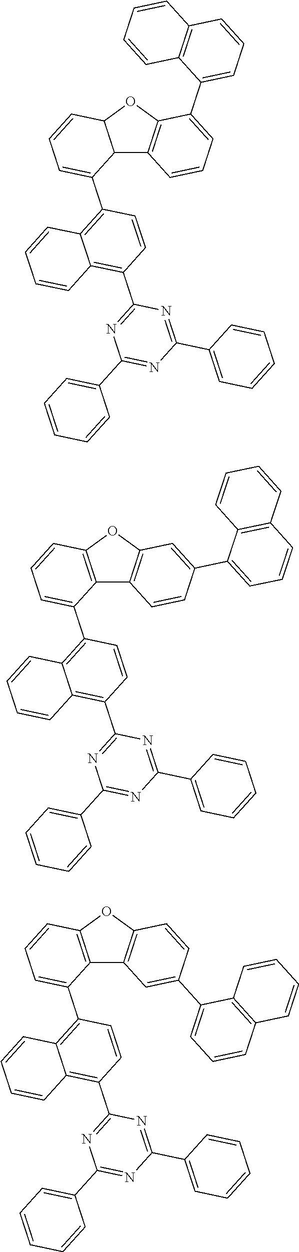 [SMILES: C1=CC2Oc3c(-c4cccc5ccccc45)cccc3C2C(c2ccc(-c3nc(-c4ccccc4)nc(-c4ccccc4)n3)c3ccccc23)=C1 ; c1ccc(-c2nc(-c3ccccc3)nc(-c3ccc(-c4cccc5oc6cc(-c7cccc8ccccc78)ccc6c45)c4ccccc34)n2)cc1 ; c1ccc(-c2nc(-c3ccccc3)nc(-c3ccc(-c4cccc5oc6ccc(-c7cccc8ccccc78)cc6c45)c4ccccc34)n2)cc1]

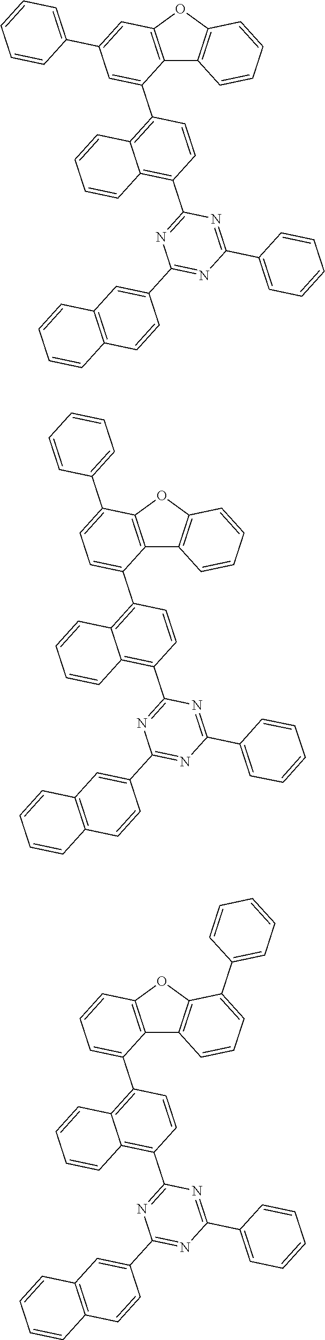

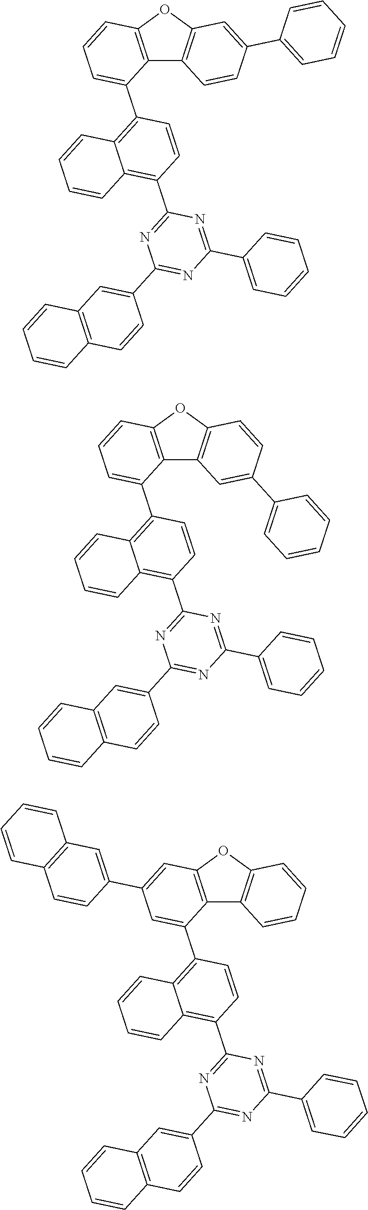 [SMILES: c1ccc(-c2ccc3c(c2)oc2cccc(-c4ccc(-c5nc(-c6ccccc6)nc(-c6ccc7ccccc7c6)n5)c5ccccc45)c23)cc1 ; c1ccc(-c2ccc3oc4cccc(-c5ccc(-c6nc(-c7ccccc7)nc(-c7ccc8ccccc8c7)n6)c6ccccc56)c4c3c2)cc1 ; c1ccc(-c2nc(-c3ccc4ccccc4c3)nc(-c3ccc(-c4cc(-c5ccc6ccccc6c5)cc5oc6ccccc6c45)c4ccccc34)n2)cc1]

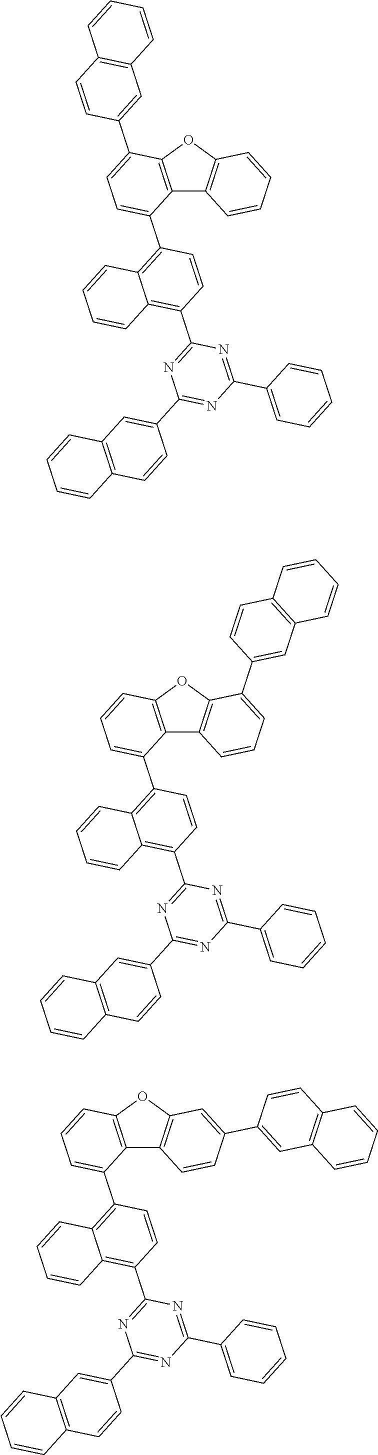 [SMILES: c1ccc(-c2nc(-c3ccc4ccccc4c3)nc(-c3ccc(-c4ccc(-c5ccc6ccccc6c5)c5oc6ccccc6c45)c4ccccc34)n2)cc1 ; c1ccc(-c2nc(-c3ccc4ccccc4c3)nc(-c3ccc(-c4cccc5oc6c(-c7ccc8ccccc8c7)cccc6c45)c4ccccc34)n2)cc1 ; c1ccc(-c2nc(-c3ccc4ccccc4c3)nc(-c3ccc(-c4cccc5oc6cc(-c7ccc8ccccc8c7)ccc6c45)c4ccccc34)n2)cc1]

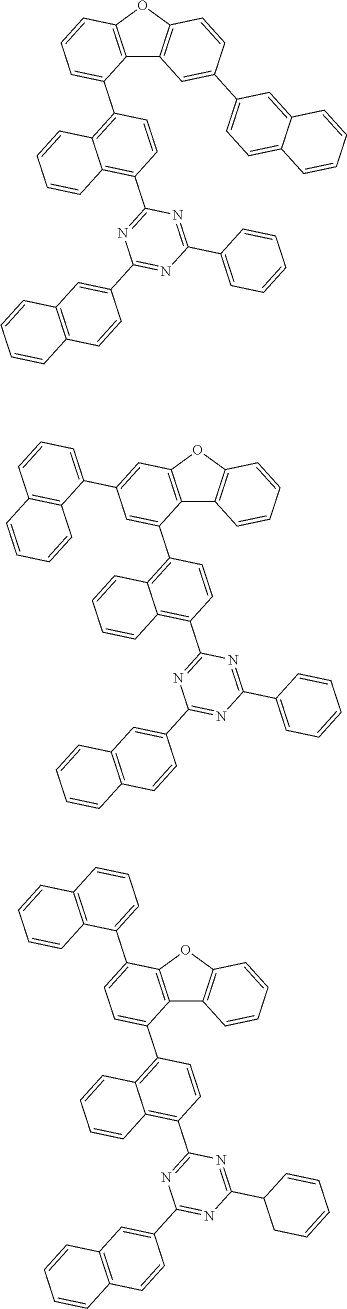 [SMILES: c1ccc(-c2nc(-c3ccc4ccccc4c3)nc(-c3ccc(-c4cccc5oc6ccc(-c7ccc8ccccc8c7)cc6c45)c4ccccc34)n2)cc1 ; c1ccc(-c2nc(-c3ccc4ccccc4c3)nc(-c3ccc(-c4cc(-c5cccc6ccccc56)cc5oc6ccccc6c45)c4ccccc34)n2)cc1 ; C1=CCC(c2nc(-c3ccc4ccccc4c3)nc(-c3ccc(-c4ccc(-c5cccc6ccccc56)c5oc6ccccc6c45)c4ccccc34)n2)C=C1]

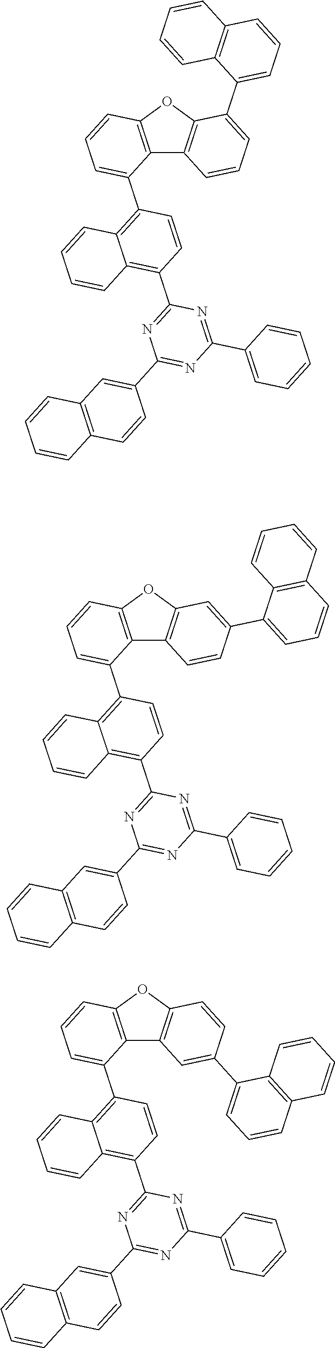 [SMILES: c1ccc(-c2nc(-c3ccc4ccccc4c3)nc(-c3ccc(-c4cccc5oc6c(-c7cccc8ccccc78)cccc6c45)c4ccccc34)n2)cc1 ; c1ccc(-c2nc(-c3ccc4ccccc4c3)nc(-c3ccc(-c4cccc5oc6cc(-c7cccc8ccccc78)ccc6c45)c4ccccc34)n2)cc1 ; c1ccc(-c2nc(-c3ccc4ccccc4c3)nc(-c3ccc(-c4cccc5oc6ccc(-c7cccc8ccccc78)cc6c45)c4ccccc34)n2)cc1]

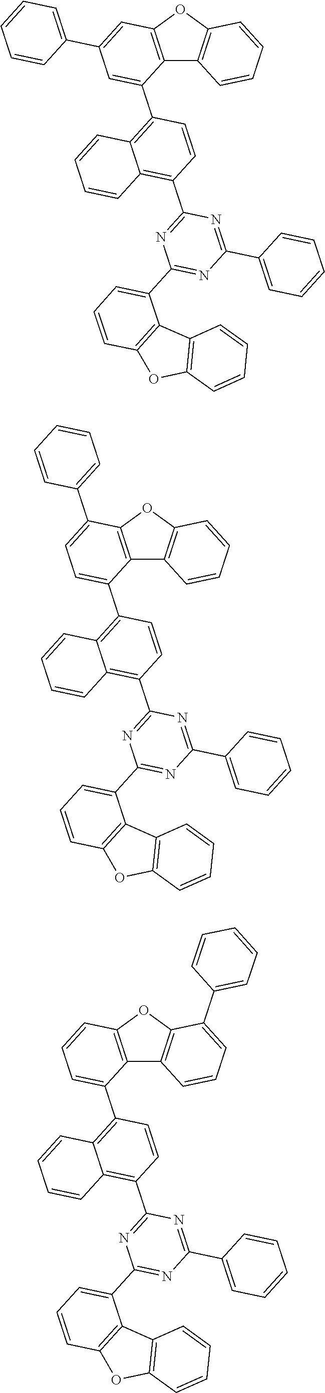 [SMILES: c1ccc(-c2cc(-c3ccc(-c4nc(-c5ccccc5)nc(-c5cccc6oc7ccccc7c56)n4)c4ccccc34)c3c(c2)oc2ccccc23)cc1 ; c1ccc(-c2nc(-c3ccc(-c4ccc(-c5ccccc5)c5oc6ccccc6c45)c4ccccc34)nc(-c3cccc4oc5ccccc5c34)n2)cc1 ; c1ccc(-c2nc(-c3ccc(-c4cccc5oc6c(-c7ccccc7)cccc6c45)c4ccccc34)nc(-c3cccc4oc5ccccc5c34)n2)cc1]

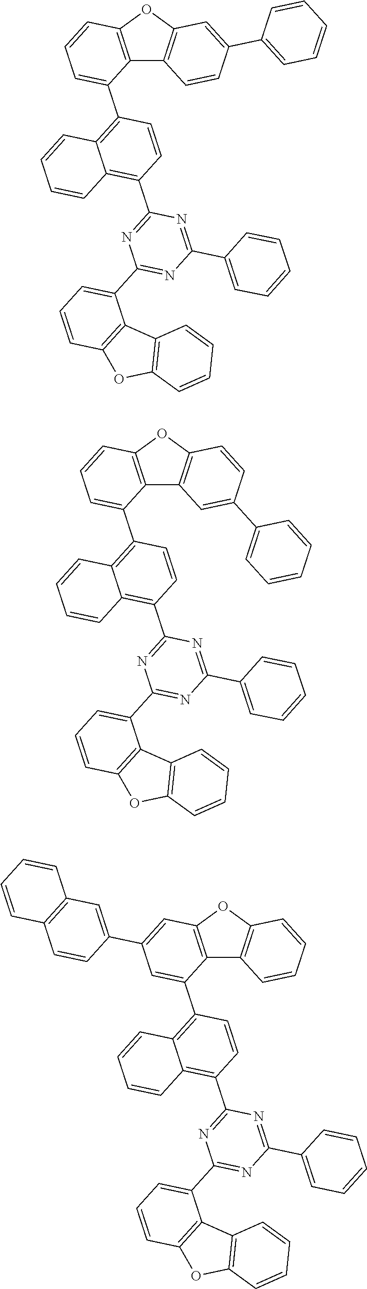

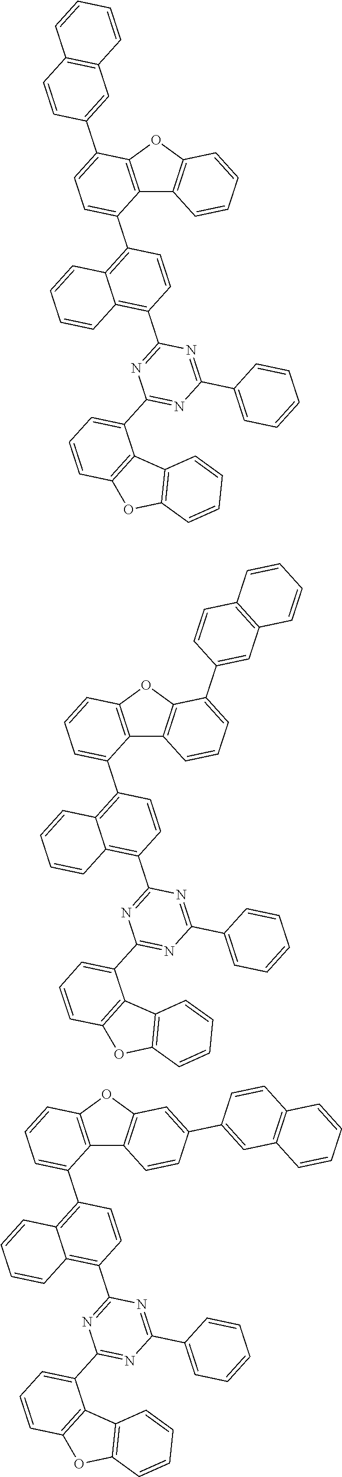

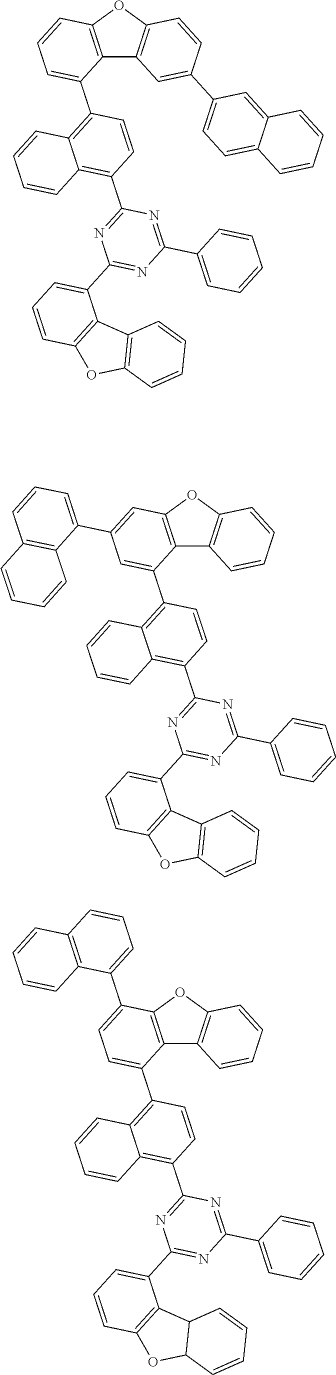 [SMILES: c1ccc(-c2nc(-c3ccc(-c4cccc5oc6ccc(-c7ccc8ccccc8c7)cc6c45)c4ccccc34)nc(-c3cccc4oc5ccccc5c34)n2)cc1 ; c1ccc(-c2nc(-c3ccc(-c4cc(-c5cccc6ccccc56)cc5oc6ccccc6c45)c4ccccc34)nc(-c3cccc4oc5ccccc5c34)n2)cc1 ; C1=CC2Oc3cccc(-c4nc(-c5ccccc5)nc(-c5ccc(-c6ccc(-c7cccc8ccccc78)c7oc8ccccc8c67)c6ccccc56)n4)c3C2C=C1]

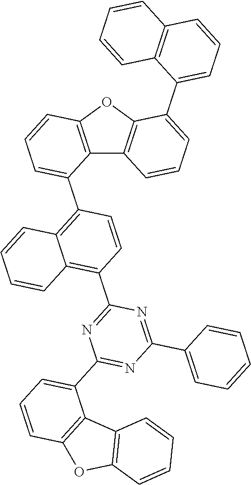 [SMILES: c1ccc(-c2nc(-c3ccc(-c4cccc5oc6c(-c7cccc8ccccc78)cccc6c45)c4ccccc34)nc(-c3cccc4oc5ccccc5c34)n2)cc1]

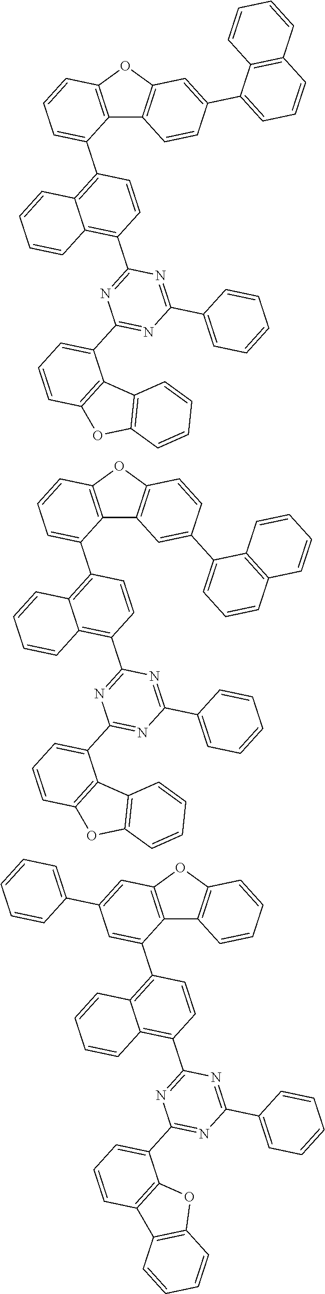

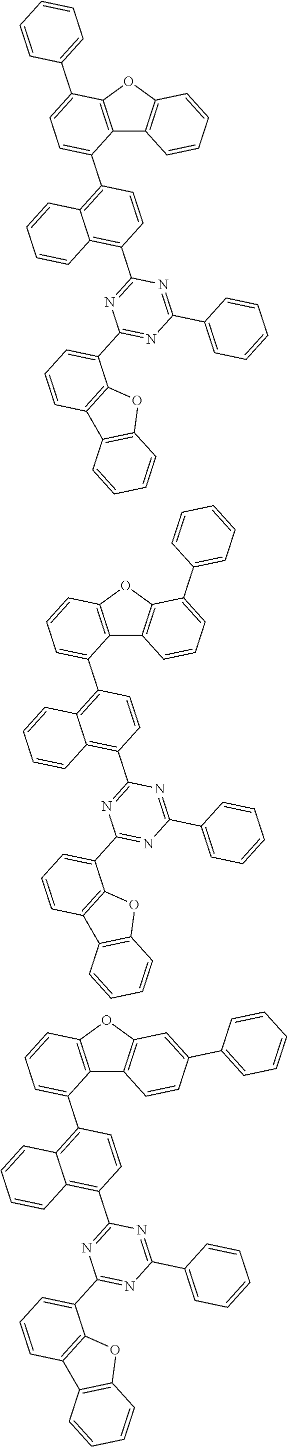

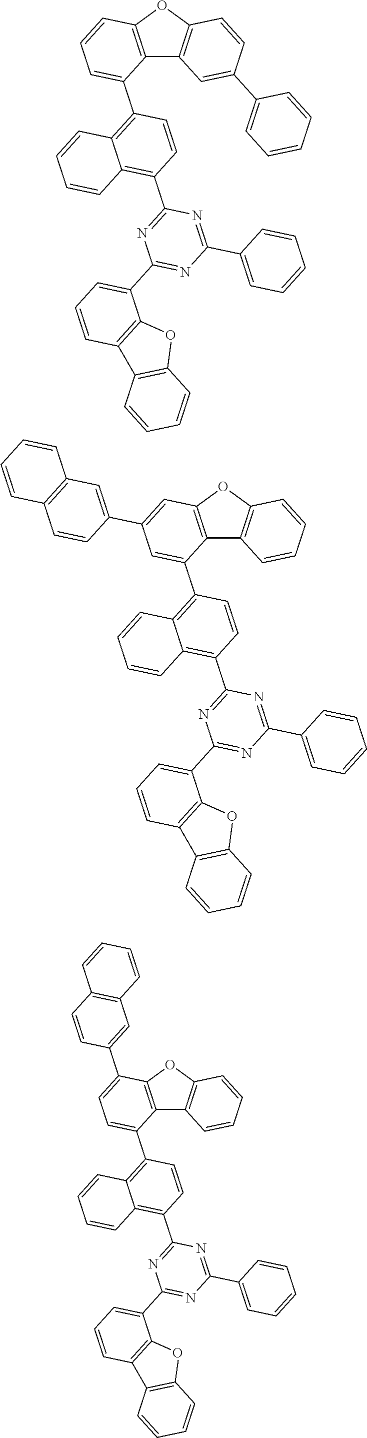 [SMILES: c1ccc(-c2ccc3oc4cccc(-c5ccc(-c6nc(-c7ccccc7)nc(-c7cccc8c7oc7ccccc78)n6)c6ccccc56)c4c3c2)cc1 ; c1ccc(-c2nc(-c3ccc(-c4cc(-c5ccc6ccccc6c5)cc5oc6ccccc6c45)c4ccccc34)nc(-c3cccc4c3oc3ccccc34)n2)cc1 ; c1ccc(-c2nc(-c3ccc(-c4ccc(-c5ccc6ccccc6c5)c5oc6ccccc6c45)c4ccccc34)nc(-c3cccc4c3oc3ccccc34)n2)cc1]

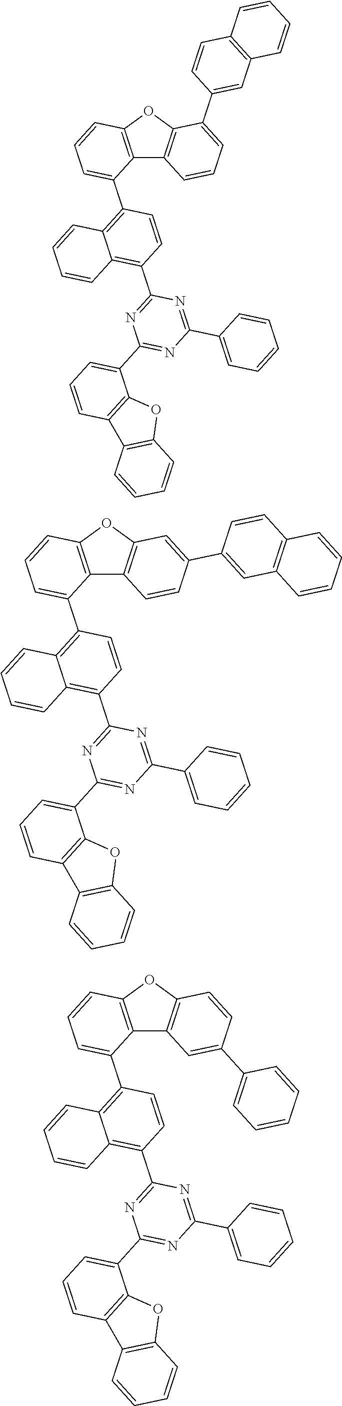

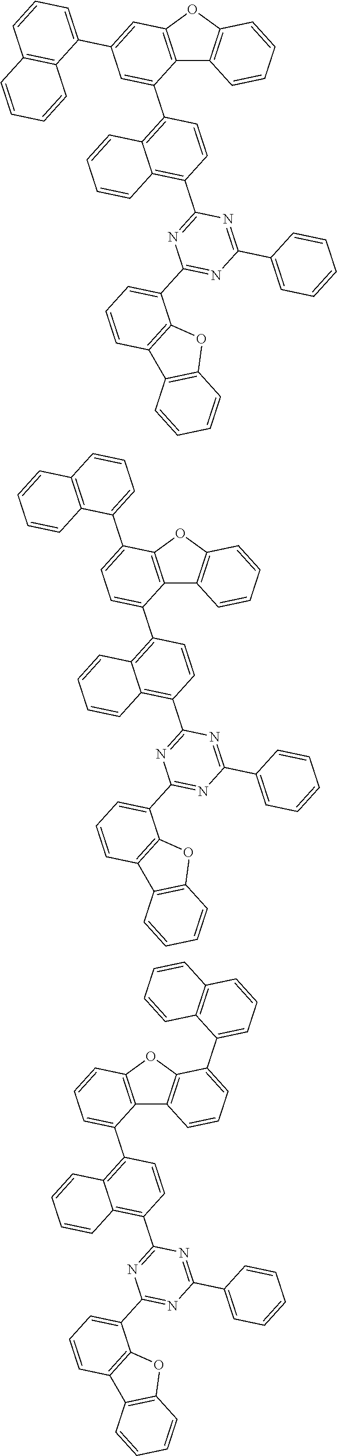

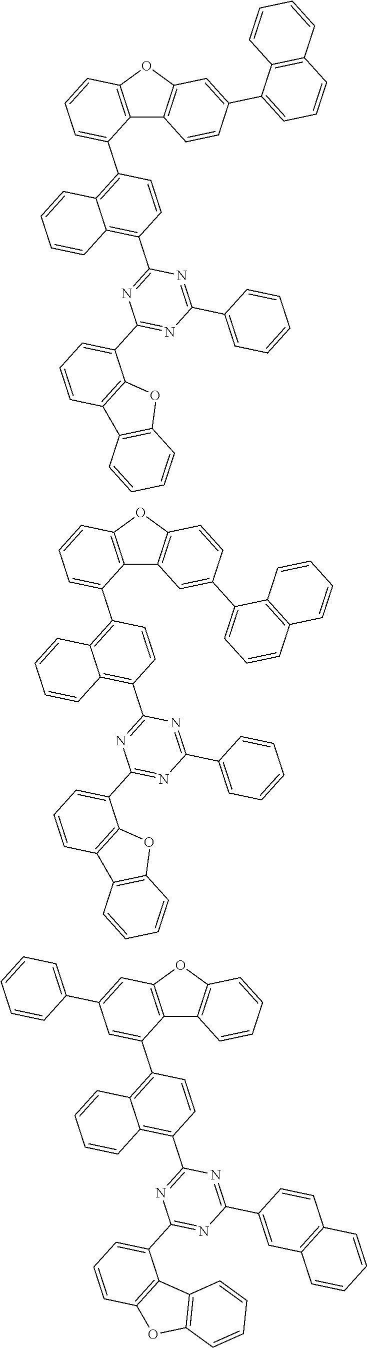

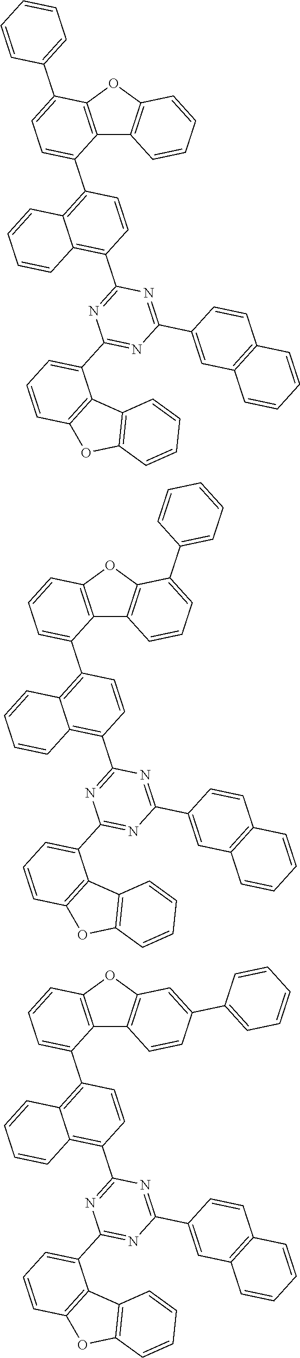

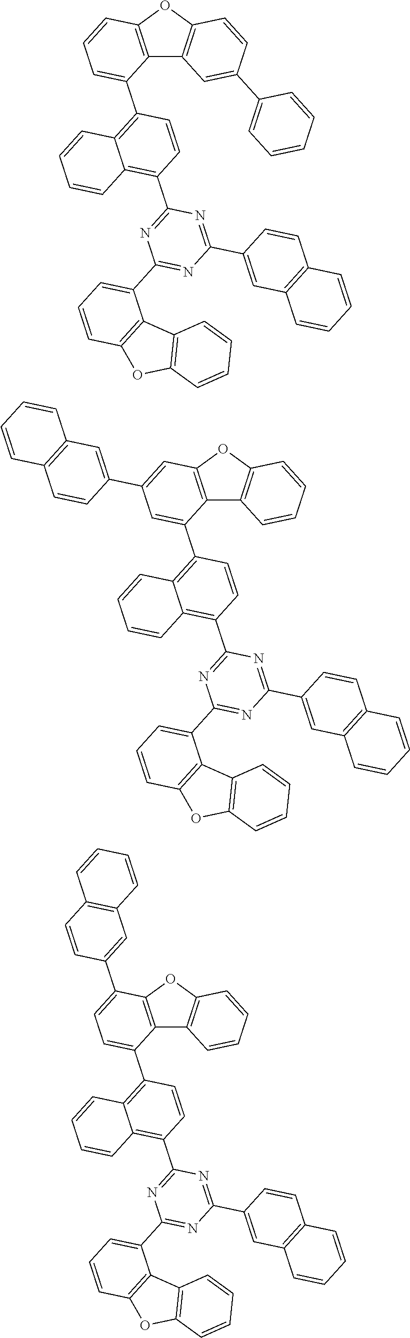 [SMILES: c1ccc(-c2ccc3oc4cccc(-c5ccc(-c6nc(-c7ccc8ccccc8c7)nc(-c7cccc8oc9ccccc9c78)n6)c6ccccc56)c4c3c2)cc1 ; c1ccc2cc(-c3cc(-c4ccc(-c5nc(-c6ccc7ccccc7c6)nc(-c6cccc7oc8ccccc8c67)n5)c5ccccc45)c4c(c3)oc3ccccc34)ccc2c1 ; c1ccc2cc(-c3nc(-c4ccc(-c5ccc(-c6ccc7ccccc7c6)c6oc7ccccc7c56)c5ccccc45)nc(-c4cccc5oc6ccccc6c45)n3)ccc2c1]

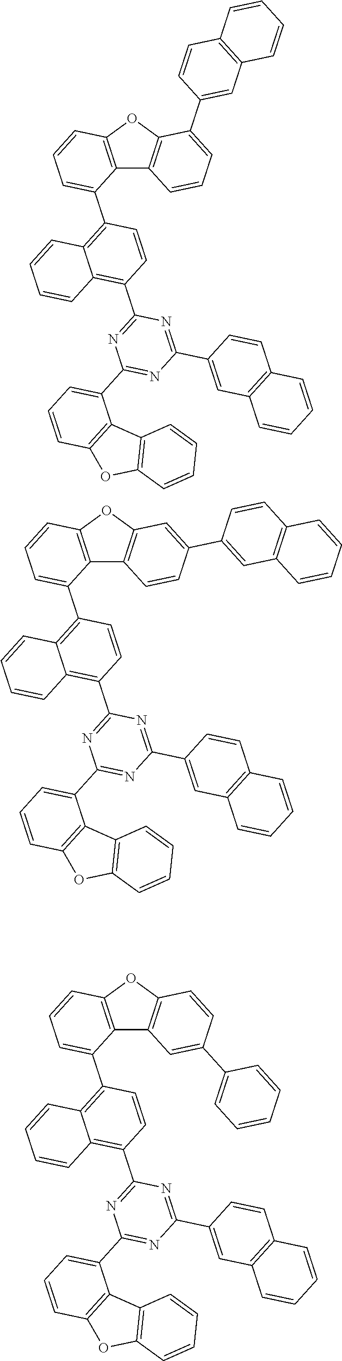

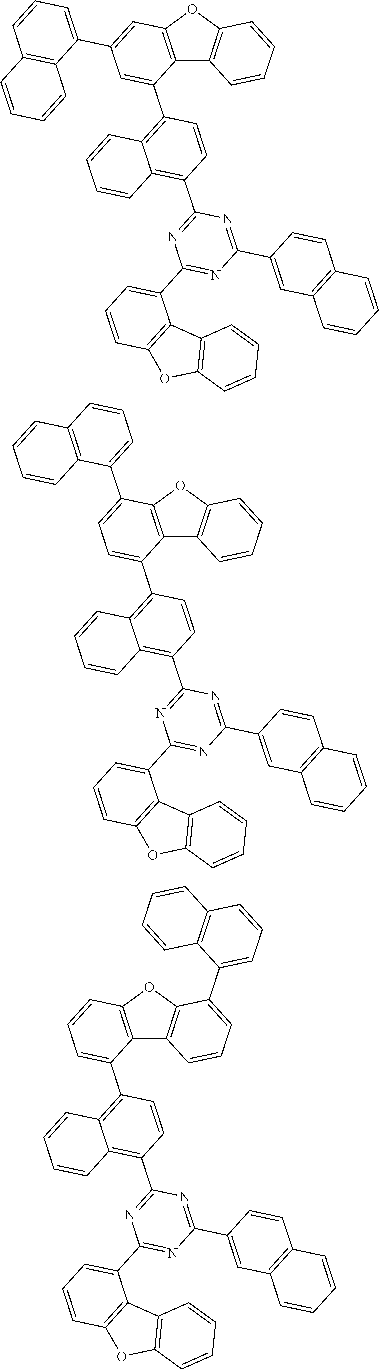

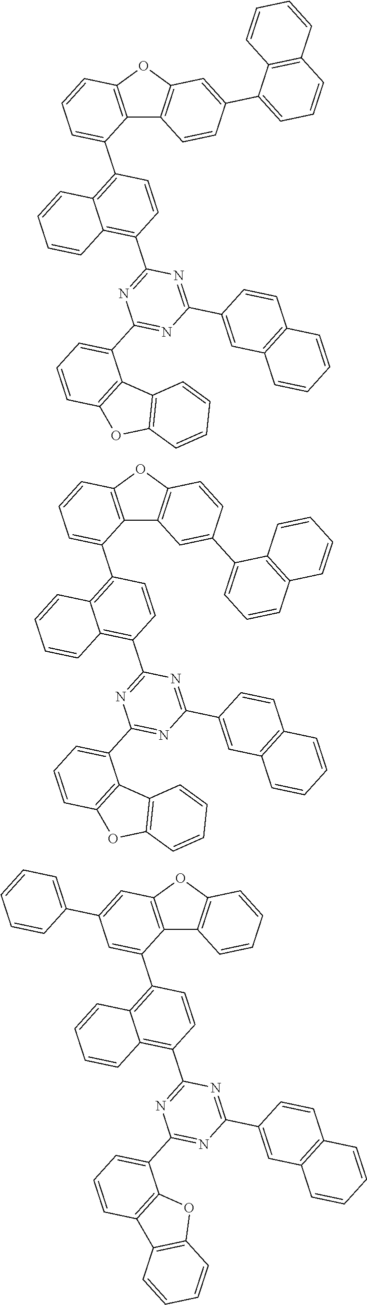

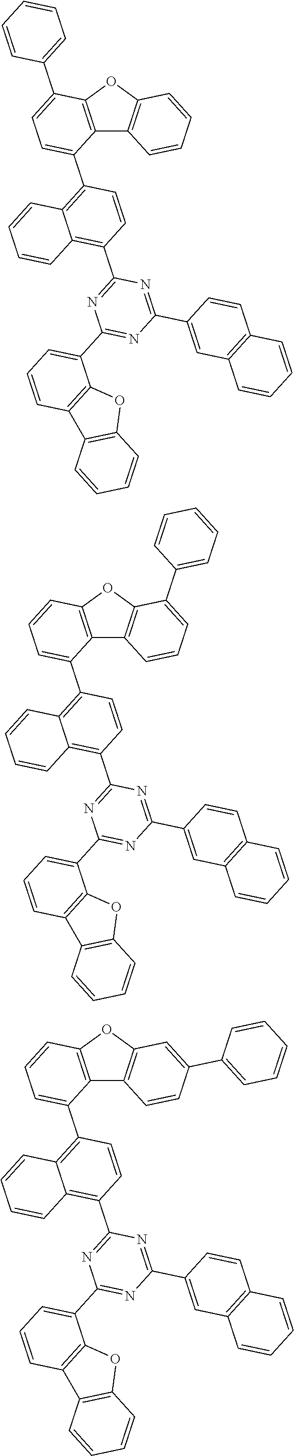

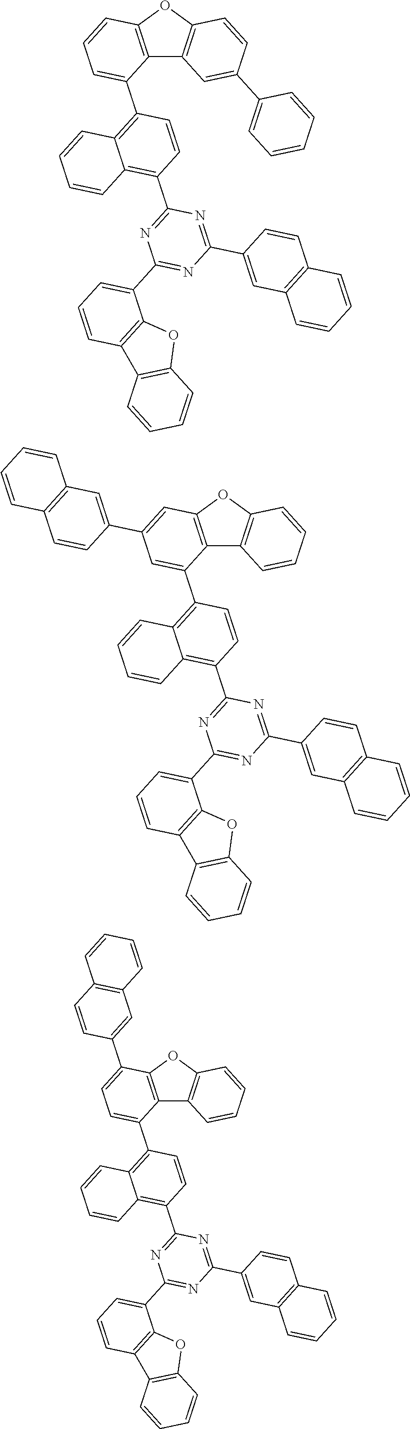 [SMILES: c1ccc(-c2ccc3oc4cccc(-c5ccc(-c6nc(-c7ccc8ccccc8c7)nc(-c7cccc8c7oc7ccccc78)n6)c6ccccc56)c4c3c2)cc1 ; c1ccc2cc(-c3cc(-c4ccc(-c5nc(-c6ccc7ccccc7c6)nc(-c6cccc7c6oc6ccccc67)n5)c5ccccc45)c4c(c3)oc3ccccc34)ccc2c1 ; c1ccc2cc(-c3nc(-c4ccc(-c5ccc(-c6ccc7ccccc7c6)c6oc7ccccc7c56)c5ccccc45)nc(-c4cccc5c4oc4ccccc45)n3)ccc2c1]

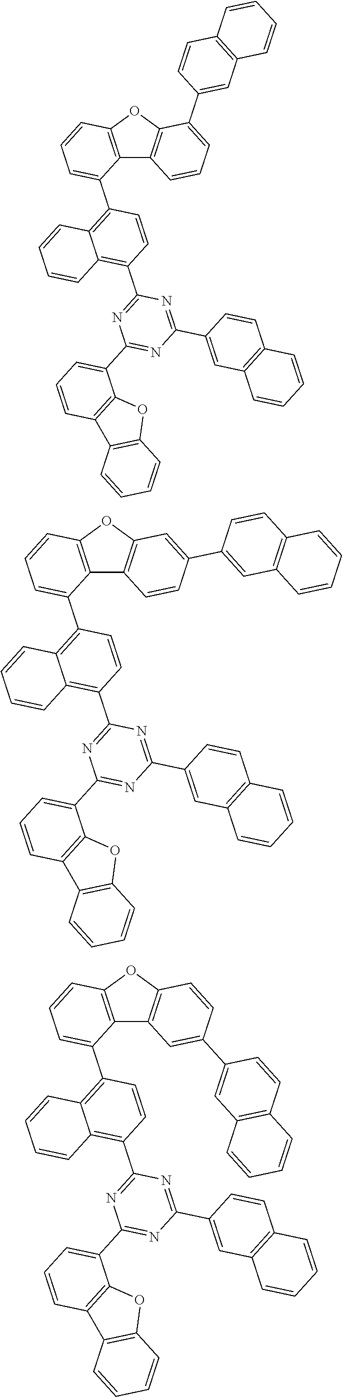

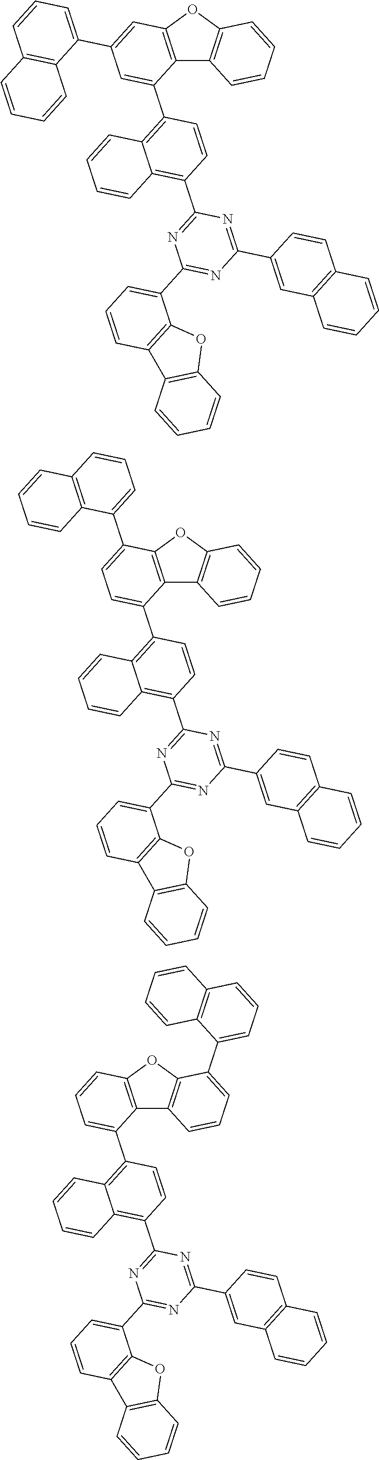

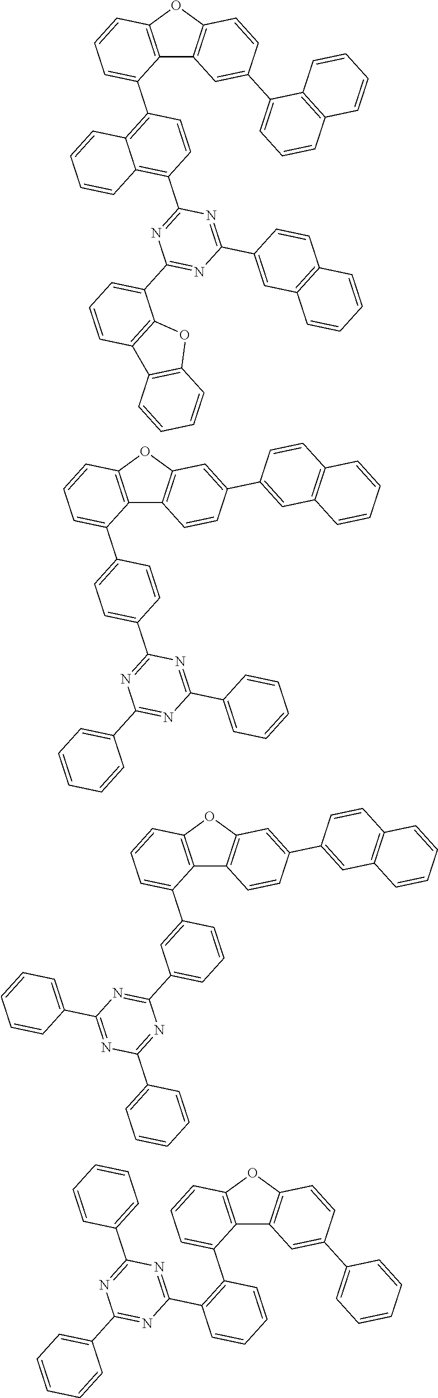

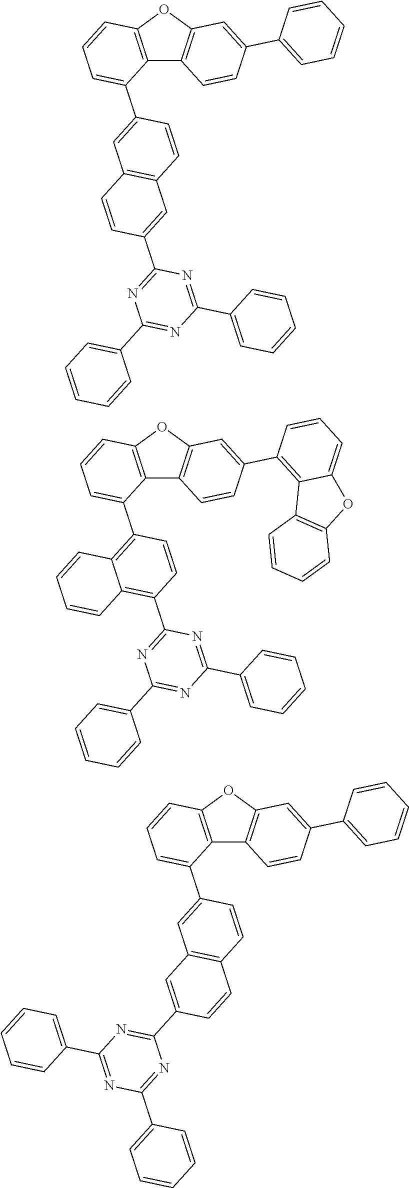

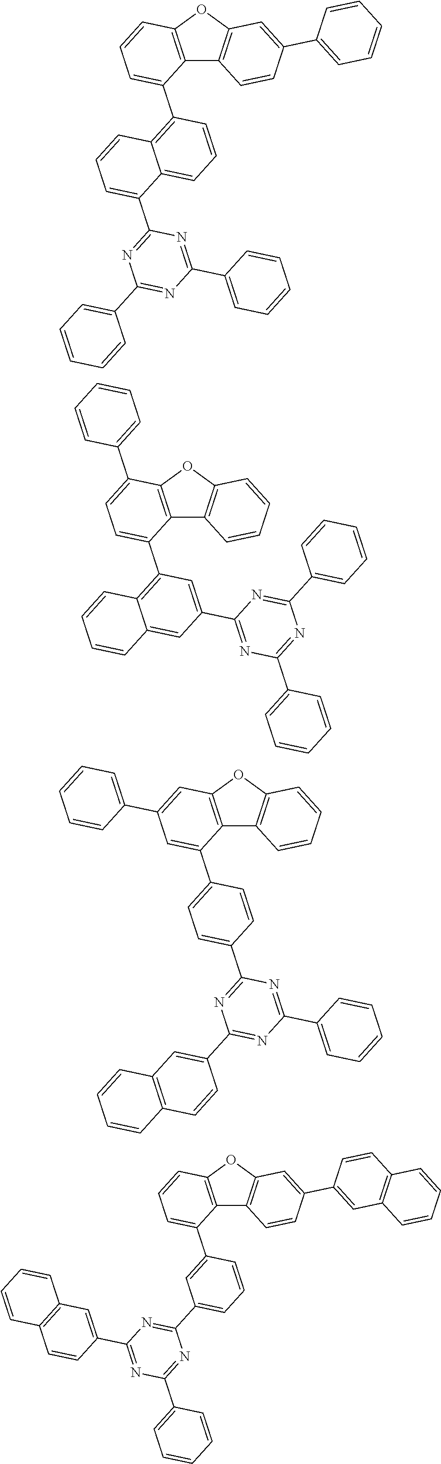

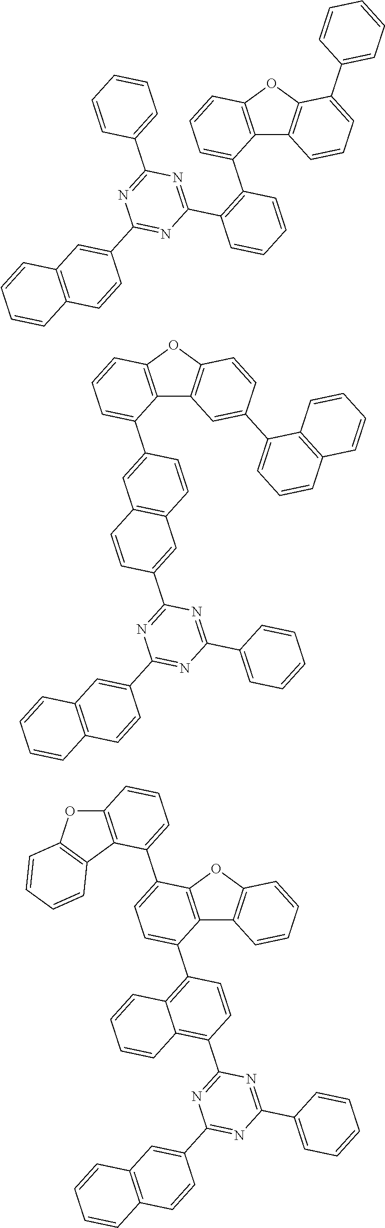

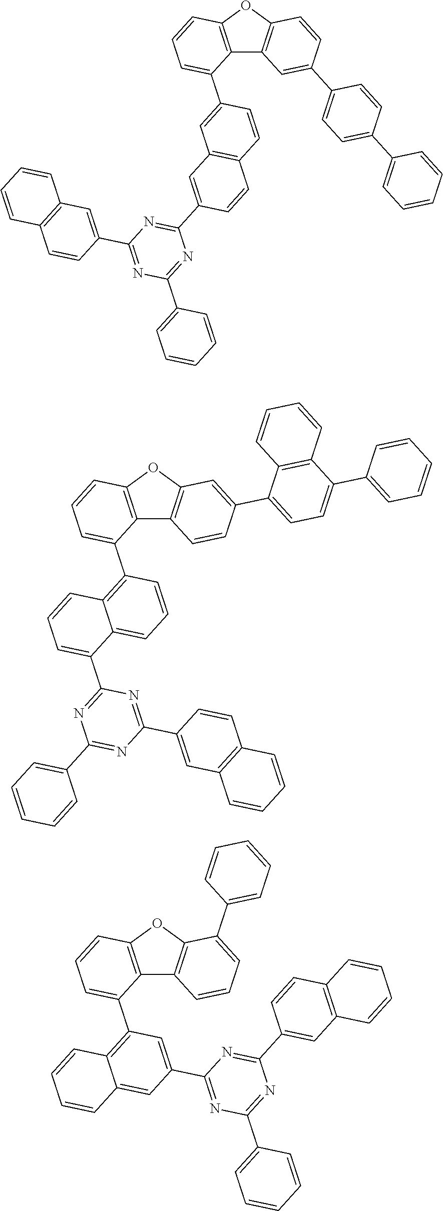

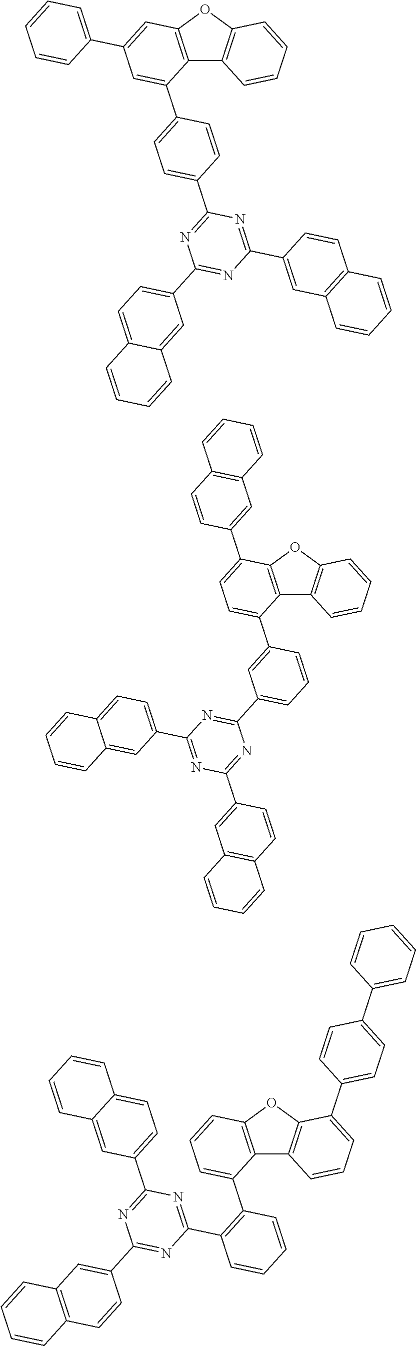 [SMILES: c1ccc(-c2cc(-c3ccc(-c4nc(-c5ccc6ccccc6c5)nc(-c5ccc6ccccc6c5)n4)cc3)c3c(c2)oc2ccccc23)cc1 ; c1cc(-c2nc(-c3ccc4ccccc4c3)nc(-c3ccc4ccccc4c3)n2)cc(-c2ccc(-c3ccc4ccccc4c3)c3oc4ccccc4c23)c1 ; c1ccc(-c2ccc(-c3cccc4c3oc3cccc(-c5ccccc5-c5nc(-c6ccc7ccccc7c6)nc(-c6ccc7ccccc7c6)n5)c34)cc2)cc1]

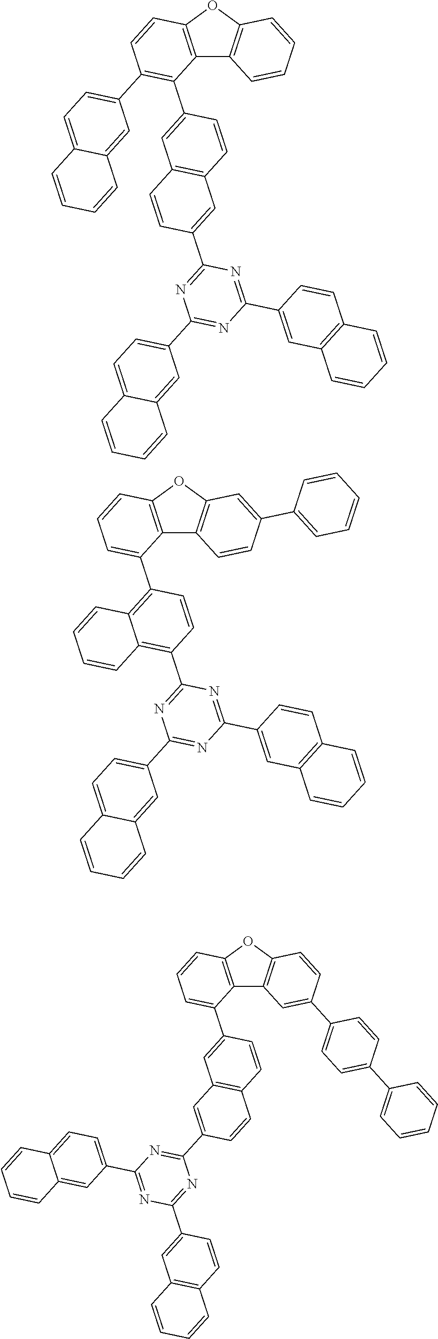

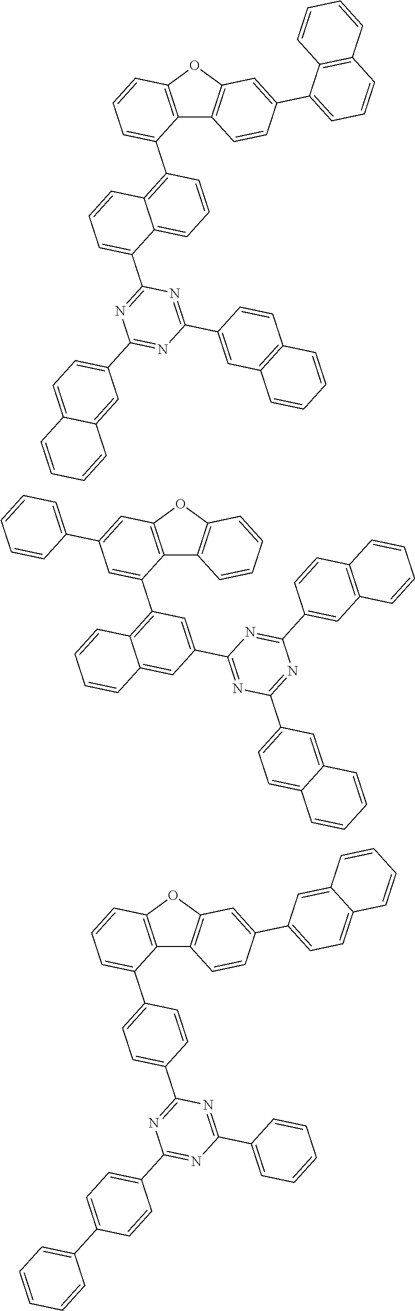

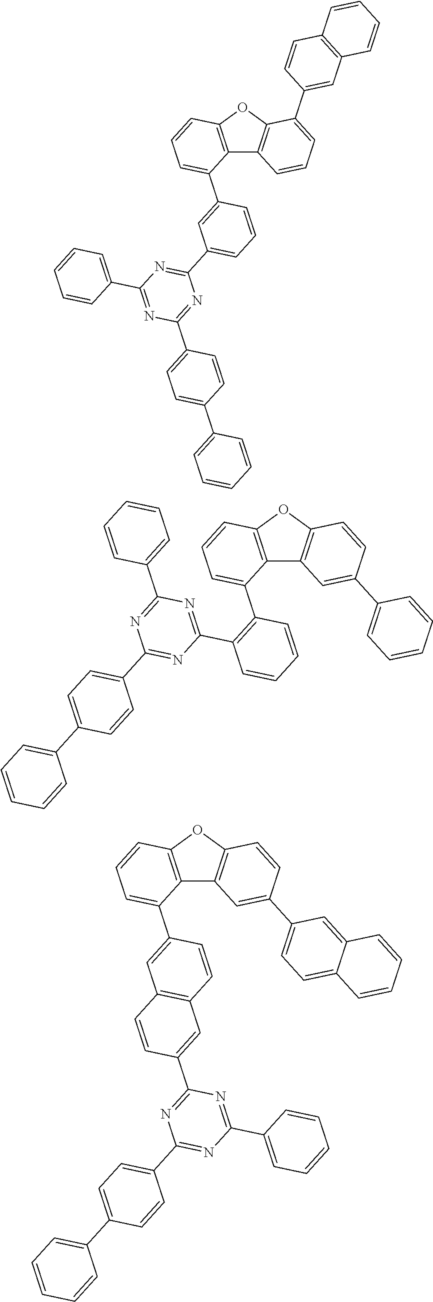

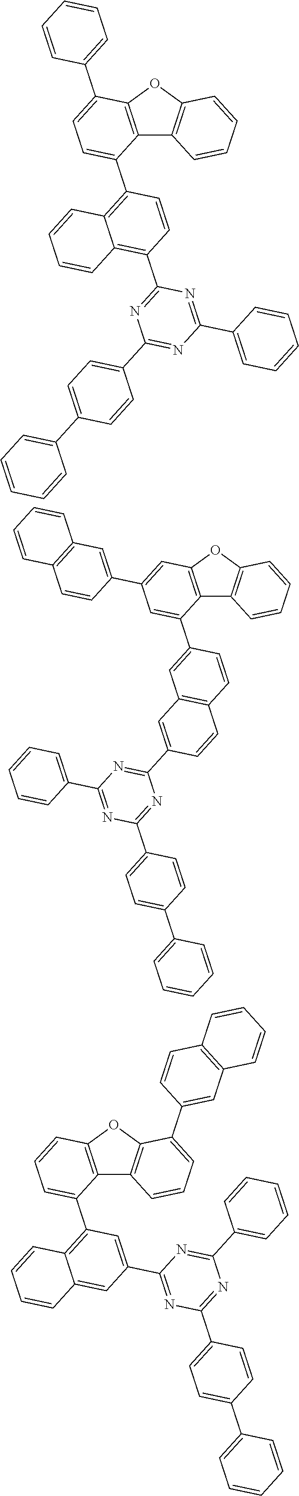

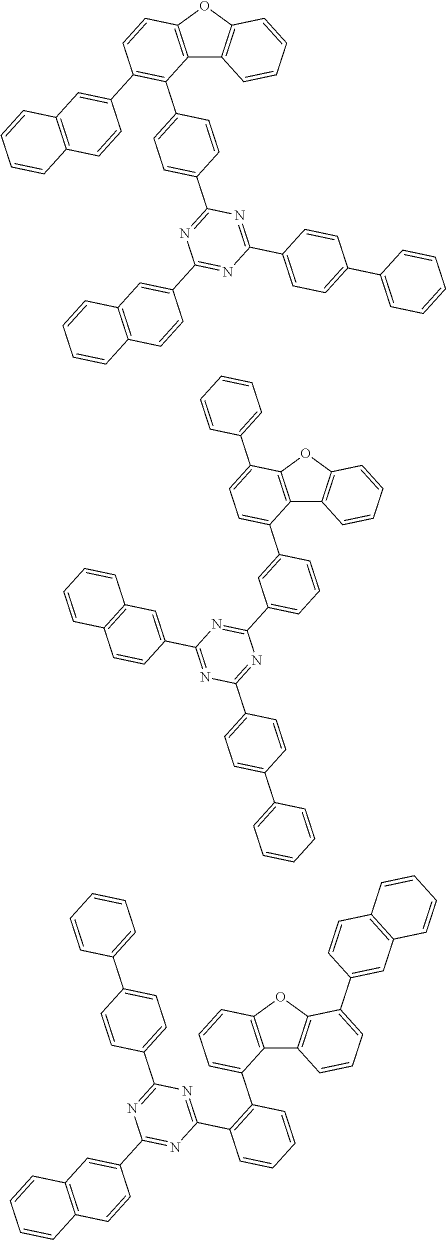 [SMILES: c1ccc(-c2ccc(-c3nc(-c4ccc(-c5c(-c6ccc7ccccc7c6)ccc6oc7ccccc7c56)cc4)nc(-c4ccc5ccccc5c4)n3)cc2)cc1 ; c1ccc(-c2ccc(-c3nc(-c4cccc(-c5ccc(-c6ccccc6)c6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6c(-c7ccc8ccccc8c7)cccc6c45)n3)cc2)cc1]

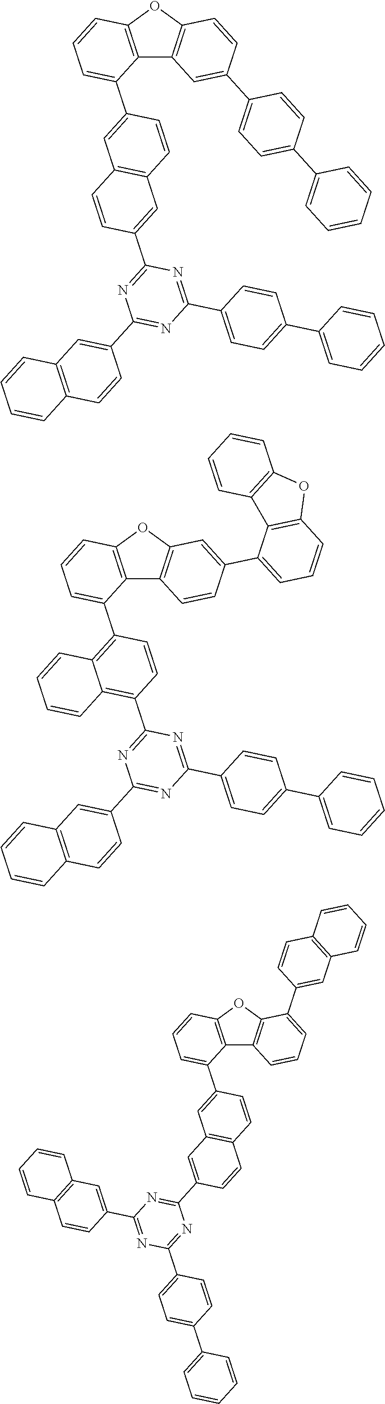 [SMILES: c1ccc(-c2ccc(-c3ccc4oc5cccc(-c6ccc7cc(-c8nc(-c9ccc(-c%10ccccc%10)cc9)nc(-c9ccc%10ccccc%10c9)n8)ccc7c6)c5c4c3)cc2)cc1 ; c1ccc(-c2ccc(-c3nc(-c4ccc5ccccc5c4)nc(-c4ccc(-c5cccc6oc7cc(-c8cccc9oc%10ccccc%10c89)ccc7c56)c5ccccc45)n3)cc2)cc1 ; c1ccc(-c2ccc(-c3nc(-c4ccc5ccccc5c4)nc(-c4ccc5ccc(-c6cccc7oc8c(-c9ccc%10ccccc%10c9)cccc8c67)cc5c4)n3)cc2)cc1]

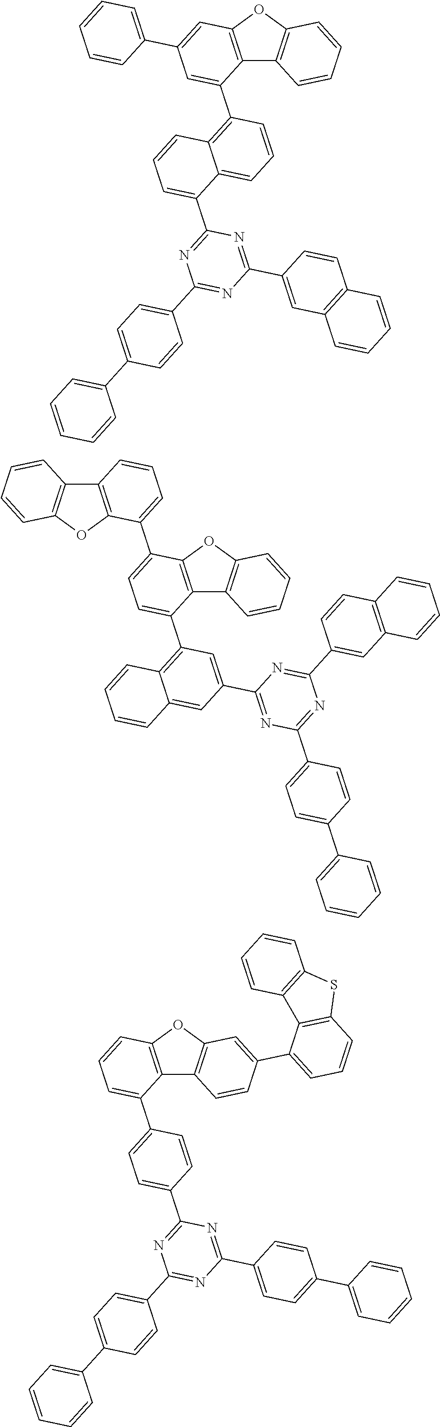

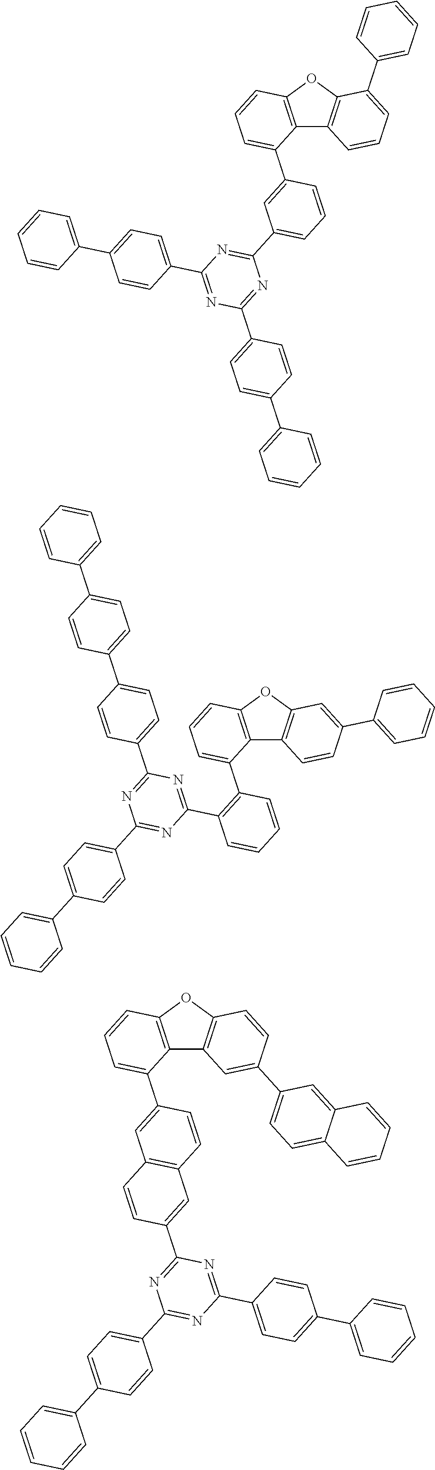 [SMILES: c1ccc(-c2ccc(-c3nc(-c4ccc(-c5ccccc5)cc4)nc(-c4cccc(-c5cccc6oc7c(-c8ccccc8)cccc7c56)c4)n3)cc2)cc1 ; c1ccc(-c2ccc(-c3ccc(-c4nc(-c5ccc(-c6ccccc6)cc5)nc(-c5ccccc5-c5cccc6oc7cc(-c8ccccc8)ccc7c56)n4)cc3)cc2)cc1 ; c1ccc(-c2ccc(-c3nc(-c4ccc(-c5ccccc5)cc4)nc(-c4ccc5cc(-c6cccc7oc8ccc(-c9ccc%10ccccc%10c9)cc8c67)ccc5c4)n3)cc2)cc1]

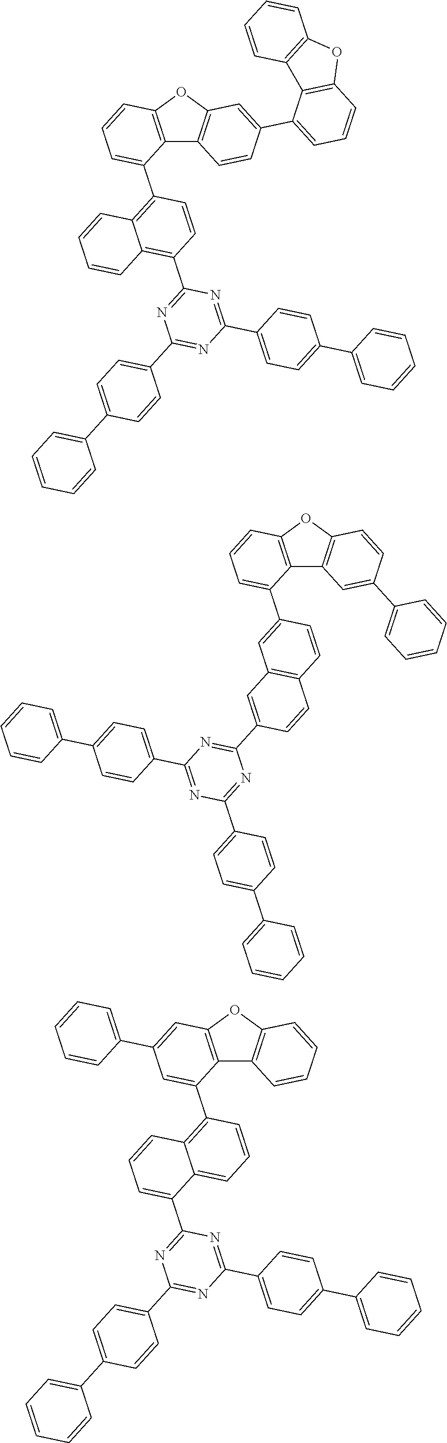

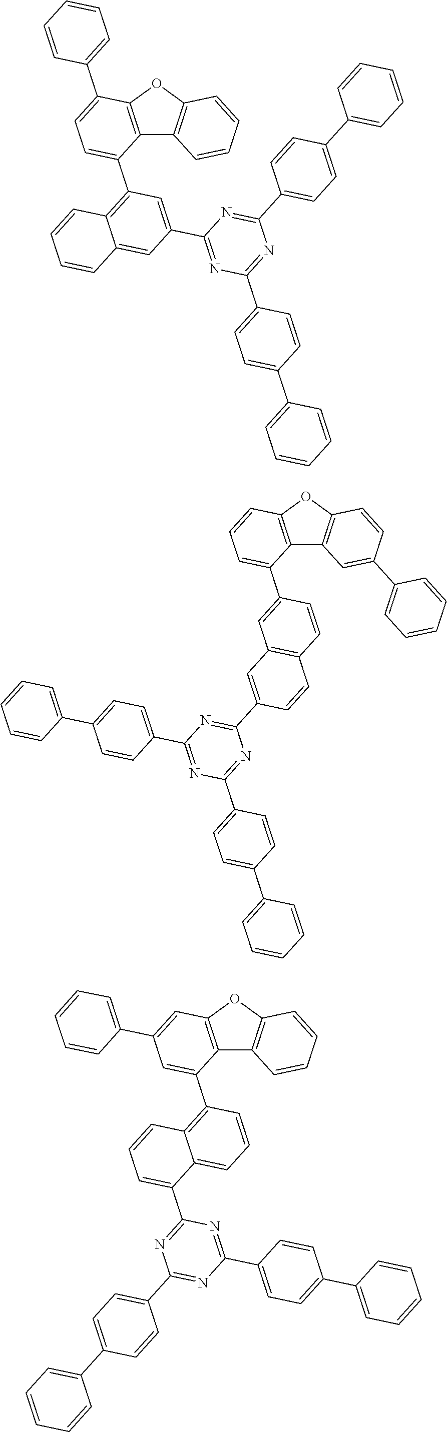

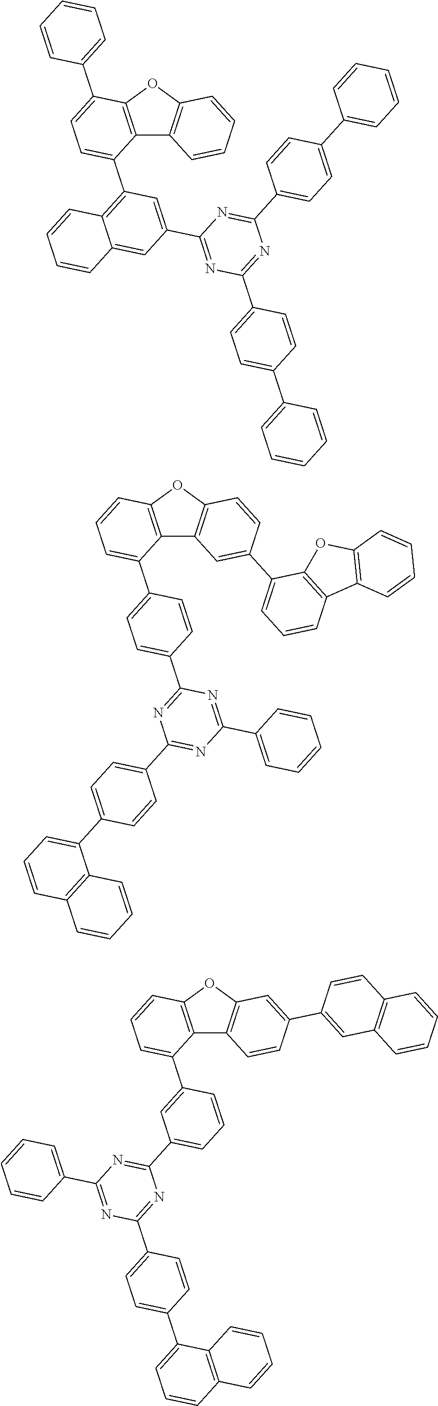 [SMILES: c1ccc(-c2ccc(-c3nc(-c4ccc(-c5ccccc5)cc4)nc(-c4cc(-c5ccc(-c6ccccc6)c6oc7ccccc7c56)c5ccccc5c4)n3)cc2)cc1 ; c1ccc(-c2nc(-c3ccc(-c4cccc5ccccc45)cc3)nc(-c3ccc(-c4cccc5oc6ccc(-c7cccc8c7oc7ccccc78)cc6c45)cc3)n2)cc1 ; c1ccc(-c2nc(-c3ccc(-c4cccc5ccccc45)cc3)nc(-c3cccc(-c4cccc5oc6cc(-c7ccc8ccccc8c7)ccc6c45)c3)n2)cc1]

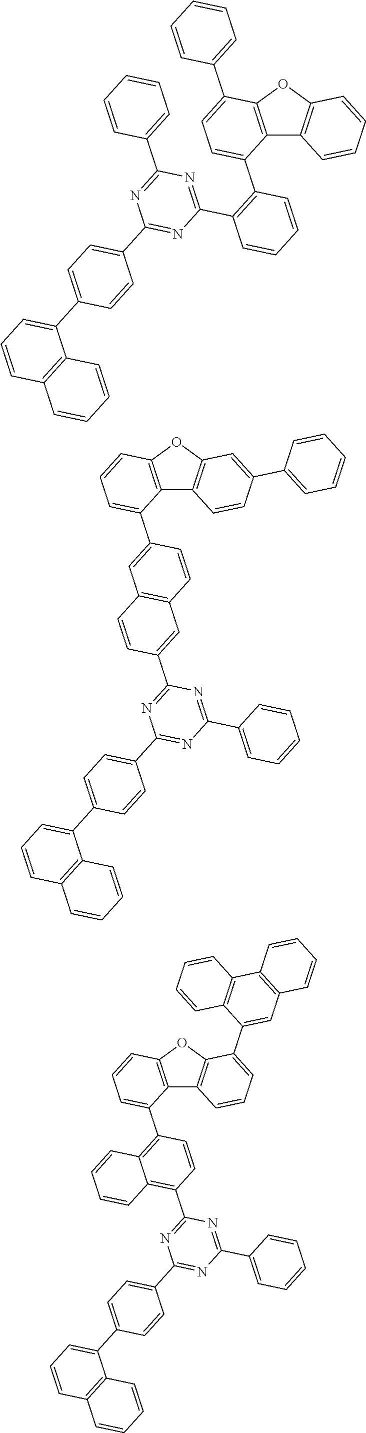 [SMILES: c1ccc(-c2nc(-c3ccc(-c4cccc5ccccc45)cc3)nc(-c3ccccc3-c3ccc(-c4ccccc4)c4oc5ccccc5c34)n2)cc1 ; c1ccc(-c2ccc3c(c2)oc2cccc(-c4ccc5cc(-c6nc(-c7ccccc7)nc(-c7ccc(-c8cccc9ccccc89)cc7)n6)ccc5c4)c23)cc1 ; c1ccc(-c2nc(-c3ccc(-c4cccc5ccccc45)cc3)nc(-c3ccc(-c4cccc5oc6c(-c7cc8ccccc8c8ccccc78)cccc6c45)c4ccccc34)n2)cc1]

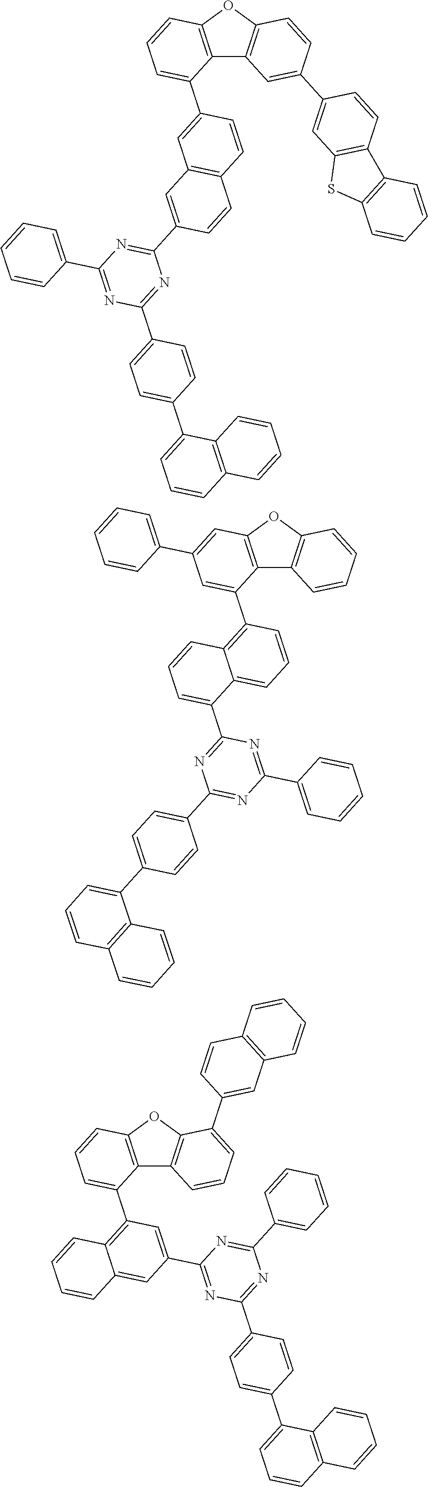

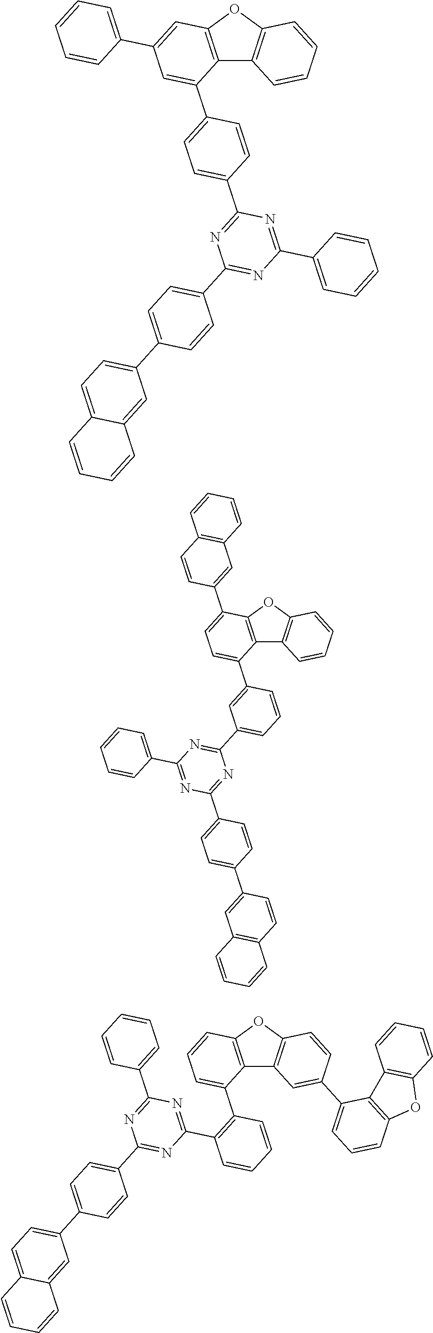

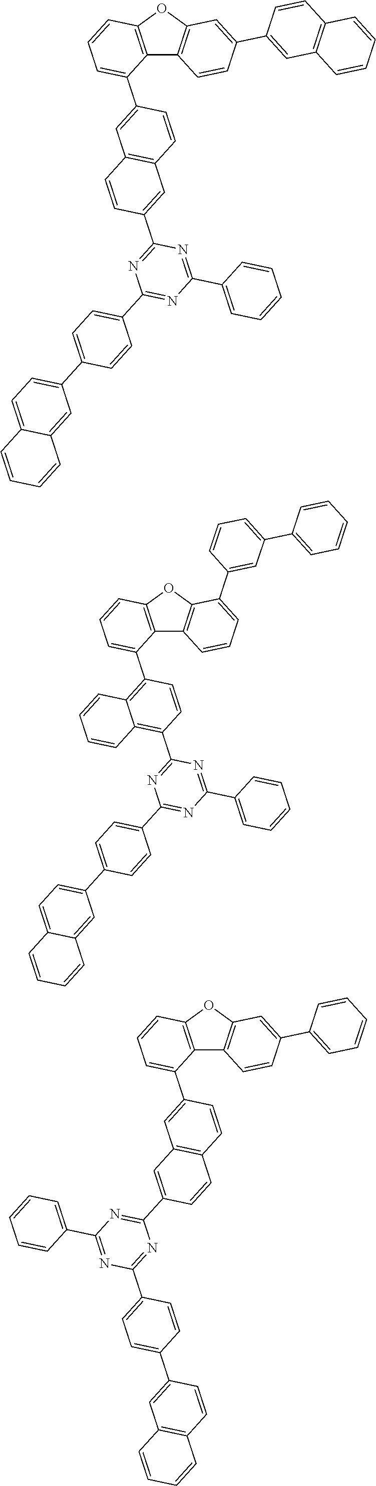 [SMILES: c1ccc(-c2nc(-c3ccc(-c4ccc5ccccc5c4)cc3)nc(-c3ccc4cc(-c5cccc6oc7cc(-c8ccc9ccccc9c8)ccc7c56)ccc4c3)n2)cc1 ; c1ccc(-c2cccc(-c3cccc4c3oc3cccc(-c5ccc(-c6nc(-c7ccccc7)nc(-c7ccc(-c8ccc9ccccc9c8)cc7)n6)c6ccccc56)c34)c2)cc1 ; c1ccc(-c2ccc3c(c2)oc2cccc(-c4ccc5ccc(-c6nc(-c7ccccc7)nc(-c7ccc(-c8ccc9ccccc9c8)cc7)n6)cc5c4)c23)cc1]

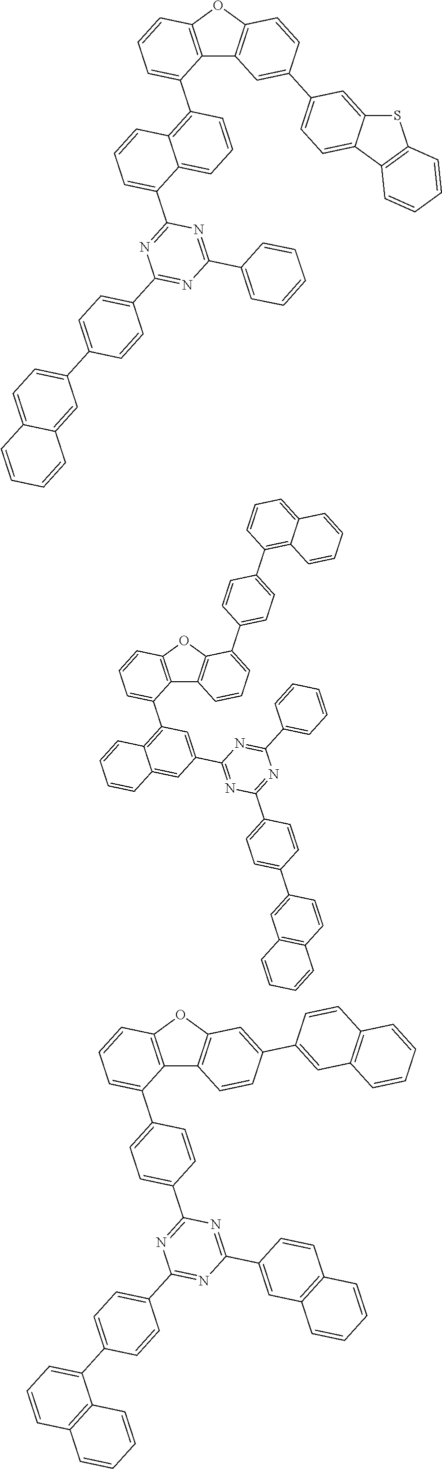

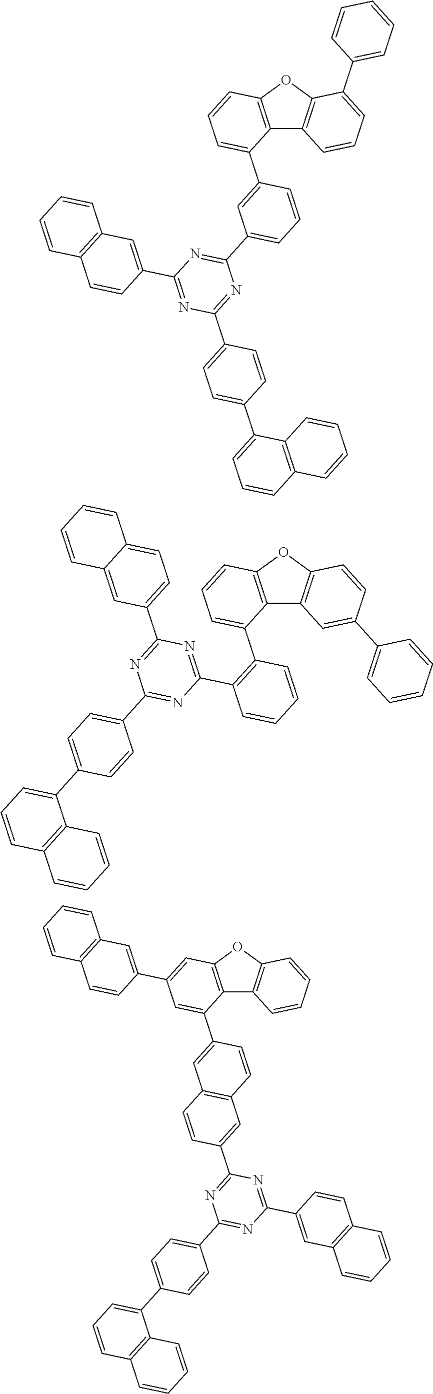

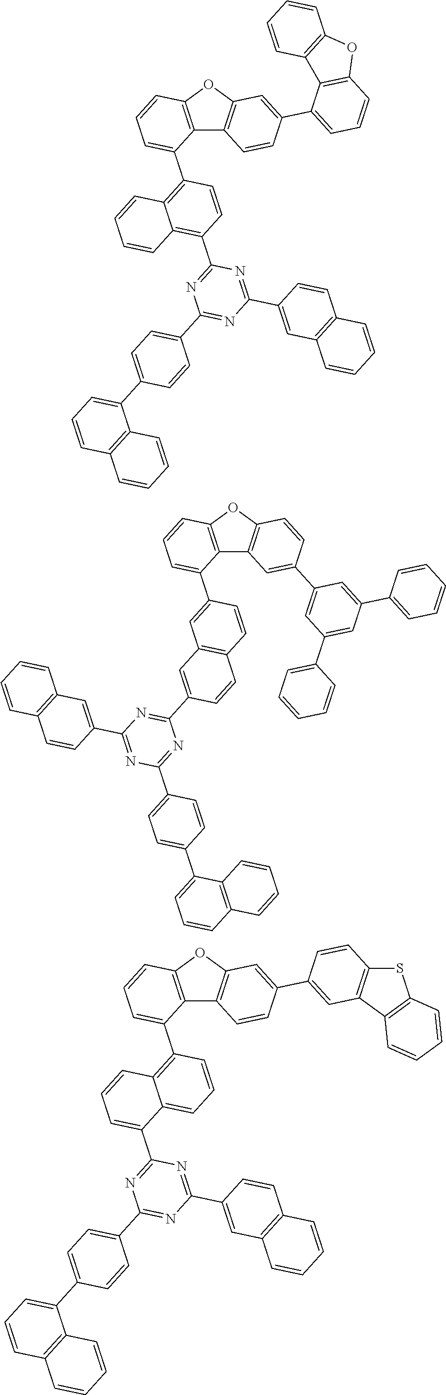

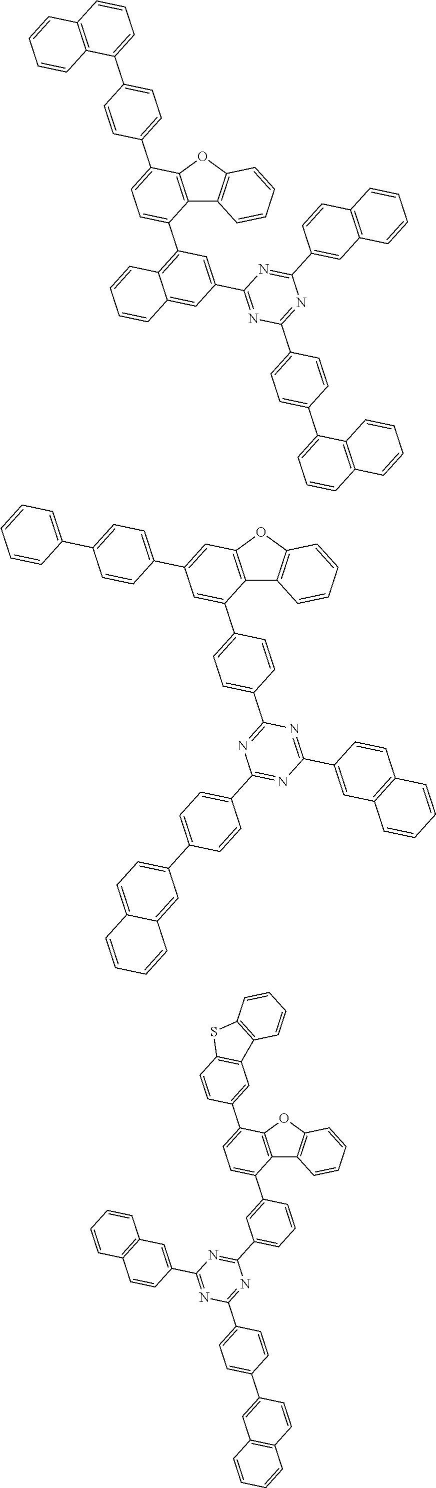 [SMILES: c1ccc2cc(-c3nc(-c4ccc(-c5cccc6ccccc56)cc4)nc(-c4cc(-c5ccc(-c6ccc(-c7cccc8ccccc78)cc6)c6oc7ccccc7c56)c5ccccc5c4)n3)ccc2c1 ; c1ccc(-c2ccc(-c3cc(-c4ccc(-c5nc(-c6ccc(-c7ccc8ccccc8c7)cc6)nc(-c6ccc7ccccc7c6)n5)cc4)c4c(c3)oc3ccccc34)cc2)cc1 ; c1cc(-c2nc(-c3ccc(-c4ccc5ccccc5c4)cc3)nc(-c3ccc4ccccc4c3)n2)cc(-c2ccc(-c3ccc4sc5ccccc5c4c3)c3oc4ccccc4c23)c1]

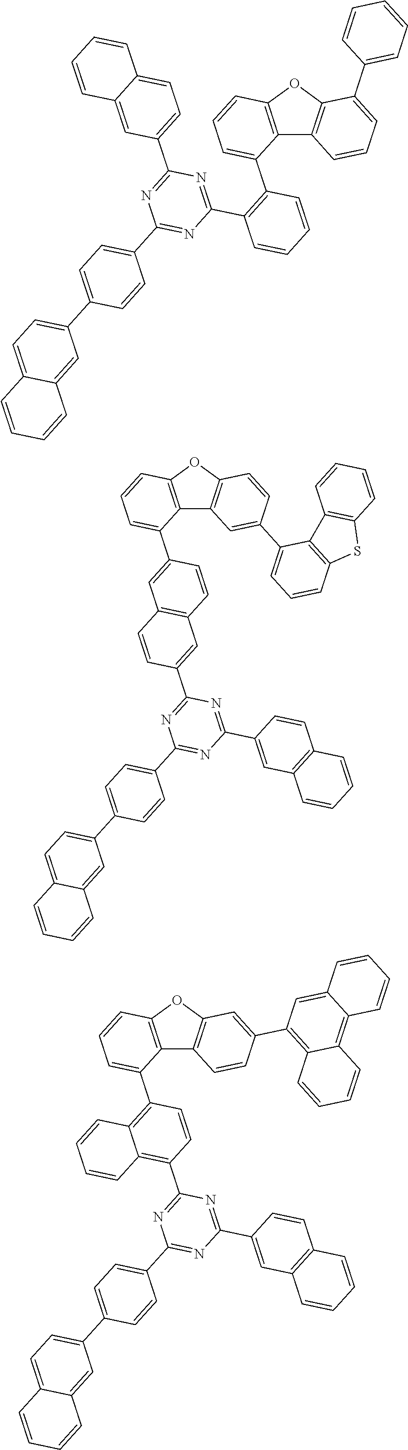 [SMILES: c1ccc(-c2cccc3c2oc2cccc(-c4ccccc4-c4nc(-c5ccc(-c6ccc7ccccc7c6)cc5)nc(-c5ccc6ccccc6c5)n4)c23)cc1 ; c1ccc2cc(-c3ccc(-c4nc(-c5ccc6ccccc6c5)nc(-c5ccc6cc(-c7cccc8oc9ccc(-c%10cccc%11sc%12ccccc%12c%10%11)cc9c78)ccc6c5)n4)cc3)ccc2c1 ; c1ccc2cc(-c3ccc(-c4nc(-c5ccc6ccccc6c5)nc(-c5ccc(-c6cccc7oc8cc(-c9cc%10ccccc%10c%10ccccc9%10)ccc8c67)c6ccccc56)n4)cc3)ccc2c1]

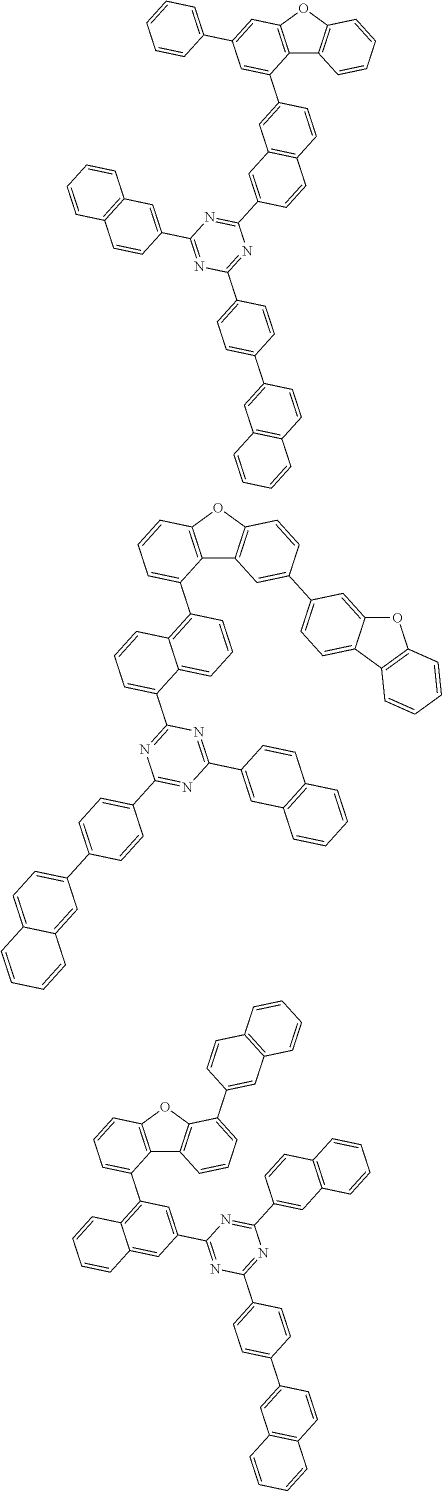

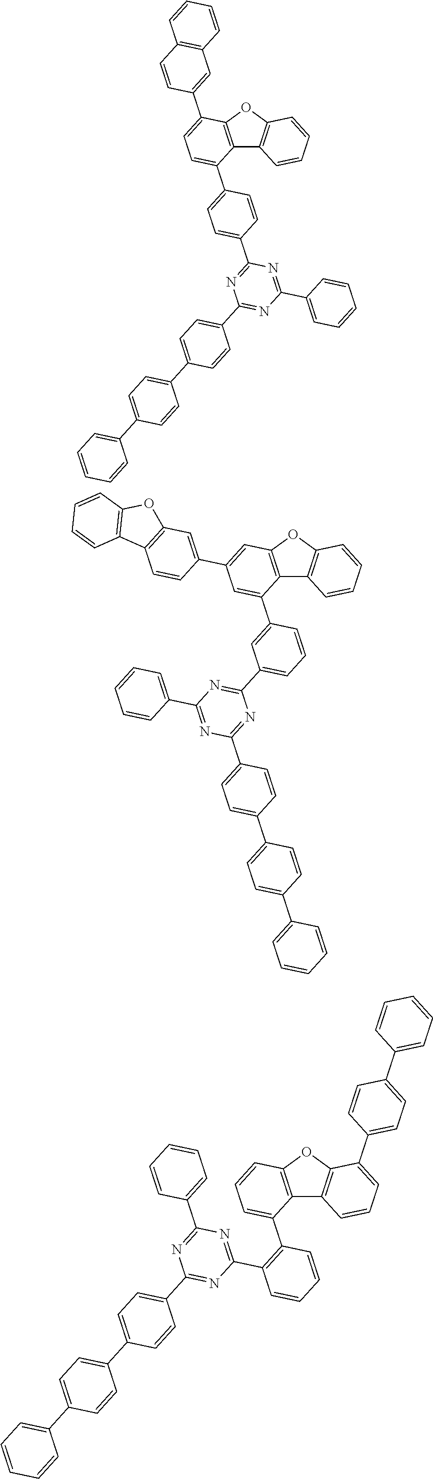

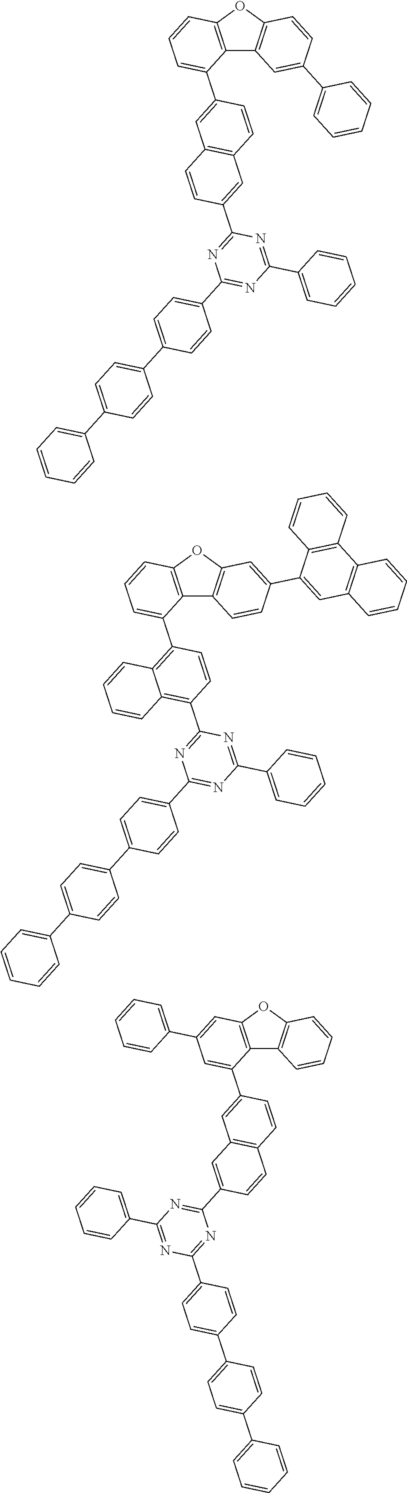 [SMILES: c1ccc(-c2ccc(-c3ccc(-c4nc(-c5ccccc5)nc(-c5ccc6cc(-c7cccc8oc9ccc(-c%10ccccc%10)cc9c78)ccc6c5)n4)cc3)cc2)cc1 ; c1ccc(-c2ccc(-c3ccc(-c4nc(-c5ccccc5)nc(-c5ccc(-c6cccc7oc8cc(-c9cc%10ccccc%10c%10ccccc9%10)ccc8c67)c6ccccc56)n4)cc3)cc2)cc1 ; c1ccc(-c2ccc(-c3ccc(-c4nc(-c5ccccc5)nc(-c5ccc6ccc(-c7cc(-c8ccccc8)cc8oc9ccccc9c78)cc6c5)n4)cc3)cc2)cc1]

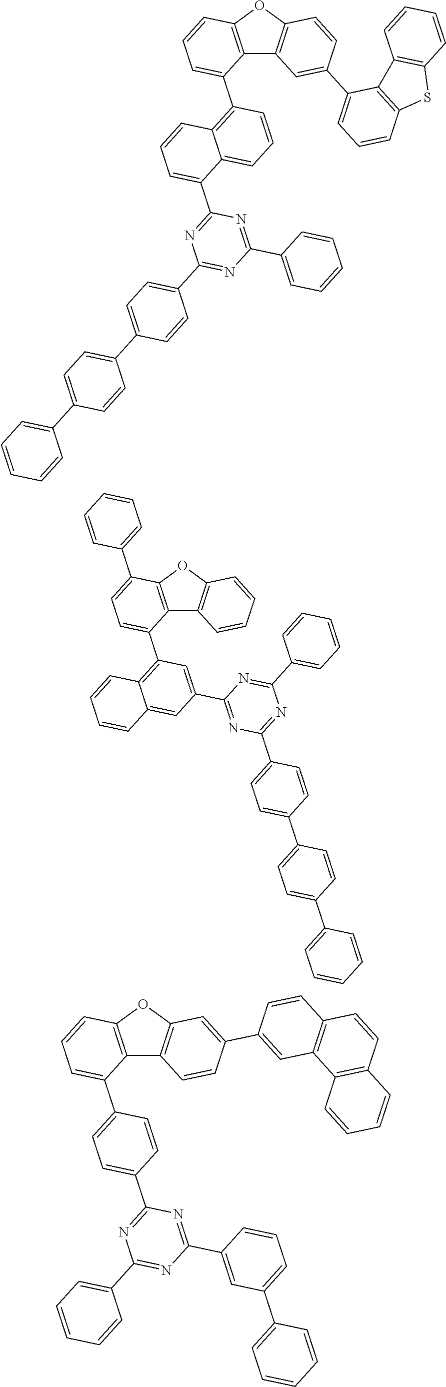

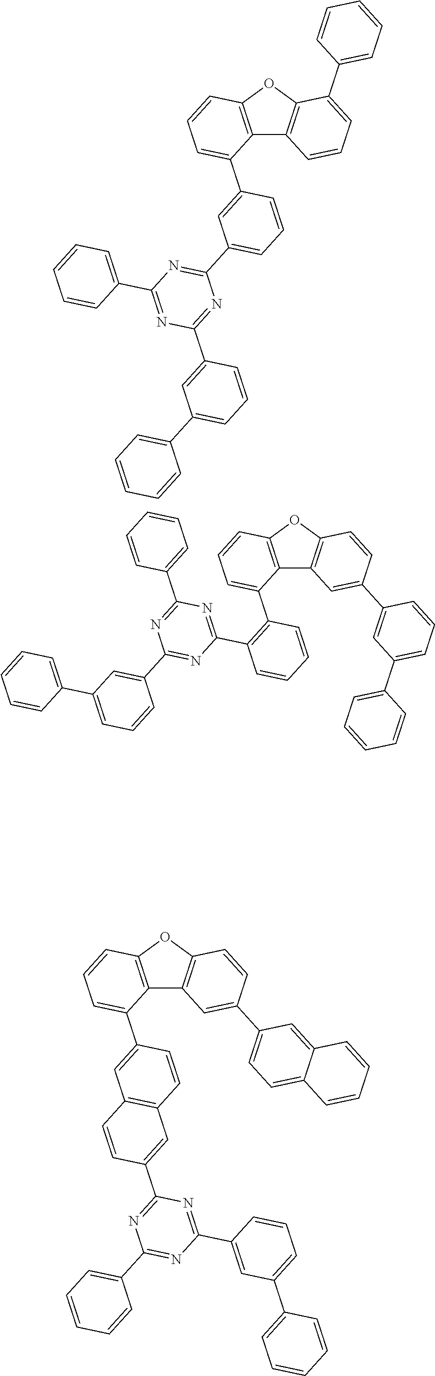

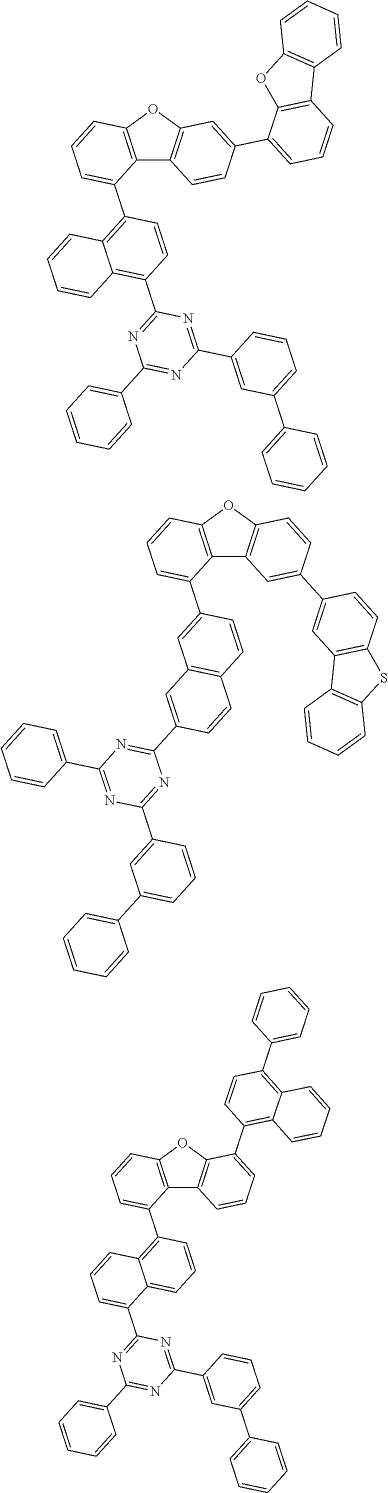

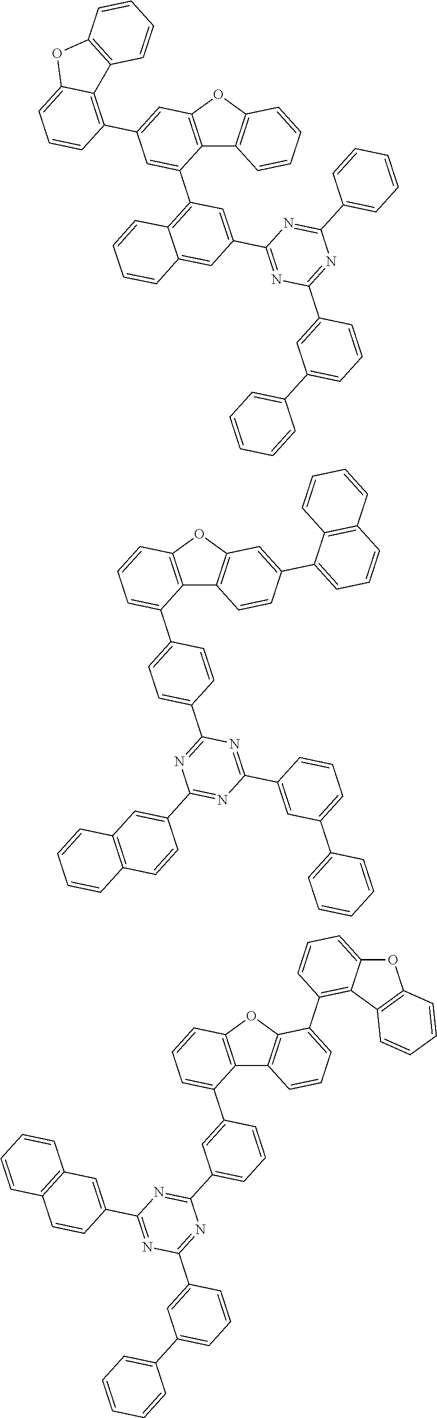

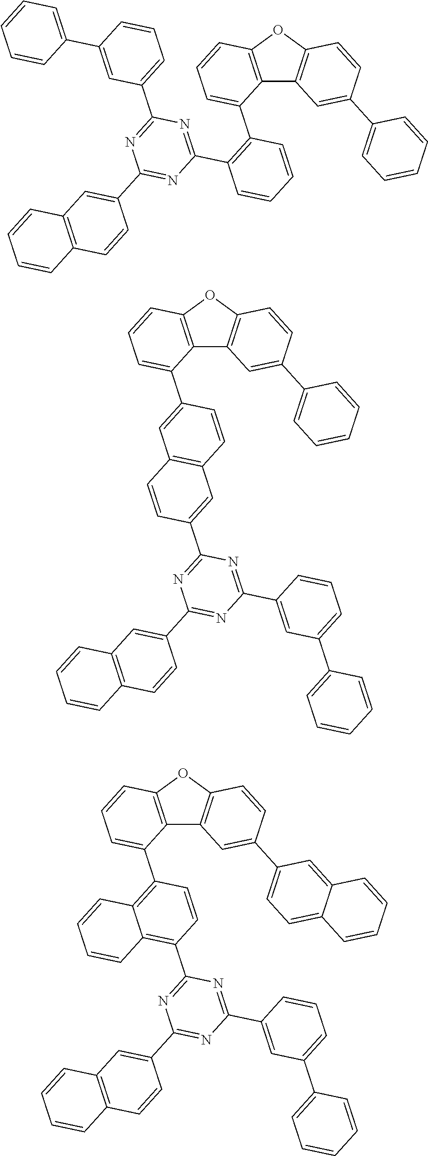 [SMILES: c1ccc(-c2cccc(-c3nc(-c4ccc5ccccc5c4)nc(-c4ccccc4-c4cccc5oc6ccc(-c7ccccc7)cc6c45)n3)c2)cc1 ; c1ccc(-c2cccc(-c3nc(-c4ccc5ccccc5c4)nc(-c4ccc5cc(-c6cccc7oc8ccc(-c9ccccc9)cc8c67)ccc5c4)n3)c2)cc1 ; c1ccc(-c2cccc(-c3nc(-c4ccc5ccccc5c4)nc(-c4ccc(-c5cccc6oc7ccc(-c8ccc9ccccc9c8)cc7c56)c5ccccc45)n3)c2)cc1]

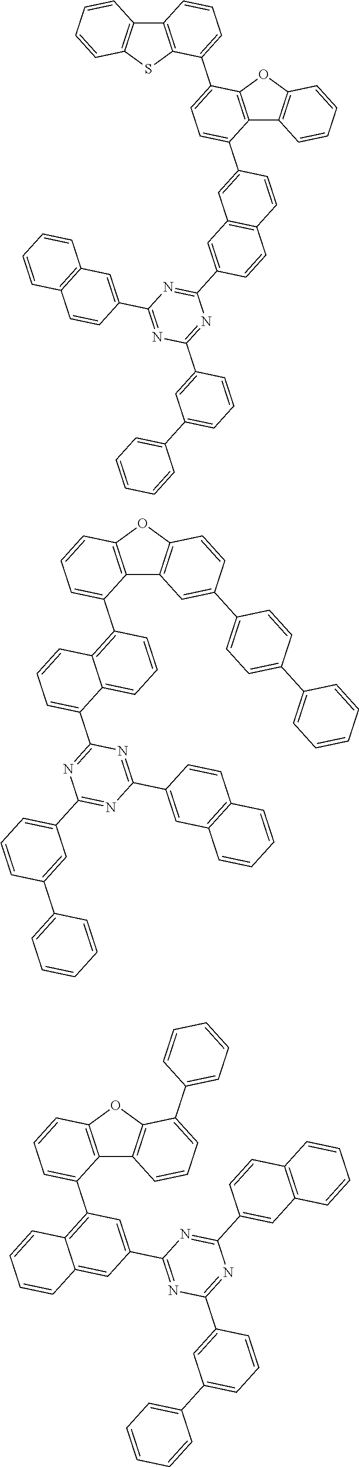

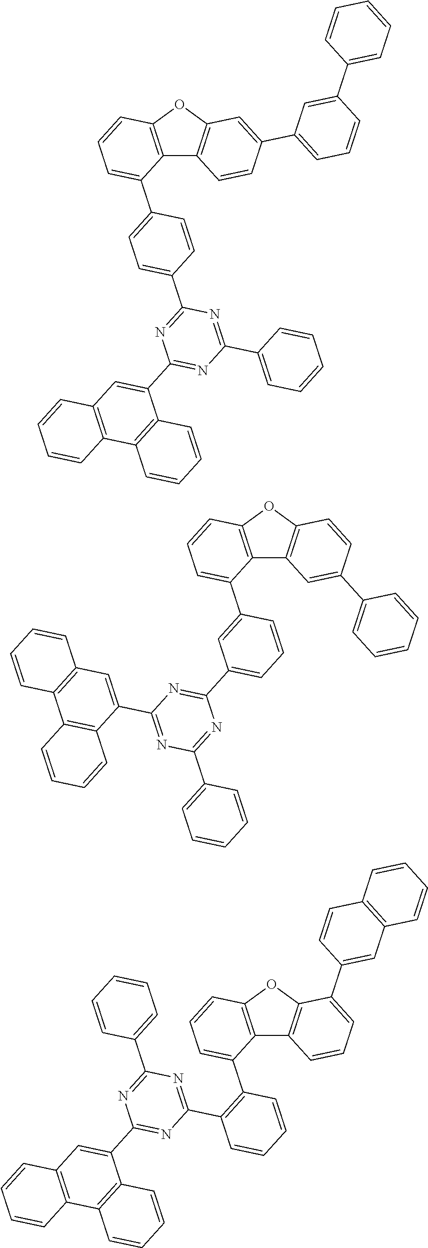 [SMILES: c1ccc(-c2cccc(-c3ccc4c(c3)oc3cccc(-c5ccc(-c6nc(-c7ccccc7)nc(-c7cc8ccccc8c8ccccc78)n6)cc5)c34)c2)cc1 ; c1ccc(-c2ccc3oc4cccc(-c5cccc(-c6nc(-c7ccccc7)nc(-c7cc8ccccc8c8ccccc78)n6)c5)c4c3c2)cc1 ; c1ccc(-c2nc(-c3ccccc3-c3cccc4oc5c(-c6ccc7ccccc7c6)cccc5c34)nc(-c3cc4ccccc4c4ccccc34)n2)cc1]

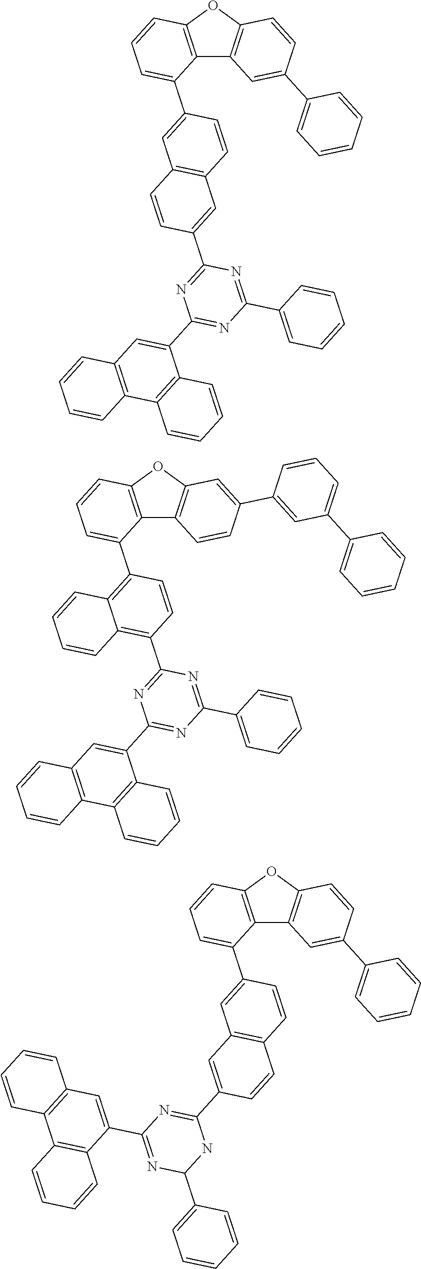

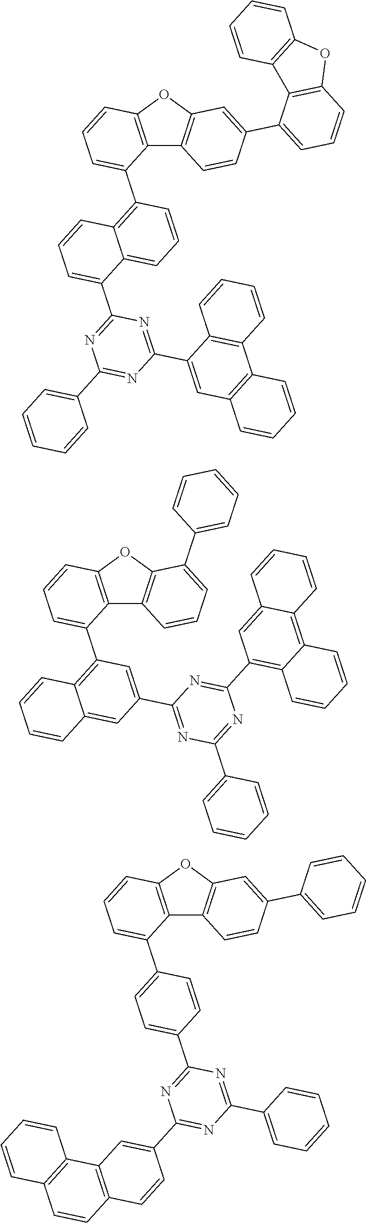

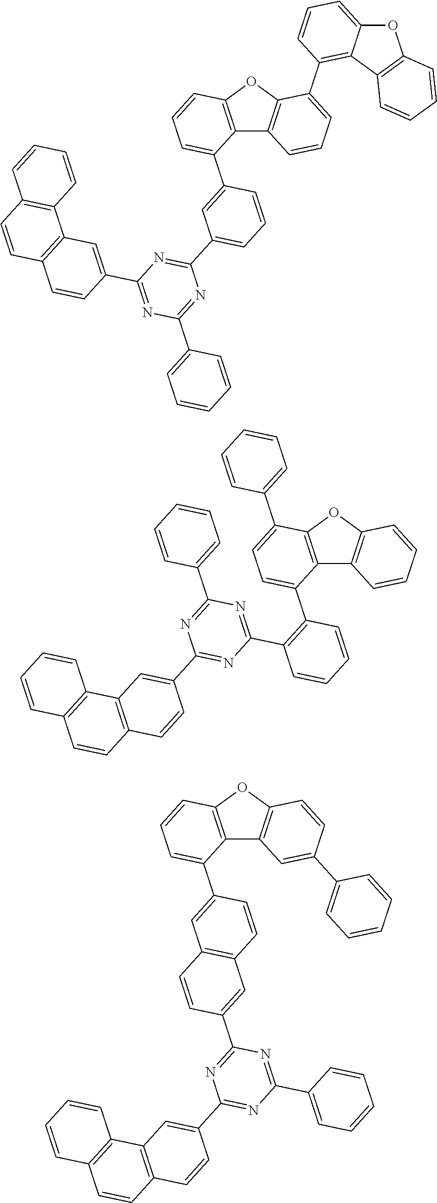

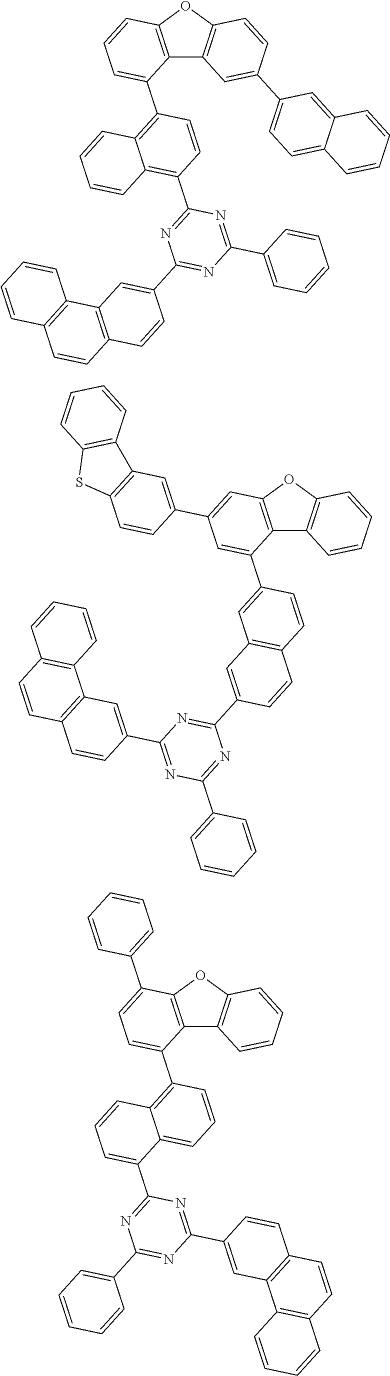

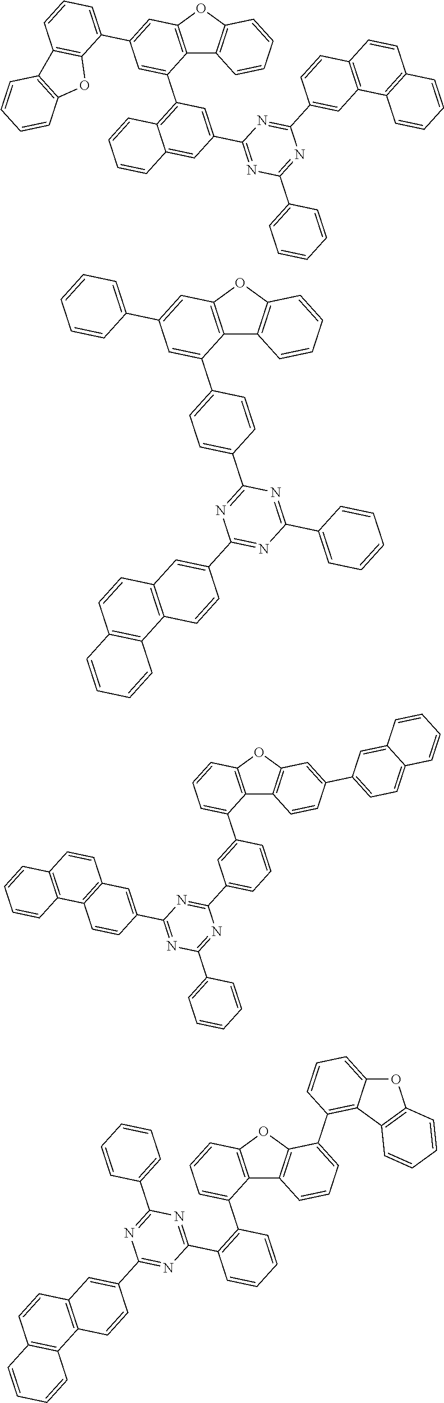

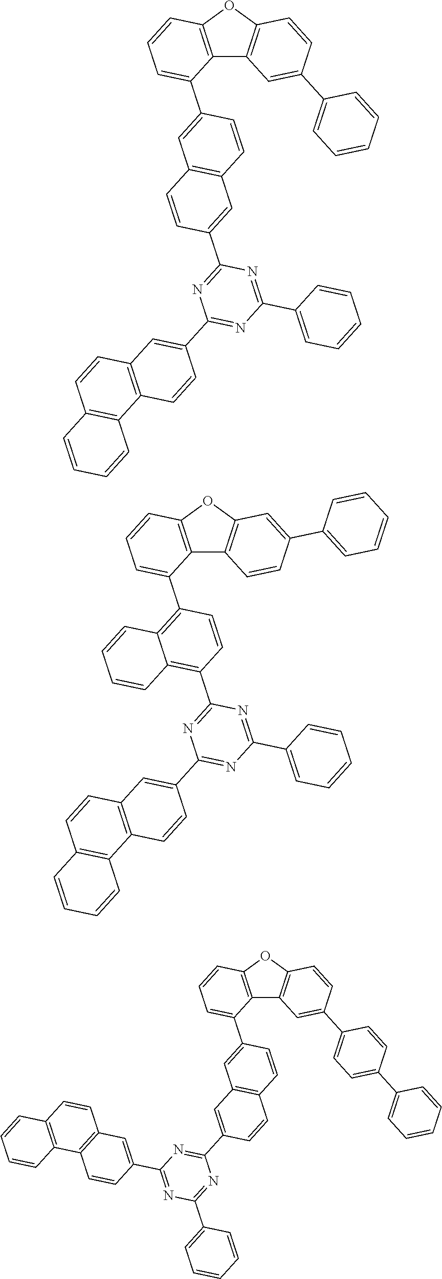 [SMILES: c1ccc(-c2ccc3oc4cccc(-c5ccc6cc(-c7nc(-c8ccccc8)nc(-c8ccc9c(ccc%10ccccc%109)c8)n7)ccc6c5)c4c3c2)cc1 ; c1ccc(-c2ccc3c(c2)oc2cccc(-c4ccc(-c5nc(-c6ccccc6)nc(-c6ccc7c(ccc8ccccc87)c6)n5)c5ccccc45)c23)cc1 ; c1ccc(-c2ccc(-c3ccc4oc5cccc(-c6ccc7ccc(-c8nc(-c9ccccc9)nc(-c9ccc%10c(ccc%11ccccc%11%10)c9)n8)cc7c6)c5c4c3)cc2)cc1]

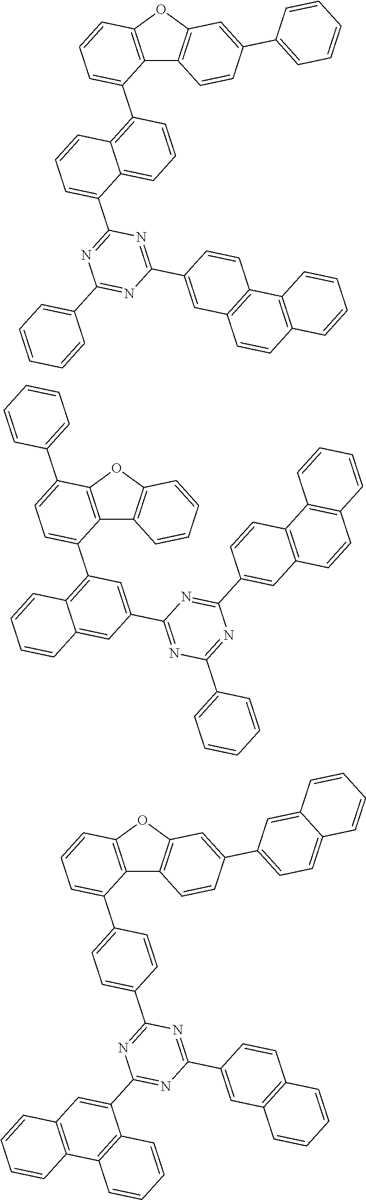

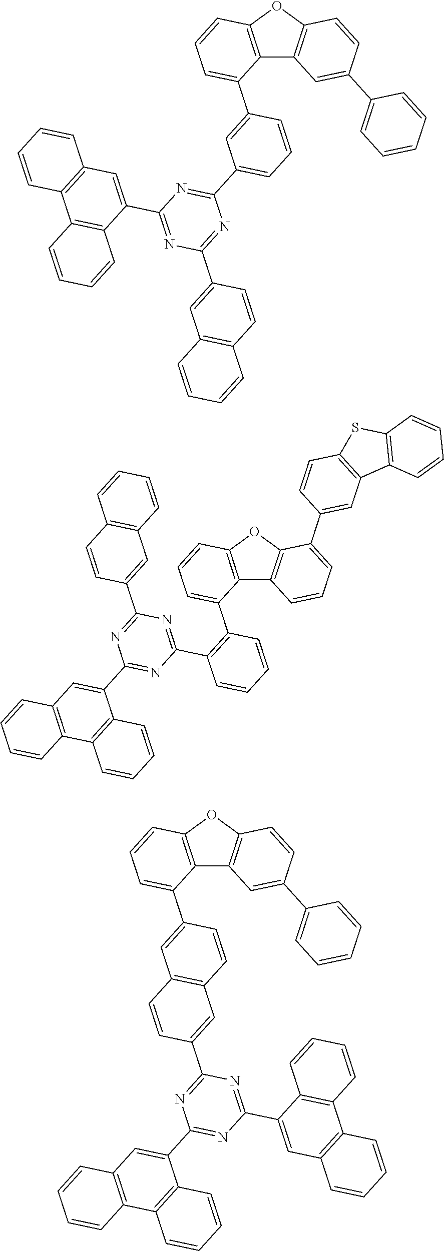 [SMILES: c1ccc(-c2ccc3oc4cccc(-c5cccc(-c6nc(-c7ccc8ccccc8c7)nc(-c7cc8ccccc8c8ccccc78)n6)c5)c4c3c2)cc1 ; c1ccc(-c2cccc3oc4c(-c5ccc6sc7ccccc7c6c5)cccc4c23)c(-c2nc(-c3ccc4ccccc4c3)nc(-c3cc4ccccc4c4ccccc34)n2)c1 ; c1ccc(-c2ccc3oc4cccc(-c5ccc6cc(-c7nc(-c8cc9ccccc9c9ccccc89)nc(-c8cc9ccccc9c9ccccc89)n7)ccc6c5)c4c3c2)cc1]

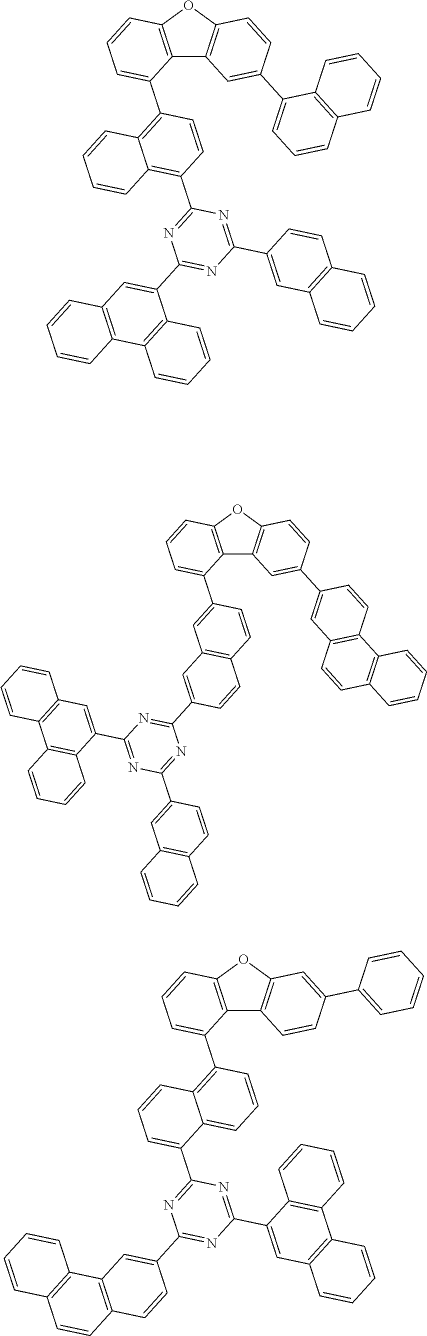 [SMILES: c1ccc2cc(-c3nc(-c4ccc(-c5cccc6oc7ccc(-c8cccc9ccccc89)cc7c56)c5ccccc45)nc(-c4cc5ccccc5c5ccccc45)n3)ccc2c1 ; c1ccc2cc(-c3nc(-c4ccc5ccc(-c6cccc7oc8ccc(-c9ccc%10c(ccc%11ccccc%11%10)c9)cc8c67)cc5c4)nc(-c4cc5ccccc5c5ccccc45)n3)ccc2c1 ; c1ccc(-c2ccc3c(c2)oc2cccc(-c4cccc5c(-c6nc(-c7ccc8ccc9ccccc9c8c7)nc(-c7cc8ccccc8c8ccccc78)n6)cccc45)c23)cc1]

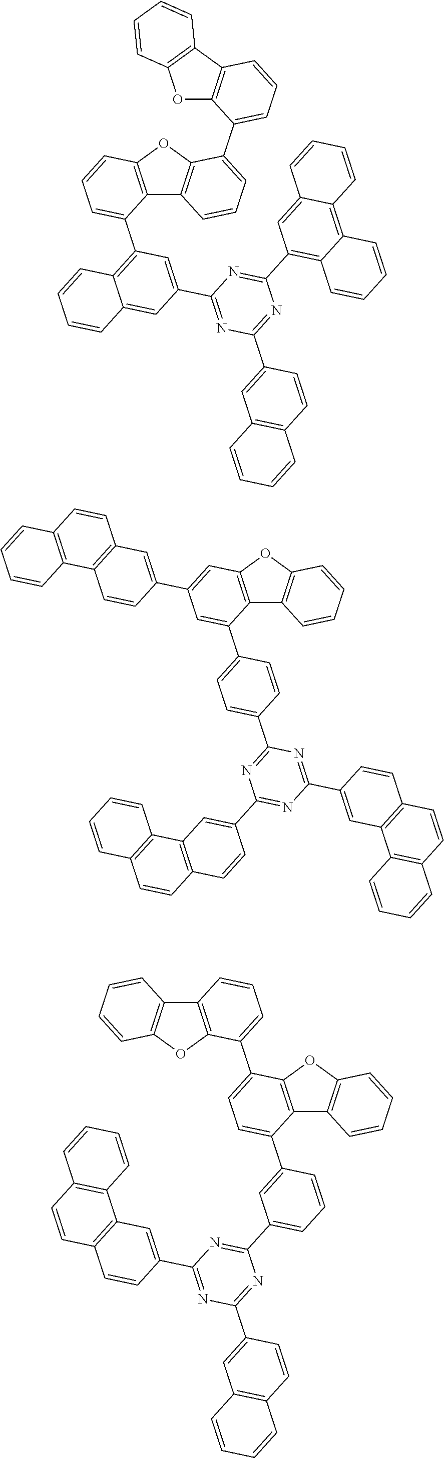 [SMILES: c1ccc2cc(-c3nc(-c4cc(-c5cccc6oc7c(-c8cccc9c8oc8ccccc89)cccc7c56)c5ccccc5c4)nc(-c4cc5ccccc5c5ccccc45)n3)ccc2c1 ; c1ccc2c(c1)ccc1cc(-c3cc(-c4ccc(-c5nc(-c6ccc7ccc8ccccc8c7c6)nc(-c6ccc7ccc8ccccc8c7c6)n5)cc4)c4c(c3)oc3ccccc34)ccc12 ; c1cc(-c2nc(-c3ccc4ccccc4c3)nc(-c3ccc4ccc5ccccc5c4c3)n2)cc(-c2ccc(-c3cccc4c3oc3ccccc34)c3oc4ccccc4c23)c1]

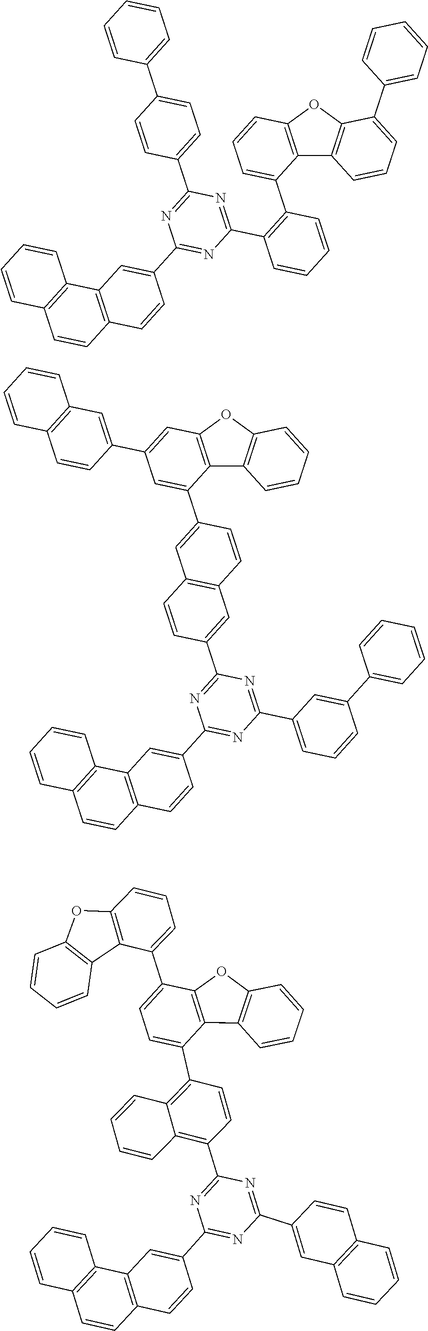

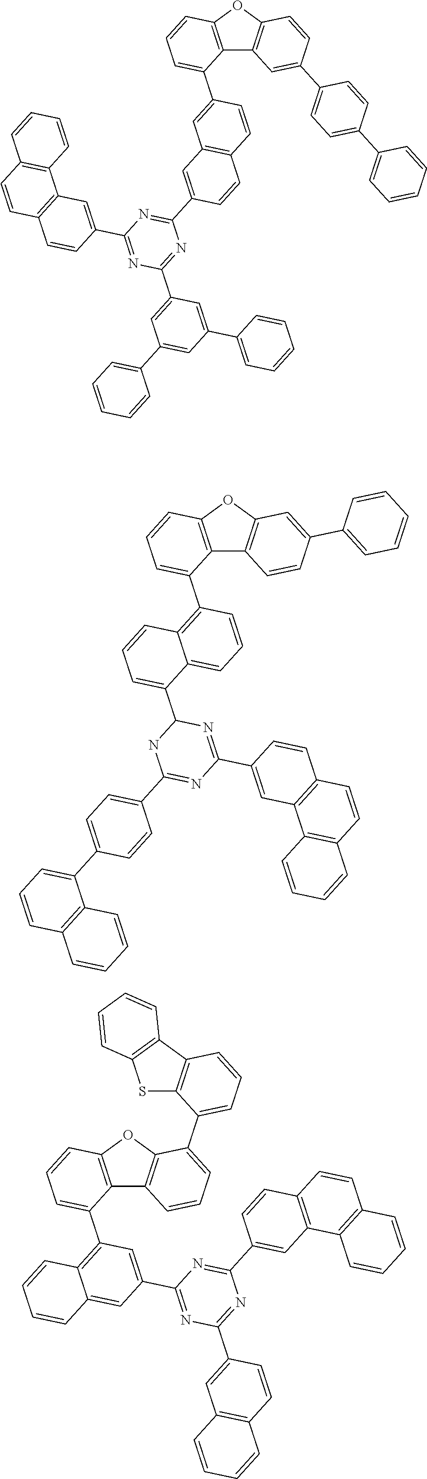

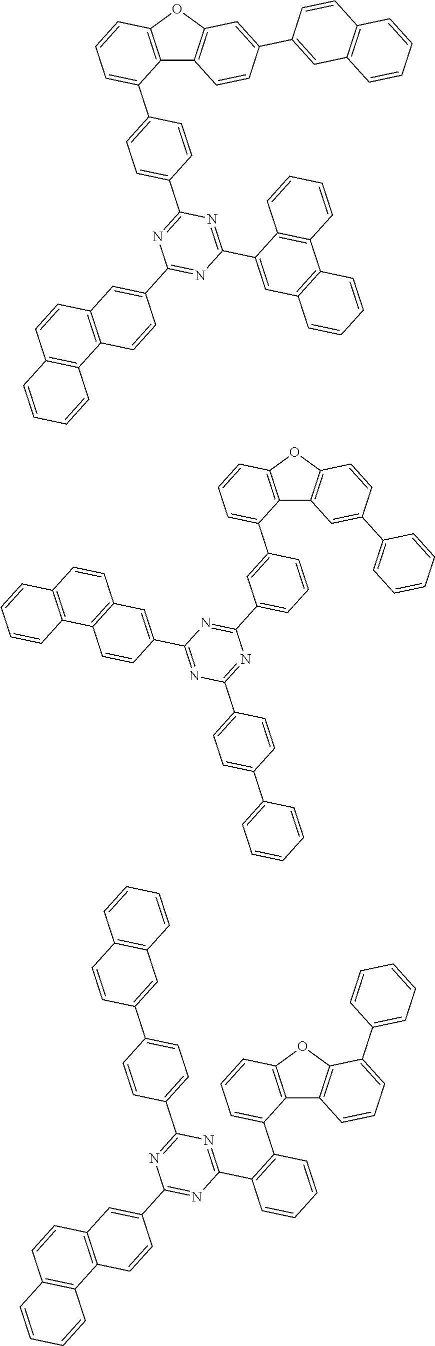 [SMILES: c1ccc2cc(-c3ccc4c(c3)oc3cccc(-c5ccc(-c6nc(-c7ccc8c(ccc9ccccc98)c7)nc(-c7cc8ccccc8c8ccccc78)n6)cc5)c34)ccc2c1 ; c1ccc(-c2ccc(-c3nc(-c4cccc(-c5cccc6oc7ccc(-c8ccccc8)cc7c56)c4)nc(-c4ccc5c(ccc6ccccc65)c4)n3)cc2)cc1 ; c1ccc(-c2cccc3c2oc2cccc(-c4ccccc4-c4nc(-c5ccc(-c6ccc7ccccc7c6)cc5)nc(-c5ccc6c(ccc7ccccc76)c5)n4)c23)cc1]

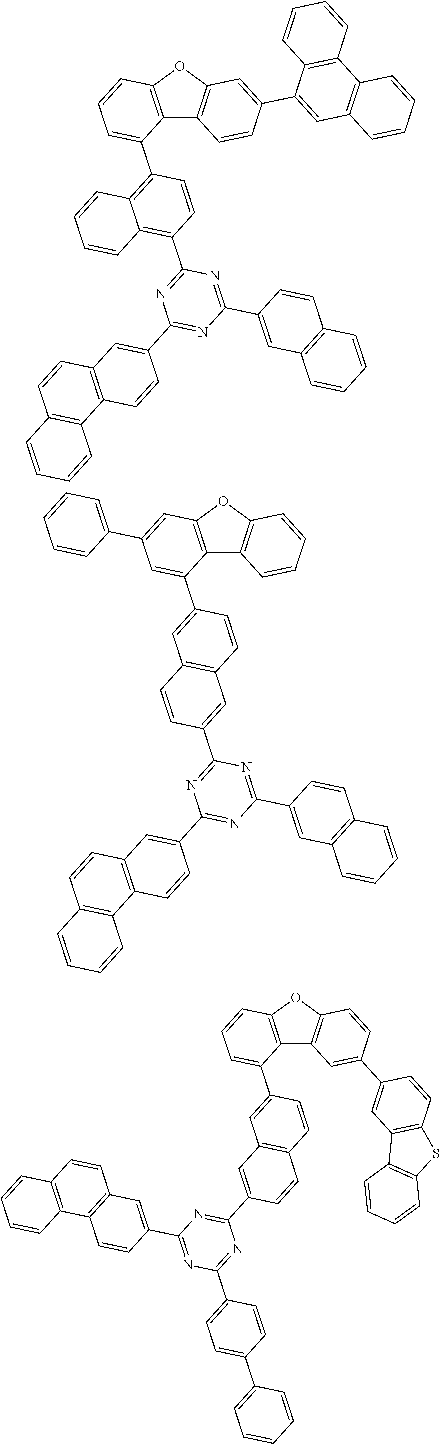

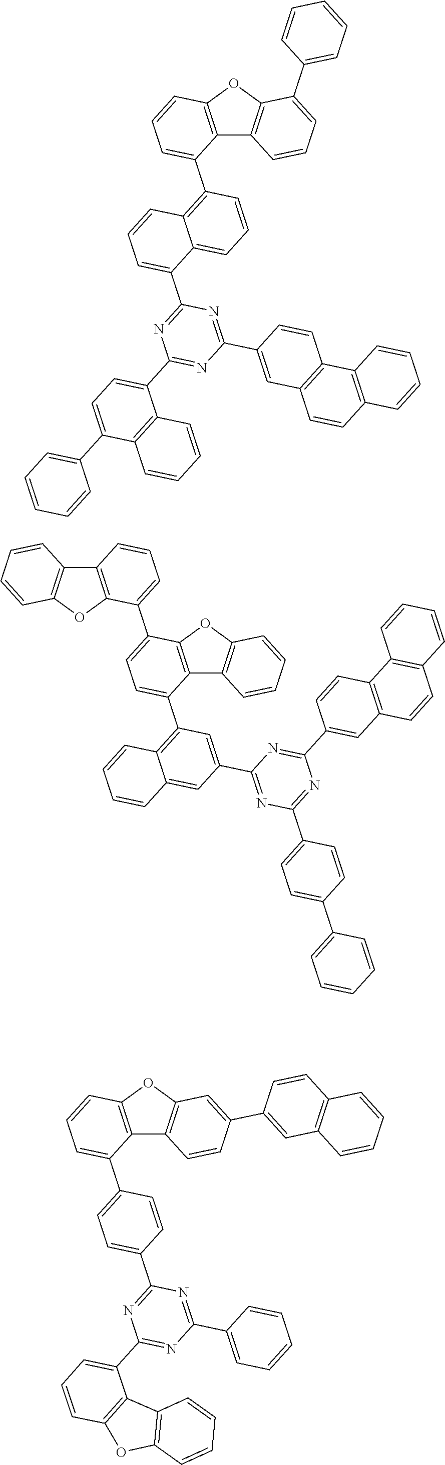

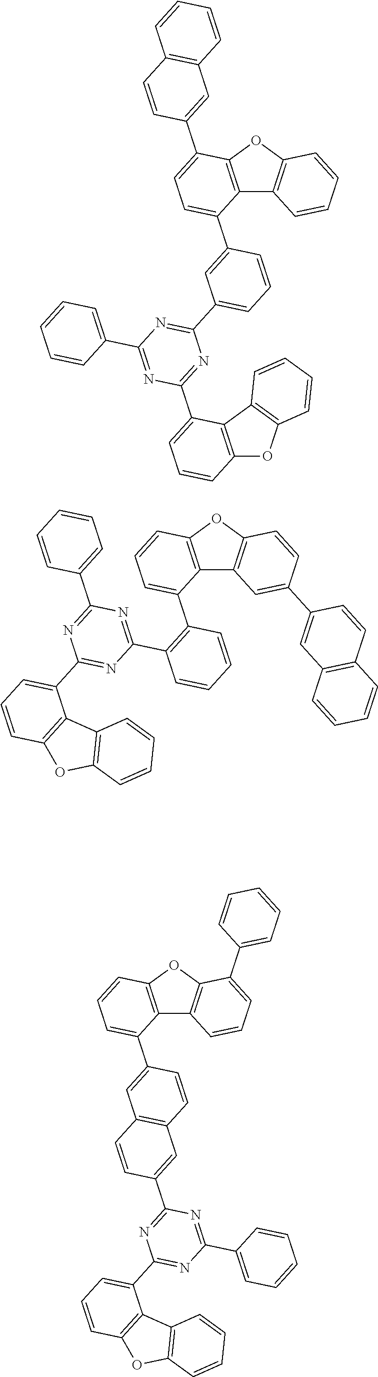

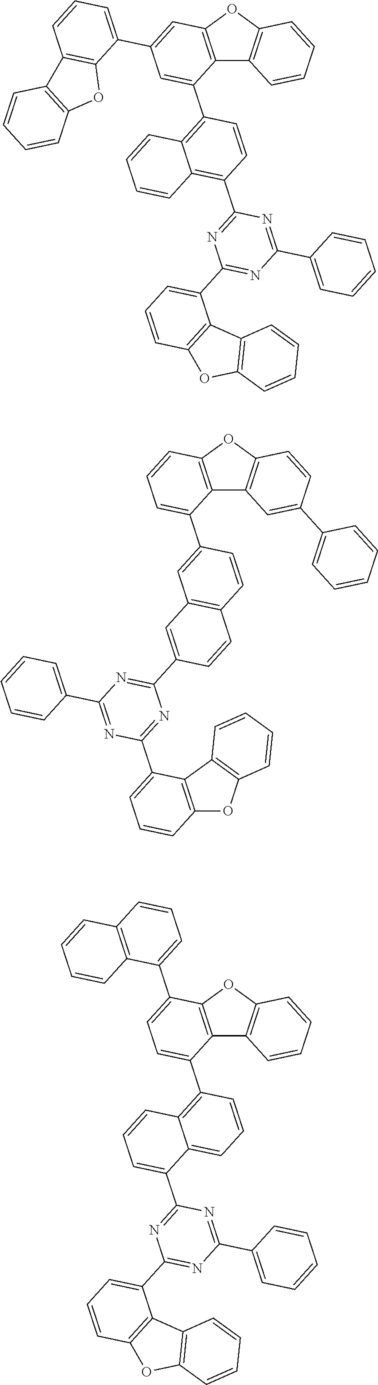 [SMILES: c1ccc(-c2nc(-c3ccc(-c4cc(-c5cccc6c5oc5ccccc56)cc5oc6ccccc6c45)c4ccccc34)nc(-c3cccc4oc5ccccc5c34)n2)cc1 ; c1ccc(-c2ccc3oc4cccc(-c5ccc6ccc(-c7nc(-c8ccccc8)nc(-c8cccc9oc%10ccccc%10c89)n7)cc6c5)c4c3c2)cc1 ; c1ccc(-c2nc(-c3cccc4c(-c5ccc(-c6cccc7ccccc67)c6oc7ccccc7c56)cccc34)nc(-c3cccc4oc5ccccc5c34)n2)cc1]

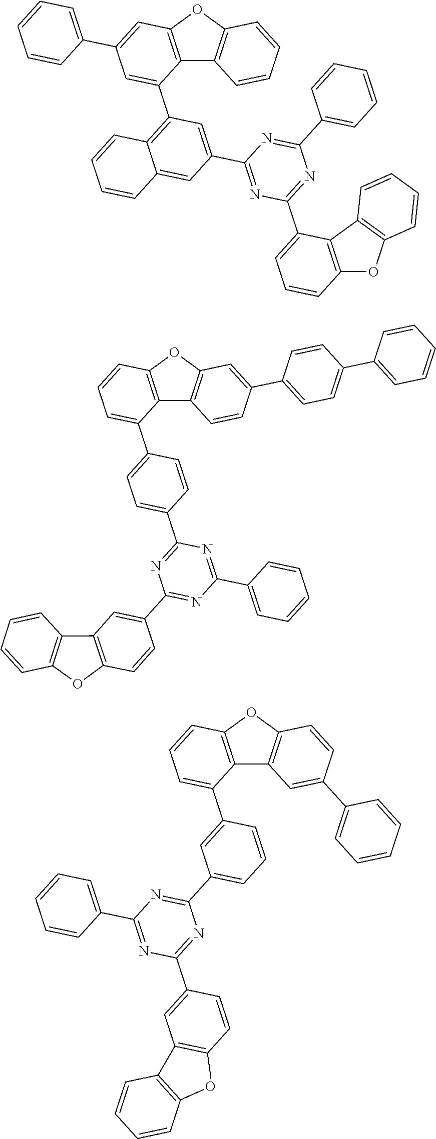 [SMILES: c1ccc(-c2cc(-c3cc(-c4nc(-c5ccccc5)nc(-c5cccc6oc7ccccc7c56)n4)cc4ccccc34)c3c(c2)oc2ccccc23)cc1 ; c1ccc(-c2ccc(-c3ccc4c(c3)oc3cccc(-c5ccc(-c6nc(-c7ccccc7)nc(-c7ccc8oc9ccccc9c8c7)n6)cc5)c34)cc2)cc1 ; c1ccc(-c2ccc3oc4cccc(-c5cccc(-c6nc(-c7ccccc7)nc(-c7ccc8oc9ccccc9c8c7)n6)c5)c4c3c2)cc1]

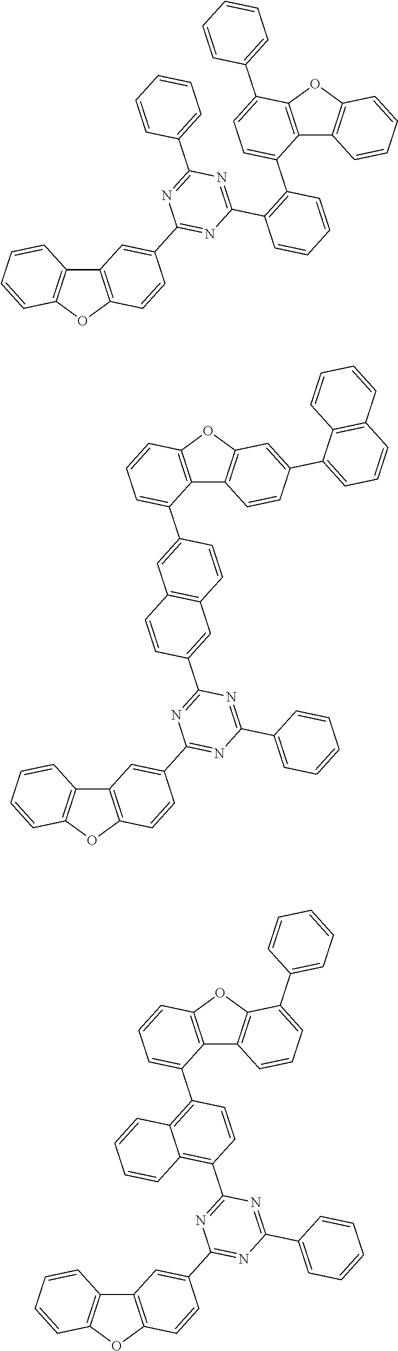 [SMILES: c1ccc(-c2nc(-c3ccc4oc5ccccc5c4c3)nc(-c3ccccc3-c3ccc(-c4ccccc4)c4oc5ccccc5c34)n2)cc1 ; c1ccc(-c2nc(-c3ccc4cc(-c5cccc6oc7cc(-c8cccc9ccccc89)ccc7c56)ccc4c3)nc(-c3ccc4oc5ccccc5c4c3)n2)cc1 ; c1ccc(-c2nc(-c3ccc4oc5ccccc5c4c3)nc(-c3ccc(-c4cccc5oc6c(-c7ccccc7)cccc6c45)c4ccccc34)n2)cc1]

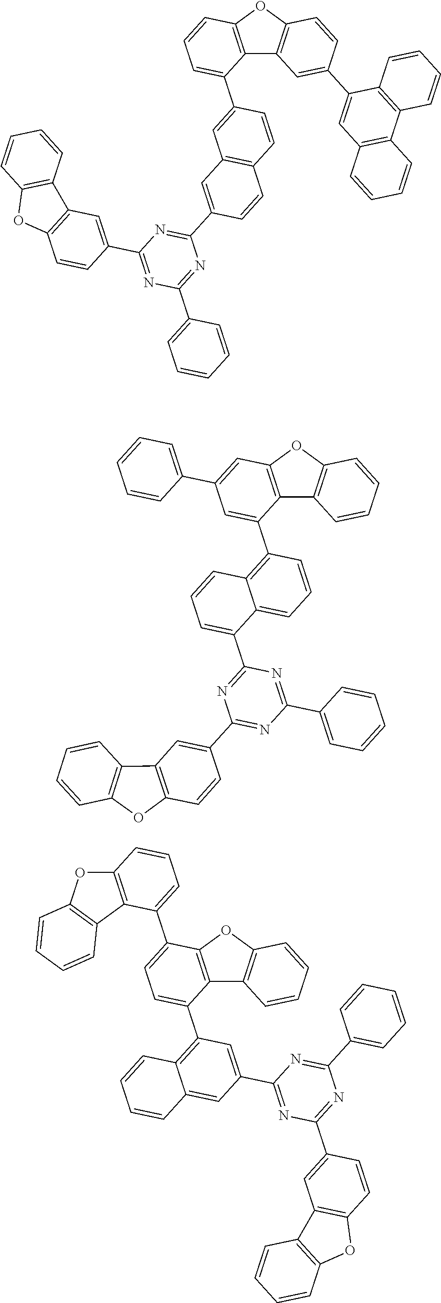

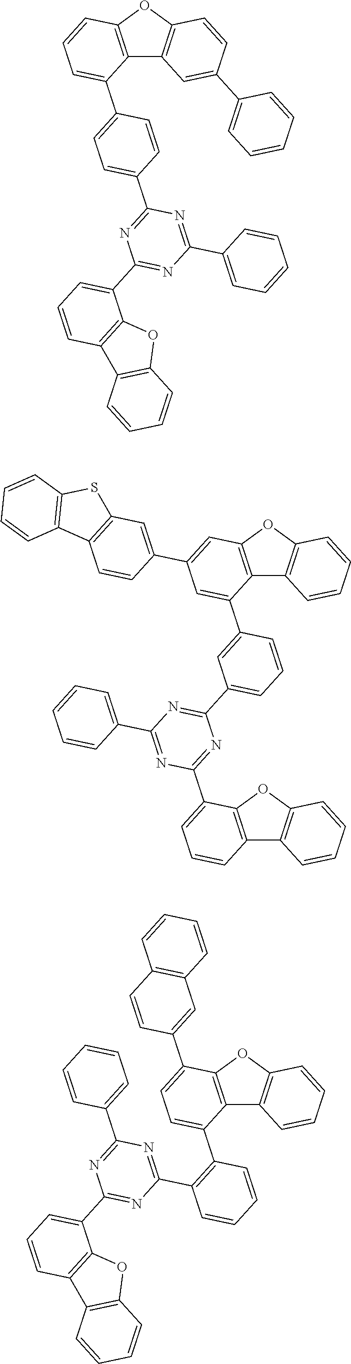 [SMILES: c1ccc(-c2ccc3oc4cccc(-c5ccc(-c6nc(-c7ccccc7)nc(-c7cccc8c7oc7ccccc78)n6)cc5)c4c3c2)cc1 ; c1ccc(-c2nc(-c3cccc(-c4cc(-c5ccc6c(c5)sc5ccccc56)cc5oc6ccccc6c45)c3)nc(-c3cccc4c3oc3ccccc34)n2)cc1 ; c1ccc(-c2nc(-c3ccccc3-c3ccc(-c4ccc5ccccc5c4)c4oc5ccccc5c34)nc(-c3cccc4c3oc3ccccc34)n2)cc1]

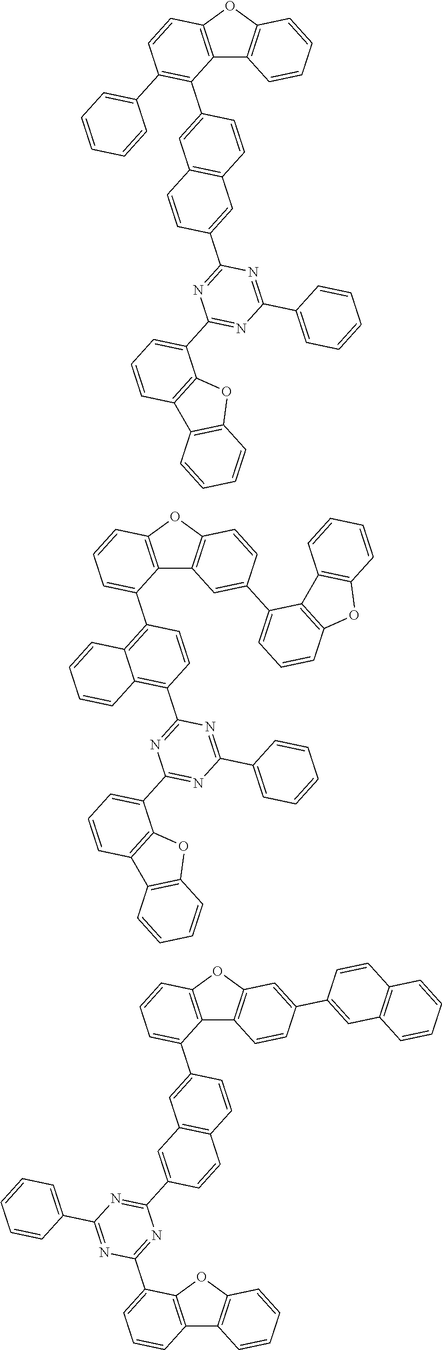

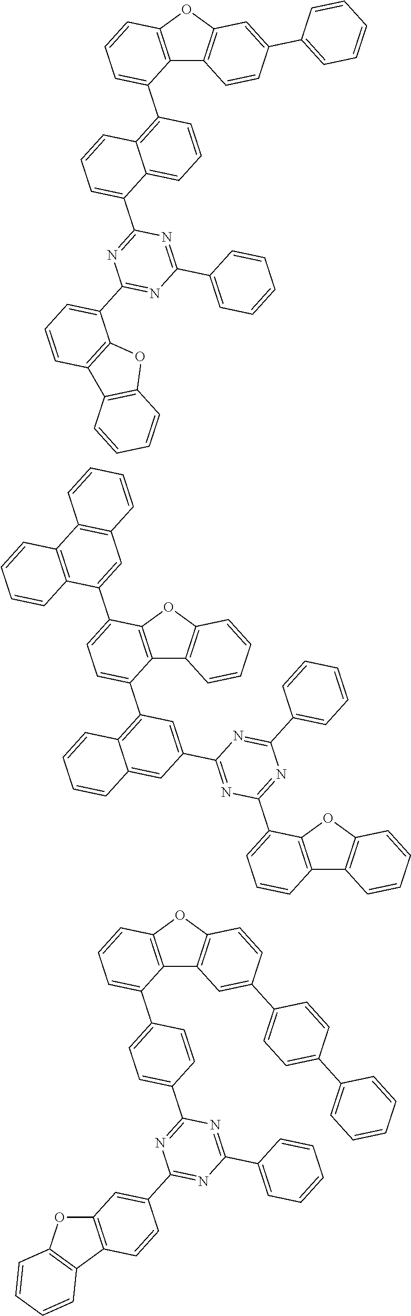

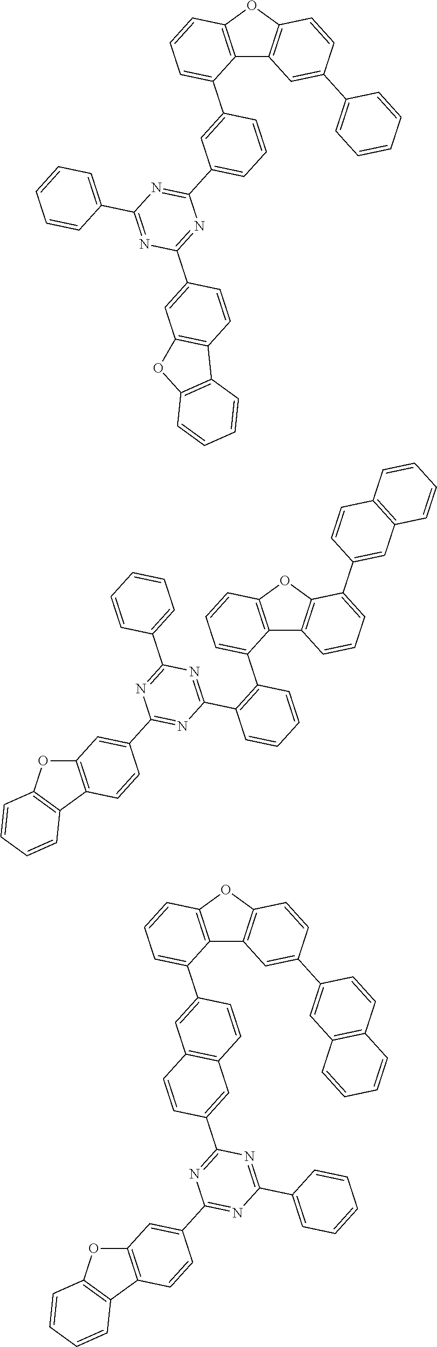 [SMILES: c1ccc(-c2ccc3oc4cccc(-c5cccc(-c6nc(-c7ccccc7)nc(-c7ccc8c(c7)oc7ccccc78)n6)c5)c4c3c2)cc1 ; c1ccc(-c2nc(-c3ccc4c(c3)oc3ccccc34)nc(-c3ccccc3-c3cccc4oc5c(-c6ccc7ccccc7c6)cccc5c34)n2)cc1 ; c1ccc(-c2nc(-c3ccc4cc(-c5cccc6oc7ccc(-c8ccc9ccccc9c8)cc7c56)ccc4c3)nc(-c3ccc4c(c3)oc3ccccc34)n2)cc1]

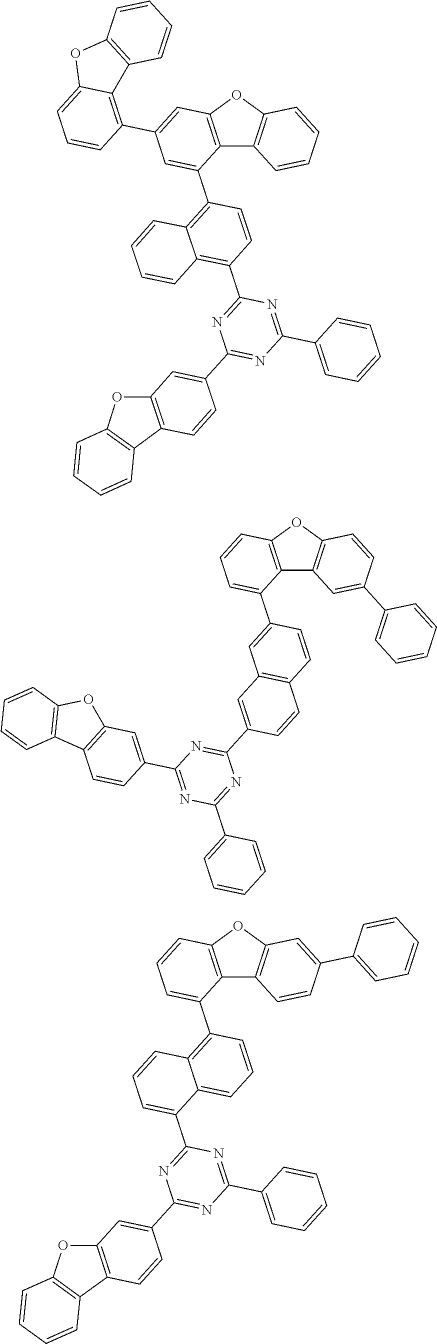

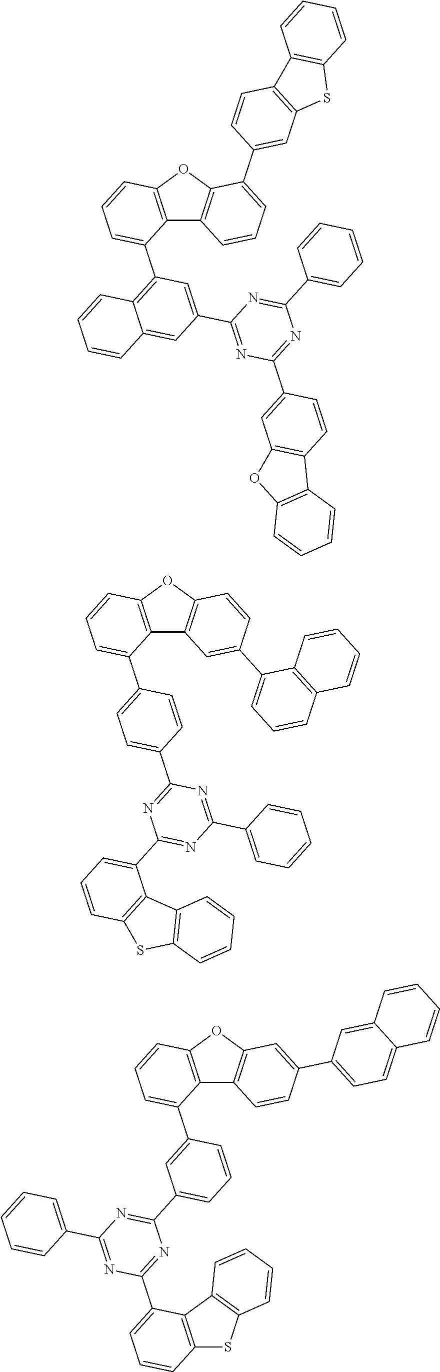 [SMILES: c1ccc(-c2nc(-c3cc(-c4cccc5oc6c(-c7ccc8c(c7)sc7ccccc78)cccc6c45)c4ccccc4c3)nc(-c3ccc4c(c3)oc3ccccc34)n2)cc1 ; c1ccc(-c2nc(-c3ccc(-c4cccc5oc6ccc(-c7cccc8ccccc78)cc6c45)cc3)nc(-c3cccc4sc5ccccc5c34)n2)cc1 ; c1ccc(-c2nc(-c3cccc(-c4cccc5oc6cc(-c7ccc8ccccc8c7)ccc6c45)c3)nc(-c3cccc4sc5ccccc5c34)n2)cc1]

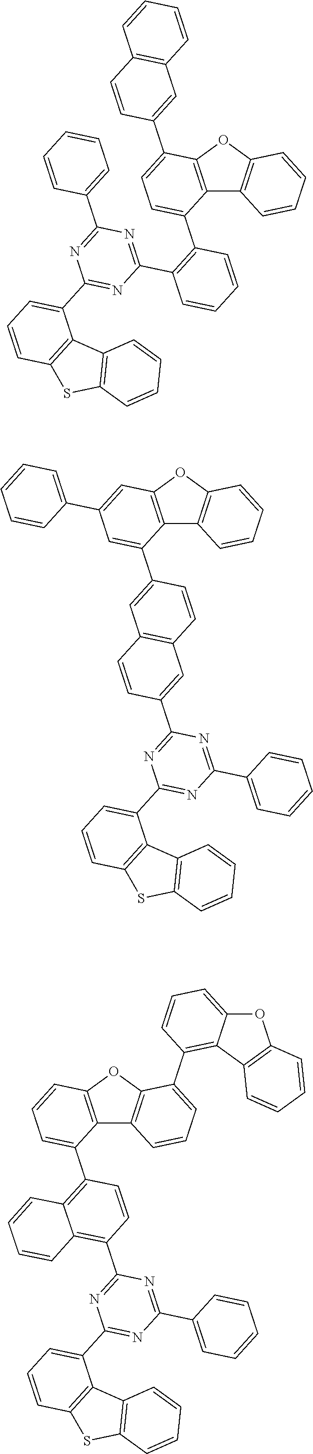 [SMILES: c1ccc(-c2nc(-c3ccccc3-c3ccc(-c4ccc5ccccc5c4)c4oc5ccccc5c34)nc(-c3cccc4sc5ccccc5c34)n2)cc1 ; c1ccc(-c2cc(-c3ccc4cc(-c5nc(-c6ccccc6)nc(-c6cccc7sc8ccccc8c67)n5)ccc4c3)c3c(c2)oc2ccccc23)cc1 ; c1ccc(-c2nc(-c3ccc(-c4cccc5oc6c(-c7cccc8oc9ccccc9c78)cccc6c45)c4ccccc34)nc(-c3cccc4sc5ccccc5c34)n2)cc1]

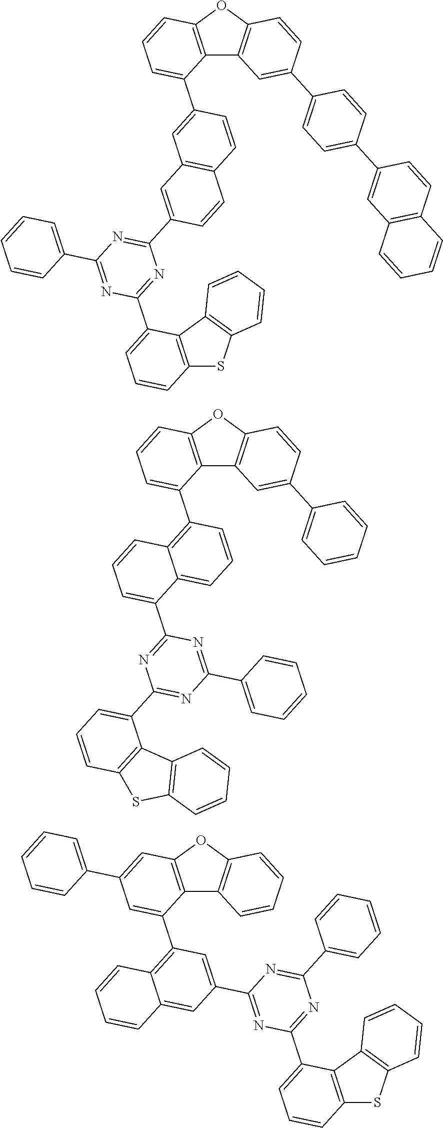

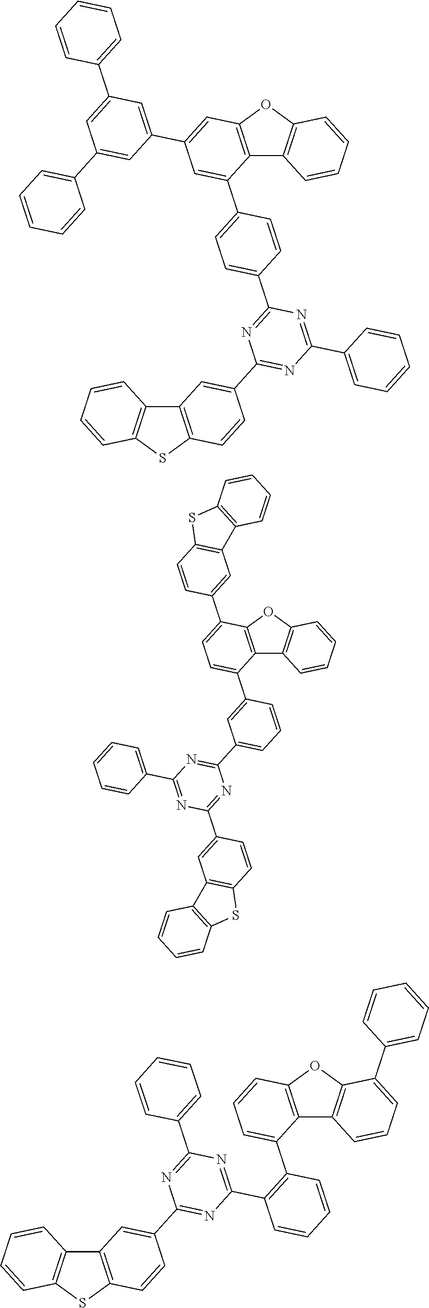

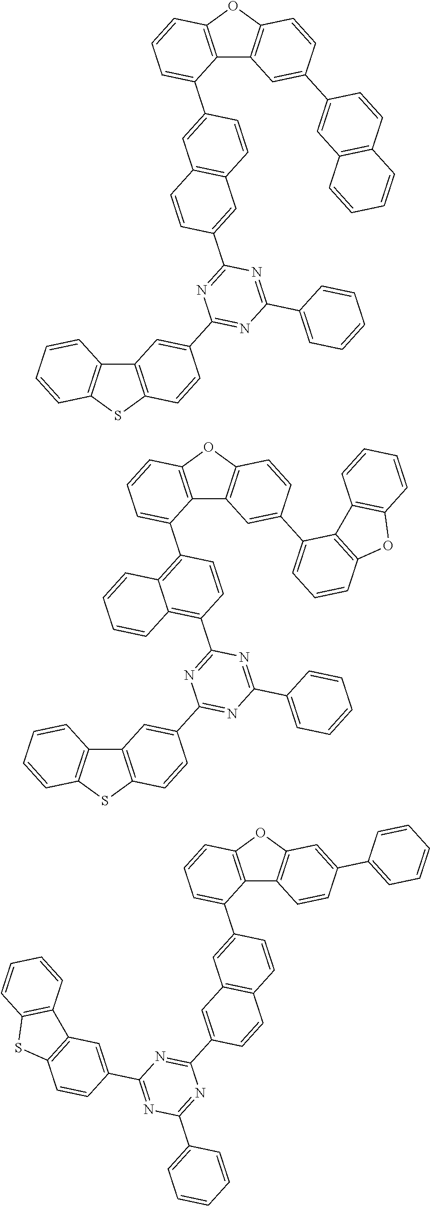 [SMILES: c1ccc(-c2nc(-c3ccc4cc(-c5cccc6oc7ccc(-c8ccc9ccccc9c8)cc7c56)ccc4c3)nc(-c3ccc4sc5ccccc5c4c3)n2)cc1 ; c1ccc(-c2nc(-c3ccc4sc5ccccc5c4c3)nc(-c3ccc(-c4cccc5oc6ccc(-c7cccc8oc9ccccc9c78)cc6c45)c4ccccc34)n2)cc1 ; c1ccc(-c2ccc3c(c2)oc2cccc(-c4ccc5ccc(-c6nc(-c7ccccc7)nc(-c7ccc8sc9ccccc9c8c7)n6)cc5c4)c23)cc1]

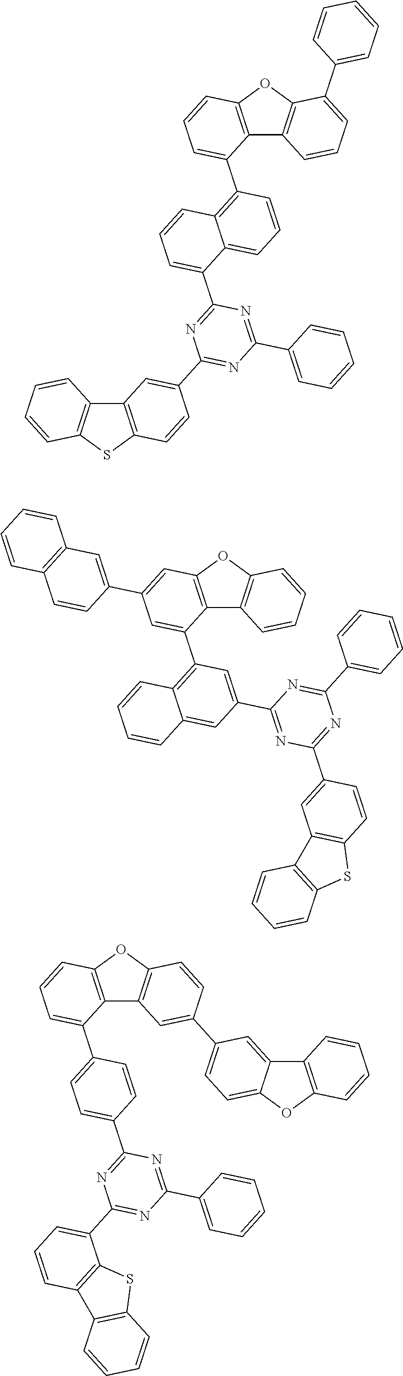 [SMILES: c1ccc(-c2nc(-c3ccc4sc5ccccc5c4c3)nc(-c3cccc4c(-c5cccc6oc7c(-c8ccccc8)cccc7c56)cccc34)n2)cc1 ; c1ccc(-c2nc(-c3cc(-c4cc(-c5ccc6ccccc6c5)cc5oc6ccccc6c45)c4ccccc4c3)nc(-c3ccc4sc5ccccc5c4c3)n2)cc1 ; c1ccc(-c2nc(-c3ccc(-c4cccc5oc6ccc(-c7ccc8oc9ccccc9c8c7)cc6c45)cc3)nc(-c3cccc4c3sc3ccccc34)n2)cc1]

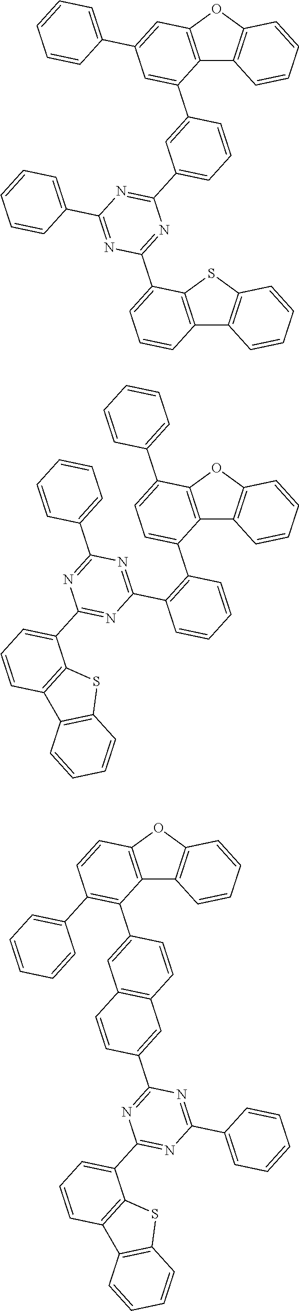

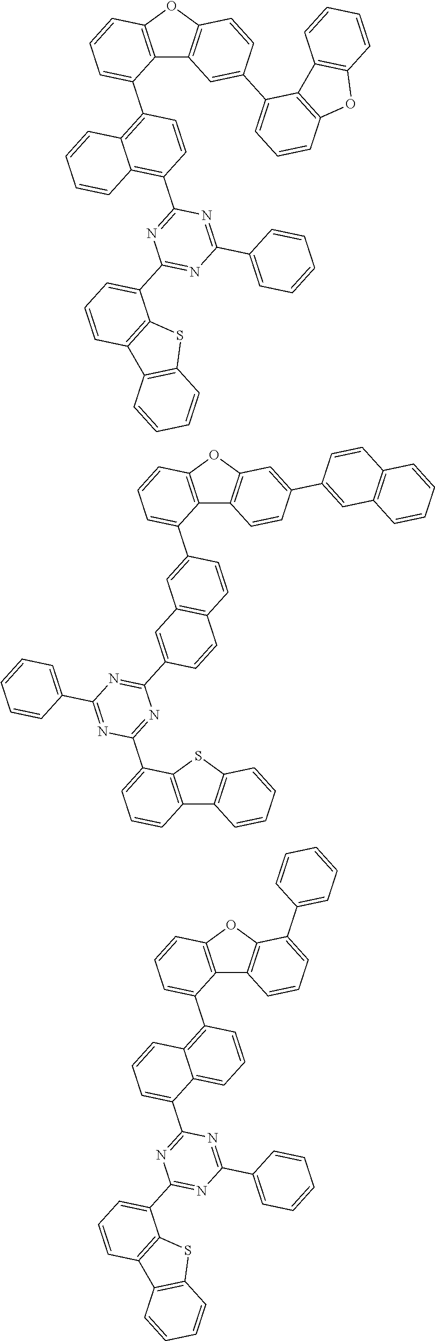

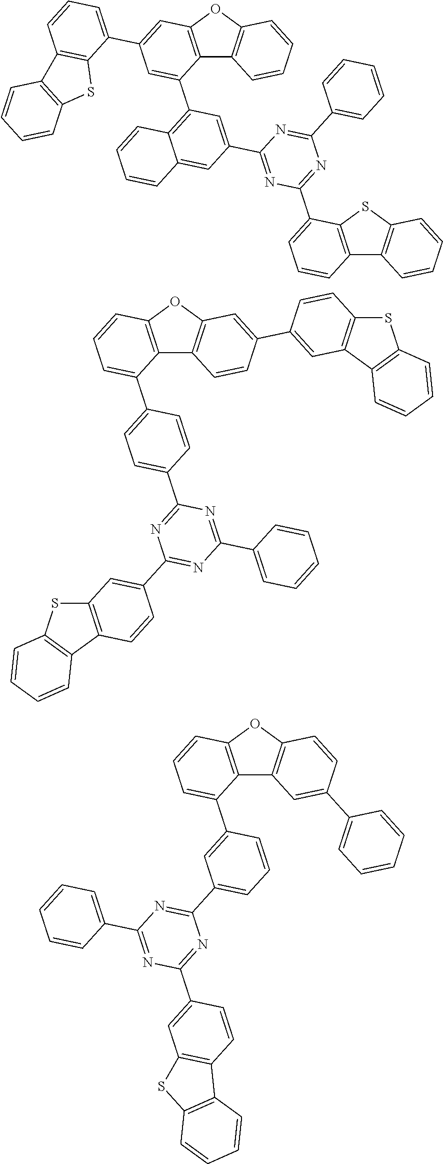

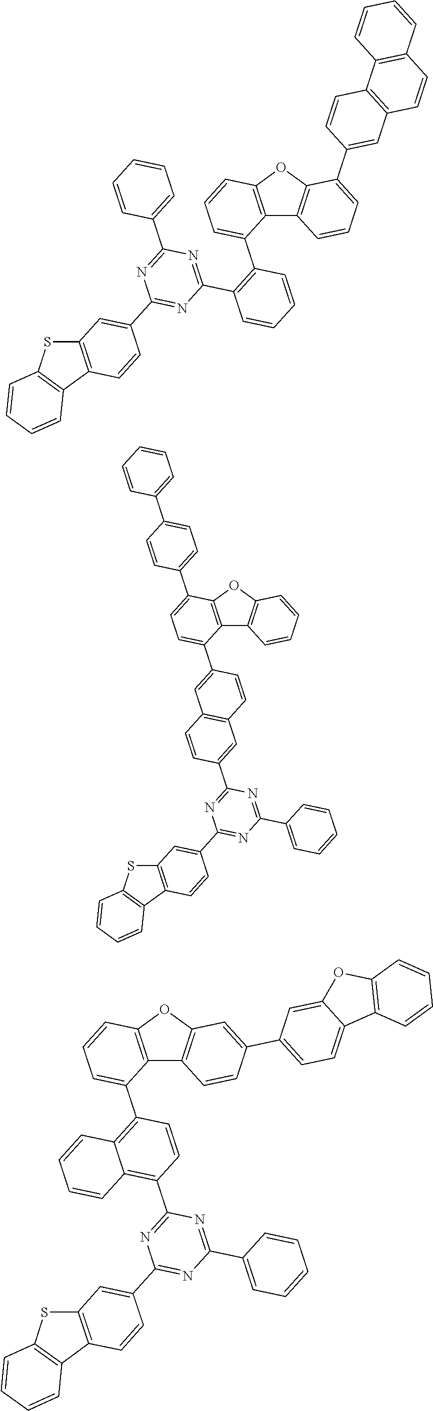 [SMILES: c1ccc(-c2nc(-c3ccc4c(c3)sc3ccccc34)nc(-c3ccccc3-c3cccc4oc5c(-c6ccc7c(ccc8ccccc87)c6)cccc5c34)n2)cc1 ; c1ccc(-c2ccc(-c3ccc(-c4ccc5cc(-c6nc(-c7ccccc7)nc(-c7ccc8c(c7)sc7ccccc78)n6)ccc5c4)c4c3oc3ccccc34)cc2)cc1 ; c1ccc(-c2nc(-c3ccc4c(c3)sc3ccccc34)nc(-c3ccc(-c4cccc5oc6cc(-c7ccc8c(c7)oc7ccccc78)ccc6c45)c4ccccc34)n2)cc1]

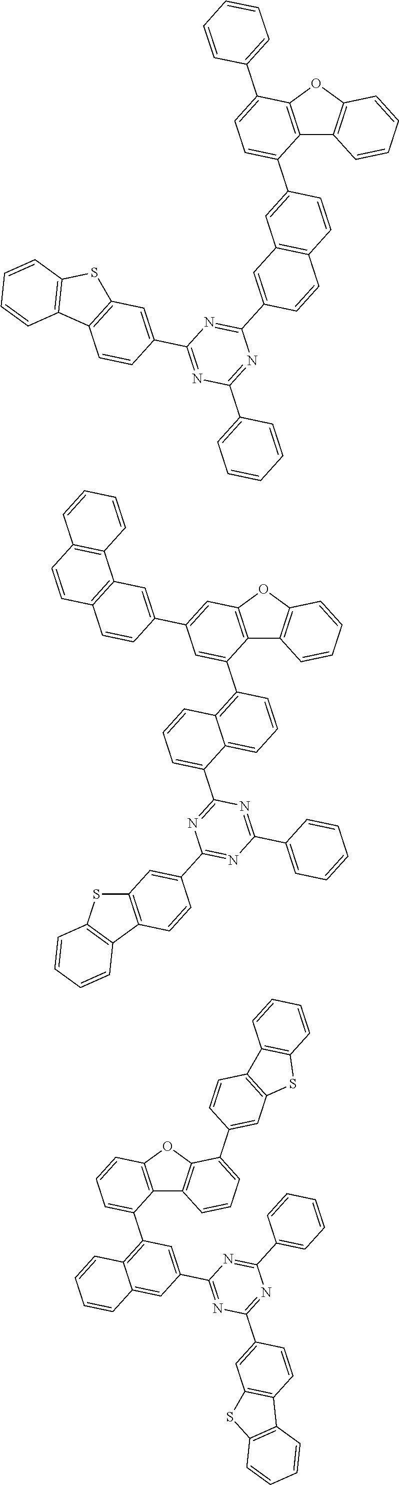 [SMILES: c1ccc(-c2nc(-c3ccc4ccc(-c5ccc(-c6ccccc6)c6oc7ccccc7c56)cc4c3)nc(-c3ccc4c(c3)sc3ccccc34)n2)cc1 ; c1ccc(-c2nc(-c3ccc4c(c3)sc3ccccc34)nc(-c3cccc4c(-c5cc(-c6ccc7ccc8ccccc8c7c6)cc6oc7ccccc7c56)cccc34)n2)cc1 ; c1ccc(-c2nc(-c3cc(-c4cccc5oc6c(-c7ccc8c(c7)sc7ccccc78)cccc6c45)c4ccccc4c3)nc(-c3ccc4c(c3)sc3ccccc34)n2)cc1]

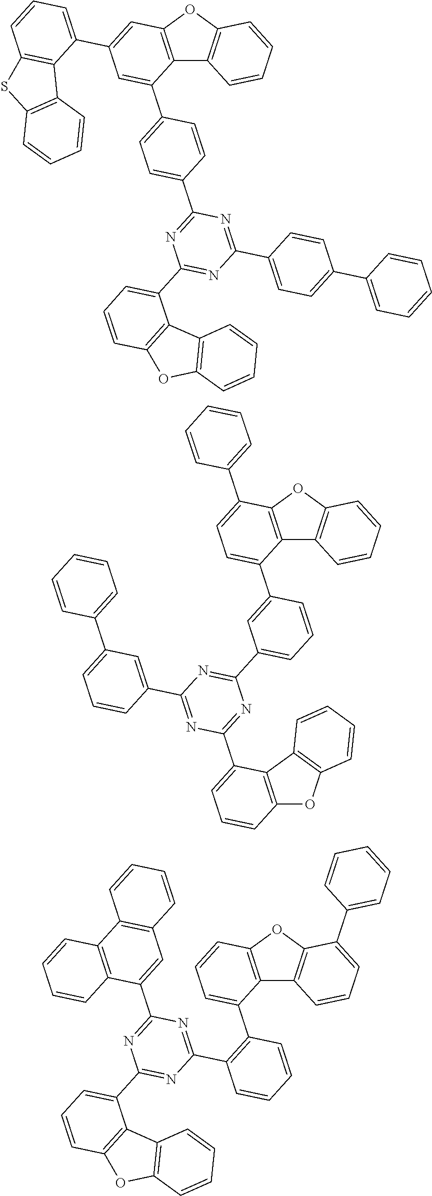

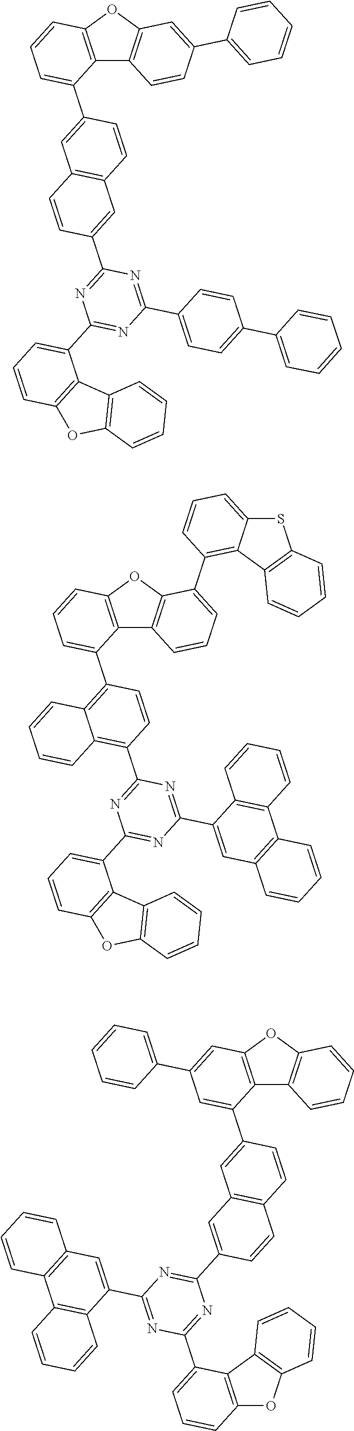 [SMILES: c1ccc(-c2ccc(-c3nc(-c4ccc5cc(-c6cccc7oc8cc(-c9ccccc9)ccc8c67)ccc5c4)nc(-c4cccc5oc6ccccc6c45)n3)cc2)cc1 ; c1ccc2c(c1)cc(-c1nc(-c3ccc(-c4cccc5oc6c(-c7cccc8sc9ccccc9c78)cccc6c45)c4ccccc34)nc(-c3cccc4oc5ccccc5c34)n1)c1ccccc12 ; c1ccc(-c2cc(-c3ccc4ccc(-c5nc(-c6cc7ccccc7c7ccccc67)nc(-c6cccc7oc8ccccc8c67)n5)cc4c3)c3c(c2)oc2ccccc23)cc1]

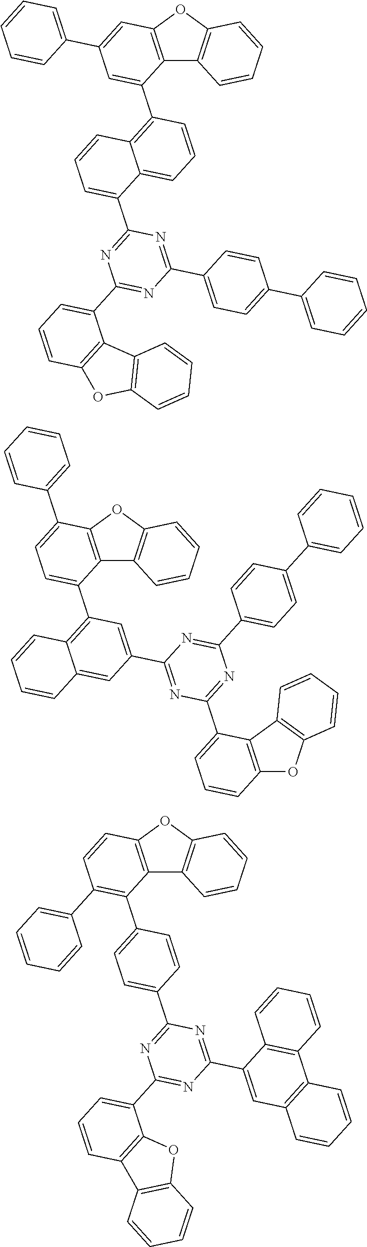

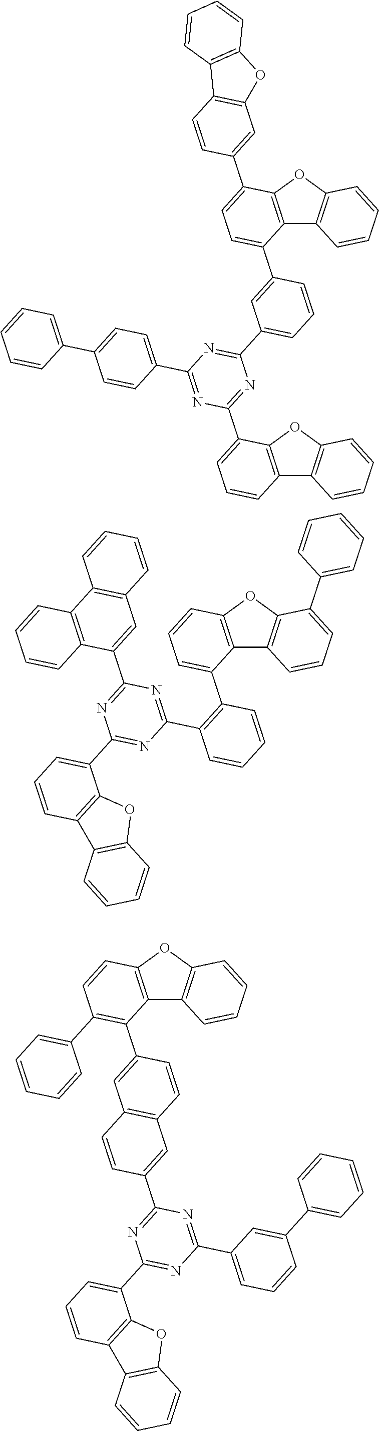

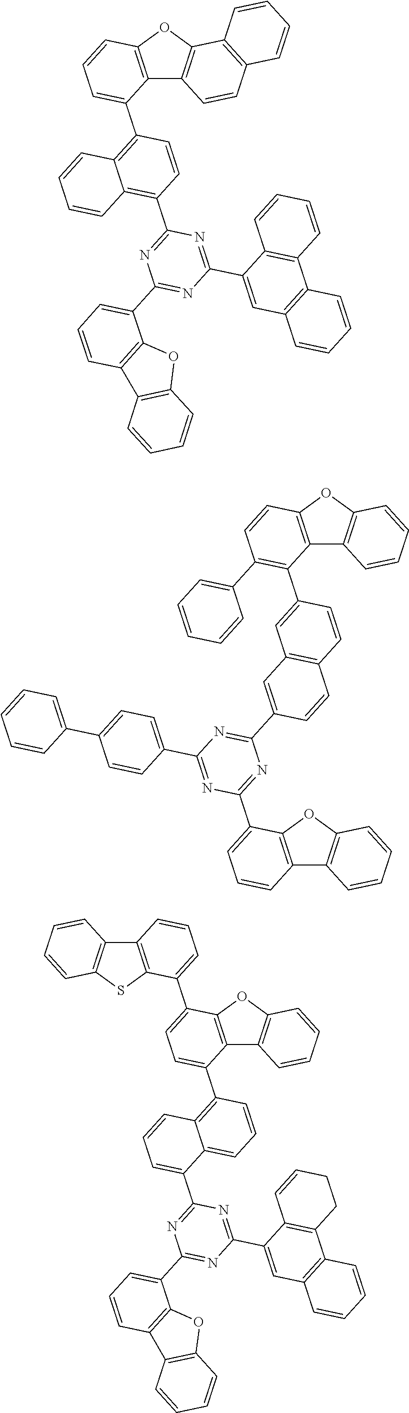 [SMILES: c1ccc2c(c1)cc(-c1nc(-c3ccc(-c4cccc5oc6c7ccccc7ccc6c45)c4ccccc34)nc(-c3cccc4c3oc3ccccc34)n1)c1ccccc12 ; c1ccc(-c2ccc(-c3nc(-c4ccc5ccc(-c6c(-c7ccccc7)ccc7oc8ccccc8c67)cc5c4)nc(-c4cccc5c4oc4ccccc45)n3)cc2)cc1 ; C1=Cc2c(-c3nc(-c4cccc5c(-c6ccc(-c7cccc8c7sc7ccccc78)c7oc8ccccc8c67)cccc45)nc(-c4cccc5c4oc4ccccc45)n3)cc3ccccc3c2CC1]

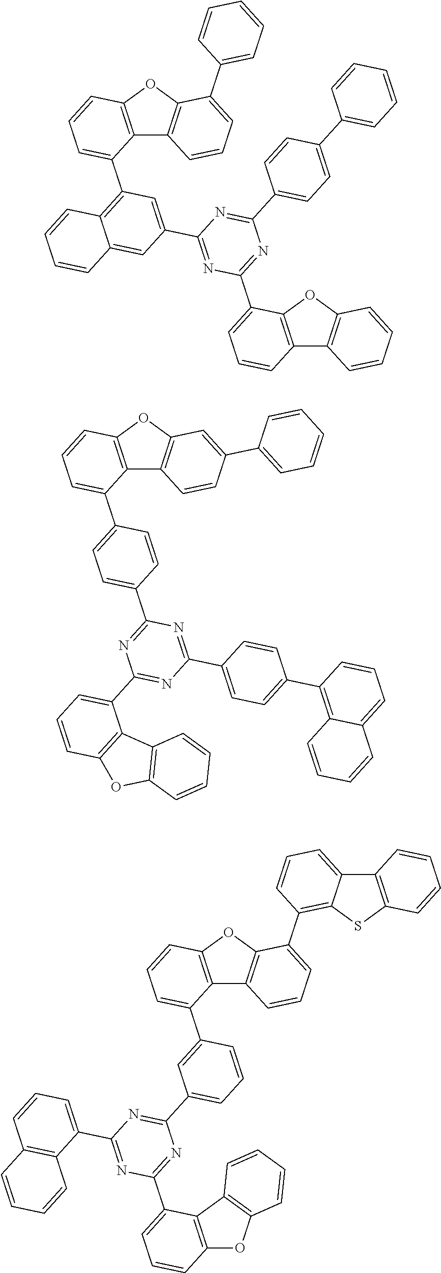

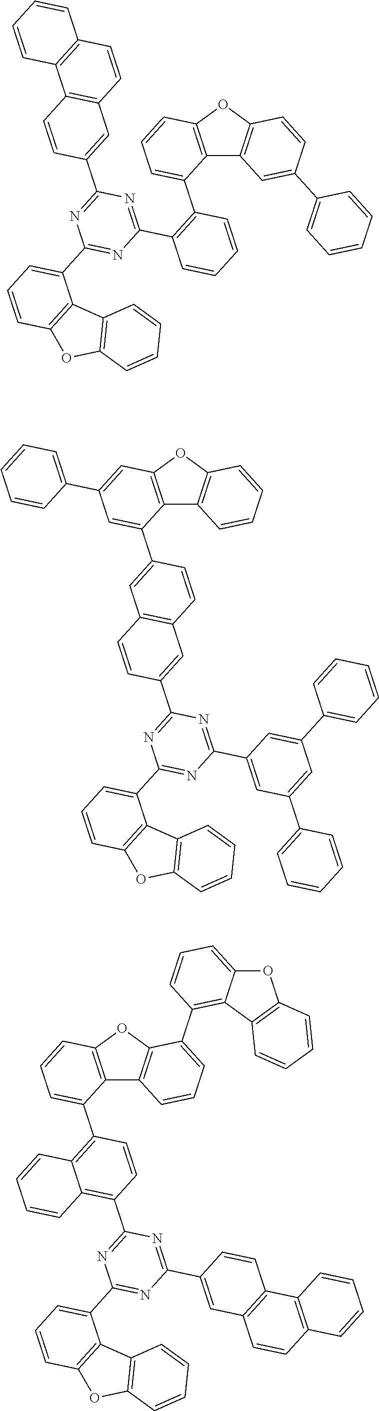 [SMILES: c1ccc(-c2ccc3oc4cccc(-c5ccccc5-c5nc(-c6ccc7c(ccc8ccccc87)c6)nc(-c6cccc7oc8ccccc8c67)n5)c4c3c2)cc1 ; c1ccc(-c2cc(-c3ccccc3)cc(-c3nc(-c4ccc5cc(-c6cc(-c7ccccc7)cc7oc8ccccc8c67)ccc5c4)nc(-c4cccc5oc6ccccc6c45)n3)c2)cc1 ; c1ccc2c(c1)ccc1cc(-c3nc(-c4ccc(-c5cccc6oc7c(-c8cccc9oc%10ccccc%10c89)cccc7c56)c5ccccc45)nc(-c4cccc5oc6ccccc6c45)n3)ccc12]

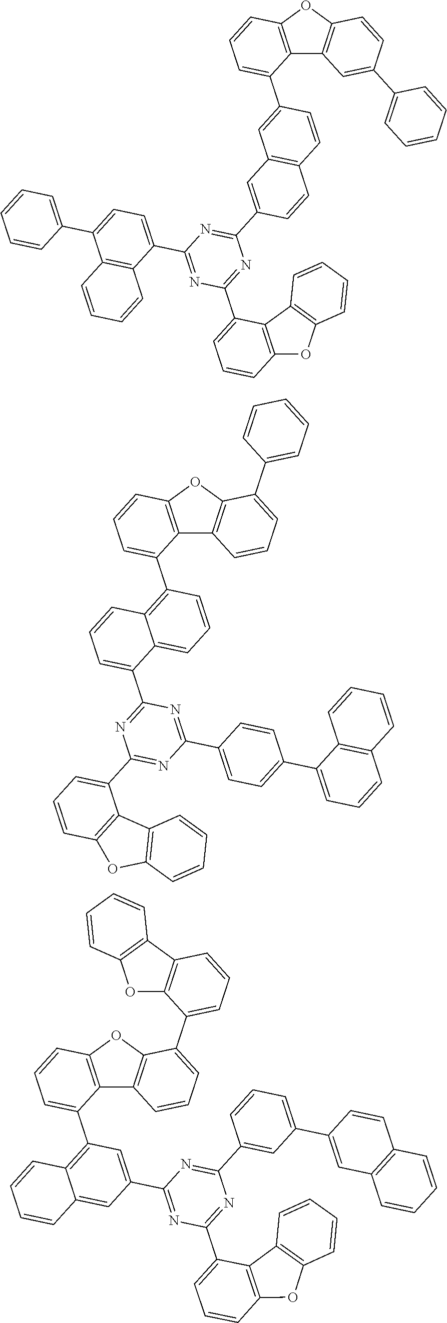

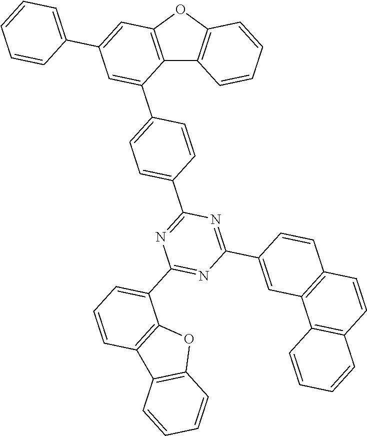 [SMILES: c1ccc(-c2cc(-c3ccc(-c4nc(-c5ccc6ccc7ccccc7c6c5)nc(-c5cccc6c5oc5ccccc56)n4)cc3)c3c(c2)oc2ccccc23)cc1]

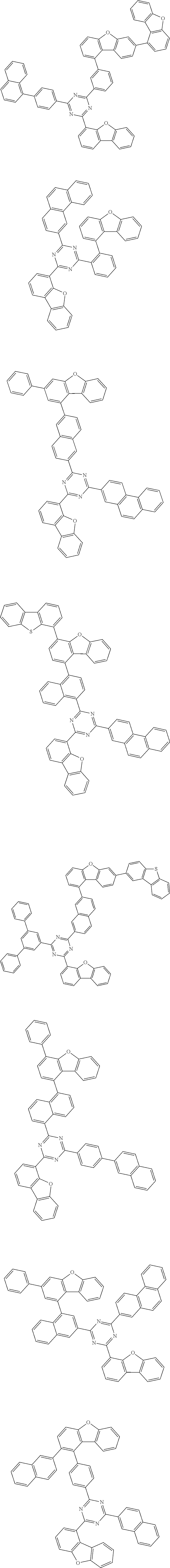

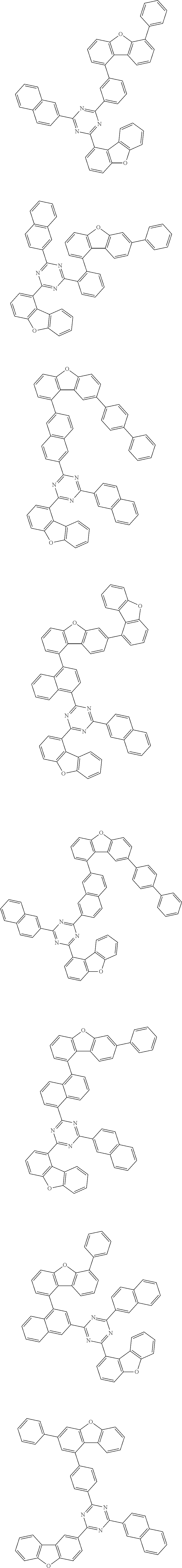

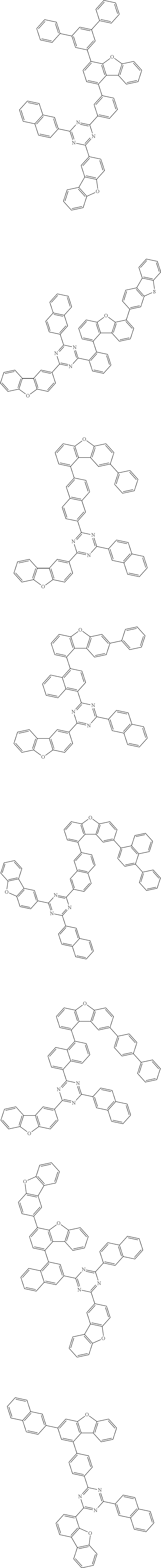

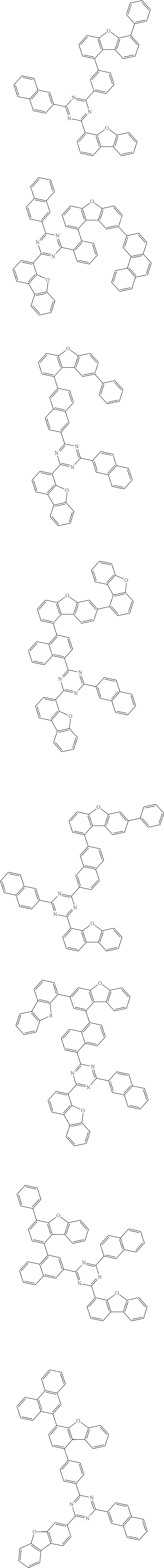

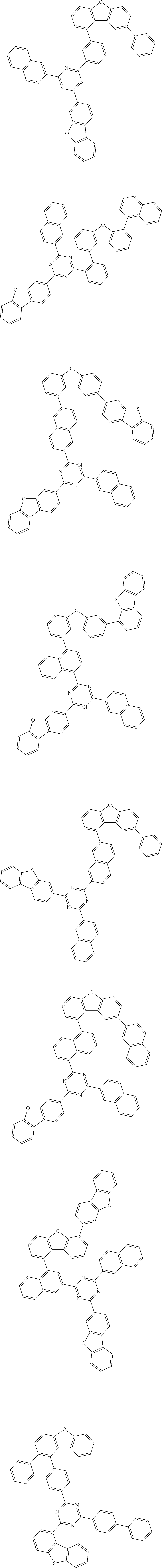

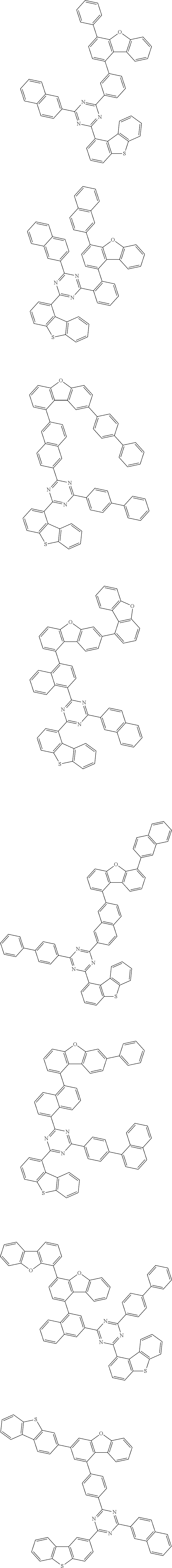

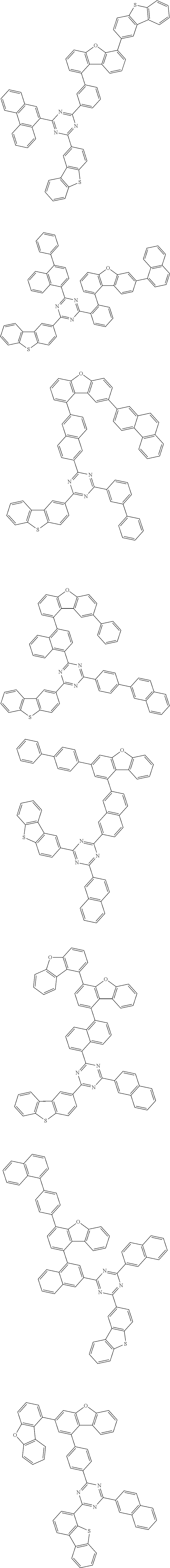

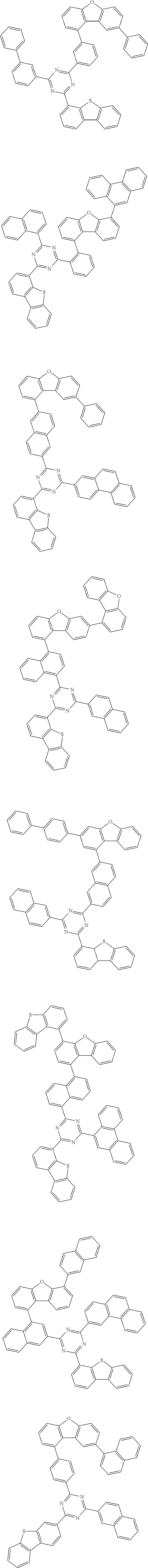

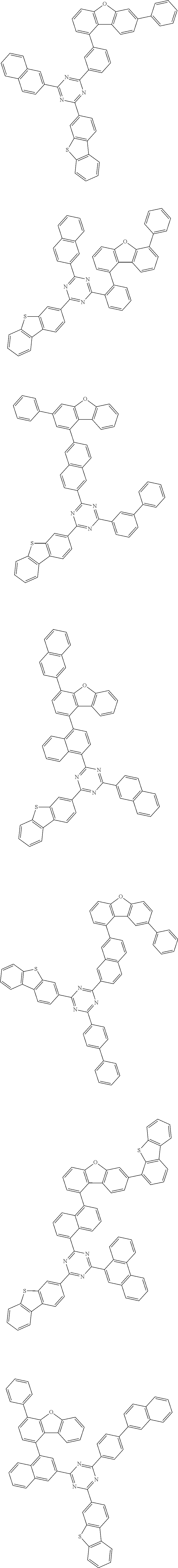

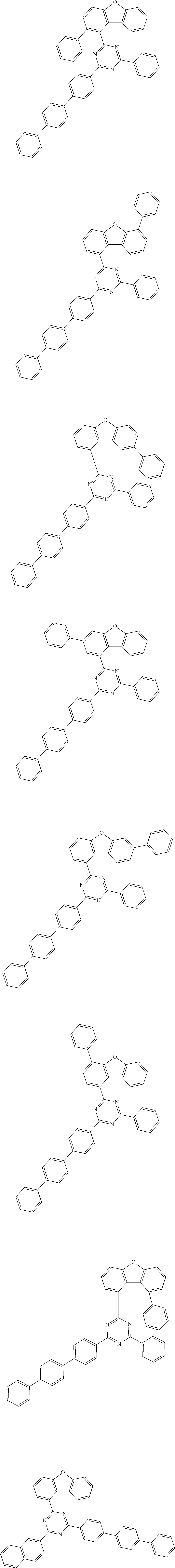

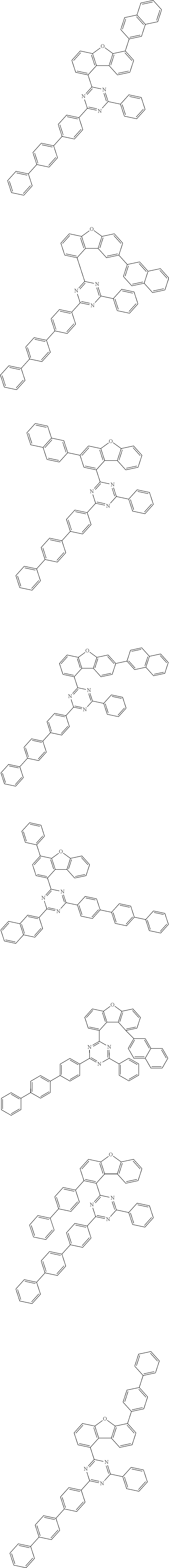

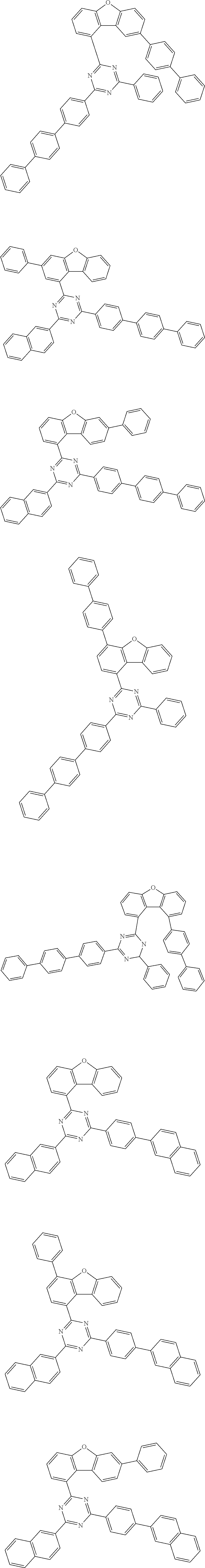

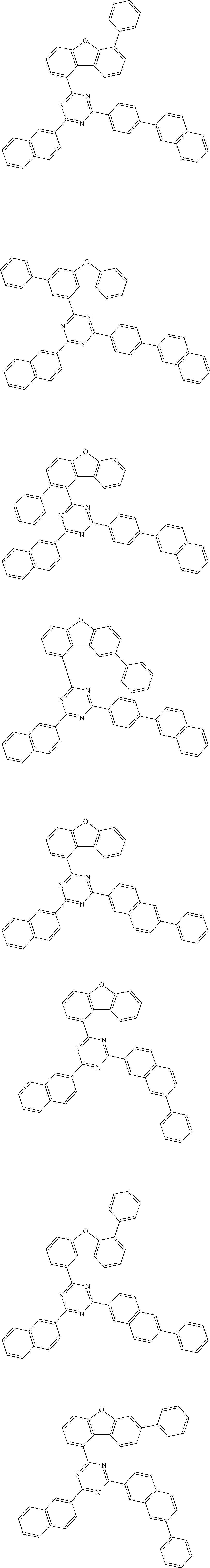

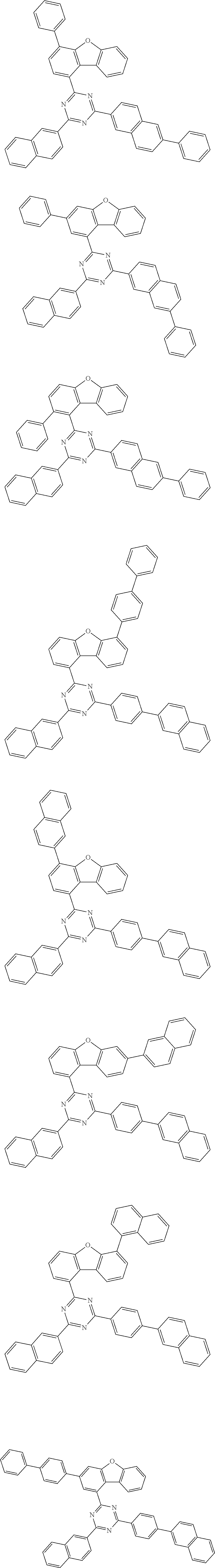

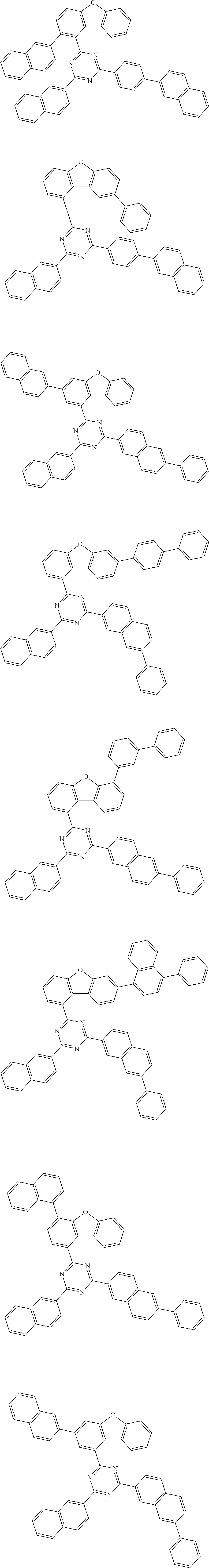

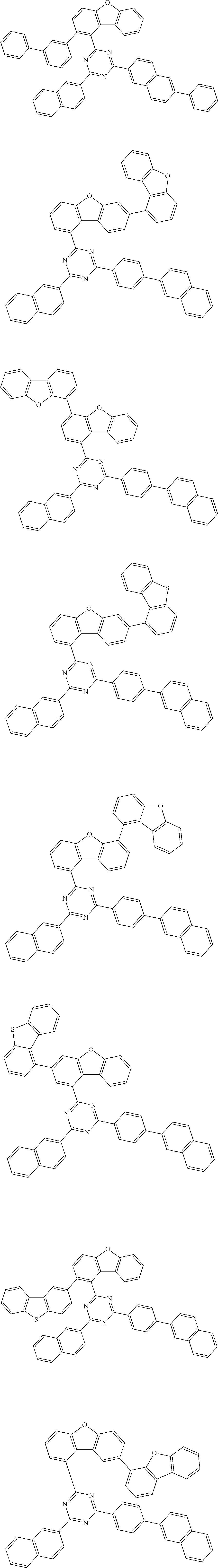

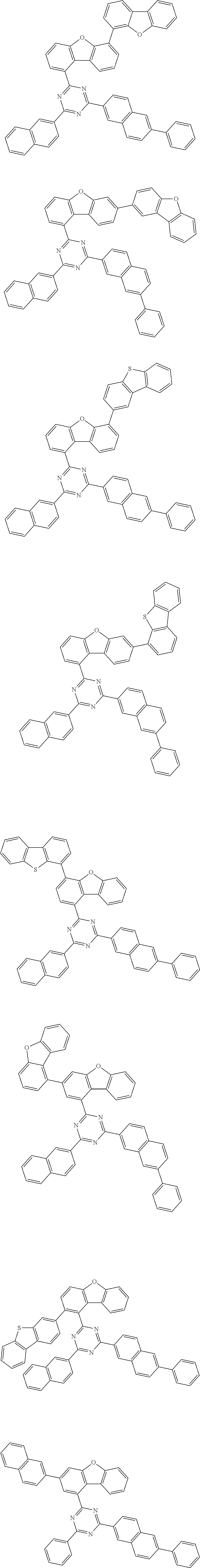

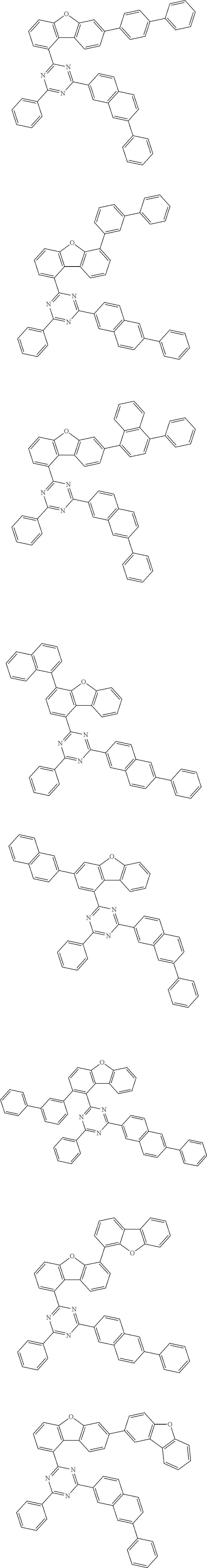

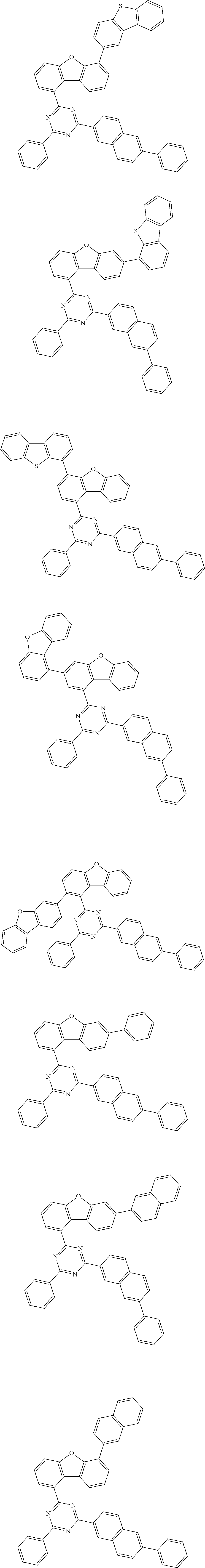

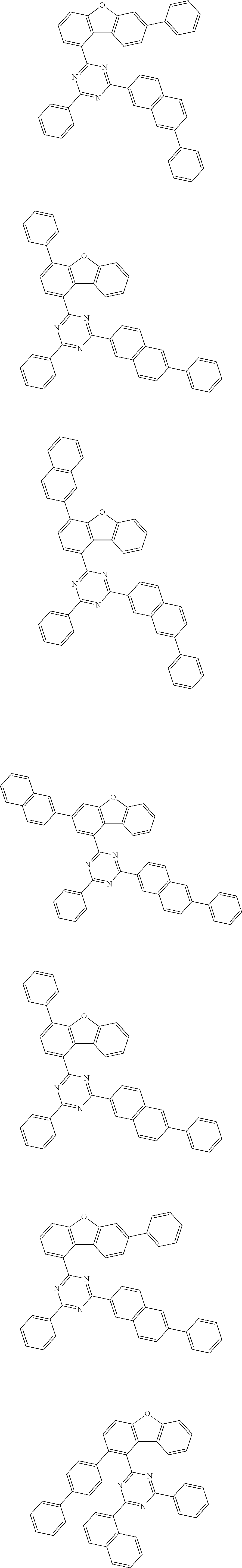

9. The organic light emitting device according to claim 1,
wherein Ar₃ is hydrogen or phenyl.

10. The organic light emitting device according to claim 1,
wherein Ar₄ and Ar₅ are each independently phenyl, phenyl substituted with 5 deuteriums, biphenylyl, biphenylyl substituted with 4 deuteriums, biphenylyl substituted with 9 deuteriums, terphenylyl, terphenylyl substituted with 4 deuteriums, quaterphenylyl, naphthyl, phenanthrenyl, triphenylenyl, dimethylfluorenyl, diphenylfluorenyl, carbazolyl, phenylcarbazolyl, dibenzofuranyl, dibenzothiophenyl, or phenyl dibenzofuranyl.

11. The organic light emitting device according to claim 1,
wherein Ar₄ and Ar₅ are each independently any one selected from the group consisting of the following:

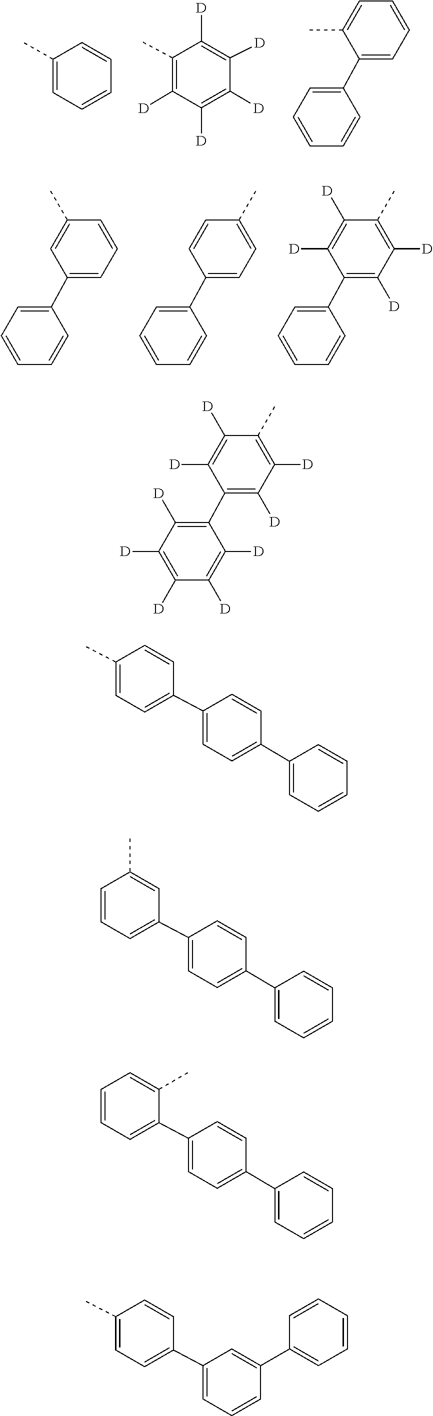

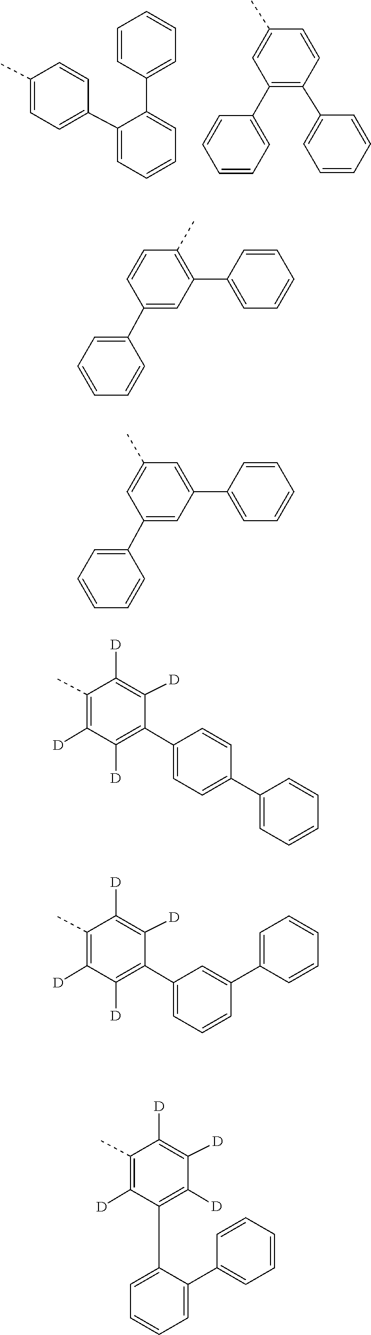

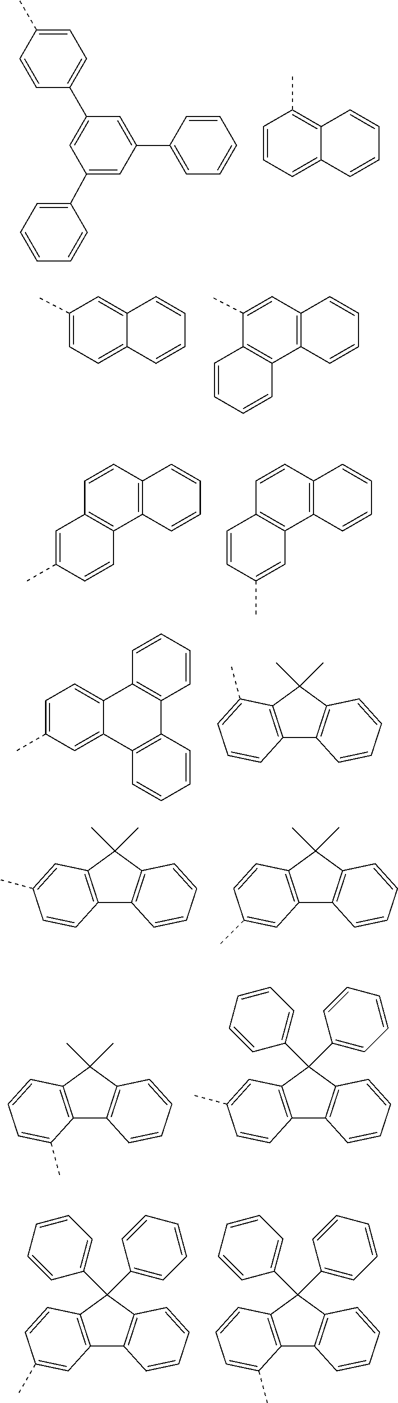

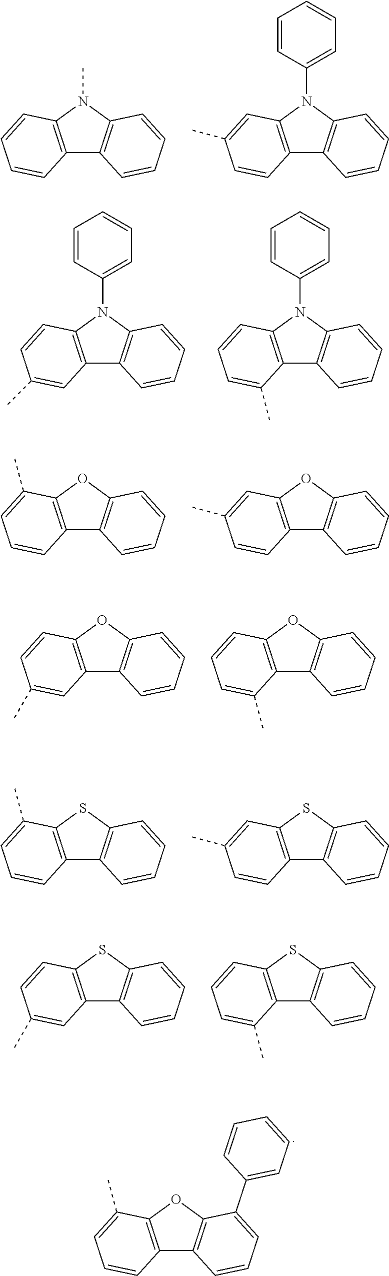

12. The organic light emitting device according to claim 1,
wherein L₄ to L₆ are each independently a single bond, phenylene, phenylene substituted with 4 deuteriums, biphenylylene, naphthylene, phenyl naphthylene, carbazolylene, phenyl carbazolylene, phenyl carbazolylene substituted with 4 deuteriums, dibenzofuranylene, phenyl dibenzofuranylene, phenyl dibenzofuranylene substituted with 4 deuteriums, or dimethylfluorenylene.

13. The organic light emitting device according to claim 1,
wherein L₄ to L₆ are each independently a single bond or any one selected from the group consisting of the following:

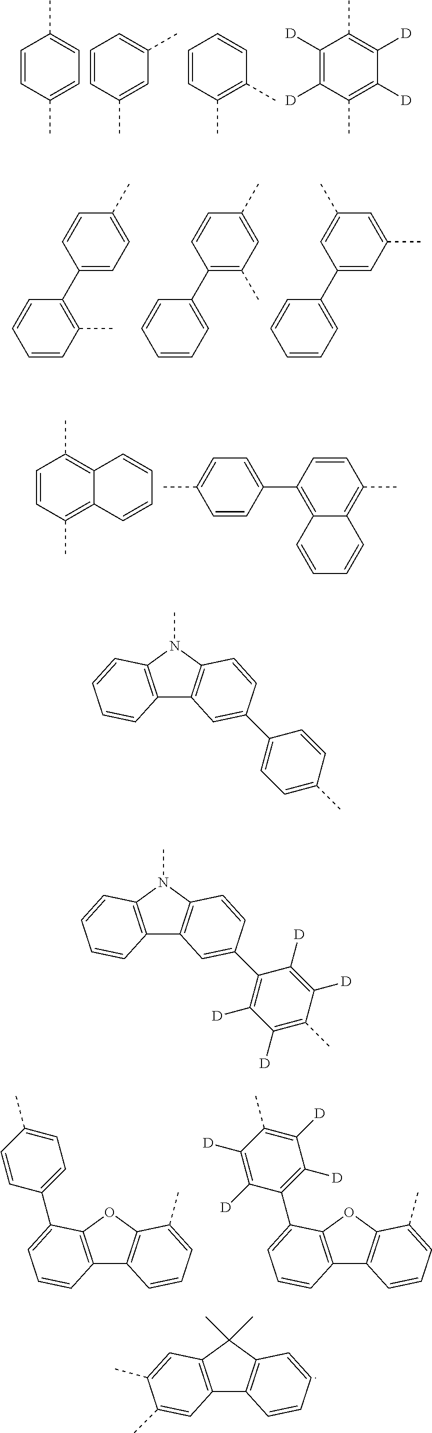

14. The organic light emitting device according to claim 1,
wherein L₇ is a substituted or unsubstituted phenylene, a substituted or unsubstituted biphenylylene, or a substituted or unsubstituted naphthylene.

15. The organic light emitting device according to claim 1,
wherein the compound of Chemical Formula 2 is the following Chemical Formula 2-1: 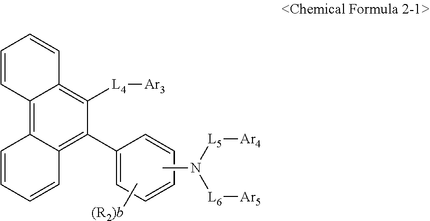
wherein in Chemical Formula 2-1:
Ar₃ to Ar₅ and L₄ to L₆ are as defined in claim 1;
R₂ is hydrogen, deuterium, or a substituted or unsubstituted C₆₋₆₀ aryl; and
b is an integer of 0 to 4.

16. The organic light emitting device according to claim 15,
wherein R₂ is hydrogen or deuterium.

17. The organic light emitting device according to claim 1,
wherein the compound of Chemical Formula 2 is selected from the group consisting of the following: 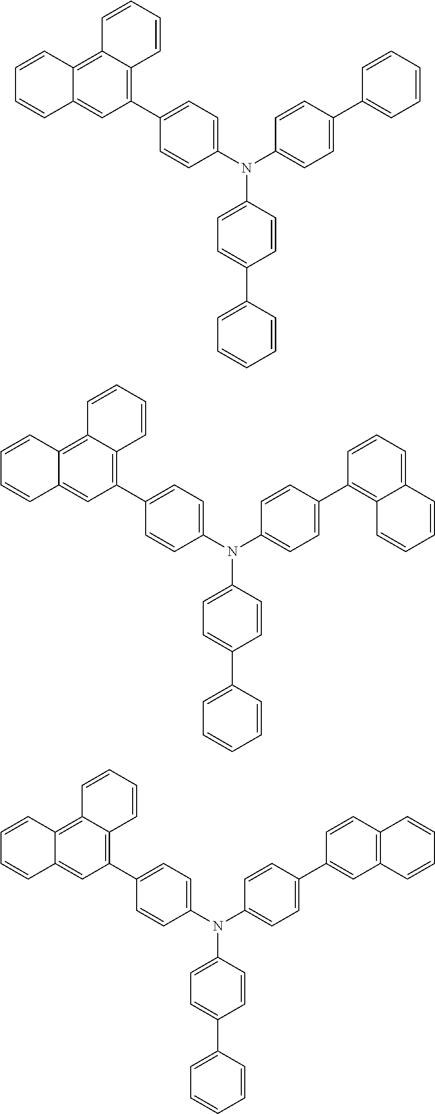 [SMILES: c1ccc(-c2ccc(N(c3ccc(-c4ccccc4)cc3)c3ccc(-c4cc5ccccc5c5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5ccccc5c4)cc3)c3ccc(-c4cc5ccccc5c5ccccc45)cc3)cc2)cc1] 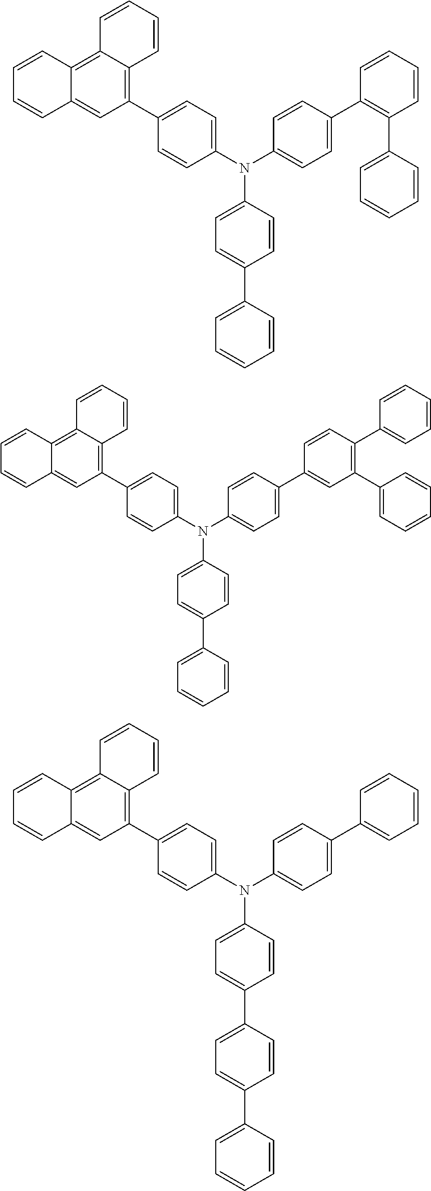 [SMILES: c1ccc(-c2ccc(N(c3ccc(-c4ccccc4-c4ccccc4)cc3)c3ccc(-c4cc5ccccc5c5ccccc45)cc3)cc2)cc1 ; c1ccc(-c2ccc(N(c3ccc(-c4ccc(-c5ccccc5)c(-c5ccccc5)c4)cc3)c3ccc(-c4cc5ccccc5c5ccccc45)cc3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1] 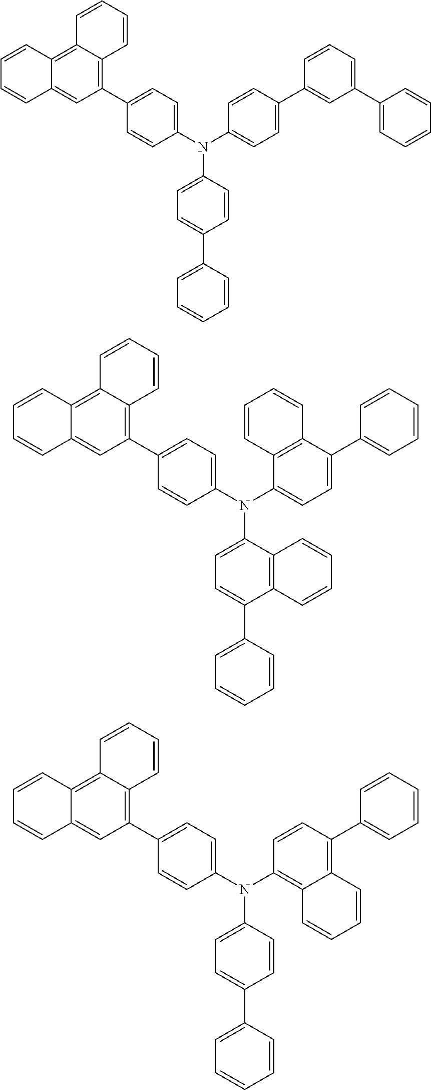 [SMILES: c1ccc(-c2ccc(N(c3ccc(-c4cccc(-c5ccccc5)c4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c4ccccc34)c3ccccc23)cc1 ; c1ccc(-c2ccc(N(c3ccc(-c4cc5ccccc5c5ccccc45)cc3)c3ccc(-c4ccccc4)c4ccccc34)cc2)cc1] 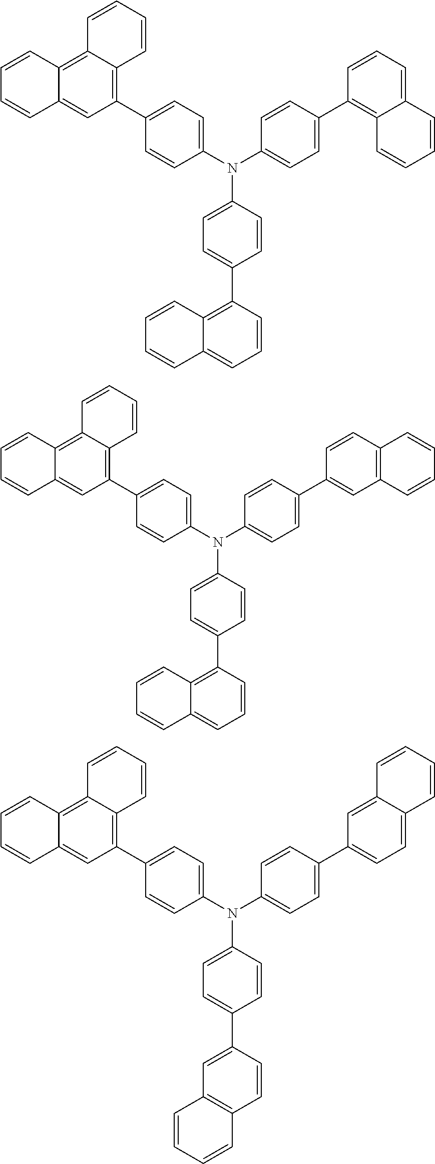 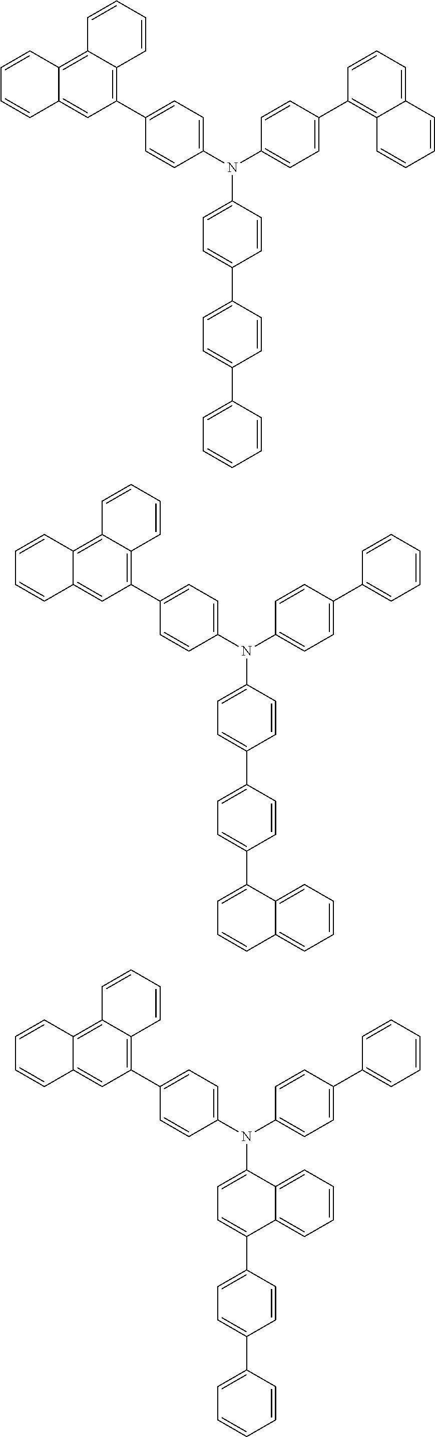 [SMILES: c1ccc(-c2ccc(-c3ccc(N(c4ccc(-c5cccc6ccccc56)cc4)c4ccc(-c5cc6ccccc6c6ccccc56)cc4)cc3)cc2)cc1 ; c1ccc(-c2ccc(N(c3ccc(-c4ccc(-c5cccc6ccccc56)cc4)cc3)c3ccc(-c4cc5ccccc5c5ccccc45)cc3)cc2)cc1 ; c1ccc(-c2ccc(-c3ccc(N(c4ccc(-c5ccccc5)cc4)c4ccc(-c5cc6ccccc6c6ccccc56)cc4)c4ccccc34)cc2)cc1] 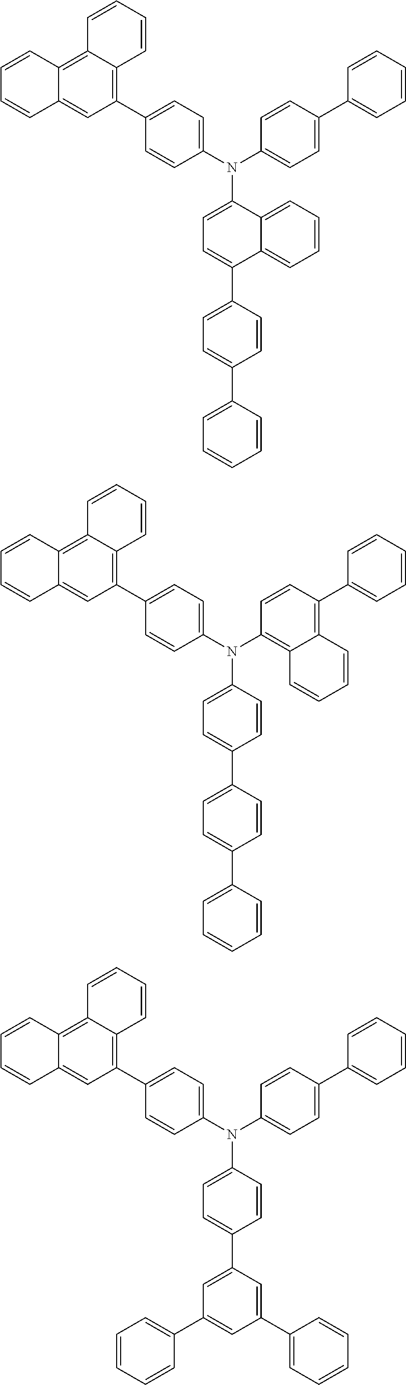 [SMILES: c1ccc(-c2ccc(-c3ccc(N(c4ccc(-c5ccccc5)cc4)c4ccc(-c5cc6ccccc6c6ccccc56)cc4)c4ccccc34)cc2)cc1 ; c1ccc(-c2ccc(-c3ccc(N(c4ccc(-c5cc6ccccc6c6ccccc56)cc4)c4ccc(-c5ccccc5)c5ccccc45)cc3)cc2)cc1 ; c1ccc(-c2ccc(N(c3ccc(-c4cc(-c5ccccc5)cc(-c5ccccc5)c4)cc3)c3ccc(-c4cc5ccccc5c5ccccc45)cc3)cc2)cc1] 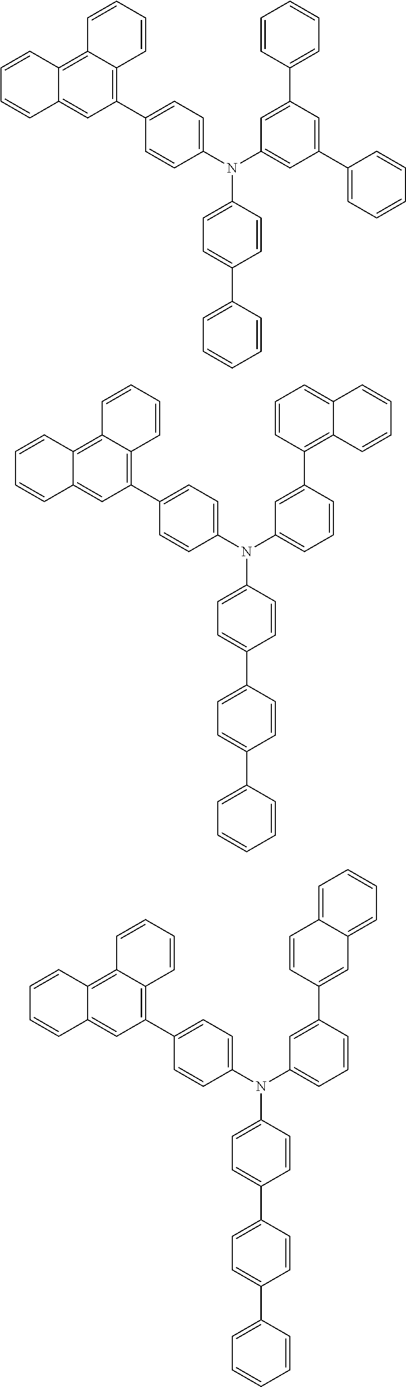 [SMILES: c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2ccc(-c3ccc(N(c4ccc(-c5cc6ccccc6c6ccccc56)cc4)c4cccc(-c5cccc6ccccc56)c4)cc3)cc2)cc1 ; c1ccc(-c2ccc(-c3ccc(N(c4ccc(-c5cc6ccccc6c6ccccc56)cc4)c4cccc(-c5ccc6ccccc6c5)c4)cc3)cc2)cc1] 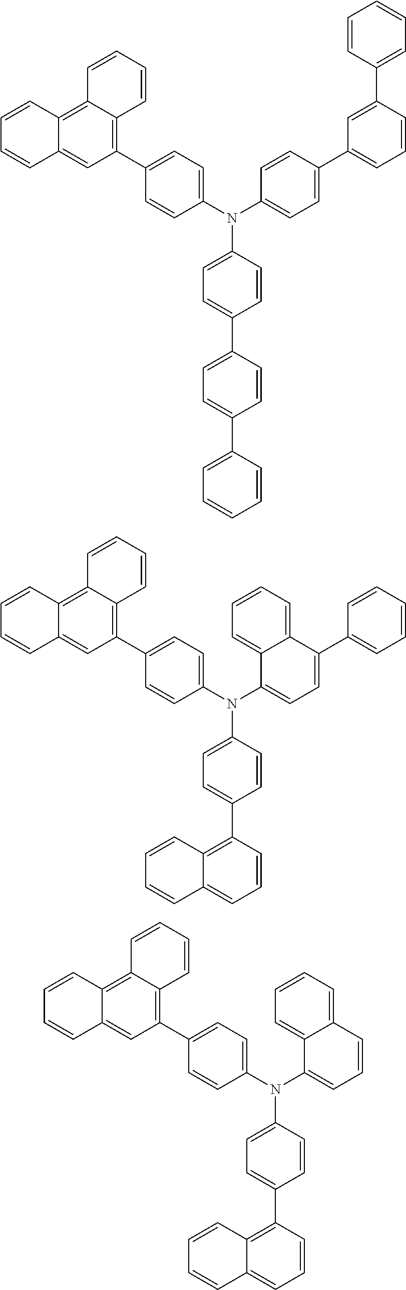 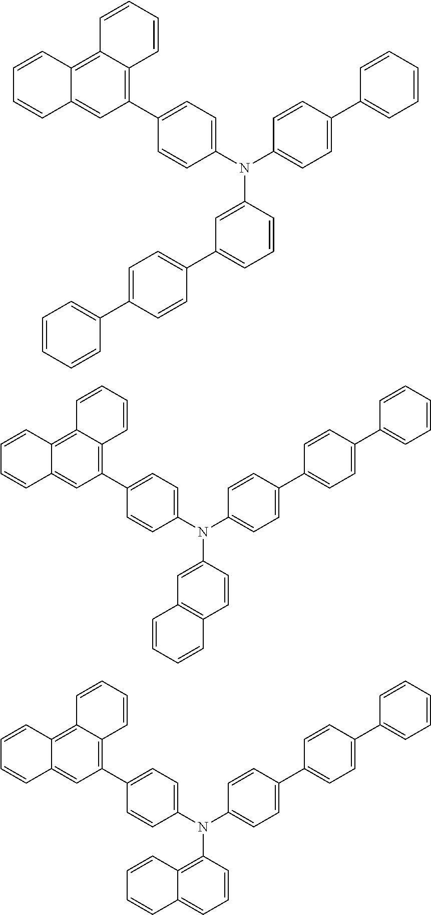 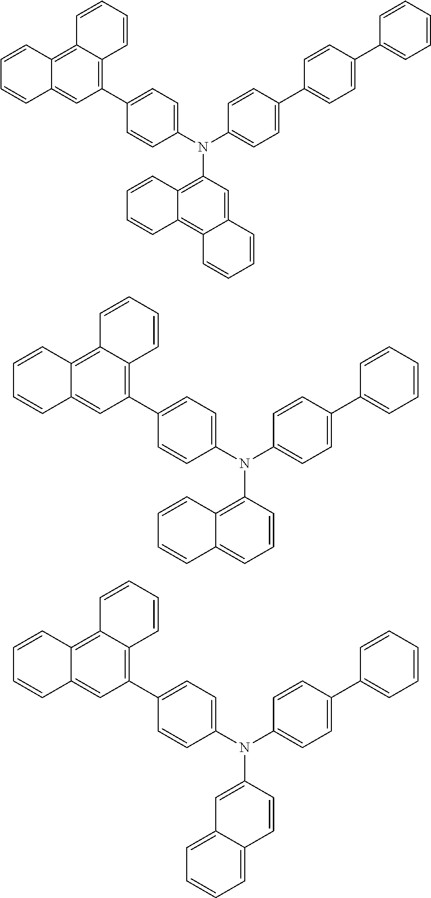 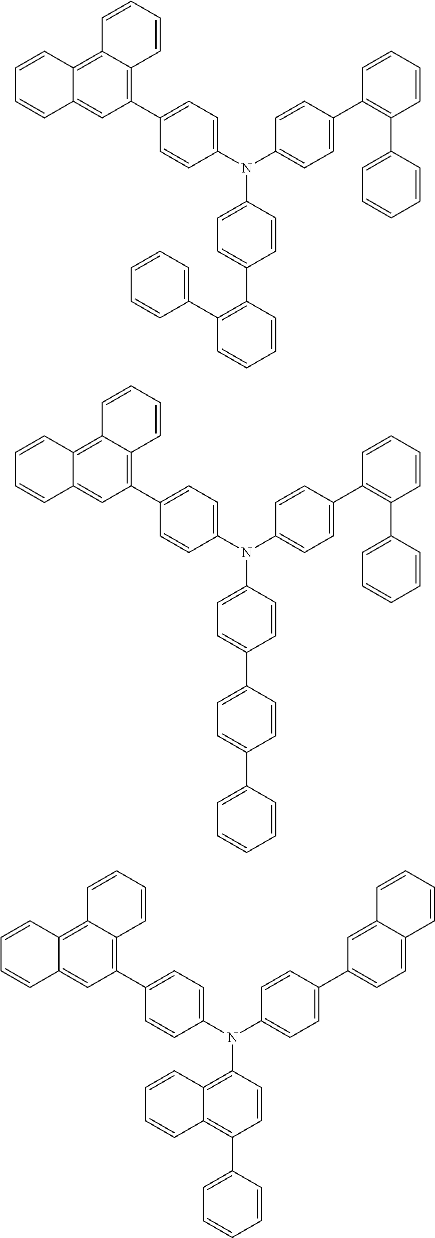 [SMILES: c1ccc(-c2ccccc2-c2ccc(N(c3ccc(-c4ccccc4-c4ccccc4)cc3)c3ccc(-c4cc5ccccc5c5ccccc45)cc3)cc2)cc1 ; c1ccc(-c2ccc(-c3ccc(N(c4ccc(-c5ccccc5-c5ccccc5)cc4)c4ccc(-c5cc6ccccc6c6ccccc56)cc4)cc3)cc2)cc1 ; c1ccc(-c2ccc(N(c3ccc(-c4ccc5ccccc5c4)cc3)c3ccc(-c4cc5ccccc5c5ccccc45)cc3)c3ccccc23)cc1] 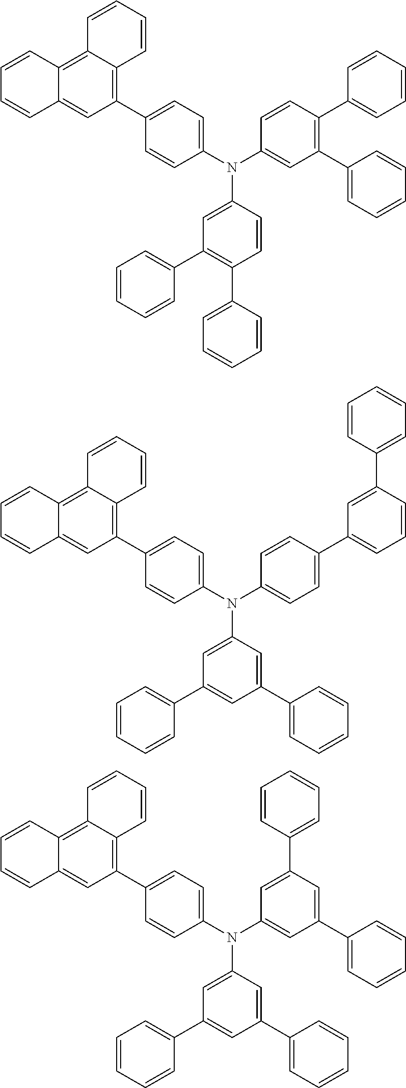 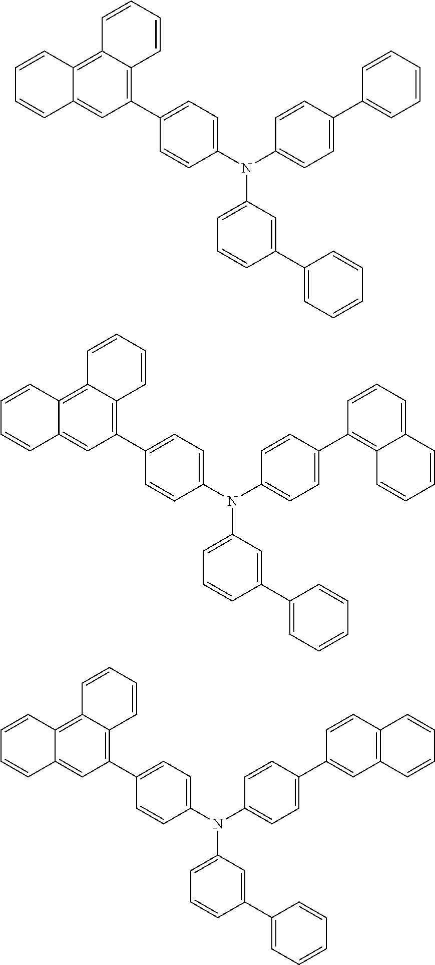 [SMILES: c1ccc(-c2ccc(N(c3ccc(-c4cc5ccccc5c5ccccc45)cc3)c3cccc(-c4ccccc4)c3)cc2)cc1 ; c1ccc(-c2cccc(N(c3ccc(-c4cccc5ccccc45)cc3)c3ccc(-c4cc5ccccc5c5ccccc45)cc3)c2)cc1 ; c1ccc(-c2cccc(N(c3ccc(-c4ccc5ccccc5c4)cc3)c3ccc(-c4cc5ccccc5c5ccccc45)cc3)c2)cc1] 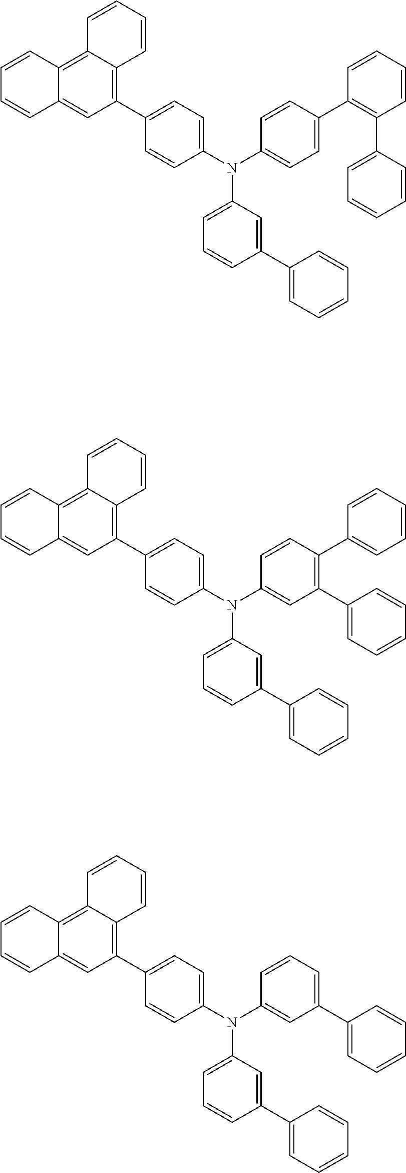 [SMILES: c1ccc(-c2cccc(N(c3ccc(-c4ccccc4-c4ccccc4)cc3)c3ccc(-c4cc5ccccc5c5ccccc45)cc3)c2)cc1 ; c1ccc(-c2cccc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cccc(-c4ccccc4)c3)c2)cc1] 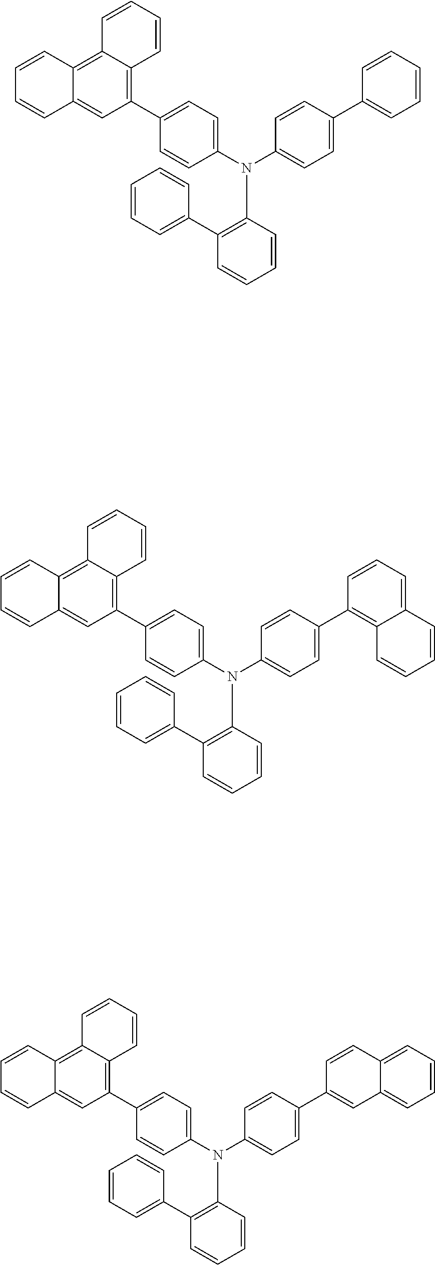 [SMILES: c1ccc(-c2ccc(N(c3ccc(-c4cc5ccccc5c5ccccc45)cc3)c3ccccc3-c3ccccc3)cc2)cc1 ; c1ccc(-c2ccccc2N(c2ccc(-c3cccc4ccccc34)cc2)c2ccc(-c3cc4ccccc4c4ccccc34)cc2)cc1 ; c1ccc(-c2ccccc2N(c2ccc(-c3ccc4ccccc4c3)cc2)c2ccc(-c3cc4ccccc4c4ccccc34)cc2)cc1] 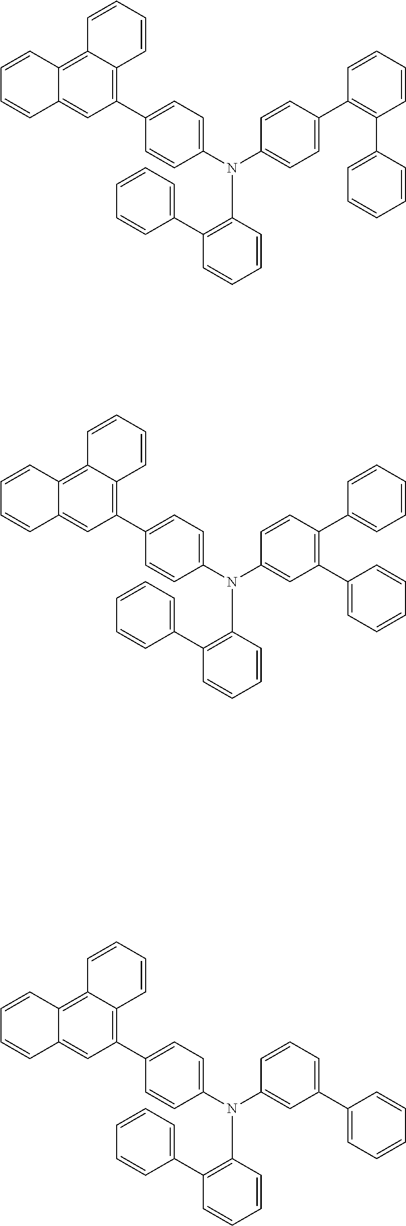 [SMILES: c1ccc(-c2ccccc2-c2ccc(N(c3ccc(-c4cc5ccccc5c5ccccc45)cc3)c3ccccc3-c3ccccc3)cc2)cc1 ; c1ccc(-c2ccc(N(c3ccc(-c4cc5ccccc5c5ccccc45)cc3)c3ccccc3-c3ccccc3)cc2-c2ccccc2)cc1 ; c1ccc(-c2cccc(N(c3ccc(-c4cc5ccccc5c5ccccc45)cc3)c3ccccc3-c3ccccc3)c2)cc1] 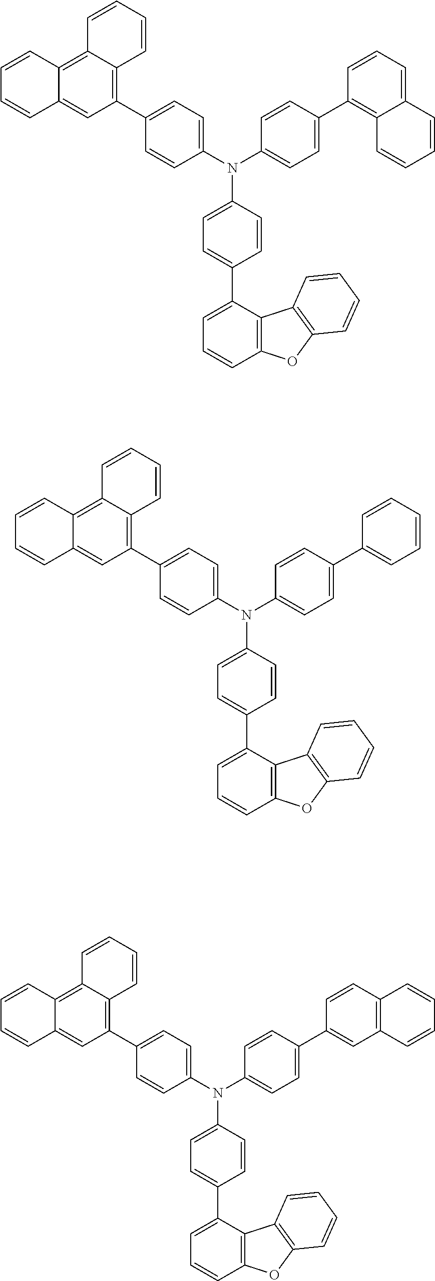 [SMILES: c1ccc2c(-c3ccc(N(c4ccc(-c5cc6ccccc6c6ccccc56)cc4)c4ccc(-c5cccc6oc7ccccc7c56)cc4)cc3)cccc2c1 ; c1ccc(-c2ccc(N(c3ccc(-c4cc5ccccc5c5ccccc45)cc3)c3ccc(-c4cccc5oc6ccccc6c45)cc3)cc2)cc1 ; c1ccc2cc(-c3ccc(N(c4ccc(-c5cc6ccccc6c6ccccc56)cc4)c4ccc(-c5cccc6oc7ccccc7c56)cc4)cc3)ccc2c1] 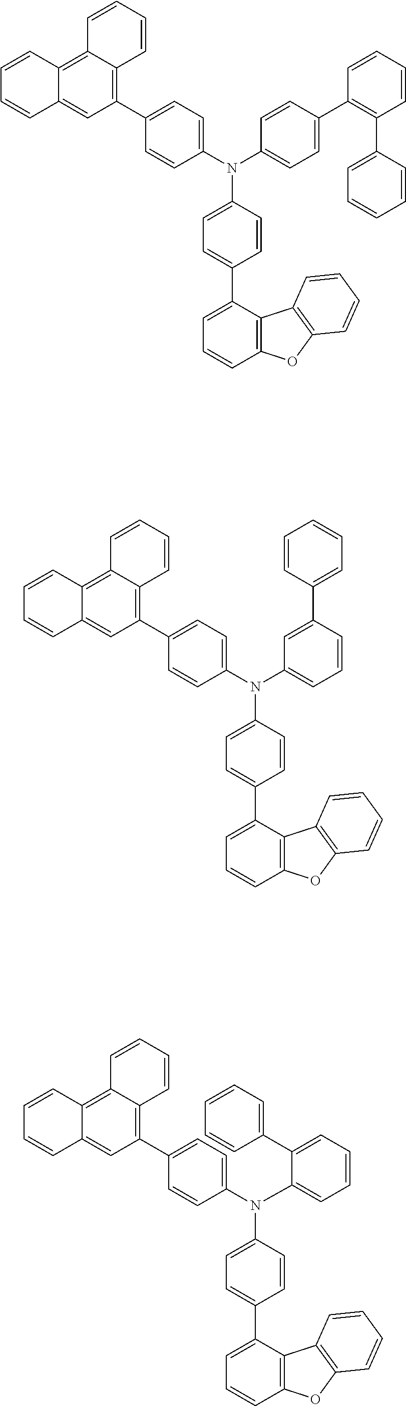 [SMILES: c1ccc(-c2ccccc2-c2ccc(N(c3ccc(-c4cc5ccccc5c5ccccc45)cc3)c3ccc(-c4cccc5oc6ccccc6c45)cc3)cc2)cc1 ; c1ccc(-c2cccc(N(c3ccc(-c4cc5ccccc5c5ccccc45)cc3)c3ccc(-c4cccc5oc6ccccc6c45)cc3)c2)cc1 ; c1ccc(-c2ccccc2N(c2ccc(-c3cc4ccccc4c4ccccc34)cc2)c2ccc(-c3cccc4oc5ccccc5c34)cc2)cc1] 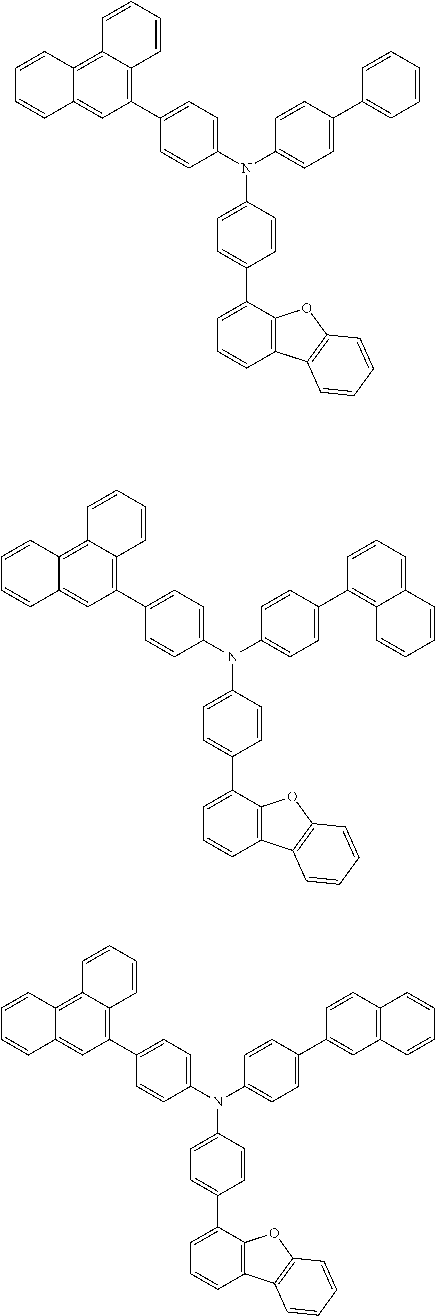 [SMILES: c1ccc(-c2ccc(N(c3ccc(-c4cc5ccccc5c5ccccc45)cc3)c3ccc(-c4cccc5c4oc4ccccc45)cc3)cc2)cc1 ; c1ccc2c(-c3ccc(N(c4ccc(-c5cc6ccccc6c6ccccc56)cc4)c4ccc(-c5cccc6c5oc5ccccc56)cc4)cc3)cccc2c1 ; c1ccc2cc(-c3ccc(N(c4ccc(-c5cc6ccccc6c6ccccc56)cc4)c4ccc(-c5cccc6c5oc5ccccc56)cc4)cc3)ccc2c1] 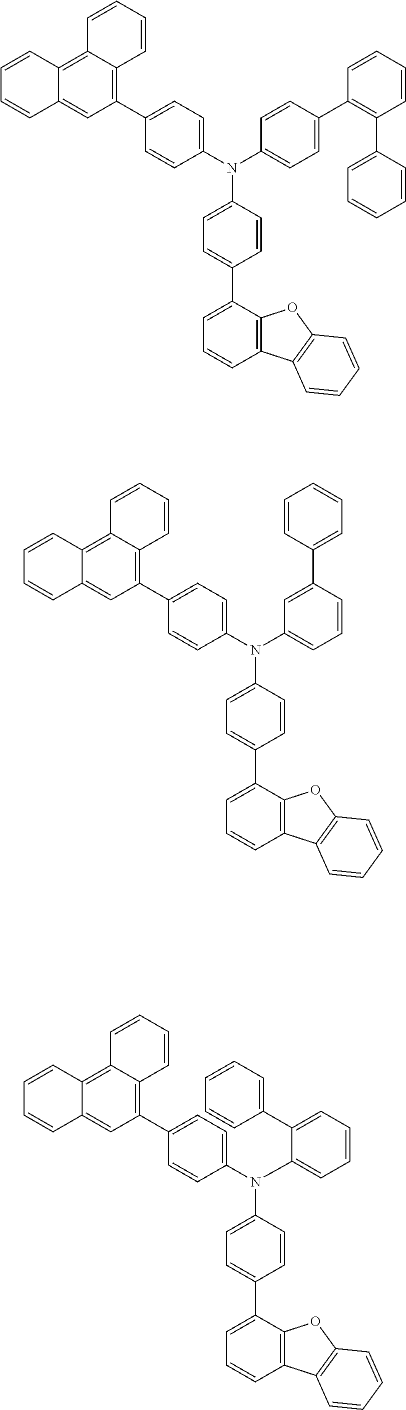 [SMILES: c1ccc(-c2ccccc2-c2ccc(N(c3ccc(-c4cc5ccccc5c5ccccc45)cc3)c3ccc(-c4cccc5c4oc4ccccc45)cc3)cc2)cc1 ; c1ccc(-c2cccc(N(c3ccc(-c4cc5ccccc5c5ccccc45)cc3)c3ccc(-c4cccc5c4oc4ccccc45)cc3)c2)cc1 ; c1ccc(-c2ccccc2N(c2ccc(-c3cc4ccccc4c4ccccc34)cc2)c2ccc(-c3cccc4c3oc3ccccc34)cc2)cc1] 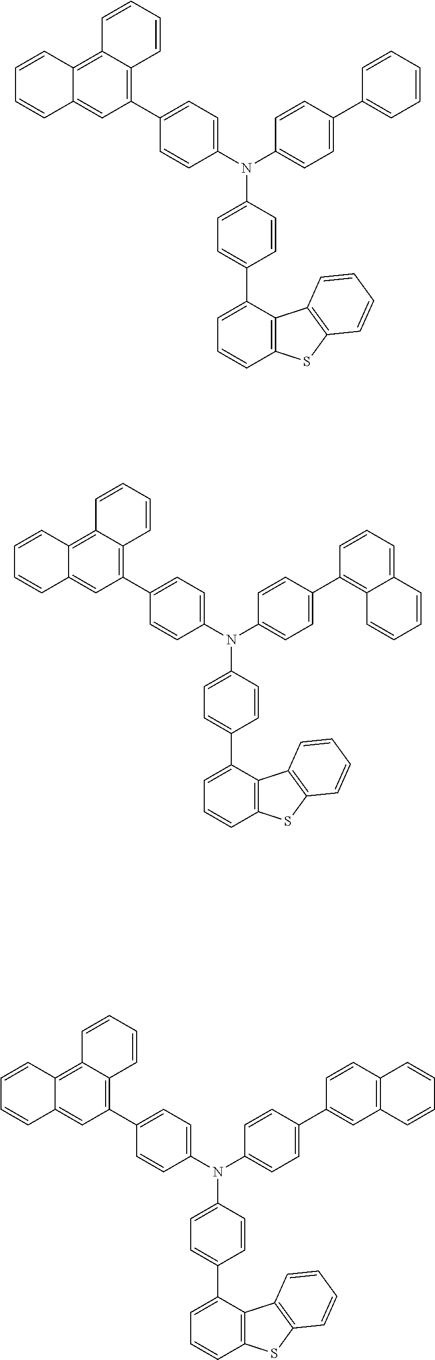 [SMILES: c1ccc(-c2ccc(N(c3ccc(-c4cc5ccccc5c5ccccc45)cc3)c3ccc(-c4cccc5sc6ccccc6c45)cc3)cc2)cc1 ; c1ccc2c(-c3ccc(N(c4ccc(-c5cc6ccccc6c6ccccc56)cc4)c4ccc(-c5cccc6sc7ccccc7c56)cc4)cc3)cccc2c1 ; c1ccc2cc(-c3ccc(N(c4ccc(-c5cc6ccccc6c6ccccc56)cc4)c4ccc(-c5cccc6sc7ccccc7c56)cc4)cc3)ccc2c1] 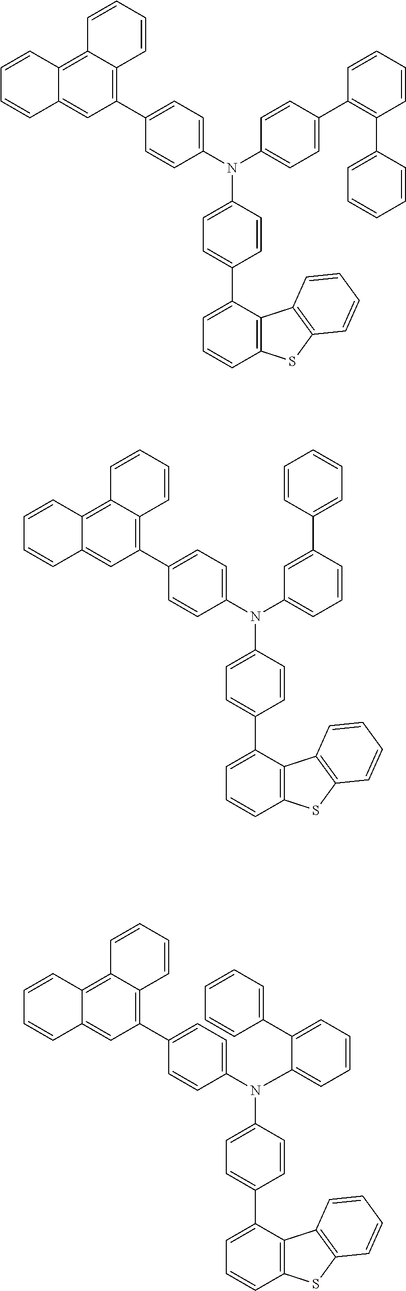 [SMILES: c1ccc(-c2ccccc2-c2ccc(N(c3ccc(-c4cc5ccccc5c5ccccc45)cc3)c3ccc(-c4cccc5sc6ccccc6c45)cc3)cc2)cc1 ; c1ccc(-c2cccc(N(c3ccc(-c4cc5ccccc5c5ccccc45)cc3)c3ccc(-c4cccc5sc6ccccc6c45)cc3)c2)cc1 ; c1ccc(-c2ccccc2N(c2ccc(-c3cc4ccccc4c4ccccc34)cc2)c2ccc(-c3cccc4sc5ccccc5c34)cc2)cc1] 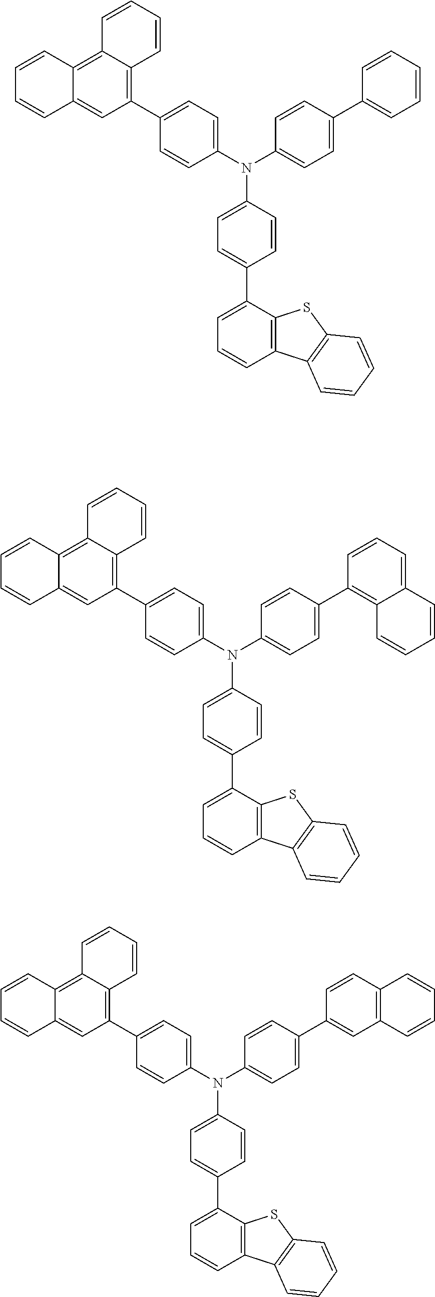 [SMILES: c1ccc(-c2ccc(N(c3ccc(-c4cc5ccccc5c5ccccc45)cc3)c3ccc(-c4cccc5c4sc4ccccc45)cc3)cc2)cc1 ; c1ccc2c(-c3ccc(N(c4ccc(-c5cc6ccccc6c6ccccc56)cc4)c4ccc(-c5cccc6c5sc5ccccc56)cc4)cc3)cccc2c1 ; c1ccc2cc(-c3ccc(N(c4ccc(-c5cc6ccccc6c6ccccc56)cc4)c4ccc(-c5cccc6c5sc5ccccc56)cc4)cc3)ccc2c1] 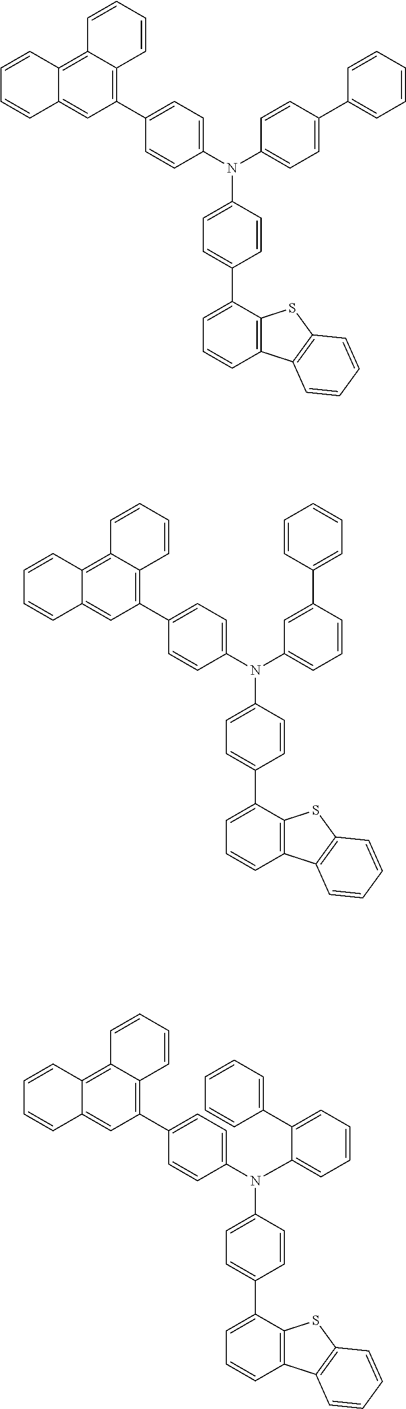 [SMILES: c1ccc(-c2ccc(N(c3ccc(-c4cc5ccccc5c5ccccc45)cc3)c3ccc(-c4cccc5c4sc4ccccc45)cc3)cc2)cc1 ; c1ccc(-c2cccc(N(c3ccc(-c4cc5ccccc5c5ccccc45)cc3)c3ccc(-c4cccc5c4sc4ccccc45)cc3)c2)cc1 ; c1ccc(-c2ccccc2N(c2ccc(-c3cc4ccccc4c4ccccc34)cc2)c2ccc(-c3cccc4c3sc3ccccc34)cc2)cc1] 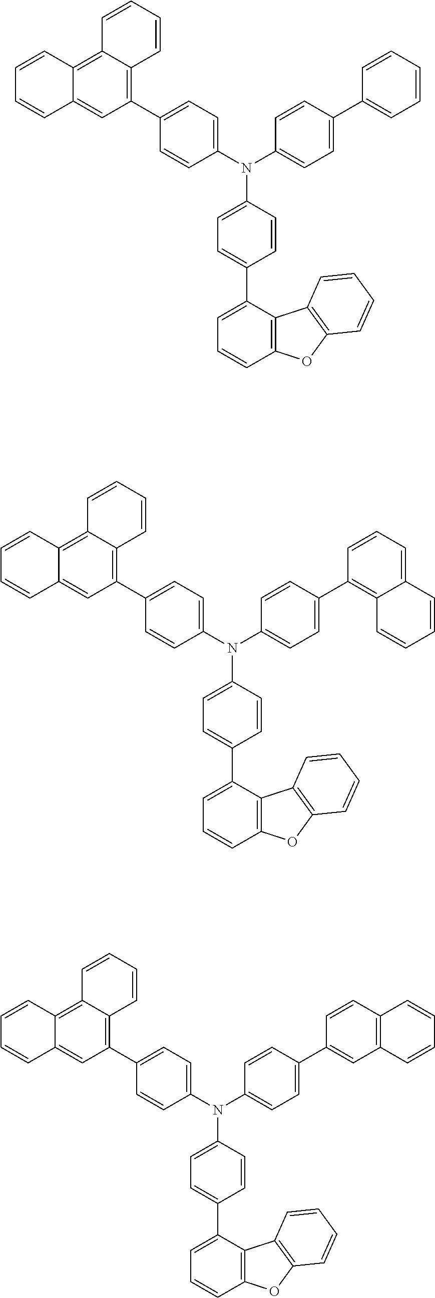 [SMILES: c1ccc(-c2ccc(N(c3ccc(-c4cc5ccccc5c5ccccc45)cc3)c3ccc(-c4cccc5oc6ccccc6c45)cc3)cc2)cc1 ; c1ccc2c(-c3ccc(N(c4ccc(-c5cc6ccccc6c6ccccc56)cc4)c4ccc(-c5cccc6oc7ccccc7c56)cc4)cc3)cccc2c1 ; c1ccc2cc(-c3ccc(N(c4ccc(-c5cc6ccccc6c6ccccc56)cc4)c4ccc(-c5cccc6oc7ccccc7c56)cc4)cc3)ccc2c1] 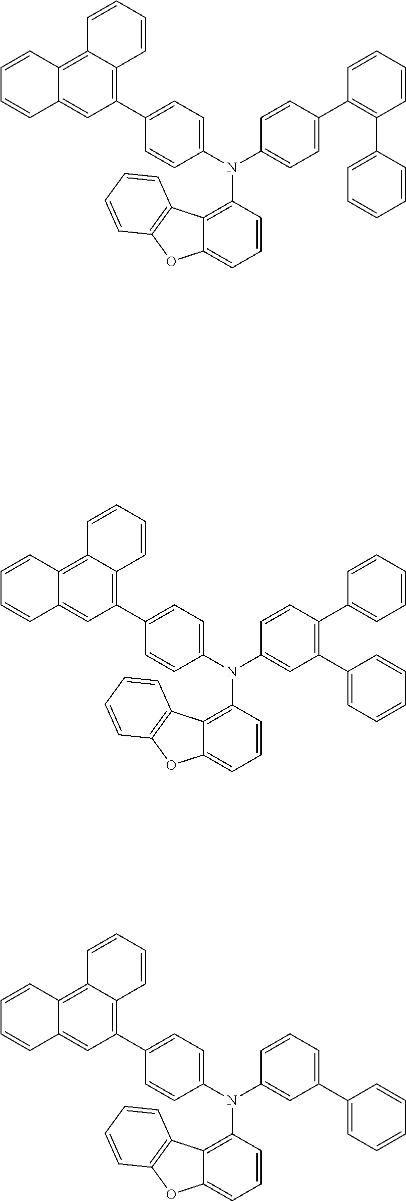 [SMILES: c1ccc(-c2ccccc2-c2ccc(N(c3ccc(-c4cc5ccccc5c5ccccc45)cc3)c3cccc4oc5ccccc5c34)cc2)cc1 ; c1ccc(-c2ccc(N(c3ccc(-c4cc5ccccc5c5ccccc45)cc3)c3cccc4oc5ccccc5c34)cc2-c2ccccc2)cc1 ; c1ccc(-c2cccc(N(c3ccc(-c4cc5ccccc5c5ccccc45)cc3)c3cccc4oc5ccccc5c34)c2)cc1] 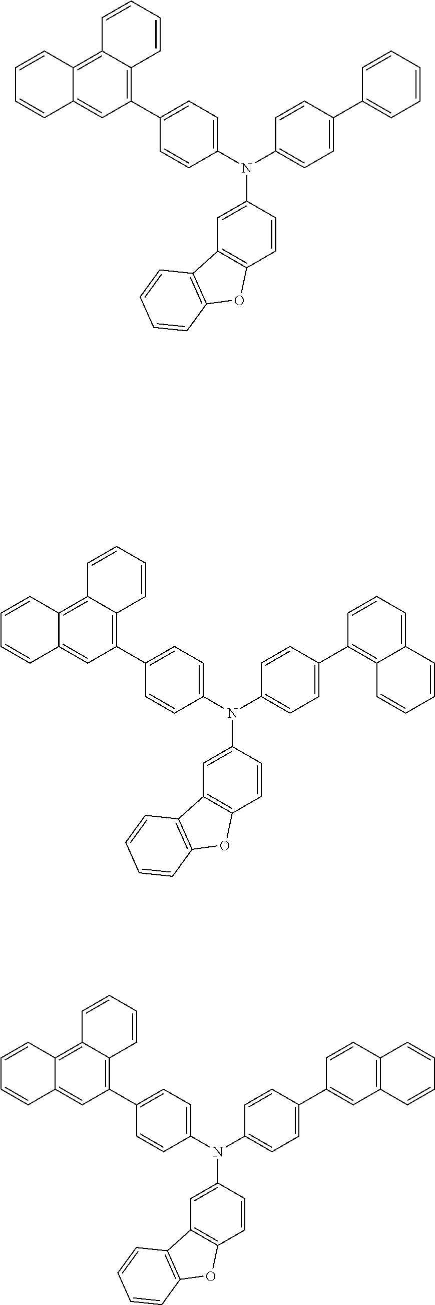 [SMILES: c1ccc(-c2ccc(N(c3ccc(-c4cc5ccccc5c5ccccc45)cc3)c3ccc4oc5ccccc5c4c3)cc2)cc1 ; c1ccc2c(-c3ccc(N(c4ccc(-c5cc6ccccc6c6ccccc56)cc4)c4ccc5oc6ccccc6c5c4)cc3)cccc2c1 ; c1ccc2cc(-c3ccc(N(c4ccc(-c5cc6ccccc6c6ccccc56)cc4)c4ccc5oc6ccccc6c5c4)cc3)ccc2c1] 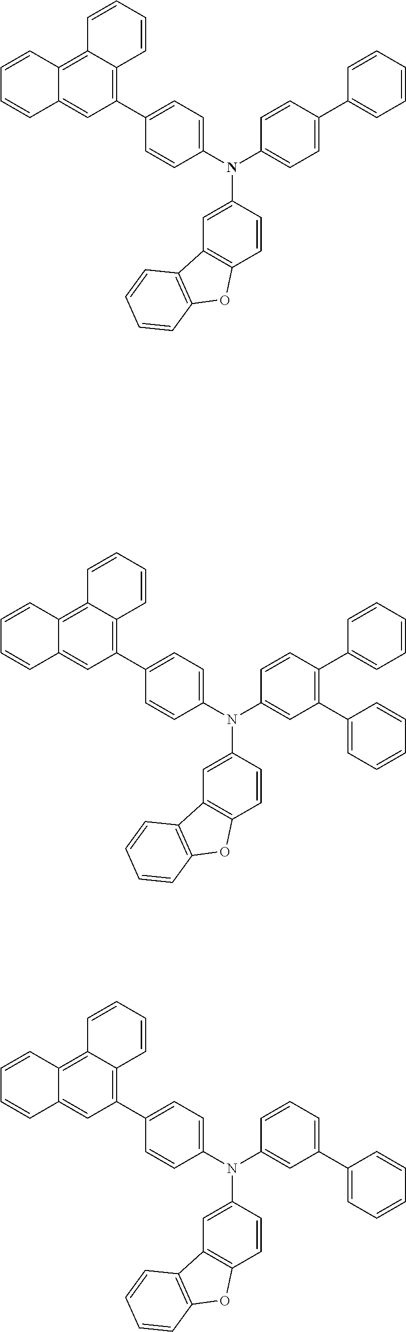 [SMILES: c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4oc5ccccc5c4c3)cc2-c2ccccc2)cc1 ; c1ccc(-c2cccc(N(c3ccc(-c4cc5ccccc5c5ccccc45)cc3)c3ccc4oc5ccccc5c4c3)c2)cc1] 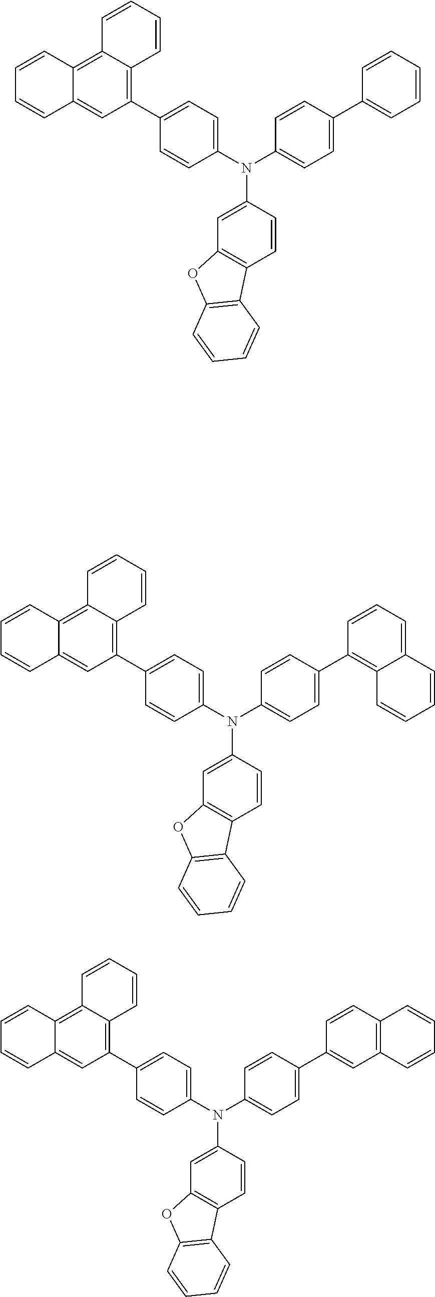 [SMILES: c1ccc(-c2ccc(N(c3ccc(-c4cc5ccccc5c5ccccc45)cc3)c3ccc4c(c3)oc3ccccc34)cc2)cc1 ; c1ccc2c(-c3ccc(N(c4ccc(-c5cc6ccccc6c6ccccc56)cc4)c4ccc5c(c4)oc4ccccc45)cc3)cccc2c1 ; c1ccc2cc(-c3ccc(N(c4ccc(-c5cc6ccccc6c6ccccc56)cc4)c4ccc5c(c4)oc4ccccc45)cc3)ccc2c1] 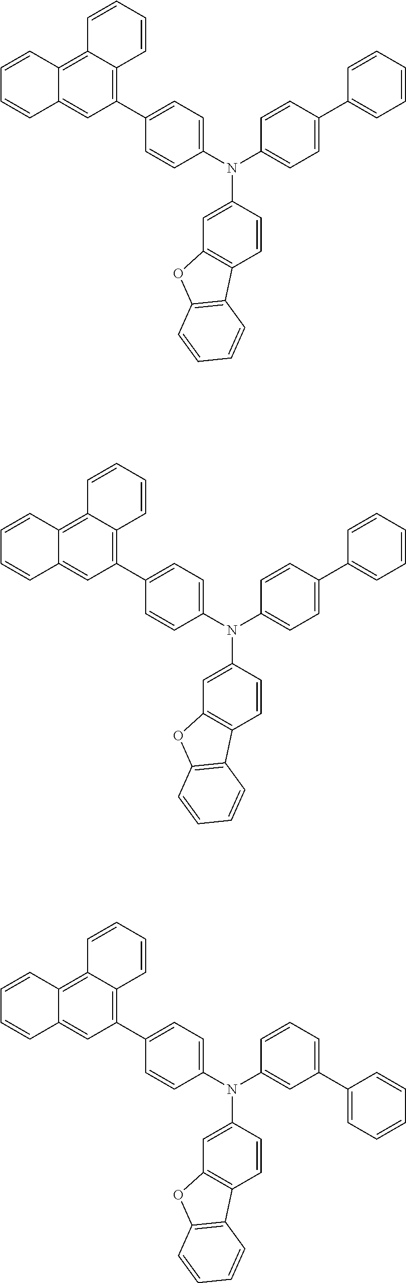 [SMILES: c1ccc(-c2ccc(N(c3ccc(-c4cc5ccccc5c5ccccc45)cc3)c3ccc4c(c3)oc3ccccc34)cc2)cc1 ; c1ccc(-c2ccc(N(c3ccc(-c4cc5ccccc5c5ccccc45)cc3)c3ccc4c(c3)oc3ccccc34)cc2)cc1 ; c1ccc(-c2cccc(N(c3ccc(-c4cc5ccccc5c5ccccc45)cc3)c3ccc4c(c3)oc3ccccc34)c2)cc1] 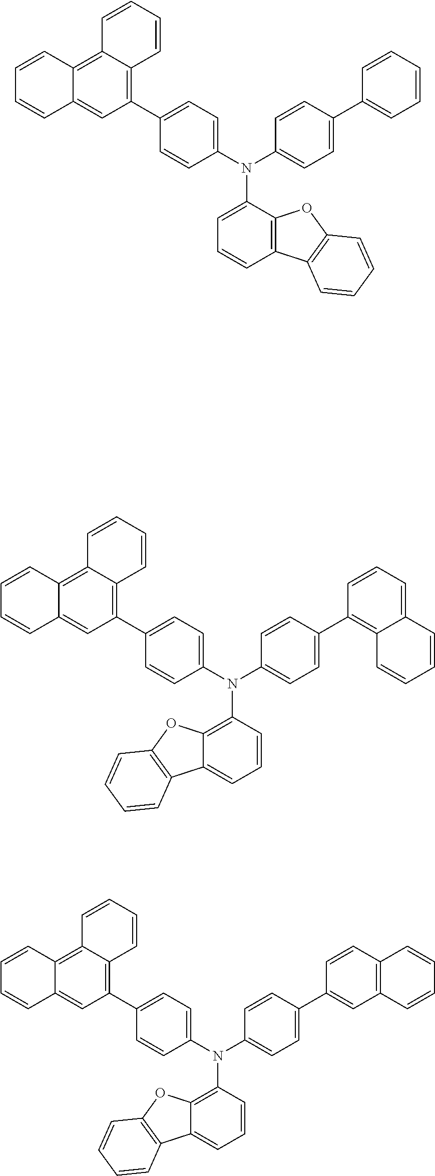 [SMILES: c1ccc(-c2ccc(N(c3ccc(-c4cc5ccccc5c5ccccc45)cc3)c3cccc4c3oc3ccccc34)cc2)cc1 ; c1ccc2c(-c3ccc(N(c4ccc(-c5cc6ccccc6c6ccccc56)cc4)c4cccc5c4oc4ccccc45)cc3)cccc2c1 ; c1ccc2cc(-c3ccc(N(c4ccc(-c5cc6ccccc6c6ccccc56)cc4)c4cccc5c4oc4ccccc45)cc3)ccc2c1] 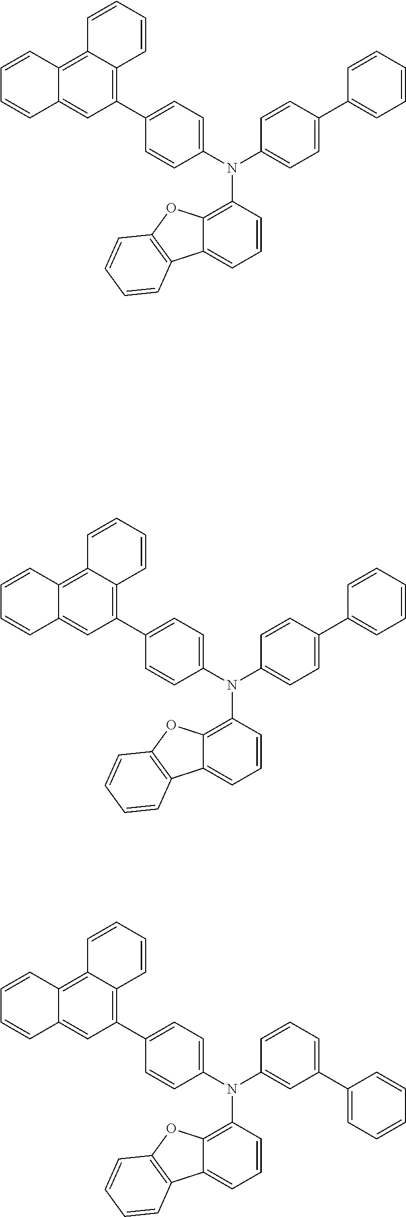 [SMILES: c1ccc(-c2ccc(N(c3ccc(-c4cc5ccccc5c5ccccc45)cc3)c3cccc4c3oc3ccccc34)cc2)cc1 ; c1ccc(-c2ccc(N(c3ccc(-c4cc5ccccc5c5ccccc45)cc3)c3cccc4c3oc3ccccc34)cc2)cc1 ; c1ccc(-c2cccc(N(c3ccc(-c4cc5ccccc5c5ccccc45)cc3)c3cccc4c3oc3ccccc34)c2)cc1] 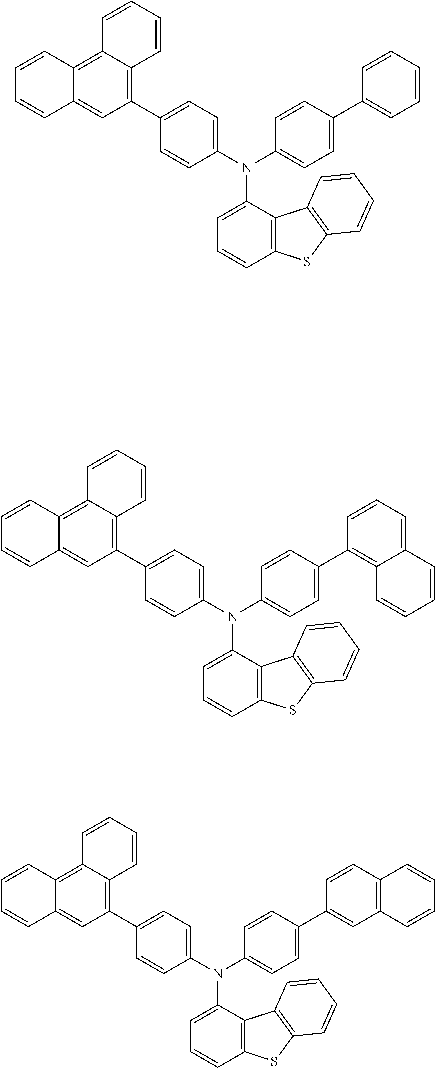 [SMILES: c1ccc(-c2ccc(N(c3ccc(-c4cc5ccccc5c5ccccc45)cc3)c3cccc4sc5ccccc5c34)cc2)cc1 ; c1ccc2c(-c3ccc(N(c4ccc(-c5cc6ccccc6c6ccccc56)cc4)c4cccc5sc6ccccc6c45)cc3)cccc2c1 ; c1ccc2cc(-c3ccc(N(c4ccc(-c5cc6ccccc6c6ccccc56)cc4)c4cccc5sc6ccccc6c45)cc3)ccc2c1] 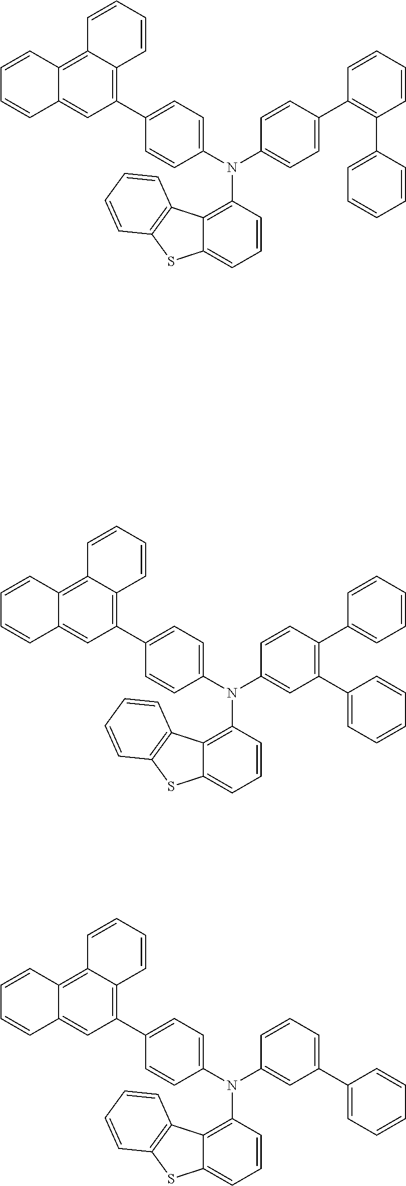 [SMILES: c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)cc2-c2ccccc2)cc1 ; c1ccc(-c2cccc(N(c3ccc(-c4cc5ccccc5c5ccccc45)cc3)c3cccc4sc5ccccc5c34)c2)cc1] 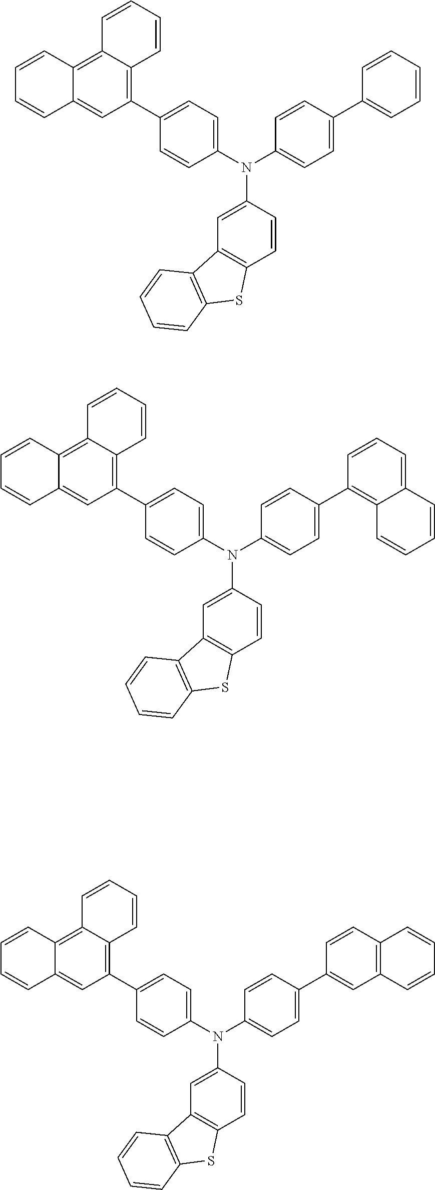 [SMILES: c1ccc(-c2ccc(N(c3ccc(-c4cc5ccccc5c5ccccc45)cc3)c3ccc4sc5ccccc5c4c3)cc2)cc1 ; c1ccc2c(-c3ccc(N(c4ccc(-c5cc6ccccc6c6ccccc56)cc4)c4ccc5sc6ccccc6c5c4)cc3)cccc2c1 ; c1ccc2cc(-c3ccc(N(c4ccc(-c5cc6ccccc6c6ccccc56)cc4)c4ccc5sc6ccccc6c5c4)cc3)ccc2c1] 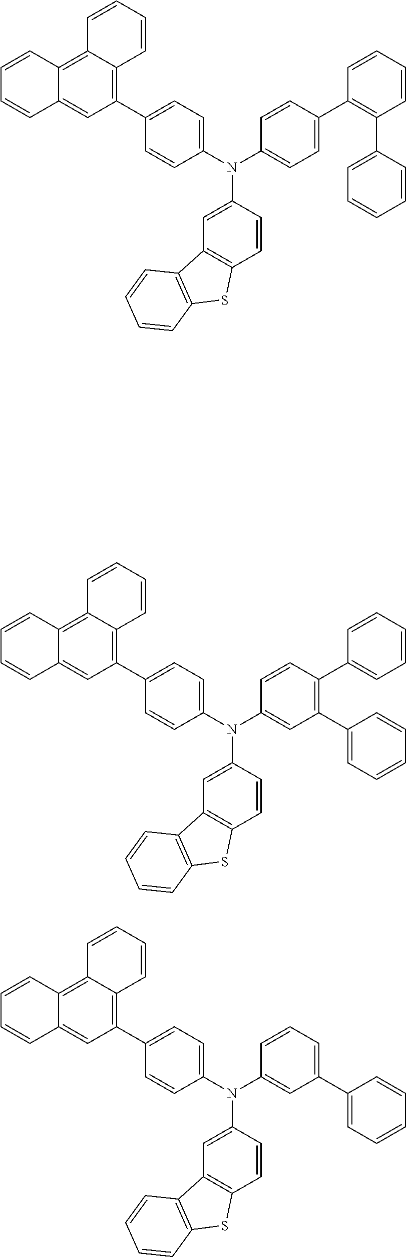 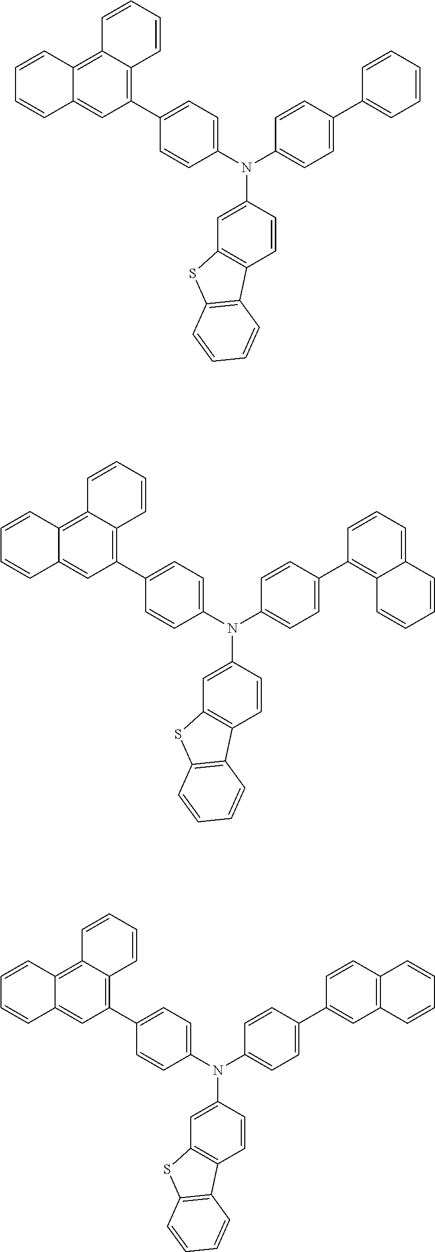 [SMILES: c1ccc(-c2ccc(N(c3ccc(-c4cc5ccccc5c5ccccc45)cc3)c3ccc4c(c3)sc3ccccc34)cc2)cc1 ; c1ccc2c(-c3ccc(N(c4ccc(-c5cc6ccccc6c6ccccc56)cc4)c4ccc5c(c4)sc4ccccc45)cc3)cccc2c1 ; c1ccc2cc(-c3ccc(N(c4ccc(-c5cc6ccccc6c6ccccc56)cc4)c4ccc5c(c4)sc4ccccc45)cc3)ccc2c1] 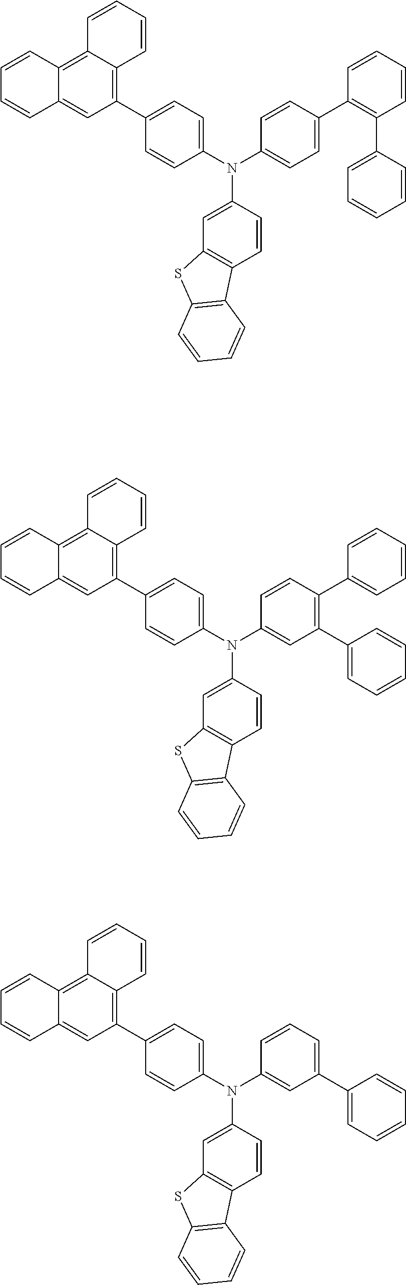 [SMILES: c1ccc(-c2ccccc2-c2ccc(N(c3ccc(-c4cc5ccccc5c5ccccc45)cc3)c3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2-c2ccccc2)cc1 ; c1ccc(-c2cccc(N(c3ccc(-c4cc5ccccc5c5ccccc45)cc3)c3ccc4c(c3)sc3ccccc34)c2)cc1] 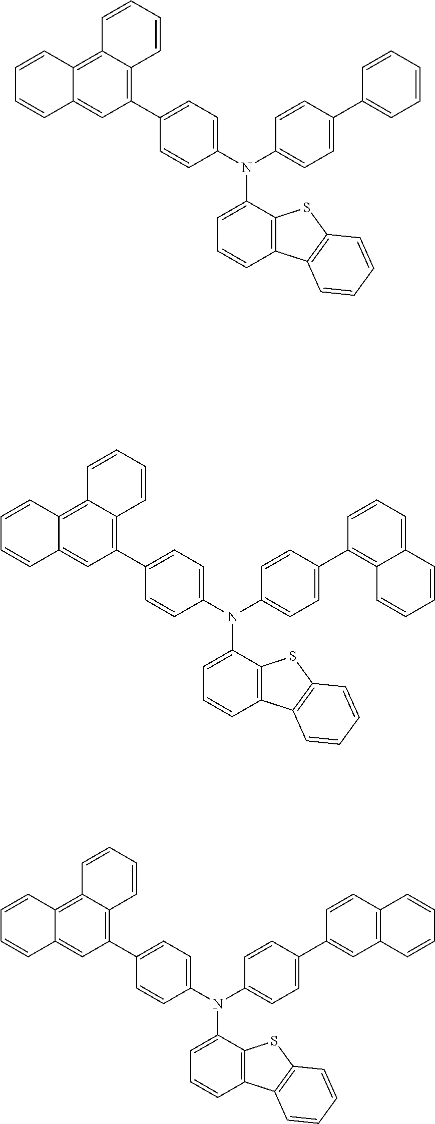 [SMILES: c1ccc(-c2ccc(N(c3ccc(-c4cc5ccccc5c5ccccc45)cc3)c3cccc4c3sc3ccccc34)cc2)cc1 ; c1ccc2c(-c3ccc(N(c4ccc(-c5cc6ccccc6c6ccccc56)cc4)c4cccc5c4sc4ccccc45)cc3)cccc2c1 ; c1ccc2cc(-c3ccc(N(c4ccc(-c5cc6ccccc6c6ccccc56)cc4)c4cccc5c4sc4ccccc45)cc3)ccc2c1] 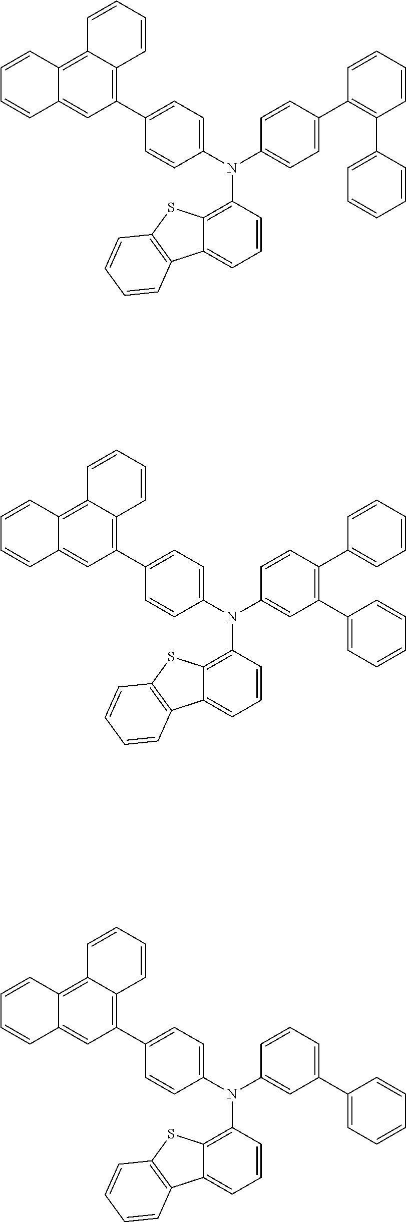 [SMILES: c1ccc(-c2ccccc2-c2ccc(N(c3ccc(-c4cc5ccccc5c5ccccc45)cc3)c3cccc4c3sc3ccccc34)cc2)cc1 ; c1ccc(-c2ccc(N(c3ccc(-c4cc5ccccc5c5ccccc45)cc3)c3cccc4c3sc3ccccc34)cc2-c2ccccc2)cc1 ; c1ccc(-c2cccc(N(c3ccc(-c4cc5ccccc5c5ccccc45)cc3)c3cccc4c3sc3ccccc34)c2)cc1] 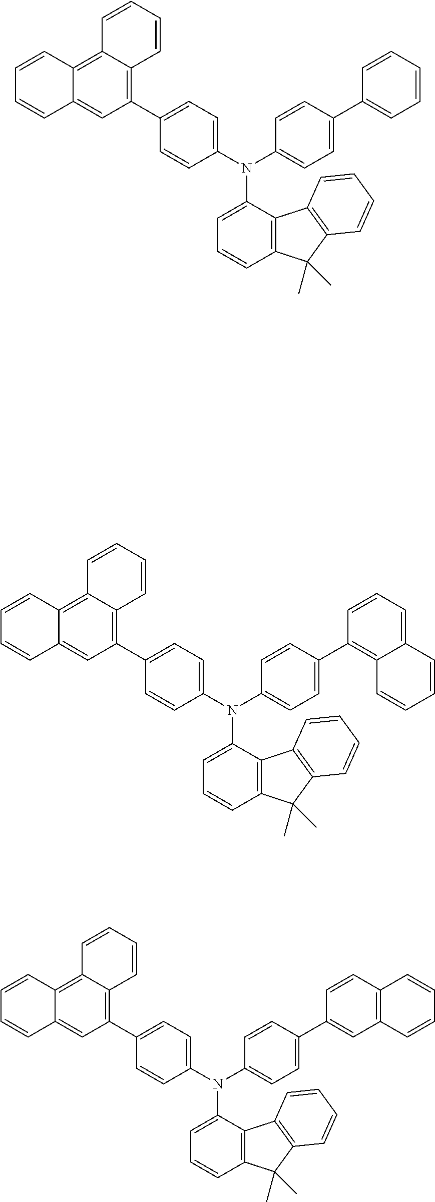 [SMILES: CC1(C)c2ccccc2-c2c(N(c3ccc(-c4ccccc4)cc3)c3ccc(-c4cc5ccccc5c5ccccc45)cc3)cccc21 ; CC1(C)c2ccccc2-c2c(N(c3ccc(-c4cccc5ccccc45)cc3)c3ccc(-c4cc5ccccc5c5ccccc45)cc3)cccc21 ; CC1(C)c2ccccc2-c2c(N(c3ccc(-c4ccc5ccccc5c4)cc3)c3ccc(-c4cc5ccccc5c5ccccc45)cc3)cccc21] 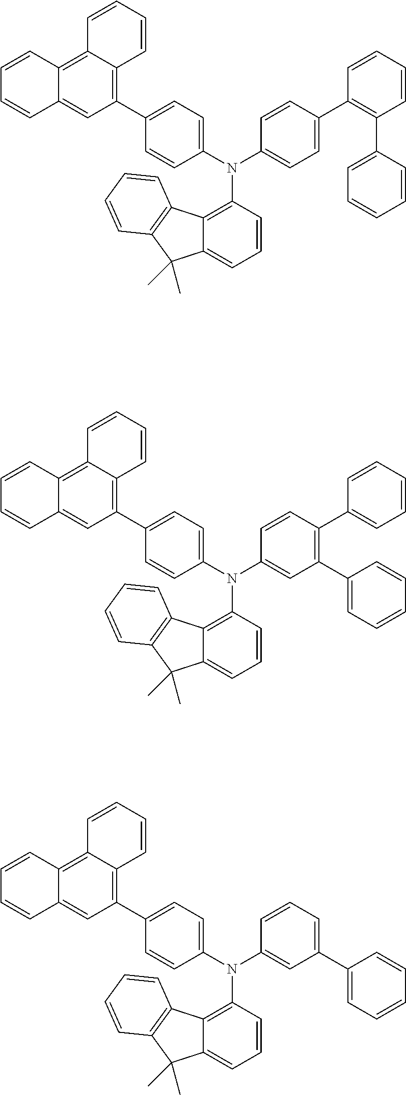 [SMILES: CC1(C)c2ccccc2-c2c(N(c3ccc(-c4ccccc4-c4ccccc4)cc3)c3ccc(-c4cc5ccccc5c5ccccc45)cc3)cccc21 ; CC1(C)c2ccccc2-c2c(N(c3ccc(-c4cc5ccccc5c5ccccc45)cc3)c3ccc(-c4ccccc4)c(-c4ccccc4)c3)cccc21 ; CC1(C)c2ccccc2-c2c(N(c3ccc(-c4cc5ccccc5c5ccccc45)cc3)c3cccc(-c4ccccc4)c3)cccc21] 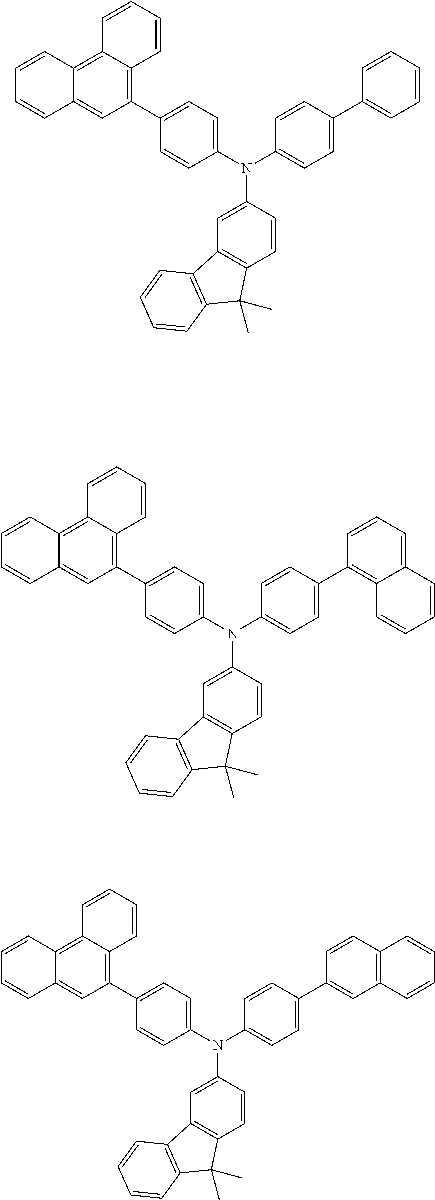 [SMILES: CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4cccc5ccccc45)cc3)c3ccc(-c4cc5ccccc5c5ccccc45)cc3)ccc21 ; CC1(C)c2ccccc2-c2cc(N(c3ccc(-c4ccc5ccccc5c4)cc3)c3ccc(-c4cc5ccccc5c5ccccc45)cc3)ccc21] 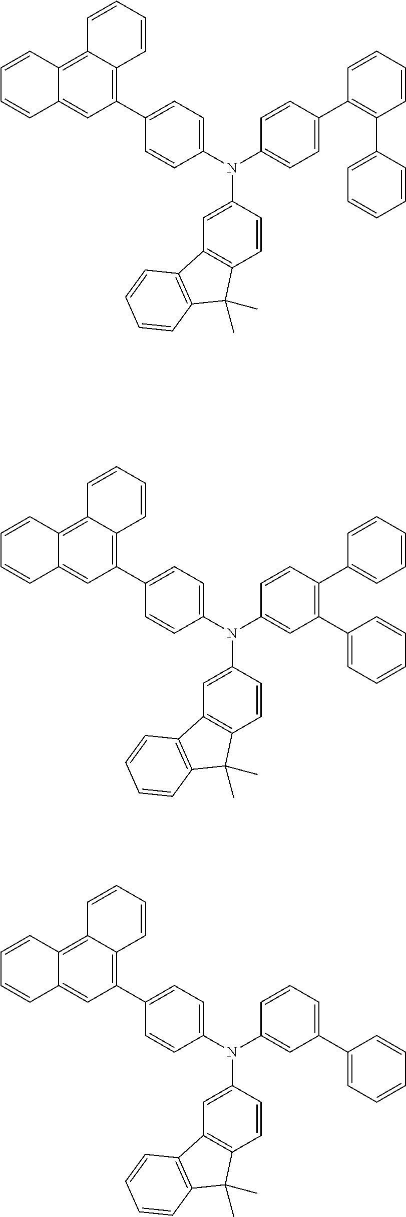 [SMILES: CC1(C)c2ccccc2-c2cc(N(c3ccc(-c4ccccc4-c4ccccc4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3ccc(-c4ccccc4)c(-c4ccccc4)c3)ccc21 ; CC1(C)c2ccccc2-c2cc(N(c3ccc(-c4cc5ccccc5c5ccccc45)cc3)c3cccc(-c4ccccc4)c3)ccc21] 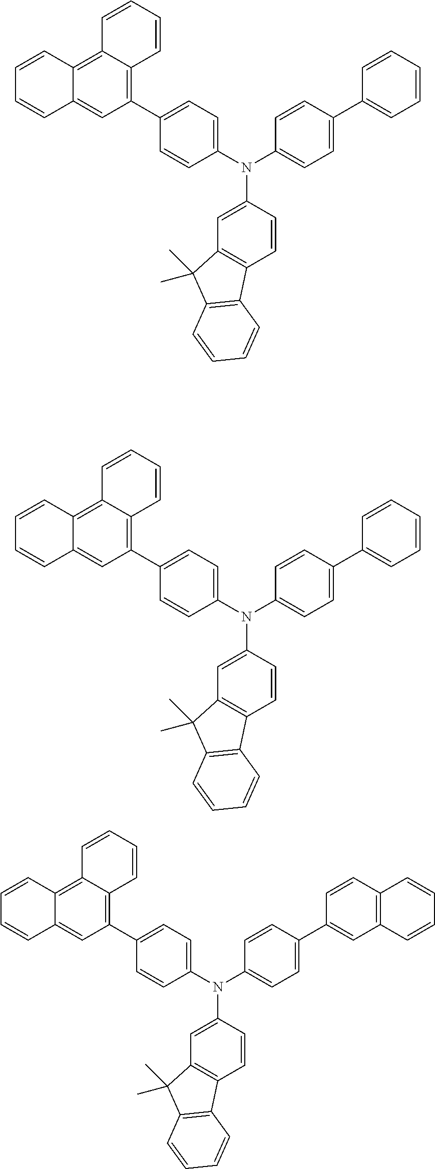 [SMILES: CC1(C)c2ccccc2-c2ccc(N(c3ccc(-c4ccccc4)cc3)c3ccc(-c4cc5ccccc5c5ccccc45)cc3)cc21 ; CC1(C)c2ccccc2-c2ccc(N(c3ccc(-c4ccccc4)cc3)c3ccc(-c4cc5ccccc5c5ccccc45)cc3)cc21 ; CC1(C)c2ccccc2-c2ccc(N(c3ccc(-c4ccc5ccccc5c4)cc3)c3ccc(-c4cc5ccccc5c5ccccc45)cc3)cc21] 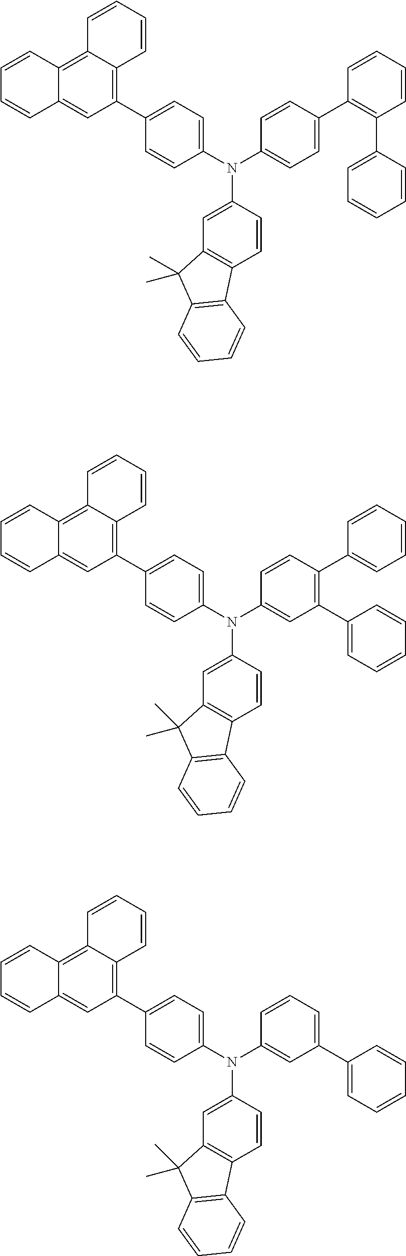 [SMILES: CC1(C)c2ccccc2-c2ccc(N(c3ccc(-c4ccccc4-c4ccccc4)cc3)c3ccc(-c4cc5ccccc5c5ccccc45)cc3)cc21 ; CC1(C)c2ccccc2-c2ccc(N(c3ccc(-c4cc5ccccc5c5ccccc45)cc3)c3ccc(-c4ccccc4)c(-c4ccccc4)c3)cc21 ; CC1(C)c2ccccc2-c2ccc(N(c3ccc(-c4cc5ccccc5c5ccccc45)cc3)c3cccc(-c4ccccc4)c3)cc21] 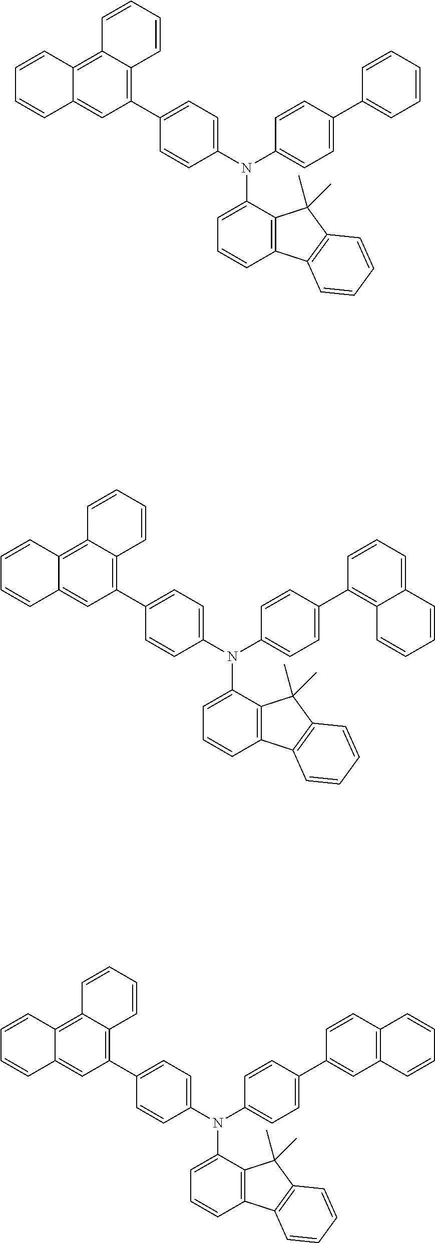 [SMILES: CC1(C)c2ccccc2-c2cccc(N(c3ccc(-c4ccccc4)cc3)c3ccc(-c4cc5ccccc5c5ccccc45)cc3)c21 ; CC1(C)c2ccccc2-c2cccc(N(c3ccc(-c4cccc5ccccc45)cc3)c3ccc(-c4cc5ccccc5c5ccccc45)cc3)c21 ; CC1(C)c2ccccc2-c2cccc(N(c3ccc(-c4ccc5ccccc5c4)cc3)c3ccc(-c4cc5ccccc5c5ccccc45)cc3)c21] 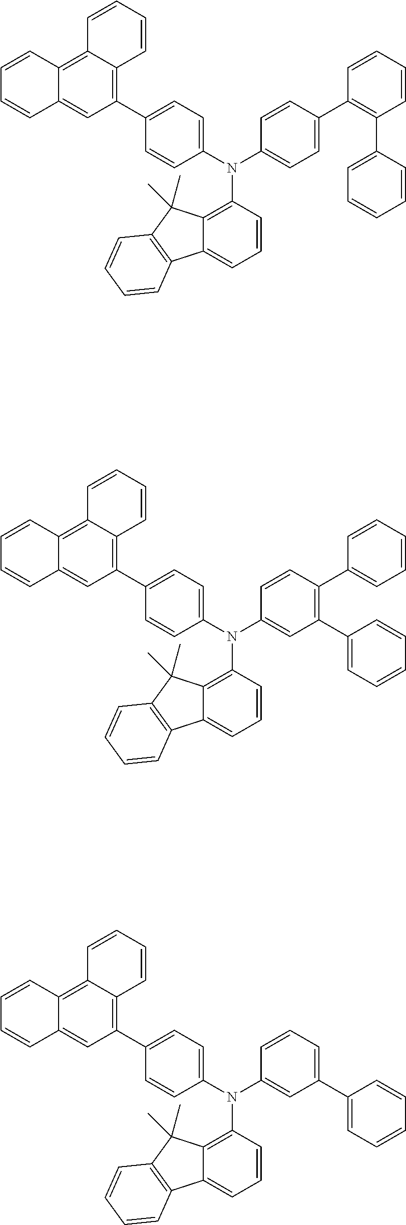 [SMILES: CC1(C)c2ccccc2-c2cccc(N(c3ccc(-c4ccccc4-c4ccccc4)cc3)c3ccc(-c4cc5ccccc5c5ccccc45)cc3)c21 ; CC1(C)c2ccccc2-c2cccc(N(c3ccc(-c4cc5ccccc5c5ccccc45)cc3)c3ccc(-c4ccccc4)c(-c4ccccc4)c3)c21 ; CC1(C)c2ccccc2-c2cccc(N(c3ccc(-c4cc5ccccc5c5ccccc45)cc3)c3cccc(-c4ccccc4)c3)c21] 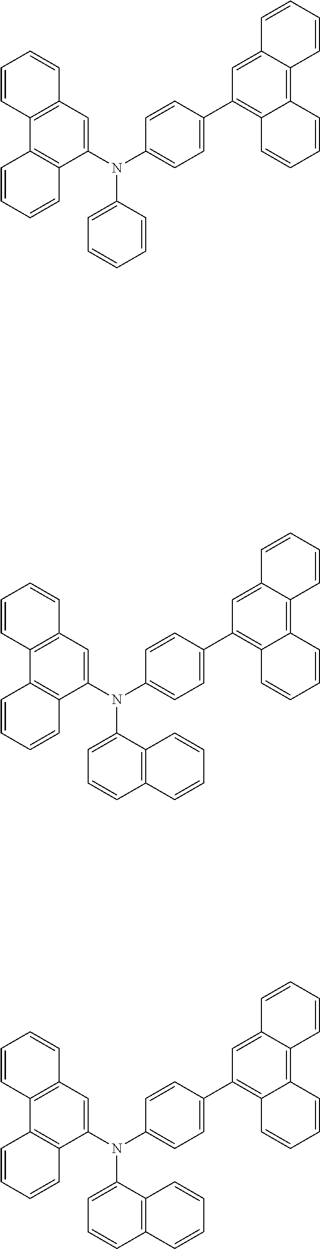 [SMILES: c1ccc(N(c2ccc(-c3cc4ccccc4c4ccccc34)cc2)c2cc3ccccc3c3ccccc23)cc1 ; c1ccc2c(N(c3ccc(-c4cc5ccccc5c5ccccc45)cc3)c3cc4ccccc4c4ccccc34)cccc2c1 ; c1ccc2c(N(c3ccc(-c4cc5ccccc5c5ccccc45)cc3)c3cc4ccccc4c4ccccc34)cccc2c1] 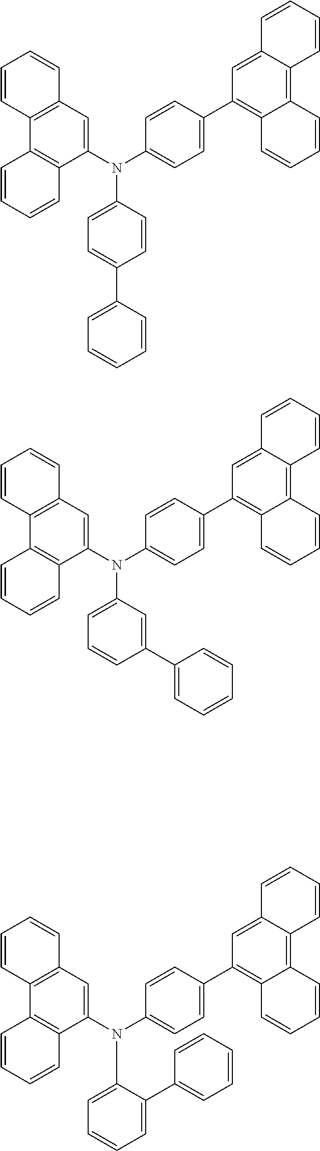 [SMILES: c1ccc(-c2ccc(N(c3ccc(-c4cc5ccccc5c5ccccc45)cc3)c3cc4ccccc4c4ccccc34)cc2)cc1 ; c1ccc(-c2cccc(N(c3ccc(-c4cc5ccccc5c5ccccc45)cc3)c3cc4ccccc4c4ccccc34)c2)cc1 ; c1ccc(-c2ccccc2N(c2ccc(-c3cc4ccccc4c4ccccc34)cc2)c2cc3ccccc3c3ccccc23)cc1] 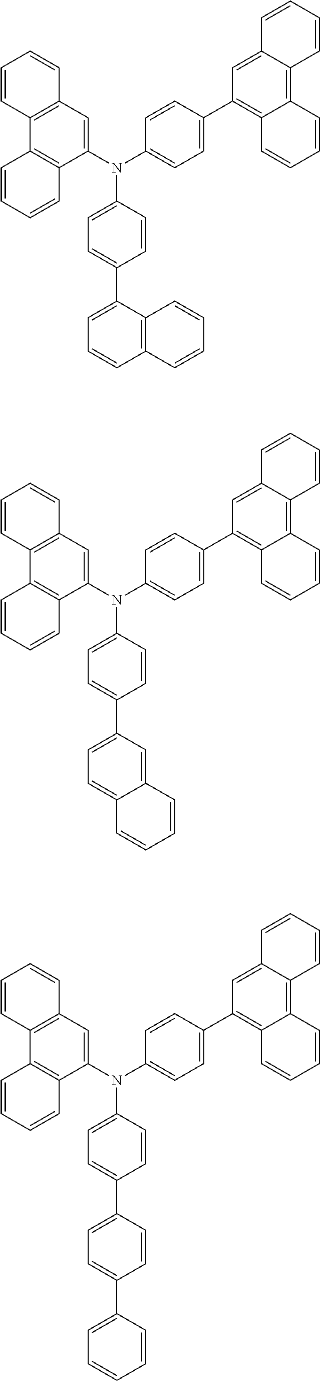 [SMILES: c1ccc2c(-c3ccc(N(c4ccc(-c5cc6ccccc6c6ccccc56)cc4)c4cc5ccccc5c5ccccc45)cc3)cccc2c1 ; c1ccc2cc(-c3ccc(N(c4ccc(-c5cc6ccccc6c6ccccc56)cc4)c4cc5ccccc5c5ccccc45)cc3)ccc2c1 ; c1ccc(-c2ccc(-c3ccc(N(c4ccc(-c5cc6ccccc6c6ccccc56)cc4)c4cc5ccccc5c5ccccc45)cc3)cc2)cc1] 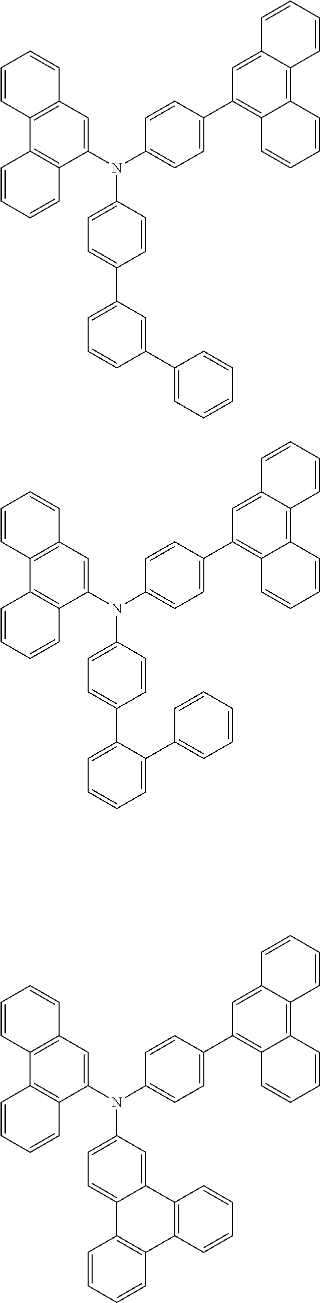 [SMILES: c1ccc(-c2cccc(-c3ccc(N(c4ccc(-c5cc6ccccc6c6ccccc56)cc4)c4cc5ccccc5c5ccccc45)cc3)c2)cc1 ; c1ccc(-c2ccccc2-c2ccc(N(c3ccc(-c4cc5ccccc5c5ccccc45)cc3)c3cc4ccccc4c4ccccc34)cc2)cc1 ; c1ccc2c(c1)cc(-c1ccc(N(c3ccc4c5ccccc5c5ccccc5c4c3)c3cc4ccccc4c4ccccc34)cc1)c1ccccc12] 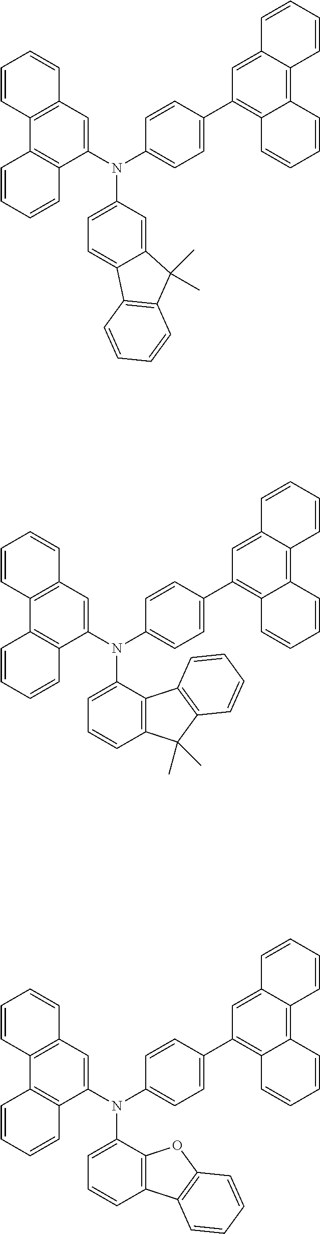 [SMILES: CC1(C)c2ccccc2-c2ccc(N(c3ccc(-c4cc5ccccc5c5ccccc45)cc3)c3cc4ccccc4c4ccccc34)cc21 ; CC1(C)c2ccccc2-c2c(N(c3ccc(-c4cc5ccccc5c5ccccc45)cc3)c3cc4ccccc4c4ccccc34)cccc21 ; c1ccc2c(c1)cc(-c1ccc(N(c3cc4ccccc4c4ccccc34)c3cccc4c3oc3ccccc34)cc1)c1ccccc12] 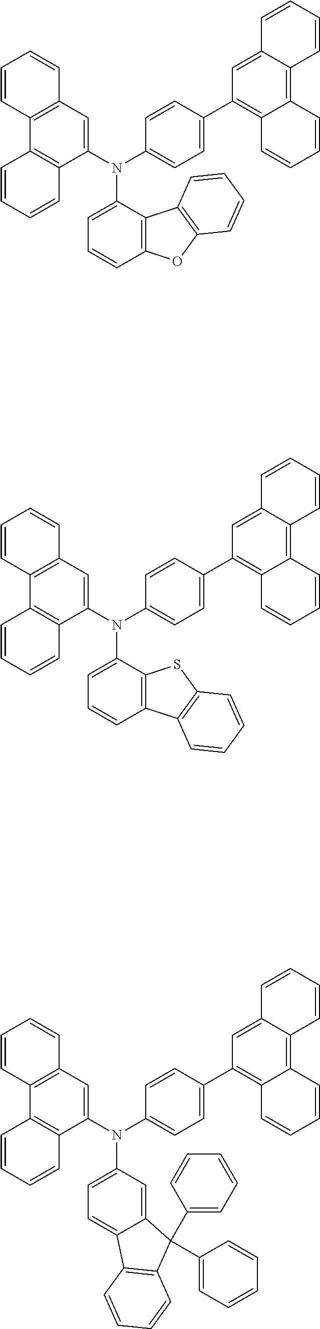 [SMILES: c1ccc2c(c1)cc(-c1ccc(N(c3cc4ccccc4c4ccccc34)c3cccc4oc5ccccc5c34)cc1)c1ccccc12 ; c1ccc2c(c1)cc(-c1ccc(N(c3cc4ccccc4c4ccccc34)c3cccc4c3sc3ccccc34)cc1)c1ccccc12 ; c1ccc(C2(c3ccccc3)c3ccccc3-c3ccc(N(c4ccc(-c5cc6ccccc6c6ccccc56)cc4)c4cc5ccccc5c5ccccc45)cc32)cc1] 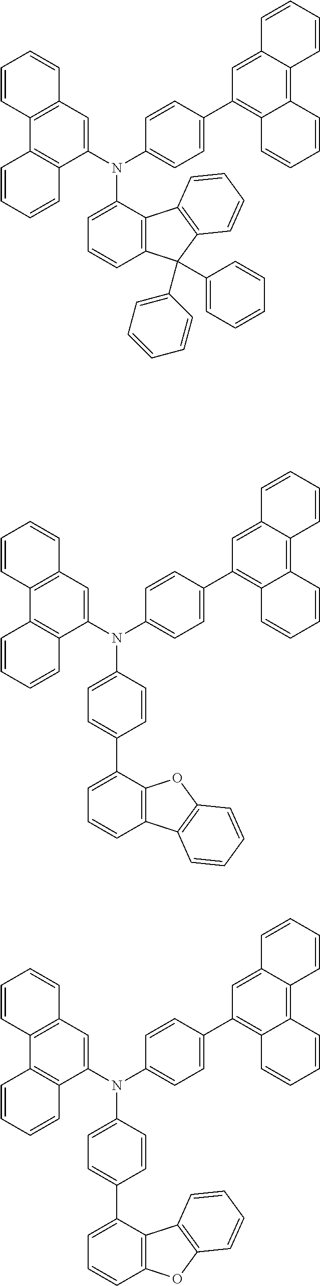 [SMILES: c1ccc(C2(c3ccccc3)c3ccccc3-c3c(N(c4ccc(-c5cc6ccccc6c6ccccc56)cc4)c4cc5ccccc5c5ccccc45)cccc32)cc1 ; c1ccc2c(c1)cc(-c1ccc(N(c3ccc(-c4cccc5c4oc4ccccc45)cc3)c3cc4ccccc4c4ccccc34)cc1)c1ccccc12 ; c1ccc2c(c1)cc(-c1ccc(N(c3ccc(-c4cccc5oc6ccccc6c45)cc3)c3cc4ccccc4c4ccccc34)cc1)c1ccccc12] 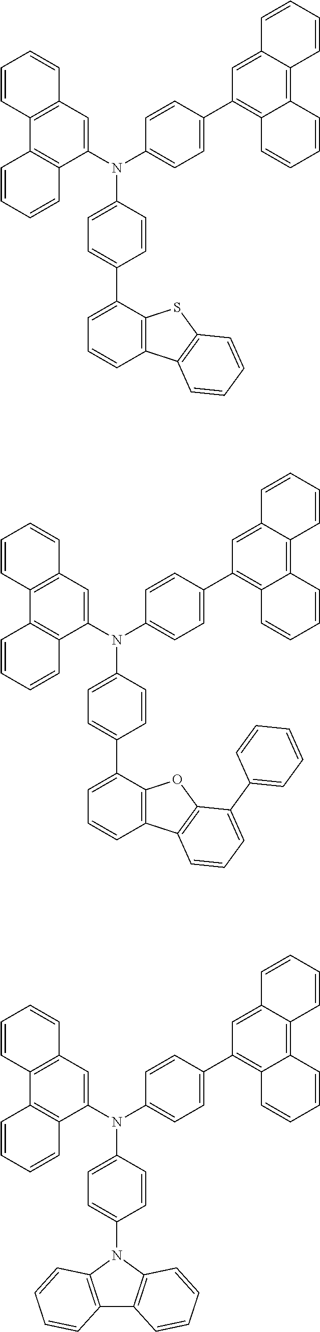 [SMILES: c1ccc2c(c1)cc(-c1ccc(N(c3ccc(-c4cccc5c4sc4ccccc45)cc3)c3cc4ccccc4c4ccccc34)cc1)c1ccccc12 ; c1ccc(-c2cccc3c2oc2c(-c4ccc(N(c5ccc(-c6cc7ccccc7c7ccccc67)cc5)c5cc6ccccc6c6ccccc56)cc4)cccc23)cc1 ; c1ccc2c(c1)cc(-c1ccc(N(c3ccc(-n4c5ccccc5c5ccccc54)cc3)c3cc4ccccc4c4ccccc34)cc1)c1ccccc12] 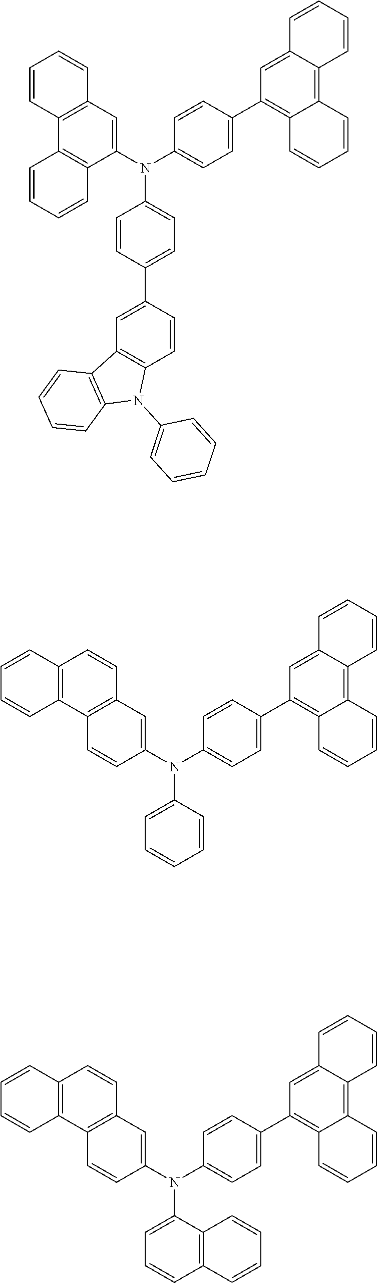 [SMILES: c1ccc(-n2c3ccccc3c3cc(-c4ccc(N(c5ccc(-c6cc7ccccc7c7ccccc67)cc5)c5cc6ccccc6c6ccccc56)cc4)ccc32)cc1 ; c1ccc(N(c2ccc(-c3cc4ccccc4c4ccccc34)cc2)c2ccc3c(ccc4ccccc43)c2)cc1 ; c1ccc2c(N(c3ccc(-c4cc5ccccc5c5ccccc45)cc3)c3ccc4c(ccc5ccccc54)c3)cccc2c1] 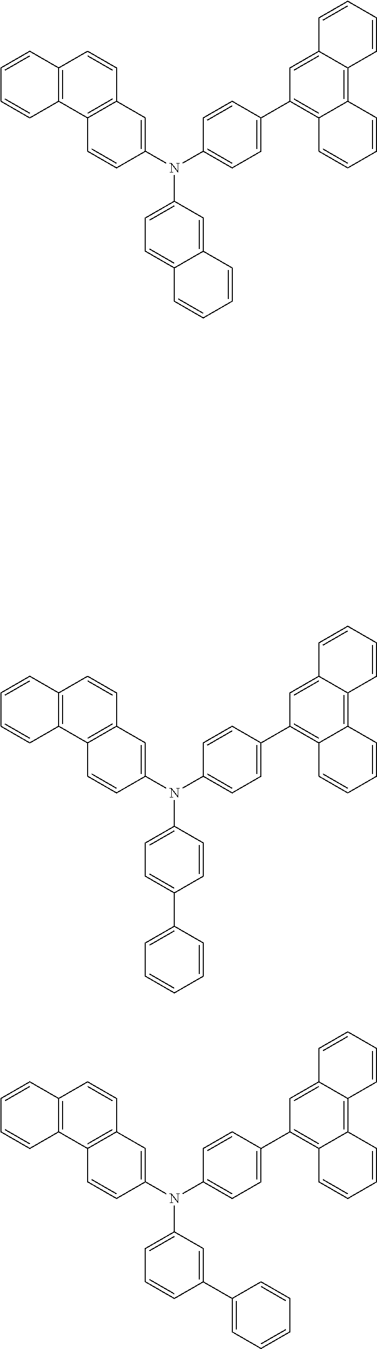 [SMILES: c1ccc2cc(N(c3ccc(-c4cc5ccccc5c5ccccc45)cc3)c3ccc4c(ccc5ccccc54)c3)ccc2c1 ; c1ccc(-c2ccc(N(c3ccc(-c4cc5ccccc5c5ccccc45)cc3)c3ccc4c(ccc5ccccc54)c3)cc2)cc1 ; c1ccc(-c2cccc(N(c3ccc(-c4cc5ccccc5c5ccccc45)cc3)c3ccc4c(ccc5ccccc54)c3)c2)cc1] 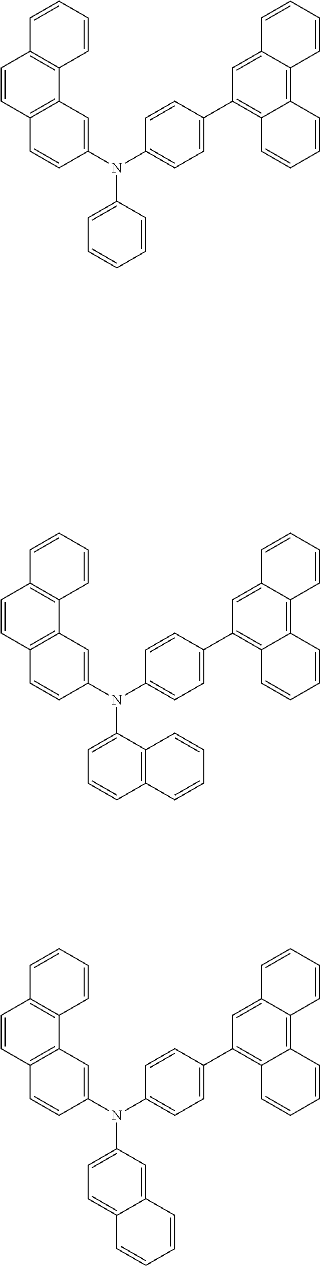 [SMILES: c1ccc(N(c2ccc(-c3cc4ccccc4c4ccccc34)cc2)c2ccc3ccc4ccccc4c3c2)cc1 ; c1ccc2c(N(c3ccc(-c4cc5ccccc5c5ccccc45)cc3)c3ccc4ccc5ccccc5c4c3)cccc2c1 ; c1ccc2cc(N(c3ccc(-c4cc5ccccc5c5ccccc45)cc3)c3ccc4ccc5ccccc5c4c3)ccc2c1] 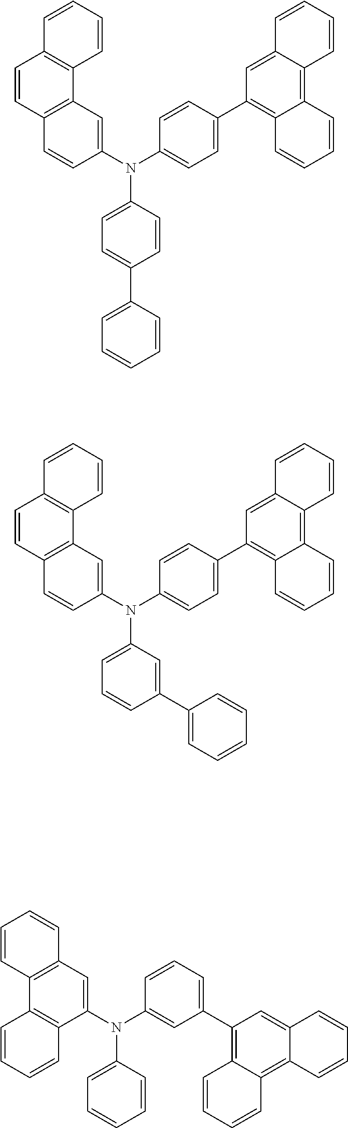 [SMILES: c1ccc(-c2ccc(N(c3ccc(-c4cc5ccccc5c5ccccc45)cc3)c3ccc4ccc5ccccc5c4c3)cc2)cc1 ; c1ccc(-c2cccc(N(c3ccc(-c4cc5ccccc5c5ccccc45)cc3)c3ccc4ccc5ccccc5c4c3)c2)cc1 ; c1ccc(N(c2cccc(-c3cc4ccccc4c4ccccc34)c2)c2cc3ccccc3c3ccccc23)cc1] 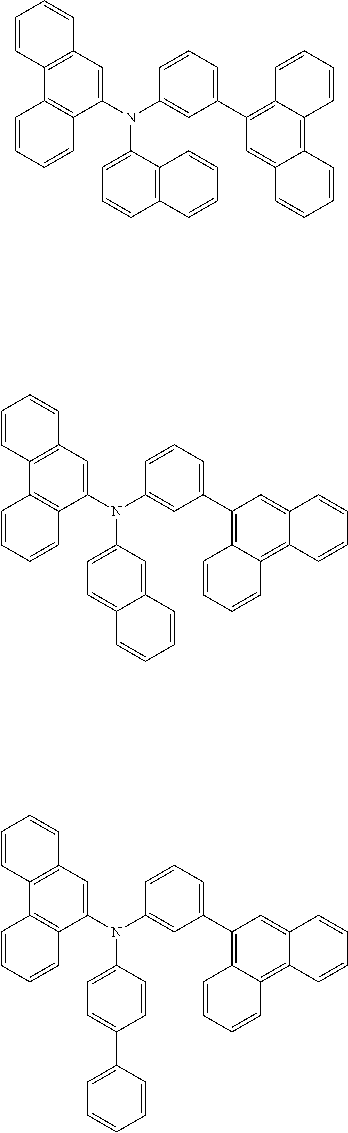 [SMILES: c1cc(-c2cc3ccccc3c3ccccc23)cc(N(c2cccc3ccccc23)c2cc3ccccc3c3ccccc23)c1 ; c1cc(-c2cc3ccccc3c3ccccc23)cc(N(c2ccc3ccccc3c2)c2cc3ccccc3c3ccccc23)c1 ; c1ccc(-c2ccc(N(c3cccc(-c4cc5ccccc5c5ccccc45)c3)c3cc4ccccc4c4ccccc34)cc2)cc1] 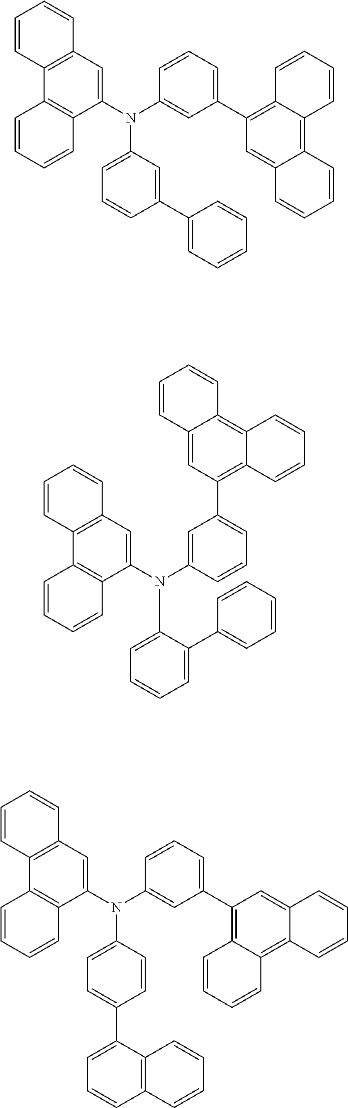 [SMILES: c1ccc(-c2cccc(N(c3cccc(-c4cc5ccccc5c5ccccc45)c3)c3cc4ccccc4c4ccccc34)c2)cc1 ; c1ccc(-c2ccccc2N(c2cccc(-c3cc4ccccc4c4ccccc34)c2)c2cc3ccccc3c3ccccc23)cc1 ; c1cc(-c2cc3ccccc3c3ccccc23)cc(N(c2ccc(-c3cccc4ccccc34)cc2)c2cc3ccccc3c3ccccc23)c1] 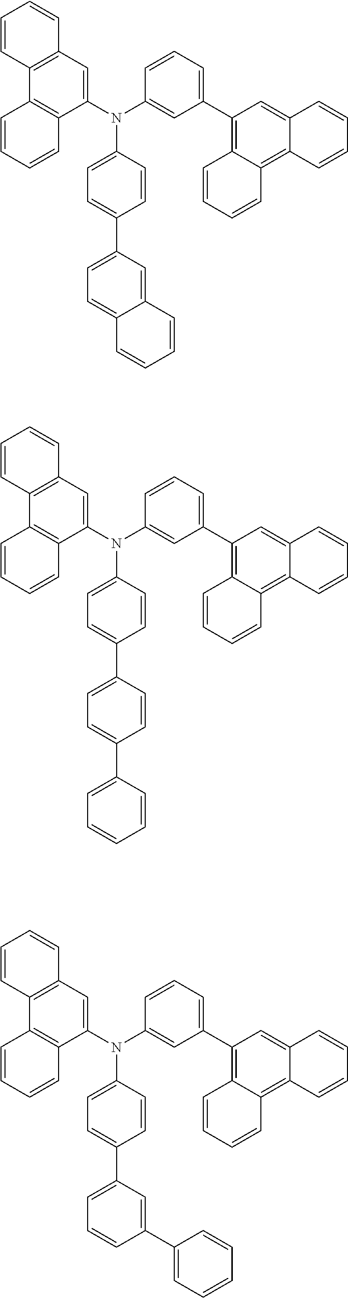 [SMILES: c1cc(-c2cc3ccccc3c3ccccc23)cc(N(c2ccc(-c3ccc4ccccc4c3)cc2)c2cc3ccccc3c3ccccc23)c1 ; c1ccc(-c2ccc(-c3ccc(N(c4cccc(-c5cc6ccccc6c6ccccc56)c4)c4cc5ccccc5c5ccccc45)cc3)cc2)cc1 ; c1ccc(-c2cccc(-c3ccc(N(c4cccc(-c5cc6ccccc6c6ccccc56)c4)c4cc5ccccc5c5ccccc45)cc3)c2)cc1] 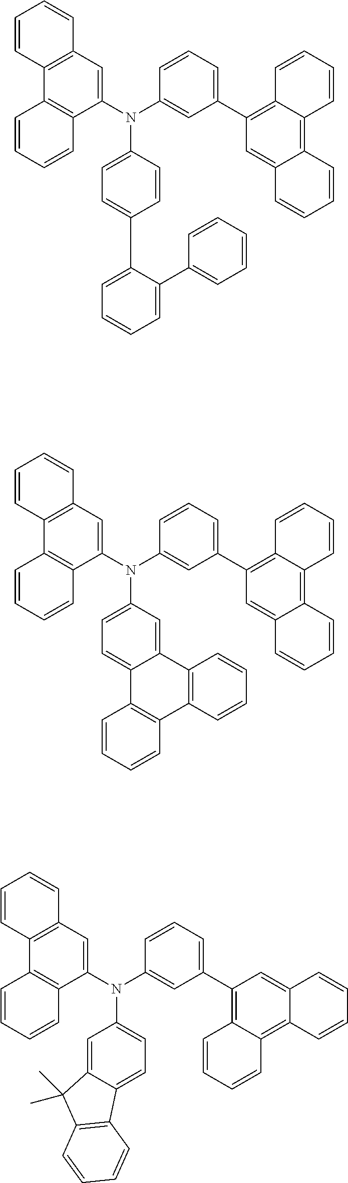 [SMILES: c1ccc(-c2ccccc2-c2ccc(N(c3cccc(-c4cc5ccccc5c5ccccc45)c3)c3cc4ccccc4c4ccccc34)cc2)cc1 ; c1cc(-c2cc3ccccc3c3ccccc23)cc(N(c2ccc3c4ccccc4c4ccccc4c3c2)c2cc3ccccc3c3ccccc23)c1 ; CC1(C)c2ccccc2-c2ccc(N(c3cccc(-c4cc5ccccc5c5ccccc45)c3)c3cc4ccccc4c4ccccc34)cc21] 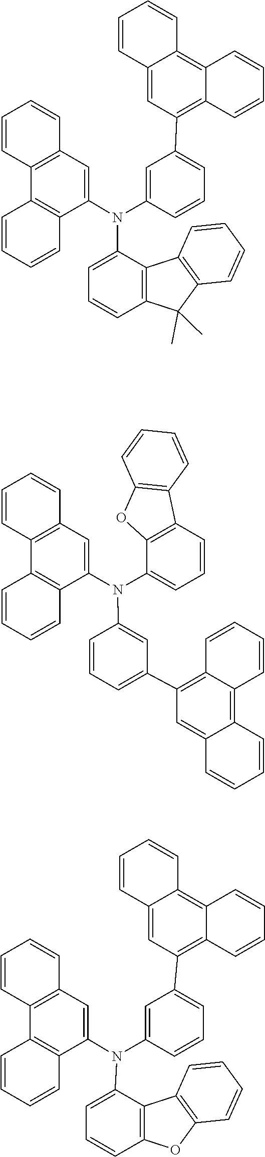 [SMILES: CC1(C)c2ccccc2-c2c(N(c3cccc(-c4cc5ccccc5c5ccccc45)c3)c3cc4ccccc4c4ccccc34)cccc21 ; c1cc(-c2cc3ccccc3c3ccccc23)cc(N(c2cc3ccccc3c3ccccc23)c2cccc3c2oc2ccccc23)c1 ; c1cc(-c2cc3ccccc3c3ccccc23)cc(N(c2cc3ccccc3c3ccccc23)c2cccc3oc4ccccc4c23)c1] 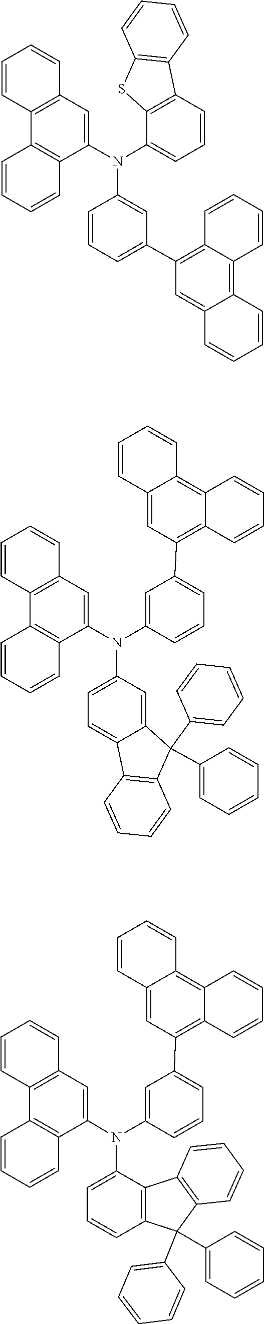 [SMILES: c1cc(-c2cc3ccccc3c3ccccc23)cc(N(c2cc3ccccc3c3ccccc23)c2cccc3c2sc2ccccc23)c1 ; c1ccc(C2(c3ccccc3)c3ccccc3-c3ccc(N(c4cccc(-c5cc6ccccc6c6ccccc56)c4)c4cc5ccccc5c5ccccc45)cc32)cc1 ; c1ccc(C2(c3ccccc3)c3ccccc3-c3c(N(c4cccc(-c5cc6ccccc6c6ccccc56)c4)c4cc5ccccc5c5ccccc45)cccc32)cc1] 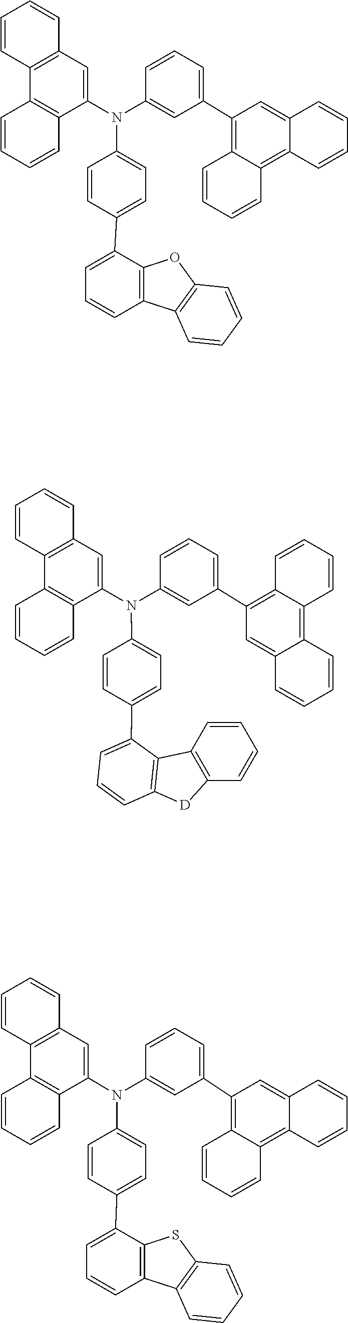 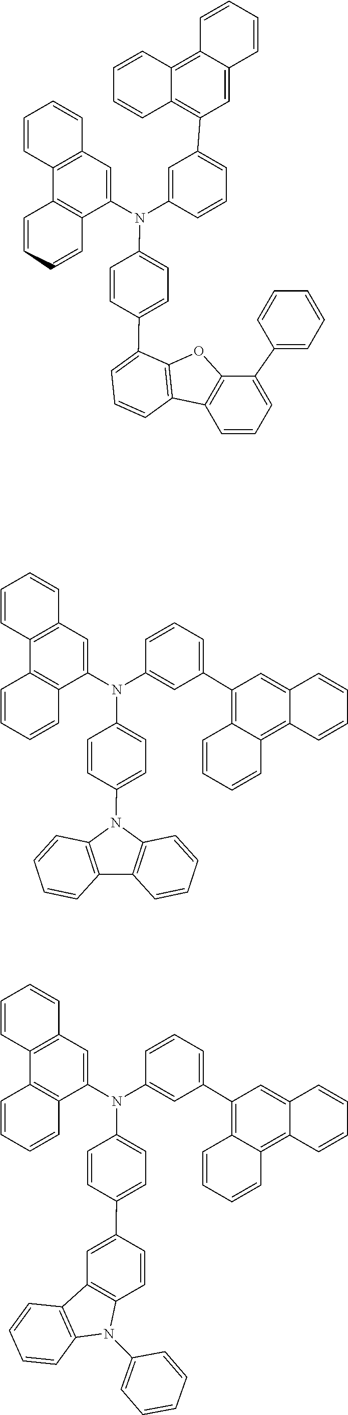 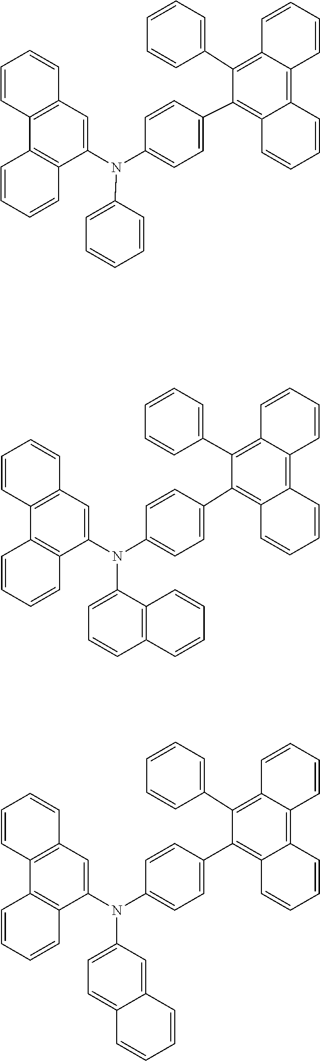 [SMILES: c1ccc(-c2c(-c3ccc(N(c4ccccc4)c4cc5ccccc5c5ccccc45)cc3)c3ccccc3c3ccccc23)cc1 ; c1ccc(-c2c(-c3ccc(N(c4cccc5ccccc45)c4cc5ccccc5c5ccccc45)cc3)c3ccccc3c3ccccc23)cc1 ; c1ccc(-c2c(-c3ccc(N(c4ccc5ccccc5c4)c4cc5ccccc5c5ccccc45)cc3)c3ccccc3c3ccccc23)cc1] 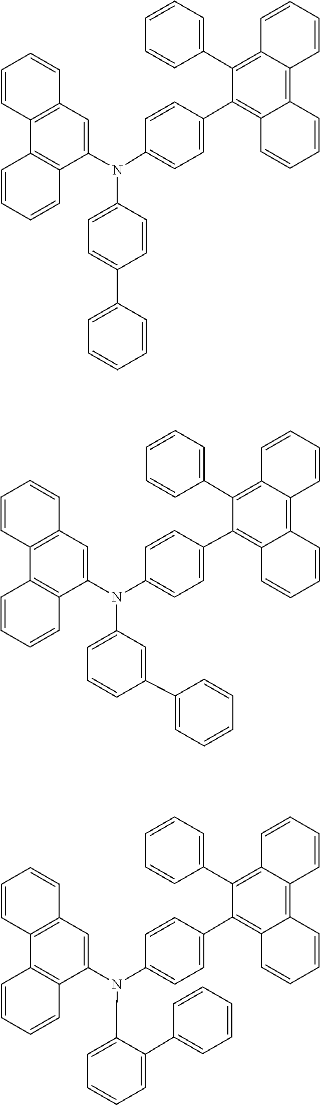 [SMILES: c1ccc(-c2ccc(N(c3ccc(-c4c(-c5ccccc5)c5ccccc5c5ccccc45)cc3)c3cc4ccccc4c4ccccc34)cc2)cc1 ; c1ccc(-c2cccc(N(c3ccc(-c4c(-c5ccccc5)c5ccccc5c5ccccc45)cc3)c3cc4ccccc4c4ccccc34)c2)cc1 ; c1ccc(-c2ccccc2N(c2ccc(-c3c(-c4ccccc4)c4ccccc4c4ccccc34)cc2)c2cc3ccccc3c3ccccc23)cc1] 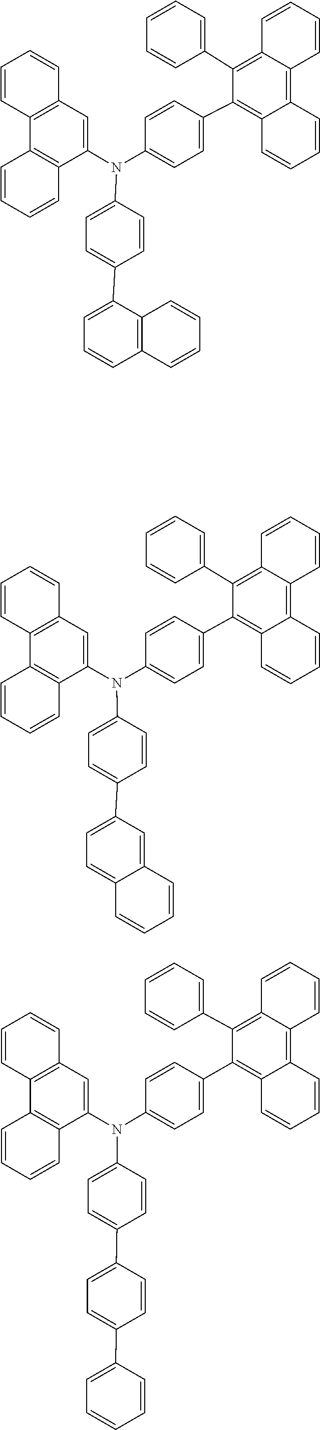 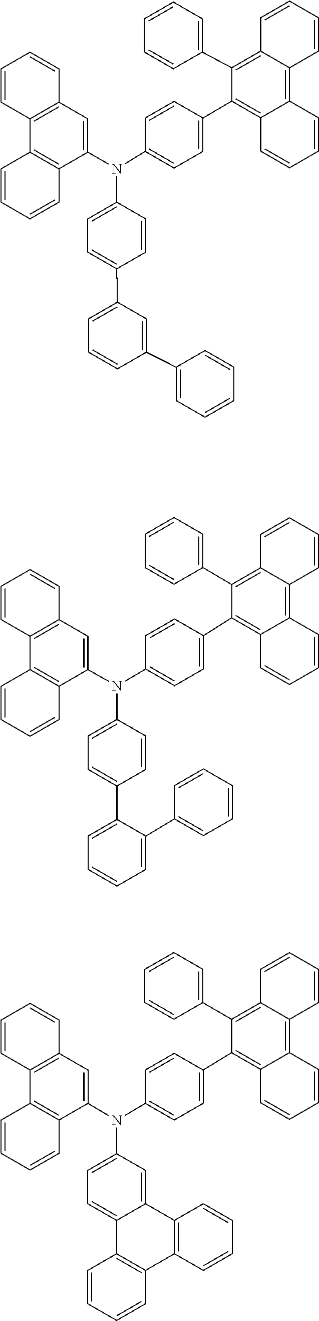 [SMILES: c1ccc(-c2cccc(-c3ccc(N(c4ccc(-c5c(-c6ccccc6)c6ccccc6c6ccccc56)cc4)c4cc5ccccc5c5ccccc45)cc3)c2)cc1 ; c1ccc(-c2ccccc2-c2ccc(N(c3ccc(-c4c(-c5ccccc5)c5ccccc5c5ccccc45)cc3)c3cc4ccccc4c4ccccc34)cc2)cc1 ; c1ccc(-c2c(-c3ccc(N(c4ccc5c6ccccc6c6ccccc6c5c4)c4cc5ccccc5c5ccccc45)cc3)c3ccccc3c3ccccc23)cc1] 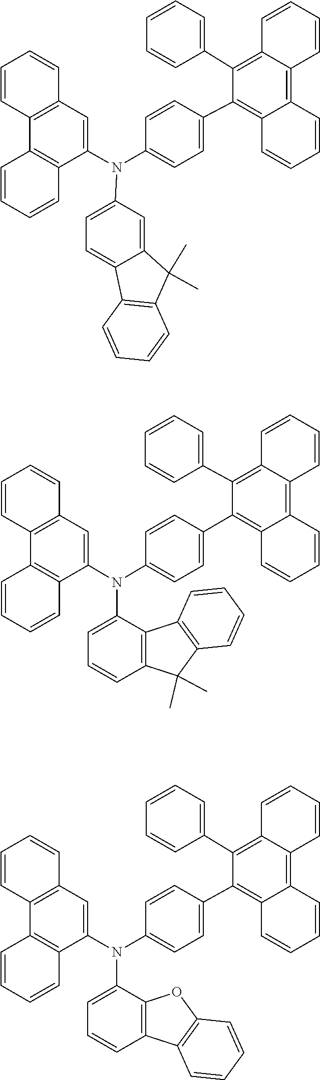 [SMILES: CC1(C)c2ccccc2-c2ccc(N(c3ccc(-c4c(-c5ccccc5)c5ccccc5c5ccccc45)cc3)c3cc4ccccc4c4ccccc34)cc21 ; CC1(C)c2ccccc2-c2c(N(c3ccc(-c4c(-c5ccccc5)c5ccccc5c5ccccc45)cc3)c3cc4ccccc4c4ccccc34)cccc21 ; c1ccc(-c2c(-c3ccc(N(c4cc5ccccc5c5ccccc45)c4cccc5c4oc4ccccc45)cc3)c3ccccc3c3ccccc23)cc1] 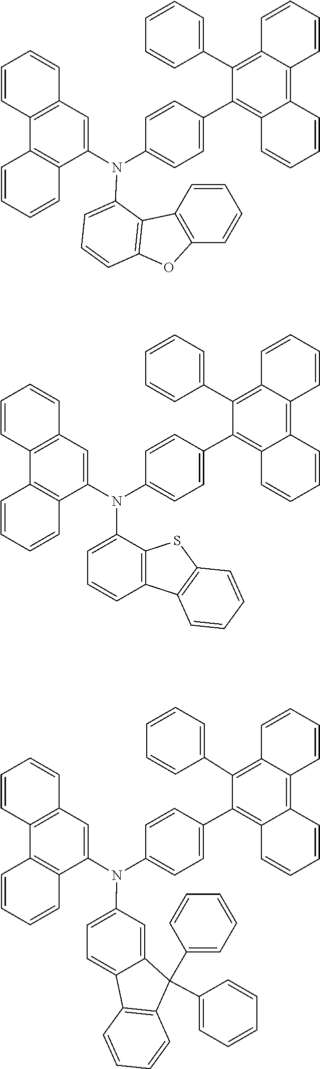 [SMILES: c1ccc(-c2c(-c3ccc(N(c4cc5ccccc5c5ccccc45)c4cccc5oc6ccccc6c45)cc3)c3ccccc3c3ccccc23)cc1 ; c1ccc(-c2c(-c3ccc(N(c4cc5ccccc5c5ccccc45)c4cccc5c4sc4ccccc45)cc3)c3ccccc3c3ccccc23)cc1 ; c1ccc(-c2c(-c3ccc(N(c4ccc5c(c4)C(c4ccccc4)(c4ccccc4)c4ccccc4-5)c4cc5ccccc5c5ccccc45)cc3)c3ccccc3c3ccccc23)cc1] 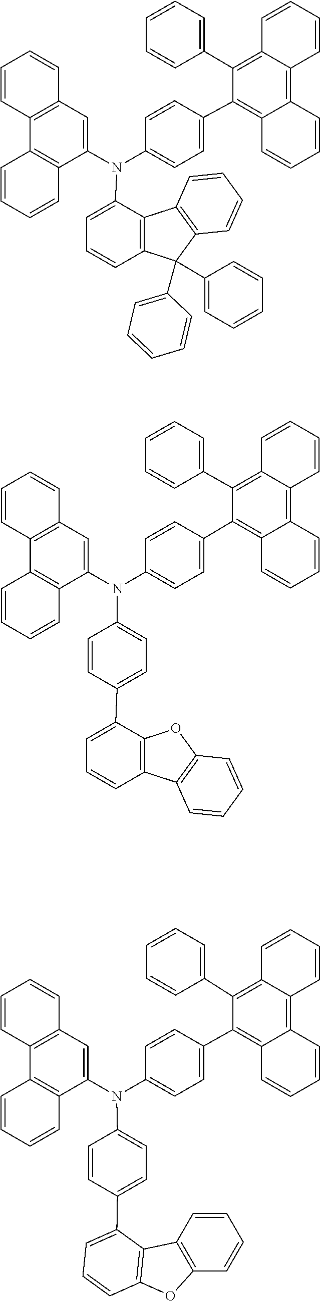 [SMILES: c1ccc(-c2c(-c3ccc(N(c4cccc5c4-c4ccccc4C5(c4ccccc4)c4ccccc4)c4cc5ccccc5c5ccccc45)cc3)c3ccccc3c3ccccc23)cc1 ; c1ccc(-c2c(-c3ccc(N(c4ccc(-c5cccc6c5oc5ccccc56)cc4)c4cc5ccccc5c5ccccc45)cc3)c3ccccc3c3ccccc23)cc1 ; c1ccc(-c2c(-c3ccc(N(c4ccc(-c5cccc6oc7ccccc7c56)cc4)c4cc5ccccc5c5ccccc45)cc3)c3ccccc3c3ccccc23)cc1] 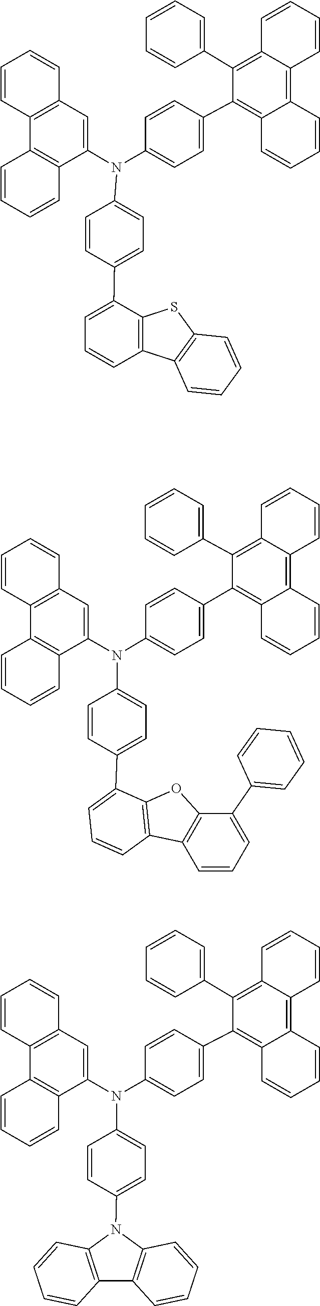 [SMILES: c1ccc(-c2c(-c3ccc(N(c4ccc(-c5cccc6c5sc5ccccc56)cc4)c4cc5ccccc5c5ccccc45)cc3)c3ccccc3c3ccccc23)cc1 ; c1ccc(-c2c(-c3ccc(N(c4ccc(-c5cccc6c5oc5c(-c7ccccc7)cccc56)cc4)c4cc5ccccc5c5ccccc45)cc3)c3ccccc3c3ccccc23)cc1 ; c1ccc(-c2c(-c3ccc(N(c4ccc(-n5c6ccccc6c6ccccc65)cc4)c4cc5ccccc5c5ccccc45)cc3)c3ccccc3c3ccccc23)cc1] 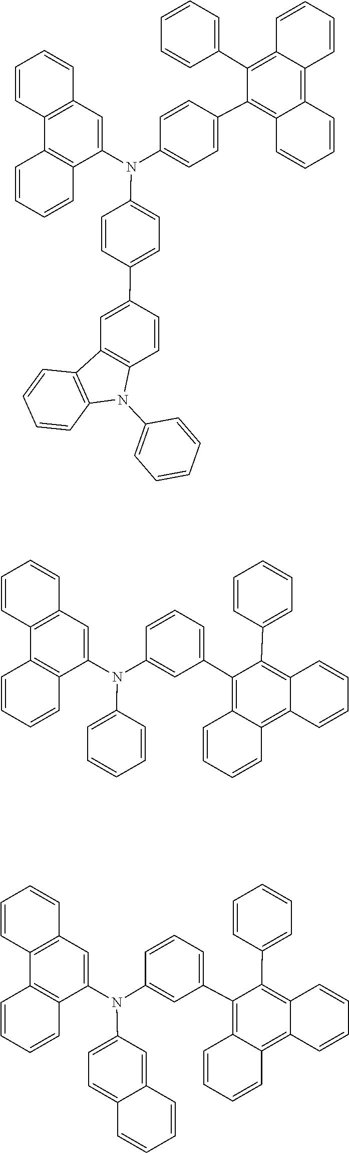 [SMILES: c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1 ; c1ccc(-c2c(-c3cccc(N(c4ccccc4)c4cc5ccccc5c5ccccc45)c3)c3ccccc3c3ccccc23)cc1 ; c1ccc(-c2c(-c3cccc(N(c4ccc5ccccc5c4)c4cc5ccccc5c5ccccc45)c3)c3ccccc3c3ccccc23)cc1] 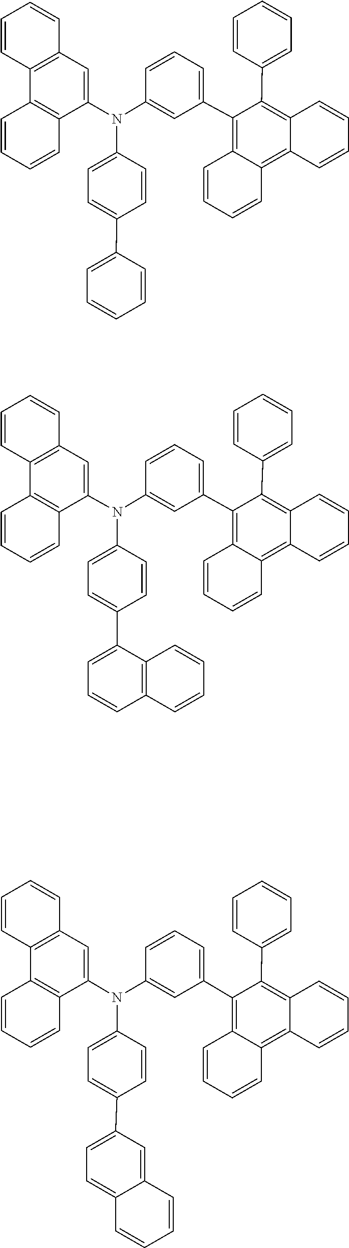 [SMILES: c1ccc(-c2ccc(N(c3cccc(-c4c(-c5ccccc5)c5ccccc5c5ccccc45)c3)c3cc4ccccc4c4ccccc34)cc2)cc1 ; c1ccc(-c2c(-c3cccc(N(c4ccc(-c5cccc6ccccc56)cc4)c4cc5ccccc5c5ccccc45)c3)c3ccccc3c3ccccc23)cc1 ; c1ccc(-c2c(-c3cccc(N(c4ccc(-c5ccc6ccccc6c5)cc4)c4cc5ccccc5c5ccccc45)c3)c3ccccc3c3ccccc23)cc1] 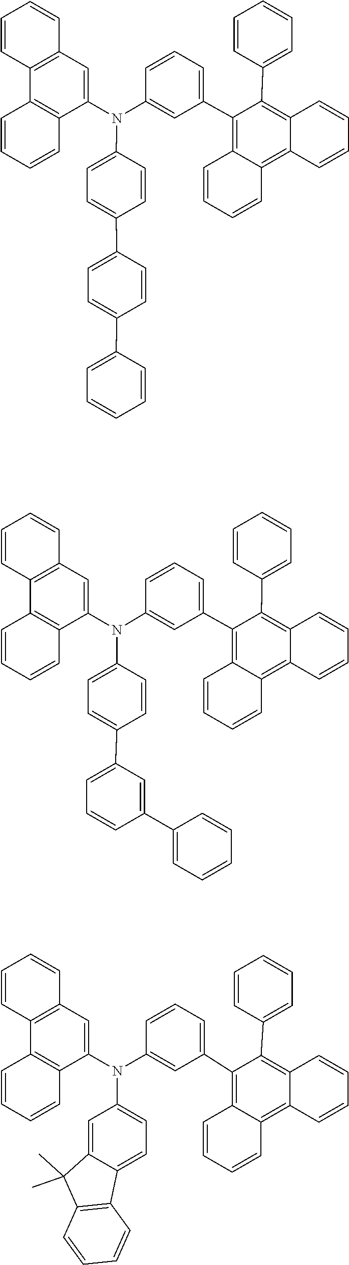 [SMILES: c1ccc(-c2ccc(-c3ccc(N(c4cccc(-c5c(-c6ccccc6)c6ccccc6c6ccccc56)c4)c4cc5ccccc5c5ccccc45)cc3)cc2)cc1 ; c1ccc(-c2cccc(-c3ccc(N(c4cccc(-c5c(-c6ccccc6)c6ccccc6c6ccccc56)c4)c4cc5ccccc5c5ccccc45)cc3)c2)cc1 ; CC1(C)c2ccccc2-c2ccc(N(c3cccc(-c4c(-c5ccccc5)c5ccccc5c5ccccc45)c3)c3cc4ccccc4c4ccccc34)cc21] 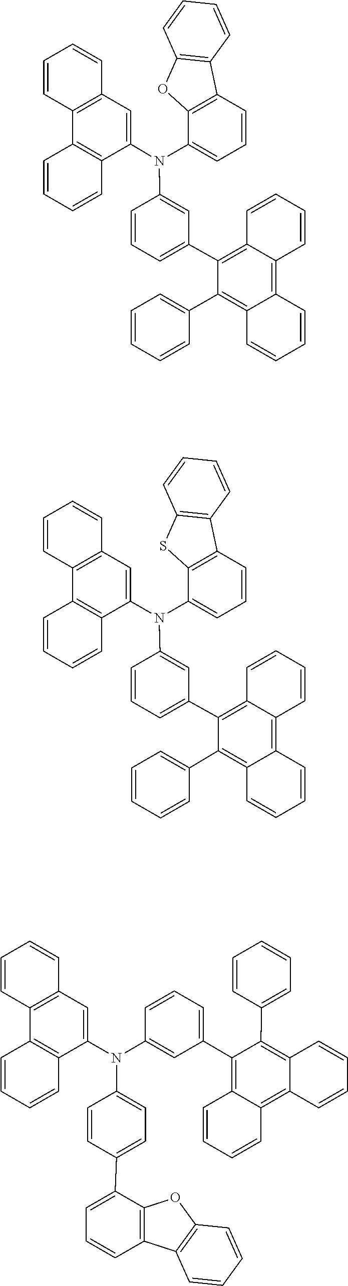 [SMILES: c1ccc(-c2c(-c3cccc(N(c4cc5ccccc5c5ccccc45)c4cccc5c4oc4ccccc45)c3)c3ccccc3c3ccccc23)cc1 ; c1ccc(-c2c(-c3cccc(N(c4cc5ccccc5c5ccccc45)c4cccc5c4sc4ccccc45)c3)c3ccccc3c3ccccc23)cc1 ; c1ccc(-c2c(-c3cccc(N(c4ccc(-c5cccc6c5oc5ccccc56)cc4)c4cc5ccccc5c5ccccc45)c3)c3ccccc3c3ccccc23)cc1] 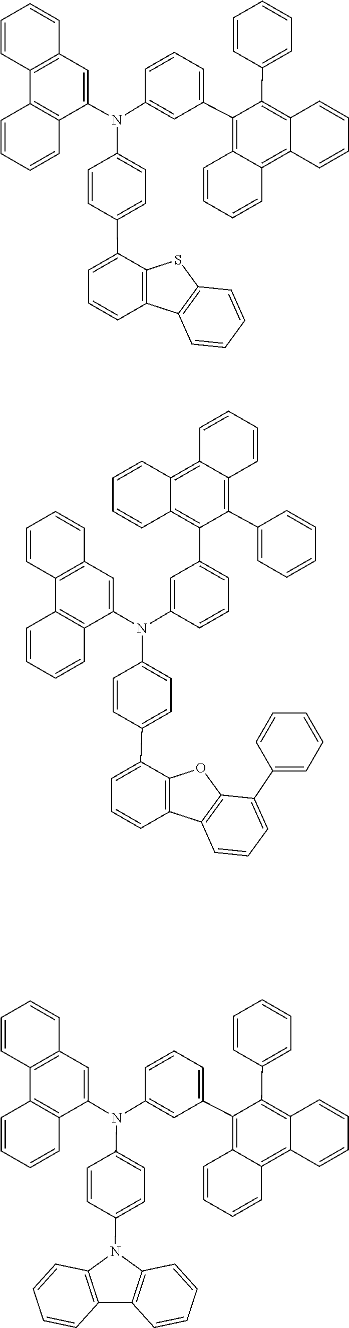 [SMILES: c1ccc(-c2c(-c3cccc(N(c4ccc(-c5cccc6c5sc5ccccc56)cc4)c4cc5ccccc5c5ccccc45)c3)c3ccccc3c3ccccc23)cc1 ; c1ccc(-c2c(-c3cccc(N(c4ccc(-c5cccc6c5oc5c(-c7ccccc7)cccc56)cc4)c4cc5ccccc5c5ccccc45)c3)c3ccccc3c3ccccc23)cc1 ; c1ccc(-c2c(-c3cccc(N(c4ccc(-n5c6ccccc6c6ccccc65)cc4)c4cc5ccccc5c5ccccc45)c3)c3ccccc3c3ccccc23)cc1] 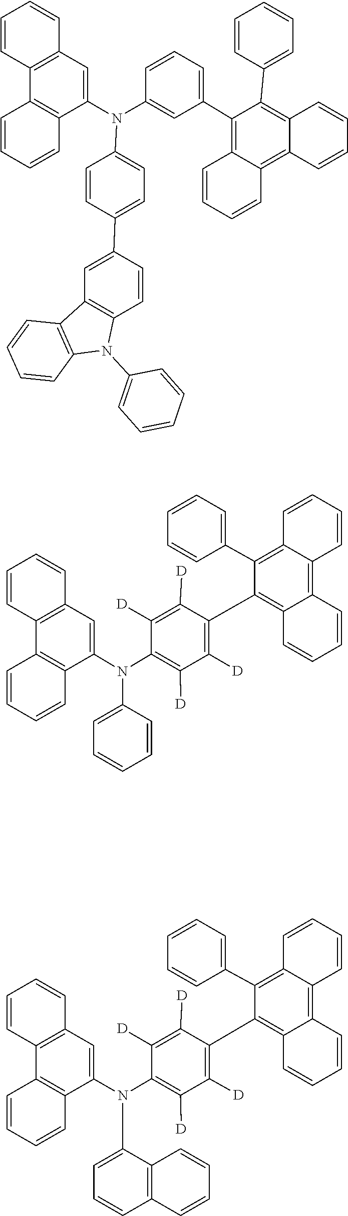 [SMILES: c1ccc(-c2c(-c3cccc(N(c4ccc(-c5ccc6c(c5)c5ccccc5n6-c5ccccc5)cc4)c4cc5ccccc5c5ccccc45)c3)c3ccccc3c3ccccc23)cc1 ; [2H]c1c([2H])c(N(c2ccccc2)c2cc3ccccc3c3ccccc23)c([2H])c([2H])c1-c1c(-c2ccccc2)c2ccccc2c2ccccc12 ; [2H]c1c([2H])c(N(c2cccc3ccccc23)c2cc3ccccc3c3ccccc23)c([2H])c([2H])c1-c1c(-c2ccccc2)c2ccccc2c2ccccc12] 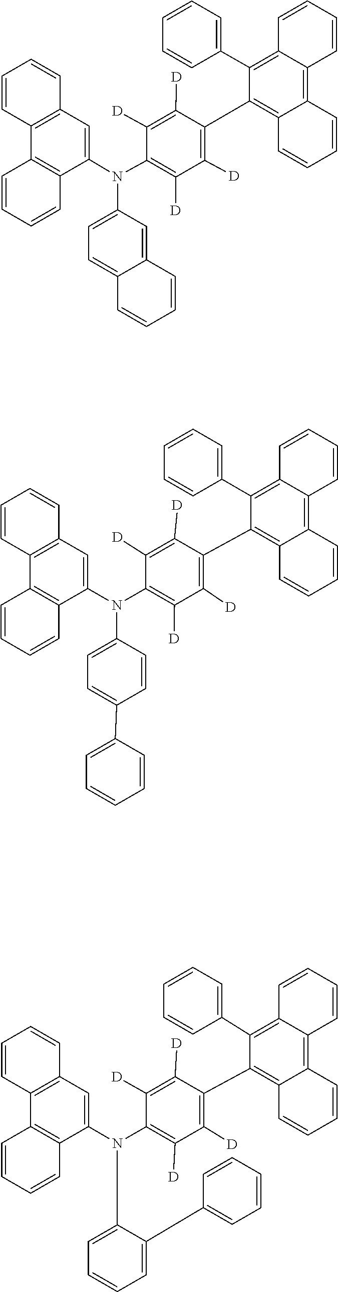 [SMILES: [2H]c1c([2H])c(N(c2ccc3ccccc3c2)c2cc3ccccc3c3ccccc23)c([2H])c([2H])c1-c1c(-c2ccccc2)c2ccccc2c2ccccc12 ; [2H]c1c([2H])c(N(c2ccc(-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccccc2-c2ccccc2)c2cc3ccccc3c3ccccc23)c([2H])c([2H])c1-c1c(-c2ccccc2)c2ccccc2c2ccccc12] 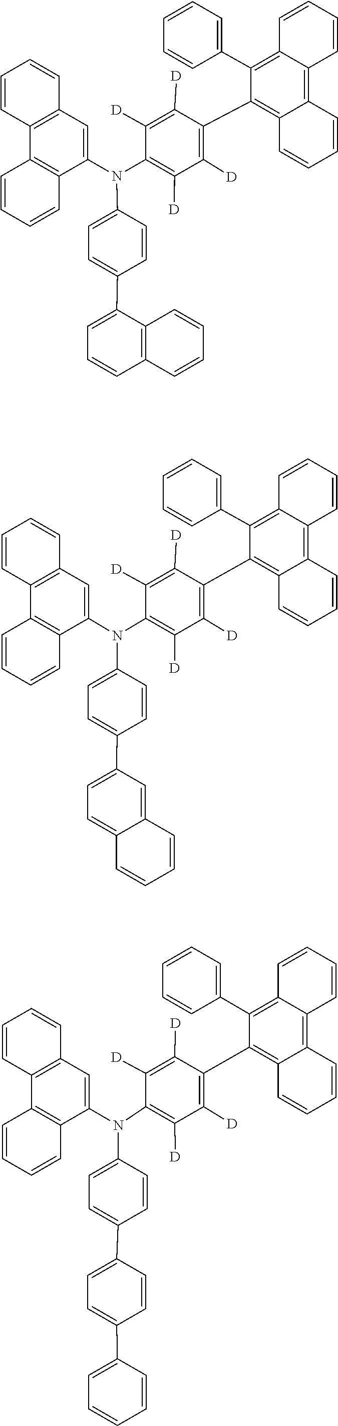 [SMILES: [2H]c1c([2H])c(N(c2ccc(-c3cccc4ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc4ccccc4c3)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1c(-c2ccccc2)c2ccccc2c2ccccc12] 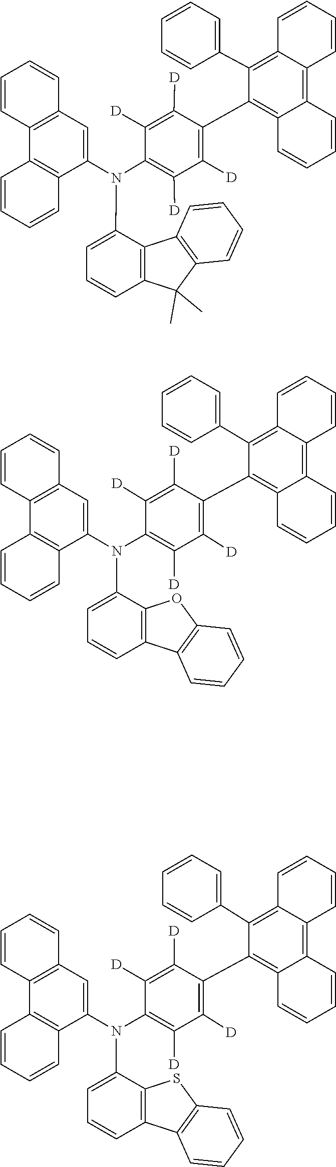 [SMILES: [2H]c1c([2H])c(N(c2cccc3c2-c2ccccc2C3(C)C)c2cc3ccccc3c3ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2oc2ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2sc2ccccc23)c([2H])c([2H])c1-c1c(-c2ccccc2)c2ccccc2c2ccccc12] 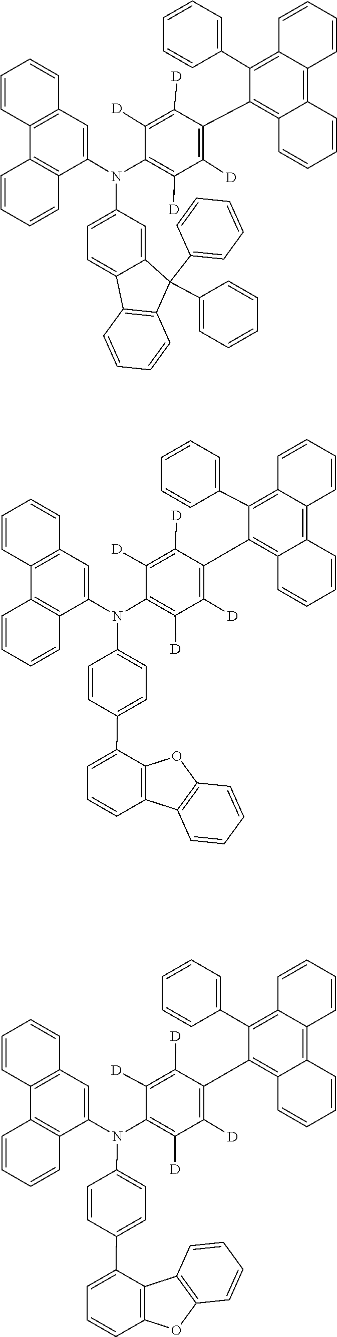 [SMILES: [2H]c1c([2H])c(N(c2ccc3c(c2)C(c2ccccc2)(c2ccccc2)c2ccccc2-3)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1c(-c2ccccc2)c2ccccc2c2ccccc12] 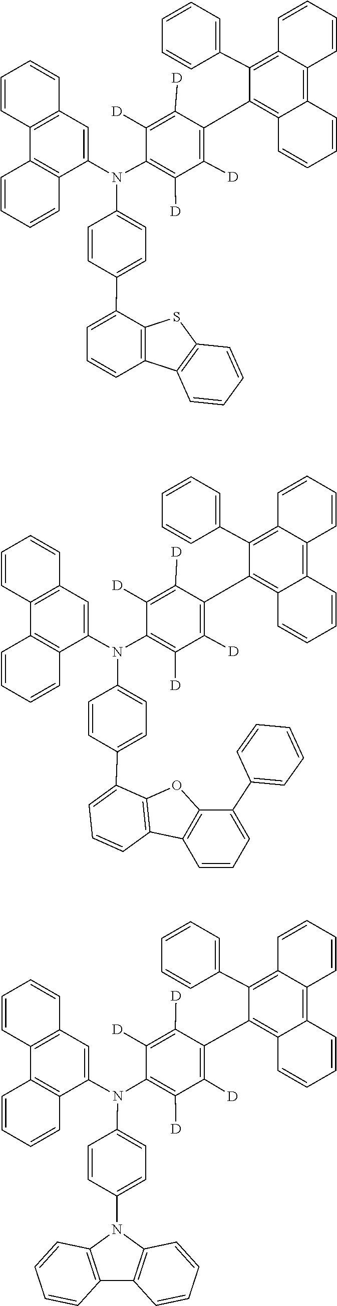 [SMILES: [2H]c1c([2H])c(N(c2ccc(-c3cccc4c3sc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1c(-c2ccccc2)c2ccccc2c2ccccc12] 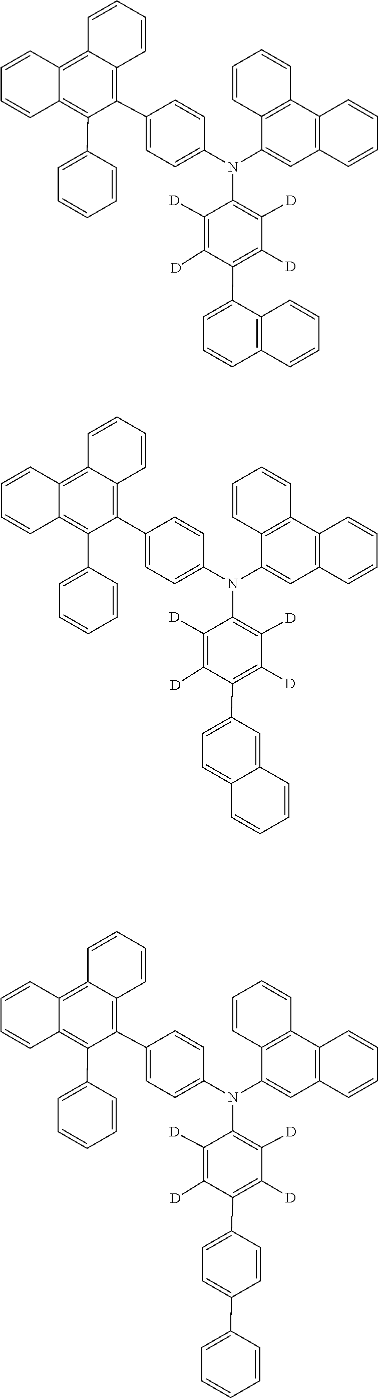 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc2ccccc2c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc(-c2ccccc2)cc1] 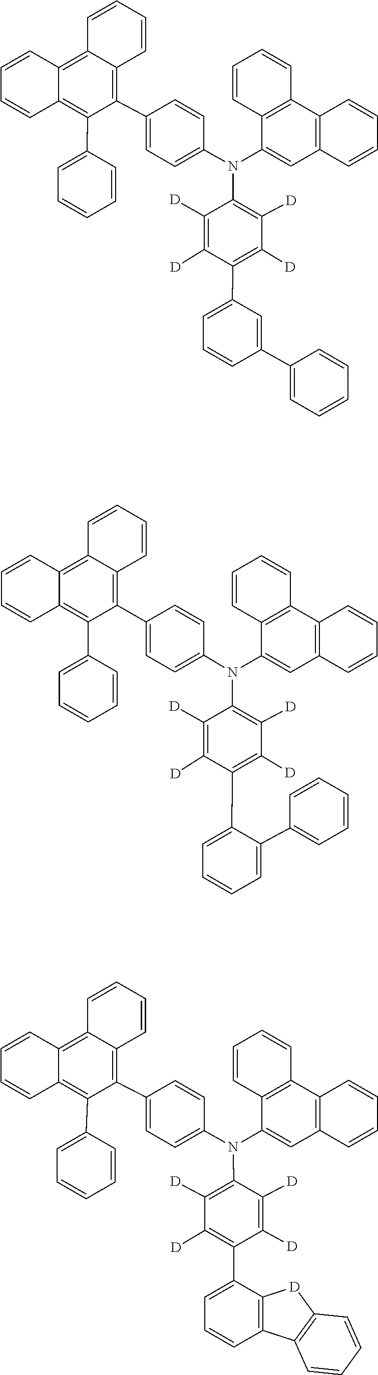 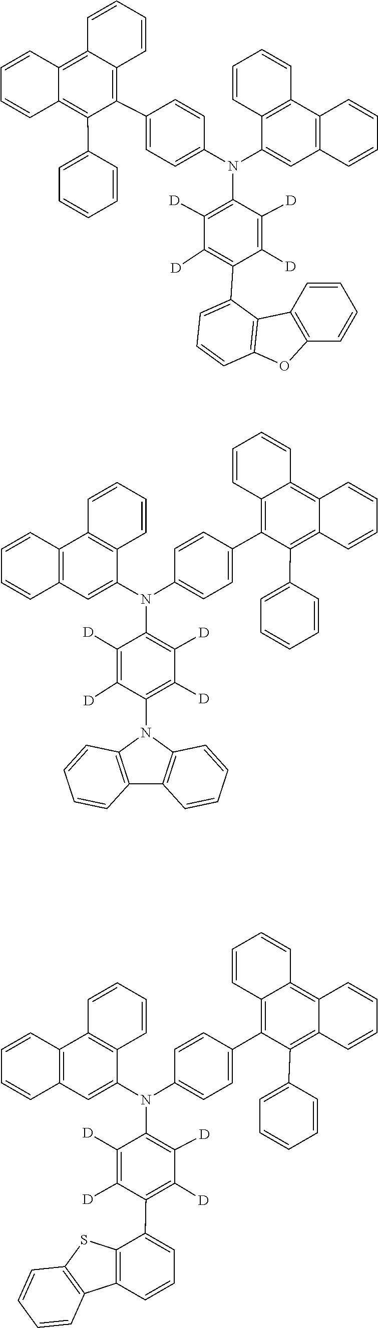 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2oc3ccccc3c12 ; [2H]c1c([2H])c(-n2c3ccccc3c3ccccc32)c([2H])c([2H])c1N(c1ccc(-c2c(-c3ccccc3)c3ccccc3c3ccccc23)cc1)c1cc2ccccc2c2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1sc1ccccc12] 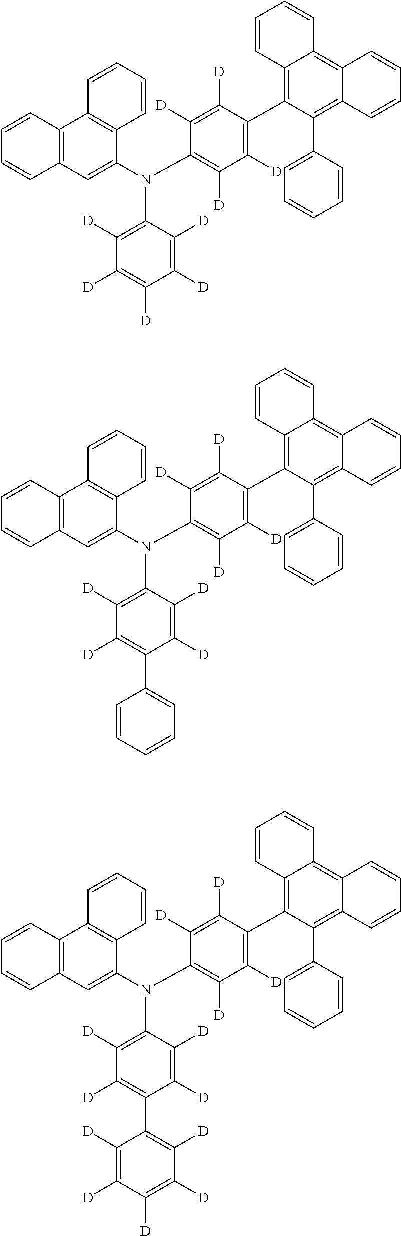 [SMILES: [2H]c1c([2H])c([2H])c(N(c2c([2H])c([2H])c(-c3c(-c4ccccc4)c4ccccc4c4ccccc34)c([2H])c2[2H])c2cc3ccccc3c3ccccc23)c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4c(-c5ccccc5)c5ccccc5c5ccccc45)c([2H])c3[2H])c3cc4ccccc4c4ccccc34)c([2H])c2[2H])c([2H])c1[2H]] 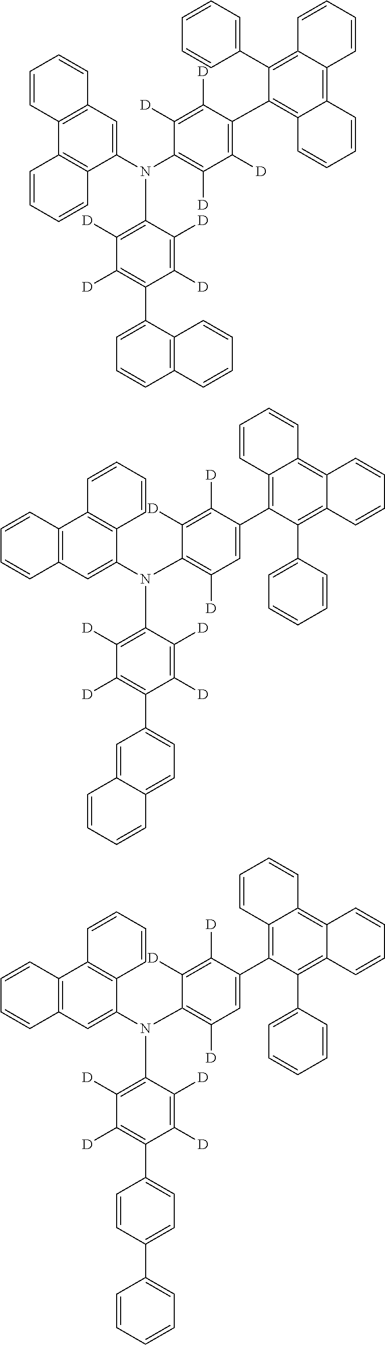 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1cccc2ccccc12 ; [2H]c1cc(-c2c(-c3ccccc3)c3ccccc3c3ccccc23)c([2H])c([2H])c1N(c1c([2H])c([2H])c(-c2ccc3ccccc3c2)c([2H])c1[2H])c1cc2ccccc2c2ccccc12 ; [2H]c1cc(-c2c(-c3ccccc3)c3ccccc3c3ccccc23)c([2H])c([2H])c1N(c1c([2H])c([2H])c(-c2ccc(-c3ccccc3)cc2)c([2H])c1[2H])c1cc2ccccc2c2ccccc12] 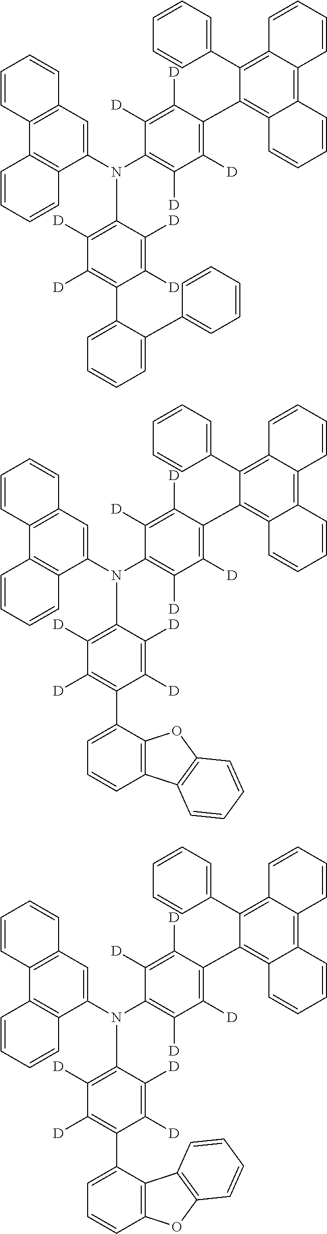 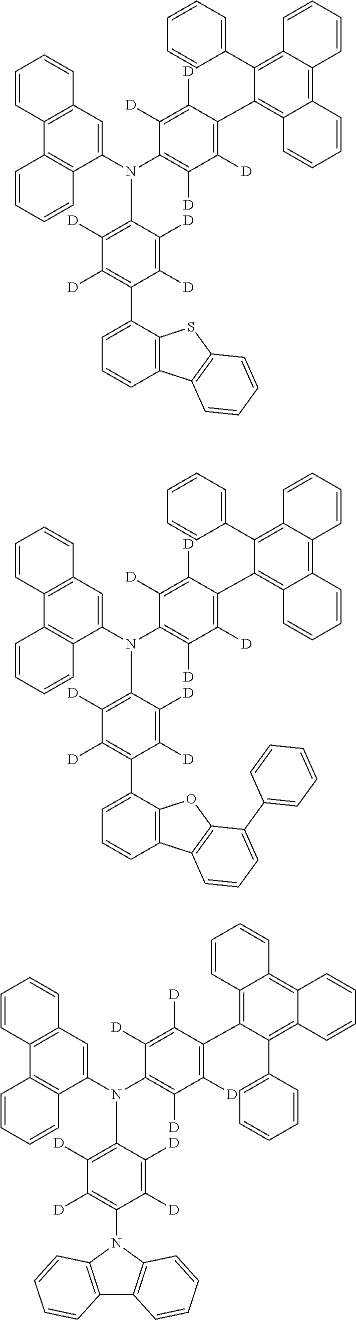 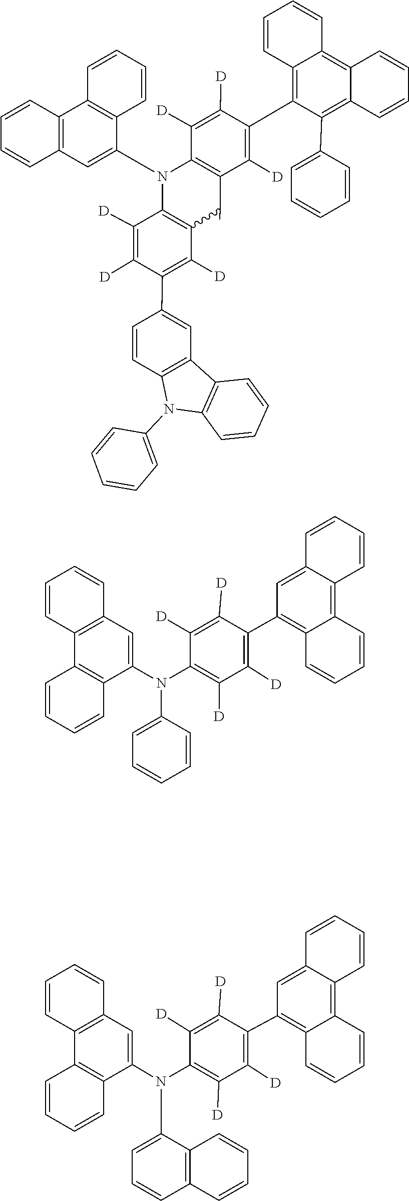 [SMILES: [2H]c1c([2H])c2c(c([2H])c1-c1ccc3c(c1)c1ccccc1n3-c1ccccc1)C(C)c1c([2H])c(-c3c(-c4ccccc4)c4ccccc4c4ccccc34)c([2H])c([2H])c1N2c1cc2ccccc2c2ccccc12 ; [2H]c1c([2H])c(N(c2ccccc2)c2cc3ccccc3c3ccccc23)c([2H])c([2H])c1-c1cc2ccccc2c2ccccc12 ; [2H]c1c([2H])c(N(c2cccc3ccccc23)c2cc3ccccc3c3ccccc23)c([2H])c([2H])c1-c1cc2ccccc2c2ccccc12] 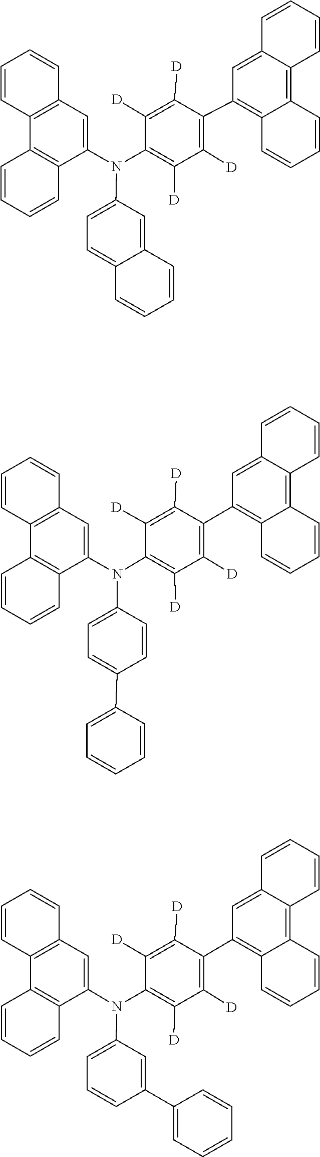 [SMILES: [2H]c1c([2H])c(N(c2ccc3ccccc3c2)c2cc3ccccc3c3ccccc23)c([2H])c([2H])c1-c1cc2ccccc2c2ccccc12 ; [2H]c1c([2H])c(N(c2ccc(-c3ccccc3)cc2)c2cc3ccccc3c3ccccc23)c([2H])c([2H])c1-c1cc2ccccc2c2ccccc12 ; [2H]c1c([2H])c(N(c2cccc(-c3ccccc3)c2)c2cc3ccccc3c3ccccc23)c([2H])c([2H])c1-c1cc2ccccc2c2ccccc12] 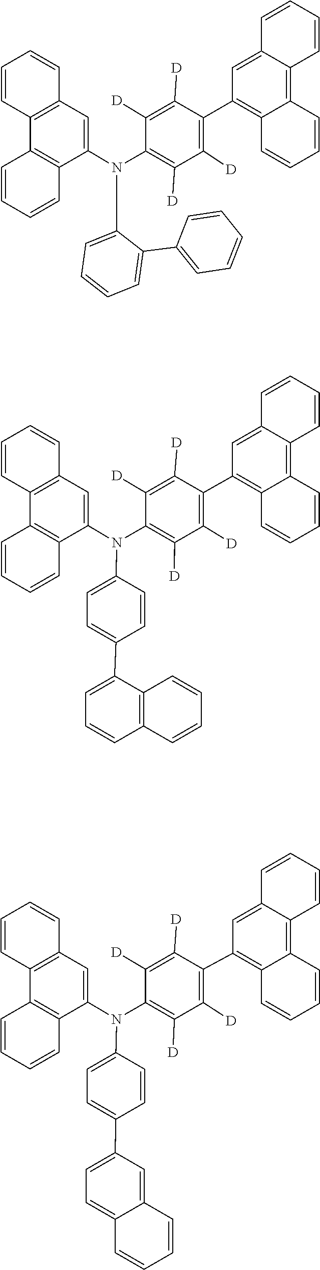 [SMILES: [2H]c1c([2H])c(N(c2ccccc2-c2ccccc2)c2cc3ccccc3c3ccccc23)c([2H])c([2H])c1-c1cc2ccccc2c2ccccc12 ; [2H]c1c([2H])c(N(c2ccc(-c3cccc4ccccc34)cc2)c2cc3ccccc3c3ccccc23)c([2H])c([2H])c1-c1cc2ccccc2c2ccccc12 ; [2H]c1c([2H])c(N(c2ccc(-c3ccc4ccccc4c3)cc2)c2cc3ccccc3c3ccccc23)c([2H])c([2H])c1-c1cc2ccccc2c2ccccc12] 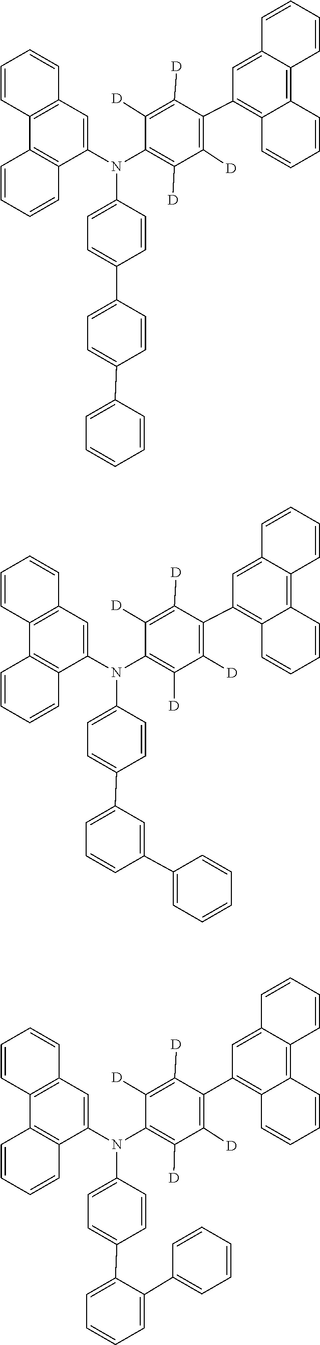 [SMILES: [2H]c1c([2H])c(N(c2ccc(-c3ccc(-c4ccccc4)cc3)cc2)c2cc3ccccc3c3ccccc23)c([2H])c([2H])c1-c1cc2ccccc2c2ccccc12 ; [2H]c1c([2H])c(N(c2ccc(-c3cccc(-c4ccccc4)c3)cc2)c2cc3ccccc3c3ccccc23)c([2H])c([2H])c1-c1cc2ccccc2c2ccccc12 ; [2H]c1c([2H])c(N(c2ccc(-c3ccccc3-c3ccccc3)cc2)c2cc3ccccc3c3ccccc23)c([2H])c([2H])c1-c1cc2ccccc2c2ccccc12] 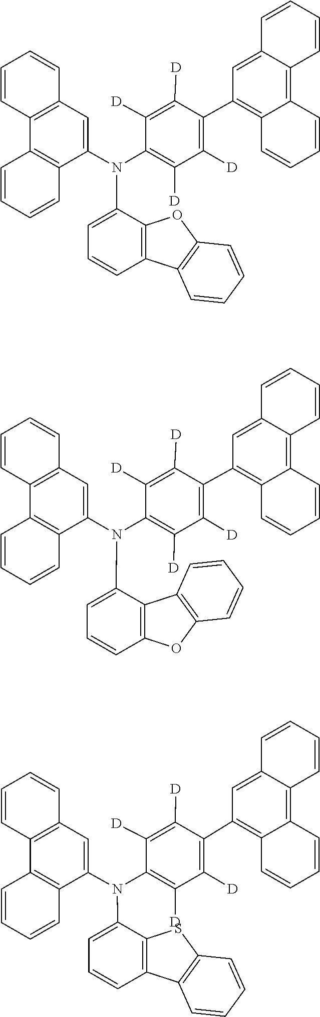 [SMILES: [2H]c1c([2H])c(N(c2cc3ccccc3c3ccccc23)c2cccc3c2oc2ccccc23)c([2H])c([2H])c1-c1cc2ccccc2c2ccccc12 ; [2H]c1c([2H])c(N(c2cc3ccccc3c3ccccc23)c2cccc3oc4ccccc4c23)c([2H])c([2H])c1-c1cc2ccccc2c2ccccc12 ; [2H]c1c([2H])c(N(c2cc3ccccc3c3ccccc23)c2cccc3c2sc2ccccc23)c([2H])c([2H])c1-c1cc2ccccc2c2ccccc12] 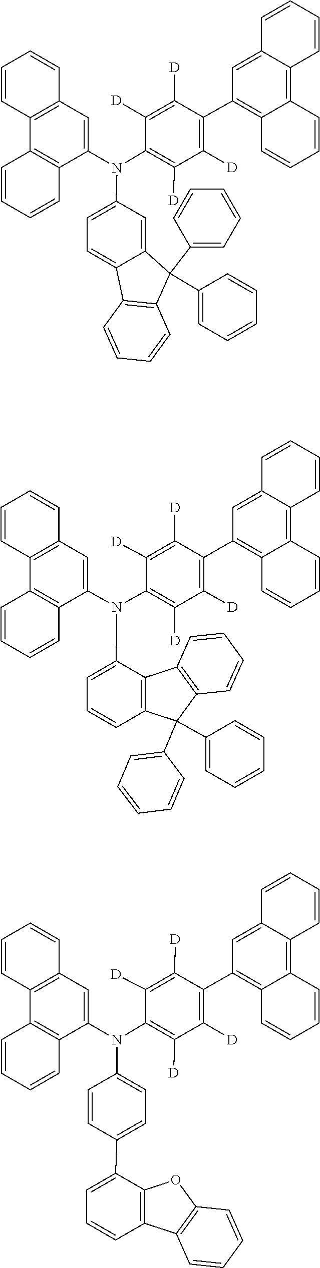 [SMILES: [2H]c1c([2H])c(N(c2ccc3c(c2)C(c2ccccc2)(c2ccccc2)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(c2ccccc2)c2ccccc2)c2cc3ccccc3c3ccccc23)c([2H])c([2H])c1-c1cc2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1cc2ccccc2c2ccccc12] 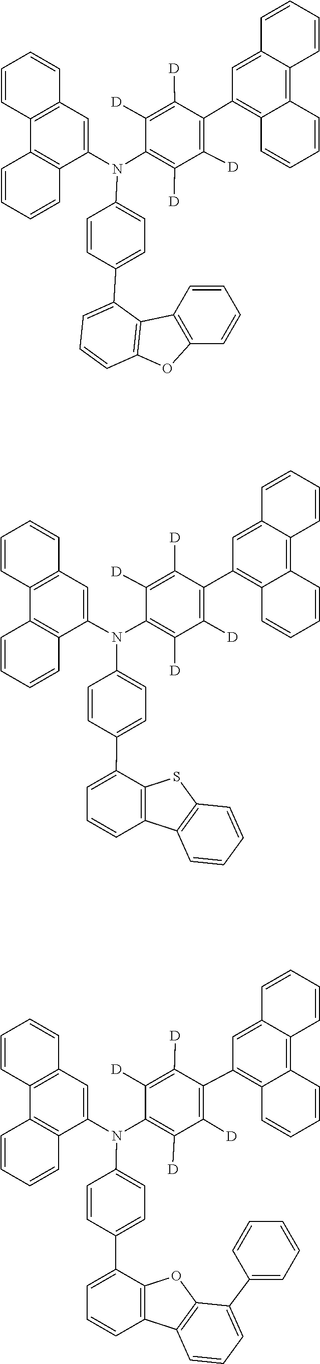 [SMILES: [2H]c1c([2H])c(N(c2ccc(-c3cccc4oc5ccccc5c34)cc2)c2cc3ccccc3c3ccccc23)c([2H])c([2H])c1-c1cc2ccccc2c2ccccc12 ; [2H]c1c([2H])c(N(c2ccc(-c3cccc4c3sc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1cc2ccccc2c2ccccc12] 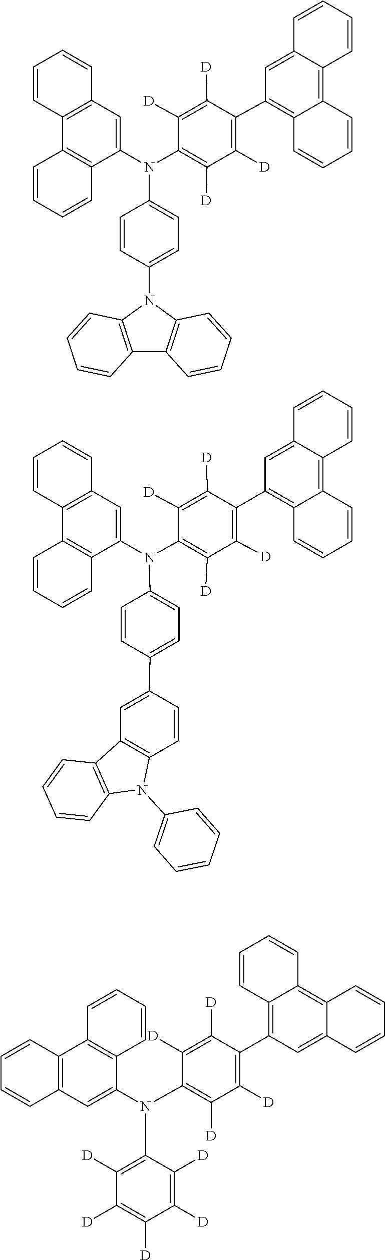 [SMILES: [2H]c1c([2H])c(N(c2ccc(-n3c4ccccc4c4ccccc43)cc2)c2cc3ccccc3c3ccccc23)c([2H])c([2H])c1-c1cc2ccccc2c2ccccc12 ; [2H]c1c([2H])c(N(c2ccc(-c3ccc4c(c3)c3ccccc3n4-c3ccccc3)cc2)c2cc3ccccc3c3ccccc23)c([2H])c([2H])c1-c1cc2ccccc2c2ccccc12 ; [2H]c1c([2H])c([2H])c(N(c2c([2H])c([2H])c(-c3cc4ccccc4c4ccccc34)c([2H])c2[2H])c2cc3ccccc3c3ccccc23)c([2H])c1[2H]] 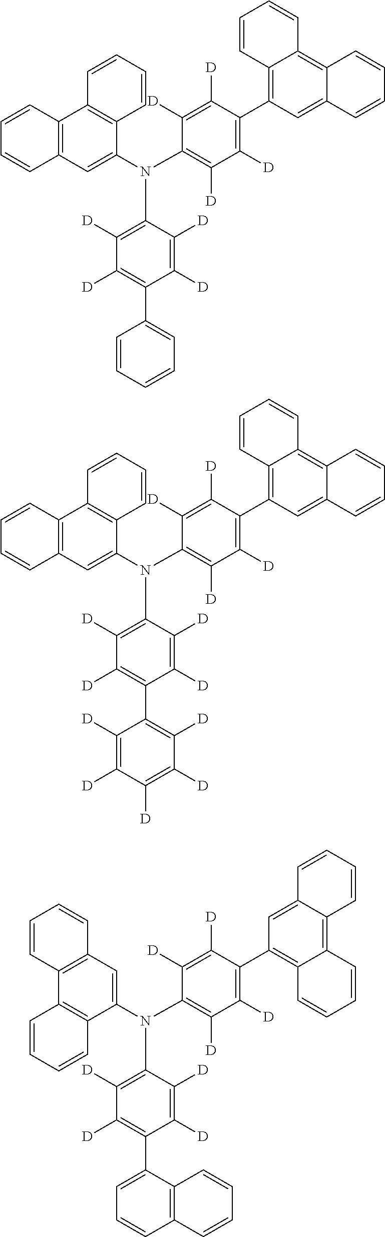 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4cc5ccccc5c5ccccc45)c([2H])c3[2H])c3cc4ccccc4c4ccccc34)c([2H])c2[2H])c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc2ccccc12] 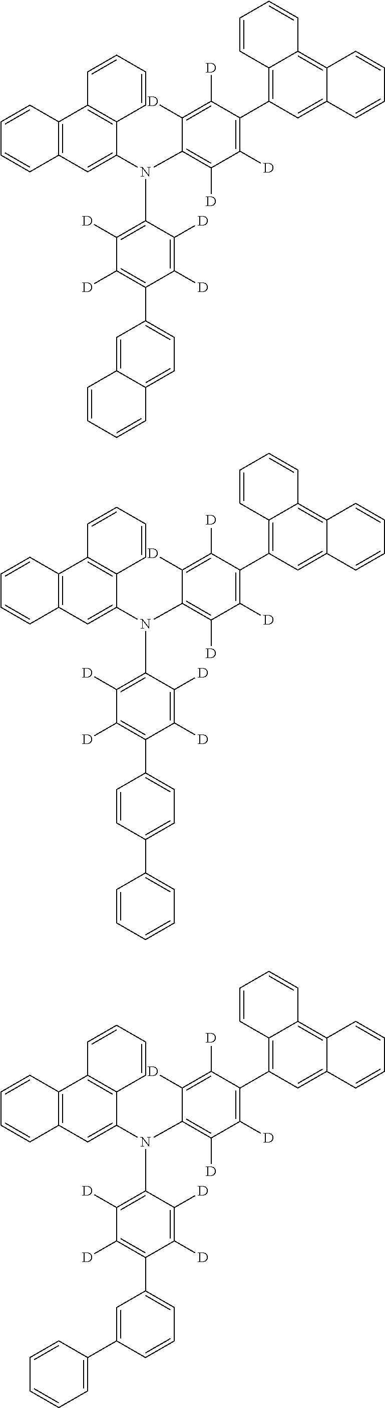 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc(-c2ccccc2)cc1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc(-c2ccccc2)c1] 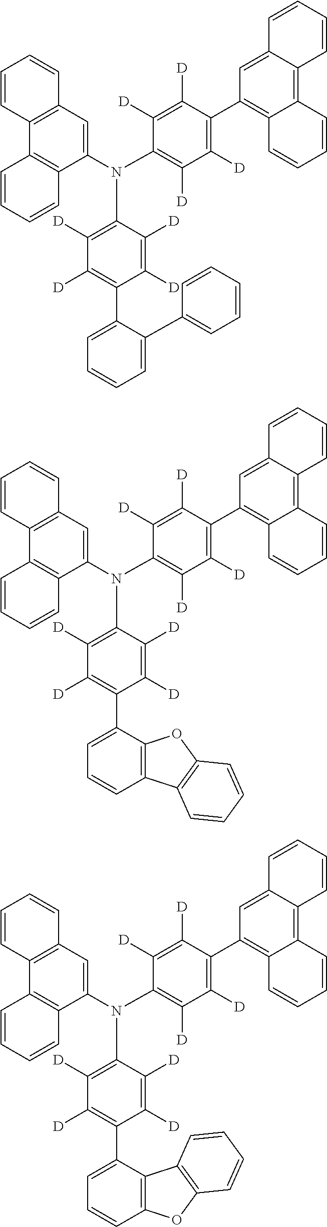 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1-c1ccccc1 ; [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1cc2ccccc2c2ccccc12] 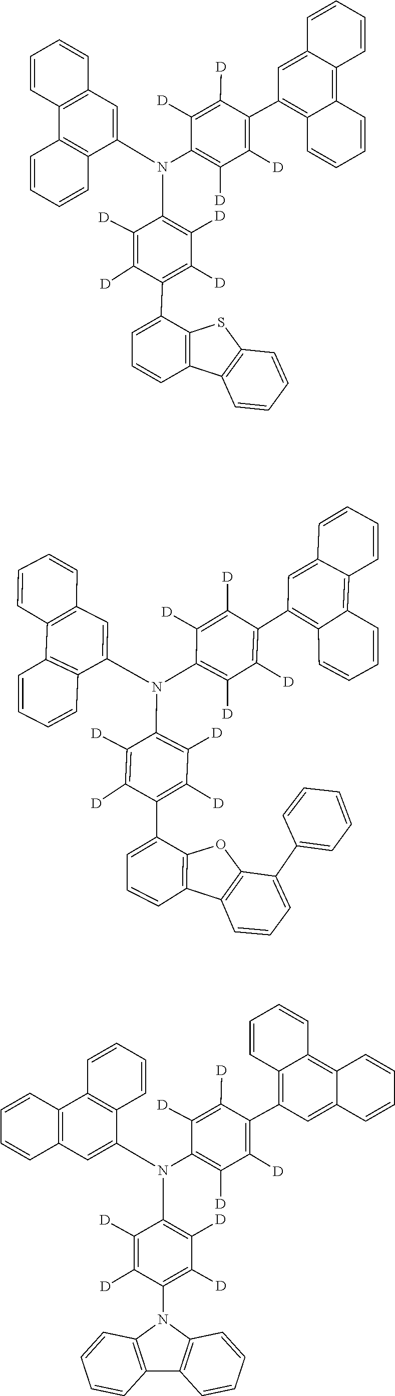 [SMILES: [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1cc2ccccc2c2ccccc12] 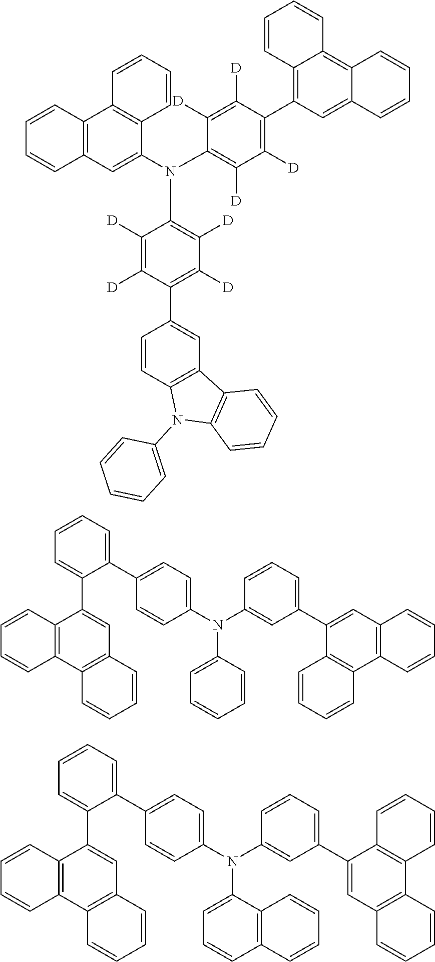 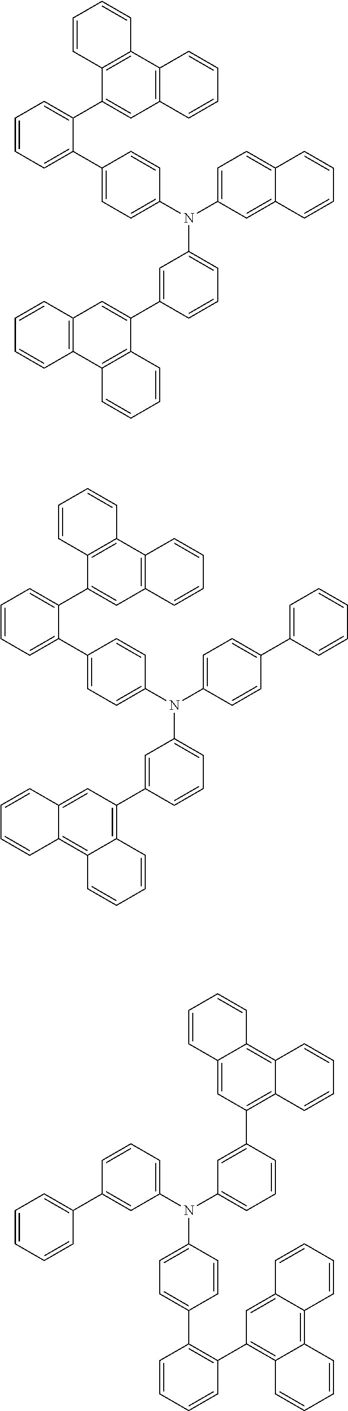 [SMILES: c1cc(-c2cc3ccccc3c3ccccc23)cc(N(c2ccc(-c3ccccc3-c3cc4ccccc4c4ccccc34)cc2)c2ccc3ccccc3c2)c1 ; c1ccc(-c2ccc(N(c3ccc(-c4ccccc4-c4cc5ccccc5c5ccccc45)cc3)c3cccc(-c4cc5ccccc5c5ccccc45)c3)cc2)cc1 ; c1ccc(-c2cccc(N(c3ccc(-c4ccccc4-c4cc5ccccc5c5ccccc45)cc3)c3cccc(-c4cc5ccccc5c5ccccc45)c3)c2)cc1] 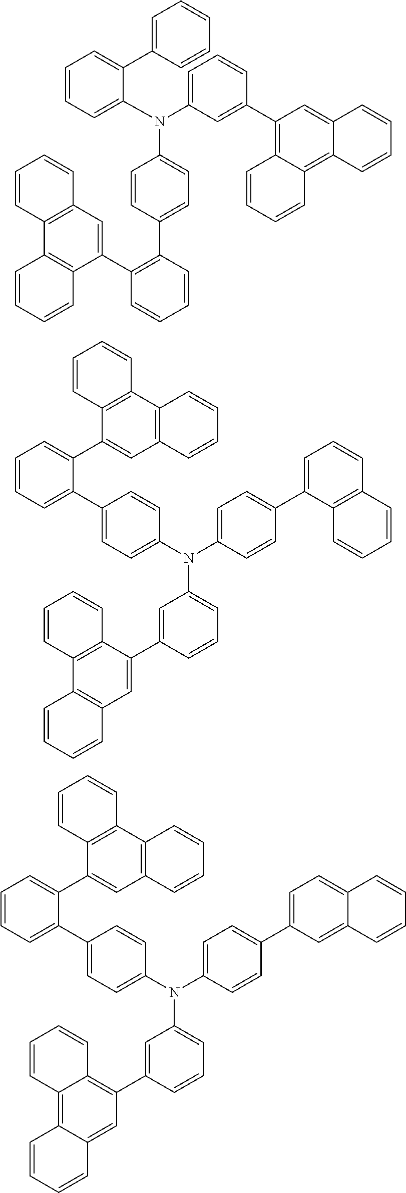 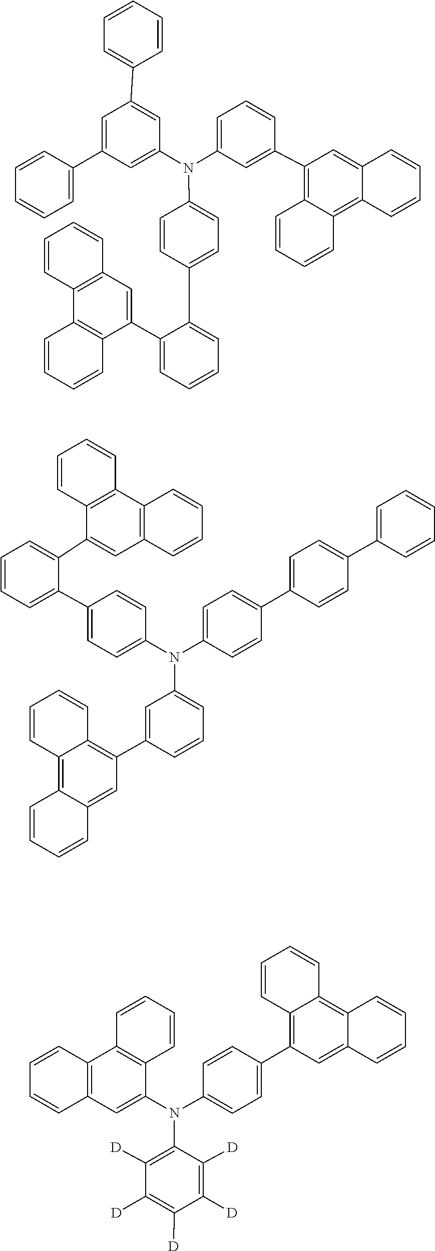 [SMILES: c1ccc(-c2cc(-c3ccccc3)cc(N(c3ccc(-c4ccccc4-c4cc5ccccc5c5ccccc45)cc3)c3cccc(-c4cc5ccccc5c5ccccc45)c3)c2)cc1 ; c1ccc(-c2ccc(-c3ccc(N(c4ccc(-c5ccccc5-c5cc6ccccc6c6ccccc56)cc4)c4cccc(-c5cc6ccccc6c6ccccc56)c4)cc3)cc2)cc1 ; [2H]c1c([2H])c([2H])c(N(c2ccc(-c3cc4ccccc4c4ccccc34)cc2)c2cc3ccccc3c3ccccc23)c([2H])c1[2H]] 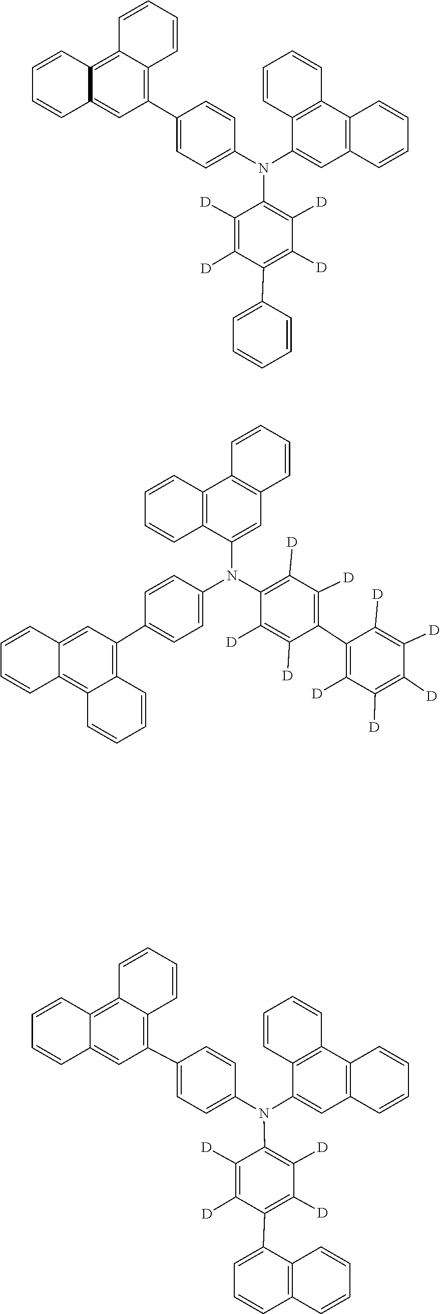 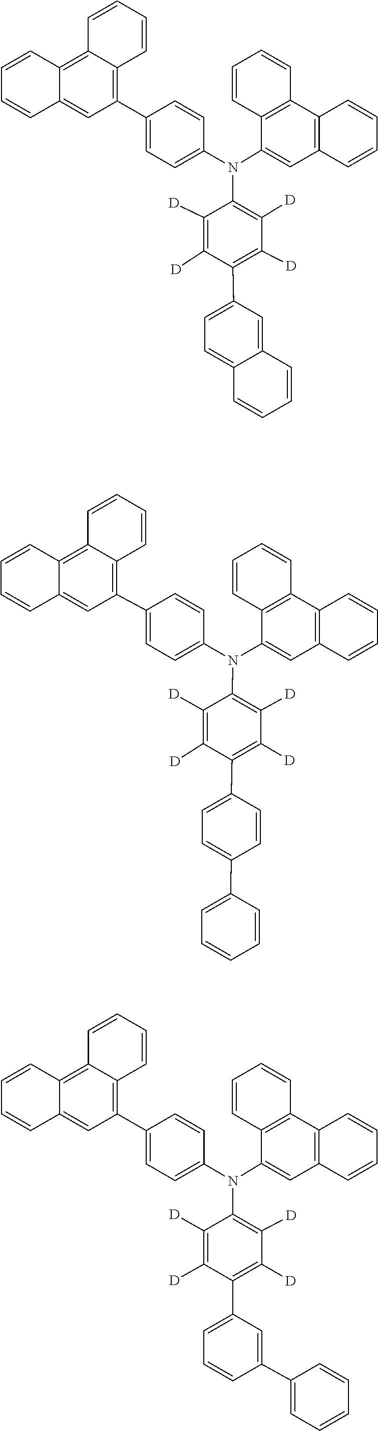 [SMILES: [2H]c1c([2H])c(N(c2ccc(-c3cc4ccccc4c4ccccc34)cc2)c2cc3ccccc3c3ccccc23)c([2H])c([2H])c1-c1ccc2ccccc2c1 ; [2H]c1c([2H])c(N(c2ccc(-c3cc4ccccc4c4ccccc34)cc2)c2cc3ccccc3c3ccccc23)c([2H])c([2H])c1-c1ccc(-c2ccccc2)cc1 ; [2H]c1c([2H])c(N(c2ccc(-c3cc4ccccc4c4ccccc34)cc2)c2cc3ccccc3c3ccccc23)c([2H])c([2H])c1-c1cccc(-c2ccccc2)c1] 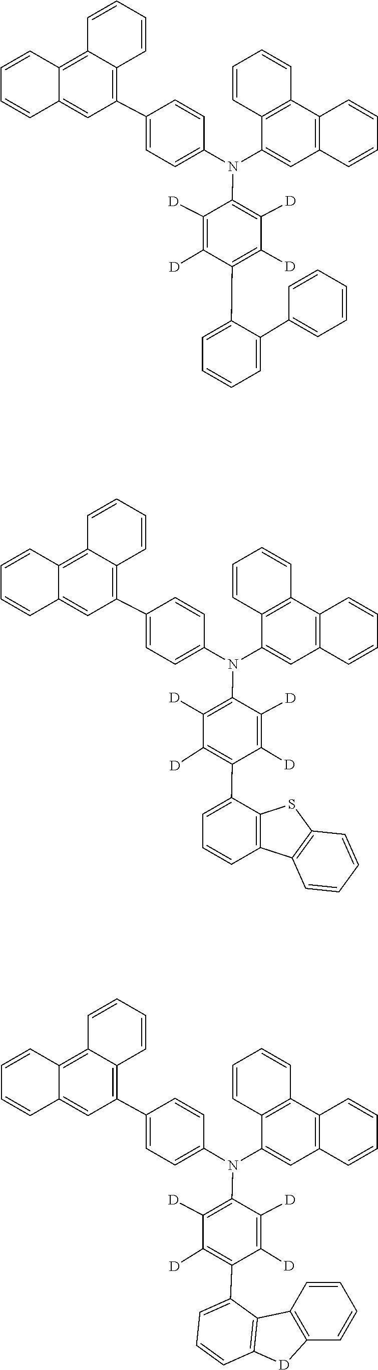 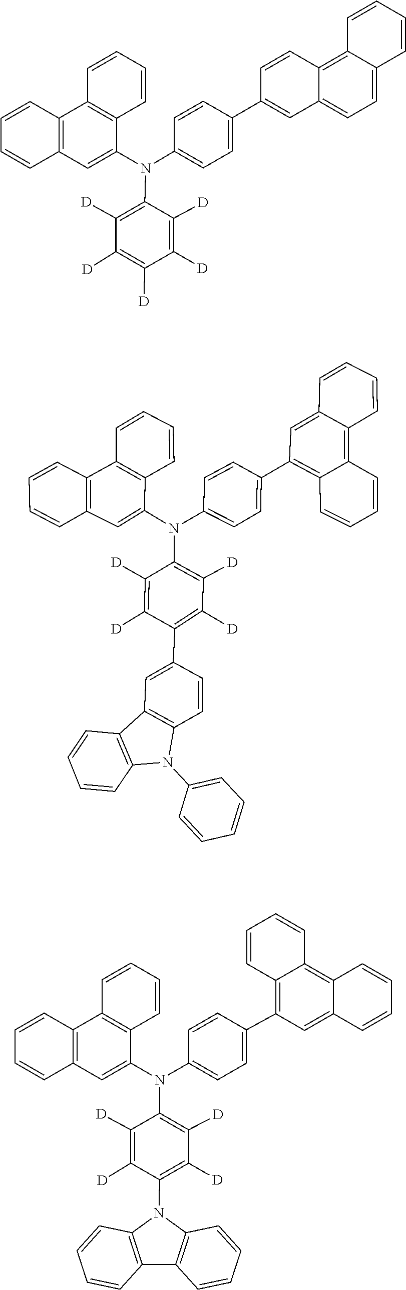 [SMILES: [2H]c1c([2H])c([2H])c(N(c2ccc(-c3ccc4c(ccc5ccccc54)c3)cc2)c2cc3ccccc3c3ccccc23)c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1ccc2c(c1)c1ccccc1n2-c1ccccc1 ; [2H]c1c([2H])c(-n2c3ccccc3c3ccccc32)c([2H])c([2H])c1N(c1ccc(-c2cc3ccccc3c3ccccc23)cc1)c1cc2ccccc2c2ccccc12] 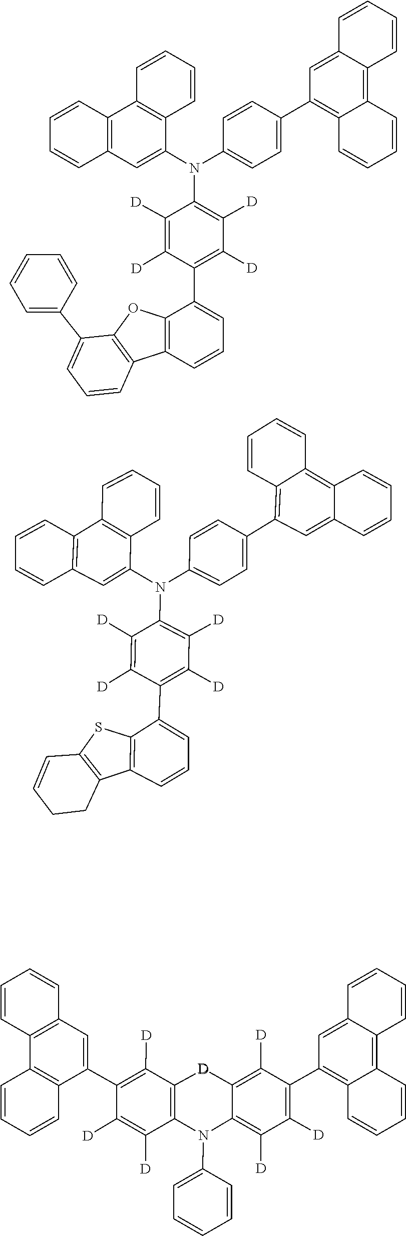 [SMILES: [2H]c1c([2H])c(N(c2ccc(-c3cc4ccccc4c4ccccc34)cc2)c2cc3ccccc3c3ccccc23)c([2H])c([2H])c1-c1cccc2c1oc1c(-c3ccccc3)cccc12 ; [2H]c1c([2H])c(N(c2ccc(-c3cc4ccccc4c4ccccc34)cc2)c2cc3ccccc3c3ccccc23)c([2H])c([2H])c1-c1cccc2c3c(sc12)C=CCC3 ; [2H]c1c([2H])c(N(c2ccccc2)c2c([2H])c([2H])c(-c3cc4ccccc4c4ccccc34)c([2H])c2[2H])c([2H])c([2H])c1-c1cc2ccccc2c2ccccc12] 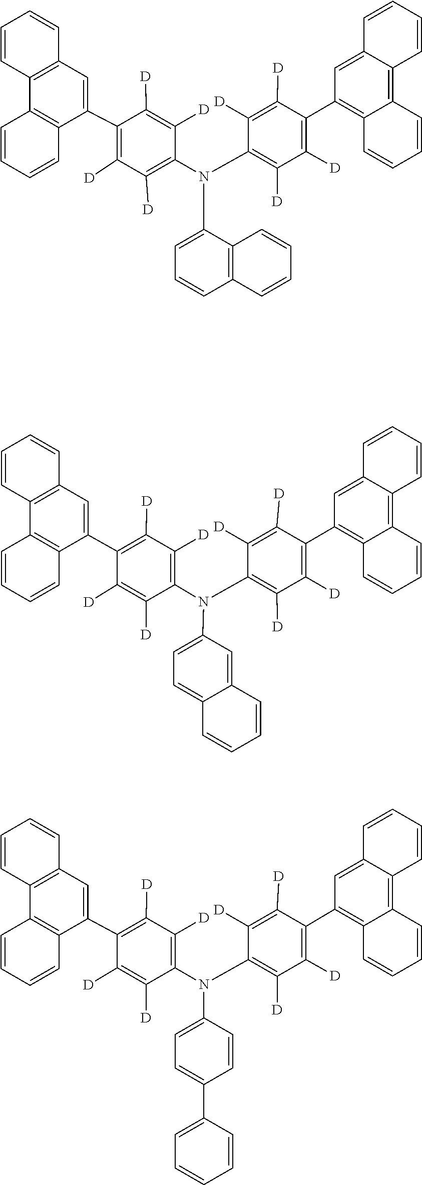 [SMILES: [2H]c1c([2H])c(N(c2c([2H])c([2H])c(-c3cc4ccccc4c4ccccc34)c([2H])c2[2H])c2cccc3ccccc23)c([2H])c([2H])c1-c1cc2ccccc2c2ccccc12 ; [2H]c1c([2H])c(N(c2ccc3ccccc3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccccc3)cc2)c2c([2H])c([2H])c(-c3cc4ccccc4c4ccccc34)c([2H])c2[2H])c([2H])c([2H])c1-c1cc2ccccc2c2ccccc12] 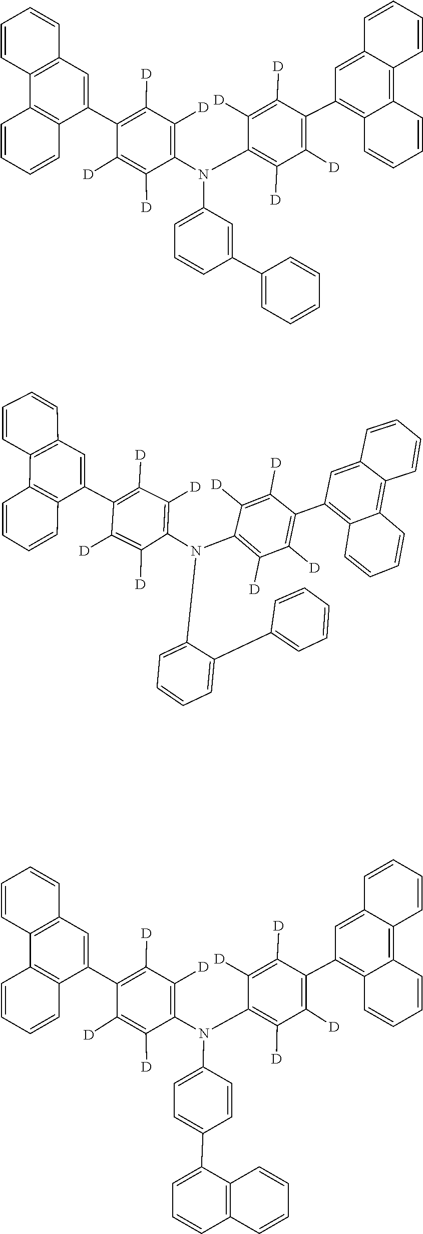 [SMILES: [2H]c1c([2H])c(N(c2cccc(-c3ccccc3)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccccc2-c2ccccc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc4ccccc34)cc2)c2c([2H])c([2H])c(-c3cc4ccccc4c4ccccc34)c([2H])c2[2H])c([2H])c([2H])c1-c1cc2ccccc2c2ccccc12] 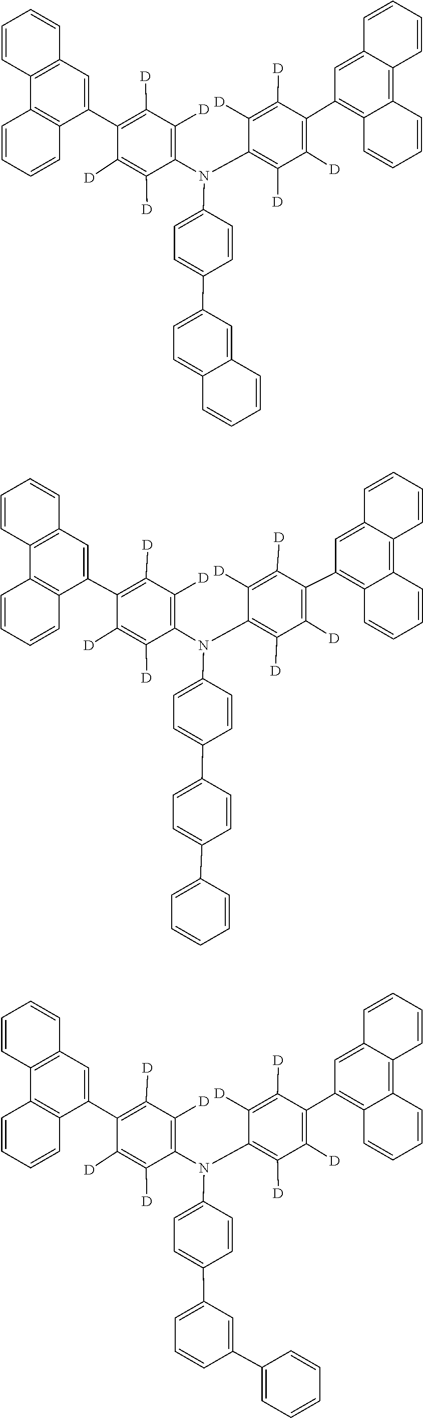 [SMILES: [2H]c1c([2H])c(N(c2ccc(-c3ccc4ccccc4c3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccc(-c4ccccc4)cc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc(-c4ccccc4)c3)cc2)c2c([2H])c([2H])c(-c3cc4ccccc4c4ccccc34)c([2H])c2[2H])c([2H])c([2H])c1-c1cc2ccccc2c2ccccc12] 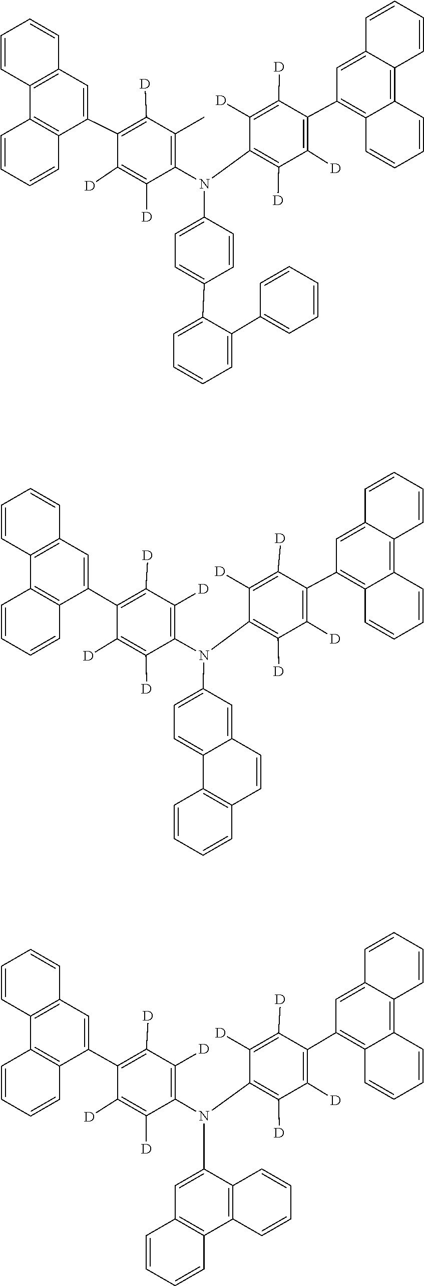 [SMILES: [2H]c1c([2H])c(N(c2ccc(-c3ccccc3-c3ccccc3)cc2)c2c([2H])c([2H])c(-c3cc4ccccc4c4ccccc34)c([2H])c2C)c([2H])c([2H])c1-c1cc2ccccc2c2ccccc12 ; [2H]c1c([2H])c(N(c2ccc3c(ccc4ccccc43)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cc2ccccc2c2ccccc12] 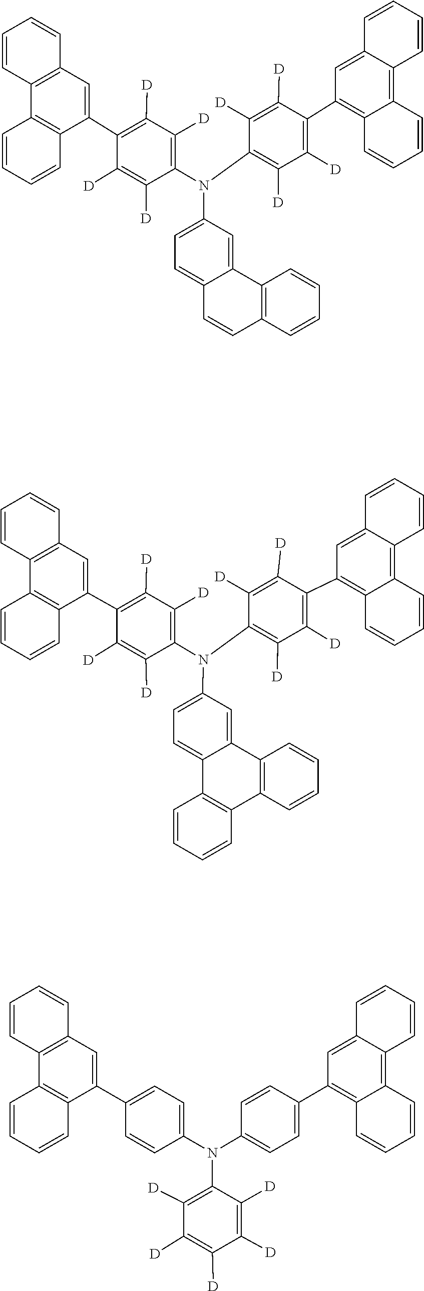 [SMILES: [2H]c1c([2H])c(N(c2ccc3ccc4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c4ccccc4c4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c([2H])c(N(c2ccc(-c3cc4ccccc4c4ccccc34)cc2)c2ccc(-c3cc4ccccc4c4ccccc34)cc2)c([2H])c1[2H]] 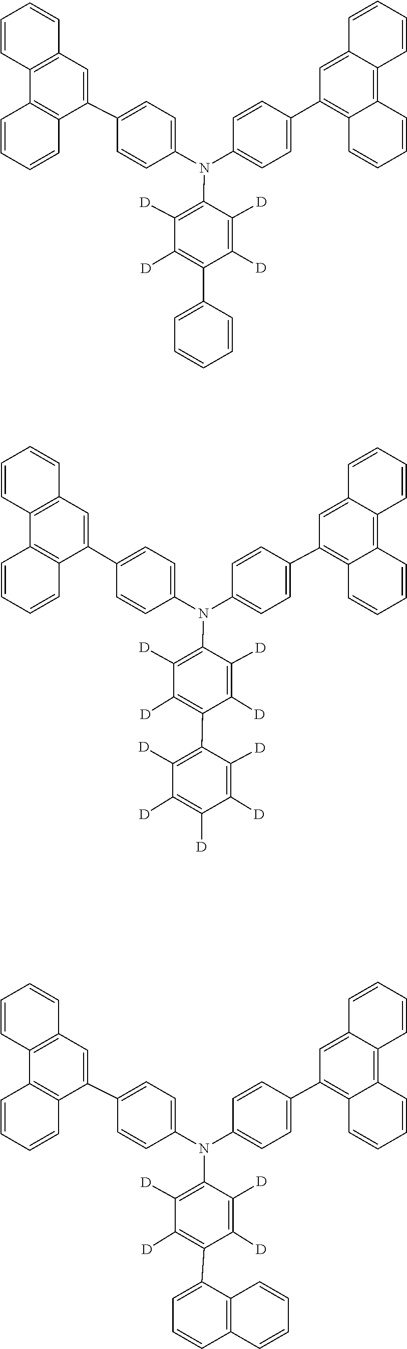 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1 ; [2H]c1c([2H])c([2H])c(-c2c([2H])c([2H])c(N(c3ccc(-c4cc5ccccc5c5ccccc45)cc3)c3ccc(-c4cc5ccccc5c5ccccc45)cc3)c([2H])c2[2H])c([2H])c1[2H] ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1cccc2ccccc12] 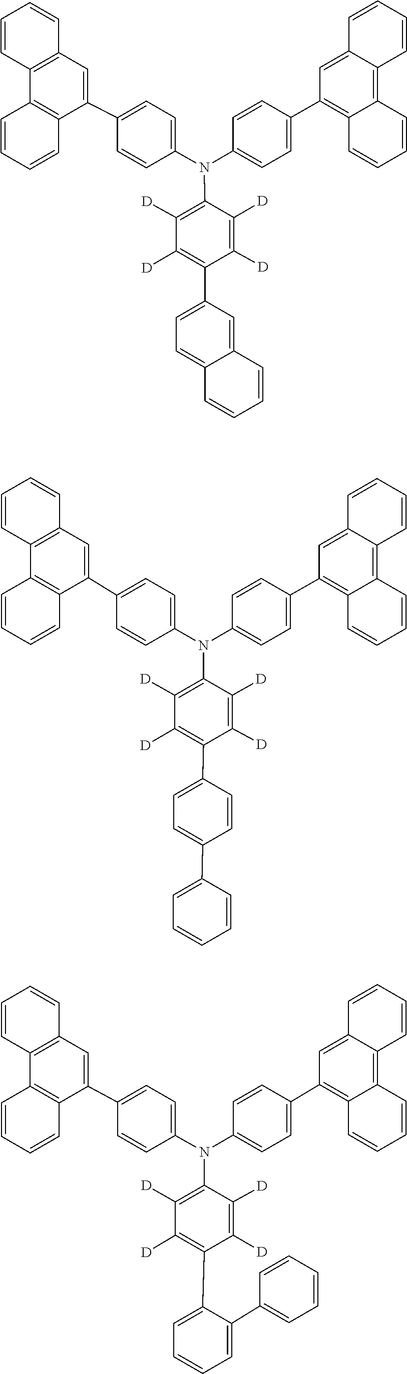 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc2ccccc2c1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc(-c2ccccc2)cc1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1-c1ccccc1] 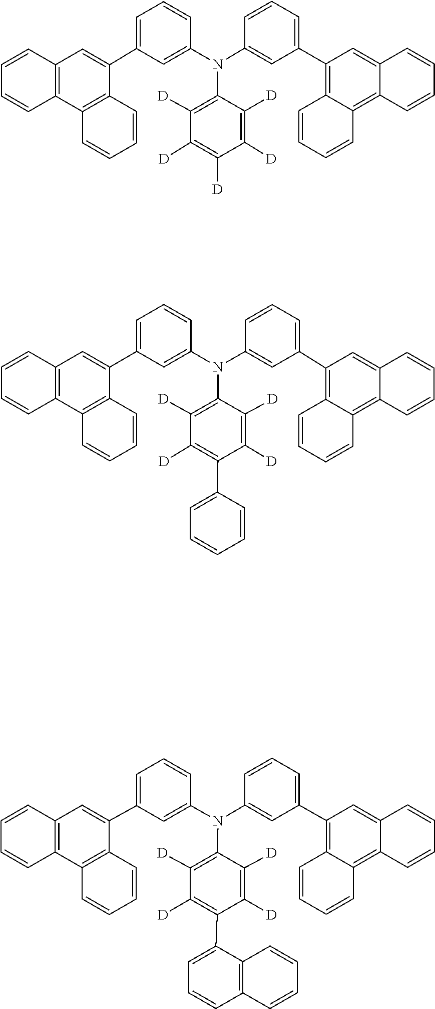 [SMILES: [2H]c1c([2H])c([2H])c(N(c2cccc(-c3cc4ccccc4c4ccccc34)c2)c2cccc(-c3cc4ccccc4c4ccccc34)c2)c([2H])c1[2H] ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccccc1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1cccc2ccccc12] 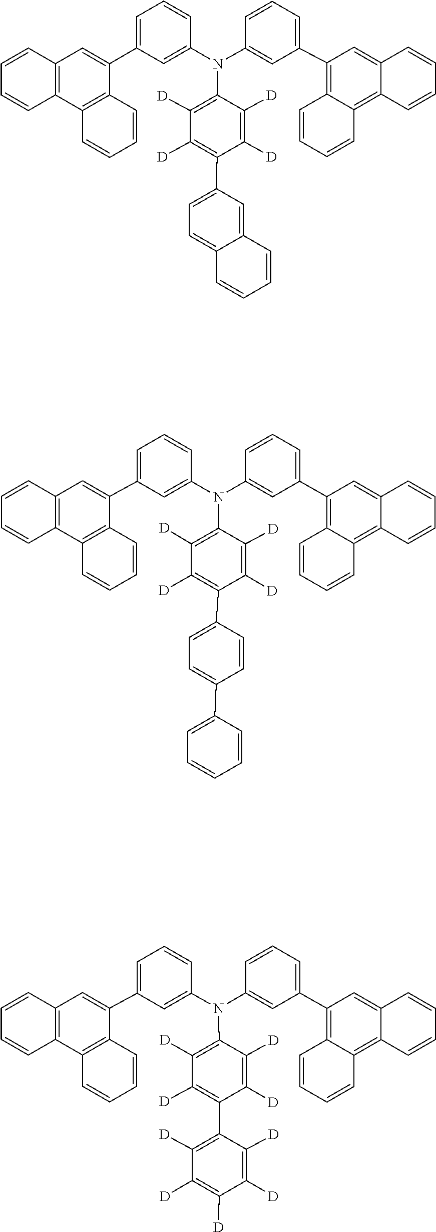 [SMILES: [2H]c1c([2H])c(N(c2cccc(-c3cc4ccccc4c4ccccc34)c2)c2cccc(-c3cc4ccccc4c4ccccc34)c2)c([2H])c([2H])c1-c1ccc2ccccc2c1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccc(-c2ccccc2)cc1 ; [2H]c1c([2H])c([2H])c(-c2c([2H])c([2H])c(N(c3cccc(-c4cc5ccccc5c5ccccc45)c3)c3cccc(-c4cc5ccccc5c5ccccc45)c3)c([2H])c2[2H])c([2H])c1[2H]] 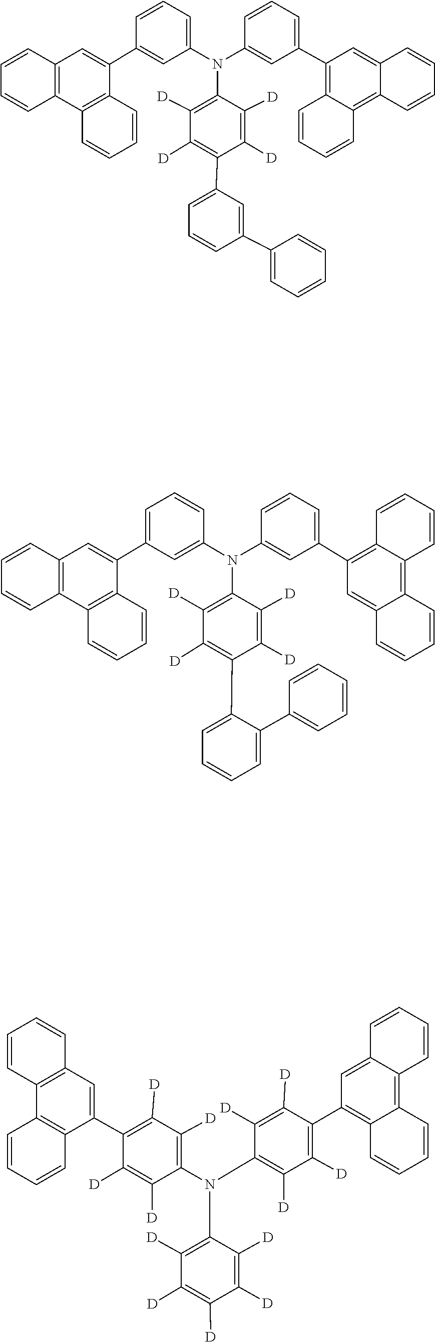 [SMILES: [2H]c1c([2H])c(N(c2cccc(-c3cc4ccccc4c4ccccc34)c2)c2cccc(-c3cc4ccccc4c4ccccc34)c2)c([2H])c([2H])c1-c1cccc(-c2ccccc2)c1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccccc1-c1ccccc1 ; [2H]c1c([2H])c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c1[2H]] 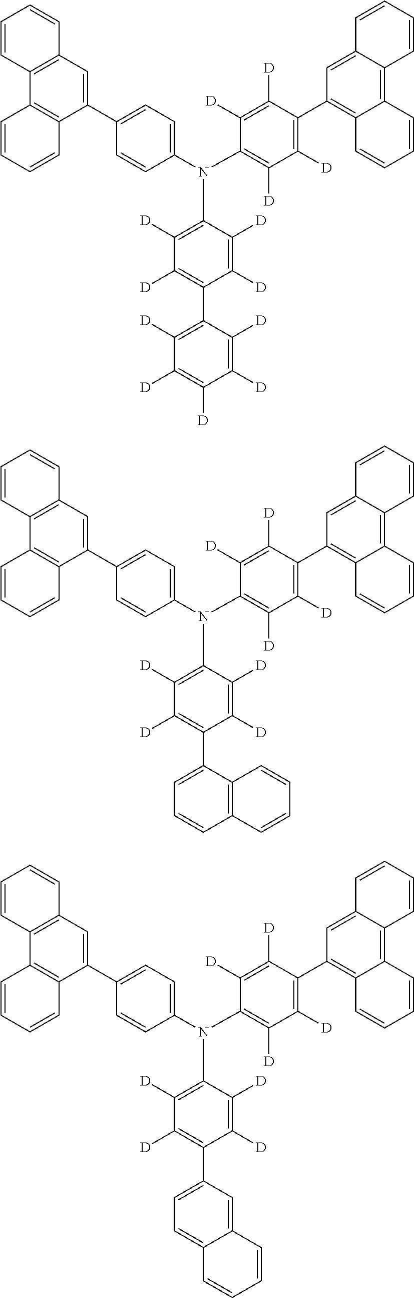 [SMILES: [2H]c1c([2H])c([2H])c(-c2c([2H])c([2H])c(N(c3ccc(-c4cc5ccccc5c5ccccc45)cc3)c3c([2H])c([2H])c(-c4cc5ccccc5c5ccccc45)c([2H])c3[2H])c([2H])c2[2H])c([2H])c1[2H] ; [2H]c1c([2H])c(N(c2ccc(-c3cc4ccccc4c4ccccc34)cc2)c2c([2H])c([2H])c(-c3cc4ccccc4c4ccccc34)c([2H])c2[2H])c([2H])c([2H])c1-c1cccc2ccccc12 ; [2H]c1c([2H])c(N(c2ccc(-c3cc4ccccc4c4ccccc34)cc2)c2c([2H])c([2H])c(-c3cc4ccccc4c4ccccc34)c([2H])c2[2H])c([2H])c([2H])c1-c1ccc2ccccc2c1] 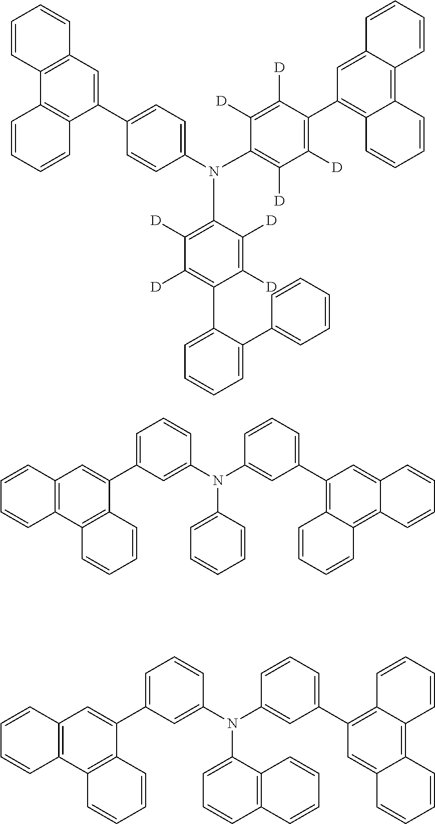 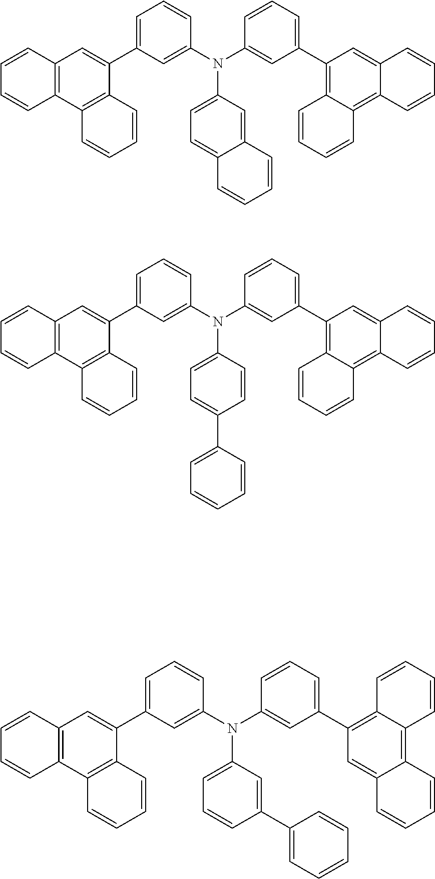 [SMILES: c1cc(-c2cc3ccccc3c3ccccc23)cc(N(c2cccc(-c3cc4ccccc4c4ccccc34)c2)c2ccc3ccccc3c2)c1 ; c1ccc(-c2ccc(N(c3cccc(-c4cc5ccccc5c5ccccc45)c3)c3cccc(-c4cc5ccccc5c5ccccc45)c3)cc2)cc1 ; c1ccc(-c2cccc(N(c3cccc(-c4cc5ccccc5c5ccccc45)c3)c3cccc(-c4cc5ccccc5c5ccccc45)c3)c2)cc1] 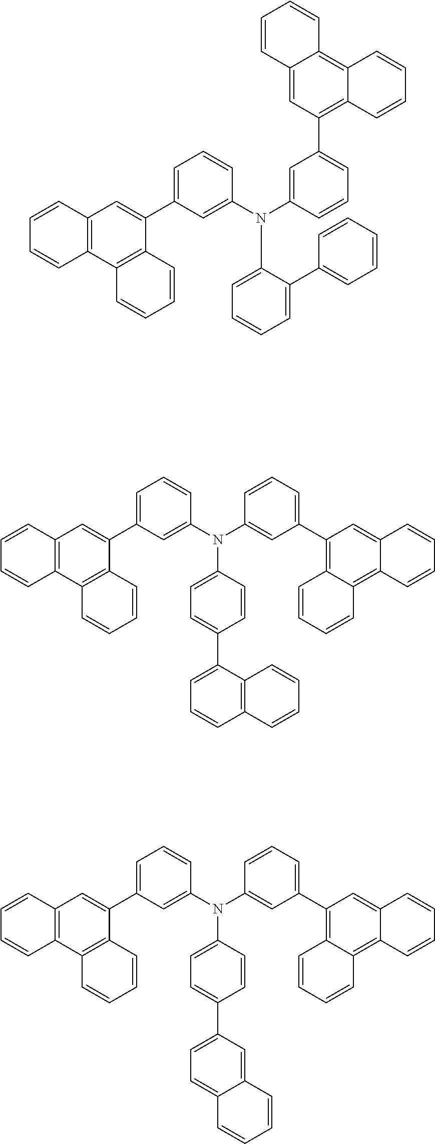 [SMILES: c1ccc(-c2ccccc2N(c2cccc(-c3cc4ccccc4c4ccccc34)c2)c2cccc(-c3cc4ccccc4c4ccccc34)c2)cc1 ; c1cc(-c2cc3ccccc3c3ccccc23)cc(N(c2ccc(-c3cccc4ccccc34)cc2)c2cccc(-c3cc4ccccc4c4ccccc34)c2)c1 ; c1cc(-c2cc3ccccc3c3ccccc23)cc(N(c2ccc(-c3ccc4ccccc4c3)cc2)c2cccc(-c3cc4ccccc4c4ccccc34)c2)c1] 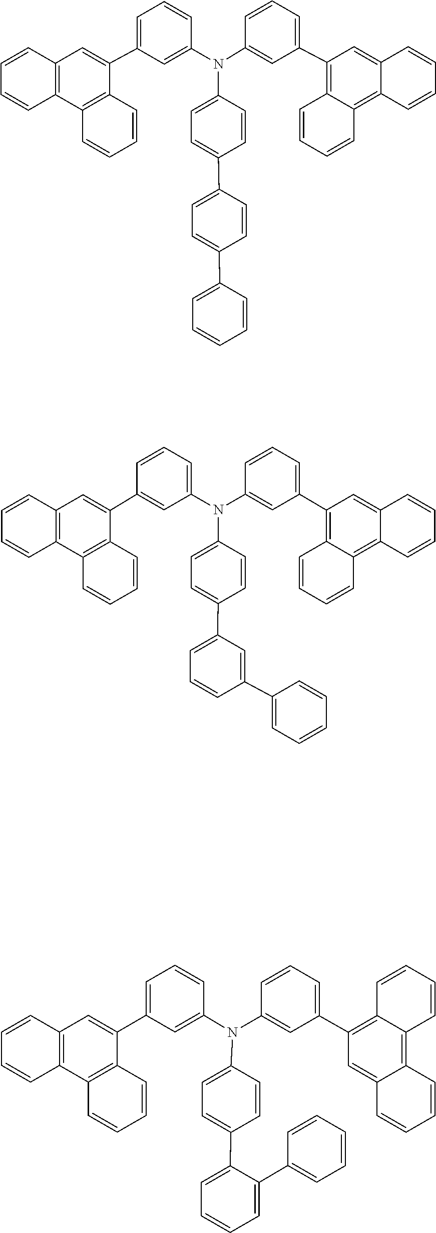 [SMILES: c1ccc(-c2ccc(-c3ccc(N(c4cccc(-c5cc6ccccc6c6ccccc56)c4)c4cccc(-c5cc6ccccc6c6ccccc56)c4)cc3)cc2)cc1 ; c1ccc(-c2cccc(-c3ccc(N(c4cccc(-c5cc6ccccc6c6ccccc56)c4)c4cccc(-c5cc6ccccc6c6ccccc56)c4)cc3)c2)cc1 ; c1ccc(-c2ccccc2-c2ccc(N(c3cccc(-c4cc5ccccc5c5ccccc45)c3)c3cccc(-c4cc5ccccc5c5ccccc45)c3)cc2)cc1] 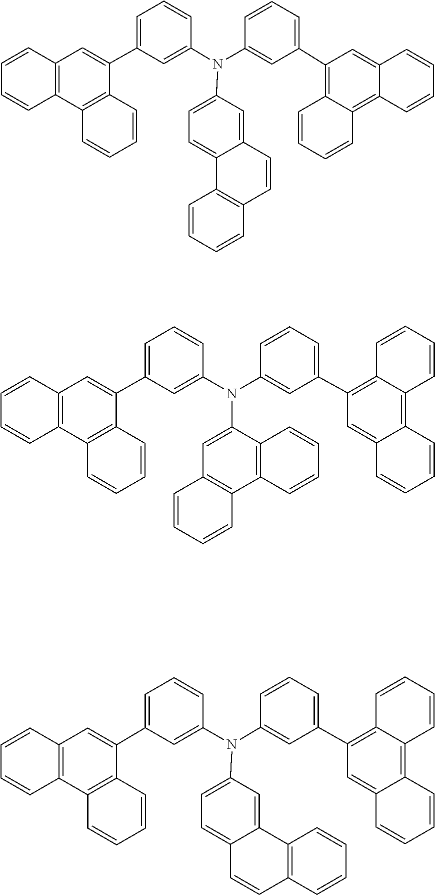 [SMILES: c1cc(-c2cc3ccccc3c3ccccc23)cc(N(c2cccc(-c3cc4ccccc4c4ccccc34)c2)c2ccc3c(ccc4ccccc43)c2)c1 ; c1cc(-c2cc3ccccc3c3ccccc23)cc(N(c2cccc(-c3cc4ccccc4c4ccccc34)c2)c2cc3ccccc3c3ccccc23)c1 ; c1cc(-c2cc3ccccc3c3ccccc23)cc(N(c2cccc(-c3cc4ccccc4c4ccccc34)c2)c2ccc3ccc4ccccc4c3c2)c1] 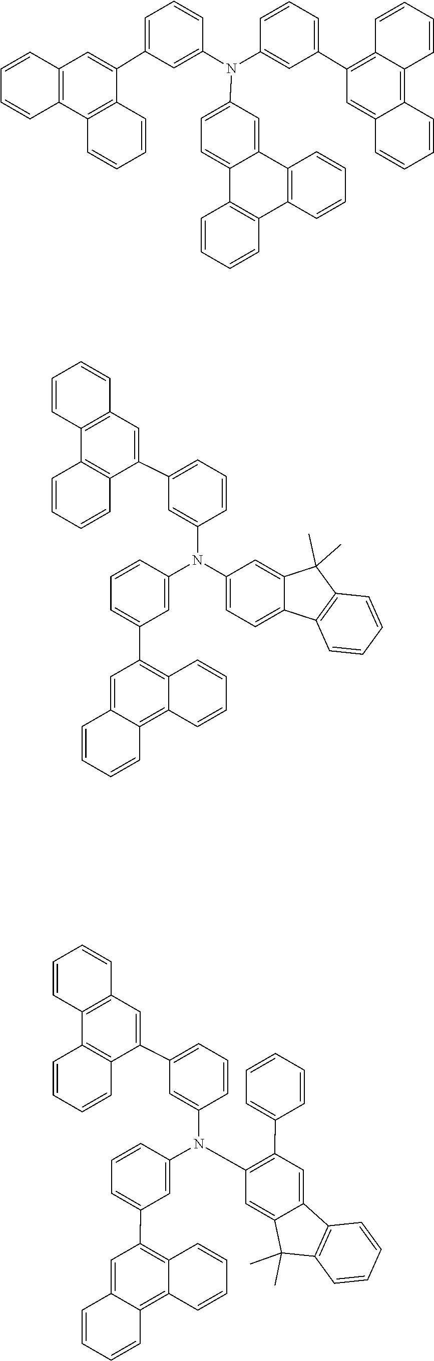 [SMILES: c1cc(-c2cc3ccccc3c3ccccc23)cc(N(c2cccc(-c3cc4ccccc4c4ccccc34)c2)c2ccc3c4ccccc4c4ccccc4c3c2)c1 ; CC1(C)c2ccccc2-c2ccc(N(c3cccc(-c4cc5ccccc5c5ccccc45)c3)c3cccc(-c4cc5ccccc5c5ccccc45)c3)cc21 ; CC1(C)c2ccccc2-c2cc(-c3ccccc3)c(N(c3cccc(-c4cc5ccccc5c5ccccc45)c3)c3cccc(-c4cc5ccccc5c5ccccc45)c3)cc21] 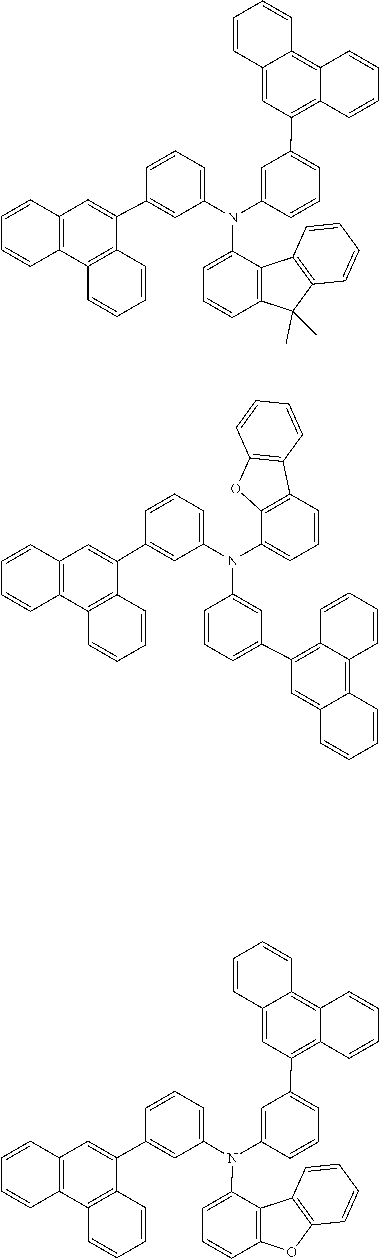 [SMILES: CC1(C)c2ccccc2-c2c(N(c3cccc(-c4cc5ccccc5c5ccccc45)c3)c3cccc(-c4cc5ccccc5c5ccccc45)c3)cccc21 ; c1cc(-c2cc3ccccc3c3ccccc23)cc(N(c2cccc(-c3cc4ccccc4c4ccccc34)c2)c2cccc3c2oc2ccccc23)c1 ; c1cc(-c2cc3ccccc3c3ccccc23)cc(N(c2cccc(-c3cc4ccccc4c4ccccc34)c2)c2cccc3oc4ccccc4c23)c1] 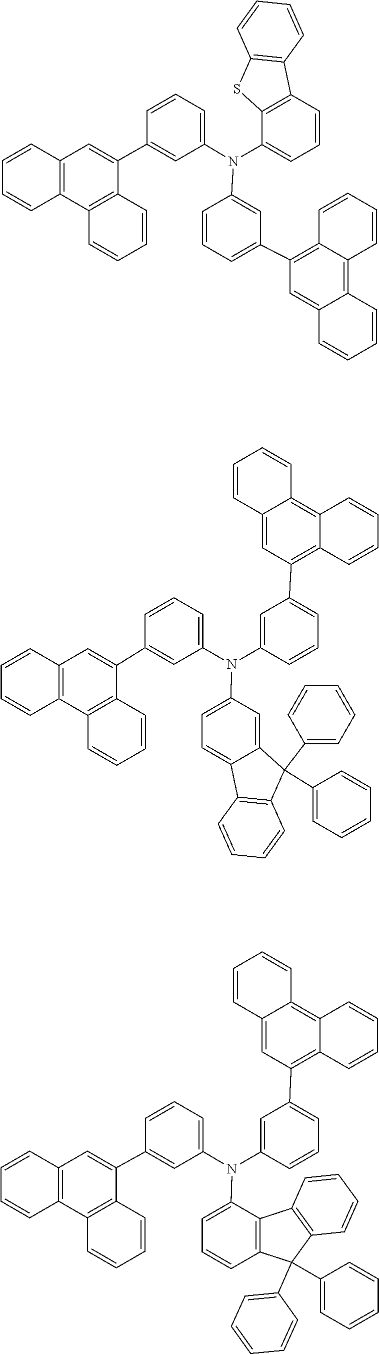 [SMILES: c1cc(-c2cc3ccccc3c3ccccc23)cc(N(c2cccc(-c3cc4ccccc4c4ccccc34)c2)c2cccc3c2sc2ccccc23)c1 ; c1ccc(C2(c3ccccc3)c3ccccc3-c3ccc(N(c4cccc(-c5cc6ccccc6c6ccccc56)c4)c4cccc(-c5cc6ccccc6c6ccccc56)c4)cc32)cc1 ; c1ccc(C2(c3ccccc3)c3ccccc3-c3c(N(c4cccc(-c5cc6ccccc6c6ccccc56)c4)c4cccc(-c5cc6ccccc6c6ccccc56)c4)cccc32)cc1] 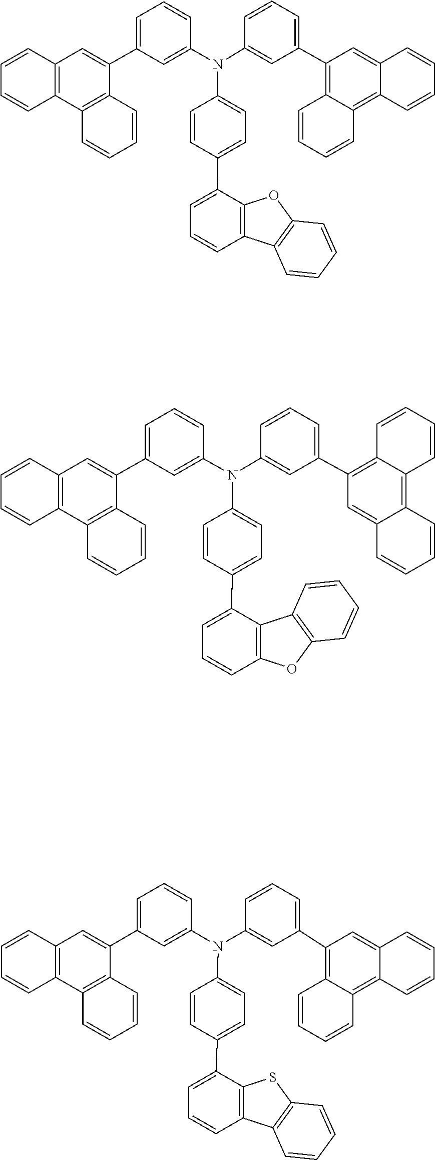 [SMILES: c1cc(-c2cc3ccccc3c3ccccc23)cc(N(c2ccc(-c3cccc4c3oc3ccccc34)cc2)c2cccc(-c3cc4ccccc4c4ccccc34)c2)c1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1 ; c1cc(-c2cc3ccccc3c3ccccc23)cc(N(c2ccc(-c3cccc4c3sc3ccccc34)cc2)c2cccc(-c3cc4ccccc4c4ccccc34)c2)c1] 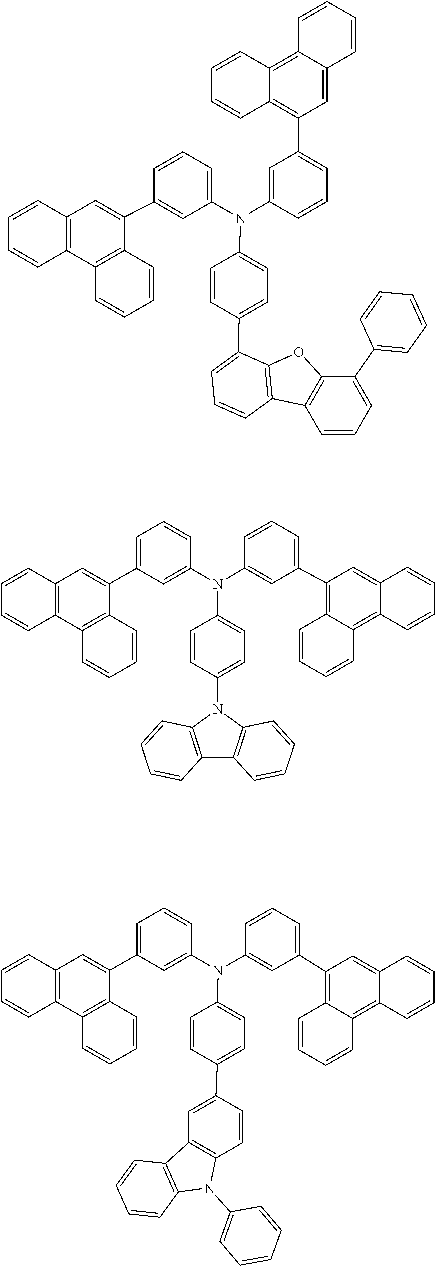 [SMILES: c1ccc(-c2cccc3c2oc2c(-c4ccc(N(c5cccc(-c6cc7ccccc7c7ccccc67)c5)c5cccc(-c6cc7ccccc7c7ccccc67)c5)cc4)cccc23)cc1 ; c1cc(-c2cc3ccccc3c3ccccc23)cc(N(c2ccc(-n3c4ccccc4c4ccccc43)cc2)c2cccc(-c3cc4ccccc4c4ccccc34)c2)c1 ; c1ccc(-n2c3ccccc3c3cc(-c4ccc(N(c5cccc(-c6cc7ccccc7c7ccccc67)c5)c5cccc(-c6cc7ccccc7c7ccccc67)c5)cc4)ccc32)cc1] 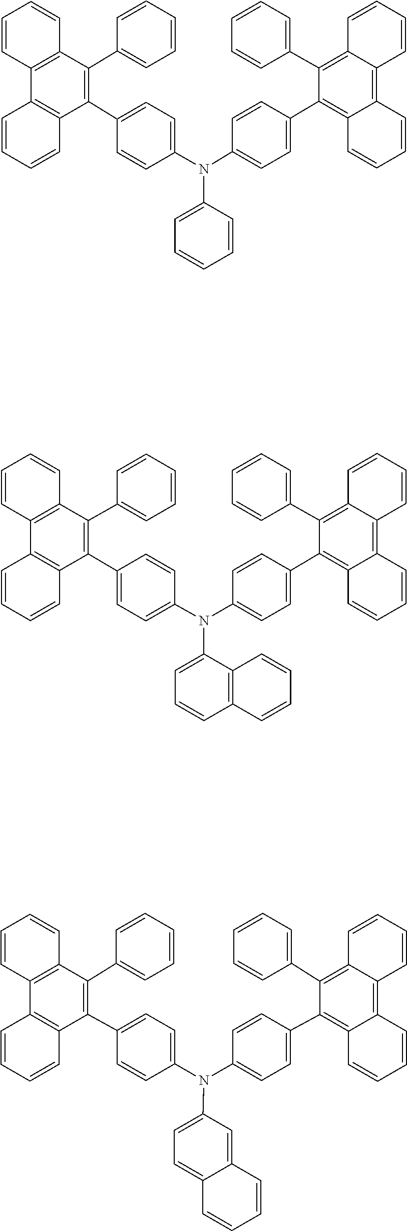 [SMILES: c1ccc(-c2c(-c3ccc(N(c4ccccc4)c4ccc(-c5c(-c6ccccc6)c6ccccc6c6ccccc56)cc4)cc3)c3ccccc3c3ccccc23)cc1 ; c1ccc(-c2c(-c3ccc(N(c4ccc(-c5c(-c6ccccc6)c6ccccc6c6ccccc56)cc4)c4cccc5ccccc45)cc3)c3ccccc3c3ccccc23)cc1 ; c1ccc(-c2c(-c3ccc(N(c4ccc(-c5c(-c6ccccc6)c6ccccc6c6ccccc56)cc4)c4ccc5ccccc5c4)cc3)c3ccccc3c3ccccc23)cc1] 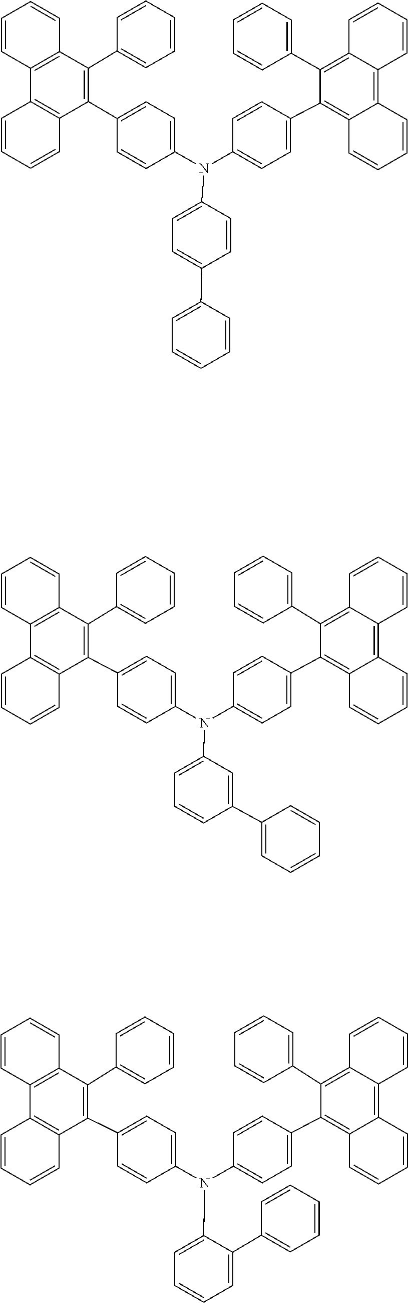 [SMILES: c1ccc(-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1 ; c1ccc(-c2cccc(N(c3ccc(-c4c(-c5ccccc5)c5ccccc5c5ccccc45)cc3)c3ccc(-c4c(-c5ccccc5)c5ccccc5c5ccccc45)cc3)c2)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1] 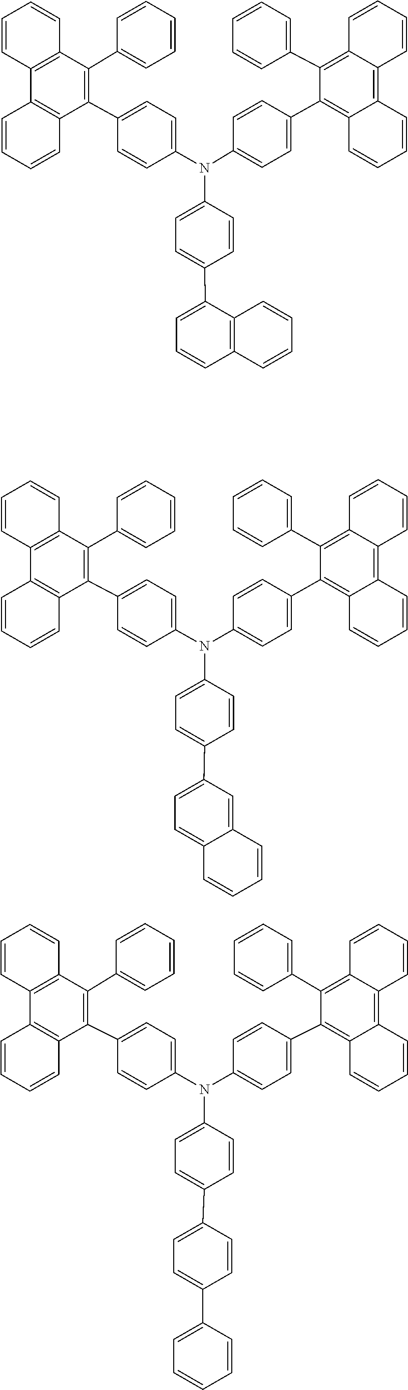 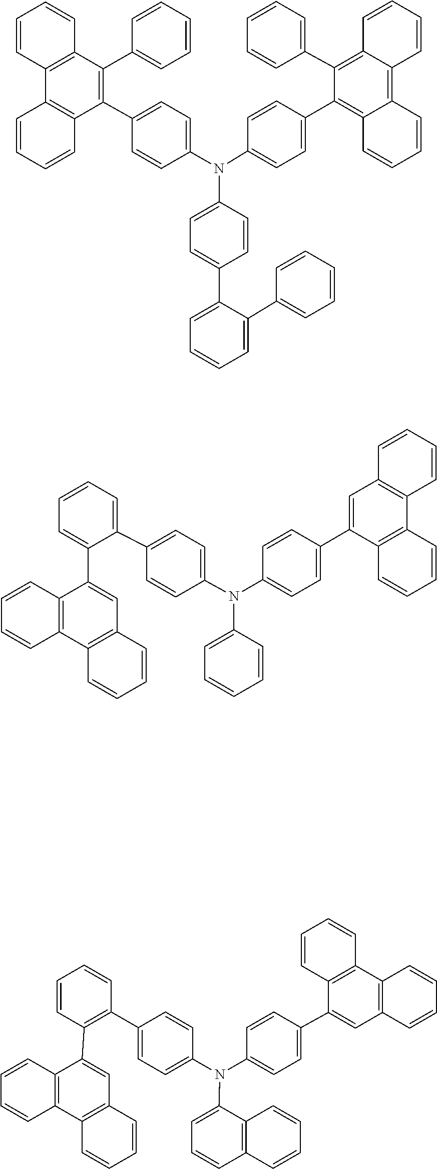 [SMILES: c1ccc(-c2ccccc2-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1 ; c1ccc(N(c2ccc(-c3ccccc3-c3cc4ccccc4c4ccccc34)cc2)c2ccc(-c3cc4ccccc4c4ccccc34)cc2)cc1 ; c1ccc(-c2cc3ccccc3c3ccccc23)c(-c2ccc(N(c3ccc(-c4cc5ccccc5c5ccccc45)cc3)c3cccc4ccccc34)cc2)c1] 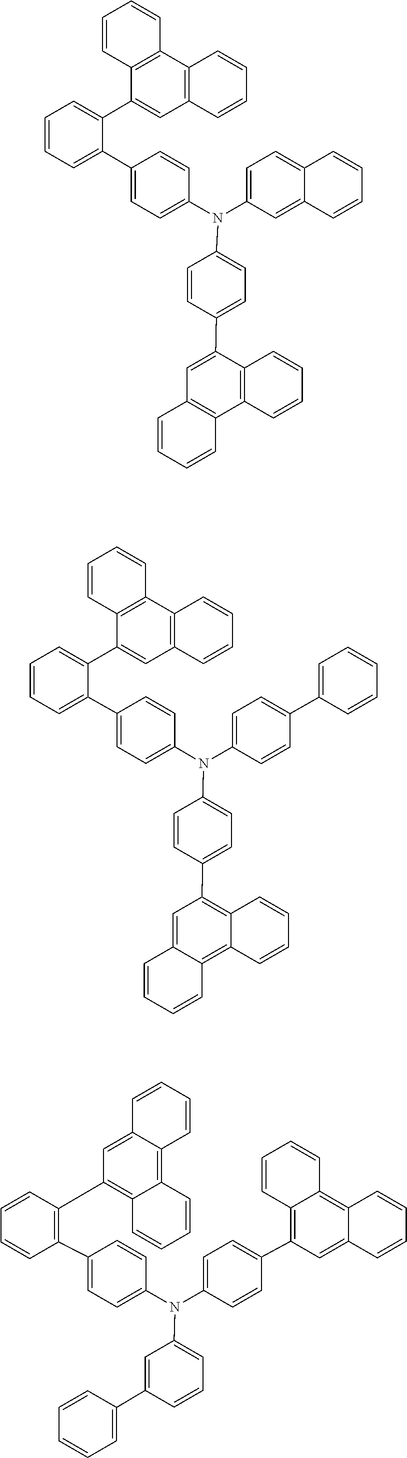 [SMILES: c1ccc(-c2cc3ccccc3c3ccccc23)c(-c2ccc(N(c3ccc(-c4cc5ccccc5c5ccccc45)cc3)c3ccc4ccccc4c3)cc2)c1 ; c1ccc(-c2ccc(N(c3ccc(-c4ccccc4-c4cc5ccccc5c5ccccc45)cc3)c3ccc(-c4cc5ccccc5c5ccccc45)cc3)cc2)cc1 ; c1ccc(-c2cccc(N(c3ccc(-c4ccccc4-c4cc5ccccc5c5ccccc45)cc3)c3ccc(-c4cc5ccccc5c5ccccc45)cc3)c2)cc1] 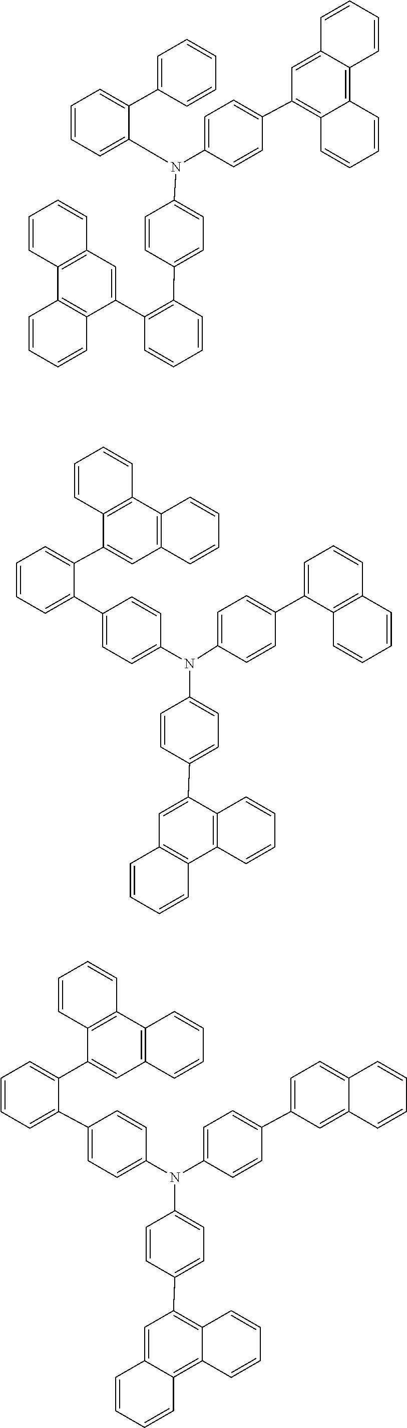 [SMILES: c1ccc(-c2ccccc2N(c2ccc(-c3ccccc3-c3cc4ccccc4c4ccccc34)cc2)c2ccc(-c3cc4ccccc4c4ccccc34)cc2)cc1 ; c1ccc(-c2cc3ccccc3c3ccccc23)c(-c2ccc(N(c3ccc(-c4cccc5ccccc45)cc3)c3ccc(-c4cc5ccccc5c5ccccc45)cc3)cc2)c1 ; c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1] 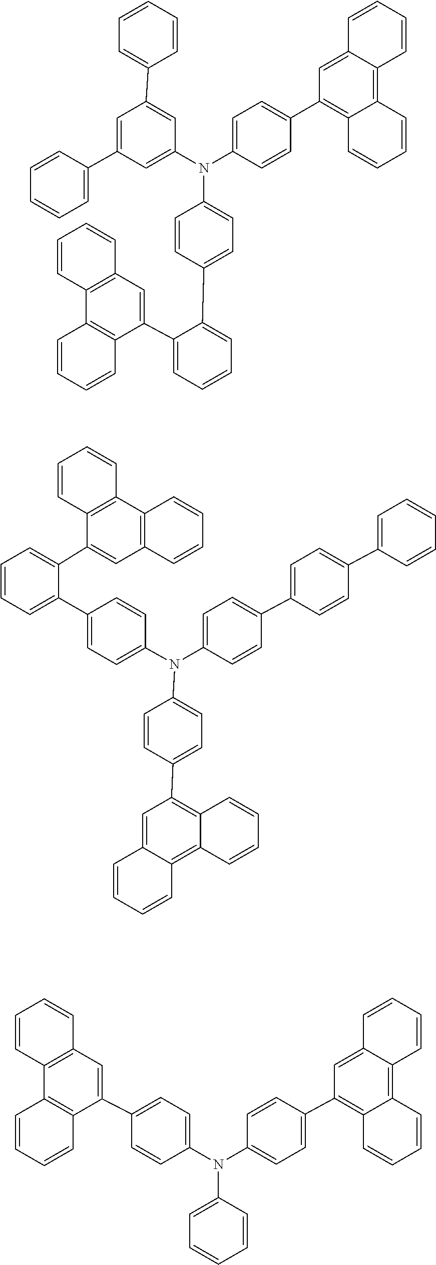 [SMILES: c1ccc(-c2cc(-c3ccccc3)cc(N(c3ccc(-c4ccccc4-c4cc5ccccc5c5ccccc45)cc3)c3ccc(-c4cc5ccccc5c5ccccc45)cc3)c2)cc1 ; c1ccc(-c2ccc(-c3ccc(N(c4ccc(-c5ccccc5-c5cc6ccccc6c6ccccc56)cc4)c4ccc(-c5cc6ccccc6c6ccccc56)cc4)cc3)cc2)cc1 ; c1ccc(N(c2ccc(-c3cc4ccccc4c4ccccc34)cc2)c2ccc(-c3cc4ccccc4c4ccccc34)cc2)cc1] 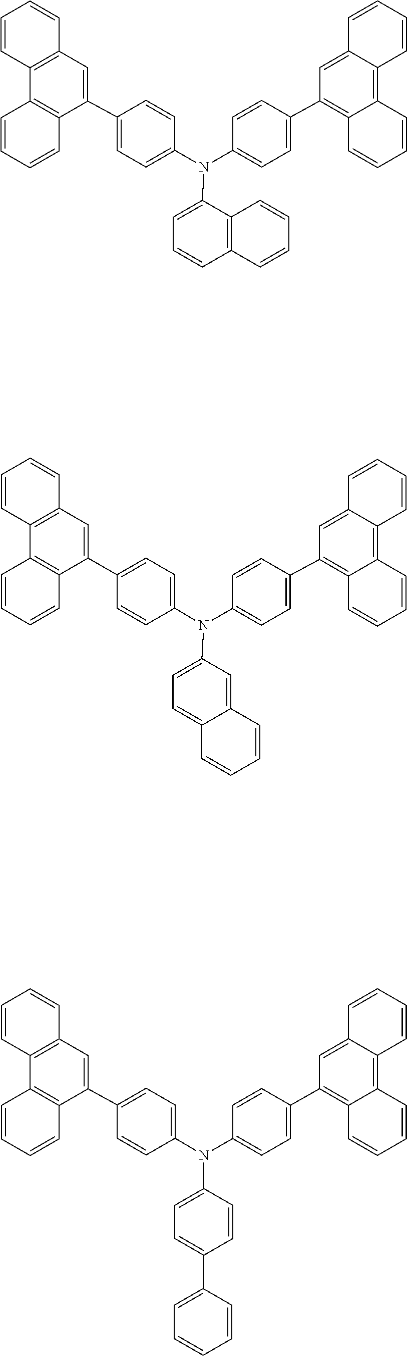 [SMILES: c1ccc2c(N(c3ccc(-c4cc5ccccc5c5ccccc45)cc3)c3ccc(-c4cc5ccccc5c5ccccc45)cc3)cccc2c1 ; c1ccc2cc(N(c3ccc(-c4cc5ccccc5c5ccccc45)cc3)c3ccc(-c4cc5ccccc5c5ccccc45)cc3)ccc2c1 ; c1ccc(-c2ccc(N(c3ccc(-c4cc5ccccc5c5ccccc45)cc3)c3ccc(-c4cc5ccccc5c5ccccc45)cc3)cc2)cc1] 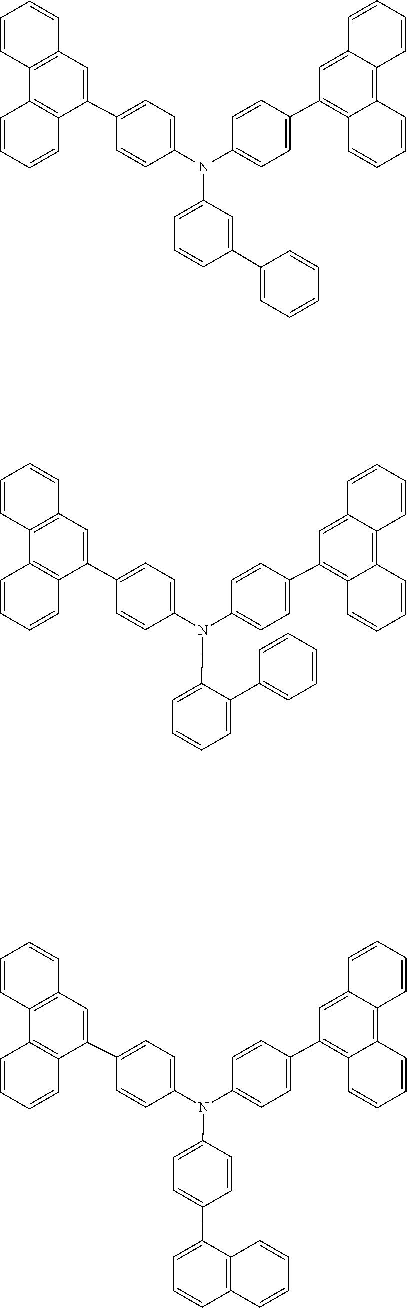 [SMILES: c1ccc(-c2cccc(N(c3ccc(-c4cc5ccccc5c5ccccc45)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cc4ccccc4c4ccccc34)cc2)cc1 ; c1ccc2c(-c3ccc(N(c4ccc(-c5cc6ccccc6c6ccccc56)cc4)c4ccc(-c5cc6ccccc6c6ccccc56)cc4)cc3)cccc2c1] 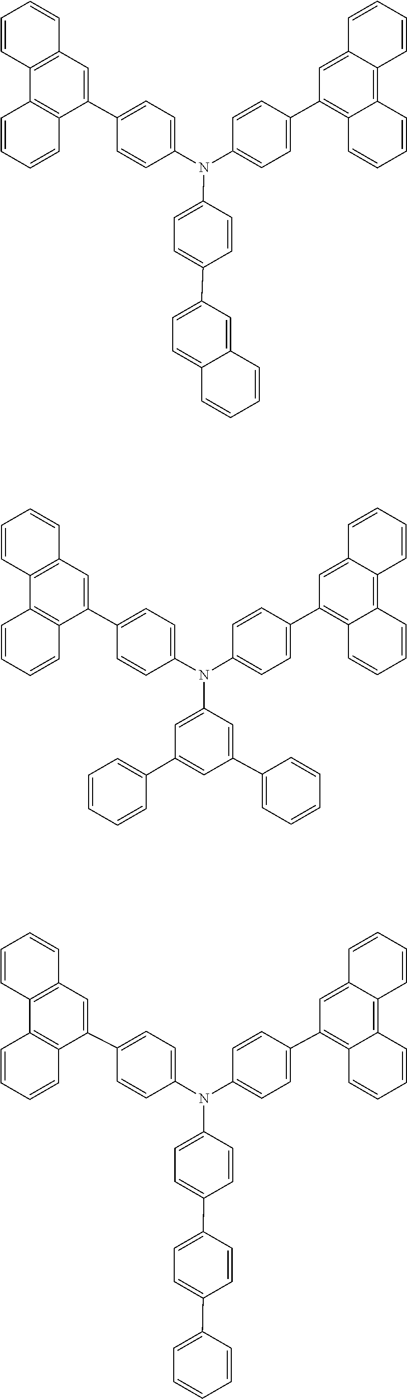 [SMILES: c1ccc2cc(-c3ccc(N(c4ccc(-c5cc6ccccc6c6ccccc56)cc4)c4ccc(-c5cc6ccccc6c6ccccc56)cc4)cc3)ccc2c1 ; c1ccc(-c2cc(-c3ccccc3)cc(N(c3ccc(-c4cc5ccccc5c5ccccc45)cc3)c3ccc(-c4cc5ccccc5c5ccccc45)cc3)c2)cc1 ; c1ccc(-c2ccc(-c3ccc(N(c4ccc(-c5cc6ccccc6c6ccccc56)cc4)c4ccc(-c5cc6ccccc6c6ccccc56)cc4)cc3)cc2)cc1] 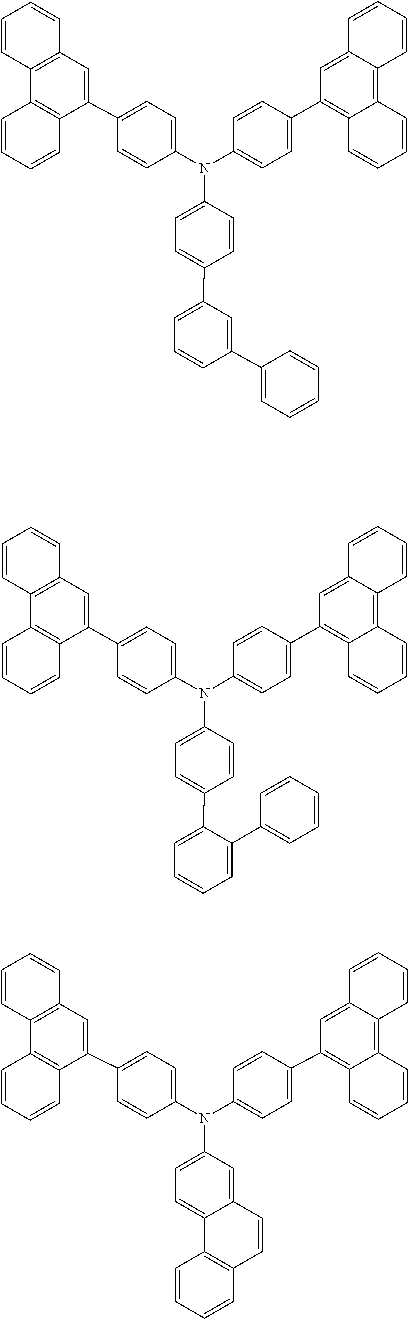 [SMILES: c1ccc(-c2cccc(-c3ccc(N(c4ccc(-c5cc6ccccc6c6ccccc56)cc4)c4ccc(-c5cc6ccccc6c6ccccc56)cc4)cc3)c2)cc1 ; c1ccc(-c2ccccc2-c2ccc(N(c3ccc(-c4cc5ccccc5c5ccccc45)cc3)c3ccc(-c4cc5ccccc5c5ccccc45)cc3)cc2)cc1 ; c1ccc2c(c1)ccc1cc(N(c3ccc(-c4cc5ccccc5c5ccccc45)cc3)c3ccc(-c4cc5ccccc5c5ccccc45)cc3)ccc12] 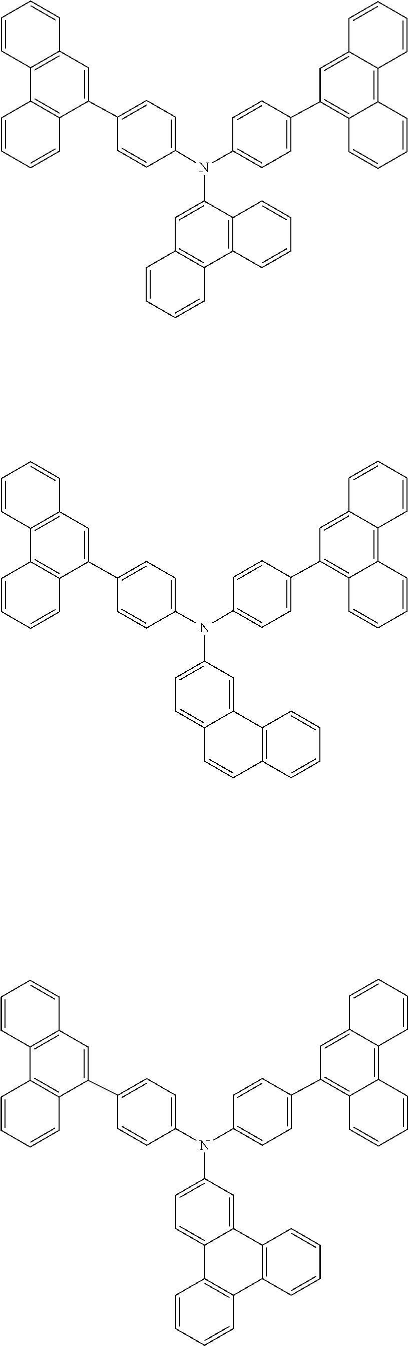 [SMILES: c1ccc2c(c1)cc(-c1ccc(N(c3ccc(-c4cc5ccccc5c5ccccc45)cc3)c3cc4ccccc4c4ccccc34)cc1)c1ccccc12 ; c1ccc2c(c1)ccc1ccc(N(c3ccc(-c4cc5ccccc5c5ccccc45)cc3)c3ccc(-c4cc5ccccc5c5ccccc45)cc3)cc12 ; c1ccc2c(c1)cc(-c1ccc(N(c3ccc(-c4cc5ccccc5c5ccccc45)cc3)c3ccc4c5ccccc5c5ccccc5c4c3)cc1)c1ccccc12] 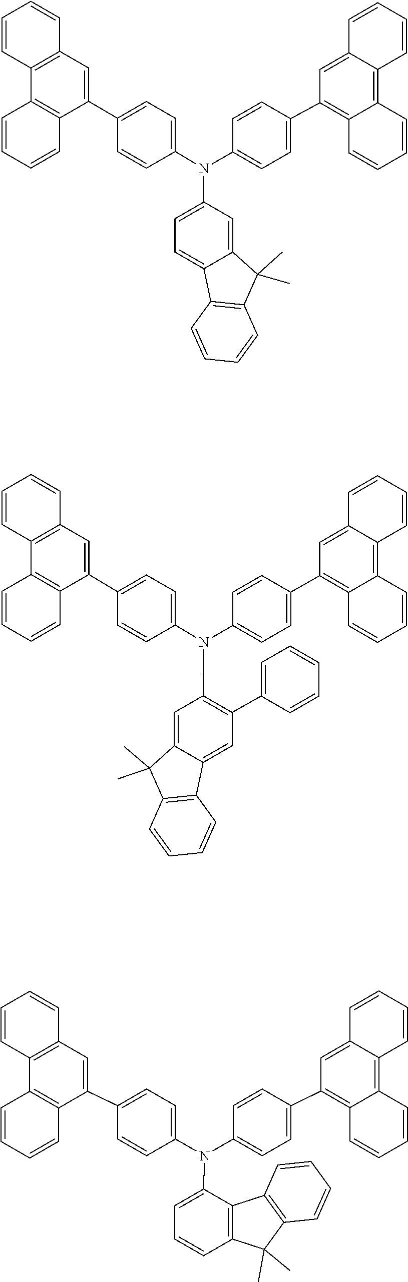 [SMILES: CC1(C)c2ccccc2-c2ccc(N(c3ccc(-c4cc5ccccc5c5ccccc45)cc3)c3ccc(-c4cc5ccccc5c5ccccc45)cc3)cc21 ; CC1(C)c2ccccc2-c2cc(-c3ccccc3)c(N(c3ccc(-c4cc5ccccc5c5ccccc45)cc3)c3ccc(-c4cc5ccccc5c5ccccc45)cc3)cc21 ; CC1(C)c2ccccc2-c2c(N(c3ccc(-c4cc5ccccc5c5ccccc45)cc3)c3ccc(-c4cc5ccccc5c5ccccc45)cc3)cccc21] 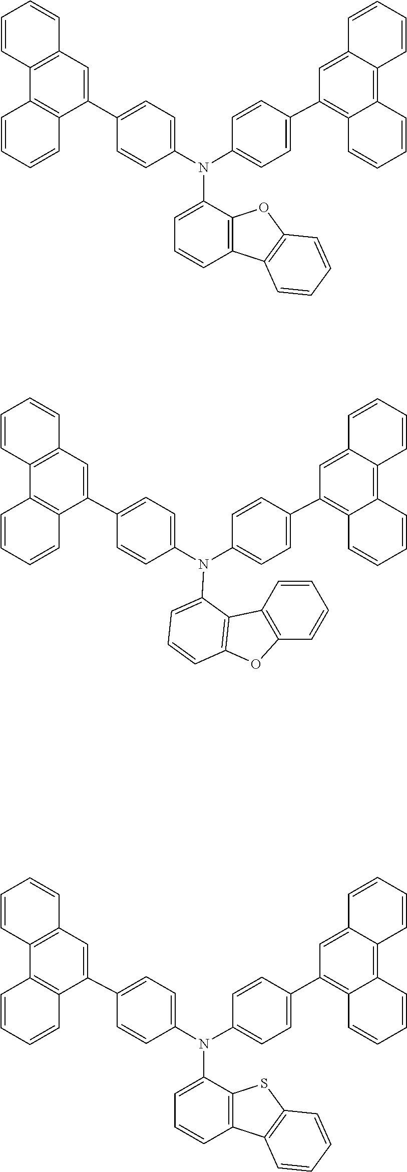 [SMILES: c1ccc2c(c1)cc(-c1ccc(N(c3ccc(-c4cc5ccccc5c5ccccc45)cc3)c3cccc4c3oc3ccccc34)cc1)c1ccccc12 ; c1ccc2c(c1)cc(-c1ccc(N(c3ccc(-c4cc5ccccc5c5ccccc45)cc3)c3cccc4oc5ccccc5c34)cc1)c1ccccc12 ; c1ccc2c(c1)cc(-c1ccc(N(c3ccc(-c4cc5ccccc5c5ccccc45)cc3)c3cccc4c3sc3ccccc34)cc1)c1ccccc12] 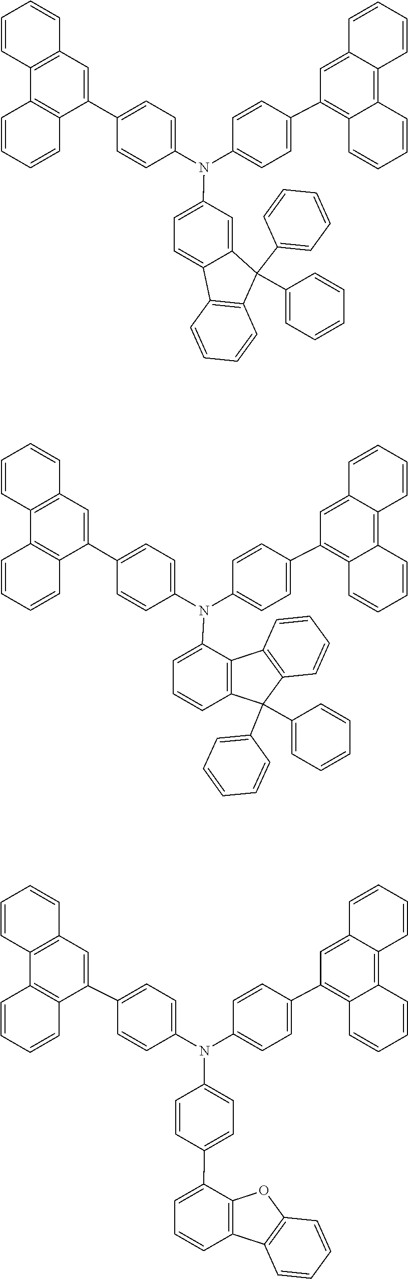 [SMILES: c1ccc(C2(c3ccccc3)c3ccccc3-c3ccc(N(c4ccc(-c5cc6ccccc6c6ccccc56)cc4)c4ccc(-c5cc6ccccc6c6ccccc56)cc4)cc32)cc1 ; c1ccc(C2(c3ccccc3)c3ccccc3-c3c(N(c4ccc(-c5cc6ccccc6c6ccccc56)cc4)c4ccc(-c5cc6ccccc6c6ccccc56)cc4)cccc32)cc1 ; c1ccc2c(c1)cc(-c1ccc(N(c3ccc(-c4cc5ccccc5c5ccccc45)cc3)c3ccc(-c4cccc5c4oc4ccccc45)cc3)cc1)c1ccccc12] 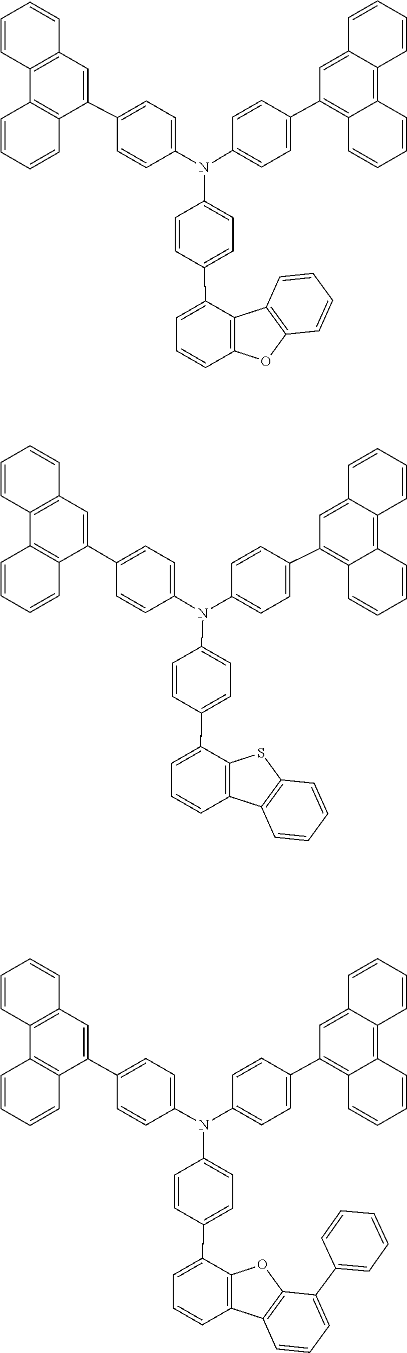 [SMILES: c1ccc2c(c1)cc(-c1ccc(N(c3ccc(-c4cc5ccccc5c5ccccc45)cc3)c3ccc(-c4cccc5oc6ccccc6c45)cc3)cc1)c1ccccc12 ; c1ccc2c(c1)cc(-c1ccc(N(c3ccc(-c4cc5ccccc5c5ccccc45)cc3)c3ccc(-c4cccc5c4sc4ccccc45)cc3)cc1)c1ccccc12 ; c1ccc(-c2cccc3c2oc2c(-c4ccc(N(c5ccc(-c6cc7ccccc7c7ccccc67)cc5)c5ccc(-c6cc7ccccc7c7ccccc67)cc5)cc4)cccc23)cc1] 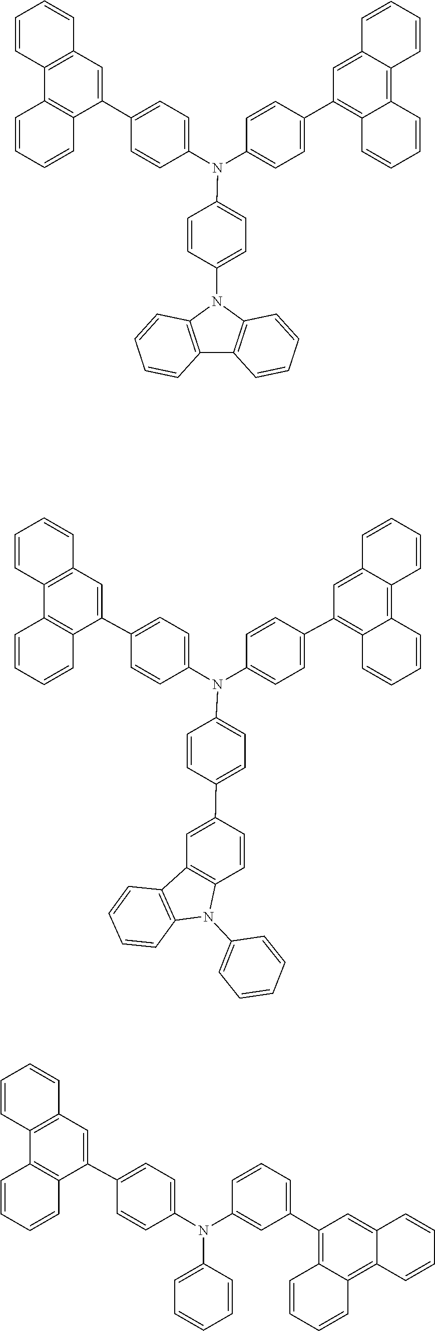 [SMILES: c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12 ; c1ccc(-n2c3ccccc3c3cc(-c4ccc(N(c5ccc(-c6cc7ccccc7c7ccccc67)cc5)c5ccc(-c6cc7ccccc7c7ccccc67)cc5)cc4)ccc32)cc1 ; c1ccc(N(c2ccc(-c3cc4ccccc4c4ccccc34)cc2)c2cccc(-c3cc4ccccc4c4ccccc34)c2)cc1] 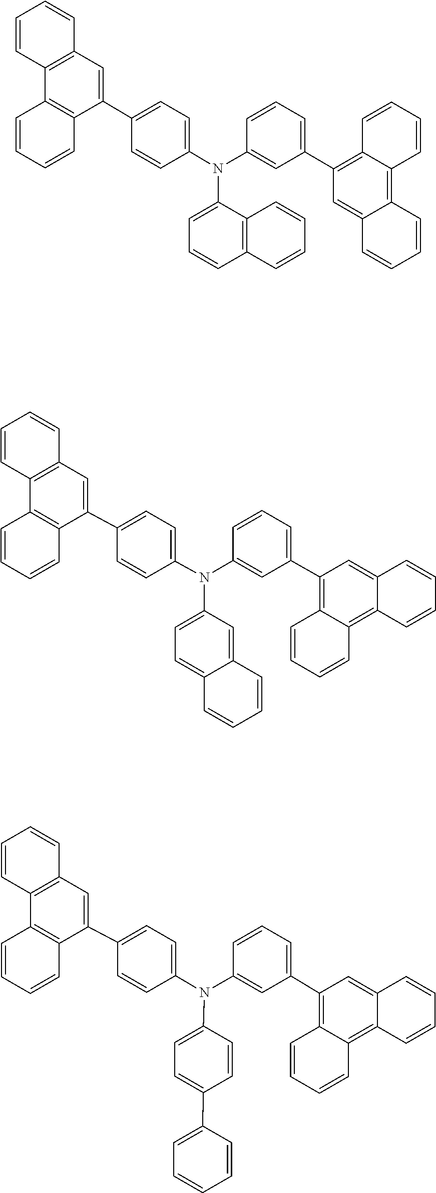 [SMILES: c1cc(-c2cc3ccccc3c3ccccc23)cc(N(c2ccc(-c3cc4ccccc4c4ccccc34)cc2)c2cccc3ccccc23)c1 ; c1cc(-c2cc3ccccc3c3ccccc23)cc(N(c2ccc(-c3cc4ccccc4c4ccccc34)cc2)c2ccc3ccccc3c2)c1 ; c1ccc(-c2ccc(N(c3ccc(-c4cc5ccccc5c5ccccc45)cc3)c3cccc(-c4cc5ccccc5c5ccccc45)c3)cc2)cc1] 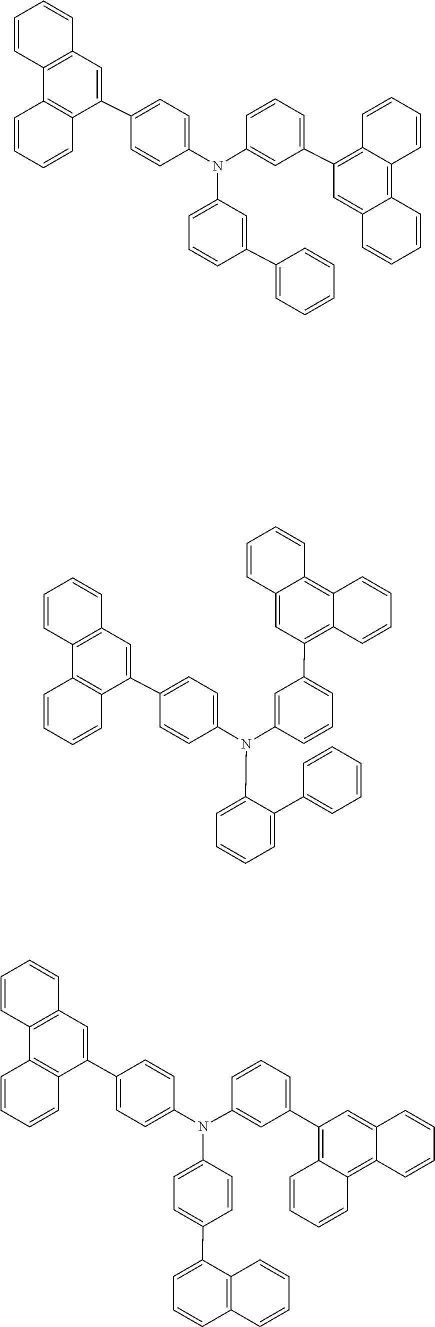 [SMILES: c1ccc(-c2cccc(N(c3ccc(-c4cc5ccccc5c5ccccc45)cc3)c3cccc(-c4cc5ccccc5c5ccccc45)c3)c2)cc1 ; c1ccc(-c2ccccc2N(c2ccc(-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3cccc4ccccc34)cc2)c2ccc(-c3cc4ccccc4c4ccccc34)cc2)c1] 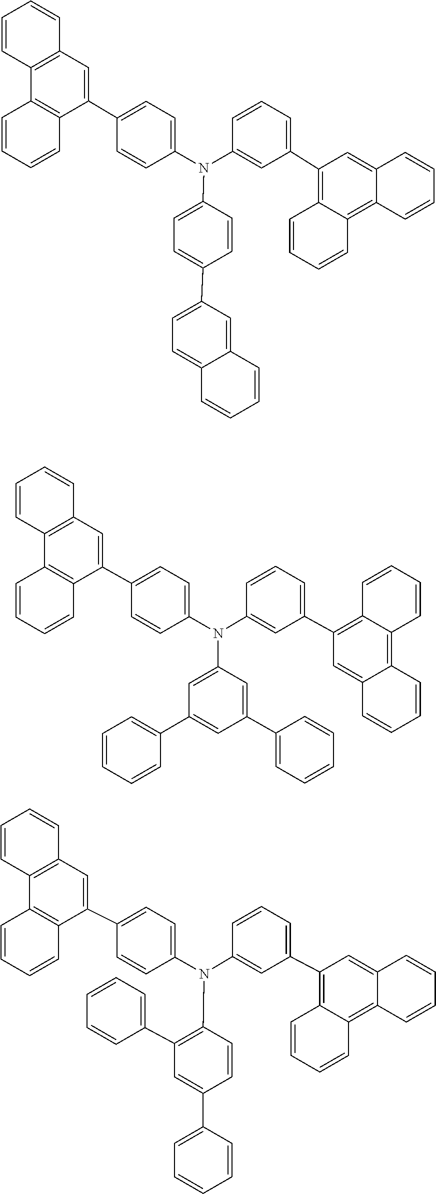 [SMILES: c1cc(-c2cc3ccccc3c3ccccc23)cc(N(c2ccc(-c3ccc4ccccc4c3)cc2)c2ccc(-c3cc4ccccc4c4ccccc34)cc2)c1 ; c1ccc(-c2cc(-c3ccccc3)cc(N(c3ccc(-c4cc5ccccc5c5ccccc45)cc3)c3cccc(-c4cc5ccccc5c5ccccc45)c3)c2)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1] 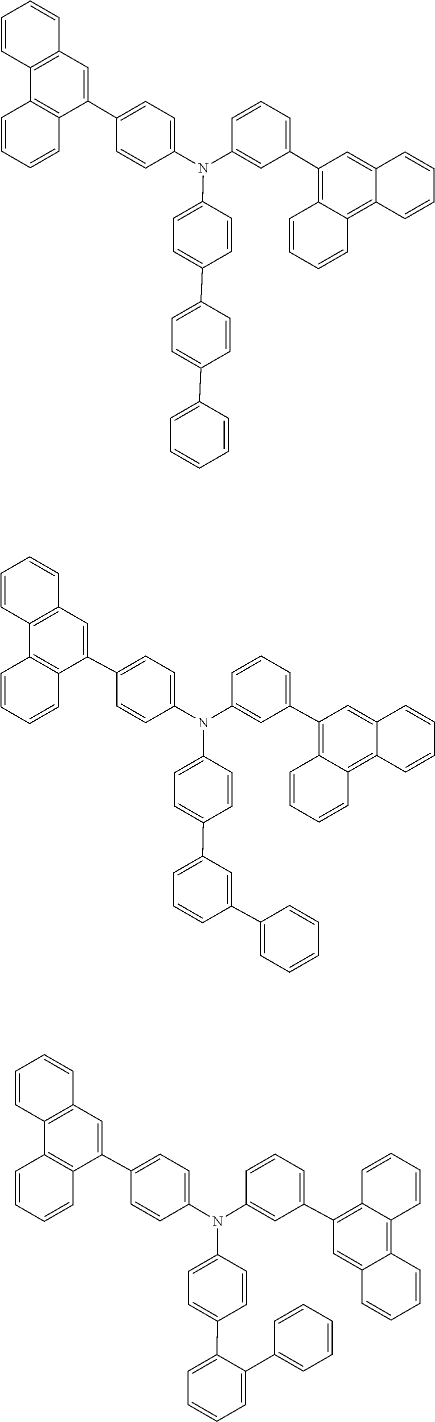 [SMILES: c1ccc(-c2ccc(-c3ccc(N(c4ccc(-c5cc6ccccc6c6ccccc56)cc4)c4cccc(-c5cc6ccccc6c6ccccc56)c4)cc3)cc2)cc1 ; c1ccc(-c2cccc(-c3ccc(N(c4ccc(-c5cc6ccccc6c6ccccc56)cc4)c4cccc(-c5cc6ccccc6c6ccccc56)c4)cc3)c2)cc1 ; c1ccc(-c2ccccc2-c2ccc(N(c3ccc(-c4cc5ccccc5c5ccccc45)cc3)c3cccc(-c4cc5ccccc5c5ccccc45)c3)cc2)cc1] 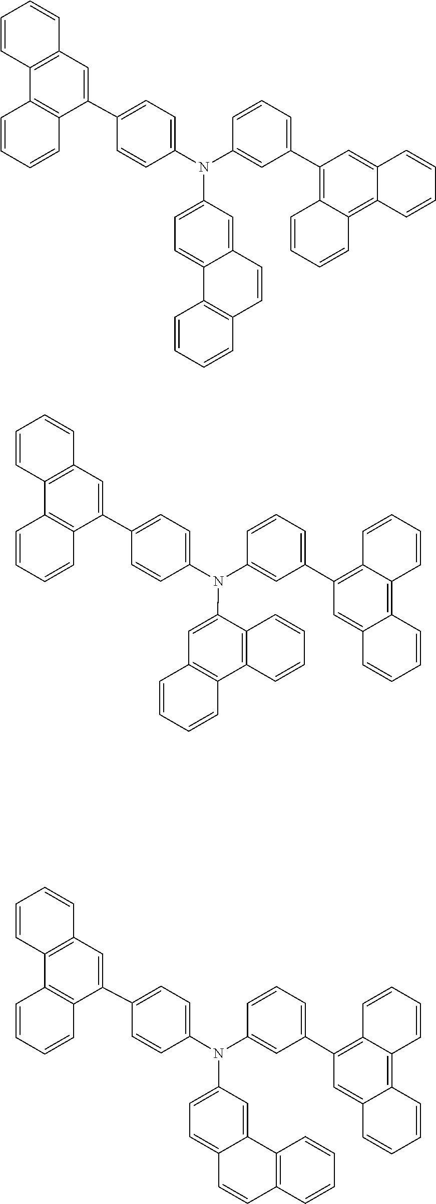 [SMILES: c1cc(-c2cc3ccccc3c3ccccc23)cc(N(c2ccc(-c3cc4ccccc4c4ccccc34)cc2)c2ccc3c(ccc4ccccc43)c2)c1 ; c1cc(-c2cc3ccccc3c3ccccc23)cc(N(c2ccc(-c3cc4ccccc4c4ccccc34)cc2)c2cc3ccccc3c3ccccc23)c1 ; c1cc(-c2cc3ccccc3c3ccccc23)cc(N(c2ccc(-c3cc4ccccc4c4ccccc34)cc2)c2ccc3ccc4ccccc4c3c2)c1] 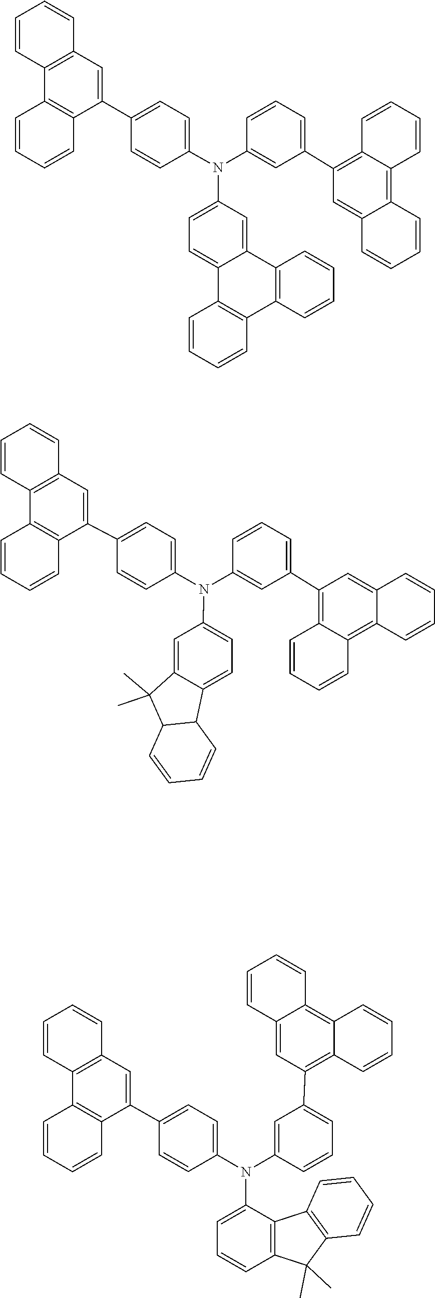 [SMILES: c1cc(-c2cc3ccccc3c3ccccc23)cc(N(c2ccc(-c3cc4ccccc4c4ccccc34)cc2)c2ccc3c4ccccc4c4ccccc4c3c2)c1 ; CC1(C)c2cc(N(c3ccc(-c4cc5ccccc5c5ccccc45)cc3)c3cccc(-c4cc5ccccc5c5ccccc45)c3)ccc2C2C=CC=CC21 ; CC1(C)c2ccccc2-c2c(N(c3ccc(-c4cc5ccccc5c5ccccc45)cc3)c3cccc(-c4cc5ccccc5c5ccccc45)c3)cccc21] 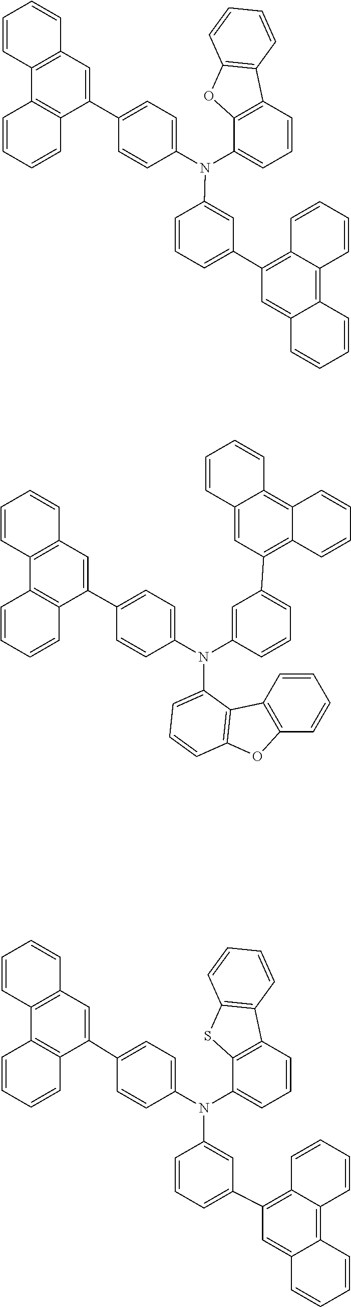 [SMILES: c1cc(-c2cc3ccccc3c3ccccc23)cc(N(c2ccc(-c3cc4ccccc4c4ccccc34)cc2)c2cccc3c2oc2ccccc23)c1 ; c1cc(-c2cc3ccccc3c3ccccc23)cc(N(c2ccc(-c3cc4ccccc4c4ccccc34)cc2)c2cccc3oc4ccccc4c23)c1 ; c1cc(-c2cc3ccccc3c3ccccc23)cc(N(c2ccc(-c3cc4ccccc4c4ccccc34)cc2)c2cccc3c2sc2ccccc23)c1] 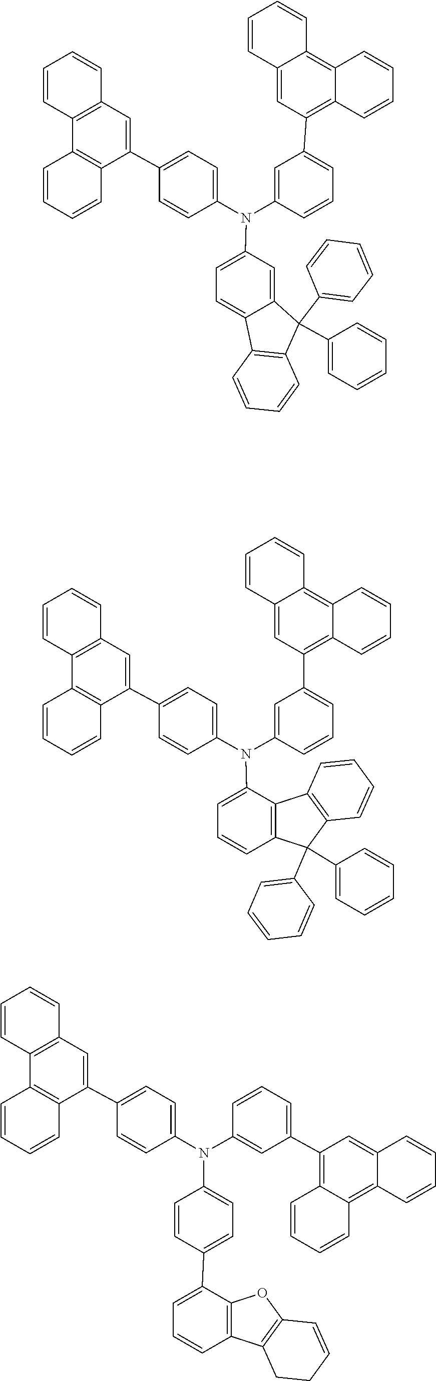 [SMILES: c1ccc(C2(c3ccccc3)c3ccccc3-c3ccc(N(c4ccc(-c5cc6ccccc6c6ccccc56)cc4)c4cccc(-c5cc6ccccc6c6ccccc56)c4)cc32)cc1 ; c1ccc(C2(c3ccccc3)c3ccccc3-c3c(N(c4ccc(-c5cc6ccccc6c6ccccc56)cc4)c4cccc(-c5cc6ccccc6c6ccccc56)c4)cccc32)cc1 ; C1=Cc2oc3c(-c4ccc(N(c5ccc(-c6cc7ccccc7c7ccccc67)cc5)c5cccc(-c6cc7ccccc7c7ccccc67)c5)cc4)cccc3c2CC1] 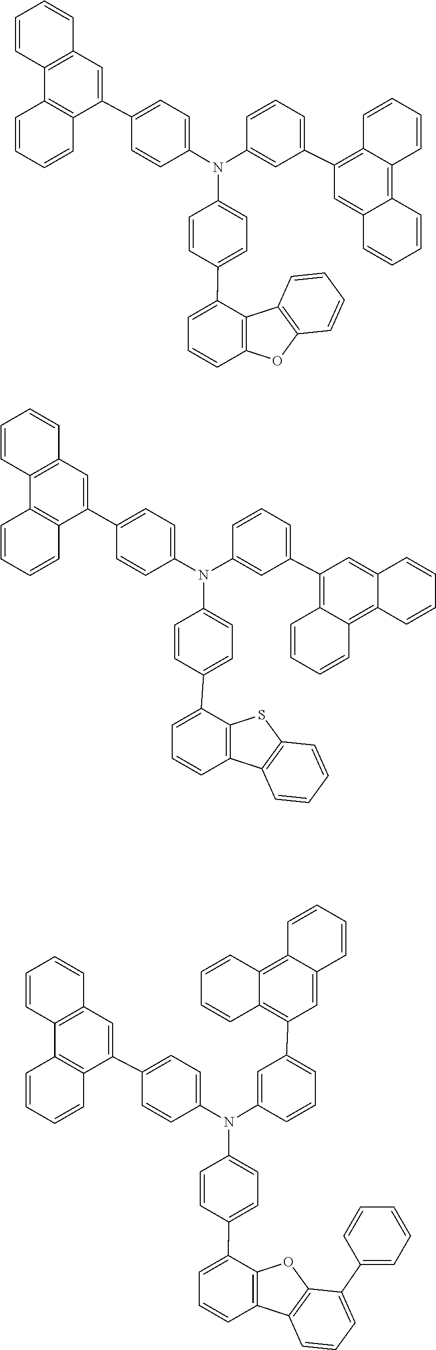 [SMILES: c1cc(-c2cc3ccccc3c3ccccc23)cc(N(c2ccc(-c3cc4ccccc4c4ccccc34)cc2)c2ccc(-c3cccc4oc5ccccc5c34)cc2)c1 ; c1cc(-c2cc3ccccc3c3ccccc23)cc(N(c2ccc(-c3cc4ccccc4c4ccccc34)cc2)c2ccc(-c3cccc4c3sc3ccccc34)cc2)c1 ; c1ccc(-c2cccc3c2oc2c(-c4ccc(N(c5ccc(-c6cc7ccccc7c7ccccc67)cc5)c5cccc(-c6cc7ccccc7c7ccccc67)c5)cc4)cccc23)cc1] 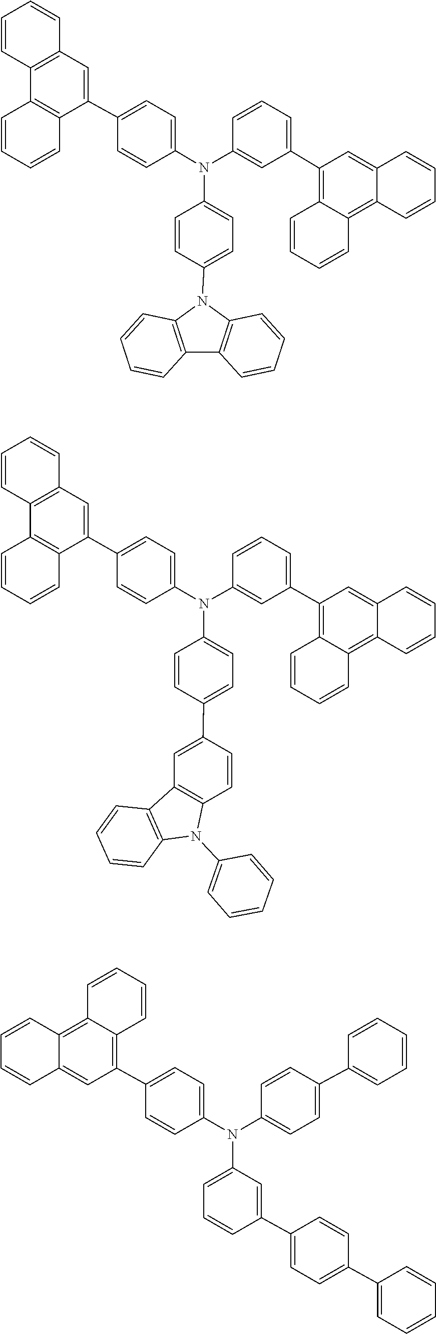 [SMILES: c1cc(-c2cc3ccccc3c3ccccc23)cc(N(c2ccc(-c3cc4ccccc4c4ccccc34)cc2)c2ccc(-n3c4ccccc4c4ccccc43)cc2)c1 ; c1ccc(-n2c3ccccc3c3cc(-c4ccc(N(c5ccc(-c6cc7ccccc7c7ccccc67)cc5)c5cccc(-c6cc7ccccc7c7ccccc67)c5)cc4)ccc32)cc1 ; c1ccc(-c2ccc(-c3cccc(N(c4ccc(-c5ccccc5)cc4)c4ccc(-c5cc6ccccc6c6ccccc56)cc4)c3)cc2)cc1] 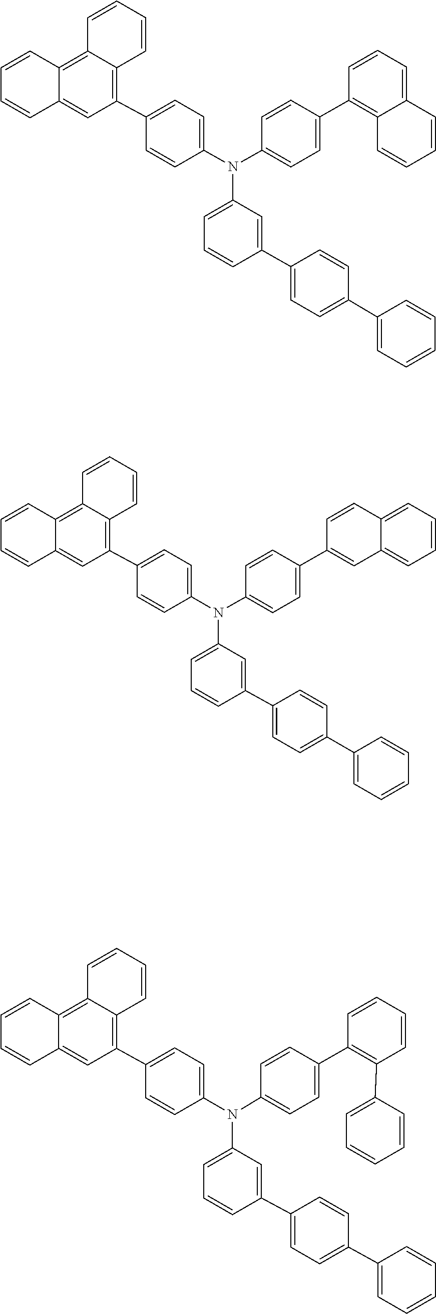 [SMILES: c1ccc(-c2ccc(-c3cccc(N(c4ccc(-c5cccc6ccccc56)cc4)c4ccc(-c5cc6ccccc6c6ccccc56)cc4)c3)cc2)cc1 ; c1ccc(-c2ccc(-c3cccc(N(c4ccc(-c5ccc6ccccc6c5)cc4)c4ccc(-c5cc6ccccc6c6ccccc56)cc4)c3)cc2)cc1 ; c1ccc(-c2ccc(-c3cccc(N(c4ccc(-c5ccccc5-c5ccccc5)cc4)c4ccc(-c5cc6ccccc6c6ccccc56)cc4)c3)cc2)cc1] 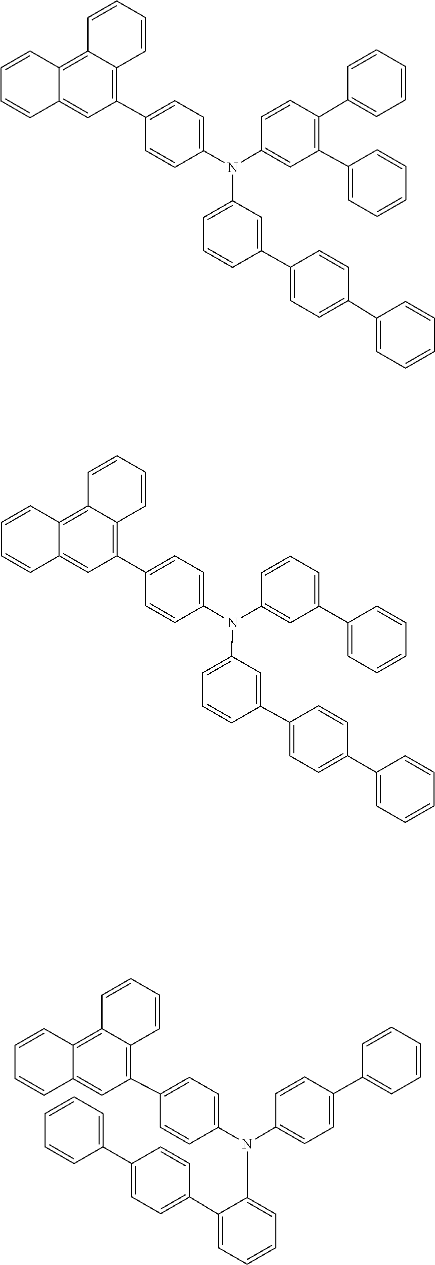 [SMILES: c1ccc(-c2ccc(-c3cccc(N(c4ccc(-c5cc6ccccc6c6ccccc56)cc4)c4ccc(-c5ccccc5)c(-c5ccccc5)c4)c3)cc2)cc1 ; c1ccc(-c2ccc(-c3cccc(N(c4ccc(-c5cc6ccccc6c6ccccc56)cc4)c4cccc(-c5ccccc5)c4)c3)cc2)cc1 ; c1ccc(-c2ccc(-c3ccccc3N(c3ccc(-c4ccccc4)cc3)c3ccc(-c4cc5ccccc5c5ccccc45)cc3)cc2)cc1] 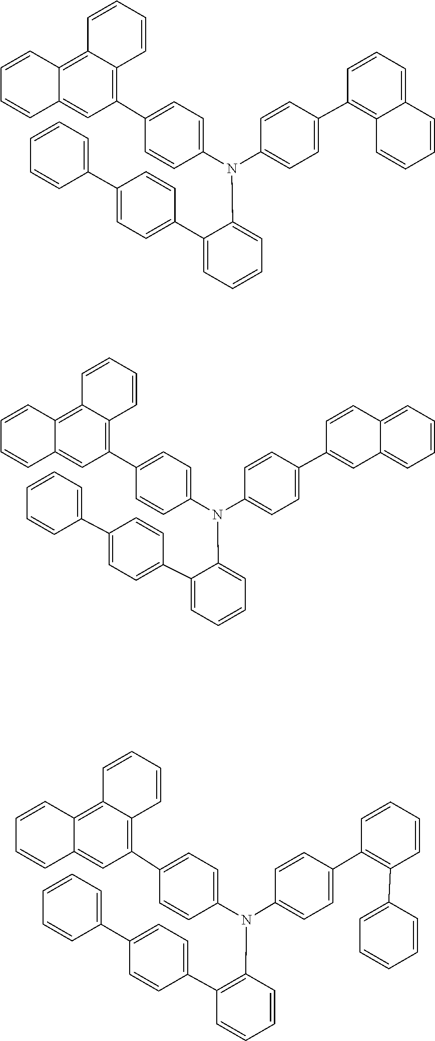 [SMILES: c1ccc(-c2ccc(-c3ccccc3N(c3ccc(-c4cccc5ccccc45)cc3)c3ccc(-c4cc5ccccc5c5ccccc45)cc3)cc2)cc1 ; c1ccc(-c2ccc(-c3ccccc3N(c3ccc(-c4ccc5ccccc5c4)cc3)c3ccc(-c4cc5ccccc5c5ccccc45)cc3)cc2)cc1 ; c1ccc(-c2ccc(-c3ccccc3N(c3ccc(-c4ccccc4-c4ccccc4)cc3)c3ccc(-c4cc5ccccc5c5ccccc45)cc3)cc2)cc1] 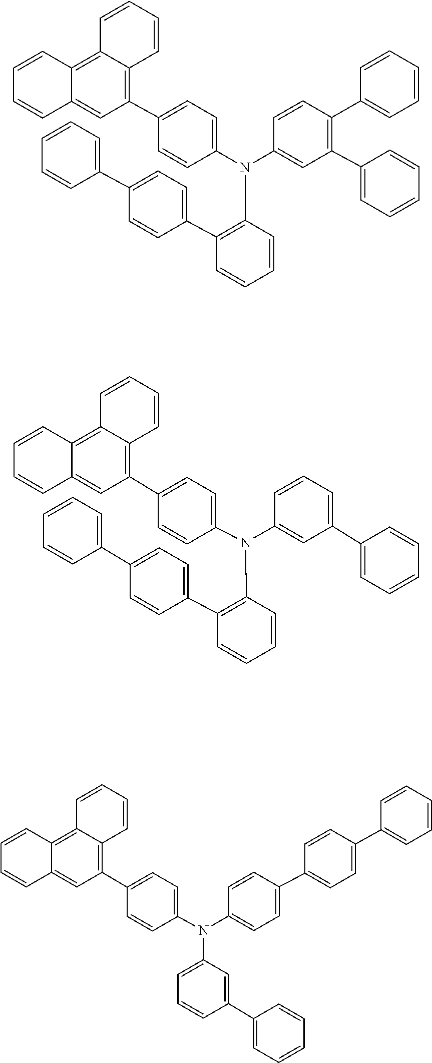 [SMILES: c1ccc(-c2ccc(-c3ccccc3N(c3ccc(-c4cc5ccccc5c5ccccc45)cc3)c3ccc(-c4ccccc4)c(-c4ccccc4)c3)cc2)cc1 ; c1ccc(-c2ccc(-c3ccccc3N(c3ccc(-c4cc5ccccc5c5ccccc45)cc3)c3cccc(-c4ccccc4)c3)cc2)cc1 ; c1ccc(-c2ccc(-c3ccc(N(c4ccc(-c5cc6ccccc6c6ccccc56)cc4)c4cccc(-c5ccccc5)c4)cc3)cc2)cc1] 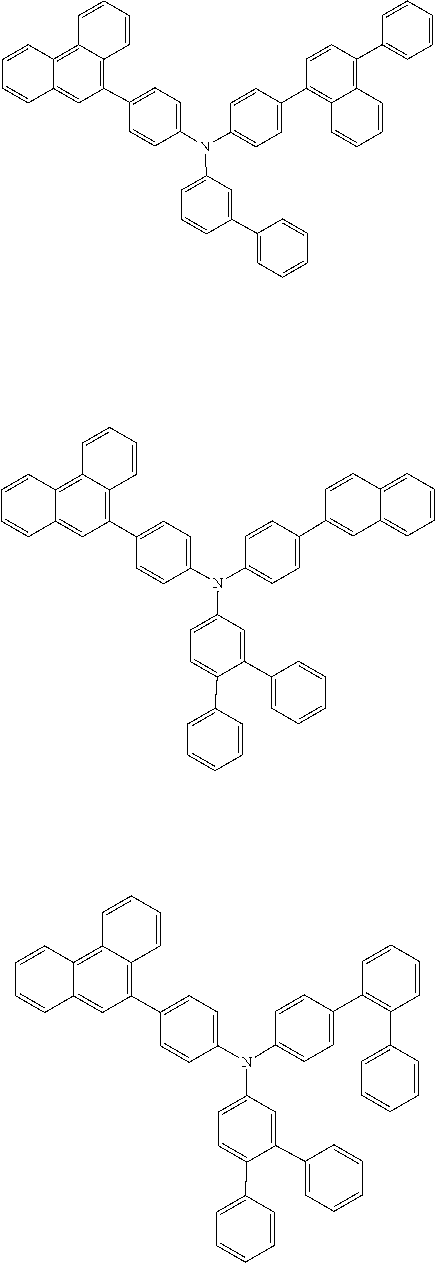 [SMILES: c1ccc(-c2cccc(N(c3ccc(-c4ccc(-c5ccccc5)c5ccccc45)cc3)c3ccc(-c4cc5ccccc5c5ccccc45)cc3)c2)cc1 ; c1ccc(-c2ccc(N(c3ccc(-c4ccc5ccccc5c4)cc3)c3ccc(-c4cc5ccccc5c5ccccc45)cc3)cc2-c2ccccc2)cc1 ; c1ccc(-c2ccccc2-c2ccc(N(c3ccc(-c4cc5ccccc5c5ccccc45)cc3)c3ccc(-c4ccccc4)c(-c4ccccc4)c3)cc2)cc1] 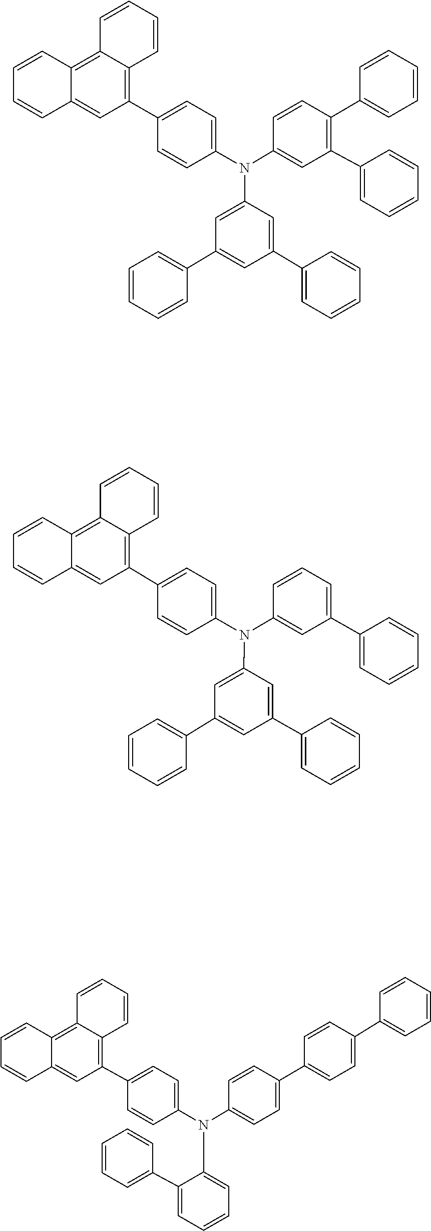 [SMILES: c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2ccc(-c3ccc(N(c4ccc(-c5cc6ccccc6c6ccccc56)cc4)c4ccccc4-c4ccccc4)cc3)cc2)cc1] 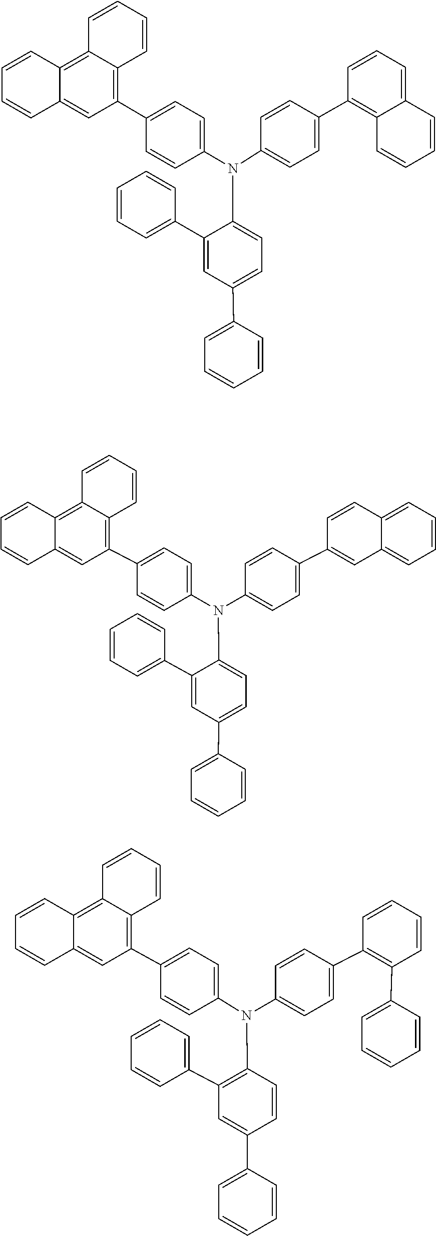 [SMILES: c1ccc(-c2ccc(N(c3ccc(-c4cccc5ccccc45)cc3)c3ccc(-c4cc5ccccc5c5ccccc45)cc3)c(-c3ccccc3)c2)cc1 ; c1ccc(-c2ccc(N(c3ccc(-c4ccc5ccccc5c4)cc3)c3ccc(-c4cc5ccccc5c5ccccc45)cc3)c(-c3ccccc3)c2)cc1 ; c1ccc(-c2ccc(N(c3ccc(-c4ccccc4-c4ccccc4)cc3)c3ccc(-c4cc5ccccc5c5ccccc45)cc3)c(-c3ccccc3)c2)cc1] 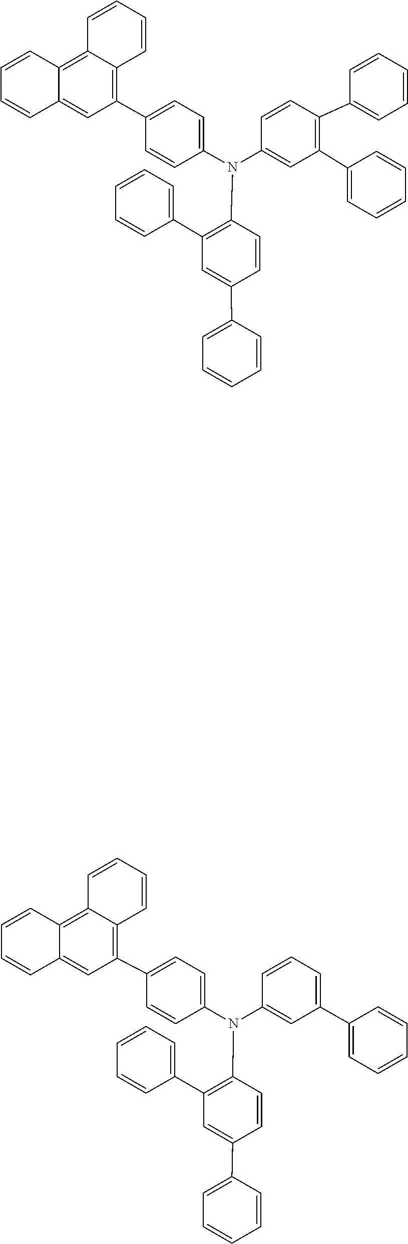 [SMILES: c1ccc(-c2ccc(N(c3ccc(-c4cc5ccccc5c5ccccc45)cc3)c3ccc(-c4ccccc4)c(-c4ccccc4)c3)c(-c3ccccc3)c2)cc1 ; c1ccc(-c2cccc(N(c3ccc(-c4cc5ccccc5c5ccccc45)cc3)c3ccc(-c4ccccc4)cc3-c3ccccc3)c2)cc1] 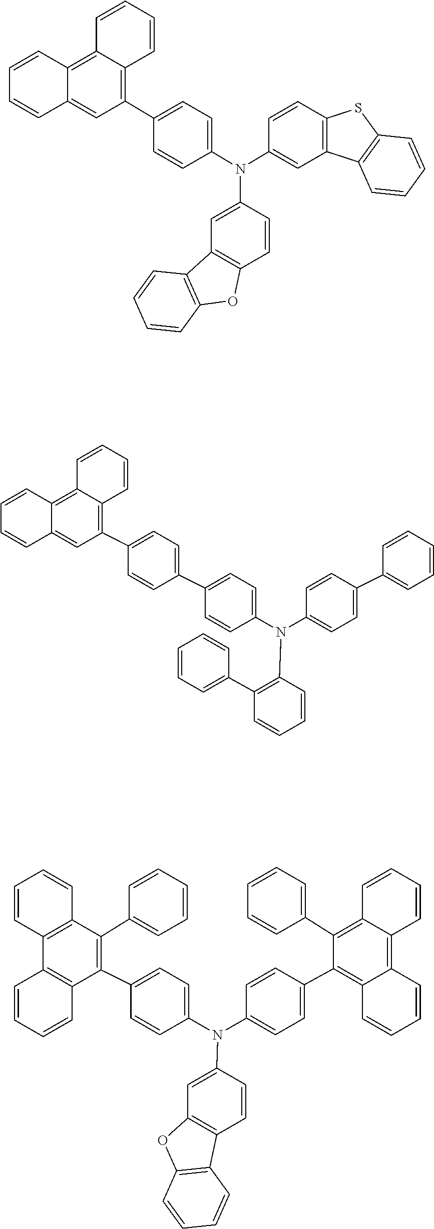 [SMILES: c1ccc2c(c1)cc(-c1ccc(N(c3ccc4oc5ccccc5c4c3)c3ccc4sc5ccccc5c4c3)cc1)c1ccccc12 ; c1ccc(-c2ccc(N(c3ccc(-c4ccc(-c5cc6ccccc6c6ccccc56)cc4)cc3)c3ccccc3-c3ccccc3)cc2)cc1 ; c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1] 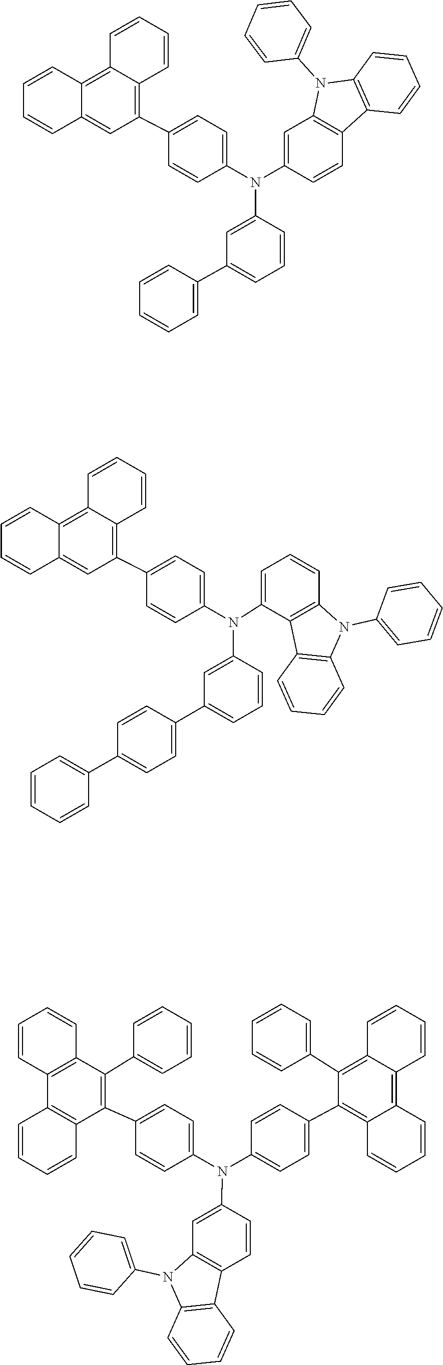 [SMILES: c1ccc(-c2cccc(N(c3ccc(-c4cc5ccccc5c5ccccc45)cc3)c3ccc4c5ccccc5n(-c5ccccc5)c4c3)c2)cc1 ; c1ccc(-c2ccc(-c3cccc(N(c4ccc(-c5cc6ccccc6c6ccccc56)cc4)c4cccc5c4c4ccccc4n5-c4ccccc4)c3)cc2)cc1 ; c1ccc(-c2c(-c3ccc(N(c4ccc(-c5c(-c6ccccc6)c6ccccc6c6ccccc56)cc4)c4ccc5c6ccccc6n(-c6ccccc6)c5c4)cc3)c3ccccc3c3ccccc23)cc1] 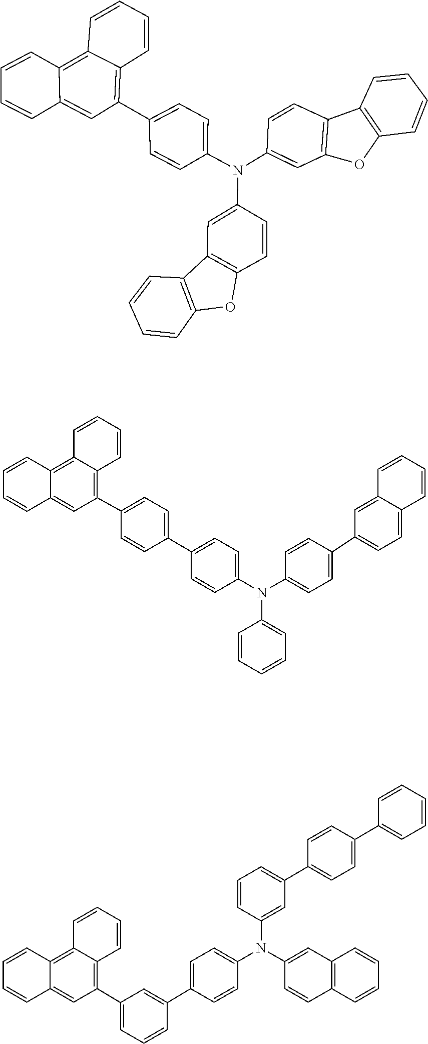 [SMILES: c1ccc2c(c1)cc(-c1ccc(N(c3ccc4c(c3)oc3ccccc34)c3ccc4oc5ccccc5c4c3)cc1)c1ccccc12 ; c1ccc(N(c2ccc(-c3ccc(-c4cc5ccccc5c5ccccc45)cc3)cc2)c2ccc(-c3ccc4ccccc4c3)cc2)cc1 ; c1ccc(-c2ccc(-c3cccc(N(c4ccc(-c5cccc(-c6cc7ccccc7c7ccccc67)c5)cc4)c4ccc5ccccc5c4)c3)cc2)cc1] 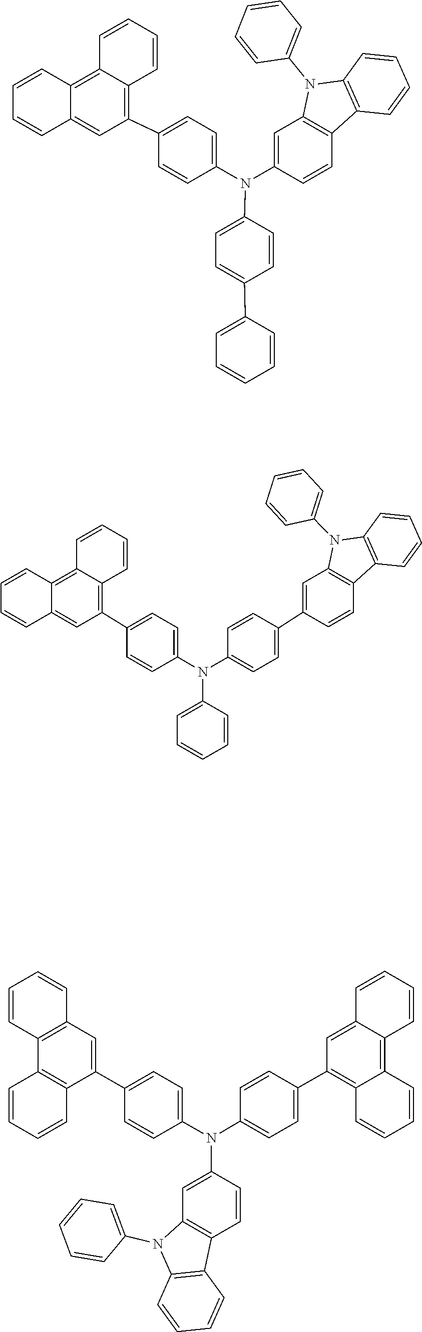 [SMILES: c1ccc(-c2ccc(N(c3ccc(-c4cc5ccccc5c5ccccc45)cc3)c3ccc4c5ccccc5n(-c5ccccc5)c4c3)cc2)cc1 ; c1ccc(N(c2ccc(-c3ccc4c5ccccc5n(-c5ccccc5)c4c3)cc2)c2ccc(-c3cc4ccccc4c4ccccc34)cc2)cc1 ; c1ccc(-n2c3ccccc3c3ccc(N(c4ccc(-c5cc6ccccc6c6ccccc56)cc4)c4ccc(-c5cc6ccccc6c6ccccc56)cc4)cc32)cc1] 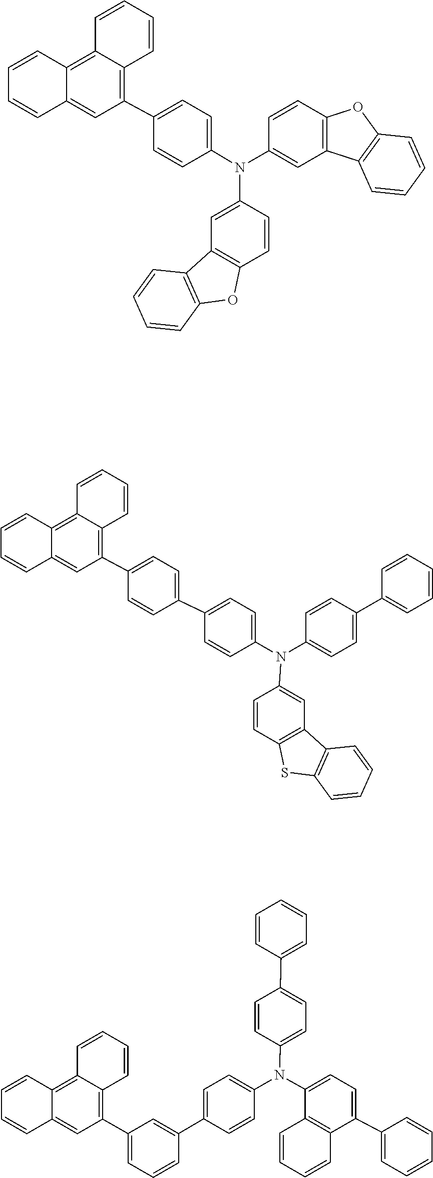 [SMILES: c1ccc2c(c1)cc(-c1ccc(N(c3ccc4oc5ccccc5c4c3)c3ccc4oc5ccccc5c4c3)cc1)c1ccccc12 ; c1ccc(-c2ccc(N(c3ccc(-c4ccc(-c5cc6ccccc6c6ccccc56)cc4)cc3)c3ccc4sc5ccccc5c4c3)cc2)cc1 ; c1ccc(-c2ccc(N(c3ccc(-c4cccc(-c5cc6ccccc6c6ccccc56)c4)cc3)c3ccc(-c4ccccc4)c4ccccc34)cc2)cc1] 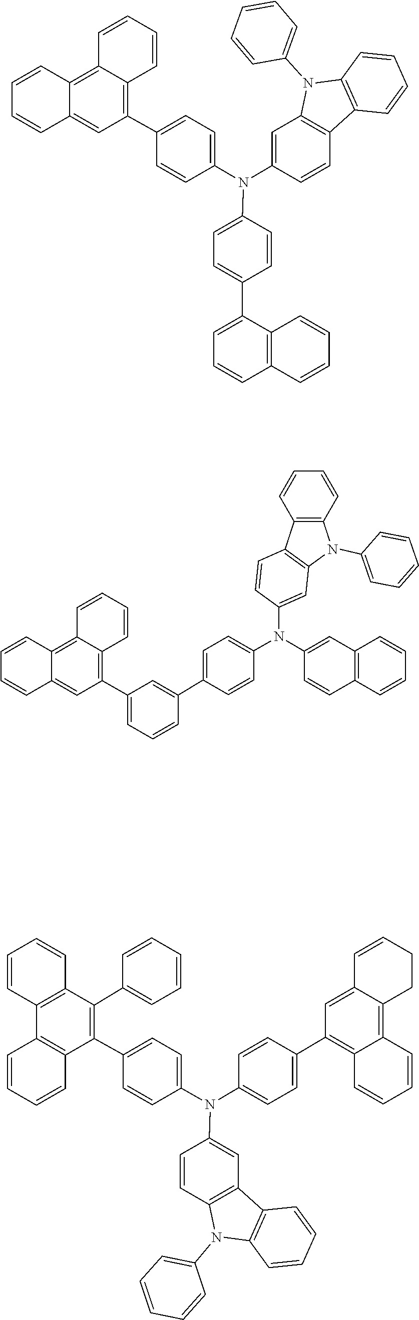 [SMILES: c1ccc(-n2c3ccccc3c3ccc(N(c4ccc(-c5cccc6ccccc56)cc4)c4ccc(-c5cc6ccccc6c6ccccc56)cc4)cc32)cc1 ; c1ccc(-n2c3ccccc3c3ccc(N(c4ccc(-c5cccc(-c6cc7ccccc7c7ccccc67)c5)cc4)c4ccc5ccccc5c4)cc32)cc1 ; C1=Cc2cc(-c3ccc(N(c4ccc(-c5c(-c6ccccc6)c6ccccc6c6ccccc56)cc4)c4ccc5c(c4)c4ccccc4n5-c4ccccc4)cc3)c3ccccc3c2CC1] 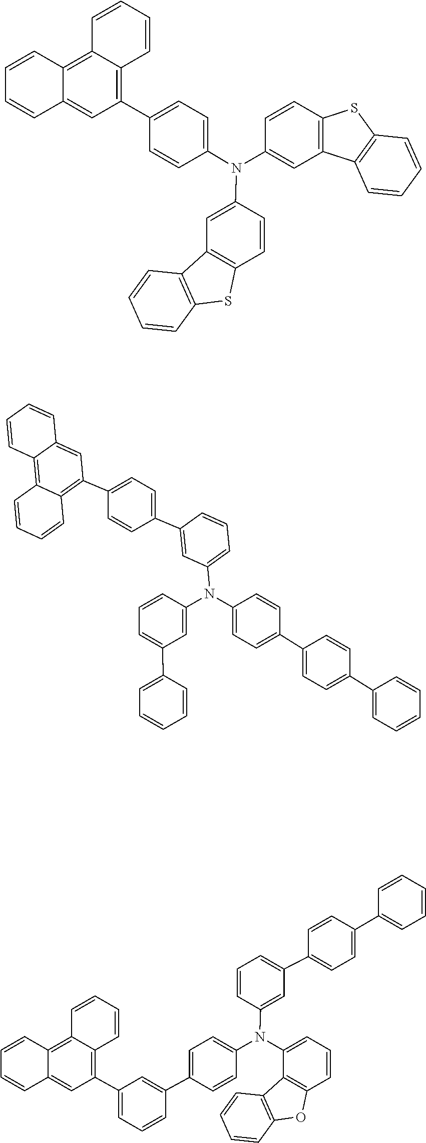 [SMILES: c1ccc2c(c1)cc(-c1ccc(N(c3ccc4sc5ccccc5c4c3)c3ccc4sc5ccccc5c4c3)cc1)c1ccccc12 ; c1ccc(-c2ccc(-c3ccc(N(c4cccc(-c5ccccc5)c4)c4cccc(-c5ccc(-c6cc7ccccc7c7ccccc67)cc5)c4)cc3)cc2)cc1 ; c1ccc(-c2ccc(-c3cccc(N(c4ccc(-c5cccc(-c6cc7ccccc7c7ccccc67)c5)cc4)c4cccc5oc6ccccc6c45)c3)cc2)cc1] 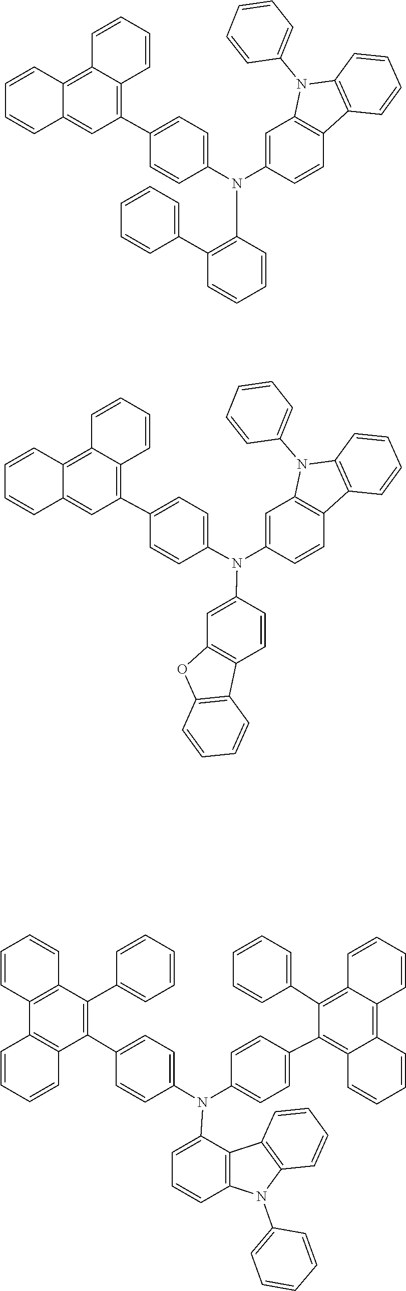 [SMILES: c1ccc(-c2ccccc2N(c2ccc(-c3cc4ccccc4c4ccccc34)cc2)c2ccc3c4ccccc4n(-c4ccccc4)c3c2)cc1 ; c1ccc(-n2c3ccccc3c3ccc(N(c4ccc(-c5cc6ccccc6c6ccccc56)cc4)c4ccc5c(c4)oc4ccccc45)cc32)cc1 ; c1ccc(-c2c(-c3ccc(N(c4ccc(-c5c(-c6ccccc6)c6ccccc6c6ccccc56)cc4)c4cccc5c4c4ccccc4n5-c4ccccc4)cc3)c3ccccc3c3ccccc23)cc1] 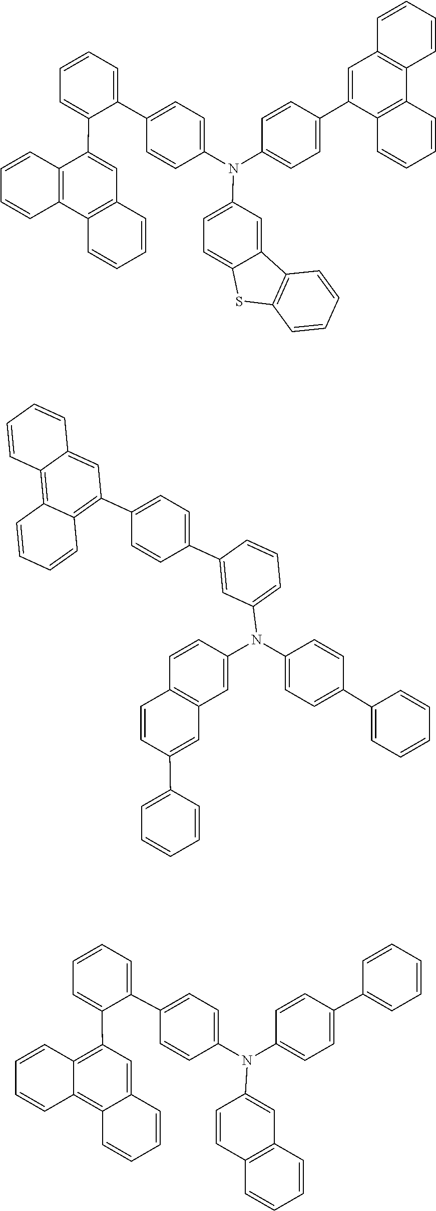 [SMILES: c1ccc(-c2cc3ccccc3c3ccccc23)c(-c2ccc(N(c3ccc(-c4cc5ccccc5c5ccccc45)cc3)c3ccc4sc5ccccc5c4c3)cc2)c1 ; c1ccc(-c2ccc(N(c3cccc(-c4ccc(-c5cc6ccccc6c6ccccc56)cc4)c3)c3ccc4ccc(-c5ccccc5)cc4c3)cc2)cc1 ; c1ccc(-c2ccc(N(c3ccc(-c4ccccc4-c4cc5ccccc5c5ccccc45)cc3)c3ccc4ccccc4c3)cc2)cc1] 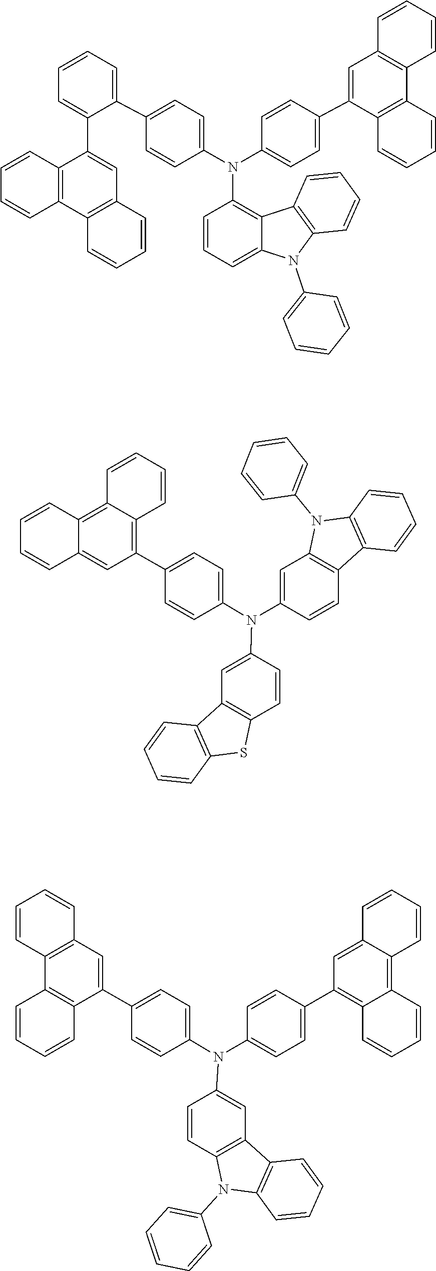 [SMILES: c1ccc(-n2c3ccccc3c3c(N(c4ccc(-c5ccccc5-c5cc6ccccc6c6ccccc56)cc4)c4ccc(-c5cc6ccccc6c6ccccc56)cc4)cccc32)cc1 ; c1ccc(-n2c3ccccc3c3ccc(N(c4ccc(-c5cc6ccccc6c6ccccc56)cc4)c4ccc5sc6ccccc6c5c4)cc32)cc1 ; c1ccc(-n2c3ccccc3c3cc(N(c4ccc(-c5cc6ccccc6c6ccccc56)cc4)c4ccc(-c5cc6ccccc6c6ccccc56)cc4)ccc32)cc1] 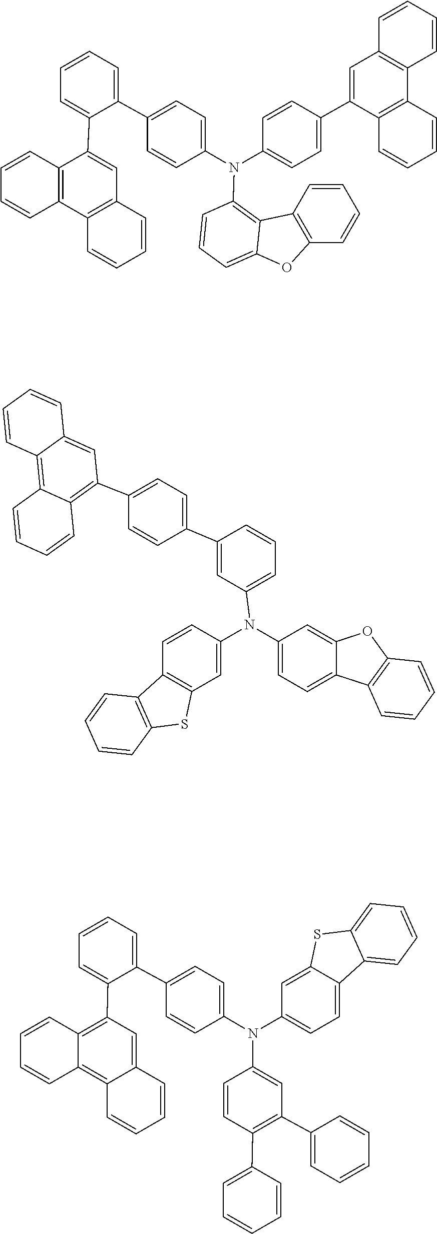 [SMILES: c1ccc(-c2cc3ccccc3c3ccccc23)c(-c2ccc(N(c3ccc(-c4cc5ccccc5c5ccccc45)cc3)c3cccc4oc5ccccc5c34)cc2)c1 ; c1cc(-c2ccc(-c3cc4ccccc4c4ccccc34)cc2)cc(N(c2ccc3c(c2)oc2ccccc23)c2ccc3c(c2)sc2ccccc23)c1 ; c1ccc(-c2ccc(N(c3ccc(-c4ccccc4-c4cc5ccccc5c5ccccc45)cc3)c3ccc4c(c3)sc3ccccc34)cc2-c2ccccc2)cc1] 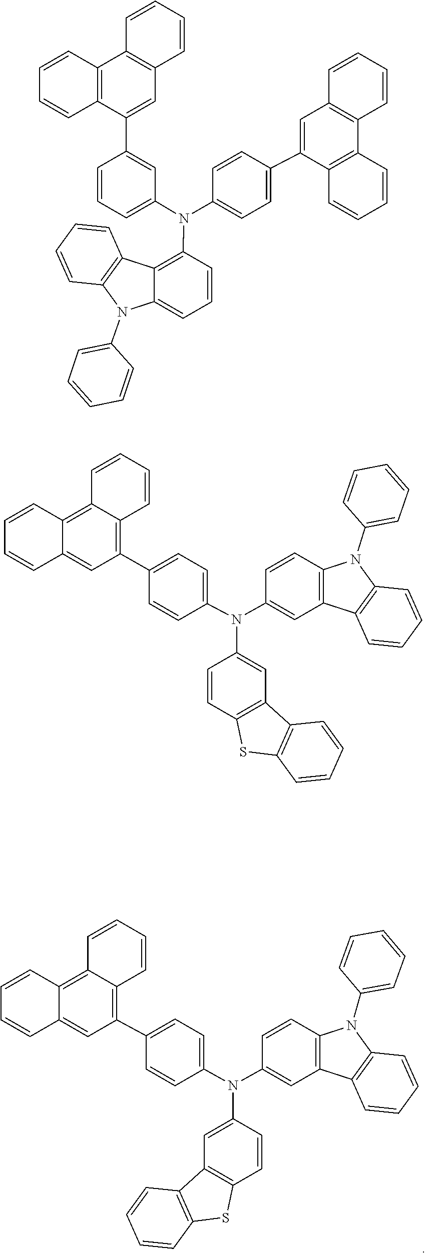 [SMILES: c1ccc(-n2c3ccccc3c3c(N(c4ccc(-c5cc6ccccc6c6ccccc56)cc4)c4cccc(-c5cc6ccccc6c6ccccc56)c4)cccc32)cc1 ; c1ccc(-n2c3ccccc3c3cc(N(c4ccc(-c5cc6ccccc6c6ccccc56)cc4)c4ccc5sc6ccccc6c5c4)ccc32)cc1 ; c1ccc(-n2c3ccccc3c3cc(N(c4ccc(-c5cc6ccccc6c6ccccc56)cc4)c4ccc5sc6ccccc6c5c4)ccc32)cc1 ; "."]
